(19) United States
(12) Patent Application Publication
(10) Pub. No.: US 2025/0171465 A1
(43) Pub. Date: May 29, 2025

(54) AKT INHIBITORS

(71) Applicant: Stablix, Inc., Boston, MA (US)

(72) Inventors: Eddine Saiah, Boston, MA (US); Tyler Mann, Boston, MA (US); Jorden Kass, Arlington, MA (US); Louise Clare Kirman, Swampscott, MA (US); Mark George Saulnier, Higganum, CT (US); Lisa A. De Meese, Silver Spring, MD (US); Richard James Steel, Foster City, CA (US); Kap-Sun Yeung, Boston, MA (US)

(21) Appl. No.: 18/752,702

(22) Filed: Jun. 24, 2024

Related U.S. Application Data
(60) Provisional application No. 63/510,039, filed on Jun. 23, 2023.

(51) Int. Cl. C07D 519/00; A61K 31/517; A61K 31/519; A61K 31/5377; A61K 31/55; A61P 35/00; C07D 401/14; C07D 403/04; C07D 413/14; C07D 487/04; C07D 493/08

(52) U.S. Cl. CPC C07D 519/00; A61K 31/517; A61K 31/519; A61K 31/5377; A61K 31/55; A61P 35/00; C07D 401/14; C07D 403/04; C07D 413/14; C07D 487/04; C07D 493/08

(57) ABSTRACT

The present invention provides compounds, compositions thereof, and methods of using the same.

### CROSS-REFERENCE TO RELATED APPLICATIONS

This application claims the benefit of priority to U.S. Provisional Appl. No. 63/510,039, filed Jun. 23, 2023, the contents of which is herein incorporated by reference.

### TECHNICAL FIELD OF THE INVENTION

The present invention relates to compounds and methods for inhibiting Protein Kinase B (“Akt”) protein via deubiquitination. The invention also provides pharmaceutically acceptable compositions comprising compounds of the present invention and methods of using said compositions in the treatment of various disorders.

### BACKGROUND OF THE INVENTION

The ubiquitination of proteins is a dynamic multifaceted post-translational modification that allows the body to mark proteins for degradation, activation, sub-cellular localization, and translocation. The best-characterized function of ubiquitin is as a marker for protein degradation. However, protein deubiquitination can result in outcomes other than preventing degradation, stabilization and enhancement of protein levels. The specific lysine being ubiquitinated influences whether the ubiquitinated protein is targeted for proteasomal degradation, activation or trafficking. For example, the most common linkage, on K48, is closely associated with degradation by the proteasome. K63-linked ubiquitination is associated with pathway activation, protein trafficking. DNA repair, and inflammation. Monoubiquitination also is not typically associated with degradation, and in some cases may regulate protein function by impeding or facilitating protein-protein association.

A deubiquitinase protein (“DUB”) is a protein that partially or fully removes ubiquitin molecules from proteins. Deubiquitinases (DUBs) can be classified into six main classes: ubiquitin-specific proteases (USPs), ubiquitin C-terminal hydrolases (UCHs), Machado-Joseph disease protein domain proteases (MJDs), ovarian tumor proteases (OTUs), JABI/MPN/MOV34 metalloenzymes (JAMMs), and motif interacting with ubiquitin-containing novel DUB family (MINDY) DUBs (Mevissen et. al., “Mechanisms of Deubiquitinase Specificity and Regulation” Annu. Rev. Biochem. 2017 (86) 159).

Bifunctional compounds composed of a target protein-binding ligand and a DUB, induced deubiquitination of selected proteins via their recruitment to the DUB. E3 ubiquitin ligase and subsequent ubiquitination. Bifunctional molecules for targeted protein deubiquitination are described in WO2021/146386A1. WO2020/169650 and Henning, Nathaniel J et al. “Deubiquitinase-targeting chimeras for targeted protein stabilization.” Nature chemical biology vol. 18,4 (2022): 412-421. doi: 10.1038/s41589-022-00971-2.

Akt (also known as Protein Kinase B (“PKB”)) is a serine/threonine protein kinase, which is a key player in growth factor and cytokine-mediated signaling cascade (Datta et al., 1999; Brazil et al., 2002; Cantley, 2002: Gonzalez and McGraw, 2009; Yang et al., 2010a). Given that Akt regulates numerous biological functions, such as cell growth, survival, cell migration, and metabolism, by phosphorylating several downstream effectors, the activity of Akt must be strictly controlled. Aberrant Akt activation is observed in various human cancers, and importantly, Akt1, Akt2, and Akt3 isoforms are found to be overexpressed in human cancers (Staal, 1987: Cheng et al., 1992, 1996; Bellacosa et al., 1995; Nakatani et al., 1999; Stahl et al., 2004). Recent studies show that Akt1 mutations are observed in a subset of human cancers and are associated with Akt hyperactivation (Carpten et al., 2007: Kim et al., 2008a: Malanga et al., 2008; Mohamedali et al., 2008; Askham et al., 2010; Shoji et al., 2009; Zilberman et al., 2009). The role of Akt in cancer development has been supported by numerous animal tumor models. For example, Pten+/− mice with aberrant Akt activation develop multiple tumors, which can be inhibited by Akt1 deficiency (Di Cristofano et al., 2001: Chen et al., 2006). In addition, the prostate-specific expression of constitutively active Akt1 in mice leads to prostate intraepithelial neoplasia (Majumder et al., 2003, 2004). These results highlight the critical role of the Akt pathway in cancer development. Accordingly, there remains a need to find compounds that are Akt inhibitors useful as therapeutic agents.

### SUMMARY OF THE INVENTION

The present application relates to novel bifunctional compounds, which function to recruit Akt protein to DUBs for deubiquitination, and methods of preparation and uses thereof. In particular, the present disclosure provides bifunctional compounds, which find utility as inducers of targeted deubiquitination of Akt resulting in inhibition.

It has now been found that compounds of this invention, and pharmaceutically acceptable compositions thereof, are effective as inhibitors of Akt protein. Such compounds have the general formula I:

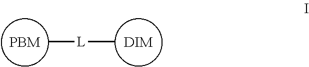

or a pharmaceutically acceptable salt thereof, wherein each variable is as defined and described herein.

Compounds of the present invention, and pharmaceutically acceptable compositions thereof, are useful for treating a variety of diseases, disorders or conditions, associated with regulation of Akt protein. Such diseases, disorders, or conditions include those described herein.

### DETAILED DESCRIPTION OF SOME EMBODIMENTS

### 1. General Description of Some Embodiments of the Invention:

Compounds of the present invention, and compositions thereof, are useful as inhibitors of Akt protein. In some embodiments, a provided compound inhibits Akt protein.

In some embodiments, the present invention provides a compound of formula I:

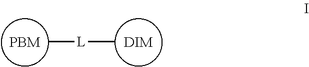

or a pharmaceutically acceptable salt thereof, wherein:
PBM is a protein binding moiety capable of binding Akt;
L is a bivalent moiety that connects PBM to DIM; and
DIM is a deubiquitination inducing moiety, such as a deubiquitinase binding moiety (DBM).

### 2. Compounds and Definitions:

Compounds and compositions described herein are generally useful for the inhibition of kinase activity of one or more enzymes.

The terms “Akt protein” and “Akt” refer to all members of the Akt protein kinase family, including, but not limited to Akt1 (PKBa), Akt2 (PKBB), and Akt3 (PKBY).

Compounds of the present invention include those described generally herein, and are further illustrated by the classes, subclasses, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75th Ed. Additionally, general principles of organic chemistry are described in “Organic Chemistry”, Thomas Sorrell, University Science Books, Sausalito: 1999, and “March's Advanced Organic Chemistry”, 5th Ed., Ed.: Smith, M. B, and March, J., John Wiley & Sons, New York: 2001, the entire contents of which are hereby incorporated by reference.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic, bicyclic, bridged bicyclic, or spirocyclic hydrocarbon that is completely saturated or that contains one or more units of unsaturation, but which is not aromatic (also referred to herein as “carbocycle,” “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle” or “cycloalkyl”) refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

As used herein, the term “bridged bicyclic” refers to any bicyclic ring system, i.e. carbocyclic or heterocyclic, saturated or partially unsaturated, having at least one bridge. As defined by IUPAC, a “bridge” is an unbranched chain of atoms or an atom or a valence bond connecting two bridgeheads, where a “bridgehead” is any skeletal atom of the ring system which is bonded to three or more skeletal atoms (excluding hydrogen). In some embodiments, a bridged bicyclic group has 7-12 ring members and 0-4 heteroatoms independently selected from nitrogen, oxygen, or sulfur. Such bridged bicyclic groups are well known in the art and include those groups set forth below where each group is attached to the rest of the molecule at any substitutable carbon or nitrogen atom. Unless otherwise specified, a bridged bicyclic group is optionally substituted with one or more substituents as set forth for aliphatic groups. Additionally or alternatively, any substitutable nitrogen of a bridged bicyclic group is optionally substituted. Exemplary bridged bicyclics include:

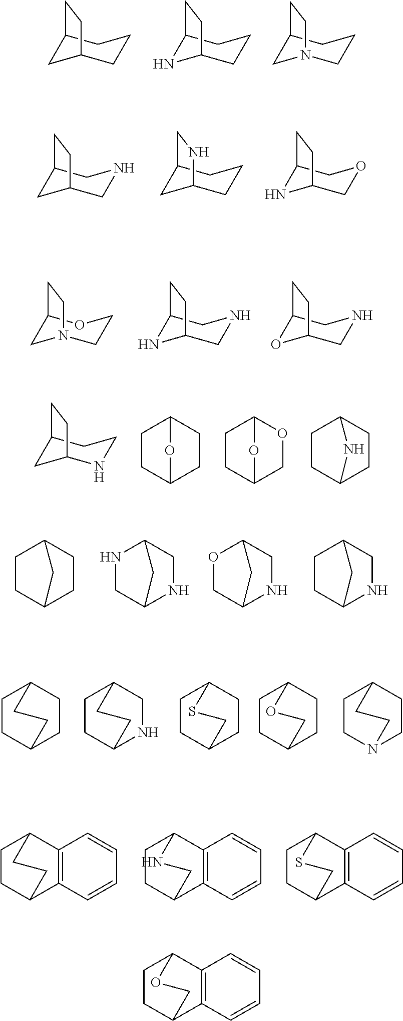

The term “lower alkyl” refers to a C₁₋₄ straight or branched alkyl group. Exemplary lower alkyl groups are methyl, ethyl, propyl, isopropyl, butyl, isobutyl, and tert-butyl.

The term “lower haloalkyl” refers to a C₁₋₄ straight or branched alkyl group that is substituted with one or more halogen atoms.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

The term “unsaturated,” as used herein, means that a moiety has one or more units of unsaturation.

As used herein, the term “bivalent C₁₋₈ (or C₁₋₆) saturated or unsaturated, straight or branched, hydrocarbon chain”, refers to bivalent alkylene, alkenylene, and alkynylene chains that are straight or branched as defined herein.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

As used herein, a term containing the suffix “-ylenyl” generally refers to a bivalent group. For example, as used herein, the term “cyclopropylenyl” refers to a bivalent cyclopropyl group, for example, of the following structure:

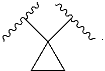 [SMILES: *C1(*)CC1]

In some embodiments, the suffix “-ylenyl” may refer to a moiety that can be a monovalent structure. For example, if ring X as depicted below:

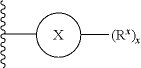

is described or defined as phenylenyl, one of skill in the art would understand that if x is 0 then ring X is monovalent, i.e., the only substituents on the ring are hydrogen.

The term “halogen” means F, Cl, Br, or I.

The term “aryl” used alone or as part of a larger moiety as in “aralkyl,” “aralkoxy,” or “aryloxyalkyl,” refers to monocyclic or bicyclic ring systems having a total of five to fourteen ring members, wherein at least one ring in the system is aromatic and wherein each ring in the system contains 3 to 7 ring members. The term “aryl” may be used interchangeably with the term “aryl ring.” In some embodiments of the present invention, “aryl” refers to an aromatic ring system which includes, but not limited to, phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-,” used alone or as part of a larger moiety, e.g., “heteroaralkyl,” or “heteroaralkoxy,” refer to groups having 5 to 10 ring atoms, preferably 5, 6, or 9 ring atoms; having 6, 10, or 14π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3 (4H)-one. A heteroaryl group may be mono- or bicyclic. A heteroaryl ring may include one or more oxo (═O) or thioxo (═S) substituent. The term “heteroaryl” may be used interchangeably with the terms “heteroaryl ring.” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle,” “heterocyclyl.” “heterocyclic radical,” and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H pyrrolyl), NH (as in pyrrolidinyl), or ⁺NR (as in N substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle.” “heterocyclyl.” “heterocyclyl ring.” “heterocyclic group.” “heterocyclic moiety.” and “heterocyclic radical,” are used interchangeably herein, and also include groups in which a heterocyclyl ring is fused to one or more aryl, heteroaryl, or cycloaliphatic rings, such as indolinyl, 3H indolyl, chromanyl, phenanthridinyl, or tetrahydroquinolinyl. A heterocyclyl group may be monocyclic, bicyclic, bridged bicyclic, or spirocyclic. A heterocyclic ring may include one or more oxo (═O) or thioxo (═S) substituent. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, compounds of the invention may contain “optionally substituted” moieties. In general, the term “substituted” means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in some embodiments, their recovery, purification, and use for one or more of the purposes disclosed herein.

Suitable monovalent substituents on a substitutable carbon atom of an “optionally substituted” group are independently halogen; —(CH₂)₀₋₄R^{◯}; —(CH₂)₀₋₄OR^{◯}; —O(CH₂)₀₋₄R^{◯}, —O—(CH₂)₀₋₄C(O)OR^{◯}; —(CH₂)₀₋₄CH(OR^{◯})₂; —(CH₂)₀₋₄SR^{◯}; —(CH₂)₀₋₄Ph, which may be substituted with R^{◯}; —(CH₂)₀₋₄O(CH₂)₀₋₁Ph which may be substituted with R^{◯}; —CH═CHPh, which may be substituted with R^{◯}; —(CH₂)₀₋₄O(CH₂)₀₋₁-pyridyl which may be substituted with R^{◯}; —NO₂; —CN; —N₃; —(CH₂)₀₋₄N(R^{◯})₂; —(CH₂)₀₋₄N(R^{◯})C(O)R^{◯}; —N(R^{◯})C(S)R^{◯}; —(CH₂)₀₋₄N(R^{◯})C(O)NR^{◯}₂; —N(R^{◯})C(S)NR^{◯}₂; —(CH₂)₀₋₄N(R^{◯})C(O)OR^{◯}; —N(R^{◯})N(R^{◯})C(O)R^{◯}; —N(R^{◯})N(R^{◯})C(O)NR^{◯}₂; —N(R^{◯})N(R^{◯})C(O)OR^{◯}; —(CH₂)₀₋₄C(O)R^{◯}; —C(S)R^{◯}; —(CH₂)₀₋₄C(O)OR^{◯}; —(CH₂)₀₋₄C(O)SR^{◯}; —(CH₂)₀₋₄C(O)OSiR^{◯}₃; —(CH₂)₀₋₄OC(O)R^{◯}; —OC(O)(CH₂)₀₋₄SR^{◯}; —(CH₂)₀₋₄SC(O)R^{◯}; —(CH₂)₀₋₄C(O)NR^{◯}₂; —C(S)NR^{◯}₂; —C(S)SR^{◯}; —SC(S)SR^{◯}, —(CH₂)₀₋₄OC(O)NR^{◯}₂; —C(O)N(OR^{◯})R^{◯}; —C(O)C(O)R^{◯}; —C(O)CH₂C(O)R^{◯}; —C(NOR^{◯})R^{◯}; —(CH₂)₀₋₄SSR^{◯}; —(CH₂)₀₋₄S(O)₂R^{◯}; —(CH₂)₀₋₄S(O)₂OR^{◯}; —(CH₂)₀₋₄OS(O)₂R^{◯}; —S(O)₂NR^{◯}₂; —(CH₂)₀₋₄S(O)R^{◯}; —N(R^{◯})S(O)₂NR^{◯}₂; —N(R^{◯})S(O)₂R^{◯}; —N(OR^{◯})R^{◯}; —C(NH)NR^{◯}₂; —(CH₂)₀₋₄P(O)₂R^{◯}; —(CH₂)₀₋₄P(O)R^{◯}₂; —(CH₂)₀₋₄P(O)(OR^{◯})₂; —(CH₂)₀₋₄OP(O)R^{◯}₂; —(CH₂)₀₋₄OP(O)(OR^{◯})₂; SiR^{◯}₃; —(C₁₋₄ straight or branched alkylene) O—N(R^{◯})₂; or —(C₁₋₄ straight or branched alkylene)C(O)O—N(R^{◯})₂, wherein each R^{◯} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{◯}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{◯} (or the ring formed by taking two independent occurrences of R^{◯} together with their intervening atoms), are independently halogen, —(CH₂)₀₋₂R^{●}, -(haloR^{●}), —(CH₂)₀₋₂OH, —(CH₂)₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{◯} include ═O and ═S.

Suitable divalent substituents on a saturated carbon atom of an “optionally substituted” group include the following: ═O, ═S, ═NNR*₂, =NNHC(O)R*, =NNHC(O)OR*, =NNHS(O)₂R*, =NR*, =NOR*, —O(C(R*₂)₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate. In some embodiments, the provided compounds are purified in salt form for convenience and/or ease of purification, e.g., using an acidic or basic mobile phase during chromatography. Salts forms of the provided compounds formed during chromotagraphic purification are contemplated herein and are readily apparent to those having skill in the art.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a 13C- or 14C-enriched carbon are within the scope of this invention. Such compounds are useful, for example, as analytical tools, as probes in biological assays, or as therapeutic agents in accordance with the present invention

As used herein, the term “provided compound” refers to any genus, subgenus, and/or species set forth herein.

As used herein, the term “inhibitor” is defined as a compound that binds to and/or inhibits Akt with measurable affinity. In some embodiments, an inhibitor has an IC₅₀ and/or binding constant of less than about 50 uM, less than about 1 uM, less than about 500 nM, less than about 100 nM, less than about 10 nM, or less than about 1 nM.

The terms “measurable affinity” and “measurably inhibit,” as used herein, means a measurable change in Akt activity between a sample comprising a compound of the present invention, or composition thereof, and Akt, and an equivalent sample comprising Akt, in the absence of said compound, or composition thereof.

As used herein, the term “independently” means independently for each occurrence.

### 3. Description of Exemplary Embodiments:

As described above, in some embodiments, the present invention provides a compound of formula I:

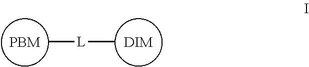

or a pharmaceutically acceptable salt thereof, wherein:
PBM is a protein binding moiety capable of binding Akt;
L is a bivalent moiety that connects PBM to DIM; and
DIM is a deubiquitination inducing moiety, such as a deubiquitinase binding moiety (DBM).

### Protein Binding Moiety (PBM)

As defined herein and described above, PBM is a protein binding moiety capable of binding Akt. In some embodiments, PBM binds to Akt protein which then undergoes deubiquitination thereby deactivating the Akt protein.

As defined herein and described below, wherein a formula is depicted using square brackets, e.g.,

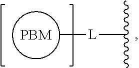

L is attached to a modifiable carbon, oxygen, nitrogen or sulfur atom within PBM including substitution or replacement of a defined group in PBM.

In some embodiments, the present invention provides a compound of formula I, wherein PBM is an Akt binding moiety thereby forming a compound of formula I-a:

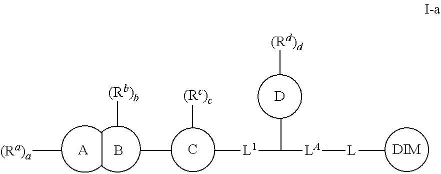

or a pharmaceutically acceptable salt thereof, wherein L and DIM are as defined above and described herein, and wherein:
Ring A and Ring B are independently fused rings selected from benzo, a 4-7 membered saturated or partially unsaturated carbocyclyl, a 4-7 membered saturated or partially unsaturated heterocyclyl with 1-3 heteroatoms independently selected from nitrogen, oxygen, and sulfur, and a 5-6 membered heteroaryl with 1-4 heteroatoms independently selected from nitrogen, oxygen and sulfur;
Ring C is a ring selected from phenylenyl, a 4-7 membered saturated or partially unsaturated carbocyclylenyl, an 8-10 membered saturated or partially unsaturated bridged bicyclic carbocyclylenyl, a 4-7 membered saturated or partially unsaturated heterocyclylenyl with 1-3 heteroatoms independently selected from nitrogen, oxygen, and sulfur, an 8-10 membered saturated or partially unsaturated bridged bicyclic heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
Ring D is a ring selected from phenyl, a 4-7 membered saturated or partially unsaturated carbocyclyl, a 4-7 membered saturated or partially unsaturated heterocyclyl with 1-3 heteroatoms independently selected from nitrogen, oxygen, and sulfur, and a 5-6 membered heteroaryl with 1-4 heteroatoms independently selected from nitrogen, oxygen and sulfur;
L¹ and L^{A} are independently a covalent bond or a bivalent, saturated or partially unsaturated, straight or branched C₁₋₅ hydrocarbon chain, wherein 0-3 methylene units of L¹ or L^{A} are independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, or —OC(O)NR—, —NRC(O)O—;
each R^{a}, R^{b}, R^{c}, and R^{d} is independently selected from hydrogen, R^{A}, halogen, —CN, —NO₂, —OR, —SR, —NR₂, —S(O)₂R, —S(O)₂NR₂, —S(O)R, —C(O)R, —C(O)OR, —C(O)NR₂, —C(O)NROR, —OC(O)R, —OC(O)NR₂, —NRC(O)OR, —NRC(O)R, —NRC(O)N(R)₂, and —NRS(O)₂R;
each R^{A} is independently an optionally substituted group selected from C₁₋₆ aliphatic, phenyl, a 3-12 membered saturated or partially unsaturated monocyclic, bicyclic, bridged bicyclic or spirocyclic carbocyclyl or heterocyclyl with 1-3 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or;
   two R groups on the same nitrogen are optionally taken together with their intervening atoms to form a 4-7 membered saturated, partially unsaturated, or heteroaryl ring having 0-3 heteroatoms, in addition to the nitrogen atom to which they are attached, independently selected from nitrogen, oxygen, and sulfur; and
a, b, c, and d are independently 0, 1, 2, 3, or 4.

In some embodiments, the present invention provides a compound of formula I, wherein PBM is an Akt binding moiety thereby forming a compound of formula I-a′:

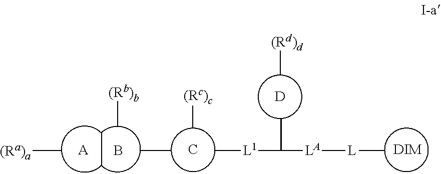

or a pharmaceutically acceptable salt thereof, wherein L and DIM are as defined above and described herein, and wherein:
Ring A and Ring B are independently fused rings selected from benzo, a 4-7 membered saturated or partially unsaturated carbocyclyl, a 4-7 membered saturated or partially unsaturated heterocyclyl with 1-3 heteroatoms independently selected from nitrogen, oxygen, and sulfur, and a 5-6 membered heteroaryl with 1-4 heteroatoms independently selected from nitrogen, oxygen and sulfur;
Ring C is a ring selected from phenylenyl, a 4-7 membered saturated or partially unsaturated carbocyclylenyl, a 8-10 membered saturated or partially unsaturated bridged bicyclic carbocyclylenyl, a 4-7 membered saturated or partially unsaturated heterocyclylenyl with 1-3 heteroatoms independently selected from nitrogen, oxygen, and sulfur, a 8-10 membered saturated or partially unsaturated bridged bicyclic heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
Ring D is a ring selected from phenyl, a 4-7 membered saturated or partially unsaturated carbocyclyl, a 4-7 membered saturated or partially unsaturated heterocyclyl with 1-3 heteroatoms independently selected from nitrogen, oxygen, and sulfur, and a 5-6 membered heteroaryl with 1-4 heteroatoms independently selected from nitrogen, oxygen and sulfur;
L¹ is a covalent bond or a bivalent, saturated or partially unsaturated, straight or branched C₁₋₅ hydrocarbon chain, wherein 0-3 methylene units of L¹ or L^{A} are independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, or —OC(O)NR—, —NRC(O)O—;
L^{A} is a covalent bond or a bivalent, saturated or partially unsaturated, straight or branched C₁₋₅ hydrocarbon chain, wherein 0-3 methylene units of L¹ or L^{A} are independently replaced by -Cy-, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, or —OC(O)NR—, —NRC(O)O—;
each -Cy- is independently an optionally substituted bivalent ring selected from phenylenyl, an 8-10 membered bicyclic arylenyl, a 4-7 membered saturated or partially unsaturated carbocyclylenyl, a 6-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 6-11 membered saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, and an 8-10 membered bicyclic heteroarylenyl having 1-5 heteroatoms independently selected from nitrogen, oxygen, and sulfur;
each R^{a}, R^{b}, R^{c}, and R^{d} is independently selected from hydrogen, R^{A}, halogen, —CN, —NO₂, —OR, —SR, —NR₂, —S(O)₂R, —S(O)₂NR₂, —S(O)R, —C(O)R, —C(O)OR, —C(O)NR₂, —C(O)NROR, —OC(O)R, —OC(O)NR₂, —NRC(O)OR, —NRC(O)R, —NRC(O)N(R)₂, and —NRS(O)₂R;
each R^{A} is independently an optionally substituted group selected from C₁₋₆ aliphatic, phenyl, a 3-12 membered saturated or partially unsaturated monocyclic, bicyclic, bridged bicyclic or spirocyclic carbocyclyl or heterocyclyl with 1-3 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or;
   two R groups on the same nitrogen are optionally taken together with their intervening atoms to form a 4-7 membered saturated, partially unsaturated, or heteroaryl ring having 0-3 heteroatoms, in addition to the nitrogen atom to which they are attached, independently selected from nitrogen, oxygen, and sulfur; and
a, b, c, and d are independently 0, 1, 2, 3, or 4.

In some embodiments, the present invention provides a compound of formula I-a, wherein PBM is an Akt binding moiety thereby forming a compound of formula I-b:

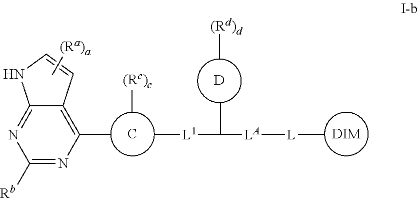

or a pharmaceutically acceptable salt thereof, wherein L and DIM are as defined above and described herein, and wherein;
Ring C is a ring selected from a 4-7 membered saturated or partially unsaturated carbocyclylenyl, a 3-12 membered saturated or partially unsaturated bridged bicyclic carbocyclylenyl, a 4-7 membered saturated or partially unsaturated heterocyclylenyl with 1-3 heteroatoms independently selected from nitrogen, oxygen, and sulfur, a 3-12 membered saturated or partially unsaturated bridged bicyclic heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
Ring D is a ring selected from phenyl and a 5-6 membered heteroaryl with 1-4 heteroatoms independently selected from nitrogen, oxygen and sulfur;
L¹ is a covalent bond, —CR₂—, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, or —OC(O)NR—, —NRC(O)O—;
L^{A} is a covalent bond or a bivalent, saturated or partially unsaturated, straight or branched C₁₋₅ hydrocarbon chain, wherein 0-3 methylene units of L^{A} are independently replaced by —O—, —NR—, —S—, —OC(O)—, —C(O)O—, —C(O)—, —S(O)—, —S(O)₂—, —NRS(O)₂—, —S(O)—NR—, —NRC(O)—, —C(O)NR—, or —OC(O)NR—, —NRC(O)O—;
each R^{a}, R^{b}, R^{c}, and R^{d} is independently selected from hydrogen, R^{A}, halogen, —CN, —NO₂, —OR, —SR, —NR₃, —S(O)₂R, —S(O)₂NR₂, —S(O)R, —C(O)R, —C(O)OR, —C(O)NR₂, —C(O)NROR, —OC(O)R, —OC(O)NR₂, —NRC(O)OR, —NRC(O)R, —NRC(O)N(R)₂, and —NRS(O)₂R;
each R^{A} is independently an optionally substituted group selected from C₁₋₆ aliphatic, phenyl, a 3-12 membered saturated or partially unsaturated monocyclic, bicyclic, bridged bicyclic or spirocyclic carbocyclyl or heterocyclyl with 1-3 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or;
   two R groups on the same nitrogen are optionally taken together with their intervening atoms to form a 4-7 membered saturated, partially unsaturated, or heteroaryl ring having 0-3 heteroatoms, in addition to the nitrogen atom to which they are attached, independently selected from nitrogen, oxygen, and sulfur; and
a, b, c, and d are independently 0, 1, 2, 3, or 4.

In some embodiments, the present invention provides a compound of formula I-a′, wherein PBM is an Akt binding moiety thereby forming a compound of formula I-b′:

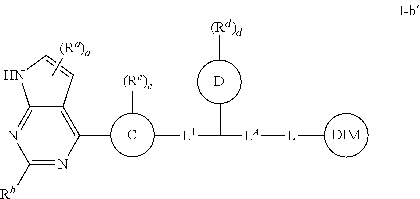

or a pharmaceutically acceptable salt thereof, wherein L and DIM are as defined above and described herein, and wherein:
Ring C is a ring selected from a 4-7 membered saturated or partially unsaturated carbocyclylenyl, a 3-12 membered saturated or partially unsaturated bridged bicyclic carbocyclylenyl, a 4-7 membered saturated or partially unsaturated heterocyclylenyl with 1-3 heteroatoms independently selected from nitrogen, oxygen, and sulfur, a 3-12 membered saturated or partially unsaturated bridged bicyclic heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
Ring D is a ring selected from phenyl and a 5-6 membered heteroaryl with 1-4 heteroatoms independently selected from nitrogen, oxygen and sulfur;
L¹ is a covalent bond, —CR₂—, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, or —OC(O)NR—, —NRC(O)O—;
L^{A} is a covalent bond or a bivalent, saturated or partially unsaturated, straight or branched C₁₋₅ hydrocarbon chain, wherein 0-3 methylene units of L^{A} are independently replaced by -Cy-, —O—, —NR—, —S—, —OC(O)—, —C(O)O—, —C(O)—, —S(O)—, —S(O)₂—, —NRS(O)₂—, —S(O)—NR—, —NRC(O)—, —C(O)NR—, or —OC(O)NR—, —NRC(O)O—;
each -Cy- is independently an optionally substituted bivalent ring selected from phenylenyl, an 8-10 membered bicyclic arylenyl, a 4-7 membered saturated or partially unsaturated carbocyclylenyl, a 6-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 6-11 membered saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, and an 8-10 membered bicyclic heteroarylenyl having 1-5 heteroatoms independently selected from nitrogen, oxygen, and sulfur;
each R^{a}, R^{b}, R^{c}, and R^{d} is independently selected from hydrogen, R^{A}, halogen, —CN, —NO₂, —OR, —SR, —NR₂, —S(O)₂R, —S(O)₂NR₂, —S(O)R, —C(O)R, —C(O)OR, —C(O)NR₂, —C(O)NROR, —OC(O)R, —OC(O)NR₂, —NRC(O)OR, —NRC(O)R, —NRC(O)N(R)₂, and —NRS(O)₂R;
each R^{A} is independently an optionally substituted group selected from C₁₋₆ aliphatic, phenyl, a 3-12 membered saturated or partially unsaturated monocyclic, bicyclic, bridged bicyclic or spirocyclic carbocyclyl or heterocyclyl with 1-3 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or;
   two R groups on the same nitrogen are optionally taken together with their intervening atoms to form a 4-7 membered saturated, partially unsaturated, or heteroaryl ring having 0-3 heteroatoms, in addition to the nitrogen atom to which they are attached, independently selected from nitrogen, oxygen, and sulfur; and
a, b, c, and d are independently 0, 1, 2, 3, or 4.

In some embodiments, the present invention provides a compound of formula I-b or I-b′, wherein PBM is an Akt binding moiety thereby forming a compound of formula I-b-1:

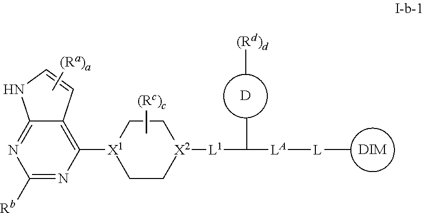

or a pharmaceutically acceptable salt thereof, wherein each of ring D, L¹, L^{A}, R^{a}, R^{b}, R^{c}, R^{d}, a, b, c, d, L, and DIM is as defined above and described in embodiments herein, both singly and in combination, wherein X¹ and X² are independently nitrogen or carbon.

In some embodiments, the present invention provides a compound of formula I-a, wherein PBM is an Akt binding moiety thereby forming a compound of formula I-c:

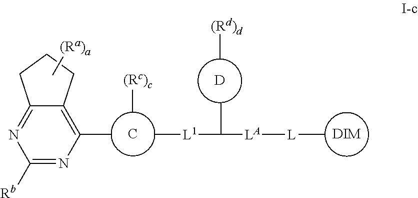

or a pharmaceutically acceptable salt thereof, wherein L and DIM are as defined above and described herein, and wherein:
Ring C is a ring selected from a 4-7 membered saturated or partially unsaturated carbocyclylenyl, a 3-12 membered saturated or partially unsaturated bridged bicyclic carbocyclylenyl, a 4-7 membered saturated or partially unsaturated heterocyclylenyl with 1-3 heteroatoms independently selected from nitrogen, oxygen, and sulfur, a 3-12 membered saturated or partially unsaturated bridged bicyclic heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
Ring D is a ring selected from phenyl, a 4-7 membered saturated or partially unsaturated carbocyclyl, a 4-7 membered saturated or partially unsaturated heterocyclyl with 1-3 heteroatoms independently selected from nitrogen, oxygen, and sulfur, and a 5-6 membered heteroaryl with 1-4 heteroatoms independently selected from nitrogen, oxygen and sulfur;
L¹ is a covalent bond, —CR₂—, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, or —OC(O)NR—, —NRC(O)O—;
L^{A} is a covalent bond or a bivalent, saturated or partially unsaturated, straight or branched C₁₋₅ hydrocarbon chain, wherein 0-3 methylene units of L^{A} are independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, or —OC(O)NR—, —NRC(O)O—;
each R^{a}, R^{b}, R^{c}, and R^{d} is independently selected from hydrogen, R^{A}, halogen, —CN, —NO₂, —OR, —SR, —NR₂, —S(O)₂R, —S(O)₂NR₂, —S(O)R, —C(O)R, —C(O)OR, —C(O)NR₂, —C(O)NROR, —OC(O)R, —OC(O)NR₂, —NRC(O)OR, —NRC(O)R, —NRC(O)N(R)₂, and —NRS(O)₂R;
each R^{A} is independently an optionally substituted group selected from C₁₋₆ aliphatic, phenyl, a 3-12 membered saturated or partially unsaturated monocyclic, bicyclic, bridged bicyclic or spirocyclic carbocyclyl or heterocyclyl with 1-3 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or;
   two R groups on the same nitrogen are optionally taken together with their intervening atoms to form a 4-7 membered saturated, partially unsaturated, or heteroaryl ring having 0-3 heteroatoms, in addition to the nitrogen atom to which they are attached, independently selected from nitrogen, oxygen, and sulfur; and
a, b, c, and d are independently 0, 1, 2, 3, or 4.

In some embodiments, the present invention provides a compound of formula I-a′, wherein PBM is an Akt binding moiety thereby forming a compound of formula I-c′:

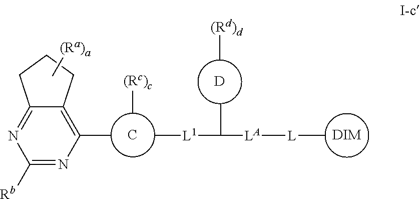

or a pharmaceutically acceptable salt thereof, wherein L and DIM are as defined above and described herein, and wherein:
Ring C is a ring selected from a 4-7 membered saturated or partially unsaturated carbocyclylenyl, a 3-12 membered saturated or partially unsaturated bridged bicyclic carbocyclylenyl, a 4-7 membered saturated or partially unsaturated heterocyclylenyl with 1-3 heteroatoms independently selected from nitrogen, oxygen, and sulfur, a 3-12 membered saturated or partially unsaturated bridged bicyclic heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
Ring D is a ring selected from phenyl, a 4-7 membered saturated or partially unsaturated carbocyclyl, a 4-7 membered saturated or partially unsaturated heterocyclyl with 1-3 heteroatoms independently selected from nitrogen, oxygen, and sulfur, and a 5-6 membered heteroarylenyl with 1-4 heteroatoms independently selected from nitrogen, oxygen and sulfur;
L¹ is a covalent bond, —CR₂—, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, or —OC(O)NR—, —NRC(O)O—;
L^{A} is a covalent bond or a bivalent, saturated or partially unsaturated, straight or branched C₁₋₅ hydrocarbon chain, wherein 0-3 methylene units of L^{A} are independently replaced by -Cy-, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, or —OC(O)NR—, —NRC(O)O—;
each -Cy- is independently an optionally substituted bivalent ring selected from phenylenyl, an 8-10 membered bicyclic arylenyl, a 4-7 membered saturated or partially unsaturated carbocyclylenyl, a 6-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 6-11 membered saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, and an 8-10 membered bicyclic heteroarylenyl having 1-5 heteroatoms independently selected from nitrogen, oxygen, and sulfur;
each R^{a}, R^{b}, R^{c}, and R^{d} is independently selected from hydrogen, R^{A}, halogen, —CN, —NO₂, —OR, —SR, —NR₂, —S(O)₂R, —S(O)₂NR₂, —S(O)R, —C(O)R, —C(O)OR, —C(O)NR₂, —C(O)NROR, —OC(O)R. —OC(O)NR₂, —NRC(O)OR, —NRC(O)R, —NRC(O)N(R)₂, and —NRS(O)₂R;
each R^{A} is independently an optionally substituted group selected from C₁₋₆ aliphatic, phenyl, a 3-12 membered saturated or partially unsaturated monocyclic, bicyclic, bridged bicyclic or spirocyclic carbocyclyl or heterocyclyl with 1-3 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or;
   two R groups on the same nitrogen are optionally taken together with their intervening atoms to form a 4-7 membered saturated, partially unsaturated, or heteroaryl ring having 0-3 heteroatoms, in addition to the nitrogen atom to which they are attached, independently selected from nitrogen, oxygen, and sulfur; and
a, b, c, and d are independently 0, 1, 2, 3, or 4.

In some embodiments, the present invention provides a compound of formula I-c or I-c′, wherein PBM is an Akt binding moiety thereby forming a compound of formula I-c-1:

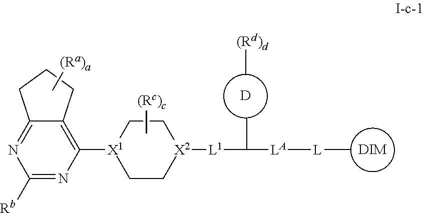

or a pharmaceutically acceptable salt thereof, wherein each of ring D, L¹, L^{A}, R^{a}, R^{b}, R^{c}, R^{d}, a, b, c, d, L, and DIM is as defined above and described in embodiments herein, both singly and in combination, wherein X¹ and X² are independently nitrogen or carbon.

In some embodiments, a provided compound or pharmaceutically acceptable salt thereof, is selected from those wherein PBM is A-443654, A-674563, Afuresertib (GSK2110183), Akt Kinase Inhibitor (CAS No.: 842148-40-7), (E)-Akt inhibitor-IV ((E)-AKTIV), Akt Inhibitor XIX (3CAI), Akt2-IN-1, Akt2-IN-2, Akt1-IN-1, Akti-1/2 (Akt Inhibitor VIII), Akt-IN-1, Akt-IN-10, Akt-IN-12, Akt-IN-13, Akt-IN-2, Akt-IN-3, Akt-IN-5, Akt-IN-6, Akt-IN-7, Akt-IN-8, Akt-IN-9, ALM301, Alobresib (GS-5829), API-1, AT13148, AT7867, BAY 1125976, Borussertib, Capivasertib (AZD5363), CCT128930, Cinobufagin (Cinobufagine), Daphnoretin (Dephnoretin; Thymelol), DB07107, Deguelin ((−)-Deguelin; (−)-cis-Deguelin), Esculetin, FPA-124, GSK2110183 analog 1, GSK690693, Guggulsterone, Hematein, Honokiol (NSC 293100), Hu7691, Ipatasertib (GDC0068), Isoginkgetin, KP372-1, Loureirin A, M2698 (MSC2363318A), 24-Methylenecycloartanyl ferulate, Methyl-Hesperidin, Miltefosine (Hexadecylphosphocholine), Miransertib (ARQ092), MK-2206, ML-9 HCl, MMP-9-IN-3, MMP-9-IN-5, NTQ1062, oridonin (NSC-250682), Oroxin B (Hypocretin-2), Pachymic acid (3-O-Acetyltumulosic acid), Perifosine (KRX-0401), PF-04691502, PF-AKT400, PHT-427 (CS-0223), PI3K/Akt/mTOR-IN-3, Pifusertib (TAS-117), PP2A Cancerous-IN-1, Praeruptorin A, Resibufogenin (Bufogenin; Recibufogenin), RPI-1, SC66, Scutellarin (Breviscapine; Breviscapin; Scutellarein-7-glucuronide), SM-020, SPP-86, SR13668, STL1 (ZINC2429155), swertiamarin, TAS0612, TASP0415914, TIC10 (ONC201), TIC10 Analogue, Triciribine, Trigonelline (Trigenolline), Uprosertib (GSK2141795), Urolithin B, Usnic acid (Usniacin), or Vevorisertib (ARQ 751).

In some embodiments, PBM is capivasertib (AZD5363).

In some embodiments, L is attached to a modifiable carbon, oxygen, nitrogen or sulfur atom within PBM including substitution or replacement of a defined group in PBM. For example, in some embodiments, PBM comprises, e.g., a hydroxyl group that can be modified to create a leaving group that is subsequently replaced by a nucleophilic group of L such that PBM and L are thereby attached. One of skill in the art will appreciate that various methods are available in the art to facilitate such modifications.

As defined above and described herein, Ring A and Ring B are independently fused rings selected from benzo, a 4-7 membered saturated or partially unsaturated carbocyclyl, a 4-7 membered saturated or partially unsaturated heterocyclyl with 1-3 heteroatoms independently selected from nitrogen, oxygen, and sulfur, and a 5-6 membered heteroaryl with 1-4 heteroatoms independently selected from nitrogen, oxygen and sulfur.

In some embodiments, Ring A is a fused benzo ring. In some embodiments, Ring A is a 4-7 membered saturated or partially unsaturated carbocyclyl. In some embodiments, Ring A is a 4-7 membered saturated or partially unsaturated heterocyclyl with 1-3 heteroatoms independently selected from nitrogen, oxygen, and sulfur. In some embodiments, Ring A is a 5-6 membered heteroaryl with 1-4 heteroatoms independently selected from nitrogen, oxygen and sulfur.

In some embodiments, ring A is selected from those depicted in the compounds of Table 1 below.

In some embodiments, Ring B is a fused benzo ring. In some embodiments, Ring B is a 4-7 membered saturated or partially unsaturated carbocyclyl. In some embodiments, Ring B is a 4-7 membered saturated or partially unsaturated heterocyclyl with 1-3 heteroatoms independently selected from nitrogen, oxygen, and sulfur. In some embodiments, Ring B is a 5-6 membered heteroaryl with 1-4 heteroatoms independently selected from nitrogen, oxygen and sulfur.

In some embodiments, ring B is selected from those depicted in the compounds of Table 1 below.

As defined above and described herein, Ring C is a ring selected from phenylenyl, a 4-7 membered saturated or partially unsaturated carbocyclylenyl, a 8-10 membered saturated or partially unsaturated bridged bicyclic carbocyclylenyl, a 4-7 membered saturated or partially unsaturated heterocyclylenyl with 1-3 heteroatoms independently selected from nitrogen, oxygen, and sulfur, a 8-10 membered saturated or partially unsaturated bridged bicyclic heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur.

In some embodiments, ring C is a phenylenyl. In some embodiments, ring C is a 4-7 membered saturated or partially unsaturated carbocyclylenyl. In some embodiments, ring C is a 8-10 membered saturated or partially unsaturated bridged bicyclic carbocyclylenyl. In some embodiments, ring C is a 4-7 membered saturated or partially unsaturated heterocyclylenyl with 1-3 heteroatoms independently selected from nitrogen, oxygen, and sulfur. In some embodiments, ring C is a 8-10 membered saturated or partially unsaturated bridged bicyclic heterocyclylenyl with 1-3 heteroatoms independently selected from nitrogen, oxygen, and sulfur. In some embodiments, ring C is a 5-6 membered heteroarylenyl with 1-4 heteroatoms independently selected from nitrogen, oxygen and sulfur.

In some embodiments, ring C is

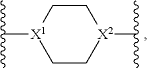

wherein X¹ and X² are independently carbon or nitrogen. In some embodiments, ring C is

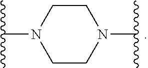 [SMILES: *N1CCN(*)CC1]

In some embodiments, ring C is selected from those depicted in the compounds of Table 1 below.

As defined above and described herein, X¹ and X² are independently nitrogen or carbon.

In some embodiments, X¹ and X² are independently nitrogen or carbon. In some embodiments, at least one of X¹ and X² is nitrogen. In some embodiments, X¹ is nitrogen and X² is carbon. In some embodiments, X¹ is carbon and X² is nitrogen. In some embodiments, X¹ and X² are nitrogen.

In some embodiments, X¹ is selected from those depicted in the compounds of Table 1 below.

In some embodiments, X² is selected from those depicted in the compounds of Table 1 below.

As defined above and described herein, ring D is a ring selected from phenyl, a 4-7 membered saturated or partially unsaturated carbocyclyl, a 4-7 membered saturated or partially unsaturated heterocyclyl with 1-3 heteroatoms independently selected from nitrogen, oxygen, and sulfur, and a 5-6 membered heteroaryl with 1-4 heteroatoms independently selected from nitrogen, oxygen and sulfur.

In some embodiments, ring D is a phenyl. In some embodiments, ring D is a 4-7 membered saturated or partially unsaturated carbocyclyl. In some embodiments, ring D is a 4-7 membered saturated or partially unsaturated heterocyclyl with 1-3 heteroatoms independently selected from nitrogen, oxygen, and sulfur. In some embodiments, ring D is and a 5-6 membered heteroaryl with 1-4 heteroatoms independently selected from nitrogen, oxygen and sulfur.

In some embodiments, ring D is selected from those depicted in the compounds of Table 1 below.

As defined above and described herein, ring W is a ring selected from phenylenyl, a 4-7 membered saturated or partially unsaturated carbocyclylenyl, a 4-7 membered saturated or partially unsaturated heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur.

As defined above and described herein, L¹ is a covalent bond or a bivalent, saturated or partially unsaturated, straight or branched C₁₋₅ hydrocarbon chain, wherein 0-3 methylene units of L¹, L² or L^{A} are independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, or —OC(O)NR—, —NRC(O)O—.

In some embodiments, L¹ is a covalent bond. In some embodiments, L¹ is a bivalent, saturated or partially unsaturated, straight or branched C₁₋₅ hydrocarbon chain, wherein 0-3 methylene units of L¹ are independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, or —OC(O)NR—, —NRC(O)O—.

In some embodiments, L¹ is —O—. In some embodiments, L¹ is —NR—. In some embodiments, L¹ is —S—. In some embodiments, L¹ is —OC(O)—. In some embodiments, L¹ is —C(O)O—. In some embodiments, L¹ is —C(O)—. In some embodiments, L¹ is —S(O)—. In some embodiments, L¹ is —S(O)₂—. In some embodiments, L¹ is —NRS(O)₂—. In some embodiments, L¹ is —S(O)₂NR—. In some embodiments, L¹ is —NRC(O)—. In some embodiments, L¹ is —C(O)NR—. In some embodiments, L¹ is or —OC(O)NR—. In some embodiments, L¹ is —NRC(O)O—.

In some embodiments, L¹ is selected from those depicted in the compounds of Table 1 below.

As defined above and described herein, L^{A} is a covalent bond or a bivalent, saturated or partially unsaturated, straight or branched C₁₋₅ hydrocarbon chain, wherein 0-3 methylene units of L^{A} are independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, or —OC(O)NR—, —NRC(O)O—.

In some embodiments, L^{A} is a covalent bond. In some embodiments, L^{A} is a bivalent, saturated or partially unsaturated, straight or branched C₁₋₅ hydrocarbon chain, wherein 0-3 methylene units of L^{A} are independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, or —OC(O)NR—, —NRC(O)O—.

In some embodiments, L^{A} is —O—. In some embodiments, L^{A} is —NR—. In some embodiments, L^{A} is —S—. In some embodiments, L^{A} is —OC(O)—. In some embodiments, L^{A} is —C(O)O—. In some embodiments, L^{A} is —C(O)—. In some embodiments, L^{A} is —S(O)—. In some embodiments, L^{A} is —S(O)₂—. In some embodiments, L^{A} is —NRS(O)₃—. In some embodiments, L^{A} is —S(O)₂NR—. In some embodiments, L^{A} is —NRC(O)—. In some embodiments, L^{A} is —C(O)NR—. In some embodiments, L^{A} is or —OC(O)NR—. In some embodiments, L^{A} is —NRC(O)O—.

In some embodiments, L^{A} is —CH₂—.

In some embodiments, L^{A} is selected from those depicted in the compounds of Table 1 below.

As defined above and described herein, each R^{a} is independently selected from hydrogen, R^{A}, halogen, —CN, —NO₂, —OR, —SR, —NR₂, —S(O)₂R, —S(O)₂NR₂, —S(O)R, —C(O)R, —C(O)OR, —C(O)NR₂, —C(O)NROR, —OC(O)R, —OC(O)NR₂, —NRC(O)OR, —NRC(O)R, —NRC(O)N(R)₂, and —NRS(O)₂R.

In some embodiments, one or more of R^{a} is hydrogen. In some embodiments, one or more of R^{a} is R^{A}. In some embodiments, one or more of R^{a} is halogen. In some embodiments, one or more of R^{a} is —CN. In some embodiments, one or more of R^{a} is —NO₂. In some embodiments, one or more of R^{a} is —OR. In some embodiments, one or more of R^{a} is —SR. In some embodiments, one or more of R^{a} is —NR₂. In some embodiments, one or more of R^{a} is —S(O)₂R. In some embodiments, one or more of R^{a} is —S(O)₂NR In some embodiments, one or more of R^{a} is —S(O)R. In some embodiments, one or more of R^{a} is —C(O)R. In some embodiments, one or more of R^{a} is —C(O)OR. In some embodiments, one or more of R^{a} is —C(O)NR₂. In some embodiments, one or more of R^{a} is —C(O)NROR. In some embodiments, one or more of R^{a} is —OC(O)R. In some embodiments, one or more of R^{a} is —OC(O)NR₂. In some embodiments, one or more of R^{a} is —NRC(O)OR. In some embodiments, one or more of R^{a} is —NRC(O)R. In some embodiments, one or more of R^{a} is —NRC(O)N(R)₂. In some embodiments, one or more of R^{a} is —NRS(O)₂R.

In some embodiments, one or more R^{a} is —OH. In some embodiments, one or more R^{a} is methyl.

In some embodiments, R^{a} is selected from those depicted in the compounds of Table 1 below.

As defined above and described herein, each R^{b} is independently selected from hydrogen, R^{A}, halogen, —CN, —NO₂, —OR, —SR, —NR₂, —S(O)₂R, —S(O)₂NR₂, —S(O)R, —C(O)R, —C(O)OR, —C(O)NR₂, —C(O)NROR, —OC(O)R, —OC(O)NR₂, —NRC(O)OR, —NRC(O)R, —NRC(O)N(R)₂, and —NRS(O)₂R.

In some embodiments, one or more of R^{b} is hydrogen. In some embodiments, one or more of R^{b} is R^{A}. In some embodiments, one or more of R^{b} is halogen. In some embodiments, one or more of R^{b} is —CN. In some embodiments, one or more of R^{b} is —NO₂. In some embodiments, one or more of R^{b} is —OR. In some embodiments, one or more of R^{b} is —SR. In some embodiments, one or more of R^{b} is —NR₂. In some embodiments, one or more of R^{b} is —S(O)₂R. In some embodiments, one or more of R^{b} is —S(O)₂NR₂.

In some embodiments, one or more of R^{b} is —S(O)R. In some embodiments, one or more of R^{b} is —C(O)R. In some embodiments, one or more of R^{b} is —C(O)OR. In some embodiments, one or more of R^{b} is —C(O)NR₂. In some embodiments, one or more of R^{b} is —C(O)NROR. In some embodiments, one or more of R^{b} is —OC(O)R. In some embodiments, one or more of R^{b} is —OC(O)NR₂. In some embodiments, one or more of R^{b} is —NRC(O)OR. In some embodiments, one or more of R^{b} is —NRC(O)R. In some embodiments, one or more of R^{b} is —NRC(O)N(R)₂. In some embodiments, one or more of R^{b} is —NRS(O)₂R.

In some embodiments, R^{b} is selected from those depicted in the compounds of Table 1 below.

As defined above and described herein, each R^{e} is independently selected from hydrogen, R^{A}, halogen, —CN, —NO₂, —OR, —SR, —NR₂, —S(O)₂R, —S(O)₂NR₂, —S(O)R, —C(O)R, —C(O)OR, —C(O)NR₂, —C(O)NROR, —OC(O)R, —OC(O)NR₂, —NRC(O)OR, —NRC(O)R, —NRC(O)N(R)₂, and —NRS(O) ¿R.

In some embodiments, one or more of R^{c} is hydrogen. In some embodiments, one or more of R^{c} is R^{A}. In some embodiments, one or more of R^{c} is halogen. In some embodiments, one or more of R^{c} is —CN. In some embodiments, one or more of R^{c} is —NO₂. In some embodiments, one or more of R^{c} is —OR. In some embodiments, one or more of R^{c} is —SR. In some embodiments, one or more of R^{c} is —NR₂. In some embodiments, one or more of R^{◯} is —S(O)₂R. In some embodiments, one or more of R^{c} is —S(O)₂NR₂. In some embodiments, one or more of R^{c} is —S(O)R. In some embodiments, one or more of R^{c} is —C(O)R. In some embodiments, one or more of R^{c} is —C(O)OR. In some embodiments, one or more of R^{c} is —C(O)NR₂. In some embodiments, one or more of R^{c} is —C(O)NROR. In some embodiments, one or more of R^{c} is —OC(O)R. In some embodiments, one or more of R^{c} is —OC(O)NR₂. In some embodiments, one or more of R^{c} is —NRC(O)OR. In some embodiments, one or more of R^{c} is —NRC(O)R. In some embodiments, one or more of R^{c} is —NRC(O)N(R)₂. In some embodiments, one or more of R^{c} is —NRS(O)₂R.

In some embodiments, R^{c} is selected from those depicted in the compounds of Table 1 below.

As defined above and described herein, each R^{d} is independently selected from hydrogen, R^{A}, halogen, —CN, —NO₂, —OR, —SR, —NR₂, —S(O)₂R, —S(O)₂NR₂, —S(O)R, —C(O)R, —C(O)OR, —C(O)NR₂, —C(O)NROR, —OC(O)R, —OC(O)NR₂, —NRC(O)OR, —NRC(O)R, —NRC(O)N(R)₂, and —NRS(O)₂R.

In some embodiments, one or more of R^{d} is hydrogen. In some embodiments, one or more of R^{d} is R^{A}. In some embodiments, one or more of R^{d} is halogen. In some embodiments, one or more of R^{d} is —CN. In some embodiments, one or more of R^{d} is —NO₂. In some embodiments, one or more of R^{d} is —OR. In some embodiments, one or more of R^{d} is —SR. In some embodiments, one or more of R^{d} is —NR₂. In some embodiments, one or more of R^{d} is —S(O)₂R. In some embodiments, one or more of R^{d} is —S(O)₂NR. In some embodiments, one or more of R^{d} is —S(O)R. In some embodiments, one or more of R^{d} is —C(O)R. In some embodiments, one or more of R^{d} is —C(O)OR. In some embodiments, one or more of R^{d} is —C(O)NR₂. In some embodiments, one or more of R^{d} is —C(O)NROR. In some embodiments, one or more of R^{d} is —OC(O)R. In some embodiments, one or more of R^{d} is —OC(O)NR₂. In some embodiments, one or more of R^{d} is —NRC(O)OR. In some embodiments, one or more of R^{d} is —NRC(O)R. In some embodiments, one or more of R^{d} is —NRC(O)N(R)₂. In some embodiments, one or more of R^{d} is —NRS(O)₂R.

In some embodiments, one or more of R^{d} is halogen. In some embodiments, one or more of R^{d} is chloro.

In some embodiments, R^{d} is selected from those depicted in the compounds of Table 1 below.

As defined above and described herein, each R^{A} is independently an optionally substituted group selected from C₁₋₆ aliphatic, phenyl, a 3-12 membered saturated or partially unsaturated monocyclic, bicyclic, bridged bicyclic or spirocyclic carbocyclyl or heterocyclyl with 1-3 heteroatoms independently selected from nitrogen, oxygen, and sulfur, and a 5-6 membered heteroaryl ring having 1-4 heteroatoms independently selected from nitrogen, oxygen, and sulfur.

In some embodiments, R^{A} is an optionally substituted C₁₋₆ aliphatic. In some embodiments. R^{A} is a phenyl. In some embodiments, R^{A} is a 3-12 membered saturated or partially unsaturated monocyclic. In some embodiments, R^{A} is an optionally substituted bicyclic. In some embodiments, R^{A} is an optionally substituted bridged bicyclic or spirocyclic carbocyclyl. In some embodiments, R^{A} is a heterocyclyl with 1-3 heteroatoms independently selected from nitrogen, oxygen, and sulfur. In some embodiments, R^{A} is an optionally substituted 5-6 membered heteroaryl ring having 1-4 heteroatoms independently selected from nitrogen, oxygen, and sulfur.

In some embodiments, R^{A} is selected from those depicted in the compounds of Table 1 below.

As defined above and described herein, each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or two R groups on the same nitrogen are optionally take, together with their intervening atoms to form a 4-7 membered saturated, partially unsaturated, or heteroaryl ring having 0-3 heteroatoms, in addition to the nitrogen atom to which they are attached, independently selected from nitrogen, oxygen, and sulfur.

In some embodiments, one or more of R is hydrogen. In some embodiments, one or more of R is a C₁₋₆ aliphatic. In some embodiments, one or more of R is phenyl. In some embodiments, one or more of R is a 4-7 membered saturated or partially unsaturated heterocyclic having 1-2 heteroatoms independently selected from nitrogen, oxygen and sulfur. In some embodiments, one or more of R is and a 5-6 membered heteroaryl ring having 1-4 heteroatoms independently selected from nitrogen, oxygen, and sulfur. In some embodiments, two R groups on the same nitrogen are optionally taken together with their intervening atoms to form a 4-7 membered saturated, partially unsaturated, or heteroaryl ring having 0-3 heteroatoms, in addition to the nitrogen atom to which they are attached, independently selected from nitrogen, oxygen, and sulfur.

In some embodiments, R is selected from those depicted in the compounds of Table 1 below.

As defined above and described herein, a is 0, 1, 2, 3, or 4. In some embodiments, a is 0. In some embodiments, a is 1. In some embodiments, a is 2. In some embodiments, a is 3. In some embodiments, a is 4.

In some embodiments, a is selected from those depicted in the compounds of Table 1 below.

As defined above and described herein, b is 0, 1, 2, 3, or 4. In some embodiments, b is 0. In some embodiments, b is 1. In some embodiments, b is 2. In some embodiments, b is 3. In some embodiments, b is 4.

In some embodiments, b is selected from those depicted in the compounds of Table 1 below.

As defined above and described herein, c is 0, 1, 2, 3, or 4. In some embodiments, c is 0. In some embodiments, c is 1. In some embodiments, c is 2. In some embodiments, c is 3. In some embodiments, c is 4.

In some embodiments, c is selected from those depicted in the compounds of Table 1 below.

As defined above and described herein, d is 0, 1, 2, 3, or 4. In some embodiments, d is 0. In some embodiments, d is 1. In some embodiments, d is 2. In some embodiments, d is 3. In some embodiments, d is 4.

In some embodiments, d is selected from those depicted in the compounds of Table 1 below.

In some embodiments, PBM is

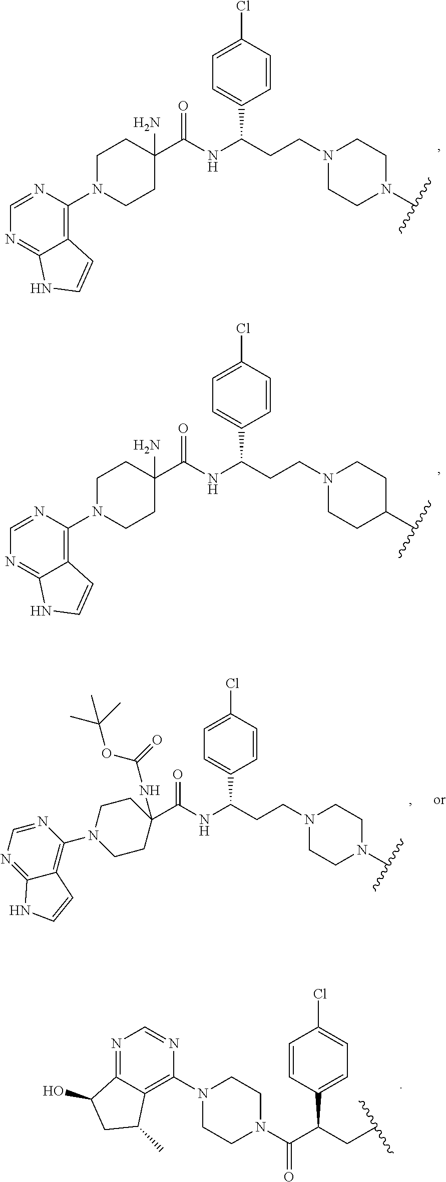 [SMILES: *N1CCN(CC[C@H](NC(=O)C2(N)CCN(c3ncnc4[nH]ccc34)CC2)c2ccc(Cl)cc2)CC1 ; *C1CCN(CC[C@H](NC(=O)C2(N)CCN(c3ncnc4[nH]ccc34)CC2)c2ccc(Cl)cc2)CC1 ; *N1CCN(CC[C@H](NC(=O)C2(NC(=O)OC(C)(C)C)CCN(c3ncnc4[nH]ccc34)CC2)c2ccc(Cl)cc2)CC1 ; *C[C@@H](C(=O)N1CCN(c2ncnc3c2[C@H](C)C[C@H]3O)CC1)c1ccc(Cl)cc1]

In some embodiments, PBM is

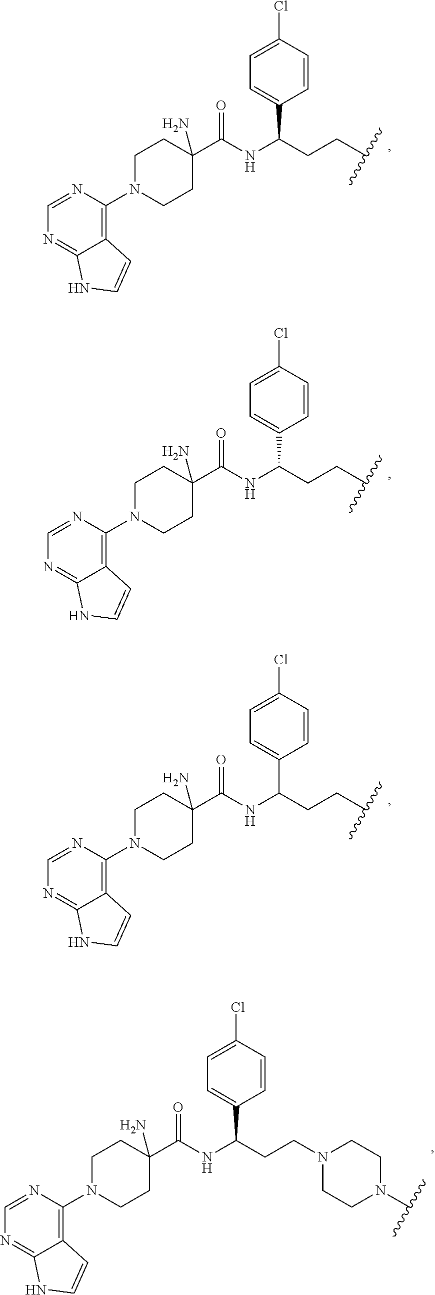 [SMILES: *CC[C@@H](NC(=O)C1(N)CCN(c2ncnc3[nH]ccc23)CC1)c1ccc(Cl)cc1 ; *CC[C@H](NC(=O)C1(N)CCN(c2ncnc3[nH]ccc23)CC1)c1ccc(Cl)cc1 ; *CCC(NC(=O)C1(N)CCN(c2ncnc3[nH]ccc23)CC1)c1ccc(Cl)cc1 ; *N1CCN(CC[C@@H](NC(=O)C2(N)CCN(c3ncnc4[nH]ccc34)CC2)c2ccc(Cl)cc2)CC1]

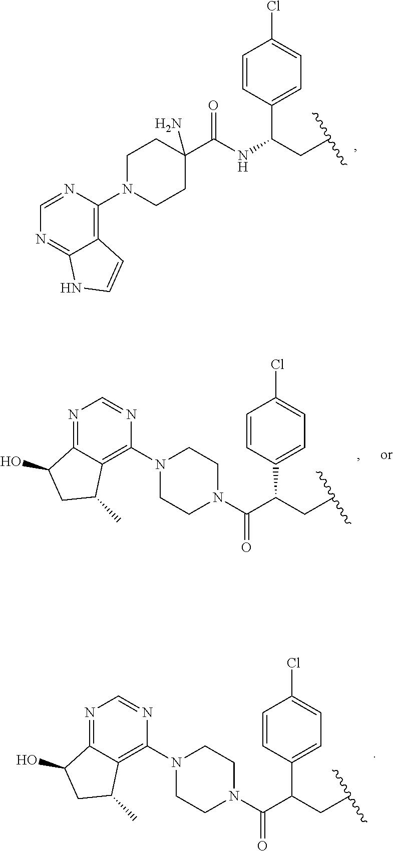 [SMILES: *C[C@H](NC(=O)C1(N)CCN(c2ncnc3[nH]ccc23)CC1)c1ccc(Cl)cc1 ; *C[C@H](C(=O)N1CCN(c2ncnc3c2[C@H](C)C[C@H]3O)CC1)c1ccc(Cl)cc1 ; *CC(C(=O)N1CCN(c2ncnc3c2[C@H](C)C[C@H]3O)CC1)c1ccc(Cl)cc1]

In some embodiments, PBM is

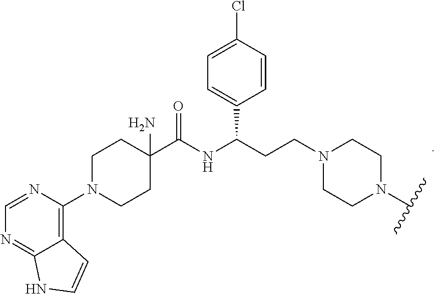 [SMILES: *N1CCN(CC[C@H](NC(=O)C2(N)CCN(c3ncnc4[nH]ccc34)CC2)c2ccc(Cl)cc2)CC1]

In some embodiments, PBM is

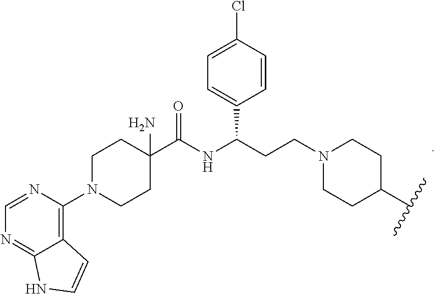 [SMILES: *C1CCN(CC[C@H](NC(=O)C2(N)CCN(c3ncnc4[nH]ccc34)CC2)c2ccc(Cl)cc2)CC1]

In some embodiments, PBM is

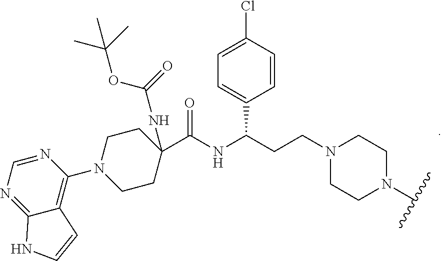 [SMILES: *N1CCN(CC[C@H](NC(=O)C2(NC(=O)OC(C)(C)C)CCN(c3ncnc4[nH]ccc34)CC2)c2ccc(Cl)cc2)CC1]

In some embodiments, PBM is

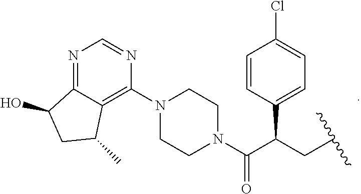 [SMILES: *C[C@@H](C(=O)N1CCN(c2ncnc3c2[C@H](C)C[C@H]3O)CC1)c1ccc(Cl)cc1]

In some embodiments, PBM is

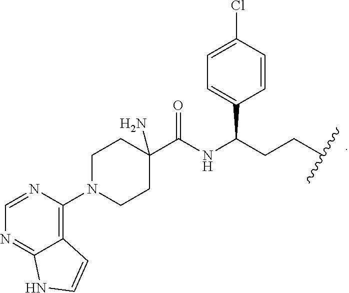 [SMILES: *CC[C@@H](NC(=O)C1(N)CCN(c2ncnc3[nH]ccc23)CC1)c1ccc(Cl)cc1]

In some embodiments. PBM is

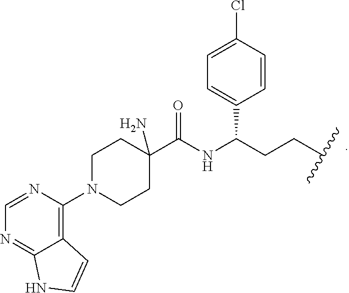 [SMILES: *CC[C@H](NC(=O)C1(N)CCN(c2ncnc3[nH]ccc23)CC1)c1ccc(Cl)cc1]

In some embodiments, PBM is

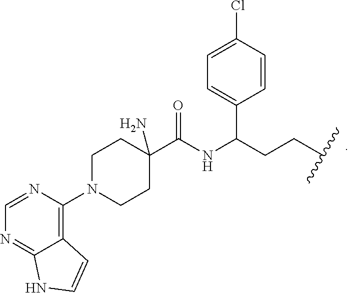 [SMILES: *CCC(NC(=O)C1(N)CCN(c2ncnc3[nH]ccc23)CC1)c1ccc(Cl)cc1]

In some embodiments, PBM is

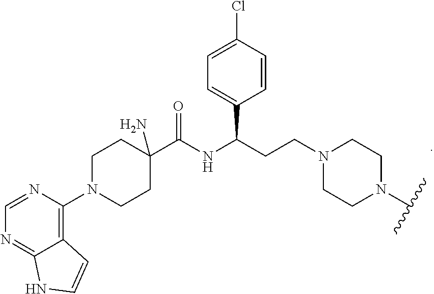 [SMILES: *N1CCN(CC[C@@H](NC(=O)C2(N)CCN(c3ncnc4[nH]ccc34)CC2)c2ccc(Cl)cc2)CC1]

In some embodiments, PBM is

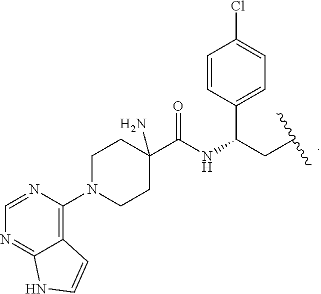 [SMILES: *C[C@H](NC(=O)C1(N)CCN(c2ncnc3[nH]ccc23)CC1)c1ccc(Cl)cc1]

In some embodiments, PBM is

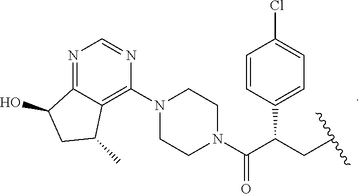 [SMILES: *C[C@H](C(=O)N1CCN(c2ncnc3c2[C@H](C)C[C@H]3O)CC1)c1ccc(Cl)cc1]

In some embodiments, PBM is selected from those depicted in Table 1, below.

In some embodiments, PBM is selected from those depicted in paragraph [0056], above.

### Deubiquitinase Binding Moiety (DBM)

In some embodiments, DIM is DBM. In some embodiments, DIM is a DUB ligand well known to one of ordinary skill in the art including those described in Caba et al., Biomolecules. 2022, 12 (5): 703; Harrigan et al., Nat Rev Drug Discov. 2018, 17 (1), 57-78; Lange et al., Mol Cell. 2022, 82 (1), 15-29; Pinto-Fernández et al., Front Chem. 2019, 7, 592; Schauer et al., J Med Chem. 2020, 63 (6), 2731-2750; and Wertz et al., Cell Chem Biol. 2019, 26 (2), 156-177, the entirety of each of which is hereby incorporated herein by reference.

In some embodiments, the DBM binds to OTUD1, OTUD7B, OTUB1, USP2, USP5, USP7, USP21, USP28, USP7, USP10, USP15, and/or AMSH. In some embodiments, the DBM binds to OTUD1. In some embodiments, the DBM binds to OTUD7B. In some embodiments, the DBM binds to OTUB1. In some embodiments, the DBM binds to USP2. In some embodiments, the DBM binds to USP5. In some embodiments, the DBM binds to USP7. In some embodiments, the DBM binds to USP21. In some embodiments, the DBM binds to USP28. In some embodiments, the DBM binds to USP7. In some embodiments, the DBM binds to USP10. In some embodiments, the DBM binds to USP15. In some embodiments, the DBM binds to AMSH.

As defined herein and described below, wherein a formula is depicted using square brackets, e.g.,

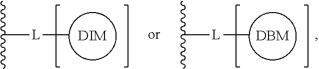

L is attached to a modifiable carbon, oxygen, or nitrogen atom within DIM or DBM including substitution or replacement of a defined group in DIM or DBM.

In some embodiments, the present invention provides a compound of formula I:

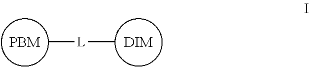

or a pharmaceutically acceptable salt thereof, wherein L and PBM are as described above and herein, and DIM is a deubiquitination inducing moiety, such as a deubiquitinase binding moiety (DBM).

In some embodiments, DIM is a DBM as described above and herein.

In some embodiments, the present invention provides a compound of formula I, wherein DBM is a deubiquitinase binding moiety thereby forming a compound of formula I-aa:

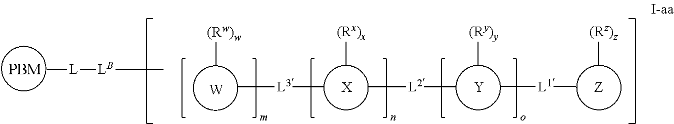

or a pharmaceutically acceptable salt thereof, wherein L and PBM are as defined above and described herein, and wherein:
Ring Z is a ring selected from phenyl, a 4-7 membered saturated or partially unsaturated carbocyclic ring, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, and an 8-10 membered bicyclic heteroaromatic ring having 1-5 heteroatoms independently selected from nitrogen, oxygen, or sulfur;
Ring Y is a ring selected from phenylenyl, a 4-7 membered saturated or partially unsaturated carbocyclylenyl, an 8-10 membered bicyclic aromatic carbocyclylenyl, a 4-8 membered saturated or partially unsaturated monocyclic heterocyclylenyl having 1-2 heteroatoms independently selected from nitrogen, oxygen, or sulfur, a 5-6 membered monocyclic heteroarylenyl having 1-4 heteroatoms independently selected from nitrogen, oxygen, or sulfur, and an 8-10 membered bicyclic heteroarylenyl having 1-5 heteroatoms independently selected from nitrogen, oxygen, or sulfur;
Ring X is a ring selected from phenylenyl, a 4-7 membered saturated or partially unsaturated carbocyclylenyl, a 4-7 membered saturated or partially unsaturated heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
Ring W is a ring selected from phenylenyl, a 4-7 membered saturated or partially unsaturated carbocyclylenyl, a 4-7 membered saturated or partially unsaturated heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
each of L^{B}, L^{1′}, L^{2′} and L^{3′} is independently a covalent bond or a bivalent, saturated or partially unsaturated, straight or branched C₁₋₅ hydrocarbon chain, wherein 0-3 methylene units of L^{B}, L^{1′}, L^{2′} or L^{3′} are independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, or —OC(O)NR—, —NRC(O)O—;
each R^{w}, R^{x}, R^{y}, and R^{z} is independently selected from hydrogen, R^{B}, halogen, —CN, —NO₂, —OR, —SR, —NR₂, —S(O)₂R, —S(O)₂NR₂, —S(O)R, —C(O)R, —C(O)OR, —C(O)NR₂, —C(O)NROR, —OC(O)R. —OC(O)NR₂, —NRC(O)OR, —NRC(O)R, —NRC(O)N(R)₂, and —NRS(O)₂R;
each R^{B} is independently an optionally substituted group selected from C₁₋₆ aliphatic, phenyl, a 3-12 membered saturated or partially unsaturated monocyclic, bicyclic, bridged bicyclic or spirocyclic carbocyclyl or heterocyclyl with 1-3 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or;
   two R groups on the same nitrogen are optionally taken together with their intervening atoms to form a 4-7 membered saturated, partially unsaturated, or heteroaryl ring having 0-3 heteroatoms, in addition to the nitrogen atom to which they are attached, independently selected from nitrogen, oxygen, and sulfur; and
m, n, and o are independently 0 or 1; and
w, x, y, and z are independently 0, 1, 2, 3, or 4.

In some embodiments, the present invention provides a compound of formula I-aa, wherein DBM is a deubiquitinase binding moiety thereby forming a compound of formula I-bb:

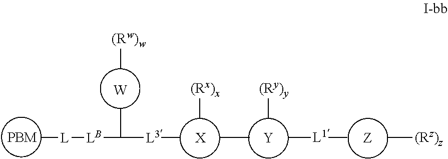

or a pharmaceutically acceptable salt thereof, wherein L and PBM are as defined above and described herein, and wherein:
Ring Z is a ring selected from phenyl, a 4-7 membered saturated or partially unsaturated carbocyclic ring, a 4-7 membered saturated or partially unsaturated heterocyclic ring with 1-3 heteroatoms independently selected from nitrogen, oxygen, and sulfur, and a 5-6 membered heteroaromatic ring with 1-4 heteroatoms independently selected from nitrogen, oxygen and sulfur;
Ring Y is a ring selected from phenylenyl, a 4-7 membered saturated or partially unsaturated carbocyclylenyl, a 4-7 membered saturated or partially unsaturated heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
Ring X is a ring selected from phenylenyl, a 4-7 membered saturated or partially unsaturated carbocyclylenyl, a 4-7 membered saturated or partially unsaturated heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
Ring W is a ring selected from phenylenyl, a 4-7 membered saturated or partially unsaturated carbocyclylenyl, a 4-7 membered saturated or partially unsaturated heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
each L^{B}, L^{1′}, and L^{3′} is independently a covalent bond or a bivalent, saturated or partially unsaturated, straight or branched C₁₋₅ hydrocarbon chain, wherein 0-3 methylene units of L^{B}, L^{1′}, or L^{3′} are independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, or —OC(O)NR—, —NRC(O)O—;
each R^{w}, R^{x}, R^{y}, and R^{z} is independently selected from hydrogen, R^{B}, halogen, —CN, —NO₂, —OR, —SR, —NR₂, —S(O)₂R, —S(O)₂NR₂, —S(O)R, —C(O)R, —C(O)OR, —C(O)NR₂, —C(O)NROR, —OC(O)R, —OC(O)NR₂, —NRC(O)OR, —NRC(O)R, —NRC(O)N(R)₂, and —NRS(O)₂R;
each R^{B} is independently an optionally substituted group selected from C₁₋₆ aliphatic, phenyl, a 3-12 membered saturated or partially unsaturated monocyclic, bicyclic, bridged bicyclic or spirocyclic carbocyclyl or heterocyclyl with 1-3 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or;
   two R groups on the same nitrogen are optionally taken together with their intervening atoms to form a 4-7 membered saturated, partially unsaturated, or heteroaryl ring having 0-3 heteroatoms, in addition to the nitrogen atom to which they are attached, independently selected from nitrogen, oxygen, and sulfur; and
w, x, y, and z are independently 0, 1, 2, 3, or 4.

In some embodiments, the present invention provides a compound of formula I, wherein DBM is a deubiquitinase binding moiety thereby forming a compound of formula I-bb:

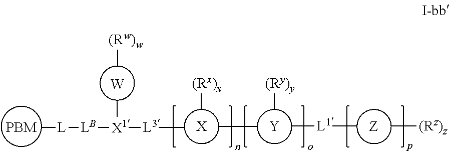

or a pharmaceutically acceptable salt thereof, wherein L and PBM are as defined above and described herein, and wherein:
Ring Z is a ring selected from phenyl, a 3-7 membered saturated or partially unsaturated monocyclic, bicyclic, bridged bicyclic or spirocyclic carbocyclic ring, a 3-7 membered saturated or partially unsaturated monocyclic, bicyclic, bridged bicyclic or spirocyclic heterocyclic ring with 1-3 heteroatoms independently selected from nitrogen, oxygen, and sulfur, and a 5-6 membered heteroaromatic ring with 1-4 heteroatoms independently selected from nitrogen, oxygen and sulfur;
Ring Y is a ring selected from phenylenyl, a 4-8 membered saturated or partially unsaturated carbocyclylenyl, a 4-8 membered saturated or partially unsaturated monocyclic, bicyclic, bridged bicyclic or spirocyclic heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
Ring X is a ring selected from phenylenyl, a 4-7 membered saturated or partially unsaturated carbocyclylenyl, a 4-7 membered saturated or partially unsaturated heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
Ring W is a ring selected from phenylenyl, a 4-7 membered saturated or partially unsaturated monocyclic, bicyclic, bridged bicyclic or spirocyclic carbocyclylenyl, a 4-7 membered saturated or partially unsaturated monocyclic, bicyclic, bridged bicyclic or spirocyclic heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
each L^{B}, L^{1′}, and L^{3′} is independently a covalent bond or an optionally substituted bivalent, saturated or partially unsaturated, straight or branched C₁₋₅ hydrocarbon chain, wherein 0-3 methylene units of L^{B}, L^{1′}, or L^{3′} are independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, or —OC(O)NR—, —NRC(O)O—;
each R^{w}, R^{x}, R^{y}, and R^{z} is independently selected from hydrogen, R^{B}, halogen, —CN, —NO₂, —OR, —SR, —NR₂, —S(O)₂R, —S(O)₂NR₂, —S(O)R, —C(O)R, —C(O)OR, —C(O)NR₂, —C(O)NROR, —OC(O)R. —OC(O)NR₂, —NRC(O)OR, —NRC(O)R, —NRC(O)N(R)₂, and —NRS(O)₂R;
each R^{B} is independently an optionally substituted group selected from C₁₋₆ aliphatic, phenyl, a 3-12 membered saturated or partially unsaturated monocyclic, bicyclic, bridged bicyclic or spirocyclic carbocyclyl or heterocyclyl with 1-3 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or;
   two R groups on the same nitrogen are optionally taken together with their intervening atoms to form a 4-7 membered saturated, partially unsaturated, or heteroaryl ring having 0-3 heteroatoms, in addition to the nitrogen atom to which they are attached, independently selected from nitrogen, oxygen, and sulfur;
   X^{1′} is carbon or nitrogen;
   m, n, o, and p are independently 0 or 1; and
w, x, y, and z are independently 0, 1, 2, 3, or 4.

In some embodiments, the present invention provides a compound of formula I-bb or I-bb′, wherein DBM is a deubiquitinase binding moiety thereby forming a compound of formula I-bb-1:

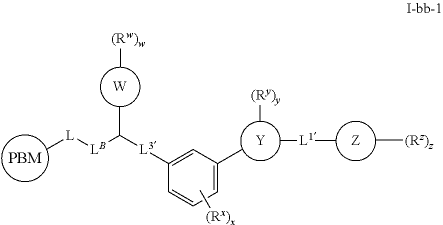

or a pharmaceutically acceptable salt thereof, wherein each of ring W, ring Y, ring Z, L^{B}, L^{3′}, L^{1′}, R^{w}, R^{x}, R^{y}, R^{z}, w, x, y, z, L, and PBM is as defined above and described in embodiments herein, both singly and in combination.

In some embodiments, the present invention provides a compound of formula I-bb or I-bb′, wherein DBM is a deubiquitinase binding moiety thereby forming a compound of formula I-bb-2:

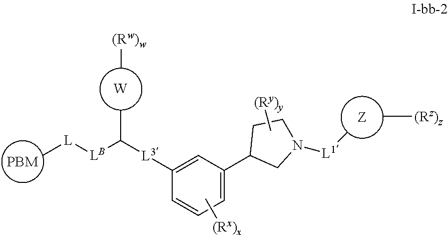

or a pharmaceutically acceptable salt thereof, wherein each of ring W, ring Z, L^{B}, L^{3′}, L^{1′}, R^{w}, R^{x}, R^{y}, R^{z}, w, x, y, z, L, and PBM is as defined above and described in embodiments herein, both singly and in combination.

In some embodiments, the present invention provides a compound of formula I-bb or I-bb′, wherein DBM is a deubiquitinase binding moiety thereby forming a compound of formula I-bb-3:

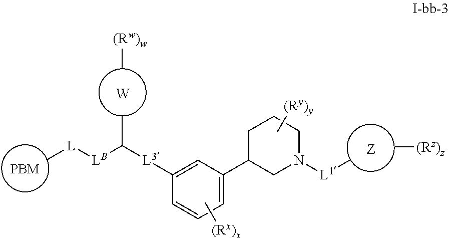

or a pharmaceutically acceptable salt thereof, wherein each of ring W, ring Z, L^{B}, L^{3′}, L^{1′}, R^{w}, R^{x}, R^{y}, R^{z}, w, x, y, z, L, and PBM is as defined above and described in embodiments herein, both singly and in combination.

In some embodiments, the present invention provides a compound of formula I-bb or I-bb′, wherein DBM is a deubiquitinase binding moiety thereby forming a compound of formula I-bb-4 or I-bb-4′:

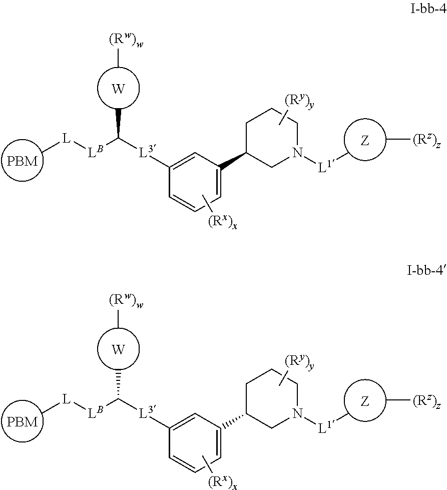

or a pharmaceutically acceptable salt thereof, wherein each of ring W, ring Z, L^{B}, L^{3′}, L^{1′}, R^{w}, R^{x}, R^{y}, R^{z}, w, x, y, z, L, and PBM is as defined above and described in embodiments herein, both singly and in combination.

In some embodiments, the present invention provides a compound of formula I-bb or I-bb′, wherein DBM is a deubiquitinase binding moiety thereby forming a compound of formula I-bb-5 or I-bb-5′:

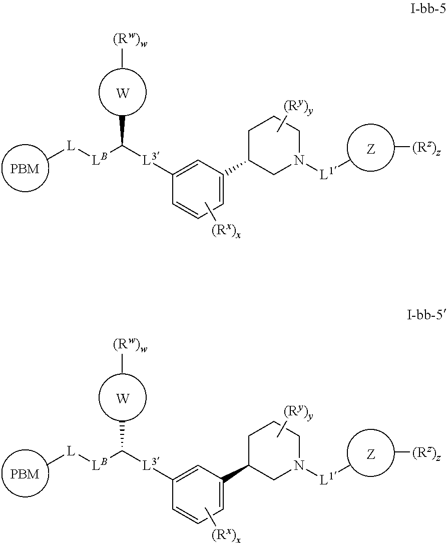

or a pharmaceutically acceptable salt thereof, wherein each of ring W, ring Z, L^{B}, L^{3′}, L^{1′}, R^{w}, R^{x}, R^{y}, R^{z}, w, x, y, z, L, and PBM is as defined above and described in embodiments herein, both singly and in combination.

In some embodiments, the present invention provides a compound of formula I-bb or I-bb′, wherein DBM is a deubiquitinase binding moiety thereby forming a compound of formula I-bb-6:

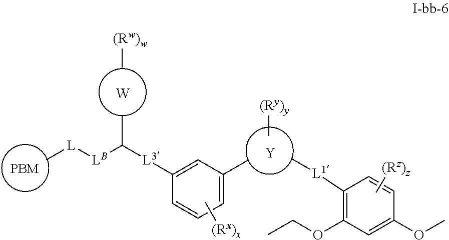

or a pharmaceutically acceptable salt thereof, wherein each of ring W, ring Y, L^{B}, L^{3′}, L^{1′}, R^{w}, R^{x}, R^{y}, R^{z}, w, x, y, z, L, and PBM is as defined above and described in embodiments herein, both singly and in combination.

In some embodiments, the present invention provides a compound of formula I-bb or I-bb′, wherein DBM is a deubiquitinase binding moiety thereby forming a compound of formula I-bb-7:

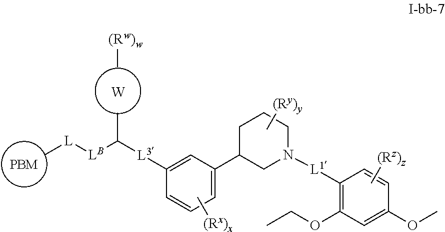

or a pharmaceutically acceptable salt thereof, wherein each of ring W, L^{B}, L^{3′}, L^{1′}, R^{w}, R^{x}, R^{y}, R^{z}, w, x, y, z, L, and PBM is as defined above and described in embodiments herein, both singly and in combination.

In some embodiments, the present invention provides a compound of formula I-bb or I-bb′, wherein DBM is a deubiquitinase binding moiety thereby forming a compound of formula I-bb-8:

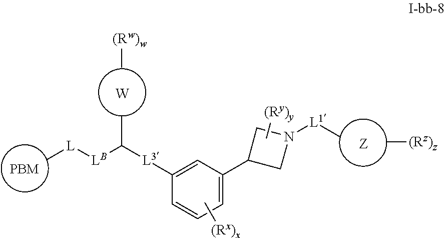

or a pharmaceutically acceptable salt thereof, wherein each of ring W, ring Z, L^{B}, L^{3′}, L^{1′}, R^{w}, R^{x}, R^{y}, R^{z}, w, x, y, z, L, and PBM is as defined above and described in embodiments herein, both singly and in combination.

In some embodiments, the present invention provides a compound of formula I-bb′, wherein DBM is a deubiquitinase binding moiety thereby forming a compound of formula I-bb′-1:

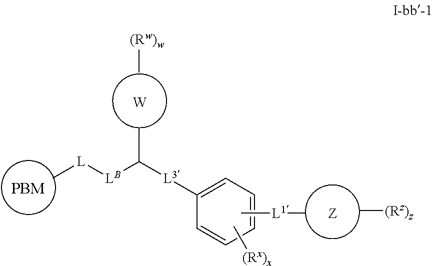

or a pharmaceutically acceptable salt thereof, wherein each of ring W, ring Z, L^{B}, L^{3′}, L^{1′}, R^{w}, R^{x}, R^{z}, w, x, z, L, and PBM is as defined above and described in embodiments herein, both singly and in combination

In some embodiments, the present invention provides a compound of formula I-bb′, wherein DBM is a deubiquitinase binding moiety thereby forming a compound of formula I-bb′-2:

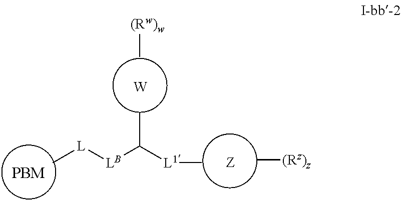

or a pharmaceutically acceptable salt thereof, wherein each of ring W, ring Z, L^{B}, L^{3′}, L^{1′}, R^{w}, R^{z}, w, z, L, and PBM is as defined above and described in embodiments herein, both singly and in combination.

In some embodiments, the present invention provides a compound of formula I-bb′, wherein DBM is a deubiquitinase binding moiety thereby forming a compound of formula I-bb′-3:

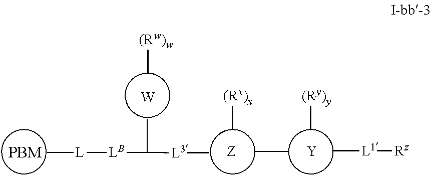

or a pharmaceutically acceptable salt thereof, wherein each of ring W, ring X, ring Y, L^{B}, L^{3′}, L^{1′}, R^{w}, R^{x}, R^{y}, R^{z}, w, x, y, L, and PBM is as defined above and described in embodiments herein, both singly and in combination.

In some embodiments, the present invention provides a compound of formula I-bb′, wherein DBM is a deubiquitinase binding moiety thereby forming a compound of formula I-bb′-4:

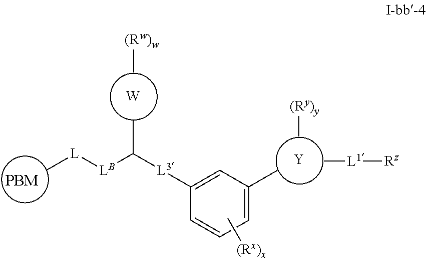

or a pharmaceutically acceptable salt thereof, wherein each of ring W, ring Y, L^{B}, L^{3′}, L^{1′}, R^{w}, R^{x}, R^{y}, R^{z}, w, x, y, L, and PBM is as defined above and described in embodiments herein, both singly and in combination.

In some embodiments, the present invention provides a compound of formula I-bb′, wherein DBM is a deubiquitinase binding moiety thereby forming a compound of formula I-bb′-5:

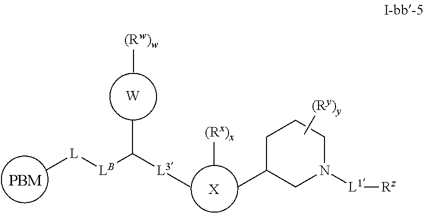

or a pharmaceutically acceptable salt thereof, wherein each of ring W, ring X, L^{B}, L^{3′}, L^{1′}, R^{w}, R^{x}, R^{y}, R^{z}, w, x, y, L, and PBM is as defined above and described in embodiments herein, both singly and in combination.

In some embodiments, the present invention provides a compound of formula I-bb′, wherein DBM is a deubiquitinase binding moiety thereby forming a compound of formula I-bb′-6:

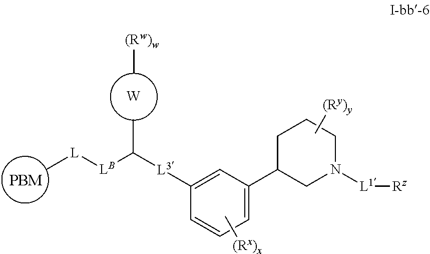

or a pharmaceutically acceptable salt thereof, wherein each of ring W, L^{B}, L^{3′}, L^{1′}, R^{w}, R^{x}, R^{y}, R^{z}, w, x, y, L, and PBM is as defined above and described in embodiments herein, both singly and in combination.

In some embodiments, the present invention provides a compound of formula I-aa, wherein DBM is a deubiquitinase binding moiety thereby forming a compound of formula I-cc:

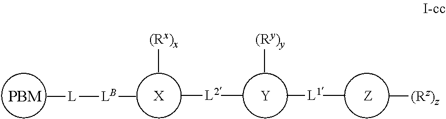

or a pharmaceutically acceptable salt thereof, wherein L and PBM are as defined above and described herein, and wherein:
Ring Z is a ring selected from phenyl, a 4-7 membered saturated or partially unsaturated carbocyclic ring, a 4-7 membered saturated or partially unsaturated heterocyclic ring with 1-3 heteroatoms independently selected from nitrogen, oxygen, and sulfur, and a 5-6 membered heteroaromatic ring with 1-4 heteroatoms independently selected from nitrogen, oxygen and sulfur;
Ring Y is a ring selected from phenylenyl, a 4-7 membered saturated or partially unsaturated carbocyclylenyl, a 4-7 membered saturated or partially unsaturated heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
Ring X is a ring selected from phenylenyl, a 4-7 membered saturated or partially unsaturated carbocyclylenyl, a 4-7 membered saturated or partially unsaturated heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
each of L^{B}, L^{1′}, and L^{2′} is independently a covalent bond or a bivalent, saturated or partially unsaturated, straight or branched C₁₋₅ hydrocarbon chain, wherein 0-3 methylene units of L^{B}, L^{1′}, or L^{2′} are independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, or —OC(O)NR—, —NRC(O)O—;
each R^{x}, R^{y}, and R^{z} is independently selected from hydrogen, R^{B}, halogen, CN, —NO₂, —OR, —SR, —NR₂, —S(O)₂R, —S(O)₂NR₂, —S(O)R, —C(O)R, —C(O)OR, —C(O)NR₂, —C(O)NROR, —OC(O)R. —OC(O)NR₂, —NRC(O)OR, —NRC(O)R, —NRC(O)N(R)₂, and —NRS(O)₂R;
each R^{B} is independently an optionally substituted group selected from C₁₋₆ aliphatic, phenyl, a 3-12 membered saturated or partially unsaturated monocyclic, bicyclic, bridged bicyclic or spirocyclic carbocyclyl or heterocyclyl with 1-3 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or;
   two R groups on the same nitrogen are optionally taken together with their intervening atoms to form a 4-7 membered saturated, partially unsaturated, or heteroaryl ring having 0-3 heteroatoms, in addition to the nitrogen atom to which they are attached, independently selected from nitrogen, oxygen, and sulfur; and
x, y, and z are independently 0, 1, 2, 3, or 4.

In some embodiments, the present invention provides a compound of formula I-aa, wherein DBM is a deubiquitinase binding moiety thereby forming a compound of formula I-cc′:

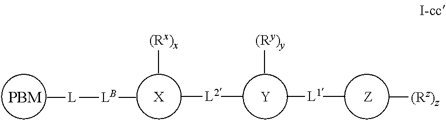

or a pharmaceutically acceptable salt thereof, wherein L and PBM are as defined above and described herein, and wherein:
Ring Z is a ring selected from phenyl, a 3-7 membered saturated or partially unsaturated monocyclic, bicyclic, bridged bicyclic or spirocyclic carbocyclic ring, a 3-7 membered saturated or partially unsaturated monocyclic, bicyclic, bridged bicyclic or spirocyclic heterocyclic ring with 1-3 heteroatoms independently selected from nitrogen, oxygen, and sulfur, and a 5-6 membered heteroaromatic ring with 1-4 heteroatoms independently selected from nitrogen, oxygen and sulfur and an 8-10 membered bicyclic heteroaromatic ring having 1-5 heteroatoms independently selected from nitrogen, oxygen, or sulfur;
Ring Y is a ring selected from phenylenyl, a 4-7 membered saturated or partially unsaturated carbocyclylenyl, a 4-8 membered saturated or partially unsaturated monocyclic, bicyclic, bridged bicyclic or spirocyclic heterocyclylenyl with 1-3 heteroatoms independently selected from nitrogen, oxygen, and sulfur, a 5-6 membered heteroarylenyl with 1-4 heteroatoms independently selected from nitrogen, oxygen and sulfur and an 8-10 membered bicyclic heterocyclylenyl having 1-5 heteroatoms independently selected from nitrogen, oxygen, or sulfur;
Ring X is a ring selected from phenylenyl, a 4-7 membered saturated or partially unsaturated carbocyclylenyl, a 4-7 membered saturated or partially unsaturated heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
each of L^{B}, L^{1′}, and L^{2′} is independently a covalent bond or an optionally substituted bivalent, saturated or partially unsaturated, straight or branched C₁₋₅ hydrocarbon chain, wherein 0-3 methylene units of L^{B}, L^{1′}, or L^{2′} are independently replaced by -Cy′-, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, or —OC(O)NR—, —NRC(O)O—;
each R^{x}, R^{y}, and R^{z} is independently selected from hydrogen, R^{B}, halogen, CN, —NO₂, —OR, —SR, —NR₂, —S(O)₂R, —S(O)₂NR₂, —S(O)R, —C(O)R, —C(O)OR, —C(O)NR₂, —C(O)NROR, —OC(O)R, —OC(O)NR₂, —NRC(O)OR, —NRC(O)R, —NRC(O)N(R)₂, and —NRS(O)₂R;
each R^{B} is independently an optionally substituted group selected from C₁₋₆ aliphatic, phenyl, a 3-12 membered saturated or partially unsaturated monocyclic, bicyclic, bridged bicyclic or spirocyclic carbocyclyl or heterocyclyl with 1-3 heteroatoms independently selected from nitrogen, oxygen, and sulfur, and a 5-6 membered heteroaryl ring having 1-4 heteroatoms independently selected from nitrogen, oxygen, and sulfur;
each -Cy′- is independently an optionally substituted bivalent ring selected from phenylenyl, an 8-10 membered bicyclic arylenyl, a 3-11 membered saturated or partially unsaturated monocyclic, bicyclic, bridged bicyclic or spirocyclic carbocyclylenyl, a 3-11 membered saturated or partially unsaturated monocyclic, bicyclic, bridged bicyclic or spirocyclic heterocyclylenyl having 1-3 heteroatoms independently selected from nitrogen, oxygen, and sulfur, a 5-6 membered heteroarylenyl having 1-4 heteroatoms independently selected from nitrogen, oxygen, and sulfur, and an 8-10 membered bicyclic heteroarylenyl having 1-5 heteroatoms independently selected from nitrogen, oxygen, and sulfur;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or;
   two R groups on the same nitrogen are optionally taken together with their intervening atoms to form a 4-7 membered saturated, partially unsaturated, or heteroaryl ring having 0-3 heteroatoms, in addition to the nitrogen atom to which they are attached, independently selected from nitrogen, oxygen, and sulfur; and
x, y, and z are independently 0, 1, 2, 3, or 4.

In some embodiments, the present invention provides a compound of formula I-cc, wherein DBM is a deubiquitinase binding moiety thereby forming a compound of formula I-cc-1:

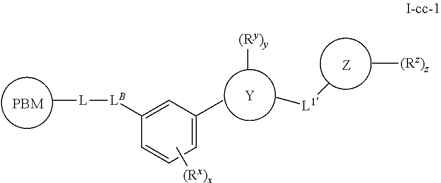

or a pharmaceutically acceptable salt thereof, wherein each of ring Y, ring Z, L^{B}, L^{1′}, R^{x}, R^{y}, R^{z}, x, y, z, L, and PBM is as defined above and described in embodiments herein, both singly and in combination.

In some embodiments, the present invention provides a compound of formula I-cc, wherein DBM is a deubiquitinase binding moiety thereby forming a compound of formula I-cc-2:

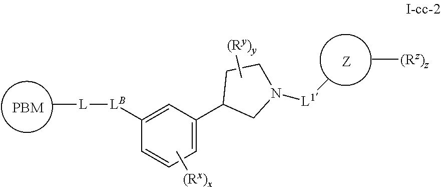

or a pharmaceutically acceptable salt thereof, wherein each of ring Z, L^{B}, L^{1′}, R^{x}, R^{y}, R^{z}, x, y, z, L, and PBM is as defined above and described in embodiments herein, both singly and in combination.

In some embodiments, the present invention provides a compound of formula I-cc, wherein DBM is a deubiquitinase binding moiety thereby forming a compound of formula I-cc-3 or I-cc-3′:

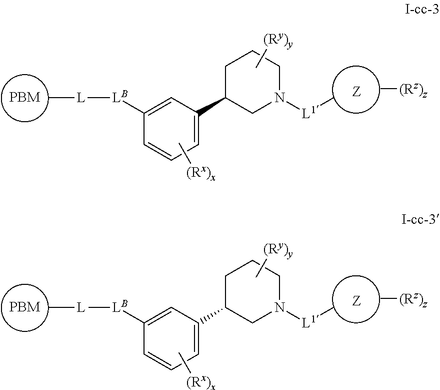

or a pharmaceutically acceptable salt thereof, wherein each of ring Z, L^{B}, L^{1′}, R^{x}, R^{y}, R^{z}, x, y, z, L, and PBM is as defined above and described in embodiments herein, both singly and in combination.

In some embodiments, the present invention provides a compound of formula I-cc, wherein DBM is a deubiquitinase binding moiety thereby forming a compound of formula I-cc-4:

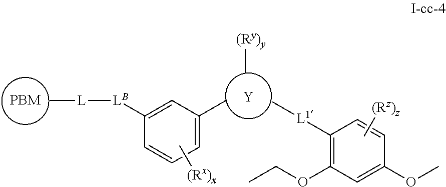

or a pharmaceutically acceptable salt thereof, wherein each of ring Y, L^{B}, L^{1′}, R^{x}, R^{y}, R^{z}, x, y, z, L, and PBM is as defined above and described in embodiments herein, both singly and in combination.

In some embodiments, the present invention provides a compound of formula I-cc, wherein DBM is a deubiquitinase binding moiety thereby forming a compound of formula I-cc-5:

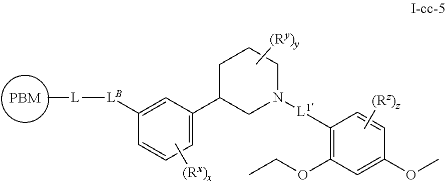

or a pharmaceutically acceptable salt thereof, wherein each of L^{B}, L^{1′}, R^{x}, R^{y}, R^{z}, x, y, z, L, and PBM is as defined above and described in embodiments herein, both singly and in combination.

In some embodiments, the present invention provides a compound of formula I-cc, wherein DBM is a deubiquitinase binding moiety thereby forming a compound of formula I-dd-1:

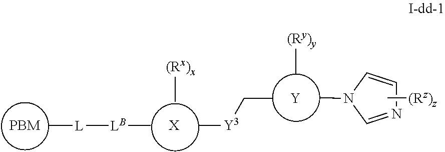

or a pharmaceutically acceptable salt thereof, wherein each of ring X, ring Y, L^{B}, R^{x}, R^{y}, R^{z}, x, y, z, L, and PBM is as defined above and described in embodiments herein, both singly and in combination, wherein Y³ is sulfur or oxygen.

In some embodiments, the present invention provides a compound of formula I-cc, wherein DBM is a deubiquitinase binding moiety thereby forming a compound of formula I-dd-2:

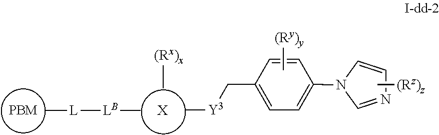

or a pharmaceutically acceptable salt thereof, wherein ring X, L^{B}, R^{x}, R^{y}, R^{z}, x, y, z, L, and PBM is as defined above and described in embodiments herein, both singly and in combination, wherein Y³ is sulfur or oxygen.

In some embodiments, the present invention provides a compound of formula I-cc, wherein DBM is a deubiquitinase binding moiety thereby forming a compound of formula I-dd-3 or I-dd-3′:

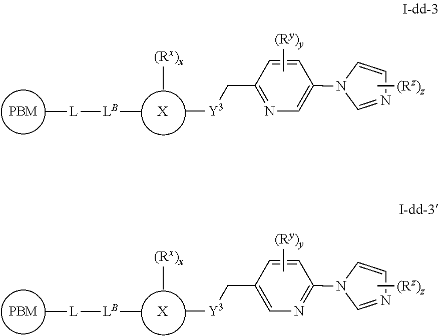

or a pharmaceutically acceptable salt thereof, wherein each of ring X, L^{B}, R^{x}, R^{y}, R^{z}, x, y, z, L, and PBM is as defined above and described in embodiments herein, both singly and in combination, wherein Y³ is sulfur or oxygen.

In some embodiments, the present invention provides a compound of formula I-cc, wherein DBM is a deubiquitinase binding moiety thereby forming a compound of formula I-dd-3:

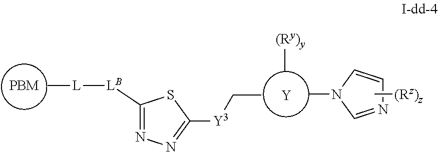

or a pharmaceutically acceptable salt thereof, wherein each of ring Y, L^{B}, R^{y}, R^{z}, y, z, L, and PBM is as defined above and described in embodiments herein, both singly and in combination, wherein Y³ is sulfur or oxygen.

In some embodiments, the present invention provides a compound of formula I-cc, wherein DBM is a deubiquitinase binding moiety thereby forming a compound of formula I-dd-5:

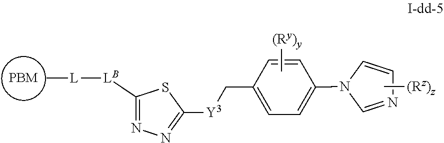

or a pharmaceutically acceptable salt thereof, wherein each of L^{B}, R^{y}, R^{z}, y, z, L, and PBM is as defined above and described in embodiments herein, both singly and in combination, wherein Y³ is sulfur or oxygen.

In some embodiments, the present invention provides a compound of formula I-cc, wherein DBM is a deubiquitinase binding moiety thereby forming a compound of formula I-ee-1:

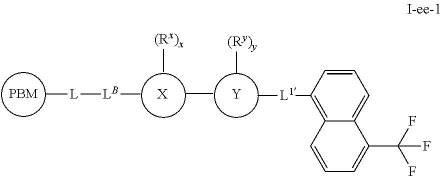

or a pharmaceutically acceptable salt thereof, wherein each of ring X, ring Y, L^{B}, L^{1′}, R^{x}, R^{y}, x, y, L, and PBM is as defined above and described in embodiments herein.

In some embodiments, the present invention provides a compound of formula I-cc, wherein DBM is a deubiquitinase binding moiety thereby forming a compound of formula I-ee-2:

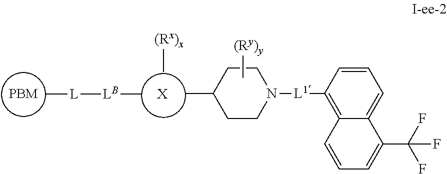

or a pharmaceutically acceptable salt thereof, wherein each of ring X, L^{B}, L^{1′}, R^{x}, R^{y}, x, y, L, and PBM is as defined above and described in embodiments herein.

In some embodiments, the present invention provides a compound of formula I-cc, wherein DBM is a deubiquitinase binding moiety thereby forming a compound of formula I-ee-3:

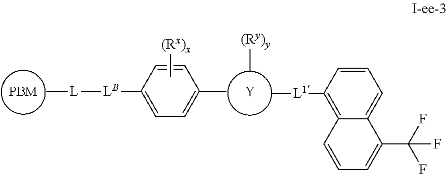

or a pharmaceutically acceptable salt thereof, wherein each of ring Y, L^{B}, L^{1′}, R^{x}, R^{y}, x, y, L, and PBM is as defined above and described in embodiments herein.

In some embodiments, the present invention provides a compound of formula I-cc′, wherein DBM is a deubiquitinase binding moiety thereby forming a compound of formula I-cc′-1:

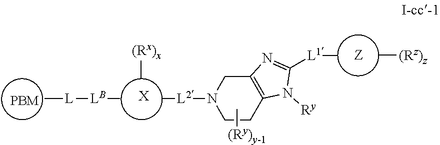

or a pharmaceutically acceptable salt thereof, wherein each of ring X, ring Z, L^{B}, L^{1′}, L^{2′}, R^{x}, R^{y}, R^{z}, x, y, z, L, and PBM is as defined above and described in embodiments herein.

In some embodiments, the present invention provides a compound of formula I-cc′, wherein DBM is a deubiquitinase binding moiety thereby forming a compound of formula I-cc′-2:

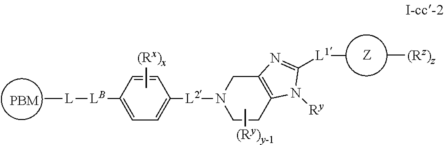

or a pharmaceutically acceptable salt thereof, wherein each of ring Z, L^{B}, L^{1′}, L^{2′}, R^{x}, R^{y}, R^{z}, x, y, z, L, and PBM is as defined above and described in embodiments herein.

In some embodiments, the present invention provides a compound of formula I-aa, wherein DBM is a deubiquitinase binding moiety thereby forming a compound of formula I-ff:

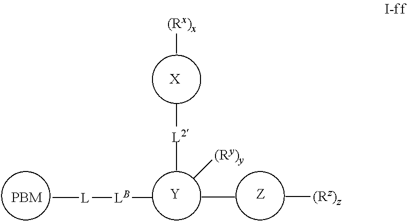

or a pharmaceutically acceptable salt thereof, wherein L and PBM are as defined above and described herein, and wherein:
Ring Z is a ring selected from phenyl, a 4-7 membered saturated or partially unsaturated carbocyclic ring, a 4-7 membered saturated or partially unsaturated heterocyclic ring with 1-3 heteroatoms independently selected from nitrogen, oxygen, and sulfur, and a 5-6 membered heteroaromatic ring with 1-4 heteroatoms independently selected from nitrogen, oxygen and sulfur;
Ring Y is a ring selected from phenylenyl, a 4-7 membered saturated or partially unsaturated carbocyclylenyl, a 4-7 membered saturated or partially unsaturated heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
Ring X is a ring selected from phenylenyl, a 4-7 membered saturated or partially unsaturated carbocyclylenyl, a 4-7 membered saturated or partially unsaturated heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
each of L^{B} and L¹ is independently a covalent bond or a bivalent, saturated or partially unsaturated, straight or branched C₁₋₅ hydrocarbon chain, wherein 0-3 methylene units of L^{B} or L^{2′} are independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, or —OC(O)NR—, —NRC(O)O—;
each R^{x}, R^{y}, and R^{z} is independently selected from hydrogen, R^{B}, halogen, —CN, —NO₂, —OR, —SR, —NR₂, —S(O)₂R, —S(O)₂NR₂, —S(O)R, —C(O)R, —C(O)OR, —C(O)NR₂, —C(O)NROR, —OC(O)R, —OC(O)NR₂, —NRC(O)OR, —NRC(O)R, —NRC(O)N(R)₂, and —NRS(O)₂R;
each R^{B} is independently an optionally substituted group selected from C₁₋₆ aliphatic, phenyl, a 3-12 membered saturated or partially unsaturated monocyclic, bicyclic, bridged bicyclic or spirocyclic carbocyclyl or heterocyclyl with 1-3 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or;
   two R groups on the same nitrogen are optionally taken together with their intervening atoms to form a 4-7 membered saturated, partially unsaturated, or heteroaryl ring having 0-3 heteroatoms, in addition to the nitrogen atom to which they are attached, independently selected from nitrogen, oxygen, and sulfur; and
x, y, and z are independently 0, 1, 2, 3, or 4.

In some embodiments, the present invention provides a compound of formula I-aa, wherein DBM is a deubiquitinase binding moiety thereby forming a compound of formula I-ff′:

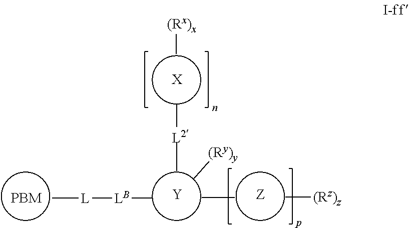

or a pharmaceutically acceptable salt thereof, wherein L and PBM are as defined above and described herein, and wherein:
Ring Z is a ring selected from phenyl, a 4-9 membered saturated or partially unsaturated monocyclic, bicyclic, bridged bicyclic or spirocyclic carbocyclic ring, a 4-9 membered saturated or partially unsaturated monocyclic, bicyclic, bridged bicyclic or spirocyclic heterocyclic ring with 1-3 heteroatoms independently selected from nitrogen, oxygen, and sulfur, a 5-6 membered heteroaromatic ring with 1-4 heteroatoms independently selected from nitrogen, oxygen and sulfur and an 8-10 membered bicyclic heteroaromatic ring having 1-5 heteroatoms independently selected from nitrogen, oxygen, or sulfur;
Ring Y is a ring selected from phenylenyl, a 4-7 membered saturated or partially unsaturated carbocyclylenyl, a 4-7 membered saturated or partially unsaturated heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
Ring X is a ring selected from phenylenyl, a 3-9 membered saturated or partially unsaturated monocyclic, bicyclic, bridged bicyclic or spirocyclic carbocyclylenyl, a 3-9 membered saturated or partially unsaturated monocyclic, bicyclic, bridged bicyclic or spirocyclic heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
each of L^{B} and L¹ is independently a covalent bond or an optionally substituted bivalent, saturated or partially unsaturated, straight or branched C₁₋₅ hydrocarbon chain, wherein 0-3 methylene units of L^{B} or L^{2′} are independently replaced by -Cy′-, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, or —OC(O)NR—, —NRC(O)O—;
each R^{x}, R^{y}, and R^{z} is independently selected from hydrogen, R^{B}, halogen, —CN, —NO₂, —OR, —SR, —NR₂, —S(O)₂R, —S(O)₂NR₂, —S(O)R, —C(O)R, —C(O)OR, —C(O)NR₂, —C(O)NROR, —OC(O)R. —OC(O)NR₂, —NRC(O)OR, —NRC(O)R, —NRC(O)N(R)₂, and —NRS(O)₂R;
each R^{B} is independently an optionally substituted group selected from C₁₋₆ aliphatic, phenyl, a 3-12 membered saturated or partially unsaturated monocyclic, bicyclic, bridged bicyclic or spirocyclic carbocyclyl or heterocyclyl with 1-3 heteroatoms independently selected from nitrogen, oxygen, and sulfur, and a 5-6 membered heteroaryl ring having 1-4 heteroatoms independently selected from nitrogen, oxygen, and sulfur;
each -Cy′- is independently an optionally substituted bivalent ring selected from phenylenyl, an 8-10 membered bicyclic arylenyl, a 3-11 membered saturated or partially unsaturated monocyclic, bicyclic, bridged bicyclic or spirocyclic carbocyclylenyl, a 3-11 membered saturated or partially unsaturated monocyclic, bicyclic, bridged bicyclic or spirocyclic heterocyclylenyl having 1-3 heteroatoms independently selected from nitrogen, oxygen, and sulfur, a 5-6 membered heteroarylenyl having 1-4 heteroatoms independently selected from nitrogen, oxygen, and sulfur, and an 8-10 membered bicyclic heteroarylenyl having 1-5 heteroatoms independently selected from nitrogen, oxygen, and sulfur;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or;
   two R groups on the same nitrogen are optionally taken together with their intervening atoms to form a 4-7 membered saturated, partially unsaturated, or heteroaryl ring having 0-3 heteroatoms, in addition to the nitrogen atom to which they are attached, independently selected from nitrogen, oxygen, and sulfur;
n and p are independently 0 or 1; and
x, y, and z are independently 0, 1, 2, 3, or 4.

In some embodiments, the present invention provides a compound of formula I-ff or I-ff′, wherein DBM is a deubiquitinase binding moiety thereby forming a compound of formula I-ff-2:

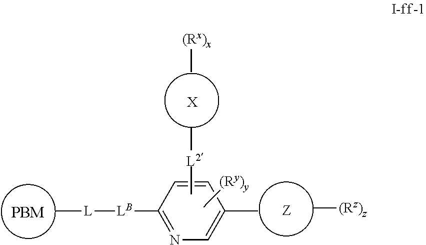

or a pharmaceutically acceptable salt thereof, wherein each of ring X, ring Z, L^{B}, L^{2′}, R^{x}, R^{y}, R^{z}, x, y, z, L, and PBM is as defined above and described in embodiments herein, both singly and in combination.

In some embodiments, the present invention provides a compound of formula I-ff or I-ff′, wherein DBM is a deubiquitinase binding moiety thereby forming a compound of formula I-ff-2′:

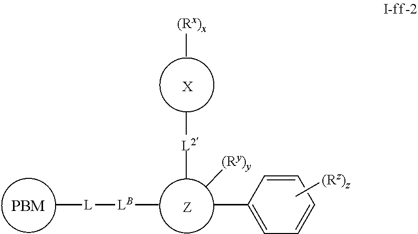

or a pharmaceutically acceptable salt thereof, wherein each of ring X, ring Y, L^{B}, L^{2′}, R^{x}, R^{y}, R^{z}, x, y, z, L, and PBM is as defined above and described in embodiments herein, both singly and in combination.

In some embodiments, the present invention provides a compound of formula I-ff or I-ff′, wherein DBM is a deubiquitinase binding moiety thereby forming a compound of formula I-ff-3:

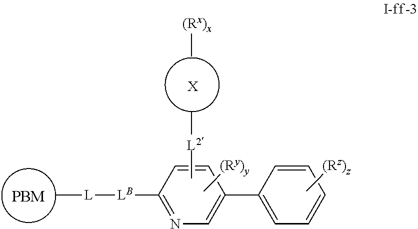

or a pharmaceutically acceptable salt thereof, wherein each of ring X, L^{B}, L^{2′}, R^{x}, R^{y}, R^{z}, x, y, z, L, and PBM is as defined above and described in embodiments herein, both singly and in combination.

In some embodiments, the present invention provides a compound of formula I-ff or I-ff′, wherein DBM is a deubiquitinase binding moiety thereby forming a compound of formula I-ff-4:

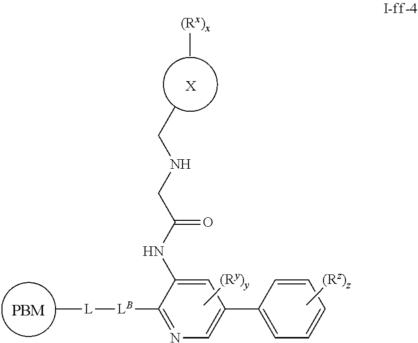

or a pharmaceutically acceptable salt thereof, wherein each of ring X, L^{B}, L^{2′}, R^{x}, R^{y}, R^{z}, x, y, z, L, and PBM is as defined above and described in embodiments herein, both singly and in combination.

In some embodiments, the present invention provides a compound of formula I-ff′, wherein DBM is a deubiquitinase binding moiety thereby forming a compound of formula I-ff′-1:

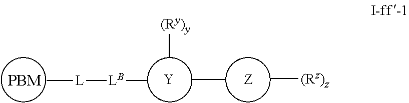

or a pharmaceutically acceptable salt thereof, wherein each of ring Y, ring Z, L^{B}, R^{y}, R^{z}, y, z, L, and PBM is as defined above and described in embodiments herein, both singly and in combination.

In some embodiments, the present invention provides a compound of formula I-ff′, wherein DBM is a deubiquitinase binding moiety thereby forming a compound of formula I-ff′-2:

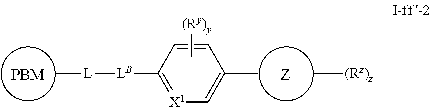

or a pharmaceutically acceptable salt thereof, wherein each of ring Y, ring Z, L^{B}, R^{y}, R^{z}, y, z, L, and PBM is as defined above and described in embodiments herein, both singly and in combination; and wherein X¹ is carbon or nitrogen.

In some embodiments, the present invention provides a compound of formula I-ff′, wherein DBM is a deubiquitinase binding moiety thereby forming a compound of formula I-ff′-3:

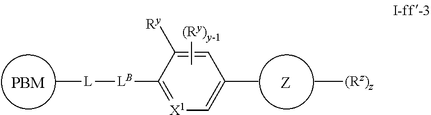

or a pharmaceutically acceptable salt thereof, wherein each of ring Z, L^{B}, R^{y}, R^{z}, y, z, L, and PBM is as defined above and described in embodiments herein, both singly and in combination.

In some embodiments, the present invention provides a compound of formula I-ff′, wherein DBM is a deubiquitinase binding moiety thereby forming a compound of formula I-ff′-4:

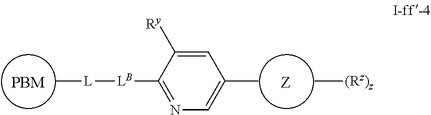

or a pharmaceutically acceptable salt thereof, wherein each of ring Z, L^{B}, R^{y}, R^{z}, y, z, L, and PBM is as defined above and described in embodiments herein, both singly and in combination.

In some embodiments, the present invention provides a compound of formula I-ff′, wherein DBM is a deubiquitinase binding moiety thereby forming a compound of formula I-ff′-5:

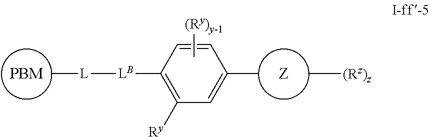

or a pharmaceutically acceptable salt thereof, wherein each of ring Z, L^{B}, R^{y}, R^{z}, y, z, L, and PBM is as defined above and described in embodiments herein, both singly and in combination. In some embodiments, the present invention provides a compound of formula I-ff′, wherein DBM is a deubiquitinase binding moiety thereby forming a compound of formula I-ff′-6:

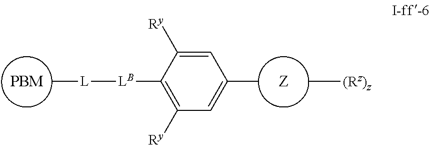

or a pharmaceutically acceptable salt thereof, wherein each of ring Z, L^{B}, R^{y}, R^{z}, y, z, L, and PBM is as defined above and described in embodiments herein, both singly and in combination.

In some embodiments, the present invention provides a compound of formula I-aa, wherein DBM is a deubiquitinase binding moiety thereby forming a compound of formula I-gg:

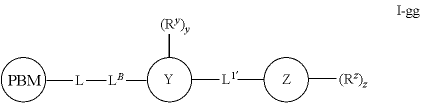

or a pharmaceutically acceptable salt thereof, wherein L and PBM are as defined above and described herein, and wherein:
Ring Z is a ring selected from phenyl, a 4-7 membered saturated or partially unsaturated carbocyclic ring, a 4-7 membered saturated or partially unsaturated heterocyclic ring with 1-3 heteroatoms independently selected from nitrogen, oxygen, and sulfur, and a 5-6 membered heteroaromatic ring with 1-4 heteroatoms independently selected from nitrogen, oxygen and sulfur;
Ring Y is a ring selected from phenylenyl, a 4-7 membered saturated or partially unsaturated carbocyclylenyl, a 4-7 membered saturated or partially unsaturated heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
each of L^{B} and L^{1′} is independently a covalent bond or a covalent bond or a bivalent, saturated or partially unsaturated, straight or branched Cis hydrocarbon chain, wherein 0-3 methylene units of L^{B} or L^{1′} are independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, or —OC(O)NR—, —NRC(O)O—;
each R^{y}, and R^{z} is independently selected from hydrogen, R^{B}, halogen, CN, —NO₂, —OR, —SR, —NR₂, —S(O)₂R, —S(O)₂NR₂, —S(O)R, —C(O)R, —C(O)OR, —C(O)NR₂, —C(O)NROR, —OC(O)R, —OC(O)NR₂, —NRC(O)OR, —NRC(O)R, —NRC(O)N(R)₂, and —NRS(O)₂R;
each R^{B} is independently an optionally substituted group selected from C₁₋₆ aliphatic, phenyl, a 3-12 membered saturated or partially unsaturated monocyclic, bicyclic, bridged bicyclic or spirocyclic carbocyclyl or heterocyclyl with 1-3 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or;
   two R groups on the same nitrogen are optionally taken together with their intervening atoms to form a 4-7 membered saturated, partially unsaturated, or heteroaryl ring having 0-3 heteroatoms, in addition to the nitrogen atom to which they are attached, independently selected from nitrogen, oxygen, and sulfur; and
y and z are independently 0, 1, 2, 3, or 4.

In some embodiments, the present invention provides a compound of formula I-gg, wherein DBM is a deubiquitinase binding moiety thereby forming a compound of formula I-gg-1:

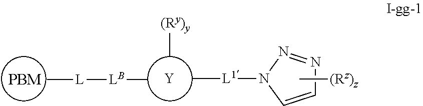

or a pharmaceutically acceptable salt thereof, wherein each of ring Y, L^{B}, L^{1′}, R^{y}, R^{z}, y, z, L, and PBM is as defined above and described in embodiments herein, both singly and in combination.

In some embodiments, the present invention provides a compound of formula I-gg, wherein DBM is a deubiquitinase binding moiety thereby forming a compound of formula I-gg-2:

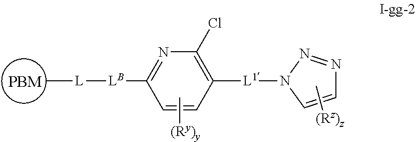

or a pharmaceutically acceptable salt thereof, wherein each of L^{B}, L^{1′}, R^{y}, R^{z}, y, z, L, and PBM is as defined above and described in embodiments herein, both singly and in combination.

In some embodiments, the present invention provides a compound of formula I-gg, wherein DBM is a deubiquitinase binding moiety thereby forming a compound of formula I-gg-3:

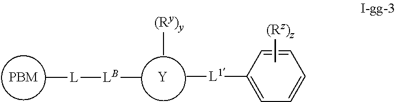

or a pharmaceutically acceptable salt thereof, wherein each of ring Y, L^{B}, L^{1′}, R^{y}, R^{z}, y, z, L, and PBM is as defined above and described in embodiments herein, both singly and in combination.

In some embodiments, the present invention provides a compound of formula I-gg, wherein DBM is a deubiquitinase binding moiety thereby forming a compound of formula I-gg-4:

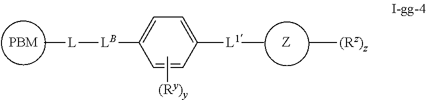

or a pharmaceutically acceptable salt thereof, wherein each of ring Z, L^{B}, L^{1′}, R^{y}, R^{z}, y, z, L, and PBM is as defined above and described in embodiments herein, both singly and in combination.

In some embodiments, the present invention provides a compound of formula I-gg, wherein DBM is a deubiquitinase binding moiety thereby forming a compound of formula I-gg-5:

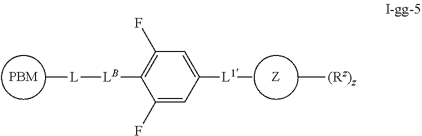

or a pharmaceutically acceptable salt thereof, wherein each of ring Z, L^{B}, L^{1′}, R^{z}, z, L, and PBM is as defined above and described in embodiments herein, both singly and in combination.

In some embodiments, the present invention provides a compound of formula I-aa, wherein DBM is a deubiquitinase binding moiety thereby forming a compound of formula I-hh:

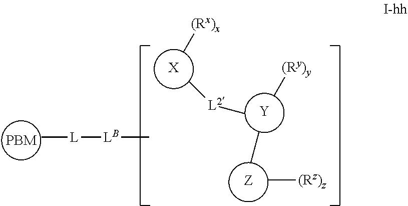

or a pharmaceutically acceptable salt thereof, wherein L and PBM are as defined above and described herein, and wherein:
Ring Z is a ring selected from phenyl, a 4-7 membered saturated or partially unsaturated carbocyclic ring, a 4-7 membered saturated or partially unsaturated heterocyclic ring with 1-3 heteroatoms independently selected from nitrogen, oxygen, and sulfur, a 5-6 membered heteroaromatic ring with 1-4 heteroatoms independently selected from nitrogen, oxygen and sulfur, and an 8-10 membered bicyclic heteroaromatic ring having 1-5 heteroatoms independently selected from nitrogen, oxygen, or sulfur; Ring Y is a ring selected from phenylenyl, a 4-7 membered saturated or partially unsaturated carbocyclylenyl, a 4-7 membered saturated or partially unsaturated heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
Ring X is a ring selected from phenylenyl, a 4-7 membered saturated or partially unsaturated carbocyclylenyl, a 4-7 membered saturated or partially unsaturated heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
each L^{B} and L¹ is independently a covalent bond or a bivalent, saturated or partially unsaturated, straight or branched C₁₋₅ hydrocarbon chain, wherein 0-3 methylene units of L^{B} or L^{2′} are independently replaced by —O—, —NR—, —S—, —OC(O)—, —C(O)O—, —C(O)—, —S(O)—, —S(O)—, —NRS(O)₂—, —S(O)₂NR—, —NRC(O)—, —C(O)NR—, or —OC(O)NR—, —NRC(O)O—;
each R^{x}, R^{y}, and R^{z} is independently selected from hydrogen, R^{B}, halogen, CN, —NO₂, —OR, —SR, —NR₂, —S(O)₂R, —S(O)₂NR₂, —S(O)R, —C(O)R, —C(O)OR, —C(O)NR₂, —C(O)NROR, —OC(O)R, —OC(O)NR₂, —NRC(O)OR, —NRC(O)R, —NRC(O)N(R)₂, and —NRS(O)₂R;
each R^{B} is independently an optionally substituted group selected from C₁₋₆ aliphatic, phenyl, a 3-12 membered saturated or partially unsaturated monocyclic, bicyclic, bridged bicyclic or spirocyclic carbocyclyl or heterocyclyl with 1-3 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or;
   two R groups on the same nitrogen are optionally taken together with their intervening atoms to form a 4-7 membered saturated, partially unsaturated, or heteroaryl ring having 0-3 heteroatoms, in addition to the nitrogen atom to which they are attached, independently selected from nitrogen, oxygen, and sulfur; and
x, y, and z are independently 0, 1, 2, 3, or 4.

In some embodiments, the present invention provides a compound of formula I-aa, wherein DBM is a deubiquitinase binding moiety thereby forming a compound of formula I-hh′:

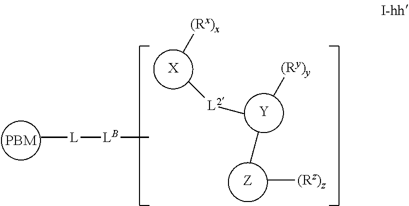

or a pharmaceutically acceptable salt thereof, wherein L and PBM are as defined above and described herein, and wherein:
Ring Z is a ring selected from phenyl, a 4-7 membered saturated or partially unsaturated carbocyclic ring, a 4-7 membered saturated or partially unsaturated heterocyclic ring with 1-3 heteroatoms independently selected from nitrogen, oxygen, and sulfur, a 5-6 membered heteroaromatic ring with 1-4 heteroatoms independently selected from nitrogen, oxygen and sulfur, and an 8-10 membered bicyclic heteroaromatic ring having 1-5 heteroatoms independently selected from nitrogen, oxygen, or sulfur;
Ring Y is a ring selected from phenylenyl, a 4-7 membered saturated or partially unsaturated carbocyclylenyl, a 4-7 membered saturated or partially unsaturated heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
Ring X is a ring selected from phenylenyl, a 4-7 membered saturated or partially unsaturated carbocyclylenyl, a 4-7 membered saturated or partially unsaturated heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
each L^{B} and L¹ is independently a covalent bond or a bivalent, saturated or partially unsaturated, straight or branched C₁₋₅ hydrocarbon chain, wherein 0-3 methylene units of L^{B} or L^{2′} are independently replaced by -Cy-, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, or —OC(O)NR—, —NRC(O)O—;
each R^{x}, R^{y}, and R^{z} is independently selected from hydrogen, R^{B}, halogen, CN, —NO₂, —OR, —SR, —NR₂, —S(O)₂R, —S(O)₂NR₂, —S(O)R, —C(O)R, —C(O)OR, —C(O)NR₂, —C(O)NROR, —OC(O)R. —OC(O)NR₂, —NRC(O)OR, —NRC(O)R, —NRC(O)N(R)₂, and —NRS(O)₂R;
each R^{B} is independently an optionally substituted group selected from C₁₋₆ aliphatic, phenyl, a 3-12 membered saturated or partially unsaturated monocyclic, bicyclic, bridged bicyclic or spirocyclic carbocyclyl or heterocyclyl with 1-3 heteroatoms independently selected from nitrogen, oxygen, and sulfur, and a 5-6 membered heteroaryl ring having 1-4 heteroatoms independently selected from nitrogen, oxygen, and sulfur;
each -Cy- is independently an optionally substituted bivalent ring selected from phenylenyl, an 8-10 membered bicyclic arylenyl, a 3-11 membered saturated or partially unsaturated monocyclic, bicyclic, bridged bicyclic or spirocyclic carbocyclylenyl, a 3-11 membered saturated or partially unsaturated monocyclic, bicyclic, bridged bicyclic or spirocyclic heterocyclylenyl having 1-3 heteroatoms independently selected from nitrogen, oxygen, and sulfur, a 5-6 membered heteroarylenyl having 1-4 heteroatoms independently selected from nitrogen, oxygen, and sulfur, and an 8-10 membered bicyclic heteroarylenyl having 1-5 heteroatoms independently selected from nitrogen, oxygen, and sulfur;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or;
   two R groups on the same nitrogen are optionally taken together with their intervening atoms to form a 4-7 membered saturated, partially unsaturated, or heteroaryl ring having 0-3 heteroatoms, in addition to the nitrogen atom to which they are attached, independently selected from nitrogen, oxygen, and sulfur; and
x, y, and z are independently 0, 1, 2, 3, or 4.

In some embodiments, the present invention provides a compound of formula I-hh or I-hh′, wherein DBM is a deubiquitinase binding moiety thereby forming a compound of formula I-hh-1:

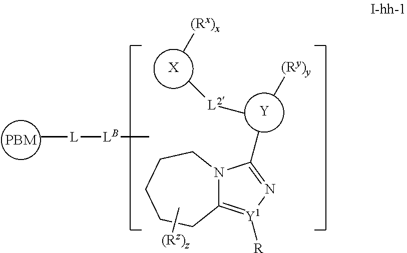

or a pharmaceutically acceptable salt thereof, wherein each of ring X, ring Y, L^{B}, L^{2′}, R^{x}, R^{y}, R^{z}, R, x, y, z, L, and PBM is as defined above and described in embodiments herein, both singly and in combination; wherein Y^{1′} is nitrogen or carbon.

In some embodiments, the present invention provides a compound of formula I-hh or I-hh′, wherein DBM is a deubiquitinase binding moiety thereby forming a compound of formula I-hh-2:

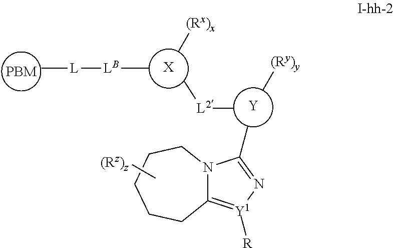

or a pharmaceutically acceptable salt thereof, wherein each of ring X, ring Y, L^{B}, L^{2′}, R^{x}, R^{y}, R^{z}, R, x, y, z, L, and PBM is as defined above and described in embodiments herein, both singly and in combination; wherein Y^{1′} is nitrogen or carbon.

In some embodiments, the present invention provides a compound of formula I-hh or I-hh′, wherein DBM is a deubiquitinase binding moiety thereby forming a compound of formula I-hh-3:

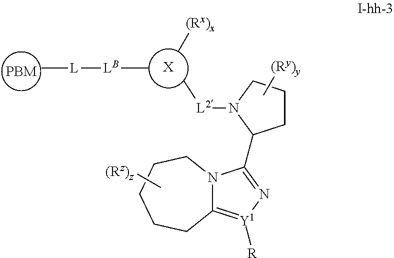

or a pharmaceutically acceptable salt thereof, wherein each of ring X, L^{B}, L^{2′}, R^{x}, R^{y}, R^{z}, R, x, y, z, L, and PBM is as defined above and described in embodiments herein, both singly and in combination; wherein Y¹ is nitrogen or carbon.

In some embodiments, the present invention provides a compound of formula I-hh or I-hh′, wherein DBM is a deubiquitinase binding moiety thereby forming a compound of formula I-hh-4 or I-hh′-4:

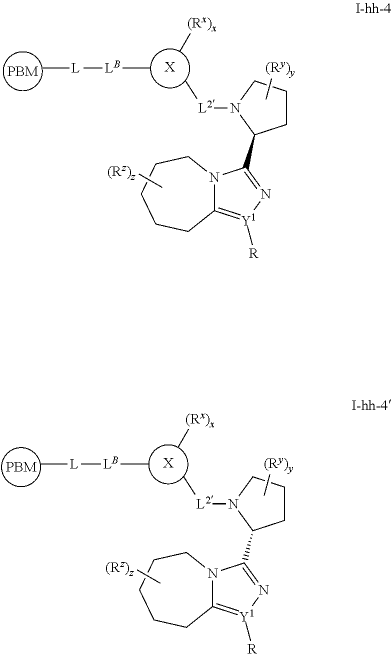

or a pharmaceutically acceptable salt thereof, wherein each of ring X, L^{B}, L^{2′}, R^{x}, R^{y}, R^{z}, R, x, y, z, L, and PBM is as defined above and described in embodiments herein, both singly and in combination; wherein Y′ is nitrogen or carbon.

In some embodiments, the present invention provides a compound of formula I-aa, wherein DBM is a deubiquitinase binding moiety thereby forming a compound of formula I-ii:

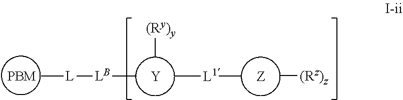

or a pharmaceutically acceptable salt thereof, wherein L and PBM are as defined above and described herein, and wherein:
Ring Z is a ring selected from phenyl, a 4-7 membered saturated or partially unsaturated carbocyclic ring, a 4-7 membered saturated or partially unsaturated heterocyclic ring with 1-3 heteroatoms independently selected from nitrogen, oxygen, and sulfur, and a 5-6 membered heteroaromatic ring with 1-4 heteroatoms independently selected from nitrogen, oxygen and sulfur;
Ring Y is a ring selected from phenylenyl, a 4-7 membered saturated or partially unsaturated carbocyclylenyl, a 4-7 membered saturated or partially unsaturated heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
each L^{B} and L¹ is a covalent bond or a bivalent, saturated or partially unsaturated, straight or branched C₁₋₅ hydrocarbon chain, wherein 0-3 methylene units of L^{B} or L^{1′} are independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, or —OC(O)NR—, —NRC(O)O—;
each R^{y}, and R^{z} is independently selected from hydrogen, R^{B}, halogen, CN, —NO₂, —OR, —SR, —NR₂, —S(O)₂R, —S(O)₂NR₂, —S(O)R, —C(O)R, —C(O)OR, —C(O)NR₂, —C(O)NROR, —OC(O)R, —OC(O)NR₂, —NRC(O)OR, —NRC(O)R, —NRC(O)N(R)₂, and —NRS(O)₂R;
each R^{B} is independently an optionally substituted group selected from C₁₋₆ aliphatic, phenyl, a 3-12 membered saturated or partially unsaturated monocyclic, bicyclic, bridged bicyclic or spirocyclic carbocyclyl or heterocyclyl with 1-3 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or;
   two R groups on the same nitrogen are optionally taken together with their intervening atoms to form a 4-7 membered saturated, partially unsaturated, or heteroaryl ring having 0-3 heteroatoms, in addition to the nitrogen atom to which they are attached, independently selected from nitrogen, oxygen, and sulfur; and
y, and z are independently 0, 1, 2, 3, or 4.

In some embodiments, the present invention provides a compound of formula I-ii, wherein DBM is a deubiquitinase binding moiety thereby forming a compound of formula I-ii-1:

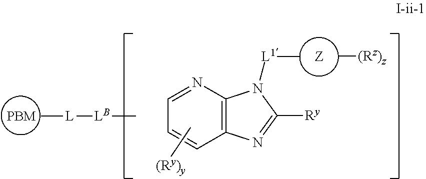

or a pharmaceutically acceptable salt thereof, wherein each of ring Z, L^{1′}, L^{B}, R^{y}, R^{z}, y, z, L, and PBM is as defined above and described in embodiments herein, both singly and in combination.

In some embodiments, the present invention provides a compound of formula I-ii, wherein DBM is a deubiquitinase binding moiety thereby forming a compound of formula I-ii-2:

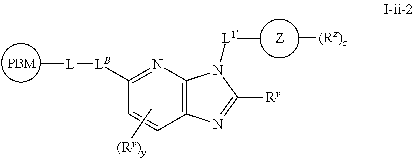

or a pharmaceutically acceptable salt thereof, wherein each of ring Z, L^{1′}, L^{B}, R^{y}, R^{z}, y, z, L, and PBM is as defined above and described in embodiments herein, both singly and in combination.

In some embodiments, the present invention provides a compound of formula I-ii, wherein DBM is a deubiquitinase binding moiety thereby forming a compound of formula I-jj-1:

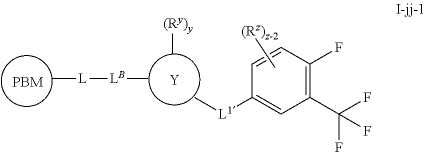

or a pharmaceutically acceptable salt thereof, wherein each of ring Y, L^{1′}, L^{B}, R^{y}, R^{z}, y, z, L, and PBM is as defined above and described in embodiments herein, both singly and in combination.

In some embodiments, the present invention provides a compound of formula I-aa, wherein DBM is a deubiquitinase binding moiety thereby forming a compound of formula I-kk:

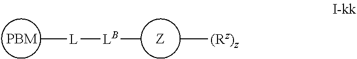

or a pharmaceutically acceptable salt thereof, wherein L and PBM are as defined above and described herein, and wherein:
Ring Z is a ring selected from phenyl, a 4-7 membered saturated or partially unsaturated carbocyclylenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, and an 8-10 membered bicyclic heteroaromatic ring having 1-5 heteroatoms independently selected from nitrogen, oxygen, or sulfur;
each R^{z} is independently selected from hydrogen, R^{B}, halogen, CN, —NO₂, —OR, —SR, —NR₂, —S(O)₂R, —S(O)₂NR₂, —S(O)R, —C(O)R, —C(O)OR, —C(O)NR₂, —C(O)NROR, —OC(O)R, —OC(O)NR₂, —NRC(O)OR, —NRC(O)R, —NRC(O)N(R)₂, and —NRS(O)₂R;
L^{B} is a covalent bond or a bivalent, saturated or partially unsaturated, straight or branched C₁₋₅ hydrocarbon chain, wherein 0-3 methylene units of L^{B} are independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, or —OC(O)NR—, —NRC(O)O—;
each R^{B} is independently an optionally substituted group selected from C₁₋₆ aliphatic, phenyl, a 3-12 membered saturated or partially unsaturated monocyclic, bicyclic, bridged bicyclic or spirocyclic carbocyclyl or heterocyclyl with 1-3 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or;
   two R groups on the same nitrogen are optionally taken together with their intervening atoms to form a 4-7 membered saturated, partially unsaturated, or heteroaryl ring having 0-3 heteroatoms, in addition to the nitrogen atom to which they are attached, independently selected from nitrogen, oxygen, and sulfur; and
z is 0, 1, 2, 3, or 4.

In some embodiments, the present invention provides a compound of formula I-aa, wherein DBM is a deubiquitinase binding moiety thereby forming a compound of formula I-kk-1:

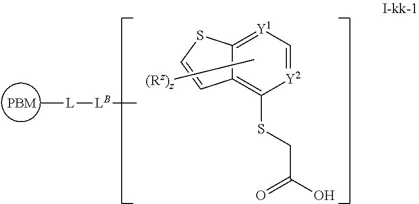

or a pharmaceutically acceptable salt thereof, wherein each of R^{z}, z, L^{B}, L, and PBM is as defined above and described in embodiments herein, both singly and in combination, wherein Y′ and Y² are independently carbon or nitrogen.

In some embodiments, the present invention provides a compound of formula I-kk, wherein DBM is a deubiquitinase binding moiety thereby forming a compound of formula I-kk-2:

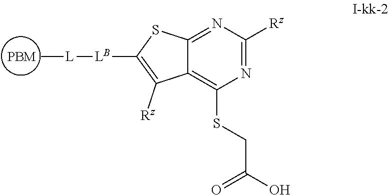

or a pharmaceutically acceptable salt thereof, wherein each of R^{z}, L^{B}, L, and PBM is as defined above and described in embodiments herein, both singly and in combination.

In some embodiments, the present invention provides a compound of formula I-aa, wherein DBM is a deubiquitinase binding moiety thereby forming a compound of formula I-II:

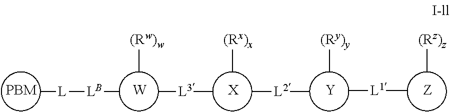

or a pharmaceutically acceptable salt thereof, wherein L and PBM are as defined above and described herein, and wherein:
Ring Z is a ring selected from phenyl, a 4-7 membered saturated or partially unsaturated carbocyclic ring, a 4-7 membered saturated or partially unsaturated heterocyclic ring with 1-3 heteroatoms independently selected from nitrogen, oxygen, and sulfur, and a 5-6 membered heteroaromatic ring with 1-4 heteroatoms independently selected from nitrogen, oxygen and sulfur;
Ring Y is a ring selected from phenylenyl, a 4-7 membered saturated or partially unsaturated carbocyclylenyl, a 4-7 membered saturated or partially unsaturated heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
Ring X is a ring selected from phenylenyl, a 4-7 membered saturated or partially unsaturated carbocyclylenyl, a 4-7 membered saturated or partially unsaturated heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
Ring W is a ring selected from phenylenyl, a 4-7 membered saturated or partially unsaturated carbocyclylenyl, a 4-7 membered saturated or partially unsaturated heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
each L^{B}, L^{1′}, and L^{3′} is independently a covalent bond or a bivalent, saturated or partially unsaturated, straight or branched C₁₋₅ hydrocarbon chain, wherein 0-3 methylene units of L^{B}, L^{1′}, or L^{3′} are independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, or —OC(O)NR—, —NRC(O)O—;
each R^{w}, R^{x}, R^{y}, and R^{z} is independently selected from hydrogen, R^{B}, halogen, —CN, —NO₂, —OR, —SR, —NR₂, —S(O)₂R, —S(O)₂NR₂, —S(O)R, —C(O)R, —C(O)OR, —C(O)NR₂, —C(O)NROR, —OC(O)R, —OC(O)NR₂, —NRC(O)OR, —NRC(O)R, —NRC(O)N(R)₂, and —NRS(O)₂R;
each R^{B} is independently an optionally substituted group selected from C₁₋₆ aliphatic, phenyl, a 3-12 membered saturated or partially unsaturated monocyclic, bicyclic, bridged bicyclic or spirocyclic carbocyclyl or heterocyclyl with 1-3 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or;
   two R groups on the same nitrogen are optionally taken together with their intervening atoms to form a 4-7 membered saturated, partially unsaturated, or heteroaryl ring having 0-3 heteroatoms, in addition to the nitrogen atom to which they are attached, independently selected from nitrogen, oxygen, and sulfur; and
w, x, y, and z are independently 0, 1, 2, 3, or 4.

In some embodiments, the present invention provides a compound of formula I, wherein DBM is a deubiquitinase binding moiety thereby forming a compound of formula I-II′:

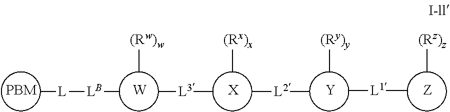

or a pharmaceutically acceptable salt thereof, wherein L and PBM are as defined above and described herein, and wherein:
Ring Z is a ring selected from phenylenyl, a 3-7 membered saturated or partially unsaturated monocyclic, bicyclic, bridged bicyclic or spirocyclic carbocyclylenyl, a 3-7 membered saturated or partially unsaturated monocyclic, bicyclic, bridged bicyclic or spirocyclic heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
Ring Y is a ring selected from phenylenyl, a 4-7 membered saturated or partially unsaturated carbocyclylenyl, a 4-7 membered saturated or partially unsaturated heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
Ring X is a ring selected from phenylenyl, a 4-7 membered saturated or partially unsaturated carbocyclylenyl, a 4-7 membered saturated or partially unsaturated heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
Ring W is a ring selected from phenylenyl, a 4-7 membered saturated or partially unsaturated carbocyclylenyl, a 4-7 membered saturated or partially unsaturated heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
each L^{B}, L^{1′}, and L^{3′} is independently a covalent bond or an optionally substituted bivalent, saturated or partially unsaturated, straight or branched C₁₋₅ hydrocarbon chain, wherein 0-3 methylene units of L^{B}, L^{1′}, or L^{3′} are independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, or —OC(O)NR—, —NRC(O)O—;
each R^{w}, R^{x}, R^{y}, and R^{z} is independently selected from hydrogen, R^{B}, halogen, —CN, —NO₂, —OR, —SR, —NR₂, —S(O)₂R, —S(O)₂NR₂, —S(O)R, —C(O)R, —C(O)OR, —C(O)NR₂, —C(O)NROR, —OC(O)R, —OC(O)NR₂, —NRC(O)OR, —NRC(O)R, —NRC(O)N(R)₂, and —NRS(O)₂R;
each R^{B} is independently an optionally substituted group selected from C₁₋₆ aliphatic, phenyl, a 3-12 membered saturated or partially unsaturated monocyclic, bicyclic, bridged bicyclic or spirocyclic carbocyclyl or heterocyclyl with 1-3 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or;
   two R groups on the same nitrogen are optionally taken together with their intervening atoms to form a 4-7 membered saturated, partially unsaturated, or heteroaryl ring having 0-3 heteroatoms, in addition to the nitrogen atom to which they are attached, independently selected from nitrogen, oxygen, and sulfur;
p is independently 0 or 1; and
w, x, y, and z are independently 0, 1, 2, 3, or 4.

In some embodiments, the present invention provides a compound of formula I-ll or I-ll′, wherein DBM is a deubiquitinase binding moiety thereby forming a compound of formula I-ll-1:

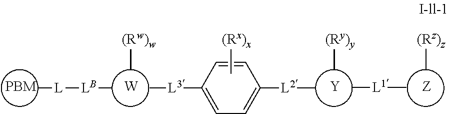

or a pharmaceutically acceptable salt thereof, wherein each of ring W, ring Y, ring Z, L^{B}, L^{3′}, L^{1′}, L^{1′}, R^{w}, R^{x}, R^{y}, R^{z}, w, x, y, z, L, and PBM is as defined above and described in embodiments herein, both singly and in combination.

In some embodiments, the present invention provides a compound of formula I-ll or I-ll′, wherein DBM is a deubiquitinase binding moiety thereby forming a compound of formula I-ll-2:

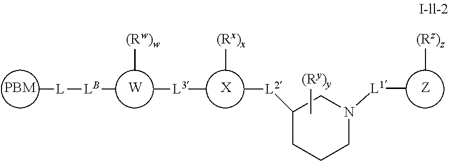

or a pharmaceutically acceptable salt thereof, wherein each of ring W, ring X, ring Z, L^{B}, L^{3′}, L^{2′}, L^{1′}, R^{w}, R^{x}, R^{y}, R^{z}, w, x, y, z, L, and PBM is as defined above and described in embodiments herein, both singly and in combination.

In some embodiments, the present invention provides a compound of formula I-ll or I-ll′, wherein DBM is a deubiquitinase binding moiety thereby forming a compound of formula I-ll-3:

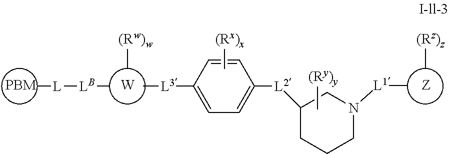

or a pharmaceutically acceptable salt thereof, wherein each of ring W, ring Z, L^{B}, L^{3′}, L^{2′}, L^{1′}, R^{w}, R^{x}, R^{y}, R^{z}, w, x, y, z, L, and PBM is as defined above and described in embodiments herein, both singly and in combination.

In some embodiments, the present invention provides a compound of formula I-ll or I-ll′, wherein DBM is a deubiquitinase binding moiety thereby forming a compound of formula I-ll-4:

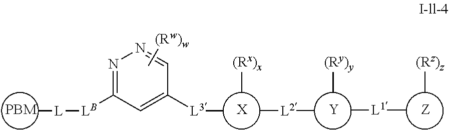

or a pharmaceutically acceptable salt thereof, wherein each of ring X, ring Y, ring Z, L^{B}, L^{3′}, L^{2′}, L^{1′}, R^{w}, R^{x}, R^{y}, R^{z}, w, x, y, z, L, and PBM is as defined above and described in embodiments herein, both singly and in combination.

In some embodiments, DBM is a covalent deubiquitinase binder. In some embodiments, DBM covalently binds OTUB1. In some embodiments, the DBM comprises a Michael accepter, e.g., α, β-unsaturated ketone which is capable of covalently binding to a deubiquitinase, e.g., OTUB1.

In some embodiments, the present invention provides a compound of formula I-aa, wherein DBM is a deubiquitinase binding moiety thereby forming a compound of formula I-mm:

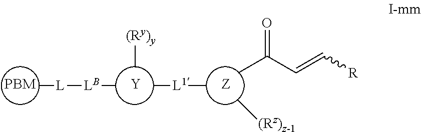

or a pharmaceutically acceptable salt thereof, wherein L and PBM are as defined above and described herein, and wherein:
Ring Z is a ring selected from phenylenyl, a 4-7 membered saturated or partially unsaturated carbocyclylenyl, a 4-7 membered saturated or partially unsaturated heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
Ring Y is a ring selected from phenylenyl, a 4-7 membered saturated or partially unsaturated carbocyclylenyl, a 4-7 membered saturated or partially unsaturated heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
each of L^{B}, and L^{1′}, is independently a covalent bond or a bivalent, saturated or partially unsaturated, straight or branched C₁₋₅ hydrocarbon chain, wherein 0-3 methylene units of L^{B}, or L^{1′}, are independently replaced by —O—, —NR—, —S—, —OC(O)—, —C(O)O—, —C(O)—, —S(O)—, —S(O)₂—, —NRS(O)₂—, —S(O)—NR—, —NRC(O)—, —C(O)NR—, or —OC(O)NR—, —NRC(O)O—;
each R^{y}, and R^{z} is independently selected from hydrogen, R^{B}, halogen, CN, —NO₂, —OR, —SR, —NR₂, —S(O)₂R, —S(O)₂NR₂, —S(O)R, —C(O)R, —C(O)OR, —C(O)NR₂, —C(O)NROR, —OC(O)R, —OC(O)NR₂, —NRC(O)OR, —NRC(O)R, —NRC(O)N(R)₂, and —NRS(O)₂R;
each R^{B} is independently an optionally substituted group selected from C₁₋₆ aliphatic, phenyl, a 3-12 membered saturated or partially unsaturated monocyclic, bicyclic, bridged bicyclic or spirocyclic carbocyclyl or heterocyclyl with 1-3 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or;
   two R groups on the same nitrogen are optionally taken together with their intervening atoms to form a 4-7 membered saturated, partially unsaturated, or heteroaryl ring having 0-3 heteroatoms, in addition to the nitrogen atom to which they are attached, independently selected from nitrogen, oxygen, and sulfur; and y, and z are independently 0, 1, 2, 3, or 4.

In some embodiments, the present invention provides a compound of formula I-mm, wherein DBM is a deubiquitinase binding moiety thereby forming a compound of formula I-mm-1:

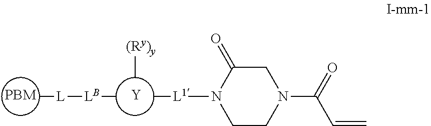

or a pharmaceutically acceptable salt thereof, wherein each of ring Y, L^{B}, L^{1′}, R^{y}, y, L, and PBM is as defined above and described in embodiments herein, both singly and in combination.

In some embodiments, the present invention provides a compound of formula I-mm, wherein DBM is a deubiquitinase binding moiety thereby forming a compound of formula I-mm-2:

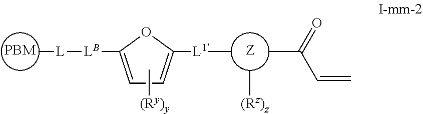

or a pharmaceutically acceptable salt thereof, wherein each of ring Z, L^{B}, L^{1′}, R^{y}, R^{z}, y, z, L, and PBM is as defined above and described in embodiments herein, both singly and in combination.

As defined above and described herein, ring Z is a ring selected from phenyl, a 4-7 membered saturated or partially unsaturated carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, and an 8-10 membered bicyclic heteroaromatic ring having 1-5 heteroatoms independently selected from nitrogen, oxygen, or sulfur.

As defined above and described herein, Ring Z is a ring selected from phenyl a 3-7 membered saturated or partially unsaturated monocyclic, bicyclic, bridged bicyclic or spirocyclic carbocyclic ring, a 3-7 membered saturated or partially unsaturated monocyclic, bicyclic, bridged bicyclic or spirocyclic heterocyclic ring with 1-3 heteroatoms independently selected from nitrogen, oxygen, and sulfur, and a 5-6 membered heteroaromatic ring with 1-4 heteroatoms independently selected from nitrogen, oxygen and sulfur.

In some embodiments, ring Z is a 4-7 membered saturated or partially unsaturated carbocyclylenyl. In some embodiments, ring Z is a phenyl. In some embodiments, ring Z is an 8-10 membered bicyclic aromatic carbocyclic ring. In some embodiments, ring Z is a 4-8 membered saturated or partially unsaturated monocyclic heterocyclic ring having 1-2 heteroatoms independently selected from nitrogen, oxygen, or sulfur. In some embodiments, ring Z is a 5-6 membered monocyclic heteroaromatic ring having 1-4 heteroatoms independently selected from nitrogen, oxygen, or sulfur. In some embodiments, ring Z is an 8-10 membered bicyclic heteroaromatic ring having 1-5 heteroatoms independently selected from nitrogen, oxygen, or sulfur.

In some embodiments, ring Z is a 3-7 membered saturated or partially unsaturated monocyclic, bicyclic, bridged bicyclic or spirocyclic carbocyclic ring. In some embodiments, ring Z is a 3-7 membered saturated or partially unsaturated monocyclic, bicyclic, bridged bicyclic or spirocyclic heterocyclic ring with 1-3 heteroatoms independently selected from nitrogen, oxygen, and sulfur. In some embodiments, ring Z is a 5-6 membered heteroaromatic ring with 1-4 heteroatoms independently selected from nitrogen, oxygen and sulfur.

In some embodiments, ring Z is cyclopropyl.

In some embodiments, ring Z is

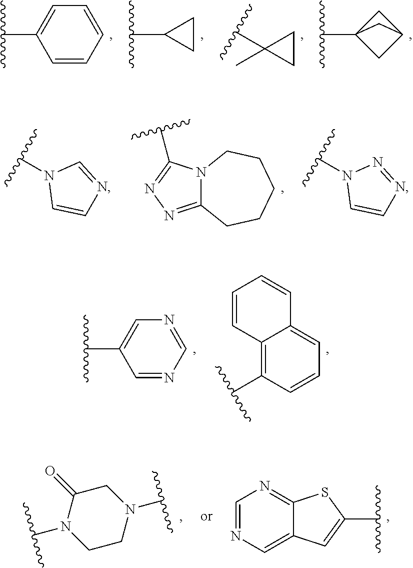

In some embodiments, ring Z is

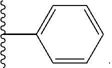 [SMILES: *c1ccccc1]

In some embodiments, ring Z is

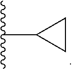 [SMILES: *C1CC1]

In some embodiments, ring Z is

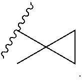 [SMILES: *C1(C)CC1]

In some embodiments, ring Z is

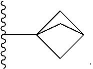 [SMILES: *C12CC(C1)C2]

In some embodiments, ring Z is

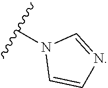 [SMILES: *n1ccnc1]

In some embodiments, ring Z is

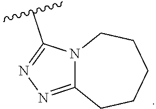 [SMILES: *c1nnc2n1CCCCC2]

In some embodiments, ring Z is

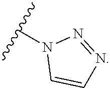 [SMILES: *n1ccnn1]

In some embodiments, ring Z is

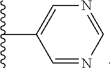 [SMILES: *c1cncnc1]

In some embodiments, ring Z is

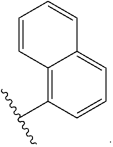 [SMILES: *c1cccc2ccccc12]

In some embodiments, ring Z is

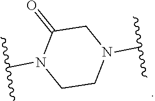 [SMILES: *N1CCN(*)C(=O)C1]

In some embodiments, ring Z is

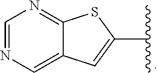 [SMILES: *c1cc2cncnc2s1]

In some embodiments, ring Z is selected from those depicted in the compounds of Table 1 below.

As defined above and described herein, ring Y is a ring selected from phenylenyl, 4-7 membered saturated or partially unsaturated carbocyclylenyl, an 8-10 membered bicyclic aromatic carbocyclylenyl, a 4-8 membered saturated or partially unsaturated monocyclic heterocyclylenyl having 1-2 heteroatoms independently selected from nitrogen, oxygen, or sulfur, a 5-6 membered monocyclic heteroarylenyl having 1-4 heteroatoms independently selected from nitrogen, oxygen, or sulfur, and an 8-10 membered bicyclic heteroarylenyl having 1-5 heteroatoms independently selected from nitrogen, oxygen, or sulfur.

In some embodiments, ring Y is a phenylenyl. In some embodiments, ring Y is a 4-7 membered saturated or partially unsaturated carbocyclylenyl. In some embodiments, ring Y is an 8-10 membered bicyclic aromatic carbocyclylenyl. In some embodiments, ring Y is a 4-8 membered saturated or partially unsaturated monocyclic heterocyclylenyl having 1-2 heteroatoms independently selected from nitrogen, oxygen, or sulfur. In some embodiments, ring Y is a 5-6 membered monocyclic heteroarylenyl having 1-4 heteroatoms independently selected from nitrogen, oxygen, or sulfur. In some embodiments, ring Y is an 8-10 membered bicyclic heteroarylenyl having 1-5 heteroatoms independently selected from nitrogen, oxygen, or sulfur.

In some embodiments, ring Y is

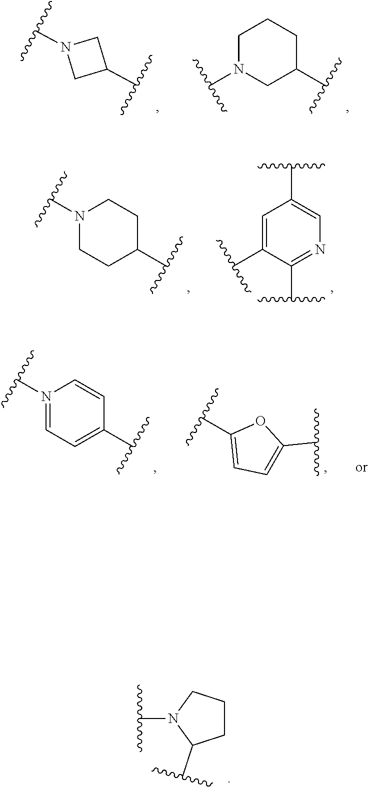

In some embodiments, ring Y is

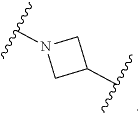 [SMILES: *C1CN(*)C1]

In some embodiments, ring Y is

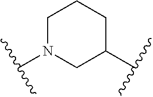 [SMILES: *C1CCCN(*)C1]

In some embodiments, ring Y is

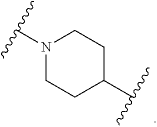 [SMILES: *C1CCN(*)CC1]

In some embodiments, ring Y is

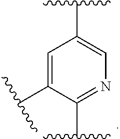 [SMILES: *c1cnc(*)c(*)c1]

In some embodiments, ring Y is

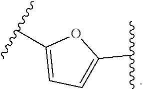 [SMILES: *c1ccc(*)o1]

In some embodiments, ring Y is

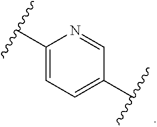 [SMILES: *c1ccc(*)nc1]

In some embodiments, ring Y is

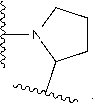 [SMILES: *C1CCCN1*]

In some embodiments, ring Y is selected from those depicted in the compounds of Table 1 below.

As defined above and described herein, ring X is a ring selected from phenylenyl, a 4-7 membered saturated or partially unsaturated carbocyclylenyl, a 4-7 membered saturated or partially unsaturated heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur.

In some embodiments, ring X is a phenylenyl. In some embodiments, ring X is a 4-7 membered saturated or partially unsaturated carbocyclylenyl. In some embodiments, ring X is a 4-7 membered saturated or partially unsaturated heterocyclylenyl with 1-3 heteroatoms independently selected from nitrogen, oxygen, and sulfur. In some embodiments, ring X is and a 5-6 membered heteroarylenyl with 1-4 heteroatoms independently selected from nitrogen, oxygen and sulfur.

In some embodiments, ring X is selected from those depicted in the compounds of Table 1 below.

As defined above and described herein, ring W is a ring selected from phenylenyl, a 4-7 membered saturated or partially unsaturated carbocyclylenyl, a 4-7 membered saturated or partially unsaturated heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur.

In some embodiments, ring W is a phenylenyl. In some embodiments, ring W is a 4-7 membered saturated or partially unsaturated carbocyclylenyl. In some embodiments, ring W is a 4-7 membered saturated or partially unsaturated heterocyclylenyl with 1-3 heteroatoms independently selected from nitrogen, oxygen, and sulfur. In some embodiments, ring W is and a 5-6 membered heteroarylenyl with 1-4 heteroatoms independently selected from nitrogen, oxygen and sulfur.

In some embodiments, ring W is cyclohexyl.

In some embodiments, ring W is selected from those depicted in the compounds of Table 1 below.

As defined above and described herein, L^{1′}, is a covalent bond or a bivalent, saturated or partially unsaturated, straight or branched C₁₋₅ hydrocarbon chain, wherein 0-3 methylene units of L^{1′}, L^{2′} or L^{B} are independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, or —OC(O)NR—, —NRC(O)O—.

In some embodiments, L^{1′} is a covalent bond. In some embodiments, L¹ is a bivalent, saturated or partially unsaturated, straight or branched C₁₋₅ hydrocarbon chain, wherein 0-3 methylene units of L^{1′} are independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, or —OC(O)NR—, —NRC(O)O—.

In some embodiments, L^{1′} is —O—. In some embodiments, L^{1′} is —NR—. In some embodiments, L¹ is —S—. In some embodiments, L^{1′} is —OC(O)—. In some embodiments, L^{1′} is —C(O)O—. In some embodiments, L^{1′} is —C(O)—. In some embodiments, L^{1′} is —S(O)—. In some embodiments, L^{1′} is —S(O)₂—. In some embodiments, L¹ is —NRS(O)₂—. In some embodiments, L^{1′} is —S(O)₂NR—. In some embodiments, L^{1′} is —NRC(O)—. In some embodiments, L^{1′} is —C(O)NR—. In some embodiments, L^{1′} is or —OC(O)NR—. In some embodiments, L¹ is —NRC(O)O—.

In some embodiments, L^{1′} is —CH₂—.

In some embodiments, L¹ is

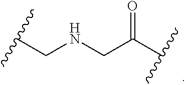 [SMILES: *CNCC(*)=O]

In some embodiments, L^{1′} is

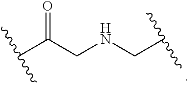 [SMILES: *CNCC(*)=O]

In some embodiments, L¹ is

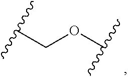 [SMILES: *CO*]

In some embodiments, L¹ is

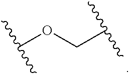 [SMILES: *CO*]

In some embodiments, L^{1′} is

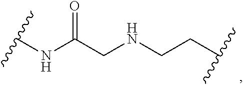 [SMILES: *CCNCC(=O)N*]

In some embodiments, L^{1′} is

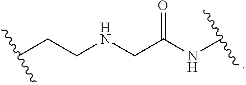 [SMILES: *CCNCC(=O)N* ; "."]

In some embodiments, L^{1′} is

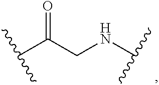 [SMILES: *NCC(*)=O]

In some embodiments, L^{1′} is selected from those depicted in the compounds of Table 1 below.

As defined above and described herein, L^{2′}, is a covalent bond or a bivalent, saturated or partially unsaturated, straight or branched C₁₋₅ hydrocarbon chain, wherein 0-3 methylene units of L^{2′} are independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, or —OC(O)NR—, —NRC(O)O—.

In some embodiments, L^{2′} is a covalent bond. In some embodiments, L^{2′} is a bivalent, saturated or partially unsaturated, straight or branched C₁₋₅ hydrocarbon chain, wherein 0-3 methylene units of L^{2′} are independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, or —OC(O)NR—, —NRC(O)O—.

In some embodiments, L^{2′} is —O—. In some embodiments, L^{2′} is —NR—. In some embodiments, L^{2′} is —S—. In some embodiments, L^{2′} is —OC(O)—. In some embodiments, L^{2′} is —C(O)O—. In some embodiments, L^{2′} is —C(O)—. In some embodiments, L^{2′} is —S(O)—. In some embodiments, L^{2′} is —S(O)₂—. In some embodiments, L^{2′} is —NRS(O)₂—. In some embodiments, L^{2′} is —S(O)₂NR—. In some embodiments, L^{2′} is —NRC(O)—. In some embodiments, L^{2′} is —C(O)NR—. In some embodiments, L^{2′} is or —OC(O)NR—. In some embodiments, L^{2′} is —NRC(O)O—. In some embodiments, L^{2′} is

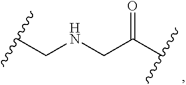 [SMILES: *CNCC(*)=O]

In some embodiments, L^{2′} is

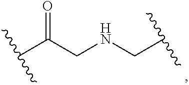 [SMILES: *CNCC(*)=O]

In some embodiments, L^{2′} is

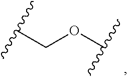 [SMILES: *CO*]

In some embodiments, L^{2′} is

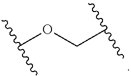 [SMILES: *CO*]

In some embodiments, L^{2′} is

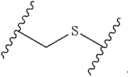 [SMILES: *CS*]

In some embodiments, L^{2′} is

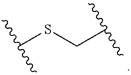 [SMILES: *CS*]

In some embodiments, L^{2′}, is —CH₂—. In some embodiments, L^{2′} is

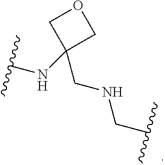 [SMILES: *CNCC1(N*)COC1]

In some embodiments, L^{2′} is

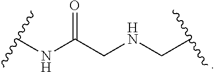 [SMILES: *CNCC(=O)N* ; "."]

In some embodiments, L^{2′} is

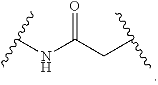 [SMILES: *CC(=O)N*]

In some embodiments, L^{2′} is selected from those depicted in the compounds of Table 1 below.

As defined above and described herein, L^{3′} is a covalent bond or a bivalent, saturated or partially unsaturated, straight or branched C₁₋₅ hydrocarbon chain, wherein 0-3 methylene units of L^{3′} are independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, or —OC(O)NR—, —NRC(O)O—.

In some embodiments, L^{3′} is a covalent bond. In some embodiments, L^{3′} is a bivalent, saturated or partially unsaturated, straight or branched C₁₋₅ hydrocarbon chain, wherein 0-3 methylene units of L^{3′} are independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, or —OC(O)NR—, —NRC(O)O—.

In some embodiments, L^{3′} is —O—. In some embodiments, L^{3′} is —NR—. In some embodiments, L^{3′} is —S—. In some embodiments, L^{3′} is —OC(O)—. In some embodiments, L^{3′} is —C(O)O—. In some embodiments, L^{3′} is —C(O)—. In some embodiments, L^{3′} is —S(O)—. In some embodiments, L^{3′} is —S(O)₂—. In some embodiments, L^{3′} is —NRS(O)₃—. In some embodiments, L^{3′} is —S(O)₂NR—. In some embodiments, L^{3′} is —NRC(O)—. In some embodiments, L^{3′} is —C(O)NR—. In some embodiments, L^{3′} is or —OC(O)NR—. In some embodiments, L^{3′} is —NRC(O)O—.

In some embodiments, L^{3′} is selected from those depicted in the compounds of Table 1 below.

As defined above and described herein, L^{B}, is a covalent bond or a bivalent, saturated or partially unsaturated, straight or branched C₁₋₅ hydrocarbon chain, wherein 0-3 methylene units of L^{B} are independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, or —OC(O)NR—, —NRC(O)O—.

In some embodiments, L^{B} is a covalent bond. In some embodiments, L^{B} is a bivalent, saturated or partially unsaturated, straight or branched C₁₋₅ hydrocarbon chain, wherein 0-3 methylene units of L^{B} are independently replaced by —O—, —NR—, —S—, —OC(O)—, —C(O)O—, —C(O)—, —S(O)—, —S(O)—, —NRS(O)₂—, —S(O)₂NR—, —NRC(O)—, —C(O)NR—, or —OC(O)NR—, —NRC(O)O—.

In some embodiments, L^{B} is —O—. In some embodiments, L^{B} is —NR—. In some embodiments, L^{B} is —S—. In some embodiments, L^{B} is —OC(O)—. In some embodiments, L^{B} is —C(O)O—. In some embodiments, L^{B} is —C(O)—. In some embodiments, L^{B} is —S(O)—. In some embodiments, L^{B} is —S(O)₂—. In some embodiments, L^{B} is —NRS(O)₃—. In some embodiments, L^{B} is —S(O)₂NR—. In some embodiments, L^{B} is —NRC(O)—. In some embodiments, L^{B} is —C(O)NR—. In some embodiments, L^{B} is or —OC(O)NR—. In some embodiments, L^{B} is —NRC(O)O—.

In some embodiments, L^{B} is selected from those depicted in the compounds of Table 1 below.

As defined above and described herein, each R^{w} is independently selected from hydrogen, R^{B}, halogen, —CN, —NO₂, —OR, —SR, —NR₂, —S(O)₂R, —S(O)₂NR₂, —S(O)R, —C(O)R, —C(O)OR, —C(O)NR₂, —C(O)NROR, —OC(O)R, —OC(O)NR₂, —NRC(O)OR, —NRC(O)R, —NRC(O)N(R)₂, and —NRS(O)₂R.

In some embodiments, one or more of R^{w} is hydrogen. In some embodiments, one or more of R^{w} is R^{B}. In some embodiments, one or more of R^{w} is halogen. In some embodiments, one or more of R^{w} is —CN. In some embodiments, one or more of R^{w} is —NO₂. In some embodiments, one or more of R^{w} is —OR. In some embodiments, one or more of R^{w} is —SR. In some embodiments, one or more of R^{w} is —NR₂. In some embodiments, one or more of R^{w} is —S(O)₂R. In some embodiments, one or more of R^{w} is —S(O)₂NR₂. In some embodiments, one or more of R^{w} is —S(O)R. In some embodiments, one or more of R^{w} is —C(O)R. In some embodiments, one or more of R^{w} is —C(O)OR. In some embodiments, one or more of R^{w} is —C(O)NR₂. In some embodiments, one or more of R^{w} is —C(O)NROR. In some embodiments, one or more of R^{w} is —OC(O)R. In some embodiments, one or more of R^{w} is —OC(O)NR₂. In some embodiments, one or more of R^{w} is —NRC(O)OR. In some embodiments, one or more of R^{w} is —NRC(O)R. In some embodiments, one or more of R^{w} is —NRC(O)N(R)₂. In some embodiments, one or more of R^{w} is —NRS(O)₂R.

In some embodiments,

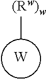

is

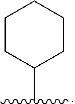 [SMILES: *C1CCCCC1 ; "."]

In some embodiments, R^{w} is selected from those depicted in the compounds of Table 1 below.

As defined above and described herein, each R^{x} is independently selected from hydrogen, R^{B}, halogen, —CN, —NO₂, —OR, —SR, —NR₂, —S(O)₂R, —S(O)₂NR₂, —S(O)R, —C(O)R, —C(O)OR, —C(O)NR₂, —C(O)NROR, —OC(O)R, —OC(O)NR₂, —NRC(O)OR, —NRC(O)R, —NRC(O)N(R)₂, and —NRS(O)₂R.

In some embodiments, one or more of R^{x} is hydrogen. In some embodiments, one or more of R^{x} is R^{B}. In some embodiments, one or more of R^{x} is halogen. In some embodiments, one or more of R^{x} is —CN. In some embodiments, one or more of R^{x} is —NO₂. In some embodiments, one or more of R^{x} is —OR. In some embodiments, one or more of R^{x} is —SR. In some embodiments, one or more of R^{x} is —NR₂. In some embodiments, one or more of R^{x} is —S(O)₂R. In some embodiments, one or more of R^{x} is —S(O)₂NR₂. In some embodiments, one or more of R^{x} is —S(O)R. In some embodiments, one or more of R$ is —C(O)R. In some embodiments, one or more of R^{x} is —C(O)OR. In some embodiments, one or more of R^{x} is —C(O)NR₂. In some embodiments, one or more of R^{x} is —C(O)NROR. In some embodiments, one or more of R^{x} is —OC(O)R. In some embodiments, one or more of R^{x} is —OC(O)NR₂. In some embodiments, one or more of R^{x} is —NRC(O)OR. In some embodiments, one or more of R is —NRC(O)R. In some embodiments, one or more of R^{x} is —NRC(O)N(R)₂. In some embodiments, one or more of R^{x} is —NRS(O)₂R.

In some embodiments,

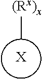

is

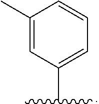 [SMILES: *c1cccc(C)c1 ; "."]

In some embodiments,

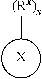

is

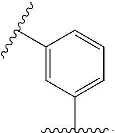 [SMILES: *c1cccc(*)c1 ; "."]

In some embodiments,

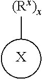

is

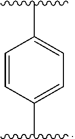 [SMILES: *c1ccc(*)cc1 ; "."]

In some embodiments,

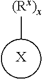

is

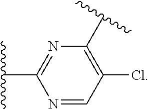 [SMILES: *c1ncc(Cl)c(*)n1]

In some embodiments,

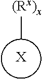

is

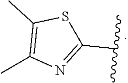 [SMILES: *c1nc(C)c(C)s1]

In some embodiments,

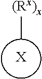

is

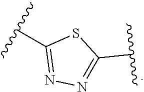 [SMILES: *c1nnc(*)s1]

In some embodiments,

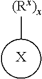

is

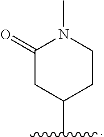 [SMILES: *C1CCN(C)C(=O)C1 ; "."]

In some embodiments,

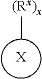

is

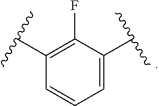 [SMILES: *c1cccc(*)c1F ; "."]

In some embodiments,

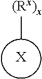

is

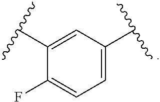 [SMILES: *c1ccc(F)c(*)c1 ; "."]

In some embodiments,

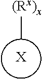

is

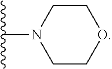 [SMILES: *N1CCOCC1]

In some embodiments,

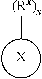

is

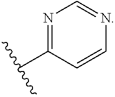 [SMILES: *c1ccncn1]

In some embodiments, R^{x} is selected from those depicted in the compounds of Table 1 below.

As defined above and described herein, each R^{y} is independently selected from hydrogen, R^{B}, halogen, —CN, —NO₂, —OR, —SR, —NR₂, —S(O)₂R, —S(O)₂NR₂, —S(O)R, —C(O)R, —C(O)OR, —C(O)NR₂, —C(O)NROR, —OC(O)R, —OC(O)NR₂, —NRC(O)OR, —NRC(O)R, —NRC(O)N(R)₂, and —NRS(O)₂R.

In some embodiments, one or more of R^{y} is hydrogen. In some embodiments, one or more of R^{y} is R^{B}. In some embodiments, one or more of R^{y} is halogen. In some embodiments, one or more of R^{y} is —CN. In some embodiments, one or more of R^{y} is —NO₂. In some embodiments, one or more of R^{y} is —OR. In some embodiments, one or more of R^{y} is —SR. In some embodiments, one or more of R^{y} is —NR₂. In some embodiments, one or more of R^{y} is —S(O)₂R. In some embodiments, one or more of R^{y} is —S(O)₂NR₂. In some embodiments, one or more of R^{y} is —S(O)R. In some embodiments, one or more of R^{y} is —C(O)R. In some embodiments, one or more of R^{y} is —C(O)OR. In some embodiments, one or more of R^{y} is —C(O)NR₂. In some embodiments, one or more of R^{y} is —C(O)NROR. In some embodiments, one or more of R^{y} is —OC(O)R. In some embodiments, one or more of R^{y} is —OC(O)NR₂. In some embodiments, one or more of R^{y} is —NRC(O)OR. In some embodiments, one or more of R^{y} is —NRC(O)R. In some embodiments, one or more of R^{y} is —NRC(O)N(R)₂. In some embodiments, one or more of R^{y} is —NRS(O)₂R.

In some embodiments, one or more of R^{y} is fluoro.

In some embodiments,

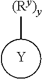

is

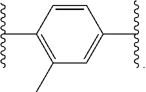 [SMILES: *c1ccc(*)c(C)c1 ; "."]

In some embodiments,

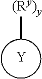

is

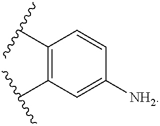 [SMILES: *c1ccc(N)cc1*]

In some embodiments,

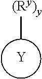

is

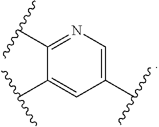 [SMILES: *c1cnc(*)c(*)c1]

In some embodiments,

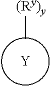

is

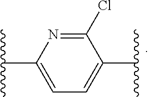 [SMILES: *c1ccc(*)c(Cl)n1]

In some embodiments,

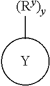

is

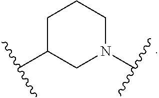 [SMILES: *C1CCCN(*)C1]

In some embodiments,

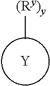

is

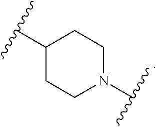 [SMILES: *C1CCN(*)CC1]

In some embodiments,

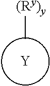

is

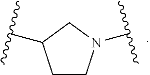 [SMILES: *C1CCN(*)C1]

In some embodiments,

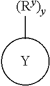

is

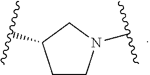 [SMILES: *[C@H]1CCN(*)C1]

In some embodiments,

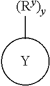

is

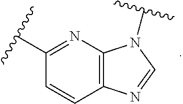 [SMILES: *c1ccc2ncn(*)c2n1]

In some embodiments, R^{y} is selected from those depicted in the compounds of Table 1 below.

As defined above and described herein, each R^{z} is independently selected from hydrogen, R^{B}, halogen, —CN, —NO₂, —OR, —SR, —NR₂, —S(O)₂R, —S(O)₂NR₂, —S(O)R, —C(O)R, —C(O)OR, —C(O)NR₂, —C(O)NROR, —OC(O)R, —OC(O)NR₂, —NRC(O)OR, —NRC(O)R, —NRC(O)N(R)₂, and —NRS(O)₂R.

In some embodiments, one or more of R^{z} is hydrogen. In some embodiments, one or more of R^{z} is R^{B}. In some embodiments, one or more of R^{z} is halogen. In some embodiments, one or more of R^{z} is —CN. In some embodiments, one or more of R^{z} is —NO₂. In some embodiments, one or more of R^{z} is —OR. In some embodiments, one or more of R^{z} is —SR. In some embodiments, one or more of R^{z} is —NR₂. In some embodiments, one or more of R^{z} is —S(O)R. In some embodiments, one or more of R^{z} is —S(O)₂NR₂. In some embodiments, one or more of R^{z} is —S(O)R. In some embodiments, one or more of R^{z} is —C(O)R. In some embodiments, one or more of R^{z} is —C(O)OR. In some embodiments, one or more of R^{z} is —C(O)NR₂. In some embodiments, one or more of R^{z} is —C(O)NROR. In some embodiments, one or more of R^{z} is —OC(O)R. In some embodiments, one or more of R^{z} is —OC(O)NR₂. In some embodiments, one or more of R^{z} is —NRC(O)OR. In some embodiments, one or more of R^{z} is —NRC(O)R. In some embodiments, one or more of R^{z} is —NRC(O)N(R)₂. In some embodiments, one or more of R^{z} is —NRS(O)₂R.

In some embodiments, one or more of R^{z} is methyl. In some embodiments, one or more of R^{z} is ethyl.

In some embodiments,

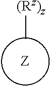

is

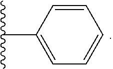 [SMILES: *c1ccccc1]

In some embodiments,

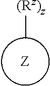

is

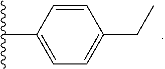 [SMILES: *c1ccc(CC)cc1]

In some embodiments,

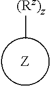

is

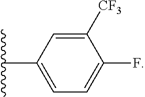 [SMILES: *c1ccc(F)c(C(F)(F)F)c1]

In some embodiments,

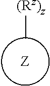

is

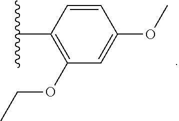 [SMILES: *c1ccc(OC)cc1OCC]

In some embodiments,

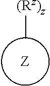

is

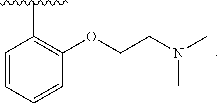 [SMILES: *c1ccccc1OCCN(C)C]

In some embodiments,

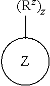

is

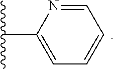 [SMILES: *c1ccccn1]

In some embodiments,

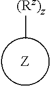

is

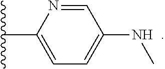 [SMILES: *c1ccc(NC)cn1]

In some embodiments,

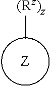

is

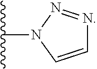 [SMILES: *n1ccnn1]

In some embodiments,

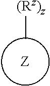

is

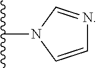 [SMILES: *n1ccnc1]

In some embodiments,

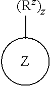

is

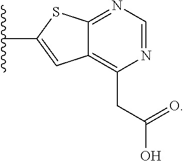 [SMILES: *c1cc2c(CC(=O)O)ncnc2s1]

In some embodiments,

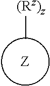

is

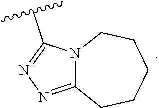 [SMILES: *c1nnc2n1CCCCC2]

In some embodiments,

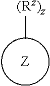

is

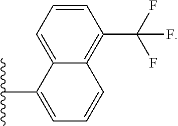 [SMILES: *c1cccc2c(C(F)(F)F)cccc12]

In some embodiments,

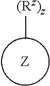

is

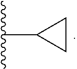 [SMILES: *C1CC1]

In some embodiments,

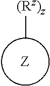

is

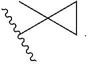 [SMILES: *C1(C)CC1]

In some embodiments,

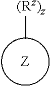

is

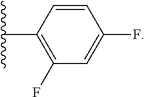 [SMILES: *c1ccc(F)cc1F]

In some embodiments, R^{z} is selected from those depicted in the compounds of Table 1 below.

As defined above and described herein, each R^{B} is independently an optionally substituted group selected from C₁₋₆ aliphatic, phenyl, a 3-12 membered saturated or partially unsaturated monocyclic, bicyclic, bridged bicyclic or spirocyclic carbocyclyl or heterocyclyl with 1-3 heteroatoms independently selected from nitrogen, oxygen, and sulfur, and a 5-6 membered heteroaryl ring having 1-4 heteroatoms independently selected from nitrogen, oxygen, and sulfur.

In some embodiments, of R^{B} is an optionally substituted C₁₋₆ aliphatic. In some embodiments, R^{B} is a phenyl. In some embodiments, R^{B} is a 3-12 membered saturated or partially unsaturated monocyclic. In some embodiments, R^{B} is an optionally substituted bicyclic. In some embodiments, of R^{B} is an optionally substituted bridged bicyclic or spirocyclic carbocyclyl. In some embodiments, R^{B} is a heterocyclyl with 1-3 heteroatoms independently selected from nitrogen, oxygen, and sulfur. In some embodiments, R^{B} is an optionally substituted 5-6 membered heteroaryl ring having 1-4 heteroatoms independently selected from nitrogen, oxygen, and sulfur.

In some embodiments, R^{B} is selected from those depicted in the compounds of Table 1 below.

As defined above and described herein, each -Cy′— is independently an optionally substituted bivalent ring selected from phenylenyl, an 8-10 membered bicyclic arylenyl, a 3-11 membered saturated or partially unsaturated monocyclic, bicyclic, bridged bicyclic or spirocyclic carbocyclylenyl, a 3-11 membered saturated or partially unsaturated monocyclic, bicyclic, bridged bicyclic or spirocyclic heterocyclylenyl having 1-3 heteroatoms independently selected from nitrogen, oxygen, and sulfur, a 5-6 membered heteroarylenyl having 1-4 heteroatoms independently selected from nitrogen, oxygen, and sulfur, and an 8-10 membered bicyclic heteroarylenyl having 1-5 heteroatoms independently selected from nitrogen, oxygen, and sulfur.

In some embodiments, one or more -Cy′- is an optionally substituted phenylenyl. In some embodiments, one or more -Cy′- is an optionally substituted 8-10 membered bicyclic arylenyl. In some embodiments, one or more -Cy′- is an optionally substituted 3-11 membered saturated or partially unsaturated monocyclic, bicyclic, bridged bicyclic or spirocyclic carbocyclylenyl. In some embodiments, one or more -Cy′- is an optionally substituted 3-11 membered saturated or partially unsaturated monocyclic, bicyclic, bridged bicyclic or spirocyclic heterocyclylenyl having 1-3 heteroatoms independently selected from nitrogen, oxygen, and sulfur. In some embodiments, one or more -Cy′- is an optionally substituted 5-6 membered heteroarylenyl having 1-4 heteroatoms independently selected from nitrogen, oxygen, and sulfur. In some embodiments, one or more -Cy′- is an optionally substituted 8-10 membered bicyclic heteroarylenyl having 1-5 heteroatoms independently selected from nitrogen, oxygen, and sulfur.

In some embodiments, -Cy′- is selected from those depicted in the compounds of Table 1 below.

As defined above and described herein, each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or two R groups on the same nitrogen are optionally taken together with their intervening atoms to form a 4-7 membered saturated, partially unsaturated, or heteroaryl ring having 0-3 heteroatoms, in addition to the nitrogen atom to which they are attached, independently selected from nitrogen, oxygen, and sulfur.

In some embodiments, one or more of R is hydrogen. In some embodiments, one or more of R is a C₁₋₆ aliphatic. In some embodiments, one or more of R is phenyl. In some embodiments, one or more of R is a 4-7 membered saturated or partially unsaturated heterocyclic having 1-2 heteroatoms independently selected from nitrogen, oxygen and sulfur. In some embodiments, one or more of R is and a 5-6 membered heteroaryl ring having 1-4 heteroatoms independently selected from nitrogen, oxygen, and sulfur. In some embodiments, two R groups on the same nitrogen are optionally taken together with their intervening atoms to form a 4-7 membered saturated, partially unsaturated, or heteroaryl ring having 0-3 heteroatoms, in addition to the nitrogen atom to which they are attached, independently selected from nitrogen, oxygen, and sulfur.

In some embodiments, R is selected from those depicted in the compounds of Table 1 below.

As defined above and described herein, m is independently 0 or 1. In some embodiments, m is 0. In some embodiments, m is 1.

In some embodiments, m is selected from those depicted in the compounds of Table 1 below.

As defined above and described herein, n is independently 0 or 1. In some embodiments, n is 0. In some embodiments, n is 1.

In some embodiments, n is selected from those depicted in the compounds of Table 1 below.

As defined above and described herein, o is independently 0 or 1. In some embodiments, o is 0. In some embodiments, o is 1.

In some embodiments, o is selected from those depicted in the compounds of Table 1 below.

As defined above and described herein, w is 0, 1, 2, 3, or 4. In some embodiments, w is 0. In some embodiments, w is 1. In some embodiments, w is 2. In some embodiments, w is 3. In some embodiments, w is 4.

In some embodiments, w is selected from those depicted in the compounds of Table 1 below.

As defined above and described herein, x is 0, 1, 2, 3, or 4. In some embodiments, x is 0. In some embodiments, x is 1. In some embodiments, x is 2. In some embodiments, x is 3. In some embodiments, x is 4.

In some embodiments, x is selected from those depicted in the compounds of Table 1 below.

As defined above and described herein, y is 0, 1, 2, 3, or 4. In some embodiments, y is 0. In some embodiments, y is 1. In some embodiments, y is 2. In some embodiments, y is 3. In some embodiments, y is 4.

In some embodiments, y is selected from those depicted in the compounds of Table 1 below.

As defined above and described herein, z is 0, 1, 2, 3, or 4. In some embodiments, z is 0. In some embodiments, z is 1. In some embodiments, z is 2. In some embodiments, z is 3. In some embodiments, z is 4.

In some embodiments, z is selected from those depicted in the compounds of Table 1 below.

In some embodiments, DBM is

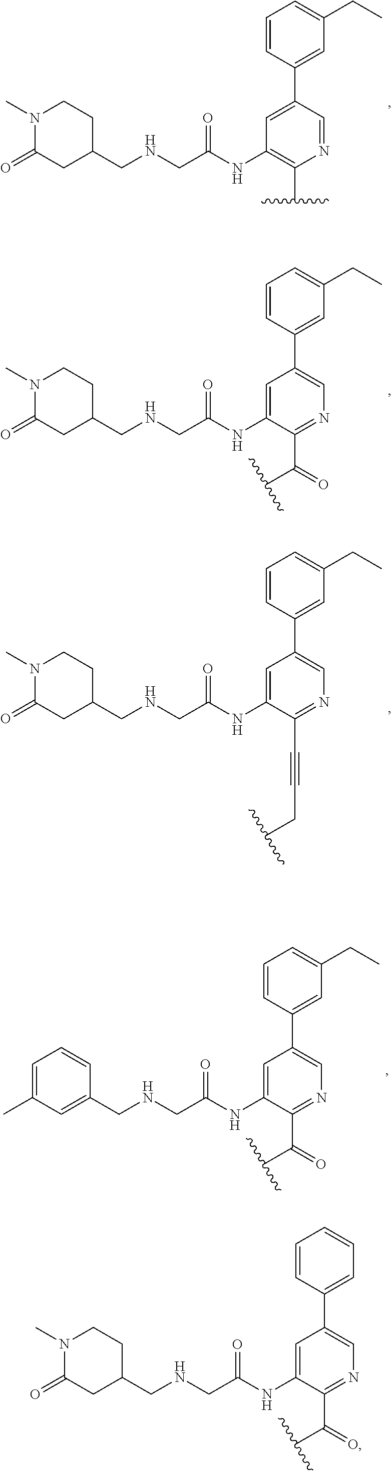

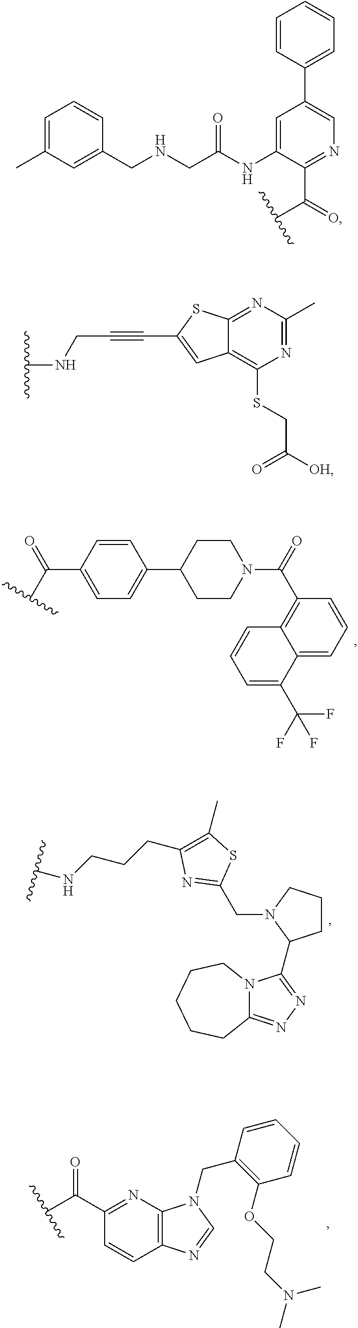 [SMILES: *C(=O)c1ncc(-c2ccccc2)cc1NC(=O)CNCc1cccc(C)c1 ; *NCC#Cc1cc2c(SCC(=O)O)nc(C)nc2s1 ; *C(=O)c1ccc(C2CCN(C(=O)c3cccc4c(C(F)(F)F)cccc34)CC2)cc1 ; *NCCCc1nc(CN2CCCC2c2nnc3n2CCCCC3)sc1C ; *C(=O)c1ccc2ncn(Cc3ccccc3OCCN(C)C)c2n1]

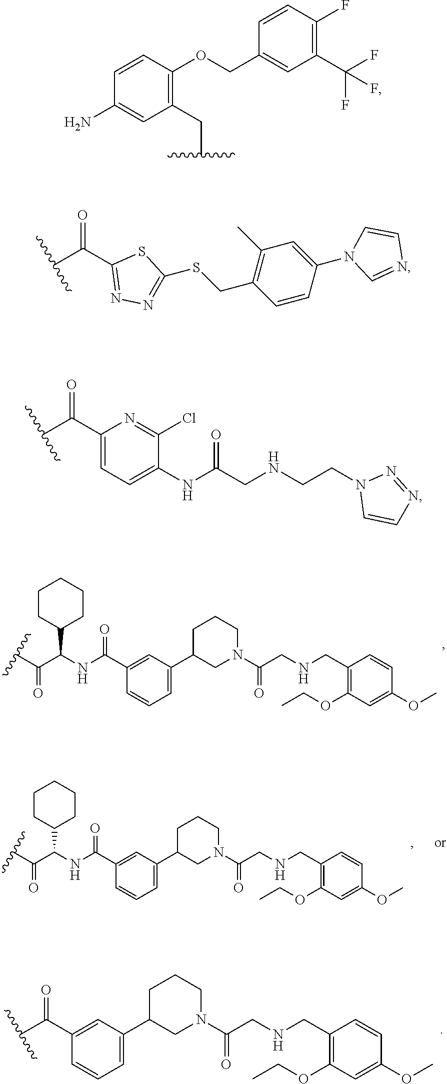 [SMILES: *Cc1cc(N)ccc1OCc1ccc(F)c(C(F)(F)F)c1 ; *C(=O)c1nnc(SCc2ccc(-n3ccnc3)cc2C)s1 ; *C(=O)c1ccc(NC(=O)CNCCn2ccnn2)c(Cl)n1 ; *C(=O)[C@H](NC(=O)c1cccc(C2CCCN(C(=O)CNCc3ccc(OC)cc3OCC)C2)c1)C1CCCCC1 ; *C(=O)[C@@H](NC(=O)c1cccc(C2CCCN(C(=O)CNCc3ccc(OC)cc3OCC)C2)c1)C1CCCCC1 ; *C(=O)c1cccc(C2CCCN(C(=O)CNCc3ccc(OC)cc3OCC)C2)c1]

In some embodiments, DBM is

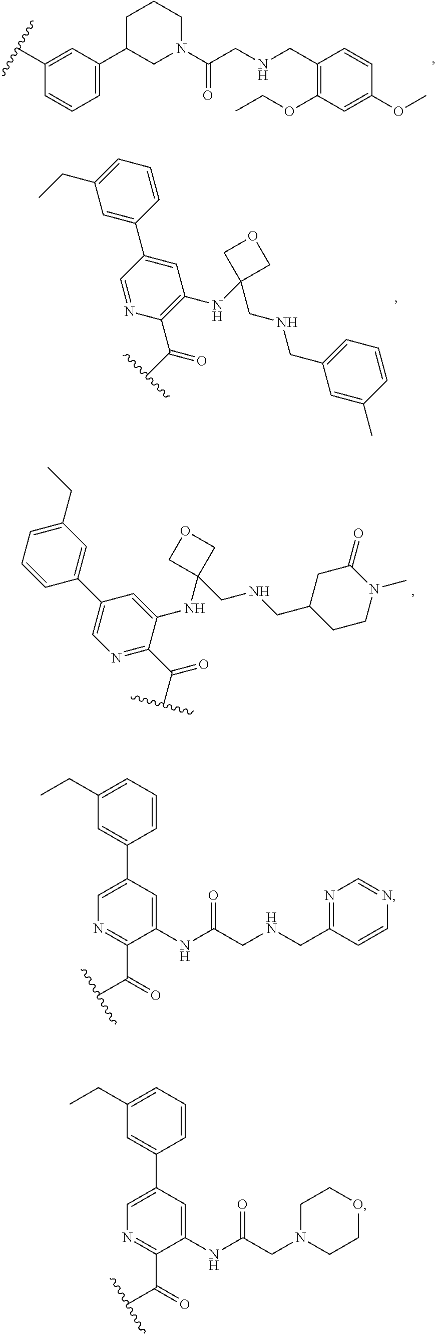

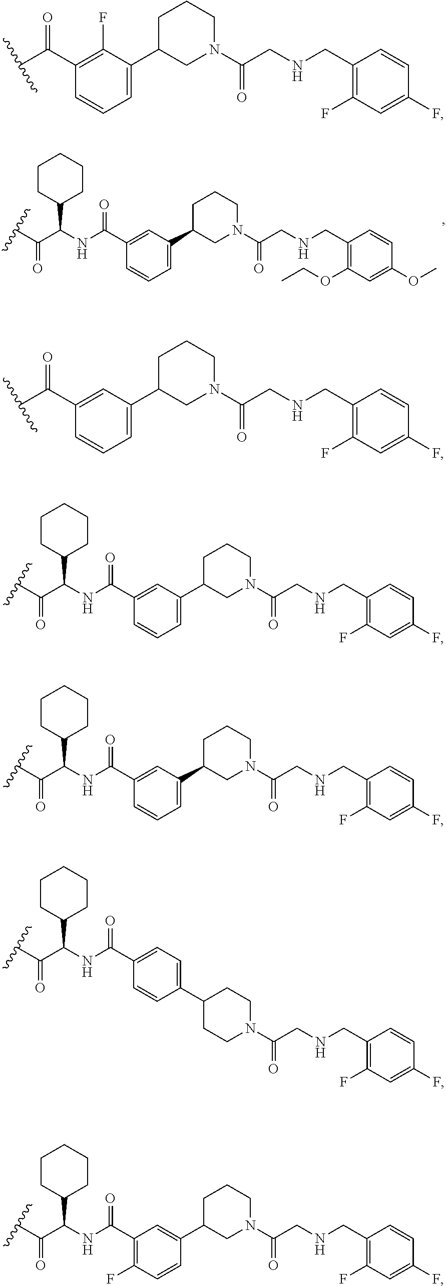

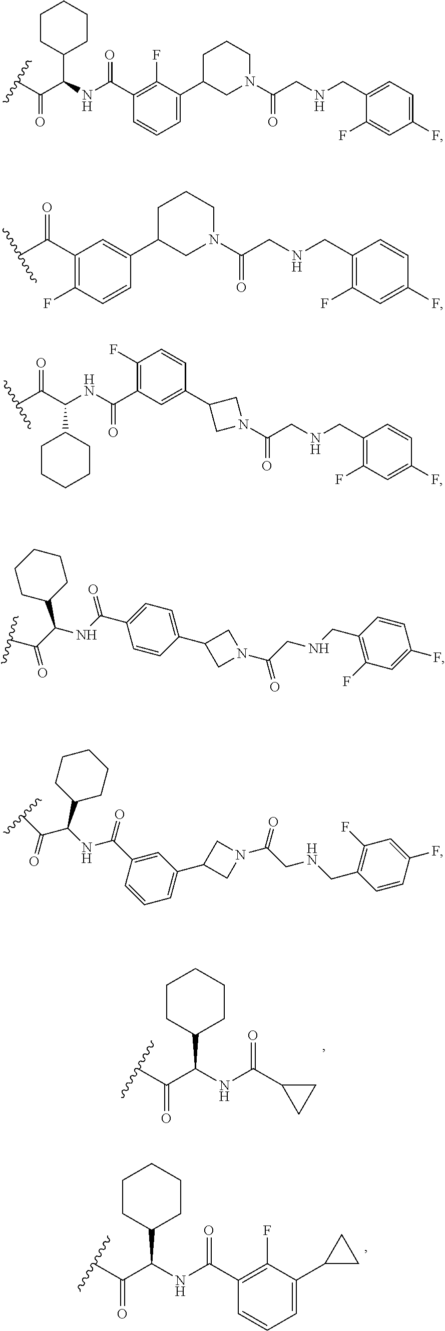

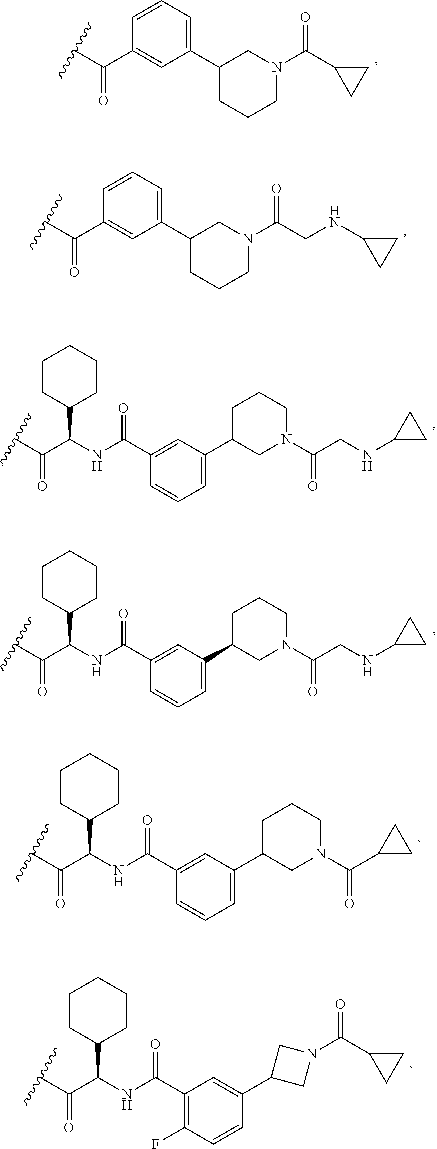 [SMILES: *C(=O)c1cccc(C2CCCN(C(=O)C3CC3)C2)c1 ; *C(=O)c1cccc(C2CCCN(C(=O)CNC3CC3)C2)c1 ; *C(=O)[C@H](NC(=O)c1cccc(C2CCCN(C(=O)CNC3CC3)C2)c1)C1CCCCC1 ; *C(=O)[C@H](NC(=O)c1cccc([C@H]2CCCN(C(=O)CNC3CC3)C2)c1)C1CCCCC1 ; *C(=O)[C@H](NC(=O)c1cccc(C2CCCN(C(=O)C3CC3)C2)c1)C1CCCCC1 ; *C(=O)[C@H](NC(=O)c1cc(C2CN(C(=O)C3CC3)C2)ccc1F)C1CCCCC1]

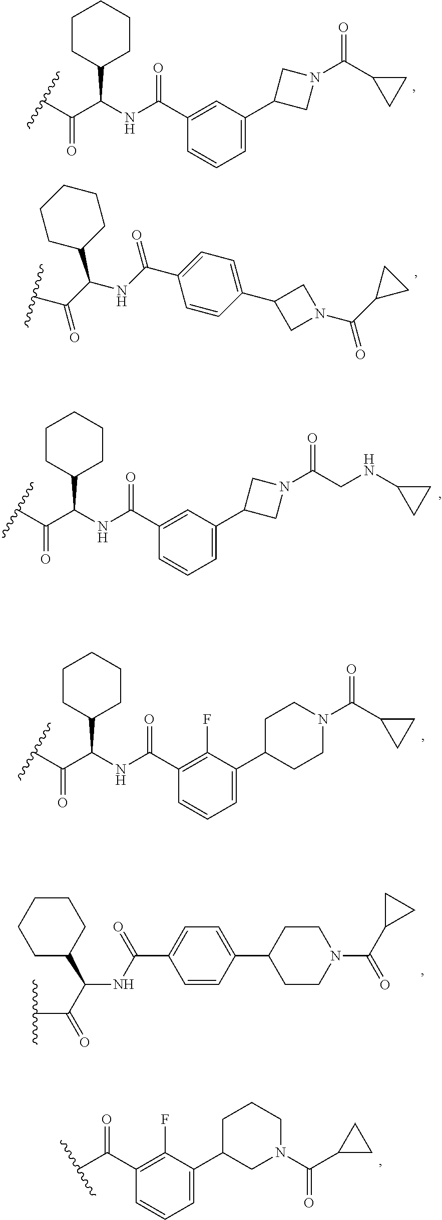

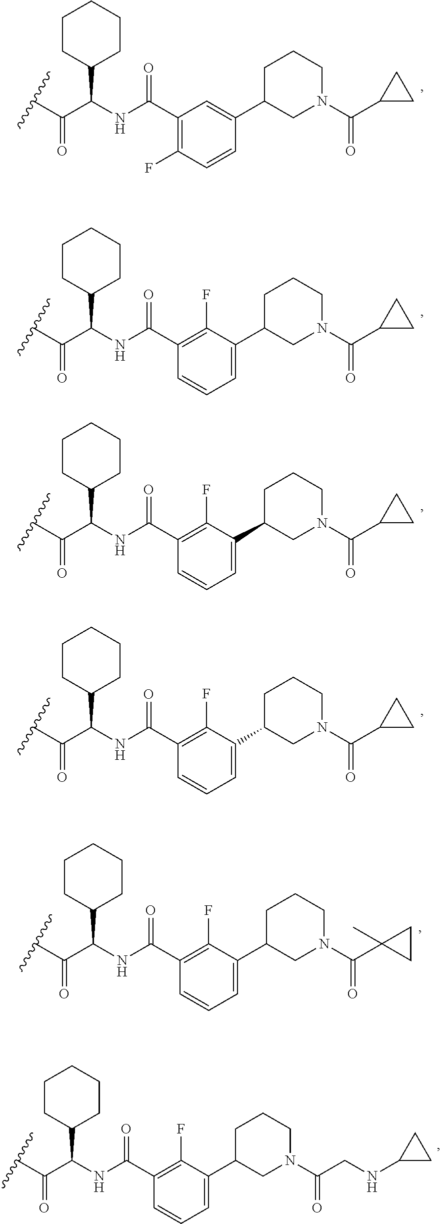 [SMILES: *C(=O)[C@H](NC(=O)c1cc(C2CCCN(C(=O)C3CC3)C2)ccc1F)C1CCCCC1 ; *C(=O)[C@H](NC(=O)c1cccc(C2CCCN(C(=O)C3CC3)C2)c1F)C1CCCCC1 ; *C(=O)[C@H](NC(=O)c1cccc([C@H]2CCCN(C(=O)C3CC3)C2)c1F)C1CCCCC1 ; *C(=O)[C@H](NC(=O)c1cccc([C@@H]2CCCN(C(=O)C3CC3)C2)c1F)C1CCCCC1 ; *C(=O)[C@H](NC(=O)c1cccc(C2CCCN(C(=O)C3(C)CC3)C2)c1F)C1CCCCC1 ; *C(=O)[C@H](NC(=O)c1cccc(C2CCCN(C(=O)CNC3CC3)C2)c1F)C1CCCCC1]

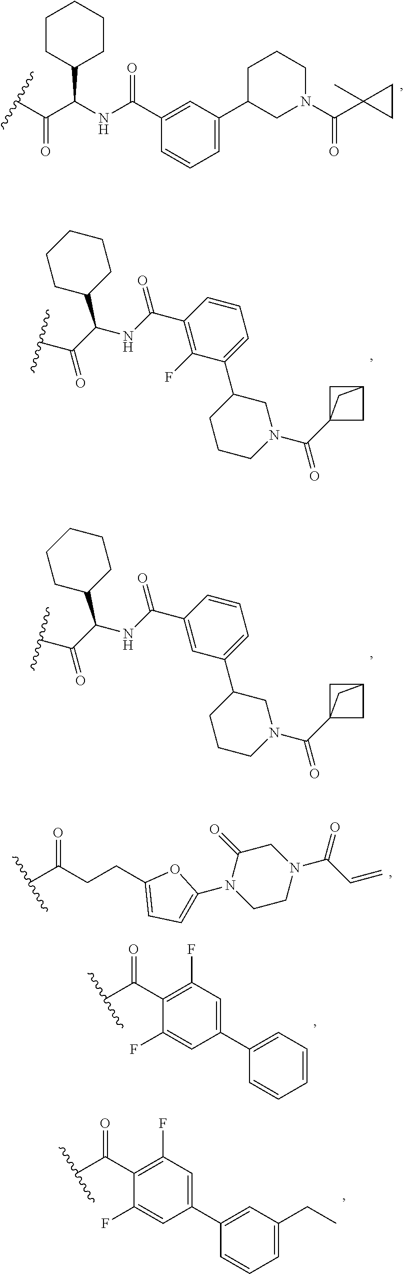

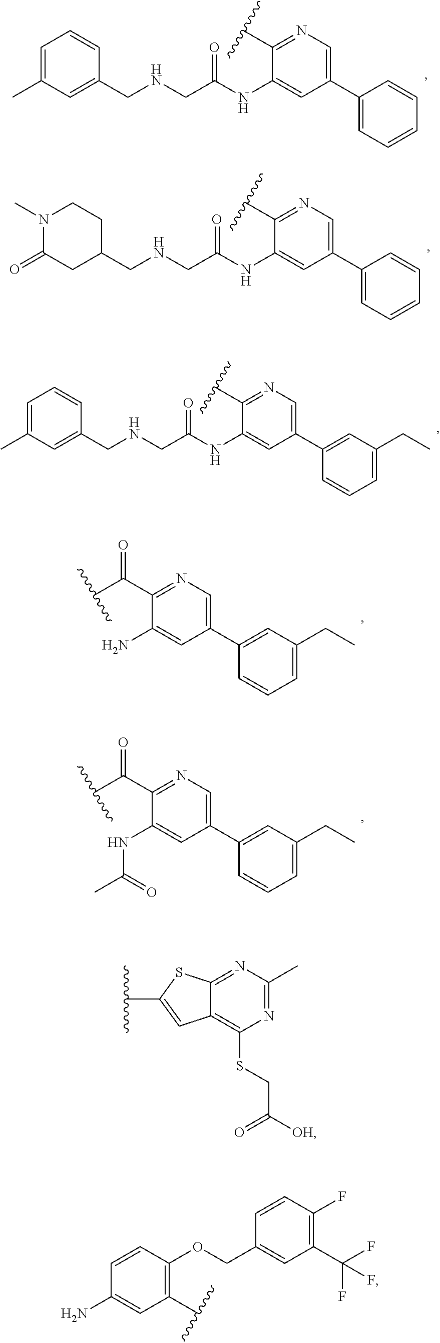

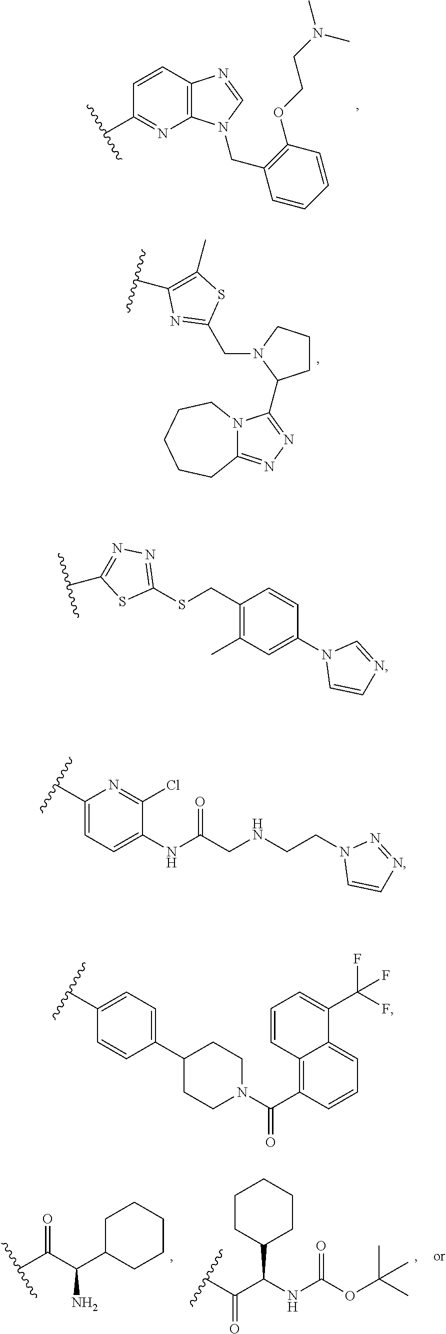

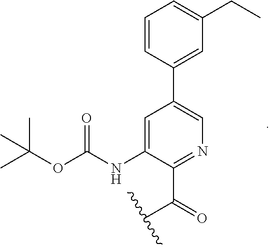 [SMILES: *C(=O)c1ncc(-c2cccc(CC)c2)cc1NC(=O)OC(C)(C)C]

In some embodiments, DBM is

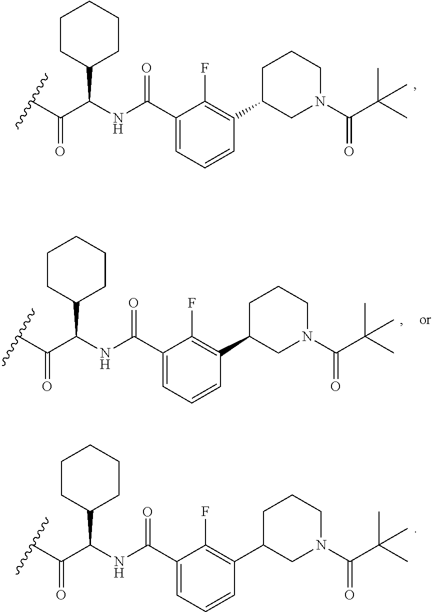 [SMILES: *C(=O)[C@H](NC(=O)c1cccc([C@@H]2CCCN(C(=O)C(C)(C)C)C2)c1F)C1CCCCC1 ; *C(=O)[C@H](NC(=O)c1cccc([C@H]2CCCN(C(=O)C(C)(C)C)C2)c1F)C1CCCCC1 ; *C(=O)[C@H](NC(=O)c1cccc(C2CCCN(C(=O)C(C)(C)C)C2)c1F)C1CCCCC1]

In some embodiments, DBM is

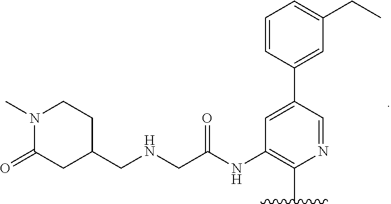 [SMILES: *c1ncc(-c2cccc(CC)c2)cc1NC(=O)CNCC1CCN(C)C(=O)C1]

In some embodiments, DBM is

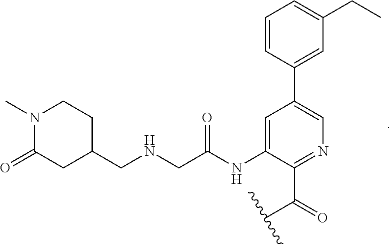 [SMILES: *C(=O)c1ncc(-c2cccc(CC)c2)cc1NC(=O)CNCC1CCN(C)C(=O)C1]

In some embodiments, DBM is

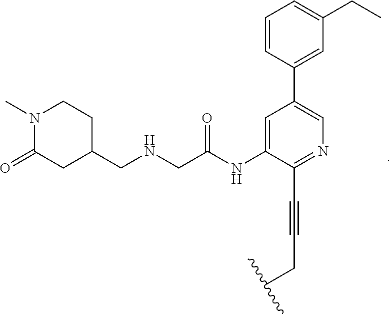 [SMILES: *CC#Cc1ncc(-c2cccc(CC)c2)cc1NC(=O)CNCC1CCN(C)C(=O)C1]

In some embodiments, DBM is

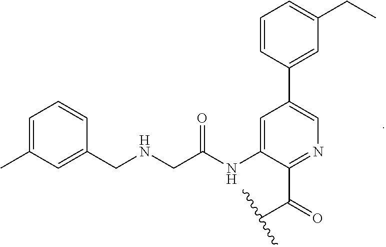 [SMILES: *C(=O)c1ncc(-c2cccc(CC)c2)cc1NC(=O)CNCc1cccc(C)c1]

In some embodiments, DBM is

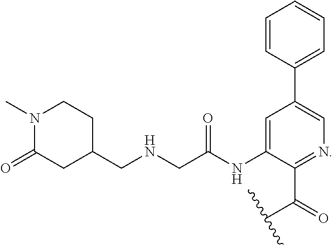 [SMILES: *C(=O)c1ncc(-c2ccccc2)cc1NC(=O)CNCC1CCN(C)C(=O)C1]

In some embodiments, DBM is

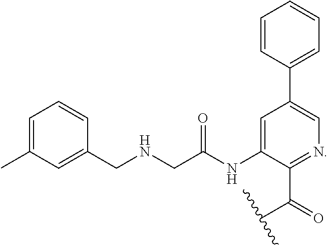 [SMILES: *C(=O)c1ncc(-c2ccccc2)cc1NC(=O)CNCc1cccc(C)c1]

In some embodiments, DBM is

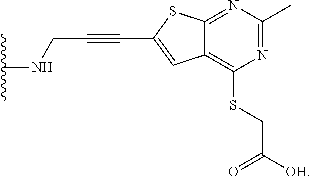 [SMILES: *NCC#Cc1cc2c(SCC(=O)O)nc(C)nc2s1]

In some embodiments, DBM is

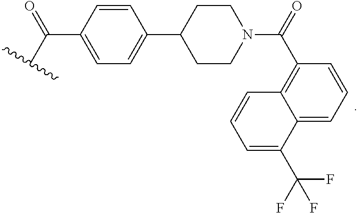 [SMILES: *C(=O)c1ccc(C2CCN(C(=O)c3cccc4c(C(F)(F)F)cccc34)CC2)cc1]

In some embodiments, DBM is

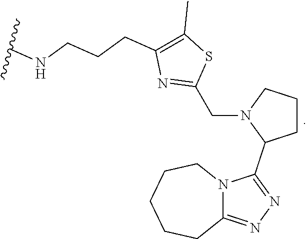 [SMILES: *NCCCc1nc(CN2CCCC2c2nnc3n2CCCCC3)sc1C]

In some embodiments, DBM is

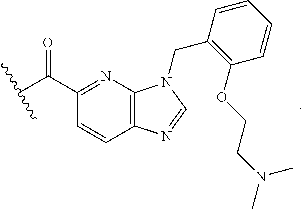 [SMILES: *C(=O)c1ccc2ncn(Cc3ccccc3OCCN(C)C)c2n1]

In some embodiments, DBM is

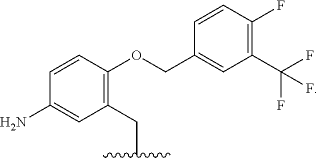 [SMILES: *Cc1cc(N)ccc1OCc1ccc(F)c(C(F)(F)F)c1]

In some embodiments, DBM is

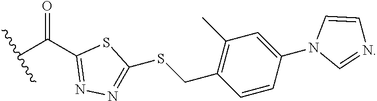 [SMILES: *C(=O)c1nnc(SCc2ccc(-n3ccnc3)cc2C)s1]

In some embodiments, DBM is

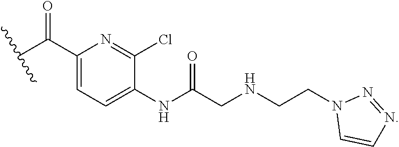 [SMILES: *C(=O)c1ccc(NC(=O)CNCCn2ccnn2)c(Cl)n1]

In some embodiments, DBM is

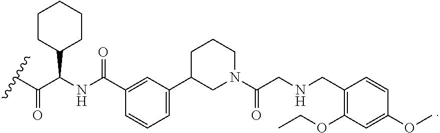 [SMILES: *C(=O)[C@H](NC(=O)c1cccc(C2CCCN(C(=O)CNCc3ccc(OC)cc3OCC)C2)c1)C1CCCCC1 ; "."]

In some embodiments, DBM is

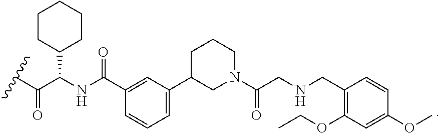 [SMILES: *C(=O)[C@@H](NC(=O)c1cccc(C2CCCN(C(=O)CNCc3ccc(OC)cc3OCC)C2)c1)C1CCCCC1 ; "."]

In some embodiments, DBM is

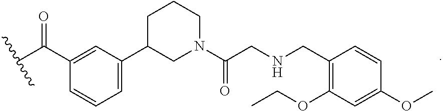 [SMILES: *C(=O)c1cccc(C2CCCN(C(=O)CNCc3ccc(OC)cc3OCC)C2)c1]

In some embodiments, DBM is

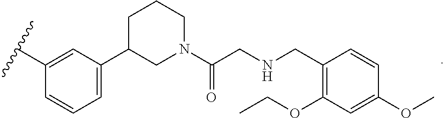 [SMILES: *c1cccc(C2CCCN(C(=O)CNCc3ccc(OC)cc3OCC)C2)c1]

In some embodiments, DBM is

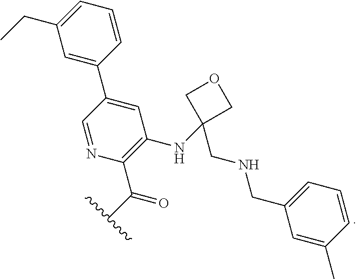 [SMILES: *C(=O)c1ncc(-c2cccc(CC)c2)cc1NC1(CNCc2cccc(C)c2)COC1 ; "."]

In some embodiments, DBM is

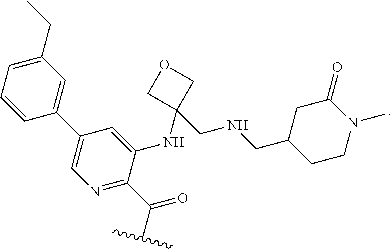 [SMILES: *C(=O)c1ncc(-c2cccc(CC)c2)cc1NC1(CNCC2CCN(C)C(=O)C2)COC1 ; "."]

In some embodiments, DBM is

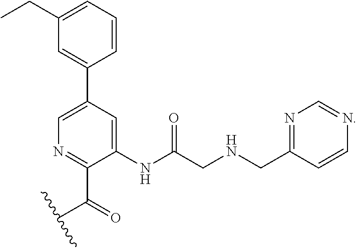 [SMILES: *C(=O)c1ncc(-c2cccc(CC)c2)cc1NC(=O)CNCc1ccncn1]

In some embodiments, DBM is

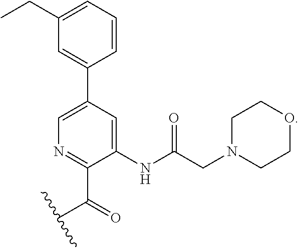 [SMILES: *C(=O)c1ncc(-c2cccc(CC)c2)cc1NC(=O)CN1CCOCC1]

In some embodiments, DBM is

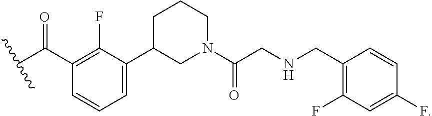 [SMILES: *C(=O)c1cccc(C2CCCN(C(=O)CNCc3ccc(F)cc3F)C2)c1F]

In some embodiments, DBM is

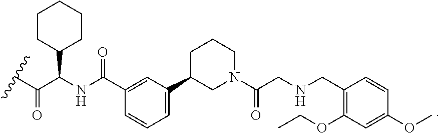 [SMILES: *C(=O)[C@H](NC(=O)c1cccc([C@H]2CCCN(C(=O)CNCc3ccc(OC)cc3OCC)C2)c1)C1CCCCC1 ; "."]

In some embodiments, DBM is

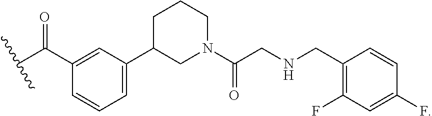 [SMILES: *C(=O)c1cccc(C2CCCN(C(=O)CNCc3ccc(F)cc3F)C2)c1]

In some embodiments, DBM is

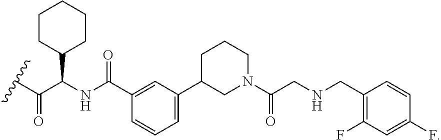 [SMILES: *C(=O)[C@H](NC(=O)c1cccc(C2CCCN(C(=O)CNCc3ccc(F)cc3F)C2)c1)C1CCCCC1]

In some embodiments, DBM is

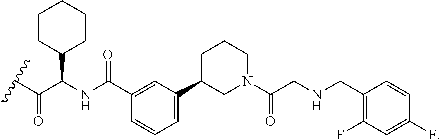 [SMILES: *C(=O)[C@H](NC(=O)c1cccc([C@H]2CCCN(C(=O)CNCc3ccc(F)cc3F)C2)c1)C1CCCCC1]

In some embodiments, DBM is

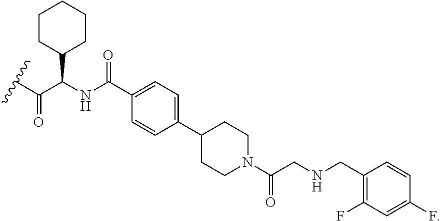 [SMILES: *C(=O)[C@H](NC(=O)c1ccc(C2CCN(C(=O)CNCc3ccc(F)cc3F)CC2)cc1)C1CCCCC1]

In some embodiments, DBM is

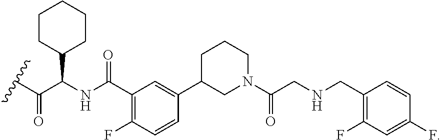 [SMILES: *C(=O)[C@H](NC(=O)c1cc(C2CCCN(C(=O)CNCc3ccc(F)cc3F)C2)ccc1F)C1CCCCC1]

In some embodiments, DBM is

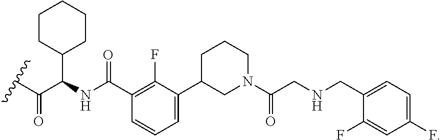 [SMILES: *C(=O)[C@H](NC(=O)c1cccc(C2CCCN(C(=O)CNCc3ccc(F)cc3F)C2)c1F)C1CCCCC1]

In some embodiments, DBM is

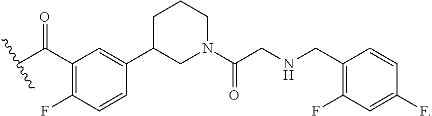 [SMILES: *C(=O)c1cc(C2CCCN(C(=O)CNCc3ccc(F)cc3F)C2)ccc1F]

In some embodiments, DBM is

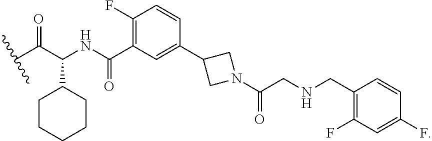 [SMILES: *C(=O)[C@H](NC(=O)c1cc(C2CN(C(=O)CNCc3ccc(F)cc3F)C2)ccc1F)C1CCCCC1]

In some embodiments, DBM is

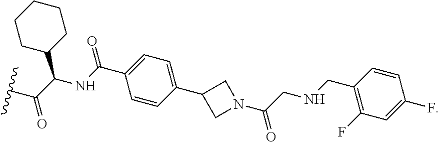 [SMILES: *C(=O)[C@H](NC(=O)c1ccc(C2CN(C(=O)CNCc3ccc(F)cc3F)C2)cc1)C1CCCCC1]

In some embodiments, DBM is

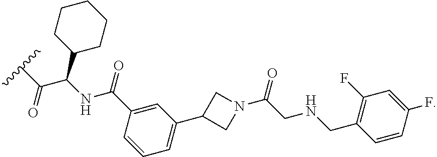 [SMILES: *C(=O)[C@H](NC(=O)c1cccc(C2CN(C(=O)CNCc3ccc(F)cc3F)C2)c1)C1CCCCC1]

In some embodiments, DBM is

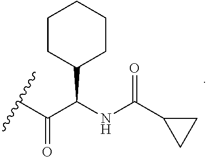 [SMILES: *C(=O)[C@H](NC(=O)C1CC1)C1CCCCC1]

In some embodiments, DBM is

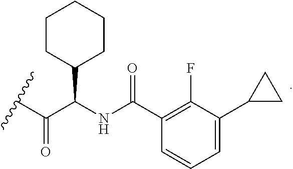 [SMILES: *C(=O)[C@H](NC(=O)c1cccc(C2CC2)c1F)C1CCCCC1]

In some embodiments, DBM is

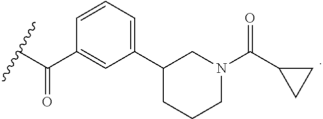 [SMILES: *C(=O)c1cccc(C2CCCN(C(=O)C3CC3)C2)c1]

In some embodiments, DBM is

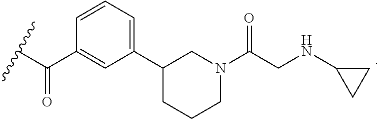 [SMILES: *C(=O)c1cccc(C2CCCN(C(=O)CNC3CC3)C2)c1]

In some embodiments, DBM is

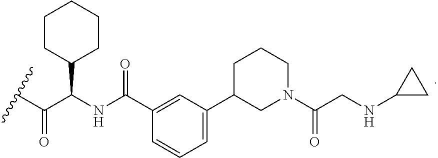 [SMILES: *C(=O)[C@H](NC(=O)c1cccc(C2CCCN(C(=O)CNC3CC3)C2)c1)C1CCCCC1]

In some embodiments, DBM
is

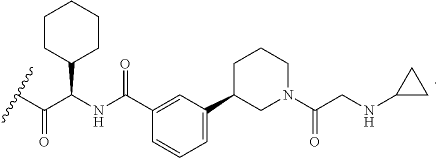 [SMILES: *C(=O)[C@H](NC(=O)c1cccc([C@H]2CCCN(C(=O)CNC3CC3)C2)c1)C1CCCCC1]

In some embodiments, DBM is

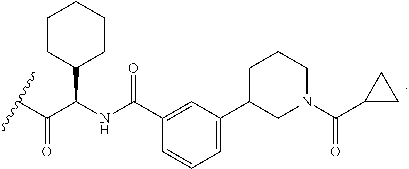 [SMILES: *C(=O)[C@H](NC(=O)c1cccc(C2CCCN(C(=O)C3CC3)C2)c1)C1CCCCC1]

In some embodiments, DBM is

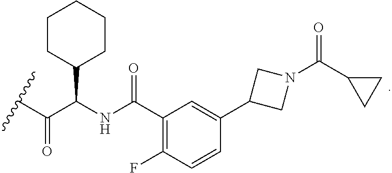 [SMILES: *C(=O)[C@H](NC(=O)c1cc(C2CN(C(=O)C3CC3)C2)ccc1F)C1CCCCC1]

In some embodiments, DBM is

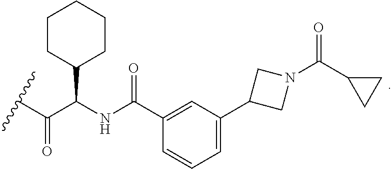 [SMILES: *C(=O)[C@H](NC(=O)c1cccc(C2CN(C(=O)C3CC3)C2)c1)C1CCCCC1]

In some embodiments, DBM is

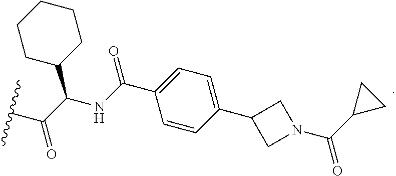 [SMILES: *C(=O)[C@H](NC(=O)c1ccc(C2CN(C(=O)C3CC3)C2)cc1)C1CCCCC1]

In some embodiments, DBM is

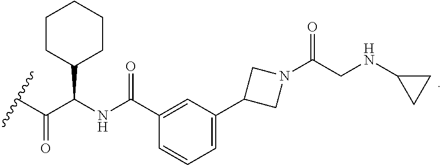 [SMILES: *C(=O)[C@H](NC(=O)c1cccc(C2CN(C(=O)CNC3CC3)C2)c1)C1CCCCC1]

In some embodiments, DBM is

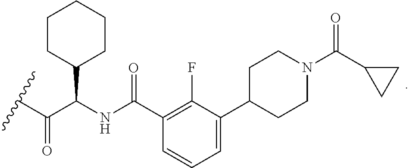 [SMILES: *C(=O)[C@H](NC(=O)c1cccc(C2CCN(C(=O)C3CC3)CC2)c1F)C1CCCCC1]

In some embodiments, DBM is

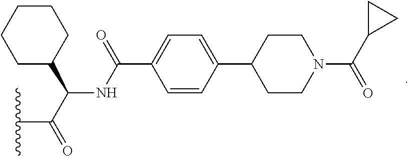 [SMILES: *C(=O)[C@H](NC(=O)c1ccc(C2CCN(C(=O)C3CC3)CC2)cc1)C1CCCCC1]

In some embodiments, DBM is

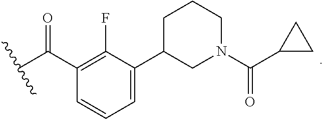 [SMILES: *C(=O)c1cccc(C2CCCN(C(=O)C3CC3)C2)c1F]

In some embodiments, DBM is

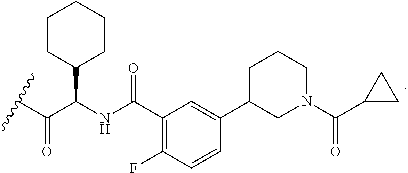 [SMILES: *C(=O)[C@H](NC(=O)c1cc(C2CCCN(C(=O)C3CC3)C2)ccc1F)C1CCCCC1]

In some embodiments, DBM is

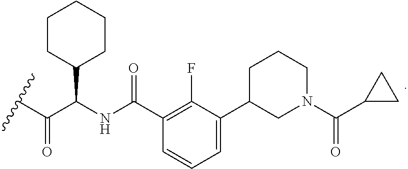 [SMILES: *C(=O)[C@H](NC(=O)c1cccc(C2CCCN(C(=O)C3CC3)C2)c1F)C1CCCCC1]

In some embodiments, DBM is

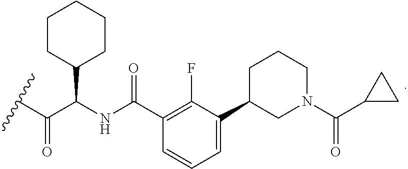 [SMILES: *C(=O)[C@H](NC(=O)c1cccc([C@H]2CCCN(C(=O)C3CC3)C2)c1F)C1CCCCC1]

In some embodiments, DBM is

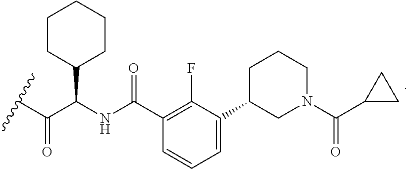 [SMILES: *C(=O)[C@H](NC(=O)c1cccc([C@@H]2CCCN(C(=O)C3CC3)C2)c1F)C1CCCCC1]

In some embodiments, DBM is

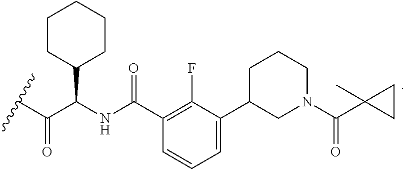 [SMILES: *C(=O)[C@H](NC(=O)c1cccc(C2CCCN(C(=O)C3(C)CC3)C2)c1F)C1CCCCC1]

In some embodiments, DBM is

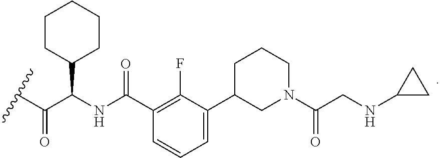 [SMILES: *C(=O)[C@H](NC(=O)c1cccc(C2CCCN(C(=O)CNC3CC3)C2)c1F)C1CCCCC1]

In some embodiments, DBM is

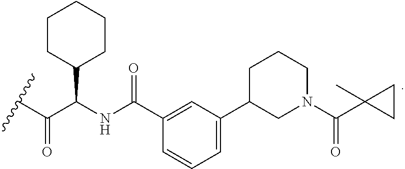 [SMILES: *C(=O)[C@H](NC(=O)c1cccc(C2CCCN(C(=O)C3(C)CC3)C2)c1)C1CCCCC1]

In some embodiments, DBM is

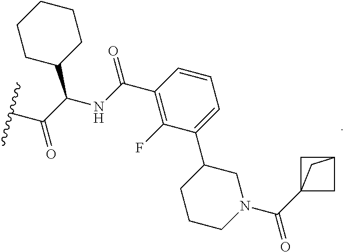 [SMILES: *C(=O)[C@H](NC(=O)c1cccc(C2CCCN(C(=O)C34CC(C3)C4)C2)c1F)C1CCCCC1]

In some embodiments, DBM is

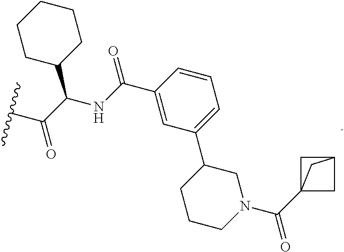 [SMILES: *C(=O)[C@H](NC(=O)c1cccc(C2CCCN(C(=O)C34CC(C3)C4)C2)c1)C1CCCCC1]

In some embodiments, DBM is

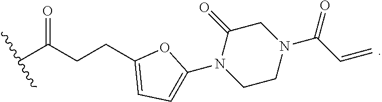 [SMILES: *C(=O)CCc1ccc(N2CCN(C(=O)C=C)CC2=O)o1]

In some embodiments, DBM is

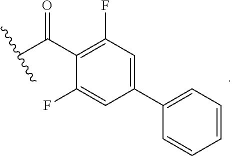 [SMILES: *C(=O)c1c(F)cc(-c2ccccc2)cc1F]

In some embodiments, DBM is

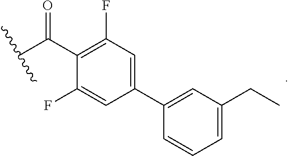 [SMILES: *C(=O)c1c(F)cc(-c2cccc(CC)c2)cc1F]

In some embodiments, DBM is

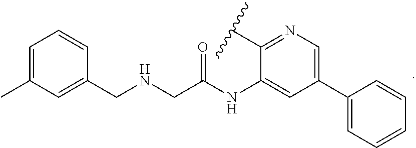 [SMILES: *c1ncc(-c2ccccc2)cc1NC(=O)CNCc1cccc(C)c1]

In some embodiments, DBM is

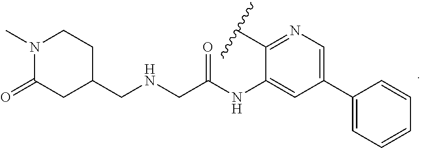 [SMILES: *c1ncc(-c2ccccc2)cc1NC(=O)CNCC1CCN(C)C(=O)C1]

In some embodiments, DBM is

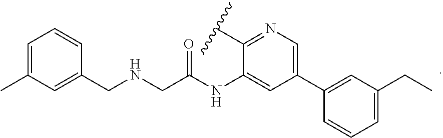 [SMILES: *c1ncc(-c2cccc(CC)c2)cc1NC(=O)CNCc1cccc(C)c1]

In some embodiments, DBM is

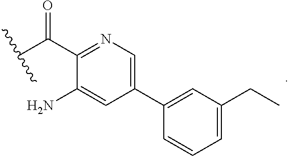 [SMILES: *C(=O)c1ncc(-c2cccc(CC)c2)cc1N]

In some embodiments, DBM is

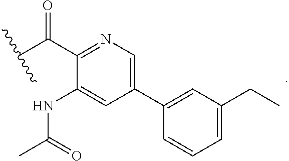 [SMILES: *C(=O)c1ncc(-c2cccc(CC)c2)cc1NC(C)=O]

In some embodiments, DBM is

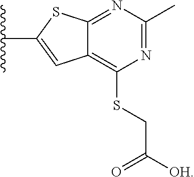 [SMILES: *c1cc2c(SCC(=O)O)nc(C)nc2s1]

In some embodiments, DBM is

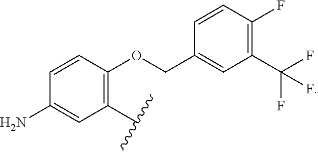 [SMILES: *c1cc(N)ccc1OCc1ccc(F)c(C(F)(F)F)c1]

In some embodiments, DBM is

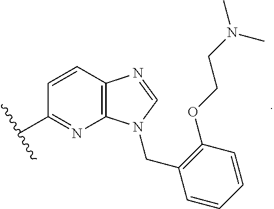 [SMILES: *c1ccc2ncn(Cc3ccccc3OCCN(C)C)c2n1]

In some embodiments, DBM is

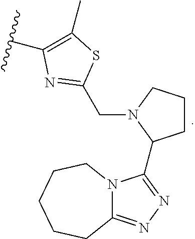 [SMILES: *c1nc(CN2CCCC2c2nnc3n2CCCCC3)sc1C]

In some embodiments, DBM is

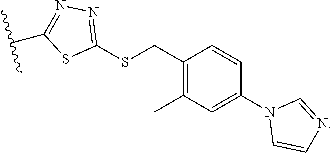 [SMILES: *c1nnc(SCc2ccc(-n3ccnc3)cc2C)s1]

In some embodiments, DBM is

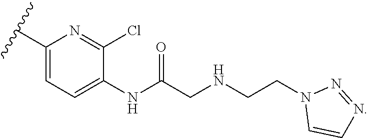 [SMILES: *c1ccc(NC(=O)CNCCn2ccnn2)c(Cl)n1]

In some embodiments, DBM is

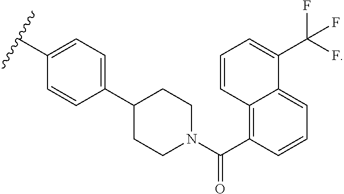 [SMILES: *c1ccc(C2CCN(C(=O)c3cccc4c(C(F)(F)F)cccc34)CC2)cc1]

In some embodiments, DBM is

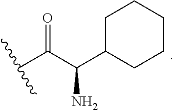 [SMILES: *C(=O)[C@H](N)C1CCCCC1]

In some embodiments, DBM is

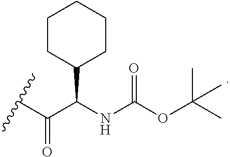 [SMILES: *C(=O)[C@H](NC(=O)OC(C)(C)C)C1CCCCC1]

In some embodiments, DBM is

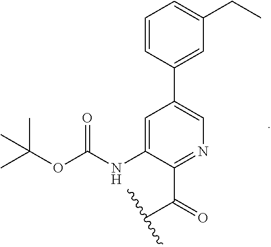 [SMILES: *C(=O)c1ncc(-c2cccc(CC)c2)cc1NC(=O)OC(C)(C)C]

In some embodiments, DBM is

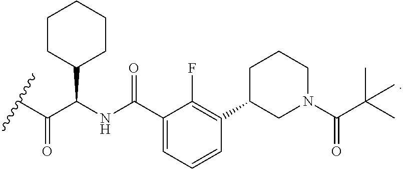 [SMILES: *C(=O)[C@H](NC(=O)c1cccc([C@@H]2CCCN(C(=O)C(C)(C)C)C2)c1F)C1CCCCC1]

In some embodiments, DBM is

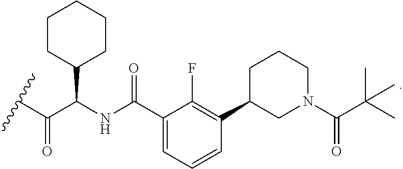 [SMILES: *C(=O)[C@H](NC(=O)c1cccc([C@H]2CCCN(C(=O)C(C)(C)C)C2)c1F)C1CCCCC1]

In some embodiments, DBM is

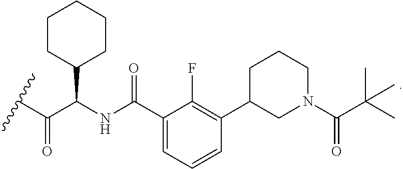 [SMILES: *C(=O)[C@H](NC(=O)c1cccc(C2CCCN(C(=O)C(C)(C)C)C2)c1F)C1CCCCC1]

In some embodiments, DBM is selected from those depicted in Table 1, below.

In some embodiments, DBM is selected from those depicted in Table A, below.

### Linker (L)

As defined above and described herein, L is a bivalent moiety that connects PBM to DIM (e.g., DBM). In some embodiments, L is a bivalent moiety that connects PBM to DIM. In some embodiments, L is a bivalent moiety that connects PBM to DBM.

In some embodiments, L is a covalent bond or a bivalent, saturated or partially unsaturated, straight or branched C₁₋₅₀ hydrocarbon chain, wherein 0-6 methylene units of L are independently replaced by -Cy-, —O—, —NR—, —SiR₂—, —Si(OH)R—, —Si (OH)₂—, —P(O)OR—, —P(O)R—, —P(O)NR₂—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, —NRC(O)O—,

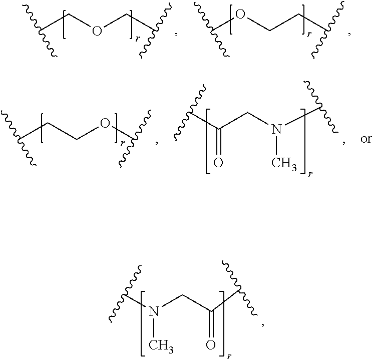

wherein:
each -Cy- is independently an optionally substituted bivalent ring selected from phenylenyl, an 8-10 membered bicyclic arylenyl, a 4-7 membered saturated or partially unsaturated carbocyclylenyl, a 6-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 6-11 membered saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, and an 8-10 membered bicyclic heteroarylenyl having 1-5 heteroatoms independently selected from nitrogen, oxygen, and sulfur;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or;
   two R groups on the same nitrogen are optionally taken together with their intervening atoms to form a 4-7 membered saturated, partially unsaturated, or heteroaryl ring having 0-3 heteroatoms, in addition to the nitrogen, independently selected from nitrogen, oxygen, and sulfur, and;
r is 0, 1, 2, 3, 4, 5, 6, 7, 8, 9, or 10.

In some embodiments, each -Cy- is independently an optionally substituted bivalent phenylenyl. In some embodiments, each -Cy- is independently an optionally substituted 8-10 membered bicyclic arylenyl. In some embodiments, each -Cy- is independently an optionally substituted 4-7 membered saturated or partially unsaturated carbocyclylenyl. In some embodiments, each -Cy- is independently an optionally substituted 6-11 membered saturated or partially unsaturated spiro carbocyclylenyl. In some embodiments, each -Cy- is independently an optionally substituted 8-10 membered bicyclic saturated or partially unsaturated carbocyclylenyl. In some embodiments, each -Cy- is independently an optionally substituted 4-7 membered saturated or partially unsaturated heterocyclylenyl having 1-2 heteroatoms independently selected from nitrogen, oxygen, and sulfur. In some embodiments, each -Cy- is independently an optionally substituted 6-11 membered saturated or partially unsaturated spiro heterocyclylenyl having 1-2 heteroatoms independently selected from nitrogen, oxygen, and sulfur. In some embodiments, each -Cy- is independently an optionally substituted 8-10 membered bicyclic saturated or partially unsaturated heterocyclylenyl having 1-2 heteroatoms independently selected from nitrogen, oxygen, and sulfur. In some embodiments, each -Cy- is independently an optionally substituted 5-6 membered heteroarylenyl having 1-4 heteroatoms independently selected from nitrogen, oxygen, and sulfur. In some embodiments, each -Cy- is independently an optionally substituted 8-10 membered bicyclic heteroarylenyl having 1-5 heteroatoms independently selected from nitrogen, oxygen, and sulfur.

In some embodiments, -Cy- is substituted with C₁₋₆ alkyl (e.g., methyl, ethyl, isopropyl). In some embodiments, -Cy- is substituted with oxo. In some embodiments, -Cy- is substituted with halogen. In some embodiments, -Cy- is substituted with fluoro. In some embodiments, -Cy- is substituted with geminal difluoro. In some embodiments, -Cy- is substituted with —OH. In some embodiments, -Cy- is substituted with —NR₂.

In some embodiments, -Cy- is selected from those depicted in Table 1, below.

In some embodiments, -Cy- is selected from those depicted in Table B, below.

In some embodiments, r is 0. In some embodiments, r is 1. In some embodiments, r is 2. In some embodiments, r is 3. In some embodiments, r is 4. In some embodiments, r is 5. In some embodiments, r is 6. In some embodiments, r is 7. In some embodiments, r is 8. In some embodiments, r is 9. In some embodiments, r is 10.

In some embodiments, r is selected from those depicted in Table 1, below.

In some embodiments, r is selected from those depicted in Table B, below.

In some embodiments, L is —O(CH₂)₁₋₁₀—O—. In some embodiments, L is —O(CH₂)₁₋₁₀—NR—. In some embodiments, L is —O(CH₂)₁₋₁₀—C(O)—. In some embodiments, L is —O(CH₂)₁₋₁₀—OCH₂CH₂NR—. In some embodiments, L is —O(CH₂)₁₋₁₀—NRCH₂C(O)—. In some embodiments, L is —O(CH₂)₁₋₁₀—CH₂CH₂OCH₂C(O)—. In some embodiments, L is —O(CH₂)₁₋₁₀—(OCH₂CH₂)₁₋₁₀—NR—. In some embodiments, L is —O(CH₂)₁₋₁₀—CH₂C(O)NRCH₂CH₂NR—. In some embodiments, L is —O(CH₂)₁₋₁₀—NRCH₂C(O)NRCH₂CH₂NR—. In some embodiments, L is —NR(CH₂)₁₋₁₀—O—. In some embodiments, L is —NR(CH₂)₁₋₁₀—NR—. In some embodiments, L is —NR(CH₂)₁₋₁₀—C(O)—. In some embodiments, L is —NR(CH₂)₁₋₁₀—OCH₂CH₂NR—. In some embodiments, L is —NR(CH₂)₁₋₁₀—NRCH₂C(O)—. In some embodiments, L is —NR(CH₂)₁₋₁₀—CH₂CH₂OCH₂C(O)—. In some embodiments, L is —NR(CH₂)₁₋₁₀—(OCH₂CH₂)₁₋₁₀—NR—. In some embodiments, L is —NR(CH₂)₁₋₁₀—CH₂C(O)NRCH₂CH₂NR—. In some embodiments, L is —NR(CH₂)₁₋₁₀—NRCH₂C(O)NRCH₂CH₂NR—. In some embodiments, L is —C(O)(CH₂)₁₋₁₀—O—. In some embodiments, L is —C(O)(CH₂)₁₋₁₀—NR—. In some embodiments, L is —C(O)(CH₂)₁₋₁₀—C(O)—. In some embodiments, L is —C(O)(CH₂)₁₋₁₀—OCH₂CH₂NR—. In some embodiments, L is —C(O)(CH₂)₁₋₁₀—NRCH₂C(O)—. In some embodiments, L is —C(O)(CH₂)₁₋₁₀—CH₂CH₂OCH₂C(O)—. In some embodiments, L is —C(O)(CH₂)₁₋₁₀—(OCH₂CH₂)₁₋₁₀—NR—. In some embodiments, L is —C(O)(CH₂)₁₋₁₀—CH₂C(O)NRCH₂CH₂NR—. In some embodiments, L is —C(O)(CH₂)₁₋₁₀—NRCH₂C(O)NRCH₂CH₂NR—. In some embodiments, L is —NRCH₂CH₂O(CH₂)₁₋₁₀—O—. In some embodiments, L is —NRCH₂CH₂O(CH₂)₁₋₁₀—NR—. In some embodiments, L is —NRCH₂CH₂O(CH₂)₁₋₁₀—C(O)—. In some embodiments, L is —NRCH₂CH₂O(CH₂)₁₋₁₀—OCH₂CH₂NR—. In some embodiments, L is —NRCH₂CH₂O(CH₂)₁₋₁₀—NRCH₂C(O)—. In some embodiments, L is —NRCH₂CH₂O(CH₂)₁₋₁₀—CH₂CH₂OCH₂C(O)—. In some embodiments, L is —NRCH₂CH₂O(CH₂)₁₋₁₀—(OCH₂CH₂)₁₋₁₀—NR—. In some embodiments, L is —NRCH₂CH₂O(CH₂)₁₋₁₀—CH₂C(O)NRCH₂CH₂NR—. In some embodiments, L is —NRCH₂CH₂O(CH₂)₁₋₁₀—NRCH₂C(O)NRCH₂CH₂NR—. In some embodiments, L is —C(O)CH₂NR(CH₂)₁₋₁₀—O—. In some embodiments, L is —C(O)CH₂NR(CH₂)₁₋₁₀—NR—. In some embodiments, L is —C(O)CH₂NR(CH₂)₁₋₁₀—C(O)—. In some embodiments, L is —C(O)CH₂NR(CH₂)₁₋₁₀—OCH₂CH₂NR—. In some embodiments, L is —C(O)CH₂NR(CH₂)₁₋₁₀—NRCH₂C(O)—. In some embodiments, L is —C(O)CH₂NR(CH₂)₁₋₁₀—CH₂CH₂OCH₂C(O)—. In some embodiments, L is —C(O)CH₂NR(CH₂)₁₋₁₀—(OCH₂CH₂)₁₋₁₀—NR—. In some embodiments, L is —C(O)CH₂NR(CH₂)₁₋₁₀—CH₂C(O)NRCH₂CH₂NR—. In some embodiments, L is —C(O)CH₂NR(CH₂)₁₋₁₀—NRCH₂C(O)NRCH₂CH₂NR—. In some embodiments, L is —C(O)CH₂OCH₂CH₂(CH₂)₁₋₁₀—O—. In some embodiments, L is —C(O)CH₂OCH₂CH₂(CH₂)₁₋₁₀—NR—. In some embodiments, L is —C(O)CH₂OCH₂CH₂(CH₂)₁₋₁₀—C(O)—. In some embodiments, L is —C(O)CH₂OCH₂CH₂(CH₂)₁₋₁₀—OCH₂CH₂NR—. In some embodiments, L is —C(O)CH₂OCH₂CH₂(CH₂)₁₋₁₀—NRCH₂C(O)—. In some embodiments, L is —C(O)CH₂OCH₂CH₂(CH₂)₁₋₁₀—CH₂CH₂OCH₂C(O)—. In some embodiments, L is —C(O)CH₂OCH₂CH₂(CH₂)₁₋₁₀—(OCH₂CH₂)₁₋₁₀—NR—. In some embodiments, L is —C(O)CH₂OCH₂CH₂(CH₂)₁₋₁₀—CH₂C(O)NRCH₂CH₂NR—. In some embodiments, L is —C(O)CH₂OCH₂CH₂(CH₂)₁₋₁₀—NRCH₂C(O)NRCH₂CH₂NR—. In some embodiments, L is —NR(CH₂CH₂O)₁₋₁₀—(CH₂)₁₋₁₀—O—. In some embodiments, L is —NR(CH₂CH₂O)₁₋₁₀—(CH₂)₁₋₁₀—NR—. In some embodiments, L is —NR(CH₂CH₂O)₁₋₁₀—(CH₂)₁₋₁₀—C(O)—. In some embodiments, L is —NR(CH₂CH₂O)₁₋₁₀—(CH₂)₁₋₁₀—OCH₂CH₂NR—. In some embodiments, L is —NR(CH₂CH₂O)₁₋₁₀—(CH₂)₁₋₁₀—NRCH₂C(O)—. In some embodiments, L is —NR(CH₂CH₂O)₁₋₁₀—(CH₂)₁₋₁₀—CH₂CH₂OCH₂C(O)—. In some embodiments, L is —NR(CH₂CH₂O)₁₋₁₀—(CH₂)₁₋₁₀—(OCH₂CH₂)₁₋₁₀—NR—. In some embodiments, L is —NR(CH₂CH₂O)₁₋₁₀—(CH₂)₁₋₁₀—CH₂C(O)NRCH₂CH₂NR—. In some embodiments, L is —NR(CH₂CH₂O)₁₋₁₀—(CH₂)₁₋₁₀—NRCH₂C(O)NRCH₂CH₂NR—. In some embodiments, L is —NRCH₂CH₂NRC(O)CH₂(CH₂)₁₋₁₀—O—. In some embodiments, L is —NRCH₂CH₂NRC(O)CH₂(CH₂)₁₋₁₀—NR—. In some embodiments, L is —NRCH₂CH₂NRC(O)CH₂(CH₂)₁₋₁₀—C(O)—. In some embodiments, L is —NRCH₂CH₂NRC(O)CH₂(CH₂)₁₋₁₀—NR—. In some embodiments, L is —NRCH₂CH₂NRC(O)CH₂(CH₂)₁₋₁₀—NRCH₂C(O)—. In some embodiments, L is —NRCH₂CH₂NRC(O)CH₂(CH₂)₁₋₁₀—CH₂CH₂OCH₂C(O)—. In some embodiments, L is —NRCH₂CH₂NRC(O)CH₂(CH₂)₁₋₁₀—(OCH₂CH₂)₁₋₁₀—NR—. In some embodiments, L is —NRCH₂CH₂NRC(O)CH₂(CH₂)₁₋₁₀—CH₂C(O)NRCH₂CH₂NR—. In some embodiments, L is —NRCH₂CH₂NRC(O)CH₂(CH₂)₁₋₁₀—NRCH₂C(O)NRCH₂CH₂NR—. In some embodiments, L is —NRCH₂CH₂NRC(O)CH₂NR(CH₂)₁₋₁₀—O—. In some embodiments, L is —NRCH₂CH₂NRC(O)CH₂NR(CH₂)₁₋₁₀—NR—. In some embodiments, L is —NRCH₂CH₂NRC(O)CH₂NR(CH₂)₁₋₁₀—C(O)—. In some embodiments, L is —NRCH₂CH₂NRC(O)CH₂NR(CH₂)₁₋₁₀—OCH₂CH₂NR—. In some embodiments, L is —NRCH₂CH₂NRC(O)CH₂NR(CH₂)₁₋₁₀—NRCH₂C(O)—. In some embodiments, L is —NRCH₂CH₂NRC(O)CH₂NR(CH₂)₁₋₁₀—CH₂CH₂OCH₂C(O)—. In some embodiments, L is —NRCH₂CH₂NRC(O)CH₂NR(CH₂)₁₋₁₀—(OCH₂CH₂)₁₋₁₀—NR—. In some embodiments, L is —NRCH₂CH₂NRC(O)CH₂NR(CH₂)₁₋₁₀—CH₂C(O)NRCH₂CH₂NR—. In some embodiments, L is —NRCH₂CH₂NRC(O)CH₂NR(CH₂)₁₋₁₀—NRCH₂C(O)NRCH₂CH₂NR—.

In some embodiments, L is —(OCH₂CH₂)₁₋₁₀—O—. In some embodiments, L is —(OCH₂CH₂)₁₋₁₀-Cy-. In some embodiments, L is —(OCH₂CH₂)₁₋₁₀—NR—. In some embodiments, L is —(OCH₂CH₂)₁₋₁₀—C(O)—. In some embodiments, L is —(OCH₂CH₂)₁₋₁₀—NRCH₂C(O)—. In some embodiments, L is —(OCH₂CH₂)₁₋₁₀—NR—. In some embodiments, L is —(OCH₂CH₂)₁₋₁₀—NRCH₂C(O)NRCH₂CH₂NR—. In some embodiments, L is —NRCH₂CH₂(OCH₂CH₂)₁₋₁₀—O—. In some embodiments, L is —NRCH₂CH₂(OCH₂CH₂)₁₋₁₀-Cy-. In some embodiments, L is —NRCH₂CH₂(OCH₂CH₂)₁₋₁₀—NR—. In some embodiments, L is —NRCH₂CH₂(OCH₂CH₂)₁₋₁₀—C(O)—. In some embodiments, L is —NRCH₂CH₂(OCH₂CH₂)₁₋₁₀—NRCH₂C(O)—. In some embodiments, L is —NRCH₂CH₂(OCH₂CH₂)₁₋₁₀—NR—. In some embodiments, L is —NRCH₂CH₂(OCH₂CH₂)₁₋₁₀—NRCH₂C(O)NRCH₂CH₂NR—. In some embodiments, L is —C(O)CH₂CH₂(OCH₂CH₂)₁₋₁₀—O—. In some embodiments, L is —C(O)CH₂CH₂(OCH₂CH₂)₁₋₁₀-Cy-. In some embodiments, L is —C(O)CH₂CH₂(OCH₂CH₂)₁₋₁₀—NR—. In some embodiments, L is —C(O)CH₂CH₂(OCH₂CH₂)₁₋₁₀—C(O)—. In some embodiments, L is —C(O)CH₂CH₂(OCH₂CH₂)₁₋₁₀—NRCH₂C(O)—. In some embodiments, L is —C(O)CH₂CH₂(OCH₂CH₂)₁₋₁₀—NR—. In some embodiments, L is —C(O)CH₂CH₂(OCH₂CH₂)₁₋₁₀—NRCH₂C(O)NRCH₂CH₂NR—. In some embodiments, L is —C(O)CH₂OCH₂CH₂(OCH₂CH₂)₁₋₁₀—O—. In some embodiments, L is —C(O)CH₂OCH₂CH₂(OCH₂CH₂)₁₋₁₀-Cy-. In some embodiments, L is —C(O)CH₂OCH₂CH₂(OCH₂CH₂)₁₋₁₀—NR—. In some embodiments, L is —C(O)CH₂OCH₂CH₂(OCH₂CH₂)₁₋₁₀—C(O)—. In some embodiments, L is —C(O)CH₂OCH₂CH₂(OCH₂CH₂)₁₋₁₀—NRCH₂C(O)—. In some embodiments, L is —C(O)CH₂OCH₂CH₂(OCH₂CH₂)₁₋₁₀—NR—. In some embodiments, L is —C(O)CH₂OCH₂CH₂(OCH₂CH₂)₁₋₁₀—NRCH₂C(O)NRCH₂CH₂NR—. In some embodiments, L is —C(O)CH₂NRCH₂CH₂(OCH₂CH₂)₁₋₁₀—O—. In some embodiments, L is —C(O)CH₂NRCH₂CH₂(OCH₂CH₂)₁₋₁₀-Cy-. In some embodiments, L is —C(O)CH₂NRCH₂CH₂(OCH₂CH₂)₁₋₁₀—NR—. In some embodiments, L is —C(O)CH₂NRCH₂CH₂(OCH₂CH₂)₁₋₁₀—C(O)—. In some embodiments, L is —C(O)CH₂NRCH₂CH₂(OCH₂CH₂)₁₋₁₀—NRCH₂C(O)—. In some embodiments, L is —C(O)CH₂NRCH₂CH₂(OCH₂CH₂)₁₋₁₀—NR—. In some embodiments, L is —C(O)CH₂NRCH₂CH₂(OCH₂CH₂)₁₋₁₀—NRCH₂C(O)NRCH₂CH₂NR—. In some embodiments, L is —NRCH₂CH₂NRC(O)CH₂(OCH₂CH₂)₁₋₁₀—O—. In some embodiments, L is —NRCH₂CH₂NRC(O)CH₂(OCH₂CH₂)₁₋₁₀-Cy-. In some embodiments, L is —NRCH₂CH₂NRC(O)CH₂(OCH₂CH₂)₁₋₁₀—NR—. In some embodiments, L is —NRCH₂CH₂NRC(O)CH₂(OCH₂CH₂)₁₋₁₀—C(O)—. In some embodiments, L is —NRCH₂CH₂NRC(O)CH₂(OCH₂CH₂)₁₋₁₀—NRCH₂C(O)—. In some embodiments, L is —NRCH₂CH₂NRC(O)CH₂(OCH₂CH₂)₁₋₁₀—NR—. In some embodiments, L is —NRCH₂CH₂NRC(O)CH₂(OCH₂CH₂)₁₋₁₀—NRCH₂C(O)NRCH₂CH₂NR—. In some embodiments, L is —NRCH₂CH₂NRC(O)CH₂NRCH₂CH₂(OCH₂CH₂)₁₋₁₀—O—. In some embodiments, L is —NRCH₂CH₂NRC(O)CH₂NRCH₂CH₂(OCH₂CH₂)₁₋₁₀-Cy-. In some embodiments, L is —NRCH₂CH₂NRC(O)CH₂NRCH₂CH₂(OCH₂CH₂)₁₋₁₀—NR—. In some embodiments, L is —NRCH₂CH₂NRC(O)CH₂NRCH₂CH₂(OCH₂CH₂)₁₋₁₀—C(O)—. In some embodiments, L is —NRCH₂CH₂NRC(O)CH₂NRCH₂CH₂(OCH₂CH₂)₁₋₁₀—NRCH₂C(O)—. In some embodiments, L is —NRCH₂CH₂NRC(O)CH₂NRCH₂CH₂(OCH₂CH₂)₁₋₁₀—NR—. In some embodiments, L is —NRCH₂CH₂NRC(O)CH₂NRCH₂CH₂(OCH₂CH₂)₁₋₁₀—NRCH₂C(O)NRCH₂CH₂NR—. In some embodiments, L is —O(CH₂CH₂O)₁₋₁₀—CH₂CH₂O—. In some embodiments, L is -Cy-(CH₂CH₂O)₁₋₁₀—CH₂CH₂O—. In some embodiments, L is —NR(CH₂CH₂O)₁₋₁₀—CH₂CH₂O—. In some embodiments, L is —C(O)(CH₂CH₂O)₁₋₁₀—CH₂CH₂O—. In some embodiments, L is —C(O)CH₂NR(CH₂CH₂O)₁₋₁₀—CH₂CH₂O—. In some embodiments, L is —NRCH₂CH₂O(CH₂CH₂O)₁₋₁₀—CH₂CH₂O—. In some embodiments, L is —NRCH₂CH₂NRC(O)CH₂NR(CH₂CH₂O)₁₋₁₀—CH₂CH₂O—. In some embodiments, L is —O(CH₂CH₂O)₁₋₁₀—CH₂CH₂NR—. In some embodiments, L is -Cy-(CH₂CH₂O)₁₋₁₀—CH₂CH₂NR—. In some embodiments, L is —NR(CH₂CH₂O)₁₋₁₀—CH₂CH₂NR—. In some embodiments, L is —C(O)(CH₂CH₂O)₁₋₁₀—CH₂CH₂NR—. In some embodiments, L is —C(O)CH₂NR(CH₂CH₂O)₁₋₁₀—CH₂CH₂NR—. In some embodiments, L is —NRCH₂CH₂O(CH₂CH₂O)₁₋₁₀—CH₂CH₂NR—. In some embodiments, L is —NRCH₂CH₂NRC(O)CH₂NR(CH₂CH₂O)₁₋₁₀—CH₂CH₂NR—. In some embodiments, L is —O(CH₂CH₂O)₁₋₁₀—CH₂CH₂C(O)—. In some embodiments, L is -Cy-(CH₂CH₂O)₁₋₁₀—CH₂CH₂C(O)—. In some embodiments, L is —NR(CH₂CH₂O)₁₋₁₀—CH₂CH₂C(O)—. In some embodiments, L is —C(O)(CH₂CH₂O)₁₋₁₀—CH₂CH₂C(O)—. In some embodiments, L is —C(O)CH₂NR(CH₂CH₂O)₁₋₁₀—CH₂CH₂C(O)—. In some embodiments, L is —NRCH₂CH₂O(CH₂CH₂O)₁₋₁₀—CH₂CH₂C(O)—. In some embodiments, L is —NRCH₂CH₂NRC(O)CH₂NR(CH₂CH₂O)₁₋₁₀—CH₂CH₂C(O)—. In some embodiments, L is —O(CH₂CH₂O)₁₋₁₀—CH₂CH₂OCH₂C(O)—. In some embodiments, L is -Cy-(CH₂CH₂O)₁₋₁₀—CH₂CH₂OCH₂C(O)—. In some embodiments, L is —NR(CH₂CH₂O)₁₋₁₀—CH₂CH₂OCH₂C(O)—. In some embodiments, L is —C(O)(CH₂CH₂O)₁₋₁₀—CH₂CH₂OCH₂C(O)—. In some embodiments, L is —C(O)CH₂NR(CH₂CH₂O)₁₋₁₀—CH₂CH₂OCH₂C(O)—. In some embodiments, L is —NRCH₂CH₂O(CH₂CH₂O)₁₋₁₀—CH₂CH₂OCH₂C(O)—. In some embodiments, L is —NRCH₂CH₂NRC(O)CH₂NR(CH₂CH₂O)₁₋₁₀—CH₂CH₂OCH₂C(O)—. In some embodiments, L is —O(CH₂CH₂O)₁₋₁₀—CH₂CH₂NRCH₂C(O)—. In some embodiments, L is -Cy-(CH₂CH₂O)₁₋₁₀—CH₂CH₂NRCH₂C(O)—. In some embodiments, L is —NR(CH₂CH₂O)₁₋₁₀—CH₂CH₂NRCH₂C(O)—. In some embodiments, L is —C(O)(CH₂CH₂O)₁₋₁₀—CH₂CH₂NRCH₂C(O)—. In some embodiments, L is —C(O)CH₂NR(CH₂CH₂O)₁₋₁₀—CH₂CH₂NRCH₂C(O)—. In some embodiments, L is —NRCH₂CH₂O(CH₂CH₂O)₁₋₁₀—CH₂CH₂NRCH₂C(O)—. In some embodiments, L is —NRCH₂CH₂NRC(O)CH₂NR(CH₂CH₂O)₁₋₁₀—CH₂CH₂NRCH₂C(O)—. In some embodiments, L is —O(CH₂CH₂O)₁₋₁₀—CH₂C(O)NRCH₂CH₂NR—. In some embodiments, L is -Cy-(CH₂CH₂O)₁₋₁₀—CH₂C(O)NRCH₂CH₂NR—. In some embodiments, L is —NR(CH₂CH₂O)₁₋₁₀—CH₂C(O)NRCH₂CH₂NR—. In some embodiments, L is —C(O)(CH₂CH₂O)₁₋₁₀—CH₂C(O)NRCH₂CH₂NR—. In some embodiments, L is —C(O)CH₂NR(CH₂CH₂O)₁₋₁₀—CH₂C(O)NRCH₂CH₂NR—. In some embodiments, L is —NRCH₂CH₂O(CH₂CH₂O)₁₋₁₀—CH₂C(O)NRCH₂CH₂NR—. In some embodiments, L is —NRCH₂CH₂NRC(O)CH₂NR(CH₂CH₂O)₁₋₁₀—CH₂C(O)NRCH₂CH₂NR—. In some embodiments, L is —O(CH₂CH₂O)₁₋₁₀—CH₂CH₂NRCH₂C(O)NRCH₂CH₂NR—. In some embodiments, L is -Cy-(CH₂CH₂O)₁₋₁₀—CH₂CH₂NRCH₂C(O)NRCH₂CH₂NR—. In some embodiments, L is —NR(CH₂CH₂O)₁₋₁₀—CH₂CH₂NRCH₂C(O)NRCH₂CH₂NR—. In some embodiments, L is —C(O)(CH₂CH₂O)₁₋₁₀—CH₂CH₂NRCH₂C(O)NRCH₂CH₂NR—. In some embodiments, L is —C(O)CH₂NR(CH₂CH₂O)₁₋₁₀—CH₂CH₂NRCH₂C(O)NRCH₂CH₂NR—. In some embodiments, L is —NRCH₂CH₂O(CH₂CH₂O)₁₋₁₀—CH₂CH₂NRCH₂C(O)NRCH₂CH₂NR—. In some embodiments, L is —NRCH₂CH₂NRC(O)CH₂NR(CH₂CH₂O)₁₁₀—CH₂CH₂NRCH₂C(O)NRCH₂CH₂NR—.

In some embodiments, L is —NRCH₂CH₂-Cy-CH₂O—. In some embodiments, L is —OCH₂-Cy-CH₂CH₂NR—. In some embodiments, L is —NRCH₂CH₂-Cy-CH₂CH₂NR—. In some embodiments, L is —NRCH₂CH₂-Cy-CH₂C(O)—. In some embodiments, L is —C(O)CH₂-Cy-CH₂CH₂NR—. In some embodiments, L is —C(O)CH₂NRCH₂CH₂-Cy-CH₂NR—. In some embodiments, L is —NRCH₂-Cy-CH₂CH₂NRCH₂C(O)—. In some embodiments, L is —C(O)CH₂CH₂-Cy-CH₂C(O)—. In some embodiments, L is —C(O)CH₂-Cy-CH₂CH₂C(O)—. In some embodiments, L is —C(O)CH₂CH₂-Cy-CH₂CH₂NR—. In some embodiments, L is —NRCH₂CH₂-Cy-CH₂CH₂C(O)—. In some embodiments, L is —NRCH₂CH₂NRC(O)CH₂NRCH₂CH₂-Cy-CH₂NR—. In some embodiments, L is —NRCH₂-Cy-CH₂CH₂NRCH₂C(O)NRCH₂CH₂NR—. In some embodiments, L is —NRCH₂CH₂-Cy-SO₂NR—. In some embodiments, L is —NRSO₂-Cy-CH₂CH₂NR—. In some embodiments, L is -Cy-(OCH₂CH₂)₁₋₁₀—NR—. In some embodiments, L is -Cy-(OCH₂CH₂)₁₋₁₀—O—. In some embodiments, L is —O(CH₂CH₂O)₁₋₁₀-Cy-. In some embodiments, L is —NR(CH₂CH₂O)₁₋₁₀-Cy-. In some embodiments, L is —(OCH₂CH₂)₁₋₁₀-Cy-C(O)—. In some embodiments, L is —C(O)-Cy-(CH₂CH₂O)₁₋₁₀—. In some embodiments, L is —NR(CH₂CH₂O)₁₋₁₀-Cy-NRCH₂C(O)—. In some embodiments, L is —C(O)CH₂NR-Cy-(OCH₂CH₂)₁₋₁₀—NR—. In some embodiments, L is —C(O)CH₂-Cy-NRCH₂C(O)—. In some embodiments, L is —C(O)CH₂NR-Cy-CH₂C(O)—. In some embodiments, L is —NRCH₂CH₂NRC(O)CH₂-Cy-C(O)—. In some embodiments, L is —C(O)-Cy-CH₂C(O)NRCH₂CH₂NR—. In some embodiments, L is —NR(CH₂CH₂O)₁₋₁₀-Cy-CH₂CH₂C(O)—. In some embodiments, L is —C(O)CH₂CH₂-Cy-(OCH₂CH₂)₁₋₁₀—NR—. In some embodiments, L is —OCH₂CH₂-Cy-O—. In some embodiments, L is —O-Cy-CH₂CH₂O—. In some embodiments, L is —NRCH₂CH₂O-Cy-NR—. In some embodiments, L is —NR-Cy-OCH₂CH₂NR—. In some embodiments, L is —C(O)CH₂-Cy-C(O)—. In some embodiments, L is —C(O)-Cy-CH₂C(O)—. In some embodiments, L is —NRCH₂CH₂O-Cy-NRCH₂C(O)NRCH₂CH₂NR—. In some embodiments, L is —NRCH₂CH₂NRC(O)CH₂NR-Cy-OCH₂CH₂NR—. In some embodiments, L is —NRCH₂CH₂NRC(O)CH₂-Cy-NRCH₂C(O)NRCH₂CH₂NR—. In some embodiments, L is —NRCH₂CH₂NRC(O)CH₂NR-Cy-CH₂C(O)NRCH₂CH₂NR—.

In some embodiments, L is -Cy-CH₂-Cy-. In some embodiments, L is -Cy-CH₂-Cy-CH₂C(O)—. In some embodiments, L is —C(O)CH₂-Cy-CH₂-Cy-. In some embodiments, L is -Cy-CH₂-Cy-CH₂CH₂O—. In some embodiments, L is —OCH₂CH₂-Cy-CH₂-Cy-. In some embodiments, L is -Cy-CH₂-Cy-CH₂CH₂NR—. In some embodiments, L is —NRCH₂CH₂-Cy-CH₂-Cy-. In some embodiments, L is -Cy-CH₂-Cy-CH₂C(O)NRCH₂CH₂NR—. In some embodiments, L is —NRCH₂CH₂NRC(O)CH₂-Cy-CH₂-Cy-. In some embodiments, L is —O—CH₂CH₂-Cy-CH₂-Cy-. In some embodiments, L is —C(O)CH₂-Cy-CH₂-Cy-. In some embodiments, L is —C(O)CH₂-Cy-CH₂-Cy-CH₂C(O)—. In some embodiments, L is —C(O)CH₂-Cy-CH₂-Cy-CH₂CH₂OCH₂C(O)—. In some embodiments, L is —C(O)CH₂OCH₂CH₂-Cy-CH₂-Cy-CH₂C(O)—. In some embodiments, L is —NRCH₂CH₂NRC(O)CH₂-Cy-CH₂-Cy-. In some embodiments, L is -Cy-CH₂-Cy-C(O)—. In some embodiments, L is —C(O)-Cy-CH₂-Cy-. In some embodiments, L is -Cy-CH₂-Cy-NR—. In some embodiments, L is —NR-Cy-CH₂-Cy-. In some embodiments, L is —OCH₂CH₂-Cy-CH₂-Cy-NR—. In some embodiments, L is —NR-Cy-CH₂-Cy-CH₂CH₂O—. In some embodiments, L is —NR-Cy-CH₂-Cy-C(O)CH₂—. In some embodiments, L is —NRCH₂CH₂NRC(O)CH₂-Cy-CH₂C(O)—. In some embodiments, L is —C(O)CH₂-Cy-CH₂C(O)NRCH₂CH₂NR—. In some embodiments, L is —C(O)-Cy-CH₂NRCH₂C(O)—. In some embodiments, L is —NR-Cy-CH₂NRCH₂C(O)NRCH₂CH₂NR—. In some embodiments, L is —NRCH₂CH₂NRC(O)CH₂NRCH₂-Cy-NR—.

In some embodiments, L is a covalent bond.

In some embodiments, L is —C(O)—CH₂-Cy-CH₂-Cy-. In some embodiments, L is —C(O)—CH₂-Cy-O-Cy-. In some embodiments, L is —C(O)—CH₂-Cy-. In some embodiments, wherein L is -Cy-CH₂-Cy-. In some embodiments, L is -Cy-O-Cy-.

Without limitation, the point of attachment of L to PBM and DIM can be, for example when L is

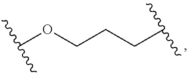 [SMILES: *CCCO*]

either

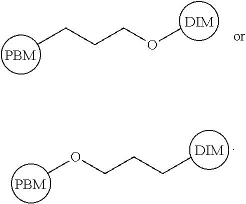

In some embodiments. L is

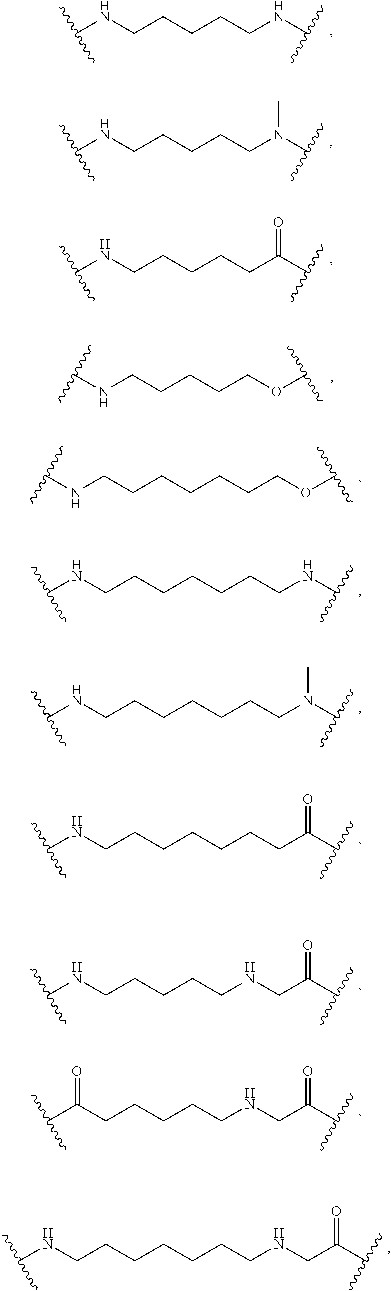

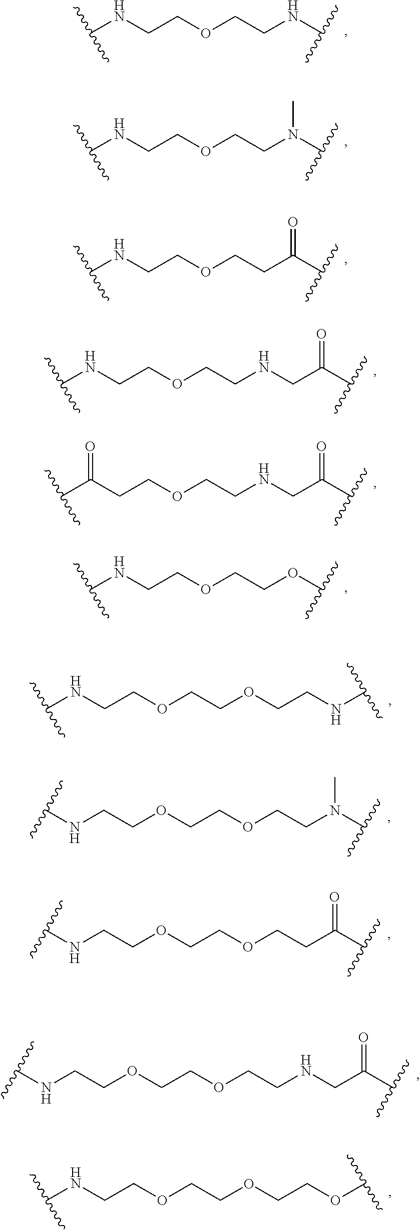

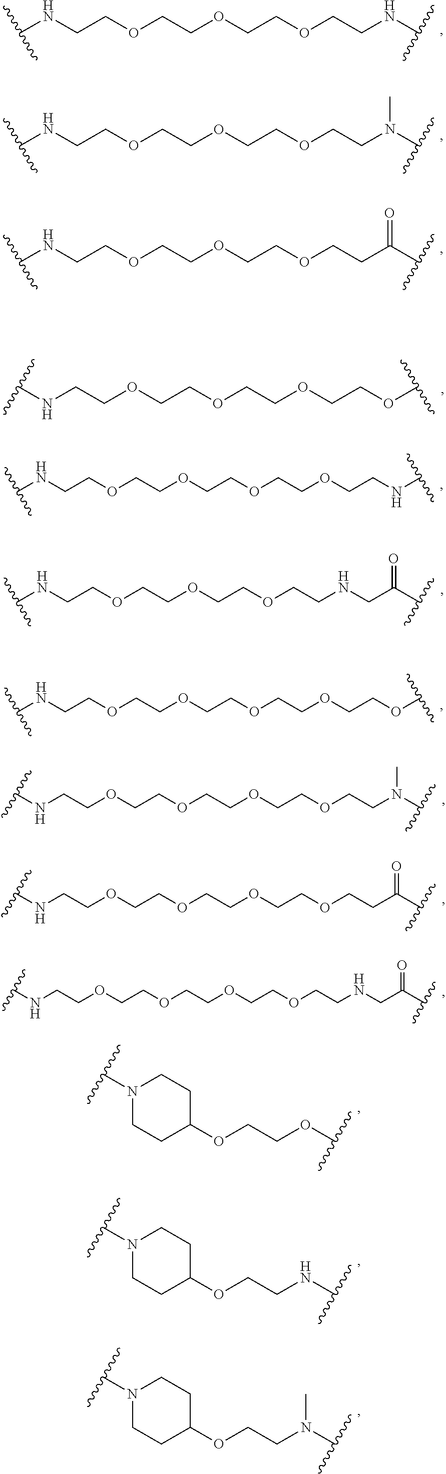

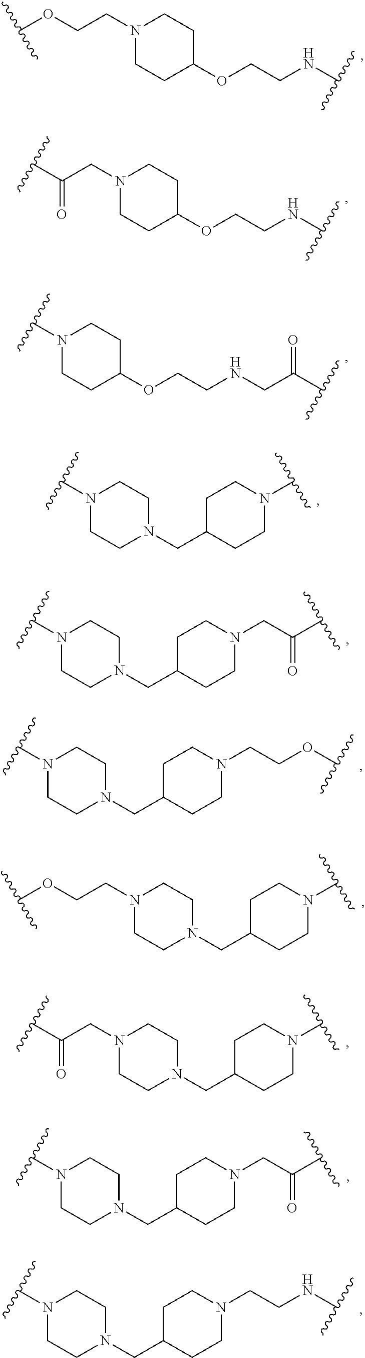

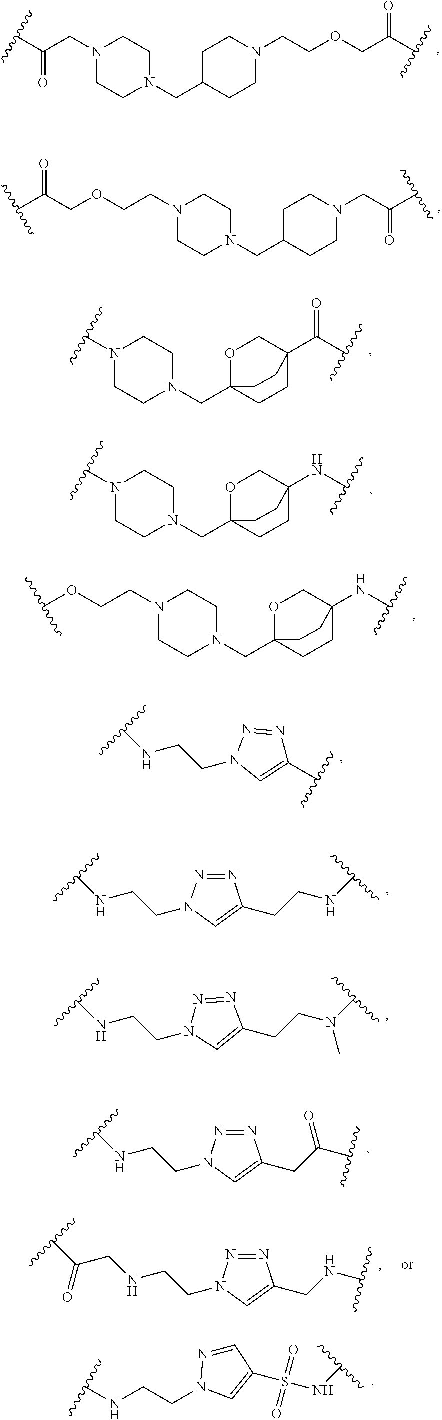

In some embodiments, L is

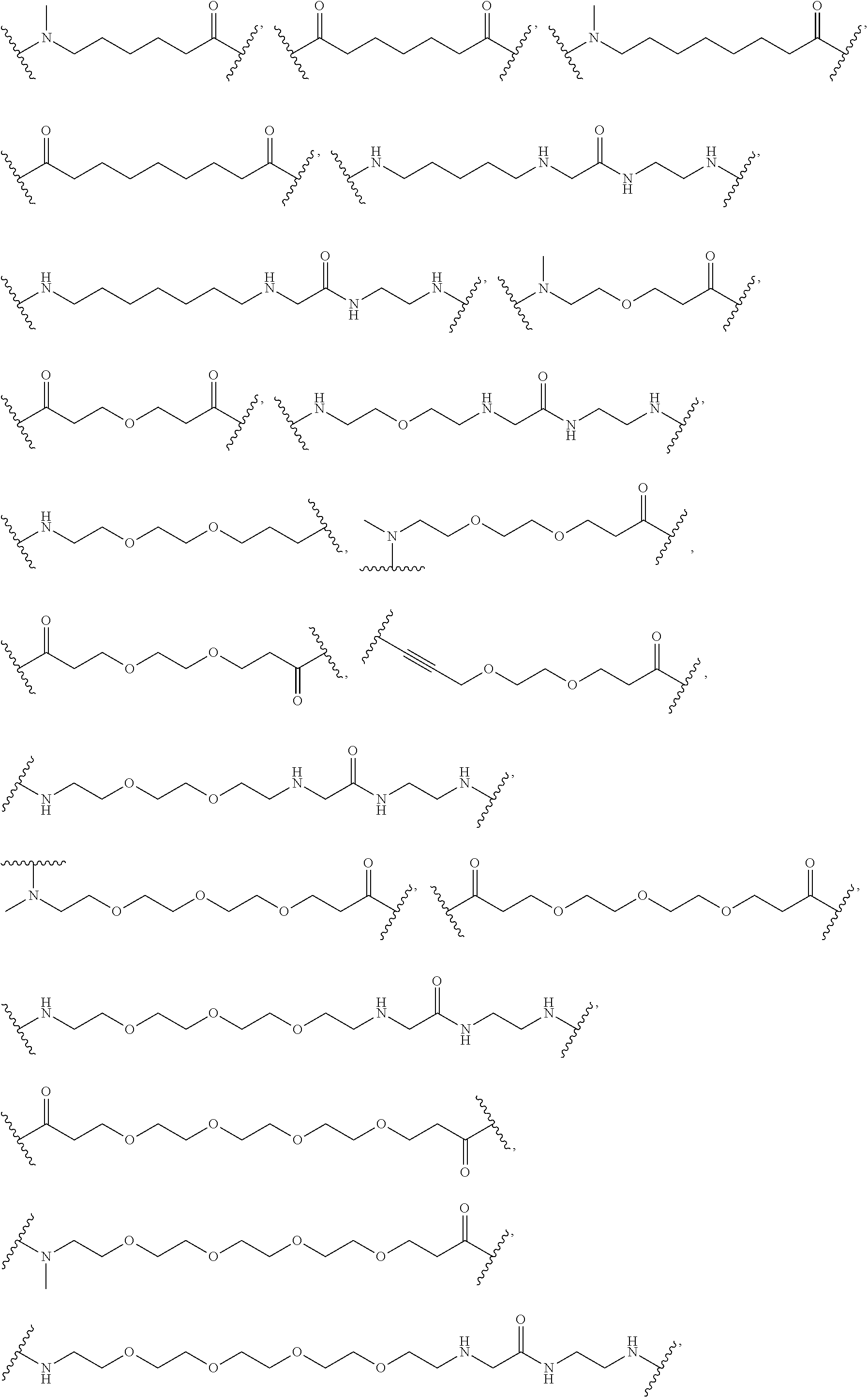

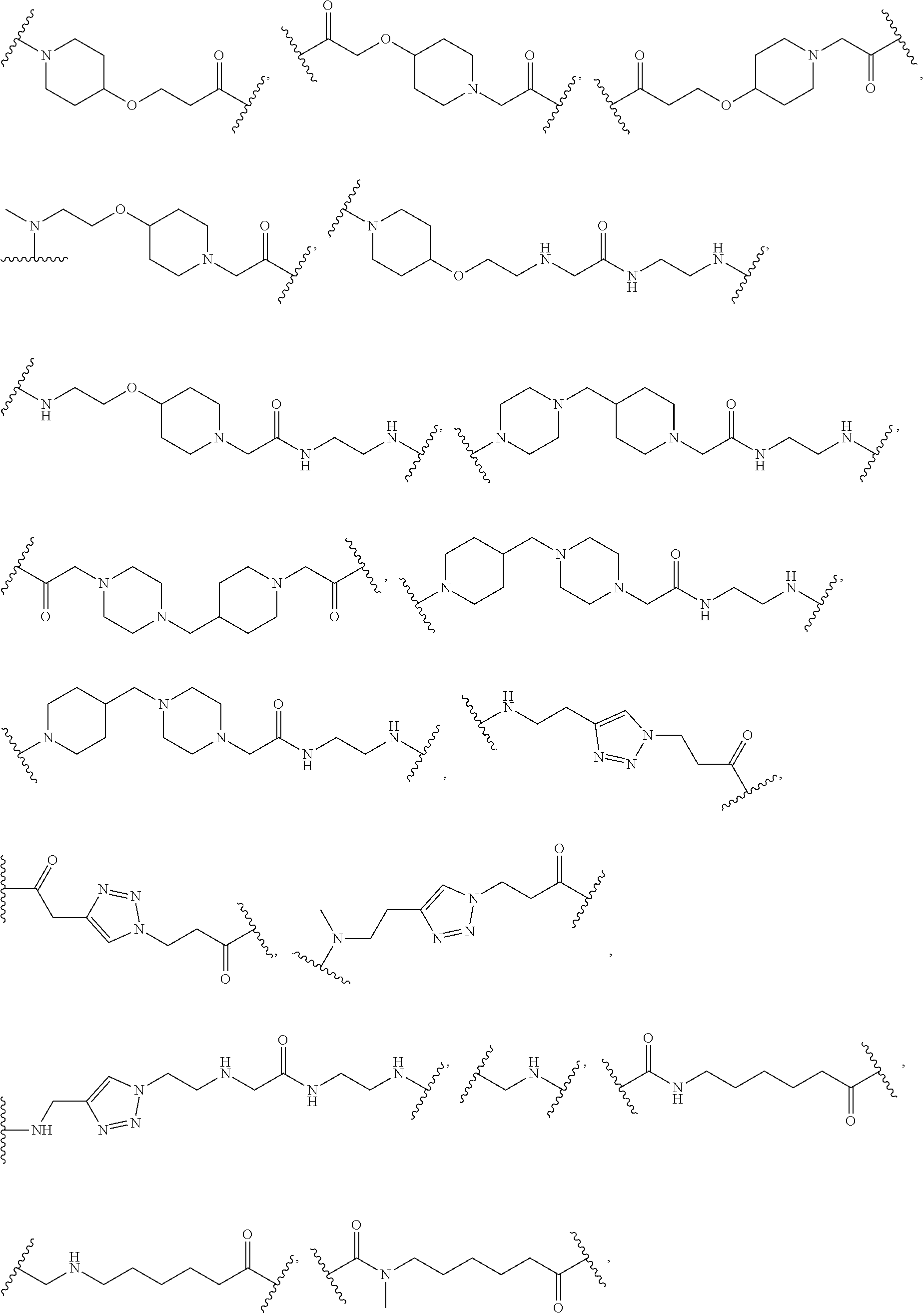

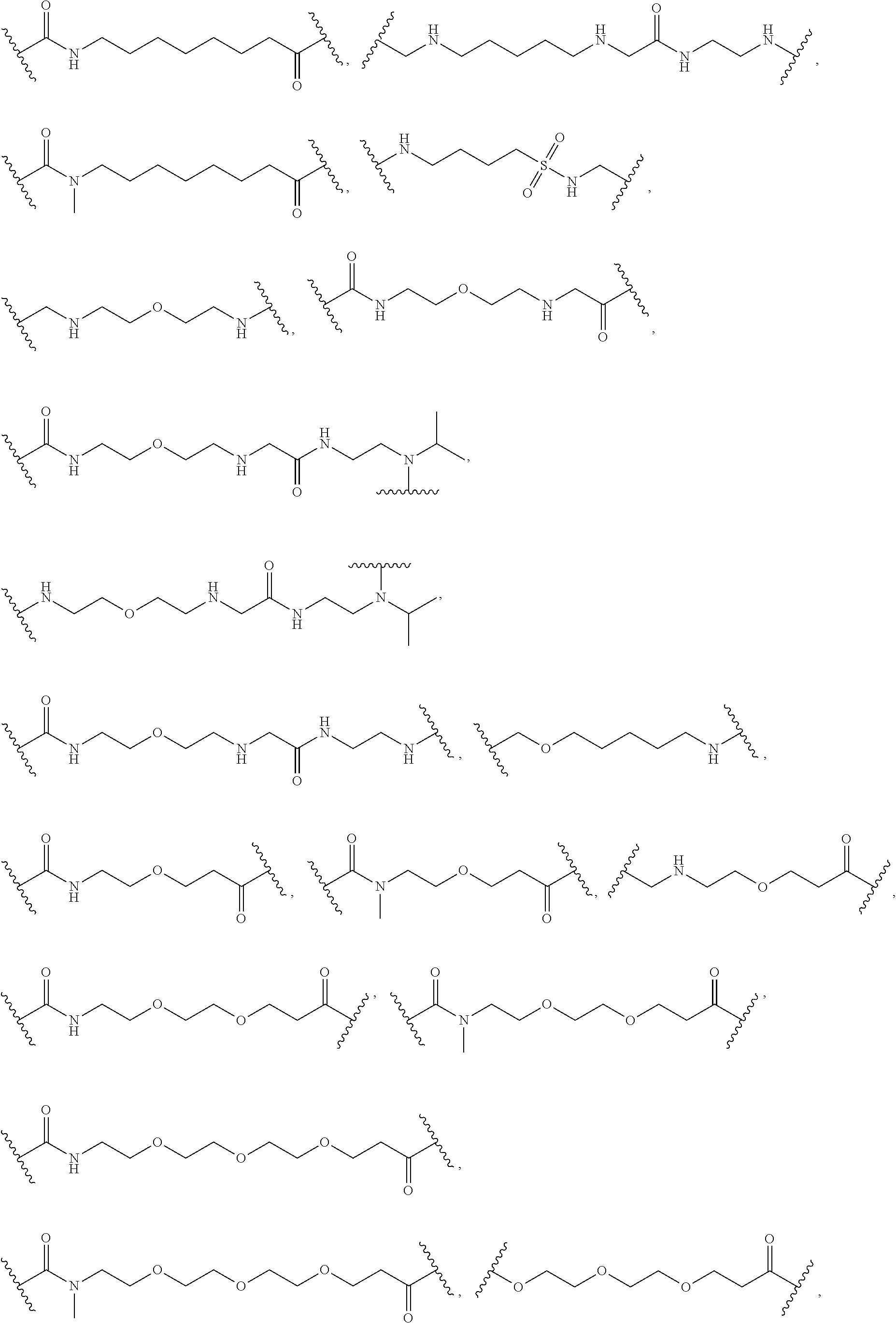

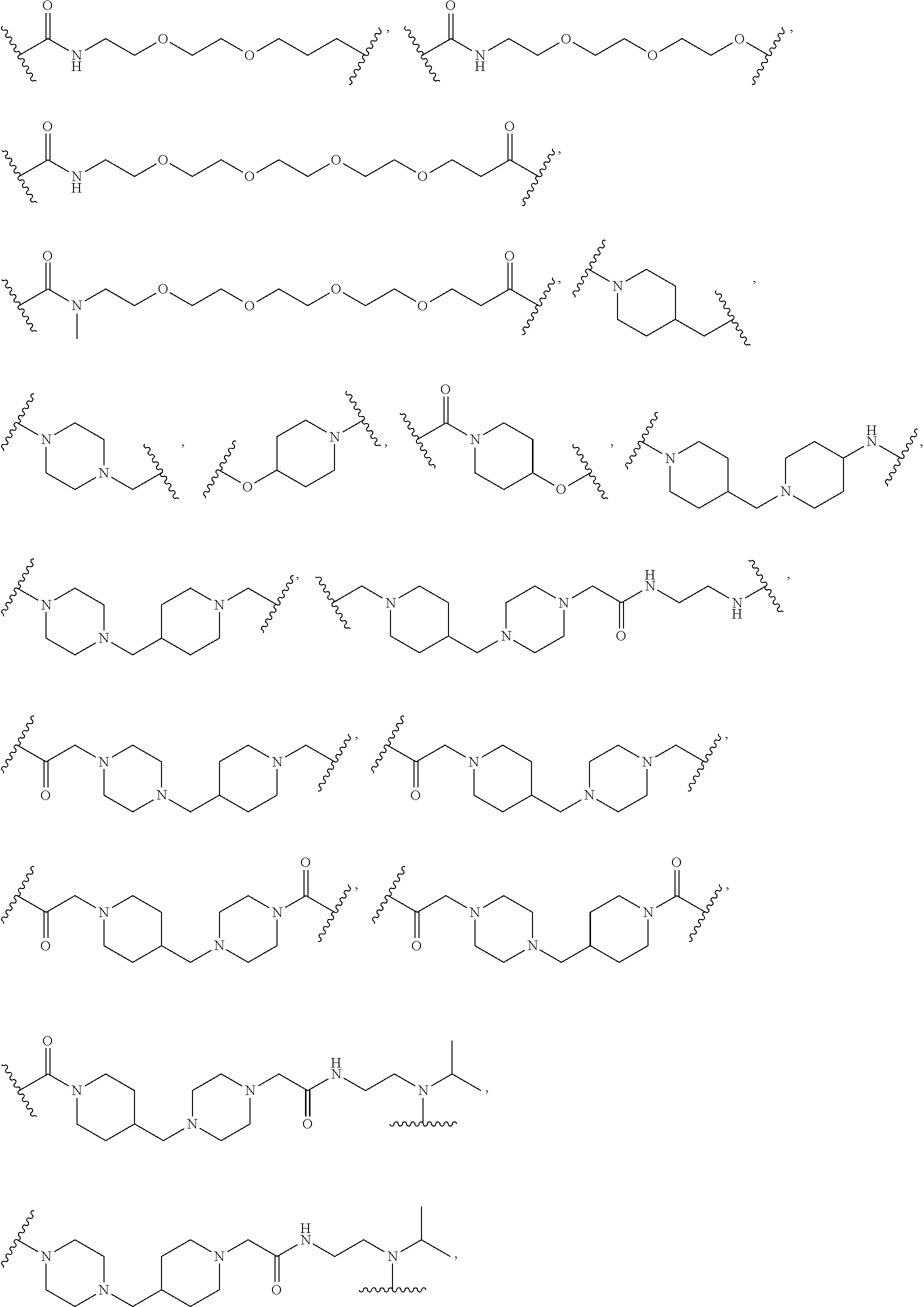

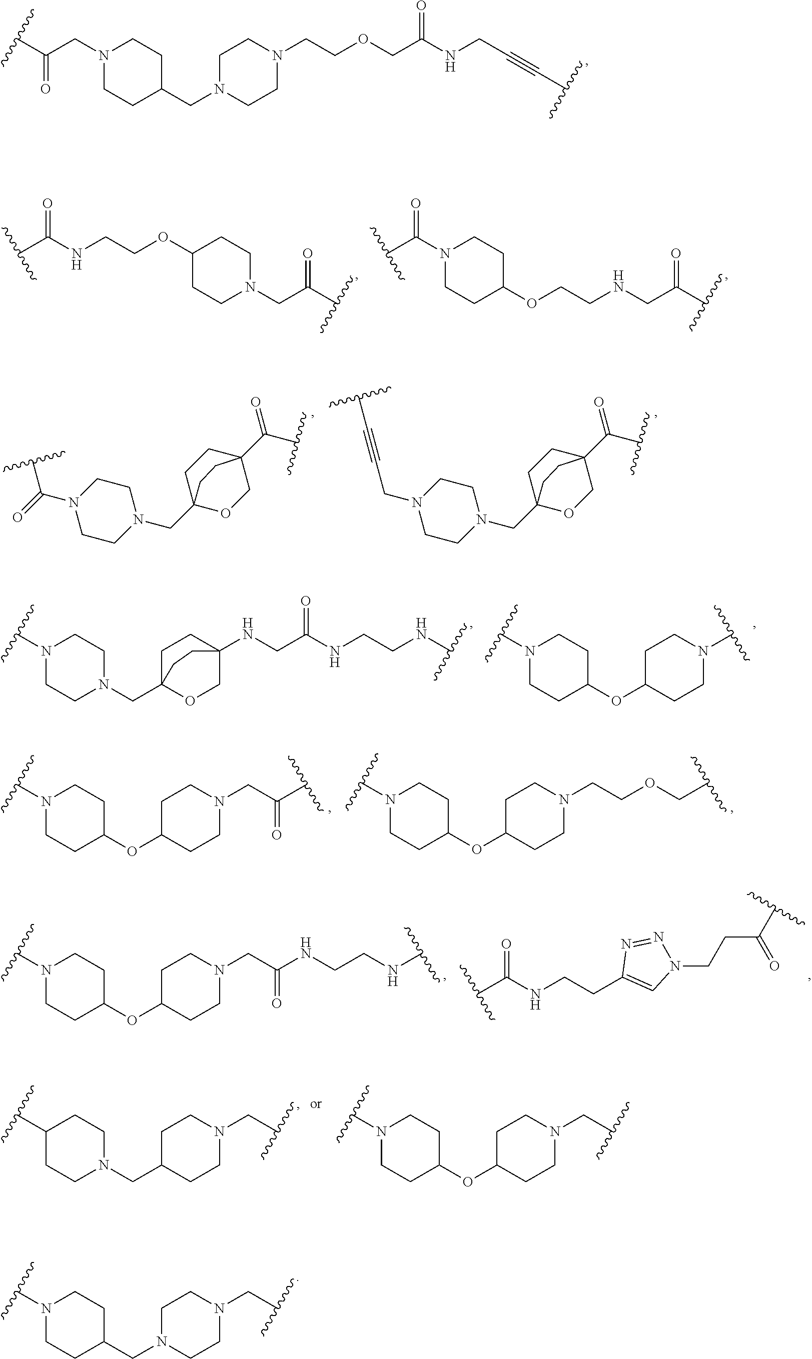

In some embodiments, L is

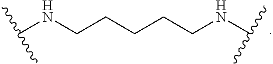 [SMILES: *NCCCCCN*]

In some embodiments, L is

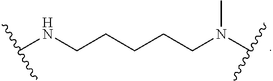 [SMILES: *NCCCCCN(*)C]

In some embodiments, L is

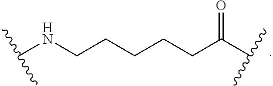 [SMILES: *NCCCCCC(*)=O]

In some embodiments, L is

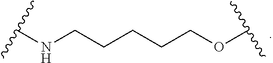 [SMILES: *NCCCCCO*]

In some embodiments, L is

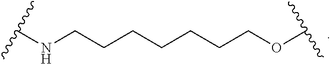 [SMILES: *NCCCCCCCO*]

In some embodiments, L is

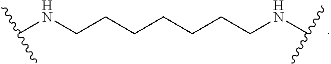 [SMILES: *NCCCCCCCN*]

In some embodiments, L is

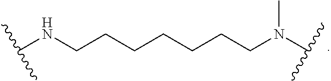 [SMILES: *NCCCCCCCN(*)C]

In some embodiments, L is

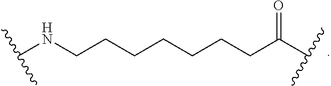 [SMILES: *NCCCCCCCC(*)=O]

In some embodiments, L is

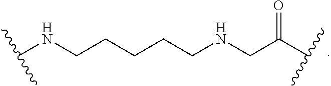 [SMILES: *NCCCCCNCC(*)=O]

In some embodiments, L is

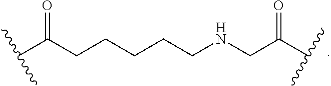 [SMILES: *C(=O)CCCCCNCC(*)=O]

In some embodiments, L is

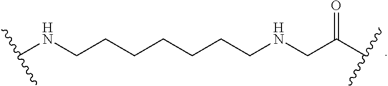 [SMILES: *NCCCCCCCNCC(*)=O]

In some embodiments, L is

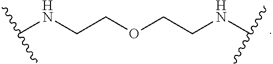 [SMILES: *NCCOCCN*]

In some embodiments, L is

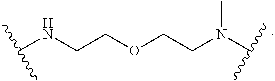 [SMILES: *NCCOCCN(*)C]

In some embodiments, L is

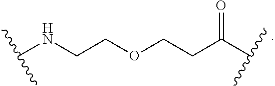 [SMILES: *NCCOCCC(*)=O]

In some embodiments, L is

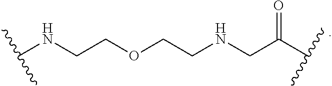 [SMILES: *NCCOCCNCC(*)=O]

In some embodiments, L is

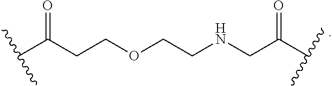 [SMILES: *C(=O)CCOCCNCC(*)=O]

In some embodiments, L is

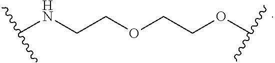 [SMILES: *NCCOCCO*]

In some embodiments, L is

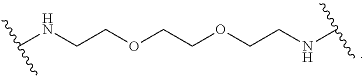 [SMILES: *NCCOCCOCCN*]

In some embodiments, L is

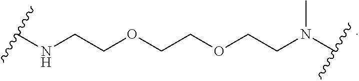 [SMILES: *NCCOCCOCCN(*)C]

In some embodiments, L is

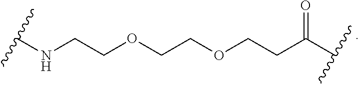 [SMILES: *NCCOCCOCCC(*)=O]

In some embodiments, L is

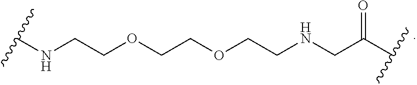 [SMILES: *NCCOCCOCCNCC(*)=O]

In some embodiments, L is

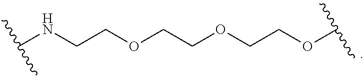 [SMILES: *NCCOCCOCCO*]

In some embodiments, L is

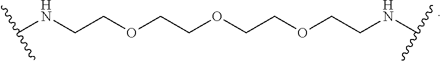 [SMILES: *NCCOCCOCCOCCN*]

In some embodiments, L is

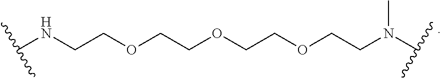 [SMILES: *NCCOCCOCCOCCN(*)C]

In some embodiments, L is

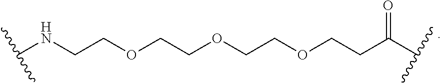 [SMILES: *NCCOCCOCCOCCC(*)=O]

In some embodiments, L is

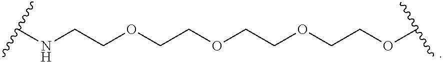 [SMILES: *NCCOCCOCCOCCO*]

In some embodiments, L is

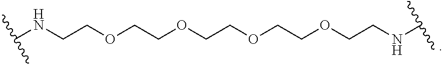 [SMILES: *NCCOCCOCCOCCOCCN*]

In some embodiments, L is

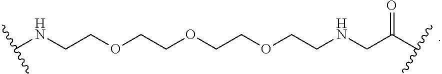 [SMILES: *NCCOCCOCCOCCNCC(*)=O]

In some embodiments, L is

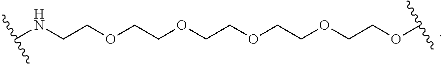 [SMILES: *NCCOCCOCCOCCOCCO*]

In some embodiments, L is

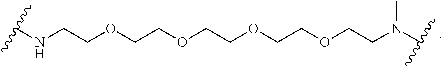 [SMILES: *NCCOCCOCCOCCOCCN(*)C]

In some embodiments, L is

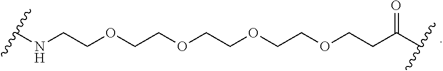 [SMILES: *NCCOCCOCCOCCOCCC(*)=O]

In some embodiments, L is

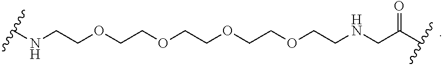 [SMILES: *NCCOCCOCCOCCOCCNCC(*)=O]

In some embodiments, L is

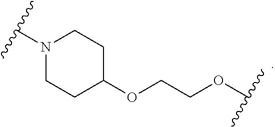 [SMILES: *OCCOC1CCN(*)CC1]

In some embodiments, L is

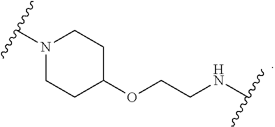 [SMILES: *NCCOC1CCN(*)CC1]

In some embodiments, L is

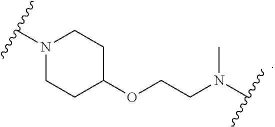 [SMILES: *N(C)CCOC1CCN(*)CC1]

In some embodiments, L is

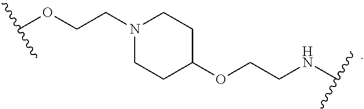 [SMILES: *NCCOC1CCN(CCO*)CC1]

In some embodiments, L is

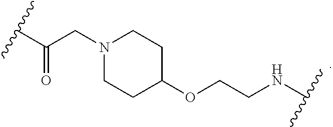 [SMILES: *NCCOC1CCN(CC(*)=O)CC1]

In some embodiments, L is

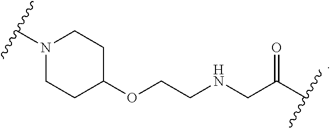 [SMILES: *C(=O)CNCCOC1CCN(*)CC1]

In some embodiments, L is

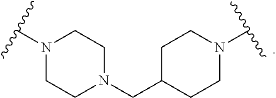 [SMILES: *N1CCC(CN2CCN(*)CC2)CC1]

In some embodiments, L is

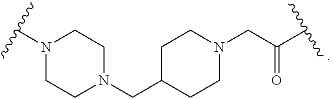 [SMILES: *C(=O)CN1CCC(CN2CCN(*)CC2)CC1 ; "."]

In some embodiments, L is

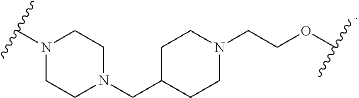 [SMILES: "." ; *OCCN1CCC(CN2CCN(*)CC2)CC1]

In some embodiments, L is

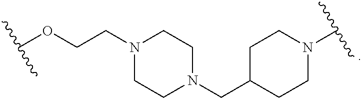 [SMILES: *OCCN1CCN(CC2CCN(*)CC2)CC1 ; "."]

In some embodiments, L is

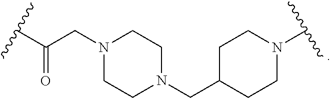 [SMILES: *C(=O)CN1CCN(CC2CCN(*)CC2)CC1 ; "."]

In some embodiments, L is

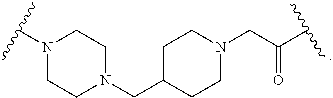 [SMILES: *C(=O)CN1CCC(CN2CCN(*)CC2)CC1 ; "."]

In some embodiments, L is

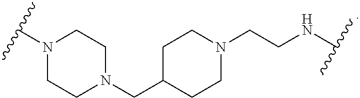 [SMILES: "." ; *NCCN1CCC(CN2CCN(*)CC2)CC1]

In some embodiments, L is

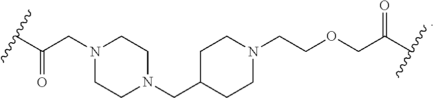 [SMILES: *C(=O)COCCN1CCC(CN2CCN(CC(*)=O)CC2)CC1 ; "."]

In some embodiments, L is

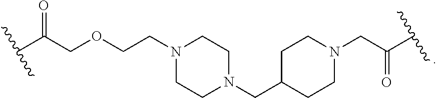 [SMILES: *C(=O)COCCN1CCN(CC2CCN(CC(*)=O)CC2)CC1 ; "."]

In some embodiments, L is

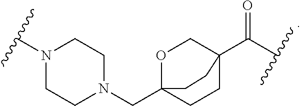 [SMILES: *C(=O)C12CCC(CN3CCN(*)CC3)(CC1)OC2 ; "."]

In some embodiments, L is

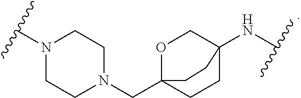 [SMILES: "." ; *NC12CCC(CN3CCN(*)CC3)(CC1)OC2]

In some embodiments, L is

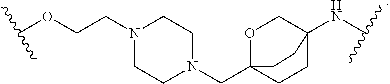 [SMILES: "." ; *NC12CCC(CN3CCN(CCO*)CC3)(CC1)OC2]

In some embodiments, L is

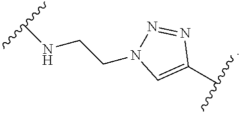 [SMILES: *NCCn1cc(*)nn1 ; "."]

In some embodiments, L is

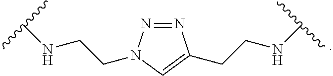 [SMILES: *NCCc1cn(CCN*)nn1 ; "."]

In some embodiments, L is

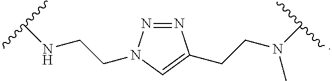 [SMILES: *NCCn1cc(CCN(*)C)nn1 ; "."]

In some embodiments, L is

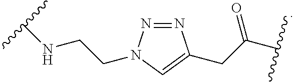 [SMILES: *NCCn1cc(CC(*)=O)nn1 ; "."]

In some embodiments, L is

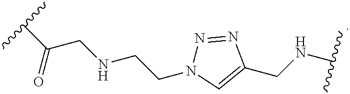 [SMILES: *NCc1cn(CCNCC(*)=O)nn1 ; "."]

In some embodiments, L is

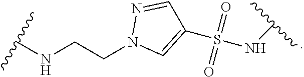 [SMILES: *NCCn1cc(S(=O)(=O)N*)cn1 ; "."]

In some embodiments, L is

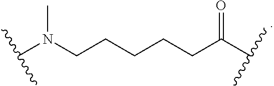 [SMILES: *C(=O)CCCCCN(*)C ; "."]

In some embodiments, L is

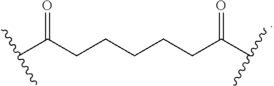 [SMILES: *C(=O)CCCCCC(*)=O ; "."]

In some embodiments, L is

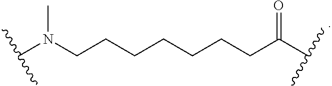 [SMILES: *C(=O)CCCCCCCN(*)C ; "."]

In some embodiments, L is

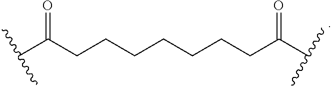 [SMILES: *C(=O)CCCCCCCC(*)=O ; "."]

In some embodiments, L is

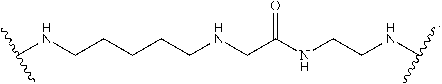 [SMILES: *NCCCCCNCC(=O)NCCN* ; "."]

In some embodiments, L is

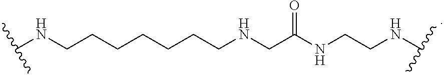 [SMILES: *NCCCCCCCNCC(=O)NCCN* ; "."]

In some embodiments, L is

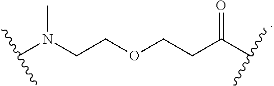 [SMILES: *C(=O)CCOCCN(*)C ; "."]

In some embodiments, L is

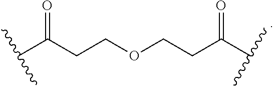 [SMILES: *C(=O)CCOCCC(*)=O ; "."]

In some embodiments, L is

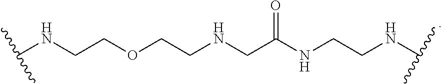 [SMILES: *NCCNC(=O)CNCCOCCN* ; "."]

In some embodiments, L is

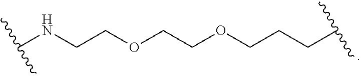 [SMILES: *CCCOCCOCCN* ; "."]

In some embodiments, L is

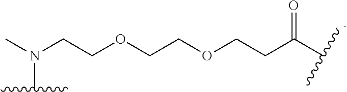 [SMILES: *C(=O)CCOCCOCCN(*)C ; "."]

In some embodiments, L is

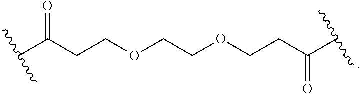 [SMILES: *C(=O)CCOCCOCCC(*)=O ; "."]

In some embodiments, L is

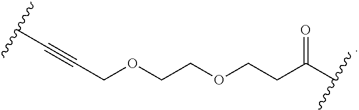 [SMILES: *C#CCOCCOCCC(*)=O ; "."]

In some embodiments, L is

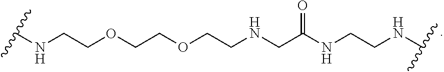 [SMILES: *NCCNC(=O)CNCCOCCOCCN*]

In some embodiments, L is

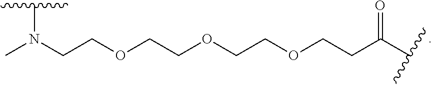 [SMILES: *C(=O)CCOCCOCCOCCN(*)C]

In some embodiments, L is

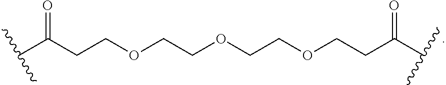 [SMILES: *C(=O)CCOCCOCCOCCC(*)=O]

In some embodiments, L is

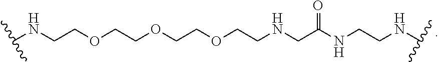 [SMILES: *NCCNC(=O)CNCCOCCOCCOCCN*]

In some embodiments, L is

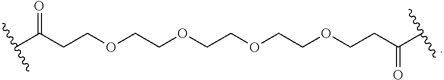 [SMILES: *C(=O)CCOCCOCCOCCOCCC(*)=O]

In some embodiments, L is

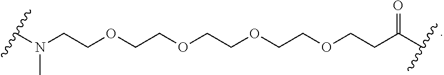 [SMILES: *C(=O)CCOCCOCCOCCOCCN(*)C]

In some embodiments, L is

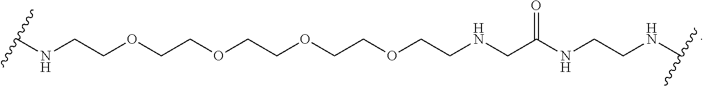 [SMILES: *NCCNC(=O)CNCCOCCOCCOCCOCCN*]

In some embodiments, L is

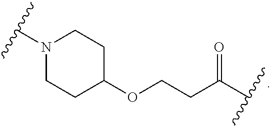 [SMILES: *C(=O)CCOC1CCN(*)CC1]

In some embodiments, L is

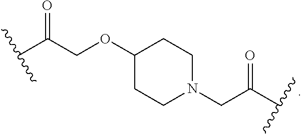 [SMILES: *C(=O)COC1CCN(CC(*)=O)CC1]

In some embodiments, L is

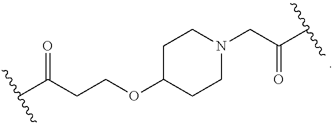 [SMILES: *C(=O)CCOC1CCN(CC(*)=O)CC1]

In some embodiments, L is

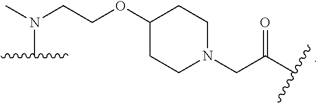 [SMILES: *C(=O)CN1CCC(OCCN(*)C)CC1]

In some embodiments, L is

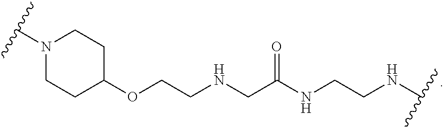 [SMILES: *NCCNC(=O)CNCCOC1CCN(*)CC1]

In some embodiments, L is

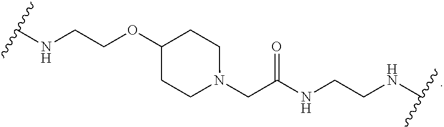 [SMILES: *NCCNC(=O)CN1CCC(OCCN*)CC1]

In some embodiments, L is

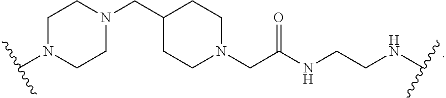 [SMILES: *NCCNC(=O)CN1CCC(CN2CCN(*)CC2)CC1]

In some embodiments, L is

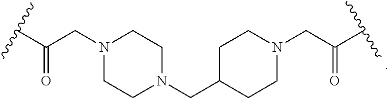 [SMILES: *C(=O)CN1CCC(CN2CCN(CC(*)=O)CC2)CC1]

In some embodiments, L is

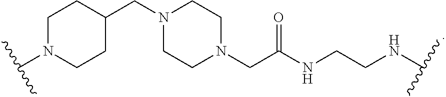 [SMILES: *NCCNC(=O)CN1CCN(CC2CCN(*)CC2)CC1 ; "."]

In some embodiments, L is

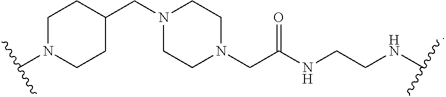 [SMILES: *NCCNC(=O)CN1CCN(CC2CCN(*)CC2)CC1 ; "."]

In some embodiments, L is

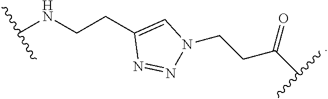 [SMILES: *NCCc1cn(CCC(*)=O)nn1 ; "."]

In some embodiments, L is

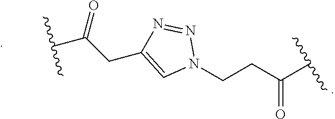 [SMILES: "." ; *C(=O)CCn1cc(CC(*)=O)nn1 ; "."]

In some embodiments, L is

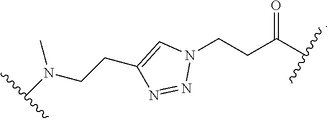 [SMILES: *C(=O)CCn1cc(CCN(*)C)nn1 ; "."]

In some embodiments, L is

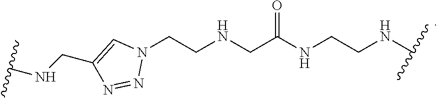 [SMILES: *NCCNC(=O)CNCCn1cc(CN*)nn1 ; "."]

In some embodiments, L is

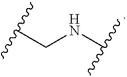 [SMILES: "." ; *CN*]

In some embodiments, L is

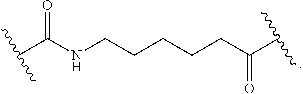 [SMILES: *C(=O)CCCCCNC(*)=O ; "."]

In some embodiments, L is

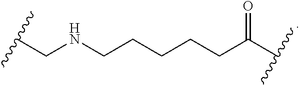 [SMILES: *CNCCCCCC(*)=O ; "."]

In some embodiments, L is

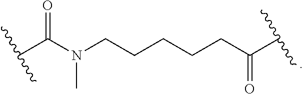 [SMILES: *C(=O)CCCCCN(C)C(*)=O ; "."]

In some embodiments, L is

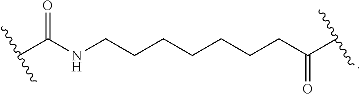 [SMILES: *C(=O)CCCCCCCNC(*)=O ; "."]

In some embodiments, L is

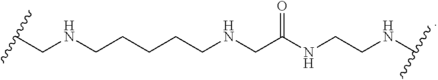 [SMILES: *CNCCCCCNCC(=O)NCCN* ; "."]

In some embodiments, L is

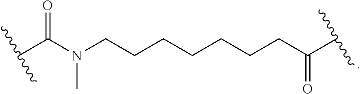 [SMILES: *C(=O)CCCCCCCN(C)C(*)=O ; "."]

In some embodiments, L is

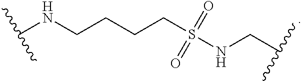 [SMILES: *CNS(=O)(=O)CCCCN* ; "."]

In some embodiments, L is

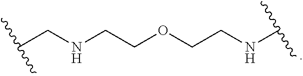 [SMILES: *CNCCOCCN* ; "."]

In some embodiments, L is

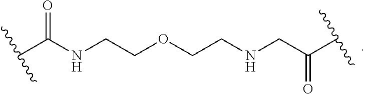 [SMILES: *C(=O)CNCCOCCNC(*)=O]

In some embodiments, L is

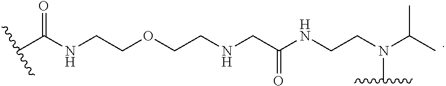 [SMILES: *C(=O)NCCOCCNCC(=O)NCCN(*)C(C)C]

In some embodiments, L is

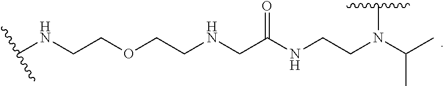 [SMILES: *NCCOCCNCC(=O)NCCN(*)C(C)C]

In some embodiments, L is

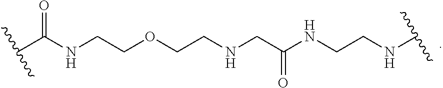 [SMILES: *NCCNC(=O)CNCCOCCNC(*)=O]

In some embodiments, L is

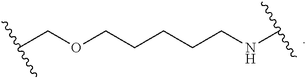 [SMILES: *COCCCCCN*]

In some embodiments, L is

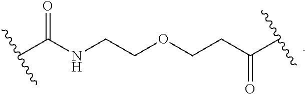 [SMILES: *C(=O)CCOCCNC(*)=O]

In some embodiments, L is

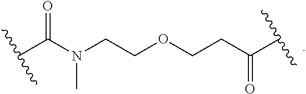 [SMILES: *C(=O)CCOCCN(C)C(*)=O]

In some embodiments, L is

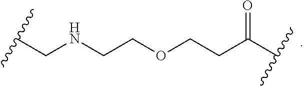 [SMILES: *CNCCOCCC(*)=O]

In some embodiments, L is

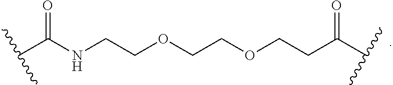 [SMILES: *C(=O)CCOCCOCCNC(*)=O]

In some embodiments, L is

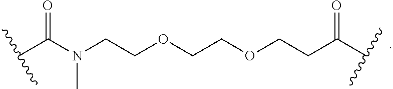 [SMILES: *C(=O)CCOCCOCCN(C)C(*)=O]

In some embodiments, L is

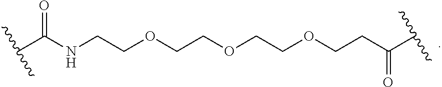 [SMILES: *C(=O)CCOCCOCCOCCNC(*)=O]

In some embodiments, L is

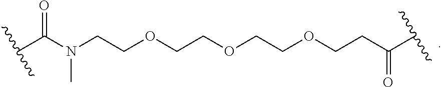 [SMILES: *C(=O)CCOCCOCCOCCN(C)C(*)=O]

In some embodiments, L is

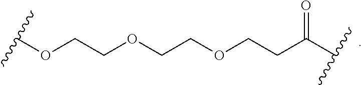 [SMILES: *OCCOCCOCCC(*)=O]

In some embodiments, L is

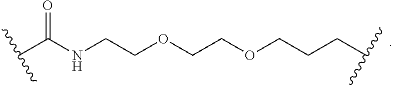 [SMILES: *CCCOCCOCCNC(*)=O]

In some embodiments, L is

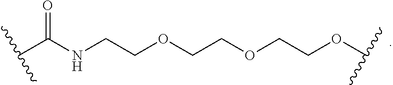 [SMILES: *OCCOCCOCCNC(*)=O]

In some embodiments, L is

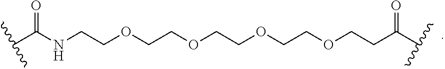 [SMILES: *C(=O)CCOCCOCCOCCOCCNC(*)=O]

In some embodiments, L is

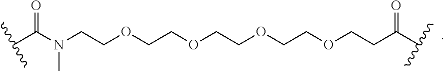 [SMILES: *C(=O)CCOCCOCCOCCOCCN(C)C(*)=O]

In some embodiments, L is

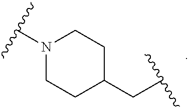 [SMILES: *CC1CCN(*)CC1]

In some embodiments, L is

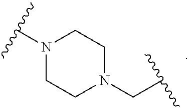 [SMILES: *CN1CCN(*)CC1]

In some embodiments, L is

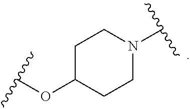 [SMILES: *OC1CCN(*)CC1]

In some embodiments, L is

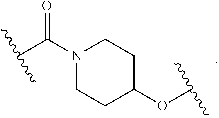 [SMILES: *OC1CCN(C(*)=O)CC1]

In some embodiments, L is

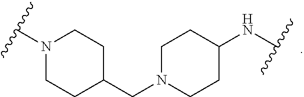 [SMILES: *NC1CCN(CC2CCN(*)CC2)CC1]

In some embodiments, L is

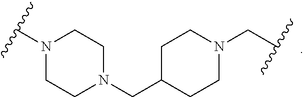 [SMILES: *CN1CCC(CN2CCN(*)CC2)CC1]

In some embodiments, L is

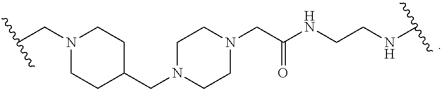 [SMILES: *CN1CCC(CN2CCN(CC(=O)NCCN*)CC2)CC1]

In some embodiments, L is

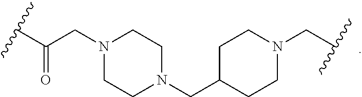 [SMILES: *CN1CCC(CN2CCN(CC(*)=O)CC2)CC1]

In some embodiments, L is

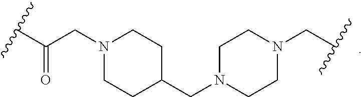 [SMILES: *CN1CCN(CC2CCN(CC(*)=O)CC2)CC1]

In some embodiments, L is

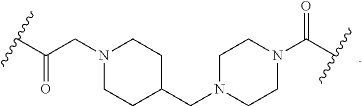 [SMILES: *C(=O)CN1CCC(CN2CCN(C(*)=O)CC2)CC1]

In some embodiments, L is

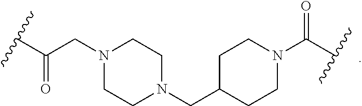 [SMILES: *C(=O)CN1CCN(CC2CCN(C(*)=O)CC2)CC1]

In some embodiments, L is

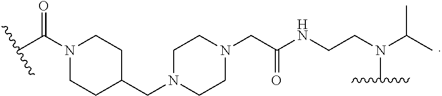 [SMILES: *C(=O)N1CCC(CN2CCN(CC(=O)NCCN(*)C(C)C)CC2)CC1]

In some embodiments, L is

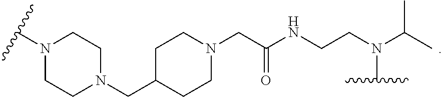 [SMILES: *N1CCN(CC2CCN(CC(=O)NCCN(*)C(C)C)CC2)CC1]

In some embodiments, L is

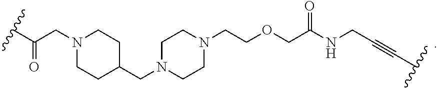 [SMILES: *C#CCNC(=O)COCCN1CCN(CC2CCN(CC(*)=O)CC2)CC1]

In some embodiments, L is

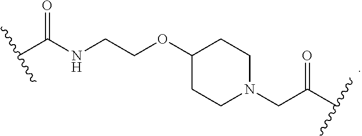 [SMILES: *C(=O)CN1CCC(OCCNC(*)=O)CC1]

In some embodiments, L is

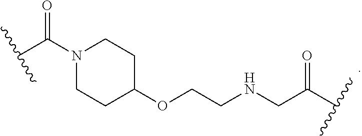 [SMILES: *C(=O)CNCCOC1CCN(C(*)=O)CC1]

In some embodiments, L is

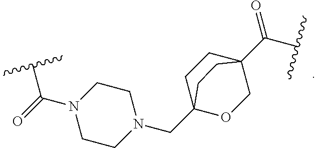 [SMILES: *C(=O)N1CCN(CC23CCC(C(*)=O)(CC2)CO3)CC1]

In some embodiments, L is

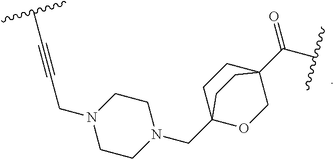 [SMILES: *C#CCN1CCN(CC23CCC(C(*)=O)(CC2)CO3)CC1]

In some embodiments, L is

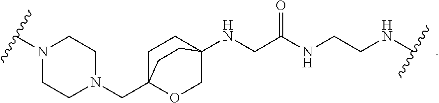 [SMILES: *NCCNC(=O)CNC12CCC(CN3CCN(*)CC3)(CC1)OC2]

In some embodiments, L is

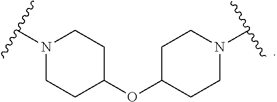 [SMILES: *N1CCC(OC2CCN(*)CC2)CC1]

In some embodiments, L is

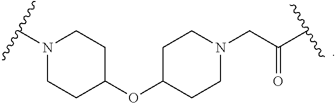 [SMILES: *C(=O)CN1CCC(OC2CCN(*)CC2)CC1]

In some embodiments, L is

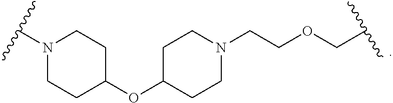 [SMILES: *COCCN1CCC(OC2CCN(*)CC2)CC1]

In some embodiments, L is

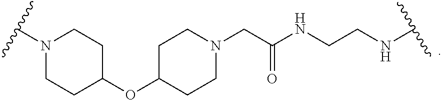 [SMILES: *NCCNC(=O)CN1CCC(OC2CCN(*)CC2)CC1]

In some embodiments, L is

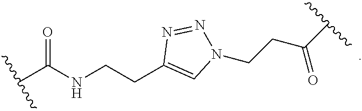 [SMILES: *C(=O)CCn1cc(CCNC(*)=O)nn1]

In some embodiments, L is

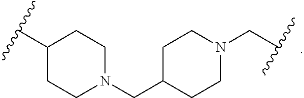 [SMILES: *CN1CCC(CN2CCC(*)CC2)CC1]

In some embodiments, L is

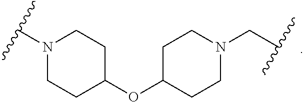 [SMILES: *CN1CCC(OC2CCN(*)CC2)CC1]

In some embodiments, L is

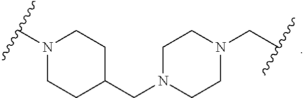 [SMILES: *CN1CCN(CC2CCN(*)CC2)CC1]

In some embodiments, L is selected from those depicted in Table 1, below.

In some embodiments, L is selected from those depicted in Table B, below.

In some embodiments, the present invention provides the compound of formula I-a, wherein DBM is

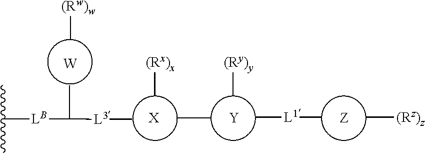

from formula I-bb, to provide a compound of formula I-abb:

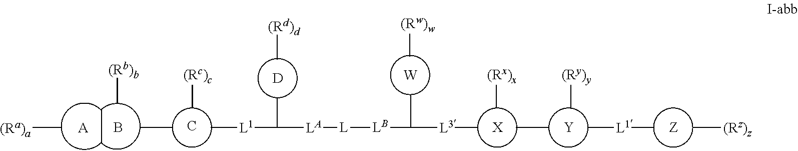

or a pharmaceutically acceptable salt thereof, wherein each of ring A, ring B, ring C, ring, D, L¹, L^{A}, R^{a}, R^{b}, R^{c}, R^{d}, a, b, c, d, L, ring W, ring X, ring Y, ring Z, L^{B}, L^{3′}, L^{2′}, R^{1′}, R^{x}, R^{y}, R^{z}, w, x, y, z, is independently as defined above and described in embodiments herein, both singly and in combination.

In some embodiments, the present invention provides the compound of formula I-a, wherein DBM is

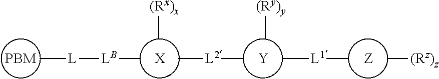

from formula I-cc, to provide a compound of formula I-acc:

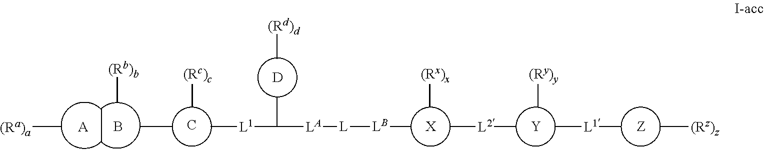

or a pharmaceutically acceptable salt thereof, wherein each of ring A, ring B, ring C, ring, D, L¹, L^{A}, R^{a}, R^{b}, R^{c}, R^{d}, a, b, c, d, L, ring X, ring Y, ring Z, L^{B}, L^{2′}, L^{1′}, R^{x}, R^{y}, R^{z}, x, y, z, is independently as defined above and described in embodiments herein, both singly and in combination.

In some embodiments, the present invention provides the compound of formula I-a, wherein DBM is

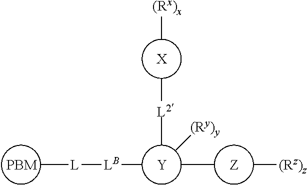

from formula I-ff, to provide a compound of formula I-aff:

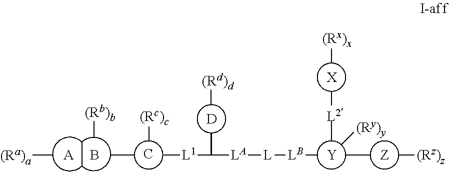

or a pharmaceutically acceptable salt thereof, wherein each of ring A, ring B, ring C, ring, D, L¹, L^{A}, R^{a}, R^{b}, R^{c}, R^{d}, a, b, c, d, L, ring X, ring Y, ring Z, L^{B}, L^{2′}, R^{x}, R^{y}, R^{z}, x, y, z, is independently as defined above and described in embodiments herein, both singly and in combination.

In some embodiments, the present invention provides the compound of formula I-a, wherein DBM is

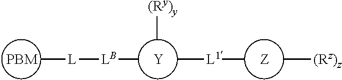

from formula I-gg, to provide a compound of formula I-agg:

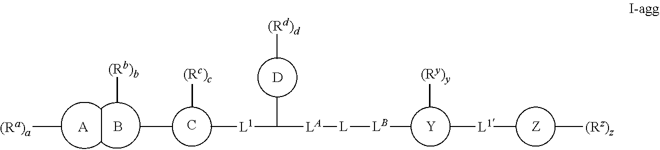

or a pharmaceutically acceptable salt thereof, wherein each of ring A, ring B, ring C, ring, D, L¹, L^{A}, R^{a}, R^{b}, R^{c}, R^{d}, a, b, c, d, L, ring Y, ring Z, L^{B}, L^{1′}, R^{y}, R^{z}, y, z, is independently as defined above and described in embodiments herein, both singly and in combination.

In some embodiments, the present invention provides the compound of formula I-a, wherein DBM is

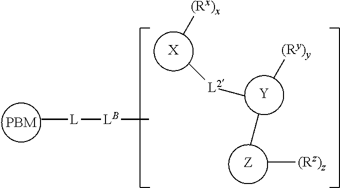

from formula I-hh, to provide a compound of formula I-ahh:

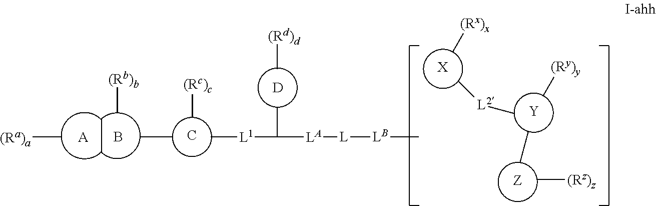

or a pharmaceutically acceptable salt thereof, wherein each of ring A, ring B, ring C, ring, D, L¹, L^{A}, R^{c}, R^{b}, R^{c}, R^{d}, a, b, c, d, L, ring X, ring Y, ring Z, L^{B}, L^{2′}, R^{x}, R^{y}, R^{z}, x, y, z, is independently as defined above and described in embodiments herein, both singly and in combination.

In some embodiments, the present invention provides the compound of formula I-a, wherein DBM is

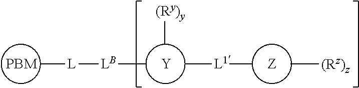

from formula I-ii, to provide a compound of formula I-aii:

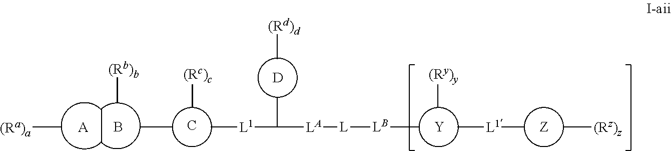

or a pharmaceutically acceptable salt thereof, wherein each of ring A, ring B, ring C, ring, D, L¹, L^{A}, R^{a}, R^{b}, R^{c}, R^{d}, a, b, c, d, L, ring Y, ring Z, L^{B}, L^{1′}, R^{y}, R^{z}, y, z, is independently as defined above and described in embodiments herein, both singly and in combination.

In some embodiments, the present invention provides the compound of formula I-a, wherein

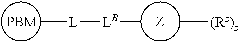

DBM is from formula I-kk, to provide a compound of formula I-akk:

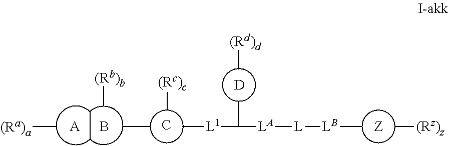

or a pharmaceutically acceptable salt thereof, wherein each of ring A, ring B, ring C, ring, D, L¹, L^{A}, R^{a}, R^{b}, R^{c}, R^{d}, a, b, c, d, L, ring Z, L^{B}, R^{z}, z, is independently as defined above and described in embodiments herein, both singly and in combination.

In some embodiments, the present invention provides the compound of formula I-a, wherein DBM is

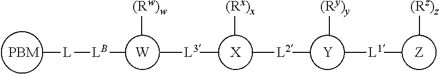

from formula I-ll, to provide a compound of formula I-all:

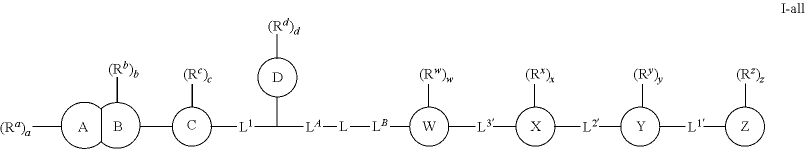

or a pharmaceutically acceptable salt thereof, wherein each of ring A, ring B, ring C, ring, D, L¹, L^{A}, R^{a}, R^{b}, R^{c}, R^{d}, a, b, c, d, L, ring W, ring X, ring Y, ring Z, L^{B}, L^{3′}, L^{2′}, L^{1′}, R^{w}, R^{x}, R^{y}, R^{z}, w, x, y, z, is independently as defined above and described in embodiments herein, both singly and in combination.

TABLE A
Exemplified Deubiquitinase binding moiety (DBM)

| | |
|---|---|
| 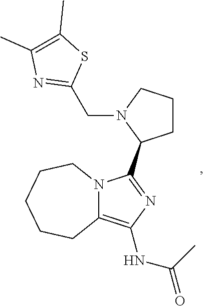 [SMILES: CC(=O)Nc1nc([C@@H]2CCCN2Cc2nc(C)c(C)s2)n2c1CCCCC2] | (a) |
| 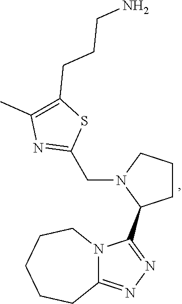 [SMILES: Cc1nc(CN2CCC[C@H]2c2nnc3n2CCCCC3)sc1CCCN] | (b) |
| 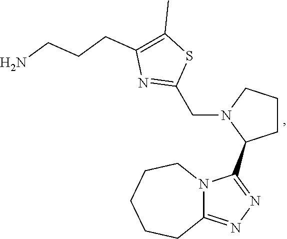 [SMILES: Cc1sc(CN2CCC[C@H]2c2nnc3n2CCCCC3)nc1CCCN] | (c) |
| 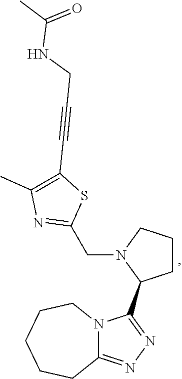 [SMILES: CC(=O)NCC#Cc1sc(CN2CCC[C@H]2c2nnc3n2CCCCC3)nc1C] | (d) |
| 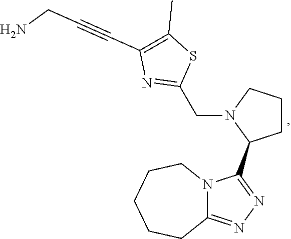 [SMILES: Cc1sc(CN2CCC[C@H]2c2nnc3n2CCCCC3)nc1C#CCN] | (e) |
| 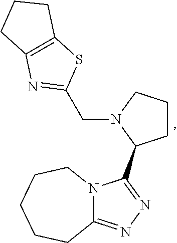 [SMILES: C1CCc2nnc([C@@H]3CCCN3Cc3nc4c(s3)CCC4)n2CC1] | (f) |
| 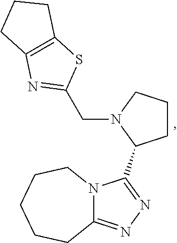 [SMILES: C1CCc2nnc([C@H]3CCCN3Cc3nc4c(s3)CCC4)n2CC1] | (g) |
| 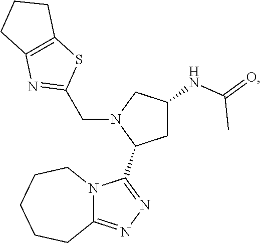 [SMILES: CC(=O)N[C@@H]1C[C@H](c2nnc3n2CCCCC3)N(Cc2nc3c(s2)CCC3)C1] | (h) |
| 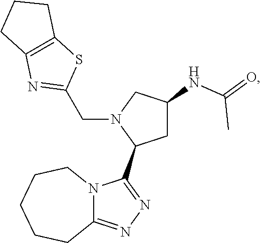 [SMILES: CC(=O)N[C@H]1C[C@@H](c2nnc3n2CCCCC3)N(Cc2nc3c(s2)CCC3)C1] | (i) |
| 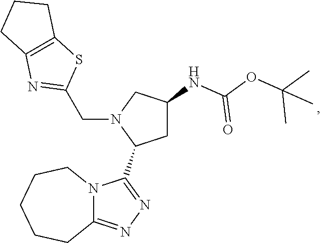 [SMILES: CC(C)(C)OC(=O)N[C@H]1C[C@H](c2nnc3n2CCCCC3)N(Cc2nc3c(s2)CCC3)C1] | (j) |
| 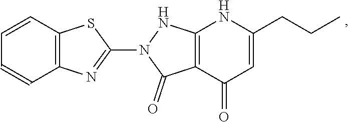 [SMILES: CCCc1cc(=O)c2c(=O)n(-c3nc4ccccc4s3)[nH]c2[nH]1] | (n) |
| 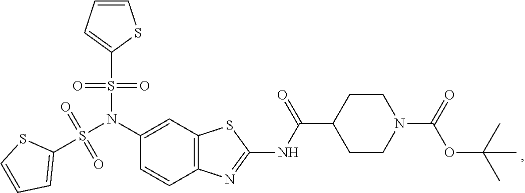 [SMILES: CC(C)(C)OC(=O)N1CCC(C(=O)Nc2nc3ccc(N(S(=O)(=O)c4cccs4)S(=O)(=O)c4cccs4)cc3s2)CC1] | (o) |
| 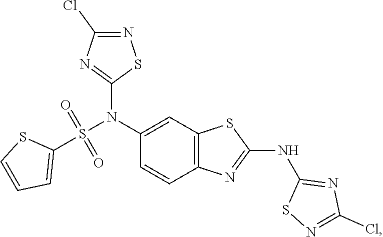 [SMILES: O=S(=O)(c1cccs1)N(c1ccc2nc(Nc3nc(Cl)ns3)sc2c1)c1nc(Cl)ns1] | (p) |
| 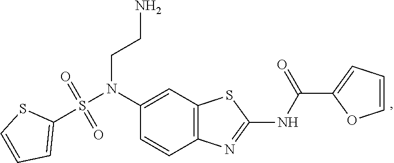 [SMILES: NCCN(c1ccc2nc(NC(=O)c3ccco3)sc2c1)S(=O)(=O)c1cccs1] | (q) |
| 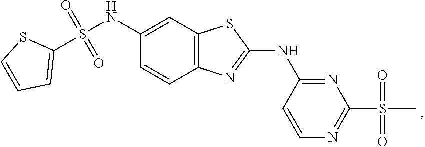 [SMILES: CS(=O)(=O)c1nccc(Nc2nc3ccc(NS(=O)(=O)c4cccs4)cc3s2)n1] | (r) |
| 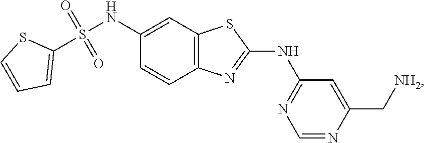 [SMILES: NCc1cc(Nc2nc3ccc(NS(=O)(=O)c4cccs4)cc3s2)ncn1] | (s) |
| 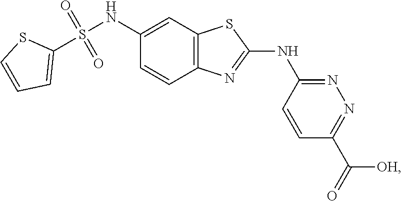 [SMILES: O=C(O)c1ccc(Nc2nc3ccc(NS(=O)(=O)c4cccs4)cc3s2)nn1] | (t) |
| 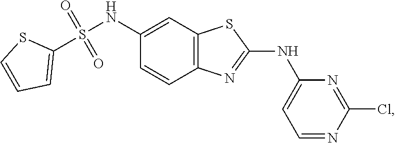 [SMILES: O=S(=O)(Nc1ccc2nc(Nc3ccnc(Cl)n3)sc2c1)c1cccs1] | (u) |
| 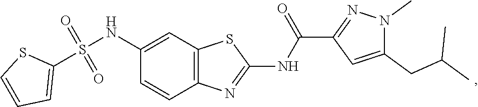 [SMILES: CC(C)Cc1cc(C(=O)Nc2nc3ccc(NS(=O)(=O)c4cccs4)cc3s2)nn1C] | (v) |
| 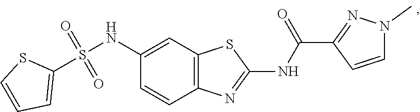 [SMILES: Cn1ccc(C(=O)Nc2nc3ccc(NS(=O)(=O)c4cccs4)cc3s2)n1] | (w) |
| 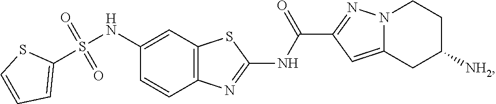 [SMILES: N[C@H]1CCn2nc(C(=O)Nc3nc4ccc(NS(=O)(=O)c5cccs5)cc4s3)cc2C1] | (x) |
| 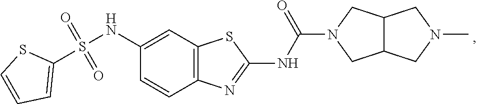 [SMILES: CN1CC2CN(C(=O)Nc3nc4ccc(NS(=O)(=O)c5cccs5)cc4s3)CC2C1] | (y) |
| 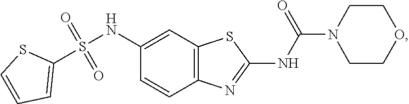 [SMILES: O=C(Nc1nc2ccc(NS(=O)(=O)c3cccs3)cc2s1)N1CCOCC1] | (z) |
| 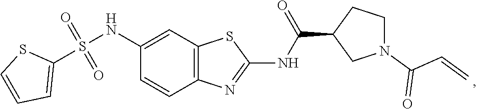 [SMILES: C=CC(=O)N1CC[C@H](C(=O)Nc2nc3ccc(NS(=O)(=O)c4cccs4)cc3s2)C1] | (aa) |
| 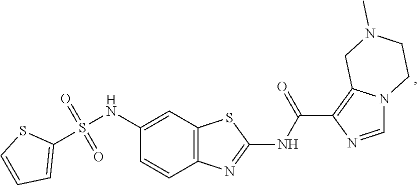 [SMILES: CN1CCn2cnc(C(=O)Nc3nc4ccc(NS(=O)(=O)c5cccs5)cc4s3)c2C1] | (ab) |
| 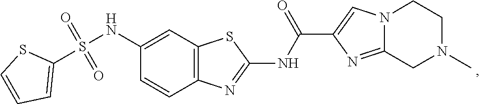 [SMILES: CN1CCn2cc(C(=O)Nc3nc4ccc(NS(=O)(=O)c5cccs5)cc4s3)nc2C1] | (ac) |
| 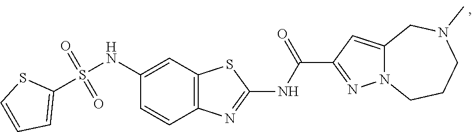 [SMILES: CN1CCCn2nc(C(=O)Nc3nc4ccc(NS(=O)(=O)c5cccs5)cc4s3)cc2C1] | (ad) |
| 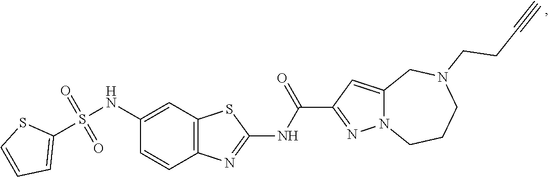 [SMILES: C#CCCN1CCCn2nc(C(=O)Nc3nc4ccc(NS(=O)(=O)c5cccs5)cc4s3)cc2C1] | (ae) |
| 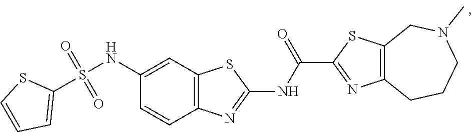 [SMILES: CN1CCCc2nc(C(=O)Nc3nc4ccc(NS(=O)(=O)c5cccs5)cc4s3)sc2C1] | (af) |
| 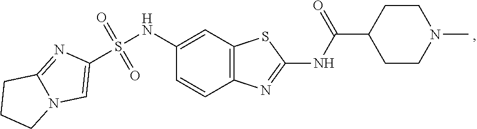 [SMILES: CN1CCC(C(=O)Nc2nc3ccc(NS(=O)(=O)c4cn5c(n4)CCC5)cc3s2)CC1] | (ag) |
| 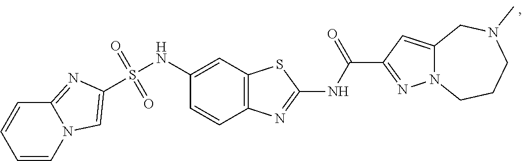 [SMILES: CN1CCCn2nc(C(=O)Nc3nc4ccc(NS(=O)(=O)c5cn6ccccc6n5)cc4s3)cc2C1] | (ah) |
| 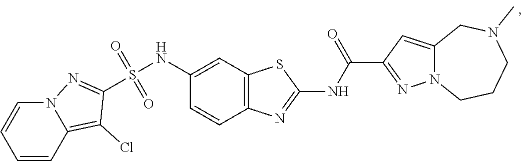 [SMILES: CN1CCCn2nc(C(=O)Nc3nc4ccc(NS(=O)(=O)c5nn6ccccc6c5Cl)cc4s3)cc2C1] | (ai) |
| 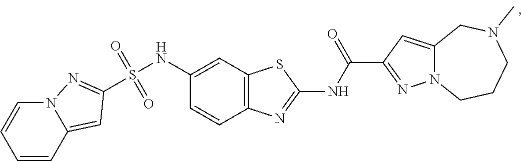 [SMILES: CN1CCCn2nc(C(=O)Nc3nc4ccc(NS(=O)(=O)c5cc6ccccn6n5)cc4s3)cc2C1] | (aj) |
| 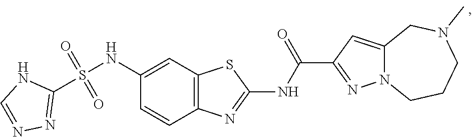 [SMILES: CN1CCCn2nc(C(=O)Nc3nc4ccc(NS(=O)(=O)c5nnc[nH]5)cc4s3)cc2C1] | (ak) |
| 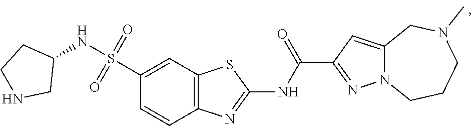 [SMILES: CN1CCCn2nc(C(=O)Nc3nc4ccc(S(=O)(=O)N[C@H]5CCNC5)cc4s3)cc2C1] | (al) |
| 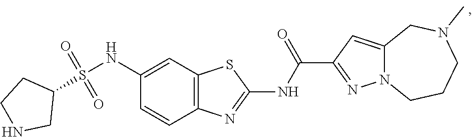 [SMILES: CN1CCCn2nc(C(=O)Nc3nc4ccc(NS(=O)(=O)[C@H]5CCNC5)cc4s3)cc2C1] | (am) |
| 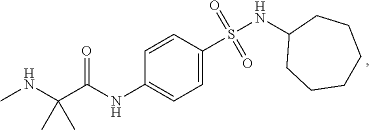 [SMILES: CNC(C)(C)C(=O)Nc1ccc(S(=O)(=O)NC2CCCCCC2)cc1] | (an) |
| 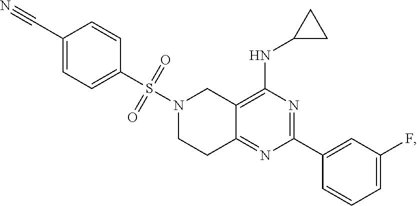 [SMILES: N#Cc1ccc(S(=O)(=O)N2CCc3nc(-c4cccc(F)c4)nc(NC4CC4)c3C2)cc1] | (ao) |
| 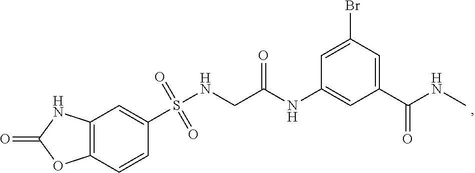 [SMILES: CNC(=O)c1cc(Br)cc(NC(=O)CNS(=O)(=O)c2ccc3oc(=O)[nH]c3c2)c1] | (ap) |
| 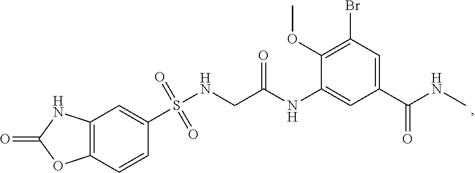 [SMILES: CNC(=O)c1cc(Br)c(OC)c(NC(=O)CNS(=O)(=O)c2ccc3oc(=O)[nH]c3c2)c1] | (ak) |
| 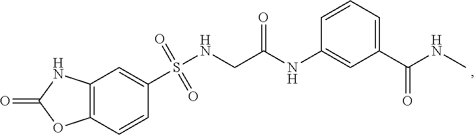 [SMILES: CNC(=O)c1cccc(NC(=O)CNS(=O)(=O)c2ccc3oc(=O)[nH]c3c2)c1] | (ar) |
| 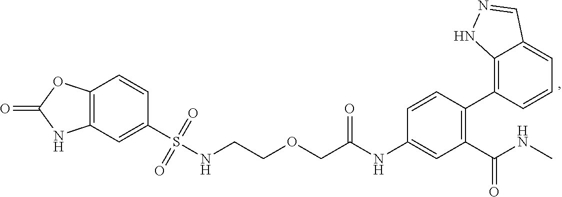 [SMILES: CNC(=O)c1cc(NC(=O)COCCNS(=O)(=O)c2ccc3oc(=O)[nH]c3c2)ccc1-c1cccc2cn[nH]c12] | (as) |
| 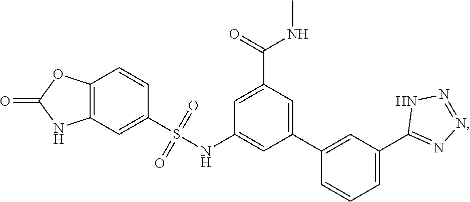 [SMILES: CNC(=O)c1cc(NS(=O)(=O)c2ccc3oc(=O)[nH]c3c2)cc(-c2cccc(-c3nnn[nH]3)c2)c1] | (at) |
| 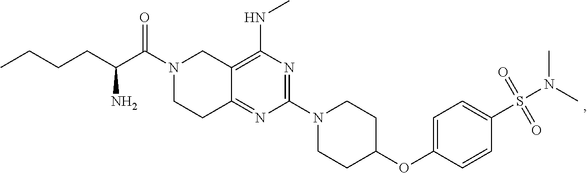 [SMILES: CCCC[C@H](N)C(=O)N1CCc2nc(N3CCC(Oc4ccc(S(=O)(=O)N(C)C)cc4)CC3)nc(NC)c2C1] | (au) |
| 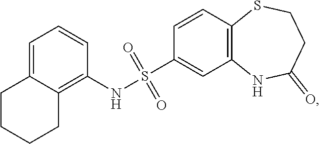 [SMILES: O=C1CCSc2ccc(S(=O)(=O)Nc3cccc4c3CCCC4)cc2N1] | (av) |
| 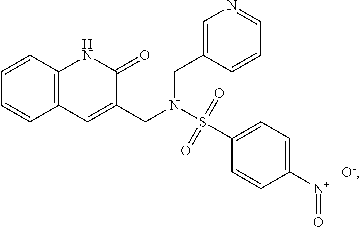 | (aw) |
| 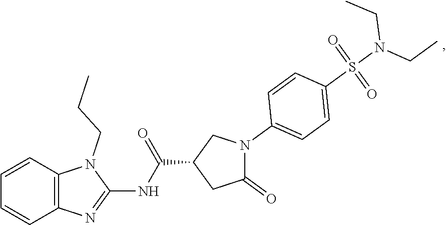 [SMILES: CCCn1c(NC(=O)[C@H]2CC(=O)N(c3ccc(S(=O)(=O)N(CC)CC)cc3)C2)nc2ccccc21] | (ax) |
| 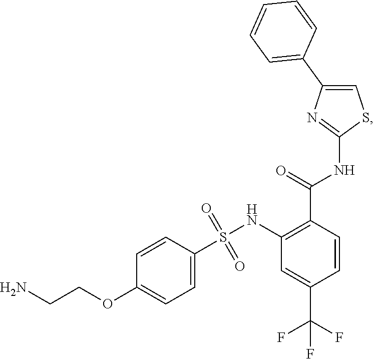 [SMILES: NCCOc1ccc(S(=O)(=O)Nc2cc(C(F)(F)F)ccc2C(=O)Nc2nc(-c3ccccc3)cs2)cc1] | (ay) |
| 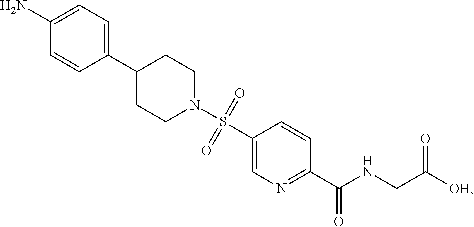 [SMILES: Nc1ccc(C2CCN(S(=O)(=O)c3ccc(C(=O)NCC(=O)O)nc3)CC2)cc1] | (az) |
| 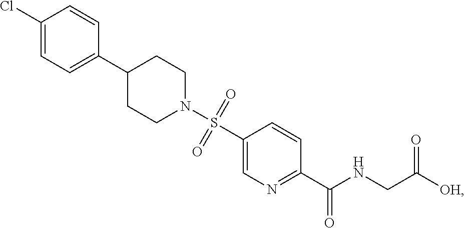 [SMILES: O=C(O)CNC(=O)c1ccc(S(=O)(=O)N2CCC(c3ccc(Cl)cc3)CC2)cn1] | (ba) |
| 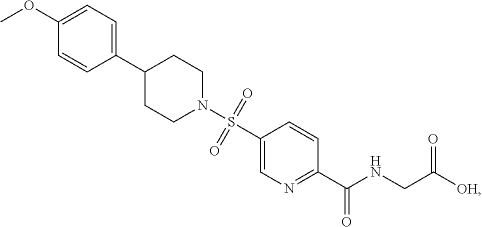 [SMILES: COc1ccc(C2CCN(S(=O)(=O)c3ccc(C(=O)NCC(=O)O)nc3)CC2)cc1] | (bb) |
| 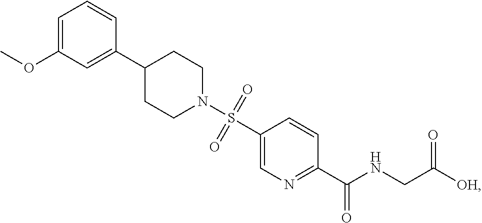 [SMILES: COc1cccc(C2CCN(S(=O)(=O)c3ccc(C(=O)NCC(=O)O)nc3)CC2)c1] | (bc) |
| 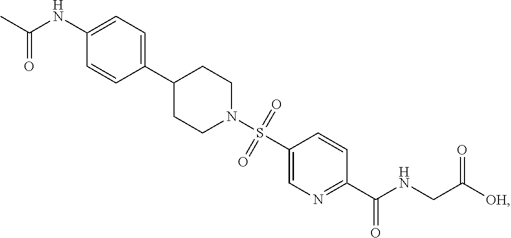 [SMILES: CC(=O)Nc1ccc(C2CCN(S(=O)(=O)c3ccc(C(=O)NCC(=O)O)nc3)CC2)cc1] | (bd) |
| 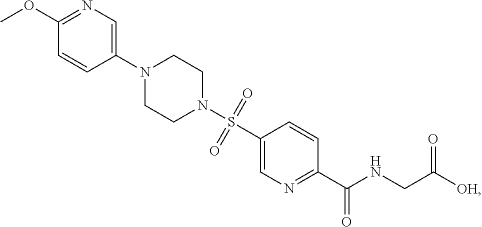 [SMILES: COc1ccc(N2CCN(S(=O)(=O)c3ccc(C(=O)NCC(=O)O)nc3)CC2)cn1] | (be) |
| 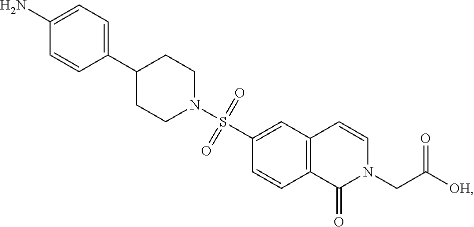 [SMILES: Nc1ccc(C2CCN(S(=O)(=O)c3ccc4c(=O)n(CC(=O)O)ccc4c3)CC2)cc1] | (bf) |
| 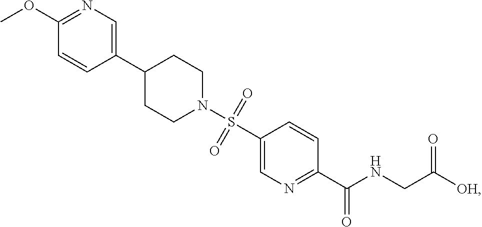 [SMILES: COc1ccc(C2CCN(S(=O)(=O)c3ccc(C(=O)NCC(=O)O)nc3)CC2)cn1] | (bh) |
| 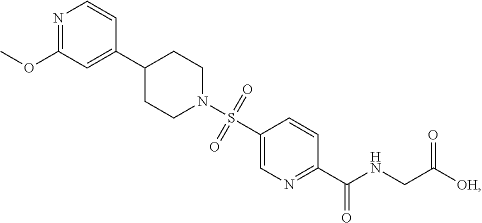 [SMILES: COc1cc(C2CCN(S(=O)(=O)c3ccc(C(=O)NCC(=O)O)nc3)CC2)ccn1] | (bi) |
| 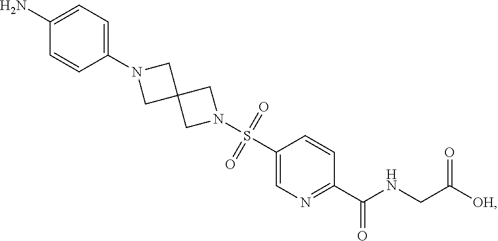 [SMILES: Nc1ccc(N2CC3(C2)CN(S(=O)(=O)c2ccc(C(=O)NCC(=O)O)nc2)C3)cc1] | (bj) |
| 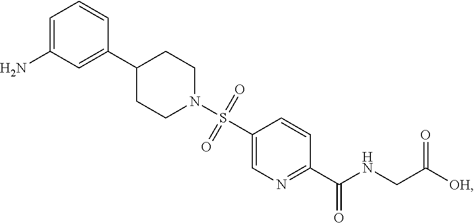 [SMILES: Nc1cccc(C2CCN(S(=O)(=O)c3ccc(C(=O)NCC(=O)O)nc3)CC2)c1] | (bk) |
| 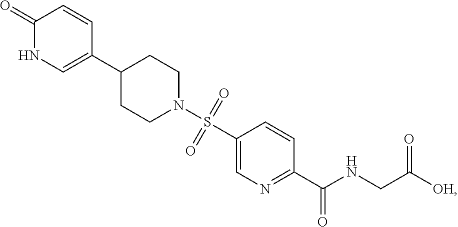 [SMILES: O=C(O)CNC(=O)c1ccc(S(=O)(=O)N2CCC(c3ccc(=O)[nH]c3)CC2)cn1] | (bl) |
| 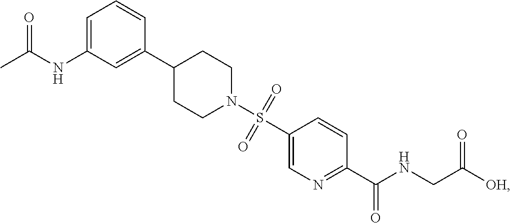 [SMILES: CC(=O)Nc1cccc(C2CCN(S(=O)(=O)c3ccc(C(=O)NCC(=O)O)nc3)CC2)c1] | (bm) |
| 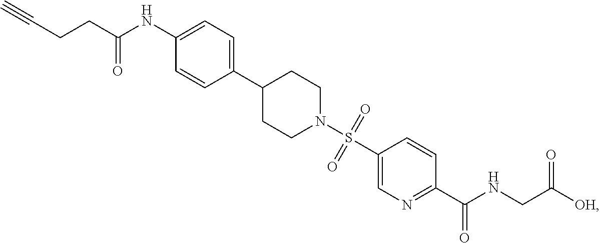 [SMILES: C#CCCC(=O)Nc1ccc(C2CCN(S(=O)(=O)c3ccc(C(=O)NCC(=O)O)nc3)CC2)cc1] | (bn) |
| 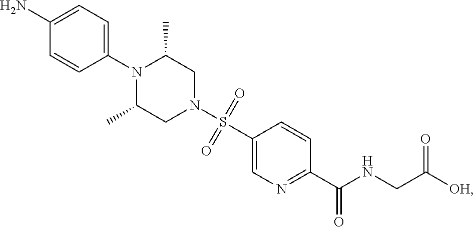 [SMILES: C[C@@H]1CN(S(=O)(=O)c2ccc(C(=O)NCC(=O)O)nc2)C[C@H](C)N1c1ccc(N)cc1] | (bo) |
| 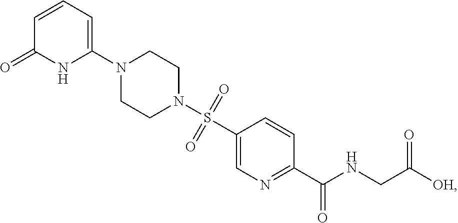 [SMILES: O=C(O)CNC(=O)c1ccc(S(=O)(=O)N2CCN(c3cccc(=O)[nH]3)CC2)cn1] | (bp) |
| 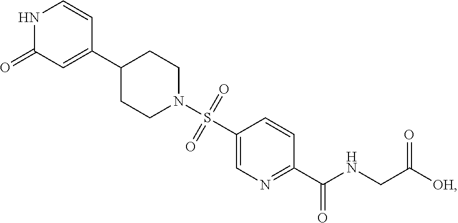 [SMILES: O=C(O)CNC(=O)c1ccc(S(=O)(=O)N2CCC(c3cc[nH]c(=O)c3)CC2)cn1] | (bq) |
| 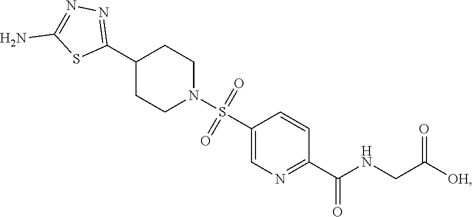 [SMILES: Nc1nnc(C2CCN(S(=O)(=O)c3ccc(C(=O)NCC(=O)O)nc3)CC2)s1] | (br) |
| 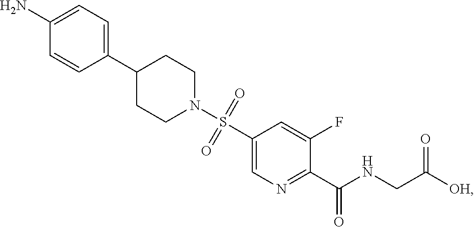 [SMILES: Nc1ccc(C2CCN(S(=O)(=O)c3cnc(C(=O)NCC(=O)O)c(F)c3)CC2)cc1] | (bs) |
| 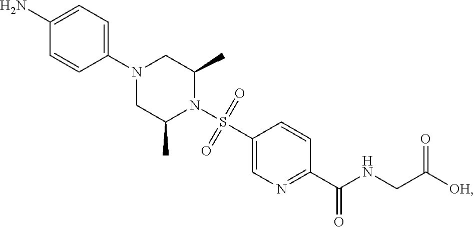 [SMILES: C[C@@H]1CN(c2ccc(N)cc2)C[C@H](C)N1S(=O)(=O)c1ccc(C(=O)NCC(=O)O)nc1] | (bt) |
| 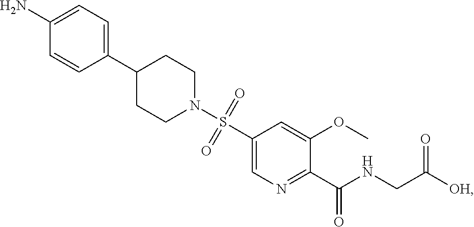 [SMILES: COc1cc(S(=O)(=O)N2CCC(c3ccc(N)cc3)CC2)cnc1C(=O)NCC(=O)O] | (bu) |
| 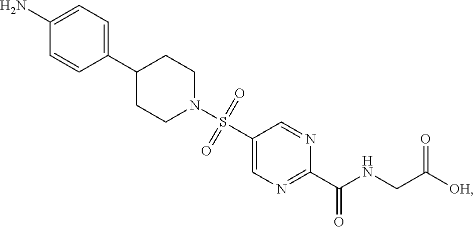 [SMILES: Nc1ccc(C2CCN(S(=O)(=O)c3cnc(C(=O)NCC(=O)O)nc3)CC2)cc1] | (bv) |
| 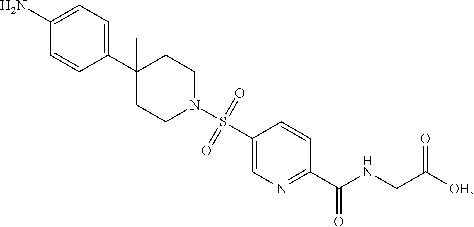 [SMILES: CC1(c2ccc(N)cc2)CCN(S(=O)(=O)c2ccc(C(=O)NCC(=O)O)nc2)CC1] | (bw) |
| 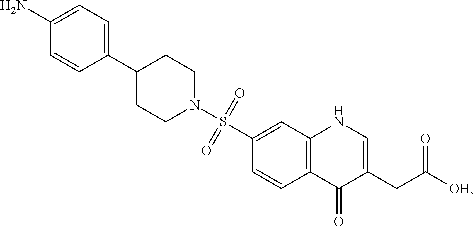 [SMILES: Nc1ccc(C2CCN(S(=O)(=O)c3ccc4c(=O)c(CC(=O)O)c[nH]c4c3)CC2)cc1] | (bx) |
| 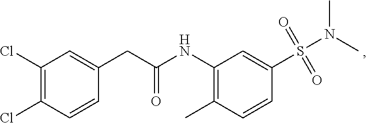 [SMILES: Cc1ccc(S(=O)(=O)N(C)C)cc1NC(=O)Cc1ccc(Cl)c(Cl)c1] | (by) |
| 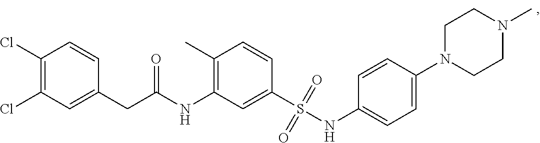 [SMILES: Cc1ccc(S(=O)(=O)Nc2ccc(N3CCN(C)CC3)cc2)cc1NC(=O)Cc1ccc(Cl)c(Cl)c1] | (bz) |
| 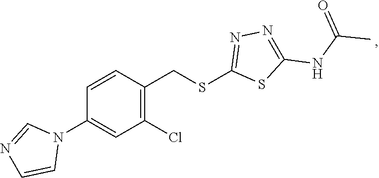 [SMILES: CC(=O)Nc1nnc(SCc2ccc(-n3ccnc3)cc2Cl)s1] | (ca) |
| 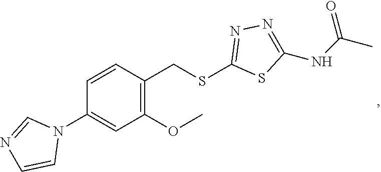 [SMILES: COc1cc(-n2ccnc2)ccc1CSc1nnc(NC(C)=O)s1] | (cb) |
| 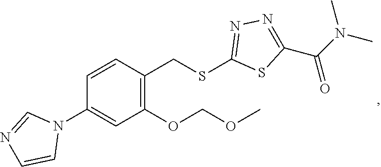 [SMILES: COCOc1cc(-n2ccnc2)ccc1CSc1nnc(C(=O)N(C)C)s1] | (cc) |
| 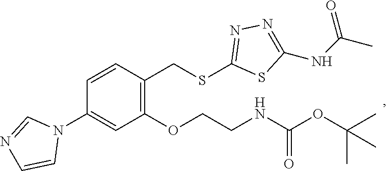 [SMILES: CC(=O)Nc1nnc(SCc2ccc(-n3ccnc3)cc2OCCNC(=O)OC(C)(C)C)s1] | (cd) |
| 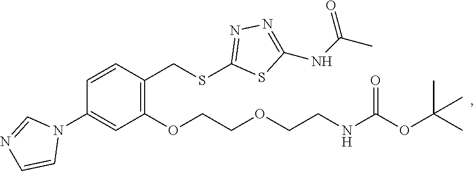 [SMILES: CC(=O)Nc1nnc(SCc2ccc(-n3ccnc3)cc2OCCOCCNC(=O)OC(C)(C)C)s1] | (ce) |
| 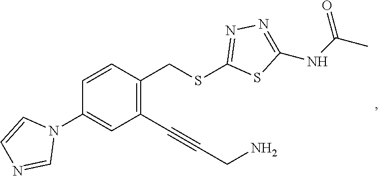 [SMILES: CC(=O)Nc1nnc(SCc2ccc(-n3ccnc3)cc2C#CCN)s1] | (cf) |
| 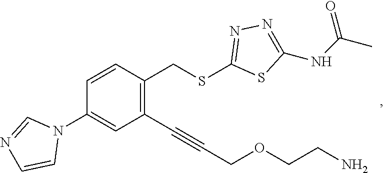 [SMILES: CC(=O)Nc1nnc(SCc2ccc(-n3ccnc3)cc2C#CCOCCN)s1] | (cg) |
| 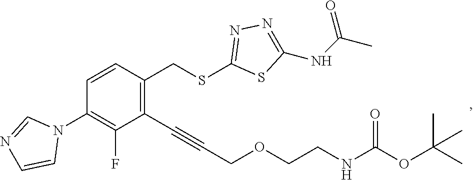 [SMILES: CC(=O)Nc1nnc(SCc2ccc(-n3ccnc3)c(F)c2C#CCOCCNC(=O)OC(C)(C)C)s1] | (ch) |
| 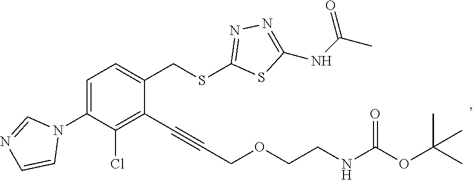 [SMILES: CC(=O)Nc1nnc(SCc2ccc(-n3ccnc3)c(Cl)c2C#CCOCCNC(=O)OC(C)(C)C)s1] | (ci) |
| 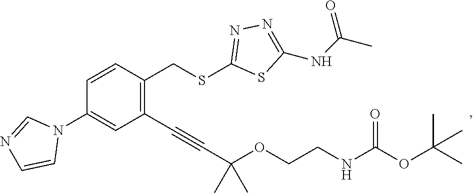 [SMILES: CC(=O)Nc1nnc(SCc2ccc(-n3ccnc3)cc2C#CC(C)(C)OCCNC(=O)OC(C)(C)C)s1] | (cj) |
| 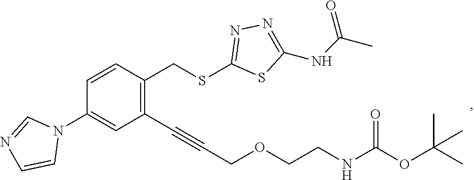 [SMILES: CC(=O)Nc1nnc(SCc2ccc(-n3ccnc3)cc2C#CCOCCNC(=O)OC(C)(C)C)s1] | (ck) |
| 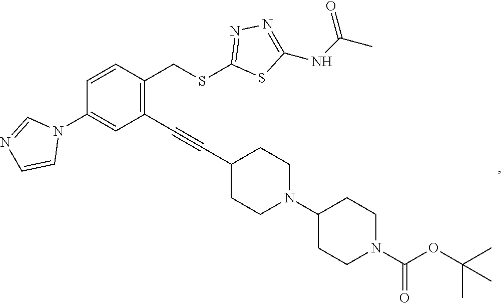 [SMILES: CC(=O)Nc1nnc(SCc2ccc(-n3ccnc3)cc2C#CC2CCN(C3CCN(C(=O)OC(C)(C)C)CC3)CC2)s1] | (cl) |
| 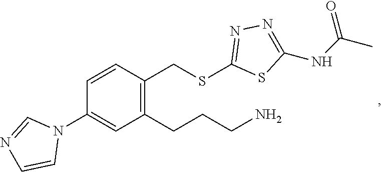 [SMILES: CC(=O)Nc1nnc(SCc2ccc(-n3ccnc3)cc2CCCN)s1] | (cm) |
| 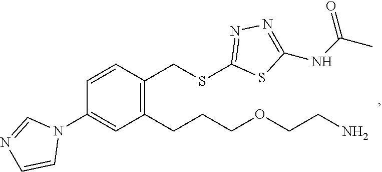 [SMILES: CC(=O)Nc1nnc(SCc2ccc(-n3ccnc3)cc2CCCOCCN)s1] | (cn) |
| 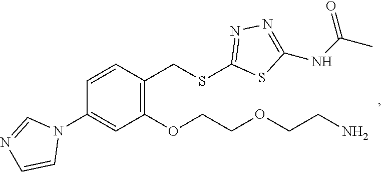 [SMILES: CC(=O)Nc1nnc(SCc2ccc(-n3ccnc3)cc2OCCOCCN)s1] | (co) |
| 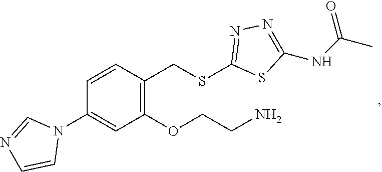 [SMILES: CC(=O)Nc1nnc(SCc2ccc(-n3ccnc3)cc2OCCN)s1] | (cp) |
| 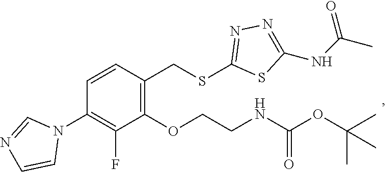 [SMILES: CC(=O)Nc1nnc(SCc2ccc(-n3ccnc3)c(F)c2OCCNC(=O)OC(C)(C)C)s1] | (cq) |
| 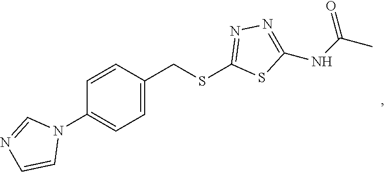 [SMILES: CC(=O)Nc1nnc(SCc2ccc(-n3ccnc3)cc2)s1] | (cr) |
| 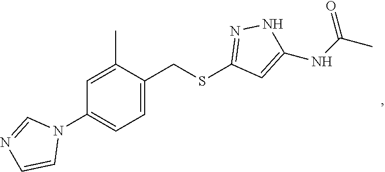 [SMILES: CC(=O)Nc1cc(SCc2ccc(-n3ccnc3)cc2C)n[nH]1] | (cs) |
| 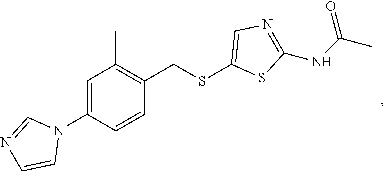 [SMILES: CC(=O)Nc1ncc(SCc2ccc(-n3ccnc3)cc2C)s1] | (ct) |
| 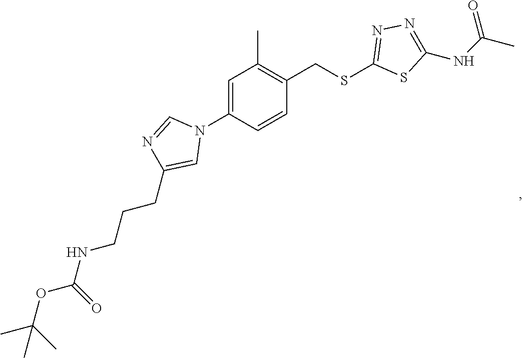 [SMILES: CC(=O)Nc1nnc(SCc2ccc(-n3cnc(CCCNC(=O)OC(C)(C)C)c3)cc2C)s1] | (cu) |
| 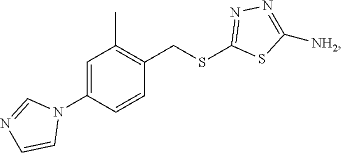 [SMILES: Cc1cc(-n2ccnc2)ccc1CSc1nnc(N)s1] | (cv) |
| 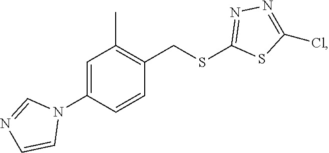 [SMILES: Cc1cc(-n2ccnc2)ccc1CSc1nnc(Cl)s1] | (cw) |
| 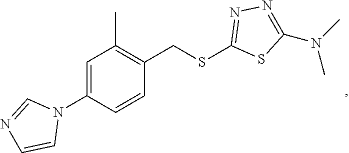 [SMILES: Cc1cc(-n2ccnc2)ccc1CSc1nnc(N(C)C)s1] | (cx) |
| 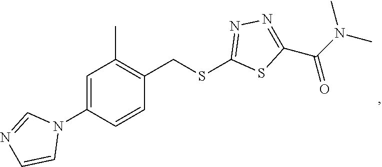 [SMILES: Cc1cc(-n2ccnc2)ccc1CSc1nnc(C(=O)N(C)C)s1] | (cy) |
| 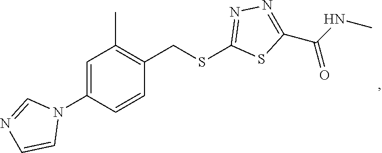 [SMILES: CNC(=O)c1nnc(SCc2ccc(-n3ccnc3)cc2C)s1] | (cz) |
| 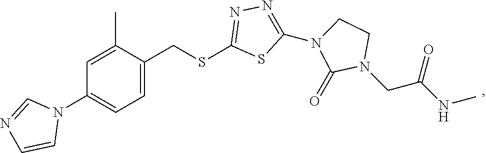 [SMILES: CNC(=O)CN1CCN(c2nnc(SCc3ccc(-n4ccnc4)cc3C)s2)C1=O] | (da) |
| 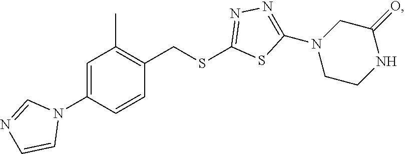 [SMILES: Cc1cc(-n2ccnc2)ccc1CSc1nnc(N2CCNC(=O)C2)s1] | (db) |
| 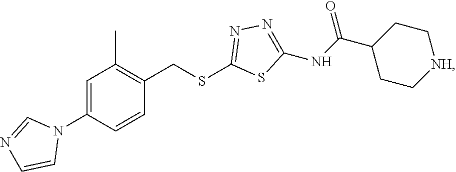 [SMILES: Cc1cc(-n2ccnc2)ccc1CSc1nnc(NC(=O)C2CCNCC2)s1] | (dc) |
| 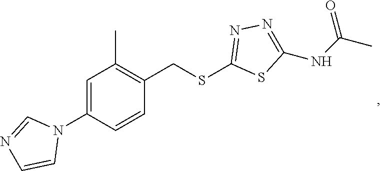 [SMILES: CC(=O)Nc1nnc(SCc2ccc(-n3ccnc3)cc2C)s1] | (dd) |
| 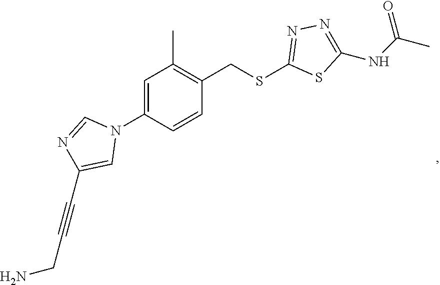 [SMILES: CC(=O)Nc1nnc(SCc2ccc(-n3cnc(C#CCN)c3)cc2C)s1] | (de) |
| 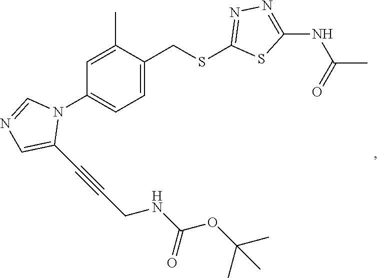 [SMILES: CC(=O)Nc1nnc(SCc2ccc(-n3cncc3C#CCNC(=O)OC(C)(C)C)cc2C)s1] | (df) |
| 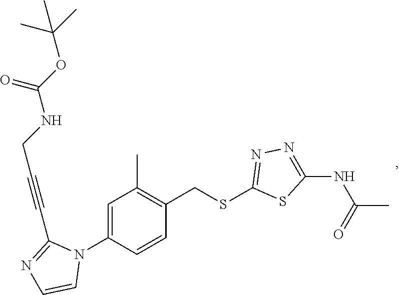 [SMILES: CC(=O)Nc1nnc(SCc2ccc(-n3ccnc3C#CCNC(=O)OC(C)(C)C)cc2C)s1] | (dg) |
| 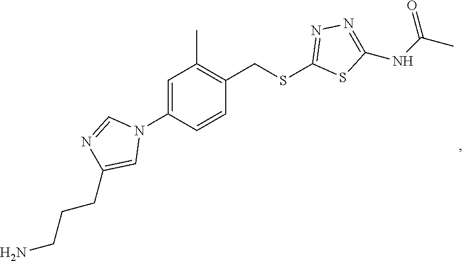 [SMILES: CC(=O)Nc1nnc(SCc2ccc(-n3cnc(CCCN)c3)cc2C)s1] | (dh) |
| 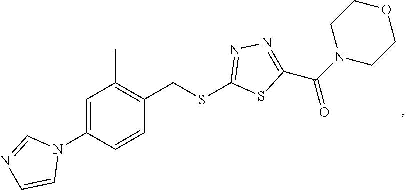 [SMILES: Cc1cc(-n2ccnc2)ccc1CSc1nnc(C(=O)N2CCOCC2)s1] | (di) |
| 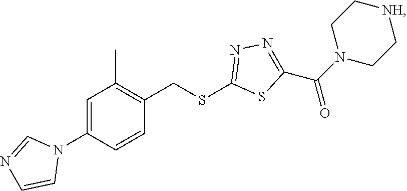 [SMILES: Cc1cc(-n2ccnc2)ccc1CSc1nnc(C(=O)N2CCNCC2)s1] | (dj) |
| 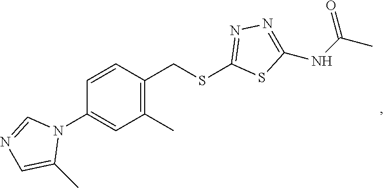 [SMILES: CC(=O)Nc1nnc(SCc2ccc(-n3cncc3C)cc2C)s1] | (dk) |
| 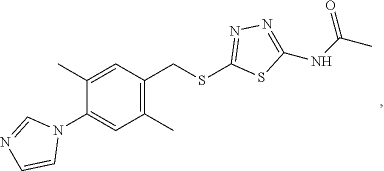 [SMILES: CC(=O)Nc1nnc(SCc2cc(C)c(-n3ccnc3)cc2C)s1] | (dl) |
| 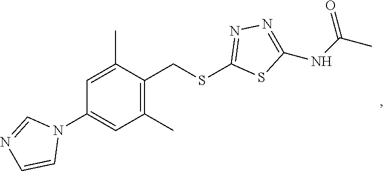 [SMILES: CC(=O)Nc1nnc(SCc2c(C)cc(-n3ccnc3)cc2C)s1] | (dm) |
| 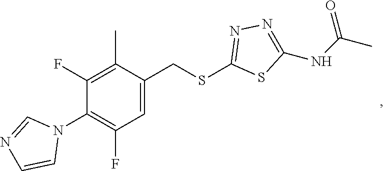 [SMILES: CC(=O)Nc1nnc(SCc2cc(F)c(-n3ccnc3)c(F)c2C)s1] | (dn) |
| 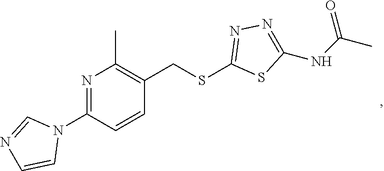 [SMILES: CC(=O)Nc1nnc(SCc2ccc(-n3ccnc3)nc2C)s1] | (do) |
| 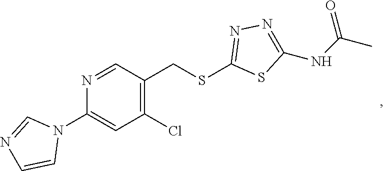 [SMILES: CC(=O)Nc1nnc(SCc2cnc(-n3ccnc3)cc2Cl)s1] | (dp) |
| 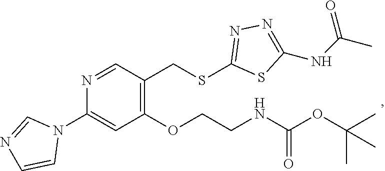 [SMILES: CC(=O)Nc1nnc(SCc2cnc(-n3ccnc3)cc2OCCNC(=O)OC(C)(C)C)s1] | (dq) |
| 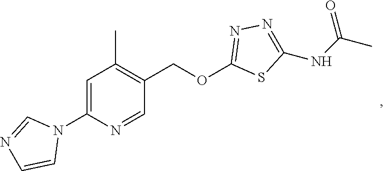 [SMILES: CC(=O)Nc1nnc(OCc2cnc(-n3ccnc3)cc2C)s1] | (dr) |
| 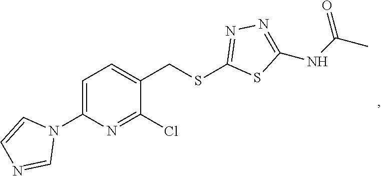 [SMILES: CC(=O)Nc1nnc(SCc2ccc(-n3ccnc3)nc2Cl)s1] | (ds) |
| 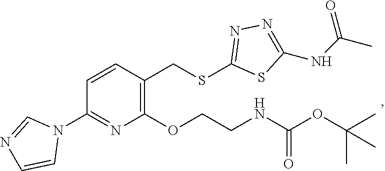 [SMILES: CC(=O)Nc1nnc(SCc2ccc(-n3ccnc3)nc2OCCNC(=O)OC(C)(C)C)s1] | (dt) |
| 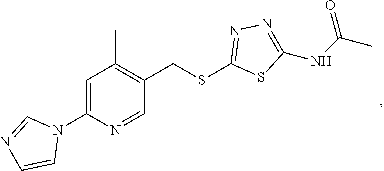 [SMILES: CC(=O)Nc1nnc(SCc2cnc(-n3ccnc3)cc2C)s1] | (du) |
| 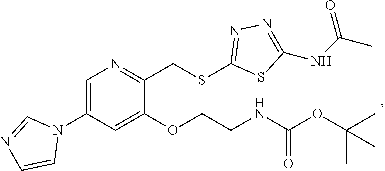 [SMILES: CC(=O)Nc1nnc(SCc2ncc(-n3ccnc3)cc2OCCNC(=O)OC(C)(C)C)s1] | (dv) |
| 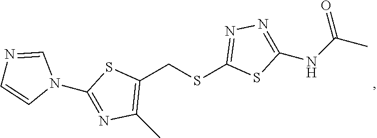 [SMILES: CC(=O)Nc1nnc(SCc2sc(-n3ccnc3)nc2C)s1] | (dw) |
| 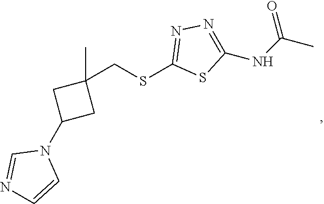 [SMILES: CC(=O)Nc1nnc(SCC2(C)CC(n3ccnc3)C2)s1] | (dx) |
| 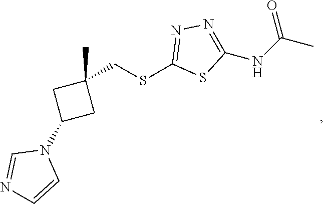 [SMILES: CC(=O)Nc1nnc(SC[C@]2(C)C[C@H](n3ccnc3)C2)s1] | (dy) |
| 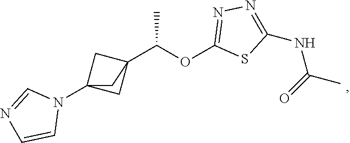 [SMILES: CC(=O)Nc1nnc(O[C@@H](C)C23CC(n4ccnc4)(C2)C3)s1] | (dz) |
| 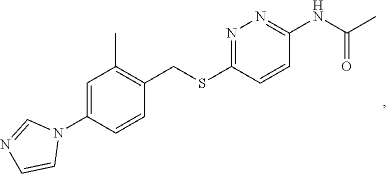 [SMILES: CC(=O)Nc1ccc(SCc2ccc(-n3ccnc3)cc2C)nn1] | (ea) |
| 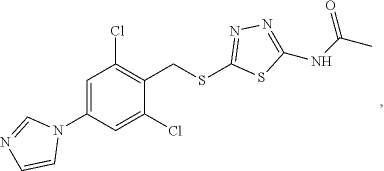 [SMILES: CC(=O)Nc1nnc(SCc2c(Cl)cc(-n3ccnc3)cc2Cl)s1] | (eb) |
| 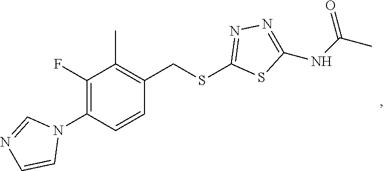 [SMILES: CC(=O)Nc1nnc(SCc2ccc(-n3ccnc3)c(F)c2C)s1] | (ec) |
| 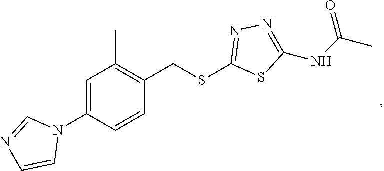 [SMILES: CC(=O)Nc1nnc(SCc2ccc(-n3ccnc3)cc2C)s1] | (ed) |
| 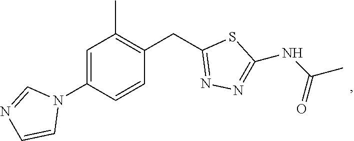 [SMILES: CC(=O)Nc1nnc(Cc2ccc(-n3ccnc3)cc2C)s1] | (ee) |
| 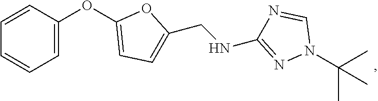 [SMILES: CC(C)(C)n1cnc(NCc2ccc(Oc3ccccc3)o2)n1] | (ef) |
| 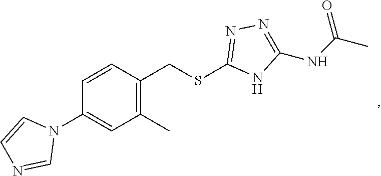 [SMILES: CC(=O)Nc1nnc(SCc2ccc(-n3ccnc3)cc2C)[nH]1] | (eg) |
| 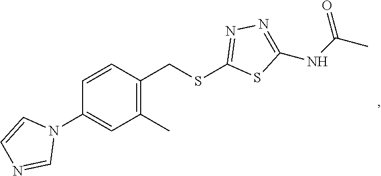 [SMILES: CC(=O)Nc1nnc(SCc2ccc(-n3ccnc3)cc2C)s1] | (eh) |
| 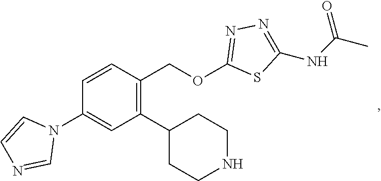 [SMILES: CC(=O)Nc1nnc(OCc2ccc(-n3ccnc3)cc2C2CCNCC2)s1] | (ei) |
| 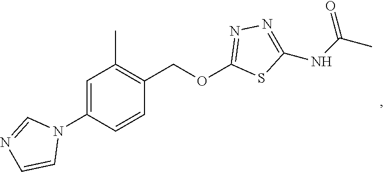 [SMILES: CC(=O)Nc1nnc(OCc2ccc(-n3ccnc3)cc2C)s1] | (ej) |
| 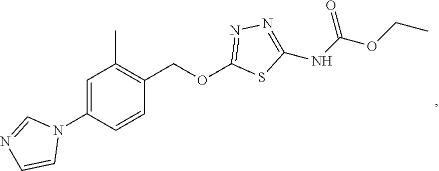 [SMILES: CCOC(=O)Nc1nnc(OCc2ccc(-n3ccnc3)cc2C)s1] | (ek) |
| 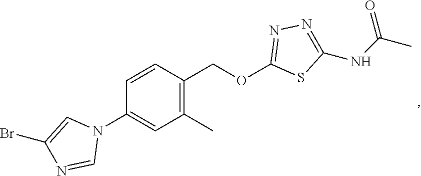 [SMILES: CC(=O)Nc1nnc(OCc2ccc(-n3cnc(Br)c3)cc2C)s1] | (el) |
| 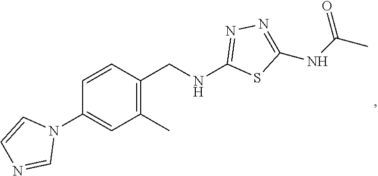 [SMILES: CC(=O)Nc1nnc(NCc2ccc(-n3ccnc3)cc2C)s1] | (em) |
| 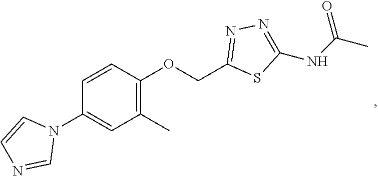 [SMILES: CC(=O)Nc1nnc(COc2ccc(-n3ccnc3)cc2C)s1] | (en) |
| 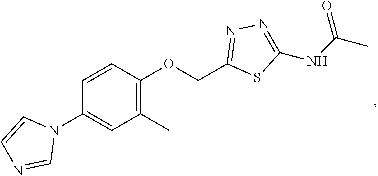 [SMILES: CC(=O)Nc1nnc(COc2ccc(-n3ccnc3)cc2C)s1] | (eo) |
| 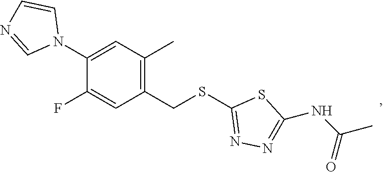 [SMILES: CC(=O)Nc1nnc(SCc2cc(F)c(-n3ccnc3)cc2C)s1] | (ep) |
| 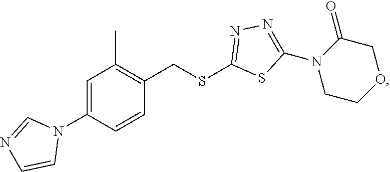 [SMILES: Cc1cc(-n2ccnc2)ccc1CSc1nnc(N2CCOCC2=O)s1] | (eq) |
| 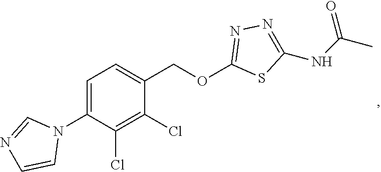 [SMILES: CC(=O)Nc1nnc(OCc2ccc(-n3ccnc3)c(Cl)c2Cl)s1] | (er) |
| 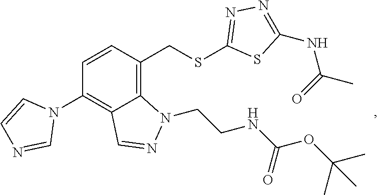 [SMILES: CC(=O)Nc1nnc(SCc2ccc(-n3ccnc3)c3cnn(CCNC(=O)OC(C)(C)C)c23)s1] | (es) |
| 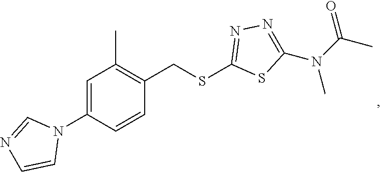 [SMILES: CC(=O)N(C)c1nnc(SCc2ccc(-n3ccnc3)cc2C)s1] | (et) |
| 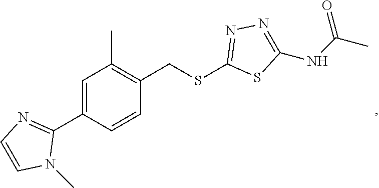 [SMILES: CC(=O)Nc1nnc(SCc2ccc(-c3nccn3C)cc2C)s1] | (eu) |
| 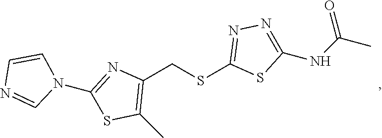 [SMILES: CC(=O)Nc1nnc(SCc2nc(-n3ccnc3)sc2C)s1] | (ev) |
| 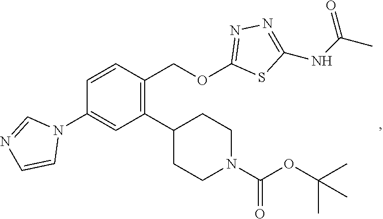 [SMILES: CC(=O)Nc1nnc(OCc2ccc(-n3ccnc3)cc2C2CCN(C(=O)OC(C)(C)C)CC2)s1] | (ew) |
| 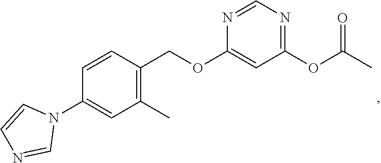 [SMILES: CC(=O)Oc1cc(OCc2ccc(-n3ccnc3)cc2C)ncn1] | (ex) |
| 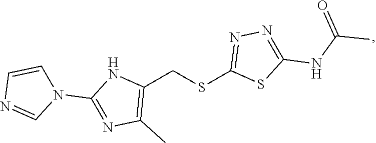 [SMILES: CC(=O)Nc1nnc(SCc2[nH]c(-n3ccnc3)nc2C)s1] | (ey) |
| 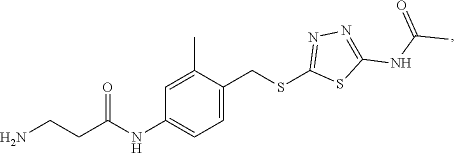 [SMILES: CC(=O)Nc1nnc(SCc2ccc(NC(=O)CCN)cc2C)s1] | (ez) |
| 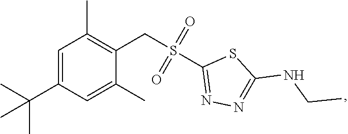 [SMILES: CCNc1nnc(S(=O)(=O)Cc2c(C)cc(C(C)(C)C)cc2C)s1] | (fa) |
| 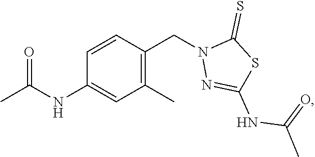 [SMILES: CC(=O)Nc1ccc(Cn2nc(NC(C)=O)sc2=S)c(C)c1] | (fb) |
| 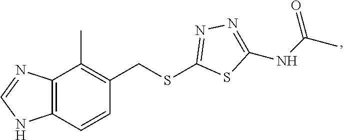 [SMILES: CC(=O)Nc1nnc(SCc2ccc3[nH]cnc3c2C)s1] | (fc) |
| 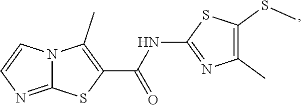 [SMILES: CSc1sc(NC(=O)c2sc3nccn3c2C)nc1C] | (fd) |
| 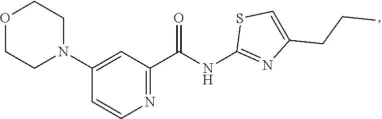 [SMILES: CCCc1csc(NC(=O)c2cc(N3CCOCC3)ccn2)n1] | (fe) |
| 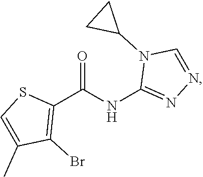 [SMILES: Cc1csc(C(=O)Nc2nncn2C2CC2)c1Br] | (ff) |
| 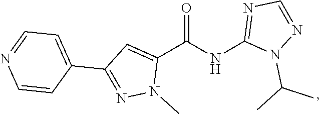 [SMILES: CC(C)n1ncnc1NC(=O)c1cc(-c2ccncc2)nn1C] | (fg) |
| 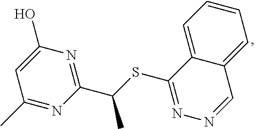 [SMILES: Cc1cc(O)nc([C@H](C)Sc2nncc3ccccc23)n1] | (fh) |
| 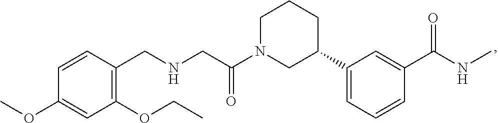 | (fi) |
| 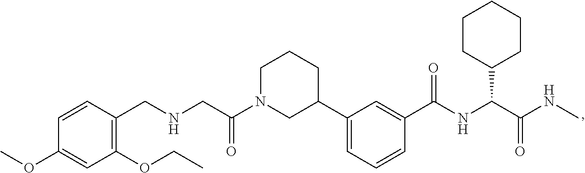 | (fj) |
| 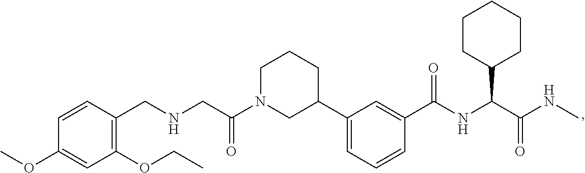 | (fk) |
| 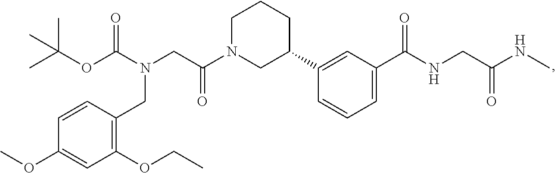 | (fl) |
| 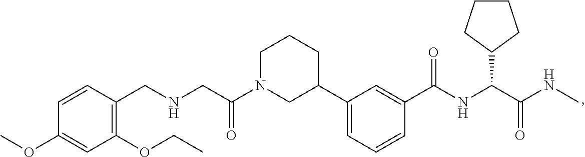 | (fm) |
| 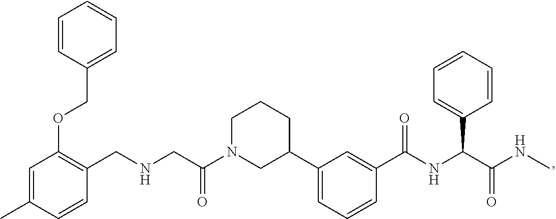 | (fn) |
| 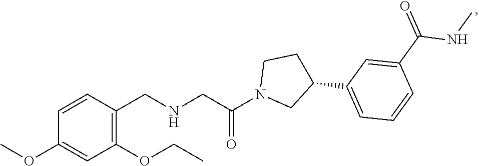 | (fo) |
| 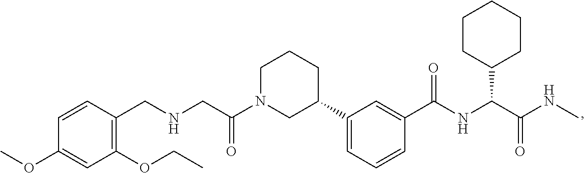 | (fp) |
| 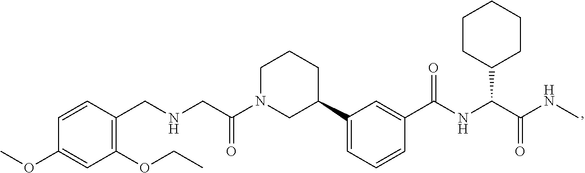 | (fq) |
| 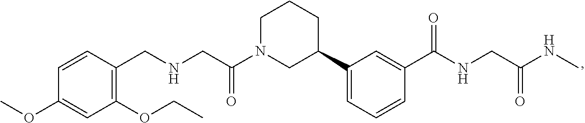 | (fr) |
| 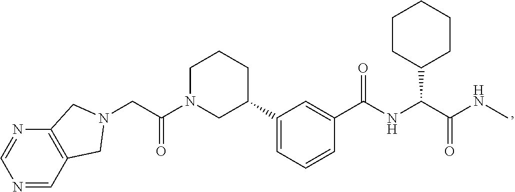 [SMILES: CNC(=O)[C@H](NC(=O)c1cccc([C@H]2CCCN(C(=O)CN3Cc4cncnc4C3)C2)c1)C1CCCCC1] | (fs) |
| 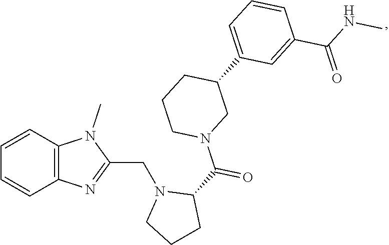 [SMILES: CNC(=O)c1cccc([C@H]2CCCN(C(=O)[C@@H]3CCCN3Cc3nc4ccccc4n3C)C2)c1] | (ft) |
| 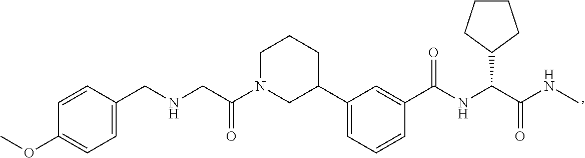 | (fu) |
| 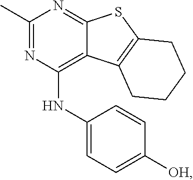 [SMILES: Cc1nc(Nc2ccc(O)cc2)c2c3c(sc2n1)CCCC3] | (fv) |
| 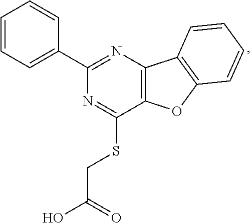 [SMILES: O=C(O)CSc1nc(-c2ccccc2)nc2c1oc1ccccc12] | (fw) |
| 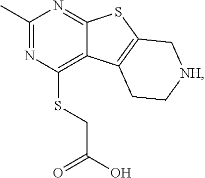 [SMILES: Cc1nc(SCC(=O)O)c2c3c(sc2n1)CNCC3] | (fx) |
| 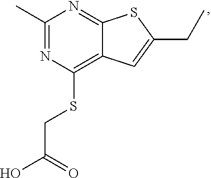 [SMILES: CCc1cc2c(SCC(=O)O)nc(C)nc2s1] | (fy) |
| 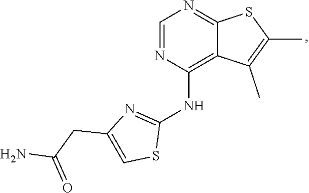 [SMILES: Cc1sc2ncnc(Nc3nc(CC(N)=O)cs3)c2c1C] | (fz) |
| 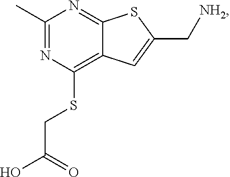 [SMILES: Cc1nc(SCC(=O)O)c2cc(CN)sc2n1] | (ga) |
| 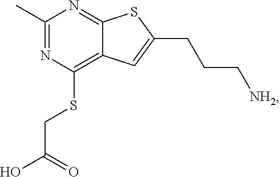 [SMILES: Cc1nc(SCC(=O)O)c2cc(CCCN)sc2n1] | (gb) |
| 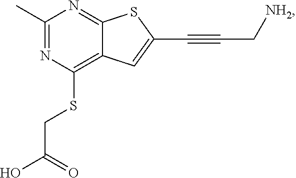 [SMILES: Cc1nc(SCC(=O)O)c2cc(C#CCN)sc2n1] | (gc) |
| 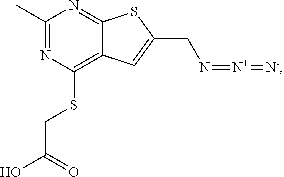 | (gd) |
| 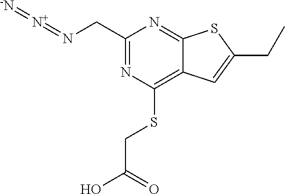 | (ge) |
| 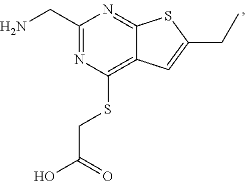 [SMILES: CCc1cc2c(SCC(=O)O)nc(CN)nc2s1] | (gf) |
| 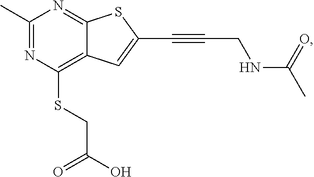 [SMILES: CC(=O)NCC#Cc1cc2c(SCC(=O)O)nc(C)nc2s1] | (gg) |
| 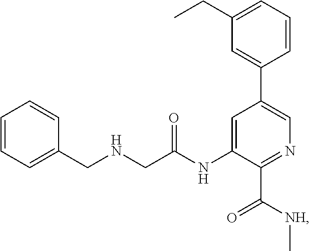 [SMILES: CCc1cccc(-c2cnc(C(=O)NC)c(NC(=O)CNCc3ccccc3)c2)c1] | (hf) |
| 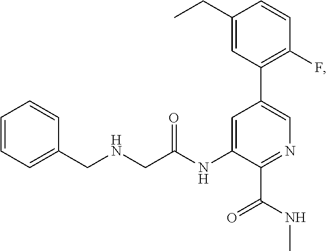 [SMILES: CCc1ccc(F)c(-c2cnc(C(=O)NC)c(NC(=O)CNCc3ccccc3)c2)c1] | (hg) |
| 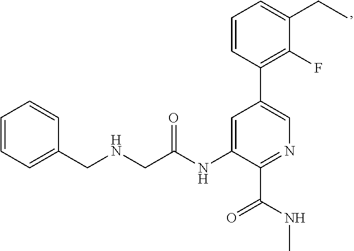 [SMILES: CCc1cccc(-c2cnc(C(=O)NC)c(NC(=O)CNCc3ccccc3)c2)c1F] | (hh) |
| 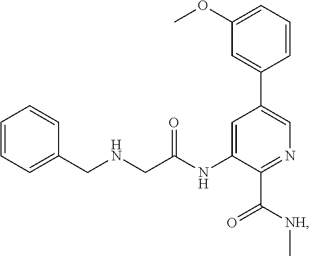 [SMILES: CNC(=O)c1ncc(-c2cccc(OC)c2)cc1NC(=O)CNCc1ccccc1] | (hi) |
| 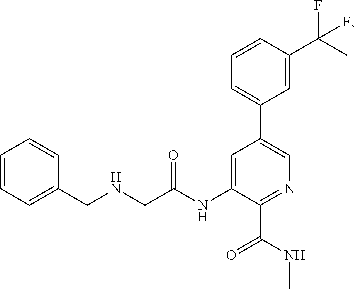 [SMILES: CNC(=O)c1ncc(-c2cccc(C(C)(F)F)c2)cc1NC(=O)CNCc1ccccc1] | (hj) |
| 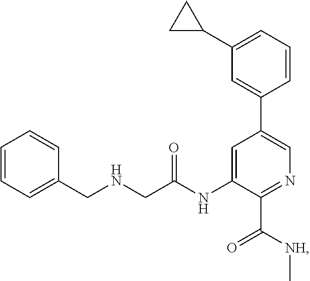 [SMILES: CNC(=O)c1ncc(-c2cccc(C3CC3)c2)cc1NC(=O)CNCc1ccccc1] | (hk) |
| 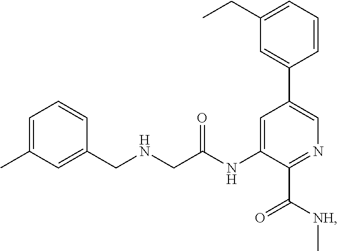 | (hl) |
| 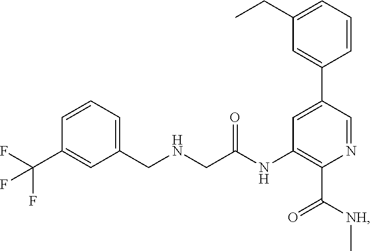 [SMILES: CCc1cccc(-c2cnc(C(=O)NC)c(NC(=O)CNCc3cccc(C(F)(F)F)c3)c2)c1] | (hm) |
| 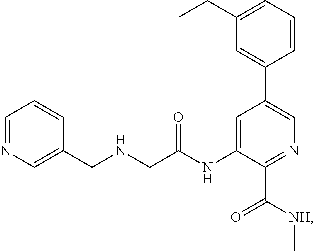 [SMILES: CCc1cccc(-c2cnc(C(=O)NC)c(NC(=O)CNCc3cccnc3)c2)c1] | (hn) |
| 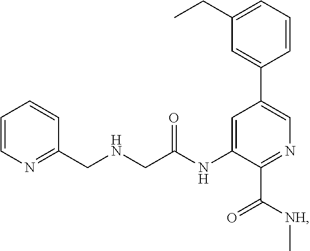 [SMILES: CCc1cccc(-c2cnc(C(=O)NC)c(NC(=O)CNCc3ccccn3)c2)c1] | (ho) |
| 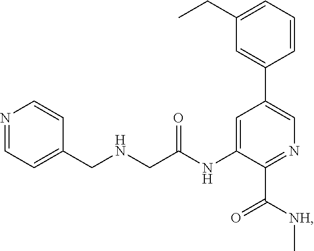 [SMILES: CCc1cccc(-c2cnc(C(=O)NC)c(NC(=O)CNCc3ccncc3)c2)c1] | (hp) |
| 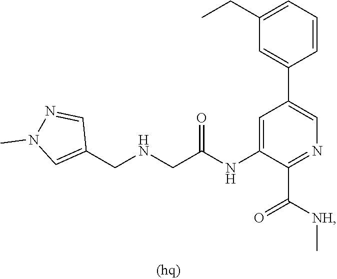 | (hq) |
| 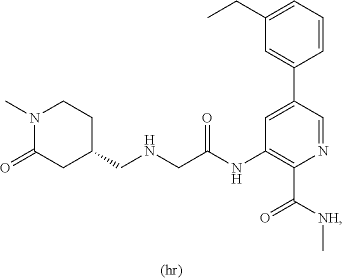 [SMILES: CCc1cccc(-c2cnc(C(=O)NC)c(NC(=O)CNC[C@H]3CCN(C)C(=O)C3)c2)c1] | (hr) |
| 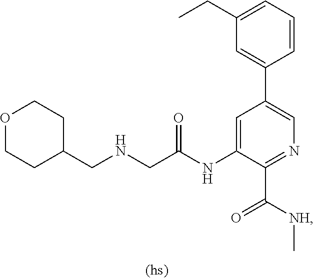 [SMILES: CCc1cccc(-c2cnc(C(=O)NC)c(NC(=O)CNCC3CCOCC3)c2)c1] | (hs) |
| 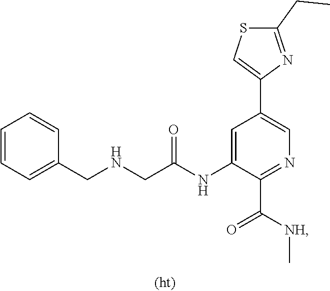 [SMILES: CCc1nc(-c2cnc(C(=O)NC)c(NC(=O)CNCc3ccccc3)c2)cs1] | (ht) |
| 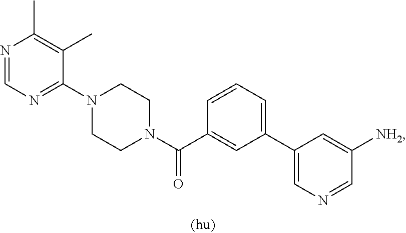 [SMILES: Cc1ncnc(N2CCN(C(=O)c3cccc(-c4cncc(N)c4)c3)CC2)c1C] | (hu) |
| 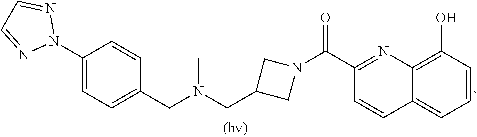 [SMILES: CN(Cc1ccc(-n2nccn2)cc1)CC1CN(C(=O)c2ccc3cccc(O)c3n2)C1] | (hv) |
| 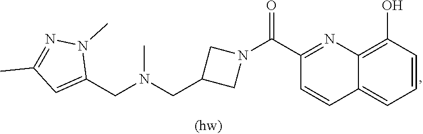 [SMILES: Cc1cc(CN(C)CC2CN(C(=O)c3ccc4cccc(O)c4n3)C2)n(C)n1] | (hw) |
| 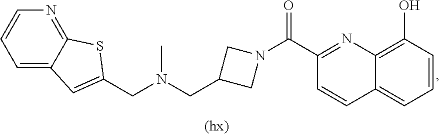 [SMILES: CN(Cc1cc2cccnc2s1)CC1CN(C(=O)c2ccc3cccc(O)c3n2)C1] | (hx) |
| 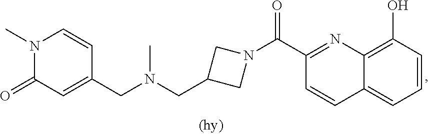 [SMILES: CN(Cc1ccn(C)c(=O)c1)CC1CN(C(=O)c2ccc3cccc(O)c3n2)C1] | (hy) |
| 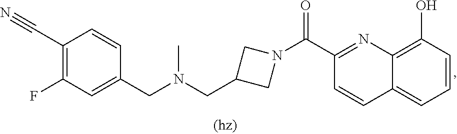 [SMILES: CN(Cc1ccc(C#N)c(F)c1)CC1CN(C(=O)c2ccc3cccc(O)c3n2)C1] | (hz) |
| 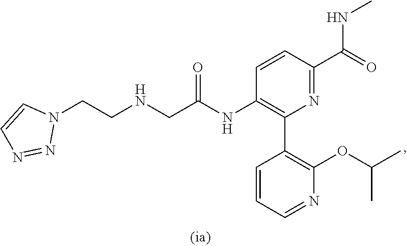 [SMILES: CNC(=O)c1ccc(NC(=O)CNCCn2ccnn2)c(-c2cccnc2OC(C)C)n1] | (ia) |
| 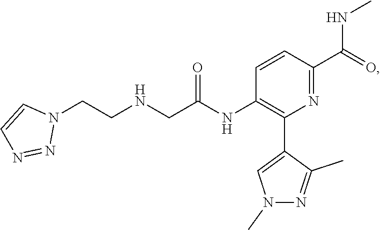 [SMILES: CNC(=O)c1ccc(NC(=O)CNCCn2ccnn2)c(-c2cn(C)nc2C)n1] | (ib) |
| 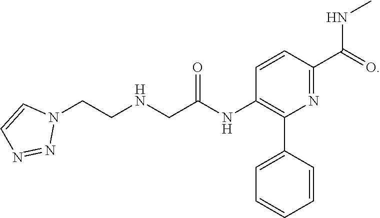 [SMILES: CNC(=O)c1ccc(NC(=O)CNCCn2ccnn2)c(-c2ccccc2)n1] | (ic) |
| 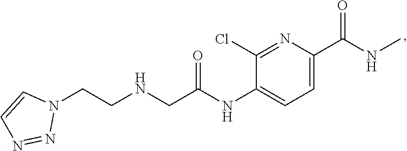 [SMILES: CNC(=O)c1ccc(NC(=O)CNCCn2ccnn2)c(Cl)n1] | (id) |
| 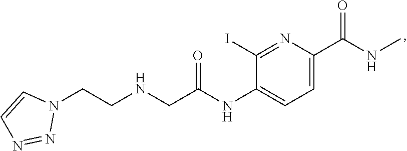 [SMILES: CNC(=O)c1ccc(NC(=O)CNCCn2ccnn2)c(I)n1] | (ie) |
| 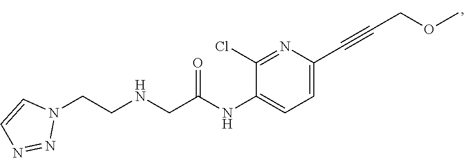 [SMILES: COCC#Cc1ccc(NC(=O)CNCCn2ccnn2)c(Cl)n1] | (if) |
| 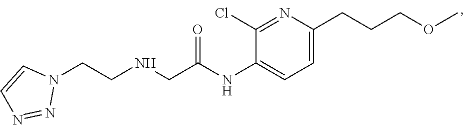 [SMILES: COCCCc1ccc(NC(=O)CNCCn2ccnn2)c(Cl)n1] | (ig) |
| 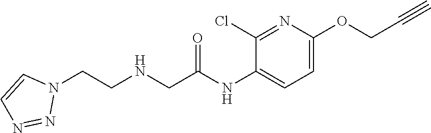 [SMILES: C#CCOc1ccc(NC(=O)CNCCn2ccnn2)c(Cl)n1] | (ih) |
| 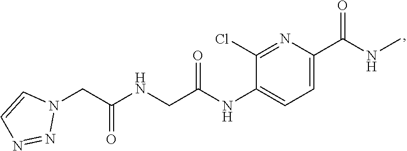 [SMILES: CNC(=O)c1ccc(NC(=O)CNC(=O)Cn2ccnn2)c(Cl)n1] | (ii) |
| 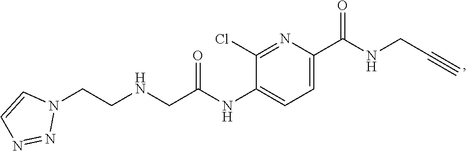 [SMILES: C#CCNC(=O)c1ccc(NC(=O)CNCCn2ccnn2)c(Cl)n1] | (ij) |
| 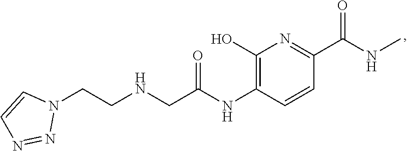 [SMILES: CNC(=O)c1ccc(NC(=O)CNCCn2ccnn2)c(O)n1] | (ik) |
| 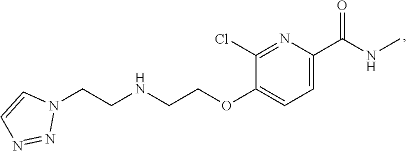 [SMILES: CNC(=O)c1ccc(OCCNCCn2ccnn2)c(Cl)n1] | (il) |
| 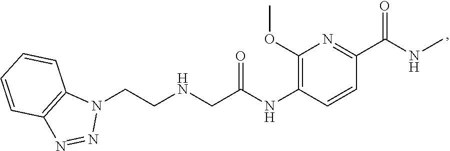 [SMILES: CNC(=O)c1ccc(NC(=O)CNCCn2nnc3ccccc32)c(OC)n1] | (im) |
| 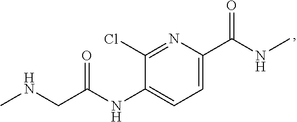 | (in) |
| 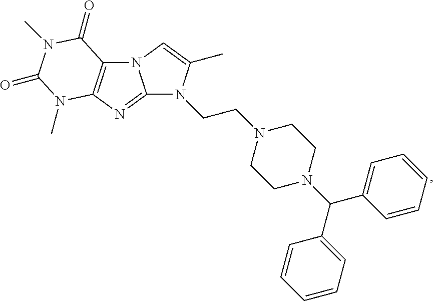 [SMILES: Cc1cn2c3c(=O)n(C)c(=O)n(C)c3nc2n1CCN1CCN(C(c2ccccc2)c2ccccc2)CC1] | (io) |
| 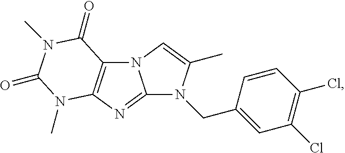 [SMILES: Cc1cn2c3c(=O)n(C)c(=O)n(C)c3nc2n1Cc1ccc(Cl)c(Cl)c1] | (ip) |
| 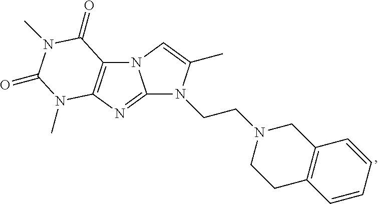 [SMILES: Cc1cn2c3c(=O)n(C)c(=O)n(C)c3nc2n1CCN1CCc2ccccc2C1] | (iq) |
| 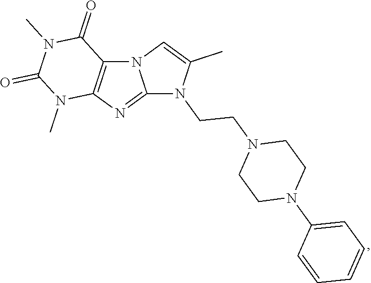 [SMILES: Cc1cn2c3c(=O)n(C)c(=O)n(C)c3nc2n1CCN1CCN(c2ccccc2)CC1] | (ir) |
| 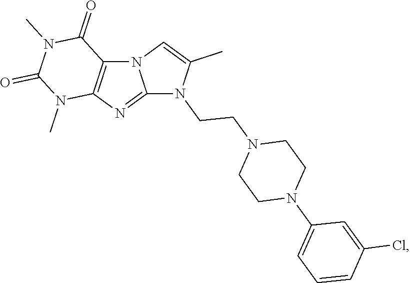 [SMILES: Cc1cn2c3c(=O)n(C)c(=O)n(C)c3nc2n1CCN1CCN(c2cccc(Cl)c2)CC1] | (is) |
| 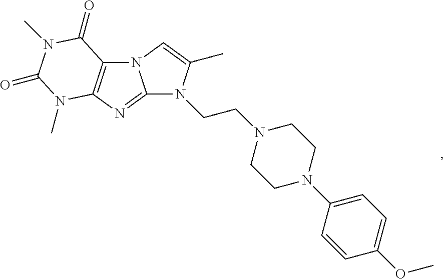 [SMILES: COc1ccc(N2CCN(CCn3c(C)cn4c5c(=O)n(C)c(=O)n(C)c5nc34)CC2)cc1] | (it) |
| 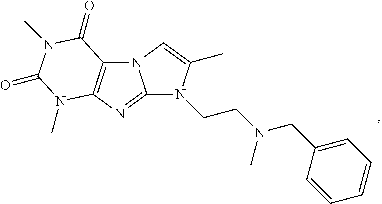 [SMILES: Cc1cn2c3c(=O)n(C)c(=O)n(C)c3nc2n1CCN(C)Cc1ccccc1] | (iu) |
| 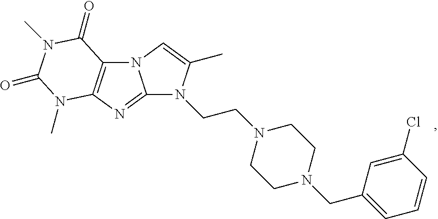 [SMILES: Cc1cn2c3c(=O)n(C)c(=O)n(C)c3nc2n1CCN1CCN(Cc2cccc(Cl)c2)CC1] | (iv) |
| 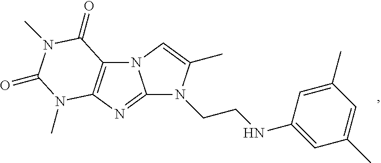 [SMILES: Cc1cc(C)cc(NCCn2c(C)cn3c4c(=O)n(C)c(=O)n(C)c4nc23)c1] | (iw) |
| 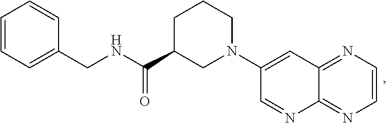 [SMILES: O=C(NCc1ccccc1)[C@H]1CCCN(c2cnc3nccnc3c2)C1] | (ix) |
| 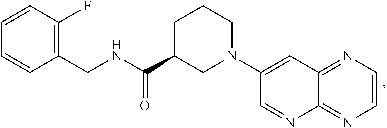 [SMILES: O=C(NCc1ccccc1F)[C@H]1CCCN(c2cnc3nccnc3c2)C1] | (iy) |
| 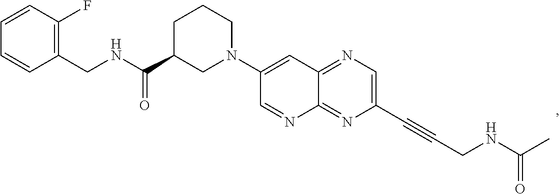 [SMILES: CC(=O)NCC#Cc1cnc2cc(N3CCC[C@H](C(=O)NCc4ccccc4F)C3)cnc2n1] | (iz) |
| 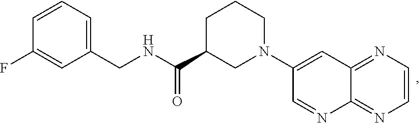 [SMILES: O=C(NCc1cccc(F)c1)[C@H]1CCCN(c2cnc3nccnc3c2)C1] | (ja) |
| 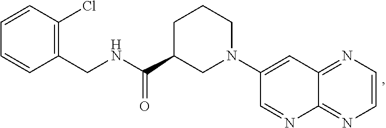 [SMILES: O=C(NCc1ccccc1Cl)[C@H]1CCCN(c2cnc3nccnc3c2)C1] | (jb) |
| 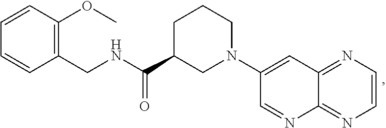 [SMILES: COc1ccccc1CNC(=O)[C@H]1CCCN(c2cnc3nccnc3c2)C1] | (jc) |
| 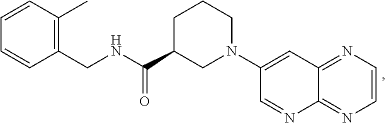 [SMILES: Cc1ccccc1CNC(=O)[C@H]1CCCN(c2cnc3nccnc3c2)C1] | (jd) |
| 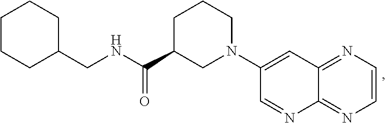 [SMILES: O=C(NCC1CCCCC1)[C@H]1CCCN(c2cnc3nccnc3c2)C1] | (je) |
| 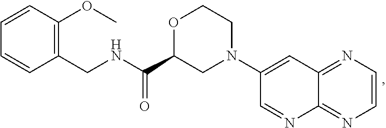 [SMILES: COc1ccccc1CNC(=O)[C@@H]1CN(c2cnc3nccnc3c2)CCO1] | (jf) |
| 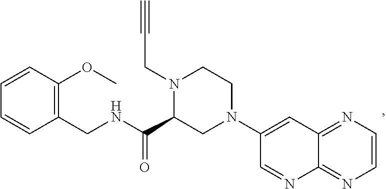 [SMILES: C#CCN1CCN(c2cnc3nccnc3c2)C[C@H]1C(=O)NCc1ccccc1OC] | (jg) |
| 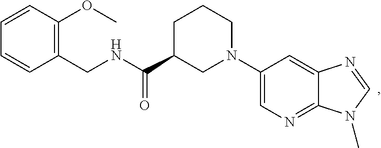 [SMILES: COc1ccccc1CNC(=O)[C@H]1CCCN(c2cnc3c(c2)ncn3C)C1] | (jh) |
| 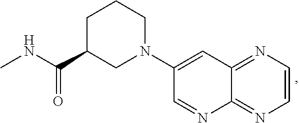 [SMILES: CNC(=O)[C@H]1CCCN(c2cnc3nccnc3c2)C1] | (ji) |
| 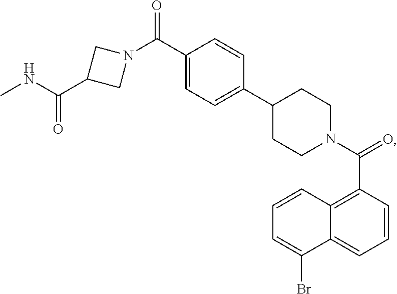 [SMILES: CNC(=O)C1CN(C(=O)c2ccc(C3CCN(C(=O)c4cccc5c(Br)cccc45)CC3)cc2)C1] | (jj) |
| 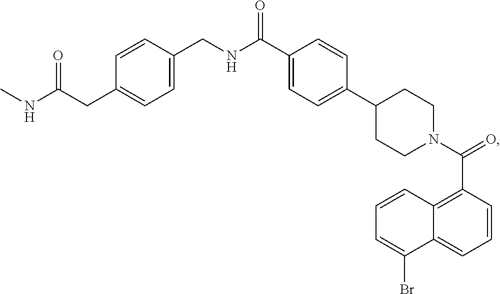 [SMILES: CNC(=O)Cc1ccc(CNC(=O)c2ccc(C3CCN(C(=O)c4cccc5c(Br)cccc45)CC3)cc2)cc1] | (jk) |
| 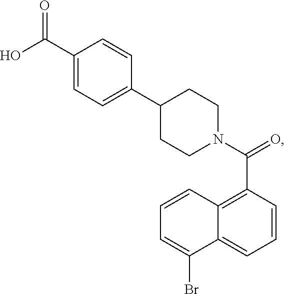 [SMILES: O=C(O)c1ccc(C2CCN(C(=O)c3cccc4c(Br)cccc34)CC2)cc1] | (jl) |
| 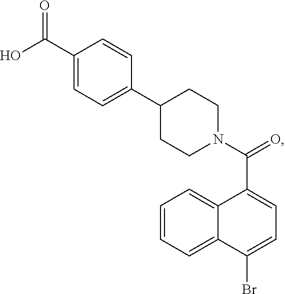 [SMILES: O=C(O)c1ccc(C2CCN(C(=O)c3ccc(Br)c4ccccc34)CC2)cc1] | (jm) |
| 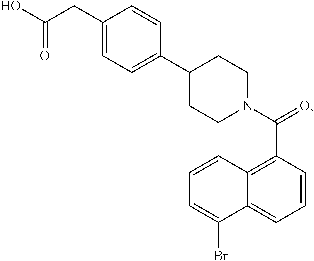 [SMILES: O=C(O)Cc1ccc(C2CCN(C(=O)c3cccc4c(Br)cccc34)CC2)cc1] | (jn) |
| 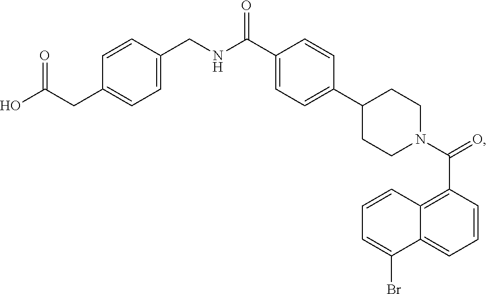 [SMILES: O=C(O)Cc1ccc(CNC(=O)c2ccc(C3CCN(C(=O)c4cccc5c(Br)cccc45)CC3)cc2)cc1] | (jo) |
| 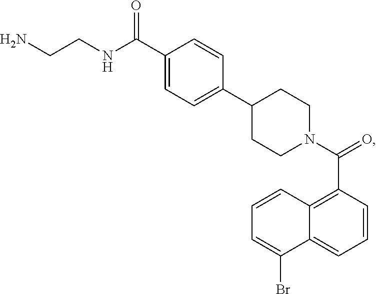 [SMILES: NCCNC(=O)c1ccc(C2CCN(C(=O)c3cccc4c(Br)cccc34)CC2)cc1] | (jp) |
| 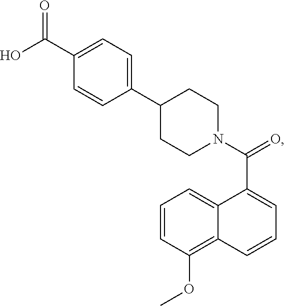 [SMILES: COc1cccc2c(C(=O)N3CCC(c4ccc(C(=O)O)cc4)CC3)cccc12] | (jq) |
| 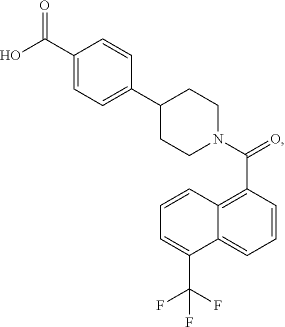 [SMILES: O=C(O)c1ccc(C2CCN(C(=O)c3cccc4c(C(F)(F)F)cccc34)CC2)cc1] | (jr) |
| 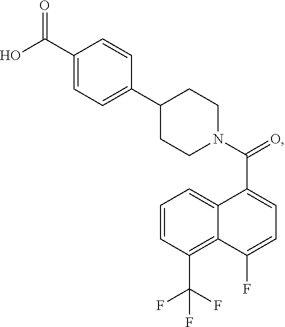 [SMILES: O=C(O)c1ccc(C2CCN(C(=O)c3ccc(F)c4c(C(F)(F)F)cccc34)CC2)cc1] | (js) |
| 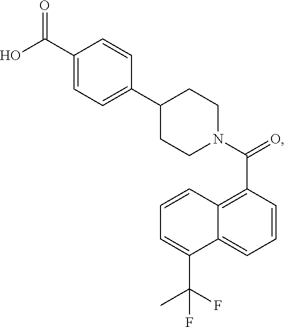 [SMILES: CC(F)(F)c1cccc2c(C(=O)N3CCC(c4ccc(C(=O)O)cc4)CC3)cccc12] | (jt) |
| 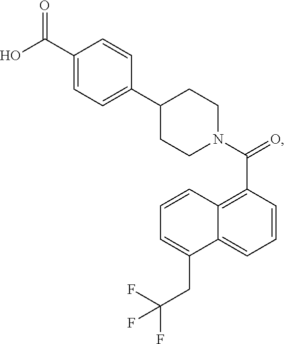 [SMILES: O=C(O)c1ccc(C2CCN(C(=O)c3cccc4c(CC(F)(F)F)cccc34)CC2)cc1] | (ju) |
| 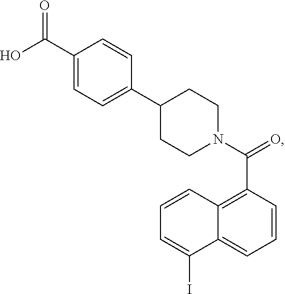 [SMILES: O=C(O)c1ccc(C2CCN(C(=O)c3cccc4c(I)cccc34)CC2)cc1] | (jv) |
| 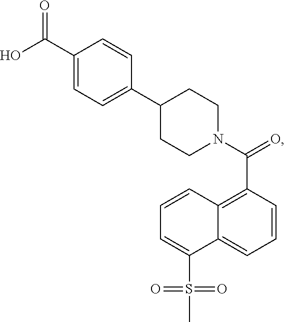 [SMILES: CS(=O)(=O)c1cccc2c(C(=O)N3CCC(c4ccc(C(=O)O)cc4)CC3)cccc12] | (jw) |
| 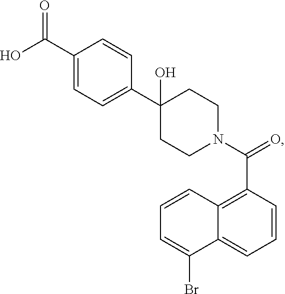 [SMILES: O=C(O)c1ccc(C2(O)CCN(C(=O)c3cccc4c(Br)cccc34)CC2)cc1] | (jx) |
| 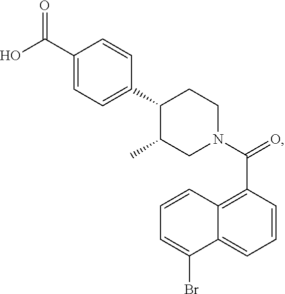 [SMILES: C[C@H]1CN(C(=O)c2cccc3c(Br)cccc23)CC[C@H]1c1ccc(C(=O)O)cc1] | (jy) |
| 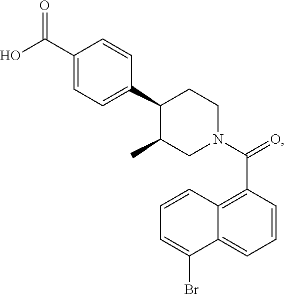 [SMILES: C[C@@H]1CN(C(=O)c2cccc3c(Br)cccc23)CC[C@@H]1c1ccc(C(=O)O)cc1] | (jz) |
| 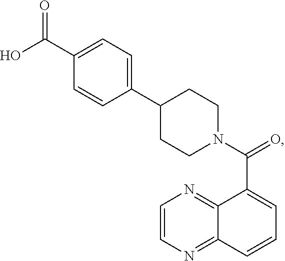 [SMILES: O=C(O)c1ccc(C2CCN(C(=O)c3cccc4nccnc34)CC2)cc1] | (ka) |
| 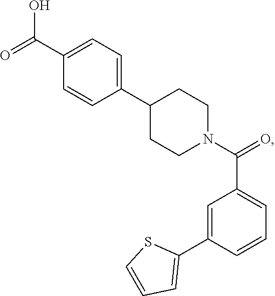 [SMILES: O=C(O)c1ccc(C2CCN(C(=O)c3cccc(-c4cccs4)c3)CC2)cc1] | (kb) |
| 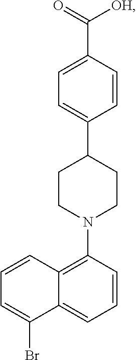 [SMILES: O=C(O)c1ccc(C2CCN(c3cccc4c(Br)cccc34)CC2)cc1] | (kc) |
| 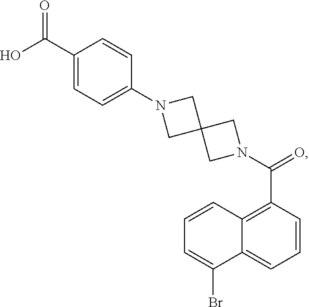 [SMILES: O=C(O)c1ccc(N2CC3(CN(C(=O)c4cccc5c(Br)cccc45)C3)C2)cc1] | (kd) |
| 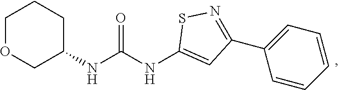 [SMILES: O=C(Nc1cc(-c2ccccc2)ns1)N[C@H]1CCCOC1] | (kw) |
| 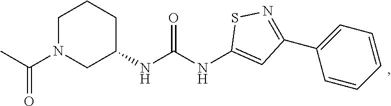 [SMILES: CC(=O)N1CCC[C@H](NC(=O)Nc2cc(-c3ccccc3)ns2)C1] | (kx) |
| 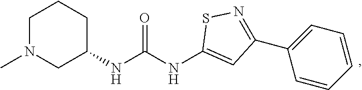 [SMILES: CN1CCC[C@H](NC(=O)Nc2cc(-c3ccccc3)ns2)C1] | (ky) |
| 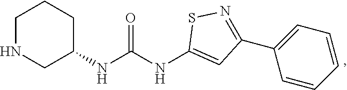 [SMILES: O=C(Nc1cc(-c2ccccc2)ns1)N[C@H]1CCCNC1] | (kz) |
| 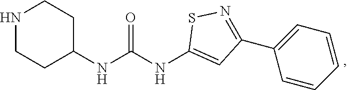 [SMILES: O=C(Nc1cc(-c2ccccc2)ns1)NC1CCNCC1] | (la) |
| 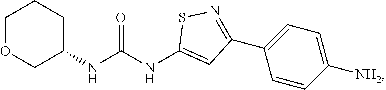 [SMILES: Nc1ccc(-c2cc(NC(=O)N[C@H]3CCCOC3)sn2)cc1] | (lb) |
| 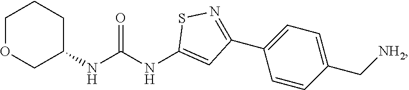 [SMILES: NCc1ccc(-c2cc(NC(=O)N[C@H]3CCCOC3)sn2)cc1] | (lc) |
| 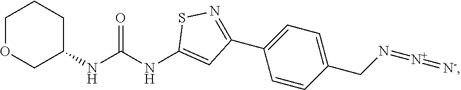 | (ld) |
| 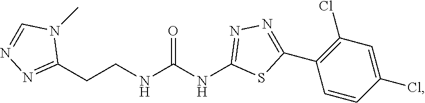 [SMILES: Cn1cnnc1CCNC(=O)Nc1nnc(-c2ccc(Cl)cc2Cl)s1] | (le) |
| 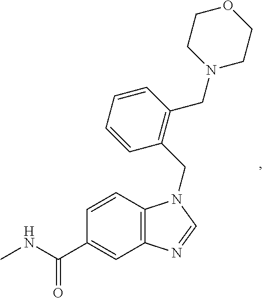 [SMILES: CNC(=O)c1ccc2c(c1)ncn2Cc1ccccc1CN1CCOCC1] | (lf) |
| 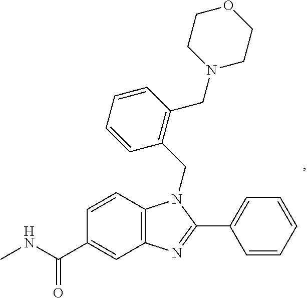 [SMILES: CNC(=O)c1ccc2c(c1)nc(-c1ccccc1)n2Cc1ccccc1CN1CCOCC1] | (lg) |
| 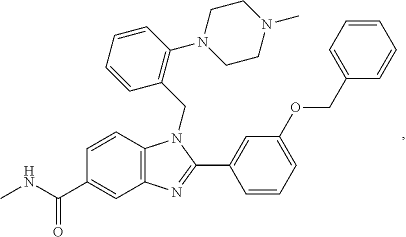 [SMILES: CNC(=O)c1ccc2c(c1)nc(-c1cccc(OCc3ccccc3)c1)n2Cc1ccccc1N1CCN(C)CC1] | (lh) |
| 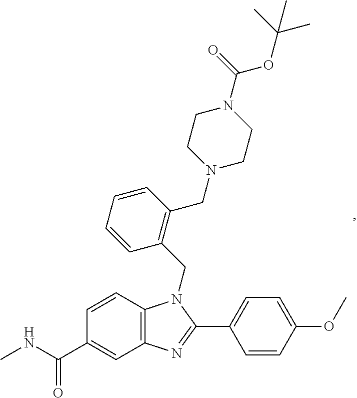 | (li) |
| 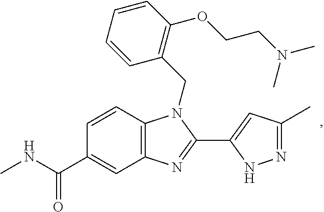 | (lj) |
| 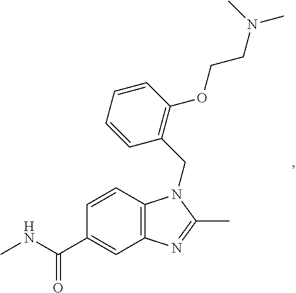 | (lk) |
| 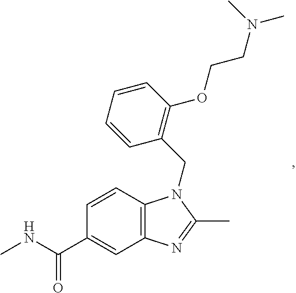 | (ll) |
| 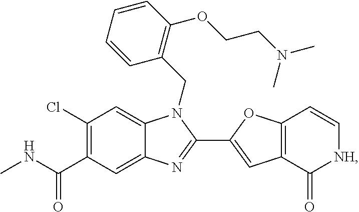 [SMILES: CNC(=O)c1cc2nc(-c3cc4c(=O)[nH]ccc4o3)n(Cc3ccccc3OCCN(C)C)c2cc1Cl] | (lm) |
| 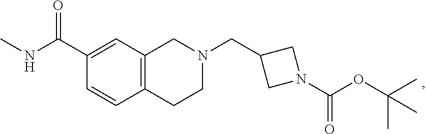 [SMILES: CNC(=O)c1ccc2c(c1)CN(CC1CN(C(=O)OC(C)(C)C)C1)CC2] | (ln) |
| 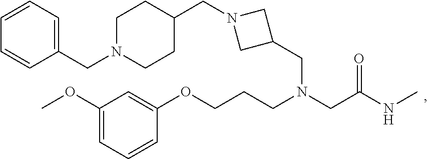 [SMILES: CNC(=O)CN(CCCOc1cccc(OC)c1)CC1CN(CC2CCN(Cc3ccccc3)CC2)C1] | (lo) |
| 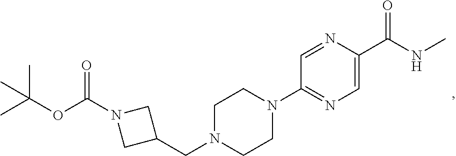 [SMILES: CNC(=O)c1cnc(N2CCN(CC3CN(C(=O)OC(C)(C)C)C3)CC2)cn1] | (lp) |
| 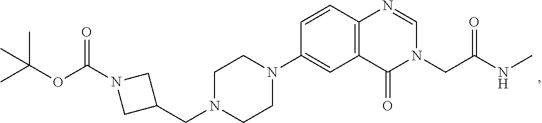 [SMILES: CNC(=O)Cn1cnc2ccc(N3CCN(CC4CN(C(=O)OC(C)(C)C)C4)CC3)cc2c1=O] | (lq) |
| 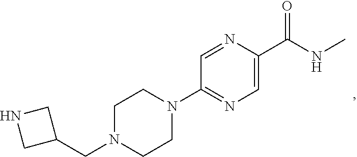 [SMILES: CNC(=O)c1cnc(N2CCN(CC3CNC3)CC2)cn1] | (lr) |
| 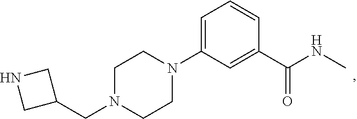 [SMILES: CNC(=O)c1cccc(N2CCN(CC3CNC3)CC2)c1] | (ls) |
| 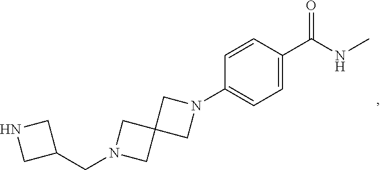 [SMILES: CNC(=O)c1ccc(N2CC3(CN(CC4CNC4)C3)C2)cc1] | (lt) |
| 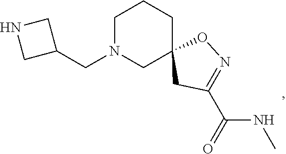 [SMILES: CNC(=O)C1=NO[C@]2(CCCN(CC3CNC3)C2)C1] | (lu) |
| 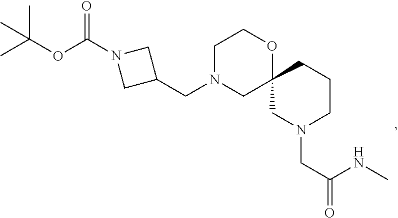 [SMILES: CNC(=O)CN1CCC[C@@]2(C1)CN(CC1CN(C(=O)OC(C)(C)C)C1)CCO2] | (lv) |
| 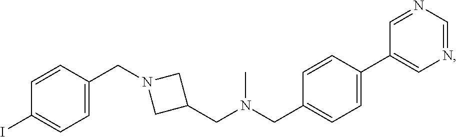 [SMILES: CN(Cc1ccc(-c2cncnc2)cc1)CC1CN(Cc2ccc(I)cc2)C1] | (lw) |
| 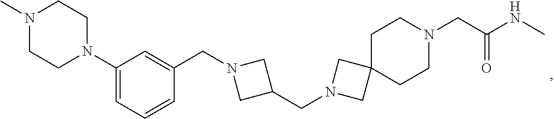 | (lx) |
| 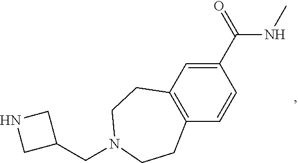 [SMILES: CNC(=O)c1ccc2c(c1)CCN(CC1CNC1)CC2] | (ly) |
| 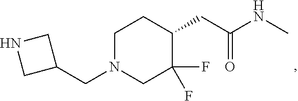 [SMILES: CNC(=O)C[C@H]1CCN(CC2CNC2)CC1(F)F] | (lz) |
| 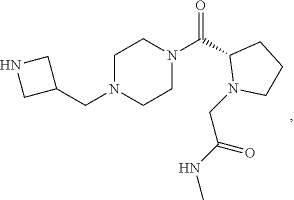 [SMILES: CNC(=O)CN1CCC[C@H]1C(=O)N1CCN(CC2CNC2)CC1] | (ma) |
| 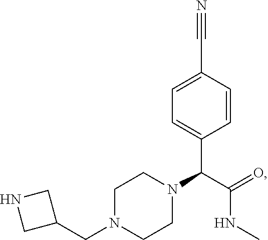 [SMILES: CNC(=O)[C@H](c1ccc(C#N)cc1)N1CCN(CC2CNC2)CC1] | (mb) |
| 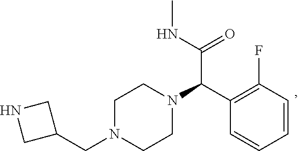 [SMILES: CNC(=O)[C@@H](c1ccccc1F)N1CCN(CC2CNC2)CC1] | (mc) |
| 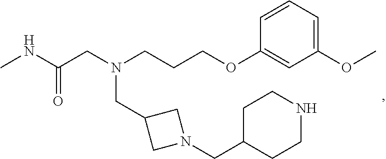 | (md) |
| 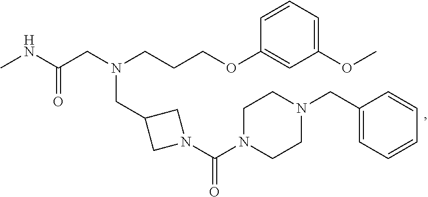 [SMILES: CNC(=O)CN(CCCOc1cccc(OC)c1)CC1CN(C(=O)N2CCN(Cc3ccccc3)CC2)C1] | (me) |
| 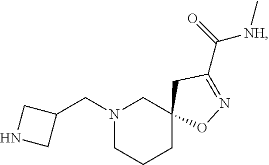 [SMILES: CNC(=O)C1=NO[C@@]2(CCCN(CC3CNC3)C2)C1] | (mf) |
| 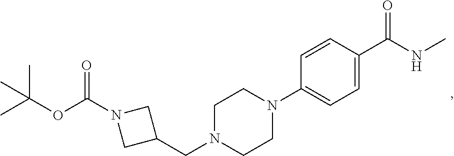 [SMILES: CNC(=O)c1ccc(N2CCN(CC3CN(C(=O)OC(C)(C)C)C3)CC2)cc1] | (mg) |
| 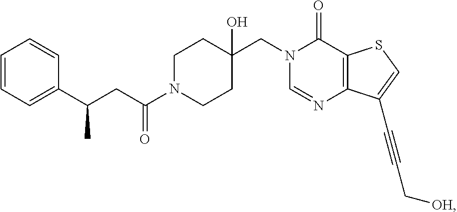 [SMILES: C[C@H](CC(=O)N1CCC(O)(Cn2cnc3c(C#CCO)csc3c2=O)CC1)c1ccccc1] | (mh) |
| 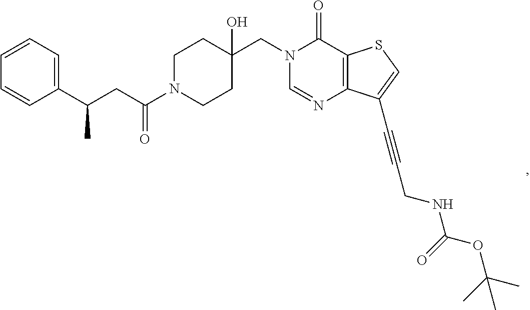 [SMILES: C[C@H](CC(=O)N1CCC(O)(Cn2cnc3c(C#CCNC(=O)OC(C)(C)C)csc3c2=O)CC1)c1ccccc1] | (mi) |
| 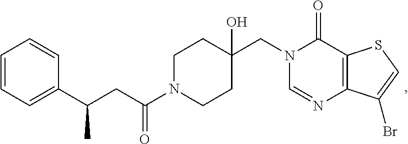 [SMILES: C[C@H](CC(=O)N1CCC(O)(Cn2cnc3c(Br)csc3c2=O)CC1)c1ccccc1] | (mj) |
| 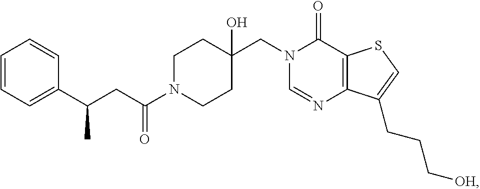 [SMILES: C[C@H](CC(=O)N1CCC(O)(Cn2cnc3c(CCCO)csc3c2=O)CC1)c1ccccc1] | (mk) |
| 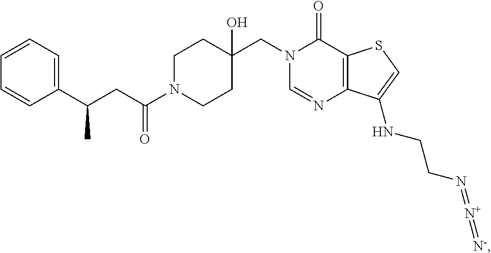 | (ml) |
| 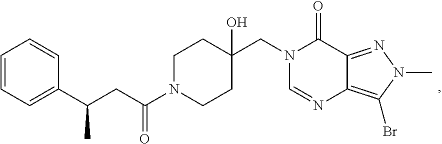 [SMILES: C[C@H](CC(=O)N1CCC(O)(Cn2cnc3c(Br)n(C)nc3c2=O)CC1)c1ccccc1] | (mm) |
| 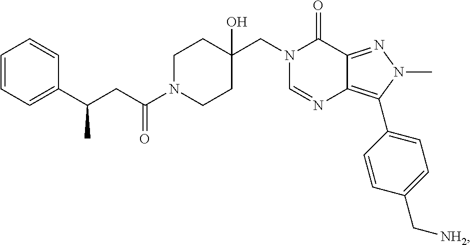 [SMILES: C[C@H](CC(=O)N1CCC(O)(Cn2cnc3c(-c4ccc(CN)cc4)n(C)nc3c2=O)CC1)c1ccccc1] | (mn) |
| 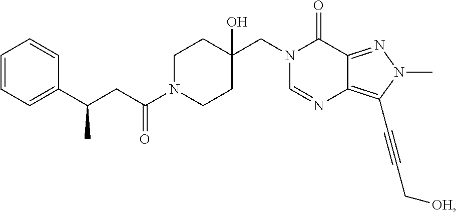 [SMILES: C[C@H](CC(=O)N1CCC(O)(Cn2cnc3c(C#CCO)n(C)nc3c2=O)CC1)c1ccccc1] | (mo) |
| 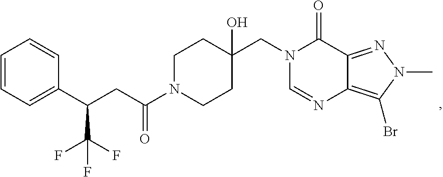 [SMILES: Cn1nc2c(=O)n(CC3(O)CCN(C(=O)C[C@H](c4ccccc4)C(F)(F)F)CC3)cnc2c1Br] | (mp) |
| 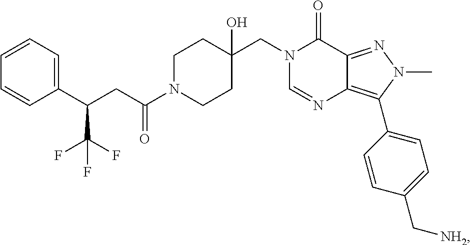 [SMILES: Cn1nc2c(=O)n(CC3(O)CCN(C(=O)C[C@H](c4ccccc4)C(F)(F)F)CC3)cnc2c1-c1ccc(CN)cc1] | (mq) |
| 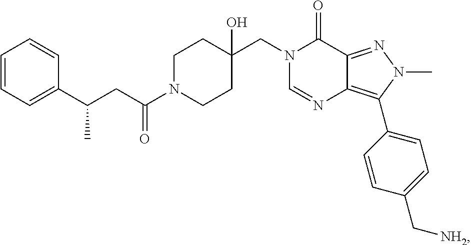 [SMILES: C[C@@H](CC(=O)N1CCC(O)(Cn2cnc3c(-c4ccc(CN)cc4)n(C)nc3c2=O)CC1)c1ccccc1] | (mr) |
| 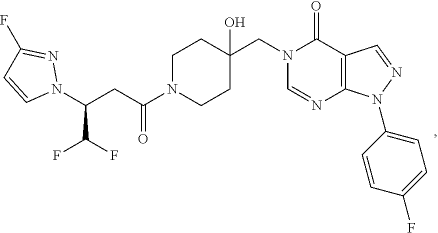 [SMILES: O=C(C[C@@H](C(F)F)n1ccc(F)n1)N1CCC(O)(Cn2cnc3c(cnn3-c3ccc(F)cc3)c2=O)CC1] | (ms) |
| 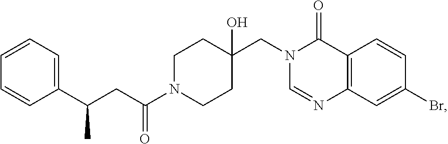 [SMILES: C[C@H](CC(=O)N1CCC(O)(Cn2cnc3cc(Br)ccc3c2=O)CC1)c1ccccc1] | (mt) |
| 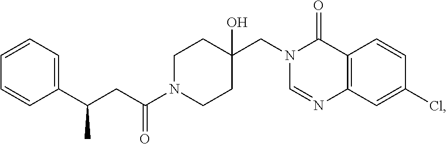 [SMILES: C[C@H](CC(=O)N1CCC(O)(Cn2cnc3cc(Cl)ccc3c2=O)CC1)c1ccccc1] | (mu) |
| 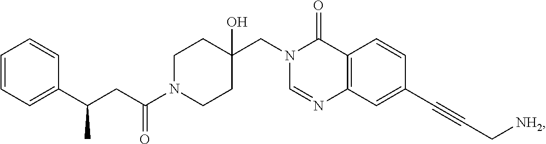 [SMILES: C[C@H](CC(=O)N1CCC(O)(Cn2cnc3cc(C#CCN)ccc3c2=O)CC1)c1ccccc1] | (mv) |
| 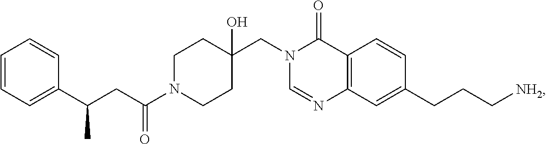 [SMILES: C[C@H](CC(=O)N1CCC(O)(Cn2cnc3cc(CCCN)ccc3c2=O)CC1)c1ccccc1] | (mw) |
| 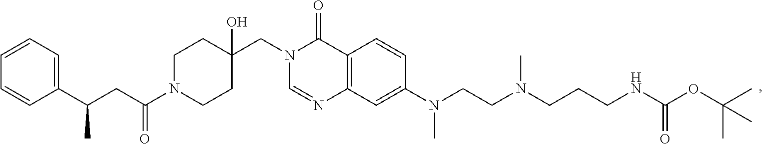 [SMILES: C[C@H](CC(=O)N1CCC(O)(Cn2cnc3cc(N(C)CCN(C)CCCNC(=O)OC(C)(C)C)ccc3c2=O)CC1)c1ccccc1] | (mx) |
| 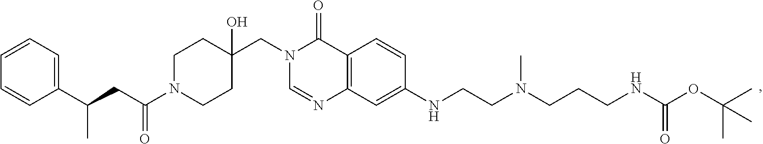 [SMILES: C[C@H](CC(=O)N1CCC(O)(Cn2cnc3cc(NCCN(C)CCCNC(=O)OC(C)(C)C)ccc3c2=O)CC1)c1ccccc1] | (my) |
| 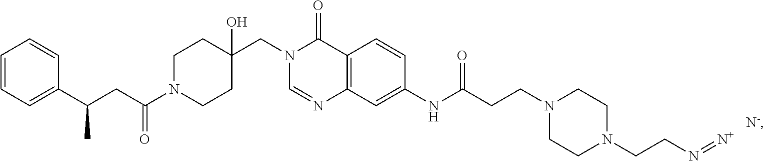 | (mz) |
| 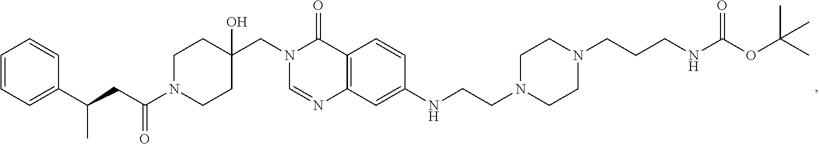 [SMILES: C[C@H](CC(=O)N1CCC(O)(Cn2cnc3cc(NCCN4CCN(CCCNC(=O)OC(C)(C)C)CC4)ccc3c2=O)CC1)c1ccccc1] | (na) |
| 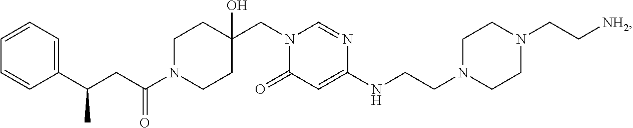 [SMILES: C[C@H](CC(=O)N1CCC(O)(Cn2cnc(NCCN3CCN(CCN)CC3)cc2=O)CC1)c1ccccc1] | (nb) |
| 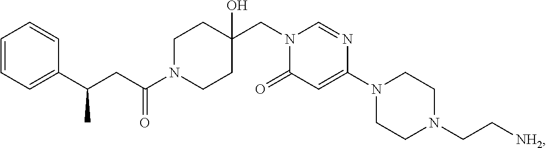 [SMILES: C[C@H](CC(=O)N1CCC(O)(Cn2cnc(N3CCN(CCN)CC3)cc2=O)CC1)c1ccccc1] | (nc) |
| 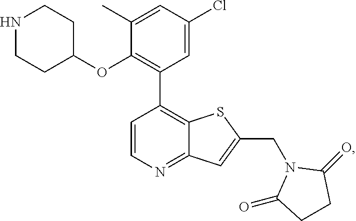 [SMILES: Cc1cc(Cl)cc(-c2ccnc3cc(CN4C(=O)CCC4=O)sc23)c1OC1CCNCC1] | (nd) |
| 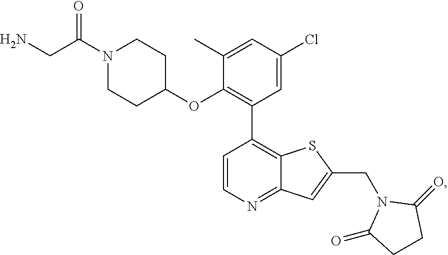 [SMILES: Cc1cc(Cl)cc(-c2ccnc3cc(CN4C(=O)CCC4=O)sc23)c1OC1CCN(C(=O)CN)CC1] | (ne) |
| 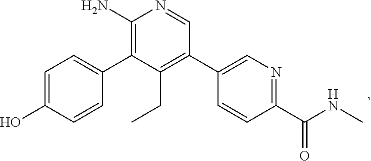 [SMILES: CCc1c(-c2ccc(C(=O)NC)nc2)cnc(N)c1-c1ccc(O)cc1] | (nf) |
| 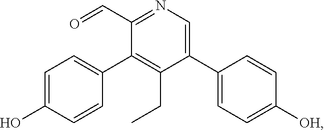 [SMILES: CCc1c(-c2ccc(O)cc2)cnc(C=O)c1-c1ccc(O)cc1] | (ng) |
| 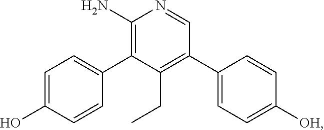 [SMILES: CCc1c(-c2ccc(O)cc2)cnc(N)c1-c1ccc(O)cc1] | (nh) |
| 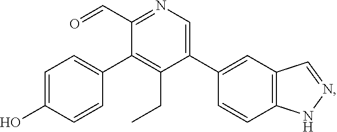 [SMILES: CCc1c(-c2ccc3[nH]ncc3c2)cnc(C=O)c1-c1ccc(O)cc1] | (ni) |
| 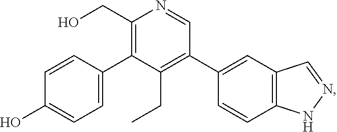 [SMILES: CCc1c(-c2ccc3[nH]ncc3c2)cnc(CO)c1-c1ccc(O)cc1] | (nj) |
| 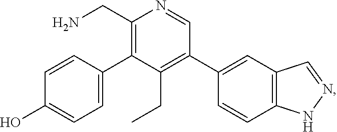 [SMILES: CCc1c(-c2ccc3[nH]ncc3c2)cnc(CN)c1-c1ccc(O)cc1] | (nk) |
| 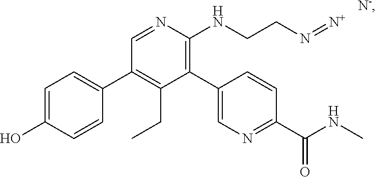 | (nl) |
| 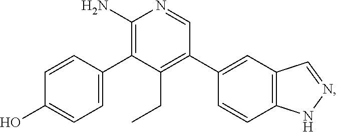 [SMILES: CCc1c(-c2ccc3[nH]ncc3c2)cnc(N)c1-c1ccc(O)cc1] | (nm) |
| 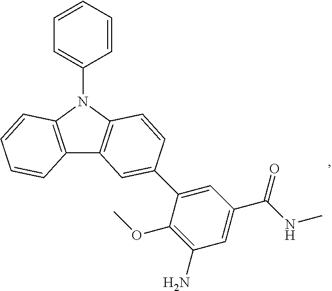 [SMILES: CNC(=O)c1cc(N)c(OC)c(-c2ccc3c(c2)c2ccccc2n3-c2ccccc2)c1] | (nn) |
| 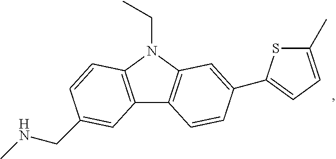 | (no) |
| 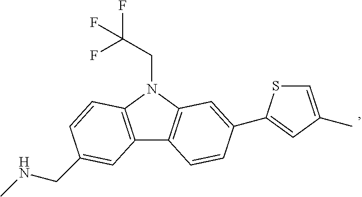 | (np) |
| 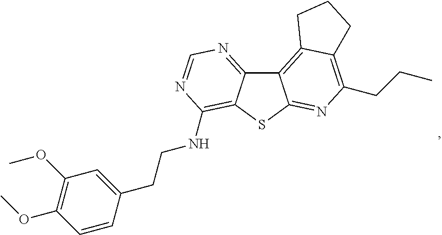 [SMILES: CCCc1nc2sc3c(NCCc4ccc(OC)c(OC)c4)ncnc3c2c2c1CCC2] | (nq) |
| 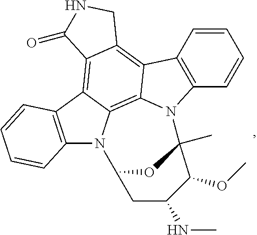 [SMILES: CN[C@@H]1C[C@@H]2O[C@@](C)([C@@H]1OC)n1c3ccccc3c3c4c(c5c6ccccc6n2c5c31)C(=O)NC4] | (nr) |
| 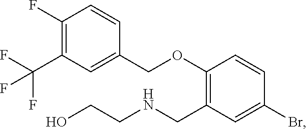 [SMILES: OCCNCc1cc(Br)ccc1OCc1ccc(F)c(C(F)(F)F)c1] | (ns) |
| 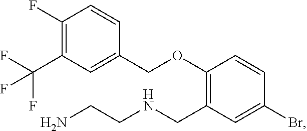 [SMILES: NCCNCc1cc(Br)ccc1OCc1ccc(F)c(C(F)(F)F)c1] | (nt) |
| 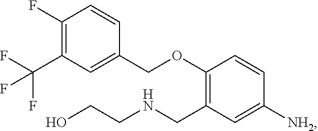 [SMILES: Nc1ccc(OCc2ccc(F)c(C(F)(F)F)c2)c(CNCCO)c1] | (nu) |
| 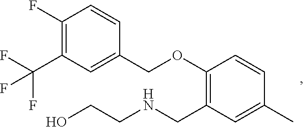 [SMILES: Cc1ccc(OCc2ccc(F)c(C(F)(F)F)c2)c(CNCCO)c1] | (nv) |
| 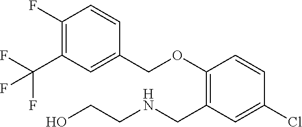 [SMILES: OCCNCc1cc(Cl)ccc1OCc1ccc(F)c(C(F)(F)F)c1] | (nw) |
| 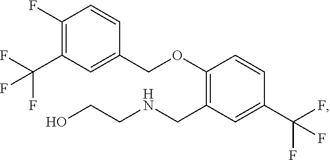 [SMILES: OCCNCc1cc(C(F)(F)F)ccc1OCc1ccc(F)c(C(F)(F)F)c1] | (nx) |
| 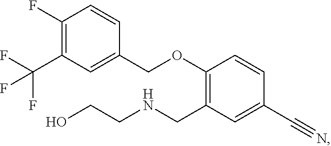 [SMILES: N#Cc1ccc(OCc2ccc(F)c(C(F)(F)F)c2)c(CNCCO)c1] | (ny) |
| 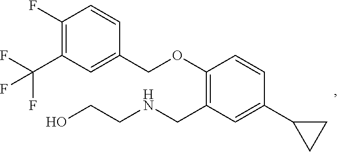 [SMILES: OCCNCc1cc(C2CC2)ccc1OCc1ccc(F)c(C(F)(F)F)c1] | (nz) |
| 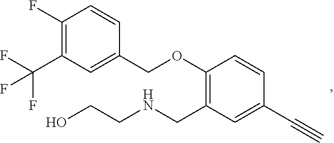 [SMILES: C#Cc1ccc(OCc2ccc(F)c(C(F)(F)F)c2)c(CNCCO)c1] | (oa) |
| 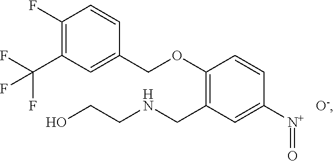 | (ob) |
| 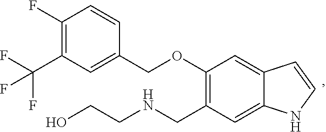 [SMILES: OCCNCc1cc2[nH]ccc2cc1OCc1ccc(F)c(C(F)(F)F)c1] | (oc) |
| 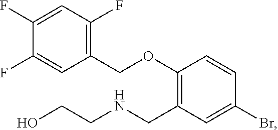 [SMILES: OCCNCc1cc(Br)ccc1OCc1cc(F)c(F)cc1F] | (od) |
| 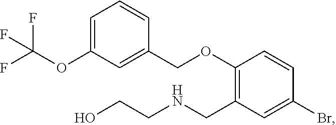 [SMILES: OCCNCc1cc(Br)ccc1OCc1cccc(OC(F)(F)F)c1] | (oe) |
| 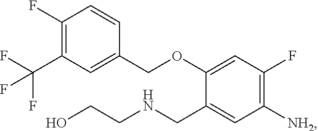 [SMILES: Nc1cc(CNCCO)c(OCc2ccc(F)c(C(F)(F)F)c2)cc1F] | (of) |
| 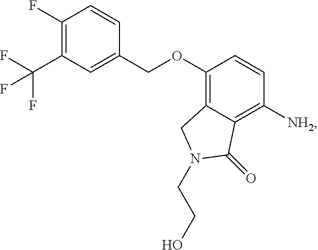 [SMILES: Nc1ccc(OCc2ccc(F)c(C(F)(F)F)c2)c2c1C(=O)N(CCO)C2] | (og) |
| 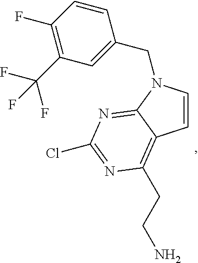 [SMILES: NCCc1nc(Cl)nc2c1ccn2Cc1ccc(F)c(C(F)(F)F)c1] | (oh) |
| 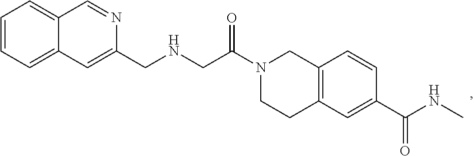 [SMILES: CNC(=O)c1ccc2c(c1)CCN(C(=O)CNCc1cc3ccccc3cn1)C2] | (oi) |
| 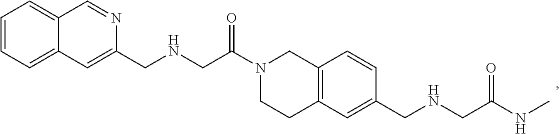 [SMILES: CNC(=O)CNCc1ccc2c(c1)CCN(C(=O)CNCc1cc3ccccc3cn1)C2] | (oj) |
| 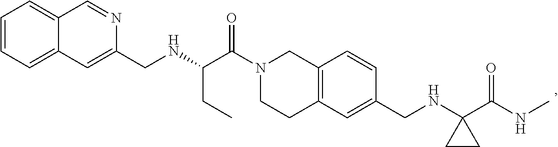 [SMILES: CC[C@H](NCc1cc2ccccc2cn1)C(=O)N1CCc2cc(CNC3(C(=O)NC)CC3)ccc2C1] | (ok) |
| 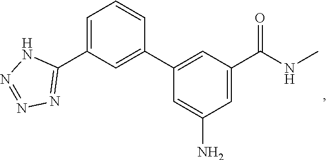 [SMILES: CNC(=O)c1cc(N)cc(-c2cccc(-c3nnn[nH]3)c2)c1] | (ol) |
| 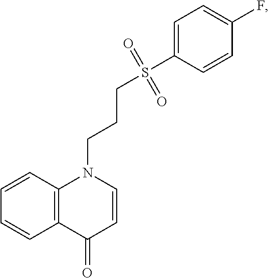 [SMILES: O=c1ccn(CCCS(=O)(=O)c2ccc(F)cc2)c2ccccc12] | (om) |
| 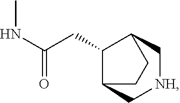 | (on) |
| 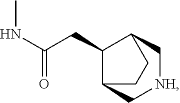 | (oo) |
| 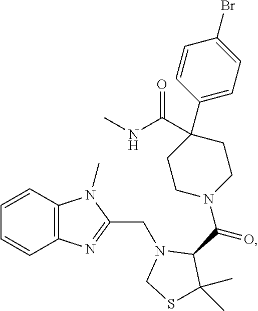 [SMILES: CNC(=O)C1(c2ccc(Br)cc2)CCN(C(=O)[C@@H]2N(Cc3nc4ccccc4n3C)CSC2(C)C)CC1] | (op) |
| 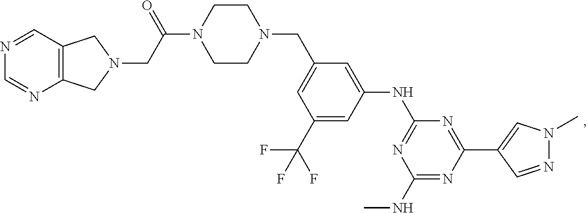 [SMILES: CNc1nc(Nc2cc(CN3CCN(C(=O)CN4Cc5cncnc5C4)CC3)cc(C(F)(F)F)c2)nc(-c2cnn(C)c2)n1] | (oq) |
| 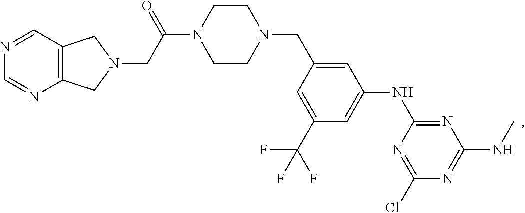 [SMILES: CNc1nc(Cl)nc(Nc2cc(CN3CCN(C(=O)CN4Cc5cncnc5C4)CC3)cc(C(F)(F)F)c2)n1] | (or) |
| 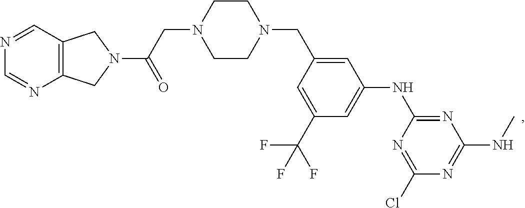 [SMILES: CNc1nc(Cl)nc(Nc2cc(CN3CCN(CC(=O)N4Cc5cncnc5C4)CC3)cc(C(F)(F)F)c2)n1] | (os) |
| 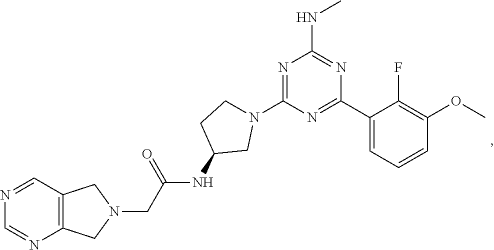 [SMILES: CNc1nc(-c2cccc(OC)c2F)nc(N2CC[C@H](NC(=O)CN3Cc4cncnc4C3)C2)n1] | (ot) |
| 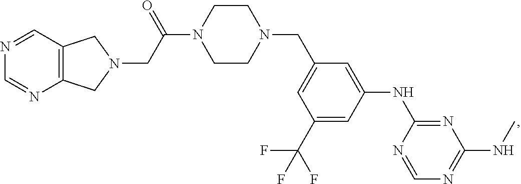 [SMILES: CNc1ncnc(Nc2cc(CN3CCN(C(=O)CN4Cc5cncnc5C4)CC3)cc(C(F)(F)F)c2)n1] | (ou) |
| 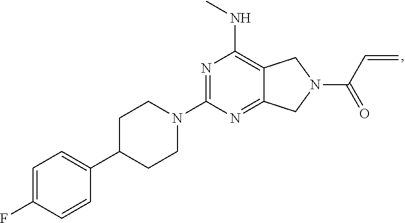 [SMILES: C=CC(=O)N1Cc2nc(N3CCC(c4ccc(F)cc4)CC3)nc(NC)c2C1] | (ov) |
| 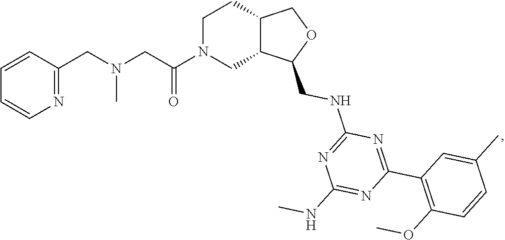 [SMILES: CNc1nc(NC[C@@H]2OC[C@H]3CCN(C(=O)CN(C)Cc4ccccn4)C[C@H]32)nc(-c2cc(C)ccc2OC)n1] | (ow) |
| 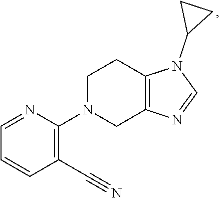 [SMILES: N#Cc1cccnc1N1CCc2c(ncn2C2CC2)C1] | (ox) |
| 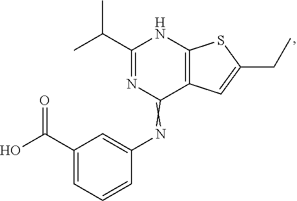 [SMILES: CCc1cc2c(=Nc3cccc(C(=O)O)c3)nc(C(C)C)[nH]c2s1] | (oy) |
| 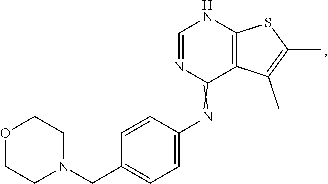 [SMILES: Cc1sc2[nH]cnc(=Nc3ccc(CN4CCOCC4)cc3)c2c1C] | (oz) |
| 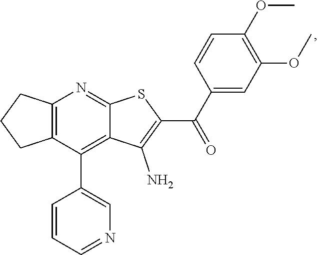 [SMILES: COc1ccc(C(=O)c2sc3nc4c(c(-c5cccnc5)c3c2N)CCC4)cc1OC] | (pa) |
| 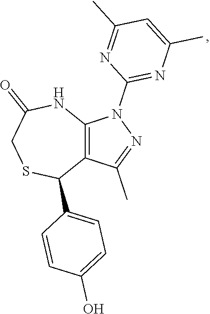 [SMILES: Cc1cc(C)nc(-n2nc(C)c3c2NC(=O)CS[C@@H]3c2ccc(O)cc2)n1] | (pb) |
| 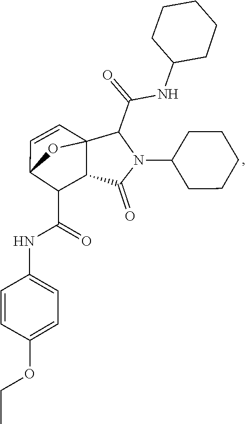 | (pc) |
| 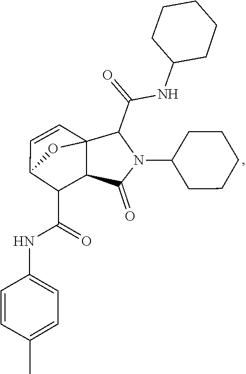 | (pd) |
| 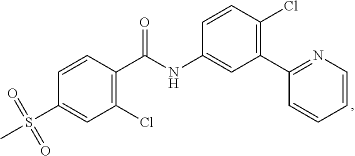 [SMILES: CS(=O)(=O)c1ccc(C(=O)Nc2ccc(Cl)c(-c3ccccn3)c2)c(Cl)c1] | (pe) |
| 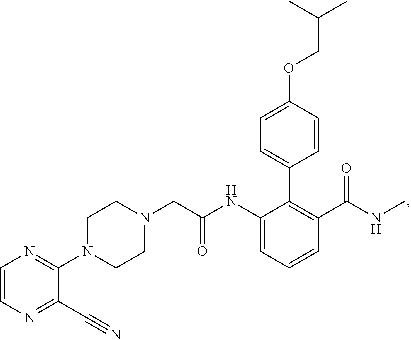 [SMILES: CNC(=O)c1cccc(NC(=O)CN2CCN(c3nccnc3C#N)CC2)c1-c1ccc(OCC(C)C)cc1] | (pf) |
| 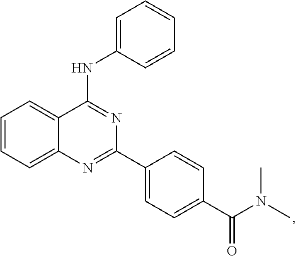 [SMILES: CN(C)C(=O)c1ccc(-c2nc(Nc3ccccc3)c3ccccc3n2)cc1] | (pg) |
| 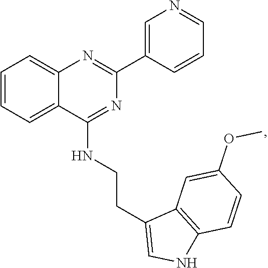 [SMILES: COc1ccc2[nH]cc(CCNc3nc(-c4cccnc4)nc4ccccc34)c2c1] | (ph) |
| 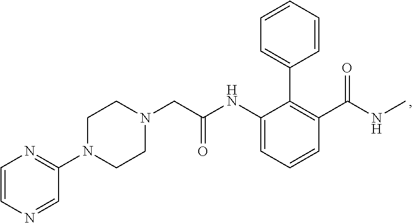 [SMILES: CNC(=O)c1cccc(NC(=O)CN2CCN(c3cnccn3)CC2)c1-c1ccccc1] | (pi) |
| 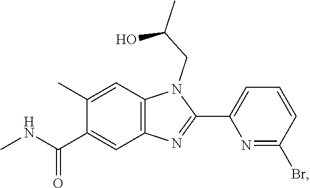 [SMILES: CNC(=O)c1cc2nc(-c3cccc(Br)n3)n(C[C@H](C)O)c2cc1C] | (pj) |
| 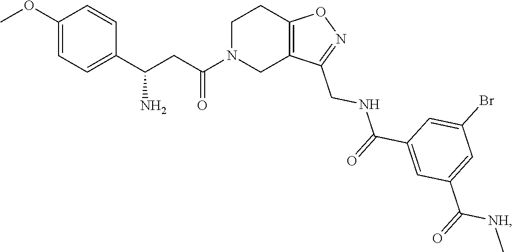 | (pk) |
| 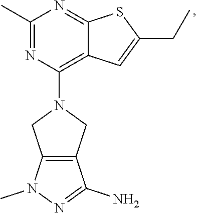 [SMILES: CCc1cc2c(N3Cc4c(N)nn(C)c4C3)nc(C)nc2s1] | (pl) |
| 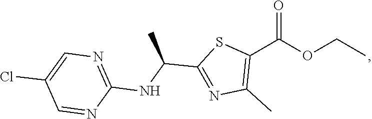 [SMILES: CCOC(=O)c1sc([C@H](C)Nc2ncc(Cl)cn2)nc1C] | (pm) |
| 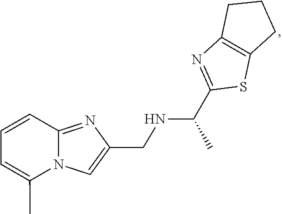 [SMILES: Cc1cccc2nc(CN[C@@H](C)c3nc4c(s3)CCC4)cn12] | (pn) |
| 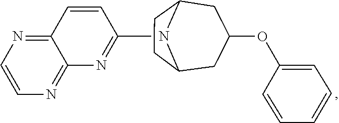 [SMILES: c1ccc(OC2CC3CCC(C2)N3c2ccc3nccnc3n2)cc1] | (po) |
| 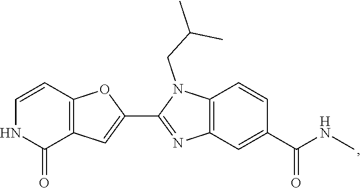 [SMILES: CNC(=O)c1ccc2c(c1)nc(-c1cc3c(=O)[nH]ccc3o1)n2CC(C)C] | (pp) |
| 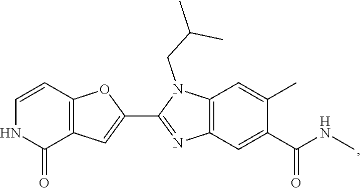 [SMILES: CNC(=O)c1cc2nc(-c3cc4c(=O)[nH]ccc4o3)n(CC(C)C)c2cc1C] | (pq) |
| 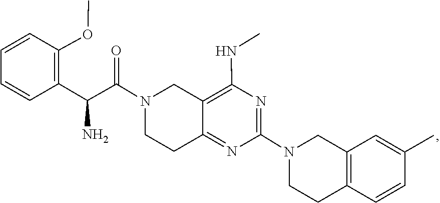 [SMILES: CNc1nc(N2CCc3ccc(C)cc3C2)nc2c1CN(C(=O)[C@@H](N)c1ccccc1OC)CC2] | (pr) |
| 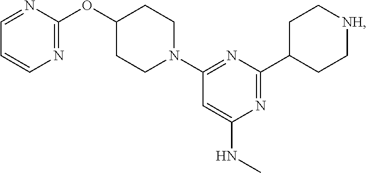 [SMILES: CNc1cc(N2CCC(Oc3ncccn3)CC2)nc(C2CCNCC2)n1] | (ps) |
| 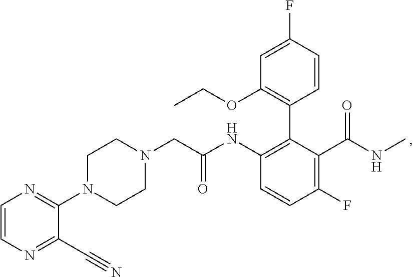 [SMILES: CCOc1cc(F)ccc1-c1c(NC(=O)CN2CCN(c3nccnc3C#N)CC2)ccc(F)c1C(=O)NC] | (pt) |
| 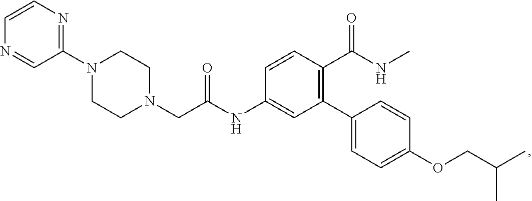 [SMILES: CNC(=O)c1ccc(NC(=O)CN2CCN(c3cnccn3)CC2)cc1-c1ccc(OCC(C)C)cc1] | (pu) |
| 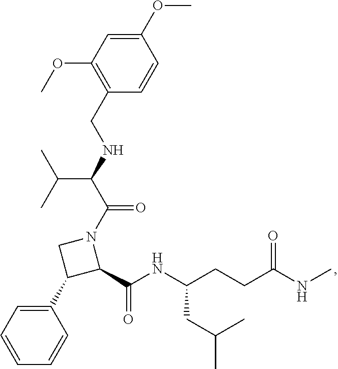 [SMILES: CNC(=O)CC[C@H](CC(C)C)NC(=O)[C@H]1[C@H](c2ccccc2)CN1C(=O)[C@H](NCc1ccc(OC)cc1OC)C(C)C] | (pv) |
| 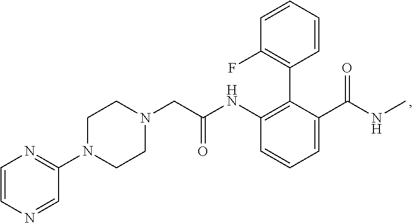 [SMILES: CNC(=O)c1cccc(NC(=O)CN2CCN(c3cnccn3)CC2)c1-c1ccccc1F] | (pw) |
| 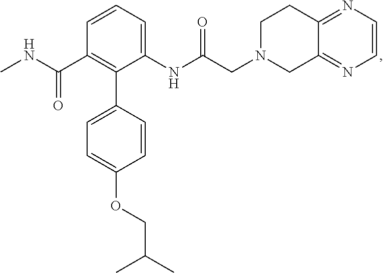 [SMILES: CNC(=O)c1cccc(NC(=O)CN2CCc3nccnc3C2)c1-c1ccc(OCC(C)C)cc1] | (px) |
| 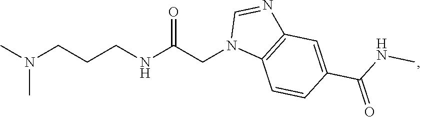 | (py) |
| 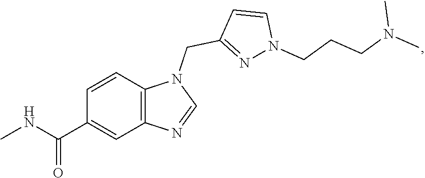 | (pz) |
| 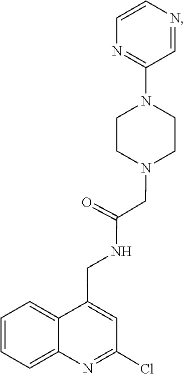 [SMILES: O=C(CN1CCN(c2cnccn2)CC1)NCc1cc(Cl)nc2ccccc12] | (qa) |
| 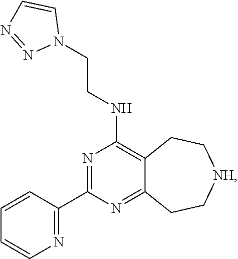 [SMILES: c1ccc(-c2nc3c(c(NCCn4ccnn4)n2)CCNCC3)nc1] | (qb) |
| 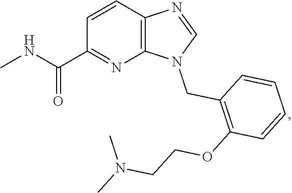 [SMILES: CNC(=O)c1ccc2ncn(Cc3ccccc3OCCN(C)C)c2n1] | (qc) |
| 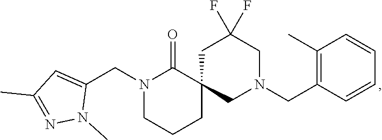 [SMILES: Cc1cc(CN2CCC[C@]3(CN(Cc4ccccc4C)CC(F)(F)C3)C2=O)n(C)n1] | (qd) |
| 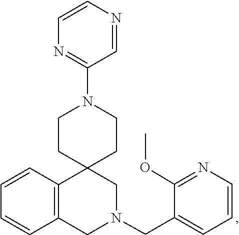 [SMILES: COc1ncccc1CN1Cc2ccccc2C2(CCN(c3cnccn3)CC2)C1] | (qe) |
| 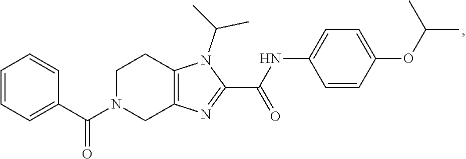 [SMILES: CC(C)Oc1ccc(NC(=O)c2nc3c(n2C(C)C)CCN(C(=O)c2ccccc2)C3)cc1] | (qf) |
| 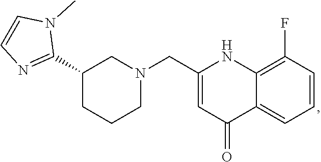 [SMILES: Cn1ccnc1[C@H]1CCCN(Cc2cc(=O)c3cccc(F)c3[nH]2)C1] | (qg) |
| 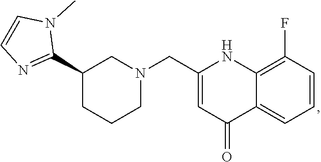 [SMILES: Cn1ccnc1[C@@H]1CCCN(Cc2cc(=O)c3cccc(F)c3[nH]2)C1] | (qh) |
| 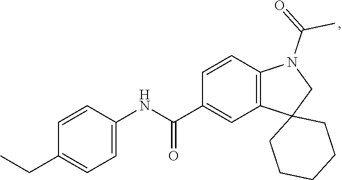 [SMILES: CCc1ccc(NC(=O)c2ccc3c(c2)C2(CCCCC2)CN3C(C)=O)cc1] | (qi) |
| 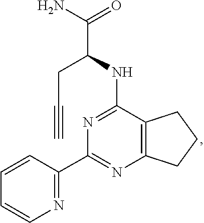 [SMILES: C#CC[C@H](Nc1nc(-c2ccccn2)nc2c1CCC2)C(N)=O] | (qj) |
| 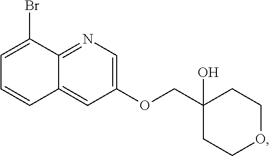 [SMILES: OC1(COc2cnc3c(Br)cccc3c2)CCOCC1] | (qk) |
| 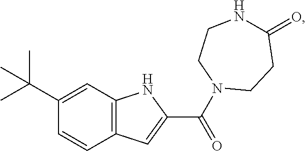 [SMILES: CC(C)(C)c1ccc2cc(C(=O)N3CCNC(=O)CC3)[nH]c2c1] | (ql) |
| 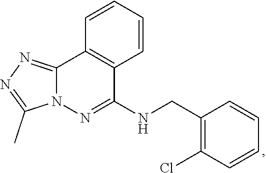 [SMILES: Cc1nnc2c3ccccc3c(NCc3ccccc3Cl)nn12] | (qm) |
| 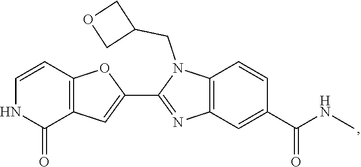 [SMILES: CNC(=O)c1ccc2c(c1)nc(-c1cc3c(=O)[nH]ccc3o1)n2CC1COC1] | (qn) |
| 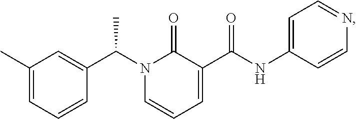 [SMILES: Cc1cccc([C@H](C)n2cccc(C(=O)Nc3ccncc3)c2=O)c1] | (qo) |
| 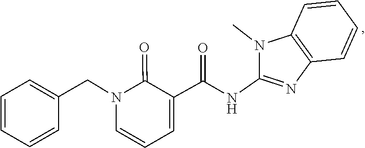 [SMILES: Cn1c(NC(=O)c2cccn(Cc3ccccc3)c2=O)nc2ccccc21] | (qp) |
| 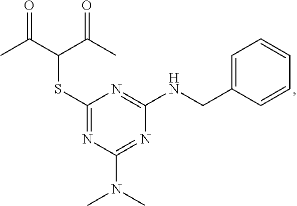 [SMILES: CC(=O)C(Sc1nc(NCc2ccccc2)nc(N(C)C)n1)C(C)=O] | (qq) |
| 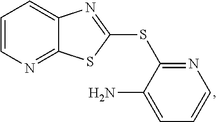 [SMILES: Nc1cccnc1Sc1nc2cccnc2s1] | (qr) |
| 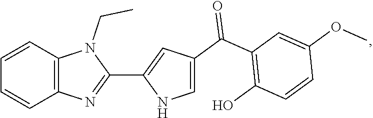 [SMILES: CCn1c(-c2cc(C(=O)c3cc(OC)ccc3O)c[nH]2)nc2ccccc21] | (qs) |
| 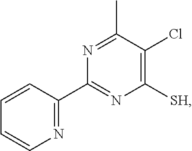 | (qt) |
| 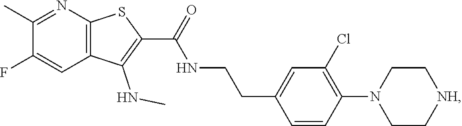 [SMILES: CNc1c(C(=O)NCCc2ccc(N3CCNCC3)c(Cl)c2)sc2nc(C)c(F)cc12] | (qu) |
| 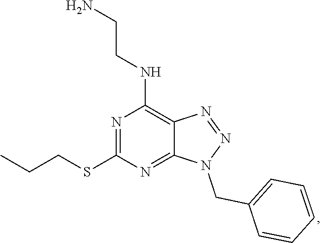 [SMILES: CCCSc1nc(NCCN)c2nnn(Cc3ccccc3)c2n1] | (qv) |
| 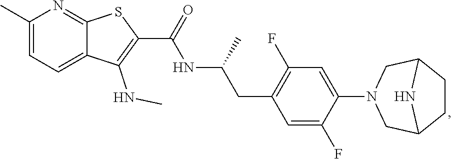 [SMILES: CNc1c(C(=O)N[C@H](C)Cc2cc(F)c(N3CC4CCC(C3)N4)cc2F)sc2nc(C)ccc12] | (qw) |
| 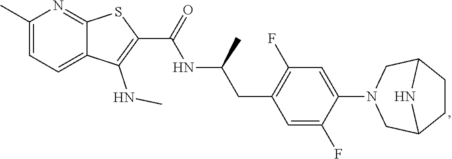 [SMILES: CNc1c(C(=O)N[C@@H](C)Cc2cc(F)c(N3CC4CCC(C3)N4)cc2F)sc2nc(C)ccc12] | (qx) |
| 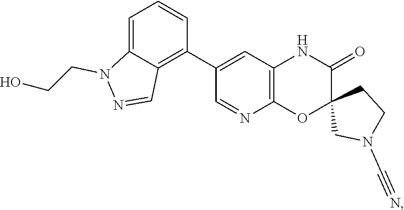 | (qy) |
| 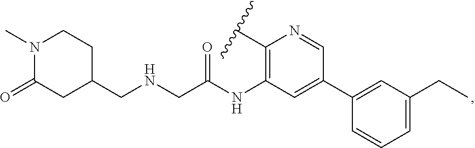 [SMILES: *c1ncc(-c2cccc(CC)c2)cc1NC(=O)CNCC1CCN(C)C(=O)C1] | (qw) |
| 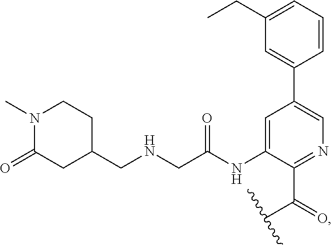 [SMILES: *C(=O)c1ncc(-c2cccc(CC)c2)cc1NC(=O)CNCC1CCN(C)C(=O)C1] | (qx) |
| 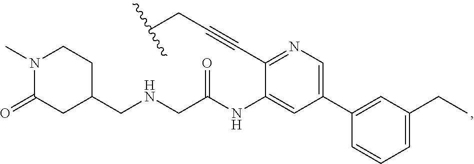 [SMILES: *CC#Cc1ncc(-c2cccc(CC)c2)cc1NC(=O)CNCC1CCN(C)C(=O)C1] | (qy) |
| 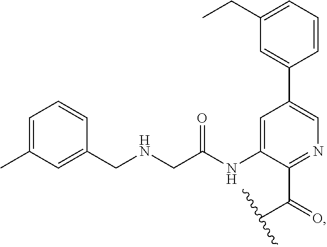 [SMILES: *C(=O)c1ncc(-c2cccc(CC)c2)cc1NC(=O)CNCc1cccc(C)c1] | (qz) |
| 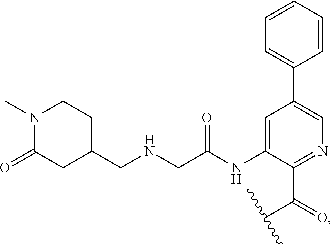 [SMILES: *C(=O)c1ncc(-c2ccccc2)cc1NC(=O)CNCC1CCN(C)C(=O)C1] | (ra) |
| 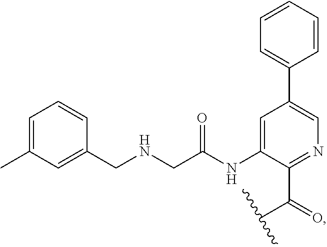 [SMILES: *C(=O)c1ncc(-c2ccccc2)cc1NC(=O)CNCc1cccc(C)c1] | (rb) |
| 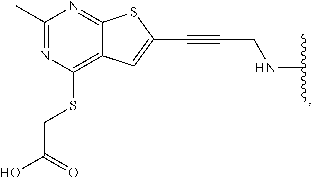 [SMILES: *NCC#Cc1cc2c(SCC(=O)O)nc(C)nc2s1] | (rc) |
| 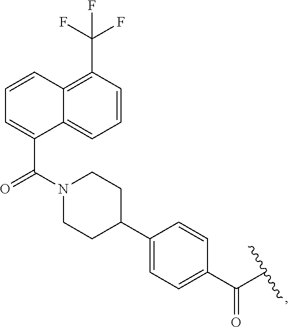 [SMILES: *C(=O)c1ccc(C2CCN(C(=O)c3cccc4c(C(F)(F)F)cccc34)CC2)cc1] | (rd) |
| 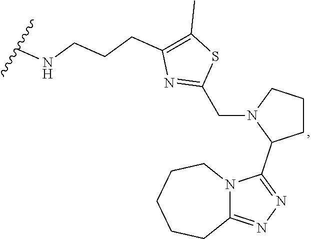 [SMILES: *NCCCc1nc(CN2CCCC2c2nnc3n2CCCCC3)sc1C] | (re) |
| 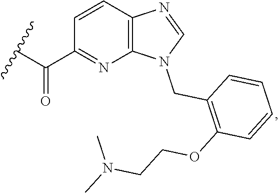 [SMILES: *C(=O)c1ccc2ncn(Cc3ccccc3OCCN(C)C)c2n1] | (rf) |
| 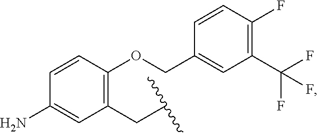 [SMILES: *Cc1cc(N)ccc1OCc1ccc(F)c(C(F)(F)F)c1] | (rg) |
| 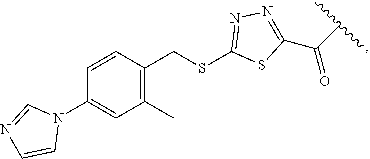 [SMILES: *C(=O)c1nnc(SCc2ccc(-n3ccnc3)cc2C)s1] | (rh) |
| 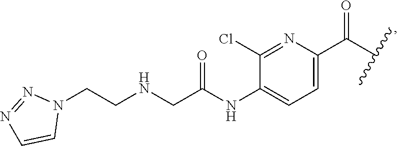 [SMILES: *C(=O)c1ccc(NC(=O)CNCCn2ccnn2)c(Cl)n1] | (ri) |
| 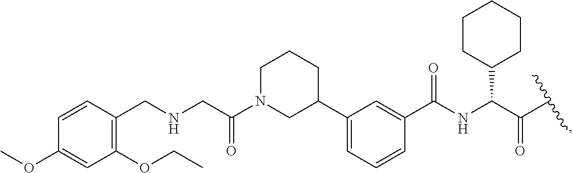 [SMILES: *C(=O)[C@H](NC(=O)c1cccc(C2CCCN(C(=O)CNCc3ccc(OC)cc3OCC)C2)c1)C1CCCCC1] | (rj) |
| 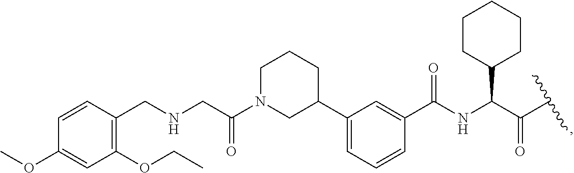 [SMILES: *C(=O)[C@@H](NC(=O)c1cccc(C2CCCN(C(=O)CNCc3ccc(OC)cc3OCC)C2)c1)C1CCCCC1] | (rk) |
| 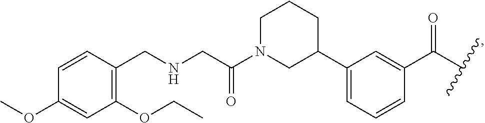 [SMILES: *C(=O)c1cccc(C2CCCN(C(=O)CNCc3ccc(OC)cc3OCC)C2)c1] | (rl) |
| 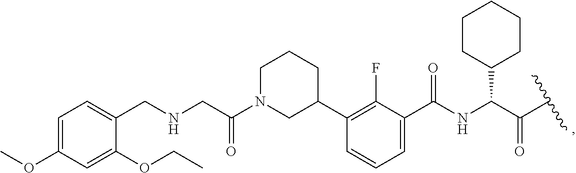 [SMILES: *C(=O)[C@H](NC(=O)c1cccc(C2CCCN(C(=O)CNCc3ccc(OC)cc3OCC)C2)c1F)C1CCCCC1] | (rm) |
| 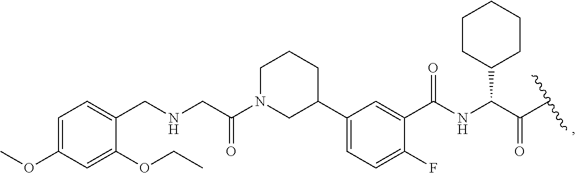 [SMILES: *C(=O)[C@H](NC(=O)c1cc(C2CCCN(C(=O)CNCc3ccc(OC)cc3OCC)C2)ccc1F)C1CCCCC1] | (rn) |
| 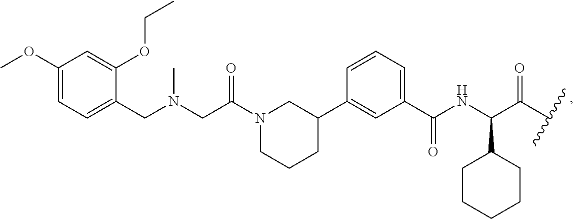 [SMILES: *C(=O)[C@H](NC(=O)c1cccc(C2CCCN(C(=O)CN(C)Cc3ccc(OC)cc3OCC)C2)c1)C1CCCCC1] | (ro) |
| 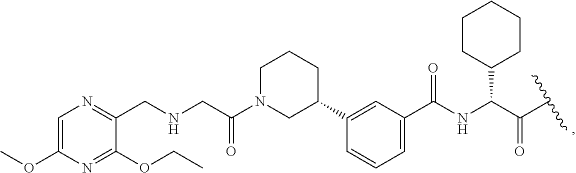 [SMILES: *C(=O)[C@H](NC(=O)c1cccc([C@H]2CCCN(C(=O)CNCc3ncc(OC)nc3OCC)C2)c1)C1CCCCC1] | (rp) |
| 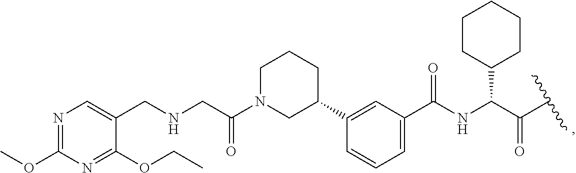 [SMILES: *C(=O)[C@H](NC(=O)c1cccc([C@H]2CCCN(C(=O)CNCc3cnc(OC)nc3OCC)C2)c1)C1CCCCC1] | (rq) |
| 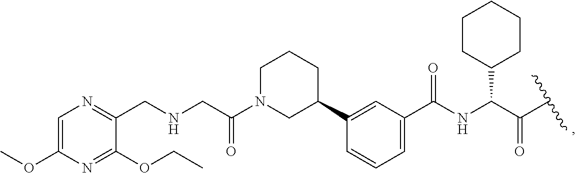 [SMILES: *C(=O)[C@H](NC(=O)c1cccc([C@@H]2CCCN(C(=O)CNCc3ncc(OC)nc3OCC)C2)c1)C1CCCCC1] | (rr) |
| 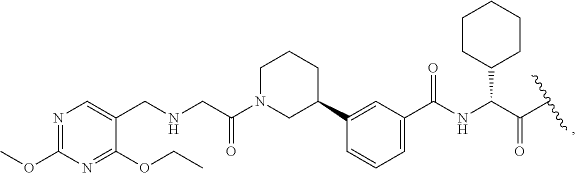 [SMILES: *C(=O)[C@H](NC(=O)c1cccc([C@@H]2CCCN(C(=O)CNCc3cnc(OC)nc3OCC)C2)c1)C1CCCCC1] | (rs) |
| 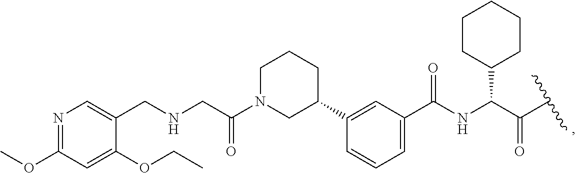 [SMILES: *C(=O)[C@H](NC(=O)c1cccc([C@H]2CCCN(C(=O)CNCc3cnc(OC)cc3OCC)C2)c1)C1CCCCC1] | (rt) |
| 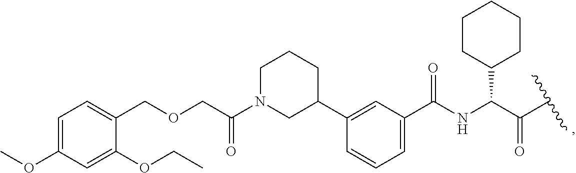 [SMILES: *C(=O)[C@H](NC(=O)c1cccc(C2CCCN(C(=O)COCc3ccc(OC)cc3OCC)C2)c1)C1CCCCC1] | (ru) |
| 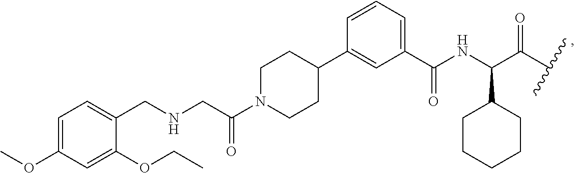 [SMILES: *C(=O)[C@H](NC(=O)c1cccc(C2CCN(C(=O)CNCc3ccc(OC)cc3OCC)CC2)c1)C1CCCCC1] | (rv) |
| 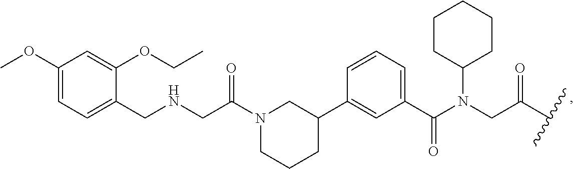 [SMILES: *C(=O)CN(C(=O)c1cccc(C2CCCN(C(=O)CNCc3ccc(OC)cc3OCC)C2)c1)C1CCCCC1] | (rw) |
| 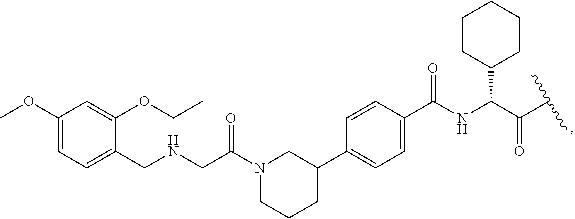 [SMILES: *C(=O)[C@H](NC(=O)c1ccc(C2CCCN(C(=O)CNCc3ccc(OC)cc3OCC)C2)cc1)C1CCCCC1] | (rx) |
| 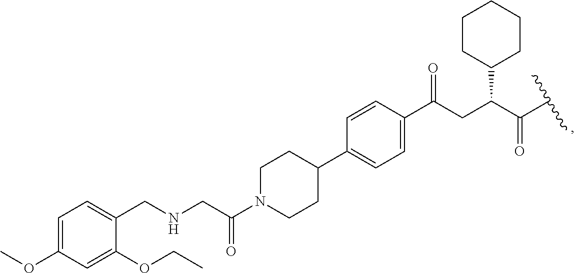 [SMILES: *C(=O)[C@H](CC(=O)c1ccc(C2CCN(C(=O)CNCc3ccc(OC)cc3OCC)CC2)cc1)C1CCCCC1] | (ry) |
| 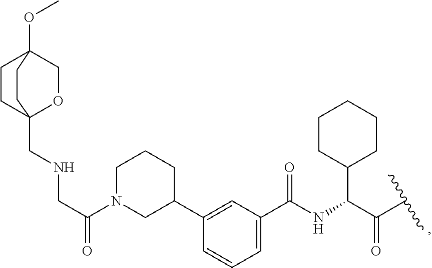 [SMILES: *C(=O)[C@H](NC(=O)c1cccc(C2CCCN(C(=O)CNCC34CCC(OC)(CC3)CO4)C2)c1)C1CCCCC1] | (rz) |
| 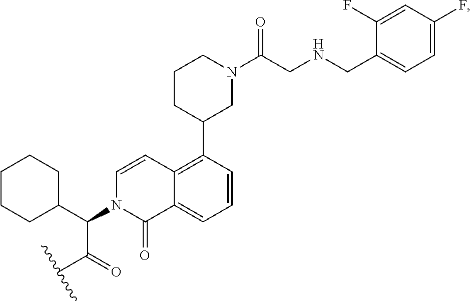 [SMILES: *C(=O)[C@@H](C1CCCCC1)n1ccc2c(C3CCCN(C(=O)CNCc4ccc(F)cc4F)C3)cccc2c1=O] | (sa) |
| 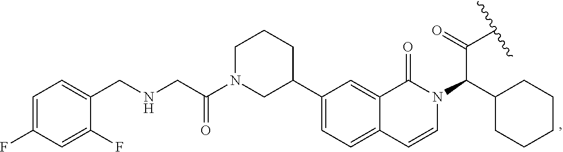 [SMILES: *C(=O)[C@@H](C1CCCCC1)n1ccc2ccc(C3CCCN(C(=O)CNCc4ccc(F)cc4F)C3)cc2c1=O] | (sb) |
| 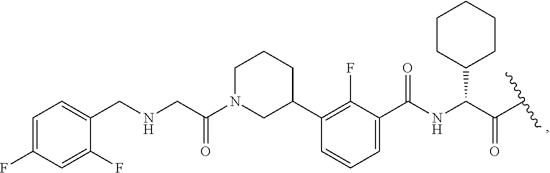 [SMILES: *C(=O)[C@H](NC(=O)c1cccc(C2CCCN(C(=O)CNCc3ccc(F)cc3F)C2)c1F)C1CCCCC1] | (sc) |
| 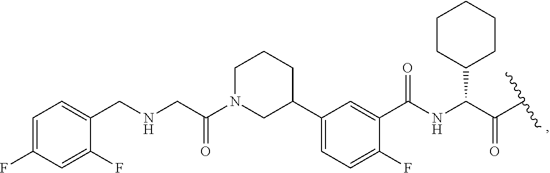 [SMILES: *C(=O)[C@H](NC(=O)c1cc(C2CCCN(C(=O)CNCc3ccc(F)cc3F)C2)ccc1F)C1CCCCC1] | (sd) |
| 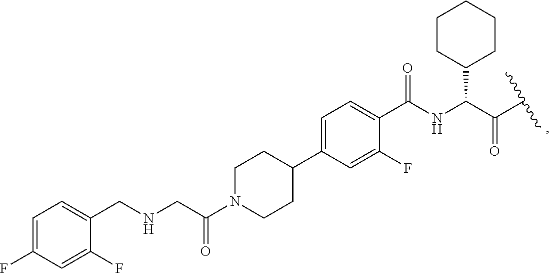 [SMILES: *C(=O)[C@H](NC(=O)c1ccc(C2CCN(C(=O)CNCc3ccc(F)cc3F)CC2)cc1F)C1CCCCC1] | (se) |
| 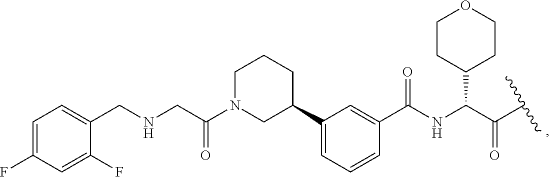 [SMILES: *C(=O)[C@H](NC(=O)c1cccc([C@@H]2CCCN(C(=O)CNCc3ccc(F)cc3F)C2)c1)C1CCOCC1] | (sf) |
| 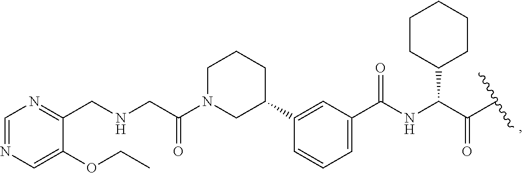 [SMILES: *C(=O)[C@H](NC(=O)c1cccc([C@H]2CCCN(C(=O)CNCc3ncncc3OCC)C2)c1)C1CCCCC1] | (sg) |
| 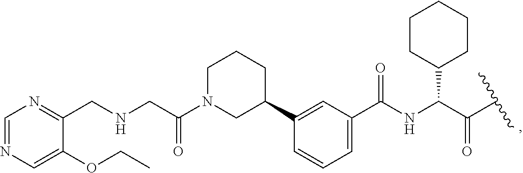 [SMILES: *C(=O)[C@H](NC(=O)c1cccc([C@@H]2CCCN(C(=O)CNCc3ncncc3OCC)C2)c1)C1CCCCC1] | (sh) |
| 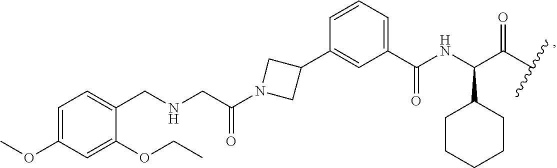 [SMILES: *C(=O)[C@H](NC(=O)c1cccc(C2CN(C(=O)CNCc3ccc(OC)cc3OCC)C2)c1)C1CCCCC1] | (si) |
| 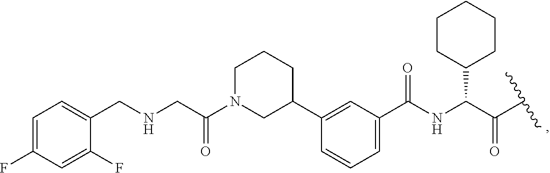 [SMILES: *C(=O)[C@H](NC(=O)c1cccc(C2CCCN(C(=O)CNCc3ccc(F)cc3F)C2)c1)C1CCCCC1] | (sj) |
| 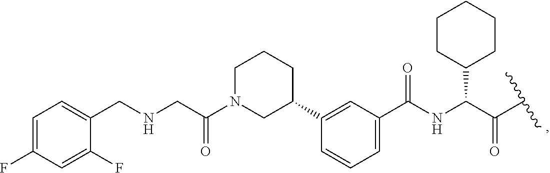 [SMILES: *C(=O)[C@H](NC(=O)c1cccc([C@H]2CCCN(C(=O)CNCc3ccc(F)cc3F)C2)c1)C1CCCCC1] | (sk) |
| 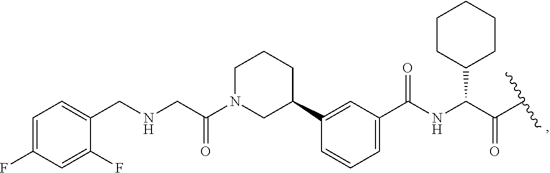 [SMILES: *C(=O)[C@H](NC(=O)c1cccc([C@@H]2CCCN(C(=O)CNCc3ccc(F)cc3F)C2)c1)C1CCCCC1] | (sl) |
| 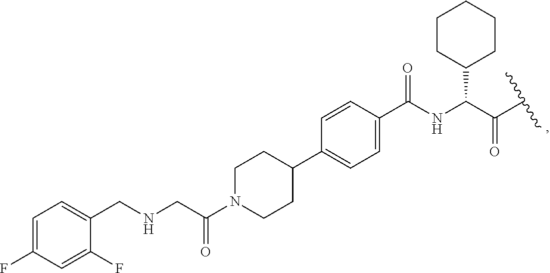 [SMILES: *C(=O)[C@H](NC(=O)c1ccc(C2CCN(C(=O)CNCc3ccc(F)cc3F)CC2)cc1)C1CCCCC1] | (sm) |
| 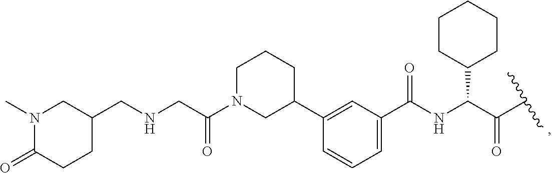 [SMILES: *C(=O)[C@H](NC(=O)c1cccc(C2CCCN(C(=O)CNCC3CCC(=O)N(C)C3)C2)c1)C1CCCCC1] | (sn) |
| 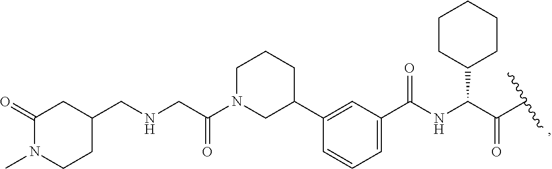 [SMILES: *C(=O)[C@H](NC(=O)c1cccc(C2CCCN(C(=O)CNCC3CCN(C)C(=O)C3)C2)c1)C1CCCCC1] | (so) |
| 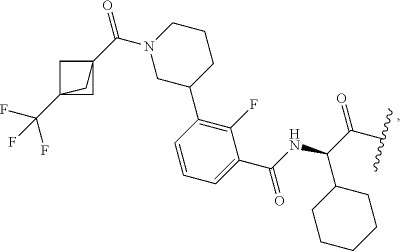 [SMILES: *C(=O)[C@H](NC(=O)c1cccc(C2CCCN(C(=O)C34CC(C(F)(F)F)(C3)C4)C2)c1F)C1CCCCC1] | (sp) |
| 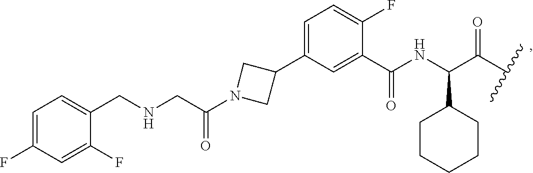 [SMILES: *C(=O)[C@H](NC(=O)c1cc(C2CN(C(=O)CNCc3ccc(F)cc3F)C2)ccc1F)C1CCCCC1] | (sq) |
| 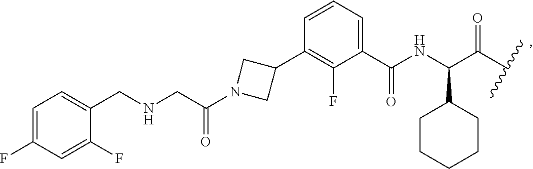 [SMILES: *C(=O)[C@H](NC(=O)c1cccc(C2CN(C(=O)CNCc3ccc(F)cc3F)C2)c1F)C1CCCCC1] | (sr) |
| 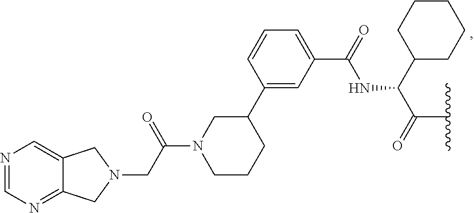 [SMILES: *C(=O)[C@H](NC(=O)c1cccc(C2CCCN(C(=O)CN3Cc4cncnc4C3)C2)c1)C1CCCCC1] | (ss) |
| 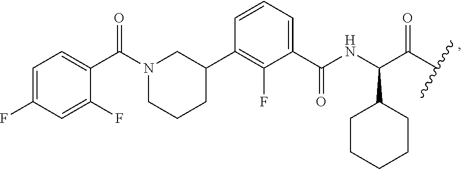 [SMILES: *C(=O)[C@H](NC(=O)c1cccc(C2CCCN(C(=O)c3ccc(F)cc3F)C2)c1F)C1CCCCC1] | (st) |
| 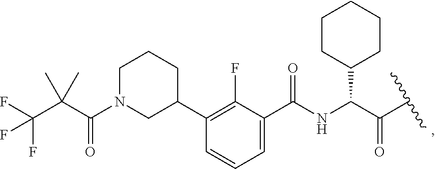 [SMILES: *C(=O)[C@H](NC(=O)c1cccc(C2CCCN(C(=O)C(C)(C)C(F)(F)F)C2)c1F)C1CCCCC1] | (su) |
| 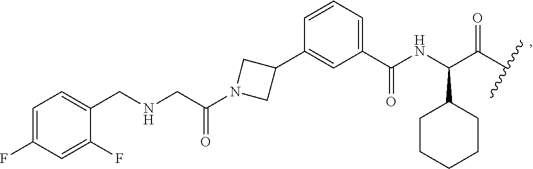 [SMILES: *C(=O)[C@H](NC(=O)c1cccc(C2CN(C(=O)CNCc3ccc(F)cc3F)C2)c1)C1CCCCC1] | (sv) |
| 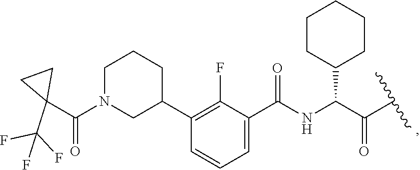 [SMILES: *C(=O)[C@H](NC(=O)c1cccc(C2CCCN(C(=O)C3(C(F)(F)F)CC3)C2)c1F)C1CCCCC1] | (sw) |
| 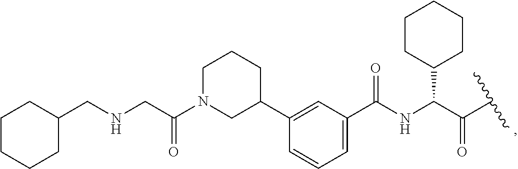 [SMILES: *C(=O)[C@H](NC(=O)c1cccc(C2CCCN(C(=O)CNCC3CCCCC3)C2)c1)C1CCCCC1] | (sx) |
| 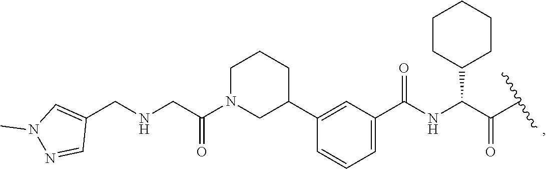 [SMILES: *C(=O)[C@H](NC(=O)c1cccc(C2CCCN(C(=O)CNCc3cnn(C)c3)C2)c1)C1CCCCC1] | (sy) |
| 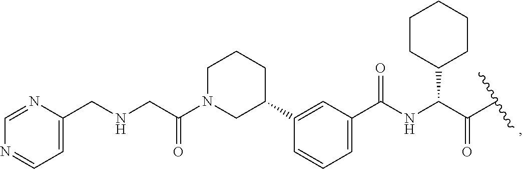 [SMILES: *C(=O)[C@H](NC(=O)c1cccc([C@H]2CCCN(C(=O)CNCc3ccncn3)C2)c1)C1CCCCC1] | (sz) |
| 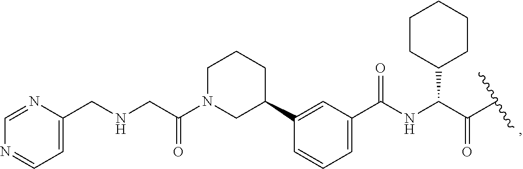 [SMILES: *C(=O)[C@H](NC(=O)c1cccc([C@@H]2CCCN(C(=O)CNCc3ccncn3)C2)c1)C1CCCCC1] | (ta) |
| 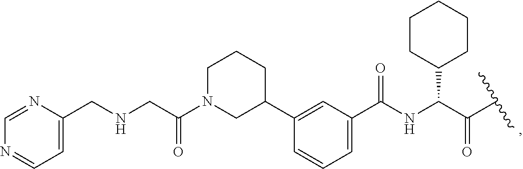 [SMILES: *C(=O)[C@H](NC(=O)c1cccc(C2CCCN(C(=O)CNCc3ccncn3)C2)c1)C1CCCCC1] | (tb) |
| 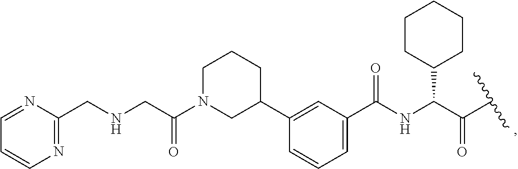 [SMILES: *C(=O)[C@H](NC(=O)c1cccc(C2CCCN(C(=O)CNCc3ncccn3)C2)c1)C1CCCCC1] | (tc) |
| 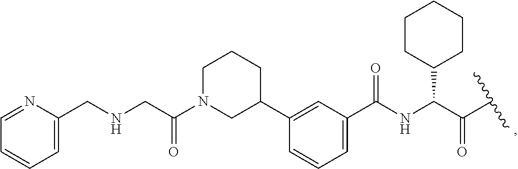 [SMILES: *C(=O)[C@H](NC(=O)c1cccc(C2CCCN(C(=O)CNCc3ccccn3)C2)c1)C1CCCCC1] | (td) |
| 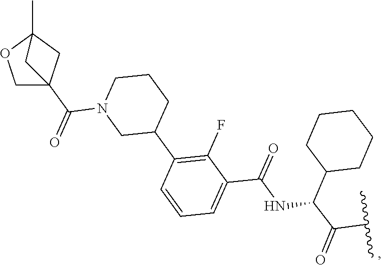 [SMILES: *C(=O)[C@H](NC(=O)c1cccc(C2CCCN(C(=O)C34COC(C)(C3)C4)C2)c1F)C1CCCCC1] | (te) |
| 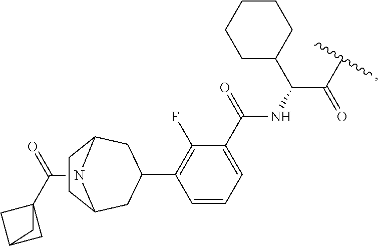 [SMILES: *C(=O)[C@H](NC(=O)c1cccc(C2CC3CCC(C2)N3C(=O)C23CC(C2)C3)c1F)C1CCCCC1] | (tf) |
| 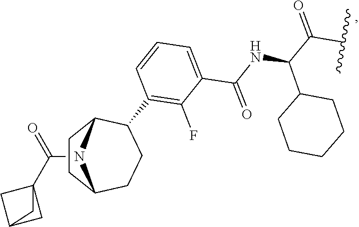 [SMILES: *C(=O)[C@H](NC(=O)c1cccc([C@@H]2CC[C@@H]3CC[C@H]2N3C(=O)C23CC(C2)C3)c1F)C1CCCCC1] | (tg) |
| 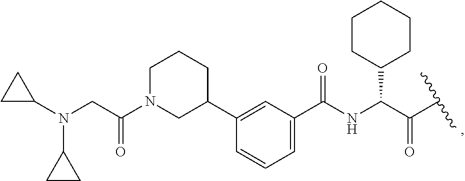 [SMILES: *C(=O)[C@H](NC(=O)c1cccc(C2CCCN(C(=O)CN(C3CC3)C3CC3)C2)c1)C1CCCCC1] | (th) |
| 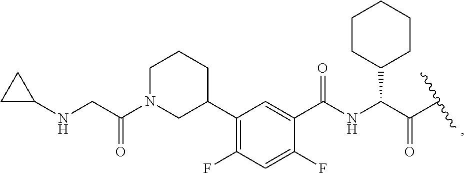 [SMILES: *C(=O)[C@H](NC(=O)c1cc(C2CCCN(C(=O)CNC3CC3)C2)c(F)cc1F)C1CCCCC1] | (ti) |
| 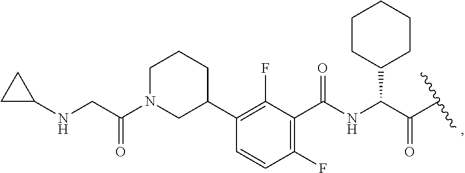 [SMILES: *C(=O)[C@H](NC(=O)c1c(F)ccc(C2CCCN(C(=O)CNC3CC3)C2)c1F)C1CCCCC1] | (tj) |
| 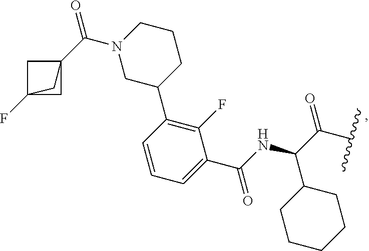 [SMILES: *C(=O)[C@H](NC(=O)c1cccc(C2CCCN(C(=O)C34CC(F)(C3)C4)C2)c1F)C1CCCCC1] | (tk) |
| 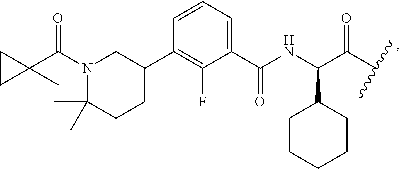 [SMILES: *C(=O)[C@H](NC(=O)c1cccc(C2CCC(C)(C)N(C(=O)C3(C)CC3)C2)c1F)C1CCCCC1] | (tl) |
| 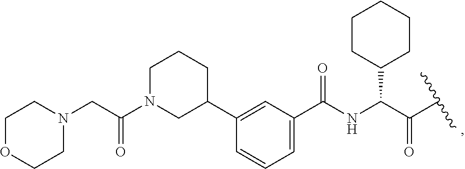 [SMILES: *C(=O)[C@H](NC(=O)c1cccc(C2CCCN(C(=O)CN3CCOCC3)C2)c1)C1CCCCC1] | (tm) |
| 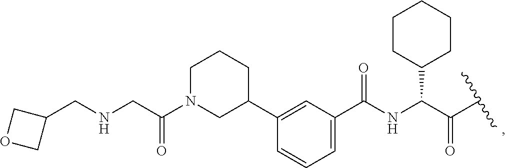 [SMILES: *C(=O)[C@H](NC(=O)c1cccc(C2CCCN(C(=O)CNCC3COC3)C2)c1)C1CCCCC1] | (tn) |
| 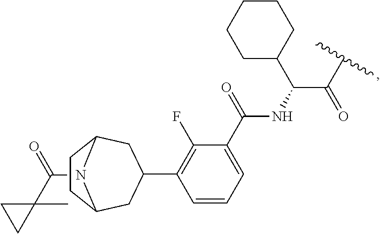 [SMILES: *C(=O)[C@H](NC(=O)c1cccc(C2CC3CCC(C2)N3C(=O)C2(C)CC2)c1F)C1CCCCC1] | (to) |
| 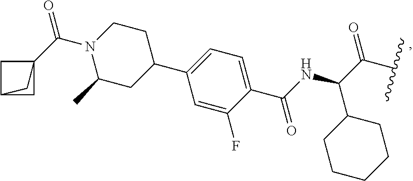 [SMILES: *C(=O)[C@H](NC(=O)c1ccc(C2CCN(C(=O)C34CC(C3)C4)[C@H](C)C2)cc1F)C1CCCCC1] | (tp) |
| 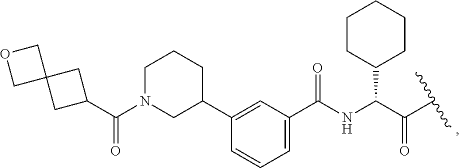 [SMILES: *C(=O)[C@H](NC(=O)c1cccc(C2CCCN(C(=O)C3CC4(COC4)C3)C2)c1)C1CCCCC1] | (tq) |
| 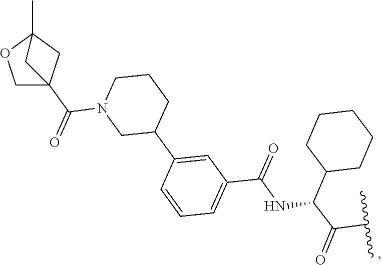 [SMILES: *C(=O)[C@H](NC(=O)c1cccc(C2CCCN(C(=O)C34COC(C)(C3)C4)C2)c1)C1CCCCC1] | (tr) |
| 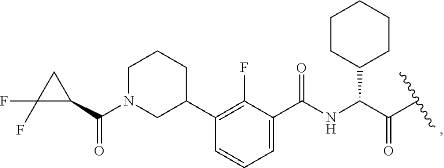 [SMILES: *C(=O)[C@H](NC(=O)c1cccc(C2CCCN(C(=O)[C@@H]3CC3(F)F)C2)c1F)C1CCCCC1] | (ts) |
| 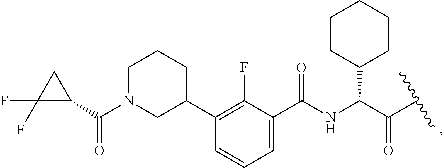 [SMILES: *C(=O)[C@H](NC(=O)c1cccc(C2CCCN(C(=O)[C@H]3CC3(F)F)C2)c1F)C1CCCCC1] | (tt) |
| 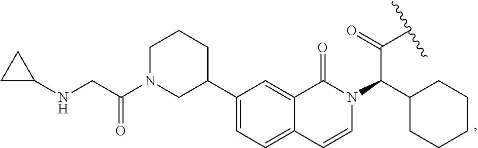 [SMILES: *C(=O)[C@@H](C1CCCCC1)n1ccc2ccc(C3CCCN(C(=O)CNC4CC4)C3)cc2c1=O] | (tu) |
| 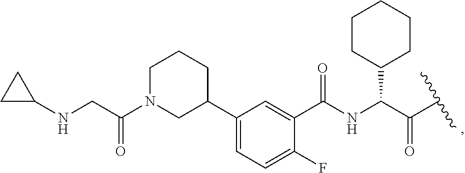 [SMILES: *C(=O)[C@H](NC(=O)c1cc(C2CCCN(C(=O)CNC3CC3)C2)ccc1F)C1CCCCC1] | (tv) |
| 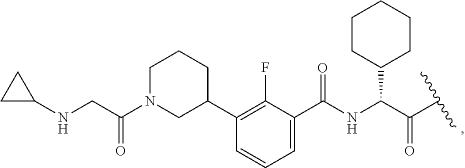 [SMILES: *C(=O)[C@H](NC(=O)c1cccc(C2CCCN(C(=O)CNC3CC3)C2)c1F)C1CCCCC1] | (tw) |
| 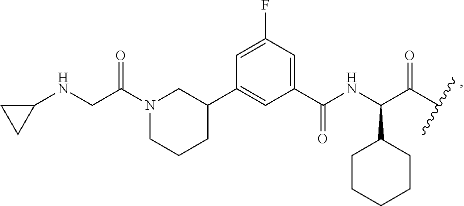 [SMILES: *C(=O)[C@H](NC(=O)c1cc(F)cc(C2CCCN(C(=O)CNC3CC3)C2)c1)C1CCCCC1] | (tx) |
| 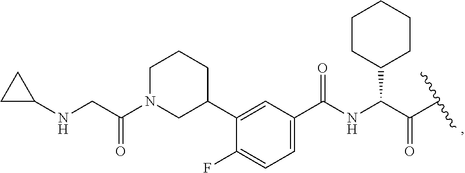 [SMILES: *C(=O)[C@H](NC(=O)c1ccc(F)c(C2CCCN(C(=O)CNC3CC3)C2)c1)C1CCCCC1] | (ty) |
| 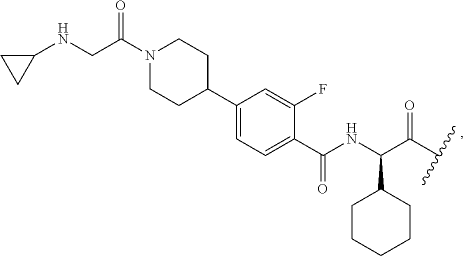 [SMILES: *C(=O)[C@H](NC(=O)c1ccc(C2CCN(C(=O)CNC3CC3)CC2)cc1F)C1CCCCC1] | (tz) |
| 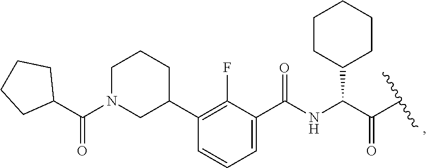 [SMILES: *C(=O)[C@H](NC(=O)c1cccc(C2CCCN(C(=O)C3CCCC3)C2)c1F)C1CCCCC1] | (ua) |
| 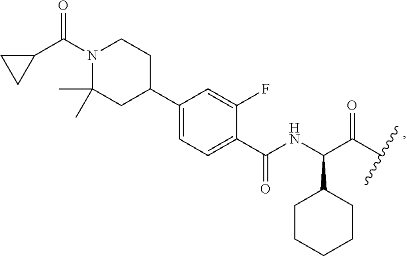 [SMILES: *C(=O)[C@H](NC(=O)c1ccc(C2CCN(C(=O)C3CC3)C(C)(C)C2)cc1F)C1CCCCC1] | (ub) |
| 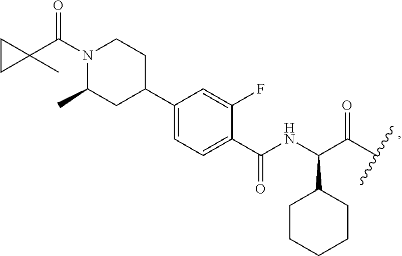 [SMILES: *C(=O)[C@H](NC(=O)c1ccc(C2CCN(C(=O)C3(C)CC3)[C@H](C)C2)cc1F)C1CCCCC1] | (uc) |
| 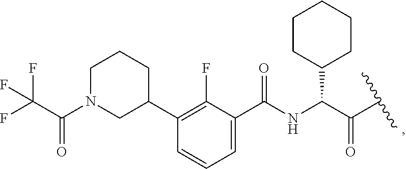 [SMILES: *C(=O)[C@H](NC(=O)c1cccc(C2CCCN(C(=O)C(F)(F)F)C2)c1F)C1CCCCC1] | (ud) |
| 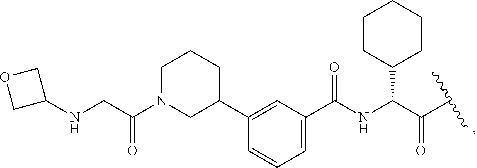 [SMILES: *C(=O)[C@H](NC(=O)c1cccc(C2CCCN(C(=O)CNC3COC3)C2)c1)C1CCCCC1] | (ue) |
| 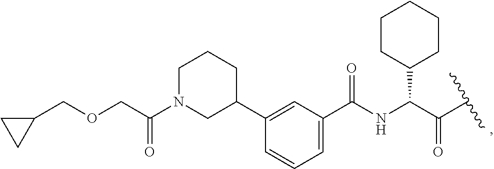 [SMILES: *C(=O)[C@H](NC(=O)c1cccc(C2CCCN(C(=O)COCC3CC3)C2)c1)C1CCCCC1] | (uf) |
| 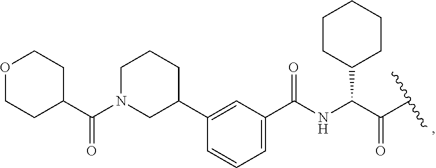 [SMILES: *C(=O)[C@H](NC(=O)c1cccc(C2CCCN(C(=O)C3CCOCC3)C2)c1)C1CCCCC1] | (ug) |
| 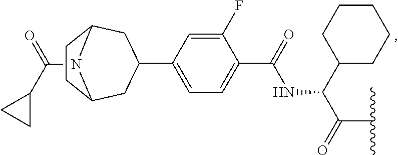 [SMILES: *C(=O)[C@H](NC(=O)c1ccc(C2CC3CCC(C2)N3C(=O)C2CC2)cc1F)C1CCCCC1] | (uh) |
| 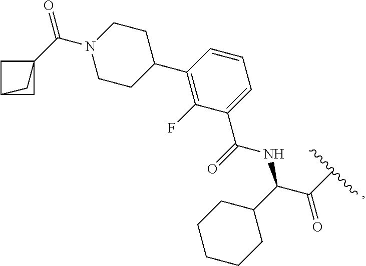 [SMILES: *C(=O)[C@H](NC(=O)c1cccc(C2CCN(C(=O)C34CC(C3)C4)CC2)c1F)C1CCCCC1] | (ui) |
| 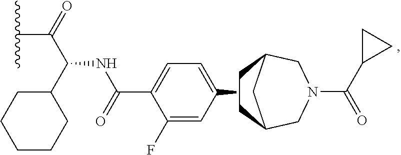 | (uj) |
| 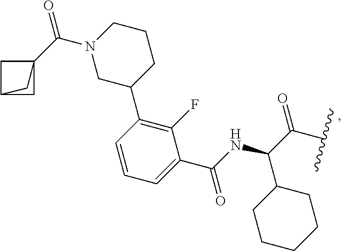 [SMILES: *C(=O)[C@H](NC(=O)c1cccc(C2CCCN(C(=O)C34CC(C3)C4)C2)c1F)C1CCCCC1] | (uk) |
| 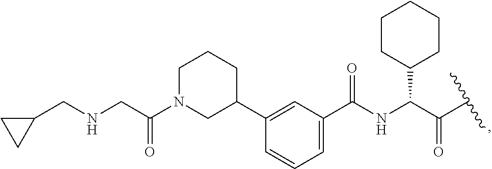 [SMILES: *C(=O)[C@H](NC(=O)c1cccc(C2CCCN(C(=O)CNCC3CC3)C2)c1)C1CCCCC1] | (ul) |
| 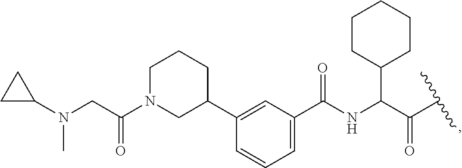 [SMILES: *C(=O)C(NC(=O)c1cccc(C2CCCN(C(=O)CN(C)C3CC3)C2)c1)C1CCCCC1] | (um) |
| 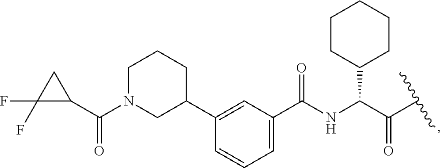 [SMILES: *C(=O)[C@H](NC(=O)c1cccc(C2CCCN(C(=O)C3CC3(F)F)C2)c1)C1CCCCC1] | (un) |
| 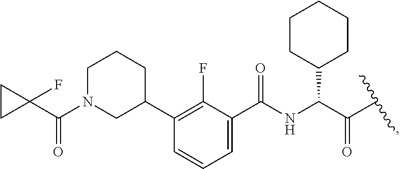 [SMILES: *C(=O)[C@H](NC(=O)c1cccc(C2CCCN(C(=O)C3(F)CC3)C2)c1F)C1CCCCC1] | (uo) |
| 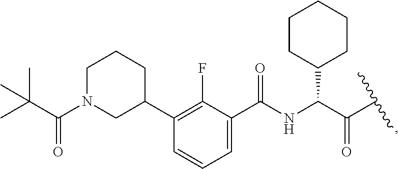 [SMILES: *C(=O)[C@H](NC(=O)c1cccc(C2CCCN(C(=O)C(C)(C)C)C2)c1F)C1CCCCC1] | (up) |
| 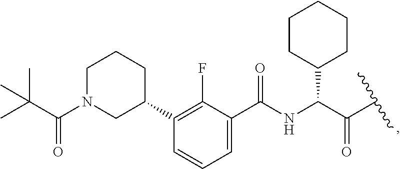 [SMILES: *C(=O)[C@H](NC(=O)c1cccc([C@H]2CCCN(C(=O)C(C)(C)C)C2)c1F)C1CCCCC1] | (uq) |
| 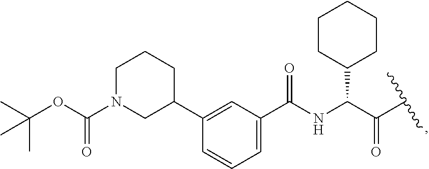 [SMILES: *C(=O)[C@H](NC(=O)c1cccc(C2CCCN(C(=O)OC(C)(C)C)C2)c1)C1CCCCC1] | (ur) |
| 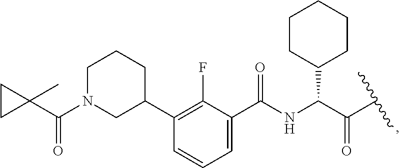 [SMILES: *C(=O)[C@H](NC(=O)c1cccc(C2CCCN(C(=O)C3(C)CC3)C2)c1F)C1CCCCC1] | (us) |
| 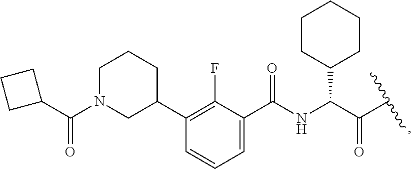 [SMILES: *C(=O)[C@H](NC(=O)c1cccc(C2CCCN(C(=O)C3CCC3)C2)c1F)C1CCCCC1] | (ut) |
| 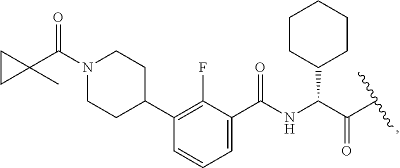 [SMILES: *C(=O)[C@H](NC(=O)c1cccc(C2CCN(C(=O)C3(C)CC3)CC2)c1F)C1CCCCC1] | (uu) |
| 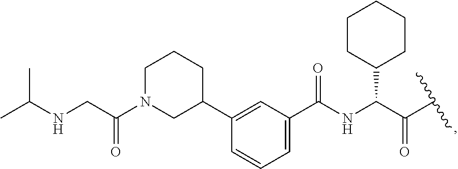 [SMILES: *C(=O)[C@H](NC(=O)c1cccc(C2CCCN(C(=O)CNC(C)C)C2)c1)C1CCCCC1] | (uv) |
| 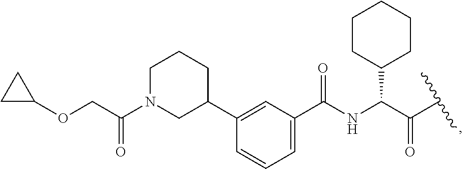 [SMILES: *C(=O)[C@H](NC(=O)c1cccc(C2CCCN(C(=O)COC3CC3)C2)c1)C1CCCCC1] | (uw) |
| 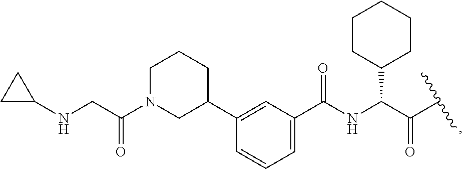 [SMILES: *C(=O)[C@H](NC(=O)c1cccc(C2CCCN(C(=O)CNC3CC3)C2)c1)C1CCCCC1] | (ux) |
| 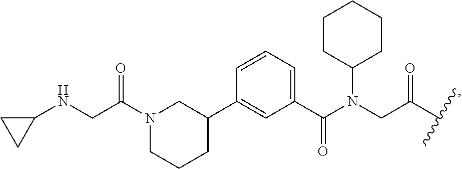 [SMILES: *C(=O)CN(C(=O)c1cccc(C2CCCN(C(=O)CNC3CC3)C2)c1)C1CCCCC1] | (uy) |
| 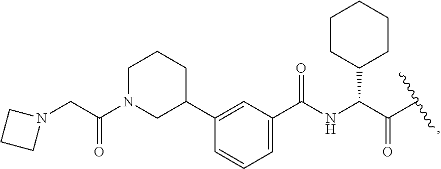 [SMILES: *C(=O)[C@H](NC(=O)c1cccc(C2CCCN(C(=O)CN3CCC3)C2)c1)C1CCCCC1] | (uz) |
| 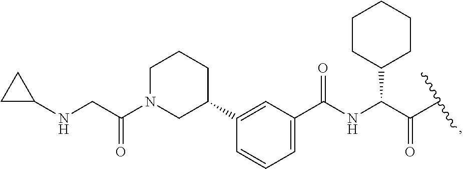 [SMILES: *C(=O)[C@H](NC(=O)c1cccc([C@H]2CCCN(C(=O)CNC3CC3)C2)c1)C1CCCCC1] | (va) |
| 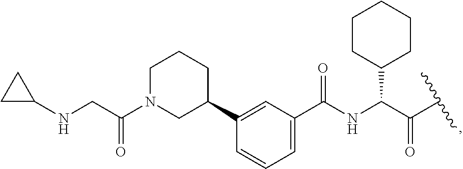 [SMILES: *C(=O)[C@H](NC(=O)c1cccc([C@@H]2CCCN(C(=O)CNC3CC3)C2)c1)C1CCCCC1] | (vb) |
| 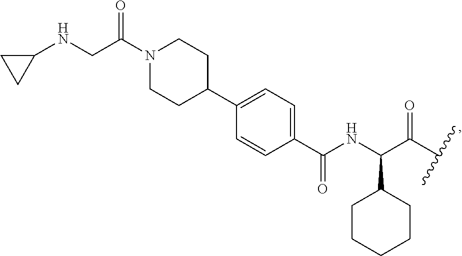 [SMILES: *C(=O)[C@H](NC(=O)c1ccc(C2CCN(C(=O)CNC3CC3)CC2)cc1)C1CCCCC1] | (vc) |
| 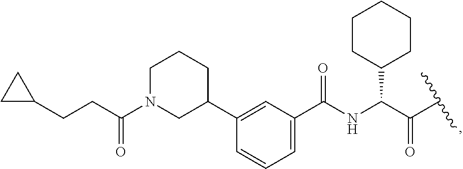 [SMILES: *C(=O)[C@H](NC(=O)c1cccc(C2CCCN(C(=O)CCC3CC3)C2)c1)C1CCCCC1] | (vd) |
| 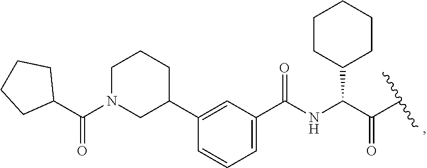 [SMILES: *C(=O)[C@H](NC(=O)c1cccc(C2CCCN(C(=O)C3CCCC3)C2)c1)C1CCCCC1] | (ve) |
| 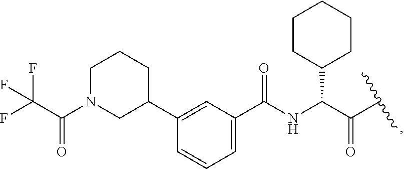 [SMILES: *C(=O)[C@H](NC(=O)c1cccc(C2CCCN(C(=O)C(F)(F)F)C2)c1)C1CCCCC1] | (vf) |
| 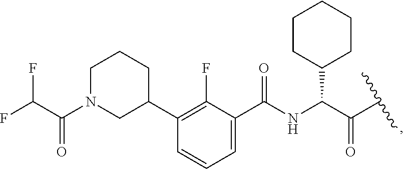 [SMILES: *C(=O)[C@H](NC(=O)c1cccc(C2CCCN(C(=O)C(F)F)C2)c1F)C1CCCCC1] | (vg) |
| 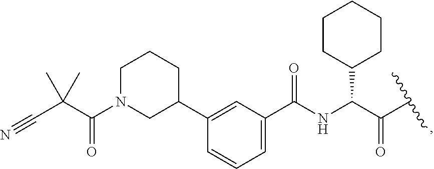 [SMILES: *C(=O)[C@H](NC(=O)c1cccc(C2CCCN(C(=O)C(C)(C)C#N)C2)c1)C1CCCCC1] | (vh) |
| 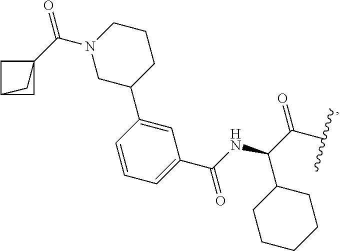 [SMILES: *C(=O)[C@H](NC(=O)c1cccc(C2CCCN(C(=O)C34CC(C3)C4)C2)c1)C1CCCCC1] | (vi) |
| 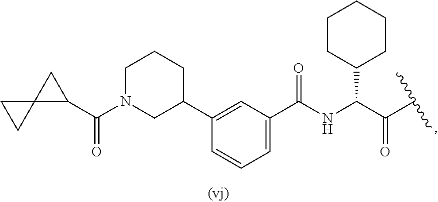 [SMILES: *C(=O)[C@H](NC(=O)c1cccc(C2CCCN(C(=O)C3CC34CC4)C2)c1)C1CCCCC1] | (vj) |
| 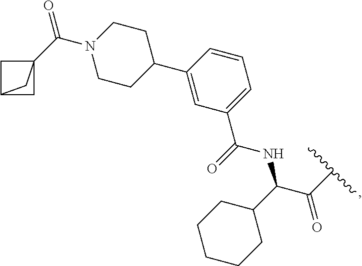 [SMILES: *C(=O)[C@H](NC(=O)c1cccc(C2CCN(C(=O)C34CC(C3)C4)CC2)c1)C1CCCCC1] | (vk) |
| 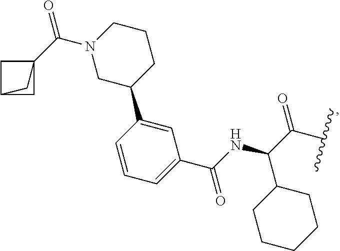 [SMILES: *C(=O)[C@H](NC(=O)c1cccc([C@@H]2CCCN(C(=O)C34CC(C3)C4)C2)c1)C1CCCCC1] | (vl) |
| 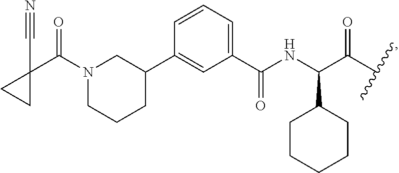 [SMILES: *C(=O)[C@H](NC(=O)c1cccc(C2CCCN(C(=O)C3(C#N)CC3)C2)c1)C1CCCCC1] | (vm) |
| 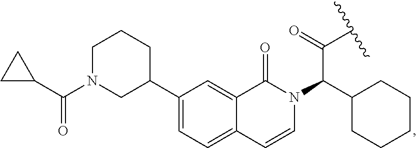 [SMILES: *C(=O)[C@@H](C1CCCCC1)n1ccc2ccc(C3CCCN(C(=O)C4CC4)C3)cc2c1=O] | (vn) |
| 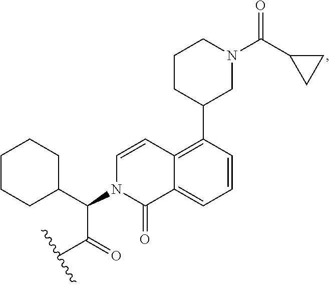 [SMILES: *C(=O)[C@@H](C1CCCCC1)n1ccc2c(C3CCCN(C(=O)C4CC4)C3)cccc2c1=O] | (vo) |
| 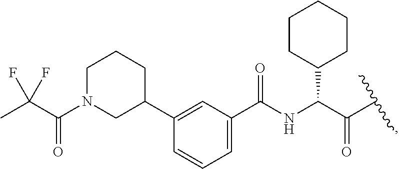 [SMILES: *C(=O)[C@H](NC(=O)c1cccc(C2CCCN(C(=O)C(C)(F)F)C2)c1)C1CCCCC1] | (vp) |
| 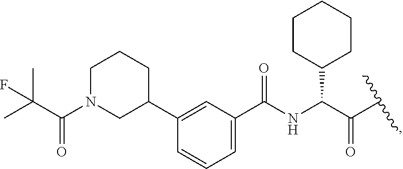 [SMILES: *C(=O)[C@H](NC(=O)c1cccc(C2CCCN(C(=O)C(C)(C)F)C2)c1)C1CCCCC1] | (vq) |
| 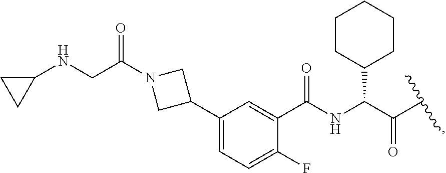 [SMILES: *C(=O)[C@H](NC(=O)c1cc(C2CN(C(=O)CNC3CC3)C2)ccc1F)C1CCCCC1] | (vr) |
| 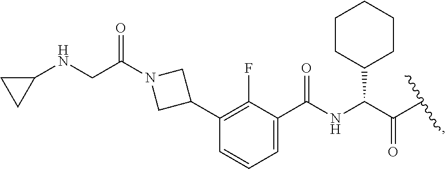 [SMILES: *C(=O)[C@H](NC(=O)c1cccc(C2CN(C(=O)CNC3CC3)C2)c1F)C1CCCCC1] | (vs) |
| 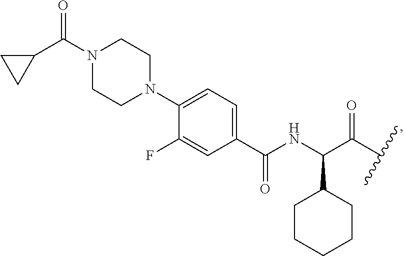 [SMILES: *C(=O)[C@H](NC(=O)c1ccc(N2CCN(C(=O)C3CC3)CC2)c(F)c1)C1CCCCC1] | (vt) |
| 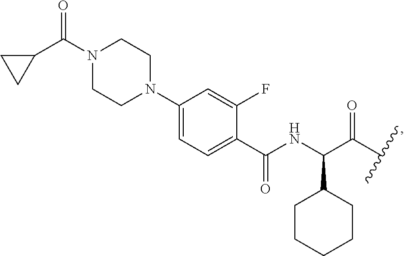 [SMILES: *C(=O)[C@H](NC(=O)c1ccc(N2CCN(C(=O)C3CC3)CC2)cc1F)C1CCCCC1] | (vu) |
| 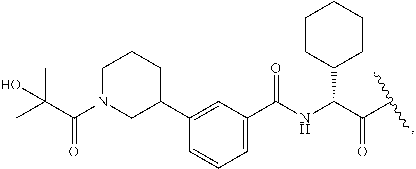 [SMILES: *C(=O)[C@H](NC(=O)c1cccc(C2CCCN(C(=O)C(C)(C)O)C2)c1)C1CCCCC1] | (vv) |
| 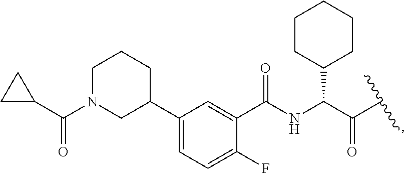 [SMILES: *C(=O)[C@H](NC(=O)c1cc(C2CCCN(C(=O)C3CC3)C2)ccc1F)C1CCCCC1] | (vw) |
| 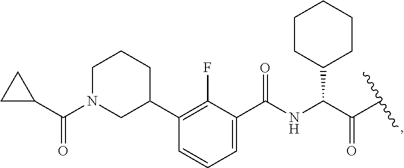 [SMILES: *C(=O)[C@H](NC(=O)c1cccc(C2CCCN(C(=O)C3CC3)C2)c1F)C1CCCCC1] | (vx) |
| 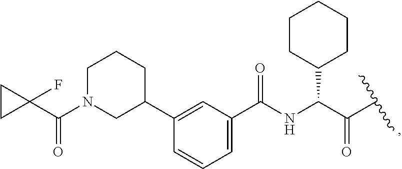 [SMILES: *C(=O)[C@H](NC(=O)c1cccc(C2CCCN(C(=O)C3(F)CC3)C2)c1)C1CCCCC1] | (vy) |
| 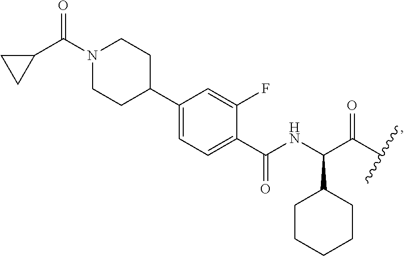 [SMILES: *C(=O)[C@H](NC(=O)c1ccc(C2CCN(C(=O)C3CC3)CC2)cc1F)C1CCCCC1] | (vz) |
| 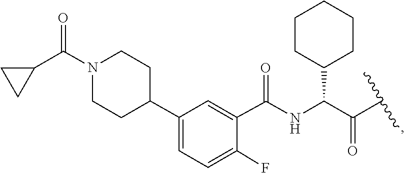 [SMILES: *C(=O)[C@H](NC(=O)c1cc(C2CCN(C(=O)C3CC3)CC2)ccc1F)C1CCCCC1] | (wa) |
| 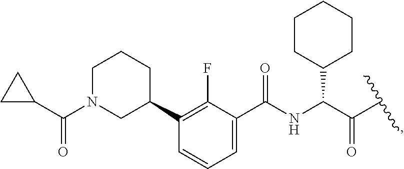 [SMILES: *C(=O)[C@H](NC(=O)c1cccc([C@@H]2CCCN(C(=O)C3CC3)C2)c1F)C1CCCCC1] | (wb) |
| 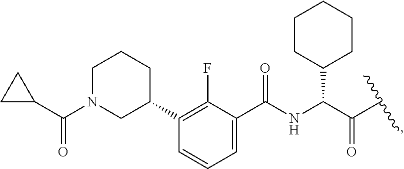 [SMILES: *C(=O)[C@H](NC(=O)c1cccc([C@H]2CCCN(C(=O)C3CC3)C2)c1F)C1CCCCC1] | (wc) |
| 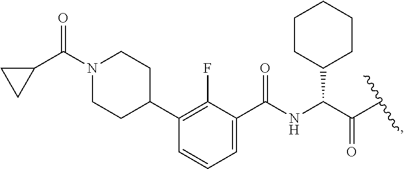 [SMILES: *C(=O)[C@H](NC(=O)c1cccc(C2CCN(C(=O)C3CC3)CC2)c1F)C1CCCCC1] | (wd) |
| 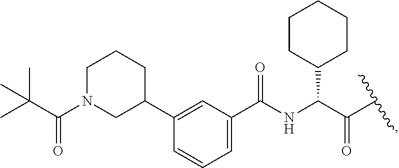 [SMILES: *C(=O)[C@H](NC(=O)c1cccc(C2CCCN(C(=O)C(C)(C)C)C2)c1)C1CCCCC1] | (we) |
| 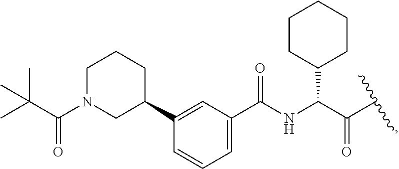 [SMILES: *C(=O)[C@H](NC(=O)c1cccc([C@@H]2CCCN(C(=O)C(C)(C)C)C2)c1)C1CCCCC1] | (wf) |
| 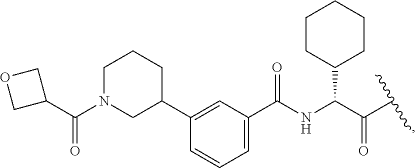 [SMILES: *C(=O)[C@H](NC(=O)c1cccc(C2CCCN(C(=O)C3COC3)C2)c1)C1CCCCC1] | (wg) |
| 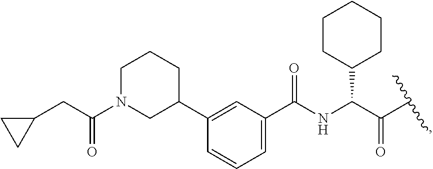 [SMILES: *C(=O)[C@H](NC(=O)c1cccc(C2CCCN(C(=O)CC3CC3)C2)c1)C1CCCCC1] | (wh) |
| 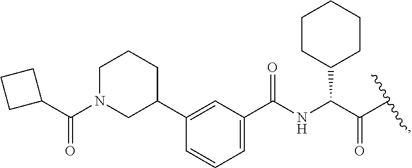 [SMILES: *C(=O)[C@H](NC(=O)c1cccc(C2CCCN(C(=O)C3CCC3)C2)c1)C1CCCCC1] | (wi) |
| 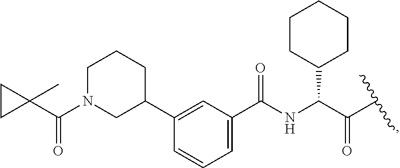 [SMILES: *C(=O)[C@H](NC(=O)c1cccc(C2CCCN(C(=O)C3(C)CC3)C2)c1)C1CCCCC1] | (wj) |
| 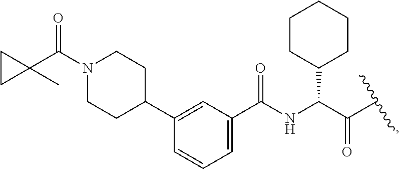 [SMILES: *C(=O)[C@H](NC(=O)c1cccc(C2CCN(C(=O)C3(C)CC3)CC2)c1)C1CCCCC1] | (wk) |
| 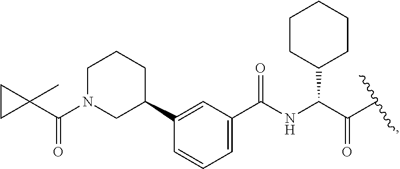 [SMILES: *C(=O)[C@H](NC(=O)c1cccc([C@@H]2CCCN(C(=O)C3(C)CC3)C2)c1)C1CCCCC1] | (wl) |
| 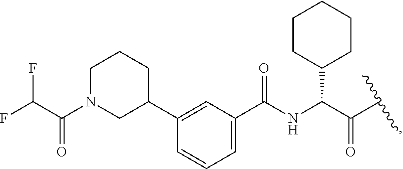 [SMILES: *C(=O)[C@H](NC(=O)c1cccc(C2CCCN(C(=O)C(F)F)C2)c1)C1CCCCC1] | (wm) |
| 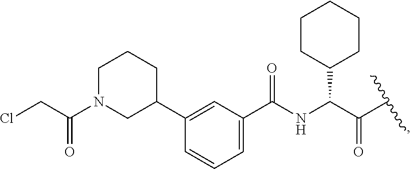 [SMILES: *C(=O)[C@H](NC(=O)c1cccc(C2CCCN(C(=O)CCl)C2)c1)C1CCCCC1] | (wn) |
| 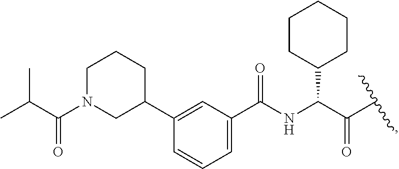 [SMILES: *C(=O)[C@H](NC(=O)c1cccc(C2CCCN(C(=O)C(C)C)C2)c1)C1CCCCC1] | (wo) |
| 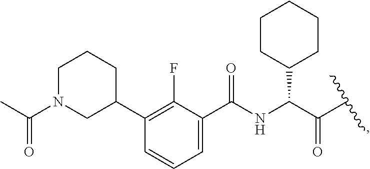 [SMILES: *C(=O)[C@H](NC(=O)c1cccc(C2CCCN(C(C)=O)C2)c1F)C1CCCCC1] | (wp) |
| 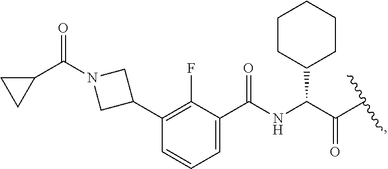 [SMILES: *C(=O)[C@H](NC(=O)c1cccc(C2CN(C(=O)C3CC3)C2)c1F)C1CCCCC1] | (wq) |
| 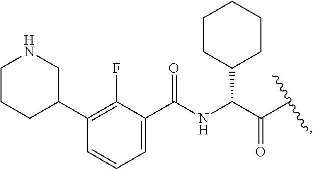 [SMILES: *C(=O)[C@H](NC(=O)c1cccc(C2CCCNC2)c1F)C1CCCCC1] | (wr) |
| 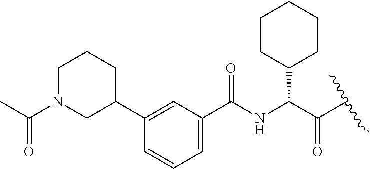 [SMILES: *C(=O)[C@H](NC(=O)c1cccc(C2CCCN(C(C)=O)C2)c1)C1CCCCC1] | (ws) |
| 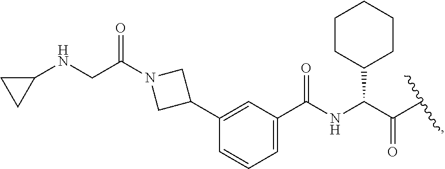 [SMILES: *C(=O)[C@H](NC(=O)c1cccc(C2CN(C(=O)CNC3CC3)C2)c1)C1CCCCC1] | (wt) |
| 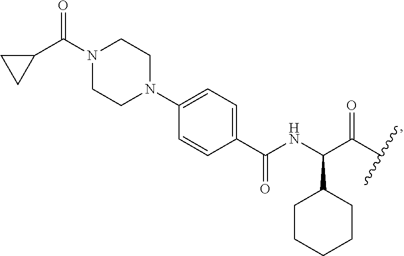 [SMILES: *C(=O)[C@H](NC(=O)c1ccc(N2CCN(C(=O)C3CC3)CC2)cc1)C1CCCCC1] | (wu) |
| 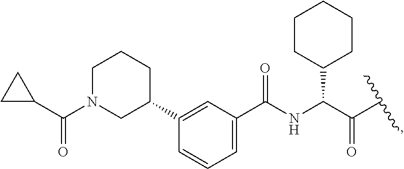 [SMILES: *C(=O)[C@H](NC(=O)c1cccc([C@H]2CCCN(C(=O)C3CC3)C2)c1)C1CCCCC1] | (wv) |
| 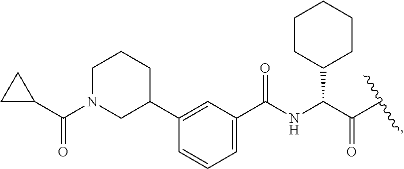 [SMILES: *C(=O)[C@H](NC(=O)c1cccc(C2CCCN(C(=O)C3CC3)C2)c1)C1CCCCC1] | (ww) |
| 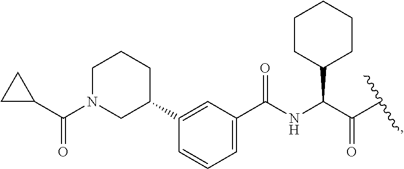 [SMILES: *C(=O)[C@@H](NC(=O)c1cccc([C@H]2CCCN(C(=O)C3CC3)C2)c1)C1CCCCC1] | (wx) |
| 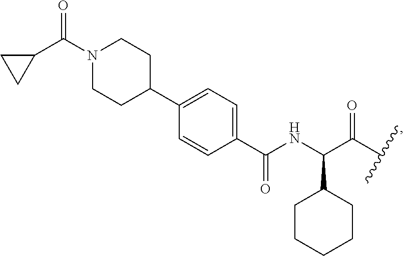 [SMILES: *C(=O)[C@H](NC(=O)c1ccc(C2CCN(C(=O)C3CC3)CC2)cc1)C1CCCCC1] | (wy) |
| 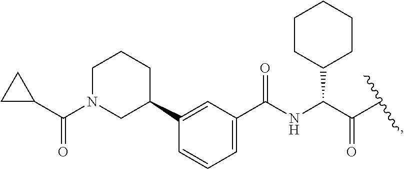 [SMILES: *C(=O)[C@H](NC(=O)c1cccc([C@@H]2CCCN(C(=O)C3CC3)C2)c1)C1CCCCC1] | (wz) |
| 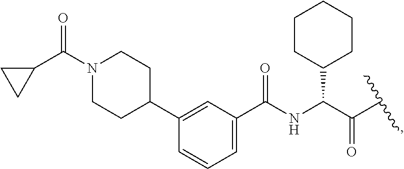 [SMILES: *C(=O)[C@H](NC(=O)c1cccc(C2CCN(C(=O)C3CC3)CC2)c1)C1CCCCC1] | (xa) |
| 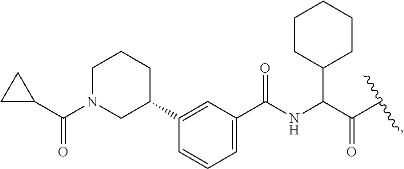 [SMILES: *C(=O)C(NC(=O)c1cccc([C@H]2CCCN(C(=O)C3CC3)C2)c1)C1CCCCC1] | (xb) |
| 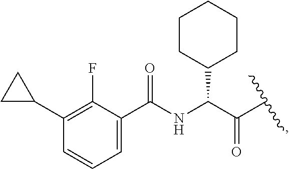 [SMILES: *C(=O)[C@H](NC(=O)c1cccc(C2CC2)c1F)C1CCCCC1] | (xc) |
| 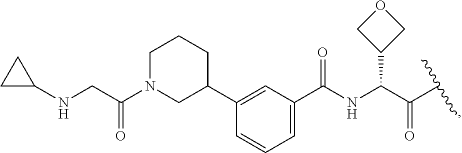 [SMILES: *C(=O)[C@H](NC(=O)c1cccc(C2CCCN(C(=O)CNC3CC3)C2)c1)C1COC1] | (xd) |
| 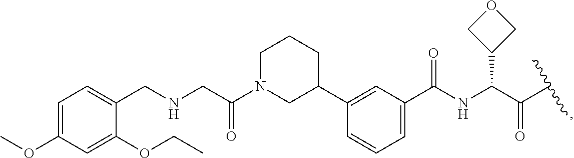 [SMILES: *C(=O)[C@H](NC(=O)c1cccc(C2CCCN(C(=O)CNCc3ccc(OC)cc3OCC)C2)c1)C1COC1] | (xe) |
| 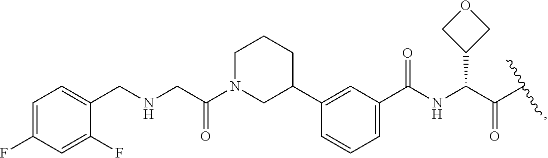 [SMILES: *C(=O)[C@H](NC(=O)c1cccc(C2CCCN(C(=O)CNCc3ccc(F)cc3F)C2)c1)C1COC1] | (xf) |
| 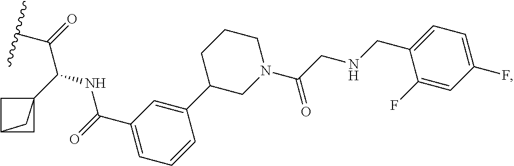 [SMILES: *C(=O)[C@H](NC(=O)c1cccc(C2CCCN(C(=O)CNCc3ccc(F)cc3F)C2)c1)C12CC(C1)C2] | (xg) |
| 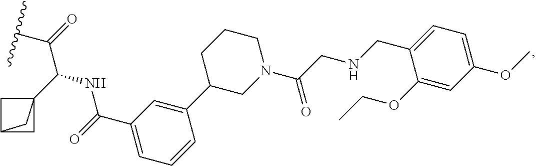 [SMILES: *C(=O)[C@H](NC(=O)c1cccc(C2CCCN(C(=O)CNCc3ccc(OC)cc3OCC)C2)c1)C12CC(C1)C2] | (xh) |
| 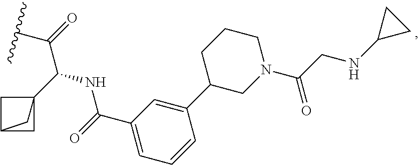 [SMILES: *C(=O)[C@H](NC(=O)c1cccc(C2CCCN(C(=O)CNC3CC3)C2)c1)C12CC(C1)C2] | (xi) |
| 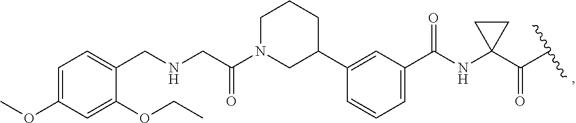 [SMILES: *C(=O)C1(NC(=O)c2cccc(C3CCCN(C(=O)CNCc4ccc(OC)cc4OCC)C3)c2)CC1] | (xj) |
| 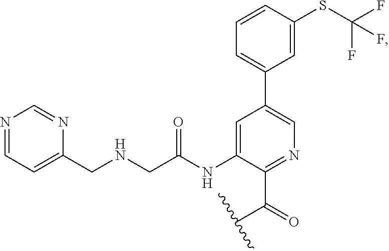 [SMILES: *C(=O)c1ncc(-c2cccc(SC(F)(F)F)c2)cc1NC(=O)CNCc1ccncn1] | (xk) |
| 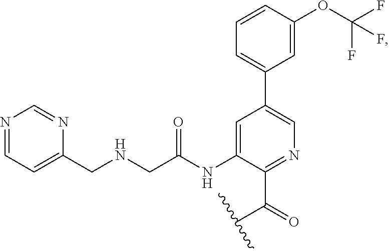 [SMILES: *C(=O)c1ncc(-c2cccc(OC(F)(F)F)c2)cc1NC(=O)CNCc1ccncn1] | (xl) |
| 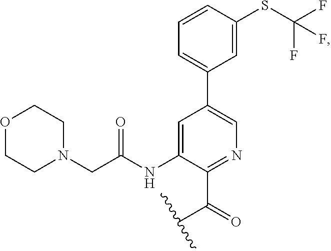 [SMILES: *C(=O)c1ncc(-c2cccc(SC(F)(F)F)c2)cc1NC(=O)CN1CCOCC1] | (xm) |
| 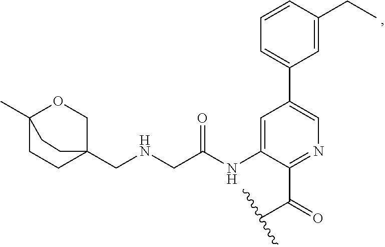 [SMILES: *C(=O)c1ncc(-c2cccc(CC)c2)cc1NC(=O)CNCC12CCC(C)(CC1)OC2] | (xn) |
| 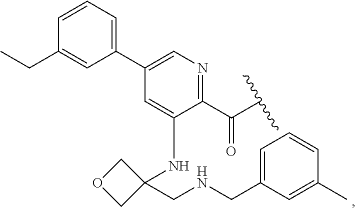 [SMILES: *C(=O)c1ncc(-c2cccc(CC)c2)cc1NC1(CNCc2cccc(C)c2)COC1] | (xo) |
| 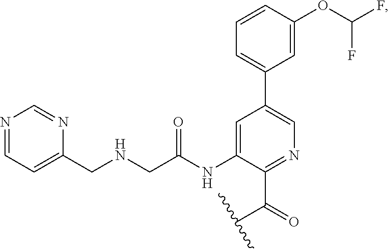 [SMILES: *C(=O)c1ncc(-c2cccc(OC(F)F)c2)cc1NC(=O)CNCc1ccncn1] | (xp) |
| 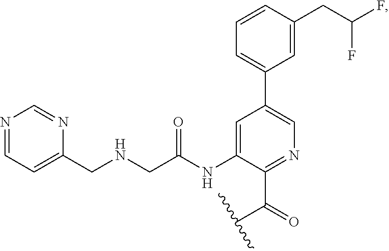 [SMILES: *C(=O)c1ncc(-c2cccc(CC(F)F)c2)cc1NC(=O)CNCc1ccncn1] | (xq) |
| 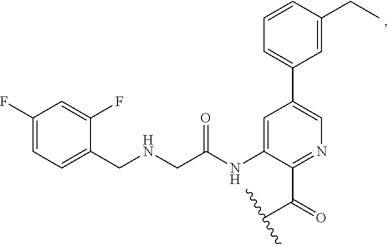 [SMILES: *C(=O)c1ncc(-c2cccc(CC)c2)cc1NC(=O)CNCc1ccc(F)cc1F] | (xr) |
| 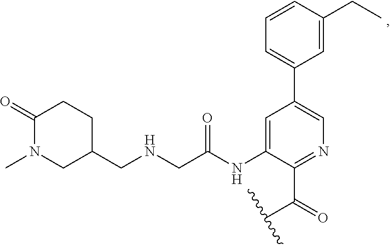 [SMILES: *C(=O)c1ncc(-c2cccc(CC)c2)cc1NC(=O)CNCC1CCC(=O)N(C)C1] | (xs) |
| 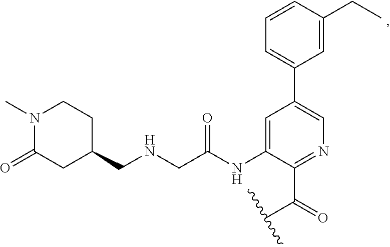 [SMILES: *C(=O)c1ncc(-c2cccc(CC)c2)cc1NC(=O)CNC[C@@H]1CCN(C)C(=O)C1] | (xt) |
| 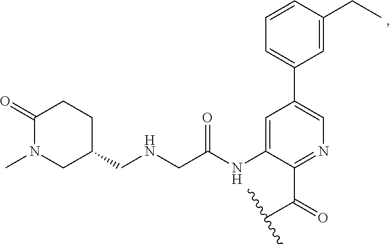 [SMILES: *C(=O)c1ncc(-c2cccc(CC)c2)cc1NC(=O)CNC[C@H]1CCC(=O)N(C)C1] | (xu) |
| 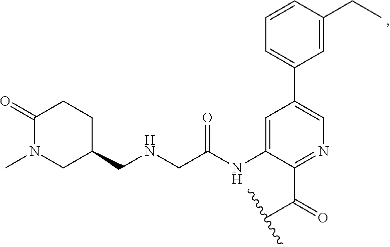 [SMILES: *C(=O)c1ncc(-c2cccc(CC)c2)cc1NC(=O)CNC[C@@H]1CCC(=O)N(C)C1] | (xv) |
| 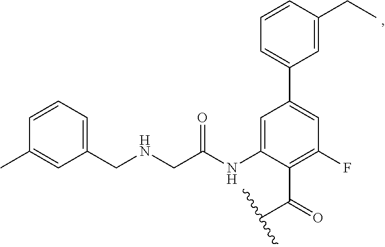 [SMILES: *C(=O)c1c(F)cc(-c2cccc(CC)c2)cc1NC(=O)CNCc1cccc(C)c1] | (xw) |
| 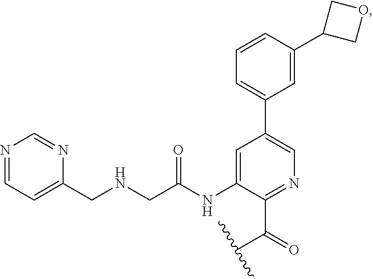 [SMILES: *C(=O)c1ncc(-c2cccc(C3COC3)c2)cc1NC(=O)CNCc1ccncn1] | (xx) |
| 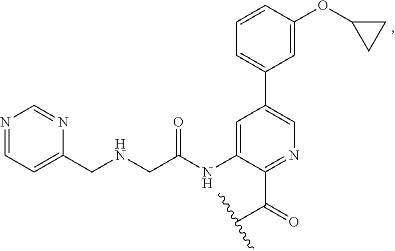 [SMILES: *C(=O)c1ncc(-c2cccc(OC3CC3)c2)cc1NC(=O)CNCc1ccncn1] | (xy) |
| 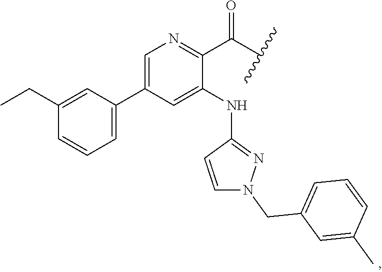 [SMILES: *C(=O)c1ncc(-c2cccc(CC)c2)cc1Nc1ccn(Cc2cccc(C)c2)n1] | (xz) |
| 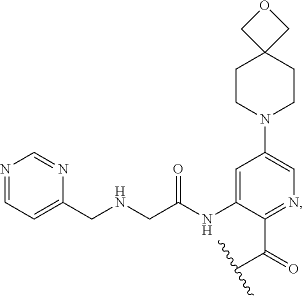 [SMILES: *C(=O)c1ncc(N2CCC3(CC2)COC3)cc1NC(=O)CNCc1ccncn1] | (ya) |
| 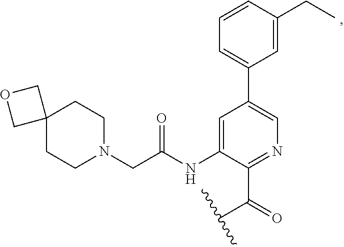 [SMILES: *C(=O)c1ncc(-c2cccc(CC)c2)cc1NC(=O)CN1CCC2(CC1)COC2] | (yb) |
| 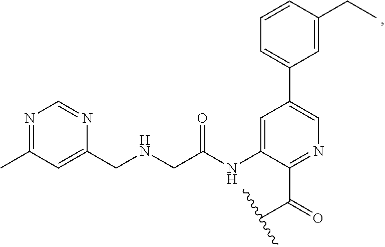 [SMILES: *C(=O)c1ncc(-c2cccc(CC)c2)cc1NC(=O)CNCc1cc(C)ncn1] | (yc) |
| 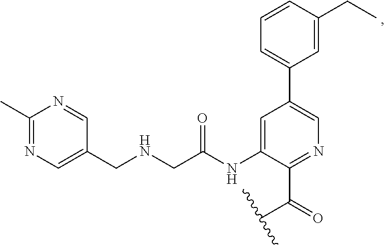 [SMILES: *C(=O)c1ncc(-c2cccc(CC)c2)cc1NC(=O)CNCc1cnc(C)nc1] | (yd) |
| 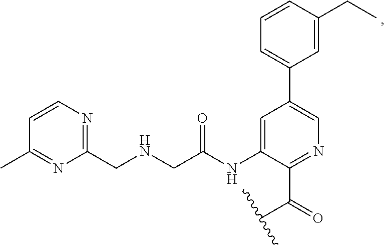 [SMILES: *C(=O)c1ncc(-c2cccc(CC)c2)cc1NC(=O)CNCc1nccc(C)n1] | (ye) |
| 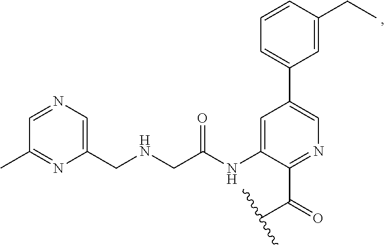 [SMILES: *C(=O)c1ncc(-c2cccc(CC)c2)cc1NC(=O)CNCc1cncc(C)n1] | (yf) |
| 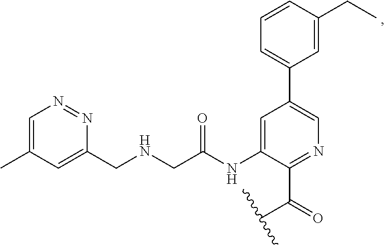 [SMILES: *C(=O)c1ncc(-c2cccc(CC)c2)cc1NC(=O)CNCc1cc(C)cnn1] | (yg) |
| 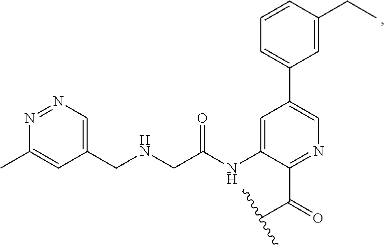 [SMILES: *C(=O)c1ncc(-c2cccc(CC)c2)cc1NC(=O)CNCc1cnnc(C)c1] | (yh) |
| 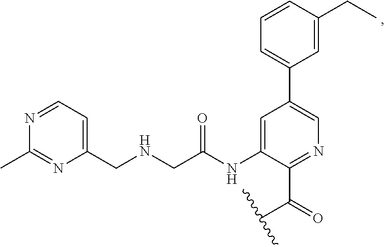 [SMILES: *C(=O)c1ncc(-c2cccc(CC)c2)cc1NC(=O)CNCc1ccnc(C)n1] | (yi) |
| 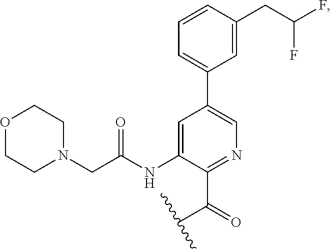 [SMILES: *C(=O)c1ncc(-c2cccc(CC(F)F)c2)cc1NC(=O)CN1CCOCC1] | (yj) |
| 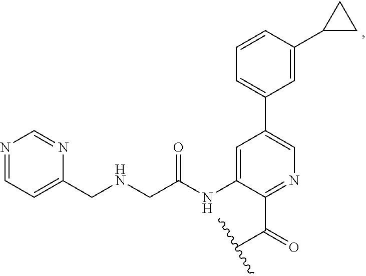 [SMILES: *C(=O)c1ncc(-c2cccc(C3CC3)c2)cc1NC(=O)CNCc1ccncn1] | (yk) |
| 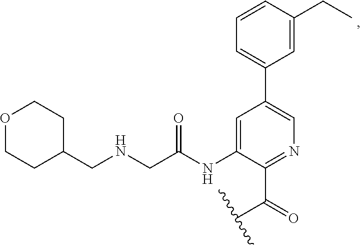 [SMILES: *C(=O)c1ncc(-c2cccc(CC)c2)cc1NC(=O)CNCC1CCOCC1] | (yl) |
| 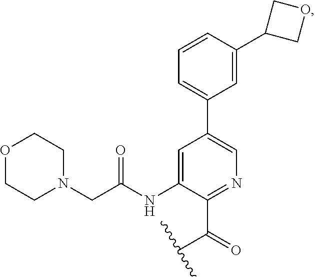 [SMILES: *C(=O)c1ncc(-c2cccc(C3COC3)c2)cc1NC(=O)CN1CCOCC1] | (ym) |
| 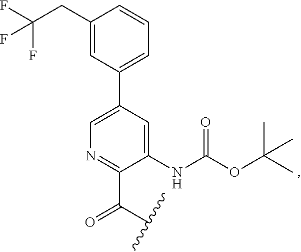 [SMILES: *C(=O)c1ncc(-c2cccc(CC(F)(F)F)c2)cc1NC(=O)OC(C)(C)C] | (yn) |
| 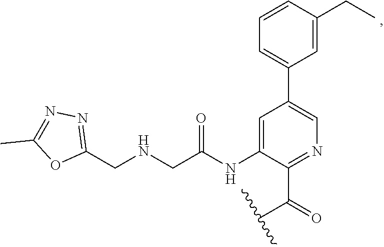 [SMILES: *C(=O)c1ncc(-c2cccc(CC)c2)cc1NC(=O)CNCc1nnc(C)o1] | (yo) |
| 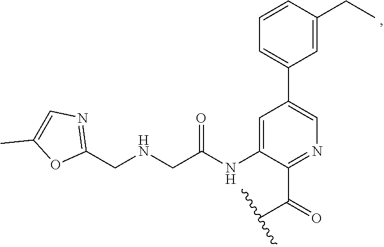 [SMILES: *C(=O)c1ncc(-c2cccc(CC)c2)cc1NC(=O)CNCc1ncc(C)o1] | (yp) |
| 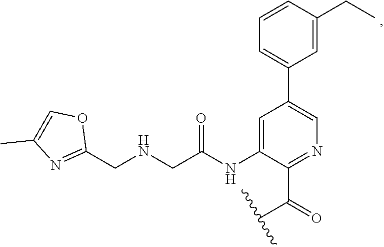 [SMILES: *C(=O)c1ncc(-c2cccc(CC)c2)cc1NC(=O)CNCc1nc(C)co1] | (yq) |
| 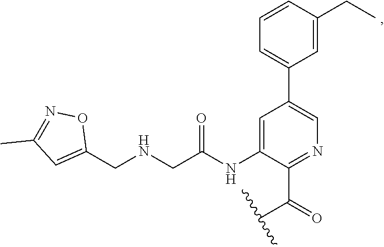 [SMILES: *C(=O)c1ncc(-c2cccc(CC)c2)cc1NC(=O)CNCc1cc(C)no1] | (yr) |
| 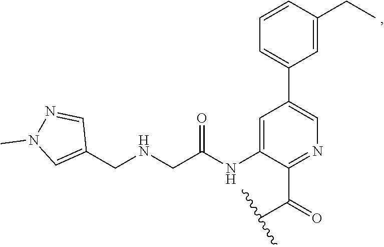 [SMILES: *C(=O)c1ncc(-c2cccc(CC)c2)cc1NC(=O)CNCc1cnn(C)c1] | (ys) |
| 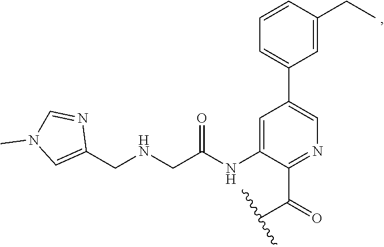 [SMILES: *C(=O)c1ncc(-c2cccc(CC)c2)cc1NC(=O)CNCc1cn(C)cn1] | (yt) |
| 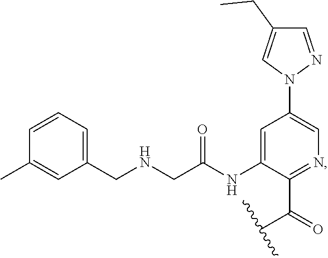 [SMILES: *C(=O)c1ncc(-n2cc(CC)cn2)cc1NC(=O)CNCc1cccc(C)c1] | (yu) |
| 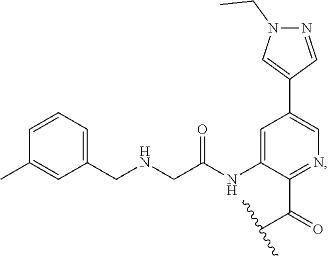 [SMILES: *C(=O)c1ncc(-c2cnn(CC)c2)cc1NC(=O)CNCc1cccc(C)c1] | (yv) |
| 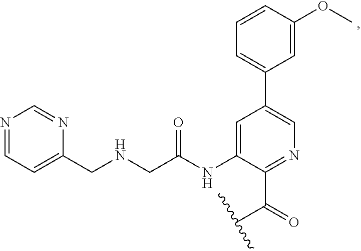 [SMILES: *C(=O)c1ncc(-c2cccc(OC)c2)cc1NC(=O)CNCc1ccncn1] | (yw) |
| 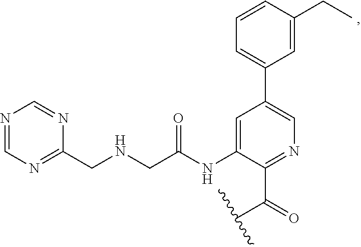 [SMILES: *C(=O)c1ncc(-c2cccc(CC)c2)cc1NC(=O)CNCc1ncncn1] | (yx) |
| 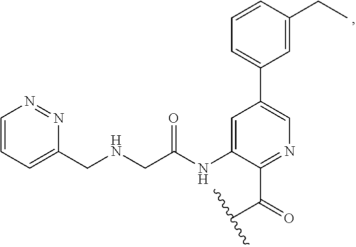 [SMILES: *C(=O)c1ncc(-c2cccc(CC)c2)cc1NC(=O)CNCc1cccnn1] | (yy) |
| 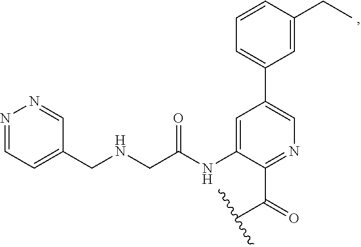 [SMILES: *C(=O)c1ncc(-c2cccc(CC)c2)cc1NC(=O)CNCc1ccnnc1] | (yz) |
| 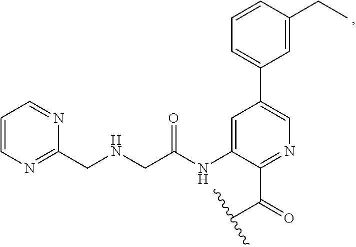 [SMILES: *C(=O)c1ncc(-c2cccc(CC)c2)cc1NC(=O)CNCc1ncccn1] | (za) |
| 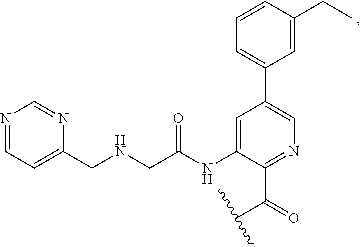 [SMILES: *C(=O)c1ncc(-c2cccc(CC)c2)cc1NC(=O)CNCc1ccncn1] | (zb) |
| 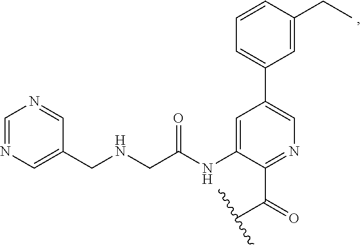 [SMILES: *C(=O)c1ncc(-c2cccc(CC)c2)cc1NC(=O)CNCc1cncnc1] | (zc) |
| 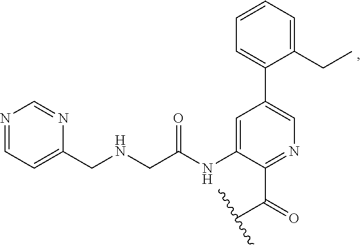 [SMILES: *C(=O)c1ncc(-c2ccccc2CC)cc1NC(=O)CNCc1ccncn1] | (zd) |
| 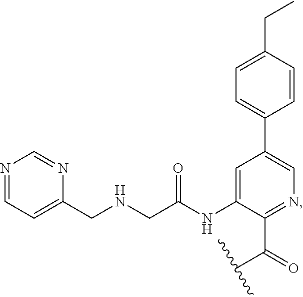 [SMILES: *C(=O)c1ncc(-c2ccc(CC)cc2)cc1NC(=O)CNCc1ccncn1] | (ze) |
| 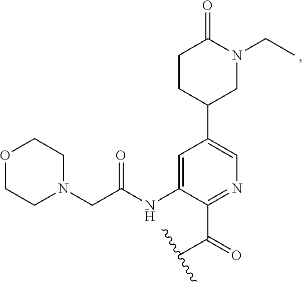 [SMILES: *C(=O)c1ncc(C2CCC(=O)N(CC)C2)cc1NC(=O)CN1CCOCC1] | (zf) |
| 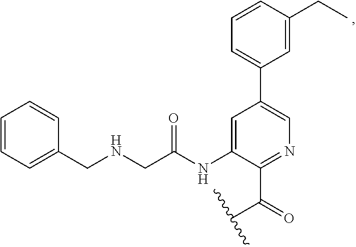 [SMILES: *C(=O)c1ncc(-c2cccc(CC)c2)cc1NC(=O)CNCc1ccccc1] | (zg) |
| 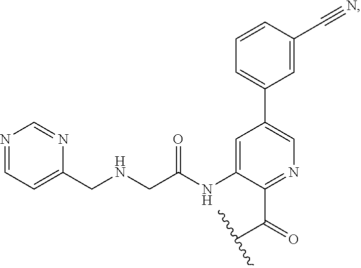 [SMILES: *C(=O)c1ncc(-c2cccc(C#N)c2)cc1NC(=O)CNCc1ccncn1] | (zh) |
| 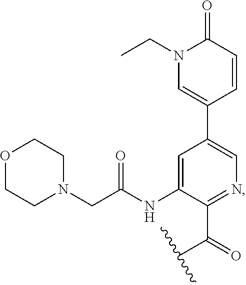 [SMILES: *C(=O)c1ncc(-c2ccc(=O)n(CC)c2)cc1NC(=O)CN1CCOCC1] | (zi) |
| 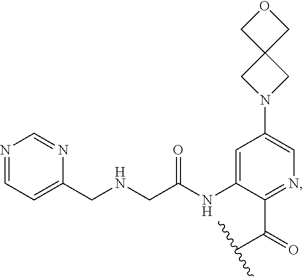 [SMILES: *C(=O)c1ncc(N2CC3(COC3)C2)cc1NC(=O)CNCc1ccncn1] | (zj) |
| 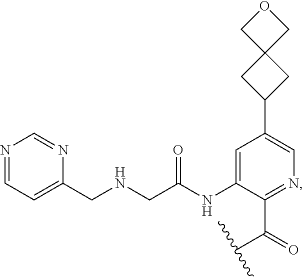 [SMILES: *C(=O)c1ncc(C2CC3(COC3)C2)cc1NC(=O)CNCc1ccncn1] | (zk) |
| 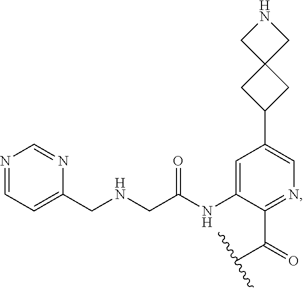 [SMILES: *C(=O)c1ncc(C2CC3(CNC3)C2)cc1NC(=O)CNCc1ccncn1] | (zl) |
| 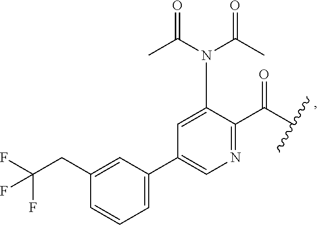 [SMILES: *C(=O)c1ncc(-c2cccc(CC(F)(F)F)c2)cc1N(C(C)=O)C(C)=O] | (zm) |
| 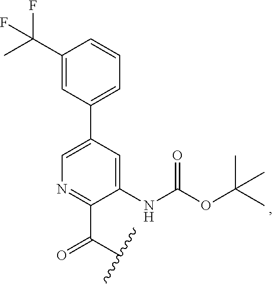 [SMILES: *C(=O)c1ncc(-c2cccc(C(C)(F)F)c2)cc1NC(=O)OC(C)(C)C] | (zn) |
| 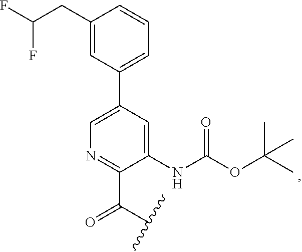 [SMILES: *C(=O)c1ncc(-c2cccc(CC(F)F)c2)cc1NC(=O)OC(C)(C)C] | (zo) |
| 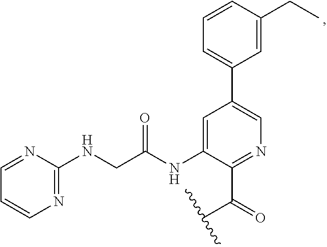 [SMILES: *C(=O)c1ncc(-c2cccc(CC)c2)cc1NC(=O)CNc1ncccn1] | (zp) |
| 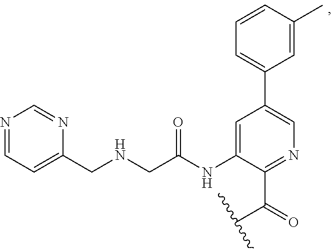 [SMILES: *C(=O)c1ncc(-c2cccc(C)c2)cc1NC(=O)CNCc1ccncn1] | (zq) |
| 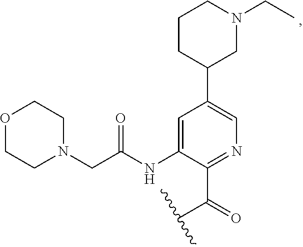 [SMILES: *C(=O)c1ncc(C2CCCN(CC)C2)cc1NC(=O)CN1CCOCC1] | (zr) |
| 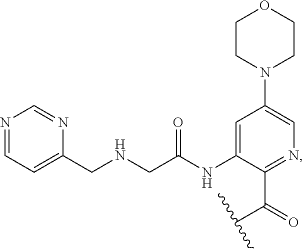 [SMILES: *C(=O)c1ncc(N2CCOCC2)cc1NC(=O)CNCc1ccncn1] | (zs) |
| 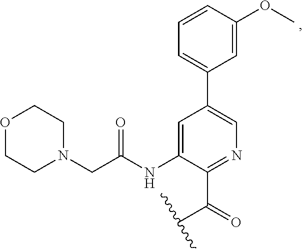 [SMILES: *C(=O)c1ncc(-c2cccc(OC)c2)cc1NC(=O)CN1CCOCC1] | (zt) |
| 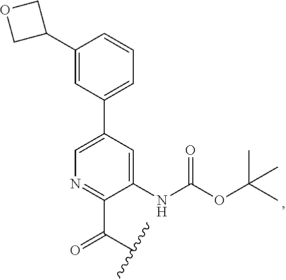 [SMILES: *C(=O)c1ncc(-c2cccc(C3COC3)c2)cc1NC(=O)OC(C)(C)C] | (zu) |
| 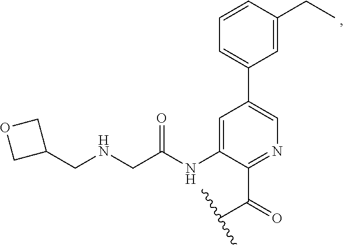 [SMILES: *C(=O)c1ncc(-c2cccc(CC)c2)cc1NC(=O)CNCC1COC1] | (zv) |
| 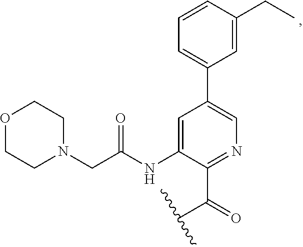 [SMILES: *C(=O)c1ncc(-c2cccc(CC)c2)cc1NC(=O)CN1CCOCC1] | (zw) |
| 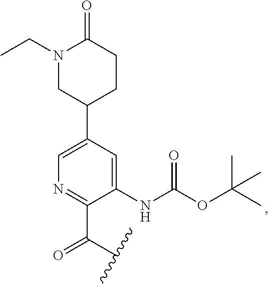 [SMILES: *C(=O)c1ncc(C2CCC(=O)N(CC)C2)cc1NC(=O)OC(C)(C)C] | (zx) |
| 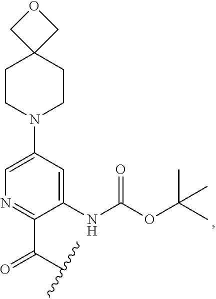 [SMILES: *C(=O)c1ncc(N2CCC3(CC2)COC3)cc1NC(=O)OC(C)(C)C] | (zy) |
| 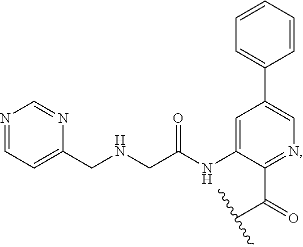 [SMILES: *C(=O)c1ncc(-c2ccccc2)cc1NC(=O)CNCc1ccncn1] | (zz) |
| 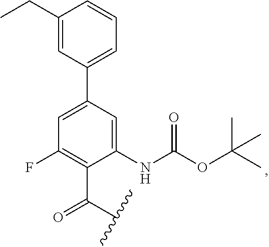 [SMILES: *C(=O)c1c(F)cc(-c2cccc(CC)c2)cc1NC(=O)OC(C)(C)C] | (aaa) |
| 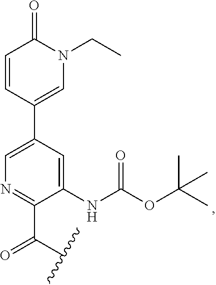 [SMILES: *C(=O)c1ncc(-c2ccc(=O)n(CC)c2)cc1NC(=O)OC(C)(C)C] | (aab) |
| 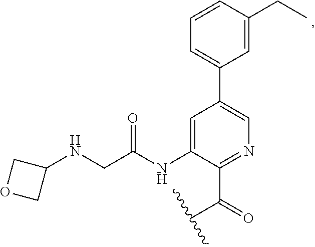 [SMILES: *C(=O)c1ncc(-c2cccc(CC)c2)cc1NC(=O)CNC1COC1] | (aac) |
| 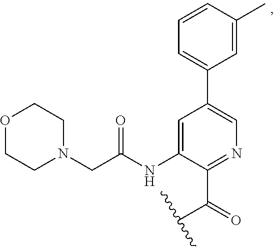 [SMILES: *C(=O)c1ncc(-c2cccc(C)c2)cc1NC(=O)CN1CCOCC1] | (aad) |
| 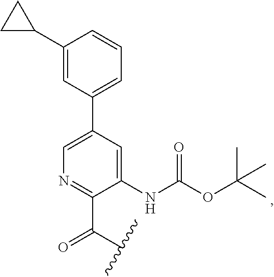 [SMILES: *C(=O)c1ncc(-c2cccc(C3CC3)c2)cc1NC(=O)OC(C)(C)C] | (aae) |
| 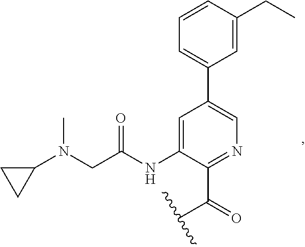 [SMILES: *C(=O)c1ncc(-c2cccc(CC)c2)cc1NC(=O)CN(C)C1CC1] | (aaf) |
| 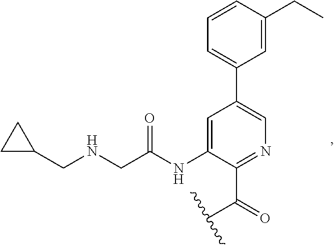 [SMILES: *C(=O)c1ncc(-c2cccc(CC)c2)cc1NC(=O)CNCC1CC1] | (aag) |
| 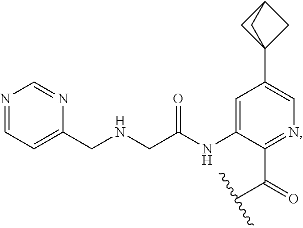 [SMILES: *C(=O)c1ncc(C23CC(C2)C3)cc1NC(=O)CNCc1ccncn1] | (aah) |
| 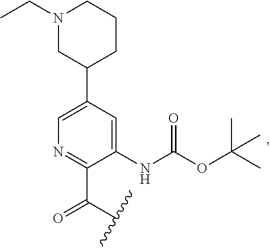 [SMILES: *C(=O)c1ncc(C2CCCN(CC)C2)cc1NC(=O)OC(C)(C)C] | (aai) |
| 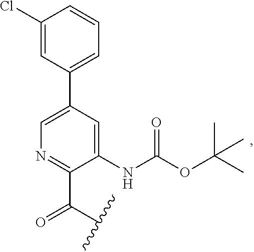 [SMILES: *C(=O)c1ncc(-c2cccc(Cl)c2)cc1NC(=O)OC(C)(C)C] | (aaj) |
| 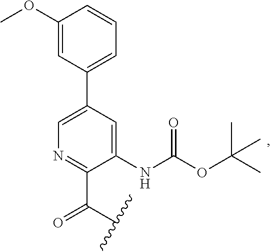 [SMILES: *C(=O)c1ncc(-c2cccc(OC)c2)cc1NC(=O)OC(C)(C)C] | (aak) |
| 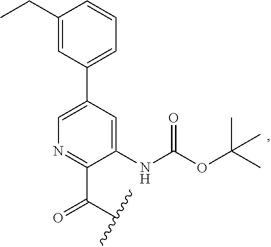 [SMILES: *C(=O)c1ncc(-c2cccc(CC)c2)cc1NC(=O)OC(C)(C)C] | (aal) |
| 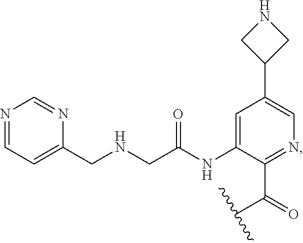 [SMILES: *C(=O)c1ncc(C2CNC2)cc1NC(=O)CNCc1ccncn1] | (aam) |
| 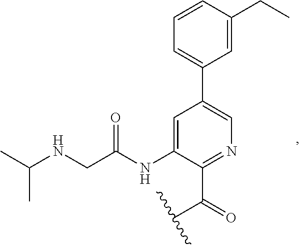 [SMILES: *C(=O)c1ncc(-c2cccc(CC)c2)cc1NC(=O)CNC(C)C] | (aan) |
| 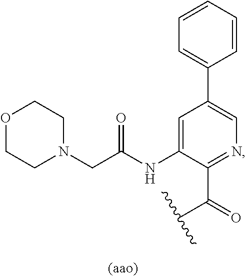 [SMILES: *C(=O)c1ncc(-c2ccccc2)cc1NC(=O)CN1CCOCC1] | (aao) |
| 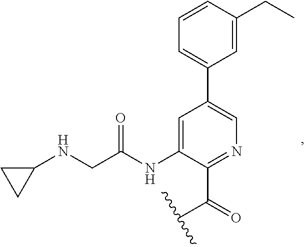 [SMILES: *C(=O)c1ncc(-c2cccc(CC)c2)cc1NC(=O)CNC1CC1] | (aap) |
| 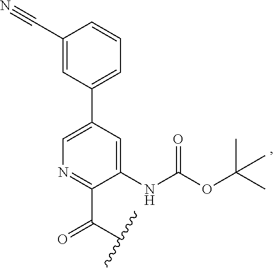 [SMILES: *C(=O)c1ncc(-c2cccc(C#N)c2)cc1NC(=O)OC(C)(C)C] | (aaq) |
| 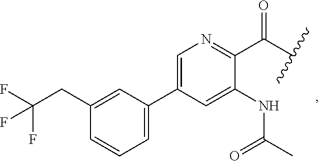 [SMILES: *C(=O)c1ncc(-c2cccc(CC(F)(F)F)c2)cc1NC(C)=O] | (aar) |
| 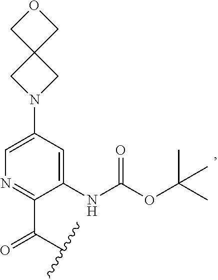 [SMILES: *C(=O)c1ncc(N2CC3(COC3)C2)cc1NC(=O)OC(C)(C)C] | (aas) |
| 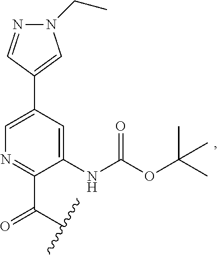 [SMILES: *C(=O)c1ncc(-c2cnn(CC)c2)cc1NC(=O)OC(C)(C)C] | (aat) |
| 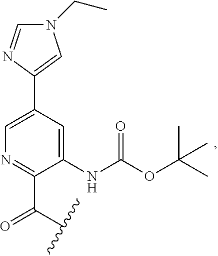 [SMILES: *C(=O)c1ncc(-c2cn(CC)cn2)cc1NC(=O)OC(C)(C)C] | (aau) |
| 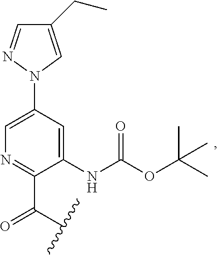 [SMILES: *C(=O)c1ncc(-n2cc(CC)cn2)cc1NC(=O)OC(C)(C)C] | (aav) |
| 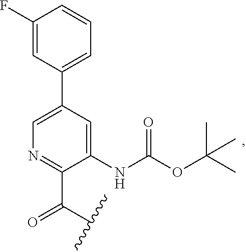 [SMILES: *C(=O)c1ncc(-c2cccc(F)c2)cc1NC(=O)OC(C)(C)C] | (aaw) |
| 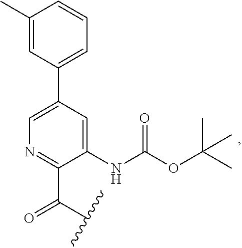 [SMILES: *C(=O)c1ncc(-c2cccc(C)c2)cc1NC(=O)OC(C)(C)C] | (aax) |
| 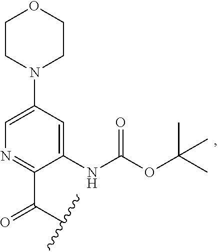 [SMILES: *C(=O)c1ncc(N2CCOCC2)cc1NC(=O)OC(C)(C)C] | (aay) |
| 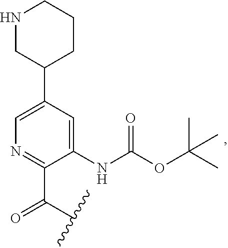 [SMILES: *C(=O)c1ncc(C2CCCNC2)cc1NC(=O)OC(C)(C)C] | (aaz) |
| 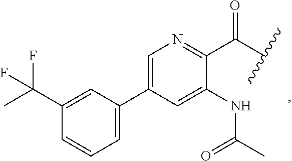 [SMILES: *C(=O)c1ncc(-c2cccc(C(C)(F)F)c2)cc1NC(C)=O] | (aba) |
| 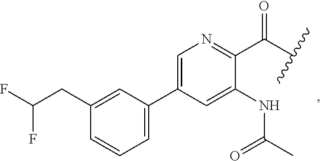 [SMILES: *C(=O)c1ncc(-c2cccc(CC(F)F)c2)cc1NC(C)=O] | (abb) |
| 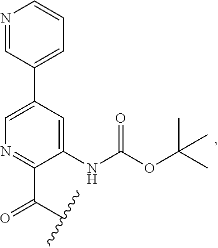 [SMILES: *C(=O)c1ncc(-c2cccnc2)cc1NC(=O)OC(C)(C)C] | (abc) |
| 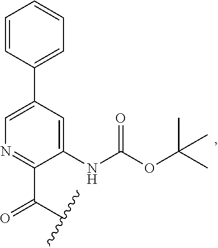 [SMILES: *C(=O)c1ncc(-c2ccccc2)cc1NC(=O)OC(C)(C)C] | (abd) |
| 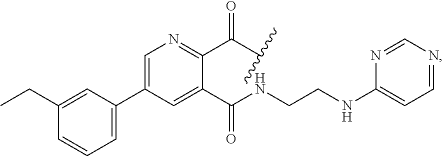 [SMILES: *C(=O)c1ncc(-c2cccc(CC)c2)cc1C(=O)NCCNc1ccncn1] | (abe) |
| 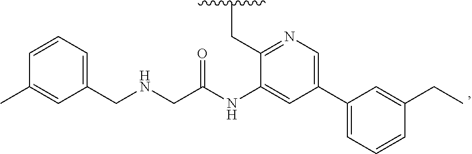 [SMILES: *Cc1ncc(-c2cccc(CC)c2)cc1NC(=O)CNCc1cccc(C)c1] | (abf) |
| 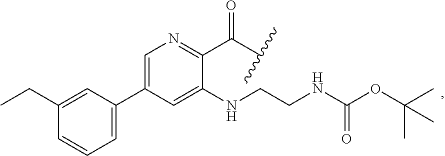 [SMILES: *C(=O)c1ncc(-c2cccc(CC)c2)cc1NCCNC(=O)OC(C)(C)C] | (abg) |
| 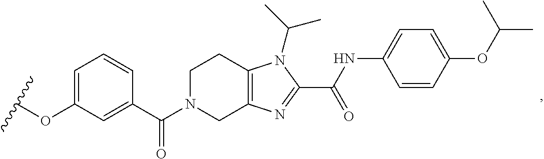 [SMILES: *Oc1cccc(C(=O)N2CCc3c(nc(C(=O)Nc4ccc(OC(C)C)cc4)n3C(C)C)C2)c1] | (abh) |
| 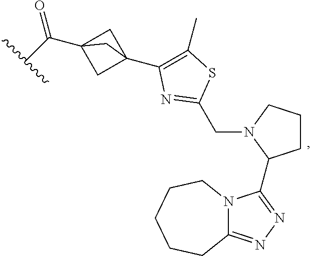 [SMILES: *C(=O)C12CC(c3nc(CN4CCCC4c4nnc5n4CCCCC5)sc3C)(C1)C2] | (abi) |
| 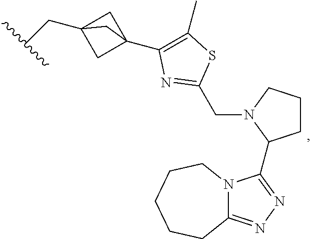 [SMILES: *CC12CC(c3nc(CN4CCCC4c4nnc5n4CCCCC5)sc3C)(C1)C2] | (abj) |
| 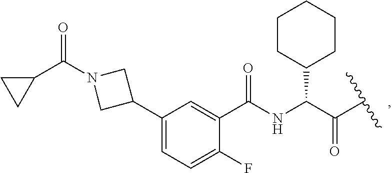 [SMILES: *C(=O)[C@H](NC(=O)c1cc(C2CN(C(=O)C3CC3)C2)ccc1F)C1CCCCC1] | (abd) |
| 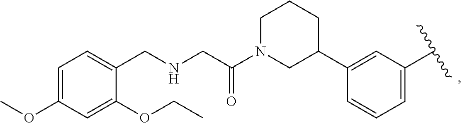 [SMILES: *c1cccc(C2CCCN(C(=O)CNCc3ccc(OC)cc3OCC)C2)c1] | (abl) |
| 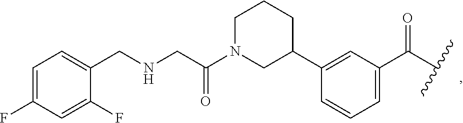 [SMILES: *C(=O)c1cccc(C2CCCN(C(=O)CNCc3ccc(F)cc3F)C2)c1] | (abm) |
| 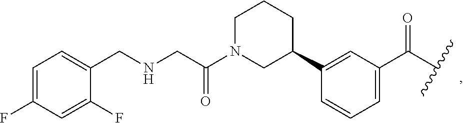 [SMILES: *C(=O)c1cccc([C@@H]2CCCN(C(=O)CNCc3ccc(F)cc3F)C2)c1] | (abn) |
| 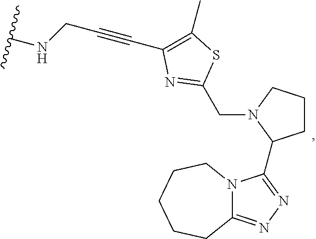 [SMILES: *NCC#Cc1nc(CN2CCCC2c2nnc3n2CCCCC3)sc1C] | (abo) |
| 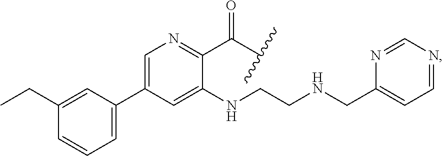 [SMILES: *C(=O)c1ncc(-c2cccc(CC)c2)cc1NCCNCc1ccncn1] | (abp) |
| 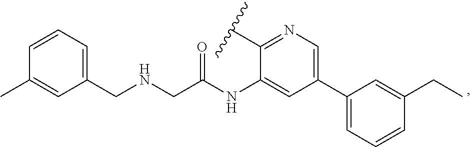 [SMILES: *c1ncc(-c2cccc(CC)c2)cc1NC(=O)CNCc1cccc(C)c1] | (abq) |
| 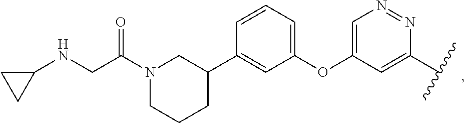 [SMILES: *c1cc(Oc2cccc(C3CCCN(C(=O)CNC4CC4)C3)c2)cnn1] | (abr) |
| 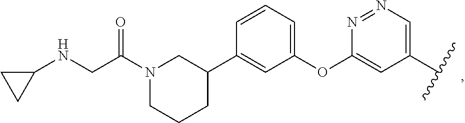 [SMILES: *c1cnnc(Oc2cccc(C3CCCN(C(=O)CNC4CC4)C3)c2)c1] | (abs) |
| 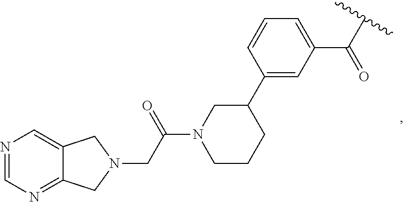 [SMILES: *C(=O)c1cccc(C2CCCN(C(=O)CN3Cc4cncnc4C3)C2)c1] | (abt) |
| 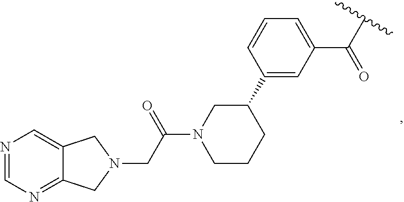 [SMILES: *C(=O)c1cccc([C@@H]2CCCN(C(=O)CN3Cc4cncnc4C3)C2)c1] | (abu) |
| 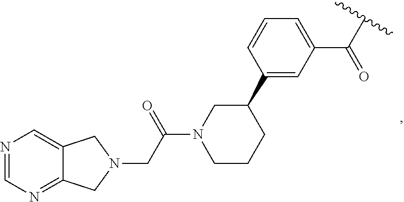 [SMILES: *C(=O)c1cccc([C@H]2CCCN(C(=O)CN3Cc4cncnc4C3)C2)c1] | (abv) |
| 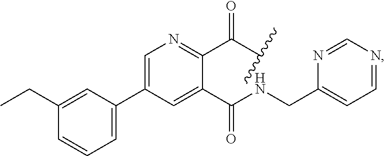 [SMILES: *C(=O)c1ncc(-c2cccc(CC)c2)cc1C(=O)NCc1ccncn1] | (abw) |
| 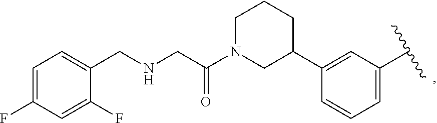 [SMILES: *c1cccc(C2CCCN(C(=O)CNCc3ccc(F)cc3F)C2)c1] | (abx) |
| 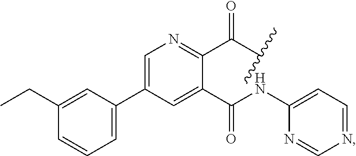 [SMILES: *C(=O)c1ncc(-c2cccc(CC)c2)cc1C(=O)Nc1ccncn1] | (aby) |
| 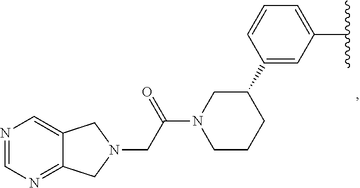 [SMILES: *c1cccc([C@@H]2CCCN(C(=O)CN3Cc4cncnc4C3)C2)c1] | (abz) |
| 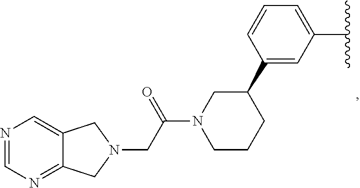 [SMILES: *c1cccc([C@H]2CCCN(C(=O)CN3Cc4cncnc4C3)C2)c1] | (aca) |
| 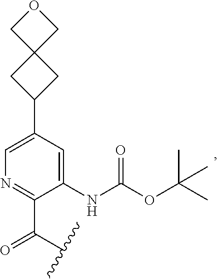 [SMILES: *C(=O)c1ncc(C2CC3(COC3)C2)cc1NC(=O)OC(C)(C)C] | (acb) |
| 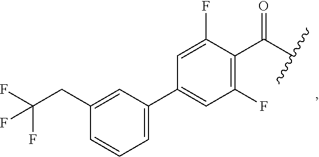 [SMILES: *C(=O)c1c(F)cc(-c2cccc(CC(F)(F)F)c2)cc1F] | (acc) |
| 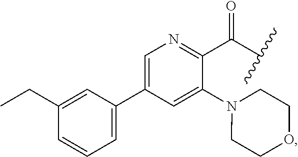 [SMILES: *C(=O)c1ncc(-c2cccc(CC)c2)cc1N1CCOCC1] | (acd) |
| 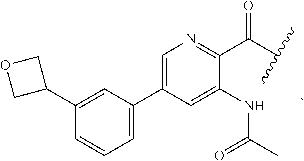 [SMILES: *C(=O)c1ncc(-c2cccc(C3COC3)c2)cc1NC(C)=O] | (ace) |
| 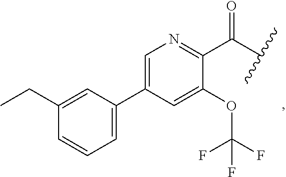 [SMILES: *C(=O)c1ncc(-c2cccc(CC)c2)cc1OC(F)(F)F] | (acf) |
| 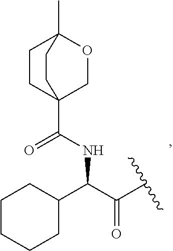 [SMILES: *C(=O)[C@H](NC(=O)C12CCC(C)(CC1)OC2)C1CCCCC1] | (acg) |
| 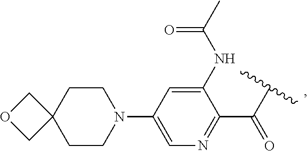 [SMILES: *C(=O)c1ncc(N2CCC3(CC2)COC3)cc1NC(C)=O] | (ach) |
| 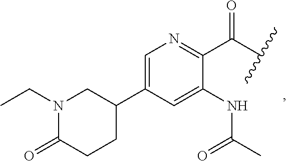 [SMILES: *C(=O)c1ncc(C2CCC(=O)N(CC)C2)cc1NC(C)=O] | (aci) |
| 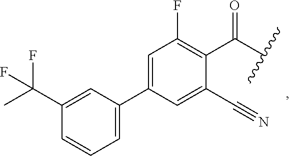 [SMILES: *C(=O)c1c(F)cc(-c2cccc(C(C)(F)F)c2)cc1C#N] | (acj) |
| 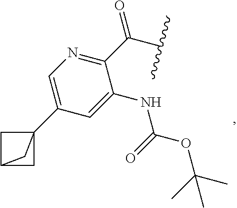 [SMILES: *C(=O)c1ncc(C23CC(C2)C3)cc1NC(=O)OC(C)(C)C] | (ack) |
| 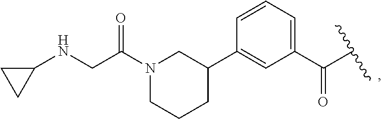 [SMILES: *C(=O)c1cccc(C2CCCN(C(=O)CNC3CC3)C2)c1] | (acl) |
| 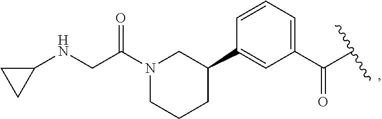 [SMILES: *C(=O)c1cccc([C@H]2CCCN(C(=O)CNC3CC3)C2)c1] | (acm) |
| 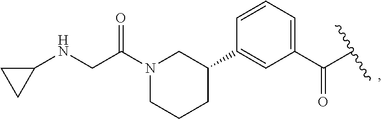 [SMILES: *C(=O)c1cccc([C@@H]2CCCN(C(=O)CNC3CC3)C2)c1] | (acn) |
| 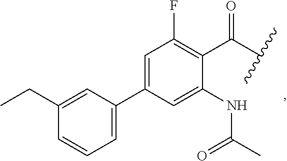 [SMILES: *C(=O)c1c(F)cc(-c2cccc(CC)c2)cc1NC(C)=O] | (aco) |
| 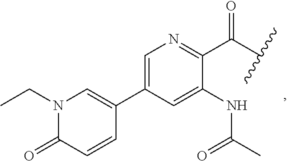 [SMILES: *C(=O)c1ncc(-c2ccc(=O)n(CC)c2)cc1NC(C)=O] | (acp) |
| 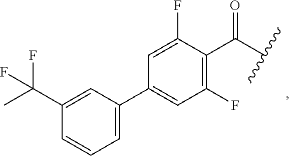 [SMILES: *C(=O)c1c(F)cc(-c2cccc(C(C)(F)F)c2)cc1F] | (acq) |
| 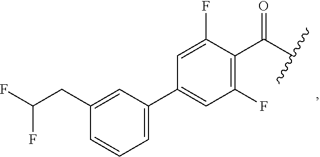 [SMILES: *C(=O)c1c(F)cc(-c2cccc(CC(F)F)c2)cc1F] | (acr) |
| 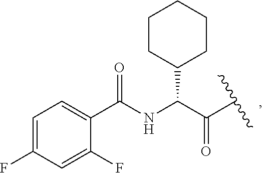 [SMILES: *C(=O)[C@H](NC(=O)c1ccc(F)cc1F)C1CCCCC1] | (acs) |
| 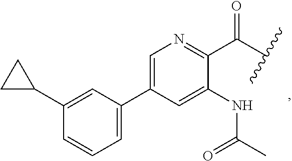 [SMILES: *C(=O)c1ncc(-c2cccc(C3CC3)c2)cc1NC(C)=O] | (act) |
| 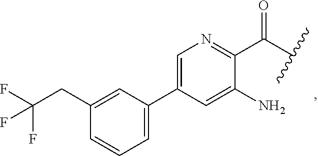 [SMILES: *C(=O)c1ncc(-c2cccc(CC(F)(F)F)c2)cc1N] | (acu) |
| 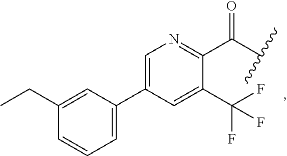 [SMILES: *C(=O)c1ncc(-c2cccc(CC)c2)cc1C(F)(F)F] | (acv) |
| 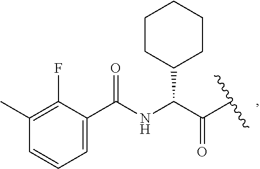 [SMILES: *C(=O)[C@H](NC(=O)c1cccc(C)c1F)C1CCCCC1] | (acw) |
| 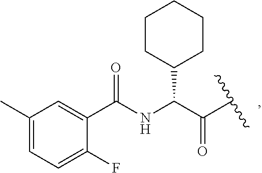 [SMILES: *C(=O)[C@H](NC(=O)c1cc(C)ccc1F)C1CCCCC1] | (acx) |
| 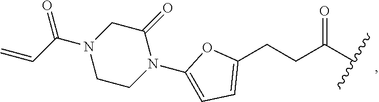 [SMILES: *C(=O)CCc1ccc(N2CCN(C(=O)C=C)CC2=O)o1] | (acy) |
| 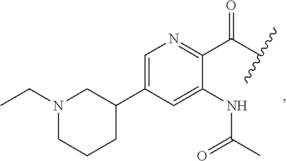 [SMILES: *C(=O)c1ncc(C2CCCN(CC)C2)cc1NC(C)=O] | (acz) |
| 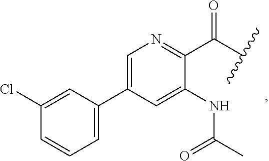 [SMILES: *C(=O)c1ncc(-c2cccc(Cl)c2)cc1NC(C)=O] | (ada) |
| 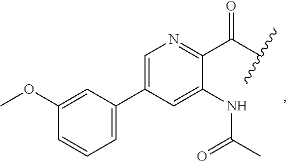 [SMILES: *C(=O)c1ncc(-c2cccc(OC)c2)cc1NC(C)=O] | (adb) |
| 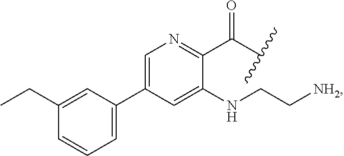 [SMILES: *C(=O)c1ncc(-c2cccc(CC)c2)cc1NCCN] | (adc) |
| 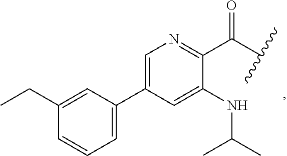 [SMILES: *C(=O)c1ncc(-c2cccc(CC)c2)cc1NC(C)C] | (add) |
| 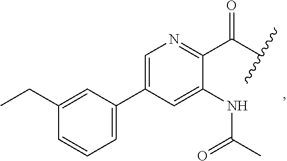 [SMILES: *C(=O)c1ncc(-c2cccc(CC)c2)cc1NC(C)=O] | (ade) |
| 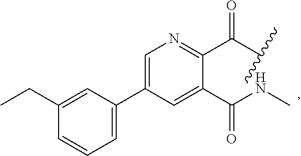 [SMILES: *C(=O)c1ncc(-c2cccc(CC)c2)cc1C(=O)NC] | (adf) |
| 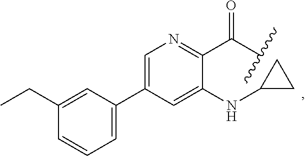 [SMILES: *C(=O)c1ncc(-c2cccc(CC)c2)cc1NC1CC1] | (adg) |
| 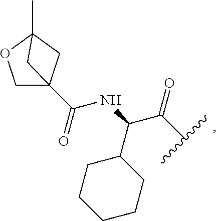 [SMILES: *C(=O)[C@H](NC(=O)C12COC(C)(C1)C2)C1CCCCC1] | (adh) |
| 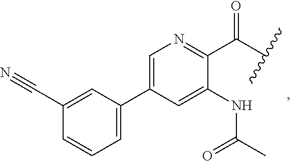 [SMILES: *C(=O)c1ncc(-c2cccc(C#N)c2)cc1NC(C)=O] | (adi) |
| 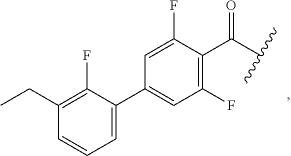 [SMILES: *C(=O)c1c(F)cc(-c2cccc(CC)c2F)cc1F] | (adj) |
| 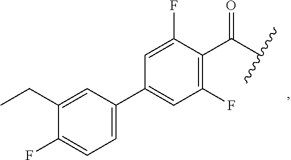 [SMILES: *C(=O)c1c(F)cc(-c2ccc(F)c(CC)c2)cc1F] | (adk) |
| 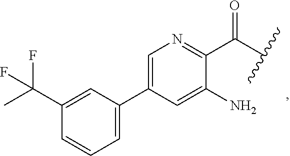 [SMILES: *C(=O)c1ncc(-c2cccc(C(C)(F)F)c2)cc1N] | (adl) |
| 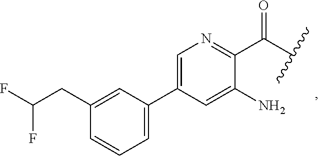 [SMILES: *C(=O)c1ncc(-c2cccc(CC(F)F)c2)cc1N] | (adm) |
| 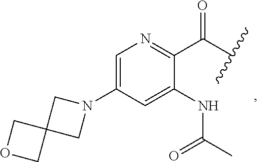 [SMILES: *C(=O)c1ncc(N2CC3(COC3)C2)cc1NC(C)=O] | (adn) |
| 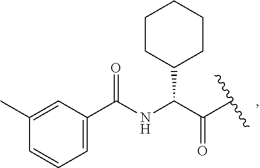 [SMILES: *C(=O)[C@H](NC(=O)c1cccc(C)c1)C1CCCCC1] | (ado) |
| 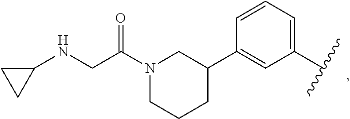 [SMILES: *c1cccc(C2CCCN(C(=O)CNC3CC3)C2)c1] | (adp) |
| 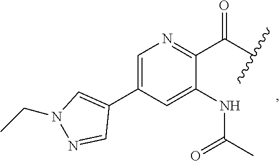 [SMILES: *C(=O)c1ncc(-c2cnn(CC)c2)cc1NC(C)=O] | (adq) |
| 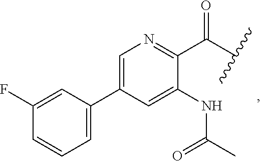 [SMILES: *C(=O)c1ncc(-c2cccc(F)c2)cc1NC(C)=O] | (adr) |
| 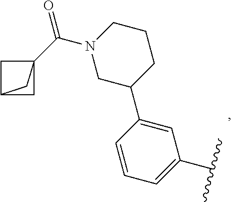 [SMILES: *c1cccc(C2CCCN(C(=O)C34CC(C3)C4)C2)c1] | (ads) |
| 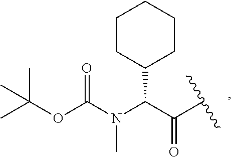 [SMILES: *C(=O)[C@@H](C1CCCCC1)N(C)C(=O)OC(C)(C)C] | (adt) |
| 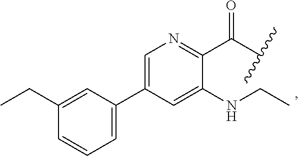 [SMILES: *C(=O)c1ncc(-c2cccc(CC)c2)cc1NCC] | (adu) |
| 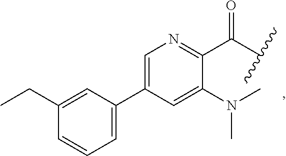 [SMILES: *C(=O)c1ncc(-c2cccc(CC)c2)cc1N(C)C] | (adv) |
| 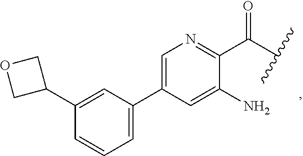 [SMILES: *C(=O)c1ncc(-c2cccc(C3COC3)c2)cc1N] | (adw) |
| 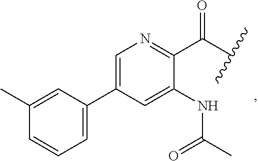 [SMILES: *C(=O)c1ncc(-c2cccc(C)c2)cc1NC(C)=O] | (adx) |
| 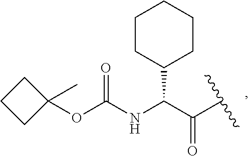 [SMILES: *C(=O)[C@H](NC(=O)OC1(C)CCC1)C1CCCCC1] | (ady) |
| 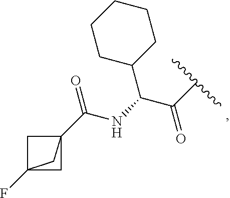 [SMILES: *C(=O)[C@H](NC(=O)C12CC(F)(C1)C2)C1CCCCC1] | (adz) |
| 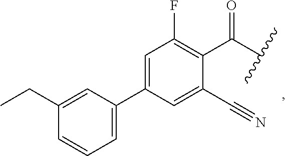 [SMILES: *C(=O)c1c(F)cc(-c2cccc(CC)c2)cc1C#N] | (aea) |
| 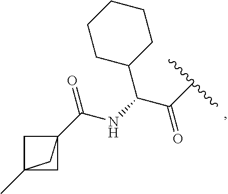 [SMILES: *C(=O)[C@H](NC(=O)C12CC(C)(C1)C2)C1CCCCC1] | (aeb) |
| 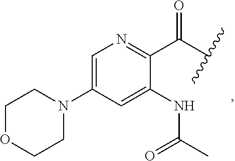 [SMILES: *C(=O)c1ncc(N2CCOCC2)cc1NC(C)=O] | (aec) |
| 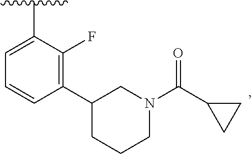 [SMILES: *c1cccc(C2CCCN(C(=O)C3CC3)C2)c1F] | (aed) |
| 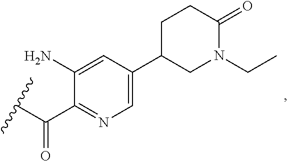 [SMILES: *C(=O)c1ncc(C2CCC(=O)N(CC)C2)cc1N] | (aee) |
| 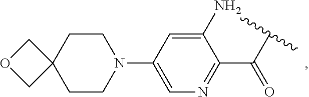 [SMILES: *C(=O)c1ncc(N2CCC3(CC2)COC3)cc1N] | (aef) |
| 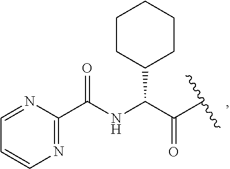 [SMILES: *C(=O)[C@H](NC(=O)c1ncccn1)C1CCCCC1] | (aeg) |
| 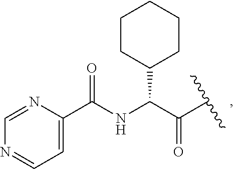 [SMILES: *C(=O)[C@H](NC(=O)c1ccncn1)C1CCCCC1] | (aeh) |
| 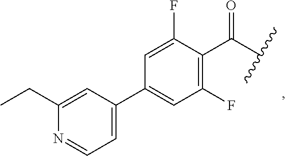 [SMILES: *C(=O)c1c(F)cc(-c2ccnc(CC)c2)cc1F] | (aei) |
| 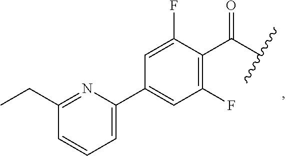 [SMILES: *C(=O)c1c(F)cc(-c2cccc(CC)n2)cc1F] | (aej) |
| 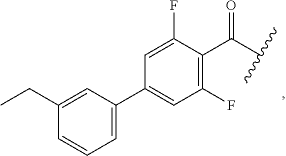 [SMILES: *C(=O)c1c(F)cc(-c2cccc(CC)c2)cc1F] | (aek) |
| 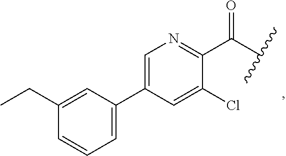 [SMILES: *C(=O)c1ncc(-c2cccc(CC)c2)cc1Cl] | (ael) |
| 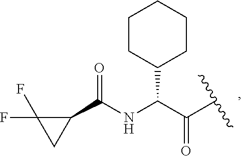 [SMILES: *C(=O)[C@H](NC(=O)[C@H]1CC1(F)F)C1CCCCC1] | (aem) |
| 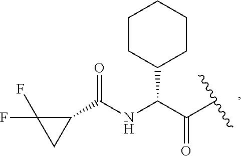 [SMILES: *C(=O)[C@H](NC(=O)[C@@H]1CC1(F)F)C1CCCCC1] | (aen) |
| 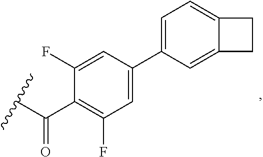 [SMILES: *C(=O)c1c(F)cc(-c2ccc3c(c2)CC3)cc1F] | (aeo) |
| 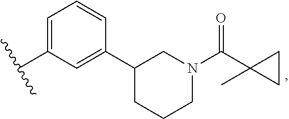 [SMILES: *c1cccc(C2CCCN(C(=O)C3(C)CC3)C2)c1] | (aep) |
| 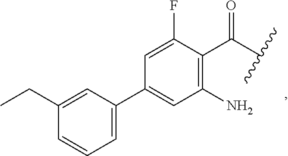 [SMILES: *C(=O)c1c(N)cc(-c2cccc(CC)c2)cc1F] | (aeq) |
| 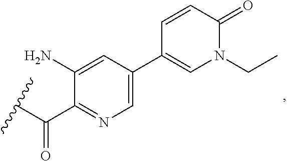 [SMILES: *C(=O)c1ncc(-c2ccc(=O)n(CC)c2)cc1N] | (aer) |
| 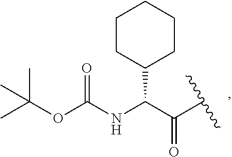 [SMILES: *C(=O)[C@H](NC(=O)OC(C)(C)C)C1CCCCC1] | (aes) |
| 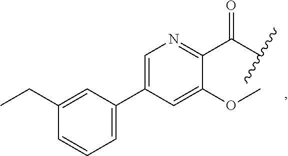 [SMILES: *C(=O)c1ncc(-c2cccc(CC)c2)cc1OC] | (aet) |
| 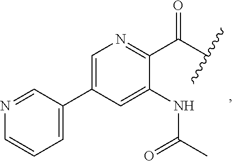 [SMILES: *C(=O)c1ncc(-c2cccnc2)cc1NC(C)=O] | (aeu) |
| 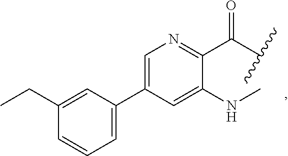 [SMILES: *C(=O)c1ncc(-c2cccc(CC)c2)cc1NC] | (aev) |
| 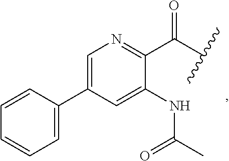 [SMILES: *C(=O)c1ncc(-c2ccccc2)cc1NC(C)=O] | (aew) |
| 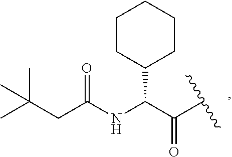 [SMILES: *C(=O)[C@H](NC(=O)CC(C)(C)C)C1CCCCC1] | (aex) |
| 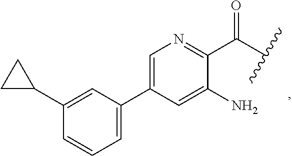 [SMILES: *C(=O)c1ncc(-c2cccc(C3CC3)c2)cc1N] | (aey) |
| 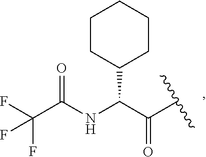 [SMILES: *C(=O)[C@H](NC(=O)C(F)(F)F)C1CCCCC1] | (aez) |
| 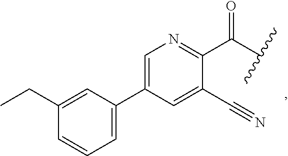 [SMILES: *C(=O)c1ncc(-c2cccc(CC)c2)cc1C#N] | (afa) |
| 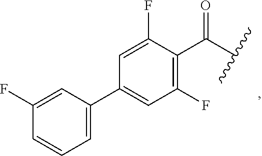 [SMILES: *C(=O)c1c(F)cc(-c2cccc(F)c2)cc1F] | (afb) |
| 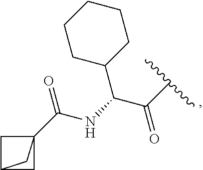 [SMILES: *C(=O)[C@H](NC(=O)C12CC(C1)C2)C1CCCCC1] | (afc) |
| 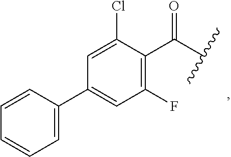 [SMILES: *C(=O)c1c(F)cc(-c2ccccc2)cc1Cl] | (afd) |
| 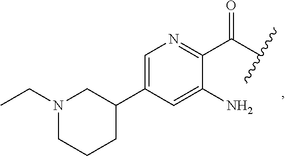 [SMILES: *C(=O)c1ncc(C2CCCN(CC)C2)cc1N] | (afe) |
| 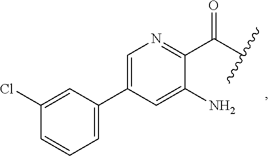 [SMILES: *C(=O)c1ncc(-c2cccc(Cl)c2)cc1N] | (aff) |
| 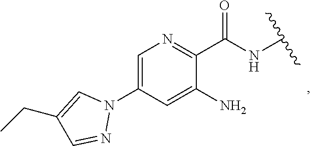 [SMILES: *NC(=O)c1ncc(-n2cc(CC)cn2)cc1N] | (afg) |
| 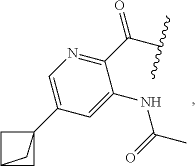 [SMILES: *C(=O)c1ncc(C23CC(C2)C3)cc1NC(C)=O] | (afh) |
| 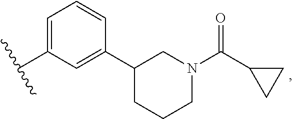 [SMILES: *c1cccc(C2CCCN(C(=O)C3CC3)C2)c1] | (afi) |
| 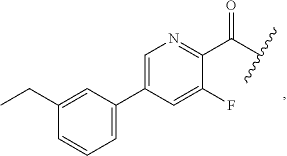 [SMILES: *C(=O)c1ncc(-c2cccc(CC)c2)cc1F] | (afj) |
| 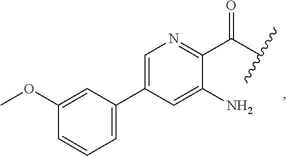 [SMILES: *C(=O)c1ncc(-c2cccc(OC)c2)cc1N] | (afk) |
| 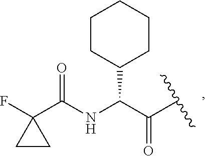 [SMILES: *C(=O)[C@H](NC(=O)C1(F)CC1)C1CCCCC1] | (afl) |
| 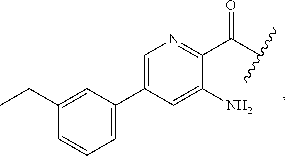 [SMILES: *C(=O)c1ncc(-c2cccc(CC)c2)cc1N] | (afm) |
| 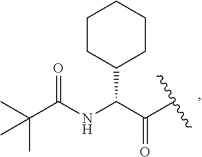 [SMILES: *C(=O)[C@H](NC(=O)C(C)(C)C)C1CCCCC1] | (afn) |
| 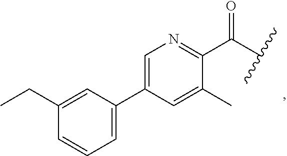 [SMILES: *C(=O)c1ncc(-c2cccc(CC)c2)cc1C] | (afo) |
| 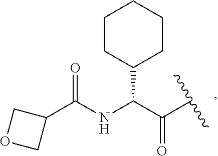 [SMILES: *C(=O)[C@H](NC(=O)C1COC1)C1CCCCC1] | (afp) |
| 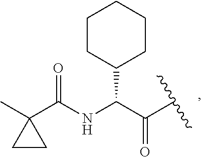 [SMILES: *C(=O)[C@H](NC(=O)C1(C)CC1)C1CCCCC1] | (afq) |
| 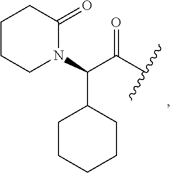 [SMILES: *C(=O)[C@@H](C1CCCCC1)N1CCCCC1=O] | (afr) |
| 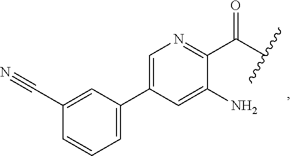 [SMILES: *C(=O)c1ncc(-c2cccc(C#N)c2)cc1N] | (afs) |
| 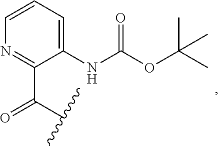 [SMILES: *C(=O)c1ncccc1NC(=O)OC(C)(C)C] | (aft) |
| 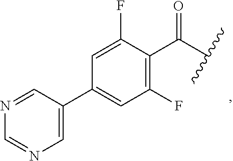 [SMILES: *C(=O)c1c(F)cc(-c2cncnc2)cc1F] | (afu) |
| 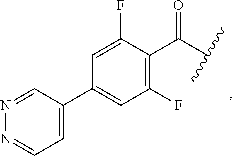 [SMILES: *C(=O)c1c(F)cc(-c2ccnnc2)cc1F] | (afv) |
| 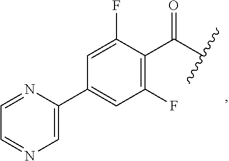 [SMILES: *C(=O)c1c(F)cc(-c2cnccn2)cc1F] | (afw) |
| 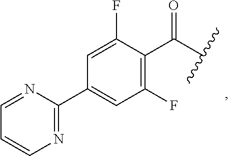 [SMILES: *C(=O)c1c(F)cc(-c2ncccn2)cc1F] | (afx) |
| 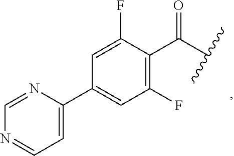 [SMILES: *C(=O)c1c(F)cc(-c2ccncn2)cc1F] | (afy) |
| 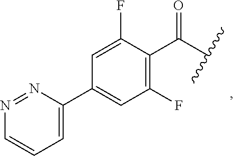 [SMILES: *C(=O)c1c(F)cc(-c2cccnn2)cc1F] | (afz) |
| 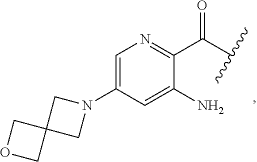 [SMILES: *C(=O)c1ncc(N2CC3(COC3)C2)cc1N] | (aga) |
| 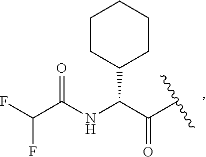 [SMILES: *C(=O)[C@H](NC(=O)C(F)F)C1CCCCC1] | (agb) |
| 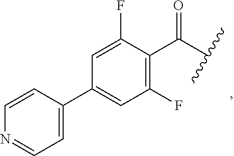 [SMILES: *C(=O)c1c(F)cc(-c2ccncc2)cc1F] | (agc) |
| 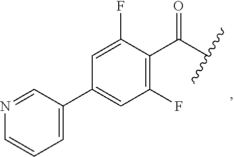 [SMILES: *C(=O)c1c(F)cc(-c2cccnc2)cc1F] | (agd) |
| 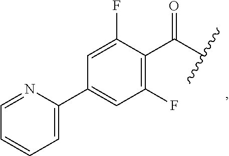 [SMILES: *C(=O)c1c(F)cc(-c2ccccn2)cc1F] | (age) |
| 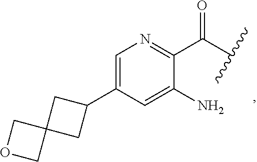 [SMILES: *C(=O)c1ncc(C2CC3(COC3)C2)cc1N] | (agf) |
| 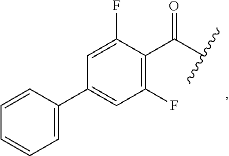 [SMILES: *C(=O)c1c(F)cc(-c2ccccc2)cc1F] | (agg) |
| 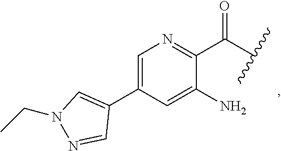 [SMILES: *C(=O)c1ncc(-c2cnn(CC)c2)cc1N] | (agh) |
| 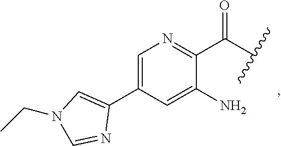 [SMILES: *C(=O)c1ncc(-c2cn(CC)cn2)cc1N] | (agi) |
| 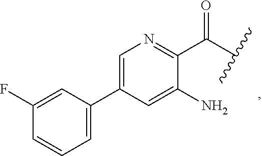 [SMILES: *C(=O)c1ncc(-c2cccc(F)c2)cc1N] | (agj) |
| 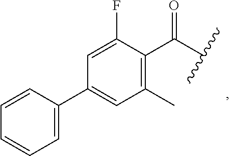 [SMILES: *C(=O)c1c(C)cc(-c2ccccc2)cc1F] | (agk) |
| 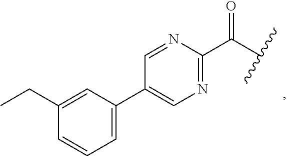 [SMILES: *C(=O)c1ncc(-c2cccc(CC)c2)cn1] | (agl) |
| 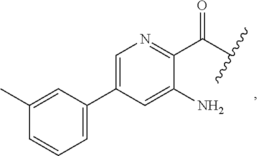 [SMILES: *C(=O)c1ncc(-c2cccc(C)c2)cc1N] | (agm) |
| 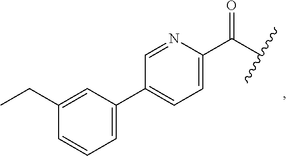 [SMILES: *C(=O)c1ccc(-c2cccc(CC)c2)cn1] | (agn) |
| 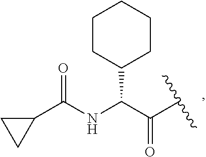 [SMILES: *C(=O)[C@H](NC(=O)C1CC1)C1CCCCC1] | (ago) |
| 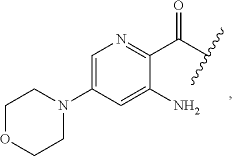 [SMILES: *C(=O)c1ncc(N2CCOCC2)cc1N] | (agp) |
| 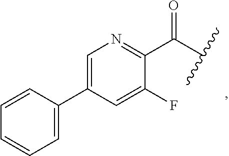 [SMILES: *C(=O)c1ncc(-c2ccccc2)cc1F] | (agq) |
| 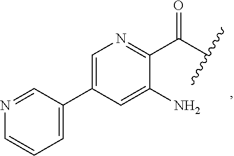 [SMILES: *C(=O)c1ncc(-c2cccnc2)cc1N] | (agr) |
| 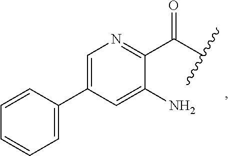 [SMILES: *C(=O)c1ncc(-c2ccccc2)cc1N] | (ags) |
| 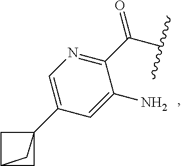 [SMILES: *C(=O)c1ncc(C23CC(C2)C3)cc1N] | (agt) |
| 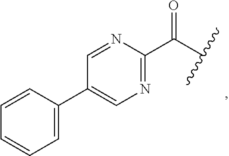 [SMILES: *C(=O)c1ncc(-c2ccccc2)cn1] | (agu) |
| 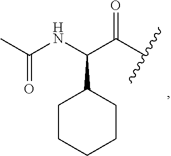 [SMILES: *C(=O)[C@H](NC(C)=O)C1CCCCC1] | (agv) |
| 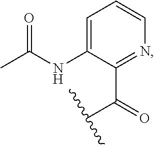 [SMILES: *C(=O)c1ncccc1NC(C)=O] | (agw) |
| 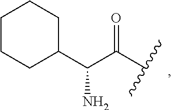 [SMILES: *C(=O)[C@H](N)C1CCCCC1] | (agx) |
| 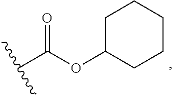 [SMILES: *C(=O)OC1CCCCC1] | (agy) |
| 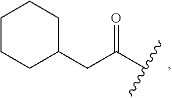 [SMILES: *C(=O)CC1CCCCC1] | (agz) |
| 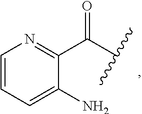 [SMILES: *C(=O)c1ncccc1N] | (aha) |
| 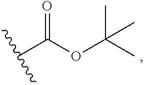 [SMILES: *C(=O)OC(C)(C)C] | (ahb) |
| 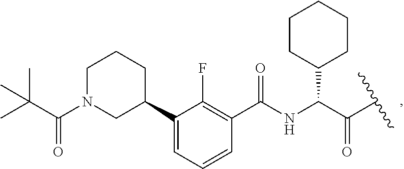 [SMILES: *C(=O)[C@H](NC(=O)c1cccc([C@@H]2CCCN(C(=O)C(C)(C)C)C2)c1F)C1CCCCC1] | (ahc) |
| 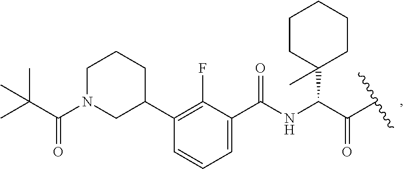 [SMILES: *C(=O)[C@H](NC(=O)c1cccc(C2CCCN(C(=O)C(C)(C)C)C2)c1F)C1(C)CCCCC1] | (ahd) |
| 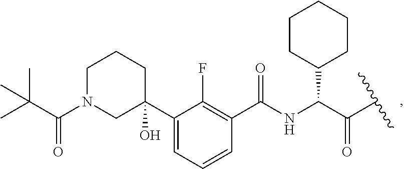 [SMILES: *C(=O)[C@H](NC(=O)c1cccc([C@]2(O)CCCN(C(=O)C(C)(C)C)C2)c1F)C1CCCCC1] | (ahe) |
| 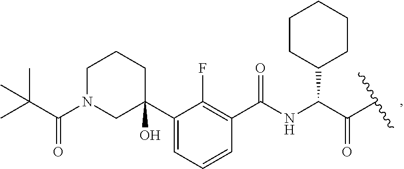 [SMILES: *C(=O)[C@H](NC(=O)c1cccc([C@@]2(O)CCCN(C(=O)C(C)(C)C)C2)c1F)C1CCCCC1] | (ahf) |
| 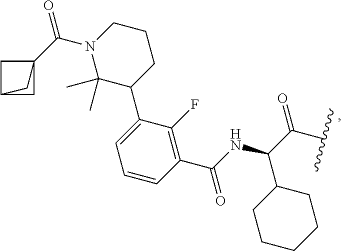 [SMILES: *C(=O)[C@H](NC(=O)c1cccc(C2CCCN(C(=O)C34CC(C3)C4)C2(C)C)c1F)C1CCCCC1] | (ahg) |
| 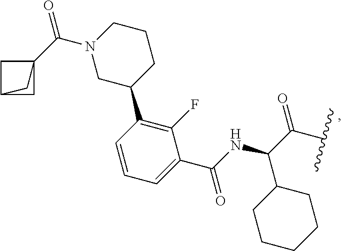 [SMILES: *C(=O)[C@H](NC(=O)c1cccc([C@@H]2CCCN(C(=O)C34CC(C3)C4)C2)c1F)C1CCCCC1] | (ahh) |
| 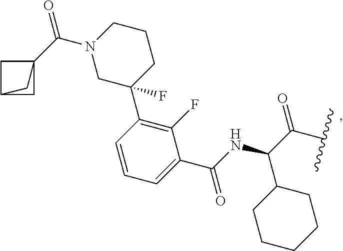 [SMILES: *C(=O)[C@H](NC(=O)c1cccc([C@]2(F)CCCN(C(=O)C34CC(C3)C4)C2)c1F)C1CCCCC1] | (ahi) |
| 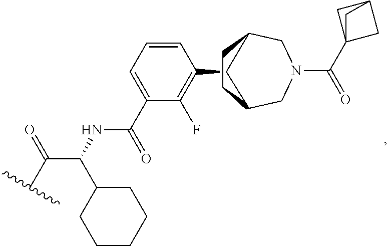 | (ahj) |
| 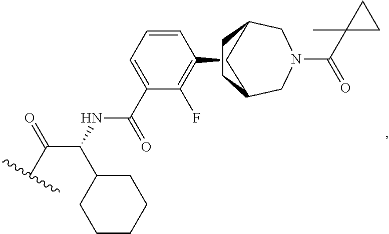 | (ahk) |
| 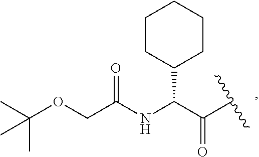 [SMILES: *C(=O)[C@H](NC(=O)COC(C)(C)C)C1CCCCC1] | (ahl) |
| 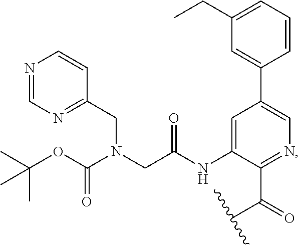 [SMILES: *C(=O)c1ncc(-c2cccc(CC)c2)cc1NC(=O)CN(Cc1ccncn1)C(=O)OC(C)(C)C] | (ahm) |
| 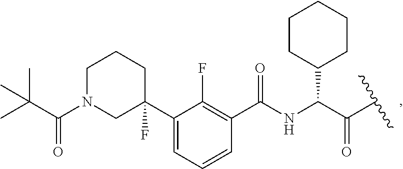 [SMILES: *C(=O)[C@H](NC(=O)c1cccc([C@]2(F)CCCN(C(=O)C(C)(C)C)C2)c1F)C1CCCCC1] | (ahn) |
| 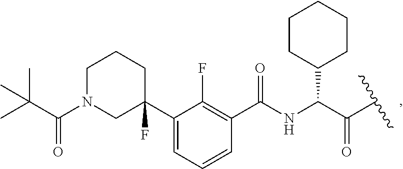 [SMILES: *C(=O)[C@H](NC(=O)c1cccc([C@@]2(F)CCCN(C(=O)C(C)(C)C)C2)c1F)C1CCCCC1] | (aho) |
| 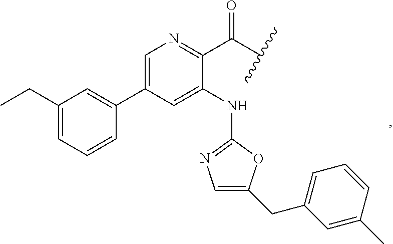 [SMILES: *C(=O)c1ncc(-c2cccc(CC)c2)cc1Nc1ncc(Cc2cccc(C)c2)o1] | (ahp) |
| 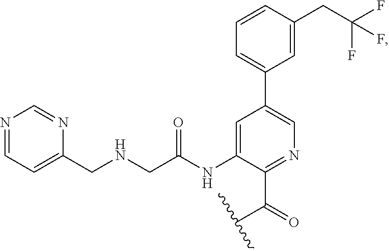 [SMILES: *C(=O)c1ncc(-c2cccc(CC(F)(F)F)c2)cc1NC(=O)CNCc1ccncn1] | (ahq) |
| 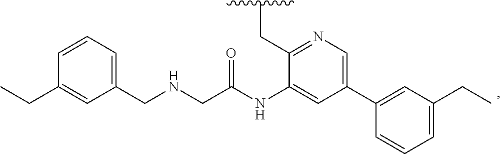 [SMILES: *Cc1ncc(-c2cccc(CC)c2)cc1NC(=O)CNCc1cccc(CC)c1] | (ahr) |
| 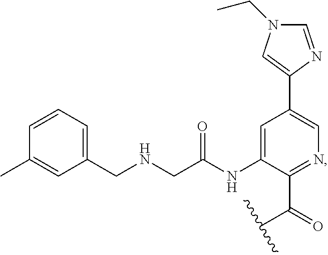 [SMILES: *C(=O)c1ncc(-c2cn(CC)cn2)cc1NC(=O)CNCc1cccc(C)c1] | (ahs) |
| 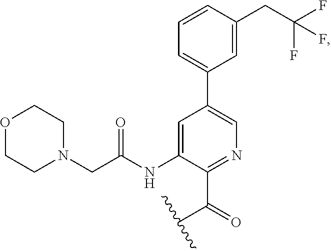 [SMILES: *C(=O)c1ncc(-c2cccc(CC(F)(F)F)c2)cc1NC(=O)CN1CCOCC1] | (aht) |
| 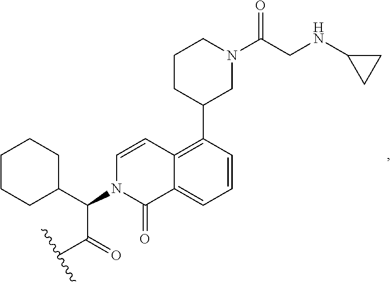 [SMILES: *C(=O)[C@@H](C1CCCCC1)n1ccc2c(C3CCCN(C(=O)CNC4CC4)C3)cccc2c1=O] | (ahu) |
| 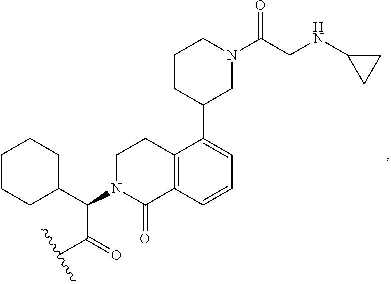 [SMILES: *C(=O)[C@@H](C1CCCCC1)N1CCc2c(cccc2C2CCCN(C(=O)CNC3CC3)C2)C1=O] | (ahv) |
| 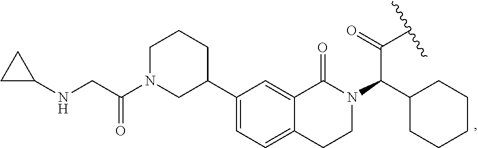 [SMILES: *C(=O)[C@@H](C1CCCCC1)N1CCc2ccc(C3CCCN(C(=O)CNC4CC4)C3)cc2C1=O] | (ahw) |
| 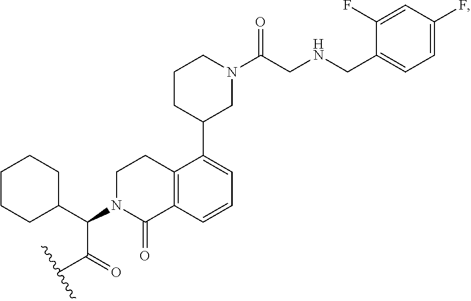 [SMILES: *C(=O)[C@@H](C1CCCCC1)N1CCc2c(cccc2C2CCCN(C(=O)CNCc3ccc(F)cc3F)C2)C1=O] | (ahx) |
| 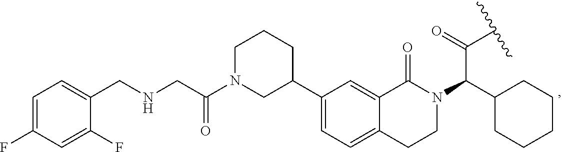 [SMILES: *C(=O)[C@@H](C1CCCCC1)N1CCc2ccc(C3CCCN(C(=O)CNCc4ccc(F)cc4F)C3)cc2C1=O] | (ahy) |
| 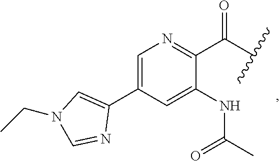 [SMILES: *C(=O)c1ncc(-c2cn(CC)cn2)cc1NC(C)=O] | (ahz) |
| 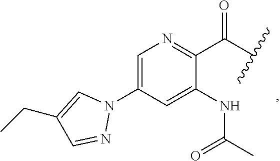 [SMILES: *C(=O)c1ncc(-n2cc(CC)cn2)cc1NC(C)=O] | (aia) |
| 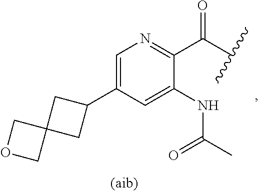 [SMILES: *C(=O)c1ncc(C2CC3(COC3)C2)cc1NC(C)=O] | (aib) |
| 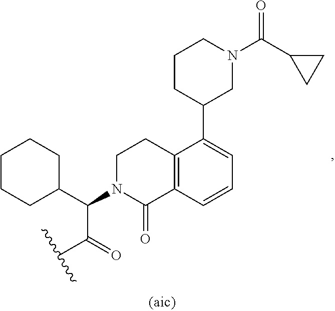 [SMILES: *C(=O)[C@@H](C1CCCCC1)N1CCc2c(cccc2C2CCCN(C(=O)C3CC3)C2)C1=O] | (aic) |
| 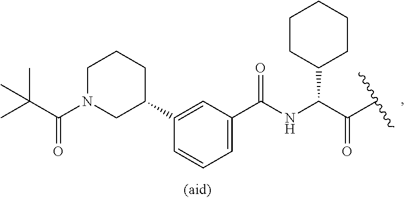 [SMILES: *C(=O)[C@H](NC(=O)c1cccc([C@H]2CCCN(C(=O)C(C)(C)C)C2)c1)C1CCCCC1] | (aid) |
| 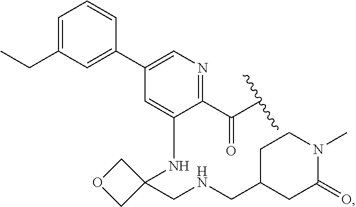 [SMILES: *C(=O)c1ncc(-c2cccc(CC)c2)cc1NC1(CNCC2CCN(C)C(=O)C2)COC1] | (aie) |
| 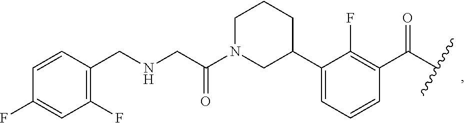 [SMILES: *C(=O)c1cccc(C2CCCN(C(=O)CNCc3ccc(F)cc3F)C2)c1F] | (aif) |
| 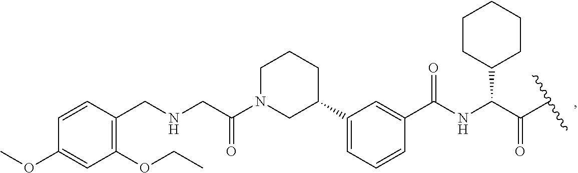 [SMILES: *C(=O)[C@H](NC(=O)c1cccc([C@H]2CCCN(C(=O)CNCc3ccc(OC)cc3OCC)C2)c1)C1CCCCC1] | (aig) |
| 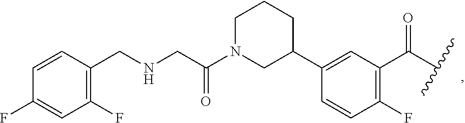 [SMILES: *C(=O)c1cc(C2CCCN(C(=O)CNCc3ccc(F)cc3F)C2)ccc1F] | (aih) |
| 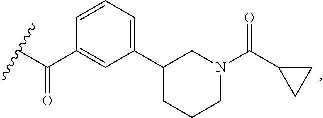 [SMILES: *C(=O)c1cccc(C2CCCN(C(=O)C3CC3)C2)c1] | (aij) |
| 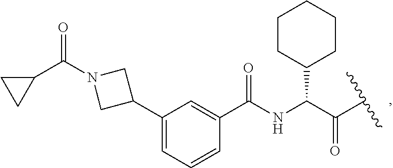 [SMILES: *C(=O)[C@H](NC(=O)c1cccc(C2CN(C(=O)C3CC3)C2)c1)C1CCCCC1] | (aik) |
| 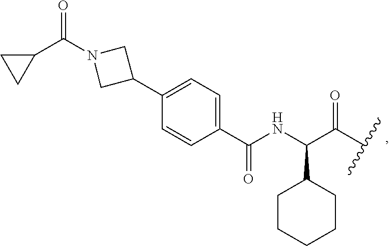 [SMILES: *C(=O)[C@H](NC(=O)c1ccc(C2CN(C(=O)C3CC3)C2)cc1)C1CCCCC1] | (ail) |
| 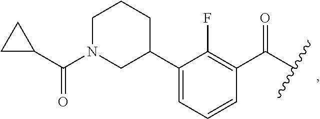 [SMILES: *C(=O)c1cccc(C2CCCN(C(=O)C3CC3)C2)c1F] | (aim) |
| 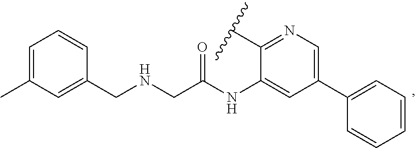 [SMILES: *c1ncc(-c2ccccc2)cc1NC(=O)CNCc1cccc(C)c1] | (ain) |
| 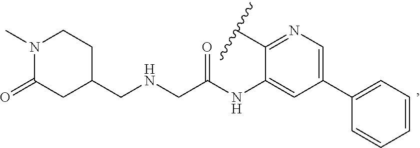 [SMILES: *c1ncc(-c2ccccc2)cc1NC(=O)CNCC1CCN(C)C(=O)C1] | (aio) |
| 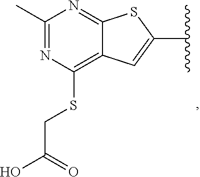 [SMILES: *c1cc2c(SCC(=O)O)nc(C)nc2s1] | (aip) |
| 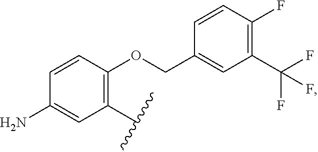 [SMILES: *c1cc(N)ccc1OCc1ccc(F)c(C(F)(F)F)c1] | (aiq) |
| 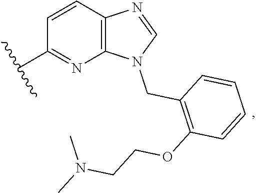 [SMILES: *c1ccc2ncn(Cc3ccccc3OCCN(C)C)c2n1] | (air) |
| 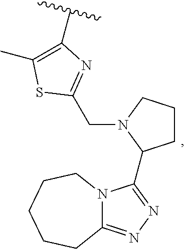 [SMILES: *c1nc(CN2CCCC2c2nnc3n2CCCCC3)sc1C] | (ais) |
| 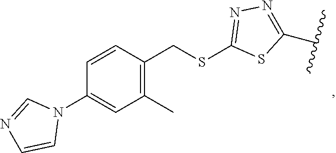 [SMILES: *c1nnc(SCc2ccc(-n3ccnc3)cc2C)s1] | (ait) |
| 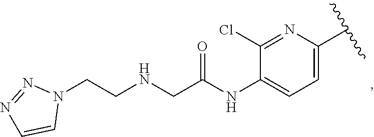 [SMILES: *c1ccc(NC(=O)CNCCn2ccnn2)c(Cl)n1] | (aiu) |
| 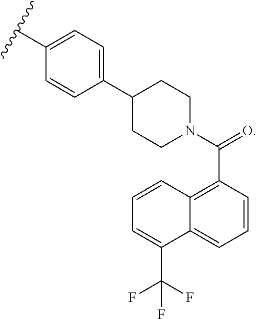 [SMILES: *c1ccc(C2CCN(C(=O)c3cccc4c(C(F)(F)F)cccc34)CC2)cc1] | (aiv) |

TABLE B
Exemplified Linkers (L)

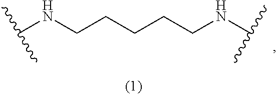 [SMILES: *NCCCCCN*]

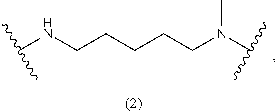 [SMILES: *NCCCCCN(*)C]

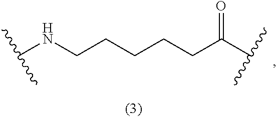 [SMILES: *NCCCCCC(*)=O]

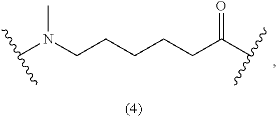 [SMILES: *C(=O)CCCCCN(*)C]

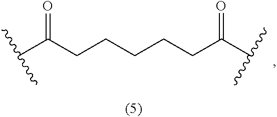 [SMILES: *C(=O)CCCCCC(*)=O]

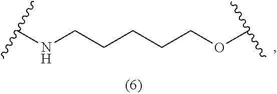 [SMILES: *NCCCCCO*]

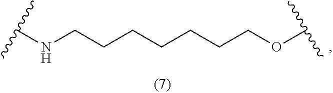 [SMILES: *NCCCCCCCO*]

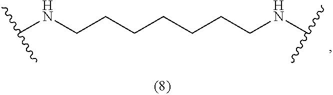 [SMILES: *NCCCCCCCN*]

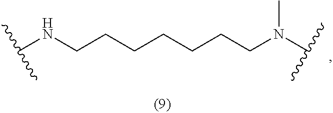 [SMILES: *NCCCCCCCN(*)C]

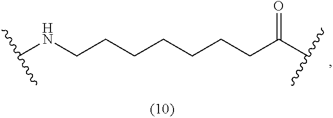 [SMILES: *NCCCCCCCC(*)=O]

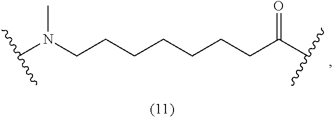 [SMILES: *C(=O)CCCCCCCN(*)C]

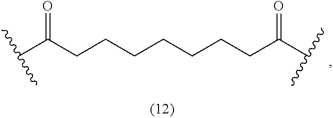 [SMILES: *C(=O)CCCCCCCC(*)=O]

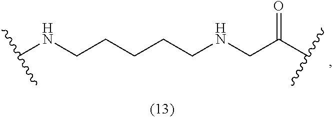 [SMILES: *NCCCCCNCC(*)=O]

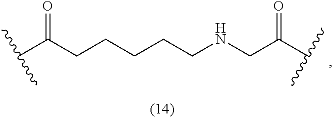 [SMILES: *C(=O)CCCCCNCC(*)=O]

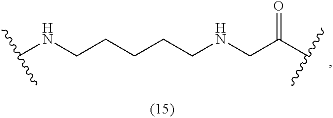 [SMILES: *NCCCCCNCC(*)=O]

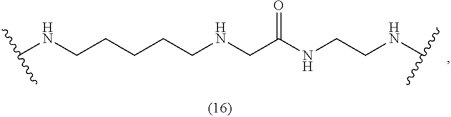 [SMILES: *NCCCCCNCC(=O)NCCN*]

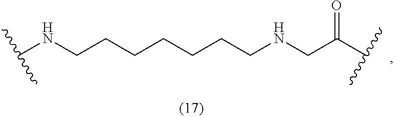 [SMILES: *NCCCCCCCNCC(*)=O]

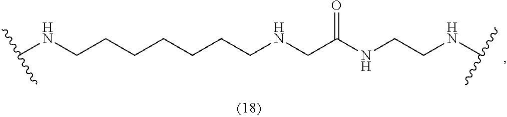 [SMILES: *NCCCCCCCNCC(=O)NCCN*]

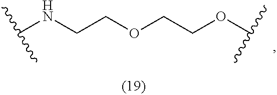 [SMILES: *NCCOCCO*]

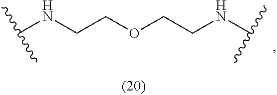 [SMILES: *NCCOCCN*]

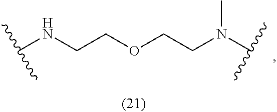 [SMILES: *NCCOCCN(*)C]

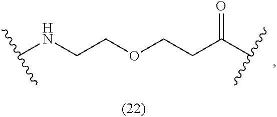 [SMILES: *NCCOCCC(*)=O]

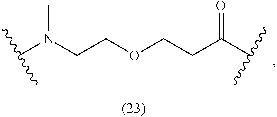 [SMILES: *C(=O)CCOCCN(*)C]

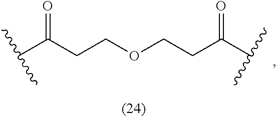 [SMILES: *C(=O)CCOCCC(*)=O]

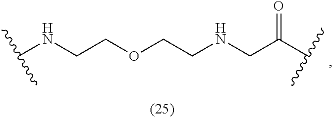 [SMILES: *NCCOCCNCC(*)=O]

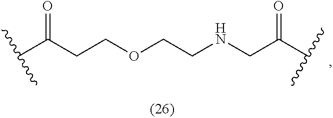 [SMILES: *C(=O)CCOCCNCC(*)=O]

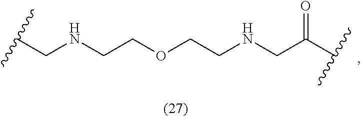 [SMILES: *CNCCOCCNCC(*)=O]

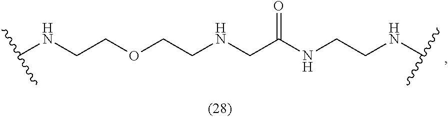 [SMILES: *NCCNC(=O)CNCCOCCN*]

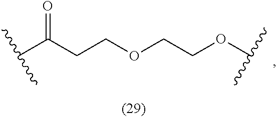 [SMILES: *OCCOCCC(*)=O]

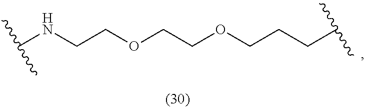 [SMILES: *CCCOCCOCCN*]

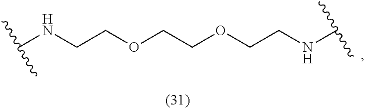 [SMILES: *NCCOCCOCCN*]

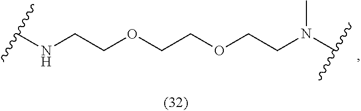 [SMILES: *NCCOCCOCCN(*)C]

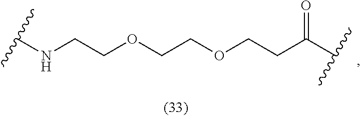 [SMILES: *NCCOCCOCCC(*)=O]

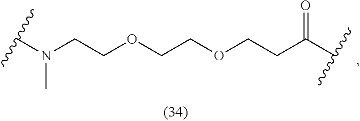 [SMILES: *C(=O)CCOCCOCCN(*)C]

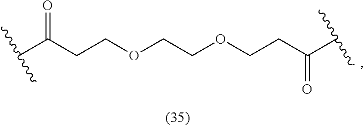 [SMILES: *C(=O)CCOCCOCCC(*)=O]

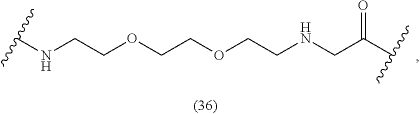 [SMILES: *NCCOCCOCCNCC(*)=O]

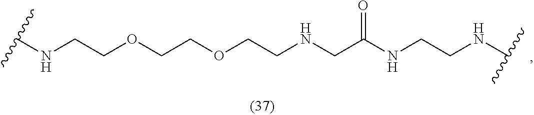 [SMILES: *NCCNC(=O)CNCCOCCOCCN*]

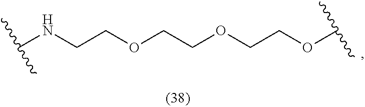 [SMILES: *NCCOCCOCCO*]

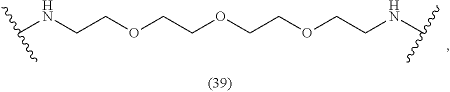 [SMILES: *NCCOCCOCCOCCN*]

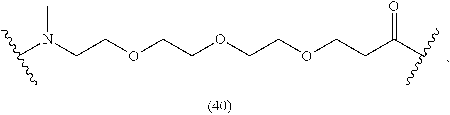 [SMILES: *C(=O)CCOCCOCCOCCN(*)C]

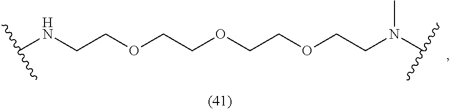 [SMILES: *NCCOCCOCCOCCN(*)C]

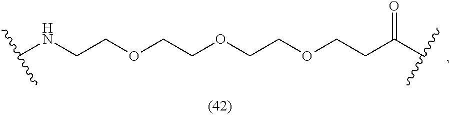 [SMILES: *NCCOCCOCCOCCC(*)=O]

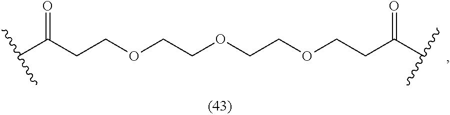 [SMILES: *C(=O)CCOCCOCCOCCC(*)=O]

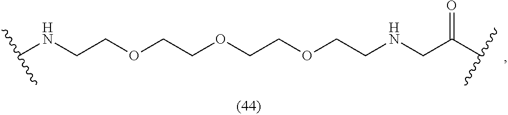 [SMILES: *NCCOCCOCCOCCNCC(*)=O]

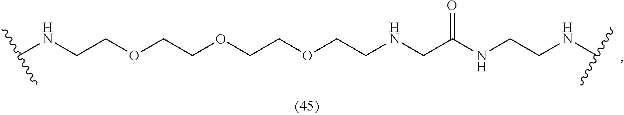 [SMILES: *NCCNC(=O)CNCCOCCOCCOCCN*]

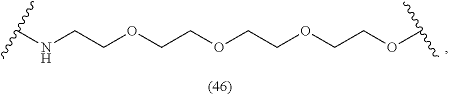 [SMILES: *NCCOCCOCCOCCO*]

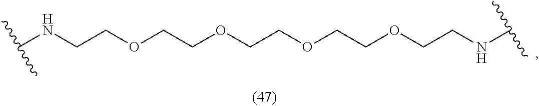 [SMILES: *NCCOCCOCCOCCOCCN*]

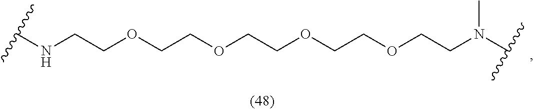 [SMILES: *NCCOCCOCCOCCOCCN(*)C]

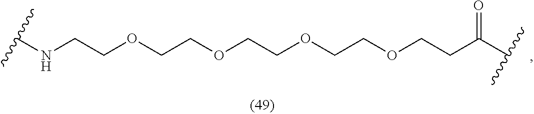 [SMILES: *NCCOCCOCCOCCOCCC(*)=O]

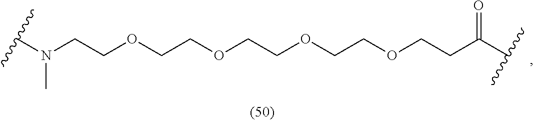 [SMILES: *C(=O)CCOCCOCCOCCOCCN(*)C]

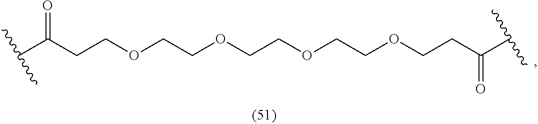 [SMILES: *C(=O)CCOCCOCCOCCOCCC(*)=O]

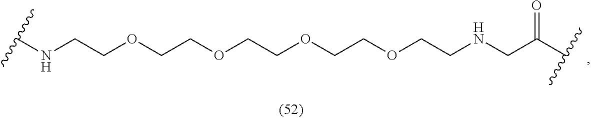 [SMILES: *NCCOCCOCCOCCOCCNCC(*)=O]

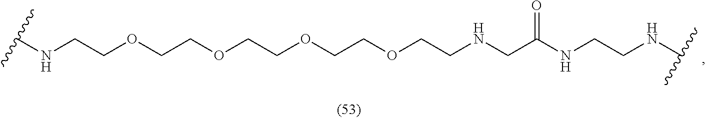 [SMILES: *NCCNC(=O)CNCCOCCOCCOCCOCCN*]

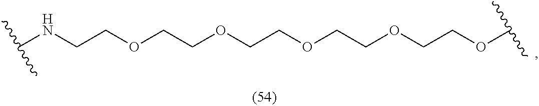 [SMILES: *NCCOCCOCCOCCOCCO*]

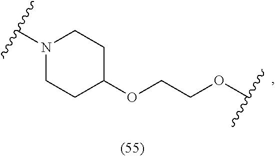 [SMILES: *OCCOC1CCN(*)CC1]

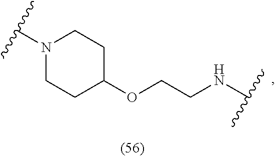 [SMILES: *NCCOC1CCN(*)CC1]

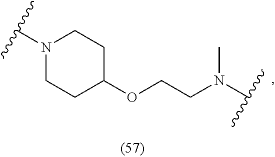 [SMILES: *N(C)CCOC1CCN(*)CC1]

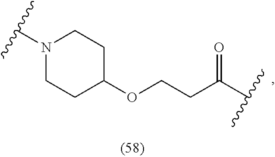 [SMILES: *C(=O)CCOC1CCN(*)CC1]

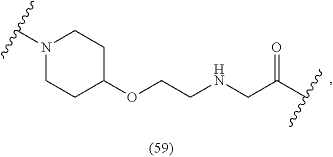 [SMILES: *C(=O)CNCCOC1CCN(*)CC1]

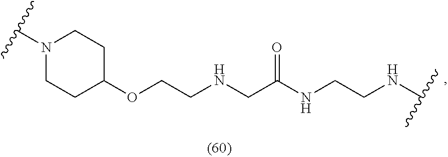 [SMILES: *NCCNC(=O)CNCCOC1CCN(*)CC1]

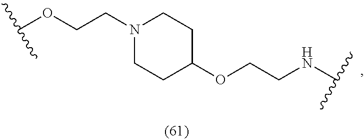 [SMILES: *NCCOC1CCN(CCO*)CC1]

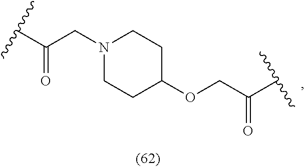 [SMILES: *C(=O)COC1CCN(CC(*)=O)CC1]

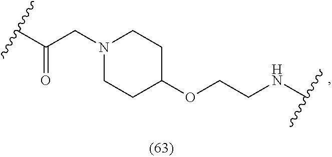 [SMILES: *NCCOC1CCN(CC(*)=O)CC1]

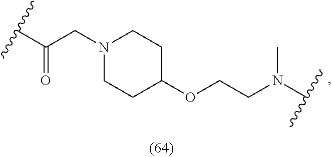 [SMILES: *C(=O)CN1CCC(OCCN(*)C)CC1]

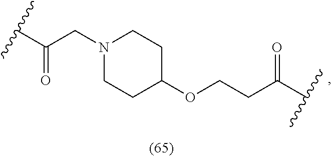 [SMILES: *C(=O)CCOC1CCN(CC(*)=O)CC1]

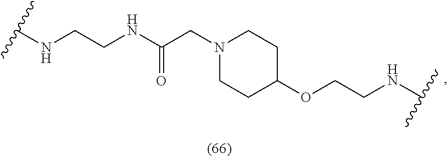 [SMILES: *NCCNC(=O)CN1CCC(OCCN*)CC1]

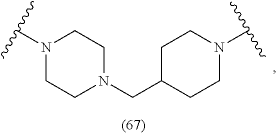 [SMILES: *N1CCC(CN2CCN(*)CC2)CC1]

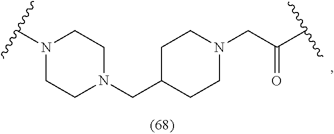 [SMILES: *C(=O)CN1CCC(CN2CCN(*)CC2)CC1]

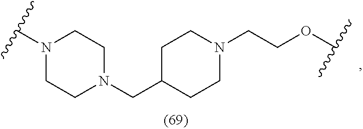 [SMILES: *OCCN1CCC(CN2CCN(*)CC2)CC1]

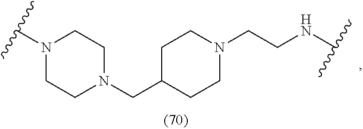 [SMILES: *NCCN1CCC(CN2CCN(*)CC2)CC1]

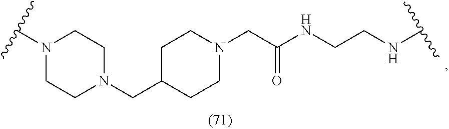 [SMILES: *NCCNC(=O)CN1CCC(CN2CCN(*)CC2)CC1]

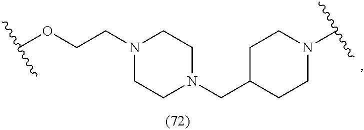 [SMILES: *OCCN1CCN(CC2CCN(*)CC2)CC1]

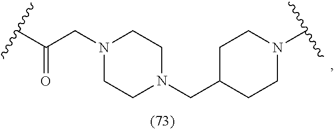 [SMILES: *C(=O)CN1CCN(CC2CCN(*)CC2)CC1]

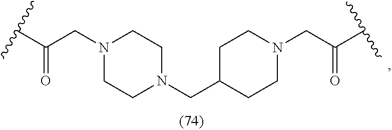 [SMILES: *C(=O)CN1CCC(CN2CCN(CC(*)=O)CC2)CC1]

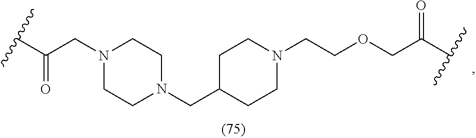 [SMILES: *C(=O)COCCN1CCC(CN2CCN(CC(*)=O)CC2)CC1]

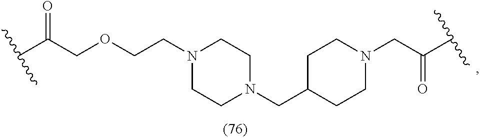 [SMILES: *C(=O)COCCN1CCN(CC2CCN(CC(*)=O)CC2)CC1]

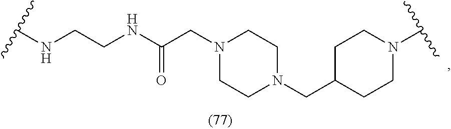 [SMILES: *NCCNC(=O)CN1CCN(CC2CCN(*)CC2)CC1]

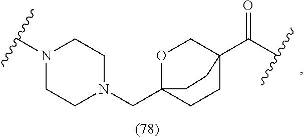 [SMILES: *C(=O)C12CCC(CN3CCN(*)CC3)(CC1)OC2]

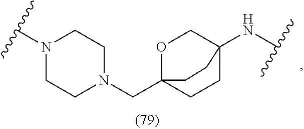 [SMILES: *NC12CCC(CN3CCN(*)CC3)(CC1)OC2]

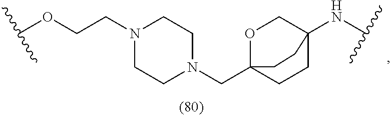 [SMILES: *NC12CCC(CN3CCN(CCO*)CC3)(CC1)OC2]

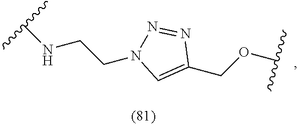 [SMILES: *NCCn1cc(CO*)nn1]

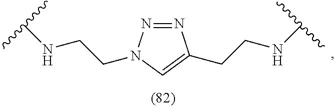 [SMILES: *NCCc1cn(CCN*)nn1]

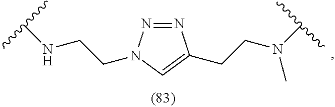 [SMILES: *NCCn1cc(CCN(*)C)nn1]

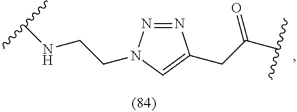 [SMILES: *NCCn1cc(CC(*)=O)nn1]

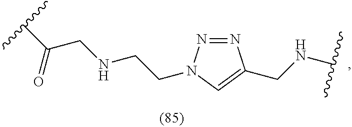 [SMILES: *NCc1cn(CCNCC(*)=O)nn1]

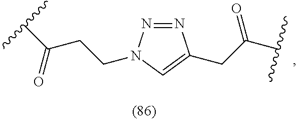 [SMILES: *C(=O)CCn1cc(CC(*)=O)nn1]

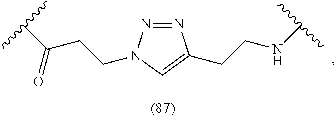 [SMILES: *NCCc1cn(CCC(*)=O)nn1]

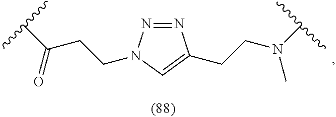 [SMILES: *C(=O)CCn1cc(CCN(*)C)nn1]

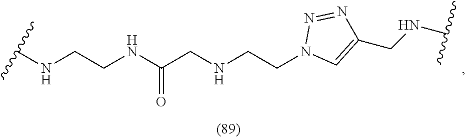 [SMILES: *NCCNC(=O)CNCCn1cc(CN*)nn1]

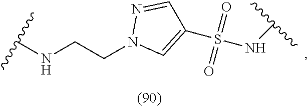 [SMILES: *NCCn1cc(S(=O)(=O)N*)cn1]

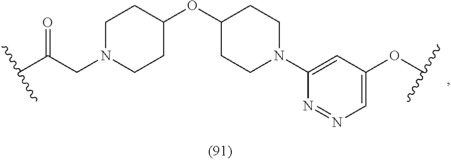 [SMILES: *Oc1cnnc(N2CCC(OC3CCN(CC(*)=O)CC3)CC2)c1]

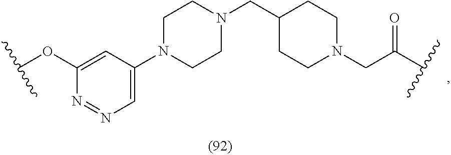 [SMILES: *Oc1cc(N2CCN(CC3CCN(CC(*)=O)CC3)CC2)cnn1]

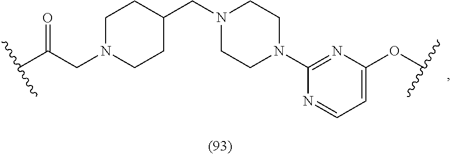 [SMILES: *Oc1ccnc(N2CCN(CC3CCN(CC(*)=O)CC3)CC2)n1]

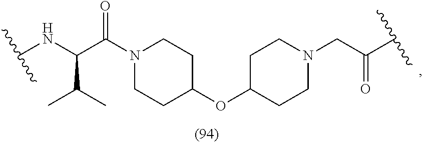 [SMILES: *N[C@@H](C(=O)N1CCC(OC2CCN(CC(*)=O)CC2)CC1)C(C)C]

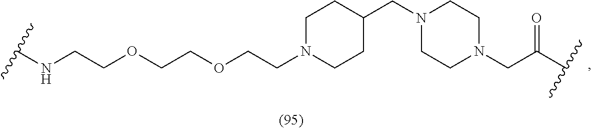 [SMILES: *NCCOCCOCCN1CCC(CN2CCN(CC(*)=O)CC2)CC1]

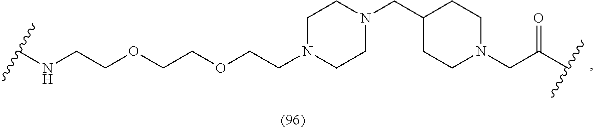 [SMILES: *NCCOCCOCCN1CCN(CC2CCN(CC(*)=O)CC2)CC1]

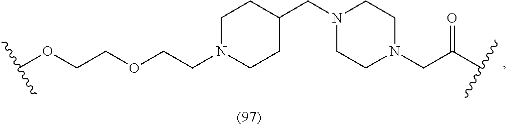 [SMILES: *OCCOCCN1CCC(CN2CCN(CC(*)=O)CC2)CC1]

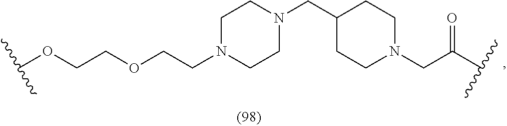 [SMILES: *OCCOCCN1CCN(CC2CCN(CC(*)=O)CC2)CC1]

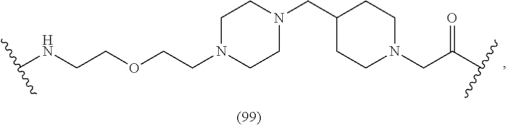 [SMILES: *NCCOCCN1CCN(CC2CCN(CC(*)=O)CC2)CC1]

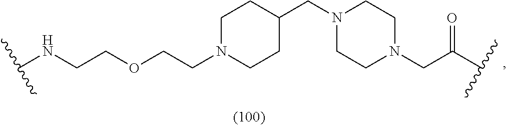 [SMILES: *NCCOCCN1CCC(CN2CCN(CC(*)=O)CC2)CC1]

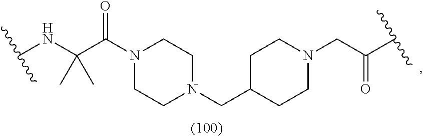 [SMILES: *NC(C)(C)C(=O)N1CCN(CC2CCN(CC(*)=O)CC2)CC1]

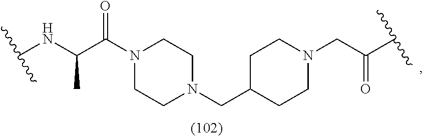 [SMILES: *N[C@H](C)C(=O)N1CCN(CC2CCN(CC(*)=O)CC2)CC1]

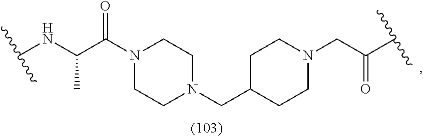 [SMILES: *N[C@@H](C)C(=O)N1CCN(CC2CCN(CC(*)=O)CC2)CC1]

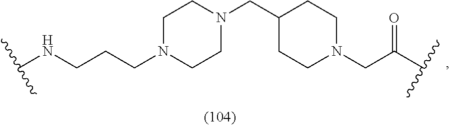 [SMILES: *NCCCN1CCN(CC2CCN(CC(*)=O)CC2)CC1]

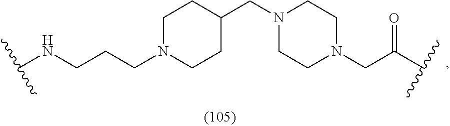 [SMILES: *NCCCN1CCC(CN2CCN(CC(*)=O)CC2)CC1]

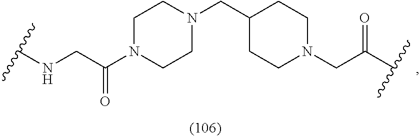 [SMILES: *NCC(=O)N1CCN(CC2CCN(CC(*)=O)CC2)CC1]

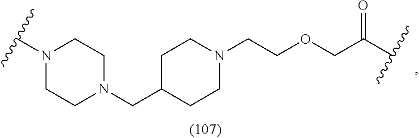 [SMILES: *C(=O)COCCN1CCC(CN2CCN(*)CC2)CC1]

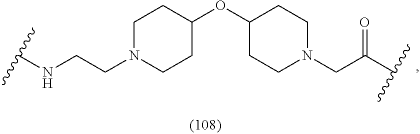 [SMILES: *NCCN1CCC(OC2CCN(CC(*)=O)CC2)CC1]

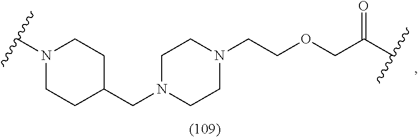 [SMILES: *C(=O)COCCN1CCN(CC2CCN(*)CC2)CC1]

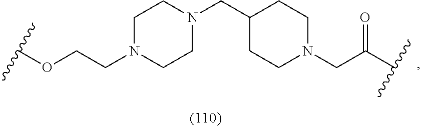 [SMILES: *OCCN1CCN(CC2CCN(CC(*)=O)CC2)CC1]

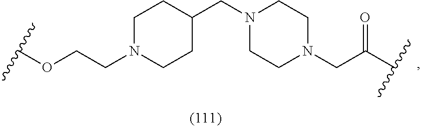 [SMILES: *OCCN1CCC(CN2CCN(CC(*)=O)CC2)CC1]

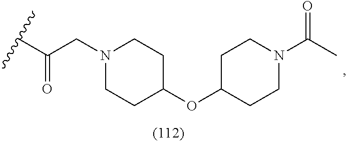 [SMILES: *C(=O)CN1CCC(OC2CCN(C(C)=O)CC2)CC1]

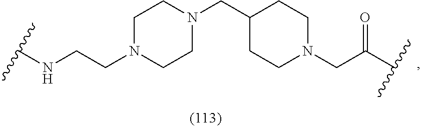 [SMILES: *NCCN1CCN(CC2CCN(CC(*)=O)CC2)CC1]

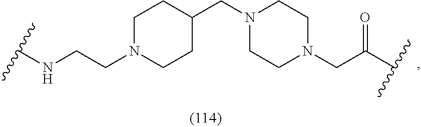 [SMILES: *NCCN1CCC(CN2CCN(CC(*)=O)CC2)CC1]

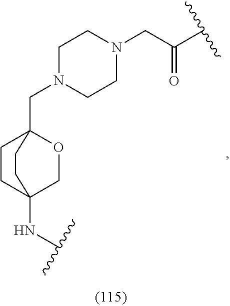 [SMILES: *NC12CCC(CN3CCN(CC(*)=O)CC3)(CC1)OC2]

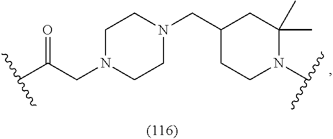 [SMILES: *C(=O)CN1CCN(CC2CCN(*)C(C)(C)C2)CC1]

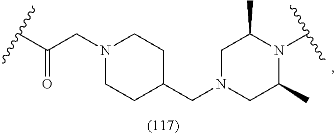 [SMILES: *C(=O)CN1CCC(CN2C[C@@H](C)N(*)[C@@H](C)C2)CC1]

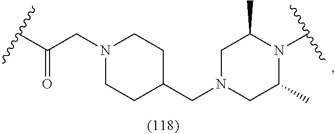 [SMILES: *C(=O)CN1CCC(CN2C[C@@H](C)N(*)[C@H](C)C2)CC1]

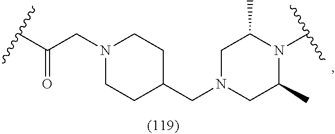 [SMILES: *C(=O)CN1CCC(CN2C[C@H](C)N(*)[C@@H](C)C2)CC1]

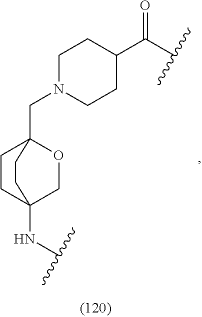 [SMILES: *NC12CCC(CN3CCC(C(*)=O)CC3)(CC1)OC2]

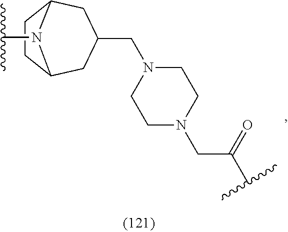 [SMILES: *C(=O)CN1CCN(CC2CC3CCC(C2)N3*)CC1]

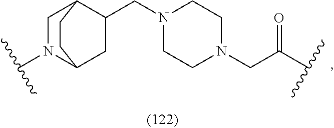 [SMILES: *C(=O)CN1CCN(CC2CC3CCC2CN3*)CC1]

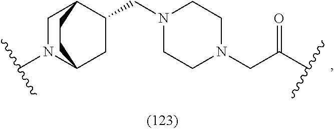 [SMILES: *C(=O)CN1CCN(C[C@@H]2C[C@H]3CC[C@@H]2CN3*)CC1]

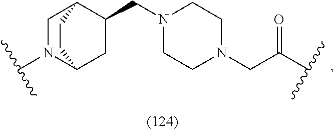 [SMILES: *C(=O)CN1CCN(C[C@H]2C[C@@H]3CC[C@H]2CN3*)CC1]

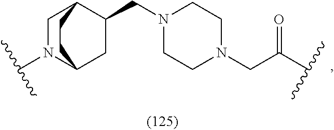 [SMILES: *C(=O)CN1CCN(C[C@H]2C[C@H]3CC[C@@H]2CN3*)CC1]

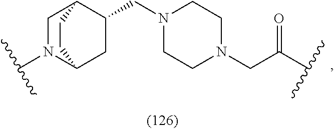 [SMILES: *C(=O)CN1CCN(C[C@@H]2C[C@@H]3CC[C@H]2CN3*)CC1]

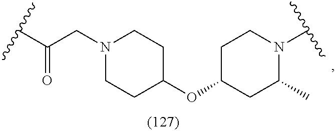 [SMILES: *C(=O)CN1CCC(O[C@@H]2CCN(*)[C@H](C)C2)CC1]

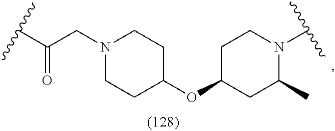 [SMILES: *C(=O)CN1CCC(O[C@H]2CCN(*)[C@@H](C)C2)CC1]

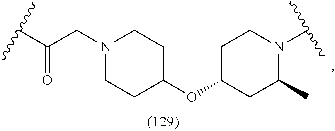 [SMILES: *C(=O)CN1CCC(O[C@@H]2CCN(*)[C@@H](C)C2)CC1]

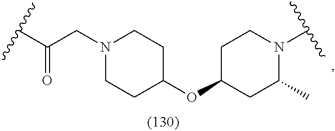 [SMILES: *C(=O)CN1CCC(O[C@H]2CCN(*)[C@H](C)C2)CC1]

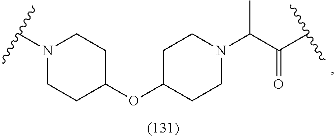 [SMILES: *C(=O)C(C)N1CCC(OC2CCN(*)CC2)CC1]

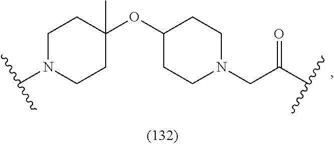 [SMILES: *C(=O)CN1CCC(OC2(C)CCN(*)CC2)CC1]

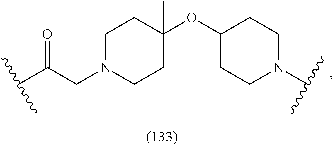 [SMILES: *C(=O)CN1CCC(C)(OC2CCN(*)CC2)CC1]

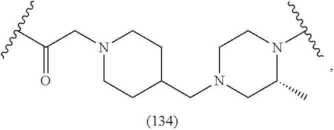 [SMILES: *C(=O)CN1CCC(CN2CCN(*)[C@H](C)C2)CC1]

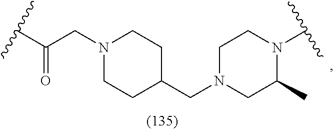 [SMILES: *C(=O)CN1CCC(CN2CCN(*)[C@@H](C)C2)CC1]

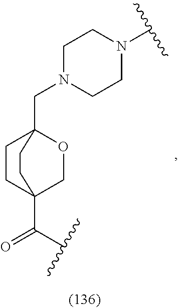 [SMILES: *C(=O)C12CCC(CN3CCN(*)CC3)(CC1)OC2]

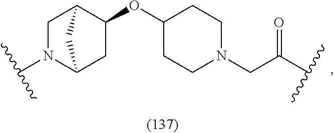 [SMILES: *C(=O)CN1CCC(O[C@H]2C[C@@H]3C[C@H]2CN3*)CC1]

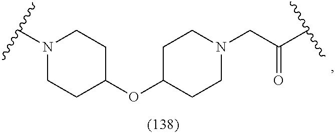 [SMILES: *C(=O)CN1CCC(OC2CCN(*)CC2)CC1]

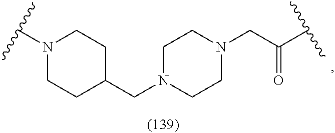 [SMILES: *C(=O)CN1CCN(CC2CCN(*)CC2)CC1]

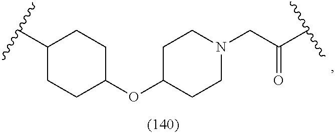 [SMILES: *C(=O)CN1CCC(OC2CCC(*)CC2)CC1]

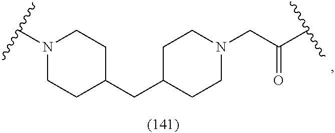 [SMILES: *C(=O)CN1CCC(CC2CCN(*)CC2)CC1]

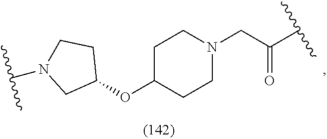 [SMILES: *C(=O)CN1CCC(O[C@H]2CCN(*)C2)CC1]

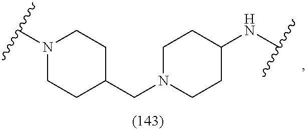 [SMILES: *NC1CCN(CC2CCN(*)CC2)CC1]

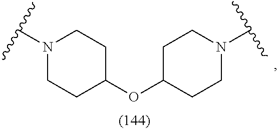 [SMILES: *N1CCC(OC2CCN(*)CC2)CC1]

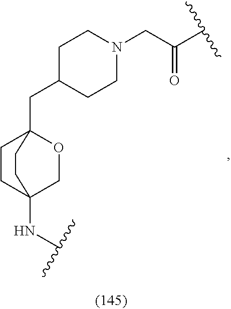 [SMILES: *NC12CCC(CC3CCN(CC(*)=O)CC3)(CC1)OC2]

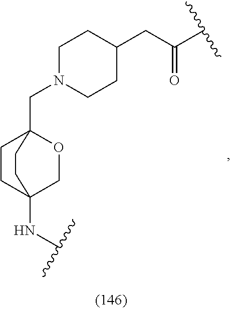 [SMILES: *NC12CCC(CN3CCC(CC(*)=O)CC3)(CC1)OC2]

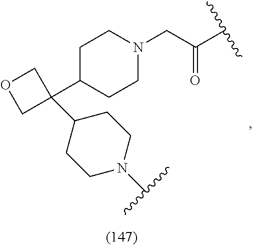 [SMILES: *C(=O)CN1CCC(C2(C3CCN(*)CC3)COC2)CC1]

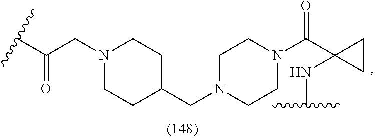 [SMILES: *NC1(C(=O)N2CCN(CC3CCN(CC(*)=O)CC3)CC2)CC1]

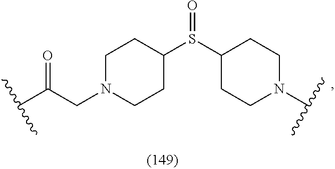 [SMILES: *C(=O)CN1CCC(S(=O)C2CCN(*)CC2)CC1]

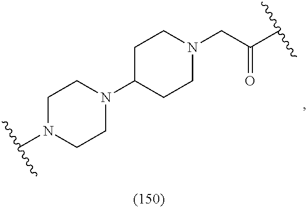 [SMILES: *C(=O)CN1CCC(N2CCN(*)CC2)CC1]

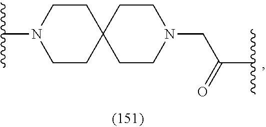 [SMILES: *C(=O)CN1CCC2(CCN(*)CC2)CC1]

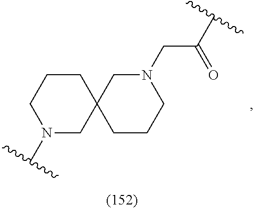 [SMILES: *C(=O)CN1CCCC2(CCCN(*)C2)C1]

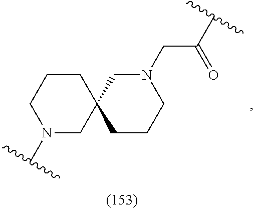 [SMILES: *C(=O)CN1CCC[C@@]2(CCCN(*)C2)C1]

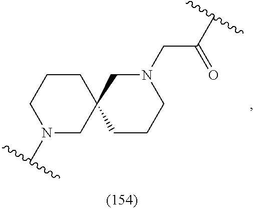 [SMILES: *C(=O)CN1CCC[C@]2(CCCN(*)C2)C1]

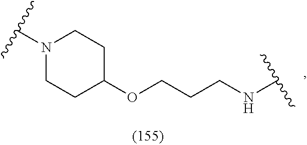 [SMILES: *NCCCOC1CCN(*)CC1]

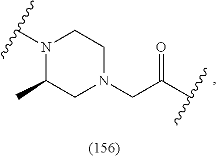 [SMILES: *C(=O)CN1CCN(*)[C@H](C)C1]

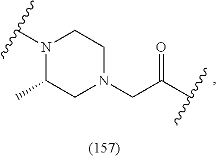 [SMILES: *C(=O)CN1CCN(*)[C@@H](C)C1]

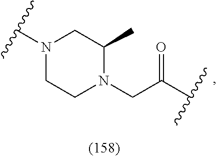 [SMILES: *C(=O)CN1CCN(*)C[C@H]1C]

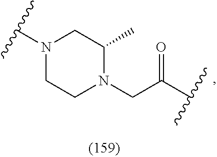 [SMILES: *C(=O)CN1CCN(*)C[C@@H]1C]

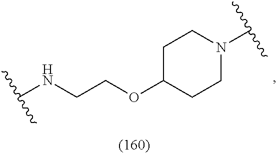 [SMILES: *NCCOC1CCN(*)CC1]

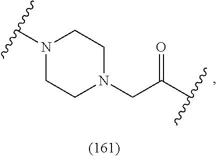 [SMILES: *C(=O)CN1CCN(*)CC1]

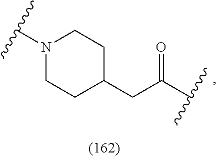 [SMILES: *C(=O)CC1CCN(*)CC1]

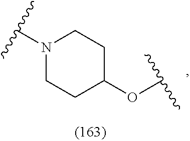 [SMILES: *OC1CCN(*)CC1]

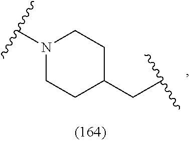 [SMILES: *CC1CCN(*)CC1]

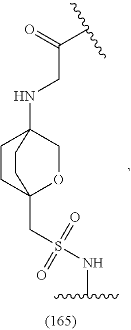 [SMILES: *NS(=O)(=O)CC12CCC(NCC(*)=O)(CC1)CO2]

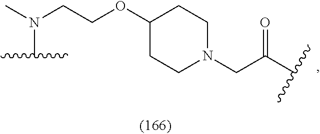 [SMILES: *C(=O)CN1CCC(OCCN(*)C)CC1]

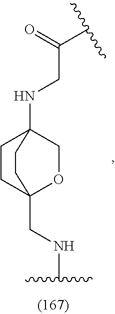 [SMILES: *NCC12CCC(NCC(*)=O)(CC1)CO2]

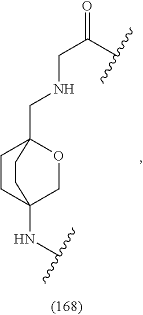 [SMILES: *NC12CCC(CNCC(*)=O)(CC1)OC2]

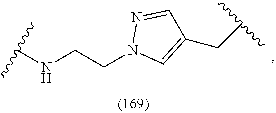 [SMILES: *Cc1cnn(CCN*)c1]

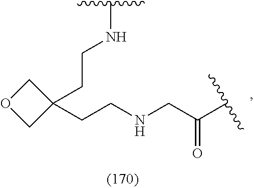 [SMILES: *NCCC1(CCNCC(*)=O)COC1]

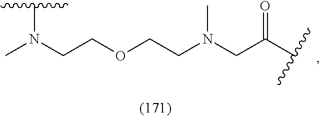 [SMILES: *C(=O)CN(C)CCOCCN(*)C]

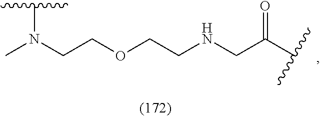 [SMILES: *C(=O)CNCCOCCN(*)C]

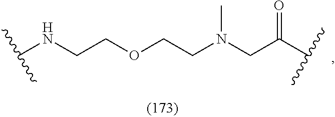 [SMILES: *NCCOCCN(C)CC(*)=O]

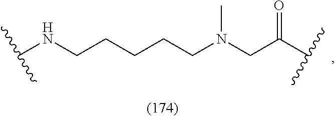 [SMILES: *NCCCCCN(C)CC(*)=O]

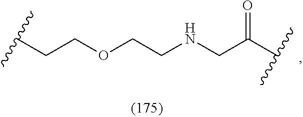 [SMILES: *CCOCCNCC(*)=O]

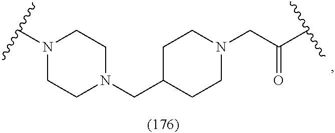 [SMILES: *C(=O)CN1CCC(CN2CCN(*)CC2)CC1]

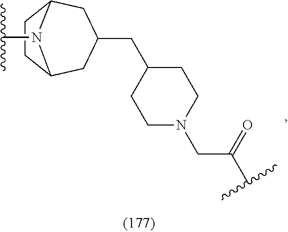 [SMILES: *C(=O)CN1CCC(CC2CC3CCC(C2)N3*)CC1]

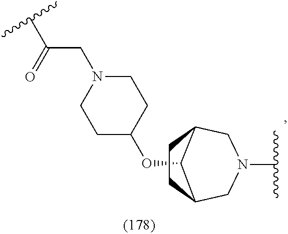 [SMILES: *C(=O)CN1CCC(O[C@H]2[C@@H]3CC[C@H]2CN(*)C3)CC1]

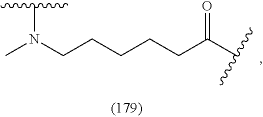 [SMILES: *C(=O)CCCCCN(*)C]

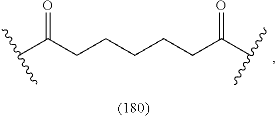 [SMILES: *C(=O)CCCCCC(*)=O]

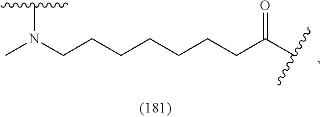 [SMILES: *C(=O)CCCCCCCN(*)C]

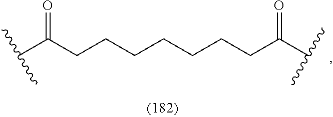 [SMILES: *C(=O)CCCCCCCC(*)=O]

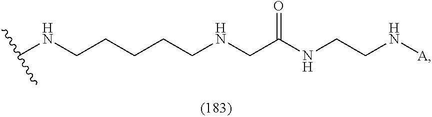

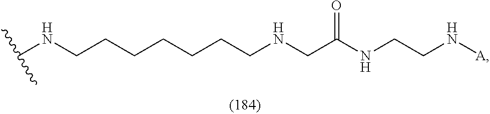

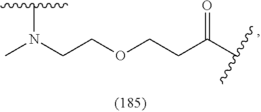 [SMILES: *C(=O)CCOCCN(*)C]

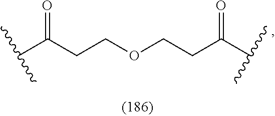 [SMILES: *C(=O)CCOCCC(*)=O]

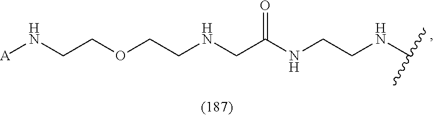

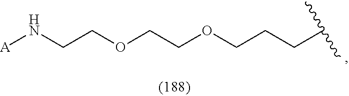

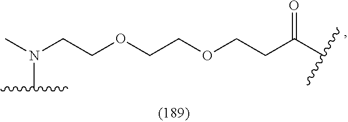 [SMILES: *C(=O)CCOCCOCCN(*)C]

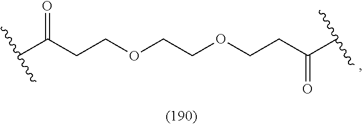 [SMILES: *C(=O)CCOCCOCCC(*)=O]

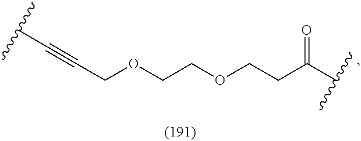 [SMILES: *C#CCOCCOCCC(*)=O]

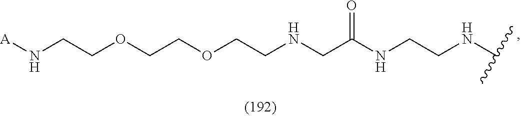

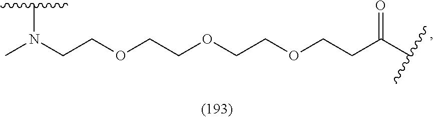 [SMILES: *C(=O)CCOCCOCCOCCN(*)C]

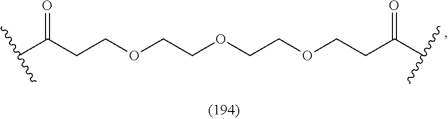 [SMILES: *C(=O)CCOCCOCCOCCC(*)=O]

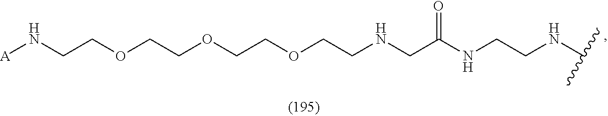

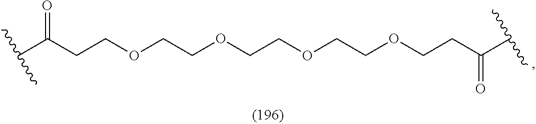 [SMILES: *C(=O)CCOCCOCCOCCOCCC(*)=O]

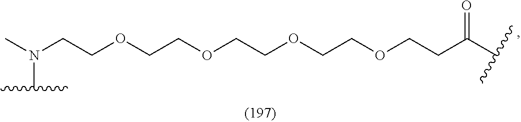 [SMILES: *C(=O)CCOCCOCCOCCOCCN(*)C]

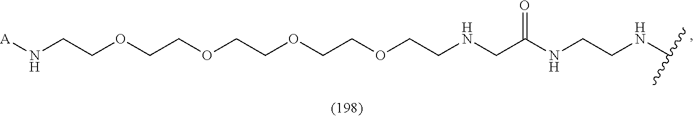

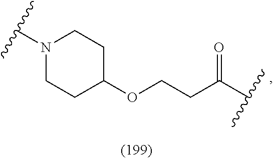 [SMILES: *C(=O)CCOC1CCN(*)CC1]

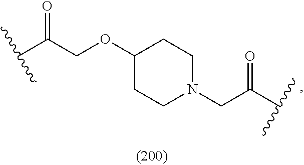 [SMILES: *C(=O)COC1CCN(CC(*)=O)CC1]

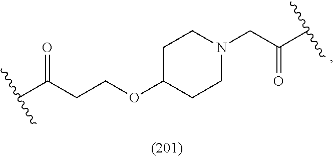 [SMILES: *C(=O)CCOC1CCN(CC(*)=O)CC1]

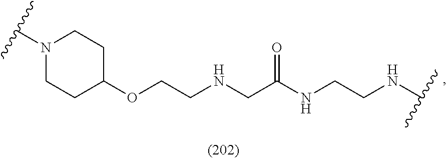 [SMILES: *NCCNC(=O)CNCCOC1CCN(*)CC1]

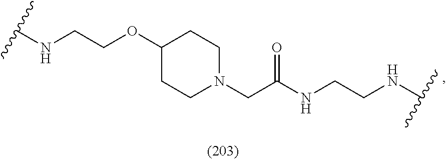 [SMILES: *NCCNC(=O)CN1CCC(OCCN*)CC1]

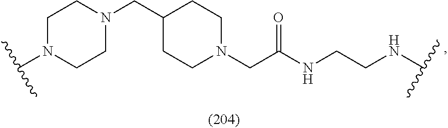 [SMILES: *NCCNC(=O)CN1CCC(CN2CCN(*)CC2)CC1]

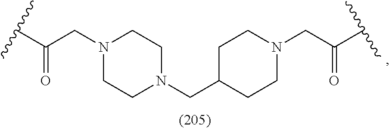 [SMILES: *C(=O)CN1CCC(CN2CCN(CC(*)=O)CC2)CC1]

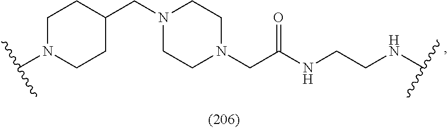 [SMILES: *NCCNC(=O)CN1CCN(CC2CCN(*)CC2)CC1]

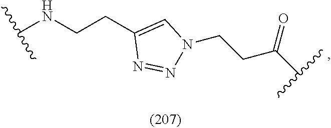 [SMILES: *NCCc1cn(CCC(*)=O)nn1]

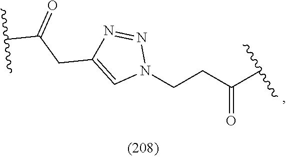 [SMILES: *C(=O)CCn1cc(CC(*)=O)nn1]

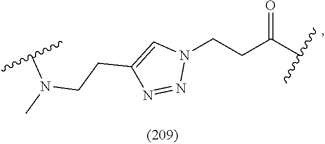 [SMILES: *C(=O)CCn1cc(CCN(*)C)nn1]

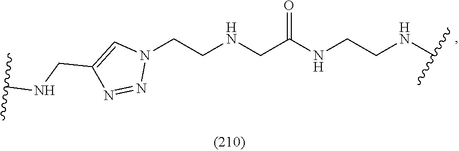 [SMILES: *NCCNC(=O)CNCCn1cc(CN*)nn1]

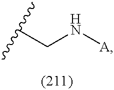

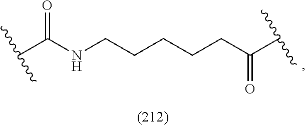 [SMILES: *C(=O)CCCCCNC(*)=O]

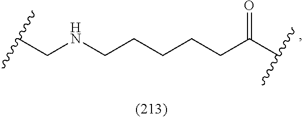 [SMILES: *CNCCCCCC(*)=O]

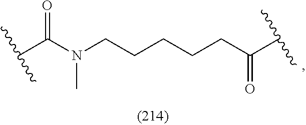 [SMILES: *C(=O)CCCCCN(C)C(*)=O]

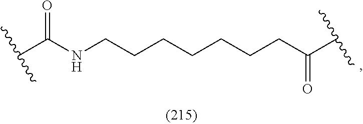 [SMILES: *C(=O)CCCCCCCNC(*)=O]

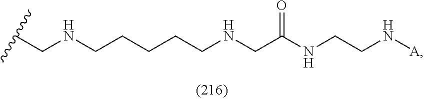

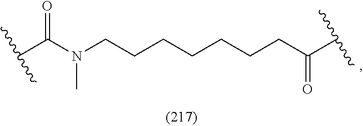 [SMILES: *C(=O)CCCCCCCN(C)C(*)=O]

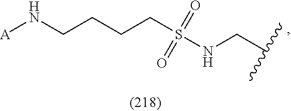

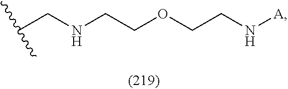

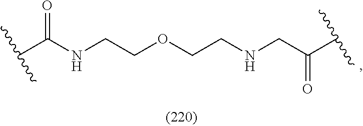 [SMILES: *C(=O)CNCCOCCNC(*)=O]

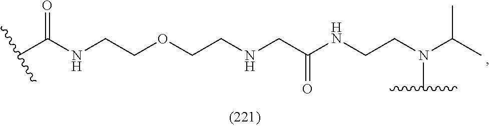 [SMILES: *C(=O)NCCOCCNCC(=O)NCCN(*)C(C)C]

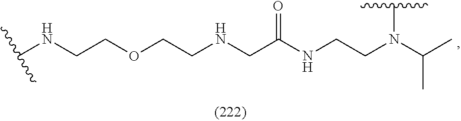 [SMILES: *NCCOCCNCC(=O)NCCN(*)C(C)C]

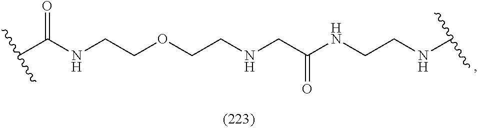 [SMILES: *NCCNC(=O)CNCCOCCNC(*)=O]

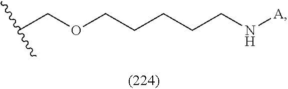

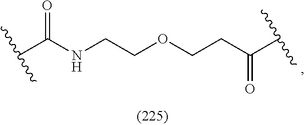 [SMILES: *C(=O)CCOCCNC(*)=O]

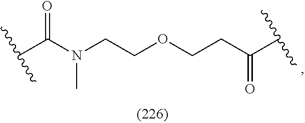 [SMILES: *C(=O)CCOCCN(C)C(*)=O]

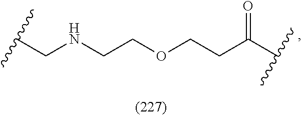 [SMILES: *CNCCOCCC(*)=O]

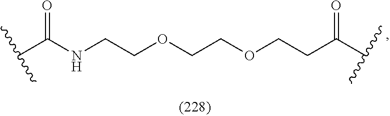 [SMILES: *C(=O)CCOCCOCCNC(*)=O]

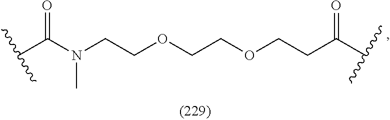 [SMILES: *C(=O)CCOCCOCCN(C)C(*)=O]

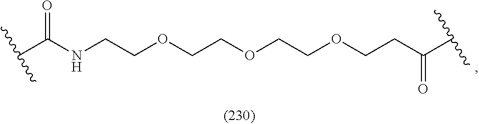 [SMILES: *C(=O)CCOCCOCCOCCNC(*)=O]

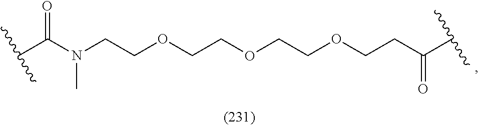 [SMILES: *C(=O)CCOCCOCCOCCN(C)C(*)=O]

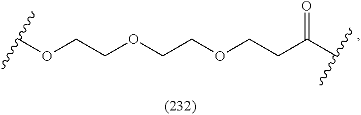 [SMILES: *OCCOCCOCCC(*)=O]

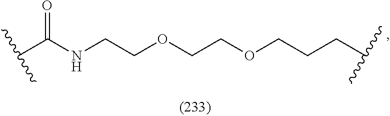 [SMILES: *CCCOCCOCCNC(*)=O]

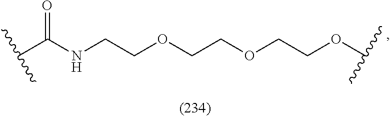 [SMILES: *OCCOCCOCCNC(*)=O]

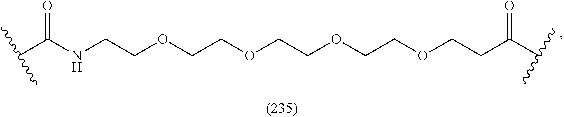 [SMILES: *C(=O)CCOCCOCCOCCOCCNC(*)=O]

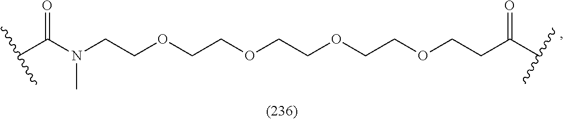 [SMILES: *C(=O)CCOCCOCCOCCOCCN(C)C(*)=O]

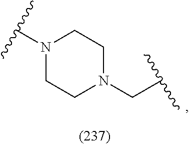 [SMILES: *CN1CCN(*)CC1]

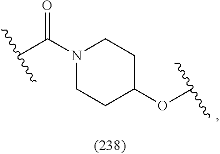 [SMILES: *OC1CCN(C(*)=O)CC1]

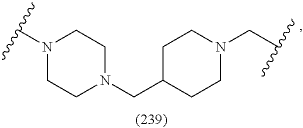 [SMILES: *CN1CCC(CN2CCN(*)CC2)CC1]

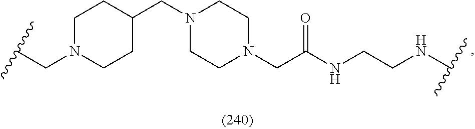 [SMILES: *CN1CCC(CN2CCN(CC(=O)NCCN*)CC2)CC1]

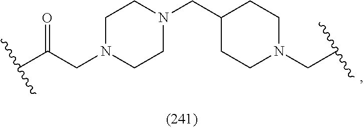 [SMILES: *CN1CCC(CN2CCN(CC(*)=O)CC2)CC1]

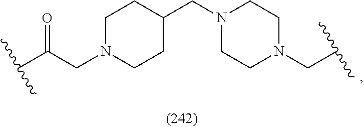 [SMILES: *CN1CCN(CC2CCN(CC(*)=O)CC2)CC1]

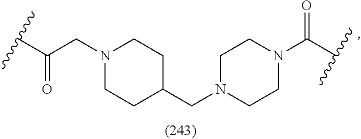 [SMILES: *C(=O)CN1CCC(CN2CCN(C(*)=O)CC2)CC1]

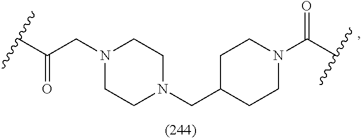 [SMILES: *C(=O)CN1CCN(CC2CCN(C(*)=O)CC2)CC1]

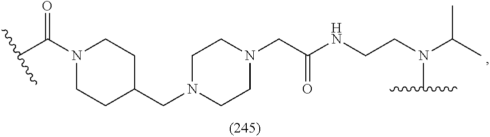 [SMILES: *C(=O)N1CCC(CN2CCN(CC(=O)NCCN(*)C(C)C)CC2)CC1]

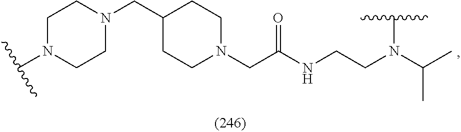 [SMILES: *N1CCN(CC2CCN(CC(=O)NCCN(*)C(C)C)CC2)CC1]

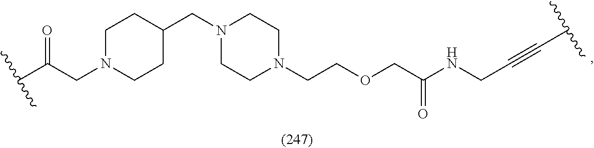 [SMILES: *C#CCNC(=O)COCCN1CCN(CC2CCN(CC(*)=O)CC2)CC1]

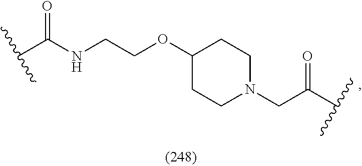 [SMILES: *C(=O)CN1CCC(OCCNC(*)=O)CC1]

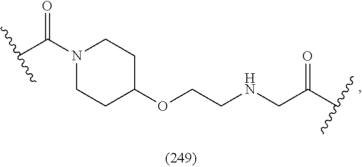 [SMILES: *C(=O)CNCCOC1CCN(C(*)=O)CC1]

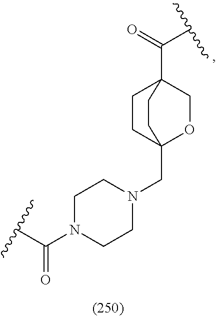 [SMILES: *C(=O)N1CCN(CC23CCC(C(*)=O)(CC2)CO3)CC1]

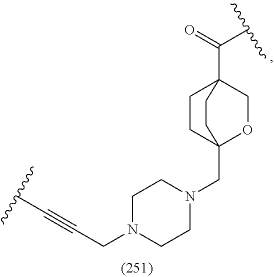 [SMILES: *C#CCN1CCN(CC23CCC(C(*)=O)(CC2)CO3)CC1]

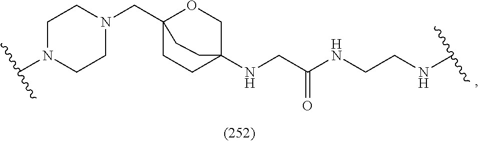 [SMILES: *NCCNC(=O)CNC12CCC(CN3CCN(*)CC3)(CC1)OC2]

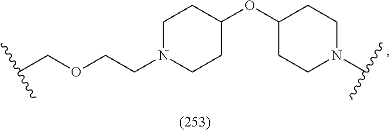 [SMILES: *COCCN1CCC(OC2CCN(*)CC2)CC1]

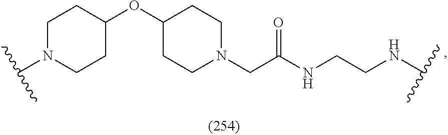 [SMILES: *NCCNC(=O)CN1CCC(OC2CCN(*)CC2)CC1]

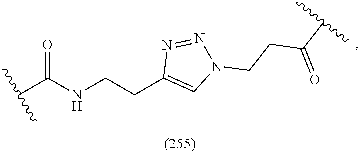 [SMILES: *C(=O)CCn1cc(CCNC(*)=O)nn1]

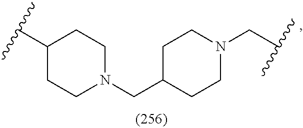 [SMILES: *CN1CCC(CN2CCC(*)CC2)CC1]

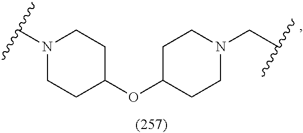 [SMILES: *CN1CCC(OC2CCN(*)CC2)CC1]

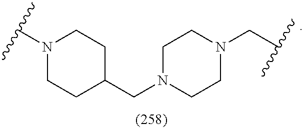 [SMILES: *CN1CCN(CC2CCN(*)CC2)CC1]

In some embodiments, the present invention provides a compound having PBM described and disclosed herein. DBM set forth in Table A above, and a linker set forth in Table B above, or a pharmaceutically acceptable salt thereof.

Exemplary compounds of the invention are set forth in Table 1, below:

TABLE 1
Exemplary Compounds

| ID# | Structure |
|---|---|
| I-1 | 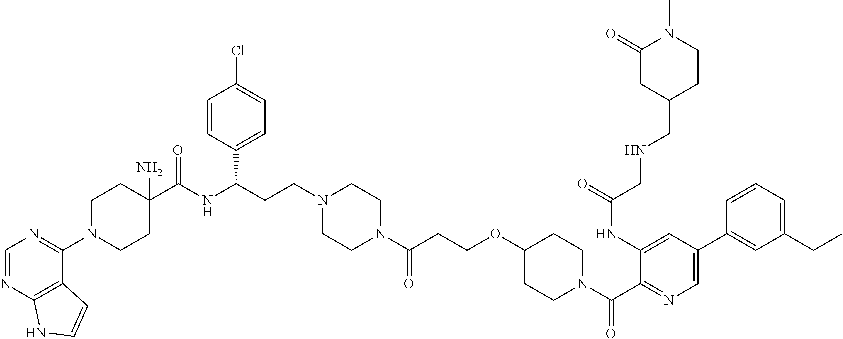 [SMILES: CCc1cccc(-c2cnc(C(=O)N3CCC(OCCC(=O)N4CCN(CC[C@H](NC(=O)C5(N)CCN(c6ncnc7[nH]ccc67)CC5)c5ccc(Cl)cc5)CC4)CC3)c(NC(=O)CNCC3CCN(C)C(=O)C3)c2)c1] |
| I-2 | 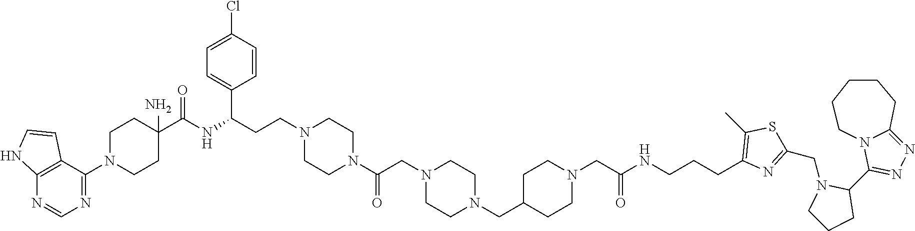 [SMILES: Cc1sc(CN2CCCC2c2nnc3n2CCCCC3)nc1CCCNC(=O)CN1CCC(CN2CCN(CC(=O)N3CCN(CC[C@H](NC(=O)C4(N)CCN(c5ncnc6[nH]ccc56)CC4)c4ccc(Cl)cc4)CC3)CC2)CC1] |
| I-3 | 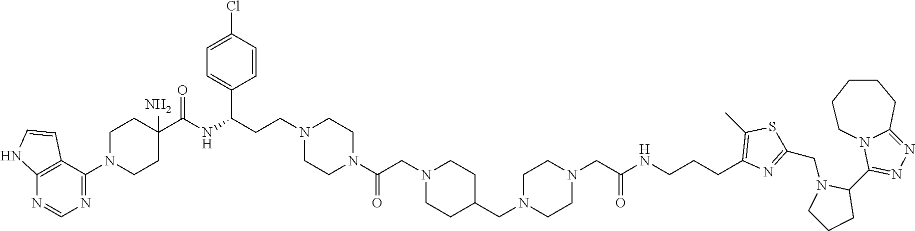 [SMILES: Cc1sc(CN2CCCC2c2nnc3n2CCCCC3)nc1CCCNC(=O)CN1CCN(CC2CCN(CC(=O)N3CCN(CC[C@H](NC(=O)C4(N)CCN(c5ncnc6[nH]ccc56)CC4)c4ccc(Cl)cc4)CC3)CC2)CC1] |
| I-4 | 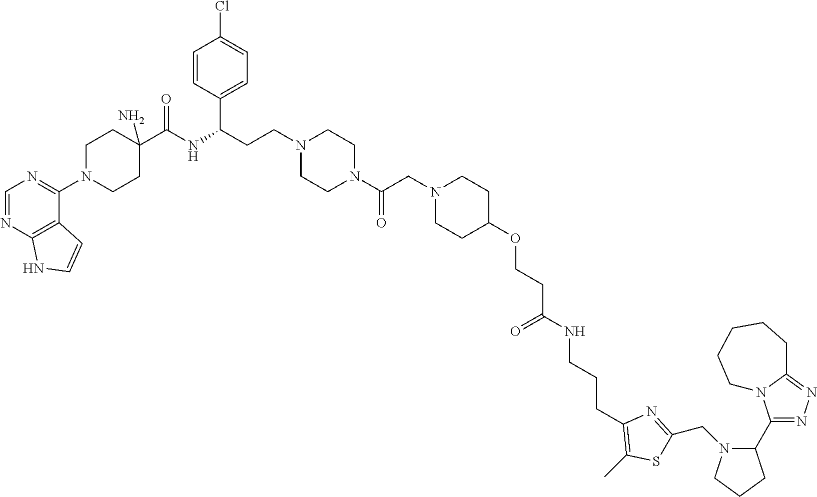 [SMILES: Cc1sc(CN2CCCC2c2nnc3n2CCCCC3)nc1CCCNC(=O)CCOC1CCN(CC(=O)N2CCN(CC[C@H](NC(=O)C3(N)CCN(c4ncnc5[nH]ccc45)CC3)c3ccc(Cl)cc3)CC2)CC1] |
| I-5 | 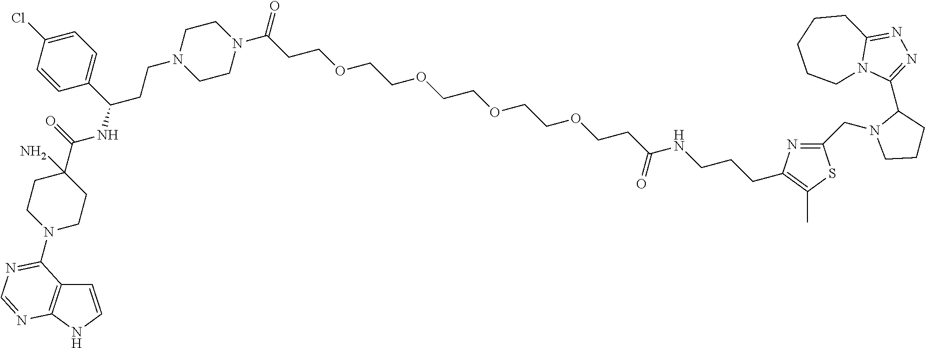 [SMILES: Cc1sc(CN2CCCC2c2nnc3n2CCCCC3)nc1CCCNC(=O)CCOCCOCCOCCOCCC(=O)N1CCN(CC[C@H](NC(=O)C2(N)CCN(c3ncnc4[nH]ccc34)CC2)c2ccc(Cl)cc2)CC1] |
| I-6 | 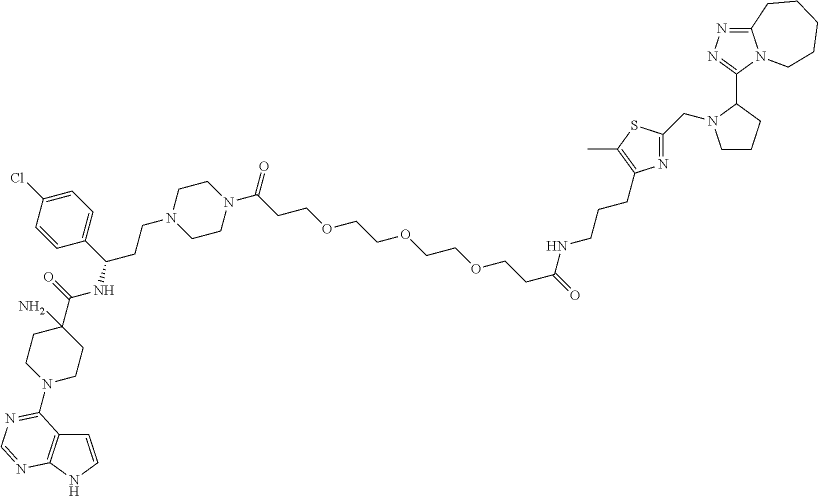 [SMILES: Cc1sc(CN2CCCC2c2nnc3n2CCCCC3)nc1CCCNC(=O)CCOCCOCCOCCC(=O)N1CCN(CC[C@H](NC(=O)C2(N)CCN(c3ncnc4[nH]ccc34)CC2)c2ccc(Cl)cc2)CC1] |
| I-7 | 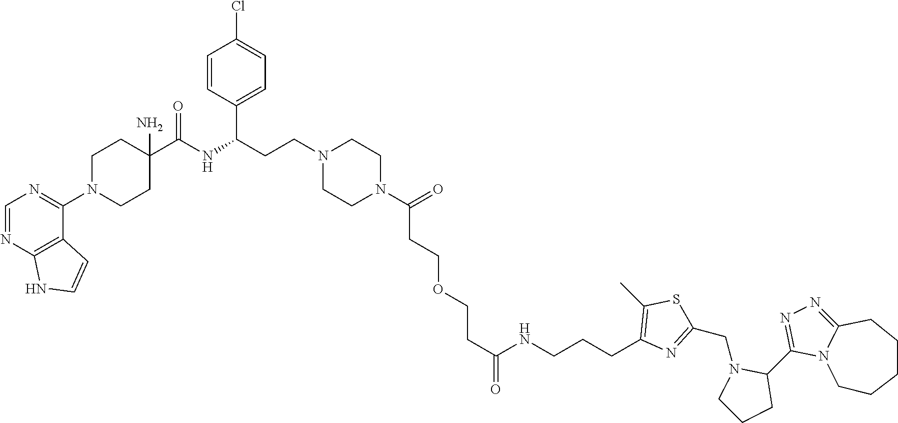 [SMILES: Cc1sc(CN2CCCC2c2nnc3n2CCCCC3)nc1CCCNC(=O)CCOCCC(=O)N1CCN(CC[C@H](NC(=O)C2(N)CCN(c3ncnc4[nH]ccc34)CC2)c2ccc(Cl)cc2)CC1] |
| I-8 | 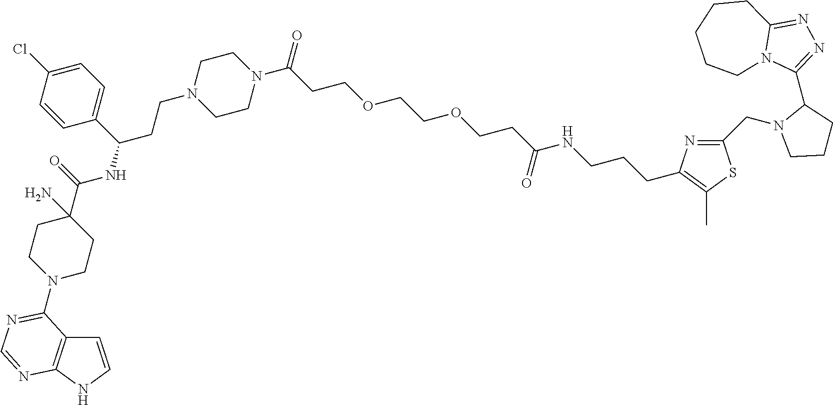 [SMILES: Cc1sc(CN2CCCC2c2nnc3n2CCCCC3)nc1CCCNC(=O)CCOCCOCCC(=O)N1CCN(CC[C@H](NC(=O)C2(N)CCN(c3ncnc4[nH]ccc34)CC2)c2ccc(Cl)cc2)CC1] |
| I-9 | 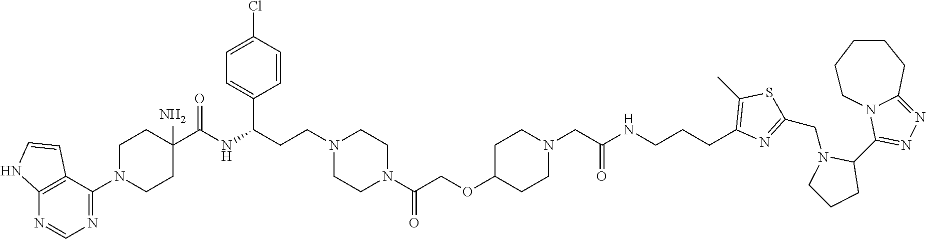 [SMILES: Cc1sc(CN2CCCC2c2nnc3n2CCCCC3)nc1CCCNC(=O)CN1CCC(OCC(=O)N2CCN(CC[C@H](NC(=O)C3(N)CCN(c4ncnc5[nH]ccc45)CC3)c3ccc(Cl)cc3)CC2)CC1] |
| I-10 | 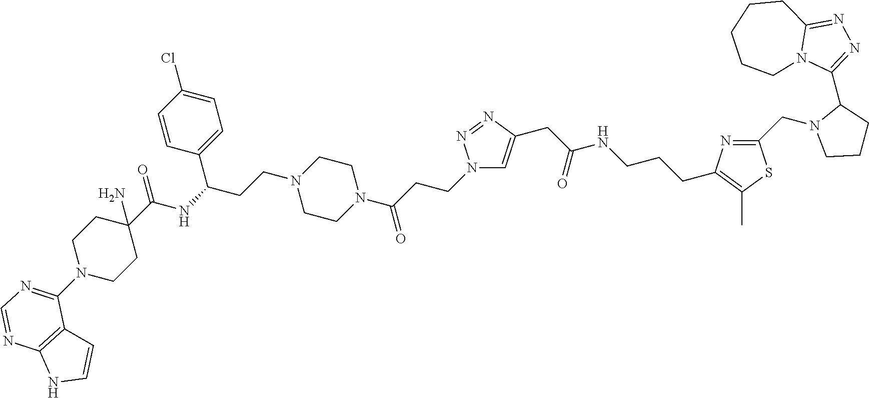 [SMILES: Cc1sc(CN2CCCC2c2nnc3n2CCCCC3)nc1CCCNC(=O)Cc1cn(CCC(=O)N2CCN(CC[C@H](NC(=O)C3(N)CCN(c4ncnc5[nH]ccc45)CC3)c3ccc(Cl)cc3)CC2)nn1] |
| I-11 | 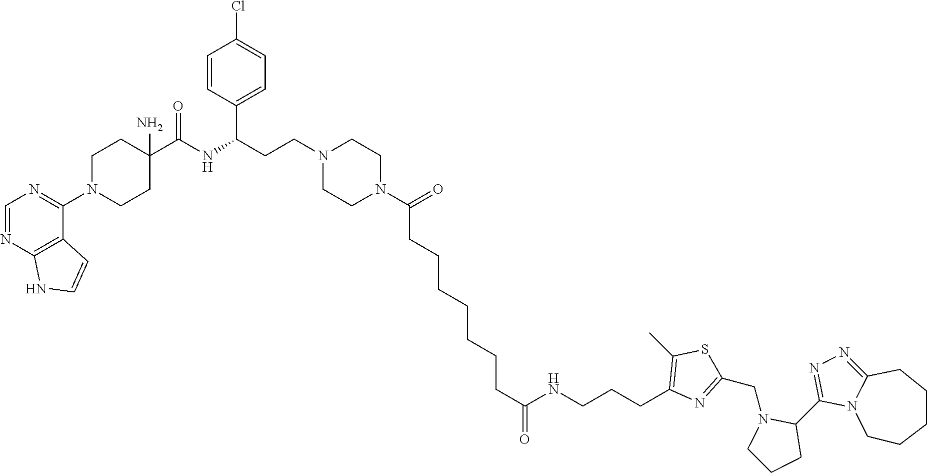 [SMILES: Cc1sc(CN2CCCC2c2nnc3n2CCCCC3)nc1CCCNC(=O)CCCCCCCC(=O)N1CCN(CC[C@H](NC(=O)C2(N)CCN(c3ncnc4[nH]ccc34)CC2)c2ccc(Cl)cc2)CC1] |
| I-12 | 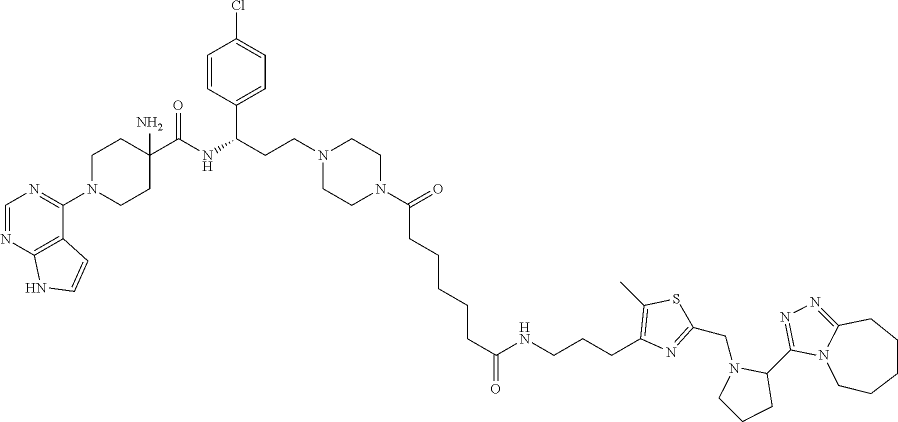 [SMILES: Cc1sc(CN2CCCC2c2nnc3n2CCCCC3)nc1CCCNC(=O)CCCCCC(=O)N1CCN(CC[C@H](NC(=O)C2(N)CCN(c3ncnc4[nH]ccc34)CC2)c2ccc(Cl)cc2)CC1] |
| I-13 | 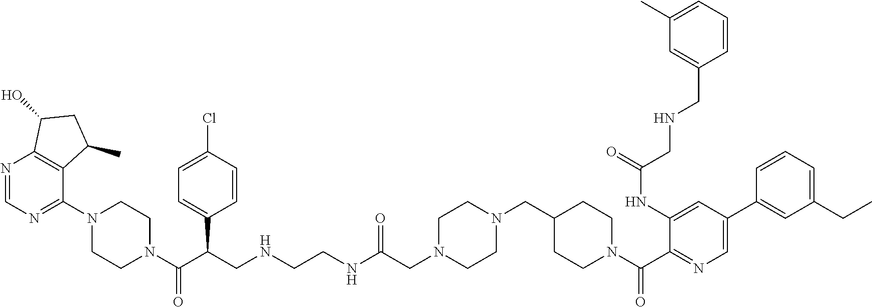 [SMILES: CCc1cccc(-c2cnc(C(=O)N3CCC(CN4CCN(CC(=O)NCCNC[C@@H](C(=O)N5CCN(c6ncnc7c6[C@H](C)C[C@H]7O)CC5)c5ccc(Cl)cc5)CC4)CC3)c(NC(=O)CNCc3cccc(C)c3)c2)c1] |
| I-14 | 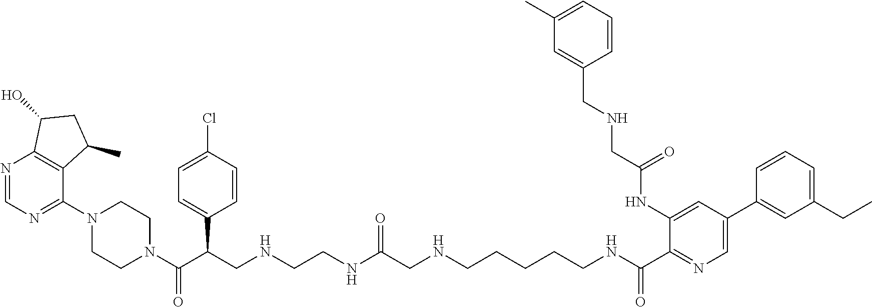 [SMILES: CCc1cccc(-c2cnc(C(=O)NCCCCCNCC(=O)NCCNC[C@@H](C(=O)N3CCN(c4ncnc5c4[C@H](C)C[C@H]5O)CC3)c3ccc(Cl)cc3)c(NC(=O)CNCc3cccc(C)c3)c2)c1] |
| I-15 | 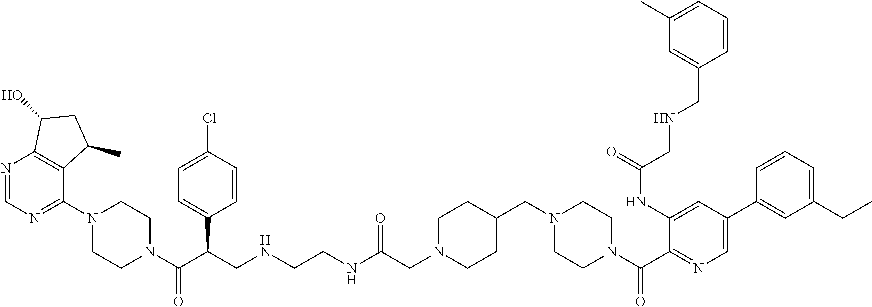 [SMILES: CCc1cccc(-c2cnc(C(=O)N3CCN(CC4CCN(CC(=O)NCCNC[C@@H](C(=O)N5CCN(c6ncnc7c6[C@H](C)C[C@H]7O)CC5)c5ccc(Cl)cc5)CC4)CC3)c(NC(=O)CNCc3cccc(C)c3)c2)c1] |
| I-16 | 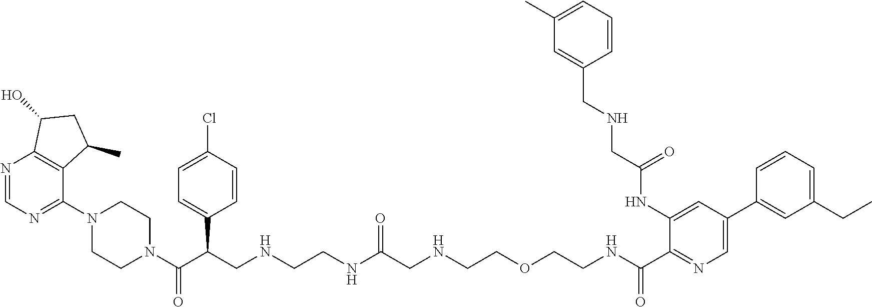 [SMILES: CCc1cccc(-c2cnc(C(=O)NCCOCCNCC(=O)NCCNC[C@@H](C(=O)N3CCN(c4ncnc5c4[C@H](C)C[C@H]5O)CC3)c3ccc(Cl)cc3)c(NC(=O)CNCc3cccc(C)c3)c2)c1] |
| I-17 | 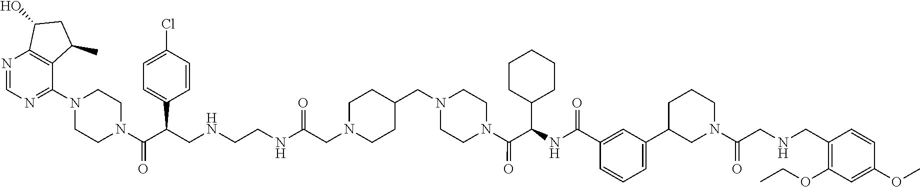 [SMILES: CCOc1cc(OC)ccc1CNCC(=O)N1CCCC(c2cccc(C(=O)N[C@@H](C(=O)N3CCN(CC4CCN(CC(=O)NCCNC[C@@H](C(=O)N5CCN(c6ncnc7c6[C@H](C)C[C@H]7O)CC5)c5ccc(Cl)cc5)CC4)CC3)C3CCCCC3)c2)C1] |
| I-18 | 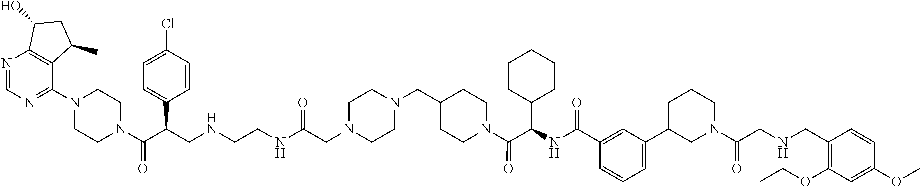 [SMILES: CCOc1cc(OC)ccc1CNCC(=O)N1CCCC(c2cccc(C(=O)N[C@@H](C(=O)N3CCC(CN4CCN(CC(=O)NCCNC[C@@H](C(=O)N5CCN(c6ncnc7c6[C@H](C)C[C@H]7O)CC5)c5ccc(Cl)cc5)CC4)CC3)C3CCCCC3)c2)C1] |
| I-19 | 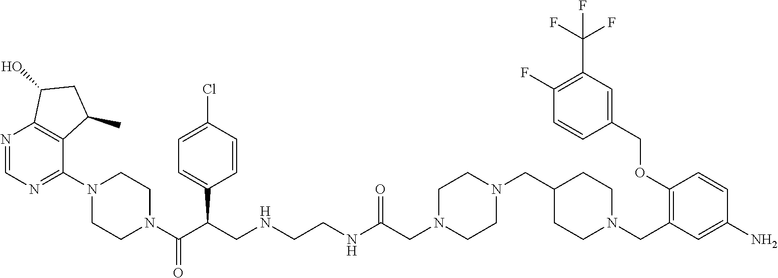 [SMILES: C[C@@H]1C[C@@H](O)c2ncnc(N3CCN(C(=O)[C@H](CNCCNC(=O)CN4CCN(CC5CCN(Cc6cc(N)ccc6OCc6ccc(F)c(C(F)(F)F)c6)CC5)CC4)c4ccc(Cl)cc4)CC3)c21] |
| I-20 | 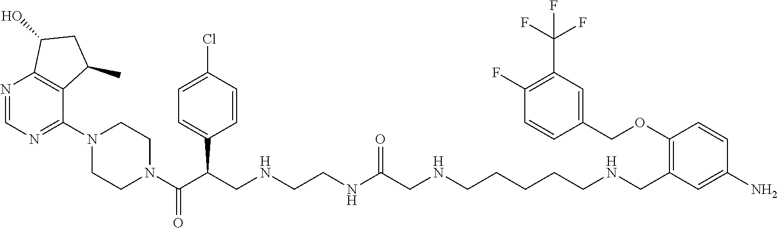 [SMILES: C[C@@H]1C[C@@H](O)c2ncnc(N3CCN(C(=O)[C@H](CNCCNC(=O)CNCCCCCNCc4cc(N)ccc4OCc4ccc(F)c(C(F)(F)F)c4)c4ccc(Cl)cc4)CC3)c21] |
| I-21 | 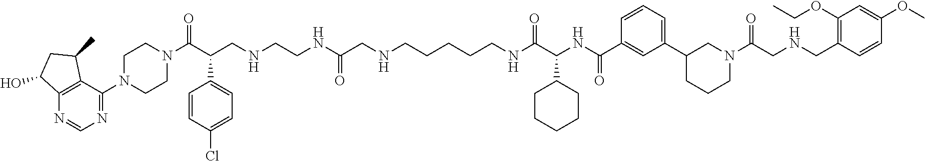 [SMILES: CCOc1cc(OC)ccc1CNCC(=O)N1CCCC(c2cccc(C(=O)N[C@@H](C(=O)NCCCCCNCC(=O)NCCNC[C@@H](C(=O)N3CCN(c4ncnc5c4[C@H](C)C[C@H]5O)CC3)c3ccc(Cl)cc3)C3CCCCC3)c2)C1] |
| I-22 | 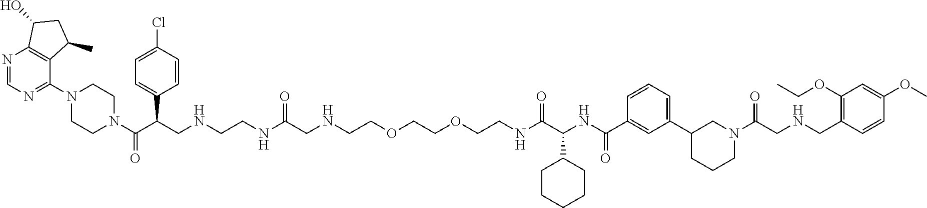 [SMILES: CCOc1cc(OC)ccc1CNCC(=O)N1CCCC(c2cccc(C(=O)N[C@@H](C(=O)NCCOCCOCCNCC(=O)NCCNC[C@@H](C(=O)N3CCN(c4ncnc5c4[C@H](C)C[C@H]5O)CC3)c3ccc(Cl)cc3)C3CCCCC3)c2)C1] |
| I-23 | 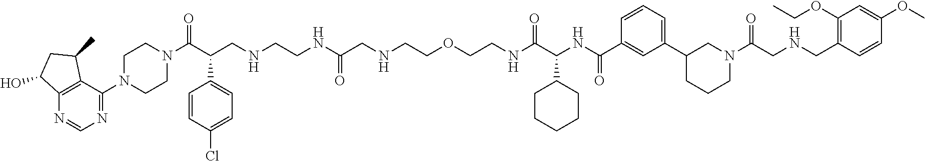 [SMILES: CCOc1cc(OC)ccc1CNCC(=O)N1CCCC(c2cccc(C(=O)N[C@@H](C(=O)NCCOCCNCC(=O)NCCNC[C@@H](C(=O)N3CCN(c4ncnc5c4[C@H](C)C[C@H]5O)CC3)c3ccc(Cl)cc3)C3CCCCC3)c2)C1] |
| I-24 | 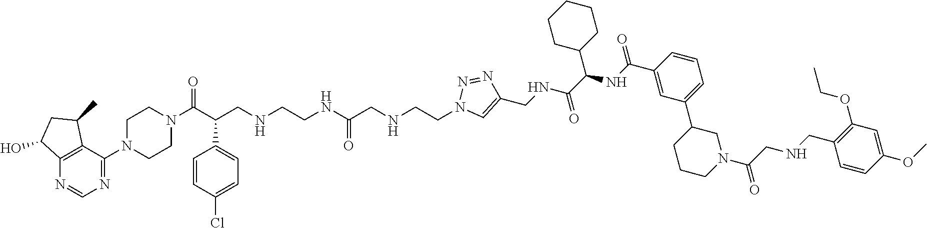 [SMILES: CCOc1cc(OC)ccc1CNCC(=O)N1CCCC(c2cccc(C(=O)N[C@@H](C(=O)NCc3cn(CCNCC(=O)NCCNC[C@@H](C(=O)N4CCN(c5ncnc6c5[C@H](C)C[C@H]6O)CC4)c4ccc(Cl)cc4)nn3)C3CCCCC3)c2)C1] |
| I-25 | 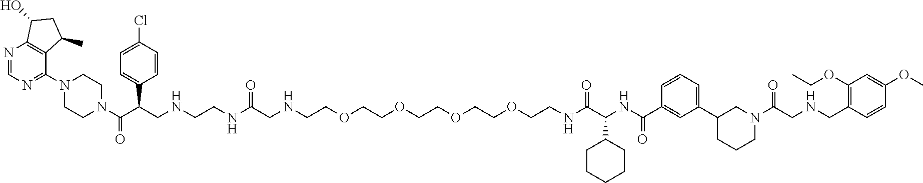 [SMILES: CCOc1cc(OC)ccc1CNCC(=O)N1CCCC(c2cccc(C(=O)N[C@@H](C(=O)NCCOCCOCCOCCOCCNCC(=O)NCCNC[C@@H](C(=O)N3CCN(c4ncnc5c4[C@H](C)C[C@H]5O)CC3)c3ccc(Cl)cc3)C3CCCCC3)c2)C1] |
| I-26 | 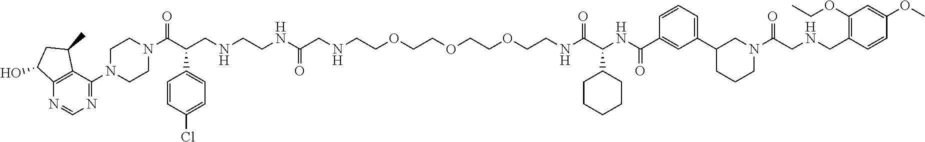 [SMILES: CCOc1cc(OC)ccc1CNCC(=O)N1CCCC(c2cccc(C(=O)N[C@@H](C(=O)NCCOCCOCCOCCNCC(=O)NCCNC[C@@H](C(=O)N3CCN(c4ncnc5c4[C@H](C)C[C@H]5O)CC3)c3ccc(Cl)cc3)C3CCCCC3)c2)C1] |
| I-27 | 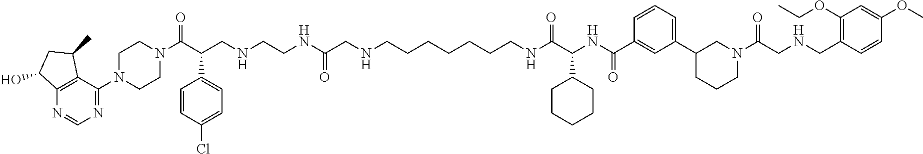 [SMILES: CCOc1cc(OC)ccc1CNCC(=O)N1CCCC(c2cccc(C(=O)N[C@@H](C(=O)NCCCCCCCNCC(=O)NCCNC[C@@H](C(=O)N3CCN(c4ncnc5c4[C@H](C)C[C@H]5O)CC3)c3ccc(Cl)cc3)C3CCCCC3)c2)C1] |
| I-28 | 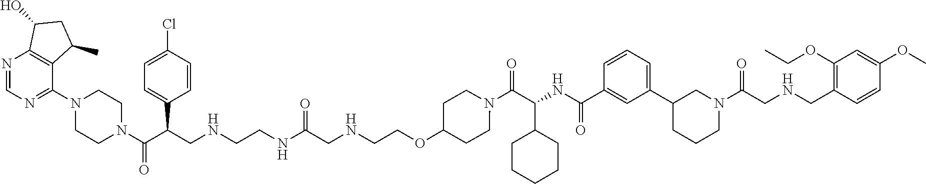 [SMILES: CCOc1cc(OC)ccc1CNCC(=O)N1CCCC(c2cccc(C(=O)N[C@@H](C(=O)N3CCC(OCCNCC(=O)NCCNC[C@@H](C(=O)N4CCN(c5ncnc6c5[C@H](C)C[C@H]6O)CC4)c4ccc(Cl)cc4)CC3)C3CCCCC3)c2)C1] |
| I-29 | 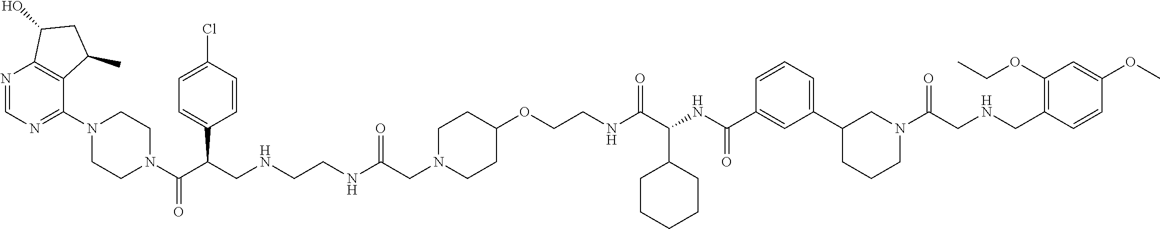 [SMILES: CCOc1cc(OC)ccc1CNCC(=O)N1CCCC(c2cccc(C(=O)N[C@@H](C(=O)NCCOC3CCN(CC(=O)NCCNC[C@@H](C(=O)N4CCN(c5ncnc6c5[C@H](C)C[C@H]6O)CC4)c4ccc(Cl)cc4)CC3)C3CCCCC3)c2)C1] |
| I-30 | 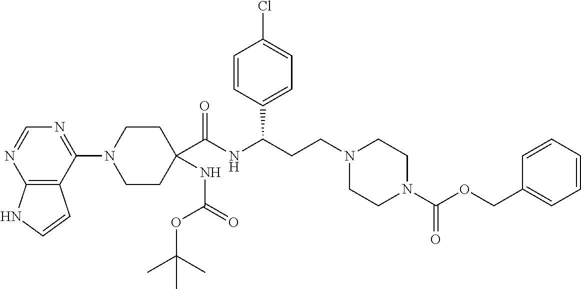 [SMILES: CC(C)(C)OC(=O)NC1(C(=O)N[C@@H](CCN2CCN(C(=O)OCc3ccccc3)CC2)c2ccc(Cl)cc2)CCN(c2ncnc3[nH]ccc23)CC1] |
| I-31 | 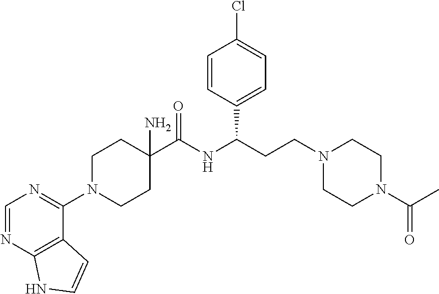 [SMILES: CC(=O)N1CCN(CC[C@H](NC(=O)C2(N)CCN(c3ncnc4[nH]ccc34)CC2)c2ccc(Cl)cc2)CC1] |
| I-32 | 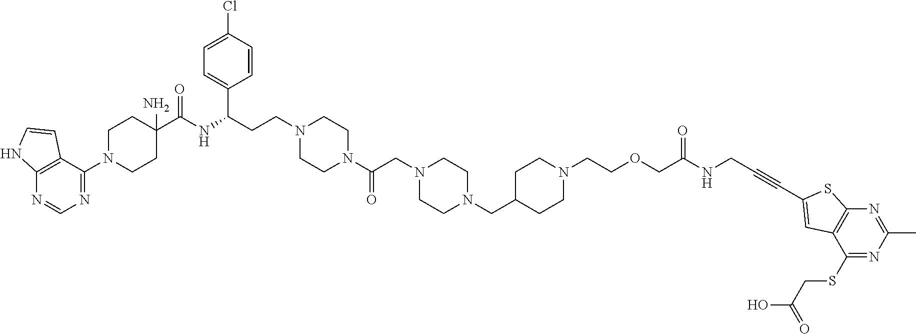 [SMILES: Cc1nc(SCC(=O)O)c2cc(C#CCNC(=O)COCCN3CCC(CN4CCN(CC(=O)N5CCN(CC[C@H](NC(=O)C6(N)CCN(c7ncnc8[nH]ccc78)CC6)c6ccc(Cl)cc6)CC5)CC4)CC3)sc2n1] |
| I-33 | 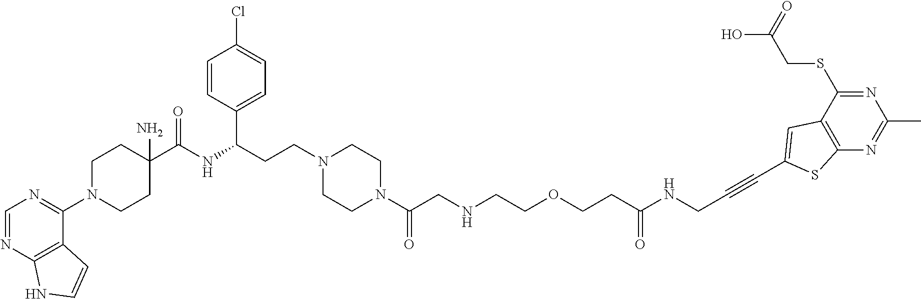 [SMILES: Cc1nc(SCC(=O)O)c2cc(C#CCNC(=O)CCOCCNCC(=O)N3CCN(CC[C@H](NC(=O)C4(N)CCN(c5ncnc6[nH]ccc56)CC4)c4ccc(Cl)cc4)CC3)sc2n1] |
| I-34 | 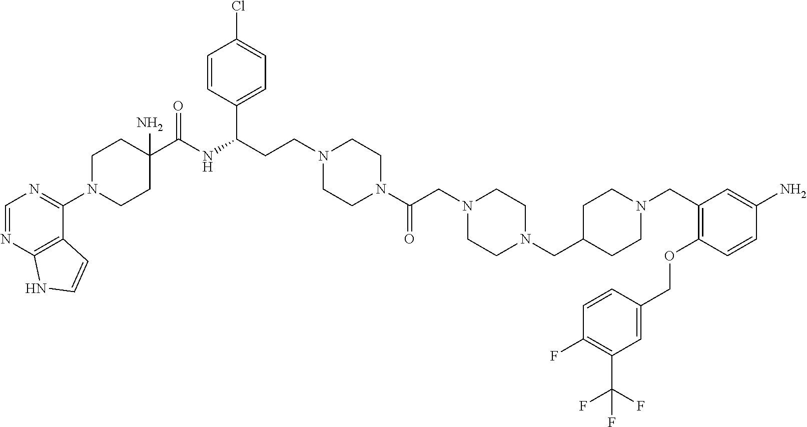 [SMILES: Nc1ccc(OCc2ccc(F)c(C(F)(F)F)c2)c(CN2CCC(CN3CCN(CC(=O)N4CCN(CC[C@H](NC(=O)C5(N)CCN(c6ncnc7[nH]ccc67)CC5)c5ccc(Cl)cc5)CC4)CC3)CC2)c1] |
| I-35 | 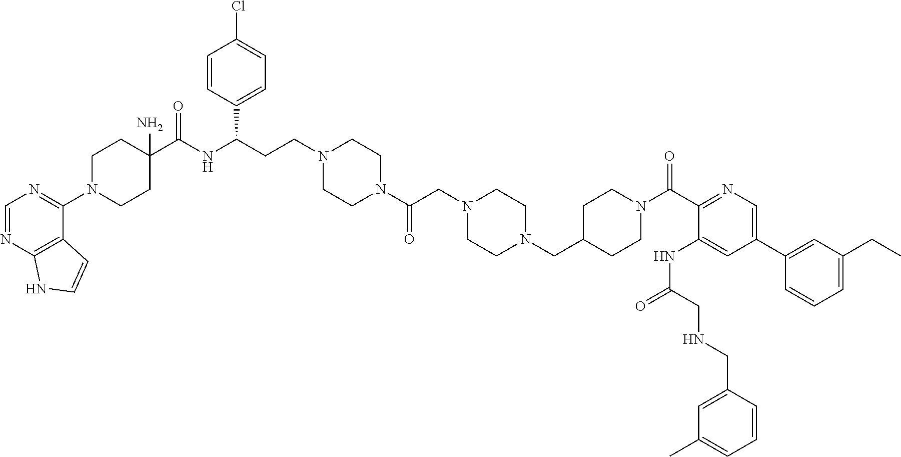 [SMILES: CCc1cccc(-c2cnc(C(=O)N3CCC(CN4CCN(CC(=O)N5CCN(CC[C@H](NC(=O)C6(N)CCN(c7ncnc8[nH]ccc78)CC6)c6ccc(Cl)cc6)CC5)CC4)CC3)c(NC(=O)CNCc3cccc(C)c3)c2)c1] |
| I-36 | 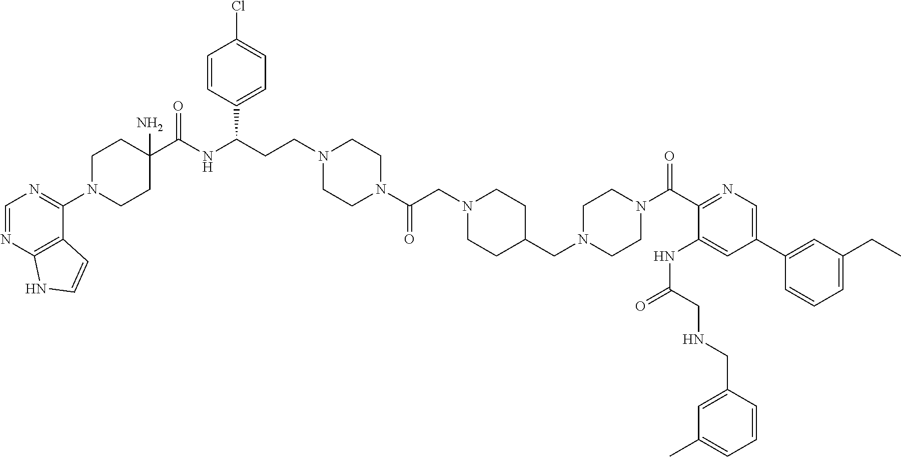 [SMILES: CCc1cccc(-c2cnc(C(=O)N3CCN(CC4CCN(CC(=O)N5CCN(CC[C@H](NC(=O)C6(N)CCN(c7ncnc8[nH]ccc78)CC6)c6ccc(Cl)cc6)CC5)CC4)CC3)c(NC(=O)CNCc3cccc(C)c3)c2)c1] |
| I-37 | 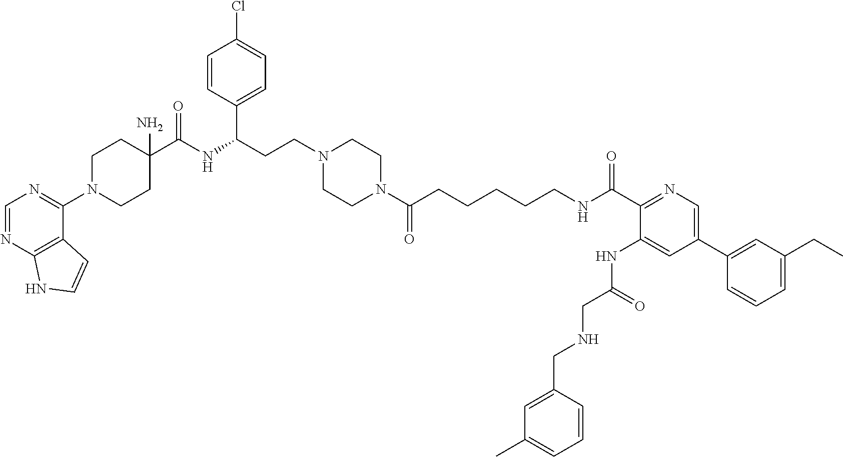 [SMILES: CCc1cccc(-c2cnc(C(=O)NCCCCCC(=O)N3CCN(CC[C@H](NC(=O)C4(N)CCN(c5ncnc6[nH]ccc56)CC4)c4ccc(Cl)cc4)CC3)c(NC(=O)CNCc3cccc(C)c3)c2)c1] |
| I-38 | 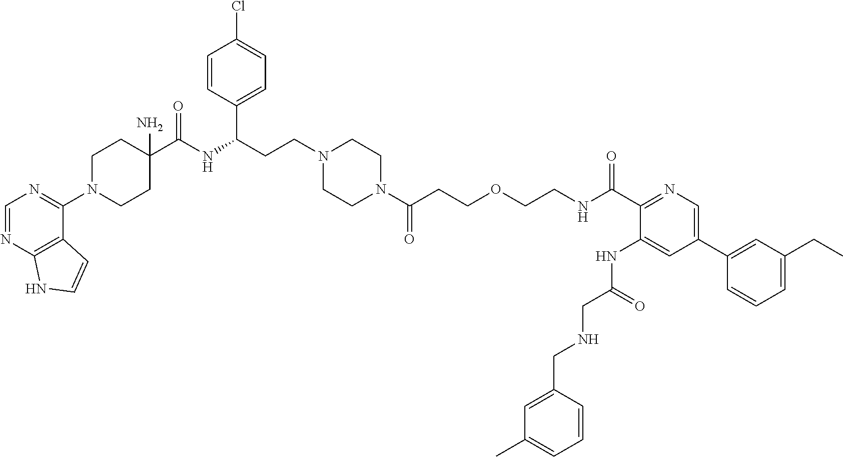 [SMILES: CCc1cccc(-c2cnc(C(=O)NCCOCCC(=O)N3CCN(CC[C@H](NC(=O)C4(N)CCN(c5ncnc6[nH]ccc56)CC4)c4ccc(Cl)cc4)CC3)c(NC(=O)CNCc3cccc(C)c3)c2)c1] |
| I-39 | 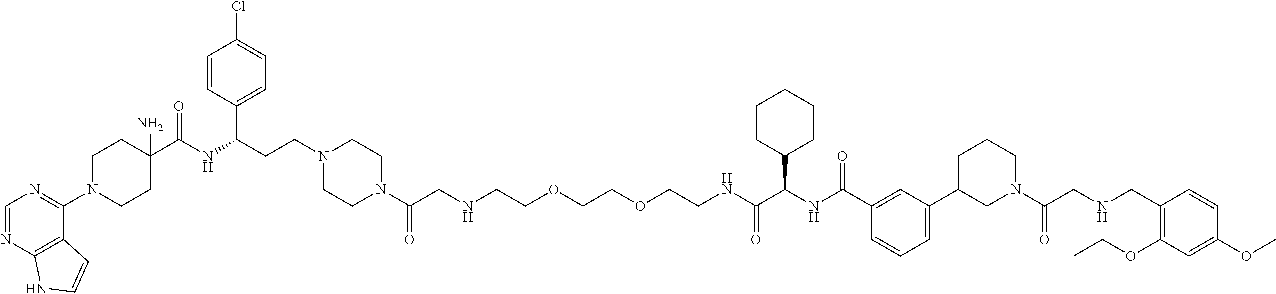 [SMILES: CCOc1cc(OC)ccc1CNCC(=O)N1CCCC(c2cccc(C(=O)N[C@@H](C(=O)NCCOCCOCCNCC(=O)N3CCN(CC[C@H](NC(=O)C4(N)CCN(c5ncnc6[nH]ccc56)CC4)c4ccc(Cl)cc4)CC3)C3CCCCC3)c2)C1] |
| I-40 | 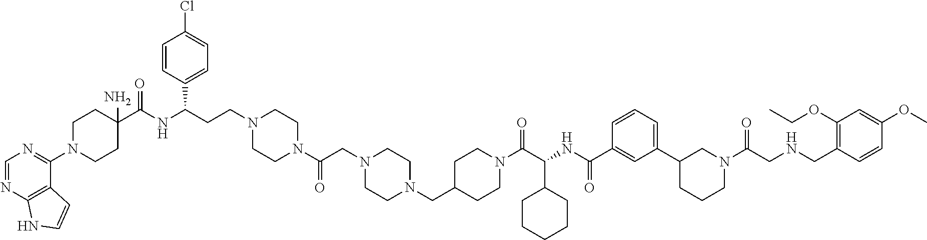 [SMILES: CCOc1cc(OC)ccc1CNCC(=O)N1CCCC(c2cccc(C(=O)N[C@@H](C(=O)N3CCC(CN4CCN(CC(=O)N5CCN(CC[C@H](NC(=O)C6(N)CCN(c7ncnc8[nH]ccc78)CC6)c6ccc(Cl)cc6)CC5)CC4)CC3)C3CCCCC3)c2)C1] |
| I-41 | 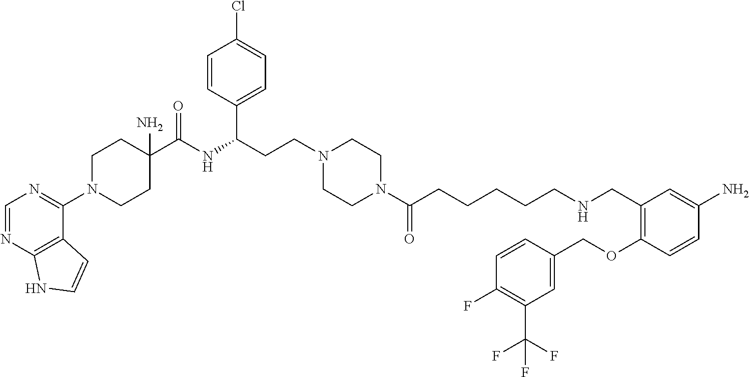 [SMILES: Nc1ccc(OCc2ccc(F)c(C(F)(F)F)c2)c(CNCCCCCC(=O)N2CCN(CC[C@H](NC(=O)C3(N)CCN(c4ncnc5[nH]ccc45)CC3)c3ccc(Cl)cc3)CC2)c1] |
| I-42 | 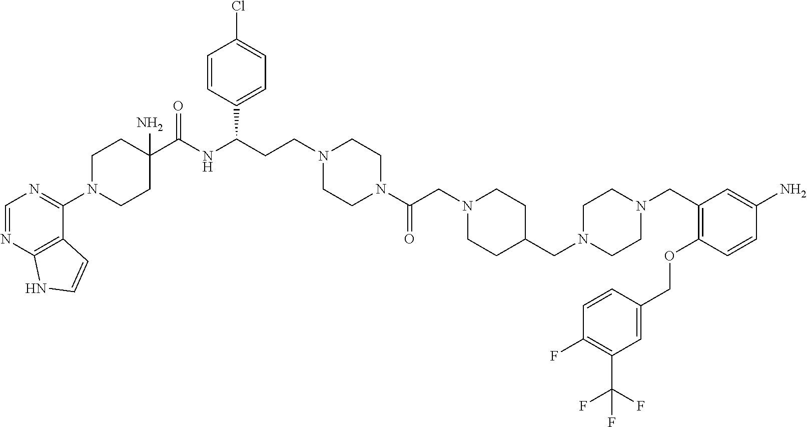 [SMILES: Nc1ccc(OCc2ccc(F)c(C(F)(F)F)c2)c(CN2CCN(CC3CCN(CC(=O)N4CCN(CC[C@H](NC(=O)C5(N)CCN(c6ncnc7[nH]ccc67)CC5)c5ccc(Cl)cc5)CC4)CC3)CC2)c1] |
| I-43 | 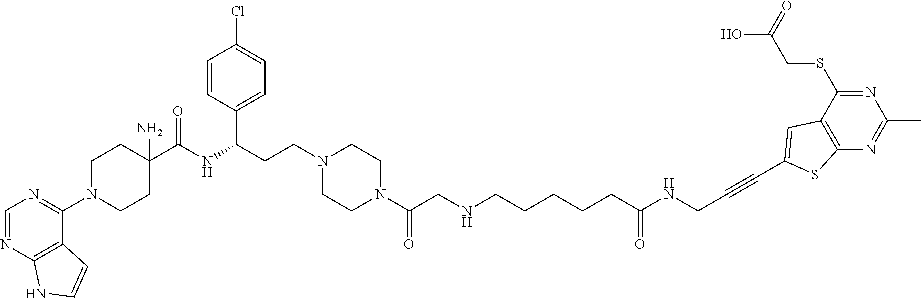 [SMILES: Cc1nc(SCC(=O)O)c2cc(C#CCNC(=O)CCCCCNCC(=O)N3CCN(CC[C@H](NC(=O)C4(N)CCN(c5ncnc6[nH]ccc56)CC4)c4ccc(Cl)cc4)CC3)sc2n1] |
| I-44 | 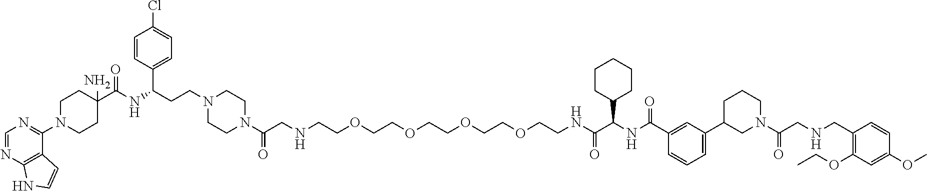 [SMILES: CCOc1cc(OC)ccc1CNCC(=O)N1CCCC(c2cccc(C(=O)N[C@@H](C(=O)NCCOCCOCCOCCOCCNCC(=O)N3CCN(CC[C@H](NC(=O)C4(N)CCN(c5ncnc6[nH]ccc56)CC4)c4ccc(Cl)cc4)CC3)C3CCCCC3)c2)C1] |
| I-45 | 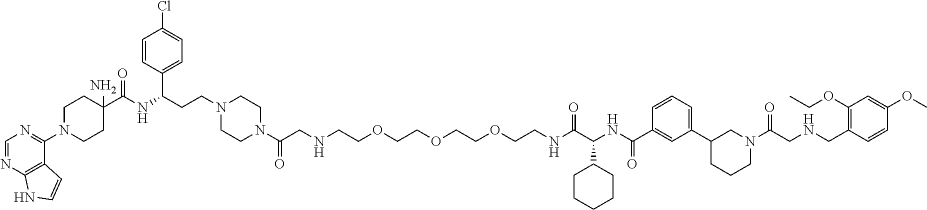 [SMILES: CCOc1cc(OC)ccc1CNCC(=O)N1CCCC(c2cccc(C(=O)N[C@@H](C(=O)NCCOCCOCCOCCNCC(=O)N3CCN(CC[C@H](NC(=O)C4(N)CCN(c5ncnc6[nH]ccc56)CC4)c4ccc(Cl)cc4)CC3)C3CCCCC3)c2)C1] |
| I-46 | 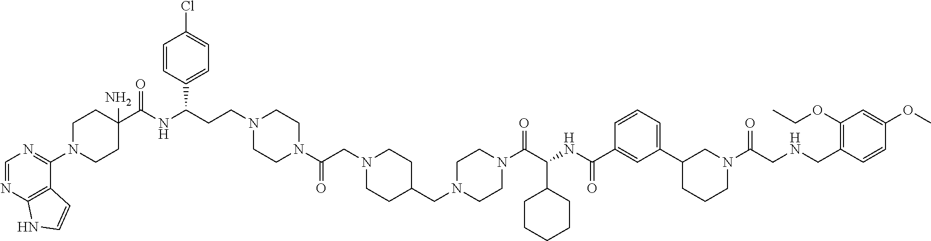 [SMILES: CCOc1cc(OC)ccc1CNCC(=O)N1CCCC(c2cccc(C(=O)N[C@@H](C(=O)N3CCN(CC4CCN(CC(=O)N5CCN(CC[C@H](NC(=O)C6(N)CCN(c7ncnc8[nH]ccc78)CC6)c6ccc(Cl)cc6)CC5)CC4)CC3)C3CCCCC3)c2)C1] |
| I-47 | 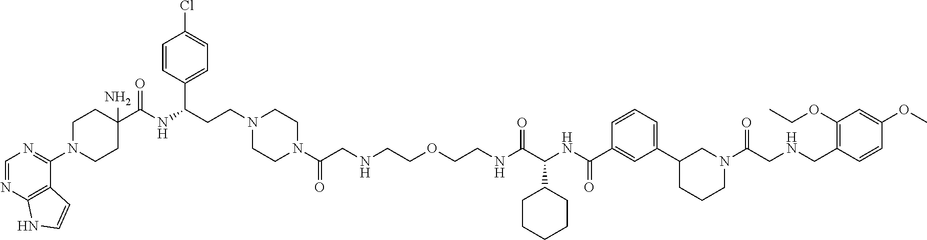 [SMILES: CCOc1cc(OC)ccc1CNCC(=O)N1CCCC(c2cccc(C(=O)N[C@@H](C(=O)NCCOCCNCC(=O)N3CCN(CC[C@H](NC(=O)C4(N)CCN(c5ncnc6[nH]ccc56)CC4)c4ccc(Cl)cc4)CC3)C3CCCCC3)c2)C1] |
| I-48 | 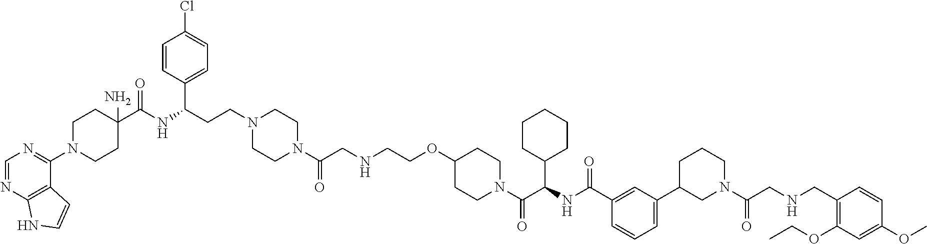 [SMILES: CCOc1cc(OC)ccc1CNCC(=O)N1CCCC(c2cccc(C(=O)N[C@@H](C(=O)N3CCC(OCCNCC(=O)N4CCN(CC[C@H](NC(=O)C5(N)CCN(c6ncnc7[nH]ccc67)CC5)c5ccc(Cl)cc5)CC4)CC3)C3CCCCC3)c2)C1] |
| I-49 | 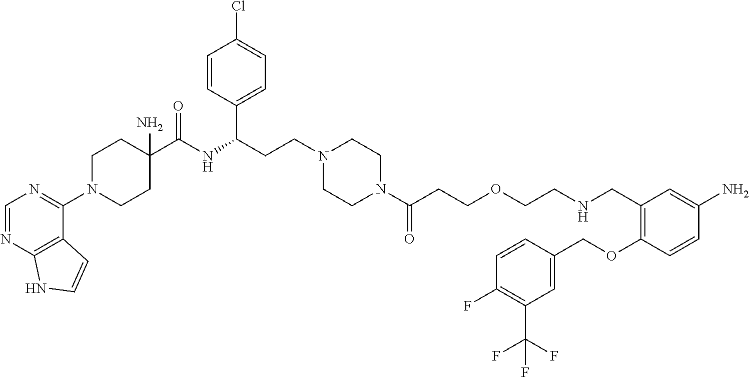 [SMILES: Nc1ccc(OCc2ccc(F)c(C(F)(F)F)c2)c(CNCCOCCC(=O)N2CCN(CC[C@H](NC(=O)C3(N)CCN(c4ncnc5[nH]ccc45)CC3)c3ccc(Cl)cc3)CC2)c1] |
| I-50 | 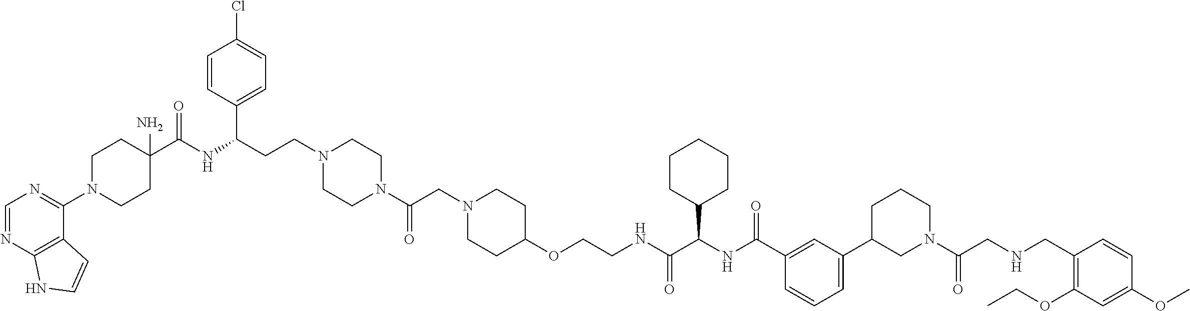 [SMILES: CCOc1cc(OC)ccc1CNCC(=O)N1CCCC(c2cccc(C(=O)N[C@@H](C(=O)NCCOC3CCN(CC(=O)N4CCN(CC[C@H](NC(=O)C5(N)CCN(c6ncnc7[nH]ccc67)CC5)c5ccc(Cl)cc5)CC4)CC3)C3CCCCC3)c2)C1] |
| I-51 | 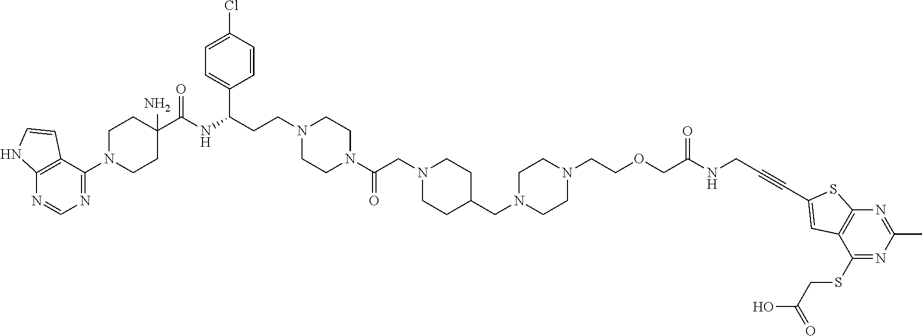 [SMILES: Cc1nc(SCC(=O)O)c2cc(C#CCNC(=O)COCCN3CCN(CC4CCN(CC(=O)N5CCN(CC[C@H](NC(=O)C6(N)CCN(c7ncnc8[nH]ccc78)CC6)c6ccc(Cl)cc6)CC5)CC4)CC3)sc2n1] |
| I-52 | 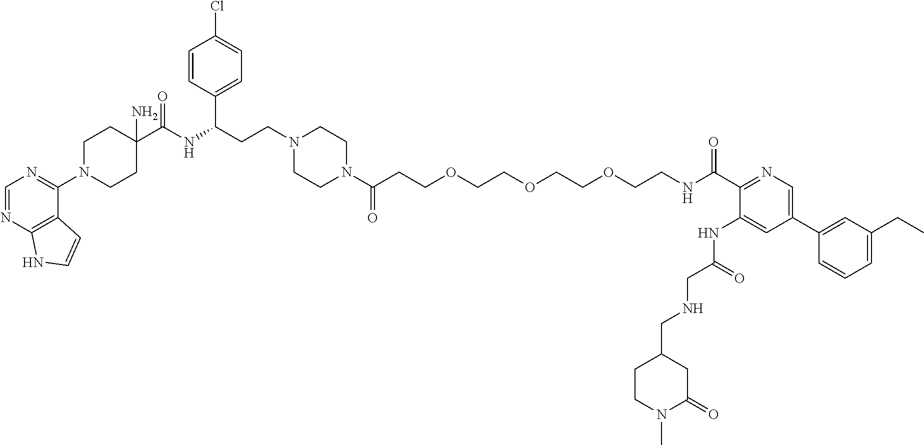 [SMILES: CCc1cccc(-c2cnc(C(=O)NCCOCCOCCOCCC(=O)N3CCN(CC[C@H](NC(=O)C4(N)CCN(c5ncnc6[nH]ccc56)CC4)c4ccc(Cl)cc4)CC3)c(NC(=O)CNCC3CCN(C)C(=O)C3)c2)c1] |
| I-53 | 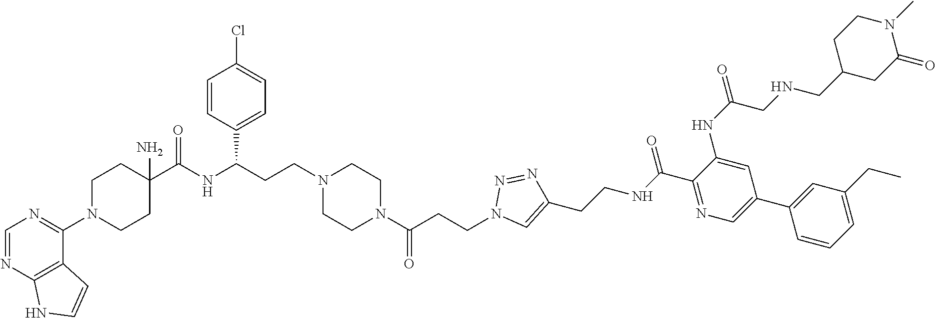 [SMILES: CCc1cccc(-c2cnc(C(=O)NCCc3cn(CCC(=O)N4CCN(CC[C@H](NC(=O)C5(N)CCN(c6ncnc7[nH]ccc67)CC5)c5ccc(Cl)cc5)CC4)nn3)c(NC(=O)CNCC3CCN(C)C(=O)C3)c2)c1] |
| I-54 | 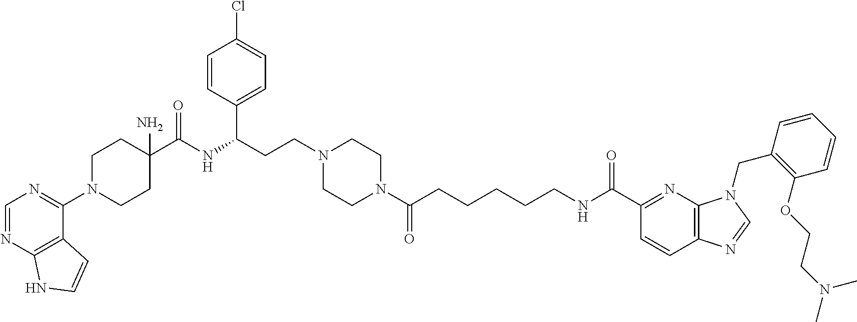 [SMILES: CN(C)CCOc1ccccc1Cn1cnc2ccc(C(=O)NCCCCCC(=O)N3CCN(CC[C@H](NC(=O)C4(N)CCN(c5ncnc6[nH]ccc56)CC4)c4ccc(Cl)cc4)CC3)nc21] |
| I-55 | 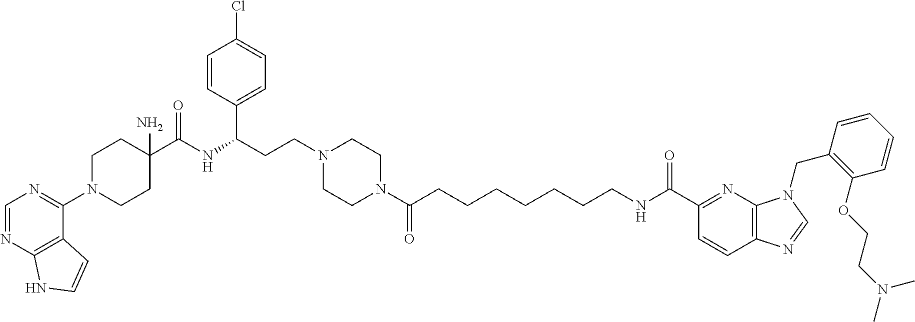 [SMILES: CN(C)CCOc1ccccc1Cn1cnc2ccc(C(=O)NCCCCCCCC(=O)N3CCN(CC[C@H](NC(=O)C4(N)CCN(c5ncnc6[nH]ccc56)CC4)c4ccc(Cl)cc4)CC3)nc21] |
| I-56 | 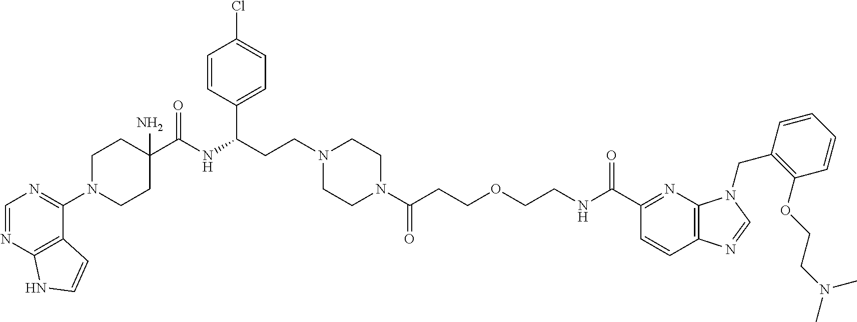 [SMILES: CN(C)CCOc1ccccc1Cn1cnc2ccc(C(=O)NCCOCCC(=O)N3CCN(CC[C@H](NC(=O)C4(N)CCN(c5ncnc6[nH]ccc56)CC4)c4ccc(Cl)cc4)CC3)nc21] |
| I-57 | 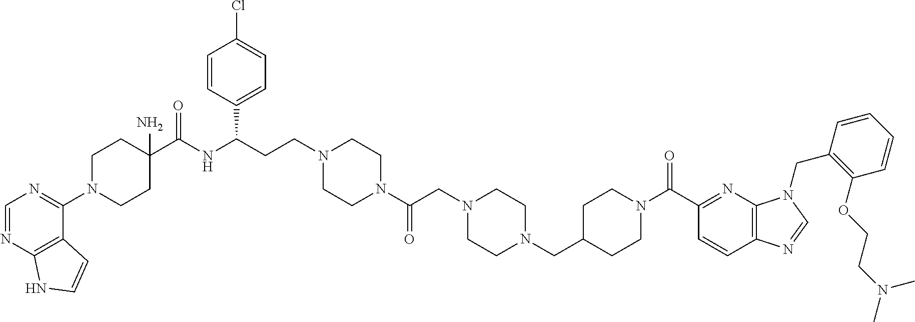 [SMILES: CN(C)CCOc1ccccc1Cn1cnc2ccc(C(=O)N3CCC(CN4CCN(CC(=O)N5CCN(CC[C@H](NC(=O)C6(N)CCN(c7ncnc8[nH]ccc78)CC6)c6ccc(Cl)cc6)CC5)CC4)CC3)nc21] |
| I-65 | 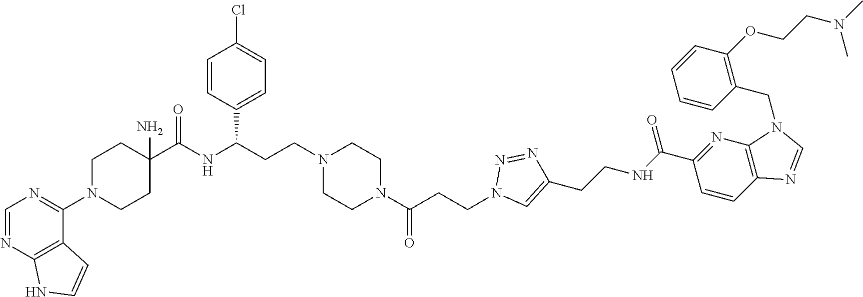 [SMILES: CN(C)CCOc1ccccc1Cn1cnc2ccc(C(=O)NCCc3cn(CCC(=O)N4CCN(CC[C@H](NC(=O)C5(N)CCN(c6ncnc7[nH]ccc67)CC5)c5ccc(Cl)cc5)CC4)nn3)nc21] |
| I-66 | 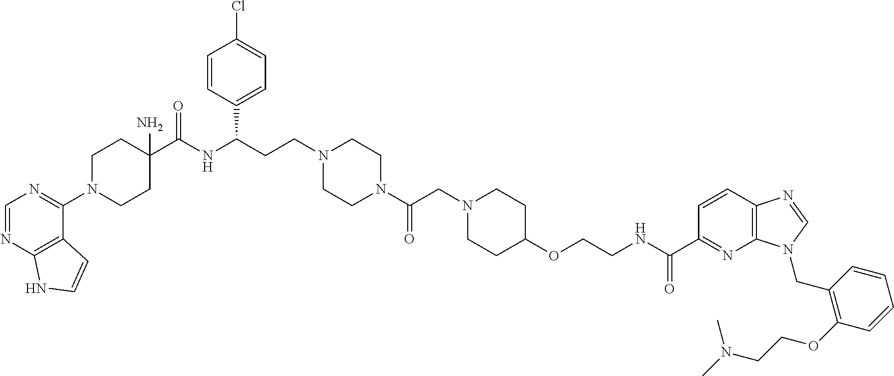 [SMILES: CN(C)CCOc1ccccc1Cn1cnc2ccc(C(=O)NCCOC3CCN(CC(=O)N4CCN(CC[C@H](NC(=O)C5(N)CCN(c6ncnc7[nH]ccc67)CC5)c5ccc(Cl)cc5)CC4)CC3)nc21] |
| I-67 | 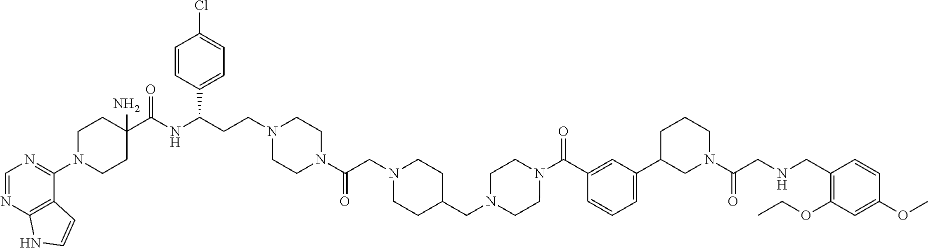 [SMILES: CCOc1cc(OC)ccc1CNCC(=O)N1CCCC(c2cccc(C(=O)N3CCN(CC4CCN(CC(=O)N5CCN(CC[C@H](NC(=O)C6(N)CCN(c7ncnc8[nH]ccc78)CC6)c6ccc(Cl)cc6)CC5)CC4)CC3)c2)C1] |
| I-68 | 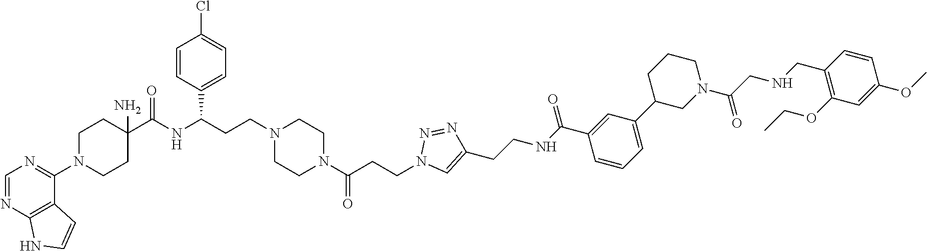 [SMILES: CCOc1cc(OC)ccc1CNCC(=O)N1CCCC(c2cccc(C(=O)NCCc3cn(CCC(=O)N4CCN(CC[C@H](NC(=O)C5(N)CCN(c6ncnc7[nH]ccc67)CC5)c5ccc(Cl)cc5)CC4)nn3)c2)C1] |
| I-69 | 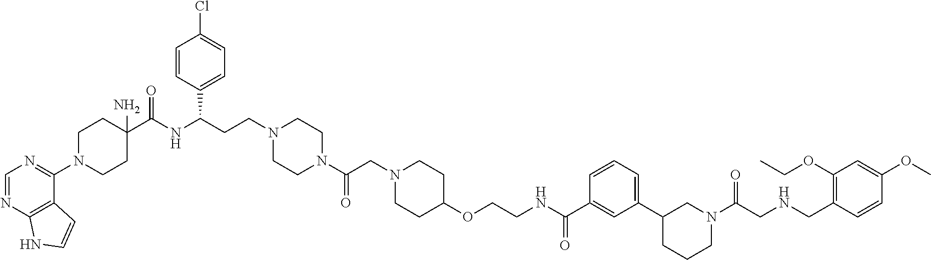 [SMILES: CCOc1cc(OC)ccc1CNCC(=O)N1CCCC(c2cccc(C(=O)NCCOC3CCN(CC(=O)N4CCN(CC[C@H](NC(=O)C5(N)CCN(c6ncnc7[nH]ccc67)CC5)c5ccc(Cl)cc5)CC4)CC3)c2)C1] |
| I-73 | 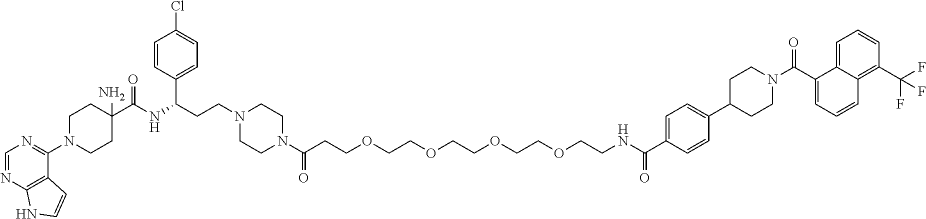 [SMILES: NC1(C(=O)N[C@@H](CCN2CCN(C(=O)CCOCCOCCOCCOCCNC(=O)c3ccc(C4CCN(C(=O)c5cccc6c(C(F)(F)F)cccc56)CC4)cc3)CC2)c2ccc(Cl)cc2)CCN(c2ncnc3[nH]ccc23)CC1] |
| I-74 | 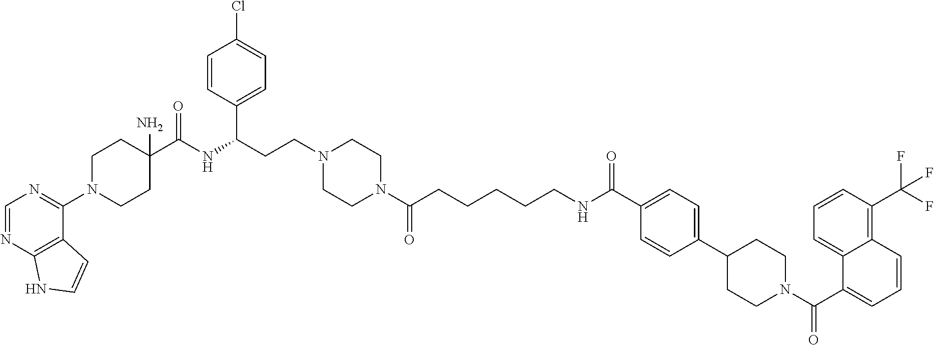 [SMILES: NC1(C(=O)N[C@@H](CCN2CCN(C(=O)CCCCCNC(=O)c3ccc(C4CCN(C(=O)c5cccc6c(C(F)(F)F)cccc56)CC4)cc3)CC2)c2ccc(Cl)cc2)CCN(c2ncnc3[nH]ccc23)CC1] |
| I-75 | 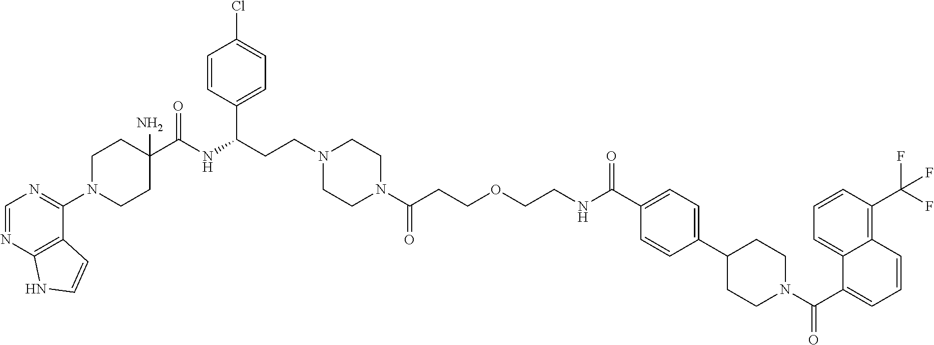 [SMILES: NC1(C(=O)N[C@@H](CCN2CCN(C(=O)CCOCCNC(=O)c3ccc(C4CCN(C(=O)c5cccc6c(C(F)(F)F)cccc56)CC4)cc3)CC2)c2ccc(Cl)cc2)CCN(c2ncnc3[nH]ccc23)CC1] |
| I-76 | 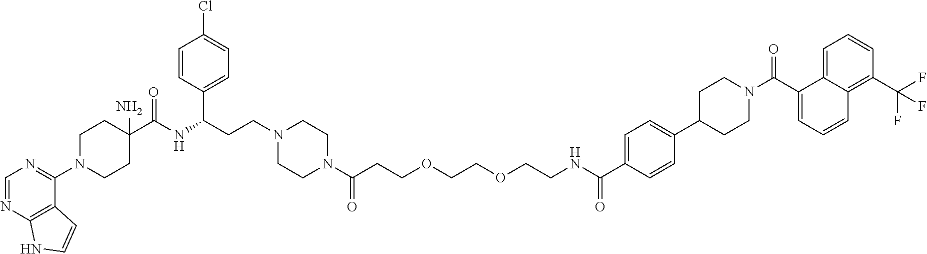 [SMILES: NC1(C(=O)N[C@@H](CCN2CCN(C(=O)CCOCCOCCNC(=O)c3ccc(C4CCN(C(=O)c5cccc6c(C(F)(F)F)cccc56)CC4)cc3)CC2)c2ccc(Cl)cc2)CCN(c2ncnc3[nH]ccc23)CC1] |
| I-77 | 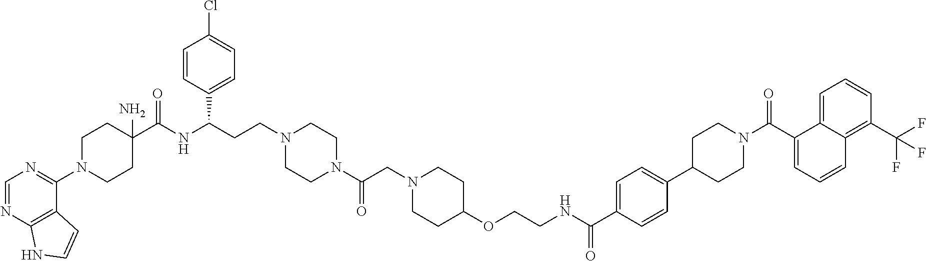 [SMILES: NC1(C(=O)N[C@@H](CCN2CCN(C(=O)CN3CCC(OCCNC(=O)c4ccc(C5CCN(C(=O)c6cccc7c(C(F)(F)F)cccc67)CC5)cc4)CC3)CC2)c2ccc(Cl)cc2)CCN(c2ncnc3[nH]ccc23)CC1] |
| I-78 | 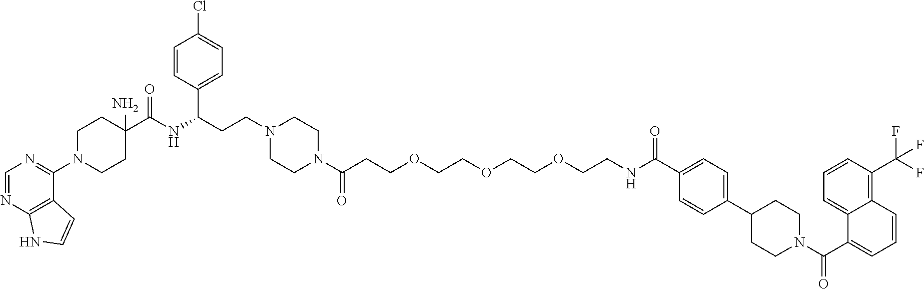 [SMILES: NC1(C(=O)N[C@@H](CCN2CCN(C(=O)CCOCCOCCOCCNC(=O)c3ccc(C4CCN(C(=O)c5cccc6c(C(F)(F)F)cccc56)CC4)cc3)CC2)c2ccc(Cl)cc2)CCN(c2ncnc3[nH]ccc23)CC1] |
| I-79 | 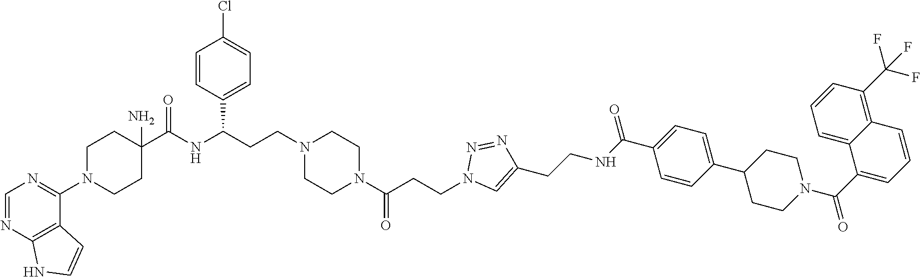 [SMILES: NC1(C(=O)N[C@@H](CCN2CCN(C(=O)CCn3cc(CCNC(=O)c4ccc(C5CCN(C(=O)c6cccc7c(C(F)(F)F)cccc67)CC5)cc4)nn3)CC2)c2ccc(Cl)cc2)CCN(c2ncnc3[nH]ccc23)CC1] |
| I-80 | 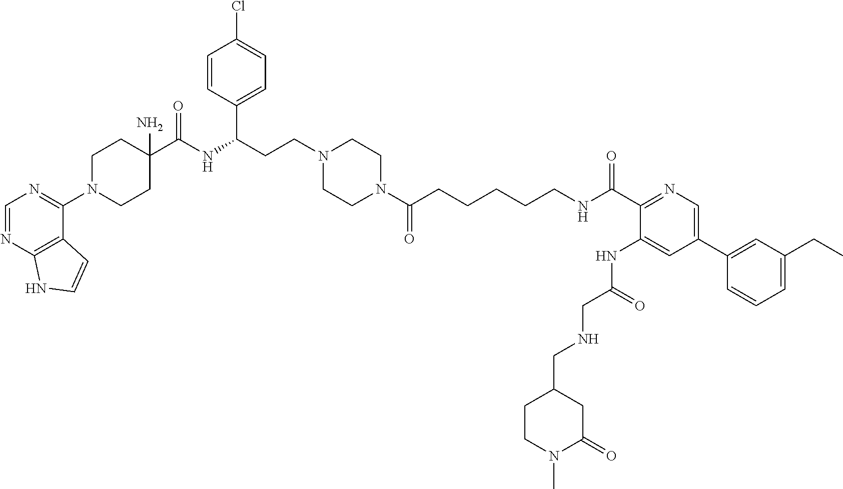 [SMILES: CCc1cccc(-c2cnc(C(=O)NCCCCCC(=O)N3CCN(CC[C@H](NC(=O)C4(N)CCN(c5ncnc6[nH]ccc56)CC4)c4ccc(Cl)cc4)CC3)c(NC(=O)CNCC3CCN(C)C(=O)C3)c2)c1] |
| I-81 | 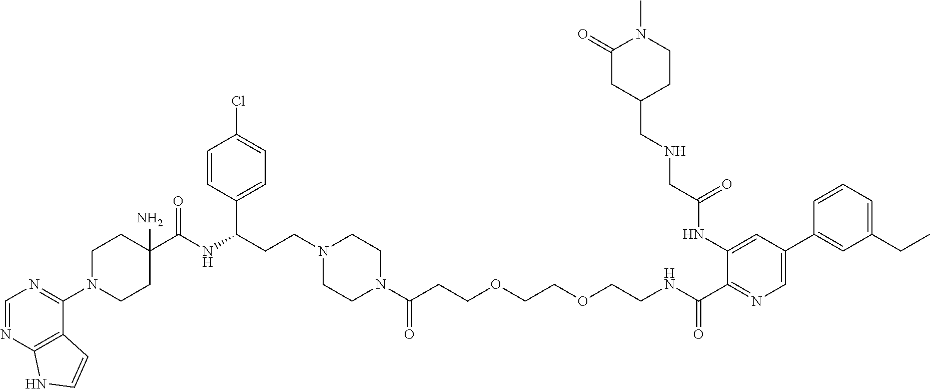 [SMILES: CCc1cccc(-c2cnc(C(=O)NCCOCCOCCC(=O)N3CCN(CC[C@H](NC(=O)C4(N)CCN(c5ncnc6[nH]ccc56)CC4)c4ccc(Cl)cc4)CC3)c(NC(=O)CNCC3CCN(C)C(=O)C3)c2)c1] |
| I-82 | 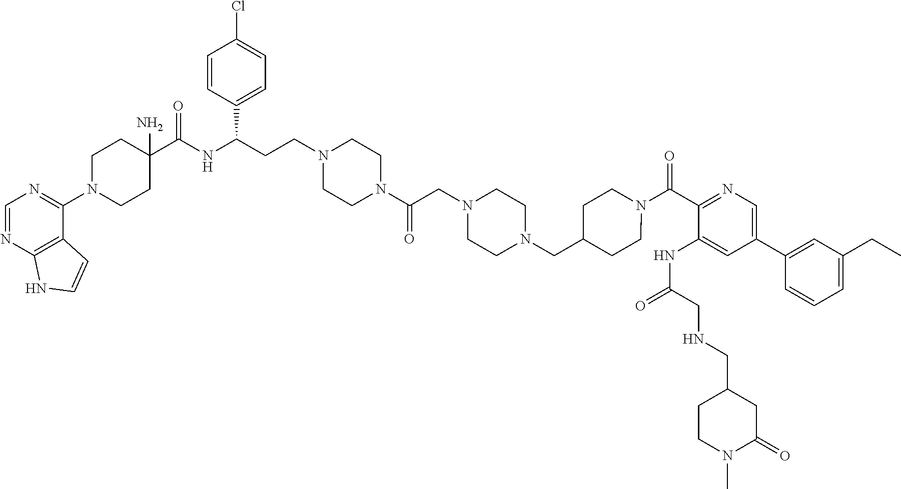 [SMILES: CCc1cccc(-c2cnc(C(=O)N3CCC(CN4CCN(CC(=O)N5CCN(CC[C@H](NC(=O)C6(N)CCN(c7ncnc8[nH]ccc78)CC6)c6ccc(Cl)cc6)CC5)CC4)CC3)c(NC(=O)CNCC3CCN(C)C(=O)C3)c2)c1] |
| I-83 | 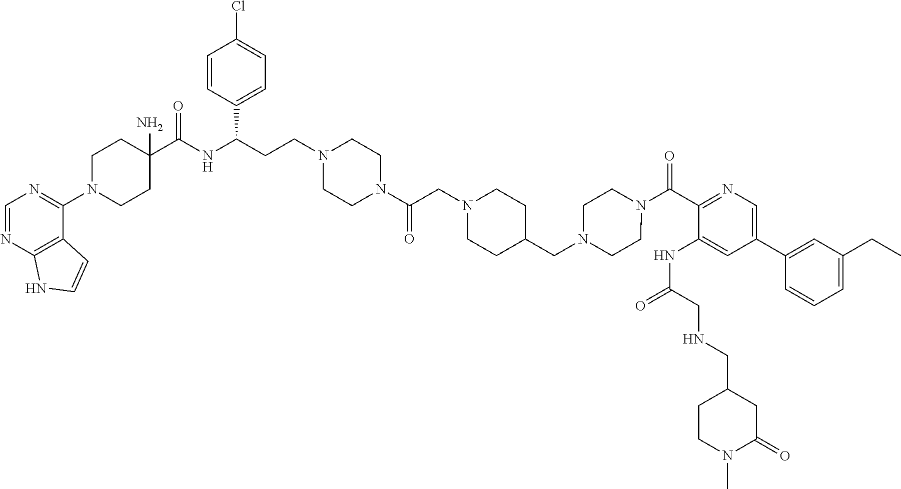 [SMILES: CCc1cccc(-c2cnc(C(=O)N3CCN(CC4CCN(CC(=O)N5CCN(CC[C@H](NC(=O)C6(N)CCN(c7ncnc8[nH]ccc78)CC6)c6ccc(Cl)cc6)CC5)CC4)CC3)c(NC(=O)CNCC3CCN(C)C(=O)C3)c2)c1] |
| I-84 | 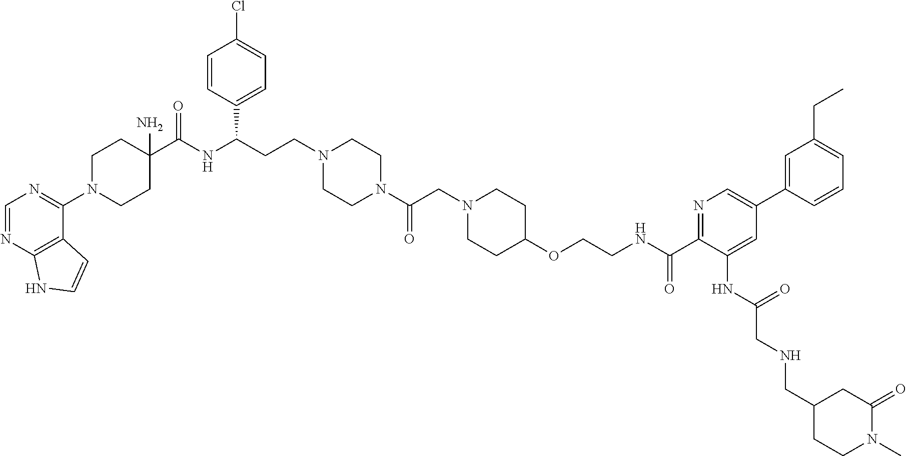 [SMILES: CCc1cccc(-c2cnc(C(=O)NCCOC3CCN(CC(=O)N4CCN(CC[C@H](NC(=O)C5(N)CCN(c6ncnc7[nH]ccc67)CC5)c5ccc(Cl)cc5)CC4)CC3)c(NC(=O)CNCC3CCN(C)C(=O)C3)c2)c1] |
| I-85 | 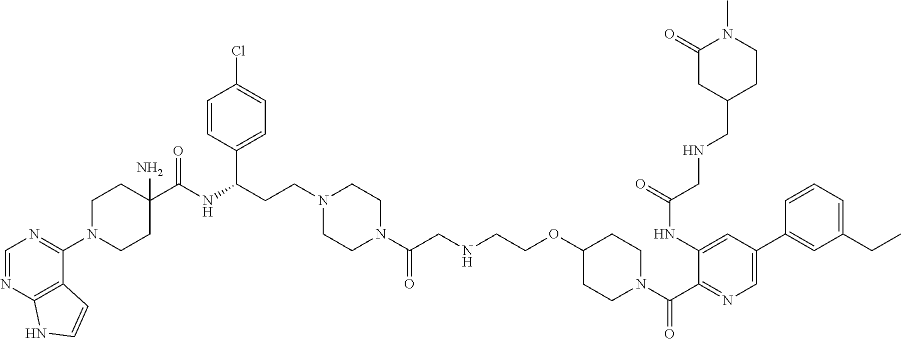 [SMILES: CCc1cccc(-c2cnc(C(=O)N3CCC(OCCNCC(=O)N4CCN(CC[C@H](NC(=O)C5(N)CCN(c6ncnc7[nH]ccc67)CC5)c5ccc(Cl)cc5)CC4)CC3)c(NC(=O)CNCC3CCN(C)C(=O)C3)c2)c1] |
| I-86 | 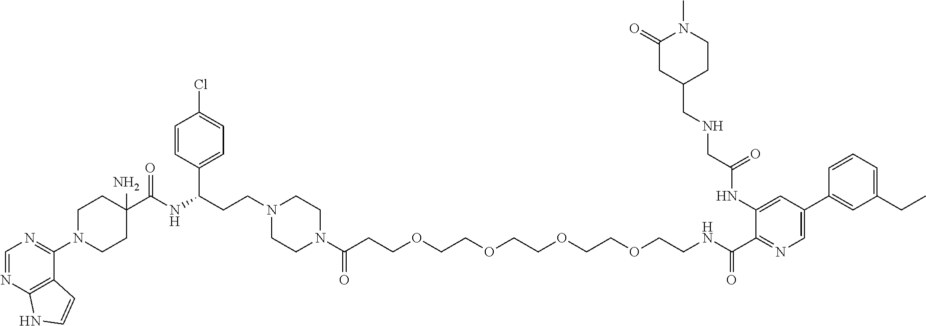 [SMILES: CCc1cccc(-c2cnc(C(=O)NCCOCCOCCOCCOCCC(=O)N3CCN(CC[C@H](NC(=O)C4(N)CCN(c5ncnc6[nH]ccc56)CC4)c4ccc(Cl)cc4)CC3)c(NC(=O)CNCC3CCN(C)C(=O)C3)c2)c1] |
| I-87 | 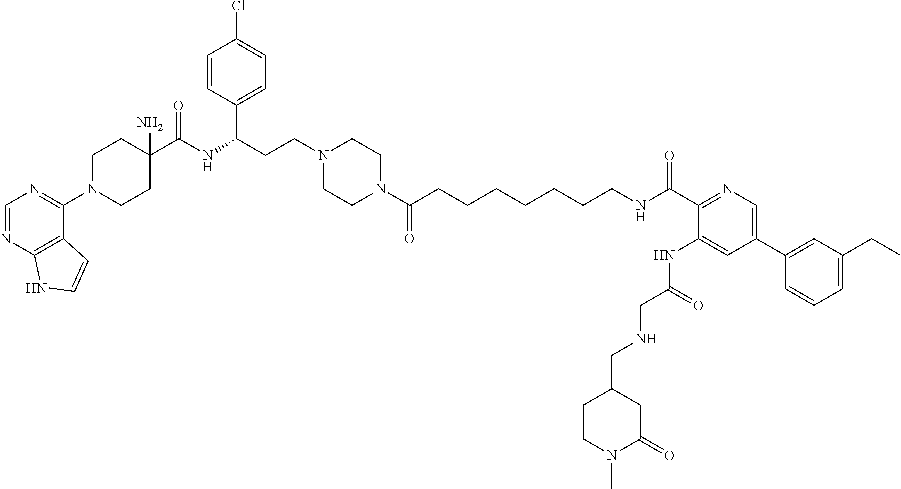 [SMILES: CCc1cccc(-c2cnc(C(=O)NCCCCCCCC(=O)N3CCN(CC[C@H](NC(=O)C4(N)CCN(c5ncnc6[nH]ccc56)CC4)c4ccc(Cl)cc4)CC3)c(NC(=O)CNCC3CCN(C)C(=O)C3)c2)c1] |
| I-88 | 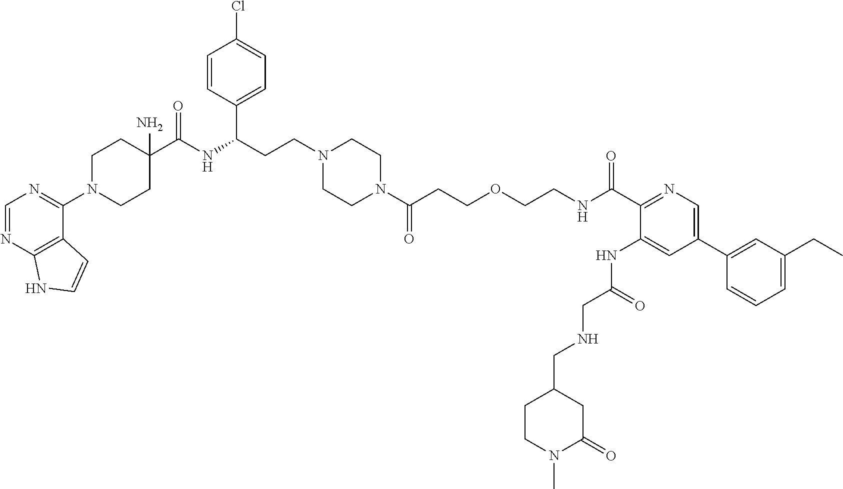 [SMILES: CCc1cccc(-c2cnc(C(=O)NCCOCCC(=O)N3CCN(CC[C@H](NC(=O)C4(N)CCN(c5ncnc6[nH]ccc56)CC4)c4ccc(Cl)cc4)CC3)c(NC(=O)CNCC3CCN(C)C(=O)C3)c2)c1] |
| I-89 | 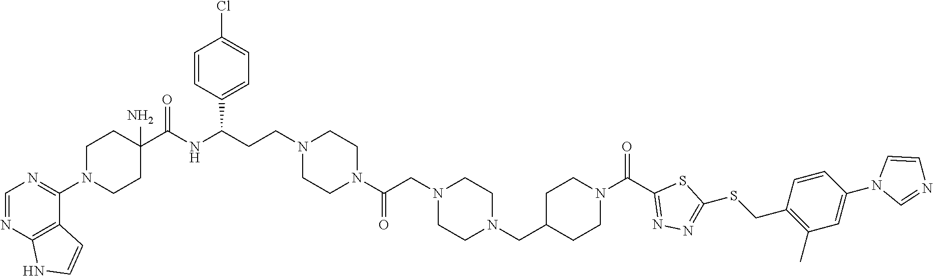 [SMILES: Cc1cc(-n2ccnc2)ccc1CSc1nnc(C(=O)N2CCC(CN3CCN(CC(=O)N4CCN(CC[C@H](NC(=O)C5(N)CCN(c6ncnc7[nH]ccc67)CC5)c5ccc(Cl)cc5)CC4)CC3)CC2)s1] |
| I-90 | 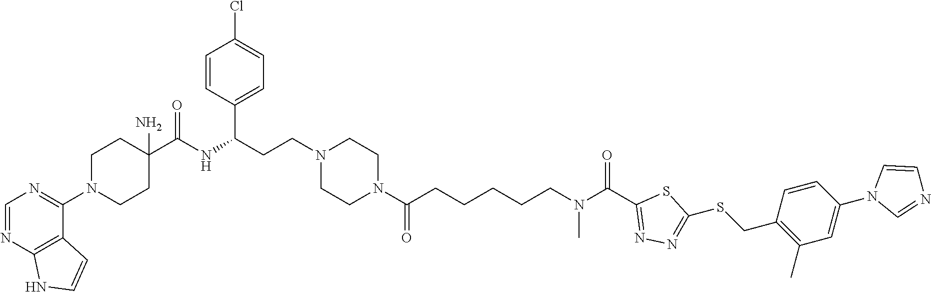 [SMILES: Cc1cc(-n2ccnc2)ccc1CSc1nnc(C(=O)N(C)CCCCCC(=O)N2CCN(CC[C@H](NC(=O)C3(N)CCN(c4ncnc5[nH]ccc45)CC3)c3ccc(Cl)cc3)CC2)s1] |
| I-91 | 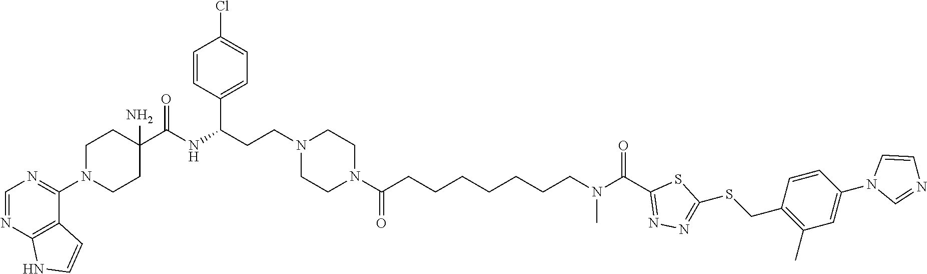 [SMILES: Cc1cc(-n2ccnc2)ccc1CSc1nnc(C(=O)N(C)CCCCCCCC(=O)N2CCN(CC[C@H](NC(=O)C3(N)CCN(c4ncnc5[nH]ccc45)CC3)c3ccc(Cl)cc3)CC2)s1] |
| I-92 | 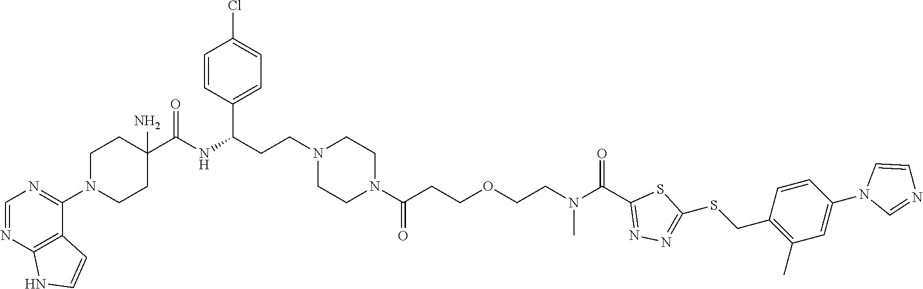 [SMILES: Cc1cc(-n2ccnc2)ccc1CSc1nnc(C(=O)N(C)CCOCCC(=O)N2CCN(CC[C@H](NC(=O)C3(N)CCN(c4ncnc5[nH]ccc45)CC3)c3ccc(Cl)cc3)CC2)s1] |
| I-93 | 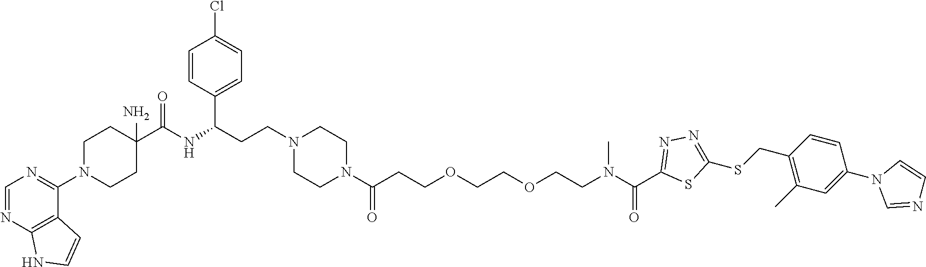 [SMILES: Cc1cc(-n2ccnc2)ccc1CSc1nnc(C(=O)N(C)CCOCCOCCC(=O)N2CCN(CC[C@H](NC(=O)C3(N)CCN(c4ncnc5[nH]ccc45)CC3)c3ccc(Cl)cc3)CC2)s1] |
| I-94 | 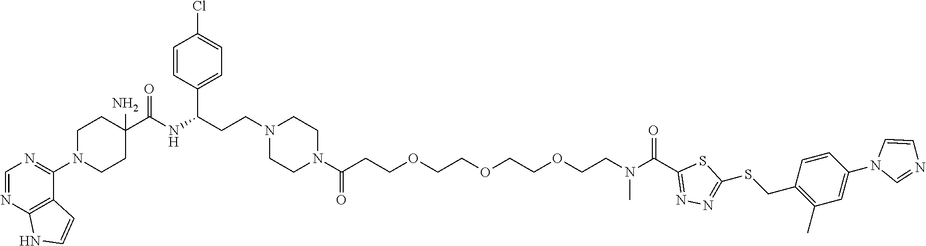 [SMILES: Cc1cc(-n2ccnc2)ccc1CSc1nnc(C(=O)N(C)CCOCCOCCOCCC(=O)N2CCN(CC[C@H](NC(=O)C3(N)CCN(c4ncnc5[nH]ccc45)CC3)c3ccc(Cl)cc3)CC2)s1] |
| I-95 | 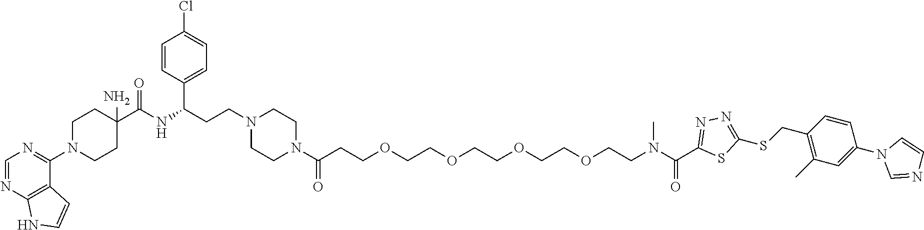 [SMILES: Cc1cc(-n2ccnc2)ccc1CSc1nnc(C(=O)N(C)CCOCCOCCOCCOCCC(=O)N2CCN(CC[C@H](NC(=O)C3(N)CCN(c4ncnc5[nH]ccc45)CC3)c3ccc(Cl)cc3)CC2)s1] |
| I-96 | 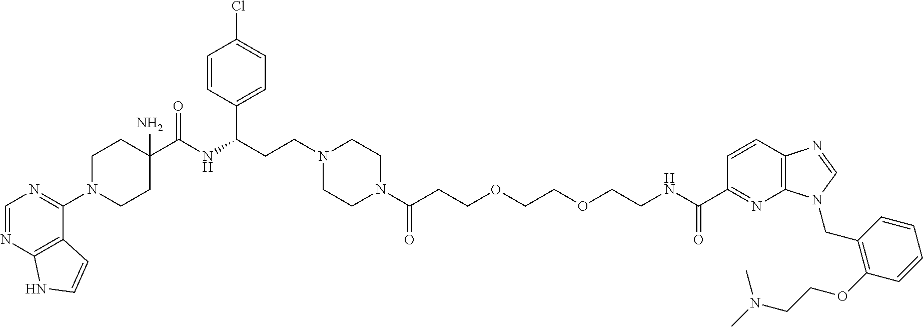 [SMILES: CN(C)CCOc1ccccc1Cn1cnc2ccc(C(=O)NCCOCCOCCC(=O)N3CCN(CC[C@H](NC(=O)C4(N)CCN(c5ncnc6[nH]ccc56)CC4)c4ccc(Cl)cc4)CC3)nc21] |
| I-97 | 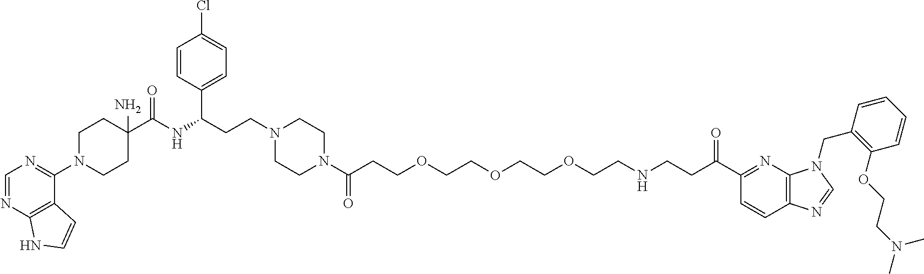 [SMILES: CN(C)CCOc1ccccc1Cn1cnc2ccc(C(=O)CCNCCOCCOCCOCCC(=O)N3CCN(CC[C@H](NC(=O)C4(N)CCN(c5ncnc6[nH]ccc56)CC4)c4ccc(Cl)cc4)CC3)nc21] |
| I-98 | 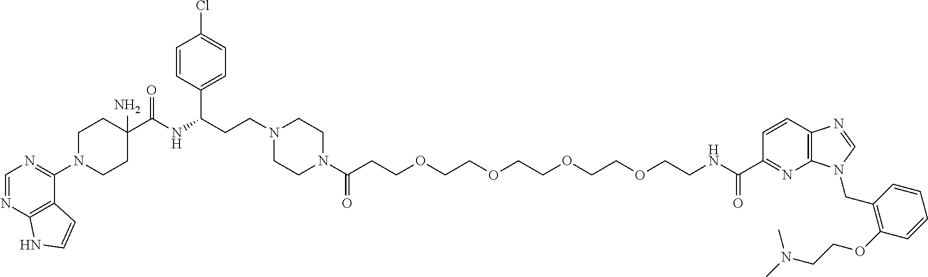 [SMILES: CN(C)CCOc1ccccc1Cn1cnc2ccc(C(=O)NCCOCCOCCOCCOCCC(=O)N3CCN(CC[C@H](NC(=O)C4(N)CCN(c5ncnc6[nH]ccc56)CC4)c4ccc(Cl)cc4)CC3)nc21] |
| I-99 | 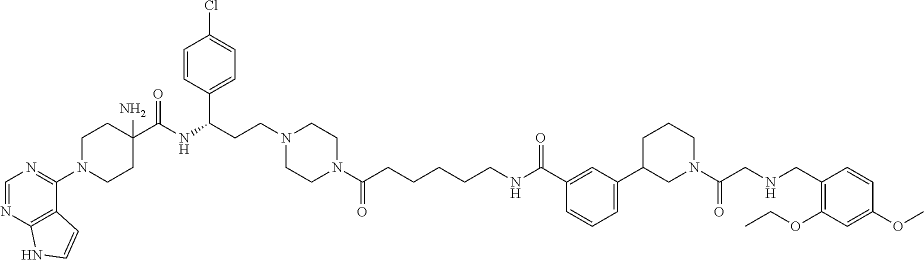 [SMILES: CCOc1cc(OC)ccc1CNCC(=O)N1CCCC(c2cccc(C(=O)NCCCCCC(=O)N3CCN(CC[C@H](NC(=O)C4(N)CCN(c5ncnc6[nH]ccc56)CC4)c4ccc(Cl)cc4)CC3)c2)C1] |
| I-100 | 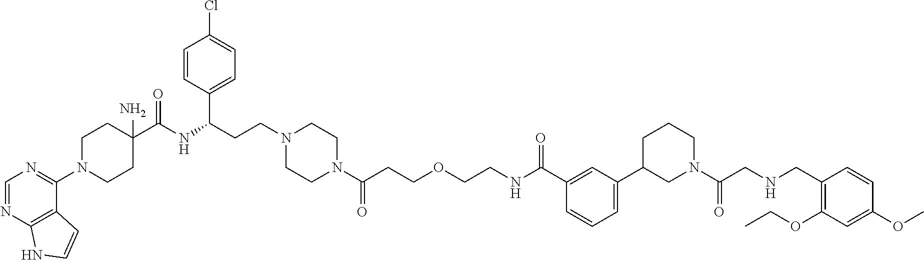 [SMILES: CCOc1cc(OC)ccc1CNCC(=O)N1CCCC(c2cccc(C(=O)NCCOCCC(=O)N3CCN(CC[C@H](NC(=O)C4(N)CCN(c5ncnc6[nH]ccc56)CC4)c4ccc(Cl)cc4)CC3)c2)C1] |
| I-101 | 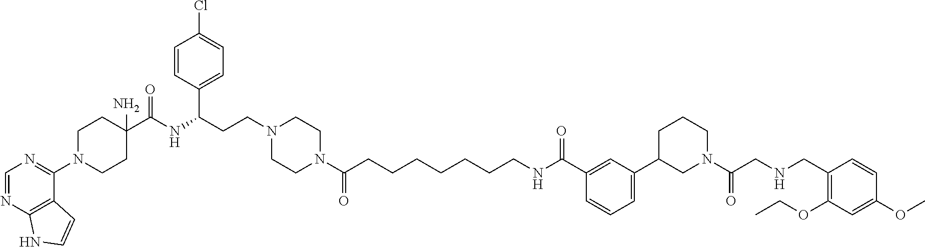 [SMILES: CCOc1cc(OC)ccc1CNCC(=O)N1CCCC(c2cccc(C(=O)NCCCCCCCC(=O)N3CCN(CC[C@H](NC(=O)C4(N)CCN(c5ncnc6[nH]ccc56)CC4)c4ccc(Cl)cc4)CC3)c2)C1] |
| I-102 | 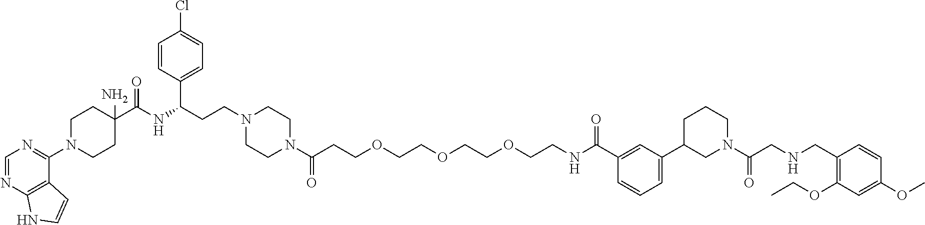 [SMILES: CCOc1cc(OC)ccc1CNCC(=O)N1CCCC(c2cccc(C(=O)NCCOCCOCCOCCC(=O)N3CCN(CC[C@H](NC(=O)C4(N)CCN(c5ncnc6[nH]ccc56)CC4)c4ccc(Cl)cc4)CC3)c2)C1] |
| I-103 | 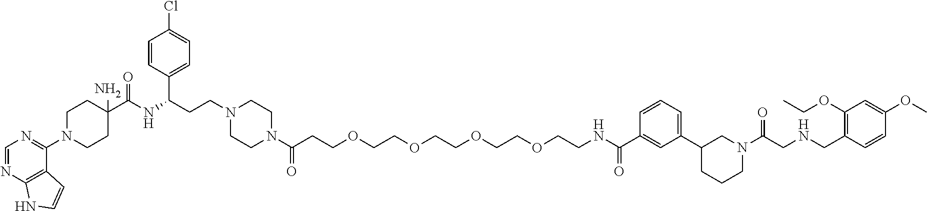 [SMILES: CCOc1cc(OC)ccc1CNCC(=O)N1CCCC(c2cccc(C(=O)NCCOCCOCCOCCOCCC(=O)N3CCN(CC[C@H](NC(=O)C4(N)CCN(c5ncnc6[nH]ccc56)CC4)c4ccc(Cl)cc4)CC3)c2)C1] |
| I-104 | 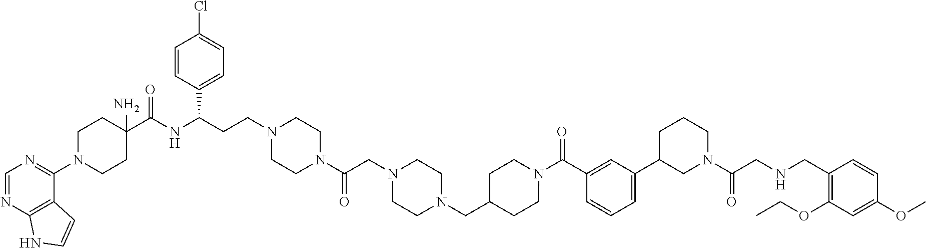 [SMILES: CCOc1cc(OC)ccc1CNCC(=O)N1CCCC(c2cccc(C(=O)N3CCC(CN4CCN(CC(=O)N5CCN(CC[C@H](NC(=O)C6(N)CCN(c7ncnc8[nH]ccc78)CC6)c6ccc(Cl)cc6)CC5)CC4)CC3)c2)C1] |
| I-105 | 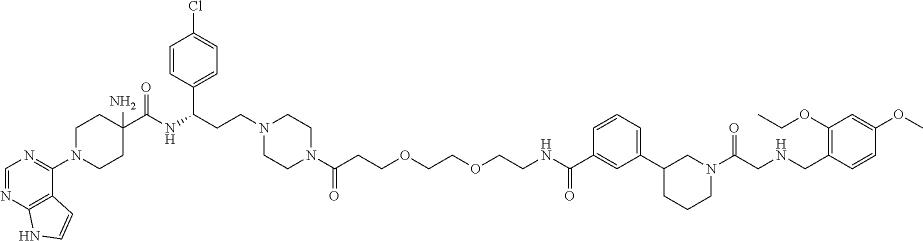 [SMILES: CCOc1cc(OC)ccc1CNCC(=O)N1CCCC(c2cccc(C(=O)NCCOCCOCCC(=O)N3CCN(CC[C@H](NC(=O)C4(N)CCN(c5ncnc6[nH]ccc56)CC4)c4ccc(Cl)cc4)CC3)c2)C1] |
| I-106 | 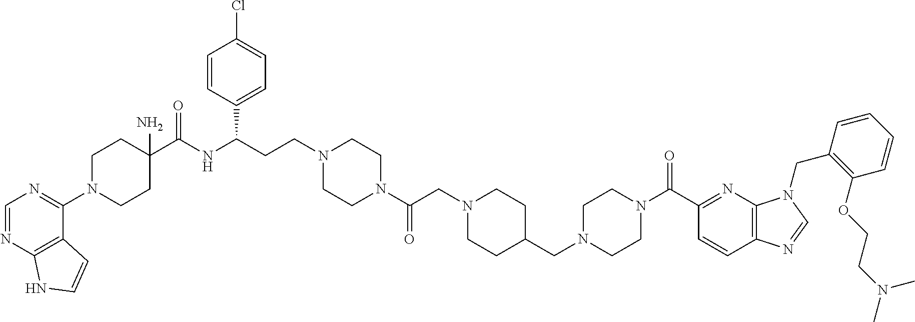 [SMILES: CN(C)CCOc1ccccc1Cn1cnc2ccc(C(=O)N3CCN(CC4CCN(CC(=O)N5CCN(CC[C@H](NC(=O)C6(N)CCN(c7ncnc8[nH]ccc78)CC6)c6ccc(Cl)cc6)CC5)CC4)CC3)nc21] |
| I-107 | 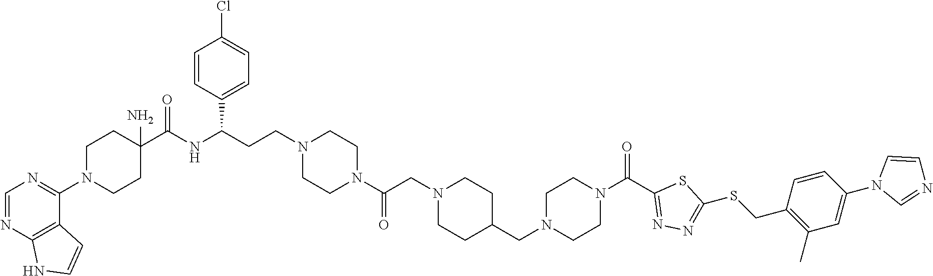 [SMILES: Cc1cc(-n2ccnc2)ccc1CSc1nnc(C(=O)N2CCN(CC3CCN(CC(=O)N4CCN(CC[C@H](NC(=O)C5(N)CCN(c6ncnc7[nH]ccc67)CC5)c5ccc(Cl)cc5)CC4)CC3)CC2)s1] |
| I-108 | 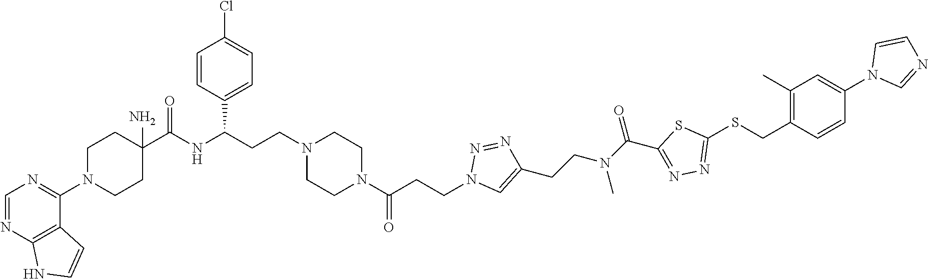 [SMILES: Cc1cc(-n2ccnc2)ccc1CSc1nnc(C(=O)N(C)CCc2cn(CCC(=O)N3CCN(CC[C@H](NC(=O)C4(N)CCN(c5ncnc6[nH]ccc56)CC4)c4ccc(Cl)cc4)CC3)nn2)s1] |
| I-109 | 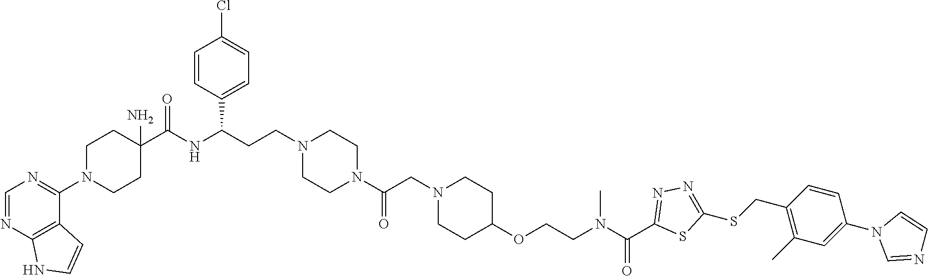 [SMILES: Cc1cc(-n2ccnc2)ccc1CSc1nnc(C(=O)N(C)CCOC2CCN(CC(=O)N3CCN(CC[C@H](NC(=O)C4(N)CCN(c5ncnc6[nH]ccc56)CC4)c4ccc(Cl)cc4)CC3)CC2)s1] |
| I-110 | 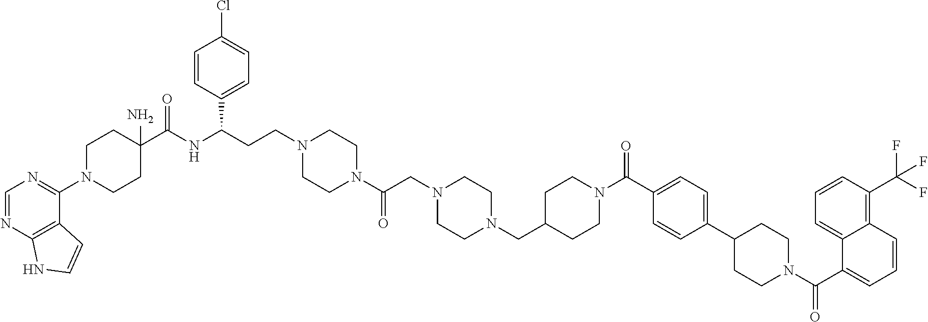 [SMILES: NC1(C(=O)N[C@@H](CCN2CCN(C(=O)CN3CCN(CC4CCN(C(=O)c5ccc(C6CCN(C(=O)c7cccc8c(C(F)(F)F)cccc78)CC6)cc5)CC4)CC3)CC2)c2ccc(Cl)cc2)CCN(c2ncnc3[nH]ccc23)CC1] |
| I-111 | 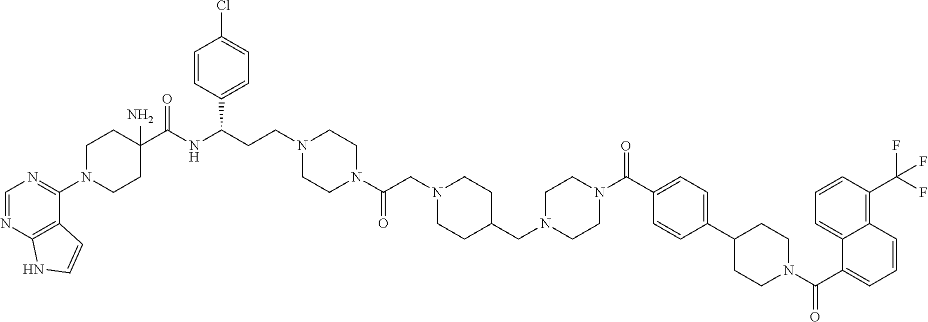 [SMILES: NC1(C(=O)N[C@@H](CCN2CCN(C(=O)CN3CCC(CN4CCN(C(=O)c5ccc(C6CCN(C(=O)c7cccc8c(C(F)(F)F)cccc78)CC6)cc5)CC4)CC3)CC2)c2ccc(Cl)cc2)CCN(c2ncnc3[nH]ccc23)CC1] |
| I-112 | 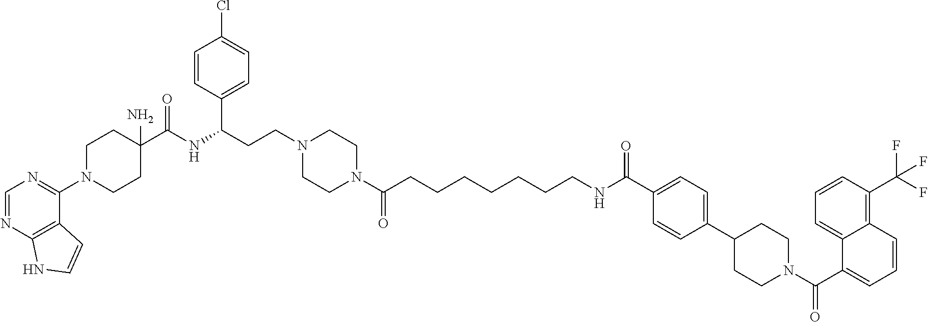 [SMILES: NC1(C(=O)N[C@@H](CCN2CCN(C(=O)CCCCCCCNC(=O)c3ccc(C4CCN(C(=O)c5cccc6c(C(F)(F)F)cccc56)CC4)cc3)CC2)c2ccc(Cl)cc2)CCN(c2ncnc3[nH]ccc23)CC1] |
| I-113 | 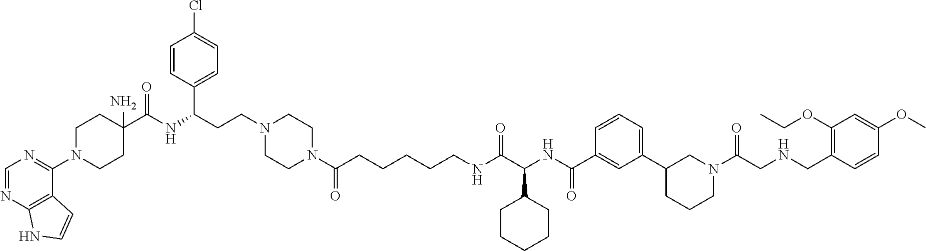 [SMILES: CCOc1cc(OC)ccc1CNCC(=O)N1CCCC(c2cccc(C(=O)N[C@H](C(=O)NCCCCCC(=O)N3CCN(CC[C@H](NC(=O)C4(N)CCN(c5ncnc6[nH]ccc56)CC4)c4ccc(Cl)cc4)CC3)C3CCCCC3)c2)C1] |
| I-114 | 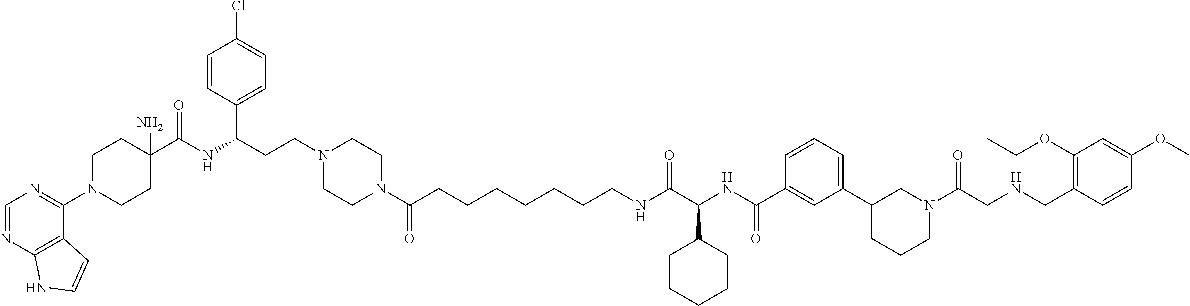 [SMILES: CCOc1cc(OC)ccc1CNCC(=O)N1CCCC(c2cccc(C(=O)N[C@H](C(=O)NCCCCCCCC(=O)N3CCN(CC[C@H](NC(=O)C4(N)CCN(c5ncnc6[nH]ccc56)CC4)c4ccc(Cl)cc4)CC3)C3CCCCC3)c2)C1] |
| I-115 | 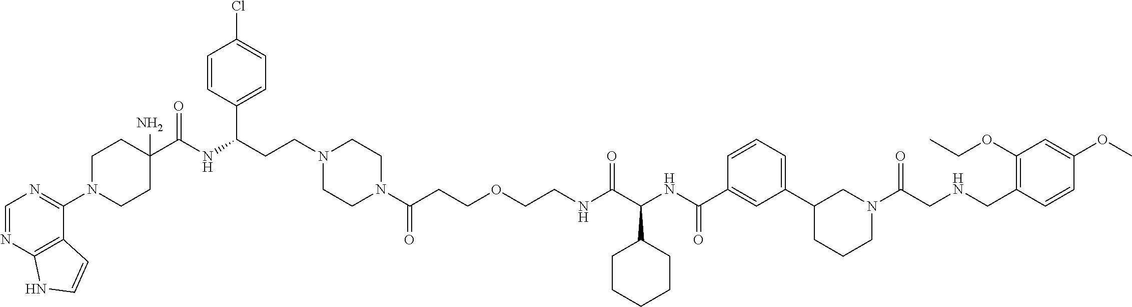 [SMILES: CCOc1cc(OC)ccc1CNCC(=O)N1CCCC(c2cccc(C(=O)N[C@H](C(=O)NCCOCCC(=O)N3CCN(CC[C@H](NC(=O)C4(N)CCN(c5ncnc6[nH]ccc56)CC4)c4ccc(Cl)cc4)CC3)C3CCCCC3)c2)C1] |
| I-116 | 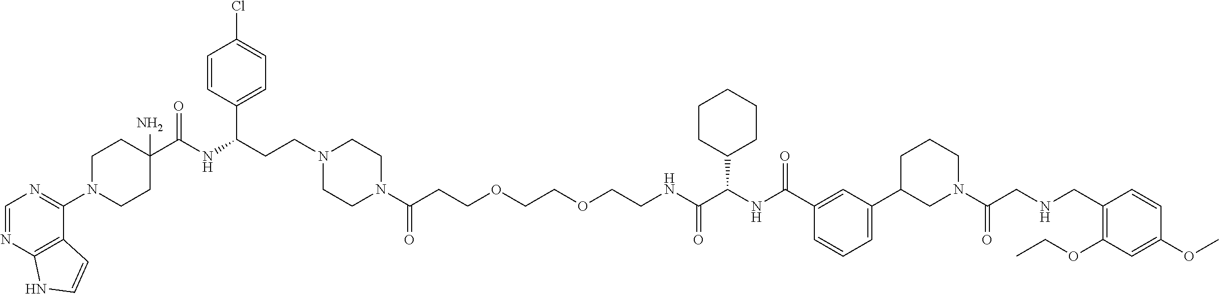 [SMILES: CCOc1cc(OC)ccc1CNCC(=O)N1CCCC(c2cccc(C(=O)N[C@H](C(=O)NCCOCCOCCC(=O)N3CCN(CC[C@H](NC(=O)C4(N)CCN(c5ncnc6[nH]ccc56)CC4)c4ccc(Cl)cc4)CC3)C3CCCCC3)c2)C1] |
| I-117 | 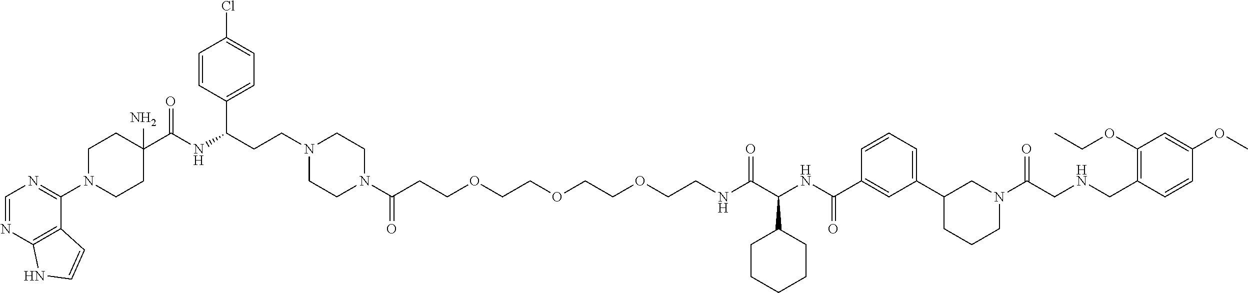 [SMILES: CCOc1cc(OC)ccc1CNCC(=O)N1CCCC(c2cccc(C(=O)N[C@H](C(=O)NCCOCCOCCOCCC(=O)N3CCN(CC[C@H](NC(=O)C4(N)CCN(c5ncnc6[nH]ccc56)CC4)c4ccc(Cl)cc4)CC3)C3CCCCC3)c2)C1] |
| I-118 | 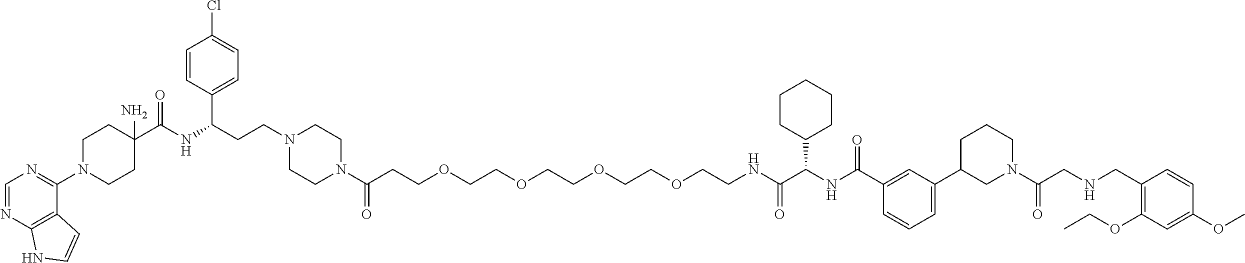 [SMILES: CCOc1cc(OC)ccc1CNCC(=O)N1CCCC(c2cccc(C(=O)N[C@H](C(=O)NCCOCCOCCOCCOCCC(=O)N3CCN(CC[C@H](NC(=O)C4(N)CCN(c5ncnc6[nH]ccc56)CC4)c4ccc(Cl)cc4)CC3)C3CCCCC3)c2)C1] |
| I-119 | 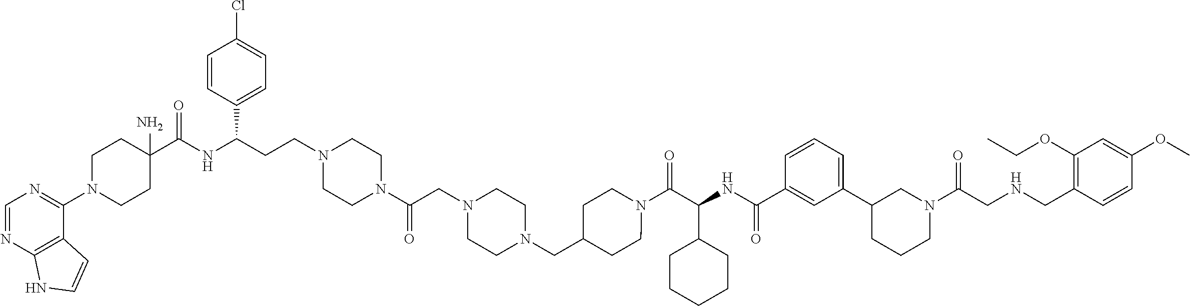 [SMILES: CCOc1cc(OC)ccc1CNCC(=O)N1CCCC(c2cccc(C(=O)N[C@H](C(=O)N3CCC(CN4CCN(CC(=O)N5CCN(CC[C@H](NC(=O)C6(N)CCN(c7ncnc8[nH]ccc78)CC6)c6ccc(Cl)cc6)CC5)CC4)CC3)C3CCCCC3)c2)C1] |
| I-120 | 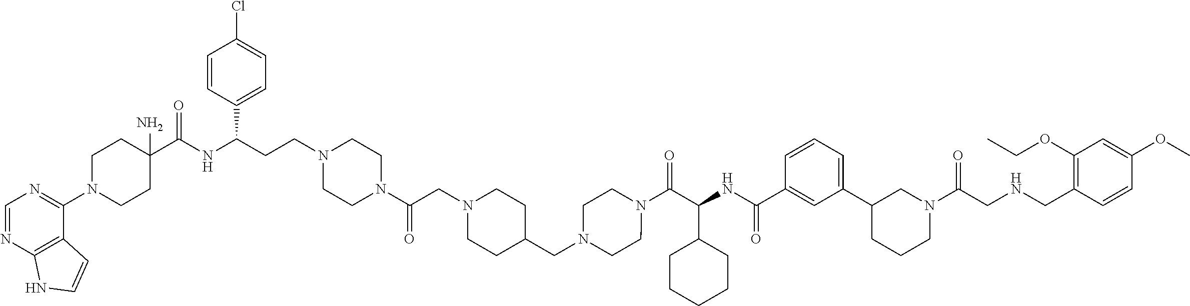 [SMILES: CCOc1cc(OC)ccc1CNCC(=O)N1CCCC(c2cccc(C(=O)N[C@H](C(=O)N3CCN(CC4CCN(CC(=O)N5CCN(CC[C@H](NC(=O)C6(N)CCN(c7ncnc8[nH]ccc78)CC6)c6ccc(Cl)cc6)CC5)CC4)CC3)C3CCCCC3)c2)C1] |
| I-121 | 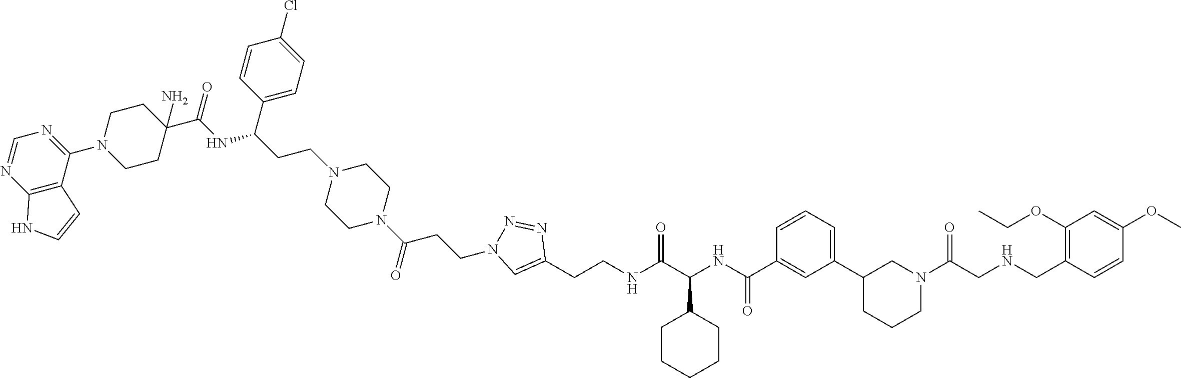 [SMILES: CCOc1cc(OC)ccc1CNCC(=O)N1CCCC(c2cccc(C(=O)N[C@H](C(=O)NCCc3cn(CCC(=O)N4CCN(CC[C@H](NC(=O)C5(N)CCN(c6ncnc7[nH]ccc67)CC5)c5ccc(Cl)cc5)CC4)nn3)C3CCCCC3)c2)C1] |
| I-122 | 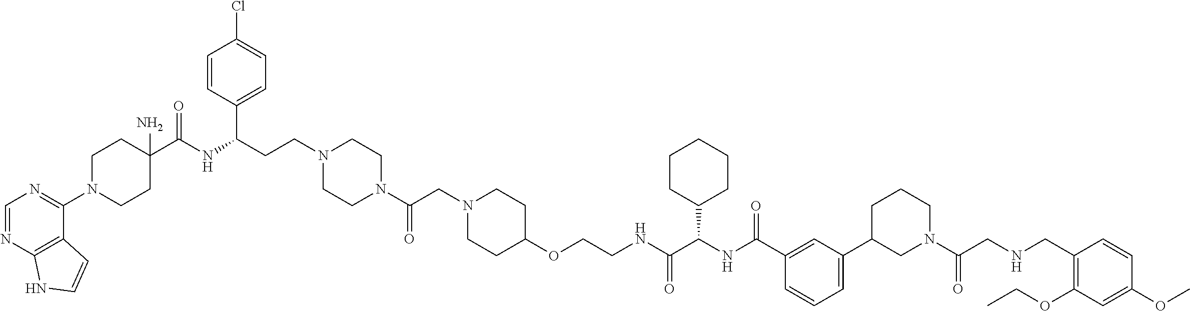 [SMILES: CCOc1cc(OC)ccc1CNCC(=O)N1CCCC(c2cccc(C(=O)N[C@H](C(=O)NCCOC3CCN(CC(=O)N4CCN(CC[C@H](NC(=O)C5(N)CCN(c6ncnc7[nH]ccc67)CC5)c5ccc(Cl)cc5)CC4)CC3)C3CCCCC3)c2)C1] |
| I-123 | 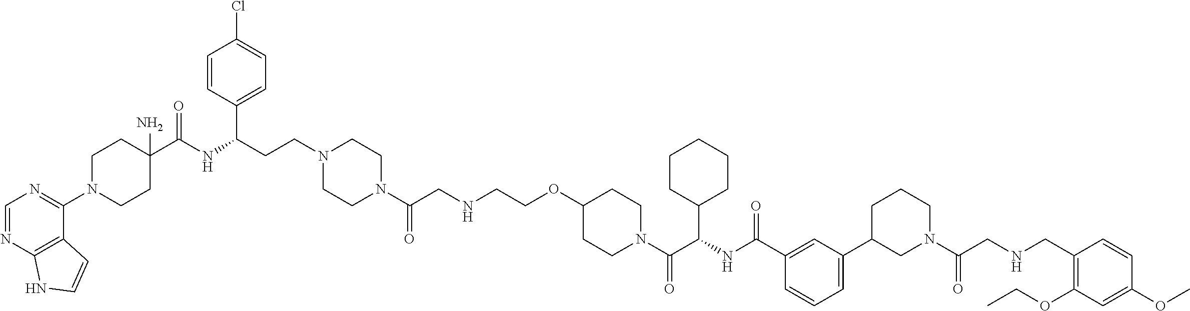 [SMILES: CCOc1cc(OC)ccc1CNCC(=O)N1CCCC(c2cccc(C(=O)N[C@H](C(=O)N3CCC(OCCNCC(=O)N4CCN(CC[C@H](NC(=O)C5(N)CCN(c6ncnc7[nH]ccc67)CC5)c5ccc(Cl)cc5)CC4)CC3)C3CCCCC3)c2)C1] |
| I-124 | 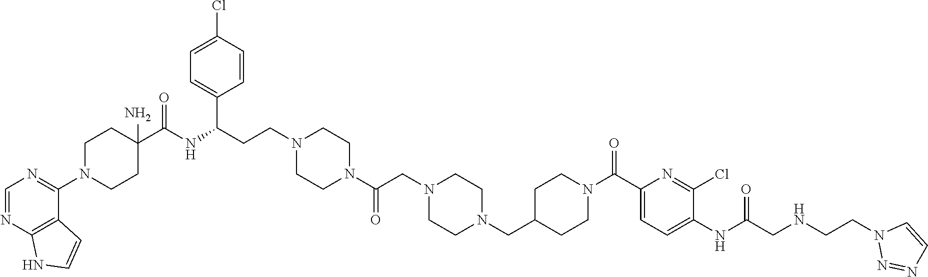 [SMILES: NC1(C(=O)N[C@@H](CCN2CCN(C(=O)CN3CCN(CC4CCN(C(=O)c5ccc(NC(=O)CNCCn6ccnn6)c(Cl)n5)CC4)CC3)CC2)c2ccc(Cl)cc2)CCN(c2ncnc3[nH]ccc23)CC1] |
| I-125 | 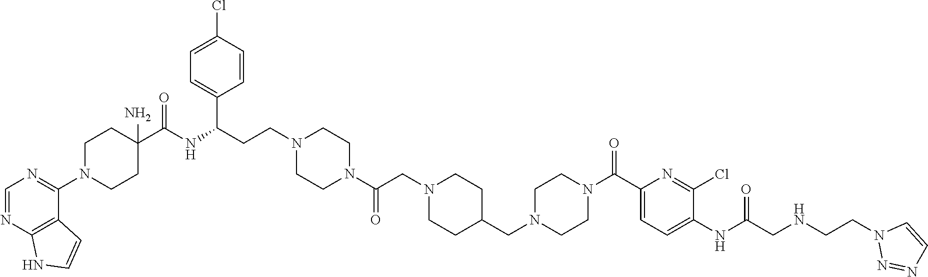 [SMILES: NC1(C(=O)N[C@@H](CCN2CCN(C(=O)CN3CCC(CN4CCN(C(=O)c5ccc(NC(=O)CNCCn6ccnn6)c(Cl)n5)CC4)CC3)CC2)c2ccc(Cl)cc2)CCN(c2ncnc3[nH]ccc23)CC1] |
| I-126 | 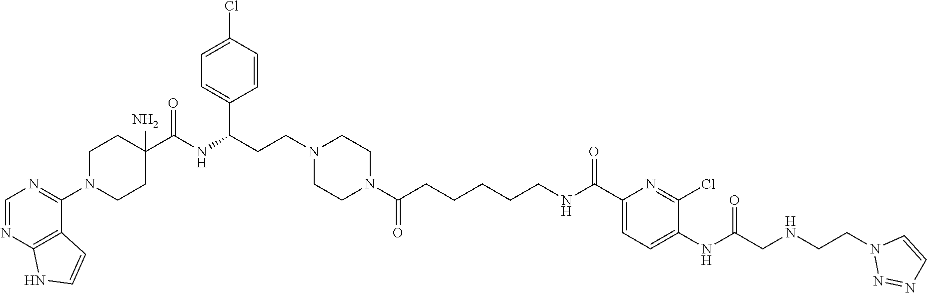 [SMILES: NC1(C(=O)N[C@@H](CCN2CCN(C(=O)CCCCCNC(=O)c3ccc(NC(=O)CNCCn4ccnn4)c(Cl)n3)CC2)c2ccc(Cl)cc2)CCN(c2ncnc3[nH]ccc23)CC1] |
| I-127 | 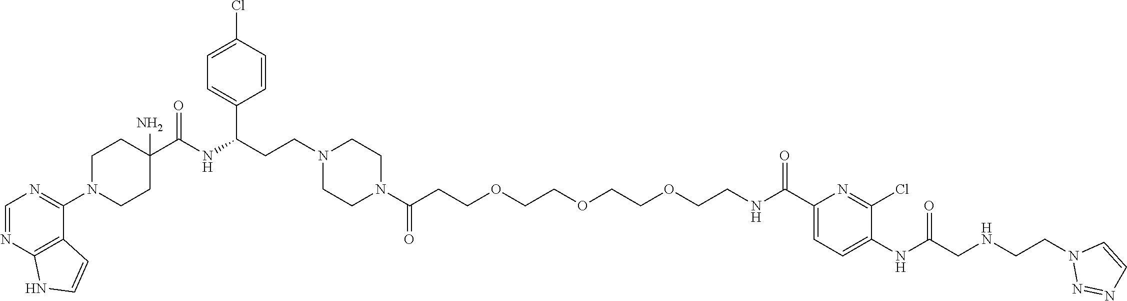 [SMILES: NC1(C(=O)N[C@@H](CCN2CCN(C(=O)CCOCCOCCOCCNC(=O)c3ccc(NC(=O)CNCCn4ccnn4)c(Cl)n3)CC2)c2ccc(Cl)cc2)CCN(c2ncnc3[nH]ccc23)CC1] |
| I-128 | 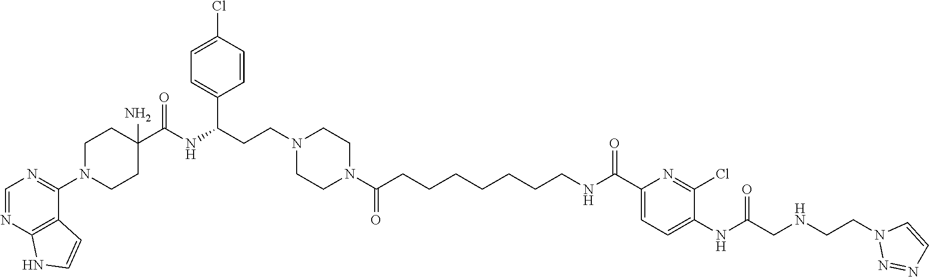 [SMILES: NC1(C(=O)N[C@@H](CCN2CCN(C(=O)CCCCCCCNC(=O)c3ccc(NC(=O)CNCCn4ccnn4)c(Cl)n3)CC2)c2ccc(Cl)cc2)CCN(c2ncnc3[nH]ccc23)CC1] |
| I-129 | 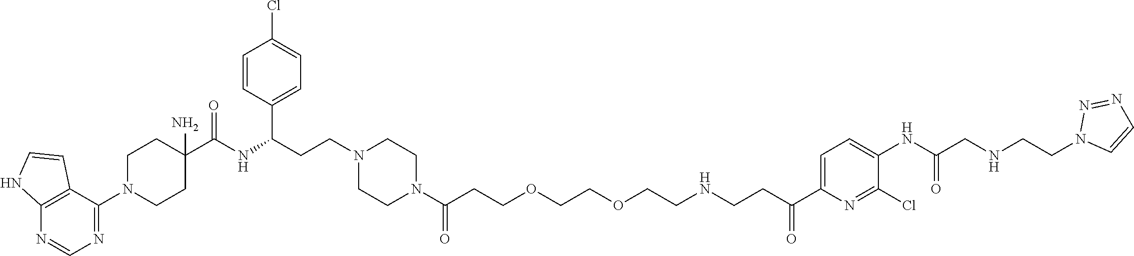 [SMILES: NC1(C(=O)N[C@@H](CCN2CCN(C(=O)CCOCCOCCNCCC(=O)c3ccc(NC(=O)CNCCn4ccnn4)c(Cl)n3)CC2)c2ccc(Cl)cc2)CCN(c2ncnc3[nH]ccc23)CC1] |
| I-130 | 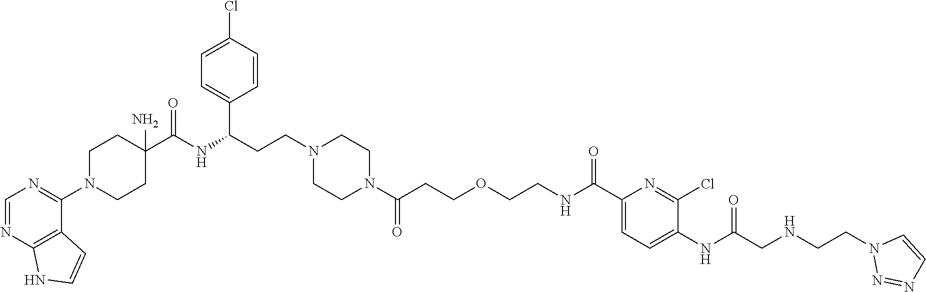 [SMILES: NC1(C(=O)N[C@@H](CCN2CCN(C(=O)CCOCCNC(=O)c3ccc(NC(=O)CNCCn4ccnn4)c(Cl)n3)CC2)c2ccc(Cl)cc2)CCN(c2ncnc3[nH]ccc23)CC1] |
| I-131 | 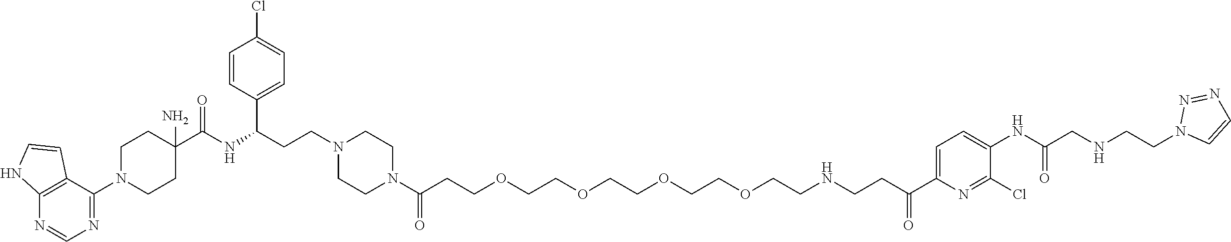 [SMILES: NC1(C(=O)N[C@@H](CCN2CCN(C(=O)CCOCCOCCOCCOCCNCCC(=O)c3ccc(NC(=O)CNCCn4ccnn4)c(Cl)n3)CC2)c2ccc(Cl)cc2)CCN(c2ncnc3[nH]ccc23)CC1] |
| I-132 | 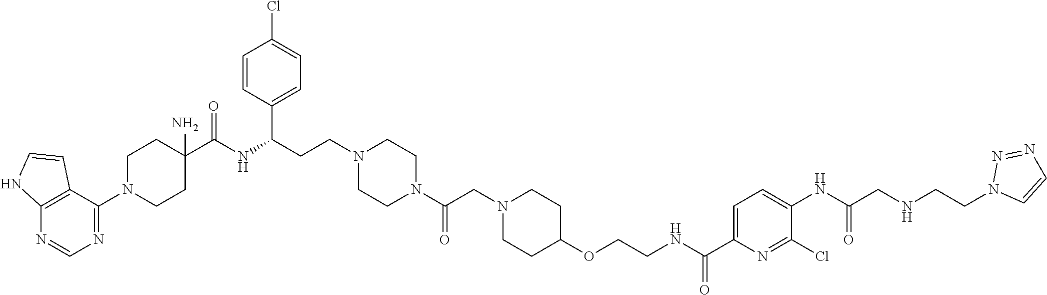 [SMILES: NC1(C(=O)N[C@@H](CCN2CCN(C(=O)CN3CCC(OCCNC(=O)c4ccc(NC(=O)CNCCn5ccnn5)c(Cl)n4)CC3)CC2)c2ccc(Cl)cc2)CCN(c2ncnc3[nH]ccc23)CC1] |
| I-133 | 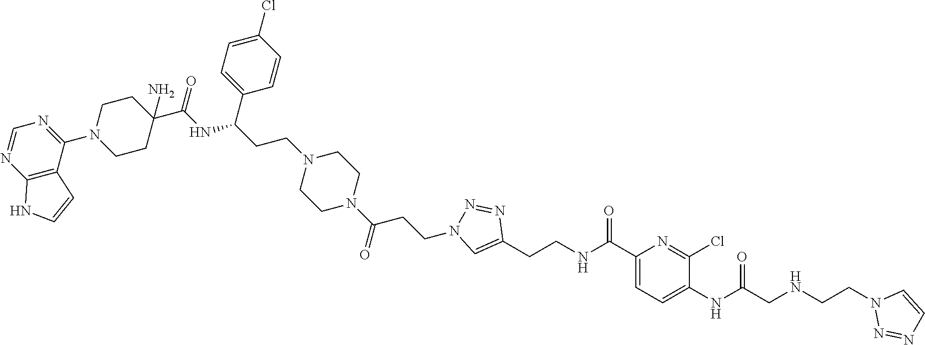 [SMILES: NC1(C(=O)N[C@@H](CCN2CCN(C(=O)CCn3cc(CCNC(=O)c4ccc(NC(=O)CNCCn5ccnn5)c(Cl)n4)nn3)CC2)c2ccc(Cl)cc2)CCN(c2ncnc3[nH]ccc23)CC1] |
| I-134 | 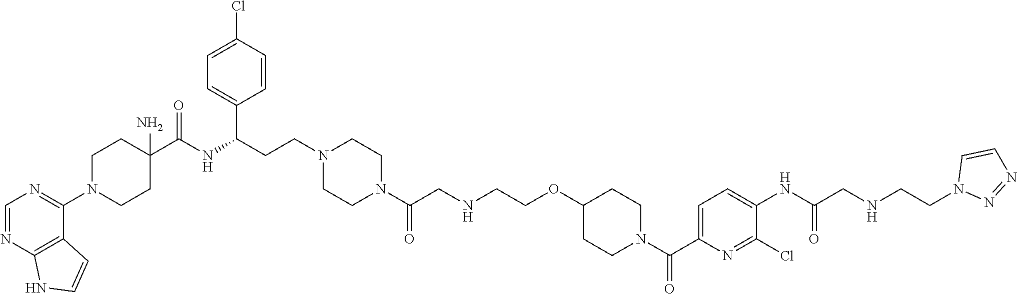 [SMILES: NC1(C(=O)N[C@@H](CCN2CCN(C(=O)CNCCOC3CCN(C(=O)c4ccc(NC(=O)CNCCn5ccnn5)c(Cl)n4)CC3)CC2)c2ccc(Cl)cc2)CCN(c2ncnc3[nH]ccc23)CC1] |
| I-135 | 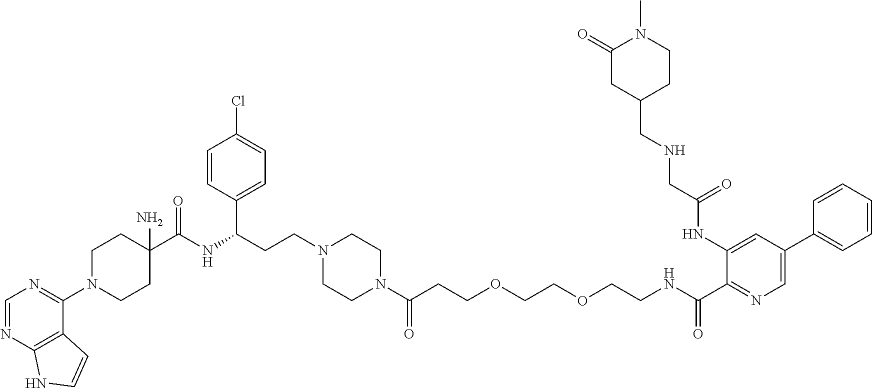 [SMILES: CN1CCC(CNCC(=O)Nc2cc(-c3ccccc3)cnc2C(=O)NCCOCCOCCC(=O)N2CCN(CC[C@H](NC(=O)C3(N)CCN(c4ncnc5[nH]ccc45)CC3)c3ccc(Cl)cc3)CC2)CC1=O] |
| I-136 | 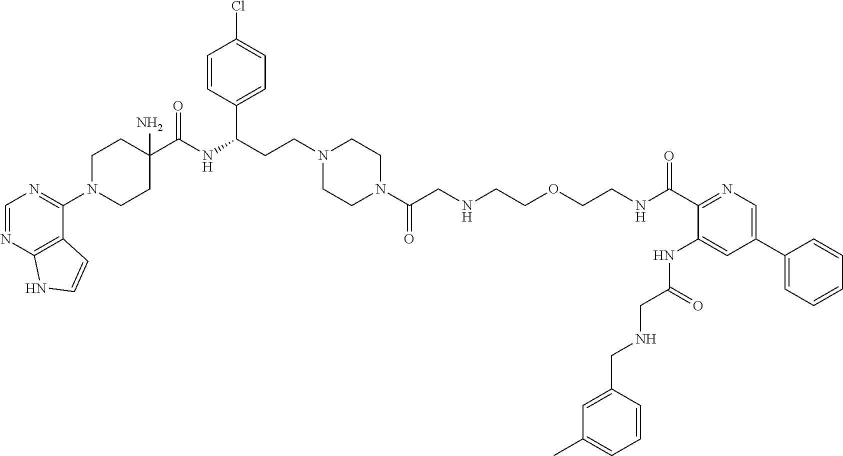 [SMILES: Cc1cccc(CNCC(=O)Nc2cc(-c3ccccc3)cnc2C(=O)NCCOCCNCC(=O)N2CCN(CC[C@H](NC(=O)C3(N)CCN(c4ncnc5[nH]ccc45)CC3)c3ccc(Cl)cc3)CC2)c1] |
| I-137 | 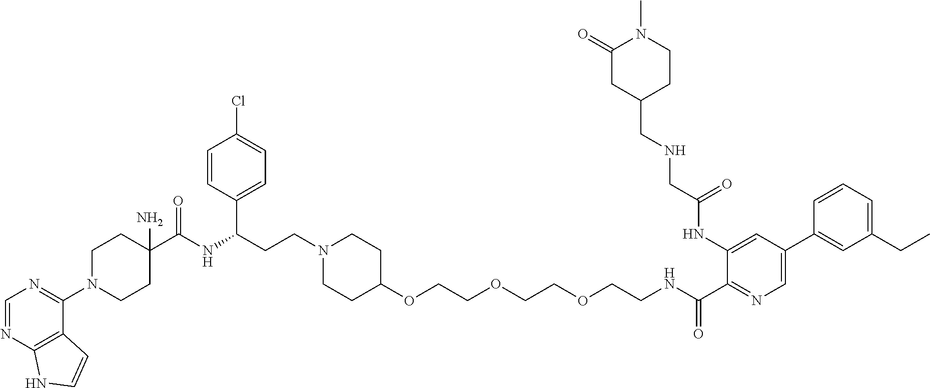 [SMILES: CCc1cccc(-c2cnc(C(=O)NCCOCCOCCOC3CCN(CC[C@H](NC(=O)C4(N)CCN(c5ncnc6[nH]ccc56)CC4)c4ccc(Cl)cc4)CC3)c(NC(=O)CNCC3CCN(C)C(=O)C3)c2)c1] |
| I-138 | 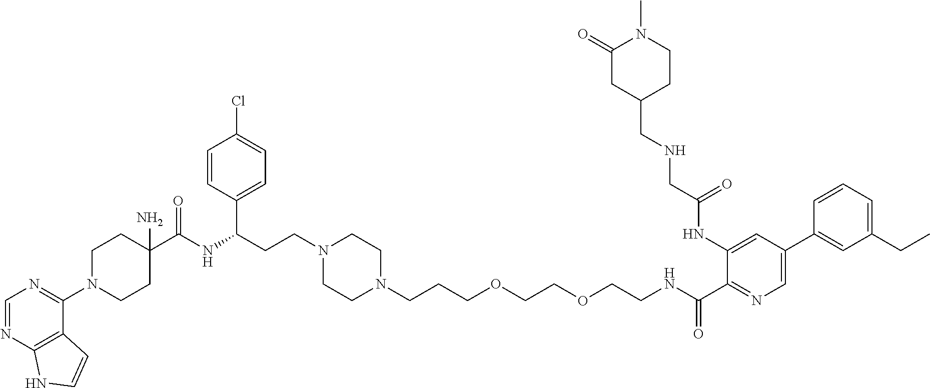 [SMILES: CCc1cccc(-c2cnc(C(=O)NCCOCCOCCCN3CCN(CC[C@H](NC(=O)C4(N)CCN(c5ncnc6[nH]ccc56)CC4)c4ccc(Cl)cc4)CC3)c(NC(=O)CNCC3CCN(C)C(=O)C3)c2)c1] |
| I-139 | 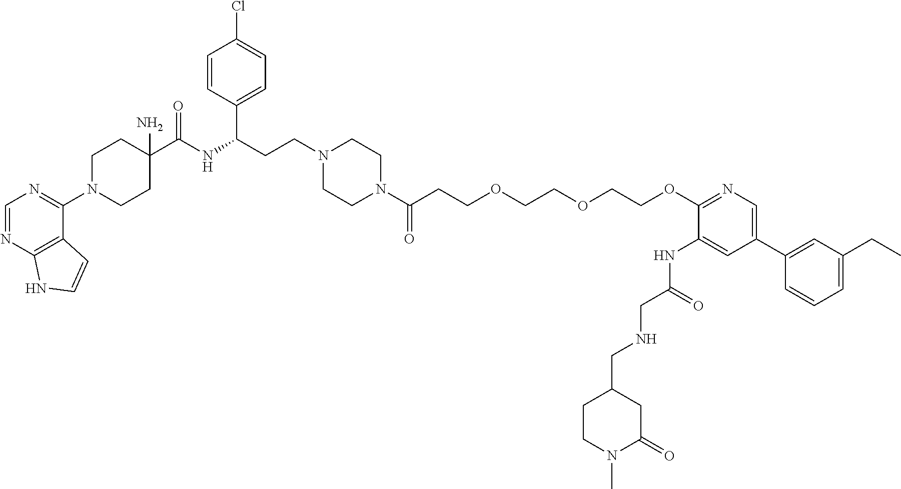 [SMILES: CCc1cccc(-c2cnc(OCCOCCOCCC(=O)N3CCN(CC[C@H](NC(=O)C4(N)CCN(c5ncnc6[nH]ccc56)CC4)c4ccc(Cl)cc4)CC3)c(NC(=O)CNCC3CCN(C)C(=O)C3)c2)c1] |
| I-140 | 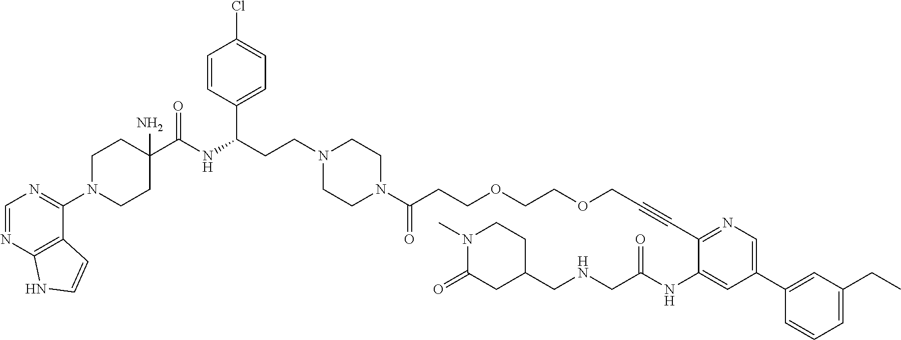 [SMILES: CCc1cccc(-c2cnc(C#CCOCCOCCC(=O)N3CCN(CC[C@H](NC(=O)C4(N)CCN(c5ncnc6[nH]ccc56)CC4)c4ccc(Cl)cc4)CC3)c(NC(=O)CNCC3CCN(C)C(=O)C3)c2)c1] |
| I-141 | 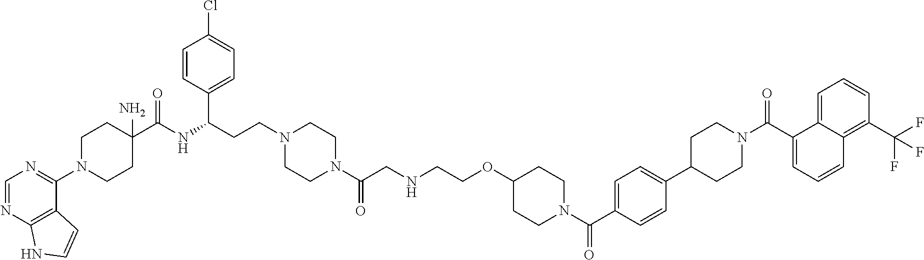 [SMILES: NC1(C(=O)N[C@@H](CCN2CCN(C(=O)CNCCOC3CCN(C(=O)c4ccc(C5CCN(C(=O)c6cccc7c(C(F)(F)F)cccc67)CC5)cc4)CC3)CC2)c2ccc(Cl)cc2)CCN(c2ncnc3[nH]ccc23)CC1] |
| I-142 | 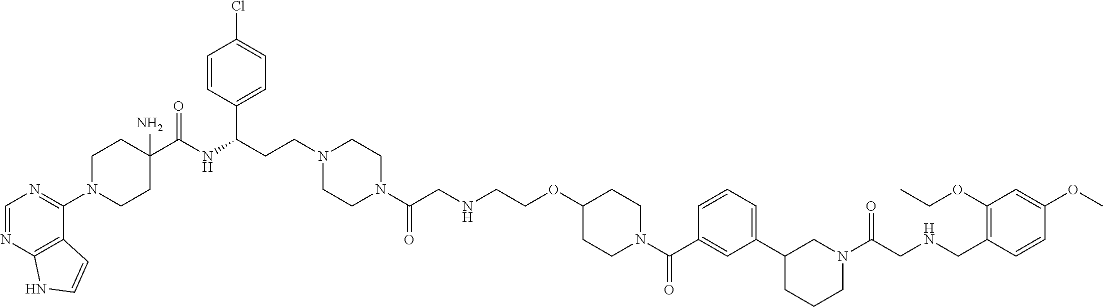 [SMILES: CCOc1cc(OC)ccc1CNCC(=O)N1CCCC(c2cccc(C(=O)N3CCC(OCCNCC(=O)N4CCN(CC[C@H](NC(=O)C5(N)CCN(c6ncnc7[nH]ccc67)CC5)c5ccc(Cl)cc5)CC4)CC3)c2)C1] |
| I-144 | 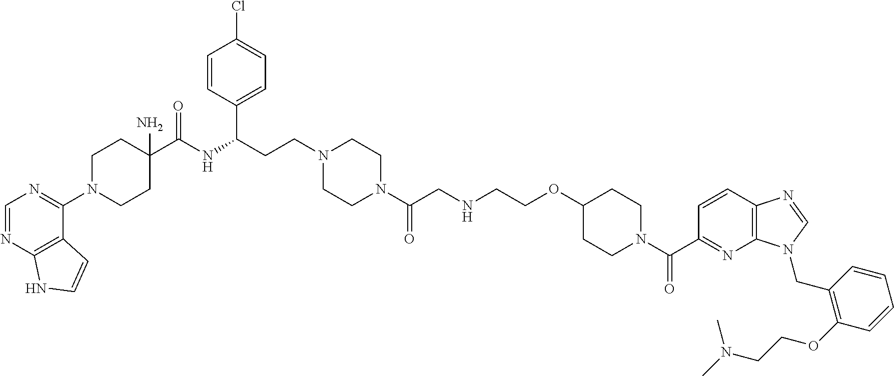 [SMILES: CN(C)CCOc1ccccc1Cn1cnc2ccc(C(=O)N3CCC(OCCNCC(=O)N4CCN(CC[C@H](NC(=O)C5(N)CCN(c6ncnc7[nH]ccc67)CC5)c5ccc(Cl)cc5)CC4)CC3)nc21] |
| I-145 | 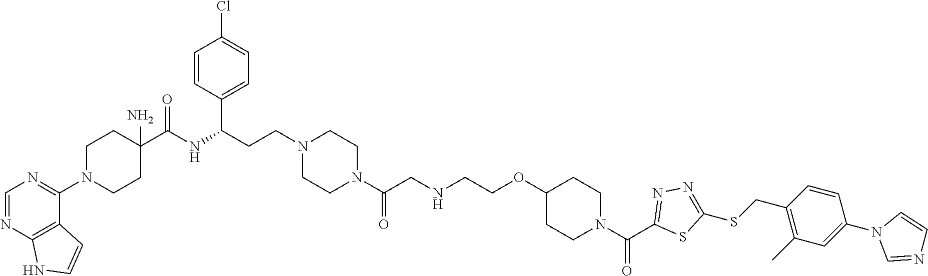 [SMILES: Cc1cc(-n2ccnc2)ccc1CSc1nnc(C(=O)N2CCC(OCCNCC(=O)N3CCN(CC[C@H](NC(=O)C4(N)CCN(c5ncnc6[nH]ccc56)CC4)c4ccc(Cl)cc4)CC3)CC2)s1] |
| I-146 | 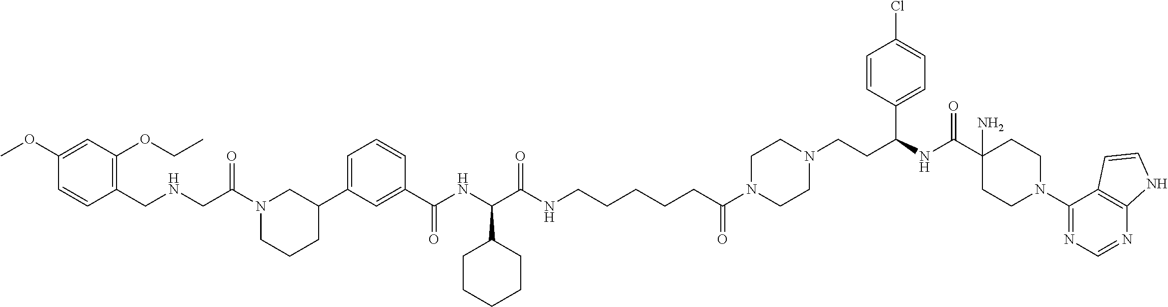 [SMILES: CCOc1cc(OC)ccc1CNCC(=O)N1CCCC(c2cccc(C(=O)N[C@@H](C(=O)NCCCCCC(=O)N3CCN(CC[C@H](NC(=O)C4(N)CCN(c5ncnc6[nH]ccc56)CC4)c4ccc(Cl)cc4)CC3)C3CCCCC3)c2)C1] |
| I-147 | 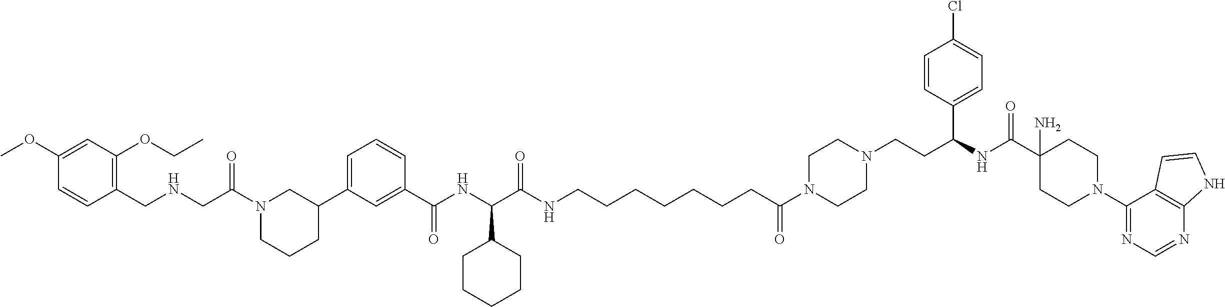 [SMILES: CCOc1cc(OC)ccc1CNCC(=O)N1CCCC(c2cccc(C(=O)N[C@@H](C(=O)NCCCCCCCC(=O)N3CCN(CC[C@H](NC(=O)C4(N)CCN(c5ncnc6[nH]ccc56)CC4)c4ccc(Cl)cc4)CC3)C3CCCCC3)c2)C1] |
| I-148 | 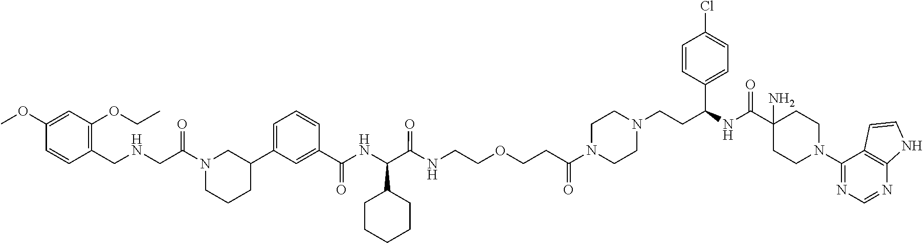 [SMILES: CCOc1cc(OC)ccc1CNCC(=O)N1CCCC(c2cccc(C(=O)N[C@@H](C(=O)NCCOCCC(=O)N3CCN(CC[C@H](NC(=O)C4(N)CCN(c5ncnc6[nH]ccc56)CC4)c4ccc(Cl)cc4)CC3)C3CCCCC3)c2)C1] |
| I-149 | 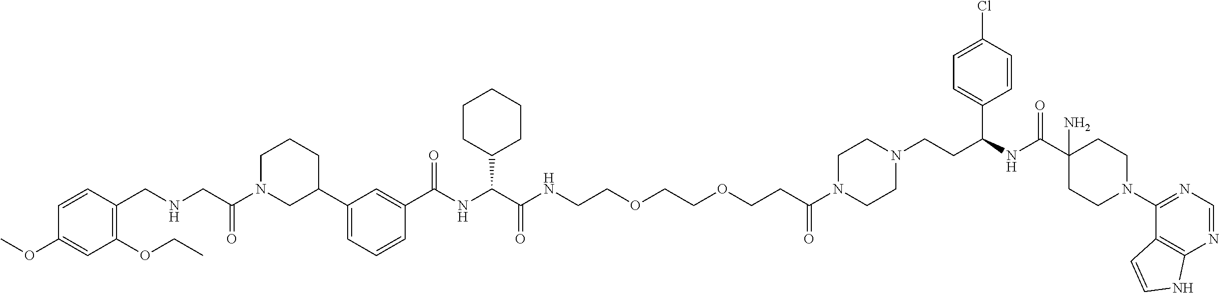 [SMILES: CCOc1cc(OC)ccc1CNCC(=O)N1CCCC(c2cccc(C(=O)N[C@@H](C(=O)NCCOCCOCCC(=O)N3CCN(CC[C@H](NC(=O)C4(N)CCN(c5ncnc6[nH]ccc56)CC4)c4ccc(Cl)cc4)CC3)C3CCCCC3)c2)C1] |
| I-150 | 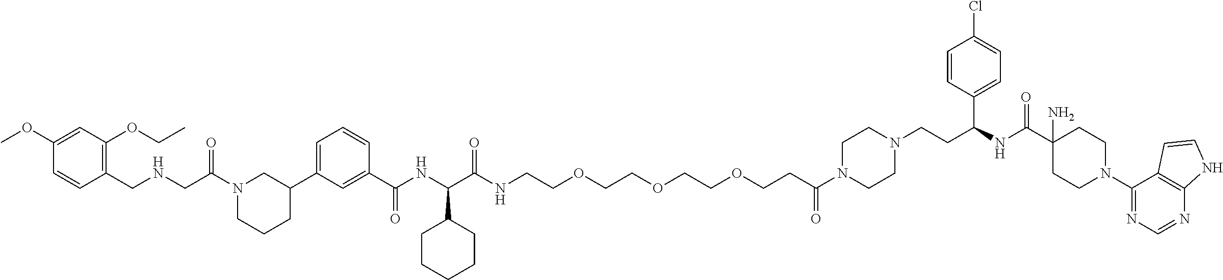 [SMILES: CCOc1cc(OC)ccc1CNCC(=O)N1CCCC(c2cccc(C(=O)N[C@@H](C(=O)NCCOCCOCCOCCC(=O)N3CCN(CC[C@H](NC(=O)C4(N)CCN(c5ncnc6[nH]ccc56)CC4)c4ccc(Cl)cc4)CC3)C3CCCCC3)c2)C1] |
| I-151 | 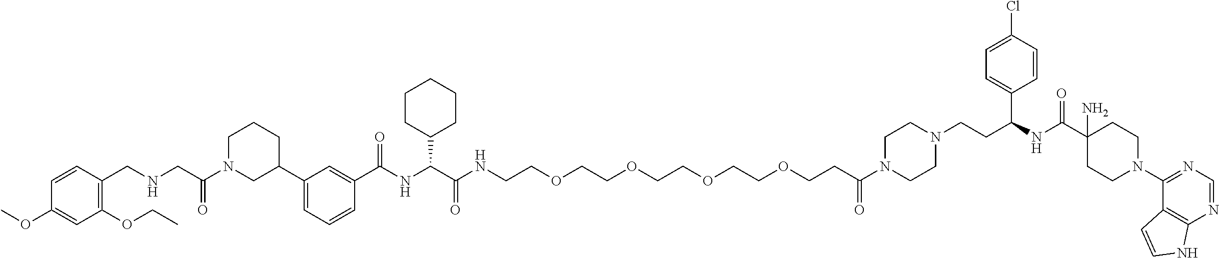 [SMILES: CCOc1cc(OC)ccc1CNCC(=O)N1CCCC(c2cccc(C(=O)N[C@@H](C(=O)NCCOCCOCCOCCOCCC(=O)N3CCN(CC[C@H](NC(=O)C4(N)CCN(c5ncnc6[nH]ccc56)CC4)c4ccc(Cl)cc4)CC3)C3CCCCC3)c2)C1] |
| I-152 | 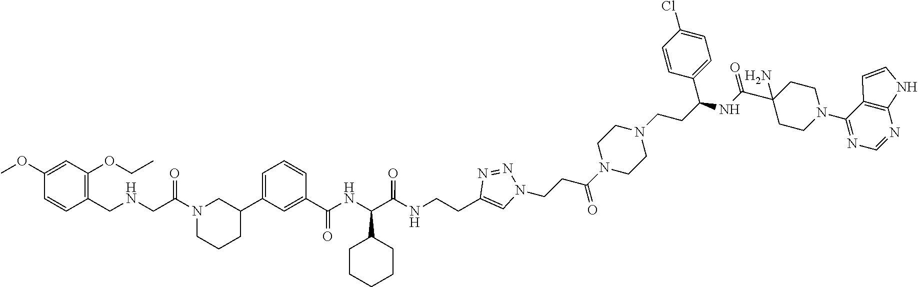 [SMILES: CCOc1cc(OC)ccc1CNCC(=O)N1CCCC(c2cccc(C(=O)N[C@@H](C(=O)NCCc3cn(CCC(=O)N4CCN(CC[C@H](NC(=O)C5(N)CCN(c6ncnc7[nH]ccc67)CC5)c5ccc(Cl)cc5)CC4)nn3)C3CCCCC3)c2)C1] |
| I-153 | 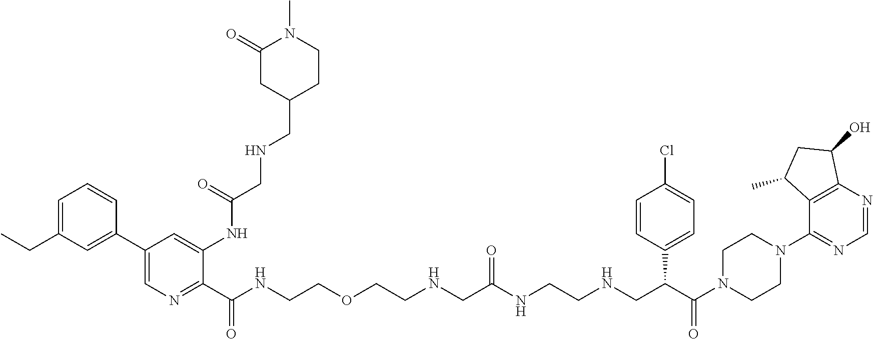 [SMILES: CCc1cccc(-c2cnc(C(=O)NCCOCCNCC(=O)NCCNC[C@@H](C(=O)N3CCN(c4ncnc5c4[C@H](C)C[C@H]5O)CC3)c3ccc(Cl)cc3)c(NC(=O)CNCC3CCN(C)C(=O)C3)c2)c1] |
| I-154 | 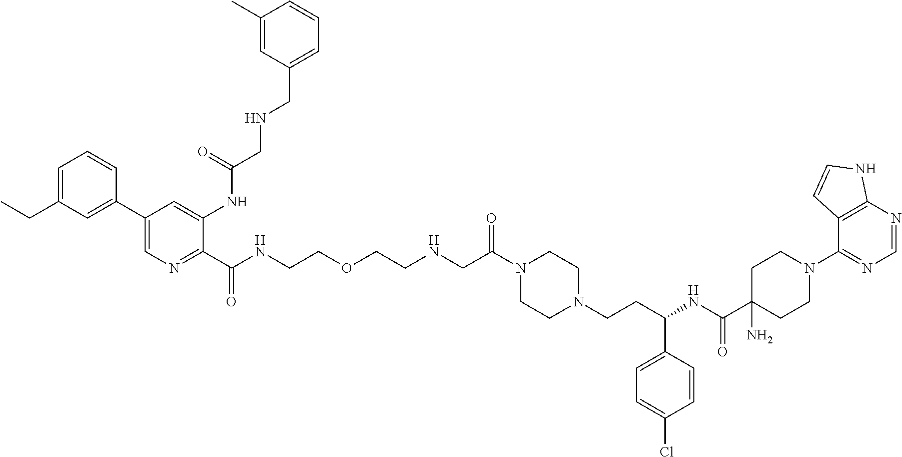 [SMILES: CCc1cccc(-c2cnc(C(=O)NCCOCCNCC(=O)N3CCN(CC[C@H](NC(=O)C4(N)CCN(c5ncnc6[nH]ccc56)CC4)c4ccc(Cl)cc4)CC3)c(NC(=O)CNCc3cccc(C)c3)c2)c1] |
| I-155 | 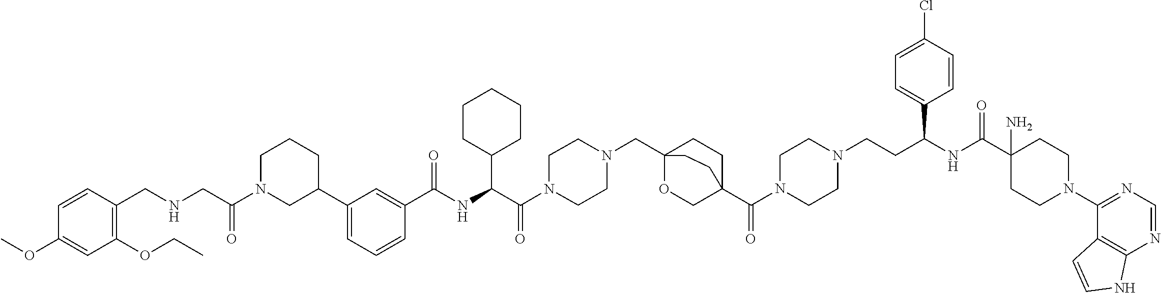 [SMILES: CCOc1cc(OC)ccc1CNCC(=O)N1CCCC(c2cccc(C(=O)N[C@H](C(=O)N3CCN(CC45CCC(C(=O)N6CCN(CC[C@H](NC(=O)C7(N)CCN(c8ncnc9[nH]ccc89)CC7)c7ccc(Cl)cc7)CC6)(CC4)CO5)CC3)C3CCCCC3)c2)C1] |
| I-156 | 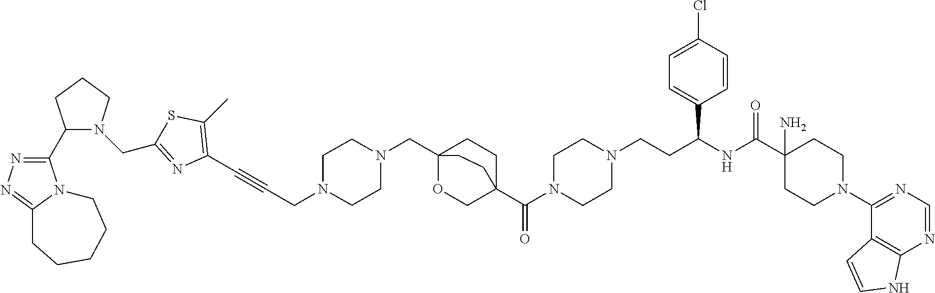 [SMILES: Cc1sc(CN2CCCC2c2nnc3n2CCCCC3)nc1C#CCN1CCN(CC23CCC(C(=O)N4CCN(CC[C@H](NC(=O)C5(N)CCN(c6ncnc7[nH]ccc67)CC5)c5ccc(Cl)cc5)CC4)(CC2)CO3)CC1] |
| I-157 | 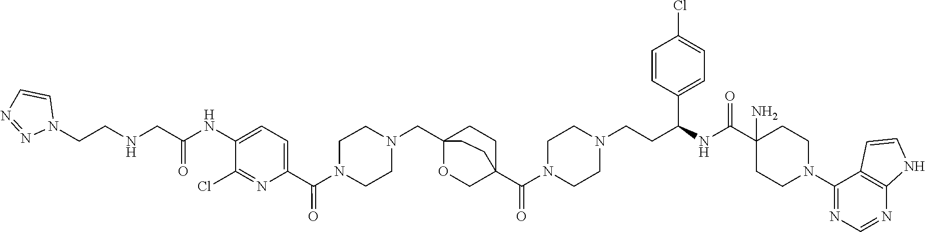 [SMILES: NC1(C(=O)N[C@@H](CCN2CCN(C(=O)C34CCC(CN5CCN(C(=O)c6ccc(NC(=O)CNCCn7ccnn7)c(Cl)n6)CC5)(CC3)OC4)CC2)c2ccc(Cl)cc2)CCN(c2ncnc3[nH]ccc23)CC1] |
| I-158 | 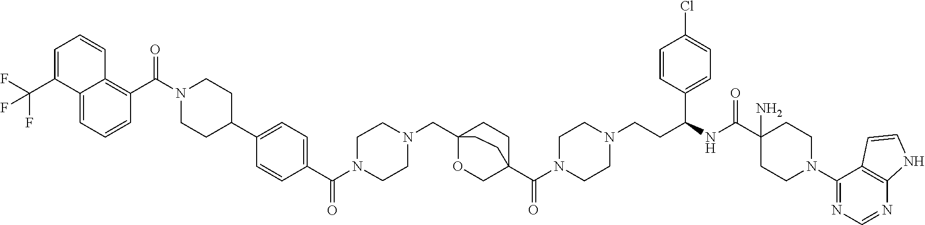 [SMILES: NC1(C(=O)N[C@@H](CCN2CCN(C(=O)C34CCC(CN5CCN(C(=O)c6ccc(C7CCN(C(=O)c8cccc9c(C(F)(F)F)cccc89)CC7)cc6)CC5)(CC3)OC4)CC2)c2ccc(Cl)cc2)CCN(c2ncnc3[nH]ccc23)CC1] |
| I-159 | 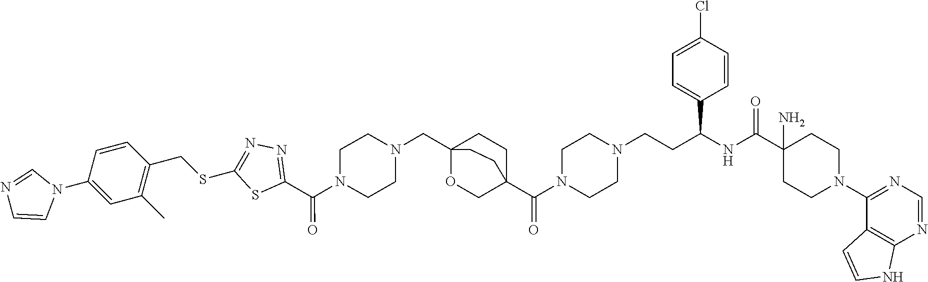 [SMILES: Cc1cc(-n2ccnc2)ccc1CSc1nnc(C(=O)N2CCN(CC34CCC(C(=O)N5CCN(CC[C@H](NC(=O)C6(N)CCN(c7ncnc8[nH]ccc78)CC6)c6ccc(Cl)cc6)CC5)(CC3)CO4)CC2)s1] |
| I-160 | 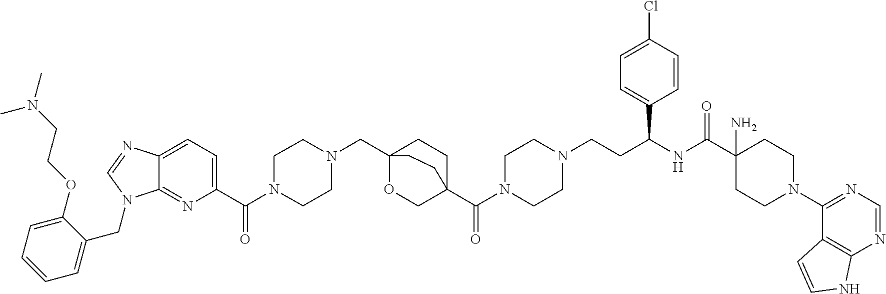 [SMILES: CN(C)CCOc1ccccc1Cn1cnc2ccc(C(=O)N3CCN(CC45CCC(C(=O)N6CCN(CC[C@H](NC(=O)C7(N)CCN(c8ncnc9[nH]ccc89)CC7)c7ccc(Cl)cc7)CC6)(CC4)CO5)CC3)nc21] |
| I-161 | 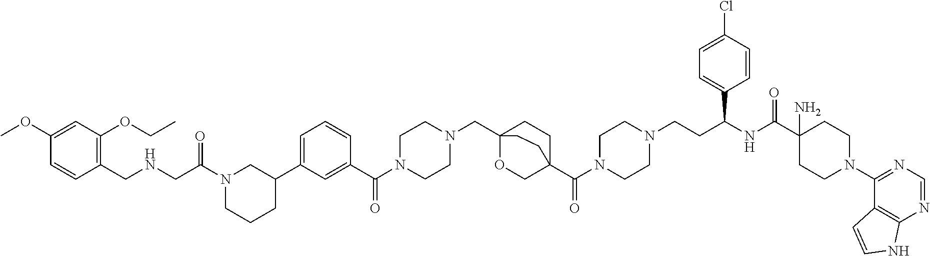 [SMILES: CCOc1cc(OC)ccc1CNCC(=O)N1CCCC(c2cccc(C(=O)N3CCN(CC45CCC(C(=O)N6CCN(CC[C@H](NC(=O)C7(N)CCN(c8ncnc9[nH]ccc89)CC7)c7ccc(Cl)cc7)CC6)(CC4)CO5)CC3)c2)C1] |
| I-162 | 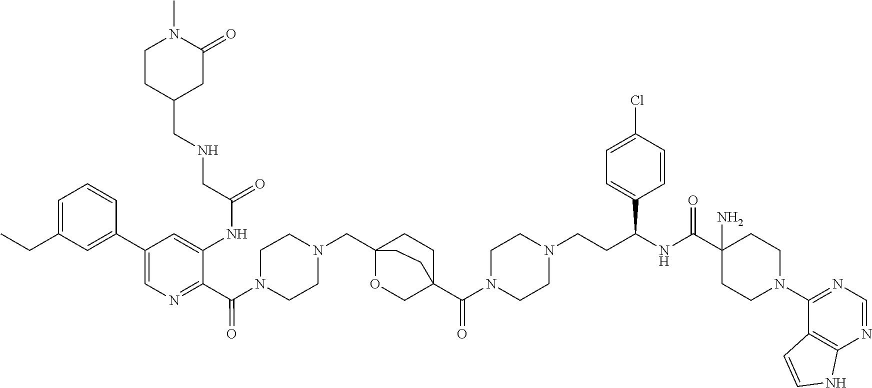 [SMILES: CCc1cccc(-c2cnc(C(=O)N3CCN(CC45CCC(C(=O)N6CCN(CC[C@H](NC(=O)C7(N)CCN(c8ncnc9[nH]ccc89)CC7)c7ccc(Cl)cc7)CC6)(CC4)CO5)CC3)c(NC(=O)CNCC3CCN(C)C(=O)C3)c2)c1] |
| I-163 | 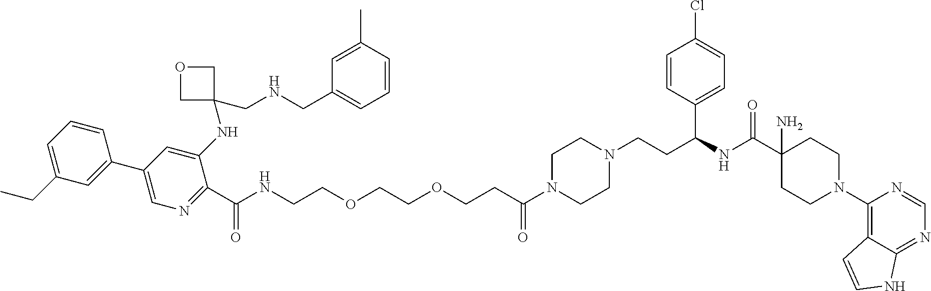 [SMILES: CCc1cccc(-c2cnc(C(=O)NCCOCCOCCC(=O)N3CCN(CC[C@H](NC(=O)C4(N)CCN(c5ncnc6[nH]ccc56)CC4)c4ccc(Cl)cc4)CC3)c(NC3(CNCc4cccc(C)c4)COC3)c2)c1] |
| I-164 | 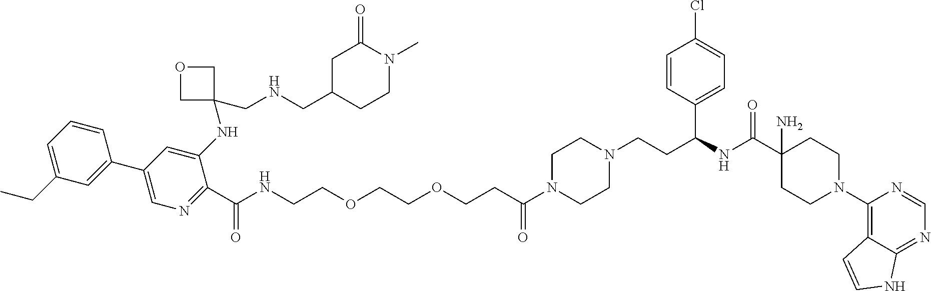 [SMILES: CCc1cccc(-c2cnc(C(=O)NCCOCCOCCC(=O)N3CCN(CC[C@H](NC(=O)C4(N)CCN(c5ncnc6[nH]ccc56)CC4)c4ccc(Cl)cc4)CC3)c(NC3(CNCC4CCN(C)C(=O)C4)COC3)c2)c1] |
| I-165 | 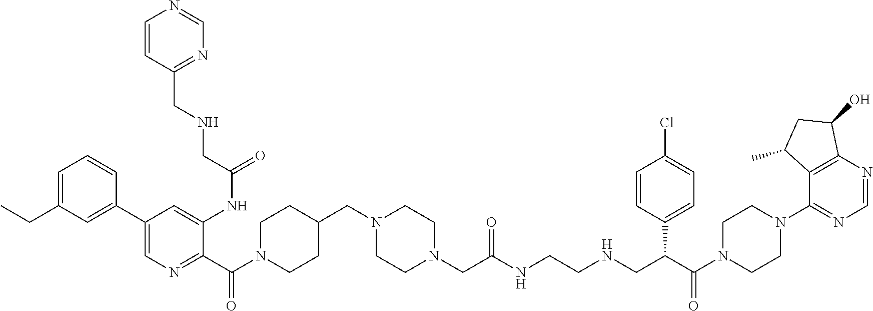 [SMILES: CCc1cccc(-c2cnc(C(=O)N3CCC(CN4CCN(CC(=O)NCCNC[C@@H](C(=O)N5CCN(c6ncnc7c6[C@H](C)C[C@H]7O)CC5)c5ccc(Cl)cc5)CC4)CC3)c(NC(=O)CNCc3ccncn3)c2)c1] |
| I-166 | 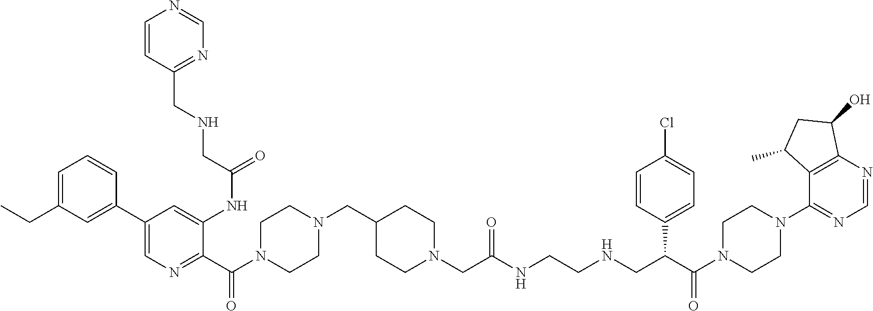 [SMILES: CCc1cccc(-c2cnc(C(=O)N3CCN(CC4CCN(CC(=O)NCCNC[C@@H](C(=O)N5CCN(c6ncnc7c6[C@H](C)C[C@H]7O)CC5)c5ccc(Cl)cc5)CC4)CC3)c(NC(=O)CNCc3ccncn3)c2)c1] |
| I-167 | 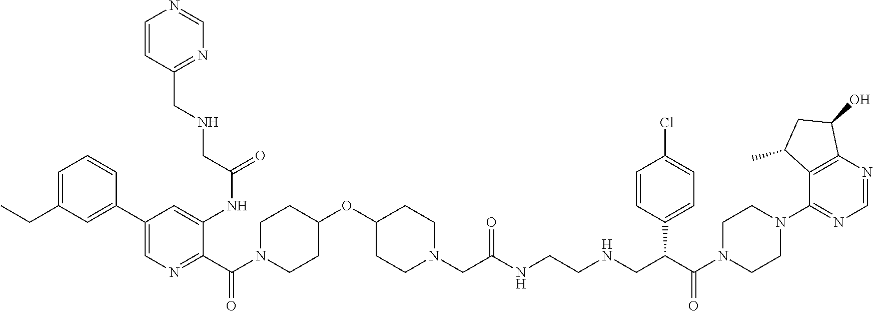 [SMILES: CCc1cccc(-c2cnc(C(=O)N3CCC(OC4CCN(CC(=O)NCCNC[C@@H](C(=O)N5CCN(c6ncnc7c6[C@H](C)C[C@H]7O)CC5)c5ccc(Cl)cc5)CC4)CC3)c(NC(=O)CNCc3ccncn3)c2)c1] |
| I-168 | 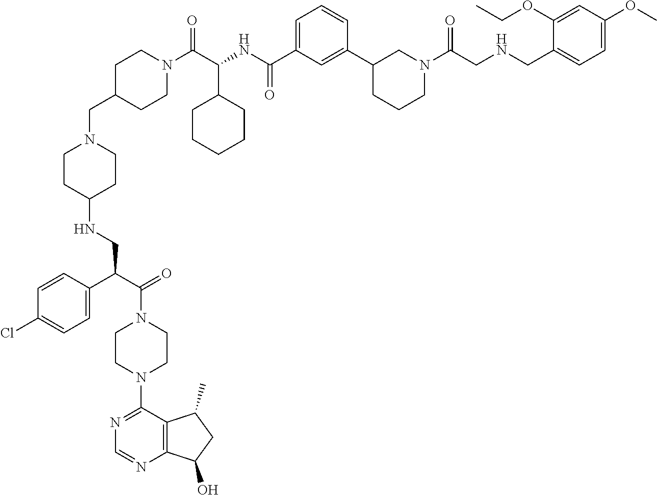 [SMILES: CCOc1cc(OC)ccc1CNCC(=O)N1CCCC(c2cccc(C(=O)N[C@@H](C(=O)N3CCC(CN4CCC(NC[C@@H](C(=O)N5CCN(c6ncnc7c6[C@H](C)C[C@H]7O)CC5)c5ccc(Cl)cc5)CC4)CC3)C3CCCCC3)c2)C1] |
| I-169 | 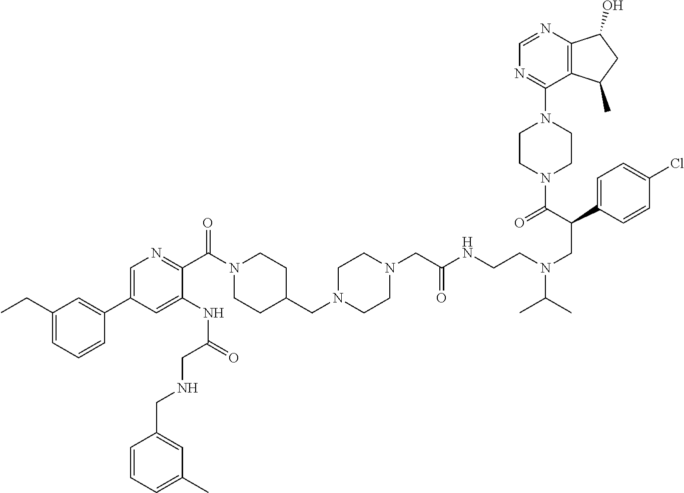 [SMILES: CCc1cccc(-c2cnc(C(=O)N3CCC(CN4CCN(CC(=O)NCCN(C[C@@H](C(=O)N5CCN(c6ncnc7c6[C@H](C)C[C@H]7O)CC5)c5ccc(Cl)cc5)C(C)C)CC4)CC3)c(NC(=O)CNCc3cccc(C)c3)c2)c1] |
| I-170 | 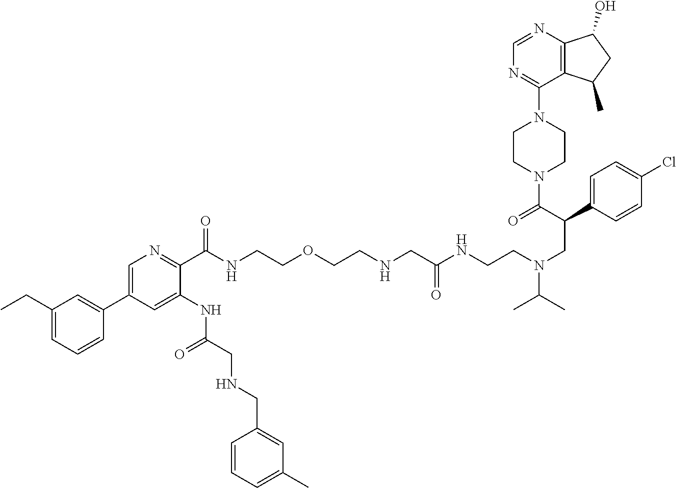 [SMILES: CCc1cccc(-c2cnc(C(=O)NCCOCCNCC(=O)NCCN(C[C@@H](C(=O)N3CCN(c4ncnc5c4[C@H](C)C[C@H]5O)CC3)c3ccc(Cl)cc3)C(C)C)c(NC(=O)CNCc3cccc(C)c3)c2)c1] |
| I-171 | 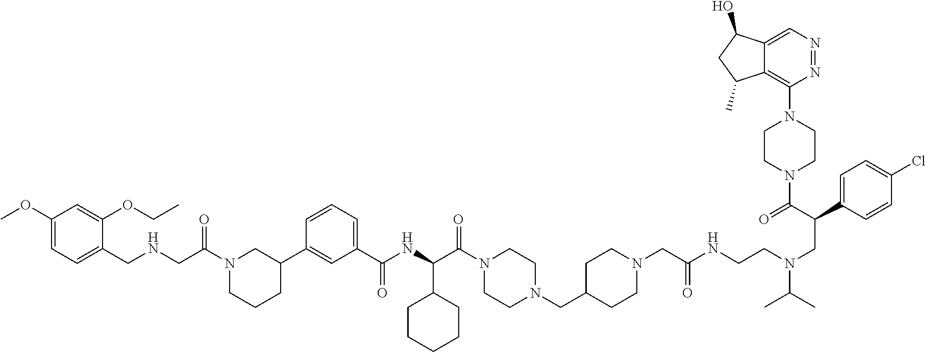 [SMILES: CCOc1cc(OC)ccc1CNCC(=O)N1CCCC(c2cccc(C(=O)N[C@@H](C(=O)N3CCN(CC4CCN(CC(=O)NCCN(C[C@@H](C(=O)N5CCN(c6nncc7c6[C@H](C)C[C@H]7O)CC5)c5ccc(Cl)cc5)C(C)C)CC4)CC3)C3CCCCC3)c2)C1] |
| I-172 | 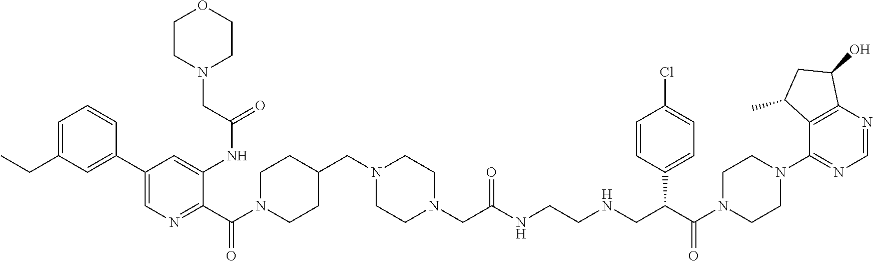 [SMILES: CCc1cccc(-c2cnc(C(=O)N3CCC(CN4CCN(CC(=O)NCCNC[C@@H](C(=O)N5CCN(c6ncnc7c6[C@H](C)C[C@H]7O)CC5)c5ccc(Cl)cc5)CC4)CC3)c(NC(=O)CN3CCOCC3)c2)c1] |
| I-173 | 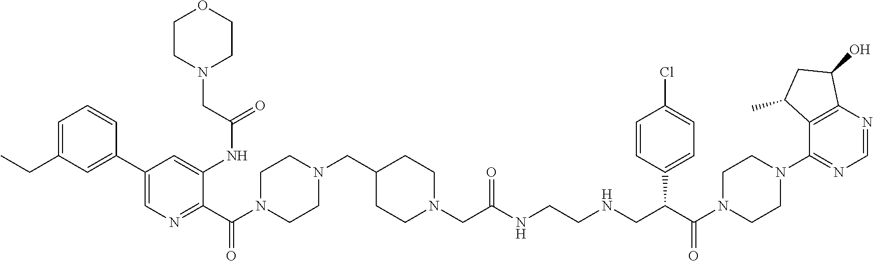 [SMILES: CCc1cccc(-c2cnc(C(=O)N3CCN(CC4CCN(CC(=O)NCCNC[C@@H](C(=O)N5CCN(c6ncnc7c6[C@H](C)C[C@H]7O)CC5)c5ccc(Cl)cc5)CC4)CC3)c(NC(=O)CN3CCOCC3)c2)c1] |
| I-174 | 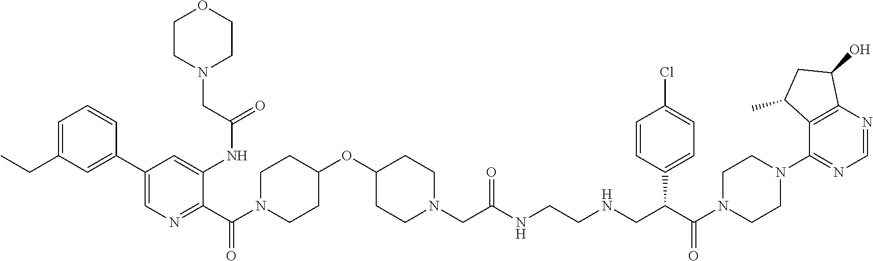 [SMILES: CCc1cccc(-c2cnc(C(=O)N3CCC(OC4CCN(CC(=O)NCCNC[C@@H](C(=O)N5CCN(c6ncnc7c6[C@H](C)C[C@H]7O)CC5)c5ccc(Cl)cc5)CC4)CC3)c(NC(=O)CN3CCOCC3)c2)c1] |
| I-175 | 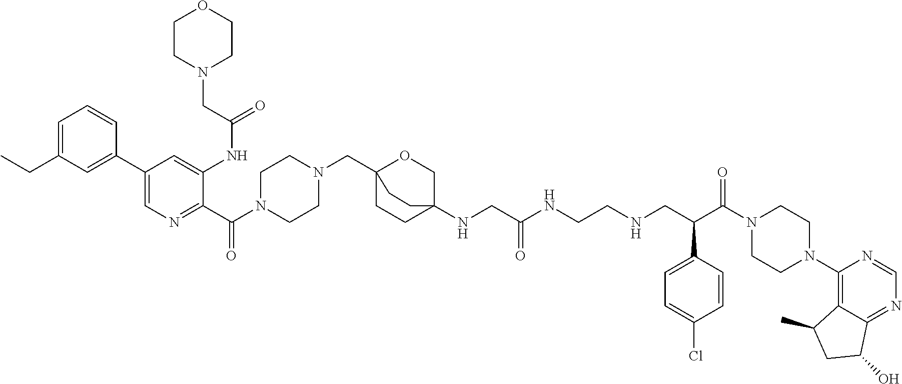 [SMILES: CCc1cccc(-c2cnc(C(=O)N3CCN(CC45CCC(NCC(=O)NCCNC[C@@H](C(=O)N6CCN(c7ncnc8c7[C@H](C)C[C@H]8O)CC6)c6ccc(Cl)cc6)(CC4)CO5)CC3)c(NC(=O)CN3CCOCC3)c2)c1] |
| I-176 | 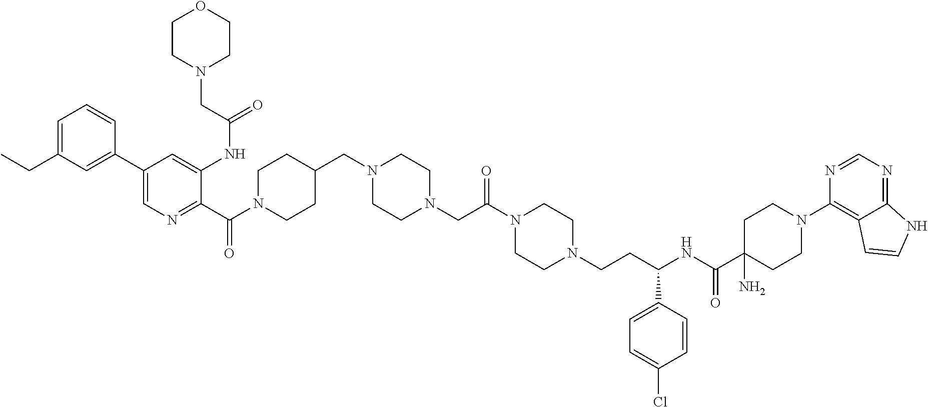 [SMILES: CCc1cccc(-c2cnc(C(=O)N3CCC(CN4CCN(CC(=O)N5CCN(CC[C@H](NC(=O)C6(N)CCN(c7ncnc8[nH]ccc78)CC6)c6ccc(Cl)cc6)CC5)CC4)CC3)c(NC(=O)CN3CCOCC3)c2)c1] |
| I-177 | 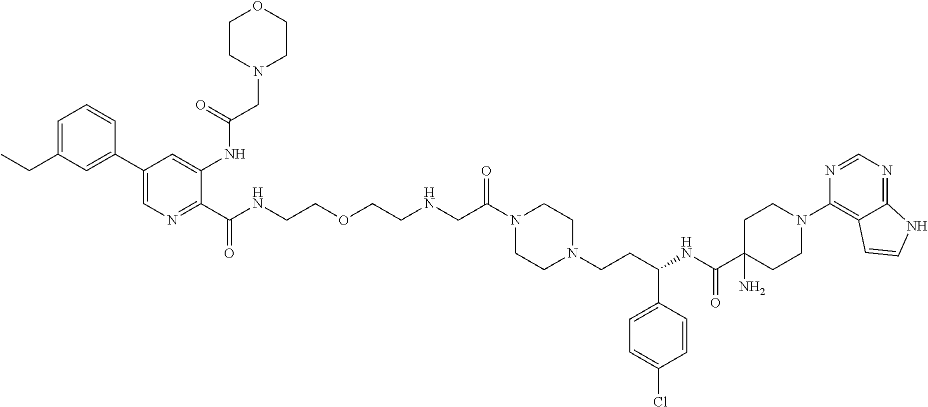 [SMILES: CCc1cccc(-c2cnc(C(=O)NCCOCCNCC(=O)N3CCN(CC[C@H](NC(=O)C4(N)CCN(c5ncnc6[nH]ccc56)CC4)c4ccc(Cl)cc4)CC3)c(NC(=O)CN3CCOCC3)c2)c1] |
| I-178 | 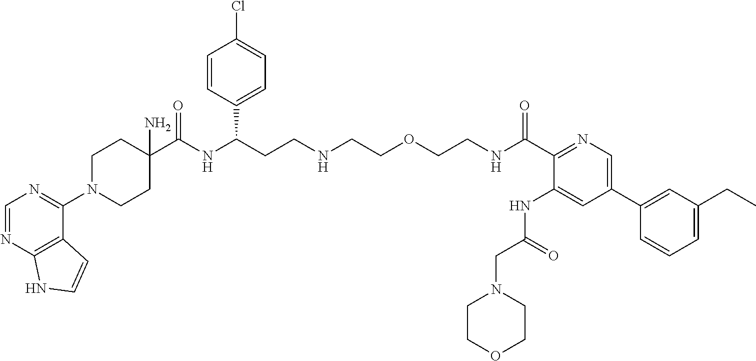 [SMILES: CCc1cccc(-c2cnc(C(=O)NCCOCCNCC[C@H](NC(=O)C3(N)CCN(c4ncnc5[nH]ccc45)CC3)c3ccc(Cl)cc3)c(NC(=O)CN3CCOCC3)c2)c1] |
| I-179 | 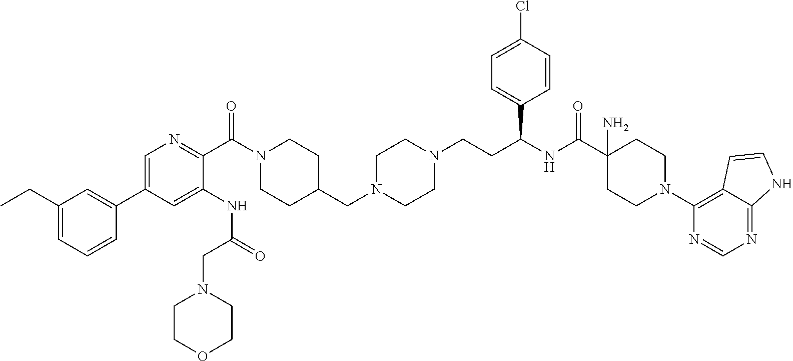 [SMILES: CCc1cccc(-c2cnc(C(=O)N3CCC(CN4CCN(CC[C@H](NC(=O)C5(N)CCN(c6ncnc7[nH]ccc67)CC5)c5ccc(Cl)cc5)CC4)CC3)c(NC(=O)CN3CCOCC3)c2)c1] |
| I-180 | 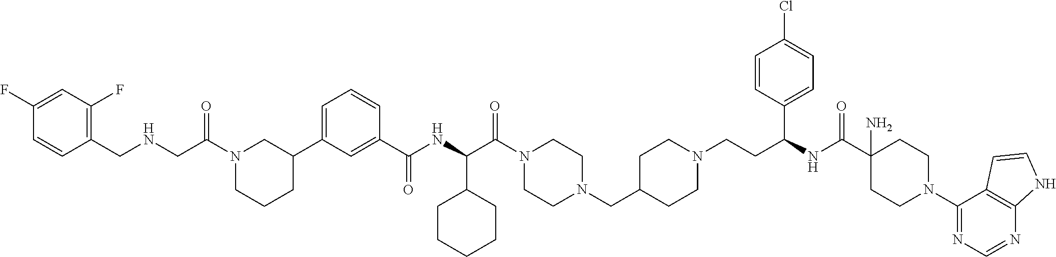 [SMILES: NC1(C(=O)N[C@@H](CCN2CCC(CN3CCN(C(=O)[C@H](NC(=O)c4cccc(C5CCCN(C(=O)CNCc6ccc(F)cc6F)C5)c4)C4CCCCC4)CC3)CC2)c2ccc(Cl)cc2)CCN(c2ncnc3[nH]ccc23)CC1] |
| I-181 | 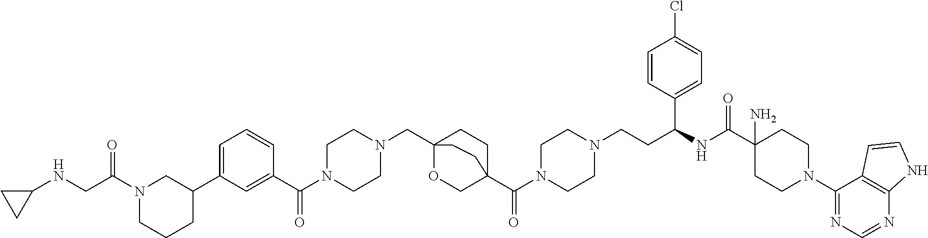 [SMILES: NC1(C(=O)N[C@@H](CCN2CCN(C(=O)C34CCC(CN5CCN(C(=O)c6cccc(C7CCCN(C(=O)CNC8CC8)C7)c6)CC5)(CC3)OC4)CC2)c2ccc(Cl)cc2)CCN(c2ncnc3[nH]ccc23)CC1] |
| I-182 | 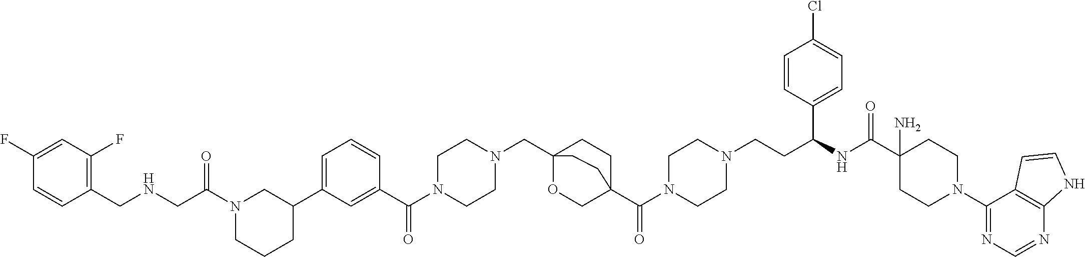 [SMILES: NC1(C(=O)N[C@@H](CCN2CCN(C(=O)C34CCC(CN5CCN(C(=O)c6cccc(C7CCCN(C(=O)CNCc8ccc(F)cc8F)C7)c6)CC5)(CC3)OC4)CC2)c2ccc(Cl)cc2)CCN(c2ncnc3[nH]ccc23)CC1] |
| I-183 | 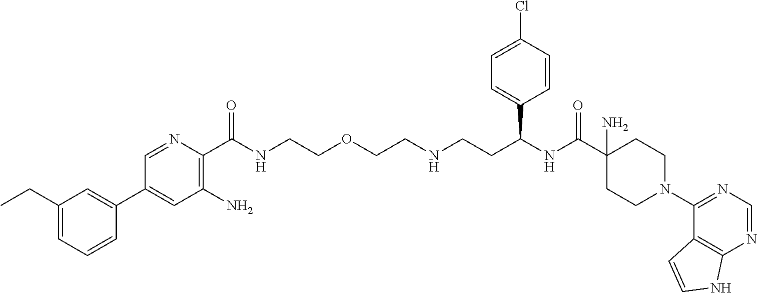 [SMILES: CCc1cccc(-c2cnc(C(=O)NCCOCCNCC[C@H](NC(=O)C3(N)CCN(c4ncnc5[nH]ccc45)CC3)c3ccc(Cl)cc3)c(N)c2)c1] |
| I-184 | 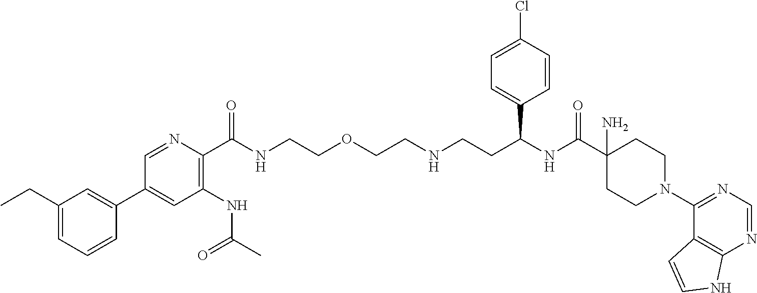 [SMILES: CCc1cccc(-c2cnc(C(=O)NCCOCCNCC[C@H](NC(=O)C3(N)CCN(c4ncnc5[nH]ccc45)CC3)c3ccc(Cl)cc3)c(NC(C)=O)c2)c1] |
| I-185 | 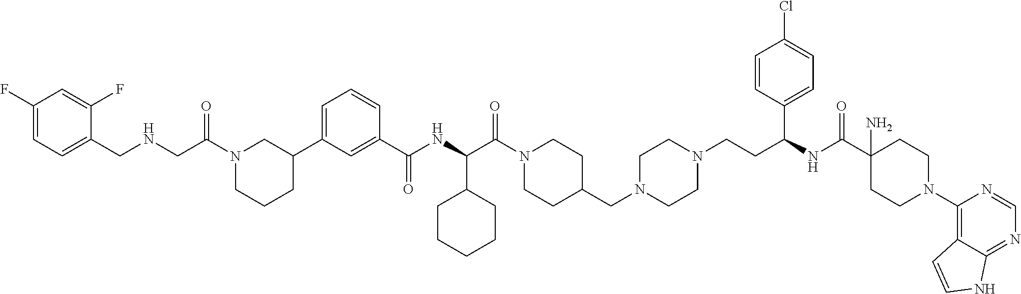 [SMILES: NC1(C(=O)N[C@@H](CCN2CCN(CC3CCN(C(=O)[C@H](NC(=O)c4cccc(C5CCCN(C(=O)CNCc6ccc(F)cc6F)C5)c4)C4CCCCC4)CC3)CC2)c2ccc(Cl)cc2)CCN(c2ncnc3[nH]ccc23)CC1] |
| I-186 | 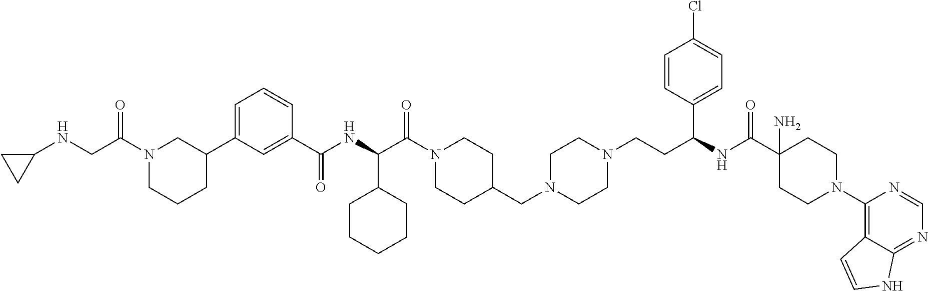 [SMILES: NC1(C(=O)N[C@@H](CCN2CCN(CC3CCN(C(=O)[C@H](NC(=O)c4cccc(C5CCCN(C(=O)CNC6CC6)C5)c4)C4CCCCC4)CC3)CC2)c2ccc(Cl)cc2)CCN(c2ncnc3[nH]ccc23)CC1] |
| I-187 | 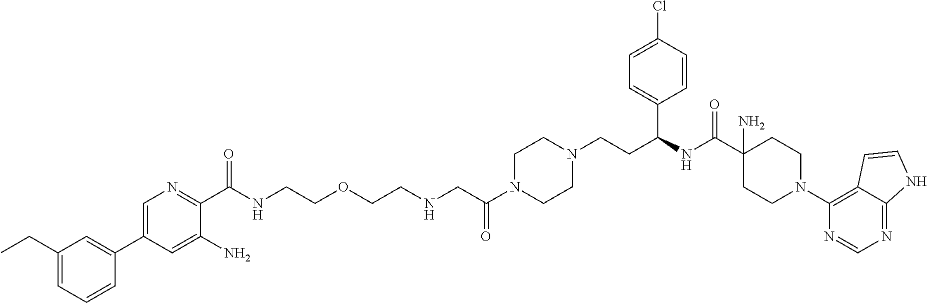 [SMILES: CCc1cccc(-c2cnc(C(=O)NCCOCCNCC(=O)N3CCN(CC[C@H](NC(=O)C4(N)CCN(c5ncnc6[nH]ccc56)CC4)c4ccc(Cl)cc4)CC3)c(N)c2)c1] |
| I-188 | 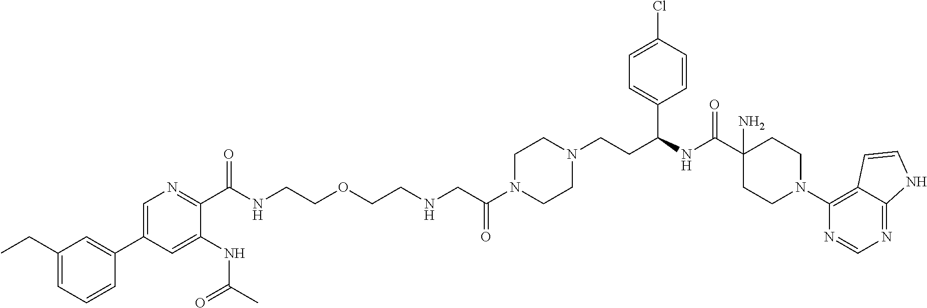 [SMILES: CCc1cccc(-c2cnc(C(=O)NCCOCCNCC(=O)N3CCN(CC[C@H](NC(=O)C4(N)CCN(c5ncnc6[nH]ccc56)CC4)c4ccc(Cl)cc4)CC3)c(NC(C)=O)c2)c1] |
| I-189 | 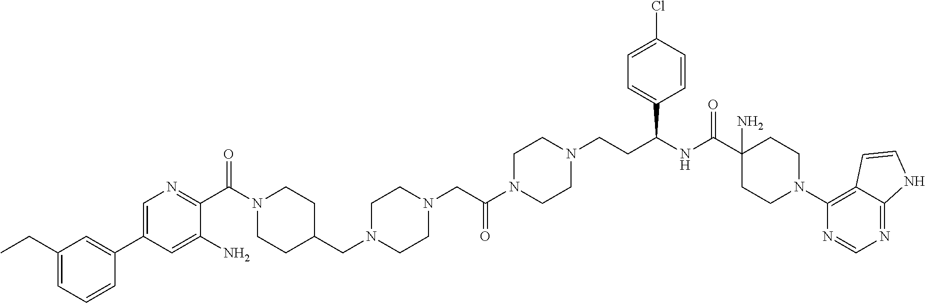 [SMILES: CCc1cccc(-c2cnc(C(=O)N3CCC(CN4CCN(CC(=O)N5CCN(CC[C@H](NC(=O)C6(N)CCN(c7ncnc8[nH]ccc78)CC6)c6ccc(Cl)cc6)CC5)CC4)CC3)c(N)c2)c1] |
| I-190 | 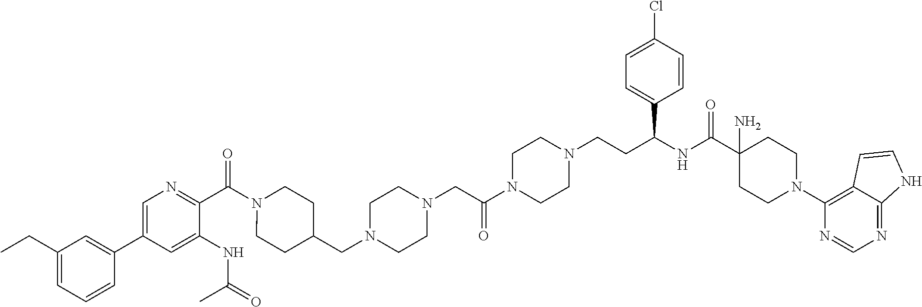 [SMILES: CCc1cccc(-c2cnc(C(=O)N3CCC(CN4CCN(CC(=O)N5CCN(CC[C@H](NC(=O)C6(N)CCN(c7ncnc8[nH]ccc78)CC6)c6ccc(Cl)cc6)CC5)CC4)CC3)c(NC(C)=O)c2)c1] |
| I-191 | 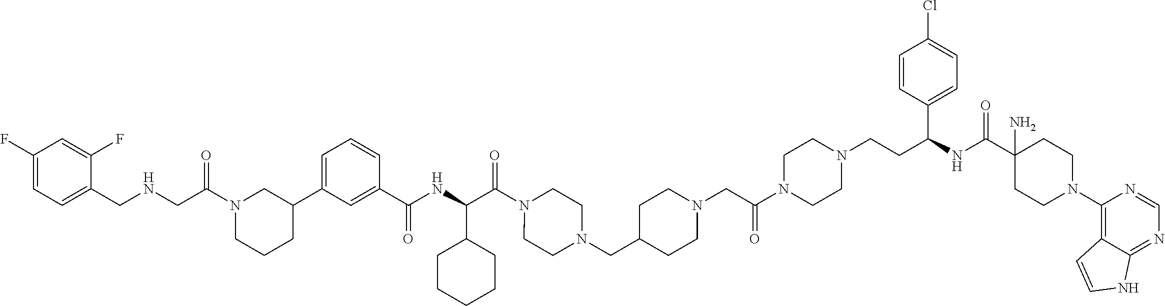 [SMILES: NC1(C(=O)N[C@@H](CCN2CCN(C(=O)CN3CCC(CN4CCN(C(=O)[C@H](NC(=O)c5cccc(C6CCCN(C(=O)CNCc7ccc(F)cc7F)C6)c5)C5CCCCC5)CC4)CC3)CC2)c2ccc(Cl)cc2)CCN(c2ncnc3[nH]ccc23)CC1] |
| I-192 | 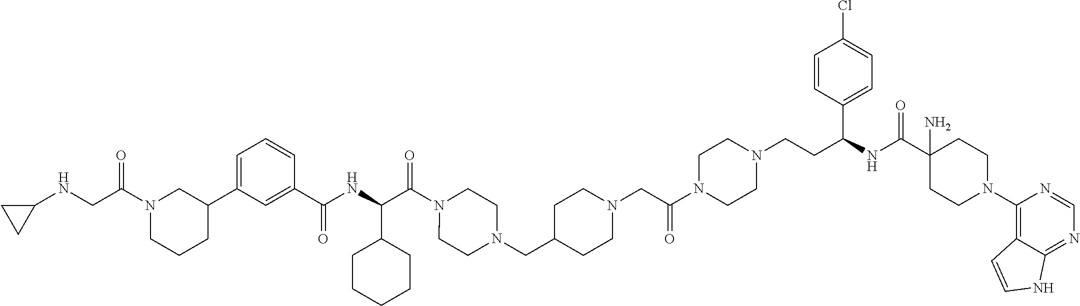 [SMILES: NC1(C(=O)N[C@@H](CCN2CCN(C(=O)CN3CCC(CN4CCN(C(=O)[C@H](NC(=O)c5cccc(C6CCCN(C(=O)CNC7CC7)C6)c5)C5CCCCC5)CC4)CC3)CC2)c2ccc(Cl)cc2)CCN(c2ncnc3[nH]ccc23)CC1] |
| I-193 | 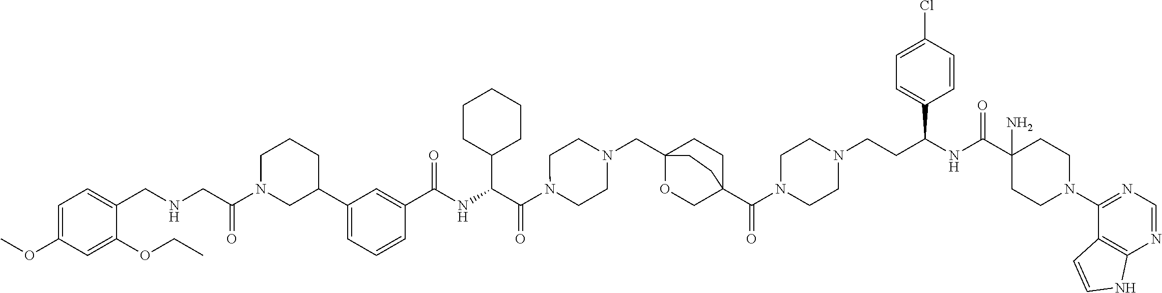 [SMILES: CCOc1cc(OC)ccc1CNCC(=O)N1CCCC(c2cccc(C(=O)N[C@@H](C(=O)N3CCN(CC45CCC(C(=O)N6CCN(CC[C@H](NC(=O)C7(N)CCN(c8ncnc9[nH]ccc89)CC7)c7ccc(Cl)cc7)CC6)(CC4)CO5)CC3)C3CCCCC3)c2)C1] |
| I-194 | 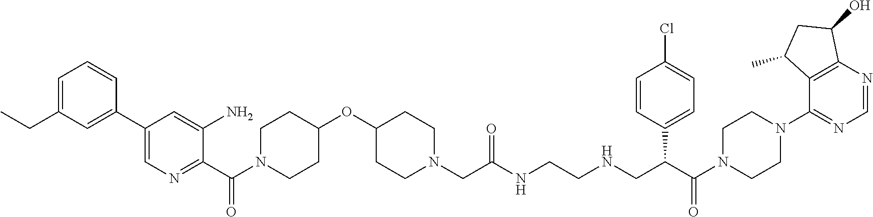 [SMILES: CCc1cccc(-c2cnc(C(=O)N3CCC(OC4CCN(CC(=O)NCCNC[C@@H](C(=O)N5CCN(c6ncnc7c6[C@H](C)C[C@H]7O)CC5)c5ccc(Cl)cc5)CC4)CC3)c(N)c2)c1] |
| I-195 | 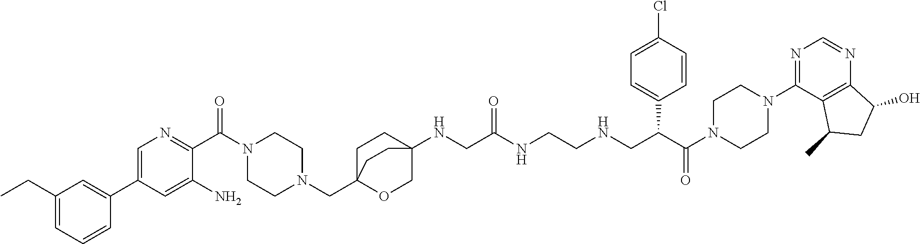 [SMILES: CCc1cccc(-c2cnc(C(=O)N3CCN(CC45CCC(NCC(=O)NCCNC[C@@H](C(=O)N6CCN(c7ncnc8c7[C@H](C)C[C@H]8O)CC6)c6ccc(Cl)cc6)(CC4)CO5)CC3)c(N)c2)c1] |
| I-196 | 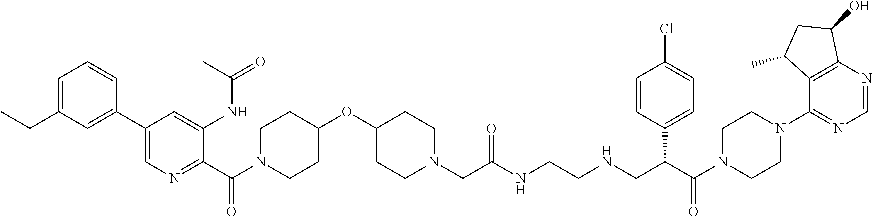 [SMILES: CCc1cccc(-c2cnc(C(=O)N3CCC(OC4CCN(CC(=O)NCCNC[C@@H](C(=O)N5CCN(c6ncnc7c6[C@H](C)C[C@H]7O)CC5)c5ccc(Cl)cc5)CC4)CC3)c(NC(C)=O)c2)c1] |
| I-197 | 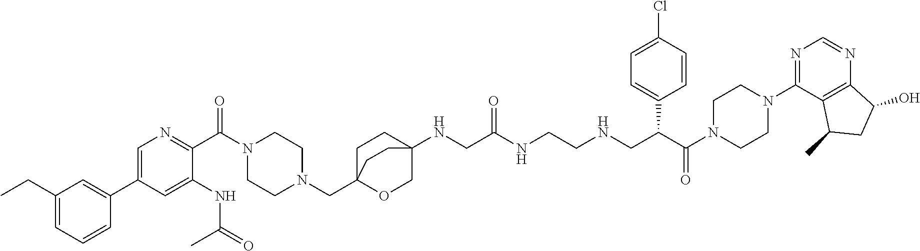 [SMILES: CCc1cccc(-c2cnc(C(=O)N3CCN(CC45CCC(NCC(=O)NCCNC[C@@H](C(=O)N6CCN(c7ncnc8c7[C@H](C)C[C@H]8O)CC6)c6ccc(Cl)cc6)(CC4)CO5)CC3)c(NC(C)=O)c2)c1] |
| I-198 | 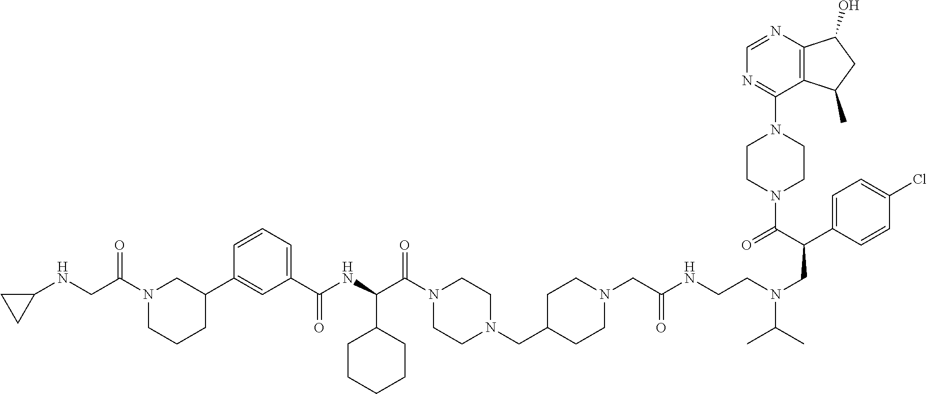 [SMILES: CC(C)N(CCNC(=O)CN1CCC(CN2CCN(C(=O)[C@H](NC(=O)c3cccc(C4CCCN(C(=O)CNC5CC5)C4)c3)C3CCCCC3)CC2)CC1)C[C@@H](C(=O)N1CCN(c2ncnc3c2[C@H](C)C[C@H]3O)CC1)c1ccc(Cl)cc1] |
| I-199 | 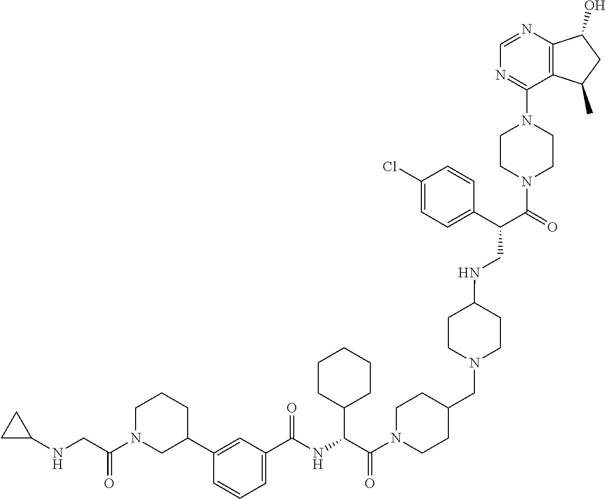 [SMILES: C[C@@H]1C[C@@H](O)c2ncnc(N3CCN(C(=O)[C@H](CNC4CCN(CC5CCN(C(=O)[C@H](NC(=O)c6cccc(C7CCCN(C(=O)CNC8CC8)C7)c6)C6CCCCC6)CC5)CC4)c4ccc(Cl)cc4)CC3)c21] |
| I-200 | 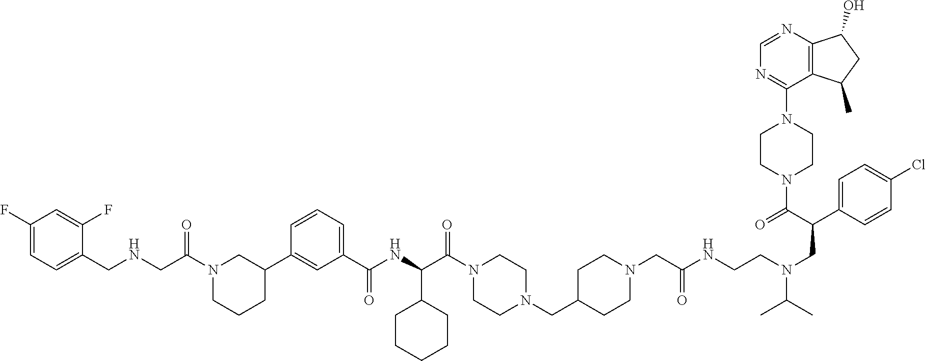 [SMILES: CC(C)N(CCNC(=O)CN1CCC(CN2CCN(C(=O)[C@H](NC(=O)c3cccc(C4CCCN(C(=O)CNCc5ccc(F)cc5F)C4)c3)C3CCCCC3)CC2)CC1)C[C@@H](C(=O)N1CCN(c2ncnc3c2[C@H](C)C[C@H]3O)CC1)c1ccc(Cl)cc1] |
| I-201 | 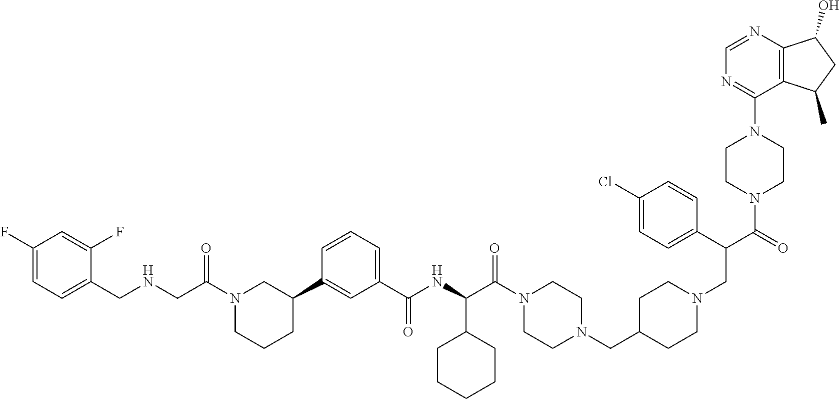 [SMILES: C[C@@H]1C[C@@H](O)c2ncnc(N3CCN(C(=O)C(CN4CCC(CN5CCN(C(=O)[C@H](NC(=O)c6cccc([C@H]7CCCN(C(=O)CNCc8ccc(F)cc8F)C7)c6)C6CCCCC6)CC5)CC4)c4ccc(Cl)cc4)CC3)c21] |
| I-202 | 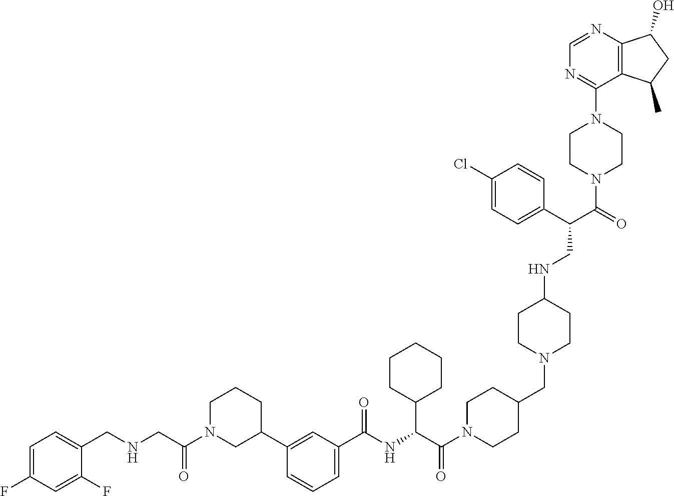 [SMILES: C[C@@H]1C[C@@H](O)c2ncnc(N3CCN(C(=O)[C@H](CNC4CCN(CC5CCN(C(=O)[C@H](NC(=O)c6cccc(C7CCCN(C(=O)CNCc8ccc(F)cc8F)C7)c6)C6CCCCC6)CC5)CC4)c4ccc(Cl)cc4)CC3)c21] |
| I-203 | 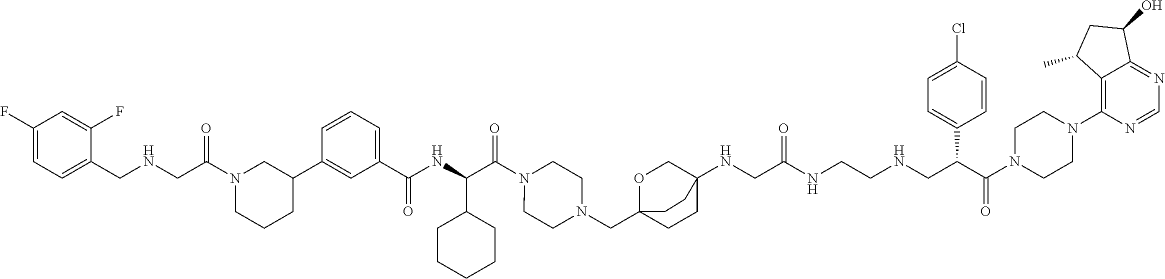 [SMILES: C[C@@H]1C[C@@H](O)c2ncnc(N3CCN(C(=O)[C@H](CNCCNC(=O)CNC45CCC(CN6CCN(C(=O)[C@H](NC(=O)c7cccc(C8CCCN(C(=O)CNCc9ccc(F)cc9F)C8)c7)C7CCCCC7)CC6)(CC4)OC5)c4ccc(Cl)cc4)CC3)c21] |
| I-204 | 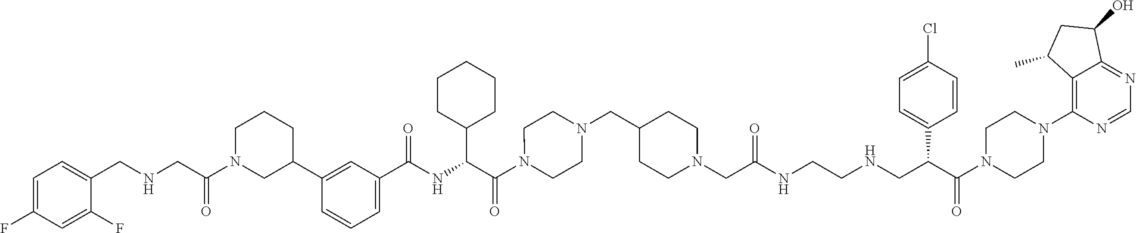 [SMILES: C[C@@H]1C[C@@H](O)c2ncnc(N3CCN(C(=O)[C@H](CNCCNC(=O)CN4CCC(CN5CCN(C(=O)[C@H](NC(=O)c6cccc(C7CCCN(C(=O)CNCc8ccc(F)cc8F)C7)c6)C6CCCCC6)CC5)CC4)c4ccc(Cl)cc4)CC3)c21] |
| I-205 | 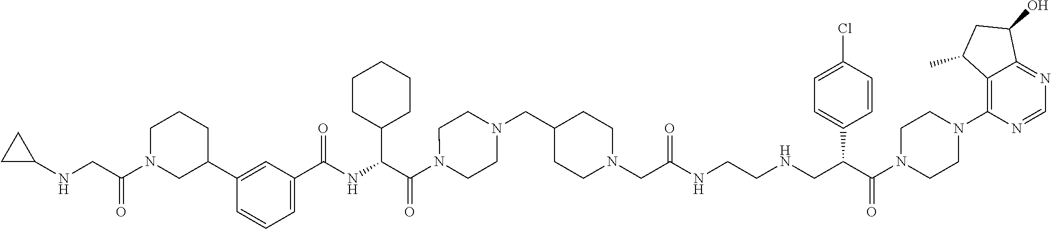 [SMILES: C[C@@H]1C[C@@H](O)c2ncnc(N3CCN(C(=O)[C@H](CNCCNC(=O)CN4CCC(CN5CCN(C(=O)[C@H](NC(=O)c6cccc(C7CCCN(C(=O)CNC8CC8)C7)c6)C6CCCCC6)CC5)CC4)c4ccc(Cl)cc4)CC3)c21] |
| I-206 | 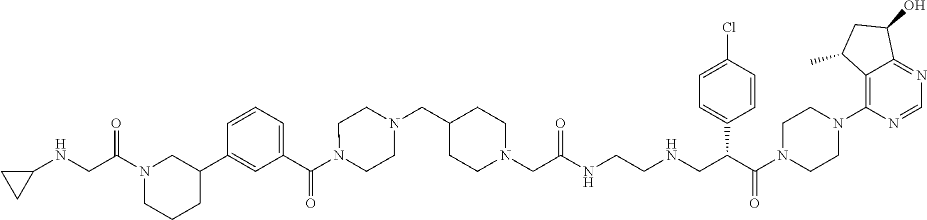 [SMILES: C[C@@H]1C[C@@H](O)c2ncnc(N3CCN(C(=O)[C@H](CNCCNC(=O)CN4CCC(CN5CCN(C(=O)c6cccc(C7CCCN(C(=O)CNC8CC8)C7)c6)CC5)CC4)c4ccc(Cl)cc4)CC3)c21] |
| I-207 | 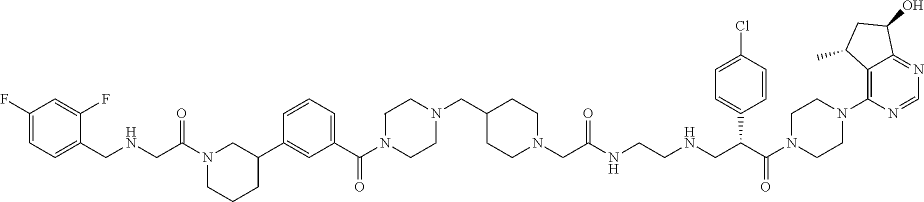 [SMILES: C[C@@H]1C[C@@H](O)c2ncnc(N3CCN(C(=O)[C@H](CNCCNC(=O)CN4CCC(CN5CCN(C(=O)c6cccc(C7CCCN(C(=O)CNCc8ccc(F)cc8F)C7)c6)CC5)CC4)c4ccc(Cl)cc4)CC3)c21] |
| I-208 | 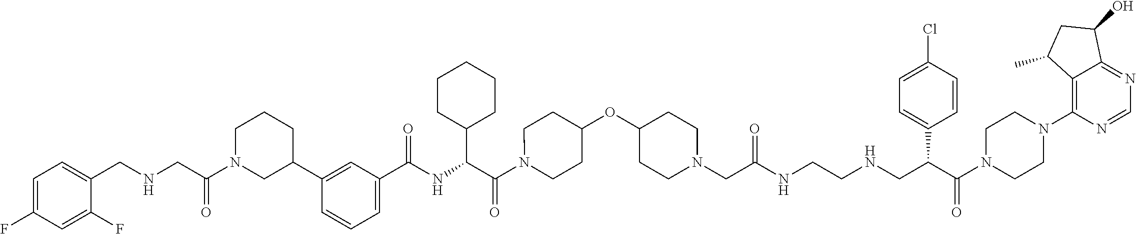 [SMILES: C[C@@H]1C[C@@H](O)c2ncnc(N3CCN(C(=O)[C@H](CNCCNC(=O)CN4CCC(OC5CCN(C(=O)[C@H](NC(=O)c6cccc(C7CCCN(C(=O)CNCc8ccc(F)cc8F)C7)c6)C6CCCCC6)CC5)CC4)c4ccc(Cl)cc4)CC3)c21] |
| I-209 | 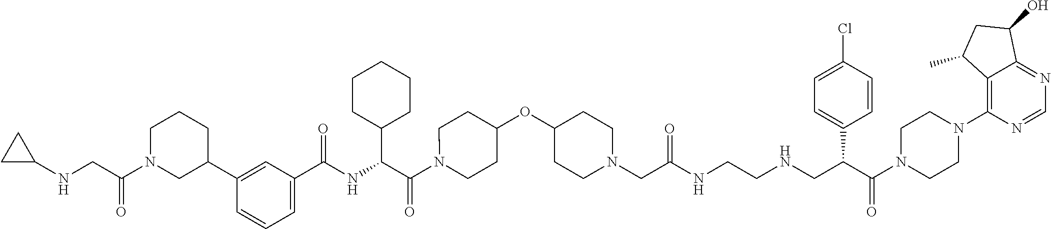 [SMILES: C[C@@H]1C[C@@H](O)c2ncnc(N3CCN(C(=O)[C@H](CNCCNC(=O)CN4CCC(OC5CCN(C(=O)[C@H](NC(=O)c6cccc(C7CCCN(C(=O)CNC8CC8)C7)c6)C6CCCCC6)CC5)CC4)c4ccc(Cl)cc4)CC3)c21] |
| I-210 | 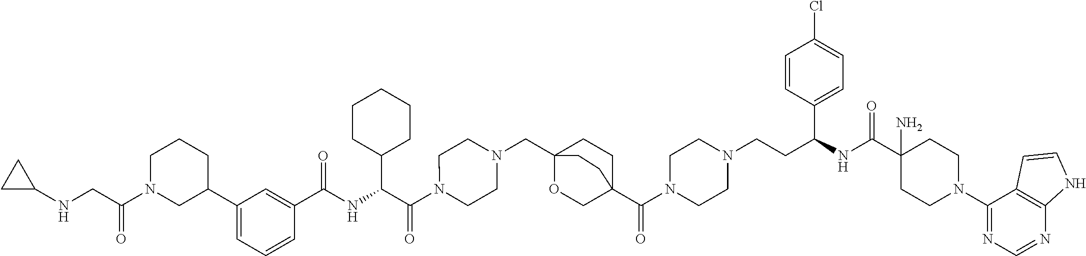 [SMILES: NC1(C(=O)N[C@@H](CCN2CCN(C(=O)C34CCC(CN5CCN(C(=O)[C@H](NC(=O)c6cccc(C7CCCN(C(=O)CNC8CC8)C7)c6)C6CCCCC6)CC5)(CC3)OC4)CC2)c2ccc(Cl)cc2)CCN(c2ncnc3[nH]ccc23)CC1] |
| I-211 | 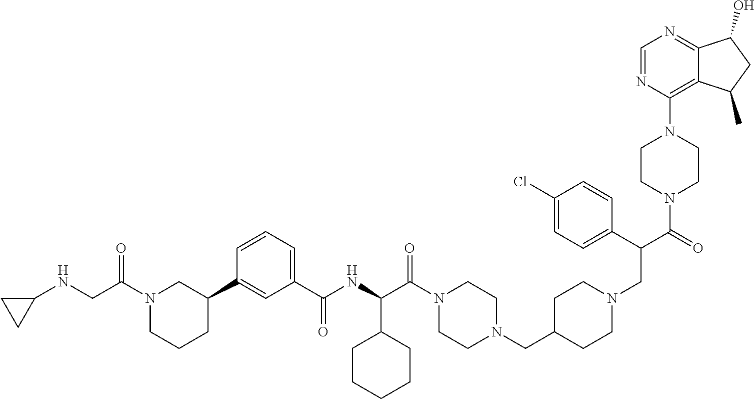 [SMILES: C[C@@H]1C[C@@H](O)c2ncnc(N3CCN(C(=O)C(CN4CCC(CN5CCN(C(=O)[C@H](NC(=O)c6cccc([C@H]7CCCN(C(=O)CNC8CC8)C7)c6)C6CCCCC6)CC5)CC4)c4ccc(Cl)cc4)CC3)c21] |
| I-212 | 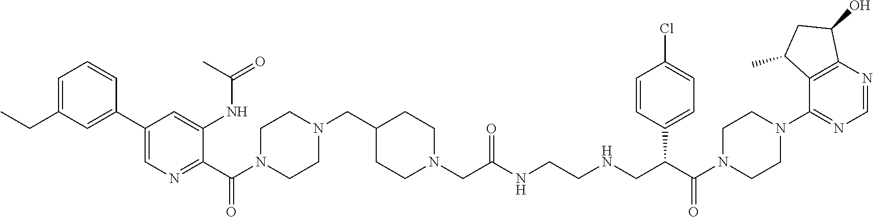 [SMILES: CCc1cccc(-c2cnc(C(=O)N3CCN(CC4CCN(CC(=O)NCCNC[C@@H](C(=O)N5CCN(c6ncnc7c6[C@H](C)C[C@H]7O)CC5)c5ccc(Cl)cc5)CC4)CC3)c(NC(C)=O)c2)c1] |
| I-213 | 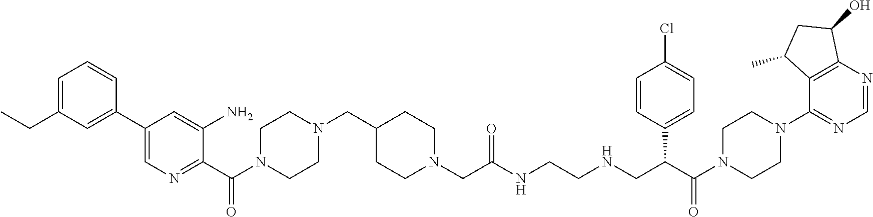 [SMILES: CCc1cccc(-c2cnc(C(=O)N3CCN(CC4CCN(CC(=O)NCCNC[C@@H](C(=O)N5CCN(c6ncnc7c6[C@H](C)C[C@H]7O)CC5)c5ccc(Cl)cc5)CC4)CC3)c(N)c2)c1] |
| I-214 | 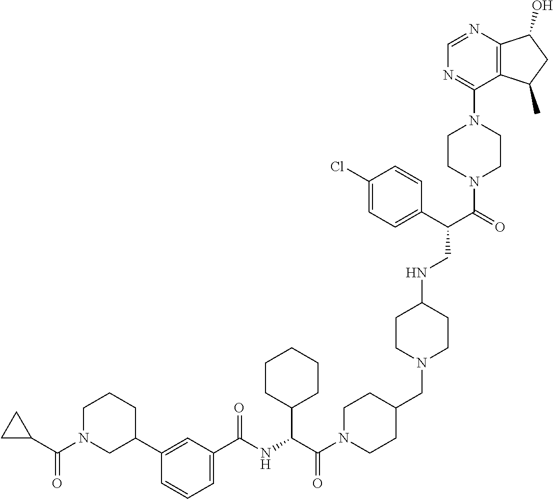 [SMILES: C[C@@H]1C[C@@H](O)c2ncnc(N3CCN(C(=O)[C@H](CNC4CCN(CC5CCN(C(=O)[C@H](NC(=O)c6cccc(C7CCCN(C(=O)C8CC8)C7)c6)C6CCCCC6)CC5)CC4)c4ccc(Cl)cc4)CC3)c21] |
| I-215 | 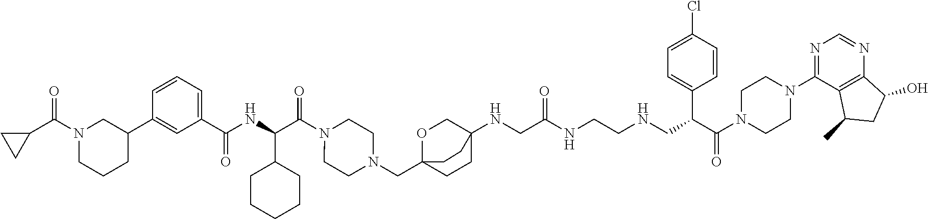 [SMILES: C[C@@H]1C[C@@H](O)c2ncnc(N3CCN(C(=O)[C@H](CNCCNC(=O)CNC45CCC(CN6CCN(C(=O)[C@H](NC(=O)c7cccc(C8CCCN(C(=O)C9CC9)C8)c7)C7CCCCC7)CC6)(CC4)OC5)c4ccc(Cl)cc4)CC3)c21] |
| I-216 | 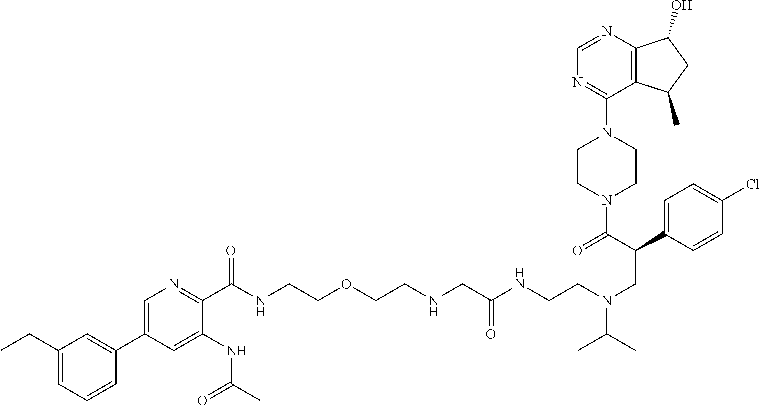 [SMILES: CCc1cccc(-c2cnc(C(=O)NCCOCCNCC(=O)NCCN(C[C@@H](C(=O)N3CCN(c4ncnc5c4[C@H](C)C[C@H]5O)CC3)c3ccc(Cl)cc3)C(C)C)c(NC(C)=O)c2)c1] |
| I-217 | 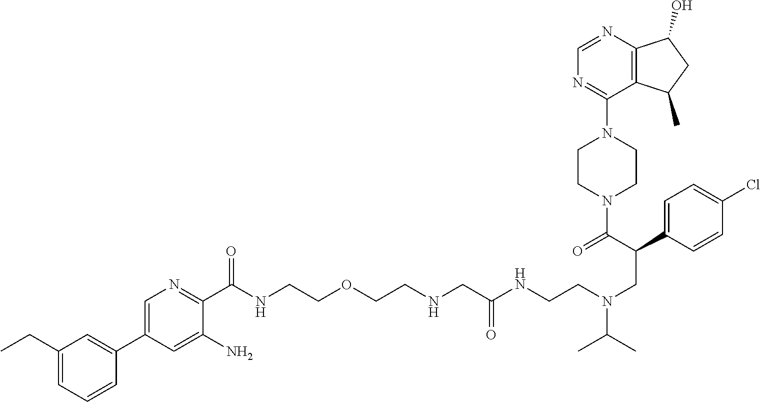 [SMILES: CCc1cccc(-c2cnc(C(=O)NCCOCCNCC(=O)NCCN(C[C@@H](C(=O)N3CCN(c4ncnc5c4[C@H](C)C[C@H]5O)CC3)c3ccc(Cl)cc3)C(C)C)c(N)c2)c1] |
| I-218 | 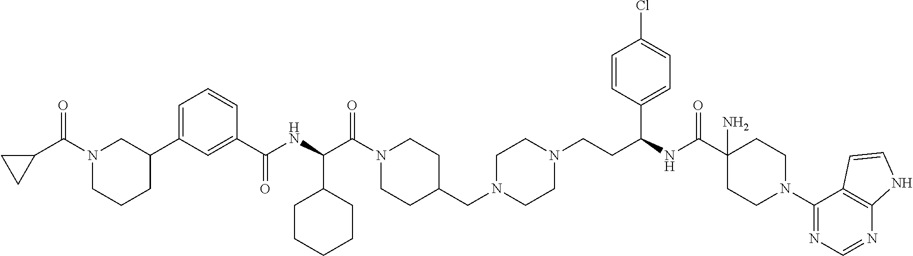 [SMILES: NC1(C(=O)N[C@@H](CCN2CCN(CC3CCN(C(=O)[C@H](NC(=O)c4cccc(C5CCCN(C(=O)C6CC6)C5)c4)C4CCCCC4)CC3)CC2)c2ccc(Cl)cc2)CCN(c2ncnc3[nH]ccc23)CC1] |
| I-219 | 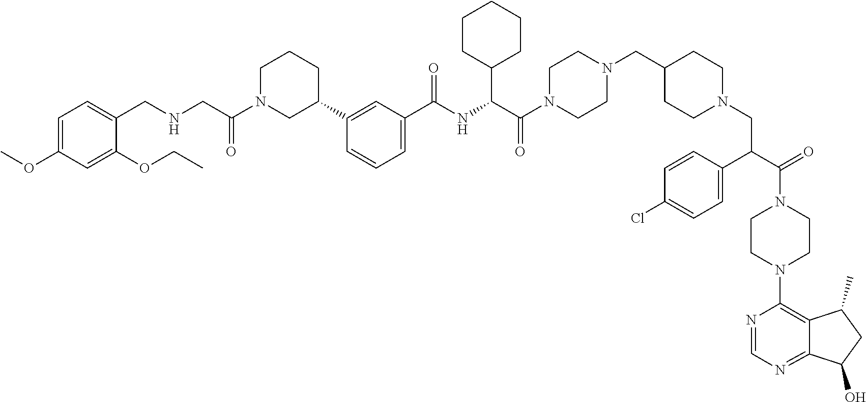 [SMILES: CCOc1cc(OC)ccc1CNCC(=O)N1CCC[C@H](c2cccc(C(=O)N[C@@H](C(=O)N3CCN(CC4CCN(CC(C(=O)N5CCN(c6ncnc7c6[C@H](C)C[C@H]7O)CC5)c5ccc(Cl)cc5)CC4)CC3)C3CCCCC3)c2)C1] |
| I-220 | 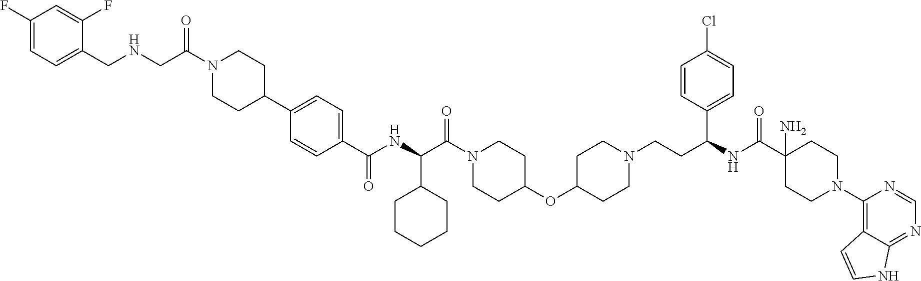 [SMILES: NC1(C(=O)N[C@@H](CCN2CCC(OC3CCN(C(=O)[C@H](NC(=O)c4ccc(C5CCN(C(=O)CNCc6ccc(F)cc6F)CC5)cc4)C4CCCCC4)CC3)CC2)c2ccc(Cl)cc2)CCN(c2ncnc3[nH]ccc23)CC1] |
| I-221 | 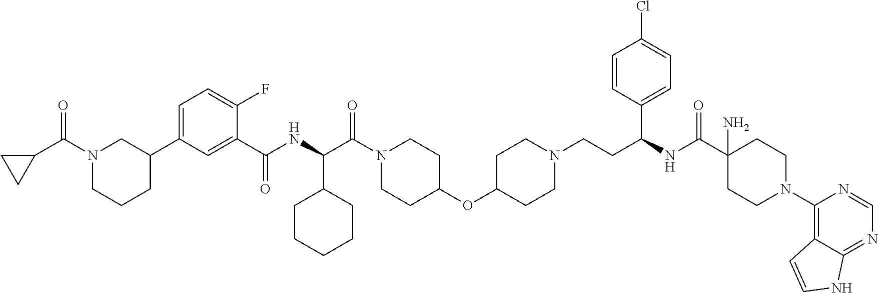 [SMILES: NC1(C(=O)N[C@@H](CCN2CCC(OC3CCN(C(=O)[C@H](NC(=O)c4cc(C5CCCN(C(=O)C6CC6)C5)ccc4F)C4CCCCC4)CC3)CC2)c2ccc(Cl)cc2)CCN(c2ncnc3[nH]ccc23)CC1] |
| I-222 | 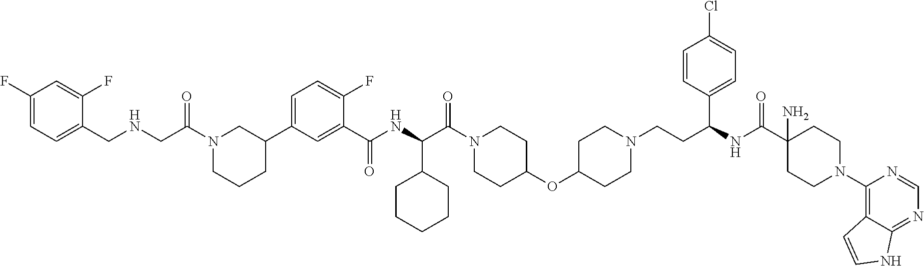 [SMILES: NC1(C(=O)N[C@@H](CCN2CCC(OC3CCN(C(=O)[C@H](NC(=O)c4cc(C5CCCN(C(=O)CNCc6ccc(F)cc6F)C5)ccc4F)C4CCCCC4)CC3)CC2)c2ccc(Cl)cc2)CCN(c2ncnc3[nH]ccc23)CC1] |
| I-223 | 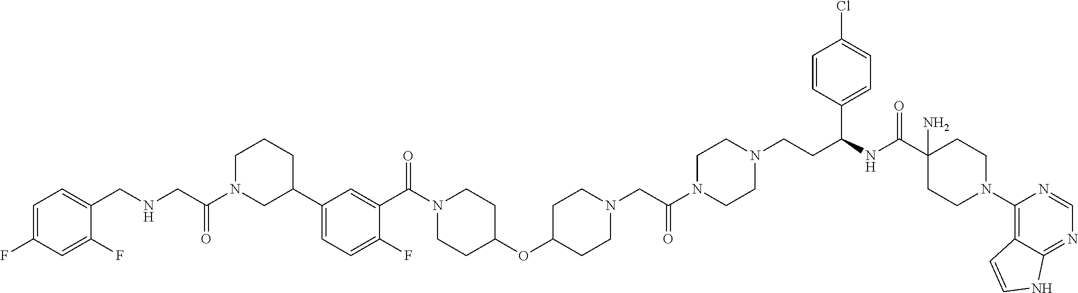 [SMILES: NC1(C(=O)N[C@@H](CCN2CCN(C(=O)CN3CCC(OC4CCN(C(=O)c5cc(C6CCCN(C(=O)CNCc7ccc(F)cc7F)C6)ccc5F)CC4)CC3)CC2)c2ccc(Cl)cc2)CCN(c2ncnc3[nH]ccc23)CC1] |
| I-224 | 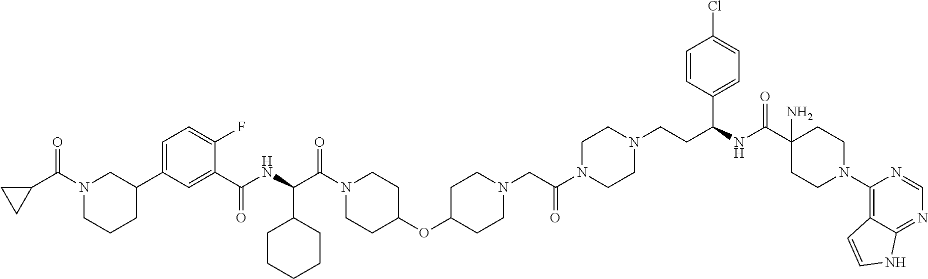 [SMILES: NC1(C(=O)N[C@@H](CCN2CCN(C(=O)CN3CCC(OC4CCN(C(=O)[C@H](NC(=O)c5cc(C6CCCN(C(=O)C7CC7)C6)ccc5F)C5CCCCC5)CC4)CC3)CC2)c2ccc(Cl)cc2)CCN(c2ncnc3[nH]ccc23)CC1] |
| I-225 | 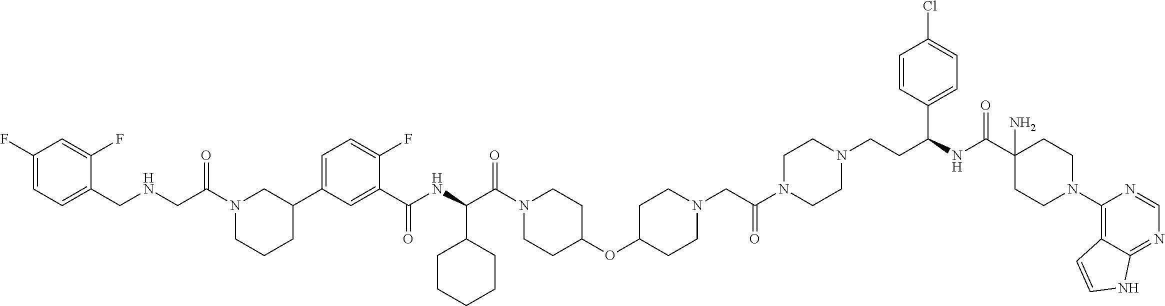 [SMILES: NC1(C(=O)N[C@@H](CCN2CCN(C(=O)CN3CCC(OC4CCN(C(=O)[C@H](NC(=O)c5cc(C6CCCN(C(=O)CNCc7ccc(F)cc7F)C6)ccc5F)C5CCCCC5)CC4)CC3)CC2)c2ccc(Cl)cc2)CCN(c2ncnc3[nH]ccc23)CC1] |
| I-226 | 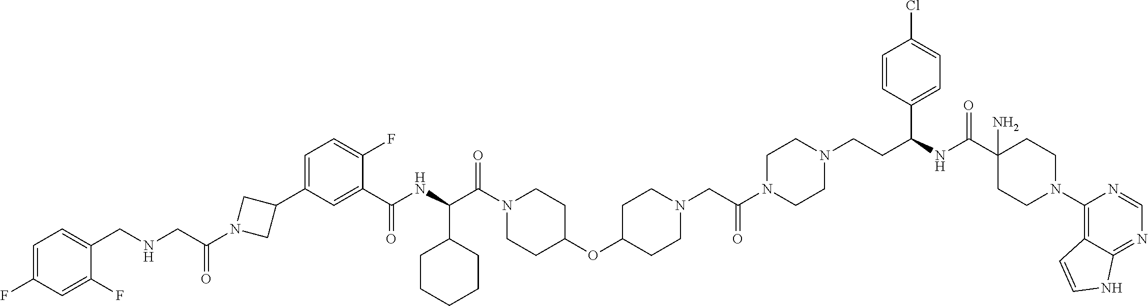 [SMILES: NC1(C(=O)N[C@@H](CCN2CCN(C(=O)CN3CCC(OC4CCN(C(=O)[C@H](NC(=O)c5cc(C6CN(C(=O)CNCc7ccc(F)cc7F)C6)ccc5F)C5CCCCC5)CC4)CC3)CC2)c2ccc(Cl)cc2)CCN(c2ncnc3[nH]ccc23)CC1] |
| I-227 | 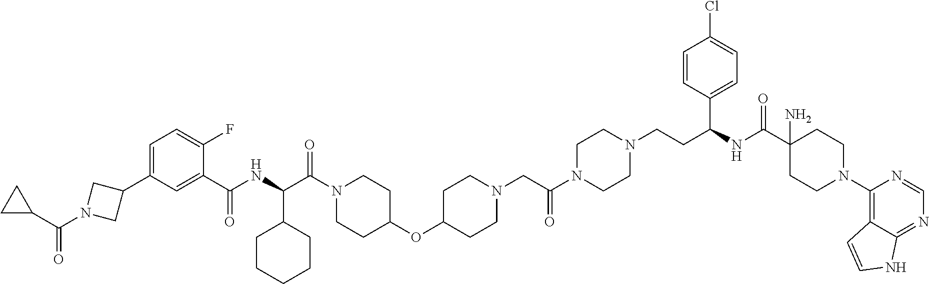 [SMILES: NC1(C(=O)N[C@@H](CCN2CCN(C(=O)CN3CCC(OC4CCN(C(=O)[C@H](NC(=O)c5cc(C6CN(C(=O)C7CC7)C6)ccc5F)C5CCCCC5)CC4)CC3)CC2)c2ccc(Cl)cc2)CCN(c2ncnc3[nH]ccc23)CC1] |
| I-228 | 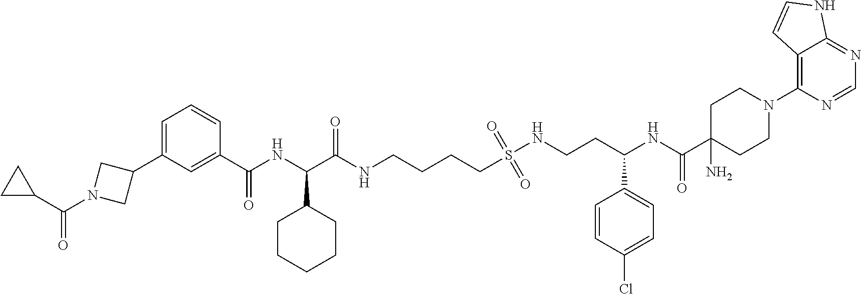 [SMILES: NC1(C(=O)N[C@@H](CCNS(=O)(=O)CCCCNC(=O)[C@H](NC(=O)c2cccc(C3CN(C(=O)C4CC4)C3)c2)C2CCCCC2)c2ccc(Cl)cc2)CCN(c2ncnc3[nH]ccc23)CC1] |
| I-229 | 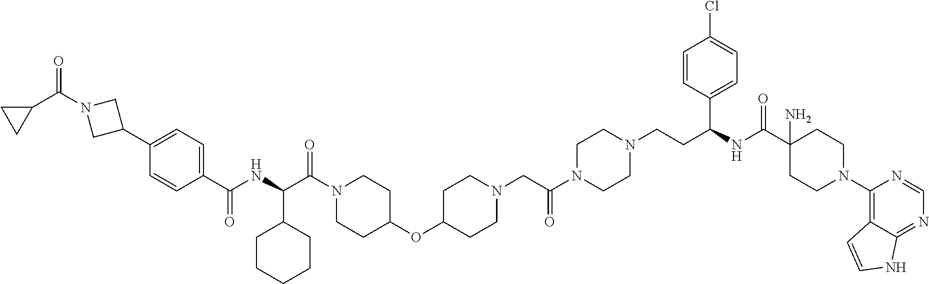 [SMILES: NC1(C(=O)N[C@@H](CCN2CCN(C(=O)CN3CCC(OC4CCN(C(=O)[C@H](NC(=O)c5ccc(C6CN(C(=O)C7CC7)C6)cc5)C5CCCCC5)CC4)CC3)CC2)c2ccc(Cl)cc2)CCN(c2ncnc3[nH]ccc23)CC1] |
| I-230 | 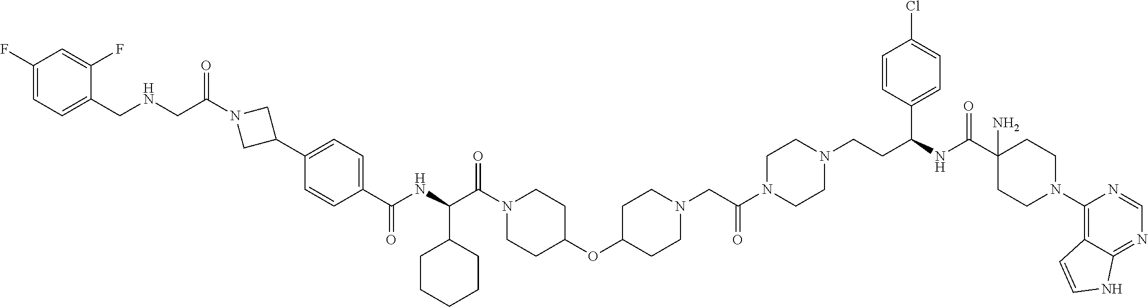 [SMILES: NC1(C(=O)N[C@@H](CCN2CCN(C(=O)CN3CCC(OC4CCN(C(=O)[C@H](NC(=O)c5ccc(C6CN(C(=O)CNCc7ccc(F)cc7F)C6)cc5)C5CCCCC5)CC4)CC3)CC2)c2ccc(Cl)cc2)CCN(c2ncnc3[nH]ccc23)CC1] |
| I-231 | 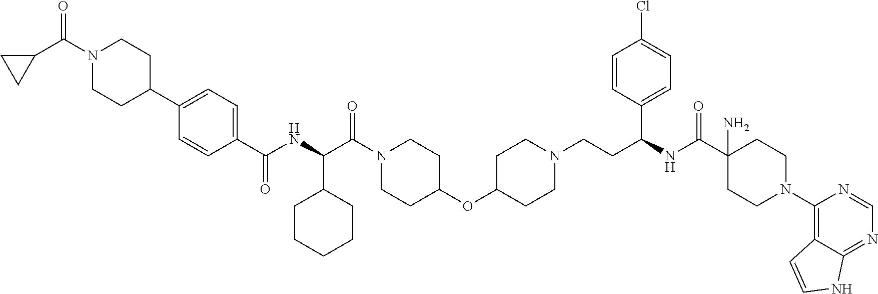 [SMILES: NC1(C(=O)N[C@@H](CCN2CCC(OC3CCN(C(=O)[C@H](NC(=O)c4ccc(C5CCN(C(=O)C6CC6)CC5)cc4)C4CCCCC4)CC3)CC2)c2ccc(Cl)cc2)CCN(c2ncnc3[nH]ccc23)CC1] |
| I-232 | 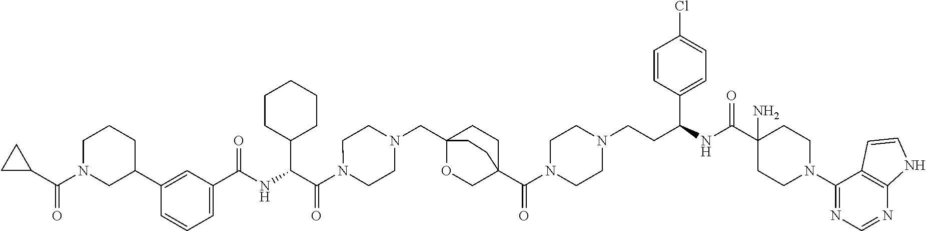 [SMILES: NC1(C(=O)N[C@@H](CCN2CCN(C(=O)C34CCC(CN5CCN(C(=O)[C@H](NC(=O)c6cccc(C7CCCN(C(=O)C8CC8)C7)c6)C6CCCCC6)CC5)(CC3)OC4)CC2)c2ccc(Cl)cc2)CCN(c2ncnc3[nH]ccc23)CC1] |
| I-233 | 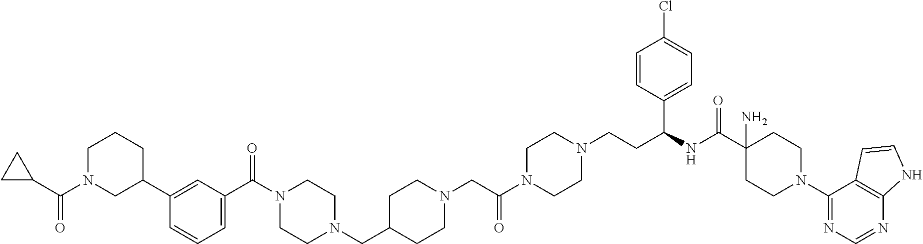 [SMILES: NC1(C(=O)N[C@@H](CCN2CCN(C(=O)CN3CCC(CN4CCN(C(=O)c5cccc(C6CCCN(C(=O)C7CC7)C6)c5)CC4)CC3)CC2)c2ccc(Cl)cc2)CCN(c2ncnc3[nH]ccc23)CC1] |
| I-234 | 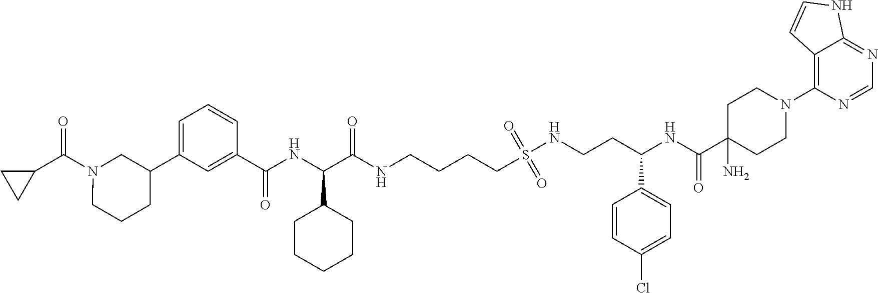 [SMILES: NC1(C(=O)N[C@@H](CCNS(=O)(=O)CCCCNC(=O)[C@H](NC(=O)c2cccc(C3CCCN(C(=O)C4CC4)C3)c2)C2CCCCC2)c2ccc(Cl)cc2)CCN(c2ncnc3[nH]ccc23)CC1] |
| I-235 | 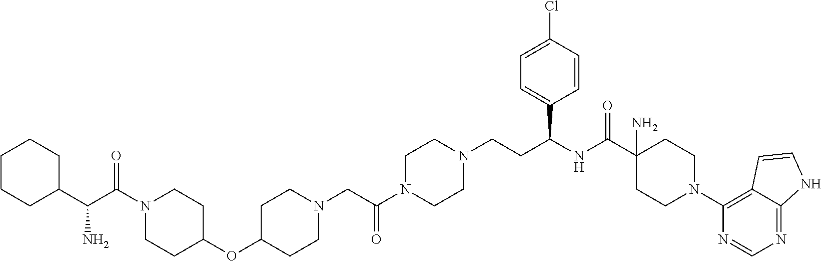 [SMILES: N[C@@H](C(=O)N1CCC(OC2CCN(CC(=O)N3CCN(CC[C@H](NC(=O)C4(N)CCN(c5ncnc6[nH]ccc56)CC4)c4ccc(Cl)cc4)CC3)CC2)CC1)C1CCCCC1] |
| I-236 | 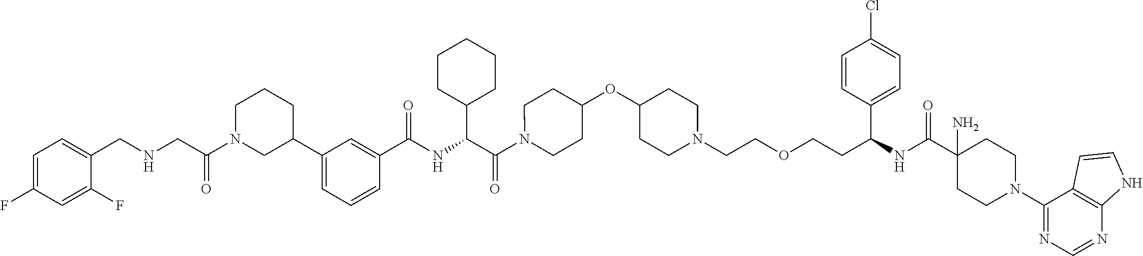 [SMILES: NC1(C(=O)N[C@@H](CCOCCN2CCC(OC3CCN(C(=O)[C@H](NC(=O)c4cccc(C5CCCN(C(=O)CNCc6ccc(F)cc6F)C5)c4)C4CCCCC4)CC3)CC2)c2ccc(Cl)cc2)CCN(c2ncnc3[nH]ccc23)CC1] |
| I-237 | 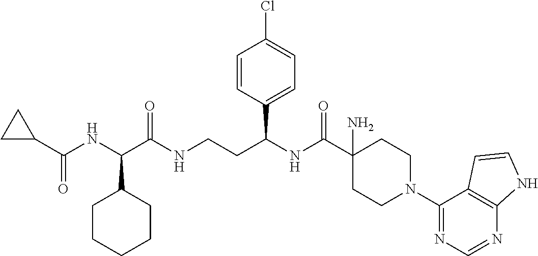 [SMILES: NC1(C(=O)N[C@@H](CCNC(=O)[C@H](NC(=O)C2CC2)C2CCCCC2)c2ccc(Cl)cc2)CCN(c2ncnc3[nH]ccc23)CC1] |
| I-238 | 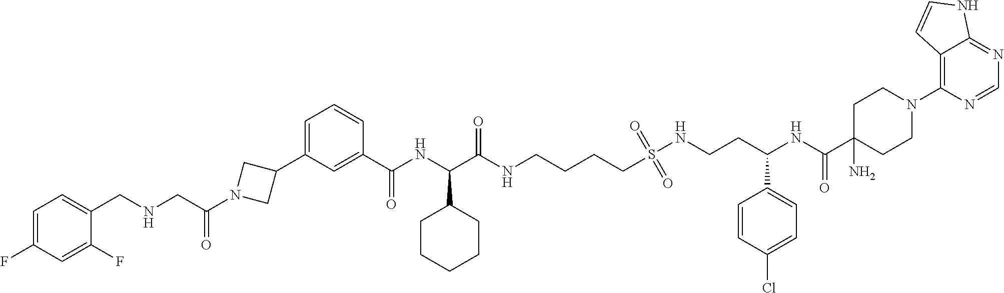 [SMILES: NC1(C(=O)N[C@@H](CCNS(=O)(=O)CCCCNC(=O)[C@H](NC(=O)c2cccc(C3CN(C(=O)CNCc4ccc(F)cc4F)C3)c2)C2CCCCC2)c2ccc(Cl)cc2)CCN(c2ncnc3[nH]ccc23)CC1] |
| I-239 | 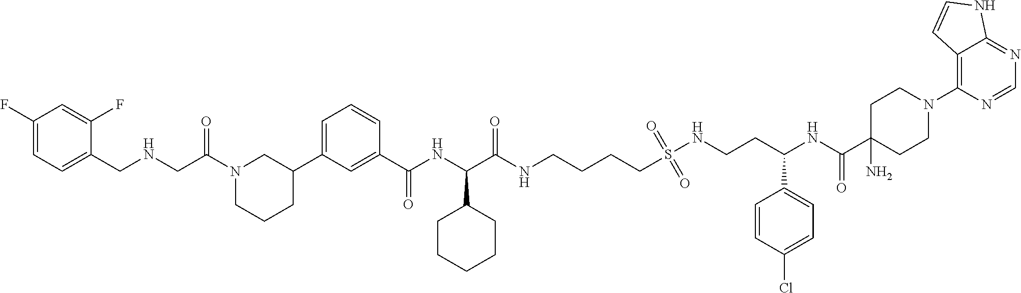 [SMILES: NC1(C(=O)N[C@@H](CCNS(=O)(=O)CCCCNC(=O)[C@H](NC(=O)c2cccc(C3CCCN(C(=O)CNCc4ccc(F)cc4F)C3)c2)C2CCCCC2)c2ccc(Cl)cc2)CCN(c2ncnc3[nH]ccc23)CC1] |
| I-240 | 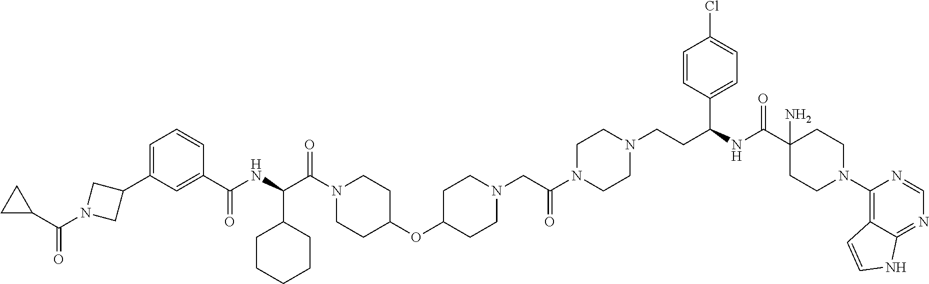 [SMILES: NC1(C(=O)N[C@@H](CCN2CCN(C(=O)CN3CCC(OC4CCN(C(=O)[C@H](NC(=O)c5cccc(C6CN(C(=O)C7CC7)C6)c5)C5CCCCC5)CC4)CC3)CC2)c2ccc(Cl)cc2)CCN(c2ncnc3[nH]ccc23)CC1] |
| I-241 | 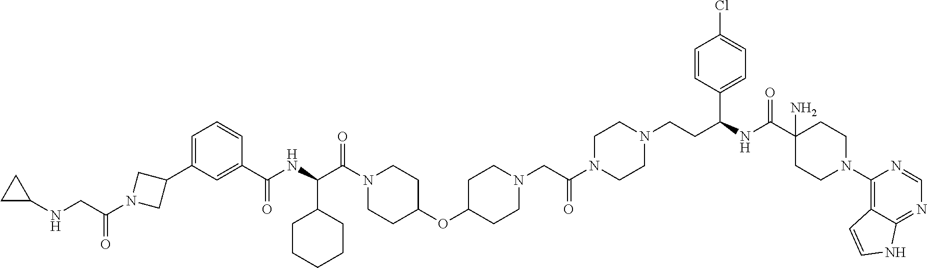 [SMILES: NC1(C(=O)N[C@@H](CCN2CCN(C(=O)CN3CCC(OC4CCN(C(=O)[C@H](NC(=O)c5cccc(C6CN(C(=O)CNC7CC7)C6)c5)C5CCCCC5)CC4)CC3)CC2)c2ccc(Cl)cc2)CCN(c2ncnc3[nH]ccc23)CC1] |
| I-242 | 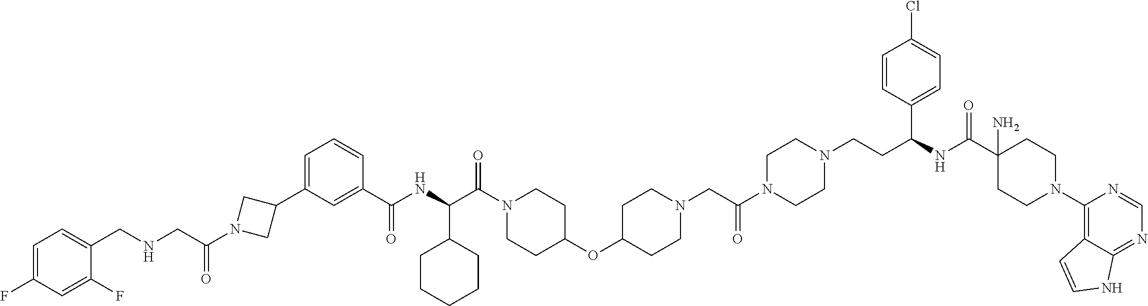 [SMILES: NC1(C(=O)N[C@@H](CCN2CCN(C(=O)CN3CCC(OC4CCN(C(=O)[C@H](NC(=O)c5cccc(C6CN(C(=O)CNCc7ccc(F)cc7F)C6)c5)C5CCCCC5)CC4)CC3)CC2)c2ccc(Cl)cc2)CCN(c2ncnc3[nH]ccc23)CC1] |
| I-243 | 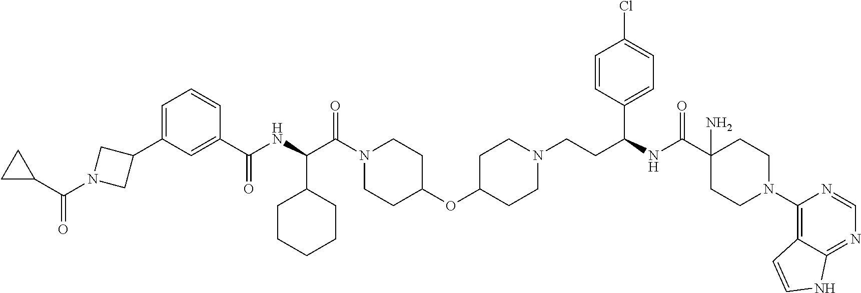 [SMILES: NC1(C(=O)N[C@@H](CCN2CCC(OC3CCN(C(=O)[C@H](NC(=O)c4cccc(C5CN(C(=O)C6CC6)C5)c4)C4CCCCC4)CC3)CC2)c2ccc(Cl)cc2)CCN(c2ncnc3[nH]ccc23)CC1] |
| I-244 | 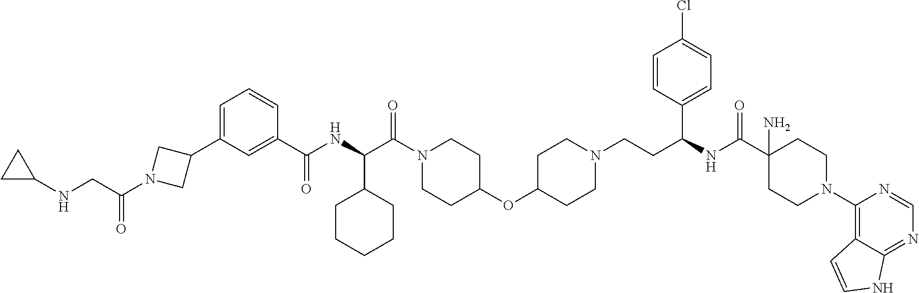 [SMILES: NC1(C(=O)N[C@@H](CCN2CCC(OC3CCN(C(=O)[C@H](NC(=O)c4cccc(C5CN(C(=O)CNC6CC6)C5)c4)C4CCCCC4)CC3)CC2)c2ccc(Cl)cc2)CCN(c2ncnc3[nH]ccc23)CC1] |
| I-245 | 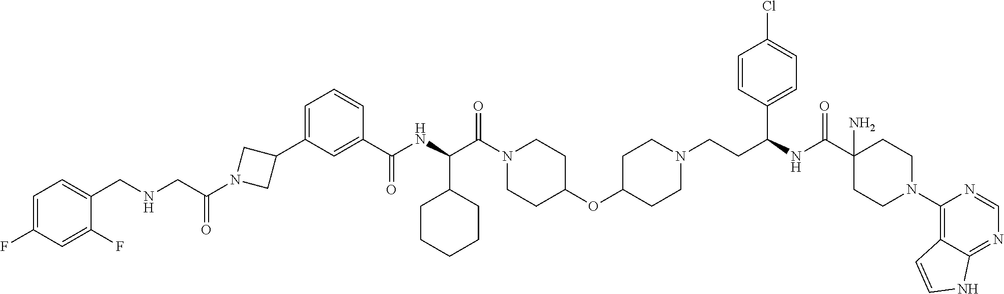 [SMILES: NC1(C(=O)N[C@@H](CCN2CCC(OC3CCN(C(=O)[C@H](NC(=O)c4cccc(C5CN(C(=O)CNCc6ccc(F)cc6F)C5)c4)C4CCCCC4)CC3)CC2)c2ccc(Cl)cc2)CCN(c2ncnc3[nH]ccc23)CC1] |
| I-246 | 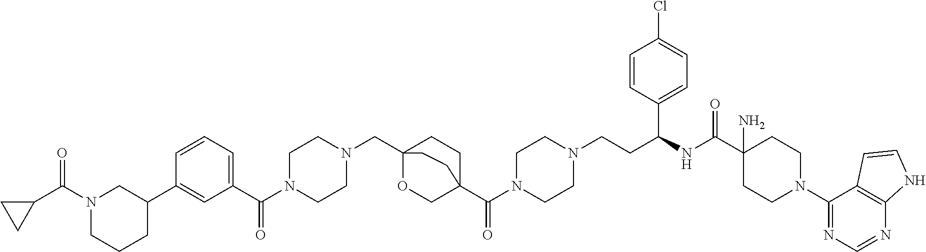 [SMILES: NC1(C(=O)N[C@@H](CCN2CCN(C(=O)C34CCC(CN5CCN(C(=O)c6cccc(C7CCCN(C(=O)C8CC8)C7)c6)CC5)(CC3)OC4)CC2)c2ccc(Cl)cc2)CCN(c2ncnc3[nH]ccc23)CC1] |
| I-247 | 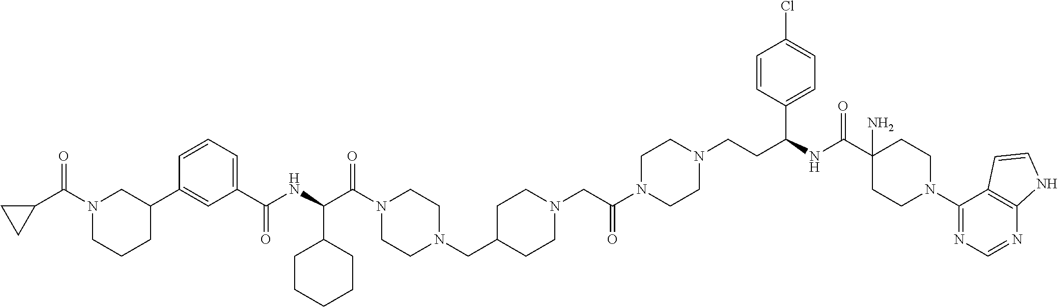 [SMILES: NC1(C(=O)N[C@@H](CCN2CCN(C(=O)CN3CCC(CN4CCN(C(=O)[C@H](NC(=O)c5cccc(C6CCCN(C(=O)C7CC7)C6)c5)C5CCCCC5)CC4)CC3)CC2)c2ccc(Cl)cc2)CCN(c2ncnc3[nH]ccc23)CC1] |
| I-248 | 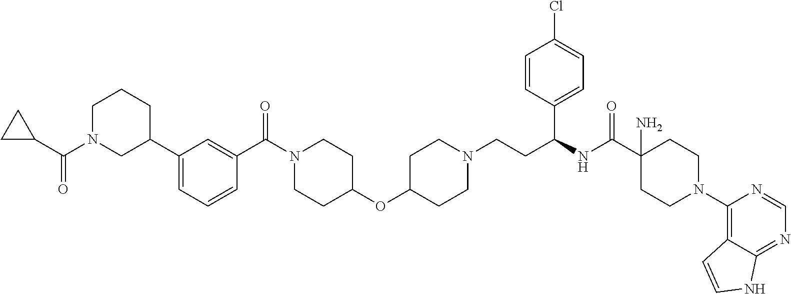 [SMILES: NC1(C(=O)N[C@@H](CCN2CCC(OC3CCN(C(=O)c4cccc(C5CCCN(C(=O)C6CC6)C5)c4)CC3)CC2)c2ccc(Cl)cc2)CCN(c2ncnc3[nH]ccc23)CC1] |
| I-249 | 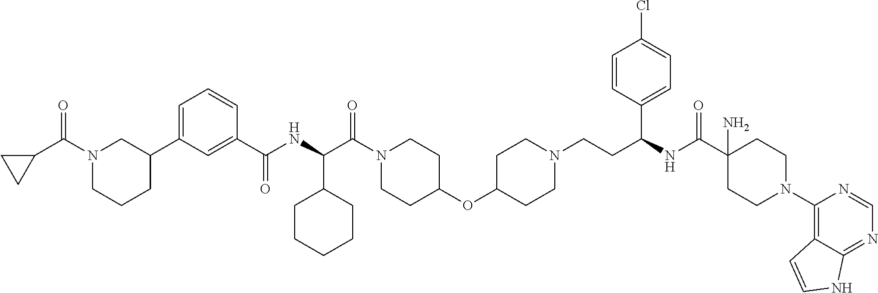 [SMILES: NC1(C(=O)N[C@@H](CCN2CCC(OC3CCN(C(=O)[C@H](NC(=O)c4cccc(C5CCCN(C(=O)C6CC6)C5)c4)C4CCCCC4)CC3)CC2)c2ccc(Cl)cc2)CCN(c2ncnc3[nH]ccc23)CC1] |
| I-250 | 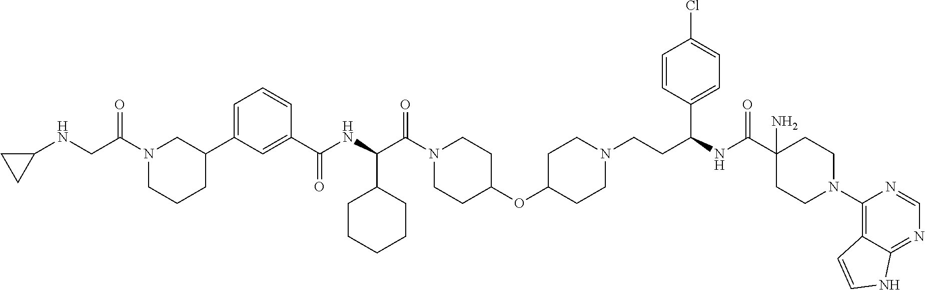 [SMILES: NC1(C(=O)N[C@@H](CCN2CCC(OC3CCN(C(=O)[C@H](NC(=O)c4cccc(C5CCCN(C(=O)CNC6CC6)C5)c4)C4CCCCC4)CC3)CC2)c2ccc(Cl)cc2)CCN(c2ncnc3[nH]ccc23)CC1] |
| I-251 | 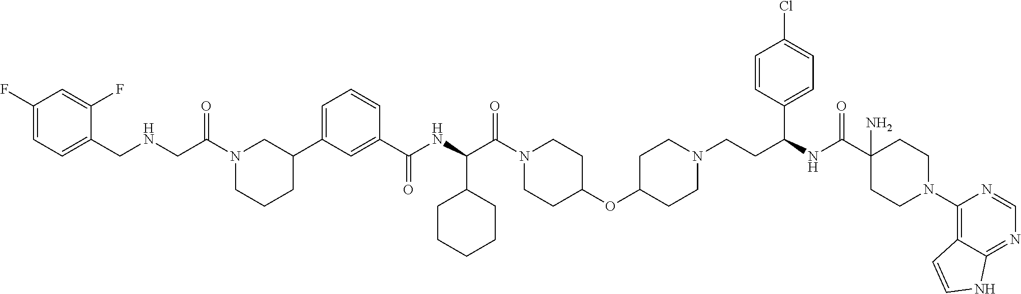 [SMILES: NC1(C(=O)N[C@@H](CCN2CCC(OC3CCN(C(=O)[C@H](NC(=O)c4cccc(C5CCCN(C(=O)CNCc6ccc(F)cc6F)C5)c4)C4CCCCC4)CC3)CC2)c2ccc(Cl)cc2)CCN(c2ncnc3[nH]ccc23)CC1] |
| I-252 | 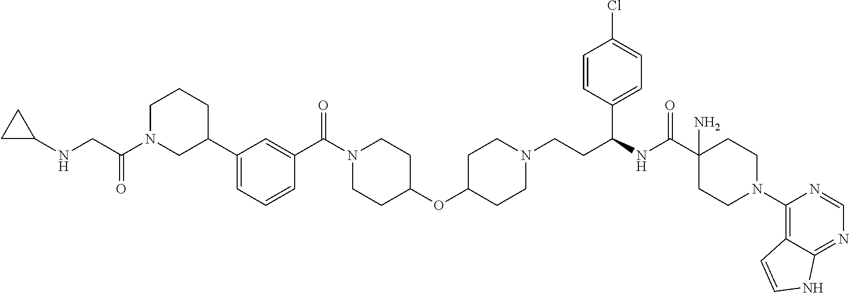 [SMILES: NC1(C(=O)N[C@@H](CCN2CCC(OC3CCN(C(=O)c4cccc(C5CCCN(C(=O)CNC6CC6)C5)c4)CC3)CC2)c2ccc(Cl)cc2)CCN(c2ncnc3[nH]ccc23)CC1] |
| I-253 | 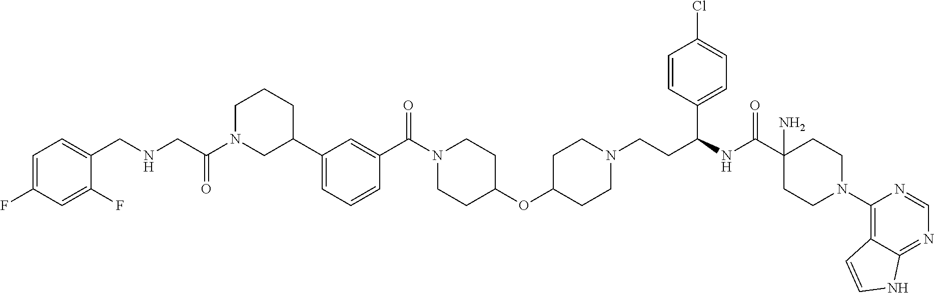 [SMILES: NC1(C(=O)N[C@@H](CCN2CCC(OC3CCN(C(=O)c4cccc(C5CCCN(C(=O)CNCc6ccc(F)cc6F)C5)c4)CC3)CC2)c2ccc(Cl)cc2)CCN(c2ncnc3[nH]ccc23)CC1] |
| I-254 | 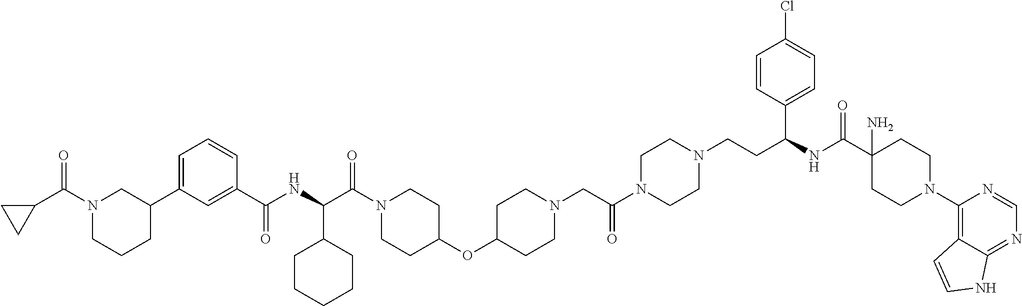 [SMILES: NC1(C(=O)N[C@@H](CCN2CCN(C(=O)CN3CCC(OC4CCN(C(=O)[C@H](NC(=O)c5cccc(C6CCCN(C(=O)C7CC7)C6)c5)C5CCCCC5)CC4)CC3)CC2)c2ccc(Cl)cc2)CCN(c2ncnc3[nH]ccc23)CC1] |
| I-255 | 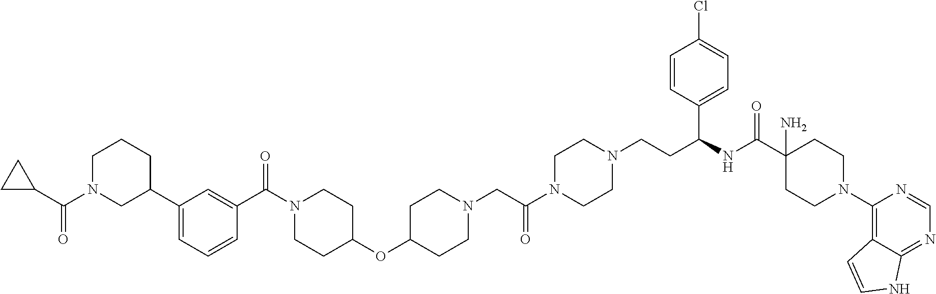 [SMILES: NC1(C(=O)N[C@@H](CCN2CCN(C(=O)CN3CCC(OC4CCN(C(=O)c5cccc(C6CCCN(C(=O)C7CC7)C6)c5)CC4)CC3)CC2)c2ccc(Cl)cc2)CCN(c2ncnc3[nH]ccc23)CC1] |
| I-256 | 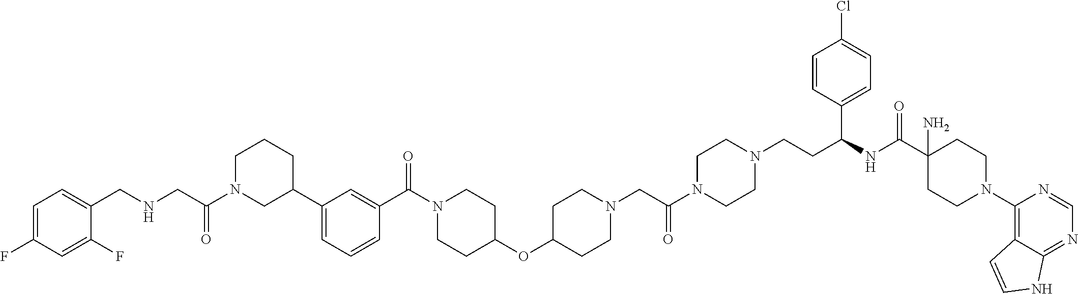 [SMILES: NC1(C(=O)N[C@@H](CCN2CCN(C(=O)CN3CCC(OC4CCN(C(=O)c5cccc(C6CCCN(C(=O)CNCc7ccc(F)cc7F)C6)c5)CC4)CC3)CC2)c2ccc(Cl)cc2)CCN(c2ncnc3[nH]ccc23)CC1] |
| I-257 | 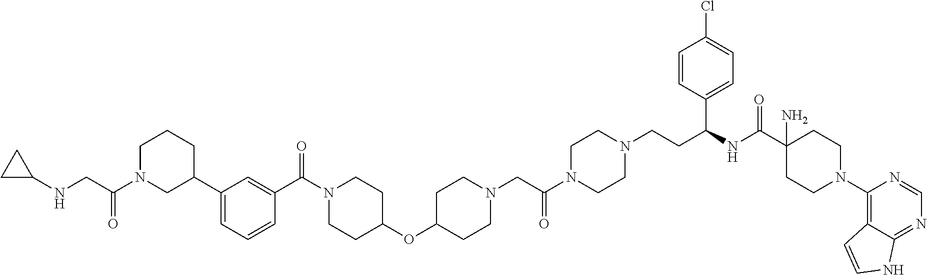 [SMILES: NC1(C(=O)N[C@@H](CCN2CCN(C(=O)CN3CCC(OC4CCN(C(=O)c5cccc(C6CCCN(C(=O)CNC7CC7)C6)c5)CC4)CC3)CC2)c2ccc(Cl)cc2)CCN(c2ncnc3[nH]ccc23)CC1] |
| I-258 | 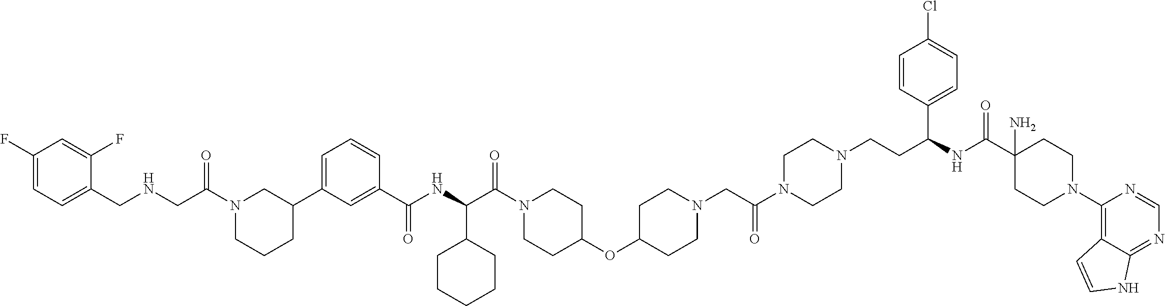 [SMILES: NC1(C(=O)N[C@@H](CCN2CCN(C(=O)CN3CCC(OC4CCN(C(=O)[C@H](NC(=O)c5cccc(C6CCCN(C(=O)CNCc7ccc(F)cc7F)C6)c5)C5CCCCC5)CC4)CC3)CC2)c2ccc(Cl)cc2)CCN(c2ncnc3[nH]ccc23)CC1] |
| I-259 | 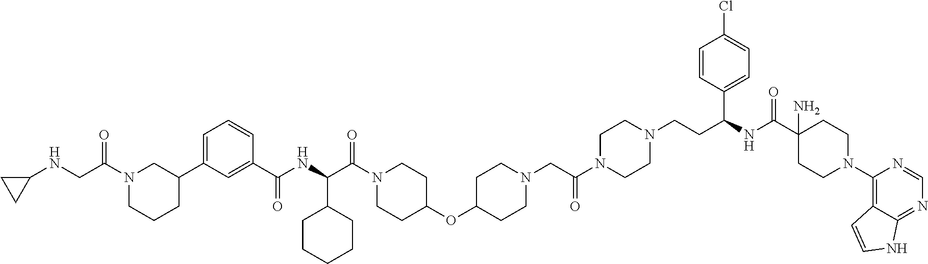 [SMILES: NC1(C(=O)N[C@@H](CCN2CCN(C(=O)CN3CCC(OC4CCN(C(=O)[C@H](NC(=O)c5cccc(C6CCCN(C(=O)CNC7CC7)C6)c5)C5CCCCC5)CC4)CC3)CC2)c2ccc(Cl)cc2)CCN(c2ncnc3[nH]ccc23)CC1] |
| I-260 | 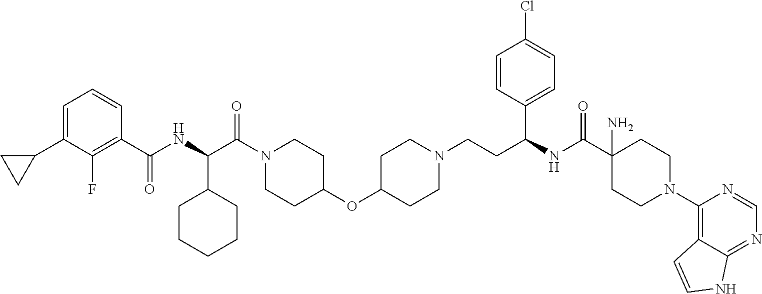 [SMILES: NC1(C(=O)N[C@@H](CCN2CCC(OC3CCN(C(=O)[C@H](NC(=O)c4cccc(C5CC5)c4F)C4CCCCC4)CC3)CC2)c2ccc(Cl)cc2)CCN(c2ncnc3[nH]ccc23)CC1] |
| I-261 | 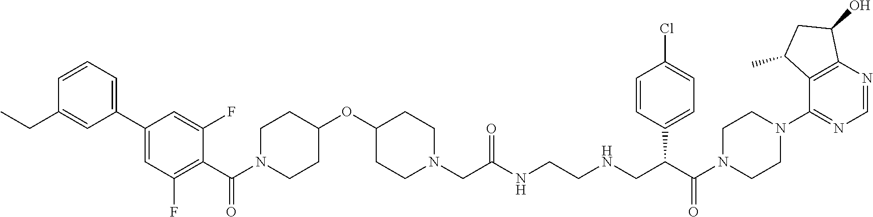 [SMILES: CCc1cccc(-c2cc(F)c(C(=O)N3CCC(OC4CCN(CC(=O)NCCNC[C@@H](C(=O)N5CCN(c6ncnc7c6[C@H](C)C[C@H]7O)CC5)c5ccc(Cl)cc5)CC4)CC3)c(F)c2)c1] |
| I-262 | 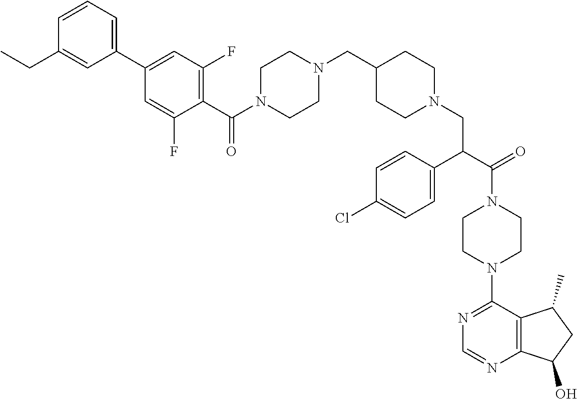 [SMILES: CCc1cccc(-c2cc(F)c(C(=O)N3CCN(CC4CCN(CC(C(=O)N5CCN(c6ncnc7c6[C@H](C)C[C@H]7O)CC5)c5ccc(Cl)cc5)CC4)CC3)c(F)c2)c1] |
| I-263 | 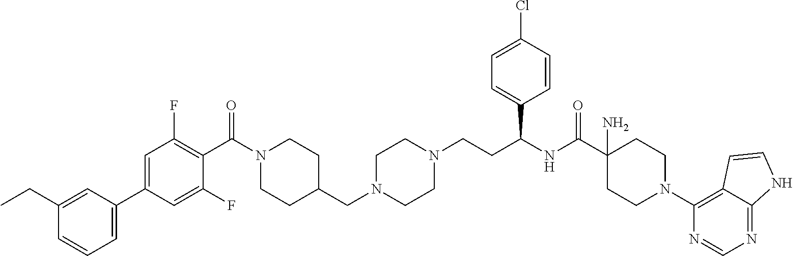 [SMILES: CCc1cccc(-c2cc(F)c(C(=O)N3CCC(CN4CCN(CC[C@H](NC(=O)C5(N)CCN(c6ncnc7[nH]ccc67)CC5)c5ccc(Cl)cc5)CC4)CC3)c(F)c2)c1] |
| I-264 | 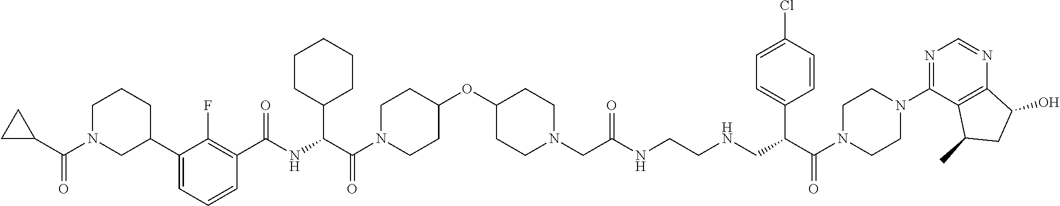 [SMILES: C[C@@H]1C[C@@H](O)c2ncnc(N3CCN(C(=O)[C@H](CNCCNC(=O)CN4CCC(OC5CCN(C(=O)[C@H](NC(=O)c6cccc(C7CCCN(C(=O)C8CC8)C7)c6F)C6CCCCC6)CC5)CC4)c4ccc(Cl)cc4)CC3)c21] |
| I-265 | 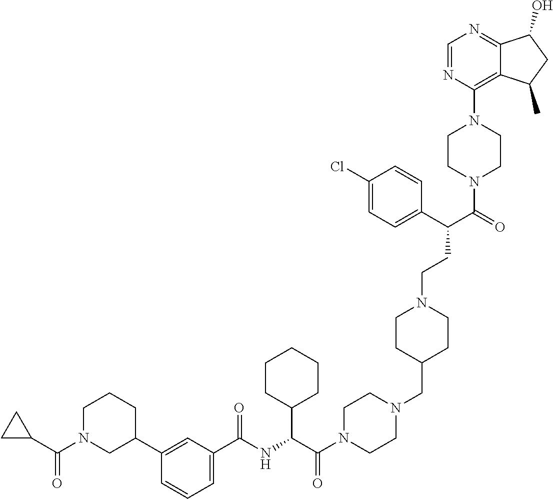 [SMILES: C[C@@H]1C[C@@H](O)c2ncnc(N3CCN(C(=O)[C@H](CCN4CCC(CN5CCN(C(=O)[C@H](NC(=O)c6cccc(C7CCCN(C(=O)C8CC8)C7)c6)C6CCCCC6)CC5)CC4)c4ccc(Cl)cc4)CC3)c21] |
| I-266 | 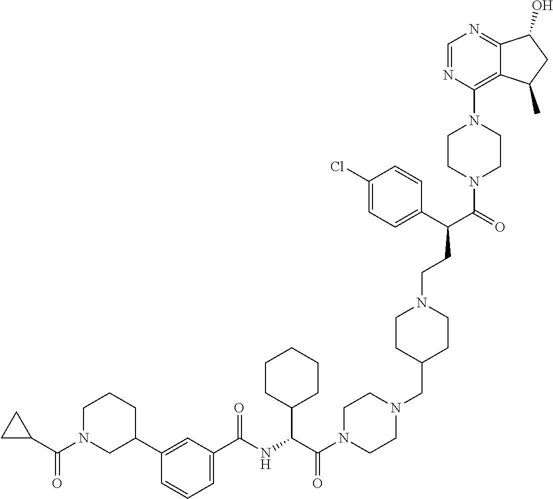 [SMILES: C[C@@H]1C[C@@H](O)c2ncnc(N3CCN(C(=O)[C@@H](CCN4CCC(CN5CCN(C(=O)[C@H](NC(=O)c6cccc(C7CCCN(C(=O)C8CC8)C7)c6)C6CCCCC6)CC5)CC4)c4ccc(Cl)cc4)CC3)c21] |
| I-268 | 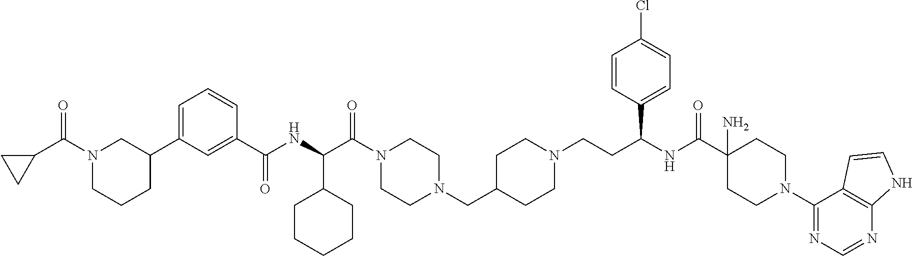 [SMILES: NC1(C(=O)N[C@@H](CCN2CCC(CN3CCN(C(=O)[C@H](NC(=O)c4cccc(C5CCCN(C(=O)C6CC6)C5)c4)C4CCCCC4)CC3)CC2)c2ccc(Cl)cc2)CCN(c2ncnc3[nH]ccc23)CC1] |
| I-269 | 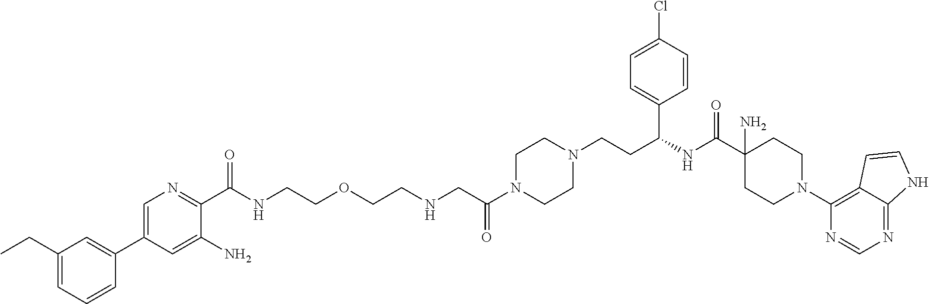 [SMILES: CCc1cccc(-c2cnc(C(=O)NCCOCCNCC(=O)N3CCN(CC[C@@H](NC(=O)C4(N)CCN(c5ncnc6[nH]ccc56)CC4)c4ccc(Cl)cc4)CC3)c(N)c2)c1] |
| I-270 | 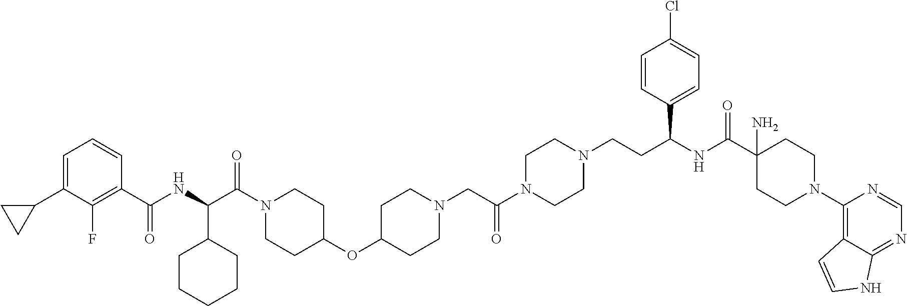 [SMILES: NC1(C(=O)N[C@@H](CCN2CCN(C(=O)CN3CCC(OC4CCN(C(=O)[C@H](NC(=O)c5cccc(C6CC6)c5F)C5CCCCC5)CC4)CC3)CC2)c2ccc(Cl)cc2)CCN(c2ncnc3[nH]ccc23)CC1] |
| I-271 | 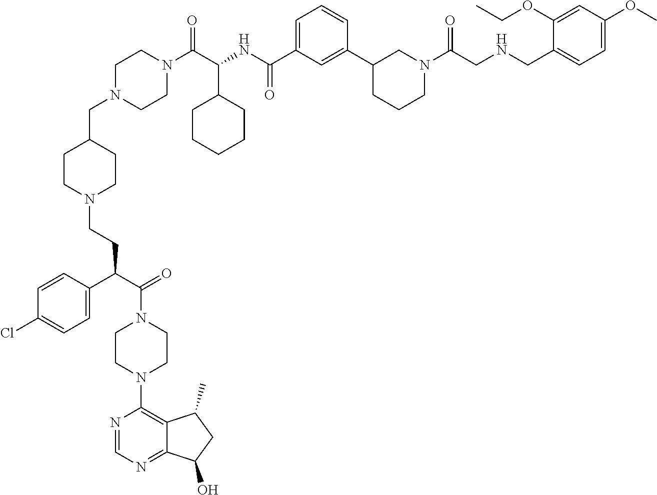 [SMILES: CCOc1cc(OC)ccc1CNCC(=O)N1CCCC(c2cccc(C(=O)N[C@@H](C(=O)N3CCN(CC4CCN(CC[C@@H](C(=O)N5CCN(c6ncnc7c6[C@H](C)C[C@H]7O)CC5)c5ccc(Cl)cc5)CC4)CC3)C3CCCCC3)c2)C1] |
| I-272 | 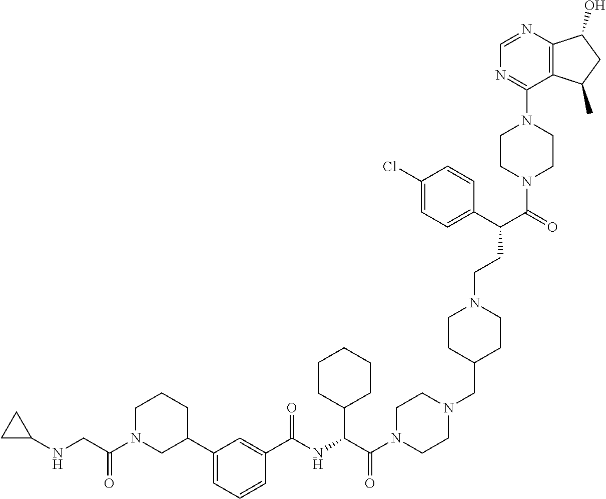 [SMILES: C[C@@H]1C[C@@H](O)c2ncnc(N3CCN(C(=O)[C@H](CCN4CCC(CN5CCN(C(=O)[C@H](NC(=O)c6cccc(C7CCCN(C(=O)CNC8CC8)C7)c6)C6CCCCC6)CC5)CC4)c4ccc(Cl)cc4)CC3)c21] |
| I-273 | 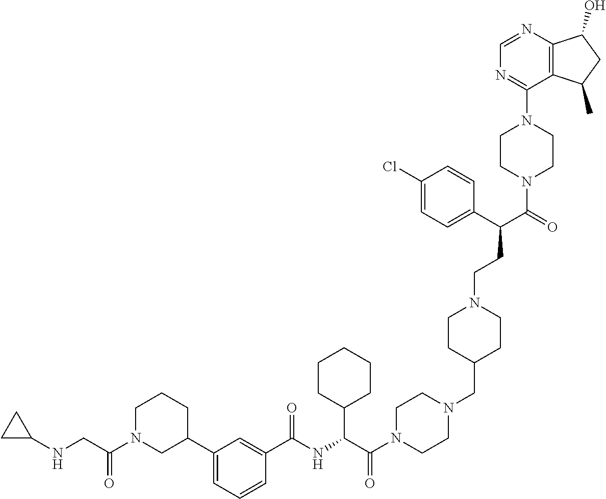 [SMILES: C[C@@H]1C[C@@H](O)c2ncnc(N3CCN(C(=O)[C@@H](CCN4CCC(CN5CCN(C(=O)[C@H](NC(=O)c6cccc(C7CCCN(C(=O)CNC8CC8)C7)c6)C6CCCCC6)CC5)CC4)c4ccc(Cl)cc4)CC3)c21] |
| I-274 | 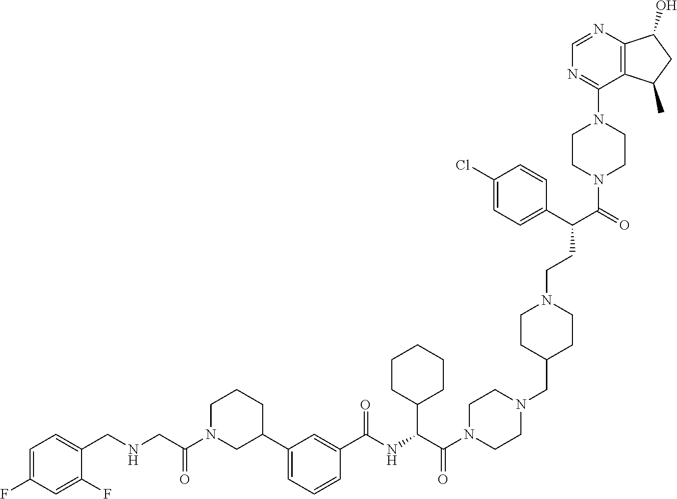 [SMILES: C[C@@H]1C[C@@H](O)c2ncnc(N3CCN(C(=O)[C@H](CCN4CCC(CN5CCN(C(=O)[C@H](NC(=O)c6cccc(C7CCCN(C(=O)CNCc8ccc(F)cc8F)C7)c6)C6CCCCC6)CC5)CC4)c4ccc(Cl)cc4)CC3)c21] |
| I-275 | 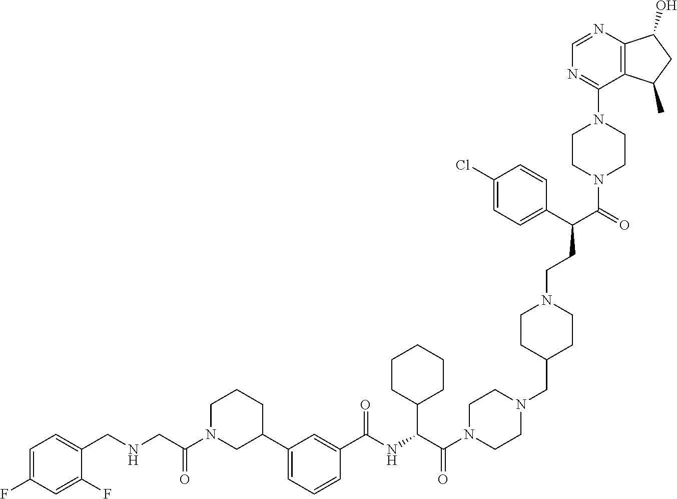 [SMILES: C[C@@H]1C[C@@H](O)c2ncnc(N3CCN(C(=O)[C@@H](CCN4CCC(CN5CCN(C(=O)[C@H](NC(=O)c6cccc(C7CCCN(C(=O)CNCc8ccc(F)cc8F)C7)c6)C6CCCCC6)CC5)CC4)c4ccc(Cl)cc4)CC3)c21] |
| I-276 | 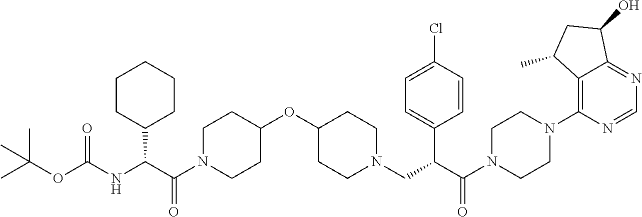 [SMILES: C[C@@H]1C[C@@H](O)c2ncnc(N3CCN(C(=O)[C@H](CN4CCC(OC5CCN(C(=O)[C@H](NC(=O)OC(C)(C)C)C6CCCCC6)CC5)CC4)c4ccc(Cl)cc4)CC3)c21] |
| I-277 | 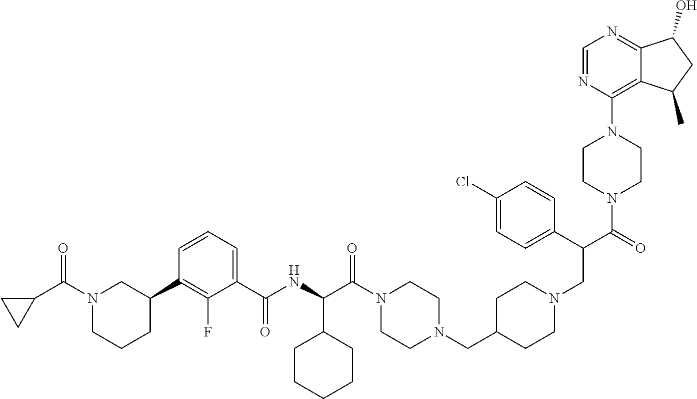 [SMILES: C[C@@H]1C[C@@H](O)c2ncnc(N3CCN(C(=O)C(CN4CCC(CN5CCN(C(=O)[C@H](NC(=O)c6cccc([C@H]7CCCN(C(=O)C8CC8)C7)c6F)C6CCCCC6)CC5)CC4)c4ccc(Cl)cc4)CC3)c21] |
| I-278 | 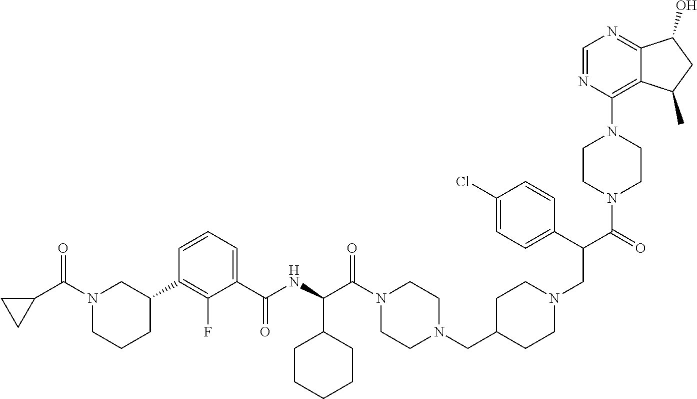 [SMILES: C[C@@H]1C[C@@H](O)c2ncnc(N3CCN(C(=O)C(CN4CCC(CN5CCN(C(=O)[C@H](NC(=O)c6cccc([C@@H]7CCCN(C(=O)C8CC8)C7)c6F)C6CCCCC6)CC5)CC4)c4ccc(Cl)cc4)CC3)c21] |
| I-279 | 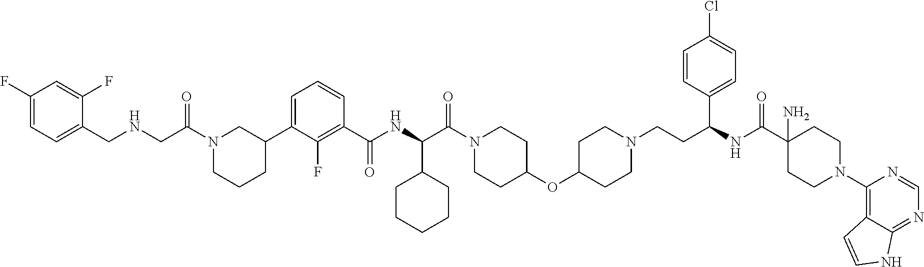 [SMILES: NC1(C(=O)N[C@@H](CCN2CCC(OC3CCN(C(=O)[C@H](NC(=O)c4cccc(C5CCCN(C(=O)CNCc6ccc(F)cc6F)C5)c4F)C4CCCCC4)CC3)CC2)c2ccc(Cl)cc2)CCN(c2ncnc3[nH]ccc23)CC1] |
| I-280 | 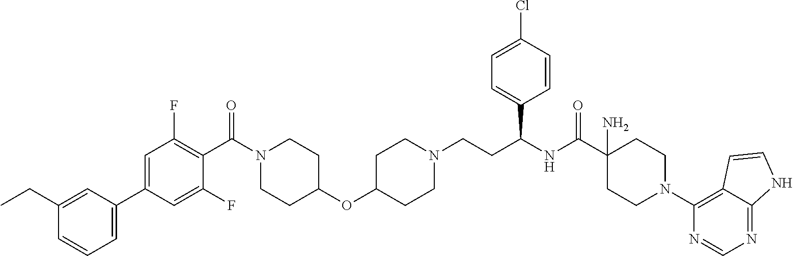 [SMILES: CCc1cccc(-c2cc(F)c(C(=O)N3CCC(OC4CCN(CC[C@H](NC(=O)C5(N)CCN(c6ncnc7[nH]ccc67)CC5)c5ccc(Cl)cc5)CC4)CC3)c(F)c2)c1] |
| I-281 | 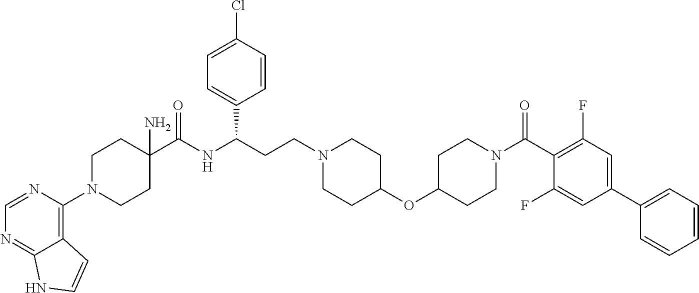 [SMILES: NC1(C(=O)N[C@@H](CCN2CCC(OC3CCN(C(=O)c4c(F)cc(-c5ccccc5)cc4F)CC3)CC2)c2ccc(Cl)cc2)CCN(c2ncnc3[nH]ccc23)CC1] |
| I-282 | 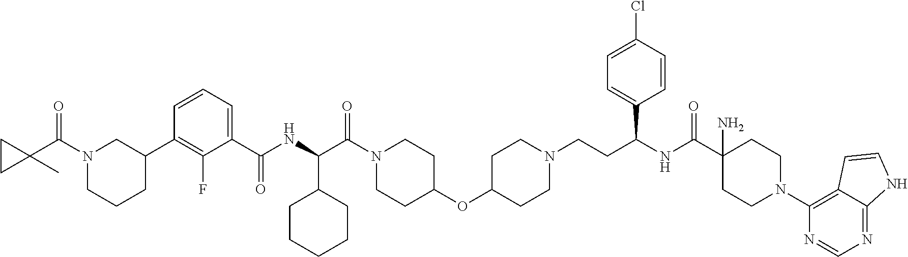 [SMILES: CC1(C(=O)N2CCCC(c3cccc(C(=O)N[C@@H](C(=O)N4CCC(OC5CCN(CC[C@H](NC(=O)C6(N)CCN(c7ncnc8[nH]ccc78)CC6)c6ccc(Cl)cc6)CC5)CC4)C4CCCCC4)c3F)C2)CC1] |
| I-283 | 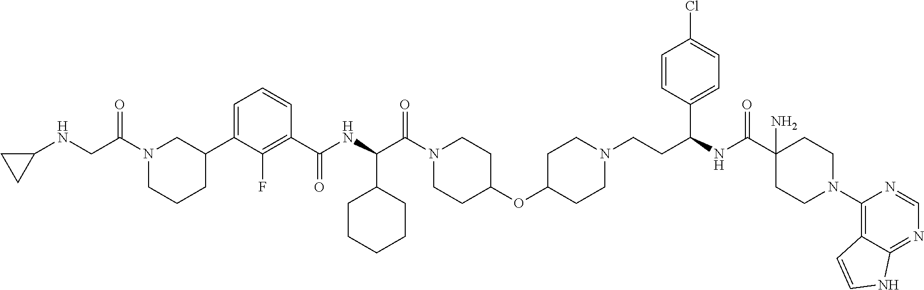 [SMILES: NC1(C(=O)N[C@@H](CCN2CCC(OC3CCN(C(=O)[C@H](NC(=O)c4cccc(C5CCCN(C(=O)CNC6CC6)C5)c4F)C4CCCCC4)CC3)CC2)c2ccc(Cl)cc2)CCN(c2ncnc3[nH]ccc23)CC1] |
| I-284 | 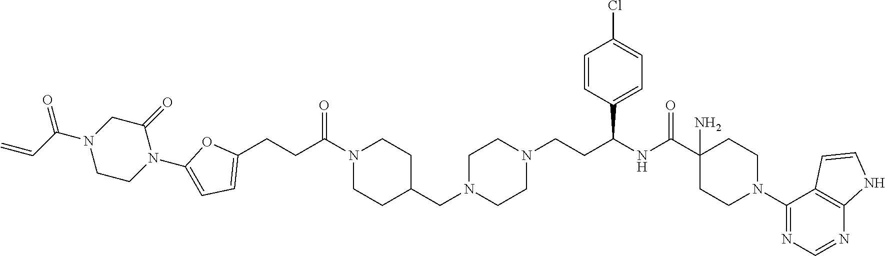 [SMILES: C=CC(=O)N1CCN(c2ccc(CCC(=O)N3CCC(CN4CCN(CC[C@H](NC(=O)C5(N)CCN(c6ncnc7[nH]ccc67)CC5)c5ccc(Cl)cc5)CC4)CC3)o2)C(=O)C1] |
| I-285 | 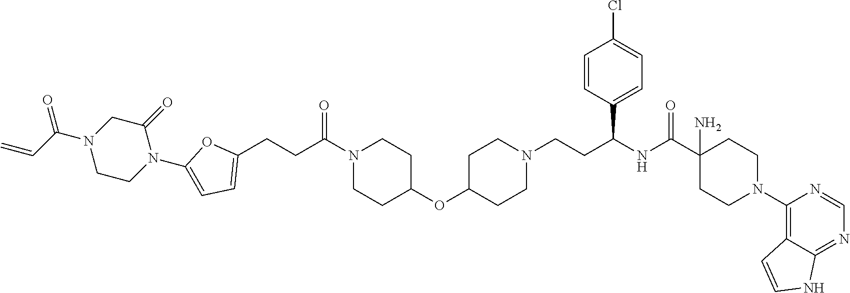 [SMILES: C=CC(=O)N1CCN(c2ccc(CCC(=O)N3CCC(OC4CCN(CC[C@H](NC(=O)C5(N)CCN(c6ncnc7[nH]ccc67)CC5)c5ccc(Cl)cc5)CC4)CC3)o2)C(=O)C1] |
| I-286 | 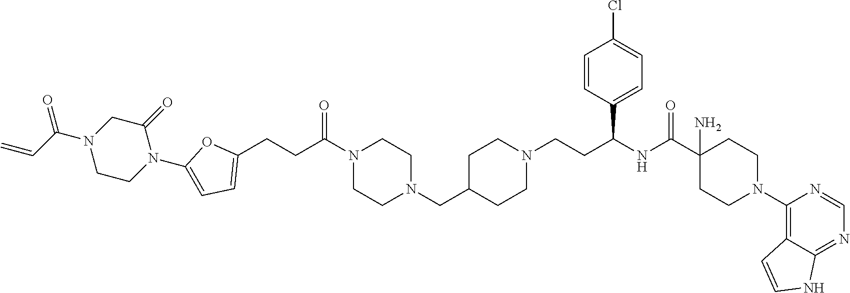 [SMILES: C=CC(=O)N1CCN(c2ccc(CCC(=O)N3CCN(CC4CCN(CC[C@H](NC(=O)C5(N)CCN(c6ncnc7[nH]ccc67)CC5)c5ccc(Cl)cc5)CC4)CC3)o2)C(=O)C1] |
| I-287 | 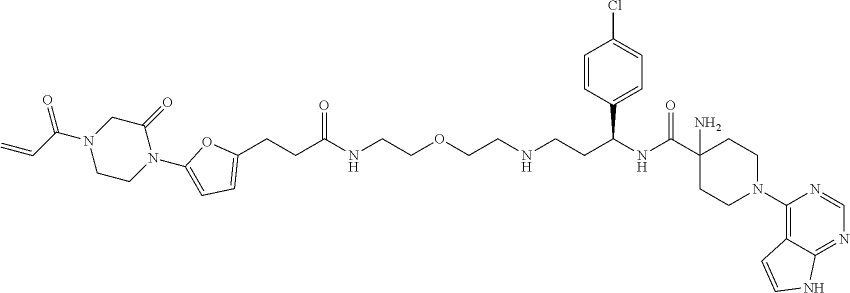 [SMILES: C=CC(=O)N1CCN(c2ccc(CCC(=O)NCCOCCNCC[C@H](NC(=O)C3(N)CCN(c4ncnc5[nH]ccc45)CC3)c3ccc(Cl)cc3)o2)C(=O)C1] |
| I-288 | 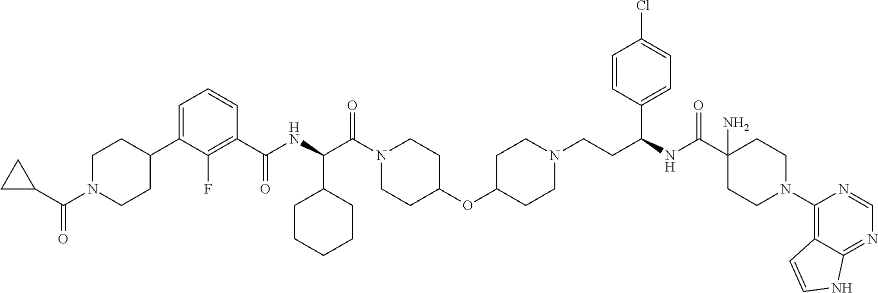 [SMILES: NC1(C(=O)N[C@@H](CCN2CCC(OC3CCN(C(=O)[C@H](NC(=O)c4cccc(C5CCN(C(=O)C6CC6)CC5)c4F)C4CCCCC4)CC3)CC2)c2ccc(Cl)cc2)CCN(c2ncnc3[nH]ccc23)CC1] |
| I-289 | 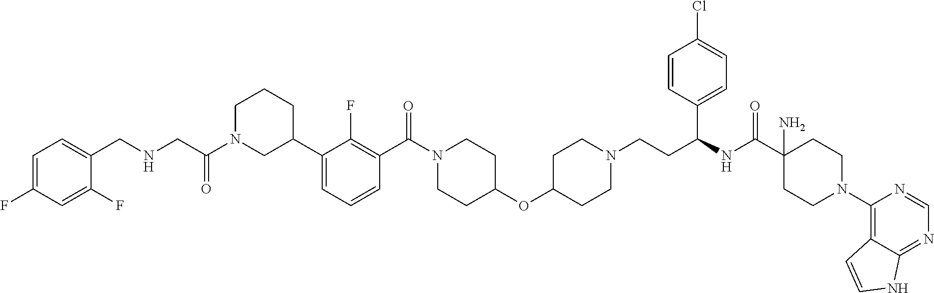 [SMILES: NC1(C(=O)N[C@@H](CCN2CCC(OC3CCN(C(=O)c4cccc(C5CCCN(C(=O)CNCc6ccc(F)cc6F)C5)c4F)CC3)CC2)c2ccc(Cl)cc2)CCN(c2ncnc3[nH]ccc23)CC1] |
| I-290 | 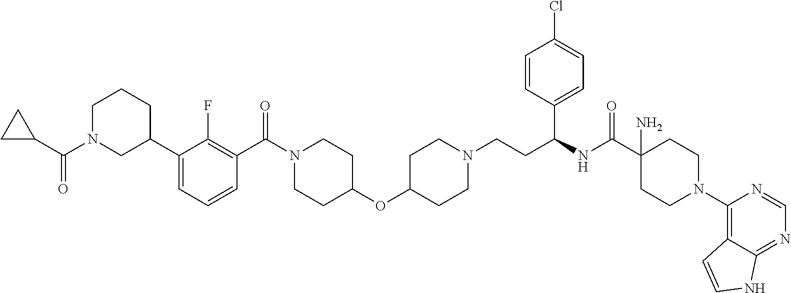 [SMILES: NC1(C(=O)N[C@@H](CCN2CCC(OC3CCN(C(=O)c4cccc(C5CCCN(C(=O)C6CC6)C5)c4F)CC3)CC2)c2ccc(Cl)cc2)CCN(c2ncnc3[nH]ccc23)CC1] |
| I-291 | 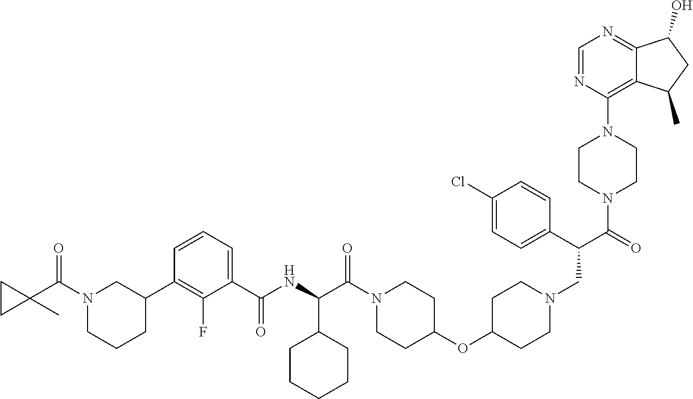 [SMILES: C[C@@H]1C[C@@H](O)c2ncnc(N3CCN(C(=O)[C@H](CN4CCC(OC5CCN(C(=O)[C@H](NC(=O)c6cccc(C7CCCN(C(=O)C8(C)CC8)C7)c6F)C6CCCCC6)CC5)CC4)c4ccc(Cl)cc4)CC3)c21] |
| I-292 | 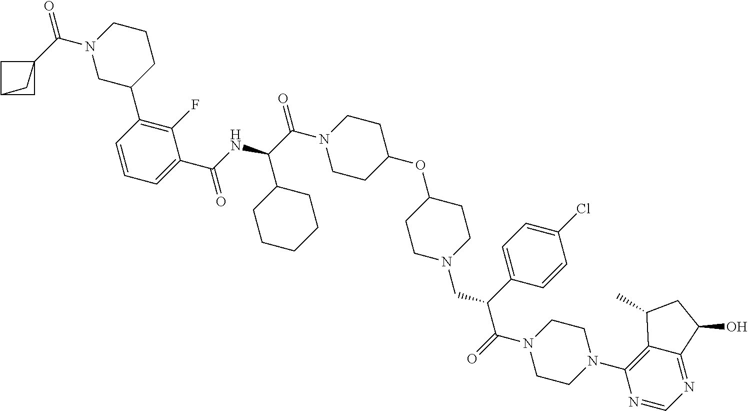 [SMILES: C[C@@H]1C[C@@H](O)c2ncnc(N3CCN(C(=O)[C@H](CN4CCC(OC5CCN(C(=O)[C@H](NC(=O)c6cccc(C7CCCN(C(=O)C89CC(C8)C9)C7)c6F)C6CCCCC6)CC5)CC4)c4ccc(Cl)cc4)CC3)c21] |
| I-293 | 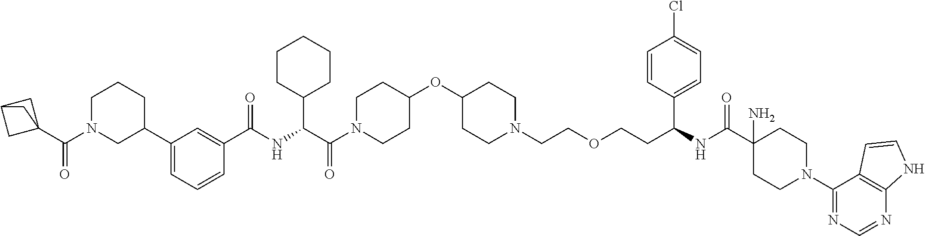 [SMILES: NC1(C(=O)N[C@@H](CCOCCN2CCC(OC3CCN(C(=O)[C@H](NC(=O)c4cccc(C5CCCN(C(=O)C67CC(C6)C7)C5)c4)C4CCCCC4)CC3)CC2)c2ccc(Cl)cc2)CCN(c2ncnc3[nH]ccc23)CC1] |
| I-294 | 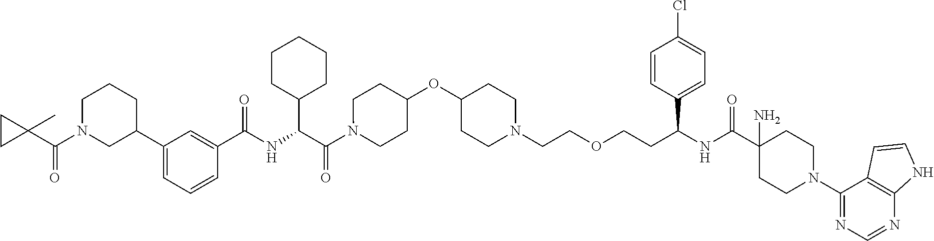 [SMILES: CC1(C(=O)N2CCCC(c3cccc(C(=O)N[C@@H](C(=O)N4CCC(OC5CCN(CCOCC[C@H](NC(=O)C6(N)CCN(c7ncnc8[nH]ccc78)CC6)c6ccc(Cl)cc6)CC5)CC4)C4CCCCC4)c3)C2)CC1] |
| I-295 | 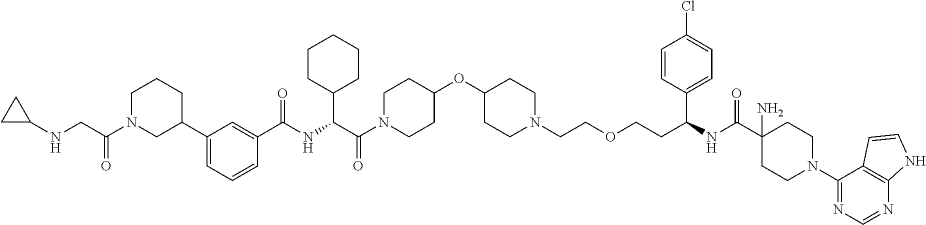 [SMILES: NC1(C(=O)N[C@@H](CCOCCN2CCC(OC3CCN(C(=O)[C@H](NC(=O)c4cccc(C5CCCN(C(=O)CNC6CC6)C5)c4)C4CCCCC4)CC3)CC2)c2ccc(Cl)cc2)CCN(c2ncnc3[nH]ccc23)CC1] |
| I-296 | 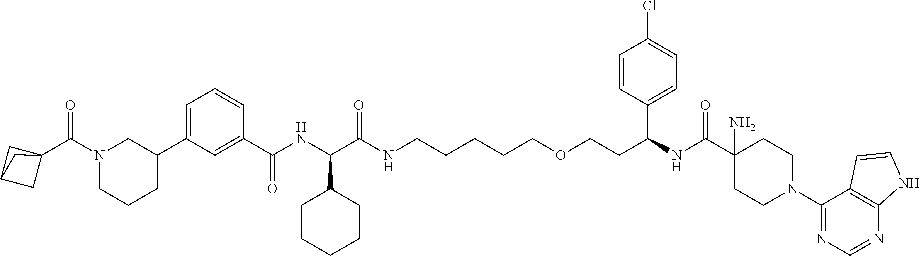 [SMILES: NC1(C(=O)N[C@@H](CCOCCCCCNC(=O)[C@H](NC(=O)c2cccc(C3CCCN(C(=O)C45CC(C4)C5)C3)c2)C2CCCCC2)c2ccc(Cl)cc2)CCN(c2ncnc3[nH]ccc23)CC1] |
| I-297 | 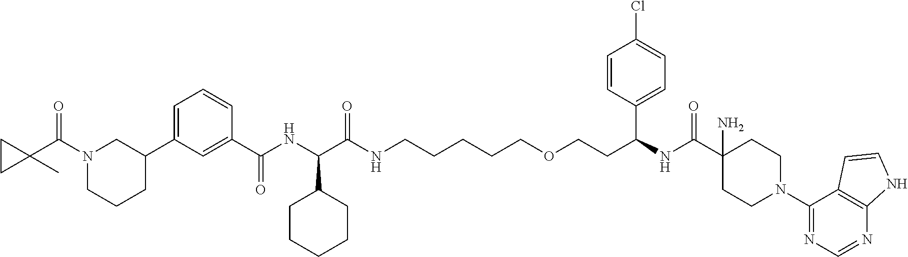 [SMILES: CC1(C(=O)N2CCCC(c3cccc(C(=O)N[C@@H](C(=O)NCCCCCOCC[C@H](NC(=O)C4(N)CCN(c5ncnc6[nH]ccc56)CC4)c4ccc(Cl)cc4)C4CCCCC4)c3)C2)CC1] |
| I-298 | 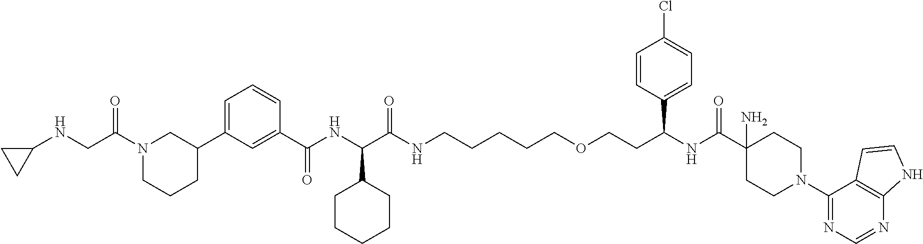 [SMILES: NC1(C(=O)N[C@@H](CCOCCCCCNC(=O)[C@H](NC(=O)c2cccc(C3CCCN(C(=O)CNC4CC4)C3)c2)C2CCCCC2)c2ccc(Cl)cc2)CCN(c2ncnc3[nH]ccc23)CC1] |
| I-299 | 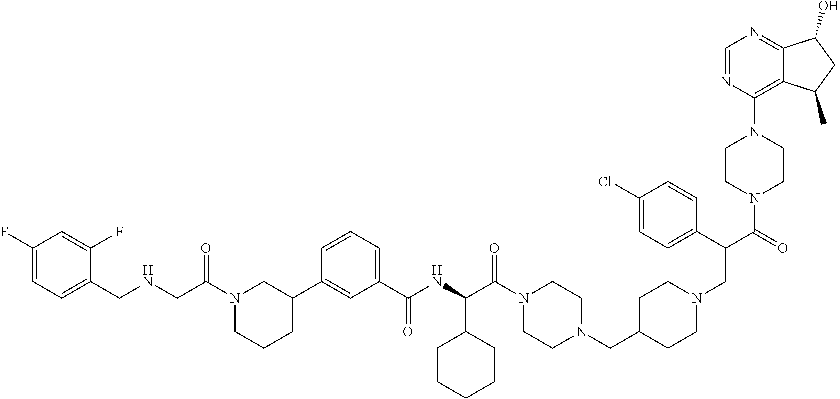 [SMILES: C[C@@H]1C[C@@H](O)c2ncnc(N3CCN(C(=O)C(CN4CCC(CN5CCN(C(=O)[C@H](NC(=O)c6cccc(C7CCCN(C(=O)CNCc8ccc(F)cc8F)C7)c6)C6CCCCC6)CC5)CC4)c4ccc(Cl)cc4)CC3)c21] |
| I-300 | 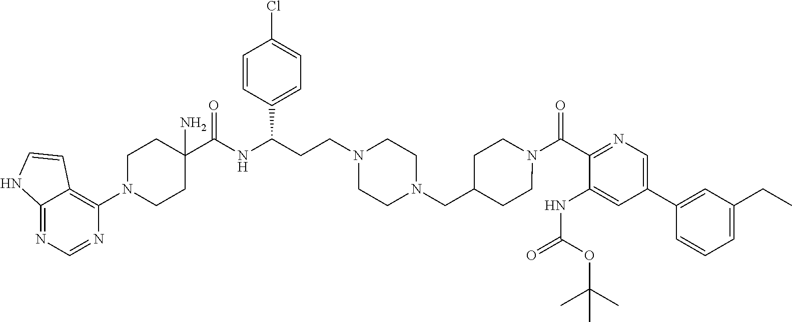 [SMILES: CCc1cccc(-c2cnc(C(=O)N3CCC(CN4CCN(CC[C@H](NC(=O)C5(N)CCN(c6ncnc7[nH]ccc67)CC5)c5ccc(Cl)cc5)CC4)CC3)c(NC(=O)OC(C)(C)C)c2)c1] |
| I-301 | 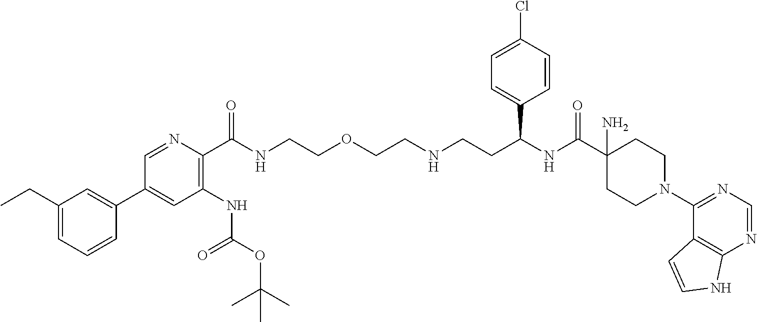 [SMILES: CCc1cccc(-c2cnc(C(=O)NCCOCCNCC[C@H](NC(=O)C3(N)CCN(c4ncnc5[nH]ccc45)CC3)c3ccc(Cl)cc3)c(NC(=O)OC(C)(C)C)c2)c1] |
| I-302 | 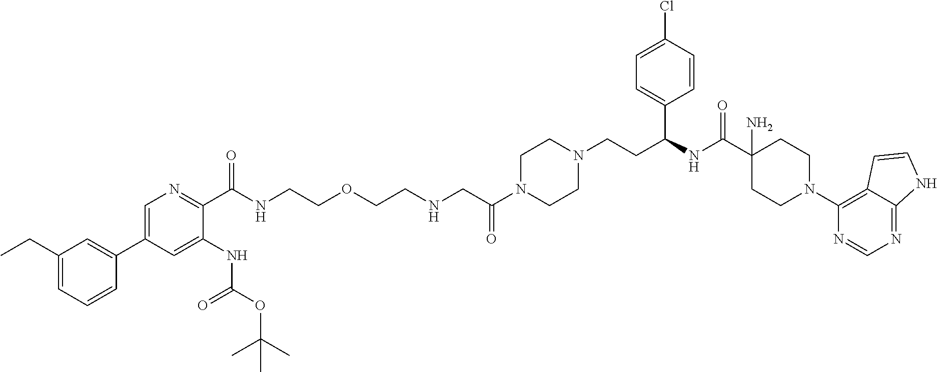 [SMILES: CCc1cccc(-c2cnc(C(=O)NCCOCCNCC(=O)N3CCN(CC[C@H](NC(=O)C4(N)CCN(c5ncnc6[nH]ccc56)CC4)c4ccc(Cl)cc4)CC3)c(NC(=O)OC(C)(C)C)c2)c1] |
| I-303 | 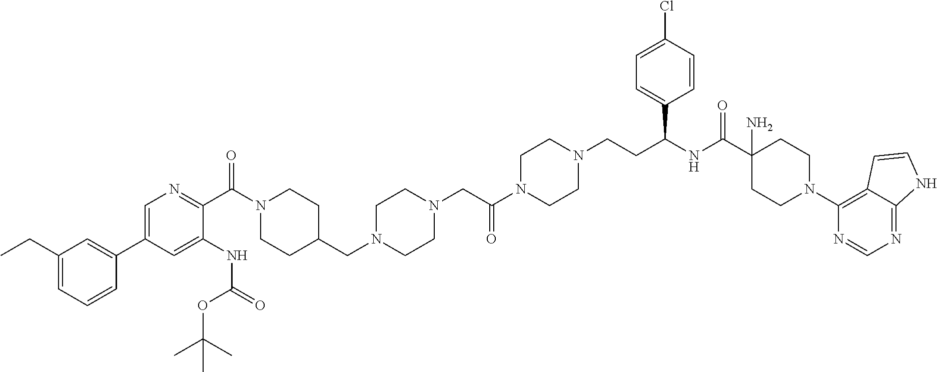 [SMILES: CCc1cccc(-c2cnc(C(=O)N3CCC(CN4CCN(CC(=O)N5CCN(CC[C@H](NC(=O)C6(N)CCN(c7ncnc8[nH]ccc78)CC6)c6ccc(Cl)cc6)CC5)CC4)CC3)c(NC(=O)OC(C)(C)C)c2)c1] |
| I-304 | 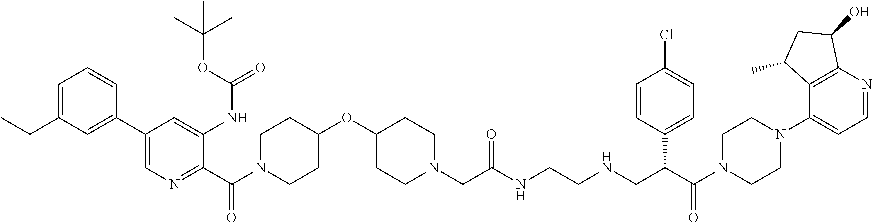 [SMILES: CCc1cccc(-c2cnc(C(=O)N3CCC(OC4CCN(CC(=O)NCCNC[C@@H](C(=O)N5CCN(c6ccnc7c6[C@H](C)C[C@H]7O)CC5)c5ccc(Cl)cc5)CC4)CC3)c(NC(=O)OC(C)(C)C)c2)c1] |
| I-305 | 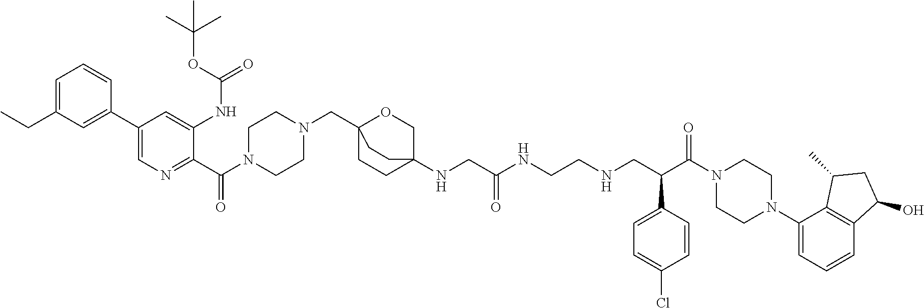 [SMILES: CCc1cccc(-c2cnc(C(=O)N3CCN(CC45CCC(NCC(=O)NCCNC[C@@H](C(=O)N6CCN(c7cccc8c7[C@H](C)C[C@H]8O)CC6)c6ccc(Cl)cc6)(CC4)CO5)CC3)c(NC(=O)OC(C)(C)C)c2)c1] |
| I-306 | 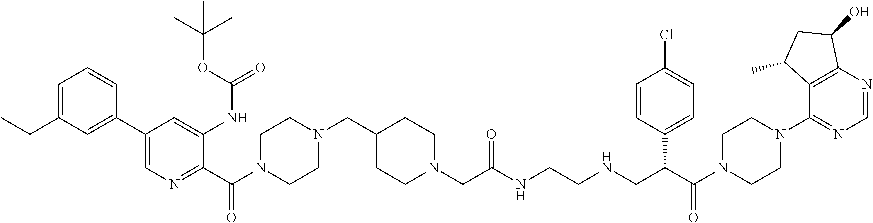 [SMILES: CCc1cccc(-c2cnc(C(=O)N3CCN(CC4CCN(CC(=O)NCCNC[C@@H](C(=O)N5CCN(c6ncnc7c6[C@H](C)C[C@H]7O)CC5)c5ccc(Cl)cc5)CC4)CC3)c(NC(=O)OC(C)(C)C)c2)c1] |
| I-307 | 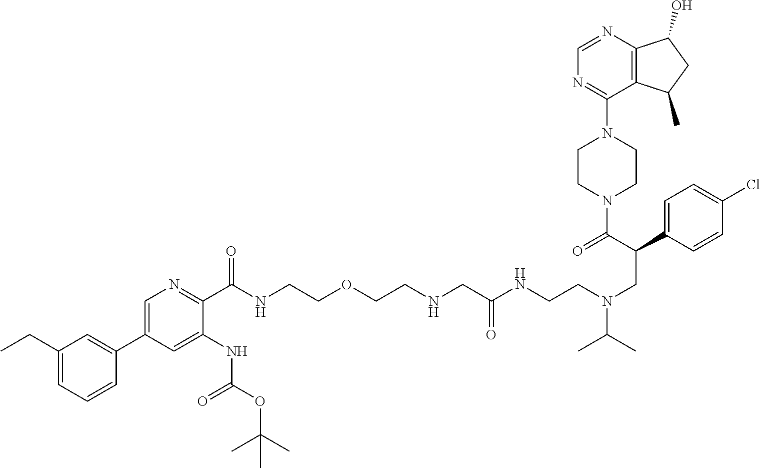 [SMILES: CCc1cccc(-c2cnc(C(=O)NCCOCCNCC(=O)NCCN(C[C@@H](C(=O)N3CCN(c4ncnc5c4[C@H](C)C[C@H]5O)CC3)c3ccc(Cl)cc3)C(C)C)c(NC(=O)OC(C)(C)C)c2)c1] |
| I-308 | 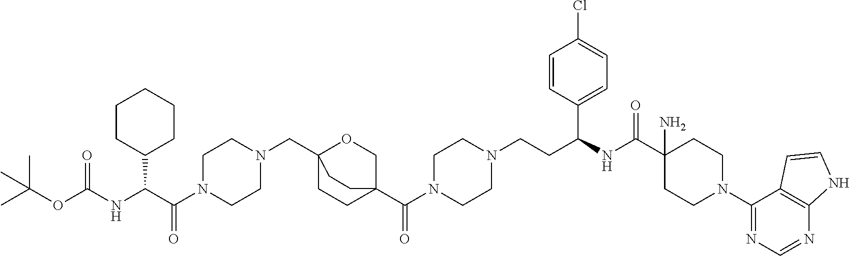 [SMILES: CC(C)(C)OC(=O)N[C@@H](C(=O)N1CCN(CC23CCC(C(=O)N4CCN(CC[C@H](NC(=O)C5(N)CCN(c6ncnc7[nH]ccc67)CC5)c5ccc(Cl)cc5)CC4)(CC2)CO3)CC1)C1CCCCC1] |
| I-309 | 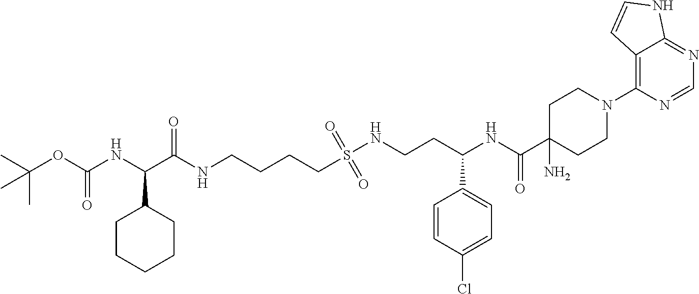 [SMILES: CC(C)(C)OC(=O)N[C@@H](C(=O)NCCCCS(=O)(=O)NCC[C@H](NC(=O)C1(N)CCN(c2ncnc3[nH]ccc23)CC1)c1ccc(Cl)cc1)C1CCCCC1] |
| I-310 | 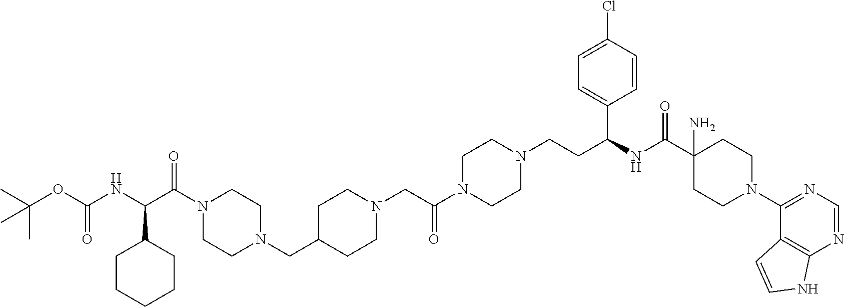 [SMILES: CC(C)(C)OC(=O)N[C@@H](C(=O)N1CCN(CC2CCN(CC(=O)N3CCN(CC[C@H](NC(=O)C4(N)CCN(c5ncnc6[nH]ccc56)CC4)c4ccc(Cl)cc4)CC3)CC2)CC1)C1CCCCC1] |
| I-311 | 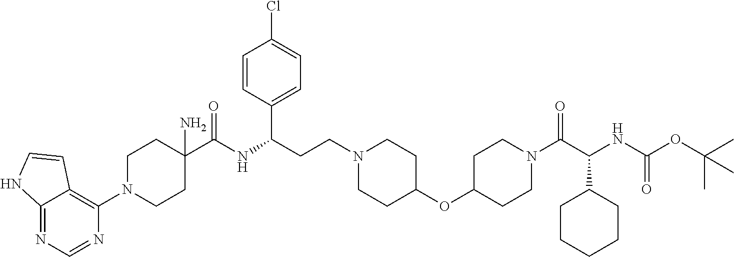 [SMILES: CC(C)(C)OC(=O)N[C@@H](C(=O)N1CCC(OC2CCN(CC[C@H](NC(=O)C3(N)CCN(c4ncnc5[nH]ccc45)CC3)c3ccc(Cl)cc3)CC2)CC1)C1CCCCC1] |
| I-312 | 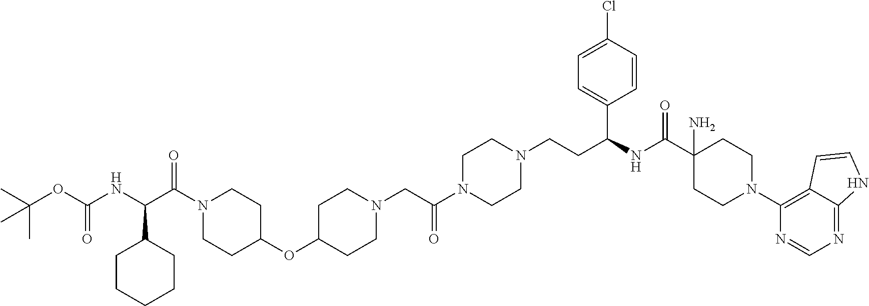 [SMILES: CC(C)(C)OC(=O)N[C@@H](C(=O)N1CCC(OC2CCN(CC(=O)N3CCN(CC[C@H](NC(=O)C4(N)CCN(c5ncnc6[nH]ccc56)CC4)c4ccc(Cl)cc4)CC3)CC2)CC1)C1CCCCC1] |
| I-313 | 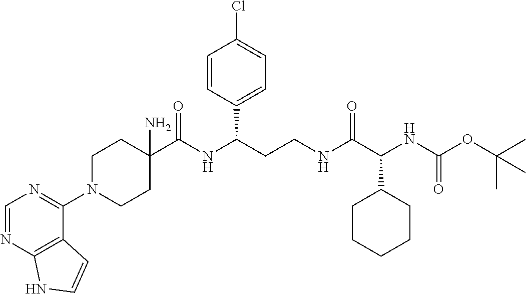 [SMILES: CC(C)(C)OC(=O)N[C@@H](C(=O)NCC[C@H](NC(=O)C1(N)CCN(c2ncnc3[nH]ccc23)CC1)c1ccc(Cl)cc1)C1CCCCC1] |
| I-314 | 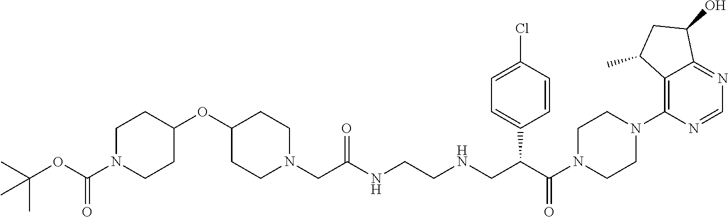 [SMILES: C[C@@H]1C[C@@H](O)c2ncnc(N3CCN(C(=O)[C@H](CNCCNC(=O)CN4CCC(OC5CCN(C(=O)OC(C)(C)C)CC5)CC4)c4ccc(Cl)cc4)CC3)c21] |
| I-315 | 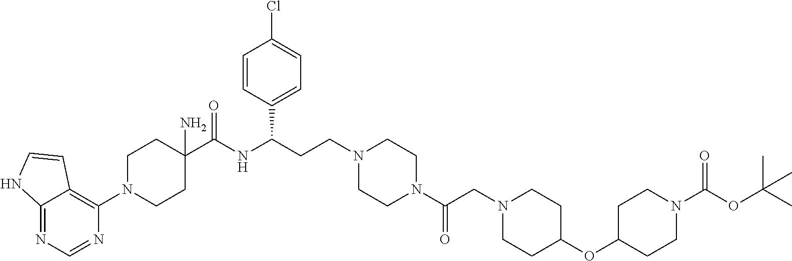 [SMILES: CC(C)(C)OC(=O)N1CCC(OC2CCN(CC(=O)N3CCN(CC[C@H](NC(=O)C4(N)CCN(c5ncnc6[nH]ccc56)CC4)c4ccc(Cl)cc4)CC3)CC2)CC1] |
| I-316 | 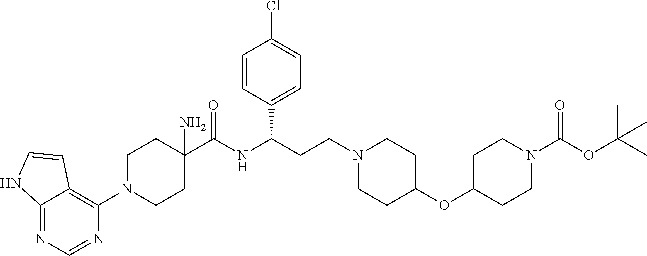 [SMILES: CC(C)(C)OC(=O)N1CCC(OC2CCN(CC[C@H](NC(=O)C3(N)CCN(c4ncnc5[nH]ccc45)CC3)c3ccc(Cl)cc3)CC2)CC1] |
| I-317 | 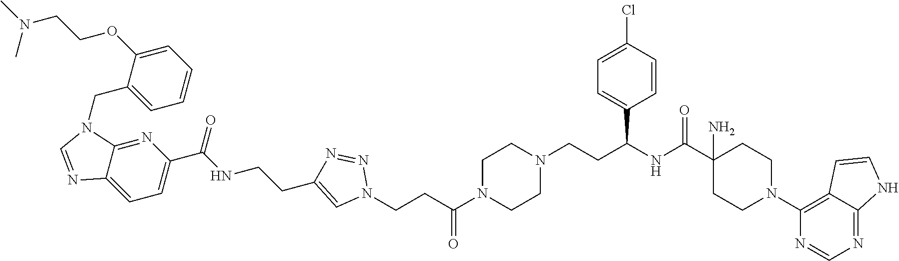 [SMILES: CN(C)CCOc1ccccc1Cn1cnc2ccc(C(=O)NCCc3cn(CCC(=O)N4CCN(CC[C@H](NC(=O)C5(N)CCN(c6ncnc7[nH]ccc67)CC5)c5ccc(Cl)cc5)CC4)nn3)nc21] |
| I-318 | 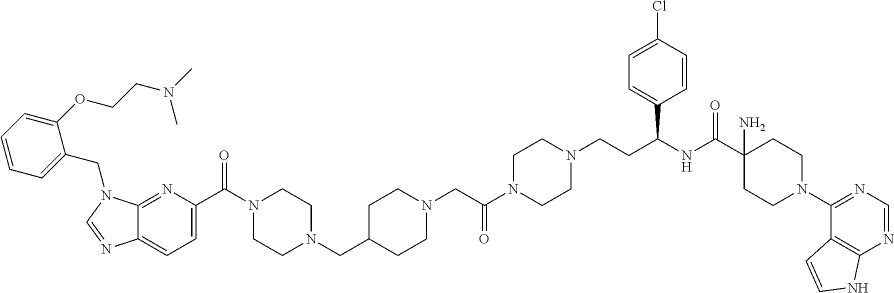 [SMILES: CN(C)CCOc1ccccc1Cn1cnc2ccc(C(=O)N3CCN(CC4CCN(CC(=O)N5CCN(CC[C@H](NC(=O)C6(N)CCN(c7ncnc8[nH]ccc78)CC6)c6ccc(Cl)cc6)CC5)CC4)CC3)nc21] |
| I-319 | 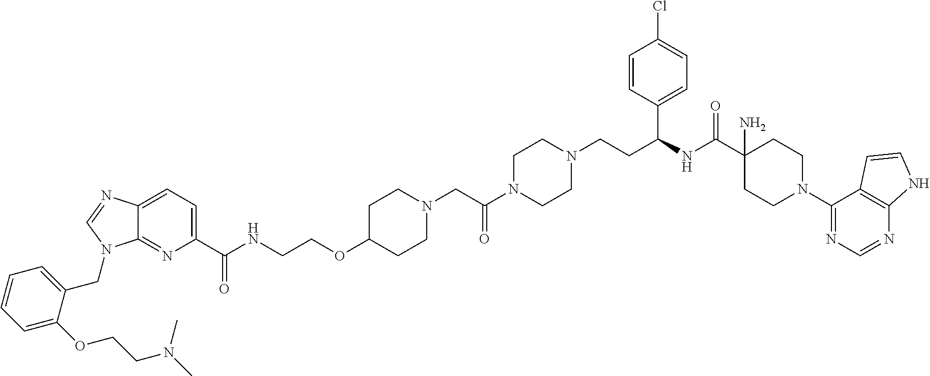 [SMILES: CN(C)CCOc1ccccc1Cn1cnc2ccc(C(=O)NCCOC3CCN(CC(=O)N4CCN(CC[C@H](NC(=O)C5(N)CCN(c6ncnc7[nH]ccc67)CC5)c5ccc(Cl)cc5)CC4)CC3)nc21] |
| I-320 | 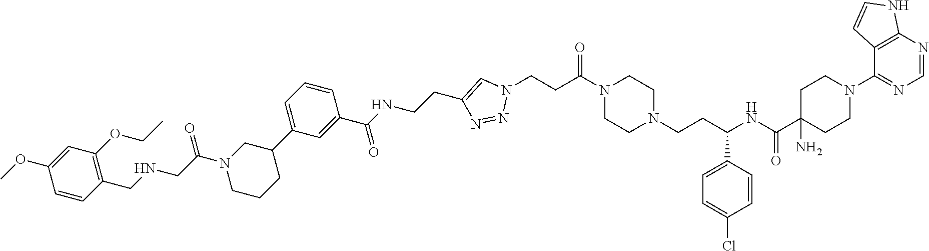 [SMILES: CCOc1cc(OC)ccc1CNCC(=O)N1CCCC(c2cccc(C(=O)NCCc3cn(CCC(=O)N4CCN(CC[C@H](NC(=O)C5(N)CCN(c6ncnc7[nH]ccc67)CC5)c5ccc(Cl)cc5)CC4)nn3)c2)C1] |
| I-321 | 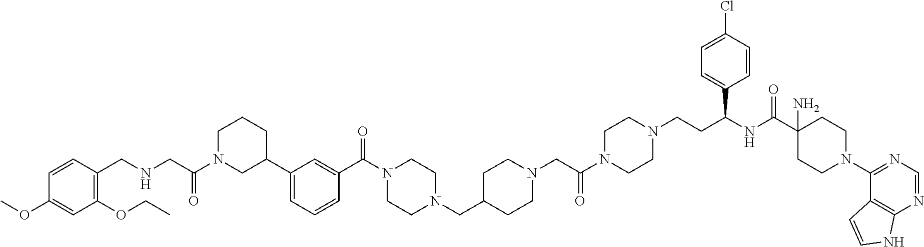 [SMILES: CCOc1cc(OC)ccc1CNCC(=O)N1CCCC(c2cccc(C(=O)N3CCN(CC4CCN(CC(=O)N5CCN(CC[C@H](NC(=O)C6(N)CCN(c7ncnc8[nH]ccc78)CC6)c6ccc(Cl)cc6)CC5)CC4)CC3)c2)C1] |
| I-322 | 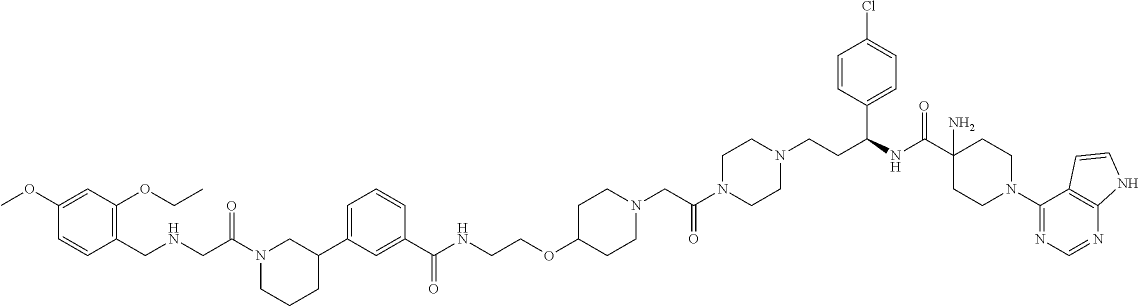 [SMILES: CCOc1cc(OC)ccc1CNCC(=O)N1CCCC(c2cccc(C(=O)NCCOC3CCN(CC(=O)N4CCN(CC[C@H](NC(=O)C5(N)CCN(c6ncnc7[nH]ccc67)CC5)c5ccc(Cl)cc5)CC4)CC3)c2)C1] |
| I-323 | 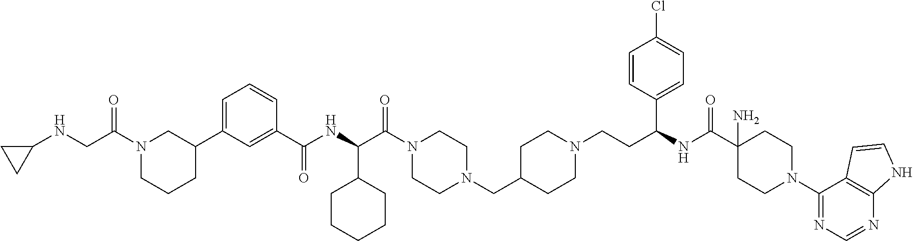 [SMILES: NC1(C(=O)N[C@@H](CCN2CCC(CN3CCN(C(=O)[C@H](NC(=O)c4cccc(C5CCCN(C(=O)CNC6CC6)C5)c4)C4CCCCC4)CC3)CC2)c2ccc(Cl)cc2)CCN(c2ncnc3[nH]ccc23)CC1] |
| I-324 | 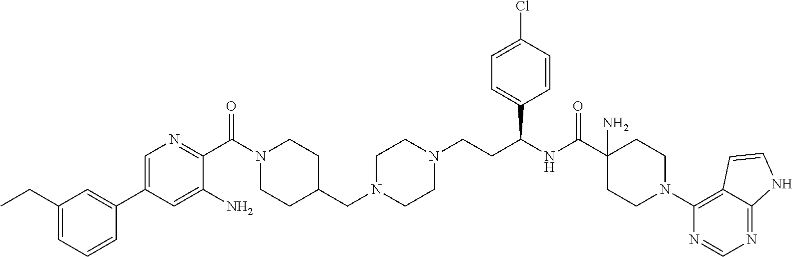 [SMILES: CCc1cccc(-c2cnc(C(=O)N3CCC(CN4CCN(CC[C@H](NC(=O)C5(N)CCN(c6ncnc7[nH]ccc67)CC5)c5ccc(Cl)cc5)CC4)CC3)c(N)c2)c1] |
| I-325 | 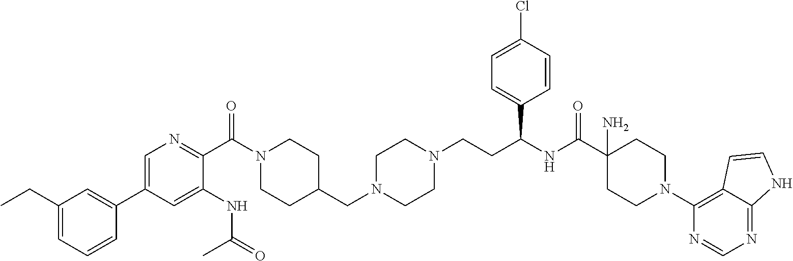 [SMILES: CCc1cccc(-c2cnc(C(=O)N3CCC(CN4CCN(CC[C@H](NC(=O)C5(N)CCN(c6ncnc7[nH]ccc67)CC5)c5ccc(Cl)cc5)CC4)CC3)c(NC(C)=O)c2)c1] |
| I-326 | 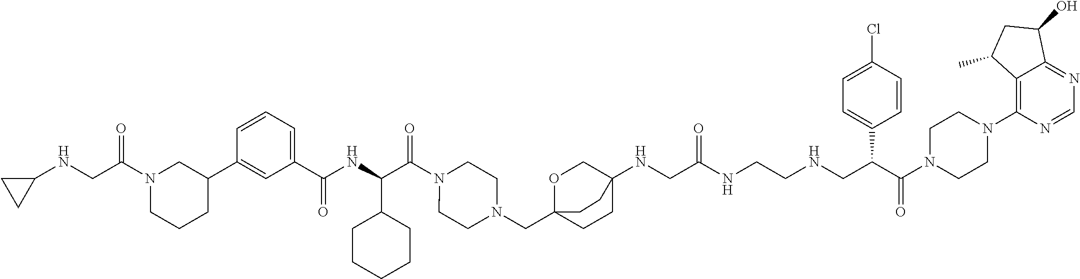 [SMILES: C[C@@H]1C[C@@H](O)c2ncnc(N3CCN(C(=O)[C@H](CNCCNC(=O)CNC45CCC(CN6CCN(C(=O)[C@H](NC(=O)c7cccc(C8CCCN(C(=O)CNC9CC9)C8)c7)C7CCCCC7)CC6)(CC4)OC5)c4ccc(Cl)cc4)CC3)c21] |
| I-327 | 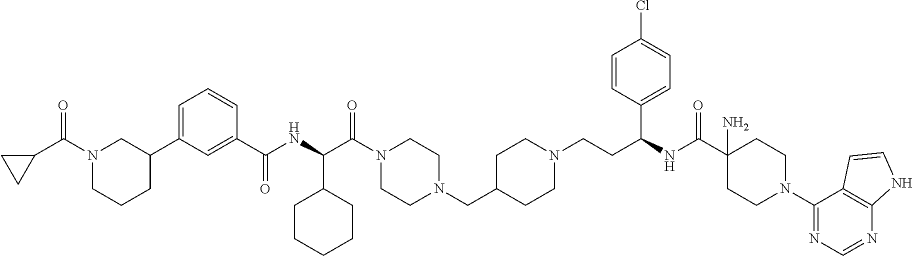 [SMILES: NC1(C(=O)N[C@@H](CCN2CCC(CN3CCN(C(=O)[C@H](NC(=O)c4cccc(C5CCCN(C(=O)C6CC6)C5)c4)C4CCCCC4)CC3)CC2)c2ccc(Cl)cc2)CCN(c2ncnc3[nH]ccc23)CC1] |
| I-328 | 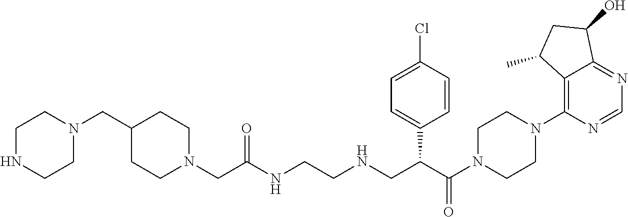 [SMILES: C[C@@H]1C[C@@H](O)c2ncnc(N3CCN(C(=O)[C@H](CNCCNC(=O)CN4CCC(CN5CCNCC5)CC4)c4ccc(Cl)cc4)CC3)c21] |
| I-329 | 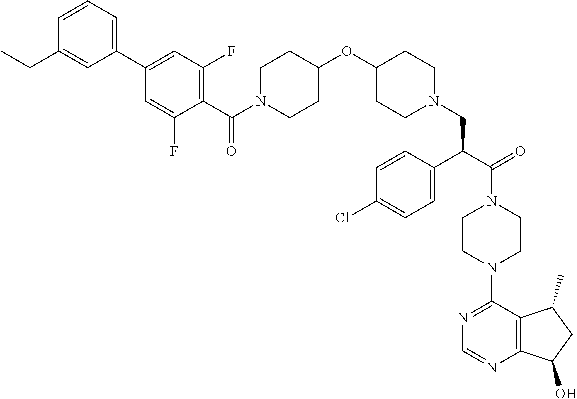 [SMILES: CCc1cccc(-c2cc(F)c(C(=O)N3CCC(OC4CCN(C[C@@H](C(=O)N5CCN(c6ncnc7c6[C@H](C)C[C@H]7O)CC5)c5ccc(Cl)cc5)CC4)CC3)c(F)c2)c1] |
| I-330 | 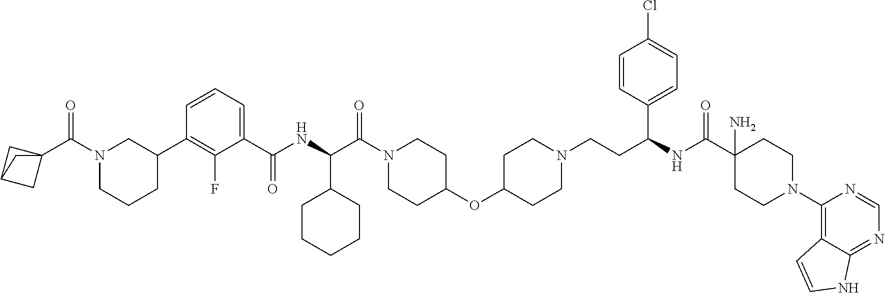 [SMILES: NC1(C(=O)N[C@@H](CCN2CCC(OC3CCN(C(=O)[C@H](NC(=O)c4cccc(C5CCCN(C(=O)C67CC(C6)C7)C5)c4F)C4CCCCC4)CC3)CC2)c2ccc(Cl)cc2)CCN(c2ncnc3[nH]ccc23)CC1] |
| I-331 | 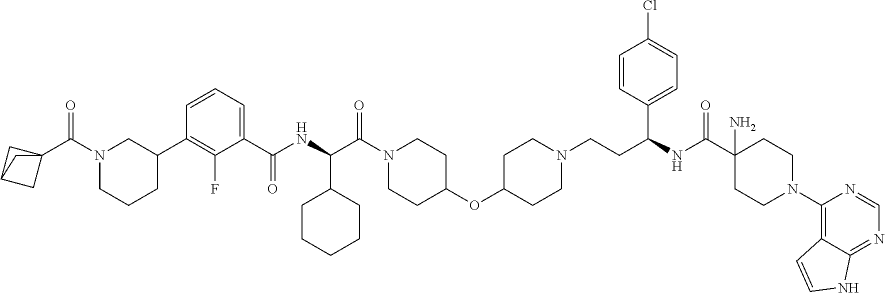 [SMILES: NC1(C(=O)N[C@@H](CCN2CCC(OC3CCN(C(=O)[C@H](NC(=O)c4cccc(C5CCCN(C(=O)C67CC(C6)C7)C5)c4F)C4CCCCC4)CC3)CC2)c2ccc(Cl)cc2)CCN(c2ncnc3[nH]ccc23)CC1] |
| I-332 | 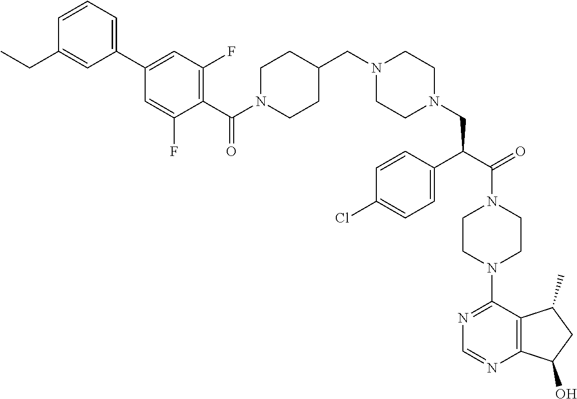 [SMILES: CCc1cccc(-c2cc(F)c(C(=O)N3CCC(CN4CCN(C[C@@H](C(=O)N5CCN(c6ncnc7c6[C@H](C)C[C@H]7O)CC5)c5ccc(Cl)cc5)CC4)CC3)c(F)c2)c1] |
| I-333 | 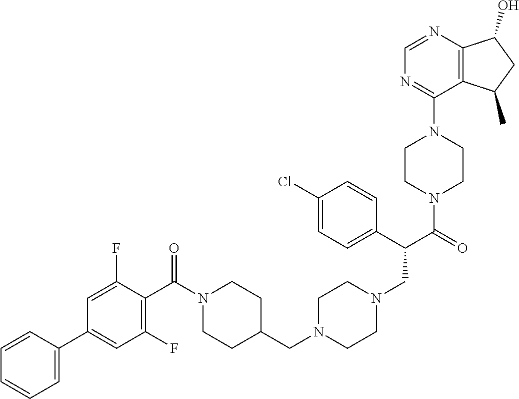 [SMILES: C[C@@H]1C[C@@H](O)c2ncnc(N3CCN(C(=O)[C@H](CN4CCN(CC5CCN(C(=O)c6c(F)cc(-c7ccccc7)cc6F)CC5)CC4)c4ccc(Cl)cc4)CC3)c21] |
| I-334 | 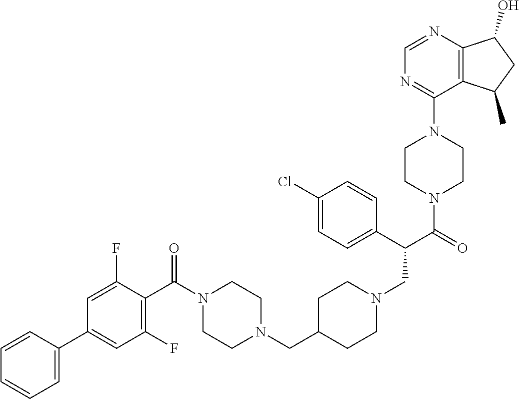 [SMILES: C[C@@H]1C[C@@H](O)c2ncnc(N3CCN(C(=O)[C@H](CN4CCC(CN5CCN(C(=O)c6c(F)cc(-c7ccccc7)cc6F)CC5)CC4)c4ccc(Cl)cc4)CC3)c21] |
| I-335 | 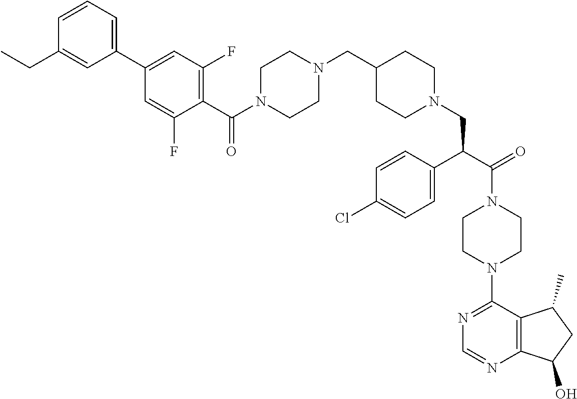 [SMILES: CCc1cccc(-c2cc(F)c(C(=O)N3CCN(CC4CCN(C[C@@H](C(=O)N5CCN(c6ncnc7c6[C@H](C)C[C@H]7O)CC5)c5ccc(Cl)cc5)CC4)CC3)c(F)c2)c1] |
| I-336 | 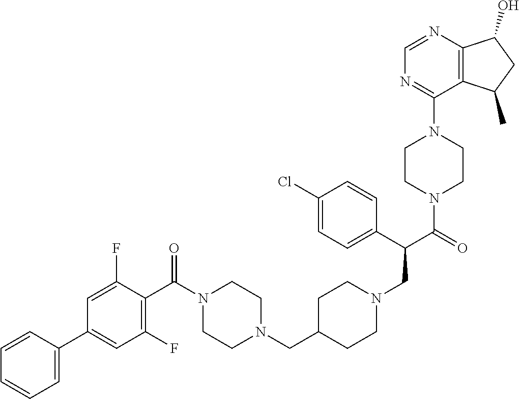 [SMILES: C[C@@H]1C[C@@H](O)c2ncnc(N3CCN(C(=O)[C@@H](CN4CCC(CN5CCN(C(=O)c6c(F)cc(-c7ccccc7)cc6F)CC5)CC4)c4ccc(Cl)cc4)CC3)c21] |
| I-337 | 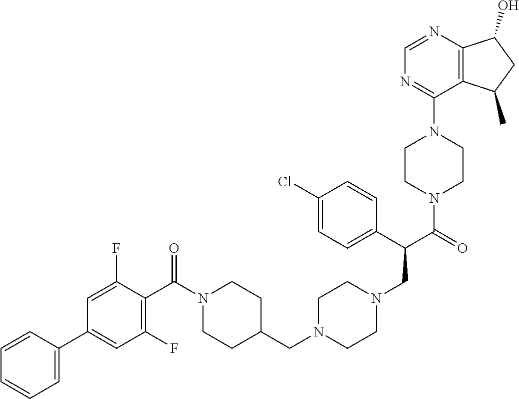 [SMILES: C[C@@H]1C[C@@H](O)c2ncnc(N3CCN(C(=O)[C@@H](CN4CCN(CC5CCN(C(=O)c6c(F)cc(-c7ccccc7)cc6F)CC5)CC4)c4ccc(Cl)cc4)CC3)c21] |
| I-338 | 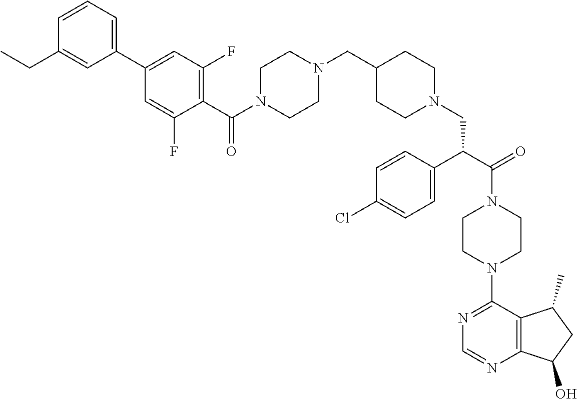 [SMILES: CCc1cccc(-c2cc(F)c(C(=O)N3CCN(CC4CCN(C[C@H](C(=O)N5CCN(c6ncnc7c6[C@H](C)C[C@H]7O)CC5)c5ccc(Cl)cc5)CC4)CC3)c(F)c2)c1] |
| I-339 | 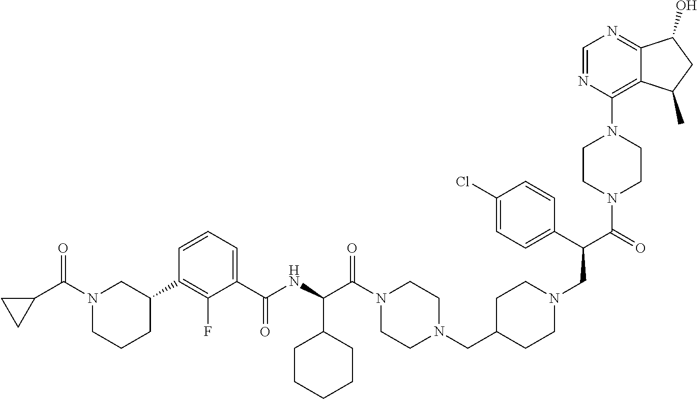 [SMILES: C[C@@H]1C[C@@H](O)c2ncnc(N3CCN(C(=O)[C@@H](CN4CCC(CN5CCN(C(=O)[C@H](NC(=O)c6cccc([C@@H]7CCCN(C(=O)C8CC8)C7)c6F)C6CCCCC6)CC5)CC4)c4ccc(Cl)cc4)CC3)c21] |
| I-340 | 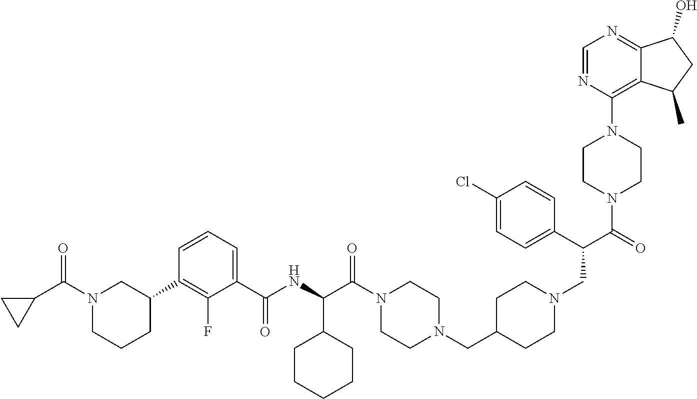 [SMILES: C[C@@H]1C[C@@H](O)c2ncnc(N3CCN(C(=O)[C@H](CN4CCC(CN5CCN(C(=O)[C@H](NC(=O)c6cccc([C@@H]7CCCN(C(=O)C8CC8)C7)c6F)C6CCCCC6)CC5)CC4)c4ccc(Cl)cc4)CC3)c21] |

In some embodiments, the present invention provides a compound set forth in Table 1, above, or a pharmaceutically acceptable salt thereof.

### 4. General Methods of Providing the Present Compounds

The compounds of this invention may be prepared or isolated in general by synthetic and/or semi-synthetic methods known to those skilled in the art for analogous compounds and by methods described in detail in the Examples, herein.

In the Schemes below, where a particular protecting group, leaving group, or transformation condition is depicted, one of ordinary skill in the art will appreciate that other protecting groups, leaving groups, and transformation conditions are also suitable and are contemplated. Such groups and transformations are described in detail in March's Advanced Organic Chemistry: Reactions, Mechanisms, and Structure, M. B. Smith and J. March 5^{th} Edition, John Wiley & Sons, 2001, Comprehensive Organic Transformations, R. C. Larock, 2^{nd} Edition. John Wiley & Sons, 1999, and Protecting Groups in Organic Synthesis, T. W. Greene and P. G. M. Wuts, 3^{rd} edition. John Wiley & Sons, 1999, the entirety of each of which is hereby incorporated herein by reference.

As used herein, the phrase “oxygen protecting group” includes, for example, carbonyl protecting groups, hydroxyl protecting groups, etc. Hydroxyl protecting groups are well known in the art and include those described in detail in Protecting Groups in Organic Synthesis, T. W. Greene and P. G. M. Wuts, 3^{rd} edition, John Wiley & Sons, 1999, the entirety of each of which is herein incorporated by reference. Examples of suitable hydroxyl protecting groups include, but are not limited to, esters, allyl ethers, ethers, silyl ethers, alkyl ethers, arylalkyl ethers, and alkoxyalkyl ethers. Examples of such esters include formates, acetates, carbonates, and sulfonates. Specific examples include formate, benzoyl formate, chloroacetate, trifluoroacetate, methoxyacetate, triphenylmethoxyacetate, p-chlorophenoxyacetate, 3-phenylpropionate, 4-oxopentanoate, 4,4-(ethylenedithio)pentanoate, pivaloate (trimethylacetyl), crotonate, 4-methoxy-crotonate, benzoate, p-benylbenzoate, 2,4,6-trimethylbenzoate, carbonates such as methyl, 9-fluorenylmethyl, ethyl, 2,2,2-trichloroethyl, 2-(trimethylsilyl)ethyl, 2-(phenylsulfonyl)ethyl, vinyl, allyl, and p-nitrobenzyl. Examples of such silyl ethers include trimethylsilyl, triethylsilyl, t-butyldimethylsilyl, t-butyldiphenylsilyl, triisopropylsilyl, and other trialkylsilyl ethers. Alkyl ethers include methyl, benzyl, p-methoxybenzyl, 3,4-dimethoxybenzyl, trityl, t-butyl, allyl, and allyloxycarbonyl ethers or derivatives. Alkoxyalkyl ethers include acetals such as methoxymethyl, methylthiomethyl, (2-methoxyethoxy)methyl, benzyloxymethyl, beta-(trimethylsilyl)ethoxymethyl, and tetrahydropyranyl ethers. Examples of arylalkyl ethers include benzyl, p-methoxybenzyl (MPM), 3,4-dimethoxybenzyl, O-nitrobenzyl, p-nitrobenzyl, p-halobenzyl, 2,6-dichlorobenzyl, p-cyanobenzyl, and 2- and 4-picolyl.

Amino protecting groups are well known in the art and include those described in detail in Protecting Groups in Organic Synthesis, T. W. Greene and P. G. M. Wuts, 3^{rd} edition, John Wiley & Sons, 1999, the entirety of each of which is herein incorporated by reference. Suitable amino protecting groups include, but are not limited to, aralkylamines, carbamates, cyclic imides, allyl amines, amides, and the like. Examples of such groups include t-butyloxycarbonyl (BOC), ethyloxycarbonyl, methyloxycarbonyl, trichloroethyloxycarbonyl, allyloxycarbonyl (Alloc), benzyloxocarbonyl (CBZ), allyl, phthalimide, benzyl (Bn), fluorenylmethylcarbonyl (Fmoc), formyl, acetyl, chloroacetyl, dichloroacetyl, trichloroacetyl, phenylacetyl, trifluoroacetyl, benzoyl, and the like.

In the schemes below, where a provided compound is formed having a reactive moiety (e.g., amine, alcohol, etc.), it is not shown but it is generally appreciated and well known by those having ordinary skill in the art that the reactivity of said reactive moiety may be masked by employing a suitable protecting group that can thereafter be removed in situ or during a separate synthetic step.

In some embodiments, compounds of the present invention are generally prepared according to Scheme 1 set forth below:

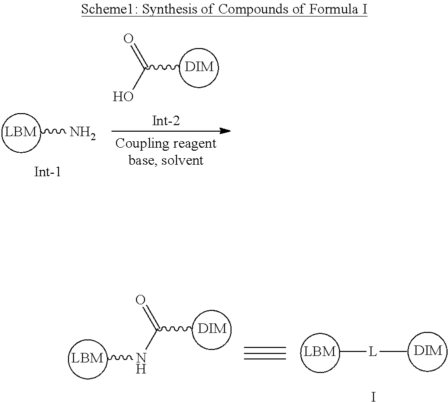

As depicted in Scheme 1, above, amine Int-1 is coupled to acid Int-2 using a coupling reagent (e.g., EDCI) in the presence of the base (e.g., NMM) in a solvent (e.g., DMF) to form a compound of formula I with a linker comprising an amide bond. The squiggly bond, [symbol] , represents the portion of the linker between PBM and the terminal amino group of Int-1 or the portion of the linker between DIM and the terminal carboxyl group of Int-2, respectively. The amide bond can be formed using coupling reagents known in the art such as, but not limited to DCC, DIC, EDC, HATU, HBTU, HCTU, PyAOP, PyBOP, PyBrOP, BOP, BOP—Cl, DEPBT, T3P, TATU, TBTU, TNTU, TOTU, TPTU, TSTU, or TDBTU.

In some embodiments, compounds of the present invention are generally prepared according to Scheme 2 set forth below:

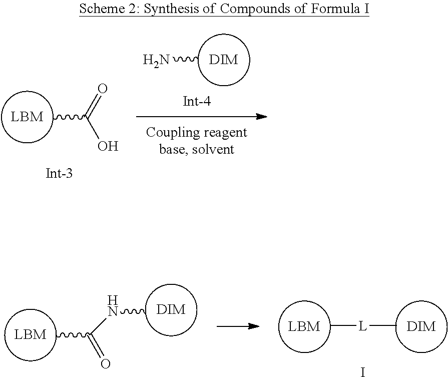

As depicted in Scheme 2, above, acid Int-3 is coupled to amine Int-4 using a coupling reagent (e.g., EDCI) in the presence of the base (e.g., NMM) in a solvent (e.g., DMF) to form a compound of formula I with a linker comprising an amide bond. The squiggly bond, [symbol] , represents the portion of the linker between PBM and the terminal carboxyl group of Int-3 or the portion of the linker between DIM and the terminal amino group of Int-4, respectively. The amide bond can be formed using coupling reagents known in the art such as, but not limited to DCC, DIC, EDC, HATU, HBTU, HCTU, PyAOP, PyBOP, PyBrOP, BOP, BOP—Cl, DEPBT, T3P, TATU, TBTU, TNTU, TOTU, TPTU, TSTU, or TDBTU.

One of skill in the art will appreciate that various functional groups present in compounds of the invention such as aliphatic groups, alcohols, carboxylic acids, esters, amides, aldehydes, halogens and nitriles can be interconverted by techniques well known in the art including, but not limited to reduction, oxidation, esterification, hydrolysis, partial oxidation, partial reduction, halogenation, dehydration, partial hydration, and hydration. “March's Advanced Organic Chemistry”, 5th Ed., Ed.: Smith, M. B, and March, J., John Wiley & Sons, New York: 2001, the entirety of which is incorporated herein by reference. Such interconversions may require one or more of the aforementioned techniques, and certain methods for synthesizing compounds of the invention are described below in the Exemplification.

### 5. Uses, Formulation and Administration

### Pharmaceutically Acceptable Compositions

In some embodiments, the invention provides a composition comprising a compound of this invention or a pharmaceutically acceptable derivative thereof and a pharmaceutically acceptable carrier, adjuvant, or vehicle. In some embodiments, the amount of compound in compositions of this invention is such that it is effective to measurably inhibit an Akt protein, or a mutant thereof, in a biological sample or in a patient. In some embodiments, a composition of this invention is formulated for administration to a patient in need of such composition. In some embodiments, a composition of this invention is formulated for oral administration to a patient.

The term “patient,” as used herein, means an animal, preferably a mammal, and most preferably a human.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A “pharmaceutically acceptable derivative” means any non-toxic salt, ester, salt of an ester or other derivative of a compound of this invention that, upon administration to a recipient, is capable of providing, either directly or indirectly; a compound of this invention or an inhibitorily active metabolite or residue thereof.

As used herein, the term “inhibitorily active metabolite or residue thereof” means that a metabolite or residue thereof is also an inhibitor of an Akt protein, or a mutant thereof.

Compositions of the present invention may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Pharmaceutically acceptable compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, pharmaceutically acceptable compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, provided pharmaceutically acceptable compositions may be formulated in a suitable ointment containing the active component suspended or dissolved in one or more carriers. Carriers for topical administration of compounds of this invention include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, provided pharmaceutically acceptable compositions may be formulated as micronized suspensions in isotonic, pH adjusted sterile saline, or, preferably; as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

Pharmaceutically acceptable compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Most preferably: pharmaceutically acceptable compositions of this invention are formulated for oral administration. Such formulations may be administered with or without food. In some embodiments, pharmaceutically acceptable compositions of this invention are administered without food. In other embodiments, pharmaceutically acceptable compositions of this invention are administered with food.

The amount of compounds of the present invention that may be combined with the carrier materials to produce a composition in a single dosage form will vary depending upon the host treated, the particular mode of administration. Preferably, provided compositions should be formulated so that a dosage of between 0.01-100 mg/kg body weight/day of the compound can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present invention in the composition will also depend upon the particular compound in the composition.

### Uses of Compounds and Pharmaceutically Acceptable Compositions

Compounds and compositions described herein are generally useful for the inhibition of kinase activity of one or more enzymes.

As used herein, the terms “AKT-mediated”, “AKT1-mediated”, “AKT2-mediated”, and/or “AKT3-mediated” disorders, diseases, and/or conditions as used herein means any disease or other deleterious condition in which one or more of AKT1, AKT2, and/or AKT3, or a mutant thereof (e.g., E17K AKT), are known to play a role. Accordingly, another embodiment of the present invention relates to treating or lessening the severity of one or more diseases in which one or more of AKT1, AKT2, and/or AKT3, or a mutant thereof, are known to play a role.

Compounds of the present disclosure can inhibit AKT1, AKT2, or AKT3 and therefore are useful for treating diseases wherein the underlying pathology is, wholly or partially, mediated by AKT1, AKT2, and/or AKT3. Such diseases include cancer and other diseases with proliferation disorder. In some embodiments, the present disclosure provides treatment of an individual or a patient in vivo using a provided compound or a pharmaceutically acceptable salt thereof such that growth of cancerous tumors is inhibited. A provided compound or a pharmaceutically acceptable salt thereof can be used to inhibit the growth of cancerous tumors with aberrations that activate AKT1, AKT2, and/or AKT3 activity. These include, but not limited to, disease (e.g., cancers) that are characterized by amplification or overexpression of AKT1, AKT2, and/or AKT3 such as ovarian cancer, uterine carcinosarcoma and breast cancer and p27 inactivation such as breast cancer and melanomas. Accordingly, in some embodiments of the methods, the patient has been previously determined to have an amplification of the Akt gene in a biological sample obtained from the human subject that is higher than a control expression level of Akt. Alternatively, a provided compound or a pharmaceutically acceptable salt thereof can be used in conjunction with other agents or standard cancer treatments, as described below. In one embodiment, the present disclosure provides a method for inhibiting growth of tumor cells in vitro. The method includes contacting the tumor cells in vitro with a provided compound or a pharmaceutically acceptable salt thereof. In another embodiment, the present disclosure provides a method for inhibiting growth of tumor cells with Akt amplification and overexpression in an individual or a patient. The method includes administering to the individual or patient in need thereof a therapeutically effective amount of a provided compound or a pharmaceutically acceptable salt thereof.

In some embodiments, compounds of the present disclosure can inhibit E17K AKT.

In some embodiments, provided herein is a method of inhibiting Akt, comprising contacting the Akt with a provided compound or a pharmaceutically acceptable salt thereof. In some embodiments, provided herein is a method of inhibiting Akt in a patient, comprising administering to the patient a provided compound or a pharmaceutically acceptable salt thereof.

In some embodiments, provided herein is a method for treating cancer. In some embodiments, the cancer is associated with irregularities in the activity of Akt and/or downstream cellular targets of Akt. Such cancers include, but are not limited to, ovarian, pancreatic, breast and prostate cancer, as well as cancers (including glioblastoma) where the tumor suppressor PTEN is mutated as described in: Cheng et al., Proc. Natl. Acad. Sci. (1992) 89:9267-9271; Cheng et al., Proc. Natl. Acad. Sci. (1996) 93:3636-3641; Bellacosa et al., Int. J. Cancer (1995) 64:280-285; Nakatani et al., J. Biol Chem. (1999) 274:21528-21532; Graff. Expert. Opin. Ther. Targets (2002) 6 (1): 103-113; and Yamadaand Araki. J. Cell Science. (2001) 114:2375-2382; Mischel and Cloughesy, Brain Pathol. (2003) 13 (1): 52-61). Cancers where Akt itself is activated by gene amplification or mutations may also be treated by the compounds. Human breast, colorectal and ovarian cancers where a somatic mutation in a pleckstrin homology domain (PH) of AKT1 (E17K mutant; the glutamic acid (E) at position 17 of the amino acid sequence of the PH domain of AKT1 is replaced by a lysine (K)) is reported (Carpten et al. Nature 448:439-444 (2007), the entirety of each of which is hereby incorporated herein by reference.). The method includes administering to a patient (in need thereof), a therapeutically effective amount of a provided compound or a pharmaceutically acceptable salt thereof. In another embodiment, the cancer is characterized by amplification or overexpression of Akt. In some embodiments, the cancer is ovarian cancer or breast cancer, characterized by amplification or overexpression of Akt.

In some embodiments, provided herein is a method of treating a disease or disorder associated with Akt in a patient, comprising administering to the patient a therapeutically effective amount of a provided compound or a pharmaceutically acceptable salt thereof. In some embodiments, the disease or disorder associated with Akt is associated with an amplification of the Akt gene and/or overexpression of Akt.

In some embodiments, the disease or disorder associated with Akt is lung squamous cell carcinoma, lung adenocarcinoma, pancreatic adenocarcinoma, breast invasive carcinoma, uterine carcinosarcoma, ovarian serous cystadenocarcinoma, stomach adenocarcinoma, esophageal carcinoma, bladder urothelial carcinoma, mesothelioma, or sarcoma.

In some embodiments, the disease or disorder associated with Akt is lung adenocarcinoma, breast invasive carcinoma, uterine carcinosarcoma, ovarian serous cystadenocarcinoma, or stomach adenocarcinoma.

In some embodiments, the disease or disorder associated with Akt is an adenocarcinoma, carcinoma, or cystadenocarcinoma.

In some embodiments, the disease or disorder associated with Akt is uterine cancer, ovarian cancer, stomach cancer, esophageal cancer, lung cancer, bladder cancer, pancreatic cancer, or breast cancer. In some embodiments, the disease or disorder associated with Akt is a cancer.

In some embodiments, the cancer is characterized by amplification or overexpression of Akt. In some embodiments, the cancer is ovarian cancer or breast cancer, characterized by amplification or overexpression of Akt.

Examples of cancers that are treatable using the compounds of the present disclosure include, but are not limited to, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, endometrial cancer, carcinoma of the cervix, carcinoma of the vagina, carcinoma of the vulva, Hodgkin's Disease, non-Hodgkin's lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemias including acute myeloid leukemia, chronic myeloid leukemia, acute lymphoblastic leukemia, chronic lymphocytic leukemia, solid tumors of childhood, lymphocytic lymphoma, cancer of the bladder, cancer of the kidney or urethra, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers including those induced by asbestos, and combinations of said cancers. The compounds of the present disclosure are also useful for the treatment of metastatic cancers.

In some embodiments, cancers treatable with compounds of the present disclosure include melanoma (e.g., metastatic malignant melanoma, BRAF and HSP90 inhibition-resistant melanoma), renal cancer (e.g., clear cell carcinoma), prostate cancer (e.g., hormone refractory prostate adenocarcinoma), breast cancer, colon cancer, lung cancer (e.g., non-small cell lung cancer and small cell lung cancer), squamous cell head and neck cancer, urothelial cancer (e.g., bladder) and cancers with high microsatellite instability (MSI^{high}). Additionally, the disclosure includes refractory or recurrent malignancies whose growth may be inhibited using the compounds of the disclosure.

In some embodiments, cancers that are treatable using the compounds of the present disclosure include, but are not limited to: Cardiac: sarcoma (angiosarcoma, fibrosarcoma, rhabdomyosarcoma, liposarcoma), myxoma, rhabdomyoma, fibroma, lipoma and teratoma; Lung: non-small cell lung, bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma), colon, colorectal, rectal; Genitourinary tract: kidney (adenocarcinoma, Wilm's tumor [nephroblastoma], lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarciaoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma [pincaloma], glioblastoma multiform, oligodendroglioma, schwannoma, retinoblastoma, congenital tumors), spinal cord neurofibroma, meningioma, glioma, sarcoma); Gynecological: uterus (endometrial carcinoma), cervix (cervical carcinoma, pre-tumor cervical dysplasia), ovaries (ovarian carcinoma [serous cystadenocarcinoma, mucinous cystadenocarcinoma, unclassified carcinoma], granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma); Hematologic: blood (myeloid leukemia [acute and chronic], acute lymphoblastic leukemia, chronic lymphocytic leukemia, myeloproliferative diseases, multiple myeloma, myelodysplastic syndrome), Hodgkin's disease, non-Hodgkin's lymphoma [malignant lymphoma]; Skin: malignant melanoma, basal cell carcinoma, squamous cell carcinoma, Karposi's sarcoma, moles dysplastic nevi, lipoma, angioma, dermatofibroma, keloids, psoriasis; and Adrenal glands: neuroblastoma. Thus, the term “cancerous cell” as provided herein, includes a cell afflicted by any one of the above-identified conditions.

In some embodiments, cancers that are treatable using the compounds of the present disclosure include, but are not limited to, solid tumors (e.g., prostate cancer, colon cancer, esophageal cancer, endometrial cancer, ovarian cancer, uterine cancer, renal cancer, hepatic cancer, pancreatic cancer, gastric cancer, breast cancer, lung cancer, cancers of the head and neck, thyroid cancer, glioblastoma, sarcoma, bladder cancer, etc.), hematological cancers (e.g., lymphoma, leukemia such as acute lymphoblastic leukemia (ALL), acute myelogenous leukemia (AML), chronic lymphocytic leukemia (CLL), chronic myelogenous leukemia (CML), DLBCL, mantle cell lymphoma, Non-Hodgkin lymphoma (including relapsed or refractory NHL and recurrent follicular), Hodgkin lymphoma or multiple myeloma) and combinations of said cancers.

In some embodiments, cancers that are treatable using the compounds of the present disclosure include, but are not limited to, cholangiocarcinoma, bile duct cancer, triple negative breast cancer, rhabdomyosarcoma, small cell lung cancer, leiomyosarcoma, hepatocellular carcinoma, Ewing's sarcoma, brain cancer, brain tumor, astrocytoma, neuroblastoma, neurofibroma, basal cell carcinoma, chondrosarcoma, epithelioid sarcoma, eye cancer, Fallopian tube cancer, gastrointestinal cancer, gastrointestinal stromal tumors, hairy cell leukemia, intestinal cancer, islet cell cancer, oral cancer, mouth cancer, throat cancer, laryngeal cancer, lip cancer, mesothelioma, neck cancer, nasal cavity cancer, ocular cancer, ocular melanoma, pelvic cancer, rectal cancer, renal cell carcinoma, salivary gland cancer, sinus cancer, spinal cancer, tongue cancer, tubular carcinoma, urethral cancer, and ureteral cancer.

In some embodiments, the compounds of the present disclosure can be used to treat sickle cell disease and sickle cell anemia.

In some embodiments, diseases and indications that are treatable using the compounds of the present disclosure include, but are not limited to hematological cancers, sarcomas, lung cancers, gastrointestinal cancers, genitourinary tract cancers, liver cancers, bone cancers, nervous system cancers, gynecological cancers, and skin cancers.

Exemplary hematological cancers include lymphomas and leukemias such as acute lymphoblastic leukemia (ALL), acute myelogenous leukemia (AML), acute promyelocytic leukemia (APL), chronic lymphocytic leukemia (CLL), chronic myelogenous leukemia (CML), diffuse large B-cell lymphoma (DLBCL), mantle cell lymphoma, Non-Hodgkin lymphoma (including relapsed or refractory NHL and recurrent follicular), Hodgkin lymphoma, myeloproliferative diseases (e.g., primary myelofibrosis (PMF), polycythemia vera (PV), and essential thrombocytosis (ET)), myelodysplasia syndrome (MDS), T-cell acute lymphoblastic lymphoma (T-ALL) and multiple myeloma (MM).

Exemplary sarcomas include chondrosarcoma, Ewing's sarcoma, osteosarcoma, rhabdomyosarcoma, angiosarcoma, fibrosarcoma, liposarcoma, myxoma, rhabdomyoma, rhabdosarcoma, fibroma, lipoma, harmatoma, and teratoma.

Exemplary lung cancers include non-small cell lung cancer (NSCLC), small cell lung cancer (SCLC), bronchogenic carcinoma, squamous cell, undifferentiated small cell, undifferentiated large cell, adenocarcinoma, alveolar (bronchiolar) carcinoma, bronchial adenoma, chondromatous hamartoma, and mesothelioma.

Exemplary gastrointestinal cancers include cancers of the esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma), and colorectal cancer.

Exemplary genitourinary tract cancers include cancers of the kidney (adenocarcinoma, Wilm's tumor [nephroblastoma]), bladder and urethra (squamous cell carcinoma, transitional cell carcinoma, adenocarcinoma), prostate (adenocarcinoma, sarcoma), and testis (seminoma, teratoma, embryonal carcinoma, teratocarcinoma, choriocarcinoma, sarcoma, interstitial cell carcinoma, fibroma, fibroadenoma, adenomatoid tumors, lipoma).

Exemplary liver cancers include hepatoma (hepatocellular carcinoma), cholangiocarcinoma, hepatoblastoma, angiosarcoma, hepatocellular adenoma, and hemangioma.

Exemplary bone cancers include, for example, osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma, and giant cell tumors

Exemplary nervous system cancers include cancers of the skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma, glioblastoma multiform, oligodendroglioma, schwannoma, retinoblastoma, congenital tumors), and spinal cord (neurofibroma, meningioma, glioma, sarcoma), as well as neuroblastoma and Lhermitte-Duclos disease.

Exemplary gynecological cancers include cancers of the uterus (endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), and fallopian tubes (carcinoma).

Exemplary skin cancers include melanoma, basal cell carcinoma, Merkel cell carcinoma, squamous cell carcinoma, Kaposi's sarcoma, moles dysplastic nevi, lipoma, angioma, dermatofibroma, and keloids. In some embodiments, diseases and indications that are treatable using the compounds of the present disclosure include, but are not limited to, sickle cell disease (e.g., sickle cell anemia), triple-negative breast cancer (TNBC), myelodysplastic syndromes, testicular cancer, bile duct cancer, esophageal cancer, and urothelial carcinoma.

In some embodiments, the method comprises killing one or more cancer cells in a patient with cancer. As such, the methods of the disclosure may be used to treat a tumor derived from a neoplasm or a cancer. In some embodiments, the tumor is harboring a mutation. In some embodiments, the mutation is a AKTE17K mutation. In some embodiments, the tumor is harboring PTEN inactivation. In some embodiments, the tumor is harboring one or more PI3K pathway mutations. In some embodiments, the tumor is harboring one or more KRAS pathway mutations.

In some embodiments, the cancer is HR+/HER breast cancer. In some embodiments, the cancer is triple negative breast cancer. In some embodiments, the cancer is HER2-positive breast cancer.

It is believed that a provided compound or a pharmaceutically acceptable salt thereof may possess satisfactory pharmacological profile and promising biopharmaceutical properties, such as toxicological profile, metabolism and pharmacokinetic properties, solubility; and permeability. It will be understood that determination of appropriate biopharmaceutical properties is within the knowledge of a person skilled in the art, e.g., determination of cytotoxicity in cells or inhibition of certain targets or channels to determine potential toxicity.

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

The terms “individual” or “patient.” used interchangeably, refer to any animal, including mammals, preferably mice, rats, other rodents, rabbits, dogs, cats, swine, cattle, sheep, horses, or primates, and most preferably humans.

The phrase “therapeutically effective amount” refers to the amount of active compound or pharmaceutical agent that elicits the biological or medicinal response in a tissue, system, animal, individual or human that is being sought by a researcher, veterinarian, medical doctor or other clinician.

In some embodiments, the compounds of the invention are useful in preventing or reducing the risk of developing any of the diseases referred to herein; e.g., preventing or reducing the risk of developing a disease, condition or disorder in an individual who may be predisposed to the disease, condition or disorder but does not yet experience or display the pathology or symptomatology of the disease.

In some embodiments, compounds of the present disclosure are useful for treating tumors. In some embodiments, the tumor has a BRAF mutation.

Co-Administration with One or More Other Therapeutic Agent(s)

Depending upon the particular condition, or disease, to be treated, additional therapeutic agents that are normally administered to treat that condition, can also be present in the compositions of this invention. As used herein, additional therapeutic agents that are normally administered to treat a particular disease, or condition, are known as “appropriate for the disease, or condition, being treated.”

In some embodiments, the present invention provides a method of treating a disclosed disease or condition comprising administering to a patient in need thereof an effective amount of a compound disclosed herein or a pharmaceutically acceptable salt thereof and co-administering simultaneously or sequentially an effective amount of one or more additional therapeutic agents, such as those described herein. In some embodiments, the method includes co-administering one additional therapeutic agent. In some embodiments, the method includes co-administering two additional therapeutic agents. In some embodiments, the combination of the disclosed compound and the additional therapeutic agent or agents acts synergistically.

A compound of the current invention can also be used in combination with other types of therapies to treat disease. Diseases characterized by Akt activation that could benefit from administration of the compounds include all forms of cancer, precancerous lesions, cardiovascular disease, rheumatologic disease, pulmonary disease, dermatologic disease, gynecological diseases, vascular disease, neurologic disease, and infectious disease, including bacterial, viral, retroviral, and parasitic diseases. Moreover, these compounds could be utilized to prevent above said diseases.

A compound of the current invention can also be used in combination with known therapeutic processes, for example, the administration of hormones or radiation. In some embodiments, a provided compound is used as a radiosensitizer, especially for the treatment of tumors which exhibit poor sensitivity to radiotherapy.

A compound of the current invention can be administered alone or in combination with one or more other therapeutic compounds, possible combination therapy taking the form of fixed combinations or the administration of a compound of the invention and one or more other therapeutic compounds being staggered or given independently of one another, or the combined administration of fixed combinations and one or more other therapeutic compounds. A compound of the current invention can besides, or in addition, be administered especially for tumor therapy in combination with chemotherapy, radiotherapy; immunotherapy; phototherapy, surgical intervention, or a combination of these. Long-term therapy is equally possible, as is adjuvant therapy in the context of other treatment strategies, as described above. Other possible treatments are therapy to maintain the patient's status after tumor regression, or even chemopreventive therapy; for example in patients at risk.

One or more other therapeutic agent(s) can be administered separately from a compound or composition of the invention, as part of a multiple dosage regimen. Alternatively, one or more other therapeutic agent(s) may be part of a single dosage form, mixed together with a compound of this invention in a single composition. If administered as a multiple dosage regime, one or more other therapeutic agent(s) and a compound or composition of the invention can be administered simultaneously; sequentially or within a period of time from one another, for example within 1, 2, 3, 4, 5, 6, 7, 8, 9, 10, 11, 12, 13, 14, 15, 16, 17, 18, 18, 20, 21, 22, 23, or 24 hours from one another. In some embodiments, one or more other therapeutic agent(s) and a compound or composition of the invention are administered as a multiple dosage regimen within greater than 24 hours apart.

As used herein, the term “combination,” “combined,” and related terms refers to the simultaneous or sequential administration of therapeutic agents in accordance with this invention. For example, a compound of the present invention can be administered with one or more other therapeutic agent(s) simultaneously or sequentially in separate unit dosage forms or together in a single unit dosage form. Accordingly, the present invention provides a single unit dosage form comprising a compound of the current invention, one or more other therapeutic agent(s), and a pharmaceutically acceptable carrier, adjuvant, or vehicle.

The amount of a compound of the invention and one or more other therapeutic agent(s) (in those compositions which comprise an additional therapeutic agent as described above) that can be combined with the carrier materials to produce a single dosage form varies depending upon the host treated and the particular mode of administration. Preferably, a composition of the invention should be formulated so that a dosage of between 0.01-100 mg/kg body weight/day of a compound of the invention can be administered.

In those compositions which comprise one or more other therapeutic agent(s), the one or more other therapeutic agent(s) and a compound of the invention can act synergistically. Therefore, the amount of the one or more other therapeutic agent(s) in such compositions may be less than that required in a monotherapy utilizing only that therapeutic agent. In such compositions a dosage of between 0.01-1,000 μg/kg body weight/day of the one or more other therapeutic agent(s) can be administered.

The amount of one or more other therapeutic agent(s) present in the compositions of this invention may be no more than the amount that would normally be administered in a composition comprising that therapeutic agent as the only active agent. Preferably the amount of one or more other therapeutic agent(s) in the presently disclosed compositions ranges from about 50% to 100% of the amount normally present in a composition comprising that agent as the only therapeutically active agent. In some embodiments, one or more other therapeutic agent(s) is administered at a dosage of about 50%, about 55%, about 60%, about 65%, about 70%, about 75%, about 80%, about 85%, about 90%, or about 95% of the amount normally administered for that agent. As used herein, the phrase “normally administered” means the amount an FDA approved therapeutic agent is provided for dosing per the FDA label insert.

The compounds of this invention, or pharmaceutical compositions thereof, can also be incorporated into compositions for coating an implantable medical device, such as prostheses, artificial valves, vascular grafts, stents and catheters. Vascular stents, for example, have been used to overcome restenosis (re-narrowing of the vessel wall after injury). However, patients using stents or other implantable devices risk clot formation or platelet activation. These unwanted effects may be prevented or mitigated by pre-coating the device with a pharmaceutically acceptable composition comprising a kinase inhibitor. Implantable devices coated with a compound of this invention are another embodiment of the present invention.

In some embodiments, one or more other therapeutic agent is a ATM kinase inhibitor.

In some embodiments, one or more other therapeutic agent is a ATR kinase inhibitor.

In some embodiments, one or more other therapeutic agent is a CHK1 kinase inhibitor.

In some embodiments, one or more other therapeutic agent is a WEE1 inhibitor.

In some embodiments, one or more other therapeutic agent is an immune checkpoint inhibitor. In some embodiments, the immune checkpoint inhibitor is selected from a PD-1 antagonist, a PD-L1 antagonist, and a CTLA-4 antagonist, an OX40 agonist, a CD137 (also called 4-1BB) agonist, a CD27 agonist

### Exemplary Other Therapeutic Agents

In some embodiments, one or more other therapeutic agent is a Poly ADP ribose polymerase (PARP) inhibitor. In some embodiments, a PARP inhibitor is selected from olaparib (LYNPARZA®, AstraZeneca); rucaparib (RUBRACA®, Clovis Oncology); niraparib (ZEJULA®, Tesaro); talazoparib (MDV3800/BMN 673/LT00673, Medivation/Pfizer/Biomarin); veliparib (ABT-888, AbbVie); and BGB-290 (BeiGene, Inc.).

In some embodiments, one or more other therapeutic agent is a histone deacetylase (HDAC) inhibitor. In some embodiments, an HDAC inhibitor is selected from vorinostat (ZOLINZA®, Merck); romidepsin (ISTODAX®, Celgene); panobinostat (FARYDAK®, Novartis); belinostat (BELEODAQ®, Spectrum Pharmaceuticals); entinostat (SNDX-275, Syndax Pharmaceuticals) (NCT00866333); and chidamide (EPIDAZA®, HBI-8000, Chipscreen Biosciences, China).

In some embodiments, one or more other therapeutic agent is a CDK inhibitor, such as a CDK4/CDK6 inhibitor. In some embodiments, a CDK 4/6 inhibitor is selected from palbociclib (IBRANCE®, Pfizer); ribociclib (KISQALI®, Novartis); abemaciclib (Ly2835219, Eli Lilly); and trilaciclib (G1T28, G1 Therapeutics).

In some embodiments, one or more other therapeutic agent is a phosphatidylinositol 3 kinase (PI3K) inhibitor. In some embodiments, a PI3K inhibitor is selected from idelalisib (ZYDELIG®, Gilead), alpelisib (BYL719, Novartis), taselisib (GDC-0032, Genentech/Roche); pictilisib (GDC-0941, Genentech/Roche); copanlisib (BAY806946, Bayer); duvelisib (formerly IPI-145, Infinity Pharmaceuticals); PQR309 (Piqur Therapeutics, Switzerland); and TGR1202 (formerly RP5230, TG Therapeutics).

In some embodiments, one or more other therapeutic agent is a platinum-based therapeutic, also referred to as platins. Platins cause cross-linking of DNA, such that they inhibit DNA repair and/or DNA synthesis, mostly in rapidly reproducing cells, such as cancer cells. In some embodiments, a platinum-based therapeutic is selected from cisplatin (PLATINOL®, Bristol-Myers Squibb); carboplatin (PARAPLATIN®, Bristol-Myers Squibb; also, Teva; Pfizer); oxaliplatin (ELOXITIN® Sanofi-Aventis); nedaplatin (AQUPLA®, Shionogi), picoplatin (Poniard Pharmaceuticals); and satraplatin (JM-216, Agennix).

In some embodiments, one or more other therapeutic agent is a taxane compound, which causes disruption of microtubules, which are essential for cell division. In some embodiments, a taxane compound is selected from paclitaxel (TAXOL®, Bristol-Myers Squibb), docetaxel (TAXOTERE®, Sanofi-Aventis; DOCEFREZ®, Sun Pharmaceutical), albumin-bound paclitaxel (ABRAXANE®; Abraxis/Celgene), cabazitaxel (JEVTANA®, Sanofi-Aventis), and SID530 (SK Chemicals, Co.) (NCT00931008).

In some embodiments, one or more other therapeutic agent is a nucleoside inhibitor, or a therapeutic agent that interferes with normal DNA synthesis, protein synthesis, cell replication, or will otherwise inhibit rapidly proliferating cells.

In some embodiments, a nucleoside inhibitor is selected from trabectedin (guanidine alkylating agent, YONDELIS®, Janssen Oncology), mechlorethamine (alkylating agent, VALCHLOR®, Aktelion Pharmaceuticals); vincristine (ONCOVIN®, Eli Lilly; VINCASAR®, Teva Pharmaceuticals; MARQIBO®, Talon Therapeutics); temozolomide (prodrug to alkylating agent 5-(3-methyltriazen-1-yl)-imidazole-4-carboxamide (MTIC) TEMODAR®, Merck); cytarabine injection (ara-C, antimetabolic cytidine analog, Pfizer); lomustine (alkylating agent, CEENU®, Bristol-Myers Squibb; GLEOSTINE®, NextSource Biotechnology); azacitidine (pyrimidine nucleoside analog of cytidine, VIDAZA®, Celgene); omacetaxine mepesuccinate (cephalotaxine ester) (protein synthesis inhibitor, SYNRIBO®; Teva Pharmaceuticals); asparaginase Erwinia chrysanthemi (enzyme for depletion of asparagine, ELSPAR®, Lundbeck; ERWINAZE®, EUSA Pharma); eribulin mesylate (microtubule inhibitor, tubulin-based antimitotic, HALAVEN®, Eisai); cabazitaxel (microtubule inhibitor, tubulin-based antimitotic, JEVTANA®, Sanofi-Aventis); capacetrine (thymidylate synthase inhibitor, XELODA®, Genentech); bendamustine (bifunctional mechlorethamine derivative, believed to form interstrand DNA cross-links, TREANDA®, Cephalon/Teva); ixabepilone (semi-synthetic analog of epothilone B, microtubule inhibitor, tubulin-based antimitotic, IXEMPRA®, Bristol-Myers Squibb); nelarabine (prodrug of deoxyguanosine analog, nucleoside metabolic inhibitor, ARRANON®, Novartis); clorafabine (prodrug of ribonucleotide reductase inhibitor, competitive inhibitor of deoxycytidine, CLOLAR®, Sanofi-Aventis); and trifluridine and tipiracil (thymidine-based nucleoside analog and thymidine phosphorylase inhibitor, LONSURF®, Taiho Oncology).

In some embodiments, one or more other therapeutic agent is a kinase inhibitor or VEGF-R antagonist. Approved VEGF inhibitors and kinase inhibitors useful in the present invention include: bevacizumab (AVASTIN®, Genentech/Roche) an anti-VEGF monoclonal antibody; ramucirumab (CYRAMZA®, Eli Lilly), an anti-VEGFR-2 antibody and ziv-aflibercept, also known as VEGF Trap (ZALTRAP®; Regeneron/Sanofi). VEGFR inhibitors, such as regorafenib (STIVARGA®, Bayer); vandetanib (CAPRELSA®, AstraZeneca); axitinib (INLYTA®, Pfizer); and lenvatinib (LENVIMA®, Eisai); Raf inhibitors, such as sorafenib (NEXAVAR®, Bayer AG and Onyx); dabrafenib (TAFINLAR®, Novartis); and vemurafenib (ZELBORAF®, Genentech/Roche); MEK inhibitors, such as cobimetanib (COTELLIC®, Exelexis/Genentech/Roche); trametinib (MEKINIST®, Novartis); Bcr-Abl tyrosine kinase inhibitors, such as imatinib (GLEEVEC®, Novartis); nilotinib (TASIGNA®, Novartis); dasatinib (SPRYCEL®, BristolMyersSquibb); bosutinib (BOSULIF®, Pfizer); and ponatinib (INCLUSIG®, Ariad Pharmaceuticals); Her2 and EGFR inhibitors, such as gefitinib (IRESSA®, AstraZeneca); erlotinib (TARCEEVA®, Genentech/Roche/Astellas); lapatinib (TYKERB®, Novartis); afatinib (GILOTRIF®, Boehringer Ingelheim); osimertinib (targeting activated EGFR, TAGRISSO®, AstraZeneca); and brigatinib (ALUNBRIG®, Ariad Pharmaceuticals); c-Met and VEGFR2 inhibitors, such as cabozanitib (COMETRIQ®, Exelexis); and multikinase inhibitors, such as sunitinib (SUTENT®, Pfizer); pazopanib (VOTRIENT®, Novartis); ALK inhibitors, such as crizotinib (XALKORI®, Pfizer); ceritinib (ZYKADIA®, Novartis); and alectinib (ALECENZa®, Genentech/Roche); Bruton's tyrosine kinase inhibitors, such as ibrutinib (IMBRUVICA®, Pharmacyclics/Janssen); and Flt3 receptor inhibitors, such as midostaurin (RYDAPT®, Novartis).

Other kinase inhibitors and VEGF-R antagonists that are in development and may be used in the present invention include tivozanib (Aveo Pharmaecuticals); vatalanib (Bayer/Novartis); lucitanib (Clovis Oncology); dovitinib (TK1258, Novartis); Chiauanib (Chipscreen Biosciences); CEP-11981 (Cephalon); linifanib (Abbott Laboratories); neratinib (HKI-272, Puma Biotechnology); radotinib (SUPECT®, IY5511, Il-Yang Pharmaceuticals, S. Korea); ruxolitinib (JAKAFI®, Incyte Corporation); PTC299 (PTC Therapeutics); CP-547,632 (Pfizer); foretinib (Exelexis, GlaxoSmithKline); quizartinib (Daiichi Sankyo) and motesanib (Amgen/Takeda).

In some embodiments, one or more other therapeutic agent is an mTOR inhibitor, which inhibits cell proliferation, angiogenesis and glucose uptake. In some embodiments, an mTOR inhibitor is everolimus (AFINITOR®, Novartis); temsirolimus (TORISEL®, Pfizer); and sirolimus (RAPAMUNE®, Pfizer).

In some embodiments, one or more other therapeutic agent is a proteasome inhibitor. Approved proteasome inhibitors useful in the present invention include bortezomib (VELCADE®, Takeda); carfilzomib (KYPROLIS®, Amgen); and ixazomib (NINLARO®, Takeda).

In some embodiments, one or more other therapeutic agent is a growth factor antagonist, such as an antagonist of platelet-derived growth factor (PDGF), or epidermal growth factor (EGF) or its receptor (EGFR). Approved PDGF antagonists which may be used in the present invention include olaratumab (LARTRUVO®; Eli Lilly). Approved EGFR antagonists which may be used in the present invention include cetuximab (ERBITUX®, Eli Lilly); necitumumab (PORTRAZZA®, Eli Lilly), panitumumab (VECTIBIX®, Amgen); and osimertinib (targeting activated EGFR, TAGRISSO®, AstraZeneca).

In some embodiments, one or more other therapeutic agent is an aromatase inhibitor. In some embodiments, an aromatase inhibitor is selected from exemestane (AROMASIN®, Pfizer); anastazole (ARIMIDEX®, AstraZeneca) and letrozole (FEMARA®, Novartis).

In some embodiments, one or more other therapeutic agent is an antagonist of the hedgehog pathway. Approved hedgehog pathway inhibitors which may be used in the present invention include sonidegib (ODOMZO®, Sun Pharmaceuticals); and vismodegib (ERIVEDGE®, Genentech), both for treatment of basal cell carcinoma.

In some embodiments, one or more other therapeutic agent is a folic acid inhibitor. Approved folic acid inhibitors useful in the present invention include pemetrexed (ALIMTA®, Eli Lilly).

In some embodiments, one or more other therapeutic agent is a CC chemokine receptor 4 (CCR4) inhibitor. CCR4 inhibitors being studied that may be useful in the present invention include mogamulizumab (POTELIGEO®, Kyowa Hakko Kirin, Japan).

In some embodiments, one or more other therapeutic agent is an isocitrate dehydrogenase (IDH) inhibitor. IDH inhibitors being studied which may be used in the present invention include AG120 (Celgene; NCT02677922); AG221 (Celgene, NCT02677922; NCT02577406); BAY1436032 (Bayer, NCT02746081); IDH305 (Novartis, NCT02987010).

In some embodiments, one or more other therapeutic agent is an arginase inhibitor. Arginase inhibitors being studied which may be used in the present invention include AEB1102 (pegylated recombinant arginase, Aeglea Biotherapeutics), which is being studied in Phase 1 clinical trials for acute myeloid leukemia and myelodysplastic syndrome (NCT02732184) and solid tumors (NCT02561234); and CB-1158 (Calithera Biosciences).

In some embodiments, one or more other therapeutic agent is a glutaminase inhibitor. Glutaminase inhibitors being studied which may be used in the present invention include CB-839 (Calithera Biosciences).

In some embodiments, one or more other therapeutic agent is an antibody that binds to tumor antigens, that is, proteins expressed on the cell surface of tumor cells. Approved antibodies that bind to tumor antigens which may be used in the present invention include rituximab (RITUXAN®, Genentech/BiogenIdec); ofatumumab (anti-CD20, ARZERRA®, GlaxoSmithKline); obinutuzumab (anti-CD20, GAZYVA®, Genentech), ibritumomab (anti-CD20 and Yttrium-90, ZEVALIN®, Spectrum Pharmaceuticals); daratumumab (anti-CD38, DARZALEX®, Janssen Biotech), dinutuximab (anti-glycolipid GD2, UNITUXIN®, United Therapeutics); trastuzumab (anti-HER2, HERCEPTIN®, Genentech); ado-trastuzumab emtansine (anti-HER2, fused to emtansine, KADCYLA®, Genentech); and pertuzumab (anti-HER2, PERJETA®, Genentech); and brentuximab vedotin (anti-CD30-drug conjugate, ADCETRIS®, Seattle Genetics).

In some embodiments, one or more other therapeutic agent is a topoisomerase inhibitor. Approved topoisomerase inhibitors useful in the present invention include irinotecan (ONIVYDE®, Merrimack Pharmaceuticals); topotecan (HYCAMTIN®, GlaxoSmithKline). Topoisomerase inhibitors being studied which may be used in the present invention include pixantrone (PIXUVRI®, CTI Biopharma).

In some embodiments, one or more other therapeutic agent is an inhibitor of anti-apoptotic proteins, such as BCL-2. Approved anti-apoptotics which may be used in the present invention include venetoclax (VENCLEXTA®, AbbVie/Genentech); and blinatumomab (BLINCYTO®, Amgen). Other therapeutic agents targeting apoptotic proteins which have undergone clinical testing and may be used in the present invention include navitoclax (ABT-263, Abbott), a BCL-2 inhibitor (NCT02079740).

In some embodiments, one or more other therapeutic agent is an androgen receptor inhibitor. Approved androgen receptor inhibitors useful in the present invention include enzalutamide (XTANDI®, Astellas/Medivation); approved inhibitors of androgen synthesis include abiraterone (ZYTIGA®, Centocor/Ortho); approved antagonist of gonadotropin-releasing hormone (GnRH) receptor (degaralix, FIRMAGON®, Ferring Pharmaceuticals).

In some embodiments, one or more other therapeutic agent is a selective estrogen receptor modulator (SERM), which interferes with the synthesis or activity of estrogens. Approved SERMs useful in the present invention include raloxifene (EVISTA®, Eli Lilly).

In some embodiments, the selective estrogen receptor modulator (SERM) is lasofoxifene.

In some embodiments, one or more other therapeutic agent is a selective estrogen receptor degrader (SERD). Approved SERDs useful in the present invention include Elacestrant (Orserdu).

In some embodiments, one or more other therapeutic agent is an inhibitor of bone resorption. An approved therapeutic which inhibits bone resorption is Denosumab (XGEVA®, Amgen), an antibody that binds to RANKL, prevents binding to its receptor RANK, found on the surface of osteoclasts, their precursors, and osteoclast-like giant cells, which mediates bone pathology in solid tumors with osseous metastases. Other approved therapeutics that inhibit bone resorption include bisphosphonates, such as zoledronic acid (ZOMETA®, Novartis).

In some embodiments, one or more other therapeutic agent is an inhibitor of interaction between the two primary p53 suppressor proteins, MDMX and MDM2. Inhibitors of p53 suppression proteins being studied which may be used in the present invention include ALRN-6924 (Aileron), a stapled peptide that equipotently binds to and disrupts the interaction of MDMX and MDM2 with p53. ALRN-6924 is currently being evaluated in clinical trials for the treatment of AML, advanced myelodysplastic syndrome (MDS) and peripheral T-cell lymphoma (PTCL) (NCT02909972; NCT02264613).

In some embodiments, one or more other therapeutic agent is an inhibitor of transforming growth factor-beta (TGF-beta or TGFβ). Inhibitors of TGF-beta proteins being studied which may be used in the present invention include NIS793 (Novartis), an anti-TGF-beta antibody being tested in the clinic for treatment of various cancers, including breast, lung, hepatocellular, colorectal, pancreatic, prostate and renal cancer (NCT 02947165). In some embodiments, the inhibitor of TGF-beta proteins is fresolimumab (GC1008; Sanofi-Genzyme), which is being studied for melanoma (NCT00923169); renal cell carcinoma (NCT00356460); and non-small cell lung cancer (NCT02581787). Additionally, in some embodiments, the additional therapeutic agent is a TGF-beta trap, such as described in Connolly et al. (2012) Int'l J. Biological Sciences 8:964-978. One therapeutic compound currently in clinical trials for treatment of solid tumors is M7824 (Merck KgaA—formerly MSB0011459X), which is a bispecific, anti-PD-L1/TGF-β trap compound (NCT02699515); and (NCT02517398). M7824 is comprised of a fully human IgG1 antibody against PD-L1 fused to the extracellular domain of human TGF-beta receptor II, which functions as a TGF-β“trap.”

In some embodiments, one or more other therapeutic agent is selected from glembatumumab vedotin-monomethyl auristatin E (MMAE) (Celldex), an anti-glycoprotein NMB (gpNMB) antibody (CR011) linked to the cytotoxic MMAE. gpNMB is a protein overexpressed by multiple tumor types associated with cancer cells' ability to metastasize.

In some embodiments, one or more other therapeutic agents is an antiproliferative compound. Such antiproliferative compounds include, but are not limited to aromatase inhibitors; antiestrogens; topoisomerase I inhibitors; topoisomerase II inhibitors; microtubule active compounds; alkylating compounds; histone deacetylase inhibitors; compounds which induce cell differentiation processes; cyclooxygenase inhibitors; MMP inhibitors; mTOR inhibitors; antineoplastic antimetabolites; platin compounds; compounds targeting/decreasing a protein or lipid kinase activity and further anti-angiogenic compounds; compounds which target, decrease or inhibit the activity of a protein or lipid phosphatase; gonadorelin agonists; anti-androgens; methionine aminopeptidase inhibitors; matrix metalloproteinase inhibitors; bisphosphonates; biological response modifiers; antiproliferative antibodies; heparanase inhibitors; inhibitors of Ras oncogenic isoforms; telomerase inhibitors; proteasome inhibitors; compounds used in the treatment of hematologic malignancies; compounds which target, decrease or inhibit the activity of Flt-3; Hsp90 inhibitors such as 17-AAG (17-allylaminogeldanamycin, NSC330507), 17-DMAG (17-dimethylaminoethylamino-17-demethoxy-geldanamycin, NSC707545), IPI-504, TEMODAL CNF1010, CNF2024, CNF1010 from Conforma Therapeutics; temozolomide (TEMODAL®); kinesin spindle protein inhibitors, such as SB715992 or SB743921 from GlaxoSmithKline, or pentamidine/chlorpromazine from CombinatoRx; MEK inhibitors such as ARRY142886 from Array BioPharma, AZd₆244 from AstraZeneca, PD181461 from Pfizer and leucovorin.

The term “aromatase inhibitor” as used herein relates to a compound which inhibits estrogen production, for instance, the conversion of the substrates androstenedione and testosterone to estrone and estradiol, respectively. The term includes, but is not limited to steroids, especially atamestane, exemestane and formestane and, in particular, non-steroids, especially aminoglutethimide, roglethimide, pyridoglutethimide, trilostane, testolactone, ketokonazole, vorozole, fadrozole, anastrozole and letrozole. Exemestane is marketed under the trade name AROMASIN™. Formestane is marketed under the trade name LENTARON™. Fadrozole is marketed under the trade name AFEMA™. Anastrozole is marketed under the trade name ARIMIDEX™. Letrozole is marketed under the trade names FEMARA™ or FEMAr™. Aminoglutethimide is marketed under the trade name ORIMETEN™. A combination of the invention comprising a chemotherapeutic agent which is an aromatase inhibitor is particularly useful for the treatment of hormone receptor positive tumors, such as breast tumors.

The term “antiestrogen” as used herein relates to a compound which antagonizes the effect of estrogens at the estrogen receptor level. The term includes, but is not limited to tamoxifen, fulvestrant, raloxifene and raloxifene hydrochloride. Tamoxifen is marketed under the trade name NOLVADEX™ Raloxifene hydrochloride is marketed under the trade name EVISTA™. Fulvestrant can be administered under the trade name FASLODEX™. A combination of the invention comprising a chemotherapeutic agent which is an antiestrogen is particularly useful for the treatment of estrogen receptor positive tumors, such as breast tumors.

The term “anti-androgen” as used herein relates to any substance which is capable of inhibiting the biological effects of androgenic hormones and includes, but is not limited to, bicalutamide (CASODEX™). The term “gonadorelin agonist” as used herein includes, but is not limited to abarelix, goserelin, and goserelin acetate. Goserelin can be administered under the trade name ZOLADEX™

The term “topoisomerase I inhibitor” as used herein includes, but is not limited to topotecan, gimatecan, irinotecan, camptothecian and its analogues, 9-nitrocamptothecin and the macromolecular camptothecin conjugate PNU-166148. Irinotecan can be administered, e.g., in the form as it is marketed, e.g., under the trademark CAMPTOSAR™. Topotecan is marketed under the trade name HYCAMPTIN™

The term “topoisomerase II inhibitor” as used herein includes, but is not limited to the anthracyclines such as doxorubicin (including liposomal formulation, such as CAELYX™), daunorubicin, epirubicin, idarubicin and nemorubicin, the anthraquinones mitoxantrone and losoxantrone, and the podophillotoxines etoposide and teniposide. Etoposide is marketed under the trade name ETOPOPHOS™ Teniposide is marketed under the trade name VM 26-Bristol Doxorubicin is marketed under the trade name ACRIBLASTIN™ or ADRIAMYCIN™. Epirubicin is marketed under the trade name FARMORUBICIN™. Idarubicin is marketed, under the trade name ZAVEDOS™. Mitoxantrone is marketed under the trade name NOVANTRON™.

The term “microtubule active agent” relates to microtubule stabilizing, microtubule destabilizing compounds and microtublin polymerization inhibitors including, but not limited to taxanes, such as paclitaxel and docetaxel; vinca alkaloids, such as vinblastine or vinblastine sulfate, vincristine or vincristine sulfate, and vinorelbine; discodermolides; cochicine and epothilones and derivatives thereof. Paclitaxel is marketed under the trade name TAXOL™. Docetaxel is marketed under the trade name TAXOTERE™. Vinblastine sulfate is marketed under the trade name VINBLASTIN R.P™. Vincristine sulfate is marketed under the trade name FARMISTIN™.

The term “alkylating agent” as used herein includes, but is not limited to, cyclophosphamide, ifosfamide, melphalan or nitrosourea (BCNU or Gliadel). Cyclophosphamide is marketed under the trade name CYCLOSTIN™. Ifosfamide is marketed under the trade name HOLOXAN™.

The term “histone deacetylase inhibitors” or “HDAC inhibitors” relates to compounds which inhibit the histone deacetylase and which possess antiproliferative activity. This includes, but is not limited to, suberoylanilide hydroxamic acid (SAHA).

The term “antineoplastic antimetabolite” includes, but is not limited to, 5-fluorouracil or 5-FU, capecitabine, gemcitabine, DNA demethylating compounds, such as 5-azacytidine and decitabine, methotrexate and edatrexate, and folic acid antagonists such as pemetrexed. Capecitabine is marketed under the trade name XELODA™. Gemcitabine is marketed under the trade name GEMZAR™.

The term “platin compound” as used herein includes, but is not limited to, carboplatin, cis-platin, cisplatinum and oxaliplatin. Carboplatin can be administered, e.g., in the form as it is marketed, e.g., under the trademark CARBOPLAT™. Oxaliplatin can be administered, e.g., in the form as it is marketed, e.g. under the trademark ELOXATIN™.

The term “compounds targeting/decreasing a protein or lipid kinase activity; or a protein or lipid phosphatase activity; or further anti-angiogenic compounds” as used herein includes, but is not limited to, protein tyrosine kinase and/or serine and/or threonine kinase inhibitors or lipid kinase inhibitors, such as a) compounds targeting, decreasing or inhibiting the activity of the platelet-derived growth factor-receptors (PDGFR), such as compounds which target, decrease or inhibit the activity of PDGFR, especially compounds which inhibit the PDGF receptor, such as an N-phenyl-2-pyrimidine-amine derivative, such as imatinib, SU101, SU6668 and GFB-111; b) compounds targeting, decreasing or inhibiting the activity of the fibroblast growth factor-receptors (FGFR); c) compounds targeting, decreasing or inhibiting the activity of the insulin-like growth factor receptor I (IGF-IR), such as compounds which target, decrease or inhibit the activity of IGF-IR, especially compounds which inhibit the kinase activity of IGF-I receptor, or antibodies that target the extracellular domain of IGF-I receptor or its growth factors; d) compounds targeting, decreasing or inhibiting the activity of the Trk receptor tyrosine kinase family, or ephrin B4 inhibitors; e) compounds targeting, decreasing or inhibiting the activity of the AxI receptor tyrosine kinase family; f) compounds targeting, decreasing or inhibiting the activity of the Ret receptor tyrosine kinase; g) compounds targeting, decreasing or inhibiting the activity of the Kit/SCFR receptor tyrosine kinase, such as imatinib; h) compounds targeting, decreasing or inhibiting the activity of the C-kit receptor tyrosine kinases, which are part of the PDGFR family, such as compounds which target, decrease or inhibit the activity of the c-Kit receptor tyrosine kinase family, especially compounds which inhibit the c-Kit receptor, such as imatinib; i) compounds targeting, decreasing or inhibiting the activity of members of the c-Abl family, their gene-fusion products (e.g., BCR-Abl kinase) and mutants, such as compounds which target decrease or inhibit the activity of c-Abl family members and their gene fusion products, such as an N-phenyl-2-pyrimidine-amine derivative, such as imatinib or nilotinib (AMN107); PD180970; AG957; NSC 680410; PD173955 from ParkeDavis; or dasatinib (BMS-354825); j) compounds targeting, decreasing or inhibiting the activity of members of the protein kinase C (PKC) and Raf family of serine/threonine kinases, members of the MEK, SRC, JAK/pan-JAK, FAK, PDK1, PKB/Akt, Ras/MAPK, PI3K, SYK, TYK2, BTK and TEC family, and/or members of the cyclin-dependent kinase family (CDK) including staurosporine derivatives, such as midostaurin; examples of further compounds include UCN-01, safingol, BAY 43-9006, Bryostatin 1, Perifosine; llmofosine; RO 318220 and RO 320432; GO 6976; lsis 3521; LY333531/LY379196; isochinoline compounds; FTIs; PD184352 or QAN697 (a P13K inhibitor) or AT7519 (CDK inhibitor); k) compounds targeting, decreasing or inhibiting the activity of protein-tyrosine kinase inhibitors, such as compounds which target, decrease or inhibit the activity of protein-tyrosine kinase inhibitors include imatinib mesylate (GLEEVEC™) or tyrphostin such as Tyrphostin A23/RG-50810; AG 99; Tyrphostin AG 213; Tyrphostin AG 1748; Tyrphostin AG 490; Tyrphostin B44; Tyrphostin B44 (+) enantiomer; Tyrphostin AG 555; AG 494; Tyrphostin AG 556, AG957 and adaphostin (4-{[(2,5-dihydroxyphenyl)methyl]amino}-benzoic acid adamantyl ester; NSC 680410, adaphostin); 1) compounds targeting, decreasing or inhibiting the activity of the epidermal growth factor family of receptor tyrosine kinases (EGFR₁ ErbB2, ErbB3, ErbB4 as homo- or heterodimers) and their mutants, such as compounds which target, decrease or inhibit the activity of the epidermal growth factor receptor family are especially compounds, proteins or antibodies which inhibit members of the EGF receptor tyrosine kinase family, such as EGF receptor, ErbB2, ErbB3 and ErbB4 or bind to EGF or EGF related ligands, CP 358774, ZD 1839, ZM 105180; trastuzumab (HERCEPTIN™), cetuximab (ERBITUX™), Iressa, Tarceva, OSI-774, C1-1033, EKB-569, GW-2016, ELI, E2.4, E2.5, E6.2, E6.4, E2.11, E6.3 or E7.6.3, and 7H-pyrrolo-[2,3-d]pyrimidine derivatives; m) compounds targeting, decreasing or inhibiting the activity of the c-Met receptor, such as compounds which target, decrease or inhibit the activity of c-Met, especially compounds which inhibit the kinase activity of c-Met receptor, or antibodies that target the extracellular domain of c-Met or bind to HGF, n) compounds targeting, decreasing or inhibiting the kinase activity of one or more JAK family members (JAK1/JAK2/JAK3/TYK2 and/or pan-JAK), including but not limited to PRT-062070, SB-1578, baricitinib, pacritinib, momelotinib, VX-509, AZD-1480, TG-101348, tofacitinib, and ruxolitinib; o) compounds targeting, decreasing or inhibiting the kinase activity of PI3 kinase (PI3K) including but not limited to ATU-027, SF-1126, DS-7423, PBI-05204, GSK-2126458, ZSTK-474, buparlisib, pictrelisib, PF-4691502, BYL-719, dactolisib, XL-147, XL-765, and idelalisib; and; and q) compounds targeting, decreasing or inhibiting the signaling effects of hedgehog protein (Hh) or smoothened receptor (SMO) pathways, including but not limited to cyclopamine, vismodegib, itraconazole, erismodegib, and IPI-926 (saridegib).

The term “PI3K inhibitor” as used herein includes, but is not limited to compounds having inhibitory activity against one or more enzymes in the phosphatidylinositol-3-kinase family, including, but not limited to PI3Kα, PI3Kγ, PI3Kδ, PI3Kβ, PI3K-C2α, PI3K-C2β, PI3K-C2γ, Vps34, p110-α, p110-β, p110-γ, p110-δ, p85-α, p85-β, p55-γ, p150, p101, and p87. Examples of PI3K inhibitors useful in this invention include but are not limited to ATU-027, SF-1126, DS-7423, PBI-05204, GSK-2126458, ZSTK-474, buparlisib, pictrelisib, PF-4691502, BYL-719, dactolisib, XL-147, XL-765, and idelalisib.

The term “Bcl-2 inhibitor” as used herein includes, but is not limited to compounds having inhibitory activity against B-cell lymphoma 2 protein (Bcl-2), including but not limited to ABT-199, ABT-731, ABT-737, apogossypol, Ascenta's pan-Bcl-2 inhibitors, curcumin (and analogs thereof), dual Bcl-2/Bcl-xL inhibitors (Infinity Pharmaceuticals/Novartis Pharmaceuticals), Genasense (G3139), HA14-1 (and analogs thereof; see WO2008118802), navitoclax (and analogs thereof, see U.S. Pat. No. 7,390,799), NH-1 (Shenayng Pharmaceutical University), obatoclax (and analogs thereof, see WO2004106328), S-001 (Gloria Pharmaceuticals), TW series compounds (Univ. of Michigan), and venetoclax. In some embodiments the Bcl-2 inhibitor is a small molecule therapeutic. In some embodiments the Bcl-2 inhibitor is a peptidomimetic.

The term “BTK inhibitor” as used herein includes, but is not limited to compounds having inhibitory activity against Bruton's Tyrosine Kinase (BTK), including, but not limited to AVL-292 and ibrutinib.

The term “SYK inhibitor” as used herein includes, but is not limited to compounds having inhibitory activity against spleen tyrosine kinase (SYK), including but not limited to PRT-062070, R-343, R-333, Excellair, PRT-062607, and fostamatinib.

Further examples of BTK inhibitory compounds, and conditions treatable by such compounds in combination with compounds of this invention can be found in WO2008039218 and WO2011090760, the entirety of which are incorporated herein by reference.

Further examples of SYK inhibitory compounds, and conditions treatable by such compounds in combination with compounds of this invention can be found in WO2003063794, WO2005007623, and WO2006078846, the entirety of which are incorporated herein by reference.

Further examples of PI3K inhibitory compounds, and conditions treatable by such compounds in combination with compounds of this invention can be found in WO2004019973, WO2004089925, WO2007016176, U.S. Pat. No. 8,138,347, WO2002088112, WO2007084786, WO2007129161, WO2006122806, WO2005113554, and WO2007044729 the entirety of which are incorporated herein by reference.

Further examples of JAK inhibitory compounds, and conditions treatable by such compounds in combination with compounds of this invention can be found in WO2009114512, WO2008109943, WO2007053452, WO2000142246, and WO2007070514, the entirety of which are incorporated herein by reference.

Further anti-angiogenic compounds include compounds having another mechanism for their activity, e.g., unrelated to protein or lipid kinase inhibition e.g., thalidomide (THALOMID™) and TNP-470.

Examples of proteasome inhibitors useful for use in combination with compounds of the invention include, but are not limited to bortezomib, disulfiram, epigallocatechin-3-gallate (EGCG), salinosporamide A, carfilzomib, ONX-0912, CEP-18770, and MLN9708.

Compounds which target, decrease or inhibit the activity of a protein or lipid phosphatase are e.g. inhibitors of phosphatase 1, phosphatase 2A, or CDC25, such as okadaic acid or a derivative thereof.

Compounds which induce cell differentiation processes include, but are not limited to, retinoic acid, α- γ- or δ-tocopherol or α- γ- or δ-tocotrienol.

The term cyclooxygenase inhibitor as used herein includes, but is not limited to, Cox-2 inhibitors, 5-alkyl substituted 2-arylaminophenylacetic acid and derivatives, such as celecoxib (CELEBREX™), rofecoxib (VIOXX™), etoricoxib, valdecoxib or a 5-alkyl-2-arylaminophenylacetic acid, such as 5-methyl-2-(2′-chloro-6′-fluoroanilino)phenyl acetic acid, lumiracoxib.

The term “bisphosphonates” as used herein includes, but is not limited to, etridonic, clodronic, tiludronic, pamidronic, alendronic, ibandronic, risedronic and zoledronic acid. Etridonic acid is marketed under the trade name DIDRONEL™. Clodronic acid is marketed under the trade name BONEFOS™ Tiludronic acid is marketed under the trade name Skelid™. Pamidronic acid is marketed under the trade name AREDIA™. Alendronic acid is marketed under the trade name FOSAMAX™. Ibandronic acid is marketed under the trade name BONDRANAT™. Risedronic acid is marketed under the trade name ACTONEL™. Zoledronic acid is marketed under the trade name ZOMETA™. The term “mTOR inhibitors” relates to compounds which inhibit the mammalian target of rapamycin (mTOR) and which possess antiproliferative activity such as sirolimus (RAPAMUNE@), everolimus (CERTICAN™), CCI-779 and ABT578.

The term “heparanase inhibitor” as used herein refers to compounds which target, decrease or inhibit heparin sulfate degradation. The term includes, but is not limited to, PI-88. The term “biological response modifier” as used herein refers to a lymphokine or interferons.

The term “inhibitor of Ras oncogenic isoforms”, such as H-Ras, K-Ras, or N-Ras, as used herein refers to compounds which target, decrease or inhibit the oncogenic activity of Ras; for example, a “farnesyl transferase inhibitor” such as L-744832, DK8G557 or R115777 (ZARNESTRA™). The term “telomerase inhibitor” as used herein refers to compounds which target, decrease or inhibit the activity of telomerase. Compounds which target, decrease or inhibit the activity of telomerase are especially compounds which inhibit the telomerase receptor, such as telomestatin.

The term “methionine aminopeptidase inhibitor” as used herein refers to compounds which target, decrease or inhibit the activity of methionine aminopeptidase. Compounds which target, decrease or inhibit the activity of methionine aminopeptidase include, but are not limited to, bengamide or a derivative thereof.

The term “proteasome inhibitor” as used herein refers to compounds which target, decrease or inhibit the activity of the proteasome. Compounds which target, decrease or inhibit the activity of the proteasome include, but are not limited to, Bortezomib (VELCADE™) and MLN 341.

The term “matrix metalloproteinase inhibitor” or (“MMP” inhibitor) as used herein includes, but is not limited to, collagen peptidomimetic and nonpeptidomimetic inhibitors, tetracycline derivatives, e.g., hydroxamate peptidomimetic inhibitor batimastat and its orally bioavailable analogue marimastat (BB-2516), prinomastat (AG3340), metastat (NSC 683551) BMS-279251, BAY 12-9566, TAA211, MMI270B or AAJ996.

The term “compounds used in the treatment of hematologic malignancies” as used herein includes, but is not limited to, FMS-like tyrosine kinase inhibitors, which are compounds targeting, decreasing or inhibiting the activity of FMS-like tyrosine kinase receptors (Flt-3R); interferon, 1-β-D-arabinofuransylcytosine (ara-c) and bisulfan; and ALK inhibitors, which are compounds which target, decrease or inhibit anaplastic lymphoma kinase.

Compounds which target, decrease or inhibit the activity of FMS-like tyrosine kinase receptors (Flt-3R) are especially compounds, proteins or antibodies which inhibit members of the Flt-3R receptor kinase family, such as PKC412, midostaurin, a staurosporine derivative, SU11248 and MLN518.

The term “HSP90 inhibitors” as used herein includes, but is not limited to, compounds targeting, decreasing or inhibiting the intrinsic ATPase activity of HSP90; degrading, targeting, decreasing or inhibiting the HSP90 client proteins via the ubiquitin proteosome pathway. Compounds targeting, decreasing or inhibiting the intrinsic ATPase activity of HSP90 are especially compounds, proteins or antibodies which inhibit the ATPase activity of HSP90, such as 17-allylamino,17-demethoxygeldanamycin (17AAG), a geldanamycin derivative; other geldanamycin related compounds; radicicol and HDAC inhibitors.

The term “antiproliferative antibodies” as used herein includes, but is not limited to, trastuzumab (HERCEPTIN™), Trastuzumab-DM1, erbitux, bevacizumab (AVASTIN™), rituximab (RITUXAN®), PRO64553 (anti-CD40) and 2C4 Antibody. By antibodies is meant intact monoclonal antibodies, polyclonal antibodies, multispecific antibodies formed from at least 2 intact antibodies, and antibodies fragments so long as they exhibit the desired biological activity.

For the treatment of acute myeloid leukemia (AML), compounds of the current invention can be used in combination with standard leukemia therapies, especially in combination with therapies used for the treatment of AML. In particular, compounds of the current invention can be administered in combination with, for example, farnesyl transferase inhibitors and/or other drugs useful for the treatment of AML, such as Daunorubicin, Adriamycin, Ara-C, VP-16, Teniposide, Mitoxantrone, Idarubicin, Carboplatinum and PKC412.

Other anti-leukemic compounds include, for example, Ara-C, a pyrimidine analog, which is the 2′-alpha-hydroxy ribose (arabinoside) derivative of deoxycytidine. Also included is the purine analog of hypoxanthine, 6-mercaptopurine (6-MP) and fludarabine phosphate. Compounds which target, decrease or inhibit activity of histone deacetylase (HDAC) inhibitors such as sodium butyrate and suberoylanilide hydroxamic acid (SAHA) inhibit the activity of the enzymes known as histone deacetylases. Specific HDAC inhibitors include MS275, SAHA, FK228 (formerly FR901228), Trichostatin A and compounds disclosed in U.S. Pat. No. 6,552,065 including, but not limited to, N-hydroxy-3-[4-[[[2-(2-methyl-1H-indol-3-yl)-ethyl]-amino]methyl]phenyl]-2E-2-propenamide, or a pharmaceutically acceptable salt thereof and N-hydroxy-3-[4-[(2-hydroxyethyl){2-(1H-indol-3-yl)ethyl]-amino]methyl]phenyl]-2E-2-propenamide, or a pharmaceutically acceptable salt thereof, especially the lactate salt. Somatostatin receptor antagonists as used herein refer to compounds which target, treat or inhibit the somatostatin receptor such as octreotide, and SOM230. Tumor cell damaging approaches refer to approaches such as ionizing radiation. The term “ionizing radiation” referred to above and hereinafter means ionizing radiation that occurs as either electromagnetic rays (such as X-rays and gamma rays) or particles (such as alpha and beta particles). Ionizing radiation is provided in, but not limited to, radiation therapy and is known in the art. See Hellman, Principles of Radiation Therapy, Cancer, in Principles and Practice of Oncology, Devita et al., Eds., 4^{th} Edition, Vol. 1, pp. 248-275 (1993).

Also included are EDG binders and ribonucleotide reductase inhibitors. The term “EDG binders” as used herein refers to a class of immunosuppressants that modulates lymphocyte recirculation, such as FTY720. The term “ribonucleotide reductase inhibitors” refers to pyrimidine or purine nucleoside analogs including, but not limited to, fludarabine and/or cytosine arabinoside (ara-C), 6-thioguanine, 5-fluorouracil, cladribine, 6-mercaptopurine (especially in combination with ara-C against ALL) and/or pentostatin. Ribonucleotide reductase inhibitors are especially hydroxyurea or 2-hydroxy-1H-isoindole-1,3-dione derivatives.

Also included are in particular those compounds, proteins or monoclonal antibodies of VEGF such as 1-(4-chloroanilino)-4-(4-pyridylmethyl)phthalazine or a pharmaceutically acceptable salt thereof, 1-(4-chloroanilino)-4-(4-pyridylmethyl)phthalazine succinate; ANGIOSTATIN™; ENDOSTATIN™; anthranilic acid amides; ZD4190; Zd₆474; SU5416; SU6668; bevacizumab; or anti-VEGF antibodies or anti-VEGF receptor antibodies, such as rhuMAb and RHUFab, VEGF aptamer such as Macugon; FLT-4 inhibitors, FLT-3 inhibitors, VEGFR-2 IgG1 antibody, Angiozyme (RPI 4610) and Bevacizumab (AVASTIN™).

Photodynamic therapy as used herein refers to therapy which uses certain chemicals known as photosensitizing compounds to treat or prevent cancers. Examples of photodynamic therapy include treatment with compounds, such as VISUDYNE™ and porfimer sodium.

Angiostatic steroids as used herein refers to compounds which block or inhibit angiogenesis, such as, e.g., anecortave, triamcinolone, hydrocortisone, 11-α-epihydrocotisol, cortexolone, 17α-hydroxyprogesterone, corticosterone, desoxycorticosterone, testosterone, estrone and dexamethasone.

Implants containing corticosteroids refers to compounds, such as fluocinolone and dexamethasone.

Other chemotherapeutic compounds include, but are not limited to, plant alkaloids, hormonal compounds and antagonists; biological response modifiers, preferably lymphokines or interferons; antisense oligonucleotides or oligonucleotide derivatives; shRNA or siRNA; or miscellaneous compounds or compounds with other or unknown mechanism of action.

The structure of the active compounds identified by code numbers, generic or trade names may be taken from the actual edition of the standard compendium “The Merck Index” or from databases, e.g., Patents International (e.g., IMS World Publications).

### Exemplary Immune Checkpoint Inhibitors

In some embodiments, an immuno-oncology agent is an immune checkpoint inhibitor as described herein.

The term “checkpoint inhibitor” as used herein relates to agents useful in preventing cancer cells from avoiding the immune system of the patient. One of the major mechanisms of anti-tumor immunity subversion is known as “T-cell exhaustion,” which results from chronic exposure to antigens that has led to up-regulation of inhibitory receptors. These inhibitory receptors serve as immune checkpoints in order to prevent uncontrolled immune reactions.

PD-1 and co-inhibitory receptors such as cytotoxic T-lymphocyte antigen 4 (CTLA-4, B and T Lymphocyte Attenuator (BTLA; CD272), T cell Immunoglobulin and Mucin domain-3 (Tim-3), Lymphocyte Activation Gene-3 (Lag-3; CD223), and others are often referred to as a checkpoint regulators. They act as molecular “gatekeepers” that allow extracellular information to dictate whether cell cycle progression and other intracellular signaling processes should proceed.

In some embodiments, an immune checkpoint inhibitor is an antibody to PD-1. PD-1 binds to the programmed cell death 1 receptor (PD-1) to prevent the receptor from binding to the inhibitory ligand PDL-1, thus overriding the ability of tumors to suppress the host anti-tumor immune response.

In some embodiments, the checkpoint inhibitor is a biologic therapeutic or a small molecule. In some embodiments, the checkpoint inhibitor is a monoclonal antibody, a humanized antibody, a fully human antibody, a fusion protein or a combination thereof. In some embodiments, the checkpoint inhibitor inhibits a checkpoint protein selected from CTLA-4, PDL1, PDL2, PD1, B7-H3, B7-H4, BTLA, HVEM, TIM3, GAL9, LAG3, VISTA, KIR, 2B4, CD160, CGEN-15049, CHK 1, CHK2, A2aR, B-7 family ligands or a combination thereof. In some embodiments, the checkpoint inhibitor interacts with a ligand of a checkpoint protein selected from CTLA-4, PDL1, PDL2, PD1, B7-H3, B7-H4, BTLA, HVEM, TIM3, GAL9, LAG3, VISTA, KIR, 2B4, CD160, CGEN-15049, CHK 1, CHK2, A2aR, B-7 family ligands or a combination thereof. In some embodiments, the checkpoint inhibitor is an immunostimulatory agent, a T cell growth factor, an interleukin, an antibody, a vaccine or a combination thereof. In some embodiments, the interleukin is IL-7 or IL-15. In some embodiments, the interleukin is glycosylated IL-7. In an additional aspect, the vaccine is a dendritic cell (DC) vaccine.

Checkpoint inhibitors include any agent that blocks or inhibits in a statistically significant manner, the inhibitory pathways of the immune system. Such inhibitors can include small molecule inhibitors or can include antibodies, or antigen binding fragments thereof, that bind to and block or inhibit immune checkpoint receptors or antibodies that bind to and block or inhibit immune checkpoint receptor ligands. Illustrative checkpoint molecules that can be targeted for blocking or inhibition include, but are not limited to, CTLA-4, PDL1, PDL2, PD1, B7-H3, B7-H4, BTLA, HVEM, GAL9, LAG3, TIM3, VISTA, KIR, 2B4 (belongs to the CD2 family of molecules and is expressed on all NK, γδ, and memory CD8⁺ (αβ) T cells), CD160 (also referred to as BY55), CGEN-15049, CHK 1 and CHK2 kinases, A2aR, and various B-7 family ligands. B7 family ligands include, but are not limited to, B7-1, B7-2, B7-DC, B7-H1, B7-H2, B7-H3, B7-H4, B7-H5, B7-H6 and B7-H7. Checkpoint inhibitors include antibodies, or antigen binding fragments thereof, other binding proteins, biologic therapeutics, or small molecules, that bind to and block or inhibit the activity of one or more of CTLA-4, PDL1, PDL2, PD1, BTLA, HVEM, TIM3, GAL9, LAG3, VISTA, KIR, 2B4, CD 160 and CGEN-15049. Illustrative immune checkpoint inhibitors include, but are not limited to, Tremelimumab (CTLA-4 blocking antibody), anti-OX40, PD-L1 monoclonal Antibody (Anti-B7-H1; MEDI4736), MK-3475 (PD-1 blocker), Nivolumab (anti-PD1 antibody), CT-011 (anti-PD1 antibody), BY55 monoclonal antibody, AMP224 (anti-PDL1 antibody), BMS-936559 (anti-PDL1 antibody), MPLDL3280A (anti-PDL1 antibody), MSB0010718C (anti-PDL1 antibody), and ipilimumab (anti-CTLA-4 checkpoint inhibitor). Checkpoint protein ligands include, but are not limited to PD-L1, PD-L2, B7-H3, B7-H4, CD28, CD86 and TIM-3.

In certain embodiments, the immune checkpoint inhibitor is selected from a PD-1 antagonist, a PD-L1 antagonist, and a CTLA-4 antagonist. In some embodiments, the checkpoint inhibitor is selected from the group consisting of nivolumab (OPDIVO®), ipilimumab (YERVOY®), and pembrolizumab (KEYTRUDA®). In some embodiments, the checkpoint inhibitor is selected from nivolumab (anti-PD-1 antibody, OPDIVO®, Bristol-Myers Squibb); pembrolizumab (anti-PD-1 antibody, KEYTRUDA®, Merck); ipilimumab (anti-CTLA-4 antibody, YERVOY®, Bristol-Myers Squibb); durvalumab (anti-PD-L1 antibody, IMFINZI®, AstraZeneca); and atezolizumab (anti-PD-L1 antibody, TECENTRIQ®, Genentech).

In some embodiments, the checkpoint inhibitor is selected from the group consisting of lambrolizumab (MK-3475), nivolumab (BMS-936558), pidilizumab (CT-011), AMP-224, MDX-1105, MED14736, MPDL3280A, BMS-936559, ipilimumab, lirlumab, IPH2101, pembrolizumab (KEYTRUDA®), and tremelimumab.

In some embodiments, an immune checkpoint inhibitor is REGN2810 (Regeneron), an anti-PD-1 antibody tested in patients with basal cell carcinoma (NCT03132636); NSCLC (NCT03088540); cutaneous squamous cell carcinoma (NCT02760498); lymphoma (NCT02651662); and melanoma (NCT03002376); pidilizumab (CureTech), also known as CT-011, an antibody that binds to PD-1, in clinical trials for diffuse large B-cell lymphoma and multiple myeloma; avelumab (BAVENCIO®, Pfizer/Merck KGaA), also known as MSB0010718C), a fully human IgG1 anti-PD-L1 antibody, in clinical trials for non-small cell lung cancer, Merkel cell carcinoma, mesothelioma, solid tumors, renal cancer, ovarian cancer, bladder cancer, head and neck cancer, and gastric cancer; or PDR001 (Novartis), an inhibitory antibody that binds to PD-1, in clinical trials for non-small cell lung cancer, melanoma, triple negative breast cancer and advanced or metastatic solid tumors. Tremelimumab (CP-675,206; Astrazeneca) is a fully human monoclonal antibody against CTLA-4 that has been in studied in clinical trials for a number of indications, including: mesothelioma, colorectal cancer, kidney cancer, breast cancer, lung cancer and non-small cell lung cancer, pancreatic ductal adenocarcinoma, pancreatic cancer, germ cell cancer, squamous cell cancer of the head and neck, hepatocellular carcinoma, prostate cancer, endometrial cancer, metastatic cancer in the liver, liver cancer, large B-cell lymphoma, ovarian cancer, cervical cancer, metastatic anaplastic thyroid cancer, urothelial cancer, fallopian tube cancer, multiple myeloma, bladder cancer, soft tissue sarcoma, and melanoma. AGEN-1884 (Agenus) is an anti-CTLA4 antibody that is being studied in Phase 1 clinical trials for advanced solid tumors (NCT02694822).

In some embodiments, a checkpoint inhibitor is an inhibitor of T-cell immunoglobulin mucin containing protein-3 (TIM-3). TIM-3 inhibitors that may be used in the present invention include TSR-022, LY3321367 and MBG453. TSR-022 (Tesaro) is an anti-TIM-3 antibody which is being studied in solid tumors (NCT02817633). LY3321367 (Eli Lilly) is an anti-TIM-3 antibody which is being studied in solid tumors (NCT03099109). MBG453 (Novartis) is an anti-TIM-3 antibody which is being studied in advanced malignancies (NCT02608268).

In some embodiments, a checkpoint inhibitor is an inhibitor of T cell immunoreceptor with Ig and ITIM domains, or TIGIT, an immune receptor on certain T cells and NK cells. TIGIT inhibitors that may be used in the present invention include BMS-986207 (Bristol-Myers Squibb), an anti-TIGIT monoclonal antibody (NCT02913313); OMP-313M32 (Oncomed); and anti-TIGIT monoclonal antibody (NCT03119428).

In some embodiments, a checkpoint inhibitor is an inhibitor of Lymphocyte Activation Gene-3 (LAG-3). LAG-3 inhibitors that may be used in the present invention include BMS-986016 and REGN3767 and IMP321. BMS-986016 (Bristol-Myers Squibb), an anti-LAG-3 antibody, is being studied in glioblastoma and gliosarcoma (NCT02658981). REGN3767 (Regeneron), is also an anti-LAG-3 antibody, and is being studied in malignancies (NCT03005782). IMP321 (Immutep S.A.) is an LAG-3-Ig fusion protein, being studied in melanoma (NCT02676869); adenocarcinoma (NCT02614833); and metastatic breast cancer (NCT00349934).

Checkpoint inhibitors that can be used in the present invention include OX40 agonists. OX40 agonists that are being studied in clinical trials include PF-04518600/PF-8600 (Pfizer), an agonistic anti-OX40 antibody, in metastatic kidney cancer (NCT03092856) and advanced cancers and neoplasms (NCT02554812; NCT05082566); GSK3174998 (Merck), an agonistic anti-OX40 antibody, in Phase 1 cancer trials (NCT02528357); MEDI0562 (Medimmune/AstraZeneca), an agonistic anti-OX40 antibody, in advanced solid tumors (NCT02318394 and NCT02705482); MED16469, an agonistic anti-OX40 antibody (Medimmune/AstraZeneca), in patients with colorectal cancer (NCT02559024), breast cancer (NCT01862900), head and neck cancer (NCT02274155) and metastatic prostate cancer (NCT01303705); and BMS-986178 (Bristol-Myers Squibb) an agonistic anti-OX40 antibody, in advanced cancers (NCT02737475).

Checkpoint inhibitors that can be used in the present invention include CD137 (also called 4-1BB) agonists. CD 137 agonists that are being studied in clinical trials include utomilumab (PF-05082566, Pfizer) an agonistic anti-CD137 antibody, in diffuse large B-cell lymphoma (NCT02951156) and in advanced cancers and neoplasms (NCT02554812 and NCT05082566); urelumab (BMS-663513, Bristol-Myers Squibb), an agonistic anti-CD137 antibody, in melanoma and skin cancer (NCT02652455) and glioblastoma and gliosarcoma (NCT02658981); and CTX-471 (Compass Therapeutics), an agonistic anti-CD137 antibody in metastatic or locally advanced malignancies (NCT03881488).

Checkpoint inhibitors that can be used in the present invention include CD27 agonists. CD27 agonists that are being studied in clinical trials include varlilumab (CDX-1127, Celldex Therapeutics) an agonistic anti-CD27 antibody, in squamous cell head and neck cancer, ovarian carcinoma, colorectal cancer, renal cell cancer, and glioblastoma (NCT02335918); lymphomas (NCT01460134); and glioma and astrocytoma (NCT02924038).

Checkpoint inhibitors that can be used in the present invention include glucocorticoid-induced tumor necrosis factor receptor (GITR) agonists. GITR agonists that are being studied in clinical trials include TRX518 (Leap Therapeutics), an agonistic anti-GITR antibody, in malignant melanoma and other malignant solid tumors (NCT01239134 and NCT02628574); GWN323 (Novartis), an agonistic anti-GITR antibody, in solid tumors and lymphoma (NCT 02740270); INCAGN01876 (Incyte/Agenus), an agonistic anti-GITR antibody, in advanced cancers (NCT02697591 and NCT03126110); MK-4166 (Merck), an agonistic anti-GITR antibody, in solid tumors (NCT02132754) and MED11873 (Medimmune/AstraZeneca), an agonistic hexameric GITR-ligand molecule with a human IgG1 Fc domain, in advanced solid tumors (NCT02583165).

Checkpoint inhibitors that can be used in the present invention include inducible T-cell co-stimulator (ICOS, also known as CD278) agonists. ICOS agonists that are being studied in clinical trials include MEDI-570 (Medimmune), an agonistic anti-ICOS antibody, in lymphomas (NCT02520791); GSK3359609 (Merck), an agonistic anti-ICOS antibody, in Phase 1 (NCT02723955); JTX-2011 (Jounce Therapeutics), an agonistic anti-ICOS antibody, in Phase 1 (NCT02904226).

Checkpoint inhibitors that can be used in the present invention include killer IgG-like receptor (KIR) inhibitors. KIR inhibitors that are being studied in clinical trials include lirilumab (IPH2102/BMS-986015, Innate Pharma/Bristol-Myers Squibb), an anti-KIR antibody, in leukemias (NCT01687387, NCT02399917, NCT02481297, NCT02599649), multiple myeloma (NCT02252263), and lymphoma (NCT01592370); IPH2101 (1-7F9, Innate Pharma) in myeloma (NCT01222286 and NCT01217203); and IPH4102 (Innate Pharma), an anti-KIR antibody that binds to three domains of the long cytoplasmic tail (KIR3DL2), in lymphoma (NCT02593045).

Checkpoint inhibitors that can be used in the present invention include CD47 inhibitors of interaction between CD47 and signal regulatory protein alpha (SIRPa). CD47/SIRPa inhibitors that are being studied in clinical trials include ALX-148 (Alexo Therapeutics), an antagonistic variant of (SIRPa) that binds to CD47 and prevents CD47/SIRPa-mediated signaling, in phase 1 (NCT03013218); TTI-621 (SIRPa-Fc, Trillium Therapeutics), a soluble recombinant fusion protein created by linking the N-terminal CD47-binding domain of SIRPa with the Fc domain of human IgG1, acts by binding human CD47, and preventing it from delivering its “do not eat” signal to macrophages, is in clinical trials in Phase 1 (NCT02890368 and NCT02663518); CC-90002 (Celgene), an anti-CD47 antibody, in leukemias (NCT02641002); and Hu5F9-G4 (Forty Seven, Inc.), in colorectal neoplasms and solid tumors (NCT02953782), acute myeloid leukemia (NCT02678338) and lymphoma (NCT02953509).

Checkpoint inhibitors that can be used in the present invention include CD73 inhibitors. CD73 inhibitors that are being studied in clinical trials include MEDI9447 (Medimmune), an anti-CD73 antibody, in solid tumors (NCT02503774); and BMS-986179 (Bristol-Myers Squibb), an anti-CD73 antibody, in solid tumors (NCT02754141).

Checkpoint inhibitors that can be used in the present invention include agonists of stimulator of interferon genes protein (STING, also known as transmembrane protein 173, or TMEM173). Agonists of STING that are being studied in clinical trials include MK-1454 (Merck), an agonistic synthetic cyclic dinucleotide, in lymphoma (NCT03010176); and ADU-S100 (MIW815, Aduro Biotech/Novartis), an agonistic synthetic cyclic dinucleotide, in Phase 1 (NCT02675439 and NCT03172936).

Checkpoint inhibitors that can be used in the present invention include CSF1R inhibitors. CSF1R inhibitors that are being studied in clinical trials include pexidartinib (PLX3397, Plexxikon), a CSF 1R small molecule inhibitor, in colorectal cancer, pancreatic cancer, metastatic and advanced cancers (NCT02777710) and melanoma, non-small cell lung cancer, squamous cell head and neck cancer, gastrointestinal stromal tumor (GIST) and ovarian cancer (NCT02452424); and IMC-CS4 (LY3022855, Lilly), an anti-CSF-1R antibody, in pancreatic cancer (NCT03153410), melanoma (NCT03101254), and solid tumors (NCT02718911); and BLZ945 (4-[2((1R,2R)-2-hydroxycyclohexylamino)-benzothiazol-6-yloxyl]-pyridine-2-carboxylic acid methylamide, Novartis), an orally available inhibitor of CSF1R, in advanced solid tumors (NCT02829723).

Checkpoint inhibitors that can be used in the present invention include NKG2A receptor inhibitors. NKG2A receptor inhibitors that are being studied in clinical trials include monalizumab (IPH2201, Innate Pharma), an anti-NKG2A antibody, in head and neck neoplasms (NCT02643550) and chronic lymphocytic leukemia (NCT02557516).

In some embodiments, the immune checkpoint inhibitor is selected from nivolumab, pembrolizumab, ipilimumab, avelumab, durvalumab, atezolizumab, or pidilizumab.

### EXEMPLIFICATION

### General Synthetic Methods

The following examples are intended to illustrate the invention and are not to be construed as being limitations thereon. Temperatures are given in degrees centigrade. If not mentioned otherwise, all evaporations are performed under reduced pressure, preferably between about 15 mm Hg and 100 mm Hg (=20-133 mbar). The structure of final products, intermediates and starting materials is confirmed by standard analytical methods, e.g., microanalysis and spectroscopic characteristics, e.g., MS, IR, NMR. Abbreviations used are those conventional in the art.

All starting materials, building blocks, reagents, acids, bases, dehydrating agents, solvents, and catalysts utilized to synthesis the compounds of the present invention are either commercially available or can be produced by organic synthesis methods known to one of ordinary skill in the art (Houben-Weyl 4th Ed. 1952, Methods of Organic Synthesis, Thieme, Volume 21). Further, the compounds of the present invention can be produced by organic synthesis methods known to one of ordinary skill in the art as shown in the following examples.

All reactions are carried out under nitrogen or argon unless otherwise stated.

Proton NMR (¹H NMR) is conducted in deuterated solvent. In certain compounds disclosed herein, one or more ¹H shifts overlap with residual proteo solvent signals; these signals have not been reported in the experimental provided hereinafter.

As depicted in the Examples below, in certain exemplary embodiments, compounds are prepared according to the following general procedures. It will be appreciated that, although the general methods depict the synthesis of certain compounds of the present invention, the following general methods, and other methods known to one of ordinary skill in the art, can be applied to all compounds and subclasses and species of each of these compounds, as described herein.

### Abbreviations

Ac: acetyl
ACN: acetonitrile
AcOH: acetic acid
BINAP: (2,2′-bis(diphenylphosphino)-1,1′-binaphthyl)
BnBr: Benzyl bromide
Boc: tert-butoxycarbonyl
Boc2O: di-tert-butyl dicarbonate
Cbz: benzyloxycarbonyl
COD: 1,5-Cyclooctadiene
DCE: 1,2-dichloroethane
DCM: dichloromethane
dcpp: 1,3-Bis(dicyclohexylphosphino)propane bis(tetrafluoroborate)
DDQ: 2,3-Dichloro-5,6-dicyano-1,4-benzoquinone
DIAD: 1,3-bis(dicyclohexylphosphino)propane bis(tetrafluoroborate)
DIPEA or DIEA: N,N-diisopropylethylamine
DMAP: 4-dimethylaminopyridine
DMF: N,N-dimethylformamide
DMSO: dimethyl sulfoxide
EA: ethyl acetate
EDC or EDCI: 1-(3-dimethylaminopropyl)-3-ethylcarbodiimide hydrochloride
ee: enantiomeric excess
eq: equivalents
EtI: ethyl iodide
EtOAc: ethyl acetate
EtOH: ethanol
FA: formic acid
h or hrs: hours
HATU: N,N,N′,N′-tetramethyl-O-(7-azabenzotriazol-1-yl)uronium hexafluorophosphate
HBTU: (2-(1H-benzotriazol-1-yl)-1,1,3,3-tetramethyluronium hexafluorophosphate, hexafluorophosphate
benzotriazole tetramethyl uronium)
HMPA: Hexamethylphosphoramide
HOAc: acetic acid
HOAt: 1-Hydroxy-7-azabenzotriazole
HOBt: Hydroxybenzotriazole
HPLC: high performance liquid chromatography
hr, hrs, or h: hour
IPA or i-PrOH: isopropyl alcohol
M: molar
M: molarity
MeCN: acetonitrile
MeOH: methanol
Mhz megahertz
min: minutes
mL: milliliters
mm: milimeters
mM: millimolar
mmol: millimoles
MS: mass spectrometry or mass spectrometer
MS: Molecule sieves
MTBE: Methyl tert-butyl ether
NaOBH(OAc)3: Sodium triacetoxyborohydride
NMR: Nuclear Magnetic Resonance
Pd(dppf)Cl2: [1,1′-Bis(diphenylphosphino)ferrocene]dichloropalladium(II)
PE: petroleum ether
psi: pounds per square inch
R.T. or rt: room temperature
Rf: retention factor
Rt: retention time
sat: saturated
SFC: supercritical fluid chromatography
t-BuONa: Sodium tert-butoxide
TEA: triethylamine
TFA: trifluoracetic acid
TFE: 2,2,2-trifluoroethanol
THF: tetrahydrofuran
TLC: thin layer chromatography
TMSI: trimethylsilyl iodide
μm: micrometers
μmol: micromoles
v/v: volume per volume

### General Methods

Mass spectra were acquired on LC-MS systems using electrospray, chemical and electron impact ionization methods from a range of instruments of the following configurations: SHIMADZU LCMS-2020, Agilent 1200 LC/G1956A MSD and Agilent 1200\G6110A, Agilent 1200 LC & Agilent 6110 MSD Mass Spectrometer [M+H]⁺ refers to protonated molecular ion of the chemical species.

Chiral HPLC spectra were acquired on SFC systems (Agilent1260 & Berger) using Chiralpak AS-S & AD-S, Chiralcel OD-S & OJ-S.

HPLC spectrum were detected at 254 nm and 220 nm, unless indicated otherwise.

NMR spectra were run on Bruker 400 MHz spectrometers using ICON-NMR, under TopSpin program control. Spectra were measured at 298K, unless indicated otherwise, and were referenced relative to the solvent resonance.

TABLE 2
LCMS Methods

| | | | | Mobile | Mobile |
|---|---|---|---|---|---|
| Method | Gradient | Column | | Phase A | Phase B |
| A | 0% B to 60% | Kinetex EVO | | 0.0375% | 0.01875% |
| | B over 0.6 min, | C18 | | TFA in | TFA in |
| | hold at 60% | (2.1 × 30 | mm, | water | ACN (v/v) |
| | B for 0.18 min, | 5 | μm) | (v/v) | |
| | 60% B to 0% B | | | | |
| | over 0.01 min, | | | | |
| | hold 0% B for | | | | |
| | 0.01 min; | | | | |
| | Flow rate: | | | | |
| | 2.0 mL/min | | | | |
| B | 0% B to 60% | HALO C18 | | 0.0375% | 0.01875% |
| | B over 0.5 min, | (3.0 × 30 | mm, | TFA in | TFA in |
| | hold at 60% | 5.0 | μm) | water | ACN (v/v) |
| | B for 0.3 min, | | | (v/v) | |
| | 60% B to 0% B | | | | |
| | over 0.01 min, | | | | |
| | hold at 0% B | | | | |
| | for 0.24 min; | | | | |
| | Flow rate: | | | | |
| | 1.5 mL/min | | | | |
| D | 5% B to 95% B | Kinetex EVO | | 0.0375% | 0.01875% |
| | over 0.6 min, hold | C18 | | TFA in | TFA in |
| | at 95% B for 0.18 | (2.1 × 30 | mm, | water | ACN (v/v) |
| | min, 95% B to 5% | 5 | μm) | (v/v) | |
| | B over 0.01 min, | | | | |
| | hold at 5% B for | | | | |
| | 0.01 min; | | | | |
| | Flow rate: | | | | |
| | 2 mL/min | | | | |
| E | 5% B to 95% | HALO C18 | | 0.0375% | 0.01875% |
| | B over 0.5 min, | (3.0 × 30 | mm, | TFA in | TFA in |
| | hold at 95% | 5.0 | μm) | water | ACN (v/v) |
| | B for 0.3 min, | | | (v/v) | |
| | 95% B to 5% B | | | | |
| | over 0.01 min, | | | | |
| | hold at 5% B | | | | |
| | for 0.24 min; | | | | |
| | Flow rate: | | | | |
| | 1.5 mL/min | | | | |
| G | 5% B to 95% | Kinetex EVO | | 0.025% | ACN |
| | B over 0.8 min, | C18 | | NH₃•H₂O | |
| | hold at 95% | (2.1 × 30 | mm, | in water | |
| | B for 0.4 min, | 5 | μm) | (v/v) | |
| | 95% B to 5% B | | | | |
| | over 0.01 min, | | | | |
| | hold at 5% | | | | |
| | B for 0.34 min; | | | | |
| | Flow rate: | | | | |
| | 1.5 mL/min | | | | |
| J | 5% B to 40% B; | Chiralpak | | CO₂ | 0.05% |
| | Flow rate: | AD-3 | | | DEA |
| | 3 mL/min | (50 × 4.6 | mm, | | in MeOH |
| | | 3 | μm) | | |

### Example 1: Synthetic Methods

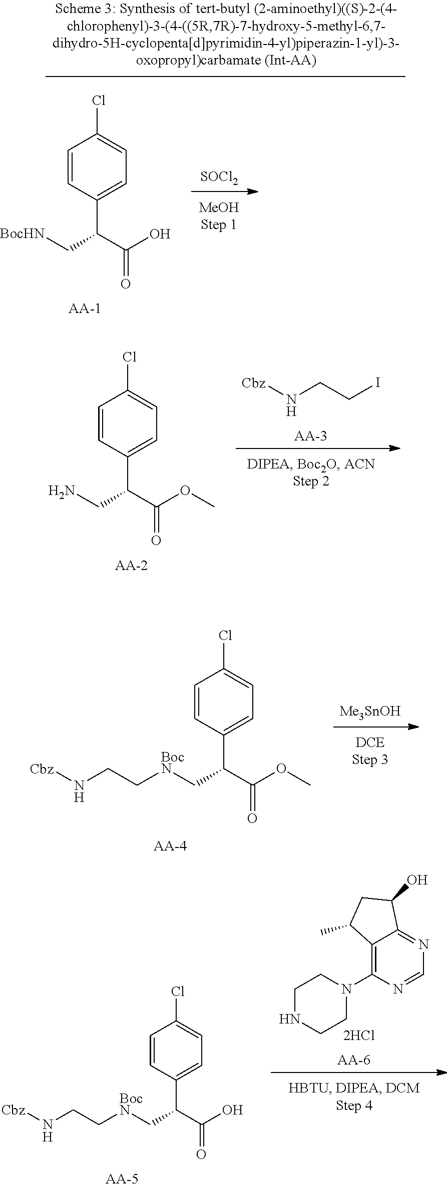

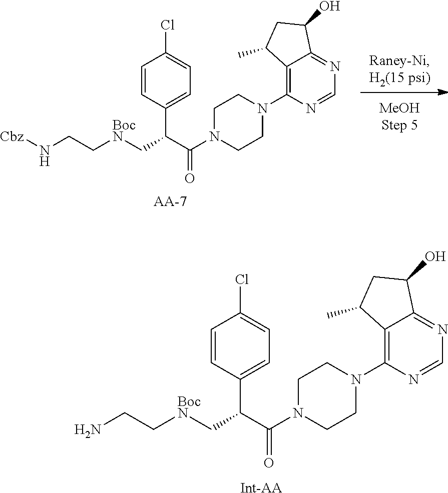

### Step 1: Synthesis of A-2

To a solution of AA-1 (2.15 g, 7.17 mmol, 1 eq) in MeOH (21.5 mL) was added SOCl₂ (1.02 g, 8.61 mmol, 1.2 eq) at 0° C. The reaction mixture was stirred at 25° C. for 12 h. The reaction mixture was concentrated to afford AA-2 (1.8 g, crude, HCl salt) as a white solid. LCMS (Method E): Rt=0.389 min, [M+H]⁺=214.3. SFC: Rt=1.239 min, ee>99%. ¹H NMR (400 MHZ, DMSO-d₆) δ 8.18 (br s, 3H), 7.47 (d, J=8.4 Hz, 2H), 7.35 (d, J=8.4 Hz, 2H), 4.18-4.14 (m, 1H), 3.64 (s, 3H), 3.46-3.43 (m, 1H), 3.13-3.09 (m, 1H).

### Step 2: Synthesis of AA-4

To a solution of AA-2 (1.6 g, 6.40 mmol, 1 eq, HCl salt) in ACN (16 mL) was added AA-3 (2.93 g, 9.60 mmol, 1.5 eq) and DIPEA (2.48 g, 19.19 mmol, 3.34 mL, 3 eq) and the reaction mixture was stirred at 40° C. for 40 h. Then Boc₂O (4.19 g, 19.19 mmol, 4.41 mL, 3 eq) was added and the reaction mixture was stirred at 25° C. for 2 h. The reaction mixture was poured into water (50 mL) and extracted by EA (50 mL*2). The combined organic layers were washed with brine (50 mL), dried over anhydrous sodium sulfate, filtered and concentrated to give residue. The residue was purified by flash silica gel chromatography (40 g silica flash column; eluent: 0-40% ethyl acetate/petroleum ether; gradient at 50 mL/min) to afford AA-4 (1.8 g, 3.59 mmol, 56.17% yield, 98% purity) as colorless oil. LCMS (Method E): Rt=0.637 min, [M+Na]⁺=513.3. SFC: Rt=1.245 min, 1.391 min, ee=93.126%. ¹H NMR (400 MHZ, CDCl₃) δ 7.39-7.28 (m, 7H), 7.22 (br s, 2H), 5.34-4.79 (m, 3H), 4.11-3.86 (m, 1H), 3.82-3.61 (m, 4H), 3.59-3.41 (m, 1H), 3.39-3.05 (m, 4H), 1.44 (s, 9H).

### Step 3: Synthesis of A-5

To a solution of AA-4 (1.8 g, 3.67 mmol, 1 eq) in DCE (18 mL) was added hydroxy (trimethyl) stannane (1.33 g, 7.33 mmol, 2 eq). The reaction mixture was stirred at 80° C. for 36 h. The reaction mixture was diluted with DCM (2 mL). The residue was purified by flash silica gel chromatography (40 g silica flash column; eluent: 0-40% ethyl acetate/petroleum ether; gradient at 50 mL/min) to afford AA-5 (1.5 g, 2.92 mmol, 79.78% yield, 93% purity) as a colorless oil. LCMS (Method E): Rt=0.596 min, [M+Na]⁺=499.3. SFC: Rt=2.271 min, 2.409 min, ee=92%. ¹H NMR (400 MHZ, CDCl₃) δ 7.37-7.28 (m, 7H), 7.24 (d, J=7.2 Hz, 2H), 5.28-4.85 (m, 3H), 4.17-3.91 (m, 1H), 3.82-3.63 (m, 1H), 3.61-3.45 (m, 1H), 3.38-3.05 (m, 4H), 1.44 (s, 9H).

### Step 4: Synthesis of AA-7

To a solution of AA-5 (1.5 g, 3.14 mmol, 1.05 eq) and AA-6 (920.19 mg, 3.00 mmol, 1 eq, 2HCl) in DCM (15 mL) was added HBTU (1.48 g, 3.89 mmol, 1.3 eq) and DIPEA (1.94 g, 14.98 mmol, 2.61 mL, 5 eq). The reaction was stirred at 25° C. for 1 h. The reaction mixture was diluted with DCM (2 mL). The mixture was purified by flash silica gel chromatography (40 g silica flash column; eluent: 0-10% (0.3% NH₃·H₂O in MeOH)/EA at 50 mL/min) to afford AA-7 (1.7 g, 2.45 mmol, 81.87% yield, 100% purity) as a white gum. LCMS (Method E): Rt=0.517 min, [M+H]⁺=693.5. SFC: Rt=0.880 min, 1.605 min, ee-91.9%. ¹H NMR (400 MHZ, CDCl₃) δ 8.50 (s, 1H), 7.40-7.28 (m, 7H), 7.26-7.16 (m, 2H), 5.15-5.05 (m, 3H), 3.90-3.56 (m, 7H), 3.44 (d, J=3.6 Hz, 5H), 3.34-3.14 (m, 5H), 2.20-2.13 (m, 2H), 1.54-1.46 (m, 9H), 1.18-1.10 (m, 3H).

### Step 5: Synthesis of Int-AA

To a slurry of Raney-Ni (900.00 mg, 10.50 mmol, 24.27 eq) in MeOH (3 mL) was added A-7 (300 mg, 432.76 μmol, 1 eq). The reaction mixture was stirred at 25° C. for 48 h under H₂ (15 psi) atmosphere. The reaction mixture was filtered and concentrated to afford Int-AA (950 mg, 1.61 mmol, 93.25% yield, 95% purity) as a white solid. LCMS (Method E): Rt=0.395 min, [M+H]⁺=559.1. SFC: Rt=1.895 min, 2.304 min, ee=82%. ¹H NMR (400 MHZ, DMSO-d₆) δ 8.43 (s, 1H), 7.46-7.42 (m, 2H), 7.30 (d, J=8.4 Hz, 2H), 4.85-4.81 (m, 1H), 4.29-4.27 (m, 1H), 3.70-3.52 (m, 6H), 3.50-3.33 (m, 5H), 3.27-3.18 (m, 2H), 3.10-2.89 (m, 1H), 2.85-2.65 (m, 2H), 2.03-1.84 (m, 2H), 1.41-1.24 (m, 9H), 1.04-0.99 (m, 5H).

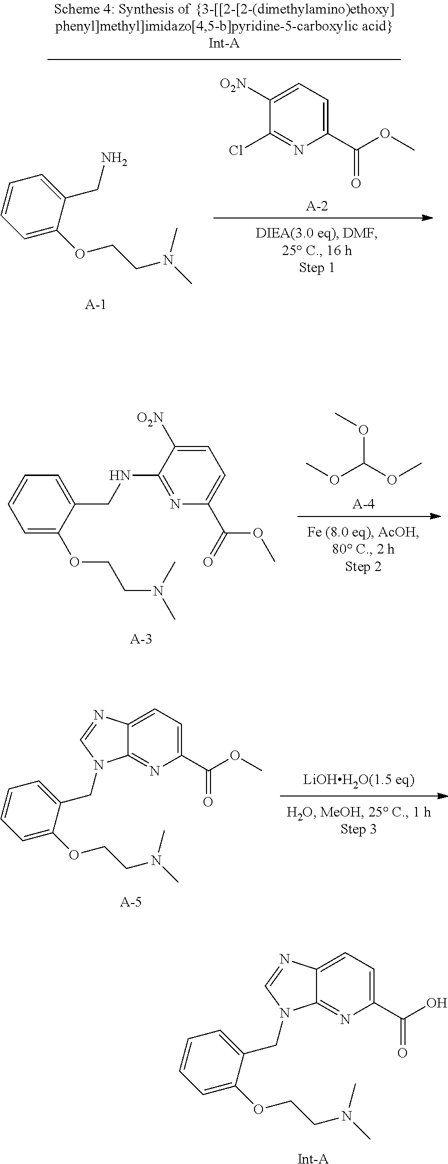

### Step 1: Synthesis of A-3

To A-1 (17 g, 87.51 mmol, 1 eq) was added DIEA (33.93 g, 262.52 mmol, 45.73 mL, 3 eq) in DMF (170 mL). Then A-2 (20.85 g, 96.26 mmol, 1.1 eq) was added. The reaction mixture was stirred at 25° C. for 16 hr. The mixture was quenched with water (500 mL) and aq. HCl (1 N) was added to adjust the pH to 2-3, and the mixture was extracted with EA (100 mL*2). To the aqueous phase was added NaHCO₃ to adjust the pH to 8-9, and it was then extracted with EA (100 mL*3), and the combined organic layers were dried over Na₂SO₄ and concentrated under reduced pressure to give A-3 (32 g, 81.20 mmol, 92.79% yield, 95% purity) as yellow solid. LCMS (Method E): Rt=0.434 min, [M+H]⁺=375.1. ¹H NMR (400 MHZ, CDCl₃) δ 8.79-8.67 (m, 1H), 8.52 (d, J=8.4 Hz, 1H), 7.53 (d, J=6.8 Hz, 1H), 7.37 (d, J=8.4 Hz, 1H), 7.35-7.30 (m, 1H), 7.07-6.93 (m, 2H), 4.97 (br d, J=4.8 Hz, 2H), 4.69-4.56 (m, 2H), 4.03 (s, 3H), 3.66-3.48 (m, 2H), 3.00 (d, J=4.8 Hz, 6H).

### Step 2: Synthesis of A-5

To a solution of A-3 (32 g, 85.47 mmol, 1 eq) and A-4 (72.56 g, 683.78 mmol, 74.96 mL, 8 eq) in AcOH (300 mL) was added iron powder (38.19 g, 683.78 mmol, 8 eq). The mixture was stirred at 80° C. for 2 hr. The mixture was filtered through a pad of celite and the filtrate was adjusted with aq. Na₂CO₃ to pH=10, and then the mixture was extracted with DCM (100 mL*3). The combined organic layers were dried over Na₂SO₄, filtered and concentrated under reduced pressure to give A-5 (30 g, 77.03 mmol, 90.12% yield, 91% purity) as yellow solid. LCMS (Method E): Rt=0.392 min, [M+H]⁺=355.3. ¹H NMR (400 MHZ, DMSO-d₆) δ 8.74 (s, 1H), 8.22 (d, J=8.4 Hz, 1H), 8.02 (d, J=8.4 Hz, 1H), 7.33-7.25 (m, 1H), 7.15-7.09 (m, 1H), 7.05 (d, J=8.4 Hz, 1H), 6.89 (m, 1H), 5.48 (s, 2H), 4.08 (m, 2H), 3.91 (s, 3H), 2.59 (m, 2H), 2.20 (s, 6H).

### Step 3: Synthesis of Int-A

To a solution of A-5 (30 g, 84.65 mmol, 1 eq) in MeOH (200 mL) and H₂O (50 mL) was added LiOH·H₂O (5.33 g, 126.97 mmol, 1.5 eq). The mixture was stirred at 25° C. for 1 hr. The mixture was concentrated in vacuum. The crude product was purified by reversed-phase HPLC. After purification, the eluent was concentrated to remove organic solvents and then lyophilized to give Int-A (29 g, 78.38 mmol, 92.60% yield, 92% purity) as a yellow solid. LCMS (Method E): Rt=0.348 min, [M+H]⁺=341.1. ¹H NMR (400 MHZ, DMSO-d₆) δ 8.61 (s, 1H), 8.12 (d, J=8.4 Hz, 1H), 7.98 (d, J=8.4 Hz, 1H), 7.30-7.23 (m, 1H), 7.09-7.02 (m, 2H), 6.90-6.83 (m, 1H), 5.47 (s, 2H), 4.16 (m, 2H), 2.79 (m, 2H), 2.34 (s, 6H).

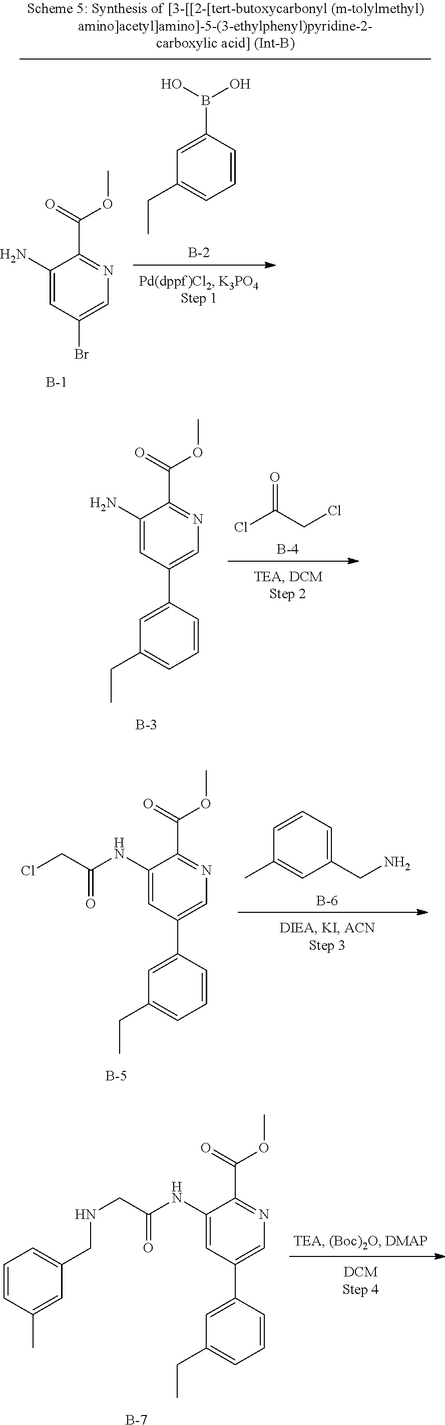

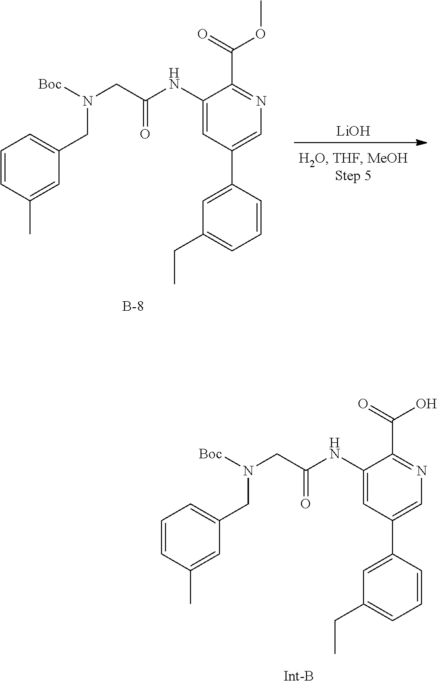

### Step 1: Synthesis of B-3

To a solution of B-1 (5 g, 21.64 mmol, 1 eq) and B-2 (3.89 g, 25.97 mmol, 1.2 eq) in dioxane (50 mL) and H₂O (10 mL) was added Pd(dppf)Cl₂ (1.41 g, 2.16 mmol, 0.1 eq) and K₃PO₄ (13.78 g, 64.92 mmol, 3 eq). The solution was degassed and purged with nitrogen 3 times. The mixture was stirred at 80° C. for 2 hr under nitrogen atmosphere. TLC (PE/EA=1:1. Rf=0.6) showed a new spot. The mixture was poured into water (60 mL) and extracted with EA (60 mL*3), the combined organic layers were dried over Na₂SO₄, filtered and concentrated under reduced pressure to give a residue. The residue was purified by column chromatography (SiO₂, petroleum ether/ethyl acetate=100/1 to 1/100) and concentrated under reduced pressure to give B-3 (4.3 g, 13.81 mmol, 63.84% yield, 82.34% purity) as a yellow solid. LCMS (Method D): Rt: 0.325 min, [M+H]⁺=257.0.

### Step 2: Synthesis of B-5

To a solution of B-3 (3.7 g, 14.44 mmol, 1 eq) in DCM (10 mL) was added TEA (5.84 g, 57.75 mmol, 8.04 mL, 4 eq) and B-4 (4.89 g, 43.31 mmol, 3.45 mL, 3 eq). The mixture was stirred at 0° C. for 1 hr. TLC (PE/EA=1:1. Rf=0.8) showed a new spot. The mixture was poured into water (18 mL) and extracted with DCM (18 mL+3). The combined organic layers were dried over Na₂SO₄, filtered and concentrated under reduced pressure to give a residue. The residue was purified by column chromatography (SiO₂, petroleum ether/ethyl acetate=100/1 to 1/100) and concentrated under reduced pressure to give B-5 (3.3 g, 9.60 mmol, 66.47% yield, 96.77% purity) as a white solid. LCMS (Method D): Rt: 0.429 min, [M+H]⁺=333.0.

### Step 3: Synthesis of B-7

To a solution of B-5 (3.8 g, 11.42 mmol, 1 eq) and B-6 (2.70 g, 17.13 mmol, 2.80 mL, 1.5 eq, HCl salt) in ACN (38 mL) was added DIEA (5.90 g, 45.68 mmol, 7.96 mL, 4 eq) and KI (7.58 g. 45.68 mmol, 4 eq). The mixture was stirred at 40° C. for 1 hr. The mixture was poured into water (40 mL), extracted with DCM (40 mL*3), and the combined organic layers were dried over Na₂SO₄, filtered and concentrated under reduced pressure to give B-7 (5 g, crude) as a yellow solid which was used for the next step without further purification. LCMS (Method D): Rt=0.341 min, [M+H]⁺=418.3.

### Step 4: Synthesis of B-8

To a solution of B-7 (5 g, 11.98 mmol, 1 eq) in DCM (50 mL) was added TEA (1.21 g, 11.98 mmol, 1.67 mL, 1 eq), DMAP (146.31 mg, 1.20 mmol, 0.1 eq) and Boc₂O (5.23 g, 23.95 mmol, 5.50 mL, 2 eq). The mixture was stirred at 25° C. for 1 hr. TLC (PE/EA=1:1, Rf=0.8) showed a new spot. The mixture was poured into water (50 mL), extracted with DCM (50 mL*3), and the combined organic layers were dried over Na₂SO₄, filtered, and concentrated under reduced pressure to give a residue. The residue was purified by column chromatography (SiO₂, petroleum ether/ethyl acetate=100/1 to 1/100) and concentrated under reduced pressure to give B-8 (4.4 g. 8.50 mmol, 70.98% yield, 99% purity) as a yellow solid. LCMS (Method D): Rt=0.570 min, [M+H]⁺=518.2.

### Step 5: Synthesis of Int-B

To a solution of B-8 (4.4 g, 8.50 mmol, 1 eq) in THF (15 mL), H₂O (15 mL) and MeOH (15 mL) was added LiOH·H₂O (1.07 g, 25.50 mmol, 3 eq) and the mixture was stirred at 25° C. for 0.5 hr. The mixture was poured into water (40 mL) and extracted with DCM (40 mL*2), then the pH of the aqueous phase was adjusted to 4-5 with 1 N HCl, extracted with DCM (20 mL*3), and then the combined organic layers were dried over Na₂SO₄, filtered and concentrated under reduced pressure to give Int-B (3.4 g, 6.75 mmol, 79.43% yield, 99% purity) as a yellow solid. LCMS (Method D): Rt=0.479 min, [M+H]⁺=504.2. ¹H NMR (400 MHZ, DMSO-d₆) δ 14.31 (s, 1H), 9.40 (d, J=2.0 Hz, 1H), 8.62 (d, J=1.6 Hz, 1H), 7.68-7.59 (m, 3H), 7.48 (d, J=7.2 Hz, 1H), 7.43-7.37 (m, 1H), 7.34-7.23 (m, 3H), 4.73 (s, 2H), 4.14 (s, 2H), 3.30 (s, 2H), 2.93-2.86 (m, 2H), 2.48 (s, 3H), 1.58 (s, 9H), 1.43 (t, J=7.6 Hz, 3H).

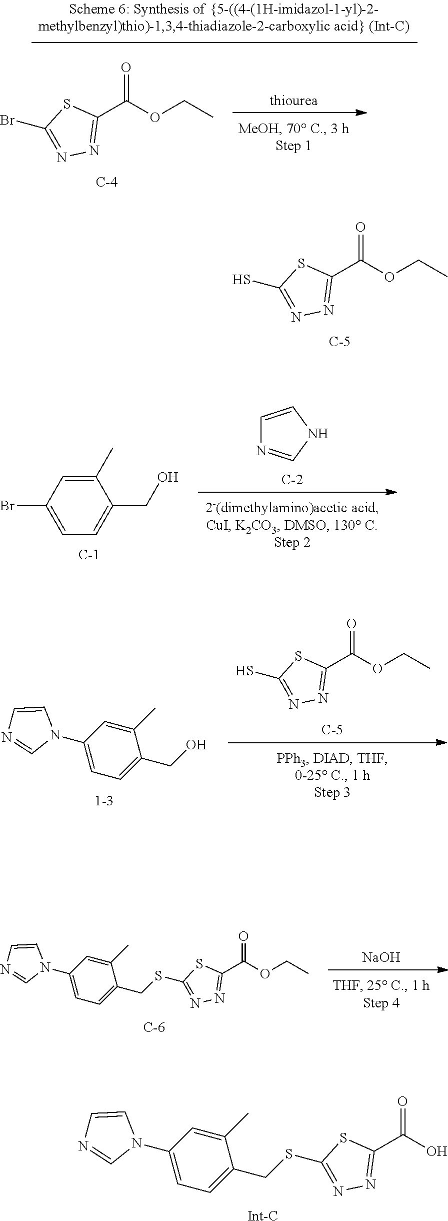

### Step 1: Synthesis of C-5

To a solution of C-4 (40 g, 168.72 mmol, 1 eq) in MeOH (160 mL) was added thiourea (19.27 g, 253.09 mmol, 1.5 eq) under N₂ and the reaction mixture was purged with N₂ three times then stirred at 70° C. for 6 hr. The reaction mixture was concentrated to remove MeOH, then acidified with 1 N aq. HCl (50 mL) at 0° C., and extracted with EA (50 mL*3). The combined organic phase was dried with anhydrous Na₂SO₄ and filtered. The filtrate was concentrated to give C-5 (32 g, 168.21 mmol, 99.69% yield) as a yellow solid which was used in the next step without any further purification. LCMS (Method E): Rt=0.445 min, [M+H]⁺⁺=191.11H NMR (400 MHZ, DMSO-d₆) δ 4.34 (q, J=7.2 Hz, 2H), 1.29 (t, J=7.2 Hz, 3H).

### Step 2: Synthesis of C-3

To a solution of C-1 (50 g, 248.68 mmol, 1 eq) and C-2 (33.86 g, 497.36 mmol, 2 eq) in DMSO (500 mL) was added 2-(dimethylamino) acetic acid (2.56 g, 24.87 mmol, 0.1 eq), K₂CO₃ (34.37 g, 248.68 mmol, 1 eq), and CuI (9.47 g, 49.74 mmol, 0.2 eq) under N₂ and the reaction mixture was purged with N₂ three times and then stirred at 130° C. for 16 hr. The mixture was poured into saturated aq. NaCl (2000 mL) and then filtered and the filter cake was washed with EA (50 mL*3), and then the filtrate extracted with EA (500 mL*8). The combined organic layer was dried with anhydrous Na₂SO₄, filtered and evaporated to afford the crude product. The crude product was triturated with EA (30 mL), filtered and the filter cake was washed with EA (5 mL*3) to give C-3 (31 g, 164.70 mmol, 66.23% yield) as an off-white solid. LCMS (Method E): Rt=0.284 min, [M+H]⁺⁺=189.2. ¹H NMR (400 MHZ, DMSO-d₆) δ 8.22 (br s, 1H), 7.71 (br s, 1H), 7.48-7.40 (m, 3H), 7.09 (br s, 1H), 5.21-5.13 (m, 1H), 4.52 (d, J=5.4 Hz, 2H), 2.30 (s, 3H).

### Step 3: Synthesis of C-6

To a solution of C-3 (21 g, 111.57 mmol, 1 eq) and C-5 (21.23 g, 111.57 mmol, 1 eq) in THF (200 mL) was added PPh₃ (35.12 g, 133.88 mmol, 1.2 eq) at 0° C. under N₂, and then DIAD (27.07 g, 133.88 mmol, 25.96 mL, 1.2 eq) in THF (60 mL) was added dropwise to the mixture at 0-10° C., and then stirred at 0-25° C. for 50 min under N₂. The reaction mixture was quenched with water (1000 mL) and extracted with EA (100 mL*3). The combined organic phase was dried with anhydrous Na₂SO₄, filtered and concentrated to give the crude product. The crude product was triturated with DCM (30 mL), filtered and the filter cake was washed with DCM (8 mL*3). The filtrate was purified by flash silica gel chromatography (330 g*2 SepaFlash silica flash column; eluent: 0-100% PE/EA) and the eluent was concentrated to give C-6 (36 g, 99.87 mmol, 89.52% yield) as a yellow solid. LCMS: Rt=0.561 min, [M+H]⁺⁺=360.9. ¹H NMR (400 MHZ, DMSO-d₆) δ 8.25 (br s, 1H), 7.74 (s, 1H), 7.63-7.52 (m, 2H), 7.46-7.45 (m, 1H), 7.10 (br s, 1H), 4.72 (s, 2H), 4.40 (q, J=7.2 Hz, 2H), 2.46 (s, 3H), 1.33 (t, J=7.2 Hz, 3H).

### Step 4: Synthesis of Int-C

Detailed Synthetic Procedure: To a solution of C-6 (30 g, 83.23 mmol, 1 eq) in THF (300 mL) was added NaOH (1 M, 91.55 mL, 1.1 eq) slowly, the reaction mixture was stirred at 25° C. for 1 hr. White solid was precipitation. The reaction mixture was filtered and the filter cake was washed by THF (5 mL*3), the filter cake was dried under vacuum to give Int-C (23 g, 64.72 mmol, 77.76% yield, Na salt) as white solid. LCMS: Rt: 0.389 min, [M+H]⁺=332.9. ¹H NMR (400 MHz, DMSO-d₆) δ=8.23 (s, 1H), 7.72 (s, 1H), 7.58-7.49 (m, 2H), 7.43-7.40 (m, 1H), 7.08 (s, 1H), 4.58 (s, 2H), 2.44 (s, 3H).

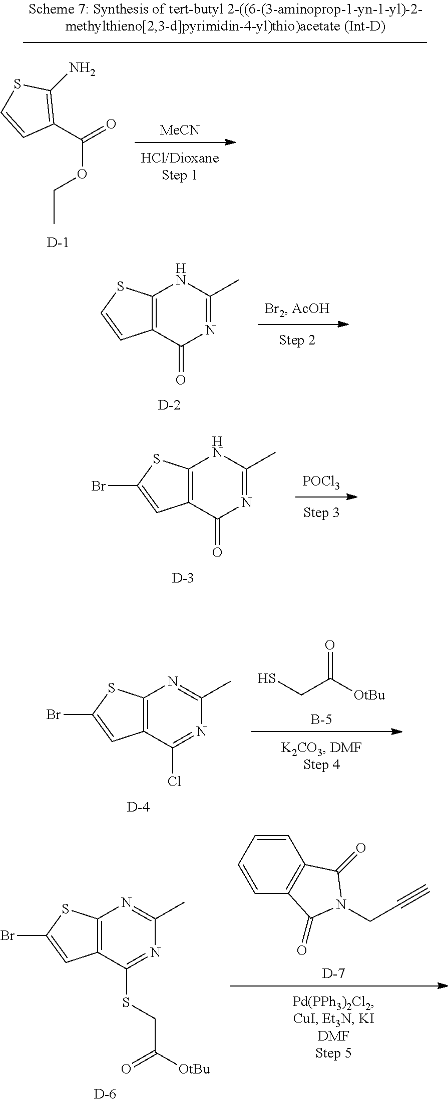

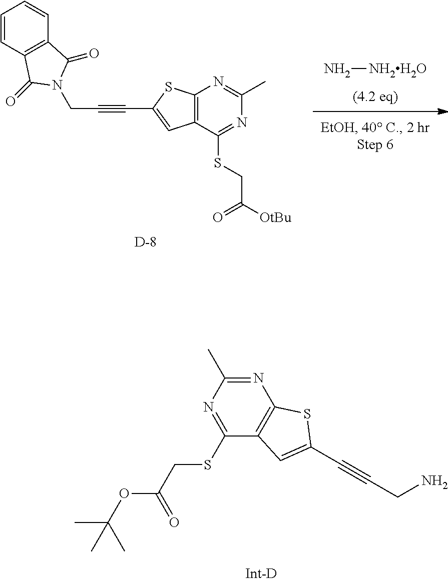

### Step 1: Synthesis of D-2

A mixture of D-1 (54 g, 315.39 mmol, 1 eq), MeCN (19.42 g, 473.09 mmol, 24.90 mL, 1.5 eq) and HCl/dioxane (4 M, 540.00 mL, 6.85 eq) was stirred at 20° C. for 30 mins. Then the mixture was stirred at 50° C. for 2 hrs and at 110° C. for 36 hrs. The reaction mixture was concentrated. The residue was triturated with MeCN (200 mL) at 20° C. 3 times to give D-2 (47 g, 282.79 mmol, 89.66% yield) as a yellow solid.

### Step 2: Synthesis of D-3

To a solution of D-2 (158 g, 950.66 mmol, 1 eq) in AcOH (1200 mL) was added Br₂ (182.31 g, 1.14 mol, 58.81 mL, 1.2 eq) dropwise at 20° C. The mixture was stirred at 20° C. for 16 hrs. The reaction mixture was concentrated to give a residue. The residue was diluted with H₂O (800 mL) and stirred for 30 mins. The mixture was filtered. The filter cake was washed with H₂O (800 mL×2) and dried under vacuum to give D-3 (77 g, crude) as a brown solid. LCMS (Method E): Rt=0.675 min, [M+H]⁺=244.9.

### Step 3: Synthesis of D-4

A suspension of D-3 (77 g, 314.16 mmol, 1 eq) in POCl₃ (495.00 g, 3.23 mol, 300 mL, 10.28 eq) was stirred at 105° C. for 2 hrs. The reaction mixture was concentrated under vacuum to remove the POCl₃. The residue was poured into H₂O (300 mL) at 25° C. The mixture was neutralized with saturated aq. NaHCO₃ and extracted with EA (300 mL×3). The combined organic layer was washed with brine (300 mL), dried over Na₂SO₄, filtered and concentrated to give the crude product. The residue was purified by column chromatography (SiO₂, petroleum ether/ethyl acetate=20/1 to 10/1, PE/EA=5/1, Rf=0.7) and concentrated to give D-4 (35 g, 132.81 mmol, 42.27% yield, 100% purity) as a white solid. LCMS (Method E): Rt=0.887 min, [M+H]⁺=265.0. ¹H NMR (400 MHZ, CDCl₃) δ 7.43 (s, 1H), 2.78 (s, 3H).

### Step 4: Synthesis of D-6

A mixture of D-4 (28 g, 106.24 mmol, 1 eq), D-5 (16.54 g, 111.56 mmol, 1.05 eq) and K₂CO₃ (44.05 g, 318.73 mmol, 3 eq) in DMF (300 mL) was stirred at 60° C. for 16 hrs. The reaction mixture was diluted with H₂O (200 mL) and extracted with EA (200 mL). The organic layer was washed with brine (200 mL×2), dried over Na₂SO₄, filtered and concentrated. The residue was purified by flash silica gel chromatography (30% EA in PE, PE/EA=5/1, Rf=0.4) to give D-6 (35 g, 93.26 mmol, 87.78% yield) as an off-white solid. LCMS (Method E): Rt=1.028 min, [M+H]⁺=375.1. ¹H NMR (400 MHZ, CDCl₃) δ 7.32 (s, 1H), 4.01 (s, 2H), 2.69 (s, 3H), 1.48 (s, 9H).

### Step 5: Synthesis of D-8

To a solution of D-6 (1.0 g. 2.66 mmol, 1 eq), KI (1.33 g, 7.99 mmol, 3 eq) and TEA (1.35 g, 13.32 mmol, 1.85 mL, 5 eq) in DMF (10 mL) was added D-7 (740.11 mg, 4.00 mmol, 1.5 eq), Pd(PPh₃)₂Cl₂ (187.02 mg, 266.45 μmol, 0.1 eq) and CuI (50.75 mg, 266.45 μmol, 0.1 eq). After the addition, the reaction mixture was stirred at 80° C. for 2 hrs under N₂. The reaction mixture was cooled to 20° C., diluted with H₂O (50 mL) and extracted with EA (100 mL×2). The combined organic layer was washed with brine (100 mL), dried over Na₂SO₄, filtered and concentrated. The residue was purified by flash silica gel chromatography (40% EA in PE) to give D-8 (7 g, 14.60 mmol, 54.78% yield, 10 batches in parallel) as a yellow solid. LCMS (Method E): Rt=0.746 min, [M+H]⁺=480.1. ¹H NMR (400 MHZ, CDCl₃) δ 7.92 (dd, J=3.2, 5.6 Hz, 2H), 7.76 (dd, J=3.2, 5.6 Hz, 2H), 7.38 (s, 1H), 4.73 (s, 2H), 3.99 (s, 2H), 2.70 (s, 3H), 1.47 (s, 9H).

### Step 6: Synthesis of Int-D

To a solution of D-8 (200 mg, 417.04 μmol, 1 eq) in EtOH (5 mL) was added hydrazine hydrate (87.68 mg, 1.75 mmol, 84.96 μL, 4.2 eq) at 25° C. The mixture was stirred at 40° C. for 2 hrs. The mixture was diluted with H₂O (5 mL) and extracted with EA (20 mL*3). The combined organic layers were washed with brine (15 mL*2), dried over anhydrous Na₂SO₄, filtered and concentrated under reduced pressure to give a residue. The residue was purified by flash silica gel chromatography (12 g SepaFlash silica flash column; eluent: 6% methanol/dichloromethane at 12 mL/min). Int-D (90 mg, 238.84 μmol, 57.27% yield, 92.740% purity) was obtained as a yellow oil. LCMS (Method G): Rt=0.668 min, [M+H]⁺=350.1. ¹H NMR (400 MHZ, chloroform-d) δ 7.32 (s, 1H), 4.01 (s, 2H), 3.71 (m, 2H), 2.70 (s, 3H), 1.48 (s, 9H).

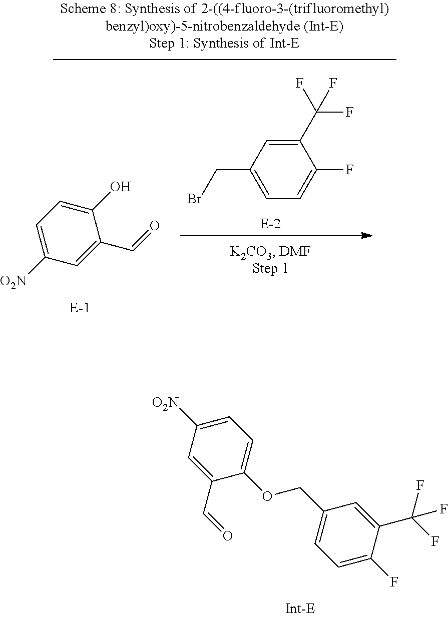

To a solution of E-1 (2 g, 11.97 mmol, 1 eq) and E-2 (3.38 g, 13.16 mmol, 1.1 eq) in DMF (20 mL) was added K₂CO₃ (4.96 g, 35.90 mmol, 3 eq). The mixture was stirred at 60° C. for 2 hrs. The residue was diluted with H₂O (20 mL) and extracted with ethyl acetate (25 mL*2). The combined organic phase was washed with brine (10 mL), dried with anhydrous Na₂SO₄, filtered and concentrated under vacuum. The crude residue was purified through flash silica gel chromatography (40 g SepaFlash silica flash column; eluent: 30-50% ethyl acetate/petroleum ether gradient) to give Int-E (2.5 g, 7.28 mmol, 60.86% yield) as a white solid, 1H NMR (400 MHZ, DMSO-d₆) δ=10.35 (s, 1H), 8.54 (dd, J=2.8, 9.2 Hz, 1H), 8.47 (d, J=3.2 Hz, 1H), 8.05-7.94 (m, 2H), 7.64-7.52 (m, 2H), 5.51 (s, 2H).

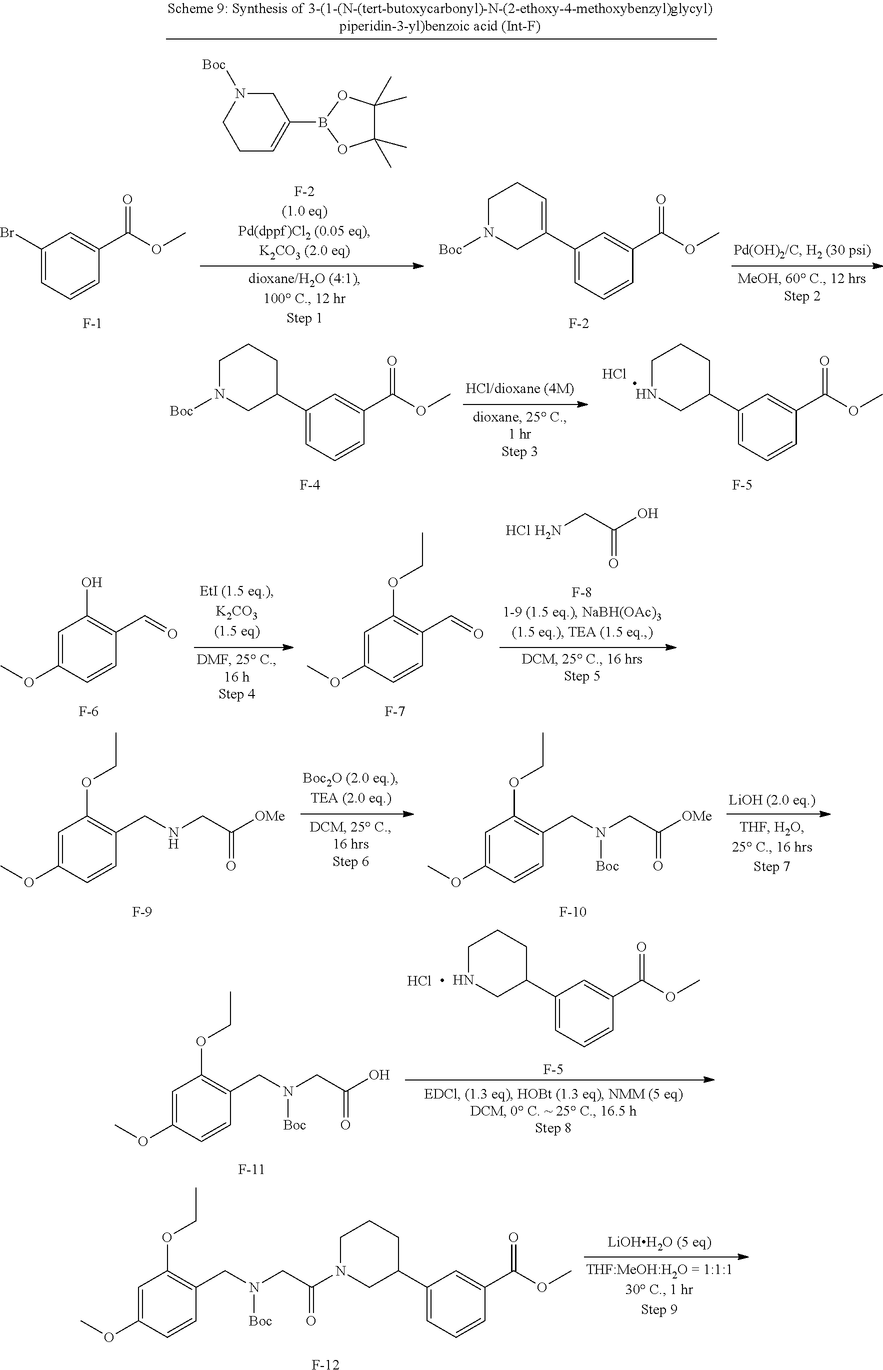

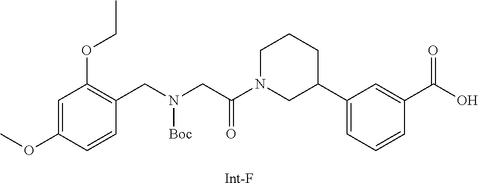

### Step 1: Synthesis of F-3

A mixture of F-1 (105 g, 488.27 mmol, 1 eq), F-2 (150.98 g, 488.27 mmol, 1 eq), Pd(dppf)Cl₂ (17.86 g, 24.41 mmol, 0.05 eq) and K₂CO₃ (134.96 g, 976.55 mmol, 2 eq) in dioxane (840 mL) and H₂O (210 mL) was degassed and purged with N₂ 3 times, and then the mixture was stirred at 100° C. for 12 hr under an N₂ atmosphere. The reaction was quenched by water (1 L), extracted with EA (1 L*3). The combined organic layers were concentrated to give a residue. The residue was divided into two parts and each part was purified by flash silica gel chromatography (: 330 g SepaFlash silica flash column; eluent: 0-6% ethyl acetate/petroleum ether; gradient at 150 mL/min). Eluted fractions were concentrated to give F-3 (150 g, 463.17 mmol, 94.86% yield, 98% purity) as a colorless oil. LCMS (Method E): Rt=0.614 min, [M+Na]⁺=340.1. ¹H NMR (400 MHZ, CDCl₃) δ 8.06 (s, 1H), 7.95 (d, J=7.6 Hz, 1H), 7.56 (br d, J=7.6 Hz, 1H), 7.46-7.34 (m, 1H), 6.28 (m, 1H), 4.30 (br s, 2H), 3.93 (s, 3H), 3.57 (m, 2H), 2.34 (m, 2H), 1.51 (s, 9H).

### Step 2: Synthesis of F-4

A mixture of F-3 (75 g, 236.31 mmol, 1 eq) and Pd(OH)₂/C (8.30 g, 11.82 mmol, 20% purity, 0.05 eq) in MeOH (1 L) was degassed and purged with H₂ 3 times, and then the mixture was stirred at 60° C. for 12 hr under H₂ (20 psi). The reaction mixture was filtered by diatomite (50 g), the filter cake washed with MeOH (100 mL*3) and the filtrate was concentrated to afford F-4 (75 g, 234.82 mmol, 99.37% yield, 99% purity) as a colorless oil. LCMS (Method E): Rt=0.612 min, [M+Na]⁺=342.1. ¹H NMR (400 MHz, CDCl₃) δ 8.02-7.85 (m, 2H), 7.52-7.34 (m, 2H), 4.26-4.12 (m, 2H), 3.92 (s, 3H), 2.84-2.67 (m, 3H), 2.02 (br s, 1H), 1.85-1.74 (m, 1H), 1.73-1.61 (m, 2H), 1.48 (s, 9H).

### Step 3: Synthesis of F-5

To a solution of F-4 (150 g, 469.64 mmol, 1 eq) in dioxane (750 mL) was added HCl/dioxane (4 M, 750 mL, 6.39 eq). The mixture was stirred at 25° C. for 1 hr. The reaction mixture was concentrated to give F-5 (108 g, 409.64 mmol, 87.22% yield, 97% purity, HCl salt) as a white solid. LCMS (Method E): Rt=0.371 min, [M+H]⁺=220.1. ¹H NMR (400 MHZ, DMSO-d₆) δ 9.45-8.97 (m, 2H), 7.94-7.80 (m, 2H), 7.69-7.56 (m, 1H), 7.54-7.46 (m, 1H), 3.86 (s, 3H), 3.32-3.20 (m, 2H), 3.15-3.01 (m, 2H), 2.92 (br t, J=10.6 Hz, 1H), 1.94-1.65 (m, 4H).

### Step 4: Synthesis of F-7

To a solution of F-6 (180 g, 1.18 mol, 1 eq) in DMF (900 mL) was added K₂CO₃ (245.26 g, 1.77 mol, 1.5 eq) and EtI (276.78 g, 1.77 mol, 141.94 mL, 1.5 eq). The resulting mixture was allowed to reach 25° C., and stirred for 16 hr. The reaction mixture was quenched with water (4 L) and extracted by EA (1 L*3), and the combined organic layer were washed with brine (3 L*2) and concentrated under reduced pressure to afford the crude product. The crude product was diluted with EA (200 mL) and recrystallized to afford F-7 (210 g, 1.17 mol) as a white solid. LCMS (Method E): Rt: 0.500 min, [M+H]⁺=181.1. ¹H NMR (400 MHZ, CDCl₃) δ 10.34 (s, 1H), 7.82 (d, J=8.4 Hz, 1H), 6.58-6.49 (m, 1H), 6.44 (d, J=2.0 Hz, 1H), 4.13 (q, J=6.8 Hz, 2H), 3.87 (s, 3H), 1.48 (t, J=6.8 Hz, 3H).

### Step 5: Synthesis of F-9

To a solution of F-7 (150 g, 832.41 mmol, 1 eq) in DCM (1500 mL) was added F-8 (156.77 g, 1.25 mol, 1.5 eq) and TEA (126.35 g, 1.25 mol, 173.79 mL, 1.5 eq). The resulting mixture was stirred for 0.5 hr at 25° C., then NaBH(OAc)₃ (264.63 g, 1.25 mol, 1.5 eq) was slowly added into the system under ice-bath. The whole system was then allowed to reach 25° C., and stirred for another 8 hr. The reaction mixture was quenched with water (3000 mL), the organic layer was separated and washed with brine (1000 mL). The combined organic phase was dried over anhydrous Na₂SO₄, and concentrated under reduced pressure to afford F-9 (290 g, crude) as a colorless oil.

### Step 6: Synthesis of F-10

To a solution of F-9 (290 g, crude, 1 eq) in DCM (2000 mL) was added TEA (231.71 g, 2.29 mol, 318.72 mL, 2.0 eq) and Boc₂O (499.75 g, 2.29 mol, 526.05 mL, 2.0 eq). The system was allowed to reach 25° C., and stirred for 16 hr. The reaction mixture was quenched with water (6000 mL) and extracted with EA (1500 mL*3). The combined organic layer was concentrated under reduced pressure to afford the crude product. The crude product was purified by silica gel chromatography (330 g SepaFlash silica flash column*4. eluent: 5-15% EA/PE) and the eluent was combined and concentrated to give F-10 (240 g, 679.10 mmol, 59.31% yield, 99% purity) as a colorless oil. LCMS (Method E): Rt: 0.623 min, LCMS: Rt: 0.598 min, [M+Na]⁺=376.1. ¹H NMR (400 MHz, CDCl₃) δ 7.25-7.09 (m, 1H), 6.51-6.33 (m, 2H), 4.52-4.35 (m, 2H), 4.04-3.86 (m, 4H), 3.79 (d, J=3.6 Hz, 3H), 3.70 (d, J=2.8 Hz, 3H), 1.46 (d, J=20.0 Hz, 9H), 1.39 (q, J=6.8 Hz, 3H).

### Step 7: Synthesis of F-11

To a solution of F-10 (120 g, 339.55 mmol, 1 eq) in THF (600 mL) was added LiOH·H₂O (14.25 g, 339.55 mmol, 1 eq) and H₂O (600 mL) in an ice-bath. The resulting mixture was then allowed to reach 25° C., and stirred for 16 hrs. The reaction mixture was concentrated under reduced pressure to remove THF (600 mL). The pH of the residue was adjusted to 6 by addition of aq. HCl (4.0 M). The resulting mixture was then extracted with EA (300 mL*3). The combined organic layer was then concentrated to afford the crude product. The crude product was recrystallized by EA (40 mL) to give F-11 (86 g, 253.40 mmol, 74.63% yield, 99% purity) as a white solid. LCMS (Method E): Rt=0.521 min, [M+Na]⁺=362.2. ¹H NMR (400 MHz, CDCl₃) δ 10.76-9.53 (m, 1H), 7.26-7.09 (m, 1H), 6.50-6.39 (m, 2H), 4.55-4.35 (m, 2H), 4.13-3.88 (m, 4H), 3.80 (s, 3H), 1.47 (br d, J=12.6 Hz, 9H), 1.43-1.37 (m, 3H).

### Step 8: Synthesis of F-12

To a solution of F-11 (113.86 g, 335.50 mmol, 1.1 eq) in DCM (800 mL) was added EDCI (76.01 g, 396.50 mmol, 1.3 eq), HOBt (53.58 g, 396.50 mmol, 1.3 eq) and NMM (154.25 g, 1.52 mol, 167.66 mL, 5 eq) at 0° C. The mixture was stirred at 0° C. for 0.5 hr. Then F-5 (78 g, 305.00 mmol, 1 eq, HCl salt) was added into the system. The reaction mixture was allowed to reach 25° C., and stirred for another 16 hrs. The reaction was quenched with water (300 mL) at 0° C., and extracted with DCM (100 mL*3). The combined organic layers were concentrated to afford a residue. The residue was divided into two parts and each part was purified by flash silica gel chromatography (: 330 g SepaFlash silica flash column; eluent of 0-32% ethyl acetate/petroleum ether; gradient at 150 mL/min). The eluent was combined and concentrated to give F-12 (149 g, 275.60 mmol, 90.36% yield, 99% purity) as a colorless oil. LCMS (Method E): Rt=0.622 min, [M+H]⁺=541.3. ¹H NMR (400 MHZ, CDCl₃) δ 7.96-7.82 (m, 2H), 7.50-7.34 (m, 2H), 7.19-7.07 (m, 1H), 6.50-6.37 (m, 2H), 4.83-4.65 (m, 1H), 4.58-4.37 (m, 2H), 4.24-4.15 (m, 1H), 4.07-3.95 (m, 3H), 3.93 (s, 3H), 3.80 (br d, J=8.8 Hz, 4H), 3.10-2.93 (m, 1H), 2.86-2.68 (m, 1H), 2.67-2.52 (m, 1H), 2.09 (br s, 1H), 1.84 (br d, J=12.8 Hz, 1H), 1.79-1.67 (m, 1H), 1.63 (br d, J=5.2 Hz, 1H), 1.47 (br d, J=5.0 Hz, 9H), 1.43-1.34 (m, 3H).

### Step 9: Synthesis of Int-F

To a solution of F-12 (149 g, 275.60 mmol, 1 eq) in THF (500 mL) and MeOH (500 mL) was added a solution of LiOH·H₂O (57.83 g, 1.38 mol, 5 eq) in H₂O (500 mL). The mixture was stirred at 30° C. for 1 hr. The reaction mixture was concentrated under reduced pressure to remove the THF and MeOH at 30° C., and then cooled to 0° C. via ice-bath. After reaching 0° C., the pH was adjusted to 6 by addition of 1 N aq. HCl. The resulting mixture was extracted with EA (500 mL+3), and the combined organic layer was dried over anhydrous Na₂SO₄ and concentrated to afford Int-F (140 g, 263.19 mmol, 95.50% yield, 99% purity) as white solid. LCMS (Method E): Rt=0.566 min, [M+Na]⁺=549.3. ¹H NMR (400 MHZ, CDCl₃) δ 13.18-12.63 (m, 1H), 7.94-7.74 (m, 2H), 7.65-7.35 (m, 2H), 7.06 (m, 1H), 6.62-6.37 (m, 2H), 4.62-4.16 (m, 3H), 4.07-3.91 (m, 4H), 3.72 (br d, J=9.2 Hz, 4H), 3.16-2.95 (m, 1H), 2.77-2.56 (m, 2H), 1.95-1.85 (m, 1H), 1.75 (br d, J=9.2 Hz, 2H), 1.54-1.24 (m, 13H).

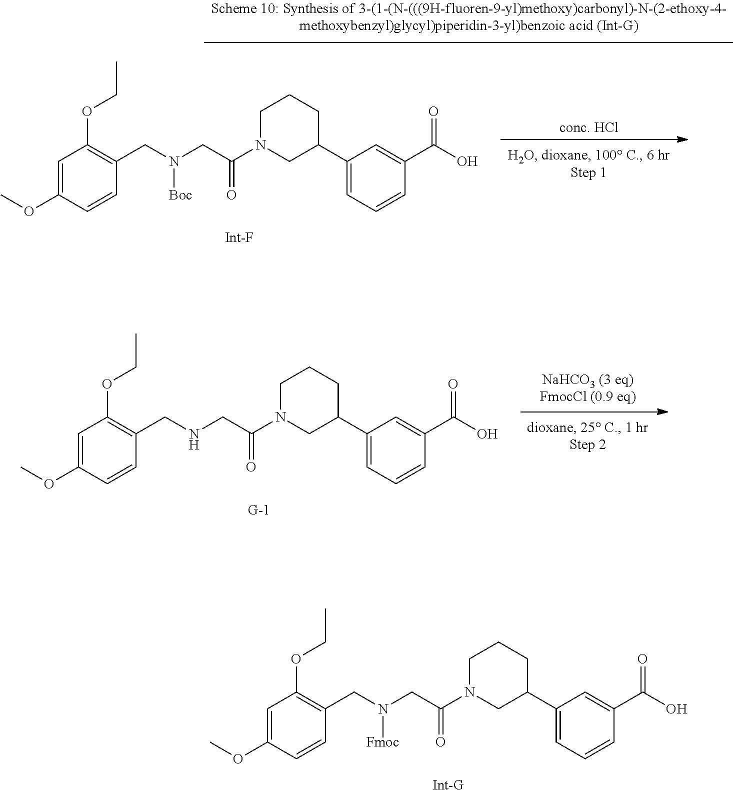

### Step 1: Synthesis of G-1

To a solution of conc. HCl (12 M, 7.99 mL, 1.01 eq) in H₂O (500 mL) and dioxane (50 mL) was added Int-F (50 g, 94.95 mmol, 1 eq). The mixture was stirred at 100° C. for 6 hr. The reaction mixture was cooled to 25° C., and used into next step directly. LCMS (Method E): Rt=0.444 min, [M+H]⁺=427.2.

### Step 2: Synthesis of Int-G

To the reaction mixture of G-1 was added dioxane (450 mL) and NaHCO₃ (23.93 g, 284.80 mmol, 11.08 mL, 3 eq), and then FmocCl (22.10 g, 85.44 mmol, 0.9 eq) was added. The mixture was stirred at 25° C. for 1 hr. The reaction mixture was adjusted to pH 6 with 1 N aq. HCl (70 mL). The resulting mixture was extracted with EA (500 mL*3), and the combined organic layer was dried over anhydrous Na₂SO₄ and concentrated to give Int-G (64 g, 86.81 mmol, two steps yield: 91.45%, 91.3% purity) as white solid. LCMS: Rt=0.607 min, [M+H]⁺=649.3. ¹H NMR (400 MHZ, DMSO-d₆) δ 13.06-12.64 (m, 1H), 7.95-7.78 (m, 4H), 7.64-7.58 (m, 1H), 7.52 (br d, J=6.4 Hz, 2H), 7.48-7.35 (m, 3H), 7.29 (m, 2H), 7.02-6.68 (m, 1H), 6.58-6.49 (m, 1H), 6.49-6.30 (m, 1H), 4.50-4.15 (m, 6H), 4.12-3.88 (m, 4H), 3.73 (br d, J=8.0 Hz, 3H), 3.64-3.58 (m, 1H), 3.16-2.89 (m, 1H), 2.75-2.54 (m, 2H), 1.96-1.90 (m, 1H), 1.83-1.60 (m, 2H), 1.53-1.34 (m, 1H), 1.29 (m, 3H).

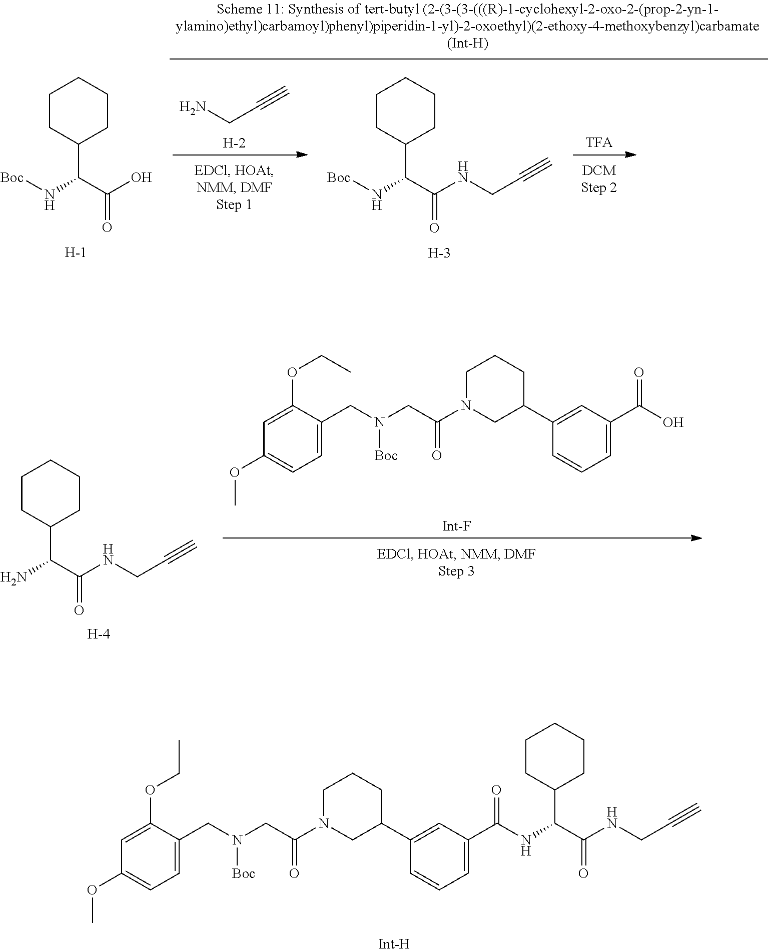

### Step 1: Synthesis of H-3

To a solution of H-1 (500 mg, 1.94 mmol, 1 eq) in DMF (5 mL) was added EDCI (1.12 g, 5.83 mmol, 3 eq), HOAt (264.47 mg, 1.94 mmol, 271.81 μL, 1 eq) and NMM (982.68 mg, 9.72 mmol, 1.07 mL, 5 eq) at 25° C. for 15 min. Then H-2 (139.13 mg, 2.53 mmol, 161.78 μL, 1.3 eq) was added at 25° C. The resulting mixture was stirred at 25° C. for 45 min. EA (4 mL) and water (4 mL) were added and the layers were separated. The aqueous phase was extracted with EA (4 mL×2), dried over Na₂SO₄, filtered, and concentrated under vacuum. The residue was purified by column chromatography (SiO₂, PE/EA=1/0 to 0/1, Rf=0.5 PE/EA=2:1) and the eluent was concentrated to give H-3 (400 mg, 1.36 mmol, 69.93% yield) as a white solid. ¹H NMR (400 MHZ, CHLOROFORM-d) δ=6.19 (s, 1H), 5.08-4.94 (m, 1H), 4.09-3.99 (m, 2H), 3.93-3.85 (m, 1H), 2.26-2.21 (m, 1H), 1.77 (d, J=3.2 Hz, 5H), 1.45 (s, 9H), 1.26-0.92 (m, 6H).

### Step 2: Synthesis of H-4

To a solution of H-3 (150 mg, 509.53 μmol, 1 eq) in DCM (1 mL) was added TFA (460.50 mg, 4.04 mmol, 300.00 μL, 7.93 eq). The mixture was stirred at 25° C. for 0.5 hr. The mixture was concentrated under vacuum. The crude product was used in the next step without purification. H-4 (100 mg, 324.36 μmol, 63.66% yield, TFA) was obtained as a white solid. ¹H NMR (400 MHZ, chloroform-d) δ 7.46-7.44 (m, 1H), 4.05-4.01 (m, 2H), 3.99 (d, J=3.2 Hz, 1H), 2.27-2.26 (m, 1H), 1.93-1.77 (m, 4H), 1.77-1.61 (m, 3H), 1.30-1.06 (m, 6H).

### Step 3: Synthesis of Int-H

To a solution of Int-F (153.73 mg, 291.93 μmol, 1 eq) in DMF (2 mL) was added EDCI (279.82 mg, 1.46 mmol, 5 eq), HOAt (79.47 mg, 583.85 μmol, 81.67 μL, 2 eq) and NMM (295.28 mg, 2.92 mmol, 320.95 μL, 10 eq) at 25° C., and the mixture was stirred at 25° C. for 0.5 hr. Then H-4 (90 mg, 291.93 μmol, 1 eq, TFA) was added to the mixture at 25° C., and the reaction mixture was stirred at 25° C. for 1 hr. DCM (4 mL) and water (4 mL) were added and the layers were separated. The aqueous phase was extracted with DCM (5 mL×2), dried over Na₂SO₄, filtered, and concentrated to give a residue. The residue was purified by column chromatography (SiO₂, PE/EA=1/0 to 0/1) and the eluent was concentrated to give Int-H (175 mg, 241.70 μmol, 82.79% yield, 97.077% purity) as colorless oil. LCMS (Method D): Rt=0.546 min, [M+H]⁺=703.6. SFC (Method J): Rt=0.1.809 min, 2.024 min. ¹H NMR (400 MHZ, chloroform-d) δ 7.64 (s, 2H), 7.37 (s, 1H), 7.19-7.08 (m, 1H), 7.01-6.93 (m, 1H), 6.42 (s, 2H), 4.69 (d, J=12.0 Hz, 1H), 4.56-4.38 (m, 3H), 4.19-4.06 (m, 2H), 4.01-3.97 (m, 4H), 3.78 (d, J=9.2 Hz, 4H), 3.13-2.98 (m, 2H), 2.64-2.52 (m, 1H), 2.21 (s, 1H), 2.03 (d, J=4.0 Hz, 3H), 1.89-1.73 (m, 6H), 1.58 (s, 2H), 1.46 (d, J=4.0 Hz, 9H), 1.42-1.33 (m, 3H), 1.33-1.02 (m, 6H).

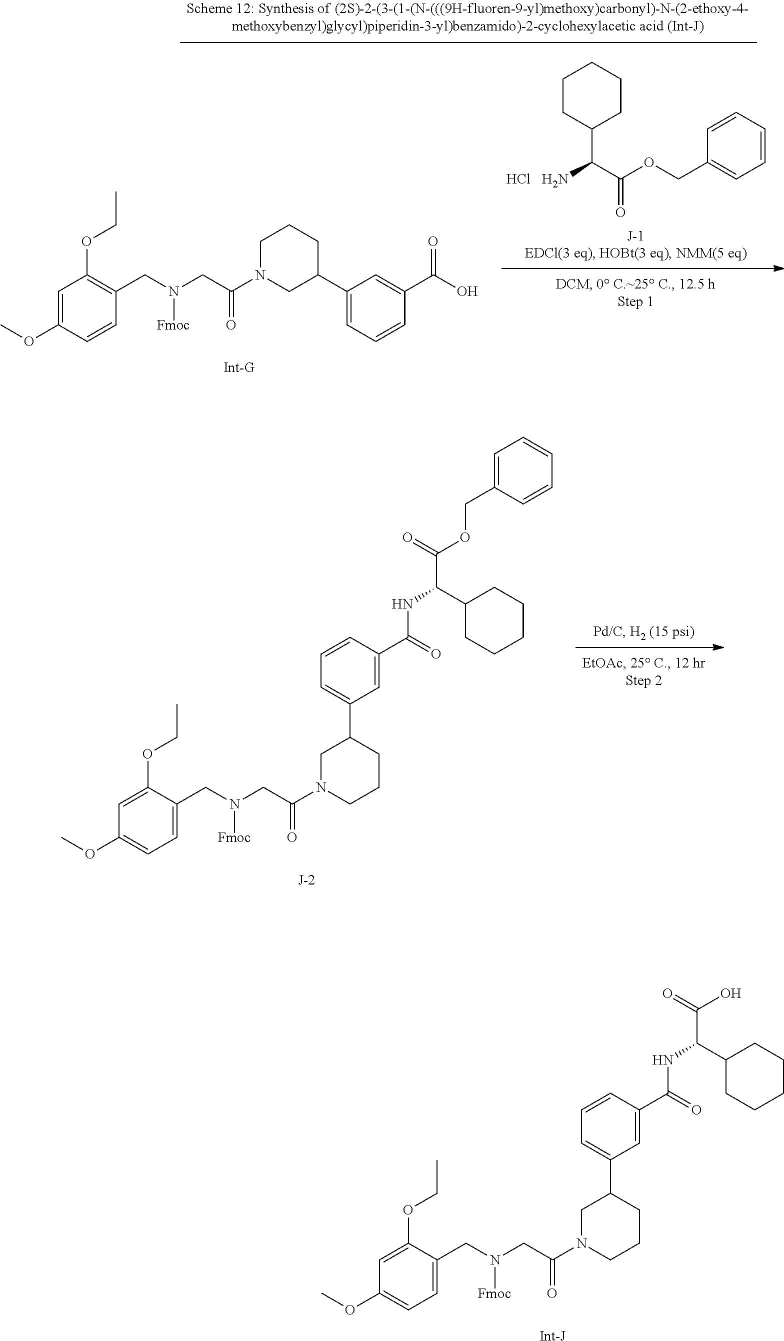

### Step 1: Synthesis of J-2

To a solution of Int-G (34 g, 46.12 mmol, 32.80 μL, 1 eq) in DCM (340 mL) was added EDCI (26.52 g, 138.36 mmol, 3 eq), HOBt (18.70 g, 138.36 mmol, 3 eq) and NMM (23.33 g, 230.60 mmol, 25.35 mL, 5 eq) at 0° C. The mixture was stirred at 0° C. for 0.5 hr. Then J-1 (17.02 g, 59.96 mmol, 1.3 eq, HCl salt) was added into the system. The reaction mixture was allowed to reach 25° C., and stirred for another 12 hrs. The reaction was quenched with water (300 mL). The resulting mixture was extracted by DCM (200 mL*3) and the combined organic layer was dried over anhydrous Na₂SO₄ and concentrated to give a residue. The residue was purified by flash silica gel chromatography (330 g SepaFlash silica flash column. eluent: 0-70% ethyl acetate/petroleum ether gradient at 120 mL/min). The eluent was concentrated under reduce pressure to give J-2 (36 g, 39.36 mmol, 85.34% yield, 96% purity) as white solid. LCMS (Method E): Rt=0.735 min, [M+H]⁺=878.7. SFC: Rt=2.459 min, 5.477 min. ¹H NMR (400 MHZ, DMSO-d₆) δ 8.73-8.53 (m, 1H), 7.93-7.81 (m, 2H), 7.74 (br s, 2H), 7.65-7.49 (m, 2H), 7.48-7.21 (m, 11H), 6.99-6.67 (m, 1H), 6.57-6.28 (m, 2H), 5.23-5.05 (m, 2H), 4.49-4.29 (m, 4H), 4.29-4.16 (m, 3H), 4.13-3.95 (m, 4H), 3.73 (d, J=10.0 Hz, 3H), 3.67-3.53 (m, 1H), 3.15-2.85 (m, 1H), 2.73-2.55 (m, 2H), 1.96-1.83 (m, 2H), 1.81-1.53 (m, 7H), 1.50-1.34 (m, 1H), 1.33-1.24 (m, 3H), 1.16-0.96 (m, 4H).

### Step 2: Synthesis of Int-J

To a flask purged with nitrogen was added Pd/C (2.18 g, 10% purity) then EtOAc (500 mL) was added into the system under inert atmosphere. Then J-2 (36 g, 39.36 mmol, 1 eq) was added into the system. The system was degassed and purged with H₂, then the reaction mixture was stirred at 25° C. under H₂ atmosphere (15 psi) for 12 hr. The reaction mixture was filtered by celite (10 g), the filter cake was washed with EA (50 mL*3) and the filtrate was concentrated to give Int-J (30 g, 35.41 mmol, 89.96% yield, 93% purity) as a white solid. LCMS (Method E): Rt=0.649 min, [M+H]⁺=788.4. ¹H NMR (400 MHz, DMSO-d₆) δ12.82-12.43 (m, 1H), 8.54-8.33 (m, 1H), 7.94-7.81 (m, 2H), 7.76 (br s, 2H), 7.66-7.50 (m, 2H), 7.49-7.35 (m, 4H), 7.35-7.20 (m, 2H), 7.02-6.65 (m, 1H), 6.58-6.31 (m, 2H), 4.50-4.16 (m, 7H), 4.07-3.90 (m, 4H), 3.73 (d, J=8.6 Hz, 3H), 3.69-3.53 (m, 1H), 3.18-2.88 (m, 1H), 2.75-2.54 (m, 2H), 1.97-1.81 (m, 2H), 1.73 (br d, J=12.8 Hz, 6H), 1.64-1.54 (m, 1H), 1.52-1.34 (m, 1H), 1.33-1.25 (m, 3H), 1.17-1.02 (m, 4H).

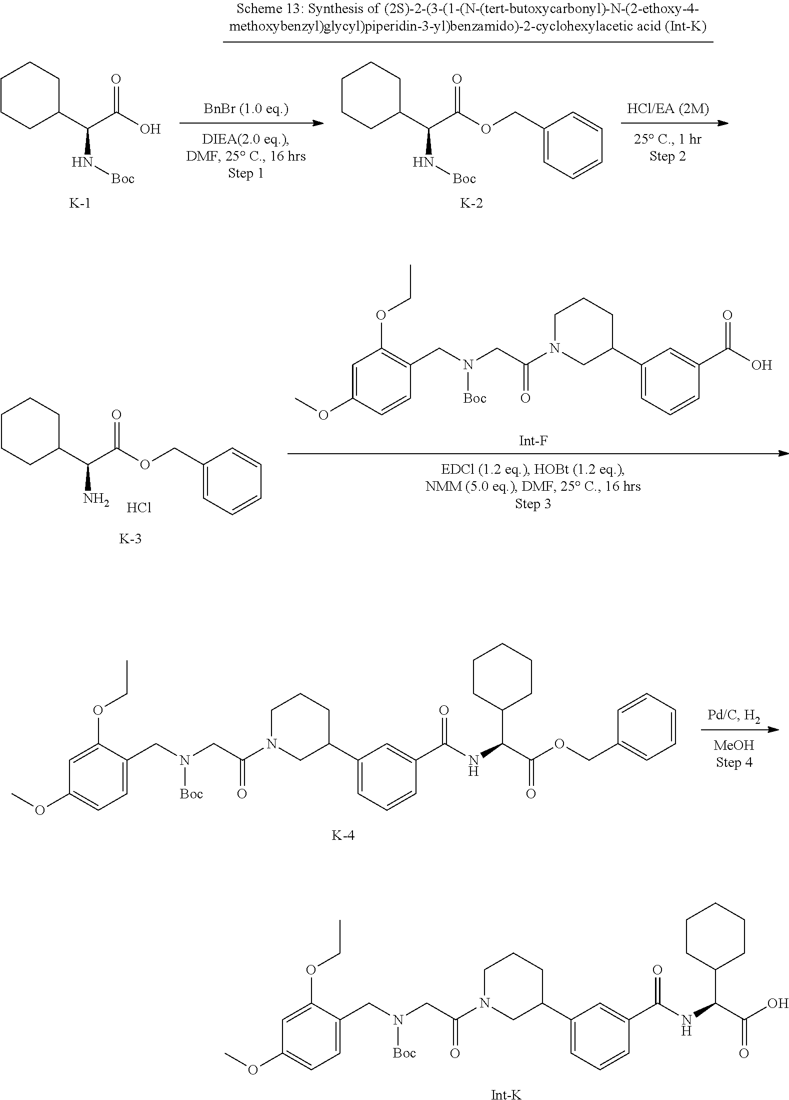

### Step 1: Synthesis of K-2

To a solution of K-1 (50 g, 194.31 mmol, 1 eq) in DMF (500 mL) was added DIEA (50.23 g, 388.61 mmol, 67.69 mL, 2 eq) and BnBr (33.23 g, 194.31 mmol, 23.08 mL, 1 eq). The reaction mixture was allowed to reach 25° C., and stirred for 16 hr. The reaction was quenched with water (2 L), then extracted with EA (500 mL*3). The combined organic layer was dried over anhydrous Na₂SO₄ and then concentrated under reduced pressure (via vacuum pump) to afford the crude product. The crude product was purified by flash silica gel chromatography (330 g SepaFlash silica flash column*2, eluent: 5-15% EA/PE) and the eluent was concentrated to give K-2 (50 g, 133.26 mmol, 68.58% yield, 92.60% purity) as a white solid. LCMS (Method E): Rt=0.658 min, [M+Na]⁺=370.3. ¹HNMR: (400 MHZ, CDCl₃) δ 7.43-7.30 (m, 5H), 5.25-5.10 (m, 2H), 5.07-4.98 (m, 1H), 4.26 (br dd, J=4.9, 8.9 Hz, 1H), 1.85-1.51 (m, 7H), 1.44 (s, 9H), 1.30-1.10 (m, 4H).

### Step 2: Synthesis of K-3

K-2 (25 g, 71.95 mmol, 1 eq) was dissolved in EtOAc (125 mL) and added HCl/EtOAc (4N, 125 mL) was added drop wise. The resulting mixture was stirred at 25° C. for 1 hr. The reaction mixture was concentrated via vacuum pump to give K-3 (25 g, HCl salt form) as white solid. LCMS (Method E): Rt=0.468 min, [M+H]⁺=2482. ¹H NMR (400 MHZ, CDCl₃) δ 8.926 (s, 3H), 7.46-7.30 (m, 5H), 5.34-5.16 (m, 2H), 2.14-2.01 (m, 2H), 1.89-1.70 (m, 3H), 1.69-1.56 (m, 2H), 1.51-1.02 (m, 7H).

### Step 3: Synthesis of K-4

To a solution Int-F (20 g, 37.98 mmol, 1 eq) in DMF (250 mL) was added EDCI (9.46 g, 49.37 mmol, 1.3 eq), HOBt (6.67 g, 49.37 mmol, 1.3 eq) and NMM (19.21 g, 189.89 mmol, 20.88 mL, 5 eq) at 0° C., and the resulting mixture was stirred for 0.5 hr before K-3 (10.78 g, 37.98 mmol, 1.0 eq, HCl) was introduced into the system. The reaction mixture was quenched with water (2 L) and extracted with EA (500 mL*3). The combined organic layer was washed with brine (800 mL*2) and dried over anhydrous Na₂SO₄ and then concentrated under reduced pressure (via vacuum pump) to afford the crude product. The crude was purified by flash silica-gel chromatography (330 g SepaFlash silica flash column*3, eluent: 55-70% EA/PE) and the eluent was concentrated to give K-4 (20 g, 24.34 mmol, 64.09% yield, 92% purity) as colorless oil: LCMS (Method E): Rt=0.734 min, [M+Na]⁺=778.5. ¹H NMR (400 MHZ, CDCl₃) δ 7.75-7.57 (m, 2H), 7.38-7.31 (m, 7H), 7.24-7.05 (m, 1H), 6.79-6.58 (m, 1H), 6.49-6.36 (m, 2H), 5.25-5.13 (m, 2H), 4.85-4.77 (m, 1H), 4.76-4.64 (m, 1H), 4.62-4.33 (m, 2H), 4.04-3.90 (m, 3H), 3.83-3.68 (m, 4H), 3.08-2.94 (m, 1H), 2.80-2.47 (m, 2H), 1.97-1.87 (m, 1H), 1.84-1.52 (m, 10H), 1.51-1.32 (m, 12H), 1.20-0.98 (m, 5H).

### Step 4: Synthesis of Int-K

To a flask purged with nitrogen was added Pd/C (1.00 g, 939.67 μmol, 10% purity) and then EtOAc (100 mL) was added into the system. To the resulting mixture was then added K-4 (10 g, 13.23 mmol, 1 eq). The system was degassed and purged with H₂, and then the reaction mixture was stirred at 25° C. under H2 atmosphere (15 psi) for 1 hr. The reaction mixture was filtered under inert atmosphere. The filter cake was washed with MeOH (50 mL*2). The filtrate was concentrated under reduced pressure to afford Int-K (15 g, 22.07 mmol, 83.41% yield, 97.95% purity) as white solid. LCMS (Method E): Rt=0.605 min, [M+H]⁺=666.5. SFC: Rt1=0.639, Rt2=1.292. ¹H NMR (400 MHz, CDCl₃) δ¹H NMR (400 MHZ, CDCl₃) δ 10.07 (br s, 1H), 7.73-7.66 (m, 1H), 7.65-7.42 (m, 1H), 7.40-7.03 (m, 4H), 6.49-6.33 (m, 2H), 4.79-4.63 (m, 2H), 4.60-4.06 (m, 4H), 4.04-3.88 (m, 3H), 3.65-3.80 (m, 3H), 3.04-2.97 (m, 1H), 2.80-2.48 (m, 2H), 2.03-1.91 (m, 2H), 1.87-1.58 (m, 8H), 1.52-1.33 (m, 12H), 1.24-1.01 (m, 5H).

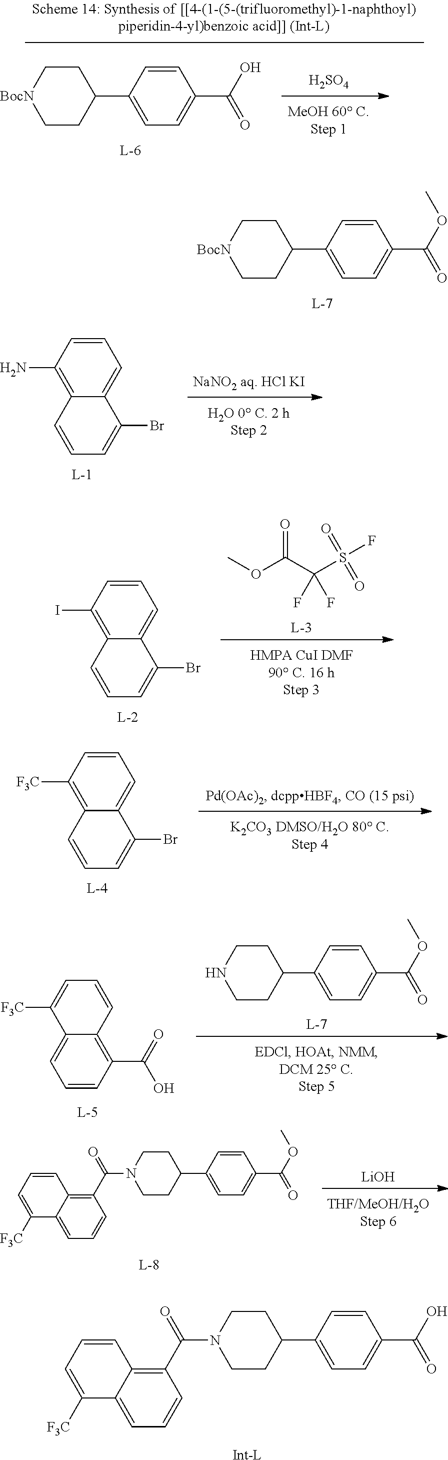

### Step 1: Synthesis of L-7

The mixture of L-6 (45 g, 147.36 mmol, 1 eq) in MeOH (500 mL) and conc. H₂SO₄ (25 mL) was stirred at 70° C. for 12 hr. The reaction was filtered and concentrated under reduced pressure to give L-7 (44 g, 134.60 mmol, 91.34% yield, 97.08% purity, H₂SO₄ salt) as a white solid which was used into the next step without further purification. LCMS (Method E): Rt=0.367 min, [M+H]⁺=220.2. ¹H NMR (400 MHz, DMSO-d₆) δ 8.65-8.19 (m, 2H), 7.93 (d, J=8.4 Hz, 2H), 7.39 (d, J=8.4 Hz, 2H), 3.84 (s, 3H), 3.43-3.38 (m, 1H), 3.34 (s, 1H), 3.09-2.89 (m, 3H), 2.02-1.90 (m, 2H), 1.88-1.73 (m, 2H).

### Step 2: Synthesis of L-2

To a solution of L-1 (85 g, 382.74 mmol, 1 eq) in conc. HCl (900 mL) was added a solution of NaNO₂ (52.81 g, 765.49 mmol, 2 eq) in H₂O (130 mL), the mixture was stirred at 0° C. for 0.5 h, and then a solution of KI (317.68 g, 1.91 mol, 5 eq) in H₂O (250 mL) was added. The mixture was stirred at 0° C. for 0.5 h. The reaction was diluted with H₂O (3.0 L) and extracted with ethyl acetate (1 L*3). The combined organic phase was dried over Na₂SO₄, filtered and concentrated under reduced pressure to give a residue. The residue was purified by recrystallization from MeOH (500 mL) at 25° C. and filtered. The filter cake was dried under vacuum to give L-2 (90 g, 270.30 mmol, 70.62% yield) as a brown solid. ¹H NMR (400 MHz, DMSO-d₆) δ 8.23-8.21 (m, 1H), 8.17 (d, J=8.4 Hz, 1H), 8.06 (d, J=8.4 Hz, 1H), 7.96-7.94 (m, 1H), 7.56-7.52 (m, 1H), 7.42-7.38 (m, 1H).

### Step 3: Synthesis of L-4

A mixture of L-2 (80 g, 240.27 mmol, 1 eq), L-3 (230.79 g, 1.20 mol, 152.84 mL, 5 eq), CuI (77.79 g, 408.45 mmol, 1.7 eq) and N-[bis(dimethylamino)phosphoryl]-N-methyl-methanamine (215.28 g, 1.20 mol, 210.23 mL, 5 eq) in DMF (800 mL) was degassed and purged with N₂ 3 times, and then the mixture was stirred at 90° C. for 16 hr under an N₂ atmosphere. The reaction was cooled to room temperature, then diluted with H₂O (2 L), extracted with ethyl acetate (1 L*3), and the combined organic phase was washed with saturated aqueous NaCl (2 L), dried over Na₂SO₄, filtered and concentrated under reduced pressure to give a residue. The residue was purified by flash silica gel chromatography (660 g SepaFlash silica flash column; eluent: 0-5% ethyl acetate/petroleum ether gradient at 200 mL/min). The eluent was concentrated to afford L-4 (100 g, crude) as a yellow solid. ¹H NMR (400 MHZ, CDCl₃) δ 8.51 (d, J=8.4 Hz, 1H), 8.26-8.13 (m, 1H), 7.95-7.90 (m, 2H), 7.65-7.58 (m, 1H), 7.48-7.44 (m, 1H).

### Step 4: Synthesis of L-5

To a solution of 1-4 (50 g, 181.78 mmol, 1 eq) in DMSO (500 mL) and H₂O (50 mL) was added Pd(OAc)₂ (4.08 g, 18.18 mmol, 0.1 eq), 1,3-bis(dicyclohexylphosphino) propane bis(tetrafluoroborate) (22.26 g, 36.36 mmol, 0.2 eq) and K₂CO₃ (50.25 g, 363.55 mmol, 2 eq) under N₂. The suspension was degassed under vacuum and purged with CO three times. The mixture was stirred under CO (20 psi) at 80° C. for 16 hours. The reaction was diluted with H₂O (1 L) and then extracted with ethyl acetate (500 mL*2) to remove impurities. The aqueous phase was adjusted to pH=4 with saturated aqueous citric acid and extracted with EA (1 L*3). The combined organic phase was dried over Na₂SO₄, filtered and concentrated under reduced pressure to give a residue. The residue was purified by flash silica gel chromatography (660 g SepaFlash silica flash column; eluent: 0-60% ethyl acetate/petroleum ether gradient at 200 mL/min). The eluent was concentrated to afford L-5 (30 g, 124.91 mmol, 34.36% yield) as a yellow solid. LCMS: Rt=0.206 min, (M+H)=239.1. ¹H NMR (400 MHZ, DMSO-d₆) δ 13.77-13.13 (m, 1H), 9.18 (d, J=8.8 Hz, 1H), 8.35 (d, J=8.8 Hz, 1H), 8.30 (d, J=7.2 Hz, 1H), 8.13 (d, J=7.2 Hz, 1H), 7.92-7.79 (m, 2H).

### Step 5: Synthesis of L-8

To a solution of L-5 (30 g, 124.91 mmol, 1 eq) and L-7 (39.64 g, 124.91 mmol, 1.0 eq, H₂SO₄ salt) in DCM (300 mL) was added EDCI (71.84 g, 374.72 mmol, 3 eq), NMM (63.17 g, 624.54 mmol, 68.66 mL, 5 eq) and HOAt (25.50 g, 187.36 mmol, 26.21 mL, 1.5 eq). The mixture was stirred at 25° C. for 1 hr. The reaction was diluted with H₂O (500 mL) and extracted with ethyl acetate (500 mL*3) and the combined organic phase was washed with saturated aqueous NaCl (500 mL), dried over Na₂SO₄, filtered and concentrated under reduced pressure to give a residue. The residue was purified by flash silica gel chromatography (660 g SepaFlash silica flash column; eluent: 0-100% ethyl acetate/petroleum ether gradient at 200 mL/min). The eluent was concentrated to afford L-8 (30 g, 61.16 mmol, 48.97% yield, 90% purity) as a yellow solid. LCMS (Method E): Rt=0.629 min, [M+H]⁺=442.2. ¹H NMR (400 MHZ, CDCl₃) δ 8.25 (d, J=8.4 Hz, 1H), 8.21-8.03 (m, 1H), 8.01-7.98 (m, 2H), 7.96-7.88 (m, 1H), 7.73-7.45 (m, 3H), 7.34-7.23 (m, 2H), 5.17-5.10 (m, 1H), 3.92 (d, J=2.4 Hz, 3H), 3.63-3.41 (m, 1H), 3.22-3.06 (m, 1H), 3.05-2.94 (m, 1H), 2.90-2.83 (m, 1H), 2.18-2.04 (m, 1H), 1.97-1.81 (m, 1H), 1.80-1.68 (m, 1H), 1.63-1.39 (m, 1H).

### Step 6: Synthesis of Int-L

To a solution of L-8 (28 g, 63.43 mmol, 1 eq) in MeOH (200 mL), THF (100 mL) and H₂O (100 mL) was added LiOH·H₂O (7.99 g, 190.29 mmol, 3 eq). The mixture was stirred at 25° C. for 1 hr. The reaction mixture was concentrated under reduced pressure to give a residue. The residue was diluted with H₂O (200 mL) and extracted with ethyl acetate (100 mL*2) to remove impurities. The aqueous phase was adjusted to pH 4 with saturated aqueous citric acid and extracted with EA (200 mL*3). Then the combined organic phase was dried over Na₂SO₄, filtered and concentrated under reduced pressure to give Int-L (26 g, 60.26 mmol, 95.0% yield, 99.05% purity) as a white solid which was used in the next step without purification. LCMS (Method E): Rt=0.553 min, [M+H]⁺=428.2. ¹H NMR (400 MHZ, DMSO-d₆) δ 13.45-12.05 (m, 1H), 8.28-8.02 (m, 3H), 7.89-7.86 (m, 2H), 7.84-7.55 (m, 3H), 7.43-7.39 (m, 2H), 4.88-4.81 (m, 1H), 3.37-3.24 (m, 1H), 3.23-3.06 (m, 1H), 3.05-2.80 (m, 2H), 2.03-1.91 (m, 1H), 1.90-1.70 (m, 1H), 1.67-1.14 (m, 2H).

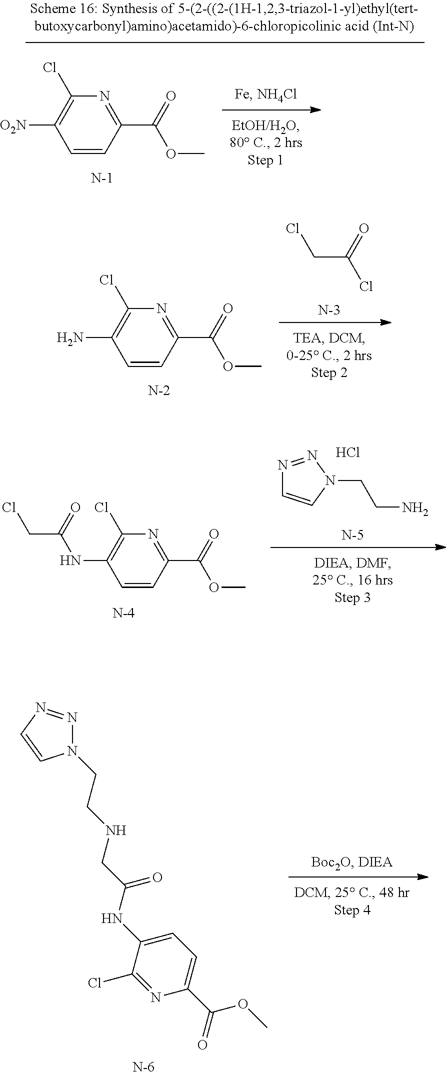

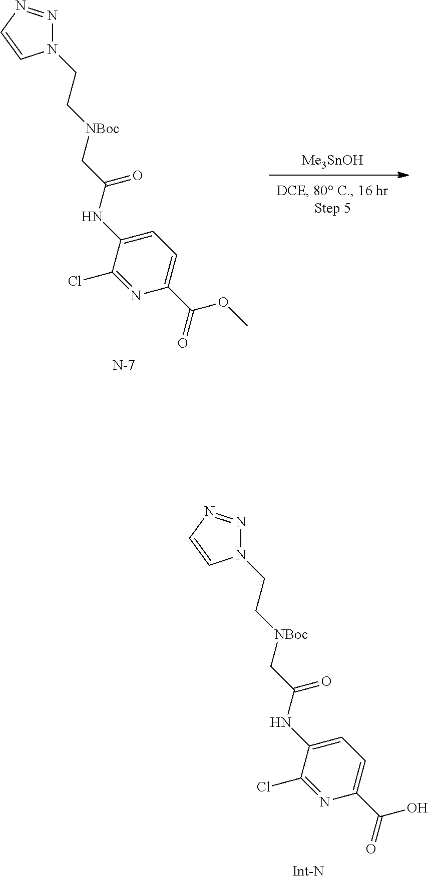

### Step 1: Synthesis of N-2

To a solution of N-1 (60 g, 277.04 mmol, 1 eq), NH₄Cl (37.05 g, 692.59 mmol, 2.5 eq) in EtOH (400 mL) and H₂O (400 mL) was added Fe (77.36 g, 1.39 mol, 5 eq). The mixture was stirred at 80° C. for 1 hr. The reaction mixture was filtered and the filtrate was poured into water (200 mL) and extracted with EA (200 mL*2). The organic layer was washed with brine (20 mL), dried over Na₂SO₄, and concentrated to afford the crude product N-2 (52 g, 268.65 mmol, 96.97% yield, 96.4% purity) as a yellow solid. LCMS (Method E): Rt=0.360 min, [M+Na]⁺=208.9.

### Step 2: Synthesis of N-4

To a solution of N-2 (48 g, 257.24 mmol, 1 eq) and TEA (260.30 g, 2.57 mol, 358.05 mL, 10 eq) in DCM (500 mL) was added N-3 (174.32 g, 1.54 mol, 122.76 mL, 6 eq) at 0° C. Then the mixture was stirred at 25° C. for 16 hr. The reaction mixture was poured into water (100 mL) and extracted with DCM (100 mL*2). The organic layer was washed with brine (10 mL), dried over Na₂SO₄ and concentrated to afford the crude product. The crude product was purified by flash silica gel chromatography (SiO₂, PE:EA=10:1 to 3:1) to give N-4 (36 g, 109.88 mmol, 42.72% yield, 80.3% purity) as a yellow solid. LCMS (Method E): Rt=0.434 min, [M+H]⁺=262.9.

### Step 3: Synthesis of N-6

To a solution of N-4 (35 g, 133.04 mmol, 1 eq), N-5 (39.54 g, 266.08 mmol, 2 eq, HCl) in DMF (400 mL) was added DIEA (85.97 g, 665.20 mmol, 115.87 mL, 5 eq) and was allowed to stir at 25° C. for 16 hr. The reaction mixture was poured into water (600 mL) and extracted with EA (300 mL*4). The combined organic layers were washed with brine (300 mL), dried over Na₂SO₄ and concentrated to give the crude product. The crude product was purified by flash silica gel chromatography (SiO₂, PE:EA=5:1 to 1:1) to give N-6 (24 g, 70.07 mmol, 52.67% yield, 98.9% purity) as a yellow solid. LCMS (Method E): Rt=0.306 min, [M+H]⁺=339.0. ¹H NMR (400 MHZ, DMSO-d₆) δ 8.80 (d, J=8.4 Hz, 1H), 8.17 (d, J=0.8 Hz, 1H), 8.11 (d, J=8.4 Hz, 1H), 7.73 (d, J=0.8 Hz, 1H), 4.54-4.52 (m, 2H), 3.88 (s, 3H), 3.42 (s, 2H), 3.13-3.11 (m, 2H).

### Step 4: Synthesis of N-7

To solution of N-6 (13 g, 38.38 mmol, 1 eq) and DIEA (14.88 g, 115.13 mmol, 20.05 mL, 3 eq) in DCM (130 mL) was added Boc₂O (16.75 g, 76.75 mmol, 17.63 mL, 2 eq). The mixture was stirred at 25° C. for 48 hr. The reaction mixture was concentrated to give the crude product, which was purified by flash silica gel chromatography (SiO₂, PE:EA=10:1 to 1:1) to give N-7 (13.5 g, 26.82 mmol, 69.90% yield, 87.2% purity) as white solid. LCMS (Method E): Rt=0.457 min, [M+H]⁺=439.1.

### Step 5: Synthesis of Int-N

To a solution of N-7 (16 g, 36.46 mmol, 1 eq) in DCE (160 mL) was added hydroxy (trimethyl) stannane (32.96 g, 182.29 mmol, 5 eq) and was allowed to stir at 80° C. for 16 hr. The reaction mixture was filtered and concentrated to give the crude product. The crude product was purified by flash silica gel chromatography (SiO₂, PE:EA=3:1 to 1:1) to give Int-N (15.5 g, 25.72 mmol, 70.55% yield, 70.5% purity) as white solid. LCMS (Method E): Rt=0.421 min, [M+H]⁺=425.2. ¹H NMR (400 MHz, DMSO-d₆) δ 9.79 (br d, J=8.8 Hz, 1H), 8.51-8.27 (m, 1H), 8.16 (s, 1H), 7.95 (br d, J=5.6 Hz, 1H), 7.73 (d, J=9.6 Hz, 1H), 4.56-4.54 (m, 2H), 4.12-4.00 (m, 2H), 3.72-3.70 (m, 2H), 1.39-1.18 (m, 9H).

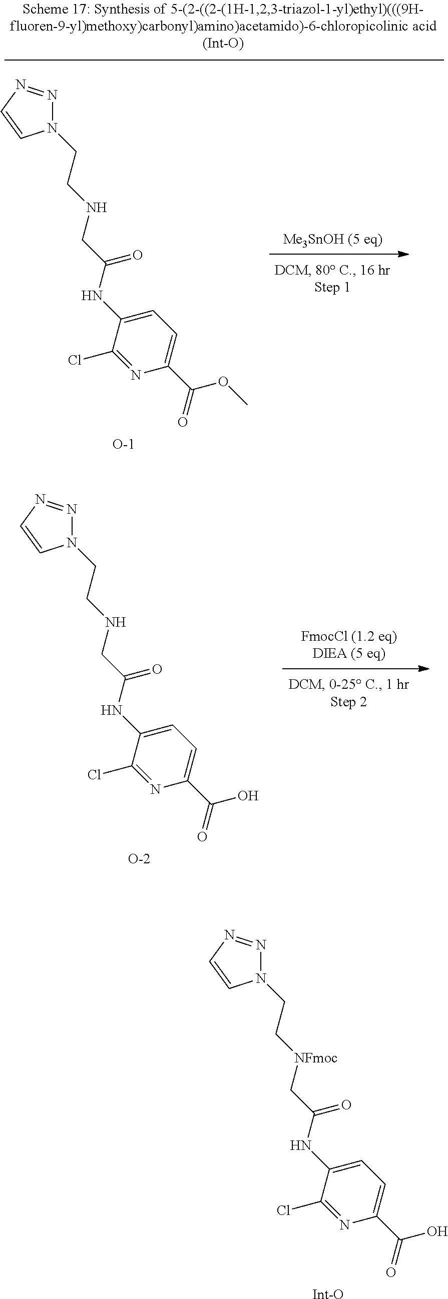

### Step 1: Synthesis of 0-2

To a solution of 0-1 (21 g, 61.99 mmol, 1 eq) in DCE (200 mL) was added hydroxy (trimethyl) stannane (56.05 g, 309.96 mmol, 5 eq). Then the mixture was stirred at 80° C. for 16 hr. The reaction mixture was filtered and the filtrate was concentrated to give the crude product 0-2 (20 g, 46.87 mmol, 75.61% yield, 76.1% purity) as yellow oil. LCMS (Method E): Rt=0.329 min, [M+H]⁺=325.

### Step 2: Synthesis of Int-Q

To solution of O-2 (20 g, 61.59 mmol, 1 eq), DIEA (39.80 g, 307.96 mmol, 53.64 mL, 5 eq) in DCE (200 mL) was added Fmoc-Cl (39.83 g, 153.98 mmol, 2.5 eq) and the mixture was allowed to stir at 0° C. for 1 hr. The reaction mixture was filtered and concentrated to give the crude product. The crude product was purified by flash silica gel chromatography (SiO₂, PE:EA=10:1 to 1:1) to give Int-O (14.8 g, 25.90 mmol, 42.04% yield, 95.7% purity) as light yellow solid. LCMS: Rt=0.493 min, [M+H]⁺=547.2. ¹H NMR (DMSO-d₆, 400 MHz) δ 9.92 (br d, J=15.6 Hz, 1H), 8.50-8.48 (m, 1H), 8.25-7.98 (m, 2H), 7.94-7.71 (m, 4H), 7.68-7.54 (m, 2H), 7.45-7.31 (m, 3H), 7.17-7.15 (m, 1H), 4.60-4.58 (m, 1H), 4.38-4.18 (m, 4H), 4.16-4.01 (m, 2H), 3.79-3.77 (m, 1H), 3.65-3.63 (m, 1H).

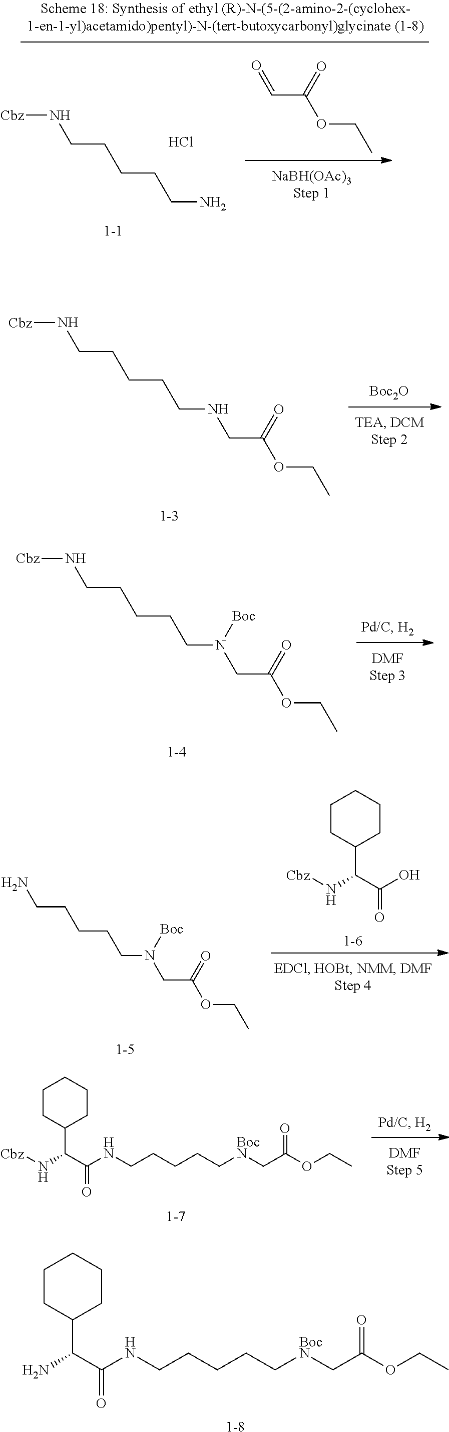

### Step 1: Synthesis of 1-3

1-1 (500 mg, 2.12 mmol, 1 eq) was added into 10% aq. K₂CO₃ (10 mL) and extracted with DCM (5 mL*3). The combined organic layers were dried over anhydrous sodium sulfate, filtered, and concentrated to give a residue. The residue was dissolved in TFE (5 mL), followed by addition of ethyl 2-oxoacetate (432.01 mg, 2.12 mmol, 1 eq) and 4 Å MS (100 mg). The reaction mixture was stirred at 25° C. for 1 h. NaBH(OAc)₃ (1.12 g, 5.29 mmol, 2.5 eq) was then added at 0° C. The reaction mixture was stirred at 25° C. for 1 h. The reaction mixture was filtered to remove solids. The filtrate was poured into 10% aq. K₂CO₃ (10 mL) and extracted with DCM (10 mL*3). The combined organic layers were washed with brine (10 mL), dried over anhydrous sodium sulfate, filtered, and concentrated to give a residue. The residue purified by flash silica gel chromatography (12 g SepaFlash silica flash column; eluent: 0-40% MeOH/ethyl acetate; gradient at 20 mL/min) and the eluent was concentrated to give 1-3 (200 mg, 620.35 μmol, 29.32% yield, 99% purity) as a yellow solid. LCMS (Method E): Rt=0.510 min, [M+H]⁺=323.0.

### Step 2: Synthesis of 1-4

To a solution of 1-3 (200 mg, 620.35 μmol, 1 eq) in DCM (2 mL) was added TEA (125.55 mg, 1.24 mmol, 172.69 μL, 2 eq) and Boc₂O (176.01 mg, 806.46 μmol, 185.27 μL, 1.3 eq). The reaction mixture was stirred at 25° C. for 2 h. The reaction mixture was diluted with DCM (1 mL) and purified by flash silica gel chromatography (20 g silica flash column: eluent of 0-40% ethyl acetate/petroleum ether, gradient at 30 mL/min) to afford 1-4 (190 mg, 427.20 μmol, 68.87% yield, 95% purity) as a colorless oil. LCMS (Method E): Rt=0.590 min, [M+Na]⁺=445.4. ¹H NMR (400 MHz, CDCl₃) δ 7.41-7.29 (m, 5H), 5.21-5.03 (m, 2H), 4.94-4.63 (m, 1H), 4.21-4.16 (m, 2H), 3.99-3.75 (m, 2H), 3.37-3.10 (m, 4H), 1.56-1.50 (m, 4H), 1.50-1.39 (m, 9H), 1.39-1.23 (m, 5H).

### Step 3: Synthesis of 1-5

To a solution of 1-4 (190 mg, 449.69 μmol, 1 eq) in DMF (2 mL) was added Pd/C (100 mg, 93.97 μmol, 10% purity) and the suspension was degassed under vacuum and purged with H₂ (15 psi) three times. The mixture was stirred at 25° C. for 1 hr. The reaction mixture was concentrated under reduced pressure to give 1-5 (120 mg, crude), which was used into the next step without further purification. LCMS (Method E): Rt=0.398 min, [M+H]⁺=289.2.

### Step 4: Synthesis of 1-7

To a solution of 1-5 (700 mg, 2.43 mmol, 1.1 eq) and 1-6 (642.89 mg, 2.21 mmol, 1 eq) in DMF (7 mL) was added EDCI (1.27 g, 6.62 mmol, 3 eq), HOBt (298.17 mg, 2.21 mmol, 1 eq) and NMM (2.23 g, 22.07 mmol, 2.43 mL, 10 eq). The mixture was stirred at 25° C. for 2 hr. The reaction mixture was concentrated under reduced pressure to give the crude product. The product was purified by prep-HPLC (column: Phenomenex luna C18 150 mm*25 mm*10 um; mobile phase: [water (FA):ACN]; gradient: 57%-87% over 10 min) and the eluent was concentrated and then lyophilized to give 1-7 (600 mg, 1.04 mmol, 46.95% yield, 97% purity) as a colorless oil. LCMS (Method E): Rt=0.622 min, [M+H]⁺=562.4. SFC: Rt=1.332 min, ee=97.57%.

### Step 5: Synthesis of 1-8

To a solution of 1-7 (600 mg, 1.07 mmol, 1 eq) in DMF (6 mL) was added Pd/C (300 mg, 281.90 μmol, 10% purity). The suspension was degassed under vacuum and purged with H₂ (15 psi) three times. The mixture was stirred at 25° C. for 1 hr. The reaction mixture was concentrated under reduced pressure to give 1-8 (420 mg, 982.28 μmol, 91.96% yield, 99% purity) as a white solid which was used into the next step without further purification. LCMS (Method E): Rt=0.463 min, [M+H]⁺=428.2.

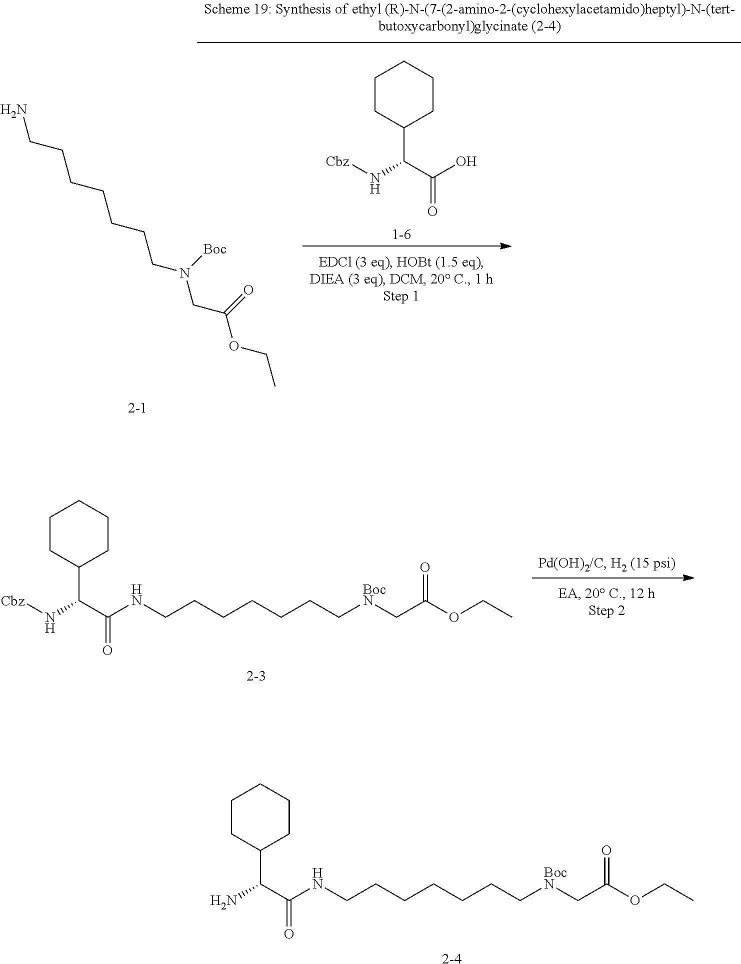

### Step 1: Synthesis of 2-3

To a mixture of 1-6 (202.55 mg, 695.25 μmol, 1.1 eq), HOBt (128.11 mg, 948.06 μmol, 1.5 eq) and EDCI (363.49 mg, 1.90 mmol, 3 eq) in DCM (2 mL) was added DIEA (245.06 mg, 1.90 mmol, 330.27 μL, 3 eq) and the mixture was stirred at 20° C. for 5 min. Then, 2-1 (200 mg, 632.04 μmol, 1 eq) was added. The mixture was stirred at 20° C. for 1 hr. The mixture was then poured into a solution of saturated NaHCO₃ (3 mL) and extracted with DCM (3 mL*3). The combined organic layers were dried over anhydrous Na₂SO₄, filtered, and concentrated to give a residue. The residue was purified by flash silica gel chromatography (8 g SepaFlash silica flash column; eluent: 0-50% ethyl acetate/petroleum ether; gradient at 40 mL/min) and the eluent was concentrated. 2-3 (170 mg, 288.25 μmol, 45.61% yield) was obtained as a white solid. LCMS (Method E): Rt=0.659 min, [M-99]⁺=490.4.

### Step 2: Synthesis of 2-4

To a solution of N-3 (150 mg, 254.34 μmol, 1 eq) in EtOAc (3 mL) was added Pd(OH) 2/C (50 mg, 20% purity) under N₂. The suspension was degassed under vacuum and purged with H₂ several times. The mixture was stirred under H₂ (15 psi) at 20° C. for 12 hours. The mixture was filtered and washed with EA (5 mL*4) and the filtrate was collected. The filtrate was concentrated to give the crude product. 2-4 (100 mg, crude) was obtained as brown oil. LCMS (Method E): Rt=0.325 min, [M+H]⁺=456.3.

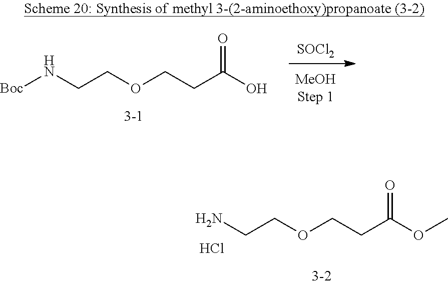

### Step 1: Synthesis of 3-2

To a solution of 3-1 (25 g, 107.18 mmol, 1 eq) in MeOH (250 mL) was added SOCl₂ (15.30 g, 128.61 mmol, 9.34 mL, 1.2 eq) at 0° C. The mixture was stirred at 25° C. for 16 hr. The reaction mixture was filtered and concentrated under reduced pressure to give crude 3-2 (20 g, crude, HCl) as a white solid. ¹H NMR (400 MHZ, CDCl₃) δ 8.50-7.72 (m, 3H), 5.20-4.60 (m, 1H), 3.82-3.77 (m, 4H), 3.70 (s, 3H), 3.27 (s, 2H), 2.67-2.63 (m, 2H).

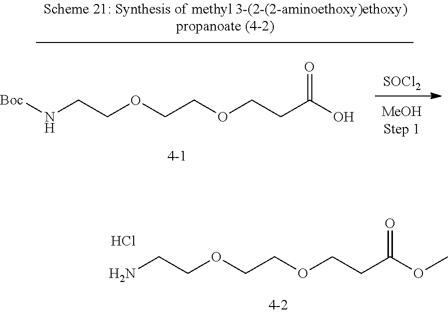 [SMILES: CC(C)(C)OC(=O)NCCOCCOCCC(=O)O ; "SOCl2" ; Cl ; COC(=O)CCOCCOCCN]

### Step 1: Synthesis of 4-2

To a solution of 4-1 (25 g, 90.15 mmol, 1 eq) in MeOH (250 mL) was added SOCl₂ (12.87 g, 108.18 mmol, 7.86 mL, 1.2 eq) at 0° C. The mixture was stirred at 25° C. for 16 hr. The reaction mixture was filtered and concentrated under reduced pressure to give crude 4-2 (27 g, crude, HCl salt) as a white solid. ¹H NMR (400 MHZ, CDCl₃) δ 8.31-7.80 (m, 4H), 4.95 (s, 2H), 3.84-3.81 (m, 2H), 3.78-3.74 (m, 2H), 3.71-3.63 (m, 7H), 3.27 (s, 2H), 2.63-2.59 (m, 2H).

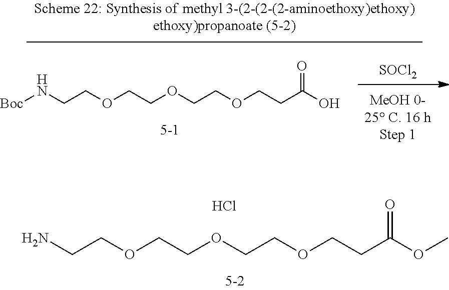 [SMILES: CC(C)(C)OC(=O)NCCOCCOCCOCCC(=O)O ; "SOCl2" ; COC(=O)CCOCCOCCOCCN ; Cl]

### Step 1: Synthesis of 5-2

To a solution of 5-1 (27 g, 84.02 mmol, 1 eq) in MeOH (270 mL) was added SOCl₂ (11.99 g, 100.82 mmol, 7.32 mL, 1.2 eq) at 0° C. The mixture was stirred at 0-25° C. for 16 h. The mixture was concentrated under reduced pressure to give a residue. The crude product was used in the next step without further purification. 5-2 (25 g) was obtained as a colorless oil. ¹H NMR (400 MHZ, CDCl₃) δ 8.01 (s, 2H), 3.77 (m, 2H), 3.69 (m, 2H), 3.67-3.59 (m, 7H), 3.57 (s, 4H), 3.26-3.14 (m, 2H), 2.56 (m, 2H).

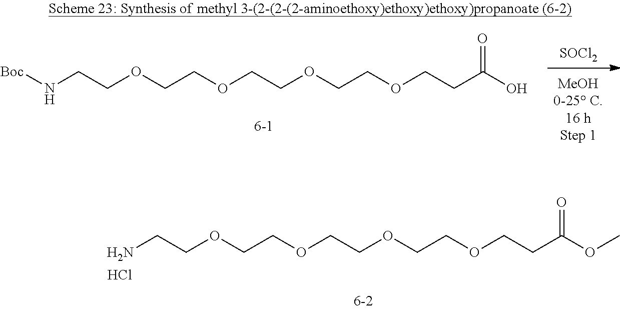 [SMILES: CC(C)(C)OC(=O)NCCOCCOCCOCCOCCC(=O)O ; "SOCl2" ; COC(=O)CCOCCOCCOCCOCCN ; Cl]

### Step 1: Synthesis of 6-2

To a solution of 6-1 (27 g, 73.89 mmol, 1 eq) in MeOH (270 mL) was added SOCl₂ (10.55 g, 88.67 mmol, 6.44 mL, 1.2 eq) at 0° C. The mixture was stirred at 0-25° C. for 16 h. The mixture was concentrated under reduced pressure to give a residue. The crude product was used in the next step without further purification. 6-2 (25.6 g, HCl salt) was obtained as a yellow oil. ¹H NMR (400 MHZ, CDCl₃) δ 7.98 (s, 2H), 3.78 (m, 2H), 3.71 (m, 2H), 3.67-3.54 (m, 15H), 3.21-3.20 (d, J=4.4 Hz, 2H), 2.56 (m, 2H).

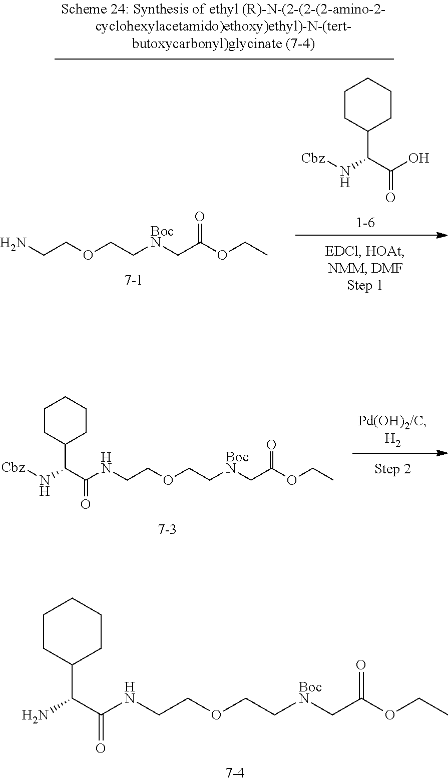

### Step 1: Synthesis of 7-3

To a solution of 1-6 (280 mg, 961.07 μmol, 1 eq), 7-1 (279.05 mg, 961.07 μmol, 1 eq), EDCI (368.48 mg, 1.92 mmol, 2 eq), and HOAt (65.41 mg, 480.54 μmol, 67.22 μL, 0.5 eq) in DCM (3 mL) was added NMM (486.05 mg, 4.81 mmol, 528.31 μL, 5 eq). The mixture was stirred at 25° C. for 1 hr. The reaction mixture was then poured into water (10 mL), extracted with DCM (10 mL*2), and the organic layer was washed with brine (10 mL), dried over Na₂SO₄, and concentrated to afford the crude product. The crude product was purified by flash silica gel column (SiO₂, PE:EA=5:1 to 1:1) to afford 7-3 (270 mg, 478.99 μmol, 49.84% yield, 99% purity) as a colorless oil. LCMS (Method E): Rt=0.612 min, [M+H]⁺=564.5. SFC: Rt=1.904 min, ee=95.1%. ¹H NMR (400 MHZ, CDCl₃) δ 7.44-7.30 (m, 5H), 5.11 (s, 2H), 4.29-4.09 (m, 4H), 4.08-3.93 (m, 2H), 3.69-3.35 (m, 8H), 1.87-1.68 (m, 5H), 1.54-1.38 (m, 9H), 1.32-1.24 (m, 6H), 1.14 (br s, 2H). Step 2: Synthesis of 7-4

To a solution of 7-3 (270 mg, 478.99 μmol, 1 eq) in MeOH (3 mL) was added Pd(OH) 2/C (200 mg, 284.83 μmol, 20% purity). The mixture was stirred at 25° C. for 16 hr under H₂ (15 psi). The reaction mixture was filtered and the filtrate was concentrated under vacuum to give 7-4 (150 mg, crude) as a colorless oil. LCMS (Method E): Rt=0.448 min, [M+H]⁺=430.6.

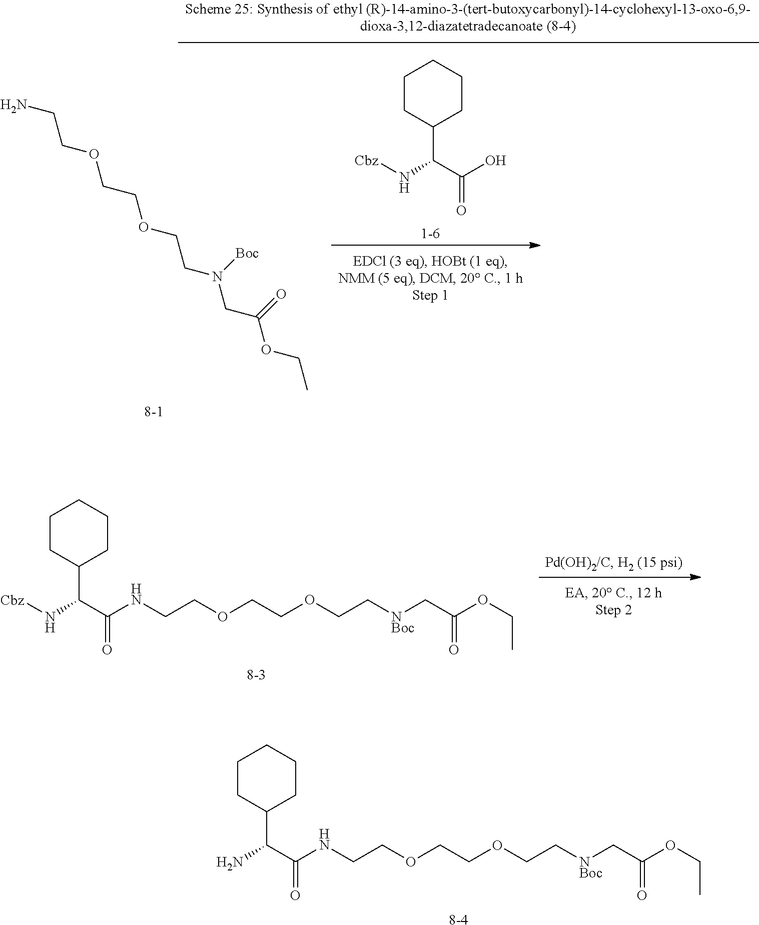

### Step 1: Synthesis of 8-3

To a mixture of 1-6 (287.50 mg, 986.82 μmol, 1.1 eq), EDCI (515.93 mg, 2.69 mmol, 3 eq) and HOBt (121.22 mg, 897.11 μmol, 1 eq) in DCM (3 mL) was added NMM (453.70 mg, 4.49 mmol, 493.15 μL, 5 eq) and the mixture was stirred at 20° C. for 10 min. Then, 8-1 (300 mg, 897.11 μmol, 1 eq) was added. The mixture was stirred at 20° C. for 50 min. The mixture was poured into a solution of saturated NaHCO₃ (10 mL) and extracted with DCM (10 mL*3). The combined organic layers were dried over anhydrous Na₂SO₄, filtered, and concentrated to give a residue. The residue was purified by flash silica gel chromatography (40 g SepaFlash silica flash column; eluent: 0-10% MeOH/DCM; gradient at 60 mL/min) and the eluent was concentrated to give the product. The product was repurified by prep-HPLC (column: YMC-Actus Triart C18 150 mm*30 mm*7 um; mobile phase: [water (FA):ACN]; gradient: 63%-93% ACN over 10 min) and concentrated to remove MeCN, followed by adjustment of the pH to 7-8 with a solution of saturated NaHCO₃ and extraction with DCM (10 mL*3). The combined organic layers were dried over anhydrous Na₂SO₄, filtered and concentrated to afford 8-3 (300 mg, 493.64 μmol, 55.03% yield) as an off-white solid. LCMS (Method E): Rt=0.606 min, [M+H]⁺=608.4. SFC: Rt=1.972 min. ¹H NMR (400 MHz, methanol-d₄) δ 7.56-7.25 (m, 5H), 5.08 (s, 2H), 4.24-4.12 (m, 2H), 4.03 (d, J=8.0 Hz, 2H), 3.92 (d, J=6.4 Hz, 1H), 3.61-3.49 (m, 7H), 3.49-3.32 (m, 5H), 1.84-1.59 (m, 6H), 1.55-1.38 (m, 9H), 1.35-0.98 (m, 8H).

### Step 2: Synthesis of 8-4

To a solution of 8-3 (300 mg, 493.64 μmol, 1 eq) in EtOAc (5 mL) was added Pd(OH) 2/C (100 mg, 20% purity) under N₂. The suspension was degassed under vacuum and purged with H₂ several times. The mixture was stirred under H₂ (15 psi) at 20° C. for 12 hours. The mixture was filtered and washed with EA (5 mL*3). The filtrate was concentrated to give 8-4 (200 mg, crude) as a colorless oil. LCMS (Method E): Rt=0.463 min, [M+H]⁺=474.4.

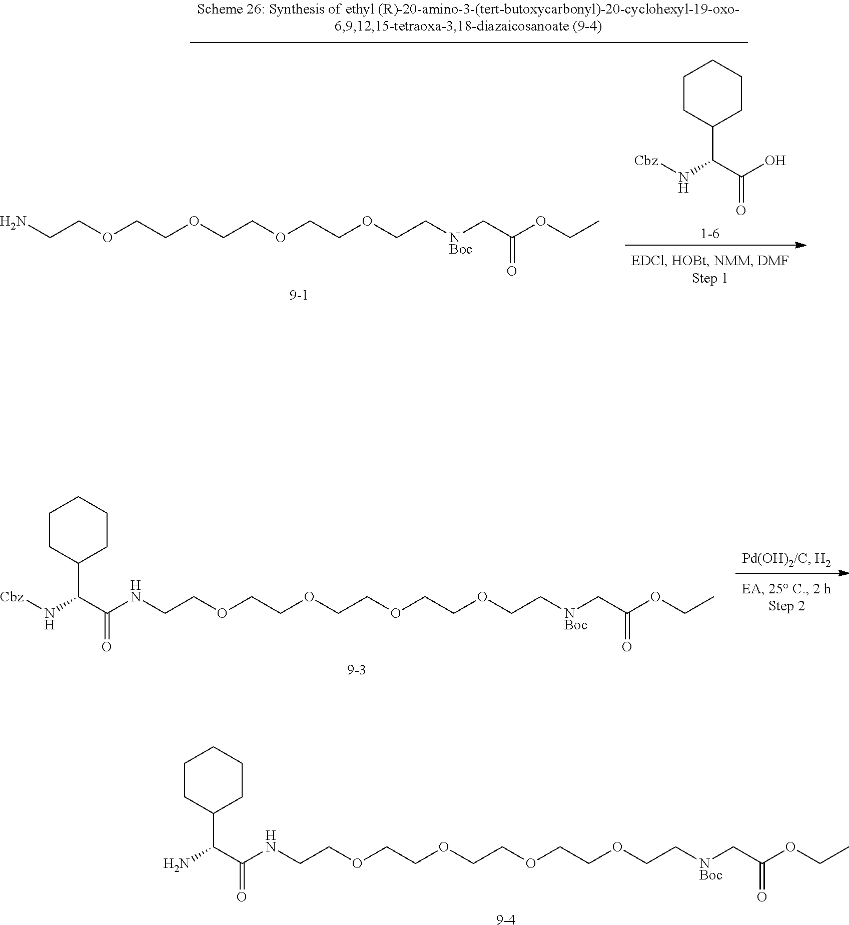

### Step 1: Synthesis of 9-3

To a solution of 9-1 (110 mg, 260.35 μmol, 1 eq) and 1-6 (83.44 mg, 286.38 μmol, 1.1 eq) in DMF (1 mL) was added EDCI (99.82 mg, 520.70 μmol, 2 eq), NMM (131.67 mg, 1.30 mmol, 143.12 μL, 5 eq) and HOBt (17.59 mg, 130.17 μmol, 0.5 eq). The mixture was stirred at 25° C. for 2 hr. The reaction mixture was quenched with water (10 mL). The mixture was extracted with EA (5 mL*3) and dried over anhydrous Na₂SO₄. The mixture was filtered and the filtrate was concentrated under vacuum. The residue was purified by flash silica gel chromatography (20 g SepaFlash silica flash column; eluent: 60-100% ethyl acetate/petroleum ether; gradient at 20 mL/min), 9-3 (150 mg, 215.57 μmol, 82.80% yield, 100% purity) was obtained as a white solid. LCMS (Method E): Rt=0.600 min, [M+H]⁺=696.5. ¹H NMR (400 MHZ, DMSO-d₆) δ=8.02-7.96 (m, 1H), 7.40-7.28 (m, 5H), 7.26-7.20 (m, 1H), 5.01 (s, 2H), 4.14-4.08 (m, 2H), 3.95 (d, J=8.4 Hz, 2H), 3.85-3.80 (m, 1H), 3.56-3.34 (m, 18H), 3.31-3.22 (m, 1H), 3.19-3.11 (m, 2H), 1.70-1.47 (m, 6H), 1.43-1.30 (m, 9H), 1.22-1.17 (m, 3H), 1.13-0.92 (m, 4H).

### Step 2: Synthesis of 9-4

To a mixture of Pd(OH)₂/C (50 mg, 50% purity) in EtOAc (2 mL) was added a solution of 9-3 (150 mg, 215.57 μmol, 1 eq) in EtOAc (2 mL) under N₂. The suspension was degassed under vacuum and purged with H2 several times. The mixture was stirred under H₂ (15 psi) at 25° C. for 2 hours. The reaction mixture was washed by MeOH 15 mL (5 mL*3) and then filtered to give a residue. The residue was used in the next step without further purification. 9-4 (110 mg, 195.83 μmol, 90.84% yield) was obtained as a white solid. ¹H NMR (400 MHZ, DMSO-d₆) δ 7.87-7.83 (m, 1H), 4.13-4.07 (m, 2H), 3.94 (d, J=8.4 Hz, 2H), 3.53-3.44 (m, 14H), 3.42-3.38 (m, 2H), 3.36 (s, 2H), 3.27-3.18 (m, 2H), 3.16 (d, J=5.2 Hz, 2H), 2.90 (d, J=5.2 Hz, 1H), 1.69-1.47 (m, 7H), 1.41-1.31 (m, 9H), 1.21-1.16 (m, 3H), 1.14-0.92 (m, 4H).

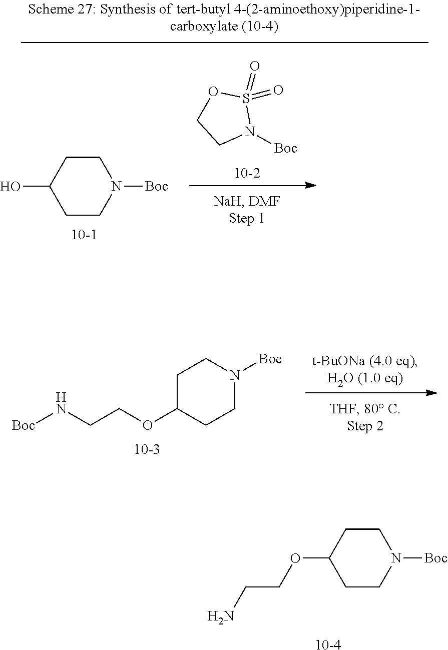

### Step 1: Synthesis of 10-3

To a solution of NaH (4.77 g, 119.25 mmol, 60% purity, 1.2 eq) in DMF (80 mL) was added a solution of 10-1 (20 g, 99.37 mmol, 1 eq) in DMF (120 mL) dropwise at 0° C. The mixture was stirred for 0.5 h at 25° C., and then 10-2 (23.29 g, 104.34 mmol, 1.05 eq) was added to the mixture. The mixture was stirred at 25° C. for 1.5 h. The reaction mixture was quenched with a saturated NH₄Cl solution (200 mL) and extracted with EA (200 mL*2). The organic layer was washed with a citric acid solution (30 mL*3) and dried with anhydrous Na₂SO₄, filtered and concentrated under vacuum. The aqueous phase was adjusted to pH 4 with citric acid and extracted with EA (200 mL*3). The organic layer was dried with anhydrous Na₂SO₄, filtered and concentrated under vacuum. The residue was triturated with PE/EA=20/1 (200 mL), filtered and the filter cake was washed with PE (50 mL*3), the filter cake was dried under vacuum to give 10-3 (31.6 g, 91.74 mmol, 92.32% yield) as white solid. ¹H NMR (400 MHZ, CDCl₃) δ 4.87 (br s, 1H), 3.74 (br d, J=6.4 Hz, 2H), 3.62-3.39 (m, 3H), 3.34-3.17 (m, 2H), 3.14-3.00 (m, 2H), 1.80 (br dd, J=2.9, 6.5 Hz, 2H), 1.55-1.36 (m, 20H).

### Step 2: Synthesis of 10-4

To a solution of 10-3 (10 g, 29.03 mmol, 1 eq) in THF (100 mL) was added t-BuONa (11.16 g, 116.13 mmol, 4 eq) and H₂O (523.02 mg, 29.03 mmol, 523.02 μL, 1 eq). The mixture was stirred at 80° C. for 2 hr. The mixture was concentrated under vacuum. The residue was purified by column chromatography (SiO₂, DCM/MeOH=10/1) to give 10-4 (26.5 g, 108.46 mmol, 93.40% yield) as yellow oil. LCMS (Method G): Rt=0.474 min, [M+Na]⁺=267.2. ¹H NMR (400 MHZ, DMSO-d₆) δ 3.67-3.54 (m, 2H), 3.49-3.32 (m, 1H), 3.37-3.34 (m, 2H), 3.02 (br d, J=9.2 Hz, 2H), 2.64-2.61 (m, 2H), 1.80-1.72 (m, 2H), 1.46-1.25 (m, 11H).

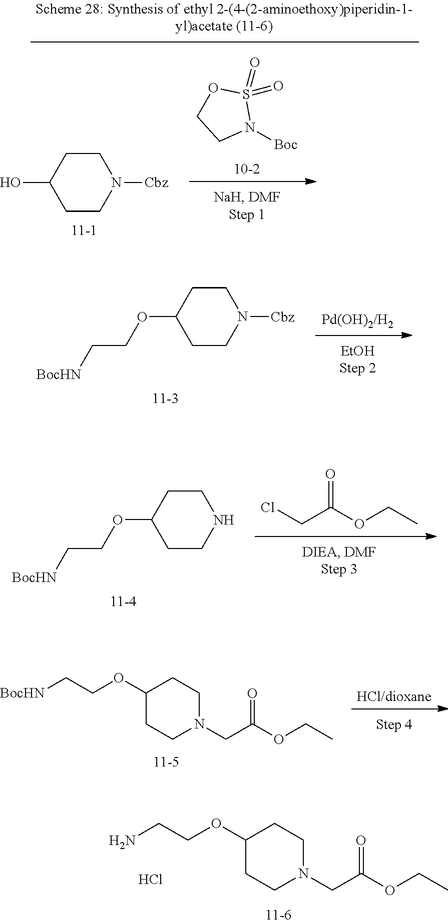

### Step 1: Synthesis of 11-3

In a 500 mL three-neck round-bottomed flask, NaH (18.36 g, 459.03 mmol, 60% purity; 1.2 eq) was dissolved in DMF (500 mL), 11-1 (90 g, 382.53 mmol, 1 eq) in DMF (500 mL) was added dropwise at 0° C. Then the reaction mixture was stirred at 25° C. for 0.5 h. Then 10-2 (89.67 g, 401.65 mmol, 1.05 eq) was added to the above mixture at 0° C., and stirred for another 2.5 h. The reaction mixture was quenched with a saturated NH₄Cl solution (3000 mL) and extracted with MTBE (1500 mL). Then the aqueous phase was extracted with EA (1500 mL*3). The combined organic phase was dried over anhydrous Na₂SO₄, filtered and concentrated to give 11-3 (118 g, 296.20 mmol, 77.43% yield, 95% purity) as yellow oil. 1H NMR (400 MHZ, CDCl₃) δ 7.41-7.29 (m, 5H), 5.13 (s, 2H), 4.86 (br s, 1H), 3.90-3.72 (m, 2H), 3.62-3.43 (m, 3H), 3.30 (br d, J=5.0 Hz, 2H), 3.26-3.17 (m, 2H), 1.82 (br s, 2H), 1.60-1.49 (m, 2H), 1.45 (s, 9H).

### Step 2: Synthesis of 11-4

To a solution of Pd(OH)₂/C (8.02 g, 11.41 mmol, 20% purity, 0.054 eq) in EtOH (800 mL) was added 11-3 (80 g, 211.38 mmol, 1 eq) under N₂. Then the reaction mixture was stirred at 25° C. for 16 hr under H₂ (50 psi) after degassing and refilling with H₂ three times. The reaction mixture was filtered through a pad of celite and concentrated to give 11-4 (58 g, crude) as a yellow oil. LCMS (Method E): Rt=0.534 min, [M+H]⁺⁺=245.1. ¹H NMR (400 MHZ, DMSO-d₆) δ 6.81-6.67 (m, 1H), 3.42-3.32 (m, 3H), 3.09-3.01 (m, 2H), 3.00-2.92 (m, 2H), 2.65-2.55 (m, 2H), 1.90-1.76 (m, 2H), 1.37 (s, 10H).

### Step 3: Synthesis of 11-5

To a solution of 11-4 (30 g, 122.78 mmol, 1 eq) in ACN (300 mL) was added DIEA (31.74 g, 245.57 mmol, 42.77 mL, 2 eq) and ethyl 2-chloroacetate (18.06 g, 147.34 mmol, 15.70 mL, 1.2 eq). Then the reaction mixture was stirred at 40° C. for 16 hr. The reaction mixture was concentrated to afford a residue. The mixture was triturated with EA (200 mL), and the remaining residue was triturated again with EA (100 mL). The precipitate was filtered and the filtrate (desired product) was concentrated. The crude product was diluted with EA (200 mL) and saturated citric acid was added to adjust the pH to 3-4. The mixture was stirred for 0.5 h. The organic phase was separated and the aqueous solution was basified with a NaHCO₃ solution to a pH of 8-9. The aqueous phase was then extracted with DCM (500 mL*2). The combined organic phase was concentrated to give 11-5 (35 g, 105.93 mmol, 86.27% yield) as yellow oil. LCMS (Method E): Rt=0.421 min, [M+H]⁺=331.3. ¹H NMR (400 MHZ, DMSO-d₆) δ 6.79-6.63 (m, 1H), 4.13-3.95 (m, 3H), 3.39-3.30 (m, 6H), 3.29-3.20 (m, 1H), 3.16 (s, 2H), 3.09-2.97 (m, 2H), 2.73-2.64 (m, 2H), 2.30-2.18 (m, 2H), 1.99 (s, 1H), 1.85-1.72 (m, 2H), 1.37 (s, 10H), 1.24-1.11 (m, 4H).

### Step 4: Synthesis of 11-6

11-5 (35 g, 105.93 mmol, 1 eq) was dissolved in HCl/dioxane (35 mL). The mixture was stirred at 25° C. for 12 hr. The reaction mixture was concentrated under reduced pressure to remove solvent and give a residue. The residue was triturated with MTBE (500 mL) at 25° C. for 30 min and filtered. The filter cake was dried to give 11-6 (31 g, crude, HCl salt) as a white solid. LCMS (Method B): Rt=0.210 min, [M+H]⁺⁺=231.4. ¹H NMR (400 MHZ, D₂O) δ 4.29 (q, J=7.1 Hz, 2H), 4.09 (br s, 2H), 3.89-3.67 (m, 4H), 3.20 (br s, 3H), 3.52-3.15 (m, 3H), 2.20-2.11 (m, 1H), 2.36-2.10 (m, 1H), 2.08-1.75 (m, 1H), 1.81 (br d, J=10.8 Hz, 1H), 1.27 (t, J=7.2 Hz, 3H).

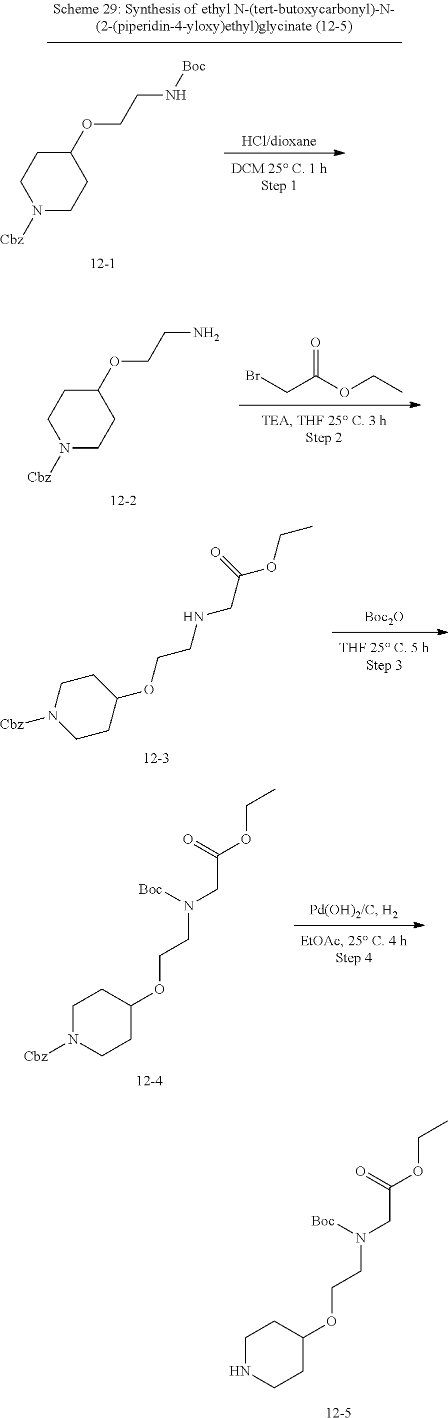

### Step 1: Synthesis of 12-2

To a solution of 12-1 (19 g, 50.20 mmol, 1 eq) in DCM (200 mL) was added HCl/dioxane (4 M in dioxane, 200.00 mL, 15.94 eq). The mixture was stirred at 25° C. for 1 h. The mixture was concentrated under reduced pressure to give a residue. The crude product was used in the next step without further purification. 12-2 (14 g, crude, HCl salt) was obtained as white solid. LCMS (Method E): Rt=0.417 min, [M+H]⁺=297.4.

### Step 2: Synthesis of 12-3

To a solution of 12-2 (12 g, 43.11 mmol, 1 eq) in THF (120 mL) was added TEA (8.72 g, 86.22 mmol, 12.00 mL, 2 eq) and ethyl 2-bromoacetate (7.92 g, 47.42 mmol, 5.25 mL, 1.1 eq). The mixture was stirred at 25° C. for 3 h. The crude product 12-3 (14.1 g, 38.69 mmol, 89.74% yield) was obtained as a colorless oil, which was used in the next step without further purification. LCMS (Method E): Rt=0.423 min, [M+H]⁺=365.2.

### Step 3: Synthesis of 12-4

To a solution of 12-3 (14 g, 38.42 mmol, 1 eq) in THF (140 mL) was added Boc₂O (16.77 g, 76.83 mmol, 17.65 mL, 2 eq). The mixture was stirred at 25° C. for 5 h. The reaction mixture was quenched by addition of water (100 mL) at 25° C., and then extracted with EA (20 mL*3). The organic layer was dried over Na₂SO₄, filtered and concentrated under reduced pressure to give a residue. The residue was purified by flash silica gel chromatography (120 g SepaFlash silica flash column; eluent: 0-80% ethyl acetate/petroleum ether gradient at 100 mL/min), 12-4 (10.5 g, 20.75 mmol, 54.01% yield, 91.8% purity) was obtained as a colorless oil. LCMS: Rt=0.626 min, [M+Na]⁺=487.2.

### Step 4: Synthesis of 12-5

To a solution of Pd(OH)/C (50% purity, 1.00 eq) in EtOAc (20 mL) was added 12-4 (10 g, 21.53 mmol, 1 eq) in EtOAc (80 mL) under N₂. The suspension was degassed under vacuum and purged with H₂ several times. The mixture was stirred under H₂ (15 psi) at 25° C. for 4 h. The reaction mixture was filtered to remove Pd(OH)₂/C, and the filtrate was concentrated. The crude product was used in the next step without further purification. 12-5 (28.6 g) was obtained as a colorless oil. ¹H NMR (400 MHZ, CDCl₃) δ 4.17 (m, 2H), 4.08-3.97 (m, 2H), 3.63-3.53 (m, 2H), 3.50-3.39 (m, 2H), 3.38-3.28 (m, 1H), 3.11-2.98 (m, 2H), 2.61 (m, 2H), 2.20 (s, 2H), 1.95-1.79 (m, 2H), 1.46-1.36 (m, 9H), 1.29-1.24 (m, 3H).

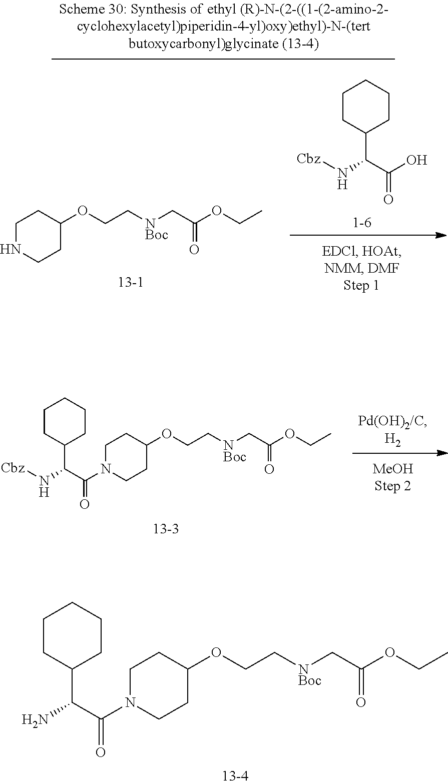

### Step 1: Synthesis of 13-3

To a solution of 1-6 (283 mg, 858.10 μmol, 1 eq), 13-1 (250 mg, 858.10, 1 eq), EDCI (368.48 mg, 1.92 mmol, 2 eq), and HOAt (65.41 mg, 480.54 μmol, 67.22 μL, 0.5 eq) in DCM (3 mL) was added NMM (486.05 mg, 4.81 mmol, 528.31 μL, 5 eq). The mixture was stirred at 25° C. for 1 hr. The reaction mixture was poured into water (10 mL) and extracted with DCM (10 mL*2). The organic layer was washed with brine (10 mL), dried over Na₂SO₄, and concentrated to afford the crude product. The crude product was purified by flash silica gel column (SiO₂, PE:EA=10:1 to 3:1) to yield 13-3 (400 mg, 653.26 μmol, 76.13% yield, 98.6% purity) as a colorless oil. LCMS (Method E): Rt=0.648 min, [M+H]⁺=603.4. SFC: Rt=1.31 min, ee=94.5%.

### Step 2: Synthesis of 13-4

To a solution of 13-3 (400 mg, 662.53 μmol, 1 eq) in MeOH (4 mL) was added Pd(OH) 2/C (232.61 mg, 331.27 μmol, 20% purity). The mixture was stirred at 25° C. for 16 hr under H₂ (15 psi). The reaction mixture was filtered and the filtrate was concentrated under vacuum to yield 13-4 (300 mg, 611.99 μmol, 92.37% yield, 95.8% purity) as a colorless oil, which was used in next step without purification. LCMS (Method E): Rt=0.466 min, [M+H]⁺=470.4.

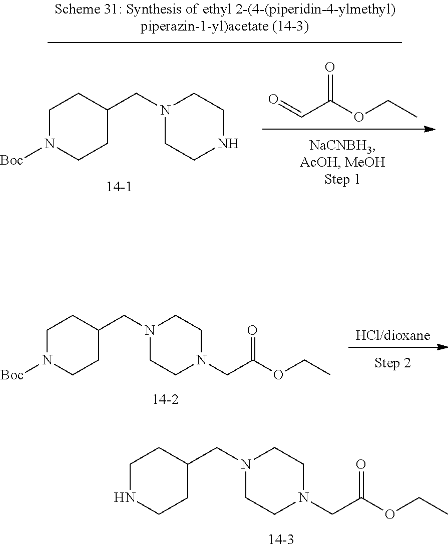

### Step 1: Synthesis of 14-2

To a solution of 14-1 (500 mg, 1.76 mmol, 1 eq) and ethyl 2-oxoacetate (720.43 mg, 7.06 mmol, 2 eq) in MeOH (5 mL) was added CH₃COOH (635.68 mg, 10.59 mmol, 605.98 μL, 6 eq) and the mixture was stirred at 25° C. for 5 hrs. NaBH₃CN (443.47 mg, 7.06 mmol, 4 eq) was added and the mixture was stirred at 25° C. for 10 hrs. The mixture was diluted with water (5 mL) and extracted with DCM (5 mL*3). The combined organic layers were dried over Na₂SO₄, filtered and concentrated in vacuum to give a residue. The residue was purified by column chromatography (SiO₂, PE:EA=5:1 to DCM:MeOH=0:1). The eluted fractions were concentrated in vacuum to give 14-2 (650 mg, 1.58 mmol, 89.74% yield, 90% purity) as a light yellow oil. LCMS: (Method D) Rt=0.398 min, [M+H]⁺=370.4. LCMS: (Method D) Rt=0.279 min, [M+H]⁺=370.4. ¹H NMR (400 MHZ, DMSO-d₆) δ=4.13-4.03 (m, 2H), 3.91 (d, J=12.4 Hz, 2H), 3.47-3.33 (m, 4H), 2.97-2.96 (m, 4H), 2.71 (d, J=1.6 Hz, 4H), 1.91 (s, 3H), 1.67 (d, J=12.0 Hz, 2H), 1.39 (s, 9H), 1.21-1.17 (m, 3H), 1.02-1.00 (m, 2H).

### Step 2: Synthesis of 14-3

To a solution of 14-2 (600 mg, 1.62 mmol, 1 eq) in DCM (0.5 mL) was added HCl/dioxane (4 M, 1 mL, 2.46 eq). The mixture was stirred at 25° C. for 0.5 hr. The reaction mixture was concentrated in vacuum to give 14-3 (450 mg, crude, HCl salt) as a white solid which was used in the next step directly without further purification. LCMS (Method A): Rt=0.237 min, [M+H]⁺=270.3.

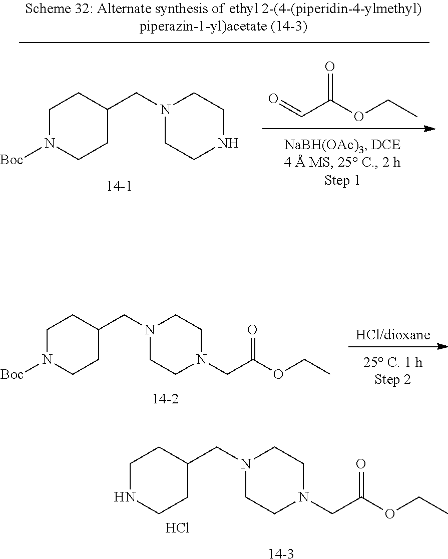

### Step 1 Synthesis of 14-2

To a solution of 14-1 (15 g, 52.93 mmol, 1 eq) in DCE (150 mL) was added 4 Å molecular sieves (1.5 g). The mixture was stirred at 25° C. for 0.25 h, followed by addition of ethyl 2-oxoacetate (16.21 g, 79.39 mmol, 1.5 eq) dropwise at 25° C. The mixture was stirred at 25° C. for 0.75 h then NaBH(OAc)₃ (28.04 g, 132.32 mmol, 2.5 eq) was added. The mixture was stirred at 25° C. for 1 h. The reaction mixture was slowly quenched with H₂O (200 mL) and then extracted with DCM (80 mL*3). The combined organic layers were dried over Na₂SO₄, filtered and concentrated under reduced pressure to give a residue. The residue was purified by flash silica gel chromatography (120 g SepaFlash silica flash column; eluent: 0-10% methanol/ethyl acetate at 100 mL/min), 14-2 (16 g, 43.30 mmol, 81.81% yield) was obtained as a yellow solid. ¹H NMR (400 MHZ, DMSO-d₆) δ 4.09-4.04 (m, 2H), 3.91-3.89 (d, J=11.6 Hz, 2H), 3.17 (s, 2H), 2.75-2.56 (m, 2H), 2.49-2.45 (m, 2H), 2.34 (s, 4H), 2.11-2.09 (d, J=6.8 Hz, 2H), 1.90 (s, 2H), 1.70-1.57 (m, 3H), 1.38 (s, 9H), 1.19-1.12 (m, 3H), 0.98-0.82 (m, 2H).

### Step 2: Synthesis of 14-3

To a solution of 14-2 (30 g, 81.19 mmol, 1 eq) in DCM (300 mL) was added HCl/dioxane (4 M, 300 mL, 14.78 eq). The mixture was stirred at 25° C. for 1 h. The reaction mixture was concentrated under reduced pressure to give a residue. The residue was used in the next step without further purification. 14-3 (24 g, 78.47 mmol, 96.65% yield, HCl salt) was obtained as a white solid. ¹H NMR (400 MHZ, METHANOL-d4) δ 4.42-4.28 (m, 4H), 3.99-3.74 (m, 7H), 3.66 (s, 1H), 3.46-3.43 (d, J=12.8 Hz, 2H), 3.34-3.32 (d, J=6.8 Hz, 2H), 3.08 (m, 2H), 2.34 (m, 1H), 2.23-2.20 (d, J=14.4 Hz, 2H), 1.67-1.52 (m, 2H), 1.34 (m, 3H).

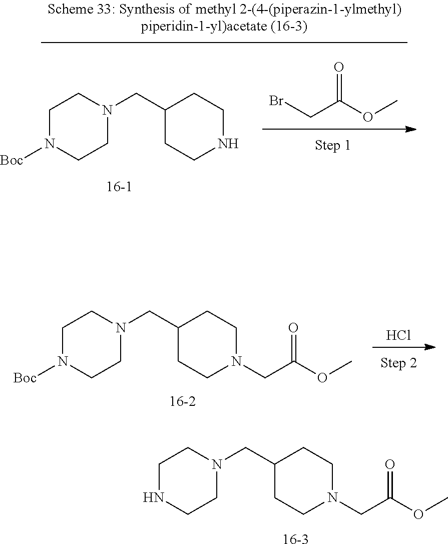

### Step 1: Synthesis of 16-2

To a solution of 16-1 (0.3 g, 1.06 mmol, 1 eq) and methyl 2-bromoacetate (178.12 mg, 1.16 mmol, 110.22 μL, 1.1 eq) in ACN (3 mL) was added DIEA (273.61 mg, 2.12 mmol, 368.75 μL, 2 eq) and the mixture was stirred at 40° C. for 1 hr. The mixture was then poured into water (1 mL) and extracted with DCM (1 mL*3) and the organic layers were dried over Na₂SO₄, filtered and concentrated to give 16-2 (0.37 g, 1.04 mmol, 98.33% yield) as a yellow solid. ¹H NMR (400 MHZ, CDCl₃) δ 3.72 (s, 3H), 3.43 (s, 4H), 3.23 (s, 2H), 2.94 (d, J=11.6 Hz, 2H), 2.36 (s, 4H), 2.19 (d, J=10.4 Hz, 3H), 2.01 (s, 2H), 1.76 (d, J=12.4 Hz, 2H), 1.46 (s, 9H), 1.-8-1.27 (m, 2H).

### Step 2: Synthesis of 16-3

A solution 16-2 (0.37 g, 1.04 mmol, 1 eq) in HCl/dioxane (4 M, 4 mL, 15.37 eq) was stirred at 25° C. for 0.5 hr. The mixture was concentrated to give 16-3 (0.3 g, 1.03 mmol, 98.77% yield, HCl salt) as a white solid. ¹H NMR (400 MHZ, methanol-d₄) δ 3.-8-3.84 (m, 3H), 3.71 (s, 4H), 3.66 (d, J=1.6 Hz, 2H), 3.-6-3.33 (m, 2H), 3.-0-3.15 (m, 4H), 2.-4-1.80 (m, 4H), 1.-6-1.67 (m, 1H), 1.38 (t, J=4.8 Hz, 4H).

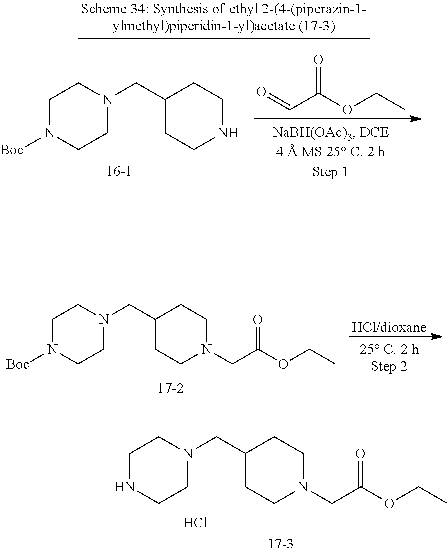

### Step 1: Synthesis of 17-2

To a solution of 16-1 (27 g, 95.27 mmol, 1 eq) in DCE (300 mL) was added 4 Å molecular sieves (2.7 g, 95.27 mmol, 1 eq). The mixture was stirred at 25° C. for 0.25 h, and then ethyl 2-oxoacetate (29.18 g, 142.90 mmol, 1.5 eq) was added dropwise at 25 C. Then the mixture was stirred at 25° C. for another 0.75 h. And NaBH(OAc)₃ (50.48 g, 238.17 mmol, 2.5 eq) was added. The mixture was stirred at 25° C. for 1 h. The reaction mixture was quenched slowly by addition of H₂O (600 mL), and then diluted with DCM (150 mL) and extracted with DCM (100 mL*3). The combined organic layers were dried over Na₂SO₄, filtered and concentrated under reduced pressure to give a residue. The residue was purified by flash silica gel chromatography (330 g SepaFlash silica flash column; eluent: 0-10% Methanol/Ethyl acetate at 100 mL/min), 17-2 (33 g, 89.31 mmol, 93.75% yield) was obtained as colorless oil. ¹H NMR (400 MHZ, CDCl₃) δ 4.19-4.14 (m, 2H), 3.51-3.38 (m, 5H), 3.27 (s, 2H), 3.03-3.00 (d, J=11.6 Hz, 2H), 2.43 (m, 4H), 2.32-2.24 (m, 4H), 2.01 (s, 2H), 1.75-1.72 (d, J=12.68 Hz, 2H), 1.43 (s, 9H), 1.30-1.26 (m, 3H).

### Step 2: Synthesis of 17-3

To a solution of 17-2 (32 g, 86.60 mmol, 1 eq) in DCM (300 mL) was added HCl/dioxane (4 M in dioxane, 21.65 mL, 1 eq). The mixture was stirred at 25° C. for 2 h. The reaction mixture was concentrated under reduced pressure to give a residue. The residue was used in the next step without further purification. 17-3 (28.3 g, 98.16% yield, HCl salt) was obtained as a white solid. ¹H NMR (400 MHZ, methanol-d₄) δ 4.33 (m, 2H), 4.19 (s, 2H), 3.75 (d, J=3.6 Hz, 8H), 3.62-3.37 (m, 2H), 3.32 (m, 2H), 3.23 (m, 2H), 2.46-2.22 (m, 3H), 1.82-1.65 (m, 2H), 1.34 (m, 3H).

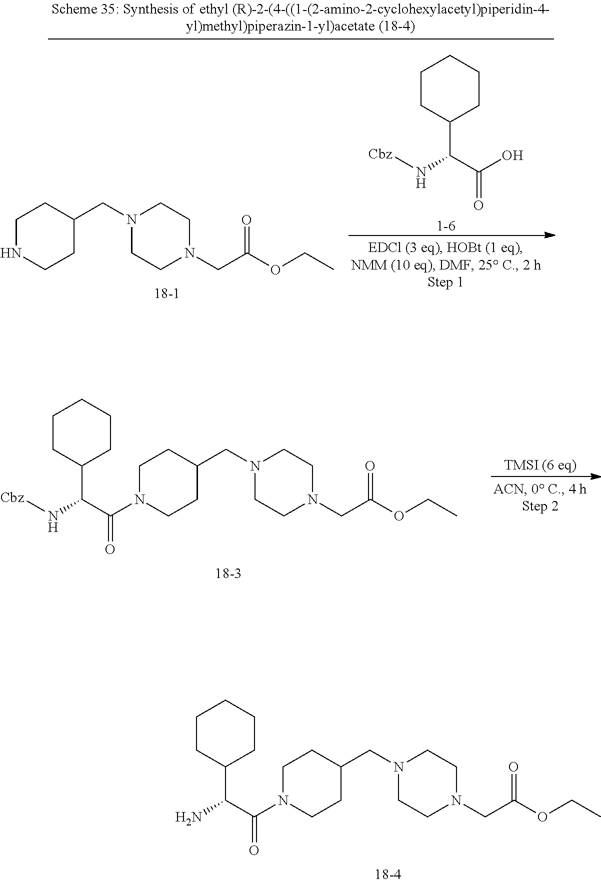

### Step 1: Synthesis of 18-3

To a solution of 18-1 (629.86 mg, 2.06 mmol, 1.2 eq, HCl salt) and 1-6 (500 mg, 1.72 mmol, 1 eq) in DMF (5 mL) was added EDCI (987.00 mg, 5.15 mmol, 3 eq), HOBt (231.90 mg, 1.72 mmol, 1 eq) and NMM (1.74 g, 17.16 mmol, 1.89 mL, 10 eq). The mixture was stirred at 25° C. for 2 h. The reaction mixture was quenched by addition H₂O (1 mL), diluted with EA (1 mL) and extracted with EA (2 mL*3). The combined organic layers were washed with brine (2 mL*3), dried over anhydrous Na₂SO₄, filtered and concentrated under reduced pressure to give a residue. The residue was used in the next step without further purification. 18-3 (800 mg, 1.42 mmol, 82.46% yield, 96.1% purity) was obtained as a yellow gum. LCMS (Method E): Rt=0.470 min, [M+H]⁺=543.4. SFC: Rt=1.184 min, ee>99%.

### Step 2: Synthesis of 18-4

To a solution of 18-3 (800 mg, 1.47 mmol, 1 eq) in ACN (24 mL) was added TMSI (1.77 g, 8.84 mmol, 1.20 mL, 6 eq). The mixture was stirred at 0° C. for 4 h. The reaction mixture was quenched by addition of H₂O (10 mL), diluted with EA (20 mL) and extracted with EA (20 mL*3). The combined organic layers were adjusted to pH 7-8 with a saturated solution of NaHCO₃ and extracted with CHCl₃:i-PrOH (3:1, 10 mL*3), dried over anhydrous Na₂SO₄, filtered and concentrated under reduced pressure to give a residue. The residue was used in the next step without further purification. 18-4 (550 mg, 1.29 mmol, 87.67% yield, 96.9% purity) was obtained as a yellow gum. LCMS (Method E): Rt=0.327 min, [M+H]⁺=409.2. SFC: Rt=2.420 min, ee=93.8%. ¹H NMR (400 MHZ, DMSO-d₆) δ 4.39 (d, J=12.0 Hz, 1H), 4.19-3.98 (m, 3H), 3.89 (d, J=13.2 Hz, 1H), 3.46-3.22 (m, 2H), 3.10 (d, J=6.0 Hz, 2H), 3.02-2.90 (m, 1H), 2.62-2.51 (m, 2H), 2.50-2.40 (m, 3H), 2.33 (s, 3H), 2.11-2.08 (m, 2H), 1.81-1.63 (m, 6H), 1.62-1.55 (m, 1H), 1.48 (d, J=8.4 Hz, 1H), 1.30 (d, J=1.6 Hz, 1H), 1.20-1.17 (m, 3H), 1.16-1.06 (m, 3H), 1.05-0.75 (m, 3H).

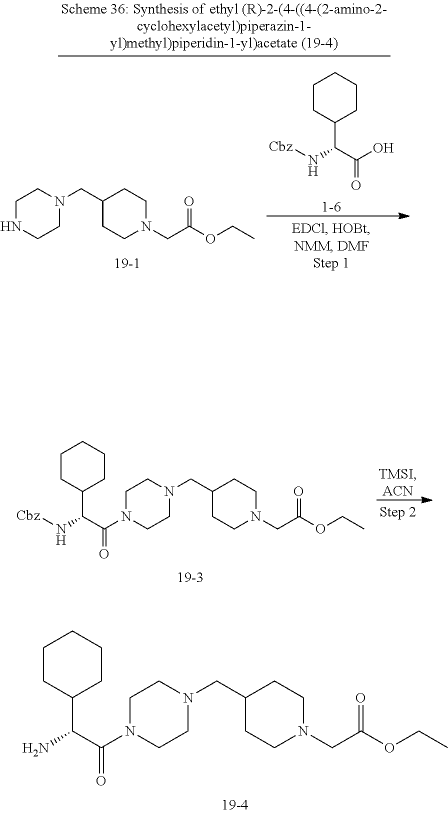

### Step 1: Synthesis of 19-3

To a solution of 19-1 (503.89 mg, 1.65 mmol, 1.2 eq) and 1-6 (400 mg, 1.37 mmol, 1 eq) in DMF (5 mL) was added EDCI (789.60 mg, 4.12 mmol, 3 eq), HOBt (185.52 mg, 1.37 mmol, 1 eq) and NMM (1.39 g, 13.73 mmol, 1.51 mL, 10 eq). The mixture was stirred at 25° C. for 1 hr. The mixture was poured into water (10 mL) and extracted with EA (10 mL*3). The combined organic layers were concentrated to give a residue. The residue was purified by flash silica gel chromatography (12 g SepaFlash silica flash column, eluent: 0-100% ethyl acetate/petroleum ether; gradient at 20 mL/min) and the eluent was concentrated to give 19-3 (300 mg, 542.06 μmol, 39.48% yield, 98.06% purity) as a yellow solid. LCMS (Method E): Rt=0.427 min, [M+H]⁺=543.3. SFC: Rt=2.199 min.

### Step 2: Synthesis of 19-4

To a solution of 19-3 (250 mg, 460.65 μmol, 1 eq) in ACN (3 mL) was added TMSI (553.04 mg, 2.76 mmol, 376.22 μL, 6 eq) at 0° C. The mixture was stirred at 0° C. for 4 hr. The reaction mixture was quenched by addition of H₂O (10 mL), diluted with EA (20 mL), and extracted with EA (20 mL*3). The combined organic layers were adjusted to pH 7-8 with a saturated solution of NaHCO₃, extracted with CHCl₃:i-PrOH (3:1, 10 mL*3), dried over anhydrous Na₂SO₄, filtered and concentrated under reduced pressure to give 19-4 (150 mg, 304.72 μmol, 66.15% yield, 83% purity) as a white solid. LCMS (Method E): Rt=0.317 min, [M+H]⁺=409.4.

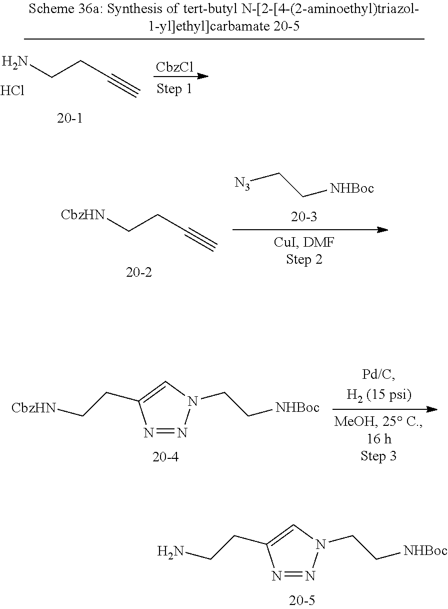

### Step 1: Synthesis of 20-2

To a solution of CbzCl (48.48 g, 284.18 mmol, 40.57 mL, 1 eq) in EtOH (480 mL) and H₂O (480 mL) was added NaHCO₃ (23.87 g, 284.18 mmol, 11.06 mL, 1 eq). Then 20-1 (30 g, 284.18 mmol, 1 eq, HCl) was added at 0° C. The mixture was stirred at 25° C. for 16 hr. The reaction mixture was quenched with water (500 mL). The mixture was extracted with EA (200 mL*3) and dried over anhydrous Na₂SO₄. The mixture was filtered and the filtrate was concentrated under vacuum. The residue was purified by flash silica gel chromatography (220 g SepaFlash silica flash column; eluent: 0-30% ethyl acetate/petroleum ether gradient at 120 mL/min) to give 20-2 (36 g, 132.85 mmol, 46.75% yield, 75% purity) as an off-white oil. LCMS (Method E): Rt=0.485 min, M+H=204.2. ¹H NMR (400 MHZ, CDCl₃) δ 7.38-7.35 (m, 5H), 5.11 (s, 2H), 3.42-3.27 (m, 2H), 2.47-2.34 (m, 2H), 2.11-1.99 (m, 1H).

### Step 2: Synthesis of 20-4

To a solution of 20-2 (36 g, 177.13 mmol, 1 eq) and 20-3 (32.98 g, 177.13 mmol, 1 eq) in DMF (300 mL) was added CuI (5.06 g, 26.57 mmol, 0.15 eq). The mixture was stirred at 80° C. for 16 hr. The reaction mixture was filtered through a pad of celite and the celite was washed with ethyl acetate. Then the mixture was diluted with H₂O (1500 mL) and extracted with EA (300 mL*3). The combined organic layers were washed with brine (200 mL*2), dried over anhydrous Na₂SO₄, filtered and concentrated under reduced pressure to give a residue. The residue was purified by flash silica gel chromatography (330 g SepaFlash silica flash column; eluent: 50-100% ethyl acetate/petroleum ether gradient at 150 mL/min), 20-4 (34 g, 82.94 mmol, 46.82% yield, 95% purity) was obtained as a white solid. LCMS (Method E): Rt=0.490 min, [M+H]⁺=390.2. ¹H NMR (400 MHZ, CDCl₃) δ 7.39-7.28 (m, 6H), 5.36 (s, 1H), 5.08 (s, 2H), 4.98 (s, 1H), 4.43-4.39 (m, 2H), 3.65-3.46 (m, 4H), 2.94-2.91 (m, 2H), 1.42 (s, 9H).

### Step 3: Synthesis of 20-5

To a solution of 20-4 (20 g, 51.35 mmol, 1 eq) in MeOH (100 mL) was added Pd/C (2.00 g, 1.88 mmol, 10% purity, 0.037 eq) under an N₂ atmosphere. The suspension was degassed and purged with H₂ 3 times. The mixture was stirred under H₂ (15 psi) at 25° C. for 16 hr. The reaction mixture was washed by MeOH 150 mL (50 mL*3) and then filtered to give a residue. The residue was used in the next step without further purification. 20-5 (13 g, 50.92 mmol, 99.15% yield) was obtained as an off-white oil. LCMS (Method E): Rt=0.313 min, [M+H]⁺=256.2. ¹H NMR (400 MHZ, CDCl₃) δ 7.39 (s, 1H), 5.31 (s, 1H), 4.43-4.39 (m, 2H), 3.60-3.55 (m, 2H), 3.07-2.95 (m, 2H), 2.84-2.79 (m, 2H), 1.39 (s, 9H).

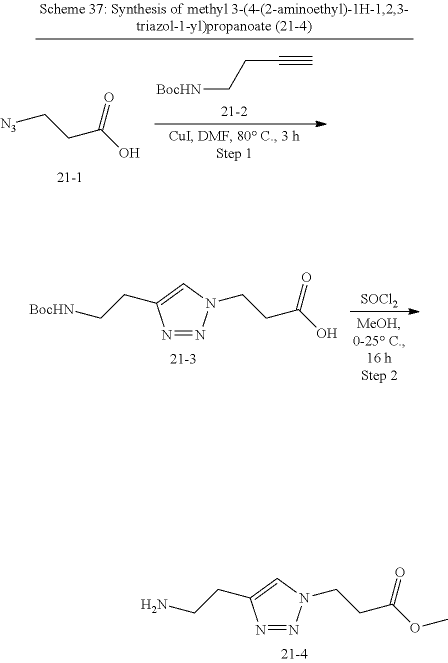

### Step 1: Synthesis of 21-3

To a solution of 21-1 (25 g, 217.22 mmol, 1 eq) in DMF (250 mL) was added CuI (4.14 g, 21.72 mmol, 0.1 eq) and 21-2 (36.76 g, 217.22 mmol, 1 eq). The mixture was stirred at 80° C. for 3 h. The reaction mixture was quenched by addition of water (500 mL) at 25° C., and then extracted with EA (100 mL*3). The organic phase was dried over Na₂SO₄, filtered and concentrated under reduced pressure to give a residue. The residue was purified by flash silica gel chromatography (330 g SepaFlash silica flash column; eluent: 0-100% ethyl acetate/petroleum ether gradient at 100 mL/min), 21-3 (42 g, 147.73 mmol, 68.01% yield) was obtained as yellow solid. LCMS (Method E): Rt=0.389 min, [M+H]⁺=285.2. ¹H NMR (400 MHz, CDCl₃) δ 9.76-8.99 (m, 1H), 7.52 (s, 1H), 5.22 (s, 1H), 4.59 (m, 2H), 3.46-3.23 (m, 2H), 2.93-2.89 (m, 2H), 2.83 (s, 2H), 1.37 (s, 9H).

### Step 2: Synthesis of 21-4

To a solution of 21-3 (40 g, 140.69 mmol, 1 eq) in MeOH (400 mL) was added SOCl₂ (20.09 g, 168.83 mmol, 12.26 mL, 1.2 eq) at 0° C. The mixture was stirred at 25° C. for 16 h. The mixture was concentrated under reduced pressure to give a residue. The crude product was used in the next step without further purification. 21-4 (32 g, 133.63 mmol, 94.98% yield, 98% purity, HCl salt) was obtained as a yellow solid. LCMS (Method E): Rt=0.17 min, [M+H]⁺=199.30. ¹H NMR (400 MHZ, METHANOL-d4) δ 8.57 (s, 1H), 4.88 (m, 2H), 3.71 (s, 3H), 3.39-3.37 (d, J=6.4 Hz, 2H), 3.32 (m, 2H), 3.15 (m, 2H).

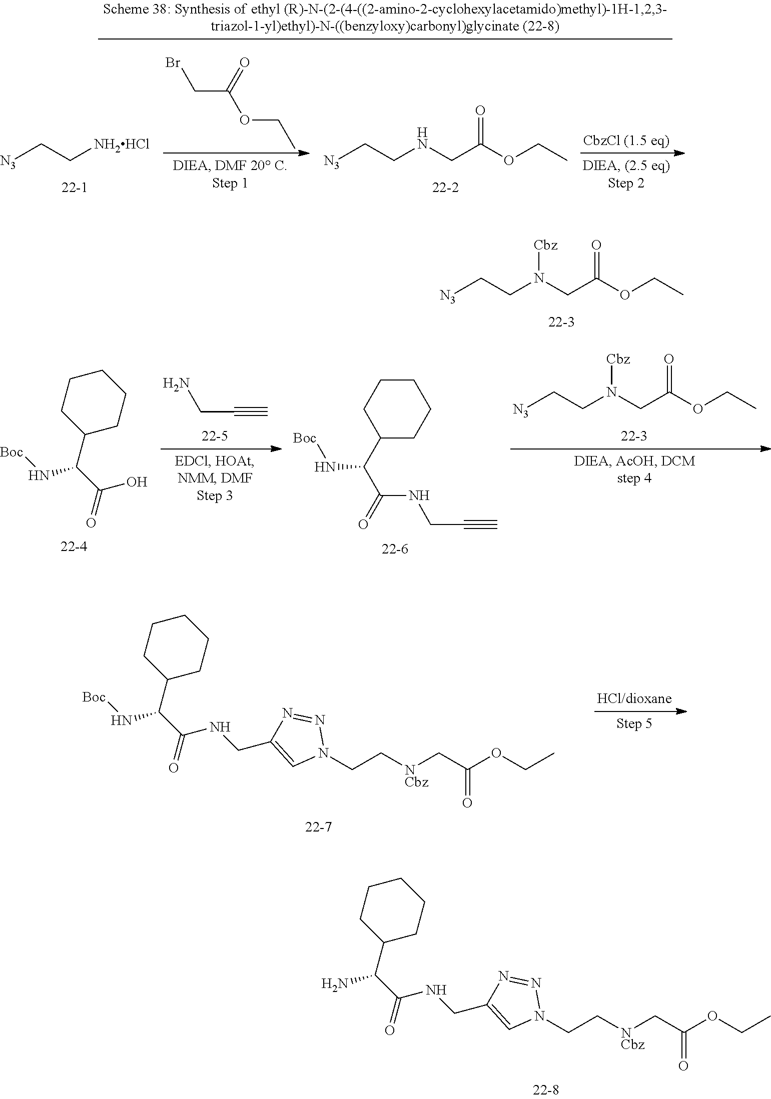

### Step 1: Synthesis of 22-2

To a solution of 22-1 (575 mg, 4.69 mmol, 1 eq) and ethyl 2-bromoacetate (470.11 mg, 2.82 mmol, 311.54 μL, 0.6 eq) in DMF (6 mL) was added TEA (569.71 mg, 5.63 mmol, 783.64 μL, 1.2 eq). The mixture was stirred at 25° C. for 4 hr. The reaction mixture was poured into water (5 ml) and extracted with EA (5 ml*2), and the organic layers were combined and concentrated in vacuum to afford the crude product. The residue was purified by flash silica gel chromatography to afford 22-2 (410 mg, 2.38 mmol, 50.7% yield) as a light-yellow oil. ¹H NMR (400 MHZ, CDCl₃) δ 4.28-4.02 (m, 2H), 3.49-3.34 (m, 4H), 2.95-2.86 (m, 1H), 2.83-2.80 (m, 1H), 1.31-1.22 (m, 3H).

### Step 2: Synthesis of 22-3

To a solution of 22-2 (390 mg, 2.27 mmol, 1 eq) in DCM (4 mL) was added CbzCl (579.58 mg, 3.40 mmol, 485.01 μL, 1.5 eq) and DIEA (731.83 mg, 5.66 mmol, 986.29 μL, 2.5 eq) at 0° C. The mixture was stirred at 25° C. for 1 hr. The reaction was poured into water (5 ml) and extracted with DCM (5 ml*3), and then the organic layer was separated and concentrated under vacuum to give the crude product. The residue was purified by flash silica gel chromatography to give 22-3 (500 mg, 1.52 mmol, 67.02% yield, 93% purity) as a yellow oil. LCMS (Method E): Rt=0.539 min, [M+Na)]⁺=329.1. ¹H NMR (400 MHZ, CDCl₃) δ 7.41-7.29 (m, 5H), 5.17 (d, J=18.4 Hz, 2H), 4.26-4.03 (m, 4H), 3.59-3.42 (m, 4H), 1.33-1.15 (m, 3H).

### Step 3: Synthesis of 22-6

To a solution of 22-4 (3.59 g, 13.95 mmol, 1 eq) and 22-5 (1.15 g, 20.93 mmol, 1.34 mL, 1.5 eq) in DMF (20 mL) was added EDCI (8.02 g, 41.85 mmol, 3 eq), HOAt (2.85 g, 20.93 mmol, 2.93 mL, 1.5 eq) and NMM (14.11 g. 139.51 mmol, 15.34 mL, 10 eq). The mixture was stirred at 25° C. for 2 hr. The reaction was poured into water (25 ml) and extracted with EA (25 ml*3), and then the organic layer was separated and concentrated under vacuum to give the crude product. The crude product was purified by recrystallization from DCM (50 mL) to give 22-6 (2.0 g, 5.98 mmol, 42.85% yield, 88% purity) as a white solid. LCMS (Method E): Rt=0.517 min, [M+H−56]⁺=239.2. LCMS (Method E): Rt=0.518 min, [M+H-56]⁺=239.2. ¹H NMR (400 MHZ, methanol-d₄) δ 4.13-3.99 (m, 1H), 3.93-3.84 (m, 2H), 2.59 (d, J=2.0 Hz, 1H), 1.75 (d, J=8.8 Hz, 3H), 1.69-1.58 (m, 3H), 1.44 (s, 9H), 1.31-0.97 (m, 6H).

### Step 4: Synthesis of 22-7

To a solution of 22-6 (100 mg, 339.69 μmol, 2 eq) and 22-3 (52.03 mg, 169.84 μmol, 1 eq) in DCM (1 mL) was added DIEA (15.37 mg, 118.89 μmol, 20.71 μL, 0.7 eq), CuI (6.47 mg, 33.97 μmol, 0.2 eq) and AcOH (7.14 mg, 118.89 μmol, 6.81 μL, 0.7 eq) at 0° C. The mixture was stirred at 25° C. for 12 hr. The reaction was poured into water (3 ml) and extracted with EA (3 ml*2), and then the organic layer was separated and concentrated under vacuum to give the crude product. The residue was purified by flash silica gel chromatography to give 22-7 (80 mg, 123.85 μmol, 72.92% yield, 93% purity) as a yellow oil. LCMS (Method E): Rt=0.576 min, [M+H]⁺=601.4. SFC: Rt=1.237 min: ee>99%.

### Step 5: Synthesis of 22-8

A solution of 22-7 (50 mg, 83.24 μmol, 1 eq) in HCl/dioxane (0.5 mL) was stirred at 25° C. for 1 hr. The reaction was concentrated under vacuum to give 22-8 (40 mg, 65.54 μmol, 78.74% yield. 88% purity, HCl salt), which was used in the next step without further purification. LCMS (Method E): Rt=0.442 min, [M+H]⁺=501.4.

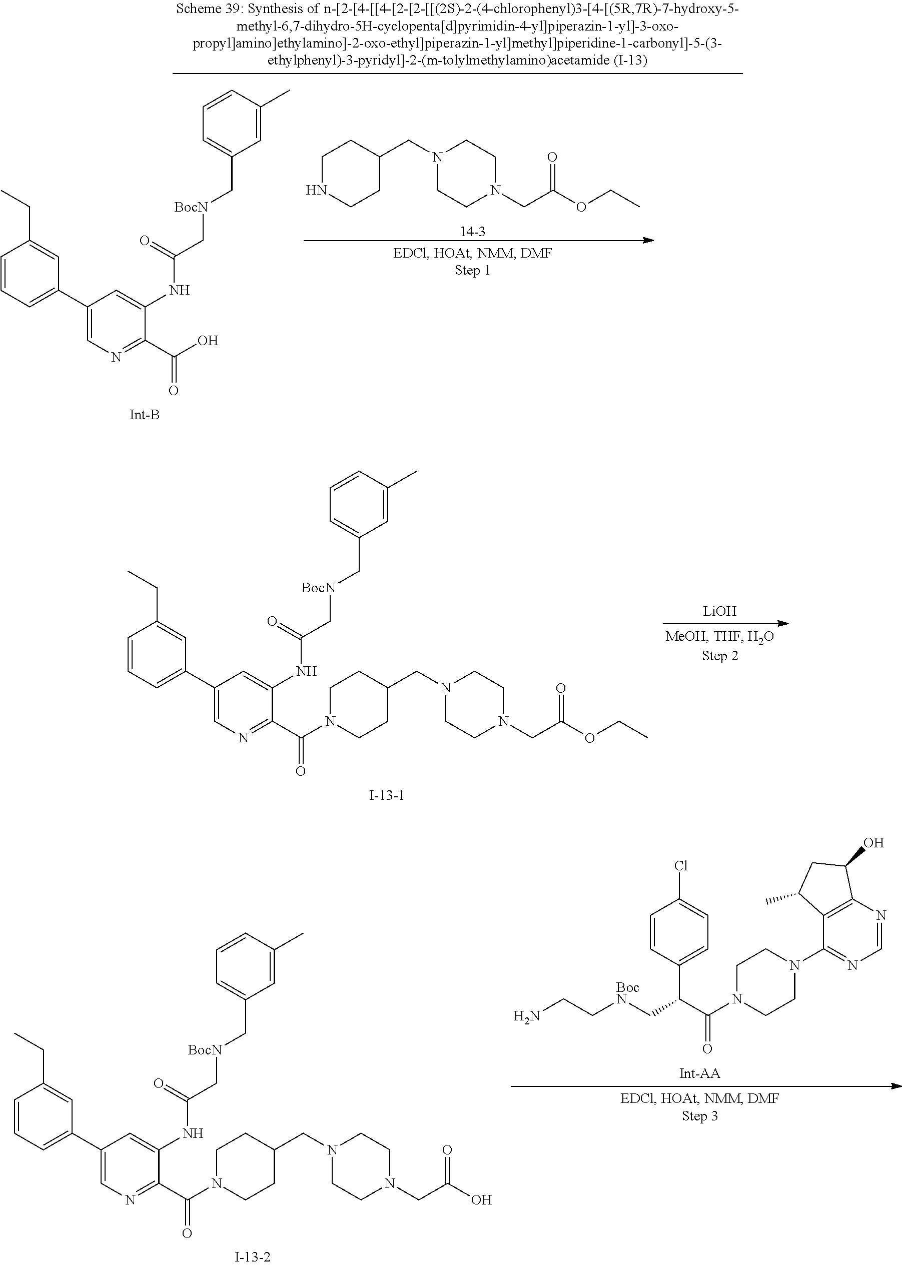

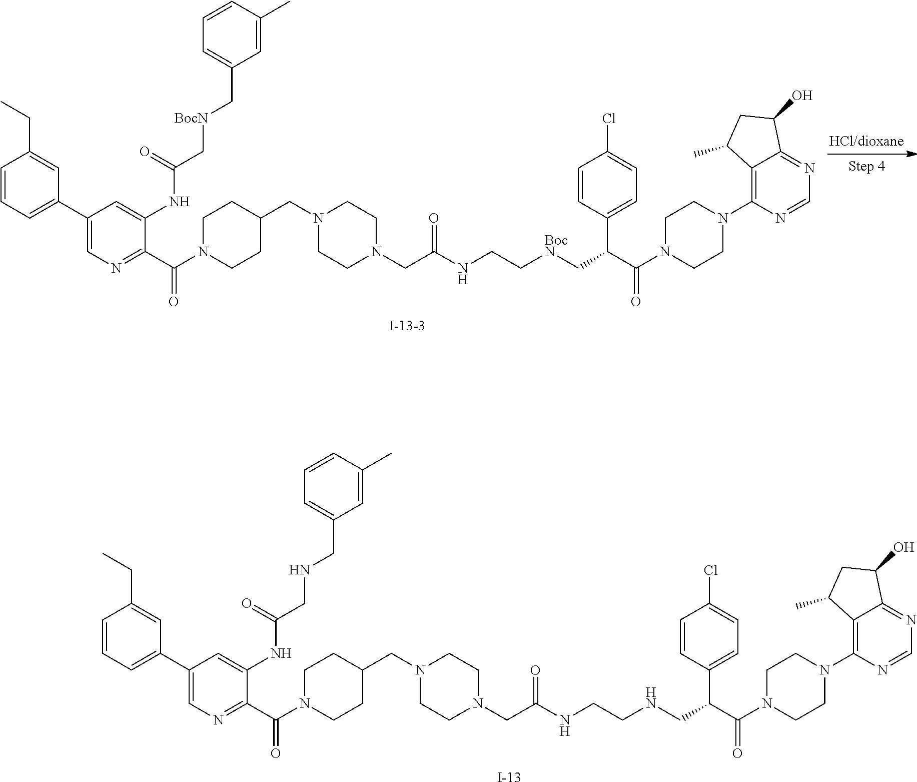

### Step 1: Synthesis of I-13-1

To a solution of 14-3 (100 mg, 198.57 μmol, 1 eq) and Int-B (78.95 mg, 258.15 μmol, 1.3 eq, HCl) in DMF (1 mL) was added EDCI (114.20 mg, 595.72 μmol, 3 eq), HOAt (27.03 mg, 198.57 μmol, 27.78 μL, 1 eq), and NMM (100.43 mg, 992.87 μmol, 109.16 μL, 5 eq). The mixture was stirred at 25° C. for 1 hr. The mixture was diluted with water (5 mL) and extracted with DCM (5 mL*3). The combined organic layers were dried over Na₂SO₄, filtered, and concentrated in vacuum to give a residue. The residue was purified by column chromatography (SiO₂, PE:EA=10:1 to PE:EA=0:1). I-13-1 (70 mg, 89.94 μmol, 45.29% yield, 97% purity) was obtained as a yellow oil. LCMS (Method D): Rt=0.456 min, [M+H]⁺=755.5.

### Step 2: Synthesis of I-13-2

To a solution of I-13-1 (50 mg, 66.23 μmol, 1 eq) in THF (0.2 mL), MeOH (0.2 mL), and H₂O (0.2 mL) was added LiOH·H₂O (8.34 mg, 198.69 μmol, 3 eq). The mixture was stirred at 25° C. for 1 hr. The reaction mixture was concentrated in vacuum to remove THF and MeOH to give a residue. The residue was diluted with water (5 mL) and extracted with DCM (5 mL*3), and then the aqueous phase was acidified with hydrochloric acid (1 M) to pH=5-6. The aqueous phase was then extracted with DCM (5 mL*3). The combined organic layers were dried over Na₂SO₄, filtered, and concentrated in vacuum to give I-13-2 (45 mg, 61.91 μmol, 93.47% yield) as a yellow gum. LCMS (Method D): Rt=0.445 min, [M+H]⁺=727.4.

### Step 3: Synthesis of I-13-3

To a solution of I-13-2 (30 mg, 53.66 μmol, 1 eq) and Int-AA (39.00 mg, 53.66 μmol, 1 eq) in DMF (0.5 mL) was added EDCI (30.86 mg, 160.97 μmol, 3 eq), HOAt (7.30 mg, 53.66 μmol, 7.51 μL, 1 eq), and NMM (27.14 mg, 268.29 μmol, 29.50 μL, 5 eq). The mixture was stirred at 25° C. for 0.5 hr. The mixture was diluted with water (5 mL) and extracted with DCM (5 mL*3). The combined organic layers were dried over Na₂SO₄, filtered, and concentrated in vacuum to give a residue. The residue was purified by column chromatography (SiO₂, PE:EA=10:1 to DCM:MeOH=0:1), and then the organic liquid was concentrated in vacuum to give I-13-3 (30 mg, 22.24 μmol, 41.45% yield, 94% purity) as a white oil. LCMS (Method D): Rt=0.447 min, [M+2H]²⁺=637.9. LCMS (Method D): Rt=0.454 min, [M+H]⁺=1267.8.

### Step 4: Synthesis of I-13

To a solution of I-13-3 (30 mg, 23.66 μmol, 1 eq) in DCM (0.3 mL) was added HCl/dioxane (4 M, 0.3 mL, 50.72 eq). The mixture was stirred at 25° C. for 20 min. The reaction mixture was concentrated in vacuum to give a residue. The residue was purified by prep-HPLC (column: Welch Xtimate C18 150 mm*25 mm*5 um; mobile phase: [water (FA):ACN]; gradient: 5%-35% ACN over 10 min) and lyophilized. I-13 (10.27 mg, 9.25 μmol, 39.10% yield, 99.469% purity, FA salt) was obtained as a white solid. LCMS (Method D): Rt=0.319 min [M+H]⁺=1067.8. SFC: Rt=0.747 min. ¹H NMR (400 MHZ, methanol-d₄) δ=8.62 (d, J=1.6 Hz, 1H), 8.57 (d, J=1.6 Hz, 1H), 8.45-8.34 (m, 3H), 7.55-7.41 (m, 5H), 7.38-7.29 (m, 4H), 7.28-7.24 (m, 2H), 7.13 (d, J=4.0 Hz, 1H), 4.97 (t, J=6.4 Hz, 1H), 4.92-4.88 (m, 1H), 4.67 (d, J=13.6 Hz, 1H), 4.45-4.43 (m, 1H), 3.97-3.87 (m, 3H), 3.84-3.70 (m, 3H), 3.63-3.57 (m, 3H), 3.56-3.45 (m, 6H), 3.45-3.39 (m, 1H), 3.28-3.18 (m, 3H), 3.15 (s, 5H), 2.92-2.85 (m, 1H), 2.78 (s, 3H), 2.75 (d, J=7.6 Hz, 2H), 2.71 (d, J=11.2 Hz, 4H), 2.50 (d, J=6.4 Hz, 2H), 2.35 (s, 3H), 2.17-1.97 (m, 3H), 1.90 (d, J=13.6 Hz, 1H), 1.77 (d, J=13.6 Hz, 1H), 1.42-1.34 (m, 1H), 1.32-1.24 (m, 4H), 1.10 (d, J=6.4 Hz, 3H).

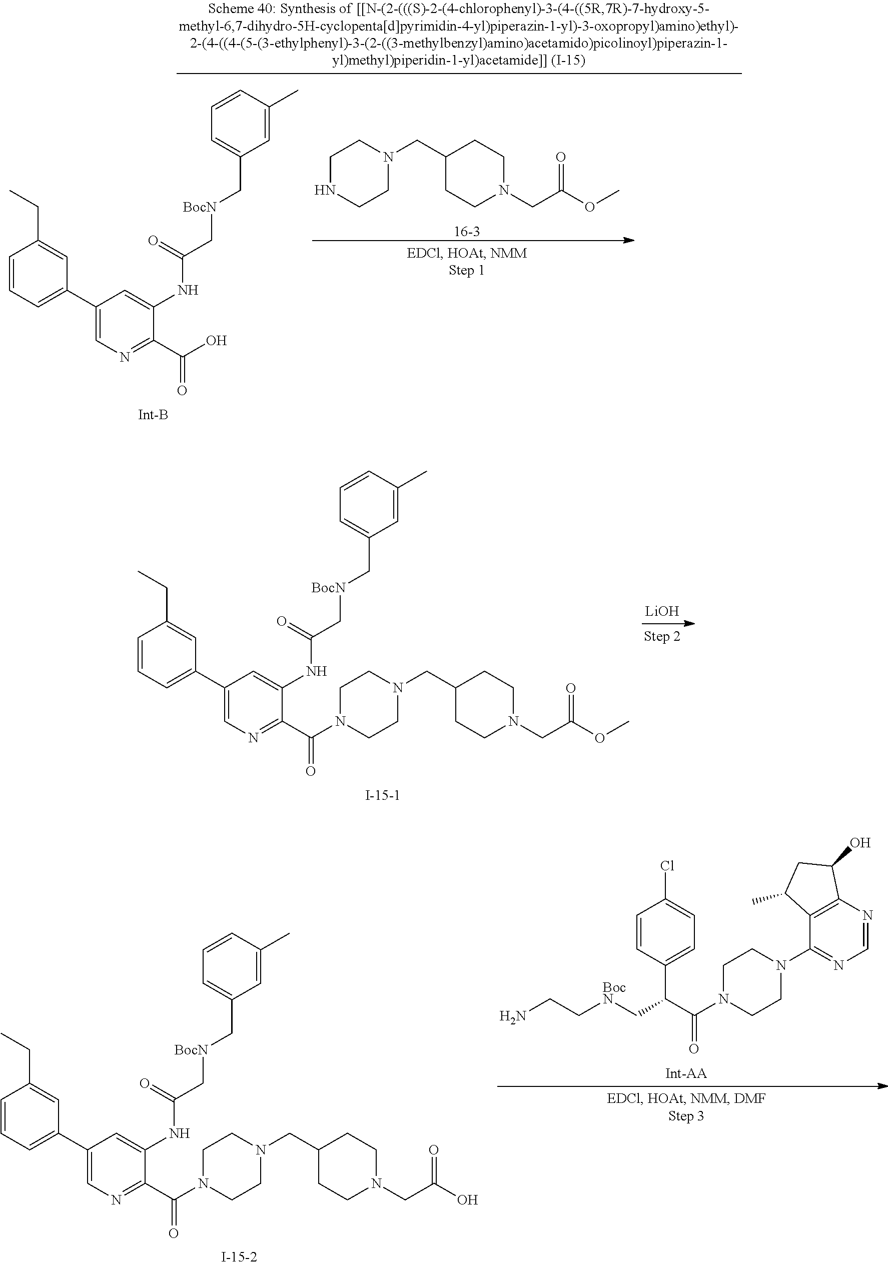

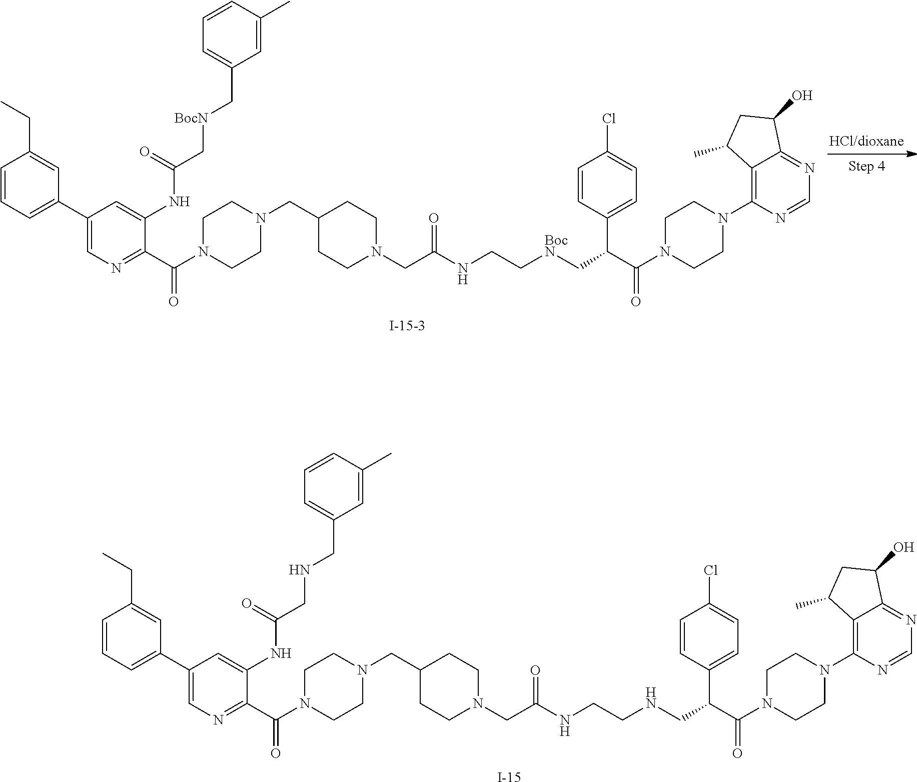

### Step 1: Synthesis of I-15-1

To a solution of 16-3 (0.3 g. 1.03 mmol. 1 eq. HCl salt) and Int-B (517.71 mg, 1.03 mmol. 1 eq) in DMF (3 mL) was added EDCI (394.16 mg, 2.06 mmol, 2 eq). HOAt (69.96 mg, 514.02 μmol, 71.91 μL. 0.5 eq) and NMM (519.92 mg, 5.14 mmol, 565.13 μL, 5 eq). The mixture was stirred at 25° C. for 1 hr. The mixture was poured into water (5 mL) and extracted with EA (5 mL*3), and the organic layers were dried over Na₂SO₄, filtered and concentrated to give a residue. The residue was purified by column chromatography (SiO₂, DCM/MeOH=100/1 to 1/100) and concentrated to give I-15-1 (0.28 g, 375.71 μmol, 36.55% yield, 99.42% purity) as a yellow solid. LCMS (Method D): Rt=0.389 min, [M+H]⁺=741.6.

### Step 2: Synthesis of I-15-2

To a solution of I-15-1 (0.28 g, 377.90 μmol, 1 eq) in THF (1 mL), MeOH (1 mL) and H₂O (1 mL) was added LiOH·H₂O (79.28 mg, 1.89 mmol, 5 eq). The mixture was stirred at 25° C. for 1 hr. The mixture was concentrated and adjusted to pH=3 with 0.5 M aq. HCl, extracted with EA (2 mL*3), and the organic layers were dried over Na₂SO₄, filtered and concentrated to give I-15-2 (0.27 g, crude) as a yellow solid.

### Step 3: Synthesis of I-15-3

To a solution of I-15-2 (0.04 g, 55.03 μmol, 1.5 eq) and Int-AA (20.51 mg, 36.69 μmol, 1 eq) in DMF (0.5 mL) was added EDCI (14.07 mg, 73.37 μmol, 2 eq), HOAt (2.50 mg, 18.34 μmol, 2.57 μL, 0.5 eq) and NMM (18.55 mg, 183.43 μmol, 20.17 μL, 5 eq). The mixture was stirred at 25° C. for 1 hr. The mixture was filtered to give a residue. The residue was purified by reversed-phase HPLC (0.1% FA). The eluent was concentrated, extracted with EA (5 mL*3), and the organic layers were dried over Na₂SO₄, filtered and concentrated to give I-15-3 (0.02 g, 15.77 μmol, 43.00% yield) as a white solid. LCMS (Method F): Rt=0.838 min, [M+H]⁺=1267.7. LCMS (Method D): Rt=0.376 min, [M+2H]²⁺=634.6.

### Step 4: Synthesis of I-15

A solution of I-15-3 (0.02 g, 15.77 μmol, 1 eq) in HCl/dioxane (4 M, 0.2 mL, 50.72 eq) was stirred at 25° C. for 0.5 hr. The mixture was concentrated to give a residue. The residue was purified by preparative HPLC (column: Phenomenex luna C18 150 mm*25 mm*10 um; mobile phase: [water (FA):ACN]; gradient: 5%-35% ACN over 10 min) and the eluent was lyophilized to give I-15 (6 mg, 5.62 μmol, 35.63% yield, 100% purity) as a white solid. LCMS (Method D): Rt=0.313 min, [M+H]⁺=1067.7. SFC: Rt=0.893 min. ¹H NMR (400 MHZ, methanol-d₄) δ=8.66 (s, 1H), 8.49-8.35 (m, 2H), 8.26-8.19 (m, 1H), 7.54-7.39 (m, 7H), 7.37-7.28 (m, 4H), 7.20 (s, 1H), 5.00-4.94 (m, 2H), 4.15-4.05 (m, 2H), 3.91 (s, 2H), 3.88-3.76 (m, 6H), 3.73-3.53 (m, 12H), 3.43-3.37 (m, 2H), 3.26-3.22 (m, 4H), 3.09-2.98 (m, 2H), 2.78-2.72 (m, 3H), 2.67-2.62 (m, 3H), 2.42 (d, J=6.4 Hz, 1H), 2.37 (s, 3H), 2.13-1.98 (m, 4H), 1.96-1.86 (m, 1H), 1.70-1.56 (m, 2H), 1.29 (t, J=7.6 Hz, 3H), 1.10 (d, J=6.8 Hz, 3H).

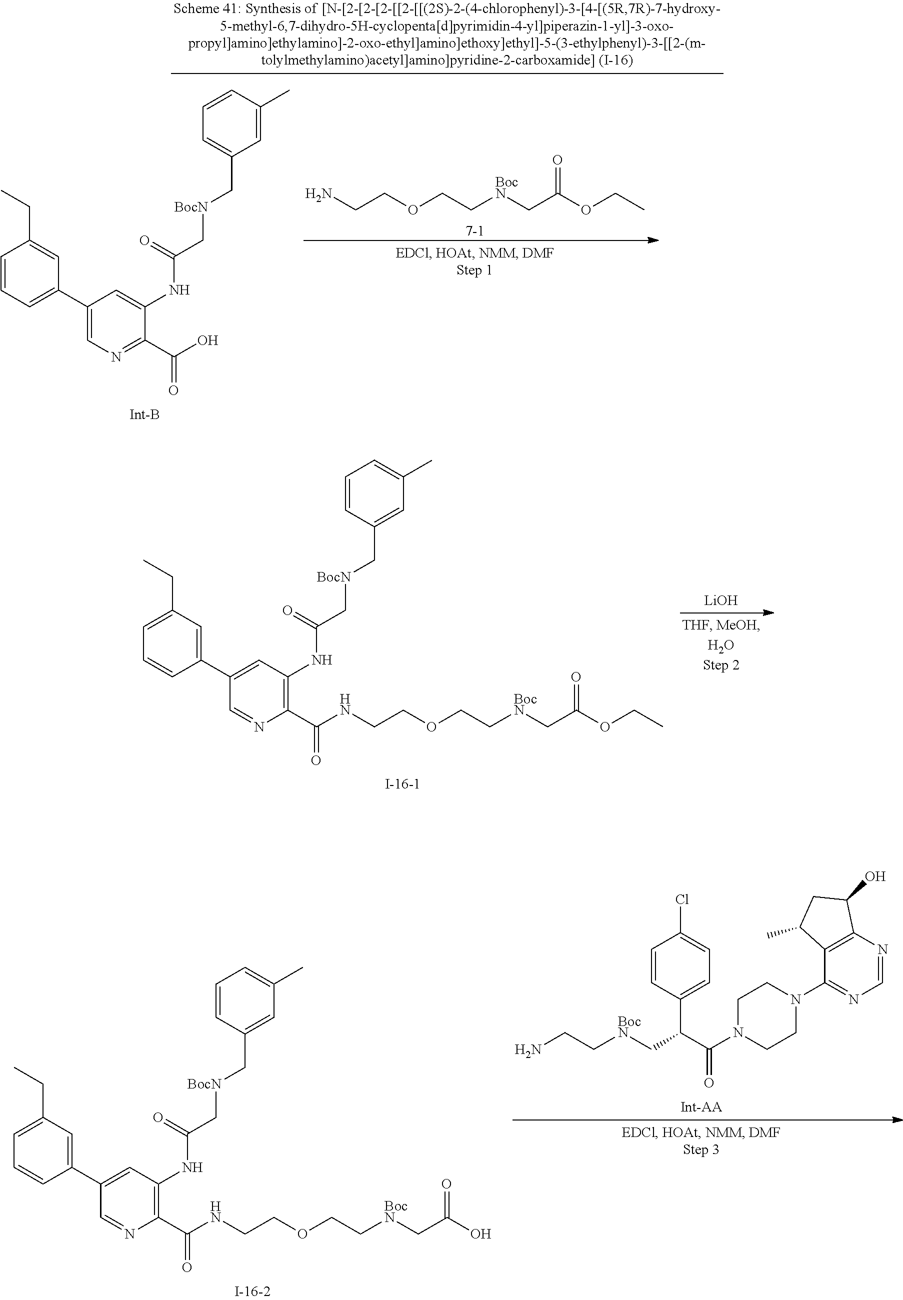

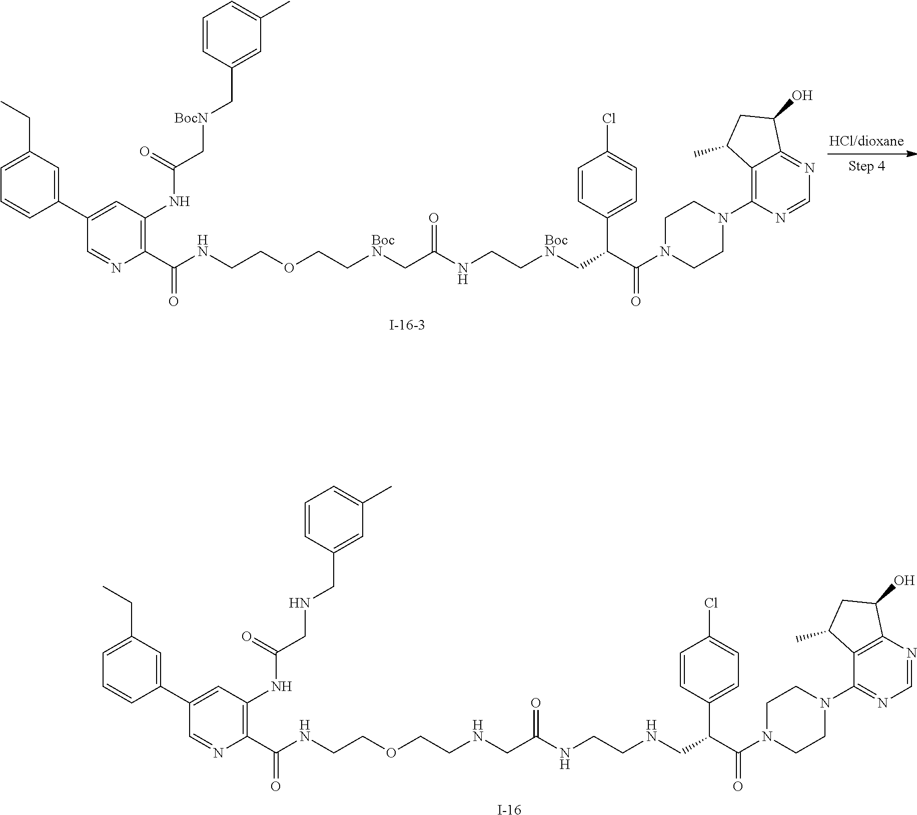

### Step 1: Synthesis of I-16-1

To a solution of Int-B (190.78 mg, 378.85 μmol, 1.1 eq) in DMF (1 mL) was added HOAt (93.75 mg, 688.81 μmol, 96.36 μL, 2 eq), EDCI (330.12 mg, 1.72 mmol, 5 eq), NMM (348.36 mg, 3.44 mmol, 378.65 μL, 10 eq) and 7-1 (100 mg, 344.41 μmol, 1 eq). The mixture was stirred at 25° C. for 2 hr. The reaction mixture was poured into water (10 mL) and extracted with EA (10 mL*3). The combined organic layers were dried over Na₂SO₄, filtered and concentrated under reduced pressure to give a residue. The residue was purified by flash silica gel chromatography (12 g SepaFlash silica flash column; eluent: 0-100% ethyl acetate/petroleum ether; gradient at 50 mL/min) to give I-16-1 (115 mg, 148.21 μmol, 43.03% yield) as a white oil. LCMS (Method D): Rt=0.646 min, [M+H]⁺=776.8.

### Step 2: Synthesis of I-16-2

To a solution of I-16-1 (120 mg, 154.65 μmol, 1 eq) in THF (0.4 mL), MeOH (0.4 mL) and H₂O (0.4 mL) was added LiOH·H₂O (19.47 mg, 463.96 μmol, 3 eq). The mixture was stirred at 25° C. for 1 hr. The pH of the mixture was adjusted to 5-6 with sat. aq. citric acid and extracted with EA 20 mL (5 mL*4). The combined organic layers were dried over Na₂SO₄, filtered and concentrated under reduced pressure to give I-16-2 (105 mg, crude) as a white solid. The crude product was used into the next step without further purification. LCMS (Method D): Rt=0.603 min, [M+H]⁺=748.4.

### Step 3: Synthesis of I-16-3

To a solution of 3-2 (44.14 mg, 59.02 μmol, 1.1 eq) in DMF (0.3 mL) was added HOAt (14.61 mg, 107.32 μmol, 15.01 μL, 2 eq), EDCI (51.43 mg, 268.29 μmol, 5 eq) and NMM (54.27 mg, 536.58 μmol, 58.99 μL, 10 eq), followed by Int-AA (30 mg, 53.66 μmol, 1 eq). The mixture was stirred at 25° C. for 1 hr. The reaction mixture was poured into water (5 mL) and extracted with EA 15 mL (5 mL*3). The combined organic layers were dried over Na₂SO₄, filtered and concentrated under reduced pressure to give I-16-3 (55 mg, 42.67 μmol, 79.52% yield) as a yellow oil. The residue was used into the next step without further purification. LCMS (Method D): Rt=0.557 min, [M+H]⁺=1288.7

### Step 4: Synthesis of I-16

To a solution of I-16-3 (50 mg, 38.79 μmol, 1 eq) in DCM (0.25 mL) was added HCl/dioxane (4 M, 0.25 mL). The mixture was stirred at 25° C. for 1 hr. The reaction mixture was concentrated under reduced pressure to give a residue. The residue was purified by prep-HPLC (column: Phenomenex luna C18 150 mm*25 mm*10 um; mobile phase: [water (FA):ACN]; gradient: 15%-45% ACN over 8 min). The eluent was lyophilized to give I-16 (20 mg, 18.57 μmol, 47.87% yield, 96.06% purity, FA salt) as a white solid. LCMS (Method D): Rt=0.337 min, [M+H]⁺=988.7. ¹H NMR (400 MHZ, methanol-d₄) δ 9.28 (d, J=2.0 Hz, 1H), 8.58 (d, J=2.0 Hz, 1H), 8.41-8.38 (m, 2H), 7.52-7.47 (m, 2H), 7.44-7.39 (m, 3H), 7.34-7.30 (m, 4H), 7.27-7.20 (m, 2H), 7.10 (d, J=7.2 Hz, 1H), 4.96 (t, J=6.8 Hz, 2H), 4.43-4.40 (m, 1H), 3.96 (s, 2H), 3.87-3.82 (m, 1H), 3.79-3.69 (m, 8H), 3.67 (d, J=2.0 Hz, 5H), 3.53-3.43 (m, 7H), 3.18-3.12 (m, 3H), 3.09-3.05 (m, 2H), 2.73 (q, J=7.6 Hz, 2H), 2.33 (s, 3H), 2.14-2.01 (m, 2H), 1.28 (t, J=7.6 Hz, 3H), 1.10-1.05 (m, 3H).

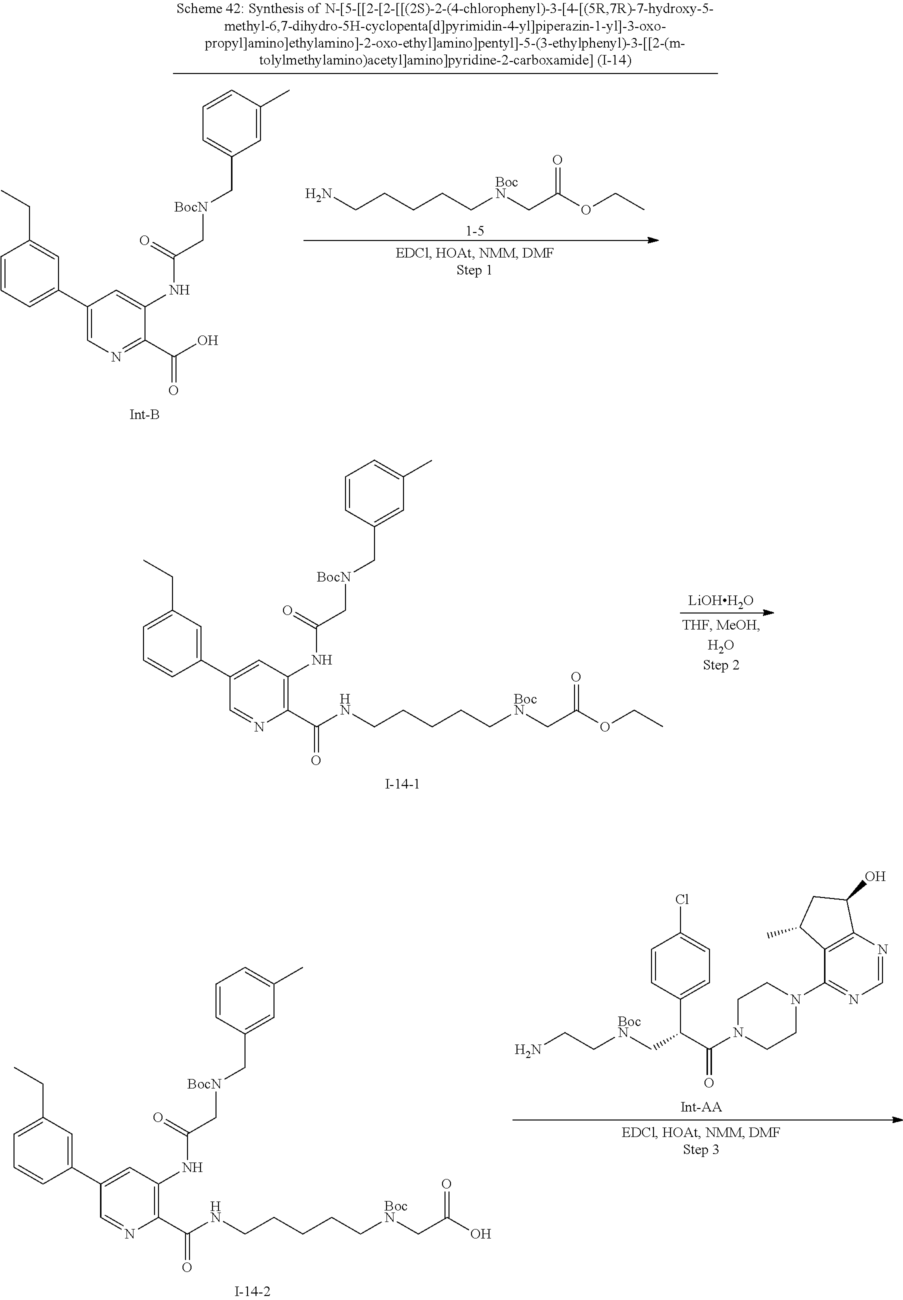

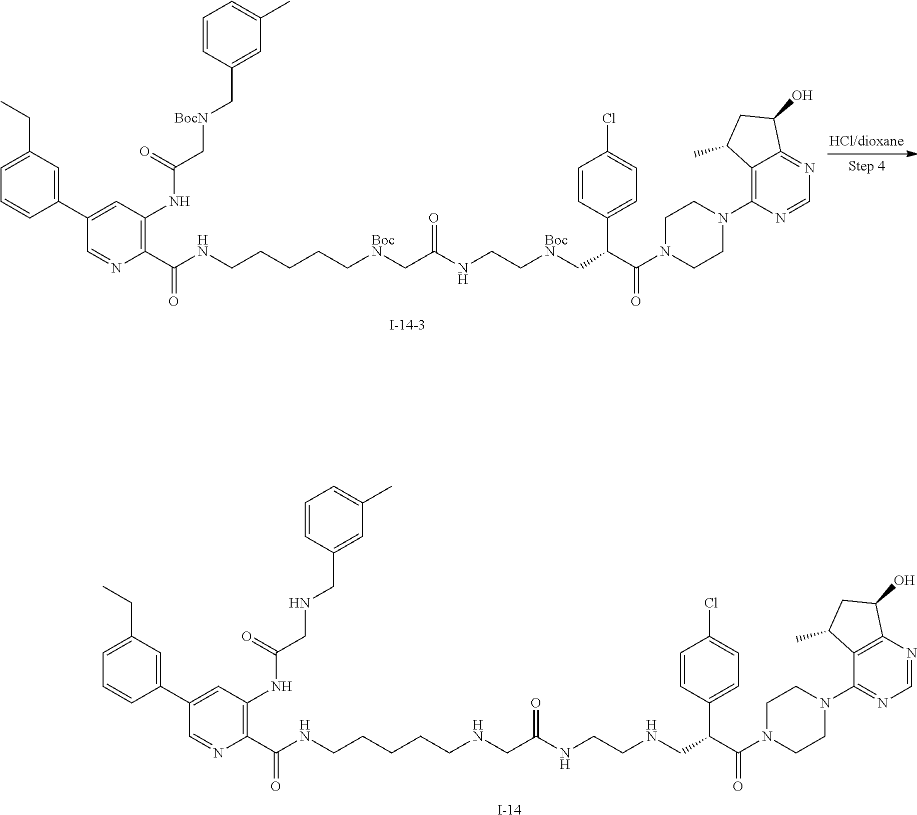

### Step 1: Synthesis of I-14-1

To a solution of Int-B in DMF (2 mL) was added EDCI (398.85 mg, 2.08 mmol, 3 eq). HOAt (94.40 mg, 693.52 μmol, 97.02 μL, 1 eq) and NMM (350.74 mg, 3.47 mmol, 381.24 μL, 5 eq) at 25° C. for 15 min. Then 1-5 (200 mg, 693.52 μmol, 1 eq) was added at 25° C. The resulting mixture was stirred at 25° C. for 45 min. The mixture was washed with water (5 mL) and extracted with EA (8 mL*3). The combined organic layers dried over sodium sulfate, filtered and concentrated under reduced pressure to give a residue. The residue was purified by column chromatography (SiO₂, PE/EA=1/0 to 0/1, Rf=0.4, PE:EA=3:1) to give I-14-1 (150 mg, 172.49 μmol, 24.87% yield, 89% purity) as a yellow oil. LCMS (Method D): Rt=0.604 min, [M+H]⁺=774.4. ¹H NMR (400 MHZ, methanol-d₄) δ=9.24 (d, J=7.6 Hz, 1H), 8.57 (s, 1H), 7.53-7.48 (m, 2H), 7.43-7.42 (m, 1H), 7.31 (d, J=7.6 Hz, 1H), 7.21-7.16 (m, 1H), 7.12 (d, J=10.8 Hz, 2H), 7.05 (d, J=7.6 Hz, 1H), 4.62 (s, 2H), 4.21-4.13 (m, 2H), 4.07-3.95 (m, 2H), 3.93 (d, J=10.6 Hz, 2H), 3.44-3.31 (m, 2H), 2.74-2.71 (m, 2H), 2.28 (s, 3H), 1.68 (d, J=6.8 Hz, 2H), 1.63-1.58 (m, 2H), 1.54 (s, 4H), 1.50-1.34 (m, 18H), 1.27-1.21 (m, 6H).

### Step 2: Synthesis of I-14-2

To a solution of I-14-1 (150 mg, 193.81 μmol, 1 eq) in THF (0.5 mL), MeOH (0.5 mL) and H₂O (0.5 mL) was added LiOH·H₂O (16.27 mg, 387.62 μmol, 2 eq). The mixture was stirred at 25° C. for 1 hr. The pH was acidified to 3-4 with 1N HCl, and the mixture was diluted with water (3 mL) and extracted with EA (5 mL*3). The combined organic layers dried over sodium sulfate, filtered and concentrated under reduced pressure to give I-14-2 (110 mg, 131.25 μmol, 67.72% yield, 89% purity) as a colorless gum. LCMS (Method D): Rt=0.695 min, [M+H]⁺=746.5.

### Step 3: Synthesis of I-14-3

To a solution of I-14-2 (40.02 mg, 53.66 μmol, 1 eq) and Int.-A (30 mg, 53.66 μmol, 1 eq) in DMF (0.5 mL) was added EDCI (30.86 mg, 160.97 μmol, 3 eq), NMM (27.14 mg, 268.29 μmol, 29.50 μL, 5 eq) and HOAt (7.30 mg, 53.66 μmol, 7.51 μL, 1 eq). The mixture was stirred at 25° C. for 1 hr. The mixture was washed with water (3 mL) and extracted with EA (5 mL*3). The combined organic layers were dried over sodium sulfate, filtered and concentrated under reduced pressure to give a residue. The residue was purified by column chromatography (SiO₂, EA/MeOH=1/0 to 0/1, Rf=0.3, PE:EA=0:1) to give I-14-3 (40 mg, 30.15 μmol, 56.19% yield, 97% purity) as a colorless oil. LCMS (Method D): Rt=0.573 min, [M+H]⁺=1287.1.

### Step 4: Synthesis of I-14

To a solution of I-14-3 (40 mg, 31.08 μmol, 1 eq) in dioxane (0.5 mL) was added HCl/dioxane (4 M, 0.5 mL, 64.35 eq). The mixture was stirred at 25° C. for 0.5 hr. The reaction mixture was concentrated under vacuum. The crude product was added to water and lyophilized to give I-14 (24 mg, 22.69 μmol, 73.01% yield, 96.73% purity, HCl) as a yellow solid. LCMS (Method D): Rt=0.355 min, [M+2H]²⁺=494.0. LCMS (Method D): Rt=0.373 min, [M+H]⁺=986.5. SFC: Rt=2.021 min. ¹H NMR (400 MHz, methanol-d₄) δ 9.22 (d, J=2.0 Hz, 1H), 8.63 (d, J=2.0 Hz, 1H), 8.57 (s, 1H), 7.54-7.47 (m, 2H), 7.47-7.38 (m, 6H), 7.37-7.32 (m, 3H), 7.32-7.27 (m, 1H), 5.31-5.29 (m, 1H), 4.76-4.72 (m, 1H), 4.31 (s, 2H), 4.19 (s, 2H), 4.07-3.93 (m, 3H), 3.91 (s, 2H), 3.85-3.53 (m, 9H), 3.47-3.45 (m, 2H), 3.34 (d, J=4.4 Hz, 1H), 3.27-3.26 (m, 2H), 3.17-3.09 (m, 2H), 2.75-2.74 (m, 2H), 2.40 (s, 3H), 2.32-2.26 (m, 1H), 2.22-2.14 (m, 1H), 1.83-1.81 (m, 2H), 1.78-1.69 (m, 2H), 1.59-1.48 (m, 2H), 1.30-1.27 (m, 3H), 1.23-1.13 (m, 3H).

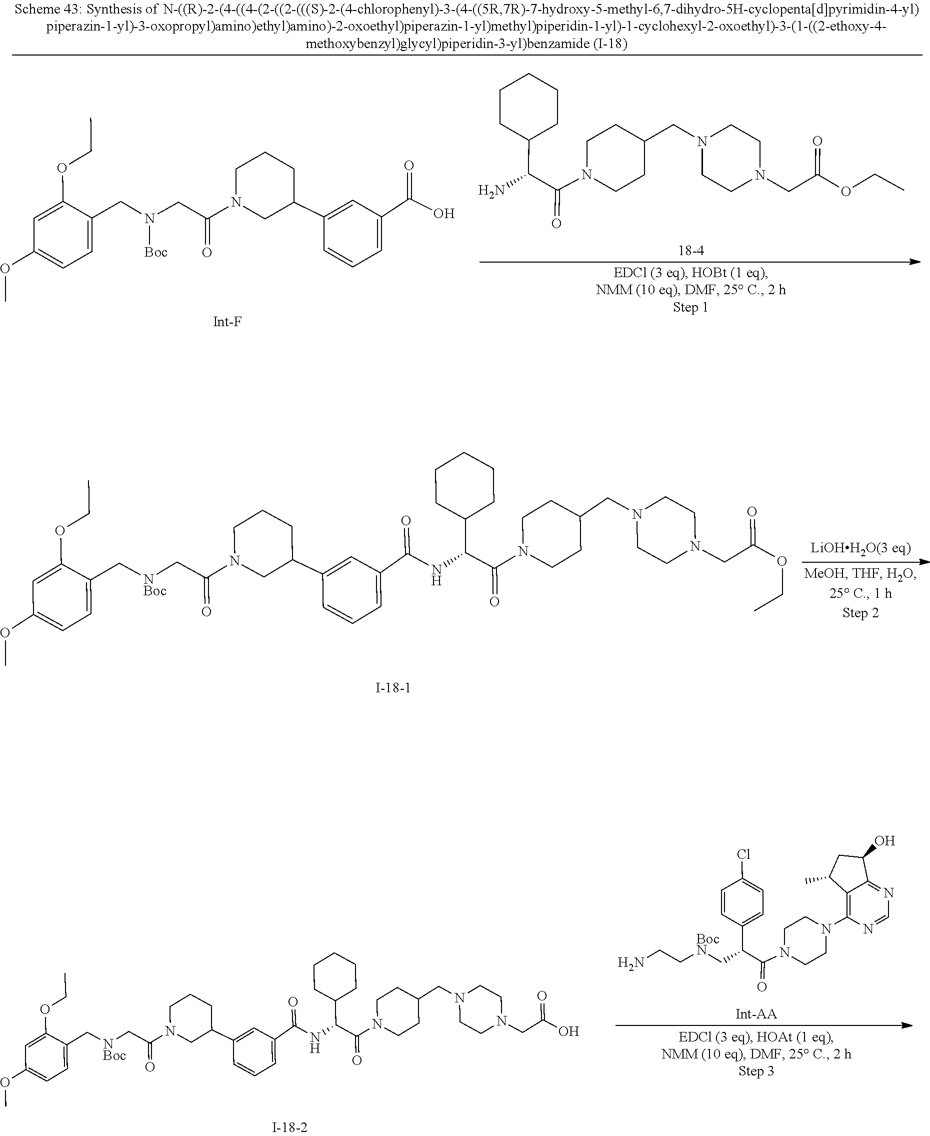 [SMILES: CCOc1cc(OC)ccc1CN(CC(=O)N1CCCC(c2cccc(C(=O)O)c2)C1)C(=O)OC(C)(C)C ; CCOC(=O)CN1CCN(CC2CCN(C(=O)[C@H](N)C3CCCCC3)CC2)CC1 ; CCOC(=O)CN1CCN(CC2CCN(C(=O)[C@H](NC(=O)c3cccc(C4CCCN(C(=O)CN(Cc5ccc(OC)cc5OCC)C(=O)OC(C)(C)C)C4)c3)C3CCCCC3)CC2)CC1 ; CCOc1cc(OC)ccc1CN(CC(=O)N1CCCC(c2cccc(C(=O)N[C@@H](C(=O)N3CCC(CN4CCN(CC(=O)O)CC4)CC3)C3CCCCC3)c2)C1)C(=O)OC(C)(C)C ; C[C@@H]1C[C@@H](O)c2ncnc(N3CCN(C(=O)[C@H](CN(CCN)C(=O)OC(C)(C)C)c4ccc(Cl)cc4)CC3)c21]

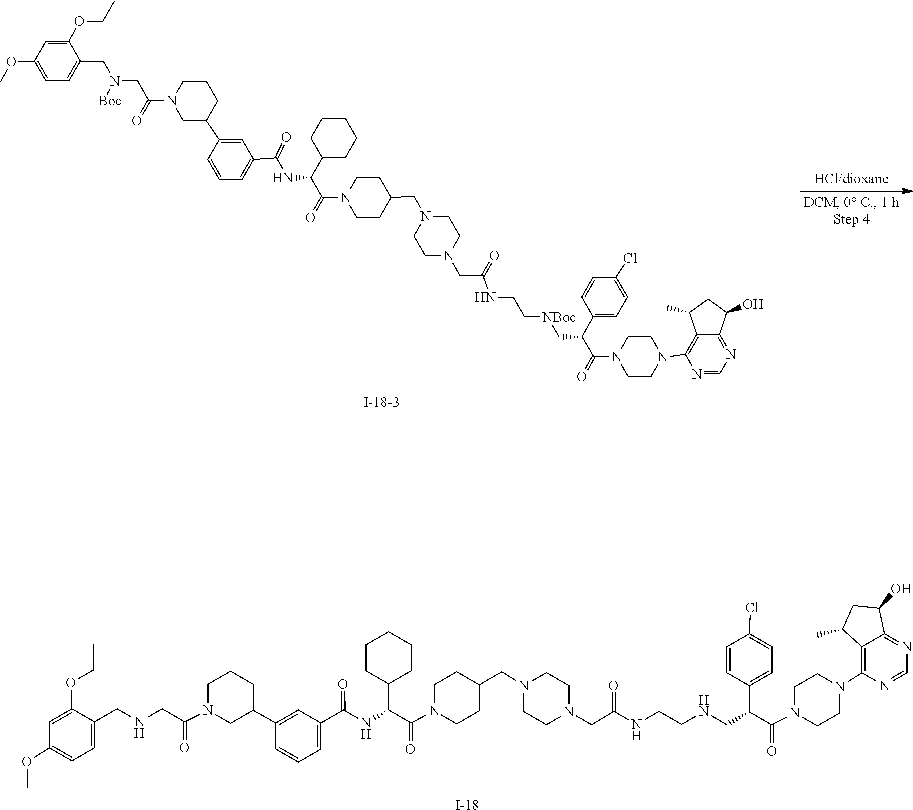

### Step 1: Synthesis of I-18-1

To a solution of 18-4 (111.72 mg, 273.44 μmol, 1.2 eq) and Int-F (120 mg, 227.87 μmol, 1 eq) in DMF (1.5 mL) was added EDCI (131.05 mg, 683.61 μmol, 3 eq), HOBt (30.79 mg, 227.87 μmol, 1 eq) and NMM (230.48 mg, 2.28 mmol, 250.52 μL, 10 eq). The mixture was stirred at 25° C. for 2 h. The reaction mixture was quenched by addition H₂O (2 mL), diluted with EA (2 mL) and extracted with EA (4 mL*3). The combined organic layers were washed with brine (4 mL*3), dried over anhydrous Na₂SO₄, filtered and concentrated under reduced pressure to give a residue. The residue was used in next step without further purification. I-18-1 (160 mg, 150.02 μmol, 65.84% yield, 86% purity) was obtained as a white solid. LCMS (Method E): Rt=0.564 min, (M+H)+=917.8.

### Step 2: Synthesis of I-18-2

To a solution of I-18-1 (160 mg, 174.45 μmol, 1 eq) in THF (0.8 mL), H₂O (0.5 mL) and MeOH (0.8 mL) was added LiOH·H₂O (21.96 mg, 523.34 μmol, 3 eq). The mixture was stirred at 25° C. for 1 h. The reaction mixture was concentrated under reduced pressure to give a residue. The residue was used in the next step without further purification. I-18-2 (150 mg, 143.40 μmol, 82.20% yield, 85% purity) was obtained as a white solid. LCMS (Method E): Rt=0.535 min, [M+H]⁺=889.6.

### Step 3: Synthesis of I-18-3

To a solution of 5-2 (80 mg, 89.98 μmol, 1 eq) and Int-AA (40.24 mg, 71.98 μmol, 0.8 eq) in DMF (1 mL) was added EDCI (51.75 mg, 269.93 μmol, 3 eq), HOAt (18.37 mg, 134.96 μmol, 18.88 μL, 1.5 eq) and NMM (91.01 mg, 899.76 μmol, 98.92 μL, 10 eq). The mixture was stirred at 25° C. for 1 h. The reaction mixture was quenched by addition of H₂O (1 mL), diluted with EA (1 mL) and extracted with EA (2 mL*3). The combined organic layers were washed with brine (2 mL*3), dried over anhydrous Na₂SO₄, filtered and concentrated under reduced pressure to give a residue. The residue was purified by reversed phase HPLC, then concentrated to remove the organic solvents and lyophilized to give the product. I-18-3 (20 mg, 13.98 μmol, 15.54% yield, 99% purity) was obtained as a white solid. LCMS (Method B): Rt=0.758 min, (M+H)+=1431.0.

### Step 4: Synthesis of I-18

To a solution of I-18-3 (15 mg, 10.49 μmol, 1 eq) in DCM (0.5 mL) was added HCl/dioxane (4M, 300.00 μL, 114.42 eq). The mixture was stirred at 0° C. for 1 h. The reaction mixture was filtered and concentrated under reduced pressure to give a residue. The residue was purified by prep-HPLC (column: Waters Xbridge 150 mm*25 mm*5 um; mobile phase: [water (NH₄HCO₃):ACN]; gradient: 48%-78% ACN over 9 min) and then concentrated and lyophilized to give I-18 (2.4 mg, 1.85 μmol, 17.67% yield, 95% purity) as a white solid. LCMS (Method E): Rt=0.443 min, [M+H]⁺=1229.8. SFC: Rt=5.829 min, Rt=10.238 min. ¹H NMR (400 MHZ, methanol-d₄) δ=8.41 (d, J=1.6 Hz, 1H), 7.79-7.66 (m, 2H), 7.49-7.29 (m, 6H), 7.18-7.08 (m, 1H), 6.57-6.41 (m, 2H), 5.01-4.92 (m, 3H), 4.63-4.43 (m, 3H), 4.36-4.25 (m, 1H), 4.22-4.15 (m, 1H), 4.00-3.99 (m, 1H), 4.12-3.99 (m, 1H), 3.91-3.69 (m, 9H), 3.66-3.41 (m, 8H), 3.03-2.96 (m, 2H), 2.86-2.62 (m, 7H), 2.61-2.39 (m, 8H), 2.29-1.98 (m, 6H), 1.97-1.64 (m, 12H), 1.49-1.38 (m, 4H), 1.35-1.22 (m, 4H), 1.17-1.04 (m, 6H).

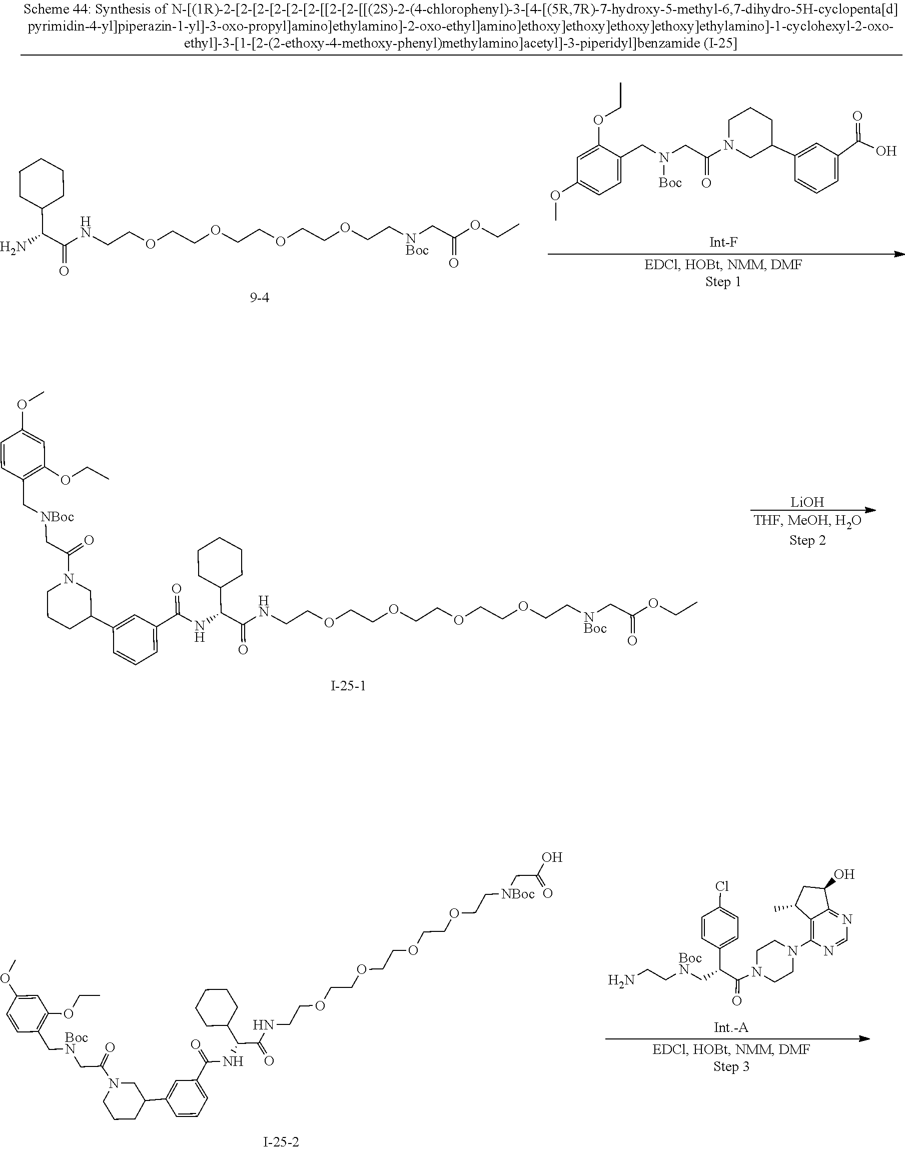 [SMILES: CCOC(=O)CN(CCOCCOCCOCCOCCNC(=O)[C@H](N)C1CCCCC1)C(=O)OC(C)(C)C ; CCOc1cc(OC)ccc1CN(CC(=O)N1CCCC(c2cccc(C(=O)O)c2)C1)C(=O)OC(C)(C)C ; CCOC(=O)CN(CCOCCOCCOCCOCCNC(=O)[C@H](NC(=O)c1cccc(C2CCCN(C(=O)CN(Cc3ccc(OC)cc3OCC)C(=O)OC(C)(C)C)C2)c1)C1CCCCC1)C(=O)OC(C)(C)C ; "LiOH" ; CCOc1cc(OC)ccc1CN(CC(=O)N1CCCC(c2cccc(C(=O)N[C@@H](C(=O)NCCOCCOCCOCCOCCN(CC(=O)O)C(=O)OC(C)(C)C)C3CCCCC3)c2)C1)C(=O)OC(C)(C)C ; C[C@@H]1C[C@@H](O)c2ncnc(N3CCN(C(=O)[C@H](CN(CCN)C(=O)OC(C)(C)C)c4ccc(Cl)cc4)CC3)c21]

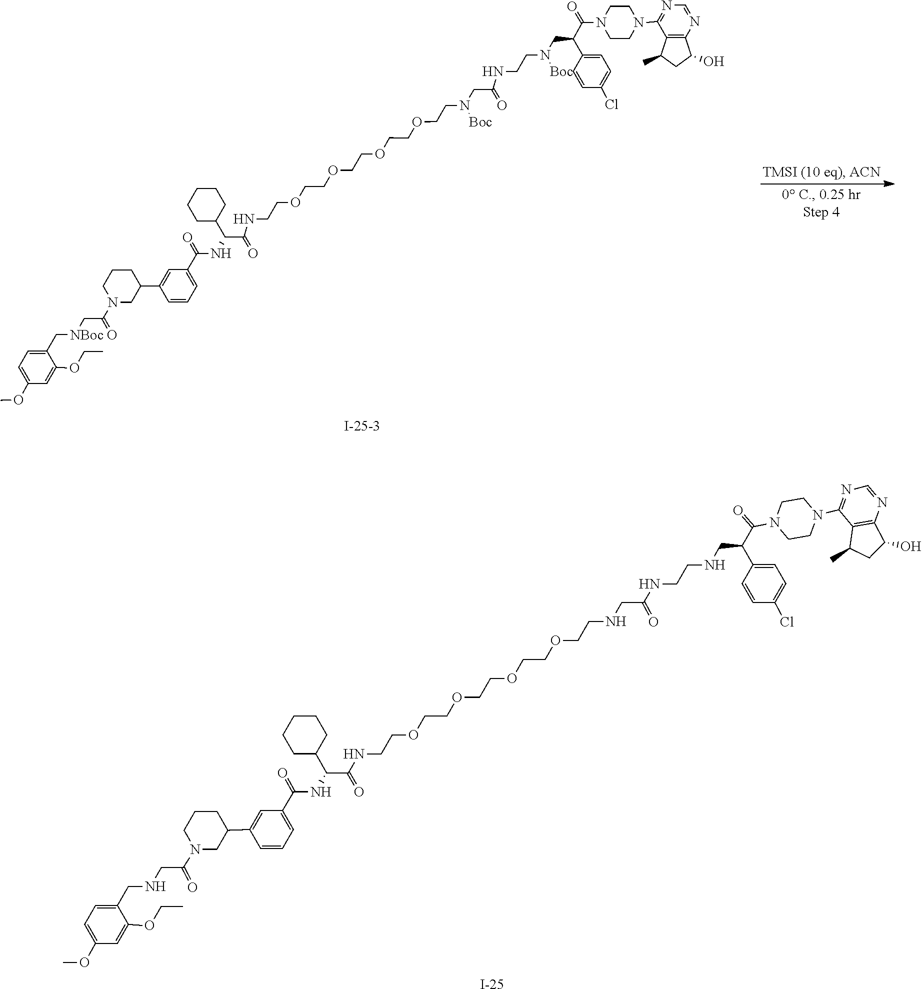

### Step 1: Synthesis of I-25-1

To a solution of Int-F (92.82 mg, 176.25 μmol, 0.9 eq) in DCM (2 mL) and 9-4 was added EDCI (75.08 mg, 391.66 μmol, 2 eq), NMM (99.04 mg, 979.16 μmol, 107.65 μL, 5 eq) and HOBt (13.23 mg, 97.92 μmol, 0.5 eq). The mixture was stirred at 25° C. for 1 hr. The reaction mixture was concentrated under reduced pressure to give a residue. The residue was purified by flash silica gel chromatography (12 g SepaFlash silica flash column; eluent: 50-80% ethyl acetate/petroleum ether; gradient at 20 mL/min). I-25-1 (120 mg, 112.12 μmol, 57.25% yield) was obtained as off-white oil. LCMS (Method E): Rt=0.667 min, [M+H-Boc]⁺=970.8. SFC: Rt=1.895 min.

### Step 2: Synthesis of I-25-2

To a solution of I-25-1 (110 mg, 102.77 μmol, 1 eq) in THF (1 mL) was added LiOH·H₂O (8.63 mg, 205.55 μmol, 2 eq) in MeOH (1 mL) and H₂O (1 mL). The mixture was stirred at 25° C. for 1 hr. The reaction mixture was quenched with water (5 mL). The mixture was extracted with EA (5 mL*3) and dried over anhydrous Na₂SO₄. The mixture filtered and the filtrate was concentrated under vacuum. The crude product was used in the next step without further purification. I-25-2 (110 mg, crude) was obtained as off-white oil. LCMS (Method E): Rt=0.625 min, [M+2H−100]⁺=942.7.

### Step 3: Synthesis of I-25-3

To a solution of I-25-2 (74.57 mg, 71.54 μmol, 1 eq) and Int-AA (40 mg, 71.54 μmol, 1 eq) in DMF (2 mL) was added EDCI (27.43 mg, 143.09 μmol, 2 eq), NMM (36.18 mg, 357.72 μmol, 39.33 μL, 5 eq) and HOBt (4.83 mg, 35.77 μmol, 0.5 eq). The mixture was stirred at 25° C. for 1 hr. The reaction mixture was quenched with water (5 mL). The mixture was extracted with EA (5 mL*3) and dried over anhydrous Na₂SO₄. The mixture was filtered and the filtrate was concentrated under vacuum. The residue was used in the next step without further purification. I-25-3 (80 mg, 50.53 μmol, 70.62% yield) was obtained as a white solid. LCMS (Method E): Rt=0.616 min, [M+2H]²⁺=792.4. LCMS (Method E): Rt=0.608 min, [M+H]⁺=1583.7. SFC: Rt=1.566 min.

### Step 4: Synthesis of I-25

To a solution of I-25-3 (70 mg, 44.21 μmol, 1 eq) in ACN (2 mL) was added TMSI (88.46 mg, 442.10 μmol, 60.18 μL, 10 eq) at 0° C. The mixture was stirred at 0° C. for 0.25 hr. The reaction mixture was quenched with water (5 mL). The mixture was extracted with EA (5 mL*3) and dried over anhydrous Na₂SO₄. The mixture filtered and the filtrate was concentrated under vacuum. The residue was purified by prep-HPLC (column: Phenomenex luna C18 150 mm*25 mm*10 um; mobile phase: [water (FA):ACN]; gradient: 5%-35% ACN over 10 min), followed by lyophilization. I-25 (20.52 mg, 15.44 μmol, 34.92% yield, 99% purity, FA salt) was obtained as a white solid. LCMS (Method E): Rt=0.443 min, [M+H]⁺=1282.9. SFC: Rt=3.612, 7.177 min. ¹H NMR (400 MHz, methanol-d₄) δ 7.77-7.71 (m, 2H), 7.49-7.29 (m, 7H), 7.26-7.21 (m, 1H), 6.61-6.51 (m, 2H), 4.99-4.94 (m, 1H), 4.51 (d, J=16.0 Hz, 1H), 4.43-4.02 (m, 7H), 3.93-3.69 (m, 10H), 3.65-3.58 (m, 16H), 3.57-3.43 (m, 10H), 3.37 (d, J=4.8 Hz, 4H), 2.83 (s, 6H), 2.17-2.09 (m, 1H), 2.08-2.01 (m, 2H), 1.91-1.64 (m, 10H), 1.48-1.40 (m, 3H), 1.36-1.05 (m, 10H).

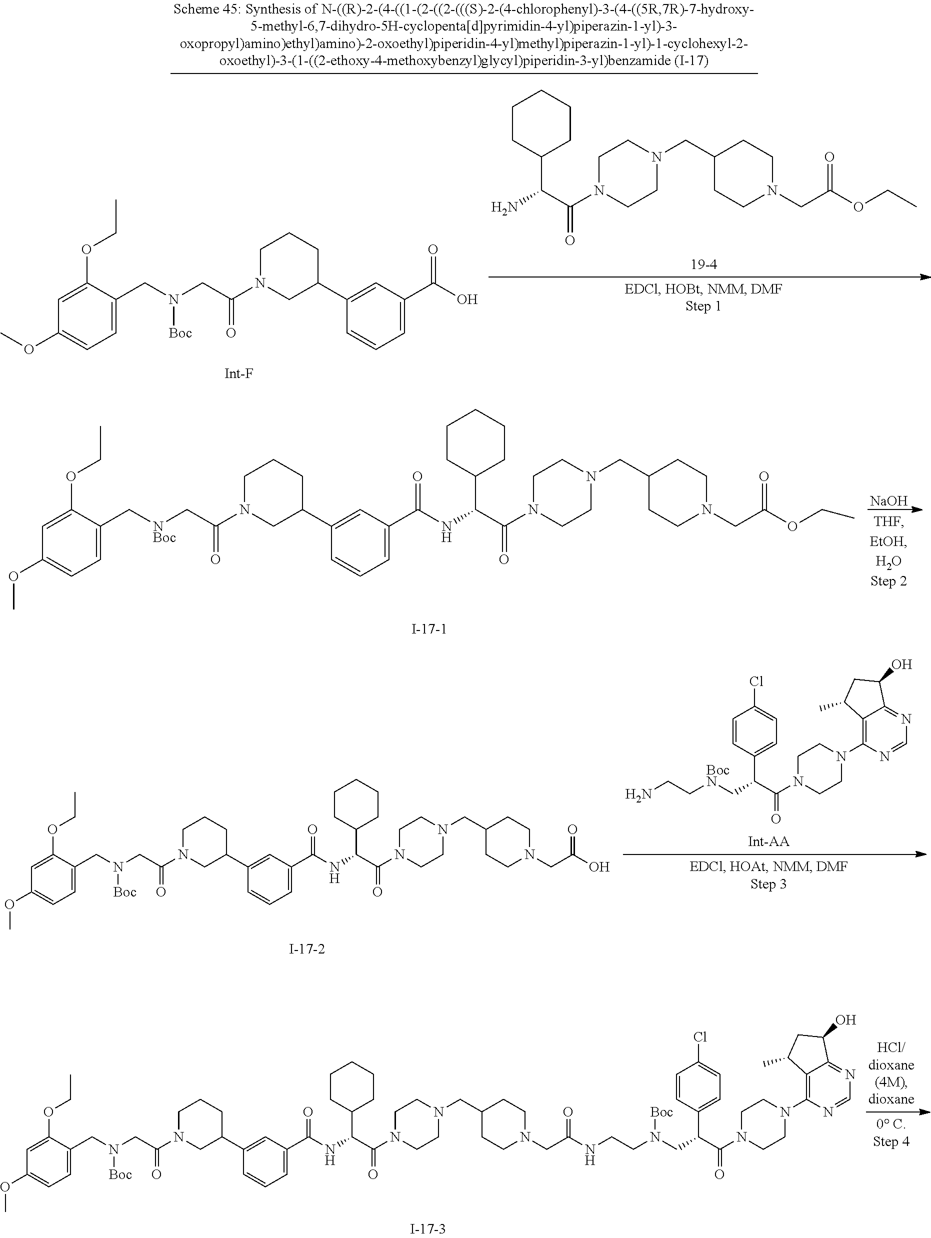

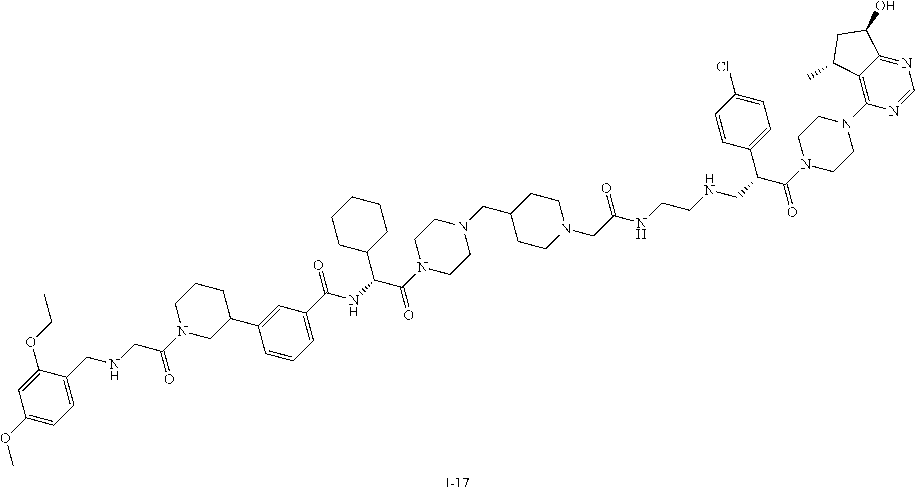

### Step 1: Synthesis of I-17-1

To a solution of 19-4 (140 mg, 342.65 μmol, 1 eq) and Int-F (198.49 mg, 376.92 μmol, 1.1 eq) in DMF (1.5 mL) was added EDCI (197.06 mg, 1.03 mmol, 3 eq), HOBt (46.30 mg, 342.65 μmol, 1 eq) and NMM (346.58 mg, 3.43 mmol, 376.72 μL, 10 eq). The mixture was stirred at 25° C. for 1 hr. The mixture was poured into water (10 mL) and extracted with EA (10 mL*3). The combined organic layers were concentrated to give a residue. The residue was purified by flash silica gel chromatography (12 g SepaFlash silica flash column; eluent: 0-37% MeOH/ethyl acetate; gradient at 20 mL/min) and the eluent was concentrated to give I-17-1 (210 mg, 208.36 μmol, 60.81% yield, 91% purity) as a yellow solid. LCMS (Method E): Rt=0.535 min, [M+H]⁺=917.8. SFC: Rt=1.294 min, ee=100%.

### Step 2: Synthesis of I-17-2

To a solution of I-17-2 (210 mg, 228.96 μmol, 1 eq) in THF (2 mL) was added a solution of NaOH (18.32 mg, 457.92 μmol, 2 eq) in H₂O (0.5 mL) and EtOH (2 mL). The mixture was stirred at 25° C. for 1 hr. The reaction mixture was concentrated under reduced pressure to give the crude product. The mixture was purified by reversed phased HPLC (FA) and then lyophilized to give I-17-2 (130 mg, 146.21 μmol, 63.86% yield, 99% purity) as a white solid. LCMS (Method E): Rt=0.535 min, M+H=889.6. SFC: Rt=1.341 min, ee>99%.

### Step 3: Synthesis of I-17-3

To a solution of I-17-2 (120 mg, 134.96 μmol, 1 eq) and Int-AA (75.46 mg, 134.96 μmol, 1 eq) in DMF (1.5 mL) was added EDCI (77.62 mg, 404.89 μmol, 3 eq), HOAt (18.37 mg, 134.96 μmol, 18.88 μL, 1 eq) and NMM (136.51 mg, 1.35 mmol, 148.38 μL, 10 eq). The mixture was stirred at 25° C. for 1 hr. The mixture was poured into water (10 mL) and extracted with EA (10 mL*3). The combined organic layers were concentrated to give a residue. The residue was purified by flash silica gel chromatography (12 g SepaFlash silica flash column; eluent: 0-37% MeOH/ethyl acetate; gradient at 20 mL/min) and the eluent was concentrated to give I-17-3 (55 mg, 32.30 μmol, 23.93% yield, 84% purity) as a yellow solid. LCMS (Method E): Rt=0.535 min, [M+H]⁺=1430.5.

### Step 4: Synthesis of I-17

To a solution of I-17-3 (55 mg, 38.46 μmol, 1 eq) in dioxane (0.5 mL) was added HCl/dioxane (4 M, 0.5 mL, 52.01 eq) at 0° C. The mixture was stirred at 0° C. for 0.5 hr. The reaction mixture was concentrated to give the crude product. The product was purified by prep-HPLC (column: Phenomenex luna C18 150 mm*25 mm*10 um; mobile phase: [water (FA):I]; gradient: 10%-40% IACN over 7 min) and the eluent was lyophilized to give I-17 (20.14 mg, 15.63 μmol, 40.64% yield, 99.02% purity, FA salt) as a colorless oil. LCMS (Method E): Rt=0.443 min, [M+H]⁺=1230.3. SFC: Rt=3.322 min and 5.939 min. 1H NMR (400 MHZ, methanol-d₄) δ 8.54 (s, 1-), 8.44-8.38 (m, 1-), 7.80-7.69 (m, 2-), 7.52-7.37 (m, 4-), 7.36-7.30 (m, 2-), 7.27-7.19 (m, 1-), 6.63-6.50 (m, 2H), 4.92 (s, 2-), 4.60-4.42 (m, 1-), 4.24-4.20 (m, 1-), 4.17-4.01 (m, 4-), 3.94-3.66 (m, 13-), 3.64-3.46 (m, 6-), 3.41-3.35 (m, 2-), 3.29-3.22 (m, 1-), 3.22-3.10 (m, 1H), 3.01 (s, 2-), 2.92-2.69 (m, 7-), 2.55-2.35 (m, 4-), 2.25-2.01 (m, 7-), 1.92-1.82 (m, 4H), 1.77 (d, J=12.0 Hz, 4H), 1.69 (d, J=12.0 Hz, 2-), 1.64-1.53 (m, 2-), 1.44-1.40 (m, 3-), 1.36-1.22 (m, 5-), 1.15-1.05 (m, 5H).

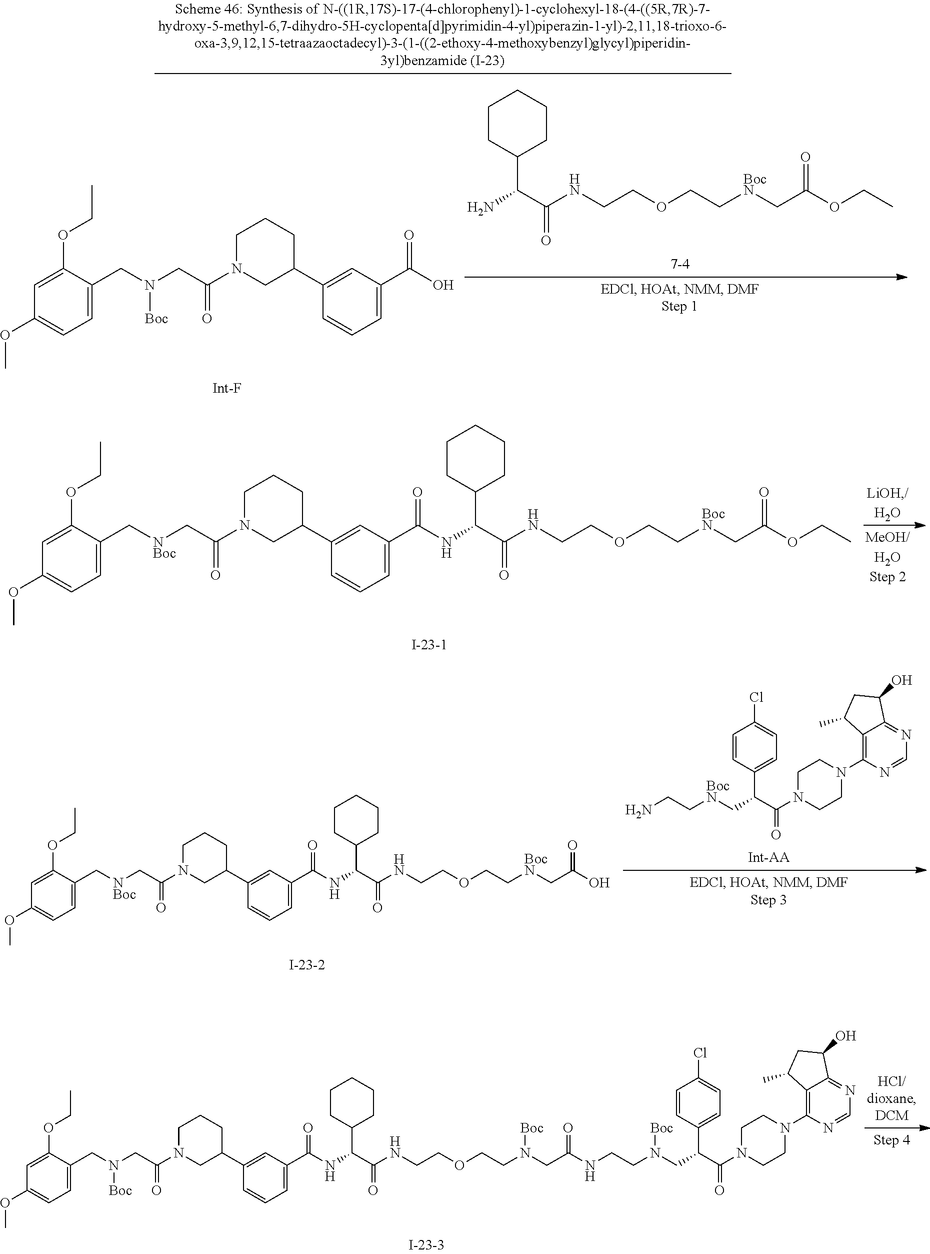

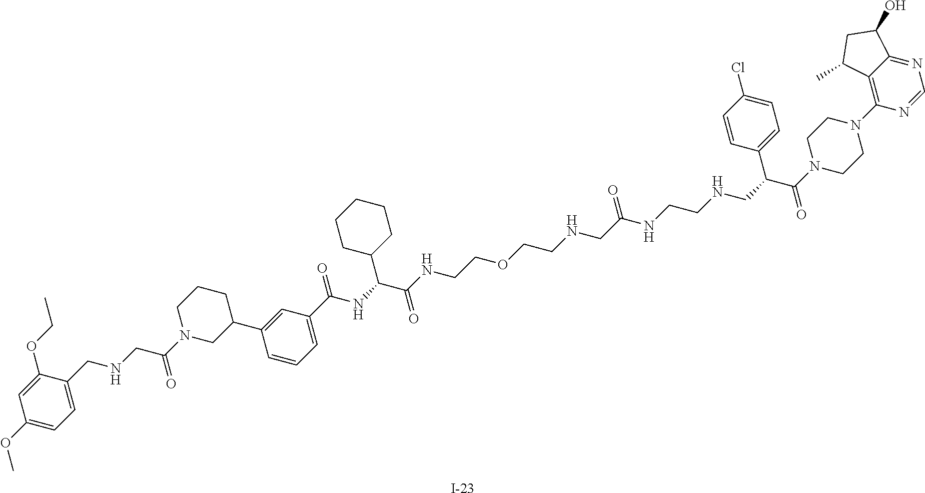

### Step 1: Synthesis of I-23-1

To a solution of Int-F (150 mg, 284.84 μmol, 1 eq), EDCI (109.21 mg, 569.67 μmol, 2 eq), HOAt (19.38 mg, 142.42 μmol, 19.92 μL, 0.5 eq), NMM (144.05 mg, 1.42 mmol, 156.58 μL, 5 eq) in DCM (2 mL) was added 7-4 (122.35 mg, 284.84 μmol, 1 eq). The mixture was stirred at 25° C. for 1 hr. The reaction mixture was poured into water (5 mL) and extracted with DCM (5 mL*2), the organic layer was washed with brine (5 mL), dried over Na₂SO₄, and concentrated to give the crude product. The crude product was purified by flash silica gel column chromatography (SiO₂, PE:EA=10:1 to 3:1) to give I-23-1 (200 mg, 203.80 μmol, 71.55% yield, 95.6% purity) as a colorless oil. LCMS (Method E): Rt=0.672 min, [M+H]⁺=938.8. SFC: Rt=2.74 min & 3.18 min.

### Step 2: Synthesis of I-23-2

To a solution of I-23-1 (80 mg, 85.27 μmol, 1 eq) in MeOH (0.5 mL) and H₂O (0.5 mL) was added LiOH·H₂O (10.74 mg, 255.82 μmol, 3 eq). The mixture was stirred at 25° C. for 16 hr. The reaction mixture was adjusted to pH=7 with 1N aq. HCl, and concentrated under vacuum to give I-23-2 (77 mg, 84.61 μmol, 93.4% yield, 94.2% purity) as a yellow solid. LCMS (Method E): Rt=0.631 min, [M+H]⁺=910.9.

### Step 3: Synthesis of I-23-3

To a solution of I-23-2 (77 mg, 84.61 μmol, 1 eq), EDCI (32.44 mg, 169.21 μmol, 2 eq), HOAt (5.76 mg, 42.30 μmol, 5.92 μL, 0.5 eq), NMM (42.79 mg, 423.03 μmol, 46.51 μL, 5 eq) in DCM (1 mL) was added Int-AA (56.76 mg, 101.53 μmol, 1.2 eq). The mixture was stirred at 25° C. for 1 hr. The reaction mixture was poured into water (5 mL) and extracted with DCM (5 mL*2), and the organic layer was washed with brine (5 mL), dried over Na₂SO₄, and concentrated to give the crude product. The crude product was purified by flash silica gel column chromatography (SiO₂, PE:EA=10:1 to 3:1) to give I-23-3 (65 mg, 42.55 μmol, 50.29% yield, 95.0% purity) as a colorless oil. LCMS (Method E): Rt=0.619 min, [M+H]⁺=1452.1.

### Step 4: Synthesis of I-23

To a solution of I-23-3 (60 mg, 41.35 μmol, 1 eq) in DCM (0.5 mL) was added HCl/dioxane (4 mol/L, 0.2 mL). The mixture was stirred at 0° C. for 0.5 hr. The reaction mixture was concentrated under vacuum to give the crude product. The crude product was purified by prep-HPLC (column: Phenomenex luna C18 150 mm*25 mm*10 um; mobile phase: [water (FA):ACN]; gradient: 12%-42% ACN over 7 min) and give I-23 (10.1 mg, 8.04 μmol, 19.45% yield, 95.3% purity; FA salt) as a white solid after lyophilization. LCMS (Method E): Rt=0.438 min, M+H=1150.6. SFC: Rt=2.54 min & 4.38 min. ¹H NMR (400 MHZ, methanol-d₄) δ 8.55 (b-s, 1H), 8.44-8.38-(m, 1H), 7.84-7.68-(m, 2H), 7.52-7.43-(m, 2H), 7.42-7.37-(m, 2H), 7.36-7.31-(m, 2H), 7.27-7.25-(m, 1H), 6.68-6.47-(m, 2H), 4.99-4.97-(m, 1H), 4.43-4.41-(m, 2H), 4.28-4.26-(m, 1H), 4.19-4.03-(m, 4H), 3.95-3.70-(m, 9H), 3.64-3.39 (m, 13H), 3.36 (b-s, 2H), 3.30-3.14-(m, 2H), 2.92-2.77-(m, 6H), 2.17-2.01-(m, 3H), 1.94-1.57-(m, 9H), 1.47-1.45-(m, 3H), 1.37-1.13 (m, 5H), 1.11 (br d, J=6.8 Hz, 3H).

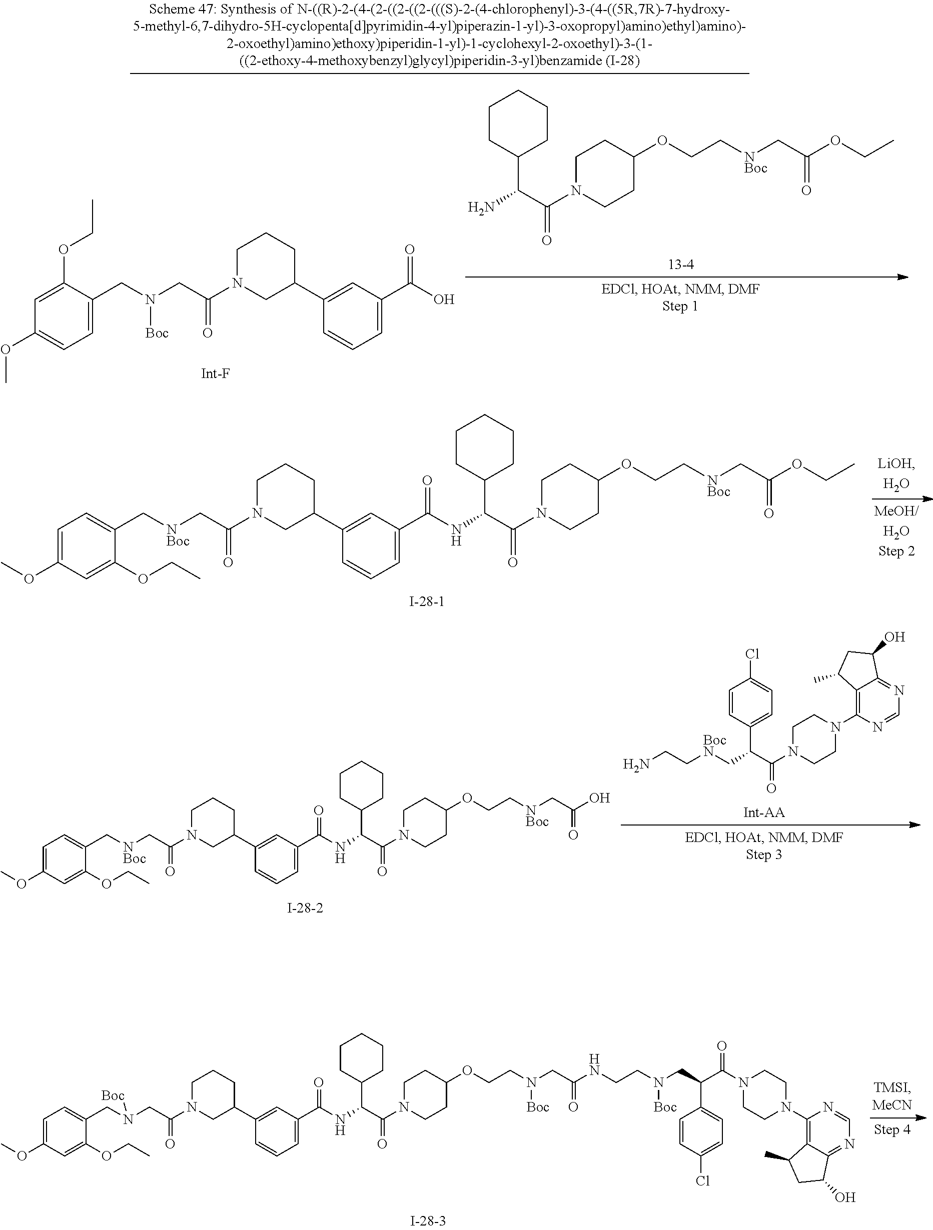

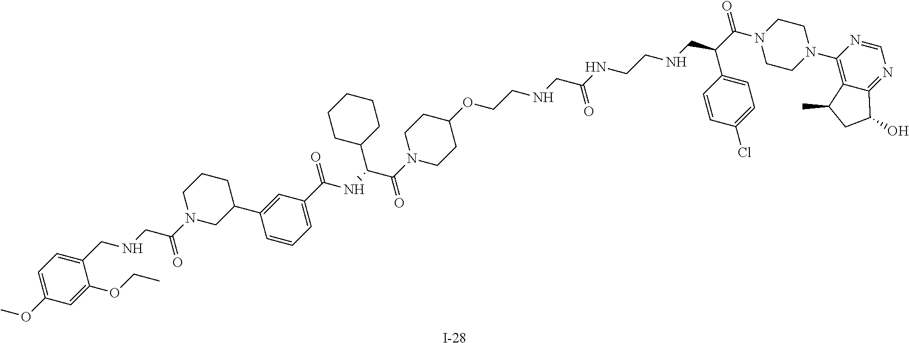

### Step 1: Synthesis of I-28-1

To a solution of Int-F (100 mg, 189.89 μmol, 1 eq), EDCI (72.80 mg, 379.78 μmol, 2 eq), HOAt (12.92 mg, 94.95 μmol, 13.28 μL, 0.5 eq), and NMM (96.03 mg, 949.45 μmol, 104.38 μL, 5 eq) in DCM (2 mL) was added 13-4 (98.09 mg, 208.88 μmol, 1.1 eq). The mixture was stirred at 25° C. for 1 hr. The reaction mixture was poured into water (5 mL) and extracted with DCM (5 mL*2), the organic layer was washed with brine (5 mL) dried over Na₂SO₄, and concentrated to give the crude product. The crude product was purified by flash silica gel column chromatography (SiO₂, PE:EA=10:1 to 3:1) and give I-28-1 (100 mg, 78.82 μmol, 41.51% yield, 77.1% purity) as a colorless oil. LCMS (Method E): Rt=0.713 min, [M+H]⁺=978.7.

### Step 2: Synthesis of I-28-2

To a solution of I-28-1 (80 mg, 81.78 μmol, 1 eq) in MeOH (0.5 mL) and H₂O (0.5 mL) was added LiOH·H₂O (10.30 mg, 245.82 μmol, 3 eq). The mixture was stirred at 25° C. for 16 hr. The reaction mixture was adjusted to pH=7 with 1N HCl, and concentrated under vacuum to give I-28-2 (77 mg, 76.18 μmol, 93.15% yield, 94.0% purity) as a white solid. LCMS (Method E): Rt=0.653 min, [M-tBu]⁺=892.7. SFC: Rt=2.57 min & 2.77 min.

### Step 3: Synthesis of I-28-3

To a solution of I-28-2 (77 mg, 81.04 μmol, 1 eq), EDCI (31.07 mg, 162.08 μmol, 2 eq), HOAt (5.52 mg, 40.52 μmol, 5.92 μL, 0.5 eq), and NMM (40.98 mg, 405.19 μmol, 44.55 μL, 5 eq) in DCM (1 mL) was Int-AA (54.37 mg, 97.25 μmol, 1.2 eq). The mixture was stirred at 25° C. for 1 hr. The reaction mixture was poured into water (5 mL), extracted with DCM (5 mL*2), and the organic layer was washed with brine (5 mL), dried over Na₂SO₄ and concentrated to give the crude product. The crude product was purified by flash silica gel column chromatography (SiO₂, PE:EA=10:1 to 3:1) to yield I-28-3 (70 mg, 35.96 μmol, 44.37% yield, 76.6% purity) as a colorless oil. LCMS (Method E): Rt=0.620 min, [M+H]⁺=1491.0.

### Step 4: Synthesis of I-28

To a solution of I-28-3 (65 mg, 43.59 μmol, 1 eq) in MeCN (0.5 mL) was added TMSI (87.22 mg, 435.88 μmol, 59.33 μL, 10 eq). Then the mixture was stirred at 0° C. for 0.5 hr. The reaction mixture was concentrated under vacuum to give the crude product. The crude product was purified by prep-HPLC (column: C18 150 mm*30 mm; mobile phase: [water (FA):ACN]; gradient: 15%-45% ACN over 7 min) to yield I-28 (10.1 mg, 8.04 μmol, 19.45% yield, 95.3% purity, FA salt) as a white solid after lyophilization.

LCMS (Method E): Rt=0.428 min, [M+H]⁺=1190.7. SFC: Rt=3.32 min & 5.69 min. ¹H NMR (400 MHZ, methanol-d₄) δ 8.55 (br s, 1H), 8.43 (d, J=2.8 Hz, 1H), 7.75 (br d, J=13.2 Hz, 2H), 7.53-7.31 (m, 6H), 7.29-7.19 (m, 1H), 6.71-6.41 (m, 2H), 5.02-4.94 (m, 2H), 4.62-4.44 (m, 1H), 4.25 (br d, J=4.8 Hz, 1H), 4.18-4.00 (m, 5H), 3.97-3.70 (m, 10H), 3.69-3.44 (m, 10H), 3.43-3.34 (m, 4H), 3.28-3.07 (m, 2H), 2.94-2.68 (m, 7H), 2.18-1.99 (m, 4H), 1.97-1.83 (m, 5H), 1.82-1.56 (m, 7H), 1.55-1.21 (m, 7H), 1.19-1.07 (m, 5H).

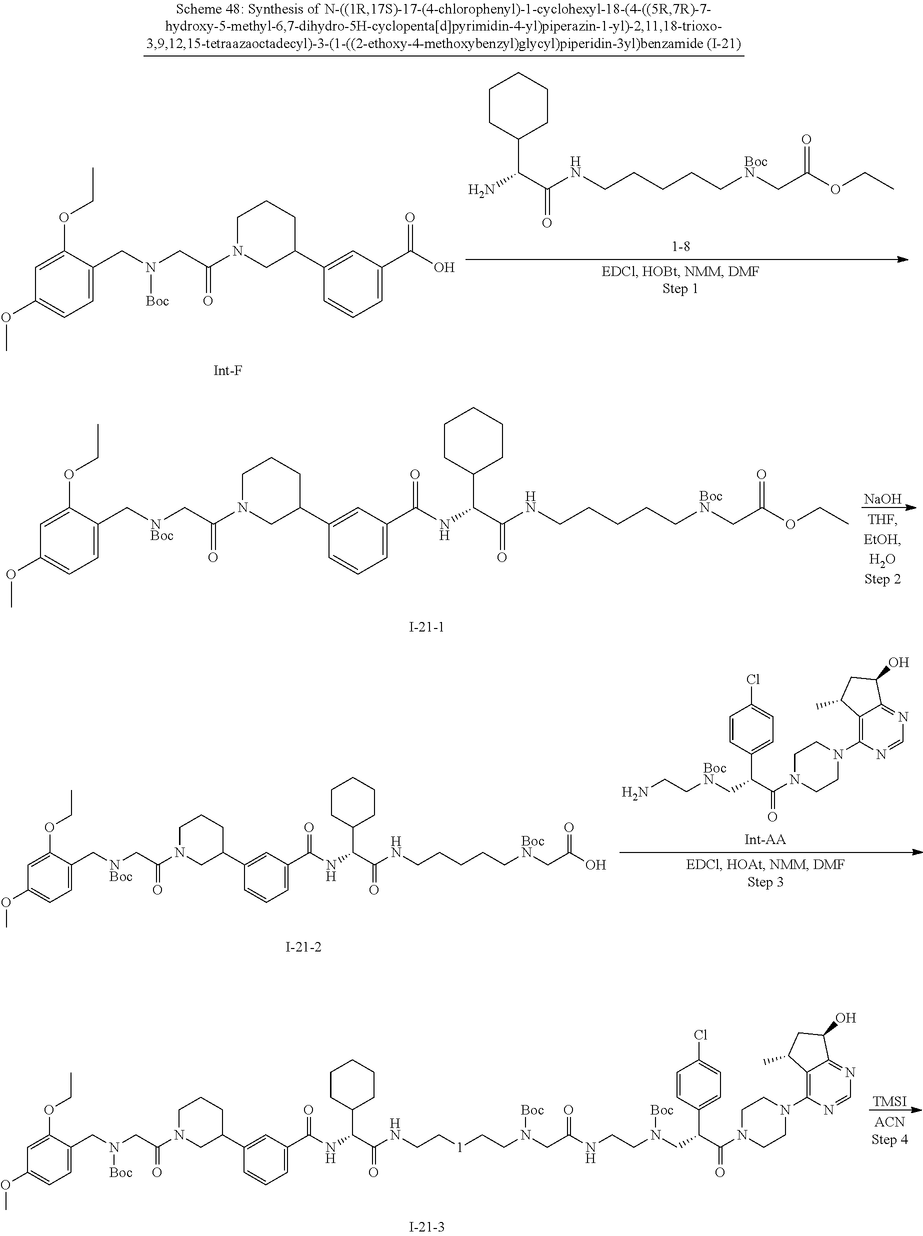

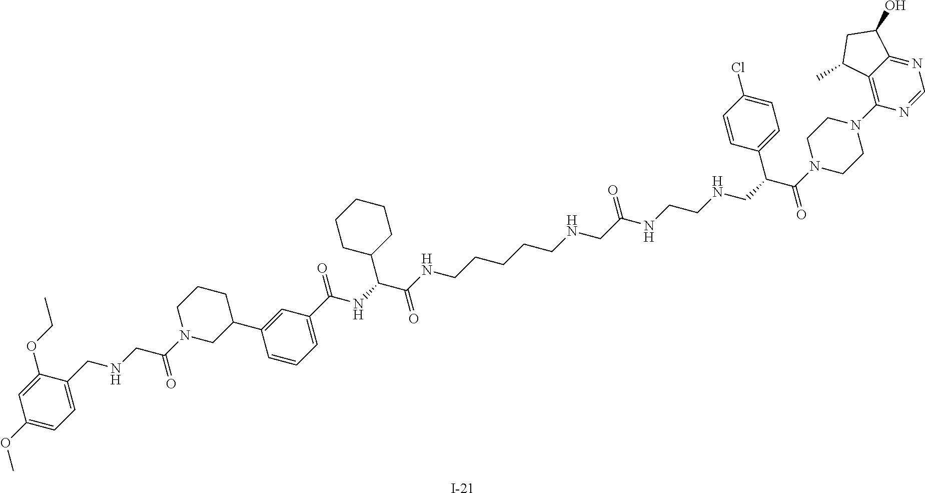

### Step 1: Synthesis of I-21-1

To a solution of ethyl 1-8 (133.97 mg, 313.32 μmol, 1.1 eq) and Int-F (150 mg, 284.84 μmol, 1 eq) in DMF (2 mL) was added EDCI (163.81 mg, 854.51 μmol, 3 eq), HOBt (38.49 mg, 284.84 μmol, 1 eq) and NMM (288.10 mg, 2.85 mmol, 313.15 μL, 10 eq). The mixture was stirred at 25° C. for 1 hr. The mixture was poured into water (10 mL) and extracted with EA (10 mL*3). The combined organic layers were concentrated to give a residue. The residue was purified by flash silica gel chromatography (12 g SepaFlash silica flash column; eluent: 0-100% ethyl acetate/petroleum ether; gradient at 20 mL/min) and the eluent was concentrated to give I-28-1 (250 mg, 256.36 μmol, 90.0% yield, 96% purity) as a yellow solid. LCMS (Method E): Rt=0.677 min, [M+H]⁺=936.5.

### Step 2: Synthesis of I-28-2

To a solution of I-280-1 (180 mg, 192.27 μmol, 1 eq) in THF (2 mL) was added a solution of NaOH (15.38 mg, 384.54 μmol, 2 eq) in H₂O (0.5 mL) and EtOH (2 mL). The mixture was stirred at 25° C. for 1 hr. The reaction mixture was concentrated under reduced pressure to give the crude product. The crude product was purified by reversed phased HPLC (FA), concentrated, and then extracted with EA (10 mL*2), dried over anhydrous sodium sulfate, filtered and concentrated to give I-28-2 (160 mg, 169.14 μmol, 87.97% yield, 96% purity) as a white solid. LCMS (Method E): Rt=0.643 min, M+H=908.7. SFC: Rt=0.644 min, ee>99%.

### Step 3: Synthesis of I-28-3

To a solution of I-28-2 (80 mg, 88.09 μmol, 1 eq) and Int-AA (49.25 mg, 88.09 μmol, 1 eq) in DMF (1 mL) was added EDCI (50.66 mg, 264.28 μmol, 3 eq), HOAt (11.99 mg, 88.09 μmol, 12.32 μL, 1 eq) and NMM (89.10 mg, 880.93 μmol, 96.85 μL, 10 eq). The mixture was stirred at 25° C. for 1 hr. The mixture was poured into water (10 mL) and extracted with EA (10 mL*3). The combined organic layers were concentrated to give a residue. The residue was purified by flash silica gel chromatography (12 g SepaFlash silica flash column; eluent: 0-37% MeOH/ethyl acetate; gradient at 20 mL/min) to give I-28-3 (120 mg, 76.48 μmol, 86.81% yield, 92.36% purity) as a yellow solid. LCMS (Method E): Rt=0.618 min, [M+H]⁺=1449.9.

### Step 4: Synthesis of I-21

To a solution of I-28-3 (50 mg, 34.50 μmol, 1 eq) in ACN (1 mL) was added TMSI (69.04 mg, 345.02 μmol, 46.96 μL, 10 eq) at 0° C. The mixture was stirred at 0° C. for 0.5 hr. The reaction mixture was concentrated to give the crude product. The product was purified by prep-HPLC (column: Phenomenex luna C18 150 mm*25 mm*10 um; mobile phase: [water (FA):ACN]; gradient: 15%-45% ACN over 7 min) and the eluent was lyophilized to give I-21 (15.78 mg, 13.21 μmol, 38.28% yield, 99% purity, FA salt) as a colorless oil. LCMS (Method E): Rt=0.418 min, [M+H]⁺=1148.8. SFC: Rt=2.478 min and 4.582 min. 1H NMR (400 MHZ, methanol-d₄) δ 8.51 (s, 2H), 8.44-8.38 (m, 1H), 7.79-7.68 (m, 2H), 7.51-7.38 (m, 4H), 7.36-7.24 (m, 3H), 6.64-6.52 (m, 2H), 5.01-4.91 (m, 1H), 4.60-4.45 (m, 1H), 4.37-4.28 (m, 2H), 4.21-4.08 (m, 4H), 4.03-3.85 (m, 3H), 3.80 (d, J=4.0 Hz, 3H), 3.76-3.70 (m, 1H), 3.71 (s, 2H), 3.64-3.51 (m, 5H), 3.51-3.37 (m, 5H), 3.27-3.11 (m, 4H), 2.98-2.90 (m, 3H), 2.89-2.67 (m, 4H), 2.17-2.09 (m, 1H), 2.04-2.00 (m, 2H), 1.93-1.82 (m, 4H), 1.78 (d, J=12.8 Hz, 2H), 1.73-1.61 (m, 5H), 1.57 (d, J=6.0 Hz, 2H), 1.45-1.40 (m, 5H), 1.33-1.19 (m, 3H), 1.18-1.04 (m, 5H).

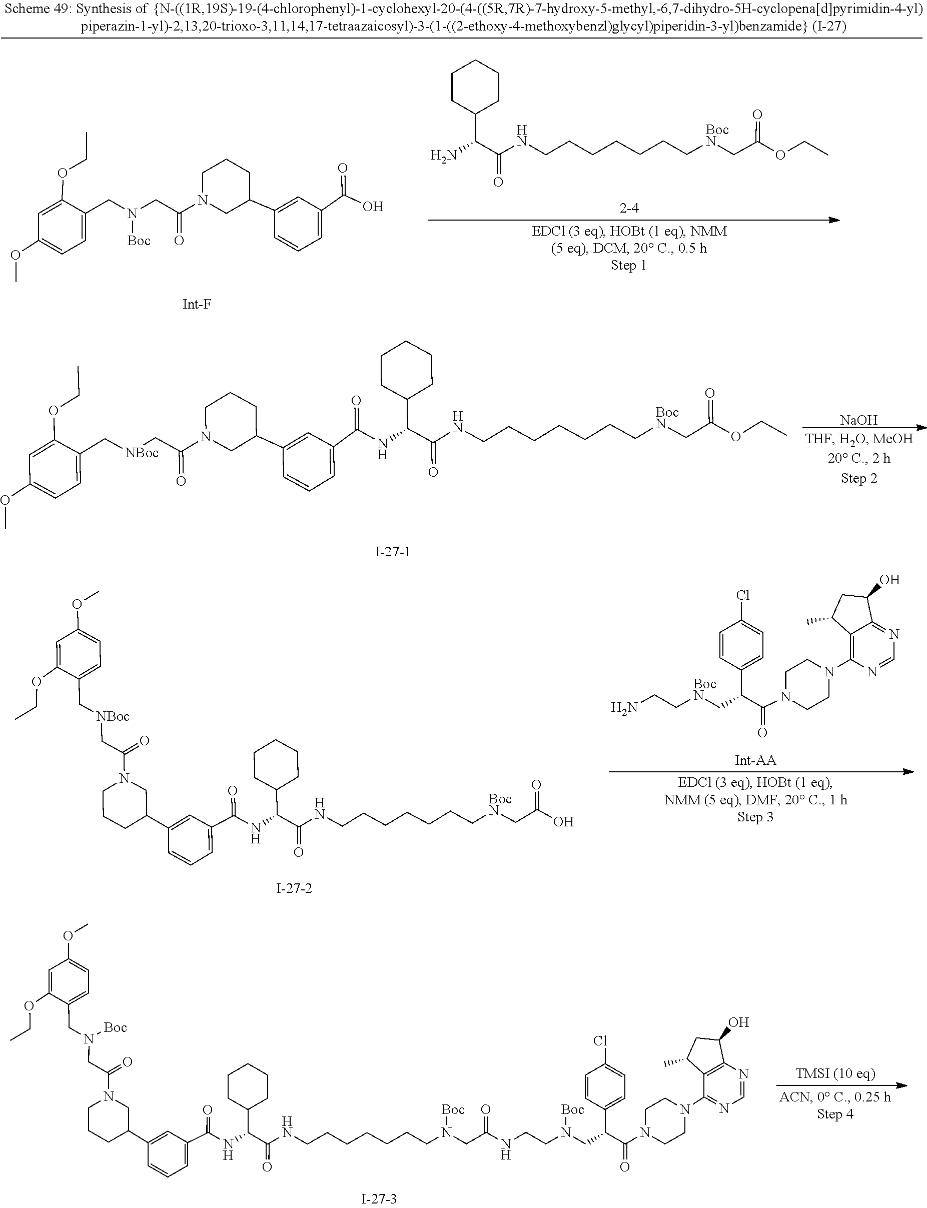

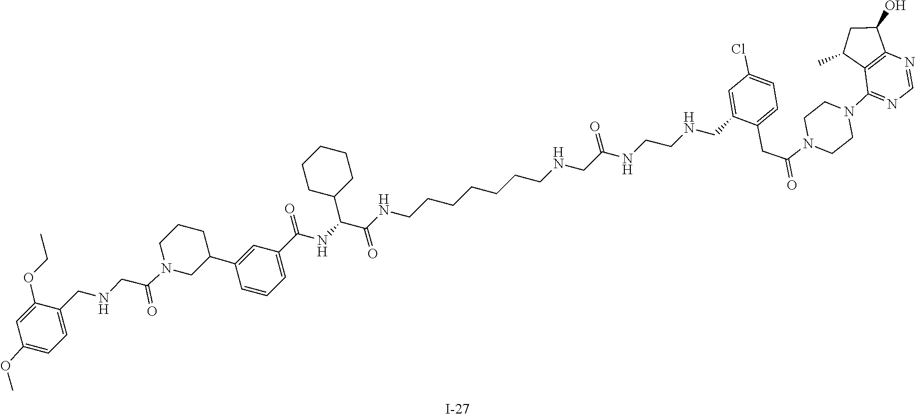

### Step 1: Synthesis of I-27-1

To a solution of 2-4 (100 mg, 219.48 μmol, 1 eq), Int-F (98.24 mg, 186.56 μmol, 0.85 eq), EDCI (126.22 mg, 658.43 μmol, 3 eq) and HOBt (29.66 mg, 219.48 μmol, 1 eq) in DCM (1 mL) was added NMM (111.00 mg, 1.10 mmol, 120.65 μL, 5 eq) and the mixture was stirred at 20° C. for 0.5 h. The mixture was purified by flash silica gel chromatography (8 g SepaFlash silica flash column; eluent: 0-60% ethyl acetate/petroleum ether; gradient at 36 mL/min) and the eluent was concentrated. I-27-1 (80 mg, 69.69 μmol, 31.75% yield, 84% purity) was obtained as a colorless oil. LCMS (Method E): Rt=0.705 min, [M+H]⁺=964.7.

### Step 2: Synthesis of I-27-2

To a solution of I-27-1 (80 mg, 69.69 μmol, 1 eq) in THF (1 mL) and MeOH (0.5 mL) was added NaOH (13.94 mg, 348.46 μmol, 5 eq) in H₂O (0.5 mL) and then the mixture was stirred at 20° C. for 2 h. The mixture was poured into water (5 mL) and extracted with EA (5 mL*5). The combined organic layers were dried over anhydrous Na₂SO₄, filtered, and concentrated. I-27-2 (80 mg, crude) was obtained as a white solid. LCMS (Method E): Rt=0.658 min, [M+H]⁺=936.6.

### Step 3: Synthesis of I-27-3

To a mixture of 11-2 (40 mg, 33.33 μmol, 1 eq), EDCI (19.17 mg, 99.98 μmol, 3 eq) and HOBt (4.50 mg, 33.33 μmol, 1 eq) in DMF (0.5 mL) was added NMM (16.85 mg, 166.63 μmol, 18.32 μL, 5 eq) and the mixture was stirred at 20° C. for 5 min. Then Int-AA (18.63 mg, 33.33 μmol, 1 eq) was added. The mixture was stirred at 20° C. for 1 hr. The residue was purified by prep-HPLC (column: YMC-Actus Triart C18 150 mm*30 mm*7 um; mobile phase: [water (FA):ACN]; gradient: 58%-88% ACN over 10 min) and the eluent was concentrated to remove ACN. The pH was adjusted to 8-9 with a solution of saturated NaHCO₃ and then extracted with EA (5 mL*5). The combined organic layers were dried over anhydrous Na₂SO₄, filtered and concentrated. I-27-3 (35 mg, 23.69 μmol, 71.09% yield, 99% purity) was obtained as a yellow oil. LCMS (Method E): Rt=0.645 min, [M+H]⁺=1478.1.

SFC: Rt=2.113 & 2.624 min.

### Step 4: Synthesis of I-27

To a solution of I-27-3 (35 mg, 23.69 μmol, 1 eq) in ACN (2.5 mL) was added TMSI (47.41 mg, 236.92 μmol, 32.25 μL, 10 eq) at 0° C., and then the mixture was stirred at 0° C. for 0.25 h. The mixture was poured into water (4 mL) and adjusted to pH=8 with a solution of saturated NaHCO₃ and extracted with CHCl₃/isopropanol=3/1 (4 mL*3). The combined organic layers were dried over anhydrous Na₂SO₄, filtered and concentrated to give a residue. The residue was purified by prep-HPLC (column: YMC-Actus Triart C18 150 mm*30 mm*7 um; mobile phase: [water (FA):ACN]; gradient: 13%-43% ACN over 10 min), followed by lyophilization. I-27 (11.31 mg, 9.53 μmol, 40.20% yield, 99.12% purity) was obtained as a white solid. LCMS (Method E): Rt=0.432 min, [M+H]⁺=1177.2. SFC: Rt=2.545 min, 4.743 min. 1H NMR (400 MHZ, methanol-d₄) δ=8.41 (s, 1H), 7.82-7.66 (m, 2H), 7.56-7.37 (m, 4H), 7.37-7.32 (m, 2H), 7.31-7.23 (m, 1H), 6.62 (d, J=7.2 Hz, 1H), 6.59-6.52 (m, 1H), 4.99-4.95 (m, 1H), 4.62-4.43 (m, 1H), 4.39-4.26 (m, 2H), 4.22-3.86 (m, 7H), 3.81 (d, J=4.0 Hz, 3H), 3.78-3.66 (m, 3H), 3.65-3.35 (m, 11H), 3.29-3.12 (m, 4H), 3.04-2.66 (m, 7H), 2.19-2.00 (m, 3H), 1.95-1.75 (m, 6H), 1.74-1.59 (m, 5H), 1.58-1.51 (m, 2H), 1.50-1.43 (m, 3H), 1.42-1.34 (m, 6H), 1.33-1.13 (m, 4H), 1.11 (d, J=6.8 Hz, 3H).

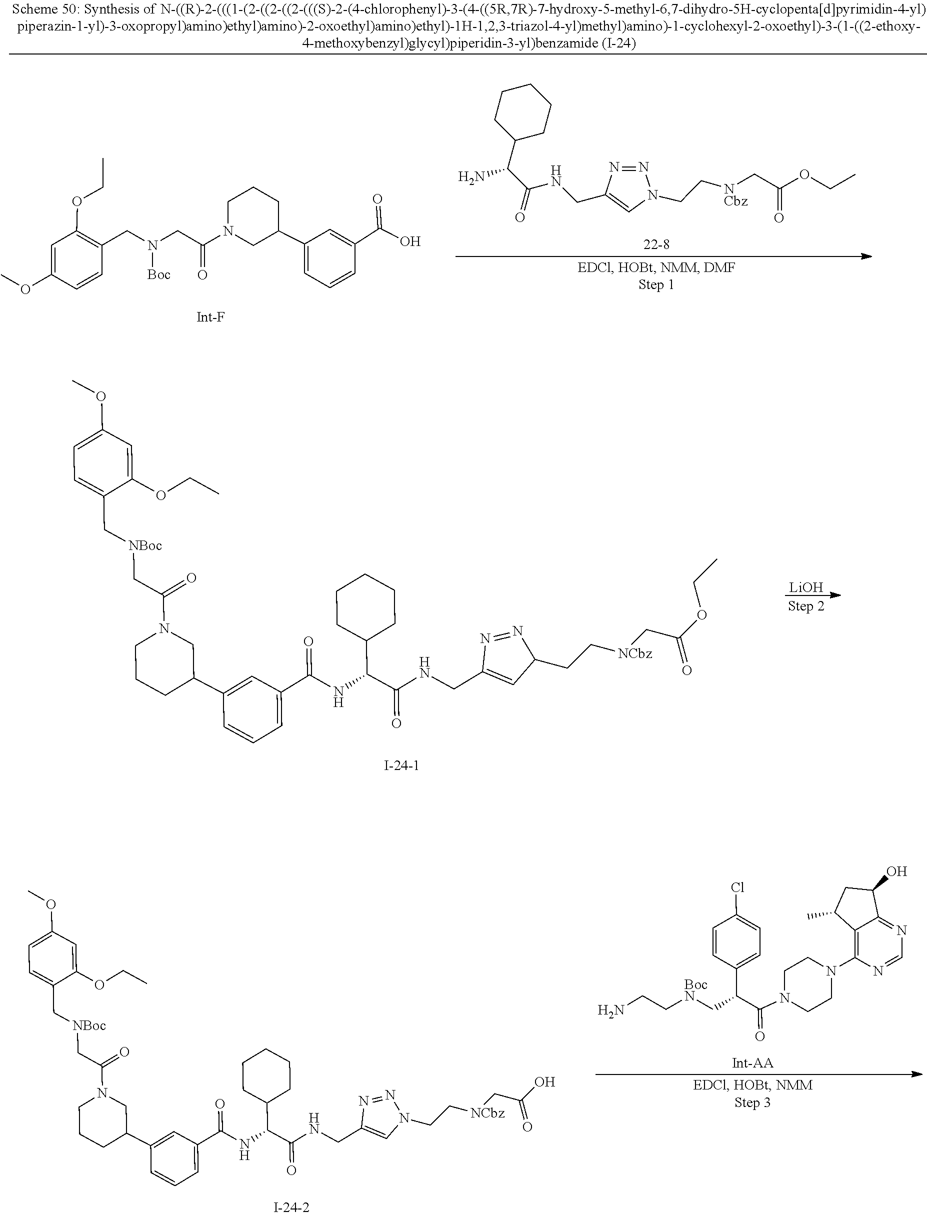

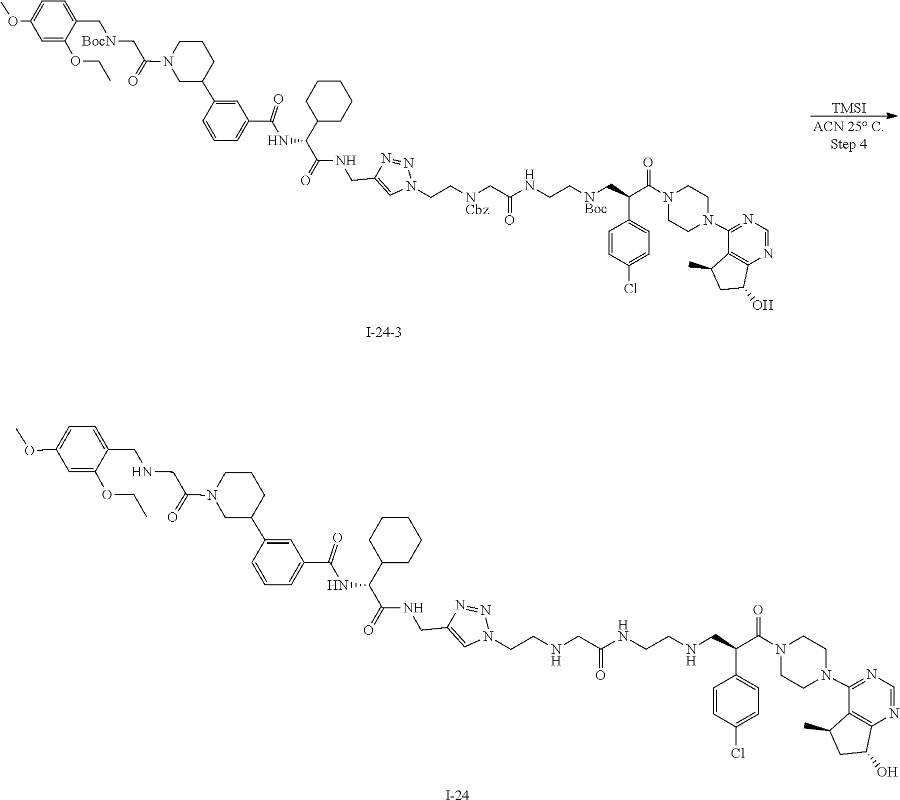

### Step 1: Synthesis of I-24-1

To a solution of 22-8 (40 mg, 74.48 μmol, 1 eq, HCl) and Int-F (43.15 mg, 81.93 μmol, 1.1 eq) in DMF (0.5 mL) was added EDCI (42.83 mg, 223.44 μmol, 3 eq), HOBt (15.10 mg, 111.72 μmol, 1.5 eq) and NMM (75.34 mg, 744.81 μmol, 81.89 μL, 10 eq). The mixture was stirred at 25° C. for 12 hr. The reaction was poured into water (2 ml), extracted with EA (2 ml*3), washed with brine, and the organic layer was separated, dried with anhydrous Na₂SO₄, filtered and concentrated under vacuum to give I-24-2 (65 mg, 58.61 μmol, 78.69% yield, 91% purity) as a yellow solid. LCMS (Method E): Rt=0.652 min, [M+H-Boc]⁺=909.5. LCMS (Method E): Rt=0.638 min, [M+H]⁺=1009.7. SFC: Rt=1.775 min, 2.669 min.

### Step 2: Synthesis of I-24-2

To a solution of 12-1 (60 mg, 59.45 μmol, 1 eq) in MeOH (0.6 mL) and H₂O (0.3 mL) was added LiOH·H₂O (4.99 mg, 118.91 μmol, 2 eq). The mixture was stirred at 25° C. for 1 hr. The reaction was acidified with aq. HCl (1 M) to adjust the pH to 7 and then concentrated under vacuum to give I-24-2 (40 mg, 37.92 μmol, 63.77% yield, 93% purity). I-24-2 was used in the next step without further purification. LCMS (Method E): Rt=0.595 min, [M+H]⁺=981.6. SFC: Rt=1.185 min, 1.368 min.

### Step 3: Synthesis of I-24-3

To a solution of I-24-2 (40 mg, 40.77 μmol, 1 eq) in DMF (1 mL) was added EDCI (23.45 mg, 122.31 μmol, 3 eq), HOBt (8.26 mg, 61.15 μmol, 1.5 eq) NMM (41.24 mg, 407.69 μmol, 44.82 μL, 10 eq), and Int-AA (25.07 mg, 44.85 μmol, 1.1 eq) and the mixture was stirred at 25° C. for 1 hr. The reaction was poured into water (5 ml), extracted with EA (2 ml*3), washed with brine and concentrated under vacuum. The residue was purified by flash silica gel chromatography to give I-24-3 (30 mg, 17.15 μmol, 42.06% yield, 87% purity) as a white solid. LCMS (Method E): Rt=0.618 min, [M+H]⁺=1522.1.

### Step 4: Synthesis of I-24

To a solution of I-24-3 (25 mg, 16.42 μmol, 1 eq) in ACN (0.5 mL) was added TMSI (39.43 mg, 197.08 μmol, 26.83 μL, 12 eq). The mixture was stirred at 25° C. for 12 hr. The reaction was poured into water (2 ml), extracted with EA (3 ml*3) and the organic layer was separated and concentrated to give the crude product. The crude product was purified by prep-HPLC (FA condition column: C18 150 mm*30 mm; mobile phase: [water (FA):ACN]; gradient: 12%-42% ACN over 7 min) and lyophilized to give I-24 (3.5 mg, 2.95 μmol, 17.94% yield, 100% purity) as a white solid. LCMS (Method E): Rt=0.426 min, [M+H]⁺=1187.9. SFC: Rt=3.726 min, 8.630 min. ¹H NMR (400 MHZ, methanol-d₄) δ 8.40 (s, 1H), 7.93 (d, J=3.2 Hz, 1H), 7.80-7.68 (m, 2H), 7.50-7.20 (m, 7H), 6.64-6.45 (m, 2H), 4.97-4.96 (m, 2H), 4.58-4.43 (m, 5H), 4.40-4.33 (m, 1H), 4.28-4.26 (m, 1H), 4.17-4.04 (m, 4H), 4.01-3.82 (m, 3H), 3.82-3.75 (m, 4H), 3.75-3.63 (m, 3H), 3.62-3.36 (m, 6H), 3.21-3.12 (m, 3H), 3.05-3.03 (m, 2H), 2.92-2.69 (m, 5H), 2.16-1.83 (m, 7H), 1.82-1.53 (m, 6H), 1.46-1.42 (m, 3H), 1.34-1.12 (m, 5H), 1.09 (d, J=7.2 Hz, 3H).

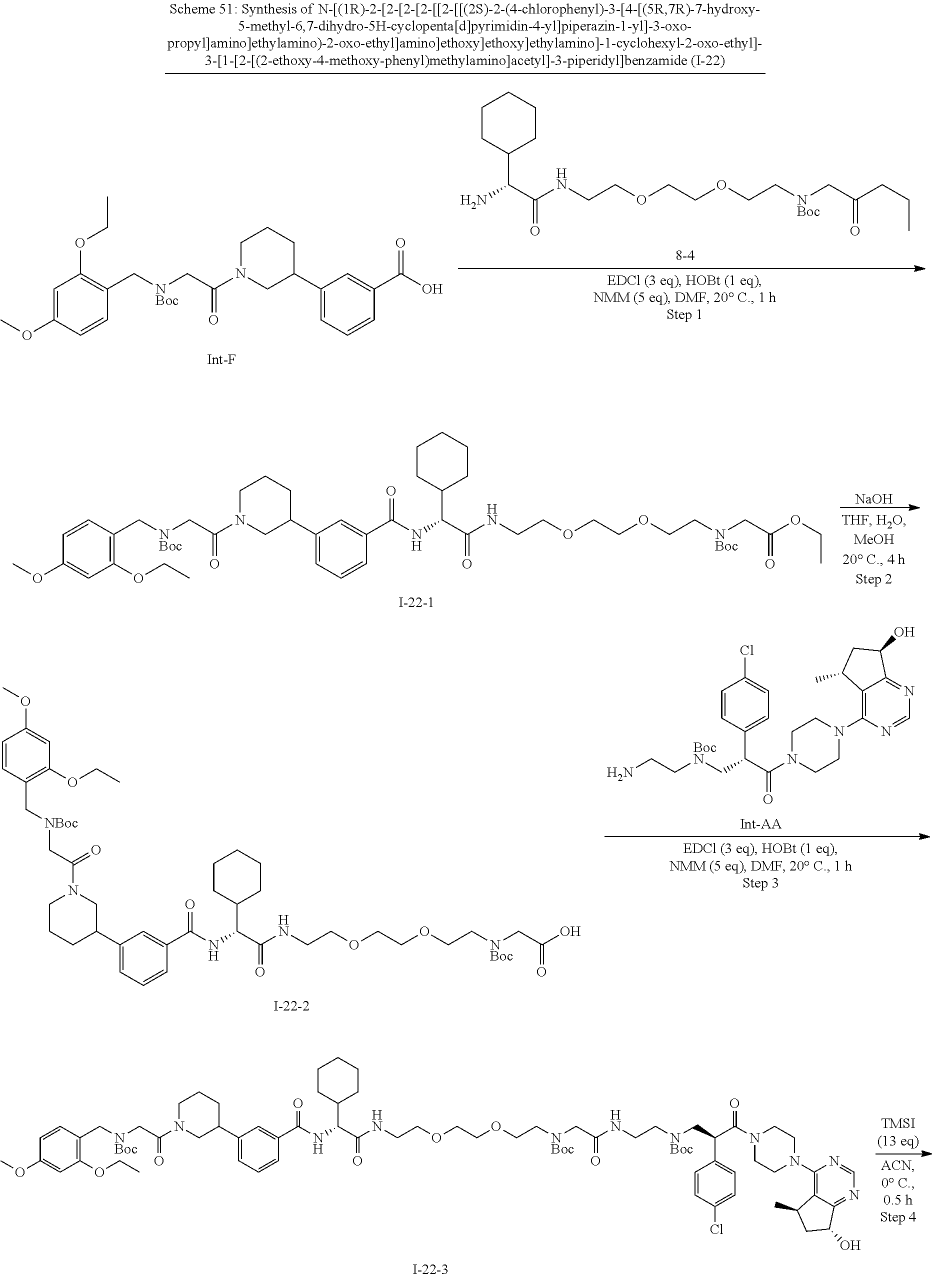

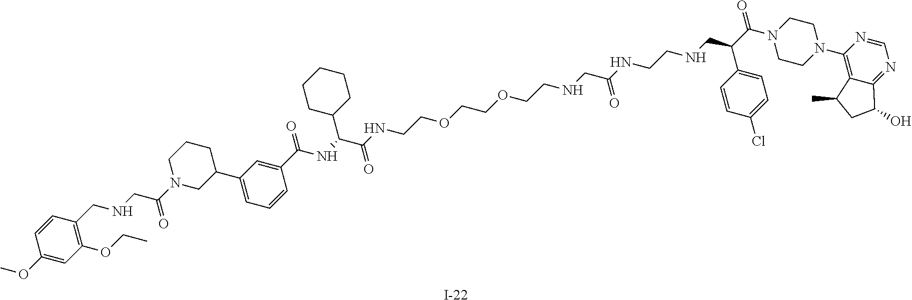

### Step 1: Synthesis of I-22-1

To a mixture of Int-F (30.02 mg, 57.01 μmol, 0.9 eq), EDCI (36.43 mg, 190.03 μmol, 3 eq) and HOBt (8.56 mg, 63.34 μmol, 1 eq) in DCM (0.5 mL) was added NMM (32.04 mg, 316.72 μmol, 34.82 μL, 5 eq) and 8-4 (30 mg, 63.34 μmol, 1 eq). Then the mixture was stirred at 20° C. for 1 hr. The mixture was purified by flash silica gel chromatography (4 g SepaFlash silica flash column; eluent: 0-100% ethyl acetate/petroleum ether; gradient at 18 mL/min) to afford I-22-3 (40 mg, 40.72 μmol, 64.29% yield) was obtained as a colorless oil. LCMS (Method E): Rt=0.668 min, [M+H]⁺=982.6. SFC: Rt=3.088 min, 3.544 min.

### Step 2: Synthesis of I-22-2

To a solution of I-22-1 (40 mg, 40.72 μmol, 1 eq) in THF (0.5 mL) and MeOH (0.3 mL) was added NaOH (8.14 mg, 203.62 μmol, 5 eq) in H₂O (0.3 mL) and then the mixture was stirred at 20° C. for 4 hrs. The mixture was poured into water (3 mL) and extracted with EA (3 mL*4). The combined organic layers were dried over anhydrous Na₂SO₄, filtered and concentrated to give I-22-2 (40 mg, crude) was obtained as colorless oil. LCMS (Method E): Rt=0.627 min, [M+H]⁺=954.6. SFC: Rt=1.275 min, 1.634 min.

### Step 3: Synthesis of I-22-3

To a mixture of I-22-2 (40 mg, 41.92 μmol, 1 eq), EDCI (24.11 mg, 125.77 μmol, 3 eq) and HOBt (5.66 mg, 41.92 μmol, 1 eq) in DMF (0.5 mL) was added NMM (21.20 mg, 209.61 μmol, 23.05 μL, 5 eq) and the mixture was stirred at 20° C. for 5 min. Then Int-AA (21.09 mg, 37.73 μmol, 0.9 eq) was added. The mixture was stirred at 20° C. for 1 hr. The mixture was poured into water (3 mL) and extracted with EA (3 mL*3). The combined organic layers were dried over anhydrous Na₂SO₄, filtered and then concentrated to give a residue. The residue was purified by flash silica gel chromatography (4 g SepaFlash silica flash column; eluent: 0-20% MeOH/DCM; gradient at 18 mL/min) to afford I-22-3 (50 mg, 25.41 μmol, 60.62% yield, 76% purity) was obtained as a yellow oil. LCMS (Method E): Rt=0.631 min, M+H=1495.9.

### Step 4: Synthesis of I-22

To a solution of I-22-3 (40 mg, 20.33 μmol, 1 eq) in ACN (1.2 mL) was added TMSI (52.89 mg, 264.31 μmol, 35.98 μL, 13 eq) at 0° C., and then the mixture was stirred at 0° C. for 0.5 h. The mixture was quenched by addition of a solution of saturated NaHCO₃ (2 mL) and extracted with CHCl₃/isopropanol=3:1 (2 mL*3). The combined organic layers were dried over anhydrous Na₂SO₄, filtered and concentrated to give the crude product. The crude product was purified by prep-HPLC (column: Phenomenex luna C18 150 mm*25 mm*10 um; mobile phase: [water (FA):ACN]; gradient: 5%-35% ACN over 10 min) and then lyophilized to afford I-22 (8.83 mg, 7.12 μmol, 35.00% yield, 99% purity, FA salt) as a white solid. LCMS (Method E): Rt=0.424 min, M+H=1194.8. SFC: Rt=2.805 min, 5.170 min. ¹H NMR (400 MHz, methanol-d₄) δ=8.51 (s, 1H), 8.41 (s, 1H), 7.81-7.66 (m, 2H), 7.56-7.20 (m, 7H), 6.67-6.48 (m, 2H), 4.99-4.94 (m, 1H), 4.60-4.43 (m, 1H), 4.42-4.36 (m, 1H), 4.35-4.27 (m, 1H), 4.21-4.08 (m, 4H), 4.06-3.84 (m, 3H), 3.80 (d, J=4.4 Hz, 3H), 3.76-3.66 (m, 3H), 3.65-3.35 (m, 20H), 3.28-3.11 (m, 3H), 2.99-2.70 (m, 7H), 2.17-2.08 (m, 1H), 2.08-2.01 (m, 2H), 1.94-1.58 (m, 9H), 1.50-1.41 (m, 3H), 1.33-1.14 (m, 4H), 1.10 (d, J=6.8 Hz, 3H).

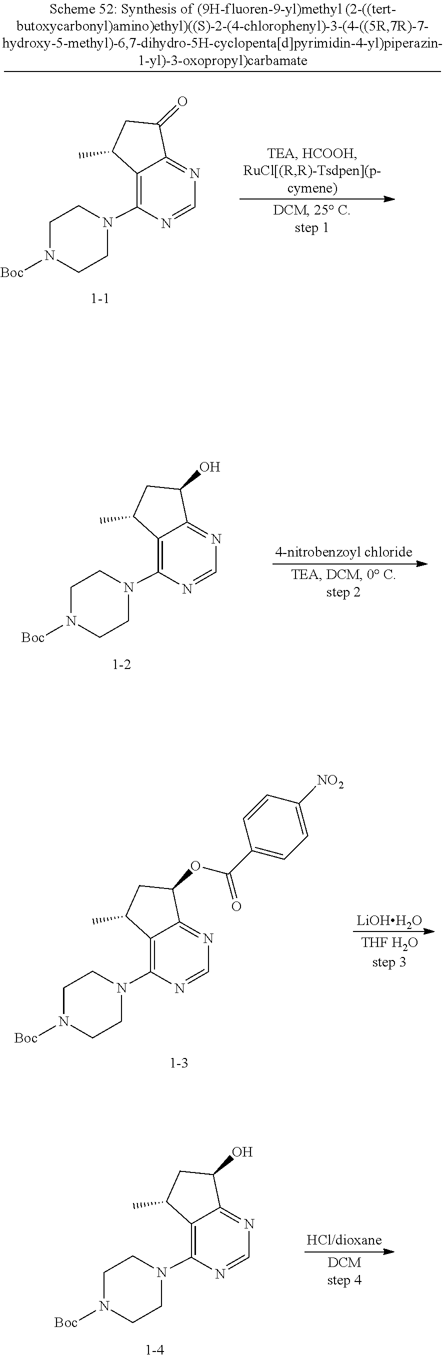 [SMILES: C[C@@H]1CC(=O)c2ncnc(N3CCN(C(=O)OC(C)(C)C)CC3)c21 ; C[C@@H]1C[C@@H](O)c2ncnc(N3CCN(C(=O)OC(C)(C)C)CC3)c21 ; C[C@@H]1C[C@@H](OC(=O)c2ccc([N+](=O)[O-])cc2)c2ncnc(N3CCN(C(=O)OC(C)(C)C)CC3)c21 ; "LiOH·H2O" ; C[C@@H]1C[C@@H](O)c2ncnc(N3CCN(C(=O)OC(C)(C)C)CC3)c21]

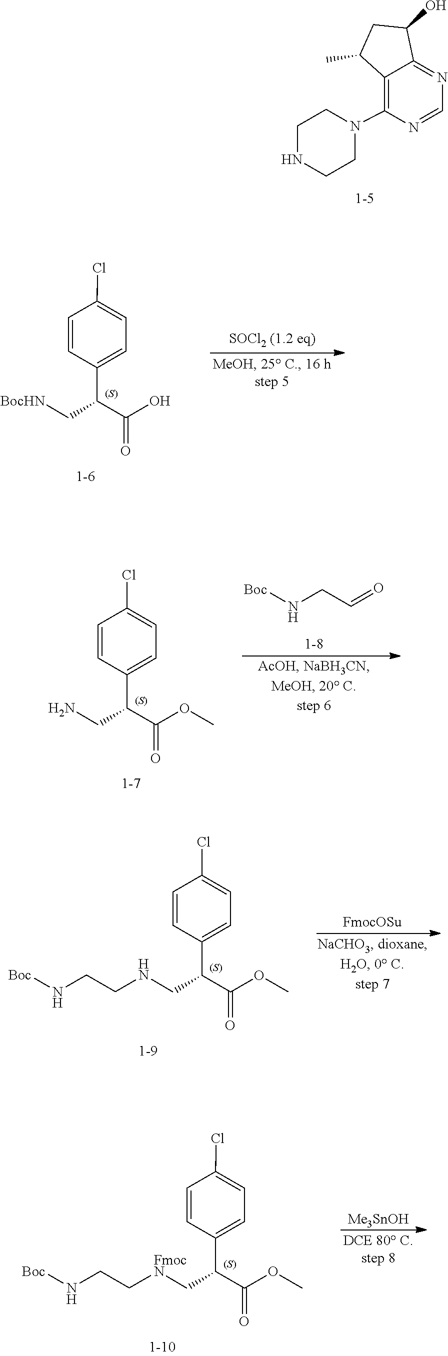

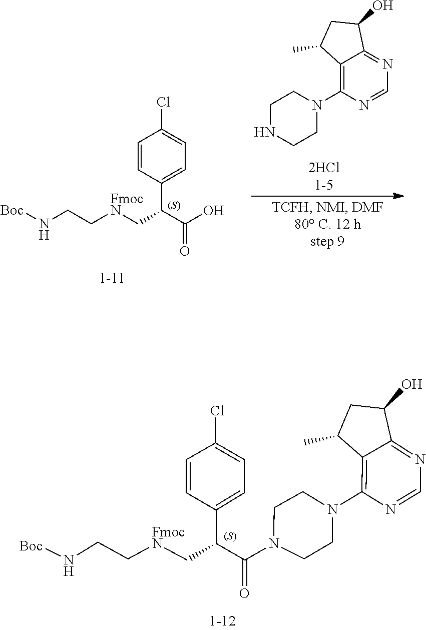

### Step 1: Synthesis of 1-2

A mixture of 1-1 (18 g, 54.15 mmol, 1 eq), TEA (5.81 g, 57.40 mmol, 7.99 mL, 1.06 eq), HCOOH (3.09 g, 64.33 mmol, 2.53 mL, 1.19 eq) in DCM (400 mL) was degassed and purged with N₂ for 3 times, and then the mixture was stirred at 25° C. for 15 min under N₂ atmosphere. Then chloro-[p-tolylsulfonyl-[rac-(1R,2R)-2-amino-1,2-diphenyl-ethyl]amino]ruthenium; 1-isopropyl-4-methyl-benzene (1.72 g, 2.71 mmol, 0.05 eq) was added in, the mixture was stirred at 25° C. for 75 min under N₂ atmosphere. Upon completion, the reaction was diluted with H₂O (200 mL), extracted with ethyl acetate 600 mL (300 mL×3). The organic phase was washed with saturated aqueous NaCl (100 mL), dried over Na₂SO₄, filtered, and concentrated under reduced pressure to give. 1-2 (18 g, crude) as a brown solid which was used in the next step without further purification. LCMS: R^{t}=0.417 min, [M+H]⁺=335.2.

### Step 2: Synthesis of 1-3

To a solution of 4-nitrobenzoyl chloride (29.96 g, 161.48 mmol, 3 eq) and 1-2 (18 g, 53.83 mmol, 1 eq) in DCM (200 mL) was added TEA (16.34 g, 161.48 mmol, 22.48 mL, 3 eq). The mixture was stirred at 0° C. for 1 hr. Upon completion, the reaction was diluted with H₂O (200 mL), extracted with ethyl acetate 600 mL (200 mL×3). The organic phase was washed with saturated aqueous NaCl (200 mL), dried over Na₂SO₄, filtered, and concentrated under reduced pressure to give a residue. The residue was purified by flash silica gel chromatography (ISCO®; 660 g SepaFlash® Silica Flash Column, Eluent of 0˜80% Ethyl acetate/Petroleum ether gradient @ 40 mL/min). The eluent was concentrated to afford product 1-3 (28 g, crude) as a brown oil. LCMS: R^{t}=0.515 min, [M+H]⁺=484.2. ¹H NMR (400 MHZ, CHLOROFORM-d) δ=8.64 (s, 1H), 8.23 (s, 4H), 6.39 (t, J=7.2 Hz, 1H), 3.92-3.77 (m, 2H), 3.75-3.58 (m, 5H), 3.56-3.45 (m, 2H), 2.48-2.26 (m, 2H), 1.50 (s, 9H), 1.29 (d, J=6.8 Hz, 3H)

### Step 3: Synthesis of 1-4

To a solution of 1-3 (23 g, 47.57 mmol, 1 eq) in THF (130 mL) and H₂O (100 mL) was added LiOH·H₂O (5.99 g, 142.70 mmol, 3 eq). The mixture was stirred at 25° C. for 1 hr. The reaction was concentrated under reduced pressure to give a residue. Then the aqueous phase was washed with saturated aqueous NaHCO₃ (20 mL), extracted with EA 60 mL (20 mL×3). The organic layers were dried over Na₂SO₄, filtered, and concentrated under reduced pressure to give 1-4 (18 g, crude) as brown oil which was used in the next step without further purification. LCMS (Method): R^{t}=0.412 min, [M+H]⁺=335.2.

### Step 4: Synthesis of 1-5

The mixture of 1-4 (18 g, 53.83 mmol, 1 eq) in 2M HCl/dioxane (90 mL) and DCM (90 mL) was stirred at 25° C. for 4 hr. The reaction was filtered, and concentrated under reduced pressure to give a residue which was purified by re-crystallization from DCM (500 mL) to give a residue of. 1-5 (10.8 g, 34.03 mmol, 63.22% yield, 96.794% purity, 2HCl) as a gray solid. LCMS: R^{t}=0.331 min, [M+H]⁺=235.2. SFC: R^{t}=2.453 min.

### Step 5: Synthesis of 1-7

To a solution of 1-6 (30 g, 100.08 mmol, 1 eq) in MeOH (300 mL) was added SOCl₂ (14.29 g, 120.10 mmol, 8.72 mL, 1.2 eq). The mixture was stirred at 25° C. for 16 hr. The reaction was filtered, and concentrated under reduced pressure to give 1-7 (20 g, 88.12 mmol, 88.05% yield, 94.14% purity) as a white solid which was used in the next step without further purification. LCMS: R^{t}=0.385 min, [M+H]⁺=214.2.

### Step 6: Synthesis of 1-9

To a solution of 1-7 (18.6 g, 87.05 mmol, 1 eq) and 1-8 (16.63 g, 104.46 mmol, 1.2 eq) in MeOH (200 mL) and AcOH (5.23 g, 87.05 mmol, 4.98 mL, 1 eq). The mixture was stirred at 20° C. for 0.5 hr. Then NaBH₃CN (10.94 g, 174.11 mmol, 2 eq) was added to the mixture. The mixture was stirred at 20° C. for 1.5 hr. The reaction was diluted with H₂O (50 mL), extracted with DCM (100 mL×3), the organic phase was washed with saturated aqueous NaCl (50 mL), dried over Na₂SO₄, filtered, and concentrated under reduced pressure to give a residue of 1-9 (40 g, crude) as a yellow oil. LCMS: R^{t}=0.458 min, [M+H]⁺=357.1.

### Step 7: Synthesis of 1-10

To a solution of 1-9 (30 g, 84.07 mmol, 1 eq) in dioxane (60 mL) and H₂O (180 mL) was added NaHCO₃ (17.66 g, 210.18 mmol, 8.18 mL, 2.5 eq) at 0° C. Then a solution of (2,5-dioxopyrrolidin-1-yl) 9H-fluoren-9-ylmethyl carbonate (34.03 g, 100.88 mmol, 1.2 eq) in dioxane (60 mL) was added to the mixture. The mixture was stirred at 0° C. for 1 hr. The reaction was diluted with H₂O (200 mL), extracted with ethyl acetate 600 mL (200 mL×3). The organic phase was washed with saturated aqueous NaCl (5 mL), dried over Na₂SO₄, filtered, and concentrated under reduced pressure to give a residue. The residue was purified by flash silica gel chromatography (ISCO®; 660 g SepaFlash® Silica Flash Column, Eluent of 0˜100% Ethyl acetate/Petroleum ether gradient @ 200 mL/min). The eluent was concentrated to afford 1-10 (28 g, 48.35 mmol, 57.51% yield, 100% purity) as a yellow oil. LCMS: Rt=0.649 min, [M+H-Boc]⁺=479.2. SFC: R^{t}=1.237 min.

### Step 8: Synthesis of 1-11

To a solution of 1-10 (10 g, 17.27 mmol, 1 eq) in DCE (100 mL) was added hydroxy (trimethyl) stannane (9.73 g, 53.81 mmol, 3.12 eq). The mixture was stirred at 80° C. for 12 hr. The reaction was cooled to 0° C., which was filtered, and concentrated under reduced pressure to give a residue. The residue was purified by flash silica gel chromatography (ISCO®; 220 g SepaFlash® Silica Flash Column, Eluent of 0˜100% Ethyl acetate/Petroleum ether gradient @ 100 mL/min). to afford product 1-11 (5.5 g, 9.52 mmol, 55.14% yield, 97.818% purity) as yellow oil. LCMS: Rt=0.632 min, [M+H-Boc]⁺=465.2. SFC: R^{t}=1.560 min.

### Step 9: Synthesis of 1-12

To a solution of 1-11 (5.5 g, 9.73 mmol, 1 eq) and 1-5 (3.59 g, 11.68 mmol, 1.2 eq, 2HCl) in DMF (55 mL) was added TCFH (5.46 g, 19.47 mmol, 2 eq) and NMI (4.00 g, 48.67 mmol, 3.88 mL, 5 eq). The mixture was stirred at 80° C. for 12 hr. The reaction was diluted with H₂O (50 mL), extracted with ethyl acetate (100 mL×3). The organic phase was washed with saturated aqueous NaCl (50 mL), dried over Na₂SO₄, filtered, and concentrated under reduced pressure to give a residue. The residue was purified by flash silica gel chromatography (ISCO®; 120 g SepaFlash® Silica Flash Column, Eluent of 0˜60% EA/MeOH gradient @ 100 mL/min). to afford 1-12 (5.5 g, 6.68 mmol, 68.59% yield, 94.85% purity) as a white solid. LCMS: R^{t}=0.539 min, [M+H]⁺=781.5; SFC: R^{t}=1.881 min, ee %=92.798%.

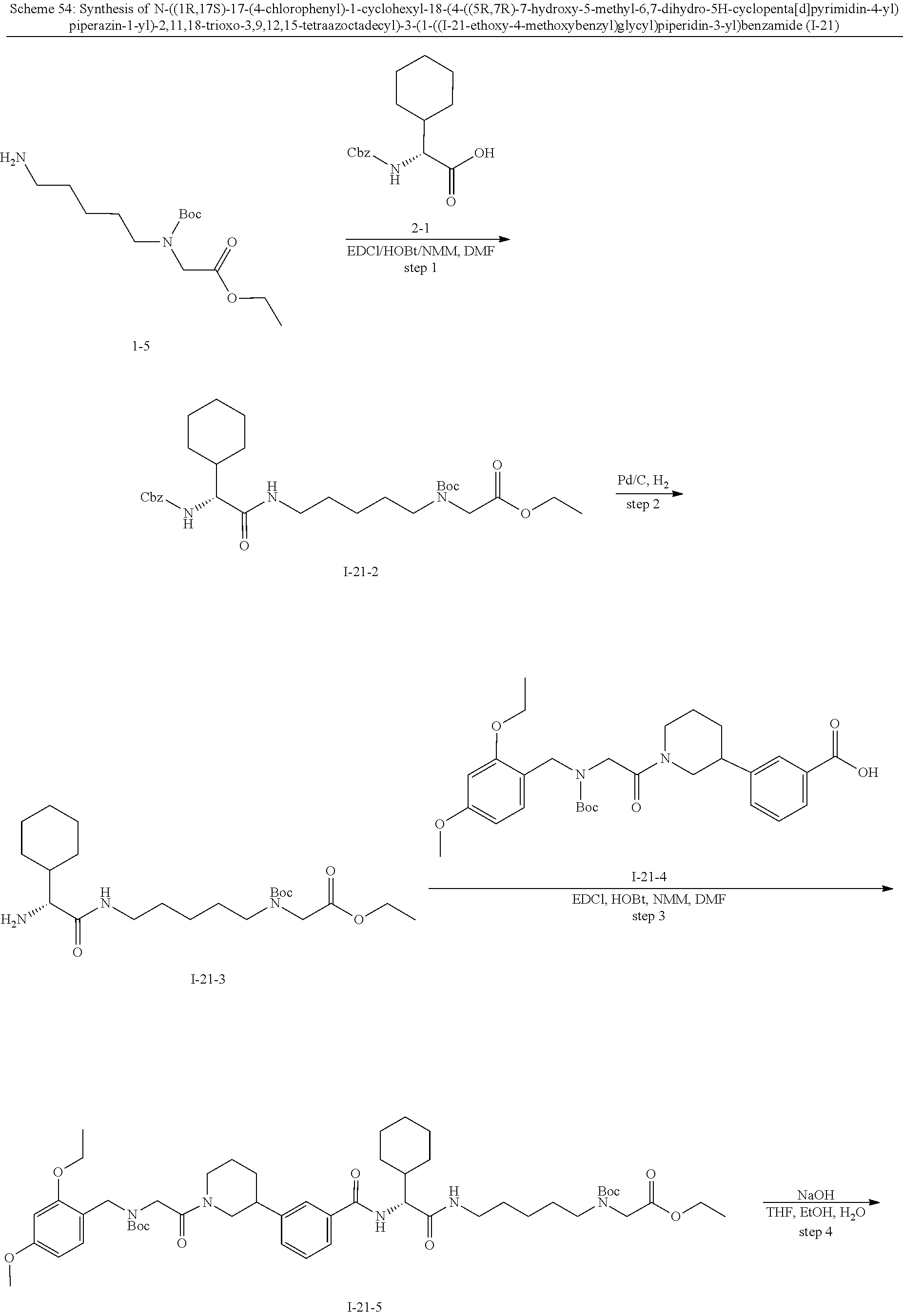

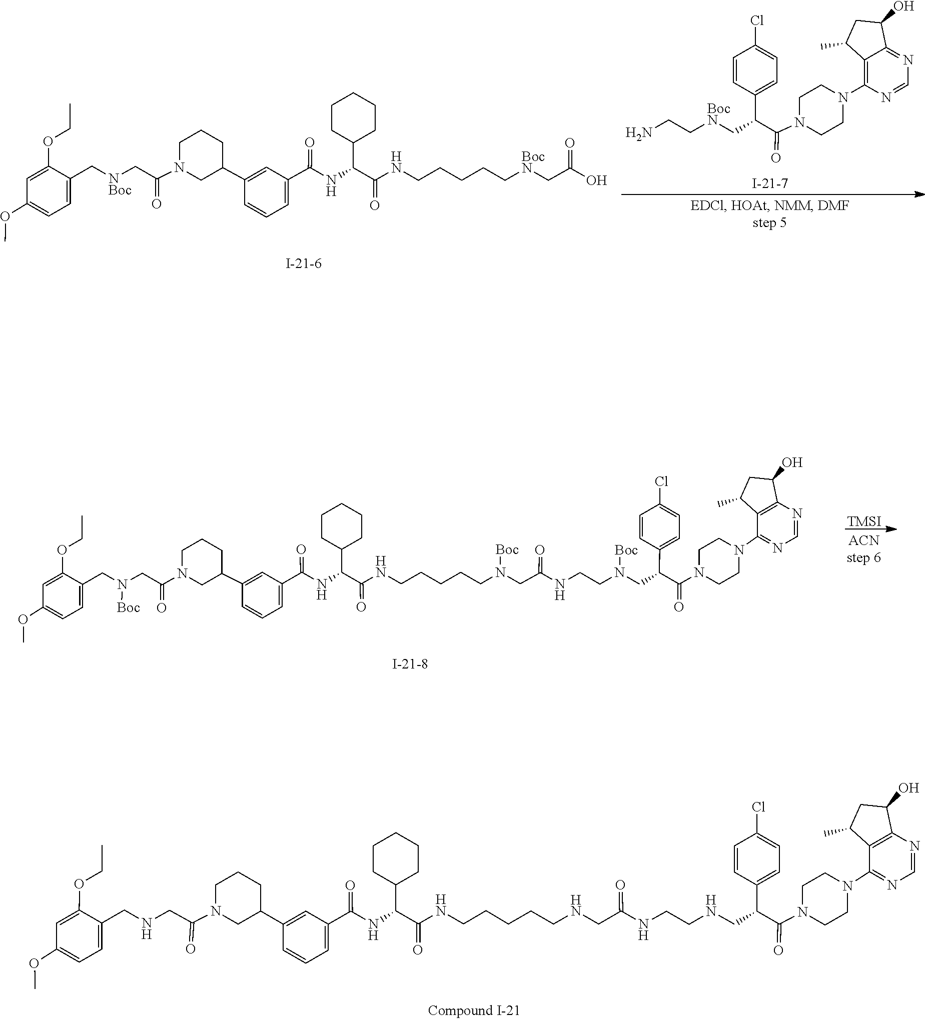

### Step 1: Synthesis of I-21-2

To a solution of ethyl 1-5 (700 mg, 2.43 mmol, 1.1 eq) and I-21-1 (642.89 mg, 2.21 mmol, 1 eq) in DMF (7 mL) was added EDCI (1.27 g, 6.62 mmol, 3 eq), HOBt (298.17 mg, 2.21 mmol, 1 eq) and NMM (2.23 g, 22.07 mmol, 2.43 mL, 10 eq). The mixture was stirred at 25° C. for 2 hr. Upon completion, the reaction mixture was concentrated under reduced pressure to give the crude product. The product was purified by prep-HPLC (column: Phenomenex luna C18 150×25 mm×10 um; mobile phase: [water (FA)-ACN]; gradient: 57%-87% B over 10 min) and the eluent was concentrated to remove MeCN and then lyophilized to give I-21-2 (600 mg, 1.04 mmol, 46.95% yield, 97% purity) as colorless oil. LCMS (Method E): R^{t}=0.622 min, [M+H]⁺=562.4, SFC: R^{t}=1.332 min.

### Step 2: Synthesis of I-21-3

To a solution of I-21-2 (600 mg, 1.07 mmol, 1 eq) in DMF (6 mL) was added Pd/C (300 mg, 281.90 μmol, 10% purity, 2.64e-1 eq), the suspension was degassed under vacuum and purged with H₂ (15 psi) three times. The mixture was stirred at 25° C. for 1 hr. Upon completion, the reaction mixture was concentrated under reduced pressure to give the crude product. The crude product I-21-3 (420 mg, 982.28 μmol, 91.96% yield, 100% purity) as obtained as a white solid. LCMS (Method E): R^{t}=0.463 min, [M+H]⁺=428.2.

### Step 3: Synthesis of I-21-5

To a solution of ethyl I-21-3 (133.97 mg, 313.32 μmol, 1.1 eq) and I-21-4 (150 mg, 284.84 μmol, 1 eq) in DMF (2 mL) was added EDCI (163.81 mg, 854.51 μmol, 3 eq), HOBt (38.49 mg, 284.84 μmol, 1 eq) and NMM (288.10 mg, 2.85 mmol, 313.15 μL, 10 eq). The mixture was stirred at 25° C. for 1 hr. Upon completion, the mixture was poured into water 10 mL and extracted with EA (10 mL×3). The combined organic layers were concentrated to give a residue. The residue was purified by flash silica gel chromatography (ISCO®; 12 g SepaFlash® Silica Flash Column, Eluent of 0˜100% Ethyl acetate/Petroleum ether gradient @ 20 mL/min) and the eluent was concentrated to give I-21-5 (250 mg, 256.36 μmol, 90.00% yield, 96% purity) as a yellow solid. LCMS (Method E): R^{t}=0.677 min, [M+H]⁺=936.5.

### Step 4: Synthesis of I-21-6

To a solution of I-21-5 (180 mg, 192.27 μmol, 1 eq) in THF (2 mL) was added NaOH (15.38 mg, 384.54 μmol, 2 eq) in H₂O (0.5 mL) and EtOH (2 mL). The mixture was stirred at 25° C. for 1 hr. Upon completion, the reaction mixture was concentrated under reduced pressure to give the crude product. The mixture was purified by reversed phased HPLC (FA) to give I-21-6 (160 mg, 169.14 μmol, 87.97% yield, 96% purity) as a white solid. LCMS (Method E): R^{t}=0.643 min, [M+H]⁺=908.7. SFC: R^{t}=0.644 min.

### Step 5: Synthesis of I-21-8

To a solution of I-21-6 (80 mg, 88.09 μmol, 1 eq) and I-21-7 (49.25 mg, 88.09 μmol, 1 eq) in DMF (1 mL) was added EDCI (50.66 mg, 264.28 μmol, 3 eq), HOAt (11.99 mg, 88.09 μmol, 1 eq) and NMM (89.10 mg, 880.93 μmol, 96.85 μL, 10 eq). The mixture was stirred at 25° C. for 1 hr. Upon completion, the mixture was poured into water 10 mL and extracted with EA (10 mL×3). The combined organic layers were concentrated to give a residue. The residue was purified by flash silica gel chromatography (ISCO®; 12 g SepaFlash® Silica Flash Column, Eluent of 0˜37% MeOH/Ethyl acetate gradient @ 20 mL/min) and the eluent was concentrated to give I-21-8 (120 mg, 76.48 μmol, 86.81% yield, 92.36% purity) was obtained as a yellow solid. LCMS (Method E): R^{t}=0.618 min, [M+H]⁺=1449.9.

### Step 6: Synthesis of I-21

To a solution of I-21-8 (50 mg, 34.50 μmol, 1 eq) in ACN (1 mL) was added TMSI (69.04 mg, 345.02 μmol, 46.96 μL, 10 eq) 0° C. The mixture was stirred at 0° C. for 0.5 hr. The reaction mixture was concentrated to give the crude product. The product was purified by prep-HPLC (column: Phenomenex luna C18 150×25 mm×10 um; mobile phase: [water (FA)-ACN]; gradient: 15%-45% B over 7 min) and the eluent was concentrated to remove MeCN and then lyophilized to give I-21 (15.78 mg, 13.21 μmol, 38.28% yield, 100% purity, FA) as colorless oil. LCMS (Method E): R^{t}=0.418 min, [M+H]⁺=1148.8. SFC: R^{t}=2.478 min and 4.582 min. ¹H NMR (400 MHZ, METHANOL-d4) δ=8.51 (s, 2H), 8.44-8.38 (m, 1H), 7.79-7.68 (m, 2H), 7.51-7.38 (m, 4H), 7.36-7.24 (m, 3H), 6.64-6.52 (m, 2H), 5.01-4.91 (m, 1H), 4.60-4.45 (m, 1H), 4.37-4.28 (m, 2H), 4.21-4.08 (m, 4H), 4.03-3.85 (m, 3H), 3.80 (d, J=4.0 Hz, 3H), 3.76-3.70 (m, 1H), 3.71 (s, 2H), 3.64-3.51 (m, 5H), 3.51-3.37 (m, 5H), 3.27-3.11 (m, 4H), 2.98-2.90 (m, 3H), 2.89-2.67 (m, 4H), 2.17-2.09 (m, 1H), 2.04-2.00 (m, 2H), 1.93-1.82 (m, 4H), 1.78 (d, J=12.8 Hz, 2H), 1.73-1.61 (m, 5H), 1.57 (d, J=6.0 Hz, 2H), 1.45-1.40 (m, 5H), 1.33-1.19 (m, 3H), 1.18-1.04 (m, 5H).

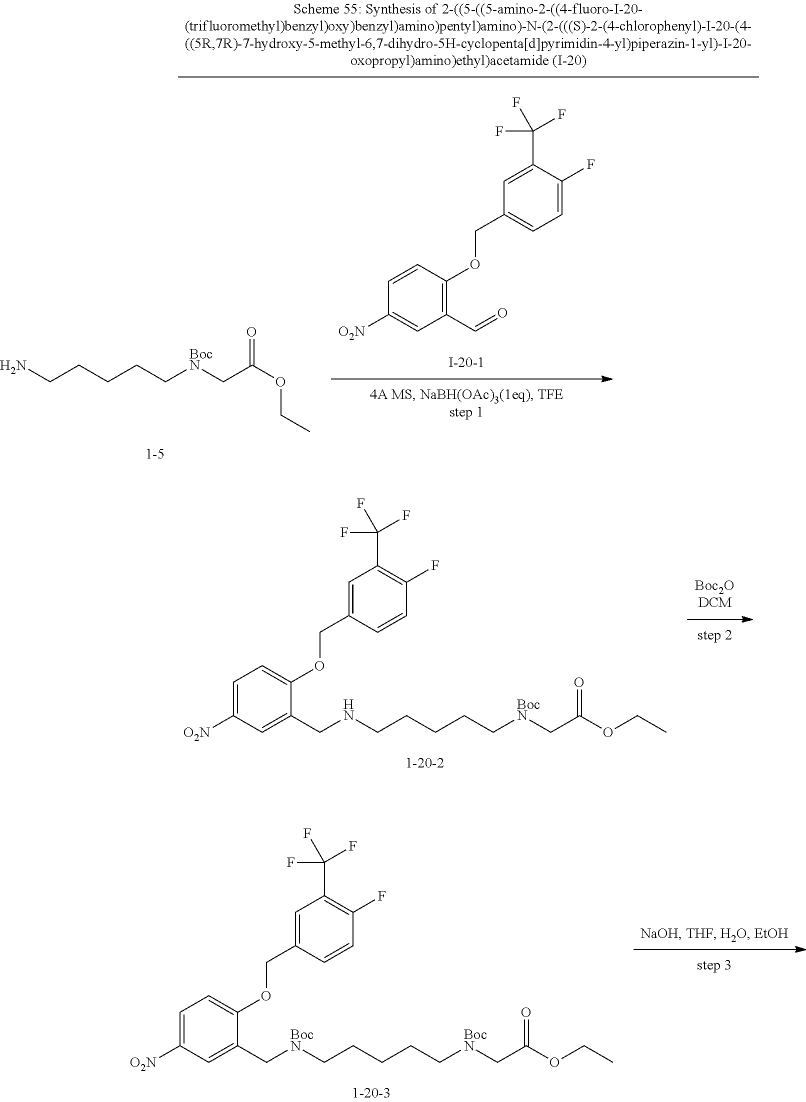 [SMILES: O=Cc1cc([N+](=O)[O-])ccc1OCc1ccc(F)c(C(F)(F)F)c1 ; CCOC(=O)CN(CCCCCN)C(=O)OC(C)(C)C ; CCOC(=O)CN(CCCCCNCc1cc([N+](=O)[O-])ccc1OCc1ccc(F)c(C(F)(F)F)c1)C(=O)OC(C)(C)C ; CCOC(=O)CN(CCCCCN(Cc1cc([N+](=O)[O-])ccc1OCc1ccc(F)c(C(F)(F)F)c1)C(=O)OC(C)(C)C)C(=O)OC(C)(C)C]

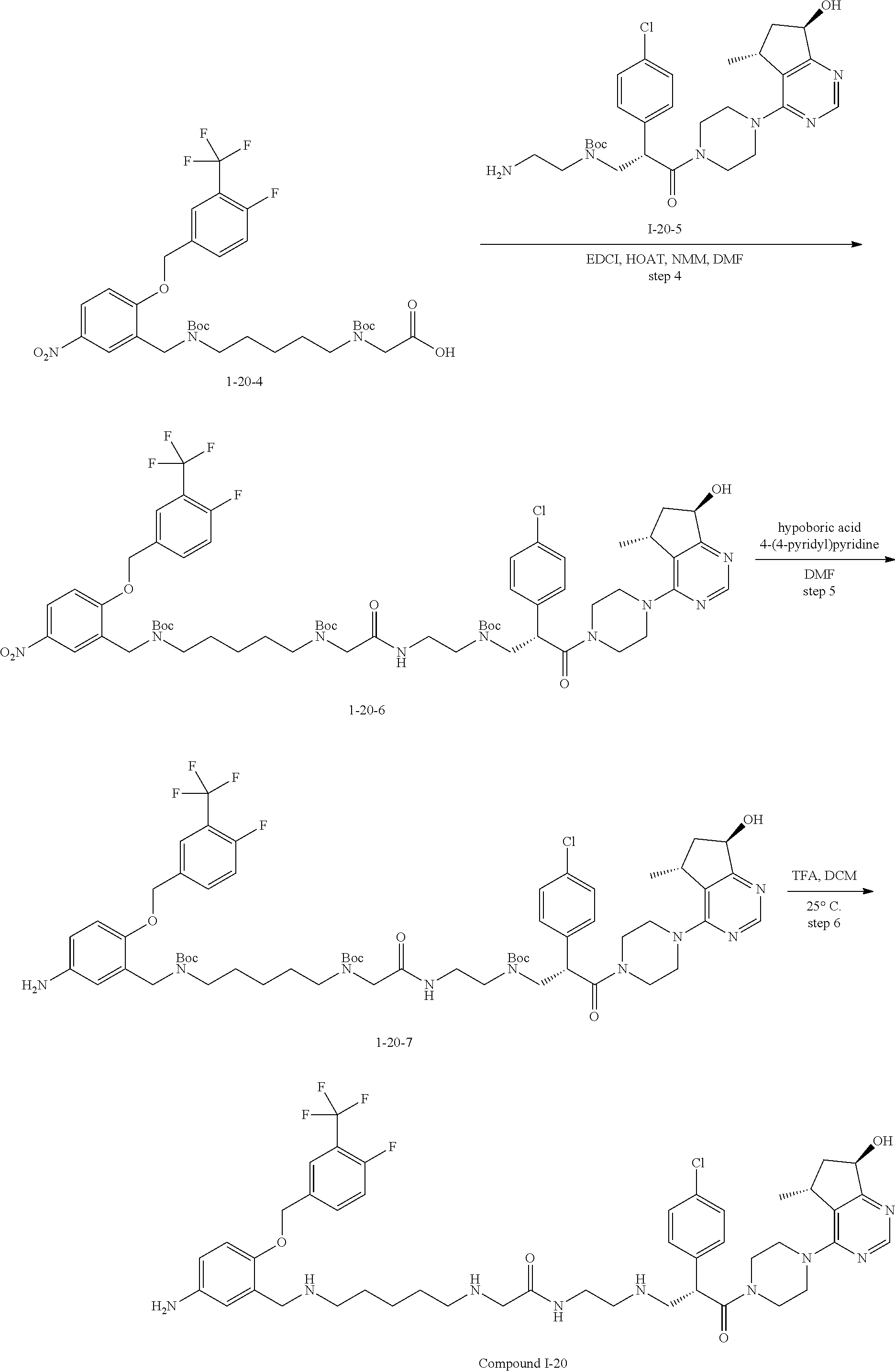

### Step 1: Synthesis of I-20-2

To a solution of 1-5 (460 mg, 1.60 mmol, 1 eq) and 4 A MS (50 mg) in TFE (5 mL) stirred at 25° C. for 0.25 h under N₂ atmosphere. Then I-20-1 (547.49 mg, 1.60 mmol, 1 eq) was added to the mixture, and the mixture was stirred at 25° C. for 0.75 h, then NaBH(OAc)₃ (338.07 mg, 1.60 mmol, 1 eq) was added to the mixture and the mixture was stirred at 25° C. for 1 hrs. Upon completion, the reaction mixture was filtered, to remove solid. The filtrate was poured into 10% K₂CO₃ aqueous (10 mL), then extracted by DCM (20 mL×3). The combined organic layer was washed with brine (15 mL), dried over anhydrous sodium sulfate, filtered, and concentrated to give residue. The residue purified by flash silica gel chromatography (ISCO®; 20 g SepaFlash® Silica Flash Column, Eluent of 0˜40% MeOH/Ethyl acetate gradient @ 40 mL/min) and the eluent was concentrated to give I-20-2 (650 mg, 633.52 μmol, 39.72% yield, 60% purity) as a yellow solid. LCMS (Method E): R^{t}=0.538 min, [M+H]⁺=616.4.

### Step 2: Synthesis of I-20-3

To a solution of I-20-2 (590 mg, 958.40 μmol, 1 eq) in DCM (6 mL) was added Boc₂O (627.50 mg, 2.88 mmol, 660.53 μL, 3 eq) and TEA (193.96 mg, 1.92 mmol, 266.79 μL, 2 eq). The mixture was stirred at 25° C. for 2 hr. Upon completion, the mixture was concentrated to give a residue. The residue was purified by flash silica gel chromatography (ISCO®; 20 g SepaFlash® Silica Flash Column, Eluent of 0˜100% Ethyl acetate/Petroleum ether gradient (40 mL/min) and the eluent was concentrated to give I-20-3 (400 mg, 525.34 μmol, 54.81% yield, 94% purity) was obtained as a white solid. LCMS (Method E): R^{t}=0.722 min, [M+Na]⁺=738.4. ¹HNMR (400 MHZ, CHLOROFORM-d) δ=8.20-8.14 (m, 1H), 8.13-7.98 (m, 1H), 7.71-7.60 (m, 2H), 7.33-7.27 (m, 1H), 6.96 (d, J=8.4 Hz, 1H), 5.19 (s, 2H), 4.55-4.37 (m, 2H), 4.22-4.16 (m, 2H), 4.12-4.00 (m, 4H), 3.97-3.75 (m, 2H), 3.23 (d, J=4.8 Hz, 4H), 2.05 (s, 5H), 1.40 (s, 9H), 1.28-1.24 (m, 9H).

### Step 3: Synthesis of I-20-4

To a solution of I-20-3 (200 mg, 279.44 μmol, 1 eq) in THF (2 mL) was added NaOH (22.35 mg, 558.87 μmol, 2 eq) in H₂O (0.6 mL) and MeOH (2 mL). The mixture was stirred at 25° C. for 1 hr. Upon completion, the reaction mixture was concentrated under reduced pressure to give the crude product. The mixture was purified by reversed phased HPLC (FA), the target peak was concentrated. Then extracted with EA (5 mL×2), dried over anhydrous sodium sulfate, filtered, and concentrated to give I-20-4 (90 mg, 126.13 μmol, 45.14% yield, 96.37% purity) as a white solid. LCMS (Method E): R^{t}=0.667 min, [M+Na]⁺=710.4.

### Step 4: Synthesis of I-20-6

To a solution of I-20-4 (81.18 mg, 118.05 μmol, 1.1 eq) and I-20-5 (60 mg, 107.32 μmol, 1 eq) in DMF (1 mL) was added EDCI (61.72 mg, 321.95 μmol, 3 eq), HOAt (14.61 mg, 107.32 μmol, 1 eq) and NMM (108.55 mg, 1.07 mmol, 117.99 μL, 10 eq). The mixture was stirred at 25° C. for 1 hr. Upon completion, the mixture was poured into water 10 mL and extracted with EA (10 mL×3). The combined organic layers were concentrated to give a residue. The residue was purified by flash silica gel chromatography (ISCO®; 12 g SepaFlash® Silica Flash Column, Eluent of 0˜37% MeOH/Ethyl acetate gradient (@ 20 mL/min) and the eluent was concentrated to give I-20-6 (120 mg, 78.13 μmol, 72.80% yield, 80% purity) as a yellow solid. LCMS (Method E): R^{t}=0.623 min, [M+H]⁺=1228.7.

### Step 5: Synthesis of I-20-7

To a solution of I-20-6 (110 mg, 89.52 μmol, 1 eq) in DMF (1.5 mL) was added 4-(4-pyridyl)pyridine (6.99 mg, 44.76 μmol, 0.5 eq). Then hypoboric acid (48.15 mg, 537.13 μmol, 6 eq) was added at 0° C. The mixture was stirred at 25° C. for 1 hr. Upon completion, the mixture was poured into water 10 mL and extracted with EA (10 mL×3). The combined organic layers were concentrated to give a residue. The residue was purified by flash silica gel chromatography (ISCO®; 12 g SepaFlash® Silica Flash Column, Eluent of 0˜37% MeOH/Ethyl acetate gradient @ 20 mL/min) and the eluent was concentrated to give I-20-7 (90 mg, 64.57 μmol, 72.12% yield, 86% purity) as a yellow solid. LCMS (Method E): R^{t}=0.555 min, [M+H]⁺=1198.8.

### Step 6: Synthesis of I-20

To a solution of I-20-7 (35 mg, 29.20 μmol, 1 eq) in DCM (0.4 mL) was added TFA (153.50 mg, 1.35 mmol, 0.1 mL, 46.11 eq). The mixture was stirred at 25° C. for 0.5 hr. Upon completion, the reaction mixture was concentrated to give a crude product. The product was purified by prep-HPLC (column: Phenomenex luna C18 150×25 mm×10 um; mobile phase: [water (FA)-ACN]; gradient: 5%-35% B over 7 min) and the eluent was concentrated to remove MeCN and then lyophilized to give I-20 (14.09 mg, 13.82 μmol, 47.34% yield, 99.31% purity, TFA) as colorless oil. LCMS (Method E): R^{t}=0.402 min, [M+H]⁺=898.4. SFC: R^{t}=2.065 min. ¹H NMR (400 MHZ, METHANOL-d4) δ=8.49-8.43 (m, 1H), 8.43-8.39 (m, 1H), 7.85-7.75 (m, 2H), 7.45-7.40 (m, 2H), 7.39-7.31 (m, 3H), 6.96 (d, J=8.4 Hz, 1H), 6.83-6.76 (m, 2H), 5.18-5.10 (m, 2H), 5.01-4.93 (m, 1H), 4.42-4.40 (m, 1H), 4.13 (s, 2H), 3.97-3.86 (m, 1H), 3.83-3.75 (m, 1H), 3.75-3.67 (m, 1H), 3.65 (s, 2H), 3.59-3.50 (m, 1H), 3.56-3.40 (m, 7H), 3.29-3.21 (m, 1H), 3.16-3.05 (m, 3H), 3.03-2.95 (m, 2H), 2.94-2.84 (m, 2H), 2.19-2.01 (m, 2H), 1.75-1.61 (m, 4H), 1.40-1.30 (m, 2H), 1.10 (d, J=6.8 Hz, 3H).

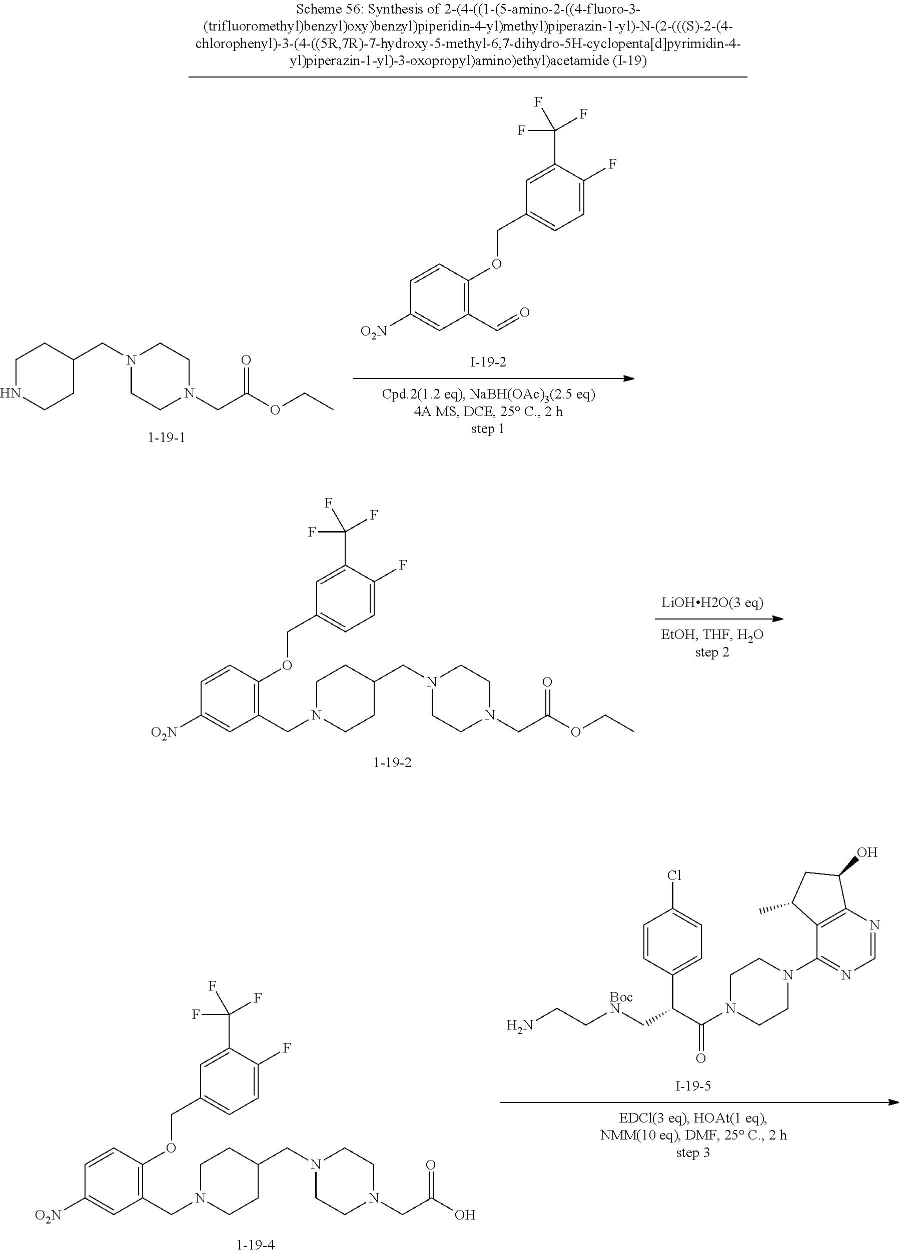 [SMILES: O=Cc1cc([N+](=O)[O-])ccc1OCc1ccc(F)c(C(F)(F)F)c1 ; CCOC(=O)CN1CCN(CC2CCNCC2)CC1 ; CCOC(=O)CN1CCN(CC2CCN(Cc3cc([N+](=O)[O-])ccc3OCc3ccc(F)c(C(F)(F)F)c3)CC2)CC1 ; O=C(O)CN1CCN(CC2CCN(Cc3cc([N+](=O)[O-])ccc3OCc3ccc(F)c(C(F)(F)F)c3)CC2)CC1 ; C[C@@H]1C[C@@H](O)c2ncnc(N3CCN(C(=O)[C@H](CN(CCN)C(=O)OC(C)(C)C)c4ccc(Cl)cc4)CC3)c21]

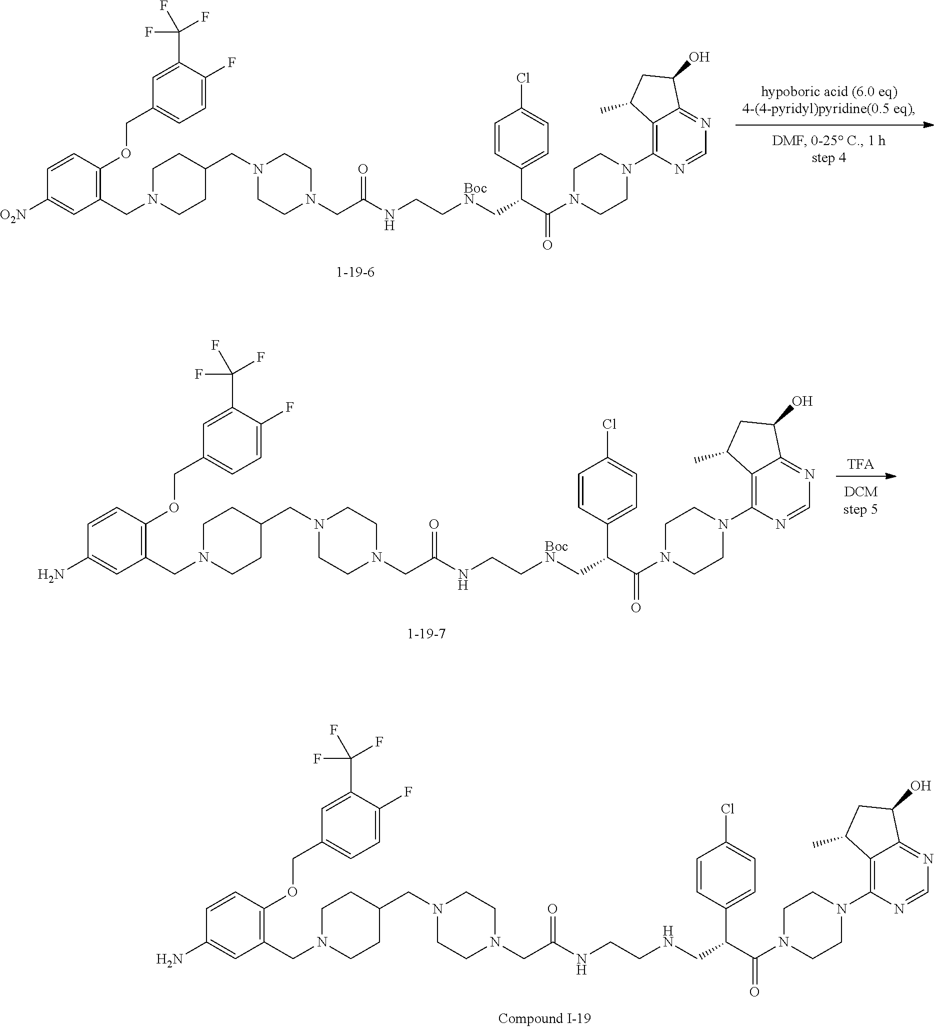 [SMILES: C[C@@H]1C[C@@H](O)c2ncnc(N3CCN(C(=O)[C@H](CN(CCNC(=O)CN4CCN(CC5CCN(Cc6cc([N+](=O)[O-])ccc6OCc6ccc(F)c(C(F)(F)F)c6)CC5)CC4)C(=O)OC(C)(C)C)c4ccc(Cl)cc4)CC3)c21 ; C[C@@H]1C[C@@H](O)c2ncnc(N3CCN(C(=O)[C@H](CN(CCNC(=O)CN4CCN(CC5CCN(Cc6cc(N)ccc6OCc6ccc(F)c(C(F)(F)F)c6)CC5)CC4)C(=O)OC(C)(C)C)c4ccc(Cl)cc4)CC3)c21 ; "TFA" ; C[C@@H]1C[C@@H](O)c2ncnc(N3CCN(C(=O)[C@H](CNCCNC(=O)CN4CCN(CC5CCN(Cc6cc(N)ccc6OCc6ccc(F)c(C(F)(F)F)c6)CC5)CC4)c4ccc(Cl)cc4)CC3)c21]

### Step 1: Synthesis of I-19-3

To a solution of I-19-1 (500 mg, 1.86 mmol, 1 eq) and 4 A MS (100 mg) in DCE (5 mL) stirred at 25° C. for 0.25 h under N₂ atmosphere. Then I-19-2 (764.48 mg, 2.23 mmol, 1.2 eq) was added to the mixture, and the mixture was stirred at 25° C. for 0.75 h, then NaBH(OAc)₃ (983.46 mg, 4.64 mmol, 2.5 eq) was added to the mixture and the mixture was stirred at 25° C. for 1 h. Upon completion, the reaction mixture was quenched by addition H₂O 5 mL, extracted with DCM 30 mL (10 mL×3). The combined organic layers were dried over Na₂SO₄, filtered, and concentrated under reduced pressure to give a residue. The residue was purified by prep-HPLC (column: Phenomenex luna C18 150×25 mm×10 um; mobile phase: [water (FA)-ACN]; gradient: 13%-33% B over 10 min), then concentrated to remove organic solvents and lyophilized to give I-19-3 (550 mg, 912.65 μmol, 49.17% yield, 99% purity) as a yellow solid. LCMS (Method E): R^{t}=0.436 min, [M+H]⁺=597.2.

### Step 2: Synthesis of I-19-4

To a solution of ethyl I-19-3 (450 mg, 754.26 μmol, 1 eq) in H₂O (1 mL), THF (4 mL), EtOH (4 mL) was added LiOH·H₂O (94.95 mg, 2.26 mmol, 3 eq). The mixture was stirred at 25° C. for 1 h. Upon completion, the reaction mixture was concentrated under reduced pressure to give a residue. The residue was purified by reversed phase (NH₃·H₂O condition), then concentrated to remove organic solvents and lyophilized to I-19-4 (300 mg, crude) as a white solid. LCMS (Method E): R^{t}=0.425 min, [M+H]⁺=569.3.

### Step 3: Synthesis of I-19-6

To a solution of I-19-4 (60 mg, 105.53 μmol, 1 eq) and I-19-6 (59.00 mg, 105.53 μmol, 1 eq) in DMF (1 mL) was added EDCI (60.69 mg, 316.59 μmol, 3 eq), HOAt (21.55 mg, 158.29 μmol, 1.5 eq) and NMM (106.74 mg, 1.06 mmol, 116.02 μL, 10 eq). The mixture was stirred at 25° C. for 2 h. Upon completion, the reaction mixture was quenched by addition H₂O 1 mL, and then diluted with EA 1 mL and extracted with EA 6 mL (2 mL×3). The combined organic layers were washed with brine 6 mL (2 mL×3), dried over anhydrous Na₂SO₄, filtered, and concentrated under reduced pressure to give a residue. The residue was purified by reversed phase (FA condition), then concentrated to remove organic solvents and lyophilized to give product I-19-6 (100 mg, 90.12 μmol, 85.40% yield) as a white solid. LCMS (Method G): R^{t}=0.837 min, [M+H]⁺=1109.8.

### Step 4: Synthesis of I-19-7

To a solution of I-19-6 (80 mg, 72.10 μmol, 1 eq) in DMF (0.8 mL) was added 4-(4-pyridyl)pyridine (5.63 mg, 36.05 μmol, 0.5 eq) and hypoboric acid (38.78 mg, 432.57 μmol, 6 eq) at 0° C. The mixture was stirred at 25° C. for 1 h. Upon completion, the reaction mixture was quenched by addition H₂O 1 mL, and then diluted with EA 1 mL and extracted with EA 6 mL (2 mL×3). The combined organic layers were washed with brine 6 mL (2 mL×3), dried over anhydrous Na₂SO₄, filtered, and concentrated under reduced pressure to give a residue. The residue was purified by reversed phase (FA condition), then concentrated to remove organic solvents and lyophilized to give I-19-7 (50 mg, 46.31 μmol, 64.24% yield) as a white solid. LCMS (Method E): R^{t}=0.445 min, [M+H]⁺=1079.7.

### Step 5: Synthesis of I-19

To a solution of I-19-7 (40 mg, 37.05 μmol, 1 eq) in DCM (1 mL) was added TFA (307.00 mg, 2.69 mmol, 200.00 μL, 72.67 eq). The mixture was stirred at 25° C. for 1 h. Upon completion, the reaction mixture was concentrated under reduced pressure to give a residue. The residue was purified by prep-HPLC (column: Phenomenex Luna C18 150×25 mm×10 um; mobile phase: [water (TFA)-ACN]; gradient: 10%-40% B over 9 min), then concentrated to remove organic solvents and lyophilized to give I-19 (39.54 mg, 36.16 μmol, 97.59% yield, 100% purity, TFA) as a white solid. LCMS (Method E): R^{t}=0.387 min, [M+H]⁺=979.5. ¹H NMR (400 MHZ, METHANOL-d4) δ=8.57 (s, 1H), 7.90-7.78 (m, 2H), 7.50-7.21 (m, 8H), 5.34-5.22 (m, 3H), 4.56-4.53 (m, 1H), 4.35 (s, 2H), 4.22-4.12 (m, 1H), 3.97-3.78 (m, 4H), 3.71-3.49 (m, 8H), 3.45-3.32 (m, 5H), 3.30-3.23 (m, 5H), 3.16-2.87 (m, 8H), 2.32-2.25 (m, 1H), 2.23-2.10 (m, 2H), 2.05 (d, J=12.4 Hz, 2H), 1.67-1.46 (m, 2H), 1.17 (d, J=7.2 Hz, 3H). SFC: R^{t}=1.180 min, ee value=100%.

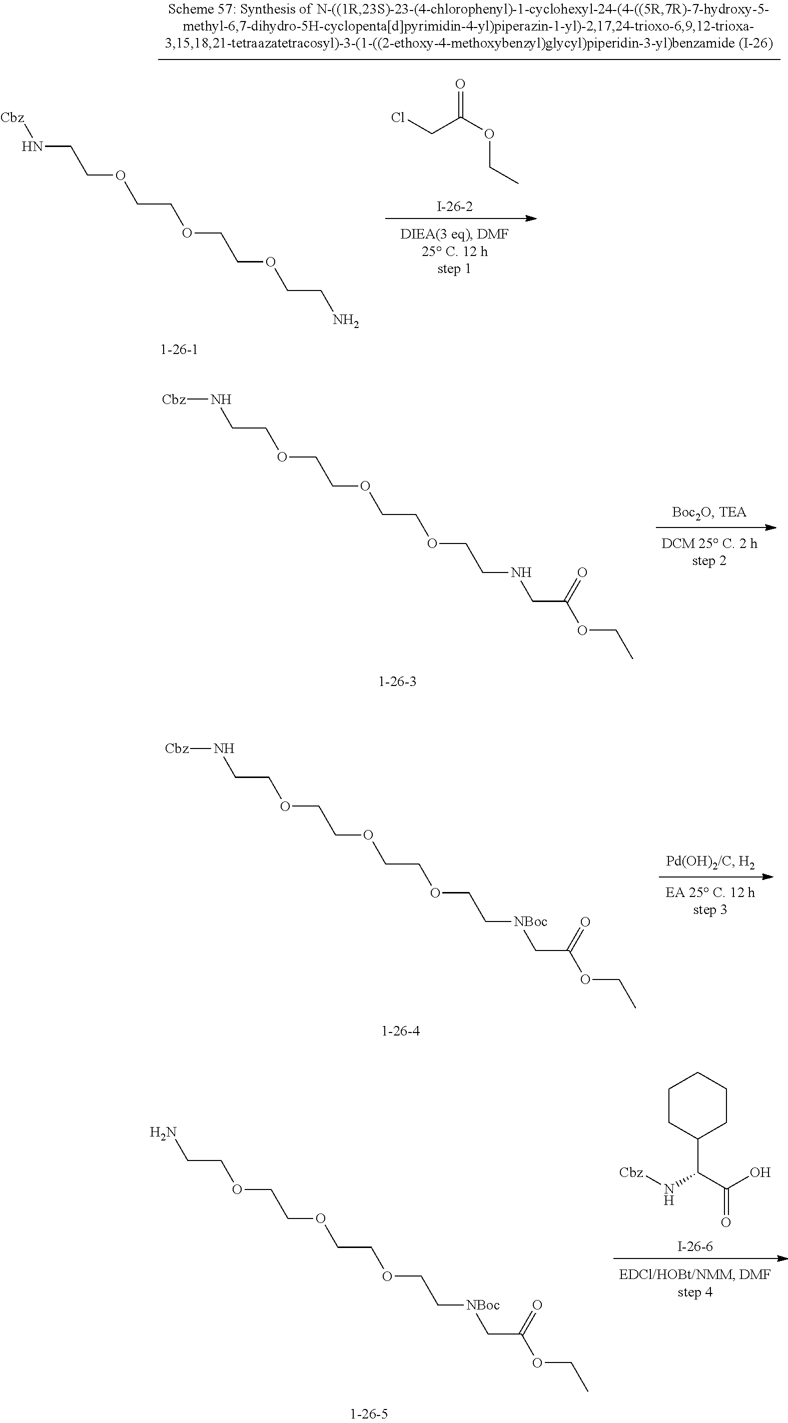

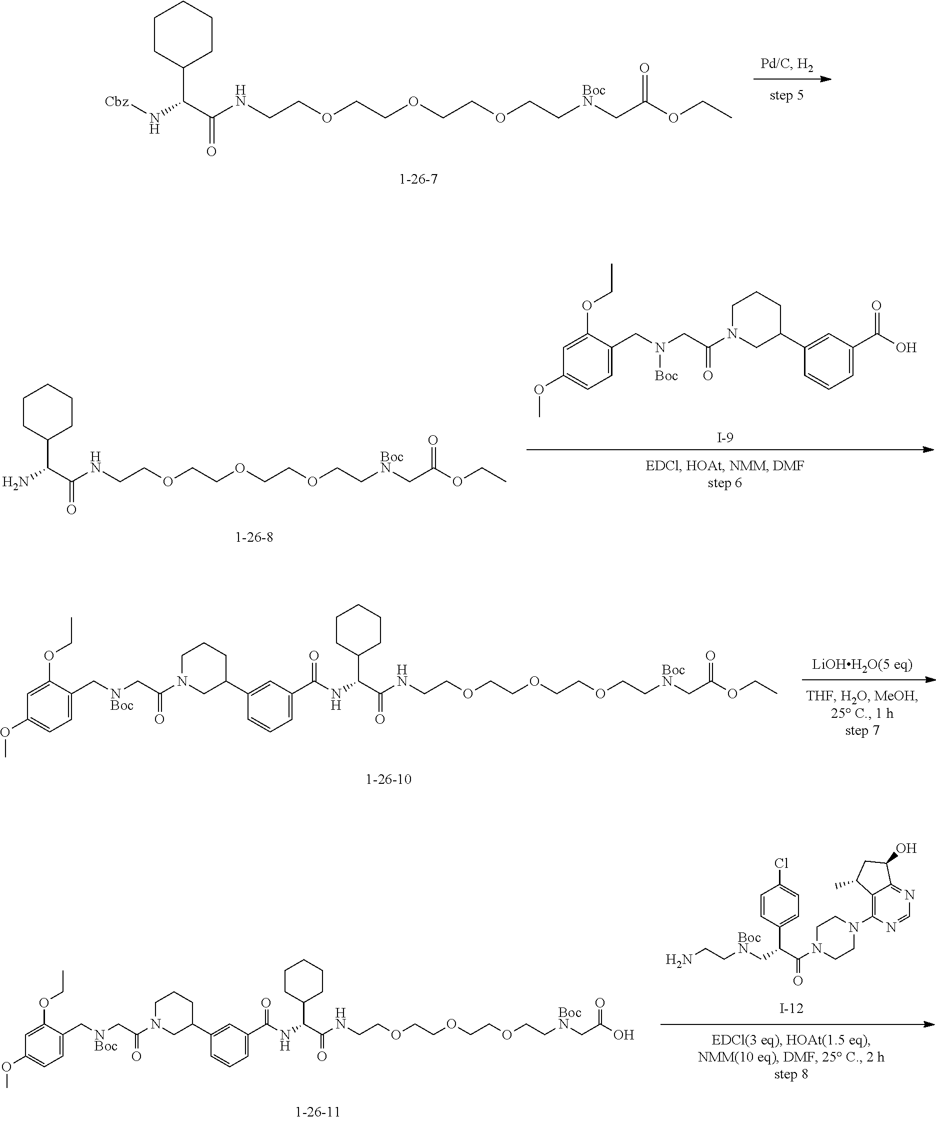

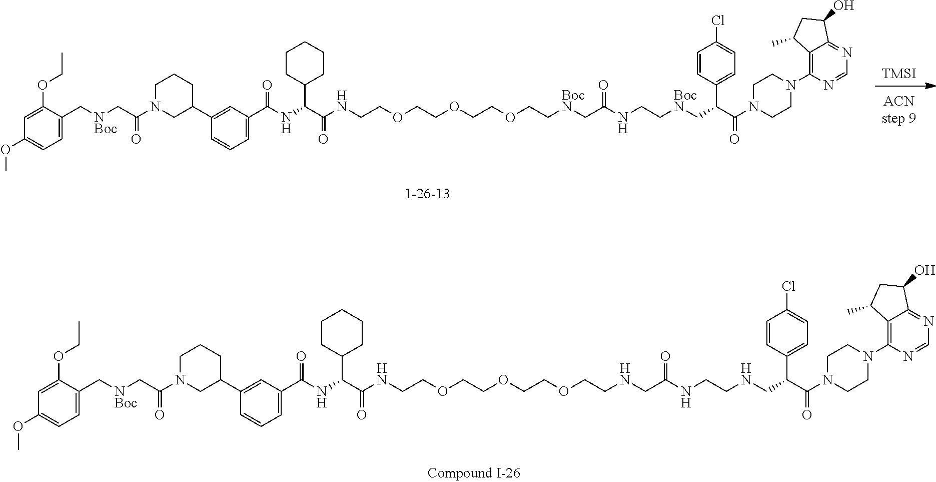 [SMILES: "TMSI" ; CCOc1cc(OC)ccc1CN(CC(=O)N1CCCC(c2cccc(C(=O)N[C@@H](C(=O)NCCOCCOCCOCCN(CC(=O)NCCN(C[C@@H](C(=O)N3CCN(c4ncnc5c4[C@H](C)C[C@H]5O)CC3)c3ccc(Cl)cc3)C(=O)OC(C)(C)C)C(=O)OC(C)(C)C)C3CCCCC3)c2)C1)C(=O)OC(C)(C)C ; CCOc1cc(OC)ccc1CN(CC(=O)N1CCCC(c2cccc(C(=O)N[C@@H](C(=O)NCCOCCOCCOCCNCC(=O)NCCNC[C@@H](C(=O)N3CCN(c4ncnc5c4[C@H](C)C[C@H]5O)CC3)c3ccc(Cl)cc3)C3CCCCC3)c2)C1)C(=O)OC(C)(C)C]

### Step 1: Synthesis of I-26-3

To a solution of I-26-1 (1 g, 3.06 mmol, 1 eq) and I-26-2 (450.57 mg, 3.68 mmol, 391.80 μL, 1.2 eq) in DMF (10 mL) was added DIEA (1.19 g, 9.19 mmol, 1.60 mL, 3 eq). The mixture was stirred at 25° C. for 12 h. Upon completion, the reaction was cooled to room temperature, which was diluted with H₂O (10 mL), extracted with ethyl acetate 60 mL (20 mL×3), The organic phase was washed with saturated aqueous NaCl (10 mL). Then dried over Na₂SO₄, filtered, and concentrated under reduced pressure to give crude product I-26-3 (1.8 g, crude) as colorless oil, and it was used into the next step without further purification. LCMS (Method G): R^{t}=0.520 min, [M+H]⁺=413.3.

### Step 2: Synthesis of I-26-4

To a solution of I-26-3 (1.7 g, 4.12 mmol, 1 eq) in DCM (10 mL) was added TEA (1.67 g, 16.49 mmol, 2.29 mL, 4 eq) and Boc₂O (2.70 g, 12.36 mmol, 2.84 mL, 3 eq). The mixture was stirred at 25° C. for 12 h. Upon completion, the reaction was filtered, and concentrated under reduced pressure to give a residue. The residue was purified by flash silica gel chromatography (ISCO®; 40 g SepaFlash® Silica Flash Column, Eluent of 0˜100% Ethyl acetate/Petroleum ether gradient @ 60 mL/min). The eluent was concentrated to afford product. I-26-4 (700 mg, 1.37 mmol, 33.13% yield) as a white solid. LCMS (Method E): R^{t}=0.557 min, [M+H]⁺=513.3. ¹H NMR (400 MHZ, CHLOROFORM-d) δ=7.40-7.28 (m, 5H), 5.35-5.34 (m, 1H), 5.11 (s, 2H), 4.23-4.14 (m, 2H), 4.08-3.95 (m, 2H), 3.66-3.53 (m, 12H), 3.51-3.45 (m, 1H), 3.45-3.33 (m, 3H), 1.45 (d, J=17.6 Hz, 9H), 1.28-1.23 (m, 3H).

### Step 3: Synthesis of I-26-5

To a solution of Pd(OH)₂/C (200 mg, 284.83 μmol, 20% purity, 2.09e-1 eq) in EtOAc (2 mL) was added I-26-4 (700 mg, 1.37 mmol, 1 eq) dissolve in EtOAc (20 mL) under N₂. The suspension was degassed under vacuum and purged with H₂ several times. The mixture was stirred under H₂ (15 psi) at 25° C. for 16 h. Upon completion, the reaction mixture was washed by MeOH 30 mL (10 mL×3) and then filtered, give I-26-5 (500 mg, 1.32 mmol, 96.74% yield) as a white oil, and it was used into the next step without further purification. LCMS (Method E): R^{t}=0.406 min, [M+H]⁺=379.3.

### Step 4: Synthesis of I-26-7

To a solution of I-26-5 (250 mg, 660.57 μmol, 1 eq) and I-26-6 (211.70 mg, 726.63 μmol, 1.1 eq) in DCM (3 mL) was added EDCI (379.90 mg, 1.98 mmol, 3 eq), DIEA (170.75 mg, 1.32 mmol, 230.12 μL, 2 eq) and HOBt (133.89 mg, 990.86 μmol, 1.5 eq). The mixture was stirred at 25° C. for 1 h. Upon completion, the reaction was filtered, and concentrated under reduced pressure to give a residue. The residue was purified by flash silica gel chromatography (ISCO®; 20 g SepaFlash® Silica Flash Column, Eluent of 0˜60% DCM/MeOH @ 40 mL/min). The eluent was concentrated to afford I-26-7 (300 mg, 420.09 μmol, 63.60% yield, 91.27% purity) as a white solid. LCMS (Method E): R^{t}=0.602 min, [M+H]⁺=652.3. SFC: R^{t}=2.134 min.

### Step 5: Synthesis of I-26-8

To a solution of Pd(OH)₂/C (100 mg, 142.42 μmol, 20% purity, 4.64 eq) in EtOAc (2 mL) was added I-26-7 (200 mg, 306.85 μmol, 1 eq) dissolve in EtOAc (10 mL) under N₂. The suspension was degassed under vacuum and purged with H₂ several times. The mixture was stirred under H₂ (15 psi) at 25° C. for 16 h. Upon completion, the reaction mixture was washed by EtOH 30 mL (10 mL×3), filtered, and concentrated under reduced pressure a residue. The crude product I-26-8 (150 mg, 289.77 μmol, 94.43% yield) was a white oil, and it was used into the next step without further purification. LCMS (Method E): R^{t}=0.461 min, [M+H]⁺=518.4.:

### Step 6: Synthesis of I-26-10

To a solution of I-26-8 (117.96 mg, 227.87 μmol, 1.2 eq) and I-26-9 (100 mg, 189.89 μmol, 1 eq) in DCM (1 mL) was added EDCI (109.21 mg, 569.6 μmol, 3 eq), DIEA (49.08 mg, 379.78 μmol, 66.15 μL, 2 eq) and HOBt (38.49 mg, 284.84 μmol, 1.5 eq). The mixture was stirred at 25° C. for 1 h. Upon completion, the reaction was diluted with H₂O (3 mL) extracted with ethyl acetate 15 mL (5 mL×3). Then dried over Na₂SO₄, filtered, and concentrated under reduced pressure to give a residue. The residue was purified by flash silica gel chromatography (ISCO®; 12 g SepaFlash® Silica Flash Column, Eluent of 0˜100% DCM/MeOH (20 mL/min). The eluent was concentrated to afford I-26-10 (250 mg, crude) as a white solid. LCMS (Method E): R^{t}=0.669 min, [M+H]⁺=1026.8.

### Step 7: Synthesis of I-26-11

To a solution of I-26-10 (100 mg, 97.44 mol, 1 eq) in H2O (0.4 mL), THF (1 mL) and MeOH (1 mL) was added LiOH·H₂O (20.44 mg, 487.21 μmol, 5 eq). The mixture was stirred at 25° C. for 1 h. Upon completion, the reaction mixture was quenched by addition H₂O 0.5 mL, and then diluted with EA 1 ml and extracted with EA 6 mL (2 mL×3). The combined organic layers were dried over anhydrous Na₂SO₄, filtered, and concentrated under reduced pressure to give I-26-11 (75 mg, 75.13 μmol, 77.11% yield) as a white solid which was used in next step without further purification. LCMS (Method E): R^{t}=0.626 min, [M+H]⁺=998.7.:

### Step 8: Synthesis of I-26-13

To a solution of I-26-11 (53.56 mg, 53.66 mol, 1 eq) and I-26-12 (30 mg, 53.66 μmol, 1 eq) in DMF (1 mL) was added EDCI (30.86 mg, 160.97 μmol, 3 eq), HOAt (10.96 mg, 80.49 μmol, 1.5 eq) and NMM (54.27 mg, 536.58 μmol, 58.99 μL, 10 eq). The mixture was stirred at 25° C. for 2 h. Upon completion, the reaction mixture was quenched by addition H₂O 1 mL at 25° C., and then diluted with EA 1 mL and extracted with EA 6 mL. The combined organic layers were washed with brine 6 mL, dried over anhydrous Na₂SO₄, filtered, and concentrated under reduced pressure to give a residue. The residue was purified by flash silica gel chromatography (ISCO®; 4 g SepaFlash® Silica Flash Column, Eluent of 0˜100% Dichloromethane: Methanol (@ 18 mL/min), then concentrated under reduced pressure to give I-26-13 (70 mg, 45.48 μmol, 84.75% yield) as a white solid. LCMS (Method E): R^{t}=0.615 min, [M+H]⁺=1540.1.:

### Step 8: Synthesis of I-26

To a solution of I-26-13 (50 mg, 32.48 μmol, 1 eq) in ACN (0.6 mL) was added TMSI (65.00 mg, 324.83 μmol, 44.21 μL, 10 eq). The mixture was stirred at 0° C. for 0.5 h. Upon completion, the reaction mixture was quenched by addition H₂O 1 mL, and then diluted with EA 1 mL and extracted with EA 6 mL. The combined organic layers were dried over anhydrous Na₂SO₄, filtered, and concentrated under reduced pressure to give a residue. The residue was purified by prep-HPLC (column: Waters Xbridge 150×25 mm×5 um; mobile phase: [water (NH4HCO3)-ACN]; gradient: 38%-68% B over 9 min), then concentrated to remove organic solvents and lyophilized to give I-26 (11.64 mg, 9.30 μmol, 28.63% yield, 98.995% purity) as a yellow solid. LCMS (Method E): R^{t}=0.452 min, [M+H]⁺=1238.9. SFC: R^{t}=3.313 min, R^{t}=6.562 min. ¹H NMR (400 MHZ, METHANOL-d4) δ=8.41 (s, 1H), 7.79-7.68 (m, 2H), 7.46-7.30 (m, 6H), 7.19-7.09 (m, 1H), 6.55-6.40 (m, 2H), 4.98-4.95 (m, 1H), 4.82-4.78 (m, 1H), 4.57-4.48 (m, 1H), 4.43-4.36 (m, 1H), 4.19-4.16 (m, 1H), 4.09-4.00 (m, 2H), 3.92-3.80 (m, 2H), 3.79-3.68 (m, 7H), 3.64-3.53 (m, 15H), 3.50-3.40 (m, 4H), 3.39-3.33 (m, 2H), 3.27-3.22 (m, 3H), 3.17-3.03 (m, 1H), 2.80-2.64 (m, 7H), 2.16-1.99 (m, 3H), 1.91-1.61 (m, 9H), 1.56-1.36 (m, 4H), 1.35-1.03 (m, 9H).

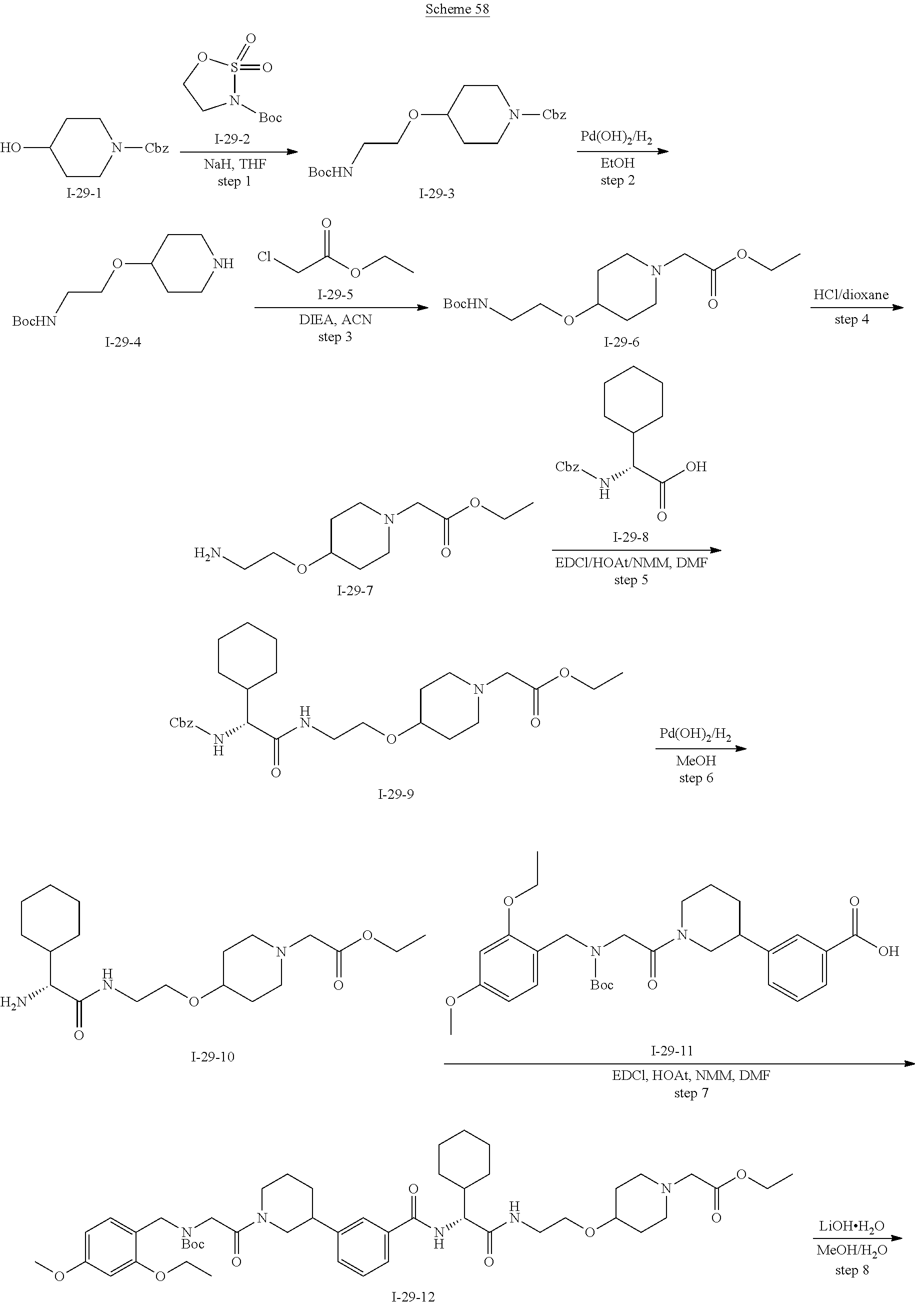

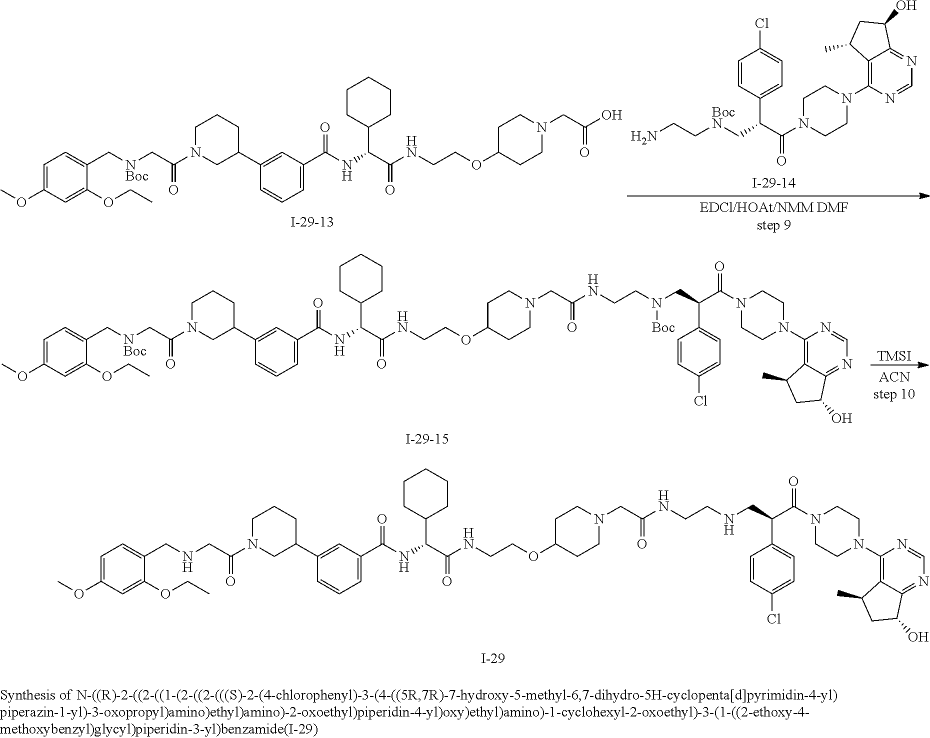

### Step 1: Synthesis of I-29-3

To a solution of I-29-1 (10 g, 42.50 mmol, 1 eq) in THF (100 mL) was added NaH (3.40 g, 85.01 mmol, 60% purity, 2 eq) and the mixture was stirred at 0° C. for 0.5 hr, then I-29-2 (9.49 g, 42.50 mmol, 1 eq) was added in. The mixture was stirred at 25° C. for 5 hr. The mixture was quenched with water (200 mL), extracted with EA (100 mL×3) and concentrated under vacuum. The residue was purified by flash silica gel chromatography (ISCO®; 120 g SepaFlash® Silica Flash Column, Eluent of 0˜50% Ethyl acetate/Petroleum ether gradient @ 80 mL/min) and concentrated under vacuum to give I-29-3 (6 g, 14.27 mmol, 33.57% yield, 90% purity) as colorless oil. LCMS (Method E): R^{t}=0.567 min, [M+H-Boc]⁺=279.3. ¹H NMR (400 MHZ, CHLOROFORM-d) δ=7.41-7.29 (m, 5H), 5.13 (s, 2H), 4.88 (s, 1H), 3.89-3.73 (m, 2H), 3.55-3.49 (m, 2H), 3.48 (s, 3H), 3.35-3.26 (m, 2H), 3.25-3.16 (m, 2H), 1.82 (s, 2H), 1.45 (s, 9H).

### Step 2: Synthesis of I-29-4

To a solution of I-29-3 (3 g, 7.93 mmol, 1 eq) in EtOH (150 mL) was added Pd(OH) 2/C (6 g, 8.54 mmol, 20% purity, 1.08 eq). The suspension was degassed and purged with H₂ for 3 times. The mixture was stirred under H₂ (50 Psi) at 25° C. for 16 hr. Upon completion, the mixture was filtered, through a pad of Celite and washed with MeOH (100 mL×5), the filtrate was concentrated under vacuum to give I-29-4 (1.54 g, 6.30 mmol, 79.51% yield, N/A purity) as colorless oil which was used into the next step without further purification. LCMS (Method E): R^{t}=0.339 min, [M+H]⁺=245.3.

### Step 3: Synthesis of I-29-6

To a solution of I-29-4 (1.4 g, 5.73 mmol, 1 eq) and I-29-5 (842.65 mg, 6.88 mmol, 732.74 μL, 1.2 eq) in ACN (15 mL) was added DIEA (3.70 g, 28.65 mmol, 4.99 mL, 5 eq). The mixture was stirred at 40° C. for 12 hr. Upon completion, the reaction was poured to water (15 mL), extracted with EA (15 mL×3) and concentrated under vacuum. The residue was purified by flash silica gel chromatography (ISCO®; 20 g SepaFlash® Silica Flash Column, Eluent of 0˜100% Ethyl acetate/Petroleum ether gradient @ 40 mL/min). I-29-6 (1.4 g, 4.24 mmol, 73.95% yield) was obtained as yellow oil. ¹H NMR (400 MHZ, CHLOROFORM-d) δ=4.89 (s, 1H), 4.21-4.16 (m, 2H), 3.50 (s, 4H), 3.39-3.15 (m, 5H), 2.88-2.71 (m, 2H), 2.42-2.31 (m, 2H), 1.91-1.89 (m, 1H), 1.73-1.64 (m, 2H), 1.51-1.43 (m, 8H), 1.33-1.25 (m, 3H).

### Step 4: Synthesis of I-29-7

A solution of I-29-6 (1.4 g, 4.24 mmol, 1 eq) in HCl/dioxane (14 mL) was stirred at 25° C. for 1 hr. Upon completion, the reaction was concentrated under vacuum. I-29-7 (1 g, 3.75 mmol, 88.47% yield, HCl) was used into the next step without further purification to get yellow solid.

### Step 5: Synthesis of I-29-9

To a solution of I-29-7 (200 mg, 868.42 μmol, 1 eq) and I-29-8 (303.61 mg, 1.04 mmol, 1.2 eq) in DMF (4 mL) was added EDCI (499.43 mg, 2.61 mmol, 3 eq), NMM (878.38 mg, 8.68 mmol, 954.76 μL, 10 eq) and HOAt (177.30 mg, 1.30 mmol, 182.22 μL, 1.5 eq). The mixture was stirred at 25° C. for 1 hr. Upon completion, the reaction was poured to water (5 mL), extracted with EA (5 mL×3). The organic layers were washed with brine (5 mL) and concentrated under vacuum. The residue was purified by flash silica gel chromatography (ISCO®; 12 g SepaFlash® Silica Flash Column, Eluent of 0˜50% Ethyl acetate/Petroleum ether gradient @ 40 mL/min). I-29-9 (400 mg, 722.75 μmol, 83.23% yield, 91% purity) was obtained as a colorless oil. LCMS (Method E): R^{t}=0.480 min, [M+H]⁺=504.2. SFC: R^{t}=1.013 min. ¹H NMR (400 MHz, METHANOL-d4) δ=7.39-7.22 (m, 6H), 5.08 (s, 2H), 4.19-4.13 (m, 2H), 3.91 (d, J=7.2 Hz, 1H), 3.55-3.45 (m, 2H), 3.43-3.35 (m, 2H), 3.21 (s, 2H), 2.78 (d, J=4.0 Hz, 2H), 2.36-2.33 (m, 2H), 1.86 (s, 2H), 1.74 (d, J=10.4 Hz, 4H), 1.69-1.57 (m, 5H), 1.30-1.16 (m, 7H), 1.12-0.99 (m, 2H).

### Step 6: Synthesis of I-29-10

To a solution of I-29-9 (600 mg, 1.19 mmol, 1 eq) in MeOH (10 mL) was added Pd(OH) 2/C (500 mg, 712.07 μmol, 20% purity, 5.98e-1 eq). The suspension was degassed and purged with H₂ for 3 times. The mixture was stirred under H₂ (15 Psi) at 25° C. for 16 hr. Upon completion, the mixture was filtered, through a pad of Celite and washed with MeOH (10 mL×5), the filter was concentrated under vacuum to give I-29-10 (400 mg, 1.08 mmol, 90.87% yield) as a yellow solid was used into the next step without further purification. ¹H NMR (400 MHZ, METHANOL-d4) δ=4.19-4.14 (m, 2H), 3.72-3.49 (m, 2H), 3.35 (s, 10H), 3.29-3.17 (m, 2H), 3.09 (d, J=5.2 Hz, 1H), 2.87-2.74 (m, 2H), 2.41-2.26 (m, 2H), 1.97-1.85 (m, 2H), 1.82-1.47 (m, 6H), 1.27-1.24 (m, 3H).

### Step 7: Synthesis of I-29-12

To a solution of I-29-11 (100.95 mg, 191.70 μmol, 1 eq) in DMF (1 mL) was added EDCI (110.25 mg, 575.11 μmol, 3 eq), HOAt (39.14 mg, 287.55 μmol, 1.5 eq) and NMM (96.95 mg, 958.51 μmol, 105.38 μL, 5 eq), then I-29-10 (85 mg, 230.04 μmol, 1.2 eq) was added in. The mixture was stirred at 25° C. for 2 hr. Upon completion, the reaction was poured to water (2 mL), extracted with EA (2 mL×3). The organic layers were washed with brine (5 mL) and concentrated under vacuum. The residue was purified by flash silica gel chromatography (ISCO®; 4 g SepaFlash® Silica Flash Column, Eluent of 0˜100% Ethyl acetate/Petroleum ether gradient @ 18 mL/min). I-29-12 (80 mg, 84.73 μmol, 44.20% yield, 93% purity) was obtained as a yellow solid. LCMS (Method E): R^{t}=0.556 min, [M+H]⁺=878.7. SFC: R^{t}=1.753 min, 2.552 min.

### Step 8: Synthesis of I-29-13

To a solution of I-29-12 (70 mg, 79.72 μmol, 1 eq) in MeOH (0.7 mL) and H₂O (0.35 mL) was added LiOH·H₂O (6.69 mg, 159.43 μmol, 2 eq). The mixture was stirred at 25° C. for 2 hr. The reaction was acidified by HCl (1M) to pH≈6 and concentrated under vacuum to give I-29-13 (65 mg, 76.47 μmol, 95.92% yield) as yellow oil which was used into the next step without further purification. LCMS (Method E): R^{t}=0.549 min, [M+H]⁺=850.4.

### Step 9: Synthesis of I-29-15

To a solution of I-29-13 (65 mg, 76.47 μmol, 1 eq) in DMF (1 mL) was added EDCI (43.98 mg, 229.40 μmol, 3 eq), HOAt (15.61 mg, 114.70 μmol, 1.5 eq) and NMM (77.35 mg, 764.66 μmol, 84.07 μL, 10 eq), then I-29-14 (42.75 mg, 76.47 μmol, 1 eq) was added in. The mixture was stirred at 25° C. for 1 hr. Upon completion, the reaction was poured to water (2 ml), extracted with EA (2 ml×3). The organic layers were washed with brine (2 ml×2) and concentrated under vacuum. The residue was purified by flash silica gel chromatography (ISCO®; 4 g SepaFlash® Silica Flash Column, Eluent of 0˜100% Ethyl acetate/Petroleum ether gradient @ 12 mL/min). I-29-15 (60 mg, 40.11 μmol, 52.46% yield, 93% purity) was obtained as yellow oil. LCMS (Method E): R^{t}=0.549 min, [M+H]⁺=1391.9. SFC: R^{t}=2.815 min, 3.517 min.

### Step 10: Synthesis of I-29

To a solution of I-29-15 (45 mg, 32.35 μmol, 1 eq) in ACN (0.9 mL) was added TMSI (64.73 mg, 323.48 μmol, 44.03 μL, 10 eq). The mixture was stirred at 0° C. for 0.5 hr. The reaction was poured to water (2 ml), basicfied by NaHCO₃ to pH≈8, extracted with CHCl₃/i-PrOH=3:1 (2 ml×3) and concentrated under vacuum. The residue was purified by prep-HPLC (FA condition column: Phenomenex luna C18 150×25 mm×10 um; mobile phase: [water (FA)-ACN]; gradient: 5%-35% B over 10 min). After lyophilization, the racemic was separated by SFC separation (column: DAICEL CHIRALPAK IK (250 mm×50 mm, 10 um); mobile phase: [CO₂-ACN/i-PrOH (0.1% NH₃H₂O)]; B %: 70%, isocratic elution mode) to give I-29 (4.93 mg, 3.81 μmol, 11.79% yield, 95.65% purity, FA) as a white solid. LCMS (Method E): R^{t}=0.421 min, [M+H]⁺=1190.9. HPLC: R^{t}=8.017 min. SFC: R^{t}=3.328 min, 5.491 min. ¹H NMR (400 MHZ, METHANOL-d4) δ=8.44 (s, 2H), 8.41 (s, 1H), 7.80-7.69 (m, 2H), 7.52-7.40 (m, 4H), 7.36-7.26 (m, 3H), 6.67-6.50 (m, 2H), 4.98-4.96 (m, 1H), 4.81-4.63 (m, 1H), 4.64-4.34 (m, 3H), 4.26-4.07 (m, 4H), 4.06-4.01 (m, 1H), 4.00-3.83 (m, 2H), 3.78-3.64 (m, 3H), 3.61 (d, J=7.2 Hz, 1H), 3.57-3.38 (m, 10H), 3.28-3.18 (m, 2H), 3.17-3.02 (m, 5H), 3.01-2.56 (m, 5H), 2.40-2.41 (m, 2H), 2.34-1.53 (m, 17H), 1.51-1.33 (m, 4H), 1.33-1.22 (m, 3H), 1.21-0.91 (m, 6H).

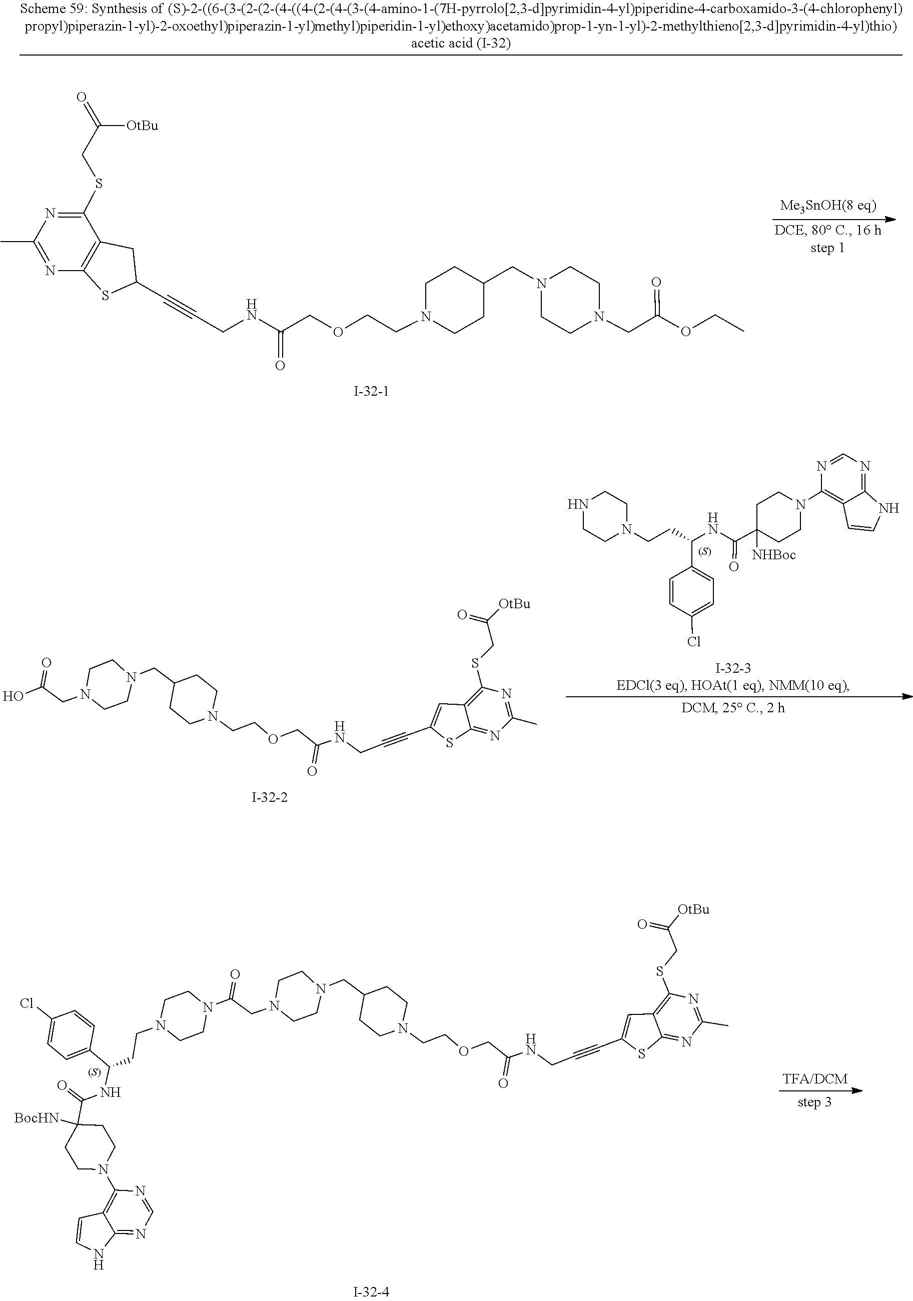

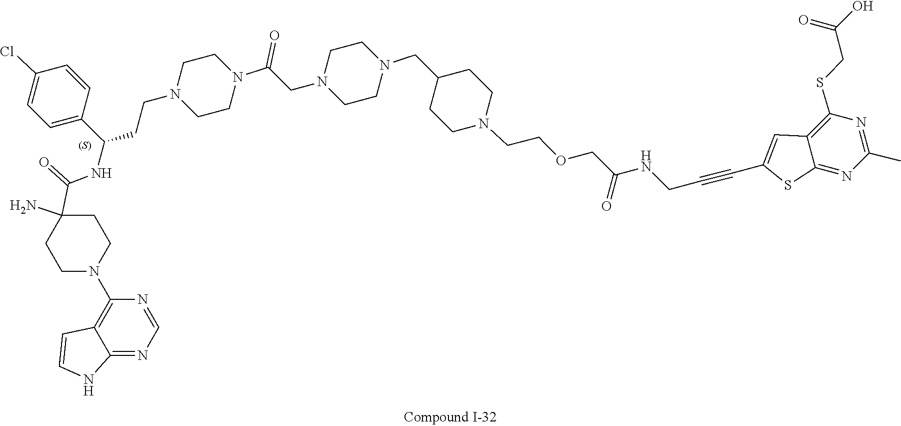

### Step 1: Synthesis of I-32-2

To a solution of I-32-2 (30 mg, 42.68 μmol, 1 eq) in DCE (0.5 mL) was added hydroxy (trimethyl) stannane (61.74 mg, 341.43 μmol, 8 eq). The mixture was stirred at 80° C. for 16 h. Upon completion, the reaction mixture was quenched by addition H₂O 1 mL, and then diluted with DCM 1 mL and extracted with DCE 3 mL (1 mL×3). The combined organic layers were dried over anhydrous Na₂SO₄, filtered, and concentrated under reduced pressure to give a residue. The residue was purified by reversed phase (FA condition), then concentrated to remove organic solvents and lyophilized to give product. I-32-2 (15 mg, 22.23 μmol, 52.08% yield) was obtained as a yellow solid. LCMS (Method G): R^{t}=0.436 min, [M+H]⁺=675.2.

### Step 2: Synthesis of I-32-4

To a solution of I-32-2 (10 mg, 14.82 μmol, 1 eq) in DCM (0.5 mL) and I-32-3 (12.17 mg, 16.30 μmol, 1.1 eq) was added EDCI (8.52 mg, 44.45 μmol, 3 eq), HOAt (2.02 mg, 14.82 μmol, 1 eq) and NMM (14.99 mg, 148.18 μmol, 16.29 μL, 10 eq). The mixture was stirred at 25° C. for 2 h. Upon completion, the reaction mixture was quenched by addition H₂O 0.5 mL, and then diluted with DCM 0.5 mL and extracted with DCM 3 mL (1 mL×3). The combined organic layers were dried over anhydrous Na₂SO₄, filtered, and concentrated under reduced pressure to give a residue and used in next step without further purification. I-32-4 (10 mg, 7.97 μmol, 53.82% yield) was obtained as a yellow solid. LCMS (Method G): R^{t}=0.843 min, [M+H]⁺=1253.8.

### Step 3: Synthesis of I-32

To a solution of I-32-4 (10 mg, 7.97 μmol, 1 eq) in DCM (0.3 mL) was added TFA (153.50 mg, 1.35 mmol, 0.1 mL, 168.82 eq). The mixture was stirred at 25° C. for 2 h. Upon completion, the reaction mixture was concentrated under reduced pressure to give a residue. The residue was purified by prep-HPLC (column: Waters Xbridge 150×25 mm×5 um; mobile phase: [water (ammonia hydroxide v/v)-ACN]; gradient: 10%-40% B over min), then concentrated to remove organic solvents and lyophilized to give I-32 (3.4 mg, 3.10 μmol, 38.84% yield, 100% purity) as a white solid. LCMS (Method G): R^{t}=0.419 min, [M+H]⁺=1098.5. SFC: R^{t}=0.655 min. ¹H NMR (400 MHZ, METHANOL-d4) δ=8.12 (s, 1H), 7.38 (s, 1H), 7.32 (s, 4H), 7.11 (d, J=3.6 Hz, 1H), 6.61 (d, J=3.6 Hz, 1H), 4.97 (s, 1H), 4.53-4.45 (m, 2H), 4.36 (s, 2H), 4.10 (d, J=5.2 Hz, 4H), 3.84-3.81 (m, 2H), 3.69-3.57 (m, 6H), 3.50-3.43 (m, 2H), 3.22 (s, 2H), 3.20-3.16 (m, 2H), 2.81-2.70 (m, 2H), 2.64 (s, 3H), 2.57-2.42 (m, 10H), 2.40-2.32 (m, 3H), 2.26-2.13 (m, 4H), 2.09-1.97 (m, 2H), 1.97-1.90 (m, 2H), 1.82-1.73 (m, 1H), 1.65-1.57 (m, 2H), 1.44 (s, 2H), 1.32-1.28 (m, 1H).

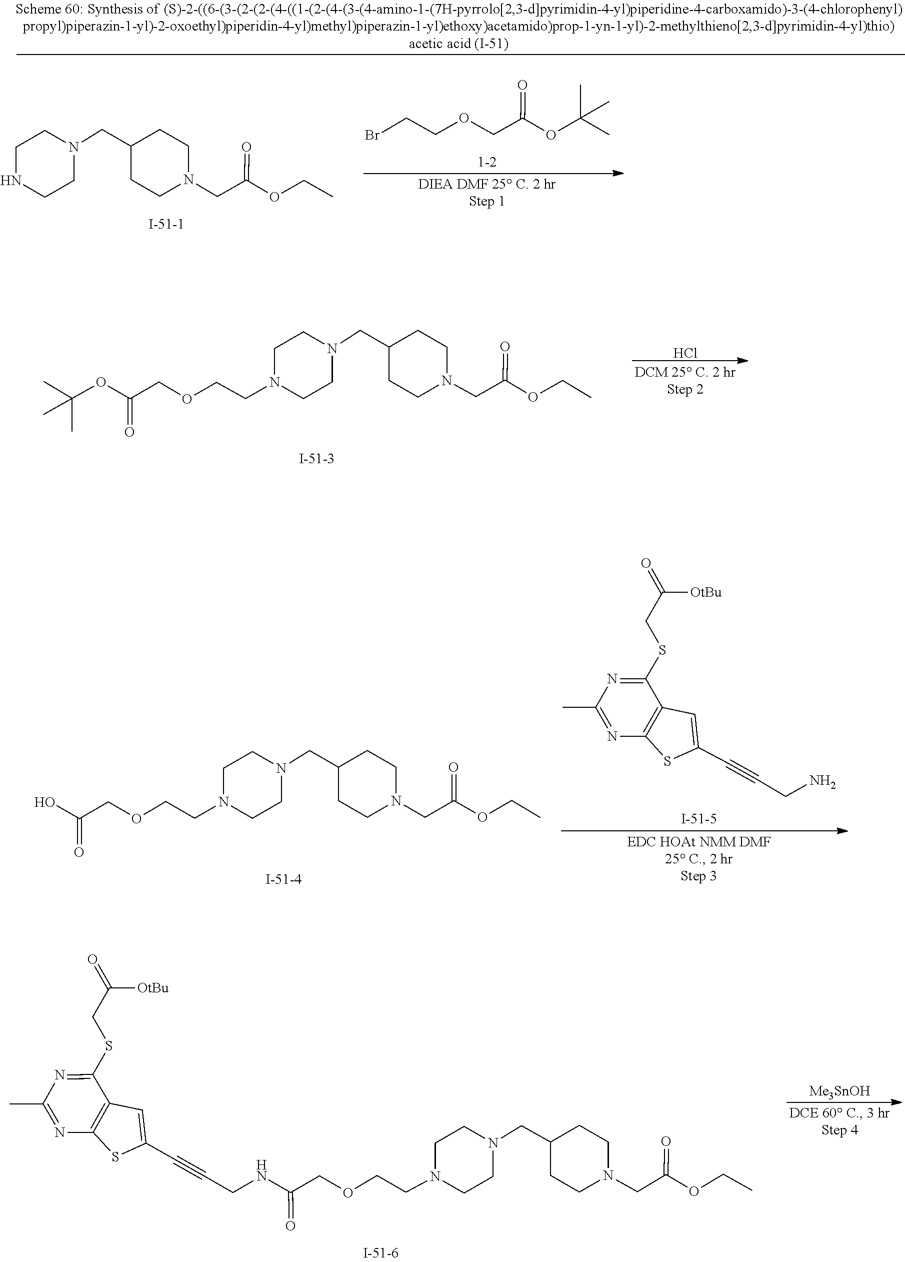

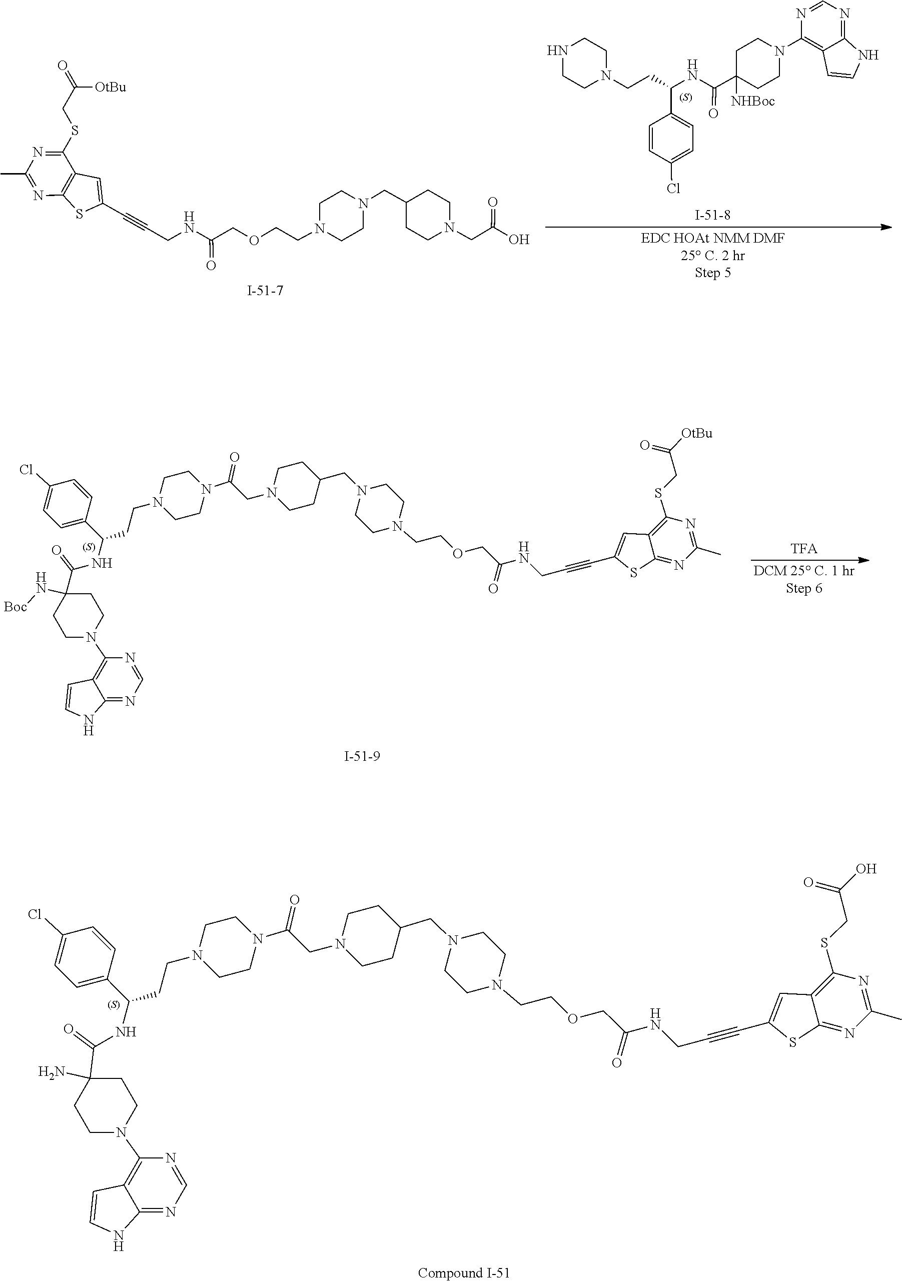

### Step 1: Synthesis of I-51-3

To a solution of I-51-1 (340 mg, 1.11 mmol, 1.2 eq, HCl) and I-51-2 (886.03 mg, 3.71 mmol, 4 eq) in DMF (4 mL) was added DIEA (598.65 mg, 4.63 mmol, 806.81 μL, 5 eq). The mixture was stirred at 25° C. for 2 hr. Upon completion, the mixture poured into water 10 mL, then was added in citric acid (20 mg) to PH<6 and extracted with EA (10 mL×3). Then the aqueous phase was added in Na₂CO₃ (10 mg) to PH>8 and extracted with EA (10 mL×3), the combined organic layers were concentrated to give I-51-3 (120 mg, 244.17 μmol, 26.36% yield, 87% purity) as a white solid. LCMS (Method E): R^{t}=0.355 min, [M+H]⁺=428.3, 1H NMR (400 MHZ, DMSO-d₆) δ=3.95 (s, 2H), 3.53-3.50 (m, 2H), 3.20-3.11 (m, 2H), 2.78 (d, J=11.2 Hz, 2H), 2.45-2.40 (m, 2H), 2.42-2.21 (m, 6H), 2.16-2.04 (m, 4H), 1.66-1.56 (m, 2H), 1.42 (s, 9H), 1.18-1.15 (m, 5H).

### Step 2: Synthesis of I-51-4

To a solution of I-51-3 (80 mg, 187.10 μmol, 1 eq) in DCM (0.8 mL) was added HCl (12 M, 77.96 μL, 5 eq). The mixture was stirred at 25° C. for 2 hr. Upon completion, the reaction mixture was concentrated under reduced pressure to give I-51-4 (66 mg, 177.67 μmol, 94.96% yield) as a yellow solid. LCMS (Method E): R^{t}=0.120 min, [M+H]⁺=372.4. ¹H NMR (400 MHZ, METHANOL-d4) δ=4.33 (m, 2H), 4.26 (s, 1H), 4.22 (s, 2H), 4.20-4.16 (m, 2H), 4.12 (s, 1H), 4.00-3.93 (m, 5H), 3.86 (s, 1H), 3.78-3.70 (m, 4H), 3.60 (s, 3H), 3.35-3.33 (d, J=8.4 Hz, 2H), 3.21 (m, 2H), 2.38-2.25 (m, 3H), 1.77-1.67 (m, 2H), 1.33 (m, 3H).

### Step 3: Synthesis of I-51-6

To a solution of I-51-4 (200 mg, 538.40 μmol, 1 eq) in DMF (2 mL) was added I-51-5 (206.97 mg, 592.24 μmol, 1.1 eq), EDCI (206.43 mg, 1.08 mmol, 2 eq), HOBt (72.75 mg, 538.40 μmol, 1 eq) and NMM (272.29 mg, 2.69 mmol, 295.97 μL, 5 eq). The mixture was stirred at 25° C. for 2 hr. Upon completion, the reaction mixture was quenched by addition water 20 mL at 25° C., and then extracted with EA 15 mL (5 mL×3). Then dried over Na₂SO₄, filtered, and concentrated under reduced pressure to give a residue. The residue was purified by flash silica gel chromatography (ISCO®; 40 g SepaFlash® Silica Flash Column, Eluent of 0˜50% Ethyl acetate/Methanol @ 60 mL/min). I-51-6 (300 mg, 354.23 μmol, 65.79% yield, 83% purity) as a yellow solid. LCMS (Method E): R^{t}=0.458 min, [M+H]⁺=703.4.

### Step 4: Synthesis of I-51-7

To a solution of I-51-6 (150 mg, 213.39 μmol, 1 eq) in DCE (2 mL) was added hydroxy (trimethyl) stannane (192.93 mg, 1.07 mmol, 5 eq). The mixture was stirred at 60° C. for 3 hr. Upon completion, the reaction mixture was quenched by addition water 5 mL at 25° C., and then extracted with EA 15 mL. Then dried over Na₂SO₄, filtered, and concentrated under reduced pressure to give a residue. The residue was purified by prep-HPLC (column: Waters Xbridge 150×25 mm×5 um; mobile phase: [water (ammonia hydroxide v/v)-ACN]; gradient: 18%-48% B over min), the target peak was concentrated and lyophilized to afford I-51-7 (60 mg, 86.24 μmol, 40.41% yield, 97% purity) as a yellow solid. LCMS: R^{t}=0.492 min, [M−H]⁻=673.2.

### Step 5: Synthesis of I-51-9

To a solution of I-51-7 (40 mg, 59.27 μmol, 1 eq) in DMF (0.5 mL) was added HOAt (8.07 mg, 59.27 μmol, 1 eq), EDCI (45.45 mg, 237.08 mol, 4 eq), NMM (59.95 mg, 592.70 mol, 65.16μ, 10 eq) and I-51-8 (42.47 mg, 71.12 μmol, 1.2 eq). The mixture was stirred at 25° C. for 2 hr. Upon completion, the reaction mixture was quenched by addition water 5 mL at 25° C., and then extracted with EA 15 mL (5 mL×3). Then dried over Na₂SO₄, filtered, and concentrated under reduced pressure to give a residue. The residue was purified by flash silica gel chromatography (ISCO®; 20 g SepaFlash® Silica Flash Column, Eluent of 0˜40% Menthol/Ethyl acetate @ 40 mL/min). I-51-9 (55 mg, 34.65 μmol, 58.46% yield, 79% purity) was obtained as a yellow solid. LCMS (Method E): R^{t}=0.458 min, [M+H]⁺=1254.6.

### Step 6: Synthesis of I-51

To a solution of t I-51-9 (35 mg, 27.91 μmol, 1 eq) in DCM (0.5 mL) was added TFA (153.50 mg, 1.35 mmol, 0.1 mL, 48.23 eq). The mixture was stirred at 25° C. for 1 hr. The reaction mixture was concentrated under reduced pressure to give a residue. The residue was purified by prep-HPLC (column: Phenomenex luna C18 150×25 mm×10 um; mobile phase: [water (TFA)-ACN]; gradient: 10%-40% B over min), the target peak was concentrated and lyophilized to afford I-51 (22 mg, 18.08 μmol, 64.77% yield, 99.57% purity, TFA) as yellow oil. LCMS (Method E): R^{t}=0.395 min, [M+H]⁺=1097.5. SFC: R^{t}=1.330 min, ee %=100%. ¹H NMR (400 MHZ, METHANOL-d4) δ=8.34 (s, 1H), 7.42 (s, 1H), 7.36-7.22 (m, 5H), 6.89-6.88 (d, J=3.6 Hz, 1H), 4.99 (m, 2H), 4.57-4.54 (d, J=14.0 Hz, 3H), 4.30 (s, 2H), 4.25 (s, 2H), 4.13-4.11 (d, J=5.6 Hz, 4H), 3.92-3.78 (m, 5H), 3.76-3.55 (m, 4H), 3.37-3.36 (d, J=4.8 Hz, 8H), 3.28-3.27 (d, J=1.6 Hz, 5H), 3.19-3.08 (m, 2H), 3.02-3.01 (d, J=4.8 Hz, 2H), 2.90-2.86 (d, J=15.6 Hz, 4H), 2.63 (s, 4H), 2.59-2.51 (m, 1H), 2.46-2.44 (d, J=4.8 Hz, 2H), 2.36-2.34 (d, J=10.4 Hz, 1H), 2.29-2.21 (m, 1H), 2.19-2.12 (m, 1H), 2.06-1.87 (m, 4H), 1.53 (d, J=2.4 Hz, 2H), 1.33-1.21 (m, 1H).

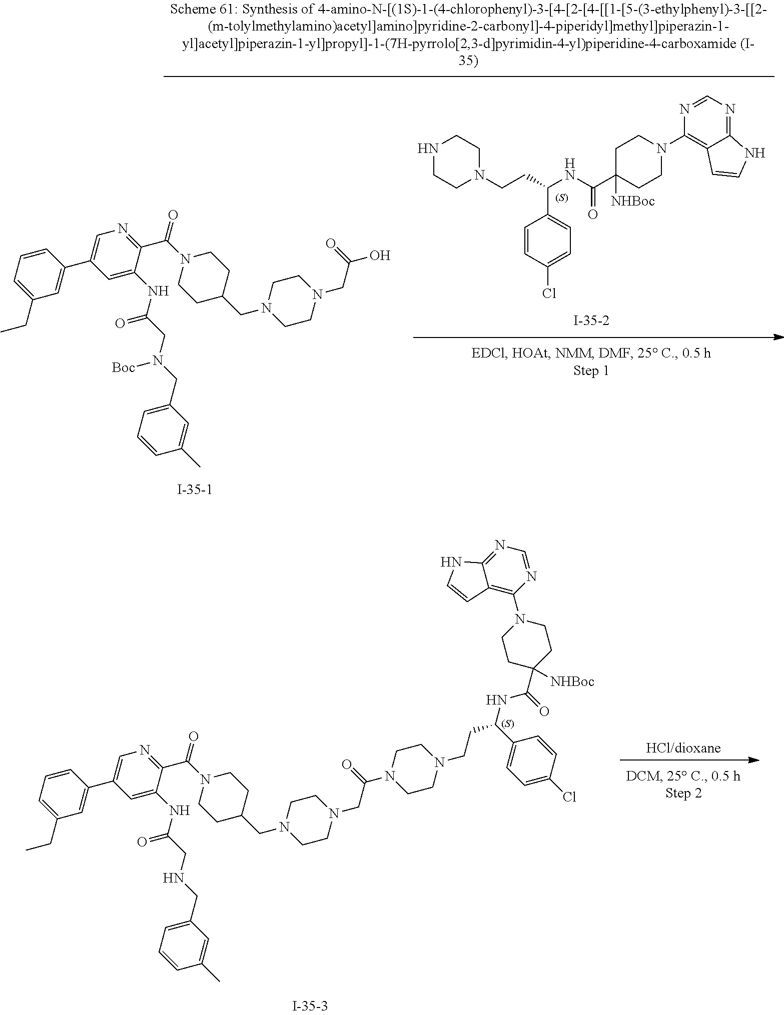

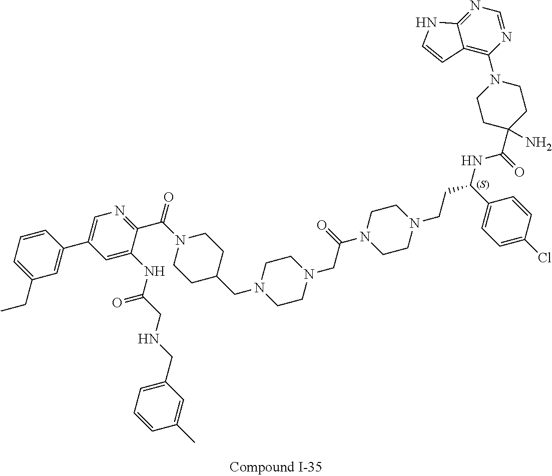

### Step 1: Synthesis of I-35-3

To a solution of I-35-2 (40 mg, 66.98 μmol, 1 eq) and I-35-1 (53.56 mg, 73.68 μmol, 1.1 eq) in DMF (0.5 mL) was added EDCI (38.52 mg, 200.95 μmol, 3 eq) and HOAt (9.12 mg, 66.98 μmol, 9.37 μL. 1 eq), NMM (33.88 mg, 334.92 μmol, 36.82 μL, 5 eq). The mixture was stirred at 25° C. for 0.5 hr. Upon completion, the mixture was diluted with water (5 mL) and extracted with DCM (5 mL×3). The combined organic layer was dried over Na₂SO₄, filtered, and concentrated in vacuo to give a residue. The residue was purified by prep-HPLC (column: Phenomenex luna C18 150×25 mm×10 um; mobile phase: [water (FA)—ACN]; gradient: 24%-54% B over 10 min). Then eluent was concentrated in vacuum and lyophilized. I-35-3 (40 mg, 33.17 μmol, 49.52% yield) was obtained as a white solid. LCMS (Method D): R^{t}=0.410 min, [(M+H)/2]⁺=603.8.

### Step 2: Synthesis of I-35

To a solution of I-35-3 (40 mg, 33.17 μmol, 1 eq) in DCM (0.5 mL) was added HCl/dioxane (4 M, 0.2 mL, 24.12 eq). The mixture was stirred at 25° C. for 0.5 hr. Upon completion, the reaction mixture was concentrated in vacuum to give a residue. The residue was used into next step directly without further purification. I-35 (37.9 mg, 31.72 μmol, 95.63% yield, 95.598% purity, HCl) was obtained as a white solid. LCMS (Method D): R^{t}=0.317 min, [M+H]⁺=1105.5. SFC Data: R^{t}=0.654 min. ¹H NMR (400 MHZ, METHANOL-d4) δ=8.69 (s, 1H), 8.40 (s, 1H), 8.33 (s, 1H), 7.55-7.47 (m, 2H), 7.47-7.40 (m, 4H), 7.38 (d, J=5.6 Hz, 3H), 7.36-7.28 (m, 4H), 7.02 (d, J=3.2 Hz, 1H), 5.05-5.03 (m, 1H), 4.72-4.58 (m, 4H), 4.47-4.35 (m, 1H), 4.29 (s, 2H), 4.15-4.06 (m, 1H), 4.03 (s, 2H), 3.98-3.90 (m, 2H), 3.89-3.80 (m, 2H), 3.74-3.53 (m, 10H), 3.36-3.34 (m, 2H), 3.25 (s, 6H), 2.98 (t, J=12.8 Hz, 1H), 2.89-2.80 (m, 1H), 2.75-2.72 (m, 3H), 2.56-2.47 (m, 1H), 2.40 (s, 3H), 2.34-2.25 (m, 2H), 2.22 (s, 1H), 2.06 (d, J=13.6 Hz, 2H), 1.92-1.85 (m, 1H), 1.81-1.69 (m, 1H), 1.66-1.57 (m, 1H), 1.51-1.42 (m, 1H), 1.30 (t, J=7.2 Hz, 3H).

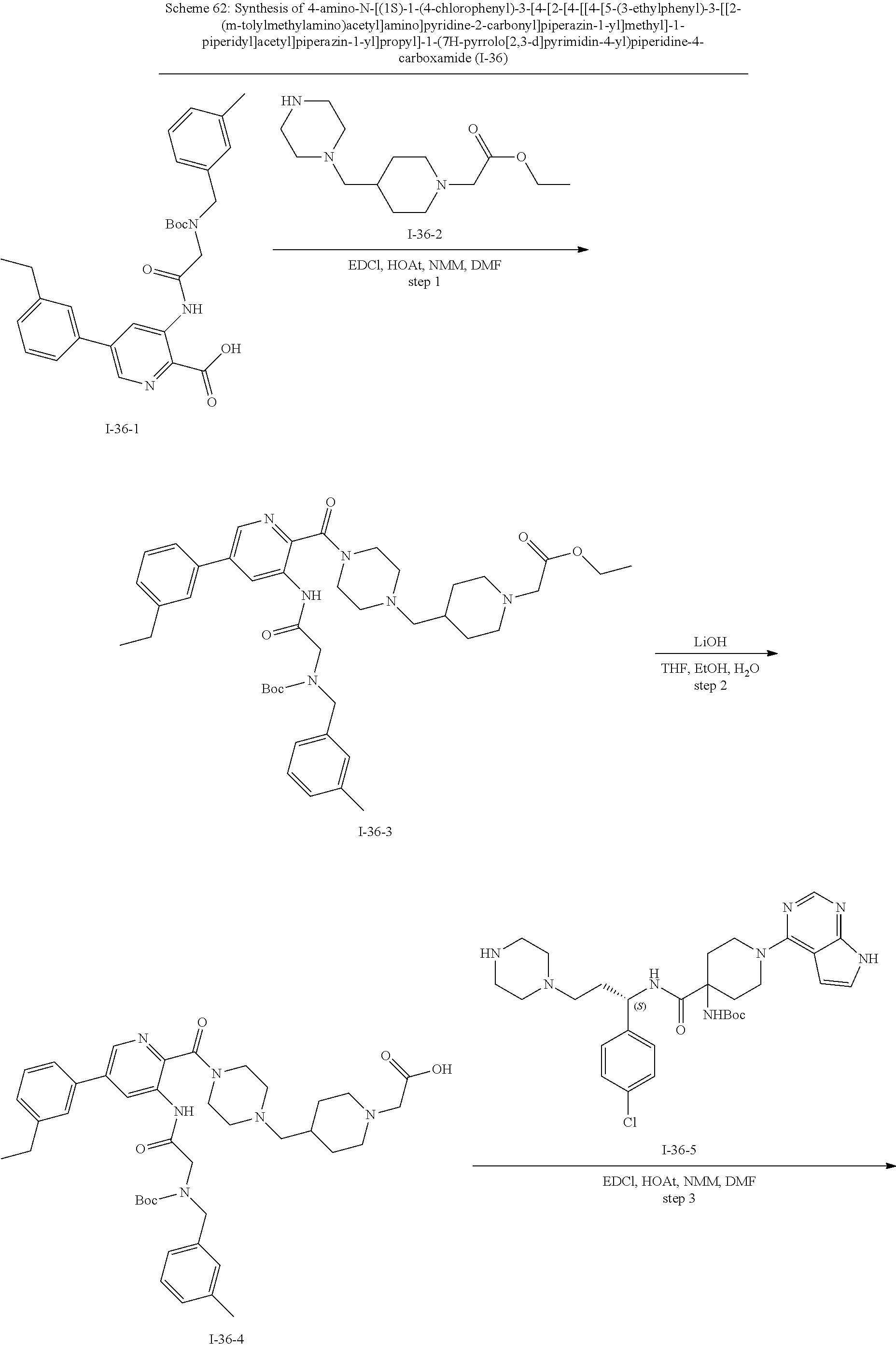

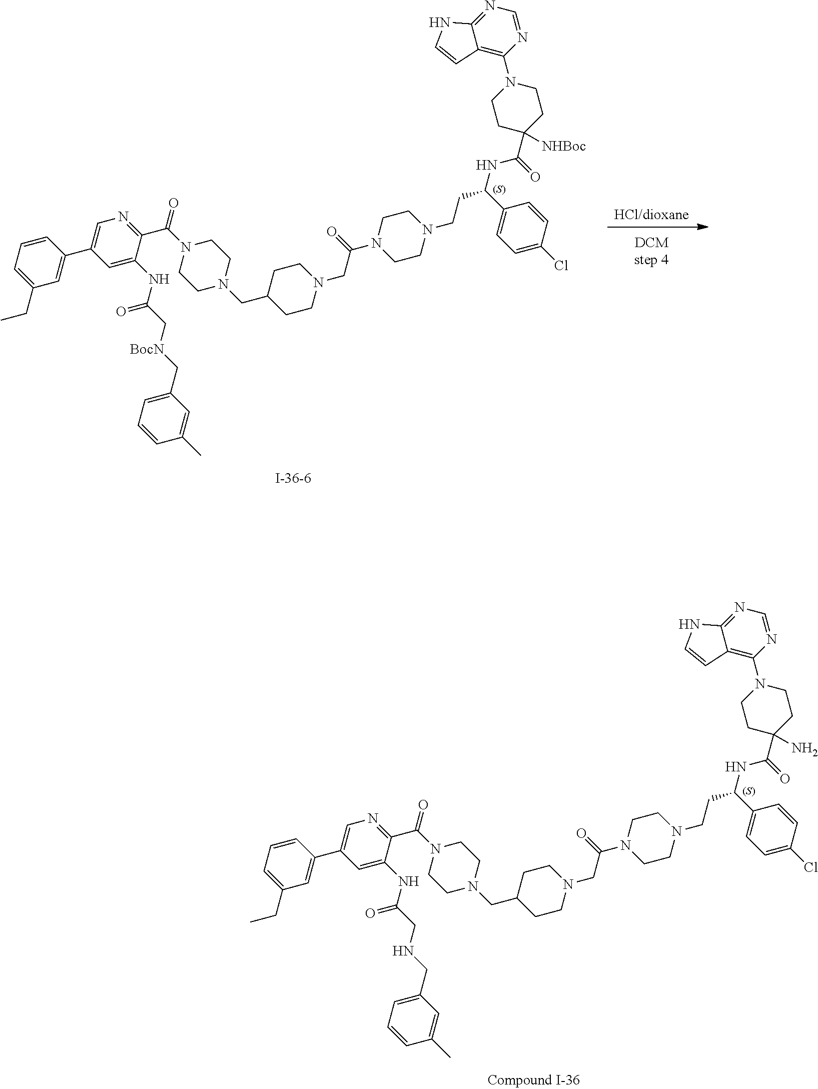

### Step 1: Synthesis of I-36-3

To a solution of I-36-1 (200 mg, 397.15 μmol, 1 eq) and I-36-2 (117.68 mg, 436.86 μmol, 1.1 eq) in DMF (2 mL) was added EDCI (228.40 mg, 1.19 mmol, 3 eq), HOAt (54.06 mg, 397.15 μmol, 1 eq) and NMM (401.71 mg, 3.97 mmol, 436.64 μL, 10 eq). The mixture was stirred at 25° C. for 1 hr. Upon completion, the mixture was poured into water (10 mL) and extracted with EA (10 mL×3). The combined organic layers were concentrated to give a residue. The residue was purified by flash silica gel chromatography (ISCO®; 20 g SepaFlash® Silica Flash Column, Eluent of 0˜37% MeOH/Ethyl acetate gradient @ 40 mL/min) and the eluent was concentrated to give I-36-3 (240 mg, 273.39 μmol, 68.84% yield, 86% purity) as a yellow solid. LCMS (Method E): R^{t}=0.501 min, [M+H]⁺=755.5.

### Step 2: Synthesis of I-36-4

To a solution of I-36-3 (240 mg, 317.90 μmol, 1 eq) in THF (2 mL), H₂O (1 mL) and EtOH (1 mL) was added LiOH (15.23 mg, 635.80 μmol, 2 eq). The mixture was stirred at 25° C. for 1 hr. Upon completion, the dry ice was added into the mixture to adjust PH˜8 then the mixture was concentrated to give I-36-4 (230 mg, crude, Lithium salt) as brown solid, which was used for next step directly.

### Step 3: Synthesis of I-36-6

To a solution of I-36-4 (230 mg, 313.42 μmol, 1 eq, Lithium salt) and I-36-5 (224.59 mg, 376.10 μmol, 1.2 eq) in DMF (2.5 mL) was added EDCI (180.25 mg, 940.26 μmol, 3 eq), HOAt (42.66 mg, 313.42 μmol, 1 eq) and NMM (317.01 mg, 3.13 mmol, 344.58 μL, 10 eq). The mixture was stirred at 25° C. for 1 hr. Upon completion, the mixture was poured into aq. NH₄Cl (15 mL) and extracted with EA (20 mL×3). The combined organic layers were concentrated to give a residue. The residue was purified by flash silica gel chromatography (ISCO®; 40 g SepaFlash® Silica Flash Column, Eluent of 0˜37% MeOH/Ethyl acetate gradient (@; 60 mL/min) and the eluent was concentrated to give I-36-6 (250 mg, 172.28 μmol, 54.97% yield, 90% purity) as a yellow solid. LCMS (Method E): R^{t}=0.509 min, [M+H]⁺=1305.8.

### Step 4: Synthesis of I-36

To a solution of I-36-6 (200 mg, 153.14 μmol, 1 eq) in DCM (2 mL) was added HCl/dioxane (2 M, 2 mL, 26.12 eq). The mixture was stirred at 25° C. for 1 hr. Upon completion, the reaction mixture was concentrated under reduced pressure to give the crude product. The product was purified by prep-HPLC (column: YMC-Actus Triart C18 150×30 mm×7 um; mobile phase: [water (FA)-ACN]; gradient: 5%-35% B over 10 min) and the eluent was concentrated to remove MeCN and then lyophilized to give I-36 (96.02 mg, 82.75 μmol, 54.03% yield, 99.26% purity, FA) was obtained as a white solid. LCMS (Method E): R^{t}=0.392 min, [M+H]⁺=1106.0. SFC: R^{t}=1.578 min. ¹H NMR (400 MHZ, METHANOL-d4) δ=8.60 (d, J=17.6 Hz, 2H), 8.44-8.39 (m, 2H), 8.15 (s, 1H), 7.54-7.47 (m, 2H), 7.46-7.39 (m, 1H), 7.36-7.28 (m, 6H), 7.26 (d, J=4.4 Hz, 2H), 7.18-7.09 (m, 2H), 6.64 (d, J=3.2 Hz, 1H), 5.03-4.96 (m, 1H), 4.62-4.49 (m, 2H), 4.02-3.89 (m, 4H), 3.79 (s, 2H), 3.70-3.57 (m, 6H), 3.53 (s, 2H), 3.44 (d, J=5.4 Hz, 4H), 2.87-2.80 (m, 2H), 2.74-2.65 (m, 2H), 2.57-2.40 (m, 8H), 2.40-2.33 (m, 5H), 2.33-2.19 (m, 4H), 2.09-1.94 (m, 4H), 1.82 (d, J=6.8 Hz, 1H), 1.73 (d, J=13.6 Hz, 2H), 1.56-1.42 (m, 2H), 1.29-1.20 (m, 3H),

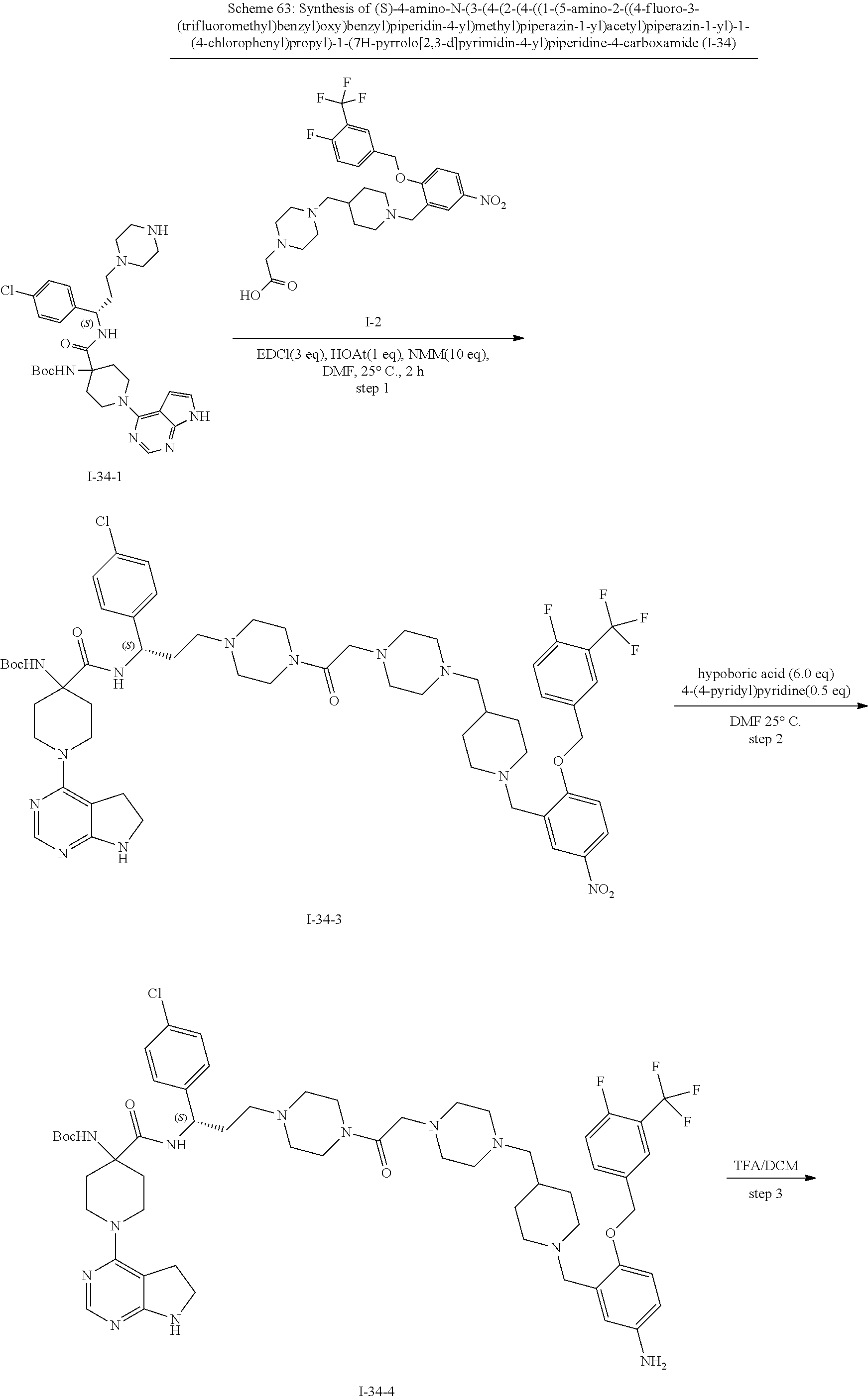

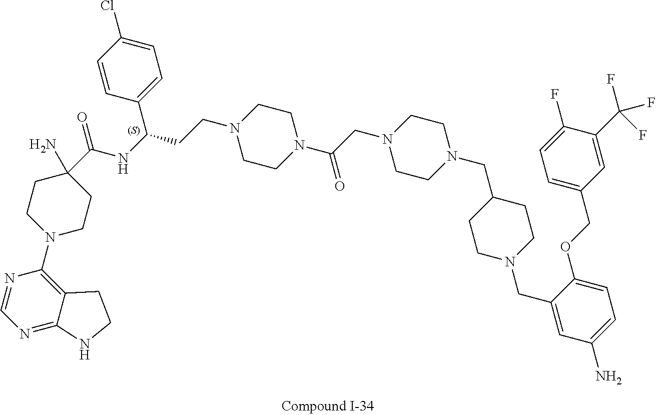

### Step 1: Synthesis of I-34-3

To a solution of I-34-1 (91.90 mg, 123.12 μmol, 1 eq) and I-34-2 (70 mg, 123.12 μmol, 1 eq) in DCM (1 mL) was added EDCI (70.81 mg, 369.35 μmol, 3 eq), HOAt (16.76 mg, 123.12 μmol, 1 eq) and NMM (124.53 mg, 1.23 mmol, 135.36 μL, 10 eq). The mixture was stirred at 25° C. for 2 h. Upon completion, the reaction mixture was quenched by addition H₂O 1 mL, and then diluted with DCM 1 mL and extracted with DCM 6 mL. The combined organic layers were dried over anhydrous Na₂SO₄, filtered, and concentrated under reduced pressure to give a residue. The residue was purified by flash silica gel chromatography (ISCO®; 4 g SepaFlash® Silica Flash Column, Eluent of 0˜100% Ethyl acetate: Methanol (@ 18 mL/min), concentrated under reduced pressure to give I-34-3 (90 mg, 68.22 μmol, 55.41% yield, 87% purity) as a white solid. LCMS: R^{t}=0.819 min, [M+H]⁺=1147.8.

### Step 2: Synthesis of I-34-4

To a solution I-34-3 (90 mg, 78.42 μmol, 1 eq) in DMF (1 mL) was added hypoboric acid (42.18 mg, 470.51 μmol, 6 eq) and 4-(4-pyridyl)pyridine (6.12 mg, 39.21 μmol, 0.5 eq). The mixture was stirred at 25° C. for 2 h. Upon completion, the reaction mixture was quenched by addition H₂O 1 mL, and then diluted with EA 1 mL and extracted with EA 6 mL. The combined organic layers were washed with brine 6 mL, dried over anhydrous Na₂SO₄, filtered, and concentrated under reduced pressure to give I-34-4 (70 mg, 62.63 μmol, 79.86% yield, 100% purity) as a white solid. LCMS (Method E): R^{t}=0.413 min, [M+H]⁺=1117.7.

### Step 3: Synthesis of I-34

To a solution of I-34-4 (60 mg, 53.68 μmol, 1 eq) in DCM (1 mL) was added TFA (767.50 mg, 6.73 mmol, 0.5 mL, 125.39 eq). The mixture was stirred at 25° C. for 1 h. Upon completion, the reaction mixture was concentrated under reduced pressure to give a residue. The residue was purified by prep-HPLC (column: Phenomenex luna C18 150×25 mm×10 um; mobile phase: [water (TFA)-ACN]; gradient: 5%-35% B over min), then concentrated to remove organic solvents and lyophilized to give I-34 (32.63 mg, 28.63 μmol, 53.33% yield, 99.28% purity; TFA) as a white solid. LCMS (Method E): R^{t}=0.392 min, [M+H]⁺=1017.6. SFC: R^{t}=1.667 min. ¹H NMR (400 MHZ, METHANOL-d4) δ=8.39 (s, 1H), 7.90-7.81 (m, 2H), 7.54 (d, J=2.4 Hz, 1H), 7.50-7.48 (m, 1H), 7.44-7.32 (m, 7H), 6.94 (d, J=4.0 Hz, 1H), 5.29 (s, 2H), 5.05-5.01 (m, 1H), 4.64-4.60 (m, 2H), 4.38 (s, 2H), 3.95-3.85 (m, 4H), 3.84-3.69 (m, 2H), 3.64-3.47 (m, 3H), 3.46-3.32 (m, 3H), 3.28 (d, J=4.8 Hz, 1H), 3.24-2.80 (m, 13H), 2.80-2.72 (m, 2H), 2.71-2.56 (m, 2H), 2.43-2.25 (m, 2H), 2.19 (d, J=14.4 Hz, 1H), 2.10-1.96 (m, 4H), 1.53 (d, J=12.4 Hz, 2H). ¹⁹FNMR (377 MHz, METHANOL-d4) δ=−62.755, −76.992, −117.310.

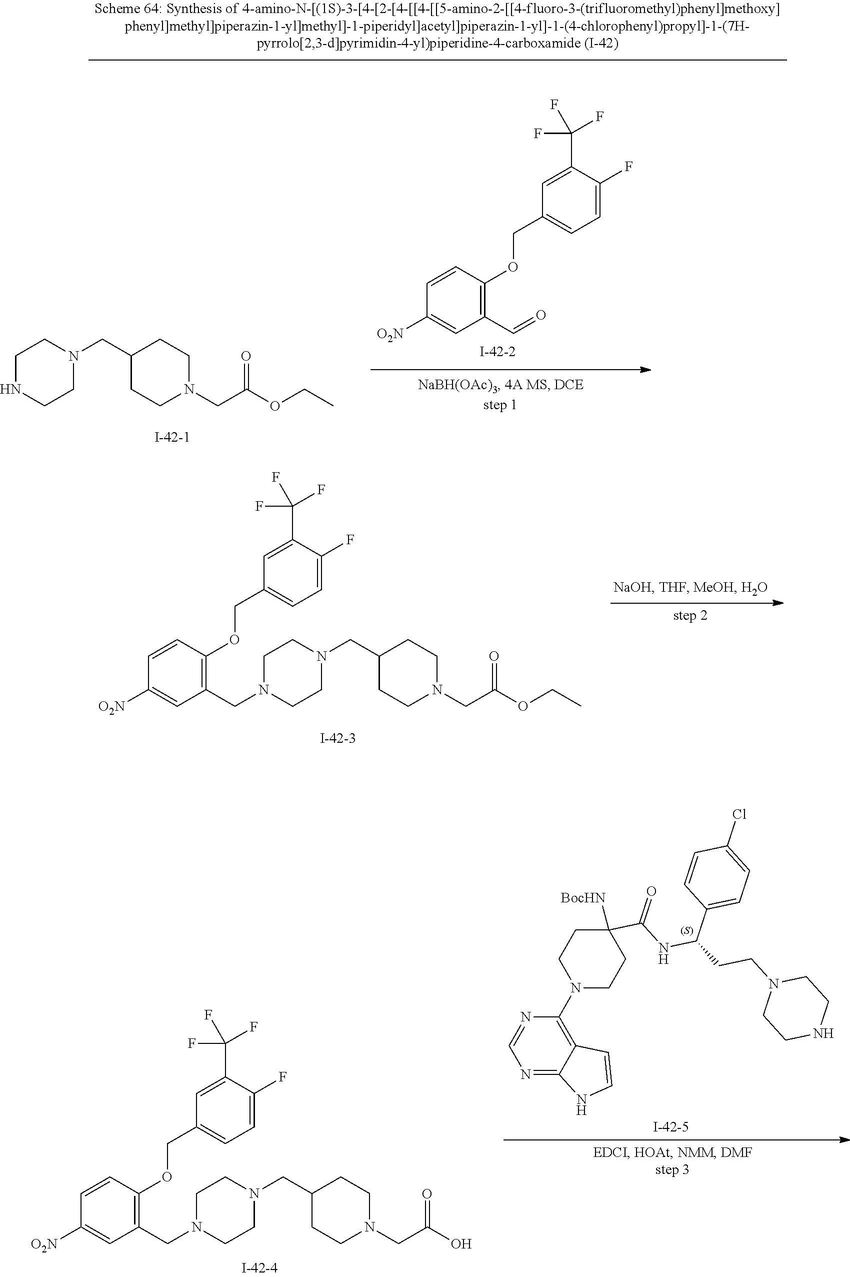

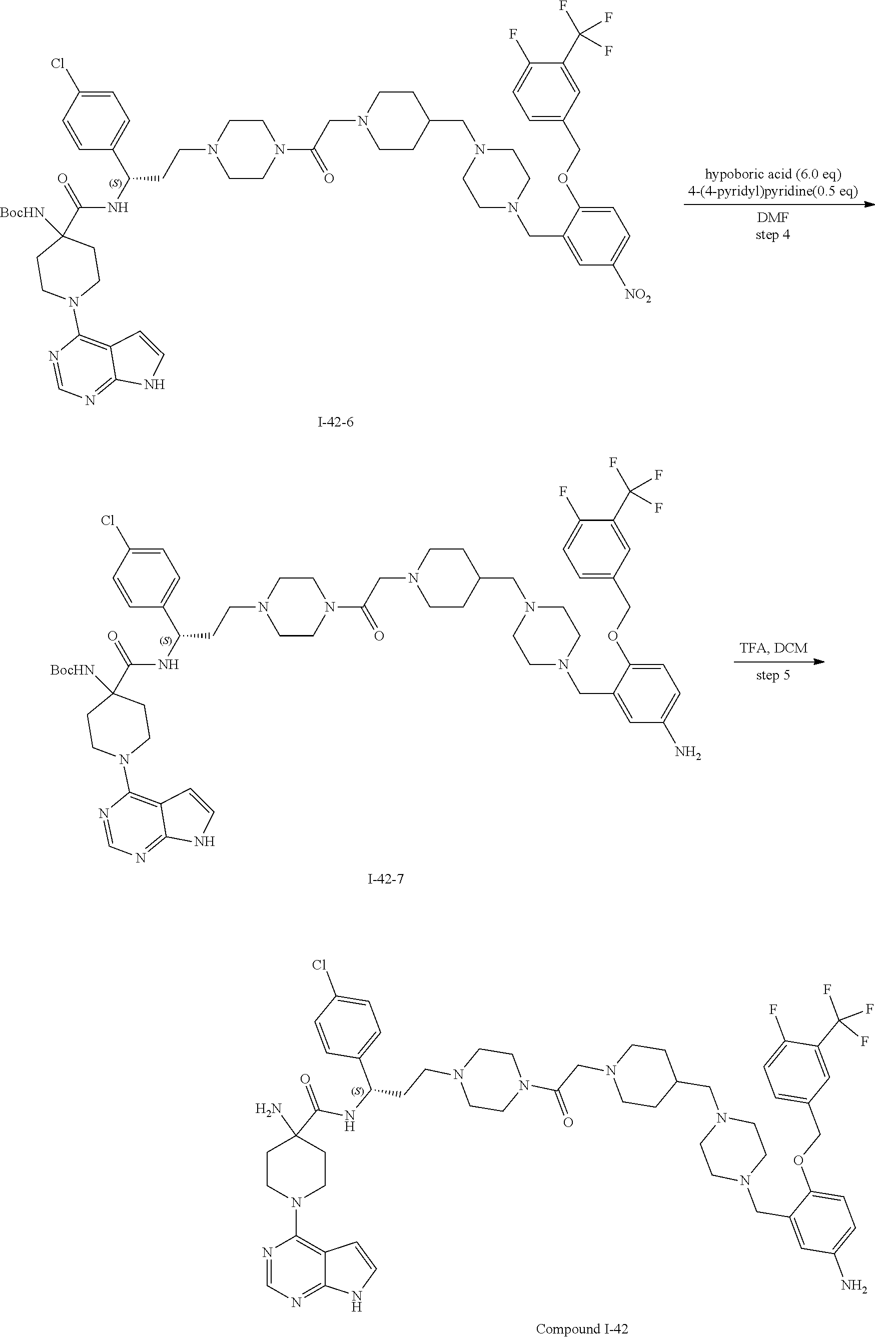

### Step 1: Synthesis of I-42-3

A solution of I-42-1 (600 mg, 1.96 mmol, 1 eq, HCl) and 4 A MS (200 mg) in DCE (6 mL) was stirred at 25° C. for 0.25 h under N₂ atmosphere. Then I-42-2 (808.01 mg, 2.35 mmol, 1.2 eq) was added to the mixture, and the mixture was stirred at 25° C. for 0.75 h, then NaBH(OAc)₃ (1.04 g, 4.90 mmol, 2.5 eq) was added to the mixture and the mixture was stirred at 25° C. for 1 hr. Upon completion, the mixture was added NaHCO₃ to adjust PH≥8, then the mixture was poured into water 10 mL and extracted with EA (20 mL×3). The combined organic layers were concentrated to give a residue. The residue purified by flash silica gel chromatography (ISCO®; 20 g SepaFlash® Silica Flash Column, Eluent of 0˜40% MeOH/Ethyl acetate gradient (@) 40 mL/min) and the eluent was concentrated to give I-42-3 (290 mg, 465.61 μmol, 23.73% yield, 95.79% purity) as a yellow solid. LCMS (Method E): R^{t}=0.436 min, [M+H]⁺=597.3.

### Step 2: Synthesis of I-42-4

To a solution of I-42-3 (150 mg, 251.42 μmol, 1 eq) in THF (1.5 mL) and MeOH (1.5 mL) was added NaOH (20.11 mg, 502.84 μmol, 2 eq) in H₂O (0.5 mL). The mixture was stirred at 25° C. for 1 hr. Upon completion, the reaction mixture was concentrated under reduced pressure to give the crude product. The mixture was purified by reversed phased HPLC (FA), the target peak was concentrated to remove MeCN and then extracted with EA (10 mL×2). The organic layers were dried over anhydrous sodium sulfate, filtered, and concentrated to give I-42-4 (110 mg, 192.72 μmol, 76.65% yield, 99.61% purity) as a white solid. LCMS (Method E): R^{t}=0.435 min, [M+H]⁺=569.2.

### Step 3: Synthesis of I-42-6

To a solution of I-42-4 (60 mg, 105.53 μmol, 1 eq) and I-42-5 (102.40 mg, 137.19 μmol, 1.3 eq) in DMF (0.6 mL) was added EDCI (60.69 mg, 316.59 μmol, 3 eq), HOAt (14.36 mg, 105.53 μmol, 1 eq) and NMM (106.74 mg, 1.06 mmol, 116.02 μL, 10 eq). The mixture was stirred at 25° C. for 2 hr. Upon completion, the mixture was poured into water 10 mL and extracted with EA (10 mL×3). The combined organic layers were concentrated to give a residue. The residue was purified by reversed phased HPLC (FA), the target peak was concentrated and then extracted with EA (10 mL×2), and the organic layers were dried over anhydrous sodium sulfate, filtered, and concentrated to give I-42-6 (50 mg, 40.95 μmol, 38.81% yield, 94% purity) as yellow oil. LCMS (Method E): R^{t}=0.524 min, [M+H]⁺=1148.6.

### Step 4: Synthesis of I-42-7

To a solution of I-42-6 (42 mg, 36.60 μmol, 1 eq) in DMF (0.4 mL) was added 4-(4-pyridyl)pyridine (2.86 mg, 18.30 μmol, 0.5 eq). Then hypoboric acid (19.68 mg, 219.57 μmol, 6 eq) was added at 0° C. The mixture was stirred at 25° C. for 1 hr. Upon completion, the mixture was poured into water 10 mL and extracted with EA (5 mL×3). The combined organic layers were concentrated to give I-42-7 (35 mg, 29.12 μmol, 79.58% yield, 93% purity) as yellow oil. LCMS (Method E): R^{t}=0.401 min, [M+H]⁺=1117.8.

### Step 5: Synthesis of I-42

To a solution of I-42-7 (35 mg, 31.31 μmol, 1 eq) in DCM (2 mL) was added TFA (1.07 g, 9.42 mmol, 700 μL, 300.94 eq). The mixture was stirred at 25° C. for 1 hr. Upon completion, the reaction mixture was concentrated under reduced pressure to give the crude product. The product was purified by prep-HPLC (column: Phenomenex luna C18 150×25 mm×10 um; mobile phase: [water (TFA)-ACN]; gradient: 8%-38% B over min) and the eluent was concentrated to remove MeCN and then lyophilization. I-42 (17.57 mg, 15.53 μmol, 49.58% yield, 100% purity, TFA) was obtained as a yellow solid. LCMS (Method E): R^{t}=0.400 min, [M+H]⁺=1017.8. SFC: R^{t}=1.792 min, ee value=100%. ¹H NMR (400 MHZ, METHANOL-d4) δ=8.41-8.32 (m, 1H), 7.87-7.80 (m, 2H), 7.44-7.41 (m, 1H), 7.40-7.33 (m, 7H), 7.28-7.25 (m, 1H), 6.91 (d, J=3.6 Hz, 1H), 5.25 (s, 2H), 5.03-5.00 (m, 1H), 4.68-4.57 (m, 2H), 4.33-4.23 (m, 2H), 4.14 (s, 2H), 3.91-3.78 (m, 3H), 3.76-3.59 (m, 4H), 3.48-3.34 (m, 3H), 3.26 (d, J=4.8 Hz, 1H), 3.22-2.79 (m, 12H), 2.77-2.50 (m, 5H), 2.41-2.23 (m, 2H), 2.17 (d, J=14.4 Hz, 1H), 2.08-1.98 (m, 4H), 1.68-1.51 (m, 2H). ¹⁹F NMR (377 MHz, METHANOL-d4) δ=−62.777, −77.003, −117.609.

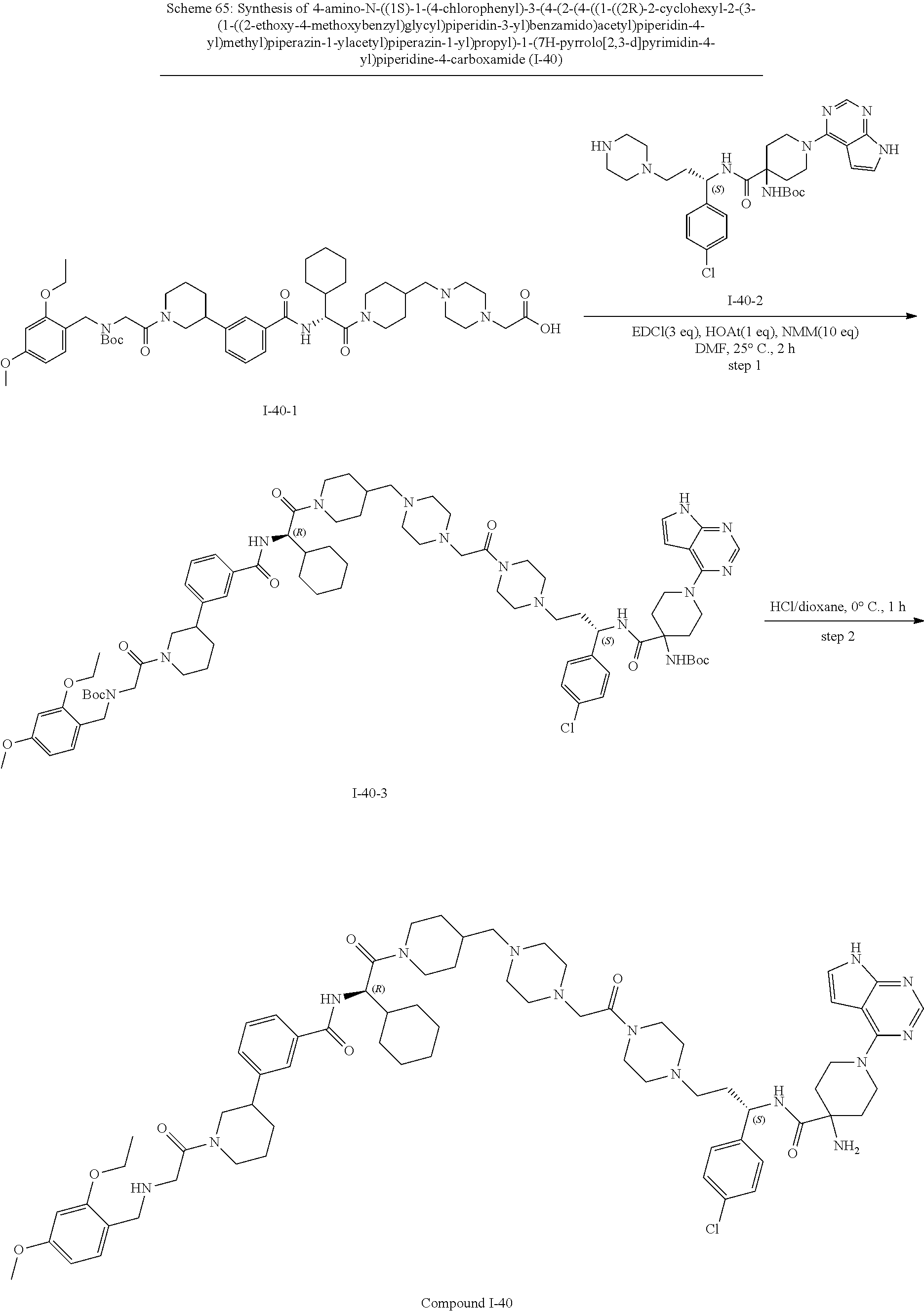

### Step 1: Synthesis of I-40-3

To a solution of I-40-1 (60 mg, 67.48 μmol, 1 eq) and I-40-2 (60.45 mg, 80.98 μmol, 1.2 eq) in DCM (1 mL) was added EDCI (38.81 mg, 202.45 μmol, 3 eq), HOAt (9.19 mg, 67.48 μmol, 1 eq) and NMM (68.26 mg, 674.82 μmol, 74.19 μL, 10 eq). The mixture was stirred at 25° C. for 2 h. Upon completion, the reaction mixture was quenched by addition H₂O 1 mL, and then diluted with DCM 1 mL and extracted with DCM 6 mL. The combined organic layers were dried over anhydrous Na₂SO₄, filtered, and concentrated under reduced pressure to give a residue. The residue was purified by prep-HPLC (column: Waters Xbridge 150×25 mm×5 um; mobile phase: [water (ammonia hydroxide v/v)-ACN]; gradient: 58%-88% B over min), then concentrated to remove organic solvents and lyophilized to give I-40-3 (20 mg, 13.62 μmol, 20.19% yield, 100% purity) as a white solid. LCMS (Method E): R^{t}=0.554 min, [M+H]⁺=1469.1.

### Step 2: Synthesis of I-40

To a solution of I-40-3 (15 mg, 10.22 μmol, 1 eq) in HCl/dioxane (4M, 0.75 mL). The mixture was stirred at 0° C. for 1 h. Upon completion, the reaction mixture was concentrated under reduced pressure to give a residue, and purified by prep-HPLC (column: Waters Xbridge 150×25 mm×5 um; mobile phase: [water (ammonia hydroxide v/v)-ACN]; gradient: 43%-73% B over min), then concentrated to remove organic solvents and lyophilized to give I-40 (9.21 mg, 7.25 μmol, 70.92% yield, 99.75% purity) as a white solid. LCMS (Method G): R^{t}=0.790 min, [M+H]⁺=1267.9. SFC: R^{t}=3.153 min, R^{t}=4.145 min. ¹H NMR (400 MHZ, METHANOL-d4) δ=8.12 (s, 1H), 7.77-7.66 (m, 2H), 7.49-7.38 (m, 2H), 7.37-7.29 (m, 4H), 7.17-7.09 (m, 2H), 6.62-6.21 (m, 1H), 6.55-6.41 (m, 2H), 4.97 (d, J=7.6 Hz, 2H), 4.57-4.45 (m, 4H), 4.33-4.26 (m, 1H), 4.09-4.01 (m, 2H), 3.83-3.71 (m, 6H), 3.69-3.58 (m, 6H), 3.46 (d, J=6.4 Hz, 1H), 3.22-3.09 (m, 4H), 2.80-2.61 (m, 4H), 2.55-2.41 (m, 9H), 2.39-2.32 (m, 3H), 2.26-2.13 (m, 4H), 2.09-1.94 (m, 4H), 1.93-1.72 (m, 10H), 1.71-1.50 (m, 5H), 1.46-1.38 (m, 3H), 1.33-1.04 (m, 7H).

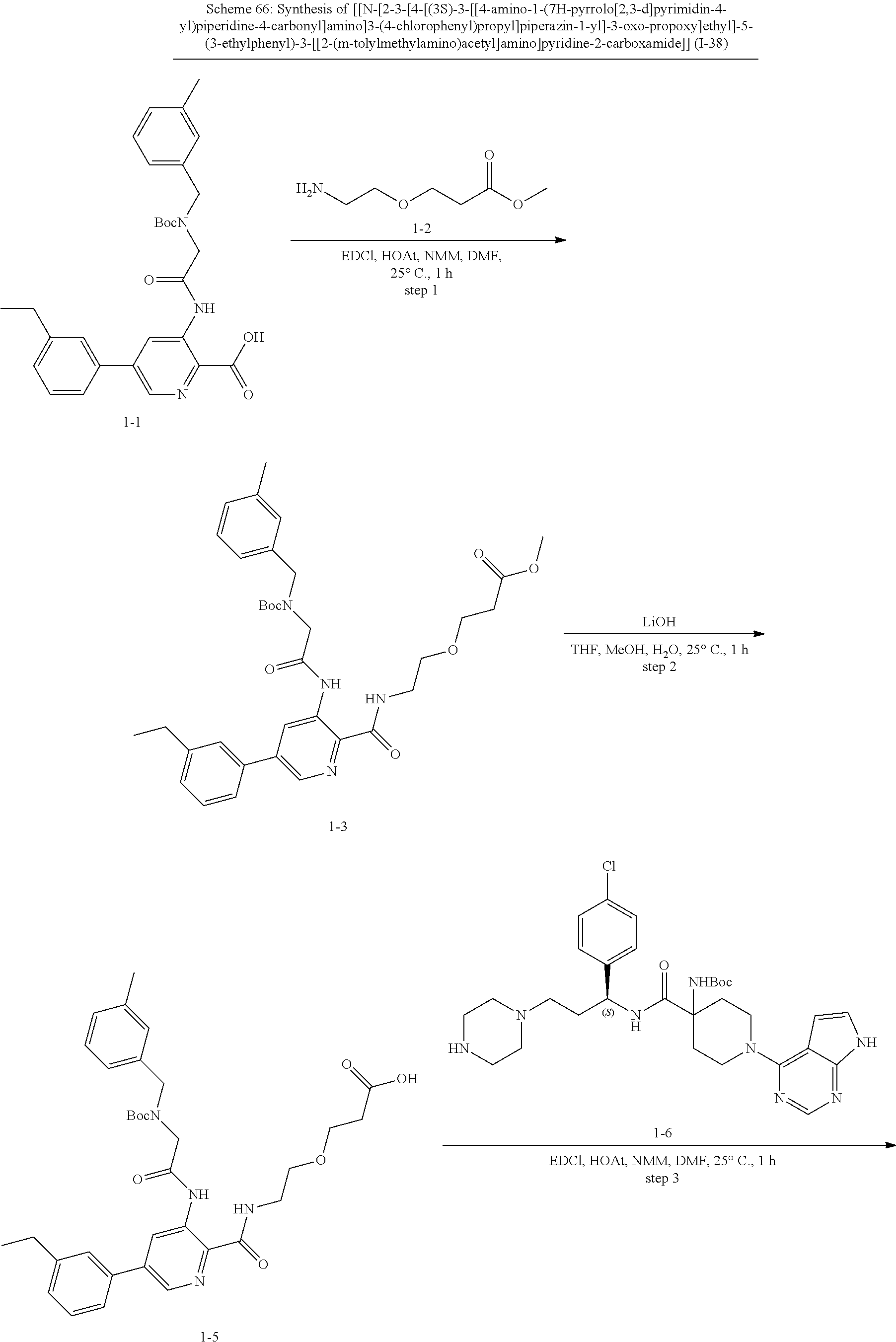

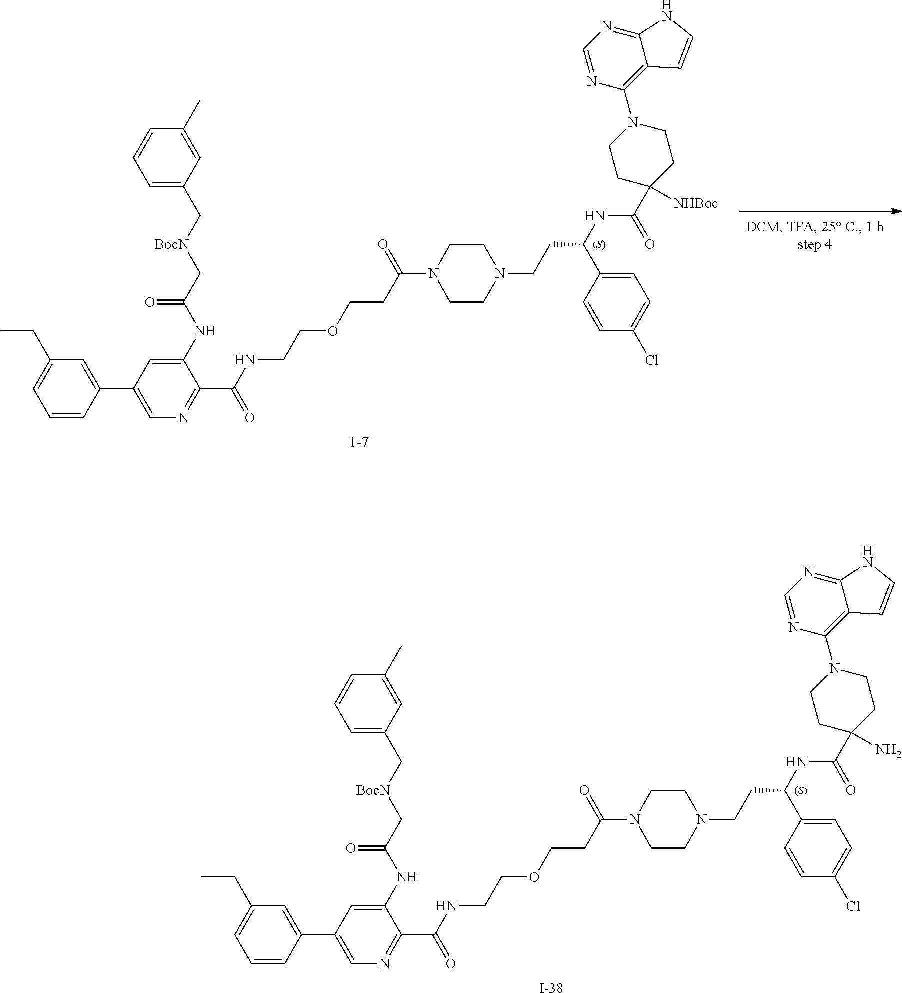

### Step 1: Synthesis of I-38-3

To a solution of I-38-1 (200 mg, 397.15 μmol, 1 eq) in DMF (1 mL) was added HOAt (108.11 mg, 794.30 μmol, 111.11 μL, 2 eq), EDCI (380.67 mg, 1.99 mmol, 5 eq) and NMM (401.71 mg, 3.97 mmol, 436.64 μL, 10 eq), then the I-38-2 (116.90 mg, 794.30 μmol, 2 eq) was added. The mixture was stirred at 25° C. for 4 hr. The reaction mixture was poured into water (10 mL) and extracted with EA 20 mL (5 mL×4). The combined organic layers were dried over Na₂SO₄, filtered, and concentrated under reduced pressure to give a residue. The residue was purified by flash silica gel chromatography (ISCO®; 12 g SepaFlash® Silica Flash Column, Eluent of 0˜100% Ethyl acetate/Petroleum ether gradient @ 50 mL/min), the eluent was concentrated under reduced pressure to give a residue. I-38-3 (190 mg, 300.28 μmol, 75.61% yield) was obtained as a white solid. LCMS (Method D): R^{t}=0.596 min, [M+H]⁺=633.3.

### Step 2: Synthesis of I-38-4

To a solution of I-38-3 (180 mg, 284.47 μmol, 1 eq) in THF (0.6 mL), MeOH (0.6 mL) and H₂O (0.6 mL) was added LiOH·H₂O (23.88 mg, 568.95 μmol, 2 eq). The mixture was stirred at 25° C. for 0.5 h. Upon completion, the reaction mixture was adjusted to pH=6 with CA, and then extracted with EA (10 mL×3). The combined organic layers dried over Na₂SO₄, filtered, and concentrated under reduced pressure to give a residue. The residue was used into the next step without further purification. The crude product I-38-4 (160 mg, 258.60 μmol, 90.90% yield) was obtained as a white oil. LCMS (Method D): R^{t}=0.572 min, [M+H]⁺=619.6.

### Step 3: Synthesis of I-38-6

To a solution of I-38-4 (45.59 mg, 73.68 μmol, 1.1 eq) in DMF (0.5 mL) was added HOAt (18.23 mg, 133.97 μmol, 18.74 μL, 2 eq), EDCI (64.21 mg, 334.92 μmol, 5 eq) and NMM (67.75 mg, 669.85 μmol, 73.64 μL, 10 eq), then the I-38-5 (40 mg, 66.98 μmol, 1 eq) was added in. The mixture was stirred at 25° C. for 1 hr. Upon completion, the reaction mixture was poured into water (5 mL) and extracted with EA 20 mL (5 mL×4). The combined organic layers were dried over Na₂SO₄, filtered, and concentrated under reduced pressure to give a residue. The residue was used to the next step without further purification. I-38-6 (75 mg, 62.61 μmol, 93.47% yield) was obtained as a yellow oil. LCMS (Method D): R^{t}=0.497 min, [M+H]⁺=1198.6.

### Step 4: Synthesis of I-38

To a solution of I-38-6 (70 mg, 58.44 μmol, 1 eq) in DCM (0.4 mL) was added TFA (307.00 mg, 0.2 mL). The mixture was stirred at 25° C. for 1 hr. Upon completion, the reaction mixture was concentrated under reduced pressure to give a residue. The residue was purified by prep-HPLC (column: Phenomenex luna C18 150×25 mm×10 um; mobile phase: [water (TFA)-ACN]; gradient: 20%-50% B over 15 min). The eluent was lyophilization to give product. I-38 (30 mg, 29.69 μmol, 50.81% yield, 98.73% purity) was obtained as a pink solid. LCMS (Method D): R^{t}=0.363 min, [M+H]⁺=997.3. ¹H NMR (400 MHZ, METHANOL-d4) δ=9.21 (s, 1H), 8.62 (s, 1H), 8.33 (s, 1H), 7.50 (d, J=12.0 Hz, 2H), 7.44 (t, J=7.6 Hz, 1H), 7.40-7.35 (m, 3H), 7.34-7.27 (m, 8H), 6.85 (d, J=2.8 Hz, 1H), 5.03-4.96 (m, 2H), 4.64-4.58 (m, 2H), 4.29 (s, 2H), 4.17 (s, 2H), 3.80 (t, J=5.6 Hz, 5H), 3.66 (d, J=5.2 Hz, 2H), 3.61 (d, J=4.8 Hz, 2H), 3.28-3.18 (m, 4H), 3.15-3.01 (m, 2H), 2.79-2.68 (m, 5H), 2.66-2.49 (m, 3H), 2.39 (s, 3H), 2.36-2.31 (m, 1H), 2.29-2.22 (m, 1H), 2.13 (d, J=13.6 Hz, 1H), 2.00 (d, J=14.4 Hz, 1H), 1.29 (t, J=7.6 Hz, 3H).

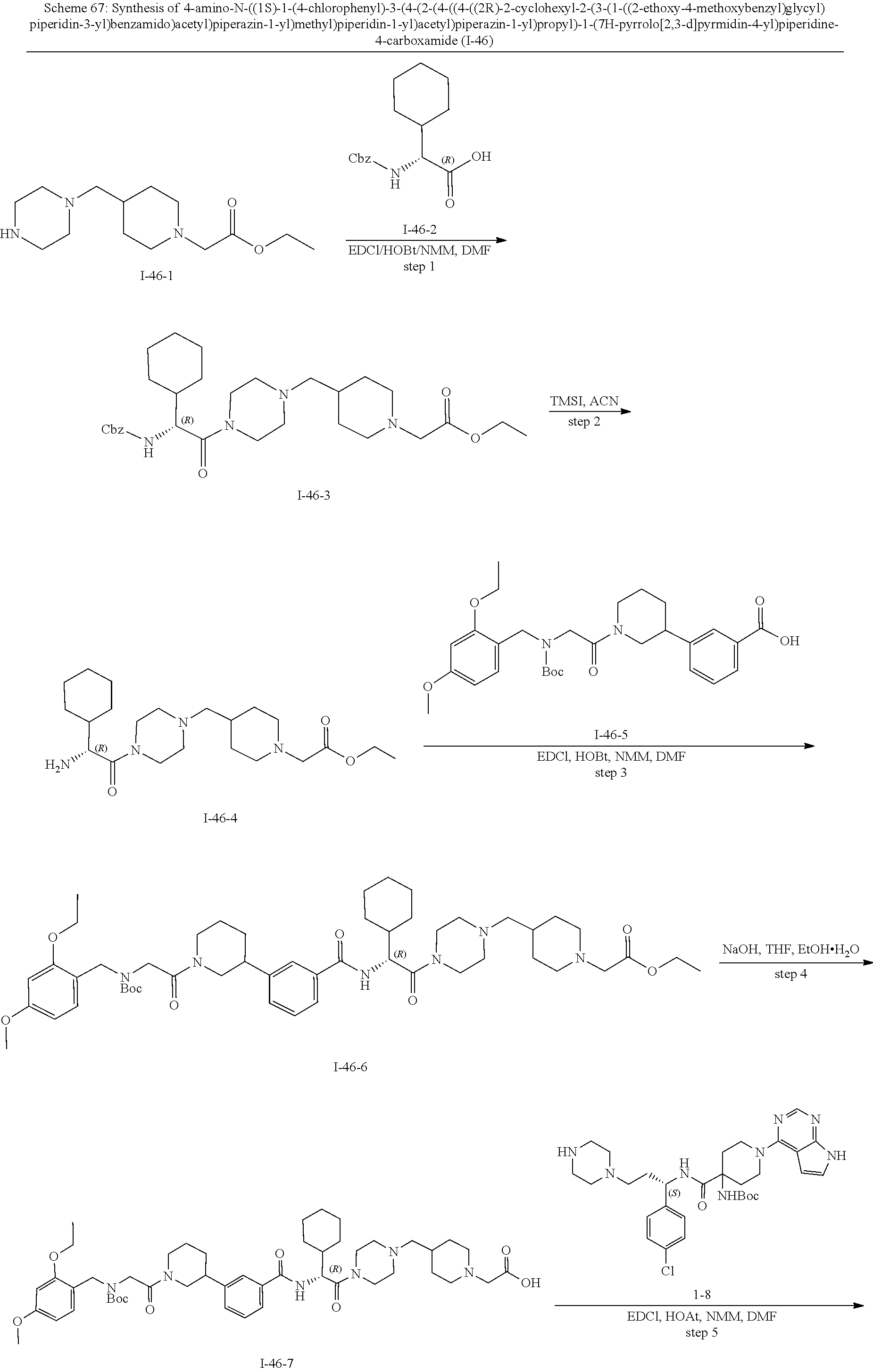

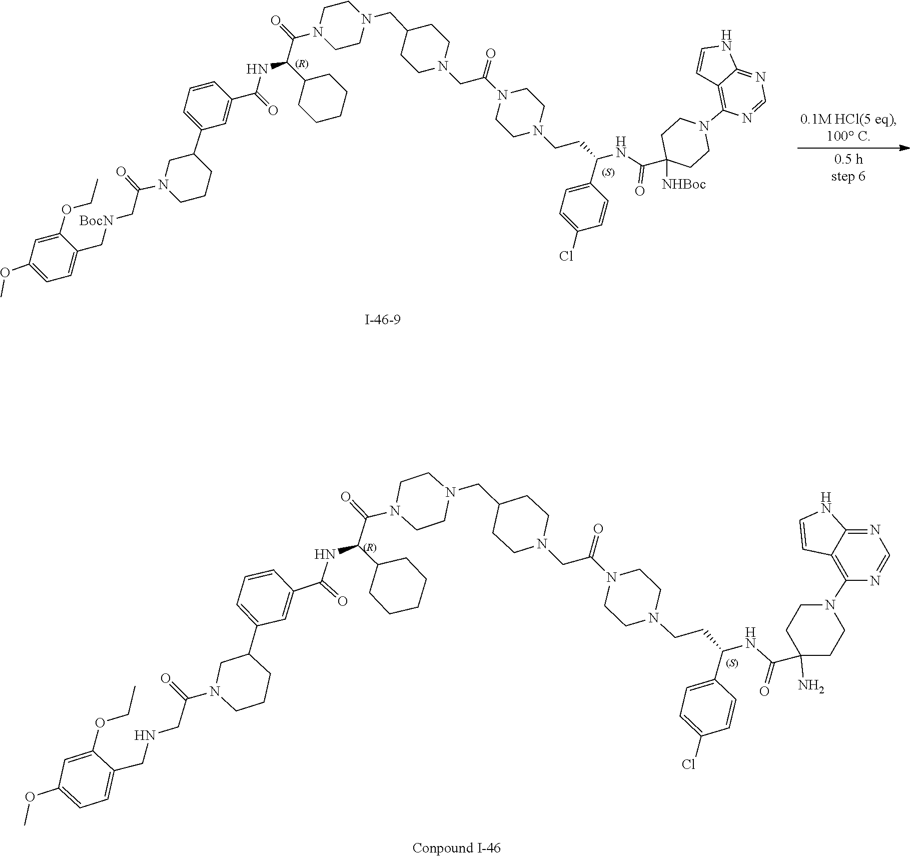

### Step 1: Synthesis of I-46-3

To a solution of I-46-2 (2.29 g, 7.85 mmol, 1.2 eq), HOBt (883.61 mg, 6.54 mmol, 1 eq), EDCI (3.76 g, 19.62 mmol, 3 eq) and NMM (6.61 g, 65.39 mmol, 7.19 mL, 10 eq) in DMF (20 mL) was added I-46-1 (2 g, 6.54 mmol, 1 eq, HCl). The mixture was stirred at 25° C. for 1 h. Upon completion, the reaction mixture was quenched by H₂O (40 mL), extracted with EA (20 mL×3). The combined organic layers were washed with brine (20 mL×3), dried over anhydrous Na₂SO₄, filtered, and concentrated under reduced pressure to give a residue. The residue was purified by column chromatography (SiO₂, DCM/MeOH=1/0 to 10/1) and concentrated to afford I-46-3 (3.3 g, 6.08 mmol, 92.99% yield) as yellow oil. LCMS: R^{t}=0.697 min, [M+H]⁺=543.4. SFC: R^{t}=1.612 min.

### Step 2: Synthesis of I-46-4

To a solution of I-46-3 (3.3 g, 6.08 mmol, 1 eq) in ACN (33 mL) was added TMSI (7.30 g, 36.48 mmol, 4.97 mL, 6 eq) at 0° C. Then the mixture was stirred at 0° C. for 2 h. Upon completion, the reaction mixture was quenched by addition H₂O 40 mL, and extracted with EA (20 mL×3). The aqueous was adjusted pH to 7-8 with NaHCO₃ saturated solution, then extracted with CHCl₃: i-PrOH (3:1) (30 mL×3). The organic layers were dried over anhydrous Na₂SO₄, filtered, and concentrated under reduced pressure to give I-46-4 (1.87 g, crude) a yellow oil, which was used for next directly. LCMS: R^{t}=0.550 min, [M+H]⁺=409.3.

### Step 3: Synthesis of I-46-6

To a solution of I-46-5 (2.65 g, 5.03 mmol, 1.1 eq), HOBt (618.44 mg, 4.58 mmol, 1 eq), EDCI (1.75 g, 9.15 mmol, 2 eq) and NMM (2.31 g, 22.88 mmol, 2.52 mL, 5 eq) in DMF (20 mL) was added I-46-4 (1.87 g, 4.58 mmol, 1 eq). The mixture was stirred at 25° C. for 1 h. Upon completion, the reaction mixture was quenched by H₂O (40 mL), extracted with EA (20 mL×3). The combined organic layers were washed with brine (20 mL×3), dried over anhydrous Na₂SO₄, filtered, and concentrated under reduced pressure to give a residue. The residue was purified by column chromatography (SiO₂, DCM/MeOH=1/0 to 10/1) and concentrated to give I-46-6 (3.4 g, 3.71 mmol, 80.99% yield) as yellow oil. LCMS: R^{t}=0.835 min, [M+H]⁺=917.7.

### Step 4: Synthesis of I-46-7

To a solution of I-46-6 (3.4 g, 3.71 mmol, 1 eq) in H₂O (7.4 mL), THF (34 mL) and EtOH (34 mL) was added NaOH (296.54 mg, 7.41 mmol, 2 eq). The mixture was stirred at 25° C. for 1 h. The mixture was concentrated under vacuum to remove THF and EtOH. The aqueous phase was adjusted to pH=7 by citric acid, then purified by reversed-phase HPLC (0.1% NH₃·H₂O) to give I-46-7 (2 g, 2.21 mmol, 59.67% yield, 98.336% purity) as a white solid. LCMS: R^{t}=0.610 min, [M+H]⁺=889.6. SFC: R^{t}=3.317 min, 3.899 min.

### Step 5: Synthesis of I-46-9

To a solution of I-46-7 (2 g, 2.25 mmol, 1 eq) in DMF (20 mL) was added HOAt (306.17 mg, 2.25 mmol, 1 eq), EDCI (1.29 g, 6.75 mmol, 3 eq), NMM (2.28 g, 22.49 mmol, 2.47 mL, 10 eq) and I-46-8 (1.48 g, 2.47 mmol, 1.1 eq). The mixture was stirred at 25° C. for 1 h. The mixture was poured into H₂O (100 ml), filtered, and the filter cake was washed with H₂O (10 ml×3) and concentrated under vacuum to give I-46-9 (3.7 g, crude) as a yellow solid. LCMS: R^{t}=0.854 min, [M+H]⁺=1469.1.

### Step 6: Synthesis of I-46

To a solution of I-46-9 (3.6 g, 2.45 mmol, 1 eq) in DCM (18 mL) was added HCl/dioxane (2 M, 36.00 mL, 29.37 eq) at 0° C., the mixture was stirred at 0° C. for 1 h. The reaction mixture was quenched by Sat. NaHCO₃ (100 mL), extracted with DCM (50 mL×3). The combined organic layers were washed with brine (50 mL×3), dried over anhydrous Na₂SO₄, filtered, and concentrated under reduced pressure to give a residue. The residue was purified by reversed-phase HPLC (0.1% NH₃·H₂O) and Prep-HPLC (column: Waters Xbridge 150×25 mm×5 um; mobile phase: [water (ammonia hydroxide v/v)-ACN]; gradient: 38%-68% B over 10 min) to give I-46 (948.66 mg, 746.48 μmol, 30.45% yield, 99.778% purity) as a white solid. LCMS: R^{t}=0.886 min, [M+H]⁺=1267.9. SFC: R^{t}=3.370 min, 5.011 min. ¹H NMR (400 MHZ, METHANOL-d4) δ=8.12 (s, 1H), 7.76-7.68 (m, 2H), 7.47-7.40 (m, 2H), 7.32 (s, 4H), 7.09-7.17 (m, 1H), 7.11 (d, J=3.6 Hz, 1H), 6.61 (d, J=3.6 Hz, 1H), 6.57-6.45 (m, 2H), 4.97-4.99 (m, 1H), 4.55-4.43 (m, 3H), 4.12-4.02 (m, 2H), 3.96-3.54 (m, 19H), 3.24 (s, 2H), 3.19-3.07 (m, 1H), 2.93 (br d, J=11.2 Hz, 2H), 2.83-2.64 (m, 2H), 2.56-2.30 (m, 11H), 2.30-1.94 (m, 10H), 1.94-1.71 (m, 9H), 1.70-1.52 (m, 6H), 1.47-1.39 (m, 3H), 1.32-1.08 (m, 7H).

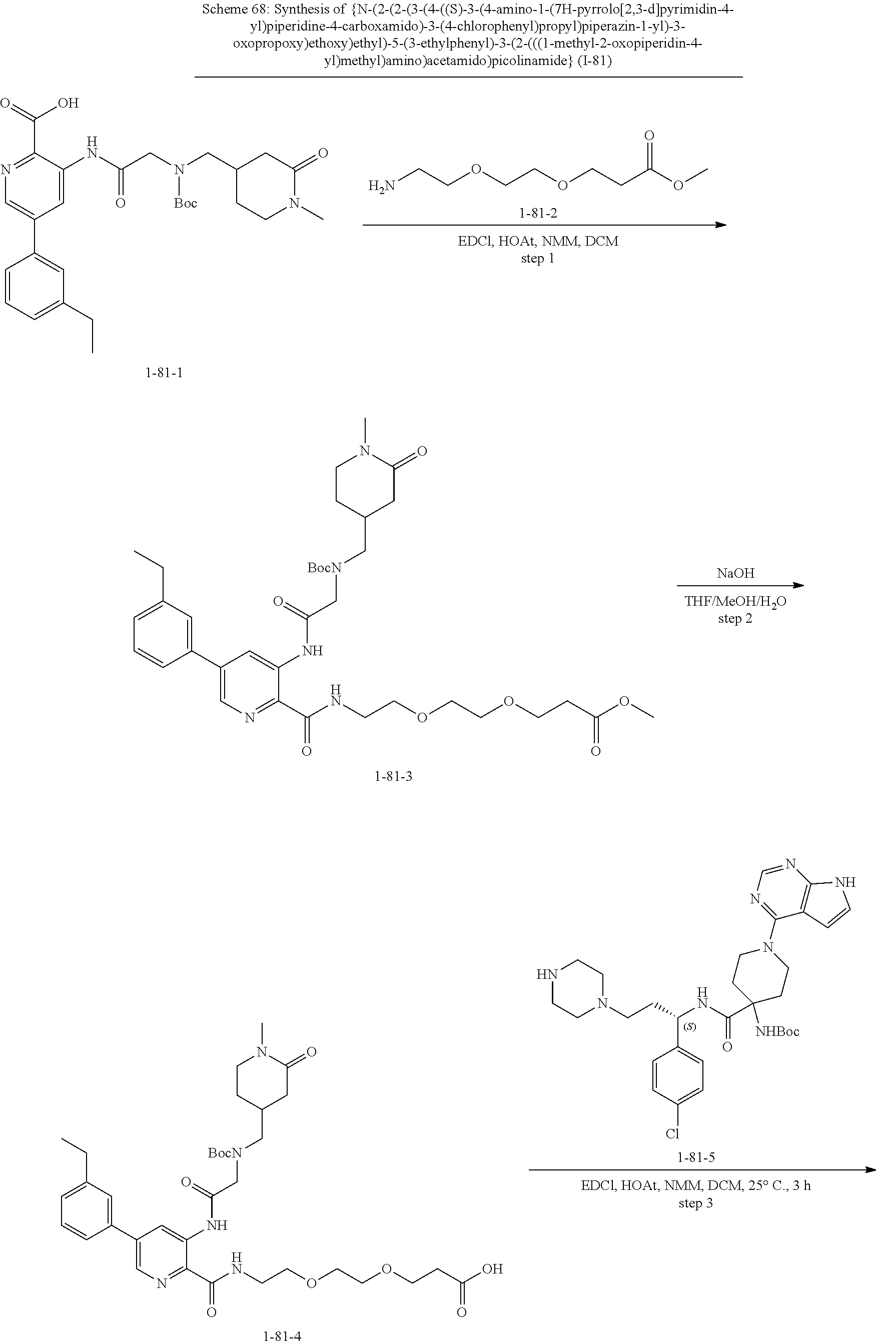

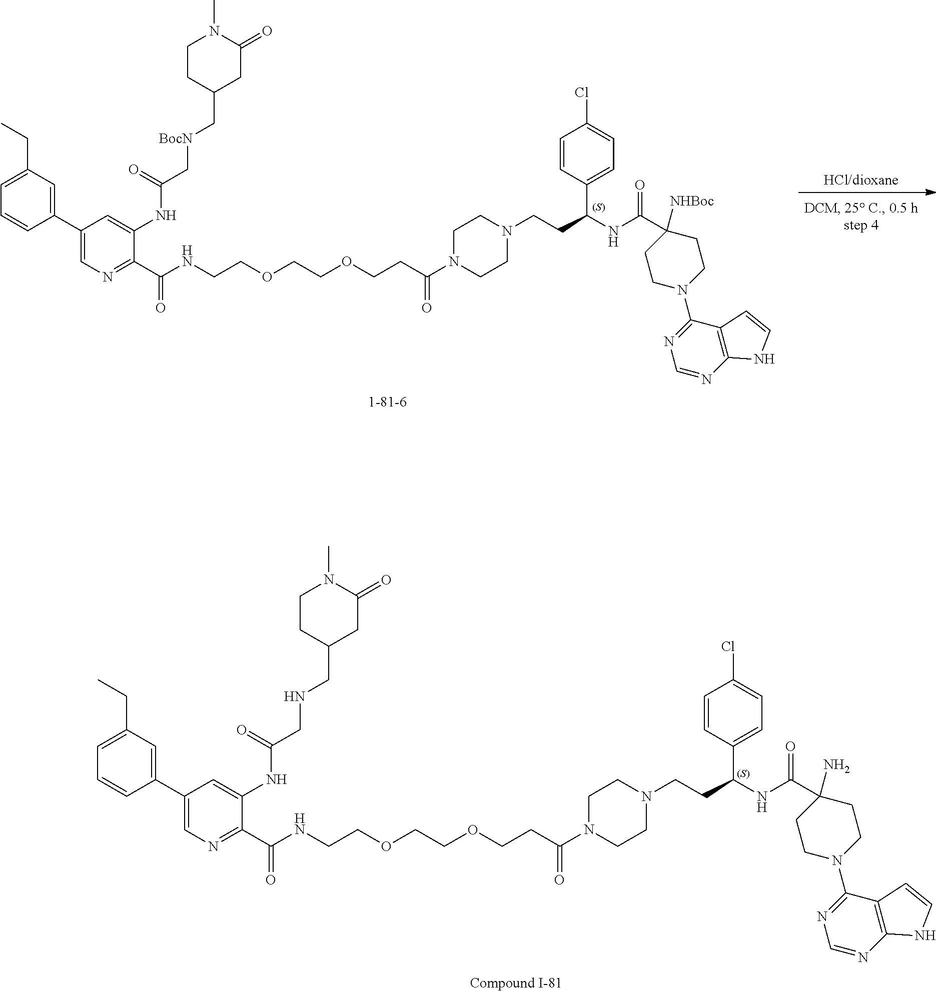

### Step 1: Synthesis of I-81-3

To a mixture of I-81-1 (100 mg, 190.62 μmol, 1 eq), EDCI (109.63 mg, 571.86 μmol, 3 eq) and HOAt (38.92 mg, 285.93 μmol, 1.5 eq) in DCM (1 mL) was added NMM (192.81 mg, 1.91 mmol, 209.57 μL, 10 eq), the mixture was stirred at 25° C. for 10 min. Then I-81-2 (182.28 mg, 800.60 μmol, 4.2 eq. HCl) was added. The mixture was stirred at 25° C. for 12 hr. Upon completion, the mixture was poured into water (3 mL) and extracted with DCM (2 mL×3). The combined organic layers were dried over anhydrous Na₂SO₄, filtered, and concentrated to give a residue. The residue was purified by flash silica gel chromatography (ISCO®; 4 g SepaFlash R: Silica Flash Column, Eluent of 0˜3% MeOH/Ethyl acetate gradient (a, 18 mL/min) and the eluent was concentrated to give I-81-3 (100 mg, 127.54 μmol, 66.91% yield, 89% purity) as colorless oil. LCMS (Method E): R^{t}=0.615 min, [M+H]⁺=698.4. ¹H NMR (400 MHz, CHLOROFORM-d) δ=12.58 (br d, J=10.8 Hz, 1H), 9.35 (br s, 1H), 8.64 (br s, 1H), 8.48 (br d, J=8.0 Hz, 1H), 7.52-7.28 (m, 4H), 3.82-3.78 (m, 2H), 3.71-3.66 (m, 10H), 3.62 (s, 2H), 3.34 (br d, J=4.0 Hz, 2H), 2.95 (s, 3H), 2.78-2.71 (m, 2H), 2.65-2.60 (m, 3H), 2.58-2.45 (m, 1H), 2.32-2.14 (m, 1H), 2.12-2.05 (m, 3H), 1.60-1.35 (m, 11H), 1.34-1.28 (m, 3H).

### Step 2: Synthesis of I-81-4

To a solution of I-81-3 (100 mg, 127.54 μmol, 1 eq) in THF (1 mL) and MeOH (0.5 mL) was added NaOH (20.41 mg, 510.16 μmol, 4 eq) in H2O (0.5 mL) and then the mixture was stirred at 25° C. for 1 h. Upon completion, the reaction mixture was adjusted to pH=6˜7 with 1N HCl and then concentrated under reduced pressure to give I-81-4 (85 mg, crude) as colorless gum. LCMS (Method E): R^{t}=0.573 min, [M+H]⁺=684.5.

### Step 3: Synthesis of I-81-6

To a mixture of I-81-4 (85 mg, 124.31 μmol, 1 eq), EDCI (71.49 mg, 372.92 μmol, 3 eq) and HOAt (16.92 mg, 124.31 μmol, 1 eq) in DCM (1 mL) was added NMM (62.87 mg, 621.54 μmol, 68.33 μL, 5 eq), the mixture was stirred at 25° C. for 10 min. Then I-81-5 (74.23 mg, 124.31 μmol, 1 eq) was added. The mixture was stirred at 25° C. for 3 hr. Upon completion, the mixture was poured into water (3 mL) and extracted with DCM (3 mL×3). The combined organic layers were dried over anhydrous Na₂SO₄, filtered, and concentrated to give a residue. The residue was purified by flash silica gel chromatography (ISCO®; 12 g SepaFlash® Silica Flash Column, Eluent of 0˜20% MeOH (5% NH3·H2O)/Ethyl acetate gradient @ 25 mL/min) and then repurified by prep-HPLC (column: YMC-Actus Triart C18 150×30 mm×7 um; mobile phase: [water (FA)-ACN]; gradient: 30%-60% B over 90 min), the eluent was concentrated to remove MeCN and then lyophilized. I-81-6 (38 mg, 30.09 μmol, 24.21% yield) was obtained as a white solid. LCMS (Method E): R^{t}=0.558 min, [M+H]⁺=1262.8. SFC: R^{t}=4.609 & 5.893 min.

### Step 4: Synthesis of I-81

To a mixture of I-81-6 (26 mg, 20.59 μmol, 1 eq) in DCM (1.2 mL) was added HCl/dioxane (4 M, 0.3 mL, 58.29 eq), the mixture was stirred at 25° C. for 0.5 hr. Upon completion, the reaction mixture was concentrated under reduced pressure to give the product. The product was combined with another batch for lyophilization. I-81 (32.38 mg, crude, HCl) as an off-white solid. LCMS (Method E): R^{t}=0.442 min, [M+H]⁺=1062.7. SFC: R^{t}=4.198 & 4.786 min. ¹H NMR (400 MHZ, METHANOL-d4) δ=9.23 (d, J=1.6 Hz, 1H), 8.65 (d, J=2.0 Hz, 1H), 8.38 (s, 1H), 7.57-7.48 (m, 2H), 7.47-7.38 (m, 4H), 7.38-7.27 (m, 3H), 7.00 (d, J=3.6 Hz, 1H), 5.05-5.01 (m, 1H), 4.68-4.50 (m, 3H), 4.34-4.17 (m, 3H), 4.05-3.87 (m, 2H), 3.78-3.72 (m, 2H), 3.72-3.34 (m, 14H), 3.24-2.98 (m, 6H), 2.96 (s, 3H), 2.89-2.68 (m, 5H), 2.67-2.45 (m, 3H), 2.45-2.29 (m, 2H), 2.28-2.17 (m, 2H), 2.17-2.01 (m, 2H), 1.76-1.59 (m, 1H), 1.31-1.27 (m, 3H).

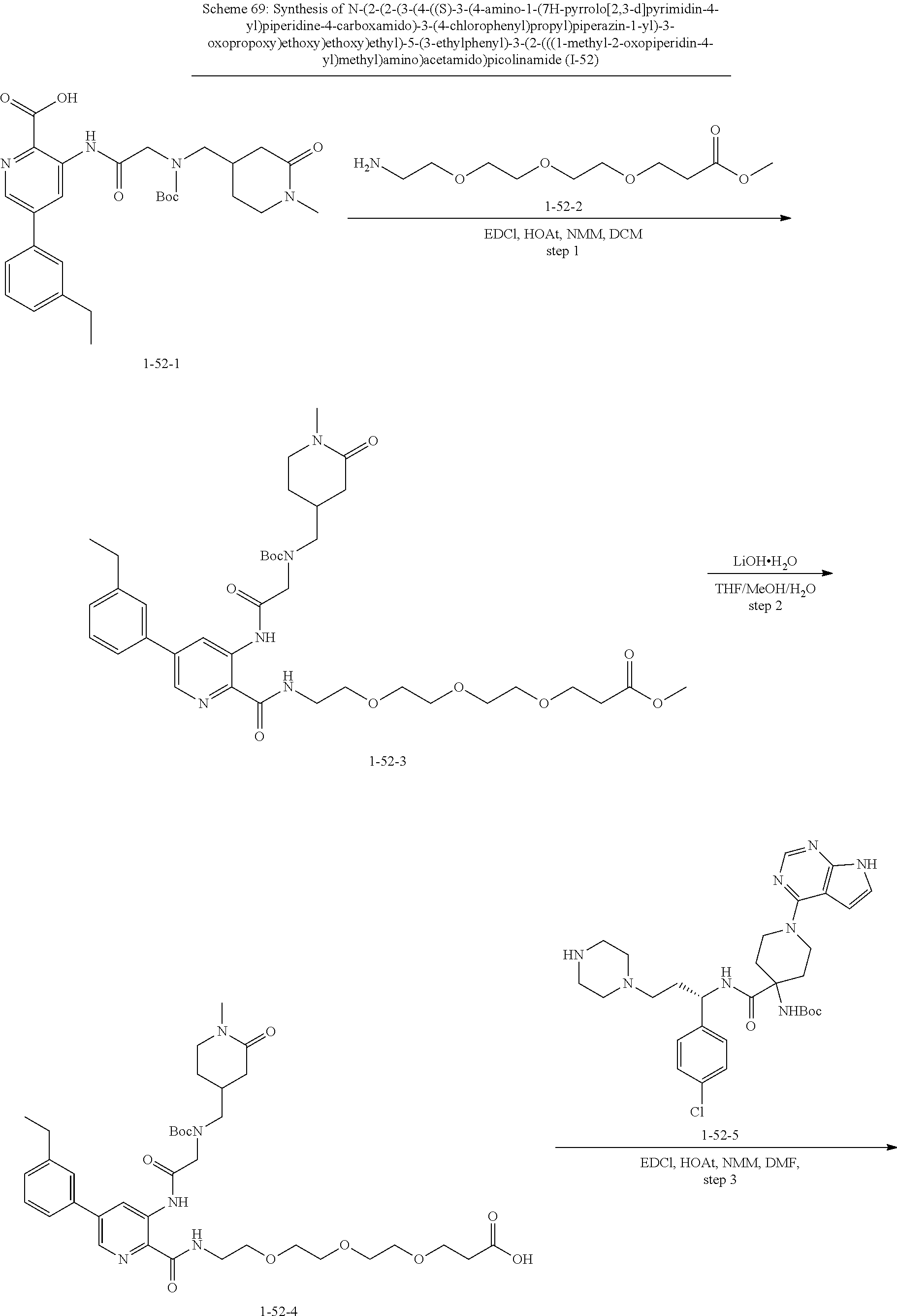

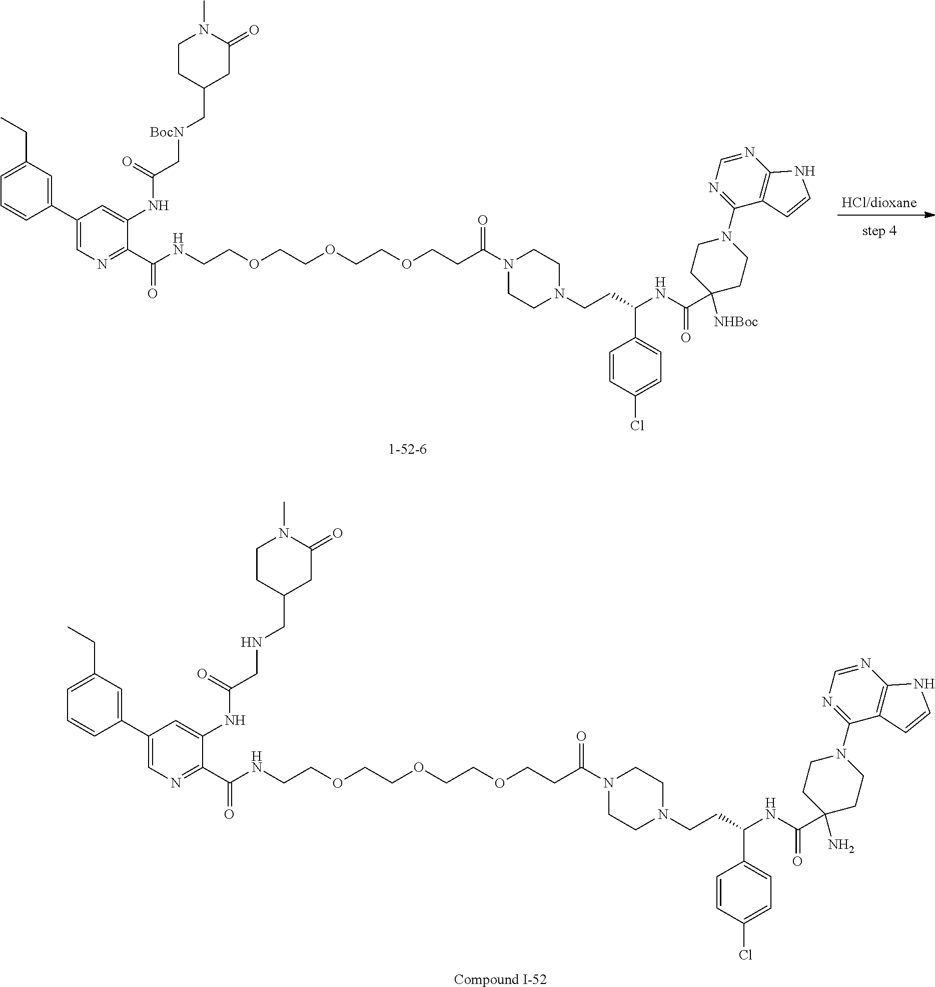 [SMILES: CCc1cccc(-c2cnc(C(=O)NCCOCCOCCOCCC(=O)N3CCN(CC[C@H](NC(=O)C4(NC(=O)OC(C)(C)C)CCN(c5ncnc6[nH]ccc56)CC4)c4ccc(Cl)cc4)CC3)c(NC(=O)CN(CC3CCN(C)C(=O)C3)C(=O)OC(C)(C)C)c2)c1 ; "HCl/dioxane" ; "1-52-6" ; CCc1cccc(-c2cnc(C(=O)NCCOCCOCCOCCC(=O)N3CCN(CC[C@H](NC(=O)C4(N)CCN(c5ncnc6[nH]ccc56)CC4)c4ccc(Cl)cc4)CC3)c(NC(=O)CNCC3CCN(C)C(=O)C3)c2)c1 ; "Compound I-52"]

### Step 1: Synthesis of I-52-3

To a solution of I-52-1 (100 mg, 190.62 μmol, 1 eq) and I-52-2 (51.80 mg, 190.62 μmol, 1 eq, HCl) in DCM (1 mL) was added EDCI (73.08 mg, 381.24 μmol, 2 eq), NMM (96.40 mg, 953.09 μmol, 104.79 μL, 5 eq) and HOAt (12.97 mg, 95.31 μmol, 0.5 eq). The mixture was stirred at 25° C. for 16 hrs. Upon completion, the reaction mixture was quenched with water (5 mL). The mixture was extracted with EA (5 mL×3) and dried over anhydrous Na₂SO₄. The mixture filtered, and the filtrate was concentrated under vacuum. The residue was purified by flash silica gel chromatography (ISCO®; 20 g SepaFlash® Silica Flash Column, Eluent of 80˜100% Ethyl acetate/Petroleum ether gradient @ 100 mL/min) to afford I-52-3 (80 mg, 87.35 μmol, 45.82% yield, 81% purity) as a white solid. LCMS (Method E): R^{t}=0.599 min, [M+H]⁺=742.6.

### Step 2: Synthesis of I-52-4

To a solution of I-52-3 (79 mg, 106.49 μmol, 1 eq) in THF (0.5 mL) was added LiOH·H₂O (13.41 mg, 319.46 μmol, 3 eq) in H₂O (0.5 mL) and MeOH (0.5 mL). The mixture was stirred at 25° C. for 1 hr. Upon completion, the reaction mixture was concentrated under the pressure to give I-52-4 (90 mg, 103.87 μmol, 97.54% yield, 84% purity) as a white solid. LCMS (Method E): R^{t}=0.562 min, [M+H]⁺=728.6.

### Step 3: Synthesis of I-52-6

To a solution of I-52-4 (90 mg, 102.89 μmol, 1 eq, Li) and I-52-5 (61.44 mg, 102.89 μmol, 1 eq) in DMF (1 mL) was added EDCI (59.17 mg, 308.66 μmol, 3 eq), NMM (52.03 mg, 514.44 μmol, 56.56μ L, 5 eq) and HOAt (14.00 mg, 102.89 μmol, 1 eq). The mixture was stirred at 25° C. for 4 hrs. Upon completion, the reaction mixture was quenched with water (5 mL). The mixture was extracted with EA (5 mL×3) and dried over anhydrous Na₂SO₄. The mixture filtered, and the filtrate was concentrated under vacuum. The residue was purified by prep-HPLC (column: Phenomenex luna C18 150×25 mm×10 um; mobile phase: [water (FA)-ACN]; gradient: 20%-50% B over 10 min) to afford I-52-6 (18 mg, 13.77 μmol, 13.39% yield, 100% purity) as a white solid. LCMS (Method E): R^{t}=0.543 min, [M+H]⁺=1329.7.

### Step 4: Synthesis of I-52

I-52-6 (18 mg, 13.77 μmol, 1 eq) was added in HCl/dioxane (0.5 mL) was stirred at 25° C. for 1 hr. Upon completion, the reaction mixture was concentrated and then dissolved in H₂O (1 mL). The solution was lyophilized to give I-52 (3.65 mg, 3.12 μmol, 22.63% yield, 97.63% purity; HCl) as a white solid. LCMS (Method E): R^{t}=0.451 min, [M+Na]⁺=1128.7. SFC: R^{t}=4.233 min, 4.760 min. ¹H NMR (400 MHz, METHANOL-d4) δ=9.23 (d, J=2.0 Hz, 1H), 8.64 (d, J=2.0 Hz. 1H), 8.39 (s. 1H), 7.58-7.29 (m, 9H), 7.00 (d, J=3.6 Hz. 1H), 5.08-4.97 (m, 2H), 4.69-4.53 (m, 3H), 4.32-4.15 (m, 3H), 4.02-3.88 (m, 2H), 3.75-3.52 (m, 18H), 3.49-3.42 (m, 2H), 3.35 (s, 2H), 3.19-2.95 (m, 8H), 2.88-2.28 (m, 10H), 2.26-2.01 (m, 4H), 1.76-1.59 (m, 1H), 1.32-1.27 (m, 3H).

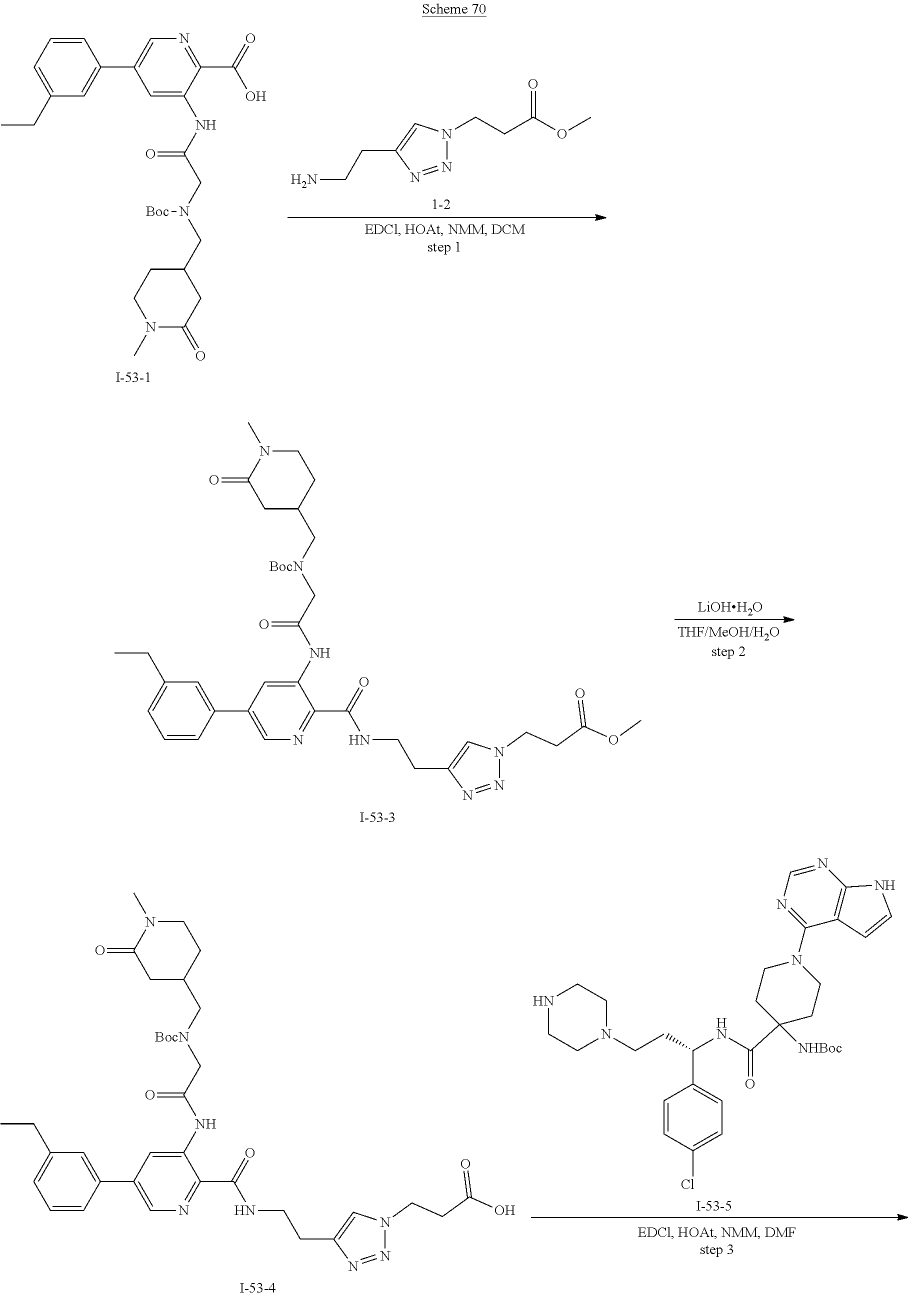 [SMILES: CCc1cccc(-c2cnc(C(=O)O)c(NC(=O)CN(CC3CCN(C)C(=O)C3)C(=O)OC(C)(C)C)c2)c1 ; COC(=O)CCn1cc(CCN)nn1 ; CCc1cccc(-c2cnc(C(=O)NCCc3cn(CCC(=O)OC)nn3)c(NC(=O)CN(CC3CCN(C)C(=O)C3)C(=O)OC(C)(C)C)c2)c1 ; "LiOH·H2O" ; CCc1cccc(-c2cnc(C(=O)NCCc3cn(CCC(=O)O)nn3)c(NC(=O)CN(CC3CCN(C)C(=O)C3)C(=O)OC(C)(C)C)c2)c1 ; CC(C)(C)OC(=O)NC1(C(=O)N[C@@H](CCN2CCNCC2)c2ccc(Cl)cc2)CCN(c2ncnc3[nH]ccc23)CC1]

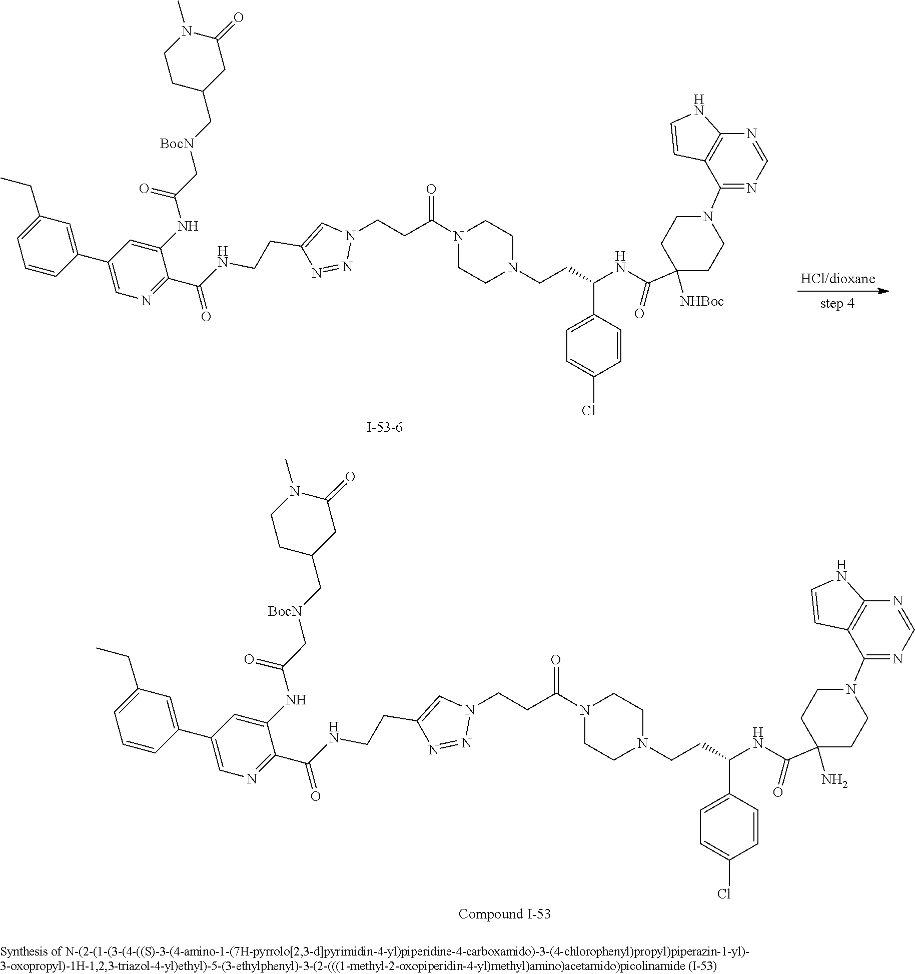 [SMILES: CCc1cccc(-c2cnc(C(=O)NCCc3cn(CCC(=O)N4CCN(CC[C@H](NC(=O)C5(NC(=O)OC(C)(C)C)CCN(c6ncnc7[nH]ccc67)CC5)c5ccc(Cl)cc5)CC4)nn3)c(NC(=O)CN(CC3CCN(C)C(=O)C3)C(=O)OC(C)(C)C)c2)c1 ; CCc1cccc(-c2cnc(C(=O)NCCc3cn(CCC(=O)N4CCN(CC[C@H](NC(=O)C5(N)CCN(c6ncnc7[nH]ccc67)CC5)c5ccc(Cl)cc5)CC4)nn3)c(NC(=O)CN(CC3CCN(C)C(=O)C3)C(=O)OC(C)(C)C)c2)c1]

### Step 1: Synthesis of I-53-3

A mixture of I-53-1 (100 mg, 190.62 μmol, 1 eq), I-53-2 (44.73 mg, 190.62 μmol, 1 eq, HCl), EDCI (73.08 mg, 381.24 μmol, 2 eq), HOAt (12.97 mg, 95.31 μmol, 0.5 eq) and NMM (96.40 mg, 953.09 μmol, 104.79 μL, 5 eq) in DCM (1 mL) was degassed and purged with N₂ for 3 times, and then the mixture was stirred at 25° C. for 16 hrs under N₂ atmosphere. Upon completion, the reaction mixture was quenched with water (5 mL). The mixture was extracted with EA (5 mL×3) and dried over anhydrous Na₂SO₄. The mixture filtered, and the filtrate was concentrated under vacuum. The residue was purified by flash silica gel chromatography (ISCO®; 20 g SepaFlash® Silica Flash Column, Eluent of 90˜100% Ethyl acetate/Petroleum ether gradient (@ 20 mL/min) to afford I-53-3 (80 mg, 113.51 μmol, 59.55% yield) as a white solid. LCMS (Method E): R^{t}=0.577 min, [M+H]⁺=705.5.

### Step 2: Synthesis of I-53-4

To a solution of I-53-3 (64 mg, 90.80 μmol, 1 eq) in THF (0.5 mL) was added LiOH·H₂O (11.43 mg, 272.41 μmol, 3 eq) in H₂O (0.5 mL) and MeOH (0.5 mL). The mixture was stirred at 25° C. for 1 hr. Upon completion, the reaction mixture was concentrated under the pressure to give I-53-4 (80 mg, 77.97 μmol, 85.86% yield, 68% purity, Li) as a white solid. LCMS (Method E): R^{t}=0.548 min, [M+H]⁺=691.5.

### Step 3: Synthesis of I-53-6

To a solution of I-53-4 (80 mg, 77.97 μmol, 1 eq, Li) and I-53-5 (46.56 mg, 77.97 μmol, 1 eq) in DMF (1 mL) was added EDCI (44.84 mg, 233.90 μmol, 3 eq), NMM (39.43 mg, 389.84 μmol, 42.86μ L, 5 eq) and HOAt (10.61 mg, 77.97 μmol, 1 eq). The mixture was stirred at 25° C. for 4 hrs. Upon completion, the reaction mixture was quenched with water (5 mL). The mixture was extracted with EA (5 mL×3) and dried over anhydrous Na₂SO₄. The mixture filtered, and the filtrate was concentrated under vacuum. The residue was purified by prep-HPLC (column: Phenomenex luna C18 150×25 mm×10 um; mobile phase: [water (FA)-ACN]; gradient: 20%-50% B over 10 min) to afford I-53-6 (22 mg, 17.32 μmol, 22.22% yield, 100% purity) as a white solid. LCMS (Method E): R^{t}=0.533 min, [M+H]⁺=1269.7.

### Step 4: Synthesis of I-53

To a solution of I-53-6 (22 mg, 17.32 μmol, 1 eq) in HCl/dioxane (0.5 mL) was stirred at 25° C. for 1 hr. Upon completion, the reaction mixture was concentrated under the pressure to reduce the solvent. The mixture was lyophilized to give I-53 (12.63 mg, 11.42 μmol, 65.91% yield, 100% purity, HCl salt) as a white solid. LCMS (Method E): R^{t}=0.414 min, [M+H]⁺=1069.7. SFC: R^{t}=4.562 min, 5.119 min. 1H NMR (400 MHZ, METHANOL-d4) δ=9.21 (d, J=1.6 Hz, 1H), 8.64 (d, J=1.6 Hz, 1H), 8.53-8.35 (m, 2H), 7.62-7.24 (m, 9H), 7.01 (d, J=3.6 Hz, 1H), 5.12-4.99 (m, 2H), 4.89-4.83 (m, 4H), 4.64-4.53 (m, 2H), 4.19 (d, J=7.6 Hz, 4H), 4.03-3.92 (m, 2H), 3.84-3.76 (m, 2H), 3.73-3.39 (m, 8H), 3.23-2.97 (m, 10H), 2.89-2.65 (m, 5H), 2.55-2.39 (m, 2H), 2.30-2.02 (m, 5H), 1.77-1.64 (m, 1H), 1.30-1.26 (m, 3H).

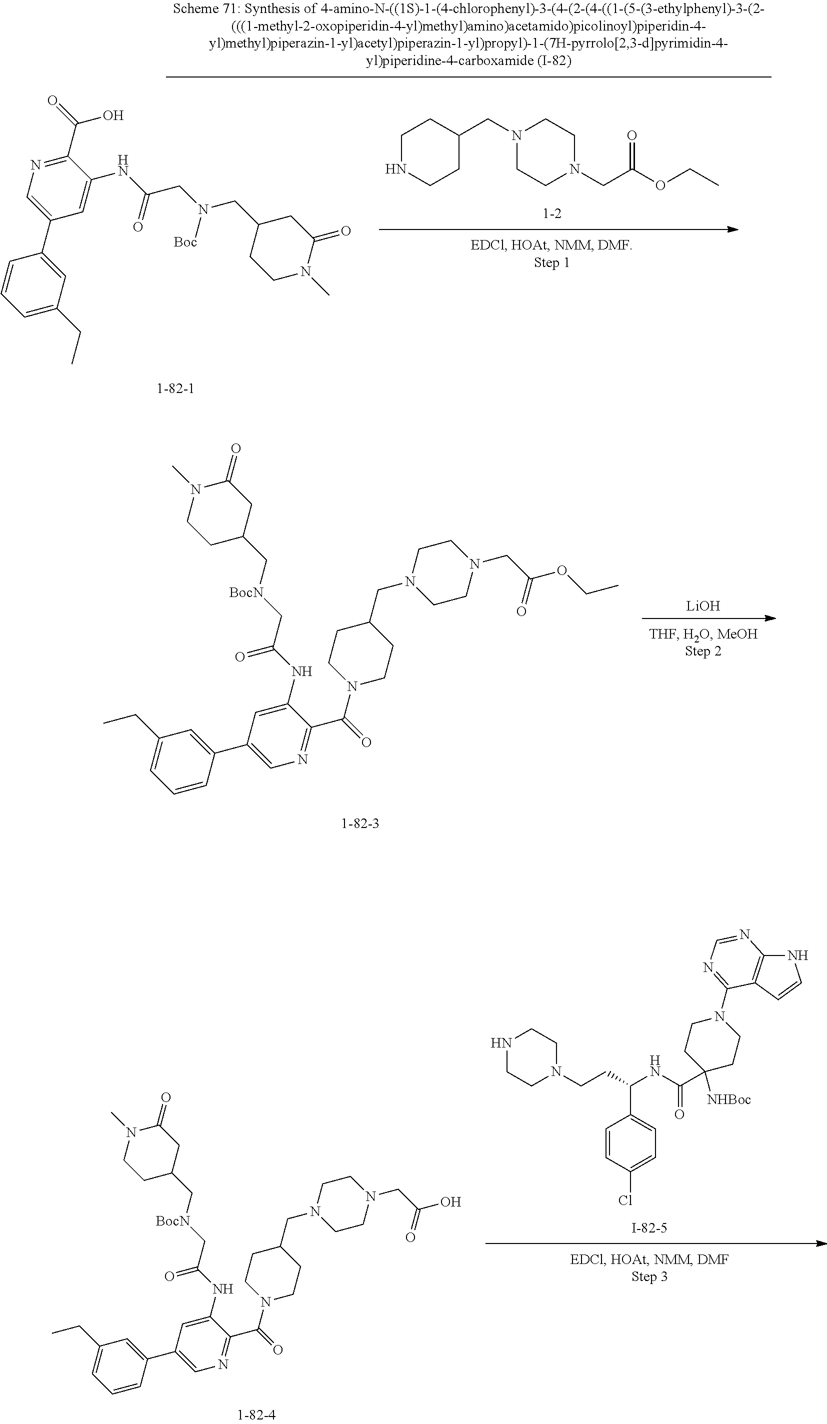

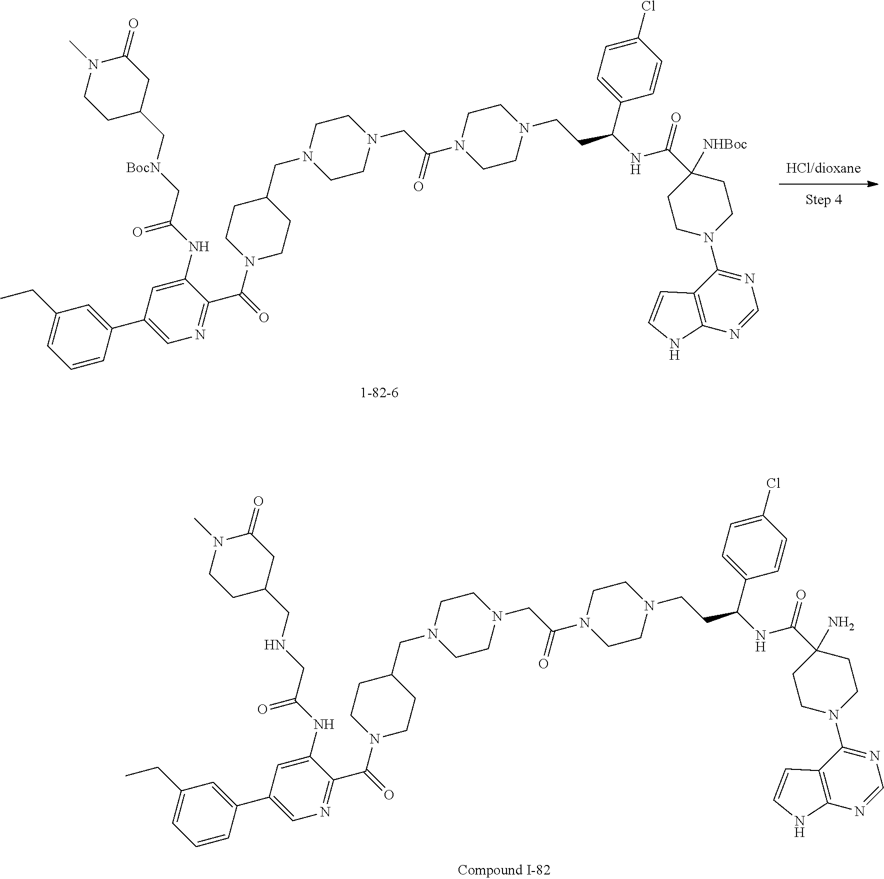 [SMILES: CCc1cccc(-c2cnc(C(=O)N3CCC(CN4CCN(CC(=O)N5CCN(CC[C@H](NC(=O)C6(NC(=O)OC(C)(C)C)CCN(c7ncnc8[nH]ccc78)CC6)c6ccc(Cl)cc6)CC5)CC4)CC3)c(NC(=O)CN(CC3CCN(C)C(=O)C3)C(=O)OC(C)(C)C)c2)c1 ; CCc1cccc(-c2cnc(C(=O)N3CCC(CN4CCN(CC(=O)N5CCN(CC[C@H](NC(=O)C6(N)CCN(c7ncnc8[nH]ccc78)CC6)c6ccc(Cl)cc6)CC5)CC4)CC3)c(NC(=O)CNCC3CCN(C)C(=O)C3)c2)c1]

### Step 1: Synthesis of I-82-3

To a solution of I-82-1 (80 mg, 152.50 μmol, 1 eq) in DMF (0.5 mL) was added EDCI (58.47 mg, 304.99 μmol, 2 eq), NMM (61.70 mg, 609.98 μmol, 67.06 μL, 4 eq), HOAt (41.51 mg, 304.99 μmol, 2 eq) and I-82-2 (61.62 mg, 228.74 μmol, 1.5 eq). The mixture was stirred at 25° C. for 12 hrs. The reaction mixture was quenched by addition H₂O 2 mL and extracted with EA (2 mL×3). The combined organic layers were washed with brine (20 mL×2), dried over Na₂SO₄, filtered, and concentrated under reduced pressure to give a residue. The residue was purified by prep-HPLC (basic condition) and the organic phase was concentrated under reduced pressure to remove acetonitrile, and the liquid was lyophilized to give I-82-3 (95 mg, 110.18 μmol, 72.25% yield, 90% purity) as a yellow oil. LCMS (Method G): R^{t}=0.694 min, [M+H]⁺=776.5.

### Step 2: Synthesis of I-82-4

To a solution of I-82-3 (95 mg, 122.43 μmol, 1 eq) in THF (0.5 mL) and MeOH (0.5 mL) was added LiOH·H₂O (15.41 mg, 367.28 μmol, 3 eq) in H₂O (0.5 mL). The mixture was stirred at 25° C. for 2 hr. The reaction mixture was quenched by addition HCl (1 M) to pH=5 and extracted with EA (10 mL×3). The combined organic layers were dried over Na₂SO₄, filtered, and concentrated under reduced pressure to give a residue. The crude product was used into the next step without further purification. I-82-4 (60 mg, 72.20 μmol, 58.97% yield, 90% purity) was obtained as a yellow oil. LCMS: R^{t}=0.453 min, [M−H]⁻=746.4.

### Step 3: Synthesis of I-82-6

To a solution of I-82-4 (60 mg, 80.22 μmol, 1 eq) in DMF (2 mL) was added HOAt (32.76 mg, 240.67 μmol, 3 eq), EDCI (46.14 mg, 240.67 μmol, 3 eq), NMM (40.57 mg, 401.11 μmol, 44.10 μL, 5 eq) and I-82-5 (71.86 mg, 120.33 μmol, 1.5 eq). The mixture was stirred at 25° C. for 12 hrs. The reaction mixture was quenched by addition H₂O 20 mL and extracted with EA (10 mL×3). The combined organic layers were washed with brine (10 mL×2), dried over Na₂SO₄, filtered, and concentrated under reduced pressure to give a residue. The crude product was used into the next step without further purification. I-82-6 (145 mg, crude) was obtained as a yellow oil. LCMS (Method G): R^{t}=0.741 min, [M+H]⁺=1326.9.

### Step 4: Synthesis of I-82

To a solution of I-82-6 (145 mg, 109.26 μmol, 1 eq) was added HCl/dioxane (4 M, 27.32 μL, 1 eq). The mixture was stirred at 25° C. for 1 hr. The reaction mixture was quenched by addition H₂O 20 mL and extracted with EA (10 mL×3). The combined organic layers were washed with brine mL (10 mL×2), dried over Na₂SO₄, filtered, and concentrated under reduced pressure to give a residue. The residue was purified by prep-HPLC (column: C18 150×30 mm; mobile phase: [water (FA)-ACN]; gradient: 0%-30% B over 12 min). I-82 (13.62 mg, 12.09 μmol, 11.06% yield, 100% purity) was obtained as a yellow solid. LCMS (Method E): R^{t}=0.384 min, [M+H]⁺=1127.1. SFC: R^{t}=2.858 & 4.222 min. ¹H NMR (400 MHZ, METHANOL-d4) δ=8.83-8.76 (m, 1H), 8.62 (d, J=1.6 Hz, 1H), 8.54-8.30 (m, 1H), 8.17 (s, 1H), 7.59-7.49 (m, 2H), 7.45 (t, J=7.6 Hz, 1H), 7.39-7.31 (m, 5H), 7.17 (d, J=3.6 Hz, 1H), 6.66 (d, J=3.6 Hz, 1H), 5.00 (br d, J=6.4 Hz, 2H), 4.86 (br d, J=2.8 Hz, 2H), 4.71 (br d, J=13.6 Hz, 1H), 4.65-4.55 (m, 2H), 3.85 (br d, J=13.2 Hz, 1H), 3.60 (br d, J=17.6 Hz, 6H), 3.54-3.39 (m, 6H), 3.19 (br t, J=13.2 Hz, 1H), 2.96 (s, 4H), 2.90-2.72 (m, 9H), 2.68 (br s, 2H), 2.62-2.35 (m, 9H), 2.27 (br d, J=11.6 Hz, 2H), 2.19-1.92 (m, 7H), 1.83-1.68 (m, 3H), 1.68-1.56 (m, 1H), 1.31 (t, J=7.6 Hz, 3H).

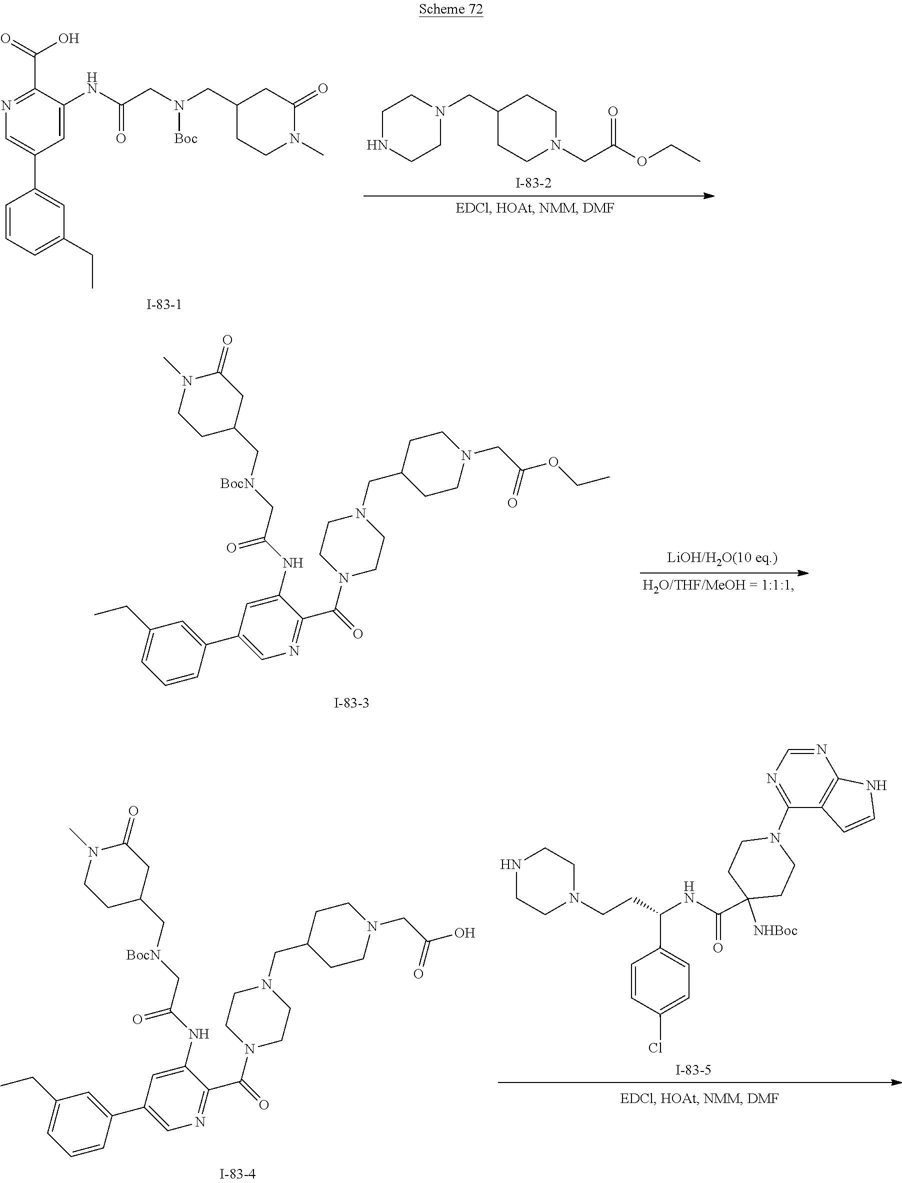 [SMILES: CCc1cccc(-c2cnc(C(=O)O)c(NC(=O)CN(CC3CCN(C)C(=O)C3)C(=O)OC(C)(C)C)c2)c1 ; CCOC(=O)CN1CCC(CN2CCNCC2)CC1 ; CCOC(=O)CN1CCC(CN2CCN(C(=O)c3ncc(-c4cccc(CC)c4)cc3NC(=O)CN(CC3CCN(C)C(=O)C3)C(=O)OC(C)(C)C)CC2)CC1 ; CCc1cccc(-c2cnc(C(=O)N3CCN(CC4CCN(CC(=O)O)CC4)CC3)c(NC(=O)CN(CC3CCN(C)C(=O)C3)C(=O)OC(C)(C)C)c2)c1 ; CC(C)(C)OC(=O)NC1(C(=O)N[C@@H](CCN2CCNCC2)c2ccc(Cl)cc2)CCN(c2ncnc3[nH]ccc23)CC1]

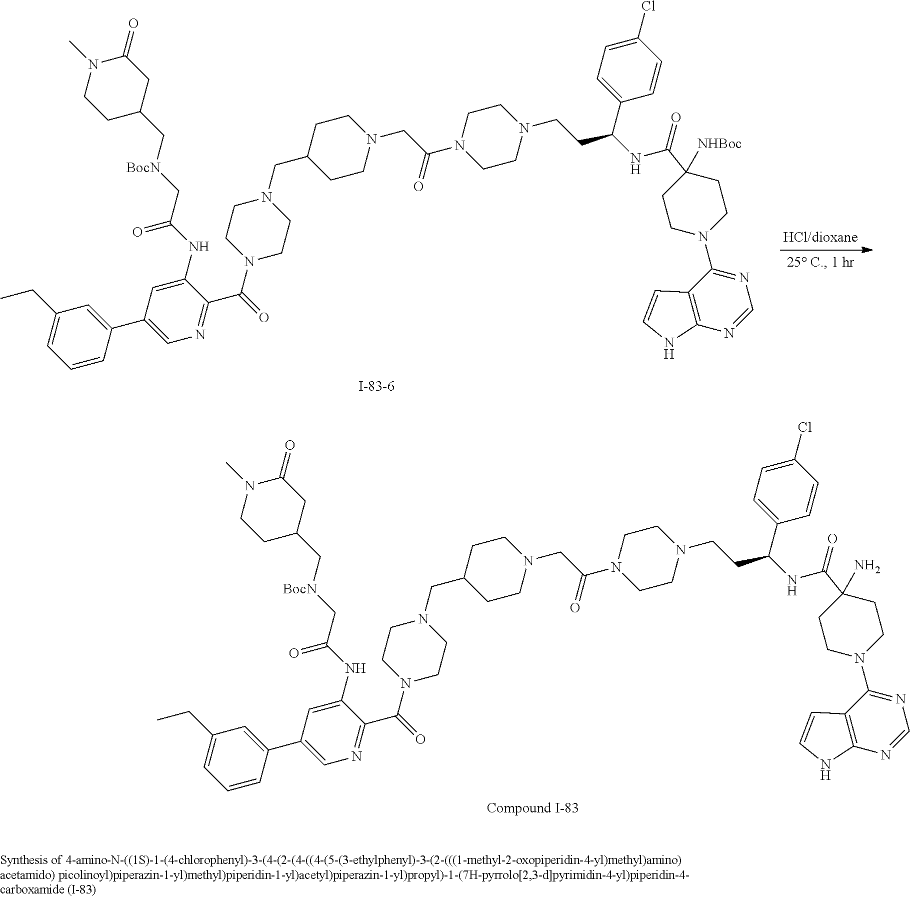 [SMILES: CCc1cccc(-c2cnc(C(=O)N3CCN(CC4CCN(CC(=O)N5CCN(CC[C@H](NC(=O)C6(NC(=O)OC(C)(C)C)CCN(c7ncnc8[nH]ccc78)CC6)c6ccc(Cl)cc6)CC5)CC4)CC3)c(NC(=O)CN(CC3CCN(C)C(=O)C3)C(=O)OC(C)(C)C)c2)c1 ; CCc1cccc(-c2cnc(C(=O)N3CCN(CC4CCN(CC(=O)N5CCN(CC[C@H](NC(=O)C6(N)CCN(c7ncnc8[nH]ccc78)CC6)c6ccc(Cl)cc6)CC5)CC4)CC3)c(NC(=O)CN(CC3CCN(C)C(=O)C3)C(=O)OC(C)(C)C)c2)c1]

### Step 1: Synthesis of I-83-3

To a solution of I-83-1 (120 mg, 228.74 μmol, 1 eq), I-83-2 (123.24 mg, 457.49 μmol, 2 eq) in DMF (1 mL) was added EDCI (131.55 mg, 686.23 μmol, 3 eq), NMM (115.69 mg, 1.14 mmol, 125.75 μL, 5 eq) and HOAt (93.40 mg, 686.23 μmol, 3 eq). The mixture was stirred at 25° C. for 16 hr. The reaction mixture was washed with water (5 mL) and extracted with EA (4 mL×2). The combined organic phase was washed with brine (5 mL), dried with anhydrous Na₂SO₄, filtered, and concentrated in vacuum to give a residue. The residue was purified by reversed phase HPLC (0.1% FA condition) to give I-83-3 (45 mg, 49.29 μmol, 21.55% yield, 85% purity) as a white solid. LCMS (Method G): R^{t}=0.687 min, [M+H]⁺=776.4.

### Step 2: Synthesis of I-83-4

To a solution of I-83-3 (45 mg, 57.99 μmol, 1 eq) in MeOH (0.5 mL), THF (0.5 mL), H₂O (0.5 mL) was added LiOH·H₂O (24.34 mg, 579.92 μmol, 10 eq). The mixture was stirred at 25° C. for 1 hr. Upon completion, the reaction mixture was concentrated under reduced pressure to give a residue. The residue was purified by reversed-phase HPLC (0.1% FA condition) to give I-83-4 (35 mg, 45.86 μmol, 79.08% yield, 98% purity) as a white solid. LCMS (Method G): R^{t}=0.503 min, [M+H]⁺=748.3.

### Step 3: Synthesis of I-83-6

To a solution of I-83-4 (25 mg, 33.43 μmol, 1 eq), I-83-5 (29.94 mg, 50.14 μmol, 1.5 eq) in DMF (1 mL) was added EDCI (19.22 mg, 100.28 μmol, 3 eq), NMM (16.90 mg, 167.13 μmol, 18.37 μL, 5 eq) and HOAt (13.65 mg, 100.28 μmol, 3 eq). The mixture was stirred at 25° C. for 16 hr. Upon completion, the reaction mixture was diluted with 0.5 mL DMF and filtered. The filtrate was purified by reversed-phase HPLC (0.1% FA condition) to give a yellow oil. I-83-6 (30 mg, 11.76 μmol, 35.17% yield, 52% purity) as a yellow solid. LCMS (Method G): R^{t}=0.772 min, [M+H]⁺=1327.0.

### Step 7: Synthesis of 1-83

To a solution of I-83-6 (30 mg, 22.61 μmol, 1 eq) in dioxane (0.3 mL) was added HCl/dioxane (4 M, 0.2 mL, 35.39 eq). The mixture was stirred at 25° C. for 0.5 hr. Upon completion, the reaction mixture was concentrated under reduced pressure to give a yellow solid. The yellow solid was purified by prep-HPLC (column: Phenomenex luna C18 150×25 mm×10 um; mobile phase: [water (HCl)-ACN]; gradient: 1%-25% B over 10 min) to give I-83 (2.59 mg, 2.18 μmol, 9.63% yield, 94.689% purity) as a yellow solid. LCMS (Method G): R^{t}=0.672 min, [M+H]⁺=1126.8. ¹H NMR (400 MHZ, METHANOL-d4) δ=8.73 (d, J=1.6 Hz, 1H), 8.39-8.33 (m, 1H), 8.21 (d, J=1.6 Hz, 1H), 7.54 (s, 2H), 7.44 (br d, J=11.6 Hz, 3H), 7.39-7.33 (m, 4H), 6.95 (d, J=3.6 Hz, 1H), 5.14-4.98 (m, 2H), 4.70-4.56 (m, 3H), 4.46-4.33 (m, 2H), 4.13 (s, 3H), 3.94-3.78 (m, 4H), 3.77-3.66 (m, 3H), 3.44 (br d, J=5.2 Hz, 10H), 3.27-3.06 (m, 6H), 2.95 (s, 3H), 2.81-2.02 (m, 17H), 1.84-1.60 (m, 3H), 1.29 (t, J=7.6 Hz, 5H).

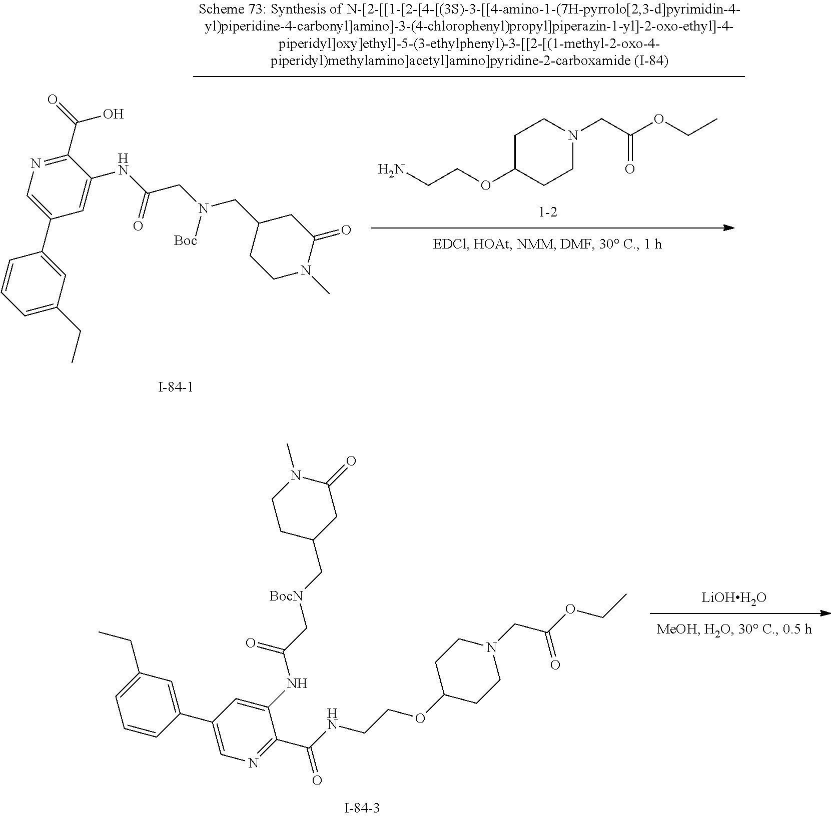 [SMILES: CCc1cccc(-c2cnc(C(=O)O)c(NC(=O)CN(CC3CCN(C)C(=O)C3)C(=O)OC(C)(C)C)c2)c1 ; CCOC(=O)CN1CCC(OCCN)CC1 ; CCOC(=O)CN1CCC(OCCNC(=O)c2ncc(-c3cccc(CC)c3)cc2NC(=O)CN(CC2CCN(C)C(=O)C2)C(=O)OC(C)(C)C)CC1 ; "LiOH·H2O"]

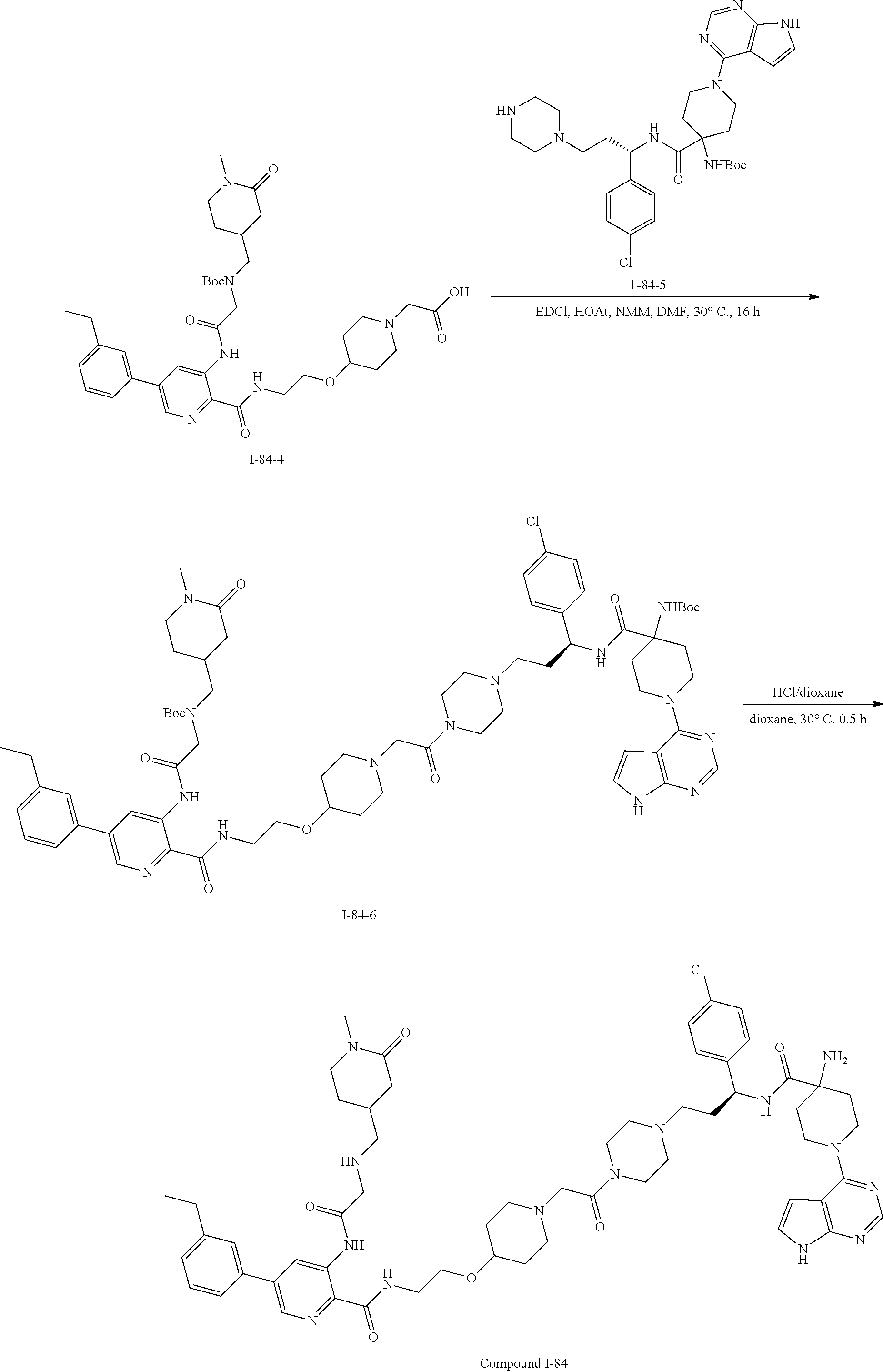

### Step 1: Synthesis of I-84-3

To a solution of I-84-1 (80 mg, 152.50 μmol. 1 eq) and I-84-2 (52.68 mg, 228.74 μmol, 1.5 eq) in DMF (1.5 mL) was added EDCI (87.70 mg, 457.49 μmol, 3 eq). HOAt (62.27 mg, 457.49 μmol, 3 eq) and NMM (77.12 mg, 762.48 μmol, 83.83 μL, 5 eq). The mixture was stirred at 30° C. for 1 h. Upon completion, the mixture was concentrated. The crude product was purified by reversed-phase HPLC (0.1% NH₃·H₂O: ACN: 0-60%). I-84-3 (50 mg, 65.82 μmol, 43.16% yield, 97% purity) was obtained as a yellow oil. LCMS (Method G): R^{t}=0.722 min, [M+H]⁺=737.3.

### Step 2: Synthesis of I-84-4

To a solution of I-84-3 (50 mg, 67.85 μmol, 1 eq) in MeOH (1 mL) was added LiOH·H₂O (14.24 mg, 339.26 μmol, 5 eq) in H₂O (1 mL). The mixture was stirred at 30° C. for 0.5 hr. Upon completion, the reaction mixture was adjusted to pH˜6 by HCl (1 M) and extracted by EtOAc (2 mL×4). The combined organic layers were dried over Na₂SO₄, filtered, and concentrated under reduced pressure. I-84-4 (42 mg, 55.10 μmol, 81.21% yield, 93% purity) was obtained as a white solid. LCMS (Method E): R^{t}=0.499 min, [M+H]⁺=709.4.

### Step 3: Synthesis of I-84-6

To a solution of I-84-4 (40 mg, 56.43 μmol, 1 eq) and I-84-5 (50.55 mg, 84.65 μmol, 1.5 eq) in DMF (1 mL) was added EDCI (32.45 mg, 169.29 μmol, 3 eq), HOAt (23.04 mg, 169.29 μmol, 3 eq) and NMM (28.54 mg, 282.15 μmol, 31.02 μL, 5 eq). The mixture was stirred at 30° C. for 16 h. Upon completion, the mixture was concentrated. The crude product was purified by reversed-phase HPLC (0.1% NH₃·H₂O:ACN: 0-60%). I-84-6 (35 mg, 19.02 μmol, 33.71% yield, 70% purity) was obtained as a yellow solid. LCMS (Method C): R^{t}=1.111 min, [M+H]⁺=1288.0.

### Step 4: Synthesis of I-84

To a solution of I-84-6 (35 mg, 27.17 μmol, 1 eq) in dioxane (0.5 mL) was added HCl/dioxane (4 M, 0.5 mL). The mixture was stirred at 30° C. for 0.5 h. Upon completion, the mixture was concentrated. The crude was purified by Prep-HPLC (column: Phenomenex luna C18 150×25 mm×10 um; mobile phase: [water (HCl)-ACN]; gradient: 1%-30% B over 10 min). I-84 (13.49 mg, 12.28 μmol, 45.18% yield, 99% purity) was obtained as a white solid. LCMS (Method B): R^{t}=0.488 min, [M+H]⁺=1087.9. ¹H NMR (400 MHZ, METHANOL-d4) δ=9.23 (s, 1H), 8.70-8.61 (m, 1H), 8.38 (s, 1H), 7.56-7.48 (m, 2H), 7.47-7.39 (m, 4H), 7.38-7.29 (m, 3H), 7.01 (d, J=3.6 Hz, 1H), 5.06 (br dd, J=4.8, 9.6 Hz, 1H), 4.67-4.35 (m, 5H), 4.25 (s, 2H), 4.08-3.80 (m, 4H), 3.78-3.54 (m, 8H), 3.52-3.32 (m, 7H), 3.27-3.09 (m, 5H), 2.95 (s, 3H), 2.89-2.67 (m, 4H), 2.66-2.46 (m, 2H), 2.45-2.04 (m, 9H), 1.96-1.81 (m, 1H), 1.77-1.61 (m, 1H), 1.29 (t, J=7.6 Hz, 3H), SFC: R^{t}=4.475 min, 5.241 min.

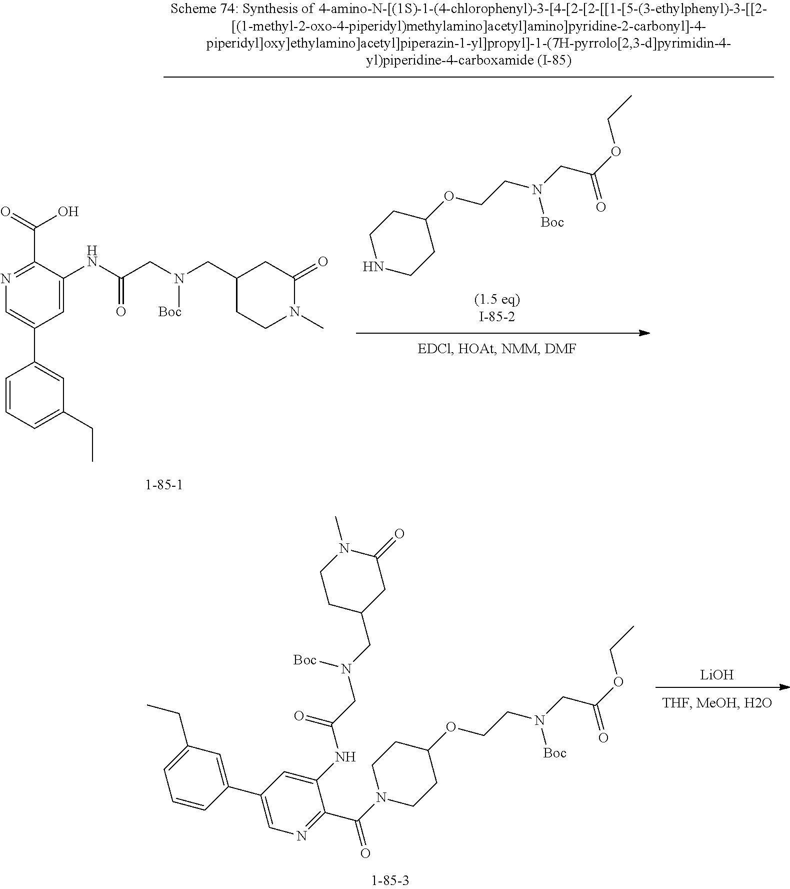 [SMILES: CCc1cccc(-c2cnc(C(=O)O)c(NC(=O)CN(CC3CCN(C)C(=O)C3)C(=O)OC(C)(C)C)c2)c1 ; CCOC(=O)CN(CCOC1CCNCC1)C(=O)OC(C)(C)C ; CCOC(=O)CN(CCOC1CCN(C(=O)c2ncc(-c3cccc(CC)c3)cc2NC(=O)CN(CC2CCN(C)C(=O)C2)C(=O)OC(C)(C)C)CC1)C(=O)OC(C)(C)C ; "LiOH"]

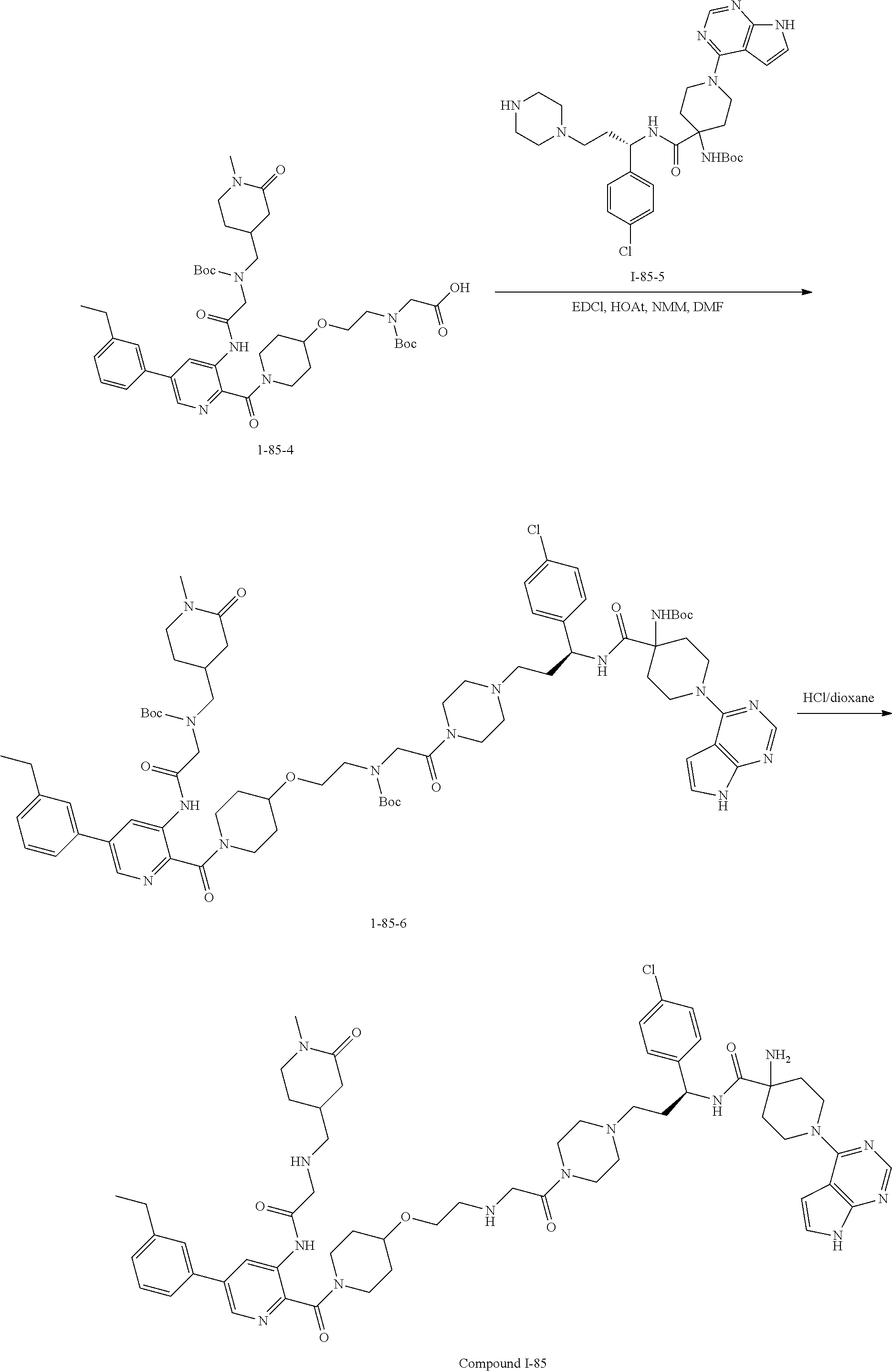

### Step 1: Synthesis of I-85-3

To a solution of I-85-1 (60 mg, 114.37 μmol, 1 eq) and EDCI (65.77 mg, 343.11 μmol, 3 eq) and HOAt (46.70 mg, 343.11 μmol, 3 eq) in DMF (1 mL) was added I-85-2 (56.69 mg, 171.56 μmol, 1.5 eq) and NMM (57.84 mg, 571.86 μmol, 62.87 μL, 5 eq), the mixture was stirred at 25° C. for 2 hr. Upon completion, the residue was diluted with H₂O (20 mL) and was extracted with ethyl acetate (25 mL×2). The combined organic phase was washed with brine (10 mL), dried with anhydrous Na₂SO₄, filtered, and concentrated in vacuum. The crude product was purified by reverse-phase HPLC (0.1% NH₃·H₂O). I-85-3 (90 mg, 104.30 μmol, 91.19% yield, 97% purity) was obtained as a brown solid. LCMS (Method G): R^{t}=0.773 min, [M+H]⁺=873.4.

### Step 2: Synthesis of I-85-4

To a solution of I-85-3 (80 mg, 95.58 μmol, 1 eq) in MeOH (0.5 mL), H₂O (0.5 mL) and THF (0.5 mL) was added LiOH·H₂O (12.03 mg, 286.73 μmol, 3 eq), the mixture was stirred at 25° C. for 2 hrs. Upon completion, the reaction solution was acidified with HCl (1M) to pH=5 and the aqueous phase was extracted with ethyl acetate (20 mL×3). The combined organic phase was washed with brine (10 mL×2), dried with anhydrous Na₂SO₄, filtered, and concentrated in vacuum. The crude product was used in the next step directly. I-85-4 (90 mg, 90.12 μmol, 94.29% yield, 81% purity) was obtained as a yellow solid. LCMS (Method G): R^{t}=0.436 min, [M+H]⁺=809.3.

### Step 3: Synthesis of I-85-6

To a solution of I-85-4 (80 mg, 98.89 μmol, 1 eq), EDCI (56.87 mg, 296.68 μmol, 3 eq) and HOAt (40.38 mg, 296.68 μmol, 3 eq) in DMF (1 mL) was added I-85-5 (88.58 mg, 148.34 μmol, 1.5 eq) and NMM (50.01 mg, 494.46 μmol, 54.36 μL, 5 eq), the mixture was stirred at 25° C. for 2 hrs. Upon completion, the residue was diluted with H₂O (20 mL) and was extracted with ethyl acetate (15 mL×2). The combined organic phase was washed with brine (10 mL), dried with anhydrous Na₂SO₄, filtered, and concentrated in vacuum. The crude product was used in the next step directly. I-85-6 (100 mg, 41.42 μmol, 41.89% yield, 57.5% purity) was obtained as a yellow solid. LCMS (Method E): R^{t}=0.576 min, [M+H]⁺=695.0.

### Step 4: Synthesis of I-85

To a solution of I-85-6 (100 mg, 72.04 μmol, 1 eq) in dioxane (1 mL) was added HCl/dioxane (2 mL), the mixture was stirred at 25° C. for 0.5 hr. Upon completion, the reaction mixture was concentrated under reduced pressure to give the crude product. The crude product was purified by Prep-HPLC (column: Phenomenex Luna C18 150×25 mm×10 um; mobile phase: [water (TFA)-ACN]; gradient: 10%-40% B over 9 min) to give I-85 (17.42 mg, 14.89 μmol, 20.67% yield, 93% purity) as a white solid. LCMS (Method G): R^{t}=0.642 min, [M+H]⁺=1087.8. SFC: R^{t}=0.439 min. ¹H NMR (400 MHZ, METHANOL-d4) 8=8.68 (d, J=2.0 Hz, 1H), 8.35 (s, 1H), 8.31 (dd, J=2.0, 3.6 Hz, 1H), 7.54-7.51 (m, 1H), 7.49 (s, 1H), 7.47-7.41 (m, 1H), 7.37-7.33 (m, 6H), 6.88 (d, J=3.6 Hz, 1H), 5.06-5.01 (m, 1H), 4.67-4.58 (m, 2H), 4.19 (s, 2H), 4.13-4.05 (m, 3H), 3.89-3.66 (m, 10H), 3.59-3.33 (m, 8H), 3.28 (m, 3H), 3.16-3.08 (m, 3H), 2.95 (s, 3H), 2.75 (d, J=7.6 Hz, 2H), 2.67-2.52 (m, 3H), 2.40-2.24 (m, 3H), 2.17 (m, 2H), 2.11-1.99 (m, 4H), 1.78 (m, 2H), 1.72-1.59 (m, 1H), 1.29 (t, J=7.6 Hz, 3H).

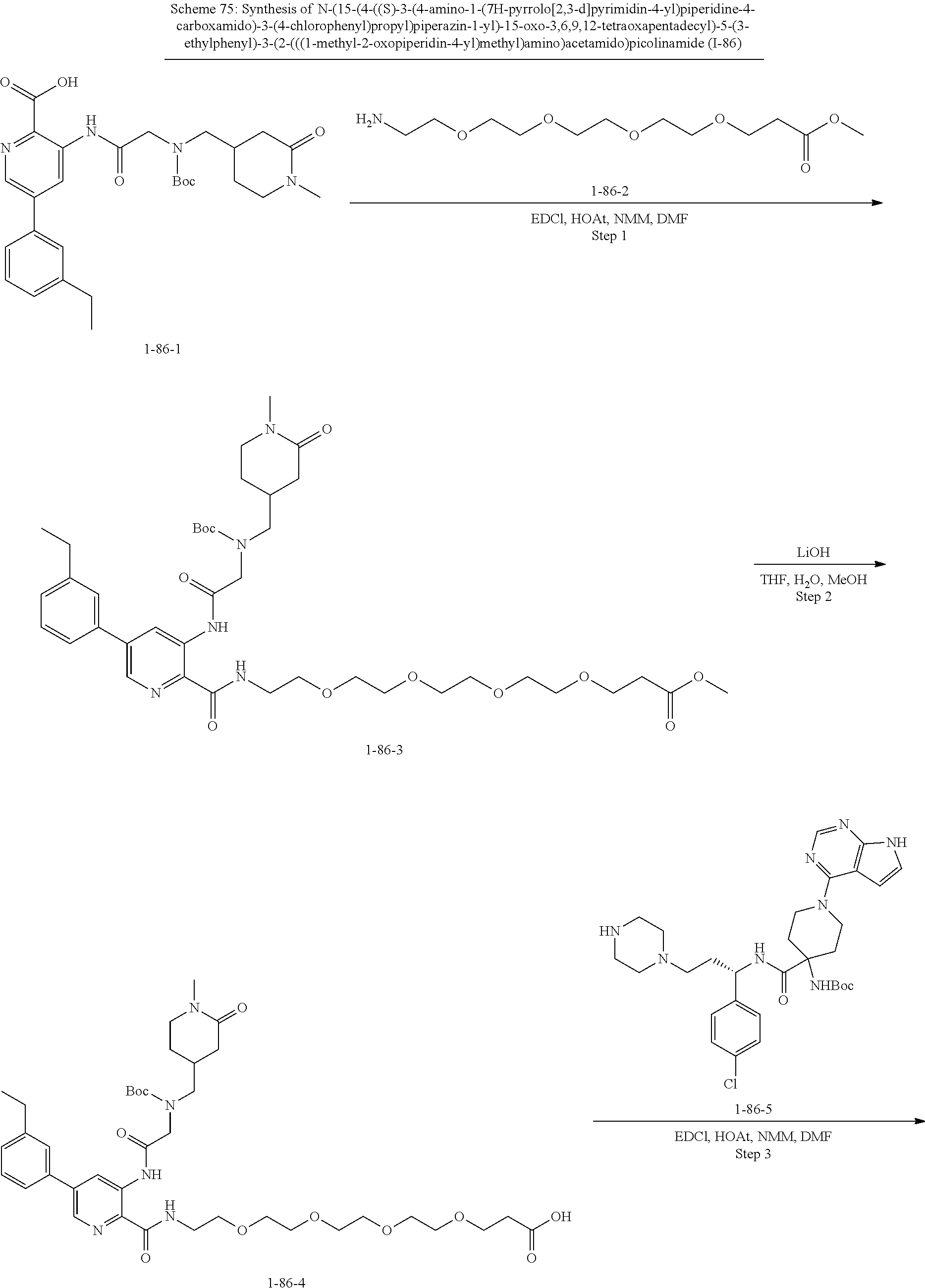

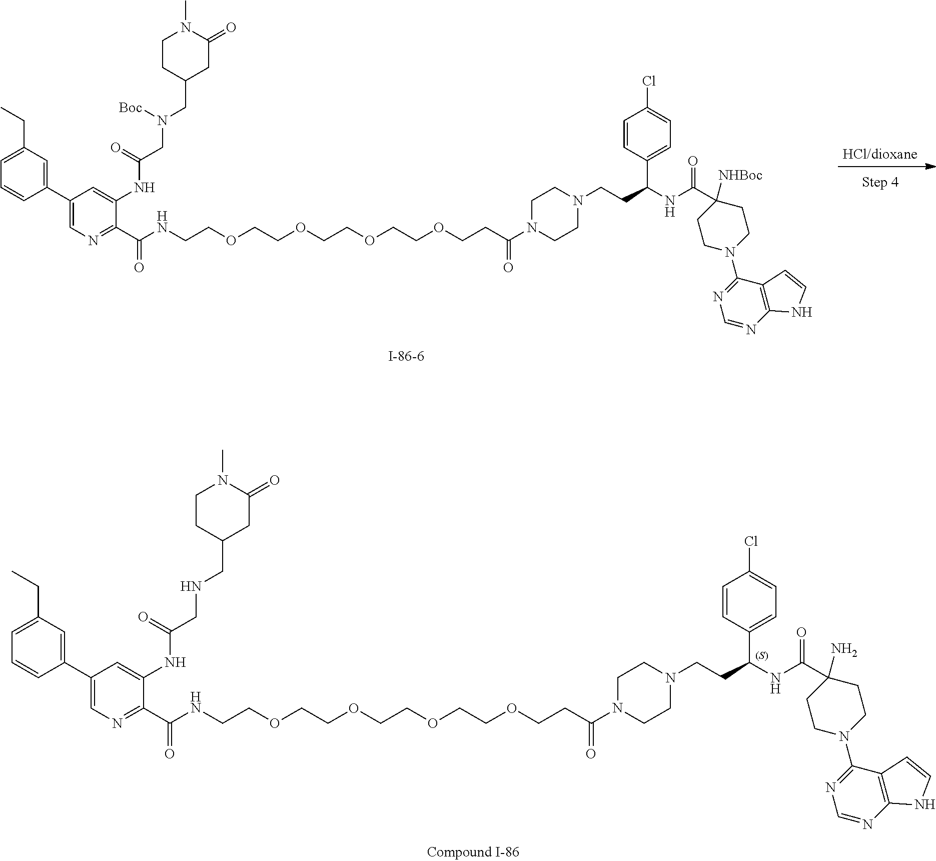

### Step 1: Synthesis of I-86-3

To a solution of I-86-1 (80 mg, 152.50 μmol, 1 eq) in DMF (1 mL) was added EDCI (58.47 mg, 304.99 μmol, 2 eq), NMM (61.70 mg, 609.98 μmol, 67.06 μL, 4 eq), HOBt (41.21 mg, 304.99 μmol, 2 eq) and I-86-2 (63.89 mg, 228.74 μmol, 1.5 eq). The mixture was stirred at 25° C. for 12 hrs. The reaction mixture was quenched by addition H₂O (10 mL) and extracted with EA (10 mL×3). The combined organic layers were washed with brine (10 mL×2), dried over Na₂SO₄, filtered, and concentrated under reduced pressure to give a residue. The residue was dissolved in DMF (0.5 mL) and purified by prep-HPLC (basic condition) and the organic phase was concentrated under reduced pressure to remove acetonitrile, and the liquid was lyophilized to give I-86-3 (45 mg, 51.53 μmol, 33.79% yield, 90% purity) as colorless oil. LCMS (Method G): R^{t}=0.714 min, [M+H]⁺=786.5.

### Step 2: Synthesis of I-86-4

To a solution of I-86-3 (45 mg, 57.26 μmol, 1 eq) in THF (0.4 mL) MeOH (0.4 mL) was added LiOH·H₂O (7.21 mg, 171.77 μmol, 3 eq) in H₂O (0.4 mL). The mixture was stirred at 25° C. for 2 hrs. The reaction mixture was quenched by addition HCl (1 M) to pH=5 and extracted with EA (10 mL×3). The combined organic layers were dried over Na₂SO₄, filtered, and concentrated under reduced pressure to give a residue. The crude product was used into the next step without further purification. I-86-4 (45 mg, 52.47 μmol, 91.64% yield, 90% purity) was obtained as a yellow oil. LCMS (Method G): R^{t}=0.449 min, [M+H]⁺=770.4.

### Step 3: Synthesis of I-86-6

To a solution of I-86-4 (45 mg, 58.30 μmol, 1 eq) in DMF (1 mL) was added HOAt (23.81 mg, 174.89 μmol, 3 eq), NMM (29.48 mg, 291.49 μmol, 32.05 μL, 5 eq), EDCI (33.53 mg, 174.89 μmol, 3 eq) and I-86-5 (52.22 mg, 87.45 μmol, 1.5 eq). The mixture was stirred at 25° C. for 12 hrs. The reaction mixture was quenched by addition H₂O 20 mL and extracted with EA (10 mL×3). The combined organic layers were washed with brine (10 mL×2), dried over Na₂SO₄, filtered, and concentrated under reduced pressure to give a residue. The crude product was used into the next step without further purification. I-86-6 (100 mg, crude) was obtained as yellow oil. LCMS (Method G): R^{t}=0.754 min, [M+H]⁺=1350.9. LCMS (Method G): R^{t}=0.754 min, [M+H]⁺=1350.9.

### Step 4: Synthesis of I-86

To a solution of I-86-6 (100 mg, 74.02 μmol, 1 eq) in dioxane (0.5 mL) was added HCl/dioxane (4 M, 0.5 mL, 27.02 eq). The mixture was stirred at 25° C. for 1 hr. The reaction mixture was concentrated under reduced pressure to give a residue. The residue was purified by prep-HPLC (column: C18 150×30 mm; mobile phase: [water (FA)-ACN]; gradient: 12%-42% B over 7 min). I-86 (23.17 mg, 19.36 μmol, 26.16% yield, 100% purity; FA) was obtained as a white solid. LCMS (Method E): R^{t}=0.418 min, [M+H]⁺=1151.0. SFC: R^{t}=0.907 min. ¹H NMR (400 MHz, METHANOL-d4) δ=9.34 (d, J=2.0 Hz, 1H), 8.60 (d, J=2.0 Hz, 1H), 8.27 (s, 1H), 8.17 (s, 1H), 7.59-7.49 (m, 2H), 7.44 (t, J=7.6 Hz, 1H), 7.38-7.27 (m, 5H), 7.17 (d, J=3.6 Hz, 1H), 6.65 (d, J=3.6 Hz, 1H), 4.99 (br t, J=6.4 Hz, 1H), 4.66 (br dd, J=4.2 Hz, 2H), 3.74-3.63 (m, 14H), 3.62-3.54 (m, 10H), 3.50 (br s, 2H), 3.44-3.38 (m, 2H), 2.95 (s, 3H), 2.85-2.70 (m, 4H), 2.62-2.52 (m, 3H), 2.51-86-2.36 (m, 6H), 2.36-2.25 (m, 2H), 2.22-2.10 (m, 3H), 2.09-1.92 (m, 2H), 1.9 I-86-1.77 (m, 2H), 1.73-1.55 (m, 1H), 1.30 (t, J=7.6 Hz, 3H).

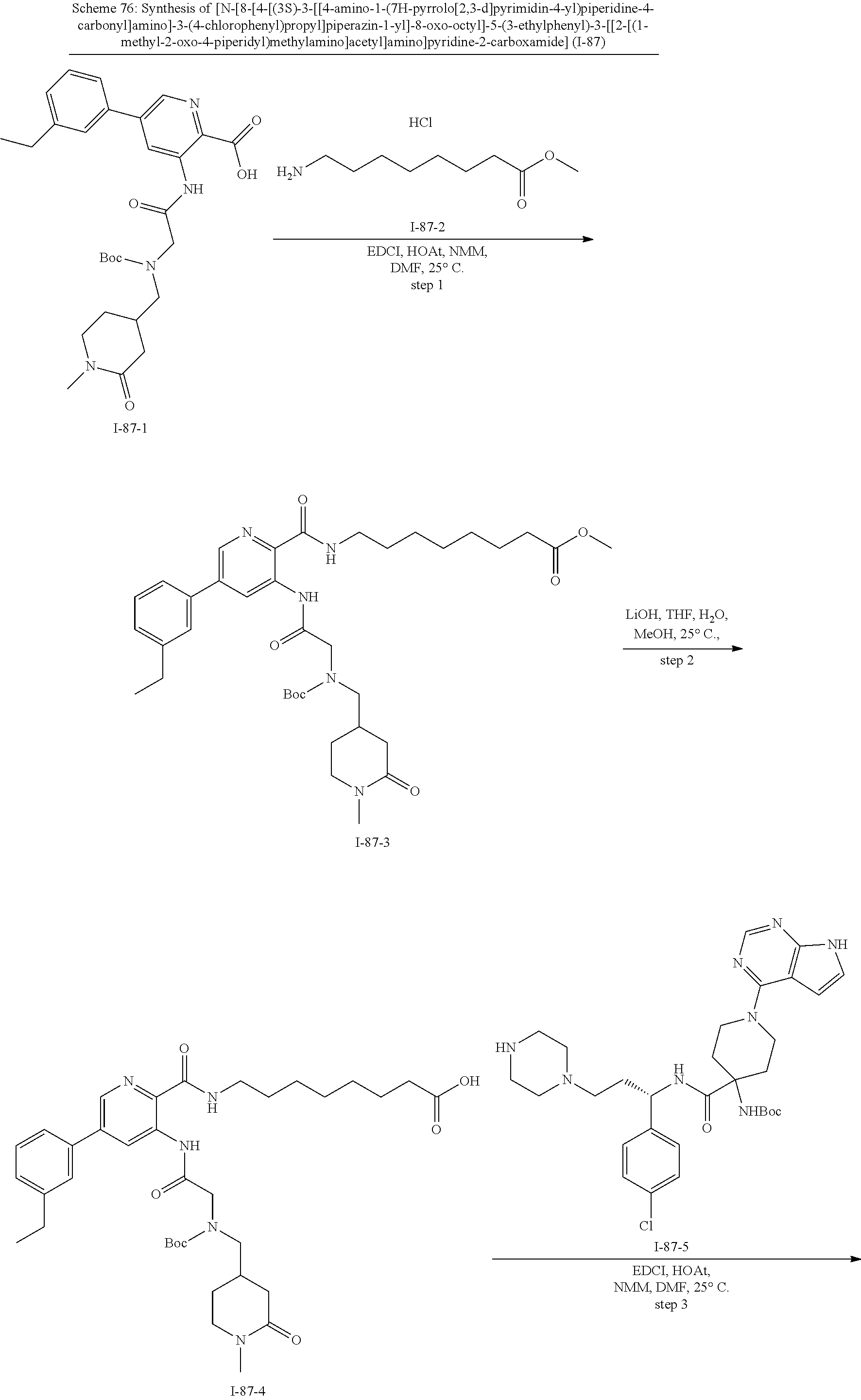 [SMILES: CCc1cccc(-c2cnc(C(=O)O)c(NC(=O)CN(CC3CCN(C)C(=O)C3)C(=O)OC(C)(C)C)c2)c1 ; COC(=O)CCCCCCCN ; Cl ; CCc1cccc(-c2cnc(C(=O)NCCCCCCCC(=O)OC)c(NC(=O)CN(CC3CCN(C)C(=O)C3)C(=O)OC(C)(C)C)c2)c1 ; CCc1cccc(-c2cnc(C(=O)NCCCCCCCC(=O)O)c(NC(=O)CN(CC3CCN(C)C(=O)C3)C(=O)OC(C)(C)C)c2)c1 ; CC(C)(C)OC(=O)NC1(C(=O)N[C@@H](CCN2CCNCC2)c2ccc(Cl)cc2)CCN(c2ncnc3[nH]ccc23)CC1]

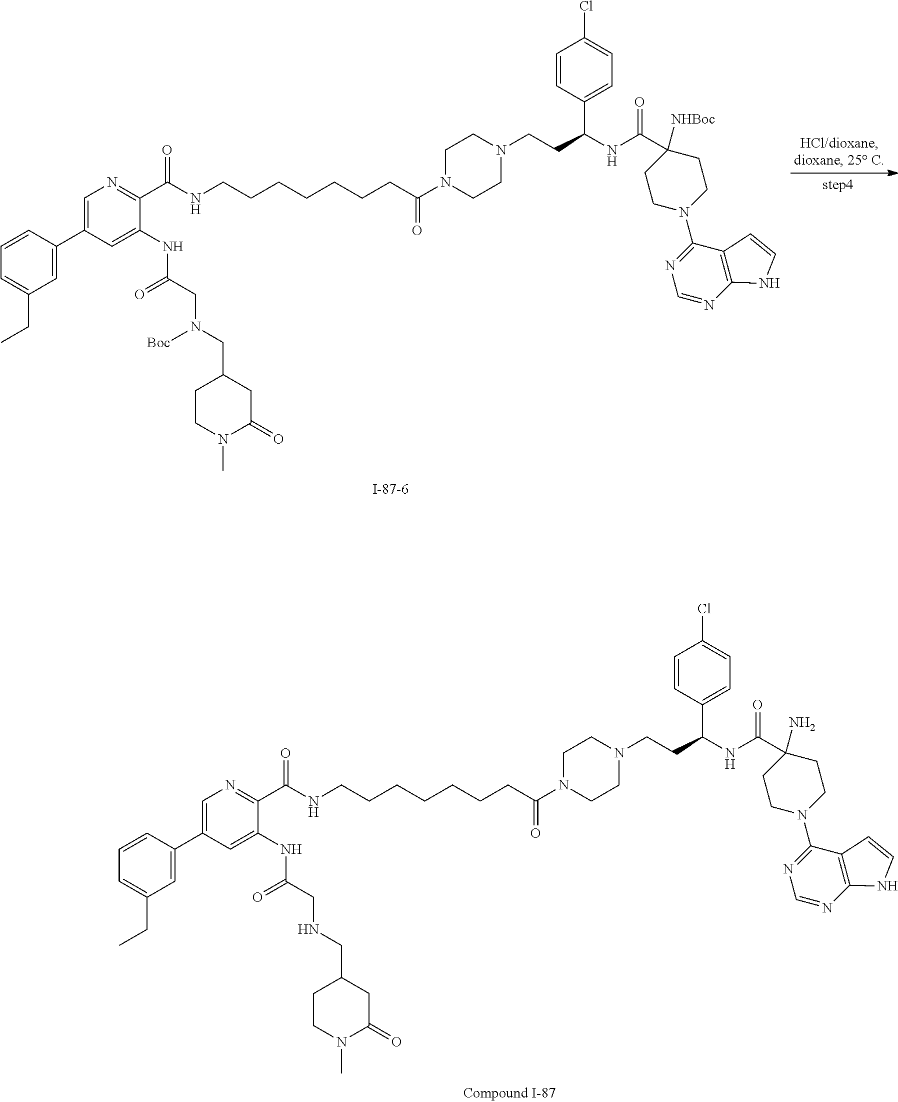 [SMILES: CCc1cccc(-c2cnc(C(=O)NCCCCCCCC(=O)N3CCN(CC[C@H](NC(=O)C4(NC(=O)OC(C)(C)C)CCN(c5ncnc6[nH]ccc56)CC4)c4ccc(Cl)cc4)CC3)c(NC(=O)CN(CC3CCN(C)C(=O)C3)C(=O)OC(C)(C)C)c2)c1 ; CCc1cccc(-c2cnc(C(=O)NCCCCCCCC(=O)N3CCN(CC[C@H](NC(=O)C4(N)CCN(c5ncnc6[nH]ccc56)CC4)c4ccc(Cl)cc4)CC3)c(NC(=O)CNCC3CCN(C)C(=O)C3)c2)c1]

### Step 1: Synthesis of I-87-3

To a solution of I-87-1 (80 mg, 152.50 μmol, 1 eq) and I-87-2 (48 mg, 228.74 μmol, 1.5 eq, HCl) in DMF (1 mL) was added HOBt (41 mg, 304.99 μmol, 2 eq), EDCI (58 mg, 304.99 μmol, 2 eq) and NMM (62 mg, 609.98 μmol, 67.06 μL, 4 eq). The mixture was stirred at 25° C. for 16 hr. Upon completion, the mixture was combined with another batch to work up and the reaction mixture was drop wised into water (10 mL) and extracted with ethyl acetate (5 mL×3). The combined organic layers were washed with brine (5 mL×2), dried over anhydrous sodium sulfate, filtered, and concentrated under reduced pressure to give a residue. The residue was purified by prep-HPLC (0.1% NH₃·H₂O) and the organic phase was concentrated under reduced pressure to remove acetonitrile, and the liquid was lyophilized to give I-87-3 (50 mg, 69.87 μmol, 45.82% yield, 95% purity) as yellow oil. LCMS (Method G): R^{t}=0.826 min, [M+H]⁺=680.4.

### Step 2: Synthesis of I-87-4

To a solution of I-87-3 (50 mg, 73.55 μmol, 1 eq) in THF (0.5 mL), H₂O (0.5 mL) and MeOH (0.5 mL) was added LiOH·H₂O (9 mg, 220.64 μmol, 3 eq). The mixture was stirred at 25° C. for 2 hr. Adjust PH to 6 by adding 1M HCl into the reaction solution, the reaction mixture was diluted into water (5 mL) and extracted with ethyl acetate (3 mL×3). The combined organic layers were washed with brine (5 ml×2), dried over anhydrous Na₂SO₄, filtered, and concentrated under reduced pressure to give I-87-4 (45 mg, 67.59 μmol, 91.90% yield) as yellow oil. LCMS (Method G): R^{t}=0.449 min, [M+H]⁺=666.4.

### Step 3: Synthesis of I-87-6

To a solution of I-87-4 (45 mg, 67.59 μmol, 1 eq) and I-87-5 (61 mg, 101.38 μmol, 1.5 eq) in DMF (1 mL) was added EDCI (39 mg, 202.76 μmol, 3 eq), HOAt (28 mg, 202.76 μmol, 3 eq) and NMM (34 mg, 337.93 μmol, 37.15 μL, 5 eq). The mixture was stirred at 25° C. for 16 hr. Upon completion, the reaction mixture was diluted into water (10 mL) and extracted with ethyl acetate (5 mL×3). The combined organic layers were washed with brine (5 mL×2), dried over anhydrous Na₂SO₄, filtered, and concentrated under reduced pressure to give I-87-6 (110 mg, crude) as yellow oil. LCMS (Method G): R^{t}=0.827 min, [M+H]⁺=623.

### Step 4: Synthesis of I-87

To a solution of I-87-6 (100 mg, 80.32 μmol, 1 eq) in dioxane (0.25 mL) was added HCl/dioxane (4 M, 5.00 mL, 248.99 eq). The mixture was stirred at 25° C. for 0.25 hr. Upon completion, the reaction mixture was concentrated under reduced pressure to give the crude product. The residue was dissolved in DMF (2 mL) and purified by prep-HPLC (column: C18 150×30 mm; mobile phase: [water (FA)-ACN]; gradient: 15%-45% B over 7 min) and the organic phase was concentrated under reduced pressure to remove acetonitrile, and the liquid was lyophilized to give I-87 (16.67 mg, 15.80 μmol, 19.67% yield, 99% purity) as a yellow solid. LCMS (Method E): R^{t}=0.458 min, [M+H]⁺=1044.6. SFC: R^{t}=5.294 min. 1H NMR (400 MHZ, METHANOL-d4) δ=9.34 (d, J=2.0 Hz, 1H), 8.57 (d, J=2.0 Hz, 1H), 8.44-8.38 (m, 1H), 8.14 (s, 1H), 7.55-7.47 (m, 2H), 7.45-7.38 (m, 1H), 7.35-7.26 (m, 5H), 7.15-7.10 (m, 1H), 6.62 (d, J=3.6 Hz, 1H), 5.00-4.94 (m, 1H), 4.62-4.51 (m, 2H), 3.65-3.48 (m, 8H), 3.46-3.35 (m, 4H), 2.93 (s, 3H), 2.74 (q, J=7.6 Hz, 2H), 2.67 (t, J=5.6 Hz, 2H), 2.58-2.50 (m, 1H), 2.47-2.28 (m, 8H), 2.27-2.09 (m, 5H), 2.07-1.90 (m, 2H), 1.74-1.52 (m, 7H), 1.40 (s, 6H), 1.29 (t, J=7.4 Hz, 3H)

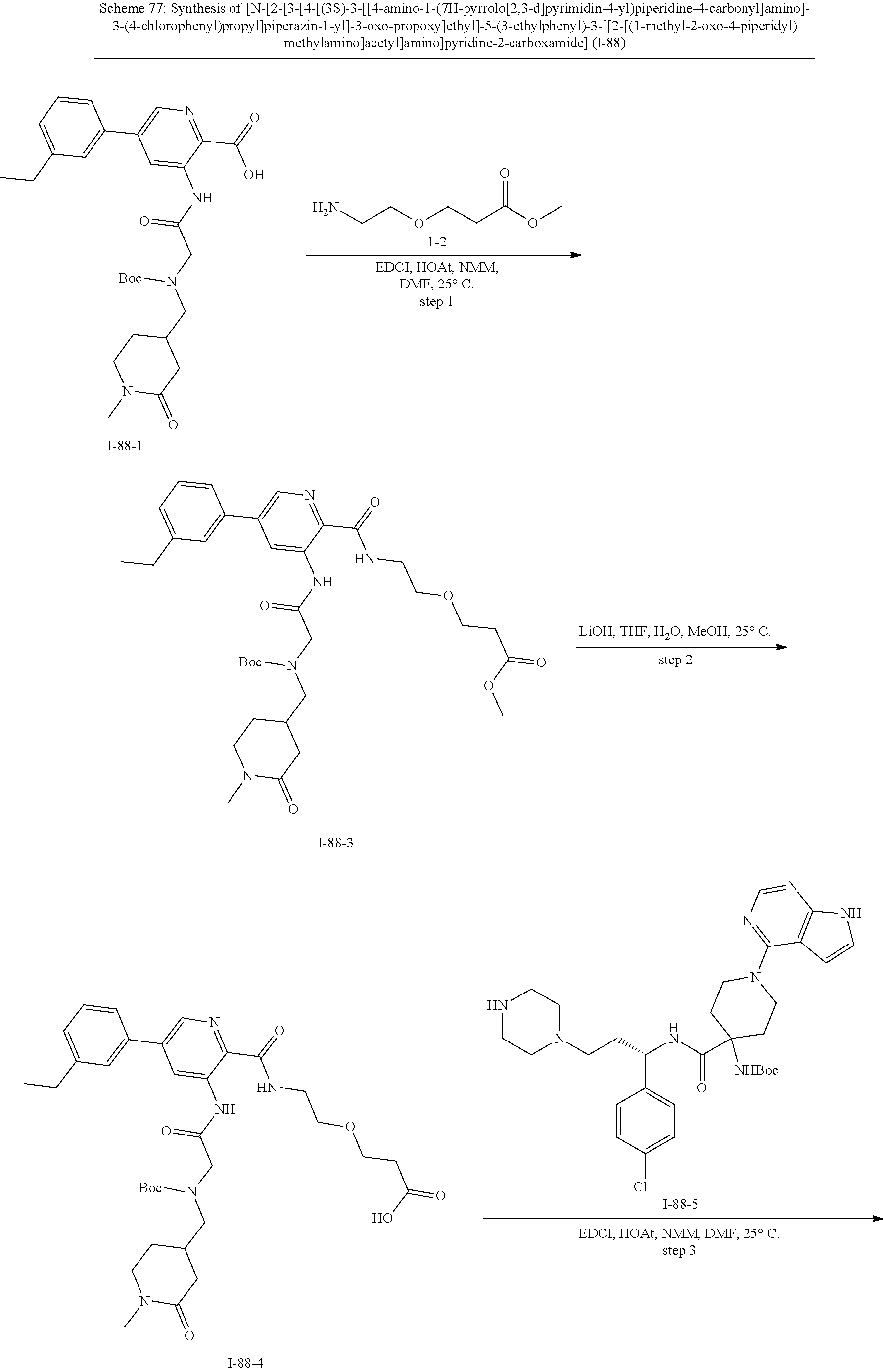

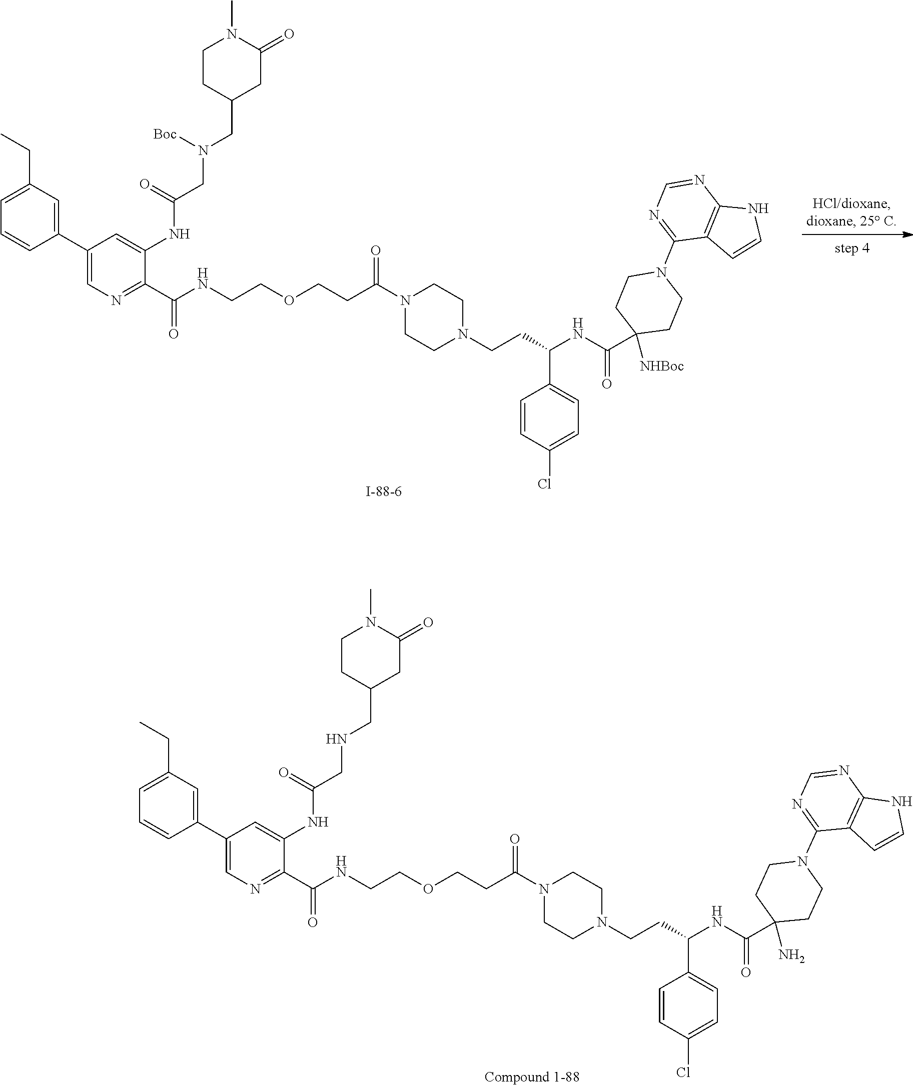 [SMILES: CCc1cccc(-c2cnc(C(=O)NCCOCCC(=O)N3CCN(CC[C@H](NC(=O)C4(NC(=O)OC(C)(C)C)CCN(c5ncnc6[nH]ccc56)CC4)c4ccc(Cl)cc4)CC3)c(NC(=O)CN(CC3CCN(C)C(=O)C3)C(=O)OC(C)(C)C)c2)c1 ; CCc1cccc(-c2cnc(C(=O)NCCOCCC(=O)N3CCN(CC[C@H](NC(=O)C4(N)CCN(c5ncnc6[nH]ccc56)CC4)c4ccc(Cl)cc4)CC3)c(NC(=O)CNCC3CCN(C)C(=O)C3)c2)c1]

### Step 1: Synthesis of I-88-3

To a solution of I-88-1 (80 mg, 152.50 μmol, 1 eq) and I-88-2 (34 mg, 228.74 μmol, 1.5 eq) in DMF (1 mL) was added HOBt (41 mg, 304.99 μmol, 2 eq), EDCI (58 mg, 304.99 μmol, 2 eq) and NMM (62 mg, 609.98 μmol, 67.06 μL, 4 eq). The mixture was stirred at 25° C. for 16 hr. Upon completion, the mixture was combined to work up and the reaction mixture was drop wised into water (10 mL) and extracted with ethyl acetate (5 mL×3). The combined organic layers were washed with brine (5 mL×2), dried over anhydrous Na₂SO₄, filtered, and concentrated under reduced pressure to give a residue. The residue was purified by prep-HPLC (0.1% NH₃·H₂O) and the organic phase was concentrated under reduced pressure to remove acetonitrile, and the liquid was lyophilized to give I-88-3 (50 mg, 73.42 μmol, 48.15% yield, 96% purity) as yellow oil. LCMS (Method G): R^{t}=0.718 min, [M+H]⁺=654.4.

### Step 2: Synthesis of I-88-4

To a solution of I-88-3 (50 mg, 76.48 μmol, 1 eq) in THF (0.5 mL), H2O (0.5 mL) and MeOH (0.5 mL) was added LiOH·H₂O (10 mg, 229.44 μmol, 3 eq). The mixture was stirred at 25° C. for 2 hrs. Upon completion, adjusted PH to 6 by adding 1M HCl into the reaction solution, the reaction mixture was diluted into water (5 mL) and extracted with ethyl acetate (3 mL×3). The combined organic layers were washed with brine (5 mL×2), dried over anhydrous Na₂SO₄, filtered, and concentrated under reduced pressure to give I-88-4 (60 mg, crude) as yellow oil. LCMS (Method G): R^{t}=0.421 min, [M+H]⁺=640.4.

### Step 3: Synthesis of I-88-6

To a solution of I-88-4 (60 mg, 93.79 μmol, 1 eq) and I-88-5 (84 mg, 140.68 μmol, 1.5 eq) in DMF (1 mL) was added EDCI (54 mg, 281.37 μmol, 3 eq), HOAt (38 mg, 281.37 μmol, 3 eq) and NMM (47 mg, 468.94 μmol, 51.56 μL, 5 eq). The mixture was stirred at 25° C. for 16 hrs. Upon completion, the reaction mixture was diluted into water (10 mL) and extracted with ethyl acetate (5 mL×3). The combined organic layers were washed with brine (5 mL×2), dried over anhydrous Na₂SO₄, filtered, and concentrated under reduced pressure to give I-88-6 (140 mg, crude) as yellow oil. LCMS (Method G): R^{t}=0.753 min, [M+H]⁺=597.4.

### Step 4: Synthesis of I-88

To a solution of I-88-6 (100 mg, 82.04 μmol, 1 eq) in dioxane (0.25 mL) was added HCl/dioxane (4 M, 5.00 mL, 243.77 eq). The mixture was stirred at 25° C. for 0.25 hr. Upon completion, the reaction mixture was concentrated under reduced pressure to give the crude product. The residue was dissolved in DMF (2 mL) and purified by prep-HPLC (column: C18150×30 mm; mobile phase: [water (FA)-ACN]; gradient: 10%-40% B over 7 min) and the organic phase was concentrated under reduced pressure to remove acetonitrile, and the liquid was lyophilized to give I-88 (13.11 mg, 12.87 μmol, 15.69% yield, 100% purity) as a yellow solid. LCMS (Method E): R^{t}=0.423 min, [M+H]⁺=1018.6. SFC: R^{t}=8.864 min. 1H NMR (400 MHZ, METHANOL-d4) δ=9.33 (d, J=2.0 Hz, 1H), 8.57 (d, J=2 Hz, 1H), 8.45-8.30 (m, 1H), 8.14 (s, 1H), 7.52-7.44 (m, 2H), 7.41 (t, J=7.4 Hz, 1H), 7.33-7.20 (m, 5H), 7.13 (d, J=3.6 Hz, 1H), 6.61 (d, J=3.6 Hz, 1H), 4.93-4.88 (m, 1H), 4.63-4.51 (m, 2H), 3.78 (t, J=6.0 Hz, 2H), 3.70-3.64 (m, 2H), 3.64-3.48 (m, 10H), 3.42-3.34 (m, 2H), 2.92 (s, 3H), 2.77-2.53 (m, 7H), 2.33 (s, 4H), 2.26-2.07 (m, 7H), 1.98-1.79 (m, 2H), 1.74-1.55 (m, 3H), 1.31-1.22 (m, 3H).

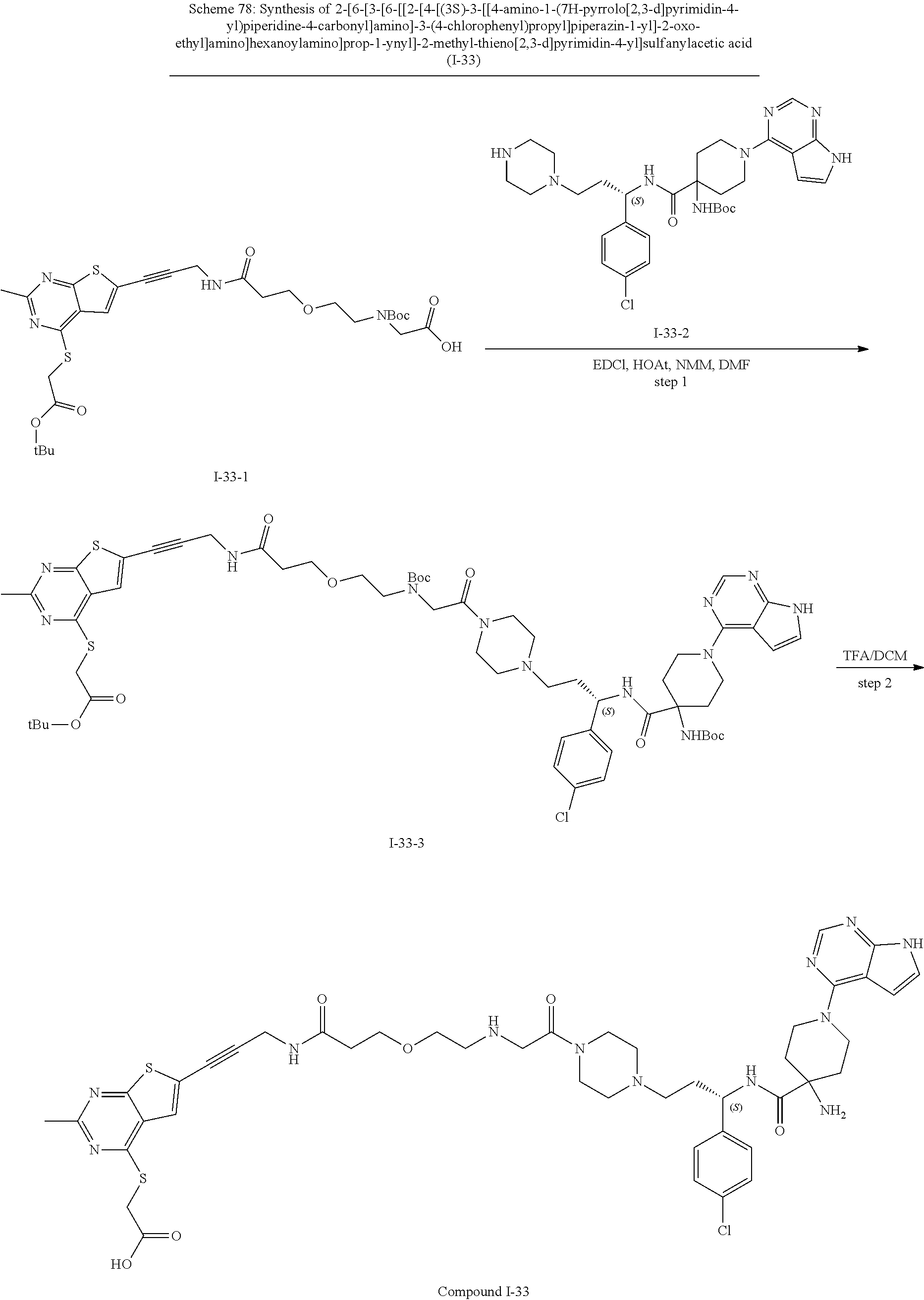

### Step 1: Synthesis of I-33-3

To a solution of I-33-1 (10 mg, 16.11 μmol, 1 eq) and I-33-2 (12.02 mg, 16.11 μmol, 1 eq) in DMF (1 mL) was added EDCI (6.18 mg, 32.22 μmol, 2 eq) NMM (8.15 mg, 80.54 μmol, 8.86 μL, 5 eq) and HOAt (1.10 mg, 8.05 μmol, 0.5 eq). The mixture was stirred at 25° C. for 4 hr. The mixture was diluted with H₂O (5 mL) and extracted with EA (5 mL*3). The combined organic layers were dried over anhydrous Na2SO4, filtered and concentrated under reduced pressure to give I-33-3 (15 mg, 12.50 μmol, 77.60% yield) as a white solid, which was used for next step without further purification. LCMS (Method E): R^{t}=0.563 min, [M+H]⁺=1199.8.

### Step 2: Synthesis of I-33

To a solution of I-33-3 (10 mg, 8.33 μmol, 1 eq) in DCM (0.3 mL) was added TFA (0.1 mL). The mixture was stirred at 25° C. for 1 hr. The reaction mixture was concentrated under the pressure to give residue. The residue was diluted with 0.5 mL MeOH. The residue was purified by prep-HPLC (column: Waters Xbridge 150*25 mm*5 um; mobile phase: [water (NH4HCO3)-ACN]; gradient: 12%-42% B over 9 min). I-33 (2.31 mg, 2.45 μmol, 29.38% yield, 100% purity) was obtained as an off-white solid. LCMS (Method E): R^{t}=0.402 min, [M+H]⁺=943.7. SFC: R^{t}=1.156 min. ¹H NMR (400 MHZ, METHANOL-d4) δ=8.12 (s, 1H), 7.42 (s, 1H), 7.32 (s, 4H), 7.12 (d, J=3.6 Hz, 1H), 6.61 (d, J=3.6 Hz, 1H), 5.00-4.96 (m, 1H), 4.54-4.44 (m, 2H), 4.24 (s, 2H), 4.07 (s, 2H), 3.97 (s, 2H), 3.70-3.57 (m, 4H), 3.39 (s, 2H), 3.03-2.95 (m, 2H), 2.65 (s, 3H), 2.57-1.85 (m, 14H), 1.81-1.69 (m, 4H), 1.68-1.60 (m, 2H), 1.49-1.40 (m, 2H).

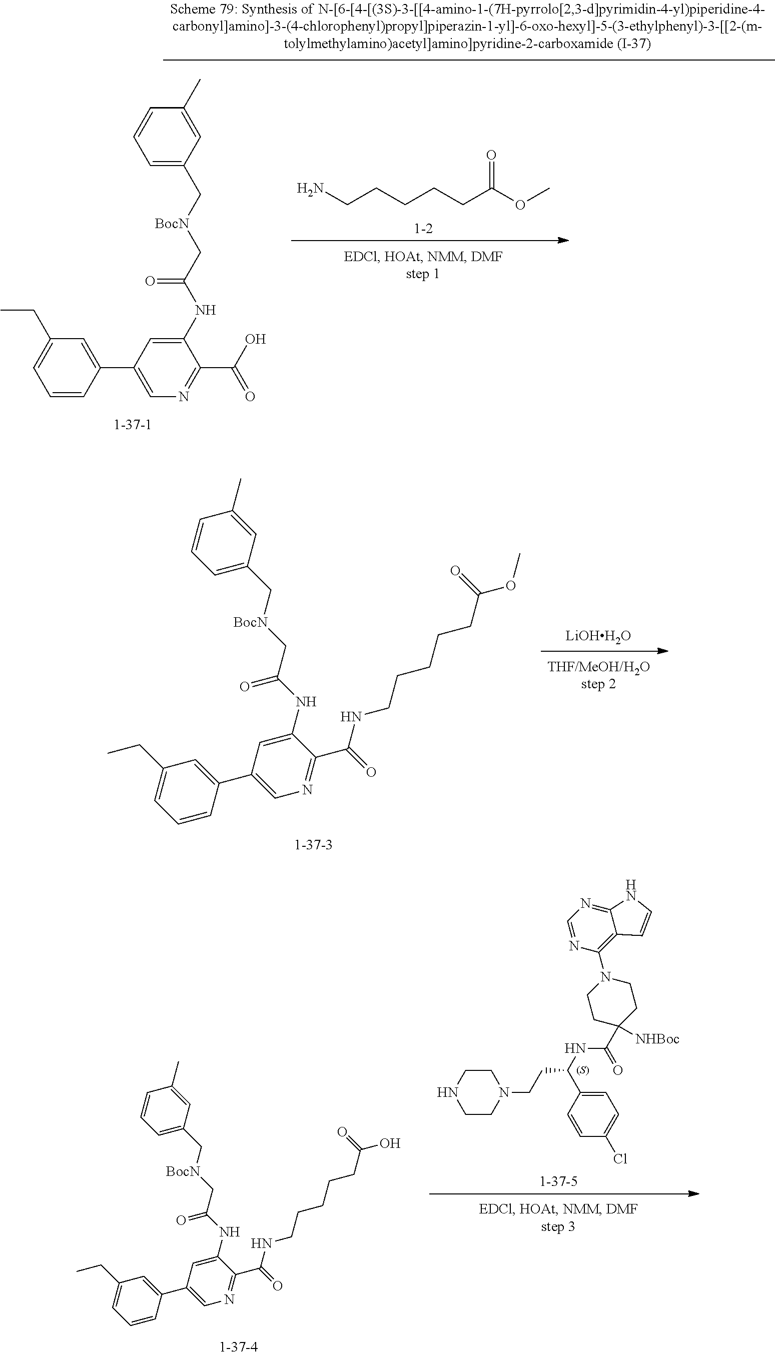

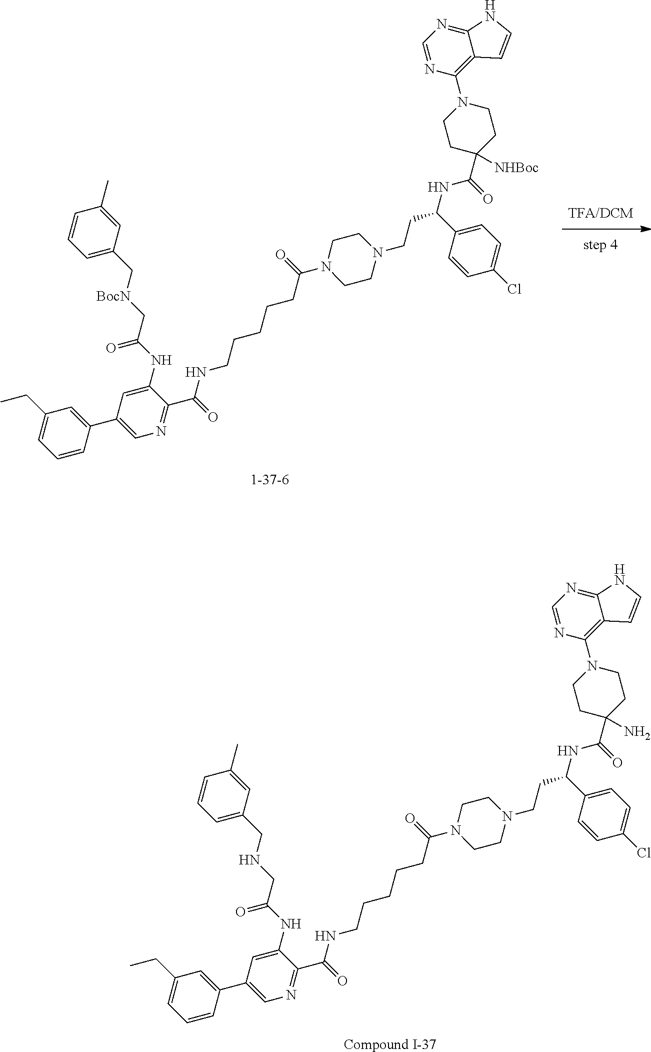 [SMILES: CCc1cccc(-c2cnc(C(=O)NCCCCCC(=O)N3CCN(CC[C@H](NC(=O)C4(NC(=O)OC(C)(C)C)CCN(c5ncnc6[nH]ccc56)CC4)c4ccc(Cl)cc4)CC3)c(NC(=O)CN(Cc3cccc(C)c3)C(=O)OC(C)(C)C)c2)c1 ; "1-37-6" ; CCc1cccc(-c2cnc(C(=O)NCCCCCC(=O)N3CCN(CC[C@H](NC(=O)C4(N)CCN(c5ncnc6[nH]ccc56)CC4)c4ccc(Cl)cc4)CC3)c(NC(=O)CNCc3cccc(C)c3)c2)c1 ; "Compound I-37"]

Step 1: Synthesis of I-37-3

To a solution of I-37-1 (150 mg, 297.86 μmol, 1 eq), I-37-2 (81.16 mg, 446.79 μmol, 1.5 eq, HCl) in DMF (1.5 mL), then the NMM (150.64 mg, 1.49 mmol, 163.74 μL, 5 eq), EDCI (285.50 mg, 1.49 mmol, 5 eq) and HOAt (81.08 mg, 595.72 μmol, 83.33 μL, 2 eq) was added in. The mixture was stirred at 25° C. for 1 h. The reaction mixture was washed with water (2 mL) and extracted with DCM (2 mL*3), the combined organic phase was dried by Na₂SO₄, concentrated under reduced pressure to give a residue. There residue was purified by flash silica gel chromatography (ISCO®; 12 g SepaFlash® Silica Flash Column, Eluent of 0˜50% Ethyl acetate/Petroleum ether (@35 mL/min) and the eluent was concentrated to give product. I-37-3 (285 mg, crude) was obtained as a yellow solid. LCMS (Method D): R^{t}=0.616 min, [M+H]⁺=631.5.

### Step 2: Synthesis of I-37-4

To a solution of I-37-3 (235 mg, 372.56 μmol, 1 eq) in THF (0.2 mL) and MeOH (0.2 mL) was added LiOH·H₂O (46.90 mg, 1.12 mmol, 3 eq) in H₂O (0.2 mL) at 25° C. The mixture was stirred at 25° C. for 1 hr. The pH was adjusted to 7 with 1M HCl, the reaction mixture was washed with water (2 mL) and extracted with DCM (2 mL*3), and the combined organic phase was dried by Na₂SO₄, concentrated under reduced pressure to give a residue. The crude product was used into the next step without further purification. I-37-4 (280 mg, crude) was obtained as yellow oil. LCMS (Method D): R^{t}=0.535 min, [M+H]⁺=617.4. ¹H NMR (400 MHZ, METHANOL-d4) δ=9.11 (s, 1H), 8.43 (s, 1H), 7.37 (d, J=13.2 Hz, 2H), 7.29 (t, J=7.6 Hz, 1H), 7.18 (d, J=7.2 Hz, 1H), 7.09-7.04 (m, 1H), 7.00 (d, J=11.6 Hz, 2H), 6.92 (d, J=7.2 Hz, 1H), 4.50 (s, 2H), 3.88-3.75 (m, 4H), 3.31 (s, 2H), 3.20 (s, 2H), 2.67-2.53 (m, 3H), 1.55 (d, J=6.0 Hz, 4H), 1.42 (s, 2H), 1.33 (s, 9H), 1.17 (s, 3H).

### Step 3: Synthesis of I-37-5

To a solution of I-37-4 (30 mg, 48.64 μmol, 1 eq), I-37-5 (29.05 mg, 48.64 μmol, 1 eq) in DMF (0.5 mL) was added NMM (24.60 mg, 243.21 μmol, 26.74μ, 5 eq), EDCI (46.62 mg, 243.21 μmol, 5 eq) and HOAt (13.24 mg, 97.28 μmol, 13.61 μL, 2 eq). The mixture was stirred at 25° C. for 1 h. The reaction was washed with water (1 mL) and extracted with DCM (1 mL*3), the combined organic phase was dried by Na₂SO₄, concentrated under reduced pressure to give a residue. The residue was purified by flash silica gel chromatography (ISCO®; 4 g SepaFlash® Silica Flash Column, Eluent of 0˜50% Ethyl acetate/Petroleum ether (20 mL/min) and the eluent was concentrated to give product. I-37-6 (50 mg, 41.81 μmol, 85.95% yield, 100% purity) was obtained as colorless oil. LCMS (Method D): R^{t}=0.504 min, [M+H]⁺=1195.6. ¹H NMR (400 MHZ, METHANOL-d4) δ=9.24 (s, 1H), 8.58 (s, 1H), 8.28 (s, 1H), 7.53-7.48 (m, 2H), 7.45-7.39 (m, 3H), 7.38-7.31 (m, 4H), 7.19-7.08 (m, 3H), 7.04 (d, J=7.2 Hz, 1H), 6.94 (d, J=2.0 Hz, 1H), 5.07 (d, J=4.8 Hz, 1H), 4.61 (s, 3H), 4.42-4.29 (m, 2H), 4.06 (s, 1H), 3.96 (s, 1H), 3.87-3.80 (m, 2H), 3.72-3.52 (m, 3H), 3.46 (s, 2H), 3.17-3.04 (m, 2H), 2.74 (q, J=7.2 Hz, 2H), 2.48 (d, J=0.8 Hz, 2H), 2.35 (s, 3H), 2.27 (s, 4H), 2.20 (s, 2H), 1.75-1.63 (m, 5H), 1.60-1.52 (m, 5H), 1.50-1.50 (m, 1H), 1.51-1.43 (m, 8H), 1.42 (s, 9H), 1.29 (t, J=7.6 Hz, 4H), 1.00-0.94 (m, 1H).

### Step 4: Synthesis of I-37

To a solution of I-37-6 (50 mg, 24.62 μmol, 1 eq) in DCM (0.3 mL) added TFA (767.50 mg, 6.73 mmol, 500.00 μL, 273.40 eq). The mixture was stirred at 25° C. for 1 hr. The mixture was concentrated under reduced pressure to give a residue. The crude was used next step and added ACN and H₂O to lyophilized to get product and no further purification. I-37 (27 mg, 24.33 μmol, 98.83% yield, 100% purity, TFA) was obtained as a white solid. LCMS (Method D): R^{t}=0.371 min, [M+H]⁺=995.5. SFC: R^{t}=0.981 min. ¹H NMR (400 MHZ, METHANOL-d4) δ=9.22 (s, 1H), 8.63 (s, 1H), 8.33 (s, 1H), 7.51 (d, J=11.6 Hz, 2H), 7.44 (t, J=7.6 Hz, 1H), 7.40-7.28 (m, 9H), 6.85 (d, J=3.2 Hz, 1H), 5.04-4.99 (m, 1H), 4.67-4.60 (m, 2H), 4.29 (s, 2H), 4.15 (s, 2H), 3.99-3.64 (m, 5H), 3.43 (t, J=6.4 Hz, 2H), 3.26-3.03 (m, 4H), 2.79-2.72 (m, 2H), 2.64-2.52 (m, 2H), 2.50-2.20 (m, 6H), 2.13 (d, J=14.4 Hz, 1H), 2.00 (d, J=13.6 Hz, 1H), 1.72-1.63 (m, 4H), 1.49-1.41 (m, 2H), 1.38-1.24 (m, 5H), ¹⁹F NMR (377 MHz, METHANOL-d4) δ=−77.045.

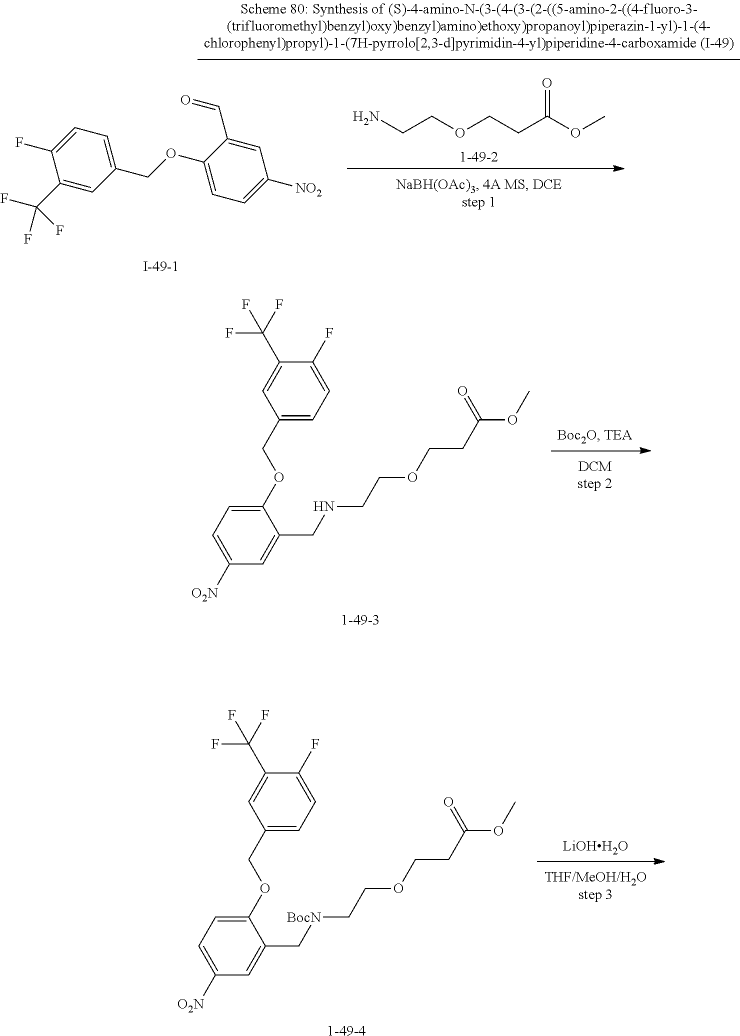

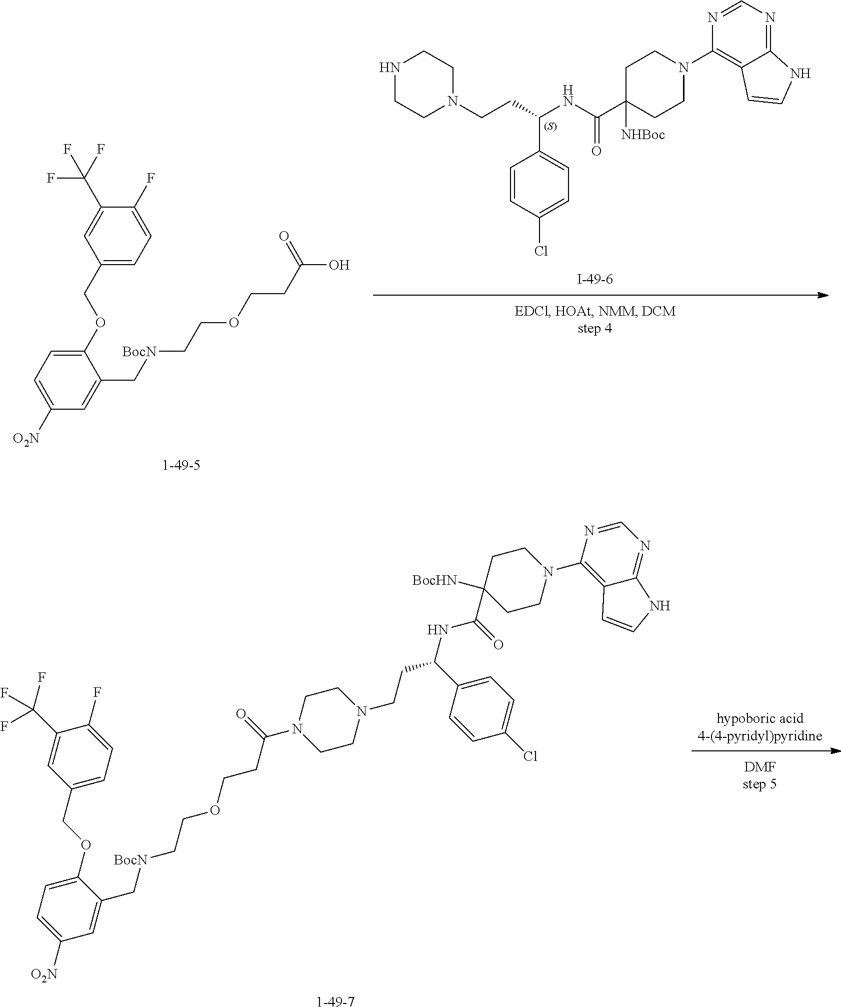

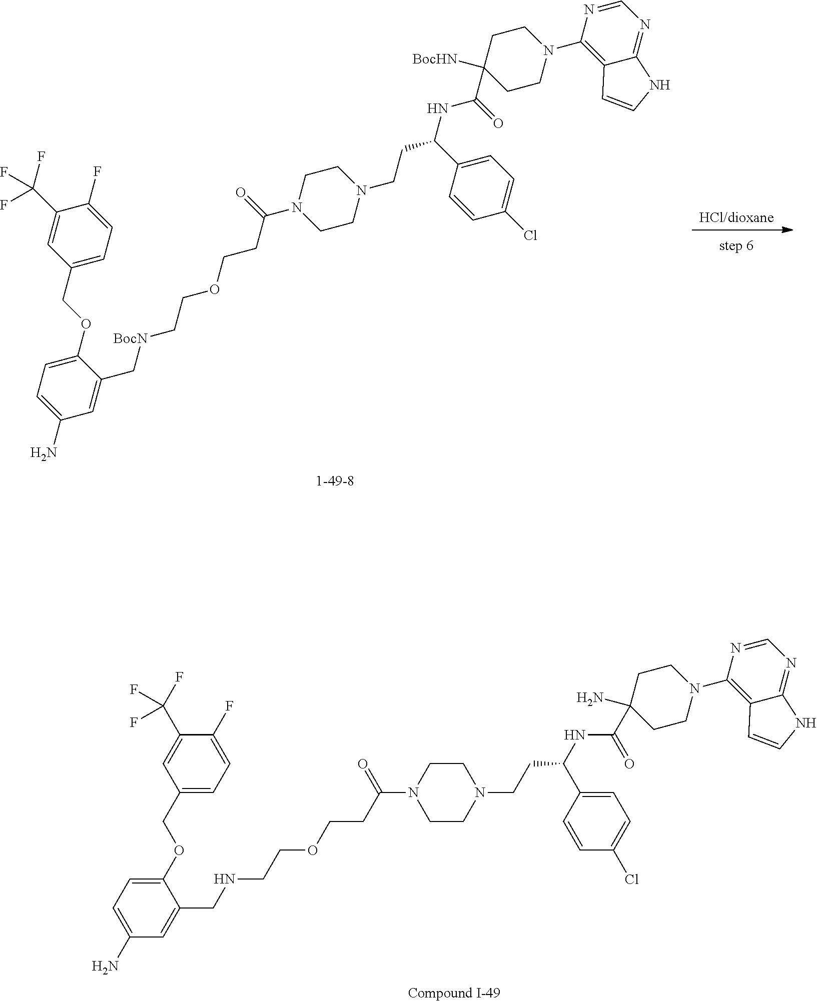 [SMILES: CC(C)(C)OC(=O)NC1(C(=O)N[C@@H](CCN2CCN(C(=O)CCOCCN(Cc3cc(N)ccc3OCc3ccc(F)c(C(F)(F)F)c3)C(=O)OC(C)(C)C)CC2)c2ccc(Cl)cc2)CCN(c2ncnc3[nH]ccc23)CC1 ; "HCl/dioxane" ; "1-49-8" ; Nc1ccc(OCc2ccc(F)c(C(F)(F)F)c2)c(CNCCOCCC(=O)N2CCN(CC[C@H](NC(=O)C3(N)CCN(c4ncnc5[nH]ccc45)CC3)c3ccc(Cl)cc3)CC2)c1 ; "Compound I-49"]

### Step 1: Synthesis of I-49-3

A solution of I-49-1 (200 mg, 582.70 μmol, 1 eq) and 4 A MS (50 mg) in DCE (3 mL) was stirred at 25° C. for 0.25 h under N° atmosphere. Then I-49-2 (107.00 mg, 582.70 μmol, 1 eq, HCl) was added to the mixture, and the mixture was stirred at 25° C. for 0.75 h, then NaBH(OAc)₃ (308.75 mg, 1.46 mmol, 2.5 eq) was added to the mixture and the mixture was stirred at 25° C. for 1 hr. The reaction mixture was quenched with water (5 mL) and extracted with DCM (5 mL*3). The organic layers were dried over anhydrous Na₂SO₄, filtered and the filtrate was concentrated under vacuum. The residue was purified by flash silica gel chromatography (ISCO®; 12 g SepaFlash® Silica Flash Column, Eluent of 40˜100% Ethyl acetate/Petroleum ether gradient @ 20 mL/min). I-49-3 (130 mg, 274.03 μmol, 47.03% yield, 100% purity) was obtained as a white solid, LCMS (Method E): R^{t}=0.484 min, [M+H]⁺=475.2,

### Step 2: Synthesis of I-49-4

To a solution of I-49-3 (130 mg, 274.03 μmol, 1 eq) in DCM (2 mL) was added Boc20 (179.42 mg, 822.09 μmol, 188.86 μL, 3 eq) and TEA (55.46 mg, 548.06 μmol, 76.28 μL, 2 eq). The mixture was stirred at 25° C. for 1 hr. The reaction mixture was quenched with water (5 mL). The mixture was extracted with EA (5 mL*3) and dried over anhydrous Na₂SO₄. The mixture filtered and the filtrate was concentrated under vacuum. The residue was purified by flash silica gel chromatography (ISCO®; 20 g SepaFlash® Silica Flash Column, Eluent of 0˜30% Ethyl acetate/Petroleum ether gradient @ 20 mL/min). I-49-4 (130 mg, 219.49 μmol, 80.10% yield, 97% purity) was obtained as a white solid. LCMS (Method E): R^{t}=0.651 min, [M+H-Boc]⁺=475.3.

### Step 3: Synthesis of I-49-5

To a solution of I-49-4 (100 mg, 174.06 μmol, 1 eq) in THF (1 mL) was added LiOH·H₂O (14.61 mg, 348.12 μmol, 2 eq) in H2O (1 mL) and MeOH (1 mL). The mixture was stirred at 25° C. for 1 hr. The reaction mixture was quenched with water (5 mL) and acidify with HCl (0.5 M, 1 mL). The mixture was extracted with EA (5 mL*3) and the organic layers were dried over anhydrous Na₂SO₄. The mixture filtered and the filtrate was concentrated under vacuum. The residue was used to next step without further purification. I-49-5 (100 mg, crude) was obtained as a white solid, LCMS (Method E): R^{t}=0.602 min, [M+H-Boc]⁺=461.1.

### Step 4: Synthesis of I-49-7

To a solution of I-49-5 (94 mg, 145.91 μmol, 1 eq) and I-49-6 (87.13 mg, 145.91 μmol, 1 eq) in DCM (1 mL) was added EDCI (55.94 mg, 291.82 μmol, 2 eq), NMM (73.79 mg, 729.54 μmol, 80.21 μL, 5 eq) and HOAt (9.93 mg, 72.95 μmol, 0.5 eq). The mixture was stirred at 25° C. for 1 hr. The reaction mixture was quenched with water (5 mL) and then extracted with EA (5 mL*3). The organic layers were dried over anhydrous Na₂SO₄, filtered and the filtrate was concentrated under vacuum. The residue was purified by flash silica gel chromatography (ISCO®; 12 g SepaFlash® Silica Flash Column, Eluent of 0˜30% Ethyl acetate/Petroleum ether gradient @ 20 mL/min). I-49-7 (130 mg, 114.07 μmol, 78.18% yield) was obtained as a white solid. LCMS (Method E): R^{t}=0.560 min, [M+H]⁺=1140.0.

### Step 5: Synthesis of I-49-8

To a solution of I-49-7 (50 mg, 43.87 μmol, 1 eq) in DMF (1 mL) was added hypoboric acid (23.60 mg, 263.24 μmol, 6 eq) and 4-(4-pyridyl)pyridine (3.43 mg, 21.94 μmol, 0.5 eq) at 0° C., then it was warmed to 25° C. The mixture was stirred at 25° C. for 1 hr. The reaction mixture was quenched with water (5 mL) and then extracted with EA (5 mL*3). The organic layers were dried over anhydrous Na₂SO₄, filtered and the filtrate was concentrated under vacuum. The residue was used to next step without further purification. I-49-8 (50 mg, crude) was obtained as a white solid. LCMS (Method E): R^{t}=0.488 min, [M+H]⁺=1109.8.

### Step 6: Synthesis of I-49

A solution of I-49-8 (50 mg, 45.06 μmol, 1 eq) in HCl/dioxane (1 mL) was stirred at 25° C. for 1 hr. The reaction mixture was concentrated under the pressure to give crude product, and diluted by MeOH (1 mL). The residue was purified by prep-HPLC (column: Phenomenex luna C18 150*25 mm*10 um; mobile phase: [water (FA)-ACN]; gradient: 1%-30% B over 10 min). I-49 (11.94 mg, 12.50 μmol, 27.73% yield, 100% purity, FA) was obtained as a brown solid. LCMS (Method E): R^{t}=0.397 min, [M+H]⁺=909.6. SFC: R^{t}=1.068 min. ¹H NMR (400 MHZ, CHLOROFORM-d) δ=8.96-8.80 (m, 1H), 7.97 (s, 1H), 7.66-7.57 (m, 2H), 7.32-7.29 (m, 2H), 7.24-7.19 (m, 1H), 7.15-7.08 (m, 3H), 6.87 (d, J=2.8 Hz, 1H), 6.70-6.64 (m, 1H), 6.63-6.57 (m, 1H), 6.46 (d, J=3.6 Hz, 1H), 5.07-5.01 (m, 1H), 4.93 (s, 2H), 4.87-4.69 (m, 2H), 4.16 (s, 2H), 3.88-3.80 (m, 2H), 3.75-3.65 (m, 4H), 3.61-3.26 (m, 4H), 3.15 (s, 2H), 2.94 (d, J=1.6 Hz, 2H), 2.47-2.25 (m, 6H), 2.25-2.14 (m, 4H), 2.13-2.05 (m, 2H), 2.00-1.68 (m, 4H), ¹⁹F NMR 377 MHz, CHLOROFORM-d) δ=−61.324, −115.176.

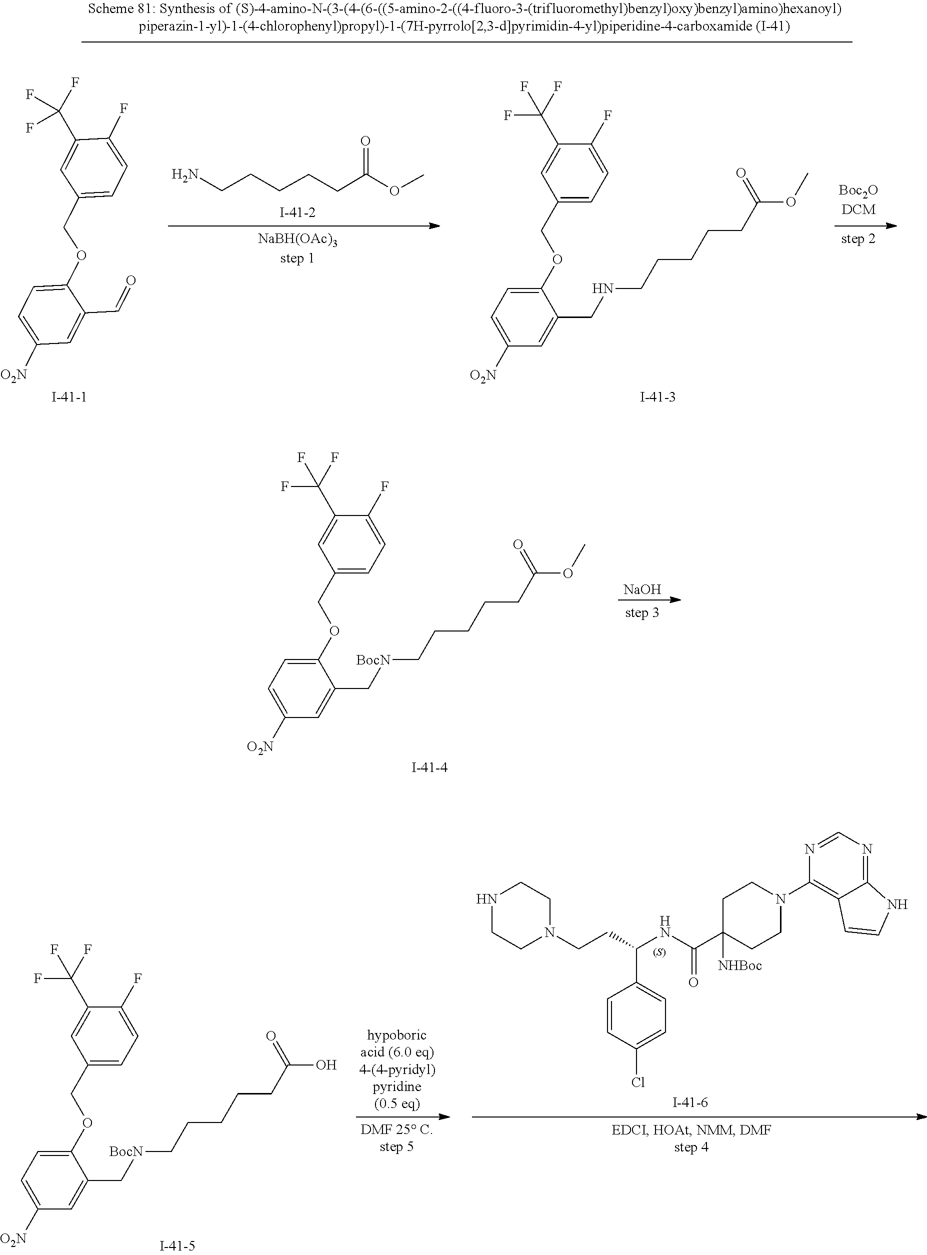

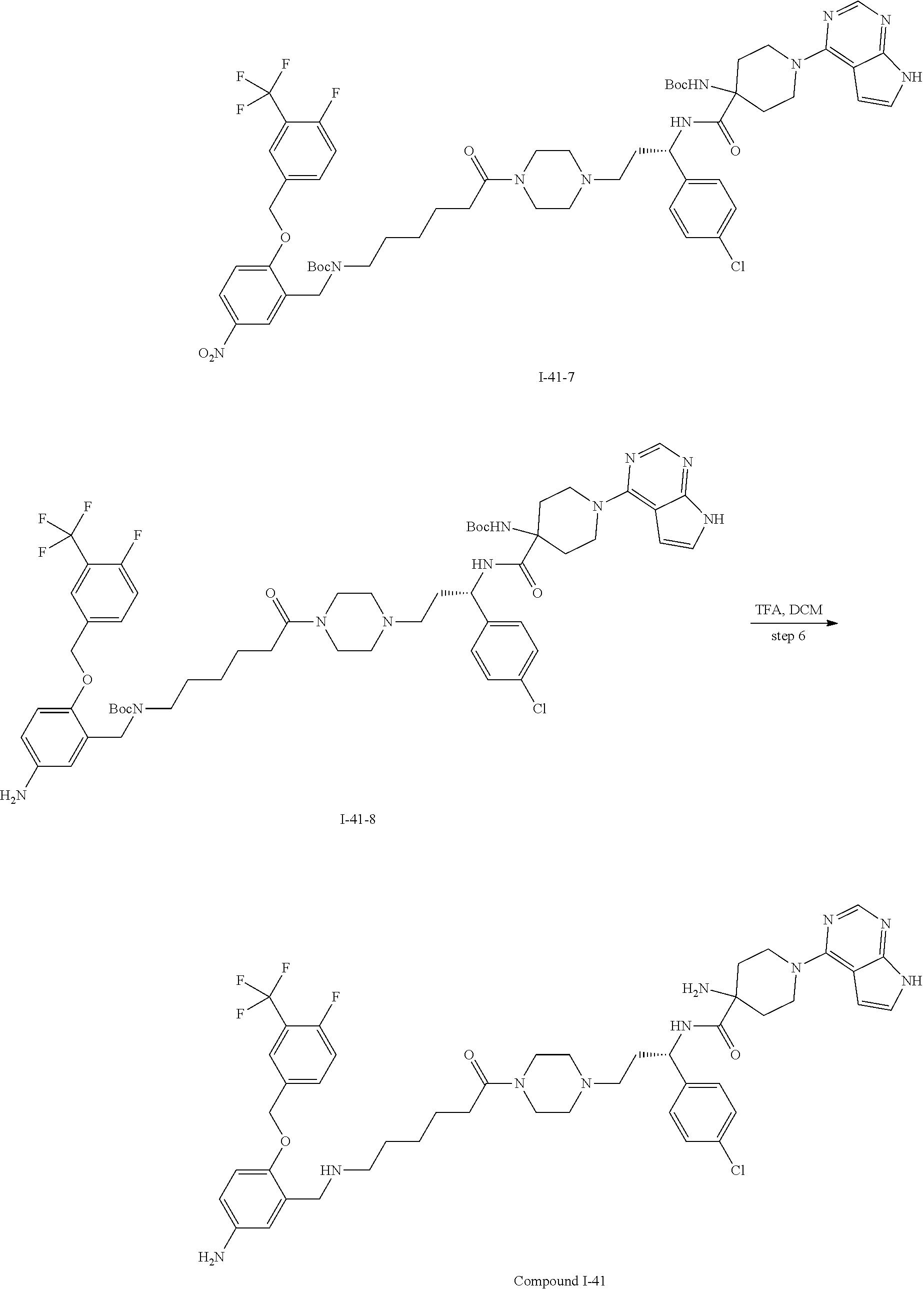 [SMILES: CC(C)(C)OC(=O)NC1(C(=O)N[C@@H](CCN2CCN(C(=O)CCCCCN(Cc3cc([N+](=O)[O-])ccc3OCc3ccc(F)c(C(F)(F)F)c3)C(=O)OC(C)(C)C)CC2)c2ccc(Cl)cc2)CCN(c2ncnc3[nH]ccc23)CC1 ; CC(C)(C)OC(=O)NC1(C(=O)N[C@@H](CCN2CCN(C(=O)CCCCCN(Cc3cc(N)ccc3OCc3ccc(F)c(C(F)(F)F)c3)C(=O)OC(C)(C)C)CC2)c2ccc(Cl)cc2)CCN(c2ncnc3[nH]ccc23)CC1 ; Nc1ccc(OCc2ccc(F)c(C(F)(F)F)c2)c(CNCCCCCC(=O)N2CCN(CC[C@H](NC(=O)C3(N)CCN(c4ncnc5[nH]ccc45)CC3)c3ccc(Cl)cc3)CC2)c1]

### Step 1: Synthesis of I-41-3

To a solution of I-41-2 (105.85 mg, 582.70 μmol, 1 eq. HCl) and 4 A MS (50 mg) in DCE (3 mL) stirred at 25° C. for 0.25 h under N₂ atmosphere. Then I-41-1 (200 mg, 582.70 μmol, 1 eq) was added to the mixture, and the mixture was stirred at 25° C. for 0.75 h, then NaBH(OAc)₃ (308.75 mg, 1.46 mmol, 2.5 eq) was added to the mixture and the mixture was stirred at 25° C. for 1 hr. The mixture was poured into water 10 mL and extracted with EA (10 mL*3). The combined organic layers were concentrated to give a residue. The residue was purified by flash silica gel chromatography (ISCO®; 4 g SepaFlash® Silica Flash Column, Eluent of 0˜100% Ethyl acetate/Petroleum ether gradient @ 18 mL/min), and the eluent was concentrated to give I-41-3 (140 mg, 290.41 μmol, 49.84% yield, 98% purity) as a yellow solid. LCMS (Method E): R^{t}=0.483 min, [M+H]⁺=473.2.

### Step 2: Synthesis of I-41-4

To a solution of I-41-3 (140 mg, 296.34 μmol, 1 eq) in DCM (1 mL) was added Boc₂O (194.03 mg, 889.02 μmol, 204.24 μL, 3 eq) and TEA (59.97 mg, 592.68 μmol, 82.49 μL, 2 eq). The mixture was stirred at 25° C. for 2 hr. The reaction mixture was concentrated to give the crude product. The residue was purified by flash silica gel chromatography (ISCO®; 4 g SepaFlash® Silica Flash Column, Eluent of 0˜100% Ethyl acetate/Petroleum ether gradient @ 18 mL/min), and the eluent was concentrated to give the product. I-41-4 (160 mg, 193.41 μmol, 65.27% yield, 69.21% purity) was obtained as a yellow solid. LCMS (Method E): R^{t}=0.688 min, [M-Boc+H]⁺=473.2.

### Step 3: Synthesis of I-41-5

To a solution of I-41-4 (150 mg, 261.99 μmol, 1 eq) in THF (1.5 mL), H₂O (0.5 mL) and MeOH (1.5 mL) was added NaOH (20.96 mg, 523.98 μmol, 2 eq). The mixture was stirred at 25° C. for 1 hr. The reaction mixture was concentrated under reduced pressure to give the crude product. The crude product was purified by reversed phased HPLC (FA), the target peak was concentrated and then extracted with EA (10 mL*2). The organic layers were dried over anhydrous sodium sulfate, filtered and concentrated to give product. I-41-5 (120 mg, 197.00 μmol, 75.19% yield, 91.69% purity) was obtained as a white solid. LCMS (Method E): R^{t}=0.632 min, [M-Boc+H]⁺=459.2.

### Step 4: Synthesis of I-41-7

To a solution of I-41-5 (60 mg, 107.43 μmol, 1 eq) and I-41-6 (88.21 mg, 118.17 μmol, 1.1 eq) in DMF (0.3 mL) was added EDCI (61.78 mg, 322.28 μmol, 3 eq), HOAt (14.62 mg, 107.43 μmol, 1 eq) and NMM (108.66 mg, 1.07 mmol, 118.11 μL, 10 eq). The mixture was stirred at 25° C. for 1 hr. The mixture was poured into water 10 mL and extracted with EA (10 mL*3). The combined organic layers were concentrated to give a residue. The residue was purified by reversed phased HPLC (FA), the target peak was concentrated and then extracted with EA (10 mL*2). The organic layers were dried over anhydrous sodium sulfate, filtered and concentrated to give I-41-7 (40 mg, 35.16 μmol, 32.73% yield, 100% purity) as yellow oil. LCMS (Method E): R^{t}=0.564 min, [M+H]⁺=1137.7.

### Step 5: Synthesis of I-41-8

To a solution of I-41-7 (34 mg, 29.89 μmol, 1 eq) in DMF (0.3 mL) was added 4-(4-pyridyl)pyridine (2.33 mg, 14.94 μmol, 0.5 eq). Then hypoboric acid (16.08 mg, 179.32 μmol, 6 eq) was added at 0° C. The mixture was stirred at 25° C. for 1 hr. The mixture was poured into water 10 mL and extracted with EA (5 mL*3). The combined organic layers were concentrated to give a residue. The crude product was used into the next step without further purification. I-41-8 (30 mg, 26.27 μmol, 87.90% yield, 97% purity) was obtained as yellow oil. LCMS (Method E): R^{t}=0.484 min, [M+H]⁺=1107.8.

### Step 6: Synthesis of I-41

To a solution of I-41-8 (25 mg, 22.57 μmol, 1 eq) in DCM (1 mL) was added TFA (537.25 mg, 4.71 mmol, 350 μL, 208.76 eq). The mixture was stirred at 25° C. for 1 hr. The reaction mixture was concentrated under reduced pressure to give the crude product. The product was purified by prep-HPLC (column: Phenomenex luna C18 150*25 mm*10 um; mobile phase: [water (TFA)-ACN]; gradient: 10%-40% B over min) and the eluent was concentrated to remove MeCN and then lyophilized. I-41 (15.84 mg, 15.51 μmol, 68.71% yield, 100% purity, TFA salt) was obtained as a yellow solid. LCMS (Method E): R^{t}=0.402 min, [M+H]⁺=907.7. SFC: R^{t}=1.190 min, ee value=100%. ¹H NMR (400 MHZ, METHANOL-d4) δ=8.36 (s, 1H), 7.89-7.81 (m, 2H), 7.43-7.38 (m, 3H), 7.36-7.33 (m, 5H), 7.29-7.26 (m, 1H), 6.89 (d, J=3.6 Hz, 1H), 5.27 (s, 2H), 5.08-4.98 (m, 1H), 4.65-4.59 (m, 2H), 4.25 (s, 2H), 3.92-3.71 (m, 5H), 3.30-3.21 (m, 3H), 3.14-3.10 (m, 1H), 3.04-2.99 (m, 2H), 2.68-2.55 (m, 2H), 2.45-2.22 (m, 5H), 2.16 (d, J=14.8 Hz, 1H), 2.02 (d, J=14.4 Hz, 1H), 1.73-1.52 (m, 5H), 1.43-1.27 (m, 3H). ¹⁹F NMR (377 MHz, METHANOL-d4) δ=−62.705, −76.973, −117.400.

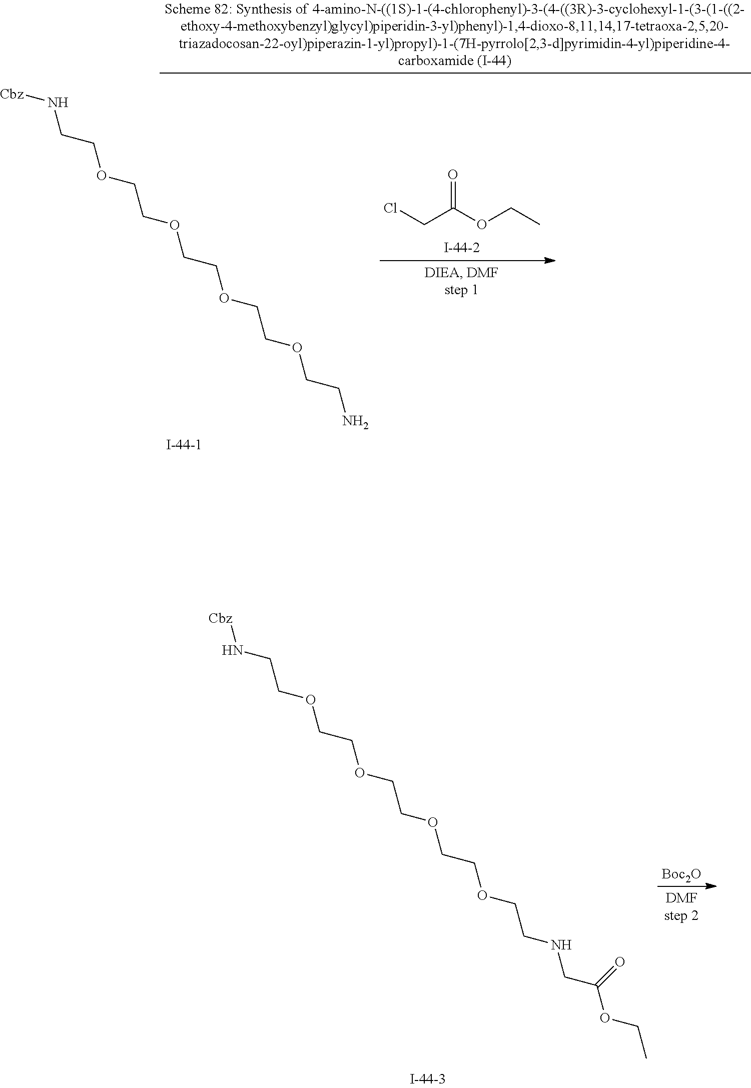

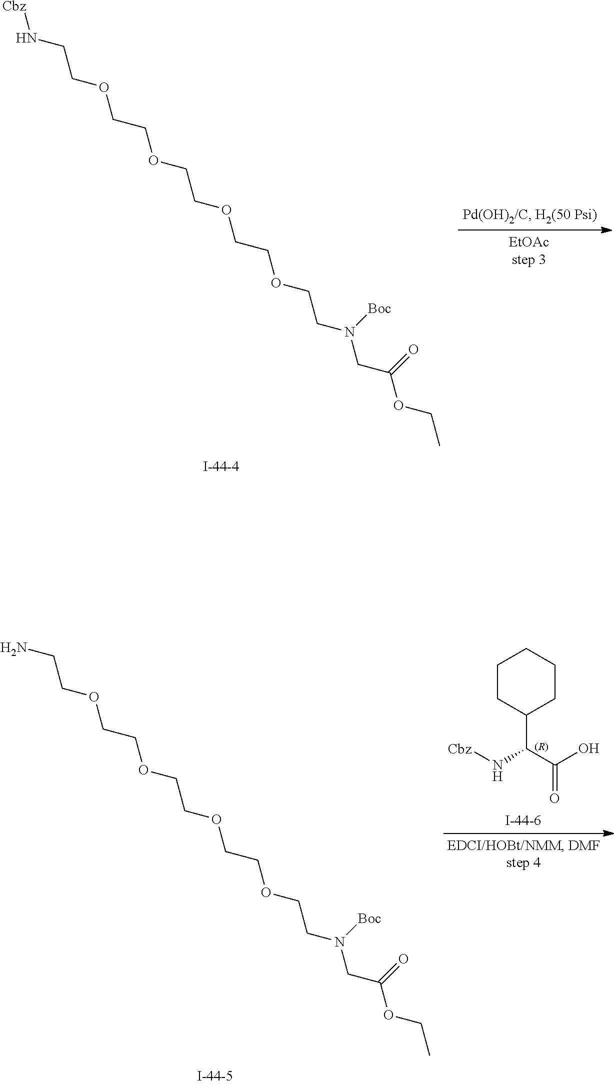

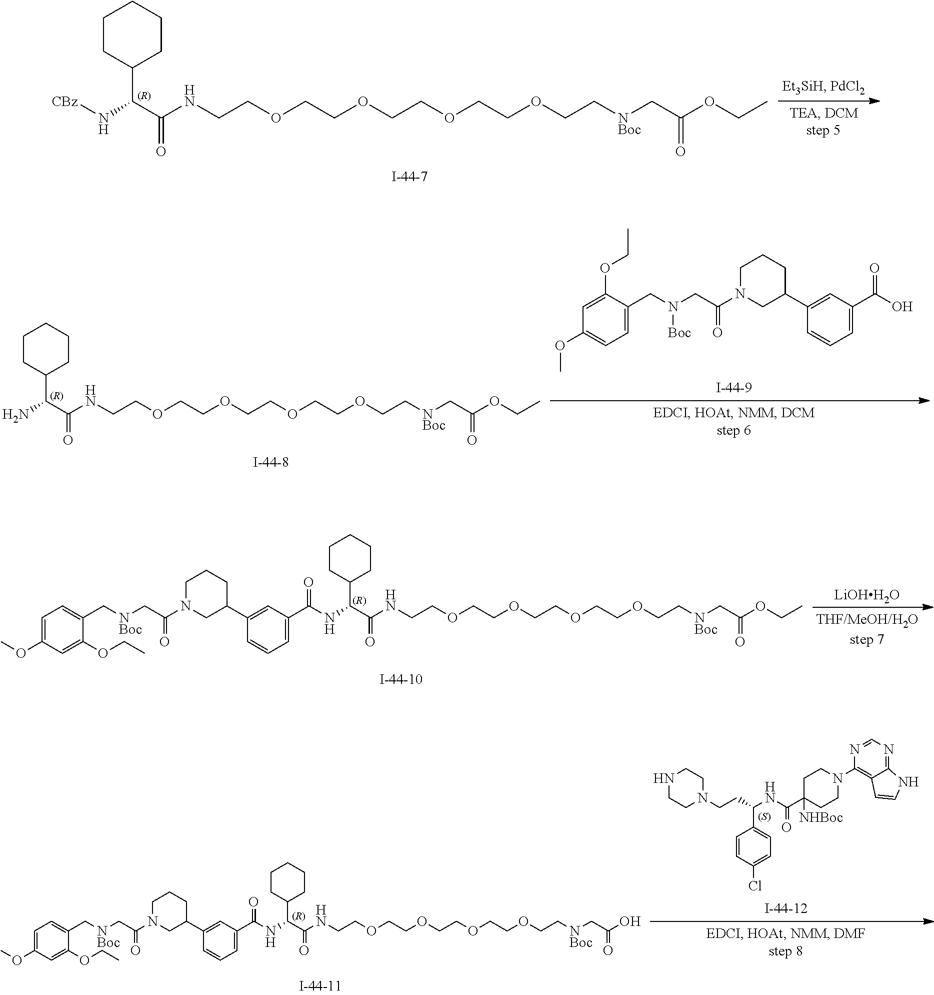

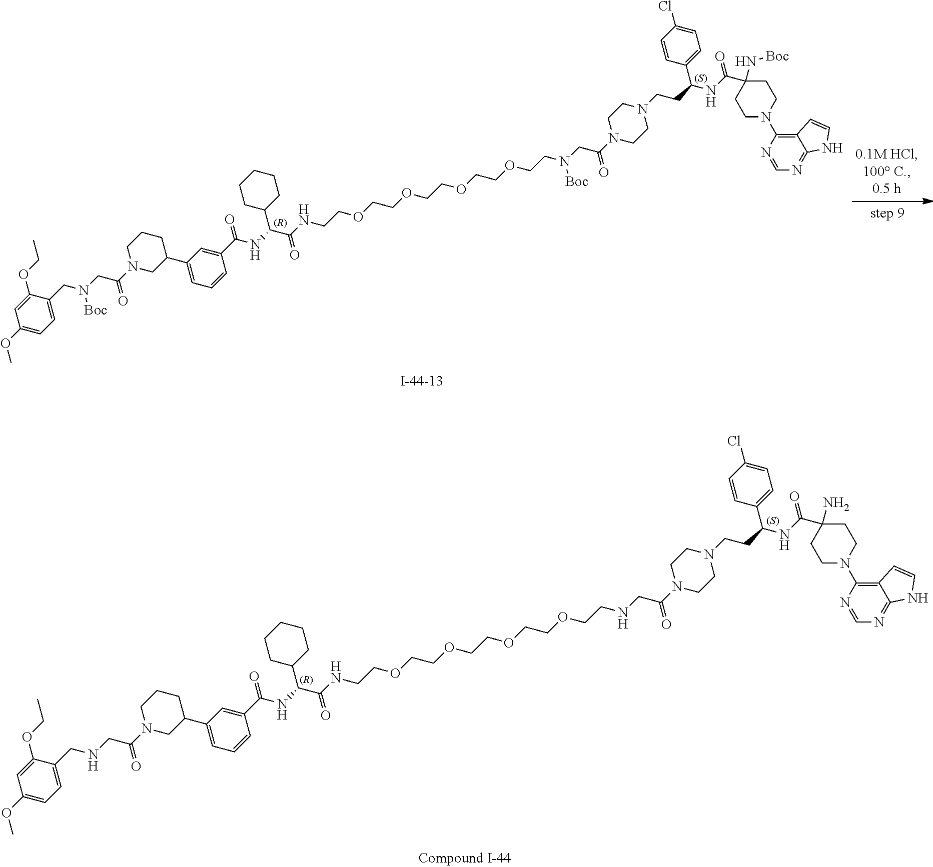

### Step 1: Synthesis of I-44-3

To a solution of I-44-1 (950 mg, 2.56 mmol, 1 eq) and I-44-2 (314.28 mg, 2.56 mmol, 273.29 μL, 1 eq) in DMF (10 mL) was added DIEA (994.34 mg, 7.69 mmol, 1.34 mL, 3 eq). The mixture was stirred at 25° C. for 16 hr. The reaction mixture was used into the next step without further purification. LCMS (Method E): R^{t}=0.421 min, [M+H]⁺=457.3.

### Step 2: Synthesis of I-44-4

To a solution of I-44-3 (1.1 g, 2.41 mmol, 1 eq) in DMF (10 mL) was added Boc₂O (525.87 mg, 2.41 mmol, 553.55 μL, 1 eq). The mixture was stirred at 25° C. for 1 hr. The reaction mixture was quenched with water (10 mL) and extracted with EA (5 mL*3). The organic layers were dried over anhydrous Na₂SO₄, filtered and the filtrate was concentrated under vacuum. The residue was purified by flash silica gel chromatography (ISCO®; 40 g SepaFlash® Silica Flash Column, Eluent of 0˜60% Ethyl acetate/Petroleum ether gradient (40 mL/min). I-44-4 (910 mg, 1.57 mmol, 65.13% yield, 96% purity) was obtained as a white solid. LCMS (Method E): R^{t}=0.556 min, [M+H-Boc]⁺=457.3.

### Step 3: Synthesis of I-44-5

To a suspension of Pd(OH)₂/C (100 mg, 142.42 μmol, 20% purity, 8.71e-2 eq) in EtOAc (5 mL) was added I-44-4 (910 mg, 1.63 mmol, 1 eq) dissolve in EtOAc (5 mL) under N₂. The suspension was degassed under vacuum and purged with H₂ several times. The mixture was stirred under H₂ (50 psi) at 25° C. for 2 hours. The reaction mixture was washed by MeOH 15 mL and then filtered. The filtrate was concentrated to give I-44-5 (600 mg, 1.42 mmol, 86.87% yield) as an off-white oil. ¹H NMR (400 MHZ, CHLOROFORM-d) δ=4.21-4.14 (m, 2H), 4.08-3.96 (m, 2H), 3.70-3.55 (m, 14H), 3.54-3.42 (m, 4H), 2.91-2.83 (m, 2H), 1.44 (d, J=17.6 Hz, 9H), 1.30-1.24 (m, 3H).

### Step 4: Synthesis of I-44-7

To a solution of I-44-5 (600 mg, 1.42 mmol, 1 eq) and I-44-6 (455.10 mg, 1.56 mmol, 1.1 eq) in DMF (5 mL) was added EDCI (544.46 mg, 2.84 mmol, 2 eq), NMM (718.18 mg, 7.10 mmol, 780.63 μL, 5 eq) and HOBt (95.94 mg, 710.04 μmol, 0.5 eq). The mixture was stirred at 25° C. for 1 hr. The reaction mixture was quenched with water (10 mL). The mixture was extracted with EA (5 mL*3) and dried over anhydrous Na₂SO₄. The mixture filtered and the filtrate was concentrated under vacuum. The residue was purified by flash silica gel chromatography (ISCO®; 20 g SepaFlash® Silica Flash Column, Eluent of 60˜100% Ethyl acetate/Petroleum ether gradient @ 20 mL/min). I-44-7 (640 mg, crude) was obtained as a white solid. LCMS (Method E): R^{t}=0.590 min, [M+H]⁺=696.5. ¹H NMR (400 MHZ, DMSO-d₆) δ=7.95 (s, 1H), 7.40-7.27 (m, 5H), 7.19 (d, J=8.8 Hz, 1H), 5.01 (s, 2H), 4.13-4.01 (m, 2H), 3.94 (d, J=8.4 Hz, 2H), 3.86-3.78 (m, 1H), 3.53-3.38 (m, 18H), 3.30-3.08 (m, 3H), 1.72-1.46 (m, 6H), 1.44-1.24 (m, 9H), 1.21-0.99 (m, 7H).

### Step 5: Synthesis of I-44-8

To a solution of I-44-7 (550 mg, 790.41 μmol, 1 eq) in DCM (6 mL) was added PdCl2 (42.05 mg, 237.12 μmol, 0.3 eq), Et₃SiH (735.26 mg, 6.32 mmol, 1.01 mL, 8 eq) and TEA (119.97 mg, 1.19 mmol, 165.02 μL, 1.5 eq). The mixture was stirred at 25° C. for 1 hr. The reaction was diluted with H₂O (5 mL), extracted with ethyl acetate (10 mL+3). The organic phase was washed with brine (5 mL), dried over Na₂SO₄, filtered and concentrated under reduced pressure to give I-44-8 (400 mg, 712.12 μmol, 90.09% yield, crude) colorless oil, and it was used into the next step without further purification. LCMS (Method E): R^{t}=0.460 min, [M+H]⁺=562.5.

### Step 6: Synthesis of I-44-10

To a solution of I-44-8 (400 mg, 712.12 μmol, 1 eq) and I-44-9 (300.01 mg, 569.69 μmol, 0.8 eq) in DCM (5 mL) was added EDCI (409.54 mg, 2.14 mmol, 3 eq), NMM (720.30 mg, 7.12 mmol, 782.94 μL, 10 eq) and HOAt (145.39 mg, 1.07 mmol, 1.5 eq). The mixture was stirred at 25° C. for 1 hr. The reaction was diluted with H₂O (5 mL), extracted with ethyl acetate (10 mL*3). The organic phase was washed with brine (5 mL), dried over Na₂SO₄, filtered, and concentrated under reduced pressure to give a residue. The crude product was purified by reversed-phase HPLC (0.1% FA condition). The eluent was concentrated under reduced pressure to remove ACN, then lyophilized to afford I-44-10 (500 mg, 444.32 μmol, 62.39% yield, 95.112% purity) as colorless oil. LCMS (Method E): R^{t}=0.653 min, [M+H]⁺=1070.7. SFC: R^{t}=1.700 min, 2.163 min.

### Step 7: Synthesis of I-44-11

To a solution of I-44-10 (200 mg, 186.86 μmol, 1 eq) in MeOH (2 mL), THF (2 mL) and H₂O (1 mL) was added LiOH·H₂O (23.52 mg, 560.58 μmol, 3 eq). The mixture was stirred at 25° C. for 1 hr. The reaction was concentrated under reduced pressure to give a residue. The water phase was washed with saturated aqueous citric acid, extracted with EA (4 mL*3). The organic layers were dried over Na₂SO₄, filtered and concentrated under reduced pressure to give I-44-11 (180 mg, 172.70 μmol, 92.42% yield, crude) as a white solid, and it was used into the next step without further purification. LCMS (Method E): R^{t}=0.607 min, [M+H-Boc]⁺=942.6.

### Step 8: Synthesis of I-44-13

To a solution of I-44-11 (180 mg, 172.70 μmol, 1 eq) and I-44-12 (123.75 mg, 207.24 μmol, 1.2 eq) in DMF (2 mL) was added EDCI (99.32 mg, 518.11 μmol, 3 eq), HOAt (23.51 mg, 172.70 μmol, 1 eq) and NMM (174.68 mg, 1.73 mmol, 189.87 μL, 10 eq). The mixture was stirred at 25° C. for 2 hr. The reaction mixture was quenched by addition H₂O 2 mL, and then diluted with EA (2 mL) and extracted with EA (4 mL*3). The combined organic layers were washed with brine (4 mL*3), dried over anhydrous Na₂SO₄, filtered and concentrated under reduced pressure to give a residue. The residue was purified by reversed phase (FA condition), then concentrated to remove organic solvents and lyophilized to give I-44-13 (150 mg, 92.51 μmol, 53.57% yield) as a white solid. LCMS (Method E): R^{t}=0.538 min, [M+H]⁺=1622.9.

### Step 9: Synthesis of I-44

To a solution of I-44-13 (130 mg, 80.18 μmol, 1 eq) in HCl (0.1 M, 4.01 mL, 5 eq). The mixture was stirred at 100° C. for 0.5 hr. The reaction mixture was adjust pH with NH₃·H₂O and extracted with CHCl₃: i-prOH (3:1) 12 mL (4 mL*3). The combined organic layers were dried over anhydrous Na₂SO₄, filtered and concentrated under reduced pressure to give a residue. The residue was purified by prep-HPLC (column: YMC-Actus Triart C18 150*30 mm*7 um; mobile phase: [water (FA)-ACN]; gradient: 10%-40% B over 10 min), then concentrated to remove organic solvents and lyophilized to give I-44 (73.03 mg, 53.42 μmol, 66.63% yield, 100% purity, FA salt) as a white solid. LCMS (Method E): R^{t}=0.420 min, [M+H]⁺=1321.0. SFC: R^{t}=2.296 min, 2.582 min. _I101. ¹H NMR (400 MHZ, METHANOL-d4) δ=8.55 (s, 1H), 8.12 (s, 1H), 7.78-7.68 (m, 2H), 7.48-7.39 (m, 2H), 7.32 (s, 4H), 7.20-7.16 (m, 1H), 7.11 (d, J=3.6 Hz, 1H), 6.60 (d, J=3.6 Hz, 1H), 6.57-6.46 (m, 2H), 4.99-4.96 (m, 1H), 4.56-4.44 (m, 3H), 4.42-4.37 (m, 1H), 4.12-4.03 (m, 2H), 3.96-3.86 (m, 2H), 3.78 (d, J=4.0 Hz, 4H), 3.68-3.53 (m, 24H), 3.49-3.41 (m, 3H), 3.39-3.33 (m, 1H), 3.18-3.05 (m, 1H), 2.90-2.86 (m, 2H), 2.83-2.65 (m, 2H), 2.49-2.29 (m, 6H), 2.24-2.10 (m, 2H), 2.06-1.92 (m, 3H), 1.88-1.65 (m, 8H), 1.64-1.51 (m, 3H), 1.46-1.38 (m, 3H), 1.31-1.06 (m, 5H).

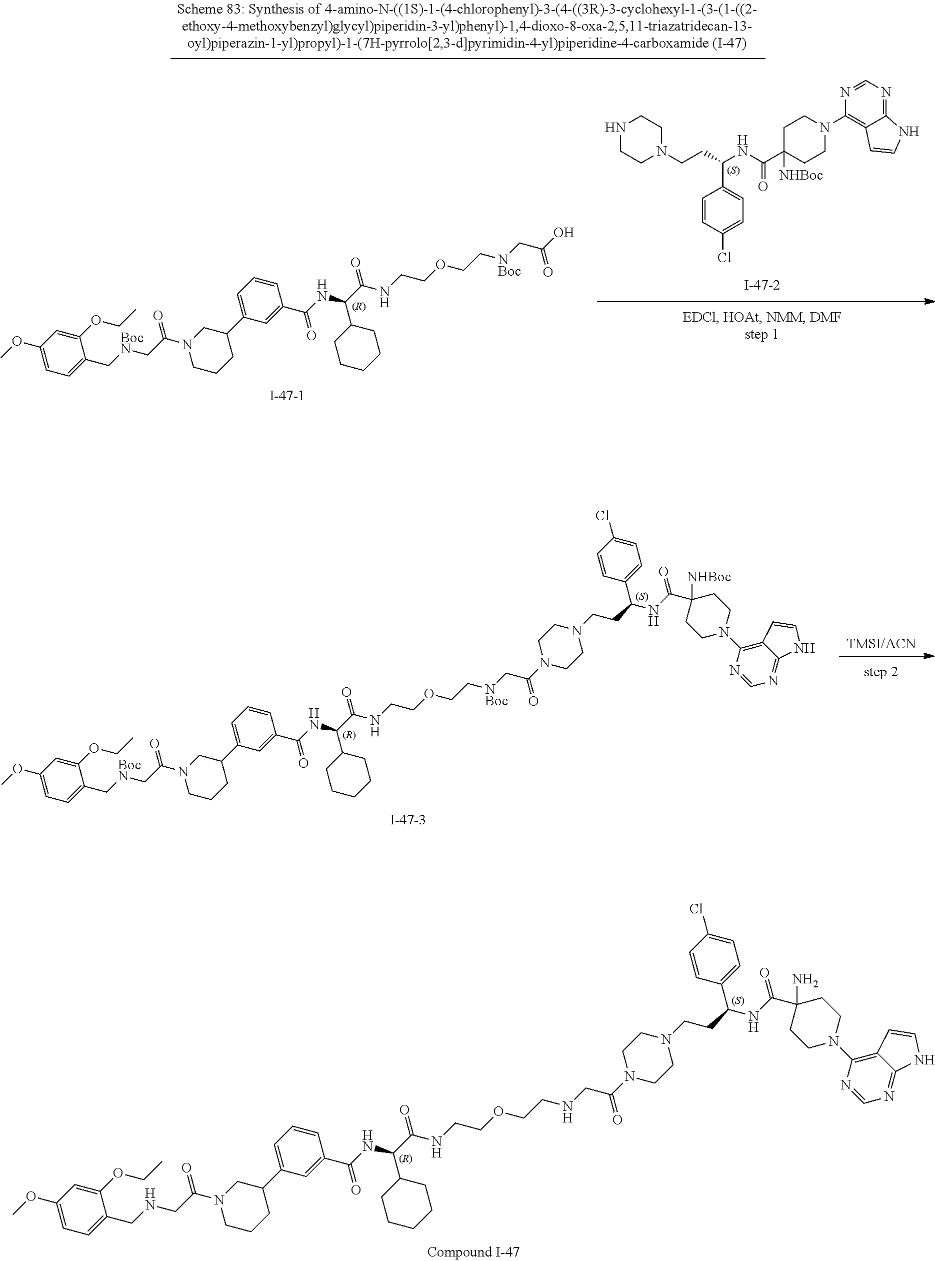

### Step 1: Synthesis of I-47-3

To a solution of I-47-1 (65 mg, 71.42 μmol, 1 eq), EDCI (27.38 mg, 142.84 μmol, 2 eq), HOAt (4.86 mg, 35.71 μmol, 0.5 eq), NMM (36.12 mg, 357.10 μmol, 39.26μ, 5 eq) in DMF (1 mL) was added I-47-2 (51.18 mg, 85.70 μmol, 1.2 eq). Then the mixture was stirred at 25° C. for 1 hr. The reaction mixture was poured into water (5 mL) and extracted with DCM (5 mL*2), the organic layer was washed with brine (5 mL) and dried over Na₂SO₄. Concentrated to get the crude product. The crude product was purified by flash silica gel column chromatography (SiO₂, PE:EA=10:1 to 3:1) to give I-47-3 (85 mg, 39.33 μmol, 55.06% yield, 68.9% purity) as a yellow solid. LCMS (Method G): R^{t}=0.833 min, M+H=1489.2.

### Step 2: Synthesis of I-47

To a solution of I-47-3 (80 mg, 53.72 μmol, 1 eq) in MeCN (1 mL) was added TMSI (107.49 mg, 537.19 μmol, 73.12 μL, 10 eq). Then the mixture was stirred at 0° C. for 0.5 hr. The reaction mixture was concentrated under vacuum to get the crude product. The crude product was purified by prep-HPLC (column: Phenomenex Luna C18 150*30 mm*5 um; mobile phase: [water (FA)-ACN]; gradient: 8%-38% B over 9 min) to give I-47 (24.95 mg, 20.12 μmol, 37.46% yield, 99.60% purity, FA) as a white solid. LCMS (Method E): R^{t}=0.421 min, [M+H]⁺=1188.7. SFC: R^{t}=4.218 min, 8.485 min. ¹H NMR (400 MHZ, METHANOL-d4) δ=8.46 (br s, 3H), 8.16 (s, 1H), 7.84-7.71 (m, 2H), 7.56-7.40 (m, 2H), 7.39-7.26 (m, 5H), 7.17 (d, J=3.6 Hz, 1H), 6.74-6.53 (m, 3H), 5.02-5.00 (m, 1H), 4.56 (br d, J=14.0 Hz, 3H), 4.33 (br d, J=8.0 Hz, 1H), 4.26-3.91 (m, 8H), 3.86-3.55 (m, 12H), 3.54-3.38 (m, 4H), 3.31-3.12 (m, 3H), 2.93-2.65 (m, 2H), 2.57-2.19 (m, 8H), 2.04 (br d, J=8.4 Hz, 3H), 1.94-1.56 (m, 11H), 1.53-1.41 (m, 3H), 1.39-1.07 (m, 5H).

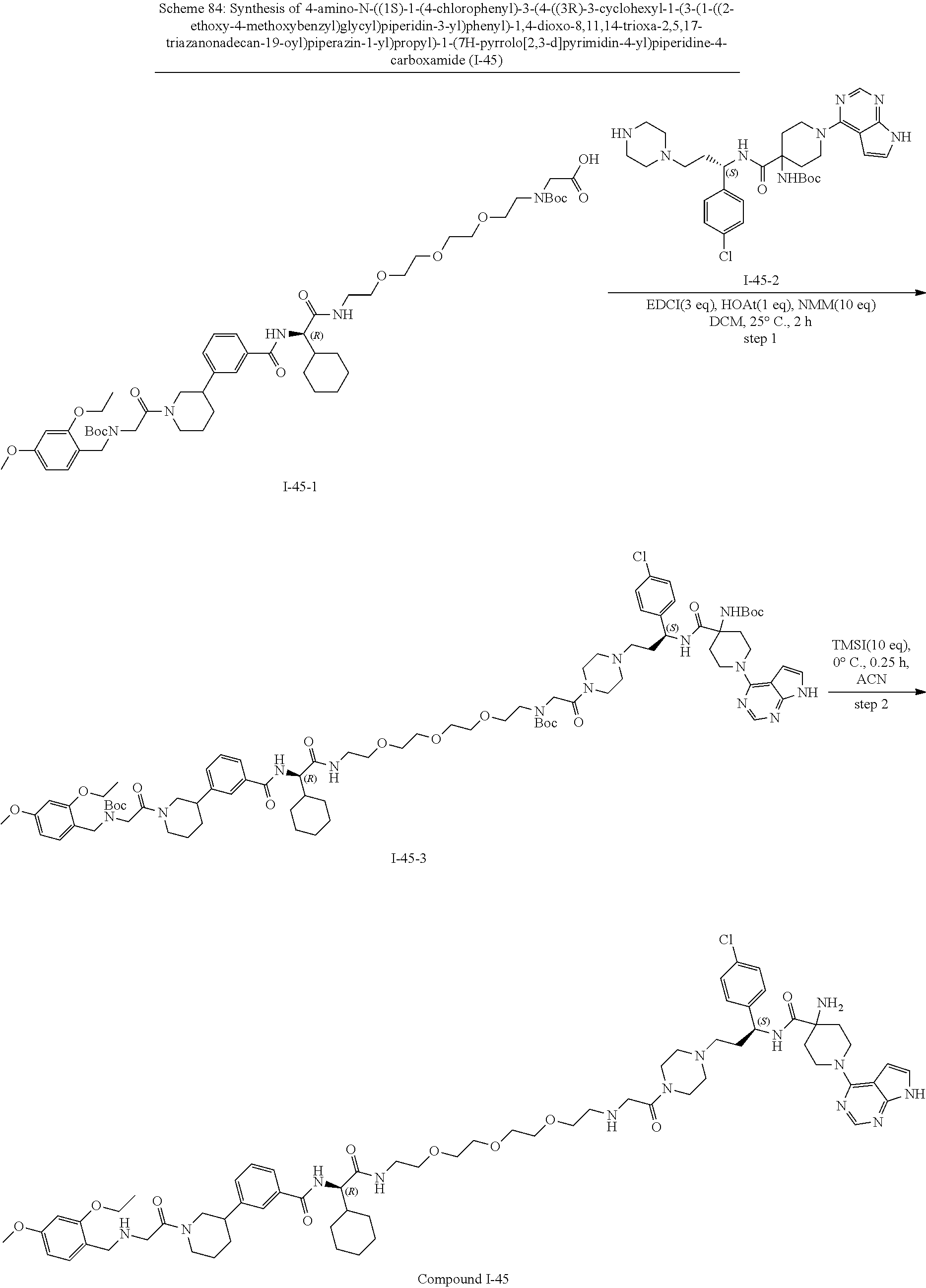

### Step 1: Synthesis of I-45-3

To a solution of I-45-3 (80 mg, 80.14 μmol, 1 eq) and I-45-2 (71.79 mg, 96.17 μmol, 1.2 eq) in DCM (1.5 mL) was added EDCI (46.09 mg, 240.43 μmol, 3 eq), HOAt (10.91 mg, 80.14 μmol, 1 eq) and NMM (81.06 mg, 801.44 μmol, 88.11 μL, 10 eq). The mixture was stirred at 25° C. for 2 h. The reaction mixture was quenched by addition H₂O (1.5 mL), and then diluted with DCM (1 mL) and extracted with DCM (2 mL*3). The combined organic layers were dried over anhydrous Na₂SO₄, filtered and concentrated under reduced pressure to give a residue. The residue was purified by reversed phase (NH₃·H₂O condition), then concentrated to remove organic solvents and lyophilized to give I-45-3 (50 mg, 31.70 μmol, 39.55% yield) as a white solid. LCMS (Method E): R^{t}=0.579 min, [M+H]⁺=1577.9.

### Step 2: Synthesis of I-45

To a solution of I-45-3 (40 mg, 25.36 μmol, 1 eq) in ACN (1 mL) was added TMSI (50.74 mg, 253.59 μmol, 34.52 μL, 10 eq). The mixture was stirred at 0° C. for 0.25 h. The reaction mixture was quenched by addition H₂O (1 mL) at 0° C., and then adjust pH˜8 with saturated NaHCO₃ solution, extracted with EA (2 mL*3). The combined organic layers were dried over anhydrous Na₂SO₄, filtered and concentrated under reduced pressure to give a residue. The residue was purified by prep-HPLC (column: Waters Xbridge 150*25 mm*5 um; mobile phase: [water (ammonia hydroxide v/v)-ACN]; gradient: 40%-70% B over min), then concentrated to remove organic solvents and lyophilized to give I-45 (16.55 mg, 12.69 μmol, 50.04% yield, 97.91% purity) as a white solid. LCMS (Method E): R^{t}=0.439 min, [M+H]⁺=1276.8. ¹H NMR (400 MHZ, METHANOL-d4) δ=8.12 (s, 1H), 7.77-7.68 (m, 2H), 7.46-7.39 (m, 2H), 7.31 (d, J=1.6 Hz, 3H), 7.15-7.09 (m, 2H), 6.61 (d, J=3.6 Hz, 1H), 6.54-6.42 (m, 2H), 4.96 (d, J=6.4 Hz, 1H), 4.55-4.44 (m, 3H), 4.41-4.37 (m, 1H), 4.08-4.00 (m, 2H), 3.88-3.68 (m, 7H), 3.67-3.53 (m, 16H), 3.50-3.42 (m, 6H), 3.41-3.34 (m, 2H), 3.15-3.03 (m, 1H), 2.78-2.76 (m, 2H), 2.75-2.60 (m, 2H), 2.50-2.26 (m, 6H), 2.23-2.09 (m, 2H), 2.08-1.91 (m, 3H), 1.89-1.67 (m, 8H), 1.64-1.50 (m, 3H), 1.46-1.37 (m, 3H), 1.32-1.08 (m, 5H). SFC: R^{t}=2.543 min, R^{t}=4.694 min. _DN1201.

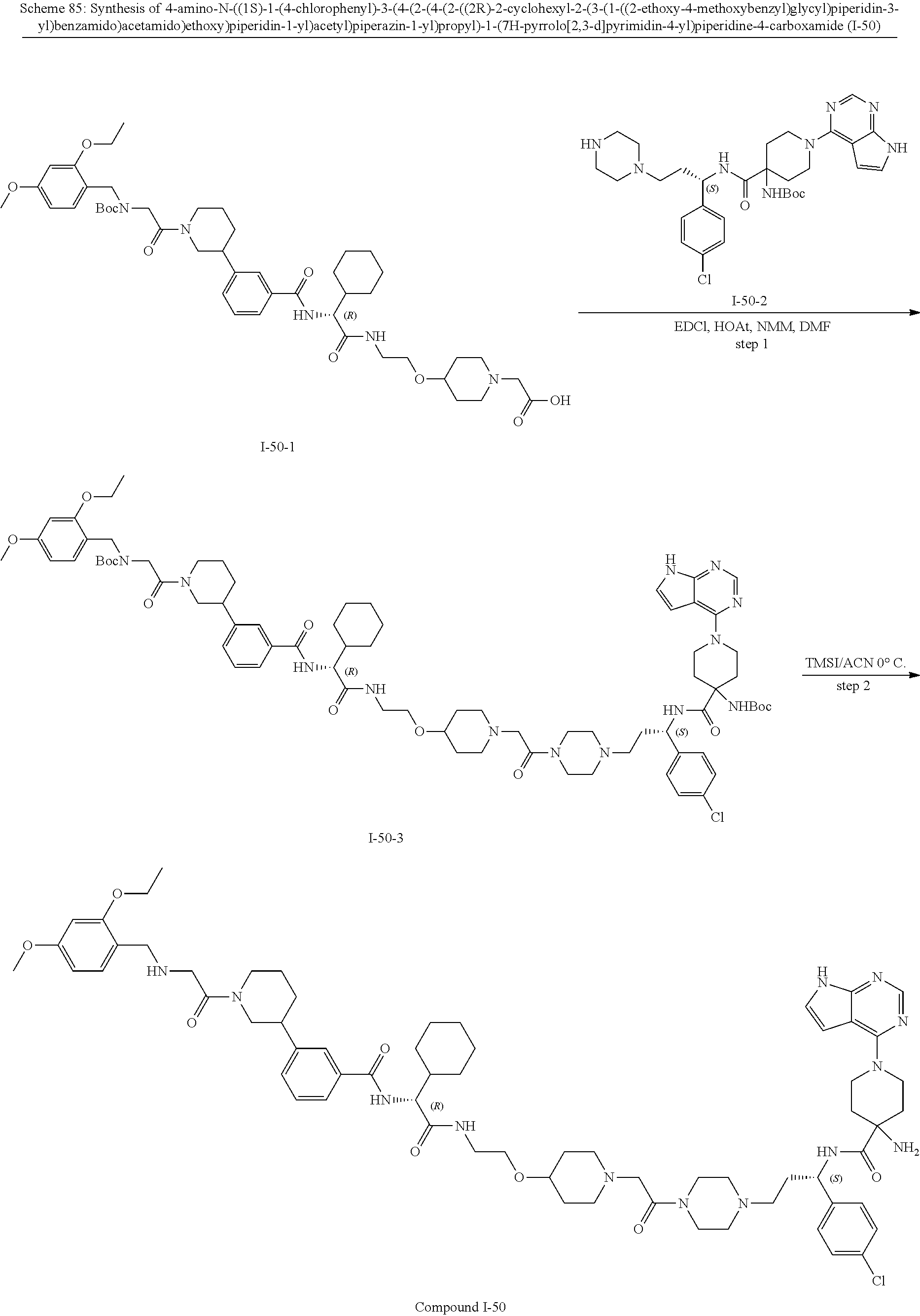

### Step 1: Synthesis of I-50-3

To a solution of I-50-1 (115 mg, 135.29 μmol, 1 eq) in DMF (1.5 mL) was added EDCI (77.80 mg, 405.86 μmol, 3 eq), HOAt (27.62 mg, 202.93 μmol, 1.5 eq) and NMM (68.42 mg, 676.43 μmol, 74.37 μL, 5 eq), then I-50-2 (121.18 mg, 162.34 μmol, 1.2 eq) was added in. The mixture was stirred at 25° C. for 12 hr. The reaction was poured to water (2 ml), extracted with EA (2 ml*3) and the organic layers were concentrated under vacuum. The residue was purified by flash silica gel chromatography (ISCO®; 4 g SepaFlash® Silica Flash Column, Eluent of 0˜50% EA/MeOH gradient @ 18 mL/min). I-50-4 (100 mg, 69.97 μmol, 51.72% yield) was obtained as yellow oil. LCMS (Method E): R^{t}=0.514 min, [M+H]⁺=1429.8.

### Step 2: Synthesis of I-50

To a solution of I-50-3 (60 mg, 41.98 μmol, 1 eq) in ACN (1 mL) was added TMSI (84.00 mg, 419.82 μmol, 57.14 μL, 10 eq) at 0° C. The mixture was stirred at 0° C. for 0.5 hr. The reaction mixture was poured to water (2 ml), extracted with (CHCl₃: i-PrOH=5:1) (2 ml*3) and the organic layers were concentrated under vacuum. The residue was purified by prep-HPLC (basic condition column: Waters Xbridge 150*25 mm*5 um; mobile phase: [water (NH₄HCO₃)-ACN]; gradient: 38%-68% B over 9 min) to give I-50 (7.65 mg, 5.95 μmol, 14.17% yield, 95.578% purity) as an off-white solid. LCMS (Method G): R^{t}=0.754 min, [M+H]⁺=1228.8. SFC: R^{t}=2.537 min, 3.818 min. ¹H NMR (400 MHZ, METHANOL-d4) δ=8.12 (s, 1H), 7.77-7.65 (m, 2H), 7.51-7.40 (m, 2H), 7.32 (s, 4H), 7.17-7.05 (m, 2H), 6.61 (d, J=3.6 Hz, 1H), 6.55-6.38 (m, 2H), 5.02-4.92 (m, 3H), 4.56-4.42 (m, 4H), 4.38-4.37 (m, 1H), 4.10-3.99 (m, 2H), 3.92-3.79 (m, 1H), 3.79-3.73 (m, 4H), 3.71 (d, J=5.6 Hz, 1H), 3.70-3.66 (m, 1H), 3.67-3.62 (m, 2H), 3.60 (d, J=4.4 Hz, 2H), 3.53 (s, 2H), 3.49-3.37 (m, 3H), 3.18-3.01 (m, 3H), 2.79-2.61 (m, 4H), 2.45-2.44 (m, 3H), 2.39-2.25 (m, 3H), 2.22-2.12 (m, 3H), 2.11-1.93 (m, 3H), 1.91-1.79 (m, 6H), 1.79-1.71 (m, 3H), 1.69-1.69 (m, 1H), 1.69-1.61 (m, 1H), 1.55 (d, J=13.2 Hz, 4H), 1.47-1.33 (m, 4H), 1.33-1.20 (m, 3H), 1.19-1.07 (m, 2H).

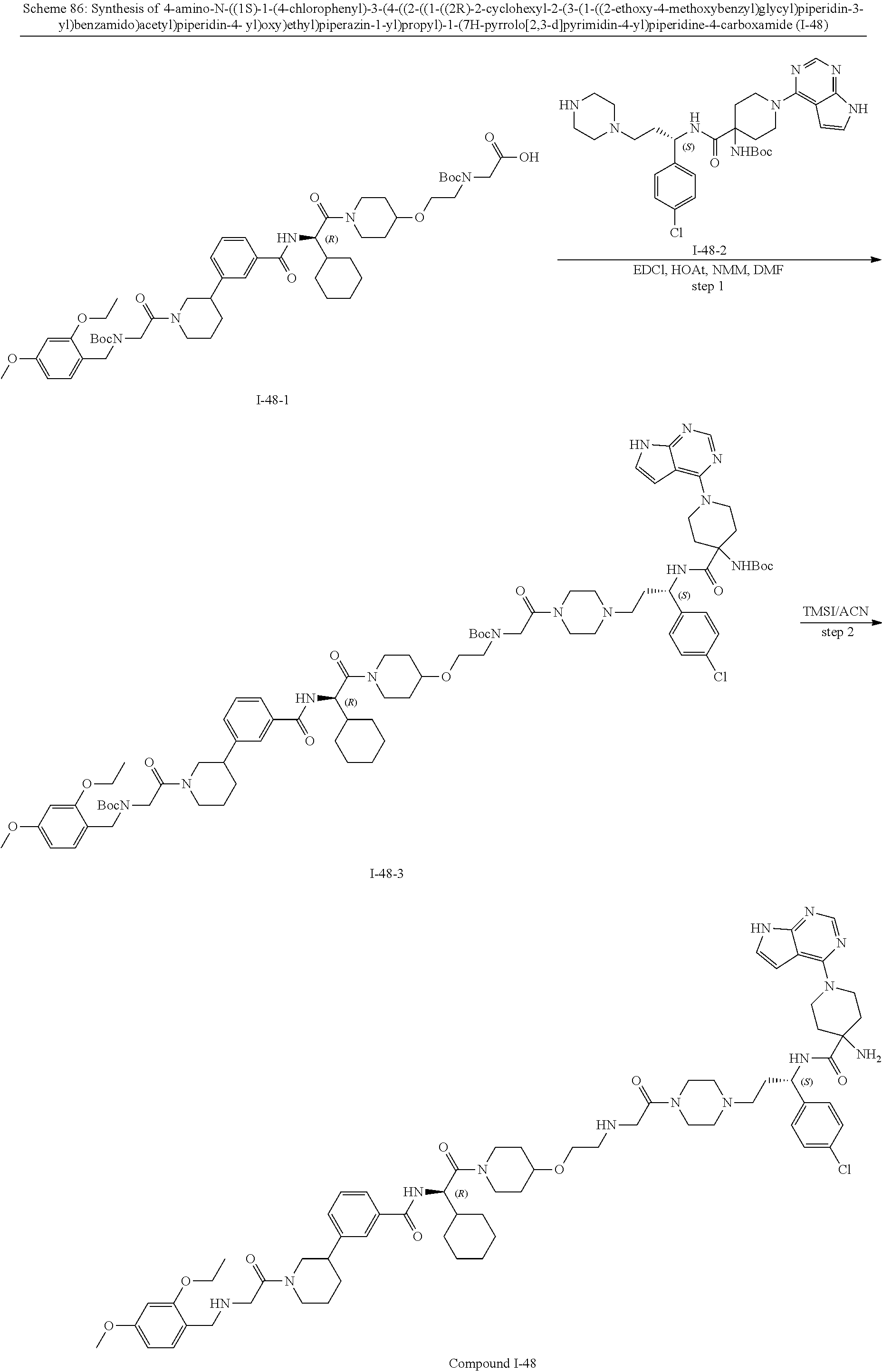

### Step 1: Synthesis of I-48-3

To a solution of I-48-1 (65 mg, 68.41 μmol, 1 eq), EDCI (26.23 mg, 136.82 μmol, 2 eq), HOAt (4.66 mg, 34.20 μmol, 0.5 eq), NMM (34.60 mg, 342.05 μmol, 37.61 μL, 5 eq) in DMF (0.5 mL) was added I-48-2 (49.02 mg, 82.09 μmol, 1.2 eq). Then the mixture was stirred at 25° C. for 1 hr. The reaction mixture was poured into water (5 mL) and extracted with DCM (5 mL*2), the organic layer was washed with brine (5 mL) and dried over Na₂SO₄. Concentrated to get the crude product. The crude product was purified by flash silica gel column chromatography (SiO₂, PE:EA=10:1 to 3:1) to give I-48-3 (85 mg, 38.80 μmol, 56.71% yield, 69.8% purity) as a yellow solid. LCMS (Method G): R^{t}=0.854 min, [M+H]⁺=1529.2.

### Step 2: Synthesis of I-48

To a solution of I-48-3 (80 mg, 52.31 μmol, 1 eq) in MeCN (1 mL) was added TMSI (104.67 mg, 523.12 μmol, 71.21 μL, 10 eq). Then the mixture was stirred at 0° C. for 0.5 hr. The reaction mixture was concentrated under vacuum to get the crude product. The crude product was purified by prep-HPLC (column: Phenomenex luna C18 150*25 mm*10 um; mobile phase: [water (FA)-ACN]; gradient: 8%-38% B over 9 min) to get I-48 (12.68 mg, 9.89 μmol, 18.90% yield, 99.40% purity, FA salt) as a white solid. LCMS (Method E): R^{t}=0.430 min, [M+H]⁺=1228.7. SFC: R^{t}=4.631 min, 6.549 min. ¹H NMR (400 MHZ, METHANOL-d4) δ=8.49 (br s, 3H), 8.15 (d, J=2.4 Hz, 1H), 7.87-7.67 (m, 2H), 7.60-7.42 (m, 2H), 7.40-7.23 (m, 5H), 7.21-7.06 (m, 1H), 6.75-6.47 (m, 3H), 5.08-4.98 (m, 2H), 4.65-4.47 (m, 3H), 4.32-3.87 (m, 10H), 3.87-3.79 (m, 5H), 3.79-3.50 (m, 8H), 3.50-3.39 (m, 2H), 3.24 (br s, 3H), 2.98-2.66 (m, 2H), 2.59-2.32 (m, 6H), 2.30-2.15 (m, 2H), 2.12-1.84 (m, 9H), 1.83-1.56 (m, 9H), 1.53-1.41 (m, 3H), 1.35-1.02 (m, 5H).

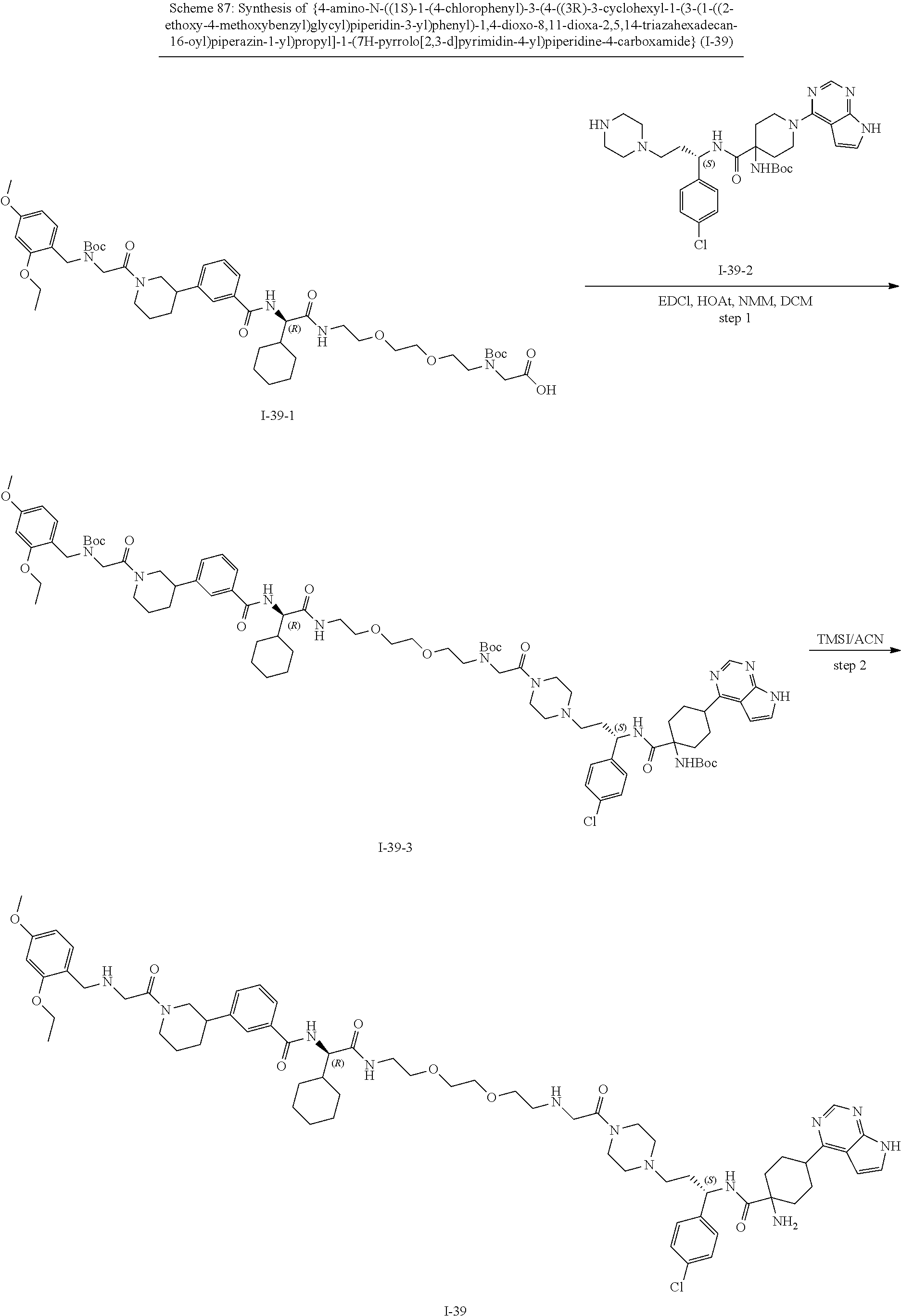

### Step 1: Synthesis of I-39-3

To a mixture of I-39-1 (100 mg, 79.65 μmol, 1 eq), EDCI (45.81 mg, 238.96 μmol, 3 eq) and HOAt (10.84 mg, 79.65 μmol, 1 eq) in DCM (2 mL) was added NMM (40.28 mg, 398.26 μmol, 43.79 μL, 5 eq), the mixture was stirred at 20° C. for 10 min. Then I-39-2 (59.46 mg, 79.65 μmol, 1 eq) was added. The mixture was stirred at 20° C. for 1 hr. The mixture was purified by flash silica gel chromatography (ISCO®; 12 g SepaFlash® Silica Flash Column, Eluent of 0˜30% MeOH/Ethyl acetate gradient @ 36 mL/min) and the eluent was concentrated to give I-39-3 (70 mg, 36.07 μmol, 45.28% yield, 79% purity) as yellow oil. LCMS (Method G): R^{t}=0.825 min, [M+H]⁺=1533.1.

### Step 2: Synthesis of I-39

To a solution of I-39-3 (50 mg, 25.76 μmol, 1 eq) in ACN (1.5 mL) was added TMSI (51.55 mg, 257.62 μmol, 35.07 μL, 10 eq) at 0° C., and then the mixture was stirred at 0° C. for 15 min. The mixture was poured into water 1 mL and adjusted to pH=9 with the solution of saturated NaHCO₃ and then extracted with CHCl3/isopropanol (3 mL*3). The combined organic layers were dried over anhydrous Na₂SO₄, filtered and concentrated to give a residue. The residue was purified by prep-HPLC (column: Waters Xbridge 150*25 mm*5 um; mobile phase: [water (ammonia hydroxide v/v)-ACN]; gradient: 38%-68% B over min) and the eluent was concentrated to remove MeCN and then lyophilized. I-39 (13.65 mg, 11.07 μmol, 42.98% yield, 100% purity) was obtained as a white solid. LCMS (Method E): R^{t}=0.447 min, [M+Na]⁺=1254.9. SFC: R^{t}=3.607 & 5.863 min. ¹H NMR (400 MHZ, METHANOL-d4) δ=8.12 (s, 1H), 7.81-7.64 (m, 2H), 7.50-7.37 (m, 2H), 7.36-7.28 (m, 4H), 7.19-7.08 (m, 2H), 6.60 (d, J=3.6 Hz, 1H), 6.55-6.42 (m, 2H), 4.99-4.95 (m, 1H), 4.57-4.43 (m, 3H), 4.42-4.37 (m, 1H), 4.13-3.99 (m, 2H), 3.86-3.72 (m, 6H), 3.70-3.41 (m, 19H), 3.39-3.34 (m, 1H), 3.18-3.01 (m, 1H), 2.85-2.58 (m, 4H), 2.53-2.25 (m, 6H), 2.24-2.08 (m, 2H), 2.07-1.90 (m, 3H), 1.90-1.63 (m, 8H), 1.63-1.49 (m, 3H), 1.48-1.36 (m, 3H), 1.34-1.06 (m, 5H).

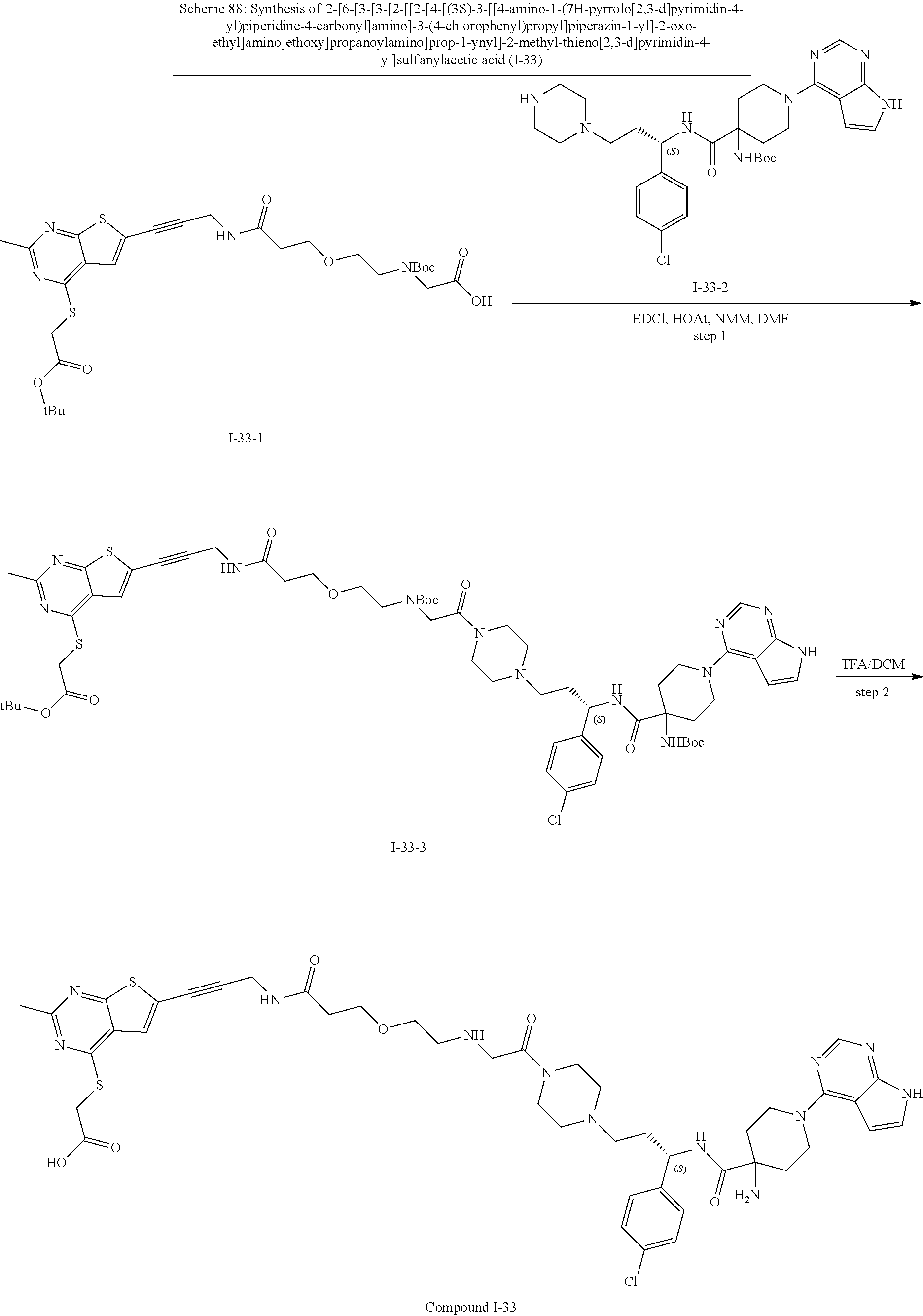

### Step 1: Synthesis of I-33

To a solution of I-33-1 (7 mg, 11.24 μmol, 1 eq) and I-33-2 (6.71 mg, 11.24 μmol, 1 eq) in DMF (0.5 mL) was added EDCI (4.31 mg, 22.48 μmol, 2 eq) NMM (5.68 mg, 56.20 μmol, 6.18 μL, 5 eq) and HOAt (764.97 μg, 5.62 μmol, 0.5 eq). The mixture was stirred at 25° C. for 4 hr. The reaction mixture was quenched with water (5 mL). The mixture was extracted with EA (5 mL*3) and dried over anhydrous Na₂SO₄. The mixture was filtered and the filtrate was concentrated under vacuum to give I-33-3 (12 mg, crude) as a white solid, which was used for next step without further purification. The mixture was concentrated under pressure to give crude product. The residue was purified by prep-HPLC (column: Waters Xbridge 150*25 mm*5 um; mobile phase: [water (NH4HCO3)-ACN]; gradient: 10%-40% B over 10 min). I-33 (2.19 mg, 2.29 μmol, 22.97% yield, 99% purity) was obtained as a white solid. LCMS (Method E): R^{t}=0.404 min, [M+H]⁺=945.5. SFC: R^{t}=0.577 min. ¹H NMR (400 MHZ, DMSO-d₆) δ=11.68 (s, 1H), 9.19-8.95 (m, 1H), 8.59-8.46 (m, 1H), 8.12 (s, 1H), 7.58 (s, 1H), 7.39-7.30 (m, 4H), 7.20-7.09 (m, 1H), 6.58 (d, J=3.6 Hz, 1H), 4.88-4.77 (m, 1H), 4.22-4.19 (m, 2H), 4.06-3.95 (m, 2H), 3.76-3.64 (m, 2H), 3.63-3.59 (m, 2H), 3.46 (s, 2H), 3.41 (s, 4H), 2.71-2.66 (m, 2H), 2.58 (s, 2H), 2.39-2.32 (m, 4H), 2.28-2.18 (m, 6H), 2.11-1.95 (m, 4H), 1.87-1.80 (m, 2H), 1.47 (s, 3H).

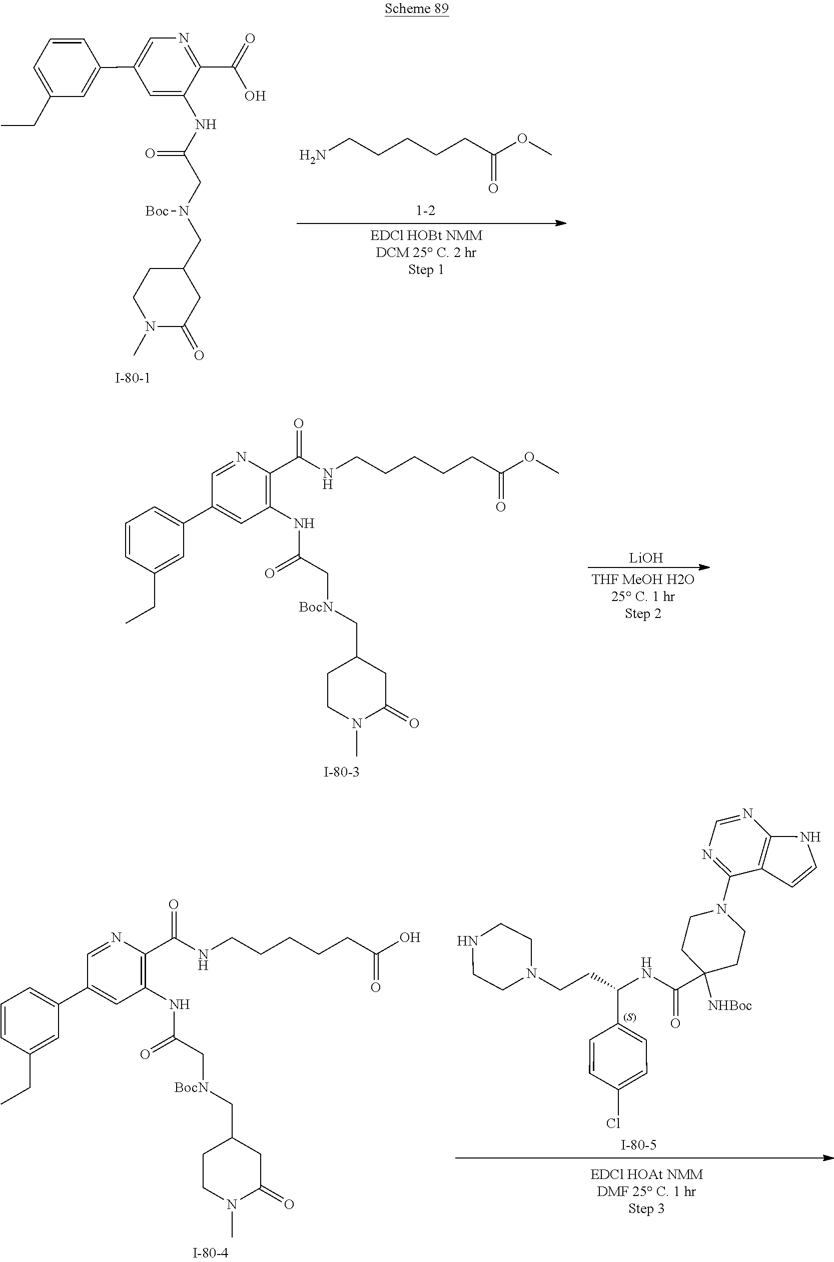

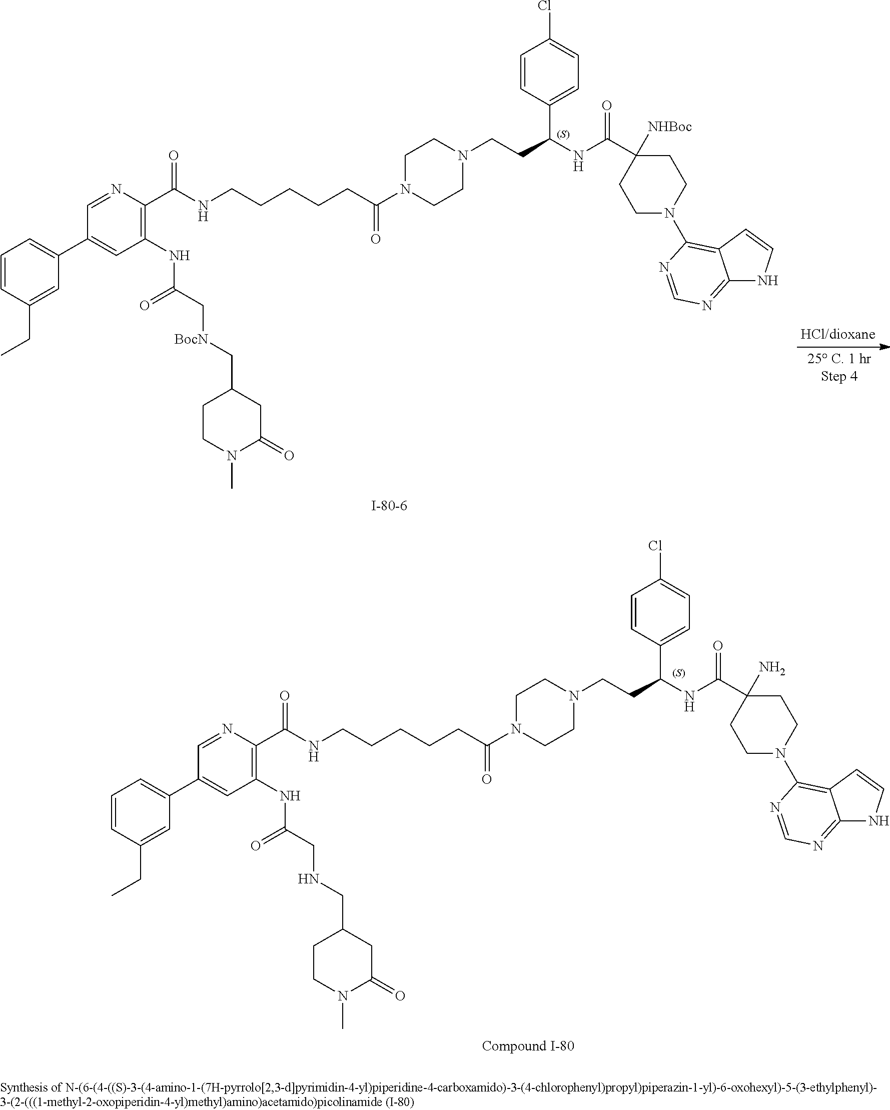

### Step 1: Synthesis of I-80-3

To a solution of 3 I-80-1 (100 mg, 190.62 μmol, 1 eq) in DCM (1 mL) was added HOBt (12.88 mg, 95.31 μmol, 0.5 eq), EDCI (73.08 mg, 381.24 μmol, 2 eq), NMM (96.40 mg, 953.09 μmol, 104.79 μL, 5 eq) and I-80-2 (38.09 mg, 209.68 μmol, 1.1 eq, HCl). The mixture was stirred at 25° C. for 2 hr. The reaction mixture was quenched by addition water 5 mL, and then extracted with EA (5 mL*3). Then dried over Na₂SO₄, filtered and concentrated under reduced pressure to give a residue. The crude product was used into the next step without further purification. I-80-3 (100 mg, crude) was obtained as a yellow solid. LCMS (Method E): R^{t}=0.651 min, [M+H]⁺=652.4.

### Step 2: Synthesis of I-80-4

To a solution of I-80-3 (80 mg, 122.74 μmol, 1 eq) in THF (1 mL), H₂O (1 mL) and MeOH (0.5 mL) was added LiOH·H₂O (15.45 mg, 368.22 μmol, 3 eq). The mixture was stirred at 25° C. for 1 hr. The mixture poured into water 10 mL, then adjusted to pH<6 by in citric acid to and extracted with EA (10 mL*3). Then dried over Na₂SO₄, filtered and concentrated under reduced pressure to give a residue. I-80-4 (79 mg, crude) was obtained as colorless oil. LCMS: R^{t}=0.622 min, [M+H]⁺=638.5.

### Step 3: Synthesis of I-80-6

To a solution of I-80-4 (79 mg, 123.87 μmol, 1 eq) in DMF (1 mL) was added EDCI (47.49 mg, 247.74 μmol, 2 eq), NMM (62.65 mg, 619.35 μmol, 68.09 μL, 5 eq), HOAt (8.43 mg, 61.94 μmol, 0.5 eq) and I-80-5 (88.76 mg, 148.64 μmol, 1.2 eq). The mixture was stirred at 25° C. for 1 hr. The reaction mixture was quenched by addition water 20 mL, and then extracted with EA (5 mL*3). Then dried over Na₂SO₄, filtered and concentrated under reduced pressure to give a residue. The residue was purified by prep-HPLC (column: C18 150×30 mm; mobile phase: [water (FA)-ACN]; gradient: 32%-62% B over 7 min), the target peak was concentrated and lyophilized to afford desired product. I-80-6 (70 mg, 57.52 μmol, 46.44% yield) was obtained as a yellow solid. LCMS (Method E): R^{t}=0.524 min, [M+H]⁺=1216.0.

### Step 4: Synthesis of 1-80

To a solution of I-80-6 (30 mg, 24.65 μmol, 1 eq) in DCM (0.3 mL) was added HCl/dioxane (4 M, 0.3 mL, 48.68 eq). The mixture was stirred at 25° C. for 1 hr. The reaction mixture was concentrated under reduced pressure to give 1-80 (35.17 mg, 33.34 μmol, 67.61% yield, 99.82% purity, HCl salt) as a white solid. LCMS (Method E): R^{t}=0.421 min, [M+H]⁺=1016.7. SFC: R^{t}=3.201 min. ¹H NMR (400 MHz, METHANOL-d4) δ=9.21 (s, 1H), 8.63 (s, 1H), 8.38 (s, 1H), 7.54-7.38 (m, 6H), 7.37-7.29 (m, 3H), 7.01 (s, 1H), 5.06-5.05 (d, J=3.6 Hz, 1H), 4.69-4.51 (m, 3H), 4.31-4.13 (m, 3H), 4.02-4.00 (d, J=10.0 Hz, 2H), 3.74-3.54 (m, 3H), 3.46-3.44 (d, J=8.0 Hz, 5H), 3.29-3.03 (m, 6H), 2.98 (s, 3H), 2.87-2.63 (m, 5H), 2.60-2.38 (m, 4H), 2.37-2.22 (m, 3H), 2.20-2.01 (m, 2H), 1.68 (s, 5H), 1.46 (s, 2H), 1.29 (m, 3H).

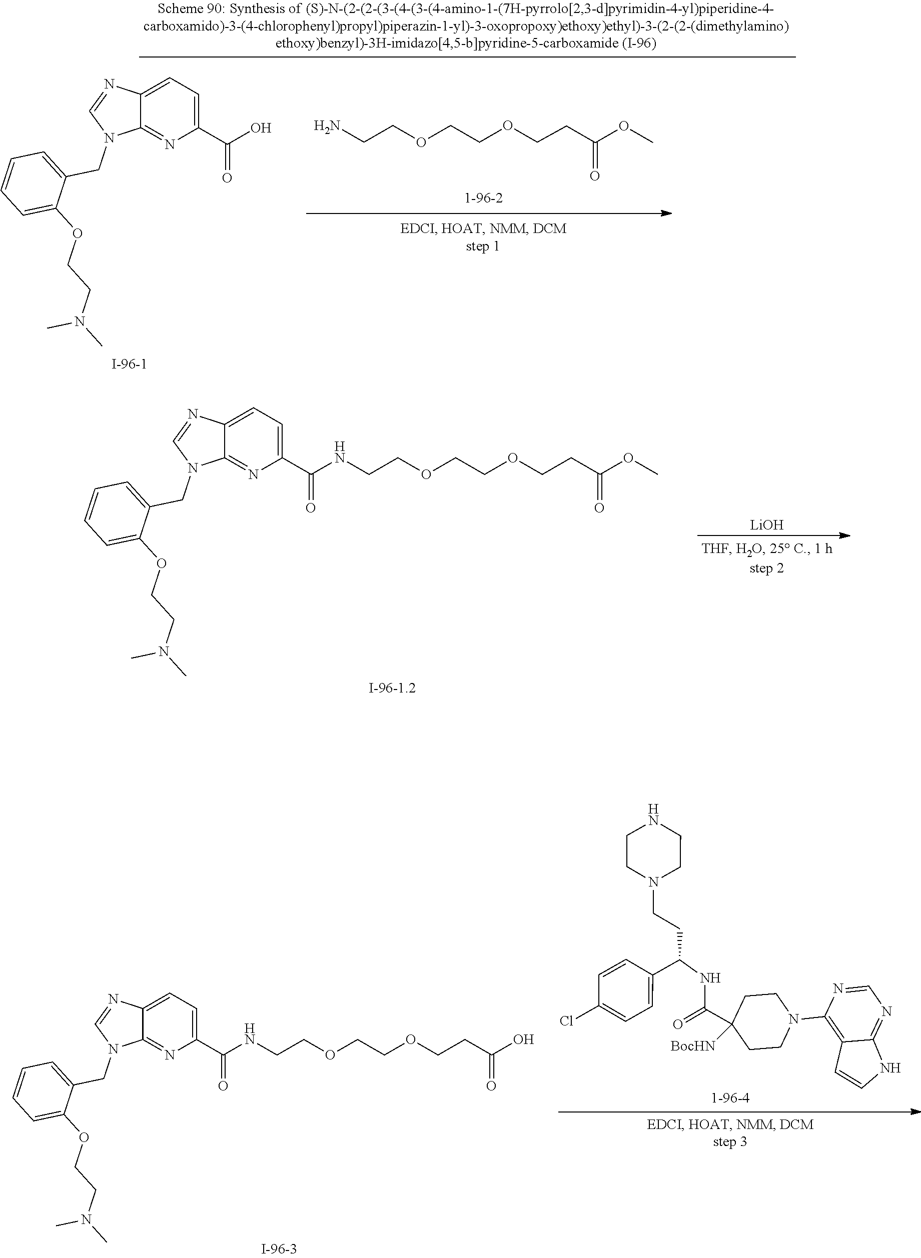 [SMILES: CN(C)CCOc1ccccc1Cn1cnc2ccc(C(=O)O)nc21 ; COC(=O)CCOCCOCCN ; COC(=O)CCOCCOCCNC(=O)c1ccc2ncn(Cc3ccccc3OCCN(C)C)c2n1 ; "LiOH" ; CN(C)CCOc1ccccc1Cn1cnc2ccc(C(=O)NCCOCCOCCC(=O)O)nc21 ; CC(C)(C)OC(=O)NC1(C(=O)N[C@@H](CCN2CCNCC2)c2ccc(Cl)cc2)CCN(c2ncnc3[nH]ccc23)CC1]

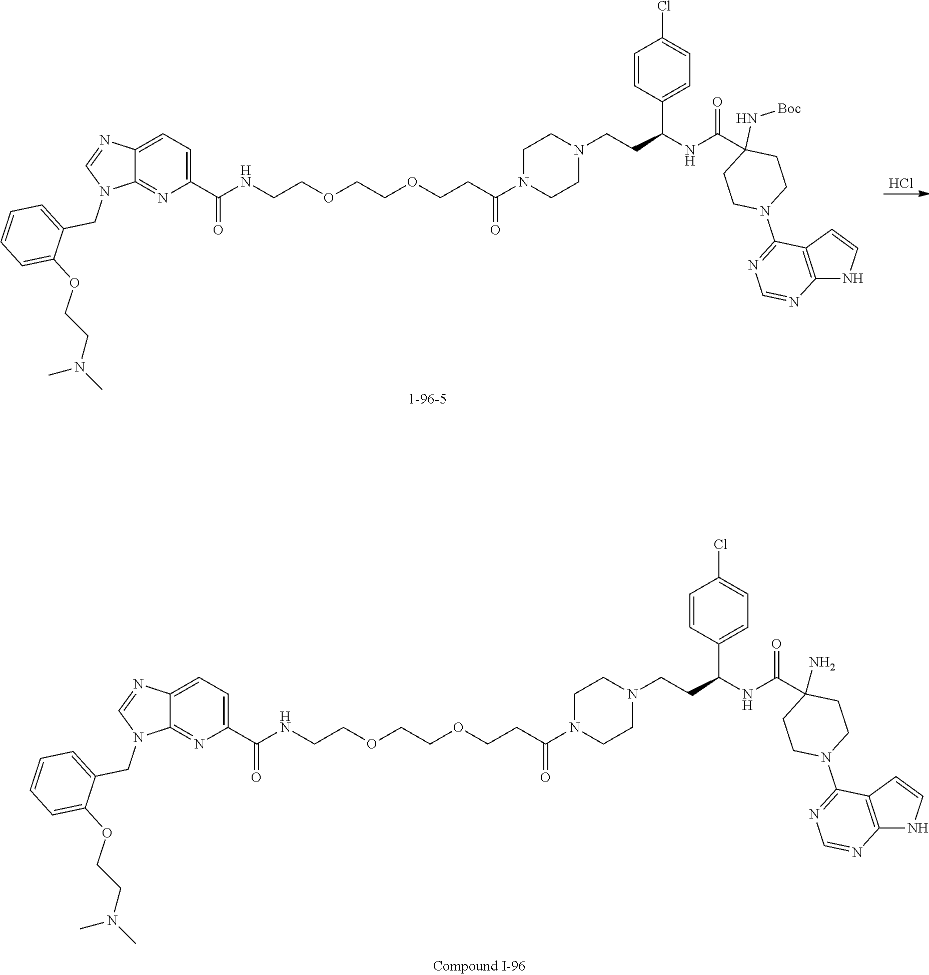 [SMILES: CN(C)CCOc1ccccc1Cn1cnc2ccc(C(=O)NCCOCCOCCC(=O)N3CCN(CC[C@H](NC(=O)C4(NC(=O)OC(C)(C)C)CCN(c5ncnc6[nH]ccc56)CC4)c4ccc(Cl)cc4)CC3)nc21 ; Cl ; CN(C)CCOc1ccccc1Cn1cnc2ccc(C(=O)NCCOCCOCCC(=O)N3CCN(CC[C@H](NC(=O)C4(N)CCN(c5ncnc6[nH]ccc56)CC4)c4ccc(Cl)cc4)CC3)nc21]

### Step 1: Synthesis of I-96-1.2

A mixture of I-96-1 (0.1 g, 293.79 μmol, 1 eq), I-96-2 (80.27 mg, 352.55 μmol, 1.2 eq, HCl) and HOAt (39.99 mg, 293.79 μmol, 41.10 μL, 1 eq) in DCM (1 mL) was added EDCI (112.64 mg, 587.59 μmol, 2 eq) and NMM (148.59 mg, 1.47 mmol, 161.51 μL, 5 eq). The mixture was stirred at 25° C. for 12 hrs. The mixture was concentrated under vacuum. The residue was combined with another batch and to purification. The residue was purified by Pre-HPLC (column: Waters Xbridge 150*25 mm*5 um; mobile phase: [water (NH4HCO3)-ACN]; gradient: 60%-90% B over 9 min) and concentrated under vacuum to get I-96-1.2 (123.9 mg, 232.57 μmol, 79.16% yield, 96.404% purity) as yellow oil. LCMS (Method G): R^{t}=0.526 min, [M+H]⁺=514.3. ¹H NMR (400 MHZ, DMSO-d₆) δ=8.77-8.68 (m, 2H), 8.18 (d, J=8.4 Hz, 1H), 7.97 (d, J=8.4 Hz, 1H), 7.46-7.43 (m, 1H), 7.29-7.27 (m, 1H), 7.04 (d, J=8.0 Hz, 1H), 6.92-6.90 (m, 1H), 5.52 (s, 2H), 4.08-4.06 (m, 2H), 3.64-3.56 (m, 6H), 3.55 (s, 3H), 3.54-3.49 (m, 4H), 2.61-2.58 (m, 2H), 2.48 (s, 2H), 2.21 (s, 6H).

### Step 2: Synthesis of I-96-3

To a solution of I-96-1.2 (100 mg, 194.71 μmol, 1 eq) in MeOH (0.5 mL) and H₂O (0.5 mL) was added LiOH·H2O (16.34 mg, 389.42 μmol, 2 eq). The mixture was stirred at 25° C. for 1 hr. The mixture was adjusted pH=4 with 2M HCl and concentrated under vacuum. The crude product was purified by reversed-phase HPLC (0.1% FA condition) and concentrated under vacuum to remove MeCN and dried by lyophilization to get I-96-3 (100 mg, crude) as yellow solid. LCMS (Method G): R^{t}=0.315 min, [M+H]⁺=500.1.

### Step 3: Synthesis of I-96-5

A mixture of I-96-3 (100 mg, 200.18 μmol, 1 eq), I-96-4 (119.54 mg, 200.18 μmol, 1 eq) and HOAt (27.25 mg, 200.18 μmol, 28.00 μL, 1 eq) in DMF (2 mL) was added EDCI (76.75 mg, 400.35 μmol, 2 eq) and NMM (101.24 mg, 1.00 mmol, 110.04 μL, 5 eq). The mixture was stirred at 25° C. for 12 hrs. The mixture was concentrated under vacuum. The residue was purified by Pre-HPLC (column: Phenomenex luna C18 150*40 mm*15 um; mobile phase: [water (FA)-ACN]; gradient: 2%-32% B over 15 min) and concentrated under vacuum to get I-96-5 (70 mg, 64.89 μmol, 32.42% yield) as yellow oil. LCMS (Method E): R^{t}=0.403 min, [M+H]⁺=1078.9.

### Step 4: Synthesis of I-96

A mixture of I-96-5 (110 mg, 101.98 μmol, 1 eq) in DCM (2 mL) was added HCl/dioxane (4 M, 1.26 mL, 49.31 eq). The mixture was stirred at 25° C. for 0.5 hr. The mixture was concentrated under vacuum to get I-96 (98.1 mg, 93.03 μmol, 91.23% yield, 96.256% purity, HCl) as yellow solid. LCMS (Method G): R^{t}=0.576 min, [M+H]⁺=978.7. SFC: R^{t}=1.914 min. ¹H NMR (400 MHZ, DMSO-d₆) δ=12.89 (br s, 1H), 11.53-11.32 (m, 1H), 10.99 (br d, J=10.0 Hz, 1H), 9.77-9.58 (m, 1H), 9.11 (br s, 1H), 9.02 (s, 1H), 8.81-8.80 (m, 1H), 8.40 (s, 1H), 8.27 (d, J=8.4 Hz, 1H), 8.03 (d, J=8.4 Hz, 1H), 7.51-7.46 (m, 2H), 7.44 (br s, 1H), 7.42-7.38 (m, 2H), 7.36-7.30 (m, 1H), 7.09 (d, J=8.0 Hz, 1H), 7.02 (br d, J=1.6 Hz, 1H), 6.96-6.95 (m, 1H), 5.77 (s, 2H), 4.95 (br s, 1H), 4.42-4.39 (m, 2H), 4.35-4.29 (m, 2H), 4.23-4.13 (m, 6H), 3.62-3.58 (m, 4H), 3.56 (s, 2H), 3.54-3.52 (m, 4H), 3.49-3.37 (m, 4H), 3.22-3.21 (m, 1H), 3.13-2.99 (m, 3H), 2.94-2.87 (m, 1H), 2.84 (d, J=4.8 Hz, 6H), 2.67-2.62 (m, 1H), 2.60-2.54 (m, 3H), 2.39-2.30 (m, 1H), 2.24-2.11 (m, 2H), 2.05-1.95 (m, 1H).

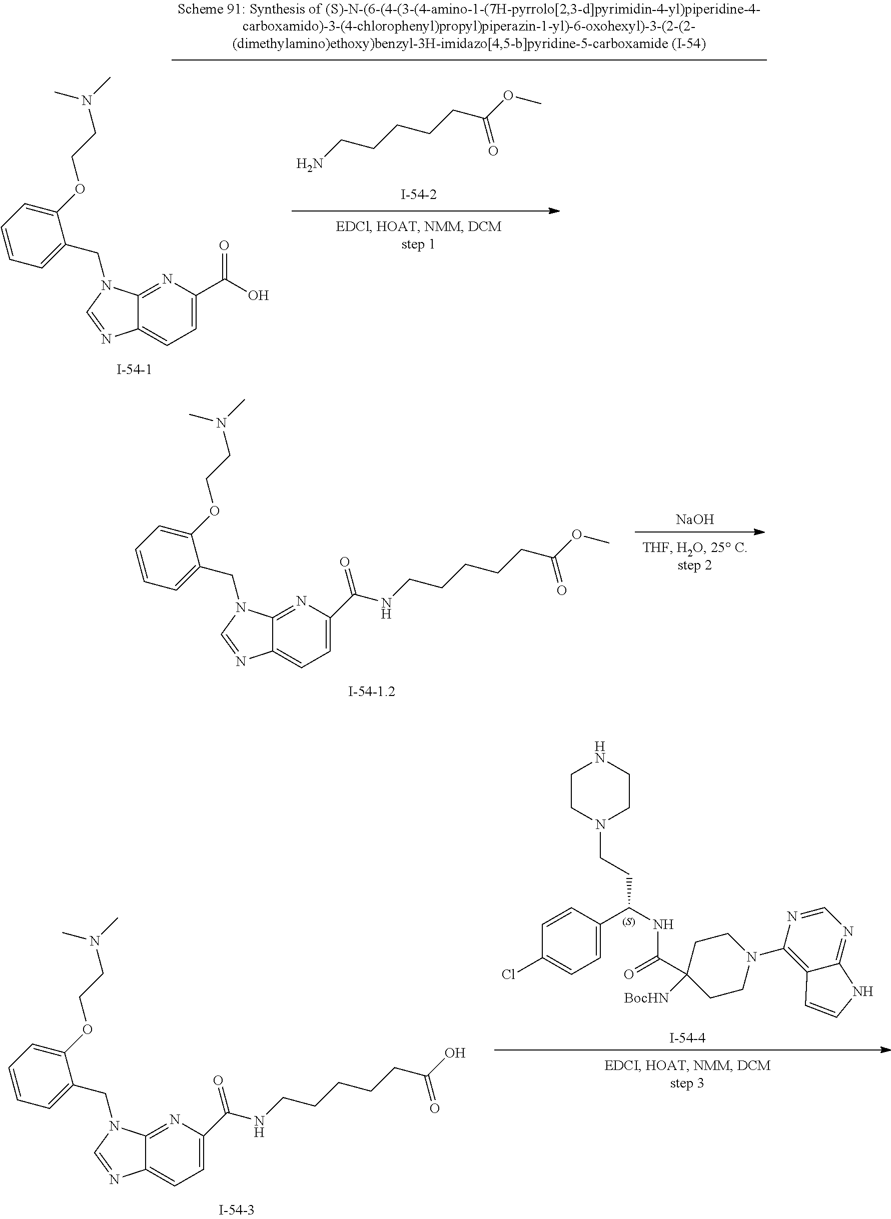

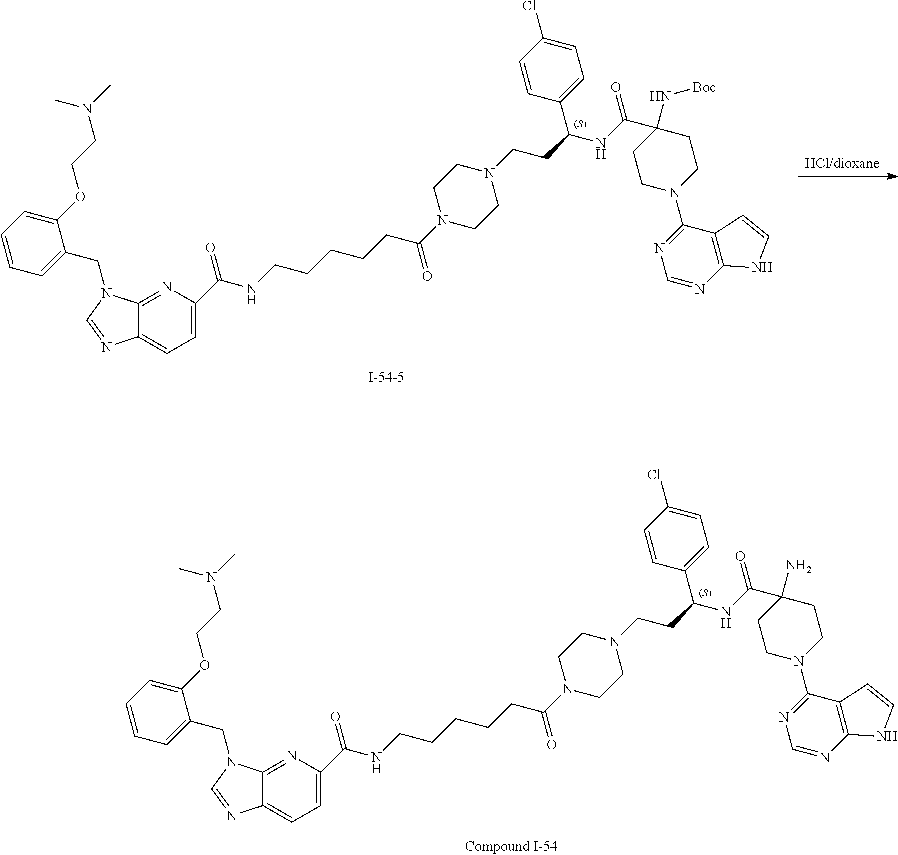

### Step 1: Synthesis of I-54-1.2

To a solution of I-54-1 (900 mg, 2.64 mmol, 1 eq, sodium salt) in DCM (2 mL) was added HOAt (179.95 mg, 1.32 mmol, 0.5 eq), I-54-2 (460.71 mg, 3.17 mmol, 1.2 eq), NMM (1.34 g, 13.22 mmol, 1.45 mL, 5 eq) and EDCI (1.01 g, 5.29 mmol, 2 eq). The mixture was stirred at 25° C. for 12 hr. The mixture was concentrated under vacuum to give a residue. The residue was purified by Pre-HPLC (column: Phenomenex luna C18 150*40 mm*15 um; mobile phase: [water (FA)-ACN]; gradient: 8%-38% B over 15 min) to get I-54-1.2 (930 mg, 1.97 mmol, 74.49% yield, 99.017% purity) as yellow oil, LCMS (Method G): R^{t}=0.578 min, [M+H]⁺=468.2. ¹H NMR (400 MHZ, DMSO-d₆) δ=8.80-8.67 (m, 2H), 8.21-8.13 (m, 2H), 8.01-7.92 (m, 1H), 7.49 (br d, J=7.3 Hz, 1H), 7.29 (br t, J=7.6 Hz, 1H), 7.04 (br d, J=8.1 Hz, 1H), 6.91 (t, J=7.4 Hz, 1H), 5.56 (s, 2H), 4.13 (br t, J=5.4 Hz, 2H), 3.58-3.55 (m, 3H), 3.35 (q, J=6.6 Hz, 2H), 2.78 (br s, 2H), 2.36-2.24 (m, 8H), 1.62-1.50 (m, 4H), 1.38-1.32 (m, 2H).

### Step 2: Synthesis of I-54-3

To a solution of I-54-1.2 (150 mg, 320.81 μmol, 1 eq) in THF (1 mL) and MeOH (1 mL) was added NaOH (1 M, 962.44 μL, 3 eq). The mixture was stirred at 25° C. for 1 h. The mixture was concentrated under vacuum to remove THE/MeOH, the aqueous was adjusted to pH=3 by 1N HCl and then purified by reversed-phase HPLC (0.1% FA condition) to give I-54-3 (120 mg, 264.59 μmol, 82.47% yield) as a white solid. LCMS (Method E): R^{t}=0.388 min, [M+H]⁺=454.2.

### Step 3: Synthesis of I-54-5

To a solution of I-54-3 (100 mg, 220.49 μmol, 1 eq) in DMF (1 mL) was added HOAt (15.01 mg, 110.24 μmol, 0.5 eq), I-54-4 (131.67 mg, 220.49 μmol, 1 eq), NMM (111.51 mg, 1.10 mmol, 121.21 μL, 5 eq) and EDCI (84.54 mg, 440.98 μmol, 2 eq). The mixture was stirred at 25° C. for 1 hr. To the reaction mixture was added 1 ml of MeOH and filtered. The filtrate was purified by Prep-HPLC (column: Waters Xbridge 150*25 mm*5 um; mobile phase: [water (NH4HCO3)-ACN]; gradient: 38%-68% B over 9 min) to give I-54-5 (100 mg, 96.84 μmol, 43.92% yield) as a white solid. LCMS (Method G): R^{t}=0.707 min, [M+H]⁺=1032.7.

### Step 4: Synthesis of I-54

To a solution of I-54-5 (100 mg, 96.84 μmol, 1 eq) in dioxane (1 mL) was added HCl/dioxane (4 M, 1 mL, 41.31 eq). The mixture was stirred at 25° C. for 1 h. The mixture was concentrated under vacuum to remove DCM to give a yellow oil. The yellow oil was diluted with MeOH (0.5 ml) and H₂O (20 ml) to give I-54 (100.88 mg, crude, HCl) as a white solid. LCMS (Method G): R^{t}=0.633 min, [M+H]⁺=932.6. SFC: R^{t}=0.924 min. ¹H NMR (400 MHz, DMSO-d₆) δ=13.00 (br s, 1H), 11.57 (br d, J=2.0 Hz, 1H), 11.18 (br s, 1H), 9.78 (br d, J=7.2 Hz, 1H), 9.32 (s, 1H), 9.24 (br s, 2H), 8.96 (m, 1H), 8.46 (s, 1H), 8.37 (d, J=8.4 Hz, 1H), 8.14 (d, J=8.4 Hz, 1H), 7.60-7.45 (m, 6H), 7.41 (m, 1H), 7.15 (m, 1H), 7.09 (br s, 1H), 7.02 (m, 1H), 5.90 (s, 2H), 4.50-4.44 (m, 3H), 4.37-4.24 (m, 4H), 4.10 (br d, J=12.6 Hz, 1H), 3.64-3.40 (m, 8H), 3.30 (br d, J=2.8 Hz, 1H), 3.22-3.08 (m, 3H), 3.03-2.95 (m, 1H), 2.90 (d, J=4.8 Hz, 6H), 2.75-2.64 (m, 2H), 2.42 (m, 3H), 2.30-2.21 (m, 2H), 2.12-2.04 (m, 1H), 1.69-1.56 (m, 4H), 1.48-1.38 (m, 2H).

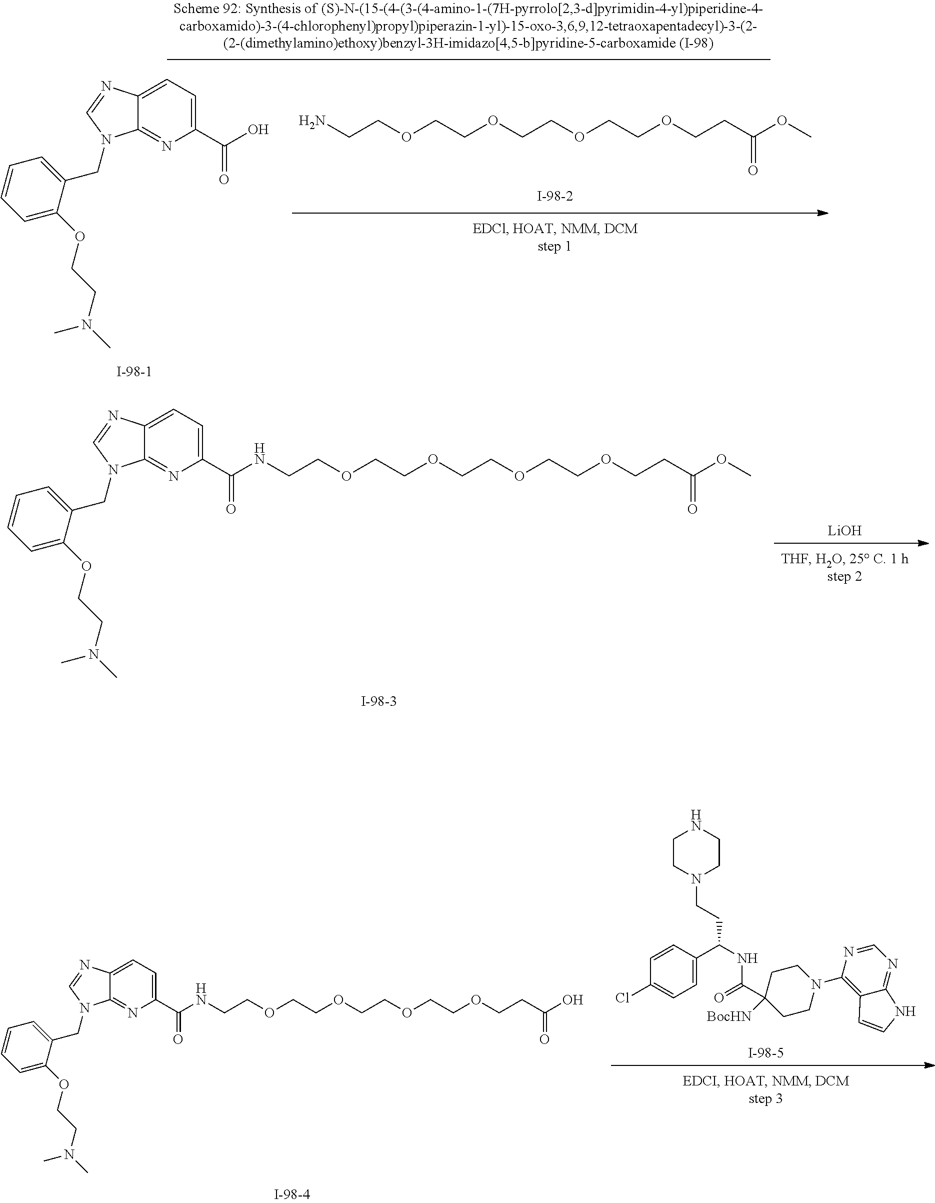

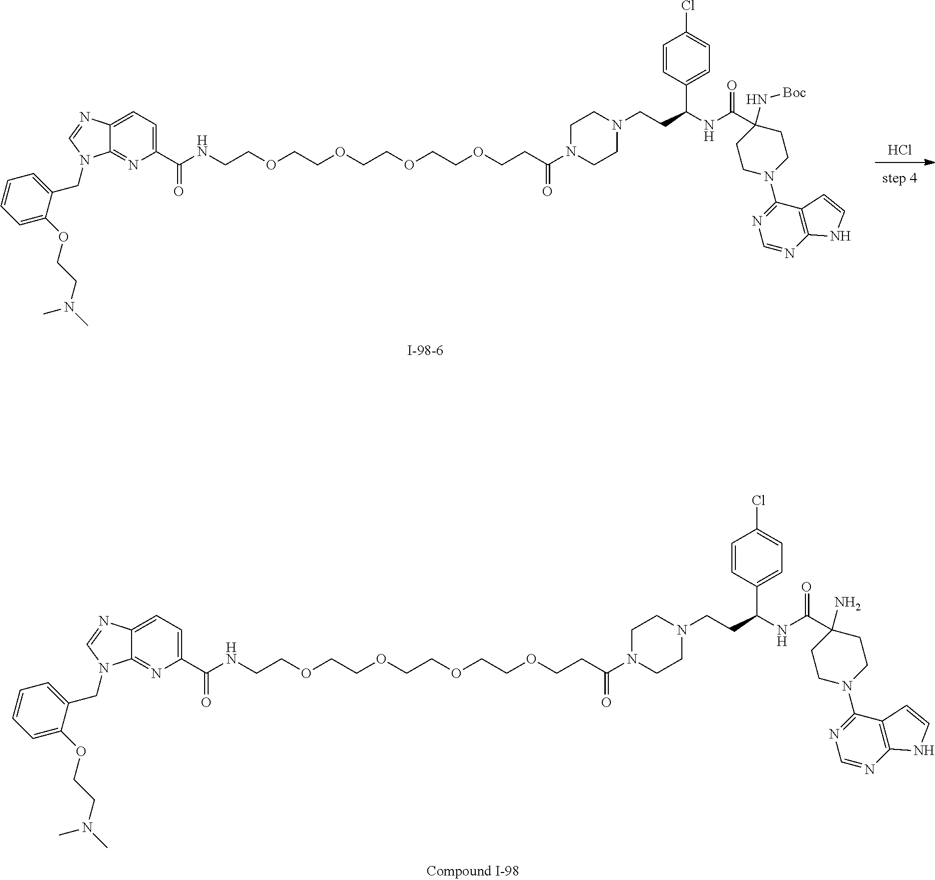 [SMILES: CN(C)CCOc1ccccc1Cn1cnc2ccc(C(=O)NCCOCCOCCOCCOCCC(=O)N3CCN(CC[C@H](NC(=O)C4(NC(=O)OC(C)(C)C)CCN(c5ncnc6[nH]ccc56)CC4)c4ccc(Cl)cc4)CC3)nc21 ; Cl ; CN(C)CCOc1ccccc1Cn1cnc2ccc(C(=O)NCCOCCOCCOCCOCCC(=O)N3CCN(CC[C@H](NC(=O)C4(N)CCN(c5ncnc6[nH]ccc56)CC4)c4ccc(Cl)cc4)CC3)nc21]

### Step 1: Synthesis of I-98-3

A mixture of I-98-1 (0.1 g, 293.79 μmol, 1 eq), I-98-2 (111.33 mg, 352.55 μmol, 1.2 eq, HCl) and HOAt (39.99 mg, 293.79 μmol, 41.10 μL, 1 eq) in DCM (1 mL) was added EDCI (112.64 mg, 587.59 μmol, 2 eq) and NMM (148.59 mg, 1.47 mmol, 161.51 μL, 5 eq). The mixture was stirred at 25° C. for 12 hrs. LCMS showed reactant 1 was consumed and one peak of desired MW was detected. The mixture was concentrated under vacuum. The residue was purified by Pre-HPLC (column: Waters Xbridge 150*25 mm*5 um; mobile phase: [water (NH4HCO3)-ACN]; gradient: 60%-90% B over 9 min) and concentrated under vacuum to get I-98-3 (120 mg, 199.44 μmol, 67.88% yield) as yellow oil. LCMS (Method G): R^{t}=0.528 min, [M+H]⁺=602.2. 1H NMR (400 MHZ, DMSO-d₆) δ=8.79-8.63 (m, 2H), 8.25-8.14 (m, 1H), 8.05-7.91 (m, 1H), 7.50-7.41 (m, 1H), 7.33-7.25 (m, 1H), 7.10-7.01 (m, 1H), 6.90 (q, J=6.9 Hz, 1H), 5.50 (d, J=16.4 Hz, 2H), 4.12-4.03 (m, 2H), 3.91 (s, 1H), 3.62-3.56 (m, 7H), 3.56-3.47 (m, 11H), 3.46-3.42 (m, 8H), 2.60 (q, J=5.5 Hz, 2H), 2.25-2.17 (m, 6H), 1H NMR (400 MHZ, DMSO-d₆) δ=8.80-8.67 (m, 2H), 8.23-8.11 (m, 2H), 7.97 (d, J=8.4 Hz, 1H), 7.46-7.44 (m, 1H), 7.33-7.25 (m, 1H), 7.04 (d, J=8.0 Hz, 1H), 6.92-6.91 (m, 1H), 5.75 (s, 1H), 5.52 (s, 2H), 4.10-4.07 (m, 2H), 3.63-3.58 (m, 5H), 3.57 (s, 4H), 3.56-3.52 (m, 4H), 3.51-3.47 (m, 3H), 3.46-3.44 (m, 2H), 3.43 (s, 3H), 2.65-2.62 (m, 2H), 2.24 (s, 6H).

### Step 2: Synthesis of I-98-4

To a solution of I-98-3 (100 mg, 166.20 μmol, 1 eq) in MeOH (0.5 mL) and H₂O (0.5 mL) was added LiOH·H2O (13.95 mg, 332.40 μmol, 2 eq). The mixture was stirred at 25° C. for 1 hr. The mixture was adjusted to pH=4 with 2M HCl and concentrated under vacuum. The crude product was purified by reversed-phase HPLC (0.1% FA condition) and concentrated under vacuum to remove MeCN and dried by lyophilization to get I-98-4 (95 mg, 161.66 μmol, 97.27% yield) as yellow solid. LCMS (Method G): R^{t}=0.322 min, [M+H]⁺=588.2.

### Step 3: Synthesis of I-98-6

A mixture of I-98-4 (95 mg, 161.66 μmol, 1 eq), I-98-5 (96.53 mg, 161.66 μmol, 1 eq) and HOAt (22.00 mg, 161.66 μmol, 22.61 μL, 1 eq) in DMF (2 mL) was added EDCI (61.98 mg, 323.31 μmol, 2 eq) and NMM (81.76 mg, 808.29 μmol, 88.87 μL, 5 eq). The mixture was stirred at 25° C. for 12 hrs. The mixture was concentrated under vacuum. The residue was purified by Pre-HPLC (column: Phenomenex luna C18 150*40 mm*15 um; mobile phase: [water (FA)-ACN]; gradient: 2%-32% B over 15 min) and concentrated under vacuum to get I-98-6 (30 mg, 25.71 μmol, 15.90% yield) as yellow oil. LCMS (Method E): R^{t}=0.410 min, [M+H]⁺=1167.0.

### Step 4: Synthesis of I-98

A mixture of I-98-6 (75 mg, 64.28 μmol, 1 eq) in DCM (1 mL) was added HCl/dioxane (4 M, 1.25 mL, 77.79 eq). The mixture was stirred at 25° C. for 0.5 hr. The mixture was concentrated under vacuum. This reaction used in next step without purification to get I-98 (68.78 mg, 58.87 μmol, 91.58% yield, 94.418% purity, HCl) as yellow solid. LCMS (Method G): R^{t}=0.581 min, [M+H]⁺=1066.7. SFC: R^{t}=1.913 min. 1H NMR (400 MHZ, DMSO-d₆) δ=12.87 (br s, 1H), 11.41 (br s, 1H), 10.95 (br s, 1H), 9.64 (br d, J=6.4 Hz, 1H), 9.10 (br s, 1H), 8.98 (s, 1H), 8.79-8.78 (m, 1H), 8.40 (s, 1H), 8.26 (d, J=8.4 Hz, 1H), 8.03 (d, J=8.4 Hz, 1H), 7.49 (br d, J=2.8 Hz, 1H), 7.47-7.43 (m, 3H), 7.42-7.38 (m, 2H), 7.36-7.30 (m, 1H), 7.09 (d, J=8.0 Hz, 1H), 7.04-7.00 (m, 1H), 6.96-6.95 (m, 1H), 5.76 (s, 2H), 4.95 (br d, J=3.2 Hz, 1H), 4.42-4.39 (m, 2H), 4.38-4.21 (m, 4H), 4.20-4.11 (m, 3H), 4.08-4.02 (m, 2H), 3.60 (br d, J=5.2 Hz, 2H), 3.58-3.57 (m, 2H), 3.55-3.53 (m, 4H), 3.48 (br d, J=2.4 Hz, 2H), 3.46 (br d, J=7.2 Hz, 2H), 3.44 (s, 6H), 3.14-2.97 (m, 4H), 2.93-2.87 (m, 1H), 2.84 (d, J=4.8 Hz, 6H), 2.59 (br d, J=2.8 Hz, 5H), 2.37-2.30 (m, 1H), 2.26-2.10 (m, 3H), 2.02-1.94 (m, 1H).

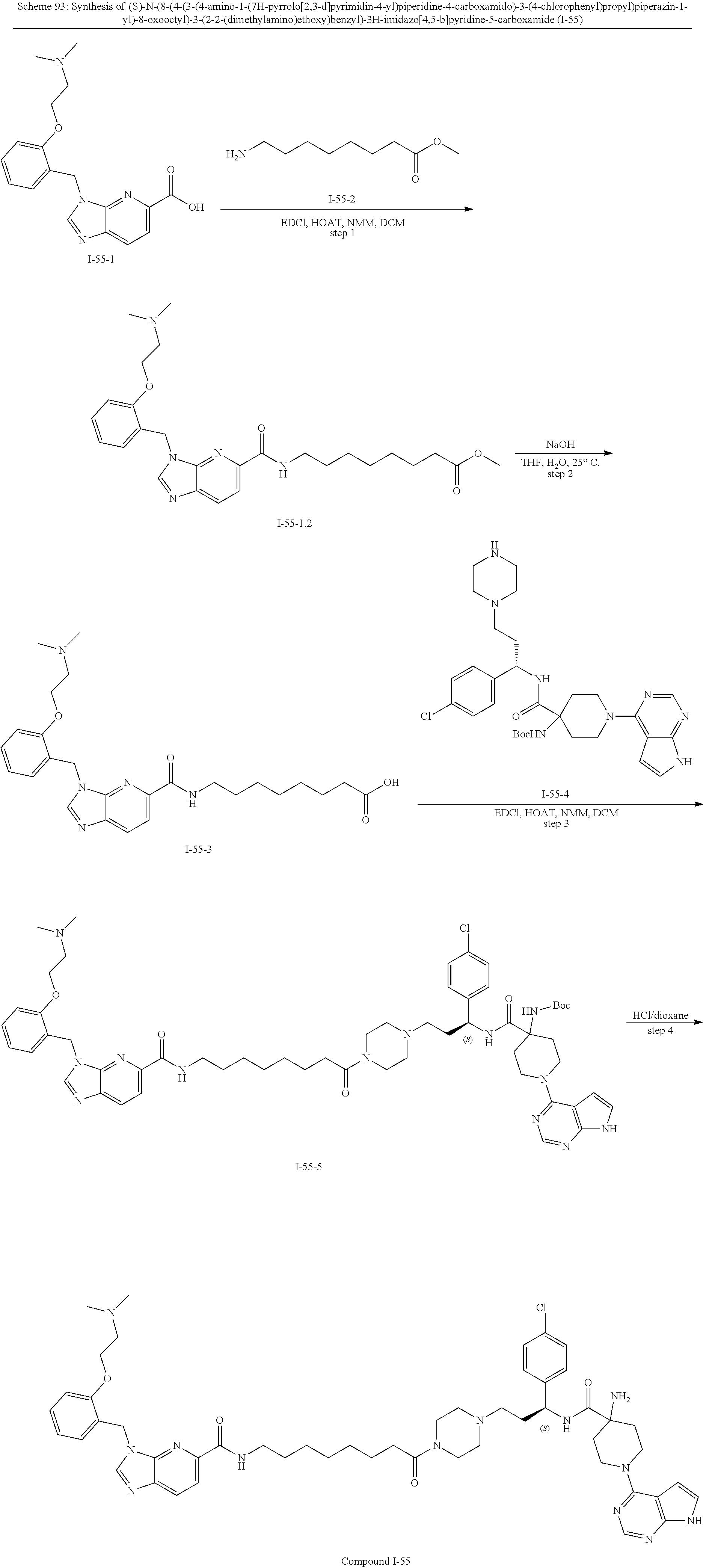

### Step 1: Synthesis of I-55-1.2

To a solution of I-55-1 (900 mg, 2.64 mmol, 1 eq) in DCM (18 mL) was added HOAt (179.95 mg, 1.32 mmol, 0.5 eq), I-55-2 (549.72 mg, 3.17 mmol, 1.2 eq), NMM (1.34 g, 13.22 mmol, 1.45 mL, 5 eq) and EDCI (1.01 g, 5.29 mmol, 2 eq). The mixture was stirred at 25° C. for 24 hr. The mixture was concentrated under vacuum to give a residue. The residue was purified by Pre-HPLC (column: Waters Xbridge 150*25 mm*5 um; mobile phase: [water (ammonia hydroxide v/v)-ACN]; gradient: 38%-68% B over min) to get I-55-1.2 (1.1 g, 1.88 mmol, 71.21% yield, 84.835% purity) was obtained as yellow oil, LCMS (Method G): R^{t}=0.642 min, [M+H]⁺=496.4. ¹H NMR (400 MHZ, DMSO-d₆) δ=8.73 (s, 2H), 8.16 (d, J=8.3 Hz, 1H), 7.98-7.92 (m, 1H), 7.53-7.46 (m, 1H), 7.34-7.25 (m, 1H), 7.03 (d, J=8.2 Hz, 1H), 6.89 (t, J=7.4 Hz, 1H), 5.58-5.51 (m, 1H), 5.53 (s, 1H), 4.07 (t, J=5.7 Hz, 2H), 3.56 (s, 3H), 2.59 (t, J=5.7 Hz, 2H), 2.28 (t, J=7.3 Hz, 2H), 2.21 (s, 6H), 1.54 (td, J=7.2, 14.9 Hz, 4H), 1.33-1.25 (m, 6H).

### Step 2: Synthesis of I-55-3

To a solution of I-55-1.2 (120 mg, 203.38 μmol, 1 eq) in THF (1 mL) and MeOH (1 mL) was added

NaOH (1 M, 610.15 μL, 3 eq). The mixture was stirred at 25° C. for 1 h. The mixture was concentrated under vacuum to remove THF/MeOH and adjusted to pH=7 by 1 N HCl (0.61 mL). The aqueous was purified by reversed-phase HPLC (0.1% FA condition) to give I-55-3 (80 mg, 166.12 μmol, 81.68% yield) as a white solid. LCMS (Method E): R^{t}=0.413 min, [M+H]⁺=482.2.

### Step 3: Synthesis of I-55-5

To a solution of I-55-3 (80 mg, 166.12 μmol, 1 eq) in DMF (2 mL) was added HOAt (11.31 mg, 83.06 μmol, 0.5 eq), I-55-4 (99.20 mg, 166.12 μmol, 1 eq), NMM (84.01 mg, 830.59 μmol, 91.32μ, 5 eq) and EDCI (63.69 mg, 332.24 μmol, 2 eq). The mixture was stirred at 25° C. for 1 hr. To the reaction mixture was added 1 ml of MeOH and filtered. The filter was purified by Prep-HPLC (column: Waters Xbridge 150*25 mm*5 um; mobile phase: [water (NH4HCO3)-ACN]; gradient: 40%-70% B over 9 min) to give I-55-5 (80 mg, 75.17 μmol, 45.25% yield, 99.669% purity) as a white solid. LCMS (Method G): R^{t}=0.731 min, [M+H]⁺=1060.7.

### Step 4: Synthesis of I-55

To a solution of I-55-5 (80 mg, 75.42 μmol, 1 eq) in dioxane (0.6 mL) was added HCl/dioxane (4 M, 0.6 mL, 31.82 eq). The mixture was stirred at 25° C. for 1 h. The mixture was concentrated under vacuum to remove DCM to give a yellow oil. The yellow oil was diluted with MeOH (0.5 ml) and H₂O (20 ml) to give I-55 (60.18 mg, 60.36 μmol, 80.03% yield, 100% purity, HCl) as a white solid. LCMS (Method G): R^{t}=0.668 min, [M+H]⁺=960.6. SFC: R^{t}=1.075 min. ¹H NMR (400 MHz, DMSO-d₆) δ=13.01 (br s, 1H), 11.57 (m, 1H), 11.20 (br s, 1H), 9.78 (m, 1H), 9.40 (s, 1H), 9.25 (br s, 2H), 8.95 (m, 1H), 8.46 (s, 1H), 8.38 (d, J=8.4 Hz, 1H), 8.15 (d, J=8.4 Hz, 1H), 7.63 (d, J=7.2 Hz, 1H), 7.58-7.41 (m, 6H), 7.15 (d, J=8.4 Hz, 1H), 7.11-7.08 (m, 1H), 7.02 (m, 1H), 5.91 (s, 2H), 4.43 (br s, 8H), 4.12-4.05 (m, 1H), 3.65-3.58 (m, 3H), 3.53 (m, 1H), 3.42 (m, 2H), 3.35-3.26 (m, 1H), 3.21-3.07 (m, 3H), 3.03-2.94 (m, 1H), 2.90 (d, J=4.8 Hz, 6H), 2.75-2.63 (m, 2H), 2.39 (m, 3H), 2.30-2.21 (m, 2H), 2.08 (m, 1H), 1.64 (m, 2H), 1.58-1.50 (m, 2H), 1.39 (br s, 6H).

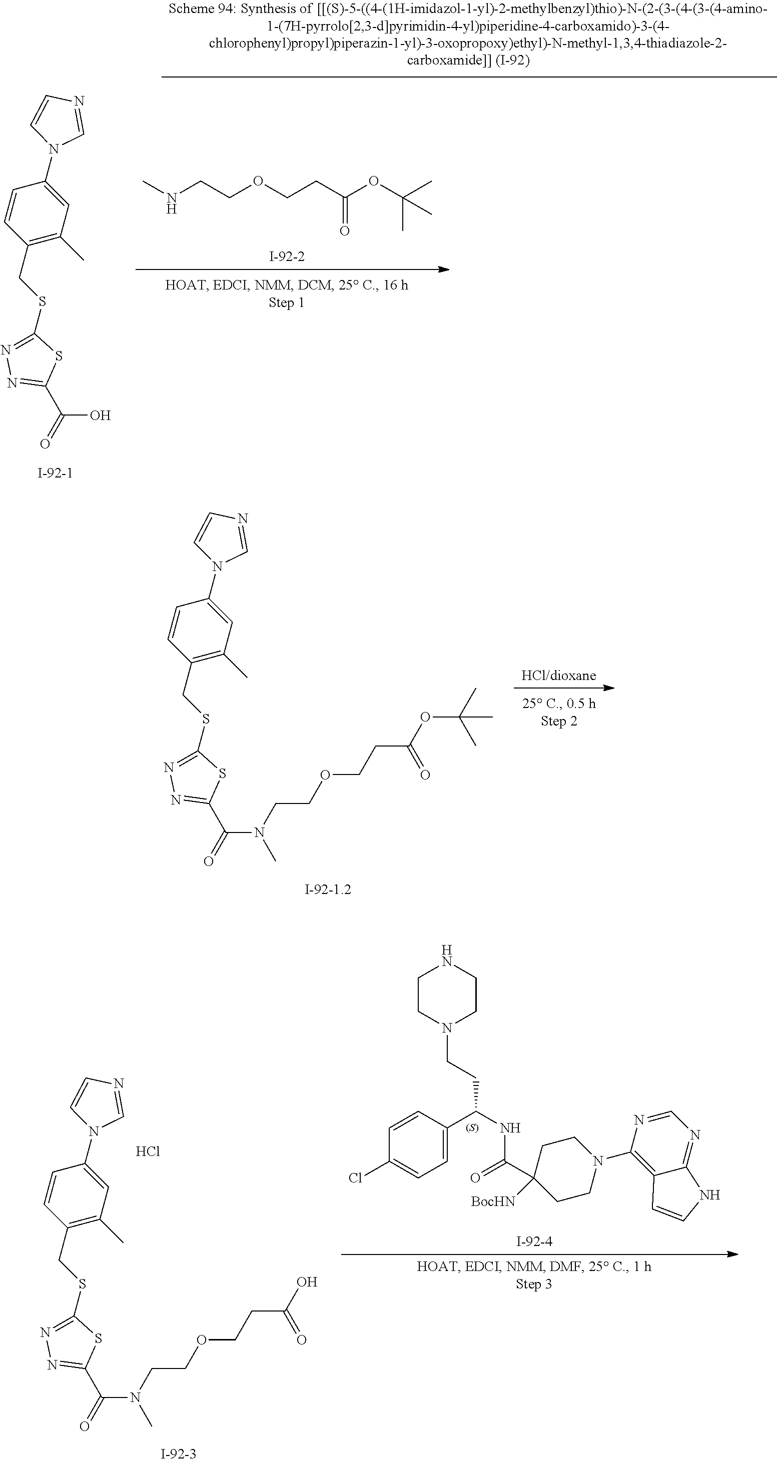

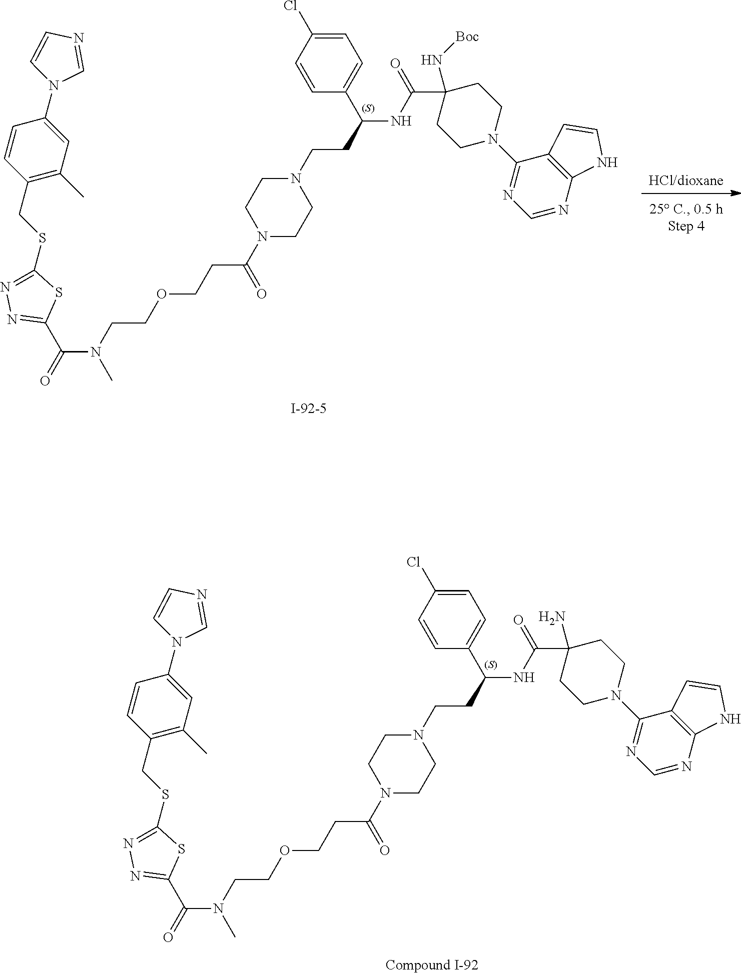

### Step 1: Synthesis of I-92-1.2

To a solution of I-92-1 (1.1 g. 3.10 mmol, 1 eq. sodium salt) and I-92-2 (692.10 mg, 3.40 mmol. 1.1 eq). EDCI (1.78 g. 9.29 mmol, 3 eq) and HOAt (631.93 mg, 4.64 mmol, 1.5 eq) in DCM (10 mL) was added NMM (1.57 g. 15.48 mmol, 1.70 mL, 5 eq) and then the mixture was stirred at 25° C. for 16 hr. The reaction mixture was quenched by H₂O (10 mL), extracted with DCM (5 mL*3). The combined organic layers were dried over Na₂SO₄, filtered and concentrated under reduced pressure to give a residue. The crude residue was purified by reversed phase preparative HPLC (105 g) of SepaFlash® Spherical C18, 20-45 μm, 100 Å; mobile phase: A for H2O+0.1% NH3·H2O and B for acetonitrile; gradient: B 40-50% in 30 min, flow rate: 60 mL/min; column temperature: RT, wavelength: 220 nm/254 nm), after purification, then concentrated to remove organic solvents then lyophilized to give I-92-1.2 (1 g, 1.93 mmol, 62.41% yield, 100% purity) as colorless oil. LCMS (Method B): R^{t}=0.612 min, [M+H]′=518.2. ¹H NMR (400 MHz, DMSO-d₆) δ=8.24 (s, 1H), 7.73 (s, 1H), 7.60-7.52 (m, 2H), 7.50-7.42 (m, 1H), 7.09 (s, 1H), 4.69 (s, 2H), 4.00 (t, J=5.2 Hz, 1H), 3.66-3.50 (m, 5H), 3.42 (s, 1H), 3.05 (s, 2H), 2.46 (s, 3H), 2.41 (t, J=6.0 Hz, 1H), 2.34 (t, J=6.0 Hz, 1H), 1.38-1.33 (m, 9H).

### Step 2: Synthesis of I-92-3

To a solution of I-92-1.2 (0.15 g, 289.76 μmol, 1 eq) in HCl/dioxane (1.5 mL) and then the mixture was stirred at 25° C. for 0.5 hr. The reaction mixture was concentrated under room temperature reduced pressure to remove solvent to give the crude. The crude product was used next step directly without any purification. I-92-3 (0.15 g, crude, HCl) was obtained as yellow oil. LCMS (Method E): R^{t}=0.390 min, [M+H]⁺=462.1. ¹H NMR (400 MHZ, DMSO-d₆) δ=9.67 (s, 1H), 8.29-8.24 (m, 1H), 7.93-7.88 (m, 1H), 7.75-7.67 (m, 2H), 7.64-7.59 (m, 1H), 4.74 (s, 2H), 4.00 (t, J=5.2 Hz, 1H), 3.64-3.58 (m, 6H), 3.05 (s, 2H), 2.49-2.49 (m, 3H), 2.44 (t, J=6.4 Hz, 1H), 2.37 (t, J=6.4 Hz, 1H).

### Step 3: Synthesis of I-92-5

To a solution of I-92-3 (0.1 g, 200.80 μmol, 1 eq, HCl) and I-92-4 (119.91 mg, 200.80 μmol, 1 eq), EDCI (115.48 mg, 602.39 μmol, 3 eq) and NMM (101.55 mg, 1.00 mmol, 110.38 μL, 5 eq) in DMF (1 mL) was added HOAt (27.33 mg, 200.80 μmol, 1 eq) and then the mixture was stirred at 25° C. for 1 hr. The reaction mixture was work up with another batch. The reaction mixture was purified directly without any work up. The reaction mixture was purified by reversed phase preparative HPLC (105 g) of SepaFlash R Spherical C18, 20-45 μm, 100 Å; mobile phase: A for H2O+0.1% NH3·H2O and B for acetonitrile; gradient: B 50-60% in 30 min, flow rate: 60 mL/min; column temperature: RT, wavelength: 220 nm/254 nm), after purification, then concentrated to remove organic solvents then lyophilized to give I-92-5 (0.15 g, 144.13 μmol, 71.78% yield) as an off-white solid. LCMS (Method G): R^{t}=0.643 min, [M+H]⁺=1040.6. SFC: R^{t}=0.742 min. ¹H NMR (400 MHZ, DMSO-d₆) δ=11.66 (br s, 1H), 8.32-8.21 (m, 2H), 8.11 (s, 1H), 7.72 (s, 1H), 7.59-7.50 (m, 2H), 7.45 (d, J=8.0 Hz, 1H), 7.31 (s, 4H), 7.19-7.11 (m, 2H), 7.09 (s, 1H), 6.59 (br s, 1H), 4.93-4.79 (m, 1H), 4.68 (s, 2H), 4.26-4.12 (m, 2H), 4.00 (t, J=5.2 Hz, 1H), 3.66-3.52 (m, 7H), 3.50-3.39 (m, 7H), 3.05 (s, 2H), 2.45 (s, 3H), 2.35-2.19 (m, 6H), 1.96 (br s, 4H), 1.89-1.75 (m, 2H), 1.48-1.30 (m, 9H).

### Step 4: Synthesis of I-92

To a solution of I-92-5 (0.1 g, 96.09 μmol, 1 eq) in dioxane (0.5 mL) was added HCl/dioxane (4 M, 0.5 mL, 20.81 eq) and then the mixture was stirred at 25° C. for 0.5 hr. The reaction mixture was concentrated under reduced pressure (room temperature) to remove solvent to give product. The product was added 30 mL pure water and lyophilized to give I-92 (68.37 mg, 67.88 μmol, 70.64% yield, 97% purity, HCl) as an off-white solid. LCMS (Method G): R^{t}=0.579 min, [M+H]⁺=940.4. SFC: R^{t}=1.067 min. _F2. ¹H NMR (400 MHZ, DMSO-d₆) δ=13.02-12.71 (m, 1H), 11.71-11.36 (m, 1H), 9.90-9.56 (m, 2H), 9.33-8.92 (m, 3H), 8.46-8.24 (m, 2H), 7.93 (s, 1H), 7.76 (br s, 1H), 7.72-7.61 (m, 2H), 7.51-7.36 (m, 5H), 7.01-7.01 (m, 1H), 7.06-6.94 (m, 1H), 4.94 (d, J=2.4 Hz, 1H), 4.74 (s, 2H), 4.43-3.93 (m, 9H), 3.64 (br s, 8H), 3.41 (s, 2H), 3.24-3.09 (m, 3H), 3.04 (s, 2H), 2.96-2.86 (m, 1H), 2.66-2.52 (m, 4H), 2.48 (br s, 3H), 2.39-2.29 (m, 1H), 2.26-2.09 (m, 2H), 1.98-1.97 (m, 1H). ¹H NMR (400 MHZ, DMSO+D2O) δ=9.55 (br s, 1H), 8.34 (s, 1H), 8.15 (s, 1H), 7.80 (s, 1H), 7.70-7.62 (m, 2H), 7.56-7.54 (m, 1H), 7.46-7.30 (m, 5H), 6.93 (br s, 1H), 4.91-4.90 (m, 1H), 4.68 (s, 2H), 4.44-4.25 (m, 3H), 3.94 (br s, 5H), 3.65-3.51 (m, 6H), 3.49-3.39 (m, 2H), 3.34 (s, 1H), 3.20-3.04 (m, 3H), 3.01 (s, 2H), 2.99-2.92 (m, 1H), 2.69-2.51 (m, 4H), 2.47 (s, 3H), 2.35-2.24 (m, 1H), 2.20-2.04 (m, 2H), 1.93-1.91 (m, 1H).

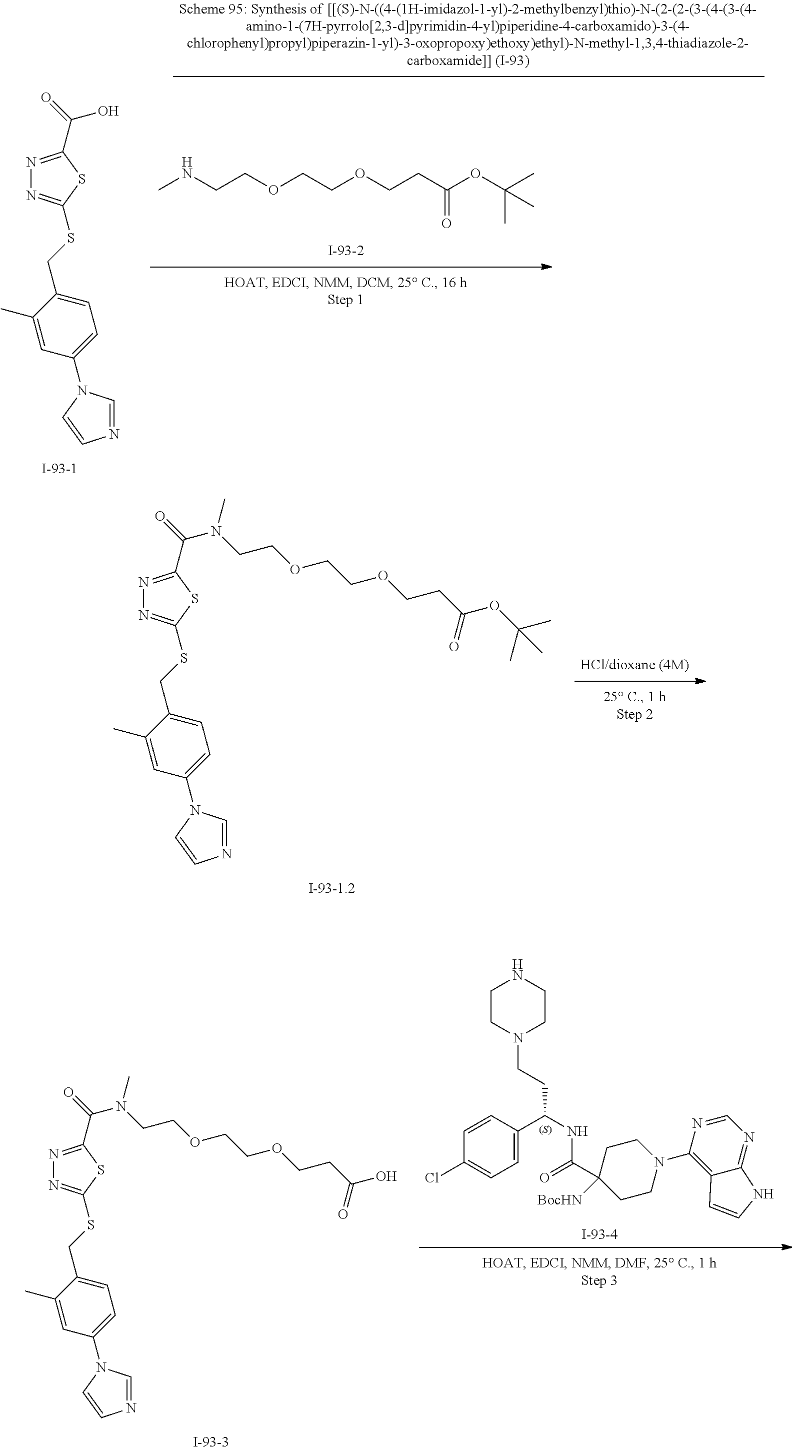

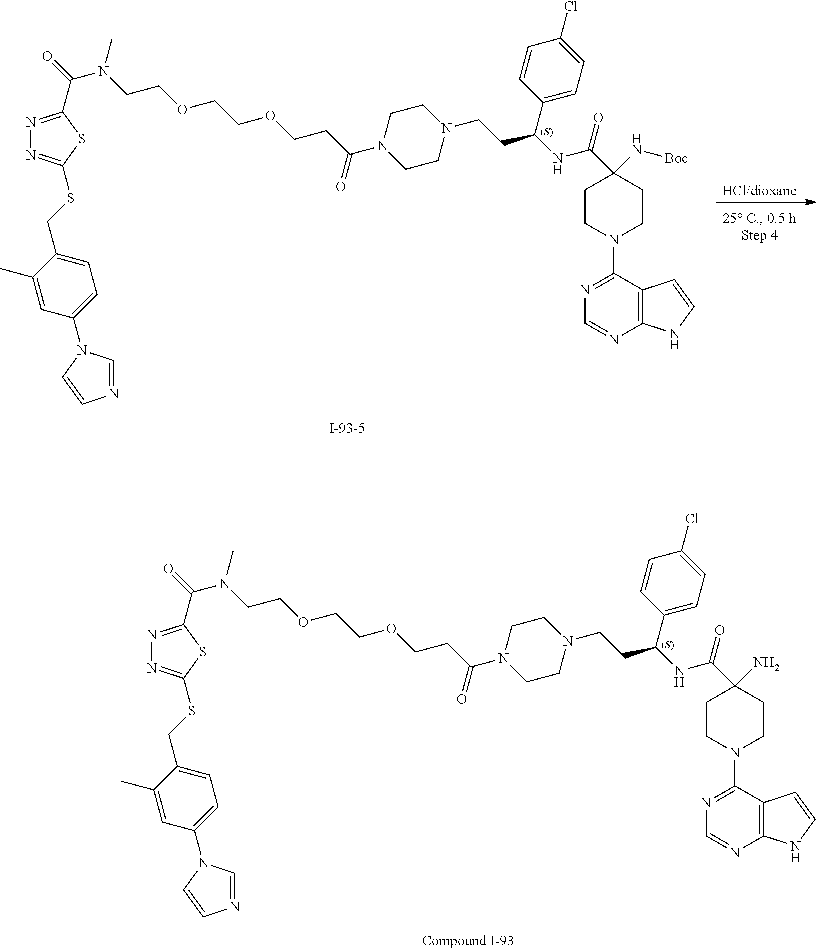

### Step 1: Synthesis of I-93-1.2

To a solution of I-93-1 (1.1 g, 3.10 mmol, 1 eq, Na) and I-93-2 (842.09 mg, 3.40 mmol, 1.1 eq), EDCI (1.78 g, 9.29 mmol, 3 eq) and HOAt (631.93 mg, 4.64 mmol, 1.5 eq) in DCM (10 mL) was added NMM (1.57 g, 15.48 mmol, 1.70 mL, 5 eq) and then the mixture was stirred at 25° C. for 16 hr. The reaction mixture was quenched by H₂O (10 mL), extracted with DCM (5 mL*3). The combined organic layers were dried over Na₂SO₄, filtered and concentrated under reduced pressure to give a residue. The crude residue was purified by reversed phase preparative HPLC (105 g) of SepaFlash® Spherical C18, 20-45 μm, 100 Å; mobile phase: A for H2O+0.1% NH3·H2O and B for acetonitrile; gradient: B 40-50% in 30 min, flow rate: 60 mL/min; column temperature: RT, wavelength: 220 nm/254 nm), after purification, then concentrated to remove organic solvents then lyophilized to give product. I-93-1.2 (1.65 g, 2.94 mmol, 94.90% yield, 100% purity) was obtained as yellow oil. LCMS (Method B): R^{t}=0.614 min, [M+H]⁺=562.3. ¹H NMR (400 MHZ, DMSO-d₆) δ=8.24 (s, 1H), 7.73 (s, 1H), 7.61-7.53 (m, 2H), 7.46-7.44 (m, 1H), 7.09 (s, 1H), 4.69 (s, 2H), 4.01 (t, J=5.2 Hz, 1H), 3.63-3.49 (m, 6H), 3.46-3.37 (m, 4H), 3.06 (s, 2H), 2.46 (s, 3H), 2.41-2.35 (m, 2H), 1.37 (s, 9H),

### Step 2: Synthesis of I-93-3

To a solution of I-93-1.2 (0.15 g, 267.04 μmol, 1 eq) in HCl/dioxane (1.5 mL) and then the mixture was stirred at 25° C. for 1 hr. The reaction mixture was concentrated under room temperature reduced pressure to remove solvent to give I-93-3 (0.15 g, crude, HCl, crude) as yellow oil. LCMS (Method G): R^{t}=0.334 min, [M+H]⁺=506.1. ¹H NMR (400 MHZ, DMSO-d₆) δ=9.69 (s, 1H), 8.28 (t, J=1.6 Hz, 1H), 7.94-7.89 (m, 1H), 7.75-7.60 (m, 3H), 4.74 (s, 2H), 4.01 (t, J=5.2 Hz, 1H), 3.64-3.58 (m, 5H), 3.42 (s, 6H), 3.06 (s, 2H), 2.49-2.48 (m, 3H), 2.41-2.39 (m, 2H).

### Step 3: Synthesis of I-93-5

To a solution of I-93-3 (0.1 g, 184.48 μmol, 1 eq, HCl) and I-93-4 (110.16 mg, 184.48 μmol, 1 eq), EDCI (106.09 mg, 553.43 μmol, 3 eq) and NMM (93.30 mg, 922.39 μmol, 101.41 μL, 5 eq) in DMF (1 mL) was added HOAt (25.11 mg, 184.48 μmol, 1 eq) and then the mixture was stirred at 25° C. for 1 hr. The reaction mixture was purified directly without any work up. The reaction mixture was purified by reversed phase preparative HPLC (105 g) of SepaFlash® Spherical C18, 20-45 μm, 100 Å; mobile phase: A for H2O+0.1% NH₃·H₂O and B for acetonitrile; gradient: B 50-60% in 30 min, flow rate: 60 mL/min; column temperature: RT, wavelength: 220 nm/254 nm), after purification, then concentrated to remove organic solvents then lyophilized to give I-93-5 (0.15 g, 138.28 μmol, 74.96% yield) as an off-white solid. LCMS (Method G): R^{t}=0.649 min, [M+H]⁺=1084.6. SFC: R^{t}=0.734 min. ¹H NMR (400 MHZ, DMSO-d₆) δ=11.65 (br s, 1H), 8.23 (s, 2H), 8.11 (s, 1H), 7.72 (s, 1H), 7.59-7.51 (m, 2H), 7.45-7.43 (m, 1H), 7.31 (s, 4H), 7.16-7.14 (m, 1H), 7.09 (s, 1H), 6.60-6.57 (m, 1H), 5.75 (s, 2H), 4.90-4.78 (m, 1H), 4.68 (s, 2H), 4.20 (d, J=13.2 Hz, 2H), 4.01 (t, J=5.2 Hz, 1H), 3.63-3.39 (m, 16H), 3.06 (s, 2H), 2.45 (s, 3H), 2.38-2.17 (m, 7H), 2.00-1.78 (m, 6H), 1.40 (br s, 9H).

### Step 4: Synthesis of I-93

To a solution of I-93-5 (0.1 g, 92.19 μmol, 1 eq) in dioxane (0.5 mL) was added HCl/dioxane (4 M, 0.5 mL, 21.69 eq) and then the mixture was stirred at 25° C. for 0.5 hr. The reaction mixture was concentrated under room temperature reduced pressure to remove solvent to give product. The product was added 30 mL pure water and lyophilized to give I-93 (86.64 mg, 82.31 μmol, 89.28% yield, 97% purity, HCl) as an off-white solid. LCMS (Method G): R^{t}=0.576 min, [M+H]⁺=984.5. SFC: R^{t}=1.056 min. 1H NMR (400 MHZ, DMSO-d₆) δ=12.82 (br s, 1H), 11.63-11.36 (m, 1H), 9.81-9.61 (m, 2H), 9.13 (br s, 3H), 8.37 (s, 1H), 8.31 (d, J=1.6 Hz, 1H), 7.93 (t, J=1.2 Hz, 1H), 7.76 (s, 1H), 7.73-7.60 (m, 2H), 7.52-7.33 (m, 5H), 7.00 (d, J=1.2 Hz, 1H), 5.00-4.89 (m, 1H), 4.75 (s, 2H), 4.51-3.95 (m, 8H), 3.65-3.51 (m, 12H), 3.42 (s, 2H), 3.29-3.08 (m, 5H), 3.06 (s, 2H), 2.73-2.56 (m, 4H), 2.50-2.47 (m, 3H), 2.40-2.28 (m, 1H), 2.26-2.09 (m, 2H), 1.98-1.96 (m, 1H). ¹H NMR (400 MHZ, DMSO+D2O) δ=9.45 (d, J=1.2 Hz, 1H), 8.31 (s, 1H), 8.08 (s, 1H), 7.73 (s, 1H), 7.66-7.59 (m, 2H), 7.52 (br d, J=8.4 Hz, 1H), 7.41-7.31 (m, 5H), 6.90 (br d, J=3.4 Hz, 1H), 4.89-4.88 (m, 1H), 4.65 (s, 2H), 4.34-4.32 (m, 2H), 3.82-3.81 (m, 8H), 3.60-3.36 (m, 10H), 3.33 (s, 2H), 3.19-3.02 (m, 3H), 3.00 (s, 2H), 2.98-2.89 (m, 1H), 2.75-2.51 (m, 4H), 2.45 (s, 3H), 2.33-2.19 (m, 1H), 2.18-2.02 (m, 2H), 1.93-1.90 (m, 1H),

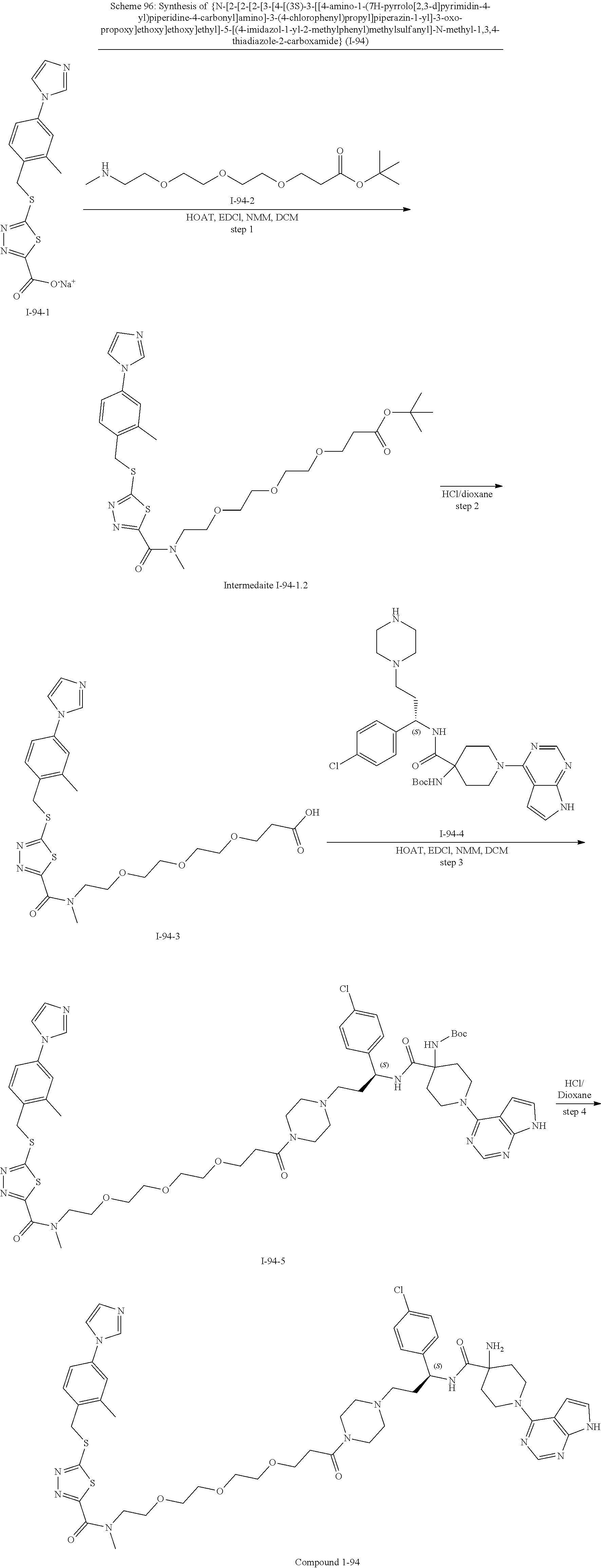

### Step 1: Synthesis of I-94-1.2

To a mixture of I-94-2 (901.88 mg, 3.10 mmol, 1.1 eq), I-94-1 (1 g, 2.81 mmol, 1 eq, sodium salt), HOAt (574.48 mg, 4.22 mmol, 1.5 eq), EDCI (1.62 g, 8.44 mmol, 3 eq) in DCM (10 mL) was added NMM (1.42 g, 14.07 mmol, 1.55 mL, 5 eq), the reaction mixture was stirred for 3 hr at 25° C., The reaction mixture was quenched with water (10 mL) and then extracted with DCM (5 mL*3), the organic phase was dried by anhydrous Na₂SO₄, filtered to give filtrates and then concentrates to give crude product. The filtrates were purified by reverse phase purification (120 g of SepaFlash® Spherical C18, 20-45 μm, 100 Å; mobile phase: A for H2O+0.1% NH₃·H2O and B for acetonitrile; gradient: B 50%-60% in 40 min, flow rate: 150 mL/min; column temperature: RT, wavelength: 220 nm/254 nm), after purification, then concentrated to remove organic solvents, the residual aqueous solution was lyophilized to give I-94-1.2 (1.65 g, 2.72 mmol, 96.66% yield, 99.855% purity) as yellow oil. LCMS (Method G): R^{t}=0.659 min, [M+H]⁺=606.1. ¹H NMR (400 MHZ, DMSO-d₆) δ=8.24 (s, 1H), 7.73 (s, 1H), 7.60-7.53 (m, 2H), 7.46-7.40 (m, 1H), 7.09 (s, 1H), 4.69 (s, 2H), 4.02 (t, J=5.4 Hz, 1H), 3.64-3.41 (m, 15H), 3.06 (s, 2H), 2.46 (s, 3H), 2.39 (t, J=6.4 Hz, 2H), 1.38 (s, 8H).

### Step 2: Synthesis of I-94-3

To a solution of I-94-1.2 (100 mg, 165.08 μmol, 1 eq) in HCl/dioxane (1 mL), then the mixture was stirred at 25° C. for 1 h. The reaction mixture was concentrated reduced pressure to remove solvent to give I-94-3 (100 mg, crude) as yellow oil. The crude product was used into next step directly without any other purification. LCMS (Method E): R^{t}=0.410 min, [M+H]⁺=550.2.

### Step 3: Synthesis of I-94-5

To a solution of I-94-4 (101.88 mg, 170.61 μmol, 1 eq), I-94-3 (100 mg, crude, HCl), HOAt (23.22 mg, 170.61 μmol, 1 eq), EDCI (98.12 mg, 511.84 μmol, 3 eq) in DMF (1 mL) was added NMM (86.29 mg, 853.06 μmol, 93.79 μL, 5 eq), the reaction mixture was stirred at 25° C. for 2 hr. The reaction mixture was filtered to give filtrates. The filtrates were purified by reverse phase purification (80 g of SepaFlash® Spherical C18, 20-45 μm, 100 Å; mobile phase: A for H₂O+0.1% NH₃·H₂O and B for acetonitrile; Gradient: B 55%-60% in 15 min, flow rate: 150 mL/min; column temperature: RT, wavelength: 220 nm/254 nm), after purification, then concentrated to remove organic solvents, the residual aqueous solution was lyophilized to give I-94-5 (100 mg, 88.59 μmol, 51.92% yield) as yellow gum. LCMS (Method G): R^{t}=0.649 min, [M+H]⁺=1128.7. SFC: R^{t}=0.751 min. ¹H NMR (400 MHZ, DMSO-d6) δ=11.65 (br s, 1H), 8.33-8.21 (m, 2H), 8.11 (s, 1H), 7.72 (t, J=1.4 Hz, 1H), 7.60-7.52 (m, 2H), 7.49-7.42 (m, 1H), 7.31 (s, 4H), 7.21-7.12 (m, 2H), 7.09 (s, 1H), 6.59-6.58 (m, 1H), 4.94-4.79 (m, 1H), 4.68 (s, 2H), 4.28-4.13 (m, 2H), 4.02 (t, J=5.4 Hz, 1H), 3.63-3.57 (m, 5H), 3.52-3.39 (m, 13H), 3.06 (s, 1H), 2.55-2.51 (m, 4H), 2.45 (s, 3H), 2.4-2.13 (m, 8H), 2.00-1.74 (m, 6H), 1.41 (br s, 9H).

### Step 4: Synthesis of I-94

To a solution of I-94-5 (100 mg, 88.59 μmol, 1 eq) in dioxane (0.5 mL) was added HCl/dioxane (0.5 mL), the reaction mixture was stirred at 25° C. for 0.5 hr. The reaction mixture was concentrated under room temperature to give crude product. The crude product was purified by reverse phase purification (80 g of SepaFlash® Spherical C18, 20-45 μm, 100 Å; mobile phase: A for H₂O+0.1% NH₃·H₂O and B for acetonitrile; gradient: B 50%-60% in 15 min, flow rate: 150 mL/min; column temperature: RT, wavelength: 220 nm/254 nm), after purification, then concentrated to remove organic solvents, the residual aqueous solution was lyophilized to I-94 (35 mg, 34.02 μmol, 38.41% yield, 100% purity) as a white solid. LCMS (Method G): R^{t}=0.617 min, [M+H]⁺=1028.5. SFC: R^{t}=1.109 min. _F2. ¹H NMR (400 MHZ, DMSO+D2O) δ=8.23 (s, 1H), 8.09 (s, 1H), 7.71 (s, 1H), 7.59-7.51 (m, 2H), 7.43-7.30 (m, 1H), 7.38-7.26 (m, 4H), 7.17-7.05 (m, 2H), 6.56 (d, J=3.6 Hz, 1H), 4.81 (t, J=6.4 Hz, 1H), 4.65 (s, 2H), 4.37 (t, J=11.6 Hz, 2H), 3.99 (t, J=4.8 Hz, 1H), 3.61 (s, 3H), 3.52-3.34 (m, 16H), 3.04 (s, 2H), 2.53-2.51 (m, 2H), 2.44 (s, 3H), 2.38-2.13 (m, 7H), 1.97-1.78 (m, 4H), 1.41 (t, J=14.4 Hz, 2H).

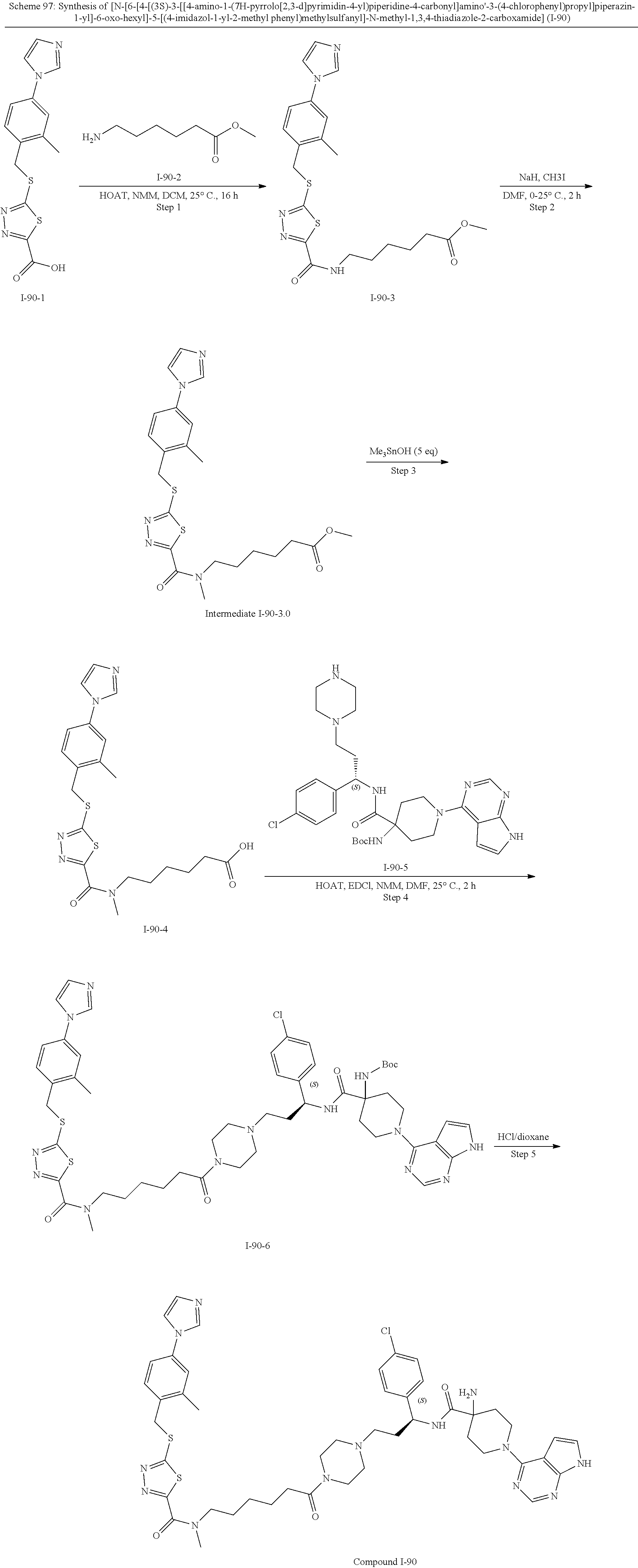

### Step 1: Synthesis of I-90-3

To a solution of I-90-1 (1 g. 2.81 mmol, 1 eq. sodium salt) in DCM (15 mL) was added I-90-2 (562.27 mg, 3.10 mmol, 1.1 eq). HOAt (574.48 mg, 4.22 mmol, 1.5 eq). EDCI (1.62 g. 8.44 mmol, 3 eq). NMM (1.42 g, 14.07 mmol, 1.55 mL, 5 eq), then the mixture was stirred at 25° C. for 1 hr. The reaction mixture was quenched with water (20 mL), extracted with DCM (10 mL*3), the organic phase was dried by anhydrous Na₂SO₄, filtered and the filtrates was concentrated to give crude. The crude product was triturated with (PE:EA=3:1, 20 mL), then filtered to give I-90-3 (1.2 g, 2.61 mmol, 92.79% yield, N/A purity) as a white solid. LCMS (Method E): R^{t}=0.825 min, [M+H]⁺=460.1. ¹H NMR (400 MHZ, DMSO-d6) δ=9.21 (s, 1H), 8.24 (s, 1H), 7.73 (s, 1H), 7.59-7.52 (m, 2H), 7.45-7.43 (m, 1H), 7.09 (s, 1H), 4.68 (s, 2H), 3.56 (s, 3H), 3.24 (d, J=6.4 Hz, 2H), 2.46 (s, 3H), 2.29 (t, J=7.4 Hz, 2H), 1.63-1.44 (m, 4H), 1.38-1.20 (m, 2H).

### Step 2: Synthesis of I-90-3.0

To a solution of I-90-3 (1 g, 2.18 mmol, 1 eq) in DMF (10 mL) was added NaH (130.54 mg, 3.26 mmol, 60% purity, 1.5 eq) at 0° C. stirred for 1 h under N₂, then MeI (308.84 mg, 2.18 mmol, 135.46 μL, 1.0 eq) was added to the mixture and then stirred at 0-25° C. for 1 h under N₂. The reaction mixture was quenched with ice saturated solution of NH₄Cl (10 mL) and then extracted with EA (10 mL*3), the organic phase was washed by brine (20 mL*2), the combined organic phase was dried by anhydrous Na₂SO₄, filtered to give filtrates and then concentrates to give crude product. The crude product was purified by reverse phase purification (105 g of SepaFlash® Spherical C18, 20-45 μm, 100 Å; mobile phase: A for H2O+0.1% NH₃·H2O and B for acetonitrile; gradient: B 40-50% in 30 min, flow rate: 100 mL/min; column temperature: RT, wavelength: 220 nm/254 nm) to give I-90-3.0 (520 mg, 1.07 mmol, 49.28% yield, 97.671% purity) as an off-white solid. LCMS (Method G): R^{t}=0.613 min, [M+H]⁺=474.1. SFC data: R^{t}=0.906 min. _F1. ¹H NMR (400 MHZ, DMSO-d₆) δ=8.24 (s, 1H), 7.73 (t, J=1.4 Hz, 1H), 7.59-7.53 (m, 2H), 7.49-7.43 (m, 1H), 7.09 (s, 1H), 4.69 (d, J=2.4 Hz, 2H), 3.87-3.74 (m, 1H), 3.57 (d, J=1.8 Hz, 3H), 3.45 (t, J=7.4 Hz, 1H), 3.38-3.34 (m, 2H), 3.05-3.00 (m, 2H), 2.46 (s, 3H), 2.30 (q, J=7.4 Hz, 2H), 1.68-1.46 (m, 4H), 1.35-1.13 (m, 2H). ¹H NMR (400 MHZ, DMSO+D2O) δ=8.20 (s, 1H), 7.67 (s, 1H), 7.55-7.49 (m, 2H), 7.40 (d, J=8.0 Hz, 1H), 7.08 (s, 1H), 4.63 (s, 2H), 3.77-3.70 (m, 1H), 3.53 (d, J=3.2 Hz, 3H), 3.42 (t, J=7.4 Hz, 1H), 3.32-2.94 (m, 3H), 2.43 (s, 3H), 2.32-2.16 (m, 2H), 1.64-1.42 (m, 4H), 1.31-1.10 (m, 2H). ¹H NMR (400 MHZ, DMSO-d₆) δ=8.16 (s, 1H), 7.65 (s, 1H), 7.58-7.49 (m, 2H), 7.42-7.41 (m 1H), 7.09 (s, 1H), 4.69 (s, 2H), 3.90-3.72 (m, 1H), 3.59 (s, 3H), 3.48 (br s, 1H), 3.36 (br s, 1H), 3.04 (br s, 1H), 2.48 (s, 3H), 2.30 (t, J=6.4 Hz, 2H), 1.61-1.60 (m, 4H), 1.30 (d, J=1.6 Hz, 2H).

### Step 3: Synthesis of I-90-4

To a solution of I-90-3.0 (200 mg, 422.29 μmol, 1 eq) in DCE (2 mL) was added Me₃SnOH (381.79 mg, 2.11 mmol, 5 eq) and then the reaction mixture was stirred at 80° C. for 16 hr, The reaction mixture was worked up with another batch. The reaction mixture was diluted with water (2 mL) then extracted with DCM (2 mL*3), the organic phase was dried by anhydrous Na₂SO₄, filtered to give filtrates and then concentrates to give crude product. The crude product was purified by reverse phase purification (40 g of SepaFlash® Spherical C18, 20-45 μm, 100; mobile phase: A for H2O+0.1% NH₃·H2O and B for acetonitrile; gradient: B 10 30% in 30 min, flow rate: 100 mL/min; column temperature: RT, wavelength: 220 nm/254 nm) to give I-90-4 (60 mg, 114.89 μmol, 27.21% yield, 88% purity) as a white solid. LCMS (Method E): R^{t}=0.418 min, [M+H]⁺=460.3.

### Step 4: Synthesis of I-90-6

To a solution of I-90-4 (60 mg, 130.55 μmol, 1 eq), I-90-5 (77.96 mg, 130.55 μmol, 1 eq), HOAt (17.77 mg, 130.55 μmol, 1 eq), EDCI (75.08 mg, 391.66 μmol, 3 eq) in DMF (1 mL) was added NMM (66.03 mg, 652.76 μmol, 71.77 μL, 5 eq), the reaction mixture was stirred at 25° C. for 12 hr, The reaction mixture was filtered to give filtrates. The filtrates were purified by reverse phase purification (80 g of SepaFlash® Spherical C18, 20-45 μm, 100 Å; mobile phase: A for H2O+0.1% NH₃·H2O and B for acetonitrile; gradient: B 50%-60% in 30 min, flow rate: 150 mL/min; column temperature: RT, wavelength: 220 nm/254 nm) and re-purified by (80 g of SepaFlash® Spherical C18, 20-45 μm, 100 Å; mobile phase: A for H2O+0.1% FA and B for acetonitrile; gradient: B 40%-50% in 30 min, flow rate: 150 mL/min; column temperature: RT, wavelength: 220 nm/254 nm) to give I-90-6 (40 mg, 38.51 μmol, 29.50% yield) as a white solid. LCMS (Method E): R^{t}=0.415 min, [M+H]⁺=1038.5. SFC data: R^{t}=0.792 min. ¹H NMR (400 MHz, DMSO-d₆) δ=11.67 (br s, 1H), 8.35-8.21 (m, 2H), 8.12 (s, 1H), 7.73 (d, J=1.4 Hz, 1H), 7.57 (s, 1H), 7.49-7.43 (m, 1H), 7.33-7.28 (m, 4H), 7.22-7.06 (m, 3H), 6.59 (br s, 1H), 4.94-4.81 (m, 1H), 4.68 (s, 2H), 4.30-4.11 (m, 2H), 3.86-3.75 (m, 1H), 3.68-3.53 (m, 2H), 3.46 (br s, 5H), 3.02 (s, 2H), 2.45 (d, J=2.8 Hz, 2H), 2.42-2.16 (m, 10H), 2.05-1.74 (m, 7H), 1.71-1.49 (m, 4H), 1.41 (br s, 8H), 1.27-1.25 (m, 3H).

### Step 5: Synthesis of 1-90

To a solution I-90-6 of (40 mg, 38.51 μmol, 1 eq) in dioxane (0.5 mL) was added HCl/dioxane (4N, 0.5 mL), the reaction mixture was stirred at 25° C. for 0.5 hr. The reaction mixture was concentrated under room temperature to give product. The product was added 40 mL pure water and lyophilized to give I-90 (20 mg, 20.99 μmol, 54.50% yield, 98.489% purity) as a yellow solid. LCMS (Method E): R^{t}=0.398 min, [M+H]⁺=938.5. SFC: R^{t}=1.185 min. ¹H NMR (400 MHz, DMSO-d₆) δ=12.92-12.48 (m, 1H), 11.74-11.29 (m, 1H), 9.81-9.54 (m, 2H), 9.10 (br s, 3H), 8.40-8.24 (m, 2H), 7.91 (s, 1H), 7.75 (s, 1H), 7.72-7.61 (m, 2H), 7.43 (q, J=8.4 Hz, 5H), 6.95 (br s, 1H), 4.95 (br s, 1H), 4.75 (s, 2H), 4.46-3.96 (m, 7H), 3.83-3.78 (m, 1H), 3.52-3.41 (m, 4H), 3.36 (s, 2H), 3.28-3.07 (m, 4H), 3.02 (s, 2H), 3.01-2.80 (m, 2H), 2.66-2.51 (m, 4H), 2.46-2.03 (m, 6H), 2.02-1.91 (m, 1H), 1.67-1.44 (m, 4H), 1.32-1.22 (m, 2H)

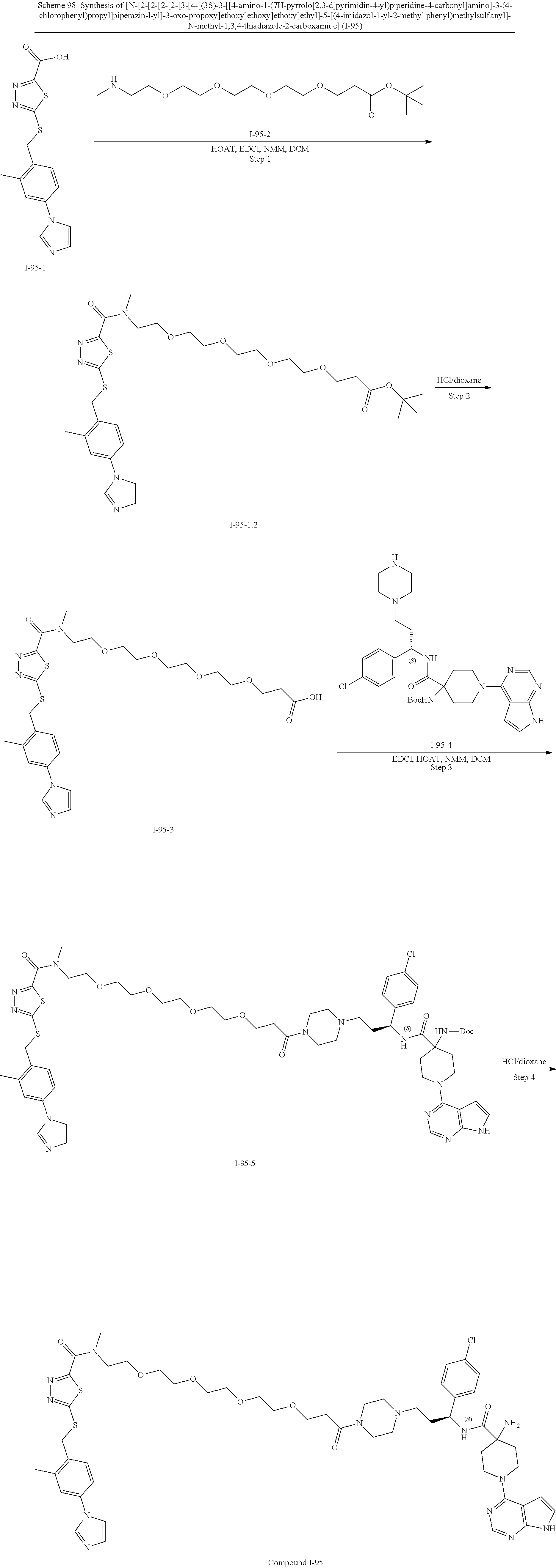

### Step 1: Synthesis of I-95-1.2

To a solution of I-95-1 (900 mg, 2.71 mmol, 1 eq sodium salt), I-95-2 (908.22 mg, 2.71 mmol, 1 eq), HOAt (552.80 mg, 4.06 mmol, 1.5 eq), EDCI (1.56 g, 8.12 mmol, 3 eq) in DCM (9 mL) was added NMM (1.37 g, 13.54 mmol, 1.49 mL, 5 eq), the reaction mixture was stirred for 3 hrs at 25° C. The reaction mixture was quenched with water (10 mL) and then extracted with DCM (5 mL*3). The organic phase was dried over anhydrous Na₂SO₄, filtered and the filtrate was concentrated to give crude product. The crude product was purified by reverse phase purification (120 g of SepaFlash® Spherical C18, 20-45 μm, 100 Å; mobile phase: A for H₂O+0.1% NH₃·H₂O and B for acetonitrile; gradient: B 60%-80% in 40 min, flow rate: 150 mL/min; column temperature: RT, wavelength: 220 nm/254 nm) to give I-95-1.2 (1.52 g, 2.33 mmol, 86.02% yield, 99.574% purity) yellow oil. LCMS (Method G): R^{t}=0.654 min, [M+H]⁺=650.1. ¹H NMR (400 MHZ, DMSO-d₆) δ=8.24 (s, 1H), 7.73 (t, J=1.4 Hz, 1H), 7.59-7.53 (m, 2H), 7.46-7.40 (m, 1H), 7.09 (t, J=1.2 Hz, 1H), 4.69 (s, 2H), 4.02 (t, J=5.4 Hz, 1H), 3.65-3.41 (m, 19H), 3.07 (s, 2H), 2.46 (s, 3H), 2.40 (t, J=6.4 Hz, 2H), 1.38 (s, 8H).

### Step 2: Synthesis of I-95-5

A solution of I-95-1.2 (100 mg, 153.89 μmol, 1 eq) in HCl/dioxane (1 mL), then the mixture was stirred at 25° C. for 1 h. The reaction mixture was concentrated to give I-95-3 (100 mg, crude, HCl) as yellow oil. The crude product was used into next step directly without any other purification. LCMS (Method E): R^{t}=0.417 min, [M+H]⁺=594.3.

### Step 3: Synthesis of I-95-5

To a solution of I-95-4 (94.76 mg, 158.69 μmol, 1 eq), Synthesis of I-95-3 (100 mg, 158.69 μmol, 1 eq, HCl), HOAt (21.60 mg, 158.69 μmol, 1 eq), EDCI (91.26 mg, 476.06 μmol, 3 eq) in DMF (1 mL) was added NMM (80.26 mg, 793.43 μmol, 87.23 μL, 5 eq), the reaction mixture was stirred at 25° C. for 2 hr. The reaction mixture was filtered and the filtrates were purified by reverse phase purification (80 g of SepaFlash® Spherical C18, 20-45 μm, 100 Å; mobile phase: A for H2O+0.1% NH₃·H2O and B for acetonitrile; gradient: B 50%-60% in 20 min, flow rate: 150 mL/min; column temperature: RT, wavelength: 220 nm/254 nm) to give I-95-5 (110 mg, 93.79 μmol, 59.10% yield) as a white solid. LCMS (Method G): R^{t}=0.651 min, [M+H]⁺=1172.7. SFC R^{t}=0.754 min. ¹H NMR (400 MHZ, DMSO-d₆) δ=11.66 (br s, 1H), 8.33-8.21 (m, 2H), 8.12 (s, 1H), 7.73 (t, J=1.4 Hz, 1H), 7.61-7.52 (m, 2H), 7.49-7.42 (m, 1H), 7.31 (s, 4H), 7.22-7.13 (m, 2H), 7.09 (s, 1H), 6.59-6.58 (m 1H), 4.94-4.80 (m, 1H), 4.68 (s, 2H), 4.32-4.12 (m, 2H), 4.02 (t, J=5.4 Hz, 1H), 3.66-3.57 (m, 6H), 3.56-3.37 (m, 18H), 3.06 (s, 1H), 2.56-2.51 (m, 4H), 2.45 (s, 3H), 2.40-2.10 (m, 7H), 2.02-1.77 (m, 6H), 1.41 (br s, 7H),

### Step 4: Synthesis of I-95

To a solution of I-95-5 (110 mg, 93.79 μmol, 1 eq) in dioxane (0.5 mL) was added HCl/dioxane (4M, 0.5 mL), the reaction mixture was stirred at 25° C. for 0.5 hr. The reaction mixture was concentrated under room temperature to give crude product. The crude product was purified by reverse phase purification (80 g of SepaFlash® Spherical C18, 20-45 μm, 100 Å; mobile phase: A for H2O+0.1% NH₃·H2O and B for acetonitrile; gradient: B 50%-60% in 15 min, flow rate: 150 mL/min; column temperature: RT, wavelength: 220 nm/254 nm) to give I-95 (70 mg, 65.25 μmol, 69.58% yield, 100% purity) as a white solid. LCMS (Method G): R^{t}=0.623 min, [M+H]⁺=1072.6. SFC R^{t}=1.107 min. ¹H NMR (400 MHZ, DMSO-d6) δ=11.66 (br s, 1H), 8.78 (d, J=8.0 Hz, 1H), 8.25 (s, 1H), 8.11 (s, 1H), 7.73 (s, 1H), 7.60-7.52 (m, 2H), 7.46-7.40 (m, 1H), 7.38-7.29 (m, 4H), 7.17-7.08 (m, 2H), 6.57 (d, J=1.4 Hz, 1H), 4.93-4.77 (m, 1H), 4.68 (s, 2H), 4.50-4.30 (m, 2H), 4.02 (t, J=5.2 Hz, 1H), 3.64-3.56 (m, 5H), 3.55-3.44 (m, 15H), 3.43 (br s, 1H), 3.41 (br s, 2H), 3.06 (s, 2H), 2.57-2.50 (m, 4H), 2.39-2.13 (m, 8H), 1.99-1.79 (m, 4H), 1.42 (t, J=14.0 Hz, 2H).

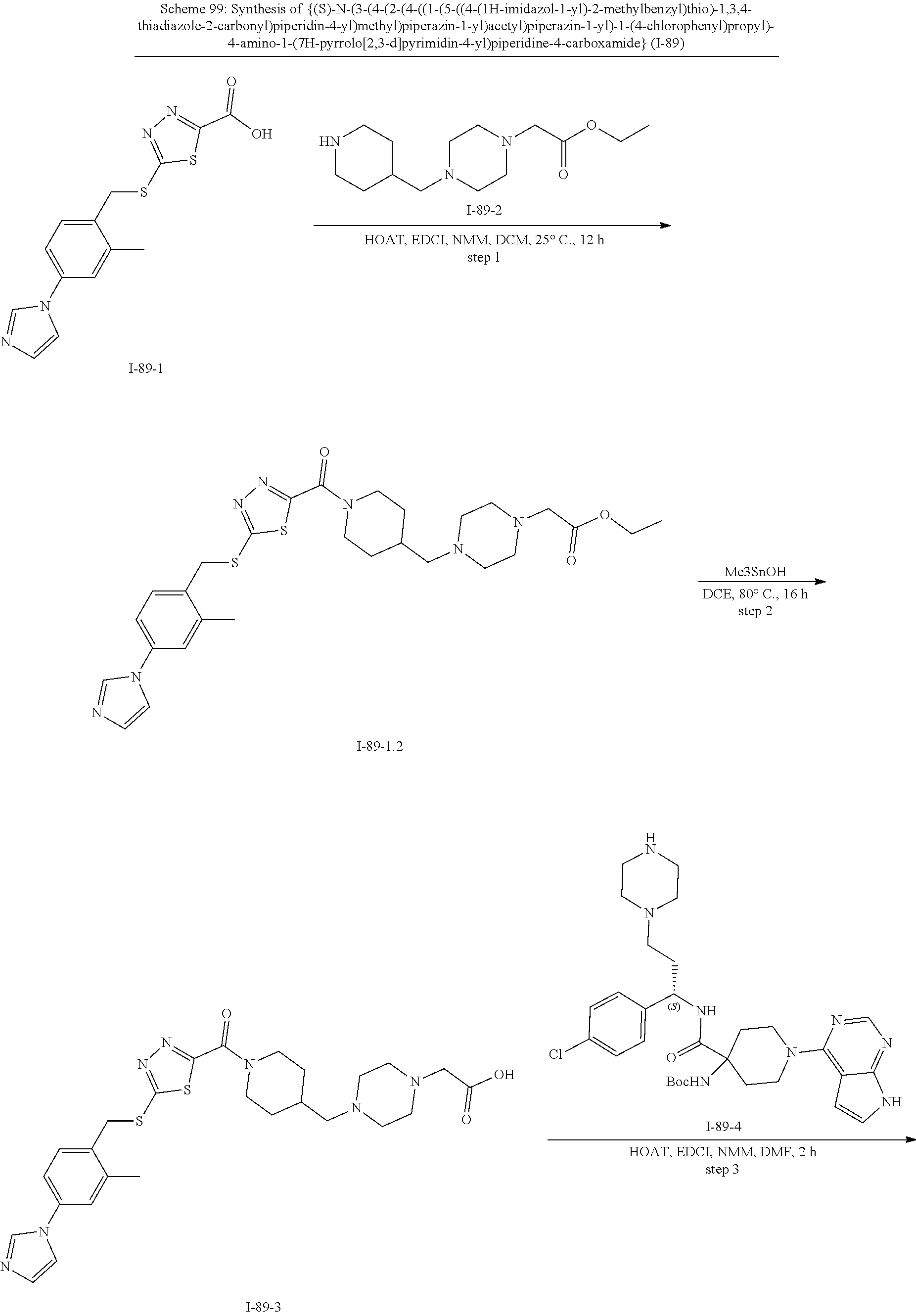

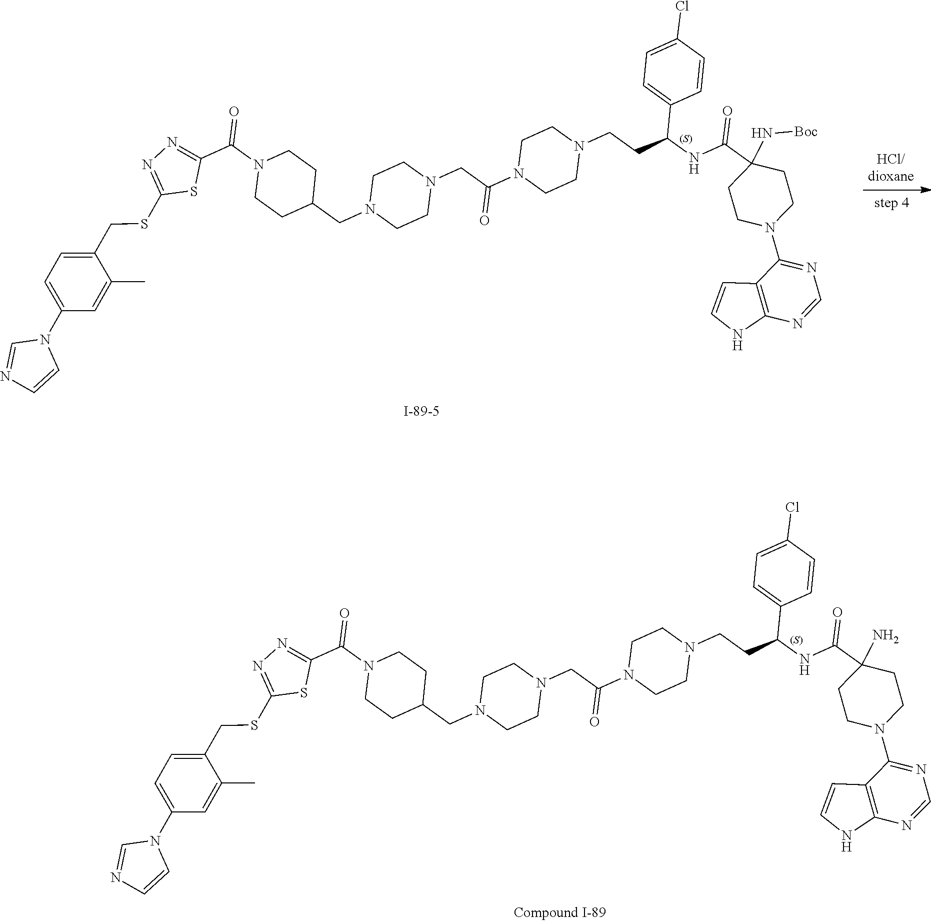

### Step 1: Synthesis of I-89-1.2

To a mixture of I-89-1 (1 g, 2.81 mmol, 1 eq, sodium salt), I-89-2 (946.64 mg, 3.10 mmol, 1.1 eq, HCl), HOAt (574.48 mg, 4.22 mmol, 1.5 eq), EDCI (1.62 g, 8.44 mmol, 3 eq) in DCM (1 mL) was added NMM (1.42 g, 14.07 mmol, 1.55 mL, 5 eq), then the mixture was stirred at 25° C. for 12 hrs. The reaction mixture was worked up with another batch, the combined reaction mixture was quenched with water (15 mL) and then extracted with DCM (10 mL*3). The organic phase was dried by anhydrous Na₂SO₄, filtered and concentrated to give crude product. The crude product was purified by reverse phase purification (80 g of SepaFlash® Spherical C18, 20-45 μm, 100 Å; mobile phase: A for H2O+0.1% NH₃·H2O and B for acetonitrile; gradient: B 40-50% in 25 min, flow rate: 100 mL/min; column temperature: RT, wavelength: 220 nm/254 nm), then concentrated to remove organic solvents, the residual aqueous solution was lyophilized to give I-89-1.2 (930 mg, 1.56 mmol, 55.34% yield, 97.749% purity) as a yellow solid. LCMS (Method C): R^{t}=0.816 min, [M+H]⁺=584.1. ¹H NMR (400 MHZ, DMSO-d₆) δ=8.21 (s, 1H), 7.68 (s, 1H), 7.57-7.49 (m, 2H), 7.41-7.39 (m, 1H), 7.08 (s, 1H), 4.68-4.54 (m, 3H), 4.34 (d, J=12.4 Hz, 1H), 4.05 (q, J=7.2 Hz, 2H), 3.24-3.06 (m, 3H), 2.93-2.77 (m, 1H), 2.49-2.41 (m, 7H), 2.33 (br s, 4H), 2.11 (d, J=6.8 Hz, 2H), 1.92-1.67 (m, 3H), 1.23-0.93 (m, 5H).

### Step 2: Synthesis of I-89-3

To a solution of I-89-1.2 (200 mg, 342.60 μmol, 1 eq) in DCE (2 mL) was added hydroxy (trimethyl) stannane (309.75 mg, 1.71 mmol, 5 eq), the reaction mixture was stirred at 80° C. for 16 hrs. The reaction mixture was filtered and the filter cake was washed with DCM (0.5 mL*3), the filtrates was concentrated to give crude product. The crude product was purified by flash silica gel chromatography (ISCO®; 10 g SepaFlash® Silica Flash Column, Eluent of 0˜100% MeOH/EA @ 60 mL/min), then concentrated to give product. I-89-3 (150 mg, 186.25 μmol, 54.36% yield, 69% purity) was obtained as yellow gum. LCMS (Method E): R^{t}=0.362 min, [M+H]⁺=556.3. ¹H NMR (400 MHZ, DMSO-d₆) δ=8.25 (s, 1H), 7.96 (s, 1H), 7.74 (s, 1H), 7.56 (d, J=8.4 Hz, 2H), 7.46-7.40 (m, 1H), 7.10 (s, 1H), 4.77-4.63 (m, 3H), 4.39 (d, J=13.4 Hz, 1H), 3.21 (br s, 1H), 2.89 (s, 3H), 2.73 (s, 3H), 2.47 (s, 3H), 2.42-2.30 (m, 4H), 2.13 (d, J=6.8 Hz, 2H), 1.88-1.74 (m, 3H), 1.17-1.02 (m, 2H),

### Step 3: Synthesis of I-89-5

To a solution of I-89-4 (111.22 mg, 186.25 μmol, 1 eq), I-89-3 (150 mg, 186.25 μmol, 1 eq), HOAt (25.35 mg, 186.25 μmol, 1 eq), EDCI (107.11 mg, 558.74 μmol, 3 eq) in DMF (1 mL) was added NMM (94.19 mg, 931.23 μmol, 102.39 μL, 5 eq), the reaction mixture was stirred at 25° C. for 2 hrs. The reaction mixture was filtered. The filtrates were purified by reverse phase purification (80 g of SepaFlash® Spherical C18, 20-45 μm, 100 Å; mobile phase: A for H2O+0.1% NH₃·H2O and B for acetonitrile; gradient: B 40%-50% in 30 min, flow rate: 150 mL/min; column temperature: RT, wavelength: 220 nm/254 nm), then concentrated to remove organic solvents, the residual aqueous solution was lyophilized to give I-89-5 (80 mg, 70.49 μmol, 37.85% yield) as a white solid. LCMS (Method E): R^{t}=0.396 min, [M+H]⁺=1134.6. SFC: R^{t}=1.186 min. ¹H NMR (400 MHz, DMSO-d₆) δ=11.66 (br s, 1H), 8.29-8.19 (m, 2H), 8.12 (s, 1H), 7.73 (s, 1H), 7.61-7.51 (m, 2H), 7.46-7.35 (m, 1H), 7.32 (s, 4H), 7.17-7.15 (m, 1H), 7.09 (s, 1H), 6.59 (d, J=1.4 Hz, 1H), 4.94-4.64 (m, 4H), 4.45-4.12 (m, 3H), 3.68-3.52 (m, 4H), 3.47 (br s, 2H), 3.26-3.01 (m, 5H), 2.86 (t, J=11.8 Hz, 1H), 2.46 (s, 3H), 2.43-2.20 (m, 12H), 2.11 (d, J=6.4 Hz, 2H), 2.02-1.71 (m, 9H), 1.42 (br s, 9H), 1.18-0.98 (m, 2H).

### Step 4: Synthesis of 1-89

To a solution of I-89-5 (80 mg, 70.49 μmol, 1 eq) in dioxane (1 mL) was added HCl/dioxane (1 mL), the reaction mixture was stirred at 25° C. for 0.5 hr. The reaction mixture was concentrated to give product. The product was added 40 mL pure water and lyophilized to give 1-89 (60 mg, 55.17 μmol, 78.26% yield, 98.488% purity, HCl) as an off-white solid. LCMS (Method E): R^{t}=0.372 min, [M+H]⁺=1034.5. SFC: R^{t}=2.062 min. F2. ¹H NMR (400 MHZ, DMSO-d₆) δ=12.89 (br s, 1H), 11.90-11.73 (m, 1H), 9.76 (d, J=1.2 Hz, 2H), 9.22 (d, J=1.6 Hz, 3H), 8.41-8.26 (m, 2H), 7.92 (s, 1H), 7.79-7.62 (m, 3H), 7.51-7.39 (m, 5H), 7.02 (br s, 1H), 4.99-4.90 (m, 1H), 4.74 (s, 3H), 4.44-4.17 (m, 8H), 4.02-3.94 (m, 2H), 3.85-3.42 (m, 14H), 3.36-3.06 (m, 8H), 2.93 (t, J=11.6 Hz, 1H), 2.71-2.55 (m, 2H), 2.44-2.32 (m, 1H), 2.21 (br s, 3H), 2.02 (d, J=9.6 Hz, 3H), 1.34-1.21 (m, 2H). ¹H NMR (400 MHZ, DMSO+D2O) δ=9.51 (br s, 1H), 8.40-8.33 (m, 1H), 8.12 (d, J=1.4 Hz, 1H), 7.77 (d, J=1.4 Hz, 1H), 7.65-7.60 (m, 2H), 7.59-7.52 (m, 1H), 7.43 (d, J=3.6 Hz, 1H), 7.38 (s, 4H), 6.96 (d, J=3.6 Hz, 1H), 4.98-4.63 (m, 4H), 4.47-4.10 (m, 6H), 4.00 (br s, 5H), 3.62-3.43 (m, 9H), 3.31-3.04 (m, 8H), 2.96-2.88 (m, 1H), 2.65-2.52 (m, 2H), 2.47 (s, 3H), 2.36-2.26 (m, 1H), 2.22-2.08 (m, 3H), 1.99-1.85 (m, 3H), 1.30-1.18 (m, 2H).

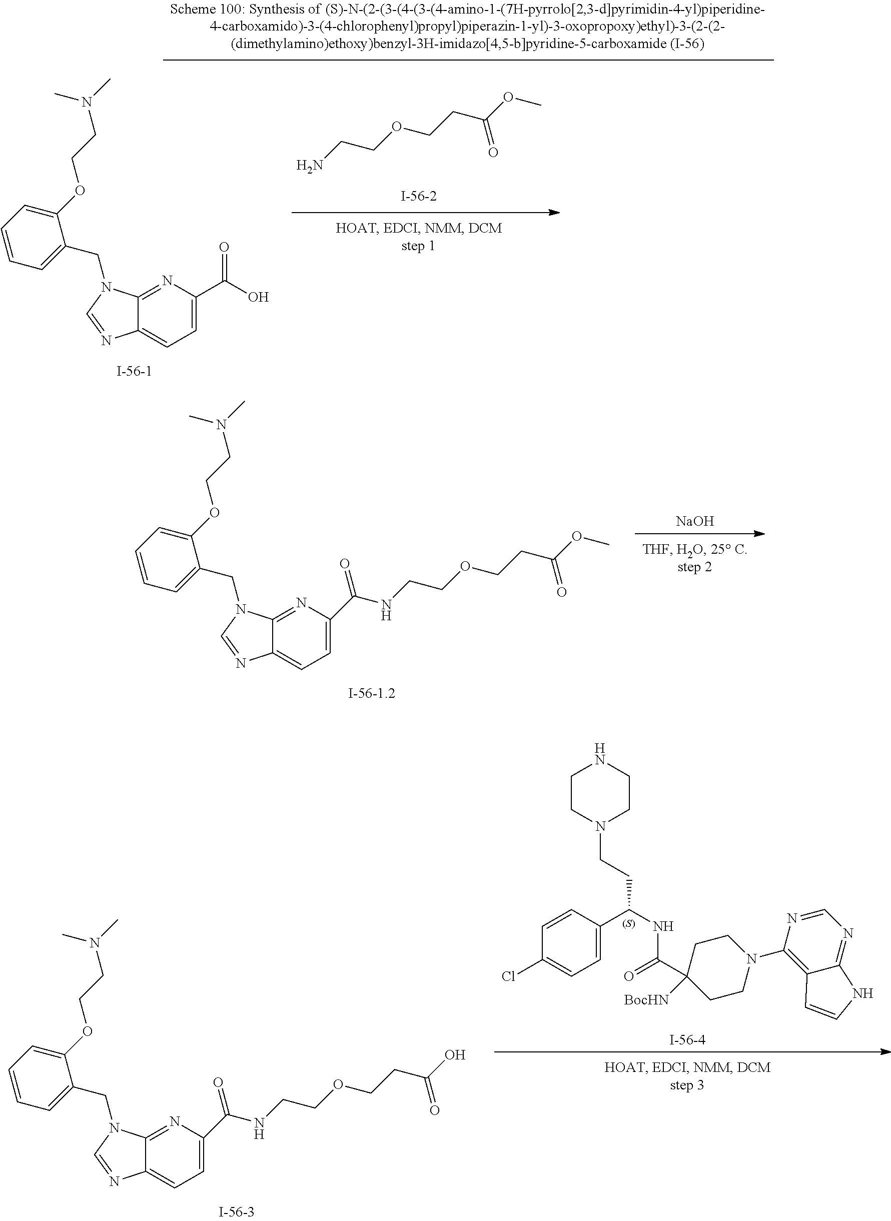

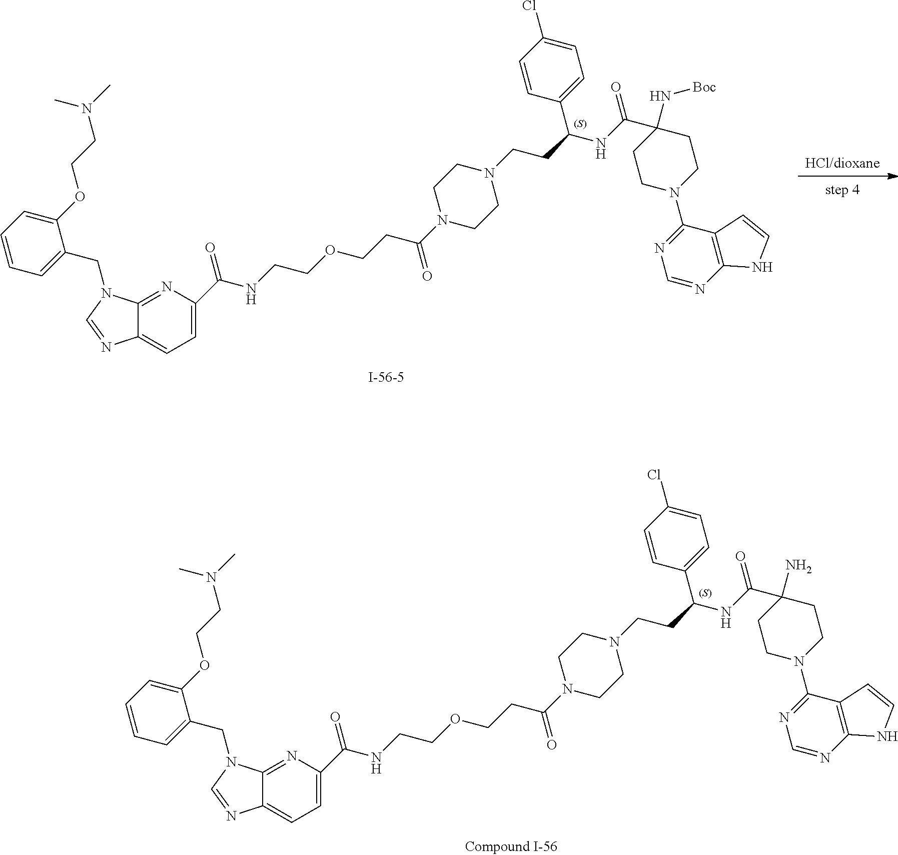

### Step 1: Synthesis of I-56-1.2

To a solution of I-56-1 (900 mg, 2.64 mmol, 1 eq) in DCM (18 mL) was added HOAt (179.95 mg. 1.32 mmol, 0.5 eq). I-56-2 (582.66 mg, 3.17 mmol, 1.2 eq. HCl). NMM (1.34 g. 13.22 mmol, 1.45 mL, 5 eq) and EDCI (1.01 g. 5.29 mmol, 2 eq). The mixture was stirred at 25° C. for 12 hr. The mixture was concentrated under vacuum to give a residue. The residue was purified by Pre-HPLC (column: Phenomenex luna C18 150*40 mm*15 um; mobile phase: [water (FA)-ACN]; gradient: 5%-35% B over 15 min) to give I-56-1.2 (800 mg, 1.60 mmol, 60.59% yield, 94.031% purity) as yellow oil. LCMS (Method G): R^{t}=0.531 min, [M+H]⁺=470.1. ¹H NMR (400 MHZ, DMSO-d₆) δ=8.76-8.68 (m, 2H), 8.18 (d, J=8.3 Hz, 1H), 8.14 (s, 1H), 7.96 (d, J=8.3 Hz, 1H), 7.45 (d, J=6.2 Hz, 1H), 7.33-7.25 (m, 1H), 7.04 (d, J=8.2 Hz, 1H), 6.91 (t, J=7.4 Hz, 1H), 5.53 (s, 2H), 4.10 (t, J=5.6 Hz, 2H), 3.70 (t, J=6.2 Hz, 2H), 3.59-3.51 (m, 7H), 2.69 (br t, J=5.5 Hz, 2H), 2.58 (t, J=6.2 Hz, 2H), 2.27 (s, 6H).

### Step 2: Synthesis of I-56-3

To a solution of I-56-1.2 (100 mg, 212.98 μmol, 1 eq) in THF (1 mL) and MeOH (1 mL) was added NaOH (1 M, 638.93 μL, 3 eq). The mixture was stirred at 25° C. for 1 h. The mixture was concentrated under vacuum to remove THE/MeOH and adjusted to pH=7 by 1 N aq. HCl (0.61 mL). The aqueous was purified by reversed-phase HPLC (0.1% FA condition) to give I-56-3 (80 mg, 175.63 μmol, 82.46% yield) as a white solid. LCMS (Method E): R^{t}=0.365 min, [M+H]⁺=456.2.

### Step 3: Synthesis of I-56-5

To a solution of I-56-3 (80 mg, 175.63 μmol, 1 eq) in DMF (2 mL) was added HOAt (11.95 mg, 87.81 μmol, 12.28 μL, 0.5 eq), I-56-4 (104.88 mg, 175.63 μmol, 1 eq), NMM (88.82 mg, 878.14 μmol, 96.55 μL, 5 eq) and EDCI (67.34 mg, 351.26 μmol, 2 eq). The mixture was stirred at 25° C. for 1 hr. To the reaction mixture was added 1 ml of MeOH and filtered. The filter was purified by Prep-HPLC (column: Waters Xbridge 150*25 mm*5 um; mobile phase: [water (NH₄HCO₃)-ACN]; gradient: 32%-62% B over 9 min) to give I-56-5 (70 mg, 62.59 μmol, 35.64% yield, 92.513% purity) as a white solid. LCMS (Method G): R^{t}=0.661 min, [M+H]⁺=1034.7.

### Step 4: Synthesis of I-56

To a solution of I-56-5 (70 mg, 67.66 μmol, 1 eq) in dioxane (0.5 mL) was added HCl/dioxane (4 M, 0.5 mL, 29.56 eq). The mixture was stirred at 25° C. for 1 h. The mixture was concentrated to give yellow oil. The yellow oil was diluted with MeOH (0.5 ml) and H₂O (20 ml) and lyophilized to give I-56 (62.06 mg, 62.74 μmol, 92.73% yield, 98.155% purity, HCl) as a yellow solid, LCMS (Method G): R^{t}=0.602 min, [M+H]⁺=934.6. SFC: R^{t}=0.786 min. ¹H NMR (400 MHZ, DMSO-d₆) δ=12.86 (br s, 1H), 11.46 (br s, 1H), 11.01 (br s, 1H), 9.66 (br d, J=7.2 Hz, 1H), 9.12 (br s, 2H), 8.90 (s, 1H), 8.80 (m, 1H), 8.38 (s, 1H), 8.24 (d, J=8.4 Hz, 1H), 8.01 (d, J=8.4 Hz, 1H), 7.49-7.39 (m, 6H), 7.35-7.29 (m, 1H), 7.08 (d, J=8.0 Hz, 1H), 7.00 (m, 1H), 6.95 (m, 1H), 5.76 (s, 2H), 4.94 (br d, J=1.6 Hz, 1H), 4.42-4.39 (m, 2H), 4.36-4.27 (m, 2H), 4.22-4.13 (m, 3H), 4.06-4.00 (m, 2H), 3.72-3.68 (m, 2H), 3.60-3.37 (m, 11H), 3.22 (br s, 1H), 3.07 (m, 2H), 2.84 (d, J=4.4 Hz, 6H), 2.66-2.59 (m, 3H), 2.38-2.28 (m, 1H), 2.23-2.09 (m, 2H), 1.99 (br d, J=13.2 Hz, 1H).

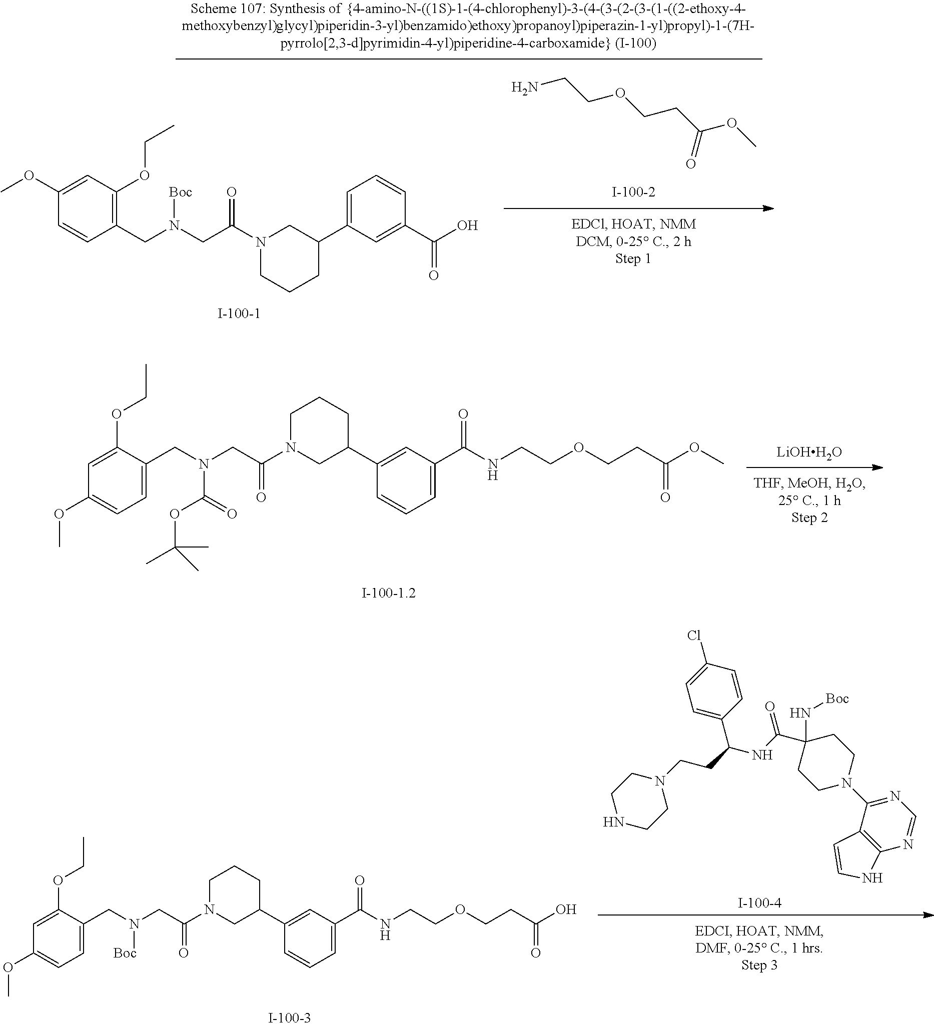

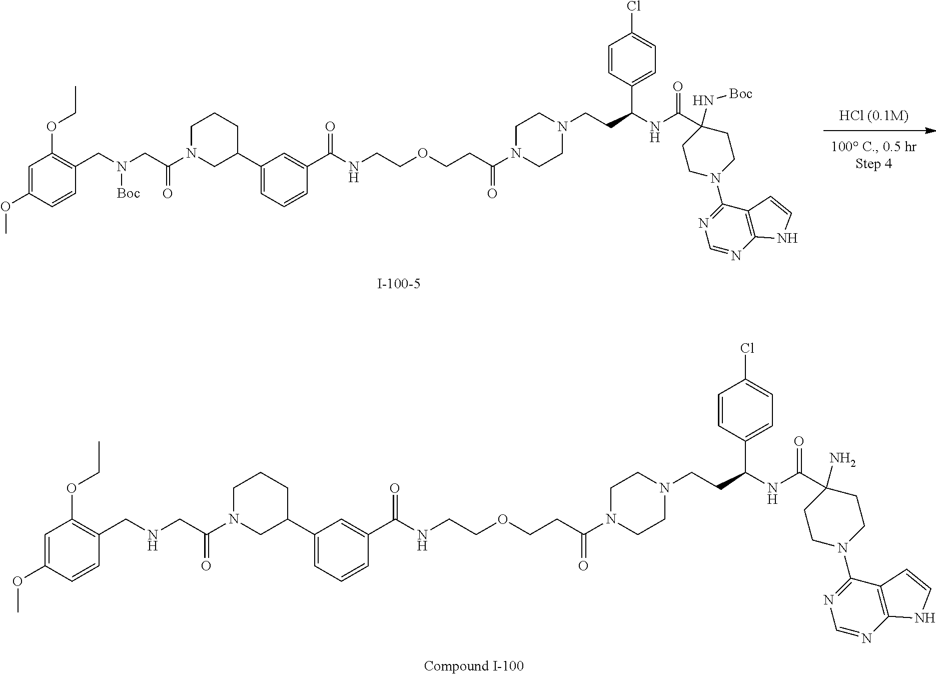 [SMILES: CCOc1cc(OC)ccc1CN(CC(=O)N1CCCC(c2cccc(C(=O)NCCOCCC(=O)N3CCN(CC[C@H](NC(=O)C4(NC(=O)OC(C)(C)C)CCN(c5ncnc6[nH]ccc56)CC4)c4ccc(Cl)cc4)CC3)c2)C1)C(=O)OC(C)(C)C ; "HCl (0.1M)" ; CCOc1cc(OC)ccc1CNCC(=O)N1CCCC(c2cccc(C(=O)NCCOCCC(=O)N3CCN(CC[C@H](NC(=O)C4(N)CCN(c5ncnc6[nH]ccc56)CC4)c4ccc(Cl)cc4)CC3)c2)C1]

### Step 1: Synthesis of I-100-1.2

To a solution of I-100-1 (1.0 g, 1.90 mmol, 1 eg) and I-100-2 (697.40 mg, 3.80 mmol, 2.0 eq, HCl) in DCM (1 mL) was added EDCI (1.09 g, 5.70 mmol, 3 eq), HOAt (387.69 mg, 2.85 mmol, 1.5 eq). The resulting mixture was cooled to 0° C. via ice-bath. Then NMM (1.92 g, 18.99 mmol, 2.09 mL, 10 eq) was introduced into the system drop-wisely. The mixture was stirred at 25° C. for 2 hrs. The reaction mixture was quenched by water (50 mL) at under ice-bath, and extracted with DCM 60 mL (20 mL*3). The combined organic layers were washed with brine (30 mL*2) and dried over anhydrous Na₂SO₄, then filtered and concentrated under reduced pressure to give the crude. The crude was purified by Flash Silica-gel chromatography (ISCO®; 20 g SepaFlash® Silica Flash Column, Eluent of 99-100% EA/PE) to give I-100-1.2 (0.9 g, 1.37 mmol, 72.27% yield, 100% purity) as colorless oil. LCMS (Method E): R^{t}=0.573 min, [M+Na]⁺=678.4. ¹H NMR (400 MHZ, CHLOROFORM-d) δ=7.83-7.57 (m, 2H), 7.42-7.28 (m, 2H), 7.23-7.06 (m, 1H), 6.92-6.78 (m, 1H), 6.52-6.33 (m, 2H), 4.79-4.64 (m, 1H), 4.49-4.41 (m, 1H), 4.07-3.92 (m, 3H), 3.83-3.72 (m, 6H), 3.66 (s, 7H), 3.10-2.94 (m, 1H), 2.84-2.47 (m, 4H), 2.08 (br s, 1H), 1.88-1.70 (m, 4H), 1.64-1.54 (m, 1H), 1.51-1.33 (m, 12H).

### Step 2: Synthesis of I-100-3

To a solution of I-100-1.2 (0.15 g, 228.74 μmol, 1 eq) in THF (0.5 mL), MeOH (0.5 mL) and H₂O (0.5 mL) was added LiOH·H₂O (47.99 mg, 1.14 mmol, 5 eq). The mixture was stirred at 25° C. for 1 hr. The reaction mixture was quenched with HCl (10 ml, 1M, Aqueous), then extracted with EA (5 mL*3). The combined organic layer was dried by anhydrous Na₂SO₄ and concentrated under reduced pressure to afford I-100-3 (0.11 g, 169.96 μmol, 74.30% yield, 99.156% purity) as colorless oil. LCMS (Method E): R^{t}=0.538 min, [M+H]⁺=642.4.

### Step 3: Synthesis of I-100-5

To a solution of I-100-3 (0.11 g, 171.41 μmol, 1.1 eq) in DMF (1.5 mL), was added EDCI (89.62 mg, 467.47 μmol, 3 eq), HOAt (31.81 mg, 233.74 μmol, 1.5 eq) and I-100-2 (93.05 mg, 155.82 μmol, 1.0 eq). Then NMM (157.61 mg, 1.56 mmol, 171.32 μL, 10 eq) was introduced into the system drop-wisely, the resulting mixture was allowed to stirred at 25° C. for 1 hr. The reaction mixture was purified by revers-phase prep-HPLC (basic condition, Combine flash (26.8*125 mm, 80 g of XB—C18, 20-40 μm, 120 Å) Mobile phase: A for H₂O (0.1% NH₃·H₂O v/v) and B for acetonitrile; Gradient: B 60%-70% in 20 min; Flow rate: 60 ml/min; Column temperature: R.T. Wavelength: 220 nm/254 nm), the desired peak was collected and concentrated to remove acetonitrile and extracted with EA (30 mL*3) to give I-100-5 (0.11 g, 72.98 μmol, 46.83% yield, 81% purity) as a white solid. LCMS (Method E): R^{t}=0.734 min, [M+H]⁺=1220.8.

### Step 4: Synthesis of I-100

The mixture of I-100-5 (0.11 g, 90.10 μmol, 1 eq) and HCl (0.1 M, 1.98 mL, 2.2 eq) was heated to 100° C., and stirred for 0.5 hrs. The reaction mixture was lyophilized directly to afford the crude. The crude was purified by prep-HPLC (Basic condition, column: Waters Xbridge 150*25 mm*5 um; mobile phase: [water (ammonia hydroxide v/v)-ACN]; gradient: 25%-55% B) then lyophilized to afford I-100 (0.03 g, 27.63 μmol, 30.67% yield, 94% purity) as a white solid. LCMS (Method E): R^{t}=0.656 min, [M+H]⁺=1020.6. ¹H NMR (400 MHZ, CHLOROFORM-d) δ=10.71-10.12 (m, 1H), 8.65-8.48 (m, 1H), 8.27 (s, 1H), 7.87-7.57 (m, 3H), 7.40-7.27 (m, 4H), 7.20-7.11 (m, 3H), 7.01-6.95 (m, 1H), 6.49-6.38 (m, 3H), 5.02 (m, 1H), 4.74-4.49 (m, 3H), 4.06-3.95 (m, 2H), 3.88-3.57 (m, 14H), 3.54-3.33 (m, 4H), 3.32-3.17 (m, 2H), 3.04-2.90 (m, 1H), 2.74-2.56 (m, 2H), 2.54 (m, 2H), 2.39-2.10 (m, 8H), 2.05-1.98 (m, 4H), 1.87-1.76 (m, 3H), 1.75-1.64 (m, 1H), 1.62-1.47 (m, 3H), 1.46-1.35 (m, 3H). SFC: R^{t}=1.756, 2.230.

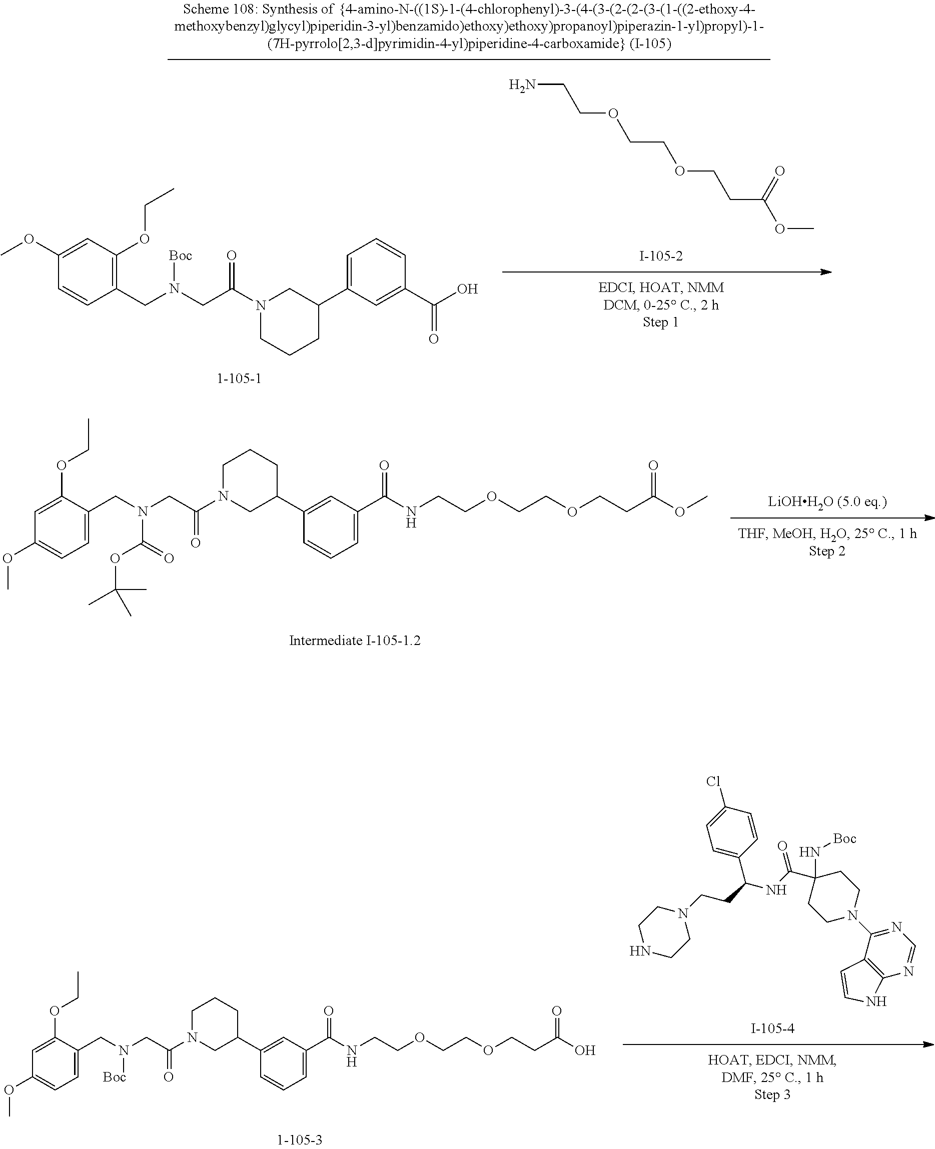 [SMILES: CCOc1cc(OC)ccc1CN(CC(=O)N1CCCC(c2cccc(C(=O)O)c2)C1)C(=O)OC(C)(C)C ; COC(=O)CCOCCOCCN ; CCOc1cc(OC)ccc1CN(CC(=O)N1CCCC(c2cccc(C(=O)NCCOCCOCCC(=O)OC)c2)C1)C(=O)OC(C)(C)C ; CCOc1cc(OC)ccc1CN(CC(=O)N1CCCC(c2cccc(C(=O)NCCOCCOCCC(=O)O)c2)C1)C(=O)OC(C)(C)C ; CC(C)(C)OC(=O)NC1(C(=O)N[C@@H](CCN2CCNCC2)c2ccc(Cl)cc2)CCN(c2ncnc3[nH]ccc23)CC1]

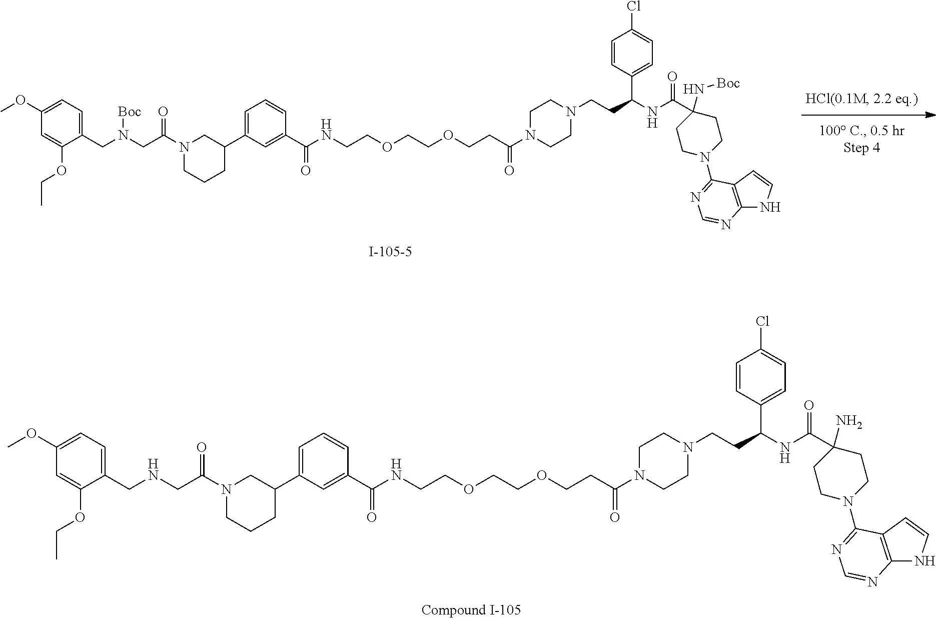 [SMILES: CCOc1cc(OC)ccc1CN(CC(=O)N1CCCC(c2cccc(C(=O)NCCOCCOCCC(=O)N3CCN(CC[C@H](NC(=O)C4(NC(=O)OC(C)(C)C)CCN(c5ncnc6[nH]ccc56)CC4)c4ccc(Cl)cc4)CC3)c2)C1)C(=O)OC(C)(C)C ; CCOc1cc(OC)ccc1CNCC(=O)N1CCCC(c2cccc(C(=O)NCCOCCOCCC(=O)N3CCN(CC[C@H](NC(=O)C4(N)CCN(c5ncnc6[nH]ccc56)CC4)c4ccc(Cl)cc4)CC3)c2)C1]

### Step 1: Synthesis of I-105-1.2

To a solution of I-105-1 (1 g, 1.90 mmol, 1 eq) and I-105-2 (864.70 mg, 3.80 mmol, 2 eq, HCl) in DCM (10 mL) was added EDCI (1.09 g, 5.70 mmol, 3 eq), HOAt (387.69 mg, 2.85 mmol, 1.5 eq). The resulting mixture was cooled to 0° C. via ice-bath, then NMM (1.92 g, 18.99 mmol, 2.09 mL, 10 eq) was introduced into the system drop-wisely. The mixture was mixture was then allowed to reach 25° C., and stirred for 2 hrs. The reaction mixture was quenched by water (50 mL) at under ice-bath, and extracted with DCM 60 mL (20 mL*3). The combined organic layers were washed with brine (30 mL*2) and dried over anhydrous Na₂SO₄, then filtered and concentrated under reduced pressure to give the crude. The crude was purified by Flash Silica-gel chromatography (ISCO®; 20 g SepaFlash® Silica Flash Column, Eluent of 95-100% EA/PE) and the eluent was concentrated to give I-105-1.2 (0.6 g, 852.17 μmol, 44.88% yield, 99.395% purity) as colorless oil. LCMS (Method E): R^{t}=0.570 min, [M+H]⁺=700.5. ¹H NMR (400 MHZ, CHLOROFORM-d) δ=7.79-7.55 (m, 2H), 7.44-7.28 (m, 2H), 7.25-7.06 (m, 1H), 6.94-6.78 (m, 1H), 6.53-6.33 (m, 2H), 4.76-4.65 (m, 1H), 4.62-4.35 (m, 2H), 4.05-3.92 (m, 3H), 3.84-3.72 (m, 6H), 3.69-3.59 (m, 10H), 3.10-2.92 (m, 1H), 2.80-2.49 (m, 4H), 2.13-2.05 (m, 1H), 1.96-1.87 (m, 2H), 1.86-1.78 (m, 1H), 1.77-1.66 (m, 1H), 1.64-1.55 (m, 1H), 1.53-1.32 (m, 12H).

### Step 2: Synthesis of I-105-3

To a solution of I-105-1.2 (0.20 g, 285.78 μmol, 1 eq) in THF (0.5 mL), MeOH (0.5 mL) and H₂O (0.5 mL) was added LiOH·H₂O (59.96 mg, 1.43 mmol, 5 eq). The mixture was stirred at 25° C. for 1 hr. The reaction mixture was quenched with HCl (10 ml, 1M, Aqueous), then extracted with EA (5 mL*3). The combined organic layer was dried by anhydrous Na₂SO₄ and concentrated under reduced pressure to afford I-105-3 (180 mg, 249.34 μmol, 87.25% yield, 95% purity) as colorless oil. LCMS (Method E): R^{t}=0.536 min, [M+H]⁺=686.4.

### Step 3: Synthesis of I-105-5

To a solution of I-105-3 (0.18 g, 262.47 μmol, 1.1 eq) in DMF (2 mL), was added EDCI (137.22 mg, 715.82 μmol, 3 eq), HOAt (48.71 mg, 357.91 μmol, 1.5 eq) and I-105-4 (142.48 mg, 238.61 μmol, 1 eq). Then NMM (241.35 mg, 2.39 mmol, 262.34 μL, 10 eq) was introduced into the system drop-wisely, the resulting mixture was allowed to stirred at 25° C. for 1 hr. The reaction mixture was purified by revers-phase HPLC (FA condition, Combine flash (26.8*125 mm, 80 g of XB—C18, 20-40 μm, 120 Å) Mobile phase: A for H₂O (0.1% FA v/v) and B for acetonitrile; Gradient: B 60%-70% in 30 min: Flow rate: 40 ml/min; Column temperature: R.T. Wavelength: 220 nm/254 nm), the eluent was lyophilized to give I-105-5 (0.109 g, 86.17 μmol, 36.11% yield, 100% purity) as an off-white solid. LCMS (Method E): R^{t}=0.652 min, [M+Na]⁺=1286.4.

### Step 4: Synthesis of I-105

I-105-5 (0.101 g, 79.85 μmol, 1 eq) was added in HCl (0.1 M, 1.76 mL, 2.2 eq). The mixture was heated to 100° C., and stirred for 0.5 hrs. The reaction mixture was lyophilized directly to afford the crude. The crude was purified by prep-HPLC (Basic condition, column: column: Waters Xbridge 150*25 mm*5 um; mobile phase: [water (ammonia hydroxide v/v)-ACN]; gradient: 30%-60% B) then lyophilized to afford I-105 (0.0218 g, 20.48 μmol, 25.64% yield, 100% purity) as a white solid. LCMS (Method E): R^{t}=0.637 min, [M+H]⁺=1064.5. SFC: R^{t}=1.965 min, 2.394 min. ¹H NMR (400 MHz, CHLOROFORM-d) δ=10.15-9.60 (m, 1H), 8.69-8.51 (m, 1H), 8.25 (d, J=6.5 Hz, 1H), 7.84-7.54 (m, 3H), 7.32-7.28 (m, 3H), 7.20-7.12 (m, 3H), 7.04-6.99 (m, 1H), 6.51-6.38 (m, 3H), 5.09-4.95 (m, 1H), 4.77-4.54 (m, 3H), 4.09-3.93 (m, 2H), 3.89-3.58 (m, 19H), 3.56-3.44 (m, 2H), 3.42-3.31 (m, 2H), 3.11-2.93 (m, 3H), 2.75-2.47 (m, 2H), 2.40-2.14 (m, 9H), 2.12-1.94 (m, 3H), 1.86-1.77 (m, 3H), 1.61-1.54 (m, 3H), 1.46-1.38 (m, 3H).

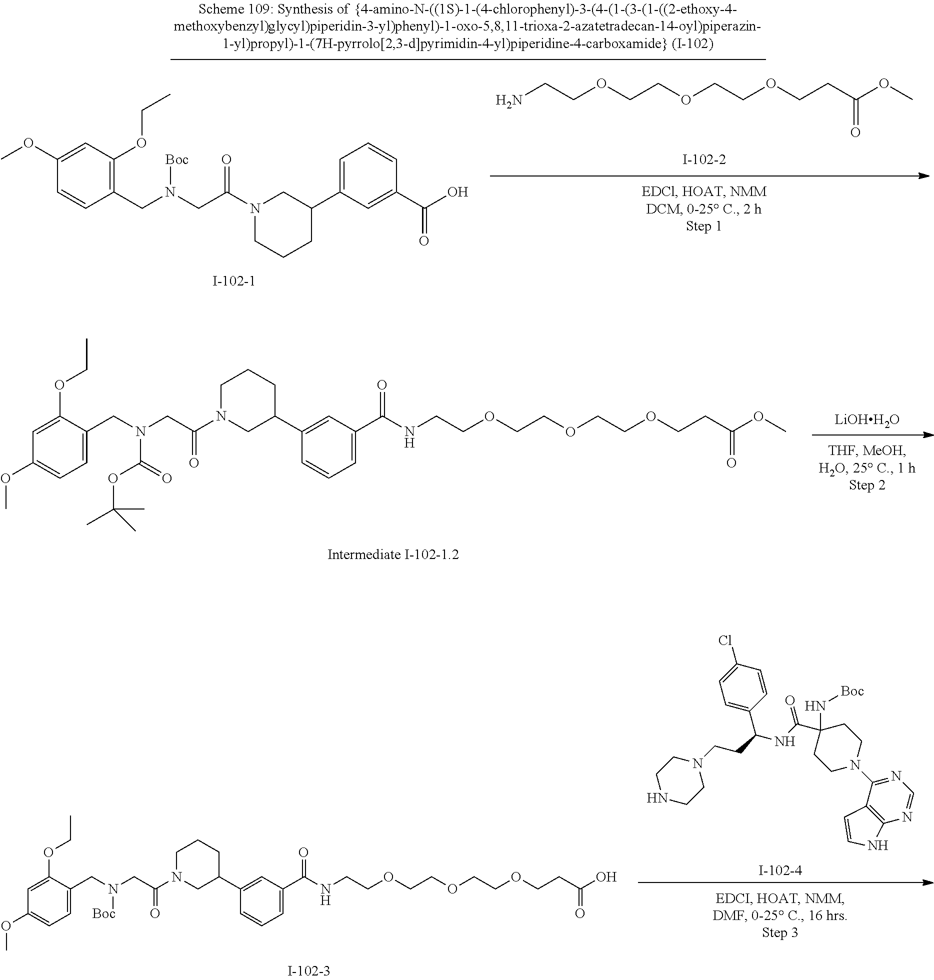

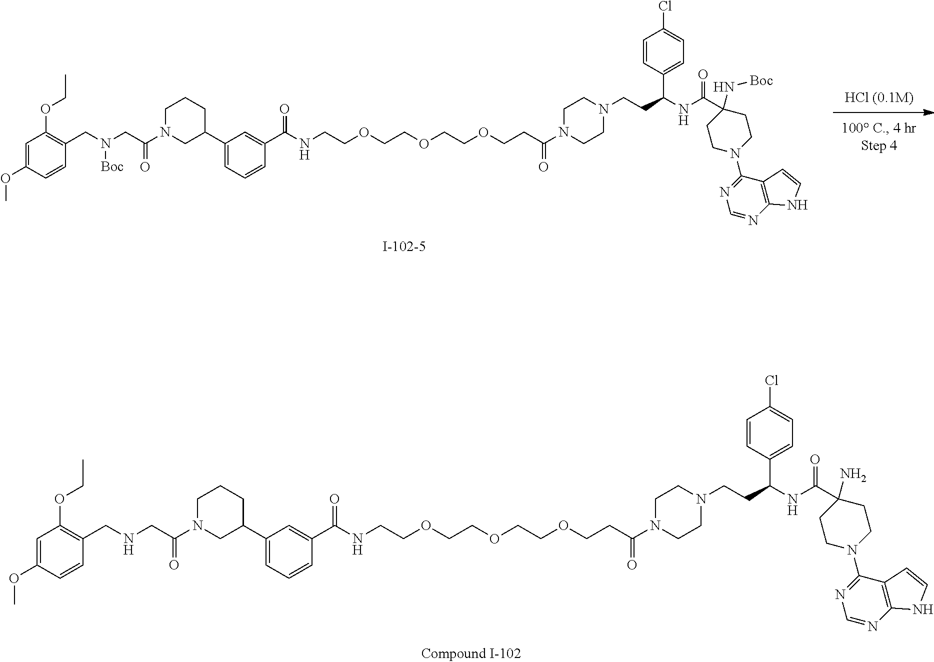 [SMILES: CCOc1cc(OC)ccc1CN(CC(=O)N1CCCC(c2cccc(C(=O)NCCOCCOCCOCCC(=O)N3CCN(CC[C@H](NC(=O)C4(NC(=O)OC(C)(C)C)CCN(c5ncnc6[nH]ccc56)CC4)c4ccc(Cl)cc4)CC3)c2)C1)C(=O)OC(C)(C)C ; "HCl (0.1M)" ; CCOc1cc(OC)ccc1CNCC(=O)N1CCCC(c2cccc(C(=O)NCCOCCOCCOCCC(=O)N3CCN(CC[C@H](NC(=O)C4(N)CCN(c5ncnc6[nH]ccc56)CC4)c4ccc(Cl)cc4)CC3)c2)C1]

### Step 1: Synthesis of I-102-2.1

To a solution of I-102-1 (1 g, 1.90 mmol, 1 eq), I-102-2 (1.03 g, 3.80 mmol, 2 eq, HCl), EDCI (1.09 g, 5.70 mmol, 3 eq), HOAt (387.69 mg, 2.85 mmol, 1.5 eq) in DCM (10 mL) was added NMM (1.92 g, 18.99 mmol, 2.09 mL, 10 eq). The mixture was stirred at 0-25° C. for 2 hr. The reaction mixture was quenched by addition H₂O 10 mL at 25° C., and extracted with DCM 15 mL (5 mL*3). The combined organic layers were dried over Na₂SO₄, filtered and concentrated under reduced pressure to give a residue. The residue was purified by flash silica gel chromatography (ISCO®; 12 g SepaFlash® Silica Flash Column, Eluent of 70˜100% Ethyl acetate/Petroleum ether gradient @ 30 mL/min) to give I-102-2.1 (1.1 g, 1.47 mmol, 77.29% yield, 99.254% purity) as colorless oil. LCMS (Method E): R^{t}=0.591 min, [M+H]⁺=744.5. ¹H NMR (400 MHz, DMSO-d₆) δ=8.59-8.43 (m, 1H), 7.79-7.66 (m, 2H), 7.48-7.34 (m, 2H), 7.14-7.00 (m, 1H), 6.57-6.42 (m, 2H), 4.52-4.38 (m, 1H), 4.36-4.15 (m, 2H), 4.03-3.86 (m, 4H), 3.84-3.67 (m, 4H), 3.65-3.56 (m, 5H), 3.55-3.44 (m, 10H), 3.44-3.37 (m, 2H), 3.15-2.93 (m, 1H), 2.76-2.53 (m, 4H), 1.97-1.87 (m, 1H), 1.85-1.65 (m, 2H), 1.49-1.26 (m, 13H).

### Step 2: Synthesis of I-102-3

To a solution of I-102-2.1 (160 mg, 215.09 μmol, 1 eq) in THF (0.5 mL), MeOH (0.5 mL) H₂O (0.5 mL) was added LiOH·H₂O (45.13 mg, 1.08 mmol, 5 eq). The mixture was stirred at 25° C. for 1 hr. The reaction mixture was quenched by addition 1M HCl 5 mL at 0° C., and then diluted with H₂O 10 mL and extracted with EA 15 mL (5 mL*3). The combined organic layers were dried over Na₂SO₄, filtered and concentrated under reduced pressure to give I-102-3 (155 mg, 203.42 μmol, 94.58% yield, 95.787% purity) as colorless oil. LCMS (Method E): R=0.534 min, [M+H]⁺=730.4.

### Step 3: Synthesis of I-102-5

To a solution of I-102-3 (155 mg, 212.37 μmol, 1.1 eq), I-102-4 (115.29 mg, 193.06 μmol, 1 eq), EDCI (111.03 mg, 579.19 μmol, 3 eq), HOAt (39.42 mg, 289.60 μmol, 1.5 eq) in DMF (2 mL) was added NMM (195.28 mg, 1.93 mmol, 212.26 μL, 10 eq). The mixture was stirred at 0-25° C. for 16 hr. The mixture was purified by prep-HPLC (FA condition) to give I-102-5 (160 mg, yield 58.12%, purity 91.8%) as a light yellow solid. LCMS (Method G): R^{t}=0.729 min, [M+H]⁺=1308.8. ¹H NMR (400 MHZ, DMSO-d₆) δ=8.55-8.44 (m, 1H), 8.36-8.29 (m, 1H), 8.09 (s, 1H), 7.78-7.61 (m, 2H), 7.46-7.33 (m, 2H), 7.29 (s, 4H), 7.14 (d, J=3.6 Hz, 2H), 7.09-6.99 (m, 1H), 6.57 (d, J=3.6 Hz, 1H), 6.53-6.40 (m, 2H), 4.90-4.78 (m, 1H), 4.48-4.36 (m, 1H), 4.34-4.13 (m, 4H), 4.09-3.82 (m, 5H), 3.81-3.65 (m, 5H), 3.53-3.41 (m, 15H), 3.14-2.91 (m, 1H), 2.78-2.54 (m, 4H), 2.41-2.10 (m, 7H), 2.05-1.61 (m, 10H), 1.57-1.12 (m, 24H).

### Step 4: Synthesis of I-102

The mixture of I-102-5 (140 mg, 106.95 μmol, 1 eq) in dioxane (2 mL) was added HCl (0.1 M, 4.28 mL, 4 eq). The mixture was stirred at 100° C. for 4 hr. The mixture was lyophilized to get the crude product. The residue was purified by prep-HPLC (column: Waters Xbridge 150*25 mm*5 um; mobile phase: [water (ammonia hydroxide v/v)-ACN]; gradient: 28%-58% B over 10 min). The desired peak was lyophilized to give I-102 (42.59 mg, 38.41 μmol, 35.92% yield, 100% purity) as an off-white solid. LCMS (Method G): R^{t}=0.670 min, [M+H]⁺=1108.7. SFC: R^{t}=2.025, 2.491 min, H10. ¹H NMR (400 MHZ, CHLOROFORM-d) δ=10.00-9.71 (m, 1H), 8.73-8.57 (m, 1H), 8.34-8.23 (m, 1H), 7.80-7.60 (m, 2H), 7.40-7.28 (m, 5H), 7.20-7.11 (m, 3H), 7.11-7.04 (m, 1H), 6.52-6.37 (m, 3H), 5.08-4.99 (m, 1H), 4.75-4.55 (m, 3H), 4.09-3.95 (m, 2H), 3.86-3.72 (m, 8H), 3.72-3.60 (m, 13H), 3.53-3.37 (m, 4H), 3.23-2.94 (m, 3H), 2.75-2.51 (m, 2H), 2.46-1.99 (m, 12H), 1.85-1.78 (m, 4H), 1.64-1.53 (m, 3H), 1.46-1.38 (m, 3H).

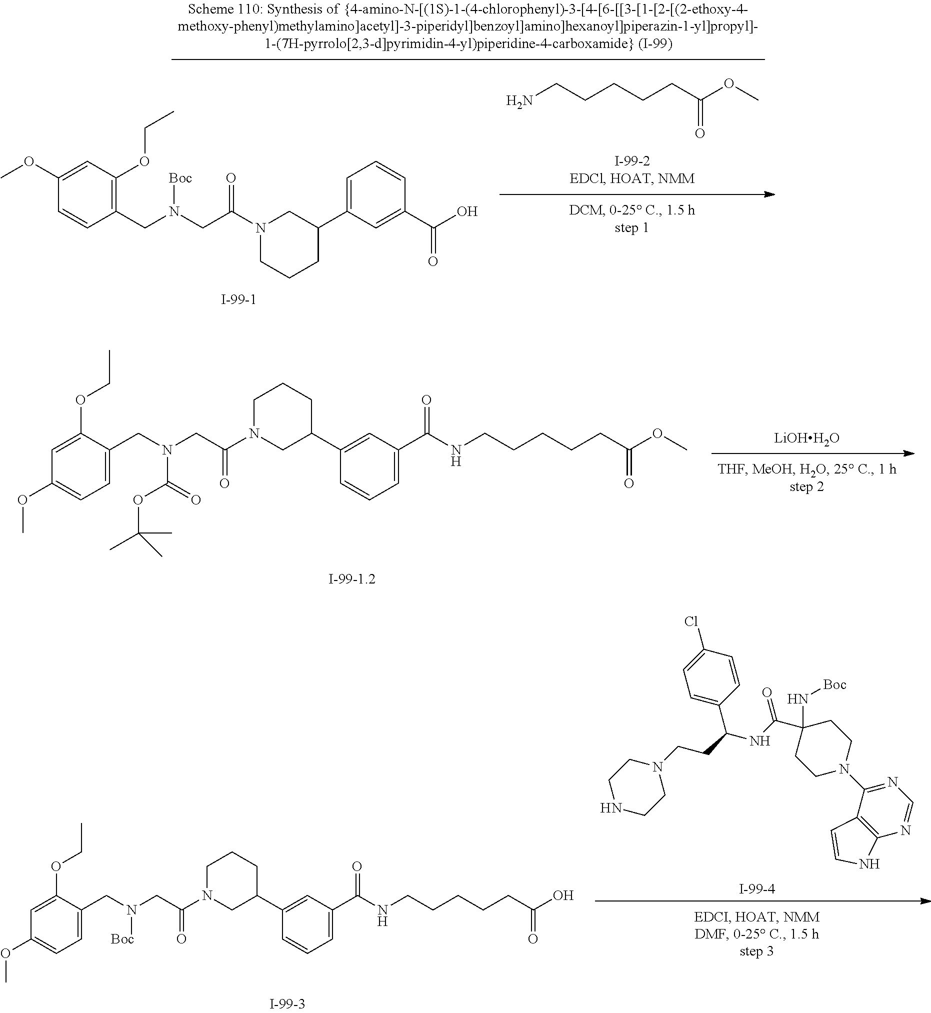

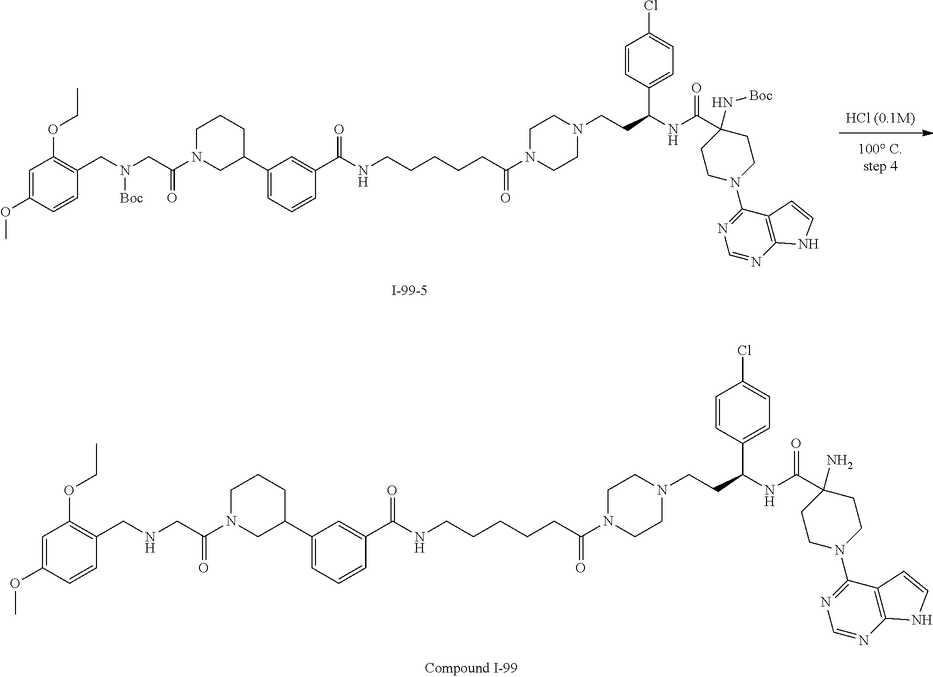 [SMILES: CCOc1cc(OC)ccc1CN(CC(=O)N1CCCC(c2cccc(C(=O)NCCCCCC(=O)N3CCN(CC[C@H](NC(=O)C4(NC(=O)OC(C)(C)C)CCN(c5ncnc6[nH]ccc56)CC4)c4ccc(Cl)cc4)CC3)c2)C1)C(=O)OC(C)(C)C ; "HCl (0.1M)" ; CCOc1cc(OC)ccc1CNCC(=O)N1CCCC(c2cccc(C(=O)NCCCCCC(=O)N3CCN(CC[C@H](NC(=O)C4(N)CCN(c5ncnc6[nH]ccc56)CC4)c4ccc(Cl)cc4)CC3)c2)C1]

### Step 1: Synthesis of I-99-1.2

To a solution of I-99-1 (0.1 g, 189.89 μmol, 1 eq) in DCM (1 mL) was added EDCI (109.21 mg, 569.67 μmol, 3 eq), NMM (96.03 mg, 949.45 μmol, 104.38 UL, 5 eq), and HOAt (38.77 mg, 284.84 μmol, 39.85 ML, 1.5 eq). Then I-99-2 (41.39 mg, 227.87 μmol, 1.2 eq, HCl) was added. The reaction mixture was stirred at 25° C. for 1.5 hr. The reaction mixture was quenched by addition H₂O 3 mL at 25° C., then extracted with EA 6 mL (2 mL*3). The combined organic layers were dried over Na₂SO₄, filtered and concentrated under reduced pressure to give a residue. The crude was purified by Prep-HPLC (column: Phenomenex luna C18 150*25 mm*10 um; mobile phase: [water (FA)-ACN]; gradient: 50%-80% B over 10 min). After prep-HPLC purification, the eluent was concentrated to remove organic solvents and then lyophilized to give I-99-1.2 (60 mg, 91.40 μmol, 48.13% yield, 99.6% purity) as off-white solid. LCMS (Method E): R^{t}=0.604 min, (M+H)=654.4. ¹H NMR (400 MHZ, DMSO-d₆) δ=8.48-8.37 (m, 1H), 7.77-7.64 (m, 2H), 7.46-7.33 (m, 2H), 7.08-7.02 (m, 1H), 6.57-6.40 (m, 2H), 4.50-4.39 (m, 1H), 4.35-4.17 (m, 2H), 4.10-3.88 (m, 4H), 3.72 (br d, J=9.6 Hz, 4H), 3.57 (s, 3H), 3.26-3.20 (m, 2H), 3.14-2.94 (m, 1H), 2.74-2.55 (m, 2H), 2.32-2.28 (m, 2H), 1.93 (br d, J=9.6 Hz, 1H), 1.82-1.67 (m, 2H), 1.62-1.42 (m, 5H), 1.40-1.25 (m, 14H).

### Step 2: Synthesis of I-99-3

To a solution of I-99-1.2 (1.1 g, 1.68 mmol, 1 eq) in THF (3 mL) MeOH (3 mL) H2O (3 mL) was added LiOH·H2O (353.01 mg, 8.41 mmol, 5 eq). The mixture was stirred at 25° C. for 1 hr. The mixture was adjusted to pH=3 with HCl solution and extracted with EA (15 mL*3), the organic layers were dried over Na₂SO₄, filtered, and concentrated to give I-99-2 (1 g, 1.56 mmol, 92.90% yield, 100% purity) as a white solid. LCMS (Method E): R^{t}=0.562 min, [M+H]⁺=640.5. ¹H NMR (400 MHZ, DMSO-d₆) δ=11.97 (s, 1H), 8.53-8.32 (m, 1H), 7.79-7.65 (m, 2H), 7.45-7.34 (m, 2H), 7.14-7.00 (m, 1H), 6.57-6.42 (m, 2H), 4.53-4.38 (m, 1H), 4.34-4.16 (m, 2H), 4.05-3.90 (m, 4H), 3.75-3.70 (m, 3H), 3.24 (q, J=6.4 Hz, 2H), 3.14-2.95 (m, 1H), 2.78-2.56 (m, 2H), 2.21 (t, J=7.2 Hz, 2H), 1.94 (br s, 1H), 1.83-1.68 (m, 2H), 1.62-1.21 (m, 20H).

### Step 3: Synthesis of I-99-5

To a solution of I-99-3 (117.85 mg, 184.21 μmol, 1.1 eq) in DMF (1 mL) was added EDCI (96.31 mg, 502.39 μmol, 3 eq), NMM (84.69 mg, 837.31 μmol, 92.06 μL, 5 eq), and HOAt (34.19 mg, 251.19 μmol, 35.14 μL, 1.5 eq). Then I-99-4 (0.1 g, 167.46 μmol, 1 eq) was added. The reaction mixture was stirred at 25° C. for 1.5 hr. The mixture was purified by reversed phase (0.1% FA). After purification, the eluent was concentrated to remove organic solvents and then lyophilized to give I-99-5 (160 mg, 120.76 μmol, 72.11% yield, 92% purity) as white solid. LCMS (Method G): R^{t}=0.777 min, [M+H]⁺=525.4. 1H NMR (400 MHZ, DMSO-d₆) δ=11.76-11.59 (m, 1H), 8.48-8.34 (m, 2H), 8.33-8.27 (m, 1H), 8.12 (s, 1H), 7.77-7.66 (m, 2H), 7.44-7.37 (m, 2H), 7.32 (s, 4H), 7.24-7.13 (m, 2H), 7.10-7.03 (m, 1H), 6.63-6.57 (m, 1H), 6.55-6.46 (m, 2H), 4.93-4.81 (m, 1H), 4.51-4.39 (m, 1H), 4.27-4.18 (m, 3H), 4.09-3.96 (m, 4H), 3.76-3.69 (m, 4H), 3.46 (br s, 4H), 3.10-2.95 (m, 2H), 2.70-2.59 (m, 2H), 2.38-2.17 (m, 9H), 2.00-1.90 (m, 5H), 1.81-1.69 (m, 3H), 1.57-1.47 (m, 5H), 1.41 (br s, 8H), 1.37 (br d, J=5.6 Hz, 13H), 1.31 (m, 5H).

### Step 4: Synthesis of 1-99

A suspension of I-99-5 (0.14 g, 114.86 μmol, 1 eq) in HCl (0.1 M, 2.30 mL, 2 eq), dioxane (0.3 mL) was stirred at 100° C. for 0.5 h. The mixture was added NaHCO₃ to adjust pH=9˜10, and then lyophilized to give residue. The crude was purified by Prep-HPLC (column: Waters Xbridge 150*25 mm*5 um; mobile phase: [water (ammonia hydroxide v/v)-ACN]; gradient: 30%-60% B over 15 min). After prep-HPLC purification, the eluent was concentrated to remove organic solvents and then lyophilized to give I-99 (14.72 mg, 14.41 μmol, 12.54% yield, 99.7% purity) as white solid. LCMS (Method G): R^{t}=0.673 min, [M+H]⁺=1018.7. SFC: R^{t}=1.814 min, 2.313 min. ¹H NMR (400 MHZ, CHLOROFORM-d) δ=8.76-8.61 (m, 1H), 8.30-8.23 (m, 1H), 7.79-7.64 (m, 2H), 7.41-7.27 (m, 5H), 7.21-7.16 (m, 3H), 7.03 (d, J=3.6 Hz, 1H), 6.48 (d, J=3.6 Hz, 1H), 6.43 (br d, J=10.8 Hz, 2H), 5.31 (s, 1H), 5.08-4.98 (m, 1H), 4.67-4.49 (m, 3H), 4.04-4.02 (m, 2H), 3.93-3.85 (m, 2H), 3.79 (d, J=6.4 Hz, 4H), 3.68-3.57 (m, 4H), 3.54-3.46 (m, 4H), 3.43-3.33 (m, 2H), 3.04-2.96 (m, 1H), 2.74-2.59 (m, 2H), 2.45-2.18 (m, 12H), 2.10-1.95 (m, 4H), 1.70-1.60 (m, 8H), 1.47-1.39 (m, 6H).

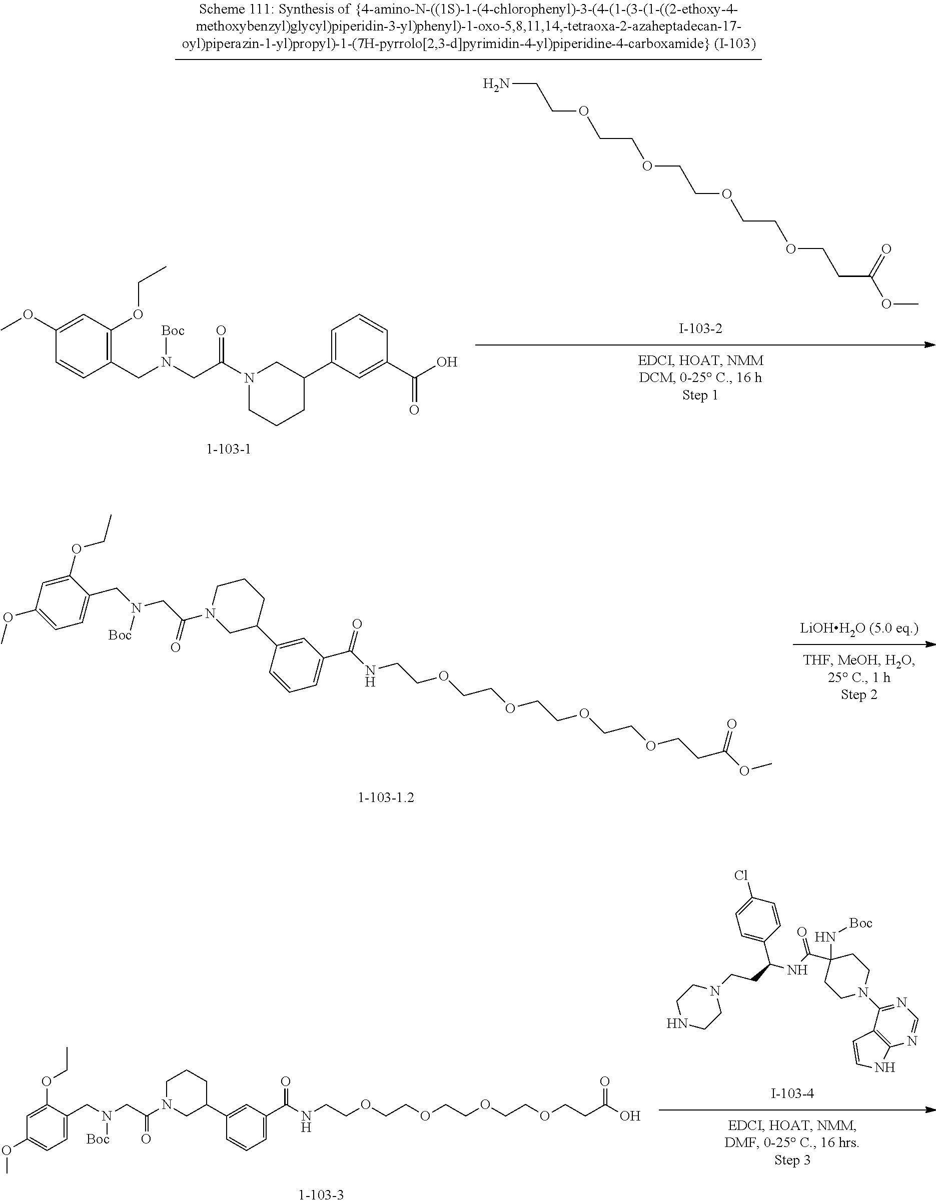

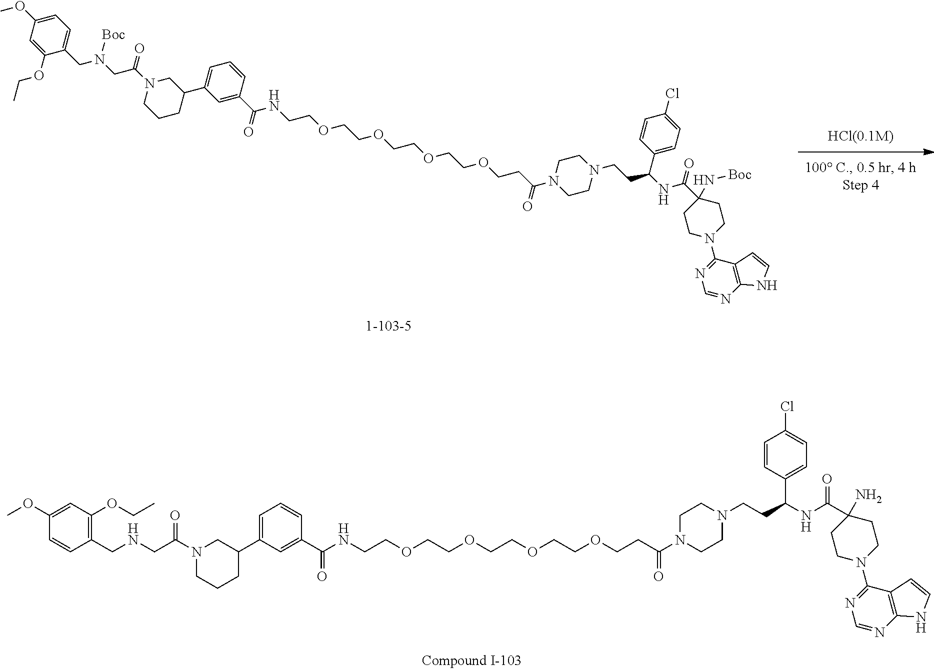 [SMILES: CCOc1cc(OC)ccc1CN(CC(=O)N1CCCC(c2cccc(C(=O)NCCOCCOCCOCCOCCC(=O)N3CCN(CC[C@H](NC(=O)C4(NC(=O)OC(C)(C)C)CCN(c5ncnc6[nH]ccc56)CC4)c4ccc(Cl)cc4)CC3)c2)C1)C(=O)OC(C)(C)C ; CCOc1cc(OC)ccc1CNCC(=O)N1CCCC(c2cccc(C(=O)NCCOCCOCCOCCOCCC(=O)N3CCN(CC[C@H](NC(=O)C4(N)CCN(c5ncnc6[nH]ccc56)CC4)c4ccc(Cl)cc4)CC3)c2)C1]

### Step 1: Synthesis of I-103-1.2

To a solution of I-103-1 (1 g, 1.90 mmol, 1 eq), I-103-2 (1.06 g, 3.36 mmol, 1.77 eq, HCl), EDCI (1.09 g, 5.70 mmol, 3 eq) and HOAt (387.69 mg, 2.85 mmol, 1.5 eq) in DCM (10 mL) was added NMM (1.92 g, 18.99 mmol, 2.09 mL, 10 eq) at 0° C. The mixture was stirred at 0-25° C. for 16 hr. The reaction mixture was quenched by addition H₂O 10 mL at 25° C., and extracted with DCM 15 mL (5 mL*3). The combined organic layers were dried over Na₂SO₄, filtered and concentrated under reduced pressure to give a residue. The residue was purified by flash silica gel chromatography (ISCO®; 12 g SepaFlash® Silica Flash Column, Eluent of 70˜100% Ethylacetate/Petroleum ether gradient @ 30 mL/min) to afford I-103-1.2 (1.1 g, 1.38 mmol, 72.65% yield, 98.814% purity) as yellow oil. LCMS (Method E): R^{t}=0.599 min, [M+H]⁺=788.5. ¹H NMR (400 MHZ, DMSO-d₆) δ=8.59-8.47 (m, 1H), 7.80-7.65 (m, 2H), 7.48-7.34 (m, 2H), 7.12-6.99 (m, 1H), 6.56-6.42 (m, 2H), 4.56-4.38 (m, 1H), 4.37-4.15 (m, 2H), 4.03-3.87 (m, 4H), 3.85-3.67 (m, 4H), 3.64-3.56 (m, 5H), 3.52 (br s, 6H), 3.50-3.44 (m, 8H), 3.43-3.38 (m, 2H), 3.14-2.93 (m, 1H), 2.79-2.53 (m, 4H), 1.96-1.86 (m, 1H), 1.85-1.66 (m, 2H), 1.53-1.26 (m, 13H).

### Step 2: Synthesis of I-103-3

To a solution of I-103-1.2 (170 mg, 215.75 μmol, 1 eq) in THF (0.5 mL), MeOH (0.5 mL) and H₂O (0.5 mL) was added LiOH·H₂O (45.27 mg, 1.08 mmol, 5 eq). The mixture was stirred at 25° C. for 1 hr. The reaction mixture was quenched by addition 1M HCl 5 mL at 0° C., and then diluted with H₂O 10 mL and extracted with EA 15 mL (5 mL*3). The combined organic layers were dried over Na₂SO₄, filtered and concentrated under reduced pressure to give I-103-3 (155 mg, 189.30 μmol, 87.74% yield, 94.516% purity) as colorless oil. LCMS (Method E): R^{t}=0.535 min, [M+H]⁺=774.4.

### Step 3: Synthesis of I-103-5

To a solution of I-103-3 (155 mg, 200.28 μmol, 1.1 eq), I-103-4 (108.73 mg, 182.07 μmol, 1 eq), EDCI (104.71 mg, 546.22 μmol, 3 eq), HOAt (37.17 mg, 273.11 μmol, 1.5 eq) in DMF (2 mL) was added NMM (184.16 mg, 1.82 mmol, 200.18 μL, 10 eq). The mixture was stirred at 0-25° C. for 16 hr. The mixture was used into purification without workup. The residue was purified by prep-HPLC (FA condition) to give I-103-5 (160 mg, yield 58.72%, purity 90.415%) as a light yellow solid. LCMS (Method G): R^{t}=0.721 min, [M+H]⁺=1352.8. ¹H NMR (400 MHZ, DMSO-d₆) δ=8.55-8.43 (m, 1H), 8.36-8.29 (m, 1H), 8.10 (s, 1H), 7.77-7.64 (m, 2H), 7.44-7.33 (m, 2H), 7.29 (s, 4H), 7.20-7.11 (m, 2H), 7.09-7.00 (m, 1H), 6.57 (d, J=3.6 Hz, 1H), 6.53-6.41 (m, 2H), 4.90-4.79 (m, 1H), 4.46-4.36 (m, 1H), 4.34-4.11 (m, 4H), 4.09-3.85 (m, 5H), 3.82-3.67 (m, 5H), 3.60-3.56 (m, 13H), 3.45-3.43 (m, 5H), 3.13-2.90 (m, 1H), 2.77-2.53 (m, 4H), 2.40-2.14 (m, 7H), 2.06-1.62 (m, 10H), 1.50-1.22 (m, 24H),

### Step 4: Synthesis of I-103

The mixture of I-103-5 (140 mg, 103.47 μmol, 1 eq) in dioxane (2 mL) was added HCl (0.1 M, 4.14 mL, 4 eq). The mixture was stirred at 100° C. for 4 hr. The mixture was lyophilized to get the crude product. The residue was purified by prep-HPLC (column: Waters Xbridge 150*25 mm*5 um; mobile phase: [water (ammonia hydroxide v/v)-ACN]; gradient: 28%-58% B over 10 min). The desired peak was lyophilized to give I-103 (42.63 mg, 36.98 μmol, 35.74% yield, 100% purity) as a yellow solid. LCMS (Method G): R^{t}=0.666 min, [M+H]⁺=1152.7. SFC: R^{t}=2.168, 2.695 min, _H10. ¹H NMR (400 MHZ, CHLOROFORM-d) δ=10.52-10.13 (m, 1H), 8.73-8.61 (m, 1H), 8.35-8.19 (m, 1H), 7.81-7.65 (m, 2H), 7.58-7.45 (m, 1H), 7.39-7.27 (m, 4H), 7.20-7.10 (m, 3H), 7.05 (d, J=3.3 Hz, 1H), 6.52-6.36 (m, 3H), 5.06-4.97 (m, 1H), 4.72-4.51 (m, 3H), 4.08-3.92 (m, 2H), 3.84-3.71 (m, 8H), 3.70-3.56 (m, 17H), 3.52-3.39 (m, 4H), 3.27-3.07 (m, 2H), 3.05-2.90 (m, 1H), 2.75-2.54 (m, 2H), 2.53-2.42 (m, 2H), 2.40-2.13 (m, 10H), 1.87-1.65 (m, 4H), 1.63-1.50 (m, 3H), 1.46-1.36 (m, 3H).

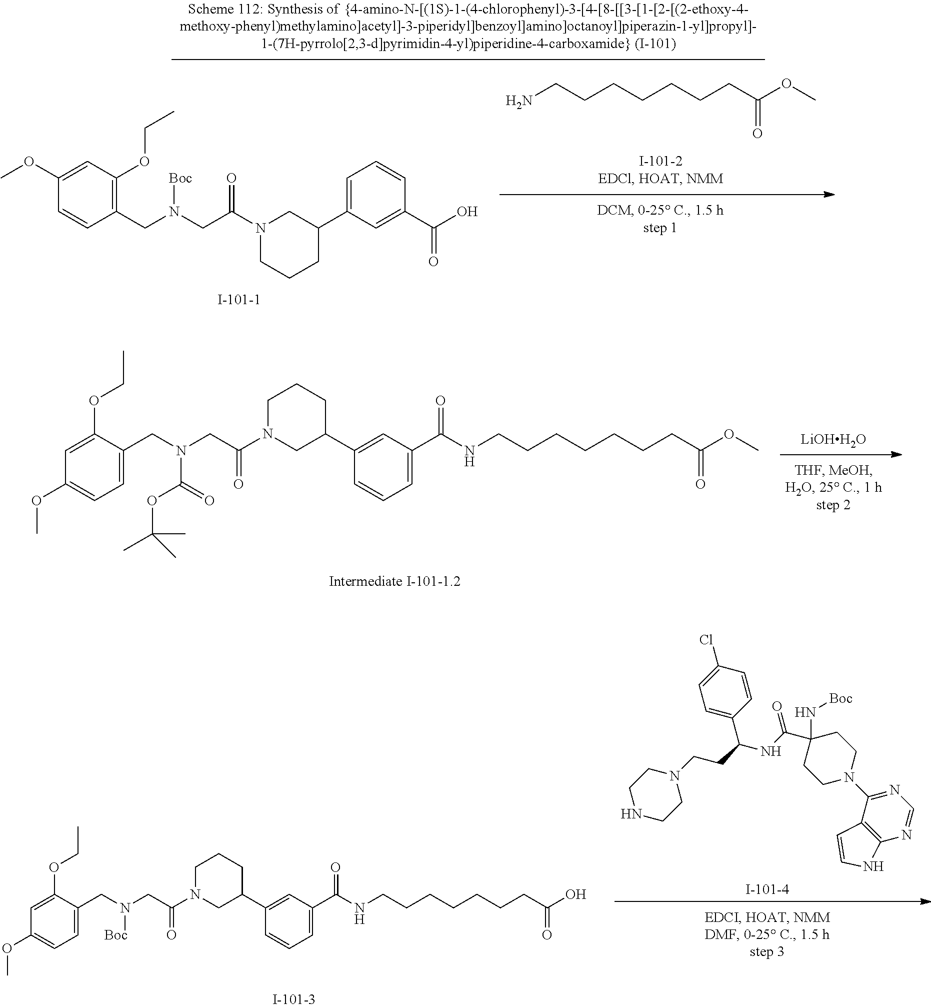

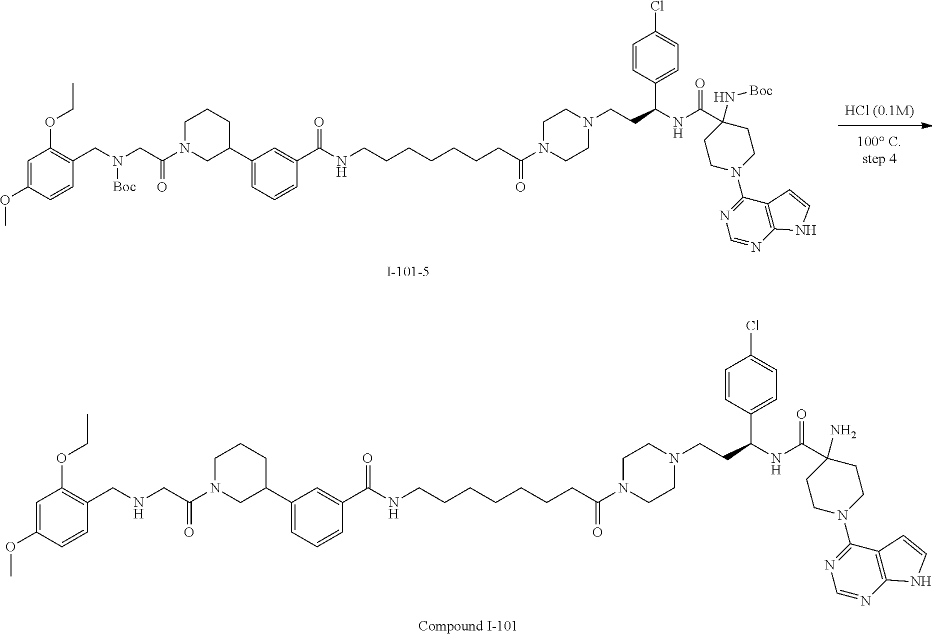 [SMILES: CCOc1cc(OC)ccc1CN(CC(=O)N1CCCC(c2cccc(C(=O)NCCCCCCCC(=O)N3CCN(CC[C@H](NC(=O)C4(NC(=O)OC(C)(C)C)CCN(c5ncnc6[nH]ccc56)CC4)c4ccc(Cl)cc4)CC3)c2)C1)C(=O)OC(C)(C)C ; "HCl (0.1M)" ; CCOc1cc(OC)ccc1CNCC(=O)N1CCCC(c2cccc(C(=O)NCCCCCCCC(=O)N3CCN(CC[C@H](NC(=O)C4(N)CCN(c5ncnc6[nH]ccc56)CC4)c4ccc(Cl)cc4)CC3)c2)C1]

### Step 1: Synthesis of I-101-1.2

To a solution of I-101-1 (0.1 g, 189.89 μmol, 1 eq) in DCM (1 mL) was added HOAt (38.77 mg, 284.84 μmol, 39.84 μL, 1.5 eq), EDCI (109.21 mg, 569.67 μmol, 3 eq), and NMM (96.03 mg, 949.45 μmol, 104.38 μL, 5 eq). Then I-101-2 (36.19 mg, 208.88 μmol, 1.1 eq) was added. The reaction mixture was stirred at 25° C. for 1.5 hr. The reaction mixture was quenched by addition H₂O 3 mL at 25° C., then extracted with EA 6 mL (2 mL*3). The combined organic layers were dried over Na₂SO₄, filtered and concentrated under reduced pressure to give a residue. The crude was purified by Prep-HPLC (column: Phenomenex luna C18 150*25 mm*10 um; mobile phase: [water (FA)-ACN]; gradient: 55%-85% B over 10 min). After prep-HPLC purification, the eluent was concentrated to remove organic solvents and then lyophilized to give I-101-1.2 (70 mg, 97.73 μmol, 51.47% yield, 95.2% purity) as off-white solid. LCMS (Method E): R^{t}=0.649 min, [M+H]⁺=682.5. ¹H NMR (400 MHZ, DMSO-d₆) δ=8.47-8.37 (m, 1H), 7.76-7.65 (m, 2H), 7.45-7.35 (m, 2H), 7.08-7.02 (m, 1H), 6.54-6.45 (m, 2H), 4.48-4.40 (m, 1H), 4.33-4.18 (m, 2H), 4.10-3.94 (m, 4H), 3.72 (br d, J=9.6 Hz, 4H), 3.57 (s, 3H), 3.26-3.20 (m, 2H), 3.13-2.97 (m, 1H), 2.72-2.57 (m, 2H), 2.35-2.24 (m, 2H), 1.93 (br d, J=10.4 Hz, 1H), 1.74 (br d, J=9.6 Hz, 2H), 1.57-1.42 (m, 5H), 1.38-1.26 (m, 18H).

### Step 2: Synthesis of I-101-3

To a solution of I-101-1.2 (1 g, 1.47 mmol, 1 eq) in THF (3 mL), MeOH (3 mL) and H₂O (3 mL) was added LiOH·H2O (307.72 mg, 7.33 mmol, 5 eq). The mixture was stirred at 25° C. for 1 hr. The mixture was adjusted to pH=3 with HCl solution and extracted with EA (15 mL*3), the organic layers were dried over Na₂SO₄, filtered and concentrated to give I-101-3 (970 mg, 1.45 mmol, 99.04% yield, 100% purity) as a white solid. LCMS (Method E): R^{t}=0.579 min, [M+H]⁺=668.5. ¹H NMR (400 MHZ, DMSO-d₆) δ=12.59-11.02 (m, 1H), 8.49-8.36 (m, 1H), 7.77-7.65 (m, 2H), 7.45-7.34 (m, 2H), 7.12-7.00 (m, 1H), 6.57-6.41 (m, 2H), 4.51-4.38 (m, 1H), 4.33-4.17 (m, 2H), 4.04-3.96 (m, 4H), 3.75-3.70 (m, 3H), 3.27-3.21 (m, 2H), 3.11-2.98 (m, 1H), 2.77-2.62 (m, 2H), 2.19 (t, J=7.2 Hz, 2H), 1.97-1.92 (m, 1H), 1.83-1.68 (m, 2H), 1.57-1.43 (m, 5H), 1.41-1.26 (m, 19H).

### Step 3: Synthesis of I-101-5

To a solution of I-101-3 (123.02 mg, 184.21 μmol, 1.1 eq) in DMF (1 mL) was added EDCI (96.31 mg, 502.39 μmol, 3 eq), NMM (84.69 mg, 837.31 μmol, 92.06 μL, 5 eq) and HOAt (34.19 mg, 251.19 μmol, 35.14 μL, 1.5 eq). Then I-101-4 (0.1 g, 167.46 μmol, 1 eq) was added. The reaction mixture was stirred at 25° C. for 1.5 hr. The mixture was purified by reversed phase (0.1% FA). After purification, the eluent was concentrated to remove organic solvents and then lyophilized to give I-101-5 (110 mg, 69.69 μmol, 41.61% yield, 79% purity) as white solid. LCMS (Method G): R^{t}=0.810 min, [M+H]⁺=525.4. 1H NMR (400 MHZ, DMSO-d₆) δ=11.66 (br s, 1H), 8.46-8.37 (m, 1H), 8.33-8.25 (m, 1H), 8.12 (s, 1H), 7.73-7.67 (m, 2H), 7.40 (br d, J=6.4 Hz, 2H), 7.31 (s, 4H), 7.22-7.12 (m, 2H), 7.06 (m, 1H), 6.59 (m, 1H), 6.54-6.45 (m, 2H), 4.91-4.82 (m, 1H), 4.48-4.39 (m, 1H), 4.26-4.17 (m, 3H), 4.10-3.96 (m, 4H), 3.72 (br d, J=10.8 Hz, 4H), 3.63-3.54 (m, 2H), 3.45 (br s, 5H), 3.14-2.94 (m, 2H), 2.71-2.59 (m, 2H), 2.31-2.17 (m, 7H), 2.02-1.88 (m, 6H), 1.84-1.68 (m, 4H), 1.55-1.46 (m, 4H), 1.41 (br s, 8H), 1.36 (br d, J=5.6 Hz, 12H), 1.29 (br s, 9H).

### Step 4: Synthesis of I-101

A suspension of I-101-5 (0.09 g, 72.18 μmol, 1 eq) in HCl (0.1 M, 1.44 mL, 2 eq) and dioxane (0.3 mL) was stirred at 100° C. for 0.5 h. The mixture was added NaHCO₃ to adjust pH=9˜10 and then lyophilized to give residue. The residue was purified by Prep-HPLC (column: Waters Xbridge 150*25 mm*5 um; mobile phase: [water (ammonia hydroxide v/v)-ACN]; gradient: 30%-60% B over 15 min). After prep-HPLC purification, the eluent was concentrated to remove organic solvents and then lyophilized to give I-101 (19.49 mg, 18.42 μmol, 25.51% yield, 98.9% purity) as white solid. LCMS (Method G): R^{t}=0.695 min, [M+H]⁺=1046.8. SFC: R^{t}=2.099 min, 2.878 min. ¹H NMR (400 MHZ, DMSO-d₆) δ=11.82-11.54 (m, 1H), 8.80-8.67 (m, 1H), 8.48-8.33 (m, 1H), 8.11 (s, 1H), 7.78-7.64 (m, 2H), 7.41-7.29 (m, 6H), 7.19-7.11 (m, 2H), 6.58-6.56 (m, 1H), 6.52-6.40 (m, 2H), 4.90-4.78 (m, 1H), 4.51-4.33 (m, 3H), 4.05-3.94 (m, 2H), 3.86-3.76 (m, 1H), 3.72 (d, J=6.8 Hz, 3H), 3.64-3.50 (m, 4H), 3.49-3.41 (m, 4H), 3.39-3.35 (m, 2H), 3.26-3.21 (m, 2H), 3.09-2.95 (m, 1H), 2.75-2.63 (m, 1H), 2.34-2.15 (m, 11H), 2.00-1.82 (m, 5H), 1.78-1.69 (m, 2H), 1.56-1.24 (m, 17H).

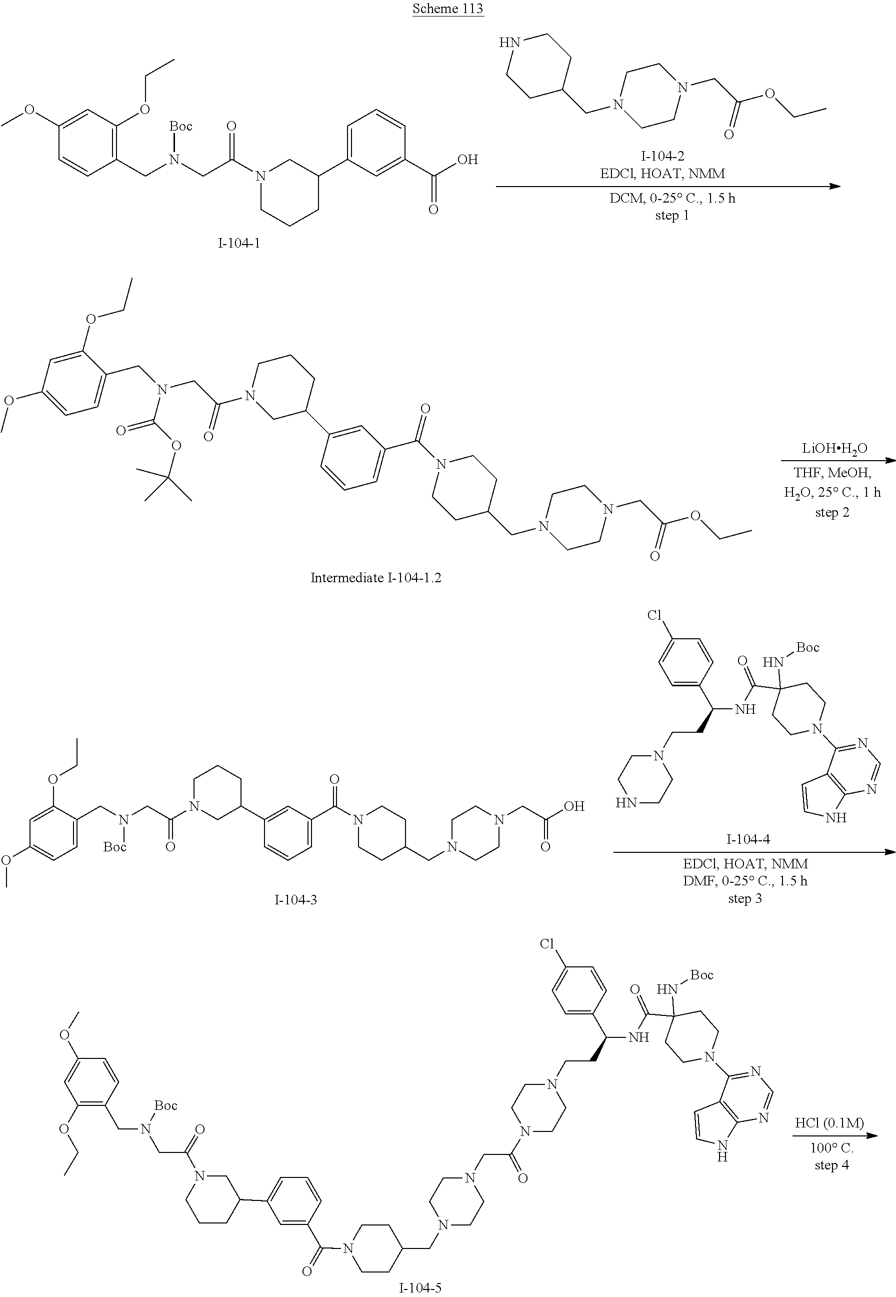

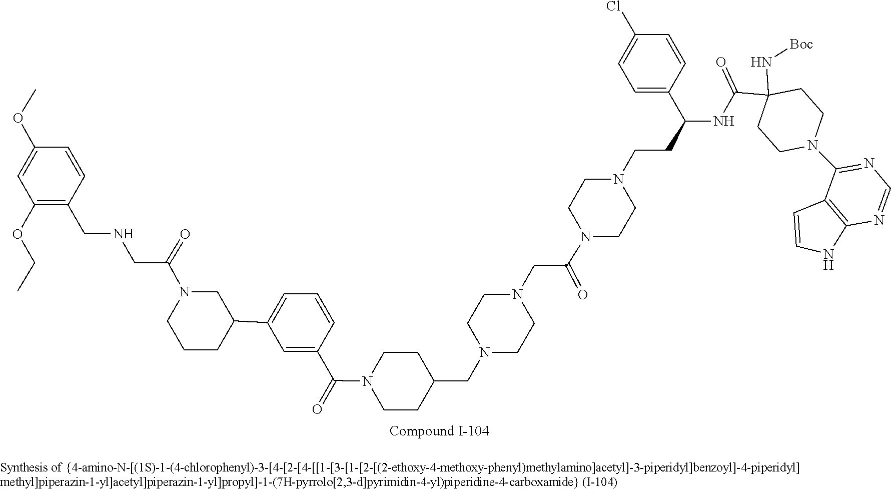 [SMILES: CCOc1cc(OC)ccc1CNCC(=O)N1CCCC(c2cccc(C(=O)N3CCC(CN4CCN(CC(=O)N5CCN(CC[C@H](NC(=O)C6(NC(=O)OC(C)(C)C)CCN(c7ncnc8[nH]ccc78)CC6)c6ccc(Cl)cc6)CC5)CC4)CC3)c2)C1]

### Step 1: Synthesis of I-104-1.2

To a solution of I-104-1 (0.1 g, 189.89 μmol, 1 eq) in DCM (1 mL) was added EDCI (109.21 mg, 569.67 μmol, 3 eq), NMM (96.03 mg, 949.45 μmol, 104.38 μL, 5 eq), and HOAt (38.77 mg, 284.84 μmol, 39.85 μL, 1.5 eq). Then I-104-2 (69.69 mg, 227.87 μmol, 1.2 eq, HCl) was added. The reaction mixture was stirred at 25° C. for 1.5 hr. The reaction mixture was quenched by addition H₂O 3 mL at 25° C., then extracted with EA 6 mL (2 mL*3). The combined organic layers were dried over Na₂SO₄, filtered and concentrated under reduced pressure to give a residue. The crude was purified by Prep-HPLC (column: C18 150×30 mm; mobile phase: [water (FA)-ACN]; gradient: 28%-58% B over 7 min). After prep-HPLC purification, the eluent was concentrated to remove organic solvents and then lyophilized to give product. I-104-1.2 (40 mg, 51.41 μmol, 27.08% yield, 100% purity) was obtained as white solid. LCMS (Method E): R^{t}=0.526 min, [M+H]⁺=778.5. ¹H NMR (400 MHZ, DMSO-d₆) δ=7.43-7.28 (m, 2H), 7.23 (br d, J=8.4 Hz, 2H), 7.08-7.04 (m, 1H), 6.55-6.42 (m, 2H), 4.53-4.14 (m, 4H), 4.11-3.88 (m, 6H), 3.72 (br d, J=7.6 Hz, 4H), 3.61-3.44 (m, 1H), 3.18 (br s, 2H), 3.09-2.91 (m, 2H), 2.80-2.51 (m, 5H), 2.49-2.42 (m, 3H), 2.40-2.25 (m, 3H), 2.13 (br d, J=1.2 Hz, 2H), 1.91 (br d, J=10.0 Hz, 1H), 1.85-1.54 (m, 5H), 1.47-1.24 (m, 13H), 1.20-1.16 (m, 3H), 1.13-0.94 (m, 2H).

### Step 2: Synthesis of I-104-3

To a solution of I-104-1.2 (0.1 g, 128.54 μmol, 1 eq) in THF (0.3 mL), H₂O (0.3 mL), MeOH (0.3 mL) was added LiOH·H2O (16.18 mg, 385.61 μmol, 3 eq). The mixture was stirred at 25° C. for 1 hr. The mixture was removed the ether on a rotary evaporator to give product. The crude was used into the next step without further purification. I-104-3 (150 mg, crude) as yellow solid. LCMS (Method E): R^{t}=0.491 min, [M+H]⁺=750.5. ¹H NMR (400 MHZ, DMSO-d₆) δ=7.42-7.28 (m, 2H), 7.27-7.17 (m, 2H), 7.11-7.01 (m, 1H), 6.55-6.41 (m, 2H), 4.50-4.34 (m, 2H), 4.30-4.16 (m, 2H), 4.09-3.91 (m, 4H), 3.80-3.69 (m, 4H), 3.07-2.89 (m, 2H), 2.80-2.64 (m, 2H), 2.42-2.23 (m, 6H), 2.10 (br s, 2H), 1.91 (br d, J=8.8 Hz, 1H), 1.59 (s, 11H), 1.50-1.20 (m, 13H), 1.13-0.93 (m, 3H),

### Step 3: Synthesis of I-104-5

To a solution of I-104-3 (124.08 mg, 147.37 μmol, 1.1 eq, 2FA) in DMF (1 mL) was added EDCI (77.05 mg, 401.91 μmol, 3 eq), NMM (67.75 mg, 669.85 μmol, 73.64 μL, 5 eq), and HOAt (27.35 mg, 200.95 μmol, 28.11 μL, 1.5 eq). I-104-4 (0.08 g, 133.97 μmol, 1 eq) was added. The reaction mixture was stirred at 25° C. for 1.5 hr. The reaction mixture was quenched by addition H₂O 3 mL at 25° C., then extracted with EA 6 mL (2 mL*3). The combined organic layers were dried over Na₂SO₄, filtered and concentrated under reduced pressure to give a residue. The crude product was purified by reversed-phase HPLC (0.1% HCl). After purification, the eluent was concentrated to remove organic solvents and then lyophilized to give product. I-104-5 (0.08 g, 133.97 μmol, 1 eq) (140 mg, 91.64 μmol, 68.41% yield, 87% purity) as white solid. LCMS (Method E): R^{t}=0.779 min, [M+H]⁺=1328.9. ¹H NMR (400 MHZ, DMSO-d6) δ=12.87-12.47 (m, 1H), 11.76-11.46 (m, 1H), 8.46-8.27 (m, 2H), 7.52-7.30 (m, 7H), 7.30-7.16 (m, 3H), 7.11-7.02 (m, 1H), 6.96 (br d, J=2.4 Hz, 1H), 6.58-6.43 (m, 2H), 5.04-4.84 (m, 1H), 4.50-4.34 (m, 3H), 4.32-4.18 (m, 4H), 4.12-3.92 (m, 6H), 3.86-3.68 (m, 8H), 3.40-3.18 (m, 9H), 3.15-2.91 (m, 9H), 2.82-2.57 (m, 4H), 2.36-2.06 (m, 7H), 1.99-1.88 (m, 2H), 1.83-1.67 (m, 3H), 1.51-1.29 (m, 20H), 1.29-1.13 (m, 3H).

### Step 4: Synthesis of I-104

A suspension of I-104-5 (140 mg, 105.34 μmol, 1 eq) in HCl (0.1 M, 4.21 mL, 4 eq) was stirred at 100° C. for 0.5 h. The mixture was added NaHCO₃ to adjust pH=9˜10, and then lyophilized to give residue. The crude was purified by Prep-HPLC (column: Waters Xbridge 150*25 mm*5 um; mobile phase: [water (NH₃·H2O 0.1%)-ACN]; gradient: 30%-60% B over 15 min). After prep-HPLC purification, the eluent was concentrated to remove organic solvents and then lyophilized to give product. I-104 (41.98 mg, 36.67 μmol, 34.81% yield, 98.6% purity) as white solid. LCMS (Method G): R^{t}=0.653 min, [M+H]⁺=1018.7. SFC: R^{t}=3.218 min, 5.979 min. ¹H NMR (400 MHZ, DMSO-d₆) δ=11.66 (br s, 1H), 8.75 (br d, J=8.0 Hz, 1H), 8.11 (s, 1H), 7.41-7.29 (m, 6H), 7.26-7.12 (m, 4H), 6.58-6.56 (m, 1H), 6.52-6.40 (m, 2H), 4.92-4.76 (m, 1H), 4.55-4.28 (m, 4H), 4.09-3.90 (m, 2H), 3.83-3.69 (m, 4H), 3.62-3.41 (m, 9H), 3.41-3.37 (m, 1H), 3.32-3.25 (m, 2H), 3.08 (s, 2H), 3.05-2.93 (m, 2H), 2.76-2.63 (m, 2H), 2.61-2.55 (m, 1H), 2.42-2.07 (m, 18H), 1.99-1.83 (m, 5H), 1.79-1.67 (m, 4H), 1.65-1.54 (m, 1H), 1.46-1.38 (m, 3H), 1.32-1.28 (m, 3H), 1.09-0.95 (m, 2H).

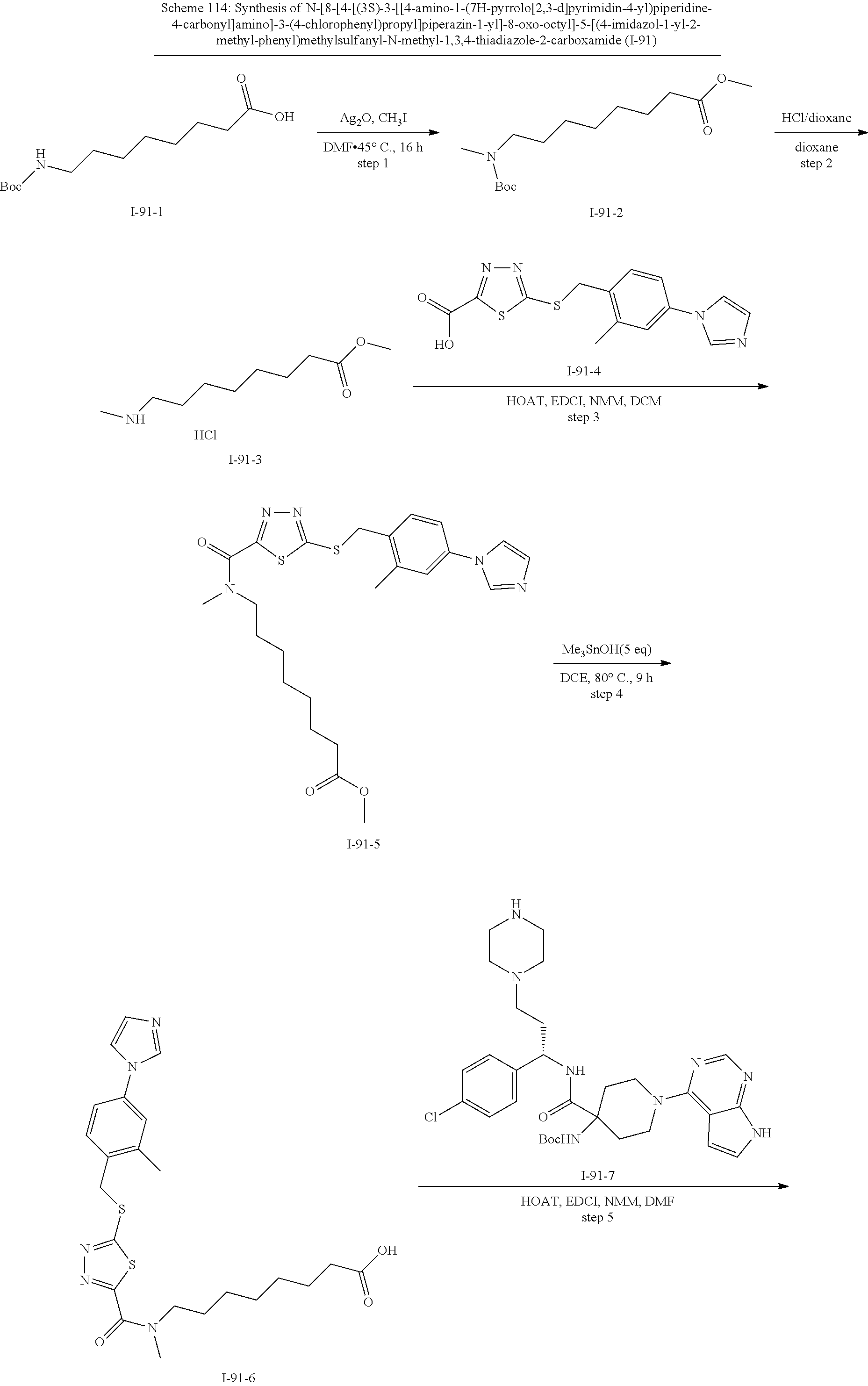

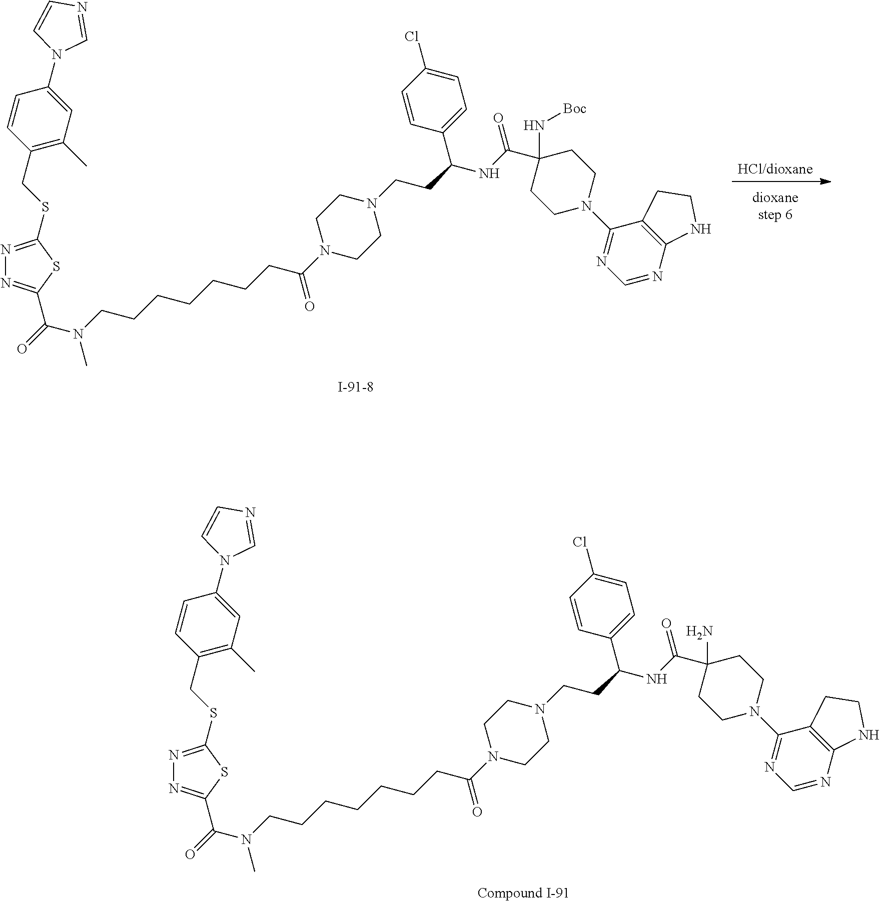 [SMILES: Cc1cc(-n2ccnc2)ccc1CSc1nnc(C(=O)N(C)CCCCCCCC(=O)N2CCN(CC[C@H](NC(=O)C3(NC(=O)OC(C)(C)C)CCN(c4ncnc5c4CCN5)CC3)c3ccc(Cl)cc3)CC2)s1 ; "HCl/dioxane" ; Cc1cc(-n2ccnc2)ccc1CSc1nnc(C(=O)N(C)CCCCCCCC(=O)N2CCN(CC[C@H](NC(=O)C3(N)CCN(c4ncnc5c4CCN5)CC3)c3ccc(Cl)cc3)CC2)s1]

### Step 1: Synthesis of I-91-2

To a solution of I-91-1 (2 g, 7.71 mmol, 1 eq) in DMF (30 mL) was added Ag₂O (8.94 g, 38.56 mmol, 5 eq) and CH₃I (6.57 g, 46.27 mmol, 2.88 mL, 6 eq). The resulting suspension was stirred at 45° C. for 16 h under dark and N₂. The reaction mixture was cooled to 20° C., and filtered, the cake was washed with EtOAc (60 mL). The combined filtrate was washed with brine (60 mL*4), dried over Na₂SO₄, filtered, and concentrated under vacuum to give a residue. The residue was purified by flash silica gel chromatography (ISCO®; 220 g SepaFlash® Silica Flash Column, Eluent of 10˜16% Ethyl acetate/Petroleum ether gradient @ 100 mL/min), the eluent was concentrated under vacuum. I-91-2 (1.32 g. 4.59 mmol, 59.56% yield) was obtained as colorless oil. ¹H NMR (400 MHZ, CHLOROFORM-d) δ=3.67 (s, 3H), 3.19 (t, J=7.3 Hz, 2H), 2.83 (s, 3H), 2.31 (t, J=7.5 Hz, 2H), 1.68-1.61 (m, 2H), 1.56-1.41 (m, 11H), 1.38-1.22 (m, 6H).

### Step 2: Synthesis of I-91-3

To a solution of I-91-2 (1.3 g, 4.52 mmol, 1 eq) in dioxane (5 mL) was added HCl/dioxane (4M, 5 mL) dropwise at 20° C., and stirred for 3 h. The reaction mixture was concentrated under reduced pressure to give I-91-3 (0.98 g, crude, HCl) as a white solid. ¹H NMR (400 MHZ, DMSO-d₆) δ=8.85 (br s, 2H), 3.59 (s, 3H), 2.82 (br t, J=6.8 Hz, 2H), 2.50 (br s, 3H), 2.30 (t, J=7.4 Hz, 2H), 1.66-1.46 (m, 4H), 1.27 (br s, 6H).

### Step 3: Synthesis of I-91-5

To a solution of I-91-4 (0.9 g, 2.53 mmol, 1 eq, Na) in DCM (20 mL) was added HOAt (517.04 mg, 3.80 mmol, 1.5 eq), NMM (1.54 g, 15.19 mmol, 1.67 mL, 6 eq) and EDCI (1.46 g. 7.60 mmol, 3 eq) at 20° C., then I-91-3 (623.26 mg, 2.79 mmol, 1.1 eq, HCl) was added. The reaction mixture was stirred at 20° C. for 16 hrs under N₂. The reaction solution (together with, 0.1 g of I-91-4) was diluted with water (20 mL), DCM phase was separated, and the resulting water phase was extracted with DCM (20 mL). The combined organic phase was washed with brine (10 mL), dried over Na₂SO₄, filtered and concentrated under vacuum to give a residue. The residue, dissolved in ACN (5 mL), purified by reversed phase preparative HPLC (0.1% FA conditions) (105 g of XB—C18, 20-35 μm, 100 Å) Mobile phase: A for H₂O (0.1% FA v/v) and B for acetonitrile; Gradient: B 10%-70% in 25 min: Flow rate: 60 ml/min; Column temperature: R.T. Wavelength: 220 nm/254 nm). After purification, the eluent was concentrated to remove organic solvents, then it was neutralized with NaHCO₃ to pH=8-9, extracted with DCM (20 mL*2), the combined organic phase was dried over Na₂SO₄, filtered and concentrated to give I-91-5 (1.3 g, 2.54 mmol, 90.18% yield, 97.916% purity) as yellow oil. LCMS (Method E): R^{t}=0.480 min, [M+H]⁺=502.3. ¹H NMR (400 MHZ, DMSO-d₆) δ=8.25 (s, 1H), 7.74 (s, 1H), 7.57 (s, 1H), 7.55 (d, J=3.1 Hz, 1H), 7.50-7.44 (m, 1H), 7.09 (s, 1H), 4.69 (d, J=1.9 Hz, 2H), 3.80 (br t, J=7.4 Hz, 1H), 3.57 (d, J=2.3 Hz, 3H), 3.45 (t, J=7.3 Hz, 1H), 3.36 (s, 1H), 3.02 (s, 2H), 2.46 (s, 3H), 2.28 (dt, J=4.4, 7.3 Hz, 2H), 1.66-1.43 (m, 4H), 1.30-1.17 (m, 6H).

### Step 4: Synthesis of I-91-6

To a solution of I-91-5 (0.18 g, 358.81 μmol, 1 eq) in DCE (3 mL) was added hydroxy (trimethyl) stannane (324.40 mg, 1.79 mmol, 5 eq). The mixture was stirred at 80° C. for 9 hr. The reaction mixture was filtered, the filtrated was concentrated under reduced pressure to give a residue. The residue was purified by flash silica gel chromatography (ISCO®; 20 g SepaFlash® Silica Flash Column, Eluent of 5˜15% MeOH/DCM gradient (a, 60 mL/min), the eluent was concentrated under vacuum. I-91-6 (0.160 g, 270.43 μmol, 75.37% yield, 82.421% purity) as yellow oil. LCMS (Method E): R^{t}=0.442 min, [M+H]⁺=488.2.

### Step 5: Synthesis of I-91-8

To a solution of I-91-7 (0.107 g, 179.18 μmol, 1 eq) and I-91-6 (116.61 mg, 197.10 μmol, 1.1 eq) in DMF (3 mL) was added HOAt (36.58 mg, 268.78 μmol, 1.5 eq), EDCI (103.05 mg, 537.55 μmol, 3 eq) and NMM (108.74 mg, 1.08 mmol, 118.20 μL, 6 eq), then stirred at 15° C. for 16 hr under N₂. The reaction solution was diluted with EtOAc (10 mL), washed with brine (5 mL*4), dried over Na₂SO₄, filtered and concentrated under vacuum to give a residue. The residue, dissolved in ACN (5 mL), purified by reversed phase preparative HPLC (0.1% FA conditions) (105 g of XB—C18, 20-35 μm, 100 Å) Mobile phase: A for H₂O (0.1% FA v/v) and B for acetonitrile; Gradient: B 5%-65% in 25 min: Flow rate: 60 ml/min; Column temperature: R.T. Wavelength: 220 nm/254 nm). After purification, the eluent was concentrated to remove organic solvents, then it was neutralized with NaHCO₃ to pH=8-9, extracted with DCM (5 mL*2), the combined organic phase was dried over Na₂SO₄, filtered and concentrated to give I-91-8 (0.11 g. 103.11 μmol, 57.55% yield, 100% purity) as a yellow solid. LCMS (Method E): R^{t}=0.449 min, [M+H]⁺=1066.9.

### Step 6: Synthesis of I-91

To a solution of I-91-8 (0.11 g, 103.11 μmol, 1 eq) in dioxane (2 mL) was added HCl/dioxane (4M, 2 mL) at 15° C., then stirred at 15° C. for 0.5 h. The reaction suspension was concentrated under reduced pressure to give a residue. The residue, dissolved in ACN (5 mL), purified by reversed phase preparative HPLC (0.1% FA conditions) (105 g of XB—C18, 20-35 μm, 100 Å) Mobile phase: A for H₂O (0.1% FA v/v) and B for acetonitrile; Gradient: B 0%-65% in 20 min: Flow rate: 60 ml/min; Column temperature: R.T. Wavelength: 220 nm/254 nm). After purification, the eluent was concentrated to remove organic solvents, then it was lyophilized to give I-91 (29.55 mg, 29.18 μmol, 28.30% yield, 100% purity, FA) as an orange solid, LCMS (Method E): R^{t}=0.432 min, [M+H]⁺=966.8. SFC: R^{t}=1.324 min, ¹H NMR (400 MHZ, DMSO-d₆) δ=11.70 (br s, 1H), 8.81 (br d, J=7.9 Hz, 1H), 8.25 (s, 1H), 8.16-8.12 (m, 2H), 7.73 (d, J=1.0 Hz, 1H), 7.60-7.52 (m, 2H), 7.46 (br d, J=8.3 Hz, 1H), 7.40-7.28 (m, 4H), 7.21-7.15 (m, 1H), 7.09 (s. 1H), 6.62 (br d, J=0.9 Hz. 1H), 4.86 (br d, J=6.0 Hz. 1H), 4.69 (s, 2H), 4.47 (br d, J=12.8 Hz, 2H). 3.87-3.75 (m, 1H), 3.61-3.48 (m, 3H), 3.48-3.40 (m, 6H), 3.36 (s, 2H), 3.02 (s, 2H), 2.46 (d, J=1.9 Hz. 3H), 2.35-2.18 (m, 8H), 2.15-2.03 (m, 2H), 1.93-1.80 (m, 2H), 1.70-1.52 (m, 4H), 1.45 (br d, J=6.8 Hz. 2H), 1.32-1.18 (m, 6H).

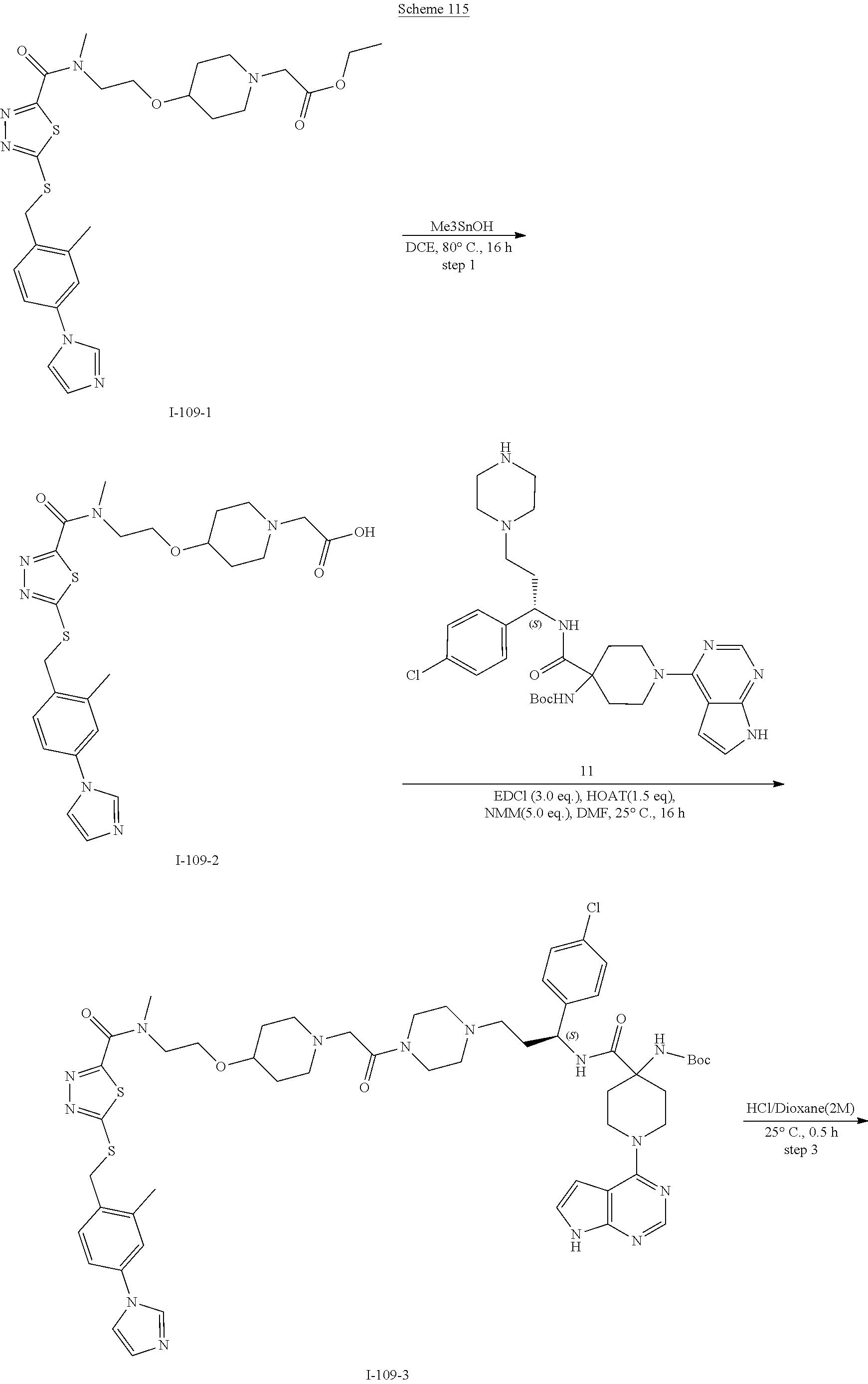

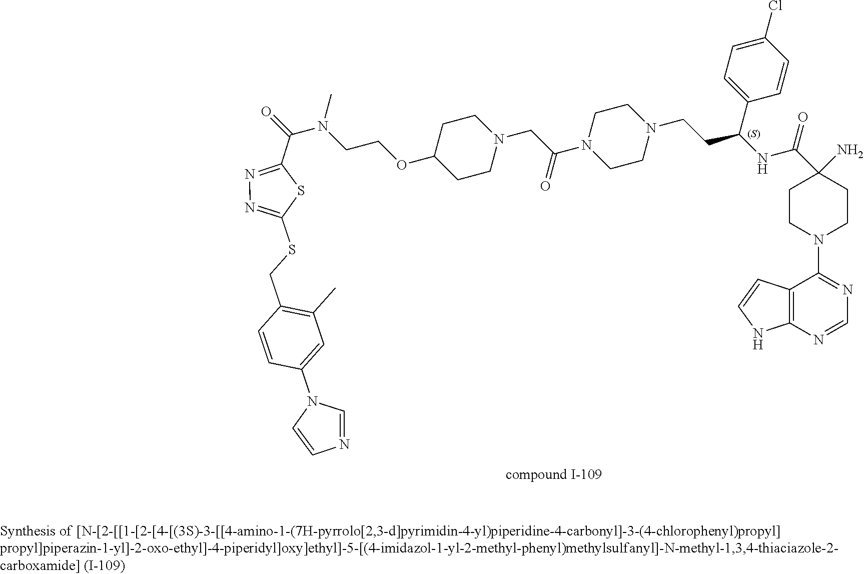

### Step 1: Synthesis of I-109-2

To a solution of I-109-1 (0.5 g, 894.91 μmol, 1 eq) in DCE (5 mL) was added hydroxy (trimethyl) stannane (809.09 mg, 4.47 mmol, 5 eq). The mixture was stirred at 80° C. for 12 h. The reaction mixture was filtered and the filtrate was concentrated to give I-109-2 (500 mg, crude) as a yellow oil. LCMS (Method D): R^{t}=0.266 min, (M+H)=531.1.

### Step 2: Synthesis of I-109-4

The I-109-2 (450 mg, 848.00 μmol, 1 eq) in DMF (4.5 mL) was added EDCI (487.69 mg, 2.54 mmol, 3 eq), NMM (428.86 mg, 4.24 mmol, 466.15 μL, 5 eq), I-109-3 (506.38 mg, 848.00 μmol, 1 eq) and HOAt (173.13 mg, 1.27 mmol, 177.94 μL, 1.5 eq). The mixture was stirred at 25° C. for 1 h. The reaction mixture was washed with H₂O (20 mL) at 25° C., and mixture was extracted with EA (20 ml*3). The combined organic phase was dried with anhydrous sodium sulfate, filtered and the filtrate was concentrated to give crude product. The crude product was purified by prep-HPLC (0.1% NH3·H2O condition) and the eluent was lyophilized to give I-109-4 (500 mg, 441.52 μmol, 52.07% yield, 98% purity) as a yellow solid. SFC: R^{t}=0.995 min. LCMS (Method D): R^{t}=0.652 min, (M+H)=1109.6.

### Step 3: Synthesis of I-109

A mixture of I-109-4 (300 mg, 270.32 μmol, 1 eq) in HCl/dioxane (2 M, 3.00 mL, 22.20 eq) was stirred at 25° C. for 0.5 h. Concentrated under reduced pressure to give a residue. Then H₂O (30 ml) was added into the product, the mixture was lyophilized to give I-109 (211.85 mg, 188.11 μmol, 69.59% yield, 92.889% purity, HCl) as a yellow solid. SFC: R^{t}=0.736 min. LCMS: R^{t}=0.621 min, (M+H)=1009.5. 1H NMR (400 MHZ, METHANOL-d4) δ=9.48 (s, 1H), 8.39 (s, 1H), 8.09 (s, 1H), 7.78 (s, 1H), 7.72 (d, J=8.4 Hz, 1H), 7.66 (s, 1H), 7.55 (d, J=8.0 Hz, 1H), 7.48-7.39 (m, 3H), 7.35 (d, J=8.0 Hz, 2H), 7.02 (d, J=3.6 Hz, 1H), 5.07-5.04 (m, 1H), 4.75 (s, 2H), 4.62 (d, J=7.6 Hz, 3H), 4.53-4.42 (m, 1H), 4.36-4.24 (m, 1H), 4.21-4.13 (m, 1H), 3.97 (s, 3H), 3.79 (s, 3H), 3.73-3.57 (m, 5H), 3.55 (d, J=3.2 Hz, 1H), 3.51-3.33 (m, 4H), 3.28-3.11 (m, 6H), 2.90-2.79 (m, 1H), 2.79-2.69 (m, 1H), 2.62-2.45 (m, 4H), 2.40-2.17 (m, 3H), 2.11-2.00 (m, 3H), 1.93-1.67 (m, 1H).

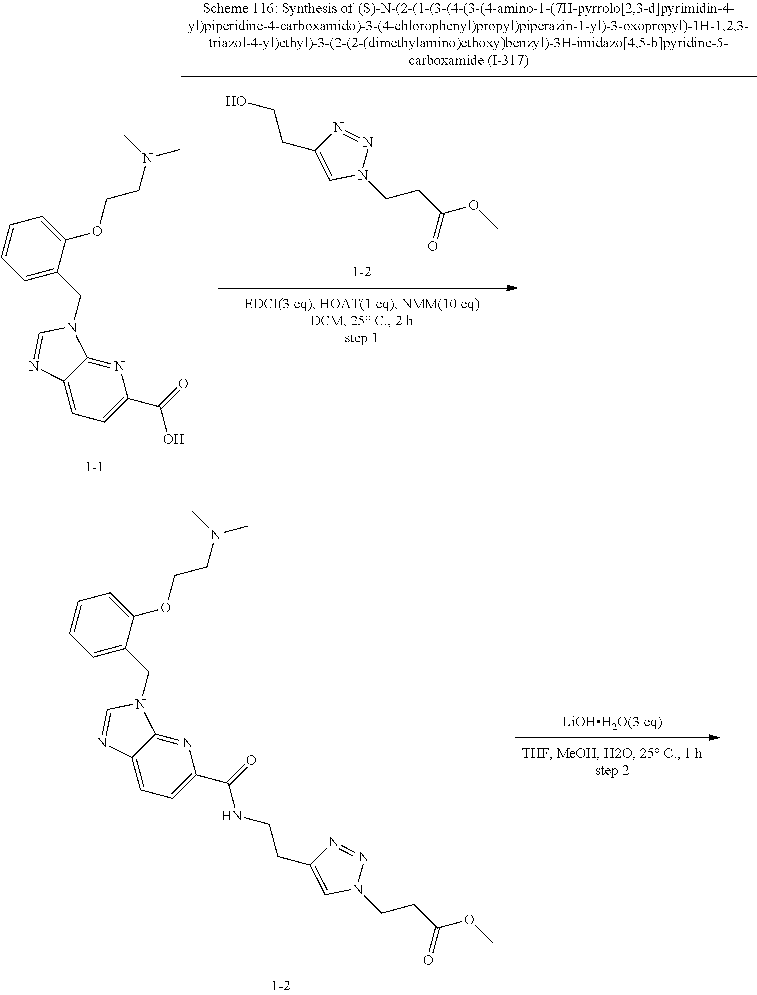 [SMILES: CN(C)CCOc1ccccc1Cn1cnc2ccc(C(=O)O)nc21 ; COC(=O)CCn1cc(CCO)nn1 ; COC(=O)CCn1cc(CCNC(=O)c2ccc3ncn(Cc4ccccc4OCCN(C)C)c3n2)nn1]

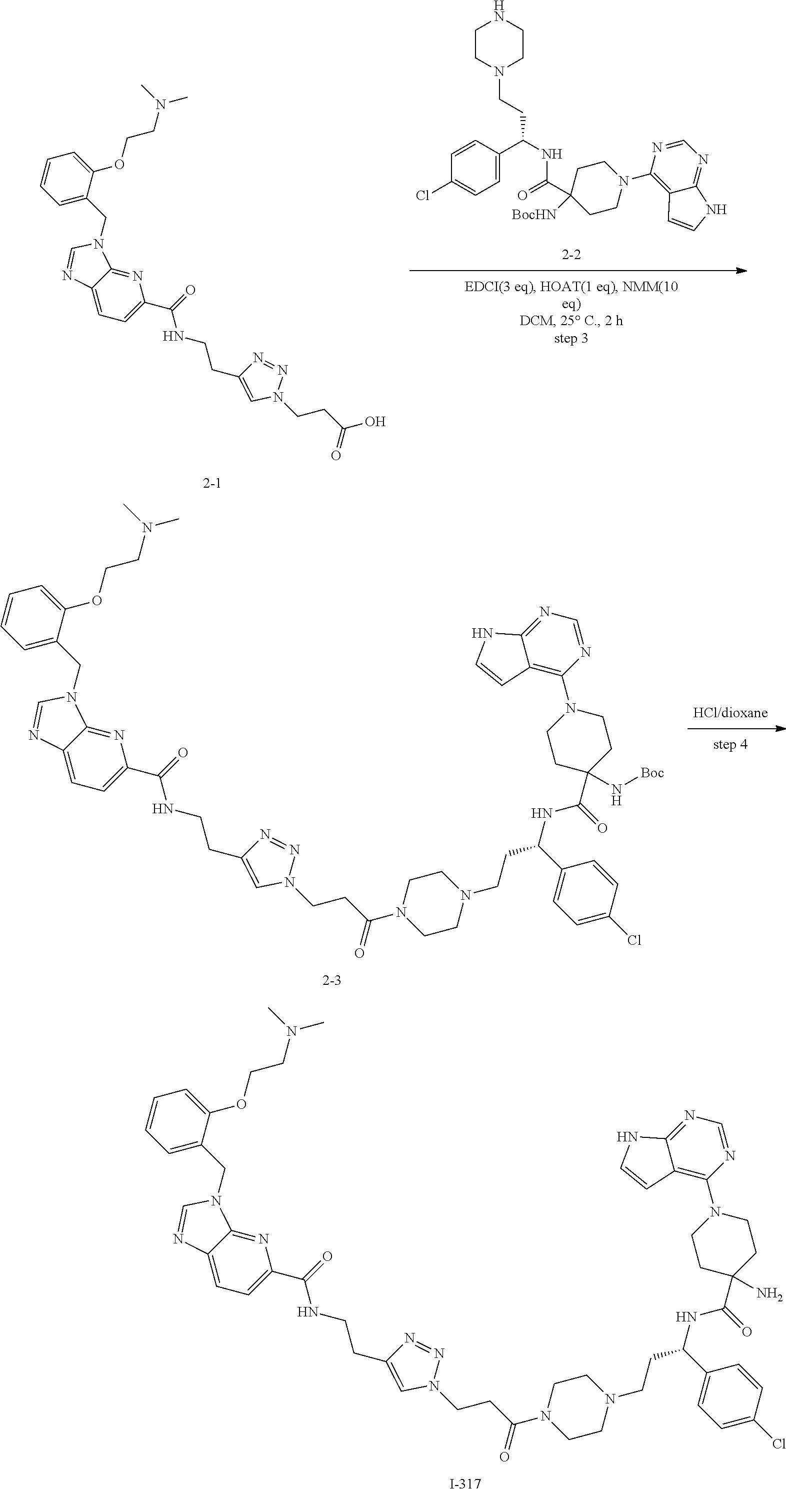 [SMILES: CN(C)CCOc1ccccc1Cn1cnc2ccc(C(=O)NCCc3cn(CCC(=O)O)nn3)nc21 ; CC(C)(C)OC(=O)NC1(C(=O)N[C@@H](CCN2CCNCC2)c2ccc(Cl)cc2)CCN(c2ncnc3[nH]ccc23)CC1 ; CN(C)CCOc1ccccc1Cn1cnc2ccc(C(=O)NCCc3cn(CCC(=O)N4CCN(CC[C@H](NC(=O)C5(NC(=O)OC(C)(C)C)CCN(c6ncnc7[nH]ccc67)CC5)c5ccc(Cl)cc5)CC4)nn3)nc21 ; CN(C)CCOc1ccccc1Cn1cnc2ccc(C(=O)NCCc3cn(CCC(=O)N4CCN(CC[C@H](NC(=O)C5(N)CCN(c6ncnc7[nH]ccc67)CC5)c5ccc(Cl)cc5)CC4)nn3)nc21]

### Step 1: Synthesis of 1-2

To a solution of 1-1 (100 mg, 293.79 μmol, 1 eq) and 1-2 (82.74 mg, 352.55 μmol, 1.2 eq, HCl) in DCM (1 mL) was added EDCI (168.96 mg, 881.38 μmol, 3 eq), HOAt (39.99 mg, 293.79 μmol, 41.10 μL, 1 eq) and NMM (297.16 mg, 2.94 mmol, 323.00 μL, 10 eq). The mixture was stirred at 25° C. for 2 h. The reaction mixture was quenched by addition H₂O 1 mL, and then diluted with DCM 1 mL and extracted with DCM 6 mL (2 mL*3). The combined organic layers were dried over anhydrous Na₂SO₄, filtered and concentrated under reduced pressure to give a residue. The residue was purified by prep-HPLC (column: Phenomenex luna C18 150*25 mm*10 um; mobile phase: [water (FA)-ACN]; gradient: 8%-38% B over 9 min), then concentrated to remove organic solvents and lyophilized to give 1-2 (80 mg, 141.19 μmol, 48.06% yield, 100% purity, FA) as a yellow oil. LCMS (Method E): R^{t}=0.387 min, (M+H)=521.3. ¹H NMR (400 MHz, METHANOL-d4) δ=8.51 (s, 1H), 8.45 (s, 1H), 8.20-8.16 (m, 1H), 8.13-8.07 (m, 1H), 7.84 (s, 1H), 7.43 (d, J=7.6 Hz, 1H), 7.39-7.34 (m, 1H), 7.07 (d, J=8.4 Hz, 1H), 7.02-6.98 (m, 1H), 5.65 (s, 2H), 4.63-4.60 (m, 2H), 4.28-4.26 (m, 2H), 3.79-3.76 (m, 2H), 3.60 (s, 3H), 3.15-3.12 (m, 2H), 3.09-3.05 (m, 2H), 2.96-2.92 (m, 2H), 2.62 (s, 6H).

### Step 2: Synthesis of 2-1

To a solution of 1-2 (200 mg, 384.19 μmol, 1 eq) in THF (2 mL), MeOH (2 mL) and H₂O (1 mL) was added LiOH·H₂O (48.36 mg, 1.15 mmol, 3 eq) and. The mixture was stirred at 25° C. for 1 h. The reaction mixture was concentrated under reduced pressure to give a residue, and purified by reversed phase (NH3·H2O), then concentrated to remove organic solvents and lyophilized to give 2-1 (180 mg, 344.68 μmol, 89.72% yield, 97% purity) as a white solid. LCMS (Method E): R^{t}=0.361 min, (M+H)=507.3.

### Step 3: Synthesis of 2-3

To a solution of 2-1 (90 mg, 177.67 μmol, 1 eq) and 2-1 (116.71 mg, 195.44 μmol, 1.1 eq) in DCM (2 mL) was added EDCI (102.18 mg, 533.01 μmol, 3 eq), HOAt (24.18 mg, 177.67 μmol, 24.85 μL, 1 eq) and NMM (179.71 mg, 1.78 mmol, 195.34 μL, 10 eq). The mixture was stirred at 25° C. for 2 h. The reaction mixture was quenched by addition H₂O 2 mL, and then diluted with DCM 2 mL and extracted with DCM 6 mL (2 mL*3). The combined organic layers were dried over anhydrous Na₂SO₄, filtered and concentrated under reduced pressure to give 2-3 (200 mg, crude) as a yellow solid and used into next step without further purification. LCMS (Method E): R^{t}=0.408 min, (M+H)=1085.8.

### Step 4: Synthesis of I-317

To a solution of 2-3 (180 mg, 165.79 μmol, 1 eq) in DCM (4 mL) was added HCl/dioxane (4 M, 2 mL, 48.25 eq). The mixture was stirred at 25° C. for 0.5 h. The reaction mixture was concentrated under reduced pressure to give a residue. The residue was purified by prep-HPLC (column: Waters Xbridge 150*25 mm*5 um; mobile phase: [water (NH4HCO3)-ACN]; gradient: 22%-52% B over 9 min), then concentrated to remove organic solvents and lyophilized to give I-317 (76.06 mg, 75.18 μmol, 45.35% yield, 97.42% purity) as a yellow oil. LCMS (Method E): R^{t}=0.377 min, (M+H)=985.6. ¹H NMR (400 MHz, METHANOL-d4) δ=8.60 (s, 1H), 8.15-8.05 (m, 3H), 7.84 (s, 1H), 7.49-7.47 (m, 1H), 7.34-7.27 (m, 5H), 7.09 (d, J=3.6 Hz, 1H), 7.01-6.91 (m, 2H), 6.58 (d, J=3.6 Hz, 1H), 5.60 (s, 2H), 4.97-4.93 (m, 1H), 4.65-4.62 (m, 2H), 4.53-4.43 (m, 2H), 4.09-4.06 (m, 2H), 3.80-3.76 (m, 2H), 3.66-3.50 (m, 4H), 3.40 (d, J=3.2 Hz, 2H), 3.09-3.06 (m, 2H), 2.97-2.94 (m, 2H), 2.64-2.61 (m, 2H), 2.34-2.29 (m, 2H), 2.29-2.21 (m, 10H), 2.19-2.08 (m, 2H), 2.04-1.87 (m, 2H), 1.62-1.50 (m, 2H). SFC: R^{t}=0.911 min, ee value=100%

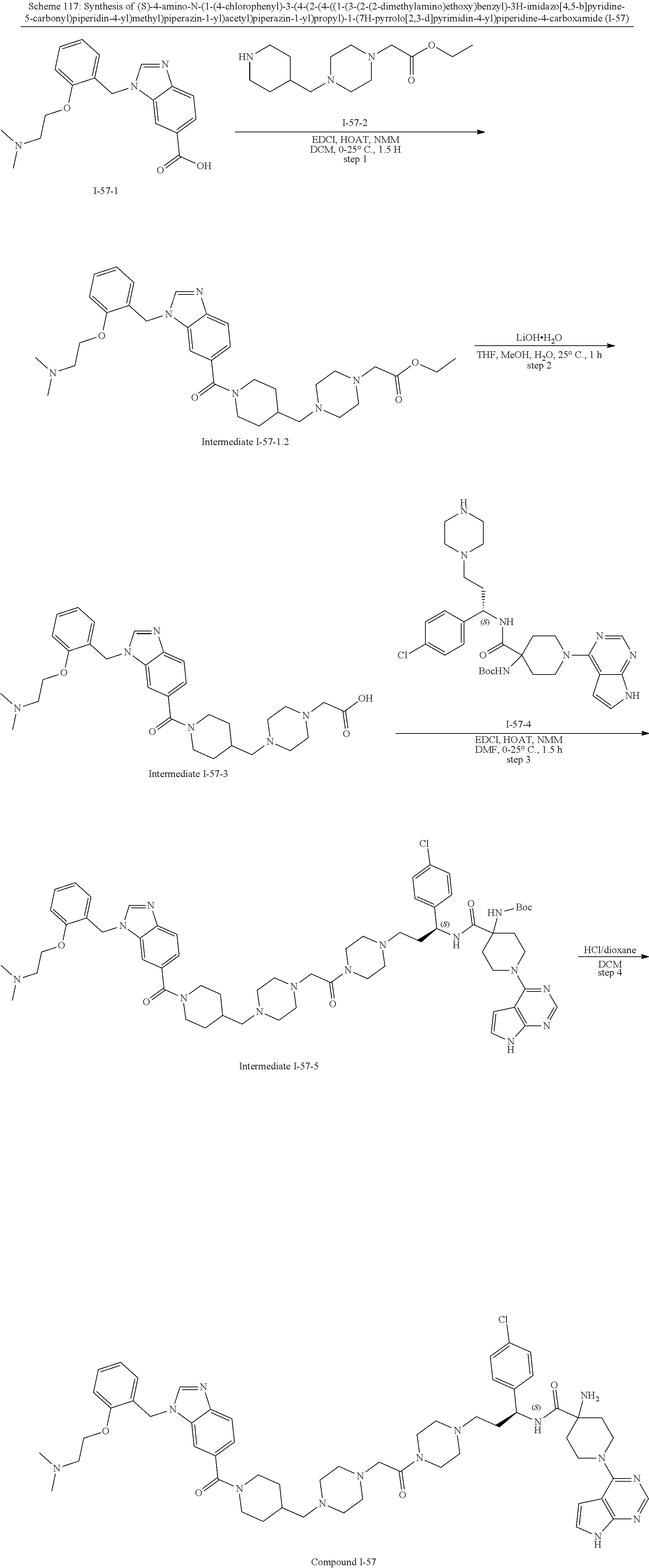

### Step 1: Synthesis of I-57-1.2

A mixture of I-57-1 (0.1 g, 293.79 μmol, 1 eq), I-57-2 (107.83 mg, 352.55 μmol, 1.2 eq, HCl) and HOAt (39.99 mg, 293.79 μmol, 41.10 μL, 1 eq) in DCM (1 mL) was added EDCI (112.64 mg, 587.59 μmol, 2 eq) and NMM (148.59 mg, 1.47 mmol, 161.51 μL, 5 eq). The mixture was stirred at 25° C. for 12 hrs. The mixture was concentrated under vacuum. The crude product was purified by reverse-phase HPLC (0.1% FA condition) and concentrated under vacuum to remove MeCN and dried by lyophilization to get I-57-1.2 (110 mg, 185.89 μmol, 63.27% yield) as yellow oil. LCMS (Method G): R^{t}=0.554 min, (M+H)+=592.3. ¹H NMR (400 MHZ, DMSO-d₆) δ=8.68 (s, 1H), 8.25-8.10 (m, 3H), 7.42 (d, J=8.3 Hz, 1H), 7.35-6.82 (m, 4H), 5.43 (s, 2H), 4.56-4.41 (m, 1H), 4.15-4.01 (m, 5H), 3.01-2.88 (m, 3H), 2.82-2.72 (m, 2H), 2.71-2.63 (m, 2H), 2.40-2.30 (m, 4H), 2.26 (s, 7H), 2.17-2.10 (m, 3H), 1.86-1.68 (m, 3H), 1.57-1.42 (m, 1H), 1.18 (t, J=7.1 Hz, 3H). ¹H NMR (400 MHZ, DMSO-d₆) δ=8.68 (s, 1H), 8.14 (d, J=8.4 Hz, 1H), 7.42 (d, J=8.0 Hz, 1H), 7.31-7.21 (m, 1H), 7.11 (br d, J=7.2 Hz, 1H), 7.04 (d, J=8.4 Hz, 1H), 6.88-6.86 (m, 1H), 5.43 (s, 2H), 4.48 (br d, J=13.6 Hz, 1H), 4.13-4.03 (m, 4H), 3.73-3.62 (m, 1H), 3.32 (br s, 6H), 2.98-2.87 (m, 1H), 2.85-2.73 (m, 1H), 2.65-2.62 (m, 2H), 2.41-2.28 (m, 4H), 2.23 (s, 6H), 2.14 (br d, J=4.0 Hz, 2H), 1.87-1.71 (m, 2H), 1.57-1.44 (m, 1H), 1.19-1.16 (m, 3H), 1.13-0.99 (m, 2H).

### Step 2: Synthesis of I-57-3

To a solution of I-57-1.2 (110 mg, 185.89 μmol, 1 eq) in THF (0.4 mL), MeOH (0.2 mL) and H₂O (0.2 mL) was added NaOH (22.31 mg, 557.68 μmol, 3 eq). The mixture was stirred at 25° C. for 2 hr. The mixture was adjust pH=4 with 2M HCl and concentrated under vacuum. The crude product was purified by reverse-phase HPLC (0.1% FA condition) and concentrated under vacuum to remove MeCN and dried by lyophilization to get I-57-3 (0.1 g. 177.40 μmol, 95.43% yield) as yellow solid. LCMS (Method G): R^{t}=0.317 min, (M+H)+=564.2.

### Step 3: Synthesis of I-57-5

A mixture of I-57-3 (0.1 g, 177.40 μmol, 1 eq), I-57-4 (105.94 mg, 177.40 μmol, 1 eq) and HOAt (24.15 mg, 177.40 μmol, 24.82 μL, 1 eq) in DCM (1 mL) was added EDCI (68.02 mg, 354.81 μmol, 2 eq) and NMM (89.72 mg, 887.01 μmol, 97.52 μL, 5 eq). The mixture was stirred at 25° C. for 1 hrs. The mixture was concentrated under vacuum. The residue was purified by Pre-HPLC (column: Waters Xbridge 150*25 mm*5 um; mobile phase: [water (NH4HCO3)-ACN]; gradient: 28%-58% B over 9 min) and dried by lyophilization to get I-57-5 (72 mg, 63.00 μmol, 35.51% yield) as white solid. LCMS (Method G): R^{t}=0.649 min, (M/2+H)+=572.2.

### Step 4: Synthesis of I-57

A mixture of I-57-5 (72 mg, 63.00 μmol, 1 eq) in DCM (2 mL) was added HCl/dioxane (4 M, 1 mL, 63.49 eq). The mixture was stirred at 25° C. for 1.5 hr. The mixture was concentrated under vacuum to get I-57 (61.88 mg, 57.12 μmol, 90.67% yield, 99.621% purity, HCl) as white solid was obtained. LCMS (Method E): R^{t}=0.344 min, (M+H)+=1042.6. SFC: R^{t}=1.055 min. F2. ¹H NMR (400 MHZ, DMSO-d6) δ=12.90 (br s, 1H), 11.83-11.69 (m, 1H), 11.15-10.94 (m, 1H), 9.85-9.67 (m, 1H), 9.17 (br s, 1H), 9.01 (s, 1H), 8.39 (s, 1H), 8.25 (d, J=8.4 Hz, 1H), 7.54 (d, J=8.4 Hz, 1H), 7.50 (br s, 1H), 7.48-7.44 (m, 2H), 7.43-7.39 (m, 2H), 7.35-7.29 (m, 1H), 7.20-7.15 (m, 1H), 7.09 (d, J=8.4 Hz, 1H), 7.03 (br d, J=1.2 Hz, 1H), 6.97-6.96 (m, 1H), 5.72-5.59 (m, 2H), 4.96 (br s, 1H), 4.48 (br d, J=10.8 Hz, 1H), 4.44-4.40 (m, 2H), 4.39-4.27 (m, 3H), 4.21 (br d, J=1.6 Hz, 4H), 3.68 (br s, 3H), 3.60-3.54 (m, 5H), 3.49-3.47 (m, 3H), 3.38 (s, 1H), 3.35-2.97 (m, 11H), 2.89 (br s, 1H), 2.86 (d, J=4.8 Hz, 6H), 2.68-2.57 (m, 2H), 2.44-2.34 (m, 1H), 2.24-2.13 (m, 3H), 2.09-1.97 (m, 2H), 1.80-1.75 (m, 1H), 1.30-1.16 (m, 2H).

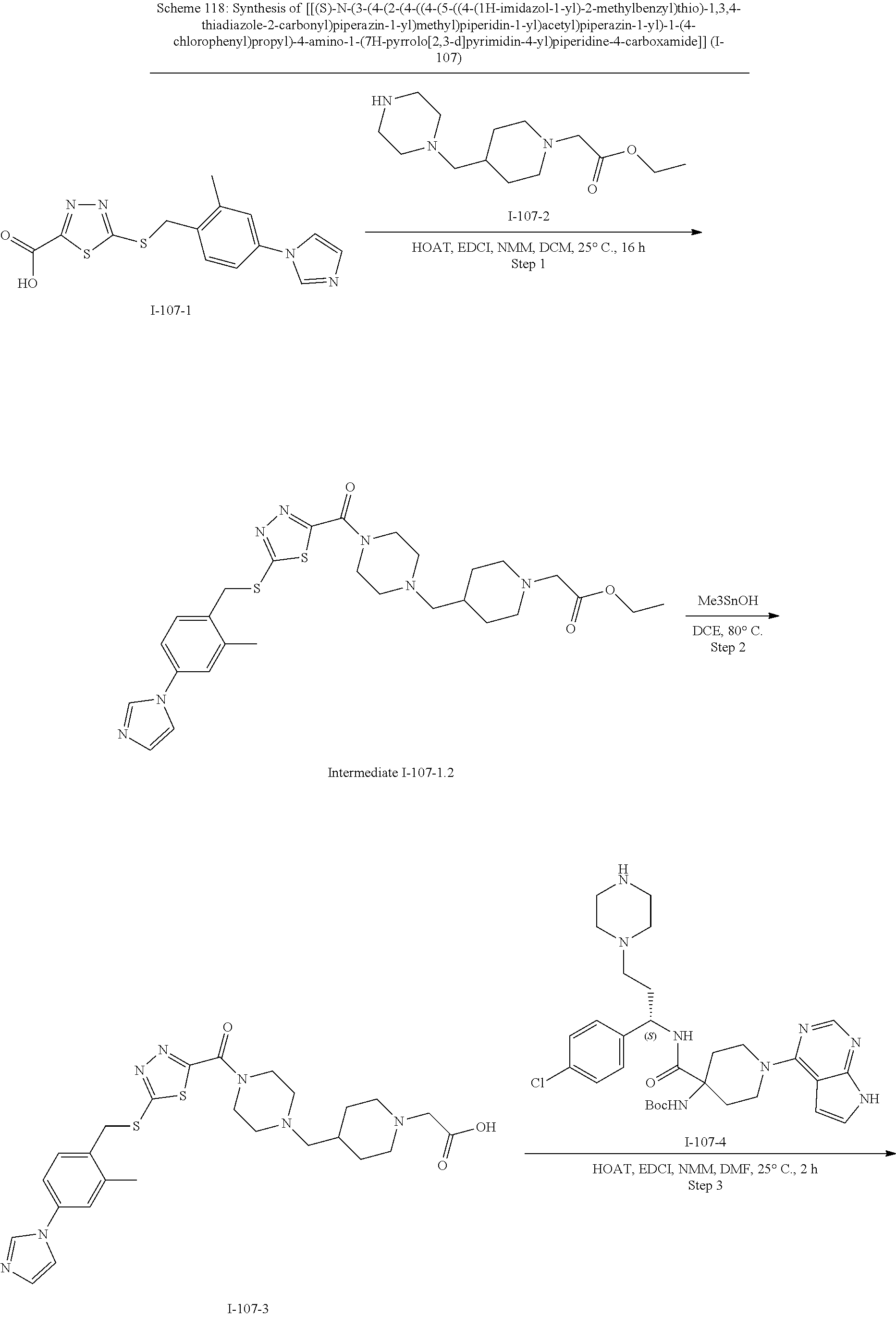

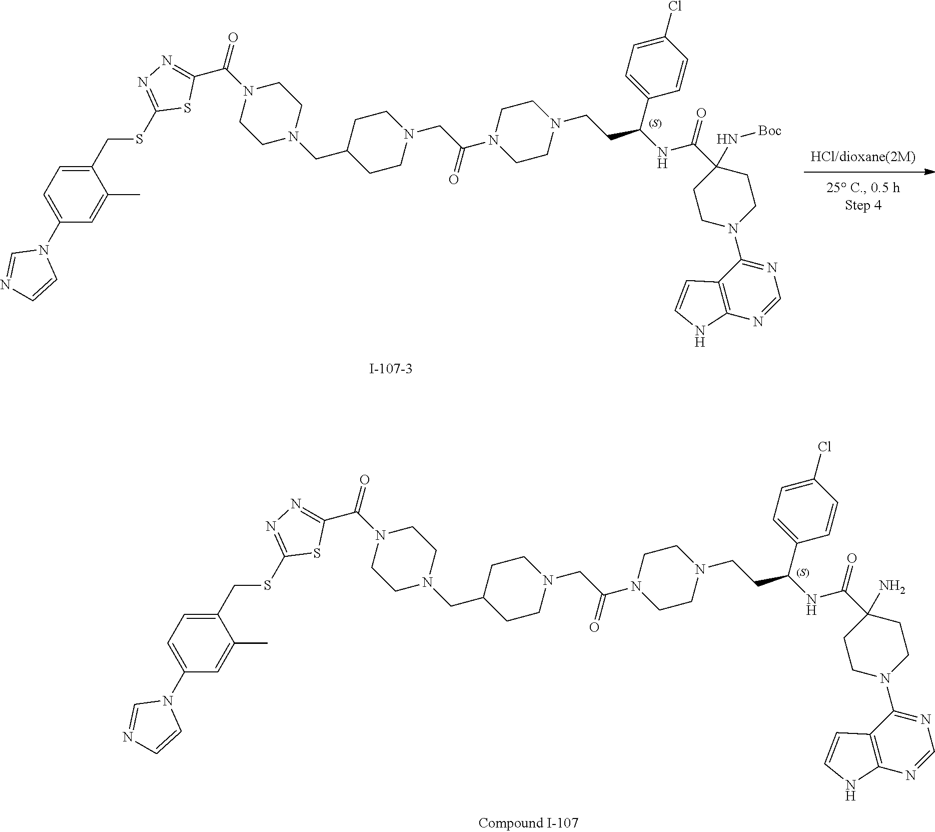

### Step 1: Synthesis of I-107-1.2

To a solution of 1-1 (1 g, 2.81 mmol, 1 eq. Na salt) and 1-2 (946.64 mg, 3.10 mmol, 1.1 eq. HCl), EDCI (1.62 g, 8.44 mmol, 3 eq) and HOAt (574.48 mg, 4.22 mmol, 590.42 μL, 1.5 eq) in DCM (10 mL) was added NMM (1.42 g, 14.07 mmol, 1.55 mL, 5 eq) and then the mixture was stirred at 25° C. for 16 hr. The reaction mixture was quenched by H₂O (20 mL), extracted with DCM (10 mL*3). The combined organic layers were dried over Na₂SO₄, filtered and concentrated under reduced pressure to give a residue. The residue was purified by reversed phase preparative HPLC (105 g) of SepaFlash® Spherical C18, 20-45 μm, 100 Å; mobile phase: A for H₂O+0.1% NH3·H₂O and B for acetonitrile; gradient: B 40-50% in 30 min, flow rate: 60 mL/min; column temperature: RT, wavelength: 220 nm/254 nm). The eluent was concentrated to remove organic solvents, then followed by lyophilization to give I-107-1.2 (1.1 g, 1.81 mmol, 64.29% yield, 96% purity) as a yellow solid. LCMS (Method B): R^{t}=0.437 min, (M+H)=584.3. ¹H NMR (400 MHZ, DMSO-d₆) δ=8.24 (s, 1H), 7.73 (s, 1H), 7.56 (d, J=8.0 Hz, 2H), 7.46-7.44 (m, 1H), 7.09 (s, 1H), 4.69 (s, 2H), 4.11-3.99 (m, 4H), 3.63 (br s, 2H), 3.18 (br s, 2H), 2.86-2.75 (m, 2H), 2.46 (s, 3H), 2.41 (br s, 4H), 2.15-2.14 (m, 4H), 1.66-1.64 (m, 2H), 1.53-1.40 (m, 1H), 1.18 (t, J=7.2 Hz, 3H), 1.15-1.04 (m, 2H).

### Step 2: Synthesis of I-107-1

To a solution of I-107-1.2 (0.2 g, 342.60 μmol, 1 eq) in DCE (2 mL) was added hydroxy (trimethyl) stannane (309.75 mg, 1.71 mmol, 5 eq). The mixture was stirred at 80° C. for 48 hr. The reaction mixture was filtered and the filter cake was washed with DCM (0.5 mL*3). The filtrate was concentrated under reduced pressure to give a residue. The residue was purified by flash silica gel chromatography (ISCO®; 12 g SepaFlash® Silica Flash Column, Eluent of 100-100% MeOH/EA) and the eluent was concentrated to give I-107-1 (0.1 g, 122.37 μmol, 35.72% yield, 68% purity) as yellow solid. LCMS (Method G): R^{t}=0.426 min, (M+H)=556.1. ¹H NMR (400 MHZ, DMSO-d₆) δ=8.24 (s, 1H), 7.73 (t, J=1.2 Hz, 1H), 7.56 (d, J=8.0 Hz, 2H), 7.46-7.44 (m, 1H), 7.09 (d, J=1.2 Hz, 1H), 4.69 (s, 2H), 4.03 (br s, 2H), 3.65-3.62 (m, 2H), 3.20-3.07 (m, 4H), 2.46 (s, 3H), 2.42 (br s, 4H), 2.28-2.23 (m, 1H), 2.16 (d, J=7.2 Hz, 2H), 1.77-1.70 (m, 2H), 1.67-1.48 (m, 2H), 1.33-1.21 (m, 2H).

### Step 3: Synthesis of I-107-3

To a solution of I-107-1 (0.05 g, 61.18 μmol, 1 eq) and I-107-2 (36.54 mg, 61.18 μmol, 1 eq), EDCI (35.19 mg, 183.55 μmol, 3 eq) and NMM (30.94 mg, 305.91 μmol, 33.63 μL, 5 eq) in DMF (0.5 mL) was added HOAt (8.33 mg, 61.18 μmol, 8.56 μL, 1 eq) and then the mixture was stirred at 25° C. for 2 hr. The mixture was purified by reversed phase preparative HPLC (45 g) of SepaFlash® Spherical C18, 20-45 um, 100 Å; mobile phase: A for H₂O+0.1% NH3·H₂O and B for acetonitrile; gradient: B 50-60% in 30 min, flow rate: 60 mL/min; column temperature: RT, wavelength: 220 nm/254 nm). The eluent was concentrated to remove organic phase then lyophilized to give I-107-3 (95 mg, crude) as an off-white solid. SFC: R^{t}=0.395 min. F2. ¹H NMR (400 MHZ, DMSO-d₆) δ=11.66 (br s, 1H), 8.31-8.21 (m, 2H), 8.12 (s, 1H), 7.73 (s, 1H), 7.60-7.51 (m, 2H), 7.46-7.44 (m, 1H), 7.32 (s, 4H), 7.20-7.13 (m, 2H), 7.09 (s, 1H), 6.62-6.57 (m, 1H), 4.93-4.78 (m, 1H), 4.69 (s, 2H), 4.24-4.12 (m, 2H), 4.02 (br s, 2H), 3.60-3.58 (m, 6H), 3.47 (br s, 2H), 3.08-3.07 (m, 2H), 2.81-2.73 (m, 2H), 2.46 (s, 3H), 2.40 (br s, 5H), 2.34-2.28 (m, 2H), 2.23 (d, J=1.6 Hz, 3H), 2.13 (d, J=7.2 Hz, 2H), 2.02-1.76 (m, 9H), 1.68-1.66 (m, 2H), 1.42 (br s, 9H), 1.13-1.00 (m, 2H).

### Step 4: Synthesis of I-107

To a solution of I-107-3 (0.08 g, 70.49 μmol, 1 eq) in dioxane (0.4 mL) was added HCl/dioxane (4 M, 0.4 mL, 22.70 eq) and then the mixture was stirred at 25° C. for 0.5 hr. The reaction mixture was combined with another batch. The mixture was concentrated under room temperature reduced pressure to remove solvent to give product. The product was added water (30 mL) and lyophilized to give I-107 (68.74 mg, 64.17 μmol, 91.03% yield, 100% purity, HCl) as an off-white solid. SFC: R^{t}=2.115 min. _F2. ¹H NMR (400 MHz, DMSO-d₆) δ=12.68 (d, J=6.4 Hz, 1H), 11.95-11.45 (m, 2H), 9.93-9.61 (m, 3H), 9.14 (br s, 3H), 8.35 (s, 1H), 8.28 (s, 1H), 7.90 (s, 1H), 7.76 (d, J=2.0 Hz, 1H), 7.72-7.61 (m, 2H), 7.48-7.40 (m, 5H), 6.96 (br s, 1H), 5.07-4.91 (m, 2H), 4.76 (s, 2H), 4.57-4.24 (m, 6H), 4.22-4.06 (m, 4H), 4.03-3.74 (m, 5H), 3.36-3.09 (m, 10H), 2.61-2.53 (m, 2H), 2.45-2.07 (m, 7H), 2.05-1.88 (m, 2H), 1.77-1.52 (m, 2H), 1.23 (br s, 1H). ¹H NMR (400 MHZ, DMSO-d₆) δ=9.67 (s, 1H), 8.36 (s, 1H), 8.25 (t, J=1.6 Hz, 1H), 7.89 (d, J=1.6 Hz, 1H), 7.75-7.66 (m, 2H), 7.65-7.58 (m, 1H), 7.48-7.38 (m, 5H), 6.96 (d, J=3.6 Hz, 1H), 5.12-4.90 (m, 2H), 4.74 (s, 2H), 4.59-4.21 (m, 6H), 4.08-3.77 (m, 5H), 3.64-3.62 (m, 5H), 3.30-2.93 (m, 12H), 2.64-2.53 (m, 2H), 2.47 (br s, 3H), 2.43-2.03 (m, 6H), 2.02-1.81 (m, 2H), 1.80-1.52 (m, 2H), 1.35-1.13 (m, 1H).

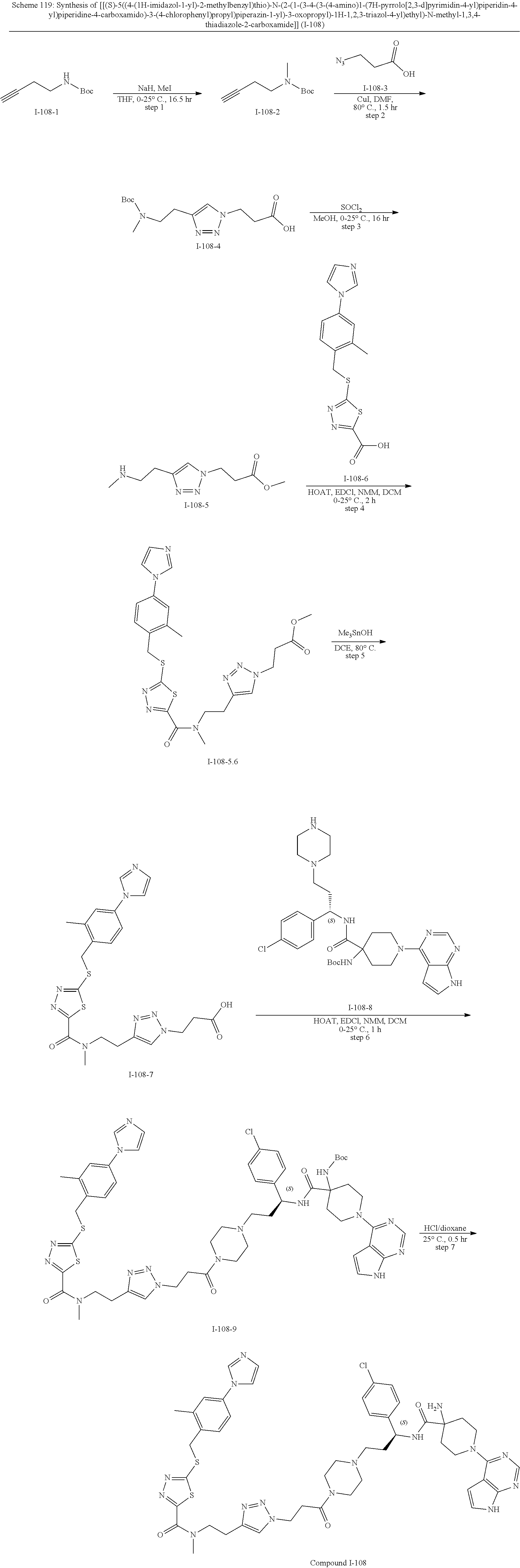

### Step 1: Synthesis of I-108-2

To a solution of I-108-1 (4.9 g, 28.96 mmol, 1 eq) in THF (100 mL) was added NaH (1.74 g, 43.43 mmol, 60% purity, 1.5 eq) in portions at 0-5° C. under N₂, and stirred for 0.5 hr. Then MeI (6.17 g, 43.43 mmol, 2.70 mL, 1.5 eq) was added dropwise. The resulting solution was slowly warmed to 25° C., and stirred for another 16 hrs. The reaction mixture was quenched by added to saturated NH₄Cl aqueous solution (200 mL), extracted with EtOAc (100 mL*2). The combined organic phase was washed with brine (50 mL*2), dried over Na₂SO₄, filtered and concentrated under vacuum to give a yellow oil The residue was purified by flash silica gel chromatography (ISCO®; 80 g SepaFlash® Silica Flash Column, Eluent of 0˜10% Ethyl acetate/Petroleum ether gradient (@ 100 mL/min). The eluent was concentrated under vacuum to give I-108-2 (5.2 g, 28.38 mmol, 98.00% yield, n/a purity) as a colorless oil. ¹H NMR (400 MHZ, CHLOROFORM-d) δ=3.31 (br s, 2H), 2.85 (s, 3H), 2.34 (br s, 2H), 1.90 (t, J=2.4 Hz, 1H), 1.39 (s, 9H).

### Step 2: Synthesis of I-108-4

To a solution of I-108-3 (3.27 g, 28.38 mmol, 1 eq) in DMF (50 mL) was added CuI (540.44 mg, 2.84 mmol, 0.1 eq) and I-108-2 (5.2 g, 28.38 mmol, 1 eq), then the suspension was stirred at 80° C. for 1.5 hr under N₂. The reaction mixture was cooled to 20° C., then poured into brine (100 mL), extracted with EtOAc (70 mL*2). The combined organic phase was washed with brine (50 mL*4), dried over Na₂SO₄, filtered and concentrated under vacuum to give a yellow oil. The residue was purified by flash silica gel chromatography (ISCO®; 80 g SepaFlash® Silica Flash Column, Eluent of 33˜100% Ethyl acetate/Petroleum ether gradient (@ 100 mL/min) to give I-108-4 (7.6 g, 25.47 mmol, 89.77% yield, N/A purity) as a light-yellow oil. ¹H NMR (400 MHZ, CHLOROFORM-d) δ=7.89-7.46 (m, 1H), 6.85-6.55 (m, 1H), 4.70 (t, J=6.4 Hz, 2H), 3.57 (t, J=7.3 Hz, 2H), 3.10-2.96 (m, 4H), 2.94-2.78 (m, 3H), 1.48 (br s, 1H).

### Step 3: Synthesis of I-108-5

To a solution of I-108-4 (1.5 g, 5.03 mmol, 1 eq) in MeOH (15 mL) was added SOCl₂ (1.20 g, 10.06 mmol, 730.36 μL, 2 eq) dropwise at 0° C. under N₂. The mixture was stirred at 25° C. for 16 hr under N₂. The mixture was concentrated under reduced pressure to give I-108-5 (1.3 g, crude, HCl) as a light yellow solid. ¹H NMR (400 MHZ, DMSO-d₆) δ=9.16 (br s, 2H), 8.00 (s, 1H), 4.56 (t, J=6.8 Hz, 2H), 3.60 (s, 3H), 3.15-3.10 (m, 2H), 3.05-2.94 (m, 4H), 2.54 (t, J=5.6 Hz, 3H).

### Step 4: Synthesis of I-108-5.6

To a solution of I-108-5 (1 g, 4.02 mmol, 1 eq, HCl salt), I-108-6 (1.43 g, 4.02 mmol, 1 eq, Na salt), EDCI (2.31 g, 12.06 mmol, 3 eq), HOAt (820.90 mg, 6.03 mmol, 1.5 eq) in DCM (10 mL) was added NMM (4.07 g, 40.21 mmol, 4.42 mL, 10 eq) at 0° C. The mixture was stirred at 25° C. for 2 hr. The reaction mixture was quenched by addition H₂O (10 mL) at 25° C., and extracted with DCM 15 mL (5 mL*3). The combined organic layers were dried over Na₂SO₄, filtered and concentrated under reduced pressure to give a residue. The crude product was purified by reverse-phase HPLC (26.8*125 mm, (220 g) of XB—C18, 20-40 μm, 120 Å) Mobile phase: A for H₂O (0.1% (FA) v/v) and B for acetonitrile; Gradient: B 40%-60% in 10 min: Flow rate: 40 ml/min; Column temperature: R.T. Wavelength: 220 nm/254 nm). The eluent was concentrated and lyophilized to give I-108-5.6 (1.3 g, 2.47 mmol, 61.39% yield, 100% purity) as a yellow solid. LCMS (Method E): R^{t}=0.398 min, [M+H]⁺=527.2. ¹H NMR (400 MHZ, DMSO-d₆) δ=8.25 (s, 1H), 7.94-7.83 (m, 1H), 7.74 (s, 1H), 7.60-7.52 (m, 2H), 7.50-7.44 (m, 1H), 7.09 (s, 1H), 4.69 (d, J=2.0 Hz, 2H), 4.58-4.48 (m, 2H), 4.12 (t, J=7. Hz, 1H), 3.71 (t, J=7.2 Hz, 1H), 3.61-3.56 (m, 3H), 3.35 (s, 1H), 3.03 (s, 2H), 3.01-2.88 (m, 4H), 2.46 (s, 3H).

### Step 5: Synthesis of I-108-7

To a solution of I-108-5.6 (0.1 g, 189.89 μmol, 1 eq) in DCE (1 mL) was added Me₃SnOH (34.34 mg, 189.89 μmol, 1 eq). The mixture was stirred at 80° C. for 18 hr. showed 26% of I-108-5.6 remained. Me₃SnOH (206.01 mg, 1.14 mmol, 6 eq) was added. The mixture was stirred at 80° C. for 2 hr. The reaction mixture was filtered and the filter cake was washed with DCM (0.5 mL*3). The filtrate was concentrated under reduced pressure to give a residue. The residue was purified by flash silica gel chromatography (ISCO®; 4 g SepaFlash® Silica Flash Column, Eluent of 60-70% MeOH/DCM) and the eluent was concentrated to give I-108-7 (0.09 g, 165.04 μmol, 86.91% yield, 94% purity) as yellow oil. LCMS (Method E): R^{t}=0.385 min, [M+H]⁺=513.1. ¹H NMR (400 MHZ, DMSO-d₆) δ=8.25 (s, 1H), 7.97-7.78 (m, 1H), 7.74 (s, 1H), 7.61-7.51 (m, 2H), 7.47-7.45 (m, 1H), 7.09 (s, 1H), 4.69 (d, J=1.6 Hz, 2H), 4.54-4.34 (m, 2H), 4.11 (q, J=5.2 Hz, 2H), 3.71 (t, J=7.2 Hz, 1H), 3.16 (s, 2H), 3.03 (s, 2H), 3.01-2.92 (m, 2H), 2.84-2.82 (m, 2H), 2.46 (s, 3H).

### Step 6: Synthesis of I-108-9

To a mixture of I-108-7 (0.09 g, 175.57 μmol, 1 eq) and I-108-8 (104.84 mg, 175.57 μmol, 1 eq), EDCI (100.97 mg, 526.72 μmol, 3 eq) and NMM (88.80 mg, 877.87 μmol, 96.52 μL, 5 eq) in DMF (0.9 mL) was added HOAt (23.90 mg, 175.57 μmol, 1 eq) and then the mixture was stirred at 25° C. for 1 hr. The reaction mixture was filtered and the filtrate was purified by reversed phase preparative HPLC (45 g) of SepaFlash® Spherical C18, 20-45 μm, 100 Å; mobile phase: A for H₂O+0.1% NH3·H₂O and B for acetonitrile; gradient: B 50-60% in 30 min, flow rate: 60 mL/min; column temperature: RT, wavelength: 220 nm/254 nm) to give I-108-9 (0.1 g. 91.60 μmol, 52.17% yield) as an off-white solid. LCMS (Method G): R^{t}=0.630 min, [M+H]⁺=1091.7. SFC: R^{t}=1.031 min. _G302. ¹H NMR (400 MHZ, DMSO-d₆) δ=11.67 (br s, 1H), 8.25 (s, 2H), 8.13 (s, 1H), 7.94-7.81 (m, 1H), 7.73 (s, 1H), 7.62-7.50 (m, 2H), 7.47-7.45 (m, 1H), 7.31 (s, 4H), 7.24-7.00 (m, 3H), 6.60 (br s, 1H), 4.87-4.86 (m, 1H), 4.69 (s, 2H), 4.51-4.49 (m, 2H), 4.21-4.19 (m, 2H), 4.12 (t, J=6.8 Hz, 1H), 3.72 (t, J=7.6 Hz, 1H), 3.60-3.56 (m, 2H), 3.52-3.40 (m, 4H), 3.37 (s, 2H), 3.04 (s, 2H), 3.02-2.89 (m, 4H), 2.46 (s, 3H), 2.37-2.18 (m, 6H), 2.02-1.74 (m, 6H), 1.41 (br s, 9H).

### Step 7: Synthesis of I-108

To a solution of I-108-9 (0.1 g, 91.60 μmol, 1 eq) in dioxane (0.5 mL) was added HCl/dioxane (4 M, 0.5 mL) and then the mixture was stirred at 25° C. for 0.5 hr. The reaction mixture was concentrated (room temperature) under reduced pressure to remove solvent and give I-108 (69.58 mg, 67.34 μmol, 73.52% yield, 99.498% purity, HCl) as an off-white solid. LCMS (Method E): R^{t}=0.384 min, [M+H]⁺=991.4. SFC: R^{t}=1.802 min. _f301. ¹H NMR (400 MHZ, DMSO+D2O) δ=9.69 (s, 1H), 8.40 (s, 1H), 8.25 (s, 1H), 7.94-7.80 (m, 2H), 7.72-7.70 (m, 1H), 7.71-7.66 (m, 1H), 7.65-7.59 (m, 1H), 7.48 (d, J=3.2 Hz, 1H), 7.46-7.35 (m, 4H), 7.01 (d, J=3.6 Hz, 1H), 4.93-4.91 (m, 1H), 4.72 (d, J=3.2 Hz, 2H), 4.50-4.47 (m, 2H), 4.41-4.23 (m, 3H), 4.14-3.96 (m, 4H), 3.57-3.45 (m, 7H), 3.35 (s, 2H), 3.26-3.06 (m, 4H), 3.02 (s, 2H), 3.00-2.90 (m, 4H), 2.68-2.56 (m, 2H), 2.48 (br s, 3H), 2.39-2.29 (m, 1H), 2.25-2.10 (m, 2H), 2.05-1.93 (m, 1H).

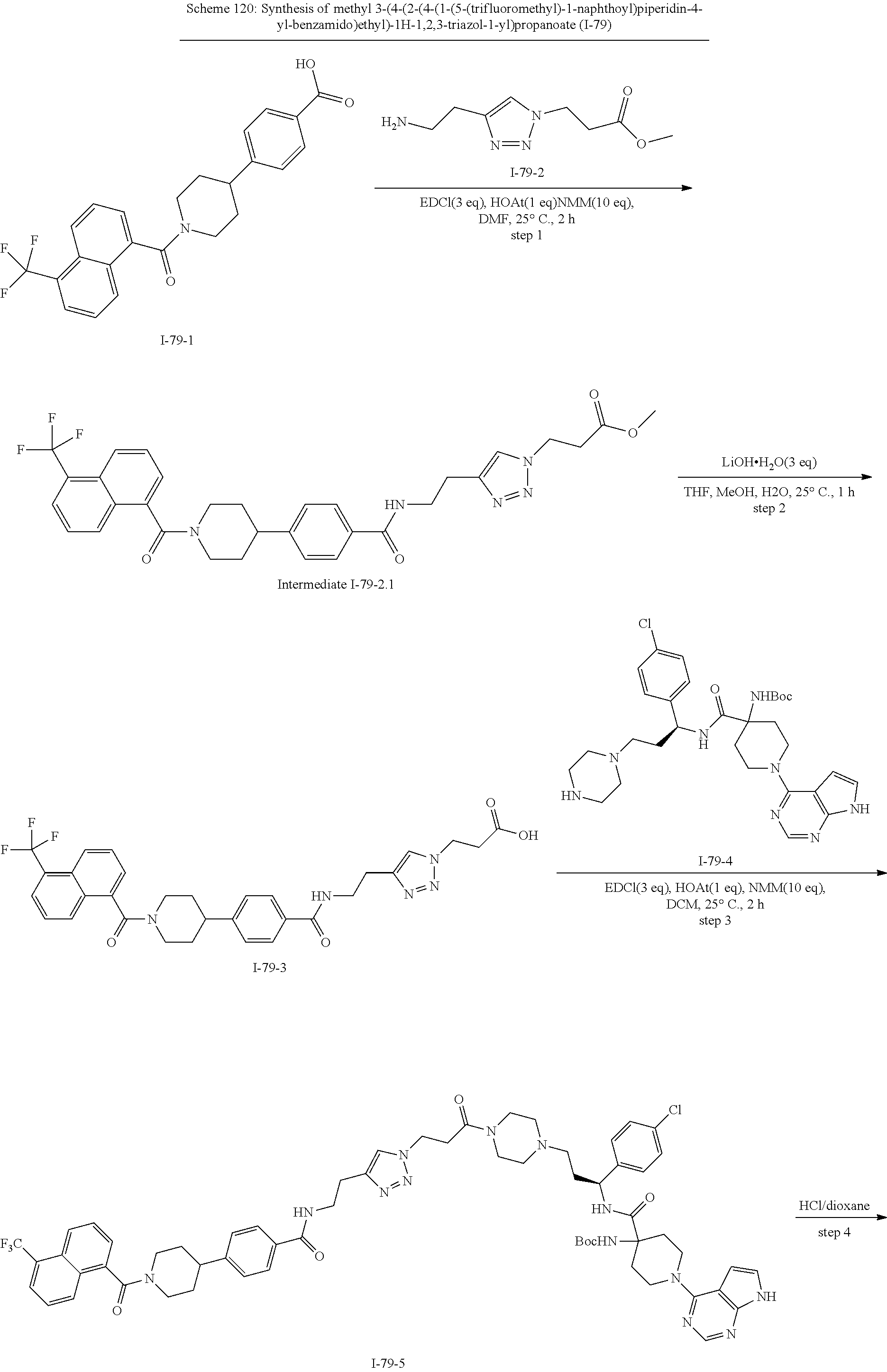

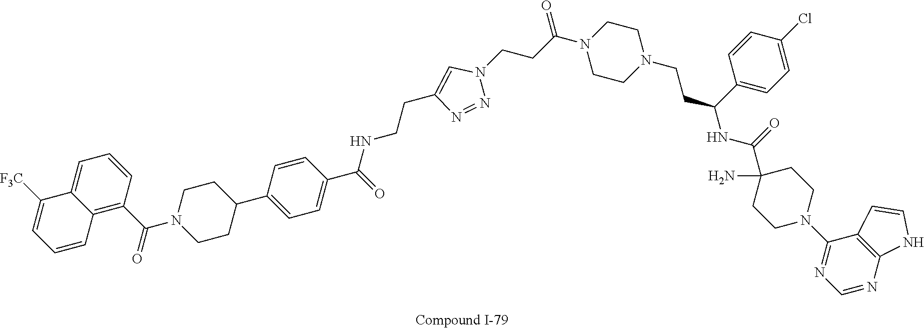 [SMILES: NC1(C(=O)N[C@@H](CCN2CCN(C(=O)CCn3cc(CCNC(=O)c4ccc(C5CCN(C(=O)c6cccc7c(C(F)(F)F)cccc67)CC5)cc4)nn3)CC2)c2ccc(Cl)cc2)CCN(c2ncnc3[nH]ccc23)CC1]

### Step 1: Synthesis of I-79-2.1

To a solution of I-79-1 (100 mg, 233.96 μmol, 1 eq) and 1-2 (65.89 mg, 280.76 μmol, 1.2 eq, HCl) in DCM (1 mL) was added EDCI (134.55 mg, 701.89 μmol, 3 eq), HOAt (31.85 mg, 233.96 μmol, 32.73 μL, 1 eq) and NMM (236.65 mg, 2.34 mmol, 257.23 μL, 10 eq). The mixture was stirred at 25° C. for 2 h. The reaction mixture was quenched by addition H₂O 1 mL, and then diluted with DCM 1 mL and extracted with DCM 6 mL (2 mL*3). The combined organic layers were dried over anhydrous Na₂SO₄, filtered and concentrated under reduced pressure to give a residue. The residue was purified by prep-HPLC (column: YMC-Actus Triart C18 150*30 mm*7 um; mobile phase: [water (FA)-ACN]; gradient: 45%-75% B over 10 min), then concentrated to remove organic solvents and lyophilized to give product. I-79-2.1 (50 mg, 82.29 μmol, 35.17% yield, 100% purity) was obtained as a white solid. LCMS (Method E): R^{t}=0.532 min, [M+H]⁺=608.3. ¹H NMR (400 MHZ, METHANOL-d4) δ=8.28-7.99 (m, 3H), 7.81-7.57 (m, 6H), 7.39-7.34 (m, 2H), 5.07-4.95 (m, 1H), 4.65-4.62 (m, 2H), 3.69-3.56 (m, 5H), 3.51-3.39 (m, 1H), 3.27-3.05 (m, 2H), 3.03-2.90 (m, 5H), 2.12-1.82 (m, 2H), 1.76-1.41 (m, 2H), 19FNMR: (377 MHz, METHANOL-d4).

### Step 2: Synthesis of I-79-2

To a solution of I-79-2.1 (100 mg, 164.58 μmol, 1 eq) in MeOH (1 mL), THF (1 mL), H₂O (0.5 mL) was added LiOH·H₂O (20.72 mg, 493.73 μmol, 3 eq). The mixture was stirred at 25° C. for 0.5 h. The reaction mixture was quenched by addition H₂O 1 mL, then adjust pH=6 with citric acid, diluted with EA 1 mL and extracted with EA 6 mL (2 mL*3). The combined organic layers were dried over anhydrous Na₂SO₄, filtered, and concentrated under reduced pressure to give I-79-2 (100 mg, crude) as a white solid. LCMS (Method E): R^{t}=0.515 min, [M+H]⁺=594.3.

### Step 3: Synthesis of I-79-3

To a solution of I-79-2 (90 mg, 151.62 μmol, 1 eq) and I-79-2 (99.59 mg, 166.78 μmol, 1.1 eq) in DCM (2 mL) was added EDCI (87.20 mg, 454.86 μmol, 3 eq), HOAt (20.64 mg, 151.62 μmol, 21.21 μL, 1 eq) and NMM (153.36 mg, 1.52 mmol, 166.69 μL, 10 eq). The mixture was stirred at 25° C. for 2 h. The reaction mixture was quenched by addition H₂O 1 mL, and then diluted with DCM 1 mL and extracted with DCM 6 mL (2 mL*3). The combined organic layers were dried over anhydrous Na₂SO₄, filtered and concentrated under reduced pressure to give I-79-3 (160 mg, crude) as a yellow solid, and used into next step without further purification. LCMS (Method G): R^{t}=0.705 min, [M+H]⁺=1172.8.

### Step 4: Synthesis of I-79

To a solution of I-79-3 (150 mg, 127.91 μmol, 1 eq) in DCM (1.5 mL) was added HCl/dioxane (4 M, 31.98 μL, 1 eq). The mixture was stirred at 25° C. for 0.5 h. Upon completion, the reaction mixture was concentrated under reduced pressure to give a residue, then purified by prep-HPLC (column: Phenomenex luna C18 150*25 mm*10 um; mobile phase: [water (FA)-ACN]; gradient: 17%-47% B over min) and concentrated to remove organic solvents and lyophilized to give I-79 (39.75 mg, 35.53 μmol, 27.78% yield, 100% purity, FA) as a white solid. LCMS (Method E): R^{t}=0.479 min, [M+H]⁺=1072.7. ¹H NMR (400 MHz, METHANOL-d4) δ=8.56-8.45 (m, 1H), 8.28-8.00 (m, 4H), 7.82-7.56 (m, 6H), 7.39-7.31 (m, 6H), 7.20 (d, J=3.6 Hz. 1H), 6.70 (d, J=2.4 Hz. 1H), 5.10-4.94 (m, 3H), 4.78-4.60 (m, 5H), 3.72-3.54 (m, 7H), 3.45-3.40 (m, 1H), 3.24-3.17 (m, 1H), 3.06-2.85 (m, 10H), 2.54-2.41 (m, 2H), 2.21 (s, 2H). 2.12-2.03 (m, 2H), 1.85 (d, J=13.6 Hz, 2H), 1.77-1.28 (m, 3H). SFC: R^{t}=2.408 min. ee value=100%. F3.

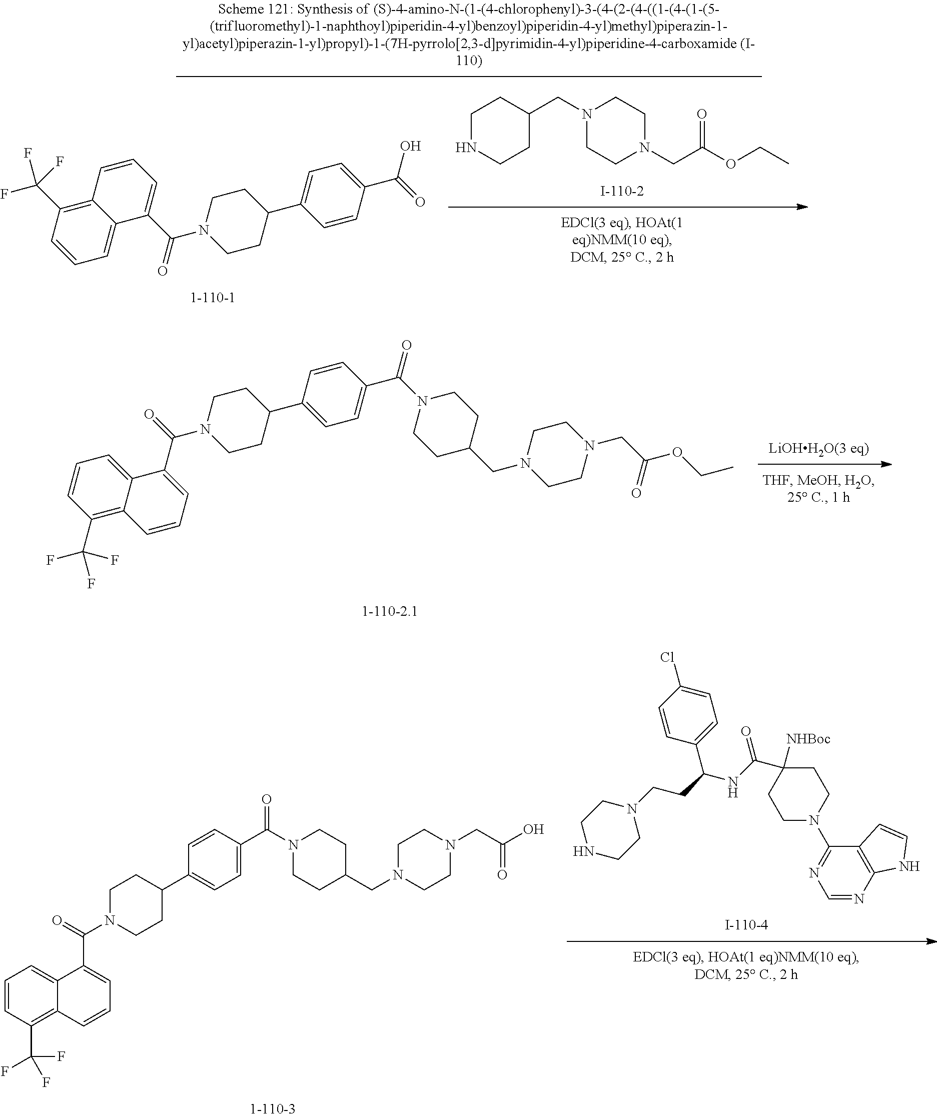 [SMILES: O=C(O)c1ccc(C2CCN(C(=O)c3cccc4c(C(F)(F)F)cccc34)CC2)cc1 ; CCOC(=O)CN1CCN(CC2CCNCC2)CC1 ; CCOC(=O)CN1CCN(CC2CCN(C(=O)c3ccc(C4CCN(C(=O)c5cccc6c(C(F)(F)F)cccc56)CC4)cc3)CC2)CC1 ; O=C(O)CN1CCN(CC2CCN(C(=O)c3ccc(C4CCN(C(=O)c5cccc6c(C(F)(F)F)cccc56)CC4)cc3)CC2)CC1 ; CC(C)(C)OC(=O)NC1(C(=O)N[C@@H](CCN2CCNCC2)c2ccc(Cl)cc2)CCN(c2ncnc3[nH]ccc23)CC1]

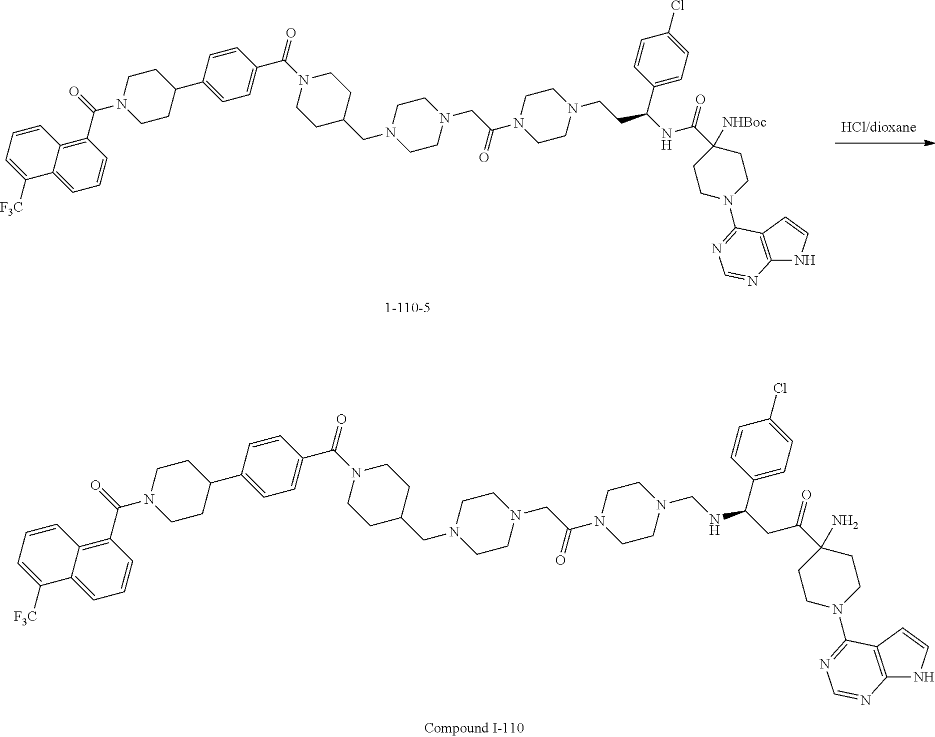 [SMILES: CC(C)(C)OC(=O)NC1(C(=O)N[C@@H](CCN2CCN(C(=O)CN3CCN(CC4CCN(C(=O)c5ccc(C6CCN(C(=O)c7cccc8c(C(F)(F)F)cccc78)CC6)cc5)CC4)CC3)CC2)c2ccc(Cl)cc2)CCN(c2ncnc3[nH]ccc23)CC1 ; NC1(C(=O)C[C@@H](NCN2CCN(C(=O)CN3CCN(CC4CCN(C(=O)c5ccc(C6CCN(C(=O)c7cccc8c(C(F)(F)F)cccc78)CC6)cc5)CC4)CC3)CC2)c2ccc(Cl)cc2)CCN(c2ncnc3[nH]ccc23)CC1]

### Step 1: Synthesis of I-110-2.1

To a solution of I-110-1 (100 mg, 233.96 μmol, 1 eq) and I-110-2 (85.87 mg, 280.76 μmol, 1.2 eq, HCl) in DCM (1 mL) was added EDCI (134.55 mg, 701.89 μmol, 3 eq), HOAt (31.85 mg, 233.96 μmol, 32.73 μL, 1 eq) and NMM (236.65 mg, 2.34 mmol, 257.23 μL, 10 eq). The mixture was stirred at 25° C. for 2 h. Upon completion, the reaction mixture was quenched by addition H₂O 1 mL, and then diluted with DCM 1 mL and extracted with DCM 6 mL (2 mL*3). The combined organic layers were dried over anhydrous Na₂SO₄, filtered and concentrated under reduced pressure to give a residue. The residue was purified by prep-HPLC (column: YMC-Actus Triart C18 150*30 mm*7 um; mobile phase: [water (FA)-ACN]; gradient: 25%-55% B over 10 min), then concentrated to remove organic solvents and lyophilized to give I-110-2.1 (50 mg, 73.43 μmol, 31.39% yield, 99.69% purity) as a yellow solid. LCMS (Method E): R^{t}=0.490 min, (M+H)=679.4. ¹H NMR (400 MHZ, METHANOL-d4) δ=8.28-8.00 (m, 3H), 7.79-7.57 (m, 3H), 7.41-7.32 (m, 4H), 5.06-4.97 (m, 1H), 4.69-4.53 (m, 1H), 4.20-4.15 (m, 2H), 3.85-3.66 (m, 1H), 3.48-3.38 (m, 1H), 3.30-3.25 (m, 2H), 3.24-3.03 (m, 3H), 3.00-2.62 (m, 10H), 2.44 (d, J=5.6 Hz, 2H), 2.13-1.65 (m, 7H), 1.31-1.06 (m, 5H).

### Step 2: Synthesis of I-110-3

To a solution of I-110-2.1 (100 mg, 147.32 μmol, 1 eq) in THF (1 mL), H₂O (0.5 mL), MeOH (1 mL) was added LiOH·H₂O (18.55 mg, 441.97 μmol, 3 eq). The mixture was stirred at 25° C. for 1 h. The reaction mixture was adjust pH=7 with HCl (1M) solution, and then concentrated under reduced pressure to give a residue. The residue was purified by prep-HPLC (column: Phenomenex Luna C18 150*25 mm*10 um; mobile phase: [water (NH4HCO3)-ACN]; gradient: 24%-54% B over 10 min), then concentrated to remove organic solvents and lyophilized to give I-110-3 (90 mg, 138.31 μmol, 93.88% yield, N/A purity) as a white solid. LCMS (Method G): R^{t}=0.421 min, (M+H)=651.4.

### Step 3: Synthesis of I-110-5

To a solution of I-110-3 (70 mg, 107.57 μmol, 1 eq) and I-110-4 (70.66 mg, 118.33 μmol, 1.1 eq) in DCM (1 mL) was added EDCI (61.87 mg, 322.72 μmol, 3 eq), HOAt (14.64 mg, 107.57 μmol, 15.05 μL, 1 eq) and NMM (108.81 mg, 1.08 mmol, 118.27 μL, 10 eq). The mixture was stirred at 25° C. for 2 h. The reaction mixture was quenched by addition H₂O 1 mL, and then diluted with DCM 1 mL and extracted with DCM 6 mL (2 mL*3). The combined organic layers were dried over anhydrous Na₂SO₄, filtered and concentrated under reduced pressure to give I-110-5 (130 mg, crude) as a yellow solid. LCMS (Method G): R^{t}=0.739 min, (M+H)=1229.8.

### Step 4: Synthesis of I-110

To a solution of I-110-5 (120 mg, 97.57 μmol, 1 eq) in DCM (1 mL) was added HCl/dioxane (4 M, 24.39 μL, 1 eq). The mixture was stirred at 25° C. for 1 h. The reaction mixture was concentrated under reduced pressure to give a residue. The residue was purified by prep-HPLC (column: Phenomenex Luna C18 150*25 mm*10 um; mobile phase: [water (FA)-ACN]; gradient: 10%-40% B over min), then concentrated to remove organic solvents and lyophilized to give I-110 (61.21 mg, 52.06 μmol, 53.35% yield, 100% purity, FA) as a white solid. LCMS (Method E): R^{t}=0.469 min, (M+H)=1129.9. ¹H NMR (400 MHZ, METHANOL-d4) δ=8.42 (s, 1H), 8.27-8.00 (m, 4H), 7.79-7.57 (m, 3H), 7.41-7.29 (m, 8H), 7.15 (d, J=3.6 Hz, 1H), 6.64 (d, J=3.2 Hz, 1H), 5.05-4.96 (m, 2H), 4.59 (br s, 3H), 3.78-3.39 (m, 10H), 3.27-3.05 (m, 3H), 3.01-2.70 (m, 10H), 2.58-2.34 (m, 8H), 2.27 (d, J=9.6 Hz, 2H), 2.10-1.84 (m, 6H), 1.79-1.40 (m, 5H), 1.17 (s, 2H). SFC: R^{t}=1.116 min, ee value=100%. F301.

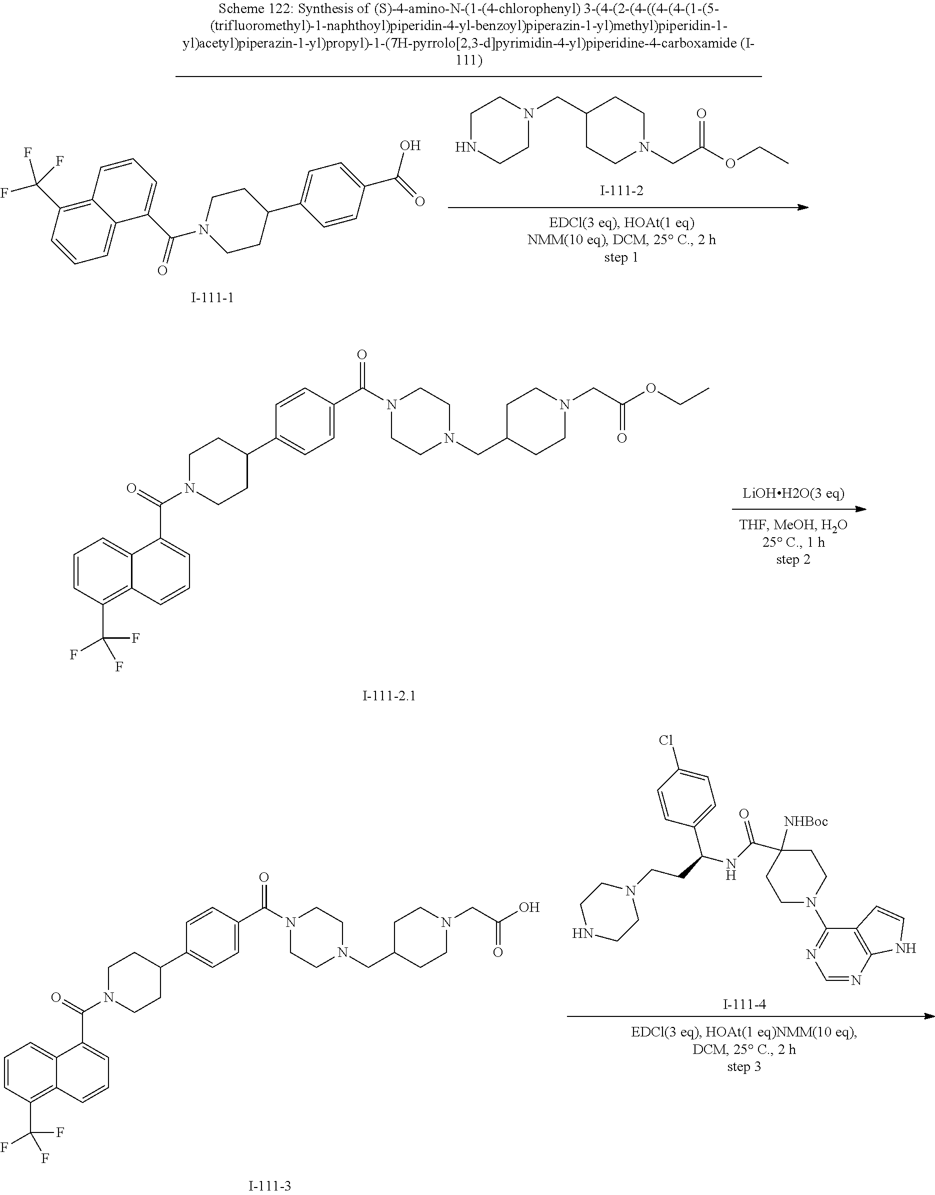

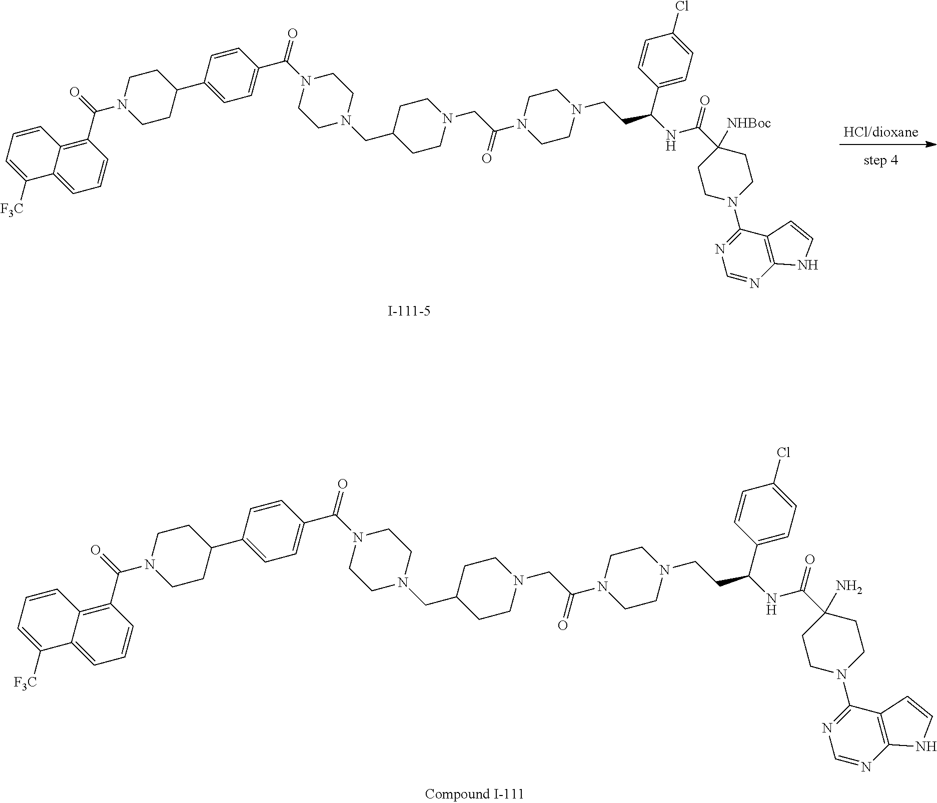 [SMILES: CC(C)(C)OC(=O)NC1(C(=O)N[C@@H](CCN2CCN(C(=O)CN3CCC(CN4CCN(C(=O)c5ccc(C6CCN(C(=O)c7cccc8c(C(F)(F)F)cccc78)CC6)cc5)CC4)CC3)CC2)c2ccc(Cl)cc2)CCN(c2ncnc3[nH]ccc23)CC1 ; "HCl/dioxane" ; NC1(C(=O)N[C@@H](CCN2CCN(C(=O)CN3CCC(CN4CCN(C(=O)c5ccc(C6CCN(C(=O)c7cccc8c(C(F)(F)F)cccc78)CC6)cc5)CC4)CC3)CC2)c2ccc(Cl)cc2)CCN(c2ncnc3[nH]ccc23)CC1]

### Step 1: Synthesis of I-111-2.1

To a solution of I-111-1 (100 mg, 233.96 μmol, 1 eq) and I-111-2 (85.87 mg, 280.76 μmol, 1.2 eq. HCl) in DCM (1 mL) was added EDCI (134.55 mg, 701.89 μmol, 3 eq), HOAt (31.85 mg, 233.96 μmol, 32.73 μL, 1 eq) and NMM (236.65 mg, 2.34 mmol, 257.23 μL, 10 eq). The mixture was stirred at 25° C. for 2 h. The reaction mixture was quenched by addition H₂O 1 mL, and then diluted with DCM 1 mL and extracted with DCM 6 mL (2 mL*3). The combined organic layers were dried over anhydrous Na₂SO₄, filtered and concentrated under reduced pressure to give a residue. The residue was purified by prep-HPLC (column: YMC-Actus Triart C18 150*30 mm*7 um; mobile phase: [water (FA)-ACN]; gradient: 18%-48% B over 10 min), then concentrated to remove organic solvents and lyophilized to give I-111-2.1 (50 mg, 68.98 μmol, 29.48% yield, 100% purity, FA) as a white solid. LCMS (Method E): R^{t}=0.447 min, [M+H]⁺=679.4. ¹H NMR (400 MHZ, METHANOL-d4) δ=8.43 (s, 1H), 8.29-7.99 (m, 3H), 7.80-7.56 (m, 3H), 7.43-7.33 (m, 4H), 5.05-4.98 (m, 1H), 4.24-4.19 (m, 2H), 3.74 (s, 2H), 3.57-3.38 (m, 5H), 3.30-3.15 (m, 3H), 3.14-2.88 (m, 2H), 2.63-2.36 (m, 6H), 2.29 (d, J=7.2 Hz, 2H), 2.12-1.80 (m, 4H), 1.78-1.43 (m, 3H), 1.42-1.32 (m, 2H), 1.30-1.26 (m, 3H).

### Step 2: Synthesis of I-111-3

To a solution of I-111-2.1 (100 mg, 147.32 μmol, 1 eq) in THF (1 mL), H₂O (0.5 mL), MeOH (1 mL) was added LiOH·H₂O (18.54 mg, 441.97 μmol, 3 eq). The mixture was stirred at 25° C. for 1 h. The reaction mixture was adjust pH=7 with HCl (1M) solution, and then concentrated under reduced pressure to give a residue. The residue was purified by prep-HPLC (column: Phenomenex Luna C18 150*25 mm*10 um; mobile phase: [water (NH4HCO3)-ACN]; gradient: 18%-48% B over 10 min), then concentrated to remove organic solvents and lyophilized to give I-111-3 (90 mg, 138.31 μmol, 93.88% yield, N/A purity) as a white solid. LCMS (Method G): R^{t}=0.494 min, [M+H]⁺=651.4.

### Step 3: Synthesis of I-111-5

To a solution of I-111-3 (60 mg, 92.20 μmol, 1 eq) I-111-4 (60.57 mg, 101.42 μmol, 1.1 eq) in DCM (1 mL) was added EDCI (53.03 mg, 276.61 μmol, 3 eq), HOAt (12.55 mg, 92.20 μmol, 12.90 μL, 1 eq) NMM (93.26 mg, 922.04 μmol, 101.37 μL, 10 eq). The mixture was stirred at 25° C. for 2 h. The reaction mixture was quenched by addition H₂O 1 mL, and then diluted with DCM 1 mL and extracted with DCM 6 mL (2 mL*3). The combined organic layers were dried over anhydrous Na₂SO₄, filtered and concentrated under reduced pressure to give I-111-5 (110 mg, crude) as a yellow solid. LCMS (Method G): R^{t}=0.765 min, [M+H]⁺=1229.8.

### Step 4: Synthesis of I-111

To a solution of I-111-5 (100 mg, 81.31 μmol, 1 eq) in DCM (1 mL) was added HCl/dioxane (4 M, 1 mL, 49.19 eq). The mixture was stirred at 25° C. for 1 h. The reaction mixture was concentrated under reduced pressure to give a residue. The residue was purified by prep-HPLC (column: Phenomenex luna C18 150*25 mm*10 um; mobile phase: [water (FA)-ACN]; gradient: 6%-36% B over min), then concentrated to remove organic solvents and lyophilized to give I-111 (45.47 mg, 38.67 μmol, 47.56% yield, 100% purity, FA) as a white solid. LCMS (Method E): R^{t}=0.468 min, [M+H]⁺=1129.9. ¹H NMR (400 MHZ, METHANOL-d4) δ=8.51-8.37 (m, 1H), 8.29-8.00 (m, 4H), 7.79-7.57 (m, 3H), 7.43-7.26 (m, 8H), 7.15 (d, J=3.6 Hz, 1H), 6.64 (d, J=3.6 Hz, 1H), 5.07-4.96 (m, 2H), 4.56 (d, J=5.4 Hz, 2H), 3.98 (s, 2H), 3.83-3.58 (m, 6H), 3.44 (s, 7H), 3.27-3.06 (m, 2H), 3.00-2.78 (m, 3H), 2.59-2.18 (m, 14H), 2.11-1.80 (m, 7H), 1.72 (d, J=10.4 Hz, 3H), 1.59-1.20 (m, 3H), 19FNMR: (377 MHz, METHANOL-d4). SFC: R^{t}=0.898 min, ee value=100%.

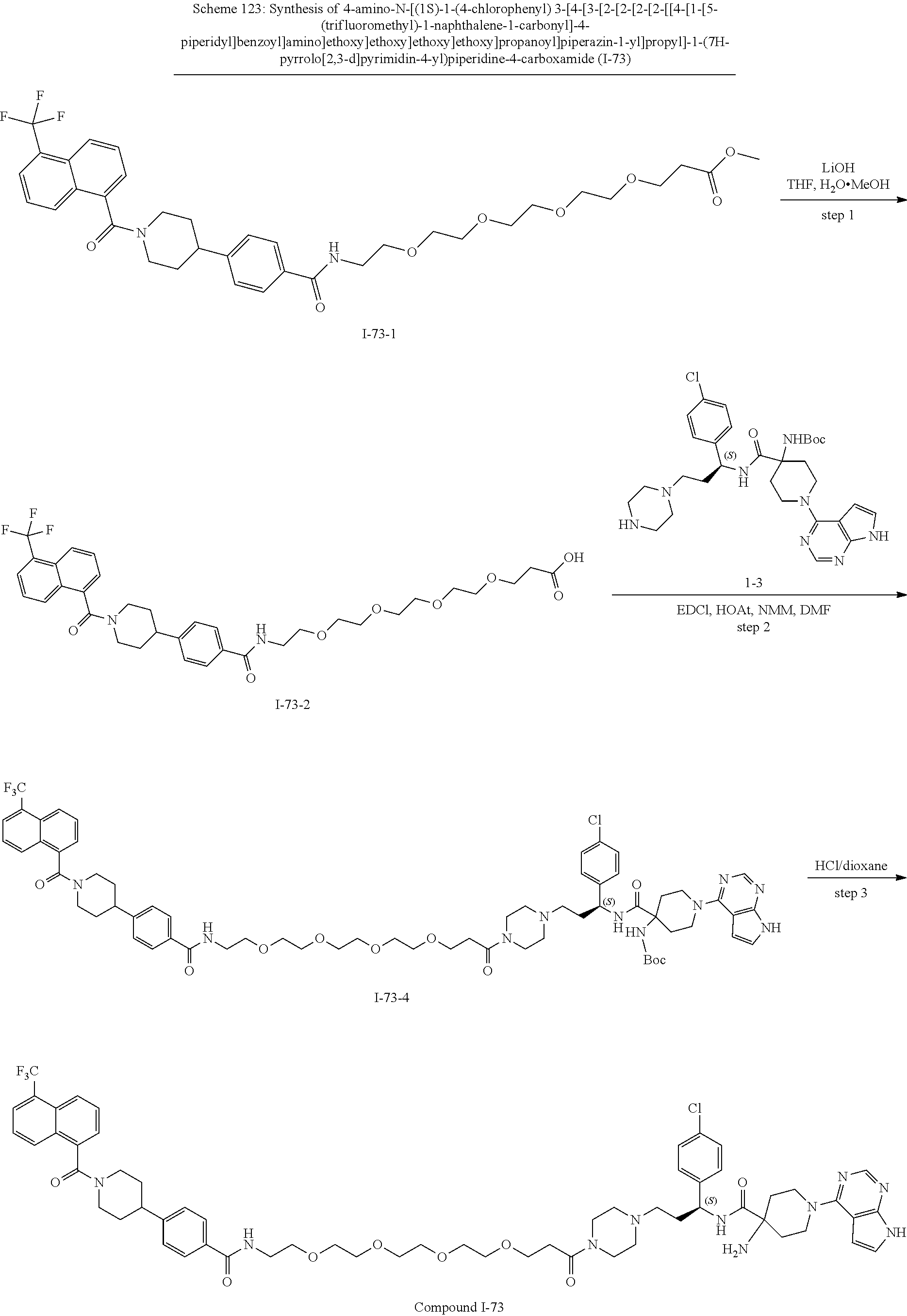

### Step 1: Synthesis of I-73-2

To a solution of I-73-1 (100 mg, 145.20 μmol, 1 eq) in THF (1 mL), H₂O (0.5 mL) and MeOH (0.5 mL) was added LiOH (10.43 mg, 435.59 μmol, 3 eq). The mixture was stirred at 25° C. for 1 hr. The mixture was poured into water 10 mL and extracted with EA (10 mL*3). The combined organic layers were concentrated to give I-73-2 (87 mg, 126.43 μmol, 87.08% yield, 98.05% purity) as a brown solid. LCMS (Method E): R^{t}=0.523 min, [M+H]⁺=675.3. ¹H NMR (400 MHZ, METHANOL-d4) δ=8.30-7.99 (m, 3H), 7.84-7.55 (m, 5H), 7.38-7.30 (m, 2H), 5.07-4.98 (m, 1H), 3.73-3.53 (m, 18H), 3.49-3.40 (m, 1H), 3.25-3.05 (m, 2H), 3.01-2.91 (m, 1H), 2.55-2.45 (m, 2H), 1.97-1.85 (m, 1H), 1.75-1.68 (m, 1H), 1.29 (s, 2H).

### Step 2: Synthesis of I-73-4

To a solution of I-73-2 (77 mg, 114.12 μmol, 1 eq) and I-73-3 (74.96 mg, 125.54 μmol, 1.1 eq) in DMF (1 mL) was added EDCI (65.63 mg, 342.37 μmol, 3 eq), HOAt (15.53 mg, 114.12 μmol, 15.96 μL, 1 eq) and NMM (115.43 mg, 1.14 mmol, 125.47 μL, 10 eq). The mixture was stirred at 25° C. for 1 hr. The mixture was poured into water 10 mL and extracted with EA (10 mL*3). The combined organic layers were concentrated to give a residue. The product was purified by prep-HPLC (column: YMC-Actus Triart C18 150*30 mm*7 um; mobile phase: [water (FA)-ACN]; gradient: 28%-58% B over 10 min) and the eluent was concentrated to remove MeCN and then lyophilization. I-73-4 (65 mg, 51.84 μmol, 45.42% yield, 100% purity) was obtained as a brown solid. LCMS (Method E): R^{t}=0.532 min, [M+H]⁺=1253.6.

### Step 3: Synthesis of I-73

To a solution of I-73-4 (60 mg, 47.85 μmol, 1 eq) in dioxane (1 mL) was added HCl/dioxane (4 M, 1 mL, 83.59 eq). The mixture was stirred at 25° C. for 0.5 hr. The reaction mixture was concentrated under reduced pressure, then was added H₂O to lyophilization to give I-73 (51.38 mg, 43.10 μmol, 90.07% yield, 99.84% purity, HCl) as a white solid. LCMS (Method E): R^{t}=0.471 min, [M+H]⁺=1153.8. SFC: R^{t}=1.085 min, ee value=100%. ¹H NMR (400 MHZ, METHANOL-d4) δ=8.37 (s, 1H), 8.29-7.99 (m, 3H), 7.80-7.58 (m, 5H), 7.45-7.31 (m, 7H), 6.99 (d, J=3.6 Hz, 1H), 5.08-4.95 (m, 2H), 4.70-4.55 (m, 3H), 4.31-4.16 (m, 1H), 4.02-3.87 (m, 2H), 3.74-3.68 (m, 2H), 3.67-3.39 (m, 21H), 3.23-3.02 (m, 5H), 3.02-2.91 (m, 2H), 2.90-2.71 (m, 3H), 2.61-2.43 (m, 2H), 2.37-2.17 (m, 2H), 2.12-1.99 (m, 2H), 1.98-1.82 (m, 1H), 1.74-1.68 (m, 1H), 1.52-1.29 (m, 1H). ¹⁹F NMR (377 MHz, METHANOL-d4) δ=−60.977.

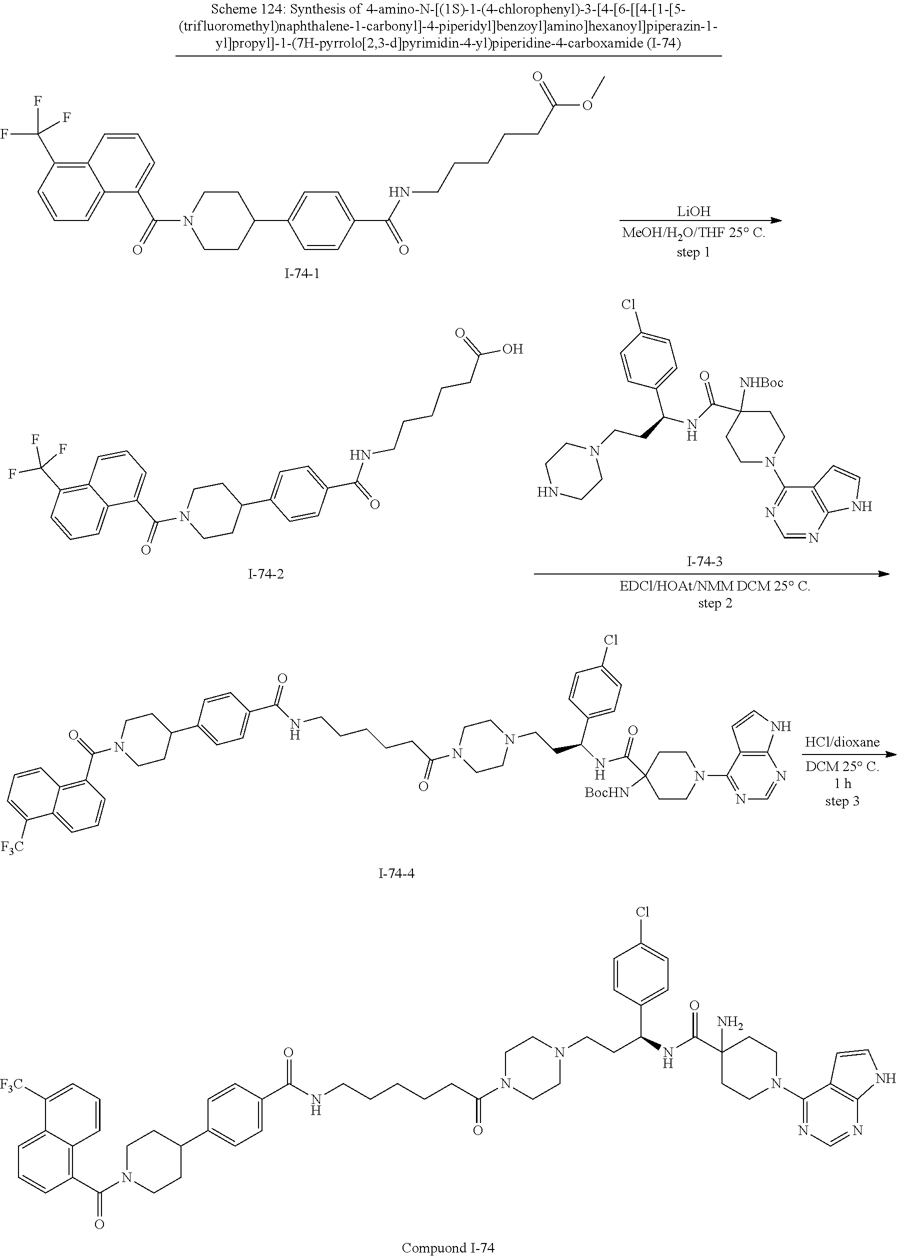

### Step 1: Synthesis of I-74-2

To a solution of I-74-1 (100 mg, 180.31 μmol, 1 eq) in MeOH (1 mL), THF (1 mL) and H₂O (1 mL) was added LiOH·H₂O (22.70 mg, 540.93 μmol, 3 eq). The mixture was stirred at 25° C. for 1 hr. The reaction was concentrated under reduced pressure to give a residue. Then water phase was washed with saturated aqueous citric acid adjusted pH=5, extracted with EA 12 mL (4 mL*3). Then dried over Na₂SO₄, filtered and concentrated under reduced pressure to give I-74-2 (100 mg, crude) as a white solid. LCMS (Method E): R^{t}=0.544 min, [M+H]⁺=541.3.

### Step 2: Synthesis of I-74-4

To a solution of I-74-2 (100 mg, 184.99 μmol, 1 eq) and I-74-3 (110.47 mg, 184.99 μmol, 1 eq) in DCM (2 mL) was added EDCI (106.39 mg, 554.97 μmol, 3 eq), NMM (187.11 mg, 1.85 mmol, 203.38 μL, 10 eq) and HOAt (37.77 mg, 277.48 μmol, 38.82 μL, 1.5 eq). The mixture was stirred at 25° C. for 1 hr. The reaction was cooled to room temperature, which was diluted with H₂O (3 mL), extracted with DCM 15 mL (5 mL*3), the organic phase was washed with saturated aqueous NaCl (5 mL). Then dried over Na₂SO₄, filtered and concentrated under reduced pressure to give I-74-4 (220 mg, crude) as a yellow solid. LCMS (Method E): R^{t}=0.519 min, [M+H]⁺=1119.5.

### Step 3: Synthesis of I-74

The mixture of I-74-4 (200 mg, 178.62 μmol, 1 eq) in DCM (1 mL) and HCl/dioxane (0.2 mL) was stirred at 25° C. for 1 hr. The reaction was filtered and concentrated under reduced pressure to give a residue.

The residue was purified by prep-HPLC (column: Waters Xbridge 150*25 mm*5 um; mobile phase: [water (NH₄HCO₃)-ACN]; gradient: 42%-72% B over 9 min). The eluent was concentrated under reduced pressure to remove ACN, then lyophilized to afford I-74 (90 mg, 88.27 μmol, 49.42% yield, 100% purity) as a white solid. LCMS (Method G): R^{t}=0.672 min, [M+H]⁺=1019.6. SFC: R^{t}=0.693 min. ¹H NMR (400 MHz, METHANOL-d4) δ=8.35-7.94 (m, 4H), 7.80-7.55 (m, 5H), 7.41-7.27 (m, 6H), 7.11 (d, J=3.6 Hz, 1H), 6.61 (d, J=3.6 Hz, 1H), 5.04-4.94 (m, 2H), 4.54-4.44 (m, 2H), 3.68-3.51 (m, 6H), 3.45-3.36 (m, 3H), 3.26-3.02 (m, 2H), 2.99-2.87 (m, 1H), 2.52-2.27 (m, 8H), 2.25-2.10 (m, 2H), 2.00 (br s, 2H), 1.99-1.80 (m, 2H), 1.73-1.37 (m, 10H).

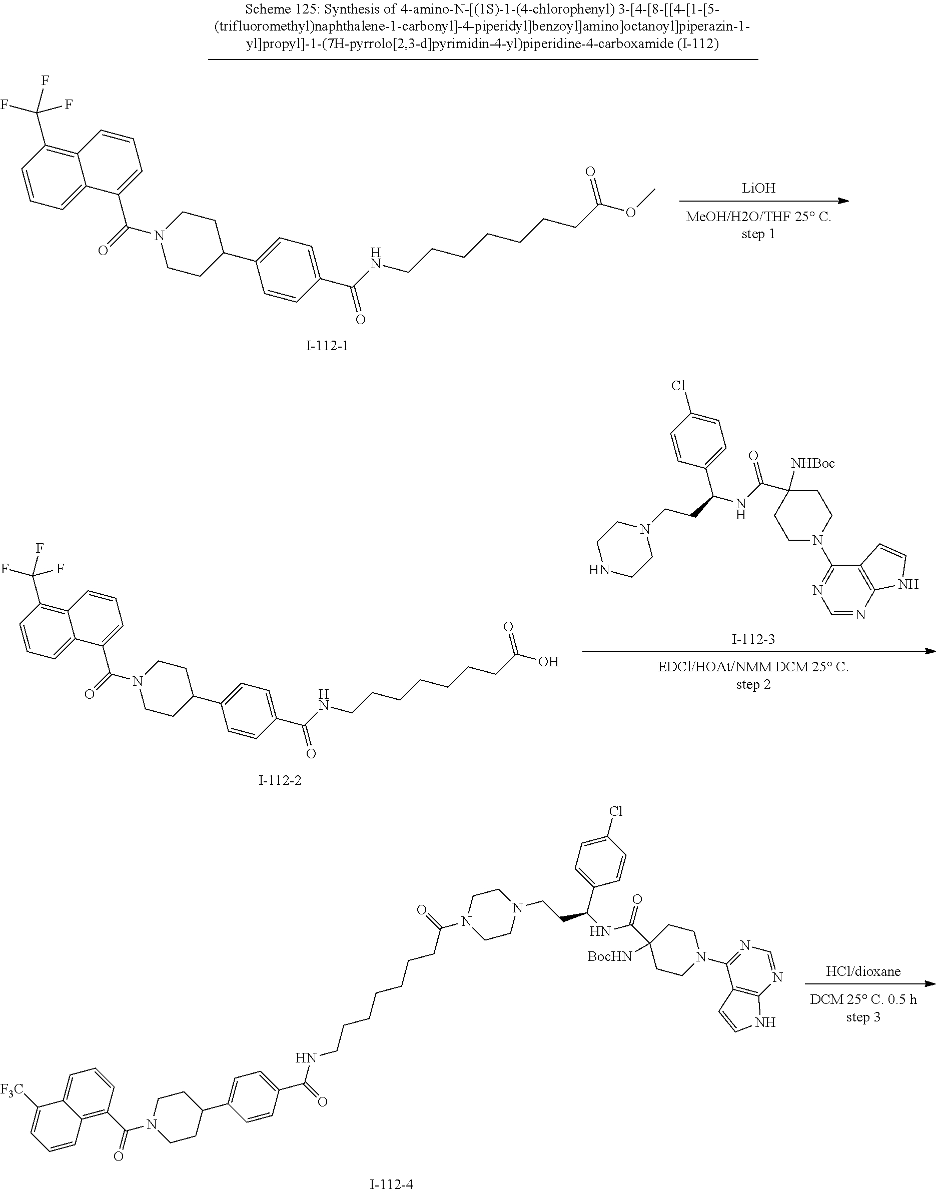

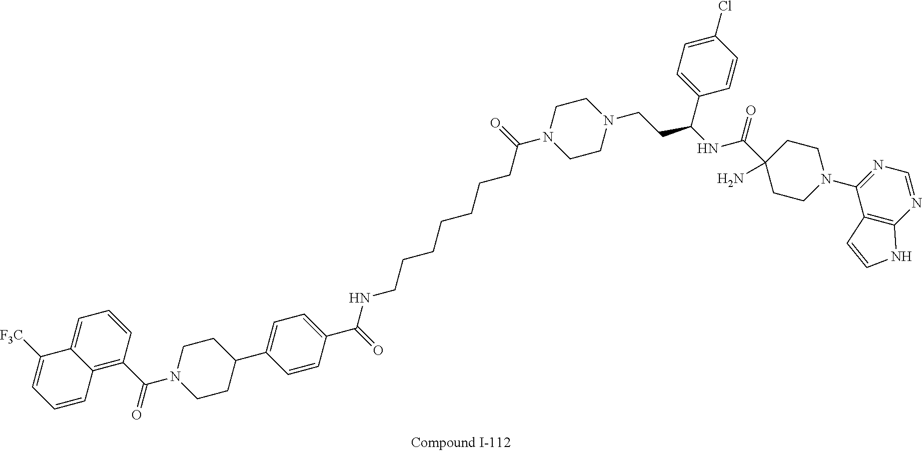 [SMILES: NC1(C(=O)N[C@@H](CCN2CCN(C(=O)CCCCCCCNC(=O)c3ccc(C4CCN(C(=O)c5cccc6c(C(F)(F)F)cccc56)CC4)cc3)CC2)c2ccc(Cl)cc2)CCN(c2ncnc3[nH]ccc23)CC1]

### Step 1: Synthesis of I-112-2

To a solution of I-112-1 (80 mg, 137.30 μmol, 1 eq) in H₂O (1 mL), MeOH (1 mL) and THF (1 mL) was added LiOH·H₂O (17.29 mg, 411.91 μmol, 3 eq). The mixture was stirred at 25° C. for 1 hr. The reaction was concentrated under reduced pressure to give a residue. Then water phase was washed with saturated aqueous citric acid adjust pH=5, extracted with EA 12 mL (4 mL*3). Then dried over Na₂SO₄, filtered and concentrated under reduced pressure to give I-112-2 (80 mg, crude) as a white solid. LCMS (Method E): R^{t}=0.571 min, [M+H]⁺=569.4.

### Step 2: Synthesis of I-112-4

To a solution of I-112-2 (80 mg, 140.69 μmol, 1 eq) and I-112-3 (92.41 mg, 154.76 μmol, 1.1 eq) in DCM (2 mL) was added EDCI (80.91 mg, 422.07 μmol, 3 eq), NMM (142.31 mg, 1.41 mmol, 154.68 μL, 10 eq) and HOAt (28.72 mg, 211.04 μmol, 29.52 μL, 1.5 eq). The mixture was stirred at 25° C. for 1 hr. The reaction was cooled to room temperature, which was diluted with H₂O (5 mL), extracted with DCM 30 mL (10 mL*3), the organic phase was washed with saturated aqueous NaCl (5 mL). Then dried over [Na₂SO₄], filtered and concentrated under reduced pressure to give I-112-4 (200 mg, crude) as a yellow solid, and it was used into the next step without further purification. LCMS (Method G): R^{t}=0.769 min, [M+H]⁺=1147.7.

### Step 3: Synthesis of I-112

The mixture of I-112-4 (200 mg, 174.25 μmol, 1 eq) in DCM (2 mL) and HCl/dioxane (0.5 mL) was stirred at 25° C. for 0.5 hr. The reaction was filtered and concentrated under reduced pressure to give a residue. The residue was purified by prep-HPLC (column: Waters Xbridge 150*25 mm*5 um; mobile phase: [water (NH₄HCO₃)-ACN]; gradient: 48%-78% B over 9 min). The eluent was concentrated under reduced pressure to remove ACN, then lyophilized to afford I-112 (129 mg, 119.25 μmol, 68.44% yield, 96.850% purity) as a white solid. LCMS (Method G): R^{t}=0.726 min, [M+H]⁺=1047.6. SFC: R^{t}=0.879 min. ¹H NMR (400 MHZ, METHANOL-d4) δ=8.42-7.88 (m, 4H), 7.86-7.54 (m, 5H), 7.43-7.22 (m, 6H), 7.11 (d, J=3.6 Hz, 1H), 6.61 (d, J=3.6 Hz, 1H), 5.08-4.91 (m, 2H), 4.55-4.41 (m, 2H), 3.68-3.46 (m, 6H), 3.45-3.33 (m, 3H), 3.28-3.00 (m, 2H), 2.99-2.87 (m, 1H), 2.52-2.28 (m, 8H), 2.25-2.00 (m, 4H), 1.99-1.81 (m, 2H), 1.75-1.54 (m, 7H), 1.53-1.25 (m, 7H).

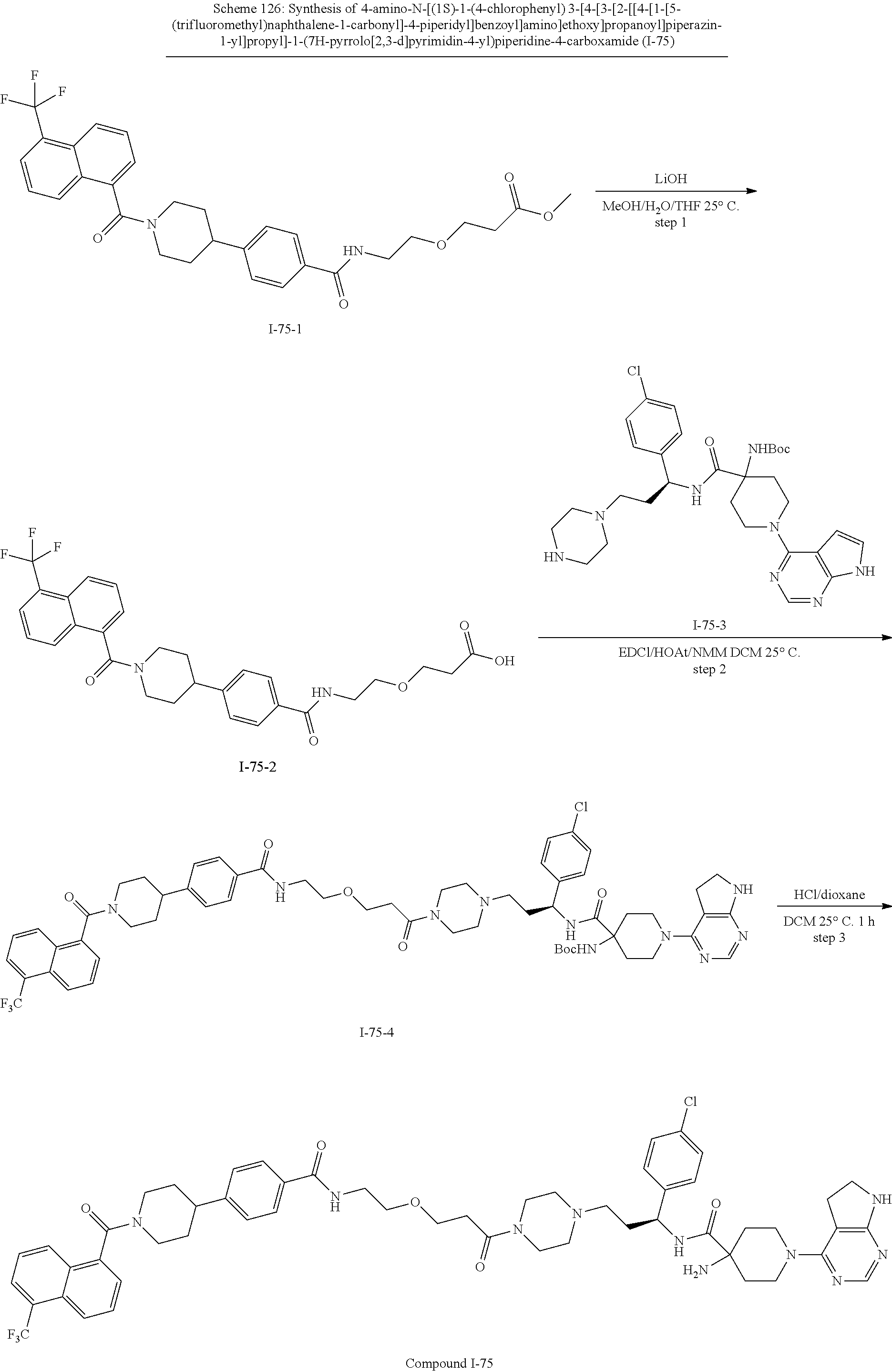

### Step 1: Synthesis of I-75-2

To a solution of I-75-1 (100 mg, 179.67 μmol, 1 eq) in H₂O (1 mL), MeOH (1 mL) and THF (1 mL) was added LiOH·H₂O (22.62 mg, 539.01 μmol, 3 eq). The mixture was stirred at 25° C. for 1 hr. The reaction was concentrated under reduced pressure to give a residue. Then water phase was washed with saturated aqueous citric acid adjust pH=5, extracted with EA 12 mL (4 mL*3). Then dried over Na₂SO₄, filtered and concentrated under reduced pressure to give I-75-2 (100 mg, crude) as a white solid, and it was used into the next step without further purification. LCMS: R^{t}=0.534 min, [M+H]⁺=543.4.

### Step 2: Synthesis of I-75-4

To a solution of I-75-2 (100 mg, 184.32 μmol, 1 eq) and I-75-3 (110.06 mg, 184.32 μmol, 1 eq) in DCM (2 mL) was added EDCI (106.00 mg, 552.95 μmol, 3 eq), NMM (186.43 mg, 1.84 mmol, 202.64 UL, 10 eq) and HOAt (37.63 mg, 276.47 μmol, 38.68 μL, 1.5 eq). The mixture was stirred at 25° C. for 0.5 hr. The reaction was cooled to room temperature, which was diluted with H₂O (5 mL), extracted with DCM 30 mL (10 mL*3), the organic phase was washed with saturated aqueous NaCl (5 mL). Then dried over [Na₂SO₄], filtered and concentrated under reduced pressure to give I-75-4 (200 mg, 178.30 μmol, 96.74% yield) as a yellow solid, and it was used into the next step without further purification. LCMS (Method G): R^{t}=0.695 min, [M+H]⁺=1122.6.

### Step 3: Synthesis of I-75

The mixture of I-75-4 (200 mg, 178.30 μmol, 1 eq) in DCM (2 mL) and HCl/dioxane (0.5 mL) was stirred at 25° C. for 1 hr. The reaction was filtered and concentrated under reduced pressure to give a residue. The residue was purified by prep-HPLC (column: Waters Xbridge 150*25 mm*5 um; mobile phase: [water (NH₄HCO₃)-ACN]; gradient: 38%-68% B over 9 min). The eluent was concentrated under reduced pressure to remove ACN, then lyophilized to afford I-75 (84 mg, 81.00 μmol, 45.43% yield, 98.511% purity) as a white solid. LCMS (Method G): R^{t}=0.639 min, [M+H]⁺=1021.6. SFC: R^{t}=0.756 min. 1H NMR (400 MHZ, METHANOL-d4) δ=8.29-7.98 (m, 4H), 7.84-7.56 (m, 5H), 7.40-7.24 (m, 6H), 7.10 (d, J=3.6 Hz, 1H), 6.60 (d, J=3.6 Hz, 1H), 5.05-4.90 (m, 2H), 4.54-4.42 (m, 2H), 3.82-3.72 (m, 2H), 3.67-3.52 (m, 10H), 3.45-3.36 (m, 1H), 3.22-3.02 (m, 2H), 2.98-2.87 (m, 1H), 2.64-2.62 (m, 2H), 2.47-2.38 (m, 1H), 2.37-2.12 (m, 7H), 2.06-1.79 (m, 4H), 1.72-1.44 (m, 4H).

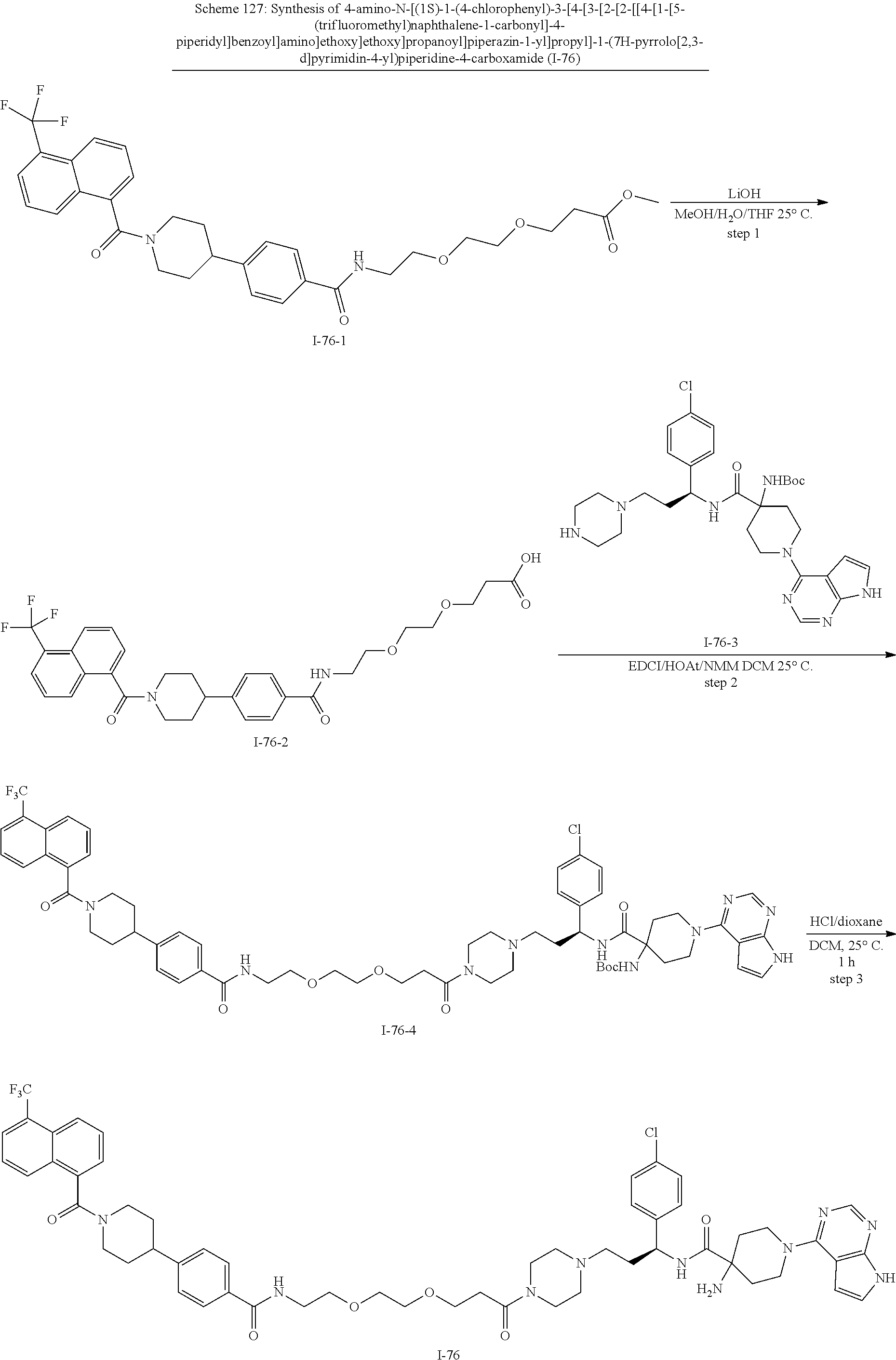

### Step 1: Synthesis of I-76-2

To a solution of I-76-1 (100 mg, 166.49 μmol, 1 eq) in THF (1 mL), MeOH (1 mL) and H₂O (1 mL) was added LiOH·H₂O (6.99 mg, 166.49 μmol, 1 eq). The mixture was stirred at 25° C. for 1 hr. The reaction was concentrated under reduced pressure to give a residue. Then water phase was washed with saturated aqueous citric acid adjust pH=5, extracted with EA 12 mL (4 mL*3). Then dried over Na₂SO₄, filtered and concentrated under reduced pressure to give I-76-2 (100 mg, crude) as a white solid, and it was used into the next step without further purification. LCMS: R^{t}=0.536 min, [M+H]⁺=587.4.

### Step 2: Synthesis of I-76-4

To a solution of I-76-2 (100 mg, 170.47 μmol, 1 eq) and I-76-3 (101.80 mg, 170.47 μmol, 1 eq) in DCM (2 mL) was added EDCI (98.04 mg, 511.42 μmol, 3 eq), NMM (172.43 mg, 1.70 mmol, 187.42 μL, 10 eq) and HOAt (34.81 mg, 255.71 μmol, 35.77 μL, 1.5 eq). The mixture was stirred at 25° C. for 0.5 hr.

The reaction was cooled to room temperature, which was diluted with H₂O (5 mL), extracted with DCM 30 mL (10 mL*3), the organic phase was washed with saturated aqueous NaCl (5 mL). Then dried over [Na₂SO₄], filtered and concentrated under reduced pressure to give I-76-4 (200 mg, crude) as a white solid, and it was used into the next step without further purification. LCMS (Method G): R^{t}=0.693 min, [M+H]⁺=1165.6.

### Step 3: Synthesis of I-76

The mixture of I-76-4 (200 mg, 171.57 μmol, 1 eq) in DCM (2 mL) and HCl/dioxane (0.5 mL) was stirred at 25° C. for 1 hr. The reaction was filtered and concentrated under reduced pressure to give a residue. The residue was purified by prep-HPLC (column: Waters Xbridge 150*25 mm*5 um; mobile phase: [water (NH₄HCO₃)-ACN]; gradient: 38%-68% B over 9 min). The eluent was concentrated under reduced pressure to remove ACN, then lyophilized to afford I-76 (113 mg, 104.56 μmol, 60.94% yield, 98.599% purity) as yellow oil. LCMS (Method G): R^{t}=0.645 min, [M+H]⁺=1067.6. SFC: R^{t}=0.754 min. ¹H NMR (400 MHZ, METHANOL-d4) δ=8.28-7.99 (m, 4H), 7.81-7.56 (m, 5H), 7.39-7.26 (m, 6H), 7.10 (d, J=3.6 Hz, 1H), 6.59 (d, J=3.6 Hz, 1H), 5.04-4.94 (m, 2H), 4.55-4.42 (m, 2H), 3.75-3.73 (m, 2H), 3.67-3.52 (m, 14H), 3.45-3.39 (m, 1H), 3.26-3.01 (m, 2H), 2.99-2.86 (m, 1H), 2.60-2.57 (m, 2H), 2.48-2.27 (m, 6H), 2.23-2.09 (m, 2H), 2.07-1.76 (m, 4H), 1.74-1.40 (m, 4H).

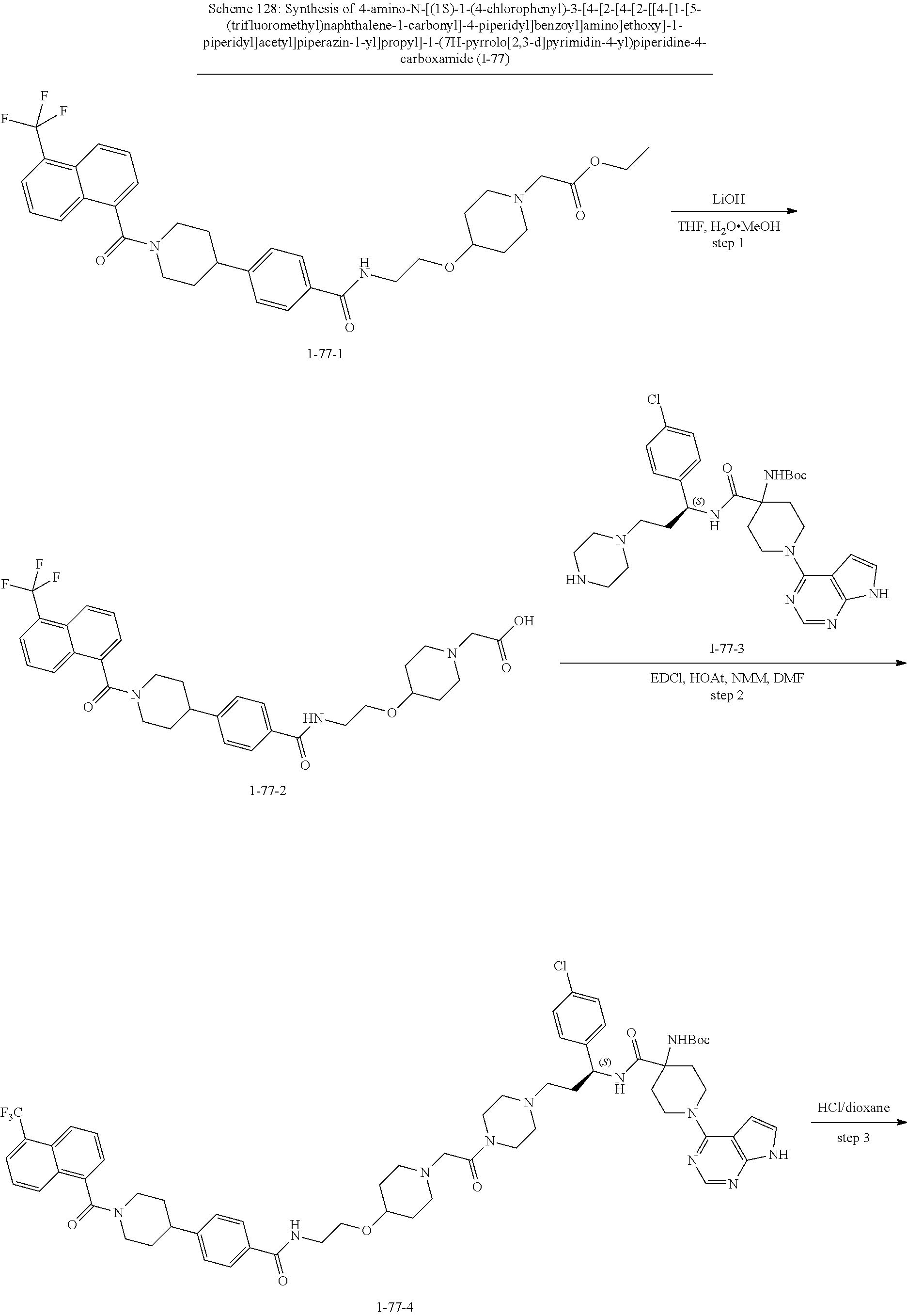

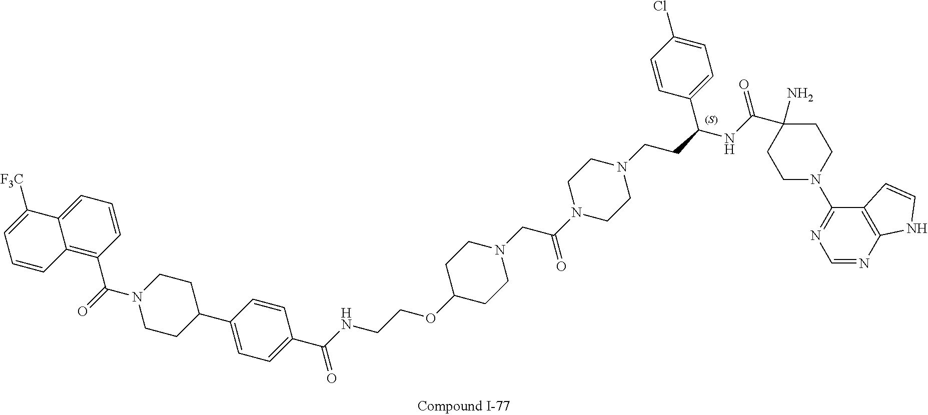

### Step 1: Synthesis of I-77-2

To a solution of I-77-1 (100 mg, 156.32 μmol, 1 eq) in THF (1 mL), H₂O (0.5 mL) and EtOH (0.5 mL) was added LiOH (11.23 mg, 468.97 μmol, 3 eq). The mixture was stirred at 25° C. for 1 hr. The mixture was poured into water 10 mL, then HCl (0.5 mol) was added to make pH<7 and extracted with EA (10 mL*3). The combined organic layers were concentrated to give I-77-2 (90 mg, 136.87 μmol, 87.56% yield, 93.02% purity) as a brown solid. LCMS (Method E): R^{t}=0.565 min, [M+H]⁺=612.3.

### Step 2: Synthesis of I-77-4

To a solution of I-77-2 (80 mg, 130.79 μmol, 1 eq) and I-77-3 (85.91 mg, 143.87 μmol, 1.1 eq) in DMF (1 mL) was added EDCI (75.22 mg, 392.38 μmol, 3 eq), HOAt (17.80 mg, 130.79 μmol, 18.30 μL, 1 eq) and NMM (132.29 mg, 1.31 mmol, 143.80 μL, 10 eq). The mixture was stirred at 25° C. for 1 hr. The mixture was poured into water 10 mL and extracted with EA (10 mL*3). The combined organic layers were concentrated to give a residue. The product was purified by prep-HPLC (column: YMC-Actus Triart C18 150*30 mm*7 um; mobile phase: [water (FA)-ACN]; gradient: 20%-50% B over 10 min) and the eluent was concentrated to remove MeCN and then lyophilization. I-77-4 (90 mg, 75.58 μmol, 57.79% yield, 100% purity) was obtained as a brown solid. LCMS (Method E): R^{t}=0.521 min, [M+H]⁺=1191.7.

### Step 3: Synthesis of 1-77

To a solution of I-77-4 (80 mg, 67.18 μmol, 1 eq) in dioxane (1 mL) was added HCl/dioxane (4 M, 1.33 mL, 79.39 eq). The mixture was stirred at 25° C. for 0.5 hr. The reaction mixture was concentrated under reduced pressure, then was added H₂O to lyophilization to give I-77 (67.24 mg, 59.66 μmol, 88.80% yield, 100% purity, HCl) as a white solid. LCMS (Method E): R^{t}=0.460 min, [M+H]⁺=1090.7. SFC: R^{t}=1.536 min, ee value=100%. ¹H NMR (400 MHZ, METHANOL-d4) δ=8.39 (s, 1H), 8.29-7.99 (m, 3H), 7.83-7.57 (m, 5H), 7.46-7.33 (m, 7H), 7.03-6.98 (m, 1H), 5.09-4.95 (m, 2H), 4.61 (d, J=2.8 Hz, 3H), 4.49-4.36 (m, 1H), 4.34-4.20 (m, 1H), 4.00-3.88 (m, 3H), 3.82-3.75 (m, 1H), 3.73-3.65 (m, 4H), 3.62-3.53 (m, 4H), 3.47-3.36 (m, 4H), 3.28-3.05 (m, 6H), 3.02-2.90 (m, 1H), 2.89-2.79 (m, 1H), 2.78-2.68 (m, 1H), 2.60-2.48 (m, 1H), 2.38-2.30 (m, 1H), 2.24 (d, J=14.4 Hz, 2H), 2.17-1.97 (m, 5H), 1.96-1.79 (m, 2H), 1.75-1.68 (m, 1H), 1.53-1.24 (m, 1H), ¹⁹F NMR (377 MHz, METHANOL-d4) δ=−60.977.

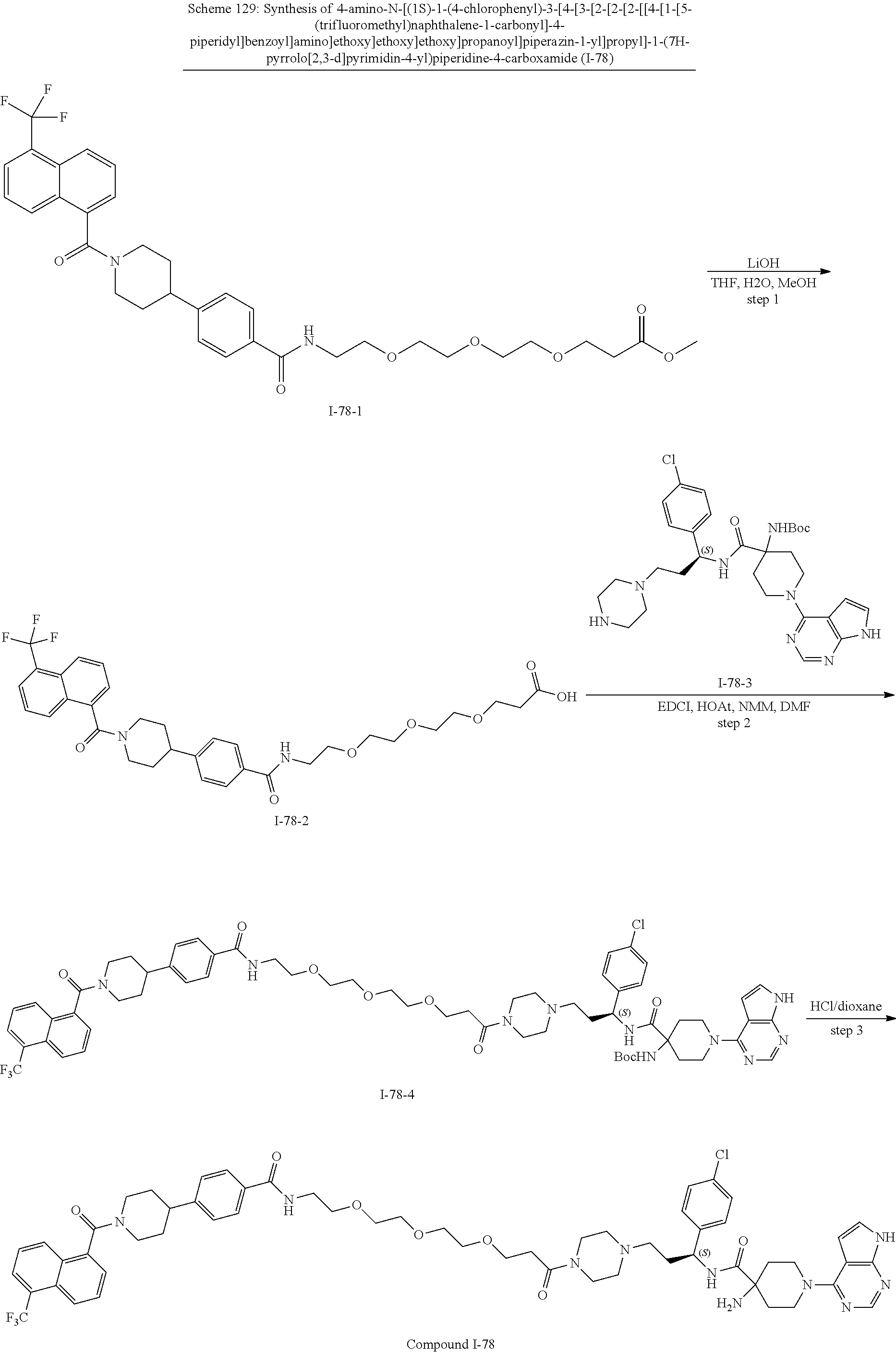

### Step 1: Synthesis of I-78-2

To a solution of I-78-1 (100 mg, 155.12 μmol, 1 eq) in THF (1 mL), H₂O (0.5 mL) and MeOH (0.5 mL) was added LiOH (11.14 mg, 465.35 μmol, 3 eq). The mixture was stirred at 25° C. for 1 hr. The mixture was poured into water 10 mL and extracted with EA (10 mL*3). The combined organic layers were concentrated to give I-78-2 (96 mg, 147.70 μmol, 95.22% yield, 97.03% purity) as a brown solid. LCMS (Method E): R^{t}=0.524 min, [M+H]⁺=631.3. ¹H NMR (400 MHZ, METHANOL-d4) δ=8.29-7.98 (m, 3H), 7.83-7.56 (m, 5H), 7.38-7.30 (m, 2H), 5.07-4.97 (m, 1H), 3.71-3.55 (m, 14H), 3.49-3.40 (m, 1H), 3.27-3.05 (m, 2H), 3.02-2.90 (m, 1H), 2.55-2.46 (m, 2H), 1.96-1.84 (m, 1H), 1.76-1.68 (m, 1H), 1.29 (s, 2H).

### Step 2: Synthesis of I-78-4

To a solution of I-78-2 (86 mg, 136.37 μmol, 1 eq) and I-78-3 (89.57 mg, 150.00 μmol, 1.1 eq) in DMF (1 mL) was added EDCI (78.43 mg, 409.10 μmol, 3 eq), HOAt (18.56 mg, 136.37 μmol, 19.08μ, 1 eq) and NMM (137.93 mg, 1.36 mmol, 149.93 μL, 10 eq). The mixture was stirred at 25° C. for 1 hr. The mixture was poured into water 10 mL and extracted with EA (10 mL*3). The combined organic layers were concentrated to give a residue. The product was purified by prep-HPLC (column: YMC-Actus Triart C18 150*30 mm*7 um; mobile phase: [water (FA)-ACN]; gradient: 28%-58% B over 10 min) and the eluent was concentrated to remove MeCN and then lyophilization. I-78-4 (80 mg, 66.13 μmol, 48.49% yield, 100% purity) was obtained as a brown solid. LCMS (Method E): R^{t}=0.531 min, [M+H]⁺=1209.7.

### Step 3: Synthesis of I-78

To a solution of I-78-4 (75 mg, 61.99 μmol, 1 eq) in dioxane (1 mL) was added HCl/dioxane (4 M, 1.25 mL, 80.65 eq). The mixture was stirred at 25° C. for 0.5 hr. The reaction mixture was concentrated under reduced pressure, then was added H₂O to lyophilization to give I-78 (61.05 mg, 52.73 μmol, 85.06% yield, 99% purity, HCl) as an off-white solid. LCMS (Method E): R^{t}=0.475 min, [M+H]⁺=1109.7. SFC: R^{t}=1.049 min, ee value=100%. ¹H NMR (400 MHZ, METHANOL-d4) δ=8.40-8.35 (m, 1H), 8.29-7.98 (m, 3H), 7.82-7.57 (m, 5H), 7.46-7.30 (m, 7H), 6.99 (d, J=3.6 Hz, 1H), 5.08-4.96 (m, 2H), 4.61 (d, J=12.8 Hz, 3H), 4.28-4.16 (m, 1H), 4.03-3.88 (m, 2H), 3.49 (d, J=3.6 Hz, 20H), 3.21-3.05 (m, 4H), 3.02-2.92 (m, 2H), 2.90-2.80 (m, 1H), 2.79-2.68 (m, 2H), 2.59-2.45 (m, 2H), 2.37-2.19 (m, 2H), 2.06 (d, J=13.6 Hz, 2H), 1.99-1.80 (m, 1H), 1.72-1.70 (m, 1H), 1.52-1.28 (m, 1H). ¹⁹F NMR (377 MHz, METHANOL-d4) δ=−60.971.

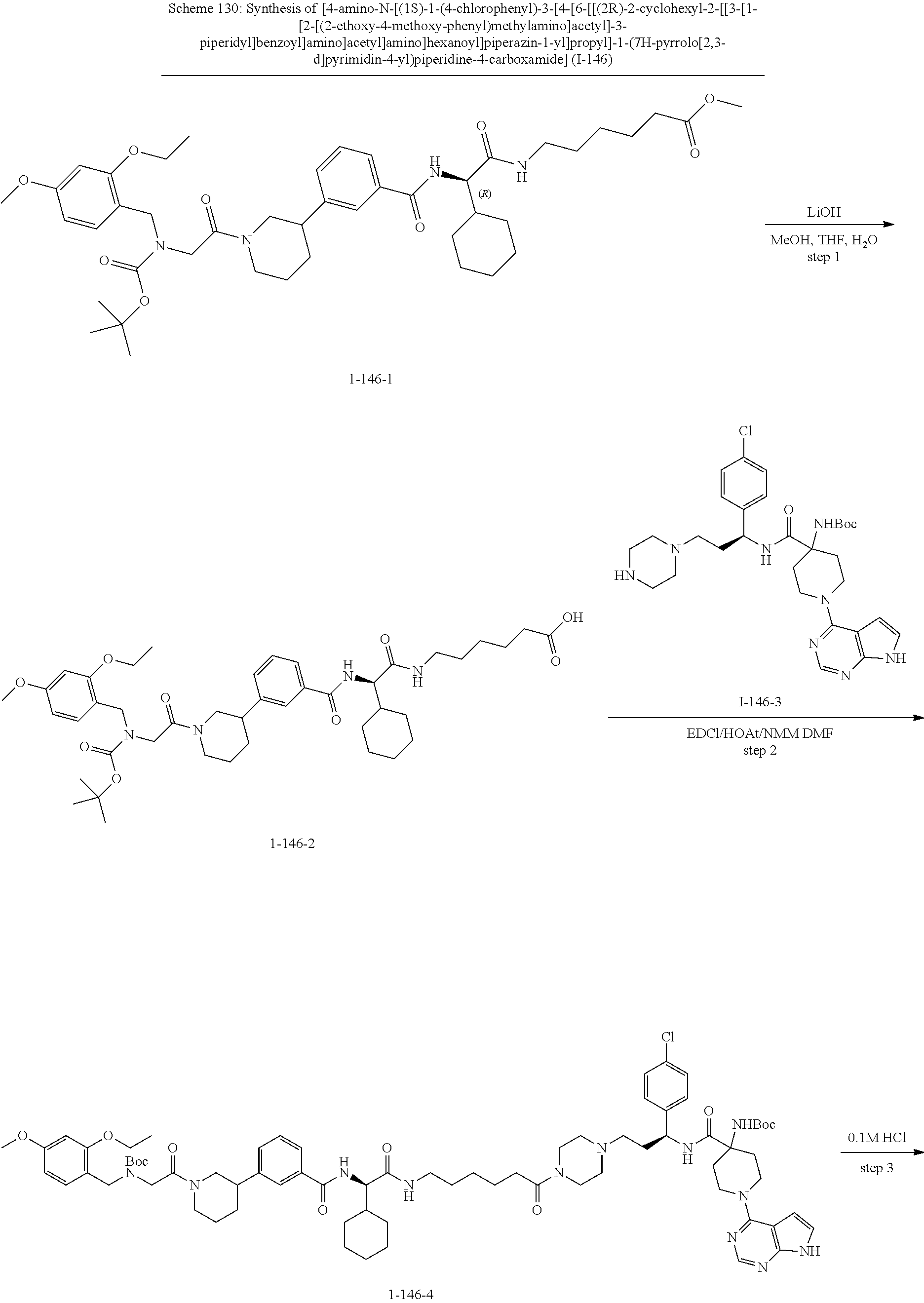

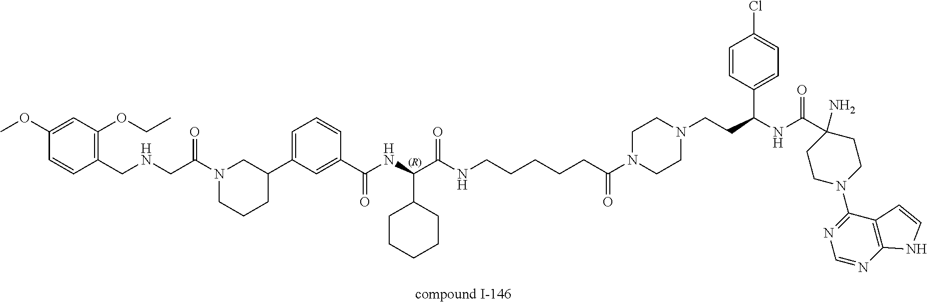

### Step 1: Synthesis of I-146-2

To a solution of I-146-1 (0.1 g, 126.10 μmol, 1 eq) in MeOH (0.4 mL), THF (0.4 mL) and H₂O (0.4 mL) was added LiOH·H₂O (26.46 mg, 630.52 μmol, 5 eq). The mixture was stirred at 25° C. for 1 h. The mixture was added 2 ml H₂O and adjusted to pH 5˜6 with 1M HCl, and mixture was extracted with EA 9 ml (3 ml*3), and combined organic phase was dried with anhydrous sodium sulfate, filtered and filtrate was concentrated to give I-146-2 (90 mg, crude) was obtained as a white solid. LCMS (Method D): R^{t}=0.557 min, [M+H]⁺=779.5. SFC: R^{t}=1.578 min, 2.155 min.

### Step 2: Synthesis of I-146-4

The I-146-2 (90 mg, 115.54 μmol, 1 eq) and I-146-3 (82.79 mg, 138.64 μmol, 1.2 eq) in DMF (1 mL) was added EDCI (66.45 mg, 346.61 μmol, 3 eq), NMM (58.43 mg, 577.68 μmol, 63.51 μL, 5 eq) and HOAt (15.73 mg, 115.54 μmol, 16.16 μL, 1 eq). The mixture was stirred at 25° C. for 1 h. Without workup. The crude product was purified by prep-HPLC (0.1% FA condition) and the eluent was lyophilized to give I-146-4 (130 mg, 95.72 μmol, 82.85% yield, 100% purity) was obtained as a white solid. LCMS (Method D): R^{t}=0.461 min, [M+H]⁺=1357.6.

### Step 3: Synthesis of 1-146

A mixture of I-146-5 (0.12 g, 88.36 μmol, 1 eq) in HCl (0.1 M, 1.20 mL, 1.36 eq), then the mixture was stirred at 100° C. for 1 hr. The mixture was concentrated to give crude product. The residue was purified by prep-HPLC (Fa condition) followed by lyophilization to give I-146 (31.9 mg, 26.61 μmol, 23.70% yield, 96.570% purity) was obtained as a white solid. LCMS (Method D): R^{t}=0.359 min, [M+H]⁺=1157.7 SFC: R^{t}=0.772 min, 0.817 min. ¹H NMR (400 MHZ, METHANOL-d4) δ=8.16 (s, 1H), 7.81-7.68 (m, 2H), 7.50-7.40 (m, 2H), 7.36-7.27 (m, 5H), 7.16 (d, J=3.6 Hz, 1H), 6.66 (d, J=4.0 Hz, 1H), 6.64-6.60 (m, 1H), 6.59-6.55 (m, 1H), 5.02-4.99 (m, 1H), 4.64 (d, J=13.2 Hz, 2H), 4.57-4.43 (m, 1H), 4.38-4.32 (m, 1H), 4.24-4.18 (m, 2H), 4.17-4.10 (m, 2H), 4.06 (d, J=6.8 Hz, 2H), 3.83-3.78 (m, 3H), 3.74-3.65 (m, 1H), 3.63-3.48 (m, 6H), 3.26-3.15 (m, 3H), 3.00-2.72 (m, 2H), 2.53-2.38 (m, 6H), 2.35-2.25 (m, 4H), 2.09-1.97 (m, 3H), 1.90-1.76 (m, 8H), 1.72-1.53 (m, 7H), 1.58-1.43 (m, 3H), 1.40-1.34 (m, 2H), 1.32-1.19 (m, 3H), 1.17-1.04 (m, 2H).

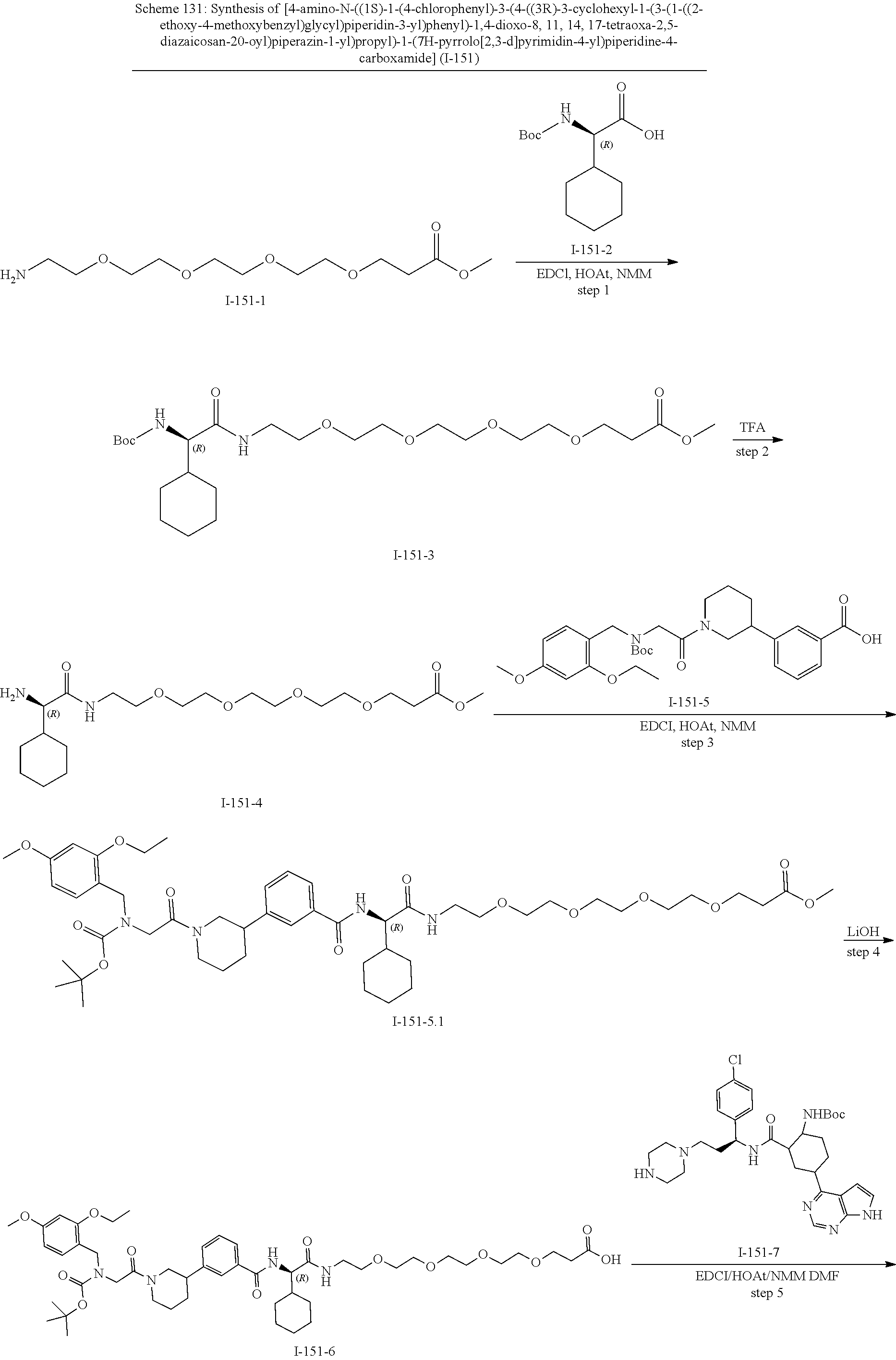

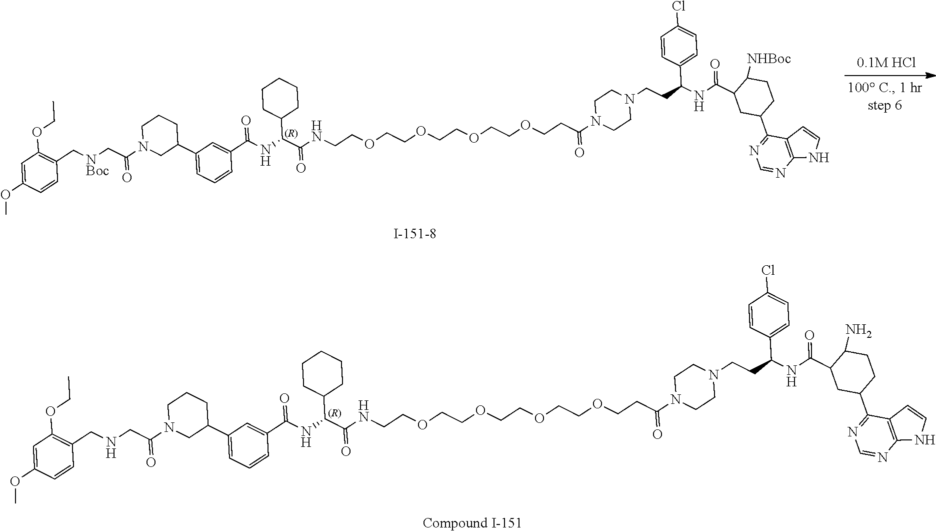

### Step 1: Synthesis of I-151-3.

To a solution of I-151-2 (2 g, 7.77 mmol, 1 eq) in DMF (20 mL) was added HOAt (1.06 g, 7.77 mmol, 1.09 mL, 1 eq) and EDCI (4.47 g, 23.32 mmol, 3 eq), NMM (3.93 g, 38.86 mmol, 4.27 mL, 5 eq) and I-151-1 (2.17 g, 7.77 mmol, 1 eq). The mixture was stirred at 25° C. for 1 hr. The reaction mixture was diluted with H₂O (10 mL) and extracted with EA 30 mL (10 mL*3). The combined organic layers were washed with brine 30 mL (10 mL*3), dried over Na₂SO₄, filtered and concentrated under reduced pressure to give a residue. The residue was used into next step directly. I-151-3 (3 g, crude) was obtained as a yellow oil. LCMS (Method D): R^{t}=0.434 min, [M+H]⁺=519.2.

### Step 2: Synthesis of I-151-4.

To a solution of I-151-3 (3 g, 5.78 mmol, 1 eq) in DCM (30 mL) was added TFA (5 mL). The mixture was stirred at 25° C. for 1 hr. The reaction mixture was concentrated under reduced pressure and washed with PE 30 mL (10 mL*3) to give I-151-4 (3 g, crude, TFA) as a brown oil. LCMS (Method D): R^{t}=0.241 min, [M+H]⁺=419.3.

### Step 3: Synthesis of I-151-5.1.

To a solution of I-151-5 (1 g, 1.90 mmol, 1 eq) in DMF (10 mL) was added HOAt (258.46 mg, 1.90 mmol, 265.63 μL, 1 eq), EDCI (1.09 g, 5.70 mmol, 3 eq), NMM (960.34 mg, 9.49 mmol, 1.04 mL, 5 eq) and I-151-4 (1.11 g, 2.09 mmol, 1.1 eq, TFA). The mixture was stirred at 25° C. for 1 hr. Upon completion, the reaction mixture was diluted with H₂O 3 mL and extracted with EA 15 mL (5 mL*3). The combined organic layers were washed with brine 15 mL (5 mL*3), dried over Na₂SO₄, filtered and concentrated under reduced pressure to give a residue. The residue was purified by flash silica gel chromatography (ISCO®; 40 g SepaFlash® Silica Flash Column, Eluent of 0˜10% MeOH/DCM gradient @ 20 mL/min), the eluent was concentrated under reduced pressure to give I-151-5.1 (1 g, 1.03 mmol, 54.34% yield, 95.667% purity) as a colorless oil. LCMS (Method D): R^{t}=0.498 min, [M+H]⁺=927.4. SFC:
R^{t}=1.595 min, 2.066 min. ¹H NMR (400 MHZ, CHLOROFORM-d) δ=7.74-7.61 (m, 2H), 7.43-7.29 (m, 2H), 7.19-7.08 (m, 1H), 6.95-6.85 (m, 1H), 6.74-6.64 (m, 1H), 6.48-6.37 (m, 2H), 4.79-4.66 (m, 1H), 4.55-4.38 (m, 3H), 4.21-4.12 (m, 1H), 4.06-3.95 (m, 3H), 3.82-3.74 (m, 6H), 3.71-3.61 (m, 16H), 3.60-3.57 (m, 2H), 3.53-3.47 (m, 2H), 3.08-2.97 (m, 1H), 2.79-2.54 (m, 4H), 1.89-1.72 (m, 8H), 1.49-1.44 (m, 9H), 1.43-1.36 (m, 3H), 1.27-1.04 (m, 6H).

### Step 4: Synthesis of I-151-6.

To a solution of I-151-5.1 (150 mg, 161.79 μmol, 1 eq) in THF (0.5 mL), MeOH (0.5 mL), H₂O (0.5 mL) was added LiOH·H₂O (13.58 mg, 323.58 μmol, 2 eq). The mixture was stirred at 25° C. for 1 hr. The pH adjusted to 6-7 with saturated citric acid solution, the reaction mixture was diluted with H₂O 5 mL and extracted with EA 30 mL (10 mL*3). The combined organic layers were washed with brine 15 mL (5 mL*5), dried over [Na₂SO₄], filtered and concentrated under reduced pressure to give I-151-6 (150 mg, crude) as a yellow oil. LCMS (Method D): R^{t}=0.470 min, [M+H]⁺=913.4.

### Step 5: Synthesis of I-151-8.

To a solution of I-151-6 (150 mg, 164.28 μmol, 1 eq) in DMF (2 mL) was added HOAt (22.36 mg. 164.28 μmol, 22.98 μL, 1 eq) and EDCI (94.48 mg, 492.83 μmol, 3 eq), then I-151-7 (98.10 mg, 164.28 μmol, 1 eq) and NMM (83.08 mg, 821.38 μmol, 90.30 μL, 5 eq) was added into the system. The mixture was stirred at 25° C. for 1 hr. The reaction mixture was diluted with H₂O 10 mL and filtered to give a residue. The crude product was used into the next step without further purification. I-151-8 (180 mg, 107.15 μmol, 65.23% yield, 88.832% purity) was obtained as a yellow solid. LCMS (Method D): R^{t}=0.463 min, [M+H]⁺=1491.5.

### Step 6: Synthesis of I-151.

To a solution of I-151-8 (80 mg, 53.61 μmol, 1 eq) in HCl (0.1 M, 0.8 mL). The mixture was stirred at 100° C. for 1 hr. Then HCl (0.1 M, 1.5 mL) was added into the system, the mixture was stirred at 100° C. for 1 hr. The pH adjusted to 7-8 with ammonium hydroxiane, the reaction mixture was diluted with 1 mL MeOH. The residue was purified by prep-HPLC (neutral condition), the eluent was concentrated and lyophilized to give I-151 (21.22 mg, 16.17 μmol, 30.17% yield, 98.465% purity) as a white solid. LCMS (Method D): R^{t}=0.364 min, [M+H]⁺=1291.5. R^{t}=1.855 min, 2.146 min. ¹H NMR (400 MHZ, METHANOL-d4) δ=8.14 (s, 1H), 7.79-7.69 (m, 2H), 7.49-7.39 (m, 2H), 7.37-7.30 (m, 4H), 7.19-7.10 (m, 2H), 6.63 (d, J=3.6 Hz, 1H), 6.56-6.44 (m, 2H), 5.03-4.96 (m, 1H), 4.58-4.46 (m, 3H), 4.45-4.39 (m, 1H), 4.13-4.00 (m, 2H), 3.88-3.80 (m, 1H), 3.79-3.78 (m, 3H), 3.76-3.73 (m, 3H), 3.70-3.66 (m, 1H), 3.62 (s, 14H), 3.57-3.55 (m, 2H), 3.49 (d, J=6.8 Hz, 2H), 3.46-3.43 (m, 1H), 3.40-3.35 (m, 1H), 3.17-3.04 (m, 1H), 2.81-2.67 (m, 2H), 2.65-2.60 (m, 2H), 2.49-2.32 (m, 6H), 2.08 (s, 6H), 1.91-1.68 (m, 9H), 1.67-1.49 (m, 4H), 1.48-1.39 (m, 3H), 1.36-1.24 (m, 3H), 1.24-1.08 (m, 3H).

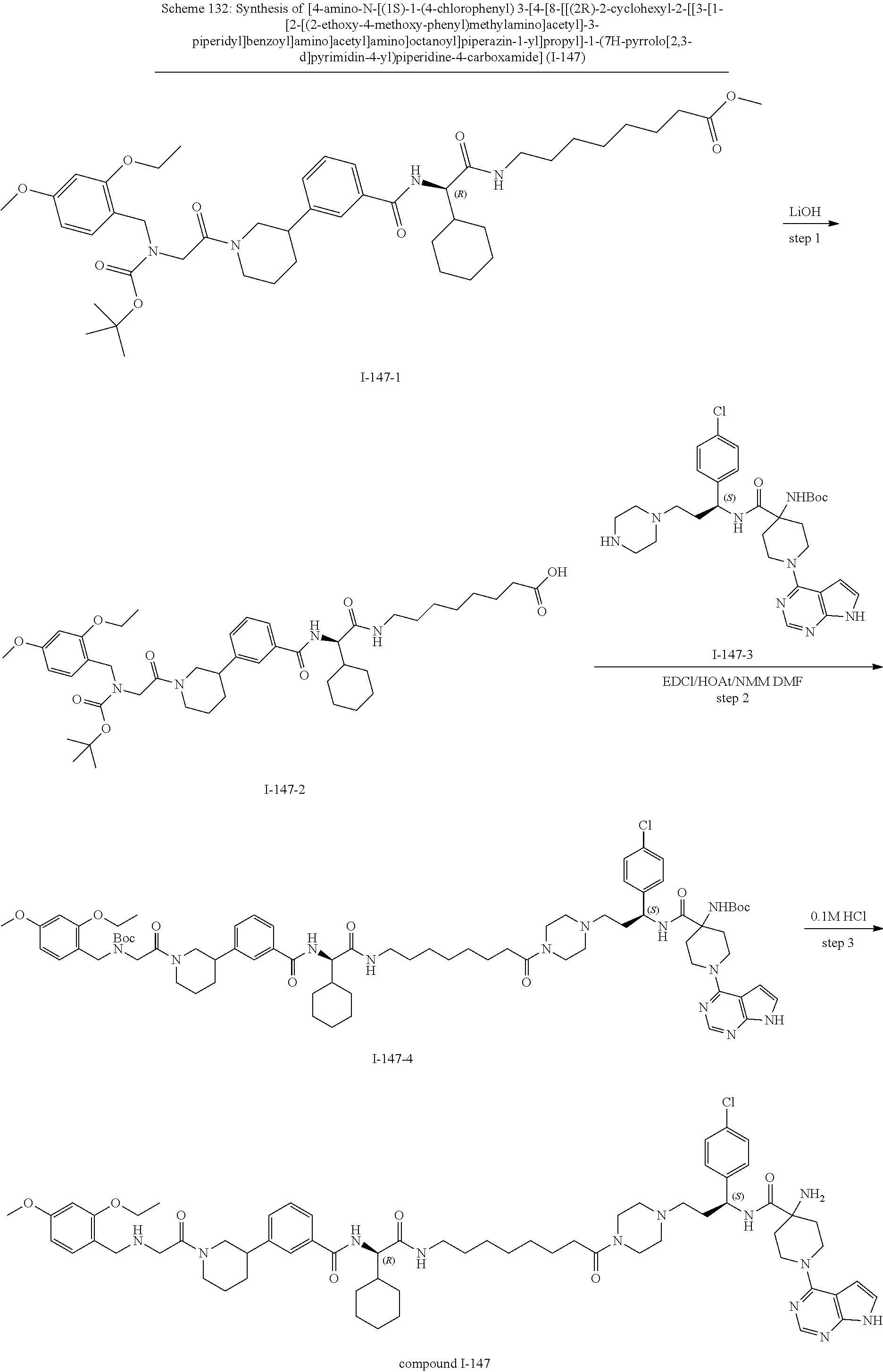

### Step 1: Synthesis of I-147-2

To a solution of I-147-1 (100 mg, 121.80 μmol, 1 eq) in MeOH (0.5 mL), THF (0.5 mL) and H₂O (0.5 mL) was added LiOH·H₂O (25.55 mg, 608.98 μmol, 5 eq). The mixture was stirred at 25° C. for 1 h. The mixture was added 2 ml H₂O and adjusted to pH 5˜6 with 1M HCl, and mixture was extracted with EA 9 ml (3 ml*3), and combined organic phase was dried with anhydrous sodium sulfate, filtered and filtrate was concentrated to give I-147-2 (90 mg, crude) was obtained as a white solid. LCMS (Method D): R^{t}=0.577 min, [M+H]⁺=807.5. SFC: R^{t}=1.985 min, 2.517 min.

### Step 2: Synthesis of I-147-4

The I-147-2 (0.09 g, 111.52 μmol, 1 eq) and I-147-3 (79.91 mg, 133.82 μmol, 1.2 eq) in DMF (1 mL) was added EDCI (64.14 mg, 334.56 μmol, 3 eq), NMM (56.40 mg, 557.60 μmol, 61.30 μL, 5 eq) and HOAt (15.18 mg, 111.52 μmol, 15.60 μL, 1 eq). The mixture was stirred at 25° C. for 1 h. Without workup. The crude product was purified by prep-HPLC (0.1% FA condition) and the eluent was lyophilized to give I-147-4 (130 mg, 93.78 μmol, 84.10% yield, 100% purity) was obtained as a white solid. LCMS (Method D): R^{t}=0.492 min, [M+H]⁺=1386.0.

### Step 3: Synthesis of I-147

A mixture of I-147-5 (0.12 g, 86.57 μmol, 1 eq) in HCl (0.1 M, 1.2 mL, 1.39 eq), then the mixture was stirred at 100° C. for 1 hr. The mixture was concentrated to give crude product. The residue was purified by prep-HPLC (Fa condition) followed by lyophilization to give I-147 (17.21 mg, 14.00 μmol, 13.83% yield, 96.447% purity) was obtained as a white solid. LCMS (Method D): R^{t}=0.366 min, [M+H]⁺=1185.7. SFC: R^{t}=0.779 min, 0.885 min. ¹H NMR (400 MHZ, METHANOL-d4) δ=8.16 (s, 1H), 7.78-7.70 (m, 2H), 7.50-7.42 (m, 2H), 7.34-7.27 (m, 5H), 7.15 (d, J=3.6 Hz, 1H), 6.67-6.60 (m, 2H), 6.59-6.55 (m, 1H), 5.02-4.99 (m, 1H), 4.65-4.59 (m, 2H), 4.55-4.42 (m, 1H), 4.38-4.33 (m, 1H), 4.23-4.11 (m, 4H), 4.06 (d, J=7.2 Hz, 2H), 3.81 (d, J=4.0 Hz, 3H), 3.72-3.65 (m, 1H), 3.62-3.50 (m, 6H), 3.27-3.15 (m, 3H), 3.00-2.72 (m, 2H), 2.51-2.37 (m, 6H), 2.33-2.25 (m, 4H), 2.08-1.98 (m, 3H), 1.92-1.74 (m, 9H), 1.68 (s, 3H), 1.58-1.40 (m, 8H), 1.34-1.22 (m, 7H), 1.17-1.06 (m, 2H)

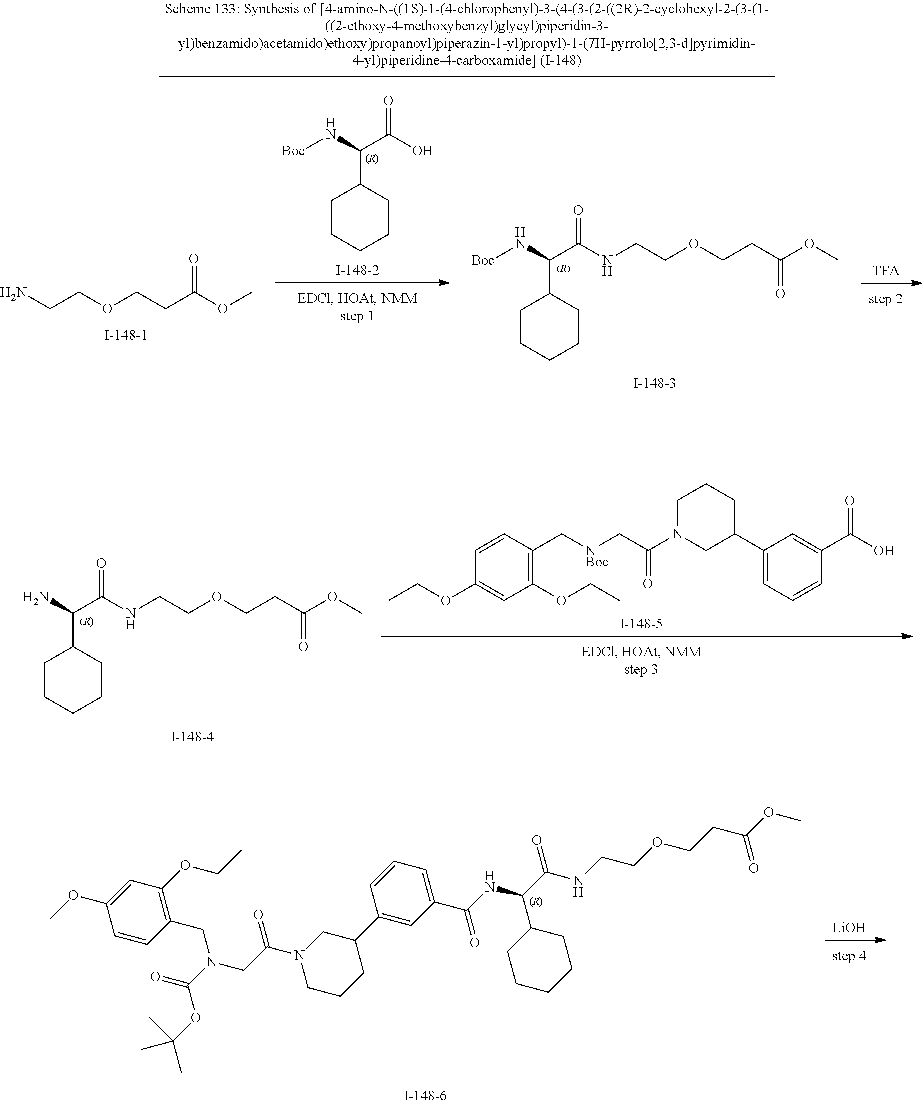

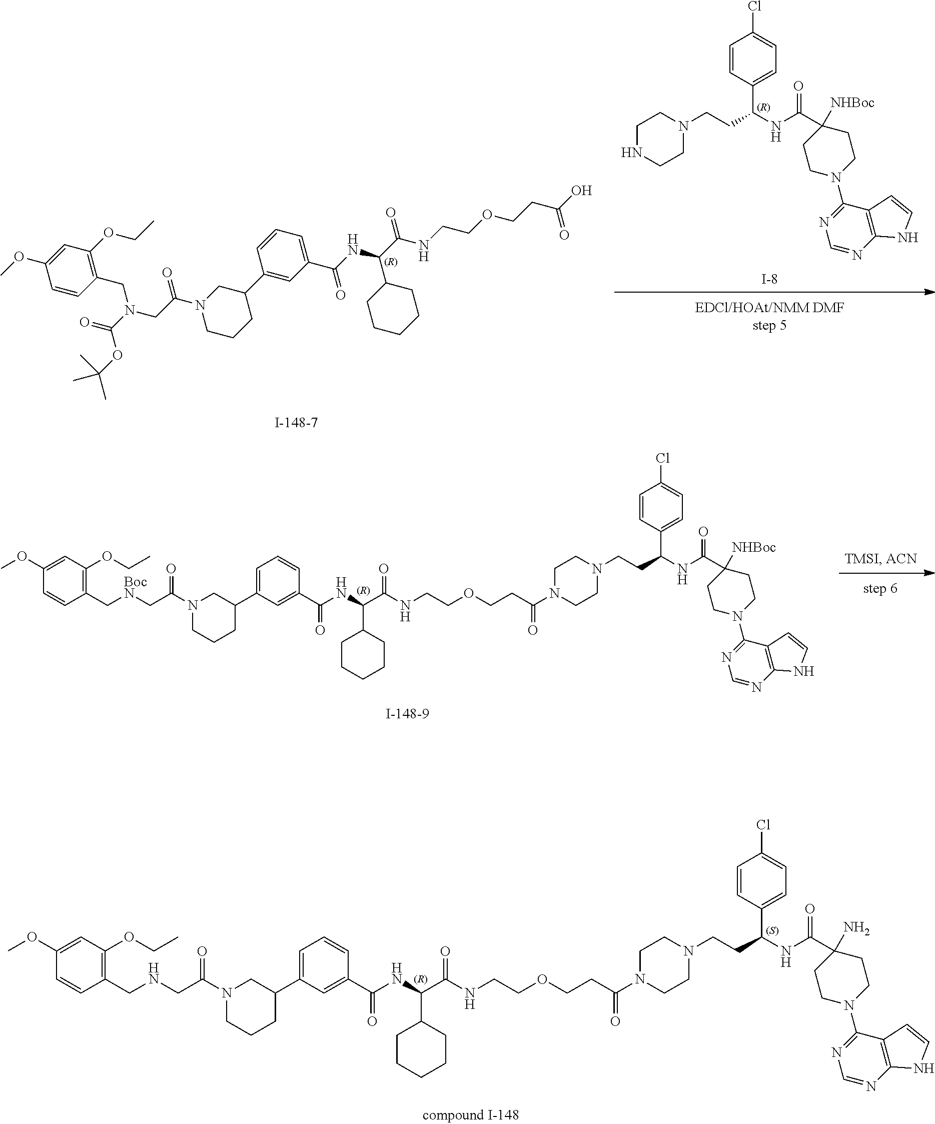

### Step 1: Synthesis of I-148-3

To a solution of I-148-1 (1.72 g. 11.66 mmol, 1 eq). I-148-2 (3 g. 11.66 mmol, 1 eq) in DMF (25 mL) was added EDCI (6.70 g, 34.98 mmol, 3 eq) and HOAt (1.59 g, 11.66 mmol, 1.63 mL, 1 eq), NMM (5.90 g, 58.29 mmol, 6.41 mL, 5 eq). The mixture was stirred at 25° C. for 1 hr. The reaction mixture was diluted with H₂O 50 mL and extracted with EA 300 mL (100 mL*3). The combined organic layers were washed with NaCl 150 mL (50 mL*3), dried over Na₂SO₄, filtered and concentrated under reduced pressure to give a residue. The residue was purified by prep-HPLC (0.1% FA) the eluent was concentrated to remove ACN and lyophilized to get I-148-3 (2 g, 5.17 mmol, 44.39% yield) was obtained as a white solid. LCMS (Method D): R^{t}=0.452 min, [M+H]⁺=387.1. SFC: R^{t}=1.030 min.

### Step 2: Synthesis of I-148-4

To a solution of I-148-3 (1 g, 2.59 mmol, 1 eq) in DCM (10 mL) was added TFA (3.07 g, 26.92 mmol, 2.00 mL, 10.41 eq). The mixture was stirred at 25° C. for 0.5 hr. The reaction mixture was concentrated under reduced pressure to give I-148-4 (1 g, crude, TFA) was obtained as a colorless oil. LCMS (Method D): R^{t}=0.256 min, [M+H]⁺=257.0.

### Step 3: Synthesis of I-148-6

To a solution of I-148-4 (1 g, 2.50 mmol, 1 eq, TFA), I-148-5 (1.32 g, 2.50 mmol, 1 eq) in DMF (10 mL) was added EDCI (1.44 g. 7.49 mmol, 3 eq) and NMM (1.26 g, 12.49 mmol, 1.37 mL, 5 eq), HOAt (339.95 mg, 2.50 mmol, 349.38 μL, 1 eq). The mixture was stirred at 25° C. for 1 hr. The reaction mixture was diluted with H₂O 10 mL and extracted with EA 60 mL (20 mL*3). The combined organic layers were washed with NaCl (aq) 30 mL (10 mL*3), dried over Na₂SO₄, filtered and concentrated under reduced pressure to give a residue. The residue was purified by prep-HPLC (column: Welch Ultimate XB CN 150*25*10 um; mobile phase: [EtOH+MeOH (4:1, neutral)]; gradient: 10%-40% B over 16 min) the eluent was concentrated to get I-148-6 (0.2 g, 251.58 μmol, 10.07% yield) was obtained as a colorless oil. SFC showed two peaks (peak 1: R^{t}=1.265 min, 43.951%; peak2: R^{t}=1.362 min, 49.824%). LCMS: R^{t}=0.578 min, [M+H]⁺=795.4. SFC: R^{t}=1.265 min.

### Step 4: Synthesis of I-148-7

To a solution of I-148-6 (100 mg, 125.79 μmol, 1 eq) in THF (1 mL), H2O (1 mL), MeOH (1 mL) was added LiOH·H2O (15.84 mg, 377.37 μmol, 3 eq). The mixture was stirred at 25° C. for 1 hr. The reaction mixture was adjusted pH=6 by HCl (1M) at 25° C., and then extracted with EA 10 mL (5 mL*2). The combined organic layers were washed with aq. NaCl 5 mL (2.5 mL*2, dried over Na₂SO₄, filtered and concentrated under reduced pressure to give I-148-7 (100 mg, crude) was obtained as a colorless oil. LCMS:

R^{t}=0.544 min, (M+H)=781.3. SFC: R^{t}=1.189 min.

### Step 5: Synthesis of I-148-9

To a solution of I-148-7 (100 mg, 128.05 μmol, 1 eq), I-148-8 (76.47 mg, 128.05 μmol, 1 eq) in DMF (1.5 mL) was added EDCI (24.55 mg, 128.05 μmol, 1 eq) and HOAt (17.43 mg, 128.05 μmol, 17.91 μL, 1 eq), NMM (12.95 mg, 128.05 μmol, 14.08 μL, 1 eq). The mixture was stirred at 25° C. for 1 hr. The reaction mixture was diluted with H₂O 10 mL and extracted with EA 15 mL (5 mL*3). The combined organic layers were washed with NaCl (aq) 10 mL (5 mL*2), dried over Na₂SO₄, filtered and concentrated under reduced pressure to give a residue. The residue was purified by prep-HPLC (0.1% FA) the eluent was concentrated to remove ACN and lyophilized to get I-148-9 (110 mg, 80.88 μmol, 63.16% yield) was obtained as a white solid. LCMS (Method D): R^{t}=0.463 min, [M+H]⁺=1359.7. SFC: R^{t}=2.295 min.

### Step 6: Synthesis of I-148

To a solution of I-148-9 (80 mg, 58.82 μmol, 1 eq) in ACN (0.8 mL) was added TMSI (35.31 mg, 176.46 μmol, 24.02 μL, 3 eq). The mixture was stirred at 0° C. for 0.25 hr. The reaction mixture was quenched by addition NaHCO₃(aq) 10 mL at 25° C., and then extracted with CHCl₃/i-PrOH (3/1) 15 mL (5 mL*3). The combined organic layers dried over Na₂SO₄, filtered and concentrated under reduced pressure to give a residue. The residue was purified by prep-HPLC (column: Waters Xbridge 150*25 mm*5 um; mobile phase: [water (NH3H2O)-ACN]; gradient: 37%-67% B over 11 min) the eluent was concentrated to remove ACN and lyophilized to get I-148 (15.1 mg, 12.90 μmol, 21.93% yield, 99.059% purity) was obtained as a white solid. LCMS (Method D): R^{t}=0.353 min, [M+H]⁺=1159.7. SFC: R^{t}=2.154 min. ¹H NMR (400 MHZ, METHANOL-d4) δ=8.11 (s, 1H), 7.77-7.67 (m, 2H), 7.48-7.38 (m, 2H), 7.35-7.27 (m, 4H), 7.17-7.09 (m, 2H), 6.60 (d, J=4.0 Hz, 1H), 6.54-6.42 (m, 2H), 4.97-4.94 (m, 1H), 4.65-4.55 (m, 1H), 4.53-4.38 (m, 4H), 4.09-4.00 (m, 2H), 3.85-3.78 (m, 1H), 3.77-3.73 (m, 4H), 3.72 (s, 3H), 3.67-3.59 (m, 4H), 3.55-3.54 (m, 4H), 3.50-3.42 (m, 2H), 3.41-3.35 (m, 2H), 3.15-3.01 (m, 1H), 2.77-2.63 (m, 2H), 2.63-2.57 (m, 2H), 2.45-2.38 (m, 2H), 2.36-2.30 (m, 2H), 2.23-2.09 (m, 2H), 2.06-1.91 (m, 3H), 1.88-1.67 (m, 8H), 1.63-1.51 (m, 3H), 1.45-1.37 (m, 3H), 1.35-1.03 (m, 6H).

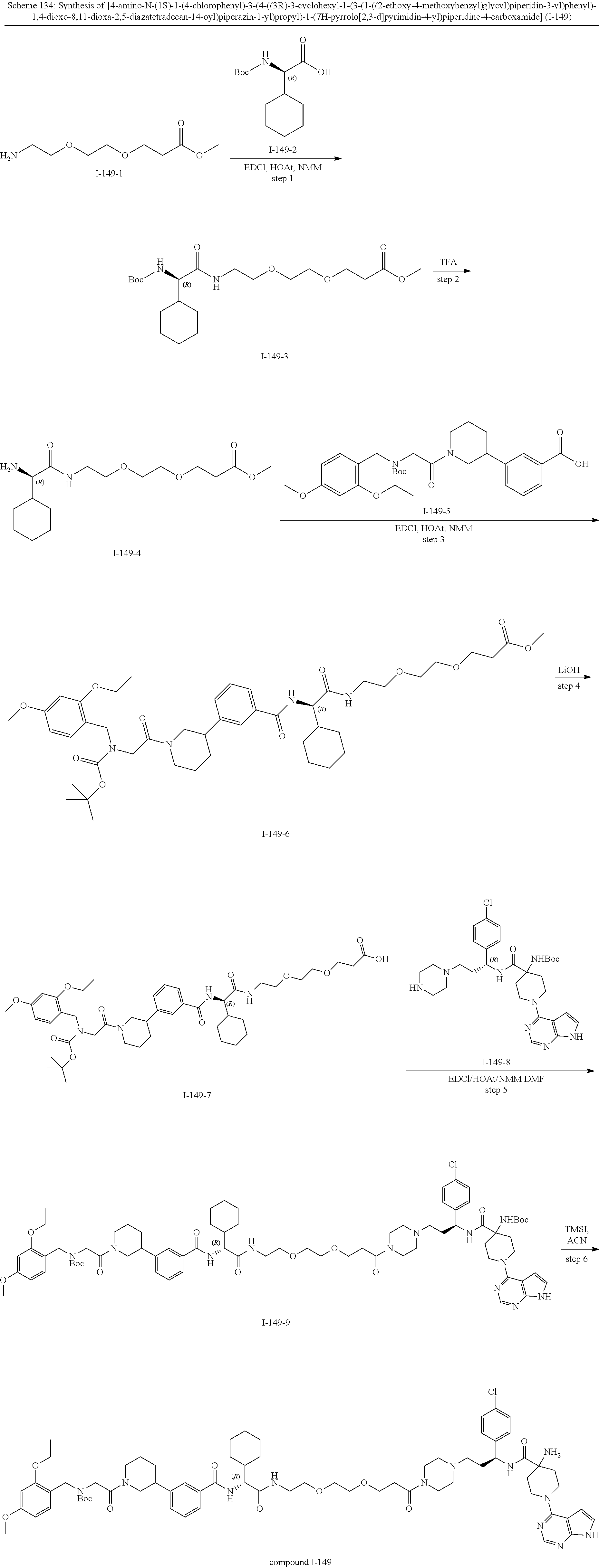

### Step 1: Synthesis of I-149-3

To a solution of I-149-1 (2.23 g, 11.66 mmol, 1 eq), I-149-2 (3 g, 11.66 mmol, 1 eq) in DMF (25 mL) was added EDCI (6.70 g, 34.98 mmol, 3 eq) and HOAt (1.59 g, 11.66 mmol, 1.63 mL, 1 eq), NMM (5.90 g, 58.29 mmol, 6.41 mL, 5 eq). The mixture was stirred at 25° C. for 1 hr. The reaction mixture was diluted with H₂O 50 mL and extracted with EA 300 mL (100 mL*3). The combined organic layers were washed with NaCl 150 mL (50 mL*3), dried over Na₂SO₄, filtered and concentrated under reduced pressure to give a residue. The residue was purified by prep-HPLC (0.1% FA) the eluent was concentrated to remove ACN and lyophilized to get I-149-3 (2 g, 4.65 mmol, 39.85% yield) as a colorless oil. LCMS (Method D): R^{t}=0.451 min, [M+H]⁺=431.2. SFC: R^{t}=1.220 min.

### Step 2: Synthesis of I-149-4

To a solution of I-149-3 (1 g, 2.32 mmol, 1 eq) in DCM (10 mL) was added TFA (3.07 g, 26.92 mmol, 2.00 mL, 11.59 eq). The mixture was stirred at 25° C. for 0.5 hr. The reaction mixture was concentrated under reduced pressure to give I-149-4 (1 g, crude, TFA) as a colorless oil. LCMS (Method D): R^{t}=0.451 min, [M+H]⁺=331.1.

### Step 3: Synthesis of I-149-6

To a solution of I-149-4 (1 g. 2.25 mmol, 1 eq, TFA), I-149-5 (1.18 g. 2.25 mmol, 1 eq) in DMF (10 mL) was added EDCI (1.29 g, 6.75 mmol, 3 eq) and HOAt (306.25 mg, 2.25 mmol, 314.75 μL, 1 eq), NMM (1.14 g, 11.25 mmol, 1.24 mL, 5 eq). The mixture was stirred at 25° C. for 1 hr. The reaction mixture was diluted with H₂O 10 mL and extracted with EA 60 mL (20 mL*3). The combined organic layers were washed with NaCl (aq) 30 mL (10 mL*3), dried over Na₂SO₄, filtered and concentrated under reduced pressure to give a residue. The residue was purified by prep-HPLC (column: Welch Ultimate XB CN 150*25*10 um; mobile phase: [EtOH+MeOH (4:1, neutral)]; gradient: 10%-40% B over 16 min) the eluent was concentrated to get I-149-6 (400 mg, 476.74 μmol, 21.19% yield) as a colorless oil. LCMS: R^{t}=0.585 min, [M+H]⁺=839.3.

### Step 4: Synthesis of I-149-7

To a solution of I-149-6 (100 mg, 119.19 μmol, 1 eq) in THF (1 mL), H2O (1 mL), MeOH (1 mL) was added LiOH·H2O (15.00 mg, 357.56 μmol, 3 eq). The mixture was stirred at 25° C. for 1 hr. The reaction mixture was adjusted pH=6 by HCl (1M) at 25° C., and then extracted with EA 10 mL (5 mL*2). The combined organic layers were washed with NaCl (aq) 5 mL (2.5 mL*2, dried over Na₂SO₄, filtered and concentrated under reduced pressure to give I-149-7 (100 mg, crude) as a colorless oil. LCMS: R^{t}=0.510 min, [M+H]⁺=825.8. SFC: R^{t}=1.486 min.

### Step 5: Synthesis of I-149-9

To a solution of I-149-7 (100 mg, 121.21 μmol, 1 eq), I-149-8 (72.38 mg, 121.21 μmol, 1 eq) in DMF (1.5 mL) was added EDCI (69.71 mg, 363.64 μmol, 3 eq) and HOAt (16.50 mg, 121.21 μmol, 16.96 μL, 1 eq), NMM (61.30 mg, 606.06 μmol, 66.63 μL, 5 eq). The mixture was stirred at 25° C. for 1 hr. The reaction mixture was diluted with H₂O 10 mL and extracted with EA 15 mL (5 mL*3). The combined organic layers were washed with NaCl (aq) 10 mL (5 mL*2), dried over Na₂SO₄, filtered and concentrated under reduced pressure to give a residue. The residue was purified by prep-HPLC (0.1FA %) the eluent was concentrated to remove ACN and lyophilized to get I-149-9 (60 mg, 42.73 μmol, 35.25% yield) as a white solid. LCMS (Method D): R^{t}=0.465 min, [M+H]⁺=1404.3. SFC: R^{t}=2.400 min.

### Step 6: Synthesis of I-149

To a solution of I-149-9 (60 mg, 42.73 μmol, 1 eq) in ACN (0.6 mL) was added TMSI (25.65 mg, 128.19 μmol, 17.45 μL, 3 eq). The mixture was stirred at 0° C. for 0.25 hr. The reaction mixture was quenched by addition NaHCO₃(aq) 10 mL at 25° C., and then extracted with CHCl₃/i-PrOH (3/1) 15 mL (5 mL*3). The combined organic layers dried over Na₂SO₄, filtered and concentrated under reduced pressure to give a residue. The residue was purified by prep-HPLC (column: Waters Xbridge C18 150*50 mm*10 um; mobile phase: [water (NH3H2O)-ACN]; gradient: 38%-68% B over 11 min) the eluent was concentrated to remove ACN and lyophilized to get I-149 (5.66 mg, 4.48 μmol, 10.48% yield, 95.285% purity) as a light yellow solid. LCMS (Method D): R^{t}=0.354 min, [M+H]⁺=1203.7. SFC: R^{t}=0.650 min. ¹H NMR (400 MHZ, METHANOL-d4) δ=8.11 (s, 1H), 7.77-7.67 (m, 2H), 7.46-7.38 (m, 2H), 7.35-7.27 (m, 4H), 7.18-7.07 (m, 2H), 6.60 (d, J=3.6 Hz, 1H), 6.55-6.43 (m, 2H), 4.99-4.96 (m, 1H), 4.63-4.55 (m, 4H), 4.52-4.49 (m, 2H), 4.40-4.38 (m, 1H), 4.09-4.01 (m, 2H), 3.81-3.71 (m, 8H), 3.62-3.61 (m, 4H), 3.58 (s, 4H), 3.55 (s, 1H), 3.51-3.42 (m, 3H), 3.39-3.36 (m, 1H), 3.14-3.04 (m, 1H), 2.78-2.66 (m, 2H), 2.63-2.60 (m, 2H), 2.47-2.41 (m, 2H), 2.38-2.32 (m, 2H), 2.23-2.11 (m, 2H), 2.11-1.91 (m, 4H), 1.86-1.74 (m, 6H), 1.73-1.66 (m, 2H), 1.62-1.49 (m, 3H), 1.46-1.38 (m, 3H), 1.34-1.26 (m, 3H), 1.24-1.10 (m, 3H).

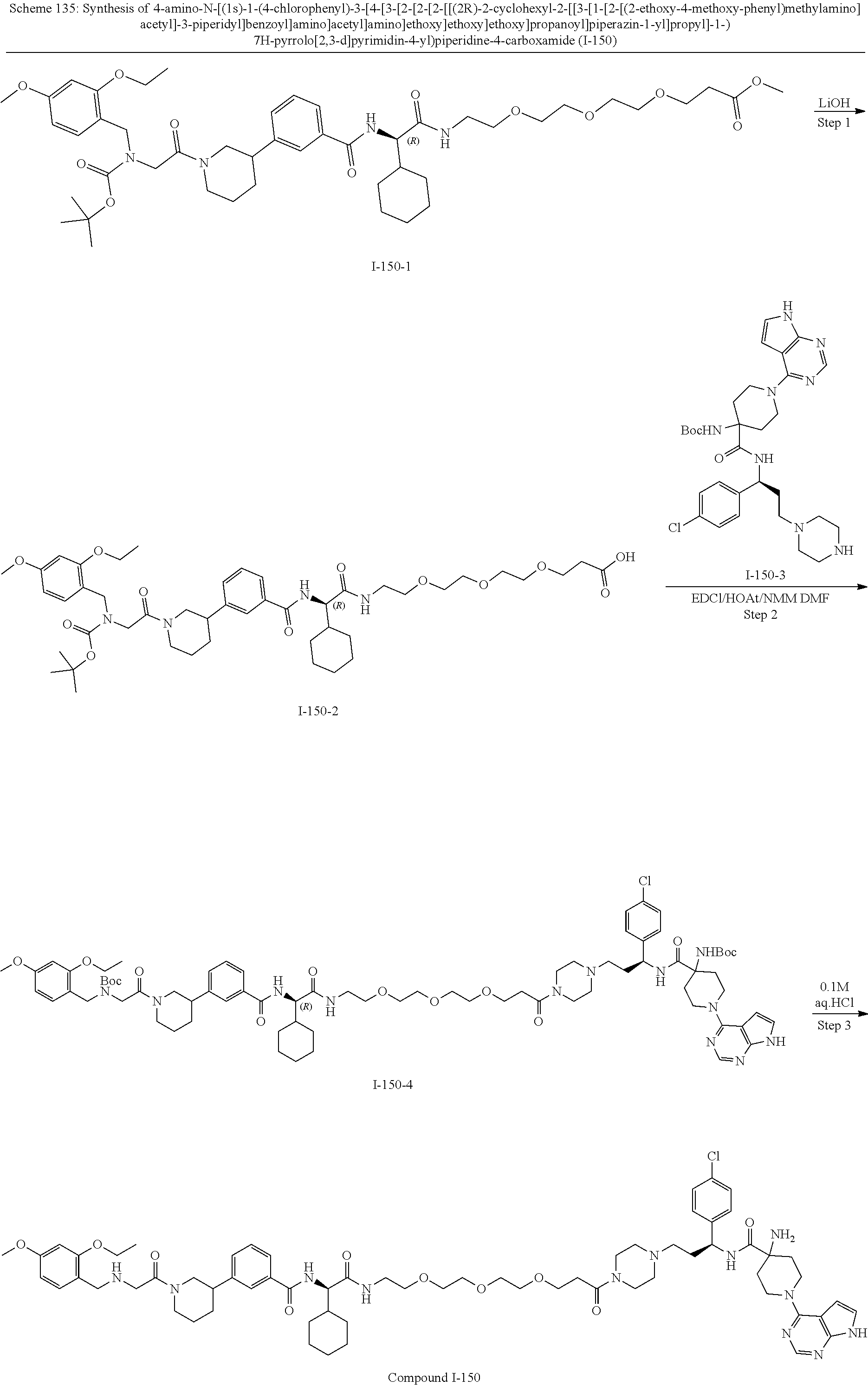

### Step 1: Synthesis of I-150-2

To a solution of I-150-1 (150 mg, 169.86 μmol, 1 eq) in MeOH (0.5 mL) and H₂O (0.5 mL) was added LiOH (12.20 mg, 509.58 μmol, 3 eq) in THF (0.5 mL) were added at 25° C. The mixture was stirred at 25° C. for 1 hr. The pH was adjusted to 7 with 1M HCl, the mixture solution was washed with water (1 mL) and extracted with DCM (1 mL*3), the combined organic phase was dried by Na₂SO₄, concentrated under reduced pressure to give I-150-2 (120 mg, crude) as a white solid. LCMS (Method D): R^{t}=0.474 min, [M+H]⁺=869.4.

### Step 2: Synthesis of I-150-4

To a solution of I-150-2 (120 mg, 138.08 μmol, 1 eq), I-150-3 (82.46 mg, 138.08 μmol, 1 eq) in DMF (1 mL) then the NMM (69.83 mg, 690.41 μmol, 75.91 μL, 5 eq), EDCI (132.35 mg, 690.41 μmol, 5 eq) and HOAt (37.59 mg, 276.16 μmol, 38.63 μL, 2 eq) was added in. The mixture was stirred at 25° C. for 0.5 h. The reaction mixture was washed with water (1 mL) and extracted with EA (1 mL*3), the combined organic phase was dried by Na₂SO₄, concentrated under reduced pressure to give a residue. The residue was purified by flash silica gel chromatography (ISCO®; 4 g SepaFlash® Silica Flash Column, Eluent of 0˜50% Ethyl acetate/Petroleum ether @35 mL/min) and the eluent was concentrated to give product. I-150-4 (130 mg, 89.77 μmol, 65.01% yield, 100% purity) as a white solid. LCMS (Method D): R^{t}=0.467 min, [M+H]⁺=1448.3.

### Step 3: Synthesis of I-150

To a solution of I-150-4 (120 mg, 82.86 μmol, 1 eq) in HCl (0.1 M, 1.20 mL, 1.45 eq). The mixture was stirred at 100° C. for 1 hr. The reaction mixture was filtered to give the filtrate. The residue was purified by reverse-phase (0.1% of FA) and the eluent was lyophilized to give I-150 (58 mg, 44.27 μmol, 53.43% yield, 95.262% purity) as an off-white solid. LCMS (Method D): R^{t}=0.357 min, [M+H]⁺=1247.7. 1H NMR (400 MHZ, METHANOL-d4) δ=8.39 (s, 2H), 8.16 (s, 1H), 7.79-7.69 (m, 2H), 7.49-7.40 (m, 2H), 7.31 (s, 4H), 7.30-7.25 (m, 1H), 7.15 (d, J=3.6 Hz, 1H), 6.64 (d, J=3.6 Hz, 1H), 6.62-6.59 (m, 1H), 6.58-6.53 (m, 1H), 4.99 (t, J=6.8 Hz, 1H), 4.59-4.44 (m, 3H), 4.43-4.39 (m, 1H), 4.25-4.18 (m, 2H), 4.16-4.10 (m, 2H), 4.05 (d, J=8.0 Hz, 1H), 4.02-3.91 (m, 1H), 3.80 (d, J=4.4 Hz, 3H), 3.74-3.64 (m, 5H), 3.61-3.52 (m, 14H), 3.48-3.40 (m, 1H), 3.37-3.32 (m, 1H), 3.23-3.11 (m, 1H), 2.97-2.69 (m, 2H), 2.66-2.43 (m, 9H), 2.37-2.26 (m, 2H), 2.08-1.98 (m, 3H), 1.91-1.73 (m, 9H), 1.72-1.52 (m, 3H), 1.49-1.40 (m, 3H), 1.30-1.22 (m, 2H), 1.21-0.99 (m, 3H).

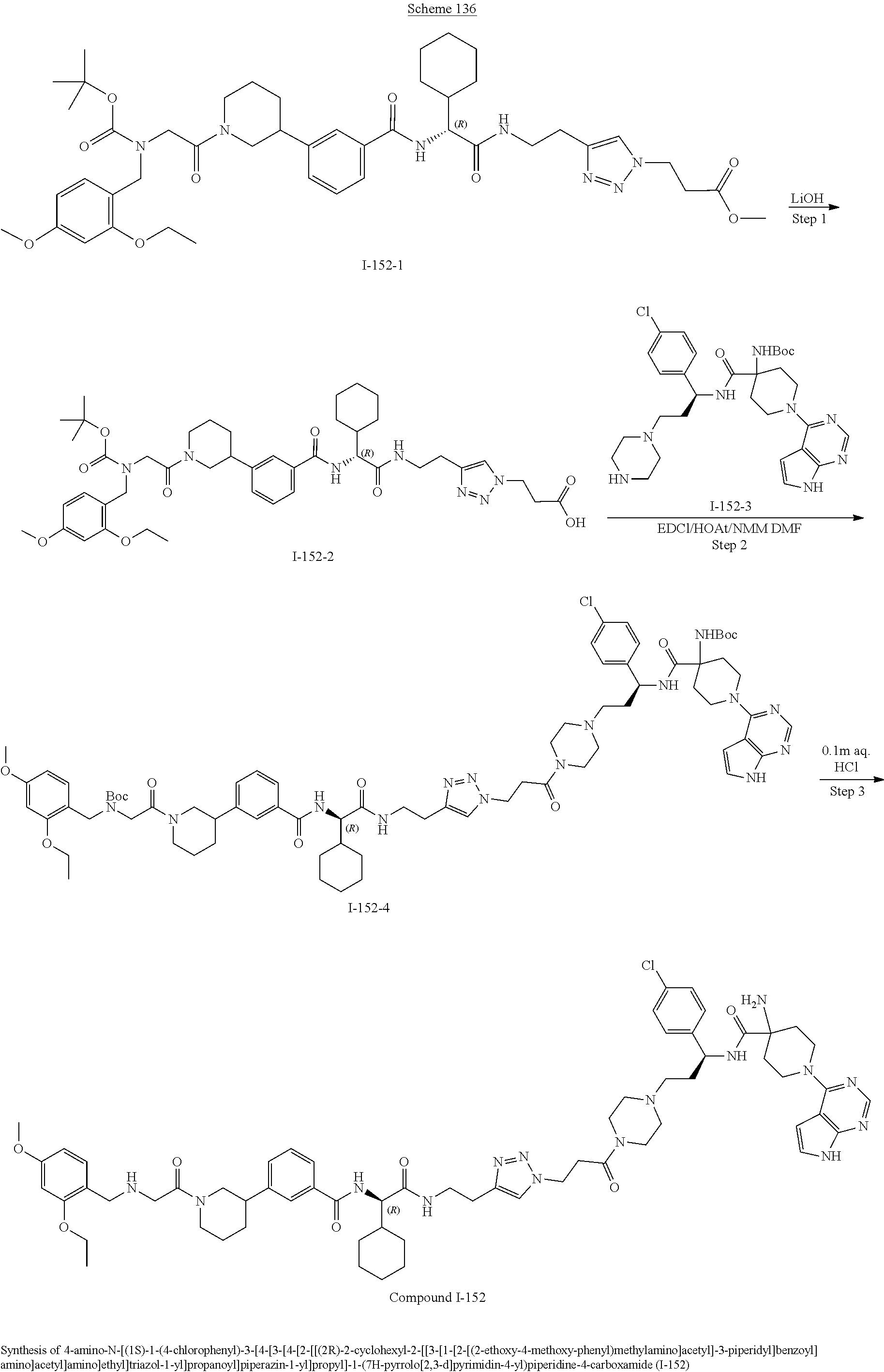

### Step 1: Synthesis of I-152-2

To a solution of I-152-1 (100 mg, 118.20 μmol, 1 eq) in H₂O (0.3 mL) and MeOH (0.3 mL) was added LiOH (8.49 mg, 354.60 μmol, 3 eq) in THF (0.3 mL) were added at 25° C. The mixture was stirred at 25° C. for 1 hr. The pH was adjusted to 7 with 1M HCl, The reaction mixture was washed with water (1 mL) and extracted with DCM (1 mL*3), the combined organic phase was dried by Na₂SO₄, concentrated under reduced pressure to give I-152-2 (120 mg, crude) as a white solid. LCMS (Method D): R^{t}=0.462 min, [M+H]⁺=832.4.

### Step 2: Synthesis of I-152-4

To a solution of I-152-2 (120 mg, 144.23 μmol, 1 eq), I-152-3 (86.13 mg, 144.23 μmol, 1 eq) in DMF (1 mL) was added NMM (72.94 mg, 721.16 μmol, 79.29 μL, 5 eq), EDCI (138.25 mg, 721.16 μmol, 5 eq), HOAt (39.26 mg, 288.46 μmol, 40.35 μL, 2 eq) were added at 25° C. The mixture was stirred at 25° C. for 0.5 hr. The reaction mixture was washed with water (1 mL) and extracted with EA (1 mL*3), the combined organic phase was dried by Na₂SO₄, concentrated under reduced pressure to give a residue. The residue was purified by flash silica gel chromatography (ISCO®; 4 g SepaFlash® Silica Flash Column, Eluent of 0˜50% Ethyl acetate/Petroleum ether @35 mL/min) and the eluent was concentrated to give I-152-4 (110 mg, 77.95 μmol, 54.05% yield, 100% purity) as a white solid. LCMS (Method D): R^{t}=0.457 min, [M+H]⁺=1411.3.

### Step 3: Synthesis of I-152

To a solution of I-152-4 (100 mg, 70.87 μmol, 1 eq) HCl (0.1 M, 1 mL). The mixture was stirred at 100° C. for 1 hr. The reaction mixture was filtered to give the filtrate. The residue was purified by reverse-phase (0.1% of FA) and the eluent was lyophilized to give I-152 (30 mg, 23.63 μmol, 33.35% yield, 95.398% purity) as a white solid. LCMS (Method D): R^{t}=0.357 min, [M+H]⁺=1210.6. ¹H NMR (400 MHz, METHANOL-d4) δ=8.43 (s, 2H), 8.14 (s, 1H), 7.81 (d, J=1.2 Hz, 1H), 7.77-7.69 (m, 2H), 7.51-7.40 (m, 2H), 7.35-7.24 (m, 5H), 7.14 (d, J=3.6 Hz, 1H), 6.65-6.59 (m, 2H), 6.58-6.52 (m, 1H), 4.97 (t, J=6.8 Hz, 1H), 4.60-4.52 (m, 4H), 4.32-4.27 (m, 1H), 4.24-4.17 (m, 2H), 4.16-4.09 (m, 2H), 4.08-4.03 (m, 1H), 4.01-3.92 (m, 1H), 3.83-3.77 (m, 3H), 3.73-3.66 (m, 1H), 3.62 (t, J=12.8 Hz, 2H), 3.57-3.47 (m, 4H), 3.43 (s, 2H), 3.26-3.12 (m, 1H), 2.98-2.86 (m, 4H), 2.86-2.66 (m, 2H), 2.43-2.30 (m, 6H), 2.29-2.17 (m, 2H), 2.07-1.99 (m, 2H), 1.99-1.92 (m, 1H), 1.90-1.79 (m, 4H), 1.79-1.68 (m, 5H), 1.68-1.52 (m, 3H), 1.50-1.39 (m, 3H), 1.32-1.16 (m, 3H), 1.12-1.00 (m, 2H).

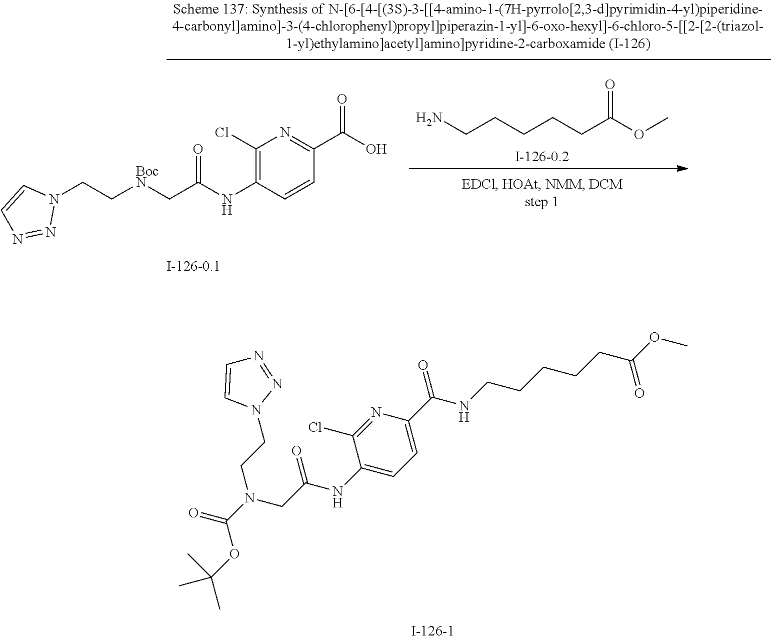

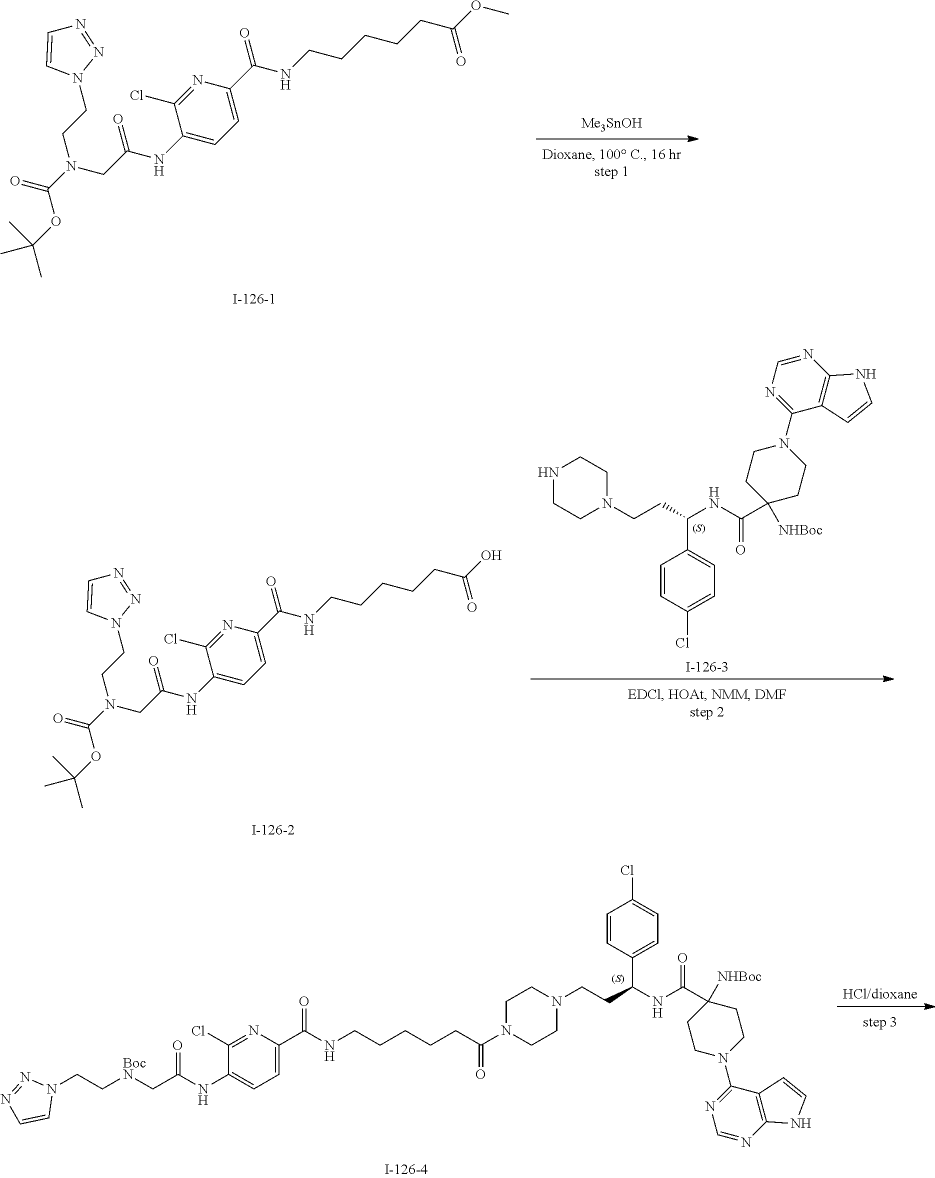

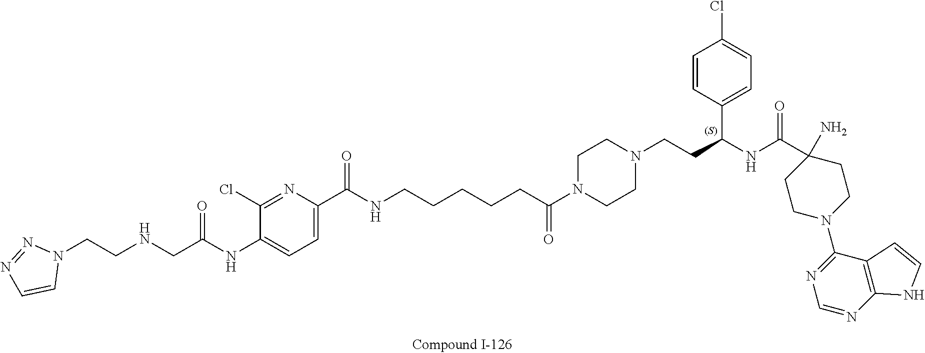

### Step 1: Synthesis of I-126-1

To a solution of I-126-0.1 (100 mg, 235.38 μmol, 1 eq), EDCI (90.25 mg, 470.77 μmol, 2 eq), HOAt (16.02 mg, 117.69 μmol, 16.46 μL, 0.5 eq), NMM (119.04 mg, 1.18 mmol, 129.39 μL, 5 eq) in DCM (1 mL) was added I-126-0.2 (51.31 mg, 282.46 μmol, 1.2 eq, HCl). Then the mixture was stirred at 25° C. for 1 hr. The reaction mixture was poured into water (5 mL) and extracted with DCM (5 mL*2), the organic layer was washed with brine (5 mL) and dried over Na₂SO₄. Concentrated to get the crude product. The crude product was purified by prep-HPLC (column: C18 150×30 mm; mobile phase: [water (FA)-ACN]; gradient: 40%-70% B over 7 min) to get I-126-1 (50 mg, 83.61 μmol, 35.52% yield, 100% purity, FA) as colorless oil. LCMS (Method E): R^{t}=0.503 min, [M+H]⁺=552.3. ¹H NMR (400 MHZ, CHLOROFORM-d) δ=9.13-8.68 (m, 2H), 8.14 (d, J=8.4 Hz, 1H), 7.76-7.50 (m, 3H), 4.62 (br s, 2H), 4.09-3.80 (m, 4H), 3.67 (s, 3H), 3.47-3.45 (m, 2H), 2.35-2.33 (m, 2H), 1.74-1.60 (m, 4H), 1.54-1.30 (m, 11H).

### Step 1: Synthesis of I-126-2

To a solution of I-126-1 (100 mg, 181.15 μmol, 1 eq) in dioxane (2 mL) was added hydroxy (trimethyl) stannane (163.78 mg, 905.76 μmol, 5 eq). Then the mixture was stirred at 100° C. for 16 hrs. The reaction mixture was filtered and the filtrate was concentrated to get I-126-2 (100 mg, 157.44 μmol, 86.91% yield, 84.7% purity) as colorless oil. LCMS (Method E): R^{t}=0.454 min, [M+H]⁺=538.3.

### Step 2: Synthesis of I-126-4

To a solution of I-126-2 (90 mg, 167.29 μmol, 1 eq), EDCI (64.14 mg, 334.58 μmol, 2 eq), HOAt (11.38 mg, 83.64 μmol, 11.70 μL, 0.5 eq), NMM (84.60 mg, 836.44 μmol, 91.96 μL, 5 eq) in DMF (1 mL) was added I-126-3 (119.87 mg, 200.75 μmol, 1.2 eq). Then the mixture was stirred at 25° C. for 1 hr. The reaction mixture was poured into water (5 mL) and extracted with DCM (5 mL*2), the organic layer was washed with brine (5 mL) and dried over Na₂SO₄. Concentrated to get the crude product. The crude product was purified by flash silica gel column chromatography (SiO₂, PE:EA=10:1 to 3:1) to get I-126-4 (90 mg, 65.50 μmol, 39.15% yield, 81.3% purity) as colorless oil. LCMS (Method G): R^{t}=0.658 min, [M+H]⁺=1116.6.

### Step 3: Synthesis of I-126

A solution of I-126-4 (80 mg, 71.61 μmol, 1 eq) in HCl/dioxane (1 mL) was stirred at 25° C. for 1 hr. The reaction mixture was concentrated under vacuum to get the crude product. The crude product was purified by prep-HPLC (column: Phenomenex luna C18 150*25 mm*10 um; mobile phase: [water (HCl)—ACN]; gradient: 1%-25% B over 10 min) to get I-126 (25.26 mg, 26.50 μmol, 37.00% yield, 100% purity, HCl) as white solid. LCMS (Method E): R^{t}=0.371 min, [M+H]⁺=916.5. SFC: R^{t}=1.483 min. 1H NMR (400 MHZ, METHANOL-d4) δ=8.68-8.52 (m, 2H), 8.40 (s, 1H), 8.30 (s, 1H), 8.05 (d, J=8.4 Hz, 1H), 7.47 (d, J=8.4 Hz, 2H), 7.43 (d, J=3.6 Hz, 1H), 7.34 (d, J=8.4 Hz, 2H), 7.02 (d, J=3.6 Hz, 1H), 5.13-5.11 (m, 3H), 4.71-4.52 (m, 3H), 4.35 (s, 2H), 4.21 (br d, J=14.4 Hz, 1H), 4.14-3.98 (m, 2H), 3.91-3.89 (m, 2H), 3.73-3.54 (m, 3H), 3.46-3.36 (m, 3H), 3.32-3.03 (m, 4H), 2.94-2.73 (m, 2H), 2.61-2.27 (m, 5H), 2.14 (br d, J=14.4 Hz, 1H), 1.67-1.65 (m, 4H), 1.53-1.36 (m, 2H).

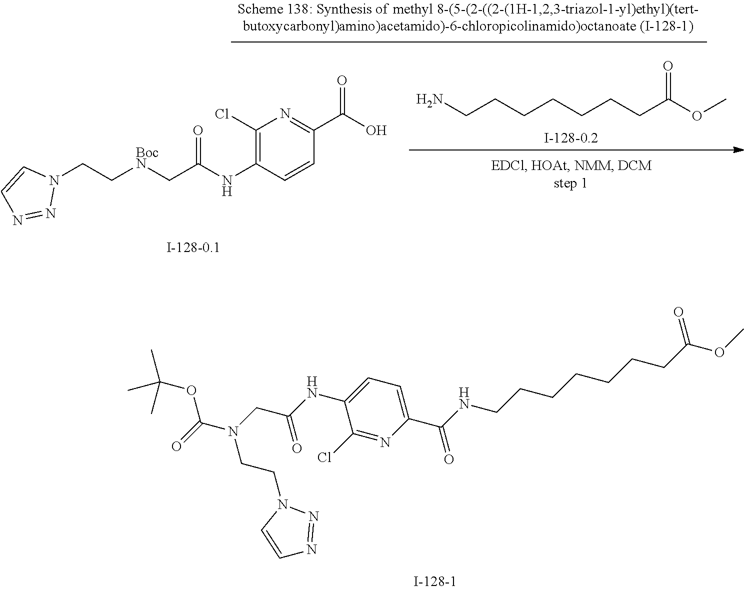

### Step 1: Synthesis of I-128-1

To a solution of I-128-0.1 (1.2 g, 2.82 mmol, 1 eq), EDCI (1.08 g, 5.65 mmol, 2 eq), HOAt (192.23 mg, 1.41 mmol, 197.56 μL, 0.5 eq), NMM (1.43 g, 14.12 mmol, 1.55 mL, 5 eq) in DCM (15 mL) was added I-128.0.2 (710.83 mg, 3.39 mmol, 1.2 eq, HCl). Then the mixture was stirred at 25° C. for 1 hr. The reaction mixture was poured into water (30 mL) and extracted with DCM (30 mL*2), the organic layer was washed with brine (30 mL) and dried over Na₂SO₄. Concentrated to get the crude product. The crude product was purified by flash silica gel column chromatography (SiO₂, PE:EA=10:1 to 1:1) to give I-128-1 (1.1 g, 1.83 mmol, 64.82% yield, 96.55% purity) as a white solid. LCMS (Method E): R^{t}=0.542 min, [M+H]⁺=580.3. ¹H NMR (400 MHZ, CHLOROFORM-d) δ=9.10-8.72 (m, 2H), 8.17 (d, J=8.4 Hz, 1H), 7.77-7.49 (m, 3H), 4.63 (br d, J=2.0 Hz, 2H), 4.10-3.82 (m, 4H), 3.69 (s, 3H), 3.47-3.45 (m, 2H), 2.34-2.32 (m, 2H), 1.66-1.64 (m, 4H), 1.44 (s, 9H), 1.42-1.32 (m, 6H).

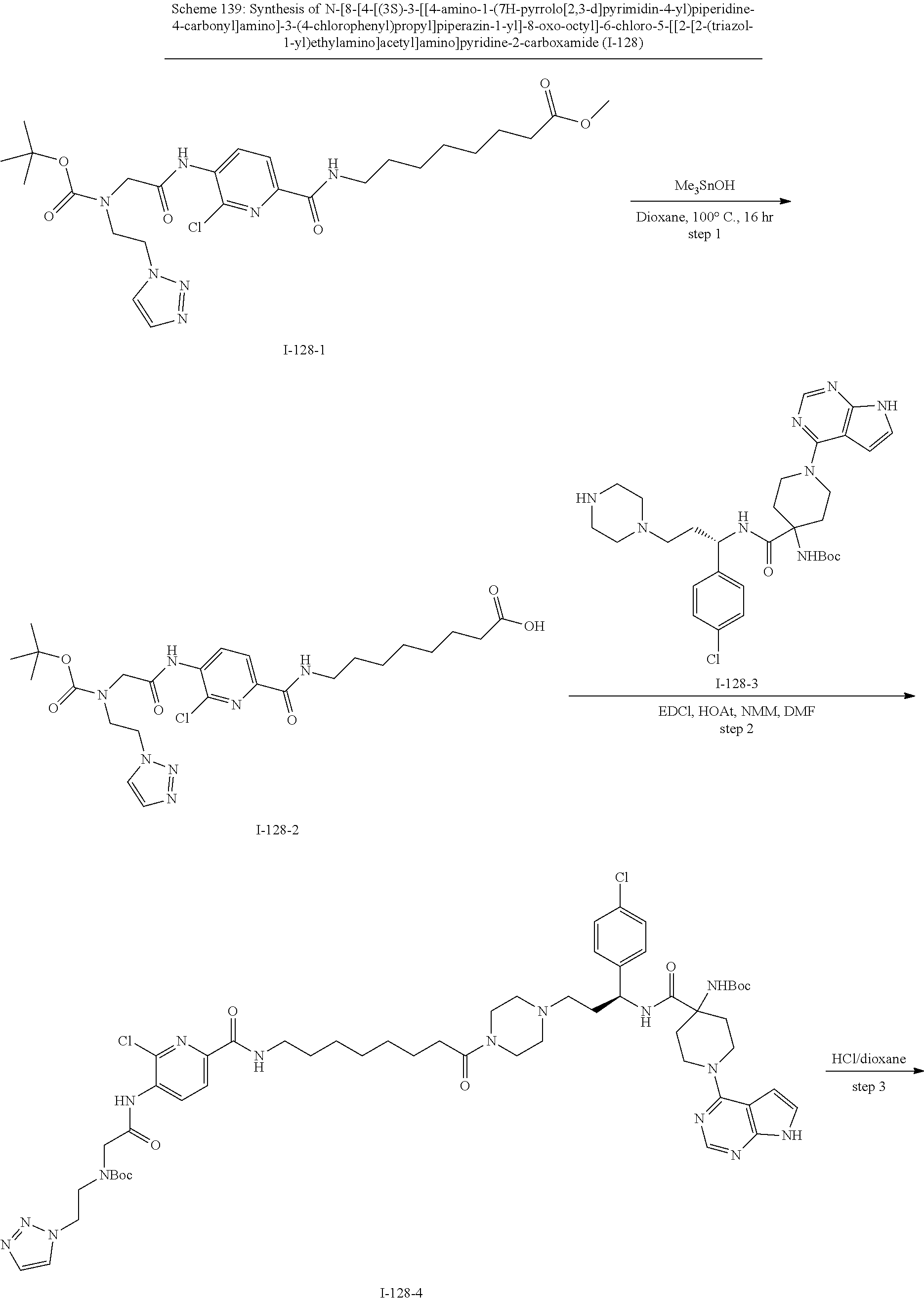

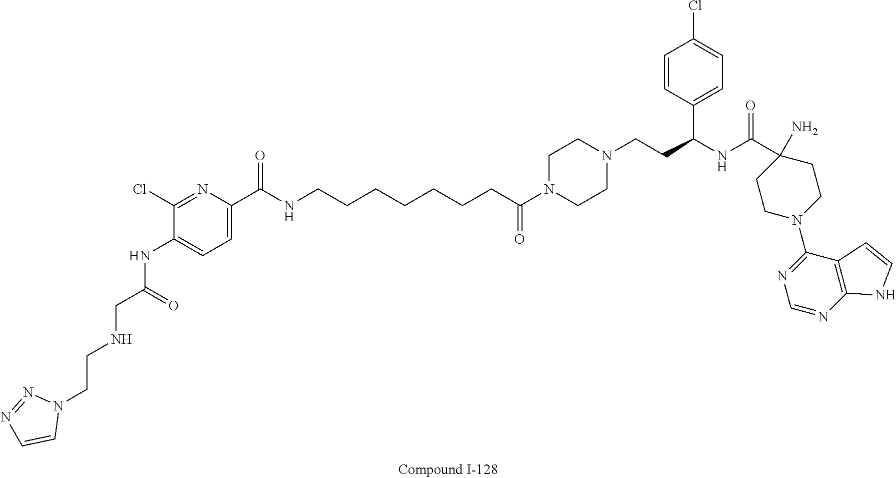 [SMILES: NC1(C(=O)N[C@@H](CCN2CCN(C(=O)CCCCCCCNC(=O)c3ccc(NC(=O)CNCCn4ccnn4)c(Cl)n3)CC2)c2ccc(Cl)cc2)CCN(c2ncnc3[nH]ccc23)CC1]

### Step 1: Synthesis of I-128-2

To a solution of I-128-1 (100 mg, 172.39 μmol, 1 eq) in dioxane (2 mL) was added hydroxy (trimethyl) stannane (155.86 mg, 861.96 μmol, 5 eq). Then the mixture was stirred at 100° C. for 16 hrs. The reaction mixture was filtered and the filtrate was concentrated to get I-128-2 (100 mg, 159.00 μmol, 92.23% yield, 90.0% purity) as colorless oil. LCMS (Method E): R^{t}=0.481 min, [M+H]⁺=566.3.

### Step 2: Synthesis of I-128-4

To a solution of I-128-2 (90 mg, 159.00 μmol, 1 eq), EDCI (60.96 mg, 317.99 μmol, 2 eq), HOAt (10.82 mg, 79.50 μmol, 11.12 μL, 0.5 eq), NMM (80.41 mg, 794.99 μmol, 87.40 μL, 5 eq) in DMF (1 mL) was added I-128-3 (113.93 mg, 190.80 μmol, 1.2 eq). Then the mixture was stirred at 25° C. for 1 hr. The reaction mixture was poured into water (5 mL) and extracted with DCM (5 mL*2), the organic layer was washed with brine (5 mL) and dried over Na₂SO₄. Concentrated to get the crude product. The crude product was purified by flash silica gel column chromatography (SiO₂, PE:EA=10:1 to 3:1) to get I-128-4 (90 mg, 71.36 μmol, 44.88% yield, 90.8% purity) as colorless oil. LCMS (Method G): R^{t}=0.693 min, [M+H]⁺=1146.7.

### Step 3: Synthesis of I-128

A solution of I-128-4 (80 mg, 69.86 μmol, 1 eq) in HCl/dioxane (1 mL) was stirred at 25° C. for 1 hr. The reaction mixture was concentrated under vacuum to get the crude product. The crude product was purified by prep-HPLC (column: Phenomenex luna C18 150*25 mm*10 um; mobile phase: [water (HCl)—ACN]; gradient: 1%-30% B over 10 min) to get I-128 (22.73 mg, 23.16 μmol, 33.15% yield, 100% purity, HCl) as a white solid. LCMS (Method E): R^{t}=0.389 min, [M+H]⁺=944.6. SFC: R^{t}=1.580 min. 1H NMR (400 MHZ, METHANOL-d4) δ=8.62 (d, J=8.4 Hz, 1H), 8.50 (s, 1H), 8.40 (s, 1H), 8.22 (s, 1H), 8.06 (d, J=8.4 Hz, 1H), 7.54-7.40 (m, 3H), 7.35 (d, J=8.4 Hz, 2H), 7.03 (d, J=3.6 Hz, 1H), 5.09-5.07 (m, 3H), 4.72-4.55 (m, 3H), 4.34 (s, 2H), 4.20 (br d, J=14.6 Hz, 1H), 4.05-4.03 (m, 2H), 3.89-3.87 (m, 2H), 3.76-3.53 (m, 3H), 3.43-3.41 (m, 3H), 3.32-3.03 (m, 4H), 2.94-2.69 (m, 2H), 2.56 (br d, J=10.4 Hz, 1H), 2.45-2.43 (m, 2H), 2.40-2.25 (m, 2H), 2.12 (br d, J=14.8 Hz, 1H), 1.73-1.51 (m, 4H), 1.39 (br s, 6H).

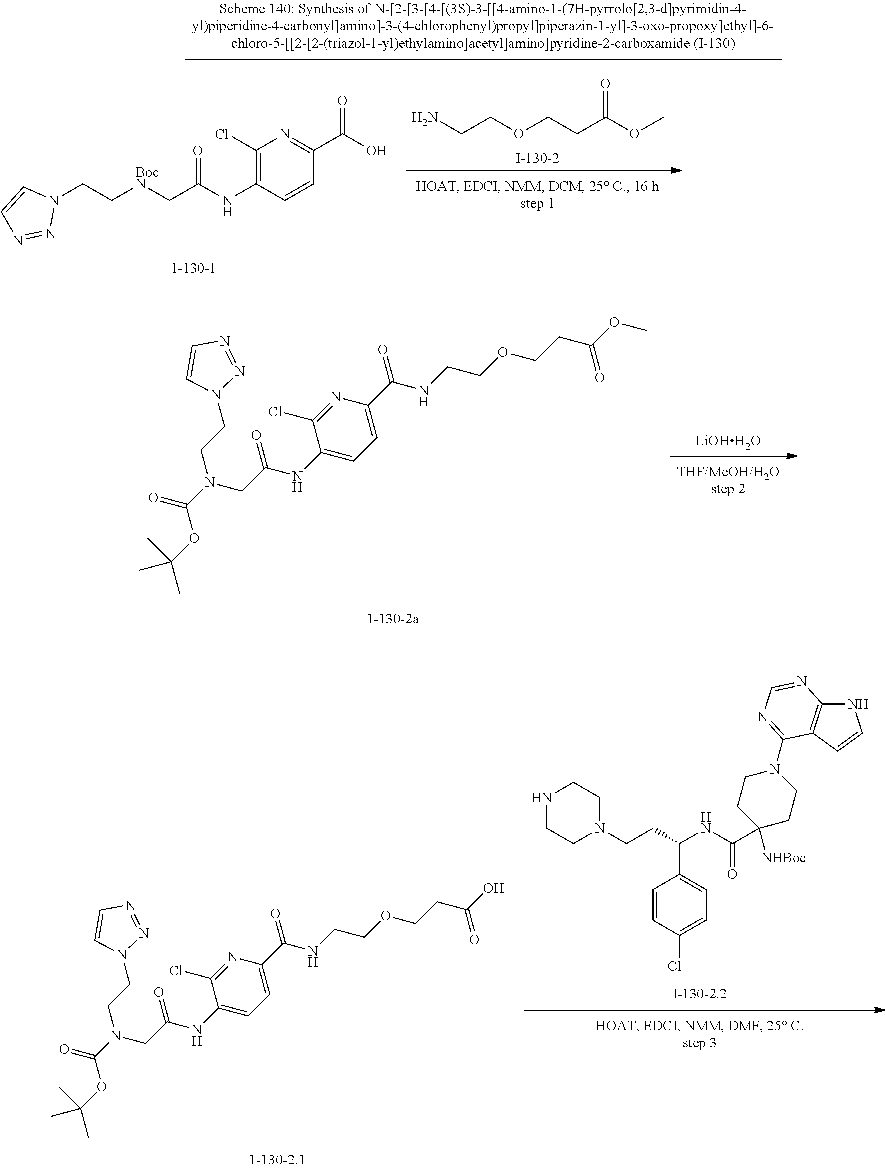

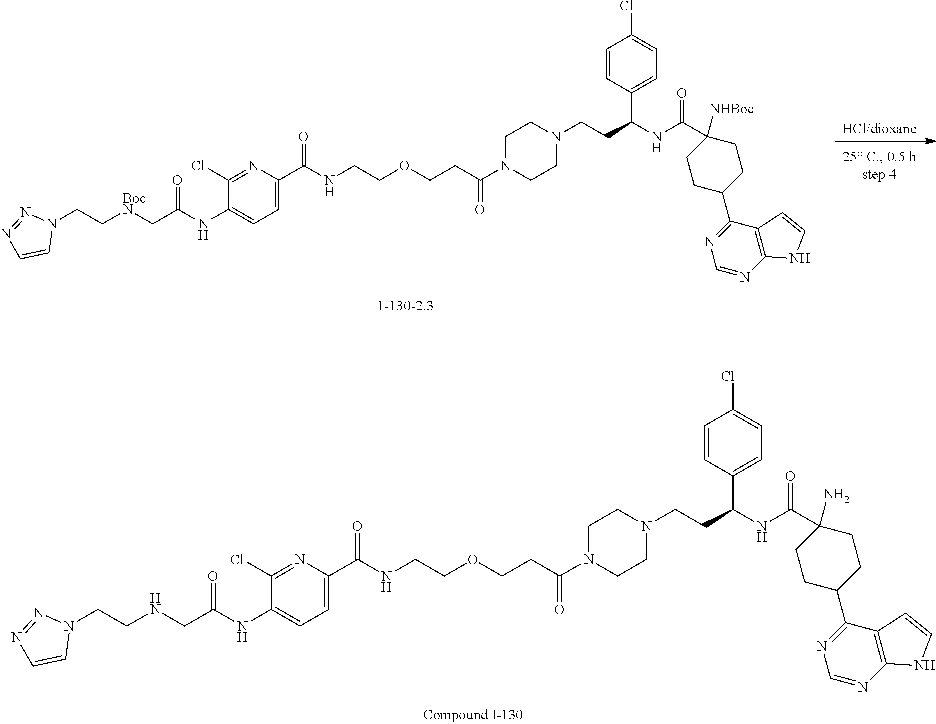 [SMILES: CC(C)(C)OC(=O)NC1(C(=O)N[C@@H](CCN2CCN(C(=O)CCOCCNC(=O)c3ccc(NC(=O)CN(CCn4ccnn4)C(=O)OC(C)(C)C)c(Cl)n3)CC2)c2ccc(Cl)cc2)CCC(c2ncnc3[nH]ccc23)CC1 ; "HCl/dioxane" ; NC1(C(=O)N[C@@H](CCN2CCN(C(=O)CCOCCNC(=O)c3ccc(NC(=O)CNCCn4ccnn4)c(Cl)n3)CC2)c2ccc(Cl)cc2)CCC(c2ncnc3[nH]ccc23)CC1]

### Step 1: Synthesis of I-130-2a

To a solution of I-130-1 (1.2 g, 2.82 mmol, 1 eq) and I-130-2 (623.55 mg, 3.40 mmol, 1.20 eq, HCl) in DCM (10 mL) was added EDCI (1.08 g, 5.65 mmol, 2 eq), NMM (1.43 g, 14.12 mmol, 1.55 mL, 5 eq) and HOAt (192.23 mg, 1.41 mmol, 197.56 μL, 0.5 eq). The mixture was stirred at 25° C. for 16 hrs. The reaction mixture was quenched with water (10 mL). The mixture was extracted with EA (5 mL*3) and dried over anhydrous Na₂SO₄. The mixture filtered and the filtrate was concentrated under vacuum. The residue was purified by flash silica gel chromatography (ISCO®; 40 g SepaFlash® Silica Flash Column, Eluent of 80˜100% Ethyl acetate/Petroleum ether gradient @ 40 mL/min) to afford I-130-2a (880 mg, 1.59 mmol, 56.24% yield, 100% purity) as a white solid. LCMS (Method E): R^{t}=0.466 min, [M+H]⁺=554.3. ¹H NMR (400 MHZ, CHLOROFORM-d) δ=9.02-8.77 (m, 1H), 8.13 (d, J=8.4 Hz, 1H), 7.93 (s, 1H), 7.73-7.51 (m, 2H), 4.82-4.45 (m, 2H), 4.01-3.83 (m, 4H), 3.79-3.75 (m, 2H), 3.70 (s, 3H), 3.67-3.55 (m, 4H), 2.63-2.59 (m, 2H), 1.41 (s, 9H).

### Step 2: Synthesis of I-130-2.1

To a solution of I-130-2a (100 mg, 180.51 μmol, 1 eq) in THF (1 mL) was added LiOH·H₂O (22.72 mg, 541.52 μmol, 3 eq) in H₂O (1 mL) and MeOH (0.3 mL). The mixture was stirred at 0° C. for 0.5 hr. The reaction mixture was concentrated under reduced pressure to remove solvent. The residue was diluted with

HCl (0.1 mL, 1M) and extracted with EA 3 mL (1 mL*3). The combined organic layers were washed with NaCl 6 mL (3 mL*2), and dried over [Na₂SO₄], filtered and concentrated under reduced pressure to give a residue. The residue was purified by prep-HPLC (FA condition) to afford I-130-2.1 (100 mg, crude) as a white solid. LCMS (Method E): R^{t}=0.428 min, [M+H]⁺=540.3. ¹H NMR (400 MHZ, CHLOROFORM-d) δ=9.04-8.93 (m, 1H), 8.86-8.74 (m, 1H), 8.13 (d, J=8.4 Hz, 1H), 8.07-7.98 (m, 1H), 7.74-7.52 (m, 2H), 4.69-4.54 (m, 2H), 4.06-3.87 (m, 4H), 3.80-3.76 (m, 2H), 3.69-3.63 (m, 4H), 2.65-2.61 (m, 2H), 1.43 (s, 9H).

### Step 3: Synthesis of I-130-2.3

To a solution of I-130-2.1 (90 mg, 155.01 μmol, 1 eq) and I-130-2.2 (92.56 mg, 155.01 μmol, 1 eq) in DMF (2 mL) was added EDCI (59.43 mg, 310.02 μmol, 2 eq), NMM (78.39 mg, 775.05 μmol, 85.21 μL, 5 eq) and HOAt (10.55 mg, 77.50 μmol, 10.84 μL, 0.5 eq). The mixture was stirred at 25° C. for 1 hr. The reaction mixture was quenched with water (5 mL). The mixture was extracted with EA (5 mL*3) and dried over anhydrous Na₂SO₄. The mixture filtered and the filtrate was concentrated under vacuum. The residue was purified by prep-HPLC (FA condition) to afford I-130-2.3 (80 mg, 70.77 μmol, 45.66% yield, 99% purity) as a white solid. LCMS (Method G): R^{t}=0.601 min, [M+H]⁺=1118.5.

### Step 4: Synthesis of I-130

To a solution of I-130-2.3 (80 mg, 71.49 μmol, 1 eq) was added HCl/dioxane (1 mL). The mixture was stirred at 25° C. for 0.5 hr. The reaction mixture was concentrated under the pressure to give the product. The product was diluted with H₂O (5 mL), and lyophilized to give I-130 (64 mg, 66.99 μmol, 93.71% yield, 100% purity, HCl) as a white solid. LCMS (Method E): R^{t}=0.368 min, [M+H]⁺=918.6. SFC: R^{t}=1.197 min. ¹H NMR (400 MHZ, METHANOL-d4) δ=8.60 (d, J=8.4 Hz, 1H), 8.39 (s, 1H), 8.28-8.16 (m, 1H), 8.05 (d, J=8.4 Hz, 1H), 7.98-7.87 (m, 1H), 7.46-7.33 (m, 5H), 6.99 (d, J=3.6 Hz, 1H), 5.07-5.03 (m, 1H), 4.96 (s, 2H), 4.66-4.58 (m, 2H), 4.29 (s, 2H), 4.01-3.91 (m, 2H), 3.84-3.74 (m, 4H), 3.67-3.51 (m, 10H), 3.28-2.71 (m, 8H), 2.64-2.47 (m, 2H), 2.36-2.22 (m, 2H), 2.11-2.00 (m, 2H).

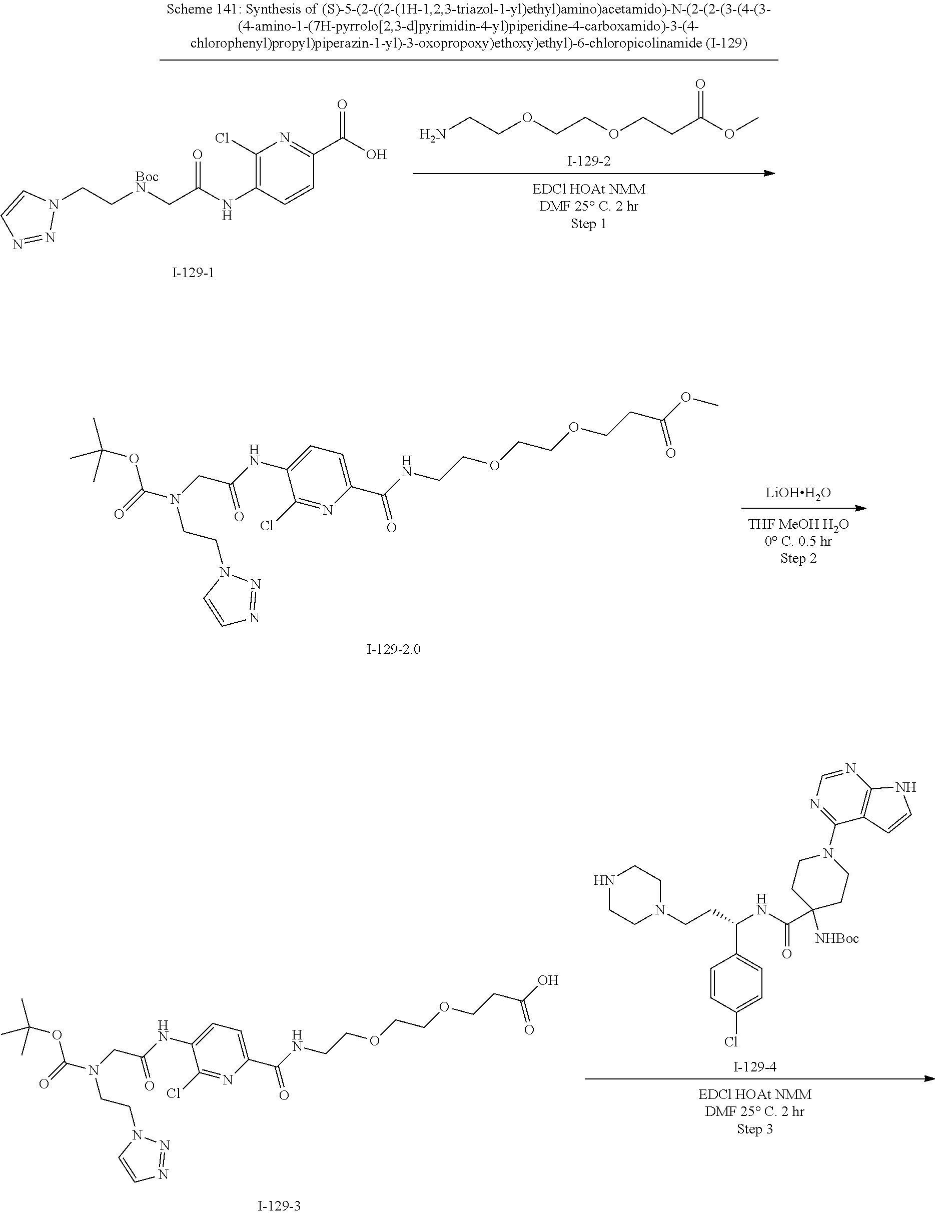

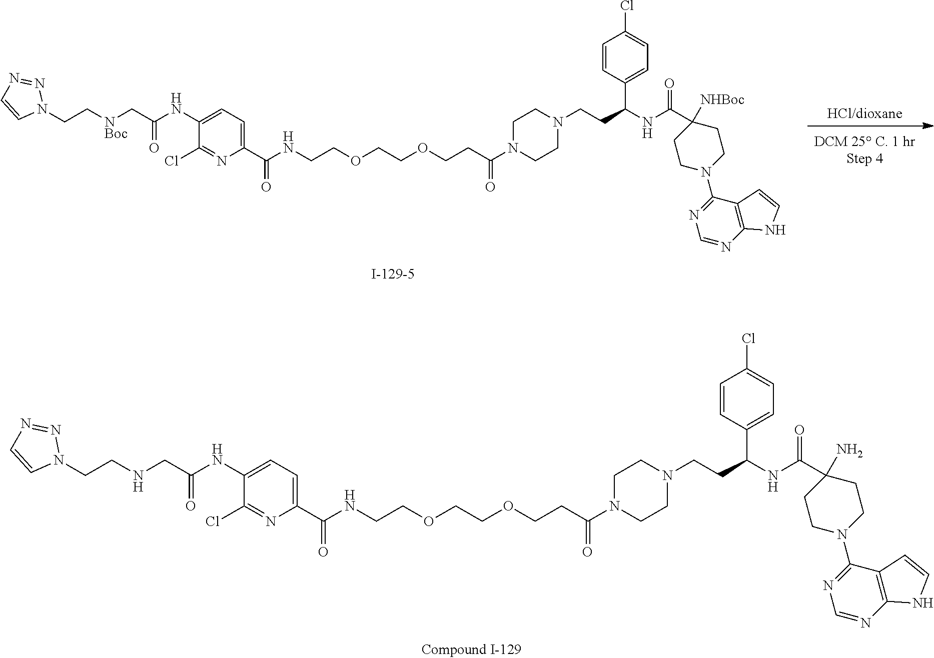 [SMILES: CC(C)(C)OC(=O)NC1(C(=O)N[C@@H](CCN2CCN(C(=O)CCOCCOCCNC(=O)c3ccc(NC(=O)CN(CCn4ccnn4)C(=O)OC(C)(C)C)c(Cl)n3)CC2)c2ccc(Cl)cc2)CCN(c2ncnc3[nH]ccc23)CC1 ; "HCl/dioxane" ; NC1(C(=O)N[C@@H](CCN2CCN(C(=O)CCOCCOCCNC(=O)c3ccc(NC(=O)CNCCn4ccnn4)c(Cl)n3)CC2)c2ccc(Cl)cc2)CCN(c2ncnc3[nH]ccc23)CC1]

### Step 1: Synthesis of I-129-2.0

To a solution of I-129-1 (150 mg, 353.08 μmol, 1 eq) in DMF (2 mL) was added HOAt (48.06 mg, 353.08 μmol, 49.39 μL, 1 eq), EDCI (135.37 mg, 706.15 μmol, 2 eq), NMM (178.56 mg, 1.77 mmol, 194.09 μL, 5 eq) and I-129-2 (87.77 mg, 459.00 μmol, 1.3 eq). The mixture was stirred at 25° C. for 1 hr. The reaction mixture was quenched by addition water 20 mL at 25° C., and then extracted with EA 15 mL (5 mL*3). Then dried over Na₂SO₄, filtered and concentrated under reduced pressure to give a residue. The residue was purified by prep-HPLC (column: Phenomenex Luna C18 150*25 mm*10 um; mobile phase: [water (FA)-ACN]; gradient: 50%-80% B over 10 min), the target peak was concentrated and lyophilized to afford I-129-2.0 (120 mg, 200.65 μmol, 56.83% yield, 100% purity) as yellow oil. LCMS (Method E): R^{t}=0.468 min, [M+H]⁺=598.4. ¹H NMR (400 MHZ, CHLOROFORM-d) δ=9.10-8.70 (m, 2H), 8.15-8.12 (d, J=8.4 Hz, 1H), 8.01 (s, 1H), 7.70-7.53 (m, 2H), 4.60 (s, 2H), 4.02-3.86 (m, 4H), 3.79 (m, 2H), 3.68 (s, 3H), 3.67-3.62 (m, 8H), 2.62 (m, 2H), 1.41 (s, 9H).

### Step 2: Synthesis of I-129-3

To a solution of I-129-2.0 (100 mg, 167.21 μmol, 1 eq) in THF (1 mL), H₂O (1 mL), and MeOH (0.2 mL) was added LiOH·H₂O (35.08 mg, 836.05 μmol, 5 eq). The mixture was stirred at 0° C. for 0.5 hr. The mixture poured into water 10 mL, then was added in HCl (1 M) to PH=6 and extracted with EA (10 mL*3). Then dried over Na₂SO₄, filtered and concentrated under reduced pressure to give a residue. The residue was purified by reversed phase (NH3-H₂O condition), then concentrated to remove organic solvents and lyophilized to give I-129-3 (95 mg, 162.67 μmol, 97.28% yield, 100% purity) as white solid. LCMS: R^{t}=0.301 min, [M+H]⁺=584.3.

### Step 3: Synthesis of I-129-5

To a solution of I-129-3 (80 mg, 136.98 μmol, 1 eq) in DMF (0.5 mL) was added HOAt (18.64 mg, 136.98 μmol, 19.16 μL, 1 eq), EDCI (52.52 mg, 273.96 μmol, 2 eq), NMM (69.28 mg, 684.91 μmol, 75.30 μL, 5 eq) and I-129-4 (98.16 mg, 164.38 μmol, 1.2 eq). The mixture was stirred at 25° C. for 2 hr. The reaction mixture was quenched by addition water 5 mL at 25° C., and then extracted with EA 15 mL (5 mL*3). Then dried over Na₂SO₄, filtered and concentrated under reduced pressure to give I-129-5 (130 mg, 96.12 μmol, 70.17% yield, 86% purity) as yellow solid. LCMS (Method E): R^{t}=0.451 min, [M+H]⁺=1163.0.

### Step 4: Synthesis of I-129

To a solution of I-129-5 (130 mg, 111.76 μmol, 1 eq) in DCM (2 mL) was added HCl/dioxane (4 M, 2 mL, 71.58 eq). The mixture was stirred at 25° C. for 1 hr. The reaction mixture was concentrated under reduced pressure to give a residue. The residue was purified by prep-HPLC (column: Phenomenex luna C18 150*25 mm*10 um; mobile phase: [water (HCl)-ACN]; gradient: 1%-25% B over 10 min), the target peak was concentrated and lyophilized to afford I-129 (70.76 mg, 73.07 μmol, 65.37% yield, 99.43% purity) as off-white solid. LCMS (Method E): R^{t}=0.372 min, [M+H]⁺=962.4. SFC: R^{t}=1.263 min, ee %=100%. ¹H NMR (400 MHZ, METHANOL-d4) δ=8.59-8.57 (d, J=8.4 Hz, 1H), 8.50 (d, J=0.8 Hz, 1H), 8.38 (s, 1H), 8.22 (d, J=0.8 Hz, 1H), 8.05-8.02 (d, J=8.4 Hz, 1H), 7.45-7.43 (d, J=8.4 Hz, 2H), 7.40 (d, J=3.6 Hz, 1H), 7.33-7.31 (d, J=8.4 Hz, 2H), 6.99 (d, J=3.6 Hz, 1H), 5.10-5.04 (m, 3H). 4.68-4.53 (m, 3H), 4.36-4.27 (m, 2H), 4.22-4.18 (d, J=14.4 Hz, 1H), 4.08-3.94 (m, 2H), 3.87 (m, 2H), 3.74 (m, 2H), 3.69-3.46 (m, 12H), 3.25 (m, 1H), 3.17-2.98 (m, 3H), 2.91-2.68 (m, 3H), 2.64-2.46 (m, 2H), 2.39-2.20 (m, 2H), 2.12-2.09 (d, J=14.4 Hz, 1H).

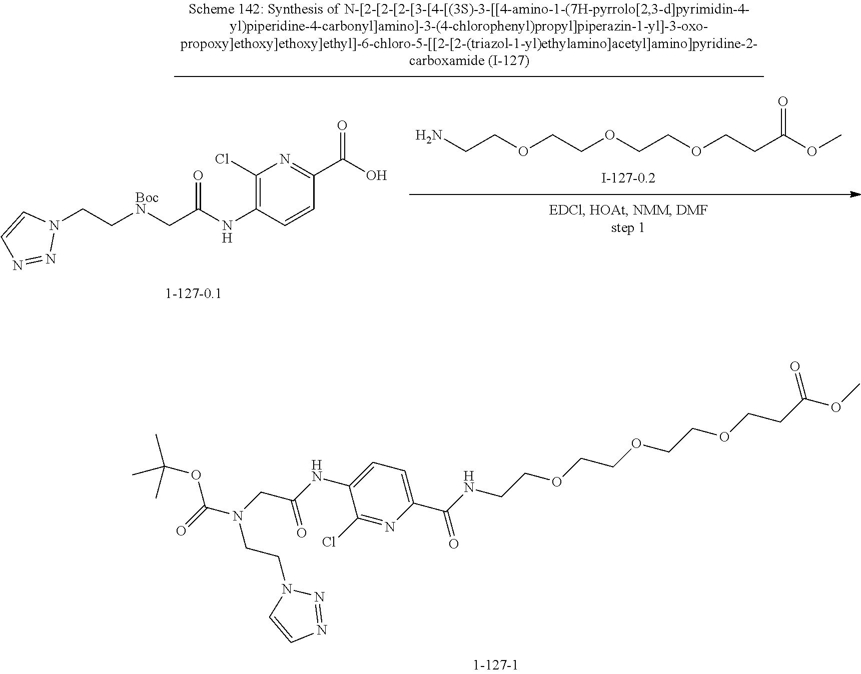 [SMILES: CC(C)(C)OC(=O)N(CCn1ccnn1)CC(=O)Nc1ccc(C(=O)O)nc1Cl ; COC(=O)CCOCCOCCOCCN ; COC(=O)CCOCCOCCOCCNC(=O)c1ccc(NC(=O)CN(CCn2ccnn2)C(=O)OC(C)(C)C)c(Cl)n1]

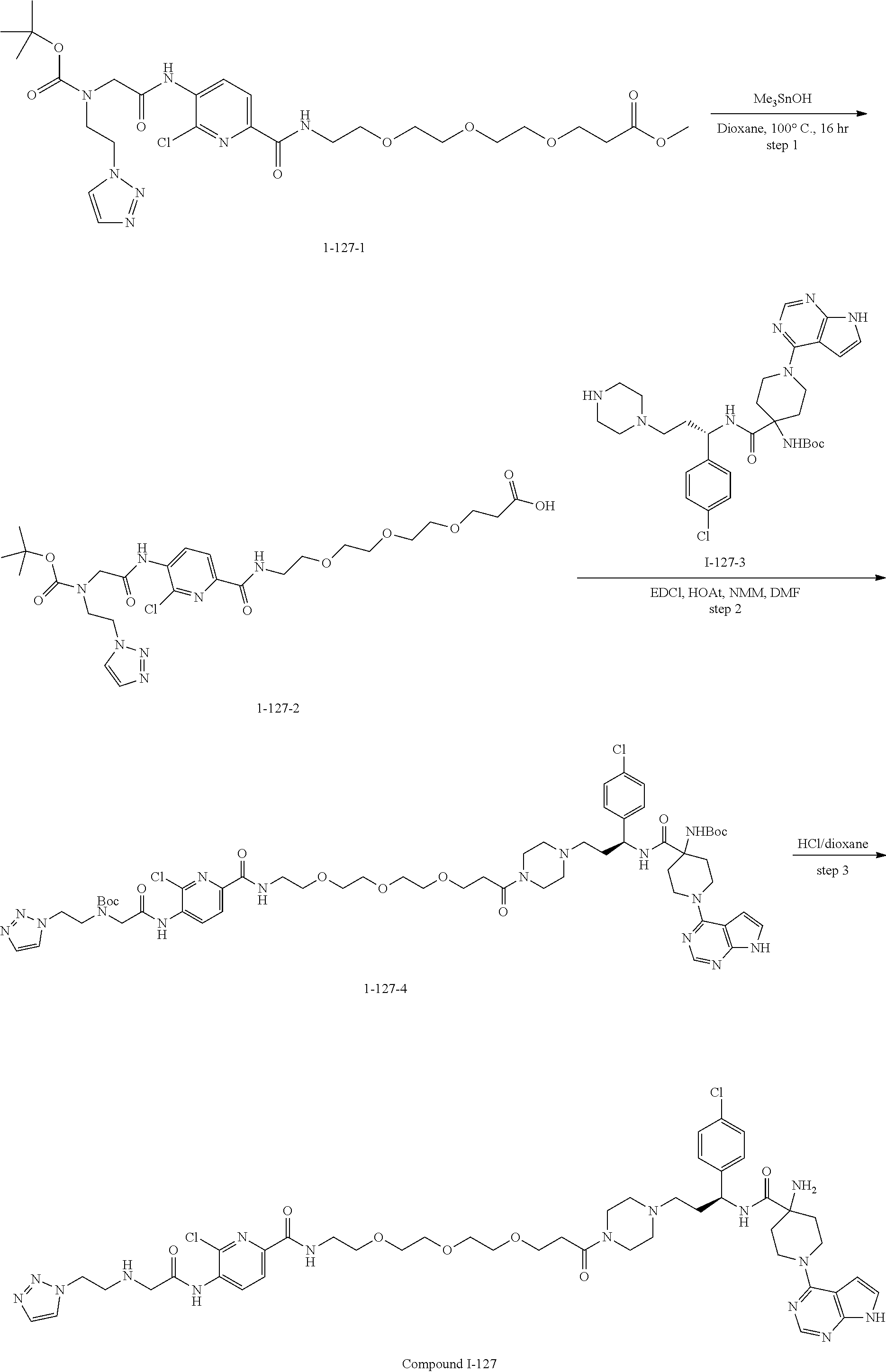

### Step 1: Synthesis of I-127-1

To a solution of I-127-0.1 (1.2 g, 2.82 mmol, 1 eq), EDCI (1.08 g, 5.65 mmol, 2 eq), HOAt (192.23 mg, 1.41 mmol, 197.56μ, 0.5 eq), NMM (1.43 g, 14.12 mmol, 1.55 mL, 5 eq) in DCM (15 mL) was added I-127-0.2 (921.06 mg, 3.39 mmol, 1.2 eq, HCl). Then the mixture was stirred at 25° C. for 1 hr. The reaction mixture was poured into water (30 mL) and extracted with DCM (30 mL*2), the organic layer was washed with brine (30 mL) and dried over Na₂SO₄. Concentrated to get the crude product. The crude product was purified by prep-HPLC (column: YMC-Actus Triart C18 150*30 mm*7 um; mobile phase: [water (FA)-ACN]; gradient: 33%-63% B over 10 min) and then lyophilized to afford I-127-1 (1.1 g, 1.71 mmol, 60.65% yield, 100% purity) as colorless oil. LCMS (Method E): R^{t}=0.467 min, [M+H]⁺=642.3. ¹H NMR (400 MHz, METHANOL-d4) δ=8.21-7.98 (m, 2H), 7.76 (br d, J=9.6 Hz, 1H), 4.68 (br d, J=4.8 Hz, 2H), 4.24-4.05 (m, 2H), 3.88 (br s, 2H), 3.81-3.52 (m, 17H), 2.57-2.55 (m, 2H), 1.37 (br d, J=12.0 Hz, 9H).

### Step 1: Synthesis of I-127-2

To a solution of I-127-1 (100 mg, 155.74 μmol, 1 eq) in dioxane (2 mL) was added hydroxy (trimethyl) stannane (140.80 mg, 778.70 μmol, 5 eq). Then the mixture was stirred at 100° C. for 16 hrs. The reaction mixture was filtered and the filtrate was concentrated to get I-127-2 (100 mg, 120.69 μmol, 77.49% yield, 75.8% purity) as colorless oil. LCMS (Method E): R^{t}=0.436 min, [M+H]⁺=628.3.

### Step 2: Synthesis of I-127-4

To a solution of I-127-2 (90 mg, 143.30 μmol, 1 eq), EDCI (54.94 mg, 286.59 μmol, 2 eq), HOAt (9.75 mg, 71.65 μmol, 10.02 μL, 0.5 eq), NMM (72.47 mg, 716.48 μmol, 78.77 μL, 5 eq) in DMF (1 mL) was added I-127-3 (102.68 mg, 171.95 μmol, 1.2 eq). Then the mixture was stirred at 25° C. for 1 hr. The reaction mixture was poured into water (5 mL) and extracted with DCM (5 mL*2), the organic layer was washed with brine (5 mL) and dried over Na₂SO₄. Concentrated to get the crude product. The crude product was purified by flash silica gel column chromatography (SiO₂, PE:EA=10:1 to 3:1) to get I-127-4 (90 mg, 73.81 μmol, 51.51% yield, 99.0% purity) as colorless oil. LCMS (Method G): R^{t}=0.636 min, [M+H]⁺=1208.7.

### Step 3: Synthesis of I-127

A solution of I-127-4 (80 mg, 66.27 μmol, 1 eq) in HCl/dioxane (1 mL) was stirred at 25° C. for 1 hr. The reaction mixture was concentrated under vacuum to get the crude product. The crude product was purified by prep-HPLC (column: Phenomenex luna C18 150*25 mm*10 um; mobile phase: [water (HCl)—ACN]; gradient: 1%-25% B over 10 min) to get I-127 (12.32 mg, 11.81 μmol, 17.82% yield, 100% purity, HCl) as a white solid. LCMS (Method E): R^{t}=0.369 min, [M+H]⁺=1006.5. SFC: R^{t}=1.367 min. ¹H NMR (400 MHZ, METHANOL-d4) δ=8.61 (d, J=8.4 Hz, 1H), 8.40 (s, 1H), 8.32 (s, 1H), 8.14-7.96 (m, 2H), 7.53-7.39 (m, 3H), 7.35 (d, J=8.4 Hz, 2H), 7.02 (d, J=4.0 Hz, 1H), 5.11-4.97 (m, 4H), 4.63 (br d, J=14.0 Hz, 3H), 4.32 (s, 2H), 4.26-4.24 (m, 1H), 4.01-3.99 (m, 2H), 3.85-3.83 (m, 2H), 3.77-3.54 (m, 17H), 3.27-3.00 (m, 4H), 2.93-2.69 (m, 3H), 2.65-2.49 (m, 2H), 2.43-2.21 (m, 2H), 2.14-2.06 (m, 1H).

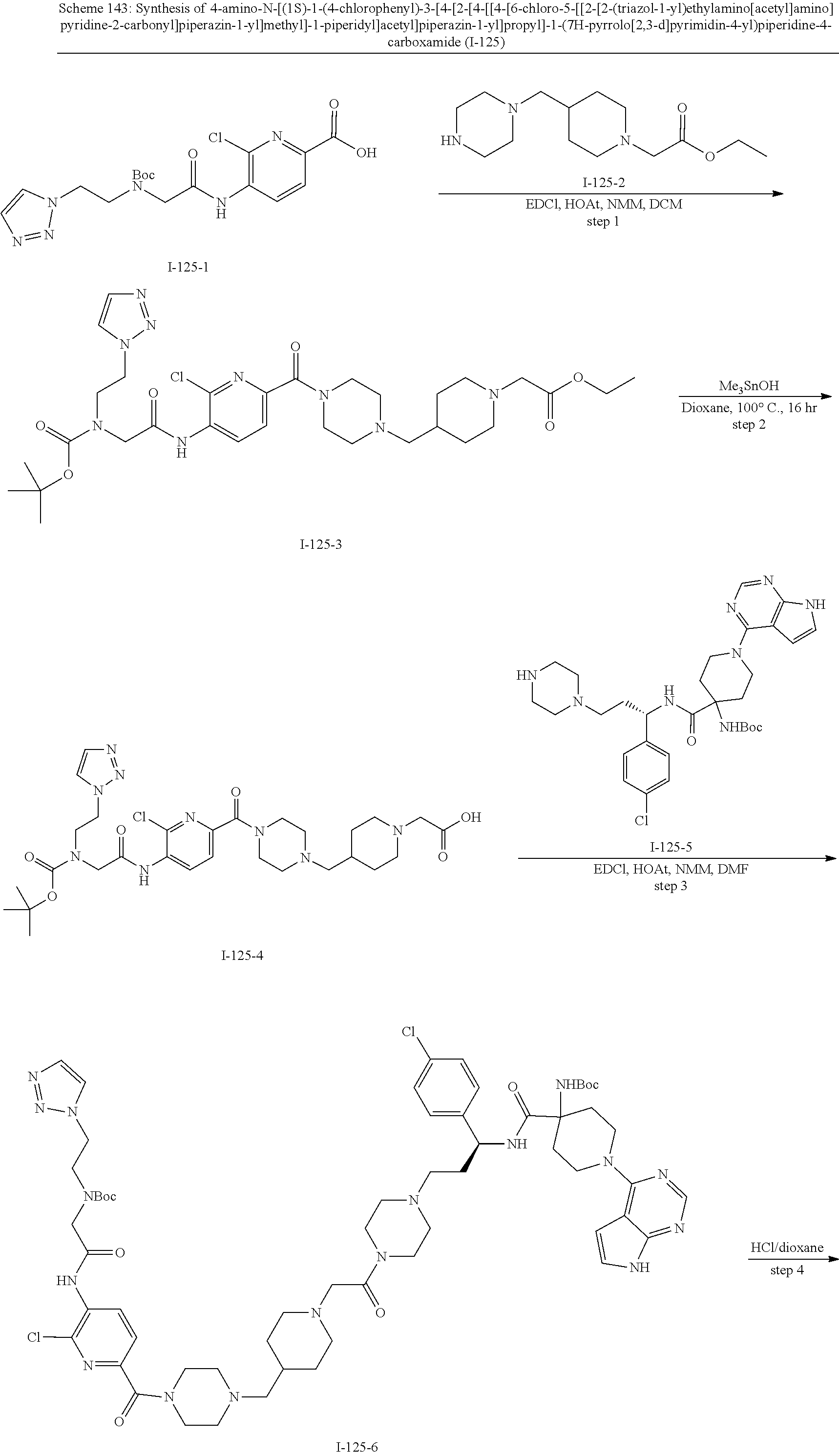

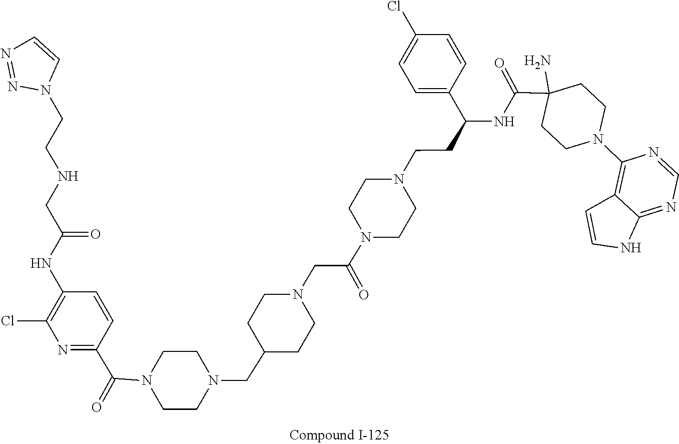 [SMILES: NC1(C(=O)N[C@@H](CCN2CCN(C(=O)CN3CCC(CN4CCN(C(=O)c5ccc(NC(=O)CNCCn6ccnn6)c(Cl)n5)CC4)CC3)CC2)c2ccc(Cl)cc2)CCN(c2ncnc3[nH]ccc23)CC1]

### Step 1: Synthesis of I-125-3

To a solution of I-125-1 (1.2 g, 2.82 mmol, 1 eq), EDCI (1.08 g, 5.65 mmol, 2 eq), HOAt (192.23 mg, 1.41 mmol, 197.56 μL, 0.5 eq), NMM (1.43 g, 14.12 mmol, 1.55 mL, 5 eq) in DCM (15 mL) was added I-125-2 (1.04 g, 3.39 mmol, 1.2 eq, HCl). Then the mixture was stirred at 25° C. for 1 hr. The reaction mixture was poured into water (30 mL) and extracted with DCM (30 mL*2), the organic layer was washed with brine (30 mL) and dried over Na₂SO₄. Concentrated to get the crude product. The crude product was purified by flash silica gel column chromatography (SiO₂, PE:EA=10:1 to 3:1) to get I-125-3 (1.1 g, 1.52 mmol, 53.92% yield, 100% purity, FA) as colorless oil. LCMS (Method E): R^{t}=0.380 min, [M+H]⁺=676.3. ¹H NMR (400 MHZ, METHANOL-d4) δ=8.68-8.44 (m, 1H), 8.33 (s, 1H), 8.13 (br d, J=11.2 Hz, 1H), 7.83-7.59 (m, 2H), 4.68 (br s, 2H), 4.29-4.27 (m, 2H), 4.20-4.05 (m, 2H), 3.97-3.74 (m, 6H), 3.65 (br s, 2H), 3.41 (br d, J=12.0 Hz, 2H), 2.81-2.79 (m, 2H), 2.72-2.51 (m, 4H), 2.40 (br d, J=7.2 Hz, 2H), 1.99 (br d, J=13.2 Hz, 2H), 1.86-1.84 (m, 1H), 1.55-1.28 (m, 14H).

### Step 2: Synthesis of I-125-4

To a solution of I-125-3 (100 mg, 147.88 μmol, 1 eq) in dioxane (2 mL) was added hydroxy (trimethyl) stannane (133.70 mg, 739.42 μmol, 5 eq). Then the mixture was stirred at 100° C. for 16 hrs. The reaction mixture was filtered, and the filtrate was concentrated to get I-125-4 (100 mg, 141.94 μmol, 95.98% yield, 92.0% purity) as colorless oil. LCMS (Method E): R^{t}=0.367 min, [M+H]⁺=648.4.

### Step 3: Synthesis of I-125-6

To a solution of I-125-4 (90 mg, 138.86 μmol, 1 eq), EDCI (53.24 mg, 277.71 μmol, 2 eq), HOAt (9.45 mg, 69.43 μmol, 9.71 μL, 0.5 eq), NMM (70.22 mg, 694.28 μmol, 76.33 μL, 5 eq) in DMF (1 mL) was added I-125-5 (99.50 mg, 166.63 μmol, 1.2 eq). Then the mixture was stirred at 25° C. for 1 hr. The reaction mixture was poured into water (5 mL) and extracted with DCM (5 mL*2), the organic layer was washed with brine (5 mL) and dried over Na₂SO₄. Concentrated to get the crude product. The crude product was purified by flash silica gel column chromatography (SiO₂, PE:EA=10:1 to 3:1) to get I-125-6 (90 mg, 49.87 μmol, 35.91% yield, 68.0% purity) as colorless oil. LCMS (Method G): R^{t}=0.661 min, [M+H]⁺=1228.6.

### Step 4: Synthesis of I-125

A solution of I-125-6 (80 mg, 65.18 μmol, 1 eq) in HCl/dioxane (1 mL) was stirred at 25° C. for 1 hr. The reaction mixture was concentrated to get the crude product. The crude product was purified by prep-HPLC (column: Phenomenex luna C18 150*25 mm*10 um; mobile phase: [water (HCl)-ACN]; gradient: 1%-20% B over 10 min) and SFC separation (column: DAICEL CHIRALCEL OD (250 mm*30 mm, 10 um); mobile phase: [CO₂-EtOH (0.1% NH₃H₂O)]: B %: 75%, isocratic elution mode) to get I-125 (13.40 mg, 12.60 μmol, 19.33% yield, 100% purity, HCl) as a white solid. LCMS (Method E): R^{t}=0.340 min, [M+H]⁺=1026.6. SFC: R^{t}=2.284 min. ¹H NMR (400 MHz, METHANOL-d4) δ=8.62 (d, J=8.4 Hz, 1H), 8.49-8.31 (m, 2H), 8.10 (d, J=1.2 Hz, 1H), 7.82 (d, J=8.4 Hz, 1H), 7.58-7.42 (m, 3H), 7.36 (d, J=8.4 Hz, 2H), 7.05-7.03 (d, J=4.0 Hz, 1H), 5.13-4.99 (m, 4H), 4.87-4.35 (m, 7H), 4.32 (s, 2H), 4.12-3.94 (m, 3H), 3.93-3.56 (m, 10H), 3.54-3.36 (m, 4H), 3.31-3.19 (m, 6H), 2.94 (s, 1H), 2.91-2.70 (m, 2H), 2.56 (br s, 1H), 2.47-2.07 (m, 6H), 1.79-1.77 (m, 1H), 1.34-1.32 (m, 1H).

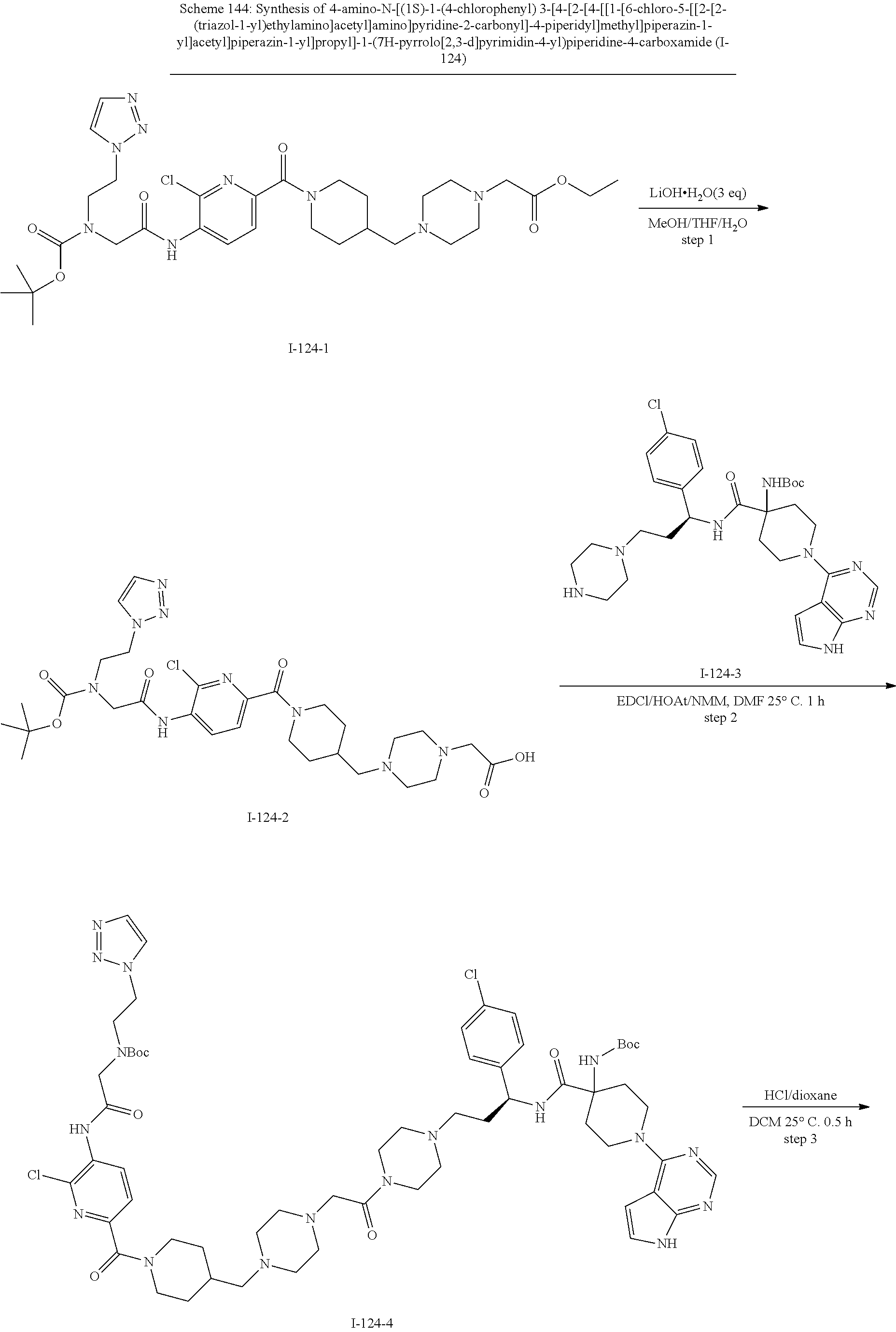

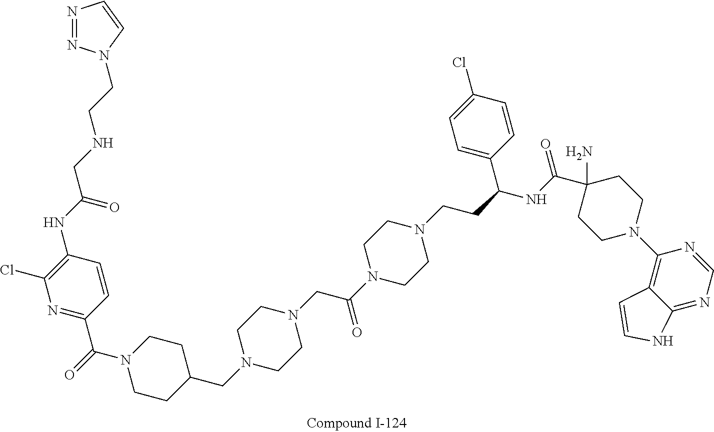 [SMILES: NC1(C(=O)N[C@@H](CCN2CCN(C(=O)CN3CCN(CC4CCN(C(=O)c5ccc(NC(=O)CNCCn6ccnn6)c(Cl)n5)CC4)CC3)CC2)c2ccc(Cl)cc2)CCN(c2ncnc3[nH]ccc23)CC1]

### Step 1: Synthesis of I-124-2

To a solution of I-124-1 (120 mg, 177.46 μmol, 1 eq) in H₂O (1 mL), MeOH (1 mL) and THF (1 mL) was added LiOH·H₂O (22.34 mg, 532.38 μmol, 3 eq). The mixture was stirred at 0° C. for 0.5 hr. The reaction was diluted saturated aqueous citric acid adjust pH=6, filtered under reduced pressure to give a residue. The residue was purified by reverse-phase HPLC (0.1% NH₃·H₂O condition). The eluent was concentrated under reduced pressure to remove ACN, then lyophilized to afford I-124-2 (80 mg, 123.43 μmol, 69.55% yield, 100% purity) as a white solid. LCMS (Method G): R^{t}=0.301 min, [M+H]⁺=648.4.

### Step 2: Synthesis of I-124-4

To a solution of I-124-2 (60 mg, 92.57 μmol, 1 eq) and I-124-3 (55.28 mg, 92.57 μmol, 1 eq) in DMF (0.5 mL) was added EDCI (53.24 mg, 277.71 μmol, 3 eq), NMM (93.64 mg, 925.71 μmol, 101.78 μL, 10 eq) and HOAt (18.90 mg, 138.86 μmol, 19.42 μL, 1.5 eq). The mixture was stirred at 25° C. for 1 hr. The reaction mixture was filtered give a residue. The residue was purified by reverse-phase HPLC (0.1% NH₃·H₂O condition). The eluent was concentrated under reduced pressure to remove ACN, then lyophilized to afford I-124-4 (75 mg, 61.11 μmol, 66.01% yield, N/A purity) as a white solid. LCMS (Method G): R^{t}=0.604 min, [M+H]⁺=1226.7.

### Step 3: Synthesis of I-124

The mixture of I-124-4 (60 mg, 48.89 μmol, 1 eq) in DCM (1 mL) and 4M HCl/dioxane (0.2 mL) was stirred at 25° C. for 0.5 hr. The reaction was concentrated under reduced pressure to give a residue. The residue was purified by prep-HPLC (column: Waters Xbridge 150*25 mm*5 um; mobile phase: [water (NH₄HCO₃)-ACN]; gradient: 20%-50% B over 9 min). The eluent was concentrated under reduced pressure to remove ACN, then lyophilized to afford I-124 (31 mg, 29.87 μmol, 61.11% yield, 98.975% purity) as a white solid. LCMS (Method G): R^{t}=0.523 min, [M+H]⁺=1026.7. SFC: R^{t}=1.384 min. 1H NMR (400 MHZ, METHANOL-d4) δ=8.78 (d, J=8.4 Hz, 1H), 8.14 (s, 1H), 8.07 (d, J=0.8 Hz, 1H), 7.76 (d, J=0.8 Hz, 1H), 7.58 (d, J=8.4 Hz, 1H), 7.34 (s, 4H), 7.13 (d, J=3.6 Hz, 1H), 6.63 (d, J=3.6 Hz, 1H), 5.01-4.98 (m, 1H), 4.93-4.89 (m, 1H), 4.65-4.58 (m, 3H), 4.56-4.46 (m, 2H), 3.86 (d, J=13.2 Hz, 1H), 3.72-3.60 (m, 6H), 3.48 (s, 2H), 3.26-3.23 (m, 2H), 3.21 (s, 2H), 3.16-3.10 (m, 1H), 2.91-2.85 (m, 1H), 2.60-2.43 (m, 10H), 2.42-2.34 (m, 3H), 2.25 (d, J=6.8 Hz, 2H), 2.23-2.12 (m, 2H), 2.11-1.95 (m, 2H), 1.95-1.85 (m, 2H), 1.78 (d, J=13.2 Hz, 1H), 1.68-1.53 (m, 2H), 1.34-1.19 (m, 2H).

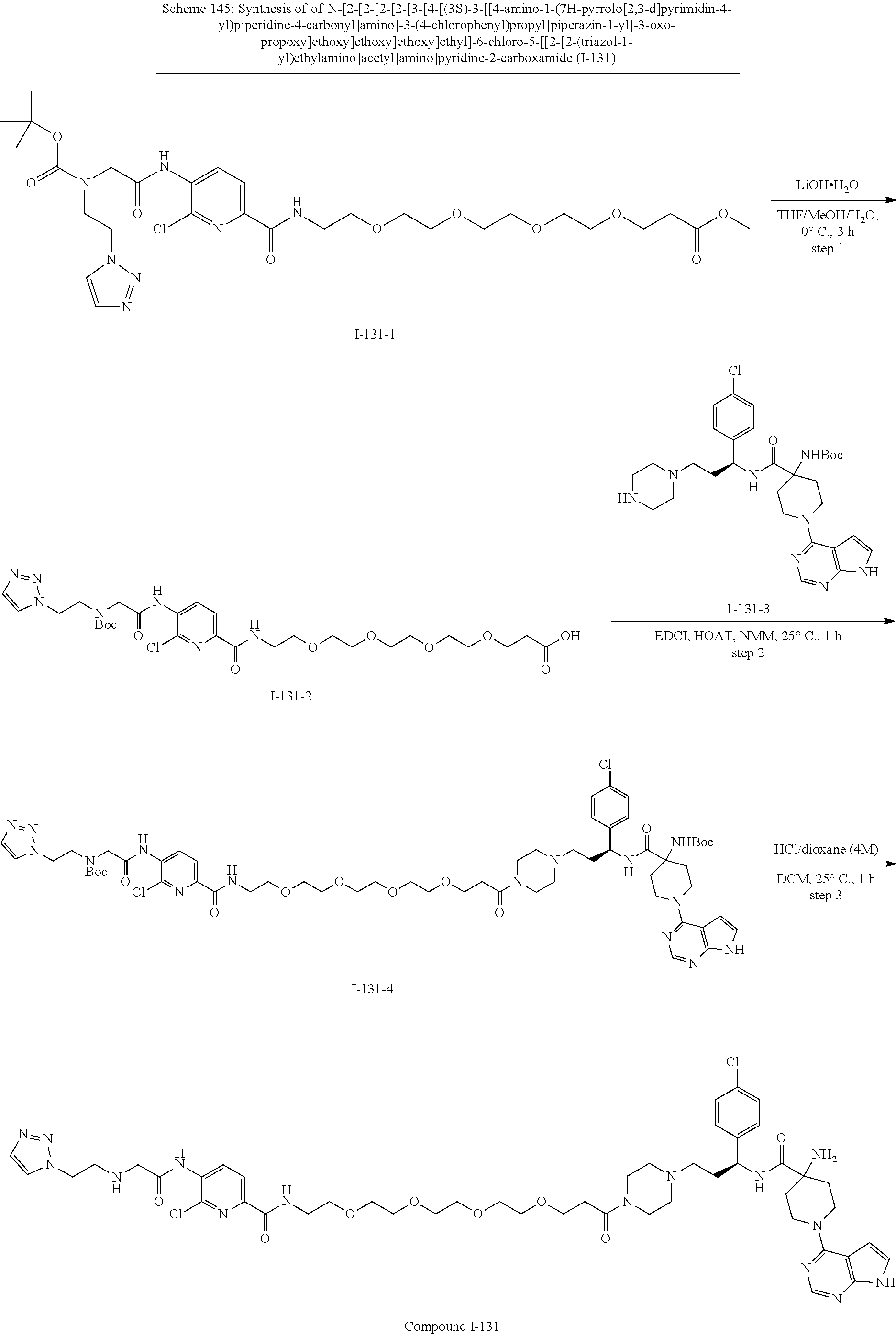

### Step 1: Synthesis of I-131-2

To a solution of I-131-1 (80 mg, 116.59 μmol, 1 eq) in THF (0.8 mL) and MeOH (0.8 mL) was added LiOH·H₂O (14.68 mg, 349.78 μmol, 3 eq) in H₂O (0.8 mL) at 0° C., and then the mixture was stirred at 0° C. for 3 h. The mixture was extracted with EA (3 mL*2). Then the aqueous phase was adjusted pH=4˜5 with 1N HCl and then extracted with EA (3 mL*5). The combined organic layers were dried over anhydrous Na₂SO₄, filtered and concentrated to give I-131-2 (76 mg, crude) as colorless oil. LCMS (Method G): R^{t}=0.312 min, [M+H]⁺=672.2.

### Step 2: Synthesis of I-131-4

To a mixture of I-131-2 (76 mg, 113.07 μmol, 1 eq), EDCI (65.03 mg, 339.22 μmol, 3 eq) and HOAt (23.09 mg, 169.61 μmol, 23.73 μL, 1.5 eq) in DCM (1 mL) was added NMM (57.19 mg, 565.37 μmol, 62.16 μL, 5 eq), the mixture was stirred at 25° C. for 5 min. Then I-131-3 (67.52 mg, 113.07 μmol, 1 eq) was added. The mixture was stirred at 25° C. for 1 hr. The mixture of (20 mg of I-131-2) was combined with another batch for work up. The combined mixture was poured into water 3 mL and extracted with DCM (3 mL*3). The combined organic layers were dried over anhydrous Na₂SO₄, filtered and concentrated to give the crude product. The crude product was purified by prep-HPLC (column: Waters Xbridge 150*25 mm*5 um; mobile phase: [water (ammonia hydroxide v/v)-ACN]; gradient: 30%-60% B over min) and the eluent was concentrated to remove MeCN and then lyophilized to afford I-131-4 (53 mg, 42.36 μmol, 63.10% yield, 100% purity) as a white solid. LCMS (Method G): R^{t}=0.631 min, [M+H]⁺=1250.8. SFC: R^{t}=1.211 min.

### Step 3: Synthesis of I-131

To a mixture of I-131-4 (53 mg, 42.36 μmol, 1 eq) in DCM (2 mL) was added HCl/dioxane (4 M, 0.5 mL), the mixture was stirred at 25° C. for 1 hr. The reaction mixture was concentrated under reduced pressure to give the crude which was combined and poured into pure water (10 mL) and then lyophilized to afford I-131 (51.79 mg, 47.62 μmol, 100% purity, HCl) as a white solid. LCMS (Method E): R^{t}=0.376 min, M+H=1052.6. SFC: R^{t}=1.440 min. ¹H NMR (400 MHZ, METHANOL-d4) δ=8.61 (d, J=8.4 Hz, 1H), 8.39 (s, 1H), 8.15 (d, J=0.8 Hz, 1H), 8.05 (d, J=8.4 Hz, 1H), 7.84 (d, J=0.8 Hz, 1H), 7.50-7.38 (m, 3H), 7.38-7.29 (m, 2H), 7.00 (d, J=4.0 Hz, 1H), 5.05-5.01 (m, 1H), 4.94-4.90 (m, 2H), 4.73-4.53 (m, 3H), 4.36-4.19 (m, 3H), 3.99-3.90 (m, 2H), 3.81-3.77 (m, 2H), 3.76-3.49 (m, 22H), 3.24-2.95 (m, 4H), 2.90-2.68 (m, 3H), 2.62-2.44 (m, 2H), 2.41-2.28 (m, 1H), 2.24 (br d, J=14.0 Hz, 1H), 2.06 (br d, J=14.8 Hz, 1H).

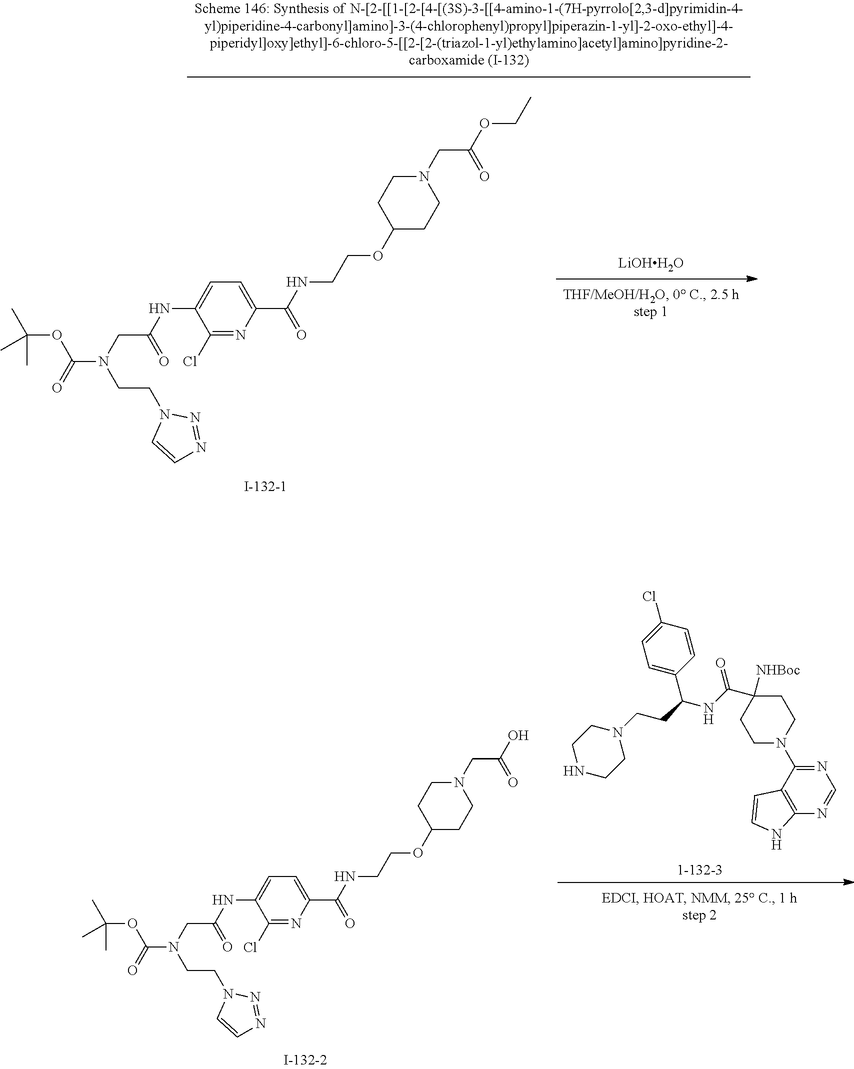

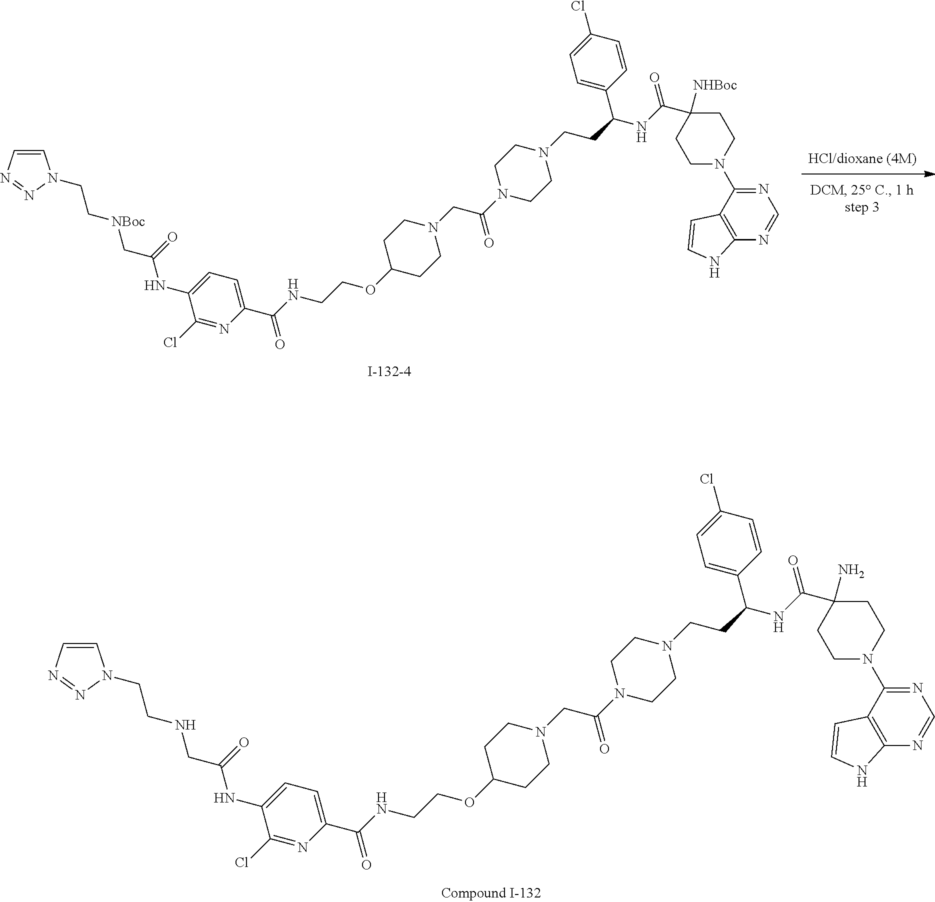 [SMILES: CC(C)(C)OC(=O)NC1(C(=O)N[C@@H](CCN2CCN(C(=O)CN3CCC(OCCNC(=O)c4ccc(NC(=O)CN(CCn5ccnn5)C(=O)OC(C)(C)C)c(Cl)n4)CC3)CC2)c2ccc(Cl)cc2)CCN(c2ncnc3[nH]ccc23)CC1 ; "HCl/dioxane (4M)" ; NC1(C(=O)N[C@@H](CCN2CCN(C(=O)CN3CCC(OCCNC(=O)c4ccc(NC(=O)CNCCn5ccnn5)c(Cl)n4)CC3)CC2)c2ccc(Cl)cc2)CCN(c2ncnc3[nH]ccc23)CC1]

### Step 1: Synthesis of I-132-2

To a solution of I-132-1 (80 mg, 125.56 μmol, 1 eq) in THF (0.8 mL) and MeOH (0.8 mL) was added LiOH·H₂O (15.81 mg, 376.69 μmol, 3 eq) in H₂O (0.8 mL) at 0° C. Then the mixture was stirred at 0° C. for 2.5 h. The mixture was adjusted to pH=4˜5 with 1N HCl and then the solution was purified by Combine flash (40 g of C18, 20-35 μm, 100 Å, Mobile phase: A for H₂O (0.1% NH₃·H₂O v/v) and B for acetonitrile; Gradient: B: 0%-20 in 20 min; Flow rate: 60 ml/min) and then lyophilized to afford I-132-2 (55 mg, 90.30 μmol, 71.92% yield, 100% purity) as colorless oil. LCMS (Method G): R^{t}=0.358 min, [M+H]⁺=609.2.

### Step 2: Synthesis of I-132-4

To a mixture of I-132-2 (55 mg, 90.30 μmol, 1 eq), EDCI (51.93 mg, 270.90 μmol, 3 eq) and HOAt (18.44 mg, 135.45 μmol, 18.95 μL, 1.5 eq) in DCM (1 mL) was added NMM (45.67 mg, 451.51 μmol, 49.64 μL, 5 eq), the mixture was stirred at 25° C. for 5 min. Then I-132-3 (53.92 mg, 90.30 μmol, 1 eq) was added. The mixture was stirred at 25° C. for 1 hr. The mixture of I-132-2 was combined for work up. The combined mixture was poured into water 3 mL and extracted with CHCl₃/isopropanol=3/1 (3 mL*3). The combined organic layers were dried over anhydrous Na₂SO₄, filtered and concentrated to give I-132-4 (110 mg, crude) as yellow oil. LCMS (Method G): R^{t}=0.626 min, [M+H]⁺=1187.8.

### Step 3: Synthesis of I-132

To a mixture of I-132-4 (90 mg, 75.74 μmol, 1 eq) in DCM (2 mL) was added HCl/dioxane (4 M, 0.8 mL, 42.25 eq), the mixture was stirred at 25° C. for 1 hr. The reaction mixture was concentrated under reduced pressure to give a residue. The residue was purified by prep-HPLC (column: Waters Xbridge 150*25 mm*5 um; mobile phase: [water (ammonia hydroxide v/v)-ACN]; gradient: 18%-48% B over min) and the eluent was concentrated to remove MeCN and then lyophilization. The product was lyophilized to afford I-132 (43.72 mg, 44.25 μmol, 58.42% yield, 100% purity) as a white solid. LCMS (Method G): R^{t}=0.522 min, M+H=987.5. SFC: R^{t}=0.916 min. ¹H NMR (400 MHz, CHLOROFORM-d) δ=9.99 (s, 1H), 9.69-9.44 (m, 1H), 8.93 (d, J=8.4 Hz, 1H), 8.74-8.58 (m, 1H), 8.37-8.22 (m, 1H), 8.14 (d, J=8.4 Hz, 1H), 8.06-7.91 (m, 1H), 7.76 (d, J=0.4 Hz, 1H), 7.64 (d, J=0.8 Hz, 1H), 7.31 (d, J=8.4 Hz, 2H), 7.19 (d, J=8.4 Hz, 2H), 7.05 (d, J=3.6 Hz, 1H), 6.55-6.46 (m, 1H), 5.11-4.93 (m, 1H), 4.63-4.47 (m, 4H), 3.69-3.53 (m, 10H), 3.50 (s, 2H), 3.42-3.33 (m, 1H), 3.32-3.09 (m, 5H), 2.82-2.67 (m, 2H), 2.48-2.12 (m, 13H), 2.07-1.85 (m, 5H), 1.57-1.49 (m, 2H).

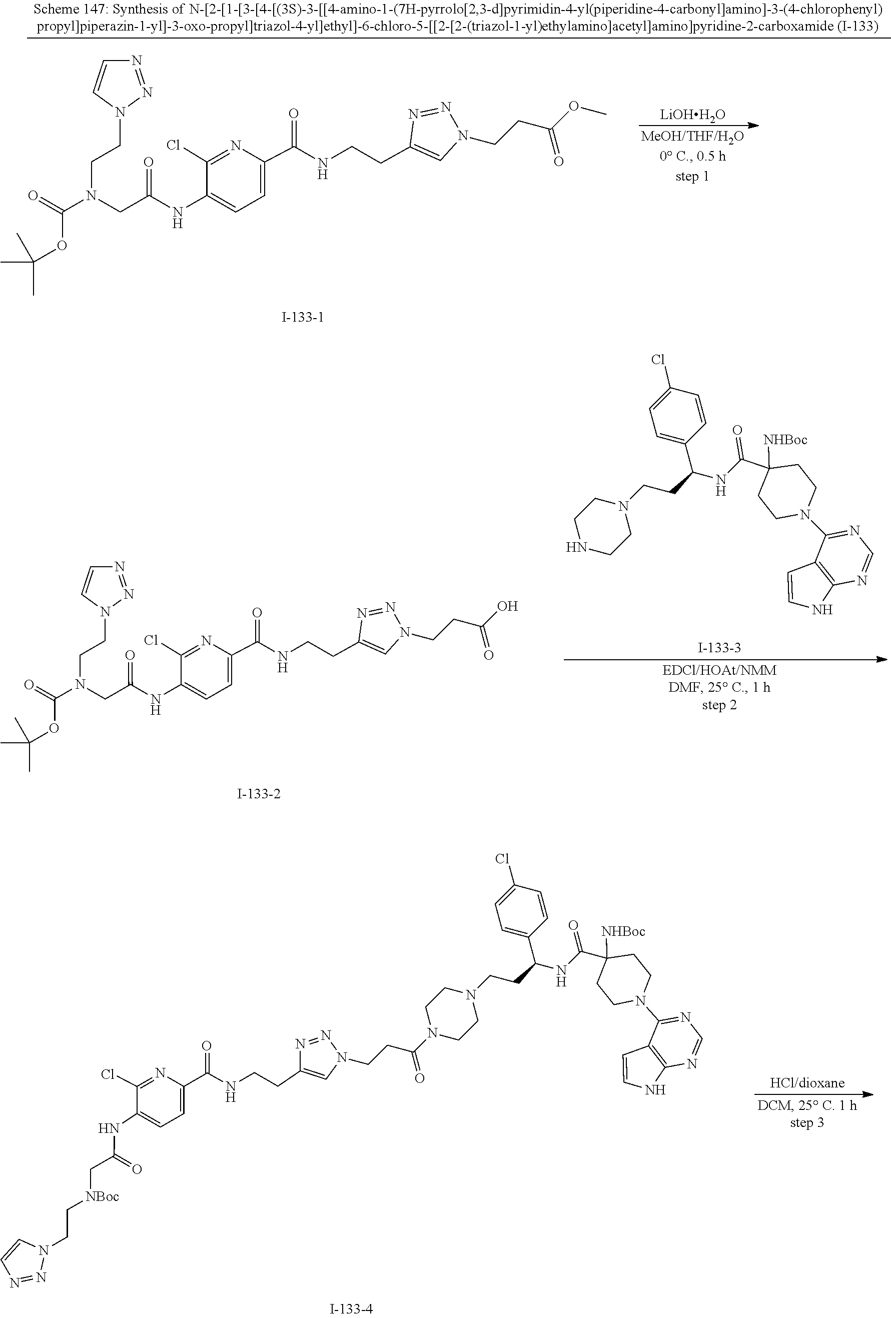

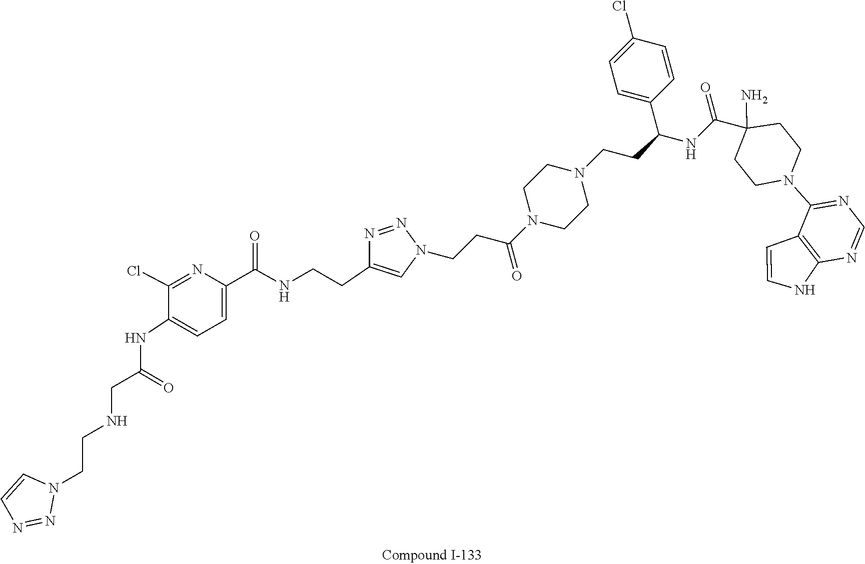 [SMILES: NC1(C(=O)N[C@@H](CCN2CCN(C(=O)CCn3cc(CCNC(=O)c4ccc(NC(=O)CNCCn5ccnn5)c(Cl)n4)nn3)CC2)c2ccc(Cl)cc2)CCN(c2ncnc3[nH]ccc23)CC1]

### Step 1: Synthesis of I-133-2

To a solution of I-133-1 (120 mg, 198.33 μmol, 1 eq) in H₂O (1 mL), MeOH (1 mL) and THF (1 mL) was added LiOH·H₂O (24.97 mg, 595.00 μmol, 3 eq). The mixture was stirred at 0° C. for 0.5 hr. The reaction was diluted saturated aqueous citric acid adjust pH=6, filtered under reduced pressure to give a residue. The crude product was purified by reverse-phase HPLC (0.1% NH₃·H₂O condition). The eluent was concentrated under reduced pressure to remove ACN, then lyophilized to afford product. I-133-2 (75 mg, 125.39 μmol, 63.22% yield, 98.809% purity) as a white solid. LCMS (Method G): R^{t}=0.298 min, [M+H]⁺=591.3.

### Step 2: Synthesis of I-133-4

To a solution of I-133-2 (60 mg, 101.52 μmol, 1 eq) and I-133-3 (60.62 mg, 101.52 μmol, 1 eq) in DMF (1 mL) was added EDCI (58.38 mg, 304.56 μmol, 3 eq), NMM (102.68 mg, 1.02 mmol, 111.61 μL, 10 eq) and HOAt (20.73 mg, 152.28 μmol, 21.30 μL, 1.5 eq). The mixture was stirred at 25° C. for 1 h. The reaction mixture was filtered give a residue. The residue was purified by reverse-phase HPLC (0.1% NH₃·H₂O condition). The eluent was concentrated under reduced pressure to remove ACN, then lyophilized to afford I-133-4 (75 mg, 64.09 μmol, 63.13% yield, 100% purity) as a white solid. LCMS (Method G): R^{t}=0.612 min, [M+H]⁺=1169.6.

### Step 3: Synthesis of I-133

The mixture of I-133-4 (65 mg, 55.55 μmol, 1 eq) in DCM (1 mL) and HCl/dioxane (0.2 mL) was stirred at 25° C. for 1 h. The reaction was combined with another batch, filtered and concentrated under reduced pressure to give a residue. The residue was lyophilized to afford I-133 (71.75 mg, 57.89 μmol, 95.9% purity, HCl) as a white solid. LCMS (Method G): R^{t}=0.495 min, [M+H]⁺=969.4. SFC: R^{t}=1.851 min. ¹H NMR (400 MHZ, METHANOL-d4) δ=8.59 (d, J=8.4 Hz, 1H), 8.39 (s, 1H), 8.20 (d, J=9.6 Hz, 2H), 8.02 (d, J=8.4 Hz, 1H), 7.87 (s, 1H), 7.48-7.39 (m, 3H), 7.33 (d, J=8.4 Hz, 2H), 7.01 (d, J=3.6 Hz, 1H), 5.07-5.03 (m, 1H), 4.96-4.93 (m, 2H), 4.78-4.75 (m, 2H), 4.67-4.47 (m, 3H), 4.29 (s, 2H), 4.18-4.06 (m, 1H), 4.02-3.94 (m, 2H), 3.81-3.78 (m, 2H), 3.74-3.71 (m, 2H), 3.68-3.57 (m, 2H), 3.55-3.46 (m, 1H), 3.41-3.33 (m, 1H), 3.28-3.17 (m, 3H), 3.14-3.00 (m, 5H), 2.90-2.81 (m, 1H), 2.80-2.69 (m, 1H), 2.61-2.46 (m, 1H), 2.40-2.21 (m, 2H), 2.08 (d, J=14.6 Hz, 1H).

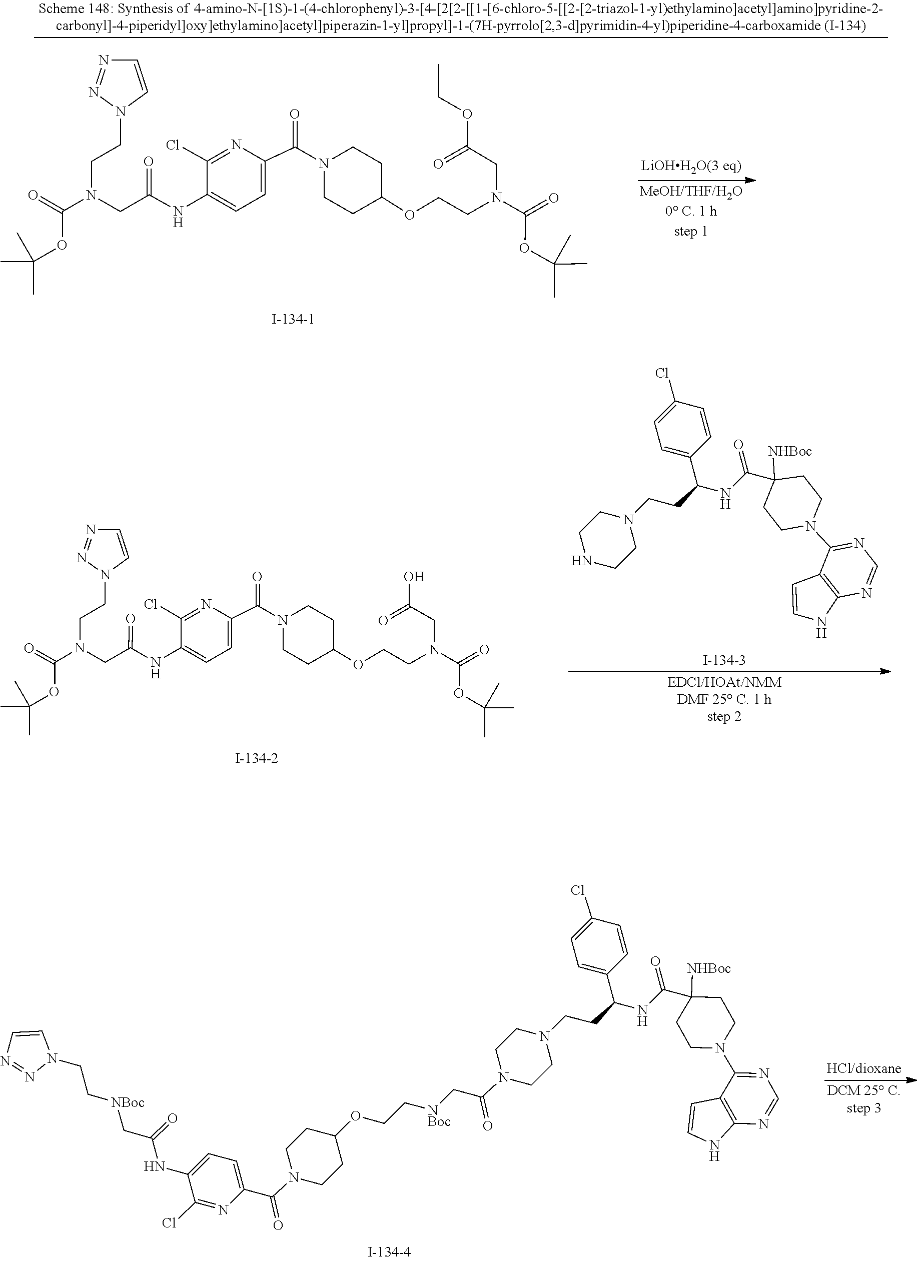

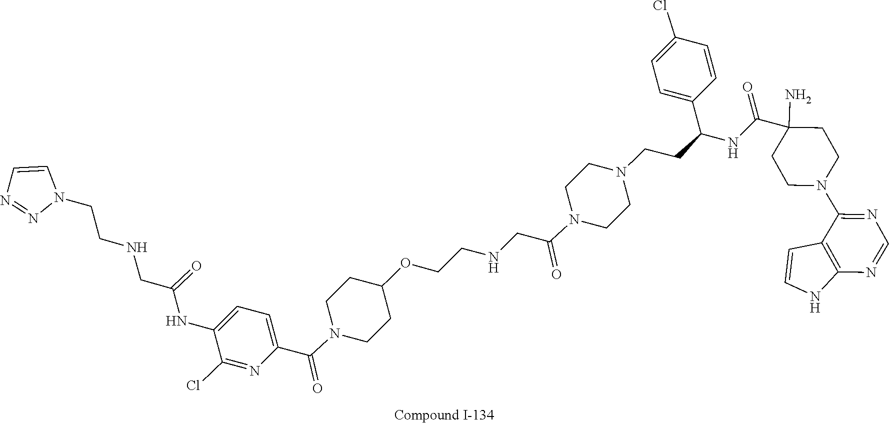 [SMILES: NC1(C(=O)N[C@@H](CCN2CCN(C(=O)CNCCOC3CCN(C(=O)c4ccc(NC(=O)CNCCn5ccnn5)c(Cl)n4)CC3)CC2)c2ccc(Cl)cc2)CCN(c2ncnc3[nH]ccc23)CC1]

### Step 1: Synthesis of I-134-2

To a solution of I-134-1 (120 mg, 162.77 μmol, 1 eq) in MeOH (1 mL), THF (1 mL) and H₂O (1 mL) was added LiOH·H₂O (20.49 mg, 488.31 μmol, 3 eq). The mixture was stirred at 0° C. for 1 hr. The reaction was diluted saturated aqueous citric acid adjust pH=6, filtered under reduced pressure to give a residue. The residue was purified by reverse-phase HPLC (0.1% NH₃·H₂O condition). The eluent was concentrated under reduced pressure to remove ACN, then lyophilized to afford I-134-2 (75 mg, 102.06 μmol, 62.70% yield, 96.505% purity) as a white solid. LCMS (Method G): R^{t}=0.356 min, [M+H]⁺=709.4.

### Step 2: Synthesis of I-134-4

To a solution of I-134-3 (60.63 mg, 101.52 μmol, 1.2 eq) and I-134-2 (60 mg, 84.60 μmol, 1 eq) in DMF (1 mL) was added EDCI (48.66 mg, 253.81 μmol, 3 eq), NMM (85.58 mg, 846.04 μmol, 93.02 μL, 10 eq) and HOAt (17.27 mg, 126.91 μmol, 17.75 μL, 1.5 eq). The mixture was stirred at 25° C. for 1 hr. The reaction mixture was filtered give a residue. The residue was purified by reverse-phase HPLC (0.1% NH₃·H₂O condition). The eluent was concentrated under reduced pressure to remove ACN, then lyophilized to afford I-134-4 (75 mg, 58.22 μmol, 68.81% yield, 100% purity) as a white solid. LCMS (Method G): R^{t}=0.681 min, [M+H]⁺=1287.7.

### Step 3: Synthesis of I-134

The mixture of I-134-4 (65 mg, 50.45 μmol, 1 eq) in HCl/dioxane (0.2 mL) and DCM (1 mL) was stirred at 25° C. for 1 hr. The reaction was combined with another batch, filtered and concentrated under reduced pressure to give a residue. The residue was lyophilized to afford I-134 (69 mg, 55.50 μmol, 100% purity, HCl) as an orange solid. LCMS (Method G): R^{t}=0.497 min, [M+H]⁺=989.5. SFC: R^{t}=1.663 min. ¹H NMR (400 MHZ, METHANOL-d4) δ=8.55 (d, J=8.4 Hz, 1H), 8.39 (s, 1H), 8.20 (s, 1H), 7.91 (s, 1H), 7.63 (d, J=8.4 Hz, 1H), 7.49-7.40 (m, 3H), 7.35 (d, J=8.4 Hz, 2H), 7.02 (d, J=3.6 Hz, 1H), 5.07-5.03 (m, 1H), 4.95-4.90 (m, 4H), 4.69-4.54 (m, 3H), 4.36-4.23 (m, 3H), 4.20-4.10 (m, 1H), 4.08-3.90 (m, 4H), 3.84-3.82 (m, 2H), 3.80-3.77 (m, 3H), 3.74-3.52 (m, 5H), 3.40-3.33 (m, 3H), 3.28-3.10 (m, 3H), 2.93-2.80 (m, 1H), 2.78-2.66 (m, 1H), 2.64-2.45 (m, 1H), 2.41-2.20 (m, 2H), 2.13-1.91 (m, 3H), 1.85-1.63 (m, 2H).

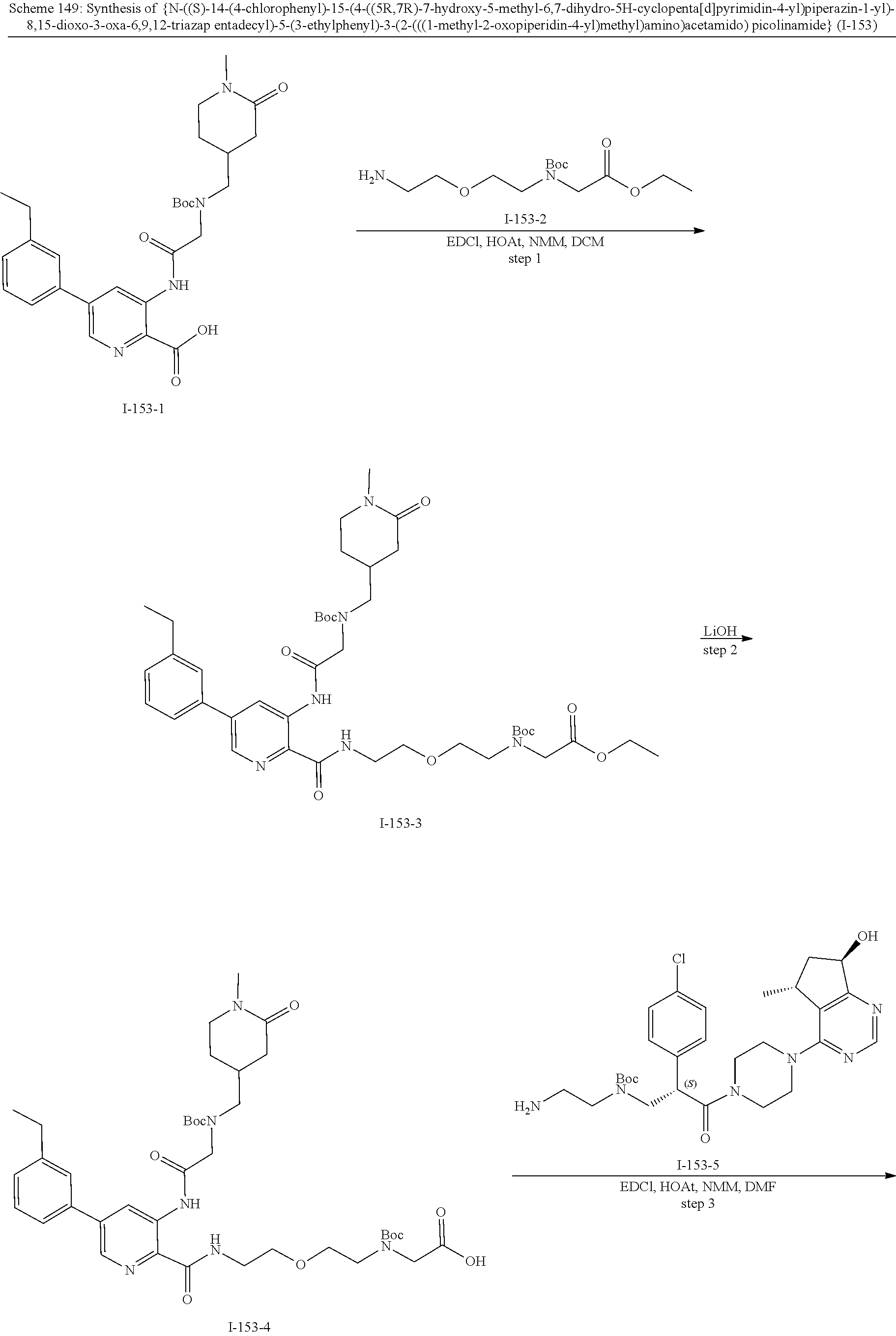

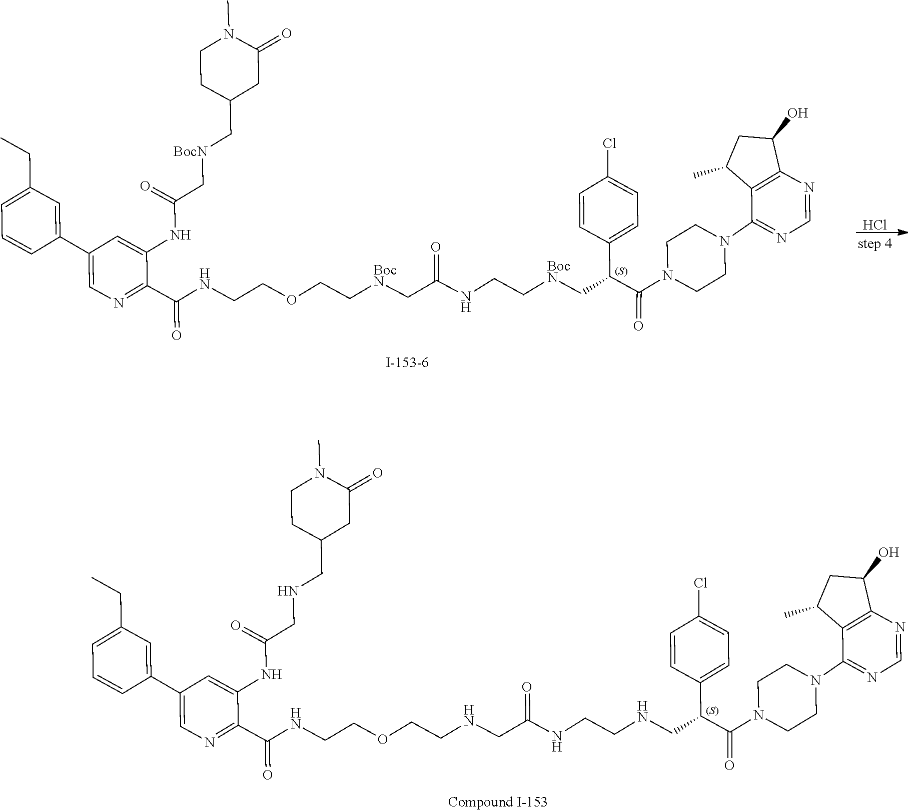

### Step 1: Synthesis of I-153-3

To a solution of I-153-1 (0.1 g, 190.62 μmol, 1 eq) in DCM (1 mL) was added I-153-2 (66.42 mg, 228.74 μmol, 1.2 eq) HOAt (25.95 mg, 190.62 μmol, 26.67 μL, 1 eq) and EDCI (36.54 mg, 190.62 μmol, 1 eq) NMM (96.40 mg, 953.09 μmol, 104.79 μL, 5 eq). The mixture was stirred at 25° C. for 1 hr. showed a major peak of desired MW. The reaction mixture was added H₂O (2 mL) and extracted with DCM (3 mL*3) and washed with brine (2 mL*4). The organic layer was dried with anhydrous Na₂SO₄, filtered and concentrated under vacuum to get the residue. The residue was purified by flash silica gel chromatography (ISCO®; 20 g SepaFlash® Silica Flash Column, Eluent of 0˜10% DCM/MeOH ether gradient @ XX mL/min) to give I-153-3 (0.12 g, 150.57 μmol, 78.99% yield) as a yellow oil. LCMS (Method G): R^{t}=0.681 min, [M+H]⁺=797.5.

### Step 2: Synthesis of I-153-4

To a solution of I-153-3 (0.12 g, 150.57 μmol, 1 eq) in EtOH (0.5 mL)/THF (0.5 mL)/H2O (0.5 mL) was added LiOH·H₂O (31.59 mg, 752.87 μmol, 5 eq). The mixture was stirred at 25° C. for 1.5 hr. The reaction mixture was adjusted to pH=4 with 1N HCl, Precipitation of solids. Then filtrated, the filter cake was concentrated to get I-153-4 (0.11 g, crude) as a white solid. LCMS (Method E): R^{t}=0.613 min, [M+H]⁺=769.5.

### Step 3: Synthesis of I-153-6

To a solution of I-153-4 (0.08 g, 143.09 μmol, 1 eq) in DCM (1 mL) was added I-153-5 (110.02 mg, 143.09 μmol, 1 eq), HOAt (19.48 mg, 143.09 μmol, 20.02 μL, 1 eq) and EDCI (27.43 mg, 143.09 μmol, 1 eq), NMM (72.36 mg, 715.44 μmol, 78.66 μL, 5 eq). The mixture was stirred at 25° C. for 1 hr. showed 35% of desired MW. The reaction mixture was added H₂O (2 mL) and extracted with DCM (3 mL*3) and washed with brine (2 mL*4). The organic layer was dried with anhydrous Na₂SO₄, filtered and concentrated under vacuum to get the residue. The residue was purified by prep-HPLC (FA condition) and extracted with DCM (20 mL*3) then concentrated to get the residue. The residue was purified by prep-HPLC (neutral condition) column: Waters Xbridge 150*25 mm*5 um; mobile phase: [water (NH4HCO3)-ACN]; gradient: 58%-88% B over 9 min and concentrated to get I-153-6 (0.015 g, crude) as a yellow oil. LCMS (Method E): R^{t}=0.807 min, [M+H]⁺=1309.9.

### Step 4: Synthesis of I-153

To a solution of I-153-6 (15 mg, 11.45 μmol, 1 eq) in dioxane (0.2 mL) was added HCl/dioxane (4 M, 0.2 mL, 69.87 eq). The mixture was stirred at 25° C. for 1 h. The mixture was concentrated under vacuum to remove dioxane to give a yellow oil. The yellow oil was diluted with H₂O (20 mL) and freeze-dried to give I-153 (12.88 mg, crude, HCl) as a yellow solid. LCMS: R^{t}=0.656 min, [M+H]⁺=1009.7. SFC: R^{t}=0.877 min. ¹H NMR (400 MHZ, DMSO-d₆) δ=9.10 (d, J=1.6 Hz, 1H), 8.80-8.66 (m, 2H), 7.52 (br s, 2H), 7.50-7.42 (m, 3H), 7.39-7.33 (m, 3H), 5.24 (m, 1H), 4.70 (m, 1H), 4.19-4.02 (m, 1H), 3.89 (m, 1H), 3.79 (s, 3H), 3.61 (m, 3H), 3.58-3.52 (m, 5H), 3.49-3.40 (m, 3H), 3.38-3.23 (m, 4H), 3.18-3.11 (m, 3H), 3.06 (, 2H), 3.01-2.94 (m, 2H), 2.79 (s, 3H), 2.71-2.66 (m, 2H), 2.45 (m, 1H), 2.43-2.38 (m, 1H), 2.35-2.20 (m, 2H), 2.16-1.96 (m, 5H), 1.54-1.47 (m, 1H), 1.21 (m, 4H), 1.05 (m, 3H).

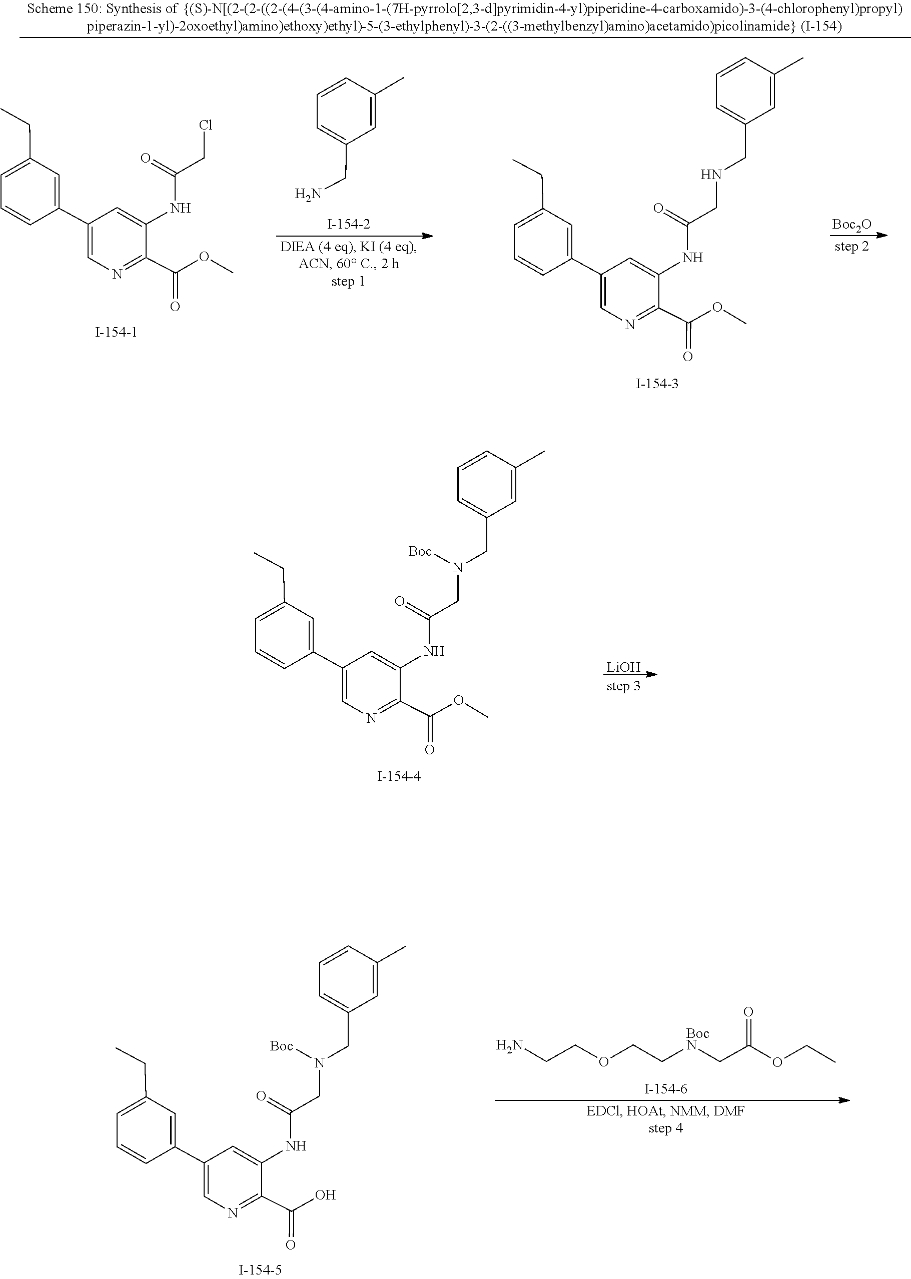

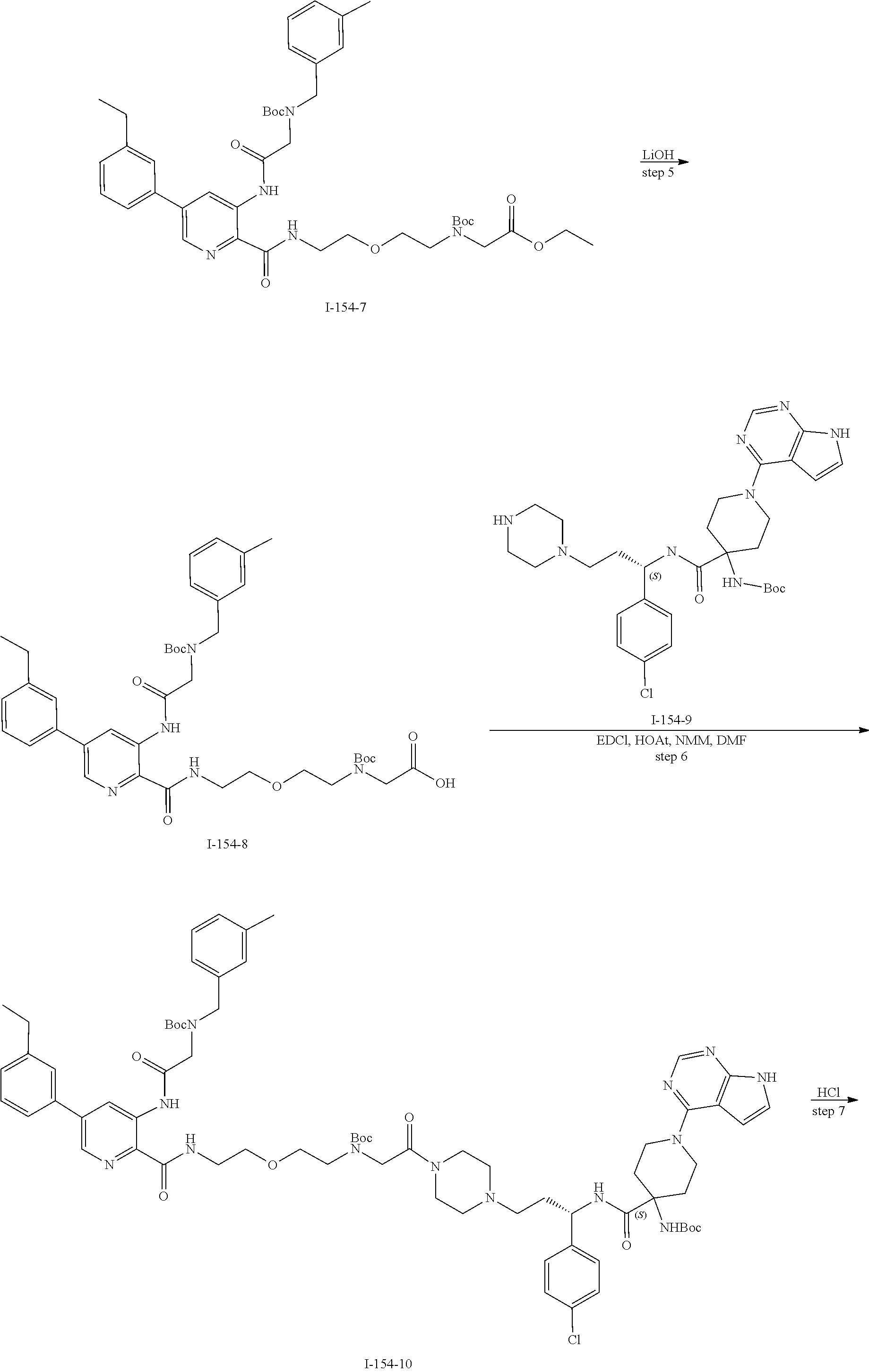

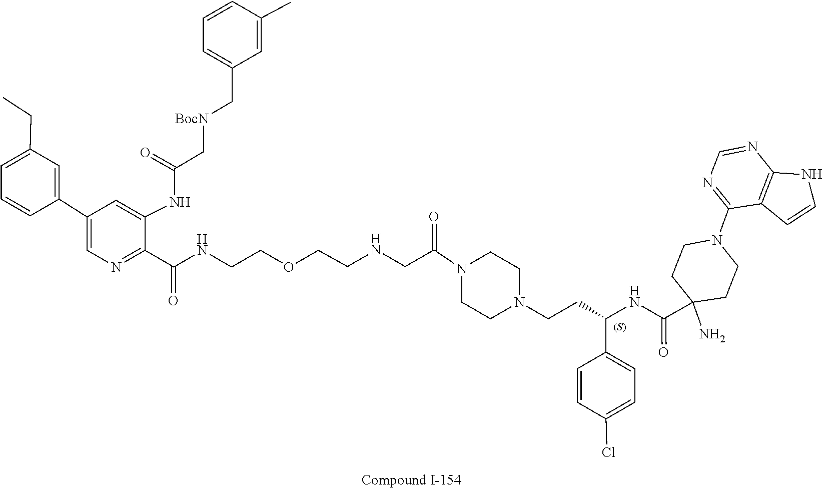

### Step 1: Synthesis of I-154-3

To a solution of I-154-1 (1 g, 3.00 mmol, 1 eq) in ACN (10 mL) was added I-154-2 (436.97 mg, 3.61 mmol, 452.35 μL, 1.2 eq), DIEA (1.55 g, 12.02 mmol, 2.09 mL, 4 eq) and KI (2.00 g, 12.02 mmol, 4 eq). The mixture was stirred at 60° C. for 2 hr. showed a major peak of desired MW. The reaction mixture was used for next step and no work. LCMS (Method G): R^{t}=0.754 min, [M+H]⁺=418.3.

### Step 2: Synthesis of I-154-4

To a solution of I-154-3 (1.25 g, 2.99 mmol, 1 eq) in ACN (10 mL) was added Boc2O (980.15 mg, 4.49 mmol, 1.03 mL, 1.5 eq). The mixture was stirred at 40° C. for 1 hr. To the reaction was added H2O (5 mL) and extracted with EA (10 mL*3) and washed with brine (5 mL*4). The organic layer was dried with anhydrous Na₂SO₄, filtered and concentrated under vacuum to get the residue. The residue was purified by flash silica gel chromatography (ISCO®; X g SepaFlash® Silica Flash Column, Eluent of 0˜50% Ethyl acetate/Petroleum ether gradient @ XX mL/min) to give I-154-4 (1.5 g, 2.77 mmol, 92.53% yield, 95.6% purity) as a yellow oil. LCMS (Method E): R^{t}=0.709 min, [M+H]⁺=518.4.

### Step 3: Synthesis of I-154-5

To a solution of I-154-4 (1.5 g, 2.90 mmol, 1 eq) in MeOH (5 mL)/THF (5 mL)/H2O (5 mL) was added LiOH·H₂O (364.82 mg, 8.69 mmol, 3 eq). The mixture was stirred at 25° C. for 2 hr. The reaction mixture was adjusted to PH=7 with 1N HCl, Precipitation of solids. Then filtrated, the filter cake was concentrated to get I-154-5 (1.2 g. 2.38 mmol, 82.23% yield) as a white solid. LCMS (Method E): R^{t}=0.665 min, [M+H]⁺=448.4. ¹H NMR (400 MHZ, DMSO-d₆) δ=13.15-12.80 (m, 1H), 9.21 (br d, J=4.0 Hz, 1H), 8.58 (s, 1H), 7.57-7.49 (m, 2H), 7.48-7.42 (m, 1H), 7.32 (d, J=7.5 Hz, 1H), 7.25-7.17 (m, 1H), 7.14-7.04 (m, 3H), 4.53 (s, 2H), 4.01-3.90 (m, 3H), 2.70 (q, J=7.5 Hz, 2H), 2.27 (s, 3H), 1.44-1.31 (m, 9H), 1.23 (t, J=7.6 Hz, 3H).

### Step 4: Synthesis of I-154-7

To a solution of I-154-5 (105.08 mg, 361.89 μmol, 1 eq) in DCM (1 mL) was I-154-6 (200.47 mg, 398.08 μmol, 1.1 eq), HOAt (49.26 mg, 361.89 μmol, 50.62 μL, 1 eq) and EDCI (69.37 mg, 361.89 μmol, 1 eq), NMM (183.02 mg, 1.81 mmol, 198.93 μL, 5 eq). The mixture was stirred at 25° C. for 12 hr. The reaction mixture was added H2O (2 mL) and extracted with DCM (3 mL*3) and washed with brine (2 mL*4). The organic layer was dried with anhydrous Na₂SO₄, filtered and concentrated under vacuum to get the residue. The residue was purified by flash silica gel chromatography (ISCO®; 20 g SepaFlash® Silica Flash Column, Eluent of 0˜60% Ethyl acetate/Petroleum ether gradient @ XX mL/min) to give I-154-7 (230 mg, 296.42 μmol, 81.91% yield) as a yellow oil. LCMS (Method E): R^{t}=0.775 min, [M+H]⁺=776.5.

### Step 5: Synthesis of I-154-8

To a solution of ethyl I-154-7 (0.23 g, 296.42 μmol, 1 eq) in EtOH (1 mL)/THF (1 mL)/H2O (1 mL) was added LiOH·H₂O (62.19 mg, 1.48 mmol, 5 eq). The mixture was stirred at 25° C. for 1.5 hr. The reaction mixture was adjusted to PH=4 with 1N HCl, Precipitation of solids. Then filtrated, the filter cake was concentrated to get I-154-8 (0.2 g, crude) as a white solid. LCMS (Method E): R^{t}=0.714 min, [M+H]⁺=748.5.

### Step 6: Synthesis of I-154-10

To a solution of I-154-8 (0.15 g, 251.19 μmol, 1 eq) in DMF (1 mL) was added I-154-10 (187.86 mg, 251.19 μmol, 1 eq), HOAt (34.19 mg, 251.19 μmol, 35.14 μL, 1 eq) and EDCI (48.15 mg, 251.19 umol, 1 eq), NMM (127.04 mg, 1.26 mmol, 138.08 μL, 5 eq). The mixture was stirred at 25° C. for 1 hr. The reaction mixture was added H2O (2 mL) and extracted with DCM (3 mL*3) and washed with brine (2 mL*4). The organic layer was dried with anhydrous Na₂SO₄, filtered and concentrated under vacuum to get the residue. The residue was purified by prep-HPLC (FA condition) and extracted with DCM (20 mL*3) then concentrated to get the residue. The residue was purified by prep-HPLC (neutral condition) column: Waters Xbridge 150*25 mm*5 um; mobile phase: [water (NH4HCO3)-ACN]; gradient: 75%-100% B over 9 min) to give I-154-10 (0.05 g, crude) as a white solid. LCMS (Method E): R^{t}=0.602 min, [M+H]⁺=1326.9.

### Step 7: Synthesis of I-154

To a solution of I-154-10 (50 mg, 37.68 μmol, 1 eq) in dioxane (0.5 mL) was added HCl/dioxane (4 M, 0.5 mL, 53.08 eq). The mixture was stirred at 25° C. for 1 h. The mixture was concentrated under vacuum to remove dioxane to give a yellow oil. The yellow oil was diluted with H₂O (20 ml) and freeze-dried to give I-154 (42.73 mg, crude, HCl) as a yellow solid. LCMS (Method a): R^{t}=0.789 min, [M+H]⁺=1026.7. SFC: R^{t}=3.778 min. ¹H NMR (400 MHZ, DMSO-d₆) δ=9.04 (d, J=1.6 Hz, 1H), 8.67 (d, J=2.0 Hz, 1H), 8.36 (s, 1H), 7.53-7.51 (m, 2H), 7.48 (d, J=7.6 Hz, 1H), 7.44 (d, J=3.6 Hz, 1H), 7.39 (s, 4H), 7.36 (s, 1H), 7.34-7.31 (m, 3H), 7.29-7.20 (m, 1H), 6.96 (d, J=3.6 Hz, 1H), 4.92 (m, 1H), 4.36-4.30 (m, 2H), 4.19-4.09 (m, 6H), 3.91 (m, 2H), 3.65-3.59 (m, 3H), 3.54 (m, 4H), 3.22-3.08 (m, 6H), 2.68 (d, J=7.6 Hz, 2H), 2.60-2.54 (m, 1H), 2.30 (s, 5H), 2.25-2.04 (m, 3H), 2.03-1.88 (m, 2H), 1.25-1.16 (m, 6H).

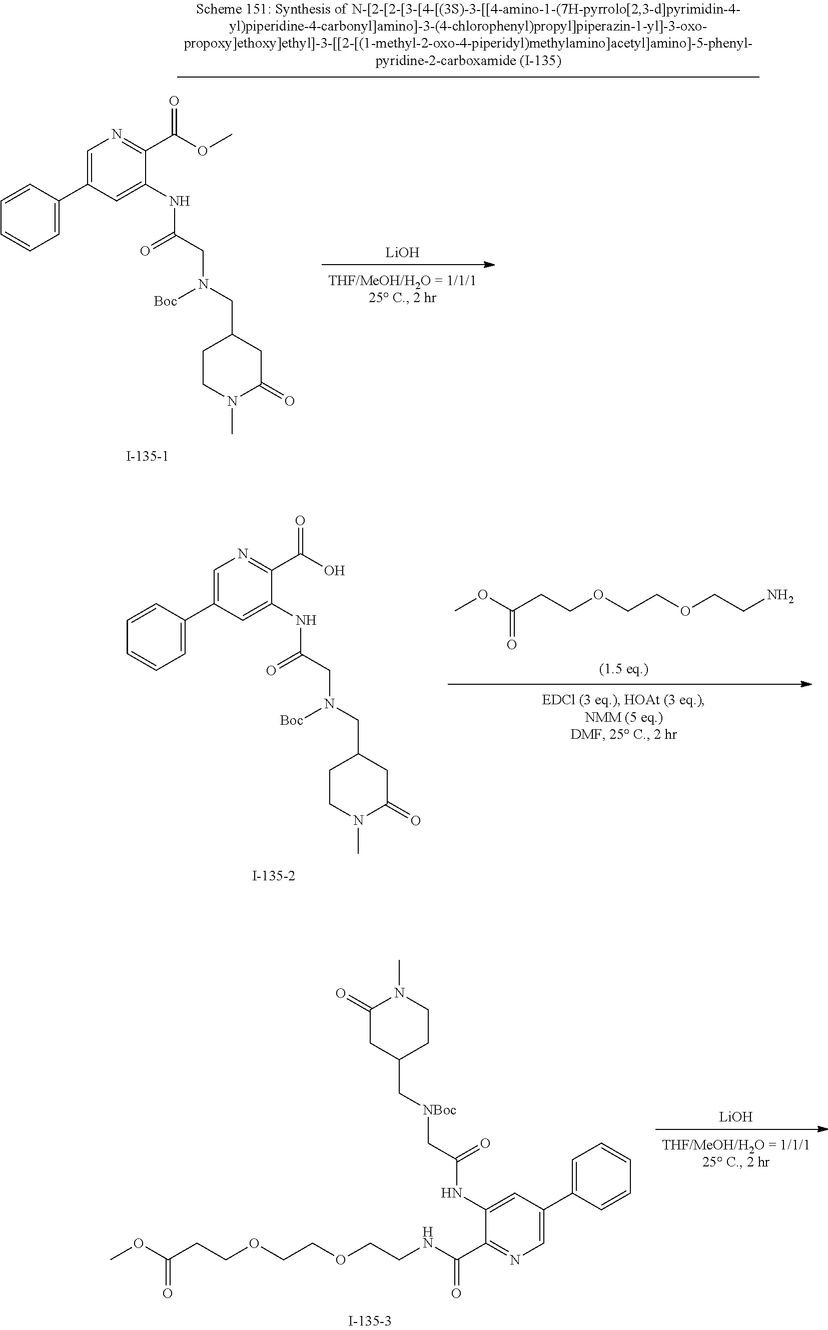

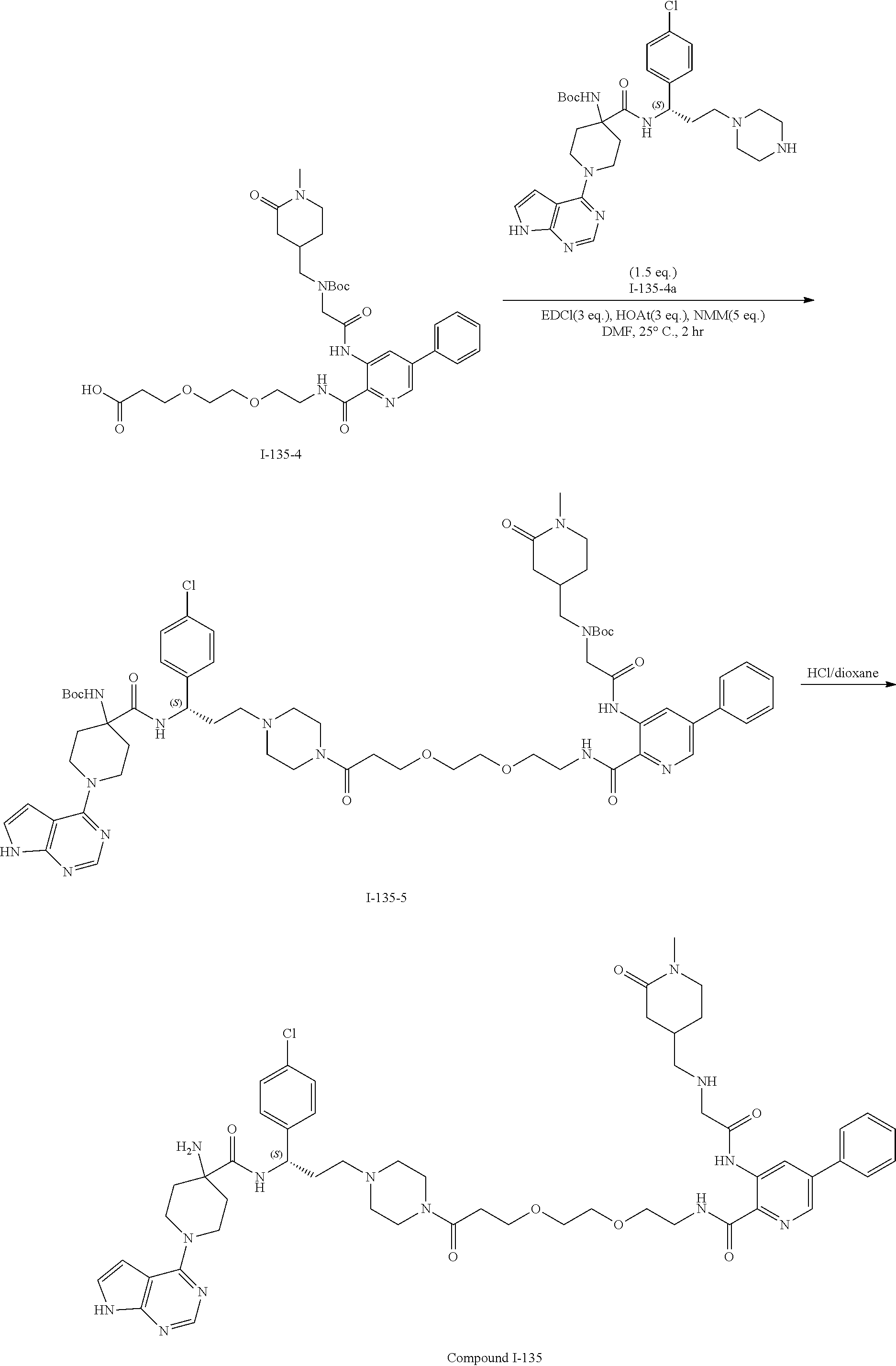

### Step 1: Synthesis of I-135-2

To a solution of I-135-1 (200 mg, 391.71 μmol, 1 eq) in THF (1 mL), MeOH (1 mL), H2O (1 mL) was added LiOH·H₂O (164.38 mg, 3.92 mmol, 10 eq). The mixture was stirred at 25° C. for 1 hr. The reaction solution was acidified with citric acid to pH=5-6. The reaction mixture was washed with water (10 mL) and extracted with EA (10 mL*2). The combined organic phase was washed with brine (20 mL*1), dried with anhydrous Na₂SO₄, filtered, and concentrated in vacuum to give I-135-2 (200 mg, 362.50 μmol, 92.54% yield, 90% purity) as a brown solid. LCMS: R^{t}=0.372 min, [M+H]⁺=495.2.

### Step 2: Synthesis of I-135-3

To a solution of I-135-2 (200 mg, 402.78 μmol, 1 eq), methyl 3-[2-(2-aminoethoxy) ethoxy]propanoate (115.53 mg, 604.16 μmol, 1.5 eq) in DMF (2 mL) was added EDCI (231.64 mg, 1.21 mmol, 3 eq), NMM (203.70 mg, 2.01 mmol, 221.41 μL, 5 eq) and HOAt (164.47 mg, 1.21 mmol, 169.03 μL, 3 eq). The mixture was stirred at 25° C. for 2 hr. The reaction mixture was washed with water (20 mL) and extracted with EA (10 mL*2). The combined organic phase was washed with brine (20 mL*1), dried with anhydrous Na₂SO₄, filtered, and concentrated in vacuum to give a yellow oil. The mixture was purified by reverse-phase HPLC (0.1% FA condition) to give I-135-3 (150 mg, 199.32 μmol, 49.49% yield, 89% purity) as a yellow oil. LCMS (Method G): R^{t}=0.653 min, [M+H]⁺=670.5.

### Step 3: Synthesis of I-135-4

To a solution of I-135-3 (150 mg, 223.96 μmol, 1 eq) in THF (1 mL), MeOH (1 mL), H₂O (1 mL) was added LiOH·H₂O (93.98 mg, 2.24 mmol, 10 eq). The mixture was stirred at 25° C. for 1 hr. The reaction solution was acidified with HCl (1 M) to pH=5-6. The reaction mixture was washed with water (10 mL) and extracted with DCM (6 mL*2). The combined organic phase was washed with brine (10 mL*1), dried with anhydrous Na₂SO₄, filtered and concentrated in vacuum to give I-135-4 (140 mg, 180.83 μmol, 80.74% yield, 84.7% purity) as a brown solid. LCMS (Method E): R^{t}=0.525 min, [M+H]⁺=656.4.

### Step 4: Synthesis of I-135-5

To a solution of I-135-4 (70 mg, 106.75 μmol, 1 eq), tert-butyl N-[4-[[(1S)-1-(4-chlorophenyl)-3-piperazin-1-yl-propyl]carbamoyl]-1-(7H-pyrrolo[2,3-d]pyrimidin-4-yl)-4-piperidyl]carbamate (76.49 mg, 128.10 μmol, 1.2 eq) in DMF (1 mL) was added EDCI (61.39 mg, 320.25 μmol, 3 eq), NMM (53.99 mg, 533.75 μmol, 58.68 μL, 5 eq) and HOAt (43.59 mg, 320.25 μmol, 44.80 μL, 3 eq). The mixture was stirred at 25° C. for 15 hr. The reaction mixture was washed with water (10 mL) and extracted with DCM (4 mL*2). The combined organic phase was washed with brine (10 mL*1), dried with anhydrous Na₂SO₄, filtered and concentrated in vacuum to give I-135-5 (140 mg, 104.87 μmol, 98.24% yield) as a yellow oil. LCMS (Method E): R^{t}=0.488 min, [M+H−100]⁺=1234.6.

### Step 5: Synthesis of I-135

To a solution of I-135-5 (140 mg, 104.87 μmol, 1 eq) in dioxane (0.5 mL) was added HCl/dioxane (4 M, 1.5 mL, 57.21 eq). The mixture was stirred at 25° C. for 1 hr. The reaction mixture was concentrated under reduced pressure to give a brown foam. The brown solid was purified by prep-HPLC (column: Phenomenex luna C18 150*25 mm*10 um; mobile phase: [water (HCl)-ACN]; gradient: 5%-35% B over 10 min) to obtain a solution and then freeze-dried to give I-135 (17.93 mg, 16.98 μmol, 16.19% yield, 98% purity) as a off-white solid. LCMS (Method E): R^{t}=0.407 min, [M+H]⁺=1034.6. ¹H NMR (400 MHZ, METHANOL-d4) δ=9.23 (d, J=1.6 Hz, 1H), 8.64 (d, J=1.2 Hz, 1H), 8.33 (br s, 1H), 7.70 (br d, J=7.6 Hz, 2H), 7.55-7.41 (m, 5H), 7.36-7.31 (m, 3H), 6.91 (br d, J=3.6 Hz, 1H), 5.04 (m, 2H), 4.56 (br d, J=14.4 Hz, 2H), 4.27 (s, 2H), 3.97-3.86 (m, 3H), 3.76 (br t, J=6.4 Hz, 3H), 3.69-3.59 (m, 8H), 3.54-3.36 (m, 5H), 3.22-3.13 (m, 3H), 2.94 (s, 3H), 2.83-2.62 (m, 5H), 2.61-2.30 (m, 4H), 2.30-1.95 (m, 5H), 1.74-1.61 (m, 1H).

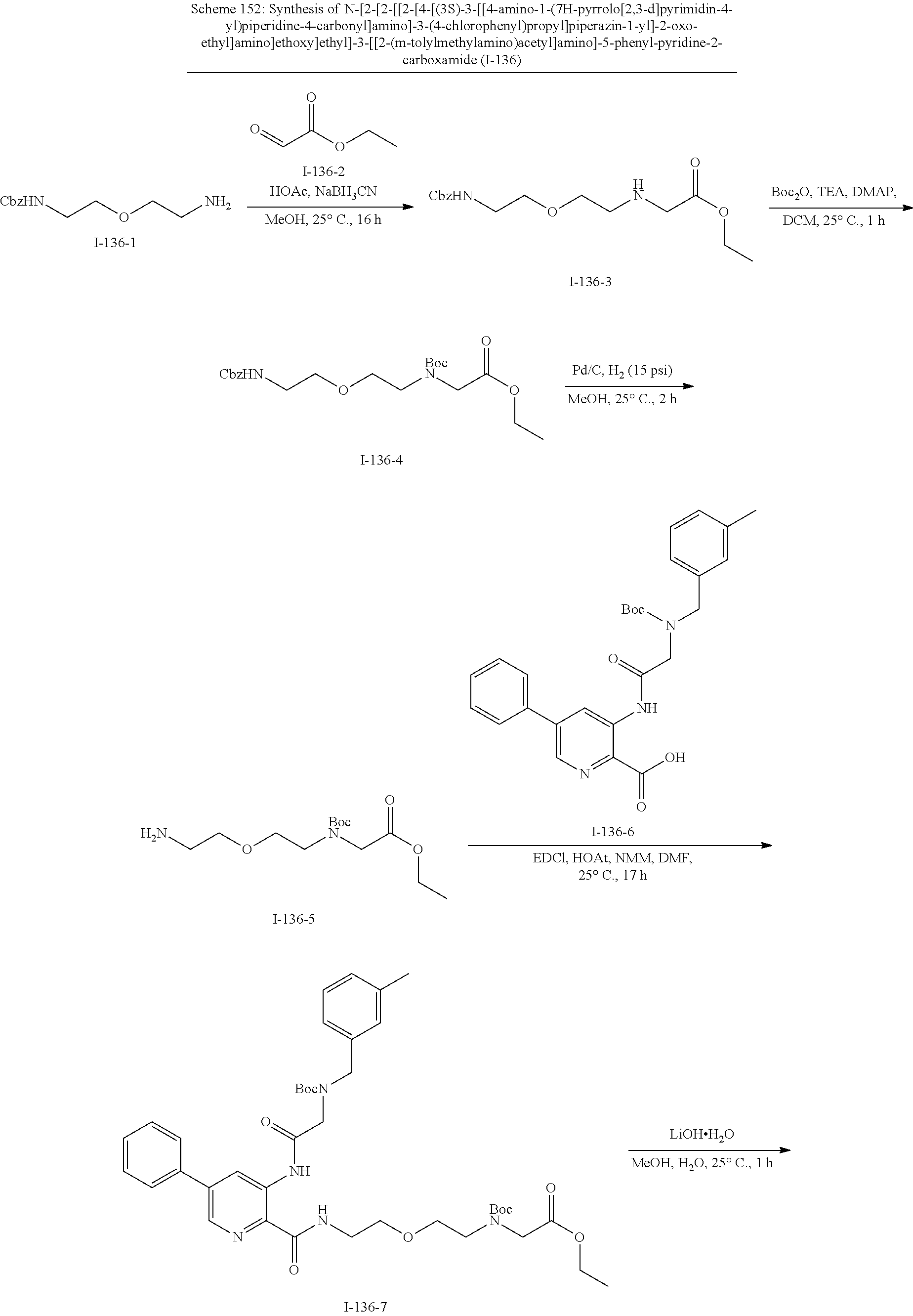

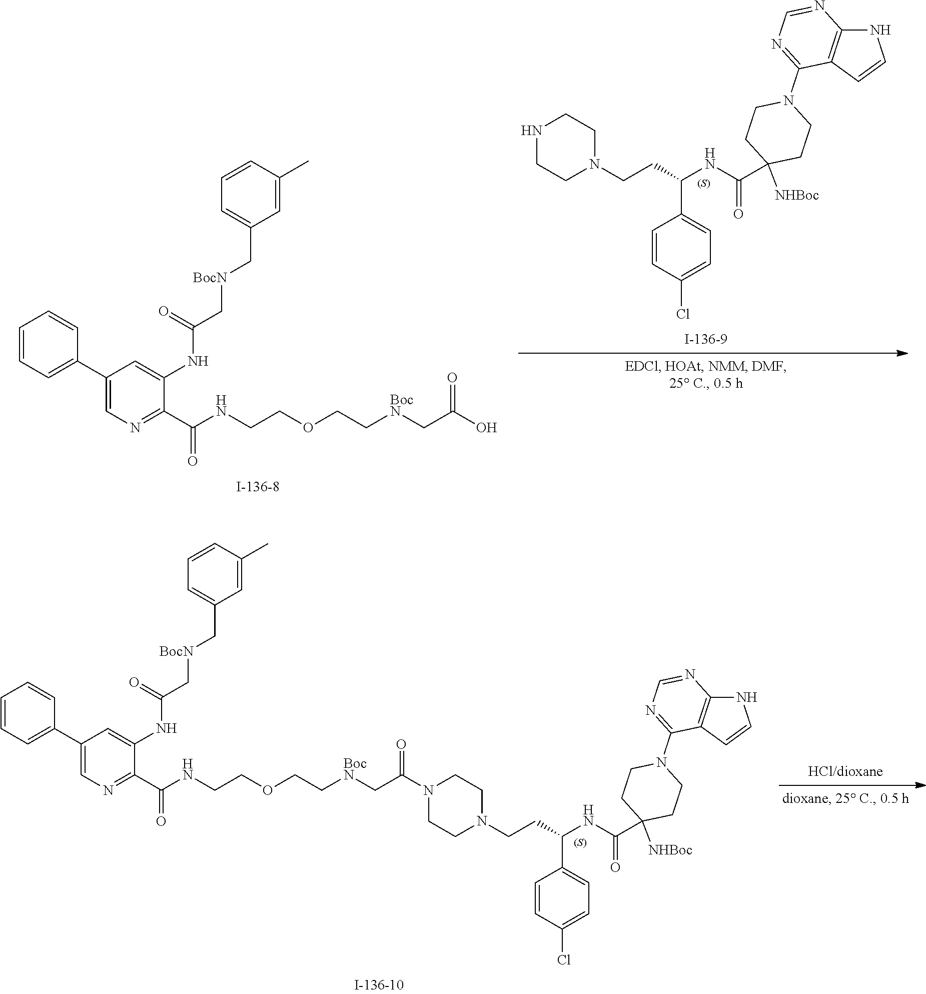

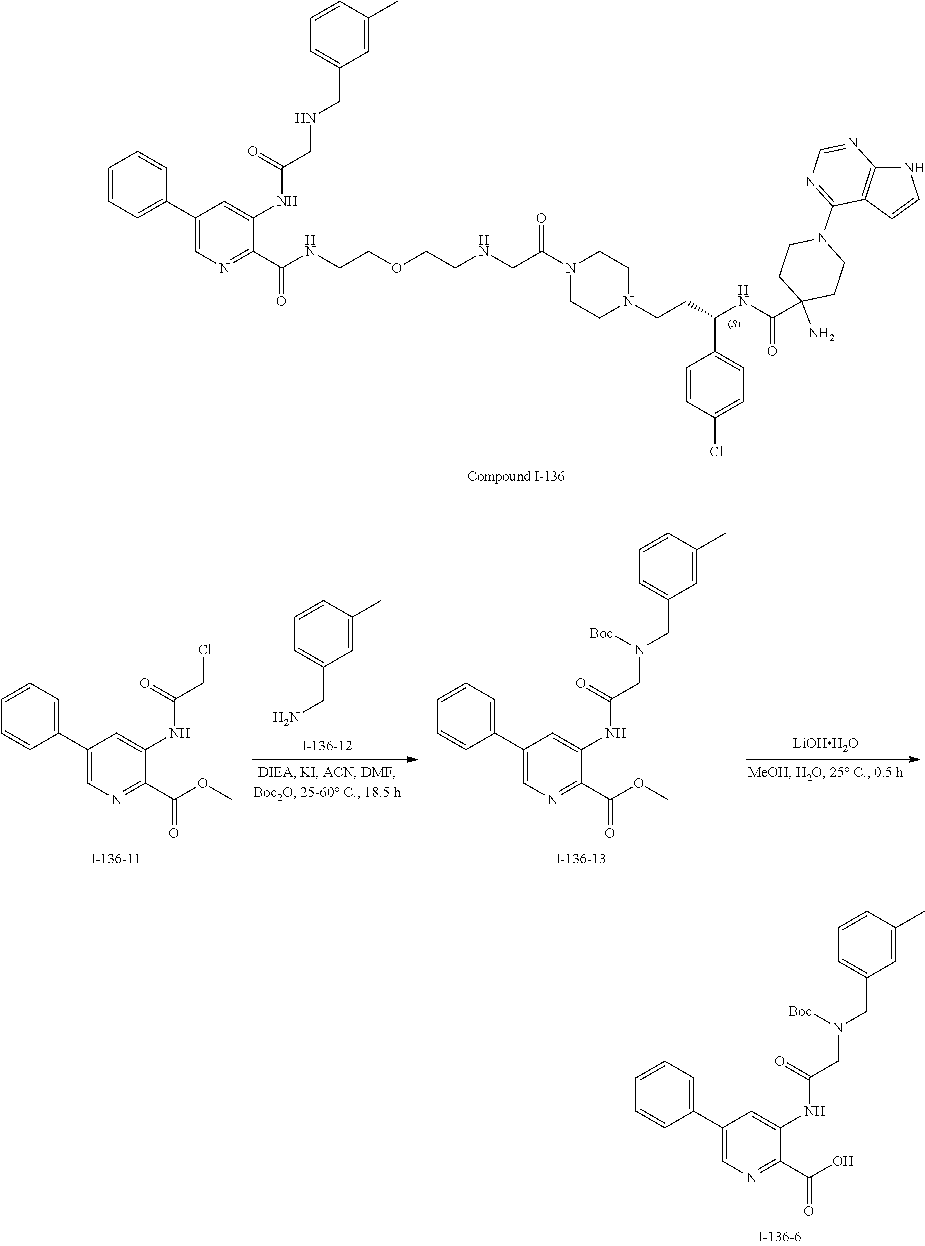

### Step 1: Synthesis of I-136-3

To a solution of I-136-1 (4 g. 16.79 mmol, 1 eq). HOAc (4.03 g. 67.15 mmol, 3.84 mL, 4 eq) in MeOH (40 mL) was added I-136-2 (6.85 g, 33.57 mmol, 2 eq). Then the mixture was stirred at 25° C. for 4 hrs. Then the mixture was added NaBH₃CN (6.33 g, 100.72 mmol, 6 eq) and stirred at 25° C. for 12 hrs. The reaction mixture was poured into water (100 mL) and extracted with DCM (50 mL*3). The organic layers were washed with brine (60 mL) and dried over Na₂SO₄, filtered and concentrated. I-136-3 (7.6 g, crude) was obtained as a colorless oil. LCMS (Method E): R^{t}=0.399 min, [M+H]⁺=325.2.

### Step 2: Synthesis of I-136-4

To a solution of I-136-3 (7.6 g, 23.43 mmol, 1 eq), TEA (4.74 g, 46.86 mmol, 6.52 mL, 2 eq), DMAP (286.24 mg, 2.34 mmol, 0.1 eq) in DCM (80 mL) was added Boc₂O (7.67 g, 35.14 mmol, 8.07 mL, 1.5 eq). Then the mixture was stirred at 25° C. for 1 h. The reaction mixture was poured into water (150 mL) and extracted with DCM (60 Ml*3). The organic layers were dried over Na₂SO₄, filtered and concentrated. The residue was purified by flash silica gel chromatography (ISCO®; 330 g SepaFlash® Silica Flash Column, Eluent of 0˜50% Ethyl acetate/Petroleum ether gradient @ 150 mL/min) to give I-136-4 (3.8 g, 8.95 mmol, 38.21% yield) as a colorless oil. LCMS (Method E): R^{t}=0.584 min, [M+H]⁺=325.3. ¹H NMR (400 MHZ, METHANOL-d4) δ=7.42-7.24 (m, 5H), 5.07 (s, 2H), 4.21-4.12 (m, 2H), 4.05-3.94 (m, 2H), 3.62-3.52 (m, 2H), 3.51-3.40 (m, 4H), 3.30-3.21 (m, 2H), 1.52-1.36 (m, 9H), 1.24 (dt, J=2.4, 7.2 Hz, 3H).

### Step 3: Synthesis of I-136-5

To a solution of I-136-4 (3.2 g, 7.54 mmol, 1 eq) in MeOH (32 mL) was added Pd(OH)₂/C (1.5 g, 20% purity). The mixture was purged with H₂ for three times and stirred at 25° C. for 2 h under H₂ atmosphere under 15 psi. The mixture was filtered and the filtrate was concentrated to give I-136-5 (2 g, 6.89 mmol, 91.37% yield) as a colorless oil. LCMS (Method E): R^{t}=0.374 min, [M+H]⁺=291.1. 1H NMR (400 MHZ, METHANOL-d4) δ=4.24-4.13 (m, 2H), 4.02 (d, J=6.4 Hz, 2H), 3.64-3.54 (m, 2H), 3.50-3.44 (m, 4H), 2.82-2.70 (m, 2H), 1.48-1.41 (m, 9H), 1.32-1.23 (m, 3H).

### Step 4: Synthesis of I-136-13

To a solution of I-136-12 (596.50 mg, 4.92 mmol, 617.49 μL, 1.5 eq) in ACN (10 mL) and DMF (10 mL) under N₂ atmosphere was added DIEA (1.70 g, 13.13 mmol, 2.29 mL, 4 eq), the reaction mixture was stirred at 25° C. for 0.5 h. Then KI (272.38 mg, 1.64 mmol, 0.5 eq) and I-136-11 (1 g, 3.28 mmol, 1 eq) was added and the mixture was stirred at 60° C. for 16 h. The reaction mixture was cooled to 25° C., Then Boc₂O (1.07 g, 4.92 mmol, 1.13 mL, 1.5 eq) was added to the reaction mixture and stirred for 2 h at 25° C.

The reaction mixture was concentrated. The crude product was purified by reverse-phase HPLC (0.1% NH₃·H₂O:ACN: 0-66%) to give I-136-13 (770 mg, 1.46 mmol, 44.57% yield, 93% purity) as a black oil. LCMS (Method E): R^{t}=0.644 min, [M+H]⁺=490.2. ¹H NMR (400 MHZ, METHANOL-d4) δ=9.26 (br s, 1H), 8.61 (s, 1H), 7.71 (br d, J=7.2 Hz, 2H), 7.58-7.44 (m, 3H), 7.20-7.07 (m, 3H), 7.02 (br d, J=7.6 Hz, 1H), 4.60 (s, 2H), 4.10-4.00 (m, 5H), 2.25 (s, 3H), 1.60-1.44 (m, 9H).

### Step 5: Synthesis of I-136-6

To a solution of I-136-13 (750 mg, 1.53 mmol, 1 eq) in MeOH (4 mL) was added LiOH·H₂O (321.44 mg, 7.66 mmol, 5 eq) in H₂O (1 mL). The mixture was stirred at 25° C. for 0.5 hr. The mixture was adjusted to pH=5 by HCl (1 M) and the solid was generated. The solid was filtered and concentrated to give I-136-6 (650 mg, 1.34 mmol, 87.44% yield, 98% purity) as a brown solid. R^{t}=0.425 min, [M+H]⁺=474.1. ¹H NMR (400 MHZ, DMSO-d₆) δ=14.30-14.02 (m, 1H), 9.21 (br d, J=5.6 Hz, 1H), 8.49 (br s, 1H), 7.69 (br d, J=7.6 Hz, 2H), 7.63-7.52 (m, 2H), 7.50-7.41 (m, 1H), 7.29-7.18 (m, 1H), 7.14-6.99 (m, 3H), 4.51 (s, 2H), 4.01-3.85 (m, 2H), 2.29 (s, 3H), 1.42-1.31 (m, 9H).

### Step 6: Synthesis of I-136-7

To a solution of I-136-6 (200 mg, 420.58 μmol, 1 eq) and I-136-5 (366.35 mg, 1.26 mmol, 3 eq) in DMF (2 mL) was added EDCI (241.88 mg, 1.26 mmol, 3 eq), HOAt (171.74 mg, 1.26 mmol, 3 eq) and NMM (212.70 mg, 2.10 mmol, 231.20 μL, 5 eq). The mixture was stirred at 25° C. for 17 h. The mixture was washed with water (10 mL) and extracted with EtOAc (3 mL*4). The combined organic layers were dried over Na₂SO₄, filtered and concentrated to give I-136-7 (500 mg, crude) as a brown oil. LCMS (Method E): R^{t}=0.725 min, [M+H]⁺=748.3.

### Step 7: Synthesis of I-136-8

To a solution of I-136-7 (150 mg, 200.57 μmol, 1 eq) in MeOH (2 mL) was added LiOH H₂O (42.08 mg, 1.00 mmol, 5 eq) in H₂O (0.5 mL). The mixture was stirred at 25° C. for 1 h. The mixture was adjusted to pH=6 by HCl (1 M) and extracted with EtOAc (2 mL*3). The combined organic layers were dried over Na₂SO₄, filtered and concentrated to give I-136-8 (80 mg, 75.57 μmol, 37.68% yield, 68% purity) as a brown oil. LCMS (Method E): R^{t}=0.668 min, [M+H]⁺=720.4.

### Step 8: Synthesis of I-136-10

To a solution of I-136-8 (80 mg, 111.14 μmol, 1 eq) in DMF (0.5 mL) was added EDCI (63.92 mg, 333.42 μmol, 3 eq), HOAt (45.38 mg, 333.42 μmol, 3 eq) and NMM (56.21 mg, 555.69 μmol, 61.09 μL, 5 eq). The mixture was stirred at 25° C. for 10 min and I-136-9 (86.28 mg, 144.48 μmol, 1.3 eq) was added. The resulting mixture was stirred at 25° C. for 20 min. The mixture was added drop wise to water (3 mL) and solid was generated. The mixture was filtered and the solid was concentrated. The crude product was purified by reverse-phase HPLC (0.1% FA condition) and the filtrate was concentrated to give I-136-10 (65 mg, 49.54 μmol, 44.57% yield, 99% purity) as a yellow oil. LCMS (Method B): R^{t}=0.815 min, [M+H]⁺=1298.9.

### Step 9: Synthesis of I-136

To a solution of I-136-10 (45 mg, 34.64 μmol, 1 eq) in dioxane (0.5 mL) was added HCl/dioxane (0.5 mL) (4 M). The mixture was stirred at 25° C. for 0.5 hr. The reaction mixture was concentrated to give I-136 (18.5 mg, 16.98 μmol, 49.01% yield, 95% purity, HCl salt) as yellow solid. LCMS (Method C): R^{t}=1.016 min, [M+H]⁺=998.6. ¹H NMR (400 MHZ, METHANOL-d4) δ=9.25 (d, J=2.0 Hz, 1H), 8.67 (d, J=2.0 Hz, 1H), 8.39 (s, 1H), 7.72 (d, J=7.2 Hz, 2H), 7.59-7.46 (m, 3H), 7.46-7.33 (m, 8H), 7.29 (br d, J=6.0 Hz, 1H), 7.01 (br d, J=3.2 Hz, 1H), 5.10-5.00 (m, 1H), 4.68-4.48 (m, 3H), 4.31 (s, 3H), 4.24-4.12 (m, 3H), 4.09-3.81 (m, 6H), 3.79-3.48 (m, 9H), 3.28-3.07 (m, 4H), 2.95-2.65 (m, 2H), 2.61-2.46 (m, 1H), 2.39 (s, 3H), 2.36-2.20 (m, 2H), 2.13-2.00 (m, 1H). SFC: Method details: “Column: Chiralcel OD-3 50*4.6 mm I.D., 3 um; Mobile phase: Phase A for CO₂, and Phase B for EtOH (0.05% DEA); Gradient elution: 50% EtOH (0.05% DEA) in CO₂: Flow rate: 3 mL/min; Detector: PDA; Column Temp: 35° C.: R^{t}=1.475 min; f02.

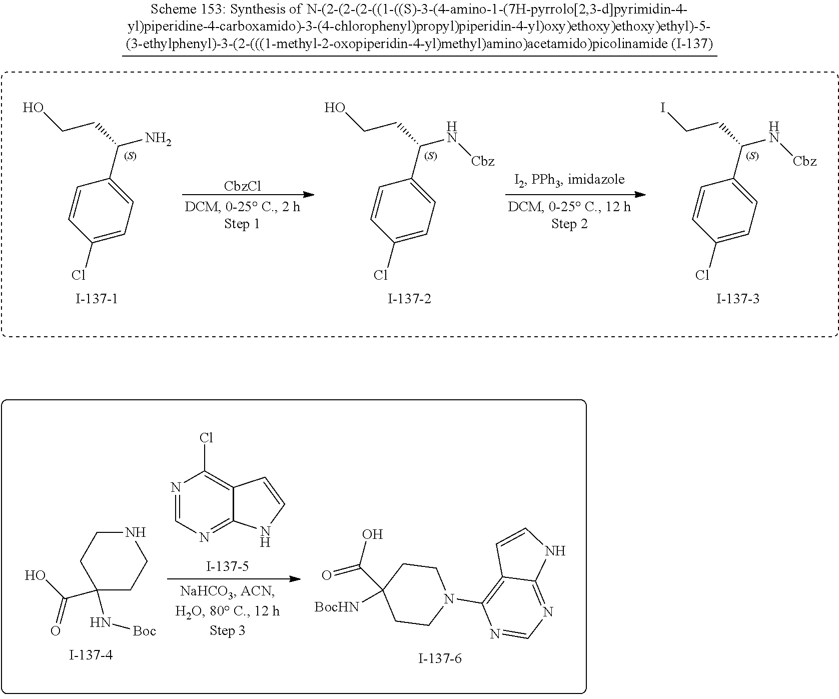

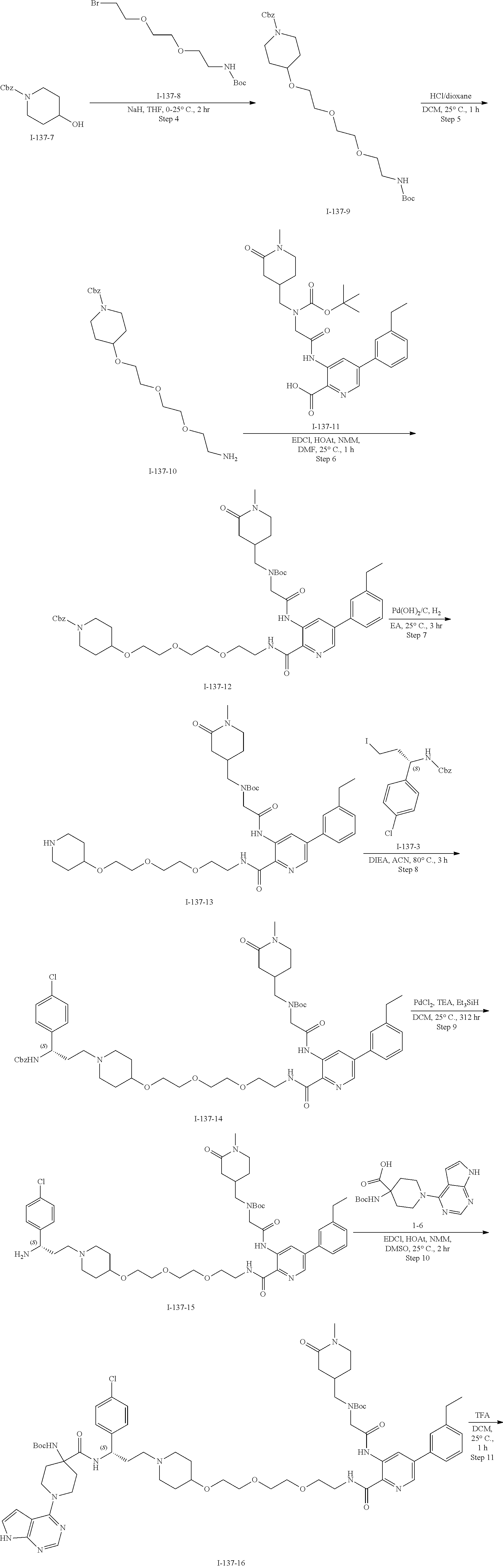

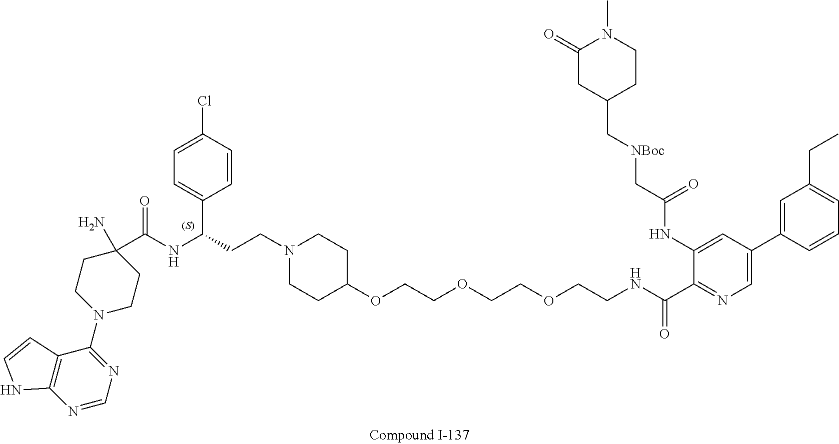

### Step 1: Synthesis of I-137-2

To a solution of I-137-1 (0.5 g, 2.69 mmol, 1 eq) in DCM (5 mL) was added DIEA (1.04 g, 8.08 mmol, 1.41 mL, 3 eq). After addition, the mixture was stirred at 25° C. for 0.25 hr, and then benzyl carbonochloridate (551.34 mg, 3.23 mmol, 461.37 μL, 1.2 eq) was added dropwise at 0° C. The mixture was stirred at 25° C. for 1.75 hr. The reaction mixture was quenched by addition water 10 mL at 25° C., and then extracted with DCM 15 mL (5 mL*3). Then dried over Na₂SO₄, filtered and concentrated under reduced pressure to give a residue. The residue was purified by flash silica gel chromatography (ISCO®; 40 g SepaFlash® Silica Flash Column, Eluent of 0˜60% Ethyl acetate/Petroleum ether gradient @ 60 mL/min) to afford I-137-2 (0.5 g, 1.34 mmol, 49.93% yield, 86% purity) was obtained as a white solid. LCMS (Method G): R^{t}=0.601 min, [M+Na]⁺=342.2.

### Step 2: Synthesis of I-137-3

To a solution of PPh₃ (328.08 mg, 1.25 mmol, 2 eq), IMIDAZOLE (170.31 mg, 2.50 mmol, 4 eq) in DCM (2 mL) at 25° cover 10 min. And then I2 (317.48 mg, 1.25 mmol, 251.97 μL, 2 eq) was added dropwise at 0° C. The resulting mixture was stirred at 0° C. for 20 min, and then I-137-2 (0.2 g, 625.43 μmol, 1 eq) was added dropwise at 25° C. The resulting mixture was stirred at 25° C. for 11.5 hrs. The reaction mixture was poured into water (10 mL) and saturated Na₂SO₃ aqueous (10 mL), the organic layer was washed with brine (5 mL), dried over anhydrous sodium sulfate, filtered and concentrated to give residue. The residue was purified by flash silica gel chromatography (ISCO®; 40 g SepaFlash® Silica Flash Column, Eluent of 0˜60% Ethyl acetate/Petroleum ether gradient (@ 60 mL/min) to afford I-137-3 (200 mg, 465.46 μmol, 74.42% yield, 100% purity) as a white solid. LCMS (Method E): R^{t}=0.625 min, [M+H]⁺=430.0.

### Step 3: Synthesis of I-137-6

To a solution of I-137-4 (0.1 g, 409.36 μmol, 1 eq) in H₂O (0.5 mL) and ACN (1 mL) was added NaHCO₃ (68.78 mg, 818.71 μmol, 2 eq) and I-137-5 (75.44 mg, 491.23 μmol, 1.2 eq). The mixture was stirred at 80° C. for 12 hrs. The reaction mixture was extracted by EA (10 mL×4). The aqueous layer was concentrated under oil pump and methanol (10 mL) was added. The mixture was stirred at 25° C. for 30 min and filtered. The filtrate was concentrated and dissolved into water (10 mL), 4M HCl (˜10 mL) was added into stirred solution to adjusted pH=4, white solid was formed. The suspension was filtered to afford white solid. The filter cake was washed by water (10 mL) and concentrated to afford I-137-6 (0.12 g, 332.05 μmol, 81.11% yield, 100% purity) as white solid. LCMS (Method E): R^{t}=0.395 min, [M+H]⁺=362.3.

### Step 4: Synthesis of I-137-9

To a solution of I-137-7 (0.5 g, 2.13 mmol, 1 eq) in THF (5 mL) was added NaH (254.99 mg, 6.38 mmol, 60% purity, 3 eq) at 0° C. After addition, the mixture was stirred at this temperature for 0.5 hr at 0° C., and then I-137-8 (1.33 g, 4.25 mmol, 2 eq). The resulting mixture was stirred at 25° C. for 1.5 hr. The mixture was quenched by NH₄Cl (20 mL) and extracted with EA (5 mL*3). The combined organic layers were dried over anhydrous Na₂SO₄, filtered and concentrated to give a residue. The residue was purified by flash silica gel chromatography (ISCO®; 40 g SepaFlash® Silica Flash Column, Eluent of 0˜100% Ethyl acetate/Petroleum ether gradient (40 mL/min) to afford I-137-9 (420 mg, 693.15 μmol, 32.62% yield, 77% purity) as colorless oil. LCMS (Method E): R^{t}=0.573 min, [M+H]⁺=467.4. ¹H NMR (400 MHZ, CHLOROFORM-d) δ=7.39-7.28 (m, 4H), 5.12 (s, 2H), 5.06 (s, 1H), 3.95-3.71 (m, 3H), 3.69-3.58 (m, 8H), 3.57-3.50 (m, 3H), 3.32-3.30 (d, J=4.8 Hz, 2H), 3.23-3.13 (m, 2H), 1.85 (s, 4H), 1.44 (s, 9H).

### Step 5: Synthesis of I-137-10

To a solution of I-137-9 (400 mg, 857.33 μmol, 1 eq) in DCM (4 mL) was added HCl/dioxane (4 M, 4.00 mL, 18.66 eq). The mixture was stirred at 25° C. for 1 hr. The reaction mixture was concentrated under reduced pressure to give a residue. The residue was purified by prep-HPLC (column: Phenomenex Luna C18 150*25 mm*10 um; mobile phase: [water (TFA)-ACN]; gradient: 15%-45% B over 9 min), the target peak was concentrated and lyophilized to afford I-137-10 (320 mg, 746.57 μmol, 87.08% yield, 94% purity, HCl) as colorless oil. LCMS (Method E): R^{t}=0.424 min, [M+H]⁺=367.1. ¹H NMR (400 MHZ, METHANOL-d4) δ=7.43-7.25 (m, 5H), 5.11 (s, 2H), 3.81 (m, 2H), 3.68 (s, 3H), 3.67-3.65 (m, 8H), 3.18-3.07 (m, 4H), 1.93-1.81 (m, 2H), 1.56-1.40 (m, 2H).

### Step 6: Synthesis of I-137-12

To a solution of I-137-11 (150 mg, 285.93 μmol, 1 eq) in DMF (2 mL) was added HOAt (38.92 mg, 285.93 μmol, 40.00 μL, 1 eq), EDCI (164.44 mg, 857.78 μmol, 3 eq), NMM (202.45 mg, 2.00 mmol, 220.05 μL, 7 eq), and I-137-10 (115.20 mg, 285.93 μmol, 1 eq, HCl). The mixture was stirred at 25° C. for 1 hr. The reaction mixture was quenched by addition water 5 mL at 25° C., and then extracted with EA 15 mL (5 mL*3). Then dried over Na₂SO₄, filtered and concentrated under reduced pressure to give a residue. The residue was purified by reversed phase (FA condition), then concentrated to remove organic solvents and lyophilized to give I-137-12 (200 mg, 229.08 μmol, 80.12% yield, 100% purity) as colorless oil. LCMS (Method E): R^{t}=0.659 min, [M+H]⁺=873.6.

### Step 7: Synthesis of I-137-13

To a solution of Pd(OH)₂/C (100 mg, 50% purity) in EtOAc (2 mL) was added I-137-12 (200 mg, 229.08 μmol, 1 eq). The mixture was stirred at 25° C. for 3 hrs under H₂ (15 psi) atmosphere. The reaction mixture was filtered to remove Pd(OH)₂/C, and was filtered and concentrated to afford I-137-13 (150 mg, crude) as colorless oil. LCMS (Method E): R^{t}=0.516 min, [2M+H]⁺=739.4.

### Step 8: Synthesis of I-137-14

To a solution of I-137-13 (199.48 mg, 269.97 μmol, 1 eq) in ACN (2 mL) was added DIEA (69.78 mg, 539.94 μmol, 94.05 μL, 2 eq) and I-137-3 (116 mg, 269.97 μmol, 1 eq). The mixture was stirred at 80° C. for 3 hrs. The reaction mixture was quenched by addition water 5 mL at 25° C., and then extracted with EA 15 mL (5 mL*3). Then dried over Na₂SO₄, filtered and concentrated under reduced pressure to give a residue. The residue was purified by reversed phase (FA condition), then concentrated to remove organic solvents and lyophilized to give I-137-14 (180 mg, 168.52 μmol, 62.42% yield, 97.43% purity) as colorless oil. LCMS (Method E): R^{t}=0.587 min, [M+H]⁺=1040.5. SFC: R^{t}=1.151 min, 1.435 min, ee %=0.004%. ¹H NMR (400 MHZ, DMSO-d₆) δ=12.58-12.32 (m, 1H), 9.28-9.27 (d, J=4.8 Hz, 1H), 9.07 (m, 1H), 8.63 (s, 1H), 7.93-7.91 (d, J=8.4 Hz, 1H), 7.56-7.51 (m, 2H), 7.46 (m, 1H), 7.38-7.28 (m, 10H), 5.76 (s, 2H), 4.98 (m, 2H), 4.61-4.49 (m, 1H), 4.03 (s, 3H), 3.58-3.53 (m, 8H), 3.51-3.45 (m, 10H), 2.79 (s, 3H), 2.73-2.67 (m, 2H), 2.63-2.62 (d, J=5.6 Hz, 2H), 2.23 (m, 3H), 2.02 (s, 2H), 1.94-1.82 (m, 2H), 1.81-1.71 (m, 4H), 1.52-1.32 (m, 9H), 1.25-1.21 (m, 3H).

### Step 9: Synthesis of I-137-15

To a solution of I-137-14 (40 mg, 38.44 μmol, 1 eq) in DCM (0.5 mL) was added PdCl2 (2.04 mg, 11.53 μmol, 0.3 eq) and TEA (11.67 mg, 115.31 μmol, 16.05 μL, 3 eq). The reaction mixture was degassed with N₂. Then a solution of Et3SiH (44.69 mg, 384.36 μmol, 61.39 μL, 10 eq) in DCM (0.2 mL) was added dropwise at 10° C. The reaction mixture was stirred at 25° C. for 12 hrs. The reaction mixture was quenched by addition NaHCO₃ (5 mL) at 25° C., and then extracted with EA 15 mL (5 mL*3). Then dried over Na₂SO₄, filtered and concentrated under reduced pressure to give a residue. The residue was purified by flash silica gel chromatography (ISCO®; 12 g SepaFlash® Silica Flash Column, Eluent of 0˜20% Methanol/Ethyl acetate (@ 20 mL/min) to afford I-137-15 (25 mg, 26.13 μmol, 67.97% yield, 94.74% purity) as colorless oil. LCMS (Method E): R^{t}=0.513 min, [(M-55)/2+H]⁺=426.3.

### Step 10: Synthesis of I-137-16

To a solution of I-137-6 (11.96 mg, 33.09 μmol, 1 eq) in DMSO (0.5 mL) was added HOAt (6.76 mg, 49.64 μmol, 6.94 μL, 1.5 eq), EDCI (9.52 mg, 49.64 μmol, 1.5 eq), NMM (16.74 mg, 165.46 μmol, 18.19 μL, 5 eq), and I-137-15 (30 mg, 33.09 μmol, 1 eq). The mixture was stirred at 25° C. for 2 hrs. The reaction mixture was quenched by addition water 5 mL at 25° C., and then extracted with EA 15 mL (5 mL*3). Then dried over Na₂SO₄, filtered and concentrated under reduced pressure to give a residue. The residue was purified by prep-HPLC (column: C18 150×30 mm; mobile phase: [water (FA)-ACN]; gradient: 32%-62% B over 7 min), the target peak was concentrated and lyophilized to afford I-137-16 (30 mg, 21.19 μmol, 64.04% yield, 88.3% purity) as yellow solid. LCMS (Method E): R^{t}=0.534 min, [M+H]⁺=1249.8.

### Step 11: Synthesis of I-137

To a solution of I-137-16 (20 mg, 16.00 μmol, 1 eq) in DCM (0.1 mL) was added TFA (153.50 mg, 1.35 mmol, 0.1 mL, 84.13 eq). The mixture was stirred at 25° C. for 1 hr. The reaction mixture was concentrated under reduced pressure to give a residue. The residue was purified by prep-HPLC (column:

Phenomenex Luna C18 150*25 mm*10 um; mobile phase: [water (TFA)-ACN]; gradient: 15%-45% B over 9 min), the target peak was concentrated and lyophilized to afford I-137 (15.74 mg, 13.53 μmol, 84.53% yield, 100% purity, TFA) as an off-white solid. LCMS (Method E): R^{t}=0.405 min, [M+H]⁺=1049.9. SFC: R^{t}=3.310 min, 3.582 min, ee %=4.766%. ¹H NMR (400 MHZ, CHLOROFORM-d) δ=13.64-13.48 (m, 1H), 12.58 (d, J=5.6 Hz, 1H), 9.17-8.99 (m, 2H), 8.58 (s, 1H), 8.50 (d, J=1.6 Hz, 1H), 8.19 (s, 1H), 7.42-7.40 (d, J=6.8 Hz, 2H), 7.39-7.35 (m, 1H), 7.27-7.22 (m, 5H), 7.13 (d, J=3.2 Hz, 1H), 6.55 (d, J=3.6 Hz, 1H), 5.05-4.95 (m, 1H), 4.41-4.29 (m, 2H), 4.20-4.14 (m, 1H), 4.06 (s, 2H), 3.99-3.77 (m, 2H), 3.73-3.53 (m, 12H), 3.49 (s, 2H), 3.32-3.26 (m, 2H), 3.20-3.03 (m, 7H), 2.96-2.85 (m, 3H), 2.83-2.80 (d, J=8.8 Hz, 4H), 2.72 (m, 4H), 2.62-2.56 (m, 2H), 2.51-2.45 (m, 2H), 2.27-2.19 (m, 3H), 2.05-1.99 (m, 2H), 1.66 (s, 2H), 1.30-1.26 (m, 3H).

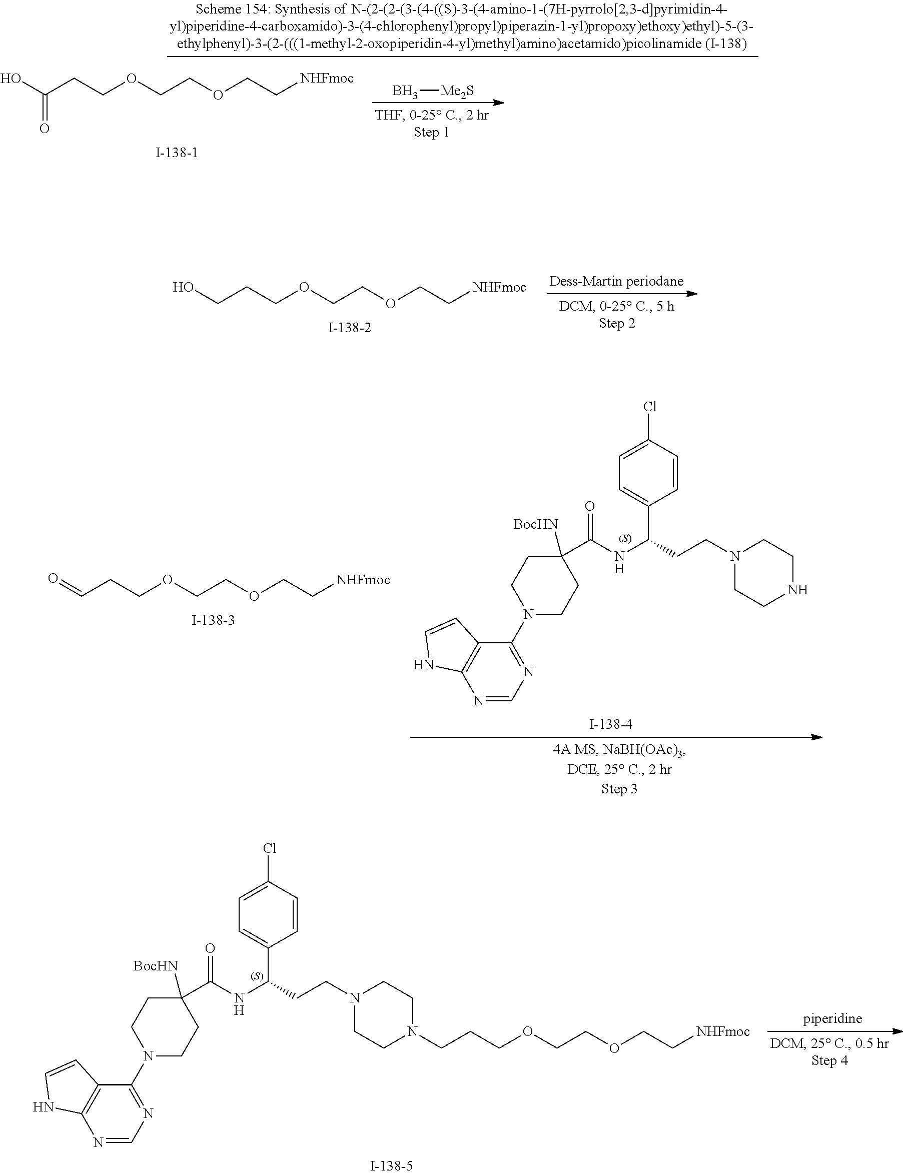

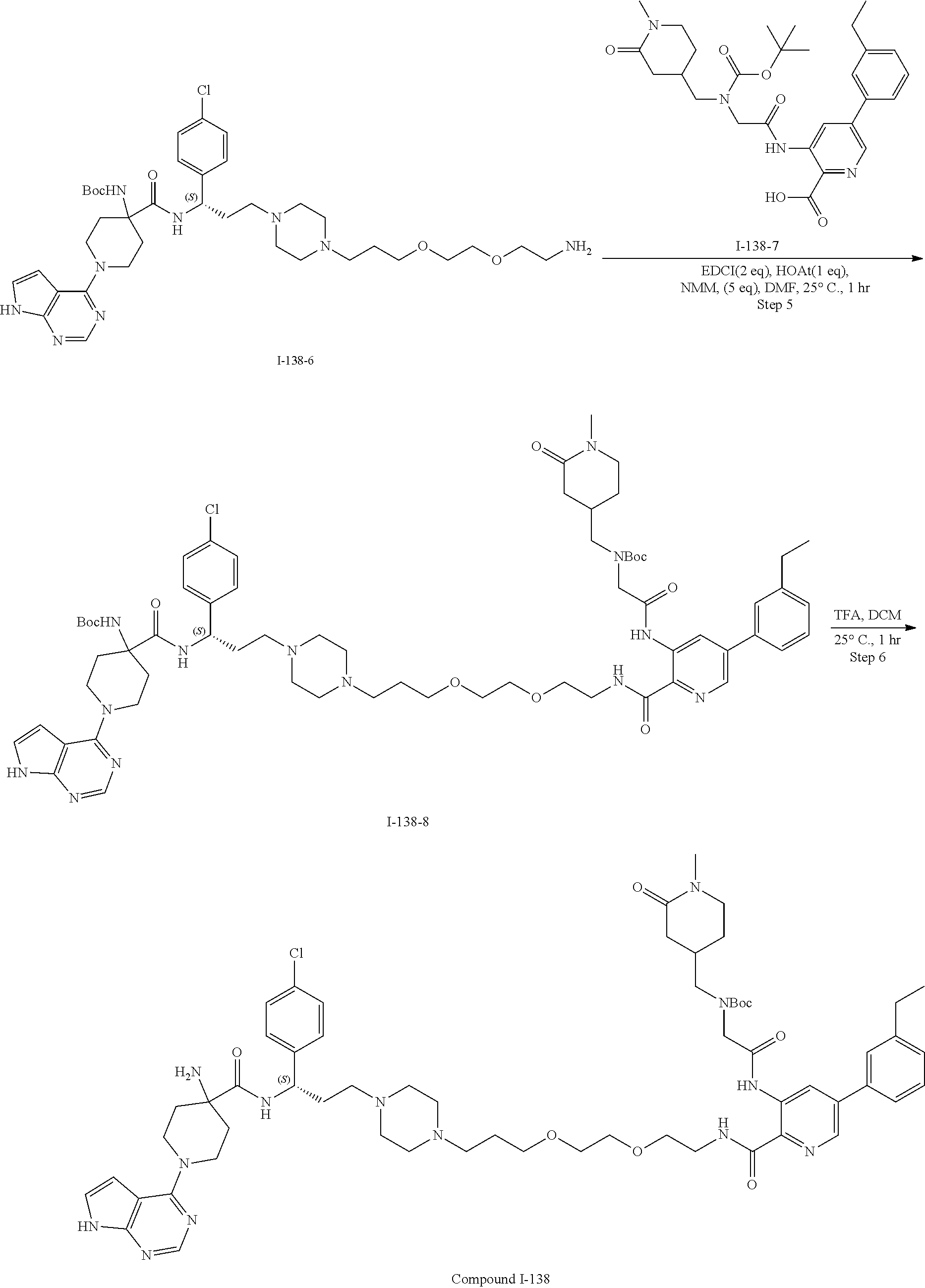

### Step 1: Synthesis of I-138-2

To a solution of I-138-1 (1 g, 2.50 mmol, 1 eq) in THF (10 mL) was added BH₃-Me₂S (10M, 751.06 ML, 3 eq) at 0° C., and purged with N₂ for 3 times. The mixture was stirred at 25° C. for 2 hrs. The mixture was quenched by HCl (20 mL, 1M) and extracted with EA (10 mL*3). The combined organic layers were dried over anhydrous Na₂SO₄, filtered and concentrated to give I-138-2 (900 mg, crude) as white solid. LCMS (Method E): R^{t}=0.525 min, [M+H]⁺=386.2.

### Step 2: Synthesis of I-138-3

To a solution of 9 I-138-2 (0.2 g, 518.87 μmol, 1 eq) in DCM (2 mL) was added (1,1-diacetoxy-3-oxo-1,2-benziodoxol-1-yl) acetate (330.11 mg, 778.31 μmol, 241.13 μL, 1.5 eq) at 0° C. The mixture was stirred at 25° C. for 5 hrs. The mixture was poured into saturated aqueous NaHCO₃ solution (10 mL) and was extracted with dichloromethane (5 mL*2). The combined organic layers were dried over anhydrous Na₂SO₄, filtered and concentrated. The residue was purified by flash silica gel chromatography (ISCO®; 20 g SepaFlash® Silica Flash Column, Eluent of 0˜100% Ethyl acetate/Petroleum ether gradient @ 40 mL/min) to afford I-138-3 (150 mg, 356.89 μmol, 68.78% yield, 91.23% purity) as a colorless solid. LCMS (Method E): R^{t}=0.542 min, [M+H]⁺=384.3. ¹H NMR (400 MHz, DMSO-d₆) δ=9.65-9.61 (m, 1H), 7.90-7.88 (d, J=7.2 Hz, 2H), 7.70-7.68 (d, J=7.2 Hz, 2H), 7.45-7.38 (m, 2H), 7.36-7.24 (m, 3H), 4.30-4.28 (d, J=6.8 Hz, 2H), 4.25-4.15 (m, 1H), 3.71 (m, 2H), 3.49 (s, 4H), 3.41-3.37 (m, 2H), 3.12 (m, 2H), 2.59 (m, 2H).

### Step 3: Synthesis of I-138-5

To a solution of I-138-4 (141.58 mg, 237.09 μmol, 1 eq) in DCE (2 mL) was added 4 A MS (20 mg). The mixture was stirred at 25° C. for 0.25 hr and then I-138-3 (100 mg, 260.80 μmol, 1.1 eq) was added dropwise at 25° C., the mixture was stirred at 25° C. for 0.75 hr. And sodium: triacetoxyboranuide (125.62 mg, 592.73 μmol, 2.5 eq) was added. The mixture was stirred at 25° C. for 1 hr. The reaction mixture was quenched by addition water 5 mL at 25° C., and then extracted with DCM 15 mL (5 mL*3). Then dried over Na₂SO₄, filtered and concentrated under reduced pressure to give a residue. The residue was purified by reversed phase (FA condition), then concentrated to remove organic solvents and lyophilized to give I-138-5 (170 mg, 176.24 μmol, 74.34% yield, 100% purity) as a colorless solid. LCMS (Method E): R^{t}=0.518 min, [M+H]⁺=964.5. ¹H NMR (400 MHZ, DMSO-d₆) δ=11.68 (s, 1H), 8.22-8.20 (d, J=7.6 Hz, 1H), 8.12 (s, 1H), 7.87 (d, J=7.6 Hz, 2H), 7.69-7.67 (d, J=7.6 Hz, 2H), 7.43-7.38 (m, 2H), 7.35-7.28 (m, 8H), 7.15 (d, J=2.0 Hz, 1H), 6.59 (d, J=3.2 Hz, 1H), 4.82 (m, 1H), 4.28 (d, J=6.8 Hz, 2H), 4.24-4.14 (m, 4H), 3.67-3.53 (m, 4H), 3.47 (d, J=7.2 Hz, 4H), 3.42-3.38 (m, 3H), 3.12 (d, J=5.6 Hz, 2H), 2.34-2.23 (m, 8H), 2.19 (d, J=6.4 Hz, 2H), 1.98 (d, J=4.8 Hz, 4H), 1.82-1.75 (m, 2H), 1.63-1.56 (m, 2H), 1.39 (s, 9H).

### Step 4: Synthesis of I-138-6

To a solution of I-138-5 (140 mg, 145.14 μmol, 1 eq) in DCM (2 mL) was added piperidine (1.72 g, 20.25 mmol, 2 mL, 139.53 eq). The mixture was stirred at 25° C. for 0.5 hr. The reaction mixture was concentrated under reduced pressure to give a residue. The residue was purified by flash silica gel chromatography (ISCO®; 20 g SepaFlash® Silica Flash Column, Eluent of 0˜50% Methanol/Ethyl acetate (@ 40 mL/min) to afford I-138-6 (100 mg, 132.35 μmol, 91.19% yield, 98.25% purity) as colorless oil. LCMS (Method E): R^{t}=0.397 min, [M+H]⁺=742.5. SFC: R^{t}=1.170 min, ee %=100%.

### Step 5: Synthesis of I-138-8

To a solution of I-138-7 (35.33 mg, 67.35 μmol, 1 eq) in DMF (0.5 mL) was added HOAt (9.17 mg, 67.35 μmol, 9.42 μL, 1 eq), EDCI (25.82 mg, 134.71 μmol, 2 eq), NMM (34.06 mg, 336.77 μmol, 37.03 μL, 5 eq), and I-138-6 (50 mg, 67.35 μmol, 1 eq). The mixture was stirred at 25° C. for 1 hr. The reaction mixture was quenched by addition water 5 mL at 25° C., and then extracted with EA 15 mL (5 mL*3). Then dried over Na₂SO₄, filtered and concentrated under reduced pressure to give I-138-8 (50 mg, crude) as yellow solid. LCMS (Method E): R^{t}=0.521 min, [M+H]⁺=1250.2.

### Step 6: Synthesis of I-138

To a solution of I-138-8 (25 mg, 20.02 μmol, 1 eq) in DCM (0.5 mL) was added TFA (127.92 mg, 1.12 mmol, 83.33 μL, 56.04 eq). The mixture was stirred at 25° C. for 1 hr. The reaction mixture was concentrated under reduced pressure to give a residue. The residue was purified by prep-HPLC (column: Phenomenex Luna C18 150*25 mm*10 um; mobile phase: [water (TFA)-ACN]; gradient: 15%-45% B over 9 min), the target peak was concentrated and lyophilized to afford I-138 (3.74 mg, 3.15 μmol, 15.73% yield, 97.88% purity, TFA) as off-white solid. LCMS (Method E): R^{t}=0.436 min, [M+H]⁺=1049.6. SFC: R^{t}=11.267 min, 13.860 min. ¹H NMR (400 MHZ, METHANOL-d4) δ=9.23 (d, J=2.0 Hz, 1H), 8.63 (d, J=2.0 Hz, 1H), 8.34 (s, 1H), 7.53-7.46 (m, 2H), 7.43 (m, 1H), 7.36-7.29 (m, 6H), 6.86 (d, J=3.6 Hz, 1H), 5.00 (m, 1H), 4.71-4.52 (m, 4H), 4.22 (s, 2H), 3.85-3.73 (m, 2H), 3.71-3.57 (m, 10H), 3.46-3.40 (m, 2H), 3.19-3.08 (m, 5H), 2.94 (s, 3H), 2.73 (m, 3H), 2.66-2.45 (m, 6H), 2.41-2.31 (m, 1H), 2.27-1.82 (m, 10H), 1.75-1.55 (m, 2H), 1.30-1.26 (m, 3H).

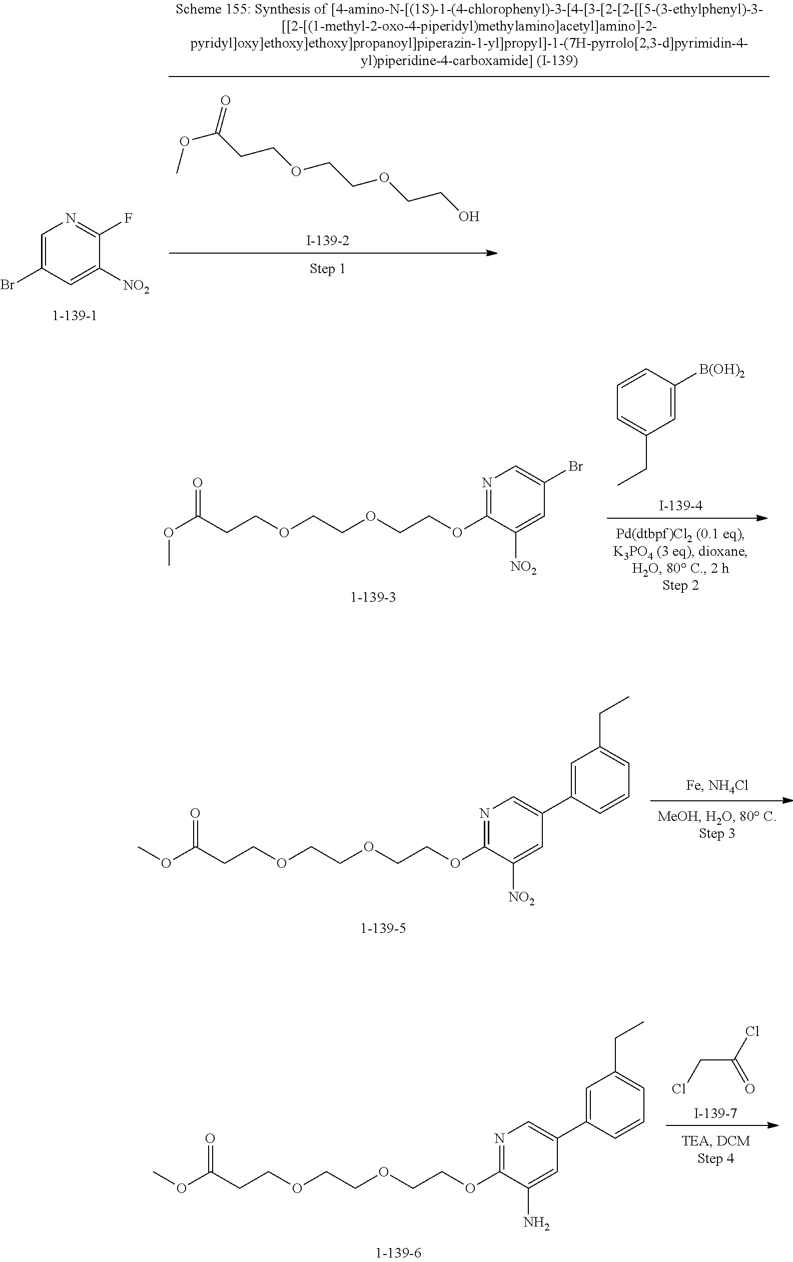

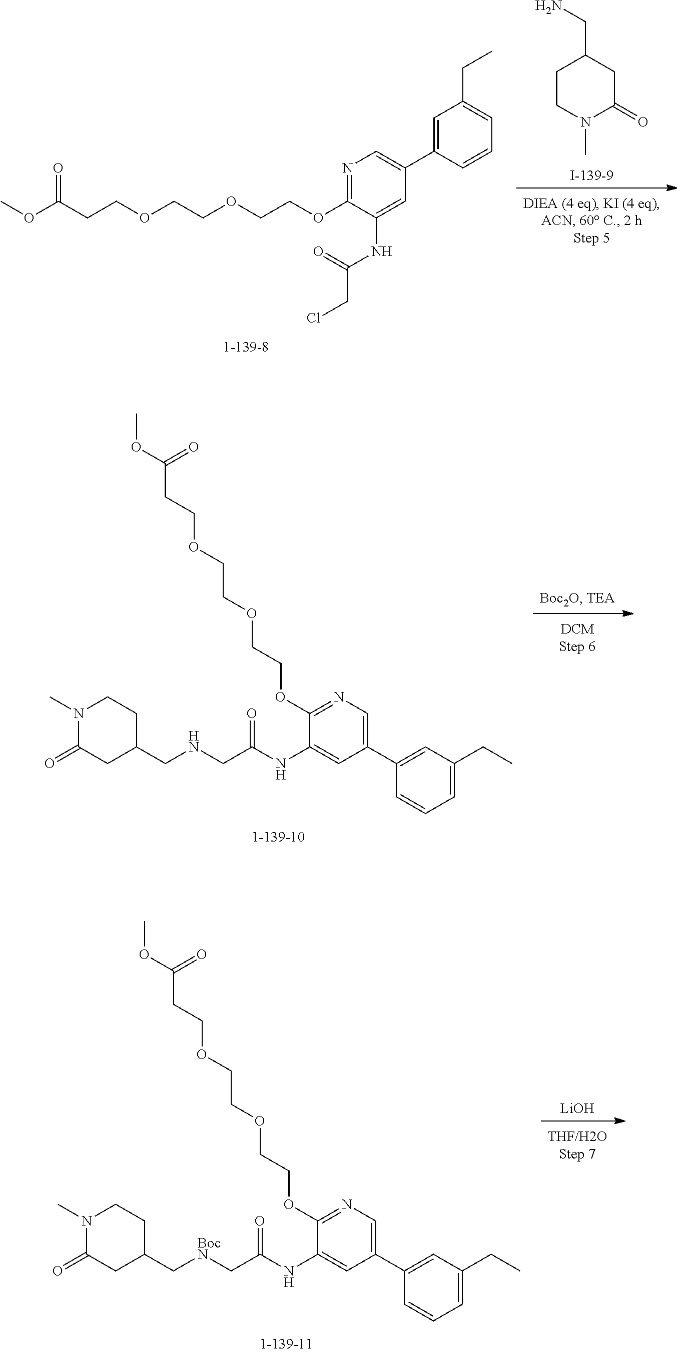

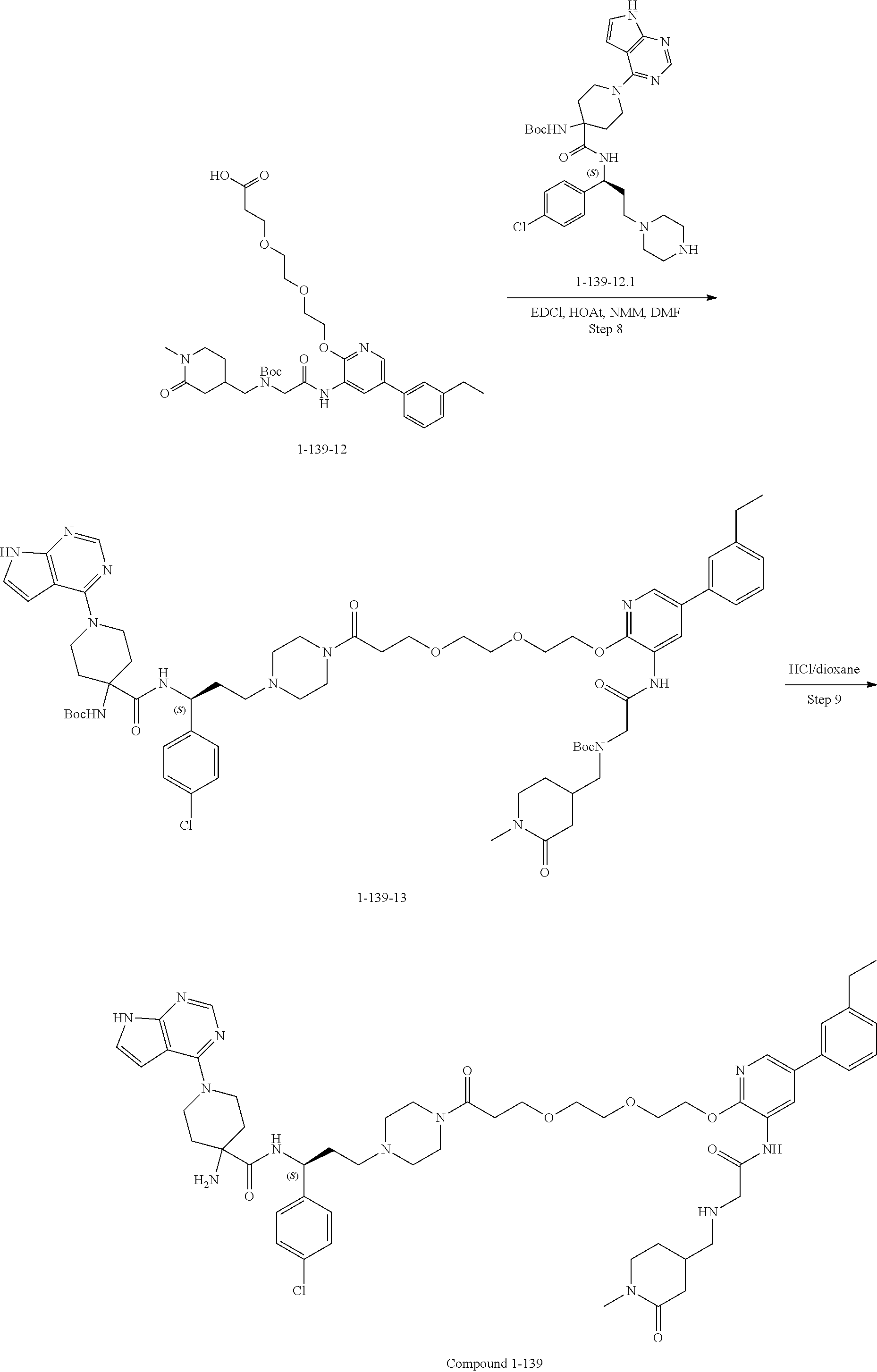

### Step 1: Synthesis of I-139-3

To a solution of I-139-2 (521.87 mg, 2.72 mmol, 1.2 eq) in MeCN (5 mL) was added DIEA (438.64 mg, 3.39 mmol, 591.16 μL, 1.5 eq), I-139-1 (500 mg, 2.26 mmol, 1 eq). The reaction mixture was stirred for 16 hr at 60° C. The reaction mixture was quenched with water (5 mL), extracted with EA (3 mL*3), the combined organic phase was dried by anhydrous Na₂SO₄, filtered and the filtrates was concentrated to give crude product. The crude product was purified by flash silica gel chromatography (ISCO®; 40 g SepaFlash® Silica Flash Column, Eluent of 0˜100% EA/PE @ 60 mL/min). After purification, then concentrated to give I-139-3 (380 mg, 966.46 μmol, 42.71% yield, N/A purity) as brown oil. LCMS (Method G): R^{t}=0.611 min, [M+H]⁺=393.1. ¹H NMR (400 MHZ, DMSO-d₆) δ=8.70 (d, J=2.4 Hz, 1H), 8.65 (d, J=2.4 Hz, 1H), 4.63-4.43 (m, 2H), 3.79-3.72 (m, 2H), 3.60 (t, J=6.4 Hz, 2H), 3.58 (s, 3H), 3.57-3.47 (m, 4H), 2.50 (br s, 2H).

### Step 2: Synthesis of I-139-5

To a solution of I-139-3 (380 mg, 966.46 μmol, 1 eq) and I-139-4 (173.94 mg, 1.16 mmol, 1.2 eq), K3PO4 (615.44 mg, 2.90 mmol, 3 eq) in dioxane (3.75 mL), H₂O (0.75 mL) was added Pd(dtbpf)Cl₂ (62.99 mg, 96.65 μmol, 0.1 eq) under N₂, the reaction mixture was degassed and purged with N₂ for 3 times, and then the mixture was stirred at 80° C. for 2 hrs. The reaction mixture was quenched with water (10 mL), extracted with EA (5 mL*3), the combined organic phase was dried by anhydrous Na₂SO₄, filtered and the filtrates was concentrated to give crude product. The crude product was purified by flash silica gel chromatography (ISCO®; 10 g SepaFlash® Silica Flash Column, Eluent of 0˜100% EA/PE @ 60 mL/min). After purification, then concentrated to give product I-139-5 (375 mg, 896.19 μmol, 92.73% yield, N/A purity) as brown oil. LCMS (Method E): R^{t}=0.630 min, [M+H]⁺=419.2. ¹H NMR (400 MHZ, CHLOROFORM-d) δ=8.59 (d, J=2.4 Hz, 1H), 8.47 (d, J=2.4 Hz, 1H), 7.45-7.35 (m, 3H), 7.30-7.27 (m, 1H), 4.70-4.68 (m, 2H), 3.94-3.85 (m, 2H), 3.81-3.74 (m, 4H), 3.71-3.63 (m, 5H), 2.74 (q, J=7.4 Hz, 2H), 2.62 (t. J=6.5 Hz, 2H), 1.30 (t. J=7.4 Hz, 3H).

### Step 3: Synthesis of I-139-6

To a solution of I-139-5 (420 mg, 1.00 mmol, 1 eq) in MeOH (3 mL) and H₂O (1 mL) was added Fc (280.27 mg, 5.02 mmol, 5 eq) and NH₄Cl (268.45 mg, 5.02 mmol, 5 eq), the reaction mixture was stirred at 80° C. for 1 hr. The reaction mixture was quenched with water (5 mL), filtered and the filter cake washed with EA (3 mL), the filtrates were extracted with EA (3 mL*3), combined the organic phase dried by anhydrous Na₂SO₄, concentrated to give I-139-6 (0.3 g. 725.95 μmol, 72.33% yield, 94% purity) was obtained as black brown oil. LCMS (Method E): R^{t}=0.560 min, (M+H)=389.2. ¹H NMR (400 MHZ, DMSO-d₆) δ=7.62 (d, J=2.4 Hz, 1H), 7.42-7.29 (m, 3H), 7.16 (d, J=2.4 Hz, 2H), 4.98 (s, 2H), 4.44-4.31 (m, 2H), 3.77 (t, J=4.8 Hz, 2H), 3.64-3.62 (m, 2H), 3.60-3.55 (m, 5H), 3.54-3.50 (m, 2H), 2.65 (q, J=7.6 Hz, 2H), 2.54 (t, J=6.4 Hz, 2H), 1.21 (t. J=7.6 Hz, 3H).

### Step 4: Synthesis of I-139-8

To a solution of I-139-6 (0.3 g, 772.29 μmol, 1 eq) in DCM (3 mL) was added TEA (117.22 mg, 1.16 mmol, 161.24 μL, 1.5 eq), then 2-chloroacetyl chloride (87.22 mg, 772.29 μmol, 61.51 μL, 1 eq) was added to the reaction mixture at 0° C., then stirred at 0-25° C. for 2 hr. The reaction mixture was quenched with water (5 mL) and then extracted with DCM (3 mL*3), the organic phase was dried by anhydrous Na₂SO₄, filtered to give filtrates and then concentrates to give I-139-8 (0.3 g, crude) was obtained as brown oil. LCMS (Method G): R^{t}=0.709 min, (M+H)=465.0. ¹H NMR (400 MHZ, DMSO-d₆) δ=9.68 (s, 1H), 8.60 (d, J=1.6 Hz, 1H), 8.21 (d, J=2.4 Hz, 1H), 7.44-7.36 (m, 3H), 7.23 (d, J=6.8 Hz, 1H), 4.56-4.50 (m, 2H), 4.45 (s, 2H), 3.85-3.78 (m, 2H), 3.64-3.60 (m, 2H), 3.57 (s, 5H), 3.53-3.49 (m, 2H), 2.68 (q, J=7.6 Hz, 2H), 2.54-2.52 (m, 2H), 1.22 (t, J=7.6 Hz, 3H).

### Step 5: Synthesis of I-139-10

To a mixture of I-139-8 (0.3 g, 645.25 μmol, 1 eq) in MeCN (3 mL) was added I-139-9 (115.28 mg, 645.25 μmol, 1 eq, HCl), KI (428.45 mg, 2.58 mmol, 4 eq), DIEA (333.57 mg, 2.58 mmol, 449.56 μL, 4 eq), the reaction mixture was stirred at 60° C. for 2 hrs, The reaction mixture was filtered to give filtrates. The filtrates was purified by reversed phase preparative HPLC (105 g) of SepaFlash® Spherical C18, 20-45 μm, 100 Å; mobile phase: A for H₂O+0.1% NH3·H₂O and B for acetonitrile; gradient: B 40-50% in 30 min, flow rate: 60 mL/min; column temperature: RT, wavelength: 220 nm/254 nm), after purification, then concentrated to remove organic solvents then lyophilized to give I-139-10 (0.25 g. 420.12 μmol, 65.11% yield, 95.901% purity) was obtained as brown oil. LCMS (Method G): R^{t}=0.631 min, (M+H)=571.2. ¹H NMR (400 MHZ, DMSO-d₆) δ=9.90 (br s, 1H), 8.86 (d, J=2.4 Hz, 1H), 8.13 (d, J=2.4 Hz, 1H), 7.44-7.35 (m, 3H), 7.27-7.14 (m, 1H), 4.54-4.52 (m, 2H), 3.81-3.79 (m, 2H), 3.64-3.54 (m, 7H), 3.52-3.47 (m, 2H), 3.32-3.23 (m, 6H), 2.96-2.85 (m, 1H), 2.80 (s, 3H), 2.71-2.65 (m, 2H), 2.48-2.29 (m, 3H), 1.97-1.95 (m, 3H), 1.56-1.37 (m, 1H), 1.22 (t, J=7.6 Hz, 3H).

### Step 6: Synthesis of I-139-11

To a solution of I-139-10 (250 mg, 438.08 μmol, 1 eq) in DCM (2.5 mL) was added Boc20 (95.61 mg, 438.08 μmol, 100.64 μL, 1 eq), TEA (53.19 mg, 525.69 μmol, 73.17 μL, 1.2 eq), the reaction mixture was stirred at 25° C. for 12 hrs. The reaction mixture was quenched by water (5 mL), extracted with DCM (3 mL*3), the organic phase was dried by anhydrous Na₂SO₄, filtered and the filtrates were concentrated to get I-139-11 (300 mg, crude) was obtained as yellow oil. The crude product was used into next step without any other purification. LCMS (Method E): R^{t}=0.594 min, (M+H)=617.4.

### Step 7: Synthesis of I-139-12

To a solution of I-139-11 (300 mg, crude) in THF (3 mL) and H₂O (1 mL) was added LiOH·H₂O (22.52 mg, 536.68 μmol, 1.2 eq), the reaction mixture was stirred at 25° C. for 1 hr The reaction mixture was adjust to pH=5, then extracted by EA (2 mL*2), the organic phase was dried by Na₂SO₄, filtered to give filtrates, concentrated to give I-139-12 (280 mg, crude). The crude product was used into next step directly without any other purification. LCMS (Method E): R^{t}=0.563 min, (M+H)=657.4. R^{t}=1.877 min, 2.166 min._G4. ¹H NMR (400 MHZ, DMSO-d₆) δ=9.32 (s, 1H), 8.64 (d, J=8.8 Hz, 1H), 8.15 (br s, 1H), 7.45-7.32 (m, 3H), 7.23-7.21 (m, 1H), 4.52 (t, J=4.8 Hz, 2H), 4.12-4.05 (m, 2H), 3.81 (s, 2H), 3.62-3.57 (m, 4H), 3.54-3.49 (m, 2H), 3.31-3.10 (m, 6H), 2.79 (s, 3H), 2.67 (q, J=7.4 Hz, 2H), 2.43 (t, J=6.4 Hz, 2H), 2.35-2.21 (m, 1H), 2.14-2.02 (m, 1H), 1.98-1.91 (m, 1H), 1.90-1.81 (m, 1H), 1.45-1.28 (m, 9H), 1.22 (t, J=7.4 Hz, 3H).

### Step 8: Synthesis of I-139-13

To a solution of I-139-12 (80 mg, 121.81 μmol, 1 eq) and I-139-12.1 (72.74 mg, 121.81 μmol, 1 eq) in DMF (1 mL) was added EDCI (70.05 mg, 365.43 μmol, 3 eq), HOAt (16.58 mg, 121.81 μmol, 17.04 μL, 1 eq), NMM (36.96 mg, 365.43 μmol, 40.18 μL, 3 eq), the reaction mixture was stirred at 25° C. for 1 hr. The reaction mixture was filtered to give filtrates. The filtrates was purified by reverse phase purification (45 g of SepaFlash® Spherical C18, 20-45 μm, 100 Å; mobile phase: A for H₂O+0.1% NH₃·H₂O and B for acetonitrile; gradient: B 45%-50% in 15 min, flow rate: 80 mL/min; column temperature: RT, wavelength: 220 nm/254 nm), after purification, then concentrated to remove organic solvents, the residual aqueous solution was lyophilized to give I-139-13 (70 mg, 56.64 μmol, 46.50% yield, N/A purity) was obtained as a white solid. LCMS (Method E): R^{t}=0.538 min, (M+H)=1235.8. R=1.508 min, 1.648 min._H22. ¹H NMR (400 MHZ, CHLOROFORM-d) δ=10.48-9.84 (m, 1H), 9.23-8.71 (m, 2H), 8.34-7.98 (m, 3H), 7.40-7.31 (m, 3H), 7.25 (s, 2H), 7.20 (d, J=8.4 Hz, 3H), 7.04 (d, J=3.6 Hz, 1H), 6.46 (d, J=3.2 Hz, 1H), 5.37-4.93 (m, 2H), 4.64-4.53 (m, 2H), 4.42 (d, J=13.2 Hz, 2H), 4.28-4.00 (m, 2H), 3.92-3.86 (m, 2H), 3.81 (t, J=5.8 Hz, 2H), 3.76-3.68 (m, 4H), 3.65 (d, J=13.6 Hz, 1H), 3.61-3.52 (m, 2H), 3.42-3.08 (m, 6H), 2.92 (s, 3H), 2.71 (q, J=7.6 Hz, 2H), 2.57 (t, J=6.0 Hz, 2H), 2.51-2.30 (m, 11H), 2.26-1.95 (m, 15H), 1.91-1.81 (m, 1H), 1.43 (s, 9H), 1.28 (t, J=7.6 Hz, 3H).

### Step 9: Synthesis of I-139

To a solution of (70 mg, 56.64 μmol, 1 eq) in dioxane (0.5 mL) was added HCl/dioxane (1 mL), the reaction mixture was stirred at 25° C. for 1 h. The reaction mixture was concentrated under 25° C. to give crude product. The crude product was purified by reverse phase purification (45 g of SepaFlash® Spherical C18, 20-45 μm, 100 Å; mobile phase: A for H₂O+0.1% NH3·H₂O and B for acetonitrile; gradient: B 45%-50% in 15 min, flow rate: 80 mL/min; column temperature: RT, wavelength: 220 nm/254 nm), after purification, then concentrated to remove organic solvents, the residual aqueous solution was lyophilized to give I-139 (40 mg, 38.62 μmol, 68.19% yield, 100% purity) was obtained as an off-white solid. LCMS (Method G): R^{t}=0.666 min, (M+H)=1035.7. SFC: R^{t}=0.944 min. ¹H NMR (400 MHZ, DMSO-d₆) δ=11.64 (br s, 1H), 9.89 (br s, 1H), 8.86 (d, J=2.0 Hz, 1H), 8.74 (d, J=8.0 Hz, 1H), 8.17-8.05 (m, 2H), 7.45-7.26 (m, 7H), 7.25-7.11 (m, 2H), 6.57 (d, J=1.8 Hz, 1H), 4.91-4.76 (m, 1H), 4.54 (t, J=4.8 Hz, 2H), 4.46-4.33 (m, 2H), 3.81 (t, J=4.8 Hz, 2H), 3.64-3.37 (m, 12H), 3.30-3.25 (m, 4H), 2.80 (s, 3H), 2.66 (d, J=7.6 Hz, 2H), 2.41 (d, J=11.2 Hz, 5H), 2.34-2.15 (m, 6H), 2.04-1.79 (m, 7H), 1.54-1.37 (m, 3H), 1.21 (t, J=7.6 Hz, 3H), ¹H NMR (400 MHZ, DMSO+D2O) δ=8.85-8.75 (m, 1H), 8.11-8.03 (m, 2H), 7.40-7.23 (m, 7H), 7.19 (d, J=3.6 Hz, 1H), 7.12 (d, J=3.6 Hz, 1H), 6.55 (d, J=3.6 Hz, 1H), 4.77 (d, J=4.8 Hz, 1H), 4.50 (br s, 2H), 4.43-4.28 (m, 2H), 3.88-3.82 (m, 2H), 3.61-3.34 (m, 12H), 3.31-3.21 (m, 4H), 2.78 (s, 3H), 2.66-2.58 (m, 2H), 2.48-2.35 (m, 5H), 2.30-2.08 (m, 6H), 2.00-1.75 (m, 7H), 1.52-1.32 (m, 3H), 1.17-1.15 (m, 3H).

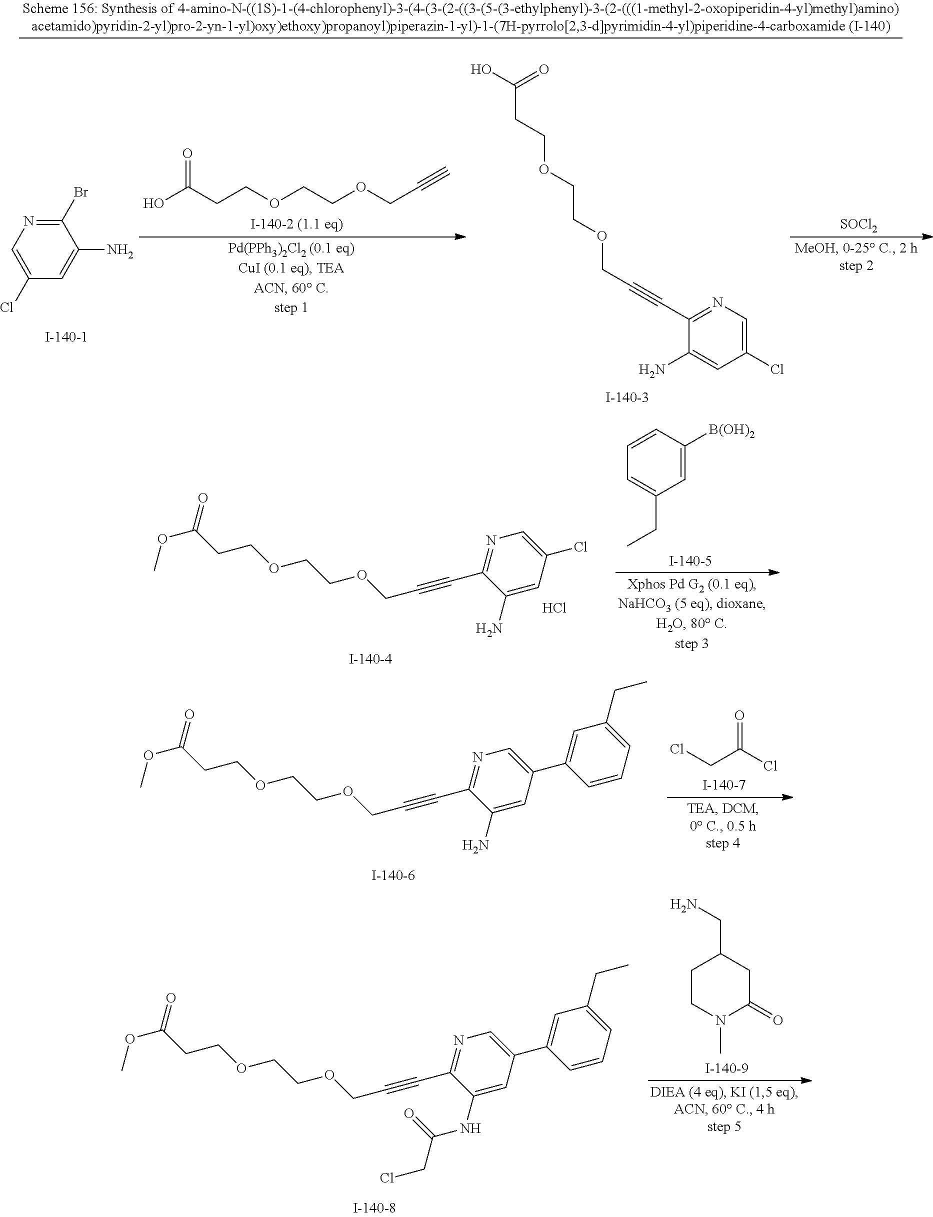

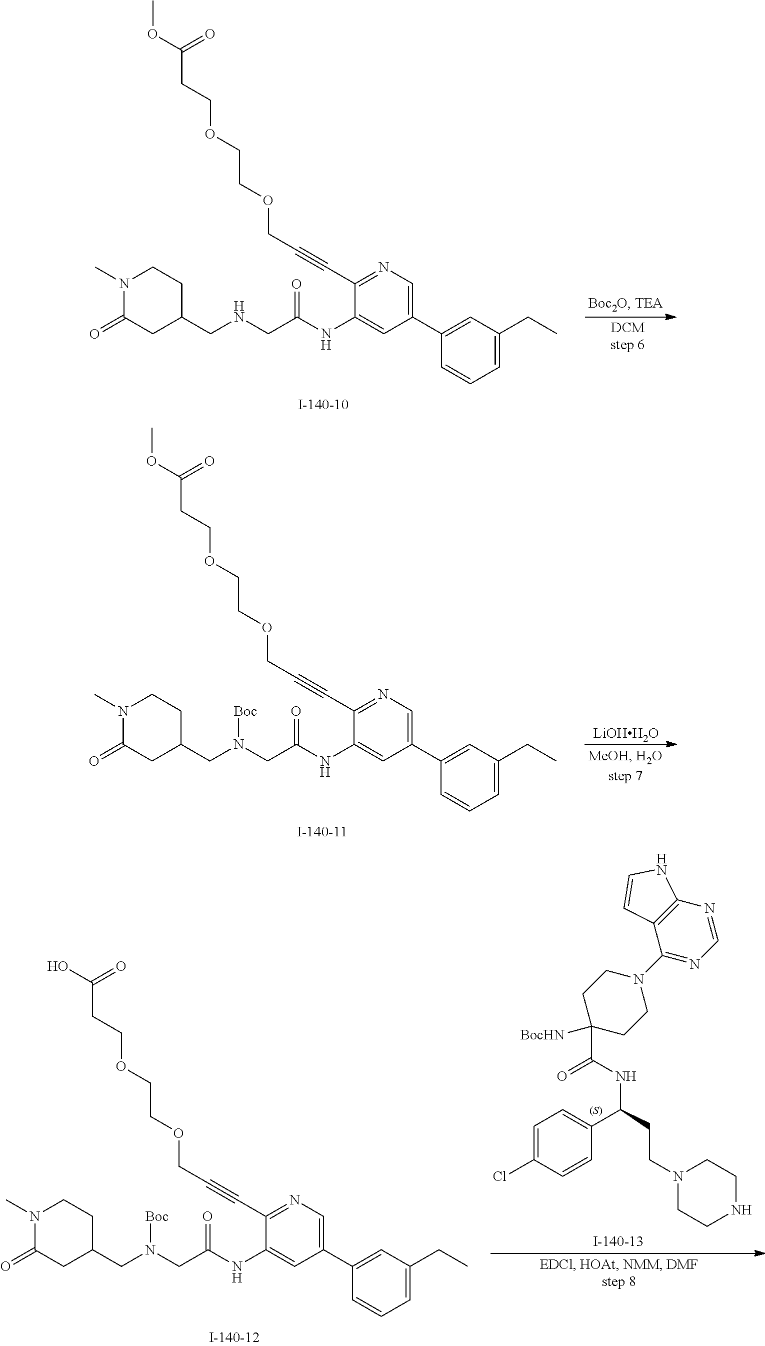

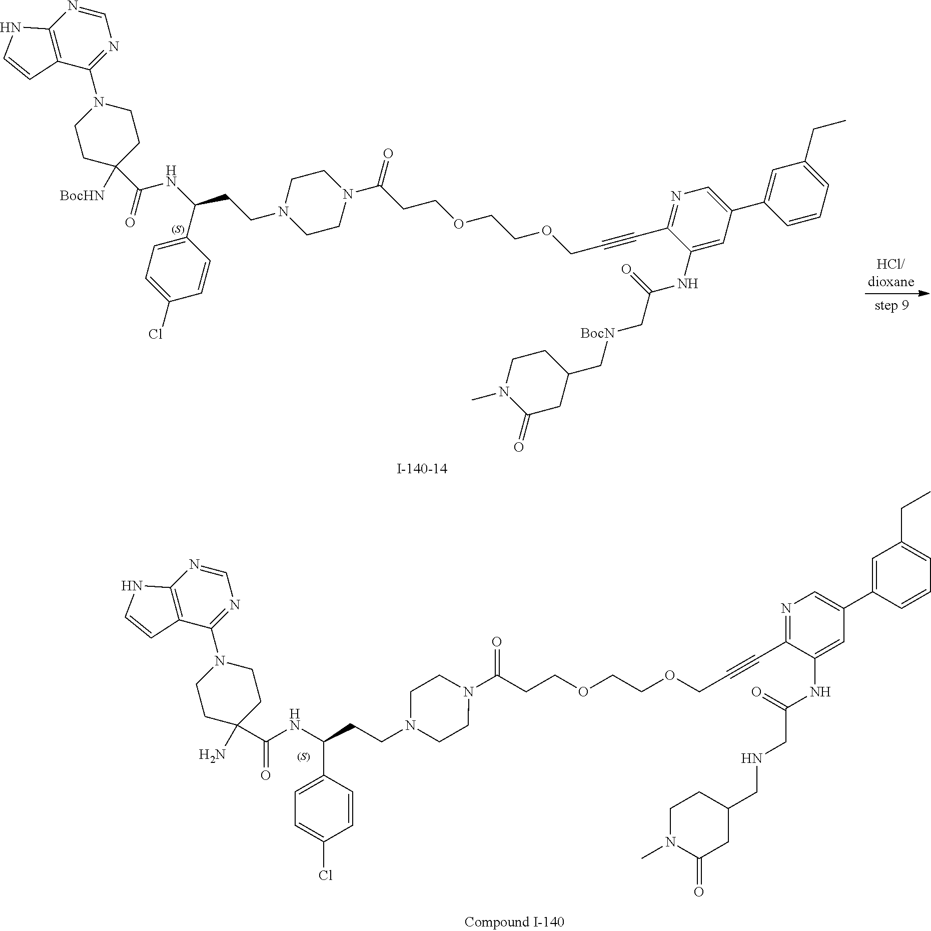

### Step 1: Synthesis of I-140-3

To a solution of I-140-1 (1.1 g. 5.30 mmol, 1 eq) and I-140-2 (1.00 g. 5.83 mmol, 1.1 eq) in ACN (15 mL) and TEA (5 mL) was added Pd(PPh₃)₂Cl₂ (372.17 mg, 530.23 μmol, 0.1 eq) under N₂. After stirred at 15° C. for 10 min, CuI (100.98 mg, 530.23 μmol, 0.1 eq) was added. The resulting solution was stirred at 60° C. for 3 hrs. The reaction mixture was concentrated under reduced pressure to give a residue. The residue was dissolved in DMSO (20 mL), filtered to give a filtrate. The filtrate was purified by reversed phase preparative HPLC (0.1% FA conditions) (300 g of XB—C18, 20-35 μm, 100 Å) Mobile phase: A for H₂O (0.1% FA v/v) and B for acetonitrile; Gradient: B 10%-75% in 20 min: Flow rate: 100 ml/min; Column temperature: R.T. Wavelength: 220 nm/254 nm). After purification, the eluent was concentrated under vacuum to remove ACN, then extracted with CHCl₃/i-PrOH (3:1, 40 mL*2). The combined organic phase was dried over Na₂SO₄, filtered and concentrated under vacuum to give I-140-3 (1.2 g, 3.75 mmol, 70.70% yield, 93.321% purity) was obtained as a brown solid. LCMS (Method B): R^{t}=0.513 min, [M+H]⁺=299.0. ¹H NMR (400 MHZ, DMSO-d₆) ¿=12.20 (s, br, 1H), 7.73 (d, J=2.1 Hz, 1H), 7.14 (d, J=2.1 Hz, 1H), 5.86 (s, 2H), 4.45 (s, 2H), 3.66-3.60 (m, 4H), 3.56 (dd, J=3.4, 5.8 Hz, 2H), 2.48-2.44 (m, 2H).

### Step 2: Synthesis of I-140-4

To a solution of I-140-3 (1.1 g. 3.68 mmol, 1 eq) in MeOH (11 mL) was added SOCl₂ (876.18 mg, 7.36 mmol, 534.91 μL, 2 eq) at 0° C. The mixture was stirred at 25° C. for 2 hrs. The mixture was concentrated under reduced pressure to get I-140-4 (1.3 g, crude, HCl) as black brown oil. It was used into the next step without further purification. LCMS (Method E): R^{t}=0.461 min, [M+H]⁺=313.3. ¹H NMR (400 MHZ, DMSO-d₆) δ=7.74 (d, J=2.4 Hz, 1H), 7.17 (d, J=2.0 Hz, 1H), 4.44 (s, 3H), 4.29 (br s, 1H), 3.66-3.61 (m, 5H), 3.57-3.53 (m, 3H), 2.58-2.52 (m, 3H).

### Step 3: Synthesis of I-140-6

To a solution of I-140-4 (1.3 g, 3.72 mmol, 1 eq, HCl) and I-140-5 (614.17 mg, 4.09 mmol, 1.1 eq) in dioxane (20 mL) and H₂O (4 mL) was added NaHCO₃ (1.56 g, 18.61 mmol, 724.25 μL, 5 eq) and [2-(2-aminophenyl)phenyl]-chloro-palladium: dicyclohexyl-[3-(2,4,6-triisopropylphenyl)phenyl]phosphane (292.90 mg, 372.27 μmol, 0.1 eq), the resulting solution was stirred at 80° C. for 4 hrs under N₂. The reaction mixture was diluted with EtOAc (20 mL), dried over Na₂SO₄, filtered and concentrated to give a residue. The residue was purified by flash silica gel chromatography (ISCO®; 40 g SepaFlash® Silica Flash Column, Eluent of 33˜50% Ethyl acetate/Petroleum ether gradient (a, 60 mL/min) to afford I-140-6 (0.51 g. 1.25 mmol, 33.66% yield, 93.960% purity) was obtained as a yellow solid. LCMS (Method E): R^{t}=0.472 min, [M+H]⁺=383.2. ¹H NMR (400 MHZ, DMSO-d₆) ¿=8.09 (d, J=2.0 Hz, 1H), 7.50-7.38 (m, 3H), 7.34 (d, J=2.0 Hz, 1H), 7.28 (d, J=7.1 Hz, 1H), 5.63 (s, 2H), 4.49 (s, 2H), 3.71-3.65 (m, 4H), 3.62 (s, 3H), 3.61-3.56 (m, 2H), 2.70 (q, J=7.6 Hz, 2H), 2.59 (t, J=6.3 Hz, 2H), 1.25 (t, J=7.6 Hz, 3H).

### Step 4: Synthesis of I-140-8

To a solution of I-140-6 (0.49 g, 1.28 mmol, 1 eq) in DCM (10 mL) was added TEA (388.93 mg, 3.84 mmol, 534.98 μL, 3 eq) and I-140-7 (289.41 mg, 2.56 mmol, 204.09 μL, 2 eq) dropwise at 0° C., and stirred at 0° C. for 0.5 h under N₂. The reaction mixture was concentrated under reduced pressure to give a residue. The residue was purified by flash silica gel chromatography (ISCO®; 40 g SepaFlash® Silica Flash Column, Eluent of 20˜60% Ethyl acetate/Petroleum ether gradient (60 mL/min), the eluent was concentrated under vacuum to give I-140-8 (0.470 g, 1.01 mmol, 78.84% yield, 98.627% purity) was obtained as a yellow solid. LCMS (Method E): R^{t}=0.576 min, [M+H]⁺=459.2. ¹H NMR (400 MHZ, DMSO-d₆) δ=9.98 (s, 1H), 8.73 (d, J=2.1 Hz, 1H), 8.43 (d, J=2.1 Hz, 1H), 7.59-7.52 (m, 2H), 7.44 (t, J=7.6 Hz, 1H), 7.32 (d, J=7.6 Hz, 1H), 4.51 (s, 2H), 4.44 (s, 2H), 3.71-3.64 (m, 4H), 3.60 (s, 3H), 3.60-3.56 (m, 2H), 2.70 (q, J=7.5 Hz, 2H), 2.57 (t, J=6.3 Hz, 2H), 1.24 (t, J=7.6 Hz, 3H).

### Step 5: Synthesis of I-140-10

To a solution of I-140-8 (0.47 g, 1.02 mmol, 1 eq) and I-140-9 (182.97 mg, 1.02 mmol, 1 eq, HCl) in ACN (8 mL) was added DIEA (529.44 mg, 4.10 mmol, 713.53 μL, 4 eq) and KI (255.01 mg, 1.54 mmol, 1.5 eq) at 20° C., then stirred at 60° C. for 4 hrs under N₂. The reaction mixture was concentrated under reduced pressure to give a residue. The residue was diluted with brine (10 mL), then extracted with CHCl₃/i-PrOH (3:1, 10 mL*2). The combined organic phase was dried over Na₂SO₄, filtered and concentrated under vacuum to give I-140-10 (0.57 g. 1.01 mmol, 98.57% yield, N/A purity) was obtained as a yellow solid. LCMS (Method E): R^{t}=0.454 min, [M+H]⁺=565.4.

### Step 6: Synthesis of I-140-11

To a solution of I-140-10 (0.57 g, 1.01 mmol, 1 eq) in DCM (10 mL) was added Boc₂O (264.37 mg, 1.21 mmol, 278.28 μL, 1.2 eq) and TEA (204.29 mg, 2.02 mmol, 281.00 μL, 2 eq), then stirred at 15-20° C. for 19 hrs. The reaction mixture was concentrated under vacuum to give a residue. The residue was purified by reversed phase preparative HPLC (0.1% NH₃·H₂O conditions) (105 g of XB—C18, 20-35 um, 100 Å) Mobile phase: A for H₂O (0.1% NH₃·H₂O v/v) and B for acetonitrile; Gradient: B 5%-90% in 25 min: Flow rate: 60 ml/min; Column temperature: R.T. Wavelength: 220 nm/254 nm). After purification, the eluent was concentrated under vacuum to give I-140-11 (0.42 g, 575.46 μmol, 57.01% yield, 91.086% purity) was obtained as a yellow solid. LCMS (Method E): R^{t}=0.585 min, [M+H]⁺=665.4. SFC: R^{t}=1.014 min, 1.224 min. _H3301. ¹H NMR (400 MHZ, CHLOROFORM-d) δ=9.00 (br s, 1H), 8.57 (s, 1H), 7.52-7.36 (m, 3H), 7.31-7.28 (m, 1H), 4.62 (br s, 2H), 4.19-3.93 (m, 2H), 3.88-3.76 (m, 4H), 3.74-3.67 (m, 5H), 3.46-3.15 (m, 4H), 2.96 (s, 3H), 2.75 (q, J=7.6 Hz, 2H), 2.64 (t, J=6.4 Hz, 2H), 2.50 (br d, J=16.9 Hz, 1H), 2.33-2.18 (m, 1H), 2.14-2.03 (m, 1H), 1.96 (br d, J=13.1 Hz, 1H), 1.55 (br s, 1H), 1.49 (br s, 9H), 1.31 (t, J=7.6 Hz, 3H).

### Step 7: Synthesis of I-140-12

To a solution of I-140-11 (0.1 g, 150.42 μmol, 1 eq) in MeOH (1 mL) was added a solution of LiOH·H₂O (12.62 mg, 300.85 μmol, 2 eq) in H₂O (0.3 mL) dropwise, and stirred at 20° C. for 2 hrs. The reaction mixture was neutralized with HCl (1M) to pH=4-5, extracted with CHCl₃/i-PrOH (3:1, 5 mL*2). The combined organic phase was dried over Na₂SO₄, filtered and concentrated under vacuum to give I-140-12 (0.097 g, crude) was obtained as a white solid. LCMS (Method E): R^{t}=0.556 min, [M+H]⁺=651.4.

### Step 8: Synthesis of I-140-14

To a solution of I-140-12 (0.097 g, 149.06 μmol, 1 eq) and I-140-13 (89.01 mg, 149.06 μmol, 1 eq) in DMF (3 mL) was added HOAt (60.86 mg, 447.17 μmol, 62.55 μL, 3 eq), EDCI (85.72 mg, 447.17 μmol, 3 eq) and NMM (90.46 mg, 894.34 μmol, 98.33 μL, 6 eq) at 20° C., and stirred at 20° C. for 16 hrs. The reaction solution was diluted with EtOAc (10 mL), washed with saturated NH₄Cl aqueous solution (5 mL) and brine (5 mL*2), dried over Na₂SO₄, filtered and concentrated under vacuum to give a residue. The residue was purified by reversed phase preparative HPLC (0.1% NH₃·H₂O conditions) (105 g of XB—C18, 20-35 μm, 100 Å) Mobile phase: A for H₂O (0.1% NH₃·H₂O v/v) and B for acetonitrile; Gradient: B 5%-80% in 25 min: Flow rate: 85 ml/min; Column temperature: R.T. Wavelength: 220 nm/254 nm). After purification, the eluent was concentrated under vacuum to give I-140-14 (0.09 g, 69.83 μmol, 46.85% yield, 95.431% purity) was obtained as a yellow solid. LCMS (Method E): R^{t}=0.534 min, [M+H]⁺=1230.6. ¹H NMR (400 MHZ, DMSO-d₆) δ=11.67 (br s, 1H), 9.56 (br d, J=1.4 Hz, 1H), 8.65 (s, 1H), 8.51 (br s, 1H), 8.30 (br d, J=7.9 Hz, 1H), 8.12 (s, 1H), 7.50 (br d, J=8.5 Hz, 2H), 7.47-7.40 (m, 1H), 7.31 (s, 5H), 7.23-7.12 (m, 2H), 6.60 (dd, J=1.5, 3.3 Hz, 1H), 4.93-4.81 (m, 1H), 4.50 (br s, 2H), 4.30-4.15 (m, 2H), 4.14-4.05 (m, 2H), 3.74-3.62 (m, 5H), 3.59 (br dd, J=3.2, 5.6 Hz, 3H), 3.48 (br s, 4H), 3.29-3.14 (m, 5H), 2.79 (s, 3H), 2.71-2.64 (m, 2H), 2.57 (s, 2H), 2.37-2.12 (m, 9H), 1.99-1.91 (m, 4H), 1.91-1.80 (m, 3H), 1.46-1.32 (m, 18H), 1.23 (t, J=7.6 Hz, 3H).

### Step 9: Synthesis of I-140

To a solution of I-140-14 (0.09 g, 73.18 μmol, 1 eq) in dioxane (1 mL) was added HCl/dioxane (1 mL), and stirred at 15-20° C. for 0.5 hr under N₂. The reaction suspension was concentrated under vacuum to give a residue. The residue was dissolved in MeOH (2 mL), neutralized with conc. NH₃·H₂O to pH=8, then purified by reversed phase preparative HPLC (0.1% NH₃·H₂O conditions) (105 g of XB—C18, 20-35 μm, 100 Å) Mobile phase: A for H₂O (0.1% NH₃·H₂O v/v) and B for acetonitrile; Gradient: B 10%-70% in 30 min; Flow rate: 85 ml/min; Column temperature: R.T. Wavelength: 220 nm/254 nm). After purification, the eluent was concentrated under vacuum to removed ACN, then it was lyophilized to give I-140 (42.41 mg, 40.76 μmol, 55.70% yield, 98.964% purity) was obtained as a white solid. LCMS (Method C): R^{t}=0.888 min, [M+H]⁺=1029.7. SFC: R^{t}=4.969 min, 5.672 min. _DN1001.1H NMR (400 MHZ, DMSO-d₆) δ=11.65 (br s, 1H), 8.95 (d, J=2.0 Hz, 1H), 8.76 (br d, J=8.1 Hz, 1H), 8.61 (d, J=2.1 Hz, 1H), 8.11 (s, 1H), 7.54-7.47 (m, 2H), 7.46-7.40 (m, 1H), 7.32 (d, J=6.8 Hz, 5H), 7.14 (dd, J=2.6, 3.4 Hz, 1H), 6.57 (dd, J=1.8, 3.6 Hz, 1H), 4.89-4.78 (m, 1H), 4.54 (s, 2H), 4.45-4.33 (m, 2H), 3.68-3.61 (m, 4H), 3.57 (dd, J=3.4, 5.8 Hz, 2H), 3.55-3.41 (m, 6H), 3.36-3.34 (m, 2H), 3.26 (dd, J=3.6, 7.9 Hz, 2H), 2.79 (s, 3H), 2.72-2.66 (m, 2H), 2.59-2.54 (m, 2H), 2.52 (br d, J=1.9 Hz, 2H), 2.47-2.27 (m, 6H), 2.26-2.13 (m, 4H), 2.05-1.72 (m, 8H), 1.51-1.36 (m, 3H), 1.22 (t, J=7.6 Hz, 3H).

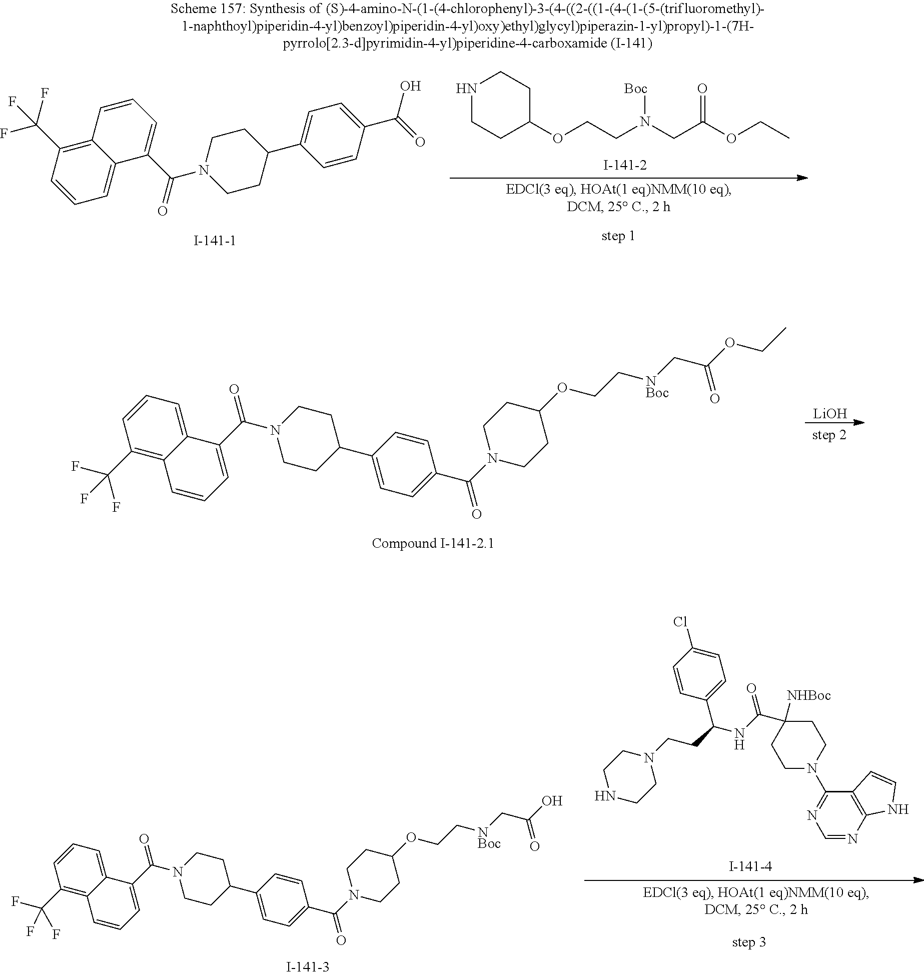 [SMILES: O=C(O)c1ccc(C2CCN(C(=O)c3cccc4c(C(F)(F)F)cccc34)CC2)cc1 ; CCOC(=O)CN(CCOC1CCNCC1)C(=O)OC(C)(C)C ; CCOC(=O)CN(CCOC1CCN(C(=O)c2ccc(C3CCN(C(=O)c4cccc5c(C(F)(F)F)cccc45)CC3)cc2)CC1)C(=O)OC(C)(C)C ; "LiOH" ; CC(C)(C)OC(=O)N(CCOC1CCN(C(=O)c2ccc(C3CCN(C(=O)c4cccc5c(C(F)(F)F)cccc45)CC3)cc2)CC1)CC(=O)O ; CC(C)(C)OC(=O)NC1(C(=O)N[C@@H](CCN2CCNCC2)c2ccc(Cl)cc2)CCN(c2ncnc3[nH]ccc23)CC1]

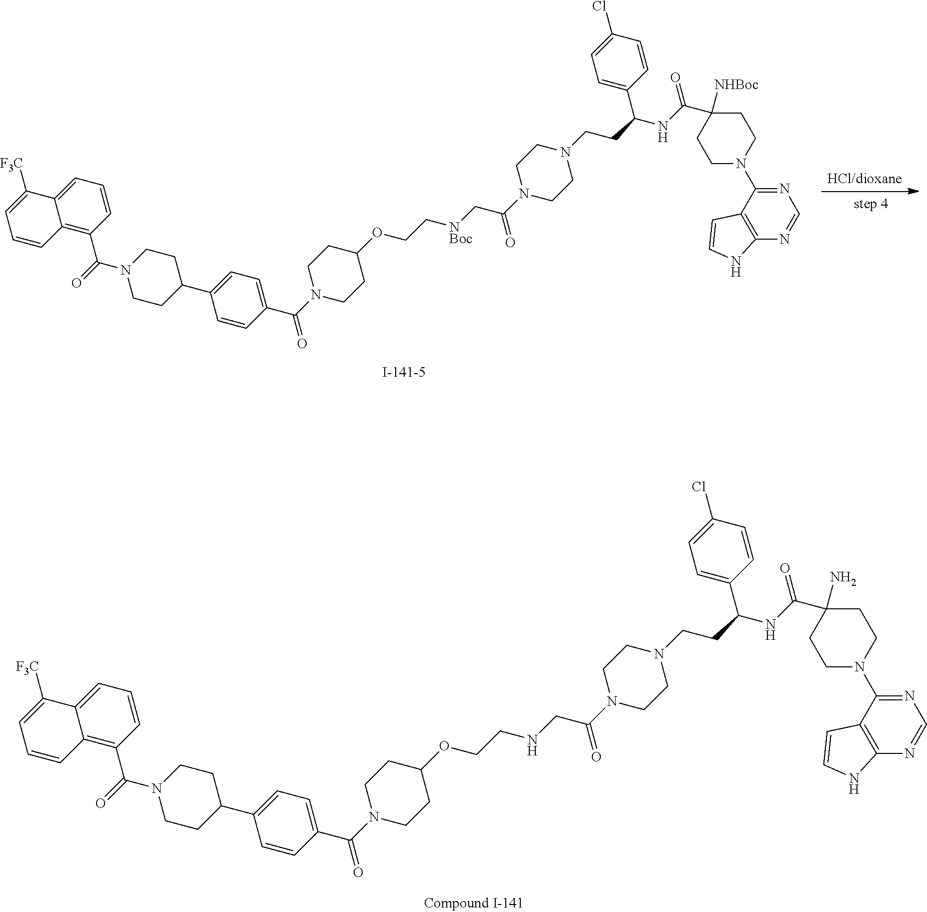 [SMILES: CC(C)(C)OC(=O)NC1(C(=O)N[C@@H](CCN2CCN(C(=O)CN(CCOC3CCN(C(=O)c4ccc(C5CCN(C(=O)c6cccc7c(C(F)(F)F)cccc67)CC5)cc4)CC3)C(=O)OC(C)(C)C)CC2)c2ccc(Cl)cc2)CCN(c2ncnc3[nH]ccc23)CC1 ; NC1(C(=O)N[C@@H](CCN2CCN(C(=O)CNCCOC3CCN(C(=O)c4ccc(C5CCN(C(=O)c6cccc7c(C(F)(F)F)cccc67)CC5)cc4)CC3)CC2)c2ccc(Cl)cc2)CCN(c2ncnc3[nH]ccc23)CC1]

### Step 1: Synthesis of I-141-2.1

To a solution of I-141-1 (100 mg, 233.96 μmol, 1 eq) and 1-2 (77.31 mg, 233.96 μmol, 1 eq) in DCM (1 mL) was added EDCI (134.55 mg, 701.89 μmol, 3 eq) HOAt (31.85 mg, 233.96 μmol, 32.73 μL, 1 eq) and NMM (236.65 mg, 2.34 mmol, 257.23 μL, 10 eq). The mixture was stirred at 25° C. for 2 h. The reaction mixture was quenched by addition H₂O 1 mL, and then diluted with DCM 1 mL and extracted with DCM 6 mL (2 mL*3). The combined organic layers were dried over anhydrous Na₂SO₄, filtered, and concentrated under reduced pressure to give a residue. The residue was purified by prep-HPLC (column: C18 150×30 mm; mobile phase: [water (FA)-ACN]; gradient: 58%-88% B over 7 min), then concentrated to remove organic solvents and lyophilized to give product. I-141-2.1 (50 mg, 67.58 μmol, 28.89% yield, 100% purity) was obtained as a white solid. LCMS (Method E): R^{t}=0.633 min, (M+H)=740.3. ¹H NMR (400 MHZ, METHANOL-d4) δ=8.29-7.99 (m, 3H), 7.80-7.56 (m, 3H), 7.43-7.29 (m, 4H), 5.05-4.98 (m, 1H), 4.23-4.13 (m, 2H), 4.06-4.00 (m, 2H), 3.99-3.87 (m, 1H), 3.67-3.36 (m, 8H), 3.30-2.86 (m, 4H), 2.14-1.48 (m, 8H), 1.47-1.38 (m, 9H), 1.33-1.22 (m, 3H).

### Step 2: Synthesis of I-141-3

To a solution of I-141-2.1 (100 mg, 135.17 μmol, 1 eq) in THF (1 mL), MeOH (1 mL) and H₂O (0.5 mL) was added LiOH·H₂O (17.02 mg, 405.50 μmol, 3 eq). The mixture was stirred at 25° C. for 1 h. The reaction mixture was concentrated under reduced pressure to give a residue and used in next step without further purification. I-141-3 (90 mg, 126.45 μmol, 93.55% yield) was obtained as a white solid. LCMS (Method E): R^{t}=0.578 min, (M+Na)=734.5.

### Step 3: Synthesis of I-141-4

To a solution of I-141-3 (90 mg, 126.45 μmol, 1 eq) and I-141-4 (83.06 mg, 139.09 μmol, 1.1 eq) in DCM (2 mL) was added EDCI (72.72 mg, 379.34 μmol, 3 eq), HOAt (17.21 mg, 126.45 μmol, 17.69 μL, 1 eq) and NMM (127.90 mg, 1.26 mmol, 139.02 μL, 10 eq). The mixture was stirred at 25° C. for 2 h. The reaction mixture was quenched by addition H₂O 1 mL, and then diluted with DCM 1 mL and extracted with DCM 6 mL (2 mL*3). The combined organic layers were dried over anhydrous Na₂SO₄, filtered and concentrated under reduced pressure to give a residue and used in next step without further purification. I-141-5 (160 mg, crude) was obtained as a yellow solid. LCMS (Method G): R^{t}=0.762 min, (M+H)=1290.7.

### Step 4: Synthesis of I-141

To a solution of I-141-5 (150 mg, 116.20 μmol, 1 eq) in DCM (1.5 mL) was added HCl/dioxane (4 M, 1.5 mL, 51.64 eq). The mixture was stirred at 25° C. for 1 h. The reaction mixture was concentrated under reduced pressure to give a residue. The residue was purified by prep-HPLC (column: Phenomenex luna C18 150*25 mm*10 um; mobile phase: [water (FA)-ACN]; gradient: 12%-42% B over min), then concentrated to remove organic solvents and lyophilized to give product. I-141 (66.69 mg, 58.67 μmol, 50.49% yield, 100% purity, FA) was obtained as a white solid. LCMS (Method E): R^{t}=0.460 min, (M+H)=1090.8. ¹H NMR (400 MHZ, METHANOL-d4) δ=8.71-8.46 (m, 1H), 8.27-8.00 (m, 4H), 7.78-7.78 (m, 1H), 7.78-7.57 (m, 3H), 7.40-7.30 (m, 8H), 7.13 (d, J=3.6 Hz, 1H), 6.62 (d, J=3.6 Hz, 1H), 5.05-4.95 (m, 2H), 4.58-4.47 (m, 2H), 4.08-3.89 (m, 3H), 3.79-3.58 (m, 8H), 3.54-3.39 (m, 4H), 3.24-3.02 (m, 4H), 2.93 (br s, 1H), 2.51-2.30 (m, 6H), 2.26-2.14 (m, 2H), 2.08-1.83 (m, 6H), 1.73-1.42 (m, 6H). SFC: R^{t}=2.331 min, ee value=100%. F2.

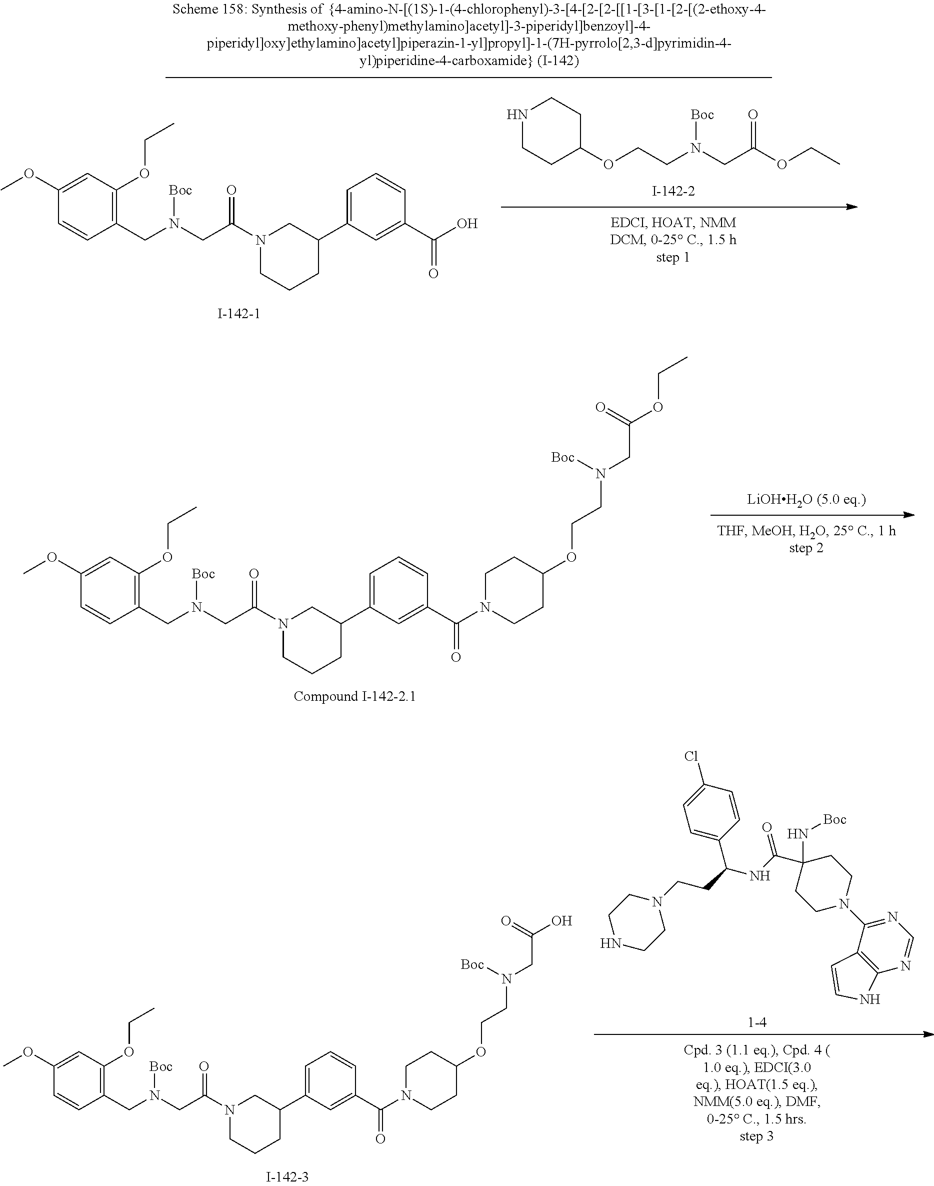

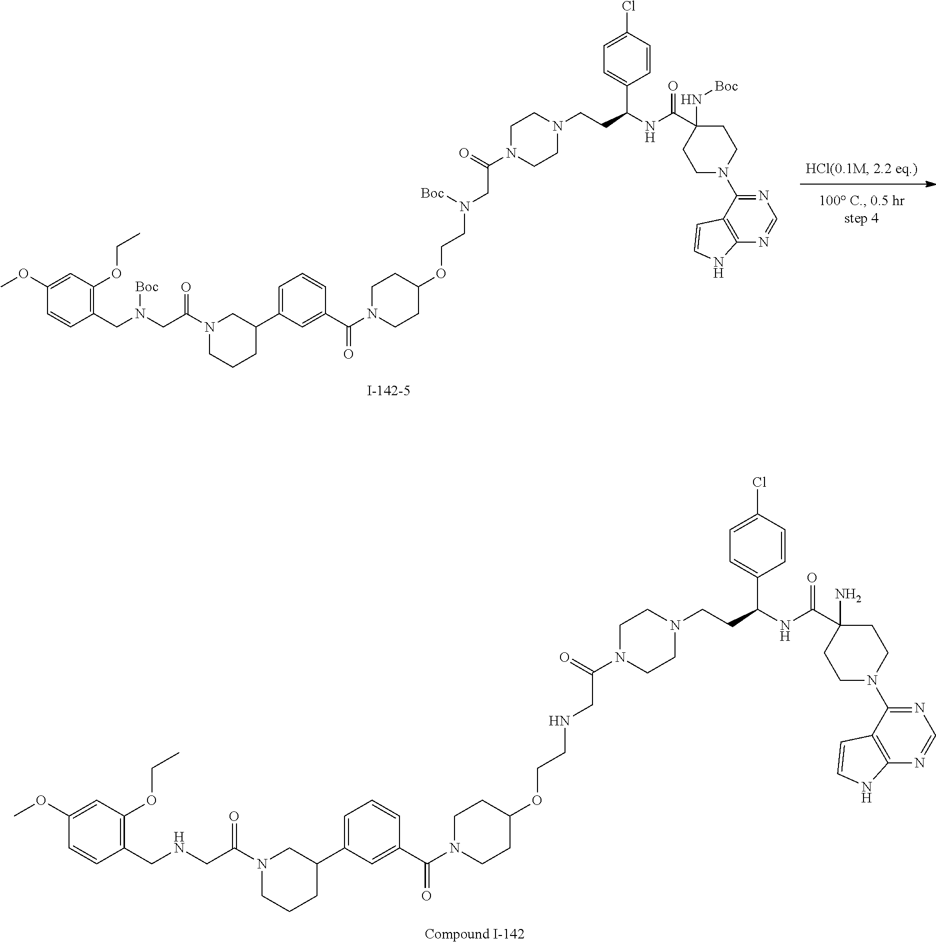 [SMILES: CCOc1cc(OC)ccc1CN(CC(=O)N1CCCC(c2cccc(C(=O)N3CCC(OCCN(CC(=O)N4CCN(CC[C@H](NC(=O)C5(NC(=O)OC(C)(C)C)CCN(c6ncnc7[nH]ccc67)CC5)c5ccc(Cl)cc5)CC4)C(=O)OC(C)(C)C)CC3)c2)C1)C(=O)OC(C)(C)C ; "HCl(0.1M, 2.2 eq.)" ; CCOc1cc(OC)ccc1CNCC(=O)N1CCCC(c2cccc(C(=O)N3CCC(OCCNCC(=O)N4CCN(CC[C@H](NC(=O)C5(N)CCN(c6ncnc7[nH]ccc67)CC5)c5ccc(Cl)cc5)CC4)CC3)c2)C1]

### Step 1: Synthesis of I-142-2.1

To a solution of 3 I-142-1 (1 g, 1.90 mmol, 1 eq) in DCM (10 mL) was added EDCI (1.09 g, 5.70 mmol, 3 eq), NMM (960.34 mg, 9.49 mmol, 1.04 mL, 5 eq), and HOAt (387.69 mg, 2.85 mmol, 398.45 μL, 1.5 eq). Then I-142-2 (690.18 mg, 2.09 mmol, 1.1 eq) was added. The reaction mixture was stirred at 25° C. for 1.5 hr. The reaction mixture was quenched by addition Na₂CO₃ 20 mL at 25° C., then extracted with EA 15 mL (5 mL*3). The combined organic layers were dried over Na₂SO₄, filtered and concentrated under reduced pressure to give a residue. The crude product was purified by reverse-phase HPLC (0.1% NH3·H₂O condition). After purification, the eluent was concentrated to remove organic solvents and then lyophilized to give product. I-142-2.1 (1.07 g. 1.28 mmol, 67.16% yield, 100% purity) as white solid. LCMS (Method G): R^{t}=0.786 min, (M+H)=839.6. ¹H NMR (400 MHZ, DMSO-d₆) δ=7.43-7.30 (m, 2H), 7.25 (br d, J=8.8 Hz, 2H), 7.06 (m, 1H), 6.58-6.41 (m, 2H), 4.48-4.17 (m, 3H), 4.16-3.84 (m, 9H), 3.72 (br d, J=8.0 Hz, 3H), 3.56-3.48 (m, 3H), 3.43-3.35 (m, 4H), 3.33-3.23 (m, 2H), 3.18-2.95 (m, 2H), 2.74-2.55 (m, 2H), 1.96-1.67 (m, 5H), 1.49-1.26 (m, 23H), 1.22-1.15 (m, 3H).

### Step 2: Synthesis of I-142-3

To a solution of I-142-2.1 (0.16 g, 190.70 μmol, 1 eq) in THF (0.3 mL), MeOH (0.3 mL), H₂O (0.3 mL) was added LiOH·H₂O (40.01 mg, 953.49 μmol, 5 eq). The mixture was stirred at 25° C. for 1 hr. The mixture was removed the ether on a rotary evaporator to give product. The crude was used into the next step without further purification. I-142-3 (0.14 g, 153.64 μmol, 80.57% yield, 89% purity) as yellow solid. LCMS (Method E): R^{t}=0.598 min, (M+H)=811.5.

### Step 3: Synthesis of I-142-5

To a solution of I-142-4 (0.1 g, 167.46 μmol, 1 eq) in DMF (1 mL) was added EDCI (96.31 mg, 502.39 μmol, 3 eq), NMM (84.69 mg, 837.31 μmol, 92.06 μL, 5 eq), and HOAt (34.19 mg, 251.19 μmol, 35.14 μL, 1.5 eq). Then I-142-3 (135.81 mg, 167.46 μmol, 1 eq) was added. The reaction mixture was stirred at 25° C. for 16 hr. The reaction mixture was quenched by addition H₂O 3 mL at 25° C., then extracted with EA 3 mL (1 mL*3). The combined organic layers were dried over Na₂SO₄, filtered, and concentrated under reduced pressure to give a residue. The crude product was purified by reverse-phase HPLC (0.1% HCl). After purification, the eluent was concentrated to remove organic solvents and then lyophilized to give product. I-142-5 (0.12 g, 64.74 μmol, 38.66% yield, 75% purity) as white solid. LCMS (Method E): R^{t}=0.550 min, (M+2)=1391.9.

### Step 4: Synthesis of I-142

A suspension of I-142-5 (0.12 g, 86.32 μmol, 1 eq) in HCl (0.1 M, 3.45 mL, 4 eq) was stirred at 100° C. for 0.5 h. The mixture was added NaHCO₃ to adjust pH=9˜10, and then lyophilized to give residue. The crude was purified by Prep-HPLC (column: Waters Xbridge 150*25 mm*5 um; mobile phase: [water (NH₃·H₂O 0.1%)-ACN]; gradient: 25%-55% B over 15 min). After prep-HPLC purification, the eluent was concentrated to remove organic solvents and then lyophilized to give product. I-142 (26.42 mg, 24.07 μmol, 27.89% yield, 99.3% purity) as white solid. LCMS (Method E): R^{t}=0.399 min, (M+H)=1089.7. SFC: R^{t}=6.699 min, 7.524 min. ¹H NMR (400 MHz, DMSO-d₆) δ=11.81-11.51 (m, 1H), 8.77 (br d, J=8.0 Hz, 1H), 8.12 (s, 1H), 7.41-7.30 (m, 6H), 7.30-7.21 (m, 2H), 7.19-7.12 (m, 2H), 6.61-6.54 (m, 1H), 6.53-6.39 (m, 2H), 4.91-4.80 (m, 1H), 4.49-4.33 (m, 3H), 4.07-3.88 (m, 3H), 3.82-3.71 (m, 4H), 3.65-3.45 (m, 10H), 3.45-3.36 (m, 5H), 3.33-3.25 (m, 2H), 3.17-2.95 (m, 2H), 2.73-2.55 (m, 4H), 2.41-2.09 (m, 10H), 1.98-1.82 (m, 6H), 1.80-1.65 (m, 3H), 1.54-1.36 (m, 5H), 1.34-1.28 (m, 3H).

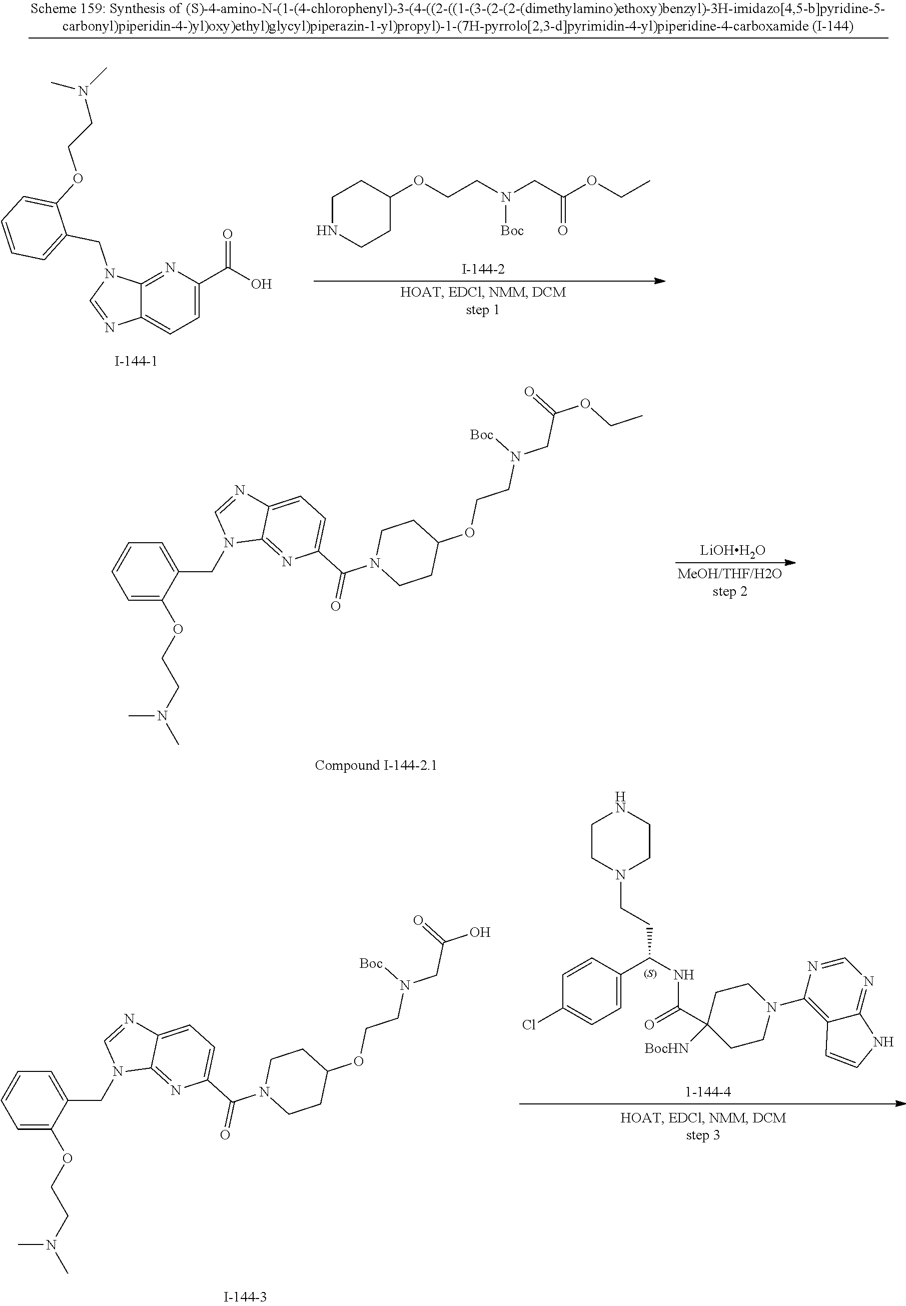

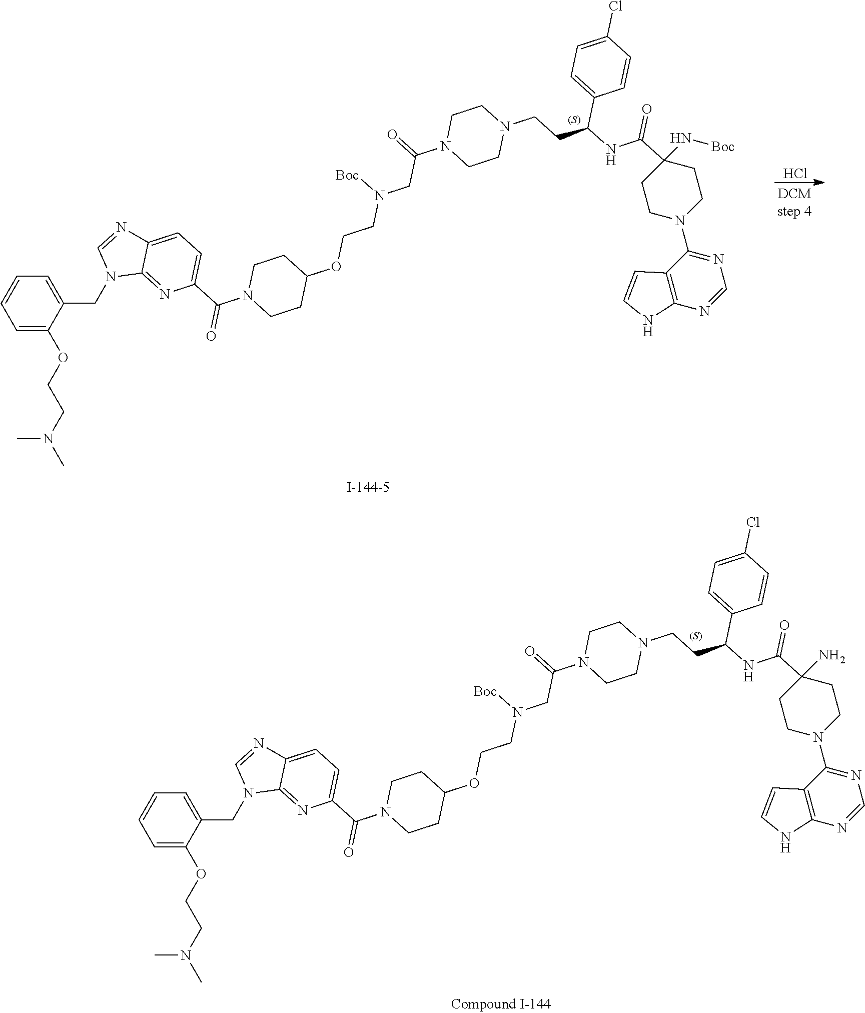

### Step 1: Synthesis of I-144-2.1

A mixture of I-144-1 (1 g, 2.94 mmol, 1 eq), I-144-2 (1.08 g, 2.94 mmol, 1 eq, HCl) and HOAt (399.88 mg, 2.94 mmol, 410.98 μL, 1 eq) in DCM (10 mL) was added EDCI (1.13 g, 5.88 mmol, 2 eq) and NMM (1.49 g, 14.69 mmol, 1.62 mL, 5 eq). The mixture was stirred at 25° C. for 12 hrs. The mixture was concentrated under vacuum. The crude product was purified by reverse-phase HPLC (0.1% NH₃·H₂O condition) and concentrated under vacuum to remove MeCN and dried by lyophilization to got 1.75 g product and 0.5 g impure product. 0.5 g crude was purified by Pre-HPLC (column: Waters Xbridge Prep OBD C18 150*40 mm*10 um; mobile phase: [water (NH4HCO3)-ACN]; gradient: 28%-58% B over 15 min) and concentrated under vacuum to get I-144-2.1 (1.85 g, 2.78 mmol, 94.77% yield, 98.248% purity) as yellow oil was obtained. LCMS (Method G): R^{t}=0.655 min, (M+H)+=653.3. ¹H NMR (400 MHZ, CHLOROFORM-d) δ=8.54 (s, 1H), 8.06 (d, J=8.0 Hz, 1H), 7.53 (d, J=8.4 Hz, 1H), 7.36 (d, J=7.6 Hz, 1H), 7.29-7.23 (m, 1H), 6.91-6.83 (m, 2H), 5.44 (s, 2H), 4.17-4.15 (m, 2H), 4.12-4.01 (m, 4H), 3.99 (s, 1H), 3.72-3.71 (m, 1H), 3.64-3.54 (m, 4H), 3.52-3.42 (m, 2H), 3.38-3.27 (m, 1H), 2.74-2.72 (m, 2H), 2.33 (s, 6H), 2.01-1.90 (m, 1H), 1.85-1.76 (m, 1H), 1.71 (br d, J=4.0 Hz, 1H), 1.62-1.53 (m, 1H), 1.46-1.40 (m, 9H), 1.27-1.22 (m, 3H).

### Step 2: Synthesis of I-144-3

To a solution of I-144-2.1 (0.1 g, 153.19 μmol, 1 eq) in THF (0.5 mL)/EtOH (0.5 mL)/H2O (0.5 mL) was added LiOH·H₂O (32.14 mg, 765.96 μmol, 5 eq). The mixture was stirred at 25° C. for 2 hr. The reaction mixture was adjusted to PH=5 with 2N HCl, then extracted with CHCl₃/i-PrOH=3:1 (5 mL*3). The organic layer was dried with anhydrous Na₂SO₄, filtered and concentrated under vacuum to get I-144-3 (0.1 g, crude) was obtained as a white solid. LCMS (Method E): R^{t}=0.426 min, (M+H)+=625.5.

### Step 3: Synthesis of I-144-5

To a solution of I-144-3 (0.1 g, 160.07 μmol, 1 eq) in DCM (1 mL) was added I-144-4 (114.70 mg, 192.08 μmol, 1.2 eq) HOAt (21.79 mg, 160.07 μmol, 22.39 μL, 1 eq) and EDCI (30.69 mg, 160.07 μmol, 1 eq) NMM (80.95 mg, 800.35 μmol, 87.99 μL, 5 eq). The mixture was stirred at 25° C. for 1 hr. The reaction mixture was added H₂O (2 mL) and extracted with DCM (3 mL*3) and washed with brine (2 mL*4). The organic layer was dried with anhydrous Na₂SO₄, filtered and concentrated under vacuum to get the residue. The residue was purified by prep-HPLC (FA condition) and lyophilization to get I-144-5 (0.2 g, 109.65 μmol, 68.50% yield, 66% purity) was obtained as yellow oil. LCMS (Method E): R^{t}=0.439 min, (M+H)+=1203.8.

### Step 4: Synthesis of I-144

A mixture of I-144-5 (200 mg, 166.13 μmol, 1 eq) in DCM (2 mL) was added HCl/dioxane (4 M, 1 mL, 24.08 eq). The mixture was stirred at 25° C. for 1 hr. The mixture was concentrated under vacuum. The residue was purified by Pre-HPLC (column: Phenomenex luna C18 150*25 mm*10 um; mobile phase: [water (FA)-ACN]; gradient: 0%-28% B over 9 min) and dried by lyophilization to get I-144 (54.52 mg, 52.02 μmol, 31.31% yield, 95.752% purity) as white solid was obtained. LCMS (Method G): R^{t}=0.583 min, (M+H)+=1003.6. SFC: R^{t}=1.644 min. F2. ¹H NMR (400 MHZ, DMSO-d₆) δ=11.66 (br s, 1H), 8.72 (br d, J=7.6 Hz, 1H), 8.68 (s, 1H), 8.16 (s, 2H), 8.11 (s, 1H), 7.45 (d, J=8.4 Hz, 1H), 7.38-7.30 (m, 4H), 7.30-7.23 (m, 1H), 7.18-7.09 (m, 2H), 7.04 (d, J=8.0 Hz, 1H), 6.87-6.86 (m, 1H), 6.58 (d, J=2.0 Hz, 1H), 5.43 (s, 2H), 4.90-4.79 (m, 1H), 4.45-4.35 (m, 2H), 4.11-4.08 (m, 2H), 4.02-3.91 (m, 2H), 3.57 (br s, 3H), 3.56-3.54 (m, 5H), 3.20-3.15 (m, 3H), 2.80-2.79 (m, 2H), 2.70-2.67 (m, 2H), 2.39-2.27 (m, 4H), 2.26 (s, 6H), 2.21-2.20 (m, 3H), 2.05-1.79 (m, 6H), 1.77-1.65 (m, 1H), 1.60-1.33 (m, 5H).

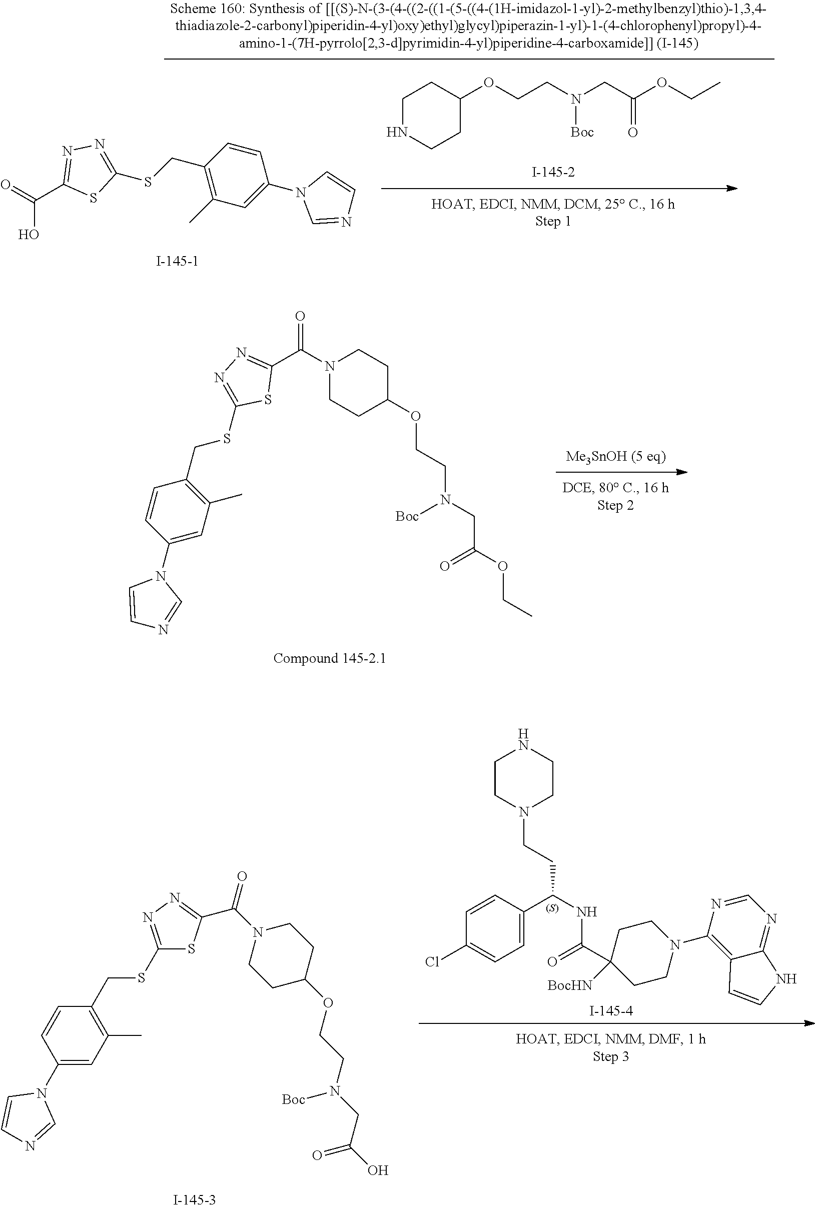

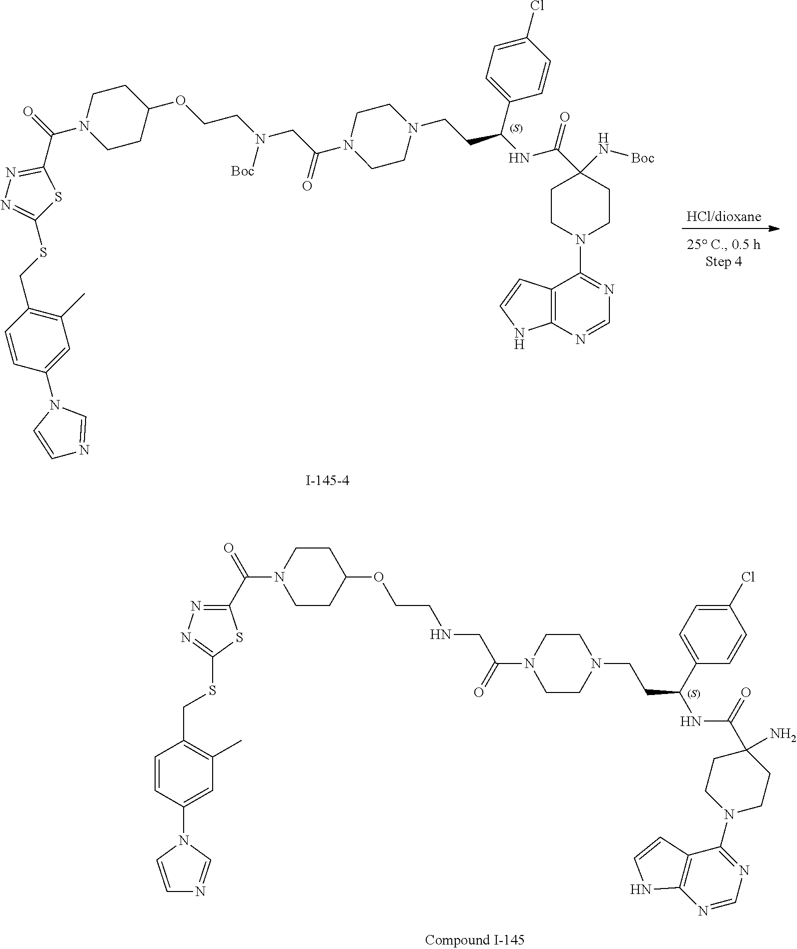

### Step 1: Synthesis of I-145.2.1

To a solution of I-145-1 (1 g, 2.81 mmol, 1 eq, sodium salt), I-145-2 (1.02 g, 3.10 mmol, 1.1 eq), HOAt (574.48 mg, 4.22 mmol, 590.42 μL, 1.5 eq), EDCI (1.62 g, 8.44 mmol, 3 eq) in DCM (10 mL) was added NMM (1.42 g, 14.07 mmol, 1.55 mL, 5 eq), then the mixture was stirred at 25° C. for 12 hr. The reaction mixture was quenched with water (10 mL) and then extracted with DCM (5 mL*3), the organic phase was dried by anhydrous Na₂SO₄, filtered to give filtrates and then concentrates to give crude product. The crude product was purified by reverse phase purification (80 g of SepaFlash® Spherical C18, 20-45 μm, 100 Å; mobile phase: A for H₂O+0.1% NH3·H₂O and B for acetonitrile; gradient: B 40%-65% in 30 min, flow rate: 150 mL/min; column temperature: RT, wavelength: 220 nm/254 nm), after purification, then concentrated to remove organic solvents, the residual aqueous solution was lyophilized to give product. I-145-2.1 (1.32 g. 2.02 mmol, 71.89% yield, 98.817% purity) was obtained as yellow gum. LCMS (Method G): R^{t}=0.703 min, (M+H)=645.1. ¹H NMR (400 MHZ, DMSO-d₆) δ=8.24 (s, 1H), 7.73 (s, 1H), 7.56 (d, J=8.4 Hz, 2H), 7.46-7.44 (m, 1H), 7.09 (s, 1H), 4.69 (s, 2H), 4.21 (d, J=4.4 Hz, 1H), 4.15-4.05 (m, 2H), 3.97 (d, J=7.4 Hz, 2H), 3.90-3.73 (m, 2H), 3.64-3.49 (m, 3H), 3.48-3.34 (m, 3H), 2.46 (s, 3H), 1.85 (d, J=1.4 Hz, 2H), 1.51 (d, J=4.0 Hz, 2H), 1.42-1.32 (m, 9H), 1.22-1.15 (m, 3H).

### Step 2: Synthesis of I-145-3

To a solution of I-145-2.1 (200 mg, 310.17 μmol, 1 eq) in DCE (2 mL) was added hydroxy (trimethyl) stannane (280.43 mg, 1.55 mmol, 5 eq) and then the reaction mixture was stirred at 80° C. for 6 hr. The reaction mixture was filtered, and the filter cake was washed with DCM (0.5 mL*3), the filtrates was concentrated to give crude product. The crude product was purified by flash silica gel chromatography (ISCO®; 10 g SepaFlash® Silica Flash Column, Eluent of 0˜40% MeOH/DCM @ 60 mL/min). After purification, then concentrated to give product. I-145-3 (190 mg, 308.07 μmol, 99.32% yield, N/A purity) was obtained as white solid. LCMS (Method G): R^{t}=0.389 min, (M+H)=617.1. ¹H NMR (400 MHZ, DMSO-d₆) δ=8.24 (s, 1H), 7.73 (t, J=1.2 Hz, 1H), 7.60-7.53 (m, 2H), 7.46-7.40 (m, 1H), 7.09 (s, 1H), 4.69 (s, 2H), 4.30-4.12 (m, 1H), 3.93-3.72 (m, 4H), 3.64-3.37 (m, 6H), 2.46 (s, 3H), 1.86 (d, J=6.0 Hz, 2H), 1.59-1.45 (m, 2H), 1.37 (d, J=19.6 Hz, 9H).

### Step 3: Synthesis of I-145-5

To a solution of I-145-3 (0.09 g, 145.93 μmol, 1 eq) and I-145-4 (87.14 mg, 145.93 μmol, 1 eq), EDCI (83.92 mg, 437.78 μmol, 3 eq) and NMM (73.80 mg, 729.63 μmol, 80.22 μL, 5 eq) in DMF (0.9 mL) was added HOAt (19.86 mg, 145.93 μmol, 20.41 μL, 1 eq) and then the mixture was stirred at 25° C. for 1 hr. The reaction mixture was filtered to give filtrates. The filtrate was purified by reversed phase preparative HPLC (45 g) of SepaFlash® Spherical C18, 20-45 μm, 100 Å; mobile phase: A for H₂O+0.1% NH₃·H₂O and B for acetonitrile; gradient: B 50-60% in 30 min, flow rate: 60 mL/min; column temperature: RT, wavelength: 220 nm/254 nm), after purification, then concentrated to remove organic solvents then lyophilized to give product. I-145-5 (0.09 g, 75.26 μmol, 51.57% yield) was obtained as an off-white solid. LCMS (Method G): R^{t}=0.724 min, (1/2M+H)=598.3. R^{t}=2.226 min. F3.1H NMR (400 MHz, DMSO-d₆) δ=11.65 (br s, 1H), 8.24 (s, 1H), 8.12 (s, 1H), 7.73 (s, 1H), 7.55 (d, J=8.0 Hz, 2H), 7.46-7.44 (m, 1H), 7.32 (s, 4H), 7.22-7.05 (m, 3H), 6.59 (br s, 1H), 4.90-4.82 (m, 1H), 4.68 (s, 2H), 4.27-4.14 (m, 3H), 4.07-4.05 (m, 2H), 3.90-3.74 (m, 2H), 3.65-3.34 (m, 11H), 2.46 (s, 3H), 2.43-2.17 (m, 7H), 2.04-1.75 (m, 9H), 1.58-1.45 (m, 3H), 1.44-1.30 (m, 16H).

### Step 4: Synthesis of I-145

To a solution of I-145-5 (0.09 g, 75.26 μmol, 1 eq) in dioxane (0.5 mL) was added HCl/dioxane (4 M, 0.5 mL, 26.58 eq) and then the mixture was stirred at 25° C. for 0.5 hr The reaction mixture was concentrated under room temperature reduced pressure to remove solvent to give a residue. The residue was purified by reversed phase preparative HPLC (45 g) of SepaFlash® Spherical C18, 20-45 μm, 100 Å; mobile phase: A for H₂O+0.1% NH3·H₂O and B for acetonitrile; gradient: B 40-50% in 30 min, flow rate: 60 mL/min; column temperature: RT, wavelength: 220 nm/254 nm), after purification, then concentrated to remove organic solvents then lyophilized to give product. I-145 (32.13 mg, 30.85 μmol, 41.00% yield, 95.606% purity) was obtained as a white solid. LCMS (Method G): R^{t}=0.604 min, (M+H)=995.7. R^{t}=1.581 min. F1. ¹H NMR (400 MHZ, DMSO-d₆) δ=11.65 (br s, 1H), 8.75 (d, J=8.8 Hz, 1H), 8.25 (s, 1H), 8.11 (s, 1H), 7.74 (s, 1H), 7.61-7.52 (m, 2H), 7.50-7.42 (m, 1H), 7.40-7.24 (m, 4H), 7.20-7.05 (m, 2H), 6.57 (br s, 1H), 4.91-4.76 (m, 1H), 4.68 (s, 2H), 4.45-4.30 (m, 2H), 4.27-4.18 (m, 1H), 3.91-3.71 (m, 2H), 3.64-3.34 (m, 16H), 2.46 (s, 3H), 2.36-2.23 (m, 5H), 2.00-1.82 (m, 6H), 1.59-1.48 (m, 2H), 1.47-1.36 (m, 2H).

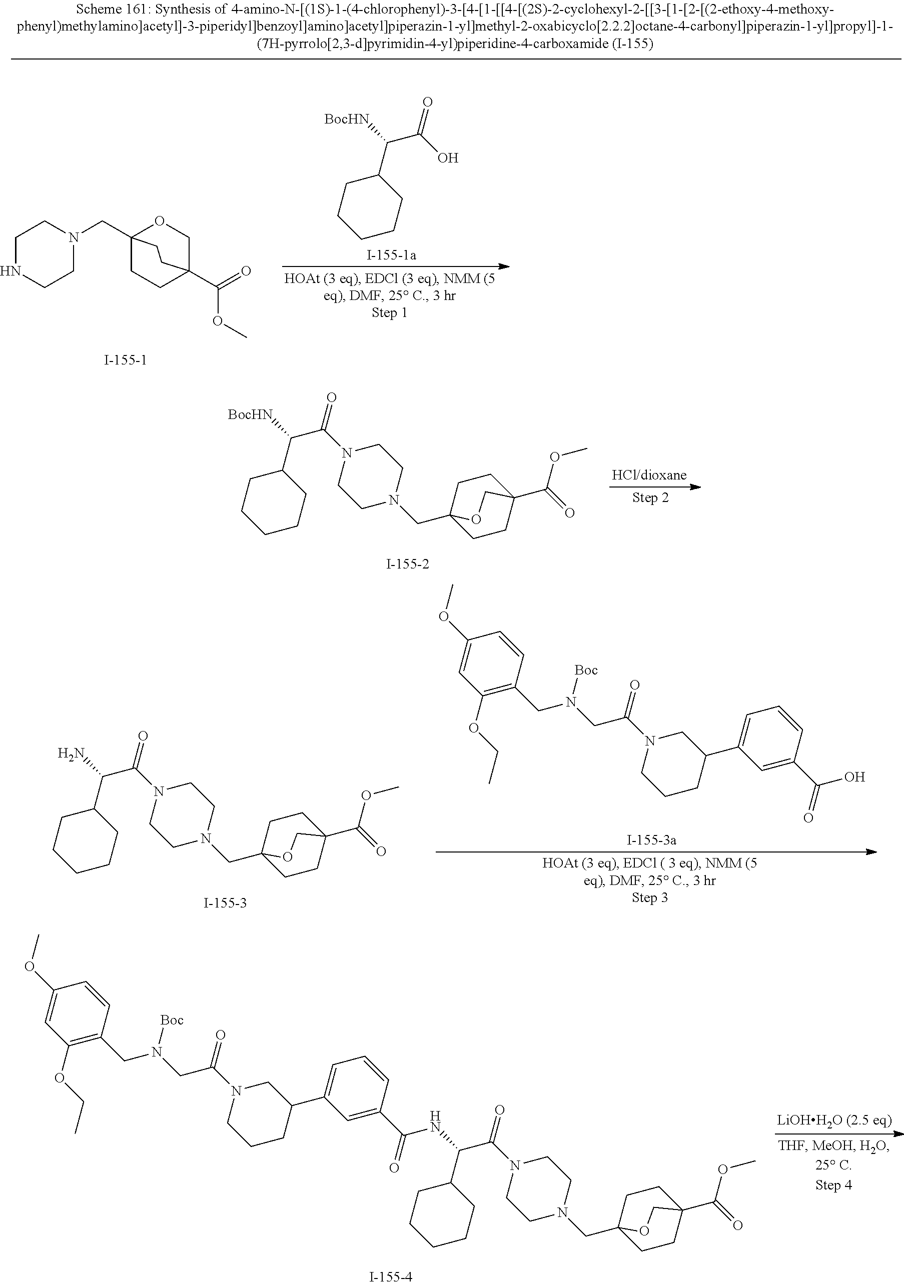 [SMILES: COC(=O)C12CCC(CN3CCNCC3)(CC1)OC2 ; CC(C)(C)OC(=O)N[C@H](C(=O)O)C1CCCCC1 ; COC(=O)C12CCC(CN3CCN(C(=O)[C@@H](NC(=O)OC(C)(C)C)C4CCCCC4)CC3)(CC1)OC2 ; COC(=O)C12CCC(CN3CCN(C(=O)[C@@H](N)C4CCCCC4)CC3)(CC1)OC2 ; CCOc1cc(OC)ccc1CN(CC(=O)N1CCCC(c2cccc(C(=O)O)c2)C1)C(=O)OC(C)(C)C ; CCOc1cc(OC)ccc1CN(CC(=O)N1CCCC(c2cccc(C(=O)N[C@H](C(=O)N3CCN(CC45CCC(C(=O)OC)(CC4)CO5)CC3)C3CCCCC3)c2)C1)C(=O)OC(C)(C)C]

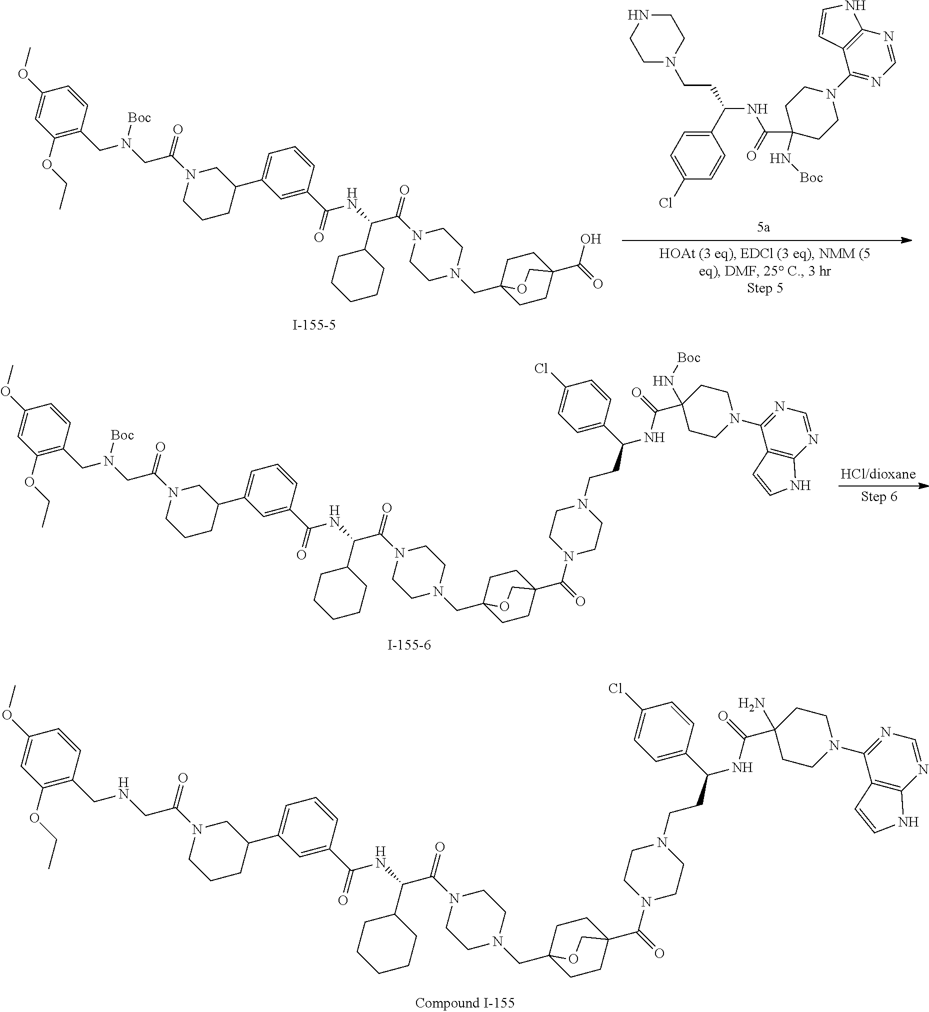

### Step 1: Synthesis of I-155-2

To a solution of I-155-2 (400 mg, 1.49 mmol, 1 eq.) in DMF (5 mL) was added EDCI (857.24 mg, 4.47 mmol, 3 eq.) and HOAt (608.66 mg, 4.47 mmol, 625.54 μL, 3 eq.), NMM (753.84 mg, 7.45 mmol, 819.39 μL, 5 eq.), I-155-1a (575.35 mg, 2.24 mmol, 1.5 eq.). The mixture was stirred at 25° C. for 3 hr. The reaction mixture was filtered and diluted with H₂O (25 mL) and extracted with EA (25 mL*3). The combined organic layers were washed with brine (20 mL*2), dried over anhydrous Na₂SO₄, filtered and concentrated under reduced pressure to give a residue. The crude product was purified by reverse-phase HPLC (column: Phenomenex luna C18 150*25 mm*10 um; mobile phase: [water-ACN]; gradient: 48%-78% B over min) to give I-155-2 (370 mg, 683.28 μmol, 45.84% yield, 93.750% purity, R^{t}=1.122 min, ee=100%) as white solid. LCMS (Method C): R^{t}=1.003 min, (M+H)=508.2.

### Step 2: Synthesis of I-155-3

To a solution of I-155-2 (200 mg, 393.96 μmol, 1 eq.) in dioxane (1 mL) was added HCl/dioxane (4 M, 1 mL, 10.15 eq.). The mixture was stirred at 25° C. for 2 hr. The reaction mixture was filtered and concentrated under reduced pressure to give I-155-3 (250 mg, crude, HCl) as yellow oil. LCMS (Method C): R^{t}=0.776 min, [M+H]⁺=408.3.

### Step 3: Synthesis of I-155-4

To a solution of I-155-3 (189.70 mg, 427.25 μmol, 1.5 eq., HCl) in DMF (2 mL) was added EDCI (163.81 mg, 854.51 μmol, 3 eq.) and HOAt (116.31 mg, 854.51 μmol, 119.54 μL, 3 eq.), NMM (144.05 mg, 1.42 mmol, 156.58 μL, 5 eq.), I-155-3a (150 mg, 284.84 μmol, 1 eq.). The mixture was stirred at 25° C. for 3 hr. The reaction mixture was filtered and diluted with H₂O (10 mL) and extracted with EA (20 mL*3). The combined organic layers were washed with brine (15 mL*2), dried over anhydrous Na₂SO₄, filtered and concentrated under reduced pressure to give I-155-4 (250 mg, 240.17 μmol, 84.32% yield, 88.011% purity) as yellow oil w. LCMS (Method E): R^{t}=0.561 min, [M+H]⁺=916.6.

### Step 4: Synthesis of I-155-5

To a solution of I-155-4 (260 mg, 283.80 μmol, 1 eq.) in THF (0.3 mL), MeOH (0.3 mL), H₂O (0.3 mL) was added LiOH·H₂O (29.77 mg, 709.49 μmol, 2.5 eq.). The mixture was stirred at 25° C. for 4 hr The mixture was concentrated under reduced pressure to remove THF and MeOH, then diluted with H₂O (10 mL), pH of the mixture was adjusted to 3 by 1N HCl and extracted with EA (20*3 mL), the organic layers was washed with brine (10*3 ml), dried over Na₂SO₄, filtered and concentrated under reduced pressure to give I-155-5 (200 mg, 183.41 μmol, 64.63% yield, 82.729% purity) as white solid. LCMS (Method E): R^{t}=0.540 min, [M+H]⁺=902.4.

### Step 5: Synthesis of I-155-6

To a solution of I-155-5 (100 mg, 110.85 μmol, 1 eq.) in DMF (0.5 mL) was added EDCI (63.75 mg, 332.55 μmol, 3 eq.) and HOAT (45.26 mg, 332.55 μmol, 46.52 μL, 3 eq.), NMM (56.06 mg, 554.25 μmol, 60.94 μL, 5 eq.), I-155-5a (79.43 mg, 133.02 μmol, 1.2 eq.). The mixture was stirred at 25° C. for 3 hr. The reaction mixture was filtered and diluted with H₂O (5 mL) and extracted with EA (20 mL*3). The combined organic layers were washed with brine (20 mL*2), dried over anhydrous Na₂SO₄, filtered and concentrated under reduced pressure to give I-155-6 (100 mg, crude) as white solid. LCMS (Method E): R^{t}=0.527 min, [M+H]⁺=1480.8.

### Step 6: Synthesis of I-155

To a solution of I-155-6 (100 mg, 67.51 μmol, 1 eq.) in dioxane (0.5 mL) was added HCl/dioxane (4 M, 0.5 mL, 29.63 eq.). The mixture was stirred at 25° C. for 3 hr. The mixture was concentrated under reduced pressure to remove dioxane, then diluted with H₂O (10 mL), pH of the mixture was adjusted to 8 by NaHCO₃ and extracted with EA (3*20 mL), the organic layers was washed with brine (10 ml*2), dried over Na₂SO₄, filtered and concentrated under reduced pressure. The residue was purified by prep-HPLC (column: Phenomenex luna C18 150*25 mm*10 um; mobile phase: [water (TFA)-ACN]; gradient: 16%-46% B over min) to give I-155 (23.15 mg, 18.07 μmol, 26.77% yield, 100% purity) as white solid. R^{t}=0.442 min, [M+H]⁺=1280.8. ¹H NMR (400 MHZ, METHANOL-d4) δ=8.34 (d, J=1.6 Hz, 1H), 7.80-7.71 (m, 2H), 7.54-7.42 (m, 2H), 7.38-7.26 (m, 6H), 6.87 (t, J=3.0 Hz, 1H), 6.56 (br s, 2H), 5.07-4.97 (m, 1H), 4.70-4.50 (m, 4H), 4.26-4.10 (m, 6H), 4.09-3.93 (m, 6H), 3.81 (d, J=3.6 Hz, 7H), 3.75-3.65 (m, 2H), 3.62-3.39 (m, 4H), 3.28-3.18 (m, 6H), 3.16-3.06 (m, 2H), 2.97-2.69 (m, 3H), 2.67-2.51 (m, 2H), 2.42-2.08 (m, 6H), 2.07-1.85 (m, 10H), 1.85-1.75 (m, 5H), 1.74-1.50 (m, 4H), 1.50-1.40 (m, 3H), 1.39-1.04 (m, 6H).

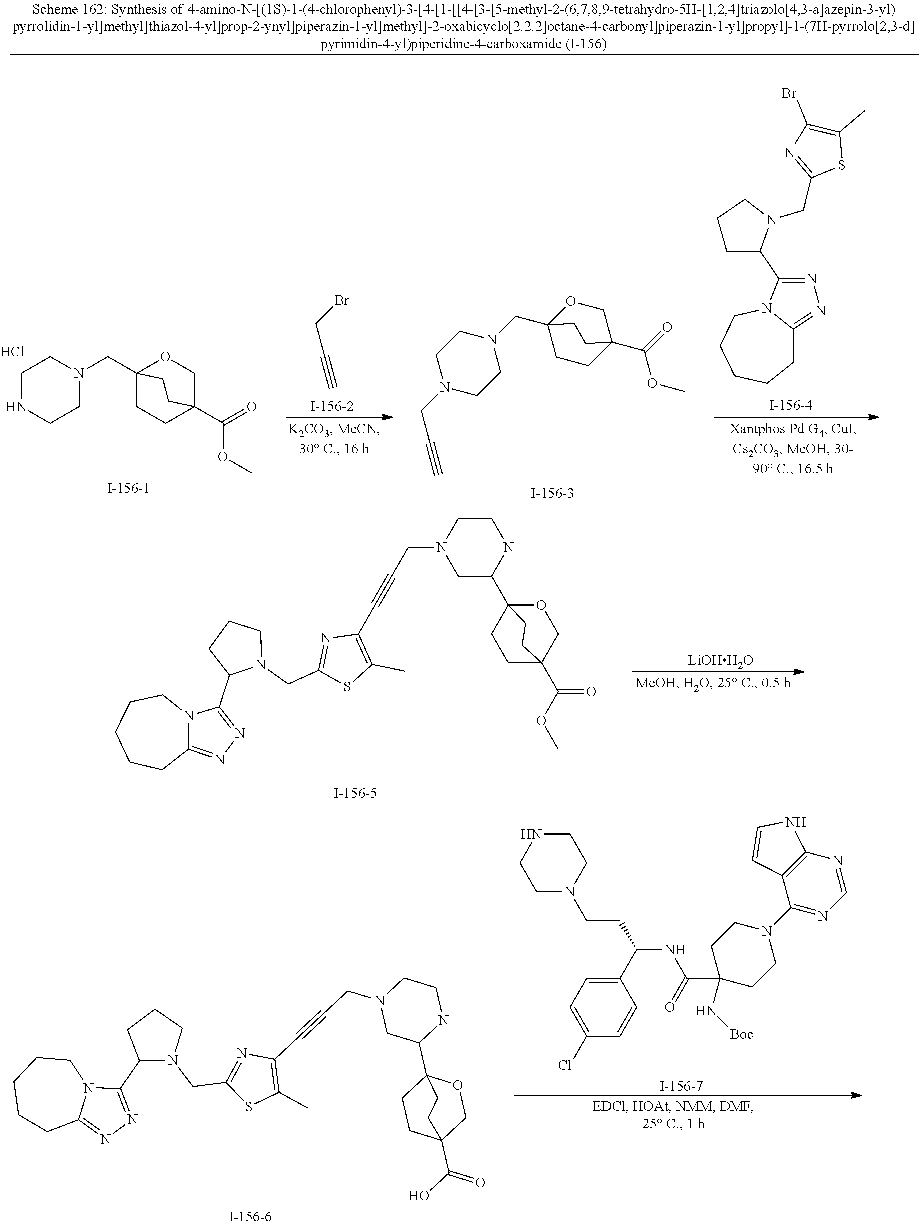

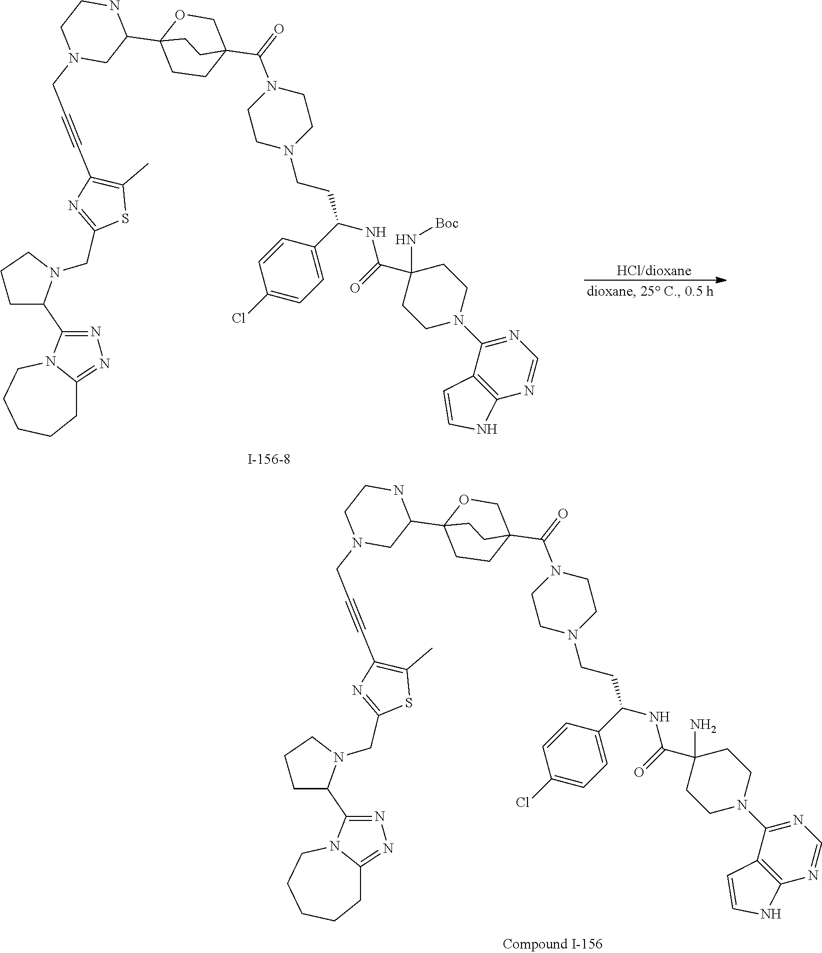 [SMILES: Cc1sc(CN2CCCC2c2nnc3n2CCCCC3)nc1C#CCN1CCNC(C23CCC(C(=O)N4CCN(CC[C@H](NC(=O)C5(NC(=O)OC(C)(C)C)CCN(c6ncnc7[nH]ccc67)CC5)c5ccc(Cl)cc5)CC4)(CC2)CO3)C1 ; "HCl/dioxane" ; Cc1sc(CN2CCCC2c2nnc3n2CCCCC3)nc1C#CCN1CCNC(C23CCC(C(=O)N4CCN(CC[C@H](NC(=O)C5(N)CCN(c6ncnc7[nH]ccc67)CC5)c5ccc(Cl)cc5)CC4)(CC2)CO3)C1]

### Step 1: Synthesis of I-156-3

To a solution of I-156-1 (300 mg, 984.21 μmol, 1 eq, HCl) in MeCN (3 mL) was added K₂CO₃ (408.07 mg, 2.95 mmol, 3 eq) and I-156-2 (117.08 mg, 787.37 μmol, 84.84 μL, 0.8 eq). The mixture was stirred at 30° C. for 16 hr. The reaction mixture was filtered and the filtrate was concentrated. The residue was purified by flash silica gel chromatography (ISCO®; 20 g SepaFlash® Silica Flash Column, Eluent of 0˜30% MeOH/DCM gradient @ 40 mL/min). I-156-3 (120 mg, 368.15 μmol, 37.41% yield, 94% purity) was obtained. LCMS (Method G): R^{t}=0.503 min, [M+H]⁺=307.2. ¹H NMR (400 MHZ, METHANOL-d4) δ=3.90 (s, 2H), 3.64 (s, 3H), 3.27 (d, J=2.4 Hz, 2H), 2.68 (t, J=2.4 Hz, 1H), 2.59 (br s, 8H), 2.27 (s, 2H), 1.98-1.89 (m, 6H), 1.70-1.56 (m, 2H).

### Step 2: Synthesis of I-156-5

To a solution of I-156-4 (115 mg, 290.15 μmol, 1 eq) in MeCN (2 mL) was added Cs₂CO₃ (283.61 mg, 870.45 μmol, 3 eq), CuI (552.59 μg, 2.90 μmol, 0.01 eq) and Xantphos Pd G4 (27.92 mg, 29.01 μmol, 0.1 eq). The mixture was purged with N₂ for three times and stirred at 30° C. for 0.5 h under N₂ atmosphere. Then I-156-3 (88.90 mg, 290.15 μmol, 1 eq) was added and the mixture was heated to 90° C., and stirred at 90° C. for 16 h under N₂ atmosphere. The mixture was concentrated. The crude product was purified by reverse-phase HPLC (0.1% NH₃·H₂O:ACN: 0-50%). I-156-5 (120 mg, 150.52 μmol, 51.88% yield, 78% purity) was obtained as a yellow oil. LCMS (Method G): R^{t}=0.607 min, [M+H]⁺=622.2.

### Step 3: Synthesis of I-156-6

To a solution of I-156-5 (120 mg, 150.52 μmol, 1 eq) in MeOH (1.5 mL) was added LiOH H₂O (31.58 mg, 752.61 μmol, 5 eq) in H₂O (0.5 mL). The mixture was adjusted to pH=3 by HCl and extracted with DCM (2 mL*3). The combined organic layers were dried over Na₂SO₄, filtered and concentrated. The crude was purified by Prep-HPLC (column: C18 150×30 mm; mobile phase: [water (FA)-ACN]; gradient: 12%-42% B over 7 min). I-156-6 (45 mg, 66.63 μmol, 44.27% yield, 90% purity) was obtained as a yellow oil. LCMS (Method E): R^{t}=0.386 min, [M+H]⁺=608.5. ¹H NMR (400 MHZ, METHANOL-d4) δ=8.53 (s, 3H), 4.36 (br s, 2H), 4.05-3.96 (m, 1H), 3.95-3.86 (m, 4H), 3.80-3.68 (m, 1H), 3.58 (s, 2H), 3.30-3.23 (m, 1H), 2.95 (br dd, J=3.6, 6.8 Hz, 2H), 2.72 (br s, 9H), 2.48 (s, 4H), 2.39 (s, 2H), 2.35-2.24 (m, 1H), 2.09-1.98 (m, 3H), 1.84-1.52 (m, 7H).

### Step 4: Synthesis of I-156-8

To a solution of I-156-6 (25 mg, 41.13 μmol, 1 eq) in DMF (0.5 mL) was added EDCI (23.65 mg, 123.39 μmol, 3 eq), HOAt (16.80 mg, 123.39 μmol, 17.26 μL, 3 eq) and NMM (20.80 mg, 205.66 μmol, 22.61 μL, 5 eq) and I-156-7 (36.84 mg, 61.70 μmol, 1.5 eq). The mixture was stirred at 25° C. for 1 h. The mixture was washed with water (3 mL) and extracted with DCM (2 mL*3). The combined organic layers were dried over Na₂SO₄, filtered and concentrated. I-156-8 (85 mg, 36.52 μmol, 88.79% yield, 51% purity) was obtained as a yellow oil. LCMS (Method C): R^{t}=0.974 min, [M+H]⁺=1186.8.

### Step 5: Synthesis of I-156

To a solution of I-156-8 (85 mg, 36.52 μmol, 1 eq) in dioxane (0.25 mL) was added HCl/dioxane (0.25 mL) (4 M). The mixture was stirred at 25° C. for 0.5 h. The mixture was concentrated. The crude was purified by Prep-HPLC (column: C18 150×30 mm; mobile phase: [water (FA)-ACN]; gradient: 0%-30% B over 12 min). I-156 (11.58 mg, 10.10 μmol, 27.66% yield, 98% purity; HCl salt) was obtained as a yellow solid. LCMS (Method C): R^{t}=0.895 min, [M+H]⁺=1086.7. ¹H NMR (400 MHZ, METHANOL-d4) δ=8.56-8.31 (m, 1H), 8.15 (s, 1H), 7.38-7.25 (m, 4H), 7.14 (d, J=3.6 Hz, 1H), 6.64 (d, J=3.6 Hz, 1H), 4.99 (br t, J=6.4 Hz, 1H), 4.67-4.49 (m, 2H), 4.46-4.26 (m, 2H), 4.00 (br t, J=8.0 Hz, 1H), 3.96-3.86 (m, 3H), 3.79-3.53 (m, 9H), 3.29-3.23 (m, 1H), 3.00-2.69 (m, 10H), 2.60-2.10 (m, 18H), 2.08-1.79 (m, 13H), 1.76-1.62 (m, 6H). SFC: Method details: “Column: Chiralpak AD-3 50*4.6 mm I.D., 3 um; Mobile phase: Phase A for CO₂, and Phase B for IPA+CAN (0.05% DEA); Gradient elution: 50% IPA+CAN (0.05% DEA) in CO₂; Flow rate: 3 mL/min; Detector: PDA; Column Temp: 35° C.; R^{t}=3.812 min, 5.168 min; H1302.

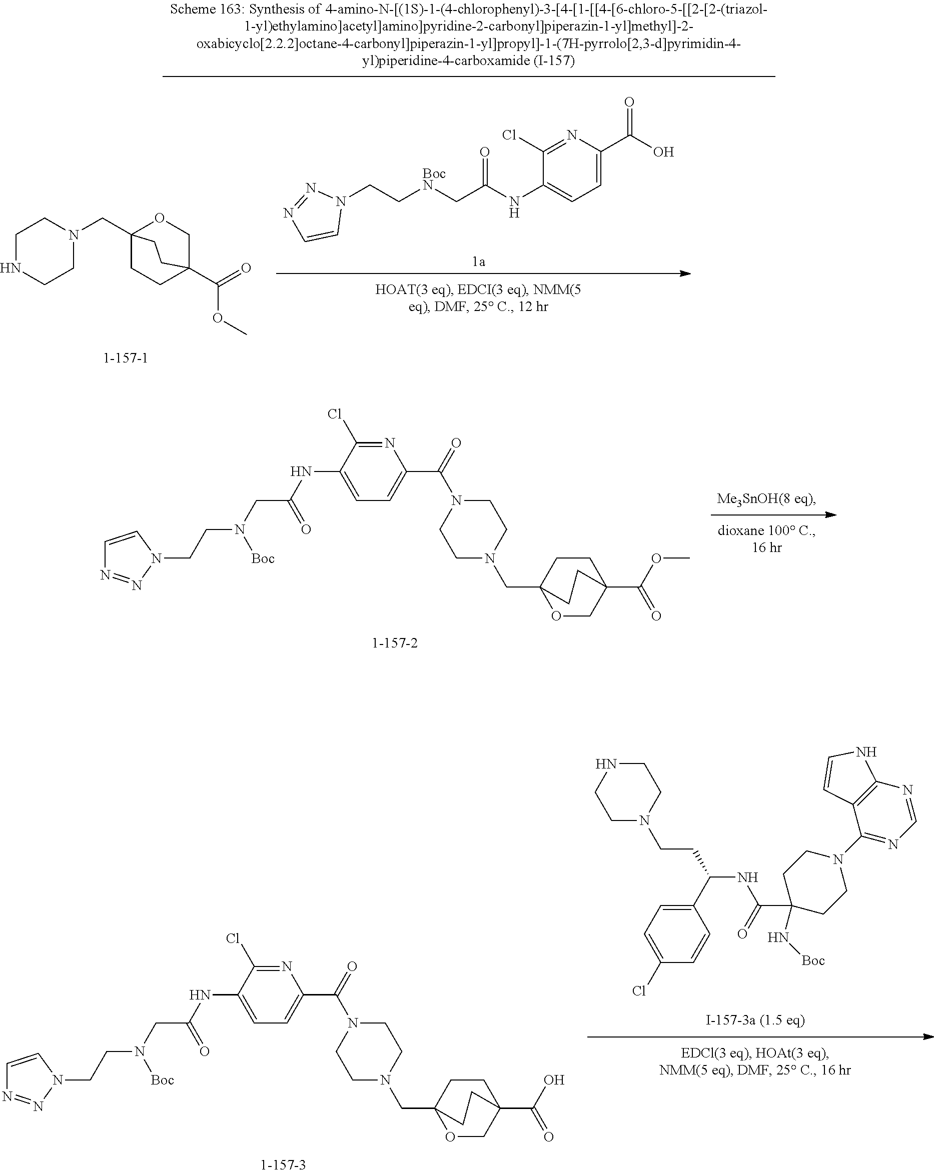 [SMILES: COC(=O)C12CCC(CN3CCNCC3)(CC1)OC2 ; CC(C)(C)OC(=O)N(CCn1ccnn1)CC(=O)Nc1ccc(C(=O)O)nc1Cl ; "1a" ; COC(=O)C12CCC(CN3CCN(C(=O)c4ccc(NC(=O)CN(CCn5ccnn5)C(=O)OC(C)(C)C)c(Cl)n4)CC3)(CC1)OC2 ; CC(C)(C)OC(=O)N(CCn1ccnn1)CC(=O)Nc1ccc(C(=O)N2CCN(CC34CCC(C(=O)O)(CC3)CO4)CC2)nc1Cl ; CC(C)(C)OC(=O)NC1(C(=O)N[C@@H](CCN2CCNCC2)c2ccc(Cl)cc2)CCN(c2ncnc3[nH]ccc23)CC1]

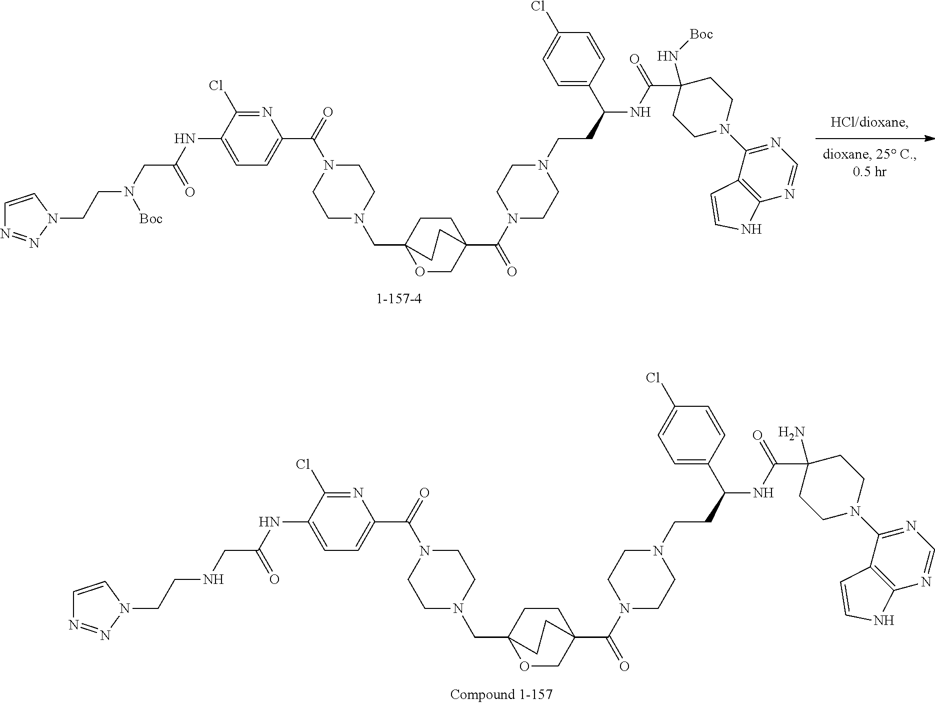 [SMILES: CC(C)(C)OC(=O)NC1(C(=O)N[C@@H](CCN2CCN(C(=O)C34CCC(CN5CCN(C(=O)c6ccc(NC(=O)CN(CCn7ccnn7)C(=O)OC(C)(C)C)c(Cl)n6)CC5)(CC3)OC4)CC2)c2ccc(Cl)cc2)CCN(c2ncnc3[nH]ccc23)CC1 ; NC1(C(=O)N[C@@H](CCN2CCN(C(=O)C34CCC(CN5CCN(C(=O)c6ccc(NC(=O)CNCCn7ccnn7)c(Cl)n6)CC5)(CC3)OC4)CC2)c2ccc(Cl)cc2)CCN(c2ncnc3[nH]ccc23)CC1]

### Step 1: Synthesis of I-157-2

To a solution of I-157-1a (200 mg, 470.77 μmol, 1 eq), EDCI (270.74 mg, 1.41 mmol, 3 eq) and HOAt (192.23 mg, 1.41 mmol, 3 eq) in DMF (2 mL) was added I-157-1 (189.50 mg, 706.15 μmol, 1.5 eq) and NMM (238.08 mg, 2.35 mmol, 258.79 μL, 5 eq), the mixture was stirred at 25° C. for 2 hr. The residue was diluted with H₂O (10 mL) and was extracted with ethyl acetate (5 mL*3). The combined organic phase was washed with brine (10 mL*1), dried with anhydrous Na₂SO₄, filtered and concentrated in vacuum. The crude product was purified by reverse-phase HPLC (0.1% FA condition). I-157-2 (200 mg, 292.67 μmol, 62.17% yield, 98.8% purity) was obtained as yellow oil. LCMS (Method G): R^{t}=0.575 min, [M+H]⁺=675.3.

### Step 2: Synthesis of I-157-3

To a solution of methyl I-157-2 (200 mg, 296.22 μmol, 1 eq) in dioxane (3 mL) was added hydroxy (trimethyl) stannane (428.50 mg, 2.37 mmol, 8 eq), the mixture was stirred at 100° C. for 16 hr. The resulting product was filtered to remove the insoluble. The filter liquor was concentrated in vacuum. The resulting product was filtered to remove the insoluble. The filter liquor was concentrated in vacuum. I-157-3 (40 mg, 59.77 μmol, 20.18% yield, 98.8% purity) as yellow solid. LCMS (Method E): R^{t}=0.396 min, [M+H]⁺=661.4. ( )

### Step 3: Synthesis of I-157-4

To a solution of I-157-3 (35 mg, 52.94 μmol, 1 eq), EDCI (30.44 mg, 158.81 μmol, 3 eq) and HOAt (21.62 mg, 158.81 μmol, 22.22 μL, 3 eq) in DMF (1 mL) was added I-157-3a (47.42 mg, 79.41 μmol, 1.5 eq) and NMM (26.77 mg, 264.69 μmol, 29.10 μL, 5 eq), the mixture was stirred at 25° C. for 16 hr. The residue was diluted with H₂O (10 mL) and was extracted with ethyl acetate (5 mL*2). The combined organic phase was washed with brine (10 mL*1), dried with anhydrous Na₂SO₄, filtered and concentrated in vacuum. The crude product was purified by reverse-phase HPLC (0.1% FA condition) to give I-157-4 (100 mg, 49.18 μmol, 92.91% yield, 61% purity) as yellow oil. LCMS (Method G): R^{t}=0.652 min, [M+H]⁺=1239.7.

### Step 4: Synthesis of I-157

To a solution of I-157-4 (100 mg, 80.63 μmol, 1 eq) in DCM (1 mL) was added HCl/dioxane (1 mL). The mixture was stirred at 25° C. for 1 hr. The reaction mixture was concentrated under reduced pressure to give the crude product. The crude product was purified by Prep-HPLC (column: Phenomenex Luna C18 150*25 mm*10 um; mobile phase: [water (NH4HCO3)-ACN]; gradient: 24%-54% B over 10 min) to give I-157 (5.7 mg, 5.19 μmol, 6.44% yield, 98% purity, HCl) as white solid. LCMS (Method G): R^{t}=0.565 min, [M+H]⁺=1039.6. ¹H NMR (400 MHZ, METHANOL-d4) δ=8.76 (d, J=8.4 Hz, 1H), 8.12 (s, 1H), 8.05 (d, J=0.8 Hz, 1H), 7.74 (d, J=0.8 Hz, 1H), 7.59 (d, J=8.4 Hz, 1H), 7.32 (s, 4H), 7.11 (d, J=3.6 Hz, 1H), 6.61 (d, J=3.6 Hz, 1H), 4.97 (t, J=6.4 Hz, 1H), 4.61 (t, J=6.0 Hz, 2H), 4.53-4.45 (m, 2H), 3.93 (s, 2H), 3.82-3.57 (m, 9H), 3.54 (br s, 2H), 3.46 (s, 2H), 3.23 (t, J=6.0 Hz, 2H), 2.67-2.54 (m, 4H), 2.49-2.28 (m, 9H), 2.18 (m, 4H), 2.08-1.86 (m, 6H), 1.69-1.53 (m, 4H). SFC: R^{t}=2.664 min.

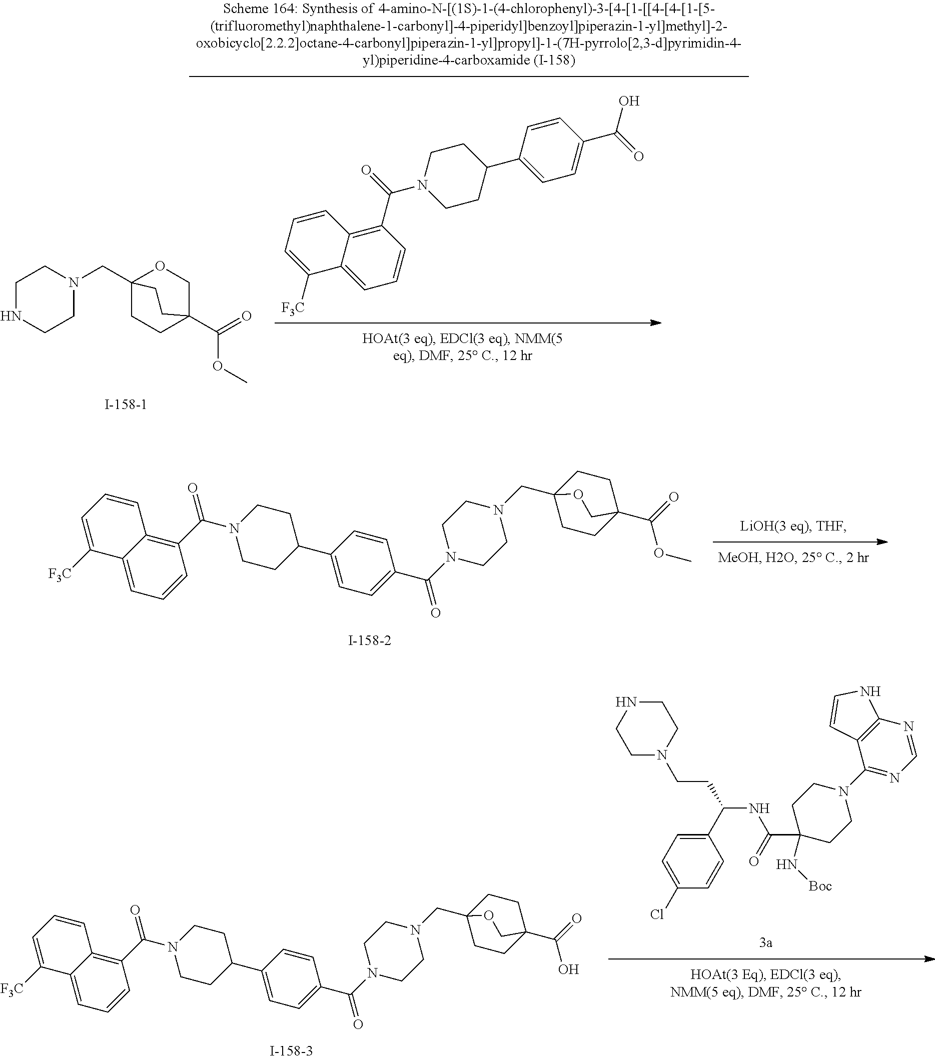 [SMILES: COC(=O)C12CCC(CN3CCNCC3)(CC1)OC2 ; O=C(O)c1ccc(C2CCN(C(=O)c3cccc4c(C(F)(F)F)cccc34)CC2)cc1 ; COC(=O)C12CCC(CN3CCN(C(=O)c4ccc(C5CCN(C(=O)c6cccc7c(C(F)(F)F)cccc67)CC5)cc4)CC3)(CC1)OC2 ; O=C(c1ccc(C2CCN(C(=O)c3cccc4c(C(F)(F)F)cccc34)CC2)cc1)N1CCN(CC23CCC(C(=O)O)(CC2)CO3)CC1 ; CC(C)(C)OC(=O)NC1(C(=O)N[C@@H](CCN2CCNCC2)c2ccc(Cl)cc2)CCN(c2ncnc3[nH]ccc23)CC1]

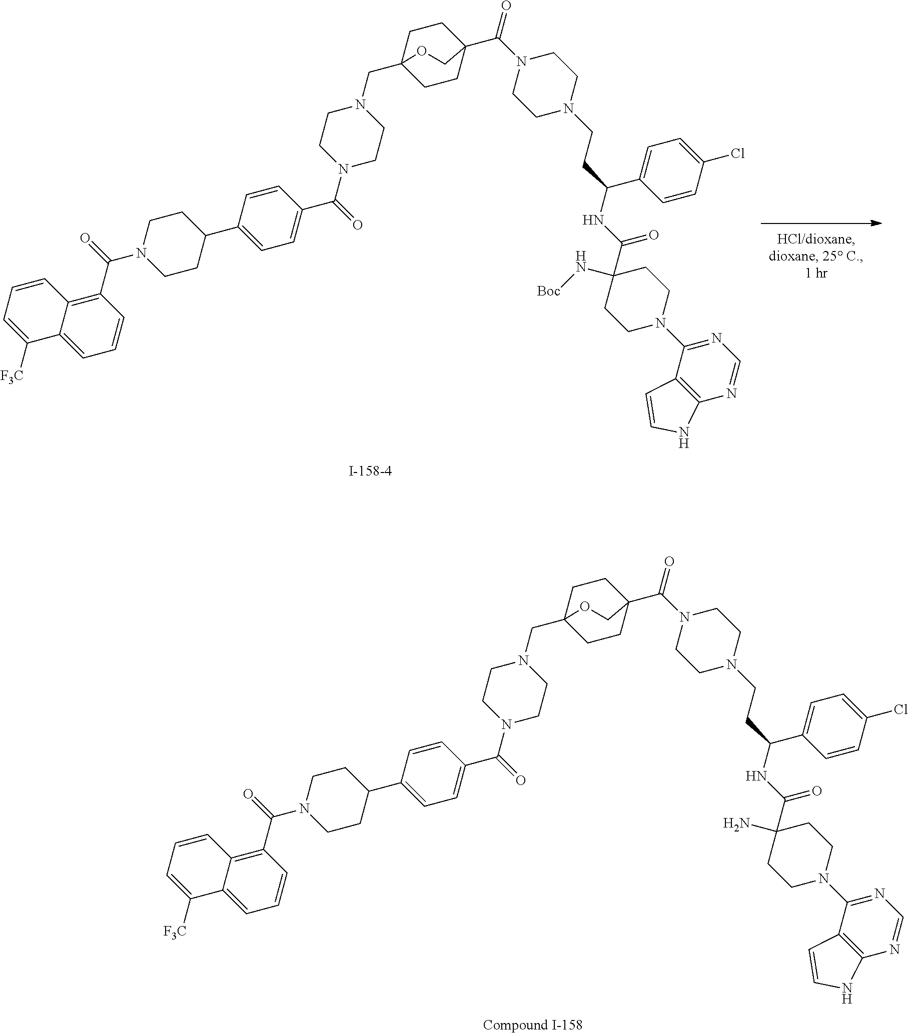 [SMILES: CC(C)(C)OC(=O)NC1(C(=O)N[C@@H](CCN2CCN(C(=O)C34CCC(CN5CCN(C(=O)c6ccc(C7CCN(C(=O)c8cccc9c(C(F)(F)F)cccc89)CC7)cc6)CC5)(CC3)OC4)CC2)c2ccc(Cl)cc2)CCN(c2ncnc3[nH]ccc23)CC1 ; NC1(C(=O)N[C@@H](CCN2CCN(C(=O)C34CCC(CN5CCN(C(=O)c6ccc(C7CCN(C(=O)c8cccc9c(C(F)(F)F)cccc89)CC7)cc6)CC5)(CC3)OC4)CC2)c2ccc(Cl)cc2)CCN(c2ncnc3[nH]ccc23)CC1]

### Step 1: Synthesis of I-158-2

To a solution of I-158-1a (100 mg, 233.96 μmol, 1 eq), EDCI (134.55 mg, 701.89 μmol, 3 eq) and HOAt (95.54 mg, 701.89 μmol, 98.19 μL, 3 eq) in DMF (1 mL) was added I-158-1 (94.18 mg, 350.95 μmol, 1.5 eq) and NMM (118.32 mg, 1.17 mmol, 128.61 μL, 5 eq), the mixture was stirred at 25° C. for 2 hr. The residue was diluted with H₂O (20 mL) and was extracted with ethyl acetate (25 mL*2). The combined organic phase was washed with brine (10 mL*1), dried with anhydrous Na₂SO₄, filtered and concentrated in vacuum. The crude product was purified by reverse-phase HPLC (0.1% FA condition) to give I-158-2 (150 mg, 221.32 μmol, 94.60% yield) as white solid. LCMS (Method E): R^{t}=0.502 min, [M+H]⁺=678.4.

### Step 2: Synthesis of I-158-3

To a solution of methyl I-158-2 (150 mg, 221.32 μmol, 1 eq) in THF (2 mL), MeOH (2 mL) and H₂O (2 mL) was added LiOH·H₂O (46.44 mg, 1.11 mmol, 5 eq), the mixture was stirred at 25° C. for 2 hr. The reaction mixture was adjusted pH to 7˜8 and then extracted with EA, the organic phase was collected and concentrated to give crude product I-158-3 (150 mg, 216.96 μmol, 98.03% yield, 96% purity) as white solid. R^{t}=0.473 min, [M+H]⁺=664.5.

### Step 3: Synthesis of I-158-4

To a solution of I-158-3 (50 mg, 75.33 μmol, 1 eq), EDCI (43.32 mg, 226.00 μmol, 3 eq) and HOAt (30.76 mg, 226.00 μmol, 31.61 μL, 3 eq) in DMF (1 mL) was added NMM (38.10 mg, 376.66 μmol, 41.41 μL, 5 eq) and I-158-3a (67.48 mg, 113.00 μmol, 1.5 eq), the mixture was stirred at 25° C. for 16 hr. The residue was diluted with H₂O (10 mL) and was extracted with ethyl acetate (5 mL*2). The combined organic phase was washed with brine (10 mL*1), dried with anhydrous Na₂SO₄, filtered and concentrated in vacuum to give I-158-4 (100 mg, 60.34 μmol, 80.10% yield, 75% purity) as yellow oil. R^{t}=0.471 min, [M+H]⁺=1242.7.

### Step 4: Synthesis of I-158

To a solution of I-158-4 (270 mg, 217.24 μmol, 1 eq) in dioxane (3 mL) was added HCl/dioxane (3 mL), the mixture was stirred at 25° C. for 1 hr. The reaction mixture was concentrated under reduced pressure to give the crude product. The crude product was purified by Prep-HPLC (column: Phenomenex luna C18 150*25 mm*10 um; mobile phase: [water (HCl)-ACN]; gradient: 13%-43% B over 10 min) to give I-158 (77.15 mg, 65.22 μmol, 30.02% yield, 96.6% purity) as white solid. LCMS (Method G): R^{t}=0.718 min, [M+H]⁺=1142.8. SFC: R^{t}=1.153 min. ¹H NMR (400 MHZ, DMSO-d₆) δ=12.56-12.34 (m, 1H), 12.13-11.04 (m, 1H), 9.58 (m, 1H), 9.31-9.11 (m, 2H), 8.32 (s, 1H), 8.16 (m, 1H), 8.06 (d, J=7.2 Hz, 1H), 7.84-7.59 (m, 3H), 7.52-7.47 (m, 2H), 7.42-7.36 (m, 7H), 6.88 (d, J=3.2 Hz, 1H), 4.99 (m, 1H), 4.92-4.80 (m, 1H), 4.50-4.28 (m, 2H), 4.28-4.14 (m, 4H), 4.13-3.99 (m, 3H), 3.98-3.82 (m, 7H), 3.27-3.00 (m, 10H), 2.91 (brt, J=10.8 Hz, 2H), 2.67-2.53 (m, 2H), 2.44-2.36 (m, 1H), 2.28 (m, 1H), 2.23-1.95 (m, 8H), 1.93-1.81 (m, 3H), 1.81-1.70 (m, 3H), 1.70-1.27 (m, 2H).

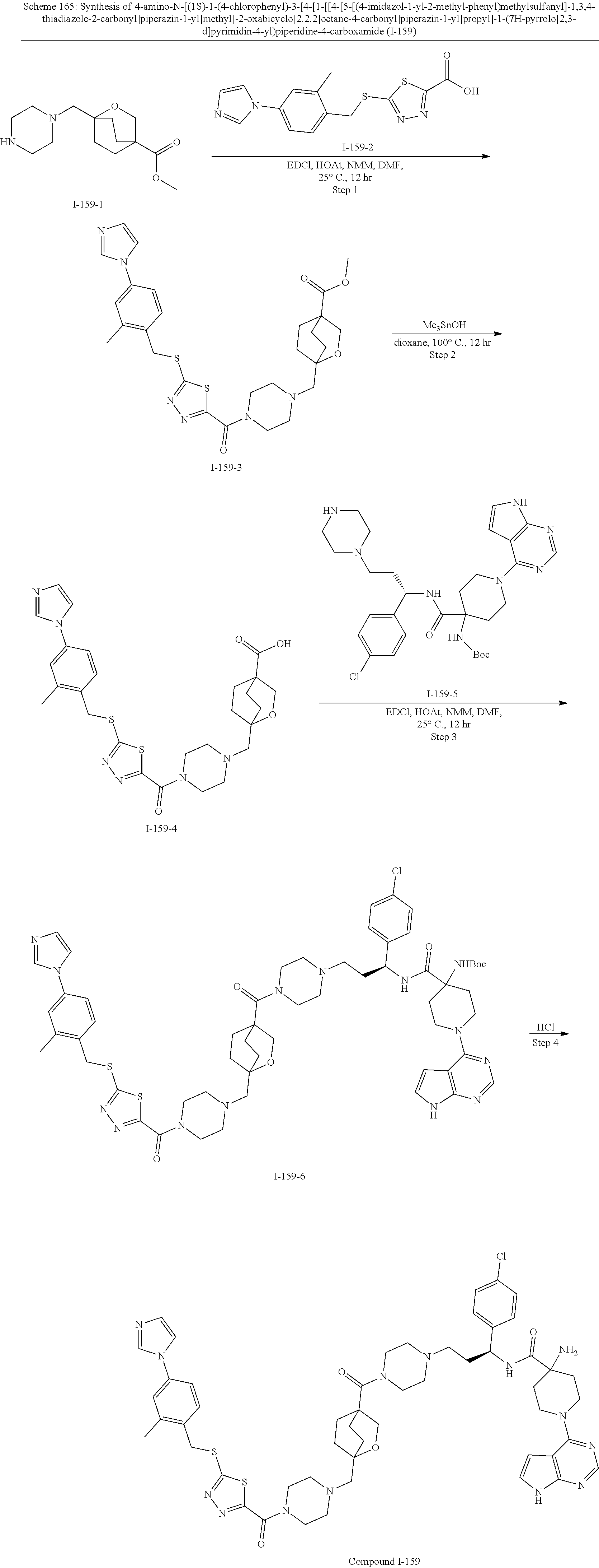

### Step 1: Synthesis of I-159-3

To a solution of I-159-2 (100 mg, 300.84 μmol, 1 eq) in DMF (1 mL) was added EDCI (173.02 mg, 902.53 μmol, 3 eq), NMM (152.15 mg, 1.50 mmol, 165.38 μL, 5 eq), HOAt (122.84 mg, 902.53 μmol, 126.25 μL, 3 eq) and I-159-1 (137.55 mg, 451.26 μmol, 1.5 eq, HCl). The mixture was stirred at 25° C. for 12 hrs. The reaction mixture was quenched by addition H₂O 10 mL and extracted with EA 30 mL (10 mL*3). The combined organic layers were washed with aqueous NaCl 20 mL (10 mL*2), dried over Na₂SO₄, filtered and concentrated under reduced pressure to give I-159-3 (200 mg, crude) as a yellow oil. LCMS (Method G): R^{t}=0.674 min, [M+H]⁺=583.2.

### Step 2: Synthesis of I-159-4

To a solution of I-159-3 (150 mg, 257.41 μmol, 1 eq) in dioxane (1.5 mL) was added Me₃SnOH (372.36 mg, 2.06 mmol, 8 eq). The mixture was stirred at 100° C. for 12 hrs. The reaction mixture was filtered and concentrated under reduced pressure to give I-159-4 (140 mg, crude) as a yellow oil. LCMS (Method G): R^{t}=0.379 min, [M+H]⁺=567.2.

### Step 3: Synthesis of I-159-6

To a solution of I-159-4 (35 mg, 61.54 μmol, 1 eq) in DMF (1 mL) was added EDCI (35.39 mg, 184.63 μmol, 3 eq), HOAt (25.13 mg, 184.63 μmol, 25.83 μL, 3 eq), NMM (31.13 mg, 307.71 μmol, 33.83 μL, 5 eq) and I-159-5 (55.13 mg, 92.31 μmol, 1.5 eq). The mixture was stirred at 25° C. for 12 hrs. The reaction mixture was quenched by addition H₂O 10 mL and extracted with EA 30 mL (10 mL*3). The combined organic layers were washed with aqueous NaCl 20 mL (10 mL*2), dried over Na₂SO₄, filtered and concentrated under reduced pressure to give I-159-6 (60 mg, crude) as a white solid. LCMS (Method G): R^{t}=0.712 min, [M+H]⁺=1147.7.

### Step 4: Synthesis of I-159

To a solution of I-159-6 (60 mg, 52.27 μmol, 1 eq) in dioxane (0.5 mL) was added HCl/dioxane (4 M, 1.20 mL, 91.83 eq). The mixture was stirred at 25° C. for 0.5 hr. The reaction mixture was concentrated under reduced pressure to give a residue. The residue was purified by prep-HPLC (column: Phenomenex luna C18 150*25 mm*10 um; mobile phase: [water (HCl)-ACN]; gradient: 1%-25% B over 10 min) to give I-159 (7.82 mg, 7.46 μmol, 14.28% yield, 100% purity) as an off-white solid. LCMS (Method E): R^{t}=0.369 min, [M+H]⁺=1047.7. ¹H NMR (400 MHZ, METHANOL-d4) δ=9.48 (d, J=1.2 Hz, 1H), 8.41 (s, 1H), 8.17-8.07 (m, 1H), 7.79 (d, J=1.2 Hz, 1H), 7.75 (d, J=8.4 Hz, 1H), 7.67 (s, 1H), 7.57 (br d, J=8.4 Hz, 1H), 7.49-7.42 (m, 3H), 7.37 (d, J=8.4 Hz, 2H), 7.03 (d, J=3.6 Hz, 1H), 5.07 (br dd, J=4.4 Hz, 2H), 4.78 (s, 4H), 4.63 (br d, J=13.2 Hz, 4H), 4.08 (s, 3H), 4.04-3.92 (m, 3H), 3.90-3.50 (m, 6H), 3.49-3.36 (m, 3H), 3.29-3.05 (m, 4H), 2.94-2.81 (m, 1H), 2.81-2.69 (m, 1H), 2.64-2.46 (m, 4H), 2.40-2.22 (m, 4H), 2.18-1.99 (m, 5H), 1.96-1.79 (m, 2H) SFC: Method details: “Column: Chiralcel OJ-3 50*4.6 mm I.D., 3 um; Mobile phase: Phase A for CO₂, and Phase B for EtOH (0.05% DEA); Gradient elution: 60% EtOH (0.05% DEA) in CO₂, Flow rate: 3 mL/min; Detector: PDA; Colum Temp: 35 C; Back Pressure: 100 Bar”; (R^{t}=0.873).

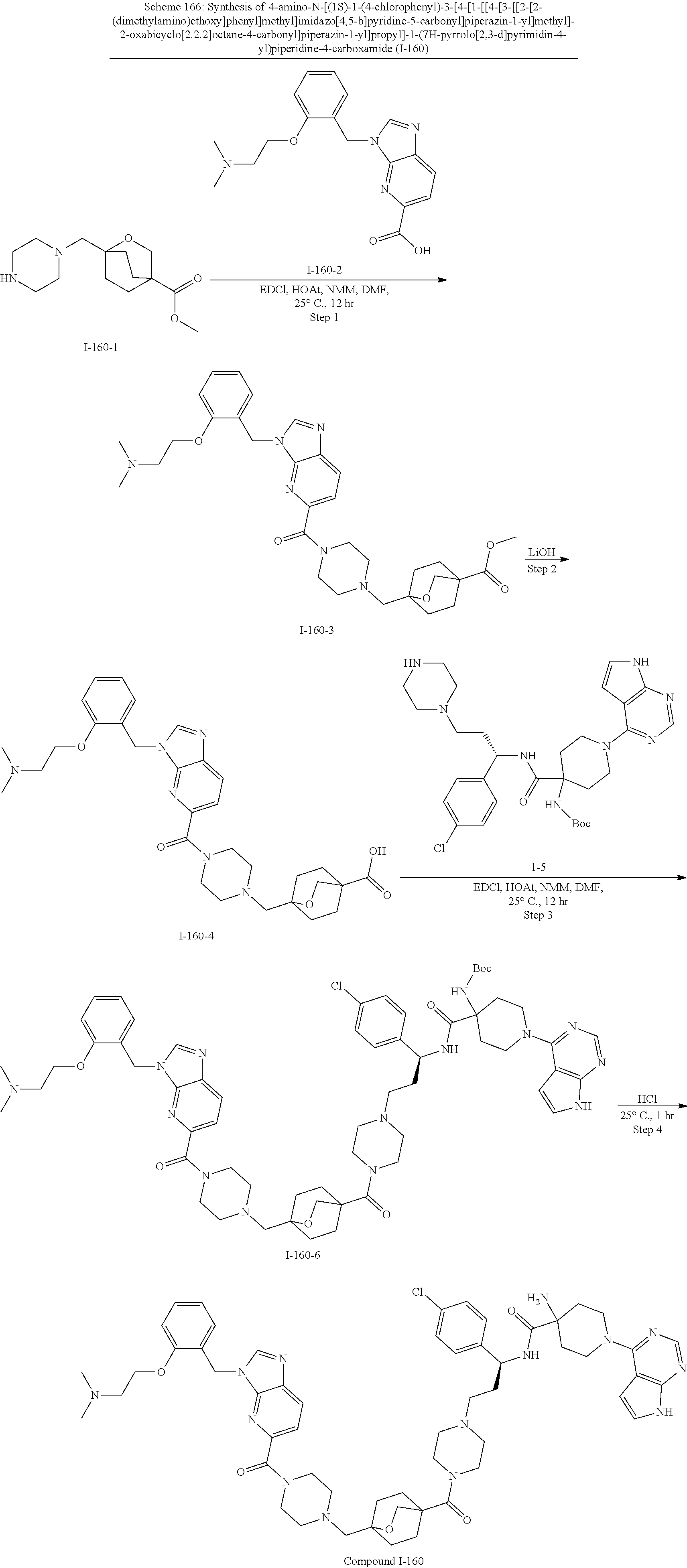

### Step 1: Synthesis of I-160-3

To a solution of I-160-2 (200 mg, 587.59 μmol, 1 eq) in DMF (0.5 mL) was added EDCI (337.92 mg, 1.76 mmol, 3 eq), HOAt (239.93 mg, 1.76 mmol, 246.59 μL, 3 eq), NMM (297.16 mg, 2.94 mmol, 323.00 μL, 5 eq) and I-160-1 (268.65 mg, 881.38 μmol, 1.5 eq, HCl). The mixture was stirred at 25° C. for 12 hrs. The reaction mixture was quenched by addition H₂O 20 mL and extracted with CHCl3:isopropanol=3:1 30 mL (10 mL*3). The combined organic layers were washed with aqueous NaCl 40 mL (20 mL*2), dried over Na₂SO₄, filtered and concentrated under reduced pressure to give I-160-3 (370 mg, crude) as a yellow oil. LCMS (Method G): R^{t}=0.630 min, [M+H]⁺=591.4.

### Step 2: Synthesis of I-160-4

To a solution of I-160-3 (370 mg, 626.36 μmol, 1 eq) in H2O (1 mL), THF (1 mL) and MeOH (1 mL) was added LiOH·H₂O (78.85 mg, 1.88 mmol, 3 eq). The mixture was stirred at 25° C. for 1 hr. The reaction mixture was concentrated under reduced pressure to remove MeOH and THE, and the liquid was lyophilized to give the product. The residue was purified by prep-HPLC (HCl condition) and the organic phase was concentrated under reduced pressure to remove acetonitrile, and the liquid was lyophilized to give I-160-4 (200 mg, 329.47 μmol, 52.60% yield, 95% purity) as a white solid. LCMS (Method E): R^{t}=0.338 min, [M+H]⁺=577.4.

### Step 3: Synthesis of I-160-6

To a solution of I-160-4 (80 mg, 138.72 μmol, 1 eq) in DMF (0.5 mL) was added HOAt (56.65 mg, 416.17 μmol, 58.22 μL, 3 eq), EDCI (79.78 mg, 416.17 μmol, 3 eq), NMM (70.16 mg, 693.62 μmol, 76.26 μL, 5 eq) and I-160-5 (124.26 mg, 208.09 μmol, 1.5 eq). The mixture was stirred at 25° C. for 2 hrs. The reaction mixture was quenched by addition H₂O 20 mL and extracted with CHCl3:isopropanol=3:1 30 mL (10 mL*3). The combined organic layers were washed with aqueous NaCl 40 mL (20 mL*2), dried over Na₂SO₄, filtered and concentrated under reduced pressure to give a residue. The residue was purified by prep-HPLC (basic condition) and the organic phase was concentrated under reduced pressure to remove acetonitrile, and the liquid was lyophilized to give I-160-6 (75 mg, crude) as a white solid. LCMS (Method G): R^{t}=0.689 min, [M+H]⁺=1155.8.

### Step 4: Synthesis of I-160

To a solution of I-160-6 (50 mg, 43.26 μmol, 1 eq) in dioxane (1 mL) was added HCl/dioxane (4 M, 1.25 mL, 115.58 eq). The mixture was stirred at 25° C. for 1 hr. The reaction mixture was concentrated under reduced pressure to give a residue. The residue was purified by prep-HPLC (column: Phenomenex luna C18 150*25 mm*10 um; mobile phase: [water (HCl)-ACN]; gradient: 1%-25% B over 10 min). I-160 (20.39 mg, 19.31 μmol, 44.65% yield, 100% purity) as a white solid. LCMS (Method E): R^{t}=0.358 min, [M+H]⁺=1055.8. ¹H NMR (400 MHz, METHANOL-d4) δ=9.59 (br d, J=4.4 Hz, 1H), 8.46 (d, J=8.4 Hz, 1H), 8.41 (s, 1H), 8.01 (d, J=8.4 Hz, 1H), 7.51-7.41 (m, 4H), 7.41-7.33 (m, 3H), 7.22 (d, J=8.4 Hz, 1H), 7.09 (t, J=7.2 Hz, 1H), 7.04 (d, J=3.6 Hz, 1H), 5.94 (br s, 2H), 5.07 (br dd, J=4.8 Hz, 1H), 4.64 (br d, J=11.2 Hz, 5H), 4.55-4.46 (m, 2H), 4.25-3.94 (m, 5H), 3.86 (br s, 3H), 3.72-3.52 (m, 5H), 3.38 (br s, 2H), 3.33 (td, J=1.6 Hz, 6H), 3.17 (br d, J=10.4 Hz, 2H), 3.05-2.95 (m, 6H), 2.94-2.82 (m, 1H), 2.82-2.70 (m, 1H), 2.63-2.47 (m, 1H), 2.42-2.22 (m, 4H), 2.21-1.97 (m, 5H), 1.96-1.81 (m, 2H). SFC Data: Method details: “Column: Chiralcel OJ-3 50*4.6 mm I.D., 3 um: Mobile phase: Phase A for CO2, and Phase B for EtOH (0.05% DEA): Gradient elution: EtOH (0.05% DEA) in CO2 from 5% to 40%, Flow rate: 3 mL/min: Detector: PDA; Colum Temp: 35 C; Back Pressure: 100 Bar”; (R^{t}=2.233).

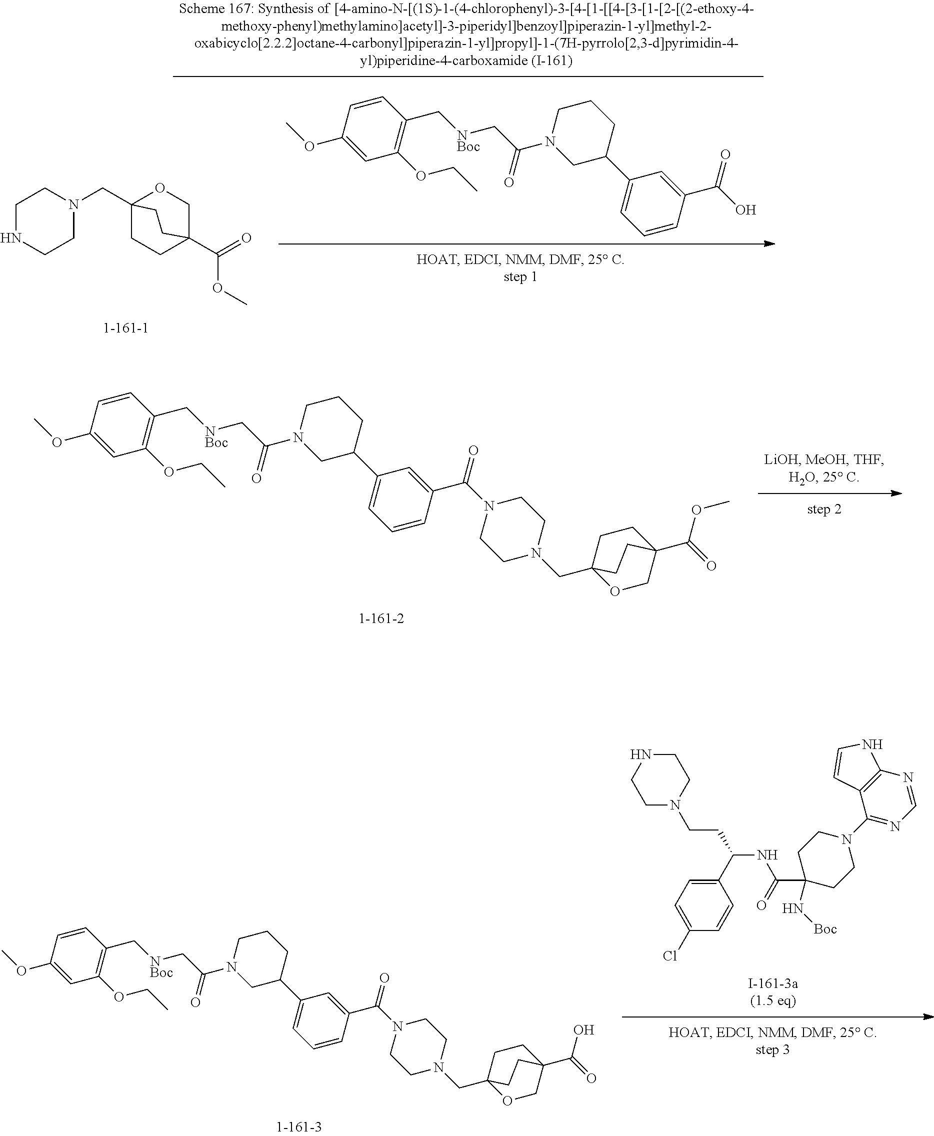

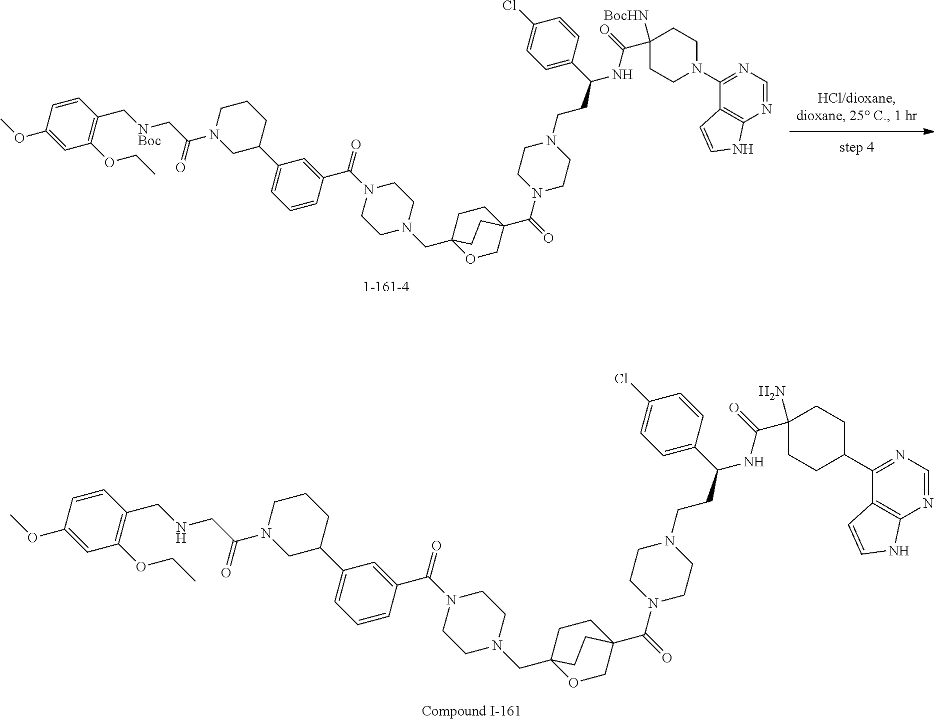 [SMILES: CCOc1cc(OC)ccc1CN(CC(=O)N1CCCC(c2cccc(C(=O)N3CCN(CC45CCC(C(=O)N6CCN(CC[C@H](NC(=O)C7(NC(=O)OC(C)(C)C)CCN(c8ncnc9[nH]ccc89)CC7)c7ccc(Cl)cc7)CC6)(CC4)CO5)CC3)c2)C1)C(=O)OC(C)(C)C ; CCOc1cc(OC)ccc1CNCC(=O)N1CCCC(c2cccc(C(=O)N3CCN(CC45CCC(C(=O)N6CCN(CC[C@H](NC(=O)C7(N)CCC(c8ncnc9[nH]ccc89)CC7)c7ccc(Cl)cc7)CC6)(CC4)CO5)CC3)c2)C1]

### Step 1: Synthesis of I-161-2

To a solution of I-161-1 (100 mg, 189.89 μmol, 1 eq) in DMF (1 mL) was added EDCI (109 mg, 569.67 μmol, 3 eq), HOAt (78 mg, 569.67 μmol, 79.69 μL, 3 eq) and NMM (96 mg, 949.45 μmol, 104.39 μL, 5 eq). The mixture was stirred at 25° C. for 12 hr. The reaction mixture was concentrated under reduced pressure to give the crude product. The residue was purified by prep-HPLC (0.1% NH₃·H₂O) and the organic phase was concentrated under reduced pressure to remove acetonitrile, and the liquid was lyophilized to give I-161-2 (110 mg, 141.58 μmol, 74.56% yield) as a white solid. LCMS (Method E): R^{t}=0.522 min, [M+H]⁺=777.5.

### Step 2: Synthesis of I-161-3

To a solution of I-161-2 (110 mg, 142 μmol, 1 eq) in MeOH (0.5 mL), THF (0.5 mL) and H₂O (0.5 mL) was added LiOH·H₂O (18 mg, 424.73 μmol, 3 eq). The mixture was stirred at 25° C. for 2 hr. The reaction mixture was concentrated under reduced pressure to give I-161-3 (135 mg, crude) as a white solid. LCMS (Method E): R^{t}=0.502 min, [M+H]⁺=763.5.

### Step 3: Synthesis of I-161-4

To a solution of I-161-3 (110 mg, 144.18 μmol, 1 eq) in DMF (1 mL) was added HOAt (59 mg, 432.54 μmol, 60.51 μL, 3 eq), EDCI (83 mg, 432.54 μmol, 3 eq), NMM (73 mg, 720.91 μmol, 79.26 μL, 5 eq) and tert-butyl N-[4-[(1S)-1-(4-chlorophenyl)-3-piperazin-1-ylpropyl]carbamoyl]-1-(7H-pyrrolo[2,3-d]pyrimidin-4-yl)-4-piperidyl]carbamate (129 mg, 216.27 μmol, 1.5 eq). The mixture was stirred at 25° C. for 2 hr. The reaction mixture was diluted into water (5 mL) and extracted with ethyl acetate 9 mL (3 mL×3). The combined organic layers were washed with brine 10 mL (5 mL×2), dried over anhydrous sodium sulfate, filtered and concentrated under reduced pressure to give I-161-4 (150 mg, crude) as a yellow solid.

LCMS (Method G): R^{t}=0.791 min, [M+H]⁺=1342.9. ( )

### Step 4: Synthesis of I-161

To a solution of I-161-4 (150 mg, 111.77 μmol, 1 eq) in dioxane (1 mL) was added HCl/dioxane (4 M, 1 mL, 35.79 eq). The mixture was stirred at 25° C. for 1 hr. The mixture was combined with another batch to work up. The reaction mixture was concentrated under reduced pressure to give the crude product. The residue was dissolved in N,N-dimethylformamide (2 mL) and purified by prep-HPLC (column: Waters Xbridge 150*25 mm*5 um; mobile phase: [water (ammonia hydroxide v/v)-ACN]; gradient: 30%-60% B over 10 min) and the organic phase was concentrated under reduced pressure to remove acetonitrile, and the liquid was lyophilized to give I-161 (39.31 mg, 33.39 μmol, 29.88% yield, 97% purity) as a white solid LCMS (Method G): R^{t}=0.726 min, [M+H]⁺=1141.8. SFC: R^{t}=3.316 min. ¹H NMR (400 MHZ, METHANOL-d4) δ=8.12 (s, 1H), 7.48 (s, 2H), 7.35-7.24 (m, 6H), 7.20-7.08 (m, 2H), 6.61 (d, J=3.6 Hz, 1H), 6.56-6.43 (m, 2H), 5.01-4.93 (m, 1H), 4.56-4.43 (m, 3H), 4.13-4.00 (m, 2H), 3.92 (s, 2H), 3.87-3.53 (m, 15H), 3.45-3.35 (m, 2H), 3.18-2.99 (m, 1H), 2.80-2.57 (m, 4H), 2.56-2.28 (m, 10H), 2.25-2.09 (m, 4H), 2.07-1.75 (m, 9H), 1.70-1.51 (m, 5H), 1.48-1.35 (m, 3H).

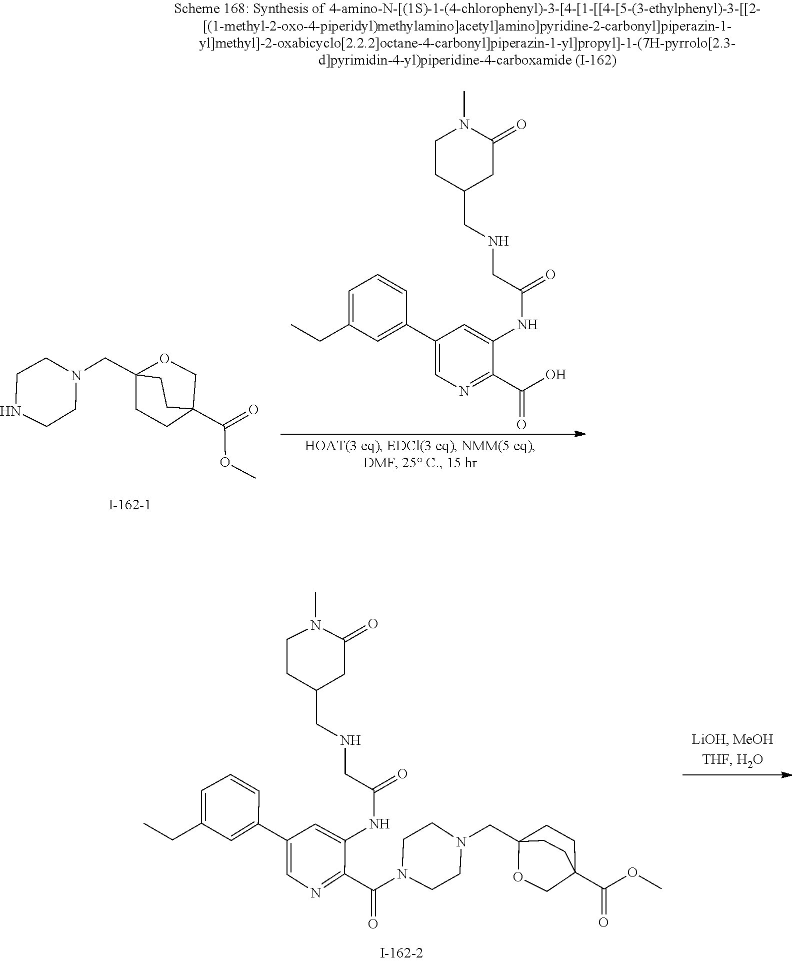 [SMILES: COC(=O)C12CCC(CN3CCNCC3)(CC1)OC2 ; CCc1cccc(-c2cnc(C(=O)O)c(NC(=O)CNCC3CCN(C)C(=O)C3)c2)c1 ; CCc1cccc(-c2cnc(C(=O)N3CCN(CC45CCC(C(=O)OC)(CC4)CO5)CC3)c(NC(=O)CNCC3CCN(C)C(=O)C3)c2)c1]

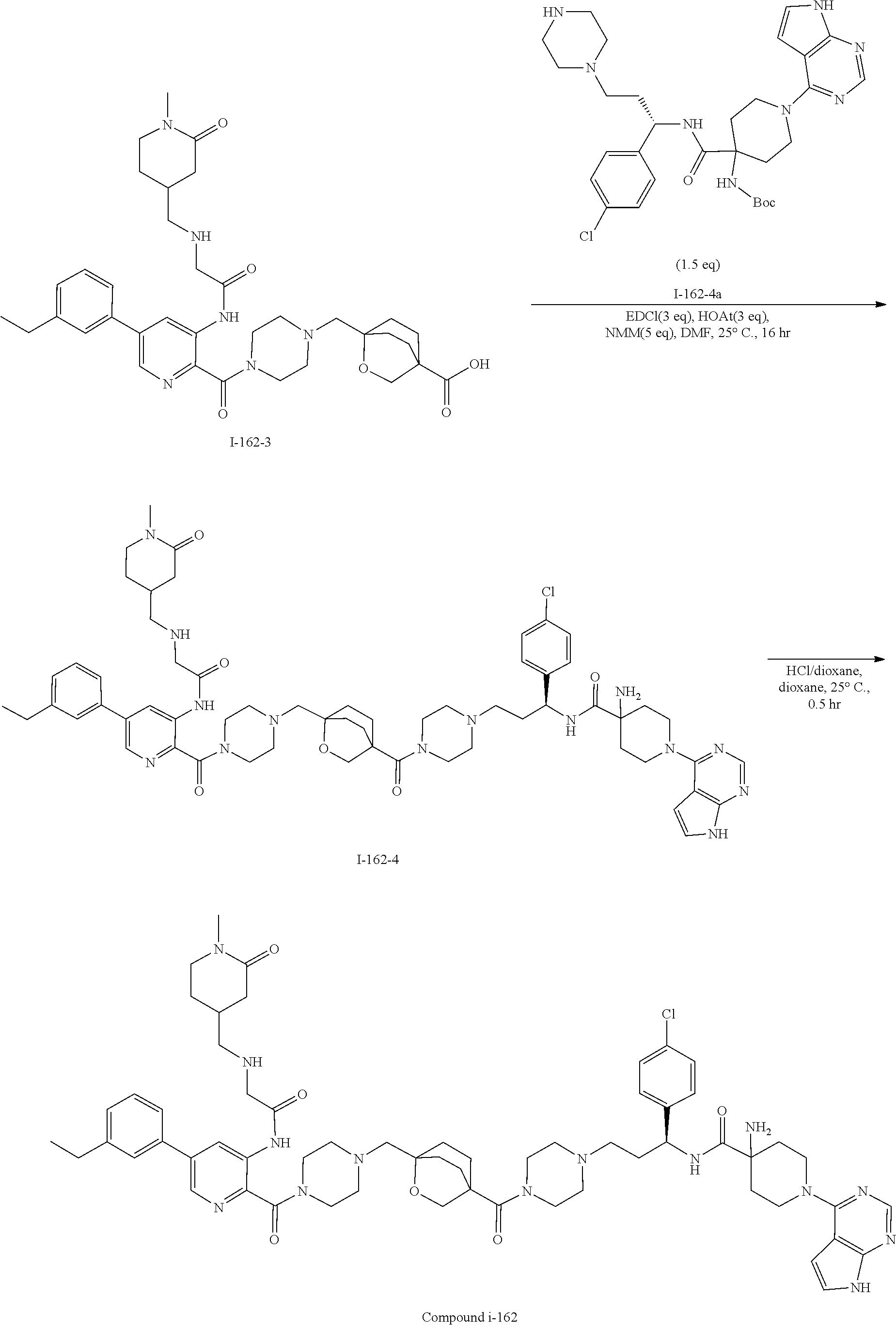 [SMILES: CCc1cccc(-c2cnc(C(=O)N3CCN(CC45CCC(C(=O)O)(CC4)CO5)CC3)c(NC(=O)CNCC3CCN(C)C(=O)C3)c2)c1 ; CC(C)(C)OC(=O)NC1(C(=O)N[C@@H](CCN2CCNCC2)c2ccc(Cl)cc2)CCN(c2ncnc3[nH]ccc23)CC1 ; CCc1cccc(-c2cnc(C(=O)N3CCN(CC45CCC(C(=O)N6CCN(CC[C@H](NC(=O)C7(N)CCN(c8ncnc9[nH]ccc89)CC7)c7ccc(Cl)cc7)CC6)(CC4)CO5)CC3)c(NC(=O)CNCC3CCN(C)C(=O)C3)c2)c1 ; CCc1cccc(-c2cnc(C(=O)N3CCN(CC45CCC(C(=O)N6CCN(CC[C@H](NC(=O)C7(N)CCN(c8ncnc9[nH]ccc89)CC7)c7ccc(Cl)cc7)CC6)(CC4)CO5)CC3)c(NC(=O)CNCC3CCN(C)C(=O)C3)c2)c1]

### Step 1: Synthesis of I-162-2

To a solution of I-162-1a (100 mg, 190.62 μmol, 1 eq) and I-162-1 (76.73 mg, 285.93 μmol, 1.5 eq) in DMF (1 mL) was added EDCI (109.63 mg, 571.86 μmol, 3 eq) HOAt (77.84 mg, 571.86 μmol, 80.00 μL, 3 eq) and NMM (96.40 mg, 953.09 μmol, 104.79 μL, 5 eq). The mixture was stirred at 25° C. for 15 hr. The reaction mixture was diluted with 1 mL DMF and filtered. The filtrate was used for purification. The crude product was purified by reverse-phase HPLC (0.1% FA condition) to give I-162-2 (90 mg, 114.98 μmol, 60.32% yield, 99% purity) as an off-white solid LCMS (Method G): R^{t}=0.737 min, M+H]⁺=775.5. (3).

### Step 2: Synthesis of I-162-3

To a solution of I-162-2 (90 mg, 116.14 μmol, 1 eq) in MeOH (1 mL), THF (1 mL) and H₂O (1 mL) was added LiOH·H₂O (48.74 mg, 1.16 mmol, 10 eq). The mixture was stirred at 25° C. for 1 hr. The reaction mixture was concentrated under reduced pressure and then freeze-dried to give I-162-3 (80 mg, 105.14 μmol, 90.53% yield) as a white solid. LCMS (Method E) R^{t}=0.475 min, [M+H]⁺=761.5.

### Step 3: Synthesis of I-162-5

To a solution of I-162-3 (40 mg, 52.57 μmol, 1 eq), I-162-4a (47.09 mg, 78.85 μmol, 1.5 eq) in DMF (1 mL) was added EDCI (30.23 mg, 157.70 μmol, 3 eq) NMM (26.59 mg, 262.84 μmol, 28.90 μL, 5 eq) and HOAt (21.47 mg, 157.70 μmol, 22.06 μL, 3 eq). The mixture was stirred at 25° C. for 15 hr. The reaction mixture was washed with water (10 mL) and extracted with EA (4 mL*2). The combined organic phase was washed with brine (10 mL*1), dried with anhydrous Na₂SO₄, filtered and concentrated in vacuum to give I-162-5 (70 mg, 52.24 μmol, 99.37% yield) as a yellow oil. LCMS (Method E): R^{t}=0.475 min, [M+H]⁺=1340.4.

### Step 4: Synthesis of I-162

To a solution of I-162-5 (70 mg, 52.24 μmol, 1 eq) in dioxane (0.5 mL) was added HCl/dioxane (4 M, 1 mL, 76.57 eq). The mixture was stirred at 25° C. for 1 hr. The reaction mixture was concentrated under reduced pressure to give a brown foam. The brown foam was purified by prep-HPLC (column: Phenomenex Luna C18 150*25 mm*10 um; mobile phase: [water (HCl)-ACN]; gradient: 5%-35% B over 10 min) to obtain a solution and then freeze-dried to give I-162 (16.76 mg, 14.41 μmol, 27.59% yield, 98% purity) as a brown solid. LCMS (Method G): R^{t}=0.672 min, [M+H]⁺=1139.8. ¹H NMR (400 MHZ, METHANOL-d4) δ=8.72 (d, J=2.0 Hz, 1H), 8.34 (s, 1H), 8.23 (d, J=2.0 Hz, 1H), 7.56-7.48 (m, 2H), 7.47-7.40 (m, 3H), 7.35 (m, 4H), 6.93 (d, J=3.6 Hz, 1H), 5.09-5.01 (m, 1H), 4.65-4.58 (m, 2H), 4.14 (s, 2H), 4.05 (s, 2H), 3.98-3.78 (m, 5H), 3.53-3.41 (m, 4H), 3.37 (br s, 3H), 3.13 (s, 4H), 2.95 (s, 3H), 2.75 (d, J=7.6 Hz, 3H), 2.69-2.39 (m, 4H), 2.35-2.09 (m, 8H), 2.03 (br d, J=12.0 Hz, 3H), 1.86 (br s, 2H), 1.76-1.61 (m, 1H), 1.29 (t, J=7.6 Hz, 3H).

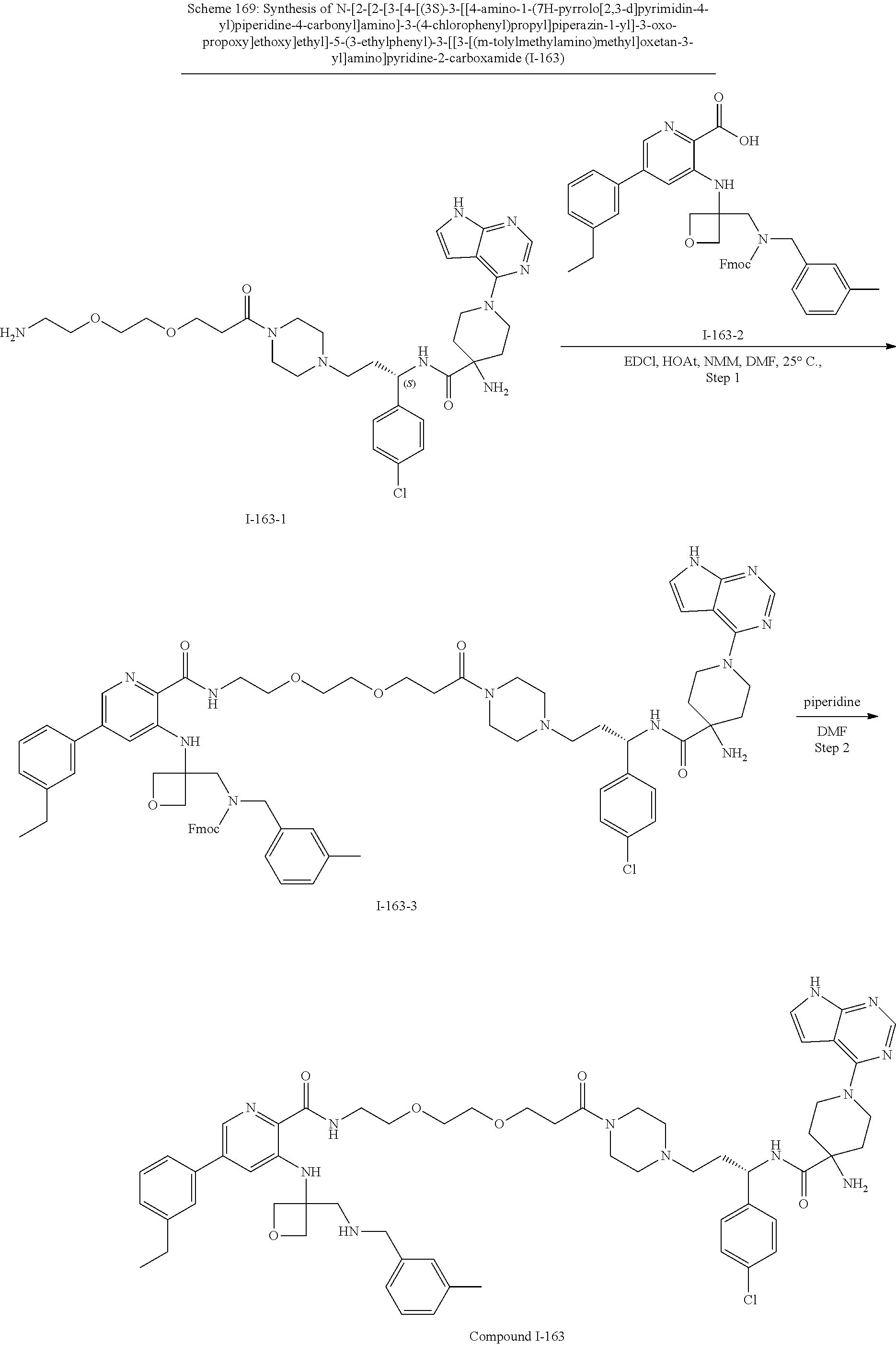

### Step 1: Synthesis of I-163-3

To a solution of I-163-2 (28.31 mg, 43.31 μmol, 1 eq) in DMF (0.3 mL) was added HOAt (11.79 mg, 86.62 μmol, 12.12 μL, 2 eq) and EDCI (41.51 mg, 216.55 μmol, 5 eq). NMM (43.81 mg, 433.10 μmol. 47.62 μL, 10 eq), I-163-1 (30 mg, 43.31 μmol, 1 eq, HCl). The mixture was stirred at 25° C. for 20 min. The reaction mixture was washed with water (5 mL) and extracted with EA (1 mL*3), the combined organic phase was dried by Na₂SO₄, concentrated under reduced pressure to give a residue. The crude product was used into the next step without further purification. I-163-3 (40 mg, 30.96 μmol, 71.49% yield) was obtained as a yellow solid. LCMS: R^{t}=0.531 min, [M+H]⁺=1291.7.

### Step 2: Synthesis of I-163

To a solution of I-163-3 (30 mg, 23.22 μmol, 1 eq) in DMF (0.1 mL) was added piperidine (3.95 mg, 46.44 μmol, 4.59 μL, 2 eq). The mixture was stirred at 25° C. for 1 h. The reaction mixture was purified by prep-HPLC (column: Waters Xbridge 150*25 mm*5 um; mobile phase: [water (NH₃H₂O)-ACN]; gradient: 48%-78% B over 10 min), the eluent was concentrated to remove ACN and lyophilized to get product. I-163 (3 mg, 2.77 μmol, 11.92% yield, 98.733% purity) was obtained as a white solid. LCMS: R^{t}=0.341 min, [M+H]⁺=1069.4. SFC: R^{t}=1.904 min. ¹H NMR (400 MHZ, METHANOL-d4) δ=8.12-8.08 (m, 2H), 7.38-7.33 (m, 1H), 7.31-7.23 (m, 7H), 7.10 (d, J=3.6 Hz, 1H), 7.04-6.99 (m, 1H), 6.98-6.93 (m, 2H), 6.89 (d, J=7.2 Hz, 1H), 6.64 (s, 1H), 6.59 (d, J=3.6 Hz, 1H), 4.93 (s, 1H), 4.74-4.68 (m, 4H), 4.58 (s, 2H), 4.51-4.43 (m, 2H), 3.76 (t, J=6.0 Hz, 2H), 3.71 (s, 2H), 3.69-3.65 (m, 4H), 3.65-3.61 (m, 3H), 3.59 (t, J=4.8 Hz, 5H), 3.53 (s, 2H), 3.16 (s, 2H), 2.75-2.67 (m, 2H), 2.61 (t, J=6.4 Hz, 2H), 2.42-2.23 (m, 6H), 2.13 (s, 3H), 2.05-1.95 (m, 1H), 1.94-1.86 (m, 1H), 1.61-1.49 (m, 2H), 1.26 (t, J=7.6 Hz, 3H).

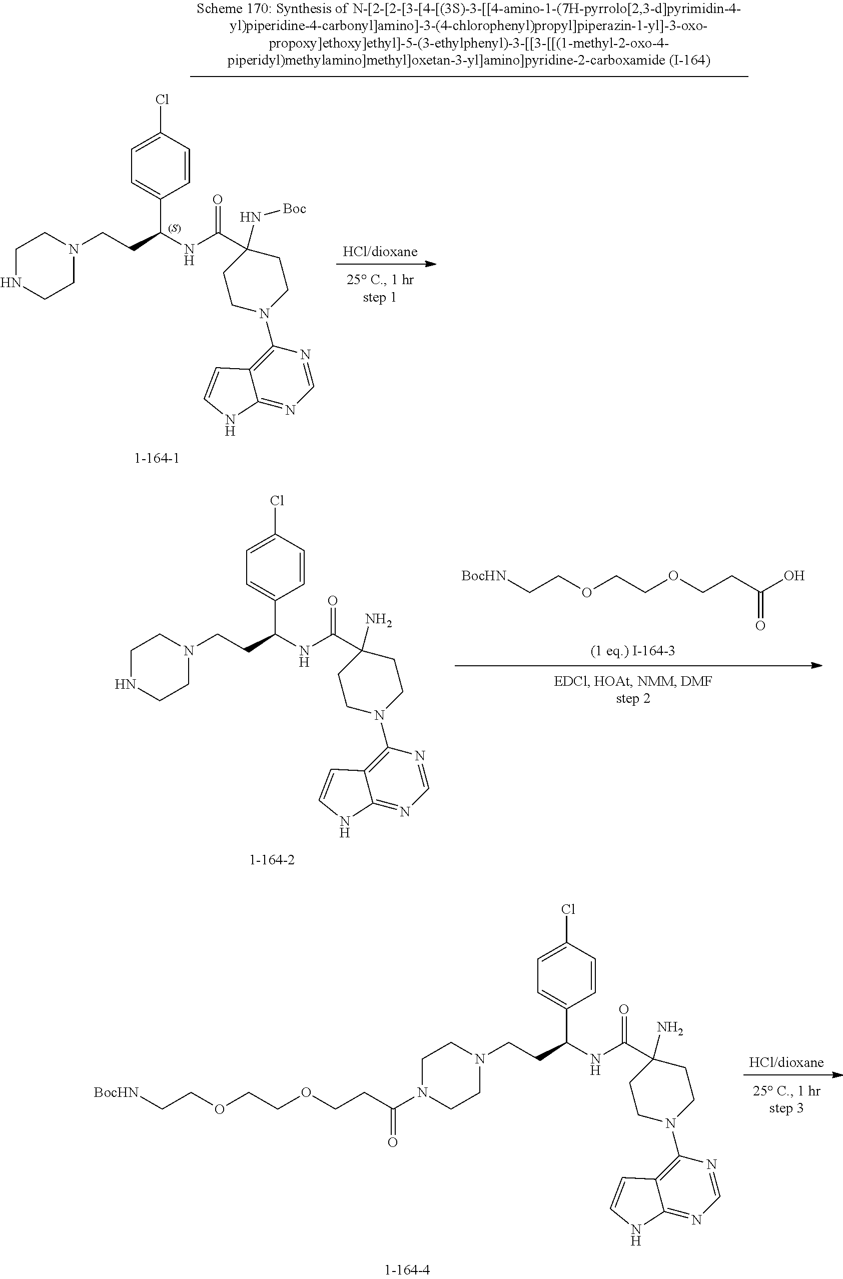

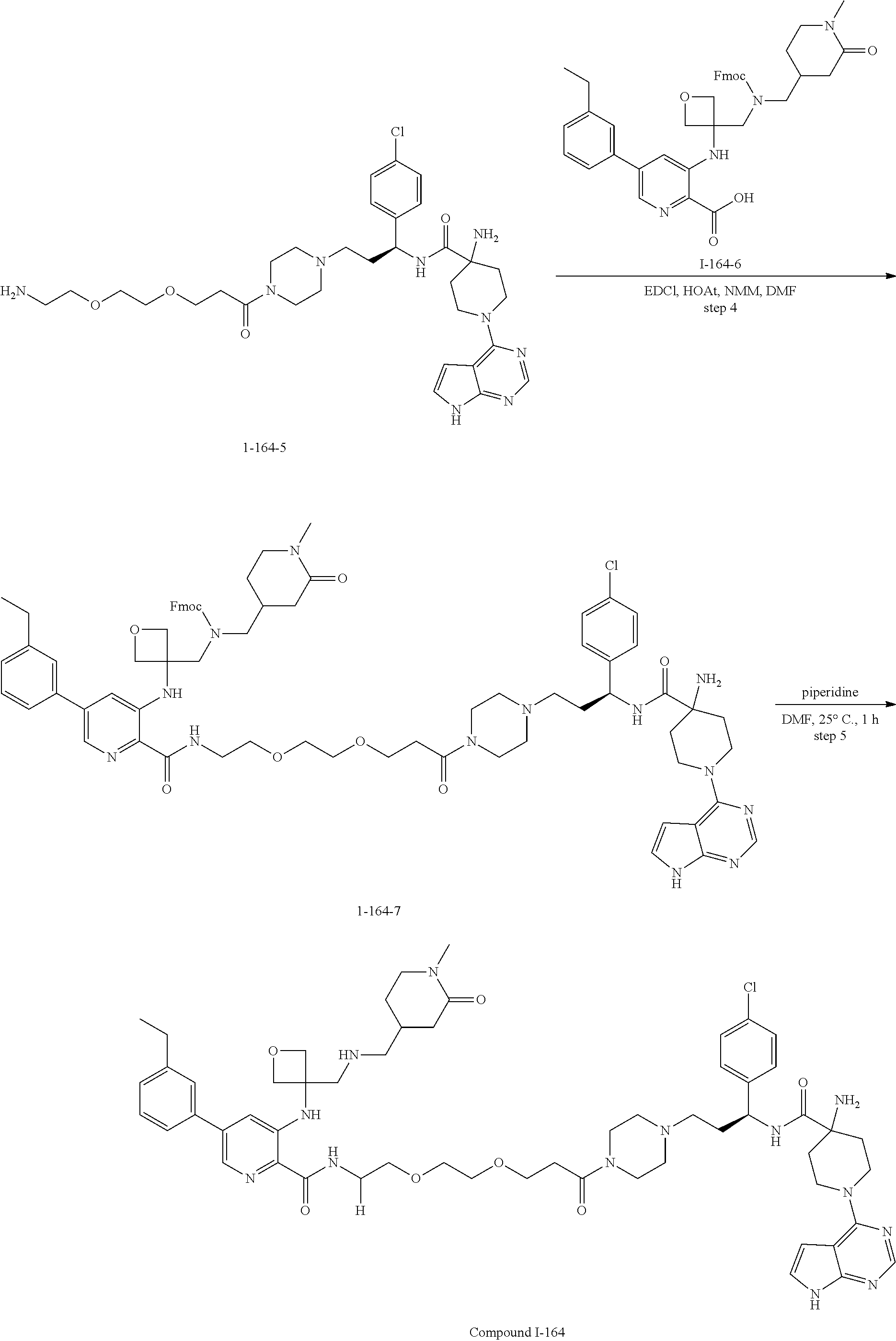

### Step 1: Synthesis of I-164-2

To a solution of I-164-1 (500 mg, 837.31 μmol, 1 eq) in HCl/dioxane (2 M, 5 mL), the mixture was stirred at 25° C. for 1 hr. Concentrated under reduced pressure to give a residue. The crude product was used into the next step without further purification. I-164-2 (570 mg, crude, HCl) was obtained as a yellow solid. LCMS: R^{t}=0.553 min, [M+H]⁺=497.3. ¹H NMR (400 MHZ, DMSO-d₆) δ=12.95 (s, 1H), 10.07-9.94 (m, 1H), 9.76 (d, J=7.6 Hz, 1H), 9.22 (s, 2H), 8.39 (s, 1H), 7.49 (s, 1H), 7.47-7.42 (m, 2H), 7.41-7.37 (m, 2H), 7.03 (d, J=2.0 Hz, 1H), 4.95 (t, J=10.8 Hz, 1H), 4.33-4.18 (m, 4H), 3.75-3.61 (m, 2H), 3.46-3.31 (m, 5H), 3.27-3.18 (m, 1H), 2.62 (d, J=8.4 Hz, 2H), 2.42-2.30 (m, 1H), 2.20 (m, 2H), 2.02 (d, J=14.4 Hz, 1H), 1.58 (s, 2H).

### Step 2: Synthesis of I-164-4

To a solution of I-164-3 (296.29 mg, 1.07 mmol, 1 eq) in DMF (5 mL) was added EDCI (614.45 mg, 3.21 mmol, 3 eq) and HOAt (436.27 mg, 3.21 mmol, 448.38 μL, 3 eq), NMM (540.34 mg, 5.34 mmol, 587.33 μL, 5 eq), I-164-2 (570 mg, 1.07 mmol, 1 eq, HCl). The mixture was stirred at 25° C. for 20 min.

The reaction mixture was filtered to give the filtrate. The residue was purified by reverse-phase (0.1% of FA) and the eluent was lyophilized to give product. I-164-4 (300 mg, 331.31 μmol, 31.01% yield, 83.526% purity) was obtained as a yellow solid. LCMS (Method D): R^{t}=0.255 min, [M+H]⁺=756.3.

### Step 3: Synthesis of I-164-5

To a solution of I-164-4 (150 mg, 198.33 μmol, 1 eq) in HCl/dioxane (2 M, 1.5 mL), the mixture was stirred at 25° C. for 1 hr. The mixture was concentrated under reduced pressure to give a residue. The crude product was used into the next step without further purification. I-164-5 (150 mg, crude, HCl) was obtained as a yellow solid. LCMS (Method D): R^{t}=0.218 min, [M+H]⁺=656.3.

### Step 4: Synthesis of I-164-7

To a solution of I-164-6 (29.22 mg, 43.31 μmol, 1 eq) in DMF (0.3 mL) was added EDCI (41.51 mg, 216.55 μmol, 5 eq) and HOAt (11.79 mg, 86.62 μmol, 12.12 μL, 2 eq), NMM (43.81 mg, 433.10 μmol, 47.62 μL, 10 eq), I-164-5 (30 mg, 43.31 μmol, 1 eq, HCl). The mixture was stirred at 25° C. for 1 h. The reaction mixture was filtered to give the filtrate. The residue was purified by reverse-phase (0.1% of FA) and the eluent was lyophilized to give product. I-164-7 (30 mg, 10.73 μmol, 24.77% yield, 46.949% purity) was obtained as colorless oil. LCMS (Method D): R^{t}=0.421 min, [M+H]⁺=1312.6.

### Step 5: Synthesis of I-164

To a solution of I-164-7 (25 mg, 19.04 μmol, 1 eq) in DMF (0.2 mL) was added piperidine (3.24 mg, 38.08 μmol, 3.76 μL, 2 eq). The mixture was stirred at 25° C. for 1 h. The reaction mixture was filtered to give the filtrate. The crude was purified by prep-HPLC (column: Waters Xbridge 150*25 mm*5 um; mobile phase: [water (NH₃H₂O)-ACN]; gradient: 34%-64% B over 12 min) the eluent was concentrated to remove ACN and lyophilized to get products. I-164 (5 mg, 4.54 μmol, 23.84% yield, 99.029% purity) was obtained as a colorless gum. LCMS: R^{t}=0.627 min, [M+H]⁺=1090.5. SFC: R^{t}=4.895 min, 5.178 min. ¹H NMR (400 MHZ, METHANOL-d4) δ=8.17-8.06 (m, 2H), 7.42 (s, 1H), 7.41-7.37 (m, 2H), 7.32-7.26 (m, 5H), 7.10 (d, J=3.6 Hz, 1H), 6.95 (d, J=1.6 Hz, 1H), 6.58 (d, J=3.6 Hz, 1H), 4.93 (s, 1H), 4.78 (s, 2H), 4.76-4.73 (m, 2H), 4.62-4.54 (m, 2H), 4.52-4.44 (m, 2H), 3.76 (t, J=6.4 Hz, 2H), 3.70-3.51 (m, 15H), 3.27-3.22 (m, 2H), 3.20 (d, J=3.2 Hz, 2H), 2.87 (s, 3H), 2.74-2.68 (m, 2H), 2.61 (t, J=6.4 Hz, 2H), 2.56-2.49 (m, 2H), 2.46-2.39 (m, 2H), 2.38-2.34 (m, 2H), 2.33 (d, J=4.8 Hz, 1H), 2.29-2.24 (m, 2H), 2.20-2.10 (m, 2H), 2.06-1.95 (m, 2H), 1.95-1.89 (m, 3H), 1.60-1.51 (m, 2H), 1.41 (s, 1H), 1.30-1.24 (m, 4H).

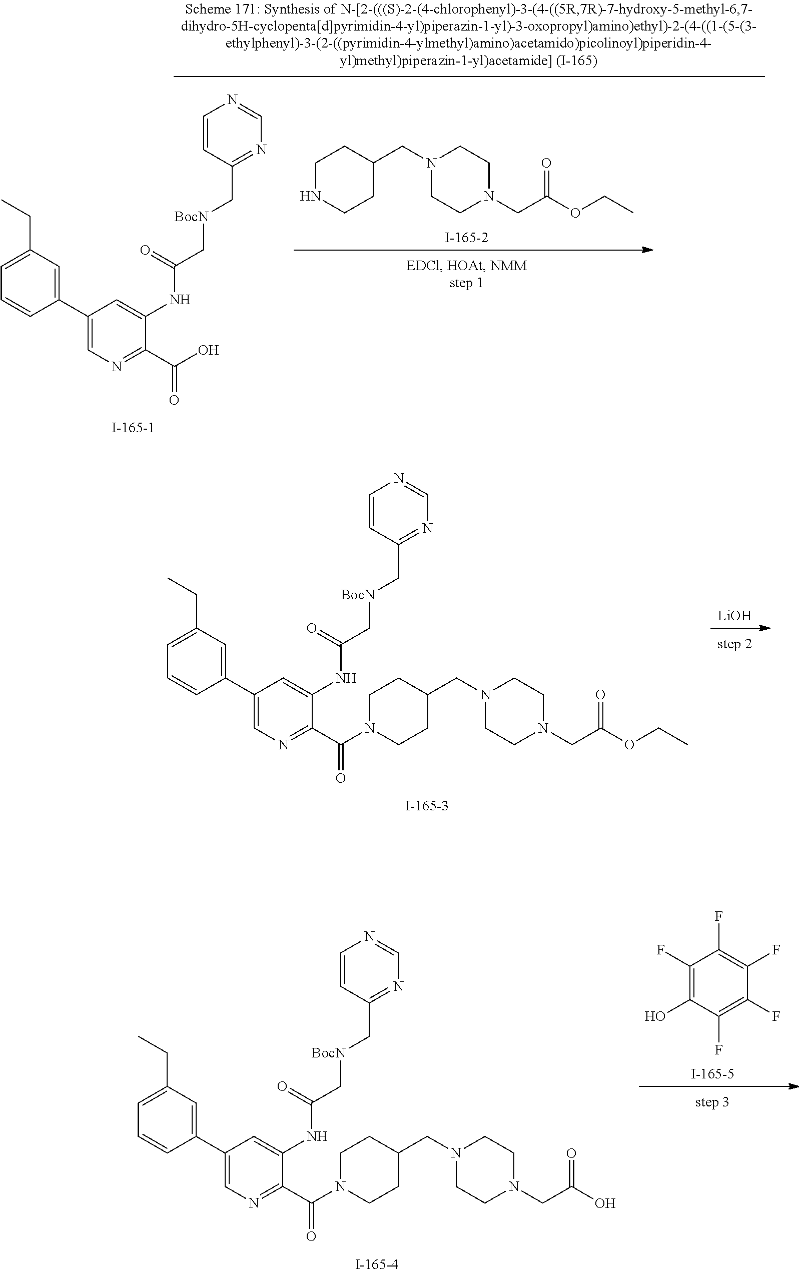

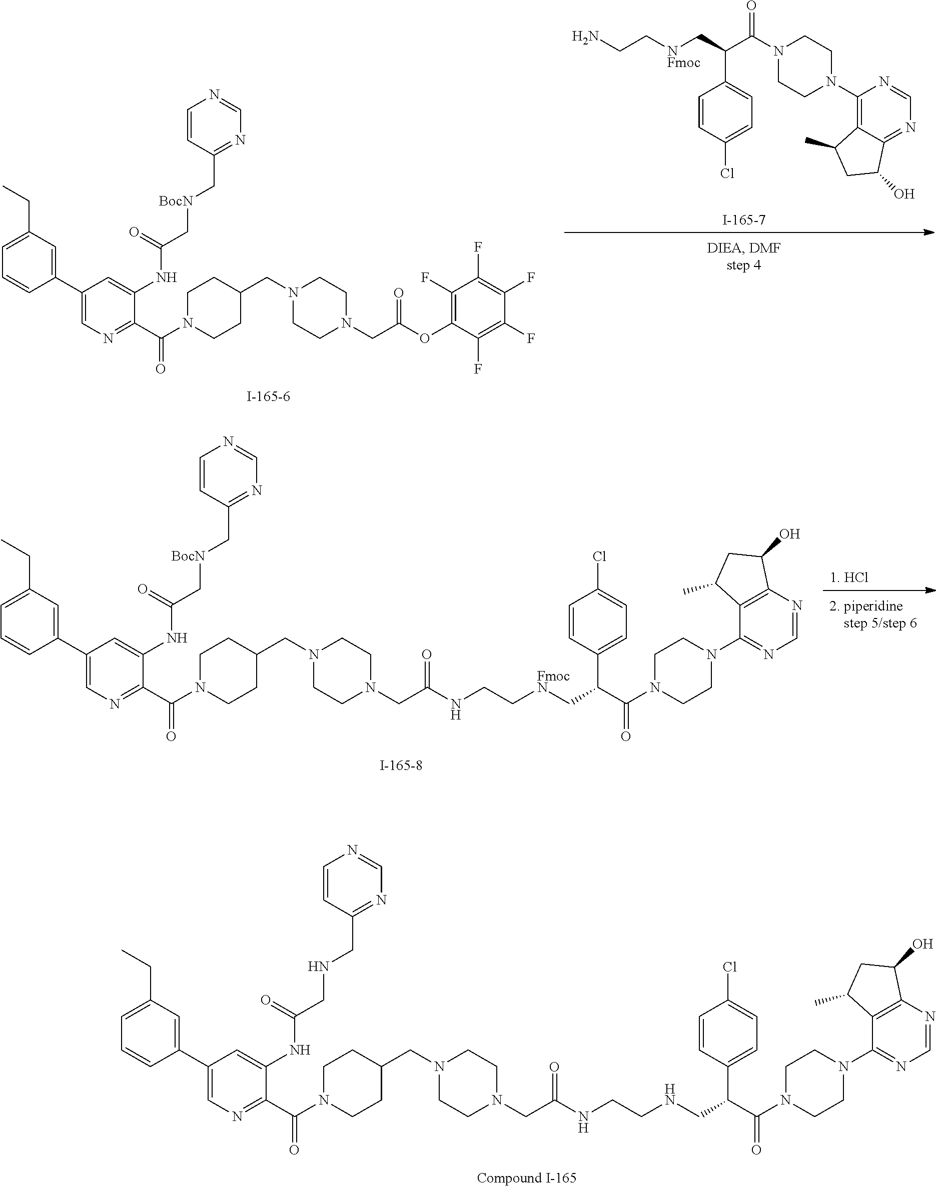

### Step 1: Synthesis of I-165-3

To a solution of I-165-1 (500 mg, 1.02 mmol, 1 eq), I-165-2 (311.11 mg, 1.02 mmol, 1 eq, HCl) in DMF (5 mL) was added EDCI (585.00 mg, 3.05 mmol, 3 eq) and HOAt (138.45 mg, 1.02 mmol, 142.30 μL, 1 eq), NMM (514.44 mg, 5.09 mmol, 559.18 μL, 5 eq). The mixture was stirred at 25° C. for 12 hr. The reaction mixture was diluted with H₂O 10 mL and extracted with EA 60 mL (20 mL*3). The combined organic layers were washed with NaCl (aq) 10 mL (5 mL*2), dried over Na₂SO₄, filtered and concentrated under reduced pressure to give a residue. The residue was purified by prep-HPLC (0.1% FA) the eluent was concentrated to remove ACN and lyophilized to get I-165-3 (500 mg, 673.03 μmol, 66.16% yield) was obtained as a white solid. LCMS (Method D): R^{t}=0.427 min, (M+H)=743.9.

### Step 2: Synthesis of I-165-4

To a solution of I-165-3 (200 mg, 269.21 μmol, 1 eq) in H₂O (1 mL), THF (1 mL), MeOH (1 mL) was added LiOH·H₂O (33.89 mg, 807.64 μmol, 3 eq). The mixture was stirred at 25° C. for 0.5 hr. The reaction mixture was adjusted pH=6 by HCl (2M), and then extracted with EA 10 mL (5 mL*2). The combined organic layers were washed with NaCl (aq) 10 mL (5 mL*2), dried over Na₂SO₄, filtered and concentrated under reduced pressure to give I-165-4 (150 mg, 209.83 μmol, 77.94% yield) was obtained as a colorless oil. LCMS (Method D): R^{t}=0.392 min, (M+H)=715.4.

### Step 3: Synthesis of I-165-6

To a solution of I-165-4 (120 mg, 167.87 μmol, 1 eq), I-165-5 (46.35 mg, 251.80 μmol, 1.5 eq) in DMF (1.5 mL) was added N,N′-diisopropylmethanediimine (31.78 mg, 251.80 μmol, 38.99 μL, 1.5 eq). The mixture was stirred at 25° C. for 0.5 hr. The reaction mixture was extracted with DCM 10 ml (5 mL*2). The combined organic layers were washed with NaCl (aq) 2 mL, dried over Na₂SO₄, filtered and concentrated under reduced pressure to give I-165-6 (0.15 g, crude) was obtained as a yellow solid. LCMS (Method D): R^{t}=0.396 min, (M+H)=881.3.

### Step 4: Synthesis of I-165-8

To a solution of I-165-6 (0.15 g, 170.28 μmol, 1 eq), I-165-7 (97.77 mg, 136.22 μmol, 0.8 eq, HCl) in DMF (1.5 mL) was added DIEA (33.01 mg, 255.42 μmol, 44.49 μL, 1.5 eq). The mixture was stirred at 25° C. for 0.5 hr. The reaction mixture was filtered and concentrated under reduced pressure to give a residue. The residue was purified by prep-HPLC (0.1% FA) the eluent was concentrated to remove ACN and lyophilized to I-165-8 (0.12 g, 87.08 μmol, 51.14% yield) was obtained as a white solid. LCMS (Method D): R^{t}=0.411 min, (M+H)=1377.5. SFC method: R^{t}=2.030 min.

### Step 5: Synthesis of I-165-9

To a solution of I-165-8 (0.12 g, 87.08 μmol, 1 eq) in HCl/dioxane (1 mL). The mixture was stirred at 25° C. for 0.15 hr. The reaction mixture was concentrated under reduced pressure to give I-165-9 (0.12 g, crude, HCl) was obtained as a white solid. LCMS (Method D): R^{t}=0.354 min, (M+H)=1278.4. SFC method: R^{t}=2.164 min.

### Step 6: Synthesis of I-165

To a solution of I-165-9 (0.12 g, 91.30 μmol, 1 eq, HCl) in DMF (1 mL) was added PIPERIDINE (15.55 mg, 182.59 μmol, 18.03 μL, 2 eq). The mixture was stirred at 25° C. for 0.5 hr. The reaction mixture was filtered and concentrated under reduced pressure to give a residue. The residue was purified by prep-HPLC (column: CD20-Waters Xbidge BEH C18 250*25*10 um; mobile phase: [water (NH4HCO3)-ACN]; gradient: 31%-61% B over 15 min) the eluent was concentrated to remove ACN and lyophilized to get I-165 was obtained as a white solid (22.47 mg, 21.14 μmol, 23.15% yield, 99.314% purity). LCMS: R^{t}=1.256 min, (M/2+H)=528.7. SFC method: R^{t}=1.629 min. ¹H NMR (400 MHZ, METHANOL-d4) δ=9.10 (d, J=1.2 Hz, 1H), 8.81 (d, J=2.0 Hz, 1H), 8.77 (d, J=5.2 Hz, 1H), 8.60 (d, J=2.0 Hz, 1H), 8.41 (s, 1H), 7.78 (d, J=4.8 Hz, 1H), 7.55-7.48 (m, 2H), 7.45-7.41 (m, 1H), 7.40-7.36 (m, 2H), 7.34-7.29 (m, 3H), 4.98-4.95 (m, 1H), 4.60 (d, J=12.8 Hz, 1H), 4.20-4.17 (m, 1H), 4.01 (s, 2H), 3.93-3.85 (m, 1H), 3.80 (d, J=13.6 Hz, 1H), 3.76-3.66 (m, 2H), 3.65-3.58 (m, 2H), 3.56-3.54 (m, 1H), 3.52 (s, 2H), 3.50-3.46 (m, 1H), 3.37-3.33 (m, 2H), 3.30-3.23 (m, 3H), 3.17 (s, 1H), 2.99 (s, 2H), 2.89-2.81 (m, 1H), 2.79-2.77 (m, 1H), 2.77-2.75 (m, 2H), 2.74-2.71 (m, 2H), 2.51 (d, J=10.8 Hz, 8H), 2.23 (d, J=6.8 Hz, 2H), 2.16-2.09 (m, 1H), 2.08-2.01 (m, 1H), 1.91-1.82 (m, 2H), 1.76 (d, J=12.0 Hz, 1H), 1.32-1.26 (m, 4H), 1.18-1.13 (m, 1H), 1.11 (d, J=7.2 Hz, 3H).

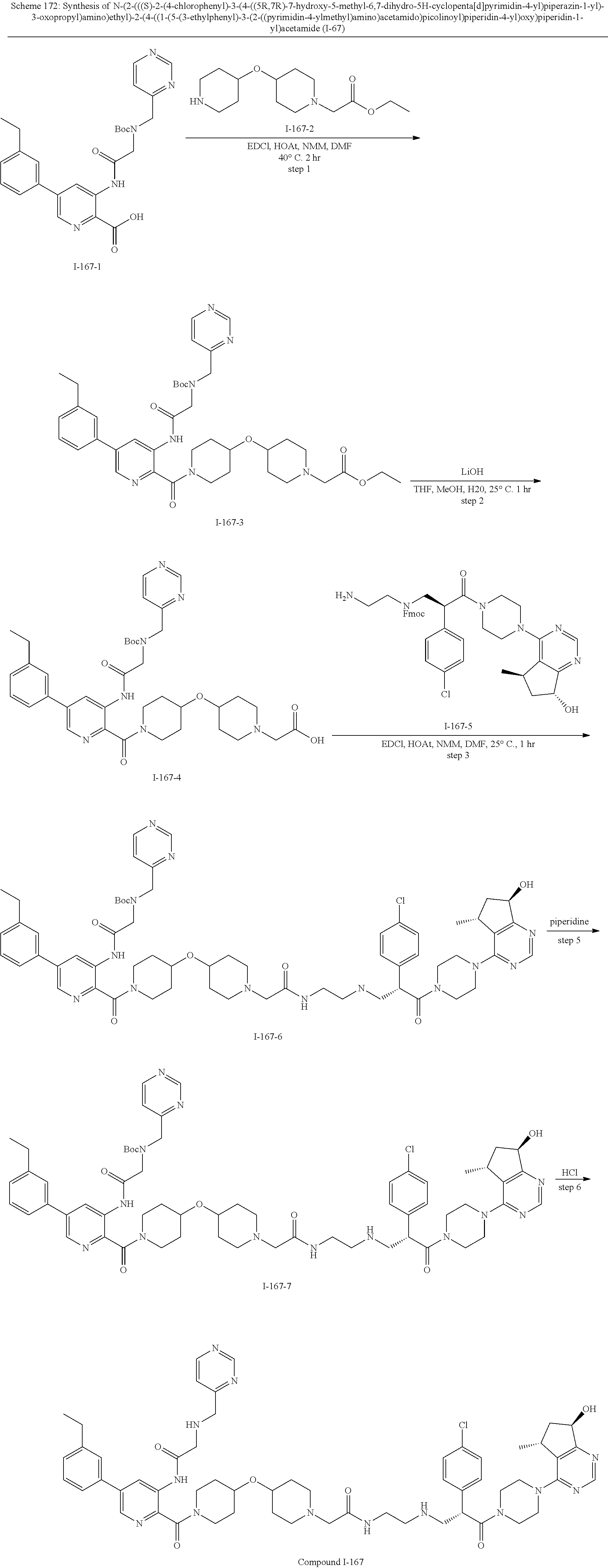

### Step 1: Synthesis of I-167-3

To a solution of I-167-1 (600 mg, 1.22 mmol, 1 eq), I-167-2 (374.53 mg, 1.22 mmol, 1 eq, HCl) in DMF (6 mL) was added EDCI (468.00 mg, 2.44 mmol, 2 eq) and HOAt (166.15 mg, 1.22 mmol, 170.76 μL, 1 eq) and NMM (617.33 mg, 6.10 mmol, 671.01 μL, 5 eq). The mixture was stirred at 25° C. for 1 hr. The reaction mixture was added H₂O (10 mL) and then extracted with DCM (10 mL*3), the combined organic phase was washed with dried by Na₂SO₄, filtered and concentrated. The residue was purified by flash silica gel chromatography (ISCO®; 60 g SepaFlash® Silica Flash Column, Eluent of 0˜100% Ethyl Methanol/Dichloromethane ether gradient @ 80 mL/min) and the eluent was concentrated to give product. I-167-3 (600 mg, 669.45 μmol, 54.84% yield, 83% purity) was obtained as a white solid. LCMS (Method D): R^{t}=0.380 min, [M+H]⁺=744.4.

### Step 2: Synthesis of I-167-3

To a solution of I-167-2 (200 mg, 223.15 μmol, 1 eq) in THF (1 mL) and H₂O (1 mL) and MeOH (1 mL) was added LiOH·H₂O (18.73 mg, 446.30 μmol, 2 eq). The mixture was stirred at 25° C. for 1 hr. The reaction mixture was added H₂O (5 mL) and then extracted with DCM (5 mL*3), the combined organic phase was washed with dried by Na₂SO₄, filtered and concentrated. The crude was used next step. I-167-3 (170 mg, 218.84 μmol, 98.07% yield, 92.150% purity) was obtained as a white solid. LCMS (Method D): R^{t}=0.380 min, [M+H]⁺=716.4.

### Step 3: Synthesis of I-167-5

To a solution of I-167-3 (34.91 mg, 48.77 μmol, 1 eq), I-167-4 (35 mg, 48.77 μmol, 1 eq, HCl) in DMF (0.4 mL) was added EDCI (18.70 mg, 97.54 μmol, 2 eq) and HOAt (6.64 mg, 48.77 μmol, 6.82 μL, 1 eq) and NMM (24.66 mg, 243.84 μmol, 26.81 μL, 5 eq). The mixture was stirred at 25° C. for 1 hr. The reaction mixture was added H₂O (1 mL) and then extracted with DCM (1 mL*3), the combined organic phase was washed with dried by Na₂SO₄, filtered and concentrated. The crude was used next step. I-167-5 (50 mg, 36.26 μmol, 74.35% yield, N/A purity) was obtained as a white solid. LCMS (Method D): R^{t}=0.421 min, [M/2+H]⁺=690.3.

### Step 4: Synthesis of I-167-7

To a solution of I-167-6 (50 mg, 36.26 μmol, 1 eq) in DMF (0.5 mL) was added piperidine (6.17 mg, 72.51 μmol, 7.16 μL, 2 eq). The mixture was stirred at 25° C. for 1 hr. The reaction mixture was diluted with MeOH to purification. The crude product was purified by reverse-phase HPLC (0.1% FA condition), the eluent was concentrated to remove ACN and lyophilized to get product. 30 mg (33% purity) was obtained, then was purified by flash silica gel chromatography (ISCO®; 4 g SepaFlash® Silica Flash Column, Eluent of 0˜100% Ethyl acetate/Petroleum ether gradient @ 12 mL/min) and the eluent was concentrated to give product. I-167-7 (10 mg, 8.64 μmol, 23.84% yield, N/A purity) was obtained as a white solid. LCMS (Method D): R^{t}=0.340 min, [M+H]⁺=1156.5.

### Step 5: Synthesis of I-167

To a solution of I-167-7 (10 mg, 8.64 μmol, 1 eq) in DCM (0.3 mL) was added TFA (153.50 mg, 1.35 mmol, 0.1 mL, 155.73 eq). The mixture was stirred at 25° C. for 1 hr. The reaction mixture was concentrated under reduced pressure to give a residue. The crude product was purified by reverse-phase HPLC (0.1% TFA condition), the eluent was concentrated to remove ACN and lyophilized to get product. I-167 (2.69 mg, 2.26 μmol, 26.18% yield, 98.503% purity, TFA) was obtained as a white solid. LCMS (Method D): R^{t}=0.290 min, [M+H]⁺=1056.4. SFC: R^{t}=1.700 min. ¹H NMR (400 MHZ, METHANOL-d4) δ=9.26 (s, 1H), 8.85 (d, J=5.2 Hz, 1H), 8.69 (s, 1H), 8.56 (d, J=1.6 Hz, 1H), 8.37 (s, 1H), 7.61 (d, J=5.6 Hz, 1H), 7.56-7.41 (m, 5H), 7.40-7.31 (m, 3H), 5.26 (t, J=7.6 Hz, 1H), 4.57 (s, 2H), 4.24-4.09 (m, 3H), 4.08-3.98 (m, 3H), 3.95-3.77 (m, 6H), 3.77-3.52 (m, 9H), 3.51-3.36 (m, 4H), 3.35 (s, 3H), 3.29-3.23 (m, 3H), 2.78-2.72 (m, 2H), 2.32-2.23 (m, 1H), 2.22-2.16 (m, 1H), 2.15-1.95 (m, 5H), 1.80-1.67 (m, 2H), 1.30 (t, J=7.6 Hz, 3H), 1.17 (d, J=7.2 Hz, 3H).

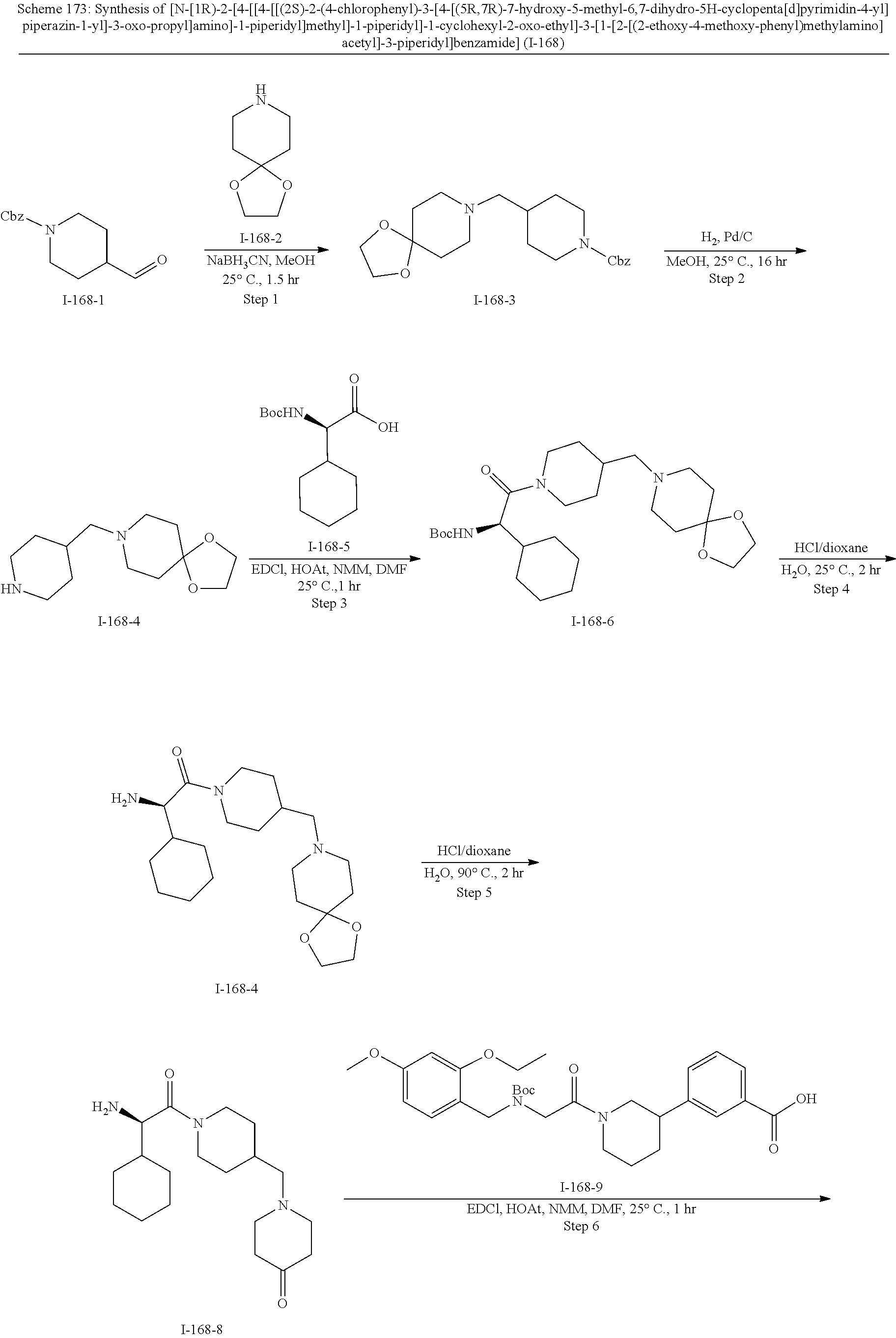

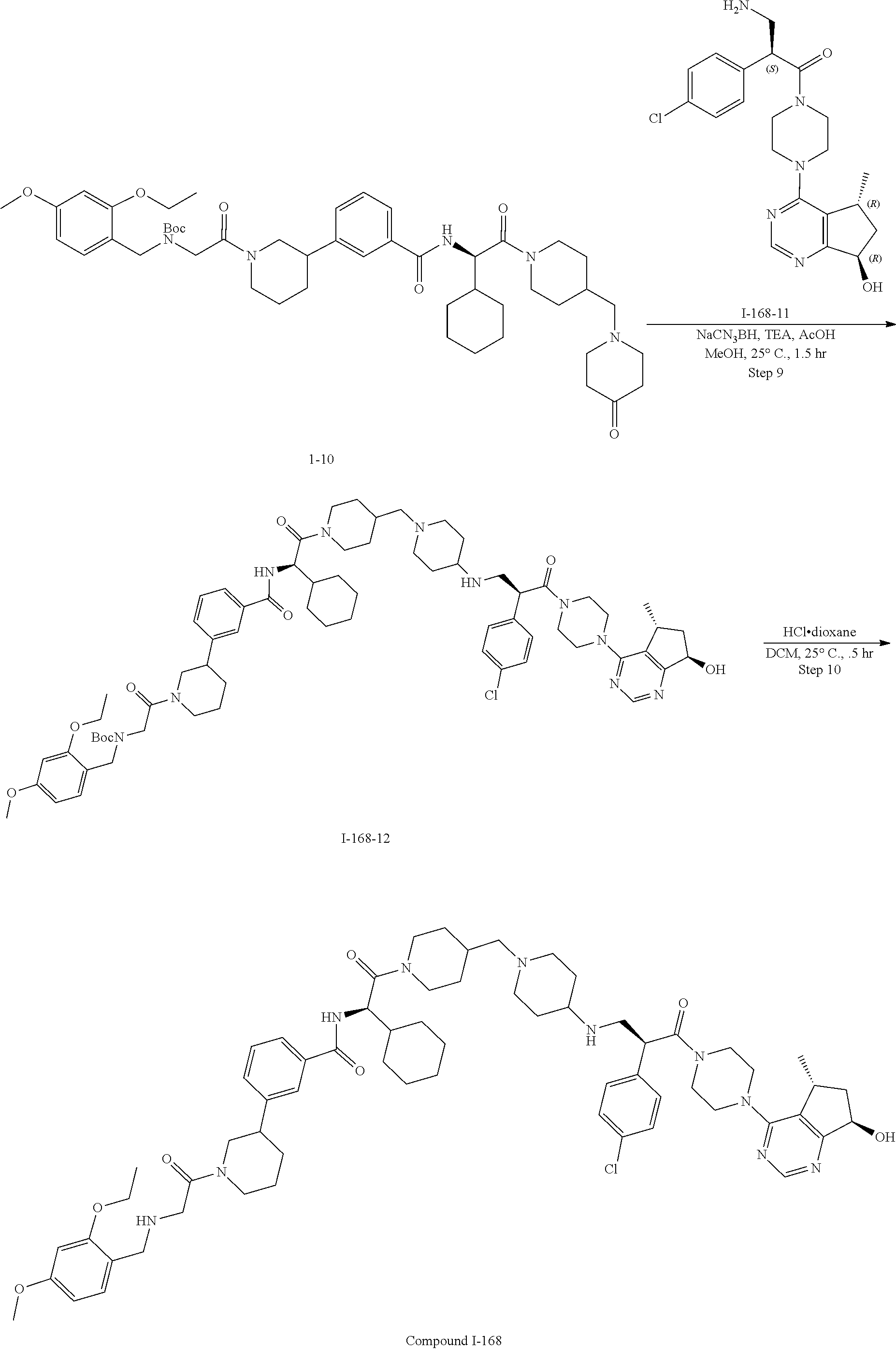

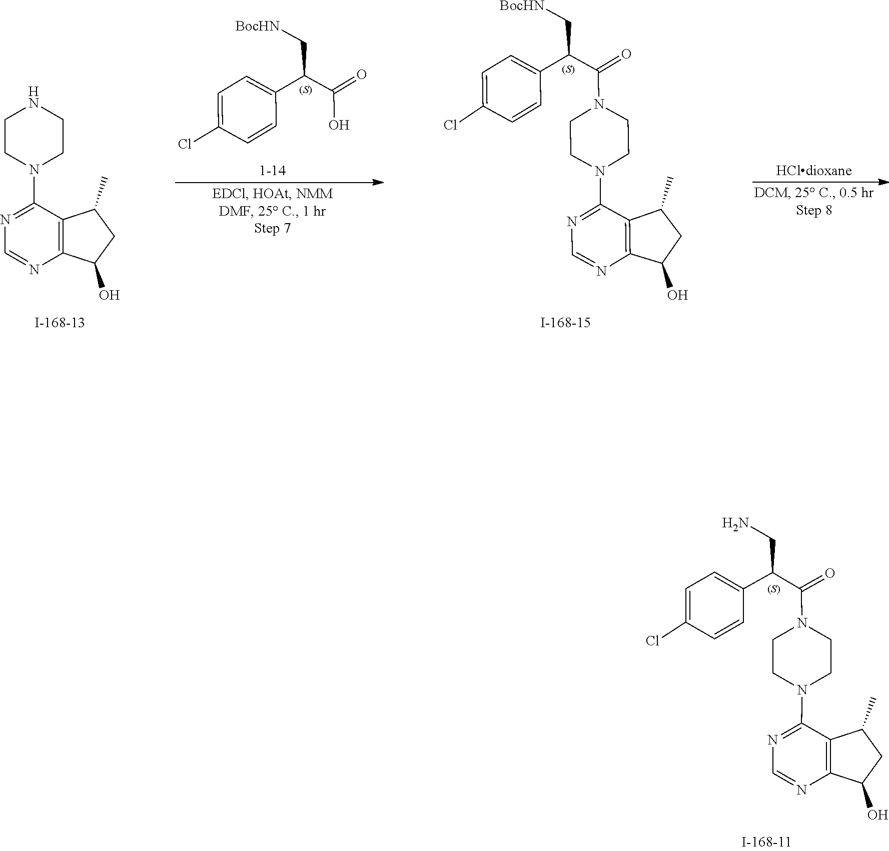

### Step 1: Synthesis of I-168-3.

A mixture of I-168-1 (1 g. 4.04 mmol, 1 eq) and I-168-2 (579.01 mg, 4.04 mmol, 518.36 μL, 1 eq) in MeOH (10 mL) was stirred at 25° C. for 0.5 h, then NaBH₃CN (762.37 mg, 12.13 mmol, 3 eq) was added to the mixture and stirred at 25° C. for 1 h. The reaction mixture was concentrated to give a residue. The residue was purified by reverse-phase HPLC (0.1% FA condition) and lyophilizated to give the product. I-168-3 (740 mg, 1.98 mmol, 48.87% yield, N/A purity) was obtained as a white gum. LCMS (Method D): R^{t}=0.262 min, [M+H]⁺=375.2.

### Step 2: Synthesis of I-168-4.

To a solution of I-168-3 (740 mg, 1.98 mmol, 1 eq) in MeOH (8 mL) was added Pd/C (100 mg, 10% purity) under N₂ atmosphere. The suspension was degassed and purged with H₂ for 3 times. The mixture was stirred under H₂ (1 eq) (15 Psi) at 25° C. for 16 h. The reaction mixture was filtered to remove Pd/C and concentrated to give the crude product. The crude product was used for next step directly. I-168-4 (400 mg, 1.66 mmol, 84.22% yield) was obtained as a white gum. LCMS (Method D): R^{t}=0.297 min, [M+H]⁺=241.2.

### Step 3: Synthesis of I-168-6.

To a mixture of I-168-4 (350 mg, 1.46 mmol, 1 eq) in DMF (3.5 mL) was added I-168-5 (374.73 mg, 1.46 mmol, 1 eq), EDCI (837.50 mg, 4.37 mmol, 3 eq), HOAt (198.21 mg, 1.46 mmol, 203.71 μL, 1 eq) and NMM (736.48 mg, 7.28 mmol, 800.53 μL, 5 eq) at 25° C., the mixture was stirred at 25° C. for 1 h. The reaction mixture was concentrated to give a residue. The residue was purified by reverse-phase HPLC (0.1% FA condition) and lyophilizated to give the product. I-168-6 (490 mg, 1.02 mmol, 70.15% yield, N/A purity) was obtained as a yellow gum. LCMS (Method D): R^{t}=0.309 min, [M+H]⁺=480.3.

### Step 4: Synthesis of I-168-7.

To a mixture of I-168-6 (390 mg, 813.09 μmol, 1 eq) in H₂O (2 mL) was added HCl/dioxane (2 M, 2 mL, 4.92 eq) at 25° C., the mixture was stirred at 25° C. for 1 h. The reaction mixture was concentrated to give the crude product. The crude product was used for next step directly. I-168-7 (340 mg, crude, HCl) was obtained as a white solid. LCMS: R^{t}=0.588 min, [M+H]⁺=380.3.

### Step 5: Synthesis of I-168-8.

To a mixture of I-168-7 (340 mg, 817.32 μmol, 1 eq, HCl) in H₂O (2 mL) was added HCl/dioxane (2 M, 2 mL) at 25° C., the mixture was stirred at 90° C. for 2 h. The reaction mixture was adjusted pH around 8 by aq. NaHCO₃ and extracted with DCM (2 mL×3), the combined organic layers were dried over

Na₂SO₄, filtered and concentrated to give the crude product. The crude product was used for next step directly. I-168-8 (140 mg, crude) was obtained as a yellow solid. LCMS: R^{t}=0.515 min, [M+H]⁺=336.3. ¹H NMR (400 MHZ, METHANOL-d4) δ=4.71-4.44 (m, 2H), 4.35-3.77 (m, 3H), 2.80-2.40 (m, 5H), 2.37-2.17 (m, 2H), 2.02-1.53 (m, 9H), 1.40-0.99 (m, 9H), 0.95-0.83 (m, 1H).

### Step 6: Synthesis of I-168-10.

To a mixture of I-168-8 (130 mg, 387.50 μmol, 1 eq) in DMF (1.5 mL) was added I-168-9 (204.07 mg, 387.50 μmol, 1 eq), EDCI (222.85 mg, 1.16 mmol, 3 eq), HOAt (52.74 mg, 387.50 μmol, 54.21 μL, 1 eq) and NMM (195.97 mg, 1.94 mmol, 213.01 μL, 5 eq) at 25° C., the mixture was stirred at 25° C. for 1 h. The reaction mixture was concentrated to give a residue. The residue was purified by reverse-phase HPLC (0.1% FA condition) and lyophilizated to give the product. I-168-10 (115 mg, 136.24 μmol, 35.16% yield, N/A purity) was obtained as a brown solid. LCMS (Method D): R^{t}=0.415 min, [M+H]⁺=844.5. SFC: R^{t}=0.793 min, 1.741 min.

### Step 7: Synthesis of I-168-15.

To a mixture of I-168-13 (200 mg, 651.00 μmol, 1 eq, 2HCl) in DMF (2 mL) was added I-168-14 (195.14 mg, 651.00 μmol, 1 eq), EDCI (374.39 mg, 1.95 mmol, 3 eq), HOAt (88.61 mg, 651.00 μmol, 91.07 μL, 1 eq) and NMM (329.23 mg, 3.26 mmol, 357.86 μL, 5 eq) at 25° C., the mixture was stirred at 25° C. for 1 h. The reaction mixture was concentrated to give a residue. The residue was purified by column chromatography (SiO₂, EA/MeOH=1/0 to 7/3) and concentrated to give the product. I-168-15 (268 mg, 519.35 μmol, 79.78% yield, N/A purity) was obtained as a yellow solid. LCMS (Method D): R^{t}=0.335 min, [M+H]⁺=516.1. SFC: R^{t}=0.821 min.

### Step 8: Synthesis of I-168-11.

To a mixture of I-168-15 (50 mg, 96.89 μmol, 1 eq) in DCM (0.5 mL) was added HCl/dioxane (2 M, 0.5 mL, 10.32 eq) at 25° C., the mixture was stirred at 25° C. for 0.5 hr. The reaction mixture was concentrated to give the crude product. No purification, the crude product was used for next step directly. I-168-11 (40 mg, crude, HCl) was obtained as a yellow solid. LCMS (Method D): R^{t}=0.211 min, [M+H]⁺=416.2.

### Step 9: Synthesis of I-168-12.

To a mixture of I-168-10 (50 mg, 59.24 μmol, 1 eq) and I-168-11 (32.16 mg, 71.08 μmol, 1.2 eq, HCl) in MeOH (0.5 mL) was added TEA (599.40 μg, 5.92 μmol, 8.24e-1 μL, 0.1 eq) and AcOH (355.72 μg, 5.92 μmol, 3.39e-1 μL, 0.1 eq), the mixture was stirred at 25° C. for 0.5 h, then NaBH₃CN (11.17 mg, 177.71 μmol, 3 eq) was added and stirred at 25° C. for 1 h. The reaction mixture was diluted with H₂O (2 mL) and extracted with DCM (2 mL×3), the combined organic layers were dried over Na₂SO₄, filtered and concentrated to give a residue. The residue was purified by reverse-phase HPLC (0.1% FA condition) and lyophilizated to give the product. I-168-12 (50 mg, 40.19 μmol, 67.85% yield, N/A purity) was obtained as a yellow solid. LCMS (Method D): R^{t}=0.393 min, ([M+H]+/2)=622.8.

### Step 10: Synthesis of I-168

To a mixture of I-168-12 (40 mg, 32.15 μmol, 1 eq) in DCM (0.5 mL) was added HCl/dioxane (2 M, 0.5 mL, 31.10 eq) at 25° C., the mixture was stirred 25° C. for 0.5 h. The reaction mixture was concentrated to give a residue. The residue was purified by prep-HPLC (column: Welch Ultimate C18 150*25 mm*5 um; mobile phase: [water (HCl)-ACN]; gradient: 16%-46% B over 10 min) and lyophilizated to give the product. I-168 (10.94 mg, 9.08 μmol, 28.24% yield, 98.770% purity, FA) was obtained as a white solid. LCMS (Method D): R^{t}=0.317 min, [M+H]⁺=1143.5. SFC: R^{t}=3.354 min, 4.550 min. 1H NMR (400 MHZ, METHANOL-d4) δ=8.54-8.46 (m, 1H), 8.42 (s, 1H), 7.80-7.67 (m, 2H), 7.53-7.40 (m, 4H), 7.36 (s, 2H), 7.31-7.22 (m, 1H), 6.67-6.51 (m, 2H), 5.00-4.93 (m, 2H), 4.61-4.50 (m, 2H), 4.38-4.25 (m, 2H), 4.22-4.07 (m, 4H), 4.07-3.87 (m, 3H), 3.74-3.66 (m, 2H), 3.65-3.43 (m, 6H), 3.27-3.13 (m, 4H), 3.12-2.94 (m, 3H), 2.88-2.68 (m, 3H), 2.57-2.23 (m, 4H), 2.22-2.11 (m, 2H), 2.09-2.01 (m, 4H), 1.86 (d, J=10.8 Hz, 6H), 1.81-1.75 (m, 2H), 1.73-1.58 (m, 5H), 1.51-1.40 (m, 3H), 1.33 (s, 2H), 1.29 (s, 5H), 1.26 (s, 1H), 1.15-1.07 (m, 5H), 0.90 (t, J=6.4 Hz, 1H).

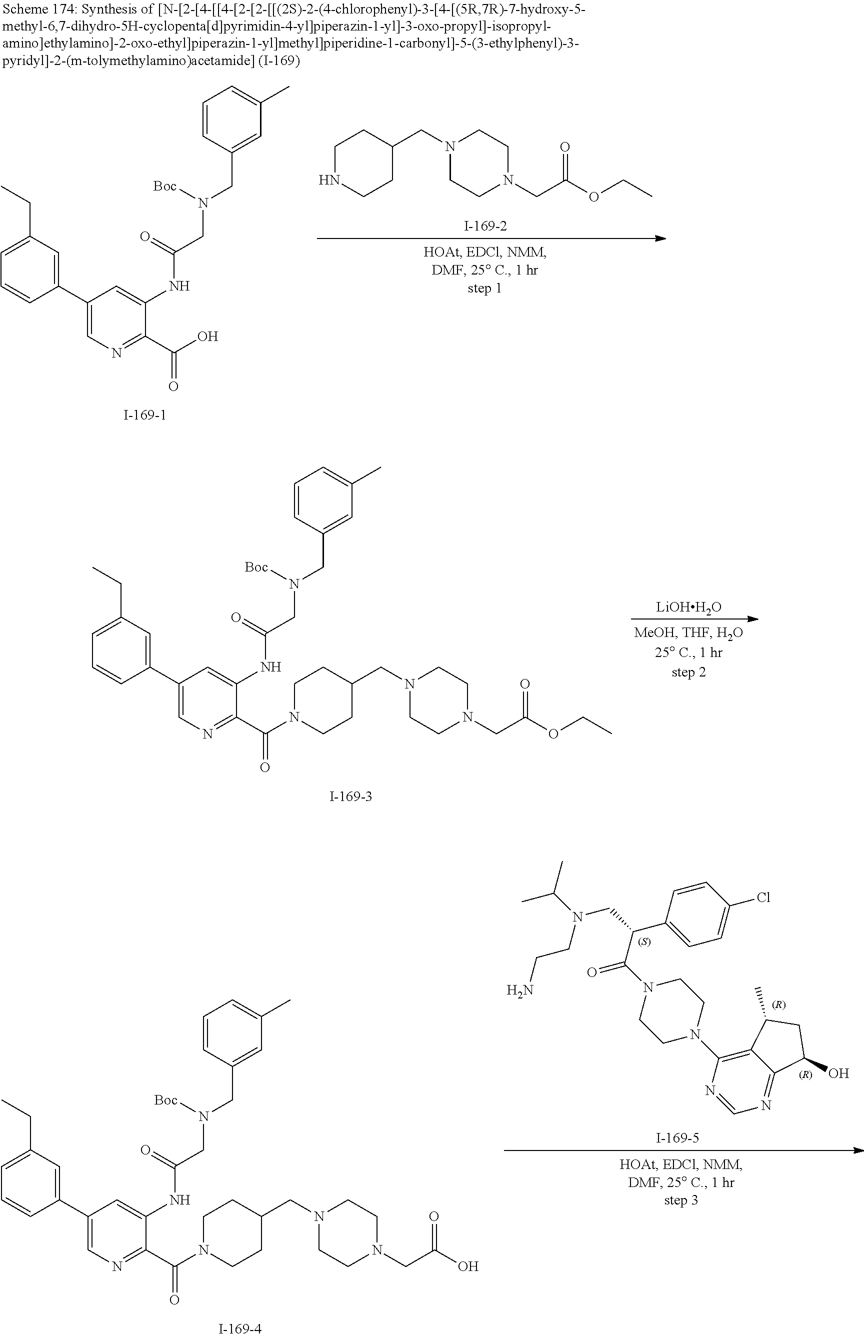

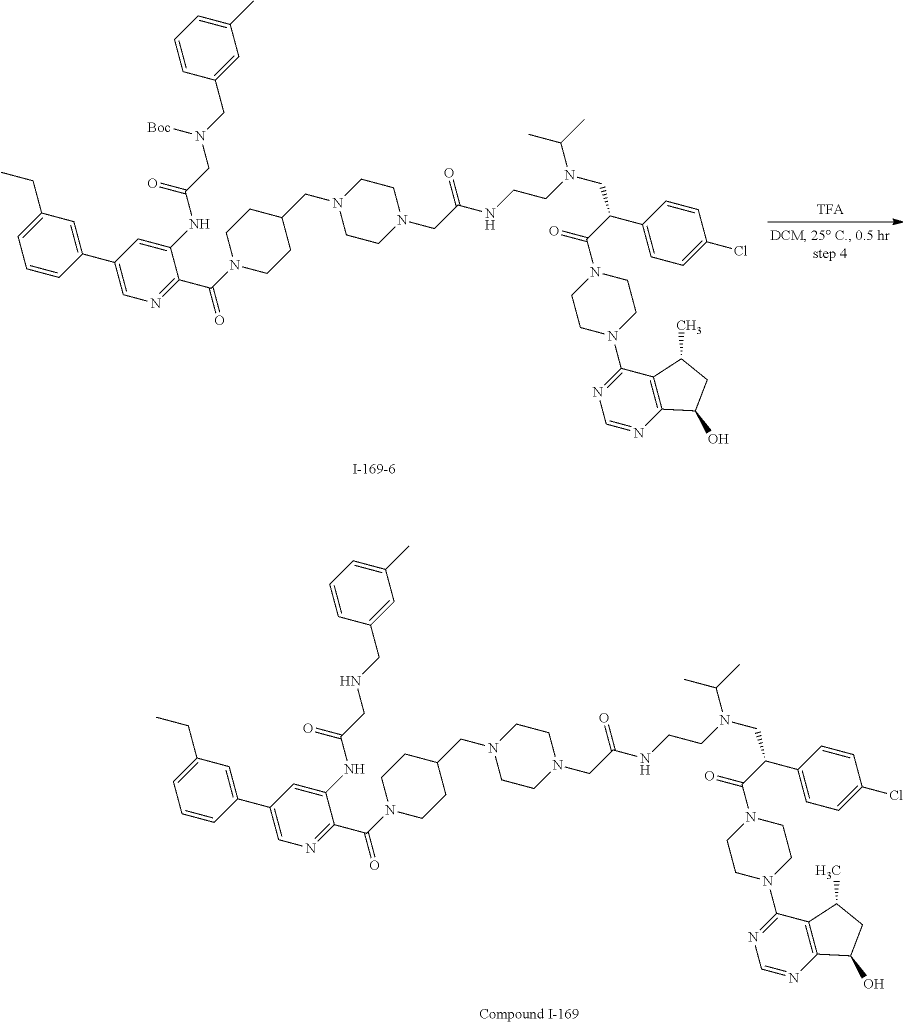 [SMILES: CCc1cccc(-c2cnc(C(=O)N3CCC(CN4CCN(CC(=O)NCCN(C[C@@H](C(=O)N5CCN(c6ncnc7c6[C@H](C)C[C@H]7O)CC5)c5ccc(Cl)cc5)C(C)C)CC4)CC3)c(NC(=O)CN(Cc3cccc(C)c3)C(=O)OC(C)(C)C)c2)c1 ; CCc1cccc(-c2cnc(C(=O)N3CCC(CN4CCN(CC(=O)NCCN(C[C@@H](C(=O)N5CCN(c6ncnc7c6[C@H](C)C[C@H]7O)CC5)c5ccc(Cl)cc5)C(C)C)CC4)CC3)c(NC(=O)CNCc3cccc(C)c3)c2)c1]

### Step 1: Synthesis of I-169-3.

To a solution of I-169-1 (100 mg, 198.57 μmol, 1 eq) in DMF (1 mL) was added I-169-2 (53.49 mg, 174.90 μmol, 8.81e-1 eq, HCl) and EDCI (114.20 mg, 595.72 μmol, 3 eq), HOAt (27.03 mg, 198.57 μmol, 27.78 μL, 1 eq) and NMM (100.43 mg, 992.87 μmol, 109.16 μL, 5 eq). The mixture was stirred at 25° C. for 1 hr. The reaction mixture was quenched by addition H₂O (1 mL), and extracted with DCM 1.5 mL (0.5 mL*3). The combined organic layers were dried over Na₂SO₄, filtered and concentrated under reduced pressure to give a residue. The residue was purified by flash silica gel chromatography (ISCO®; 12 g SepaFlash® Silica Flash Column, Eluent of 0˜100% Dichloromethane: Methanol ether gradient @ 40 mL/min, DCM/MeOH=10/1, Rf=0.6) and the eluent was concentrated to give product. I-169-3 (40 mg, 48.76 μmol, 24.55% yield, 92.02% purity) was obtained as a yellow solid. LCMS (Method D): R^{t}=0.451 min, (M+H)=755.4.

### Step 2: Synthesis of I-169-4.

To a solution of I-169-3 (30 mg, 39.74 μmol, 1 eq) in H₂O (0.1 mL), THF (0.1 mL) and MeOH (0.1 mL) was added LiOH·H₂O (5.00 mg, 119.21 μmol, 3 eq). The mixture was stirred at 25° C. for 1 hr. The mixture was adjusted the pH to acid with 1M HCl and concentrated under reduced pressure to give a crude product. No purification, the crude product was used to next step. I-169-4 (30 mg, crude) was obtained as a white solid. LCMS (Method D): R^{t}=0.481 min, (M+H)=727.8.

### Step 3: Synthesis of I-169-6.

To a solution of I-169-4 (30 mg, 41.27 μmol, 1 eq) in DMF (0.3 mL) was added I-169-5 (25.39 mg, 41.27 μmol, 1 eq, TFA), EDCI (23.74 mg, 123.81 μmol, 3 eq), HOAt (5.62 mg, 41.27 μmol, 5.77 μL, 1 eq) and NMM (20.87 mg, 206.36 μmol, 22.69 μL, 5 eq). The mixture was stirred at 25° C. for 1 hr. The mixture was concentrated under reduced pressure to give a crude product. The crude product was purified by reverse-phase HPLC (0.1% NH₃·H₂O condition) and lyophilized to give the product. I-169-6 (20 mg, 16.53 μmol, 40.05% yield, 100% purity) was obtained as a white solid. LCMS (Method D): R^{t}=0.898 min, (M+H)=1209.7.

### Step 4: Synthesis of I-169

To a solution of I-169-6 (20 mg, 16.53 μmol, 1 eq) in DCM (0.5 mL) was added TFA (153.50 mg, 1.35 mmol, 0.1 mL). The mixture was stirred at 25° C. for 0.5 hr. The mixture was dried with N₂ to give a crude product. The crude product was purified by reverse-phase HPLC (0.1% TFA condition) and lyophilized to give a crude product. I-169 (8.38 mg, 6.63 μmol, 40.14% yield, 96.896% purity, TFA) was obtained as a white solid. LCMS (Method D): R^{t}=0.303 min, (M+H)=1109.5. SFC: R^{t}=1.407 min, 1.701 min. ¹H NMR (400 MHZ, METHANOL-d4) δ=8.67 (s, 1H), 8.58 (s, 1H), 8.31 (s, 1H), 7.53-7.45 (m, 4H), 7.42 (t, J=8.4 Hz, 3H), 7.39-7.35 (m, 2H), 7.34-7.29 (m, 3H), 5.30 (t, J=8.0 Hz, 1H), 4.78-4.71 (m, 1H), 4.70-4.64 (m, 1H), 4.30-4.18 (m, 3H), 4.00 (s, 2H), 3.97-3.79 (m, 8H), 3.71-3.61 (m, 3H), 3.53-3.38 (m, 5H), 3.27-3.09 (m, 7H), 2.99-2.84 (m, 4H), 2.78-2.72 (m, 2H), 2.40 (s, 3H), 2.32-2.16 (m, 3H), 2.02-1.92 (m, 1H), 1.84-1.74 (m, 1H), 1.65-1.56 (m, 1H), 1.52-1.42 (m, 3H), 1.41-1.32 (m, 5H), 1.32-1.25 (m, 5H), 1.23-1.14 (m, 3H), ¹⁹F NMR (376 MHz, METHANOL-d4) δ=−76.949.

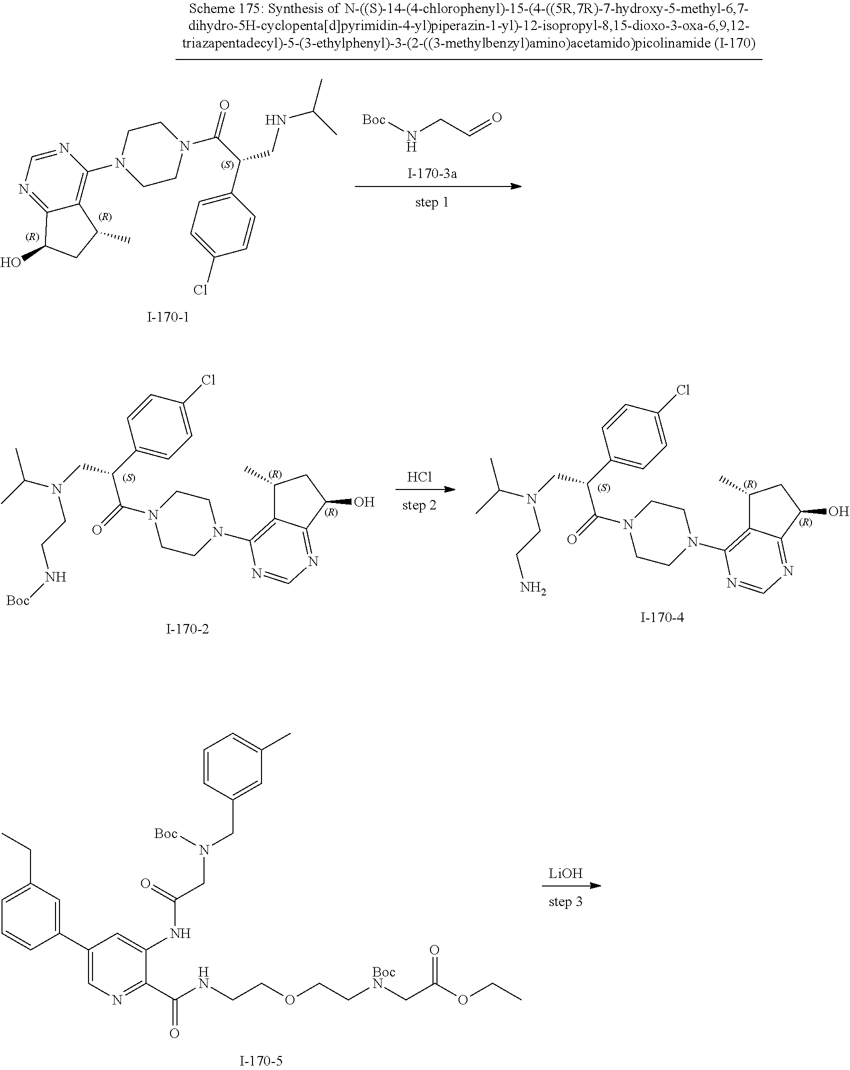

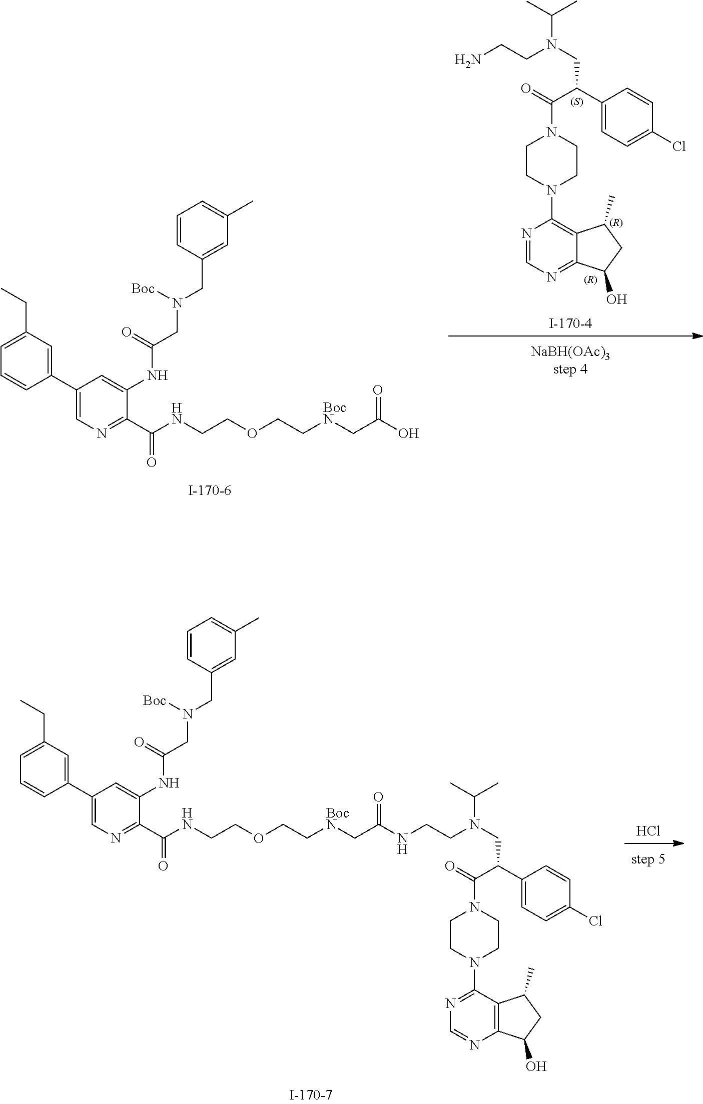

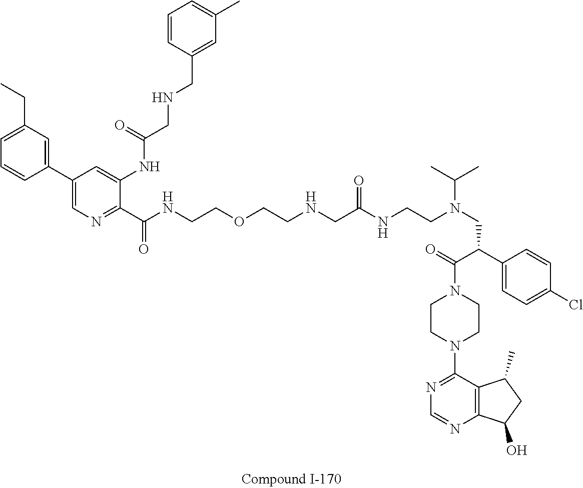 [SMILES: CCc1cccc(-c2cnc(C(=O)NCCOCCNCC(=O)NCCN(C[C@@H](C(=O)N3CCN(c4ncnc5c4[C@H](C)C[C@H]5O)CC3)c3ccc(Cl)cc3)C(C)C)c(NC(=O)CNCc3cccc(C)c3)c2)c1]

### Step 1: Synthesis of I-170-3

To a solution of I-170-1 (100 mg, 218.34 μmol, 1 eq). I-170-3a (139.03 mg, 873.37 μmol, 4 eq), tert-butyl N-(2-oxoethyl) carbamate (139.03 mg, 873.37 μmol, 4 eq) in MeOH (1 mL) and AcOH (13.11 mg, 218.34 μmol, 12.50 μL, 1 eq) was stirred at 25° C. for 0.5 hr. then was added NaBH₃CN (68.61 mg, 1.09 mmol, 5 eq). The mixture was stirred at 60° C. for 36 hr. The reaction mixture was added H₂O (5 mL) and then extracted with DCM (10 mL*3), the combined organic phase was washed with dried by Na₂SO₄, filtered and concentrated. The crude product was purified by reverse-phase HPLC (0.1% FA condition), the eluent was concentrated to remove ACN and lyophilized to get product. I-170-3 (100 mg, 166.34 μmol, 76.18% yield, N/A purity) was obtained as a white solid. LCMS (Method D): R^{t}=0.292 min, (M+H)=601.3. SFC: R^{t}=1.356 min

### Step 2: Synthesis of I-170-4

To a solution of I-170-2 (80 mg, 133.07 μmol, 1 eq) in DCM (0.6 mL) was added TFA (460.50 mg, 4.04 mmol, 0.3 mL, 30.35 eq). The mixture was stirred at 25° C. for 1 hr. The reaction mixture was concentrated under reduced pressure to give a residue. The crude product was purified by reverse-phase HPLC (0.1% TFA condition), the eluent was concentrated to remove ACN and lyophilized to get product. I-170-4 (80 mg, 130.06 μmol, 97.74% yield, N/A purity; TFA) was obtained as a white solid. LCMS (Method D): R^{t}=0.201 min, (M+H)=501.2. SFC: R^{t}=1.499 min.

### Step 3: Synthesis of I-170-6

To a solution of I-170-5 (80 mg, 103.10 μmol, 1 eq) in THF (0.3 mL) and MeOH (0.3 mL) and H₂O (0.3 mL) was added LiOH·H₂O (8.65 mg, 206.20 μmol, 2 eq). The mixture was stirred at 25° C. for 1 hr. The pH of aqueous phase was adjusted to 6 with HCl (1N), then diluted with H₂O (2 mL) and then extracted with DCM (2 mL*3), the combined organic phase was washed with dried by Na₂SO₄, filtered and concentrated. The crude was used next step. I-170-6 (80 mg, crude) was obtained as a white solid. LCMS (Method D): R^{t}=0.528 min, (M+H)=748.4.

### Step 4: Synthesis of I-170-7

To a solution of I-170-6 (72.95 mg, 97.55 μmol, 1 eq), I-170-(60 mg, 97.55 μmol, 1 eq, TFA) in DMF (0.6 mL) was added EDCI (37.40 mg, 195.09 μmol, 2 eq) and HOAt (13.28 mg, 97.55 μmol, 13.65 μL, 1 eq) and NMM (49.33 mg, 487.74 μmol, 53.62 μL, 5 eq). The mixture was stirred at 25° C. for 1 hr. The reaction mixture was added H₂O (2 mL) and then extracted with DCM (2 mL*3), the combined organic phase was washed with dried by Na₂SO₄, filtered and concentrated. The crude product was purified by reverse-phase HPLC (0.1% TFA condition), the eluent was concentrated to remove ACN and lyophilized to get product. I-170-7 (70 mg, 56.87 μmol, 58.30% yield, N/A purity) was obtained as a white solid. LCMS

(Method D): R^{t}=0.451 min, (M+H)/2=616.4.

### Step 5: Synthesis of I-170

To a solution of I-170-7 (60 mg, 48.74 μmol, 1 eq) in DCM (0.6 mL) was added TFA (307.00 mg, 2.69 mmol, 0.2 mL, 55.24 eq). The mixture was stirred at 25° C. for 1 hr. The reaction mixture was concentrated under reduced pressure to give a residue. The crude product was purified by reverse-phase HPLC (0.1% TFA condition), the eluent was concentrated to remove ACN and lyophilized to get product. I-170 (29.5 mg, 25.43 μmol, 52.17% yield, 98.686% purity, TFA) was obtained as a white solid. LCMS (Method D): R^{t}=0.380 min, (M+H)=1030.5. SFC: R^{t}=0.961 min ¹H NMR (400 MHZ, METHANOL-d4) δ=9.23 (d, J=2.0 Hz, 1H), 8.64 (d, J=2.0 Hz, 1H), 8.54 (s, 1H), 7.52 (s, 1H), 7.51-7.41 (m, 4H), 7.40-7.26 (m, 7H), 5.27 (t, J=7.6 Hz, 1H), 4.30 (s, 2H), 4.18 (s, 3H), 3.95 (s, 2H), 3.94-3.87 (m, 3H), 3.87-3.81 (m, 3H), 3.79-3.73 (m, 4H), 3.72-3.66 (m, 4H), 3.66-3.54 (m, 2H), 3.51-3.38 (m, 4H), 3.37-3.32 (m, 4H), 2.77-2.72 (m, 2H), 2.39 (s, 3H), 2.31-2.23 (m, 1H), 2.21-2.10 (m, 1H), 1.48-1.37 (m, 3H), 1.34-1.27 (m, 6H), 1.15 (d, J=7.2 Hz, 3H).

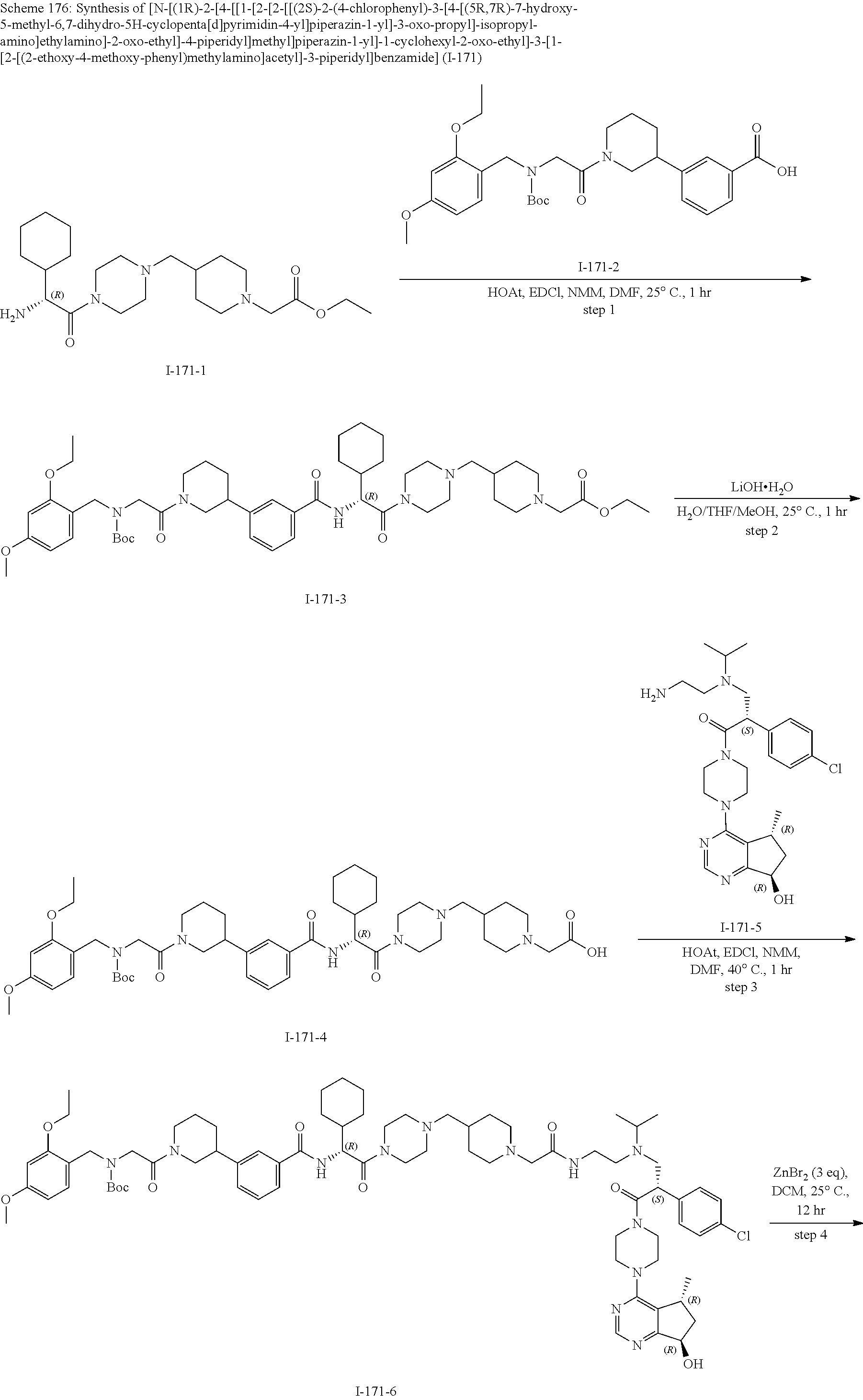

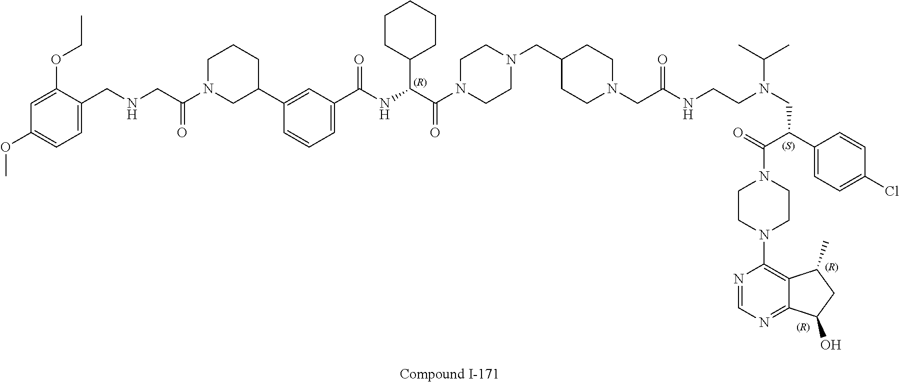

### Step 1: Synthesis of I-171-3.

To a solution of I-171-1 (200 mg, 489.50 μmol, 1 eq) in DMF (2 mL) was added I-171-2 (257.78 mg, 489.50 μmol, 1 eq), EDCI (281.52 mg, 1.47 mmol, 3 eq), HOAt (66.63 mg, 489.50 μmol, 68.48 μL, 1 eq) and NMM (247.56 mg, 2.45 mmol, 269.09 μL, 5 eq). The mixture was stirred at 25° C. for 1 hr. The mixture was concentrated under reduced pressure to give a crude product. The crude product was purified by flash silica gel chromatography (ISCO®; 20 g SepaFlash® Silica Flash Column, Eluent of 0˜100% Dichloromethane: Methanol ether gradient @ 45 mL/min, (PE/EA=1/1, Rf=0.35) and the eluent was concentrated to give product. 10 mg of the product was purified by prep-HPLC (column: Phenomenex luna C18 150*25 mm*10 um; mobile phase: [water (FA)-ACN]; gradient: 23%-53% B over 10 min) and lyophilizated to give the product for SFC. I-171-3 (250 mg, 224.34 μmol, 45.83% yield, 82.304% purity) was obtained as a yellow solid. LCMS (Method D): R^{t}=0.450 min, (M+H)=917.6. SFC: R^{t}=1.650 min, 1.828 min.

### Step 2: Synthesis of I-171-4.

To a solution of I-171-3 (220 mg, 239.87 μmol, 1 eq) in H₂O (0.7 mL), THF (0.7 mL) and MeOH (0.7 mL) was added LiOH·H₂O (30.20 mg, 719.60 μmol, 3 eq). The mixture was stirred at 25° C. for 1 hr. The mixture was adjusted the pH to acid with 1M HCl and concentrated under reduced pressure to give a crude product. No purification, the crude product was used to next step. I-171-4 (220 mg, crude) was obtained as a yellow solid. LCMS (Method D): R^{t}=0.452 min, (M+H)=889.6.

### Step 3: Synthesis of I-171-6.

To a solution of I-171-4 (100 mg, 112.47 μmol, 1 eq) in DMF (1 mL) was added I-171-5 (69.18 mg, 112.47 μmol, 1 eq, TFA), EDCI (64.68 mg, 337.41 μmol, 3 eq), HOAt (15.31 mg, 112.47 μmol, 15.73 μL, 1 eq) and NMM (56.88 mg, 562.35 μmol, 61.83 μL, 5 eq). The mixture was stirred at 25° C. for 1 hr. The mixture was concentrated under reduced pressure to give a crude product. The crude product was purified by reverse-phase HPLC (0.1% NH₃·H₂O condition) and lyophilized to give the product. I-171-6 (60 mg, 41.55 μmol, 36.94% yield, 95.014% purity) was obtained as a white solid. LCMS (Method D): R^{t}=0.871 min, (M+H)=1372.1.

### Step 4: Synthesis of I-171

To a solution of I-171-6 (50 mg, 36.44 μmol, 1 eq) in DCM (0.5 mL) was added dibromozinc (24.62 mg, 109.32 μmol, 5.47 μL, 3 eq). The mixture was stirred at 40° C. for 1 hr. The mixture was washed by MeOH (0.5 mL), filtered and concentrated under reduced pressure to give a crude product. The crude product was purified by reverse-phase HPLC (0.1% FA condition) and lyophilized to give the product. I-171 (13.65 mg, 10.25 μmol, 28.14% yield, 99.021% purity, FA) was obtained as a white solid. LCMS (Method D): R^{t}=0.465 min, (M+H)=1271.8. SFC: R^{t}=2.574 min, 2.745 min. ¹H NMR (400 MHZ, METHANOL-d4) δ=8.42 (s, 2H), 7.80-7.68 (m, 2H), 7.53-7.43 (m, 2H), 7.39 (s, 4H), 7.29 (t, J=8.4 Hz, 1H), 6.65-6.53 (m, 2H), 4.98 (t, J=6.8 Hz, 1H), 4.58-4.36 (m, 2H), 4.24-4.10 (m, 4H), 4.08-3.89 (m, 3H), 3.87-3.64 (m, 12H), 3.63-3.43 (m, 4H), 3.39-3.25 (m, 3H), 3.27-3.16 (m, 4H), 3.16-2.92 (m, 3H), 2.91-2.67 (m, 5H), 2.59-2.39 (m, 6H), 2.29 (d, J=6.8 Hz, 2H), 2.18-2.03 (m, 3H), 1.94-1.65 (m, 12H), 1.60-1.40 (m, 4H), 1.40-1.17 (m, 6H), 1.14-1.02 (m, 10H).

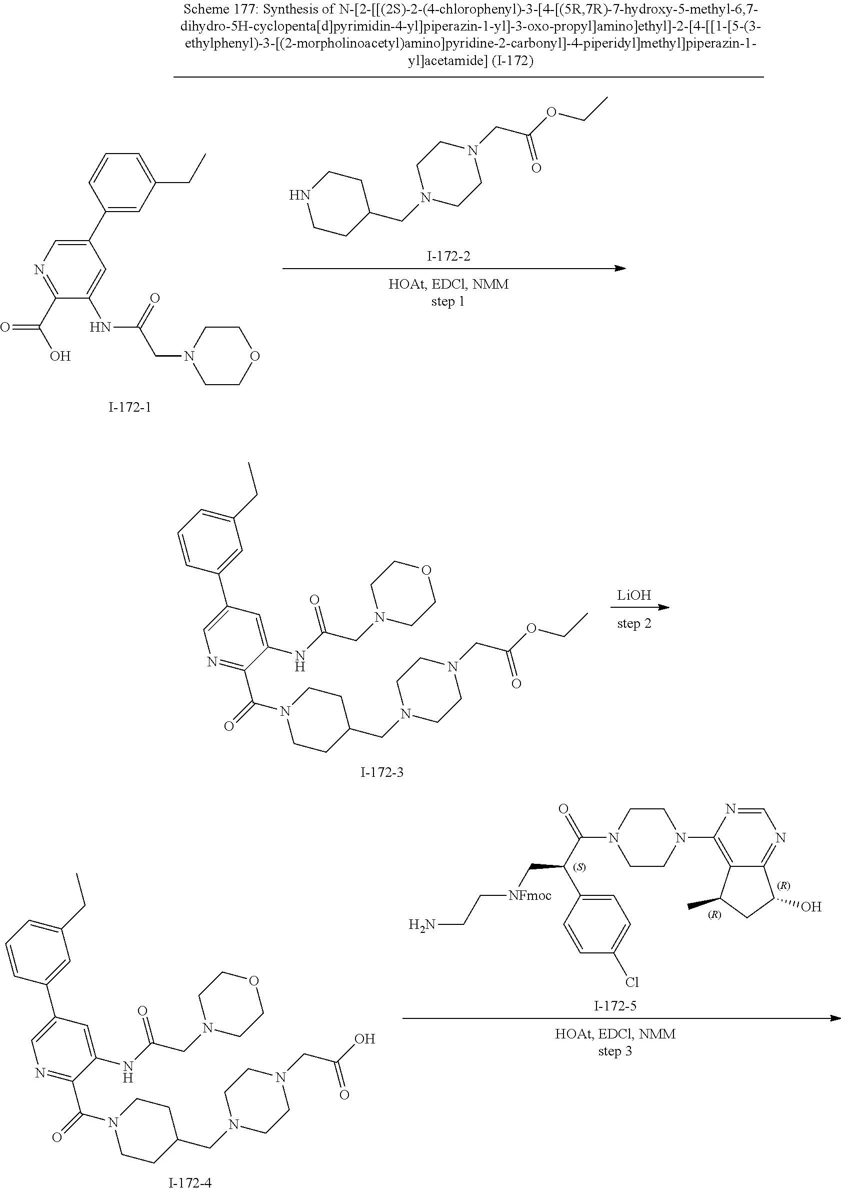

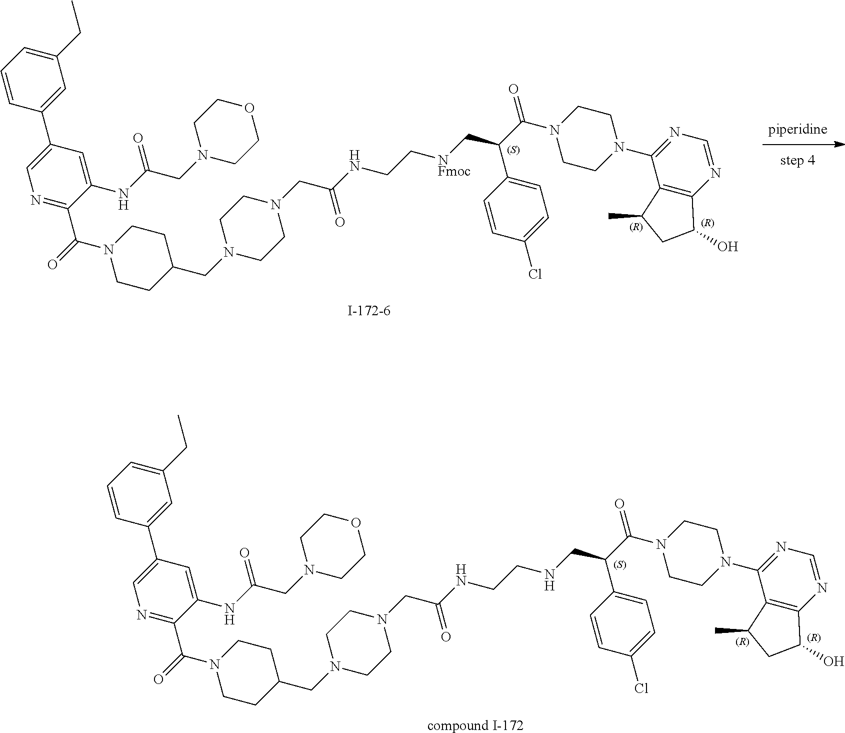

### Step 1: Synthesis of I-172-3

To a solution of I-172-1 (130 mg, 351.91 μmol, 1 eq) in DMF (1.5 mL) was added EDCI (202.38 mg, 1.06 mmol, 3 eq), NMM (177.97 mg, 1.76 mmol, 193.45 μL, 5 eq), I-172-2 (94.80 mg, 351.91 μmol, 1 eq) and HOAt (47.90 mg, 351.91 μmol, 49.23 μL, 1 eq). The mixture was stirred at 60° C. for 2 h. Without workup. The crude product was purified by prep-HPLC (0.1% NH₃·H₂O condition) and the eluent was lyophilized to give I-172-3 (180 mg, 269.66 μmol, 76.63% yield, 93% purity) was obtained as a yellow oil. LCMS (Method D): R^{t}=0.334 min, (M+H)=621.8.

### Step 2: Synthesis of I-172-4

The I-172-3 (180 mg, 289.96 μmol, 1 eq) in MeOH (0.6 mL), H₂O (0.6 mL) and THF (0.6 mL) was added LiOH H₂O (36.50 mg, 869.87 μmol, 3 eq). The mixture was stirred at 25° C. for 0.5 h. The mixture was adjusted to pH 5˜6 with 1M HCl, and concentrated under reduced pressure to give I-172-4 (180 mg, crude) was obtained as a yellow solid. LCMS (Method D): R^{t}=0.306 min, (M+H)=593.3.

### Step 3: Synthesis of I-172-6

To a solution of I-172-4 (41.29 mg, 69.67 μmol, 1 eq) in DMF (0.5 mL) was added EDCI (40.07 mg, 209.01 μmol, 3 eq), NMM (35.23 mg, 348.34 μmol, 38.30 μL, 5 eq), I-172-5 (50 mg, 69.67 μmol, 1 eq, HCl salt) and HOAt (9.48 mg, 69.67 μmol, 9.75 μL, 1 eq). The mixture was stirred at 25° C. for 1 h. The reaction mixture was washed with H₂O (2 mL) at 25° C., and mixture was extracted with EA 6 ml (2 ml*3), and combined organic phase was dried with anhydrous sodium sulfate, filtered and filtrate was concentrated to give I-172-6 (50 mg, crude) as a yellow solid. LCMS (Method D): R^{t}=0.432 min, (M/2+H)=628.8.

### Step 4: Synthesis of I-172

To a solution of I-172-6 (50 mg, 39.81 μmol, 1 eq) in DMF (0.5 mL) was added piperidine (10.17 mg, 119.43 μmol, 11.79 μL, 3 eq). The mixture was stirred at 25° C. for 0.5 h. Without workup. The crude product was purified by prep-HPLC (0.1% FA condition) and the eluent was lyophilized to give crude product. The crude product was second purified by prep-HPLC (column: CD07-Daisogel SP-100-8-ODS-PK 150*25*10 um; mobile phase: [water (NH4HCO3)-ACN]; gradient: 32%-62% B over 15 min) and the eluent was lyophilized to give I-172 (6.88 mg, 6.09 μmol, 15.29% yield, 91.472% purity) was obtained as a white solid. SFC: R^{t}=0.927 min. LCMS (Method D): R^{t}=0.295 min, (M+H)=1033.3. ¹H NMR (400 MHz, METHANOL-d4) δ=8.88 (d, J=1.6 Hz, 1H), 8.60 (d, J=1.6 Hz, 1H), 8.41 (s, 1H), 7.56-7.47 (m, 2H), 7.43 (t, J=7.6 Hz, 1H), 7.40-7.35 (m, 2H), 7.35-7.28 (m, 3H), 4.96 (t, J=6.8 Hz, 1H), 4.74 (d, J=13.2 Hz, 1H), 4.21-4.18 (m, 1H), 3.92-3.86 (m, 1H), 3.84-3.80 (m, 4H), 3.76-3.67 (m, 2H), 3.66-3.57 (m, 2H), 3.56-3.44 (m, 3H), 3.37-3.33 (m, 2H), 3.29-3.23 (m, 2H), 3.20 (s, 2H), 3.17-3.11 (m, 1H), 3.02-2.88 (m, 3H), 2.83-2.68 (m, 5H), 2.62 (s, 4H), 2.53 (3, 7H), 2.30-2.23 (m, 2H), 2.18-2.00 (m, 3H), 1.99-1.87 (m, 2H), 1.84-1.64 (m, 2H), 1.33-1.21 (m, 5H), 1.11 (d, J=6.8 Hz, 3H).

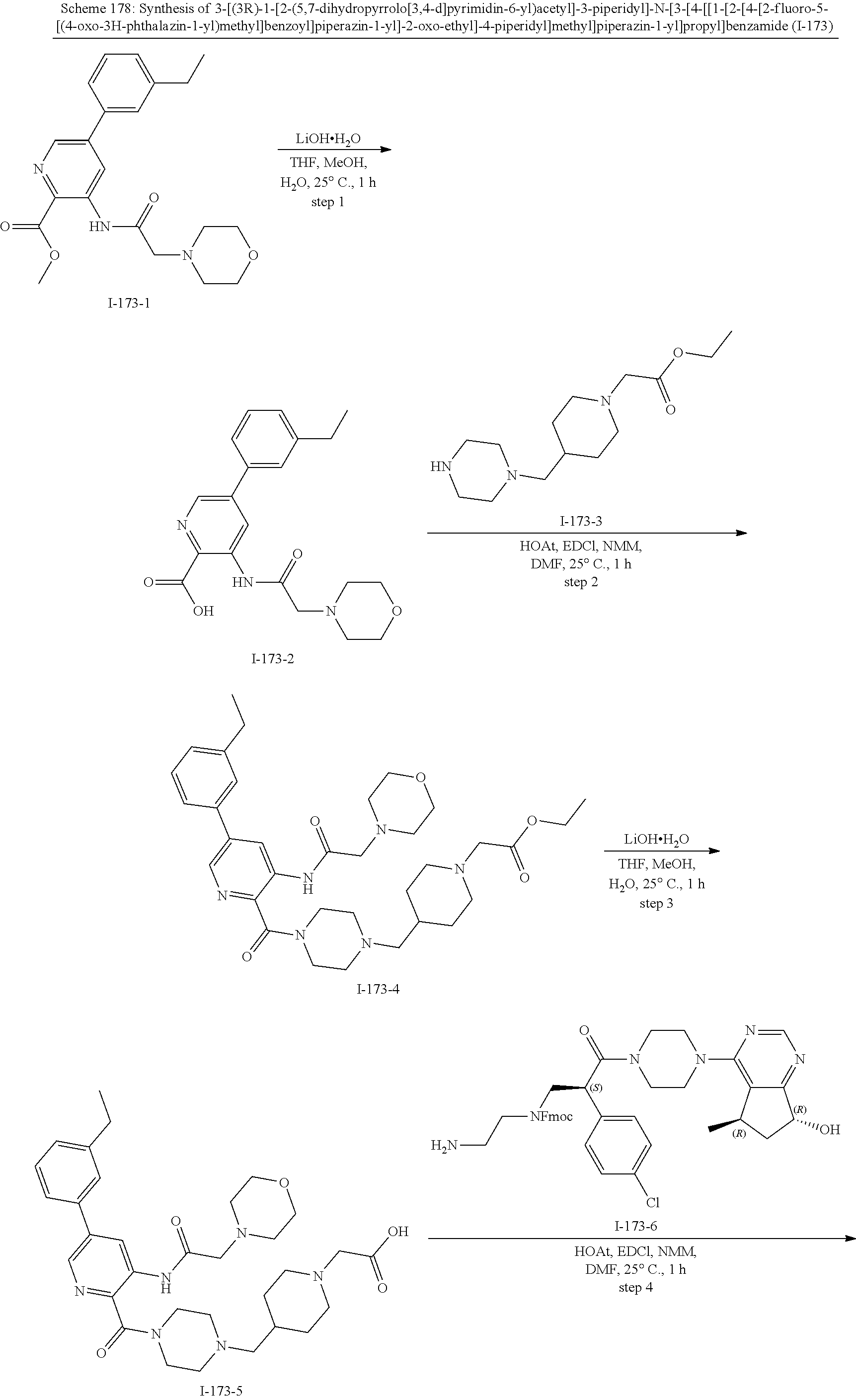

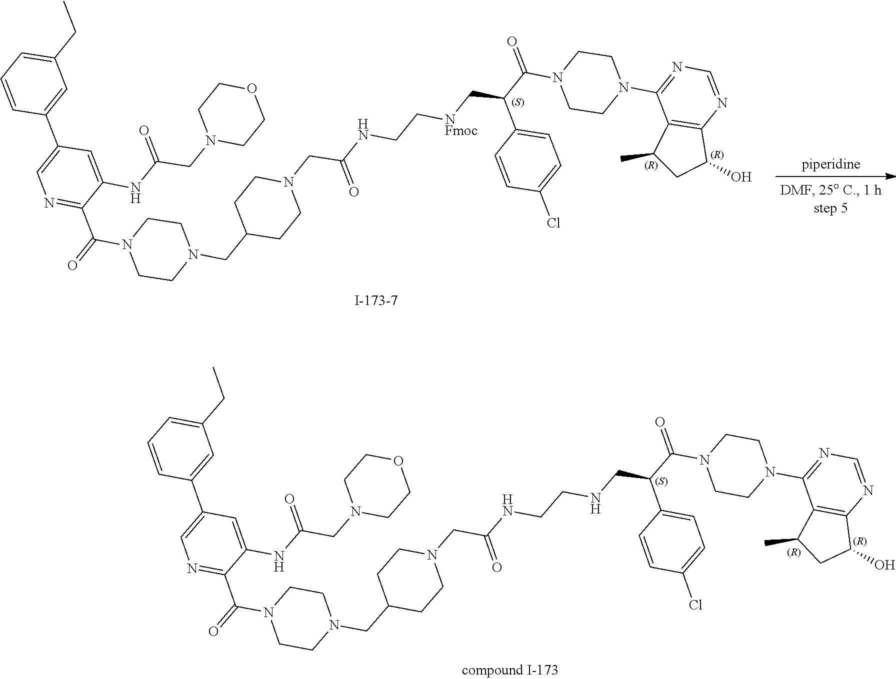

### Step 1: Synthesis of I-173-2

To a solution of I-173-1 (310 mg, 808.47 μmol, 1 eq) in THF (1.5 mL), MeOH (1.5 mL) and H₂O (1.2 mL) was added LiOH H₂O (169.63 mg, 4.04 mmol, 5 eq). The mixture was stirred at 25° C. for 1 hr. The reaction mixture was concentrated under reduced pressure to give a residue. The crude product was used into the next step without further purification. I-173-2 (550 mg, crude) was obtained as a red solid. LCMS (Method D): R^{t}=0.282 min, (M+H)=370.1.

### Step 2: Synthesis of I-173-4

To a solution of I-173-2 (500 mg, 1.35 mmol, 1 eq) and I-173-3 (413.96 mg, 1.35 mmol, 1 eq, HCl slat) in DMF (5 mL) was added HOAt (184.23 mg, 1.35 mmol, 189.34 μL, 1 eq), NMM (684.51 mg, 6.77 mmol, 744.03 μL, 5 eq) and EDCI (778.40 mg, 4.06 mmol, 3 eq). The mixture was stirred at 40° C. for 24 hrs. The reaction mixture was filtered and concentrated under reduced pressure to give a residue. The residue was purified by flash silica gel chromatography (ISCO®; 40 g SepaFlash® Silica Flash Column, Eluent of 0˜70% Ethylacetate/Petroleum ether gradient @, 60 mL/min) and concentrated under reduced pressure to give a residue. I-173-4 (800 mg, 1.20 mmol, 88.55% yield, 93% purity) was obtained as a yellow oil. LCMS (Method D): R^{t}=0.253 min, (M+H)=621.4.

### Step 3: Synthesis of I-173-5

To a solution of I-173-4 (750 mg, 1.21 mmol, 1 eq) in THF (7.5 mL), H₂O (3.75 mL) and MeOH (7.5 mL) was added LiOH H₂O (253.49 mg, 6.04 mmol, 5 eq). The mixture was stirred at 25° C. for 1 hr. The reaction mixture was filtered and concentrated under reduced pressure to give a residue. The crude product was purified by reverse-phase HPLC (neutral condition) followed by lyophilization to give product. I-173-5 (371 mg, 575.85 μmol, 47.66% yield, 92% purity) was obtained as a white solid. LCMS (Method D): R^{t}=0.295 min, (M+H)=593.3.

### Step 4: Synthesis of I-173-7

To a solution of I-173-5 (50 mg, 84.36 μmol, 1 eq) and I-173-6 (57.46 mg, 84.36 μmol, 1 eq) in DMF (0.5 mL) was added EDCI (48.51 mg, 253.07 μmol, 3 eq), NMM (42.66 mg, 421.78 μmol, 46.37 μL, 5 eq) and HOAt (11.48 mg, 84.36 μmol, 11.80 μL, 1 eq). The mixture was stirred at 25° C. for 1 hr. The reaction mixture was filtered and concentrated under reduced pressure to give a residue. The crude product was purified by reverse-phase HPLC (0.1% NH₃·H₂O condition) followed by lyophilization to give product. I-173-7 (33 mg, 26.28 μmol, 31.15% yield) was obtained as a yellow solid. LCMS (Method D): R^{t}=0.786 min, (M+H)=1255.4.

### Step 5: Synthesis of I-173

To a solution of I-173-7 (33 mg, 26.28 μmol, 1 eq) in DMF (0.3 mL) was added piperidine (4.47 mg, 52.55 μmol, 5.19 μL, 2 eq). The mixture was stirred at 25° C. for 1 hr. The reaction mixture was filtered and concentrated under reduced pressure to give a residue. The crude product was purified by reverse-phase HPLC (neutral condition) followed by lyophilization to give product. I-173 (7.09 mg, 6.84 μmol, 26.03% yield, 99.732% purity) was obtained as a yellow solid. LCMS (Method D): R^{t}=0.273 min, (M+H)=1033.3. ¹H NMR (400 MHZ, METHANOL-d4) δ=8.89 (d, J=2.0 Hz, 1H), 8.60 (d, J=2.0 Hz, 1H), 8.41 (s, 1H), 7.55-7.49 (m, 2H), 7.43 (t, J=7.6 Hz, 1H), 7.40-7.35 (m, 2H), 7.34-7.29 (m, 3H), 4.99-4.93 (m, 1H), 4.20-4.19 (m, 1H), 3.92-3.85 (m, 3H), 3.84-3.81 (m, 4H), 3.77-3.66 (m, 2H), 3.66-3.44 (m, 8H), 3.37-3.33 (m, 2H), 3.28-3.23 (m, 2H), 3.21 (s, 2H), 2.97 (s, 2H), 2.84 (d, J=2.4 Hz, 2H), 2.79-2.72 (m, 5H), 2.65-2.60 (m, 4H), 2.56 (s, 2H), 2.46 (d, J=4.4 Hz, 2H), 2.26 (d, J=7.2 Hz, 2H), 2.17-2.09 (m, 3H), 2.08-2.01 (m, 1H), 1.77 (d, J=12.0 Hz, 2H), 1.62-1.53 (m, 1H), 1.29 (t, J=7.6 Hz, 4H), 1.11 (d, J=6.8 Hz, 3H).

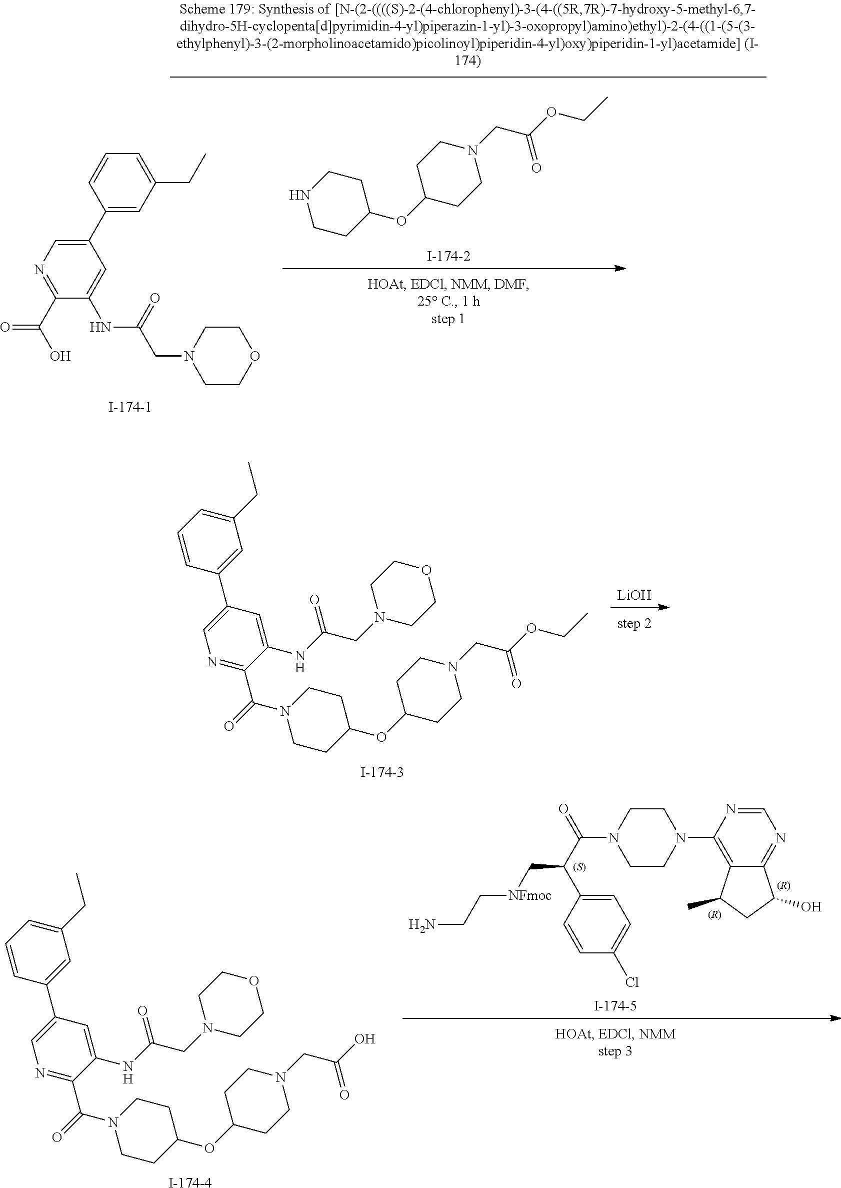

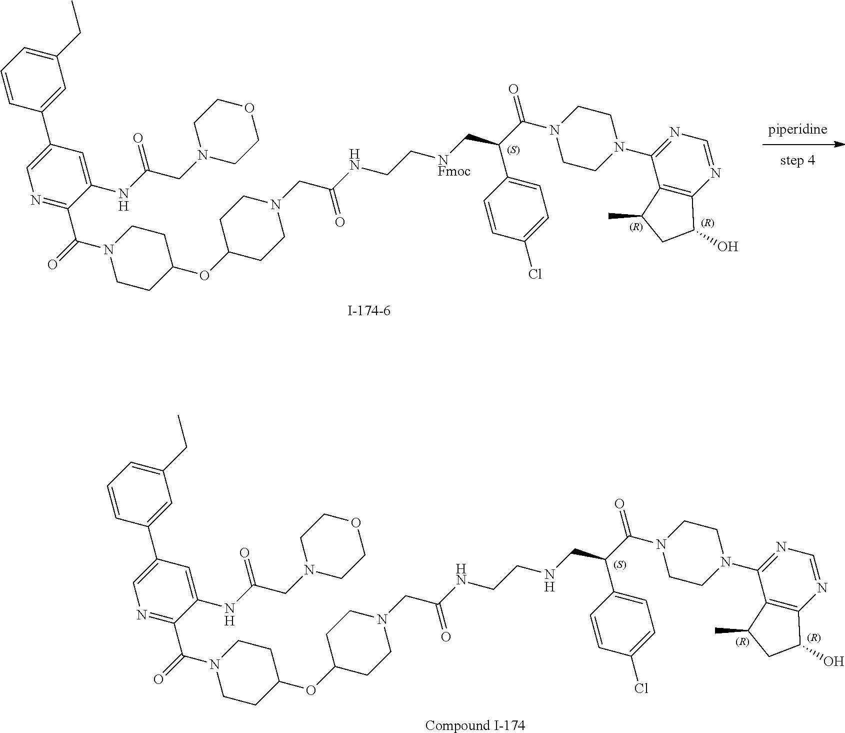

### Step 1: Synthesis of I-174-3

To a solution of I-174-1 (130 mg, 351.91 μmol, 1 eq) and I-174-2 (95.14 mg, 351.91 μmol, 1 eq) in DMF (1.5 mL) was added EDCI (202.38 mg, 1.06 mmol, 3 eq), HOAt (47.90 mg, 351.91 μmol, 49.23 μL, 1 eq) and NMM (177.97 mg, 1.76 mmol, 193.45 μL, 5 eq). The mixture was stirred at 25° C. for 0.5 hr. The reaction mixture was filtered and concentrated under reduced pressure to give a residue. The residue was purified by prep-HPLC (0.1% FA) the eluent was concentrated to remove ACN and lyophilized to get I-174-3 (0.11 g, 174.32 μmol, 49.53% yield) was obtained as a white solid. LCMS (Method D): R^{t}=0.644 min, (M+H)=622.6.

### Step 2: Synthesis of I-174-4

To a solution of I-174-3 (0.1 g, 160.83 μmol, 1 eq) in THF (0.5 mL), MeOH (0.5 mL) and H₂O (0.5 mL) was added LiOH H₂O (20.25 mg, 482.50 μmol, 3 eq). The mixture was stirred at 25° C. for 0.5 hr. The reaction mixture was adjusted pH=6 by HCl (1M) at 25 C, and then concentrated under reduced pressure to give I-174-4 (0.1 g, crude) was obtained as a white solid. LCMS (Method D): R^{t}=0.323 min, (M+H)=594.4.

### Step 3: Synthesis of I-174-6

To a solution of I-174-4 (41.36 mg, 69.67 μmol, 1 eq) and I-174-5 (50 mg, 69.67 μmol, 1 eq, HCl salt) in DMF (1 mL) was added EDCI (40.07 mg, 209.01 μmol, 3 eq), NMM (35.23 mg, 348.34 μmol, 38.30 μL, 5 eq) and HOAt (9.48 mg, 69.67 μmol, 9.75 μL, 1 eq). The mixture was stirred at 25° C. for 0.5 hr. The reaction mixture was extracted with DCM 10 mL (5 mL*2). The combined organic layers were washed with aq. NaCl 6 mL (3 mL*2), dried over Na₂SO₄, filtered and concentrated under reduced pressure to give I-174-6 (0.08 g, 63.65 μmol, 91.36% yield) as a white solid. LCMS (Method D): R^{t}=0.425 min, (M+H)=1256.6.

### Step 4: Synthesis of I-174

To a solution of I-174-6 (0.08 g, 63.65 μmol, 1 eq) in DMF (1 mL) was added piperidine (16.26 mg, 190.94 μmol, 18.86 μL, 3 eq). The mixture was stirred at 25° C. for 1 hr. The reaction mixture was filtered and concentrated under reduced pressure to give a residue. The residue was purified by prep-HPLC (column: CD20-Waters Xbidge BEH C18 250*25*10 um; mobile phase: [water (NH4HCO3)-ACN]; gradient: 37%-67% B over 10 min) the eluent was concentrated to remove ACN and lyophilized to get I-174 (25.09 mg, 23.70 μmol, 37.24% yield, 97.742% purity) was obtained as a white solid. LCMS

(Method D): R^{t}=0.293 min, (M+H)=1034.4. SFC: R^{t}=1.626 min. ¹H NMR (400 MHZ, METHANOL-d4) δ=8.87 (s, 1H), 8.60 (d, J=2.0 Hz, 1H), 8.40 (s, 1H), 7.55-7.49 (m, 2H), 7.45-7.41 (m, 1H), 7.40-7.35 (m, 2H), 7.35-7.29 (m, 3H), 4.98-4.93 (m, 1H), 4.23-4.10 (m, 2H), 3.94-3.86 (m, 1H), 3.84-3.80 (m, 5H), 3.78-3.74 (m, 1H), 3.73-3.66 (m, 2H), 3.65-3.58 (m, 3H), 3.57-3.51 (m, 3H), 3.50-3.38 (m, 2H), 3.36-3.33 (m, 2H), 3.29-3.23 (m, 2H), 3.20 (s, 2H), 2.98 (s, 2H), 2.79-2.71 (m, 7H), 2.65-2.60 (m, 4H), 2.34-2.26 (m, 2H), 2.16-2.08 (m, 1H), 2.07-2.03 (m, 1H), 2.02-1.96 (m, 1H), 1.94-1.85 (m, 3H), 1.71-1.59 (m, 4H), 1.32-1.27 (m, 3H), 1.15-1.08 (m, 3H).

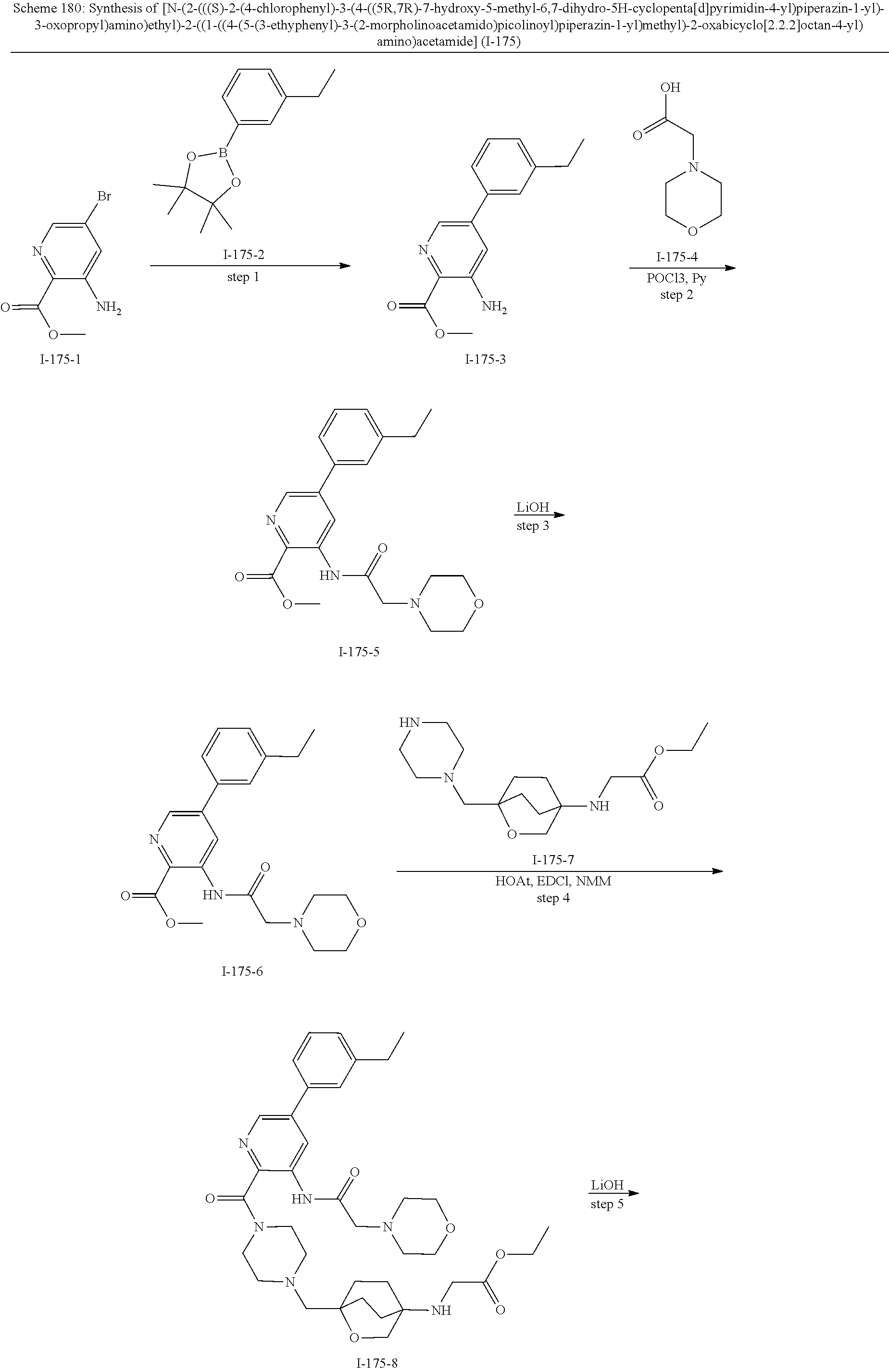

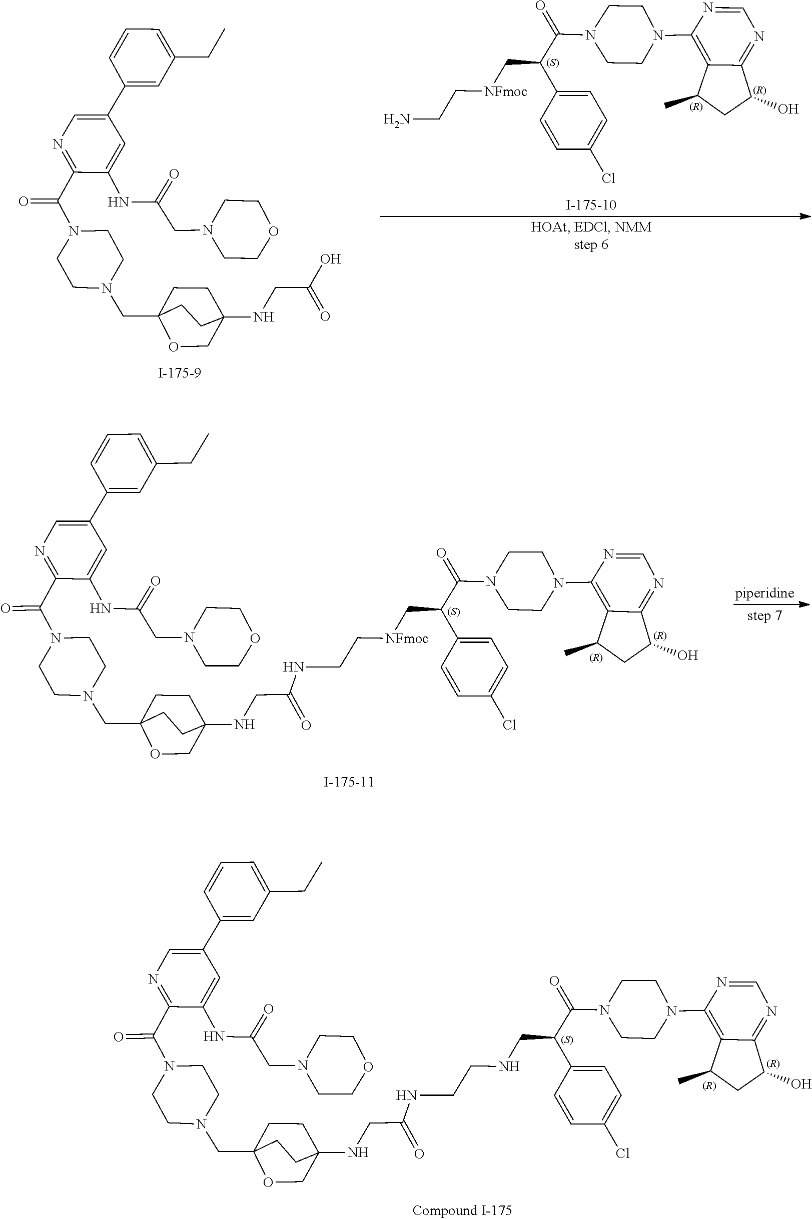

### Step 1: Synthesis of I-175-3

A mixture of I-175-1 (10 g, 43.3 mmol, 1 eq), I-175-2 (7.14 g, 47.61 mmol, 1.1 eg), (dtbpf) PdCl2 (2.82 g, 4.33 mmol, 0.1 eq) and K₃PO₄ (27.56 g, 129.84 mmol, 3 eq) in dioxane (100 mL), H₂O (20 mL) was degassed and purged with N₂ for 3 times, and then the mixture was stirred at 80° C. for 1 hr under N₂ atmosphere. The reaction mixture was diluted with H₂O 100 mL and extracted with DCM 300 mL (100 mL*3). The combined organic layers were washed with NaCl (aq) 50 mL, dried over Na₂SO₄, filtered and concentrated under reduced pressure to give a residue. The residue was purified by flash silica gel chromatography (ISCO®; 220 g SepaFlash® Silica Flash Column, Eluent of 0˜100% Ethyl acetate/Petroleum ether gradient (80 mL/min) was concentrated under reduced pressure to give I-175-3 (10 g, 38.93 mmol, 89.95% yield, 99.781% purity) as a yellow solid. LCMS (Method D): R^{t}=0.430 min, (M+H)=257.1.

### Step 2: Synthesis of I-175-5

To a solution of I-175-3 (0.5 g, 1.95 mmol, 1 eq) and I-175-4 (311.49 mg, 2.15 mmol, 1.1 eq) in DMF (5 mL) was added EDCI (1.12 g, 5.85 mmol, 3 eq) and HOAt (265.53 mg, 1.95 mmol, 272.90 μL, 1 eq), NMM (986.61 mg, 9.75 mmol, 1.07 mL, 5 eq). The mixture was stirred at 25° C. for 1 hr. The reaction mixture was filtered and concentrated under reduced pressure to give a residue. The residue was purified by prep-HPLC (column: CD19-Daisogel SP-100-8-ODS-PK 200*50*10 um; mobile phase: [water (NH3H2O)-ACN]; gradient: 46%-76% B over 11 min) the eluent was concentrated to remove ACN and lyophilized to get I-175-5 (0.2 g, 521.59 μmol, 26.74% yield) was obtained as a yellow solid. LCMS (Method D): R^{t}=0.578 min, (M+H)=257.0.

### Step 3: Synthesis of I-175-6

To a solution of methyl I-175-5 (0.2 g, 521.59 μmol, 1 eq) in THF (1 mL), MeOH (1 mL) and H₂O (1 mL) was added LiOH·H₂O (65.66 mg, 1.56 mmol, 3 eq). The mixture was stirred at 25° C. for 0.5 hr. The reaction mixture was concentrated under reduced pressure to give I-175-6 (0.19 g, 514.33 μmol, 98.61% yield) as a white solid. LCMS (Method D): R^{t}=0.257 min, (M+H)=370.1.

### Step 4: Synthesis of I-175-8

To a solution of I-175-6 (0.15 g, 406.05 μmol, 1 eq) and I-175-7 (141.26 mg, 406.05 μmol, 1 eq, HCl salt) in DMF (1.5 mL) was added EDCI (233.52 mg, 1.22 mmol, 3 eq), NMM (205.35 mg, 2.03 mmol, 223.21 μL, 5 eq) and HOAt (55.27 mg, 406.05 μmol, 56.80 μL, 1 eq). The mixture was stirred at 25° C. for 0.5 hr. The reaction mixture was filtered and concentrated under reduced pressure to give a residue. The residue was purified by prep-HPLC (0.1% NH3HCO3) the eluent was concentrated to remove ACN and lyophilized to get I-175-8 (150 mg, 226.31 μmol, 55.73% yield) was obtained as a white solid. LCMS (Method D): R^{t}=0.675 min, (M+H)=663.4.

### Step 5: Synthesis of I-175-9

To a solution of I-175-8 (0.13 g, 196.13 μmol, 1 eq) in THF (1 mL), MeOH (1 mL) and H₂O (1 mL) was added LiOH H₂O (24.69 mg, 588.40 μmol, 3 eq). The mixture was stirred at 25° C. for 0.5 hr. The reaction mixture was adjusted pH=6 by HCl (1M), and then filtered and concentrated under reduced pressure to give I-175-9 (0.1 g, 157.54 μmol, 80.32% yield) as a white solid. LCMS (Method D): R^{t}=0.290 min, (M+H)=635.4.

### Step 6: Synthesis of I-175-11

To a solution of I-175-9 (44.22 mg, 69.67 μmol, 1 eq) and I-175-10 (50 mg, 69.67 μmol, 1 eq, HCl salt) in DMF (1 mL) was added EDCI (40.07 mg, 209.01 μmol, 3 eq), HOAt (9.48 mg, 69.67 μmol, 9.75 μL, 1 eq) and NMM (35.23 mg, 348.34 μmol, 38.30 μL, 5 eq). The mixture was stirred at 25° C. for 0.5 hr. The reaction mixture was extracted with DCM 20 mL (10 mL*2). The combined organic layers were washed with NaCl (aq) 10 mL (5 mL*2), dried over Na₂SO₄, filtered and concentrated under reduced pressure to give I-175-10 (80 mg, crude) was obtained as a brown oil. LCMS (Method D): R^{t}=0.407 min, (M/2+H)=649.8.

### Step 7: Synthesis of I-175

To a solution of I-175-11 (0.08 g, 61.63 μmol, 1 eq) in DMF (1 mL) was added piperidine (15.74 mg, 184.90 μmol, 18.26 μL, 3 eq). The mixture was stirred at 25° C. for 0.5 hr. The reaction mixture was filtered and concentrated under reduced pressure to give a residue. The residue was purified by prep-HPLC (column: CD20-Waters Xbidge BEH C18 250*25*10 um; mobile phase: [water (NH4HCO3)-ACN]; gradient: 35%-65% B over 15 min) the eluent was concentrated to remove ACN and lyophilized to get I-175 (9.47 mg, 8.80 μmol, 14.28% yield, 100% purity) was obtained as a yellow solid. LCMS (Method D): R^{t}=0.271 min, (M+H)=1075.4. SFC method: R^{t}=1.081 min. ¹H NMR (400 MHZ, METHANOL-d4) δ=8.89 (d, J=2.0 Hz, 1H), 8.59 (d, J=2.0 Hz, 1H), 8.41 (s, 1H), 7.55-7.48 (m, 2H), 7.44-7.41 (m, 1H), 7.40-7.35 (m, 2H), 7.34-7.29 (m, 3H), 4.98-4.94 (m, 1H), 4.19-4.16 (m, 1H), 3.91-3.77 (m, 8H), 3.75-3.66 (m, 2H), 3.65-3.59 (m, 4H), 3.58-3.45 (m, 6H), 3.29-3.22 (m, 2H), 3.20 (s, 2H), 3.17 (s, 2H), 2.78-2.71 (m, 5H), 2.69-2.65 (m, 2H), 2.64-2.61 (m, 4H), 2.56 (s, 2H), 2.31 (s, 2H), 2.17-2.09 (m, 1H), 2.08-2.03 (m, 1H), 2.01 (s, 2H), 1.74-1.62 (m, 6H), 1.31-1.27 (m, 3H), 1.11 (d, J=6.8 Hz, 3H).

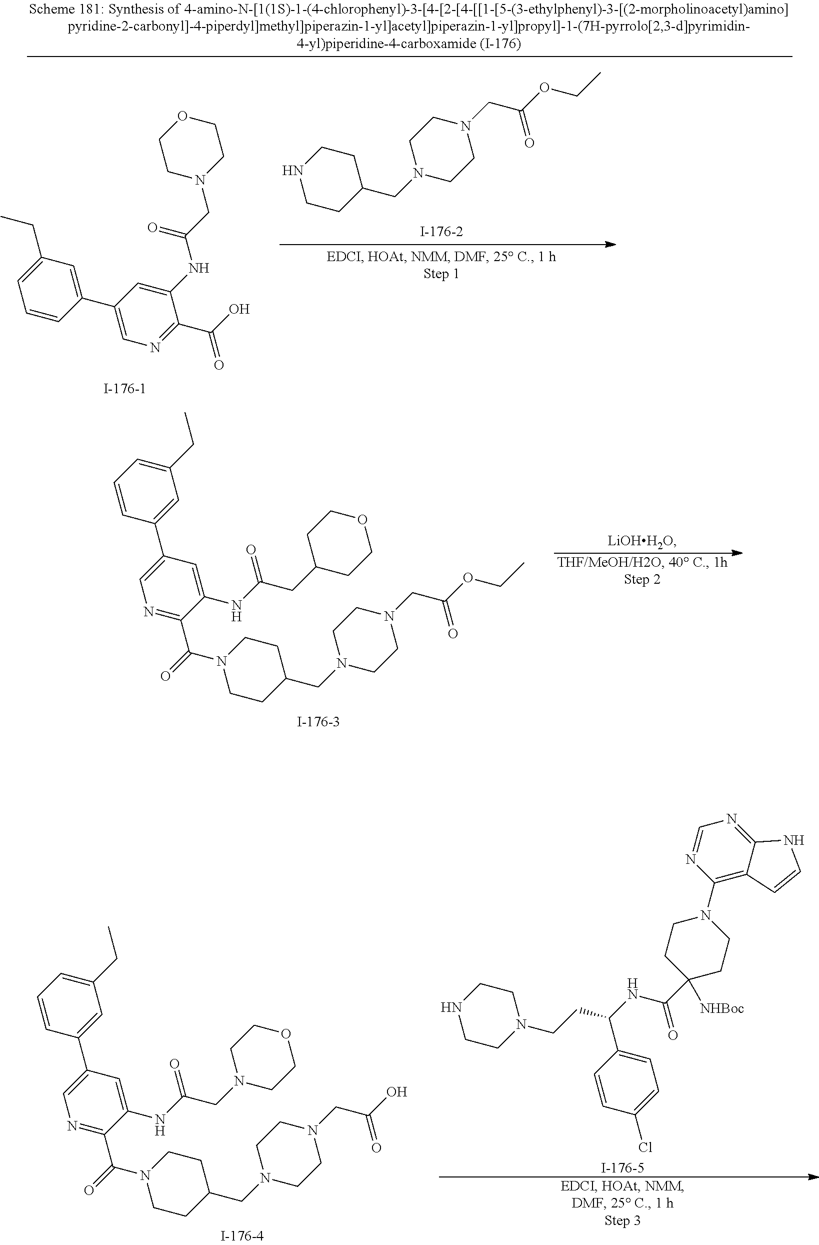

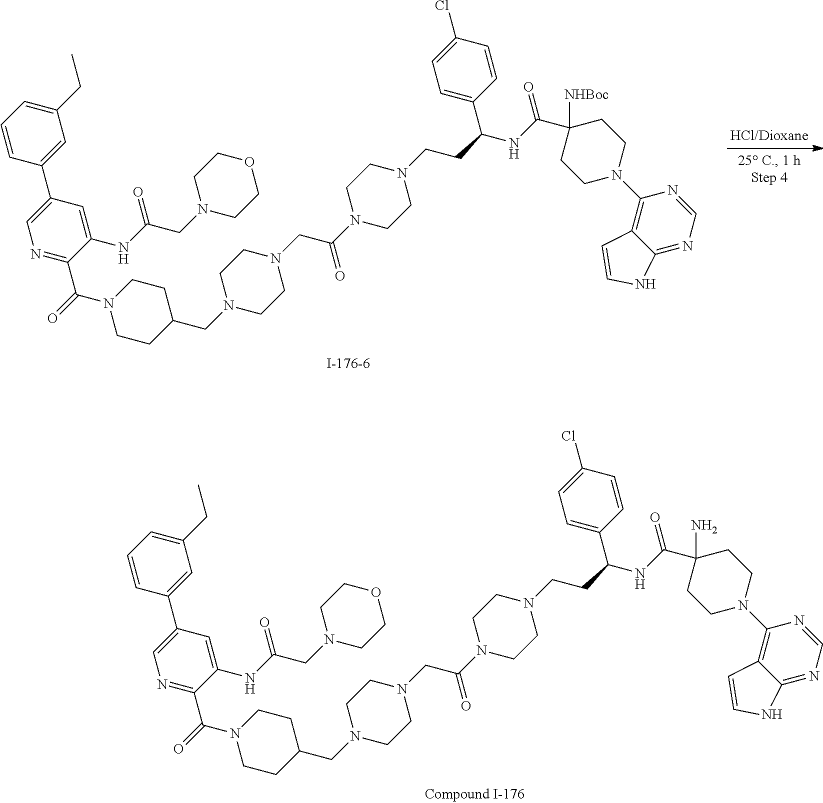 [SMILES: CCc1cccc(-c2cnc(C(=O)N3CCC(CN4CCN(CC(=O)N5CCN(CC[C@H](NC(=O)C6(NC(=O)OC(C)(C)C)CCN(c7ncnc8[nH]ccc78)CC6)c6ccc(Cl)cc6)CC5)CC4)CC3)c(NC(=O)CN3CCOCC3)c2)c1 ; CCc1cccc(-c2cnc(C(=O)N3CCC(CN4CCN(CC(=O)N5CCN(CC[C@H](NC(=O)C6(N)CCN(c7ncnc8[nH]ccc78)CC6)c6ccc(Cl)cc6)CC5)CC4)CC3)c(NC(=O)CN3CCOCC3)c2)c1]

### Step 1: Synthesis of I-176-3

To a solution of I-176-1 (200 mg, 541.40 μmol, 1 eq) in DMF (2 mL) was added HOAt (73.69 mg, 541.40 μmol, 75.74 μL, 1 eq), NMM (273.80 mg, 2.71 mmol, 297.61 μL, 5 eq) and EDCI (311.36 mg, 1.62 mmol, 3 eq). The mixture was stirred at 25° C. for 0.5 hr. Then I-176-2 (165.58 mg, 541.40 μmol, 1 eq, HCl salt) was added into the mixture and stirred at 25° C. for 0.5 hr. The reaction mixture was diluted with water (30 mL) and extracted with ethyl acetate (10 mL×3). The combined organic layers were washed with brine (10 mL×3) and dried over sodium sulfate, filtered and concentrated under reduced pressure to give a residue. The crude product was purified by reverse-phase HPLC (0.1% FA condition). The eluent was concentrated under reduced pressure to remove ACN and then lyophilized to give I-176-3 (300 mg, 483.26 μmol, 89.26% yield, 100% purity) as a brown solid. LCMS (Method D): R^{t}=0.297 min, (M+H)=621.5.

### Step 2: Synthesis of I-176-4

To a solution of I-176-3 (300 mg, 483.26 μmol, 1 eq) in THF (2 mL), MeOH (2 mL) and H₂O (1 mL) was added LiOH H₂O (60.84 mg, 1.45 mmol, 3 eq). The mixture was stirred at 40° C. for 1 hr. The mixture was concentrated to give a residue. The reaction mixture was diluted with water (60 mL). Then the mixture was adjusted to pH=6˜7 with saturated citric acid aqueous solution. Then the reaction mixture was extracted with DCM (20 mL×3). The combined organic layers were washed with brine (20 mL×3) and dried over sodium sulfate, filtered and concentrated under reduced pressure to give I-176-4 (100 mg, crude) was obtained as a brown solid. LCMS (Method D): R^{t}=0.266 min, (M+H)=593.3.

### Step 3: Synthesis of I-176-6

To a solution of I-176-4 (100 mg, 168.71 μmol, 1 eq) and I-176-5 (100.75 mg, 168.71 μmol, 1 eq) in DMF (1 mL) was added EDCI (97.03 mg, 506.14 μmol, 3 eq), NMM (85.32 mg, 843.56 μmol, 92.74 μL, 5 eq) and HOAt (22.96 mg, 168.71 μmol, 23.60 μL, 1 eq). The mixture was stirred at 25° C. for 1 hr. The reaction mixture was diluted with water (30 mL) and extracted with DCM (10 mL×3). The combined organic layers were washed with brine (10 mL×3) and dried over sodium sulfate, filtered and concentrated under reduced pressure to give a residue. The residue was purified by prep-HPLC (column: CD07-Daisogel SP-100-8-ODS-PK 150*25*10 um; mobile phase: [water (NH4HCO3)-ACN]; gradient: 42%-72% B over 12 min). The eluent was concentrated under reduced pressure to remove ACN and then lyophilized to give I-176-6 (100 mg, 82.85 μmol, 49.11% yield, 97.086% purity) as a brown solid. LCMS (Method D): R^{t}=0.329 min, (M+H)=1171.5. ¹H NMR (400 MHZ, METHANOL-d4) δ=8.88 (d, J=2.0 Hz, 1H), 8.60 (d, J=2.0 Hz, 1H), 8.12 (s, 1H), 7.56-7.47 (m, 2H), 7.43-7.41 (m, 1H), 7.32-7.28 (m, 5H), 7.12 (d, J=3.6 Hz, 1H), 6.61 (d, J=3.6 Hz, 1H), 5.49 (s, 2H), 5.02-4.99 (m, 1H), 4.89 (s, 1H), 4.73 (d, J=12.4 Hz, 1H), 4.37 (d, J=7.2 Hz, 2H), 3.89-3.76 (m, 5H), 3.70-3.55 (m, 6H), 3.20 (s, 3H), 3.18-3.10 (m, 1H), 2.98-2.85 (m, 1H), 2.74-2.72 (m, 2H), 2.65-2.59 (m, 4H), 2.59-2.26 (m, 14H), 2.23 (d, J=5.2 Hz, 2H), 2.20-2.04 (m, 5H), 2.00-1.85 (m, 3H), 1.81-1.69 (m, 1H), 1.51-1.39 (m, 9H), 1.31-1.27 (m, 3H). SFC: R^{t}=0.703 min.

### Step 4: Synthesis of I-176

To a solution of I-176-6 (100 mg, 85.33 μmol, 1 eq) in DCM (1 mL) was added HCl/dioxane (2 M, 200 μL). The mixture was stirred at 25° C. for 1 hr. The mixture was concentrated to give a residue. The crude product was purified by reverse-phase HPLC (0.1% HCl condition). The residue was concentrated under reduced pressure to remove ACN and then lyophilized to give I-176 (51.22 mg, 45.90 μmol, 53.79% yield, 99.317% purity, HCl salt) as a yellow solid. LCMS (Method D): R^{t}=0.282 min, (M+H)=1071.8. ¹H NMR (400 MHZ, METHANOL-d4) δ=8.75 (s, 1H), 8.40 (d, J=6.4 Hz, 2H), 7.61-7.49 (m, 2H), 7.44-7.42 (m, 4H), 7.35 (d, J=8.0 Hz, 3H), 7.02 (s, 1H), 5.07 (d, J=4.0 Hz, 1H), 4.73-4.49 (m, 5H), 4.39-4.25 (m, 2H), 4.08 (s, 2H), 3.95 (s, 8H), 3.86-3.77 (m, 2H), 3.66 (s, 12H), 3.60 (s, 2H), 3.39 (s, 5H), 3.28 (s. 2H), 3.10-2.96 (m, 1H), 2.84 (d, J=7.6 Hz. 1H), 2.75-2.72 (m, 2H), 2.53 (s. 1H), 2.43-2.19 (m, 3H). 2.10 (d, J=12.4 Hz, 2H), 1.98-1.86 (m, 1H), 1.74-1.58 (m, 1H), 1.54-1.51 (m, 1H), 1.29 (t. J=7.6 Hz. 3H). SFC: R^{t}=1.062 min.

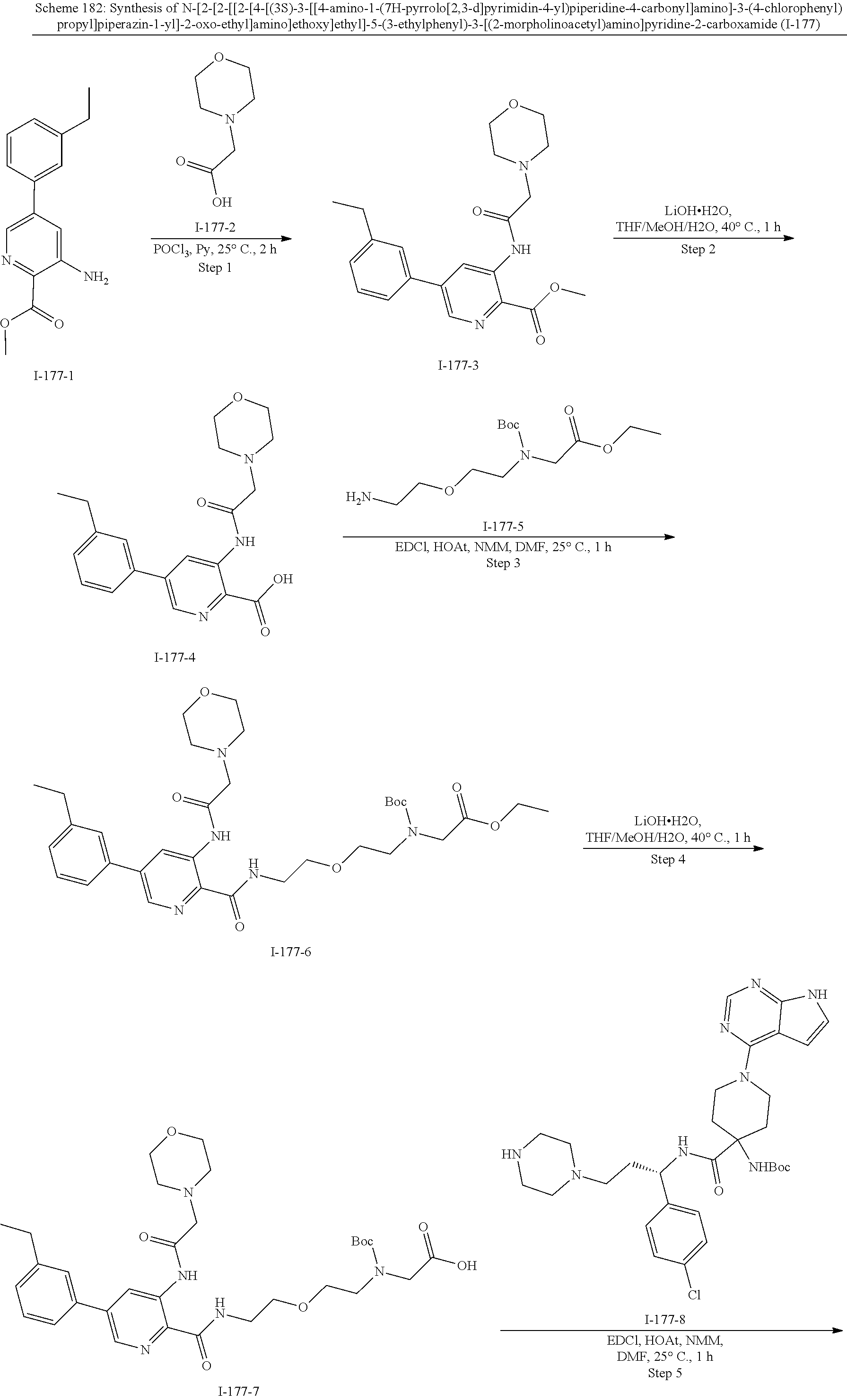

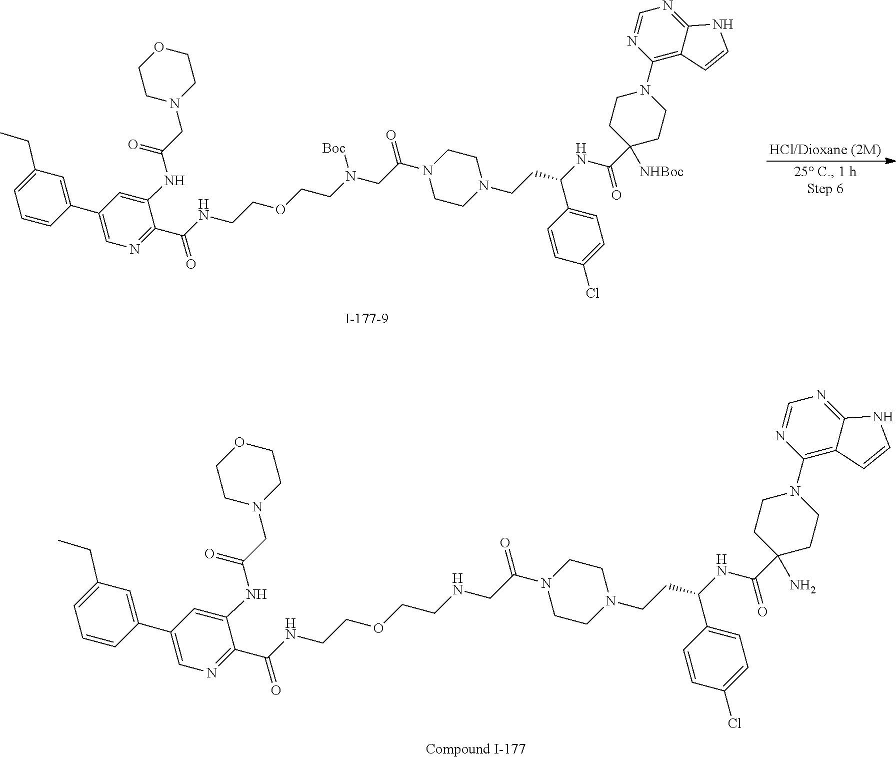 [SMILES: CCc1cccc(-c2cnc(C(=O)NCCOCCN(CC(=O)N3CCN(CC[C@H](NC(=O)C4(NC(=O)OC(C)(C)C)CCN(c5ncnc6[nH]ccc56)CC4)c4ccc(Cl)cc4)CC3)C(=O)OC(C)(C)C)c(NC(=O)CN3CCOCC3)c2)c1 ; CCc1cccc(-c2cnc(C(=O)NCCOCCNCC(=O)N3CCN(CC[C@H](NC(=O)C4(N)CCN(c5ncnc6[nH]ccc56)CC4)c4ccc(Cl)cc4)CC3)c(NC(=O)CN3CCOCC3)c2)c1]

### Step 1: Synthesis of I-177-3

To a solution of I-177-2 (500 mg, 3.44 mmol, 1 eq) and I-177-1 (1.06 g, 4.13 mmol, 1.2 eq) in Py (10 mL) was added POCl₃ (633.80 mg, 4.13 mmol, 385.29 μL, 1.2 eq). The mixture was stirred at 25° C. for 1 hr. The reaction mixture was diluted with water (60 mL) and extracted with ethyl acetate (20 mL×3). The combined organic layers were washed with brine (20 mL×3) and dried over sodium sulfate, filtered and concentrated under reduced pressure to give a residue. The residue was purified by flash silica gel chromatography (ISCO®; 20 g SepaFlash® Silica Flash Column, Eluent of 20˜70% Ethylacetate/Petroleum ether gradient @ 50 mL/min). The eluent was concentrated under reduced pressure to give I-177-3 (500 mg, 1.27 mmol, 36.99% yield, 97.7% purity) as a purple solid. LCMS (Method D): R^{t}=0.339 min, (M+H)=384.1.2. ¹H NMR (400 MHZ, CHLOROFORM-d) δ=12.19 (s, 1H), 9.45 (d, J=2.0 Hz, 1H), 8.67 (d, J=2.0 Hz, 1H), 7.51-7.47 (m, 2H), 7.43-7.39 (m, 1H), 7.30 (d, J=7.6 Hz, 1H), 4.06 (s, 3H), 3.94-3.87 (m, 4H), 3.25 (s, 2H), 2.79-2.65 (m, 6H), 1.31-1.27 (m, 3H).

### Step 2: Synthesis of I-177-4

To a solution of I-177-3 (450 mg, 1.17 mmol, 1 eq) in THF (2.5 mL), H₂O (1 mL) and MeOH (2.5 mL) was added LiOH H₂O (98.50 mg, 2.35 mmol, 2 eq). The mixture was stirred at 40° C. for 1 hr. The mixture was concentrated to give a residue. The reaction mixture was diluted with water (60 mL). Then the mixture was adjusted to pH=6˜7 with saturated citric acid aqueous solution. Then the reaction mixture was extracted with DCM (20 mL×3). The combined organic layers were washed with brine (20 mL×3) and dried over sodium sulfate, filtered and concentrated under reduced pressure to give I-177-4 (400 mg, 1.08 mmol, 92.26% yield) as a yellow solid. LCMS (Method D): R^{t}=0.281 min, (M+H)=370.1.

### Step 3: Synthesis of I-177-6

To a solution of I-177-4 (200 mg, 541.40 μmol, 1 eq) in DMF (2 mL) was added HOAt (73.69 mg, 541.40 μmol, 75.74 μL, 1 eq), NMM (273.80 mg, 2.71 mmol, 297.61 μL, 5 eq) and EDCI (311.36 mg, 1.62 mmol, 3 eq). The mixture and stirred at 25° C. for 0.5 hr. Then I-177-5 (157.20 mg, 541.40 μmol, 1 eq) was added into mixture and stirred at 25° C. for 0.5 hr. The reaction mixture was diluted with water (30 mL) and extracted with ethyl acetate (10 mL×3). The combined organic layers were washed with brine (10 mL×3) and dried over sodium sulfate, filtered and concentrated under reduced pressure to give a residue. The crude product was purified by reverse-phase HPLC (0.1% FA condition). The eluent was concentrated under reduced pressure to remove ACN and then lyophilized to give I-177-6 (250 mg, 373.82 μmol, 69.05% yield, 95.96% purity) as a brown solid. LCMS: R^{t}=0.841 min, (M+H)=642.5.

### Step 4: Synthesis of I-177-7

To a solution of I-177-6 (250 mg, 389.56 μmol, 1 eq) in THF (2 mL), H₂O (1 mL) and MeOH (2 mL) was added LiOH H₂O (49.04 mg, 1.17 mmol, 3 eq). The mixture was stirred at 40° C. for 1 hr. The mixture was concentrated to give a residue. The reaction mixture was diluted with water (60 mL). Then the mixture was adjusted to pH=6˜7 with saturated citric acid aqueous solution. Then the reaction mixture was extracted with DCM (20 mL×3). The combined organic layers were washed with brine (20 mL×3) and dried over sodium sulfate, filtered and concentrated under reduced pressure to give I-177-7 (220 mg, crude) as a brown oil. LCMS (Method D): R^{t}=0.399 min, (M+H)=614.3.

### Step 5: Synthesis of I-177-9

To a solution of I-177-7 (100 mg, 162.95 μmol, 1 eq) and I-177-8 (103.24 mg, 162.95 μmol, 1 eq, HCl salt) in DCM (2 mL) was added HOAt (22.18 mg, 162.95 μmol, 22.79 μL, 1 eq), EDCI (93.71 mg, 488.84 μmol, 3 eq) and NMM (82.41 mg, 814.73 μmol, 89.57 μL, 5 eq). The mixture was stirred at 25° C. for 1 hr. The reaction mixture was diluted with water (30 mL) and extracted with ethyl acetate (10 mL×3).

The combined organic layers were washed with brine (10 mL×3) and dried over sodium sulfate, filtered and concentrated under reduced pressure to give a residue. The crude product was purified by reverse-phase HPLC (0.1% FA condition). The eluent was concentrated under reduced pressure to remove ACN and then lyophilized to give I-177-9 (100 mg, 82.33 μmol, 50.53% yield, 98.206% purity) as a brown solid. LCMS (Method D): R^{t}=0.382 min, (M+H)=1192.6. ¹H NMR (400 MHZ, METHANOL-d4) δ=9.37 (d, J=2.0 Hz, 1H), 8.58 (d, J=7.6 Hz, 1H), 8.12 (s, 1H), 7.55-7.44 (m, 2H), 7.39-7.36 (m, 1H), 7.32-7.18 (m, 5H), 7.11 (d, J=3.6 Hz, 1H), 6.59 (d, J=3.6 Hz, 1H), 4.96 (d, J=3.6 Hz, 1H), 4.35 (d, J=11.2 Hz, 2H), 4.16 (d, J=10.4 Hz, 2H), 3.91-3.80 (m, 4H), 3.71-3.54 (m, 10H), 3.49-3.47 (m, 2H), 3.34 (s, 2H), 3.22 (s, 2H), 2.70-2.69 (m, 2H), 2.61 (s, 4H), 2.45-2.20 (m, 6H), 2.17-2.01 (m, 4H), 1.96-1.80 (m, 2H), 1.45 (d, J=12.4 Hz, 12H), 1.37 (s, 6H), 1.26 (t, J=7.6 Hz, 3H). SFC: R^{t}=0.506 min.

### Step 6: Synthesis of I-177

To a solution of I-177-9 (100 mg, 83.83 μmol, 1 eq) in DCM (1 mL) was added HCl/dioxane (2 M, 1 mL). The mixture was stirred at 25° C. for 1 hr. The mixture was concentrated to give a residue. The crude product was purified by reverse-phase HPLC (H2O). The eluent was concentrated under reduced pressure to remove ACN and then lyophilized to give I-177 (63.46 mg, 63.93 μmol, 76.26% yield, 100% purity) as a yellow solid. LCMS (Method D): R^{t}=0.302 min, (M+H)=992.5. ¹H NMR (400 MHZ, METHANOL-d4) δ=9.22 (s, 1H), 8.66 (s, 1H), 8.38 (s, 1H), 7.56-7.49 (m, 2H), 7.48-7.37 (m, 4H), 7.33 (d, J=4.8 Hz, 3H), 7.01 (s, 1H), 5.06 (s, 1H), 4.64-4.41 (m, 5H), 4.39-4.28 (m, 1H), 4.25-4.14 (m, 1H), 4.13-3.86 (m, 9H), 3.82-3.53 (m, 10H), 3.35 (s, 6H), 3.26-3.16 (m, 2H), 2.92-2.80 (m, 1H), 2.74-2.70 (m, 3H), 2.54 (s, 1H), 2.42-2.22 (m, 2H), 2.10 (d, J=13.2 Hz, 1H), 1.28-1.26 (m, 3H). SFC: R^{t}=0.848 min.

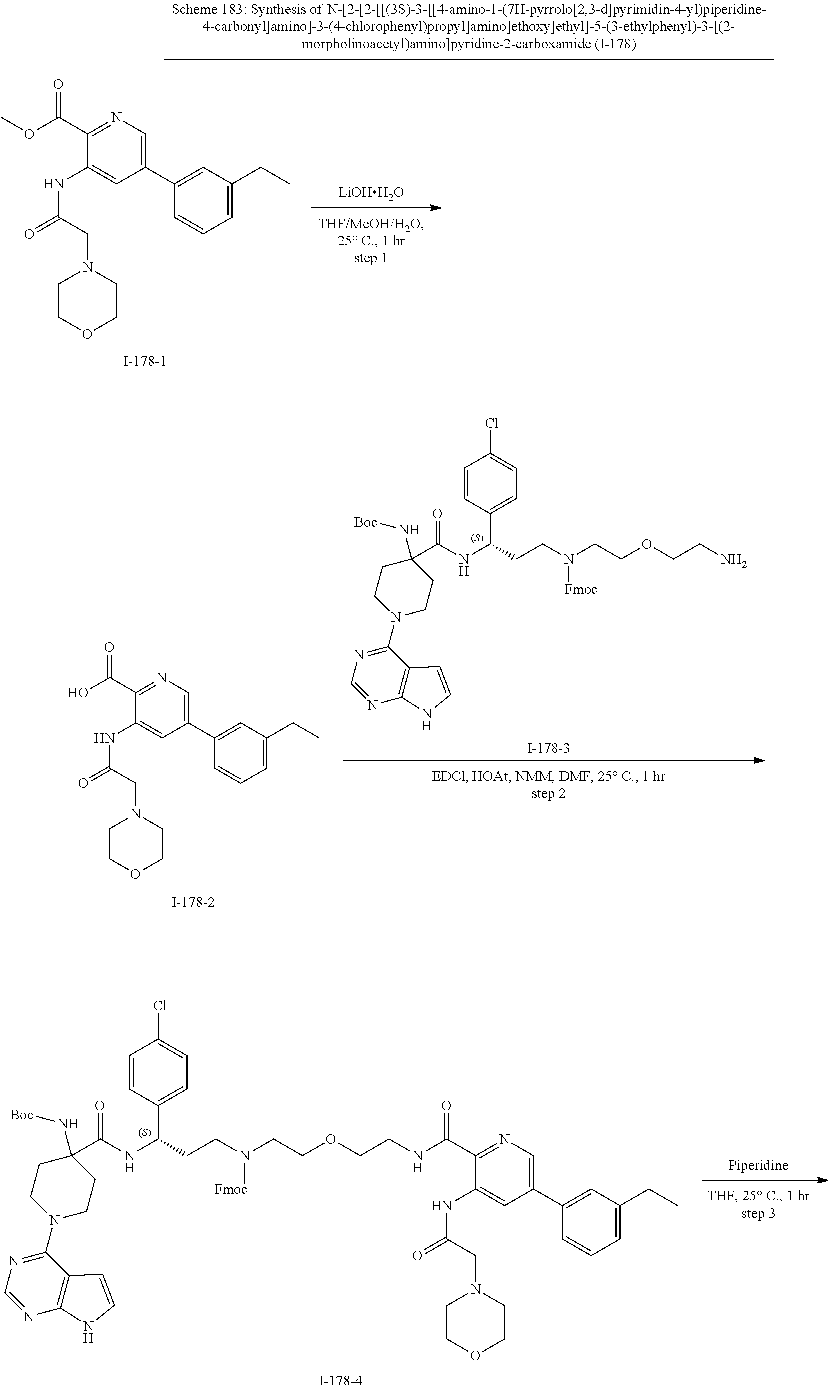

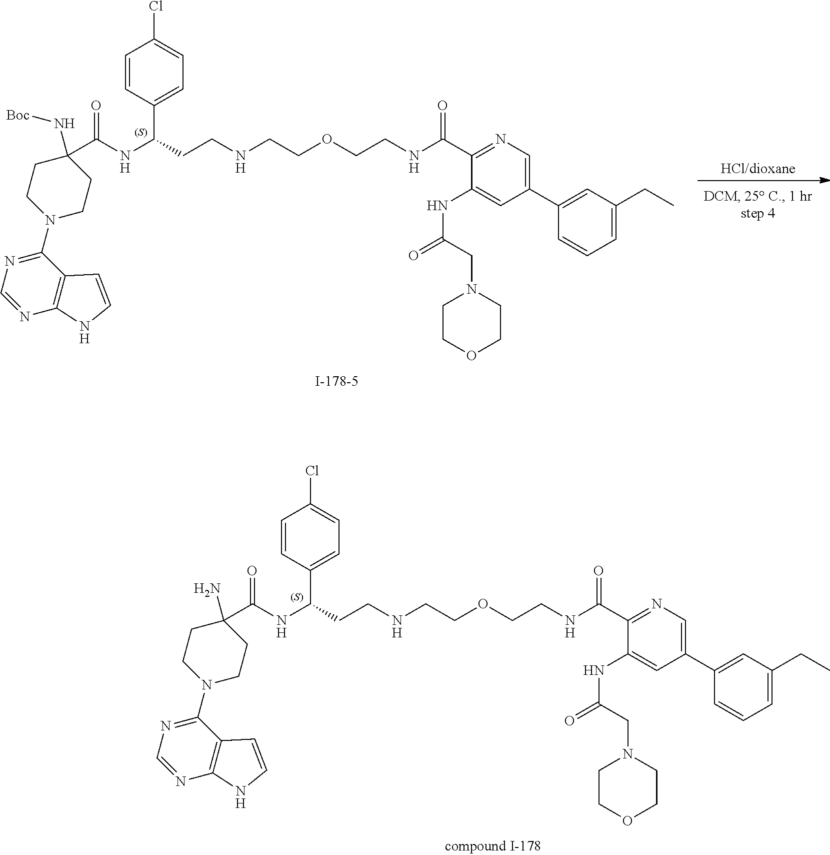

### Step 1: Synthesis of I-178-2

To a solution of I-178-1 (180 mg, 469.43 μmol, 1 eq) in THF (0.6 mL), MeOH (0.6 mL) and H₂O (0.6 mL) was added LiOH·H₂O (59.10 mg, 1.41 mmol, 3 eq). The mixture was stirred at 25° C. for 1 hr. The pH was acidified to 5˜6 with 1N HCl, and the mixture was washed with water (2 mL) and extract with EA (3 mL*3). The combined organic layers dried over sodium sulfate, filtered and concentrated under reduced pressure to give a residue. The crude product was used into the next step without further purification. I-178-2 (140 mg, 360.03 μmol, 76.69% yield, 95% purity) was obtained as a yellow solid. LCMS (Method D): R^{t}=0.290 min, (M+H)=370.2. ¹H NMR (400 MHZ, METHANOL-d4) δ=9.50 (s, 1H), 8.61 (s, 1H), 7.57-7.51 (m, 2H), 7.45-7.44 (m, 1H), 7.35 (d, J=7.2 Hz, 1H), 3.90-3.84 (m, 4H), 3.32 (s, 2H), 2.78-2.73 (m, 2H), 2.73-2.68 (m, 4H), 1.29-1.27 (m, 3H).

### Step 2: Synthesis of I-178-4

To a solution of I-178-2 (88.23 mg, 238.83 μmol, 1 eq) in DMF (2 mL) was added EDCI (137.35 mg, 716.50 μmol, 3 eq), HOAt (32.51 mg, 238.83 μmol, 33.41 μL, 1 eq) and NMM (120.79 mg, 1.19 mmol, 131.29 μL, 5 eq). Then I-178-3 (200 mg, 238.83 μmol, 1 eq) was added. The mixture was stirred at 25° C. for 1 hr. The mixture was washed with water (3 mL) and extract with EA (5 mL*3). The combined organic layers dried over sodium sulfate, filtered and concentrated under reduced pressure to give a residue. The residue was purified by flash silica gel chromatography (ISCO®; 80 g SepaFlash® Silica Flash Column, Eluent of 0˜100% MeOH/EA (100 mL/min) and then the eluent was concentrated in vacuo to give product. I-178-4 (130 mg, 108.26 μmol, 45.33% yield, 99% purity) was obtained as a white solid. LCMS (Method D): R^{t}=0.469 min, (M/2+H)=595.3. SFC: R^{t}=2.241 min. ¹H NMR (400 MHZ, METHANOL-d4) δ=9.26 (s, 1H), 8.43 (s, 1H), 8.16-7.96 (m, 2H), 7.85-7.69 (m, 2H), 7.55 (d, J=6.8 Hz, 2H), 7.46 (s, 2H), 7.42-7.39 (m, 1H), 7.28 (d, J=6.4 Hz, 4H), 7.24 (s, 2H), 7.19 (d, J=4.4 Hz, 2H), 7.10-7.03 (m, 1H), 6.53-6.44 (m, 1H), 4.50 (s, 2H), 4.36-4.25 (m, 2H), 4.24-4.09 (m, 2H), 3.88-3.83 (m, 4H), 3.62-3.48 (m, 7H), 3.41 (s, 1H), 3.25-3.12 (m, 7H), 2.72-2.70 (m, 2H), 2.58 (s, 4H), 2.21-2.10 (m, 2H), 2.06-2.01 (m, 2H), 1.81-1.68 (m, 1H), 1.38 (s, 9H), 1.28-1.25 (m, 3H).

### Step 3: Synthesis of I-178-5

To a solution of I-178-4 (100 mg, 84.12 μmol, 1 eq) in THF (1 mL) was added piperidine (107.78 mg, 1.27 mmol, 0.125 mL, 15.05 eq). The mixture was stirred at 25° C. for 1 hr. The reaction mixture was concentrated in vacuum. The crude product was used into the next step without further purification. I-178-5 (100 mg, crude) was obtained as yellow oil. LCMS (Method D): R^{t}=0.365 min, (M/2+H)=966.4.

### Step 4: Synthesis of 1-178

To a solution of I-178-5 (100 mg, 103.46 μmol, 1 eq) in DCM (1 mL) was added HCl/dioxane (2 M, 1.00 mL). The mixture was stirred at 25° C. for 0.5 hr. The pH was acidified to 5˜6 with NH₃·H₂O and the mixture was concentrated in vacuo. The residue was purified by flash silica gel chromatography (ISCO®; 12 g SepaFlash® Silica Flash Column, Eluent of 0˜100% MeOH (0.2% NH3: H2O)/DCM @ 30 mL/min) and then the eluent was concentrated in vacuo to give residue. The residue was purified by prep-HPLC (column: CD01-Phenomenex luna C18 150*25*10 um; mobile phase: [water (FA)-ACN]; gradient: 10%-40% B over 10 min) and the eluent was concentrated under reduced pressure to remove ACN and lyophilized to give product. I-178 (28.62 mg, 30.98 μmol, 29.94% yield, 98.771% purity, FA salt) was obtained as a white solid. LCMS (Method D): R^{t}=0.317 min, (M+H)=866.4. SFC: R^{t}=0.814 min. ¹H NMR (400 MHz, METHANOL-d4) δ=9.32 (d, J=2.0 Hz, 1H), 8.46 (d, J=2.0 Hz, 1H), 8.39 (s, 2H), 8.11 (s, 1H), 7.45 (s, 1H), 7.41-7.38 (m, 2H), 7.33-7.28 (m, 5H), 7.10 (d, J=3.6 Hz, 1H), 6.56 (d, J=3.6 Hz, 1H), 4.99-4.97 (m, 1H), 4.48-4.42 (m, 1H), 4.40-4.34 (m, 1H), 3.87-3.84 (m, 4H), 3.78-3.77 (m, 2H), 3.73 (d, J=3.6 Hz, 2H), 3.71-3.68 (m, 1H), 3.65 (d, J=2.8 Hz, 1H), 3.63-3.56 (m, 2H), 3.25-3.24 (m, 2H), 3.21 (s, 2H), 3.17-3.12 (m, 1H), 3.00-2.99 (m, 1H), 2.72-2.71 (m, 2H), 2.62-2.58 (m, 4H), 2.32-2.25 (m, 1H), 2.23-2.17 (m, 3H), 1.66 (d, J=13.6 Hz, 1H), 1.57 (d, J=14.0 Hz, 1H), 1.28-1.25 (m, 3H).

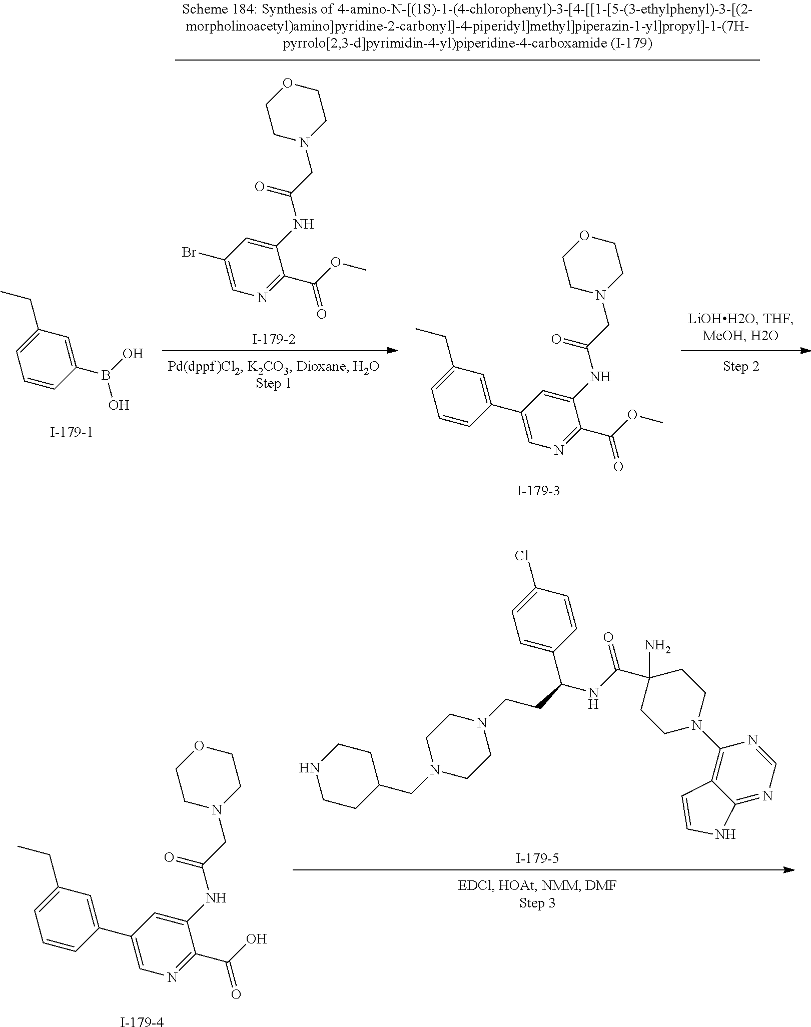

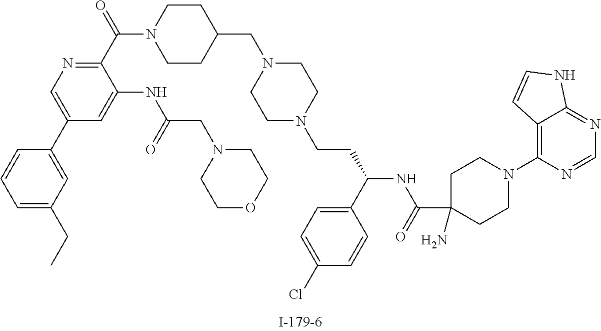 [SMILES: CCc1cccc(-c2cnc(C(=O)N3CCC(CN4CCN(CC[C@H](NC(=O)C5(N)CCN(c6ncnc7[nH]ccc67)CC5)c5ccc(Cl)cc5)CC4)CC3)c(NC(=O)CN3CCOCC3)c2)c1]

### Step 1: Synthesis of I-179-3

To a solution of I-179-1 (100 mg, 666.75 μmol, 1 eq) and I-179-2 (238.82 mg, 666.75 μmol, 1 eq) in dioxane (2 mL) and H₂O (0.5 mL) was added Pd(dppf)Cl₂ (48.79 mg, 66.67 μmol, 0.1 eq) and K₂CO₃ (276.44 mg, 2.00 mmol, 3 eq). The suspension was degassed and purged with N₂ for 3 times. The mixture was stirred at 90° C. for 1 hr. The reaction mixture was diluted with water (30 mL) and extracted with ethyl acetate (10 mL×3). The combined organic layers were washed with brine (10 mL×3) and dried over sodium sulfate, filtered and concentrated under reduced pressure to give a residue. The crude product was purified by reverse-phase HPLC (0.1% FA condition). The eluent was concentrated under reduced pressure to remove ACN and then lyophilized to give I-179-3 (200 mg, 521.59 μmol, 78.23% yield) as a brown solid. LCMS (Method D): R^{t}=0.338 min, (M+H)=384.1.

### Step 2: Synthesis of I-179-4

To a solution of I-179-3 (200 mg, 521.59 μmol, 1 eq) in THF (2 mL), MeOH (2 mL) and H₂O (1 mL) was added LiOH H₂O (65.66 mg, 1.56 mmol, 3 eq). The mixture was stirred at 40° C. for 1 hr. The mixture was concentrated to give I-179-4 (100 mg, crude) as a brown solid. LCMS (Method D): R^{t}=0.286 min, (M+H)=370.1.

### Step 3: Synthesis of I-179

To a solution of I-179-4 (70 mg, 189.49 μmol, 1 eq) in DMF (2 mL) was added EDCI (108.98 mg, 568.47 μmol, 3 eq), NMM (95.83 mg, 947.45 μmol, 104.16 μL, 5 eq) and HOAt (25.79 mg, 189.49 μmol, 26.51 μL, 1 eq). The mixture was stirred at 25° C. for 0.5 hr. Then I-179-5 (119.50 mg, 189.49 μmol, 1 eq, HCl salt) was added into the mixture and stirred at 25° C. for 0.5 hr. The reaction mixture was diluted with water (30 mL) and extracted with DCM (10 mL×3). The combined organic layers were washed with brine (10 mL×3) and dried over sodium sulfate, filtered and concentrated under reduced pressure to give a residue. The crude product was purified by reverse-phase HPLC (H2O). The eluent was concentrated under reduced pressure to remove ACN and then lyophilized to give I-179 (23.8 mg, 23.84 μmol, 12.58% yield, 94.702% purity) as a yellow solid. LCMS: R^{t}=0.730 min, (M+H)=945.5. ¹H NMR (400 MHZ, DMSO-d6) δ=11.64 (s, 1H), 10.37 (s, 1H), 8.84 (d, J=2.0 Hz, 1H), 8.74-8.47 (m, 2H), 8.11 (s, 1H), 7.59-7.49 (m, 2H), 7.45-7.43 (m, 1H), 7.37-7.27 (m, 5H), 7.19-7.11 (m, 1H), 6.57 (d, J=1.6 Hz, 1H), 4.82-4.80 (m, 1H), 4.57 (d, J=12.4 Hz, 1H), 4.39-4.35 (m, 2H), 3.85-3.60 (m, 6H), 3.59-3.46 (m, 2H), 3.15 (s, 2H), 3.06-3.02 (m, 1H), 2.84-2.78 (m, 1H), 2.70-2.67 (m, 2H), 2.52 (s, 3H), 2.35 (s, 8H), 2.18 (s, 4H), 2.12-2.07 (m, 2H), 2.01-1.78 (m, 6H), 1.64 (d, J=12.4 Hz, 1H), 1.43-1.30 (m, 2H), 1.23-1.21 (m, 3H), 1.12-0.94 (m, 2H). SFC: R^{t}=0.696 min.

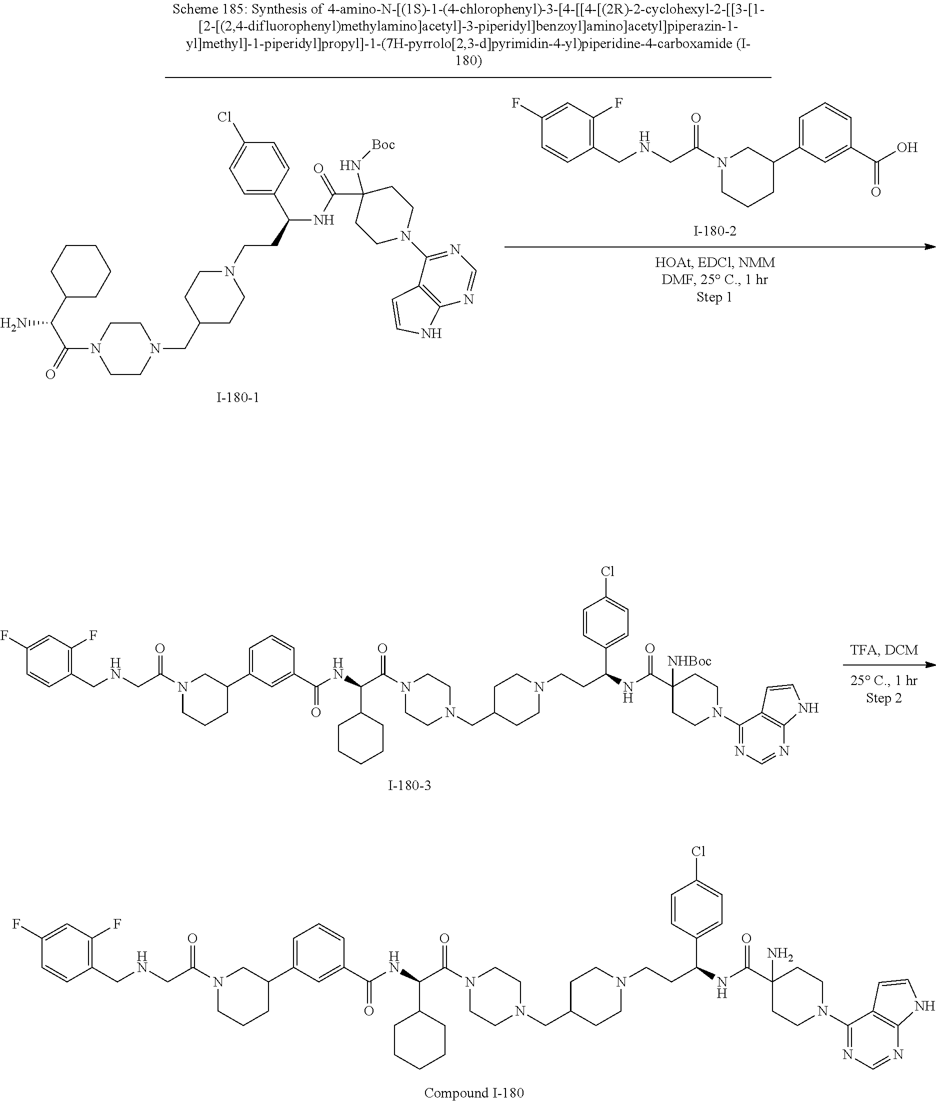

### Step 1: Synthesis of I-180-3

To a solution of I-180-2 (35.17 mg, 71.99 μmol, 1 eq) in DMF (1 mL) was added EDCI (41.40 mg, 215.96 μmol, 3 eq), HOAt (9.80 mg, 71.99 μmol, 10.07 μL, 1 eq), I-180-1 (60 mg, 71.99 μmol, 1 eq) and NMM (36.41 mg, 359.93 μmol, 39.57 μL, 5 eq). The mixture was stirred at 25° C. for 1 hr. The reaction mixture was diluted with H₂O 1.5 mL and extracted with EA 4 mL (2 mL*2). The combined organic layers were washed with brine 2 mL (2 mL*1), dried over Na₂SO₄, filtered, and concentrated under reduced pressure to give I-180-3 (100 mg, crude) as yellow oil. LCMS (Method D): R^{t}=0.416 min, (M+H)=1303.4.

### Step 2: Synthesis of I-180

To a solution of I-180-3 (90 mg, 69.02 μmol, 1 eq) in CH₂Cl₂ (1 mL) and TFA (0.2 mL) was stirred at 25° C. for 1 hr. The mixture was filtered. The filtered liquor was purified by reverse-phase HPLC (0.1% TFA condition). The eluent was concentrated under reduced pressure to remove ACN and then lyophilized to give the product. The residue was purified by prep-HPLC (column: CD01-Phenomenex luna C18 150*25*10 um; mobile phase: [water (TFA)-ACN]; gradient: 13%-43% B over 10 min). The eluent was concentrated under reduced pressure to remove ACN and then lyophilized to give I-180 (19.98 mg, 16.37 μmol, 23.72% yield, 99.8% purity, TFA salt) was obtained as a white solid. LCMS (Method D): R^{t}=0.293 min, (M+H)=1103.5. ¹H NMR (400 MHZ, METHANOL-d4) δ=8.37 (s, 1H), 7.80-7.69 (m, 2H), 7.66-7.56 (m, 1H), 7.55-7.42 (m, 2H), 7.40-7.31 (m, 5H), 7.19-7.05 (m, 2H), 6.91 (d, J=3.6 Hz, 1H), 5.05-4.98 (m, 1H), 4.85-4.78 (m, 2H), 4.67-4.50 (m, 3H), 4.34-4.29 (m, 2H), 4.26-4.17 (m, 2H), 4.16-4.06 (m, 1H), 3.92-3.82 (m, 2H), 3.77-3.69 (m, 1H), 3.61 (s, 2H), 3.51-3.34 (m, 3H), 3.28-3.18 (m, 3H), 3.16-3.06 (m, 3H), 3.06-2.94 (m, 2H), 2.92-2.73 (m, 2H), 2.72-2.55 (m, 2H), 2.41-1.53 (m, 21H), 1.38-1.20 (m, 3H), 1.19-1.04 (m, 2H) ¹⁹F NMR (376 MHz, METHANOL-d4) δ=−76.977, —108.425, −112.860. SFC: R^{t}=3.544 min, 4.580 min.

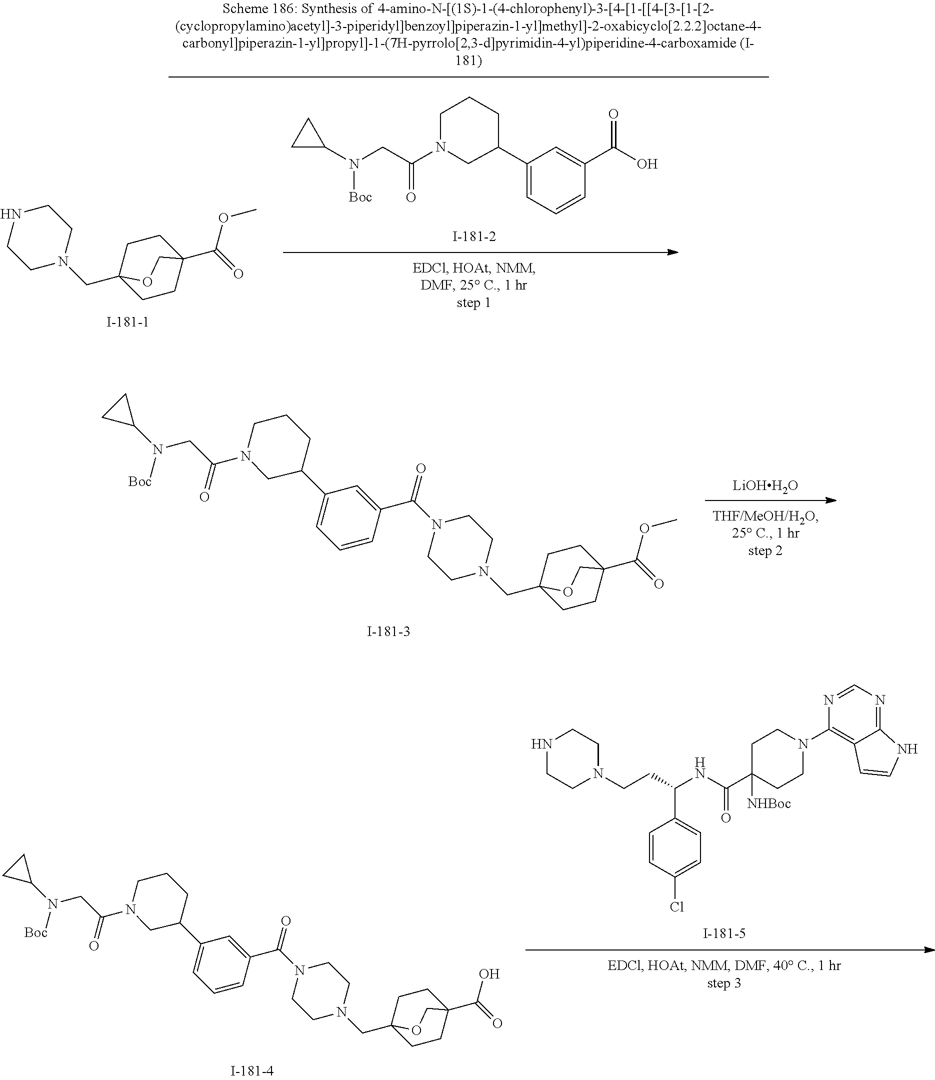 [SMILES: COC(=O)C12CCC(CN3CCNCC3)(CC1)OC2 ; CC(C)(C)OC(=O)N(CC(=O)N1CCCC(c2cccc(C(=O)O)c2)C1)C1CC1 ; COC(=O)C12CCC(CN3CCN(C(=O)c4cccc(C5CCCN(C(=O)CN(C(=O)OC(C)(C)C)C6CC6)C5)c4)CC3)(CC1)OC2 ; "LiOH·H2O" ; CC(C)(C)OC(=O)N(CC(=O)N1CCCC(c2cccc(C(=O)N3CCN(CC45CCC(C(=O)O)(CC4)CO5)CC3)c2)C1)C1CC1 ; CC(C)(C)OC(=O)NC1(C(=O)N[C@@H](CCN2CCNCC2)c2ccc(Cl)cc2)CCN(c2ncnc3[nH]ccc23)CC1]

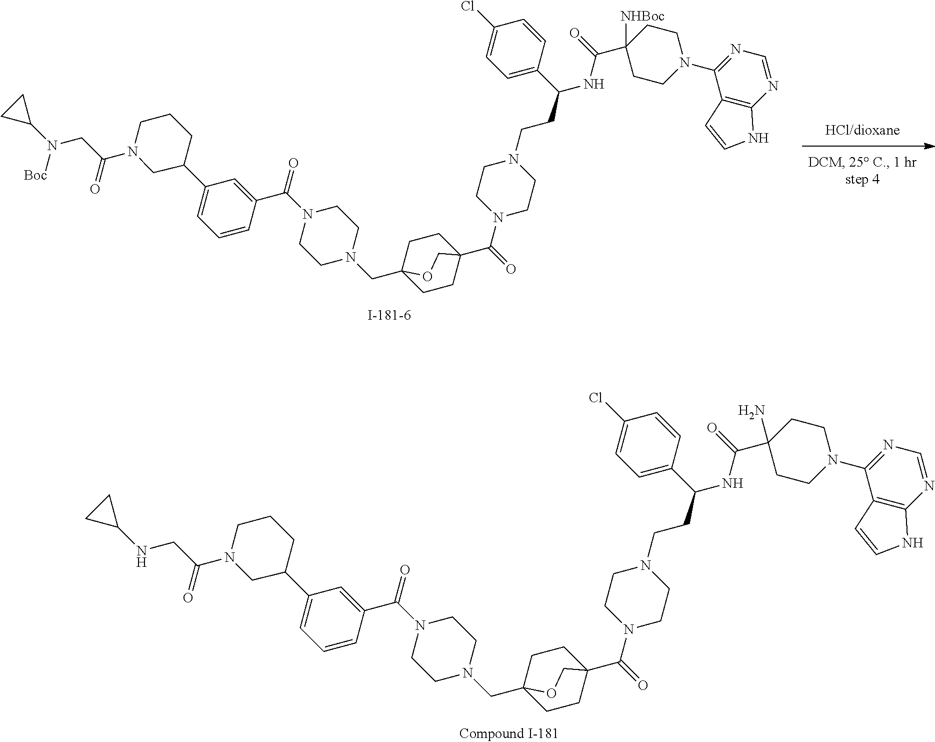 [SMILES: CC(C)(C)OC(=O)NC1(C(=O)N[C@@H](CCN2CCN(C(=O)C34CCC(CN5CCN(C(=O)c6cccc(C7CCCN(C(=O)CN(C(=O)OC(C)(C)C)C8CC8)C7)c6)CC5)(CC3)OC4)CC2)c2ccc(Cl)cc2)CCN(c2ncnc3[nH]ccc23)CC1 ; NC1(C(=O)N[C@@H](CCN2CCN(C(=O)C34CCC(CN5CCN(C(=O)c6cccc(C7CCCN(C(=O)CNC8CC8)C7)c6)CC5)(CC3)OC4)CC2)c2ccc(Cl)cc2)CCN(c2ncnc3[nH]ccc23)CC1]

### Step 1: Synthesis of I-181-3.

To a solution of I-181-1 (200 mg, 656.14 μmol, 1 eq, HCl) in DMF (5 mL) was added I-181-2 (299.97 mg, 745.30 μmol, 1.14 eq), EDCI (377.35 mg, 1.97 mmol, 3 eq), HOAt (89.31 mg, 656.14 μmol, 91.79 μL, 1 eq) and NMM (331.83 mg, 3.28 mmol, 360.69 μL, 5 eq). The mixture was stirred at 25° C. for 1 hr. The reaction mixture was diluted with H₂O 5 mL and extracted with EA 10 mL (5 mL*2). The combined organic layers were washed with brine 2 mL (2 mL*1), dried over Na₂SO₄, filtered, and concentrated under reduced pressure to give a residue. The residue was purified by flash silica gel chromatography (ISCO®; 20 g SepaFlash® Silica Flash Column, Eluent of 0˜100% Ethylacetate/Petroleum ether gradient @; 40 mL/min). The eluent was concentrated under reduced pressure to give I-181-3 (300 mg, 447.23 μmol, 68.16% yield, 97.319% purity) as yellow oil. LCMS (Method D): R^{t}=0.361 min, [M+H]⁺=653.4. SFC: R^{t}=1.568 min, 2.001 min. ¹H NMR (400 MHZ, METHANOL-d4) δ=7.46-7.38 (m, 2H), 7.37-7.23 (m, 2H), 5.49 (s, 2H), 4.60-4.49 (m, 1H), 4.23-4.15 (m, 1H), 4.13-4.04 (m, 1H), 3.90 (s, 3H), 3.75-3.72 (m, 1H), 3.64 (s, 2H), 3.50-3.35 (m, 2H), 3.31 (s, 2H), 3.25-3.09 (m, 1H), 2.76-2.68 (m, 2H), 2.64-2.61 (m, 1H), 2.57-2.46 (m, 2H), 2.31 (s, 2H), 2.09-2.02 (m, 1H), 2.00-1.90 (m, 6H), 1.88-1.83 (m, 1H), 1.71-1.57 (m, 3H), 1.46 (s, 9H), 1.37-1.18 (m, 1H), 0.76-0.59 (m, 4H).

### Step 2: Synthesis of I-181-4.

To a solution of I-181-3 (280 mg, 428.91 μmol, 1 eq) in MeOH (2 mL), THF (2 mL) and H₂O (2 mL) was added LiOH H₂O (54.00 mg, 1.29 mmol, 3 eq). The mixture was stirred at 25° C. for 1 hr. The mixture was diluted with H₂O (1 mL) and added HCl (1M) to adjust to a pH=4. The mixture was extracted with EA 2 mL (1 mL*2). The combined organic layers were dried over Na₂SO₄, filtered, and concentrated under reduced pressure to give a residue. The crude product was purified by reverse-phase HPLC (neutral condition). The eluent was concentrated under reduced pressure to give I-181-4 (200 mg, 305.62 μmol, 71.26% yield, 97.614% purity) as yellow oil. LCMS (Method D): R^{t}=0.343 min, [M+H]⁺=639.3. SFC: R^{t}=0.977 min, 1.139 min. ¹H NMR (400 MHZ, METHANOL-d4) δ=7.49-7.35 (m, 4H), 4.57-4.48 (m, 1H), 4.23-4.15 (m, 1H), 4.00 (s, 3H), 3.96-3.80 (m, 4H), 3.49-3.36 (m, 4H), 3.23-3.14 (m, 3H), 2.88-2.64 (m, 4H), 2.07-2.02 (m, 6H), 1.89-1.75 (m, 4H), 1.46 (s, 9H), 1.15 (d, J=6.0 Hz, 2H), 0.73-0.63 (m, 4H).

### Step 3: Synthesis of I-181-6.

To a solution of I-181-4 (64.18 mg, 100.48 μmol, 1 eq) in DMF (1 mL) was added I-181-5 (60 mg, 100.48 umol, 1 eq), EDCI (57.78 mg, 301.43 μmol, 3 eq), HOAt (13.68 mg, 100.48 μmol, 14.06 μL, 1 eq) and NMM (50.81 mg, 502.39 μmol, 55.23 μL, 5 eq). The mixture was stirred at 40° C. for 1 hr. The reaction mixture was diluted with H₂O 1 mL and extracted with EA 3 ml (1.5 mL*2). The combined organic layers were washed with brine 2 mL (2 mL*1), dried over Na₂SO₄, filtered, and concentrated under reduced pressure to give a residue. The residue was purified by flash silica gel chromatography (ISCO®; 20 g SepaFlash® Silica Flash Column, Eluent of 0˜10% MeOH/DCM @ 60 mL/min). The eluent was concentrated under reduced pressure to give I-181-6 (110 mg, 84.18 μmol, 83.78% yield, 93.2% purity) as yellow oil.

### Step 4: Synthesis of I-181

To a solution of I-181-6 (90 mg, 73.90 μmol, 1 eq) in DCM (1 mL) and HCl/dioxane (2 M, 9.00 mL) was stirred at 25° C. for 1 hr. LC-MS( ). The mixture was concentrated in vacuum. The crude product was purified by prep-HPLC (column: Phenomenex luna C18 150*25 mm*10 um; mobile phase: [water (HCl)-ACN]; gradient: 0%-25% B over 14 min). The eluent was concentrated under reduced pressure to remove ACN and then lyophilized to give I-181 (18.61 mg, 16.85 μmol, 22.81% yield, 95.465% purity, HCl) was obtained as a white solid. LCMS: R^{t}=0.241 min, [M+H]⁺=1017.4. SFC: R^{t}=2.499 min, 2.923 min. ¹H NMR (400 MHZ, METHANOL-d4) δ=8.39 (s, 1H), 7.56-7.49 (m, 1H), 7.48-7.37 (m, 6H), 7.35 (d, J=8.4 Hz, 2H), 7.02 (d, J=3.2 Hz, 1H), 5.09-5.01 (m, 1H), 4.71-4.47 (m, 6H), 4.44-4.12 (m, 3H), 4.11-3.90 (m, 5H), 3.86-3.56 (m, 8H), 3.41-3.33 (m, 3H), 3.29-3.18 (m, 3H), 3.18-3.06 (m, 2H), 2.96-2.71 (m, 5H), 2.61-2.46 (m, 1H), 2.37-2.21 (m, 4H), 2.18-1.93 (m, 7H), 1.92-1.77 (m, 4H), 1.75-1.54 (m, 1H), 0.99-0.87 (m, 4H).

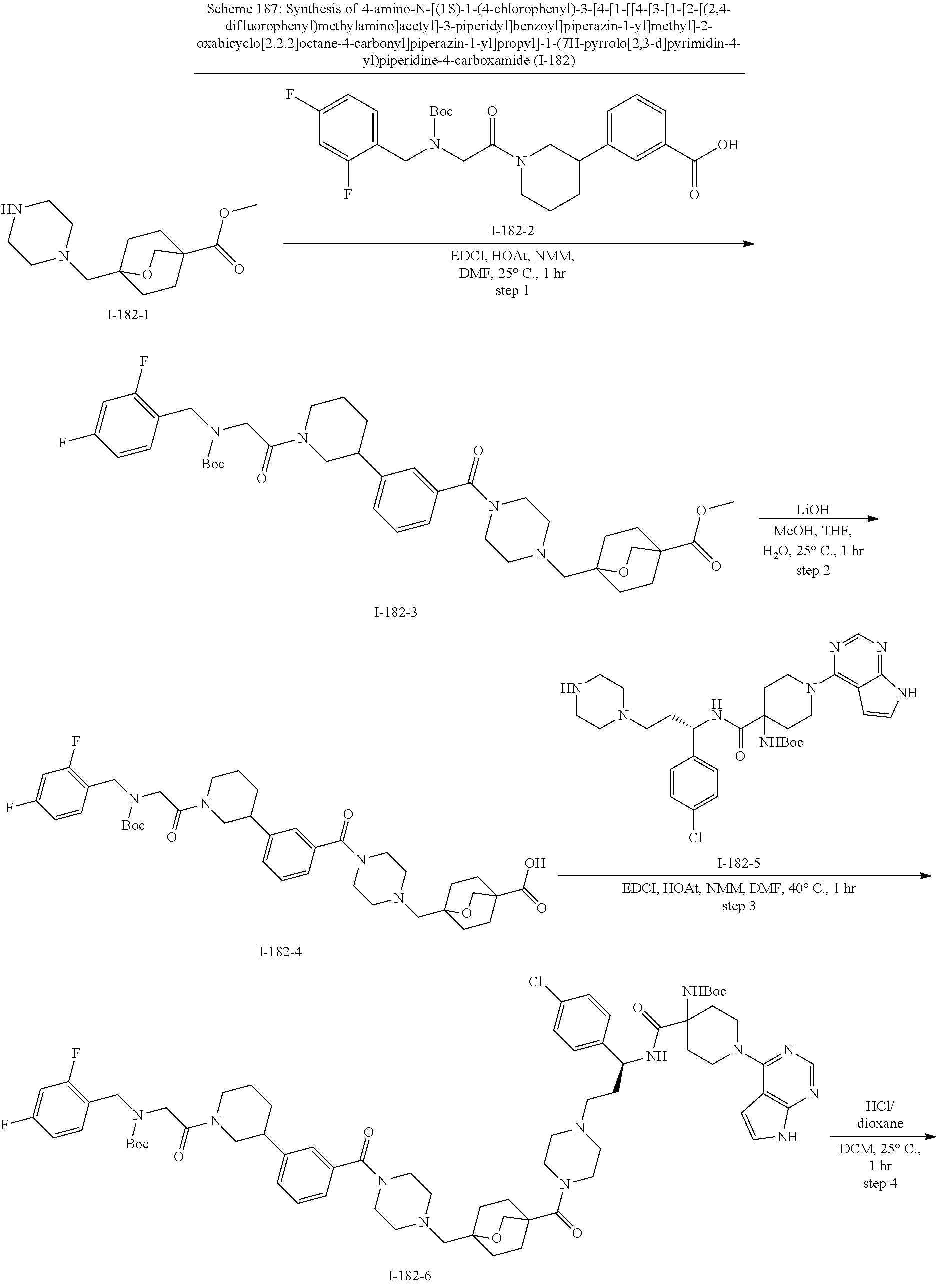

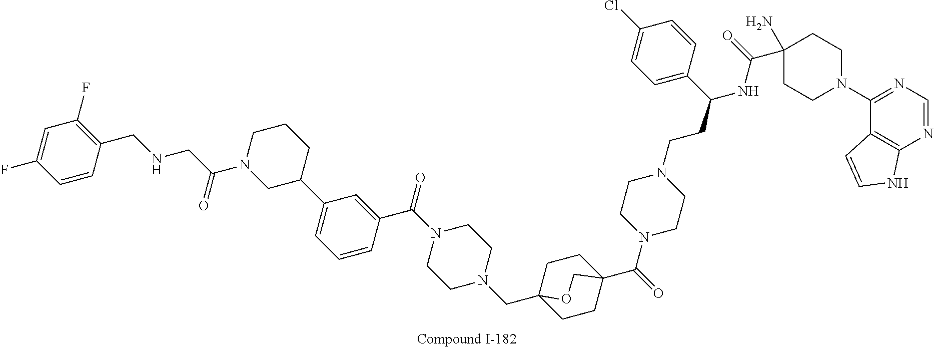 [SMILES: NC1(C(=O)N[C@@H](CCN2CCN(C(=O)C34CCC(CN5CCN(C(=O)c6cccc(C7CCCN(C(=O)CNCc8ccc(F)cc8F)C7)c6)CC5)(CC3)OC4)CC2)c2ccc(Cl)cc2)CCN(c2ncnc3[nH]ccc23)CC1]

### Step 1: Synthesis of I-182-3.

To a solution of I-182-1 (200 mg, 656.14 μmol, 1 eq, HCl) in DMF (5 mL) was added I-182-2 (364.09 mg, 745.29 μmol, 1.14 eq), EDCI (377.35 mg, 1.97 mmol, 3 eq), HOAt (89.31 mg, 656.14 μmol, 91.79 μL, 1 eq) and NMM (331.83 mg, 3.28 mmol, 360.69 μL, 5 eq). The mixture was stirred at 25° C. for 1 hr. The reaction mixture was diluted with H₂O 5 mL and extracted with EA 10 mL (5 mL*2). The combined organic layers were washed with brine 2 mL (2 mL*1), dried over Na₂SO₄, filtered, and concentrated under reduced pressure to give a residue. The residue was purified by flash silica gel chromatography (ISCO®; 20 g SepaFlash® Silica Flash Column, Eluent of 0˜100% Ethylacetate/Petroleum ether gradient @ 60 mL/min). The eluent was concentrated under reduced pressure to give I-182-3 (270 mg, 338.78 μmol, 51.63% yield, 92.708% purity) was obtained as yellow oil. LCMS (Method D): R^{t}=0.434 min, [M+H]⁺=739.3. SFC: R^{t}=1.215 min, 1.739 min. ¹H NMR (400 MHZ, METHANOL-d4) δ=7.45-7.34 (m, 3H), 7.33-7.24 (m, 2H), 7.01-6.91 (m, 2H), 4.59-4.42 (m, 3H), 4.28-4.15 (m, 1H), 3.90 (s, 2H), 3.79-3.68 (m, 2H), 3.64 (s, 3H), 3.43-3.39 (m, 1H), 3.20-3.08 (m, 1H), 2.81-2.67 (m, 2H), 2.66-2.58 (m, 2H), 2.55-2.45 (m, 2H), 2.31 (s, 2H), 2.08-2.02 (m, 1H), 2.00-1.91 (m, 6H), 1.88-1.76 (m, 2H), 1.66-1.61 (m, 2H), 1.47-1.41 (m, 9H), 1.32-1.28 (m, 2H), 0.97-0.79 (m, 2H), 19FNMR (376 MHz, MeOD-d6) δ=−113.454, −115.683.

### Step 2: Synthesis of I-182-4.

To a solution of I-182-3 (250 mg, 338.36 μmol, 1 eq) in MeOH (2 mL), THF (2 mL) and H₂O (2 mL) was added LiOH H₂O (42.60 mg, 1.02 mmol, 3 eq). The mixture was stirred at 25° C. for 1 hr. The mixture was diluted with H₂O (1 mL) and added HCl (1M) to adjust to a pH=4. The mixture was extracted with EA 2 mL (1 mL*2). The combined organic layers were dried over Na₂SO₄, filtered, and concentrated under reduced pressure to give a residue. The crude product was purified by reverse-phase HPLC (neutral condition). The eluent was concentrated under reduced pressure to give I-182-4 (180 mg, 243.29 μmol, 71.90% yield, 97.971% purity) was obtained as yellow oil. LCMS (Method D): R^{t}=0.394 min, [M+H]⁺=725.3. SFC: R^{t}=2.197 min, 2.339 min. ¹H NMR (400 MHZ, METHANOL-d4) δ=7.54-7.29 (m, 5H), 7.05-6.85 (m, 2H), 4.60-4.40 (m, 3H), 4.28-4.04 (m, 3H), 3.91-3.81 (m, 2H), 3.46-3.40 (m, 2H), 3.26-3.08 (m, 3H), 2.85-2.65 (m, 2H), 2.06-2.01 (m, 6H), 1.90-1.75 (m, 4H), 1.70-1.52 (m, 2H), 1.48-1.40 (m, 9H), 1.39-1.27 (m, 4H), 0.98-0.82 (m, 2H), 19FNMR (376 MHz, MeOD-d6) δ=−113.375, −115.792.

### Step 3: Synthesis of I-182-6.

To a solution of I-182-4 (72.83 mg, 100.48 μmol, 1 eq) in DMF (1 mL) was added I-182-5 (60 mg, 100.48 μmol, 1 eq), EDCI (57.78 mg, 301.43 μmol, 3 eq), HOAt (13.68 mg, 100.48 μmol, 14.06 μL, 1 eq) and NMM (50.81 mg, 502.39 μmol, 55.23 μL, 5 eq). The mixture was stirred at 40° C. for 1 hr. The reaction mixture was diluted with H₂O 1 mL and extracted with EA 3 mL (1.5 mL*2). The combined organic layers were washed with brine 2 mL (2 mL*1), dried over Na₂SO₄, filtered, and concentrated under reduced pressure to give a residue. The residue was purified by flash silica gel chromatography (ISCO®; 20 g SepaFlash® Silica Flash Column, Eluent of 0˜10% MeOH/DCM (@ 60 mL/min). The eluent was concentrated under reduced pressure to give I-182-6 (100 mg, 76.69 μmol, 76.32% yield) was obtained as a white solid. LCMS (Method D): R^{t}=0.376 min, [M+H]⁺=1303.5. SFC: R^{t}=1.321 min, 1.710 min.

### Step 4: Synthesis of I-182

To a solution of I-182-6 (90 mg, 69.02 μmol, 1 eq) in DCM (1 mL) and HCl/dioxane (2 M, 9.00 mL) was stirred at 25° C. for 1 hr. LC-MS( ). The mixture was concentrated in vacuum. The crude product was purified by prep-HPLC (column: Phenomenex luna C18 150*25 mm*10 um; mobile phase: [water (HCl)-ACN]; gradient: 1%-31% B over 14 min). The eluent was concentrated under reduced pressure to remove ACN and then lyophilized to give I-182 (25.95 mg, 22.45 μmol, 32.53% yield, 98.639% purity, HCl) was obtained as a white solid. LCMS: R^{t}=0.265 min, [M+H]⁺=1103.4. SFC: R^{t}=3.429 min, 3.864 min. ¹H NMR (400 MHZ, METHANOL-d4) δ=8.39 (s, 1H), 7.71-7.62 (m, 1H), 7.53 s4H), 1.75-1.58 (m, 1H), 19FNMR (376 MHz, MeOD-d6) δ=−108.463, −112.808.

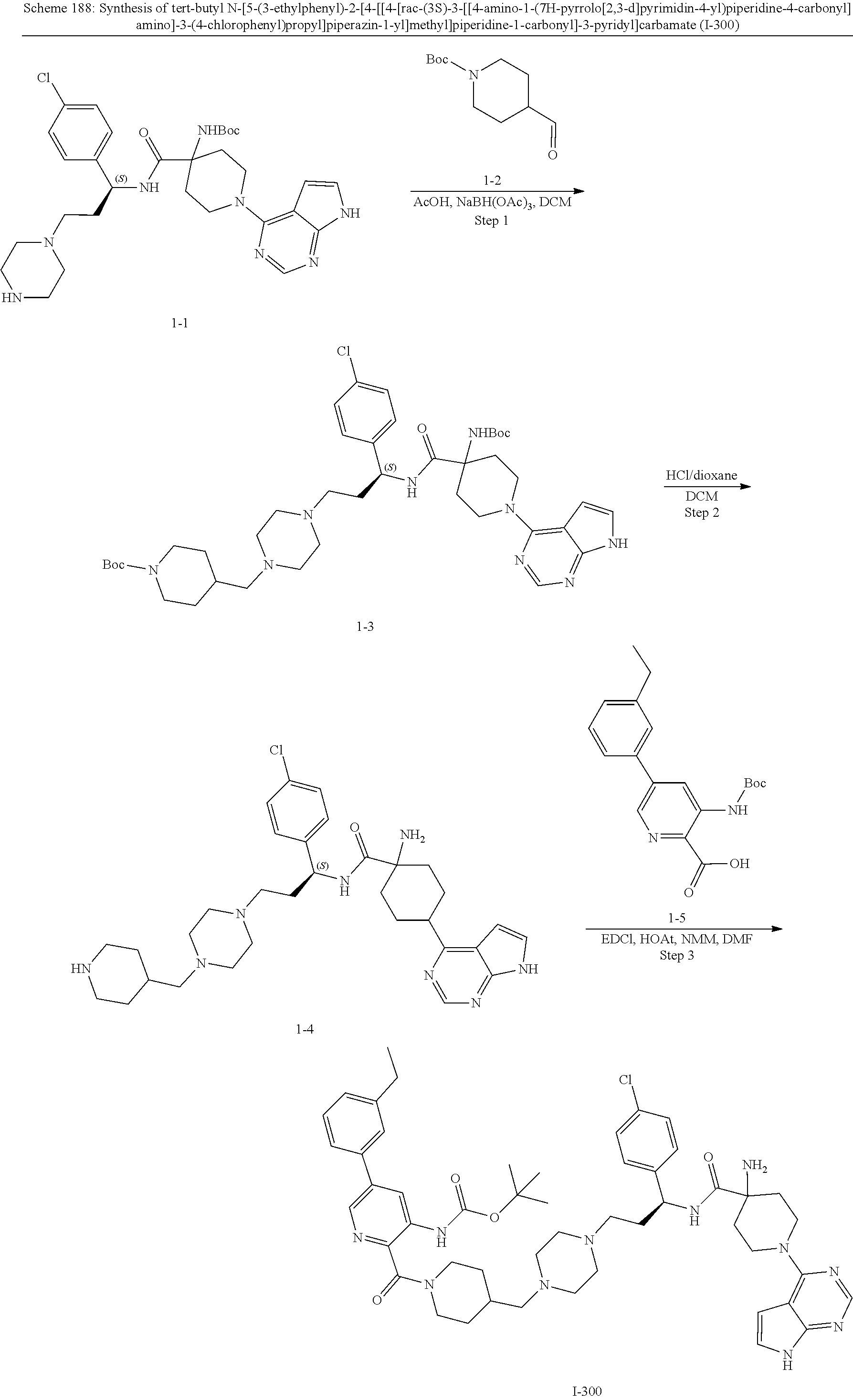

### Step 1: Synthesis of 1-3.

A mixture of 1-1 (300 mg, 502.39 μmol, 1 eq), 1-2 (128.57 mg, 602.86 μmol, 1.2 eq) in DCM (3 mL) was added AcOH (60.34 mg, 1.00 mmol, 57.52 μL, 2 eq) and NaBH(OAc)₃ (532.38 mg, 2.51 mmol, 5 eq), then the mixture was stirred at 20° C. for 5 min. The reaction mixture was diluted with water 80 mL and extracted with DCM 60 mL (30 mL*2). The combined organic layers were concentrated under reduced pressure to give a residue. 1-3 (510 mg, crude) was obtained as a light yellow solid. LCMS (Method D): R^{t}=0.334 min, (M+H)=794.4. SFC: R^{t}=1.157 min

### Step 2: Synthesis of 1-4.

To a solution of 1-3 (60 mg, 75.53 μmol, 1 eq) in DCM (1 mL) was added HCl/dioxane (2 M, 188.82 μL, 5 eq). The mixture was stirred at 20° C. for 1 hour. The reaction mixture was concentrated under reduced pressure to give a residue. The crude product was used into the next step without further purification. 1-4 (50 mg, crude, HCl salt) was obtained as a white solid. LCMS: R^{t}=0.204 min, (M+H)=594.3.

### Step 3: Synthesis of 1-300

To a solution of 1-4 (50 mg, 79.28 μmol, 1 eq, HCl salt) and 1-5 (27.15 mg, 79.28 μmol, 1 eq) in DMF (1 mL) was added HOAt (10.79 mg, 79.28 μmol, 11.09 μL, 1 eq), EDCI (45.60 mg, 237.85 μmol, 3 eq) and NMM (40.10 mg, 396.41 μmol, 43.58 μL, 5 eq). Then the mixture was stirred at 25° C. for 1 hour. The reaction mixture was diluted with water 80 mL and extracted with DCM 60 mL (30 mL*2). The combined organic layers were concentrated under reduced pressure to give a residue. The crude product was purified by reverse-phase HPLC (0.1% FA condition). The eluent was concentrated under reduced pressure to remove ACN and then lyophilized to give the product. I-300 (12.9 mg, 14.04 μmol, 17.71% yield, 100% purity) was obtained as a white solid. LCMS (Method D): R^{t}=0.386 min, (M+H)=918.4. SFC: R^{t}=1.797 min. ¹H NMR (400 MHZ, DMSO-d₆) δ=11.66 (s, 1H), 9.05 (s, 1H), 8.64 (d, J=7.6 Hz, 1H), 8.57 (d, J=1.6 Hz, 1H), 8.29-8.24 (m, 2H), 8.11 (s, 1H), 7.55-7.50 (m, 2H), 7.46-7.41 (m, 1H), 7.37-7.29 (m, 6H), 7.15 (d, J=3.2 Hz, 1H), 6.58 (d, J=3.2 Hz, 1H), 4.82 (d, J=5.2 Hz, 1H), 4.49-4.35 (m, 4H), 3.04-2.98 (m, 2H), 2.82-2.62 (m, 5H), 2.54 (s, 1H), 2.35 (s, 9H), 2.21-2.17 (m, 3H), 2.09 (s, 2H), 2.01-1.97 (m, 1H), 1.96-1.92 (m, 1H), 1.90 (d, J=2.0 Hz, 1H), 1.88-1.73 (m, 6H), 1.60 (d, J=13.2 Hz, 2H), 1.41 (s, 1H), 1.26-1.15 (m, 6H), 1.11-1.03 (m, 1H).

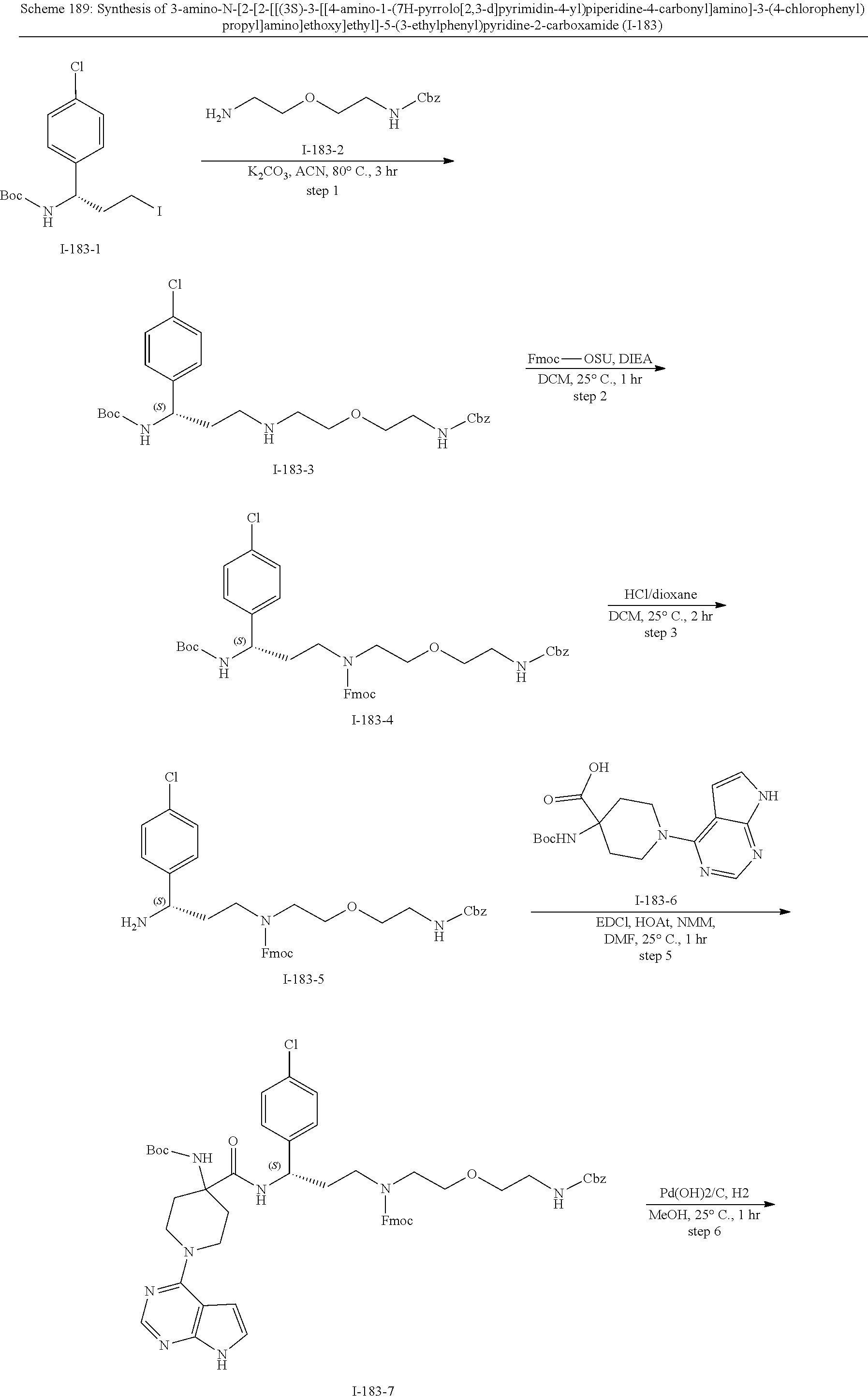

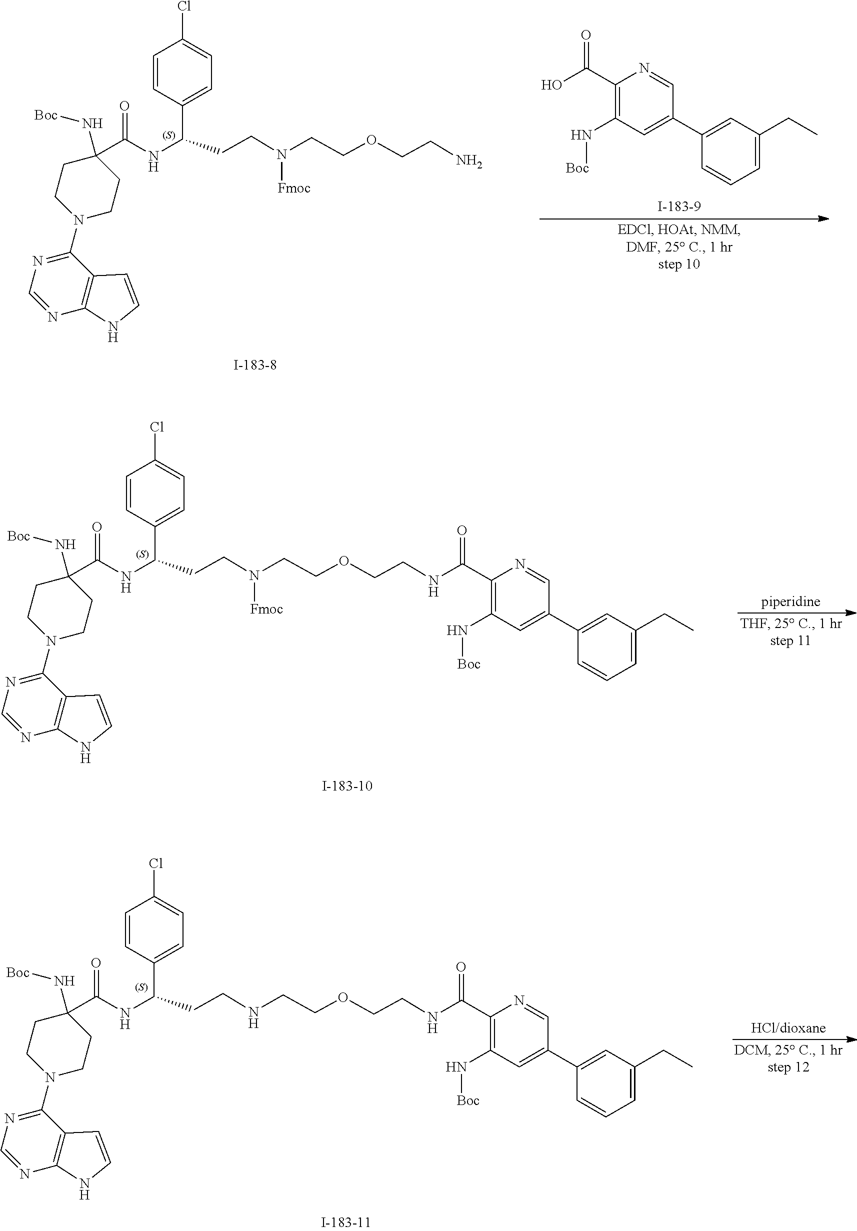

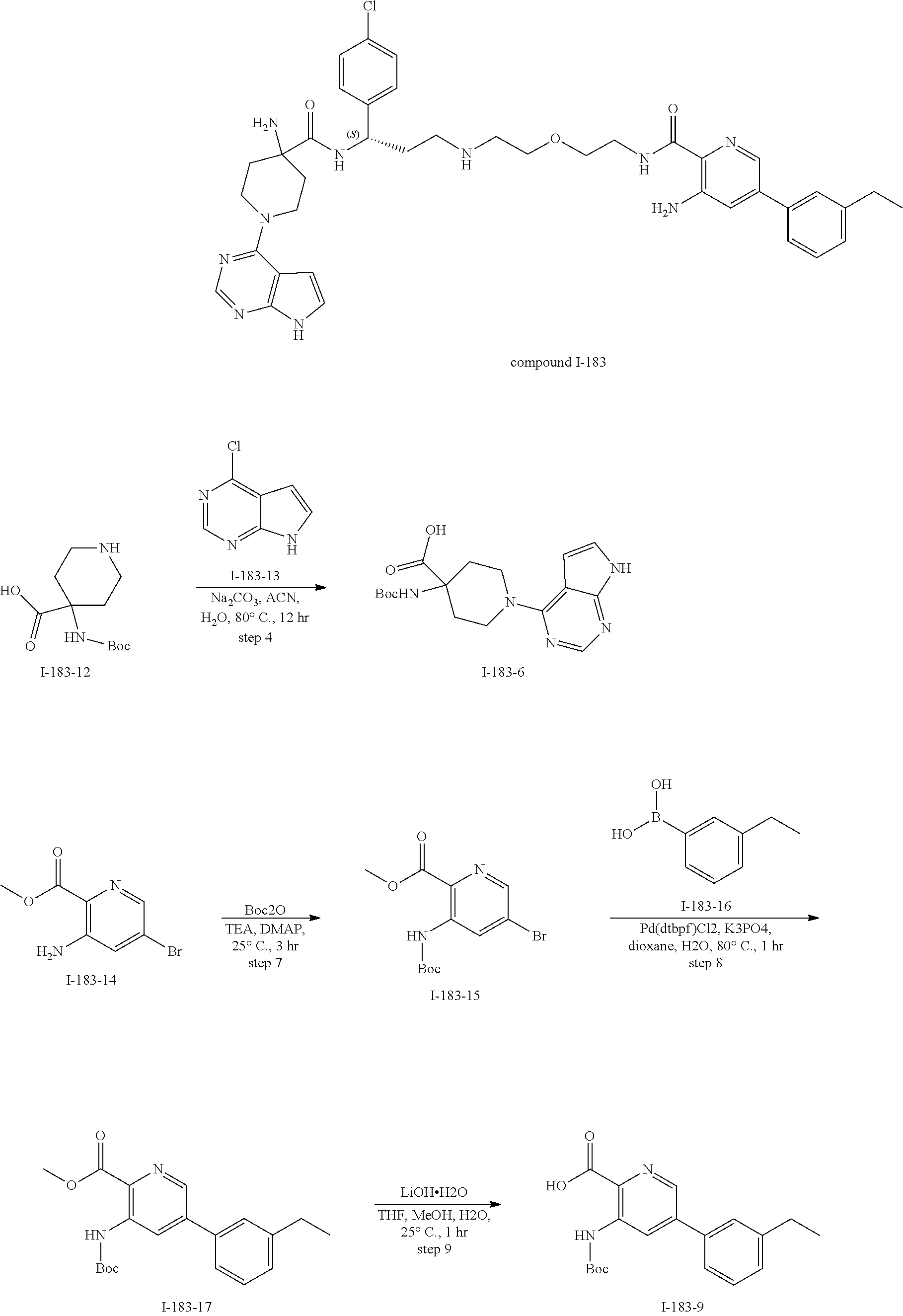

### Step 1: Synthesis of I-183-3

To a solution of I-183-1 (5 g, 12.64 mmol, 1 eq) and I-183-2 (3.01 g, 12.64 mmol, 1 eq) in ACN (50 mL) was added K₂CO₃ (5.24 g, 37.91 mmol, 3 eq). The mixture was stirred at 80° C. for 2 hrs. The mixture was washed with water (20 mL) and extract with EA (25 mL*3). The combined organic layers dried over sodium sulfate, filtered, and concentrated under reduced pressure to give a residue. The residue was purified by flash silica gel chromatography (ISCO®; 80 g SepaFlash® Silica Flash Column, Eluent of 0˜100% MeOH/EA @ 100 mL/min) and then the eluent was concentrated in vacuo to give product. I-183-3 (3.6 g, 6.40 mmol, 50.67% yield, 90% purity) was obtained as yellow oil. LCMS (Method D): R^{t}=0.385 min, (M+H)=506.2. ¹H NMR (400 MHZ, CHLOROFORM-d) δ=7.37-7.32 (m, 5H), 7.31-7.27 (m, 2H), 7.22-7.16 (m, 2H), 5.91-5.61 (m, 1H), 5.10 (s, 2H), 4.75 (d, J=6.2 Hz, 1H), 3.58 (s, 2H), 3.55-3.53 (m, 2H), 3.50 (s, 2H), 3.44-3.37 (m, 2H), 2.83-2.75 (m, 2H), 2.73-2.64 (m, 2H), 1.88-1.78 (m, 2H), 1.47-1.37 (m, 9H).

### Step 2: Synthesis of I-183-4

To a solution of I-183-3 (3.6 g. 7.11 mmol, 1 eq) in DCM (36 mL) was added Fmoc-OSu (2.88 g, 8.54 mmol, 1.2 eq) and DIEA (2.76 g, 21.34 mmol, 3.72 mL, 3 eq). The mixture was stirred at 25° C. for 1 hr. The reaction mixture was concentrated in vacuo. The residue was purified by flash silica gel chromatography (ISCO®; 80 g SepaFlash® Silica Flash Column, Eluent of 0˜50% EA/PE @ 100 mL/min) and then the eluent was concentrated in vacuo to give product. I-183-4 (4.5 g, 6.12 mmol, 85.99% yield, 99% purity) was obtained as colorless gum, and. LCMS (Method D): R^{t}=0.571 min, (M+H)=728.3. SFC: R^{t}=1.563 min. ¹H NMR (400 MHZ, CHLOROFORM-d) δ=7.73 (s, 2H), 7.54 (d, J=7.2 Hz, 2H), 7.40-7.35 (m, 5H), 7.34-7.29 (m, 3H), 7.27-7.23 (m, 3H), 7.19 (s, 1H), 7.04 (s, 1H), 5.11 (s, 2H), 4.70-4.49 (m, 3H), 4.22-4.12 (m, 1H), 3.46 (s, 2H), 3.31 (s, 4H), 3.23-2.80 (m, 4H), 1.92 (d, J=1.6 Hz, 1H), 1.60-1.53 (m, 1H), 1.50-1.39 (m, 9H).

### Step 3: Synthesis of I-183-5

To a solution of I-183-4 (3.4 g, 4.67 mmol, 1 eq) in DCM (20 mL) was added HCl/dioxane (2 M, 20 mL). The mixture was stirred at 25° C. for 2 hr. The reaction mixture was concentrated in vacuo. The crude product was used into the next step without further purification. I-183-5 (3.4 g, crude, HCl salt) was obtained as yellow gum. LCMS (Method D): R^{t}=0.422 min, (M+H)=628.6.

### Step 4: Synthesis of I-183-6

To a solution of I-183-13 (3.14 g, 20.47 mmol, 1 eq) and I-183-12 (5 g. 20.47 mmol, 1 eq) in ACN (20 mL) and H₂O (60 mL) was added Na₂CO₃ (10.85 g, 102.34 mmol, 5 eq). The mixture was stirred at 80° C. for 12 hr. The aqueous layer was concentrated under oil pump and methanol (30 mL) was added. The mixture was stirred at 25° C. for 30 min and filtered. The filtrate was concentrated and dissolved into water (30 mL), 4M HCl (˜4 mL) was added into stirred solution to adjusted pH=4, and white solid was formed. The suspension was filtered to afford yellow solid. The crude product was used into the next step without further purification. I-183-6 (3.5 g. 9.20 mmol, 44.95% yield, 95% purity) was obtained as a yellow solid. LCMS (Method D): R^{t}=0.242 min, (M+H)=362.2. ¹H NMR (400 MHz, DMSO-d₆) δ=11.80 (s, 1H), 8.15 (s, 1H), 7.19 (d, J=2.0 Hz, 1H), 6.65-6.61 (m, 1H), 4.30 (d, J=12.8 Hz, 2H), 3.51-3.48 (m, 2H), 2.03 (s, 2H), 1.96-1.87 (m, 2H), 1.39 (s, 9H).

### Step 5: Synthesis of I-183-7

To a solution of I-183-6 (1.79 g, 4.97 mmol, 1 eq) in DMF (30 mL) was added EDCI (2.86 g, 14.90 mmol, 3 eq), HOAt (675.82 mg, 4.97 mmol, 694.58 μL, 1 eq) and NMM (2.51 g, 24.83 mmol, 2.73 mL, 5 eq). Then I-183-5 (3.3 g. 4.97 mmol, 1 eq, HCl) was added. The mixture was stirred at 25° C. for 1 hr. The mixture was washed with water (30 mL) and extract with EA (20 mL*3). The combined organic layers dried over sodium sulfate, filtered, and concentrated under reduced pressure to give a residue. The residue was purified by flash silica gel chromatography (ISCO®; 80 g SepaFlash® Silica Flash Column, Eluent of 0˜50% MeOH/EA @ 100 mL/min) and then the eluent was concentrated in vacuo to give product. I-183-7 (4.4 g, 4.44 mmol, 89.39% yield, 98% purity) was obtained as a white solid. LCMS (Method D): R^{t}=0.445 min, (M+H)=971.2. SFC: R^{t}=2.034 min. ¹H NMR (400 MHZ, DMSO-d₆) δ=11.65 (s, 1H), 8.11 (s, 1H), 7.95 (s, 1H), 7.88-7.81 (m, 2H), 7.58-7.55 (m, 2H), 7.37-7.25 (m, 12H), 7.22-7.09 (m, 3H), 6.98 (s, 1H), 6.55 (d, J=15.6 Hz, 1H), 4.99 (s, 2H), 4.41 (s, 2H), 4.21 (d, J=15.2 Hz, 3H), 3.62-3.46 (m, 4H), 3.25-3.15 (m, 3H), 3.08 (s, 6H), 1.97 (s, 4H), 1.88-1.70 (m, 2H), 1.36 (s, 9H).

### Step 6: Synthesis of 8

To a solution of I-183-7 (200 mg, 205.86 μmol, 1 eq) in MeOH (2 mL) was added Pd(OH): (50 mg, 71.21 μmol, 20% purity; 3.46e-1 eq) under N₂ atmosphere. The suspension was degassed and purged with H2 (414.97 μg, 205.86 μmol, 1 eq) for 3 times. The mixture was stirred under H₂ (15 Psi) at 25° C. for 1 hr. The resulting mixture was filtered, and the filtrate was concentrated under reduced pressure. The crude product was used into the next step without further purification. I-183-8 (120 mg, crude) was obtained as yellow oil. LCMS (Method D): R^{t}=0.388 min, (M+H)=837.3.

### Step 7: Synthesis of I-183-15

To a solution of I-183-14 (10 g, 43.28 mmol, 1 eq) in DCM (100 mL) was added TEA (13.14 g, 129.84 mmol, 18.07 mL, 3 eq), DMAP (528.76 mg, 4.33 mmol, 0.1 eq) and Boc₂O (10.39 g, 47.61 mmol, 10.94 mL, 1.1 eq). The mixture was stirred at 25° C. for 3 hr. The mixture was washed with water (100 mL) and extract with EA (100 mL*2). The combined organic layers dried over sodium sulfate, filtered, and concentrated under reduced pressure to give a residue. The residue was purified by flash silica gel chromatography (ISCO®; 120 g SepaFlash® Silica Flash Column, Eluent of 0˜20% EA/PE @ 100 mL/min) and then the eluent was concentrated in vacuo to give product. I-183-15 (12 g, 33.34 mmol, 77.02% yield, 92% purity) was obtained as a white solid. LCMS (Method D): R^{t}=0.417 min, (M+H)=332.9. ¹H NMR (400 MHZ, CHLOROFORM-d) δ=10.20 (s, 1H), 9.13 (s, 1H), 8.36 (s, 1H), 4.01 (s, 3H), 1.53 (s, 9H).

### Step 8: Synthesis of I-183-17

A mixture of I-183-15, I-183-16 (6.52 g, 43.48 mmol, 1.2 eq), Pd(dtbpf)Cl₂ (2.36 g, 3.62 mmol, 0.1 eq) and K₃PO₄ (23.08 g, 108.71 mmol, 3 eq) in dioxane (100 mL) and H₂O (20 mL) was degassed and purged with N₂ for 3 times, and then the mixture was stirred at 80° C. for 1 hr under N₂ atmosphere. The mixture was washed with water (100 mL) and extract with EA (150 mL*2). The combined organic layers dried over sodium sulfate, filtered, and concentrated under reduced pressure to give a residue. The residue was purified by flash silica gel chromatography (ISCO®; 120 g SepaFlash® Silica Flash Column, Eluent of 0˜50% EA/PE @ 100 mL/min) and then the eluent was concentrated in vacuo to give product. I-183-17 (11 g, 29.01 mmol, 80.06% yield, 94% purity) was obtained as yellow oil. LCMS (Method D): R^{t}=0.527 min, (M+H)=357.2. ¹H NMR (400 MHZ, CHLOROFORM-d) δ=10.28 (s, 1H), 9.10 (s, 1H), 8.58 (s, 1H), 7.48 (s, 2H), 7.44-7.39 (m, 1H), 7.29 (d, J=7.6 Hz, 1H), 4.05 (s, 3H), 2.75-2.74 (m, 2H), 1.56 (s, 9H), 1.30-1.28 (m, 3H).

### Step 9: Synthesis of I-183-9

To a solution of I-183-17 00 mg, 1.40 mmol, 1 eq) in THF (2 mL), MeOH (2 mL) and H₂O (2 mL) was added LiOH·H₂O (176.61 mg, 4.21 mmol, 3 eq). The mixture was stirred at 25° C. for 1 hr. The pH was acidified to 5˜6 with 1N HCl, and the mixture was washed with water (5 mL) and extract with EA (8 mL*3). The combined organic layers dried over sodium sulfate, filtered, and concentrated under reduced pressure to give a residue. The crude product was used into the next step without further purification. I-183-9 (450 mg, 1.31 mmol, 93.69% yield, 100% purity) was obtained as a brown solid. LCMS (Method D): R^{t}=0.423 min, (M+H)=343.1. ¹H NMR (400 MHZ, DMSO-d₆) δ=10.58 (s, 1H), 8.90 (d, J=1.6 Hz, 1H), 8.60 (d, J=2.0 Hz, 1H), 7.59-7.51 (m, 2H), 7.46-7.44 (m, 1H), 7.35 (d, J=7.6 Hz, 1H), 2.71-2.69 (m, 2H), 1.50 (s, 9H), 1.23-1.21 (m, 3H).

### Step 10: Synthesis of I-183-10

To a solution of I-183-8 (120 mg, 143.30 μmol, 1 eq) and I-183-9 (49.06 mg, 143.30 μmol, 1 eq) in DMF (1.2 mL) was added EDCI (82.41 mg, 429.90 μmol, 3 eq), HOAt (19.50 mg, 143.30 μmol, 20.05 μL, 1 eq) and NMM (72.47 mg, 716.50 μmol, 78.77 μL, 5 eq). The mixture was stirred at 25° C. for 1 hr. The mixture was washed with water (2 mL) and extract with EA (3 mL*3). The combined organic layers dried over sodium sulfate, filtered, and concentrated under reduced pressure to give a residue. The residue was purified by flash silica gel chromatography (ISCO®; 20 g SepaFlash® Silica Flash Column, Eluent of 0˜10% MeOH/EA @ 40 mL/min) and then the eluent was concentrated in vacuo to give product. I-183-10 (70 mg, 60.25 μmol, 42.05% yield, 100% purity) was obtained as a white solid. LCMS (Method D): R^{t}=0.566 min, (M+H)=1162.6. SFC: R^{t}=2.143 min. ¹H NMR (400 MHZ, METHANOL-d4) δ=8.89 (s, 1H), 8.32 (s, 1H), 8.12-8.03 (m, 1H), 7.73 (d, J=5.2 Hz, 1H), 7.56 (d, J=6.8 Hz, 2H), 7.47-7.42 (m, 2H), 7.41-7.36 (m, 2H), 7.33-7.15 (m, 10H), 7.08-7.03 (m, 1H), 6.57-6.46 (m, 1H), 4.83-4.67 (m, 2H), 4.51 (d, J=9.6 Hz, 2H), 4.37-4.27 (m, 2H), 4.20-4.11 (m, 1H), 3.60-3.53 (m, 3H), 3.49-3.42 (m, 4H), 3.21-3.11 (m, 4H), 2.71-2.70 (m, 2H), 2.13-1.96 (m, 5H), 1.79-1.70 (m, 1H), 1.54 (s, 9H), 1.38 (s, 9H), 1.29-1.25 (m, 3H).

### Step 11: Synthesis of I-183-11

To a solution of I-183-10 (50 mg, 43.04 μmol, 1 eq) in THF (0.8 mL) was added piperidine (86.22 mg, 1.01 mmol, 0.1 mL, 23.53 eq). The mixture was stirred at 25° C. for 0.5 hr. The reaction mixture was concentrated in vacuo. The crude product was used into the next step without further purification. I-183-11 (40 mg, crude) was obtained as yellow oil. LCMS (Method D): R^{t}=0.438 min, (M+H)=939.3.

### Step 11: Synthesis of I-183

To a solution of I-183-11 (40 mg, 42.57 μmol, 1 eq) in DCM (0.2 mL) was added HCl/dioxane (2 M, 0.2 mL). The mixture was stirred at 25° C. for 0.5 hr. The reaction mixture was concentrated in vacuo. The residue was purified by flash silica gel chromatography (ISCO®; 12 g SepaFlash® Silica Flash Column, Eluent of 0˜100% MeOH (0.2% NH₃·H₂O)/DCM (30 mL/min) and then the eluent was concentrated in vacuo to give product. Then the residue was purified by prep-HPLC (column: CD01-Phenomenex luna C18 150*25*10 um; mobile phase: [water (FA)-ACN]; gradient: 12%-42% B over 8 min) and the eluent was concentrated under reduced pressure to remove ACN and lyophilized to give product. I-183 (6.02 mg, 7.67 μmol, 18.01% yield, 100% purity, FA salt) was obtained as a yellow solid. LCMS (Method D): R^{t}=0.377 min, (M+H)=739.4. SFC: R^{t}=0.869 min. ¹H NMR (400 MHZ, METHANOL-d4) δ=8.40 (s, 2H), 8.12 (s, 1H), 8.00 (d, J=2.0 Hz, 1H), 7.39 (s, 1H), 7.36 (d, J=7.2 Hz, 1H), 7.34-7.33 (m, 2H), 7.30 (d, J=2.4 Hz, 3H), 7.29-7.25 (m, 2H), 7.11 (d, J=3.6 Hz, 1H), 6.58 (d, J=3.6 Hz, 1H), 4.99-4.98 (m, 1H), 4.49 (d, J=14.0 Hz, 1H), 4.45-4.40 (m, 1H), 3.76-3.74 (m, 2H), 3.70-3.66 (m, 2H), 3.64-3.61 (m, 2H), 3.61-3.57 (m, 1H), 3.53-3.48 (m, 1H), 3.26-3.21 (m, 2H), 3.16-3.11 (m, 1H), 3.02-2.95 (m, 1H), 2.71-2.69 (m, 2H), 2.28-2.22 (m, 1H), 2.22-2.18 (m, 2H), 2.18-2.14 (m, 1H), 1.63 (d, J=13.6 Hz, 1H), 1.54 (d, J=14.0 Hz, 1H), 1.27-1.26 (m, 3H).

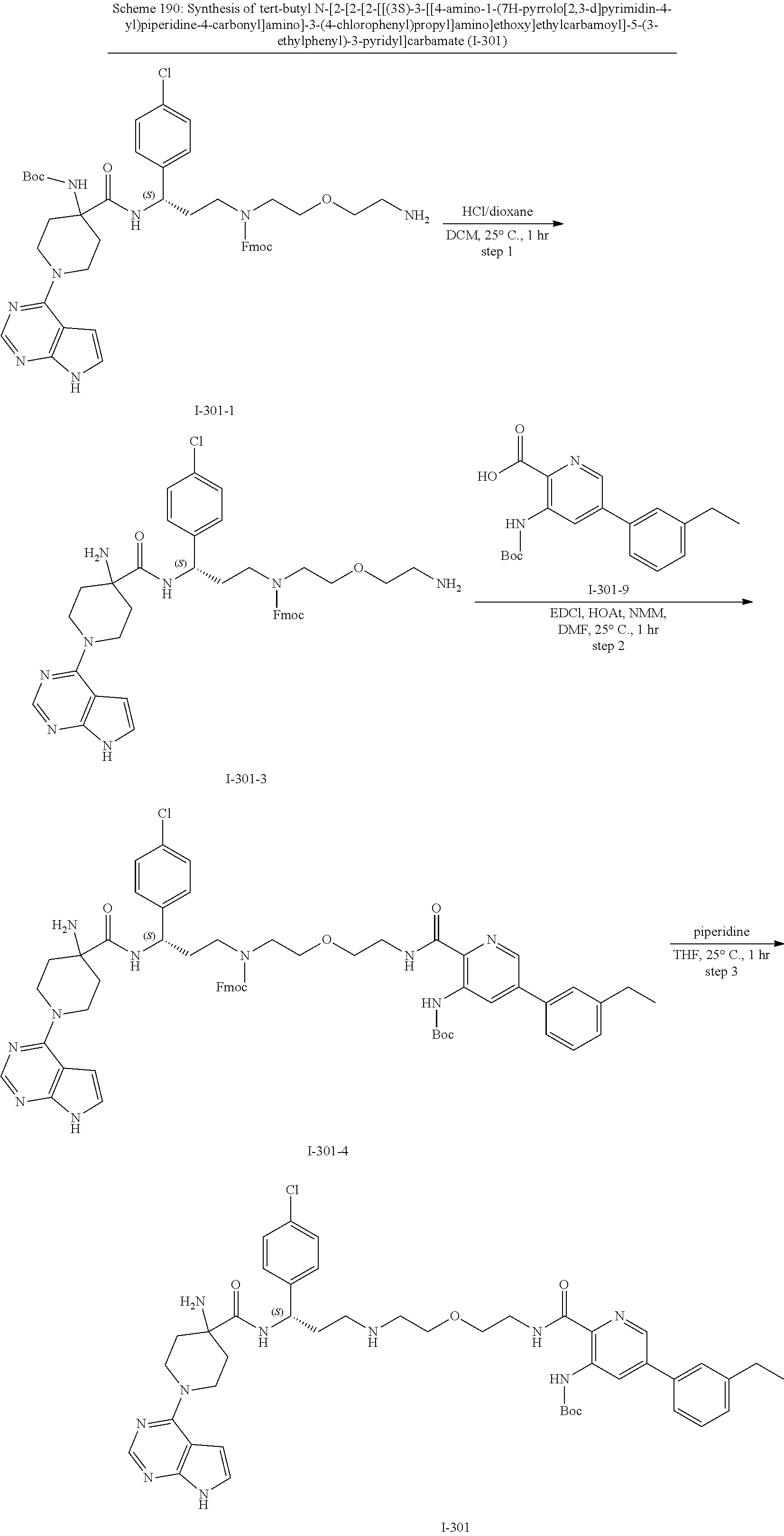

### Step 1: Synthesis of I-301-2

To a solution of I-301-1 (200 mg, 238.83 μmol, 1 eq) in DCM (2 mL) was added HCl/dioxane (2 M, 2 mL). The mixture was stirred at 25° C. for 1 hr. The reaction mixture was concentrated in vacuo. The crude product was used into the next step without further purification. I-301-2 (200 mg, crude, HCl salt) was obtained as a white solid. LCMS (Method D): R^{t}=0.339 min, (M+H)=737.4.

### Step 2: Synthesis of I-301-4

To a solution of I-301-3 (88.50 mg, 258.48 μmol, 1 eq) in DMF (2 mL) was added EDCI (148.65 mg, 775.45 μmol, 3 eq), HOAt (35.18 mg, 258.48 μmol, 36.16 μL, 1 eq) and NMM (130.72 mg, 1.29 mmol, 142.09 μL, 5 eq). Then I-301-2 (200 mg, 258.48 μmol, 1 eq, HCl salt) was added. The result mixture was stirred at 25° C. for 1 hr. The mixture was washed with water (3 mL) and extract with EA (5 mL*3). The combined organic layers dried over sodium sulfate, filtered, and concentrated under reduced pressure to give a residue. The residue was purified by flash silica gel chromatography (ISCO®; 20 g SepaFlash® Silica Flash Column, Eluent of 0˜100% MeOH/EA @ 60 mL/min) and then the eluent was concentrated in vacuo to give product. I-301-3 (130 mg, 85.71 μmol, 33.16% yield, 70% purity) was obtained as yellow oil. LCMS (Method D): R^{t}=0.496 min, (M+H)=1061.2.

### Step 3: Synthesis of I-301

To a solution of I-301-3 (130 mg, 85.71 μmol, 1 eq) in THF (1.2 mL) was added piperidine (129.33 mg, 1.52 mmol, 0.15 mL, 17.72 eq). The mixture was stirred at 25° C. for 0.5 hr. The reaction mixture was concentrated in vacuo. The residue was purified by prep-HPLC (column: CD01-Phenomenex luna C18 150*25*10 um; mobile phase: [water (FA)-ACN]; gradient: 30%-60% B over 10 min) and the eluent was concentrated under reduced pressure to remove ACN and lyophilized to give product. I-301 (25.76 mg, 27.72 μmol, 32.34% yield, 95.273% purity, FA salt) was obtained as a white solid. LCMS (Method D): R^{t}=0.392 min, (M+H)=839.3. SFC: R^{t}=0.656 min. ¹H NMR (400 MHZ, METHANOL-d4) δ=8.97 (d, J=1.6 Hz, 1H), 8.58-8.43 (m, 1H), 8.37 (d, J=2.0 Hz, 1H), 8.11 (s, 1H), 7.46 (s, 1H), 7.42-7.38 (m, 2H), 7.33-7.28 (m, 5H), 7.10 (d, J=3.6 Hz, 1H), 6.56 (d, J=3.6 Hz, 1H), 4.99-4.93 (m, 1H), 4.52-4.45 (m, 1H), 4.44-4.37 (m, 1H), 3.75-3.74 (m, 2H), 3.68-3.67 (m, 2H), 3.66-3.61 (m, 2H), 3.60-3.56 (m, 1H), 3.52-3.47 (m, 1H), 3.20-3.02 (m, 2H), 3.14-3.09 (m, 1H), 2.96-2.95 (m, 1H), 2.73-2.72 (m, 2H), 2.27-2.20 (m, 1H), 2.20-2.16 (m, 2H), 2.12 (s, 1H), 1.60 (d, J=14.0 Hz, 1H), 1.54 (s, 9H), 1.49 (d, J=14.8 Hz, 1H), 1.28-1.26 (m, 3H).

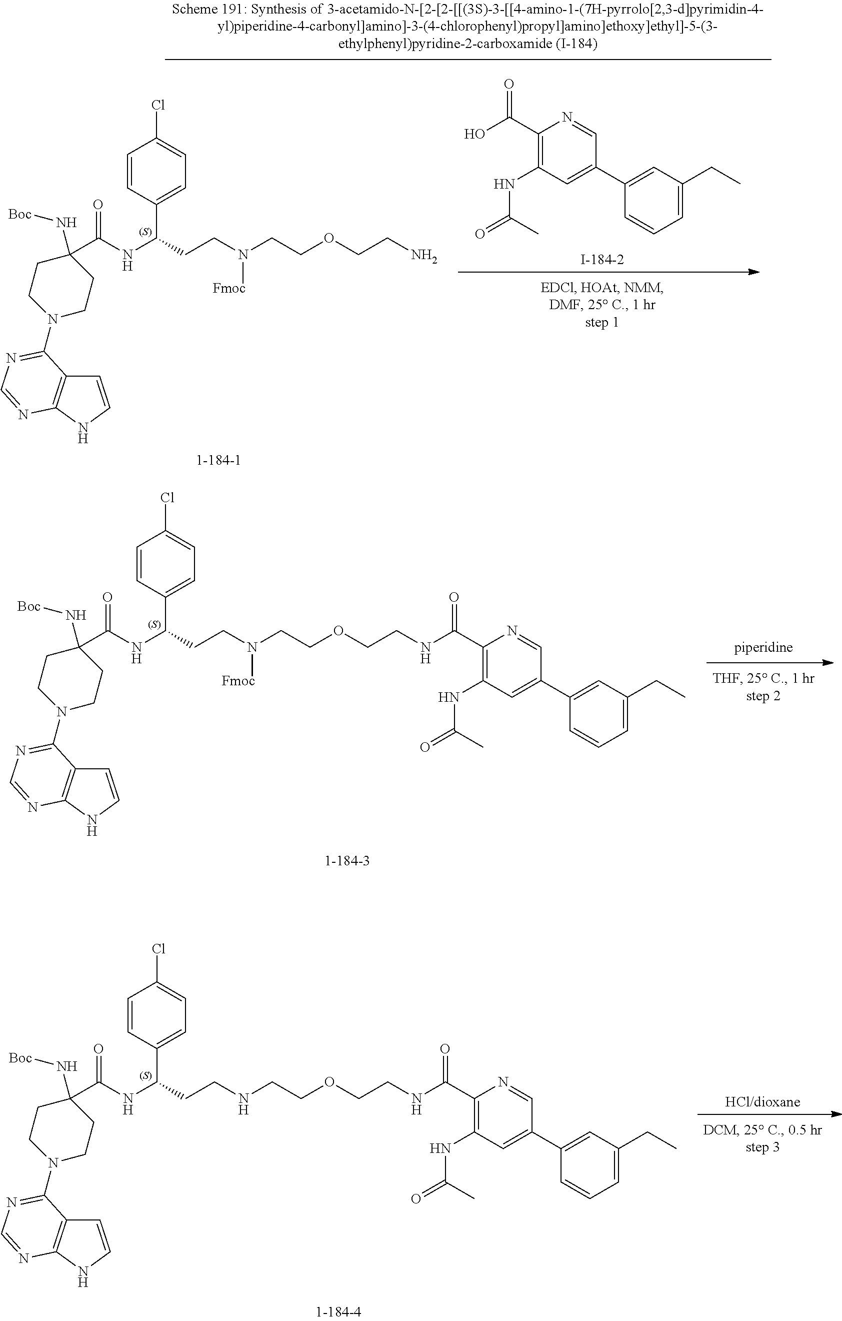

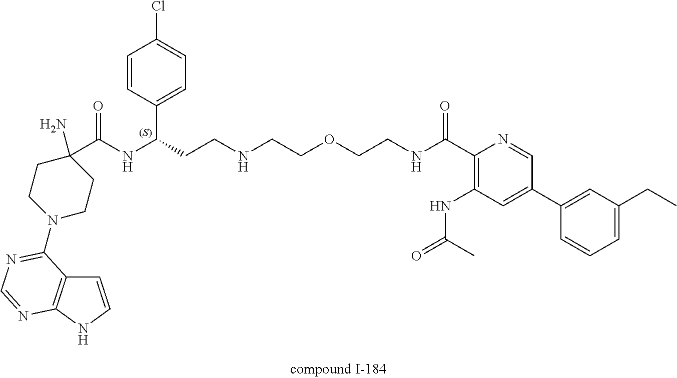

### Step 1: Synthesis of I-184-3

To a solution of I-184-1 (250 mg, 298.54 μmol, 1 eq) and I-184-2 (84.88 mg, 298.54 μmol, 1 eq) in DMF (3 mL) was added EDCI (171.69 mg, 895.63 μmol, 3 eq), HOAt (40.63 mg, 298.54 μmol, 41.76 μL, 1 eq) and NMM (150.98 mg, 1.49 mmol, 164.11 μL, 5 eq). The mixture was stirred at 25° C. for 1 hr. The mixture was washed with water (3 mL) and extract with EA (5 mL*3). The combined organic layers dried over sodium sulfate, filtered, and concentrated under reduced pressure to give a residue. The residue was purified by flash silica gel chromatography (ISCO®; 12 g SepaFlash® Silica Flash Column, Eluent of 0˜100% MeOH/EA @ 40 mL/min) and then the eluent was concentrated in vacuum to give product. I-184-3 (90 mg, 79.10 μmol, 26.49% yield, 97% purity) was obtained as a yellow solid. LCMS (Method D): R^{t}=0.524 min, (M+H)=1103.5. SFC: R^{t}=2.159 min.

### Step 2: Synthesis of I-184-4

To a solution of I-184-3 (80 mg, 72.48 μmol, 1 eq) in THF (0.8 mL) was added piperidine (86.22 mg, 1.01 mmol, 0.1 mL, 13.97 eq). The mixture was stirred at 25° C. for 0.5 hr. The reaction mixture was concentrated in vacuo. The crude product was used into the next step without further purification. I-184-4 (80 mg, crude) was obtained as yellow oil. LCMS (Method D): R^{t}=0.407 min, (M+H)=881.5.

### Step 3: Synthesis of I-184

To a solution of I-184-4 (80 mg, 90.76 μmol, 1 eq) in DCM (0.8 mL) was added HCl/dioxane (2 M, 0.8 mL). The mixture was stirred at 25° C. for 0.5 hr. The reaction mixture was concentrated in vacuo. The residue was purified by prep-HPLC (column: CD01-Phenomenex luna C18 150*25*10 um; mobile phase: [water (FA)-ACN]; gradient: 7%-37% B over 10 min) and the eluent was concentrated under reduced pressure to remove ACN and lyophilized to give product. I-184 (8.19 mg, 9.77 μmol, 10.77% yield, 98.731% purity, FA salt) was obtained as a white solid. LCMS (Method D): R^{t}=0.338 min, (M+H)=781.2. SFC: R^{t}=1.762 min. ¹H NMR (400 MHZ, METHANOL-d4) δ=9.19 (d, J=2.0 Hz, 1H), 8.44 (d, J=2.0 Hz, 1H), 8.37 (s, 2H), 8.11 (s, 1H), 7.45 (s, 1H), 7.42-7.38 (m, 2H), 7.33 (d, J=2.8 Hz, 1H), 7.31 (d, J=1.6 Hz, 4H), 7.10 (d, J=3.6 Hz, 1H), 6.56 (d, J=3.6 Hz, 1H), 4.98-4.96 (m, 1H), 4.48-4.43 (m, 1H), 4.40-4.34 (m, 1H), 3.77-3.76 (m, 2H), 3.71-3.70 (m, 2H), 3.68-3.61 (m, 3H), 3.57-3.51 (m, 1H), 3.27-3.22 (m, 2H), 3.21-3.09 (m, 2H), 3.03-2.96 (m, 1H), 2.73-2.71 (m, 2H), 2.31-2.24 (m, 1H), 2.22 (s, 3H), 2.20 (s, 1H), 2.19-2.17 (m, 1H), 1.65 (d, J=14.0 Hz, 1H), 1.56 (d, J=13.6 Hz, 1H), 1.28-1.26 (m, 3H).

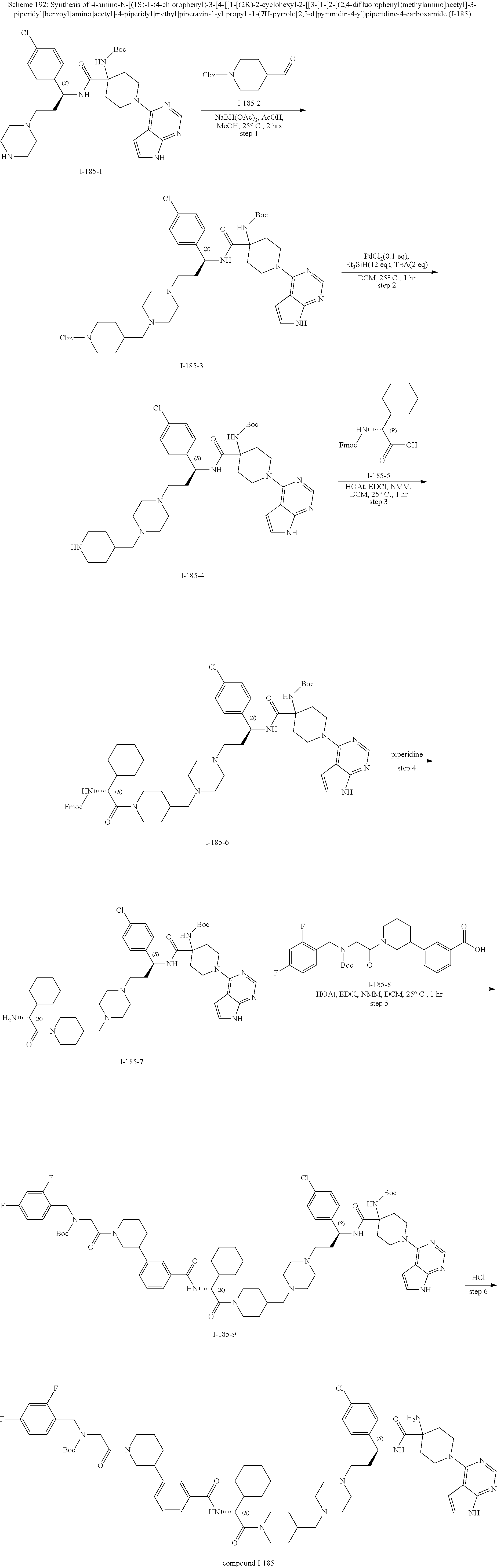

### Step 1: Synthesis of I-185.3

To a solution of I-185-1 (350 mg, 586.12 μmol, 1 eq) and I-185-2 (144.94 mg, 586.12 μmol, 1 eq) in MeOH (3 mL) was added AcOH (35.20 mg, 586.12 μmol, 33.55 μL, 1 eq). The mixture was stirred at 25° C. for 0.5 hr. Then NaBH(OAc)₃ (248.44 mg, 1.17 mmol, 2 eq) was added into the mixture and stirred at 25° C. for 1.5 hr. The reaction mixture was diluted with water (30 mL) and extracted with ethyl acetate (10 mL×3). The combined organic layers were washed with brine (10 mL×3) and dried over sodium sulfate, filtered, and concentrated under reduced pressure to give a residue. The residue was purified by flash silica gel chromatography (ISCO®; 20 g SepaFlash R: Silica Flash Column, Eluent of 0˜10% MeOH: DCM @ 40 mL/min). The eluent was concentrated under reduced pressure to give I-185-3 (440 mg, 437.37 μmol, 74.62% yield, 82.349% purity) as a yellow oil. LCMS: R^{t}=0.456 min, (M+H)=828.4.

### Step 2: Synthesis of I-185-4

To a solution of I-185-3 (420 mg, 506.98 μmol, 1 eq) in DCM (4 mL) was added PdCl2 (17.98 mg, 101.40 μmol, 0.2 eq) and TEA (102.60 mg, 1.01 mmol, 141.13 μL, 2 eq). The reaction mixture was degassed with N2. Then Et3SiH (294.75 mg, 2.53 mmol, 404.88 μL, 5 eq) was added. The reaction mixture was stirred at 25° C. for 1 hr. The reaction mixture was diluted with water (30 mL) and extracted with ethyl acetate (10 mL×3). The combined organic layers were washed with brine (10 mL×3) and dried over sodium sulfate, filtered, and concentrated under reduced pressure to give I-185-4 (320 mg, 357.82 μmol, 70.58% yield, 77.637% purity) as a yellow oil. LCMS (Method D): R^{t}=0.267 min, (M+H)=694.3.1

### Step 3: Synthesis of I-185-6

To a solution of I-185-5 (163.95 mg, 432.09 μmol, 163.95 μL, 1 eq) in DCM (5 mL) was added EDCI (248.49 mg, 1.30 mmol, 3 eq), HOAt (58.81 mg, 432.09 μmol, 60.44 μL, 1 eq) and NMM (218.52 mg, 2.16 mmol, 237.52 μL, 5 eq) at 25° C. for 0.5 hr. And then I-185-4 (300 mg, 432.09 μmol, 1 eq) was added into mixture and stirred at 25° C. for 0.5 hr. The reaction mixture was diluted with water (30 mL) and extracted with ethyl acetate (10 mL×3). The combined organic layers were washed with brine (10 mL×3) and dried over sodium sulfate, filtered, and concentrated under reduced pressure to give a residue. The crude product was purified by reverse-phase HPLC (neutral condition) and the eluent was concentrated under reduced pressure to remove ACN and then lyophilized to give I-185-6 (220 mg, 140.66 μmol, 32.55% yield, 67.5% purity) as a white solid. LCMS (Method D): R^{t}=0.418 min, (M+H)=1055.5.

### Step 4: Synthesis of I-185-7

To a solution of I-185-6 (220 mg, 208.38 μmol, 1 eq) in THF (2 mL) was added piperidine (3.58 mmol, 353.14 μL). The mixture was stirred at 25° C. for 1 hr. The mixture was concentrated under reduced pressure to give a residue. The crude product was purified by reverse-phase HPLC (neutral condition) and the eluent was concentrated under reduced pressure to remove ACN and then lyophilized to give I-185-7 (80 mg, 87.63 μmol, 42.05% yield, 91.3% purity) as a white solid. LCMS (Method D): R^{t}=0.293 min, (M+H)=833.4. ¹H NMR (400 MHZ, METHANOL-d4) δ=8.13 (s, 1H), 7.37-7.26 (m, 4H), 7.12 (s, 1H), 6.62 (s, 1H), 4.96 (s, 1H), 4.52 (d, J=12.8 Hz, 1H), 4.46-4.25 (m, 3H), 4.03-3.94 (m, 1H), 3.66-3.60 (m, 2H), 3.22-3.01 (m, 2H), 2.56-2.42 (m, 6H), 2.17 (d, J=8.8 Hz, 6H), 1.77 (d, J=13.2 Hz, 6H), 1.70-1.61 (m, 5H), 1.45 (s, 9H), 1.30-1.14 (m, 9H), 1.06 (d, J=8.8 Hz, 2H). SFC: R^{t}=8.373 min, 9.823 min.

### Step 5: Synthesis of I-185-9

To a solution of I-185-8 (11.72 mg, 24.00 μmol, 1 eq) in DCM (0.2 mL) was added EDCI (13.80 mg, 71.99 μmol, 3 eq), NMM (12.14 mg, 119.98 μmol, 13.19 μL, 5 eq) and HOAt (3.27 mg, 24.00 μmol, 3.36 μL, 1 eq) at 25° C. for 0.5 hr. And then I-185-7 (20 mg, 24.00 μmol, 1 eq) was added into mixture and stirred at 25° C. for 0.5 hr. The mixture was concentrated under reduced pressure to give I-185-9 (30 mg, 22.46 μmol, 93.58% yield, 97.608% purity) as a yellow oil. LCMS: R^{t}=0.438 min, (M+H)=1303.5.

### Step 6: Synthesis of I-185

A solution of I-185-9 (10 mg, 7.67 μmol, 1 eq) was added HCl/dioxane (2 M, 100 μL). The mixture was stirred at 25° C. for 0.5 hr. The mixture was concentrated under reduced pressure to give a residue. The crude product was purified by reverse-phase HPLC (neutral condition) and the eluent was concentrated under reduced pressure to remove ACN and then lyophilized to give I-185 (2.84 mg, 2.56 μmol, 33.36% yield, 99.415% purity) as a white solid. LCMS: (R^{t}=0.315 min, (M+H)=1103.5. ¹H NMR (400 MHZ, METHANOL-d4) δ=8.13 (s, 1H), 7.79-7.68 (m, 2H), 7.51-7.39 (m, 3H), 7.32 (s, 4H), 7.12 (s, 1H), 6.99-6.89 (m, 2H), 6.62 (s, 1H), 4.92 (d, J=5.6 Hz, 2H), 4.59-4.43 (m, 5H), 4.28 (d, J=11.2 Hz, 1H), 3.89-3.79 (m, 3H), 3.70-3.56 (m, 3H), 3.55-3.42 (m, 2H), 3.21-3.08 (m, 2H), 2.85-2.65 (m, 4H), 2.49 (d, J=7.2 Hz, 6H), 2.35 (d, J=6.0 Hz, 2H), 2.22 (s, 1H), 2.15 (s, 1H), 2.09 (d, J=4.4 Hz, 1H), 2.03 (s, 1H), 1.96-1.82 (m, 6H), 1.80-1.75 (m, 3H), 1.71-1.63 (m, 3H), 1.62-1.55 (m, 2H), 1.35-1.21 (m, 5H), 1.17-0.99 (m, 4H). ¹⁹F NMR (376 MHz, METHANOL-d4) δ=−113.349, −113.349, −116.223. SFC: R^{t}=3.621 min, 4.510 min.

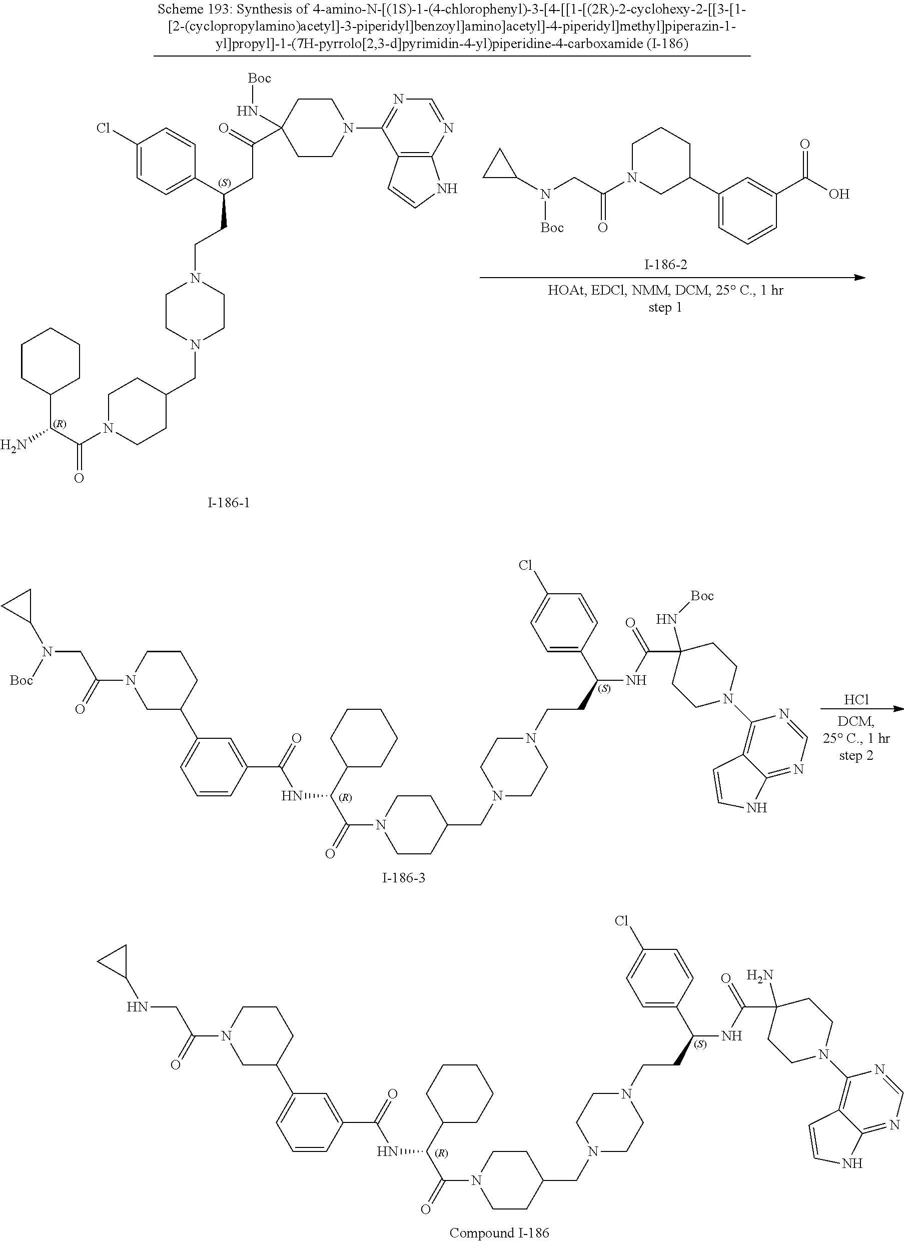

### Step 1: Synthesis of I-186-3

To a solution of I-186-2 (12.07 mg, 29.99 μmol, 1 eq) in DMF (0.2 mL) was added EDCI (17.25 mg, 89.98 μmol, 3 eq), NMM (15.17 mg, 149.97 μmol, 16.49μ, 5 eq) and HOAT (4.08 mg, 29.99 μmol, 4.20 μL, 1 eq) at 25° C. for 0.5 hr. And then I-186-1 (25 mg, 29.99 μmol, 1 eq) was added into mixture and stirred at 25° C. for 0.5 hr. The mixture was concentrated under reduced pressure to give I-186-3 (30 mg, 24.63 μmol, 82.12% yield) as a yellow oil. LCMS (Method D): R^{t}=0.423 min, (M+Na)=1239.9.

### Step 2: Synthesis of I-186

A solution of I-186-3 (20 mg, 16.42 μmol, 1 eq) in DCM (0.2 mL) was added TFA (40 μL). The mixture was stirred at 25° C. for 1 hr. The mixture was concentrated under reduced pressure to give a residue. The crude product was purified by reverse-phase HPLC (column: CD07-Daisogel SP-100-8-ODS-PK 150*25*10 um; mobile phase: [water (NH4HCO3)-ACN]; gradient: 42%-72% B over 10 min) and the eluent was concentrated under reduced pressure to remove ACN and then lyophilized to give I-186 (3.53 mg, 3.42 μmol, 20.82% yield, 98.571% purity) as a white solid. LCMS: R^{t}=0.299 min, (M+H)=1017.5. 1H NMR (400 MHZ, METHANOL-d4) δ=8.13 (s, 1H), 7.80-7.67 (m, 2H), 7.53-7.40 (m, 2H), 7.32 (s, 4H), 7.12 (s, 1H), 6.63 (s, 1H), 4.93 (d, J=5.6 Hz, 2H), 4.62-4.45 (m, 4H), 4.33-4.23 (m, 1H), 3.96-3.86 (m, 1H), 3.69-3.58 (m, 3H), 3.58-3.43 (m, 1H), 3.24-3.10 (m, 2H), 2.90-2.63 (m, 4H), 2.49 (s. 6H), 2.35 (d, J=4.8 Hz, 2H), 2.29-2.21 (m, 2H), 2.20-2.13 (m, 2H), 2.12-2.02 (m, 3H), 1.93 (s. 6H). 1.81-1.73 (m, 4H), 1.72-1.55 (m, 5H), 1.33-1.19 (m, 4H), 1.17-0.87 (m, 4H), 0.53-0.36 (m, 4H). SFC: R^{t}=3.425 min. 4.004 min.

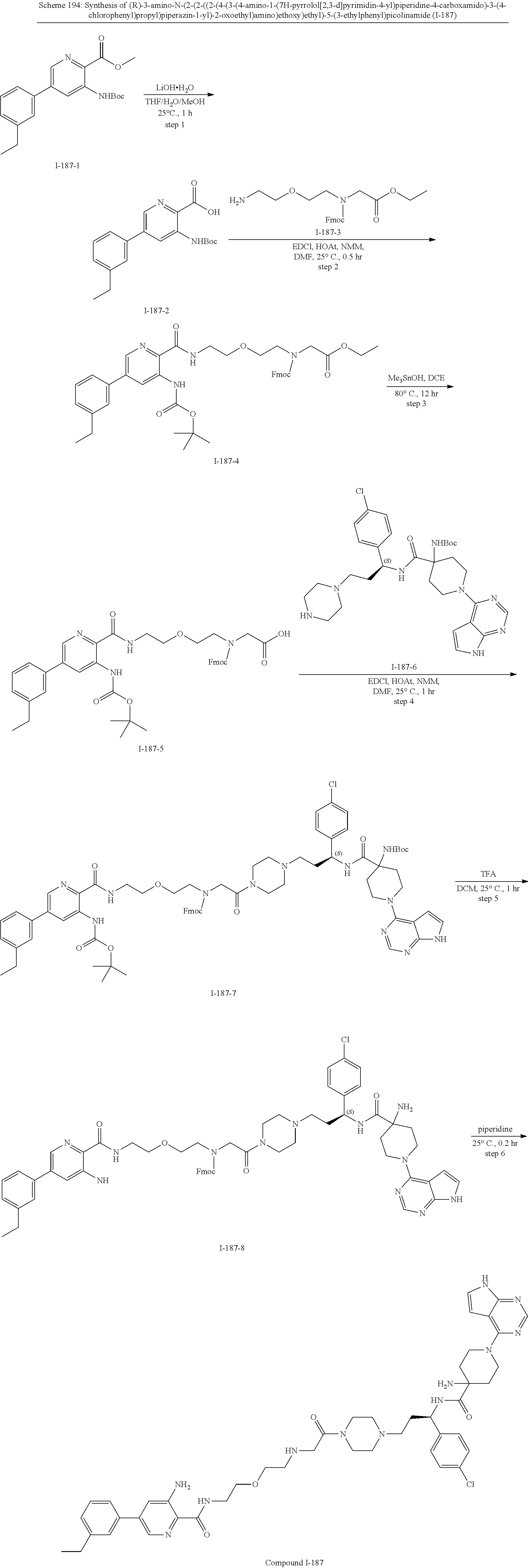

### Step 1: Synthesis of I-187-2

To a solution of I-187-1 (0.9 g, 2.53 mmol, 1 eq) in THF (3 mL), MeOH (3 mL), H₂O (3 mL) was added LiOH·H₂O (317.89 mg, 7.58 mmol, 3 eq). The mixture was stirred at 25° C. for 1 hr. The pH was acidified to 3˜4 with 1N HCl, and the mixture was washed with water (5 mL) and extract with EA (8 mL*3). The combined organic layers dried over sodium sulfate, filtered, and concentrated under reduced pressure to give a residue. The crude product was used into the next step without further purification. I-187-2 (934 mg, crude) was obtained as yellow oil. LCMS (Method D): R^{t}=0.421 min, (M+H)=343.2. ¹H NMR (400 MHz, METHANOL-d4) δ=9.19 (s, 1H), 8.55-8.42 (m, 1H), 7.56-7.51 (m, 2H), 7.46-7.41 (m, 1H), 7.34 (d, J=7.6 Hz, 1H), 2.76-2.71 (m, 2H), 1.56 (s, 9H), 1.29 (m, 3H).

### Step 2: Synthesis of I-187-4

To a solution of I-187-2 (100 mg, 292.07 μmol, 1 eq) in DMF (1 mL) was added HOAt (39.75 mg, 292.07 μmol, 40.86 μL, 1 eq), EDCI (167.97 mg, 876.20 μmol, 3 eq), NMM (147.71 mg, 1.46 mmol, 160.55 μL, 5 eq) and I-187-3 (133.91 mg, 292.07 μmol, 1 eq, FA salt). The mixture was stirred at 25° C. for 0.5 hr. The mixture was washed with water (3 mL) and extract with EA (2 mL*3). The combined organic layers dried over sodium sulfate, filtered, and concentrated under reduced pressure to give a residue. The residue was purified by reversed phase HPLC (FA condition), and the eluent was concentrated under reduced pressure to remove ACN and lyophilized to give I-187-4 (140 mg, 190.00 μmol, 65.05% yield) as yellow oil. LCMS (Method D): R^{t}=0.577 min, (M+H)=737.3. ¹H NMR (400 MHZ, METHANOL-d4) δ=9.00-8.89 (m, 1H), 8.45-8.34 (m, 1H), 7.73 (d, J=7.6 Hz, 1H), 7.67 (d, J=7.6 Hz, 1H), 7.58 (d, J=7.6 Hz, 1H), 7.49-7.40 (m, 4H), 7.39-7.22 (m, 5H), 4.48 (d, J=5.6 Hz, 1H), 4.37 (d, J=6.0 Hz, 1H), 4.23-4.13 (m, 1H), 4.13 (s, 2H), 3.96 (s, 1H), 4.03 (s, 2H), 3.65-3.52 (m, 4H), 3.52-3.44 (m, 3H), 2.76-2.67 (m, 2H), 1.55 (d, J=4.0 Hz, 9H), 1.31-1.26 (m, 3H), 1.19 (m, 3H).

### Step 3: Synthesis of I-187-5

To a solution of I-187-4 (140 mg, 190.00 μmol, 1 eq) in DCE (1.4 mL) was added Me3SnOH (171.78 mg, 949.99 μmol, 5 eq). The mixture was stirred at 80° C. for 12 hrs. The mixture was washed with aq. KF (10 mL) and diluted with water (5 mL). The mixture was stirred at 25° C. for 1 hr. Then the mixture was adjusted to pH=6˜7 with saturated citric acid aqueous solution. Then the reaction mixture was extracted with DCM (3 mL×3). The combined organic layers were washed with brine (3 mL×3) and dried over sodium sulfate, filtered, and concentrated under reduced pressure to give a residue. The crude product was used into the next step without further purification. I-187-5 (150 mg, crude) was obtained as yellow solid. LCMS (Method D): R^{t}=0.538 min, (M+H)=709.3. ¹H NMR (400 MHZ, METHANOL-d4) δ=8.91 (m, 1H), 8.44-8.31 (m, 1H), 7.70 (m, 2H), 7.58 (d, J=7.6 Hz, 1H), 7.50 (d, J=7.6 Hz, 1H), 7.44-7.26 (m, 7H), 7.23-7.19 (m, 1H), 4.44 (d, J=5.6 Hz, 1H), 4.26 (d, J=5.6 Hz, 1H), 4.20-4.06 (m, 1H), 3.99 (s, 1H), 3.88 (s, 1H), 3.78 (s, 1H), 3.66-3.63 (m, 2H), 3.59 (d, J=5.2 Hz, 1H), 3.53-3.48 (m, 3H), 3.30 (s, 2H), 2.71 (m, 2H), 1.55 (d, J=5.2 Hz, 9H), 1.30-1.25 (m, 3H).

### Step 4: Synthesis of I-187-7

To a solution of I-187-5 (50 mg, 70.54 μmol, 1 eq) in DMF (0.5 mL) was added HOAt (9.60 mg, 70.54 μmol, 9.87 μL, 1 eq), EDCI (40.57 mg, 211.63 μmol, 3 eq), NMM (35.68 mg, 352.71 μmol, 38.78 μL, 5 eq) and I-187-6 (42.12 mg, 70.54 μmol, 1 eq). The mixture was stirred at 25° C. for 1 hr. The mixture was washed with water (1 mL) and extract with EA (2 mL*3). The combined organic layers dried over sodium sulfate, filtered, and concentrated under reduced pressure to give a residue. The residue was purified by reversed phase HPLC (FA condition), the eluent was concentrated under reduced pressure to remove ACN and lyophilized to give I-187-7 (65 mg, 50.47 μmol, 71.54% yield, 100% purity) was obtained as white solid. LCMS (Method D): R^{t}=0.477 min, (M+H)=1287.5. SFC: R^{t}=0.642 min, 0.743 min.

### Step 5: Synthesis of I-187-8

To a solution of I-187-7 (55 mg, 42.70 μmol, 1 eq) in DCM (0.5 mL) was added TFA (0.1 mL). The mixture was stirred at 25° C. for 1 hr. The reaction mixture was concentrated in vacuo. The crude product was used into the next step without further purification. I-187-8 (50 mg, 41.61 μmol, 97.43% yield, TFA salt) was obtained as yellow oil. LCMS (Method D): R^{t}=0.426 min, (M/2+H)=544.7.

### Step 6: Synthesis of I-187

To a solution of I-187-8 (50 mg, 41.61 μmol, 1 eq, TFA) in THF (0.5 mL) was added piperidine (60.00 μL). The mixture was stirred at 25° C. for 0.2 hr. The reaction mixture was concentrated in vacuo. The residue was purified by reversed phase HPLC (neutral condition), the eluent was concentrated under reduced pressure to remove ACN and lyophilized to give I-187 (19.51 mg, 22.40 μmol, 53.83% yield, 99.358% purity) was obtained as white solid. LCMS (Method D): R^{t}=0.290 min, (M+H)=865.4. SFC: R^{t}=0.725 min. ¹H NMR (400 MHZ, METHANOL-d4) δ=8.10 (d, J=14.0 Hz, 2H), 7.45-7.24 (m, 9H), 7.11 (s, 1H), 6.60 (s, 1H), 4.96-4.91 (m, 1H), 4.52-4.44 (m, 2H), 3.68-3.57 (m, 10H), 3.53-3.50 (m, 2H), 3.42 (s, 2H), 2.84 (s, 2H), 2.73-2.67 (m, 2H), 2.43-2.25 (m, 6H), 2.19-2.09 (m, 2H), 2.02-1.94 (m, 1H), 1.94-1.86 (m, 1H), 1.60-1.51 (m, 2H), 1.26 (m, 3H).

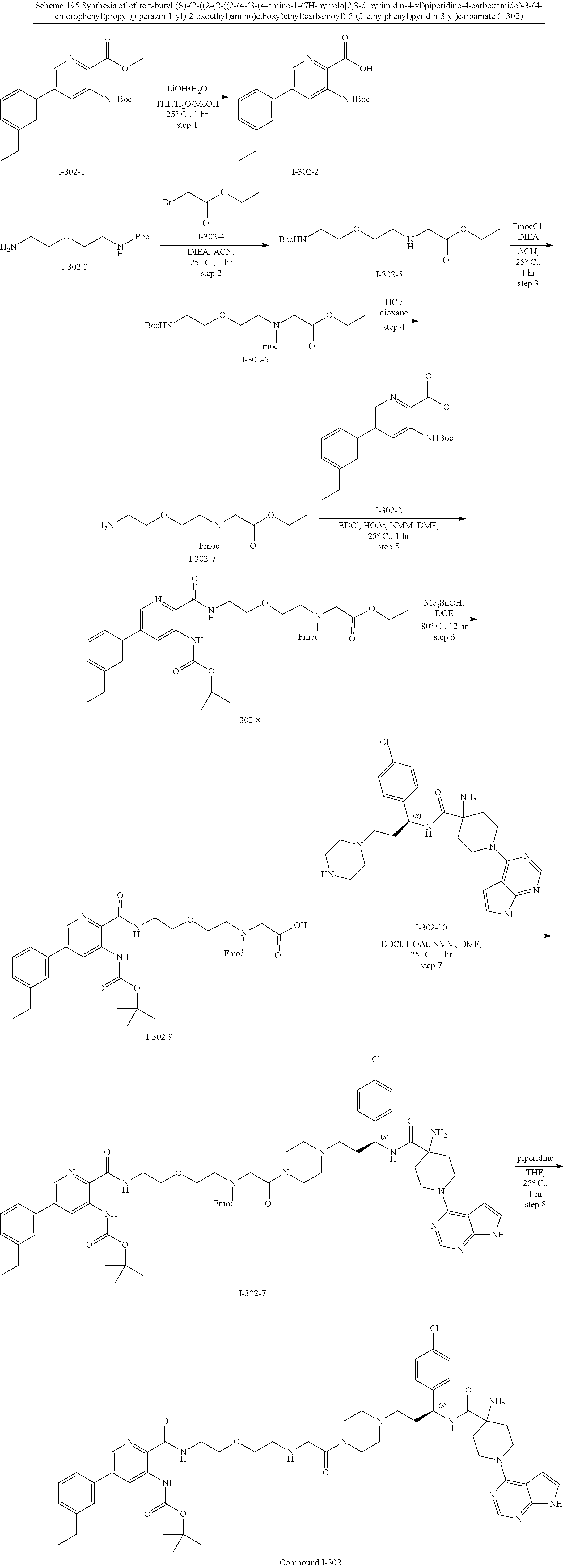

### Step 1: Synthesis of I-302-2

To a solution of I-302-1 (0.9 g, 2.53 mmol, 1 eq) in THF (3 mL), MeOH (3 mL), H₂O (3 mL) was added LiOH·H₂O (317.89 mg, 7.58 mmol, 3 eq). The mixture was stirred at 25° C. for 1 hr. The pH was acidified to 3˜4 with 1N HCl, and the mixture was washed with water (5 mL) and extract with EA (8 mL*3). The combined organic layers dried over sodium sulfate, filtered, and concentrated under reduced pressure to give a residue. The crude product was used into the next step without further purification. I-302-2 (934 mg, crude) was obtained as yellow oil. LCMS (Method D): R^{t}=0.421 min, [M+H]⁺=343.2. ¹H NMR (400 MHz, METHANOL-d4) δ=9.19 (s, 1H), 8.55-8.42 (m, 1H), 7.56-7.51 (m, 2H), 7.46-7.41 (m, 1H), 7.34 (d, J=7.6 Hz, 1H), 2.76-2.71 (m, 2H), 1.56 (s, 9H), 1.29 (m, 3H).

### Step 2: Synthesis of I-302-5

To a solution of I-302-4 (1.31 g, 7.83 mmol, 866.87 μL, 0.8 eq) in ACN (10.5 mL) was added DIEA (3.80 g, 29.37 mmol, 5.12 mL, 3 eq) and I-302-3 (2 g, 9.79 mmol, 1 eq). The mixture was stirred at 25° C. for 1 hr. The reaction mixture was concentrated under reduced pressure to give a residue and used into the next step without further purification. I-302-5 (2.6 g, crude) was obtained as a brown oil. LCMS (Method D): R^{t}=0.139 min, [M+H]⁺=291.1.

### Step 3: Synthesis of I-302-6

To a solution of I-302-5 (2.5 g, 8.61 mmol, 1 eq) in ACN (10 mL) was added DIEA (1.11 g, 8.61 mmol, 1.50 mL, 1 eq) and Fmoc-Cl (2.67 g, 10.33 mmol, 1.2 eq). The mixture was stirred at 25° C. for 1 hr. The reaction mixture was diluted with H₂O 5 mL and extracted with EA (5 mL*3). The combined organic layers were dried over Na₂SO₄, filtered, and concentrated under reduced pressure to give a residue and used into the next step without further purification. I-302-6 (4.4 g, crude) was obtained as a white oil. LCMS (Method D): R^{t}=0.421 min, [M+Na]⁺=535.3.

### Step 4: Synthesis of I-302-7

To a solution of I-302-6 (4.4 g, 8.58 mmol, 1 eq) in DCM (10 mL) was added HCl/dioxane (2 M, 20 mL). The mixture was stirred at 25° C. for 1 hr. The reaction mixture was concentrated under reduced pressure to give a residue. The residue was purified by reverse phase column (FA condition). The eluent was concentrated and lyophilized to give a product. I-302-7 (2.09 g, 4.31 mmol, 50.18% yield, 94.489% purity, FA) was obtained as a white solid. LCMS (Method D): R^{t}=0.354 min, [M+H]⁺=413.3. ¹H NMR (400 MHZ, METHANOL-d4) δ=7.80-7.73 (m, 2H), 7.59 (d, J=7.2 Hz, 1H), 7.53 (d, J=7.6 Hz, 1H), 7.40-7.33 (m, 2H), 7.33-7.24 (m, 2H), 4.55 (d, J=5.2 Hz, 1H), 4.43 (d, J=6.0 Hz, 1H), 4.18-4.08 (m, 1H), 4.06-4.01 (m, 1H), 3.96 (s, 1H), 3.87 (s, 1H), 3.61-3.53 (m, 2H), 3.48-3.44 (m, 1H), 3.43-3.38 (m, 1H), 3.27-3.26 (m, 2H), 3.18 (s, 1H), 3.06-3.02 (m, 1H), 3.00-2.96 (m, 1H), 1.22-1.77 (m, 3H) 3.06-3.02 (m, 1H), 3.00-2.96 (m, 1H), 1.22-1.77 (m, 3H).

### Step 5: Synthesis of I-302-9

To a solution of I-302-2 (100 mg, 292.07 μmol, 1 eq) in DMF (1 mL) was added HOAt (39.75 mg, 292.07 μmol, 40.86 μL, 1 eq), EDCI (167.97 mg, 876.20 μmol, 3 eq), NMM (147.71 mg, 1.46 mmol, 160.55 μL, 5 eq) and I-302-3 (133.91 mg, 292.07 μmol, 1 eq, FA salt). The mixture was stirred at 25° C. for 0.5 hr. The mixture was washed with water (3 mL) and extract with EA (2 mL*3). The combined organic layers dried over sodium sulfate, filtered, and concentrated under reduced pressure to give a residue. The residue was purified by reversed phase column (FA condition), the eluent was concentrated under reduced pressure to remove ACN and lyophilized to give I-302-4 (140 mg, 190.00 μmol, 65.05% yield) was obtained as yellow oil. LCMS (Method D): R^{t}=0.577 min, [M+H]⁺=737.3. ¹H NMR (400 MHZ, METHANOL-d4) δ=9.00-8.89 (m, 1H), 8.45-8.34 (m, 1H), 7.73 (d, J=7.6 Hz, 1H), 7.67 (d, J=7.6 Hz, 1H), 7.58 (d, J=7.6 Hz, 1H), 7.49-7.40 (m, 4H), 7.39-7.22 (m, 5H), 4.48 (d, J=5.6 Hz, 1H), 4.37 (d, J=6.0 Hz, 1H), 4.23-4.13 (m, 1H), 4.13 (s, 2H), 3.96 (s, 1H), 4.03 (s, 2H), 3.65-3.52 (m, 4H), 3.52-3.44 (m, 3H), 2.76-2.67 (m, 2H), 1.55 (d, J=4.0 Hz, 9H), 1.31-1.26 (m, 3H), 1.19 (m, 3H).

### Step 6: Synthesis of I-302-5

To a solution of I-302-4 (140 mg, 190.00 μmol, 1 eq) in DCE (1.4 mL) was added Me3SnOH (171.78 mg, 949.99 μmol, 5 eq). The mixture was stirred at 80° C. for 12 hrs. The mixture was washed with aq. KF (10 mL) and diluted with water (5 mL). The mixture was stirred at 25° C. for 1 hr. Then the mixture was adjusted to pH=6˜7 with saturated citric acid aqueous solution. Then the reaction mixture was extracted with DCM (3 mL×3). The combined organic layers were washed with brine (3 mL×3) and dried over sodium sulfate, filtered, and concentrated under reduced pressure to give a residue. The crude product was used into the next step without further purification. I-302-5 (150 mg, crude) was obtained as yellow solid. LCMS (Method D): R^{t}=0.538 min, [M+H]⁺=709.3. ¹H NMR (400 MHZ, METHANOL-d4) δ=8.91 (m, 1H), 8.44-8.31 (m, 1H), 7.70 (m, 2H), 7.58 (d, J=7.6 Hz, 1H), 7.50 (d, J=7.6 Hz, 1H), 7.44-7.26 (m, 7H), 7.23-7.19 (m, 1H), 4.44 (d, J=5.6 Hz, 1H), 4.26 (d, J=5.6 Hz, 1H), 4.20-4.06 (m, 1H), 3.99 (s, 1H), 3.88 (s, 1H), 3.78 (s, 1H), 3.66-3.63 (m, 2H), 3.59 (d, J=5.2 Hz, 1H), 3.53-3.48 (m, 3H), 3.30 (s, 2H), 2.71 (m, 2H), 1.55 (d, J=5.2 Hz, 9H), 1.30-1.25 (m, 3H).

### Step 7: Synthesis of I-302-7

To a solution of I-302-5 (30 mg, 42.33 μmol, 1 eq) in DMF (0.3 mL) was added HOAt (5.76 mg, 42.33 μmol, 5.92 μL, 1 eq), EDCI (24.34 mg, 126.98 μmol, 3 eq), NMM (21.41 mg, 211.63 μmol, 23.27 μL, 5 eq) and I-302-6 (22.58 mg, 42.33 μmol, 1 eq, HCl salt). The mixture was stirred at 25° C. for 1 hr. The mixture was washed with water (1 mL) and extract with EA (2 mL*3). The combined organic layers dried over sodium sulfate, filtered, and concentrated under reduced pressure to give a residue. The residue was purified by reversed phase HPLC (FA condition), the eluent was concentrated under reduced pressure to remove ACN and lyophilized to give I-302-7 (17 mg, 14.09 μmol, 33.29% yield, 98.45% purity) as white solid. LCMS (Method D): R^{t}=0.438 min, [M+H]⁺=1187.3. SFC: R^{t}=0.844 min, 1.042 min.

### Step 8: Synthesis of 1-302

To a solution of I-302-7 (17 mg, 14.31 μmol, 1 eq) in THF (0.2 mL) was added piperidine (0.025 mL). The mixture was stirred at 25° C. for 1 hr. The reaction mixture was concentrated in vacuo. The residue was purified by prep-HPLC (column: CD07-Daisogel SP-100-8-ODS-PK 150*25*10 um; mobile phase: [water (NH4HCO3)-ACN]; gradient: 59%-82% B over 12 min), the eluent was concentrated under reduced pressure to remove ACN and lyophilized to give I-302 (2 mg, 2.04 μmol, 14.22% yield, 98.280% purity) was obtained as white solid. LCMS: R^{t}=0.807 min, [M+H]⁺=965.6. SFC: R^{t}=1.897 min, 2.108 min. 1H NMR (400 MHZ, METHANOL-d4) δ=9.00 (s, 1H), 8.48 (s, 1H), 8.12 (s, 1H), 7.54-7.47 (m, 2H), 7.41 (m, 1H), 7.35-7.26 (m, 5H), 7.11 (s, 1H), 6.60 (s, 1H), 4.99-4.91 (m, 1H), 4.56-4.45 (m, 2H), 3.68 (s, 4H), 3.61 (d, J=8.4 Hz, 6H), 3.40 (s, 2H), 2.92 (s, 2H), 2.73 (m, 2H), 2.37 (s, 2H), 2.33 (d, J=1.6 Hz, 2H), 2.26 (d, J=6.0 Hz, 2H), 2.21-2.10 (m, 2H), 2.02 (s, 1H), 1.95-1.86 (m, 1H), 1.60 (s, 1H), 1.55 (s, 9H), 1.54-1.50 (m, 2H), 1.32-1.25 (m, 4H).

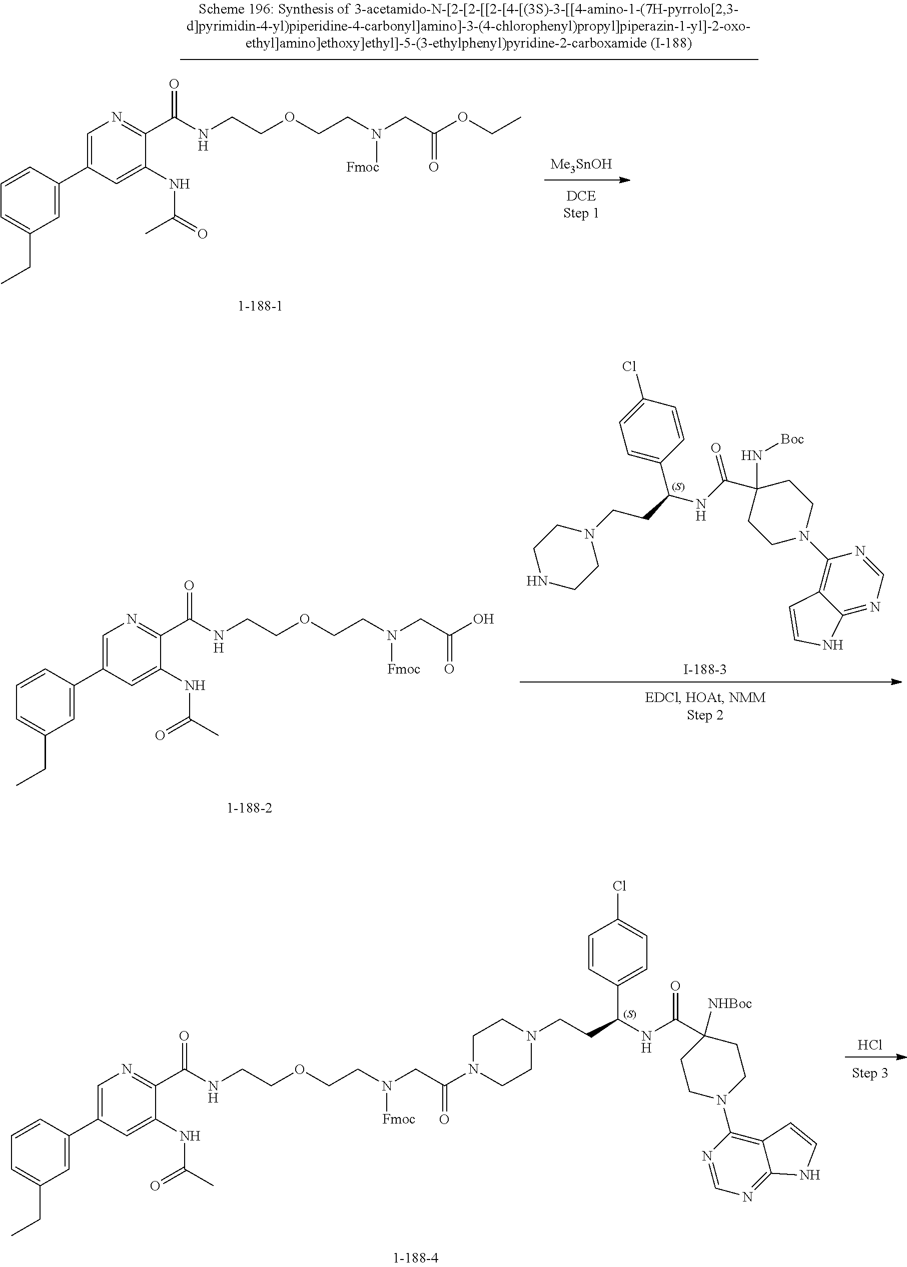

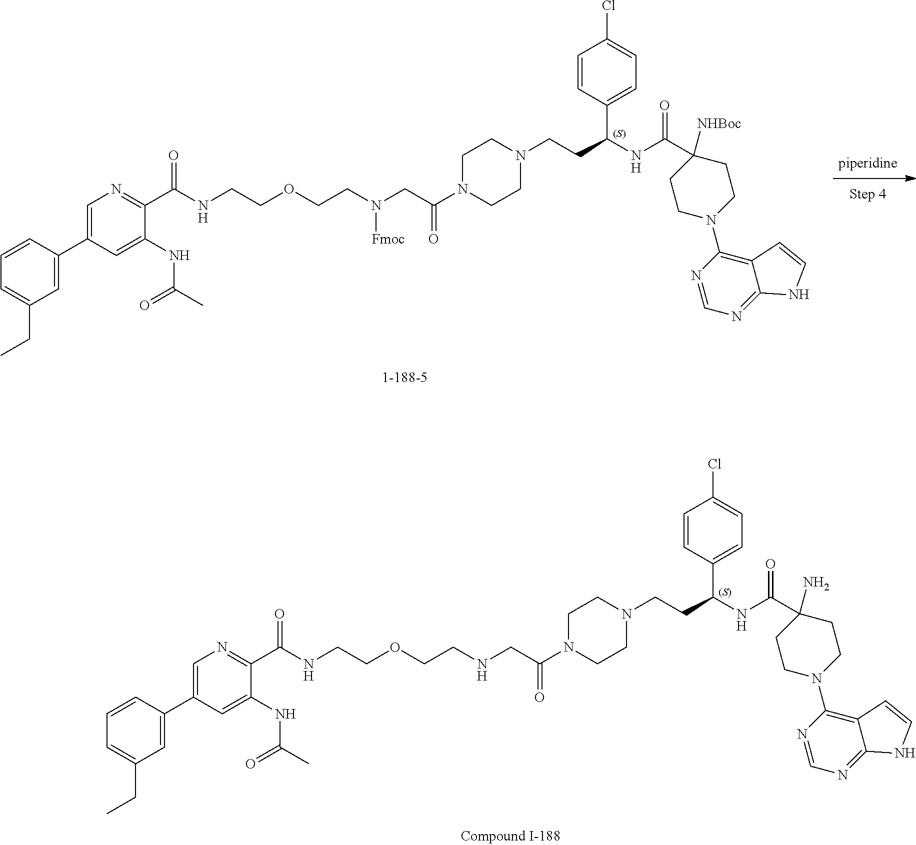

### Step 1: Synthesis of I-188-2.

To a mixture of I-188-1 (80 mg, 117.86 μmol, 1 eq) in DCE (1 mL) was added Me3SnOH (106.56 mg, 589.30 μmol, 5 eq), then the mixture was stirred at 80° C. for 12 hours. The mixture was washed with aq. KF (20 mL) and diluted with water (10 mL). The mixture was stirred at 25° C. for 1 hr. Then the mixture was adjusted to pH=6˜7 with saturated citric acid aqueous solution. Then the reaction mixture was extracted with DCM (10 mL×3). The combined organic layers were washed with brine (15 mL×3) and dried over sodium sulfate, filtered, and concentrated under reduced pressure to give I-188-2 (60 mg, 92.21 μmol, 78.23% yield) as a white solid. LCMS (Method D): R^{t}=0.542 min, (M+H)=651.5.

### Step 2: Synthesis of I-188-4.

To a mixture of I-188-2 (40 mg, 61.47 μmol, 1 eq) and I-188-2 (36.71 mg, 61.47 μmol, 1 eq) in DMF (1 mL) was added HOAt (8.37 mg, 61.47 μmol, 8.60 μL, 1 eq), EDCI (35.35 mg, 184.41 μmol, 3 eq) and NMM (31.09 mg, 307.35 μmol, 33.79 μL, 5 eq), then the mixture was stirred at 20° C. for 1 hour. The reaction mixture was diluted with water 20 mL and extracted with EA 15 mL (5 mL*3). The combined organic layers were concentrated under reduced pressure to give I-188-4 (60 mg, 48.79 μmol, 79.37% yield) as a white solid. LCMS: R^{t}=0.451 min, (M+H)=1229.3.

### Step 3: Synthesis of I-188-5.

To a mixture of I-188-5 (40 mg, 32.52 μmol, 1 eq) in DCM (2 mL) was added HCl/dioxane (2 M, 1 mL), then the mixture was stirred at 20° C. for 1 hour. The reaction mixture was concentrated under reduced pressure to give I-188-5 (40 mg, crude, HCl salt) as a white solid. LCMS (Method D): R^{t}=0.410 min, (M+H)=1129.2.

### Step 4: Synthesis of I-188

A mixture of I-188-5 (40 mg, 35.41 μmol, 1 eq) in piperidine (0.25 mL) and THF (2 mL) was stirred at 20° C. for 1 hour. The reaction mixture was filtered, and concentrated under reduced pressure to give a residue. The crude product was purified by reverse-phase HPLC (0.1% NH₃·H₂O condition). The eluent was concentrated under reduced pressure to remove MeCN and then lyophilized to give I-188 (10.15 mg, 10.90 μmol, 30.78% yield, 97.433% purity) as a white solid. LCMS (Method D): R^{t}=0.325 min, (M+H)=907.3. SFC: R^{t}=0.721 min. ¹H NMR (400 MHZ, METHANOL-d4) δ=9.24 (s, 1H), 8.57-8.53 (m, 1H), 8.11 (s, 1H), 7.53-7.46 (m, 2H), 7.42-7.36 (m, 1H), 7.33-7.27 (m, 5H), 7.11 (d, J=3.6 Hz, 1H), 6.60 (d, J=3.6 Hz, 1H), 4.93 (d, J=6.0 Hz, 1H), 4.53-4.44 (m, 2H), 3.72-3.67 (m, 2H), 3.67-3.62 (m, 5H), 3.56 (s, 2H), 3.47 (s, 2H), 3.44-3.37 (m, 2H), 3.12-3.09 (m, 1H), 2.86-2.80 (m, 2H), 2.76-2.69 (m, 2H), 2.41-2.34 (m, 2H), 2.32-2.28 (m, 2H), 2.25 (s, 3H), 2.20-2.09 (m, 2H), 2.03-1.87 (m, 2H), 1.80-1.68 (m, 2H), 1.62-1.51 (m, 2H), 1.30-1.25 (m, 3H).

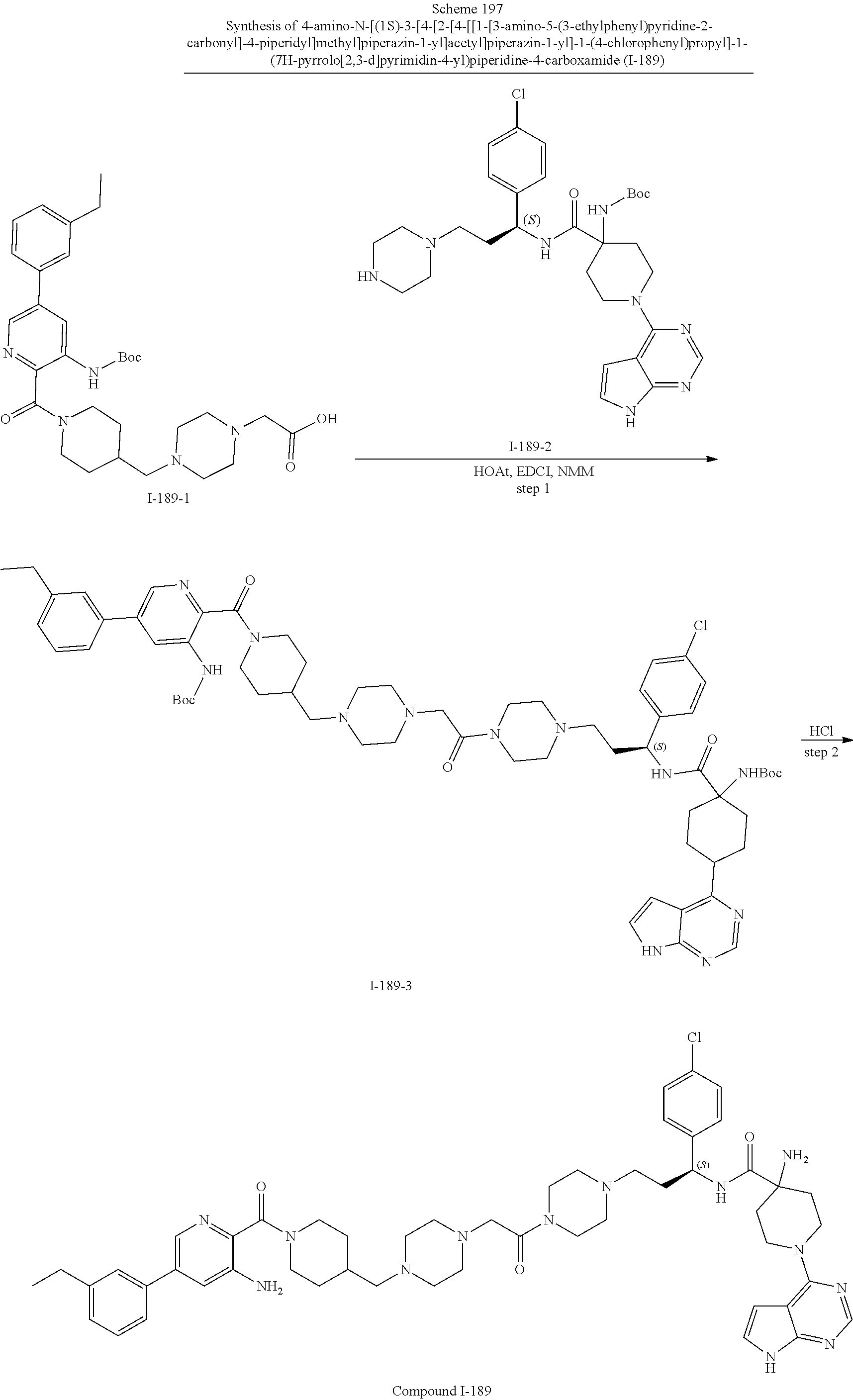

### Step 1: Synthesis of I-189-3

To a solution of I-189-2 (52.78 mg, 88.39 μmol, 1 eq) in DMF (0.5 mL) was added NMM (44.70 mg, 441.93 μmol, 48.59 μL, 5 eq) and EDCI (84.72 mg, 441.93 μmol, 5 eq), HOAt (24.06 mg, 176.77 μmol, 24.73 μL, 2 eq), I-189-1 (50 mg, 88.39 μmol, 1 eq). The mixture was stirred at 25° C. for 1 hr. The reaction mixture was washed with water (0.5 mL) and extracted with DCM (0.5 mL*3), the combined organic phase was dried by Na₂SO₄, concentrated under reduced pressure to give a residue. The residue was purified by reverse-phase (0.1% of FA) and the eluent was lyophilized to give I-189-3 (30 mg, 26.05 μmol, 29.47% yield, 99.393% purity) as a white solid. LCMS (Method D): R^{t}=0.433 min, (M+H)=1145.6.

### Step 2: Synthesis of I-189

To a mixture of I-189-3 (30 mg, 26.20 μmol, 1 eq) in HCl/dioxane (2 M, 0.3 mL), the mixture was stirred at 25° C. for 1 hr. The reaction mixture was filtered, and the filtrate was concentrated under reduced pressure to give a residue. The residue was purified by reverse-phase (0.1% of HCl) and the eluent was lyophilized to give I-189 (4 mg, 4.02 μmol, 15.34% yield, 98.568% purity, HCl) as a white solid. LCMS (Method D): R^{t}=0.288 min, (M+H)=944.4. ¹H NMR (400 MHZ, METHANOL-d4) δ=8.50-8.32 (m, 2H), 8.08-8.07 (m, 1H), 8.08 (d, J=1.6 Hz, 1H), 7.66-7.53 (m, 2H), 7.52-7.40 (m, 5H), 7.37 (d, J=8.4 Hz, 2H), 7.04 (d, J=3.6 Hz, 1H), 5.10-5.04 (m, 1H), 4.78-4.48 (m, 6H), 4.10-3.73 (m, 12H), 3.73-3.61 (m, 3H), 3.44-3.34 (m, 3H), 3.31-3.11 (m, 5H), 2.90-2.72 (m, 4H), 2.55 (d, J=10.0 Hz, 1H), 2.40-2.25 (m, 3H), 2.15-2.05 (m, 3H), 1.55-1.43 (m, 2H), 1.32 (t, J=7.6 Hz, 4H).

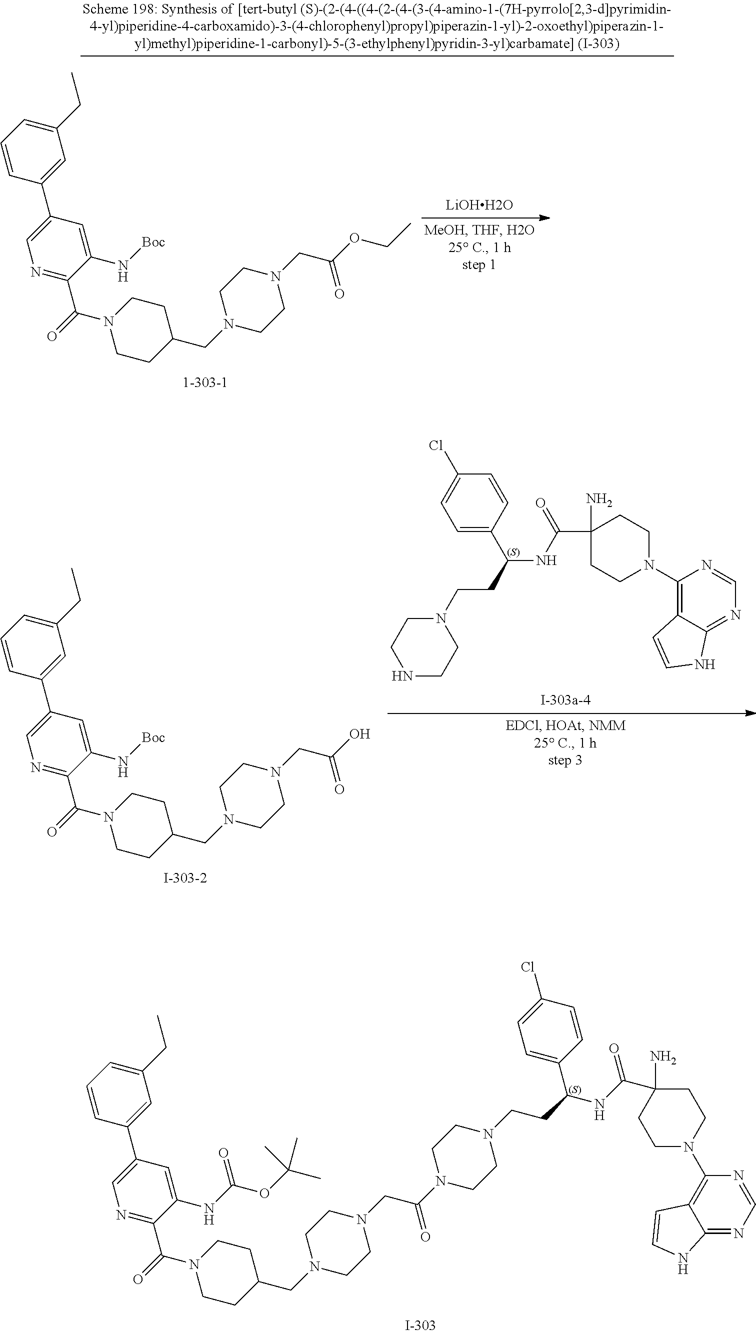

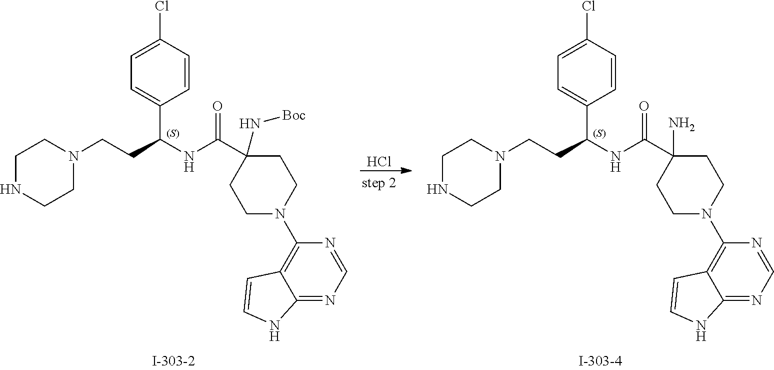

### Step 1: Synthesis of I-303-2

To a solution of I-303-1 (0.2 g, 336.84 μmol, 1 eq) in MeOH (0.6 mL), THF (0.6 mL), and H₂O (0.6 mL) was added LiOH·H₂O (42.40 mg, 1.01 mmol, 3 eq). The mixture was stirred at 25° C. for 0.5 hr. The reaction mixture was concentrated under reduced pressure to give I-303-2 (0.2 g, crude) was obtained as a white solid. LCMS (Method D): R^{t}=0.373 min, (M+H)=566.3.

### Step 2: Synthesis of I-303-4

A solution of I-303-2 (50 mg, 83.73 μmol, 1 eq) in HCl/dioxane (0.5 mL). The mixture was stirred at 25° C. for 0.5 hr. The reaction mixture was concentrated under reduced pressure to give I-303-4 (50 mg, crude, HCl) d as a red solid. LCMS (Method D): R^{t}=0.141 min, (M+H)=497.3.

### Step 3: Synthesis of I-303

To a solution of I-303-2 (53.02 mg, 93.72 μmol, 1 eq), I-303-4 (50 mg, 93.72 μmol, 1 eq, HCl) in DMF (1 mL) was added EDCI (53.90 mg, 281.16 μmol, 3 eq) and NMM (47.40 mg, 468.61 μmol, 51.52 μL, 5 eq), HOAt (12.76 mg, 93.72 μmol, 13.11 μL, 1 eq). The mixture was stirred at 25° C. for 0.5 hr. The residue was purified by prep-HPLC (column: CD07-Daisogel SP-100-8-ODS-PK 150*25*10 um; mobile phase: [water (NH4HCO3)-ACN]; gradient: 50%-80% B over 10 min) the eluent was concentrated to remove ACN and lyophilized to get I-303 (8.35 mg, 7.70 μmol, 8.22% yield, 96.352% purity) as a yellow solid. LCMS: R^{t}=0.345 min, (M+H)=1044.5. ¹H NMR (400 MHZ, DMSO-d₆) δ=11.64 (s, 1H), 9.04 (s, 1H), 8.77-8.67 (m, 1H), 8.57 (d, J=2.0 Hz, 1H), 8.26 (d, J=1.6 Hz, 1H), 8.11 (s, 1H), 7.58-7.48 (m, 2H), 7.45-7.42 (m, 1H), 7.38-7.27 (m, 5H), 7.17-7.11 (m, 1H), 6.57 (d, J=1.6 Hz, 1H), 4.91-4.80 (m, 1H), 4.53-4.31 (m, 3H), 3.76-3.66 (m, 1H), 3.58-3.50 (m, 3H), 3.45 (s, 2H), 3.09 (s, 2H), 3.05-2.96 (m, 2H), 2.81-2.65 (m, 4H), 2.37 (d, J=2.4 Hz, 8H), 2.21 (d, J=6.4 Hz, 3H), 2.09 (d, J=18.8 Hz, 3H), 2.05-1.70 (m, 8H), 1.69-1.52 (m, 2H), 1.48-1.39 (m, 11H), 1.25-1.21 (m, 5H)

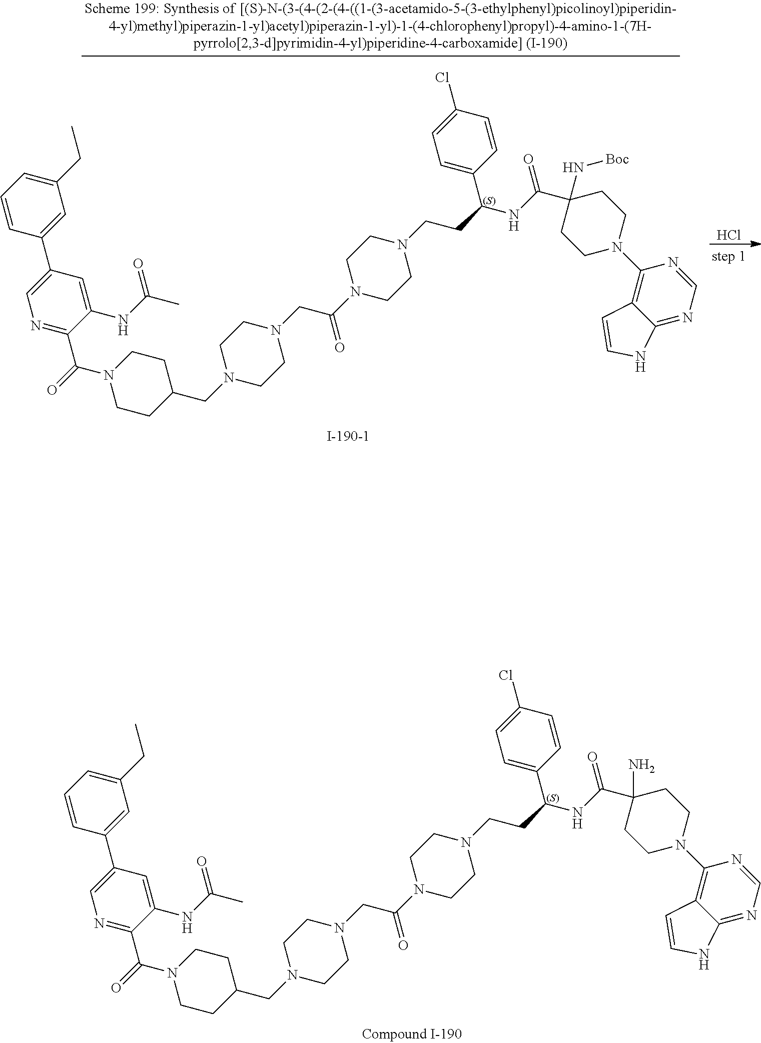

### Step 1: Synthesis of I-190

A solution of I-190-1 (35 mg, 32.21 μmol, 1 eq) in HCl/dioxane (0.5 mL). The mixture was stirred at 25° C. for 0.5 hr. LC-MS( ). The reaction mixture was concentrated under reduced pressure to give I-190 (25.82 mg, 24.68 μmol, 76.63% yield, 97.794% purity, HCl) as a yellow solid. LCMS: R^{t}=0.277 min, (M+H)=986.5. ¹H NMR (400 MHZ, METHANOL-d4) δ=8.76 (s, 1H), 8.47 (d, J=1.6 Hz, 1H), 8.40 (s, 1H), 7.61-7.51 (m, 2H), 7.49-7.40 (m, 4H), 7.35 (d, J=8.4 Hz, 3H), 7.02 (d, J=3.6 Hz, 1H), 5.08-5.04 (m, 1H), 4.75 (d, J=2. Hz, 3H), 4.68-4.49 (m, 5H), 4.06-3.82 (m, 9H), 3.81-3.57 (m, 6H), 3.40-3.33 (m, 2H), 3.24 (d, J=10.8 Hz, 4H), 3.06-2.93 (m, 1H), 2.90-2.80 (m, 1H), 2.79-2.68 (m, 3H), 2.59-2.46 (m, 1H), 2.38-2.23 (m, 3H), 2.20 (s, 3H), 2.13-2.03 (m, 2H), 1.92 (d, J=11.2 Hz, 1H), 1.63-1.42 (m, 2H), 1.32-1.28 (m, 3H).

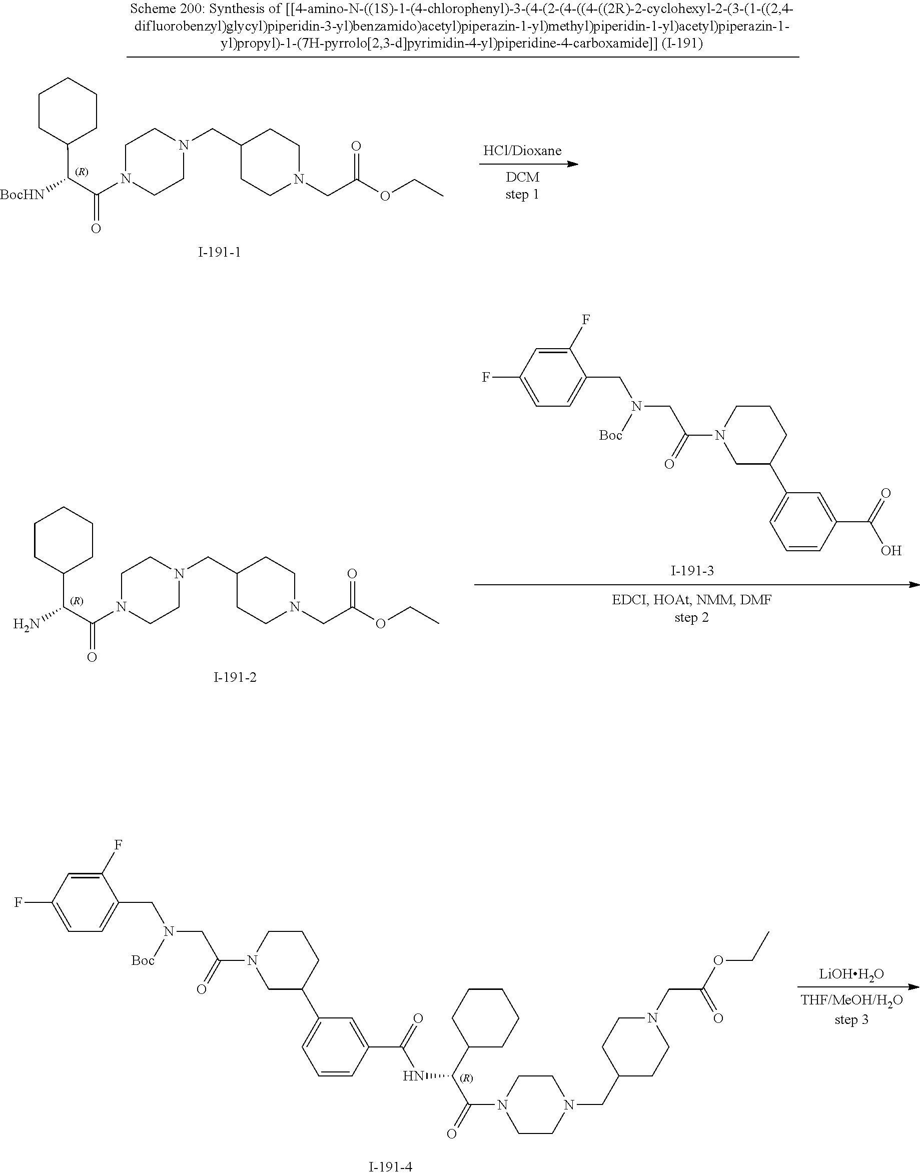

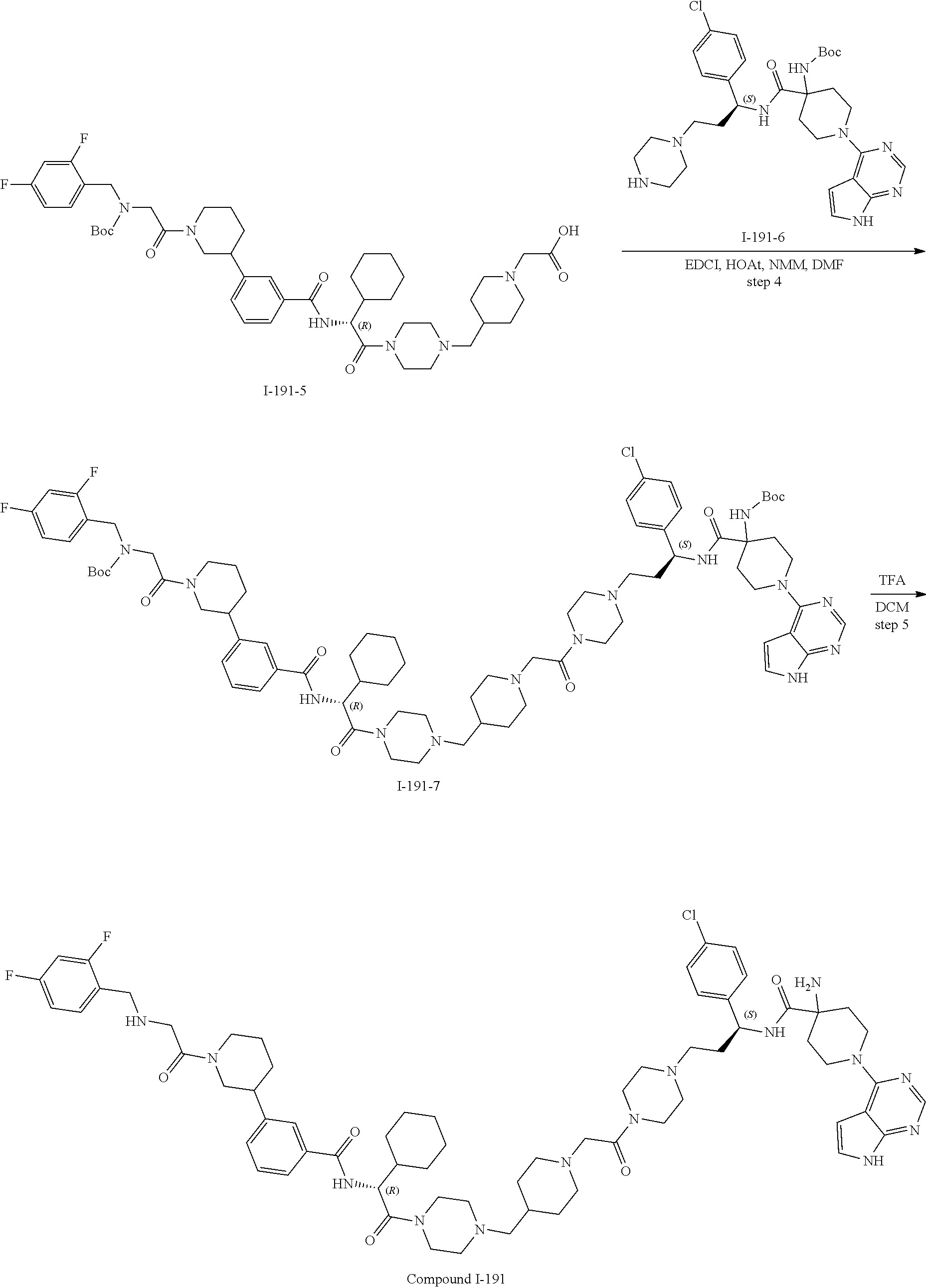

### Step 1: Synthesis of I-191-2

To a solution of I-191-1 (1 g, 1.97 mmol, 1 cg) in DCM (10 mL) was added HCV/dioxane (10 mL). The mixture was stirred at 25° C. for 0.5 hr. LC-MS( ). The reaction mixture was concentrated under reduced pressure to give a residue. I-191-2 (1 g, crude, HCl) was obtained as a white solid. LCMS:.

### Stop 2; Synthesis of I-191-4

To a solution of I-191-2 (1 g. 2.25 mmol, 1 eq, HCl), I-191-3 (1.10 g. 2.25 mmol, 1 eq) in DMF (10 mL) was added NMM (1.14 g, 11.24 mmol, 1.24 mL, 5 eq), EDCI (1.29 g, 6.74 mmol, 3 eq) and HOAt (305.84 mg, 2.25 mmol, 314.33 μL, 1 eq). The mixture was stirred at 25° C. for 1 hr. The reaction mixture was diluted with H₂O (30 mL) and extracted with EtOAc (20 mL*3). The combined organic phase was dried over anhydrous Na₂SO₄, filtered, and concentrated under reduced pressure to give a residue. The residue was purified by reversed phase column (0.1% FA), then the eluent was concentrated to remove ACN and lyophilized to get I-191-4 (0.9 g. 983.28 μmol, 43.76% yield, 96.043% purity), which was a white solid. LCMS: R^{t}=0.430 min, [M+H]⁺=879.6.049. SFC: R^{t}=1.947 min/2.097 min.

### Step 3: Synthesis of I-191-5

To a solution of I-191-4 (0.9 g, 1.02 mmol, 1 eq) in THF (3 mL), MeOH (3 mL) and H₂O (3 mL) was added LiOH·H₂O (128.89 mg, 3.07 mmol, 3 eq). The mixture was stirred at 25° C. for 0.5 hr. The reaction mixture was adjusted pH=6 by HCl (1M), and then extracted with DCM 30 mL (10 mL*3). The combined organic layers were dried over Na₂SO₄, filtered, and concentrated under reduced pressure to give I-191-5 (0.8 g, 940.04 μmol, 91.82% yield), which was obtained as a white solid. LCMS (Method D): R^{t}=0.425 min, [M+H]⁺=851.4. SFC: R^{t}=1.424 min/1.518 min.

### Step 4: Synthesis of I-191-7

To a solution of I-191-5 (300.00 mg, 352.51 μmol, 1 eq), I-191-6 (210.50 mg, 352.51 μmol, 1 eq) in DMF (3 mL) was added EDCI (202.73 mg, 1.06 mmol, 3 eq) and HOAt (47.98 mg, 352.51 μmol, 49.31 μL, 1 eq), NMM (178.28 mg, 1.76 mmol, 193.78 μL, 5 eq). The mixture was stirred at 25° C. for 0.5 hr. The reaction mixture was purified by reversed phase column (0.1% TFA) the eluent was concentrated to remove ACN and lyophilized to get I-191-7 (0.4 g, 279.69 μmol, 79.34% yield) as a white solid. LCMS: R^{t}=0.362 min, [M+H]⁺=1431.0.675. SFC: R^{t}=4.404 min/4.510 min.

### Step 5: Synthesis of I-191

A mixture of I-191-7 (0.4 g, 259.04 μmol, 1 eq, TFA) and TFA (1.23 g, 10.77 mmol, 800.00 μL, 41.58 eq) in DCM (4 mL) was stirred at 25° C. for 0.5 hr. The reaction mixture was concentrated under reduced pressure to give a residue. The residue was purified by reversed phase column (0.1% NH₃·H₂O). The eluent was concentrated to remove ACN and lyophilized to get I-191 (101.85 mg, 81.30 μmol, 31.39% yield, 98.179% purity), which was obtained as a white solid. LCMS: R^{t}=2.510 min, [M+H]⁺=1229.8. SFC: R^{t}=4.285 min/6.092 min. ¹H NMR (400 MHZ, METHANOL-d4) δ=8.12 (s, 1H), 7.79-7.66 (m, 2H), 7.52-7.39 (m, 3H), 7.38-7.26 (m, 4H), 7.12 (d, J=3.6 Hz, 1H), 7.00-6.88 (m, 2H), 6.62 (d, J=3.6 Hz, 1H), 4.99-4.96 (m, 1H), 4.89 (d, J=5.6 Hz, 2H), 4.62-4.43 (m, 4H), 3.89-3.77 (m, 4H), 3.76-3.59 (m, 8H), 3.57-3.51 (m, 2H), 3.49-3.40 (m, 1H), 3.19 (s, 2H), 3.15-3.07 (m, 1H), 2.90 (d, J=11.6 Hz, 2H), 2.83-2.69 (m, 2H), 2.52-2.33 (m, 9H), 2.21 (d, J=6.4 Hz, 2H), 2.16-2.01 (m, 5H), 2.00-1.82 (m, 5H), 1.77 (d, J=10.8 Hz, 4H), 1.72-1.66 (m, 2H), 1.64-1.50 (m, 4H), 1.34-1.18 (m, 5H), 1.16-1.05 (m, 2H). ¹⁹F NMR (400 MHZ, METHANOL-d4) δ=−113.348, −116.252.

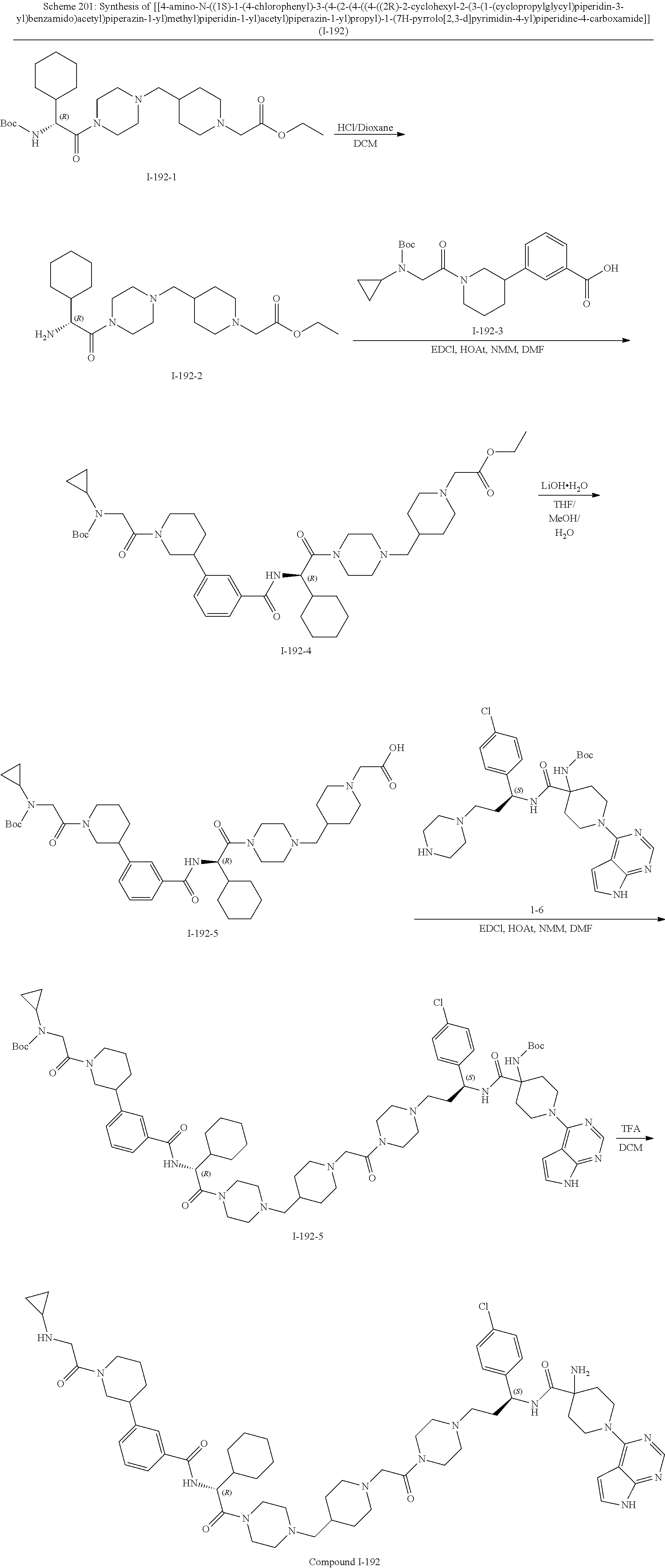

### Step 1; Synthesis of I-192-2

To a solution of I-192-1 (1.3 g, 2.56 mmol, 1 eq) in DCM (6 mL) was added HCl/dioxane (2 M, 12 mL, 9.39 eq). The mixture was stirred at 25° C. for 1 hr. showed I-192-1 was consumed completely. The reaction mixture was concentrated under reduced pressure to give I-192-2 (1.5 g, crude, HCl) as a white solid which was used for next step directly. LCMS (Method D): R^{t}=0.0516 min, [M+H]⁺=409.3.

### Step 2: Synthesis of I-192-4

To a solution of I-192-2 (900 mg, 2.02 mmol, 1 eq, HCl), I-192-3 (813.94 mg, 2.02 mmol, 1 eq) in DMF (9 mL) was added EDCI (775.35 mg, 4.04 mmol, 2 eq) and HOAt (275.26 mg, 2.02 mmol, 282.90 μL, 1 eq) and NMM (1.02 g, 10.11 mmol, 1.11 mL, 5 eq). The mixture was stirred at 25° C. for 1 hr. The reaction mixture was diluted with H₂O (10 mL) and then extracted with DCM (10 mL*3), the combined organic phase was dried by Na₂SO₄, filtered, and concentrated. The crude product was purified by reversed phase column (0.1% FA condition), the eluent was concentrated to remove ACN and lyophilized to get I-192-4 (720 mg, 907.89 μmol, 44.89% yield) as a white solid. LCMS (Method D): R^{t}=0.339 min, [M+H]⁺=793.5. SFC: R^{t}=3.694, 3.915 min.

### Step 3: Synthesis of I-192-5

To a solution of I-192-4 (600 mg, 756.58 μmol, 1 eq) in THF (2 mL), MeOH (2 mL) and H₂O (2 mL) was added LiOH·H₂O (63.50 mg, 1.51 mmol, 2 eq). The mixture was stirred at 25° C. for 1 hr. The aqueous phase was adjusted to pH=6 with 1N HCl, then diluted with H₂O (5 mL) and then extracted with DCM (10 mL*3). The combined organic phase was dried by Na₂SO₄, filtered, and concentrated. The product was used for next step. I-192-5 (630 mg, crude) was obtained as a white solid. LCMS (Method D): R^{t}=0.350 min, [M+H]⁺=765.5. SFC: R^{t}=1.746 min.

### Step 4: Synthesis of I-192-7

To a solution of I-192-5 (300 mg, 392.16 μmol, 1 eq), I-192-6 (187.34 mg, 313.73 μmol, 0.8 eq) in DMF (3 mL) was added EDCI (150.36 mg, 784.32 μmol, 2 eq) and HOAt (53.38 mg, 392.16 μmol, 54.86 μL, 1 eq) and NMM (198.33 mg, 1.96 mmol, 215.58 μL, 5 eq). The mixture was stirred at 25° C. for 1 hr. The reaction mixture was added H₂O (5 mL) and then extracted with DCM (10 mL*3), the combined organic phase was washed with dried by Na₂SO₄, filtered, and concentrated. The crude product was purified by reversed phase column (0.1% TFA condition), the eluent was concentrated to remove ACN and lyophilized to get product. I-192-7 (170 mg, 126.48 μmol, 32.25% yield) was obtained as a white solid. LCMS (Method D): R^{t}=0.326 min, [M+H]⁺=1343.8.

### Step 5: Synthesis of I-192

To a solution of I-192-7 (170 mg, 126.48 μmol, 1 eq) in DCM (1.7 mL) was added TFA (782.85 mg, 6.87 mmol, 510.00 μL, 54.28 eq). The mixture was stirred at 25° C. for 1 hr. The reaction mixture was concentrated under reduced pressure to give a residue. The crude product was purified by reversed phase column (0.1% NH₃·H₂O condition), the eluent was concentrated to remove ACN and lyophilized to get product. I-192 (118.37 mg, 102.99 μmol, 81.43% yield, 99.522% purity) was obtained as a white solid. LCMS: R^{t}=2.297 min, [M+H]⁺=1143.9. SFC: R^{t}=1.399 min. ¹H NMR (400 MHZ, METHANOL-d4) δ=8.12 (s, 1H), 7.76 (s, 1H), 7.73-7.67 (m, 1H), 7.55-7.39 (m, 2H), 7.33 (s, 3H), 7.12 (d, J=3.6 Hz, 1H), 6.62 (d, J=3.6 Hz, 1H), 4.98 (t, J=6.8 Hz, 1H), 4.92-4.87 (m, 2H), 4.61-4.46 (m, 3H), 3.94-3.80 (m, 2H), 3.77-3.52 (m, 11H), 3.22-3.10 (m, 3H), 2.93-2.70 (m, 4H), 2.54-2.31 (m, 10H), 2.26-2.17 (m, 4H), 2.11-2.02 (m, 4H), 1.94-1.83 (m, 4H), 1.76 (d, J=9.6 Hz, 4H), 1.72-1.53 (m, 6H), 1.37-1.18 (m, 6H), 1.17-1.05 (m, 2H), 0.59-0.31 (m, 4H).

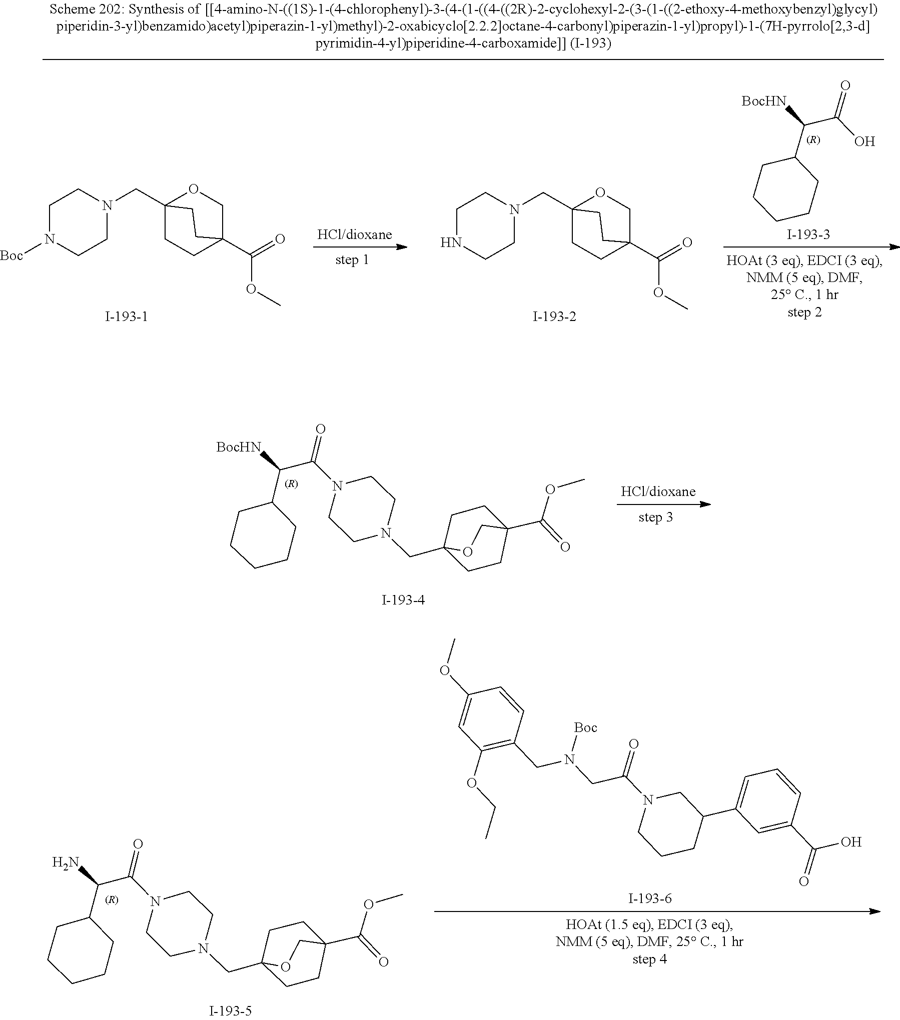

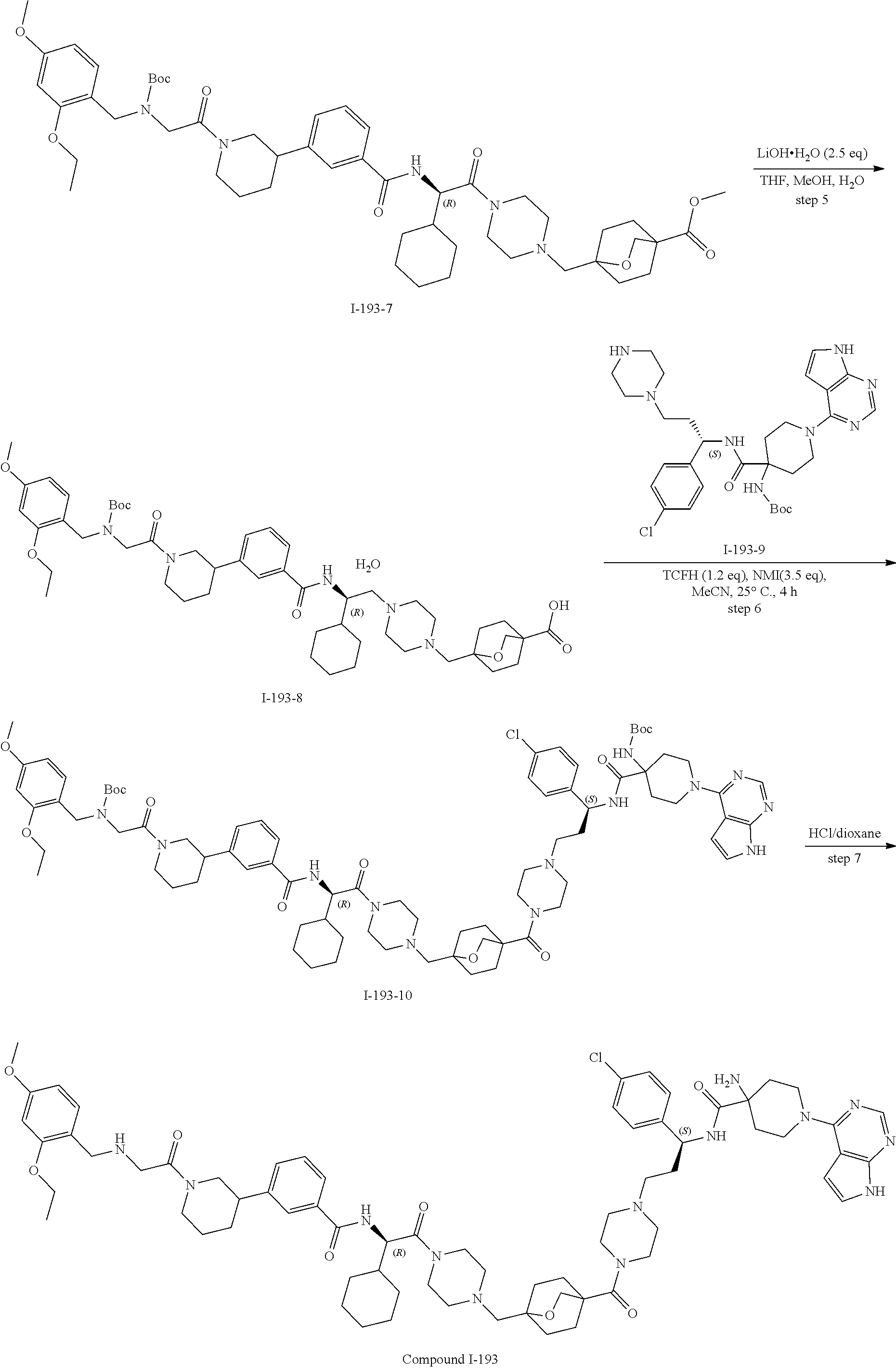

### Step 1: Synthesis of I-193-2

A solution I-193-1 (1 g, 2.71 mmol, 1 eq) and HCl/dioxane (10 mL) was stirred at 25° C. for 1 h. The reacting mixture was concentrated to give crude product. The crude product was used into next step directly. I-193-2 (1.1 g, crude, HCl salt) was obtained as a white solid. LCMS: R^{t}=0.261 min, [M+H]⁺=269.1. ¹H NMR (400 MHZ, DMSO-d₆) δ=11.49-10.63 (m, 1H), 9.80 (br s, 2H), 3.86 (s, 2H), 3.60 (s, 4H), 3.57 (s, 6H), 3.23 (s, 2H), 2.16-1.97 (m, 2H), 1.88 (t, J=7.6 Hz, 4H), 1.73-1.60 (m, 2H).

### Step 2: Synthesis of I-193-4

To a solution of I-193-3 (850 mg, 3.30 mmol, 1 eq) in DMF (10 mL) was added EDCI (1.90 g, 9.91 mmol, 3 eq), HOAt (1.35 g, 9.91 mmol, 1.39 mL, 3 eq), NMM (1.67 g, 16.52 mmol, 1.82 mL, 5 eq) and I-193-2 (1.01 g, 3.30 mmol, 1 eq, HCl). Then the reaction mixture was stirred at 25° C. for 1 h. The reaction mixture was quenched with water (20 mL) and extracted with EA (3*30 mL). The combined organic phase was washed by sat. Solution of NaCl (20 mL), dried by Na₂SO₄, filtered, and concentrated. The crude product was purified by reverse phase column (80 g of XB—C18, 20-40 μm, 120 Å) Mobile phase: A for H₂O (0.1% FA, v/v) and B for acetonitrile; Gradient: B5%-100% in 30 min: Flow rate: 40 ml/min; Column temperature: R.T. Wavelength: 220 nm/254 nm). After purification, the eluent was concentrated to remove MeCN and lyophilized to give product. I-193-4 (1.1 g, 2.17 mmol, 65.60% yield) was obtained as a white solid. LCMS: R^{t}=0.433 min, [M+H]⁺=508.4.

### Step 3: Synthesis of I-193-5

A solution of I-193-4 (1.1 g, 2.17 mmol, 1 eq) HCl/dioxane (11 mL) was stirred at 25° C. for 1 h. The reaction mixture was concentrated to give the crude product. The crude product was used into next step directly. I-193-5 (1.2 g, crude, HCl salt) was obtained as yellow gum. LCMS: R^{t}=0.359 min, [M+H]⁺=408.4.

### Step 4: Synthesis of I-193-7

To a solution of I-193-6 (1.1 g, 2.09 mmol, 1 eq) and I-193-5 (1.02 g, 2.30 mmol, 1.1 eq, HCl) in DMF (11 mL) was added EDCI (1.20 g, 6.27 mmol, 3 eq), HOAt (426.46 mg, 3.13 mmol, 438.29 μL, 1.5 eq), NMM (1.06 g, 10.44 mmol, 1.15 mL, 5 eq). Then the reaction mixture was stirred at 25° C. for 1 h. The reaction mixture was quenched with water (15 mL), extracted with EA (15 mL*3). The combined organic phase was washed by a saturated solution of NaCl (20 mL), dried by anhydrous Na₂SO₄, filtered, and the filtrates was concentrated to give crude product. The crude product was purified by reverse phase column (80 g of XB—C18, 20-40 μm, 120 Å) Mobile phase: A for H₂O (0.1% FA v/v) and B for acetonitrile; Gradient: B 5%-100% in 15 min: Flow rate: 50 ml/min; Column temperature: R.T. Wavelength: 220 nm/254 nm). After purification, the eluent was concentrated to remove MeCN and lyophilized to give product. I-193-7 (1.3 g, 1.42 mmol, 67.93% yield, N/A purity) was obtained as a white solid. LCMS: R^{t}=0.538 min, [M+H]⁺=916.6.

### Step 5: Synthesis of I-193-8

To a solution of I-193-7 (1.3 g, 1.42 mmol, 1 eq) in MeOH (4 mL), THF (4 mL) and H₂O (4 mL) was added LiOH·H₂O (148.85 mg, 3.55 mmol, 2.5 eq). Then the reaction mixture was stirred at 25° C. for 3 h. The reaction mixture was concentrated to give residue, the residue was diluted with water (5 mL) and then adjust to a pH=6 by citric acid and then extracted with EA (5 mL*3). The combined organic phase was dried by anhydrous Na₂SO₄, filtered to give filtrates concentrated to give product. The crude product was used into next step directly. I-193-8 (1.2 g, 1.33 mmol, 93.74% yield, N/A purity) was obtained as a white solid. LCMS: R^{t}=0.537 min, [M+H]⁺=902.5. SFC: R^{t}=3.849 min, 4.588 min. _H1.

### Step 6: Synthesis of I-193-10

To a solution of I-193-8 (500 mg, 554.25 μmol, 1 eq) and I-193-9 (330.97 mg, 554.25 μmol, 1 eq) in MeCN (5 mL) was added NMI (159.27 mg, 1.94 mmol, 154.63 μL, 3.5 eq). The reaction mixture was stirred 5 min until it is clear, then TCFH (186.61 mg, 665.10 μmol, 1.2 eq) was added to the reaction mixture and then stirred at 25° C. for 4 h, and 24% of I-193-8 remained. The reaction mixture was quenched with water (20 mL) and extracted with EA (10 mL*3). The combined organic phase was dried by anhydrous Na₂SO₄, filtered to give filtrates concentrated to give product. The crude was purified by prep-HPLC (column: Phenomenex luna C18 150*40 mm*15 um; mobile phase: [water (FA)-ACN]; gradient: 22%-52% B over 15 min), after purification, then the eluents was concentrated to remove organic solvents and lyophilized to give product. I-193-10 (200 mg, 135.02 μmol, 24.36% yield, N/A purity) was obtained as a yellow solid. LCMS: R^{t}=0.520 min, [M+H]⁺=1480.9.

### Step 7: Synthesis of I-193

To a solution of I-193-10 (200 mg, 135.02 μmol, 1 eq) in dioxane (1 mL) was added HCl/dioxane (1 mL) at 0° C., the reaction mixture was stirred at 0-10° C. for 1 h. The reaction mixture was quenched with a saturated solution of NaHCO₃ (20 mL) slowly at 0° C., and extracted with EA:MeOH=2:1 (5 mL*3). The combined organic phase was dried by anhydrous Na₂SO₄, filtered, and concentrated to give product. The crude was purified by prep-HPLC (column: Phenomenex luna C18 150*25 mm*10 um; mobile phase: [water (FA)-ACN]; gradient: 8%-38% B over 9 min). After purification, then the eluent was concentrated to remove organic solvents and lyophilized to give product. I-193 (130 mg, 100.99 μmol, 74.80% yield, 99.516% purity) was obtained as a white solid. LCMS: R^{t}=0.786 min, [M+H]⁺=1281.0. HPLC: R^{t}=3.629 min. SFC: R^{t}=4.397 min, 5.181 min. _H32. ¹H NMR (400 MHZ, DMSO-d₆) δ=11.65 (s, 1H), 8.75 (d, J=8.0 Hz, 1H), 8.47 (t, J=9.2 Hz, 1H), 8.11 (s, 1H), 7.83-7.70 (m, 2H), 7.45-7.37 (m, 2H), 7.37-7.30 (m, 4H), 7.19-7.12 (m, 2H), 6.57 (d, J=3.6 Hz, 1H), 6.52-6.40 (m, 2H), 4.93-4.68 (m, 2H), 4.50-4.32 (m, 3H), 4.06-3.93 (m, 2H), 3.78 (br s, 2H), 3.72 (d, J=6.4 Hz, 3H), 3.63-3.49 (m, 10H), 3.46-3.35 (m, 3H), 3.13-2.95 (m, 1H), 2.75-2.57 (m, 2H), 2.45 (br s, 1H), 2.39 (d, J=1.8 Hz, 2H), 2.35-2.30 (m, 2H), 2.20 (d, J=7.2 Hz, 9H), 2.10-2.03 (m, 2H), 1.97-1.82 (m, 7H), 1.80-1.65 (m, 8H), 1.63-1.53 (m, 4H), 1.42 (t. J=14.0 Hz, 3H), 1.35-1.27 (m, 3H), 1.22-1.09 (m, 3H), 0.99 (d, J=11.6 Hz, 2H).

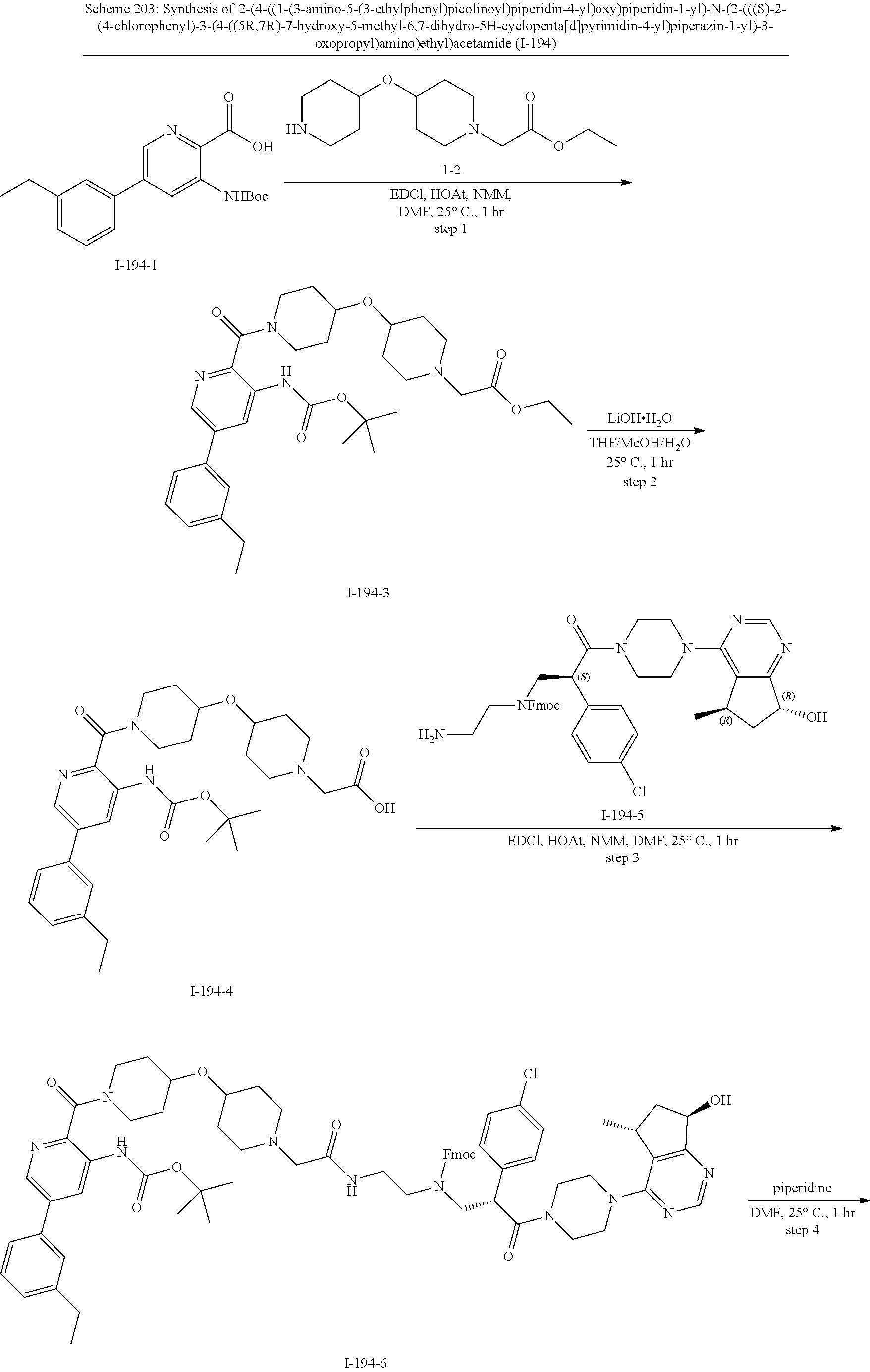

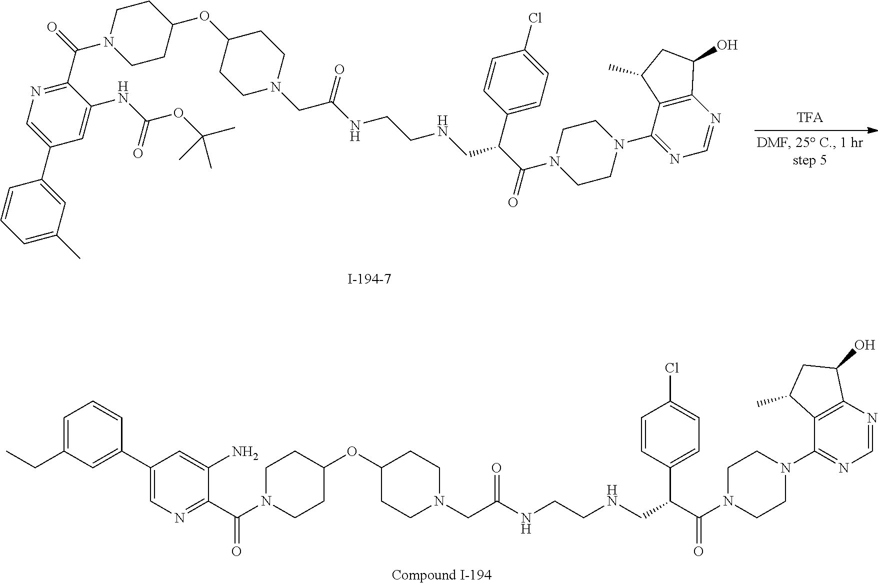 [SMILES: Cc1cccc(-c2cnc(C(=O)N3CCC(OC4CCN(CC(=O)NCCNC[C@@H](C(=O)N5CCN(c6ncnc7c6[C@H](C)C[C@H]7O)CC5)c5ccc(Cl)cc5)CC4)CC3)c(NC(=O)OC(C)(C)C)c2)c1 ; CCc1cccc(-c2cnc(C(=O)N3CCC(OC4CCN(CC(=O)NCCNC[C@@H](C(=O)N5CCN(c6ncnc7c6[C@H](C)C[C@H]7O)CC5)c5ccc(Cl)cc5)CC4)CC3)c(N)c2)c1]

### Step 1: Synthesis of I-194-3

To a solution of I-194-1 (500 mg, 1.46 mmol, 1 eq) in DMF (5 mL) was added HOAt (198.77 mg, 1.46 mmol, 204.28 μL, 1 eq), EDCI (839.84 mg, 4.38 mmol, 3 eq), NMM (738.54 mg, 7.30 mmol, 802.76 μL, 5 eq) and I-194-2 (394.83 mg, 1.46 mmol, 1 eq). The mixture was stirred at 25° C. for 1 hr. The mixture was washed with water (5 mL) and extract with EA (2 mL+3). The combined organic layers dried over sodium sulfate, filtered, and concentrated under reduced pressure to give a residue. The residue was purified by flash silica gel chromatography (ISCO®; 40 g SepaFlash® Silica Flash Column, Eluent of 0˜70%

EA/MeOH gradient @ 60 mL/min) and then the eluent was concentrated in vacuo to give I-194-3 (584 mg, 969.77 μmol, 66.41% yield, 98.760% purity) was obtained as yellow oil. LCMS (Method D): R^{t}=0.423 min, [M+H]⁺=595.5_2000. ¹H NMR (400 MHZ, METHANOL-d4) δ=8.52 (d, J=2.0 Hz, 1H), 8.42 (d, J=2.0 Hz, 1H), 7.54-7.45 (m, 2H), 7.41 (m, 1H), 7.29 (d, J=7.6 Hz, 1H), 4.17 (m, 2H), 4.11-4.05 (m, 2H), 3.78 (m, 1H), 3.73-3.63 (m, 1H), 3.60-3.50 (m, 2H), 3.20 (s, 2H), 2.81 (d, J=4.4 Hz, 2H), 2.74 (m, 2H), 2.38 (m, 2H), 1.97-1.84 (m, 4H), 1.70-1.60 (m, 4H), 1.53 (s, 9H), 1.31-1.27 (m, 3H), 1.25-1.19 (m, 3H).

### Step 2: Synthesis of I-194-4

To a solution of I-194-3 (150 mg, 252.21 μmol, 1 eq) in THF (0.5 mL), H₂O (0.5 mL) and MeOH (0.5 mL) was added LiOH·H₂O (31.75 mg, 756.63 μmol, 3 eq). The mixture was stirred at 25° C. for 1 hr. The pH was acidified to 3˜4 with 1N HCl, and the mixture was washed with water (2 mL) and extract with EA (3 mL*3). The combined organic layers dried over sodium sulfate, filtered, and concentrated under reduced pressure to give a residue. The crude product was used into the next step without further purification. I-194-4 (164 mg, crude) was obtained as yellow solid. LCMS (Method D): R^{t}=0.424 min, [M+H]⁺=567.4. ¹H NMR (400 MHZ, CHLOROFORM-d) δ=8.89 (s, 1H), 8.63 (s, 1H), 8.47 (s, 1H), 7.45 (s, 2H), 7.42-7.37 (m, 1H), 4.19-4.08 (m, 1H), 3.99 (s, 2H), 3.89 (s, 1H), 3.77-3.60 (m, 4H), 3.48-3.39 (m, 2H), 2.76-2.71 (m, 2H), 2.46 (s, 1H), 2.22-2.10 (m, 2H), 2.05 (s, 1H), 2.03-1.86 (m, 4H), 1.72 (m, 2H), 1.53 (s, 9H), 1.31-1.26 (m, 5H).

### Step 3: Synthesis of I-194-6

To a solution of I-194-4 (94.75 mg, 167.20 μmol, 1.2 eq) in DMF (1 mL) was added HOAt (18.97 mg, 139.34 μmol, 19.49 μL, 1 eq), EDCI (80.13 mg, 418.01 μmol, 3 eq), NMM (70.47 mg, 696.69 μmol, 76.60 μL, 5 eq) and I-194-5 (100 mg, 139.34 μmol, 1 eq, HCl). The mixture was stirred at 25° C. for 1 hr. The mixture was washed with water (1 mL) and extract with EA (2 mL*3). The combined organic layers dried over sodium sulfate, filtered, and concentrated under reduced pressure to give a residue. The residue was purified by reversed phase HPLC (FA condition), the eluent was concentrated under reduced pressure to remove ACN and lyophilized to give I-194-6 (74 mg, 48.13 μmol, 34.55% yield, 80% purity) was obtained as white solid. LCMS (Method D): R^{t}=0.453 min, [M/2+H]⁺=615.7.

### Step 4: Synthesis of I-194-7

To a solution of I-194-6 (64 mg, 52.04 μmol, 1 eq) in THF (0.6 mL) was added piperidine (0.08 mL). The mixture was stirred at 25° C. for 1 hr. The mixture was washed with water (1 mL) and extract with EA (2 mL*3). The combined organic layers dried over sodium sulfate, filtered, and concentrated under reduced pressure to give a residue. The residue was purified by reversed phase HPLC (FA condition), the eluent was concentrated under reduced pressure to remove ACN and lyophilized to give I-194-7 (52 mg, 50.71 μmol, 97.45% yield, 98.265% purity) was obtained as a white solid. LCMS (Method D): R^{t}=0.380 min, [M+H]⁺=1008.5. SFC: R^{t}=0.534 min. ¹H NMR (400 MHZ, METHANOL-d4) δ=8.53 (d, J=2.0 Hz, 1H), 8.49-8.42 (m, 1H), 8.43-8.38 (m, 2H), 7.55-7.40 (m, 5H), 7.37-7.29 (m, 3H), 4.97 (m, 1H), 4.43-4.33 (m, 1H), 4.13-4.02 (m, 1H), 3.97-3.88 (m, 1H), 3.85-3.77 (m, 2H), 3.75-3.67 (m, 2H), 3.66-3.58 (m, 2H), 3.57-3.40 (m, 8H), 3.36 (s, 1H), 3.27-3.22 (m, 1H), 3.17-3.10 (m, 3H), 3.06 (s, 2H), 2.92-2.81 (m, 2H), 2.75 (m, 2H), 2.43 (s, 2H), 2.16-2.02 (m, 2H), 2.01-1.87 (m, 4H), 1.76-1.64 (m, 4H), 1.54 (s, 9H), 1.30 (m, 3H), 1.16-1.06 (m, 3H).

### Step 5: Synthesis of I-194

To a solution of I-194-7 (27 mg, 26.79 μmol, 1 eq) in DCM (0.3 mL) was added TFA (0.6 mL). The mixture was stirred at 25° C. for 1 hr. The reaction mixture was concentrated in vacuo. The residue was purified by reversed phase HPLC (TFA condition), the eluent was concentrated under reduced pressure to remove ACN and lyophilized to give I-194 (5.57 mg, 5.45 μmol, 20.35% yield, 100% purity, TFA) as a white solid. LCMS (Method D): R^{t}=0.316 min, [M+H]⁺=907.3. SFC: R^{t}=1.762 min. ¹H NMR (400 MHz, METHANOL-d4) δ=8.58 (s, 1H), 8.15 (s, 1H), 7.60 (s, 1H), 7.50-7.40 (m, 5H), 7.38-7.25 (m, 3H), 5.31 (m, 1H), 4.60 (m, 1H), 4.18 (d, J=12.8 Hz, 1H), 4.05-3.87 (m, 6H), 3.83 (s, 4H), 3.74-3.58 (m, 8H), 3.53-3.37 (m, 5H), 3.26 (s, 3H), 2.74 (m, 2H), 2.32-2.26 (m, 1H), 2.18 (m, 2H), 2.06 (s, 2H), 2.02-1.88 (m, 3H), 1.70 (d, J=2.8 Hz, 2H), 1.29 (m, 3H), 1.20-1.15 (m, 3H). ¹⁹F NMR (376 MHz, METHANOL-d4) δ=−77.09.

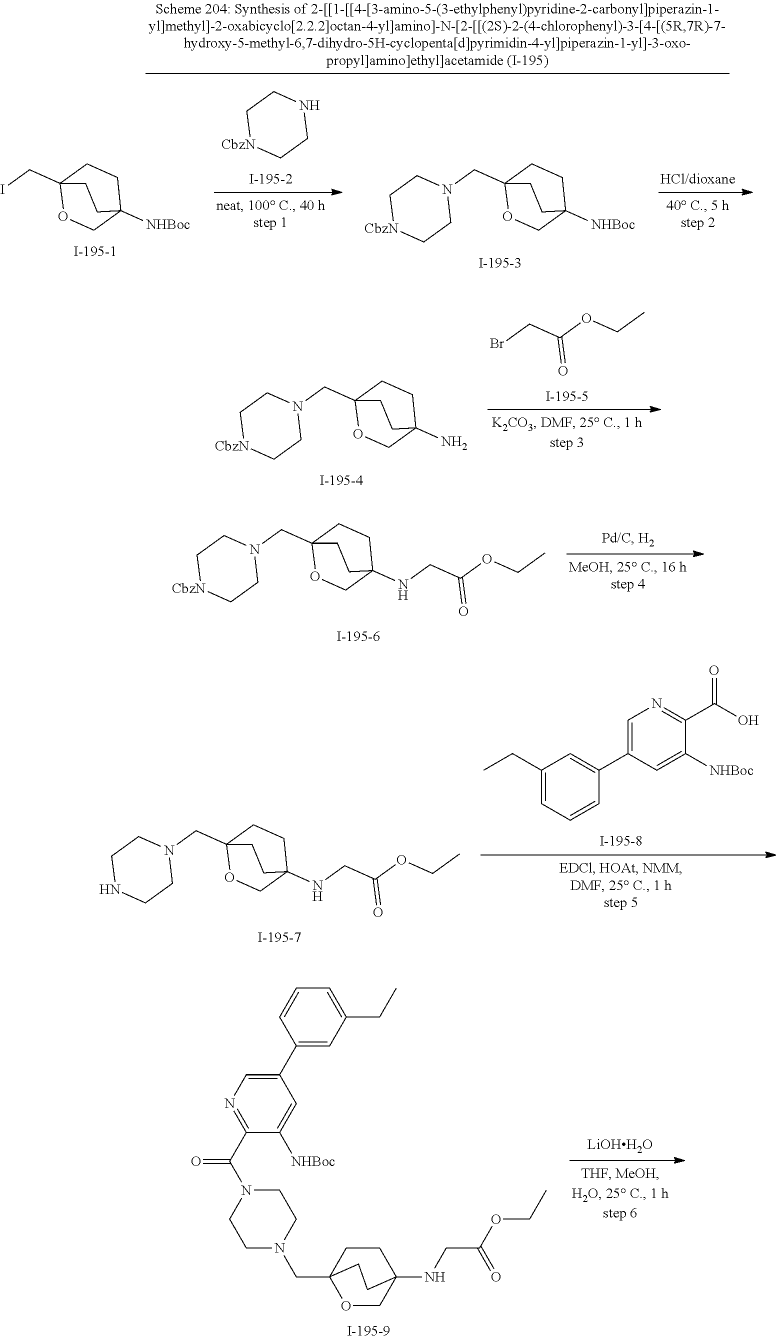

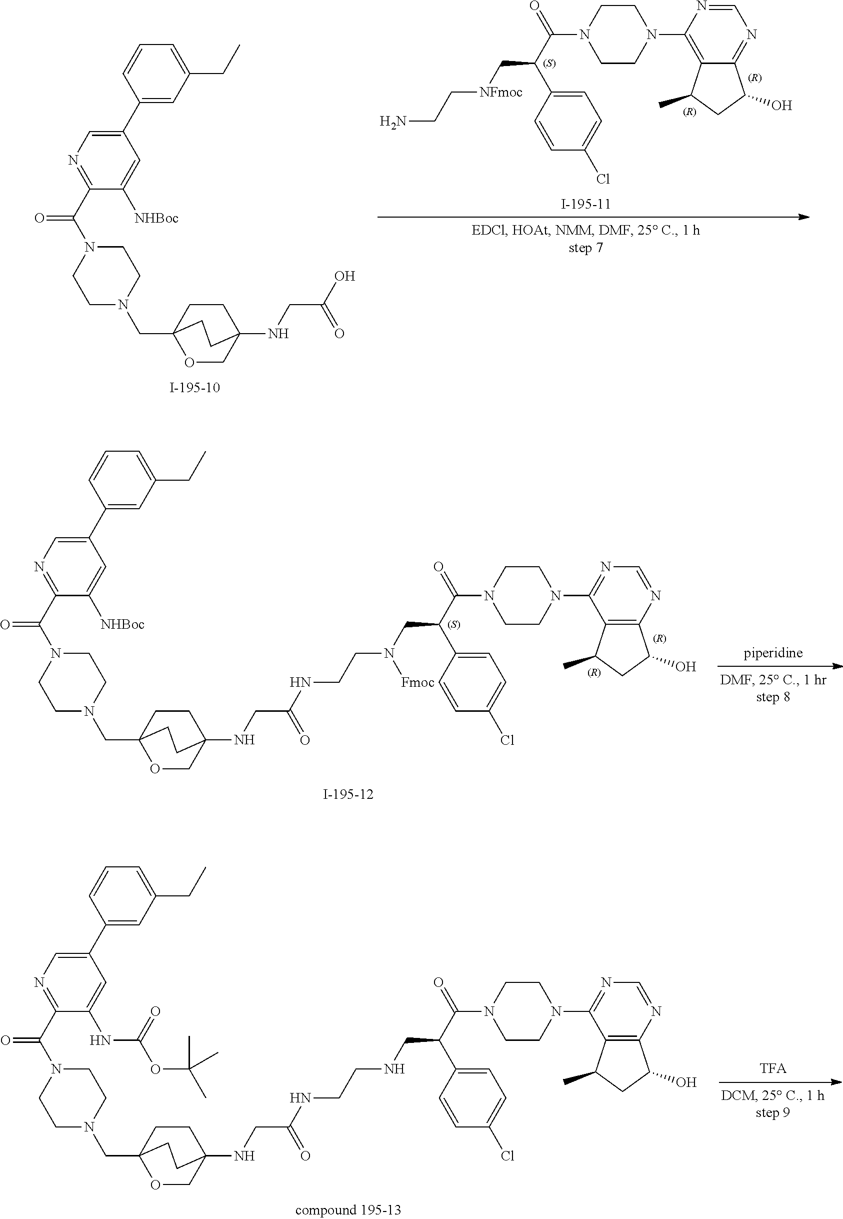

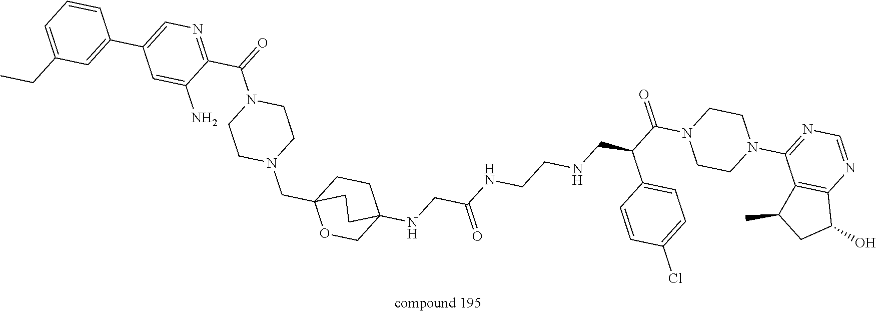 [SMILES: CCc1cccc(-c2cnc(C(=O)N3CCN(CC45CCC(NCC(=O)NCCNC[C@@H](C(=O)N6CCN(c7ncnc8c7[C@H](C)C[C@H]8O)CC6)c6ccc(Cl)cc6)(CC4)CO5)CC3)c(N)c2)c1]

### Step 1: Synthesis of I-195-3

A mixture of I-195-1 (5 g, 13.6 mmol, 1 eq) and I-195-2 (15.00 g, 68.1 mmol, 13.15 mL, 5 eq) was stirred at 100° C. for 40 hours. The reaction mixture was concentrated under reduced pressure to give a residue. The residue was purified by flash silica gel chromatography (ISCO®; 80 g SepaFlash® Silica Flash Column, Eluent of 0˜20% Ethyl acetate/Petroleum ether gradient @ 60 mL/min) and concentrated under reduced pressure to give I-195-3 (2.8 g, 6.03 mmol, 44.30% yield, 99% purity) as a white solid. LCMS: R^{t}=0.308 min, (M+H)=460.3. ¹H NMR (400 MHZ, METHANOL-d4) δ=7.46-7.23 (m, 4H), 5.10 (s, 2H), 3.86 (s, 2H), 3.50-3.43 (m, 4H), 2.52-2.45 (m, 4H), 2.25 (s, 2H), 2.05-1.93 (m, 4H), 1.88-1.79 (m, 2H), 1.70-1.61 (m, 2H), 1.41 (s, 9H).

### Step 2: Synthesis of I-195-4

A solution of I-195-3 (2 g, 4.35 mmol, 1 eq) was added HCl/dioxane (2 M, 20 mL, 9.19 eq). The mixture was stirred at 40° C. for 5 hr. The reaction mixture was concentrated under reduced pressure to give I-195-4 (1.9 g, crude, HCl) as a white solid. LCMS: R^{t}=0.243 min, (M+H)=360.6.

### Step 3: Synthesis of I-195-6

To a mixture of I-195-4 (1.9 g, 5.29 mmol, 1 eq) and I-195-5 (1.06 g, 6.34 mmol, 701.96 UL, 1.2 eq) in DMF (20 mL) was added K₂CO₃ (2.19 g, 15.86 mmol, 3 eq). The mixture was stirred at 25° C. for 1 hr. The reaction mixture was filtered, and concentrated under reduced pressure to give a residue. The residue was purified by flash silica gel chromatography (ISCO®; 80 g SepaFlash® Silica Flash Column, Eluent of 0˜60% Ethyl acetate/Petroleum ether gradient (@ 60 mL/min) and concentrated under reduced pressure to give I-195-6 (2 g, 4.49 mmol, 84.92% yield, N/A purity) as a light-yellow oil. LCMS (Method D): R^{t}=0.160 min, (M+H)=446.2.

### Step 4: Synthesis of I-195-7

A mixture of I-195-6 (2 g, 4.49 mmol, 1 eq), Pd/C (1.00 g, 939.67 μmol, 10% purity; 2.09e-1 eq) in MeOH (20 mL) was degassed and purged with N₂ for 3 times, and then the mixture was stirred at 25° C. for 16 hr under H₂ (15 Psi). The reaction mixture was diluted with MeOH (20 mL*3), filtered, and concentrated under reduced pressure to give a residue. The residue was added ACN and H₂O followed by lyophilization to get I-195-7 (1.2 g, 3.74 mmol, 83.41% yield, 97.168% purity) as a gray solid. LCMS: R^{t}=0.071 min, (M+H)=312.2. ¹H NMR (400 MHZ, METHANOL-d4) δ=4.25-4.12 (m, 2H), 3.65 (s, 2H), 3.39-3.34 (m, 2H), 2.83 (t, J=4.8 Hz, 3H), 2.73-2.65 (m, 1H), 2.60-2.50 (m, 4H), 2.29-2.23 (m, 2H), 2.00-1.92 (m, 2H), 1.81-1.73 (m, 2H), 1.71-1.64 (m, 4H), 1.33-1.25 (m, 3H).

### Step 5: Synthesis of I-195-9

To a solution of I-195-8 (200 mg, 584.13 μmol, 1 eq) and I-195-7 (218.29 mg, 700.96 μmol, 1.2 eq) in DMF (2 mL) was added EDCI (335.94 mg, 1.75 mmol, 3 eq), HOAT (79.51 mg, 584.13 μmol, 81.71 μL, 1 eq) and NMM (295.42 mg, 2.92 mmol, 321.10 μL, 5 eq). The mixture was stirred at 40° C. for 2 hr. The reaction mixture was concentrated under reduced pressure to give a residue. The residue was purified by flash silica gel chromatography (ISCO®; 12 g SepaFlash® Silica Flash Column, Eluent of 0˜100% Ethyl acetate/Petroleum ether gradient @ 40 mL/min) and concentrated under reduced pressure to give I-195-9 (290 mg, 392.27 μmol, 67.15% yield, 86% purity) as a yellow oil. LCMS: R^{t}=0.416 min, (M+H)=636.7. LCMS: R^{t}=0.368 min, (M+H)=636.5.

### Step 6: Synthesis of I-195-10

To a solution of I-195-9 (240 mg, 377.48 μmol, 1 eq) in THF (1 mL), MeOH (1 mL) and H₂O (0.4 mL) was added LiOH·H₂O (79.20 mg, 1.89 mmol, 5 eq). The mixture was stirred at 25° C. for 1 hr. The reaction mixture was concentrated under reduced pressure to give a residue. The crude product was purified by reverse-phase HPLC (Neutral condition) and lyophilized to give I-195-10 (100 mg, 161.25 μmol, 42.72% yield, 98% purity) as a white solid. LCMS (Method D): R^{t}=0.351 min, (M+H)=608.2. ¹H NMR (400 MHZ, METHANOL-d4) δ=8.52 (d, J=2.0 Hz, 1H), 8.42 (d, J=1.6 Hz, 1H), 7.53-7.47 (m, 2H), 7.47-7.34 (m, 2H), 7.32-7.24 (m, 1H), 3.76 (s, 1H), 3.68 (s, 1H), 3.49 (s, 2H), 3.13 (s, 2H), 2.77-2.71 (m, 2H), 2.70-2.64 (m, 2H), 2.59 (d, J=4.4 Hz, 2H), 2.31 (s, 2H), 2.06-1.92 (m, 2H), 1.85-1.61 (m, 6H), 1.55-1.51 (m, 11H), 1.31-1.26 (m, 3H).

### Step 7: Synthesis of I-195-12

To a solution of I-195-10 (60 mg, 98.73 μmol, 1 eq) in DMF (0.6 mL) was added NMM (49.93 mg, 493.63 μmol, 54.27 μL, 5 eq). The mixture was stirred at 25° C. for 5 min. Then I-195-11 (70.85 mg, 98.73 μmol, 1 eq, HCl), EDCI (56.78 mg, 296.18 μmol, 3 eq) and HOAT (13.44 mg, 98.73 μmol, 13.81 μL, 1 eq) were added in and the mixture was stirred at 25° C. for 1 hr. The reaction mixture was concentrated under reduced pressure to give a residue. The crude product was purified by reverse-phase HPLC (Neutral condition) and lyophilized to give I-195-12 (74 mg, 55.90 μmol, 56.62% yield, 96% purity) as a light yellow solid. LCMS (Method D): R^{t}=0.392 min, (M+H)=1270.5. SFC: R^{t}=2.124 min.

### Step 8: Synthesis of I-195-13

To a solution of I-195-12 (20 mg, 15.74 μmol, 1 eq) in DMF (0.1 mL) was added piperidine (2.68 mg, 31.47 μmol, 3.11 μL, 2 eq). The mixture was stirred at 25° C. for 1 hr. The reaction mixture was concentrated under reduced pressure to give a residue. The crude product was purified by reverse-phase HPLC (Neutral condition) and lyophilized to give I-195-13 (9.2 mg, 8.77 μmol, 55.75% yield, 100% purity) as a white solid LCMS (Method D): R^{t}=0.328 min, (M+H)=1048.4. SFC: R^{t}=1.050 min. ¹H NMR (400 MHZ, METHANOL-d4) δ=8.51 (d, J=2.0 Hz, 1H), 8.43-8.37 (m, 2H), 7.53-7.45 (m, 2H), 7.44-7.36 (m, 3H), 7.34-7.28 (m, 3H), 4.96 (t, J=6.8 Hz, 1H), 4.21-4.14 (m, 1H), 3.92-3.84 (m, 1H), 3.78-3.66 (m, 4H), 3.65-3.43 (m, 10H), 3.34 (s, 1H), 3.29 (s, 1H), 3.27-3.23 (m, 1H), 3.17 (s, 2H), 2.77-2.71 (m, 5H), 2.67 (s, 2H), 2.59 (s, 2H), 2.31 (s, 2H), 2.17-2.08 (m, 1H), 2.08-2.02 (m, 1H), 2.02-1.94 (m, 2H), 1.77-1.68 (m, 3H), 1.67-1.61 (m, 3H), 1.54 (s, 9H), 1.29 (t, J=7.6 Hz, 3H), 1.10 (d, J=6.8 Hz, 3H).

### Step 9: Synthesis of I-195

To a solution of I-195-13 (28 mg, 26.70 μmol, 1 eq) in DCM (0.5 mL) was added TFA (460.50 mg, 4.04 mmol, 0.3 mL, 151.26 eq). The mixture was stirred at 25° C. for 1 hr. The reaction mixture was concentrated under reduced pressure to give a residue. The crude product was purified by reverse-phase

HPLC (0.1% TFA condition) and lyophilized to give I-195 (22.15 mg, 20.84 μmol, 78.07% yield, 100% purity, TFA) as a white solid. LCMS (Method D): R^{t}=0.286 min, (M+H)=948.5. SFC: R^{t}=0.868 min. ¹H NMR (400 MHZ, METHANOL-d4) δ=8.58 (s, 1H), 8.15 (s, 1H), 7.53 (d, J=1.6 Hz, 1H), 7.49-7.39 (m, 5H), 7.38-7.35 (m, 2H), 7.29 (d, J=7.2 Hz, 1H), 5.32 (t, J=8.0 Hz, 1H), 4.60-4.54 (m, 1H), 4.27-4.10 (m, 2H), 4.10-3.98 (m, 4H), 3.97-3.87 (m, 3H), 3.86-3.81 (m, 4H), 3.70-3.58 (m, 7H), 3.57-3.48 (m, 3H), 3.45-3.40 (m, 1H), 3.33 (s, 2H), 3.29 (s, 1H), 3.25 (t, J=5.6 Hz, 2H), 2.76-2.69 (m, 2H), 2.33-2.26 (m, 1H), 2.25-2.16 (m, 3H), 2.15-2.06 (m, 4H), 1.98-1.90 (m, 2H), 1.28 (t, J=7.6 Hz, 3H), 1.17 (d, J=7.2 Hz, 3H). F¹⁹F NMR (376 MHz, METHANOL-d4) δ=−76.932.

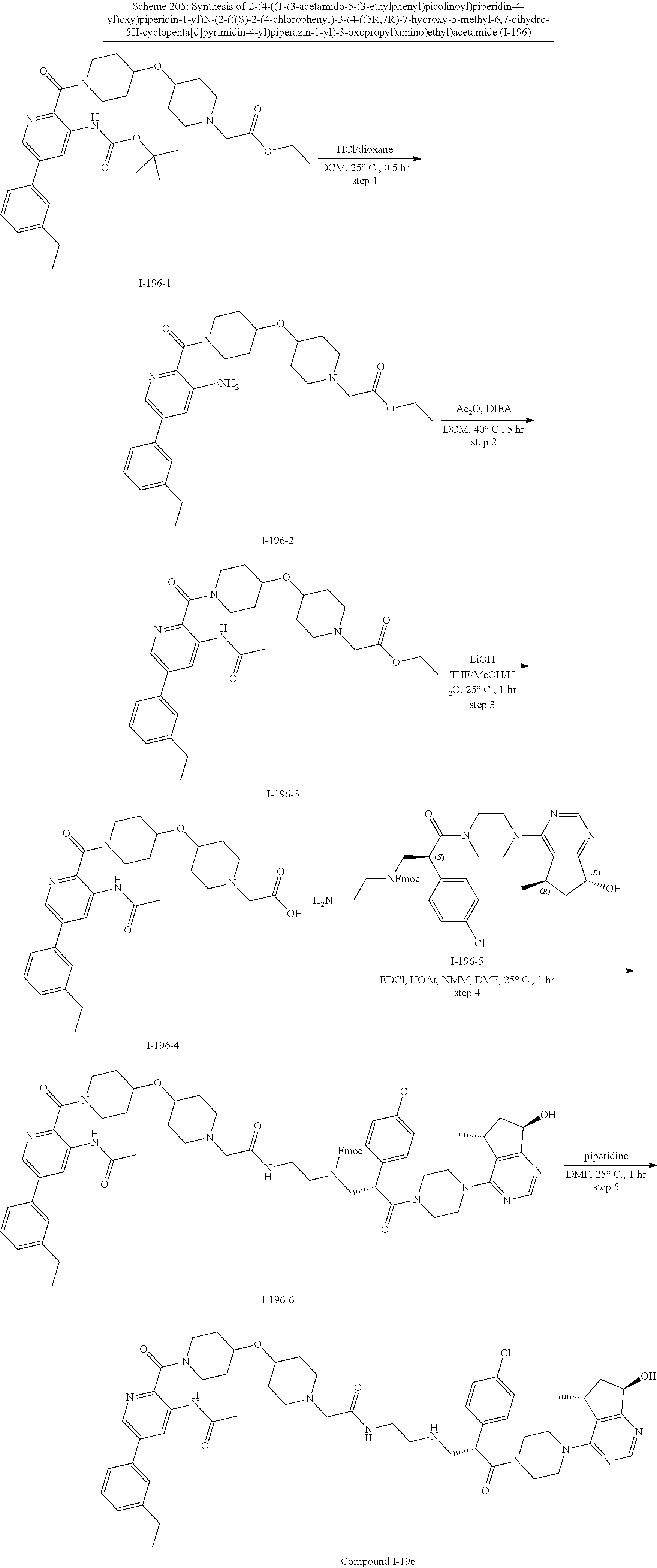

### Step 1: Synthesis of I-196-2

To a solution of I-196-1 (150 mg, 252.21 μmol, 1 eq) in DCM (1.5 mL) was added HCl/dioxane (2 M, 1.5 mL). The mixture was stirred at 25° C. for 1 hr. The reaction mixture was concentrated in vacuo. The crude product was used into the next step without further purification. I-196-2 (120 mg, 225.95 μmol, 89.59% yield, HCl) was obtained as white solid. LCMS (Method D): R^{t}=0.337 min, [M+H]⁺=495.3 ¹H NMR (400 MHZ, METHANOL-d4) δ=8.33 (s, 1H), 8.05-7.99 (m, 1H), 7.61-7.52 (m, 2H), 7.48 (m, 1H), 7.42-7.36 (m, 1H), 4.32 (m, 2H), 4.21-4.16 (m, 2H), 3.97 (s, 1H), 3.84 (m, 2H), 3.69 (d, J=12.8 Hz, 1H), 3.50 (d, J=12.0 Hz, 2H), 3.44-3.38 (m, 1H), 3.29-3.11 (m, 1H), 2.76 (m, 2H), 2.31-2.15 (m, 1H), 2.08 (, 2H), 2.01-1.94 (m, 2H), 1.88-1.78 (m, 1H), 1.72 (s, 2H), 1.33-1.28 (m, 6H), 0.95-0.80 (m, 2H).

### Step 2: Synthesis of I-196-3

To a solution of I-196-2 (100 mg, 188.29 μmol, 1 eq, HCl) in DCM (1 mL) was added DIEA (73.01 mg, 564.88 μmol, 98.39 μL, 3 eq) and Ac2O (28.83 mg, 282.44 μmol, 26.53 μL, 1.5 eq). The mixture was stirred at 40° C. for 1 hr. The mixture was washed with water (3 mL) and extract with DCM (2 mL*3). The combined organic layers dried over sodium sulfate, filtered, and concentrated under reduced pressure to give a residue. The crude product was used into the next step without further purification. I-196-3 (200 mg, crude) was obtained as yellow oil. LCMS (Method D): R^{t}=0.353 min, [M+H]⁺=537.3. ¹H NMR (400 MHz, METHANOL-d4) δ=8.62 (d, J=2.0 Hz, 1H), 8.26 (d, J=2.0 Hz, 1H), 7.54-7.46 (m, 2H), 7.42 (m, 1H), 7.31 (d, J=7.6 Hz, 1H), 4.20 (m, 2H), 4.06-3.99 (m, 1H), 3.84-3.77 (m, 1H), 3.74-3.70 (m, 2H), 3.64 (m, 7.2 Hz, 2H), 3.57-3.51 (m, 1H), 3.44 (s, 2H), 3.23-3.19 (m, 2H), 3.02-2.96 (m, 2H), 2.74 (m, 2H), 2.64 (d, J=2.4 Hz, 2H), 1.79-1.50 (m, 6H), 1.30-1.26 (m, 6H), 1.25-1.20 (m, 2H).

### Step 3: Synthesis of I-196-4

To a solution of I-196-3 (200 mg, 372.67 μmol, 1 eq) in H₂O (0.6 mL), MeOH (0.6 mL), THF (0.6 mL) was added LiOH·H₂O (46.92 mg, 1.12 mmol, 3 eq). The mixture was stirred at 25° C. for 1 hr. The pH was acidified to 3˜4 with 1N HCl, and the mixture was washed with water (3 mL) and extract with EA (3 mL*3). The combined organic layers dried over sodium sulfate, filtered, and concentrated under reduced pressure to give a residue. The residue was purified by reversed phase HPLC (FA condition), the eluent was concentrated under reduced pressure to remove ACN and lyophilized to give I-196-4 (136 mg, 248.68 μmol, 66.73% yield, 93% purity) was obtained as yellow oil. LCMS (Method D): R^{t}=0.338 min, [M+H]⁺=509.2. ¹H NMR (400 MHZ, CHLOROFORM-d) δ=9.69 (s, 1H), 8.94 (s, 1H), 8.53 (s, 1H), 7.46 (s, 2H), 7.43-7.38 (m, 1H), 4.06-4.02 (m, 2H), 3.95-3.87 (m, 3H), 3.73 (d, J=8.4 Hz, 1H), 3.70-3.62 (m, 2H), 3.58-3.47 (m, 2H), 3.47-3.31 (m, 2H), 2.76-2.70 (m, 2H), 2.53-2.45 (m, 1H), 2.24 (s, 3H), 2.04-1.91 (m, 4H), 1.74 (d, J=10.0 Hz, 2H), 0.88 (d, J=7.6 Hz, 5H).

### Step 4: Synthesis of I-196-6

To a solution of I-196-4 (42.52 mg, 83.60 μmol, 1.2 eq) in DMF (0.5 mL) was added HOAt (9.48 mg, 69.67 μmol, 9.75 μL, 1 eq), EDCI (40.07 mg, 209.01 μmol, 3 eq), NMM (35.23 mg, 348.34 μmol, 38.30 μL, 5 eq) and I-196-5 (50 mg, 69.67 μmol, 1 eq, HCl). The mixture was stirred at 25° C. for 0.5 hr. The mixture was washed with water (1 mL) and extract with EA (2 mL*3). The combined organic layers dried over sodium sulfate, filtered, and concentrated under reduced pressure to give a residue. The residue was purified by reversed phase HPLC (FA condition), the eluent was concentrated under reduced pressure to remove ACN and lyophilized to give I-196-6 (65 mg, 55.05 μmol, 79.01% yield, 99.24% purity) was obtained as white solid. LCMS (Method D): R^{t}=0.395 min, [M/2+H]⁺=586.8. SFC: R^{t}=99.035 min, 1.600 min.

### Step 5: Synthesis of I-196

To a solution of I-196-6 (55 mg, 46.94 μmol, 1 eq) in THF (0.6 mL) was added PIPERIDINE (0.075 mL). The mixture was stirred at 25° C. for 1 hr. The mixture was washed with water (1 mL) and extract with EA (2 mL*3). The combined organic layers dried over sodium sulfate, filtered, and concentrated under reduced pressure to give a residue. The residue was purified by prep-HPLC (column: CD07-Daisogel SP-100-8-ODS-PK 150*25*10 um; mobile phase: [water (NH4HCO3)-ACN]; gradient: 34%-64% B over 10 min), the eluent was concentrated under reduced pressure to remove ACN and lyophilized to give I-196 (13.74 mg, 13.75 μmol, 29.30% yield, 99.661% purity, FA) was obtained as a white solid. LCMS (Method D): R^{t}=0.641 min, [M+H]⁺=949.4. SFC: R^{t}=1.512 min. ¹H NMR (400 MHZ, METHANOL-d4) δ=8.62 (s, 1H), 8.41 (s, 1H), 8.24 (d, J=2.0 Hz, 1H), 7.53-7.43 (m, 4H), 7.41-7.25 (m, 4H), 4.97 (m, 1H), 4.47 (d, J=3.2 Hz, 1H), 4.08-3.97 (m, 1H), 3.95-3.87 (m, 1H), 3.85-3.76 (m, 2H), 3.75-3.70 (m, 1H), 3.69-3.60 (m, 3H), 3.59-3.45 (m, 8H), 3.43 (d, J=2.4 Hz, 1H), 3.36-3.32 (m, 1H), 3.24 (d, J=8.4 Hz, 3H), 3.15 (d, J=5.2 Hz, 3H), 2.97 (s, 2H), 2.74 (m, 2H), 2.67-2.52 (m, 2H), 2.17 (s, 2H), 2.14-2.09 (m, 1H), 2.08-2.02 (m, 1H), 2.00-1.88 (m, 4H), 1.70 (m, 4H), 1.29 (m, 3H), 1.10 (d, J=6.8 Hz, 3H).

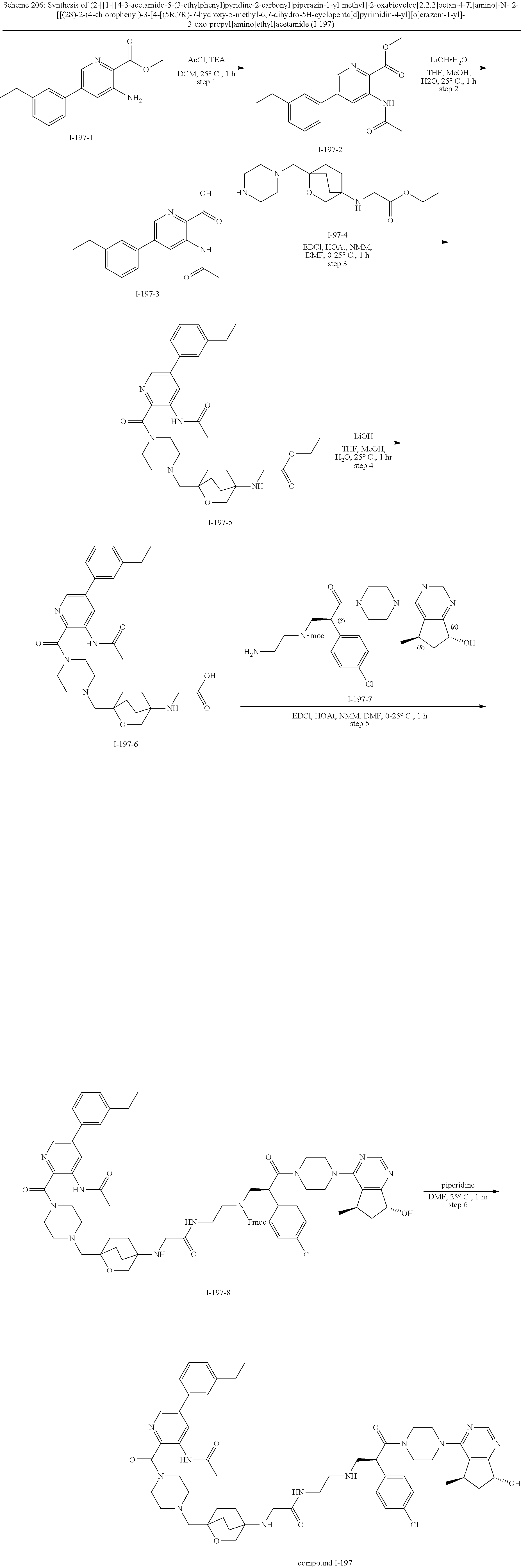

### Step 1: Synthesis of I-197-2

To a solution of I-197-1 (1 g, 3.90 mmol, 1 eq) and acetyl chloride (1.23 g, 15.61 mmol, 1.11 mL, 4 eq) in DCM (10 mL) was added TEA (1.18 g, 11.71 mmol, 1.63 mL, 3 eq). The mixture was stirred at 25° C. for 2 hr. The residue was purified by flash silica gel chromatography (ISCO®; 40 g SepaFlash® Silica Flash Column, Eluent of 0˜35% Ethyl acetate/Petroleum ether gradient @ 60 mL/min). I-197-2 (1.1 g, 3.36 mmol, 86.00% yield, 91% purity) was obtained as a yellow oil. LCMS (Method D): R^{t}=0.489 min, (M+H)=299.4.

### Step 2: Synthesis of I-197-3

To a solution of I-197-2 (550 mg, 1.84 mmol, 1 eq) in THF (2.2 mL), MeOH (2.2 mL) and H₂O (1.1 mL) was added LiOH·H₂O (386.81 mg, 9.22 mmol, 5 eq). The mixture was stirred at 25° C. for 1 hr LC-MS( ). The reaction mixture was concentrated under reduced pressure and adjusted pH=6 with HCl (1 M). Then the mixture was extracted with DCM (5 mL*3). The combined organic layers were dried over Na₂SO₄, filtered, and concentrated under reduced pressure to give I-197-3 (370 mg, 1.30 mmol, 70.59% yield) as a yellow solid. LCMS: R^{t}=0.322 min, (M+H)=285.0.

### Step 3: Synthesis of I-197-5

To a solution of I-197-3 (250 mg, 879.32 μmol, 1 eq) and I-197-4 (328.61 mg, 1.06 mmol, 1.2 eq) in DMF (2.5 mL) was added EDCI (505.70 mg, 2.64 mmol, 3 eq), NMM (444.71 mg, 4.40 mmol, 483.38 μL, 5 eq) and HOAt (119.69 mg, 879.32 μmol, 123.01 μL, 1 eq). The mixture was stirred at 40° C. for 16 hr. The reaction mixture was concentrated under reduced pressure to give a residue. The residue was purified by flash silica gel chromatography (ISCO®; 20 g SepaFlash® Silica Flash Column, Eluent of 0˜76% Ethyl acetate/MeOH @ 36 mL/min) and concentrated under reduced pressure to give I-197-5 (330 mg, 502.67 μmol, 57.17% yield, 88% purity) as a yellow oil. LCMS (Method D): R^{t}=0.321 min, (M+H)=578.7.

### Step 4: Synthesis of I-197-6

To a solution of I-197-5 (310 mg, 536.60 μmol, 1 eq) in THF (3 mL), MeOH (3 mL) and H₂O (1.5 mL) was added LiOH·H₂O (112.58 mg, 2.68 mmol, 5 eq). The mixture was stirred at 25° C. for 1 hr. LC-MS( ). The reaction mixture was filtered, and concentrated under reduced pressure to give a residue. The crude product was purified by reverse-phase HPLC (neutral condition) followed by lyophilization to give I-197-6 (170 mg, 275.26 μmol, 51.30% yield, 89% purity) as a white solid. LCMS (Method D): R^{t}=0.264 min, (M+H)=550.2.

### Step 5: Synthesis of I-197-8

To a solution of I-197-6 (50 mg, 90.97 μmol, 1 eq) and I-197-7 (61.97 mg, 86.34 μmol, 9.49e-1 eq, HCl) in DMF (0.5 mL) was added EDCI (52.31 mg, 272.90 μmol, 3 eq), NMM (46.00 mg, 454.83 μmol, 50.00 μL, 5 eq) and HOAt (12.38 mg, 90.97 μmol, 12.72 μL, 1 eq). The mixture was stirred at 25° C. for 1 hr. The reaction mixture was filtered, and concentrated under reduced pressure to give a residue. The crude product was purified by reverse-phase HPLC (0.1% FA condition) followed by lyophilization to give I-197-8 (50 mg, 37.93 μmol, 41.69% yield, 92% purity) as a yellow solid. LCMS (Method D): R^{t}=0.365 min, (M+H)=607.2.

### Step 6: Synthesis of I-197

To a solution of I-197-8 (40 mg, 32.98 μmol, 1 eq) in DMF (0.4 mL) was added piperidine (5.62 mg, 65.96 μmol, 6.51 μL, 2 eq). The mixture was stirred at 25° C. for 1 hr. LC-MS( ). The reaction mixture was filtered, and concentrated under reduced pressure to give a residue. The crude product was purified by reverse-phase HPLC (0.1% FA condition) followed by lyophilization to give product. LC-MS( ). The crude product was purified by reverse-phase HPLC (neutral condition) followed by lyophilization to give product. LC-MS( ). The crude product was purified by reverse-phase HPLC (neutral condition) followed by lyophilization to give I-197 (2.54 mg, 2.49 μmol, 7.56% yield, 97.195% purity) as a white solid. LCMS (Method D): R^{t}=0.287 min, (M+H)=990.3. ¹H NMR (400 MHZ, METHANOL-d4) δ=8.61 (d, J=2.0 Hz, 1H), 8.41 (s, 1H), 8.28 (d, J=2.0 Hz, 1H), 7.53-7.46 (m, 2H), 7.45-7.41 (m, 1H), 7.40-7.37 (m, 2H), 7.34-7.29 (m, 3H), 4.96 (t, J=6.8 Hz, 1H), 4.20-4.19 (m, 1H), 3.90-3.89 (m, 1H), 3.79-3.70 (m, 4H), 3.62 (s, 3H), 3.59 (d, J=6.4 Hz, 1H), 3.57-3.51 (m, 2H), 3.46 (s, 3H), 3.35-3.33 (m, 2H), 3.28-3.23 (m, 2H), 3.18 (s, 2H), 2.79-2.71 (m, 5H), 2.67 (t, J=5.2 Hz, 2H), 2.62-2.56 (m, 2H), 2.36-2.29 (m, 2H), 2.17 (s, 3H), 2.15-2.09 (m, 1H), 2.17-2.05 (m, 1H), 2.02-1.94 (m, 2H), 1.74-1.62 (m, 6H), 1.29 (t, J=7.6 Hz, 3H), 1.11 (d, J=6.8 Hz, 3H).

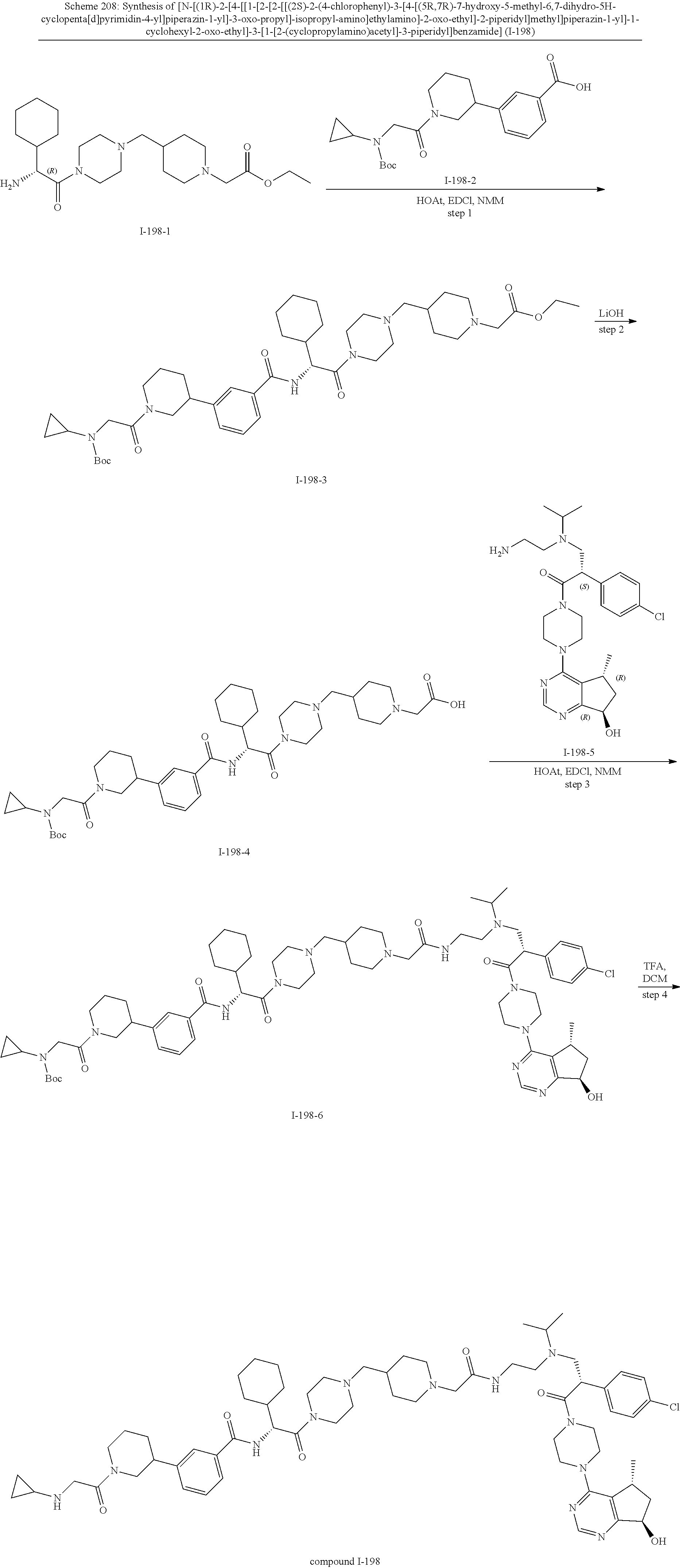

### Step 1: Synthesis of I-198-3

To a solution of I-198-2 (100 mg, 224.70 μmol, 1 eq, HCl) in DMF (1 mL) was added EDCI (129.23 mg, 674.10 μmol, 3 eq), NMM (113.64 mg, 1.12 mmol, 123.52 μL, 5 eq), I-198-1 (90.44 mg, 224.70 μmol, 1 eq) and HOAt (30.58 mg, 224.70 μmol, 31.43 μL, 1 eq). The mixture was stirred at 25° C. for 1 h. The crude product was purified by prep-HPLC (0.1% FA condition) and the eluent was lyophilized to give I-198-3 (120 mg, 148.29 μmol, 65.99% yield, 98% purity) as a white solid. SFC: R^{t}=1.641 min LCMS (Method D): R^{t}=0.358 min, (M+H)=793.5.

### Step 2: Synthesis of I-198-4

The I-198-3 (110 mg, 138.71 μmol, 1 eq) in MeOH (0.4 mL), THF (0.4 mL) and H₂O (0.4 mL) was added LiOH·H₂O (17.46 mg, 416.12 μmol, 3 eq). The mixture was stirred at 25° C. for 0.5 h. Concentrated under reduced pressure to give I-198-4 (110 mg, crude) as a white solid. SFC: R^{t}=2.353 min, 2.765 min LCMS (Method D): R^{t}=0.409 min, (M+H)=765.9.

### Step 3: Synthesis of I-198-6

To a solution of I-198-4 (62.19 mg, 81.29 μmol, 1 eq) in DMF (1 mL) was added EDCI (46.75 mg, 243.87 μmol, 3 eq), HOAt (11.06 mg, 81.29 μmol, 11.37 μL, 1 eq) and NMM (41.11 mg, 406.45 μmol, 44.69 μL, 5 eq) and I-198-5 (50 mg, 81.29 μmol, 1 eq, TFA). The mixture was stirred at 25° C. for 12 h. The crude product was purified by prep-HPLC (0.1% NH₃·H₂O condition) and the eluent was lyophilized to give I-198-6 (80 mg, 62.82 μmol, 77.28% yield, 98% purity) as a white solid. SFC: R^{t}=0.607 min. LCMS: R^{t}=0.803 min, (M+H)=1248.2. LCMS: R^{t}=0.814 min, (M+H)=1248.3.

### Step 4: Synthesis of 1-198

To a solution of I-198-6 (70 mg, 56.09 μmol, 1 eq) in DCM (0.7 mL) was added TFA (214.90 mg, 1.88 mmol, 140.00 μL, 33.60 eq). The mixture was stirred at 25° C. for 0.5 h. Concentrated under reduced pressure to give a residue. The crude product was purified by prep-HPLC (0.1% TFA condition) and the eluent was lyophilized to give I-198 (46.98 mg, 36.57 μmol, 65.20% yield, 98.232% purity, TFA) as a white solid. SFC: R^{t}=3.292 min, 3.706 min LCMS (Method D): R^{t}=0.322 min, (M+H)=1147.8. 1H NMR (400 MHZ, METHANOL-d4) δ=8.58 (s, 1H), 7.86-7.63 (m, 2H), 7.59-7.32 (m, 6H), 5.31 (t, J=8.0 Hz, 1H), 4.85-4.69 (m, 5H), 4.61-4.50 (m, 1H), 4.35-4.12 (m, 4H), 4.06-3.62 (m, 16H), 3.55-3.38 (m, 6H), 3.20 (s, 6H), 3.02-2.59 (m, 4H), 2.32-2.26 (m, 2H), 2.21-2.02 (m, 4H), 2.00-1.84 (m, 4H), 1.84-1.61 (m, 7H), 1.45 (s, 3H), 1.38-1.24 (m, 6H), 1.21-1.06 (m, 5H), 1.00-0.81 (m, 4H).

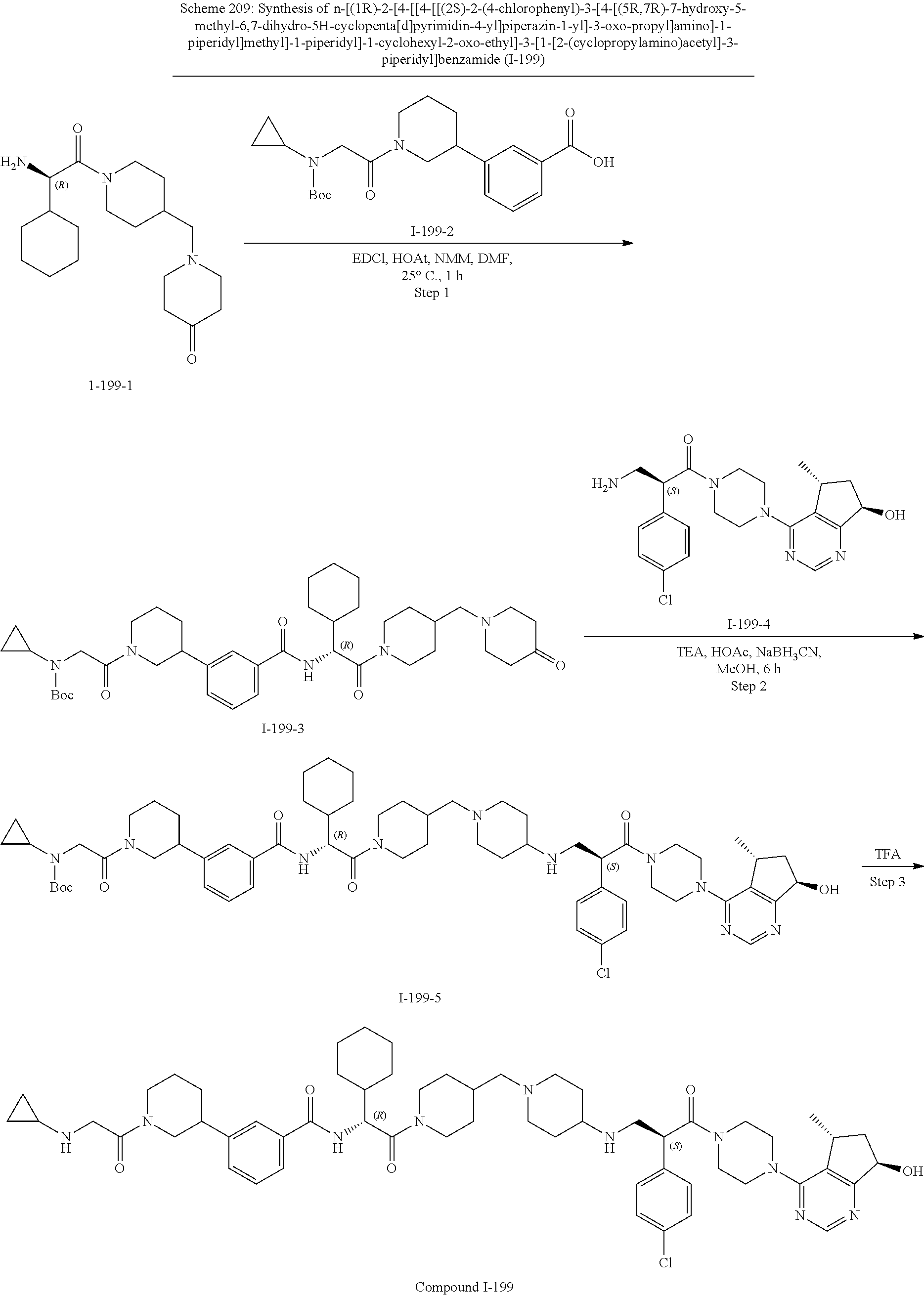

### Step 1: Synthesis of I-199-3

To a solution of I-199-1 (50 mg, 134.43 μmol, 1 eq, HCl) and I-199-2 (59.52 mg, 147.87 μmol, 1.1 eq) in DMF (0.5 mL) was added EDCI (77.31 mg, 403.29 μmol, 3 eq) and HOAt (18.30 mg, 134.43 μmol, 18.80 μL, 1 eq), NMM (67.99 mg, 672.14 μmol, 73.90 μL, 5 eq). The mixture was stirred at 25° C. for 1 hr. The mixture was diluted with water (5 mL) and extracted with DCM (5 mL*3). The combined organic layer was dried over Na₂SO₄, filtered, and concentrated in vacuo to give a residue. The residue was purified by prep-HPLC (column: CD01-Phenomenex luna C18 150*25*10 um; mobile phase: [water (FA)-ACN]; gradient: 22%-52% B over 11 min). Then eluent was concentrated in vacuo and lyophilized. I-199-3 (40 mg, 52.32 μmol, 38.92% yield, 94.176% purity) was obtained as yellow oil. LCMS (Method D): R^{t}=0.416 min, (M+H)=720.6. SFC: R^{t}=0.888 min, 1.305 min.

### Step 2: Synthesis of I-199-5

To a solution of I-199-4 (10.68 mg, 23.61 μmol, 1 eq, HCl) in MeOH (0.5 mL) was added dropwise TEA (9.56 mg, 94.45 μmol, 13.15 μL, 4 eq) at 25° C. for 10 min. Then I-199-3 (17 mg, 23.61 μmol, 1 eq) and HOAc (5.67 mg, 94.45 μmol, 5.41 μL, 4 eq) as added at 25° C. for 6 h. After addition, NaBH₃CN (8.90 mg, 141.68 μmol, 6 eq) was added. The resulting mixture was stirred at 25° C. for 1 hr. The mixture was diluted with water (5 mL) and extracted with DCM (5 mL*3). The combined organic layer was dried over Na₂SO₄, filtered, and concentrated in vacuo to give a residue. The residue was purified by reverse phase (0.1% FA condition), then the residue was concentrated in vacuo and lyophilized. I-199-5 (10 mg, 8.22 μmol, 34.79% yield, 92% purity) was obtained as a white solid. LCMS (Method D): R^{t}=0.396 min, (M+H)=1119.7.

### Step 3: Synthesis of I-199

To a solution of I-199-5 (10 mg, 8.93 μmol, 1 eq) in DCM (0.4 mL) was added TFA (153.50 mg, 1.35 mmol, 0.1 mL, 150.76 eq). The mixture was stirred at 25° C. for 0.5 hr. The reaction mixture was concentrated in vacuo to give a residue. The residue was purified by reverse phase (0.1% TFA condition), then the residue was concentrated in vacuo and lyophilized. I-199 (4.75 mg, 4.19 μmol, 46.92% yield, 100% purity, TFA) was obtained as a white solid. R^{t}=4.274 min, 5.947 min. ¹H NMR (400 MHZ, METHANOL-d4) δ=8.57 (s, 1H), 8.38-8.23 (m, 1H), 7.82-7.67 (m, 2H), 7.57-7.41 (m, 4H), 7.37 (d, J=8.0 Hz, 2H), 5.28 (t, J=7.2 Hz, 1H), 4.92 (d, J=7.6 Hz, 1H), 4.65-4.48 (m, 3H), 4.40-4.32 (m, 1H), 4.25 (d, J=13.4 Hz, 1H), 4.20-4.13 (m, 1H), 4.09-3.99 (m, 1H), 3.89 (d, J=2.8 Hz, 1H), 3.84 (s, 2H), 3.80-3.73 (m, 3H), 3.71-3.58 (m, 3H), 3.53-3.40 (m, 2H), 3.28-3.19 (m, 3H), 3.15-3.00 (m, 4H), 2.94-2.84 (m, 1H), 2.83-2.71 (m, 3H), 2.49-2.34 (m, 2H), 2.31-2.25 (m, 1H), 2.23-2.10 (m, 4H), 2.09-1.98 (m, 2H), 1.89 (s, 5H), 1.77 (s, 2H), 1.73-1.67 (m, 2H), 1.35-1.25 (m, 6H), 1.23-1.16 (m, 4H), 1.15-1.05 (m, 2H), 0.92 (d, J=8.4 Hz, 5H).

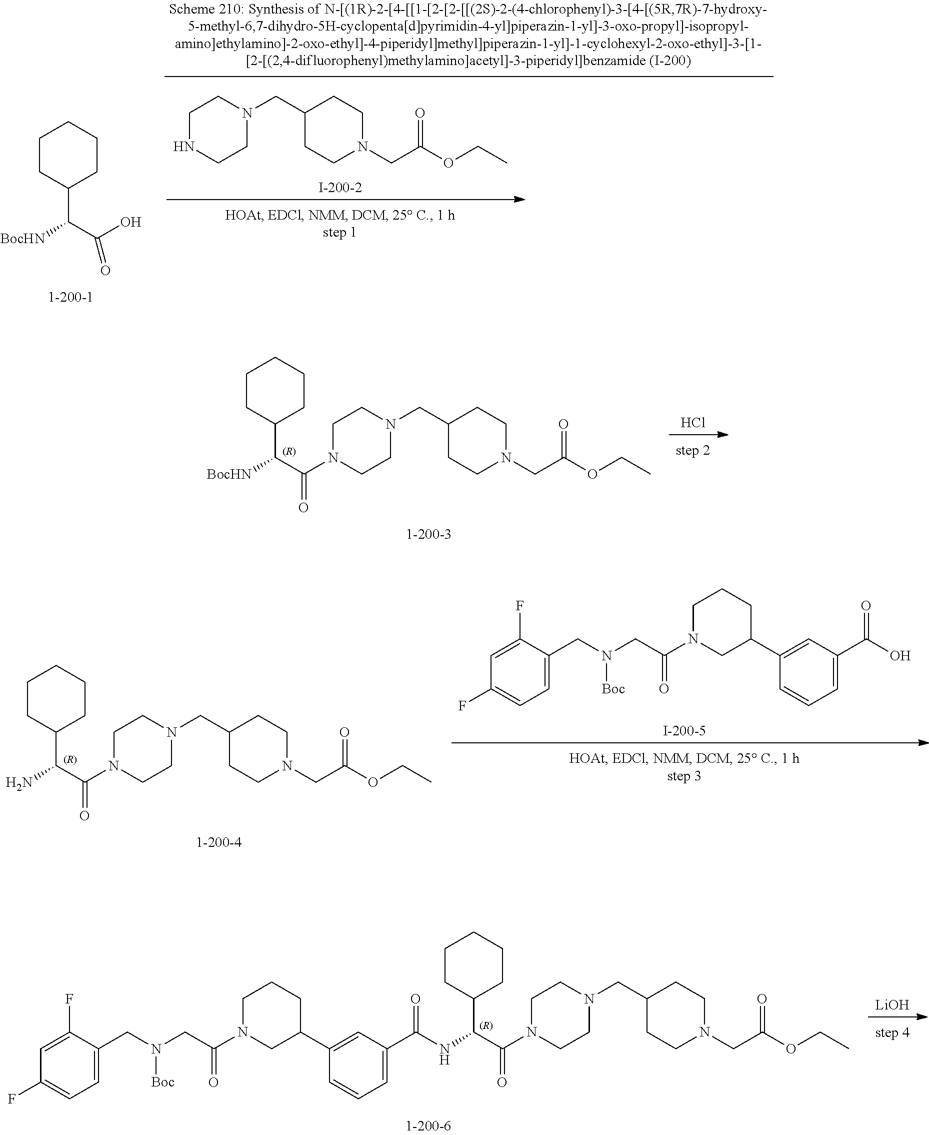

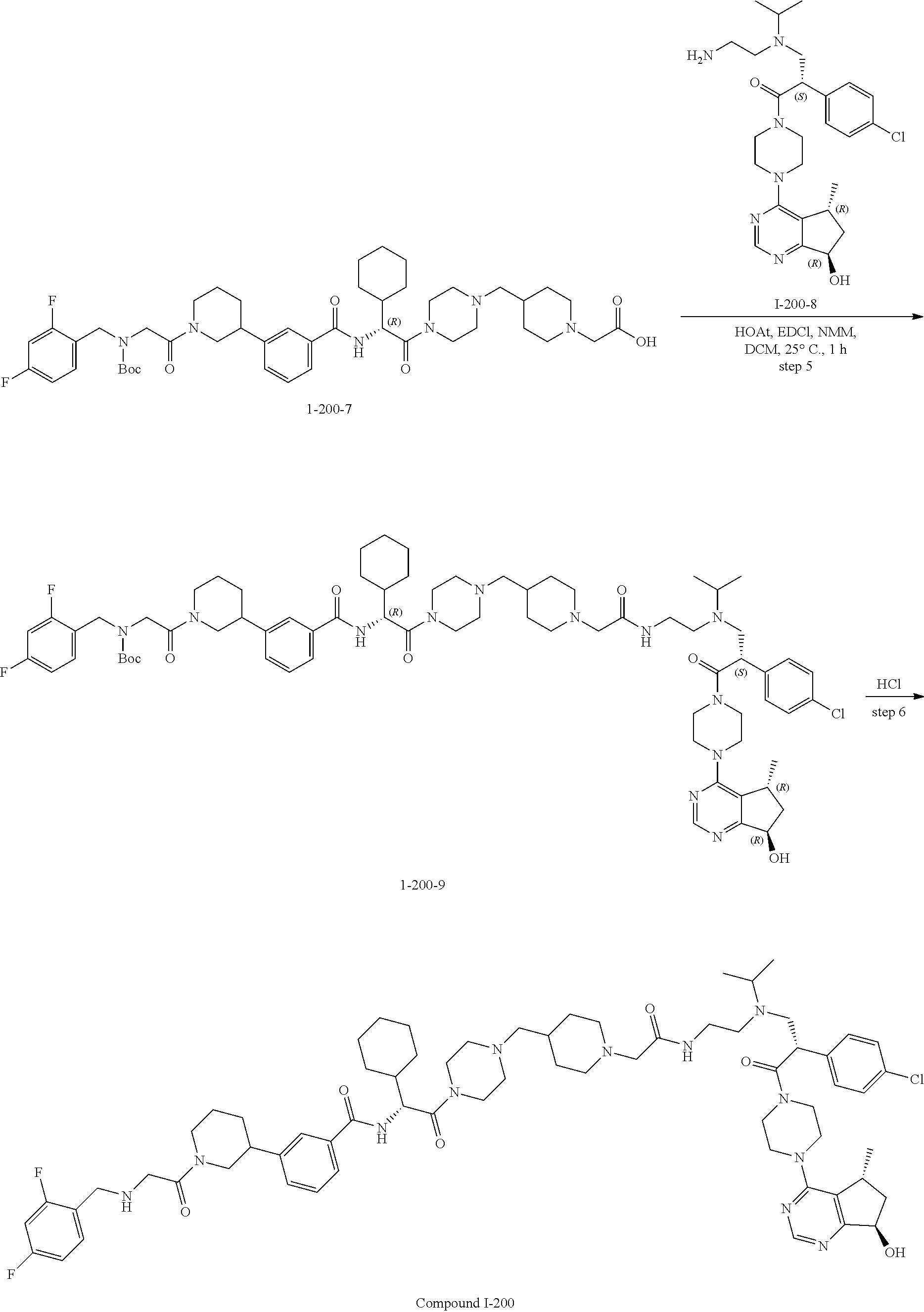

### Step 1: Synthesis of I-200-3

To a solution of I-200-1 (191.05 mg, 742.44 μmol, 1 eq) in DCM (4 mL) was added EDCI (426.98 mg, 2.23 mmol, 3 eq), NMM (375.49 mg, 3.71 mmol, 408.14 μL, 5 eq) and HOAt (101.05 mg, 742.44 μmol, 103.86 μL, 1 eq) at 25° C. for 0.5 hr. And then I-200-2 (200 mg, 742.44 μmol, 1 eq) was added into mixture and stirred at 25° C. for 0.5 hr. The reaction mixture was diluted with water (60 mL) and extracted with ethyl acetate (20 mL×3). The combined organic layers were washed with brine (20 mL×3) and dried over sodium sulfate, filtered, and concentrated under reduced pressure to give a residue. The residue was purified by flash silica gel chromatography (ISCO®; 20 g SepaFlash R: Silica Flash Column, Eluent of 0˜40% Methanol: Ethyl acetate gradient (@, 40 mL/min). The eluent was concentrated under reduced pressure to give I-200-3 (240 mg, 435.75 μmol, 58.69% yield, 92.359% purity) as a yellow oil. LCMS (Method D): R^{t}=0.275 min, (M+H)=509.3.4.

### Step 2: Synthesis of I-200-4

To a solution of I-200-3 (200 mg, 393.17 μmol, 1 eq) in DCM (2 mL) was added HCl/dioxane (2 M, 196.58 μL). The mixture was stirred at 25° C. for 0.5 hr. The mixture was concentrated under reduced pressure to give I-200-4 (150 mg, 367.13 μmol, 93.38% yield) as a yellow oil. LCMS (Method D): R^{t}=0.243 min, (M+H)=409.2.

### Step 3: Synthesis of I-200-6

To a solution of I-200-5 (119.57 mg, 244.75 μmol, 1 eq) in DCM (5 mL) was added EDCI (140.76 mg, 734.26 μmol, 3 eq), NMM (123.78 mg, 1.22 mmol, 134.54 μL, 5 eq) and HOAt (33.31 mg, 244.75 μmol, 34.24 μL, 1 eq) at 25° C. for 0.5 hr. And then I-200-4 (100 mg, 244.75 μmol, 1 eq) was added into mixture and stirred at 25° C. for 0.5 hr. The reaction mixture was diluted with water (30 mL) and extracted with ethyl acetate (10 mL×3). The combined organic layers were washed with brine (10 mL×3) and dried over sodium sulfate, filtered, and concentrated under reduced pressure to give a residue. The crude product was purified by reverse-phase HPLC (0.1% FA condition) and the eluent was concentrated under reduced pressure to remove ACN and then lyophilized to give I-200-6 (200 mg, 226.95 μmol, 92.72% yield, 99.752% purity) as a white solid. LCMS (Method D): R^{t}=0.395 min, (M+H)=879.4. ¹H NMR (400 MHz, CHLOROFORM-d) δ=7.71-7.64 (m, 2H), 7.43-7.29 (m, 3H), 7.03-6.96 (m, 1H), 6.90-6.77 (m, 2H), 5.01 (s, 1H), 4.74-4.45 (m, 3H), 4.23-4.18 (m, 1H), 4.16-3.86 (m, 3H), 3.84-3.69 (m, 5H), 3.67-3.60 (m, 1H), 3.40 (d, J=8.8 Hz, 2H), 3.24-3.02 (m, 4H), 2.82 (s, 1H), 2.78-2.72 (m, 1H), 2.69-2.41 (m, 10H), 2.40-2.27 (m, 3H), 2.15-1.99 (m, 2H), 1.90-1.71 (m, 10H), 1.70-1.60 (m, 3H), 1.55 (s, 1H), 1.36-1.04 (m, 9H). ¹⁹F NMR (376 MHZ, METHANOL-d4) δ=−111.382, −113.379, −113.401. SFC: R^{t}=1.813 min.

### Step 4: Synthesis of I-200-7

To a solution of I-200-6 (200 mg, 227.51 μmol, 1 eq) in THF (0.8 mL), H₂O (0.2 mL) and MeOH (0.8 mL) was added LiOH·H₂O (28.64 mg, 682.53 μmol, 3 eq). The mixture was stirred at 40° C. for 0.5 hr. The mixture was concentrated to give a residue. The reaction mixture was diluted with water (30 mL). Then the mixture was adjusted to pH=6˜7 with saturated citric acid aqueous solution. Then the reaction mixture was extracted with DCM (10 mL×3). The combined organic layers were washed with brine (10 mL×3) and dried over sodium sulfate, filtered, and concentrated under reduced pressure to give I-200-7 (160 mg, 185.82 μmol, 81.68% yield, 98.839% purity) as a white solid. LCMS (Method D): R^{t}=0.399 min, (M+H)=851.3.

### Step 5: Synthesis of I-200-9

To a solution of I-200-7 (30 mg, 35.25 μmol, 1 eq) in DCM (3 mL) was added EDCI (20.27 mg, 105.75 μmol, 3 eq). NMM (17.83 mg, 176.26 μmol, 19.38 μL, 5 eq) and HOAt (4.80 mg, 35.25 μmol, 4.93 μL, 1 eq) at 25° C. for 0.5 hr. And then I-200-8 (17.66 mg, 35.25 μmol, 1 eq) was added into mixture and stirred at 25° C. for 0.5 hr. The reaction mixture was diluted with water (21 mL) and extracted with ethyl acetate (7 mL×3). The combined organic layers were washed with brine (7 mL×3) and dried over sodium sulfate, filtered, and concentrated under reduced pressure to give a residue. The residue was purified by flash silica gel chromatography (ISCO®; 4 g SepaFlash® Silica Flash Column, Eluent of 0˜30% Methanol: DCM (@ 40 mL/min). The eluent was concentrated under reduced pressure to give I-200-9 (20 mg, 8.42 μmol, 23.90% yield, 56.198% purity) as a yellow oil. LCMS (Method D): R^{t}=0.368 min, (M+H)=1333.4.1.

### Step 6: Synthesis of I-200

To a solution of I-200-9 (10 mg, 7.50 μmol, 1 eq) in DCM (0.5 mL) was added TFA (30.70 mg, 269.24 μmol, 20.00 μL, 35.92 eq). The mixture was stirred at 25° C. for 0.5 hr. The mixture was concentrated under reduced pressure to give a residue. The crude product was purified by reverse-phase HPLC (0.1% TFA condition) and the eluent was concentrated under reduced pressure to remove ACN and then lyophilized to give I-200 (3.06 mg, 2.27 μmol, 30.28% yield, 100% purity, TFA) as a white solid., and LCMS (Method D): R=0.291 min, (M+H)=1234.2. ¹H NMR (400 MHZ, METHANOL-d4) δ=8.58 (s, 1H), 7.81-7.72 (m, 2H), 7.66-7.57 (m, 1H), 7.55-7.45 (m, 4H), 7.42-7.36 (m, 2H), 7.20-7.05 (m, 2H), 5.30 (s, 1H), 4.76-4.71 (m, 1H), 4.65-4.49 (m, 2H), 4.32 (d, J=12.8 Hz, 3H), 4.26 (d, J=5.6 Hz, 1H), 4.23-4.17 (m, 2H), 4.16-4.00 (m, 3H), 3.98-3.88 (m, 4H), 3.82-3.61 (m, 10H), 3.52-3.39 (m, 6H), 3.27 (s, 1H), 3.24-3.20 (m, 2H), 3.16 (s, 3H), 2.99-2.72 (m, 3H), 2.35-2.23 (m, 2H), 2.21-2.02 (m, 5H), 1.98-1.87 (m, 4H), 1.79 (d, J=10.2 Hz, 3H), 1.72 (d, J=8.4 Hz, 4H), 1.49-1.41 (m, 3H), 1.38-1.27 (m, 6H), 1.23-1.08 (m, 6H). ¹⁹F NMR (376 MHz, METHANOL-d4) δ=−77.225, −108.410, −112.868. SFC: R^{t}=2.004 min, 2.215 min.

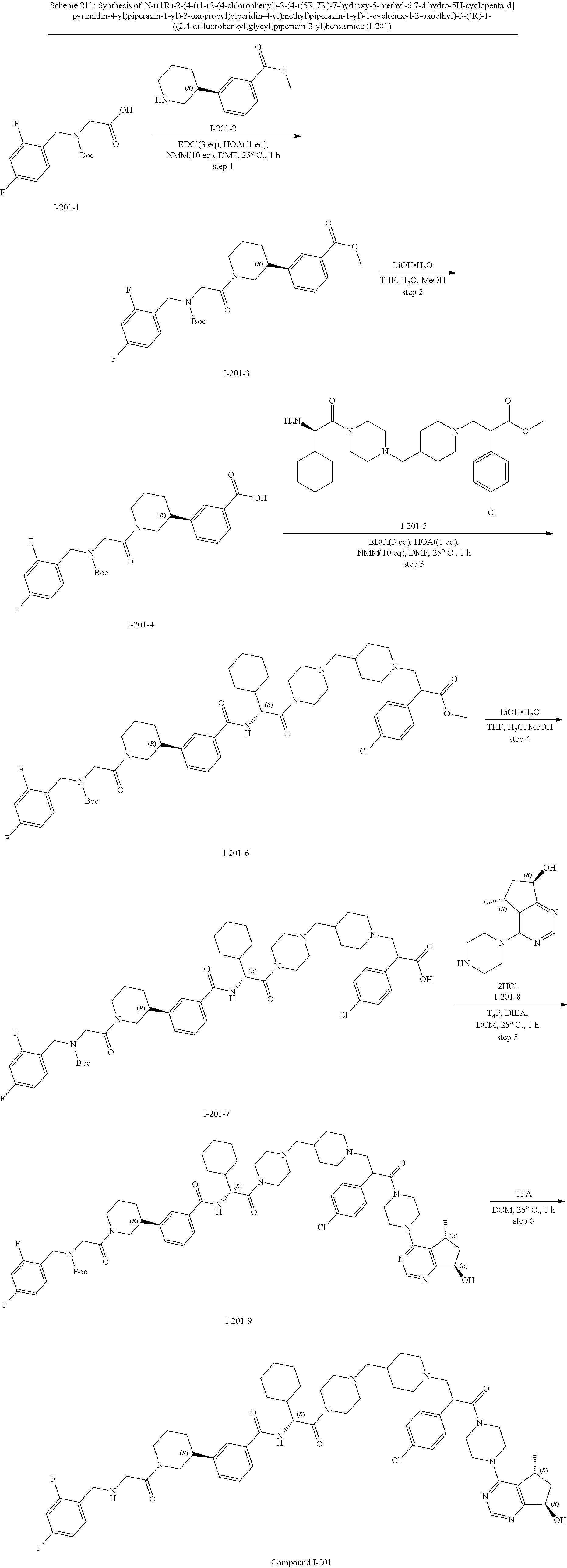

### Step 1: Synthesis of I-201-3

To a solution of I-201-2 (373.49 mg, 1.46 mmol, 1.1 eq, HCl) and I-201-1 (400 mg, 1.33 mmol, 1 eq) in DMF (5 mL) was added EDCI (763.53 mg, 3.98 mmol, 3 eq), HOAt (180.71 mg, 1.33 mmol, 185.72 μL, 1 eq) and NMM (1.34 g, 13.28 mmol, 1.46 mL, 10 eq). The mixture was stirred at 25° C. for 1 hr. The reaction mixture was quenched by addition H₂O (10 mL), and then diluted with EA (10 mL) and extracted with EA 30 mL (10 mL*3). The combined organic layers were washed with brine 30 mL (10 mL*3), dried over anhydrous Na₂SO₄, filtered, and concentrated under reduced pressure to give a residue. The residue was purified by flash silica gel chromatography (ISCO®; 20 g SepaFlash® Silica Flash Column, Eluent of 0˜100% Ethyl acetate/Petroleum ether gradient @ 40 mL/min), concentrated under reduced pressure to give a product. I-201-3 (600 mg, 1.17 mmol, 88.13% yield, 98% purity) was obtained as a white solid. LCMS: R^{t}=0.602 min, [M+H]⁺=503.4.

### Step 2: Synthesis of I-201-4

To a solution of I-201-3 (350 mg, 696.45 μmol, 1 eq) in THF (3.5 mL), H₂O (3.5 mL), MeOH (3.5 mL) was added LiOH·H₂O (87.68 mg, 2.09 mmol, 3 eq). The mixture was stirred at 25° C. for 2 hr. The reaction mixture was adjusted to pH=6 with HCl (1M) and then extracted with EA 15 mL (15 mL*3). The combined organic layers were dried over anhydrous Na₂SO₄, filtered, and concentrated under reduced pressure to give a residue. The crude product was used into the next step without further purification. I-201-4 (330 mg, crude) was obtained as a white solid. LCMS: R^{t}=0.408 min, [M+Na]⁺=511.3.

### Step 3: Synthesis of I-201-6

To a solution of I-201-4 (330 mg, 675.51 μmol, 1 eq), I-201-5 (350.67 mg, 675.51 μmol, 1 eq) in DMF (3 mL) was added EDCI (388.49 mg, 2.03 mmol, 3 eq) and HOAt (91.94 mg, 675.51 μmol, 94.50 μL, 1 eq), NMM (683.25 mg, 6.76 mmol, 742.67 μL, 10 eq). The mixture was stirred at 25° C. for 1 hr. The reaction mixture was quenched by addition water (5 mL) at 25° C., and extracted with EA (5 mL*3). The combined organic layers were washed with sat. NaCl (5 mL*3), dried over Na₂SO₄, filtered, and concentrated under reduced pressure to give a residue. The crude product (520 mg, crude) was used into the next step without further purification. I-201-6 (520 mg, crude) was obtained as a white solid. LCMS: R^{t}=0.911 min, [M+H-Boc]⁺=889.6.

### Step 4: Synthesis of I-201-7

To a solution of I-201-6 (500 mg, 505.24 μmol, 1 eq) in H₂O (5 mL), THF (5 mL), MeOH (5 mL) was added LiOH·H₂O (63.61 mg, 1.52 mmol, 3 eq). The mixture was stirred at 25° C. for 1 hr. LC-MS( ) showed Reactant 1 was consumed completely. The reaction mixture was adjusted to pH to 6 with HCl (1M) and then extracted with EA 15 mL (15 mL*3). The combined organic layers were dried over anhydrous Na₂SO₄, filtered, and concentrated under reduced pressure to give a residue. The residue was purified by reversed phase (26.8*125 mm, 80 g of XB—C18, 20-40 μm, 120 Å) Mobile phase: A for H₂O (0.1% FA v/v) and B for acetonitrile; Gradient: B 30%-50% in 30 min; Flow rate: 60 ml/min; Column temperature: R.T. Wavelength: 220 nm/254 nm), after purification, then concentrated to remove organic solvents and then lyophilization. I-201-7 (320 mg, 308.32 μmol, 61.02% yield, 94% purity) was obtained as a white solid. LCMS: R^{t}=0.627 min, [M+H]⁺=975.7. SFC: R^{t}=0.672 min, 0.921 min.

### Step 5: Synthesis of I-201-9

To a solution of I-201-7 (300 mg, 307.50 μmol, 1 eq), I-201-8 (94.47 mg, 307.50 μmol, 1 eq, 2HCl) in DCM (2.5 mL) was added T₄P (221.56 mg, 615.01 μmol, 2 eq) and DIEA (119.23 mg, 922.51 μmol, 160.68 μL, 3 eq). The mixture was stirred at 25° C. for 1 hr. The reaction mixture was combined with another batch for work-up. The mixture was quenched by addition water (5 mL) at 25° C., and extracted with EA (5 mL*3). The combined organic layers were washed with sat. NaCl (5 mL*3), dried over Na₂SO₄, filtered, and concentrated under reduced pressure to give a residue. The product was purified by prep-HPLC (column: Waters Xbridge Prep OBD C18 150*40 mm*10 um; mobile phase: [water (NH₄HCO₃)-ACN]; gradient: 62%-92% B over 15 min) and the eluent was concentrated to remove MeCN and then lyophilization. I-201-9 (70 mg, 50.51 μmol, 54.75% yield, 86% purity) was obtained as a white solid. LCMS: R^{t}=0.607 min, [M+H]⁺=975.7.

### Step 6: Synthesis of I-201

To a solution of I-201-9 (70 mg, 58.73 μmol, 1 eq) in DCM (0.5 mL) was added TFA (805.88 mg, 7.07 mmol, 525.00 μL, 120.34 eq). The mixture was stirred at 25° C. for 1 hr. The reaction mixture was concentrated under reduced pressure to give a residue. The product was purified by prep-HPLC (column: Phenomenex luna C18 150*25 mm*10 um; mobile phase: [water (TFA)-ACN]; gradient: 3%-33% B over 10 min) and the eluent was concentrated to remove MeCN and then lyophilization. I-201 (60 mg, 49.26 μmol, 83.88% yield, 99% purity, TFA) was obtained as a white solid. LCMS: R^{t}=0.797 min, [M+H]⁺=1091.8. SFC: R^{t}=1.191 min, 2.284 min. ¹H NMR (400 MHZ, METHANOL-d4) δ=8.58 (d, J=2.8 Hz, 1H), 7.81-7.70 (m, 2H), 7.67-7.57 (m, 1H), 7.56-7.41 (m, 4H), 7.41-7.32 (m, 2H), 7.19-7.04 (m, 2H), 5.30 (m, 1H), 4.83 (m, 2H), 4.76-4.67 (m, 1H), 4.63-4.51 (m, 1H), 4.36-4.17 (m, 5H), 4.16-3.92 (m, 5H), 3.91-3.60 (m, 8H), 3.58-3.36 (m, 5H), 3.30-2.97 (m, 7H), 2.93-2.70 (m, 2H), 2.34-2.02 (m, 6H), 2.01-1.84 (m, 4H), 1.83-1.58 (m, 7H), 1.41-1.04 (m, 8H). ¹⁹F NMR (377 MHz, METHANOL-d4) δ=−76.90 (s, 18F), −108.43 (m, 1F), −112.76-−112.90 (m, 1F).

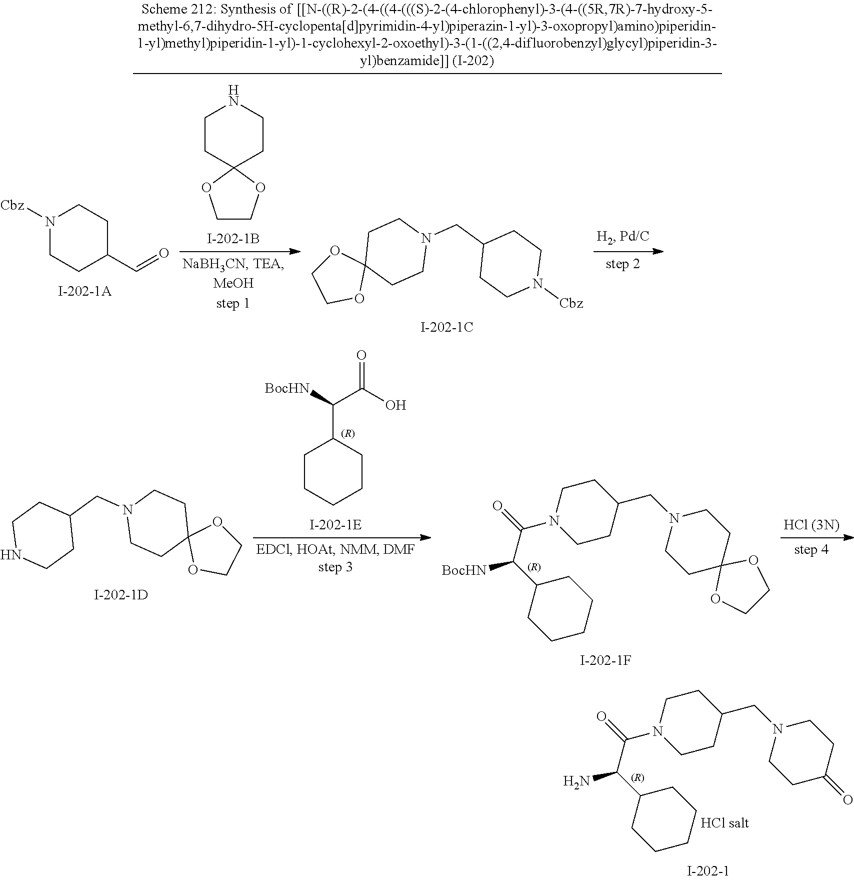

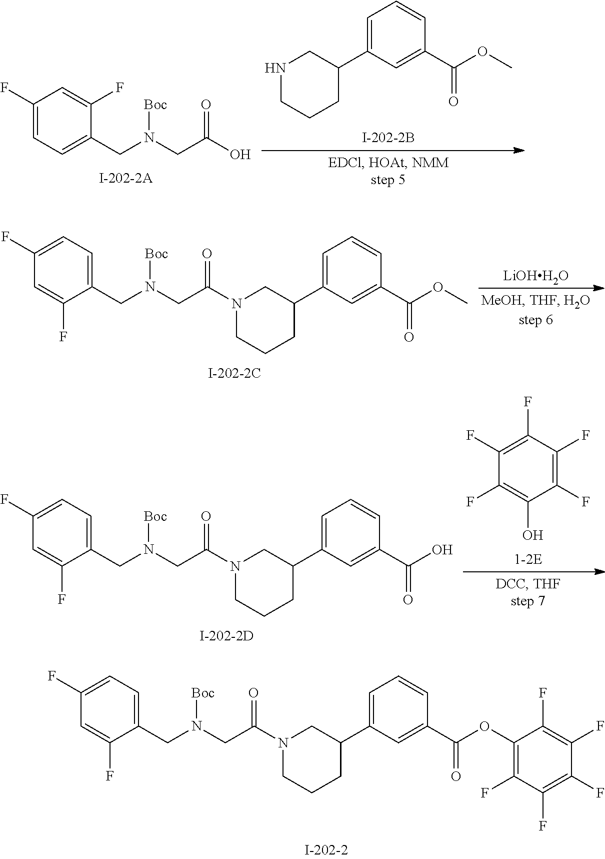

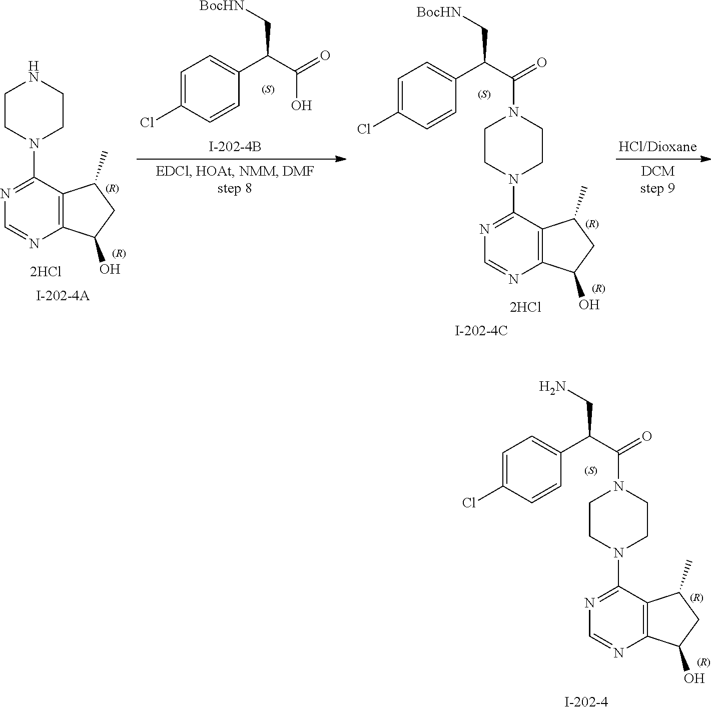

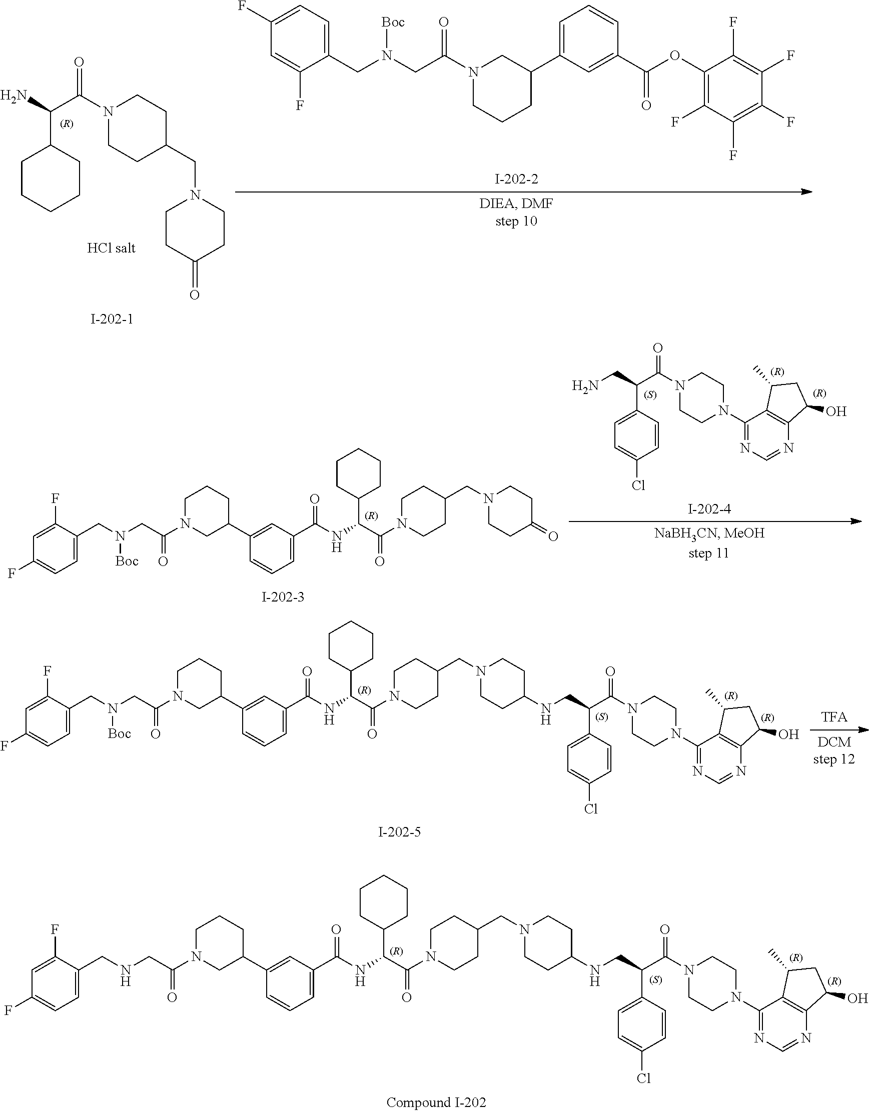

### Step 1: Synthesis of I-202-1C

To a solution of I-202-1B (1.45 g, 8.09 mmol, 1 eq) in MeOH (20 mL) was added TEA (818.39 mg, 8.09 mmol, 1.13 mL, 1 eq). The mixture was stirred at 25° C. for 10 min. Then I-202-1A (2 g, 8.09 mmol, 1 eq) was added. The mixture was stirred at 25° C. for 20 min. Then NaBH₃CN (1.52 g, 24.26 mmol, 3 eq) was added into the mixture. The reaction mixture was then stirred at 25° C. for 1 hr. The mixture was concentrated to get a residue. The residue was purified by reversed phase HPLC (ISCO®; 120 g SepaFlash® C18 Column, Eluent of 0˜30% (0.1% FA) water/MeCN @ 100 mL/min) then lyophilized. I-202-1C (2.3 g, 6.08 mmol, 75.18% yield, 99% purity) as colorless oil. LCMS: R^{t}=0.397 min, [M+H]⁺=375.1. ¹H NMR (400 MHZ, METHANOL-d4) δ=7.52-7.20 (m, 5H), 5.11 (s, 2H), 4.24-4.12 (m, 2H), 3.99 (s, 4H), 3.16 (br s, 4H), 2.94-2.77 (m, 4H), 2.03-1.96 (m, 1H), 1.93 (m, 4H), 1.79 (br d, J=12.8 Hz, 2H), 1.24-1.10 (m, 2H).

### Step 2: Synthesis of I-202-1D

A mixture of Pd/C (0.5 g, 469.84 μmol, 10% purity) in MeOH (10 mL) was added a solution of I-202-1C (2.3 g, 6.14 mmol, 1 eq) in MeOH (13 mL) then degassed and purged with H₂ (15 psi) for 3 times, and then the mixture was stirred at 25° C. for 12 hr under H₂ atmosphere. The reaction mixture was filtered, and concentrated to get product. The product was used as the next step without further purification. I-202-1D (1.3 g, 5.14 mmol, 83.66% yield, 95% purity) as white solid. ¹H NMR (400 MHZ, METHANOL-d4) δ=3.92 (s, 4H), 3.03 (br d, J=12.4 Hz, 2H), 2.59 (m, 2H), 2.50 (br s, 4H), 2.21 (d, J=6.8 Hz, 2H), 1.76 (br d, J=14.4 Hz, 2H), 1.73-1.60 (m, 5H), 1.19-1.05 (m, 2H).

### Step 3: Synthesis of I-202-1F

A mixture of I-202-1D (1.26 g, 5.24 mmol, 1 eq), I-202-1E (1.62 g, 6.29 mmol, 1.2 eq), EDCI (3.02 g, 15.73 mmol, 3 eq), HOAt (1.07 g. 7.86 mmol, 1.10 mL, 1.5 eq) and NMM (3.18 g, 31.46 mmol, 3.46 mL, 6 eq) in DMF (13 mL) was degassed and purged with N₂ for 3 times, and then the mixture was stirred at 25° C. for 1 hr under N₂ atmosphere. The reaction was quenched by water (5 ml) to get a mixture. The mixture was purified by reversed phase HPLC (ISCO®; 120 g SepaFlash® C18 Column, Eluent of 0˜65% (0.1% FA) water/MeCN @ 100 mL/min). The eluent was concentrated to removed MeCN, extracted with (CHCl3/i-PrOH=3/1) (50 mL+3) and the organic layers were concentrated again. I-202-1F (2.4 g. 5.00 mmol, 95.44% yield, 100% purity) as yellow oil. LCMS: R^{t}=0.466 min, [M+H]⁺=480.2. SFC: R^{t}=1.348 min. ¹H NMR (400 MHZ, METHANOL-d4) δ=6.50 (br d, J=8.8 Hz, 1H), 4.58-4.44 (m, 1H), 4.43-4.30 (m, 1H), 4.19-4.06 (m, 1H), 3.94 (s, 4H), 3.21-3.06 (m, 1H), 2.76-2.59 (m, 4H), 2.38 (br s, 2H), 1.98-1.80 (m, 3H), 1.80-1.69 (m, 7H), 1.69-1.54 (m, 3H), 1.43 (d, J=2.8 Hz, 9H), 1.31-1.20 (m, 3H), 1.17-1.14 (m, 2H), 1.13-0.95 (m, 3H).

### Step 4: Synthesis of I-202-1

A mixture of I-202-1F (2 g, 4.17 mmol, 1 eq) and HCl (3 M, 20.00 mL, 14.39 eq) was stirred at 50° C. for 12 hr. The reaction mixture was cooled to 0° C., and quenched by NaHCO₃ (2 g) to get a mixture. The mixture was purified by reversed phase HPLC (ISCO®; 120 g SepaFlash® C18 Column, Eluent of 0˜10% (0.1% HCl) water/MeCN @ 100 mL/min) and the eluent was collected. Then the eluent was lyophilized. I-202-1 (1.7 g, 1.96 mmol, 46.92% yield, 47% purity, 2 HCl) as colorless oil was obtained. LCMS (Method B): R^{t}=0.365 min, [M+H]⁺=336.1, amx. LCMS (Method B): R^{t}=0.367 min, [M+H]⁺=336.1. ¹H NMR (400 MHZ, METHANOL-d4) δ=4.64-4.48 (m, 1H), 4.38-4.24 (m, 1H), 4.07-3.96 (m, 3H), 3.65 (br d, J=12.6 Hz, 1H), 3.58-3.45 (m, 1H), 3.28-3.16 (m, 2H), 3.15-3.03 (m, 3H), 2.87-2.71 (m, 1H), 2.30-2.18 (m, 1H), 2.15 (br d, J=13.6 Hz, 1H), 2.10-1.90 (m, 4H), 1.89-1.63 (m, 6H), 1.44-1.07 (m, 7H).

### Step 5: Synthesis of I-202-2C

To a solution of I-202-2A (5 g, 16.60 mmol, 1 eq), HOAt (3.39 g, 24.89 mmol, 3.48 mL, 1.5 eq), NMM (8.39 g, 82.98 mmol, 9.12 mL, 5 eq) in DCM (60 mL) was added EDCI (4.77 g, 24.89 mmol, 1.5 eq). The mixture was stirred at 25° C. for 0.5 h. Then I-202-2B (4.67 g, 18.26 mmol, 1.1 eq, HCl) was added. The mixture was stirred for another 1 h. The mixture was quenched by saturated aqueous citric acid (200 mL), washed with aq. NaHCO₃, brine, dried over anhydrous Na₂SO₄, filtered, and evaporated. I-202-2C (8.2 g, 15.01 mmol, 90.45% yield, 92% purity) as yellow gum. LCMS: R^{t}=0.642 min, [M+H]⁺=503.1. ¹H NMR (400 MHZ, CHLOROFORM-d) δ=7.99-7.82 (m, 2H), 7.52-7.27 (m, 3H), 6.98-6.55 (m, 2H), 4.88-4.30 (m, 3H), 4.25-3.61 (m, 6H), 3.25-2.93 (m, 1H), 2.84-2.53 (m, 2H), 2.16-2.03 (m, 1H), 1.92-1.81 (m, 1H), 1.81-1.60 (m, 2H), 1.46 (br d, J=7.6 Hz, 9H).

### Step 6: Synthesis of I-202-2D

To a solution of I-202-2C (8.2 g, 16.32 mmol, 1 eq) in MeOH (30 mL) and THF (30 mL) was added a solution of LiOH·H₂O (2.05 g, 48.95 mmol, 3 eq) in H₂O (30 mL). The mixture was stirred at 25° C. for 1 hr. The mixture was evaporated to removed THF and MeOH, then the mixture was acidified by 1N HCl to PH=3˜4 and extracted with EA 200 mL. The EA layer was separated, dried over anhydrous Na₂SO₄, filtered, evaporated to give I-202-2D (8 g, 13.59 mmol, 83.30% yield, 83% purity) as yellow gum oil. LCMS: R^{t}=0.581 min, [M+H]⁺=489.1. ¹H NMR (400 MHZ, CHLOROFORM-d) δ=8.10-7.86 (m, 2H), 7.54-7.30 (m, 3H), 6.92-6.74 (m, 2H), 4.88-4.37 (m, 3H), 4.28-4.15 (m, 1H), 4.07-3.66 (m, 2H), 3.16-2.98 (m, 1H), 2.88-2.50 (m, 2H), 2.12-2.06 (m, 1H), 1.99-1.52 (m, 4H), 1.46 (br d, J=7.6 Hz, 9H).

### Step 7: Synthesis of I-202-2

To a solution of I-202-2D (5 g, 10.23 mmol, 1 eq) and I-202-2E (2.07 g, 11.26 mmol, 1.1 eq) in THF (50 mL) was added DCC (2.32 g, 11.26 mmol, 2.28 mL, 1.1 eq). The mixture was stirred at 25° C. for 1 hr. The mixture was concentrated to get a residue. The residue was purified by flash silica gel chromatography (ISCO®; 80 g SepaFlash® Silica Flash Column, Eluent of 0˜35% Ethyl acetate/Petroleum ether gradient (@ 20 mL/min). The eluent was concentrated to get I-202-2 (5.24 g, 7.60 mmol, 74.30% yield, 95% purity) as colorless gum. LCMS: R^{t}=0.722 min, [M+H]⁺=655.1. ¹H NMR (400 MHZ, CHLOROFORM-d) δ=8.18-7.94 (m, 2H), 7.64-7.47 (m, 2H), 7.47-7.28 (m, 1H), 6.94-6.69 (m, 2H), 4.82-4.66 (m, 1H), 4.66-4.41 (m, 2H), 4.27-4.15 (m, 1H), 4.10-3.70 (m, 2H), 3.09 (br d, J=12.8 Hz, 1H), 2.90-2.52 (m, 2H), 2.20-2.08 (m, 1H), 1.98-1.60 (m, 3H), 1.53-1.39 (m, 9H).

### Step 8: Synthesis of I-202-4C

To a solution of I-202-4B (2 g, 6.67 mmol, 1 eq), I-202-4A (2.05 g, 6.67 mmol, 1 eq, 2HCl), HOAt (1.36 g, 10.01 mmol, 1.40 mL, 1.5 eq), NMM (4.05 g, 40.03 mmol, 4.40 mL, 6 eq) in DMF (30 mL) was added EDCI (1.92 g, 10.01 mmol, 1.5 eq) at 0° C. The mixture was stirred at 25° C. for 1 h. The mixture was quenched with water (60 mL) and extracted with TBME (60 mL*2). The combined organic layers were filtered, and washed with water (60 mL), brine (60 mL), dried over anhydrous Na₂SO₄, filtered. The organic solution was evaporated to afford I-202-4C (3.3 g, 6.26 mmol, 93.78% yield, 97.85% purity) as a yellow solid. LCMS: R^{t}=0.469 min, [M+H]⁺=516.3. ¹H NMR (400 MHZ, CHLOROFORM-d) δ=8.54-8.46 (m, 1H), 7.33 (dd, J=1.6, 8.4 Hz, 2H), 7.22 (br d, J=8.4 Hz, 2H), 5.21-5.05 (m, 2H), 4.12-4.05 (m, 1H), 3.87-3.84 (m, 2H), 3.70-3.35 (m, 8H), 3.25-3.13 (m, 1H), 2.24-2.09 (m, 2H), 1.42 (s, 9H), 1.15-1.13 (m, 3H).

### Step 9: Synthesis of I-202-4

To a solution of I-202-4C (3.3 g, 6.39 mmol, 1 eq) in DCM (20 mL) was added 4N HCl/dioxane (20 mL). The mixture was stirred at 25° C. for 1 h. The mixture was evaporated and then quenched with ammonia to PH>7. Then the residual solution was purified by reversed phase HPLC purification (ammonia condition) to give I-202-4 (1.8 g, 4.22 mmol, 65.98% yield, 97.5% purity) as white foam. LCMS: R^{t}=0.354 min, [M+H]⁺=416.2. HPLC: R^{t}=0.737 min.

### Step 10: Synthesis of I-202-3

To a solution of I-202-1 (1.7 g, 4.16 mmol, 1 eq, 2HCl) in DMF (17 mL) was added I-202-2 (2.72 g. 4.16 mmol, 1 eq) and DIEA (2.69 g, 20.81 mmol, 3.63 mL, 5 eq). The mixture was stirred at 25° C. for 0.5 hr. The reaction was quenched by water (50 mL), extracted with EA (50 mL*3). The combined organic layers were dried over Na₂SO₄ and concentrated to get a residue. The residue was purified by Prep-HPLC (column: Waters Xbridge BEH C18 250*50 mm*10 um; mobile phase: [water (ammonia hydroxide v/v)—ACN]; gradient: 50%-80% B over 20 min). The eluent was concentrated to remove MeCN and extracted with EA (50 mL*3), the combined organic layers were dried over Na₂SO₄ and concentrated to get product. I-202-3 (1.16 g, 1.40 mmol, 33.54% yield, 97% purity) as white solid was obtained. LCMS: R^{t}=0.552 min, [M+H]⁺=806.3. HPLC: R^{t}=13.749 min. SFC: R^{t}=1.335 min, 1.932 min. ¹H NMR (400 MHZ, CHLOROFORM-d) δ=7.76-7.61 (m, 2H), 7.39 (br s, 2H), 7.35-7.28 (m, 1H), 7.12-6.93 (m, 1H), 6.90-6.71 (m, 2H), 5.09 (br d, J=3.6 Hz, 1H), 4.78-4.36 (m, 4H), 4.23-4.14 (m, 1H), 4.11-3.88 (m, 2H), 3.88-3.58 (m, 1H), 3.22-2.96 (m, 2H), 2.73 (m, 5H), 2.69-2.53 (m, 2H), 2.52-2.40 (m, 4H), 2.38-2.23 (m, 2H), 2.09 (br d, J=2.0 Hz, 1H), 2.04-1.61 (m, 11H), 1.46 (br d, J=7.6 Hz, 9H), 1.26-1.01 (m, 7H).

### Step 11: Synthesis of I-202-5

To a solution of I-202-3 (1 g, 1.24 mmol, 1 eq) and I-202-4 (516.03 mg, 1.24 mmol, 1 eq) in MeOH (10 mL) was added NaBH₃CN (233.91 mg, 3.72 mmol, 3 eq). The mixture was stirred at 25° C. for 0.5 hr. The reaction was quenched by water (5 mL), concentrated to remove MeOH, extracted with EA (10 mL*3) and the combined organic layers were concentrated to get a residue. The residue was purified by Prep-HPLC (column: Waters Xbridge Prep OBD C18 150*40 mm*10 um; mobile phase: [water (NH₄HCO₃)-ACN]; gradient: 50%-80% B over 52 min). The eluent was concentrated to remove MeCN, extracted with EA (50 mL*3), the combined organic layers were dried over Na₂SO₄ and concentrated to get product. I-202-5 (900 mg, 716.47 μmol, 57.75% yield, 96% purity) as gray solid was obtained, HPLC ( ), and. 1 LCMS: R^{t}=0.503 min, [M+H]⁺=1206.8. HPLC: R^{t}=17.464 min. SFC: R^{t}=6.405 min, 6.794 min. 1H NMR (400 MHZ, METHANOL-d4) δ=8.41 (s, 1H), 7.82-7.65 (m, 2H), 7.51-7.41 (m, 2H), 7.38 (br d, J=8.0 Hz, 3H), 7.34-7.27 (m, 2H), 7.03-6.87 (m, 2H), 5.04-4.91 (m, 2H), 4.61-4.40 (m, 4H), 4.33-4.11 (m, 4H), 4.07 (s, 1H), 3.95-3.80 (m, 2H), 3.79-3.65 (m, 2H), 3.64-3.42 (m, 4H), 3.30-3.24 (m, 2H), 3.23-3.03 (m, 2H), 2.98-2.83 (m, 2H), 2.82-2.61 (m, 4H), 2.56-2.36 (m, 1H), 2.23 (br d, J=6.4 Hz, 1H), 2.19-2.14 (m, 1H), 2.14-2.09 (m, 1H), 2.07 (m, 2H), 2.01-1.94 (m, 2H), 1.85 (br s, 8H), 1.80-1.73 (m, 2H), 1.69 (br s, 2H), 1.62-1.51 (m, 1H), 1.50-1.37 (m, 10H), 1.37-1.24 (m, 5H), 1.21-1.17 (m, 1H), 1.16-0.98 (m, 6H). ¹⁹F NMR (377 MHz, METHANOL-d4) δ=−113.406, −113.424, −115.480, —115.904.

### Step 12: Synthesis of I-202

To a solution of I-202-5 (900 mg, 746.33 μmol, 1 eq) in DCM (9 mL) was added TFA (4.61 g, 40.39 mmol, 3 mL, 54.11 eq). The mixture was stirred at 25° C. for 1 hr. The reaction mixture was concentrated to get a residue. The residue was purified by reversed phase HPLC (ISCO®; 80 g SepaFlash R C18 Column, Eluent of 0˜90% (0.1% TFA) water/MeCN (@ 100 mL/min). Collected eluent and lyophilized. I-202 (817 mg, 669.10 μmol, 89.65% yield, 99.9% purity, TFA) was obtained as a white solid. LCMS: R^{t}=0.422 min, [M+H]⁺=1105.6. HPLC: R^{t}=8.017 min. SFC: R^{t}=4.114 min, 5.350 min. 1H NMR (400 MHZ, METHANOL-d4) δ=8.57 (s, 1H), 7.84-7.67 (m, 2H), 7.61 (m, 1H), 7.55-7.40 (m, 4H), 7.37 (br d, J=8.0 Hz, 2H), 7.19-7.06 (m, 2H), 5.30 (m, 1H), 4.92 (m, 1H), 4.68-4.47 (m, 3H), 4.42-4.28 (m, 3H), 4.27-4.07 (m, 3H), 3.99-3.81 (m, 4H), 3.81-3.72 (m, 3H), 3.71-3.62 (m, 3H), 3.62-3.36 (m, 3H), 3.29-3.18 (m, 3H), 3.18-2.98 (m, 4H), 2.94-2.65 (m, 3H), 2.56-2.34 (m, 2H), 2.33-2.26 (m, 1H), 2.26-2.03 (m, 5H), 2.03-1.83 (m, 6H), 1.82-1.74 (m, 2H), 1.74-1.55 (m, 3H), 1.50-1.20 (m, 5H), 1.18 (br d, J=7.2 Hz, 3H), 1.11 (brd, J=12.0 Hz, 2H). ¹⁹F NMR (377 MHz, METHANOL-d4) δ=−77.010, −108.376, −112.924.

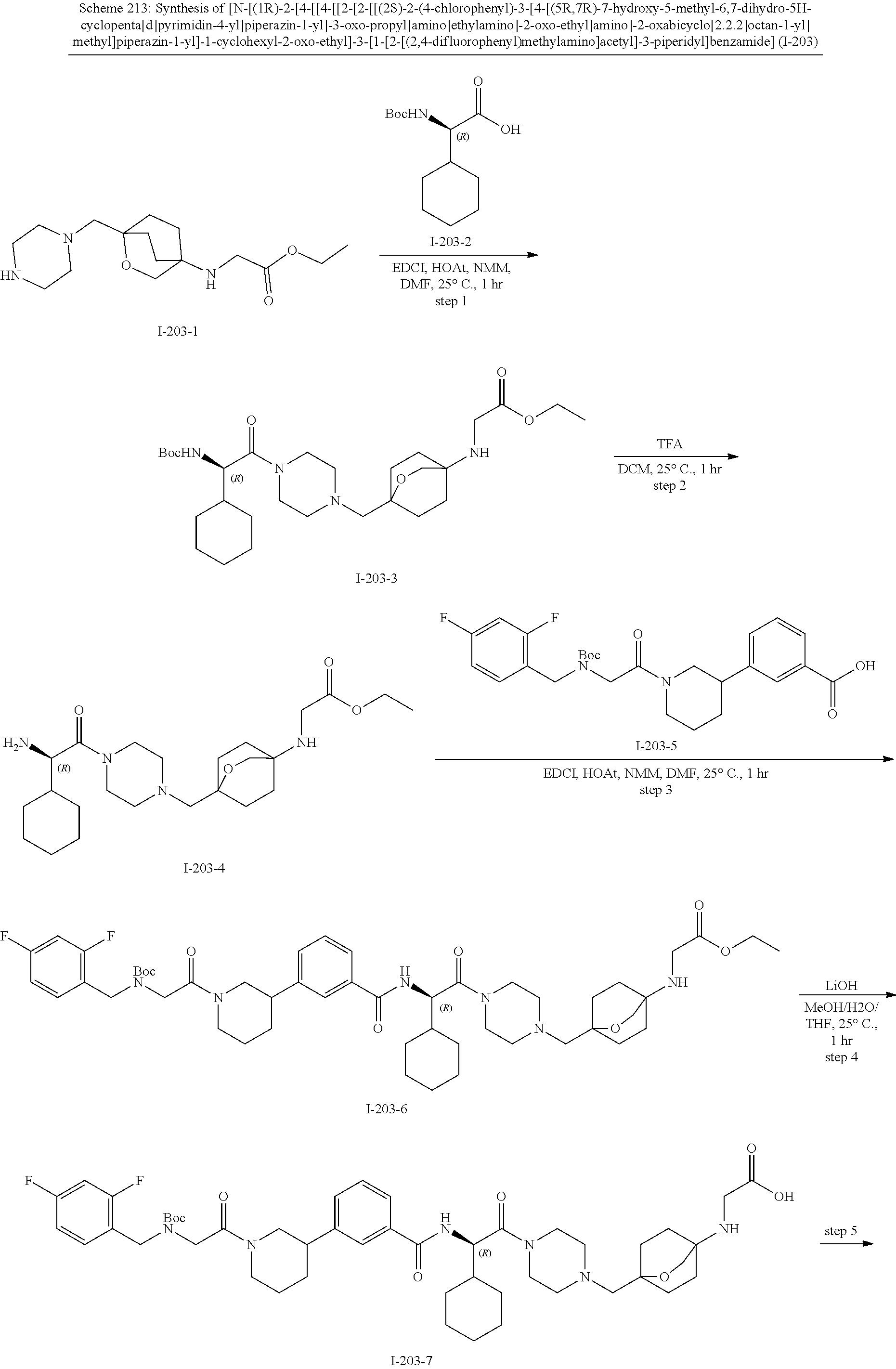

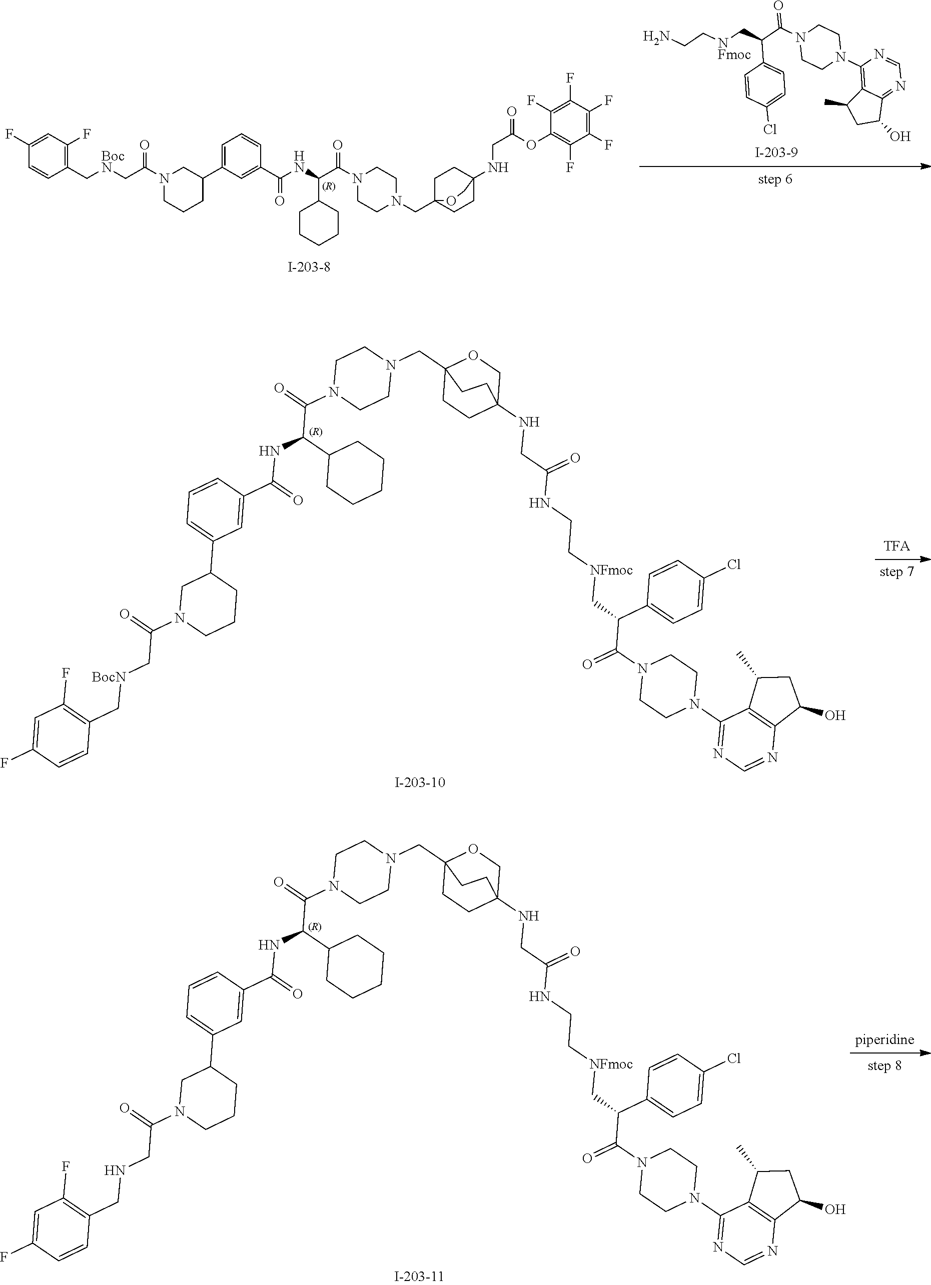

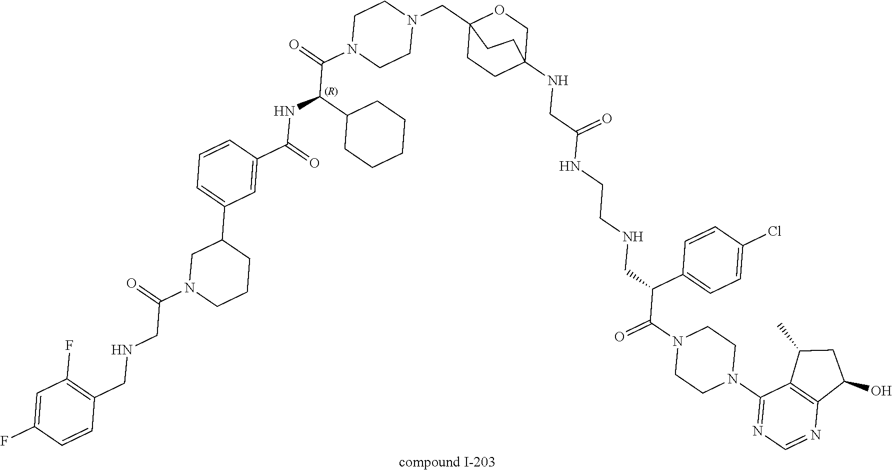

### Step 1: Synthesis of I-203-3

To a solution of I-203-2 (165.26 mg, 642.22 μmol, 1 eq) in DMF (2 mL) was added EDCI (369.34 mg, 1.93 mmol, 3 eq), NMM (324.79 mg, 3.21 mmol, 353.04 μL, 5 eq), I-203-1 (200 mg, 642.22 μmol, 1 eq) and HOAt (87.41 mg, 642.22 μmol, 89.84 μL, 1 eq). The mixture was stirred at 25° C. for 1 h. The crude product was purified by prep-HPLC (0.1% FA condition) and the eluent was lyophilized to give I-203-2 (270 mg, 416.72 μmol, 64.89% yield, 85% purity) as a yellow solid. LCMS (Method D): R^{t}=0.286 min, (M+H)=551.4.

### Step 2: Synthesis of I-203-4

To a solution of I-203-3 (270 mg, 490.26 μmol, 1 eq) in DCM (3 mL) was added TFA (921.00 mg, 8.08 mmol, 0.6 mL, 16.48 eq). The mixture was stirred at 25° C. for 0.5 h. Concentrated under reduced pressure to give I-203-4 (280 mg, crude, TFA) as a yellow oil. LCMS (Method D): R^{t}=0.212 min, (M+H)=451.7.

### Step 3: Synthesis of I-203-6

To a solution of I-203-4 (100 mg, 177.11 μmol, 1 eq, TFA) in DMF (1 mL) was added EDCI (101.85 mg, 531.32 μmol, 3 eq), NMM (89.57 mg, 885.53 μmol, 97.36 μL, 5 eq), I-203-5 (86.52 mg, 177.11 μmol, 1 eq) and HOAt (24.11 mg, 177.11 μmol, 24.77 μL, 1 eq). The mixture was stirred at 25° C. for 1 h. The crude product was purified by prep-HPLC (0.1% FA condition) and the eluent was lyophilized to give I-203-6 (100 mg, 105.31 μmol, 59.46% yield, 97% purity) as a white solid. LCMS (Method D): R^{t}=0.456 min, (M+H)=921.5.

### Step 4: Synthesis of I-203-7

To a solution of I-203-6 (100 mg, 108.56 μmol, 1 eq) in MeOH (0.4 mL), H₂O (0.4 mL) and THF (0.4 mL) was added LiOH·H₂O (13.67 mg, 325.69 μmol, 3 eq). The mixture was stirred at 25° C. for 0.5 h. Concentrated under reduced pressure to give a residue. The crude product was purified by prep-HPLC (0.1% FA condition) and the eluent was lyophilized to give I-203-7 (80 mg, 88.68 μmol, 81.69% yield, 99% purity) as a white solid. LCMS (Method D): R^{t}=0.410 min, (M+H)=893.5.

### Step 5: Synthesis of I-203-8

To a solution of I-203-7 (70 mg, 78.38 μmol, 1 eq) in DMF (0.7 mL) was added N,N′-diisopropylmethanediimine (14.84 mg, 117.57 μmol, 18.21 μL, 1.5 eq) and 2,3,4,5,6-pentafluorophenol (21.64 mg, 117.57 μmol, 1.5 eq). The mixture was stirred at 25° C. for 0.5 h. The reaction mixture was washed with H₂O (2 mL) at 25° C., and mixture was extracted with EA 6 ml (2 ml*3), and combined organic phase was dried with anhydrous sodium sulfate, filtered, and filtrate was concentrated to give I-203-8 (80 mg, crude) as a yellow oil. LCMS (Method D): R^{t}=0.512 min, (M+H)=1059.5.

### Step 6: Synthesis of I-203-10

To a solution of I-203-8 (80 mg, 75.53 μmol, 1 eq) in DMF (0.8 mL) was added DIEA (29.29 mg, 226.60 μmol, 39.47 μL, 3 eq) and I-203-9 (51.46 mg, 75.53 μmol, 1 eq). The mixture was stirred at 25° C. for 0.5 h. The crude product was purified by prep-HPLC (0.1% FA condition) and the eluent was lyophilized to give I-203-10 (60 mg, 21.98 μmol, 29.09% yield, 57% purity) as a white solid. LCMS (Method D): R^{t}=0.503 min, (M/2+H)=778.8.

### Step 7: Synthesis of I-203-11

To a solution of I-203-10 (50 mg, 32.13 μmol, 1 eq) in DCM (0.5 mL) was added TFA (153.50 mg, 1.35 mmol, 100.00 μL, 41.90 eq). The mixture was stirred at 25° C. for 15 min. Concentrated under reduced pressure to give I-203-11 (50 mg, crude, TFA) as a yellow oil. LCMS (Method D): R^{t}=0.512 min, (M/2+H)=728.9.

### Step 8: Synthesis of 1-203

To a solution of I-203-11 (50 mg, 31.84 μmol, 1 eq, TFA) in DMF (0.5 mL) was added piperidine (13.56 mg, 159.22 μmol, 15.72 μL, 5 eq). The mixture was stirred at 25° C. for 15 min. The crude product was purified by prep-HPLC (neutral condition) and the eluent was lyophilized to give I-203 (14.73 mg, 11.68 μmol, 36.69% yield, 97.879% purity) as a white solid. SFC: R^{t}=1532 min, 1.655 min LCMS: R^{t}=0.695 min, (M/2+H)=617.8. ¹H NMR (400 MHZ, METHANOL-d4) δ=8.41 (s, 1H), 7.75 (s, 1H), 7.73-7.67 (m, 1H), 7.50-7.42 (m, 3H), 7.41-7.36 (m, 2H), 7.35-7.31 (m, 2H), 7.00-6.89 (m, 2H), 4.96 (t, J=6.8 Hz, 1H), 4.61-4.52 (m, 1H), 4.21-4.18 (m, 1H), 3.87 (d, J=4.8 Hz, 2H), 3.81 (d, J=10.0 Hz, 3H), 3.78-3.67 (m, 4H), 3.66-3.59 (m, 4H), 3.58-3.50 (m, 5H), 3.49-3.46 (m, 1H), 3.34 (s, 1H), 3.28-3.23 (m, 1H), 3.18 (s, 2H), 3.14-3.05 (m, 1H), 2.83-2.68 (m, 5H), 2.64-2.48 (m, 4H), 2.32-2.24 (m, 2H), 2.19-2.08 (m, 2H), 2.08-1.99 (m, 3H), 1.99-1.91 (m, 2H), 1.87 (d, J=12.0 Hz, 4H), 1.81-1.71 (m, 4H), 1.71-1.58 (m, 7H), 1.38-1.18 (m, 4H), 1.11 (d, J=6.8 Hz, 5H).

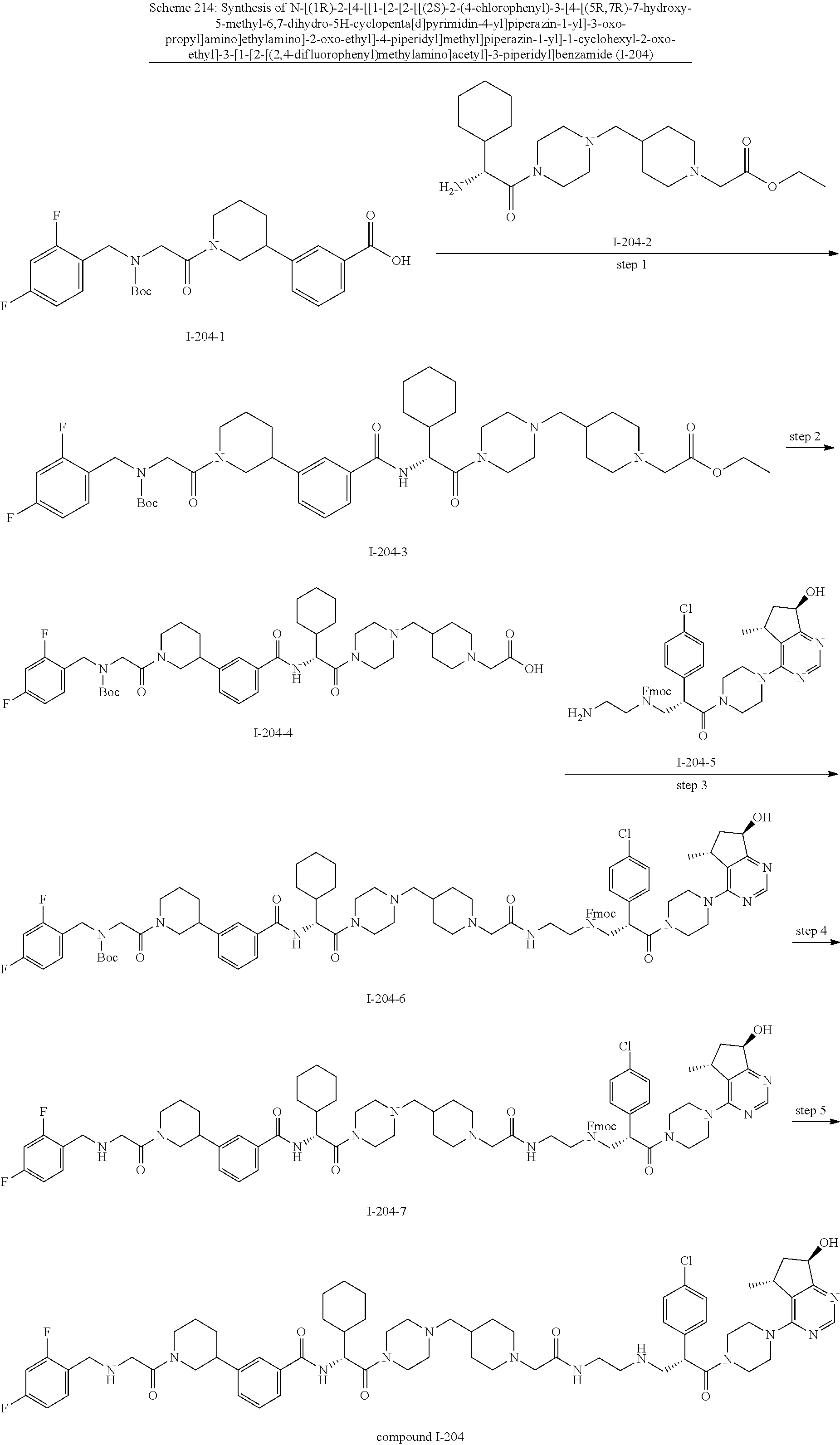

### Step 1: Synthesis of I-204-3

To a solution of I-204-1 (150 mg, 307.05 μmol, 1 eq) in DMF (1.5 mL) was added HOAt (41.79 mg, 307.05 μmol, 42.95 μL, 1 eq) and EDCI (176.58 mg, 921.15 μmol, 3 eq), NMM (155.29 mg, 1.54 mmol, 168.79 μL, 5 eq), I-204-2 (125.45 mg, 307.05 μmol, 1 eq). The mixture was stirred at 25° C. for 1 hr. The reaction mixture was diluted with water (5 mL) and extracted with EA 15 mL (5 mL*3). The combined organic layers were dried over Na₂SO₄, filtered, and concentrated under reduced pressure to give a residue. The residue was purified by prep-HPLC (0.1% FA condition), the eluent was lyophilization to give I-204-3 (110 mg, 125.13 μmol, 40.75% yield) as a white solid. LCMS (Method D): R^{t}=0.404 min, M+H=879.6. SFC: R^{t}=1.987 min, 2.138 min.

### Step 2: Synthesis of I-204-4

To a solution of I-204-3 (100 mg, 113.75 μmol, 1 eq) in THF (0.3 mL), MeOH (0.3 mL), H₂O (0.3 mL) was added LiOH·H₂O (9.55 mg, 227.51 μmol, 2 eq). The mixture was stirred at 25° C. for 1 hr. The mixture was adjusted to pH=5 with CA and extracted with EA 15 mL (5 mL*3). The combined organic layers were dried over Na₂SO₄, filtered, and concentrated under reduced pressure to give I-204-4 (110 mg, crude) as a yellow oil. LCMS (Method D): R^{t}=0.419 min, M+H=851.4. SFC: R^{t}=1.374 min, 1.468 min.

### Step 3: Synthesis of I-204-6

To a solution of I-204-5 (100 mg, 117.50 μmol, 1 eq) in DMF (1 mL) was added HOAt (31.99 mg, 235.01 μmol, 32.87 μL, 2 eq), EDCI (112.63 mg, 587.52 μmol, 5 eq), NMM (118.85 mg, 1.18 mmol, 129.19 μL, 10 eq), I-204-4 (84.33 mg, 117.50 μmol, 1 eq, HCl) was added. The mixture was stirred at 25° C. for 1 hr. The reaction mixture was diluted with water (5 mL) and extracted with EA 15 mL (5 mL*3). The combined organic layers were dried over Na₂SO₄, filtered, and concentrated under reduced pressure to give a residue. The residue was purified by prep-HPLC (0.1% FA condition). The eluent was lyophilization to give I-204-6 (85 mg, 55.48 μmol, 47.22% yield, 98.836% purity) as a yellow oil. LCMS (Method D): R^{t}=0.427 min, M+H=757.8. SFC: R^{t}=2.397 min, 2.944 min.

### Step 4: Synthesis of I-204-7

To a solution of I-204-6 (60 mg, 39.62 μmol, 1 eq) in DCM (0.3 mL) was added TFA (460.50 mg, 4.04 mmol, 0.3 mL, 101.92 eq). The mixture was stirred at 25° C. for 0.5 hr. The reaction mixture was concentrated under reduced pressure to give I-204-7 (65 mg, crude) as a yellow oil. LCMS (Method D): R^{t}=0.400 min, M+H=707.9.

### Step 5: Synthesis of I-204

To a solution of I-204-7 (50 mg, 35.36 μmol, 1 eq) in THF (0.4 mL) was added PIPERIDINE (43.11 mg, 506.30 μmol, 0.05 mL, 14.32 eq). The mixture was stirred at 25° C. for 2 hr. The reaction mixture was concentrated under reduced pressure to give a residue. The residue was purified by prep-HPLC (0.1% Neutral conditional). The eluent was lyophilization to give I-204 (35 mg, 28.45 μmol, 80.47% yield, 96.892% purity) as a white solid. LCMS (Method D): R^{t}=0.289 min, M+H=1191.5. SFC: R^{t}=2.371 min, 2.641 min. ¹H NMR (400 MHZ, METHANOL-d4) δ=8.41 (s, 1H), 7.75 (s, 1H), 7.73-7.68 (m, 1H), 7.49-7.40 (m, 3H), 7.40-7.30 (m, 4H), 6.99-6.89 (m, 2H), 4.96 (t, J=6.8 Hz, 1H), 4.55 (d, J=12.0 Hz, 1H), 4.20-4.17 (m, 1H), 3.89-3.79 (m, 5H), 3.77-3.68 (m, 4H), 3.65-3.46 (m, 9H), 3.17-3.06 (m, 2H), 2.97 (s, 2H), 2.87-2.80 (m, 3H), 2.79-2.72 (m, 4H), 2.54-2.42 (m, 3H), 2.41-2.34 (m, 1H), 2.23 (d, J=7.2 Hz, 2H), 2.16-2.09 (m, 3H), 2.08-2.02 (m, 2H), 1.87 (d, J=13.2 Hz, 4H), 1.76 (d, J=9.6 Hz, 5H), 1.69 (d, J=11.6 Hz, 2H), 1.63-1.53 (m, 2H), 1.35-1.20 (m, 7H), 1.15-1.08 (m, 5H).

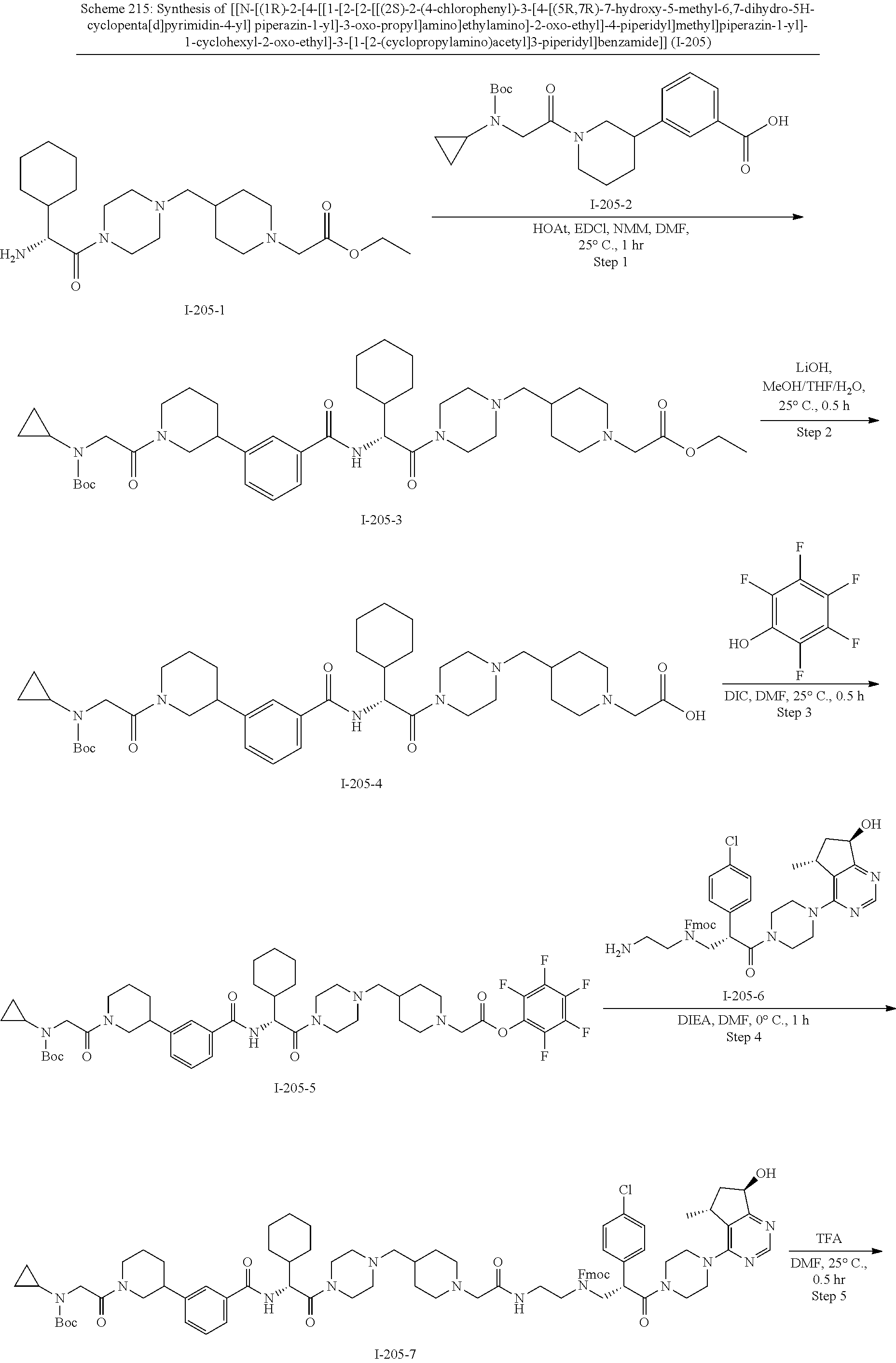

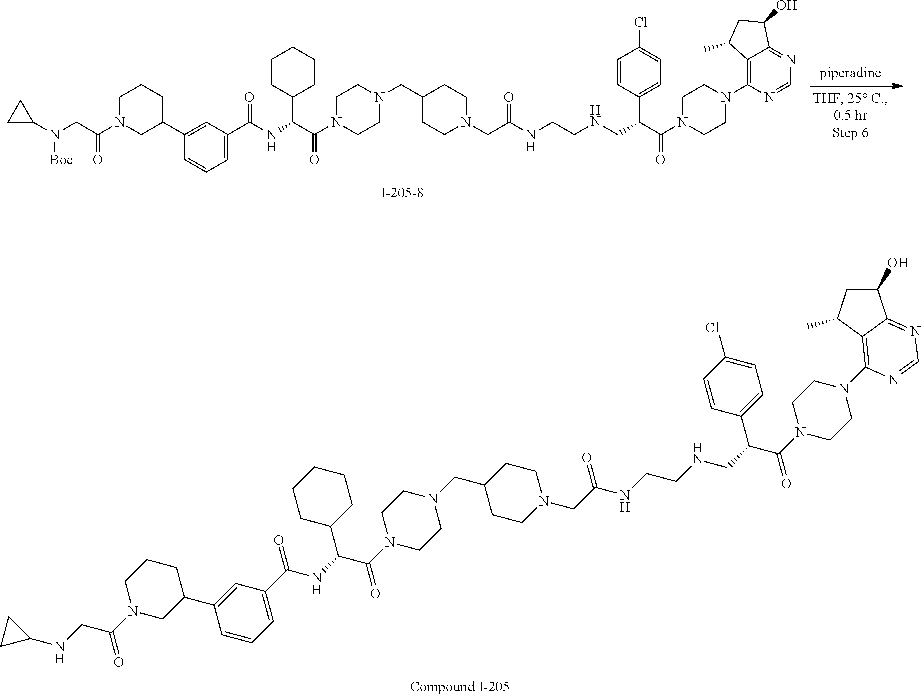 [SMILES: C[C@@H]1C[C@@H](O)c2ncnc(N3CCN(C(=O)[C@H](CNCCNC(=O)CN4CCC(CN5CCN(C(=O)[C@H](NC(=O)c6cccc(C7CCCN(C(=O)CN(C(=O)OC(C)(C)C)C8CC8)C7)c6)C6CCCCC6)CC5)CC4)c4ccc(Cl)cc4)CC3)c21 ; C[C@@H]1C[C@@H](O)c2ncnc(N3CCN(C(=O)[C@H](CNCCNC(=O)CN4CCC(CN5CCN(C(=O)[C@H](NC(=O)c6cccc(C7CCCN(C(=O)CNC8CC8)C7)c6)C6CCCCC6)CC5)CC4)c4ccc(Cl)cc4)CC3)c21]

### Step 1: Synthesis of I-205-3

To a solution of I-205-1 (250 mg, 561.75 μmol, 1 eq, HCl) and I-205-2 (226.09 mg, 561.75 μmol, 1 eq) in DMF (2.5 mL) was added EDCI (323.06 mg, 1.69 mmol, 3 eq), HOAt (76.46 mg, 561.75 μmol, 78.58 μL, 1 eq) and NMM (284.10 mg, 2.81 mmol, 308.80 μL, 5 eq). The mixture was stirred at 25° C. for 1 hr. The reaction mixture was poured into H₂O (2 mL), extract with EA (2 mL*3). The combined organic layers were dried over Na₂SO₄, filtered, and concentrated under reduced pressure to give a residue. The residue was purified by flash silica gel chromatography (ISCO®; 12 g SepaFlash® Silica Flash Column, Eluent of 0˜100% DCM/MeOH @; 36 mL/min, DCM/MeOH=10:1, Rf=0.2) and the eluent was concentrated under reduced pressure to give a product. I-205-3 (500 mg, crude) was obtained as a yellow gum. LCMS (Method D): R^{t}=0.357 min, (M+H)=793.4.

### Step 2: Synthesis of I-205-4

To a solution of ethyl I-205-3 (500 mg, 630.48 μmol, 1 eq) in THF (0.2 mL), MeOH (0.2 mL) and H₂O (0.2 mL) was added LiOH·H₂O (79.37 mg, 1.89 mmol, 3 eq). The mixture was stirred at 25° C. for 0.5 hr. The reaction mixture was poured into H₂O (2 mL) at 25° C., the pH was adjusted to 7 with 1M aqueous HCl, and then extracted with DCM 5 mL*3. The combined organic layers were dried over Na₂SO₄, filtered, and concentrated under reduced pressure to give a residue. The residue was purified by reverse-phase flash (neutral condition) and the eluent was concentrated to remove MeCN and then lyophilization. I-205-4 (200 mg, crude) was obtained as a white solid. LCMS (Method D): R^{t}=0.352 min, (M+H)=765.4.

### Step 3: Synthesis of I-205-5

To a solution of I-205-4 (80 mg, 104.58 μmol, 1 eq) in DMF (0.8 mL) was added N,N′-diisopropylmethanediimine (19.80 mg, 156.86 μmol, 24.29 μL, 1.5 eq) and 2,3,4,5,6-pentafluorophenol (28.87 mg, 156.86 μmol, 1.5 eq). The mixture was stirred at 25° C. for 0.5 hr. The reaction mixture was poured into H₂O (5 mL), extract with EA (5 mL*3). The combined organic layers were dried over Na₂SO₄, filtered, and concentrated under reduced pressure to give I-205-5 (70 mg, 75.18 μmol, 71.89% yield) as a yellow gum. LCMS: R^{t}=0.370 min, (M+H)=931.4.

### Step 4: Synthesis of I-205-7

To a solution of I-205-5 (60 mg, 64.44 μmol, 1 eq) and I-205-6 (46.25 mg, 64.44 μmol, 1 eq, HCl) in DMF (0.6 mL) was added DIEA (16.66 mg, 128.89 μmol, 22.45 μL, 2 eq). The mixture was stirred at 0° C. for 0.5 hr. The reaction mixture was poured into H₂O (2 mL), extract with EA (2 mL*3). The combined organic layers were dried over Na₂SO₄, filtered, and concentrated under reduced pressure to give a residue. The residue was purified by reverse-phase flash (FA condition) and the eluent was concentrated to remove MeCN and then lyophilization. I-205-7 (90 mg, 57.53 μmol, 89.28% yield, 91.3% purity) was obtained as a yellow gum. LCMS: R^{t}=0.411 min, (M+H)=1428.2.

### Step 5: Synthesis of I-205-8

To a solution of I-205-7 (80 mg, 56.01 μmol, 1 eq) in DCM (0.8 mL) was added TFA (614.00 mg, 5.38 mmol, 400.00 μL, 96.13 eq). The mixture was stirred at 25° C. for 0.5 hr. The reaction mixture was concentrated under reduced pressure to give I-205-8 (70 mg, 48.54 μmol, 86.66% yield, TFA) as a yellow gum. LCMS: R^{t}=0.356 min, (M+H)=1327.4.

### Step 6: Synthesis of I-205

To a solution of I-205-8 (70 mg, 48.54 μmol, 1 eq, TFA) in THF (0.7 mL) was added PIPERIDINE (123.52 mg, 1.45 mmol, 143.26 μL, 29.88 eq). The mixture was stirred at 25° C. for 0.5 hr. The reaction mixture was concentrated under reduced pressure to give a residue. The residue was purified by prep-HPLC (column: CD03-Welch Xtimate C18 150*25*5 um; mobile phase: [water (FA)-ACN]; gradient: 0%-30% B over 10 min) and the eluent was concentrated to remove MeCN and then lyophilization. I-205 (7.15 mg, 6.47 μmol, 13.32% yield, 100% purity) was obtained as a white solid. LCMS: R^{t}=0.275 min, (M+H)=1105.4. ¹H NMR (400 MHZ, METHANOL-d4) δ=8.52-8.33 (m, 1H), 7.85-7.65 (m, 2H), 7.60-7.40 (m, 4H), 7.36 (d, J=8.0 Hz, 2H), 5.02-4.92 (m, 3H), 4.63-4.42 (m, 2H), 4.19-3.97 (m, 2H), 3.95-3.84 (m, 2H), 3.83-3.67 (m, 5H), 3.55 (s, 8H), 3.45-3.35 (m, 3H), 3.27-3.14 (m, 6H), 2.98-2.64 (m, 3H), 2.62-2.37 (m, 6H), 2.31 (d, J=6.0 Hz, 2H), 2.16-2.02 (m, 3H), 1.89 (d, J=6.4 Hz, 6H), 1.77 (s, 3H), 1.70 (d, J=10.8 Hz, 3H), 1.47-1.21 (m, 6H), 1.18-1.02 (m, 5H), 0.86-0.77 (m, 3H).

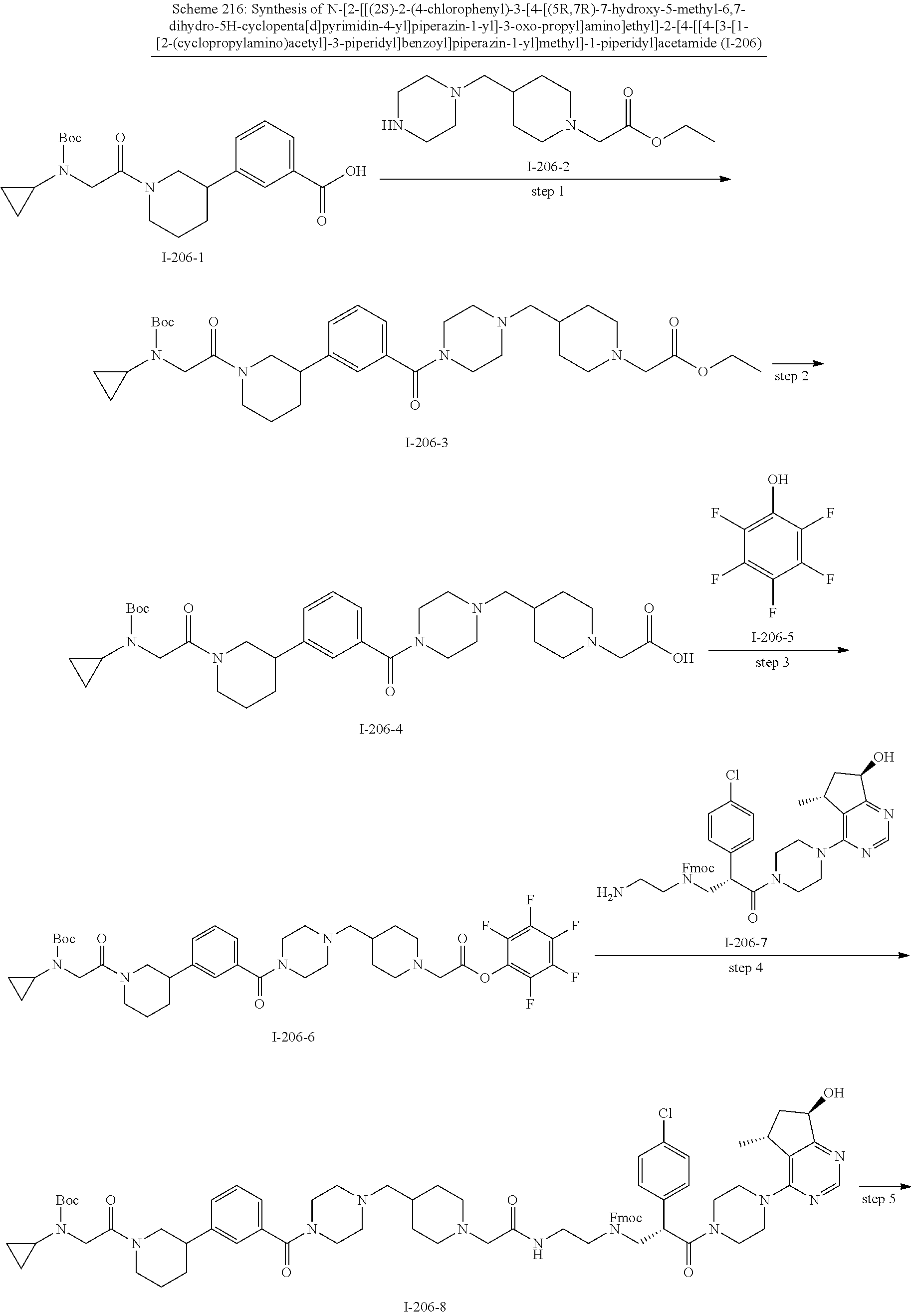

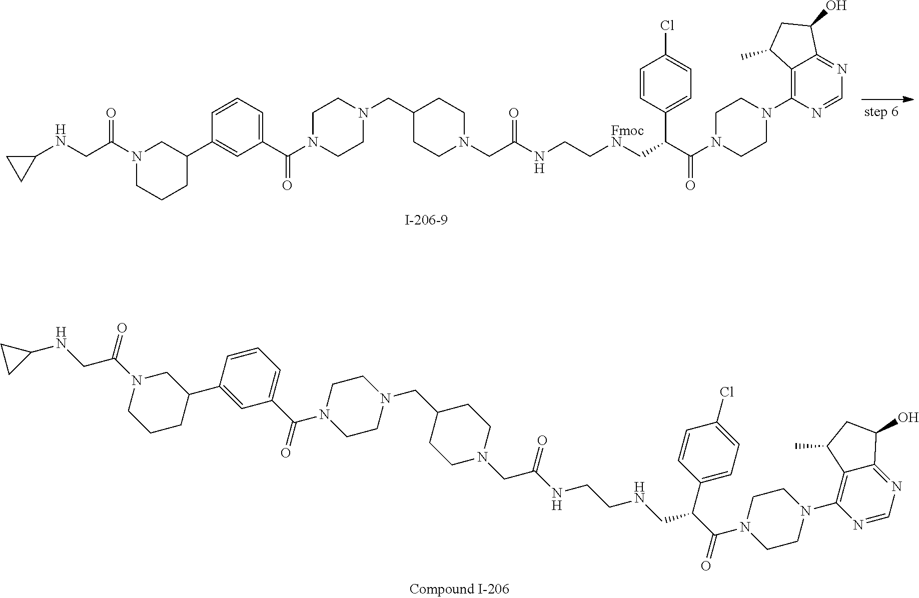

### Step 1: Synthesis of I-206-3

To a solution of I-206-1 (250 mg, 621.14 μmol, 1 eq) in DMF (2.5 mL) was added HOAt (84.54 mg, 621.14 μmol, 86.89 μL, 1 eq), EDCI (357.22 mg, 1.86 mmol, 3 eq), NMM (314.13 mg, 3.11 mmol, 341.45 μL, 5 eq) and I-206-2 (227.97 mg, 745.37 μmol, 1.2 eq, HCl). The mixture was stirred at 25° C. for 1 hr. The residue was diluted with H₂O 30 mL and extracted with EA 90 mL (30 mL*3), dried over Na₂SO₄, filtered, and concentrated under reduced pressure to give a residue. The combined crude product was purified by flash silica gel chromatography (ISCO®; 20 g SepaFlash® Silica Flash Column, Eluent of 0˜100% Petroleum ether/Ethyl acetate @; 60 mL/min). The eluent was concentrated to afford I-206-3 (300 mg, 458.82 μmol, 73.87% yield) as a yellow oil. LCMS (Method D): R^{t}=0.308 min, (M+H)=654.5.

### Step 2: Synthesis of I-206-4

To a solution of I-206-3 (300 mg, 458.82 μmol, 1 eq) in MeOH (1 mL), THF (1 mL) and H₂O (1 mL) was added LiOH·H₂O (57.76 mg, 1.38 mmol, 3 eq). The mixture was stirred at 25° C. for 1 hr. The reaction liquid was concentrated by reducing pressure to obtain the crude product. The crude product was purified by reverse-phase HPLC (Neutral condition), which was concentrated and lyophilizated to afford I-206-4 (200 mg, 319.59 μmol, 69.66% yield) as a yellow solid. LCMS (Method D): R^{t}=0.312 min, (M+H)=626.4.

### Step 3: Synthesis of I-206-6

To a solution of I-206-4 (50 mg, 79.90 μmol, 1 eq) in DMF (0.5 mL) was added I-206-5 (22.06 mg, 119.85 μmol, 1.5 eq) and N,N′-diisopropylmethanediimine (15.12 mg, 119.85 μmol, 18.56 μL, 1.5 eq). The mixture was stirred at 25° C. for 1 hr. The residue was diluted with H₂O 5 mL and extracted with EA 15 mL (5 mL*3), dried over Na₂SO₄, filtered, and concentrated under reduced pressure to give I-206-6 (60 mg, crude) as a yellow oil. LCMS (Method D): R^{t}=0.349 min, (M+Na)=814.6.

### Step 4: Synthesis of I-206-8

To a solution of I-206-6 (60 mg, 75.77 μmol, 1 eq) in DMF (0.6 mL) was added DIEA (19.59 mg, 151.54 μmol, 26.40 μL, 2 eq) and I-206-7 (54.38 mg, 75.77 μmol, 1 eq, HCl). The mixture was stirred at 25° C. for 1 hr. The reaction liquid was concentrated by reducing pressure to obtain the crude product. The crude product was purified by reverse-phase HPLC (0.1% TFA condition), which was concentrated and lyophilizated to afford I-206-8 (40 mg, 27.56 μmol, 36.37% yield, 96.665% purity, TFA) as a white solid. LCMS (Method D): R^{t}=0.383 min, (M+Na)=1310.2. SFC: R^{t}=1.248 min, 1.608 min.

### Step 5: Synthesis of I-206-9

To a solution of I-206-8 (30 mg, 21.38 μmol, 1 eq, TFA) in DCM (0.2 mL) was added TFA (153.50 mg, 1.35 mmol, 0.1 mL, 62.96 eq). The mixture was stirred at 25° C. for 1 hr. The reaction liquid was concentrated by reducing pressure to I-206-9 (30 mg, crude, TFA) as a yellow oil. LCMS (Method D): R^{t}=0.372 min, (M/2)=595.2.

### Step 6: Synthesis of 1-206

To a solution of I-206-9 (30 mg, 23.03 μmol, 1 eq, TFA) in THF (0.3 mL) was added PIPERIDINE (51.73 mg, 607.56 μmol, 0.06 mL, 26.39 eq). The mixture was stirred at 25° C. for 1 hr. The reaction liquid was concentrated by reducing pressure to obtain the crude product. The crude product was purified by reverse-phase HPLC (Neutral condition), which was concentrated and lyophilized to afford I-206 (13.88 mg, 14.36 μmol, 62.36% yield, 100.00% purity) as a white solid. LCMS (Method D): R^{t}=0.220 min, (M+H)=966.4. SFC: R^{t}=2.139 min, 2.378 min. ¹H NMR (400 MHZ, METHANOL-d4) δ=8.41 (s, 1H), 7.46-7.35 (m, 4H), 7.35-7.21 (m, 4H), 4.97 (t, J=6.8 Hz, 1H), 4.61-4.53 (m, 1H), 4.20-4.17 (m, 1H), 3.94-3.84 (m, 2H), 3.81-3.67 (m, 4H), 3.65-3.51 (m, 6H), 3.49-3.38 (m, 3H), 3.38-3.33 (m, 2H), 3.29-3.23 (m, 2H), 3.20-3.09 (m, 1H), 2.97 (s, 2H), 2.90-2.78 (m, 3H), 2.77-2.66 (m, 4H), 2.56-2.34 (m, 4H), 2.28-2.18 (m, 3H), 2.18-2.00 (m, 5H), 1.91-1.72 (m, 4H), 1.67-1.50 (m, 2H), 1.35-1.19 (m, 2H), 1.11 (d, J=7.2 Hz, 3H), 0.56-0.28 (m, 4H).

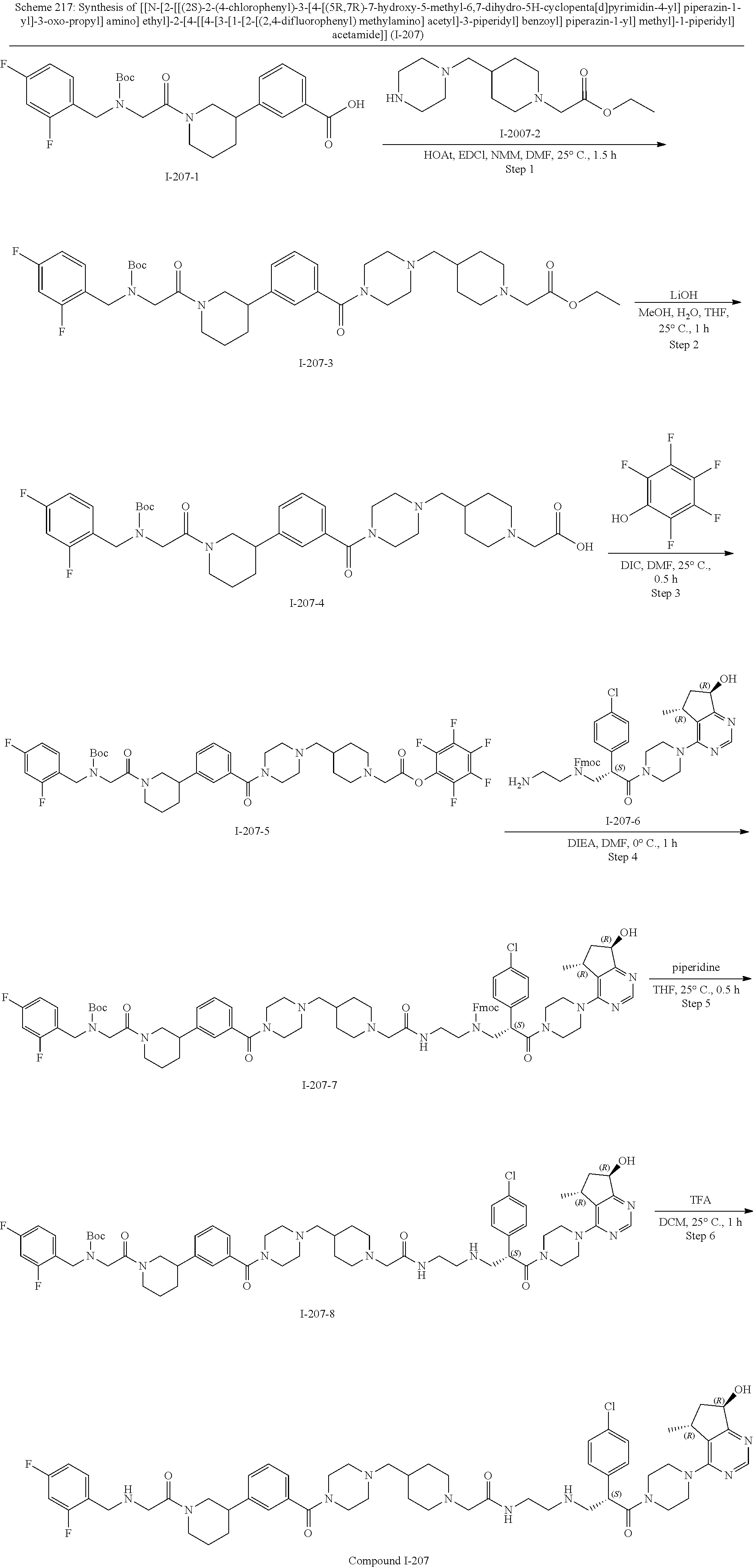

### Step 1: Synthesis of I-207-3

To a solution of I-207-1 (250 mg, 511.75 μmol, 1 eq) and I-207-2 (156.51 mg, 511.75 μmol, 1 eq, HCl) in DMF (3.5 mL) was added EDCI (294.31 mg, 1.54 mmol, 3 eq) HOAt (75.65 mg, 511.75 μmol, 71.59 μL, 1 eq) and NMM (258.81 mg, 2.56 mmol, 281.31 μL, 5 eq). The mixture was stirred at 25° C. for 0.5 hr. The reaction mixture was poured into H₂O (5 mL), extract with EA (5 mL*3). The combined organic layers were dried over Na₂SO₄, filtered, and concentrated under reduced pressure to give a residue. The residue was purified by flash silica gel chromatography (ISCO®; 12 g SepaFlash® Silica Flash Column, Eluent of 0˜100% DCM/MeOH @ 18 mL/min, DCM/MeOH=10:1, Rf=0.3) and the eluent was concentrated under reduced pressure to give I-207-3 (280 mg, crude) as a yellow gum. LCMS (Method D): R^{t}=0.327 min, (M+H)=740.4.

### Step 2: Synthesis of I-207-4

To a solution of I-207-3 (312 mg, 421.68 μmol, 1 eq) in THF (1 mL), MeOH (1 mL) and H₂O (1 mL) was added LiOH·H₂O (53.09 mg, 1.27 mmol, 3 eq). The mixture was stirred at 25° C. for 1 hr. The reaction mixture was poured into H₂O (10 mL) at 25° C., the pH was adjusted to 7 with 1M aqueous HCl, and then extracted with DCM 2 mL*3. The combined organic layers were dried over Na₂SO₄, filtered, and concentrated under reduced pressure to give I-207-4 (400 mg, crude) as a yellow gum. LCMS (Method D): R^{t}=0.335 min, (M+H)=712.3.

### Step 3: Synthesis of I-207-5

To a solution of I-207-4 (56 mg, 78.67 μmol, 1 eq) in DMF (0.5 mL) was added N, N′-diisopropylmethanediimine (14.89 mg, 118.00 μmol, 18.27 μL, 1.5 eq) and 2,3,4,5,6-pentafluorophenol (21.72 mg, 118.00 μmol, 1.5 eq). The mixture was stirred at 25° C. for 0.5 hr. The reaction mixture was poured into H₂O (2 mL), extract with EA (2 mL*3). The combined organic layers were dried over Na₂SO₄, filtered, and concentrated under reduced pressure to give I-207-5 (65 mg, 74.04 μmol, 94.12% yield) as a yellow gum. LCMS: R^{t}=0.355 min, (M+H)=878.3.

### Step 4: Synthesis of I-207-7

To a solution of I-207-5 (65 mg, 74.04 μmol, 1 eq) and I-207-6 (53.14 mg, 74.04 μmol, 1 eq, HCl) in DMF (0.5 mL) was added DIEA (19.14 mg, 148.08 μmol, 25.79 μL, 2 eq). The mixture was stirred at 0° C. for 1 hr. The reaction mixture was poured into H₂O (1 mL), extract with EA (1 mL*3). The combined organic layers were dried over Na₂SO₄, filtered, and concentrated under reduced pressure to give a residue. The residue was purified by reverse-phase flash (FA condition) and the eluent was concentrated to remove MeCN and then lyophilization. I-207-7 (50 mg, crude) was obtained as a yellow solid. LCMS: R^{t}=0.384 min, (M+H)=1374.6.

### Step 5: Synthesis of I-207-8

To a solution of I-207-7 (48 mg, 34.91 μmol, 1 eq) in THF (0.5 mL) was added PIPERIDINE (86.22 mg, 1.01 mmol, 0.1 mL, 29.01 eq). The mixture was stirred at 25° C. for 0.5 hr. The reaction mixture was concentrated under reduced pressure to give I-207-8 (30 mg, 26.02 μmol, 74.55% yield) as a yellow gum.

LCMS: R^{t}=0.302 min, (M+H)=1152.5.

### Step 6: Synthesis of I-207

To a solution of I-207-8 (25 mg, 21.69 μmol, 1 eq) in DCM (0.25 mL) was added TFA (191.88 mg, 1.68 mmol, 125.00 μL, 77.60 eq). The mixture was stirred at 25° C. for 0.5 hr. The reaction mixture was concentrated under reduced pressure to give a residue. The residue was purified by prep-HPLC (column: CD02-Waters Xbidge BEH C18 150*25*10 um; mobile phase: [water (NH4HCO3)-ACN]; gradient: 35%-65% B over 10 min) and the eluent was concentrated to remove MeCN and then lyophilization. I-207 (2.95 mg, 2.49 μmol, 11.47% yield, 98.4% purity, TFA) was obtained as a white solid. LCMS: R^{t}=0.256 min, (M+H)=1052.4. ¹H NMR (400 MHZ, METHANOL-d4) δ=8.41 (s, 1H), 7.56-7.46 (m, 1H), 7.45-7.39 (m, 4H), 7.34 (d, J=8.8 Hz, 3H), 7.28 (d, J=6.0 Hz, 1H), 7.05-6.92 (m, 2H), 4.97 (t, J=6.4 Hz, 1H), 4.57-4.51 (m, 1H), 4.33-4.31 (m, 1H), 3.95 (d, J=9.2 Hz, 2H), 3.91-3.86 (m, 1H), 3.82 (d, J=2.4 Hz, 1H), 3.81-3.75 (m, 3H), 3.70 (d, J=6.4 Hz, 2H), 3.64-3.57 (m, 2H), 3.55-3.51 (m, 2H), 3.49-3.41 (m, 7H), 3.16 (s, 2H), 3.12 (s, 3H), 3.09-3.02 (m, 2H), 3.01-2.94 (m, 4H), 2.80-2.69 (m, 2H), 2.56-2.48 (m, 2H), 2.44-2.37 (m, 2H), 2.26 (d, J=6.4 Hz, 4H), 2.14-2.02 (m, 3H), 1.87-1.76 (m, 4H), 1.67-1.57 (m, 2H), 1.36-1.27 (m, 2H), 1.11 (d, J=6.8 Hz, 3H). F NMR (400 MHz, METHANOL-d4) δ=−76.911, −112.575, −115.697.

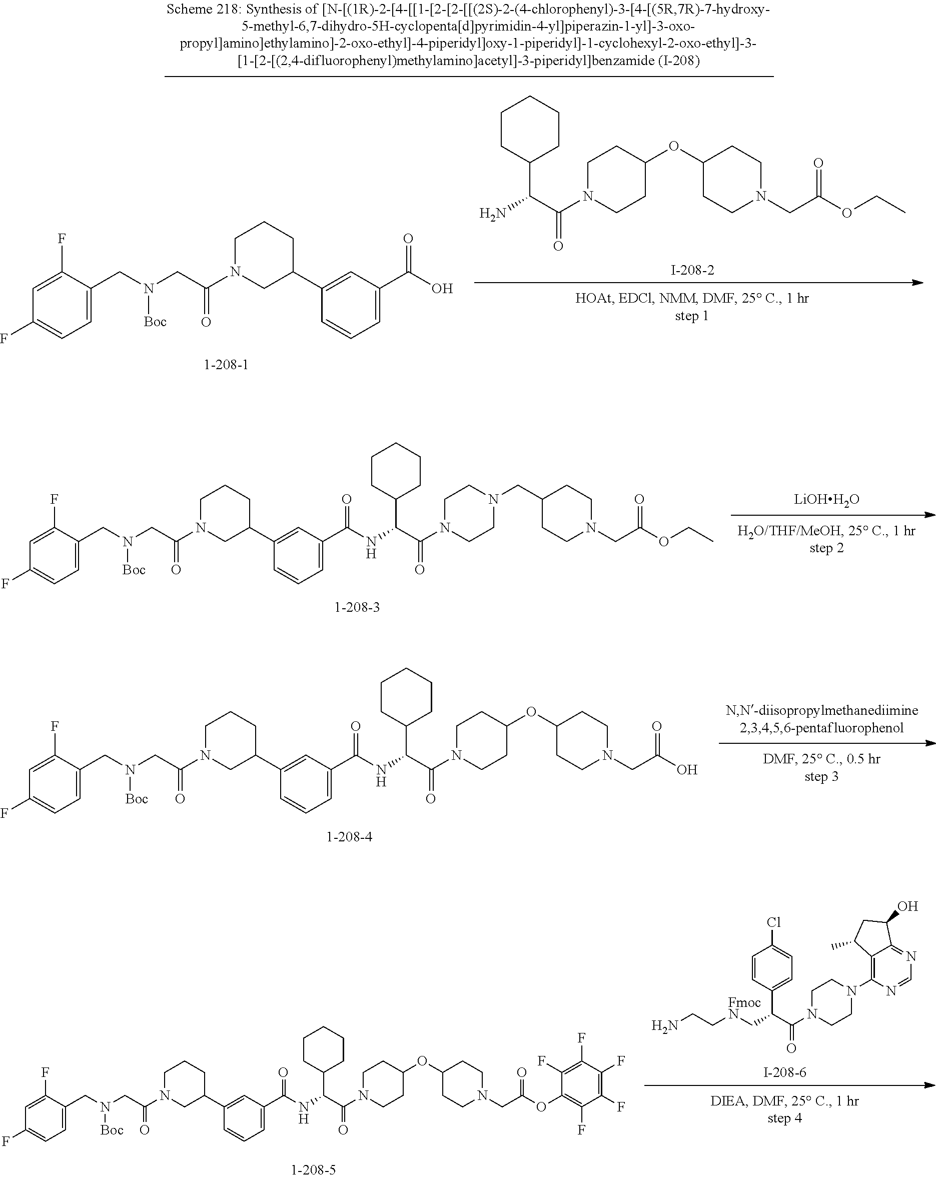

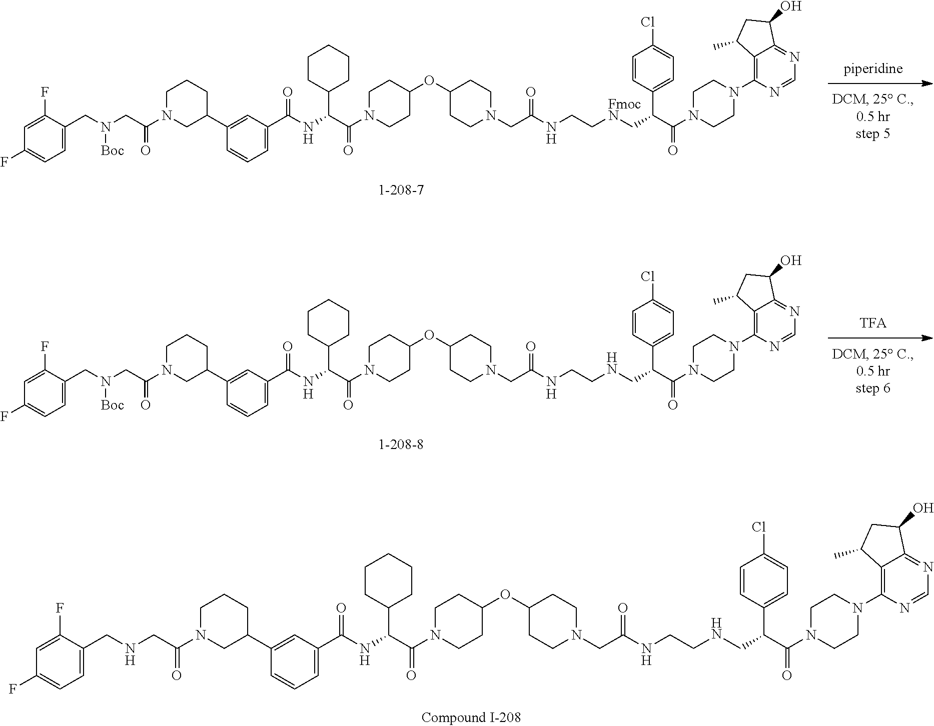

### Step 1: Synthesis of I-208-3.

To a solution of I-208-1 (200 mg, 409.40 μmol, 1 eq) in DMF (2 mL) was added I-208-2 (182.60 mg, 409.40 μmol, 1 eq, HCl), EDCI (235.45 mg, 1.23 mmol, 3 eq), HOAt (55.72 mg, 409.40 μmol, 57.27 μL, 1 eq) and NMM (207.05 mg, 2.05 mmol, 225.05 μL, 5 eq). The mixture was stirred at 25° C. for 2 hr. The reaction mixture was concentrated to give a crude product. The crude product was purified by reverse-phase HPLC (0.1% FA condition) and lyophilized to give I-208-3 (260 mg, 295.76 μmol, 72.24% yield, 100% purity) as a yellow solid. LCMS (Method D): R^{t}=0.459 min, (M+H)=880.5. SFC: R^{t}=1.024 min, 1.272 min.

### Step 2: Synthesis of I-208-4.

To a solution of I-208-3 (250 mg, 284.39 μmol, 1 eq) in H₂O (0.8 mL) and THF (0.8 mL) and MeOH (0.8 mL) was added LiOH. H₂O (35.80 mg, 853.16 μmol, 3 eq). The mixture was stirred at 25° C. for 1 hr. The mixture was adjusted the pH to acid with 1M HCl and quenched by addition H₂O 2.4 mL at 25° C., and extracted with DCM 2.4 mL (0.8 mL*3), dried over Na₂SO₄, filtered, and concentrated under reduced pressure to give I-208-4 (230 mg, crude) as a yellow solid. LCMS (Method D): R^{t}=0.431 min, (M+H)=852.4.

### Step 3: Synthesis of I-208-5.

To a solution of I-208-4 (170 mg, 199.53 μmol, 1 eq) in DMF (2 mL) was added N,N′-diisopropylmethanediimine (37.77 mg, 299.29 μmol, 46.34 μL, 1.5 eq) and 2,3,4,5,6-pentafluorophenol (55.09 mg, 299.29 μmol, 1.5 eq). The mixture was stirred at 25° C. for 0.5 hr. The reaction mixture was quenched by addition H₂O 2 mL at 25° C., and extracted with DCM 1.8 mL (0.6 mL*3), dried over Na₂SO₄, filtered, and concentrated under reduced pressure to give I-208-5 (170 mg, crude) as a yellow oil. LCMS (Method D): R^{t}=0.483 min, (M+H)=1018.3.

### Step 4: Synthesis of I-208-7.

To a solution of I-208-5 (170 mg, 166.98 μmol, 1 eq) and I-208-6 (119.84 mg, 166.98 μmol, 1 eq, HCl) in DMF (2 mL) was added DIEA (32.37 mg, 250.48 μmol, 43.63 μL, 1.5 eq). The mixture was stirred at 25° C. for 1 hr. The reaction mixture was quenched by addition H₂O 2 mL at 25° C., and extracted with EA 2.1 mL (0.7 mL*3), dried over Na₂SO₄, filtered, and concentrated under reduced pressure to give a crude product. The crude product was purified by reverse-phase HPLC (0.1% FA condition) and lyophilized to give I-208-7 (150 mg, 95.74 μmol, 57.34% yield, 96.714% purity) as a yellow oil. LCMS (Method D): R^{t}=0.465 min, (M+H)/2=758.4.

### Step 5: Synthesis of I-208-8.

To a solution of I-208-7 (80 mg, 52.80 μmol, 1 eq) in THF (0.8 mL) was added piperidine (172.44 mg, 2.03 mmol, 0.2 mL, 38.36 eq). The mixture was stirred at 25° C. for 0.5 hr. The mixture was concentrated under reduced pressure to give I-208-8 (80 mg, crude) as a yellow solid. LCMS (Method D): R^{t}=0.459 min, (M+H)/2=647.4.

### Step 6: Synthesis of I-208

To a solution of I-208-8 (80 mg, 61.87 μmol, 1 eq) in DCM (1.0 mL) was added TFA (307.00 mg, 2.69 mmol, 0.2 mL). The mixture was stirred at 25° C. for 0.5 hr. The mixture was dried with N₂ to give a crude product. The crude product was purified by prep-HPLC (column: O-PhenomenexC18 150*10 mm*5 um; mobile phase: [water (TFA)-ACN]; gradient: 15%-35% B over 15 min) and reverse-phase HPLC (0.1% NH₃·H₂O condition) lyophilized to give I-208 (13.63 mg, 10.93 μmol, 17.67% yield, 95.689% purity) as a white solid. LCMS (Method D): R^{t}=0.328 min, (M+H)=1192.4. SFC: R^{t}=1.673 min, 1.804 min. ¹H NMR (400 MHZ, METHANOL-d4) δ=8.41 (d, J=4.4 Hz, 1H), 7.76 (s, 1H), 7.73-7.68 (m, 1H), 7.50-7.41 (m, 3H), 7.41-7.36 (m, 2H), 7.35-7.30 (m, 2H), 6.99-6.90 (m, 2H), 5.00-4.92 (m, 2H), 4.59-4.50 (m, 1H), 4.22-4.18 (m, 1H), 4.09-3.99 (m, 1H), 3.92-3.85 (m, 2H), 3.84-3.81 (m, 2H), 3.79-3.73 (m, 2H), 3.72-3.68 (m, 1H), 3.65-3.56 (m, 3H), 3.56-3.51 (m, 4H), 3.49-3.44 (m, 2H), 3.39-3.34 (m, 2H), 3.27-3.22 (m, 2H), 3.17-3.11 (m, 1H), 2.98 (d, J=6.4 Hz, 2H), 2.83-2.70 (m, 7H), 2.33-2.25 (m, 2H), 2.16-2.09 (m, 1H), 2.06-2.02 (m, 2H), 1.94-1.82 (m, 8H), 1.78 (d, J=6.0 Hz, 2H), 1.72-1.57 (m, 7H), 1.34-1.24 (m, 4H), 1.24-1.19 (m, 1H), 1.16-1.06 (m, 5H), ¹⁹F NMR (376 MHz, METHANOL-d4) δ=−113.244, −116.148.

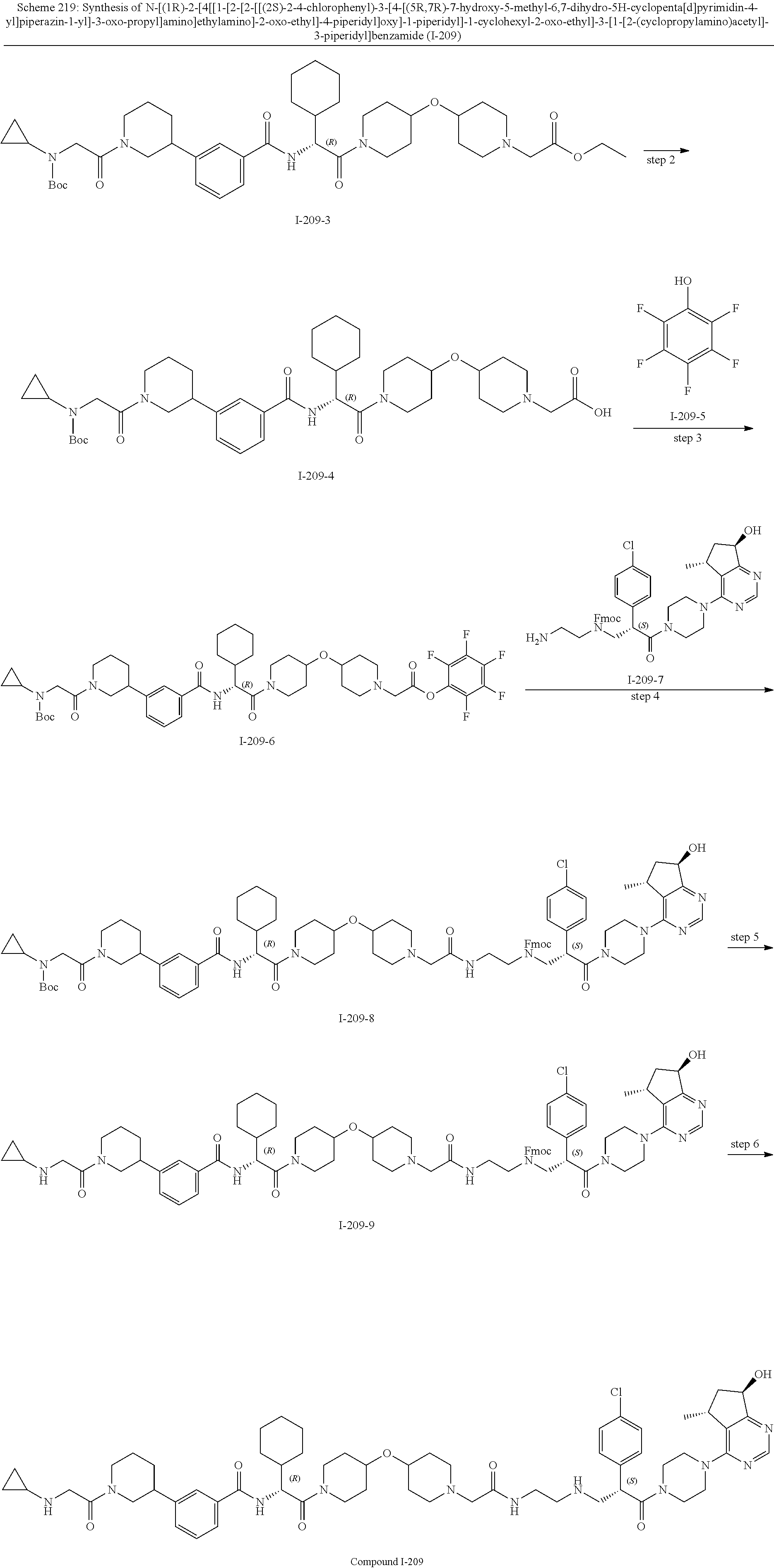

### Step 1: Synthesis of I-209-3

To a solution of I-209-2 (150 mg, 372.69 μmol, 1 eq) in DMF (1.5 mL) was added HOAt (50.73 mg, 372.69 μmol, 52.13 μL, 1 eq), EDCI (214.33 mg, 1.12 mmol, 3 eq), NMM (188.48 mg, 1.86 mmol, 204.87 μL, 5 eq) and I-209-1 (166.23 mg, 372.69 μmol, 1 eq, HCl). The mixture was stirred at 25° C. for 1 hr. The residue was diluted with H₂O 30 mL and extracted with EA 90 mL (30 mL*3), dried over Na₂SO₄, filtered, and concentrated under reduced pressure to give a residue. The combined crude product was purified by flash silica gel chromatography (ISCO®; 12 g SepaFlash® Silica Flash Column, Eluent of 0˜100% Petroleum ether/Ethyl acetate @ 40 mL/min). The eluent was concentrated to afford I-209-3 (200 mg, 229.21 μmol, 61.50% yield, 91% purity) as a yellow oil. LCMS (Method D): R^{t}=0.440 min, (M+H)=794.5.

### Step 2: Synthesis of I-209-4

To a solution of I-209-3 (180 mg, 226.69 μmol, 1 eq) in MeOH (0.6 mL), THF (0.6 mL) and H₂O (0.6 mL) was added LiOH·H₂O (19.03 mg, 453.38 μmol, 2 eq). The mixture was stirred at 25° C. for 1 hr. The mixture was adjusted to pH=5 with citric acid CA and extracted with EA (5 mL*3), dried over Na₂SO₄, filtered, and concentrated under reduced pressure to give a residue. The residue was purified by flash silica gel chromatography (ISCO®; 4 g SepaFlash® Silica Flash Column, Eluent of 0˜100% Dichloromethane/Methanol @ 30 mL/min). The eluent was concentrated under reduced pressure to give I-209-4 (140 mg, 182.77 μmol, 80.63% yield, 100.00% purity) as a yellow oil. LCMS (Method D): R^{t}=0.451 min, (M+H)=766.5.

### Step 3: Synthesis of I-209-6

To a solution of I-209-4 (50 mg, 65.28 μmol, 1 eq) in DMF (0.5 mL) was added N,N′-diisopropylmethanediimine (12.36 mg, 97.91 μmol, 15.16 μL, 1.5 eq) and I-209-5 (18.02 mg, 97.91 μmol, 1.5 eq). The mixture was stirred at 25° C. for 1 hr. The residue was diluted with H₂O 5 mL and extracted with EA 15 mL (5 mL*3), dried over Na₂SO₄, filtered, and concentrated under reduced pressure to give I-209-6 (60 mg, crude) as a yellow oil. LCMS (Method D): R^{t}=0.433 min, (M+H)=932.4.

### Step 4: Synthesis of I-209-8

To a solution of I-209-6 (60 mg, 64.38 μmol, 1 eq) in DMF (0.6 mL) was added DIEA (16.64 mg, 128.75 μmol, 22.43 μL, 2 eq) and I-209-7 (46.20 mg, 64.38 μmol, 1 eq, HCl). The mixture was stirred at 25° C. for 1 hr. The reaction liquid was concentrated by reducing pressure to obtain the crude product. The crude product was purified by reverse-phase HPLC (0.1% TFA condition), which was concentrated and lyophilized to afford I-209-8 (40 mg, 25.92 μmol, 40.26% yield, 100.00% purity, TFA) as a white solid. LCMS (Method D): R^{t}=0.450 min, (M+Na)=1450.9. SFC: R^{t}=2.859 min, 3.222 min.

### Step 5: Synthesis of I-209-9

To a solution of I-209-8 (30 mg, 19.44 μmol, 1 eq, TFA) in DCM (0.2 mL) was added TFA (153.50 mg, 1.35 mmol, 0.1 mL, 69.25 eq). The mixture was stirred at 25° C. for 1 hr. The reaction liquid was concentrated by reducing pressure to give I-209-9 (30 mg, crude, TFA) as a yellow oil. LCMS (Method D): R^{t}=0375 min, (M+H)=1328.5.

### Step 6: Synthesis of I-209

To a solution of I-209-9 (30 mg, 20.79 μmol, 1 eq, TFA) in THF (0.3 mL) was added piperidine (51.73 mg, 607.56 μmol, 0.06 mL, 29.23 eq). The mixture was stirred at 25° C. for 1 hr. The reaction liquid was concentrated by reducing pressure to obtain the crude product. The crude product was purified by reverse-phase HPLC (Neutral condition), which was concentrated and lyophilized to afford I-209 (10.95 mg, 9.89 μmol, 47.59% yield, 100.00% purity) as a white solid. LCMS (Method D): R^{t}=0.305 min, (M+H)=1106.3. ¹H NMR (400 MHz, METHANOL-d4) δ=8.41 (d, J=4.0 Hz, 1H), 7.77 (s, 1H), 7.70 (d, J=3.6 Hz, 1H), 7.53-7.40 (m, 2H), 7.40-7.35 (m, 2H), 7.35-7.29 (m, 2H), 5.01-4.90 (m, 2H), 4.71-4.48 (m, 2H), 4.21-4.15 (m, 1H), 4.10-4.00 (m, 1H), 3.96-3.82 (m, 3H), 3.81-3.68 (m, 3H), 3.65-3.59 (m, 2H), 3.58-3.51 (m, 5H), 3.50-3.43 (m, 2H), 3.26-3.11 (m, 4H), 3.05-2.93 (m, 3H), 2.74 (d, J=3.6 Hz, 6H), 2.34-2.20 (m, 3H), 2.14-2.01 (m, 3H), 1.94-1.83 (m, 7H), 1.80-1.74 (m, 2H), 1.72-1.45 (m, 9H), 1.33-1.20 (m, 3H), 1.17-1.05 (m, 5H), 0.50-0.34 (m, 4H).

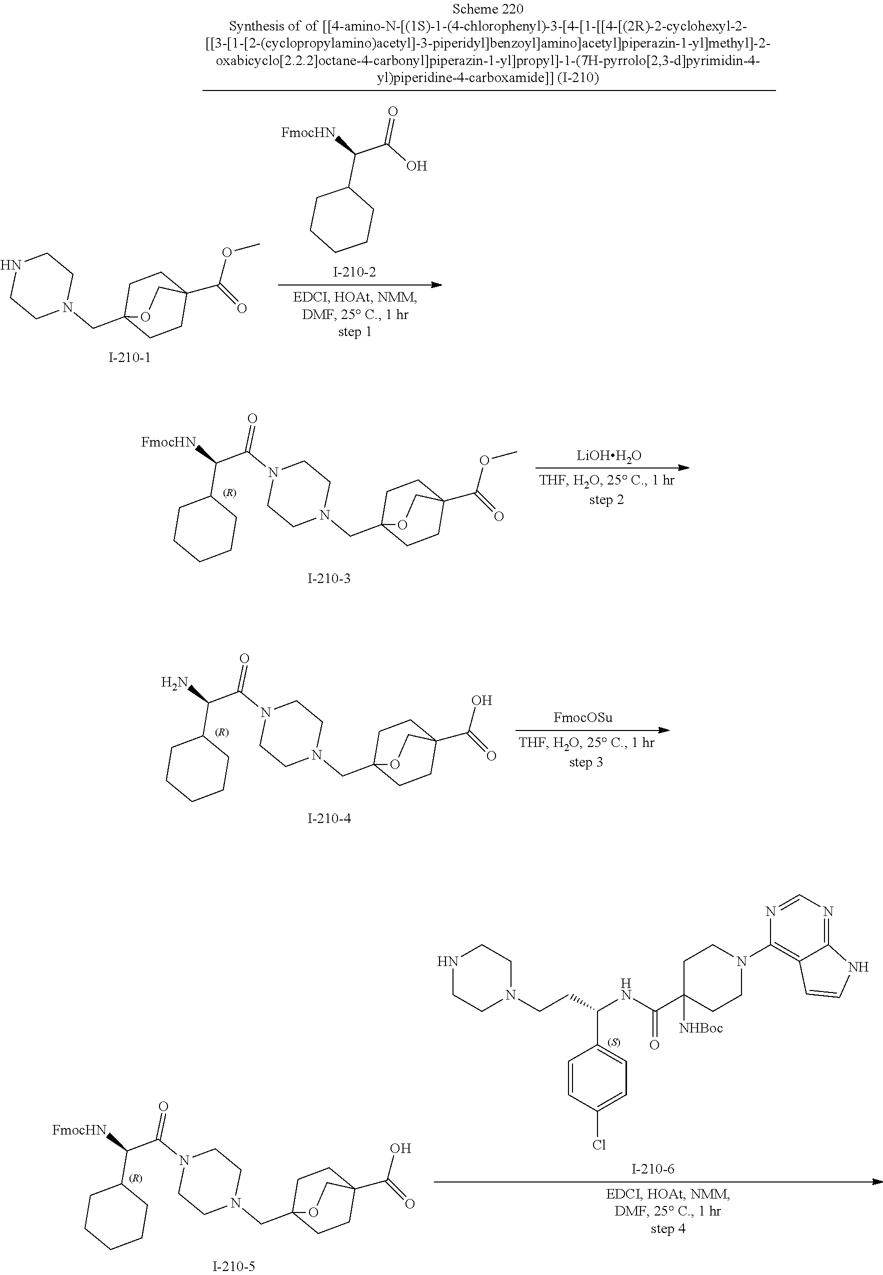

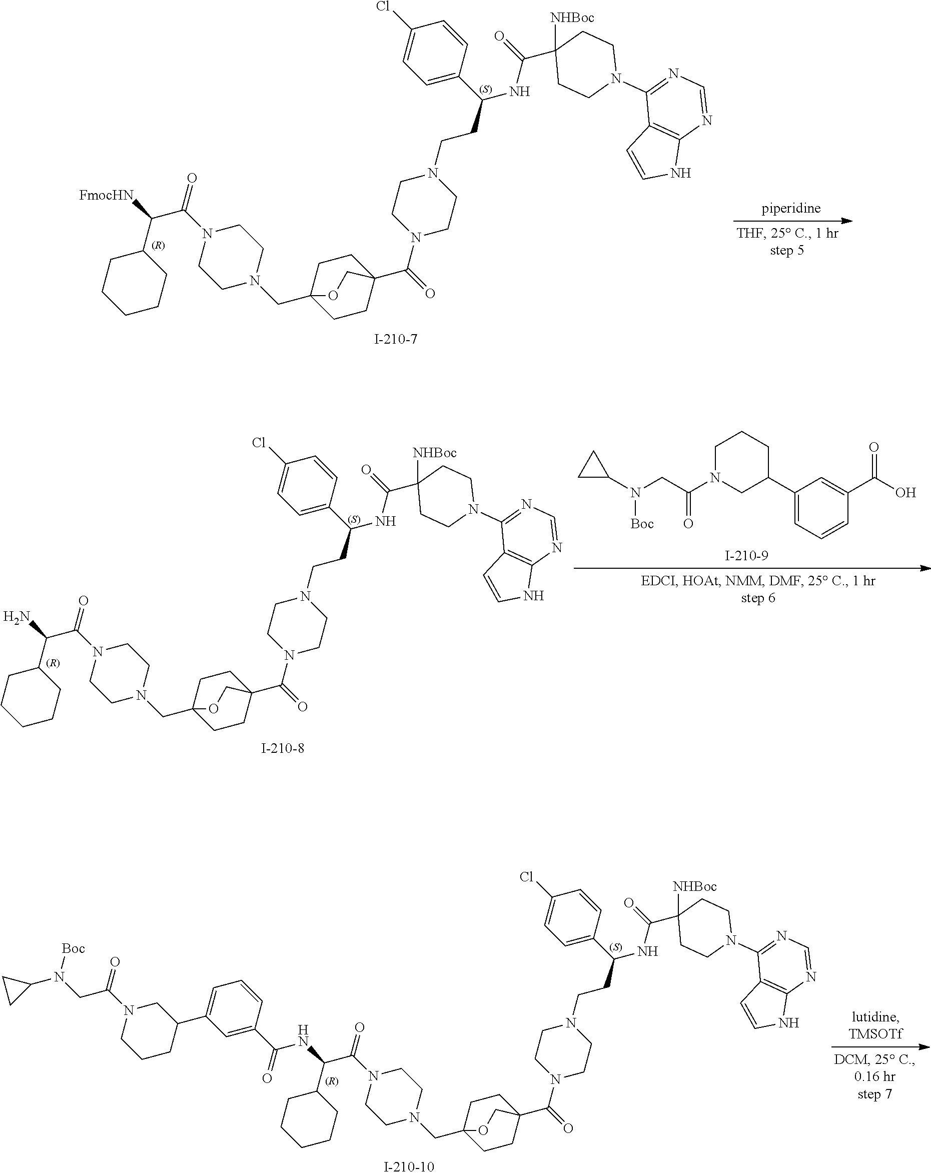

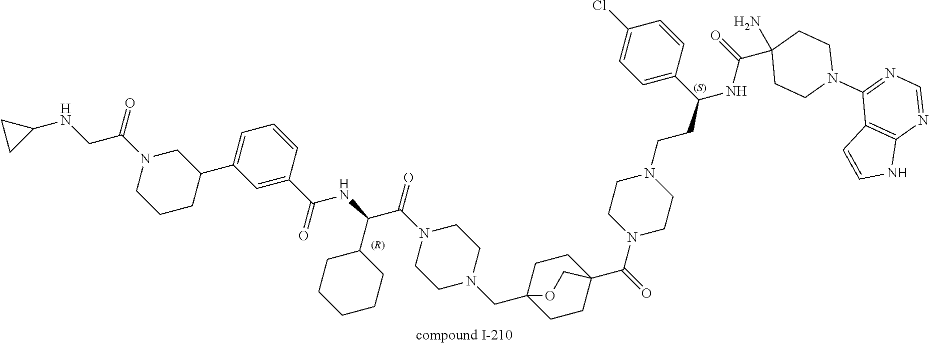

### Step 1: Synthesis of I-210-3

To a solution of I-210-1 (1.5 g, 4.92 mmol, 1 eq, HCl) and I-210-2 (1.87 g, 4.92 mmol, 1.87 mL, 1 eq) in DMF (15 mL) was added EDCI (2.83 g, 14.76 mmol, 3 eq), HOAt (669.81 mg, 4.92 mmol, 688.40 μL, 1 eq) and NMM (2.49 g, 24.61 mmol, 2.71 mL, 5 eq). The mixture was stirred at 25° C. for 1 hr. The mixture was washed with water (30 mL) and extract with EA (20 mL*3). The combined organic layers dried over sodium sulfate, filtered, and concentrated under reduced pressure to give a residue. The residue was purified by flash silica gel chromatography (ISCO®; 80 g SepaFlash® Silica Flash Column, Eluent of 0˜50% EA/PE @ 100 mL/min) and then the eluent was concentrated in vacuo to give product. I-210-3 (3.2 g, 4.73 mmol, 96.02% yield, 93% purity) was obtained as yellow oil. LCMS (Method D): R^{t}=0.426 min, [M+H]⁺=630.4. SFC: R^{t}=1.473 min. ¹H NMR (400 MHZ, METHANOL-d4) δ=7.79 (d, J=7.6 Hz, 2H), 7.69-7.62 (m, 2H), 7.39-7.36 (m, 2H), 7.34-7.27 (m, 2H), 4.94-4.86 (m, 1H), 4.38-4.35 (m, 2H), 4.26-4.18 (m, 1H), 3.89 (s, 2H), 3.67 (s, 1H), 3.64 (s, 3H), 3.61-3.45 (m, 2H), 3.39 (s, 1H), 2.88-2.56 (m, 1H), 2.55 (s, 1H), 2.52-2.49 (m, 1H), 2.36-2.23 (m, 3H), 1.99-1.89 (m, 6H), 1.79-1.55 (m, 8H), 1.28 (d, J=4.0 Hz, 1H), 1.24-1.21 (m, 1H), 1.20-1.11 (m, 1H), 1.06-0.98 (m, 2H).

### Step 2: Synthesis of I-210-4

To a solution of I-210-3 (1.4 g, 2.22 mmol, 1 eq) in THF (7 mL) and H₂O (7 mL) was added LiOH·H₂O (279.83 mg, 6.67 mmol, 3 eq). The mixture was stirred at 25° C. for 12 hr. A solution of I-210-4 (1.4 g, crude) in THF (7 mL) and H₂O (7 mL) was obtained as a yellow liquid, and it used into next step directly. LCMS (Method D): R^{t}=0.145 min, [M+H]⁺=394.3.

### Step 3: Synthesis of I-210-5

A mixture of I-210-4 (1.4 g, 3.56 mmol, 1 eq) in THF (7 mL) and H₂O (7 mL) was added FMOC-OSU (2.40 g, 7.12 mmol, 2 eq) at 25° C. The result mixture was stirred at 25° C. for 1 hr. The mixture was acidified to pH=5˜6 with 1N HCl, and the mixture was diluted with water (5 mL) and extract with EA (8 mL*3). The combined organic layers dried over sodium sulfate, filtered, and concentrated under reduced pressure to give a residue. The residue was purified by flash silica gel chromatography (ISCO®; 40 g SepaFlash® Silica Flash Column, Eluent of 0˜100% EA/MeOH (60 mL/min) and then the eluent was concentrated in vacuo to give product. I-210-5 (1.5 g, 2.24 mmol, 63.00% yield, 92% purity) was obtained as a white solid. LCMS (Method D): R^{t}=0.397 min, [M+H]⁺=616.3.

### Step 4: Synthesis of I-210-7

To a solution of I-210-6 (1 g, 1.62 mmol, 1 eq) and I-210-5 (969.78 mg, 1.62 mmol, 1 eq) in DMF (10 mL) was added EDCI (933.98 mg, 4.87 mmol, 3 eq), HOAt (221.05 mg, 1.62 mmol, 227.18 μL, 1 eq) and NMM (821.32 mg, 8.12 mmol, 892.74 μL, 5 eq). The mixture was stirred at 25° C. for 1 hr. The mixture was diluted with water (10 mL) and extract with EA (10 mL*3). The combined organic layers dried over sodium sulfate, filtered, and concentrated under reduced pressure to give a residue. The residue was purified by flash silica gel chromatography (ISCO®; 80 g SepaFlash® Silica Flash Column, Eluent of 0˜25% MeOH/DCM @ 100 mL/min) and then the eluent was concentrated in vacuo to give product. I-210-7 (1.2 g, 933.98 μmol, 57.51% yield, 93% purity) was obtained as a white solid. LCMS (Method D): R^{t}=0.398 min, [M/2+H]⁺=598.3. SFC: R^{t}=2.352 min.

### Step 5: Synthesis of I-210-8

To a solution of I-210-7 (1.2 g, 1.00 mmol, 1 eq) in THF (12 mL) was added piperidine (1.29 g, 15.19 mmol, 1.5 mL, 15.12 eq). The mixture was stirred at 25° C. for 1 hr. The reaction mixture was concentrated in vacuo. The residue was purified by flash silica gel chromatography (ISCO®; 40 g SepaFlash® Silica Flash Column, Eluent of 0˜50% MeOH (NH₃·H₂O)/EA (a, 60 mL/min) and then the eluent was concentrated in vacuo to give product. I-210-8 (680 mg, 608.23 μmol, 60.56% yield, 87% purity) was obtained as a yellow solid. LCMS (Method D): R^{t}=0.279 min, [M/2+H]=487.2.

### Step 6: Synthesis of I-210-10

To a solution of I-210-8 (650 mg, 668.28 μmol, 1 eq) and I-210-9 (268.97 mg, 668.28 μmol, 1 eq) in DMF (6.5 mL) was added EDCI (384.33 mg, 2.00 mmol, 3 eq), HOAt (90.96 mg, 668.28 μmol, 93.48 μL, 1 eq) and NMM (337.97 mg, 3.34 mmol, 367.36 μL, 5 eq). The mixture was stirred at 25° C. for 1 hr. The mixture was diluted with water (8 mL) and extracted with EA (10 mL*3). The combined organic layers dried over sodium sulfate, filtered, and concentrated under reduced pressure to give a residue. The residue was purified by flash silica gel chromatography (ISCO®; 80 g SepaFlash R: Silica Flash Column, Eluent of 0˜100% MeOH/EA (a, 100 mL/min) and then the eluent was concentrated in vacuo to give product. I-210-10 (820 mg, 598.18 μmol, 89.51% yield, 99% purity) was obtained as a yellow solid. LCMS (Method D): R^{t}=0.394 min, [M/2+H]=1357.5. SFC: R^{t}=2.272 min, 4.076 min.

### Step 7: Synthesis of I-210

To a solution of I-210-10 (400 mg, 294.74 μmol, 1 eq) in DCM (4 mL) was added lutidine (9.47 mg, 88.42 μmol, 10.30 μL, 0.3 eq) and TMSOTf (98.26 mg, 442.11 μmol, 79.89 UL, 1.5 eq). The mixture was stirred at 0° C. for 10 min. The mixture was basified to pH=7˜8 with NH₃·H₂O and the mixture was concentrated in vacuo. The residue was purified by reversed phase column (0.1% NH₃·H₂O condition), the eluent was concentrated under reduced pressure to remove ACN and lyophilized to give product. I-210 (156.4 mg, 134.17 μmol, 45.52% yield, 99.249% purity) was obtained as a white solid. LCMS (Method D): R^{t}=0.278 min, [M+H]⁺=1156.3. SFC: R^{t}=5.718 min, 7.153 min. ¹H NMR (400 MHZ, METHANOL-d4) δ=8.12 (s, 1H), 7.76 (s, 1H), 7.73-7.68 (m, 1H), 7.51-7.46 (m, 1H), 7.45-7.40 (m, 1H), 7.32 (s, 4H), 7.12 (d, J=3.6 Hz, 1H), 6.62 (d, J=3.6 Hz, 1H), 4.98-4.96 (m, 1H), 4.90 (d, J=8.8 Hz, 1H), 4.58 (d, J=9.6 Hz, 1H), 4.53-4.46 (m, 2H), 3.94 (s, 2H), 3.74-3.63 (m, 8H), 3.57 (d, J=2.4 Hz, 2H), 3.53-3.47 (m, 1H), 3.22-3.13 (m, 1H), 2.87-2.78 (m, 1H), 2.76-2.69 (m, 1H), 2.61 (s, 2H), 2.55-2.50 (m, 2H), 2.48-2.43 (m, 2H), 2.42-2.37 (m, 2H), 2.35-2.31 (m, 2H), 2.29 (s, 2H), 2.22-2.14 (m, 4H), 2.08-2.03 (m, 2H), 1.98-1.93 (m, 3H), 1.87 (d, J=5.2 Hz, 7H), 1.78 (d, J=12.0 Hz, 2H), 1.66-1.65 (m, 5H), 1.62-1.52 (m, 3H), 1.35-1.19 (m, 4H), 1.10-1.05 (m, 2H), 0.49-0.44 (m, 2H), 0.42-0.37 (m, 2H).

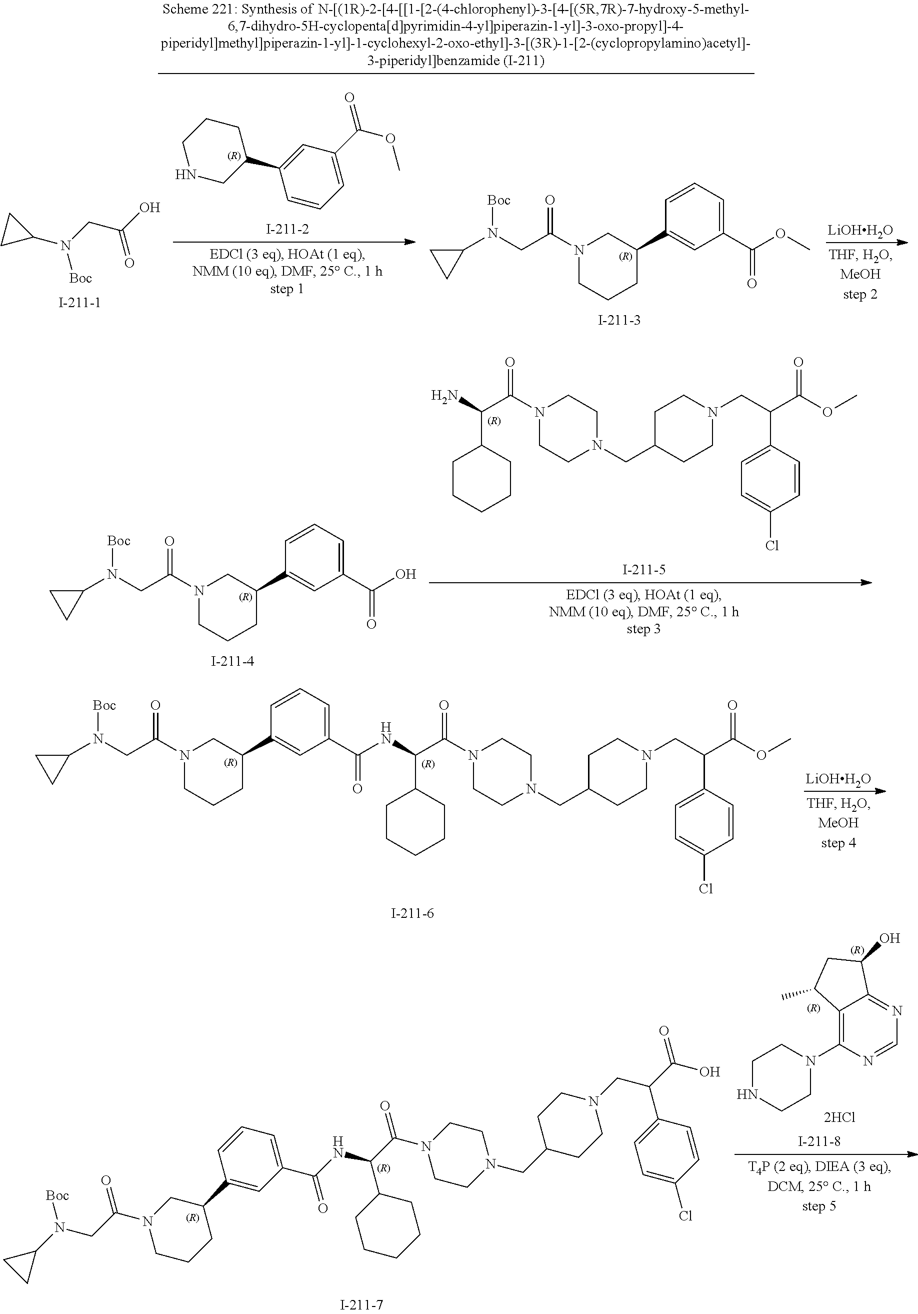

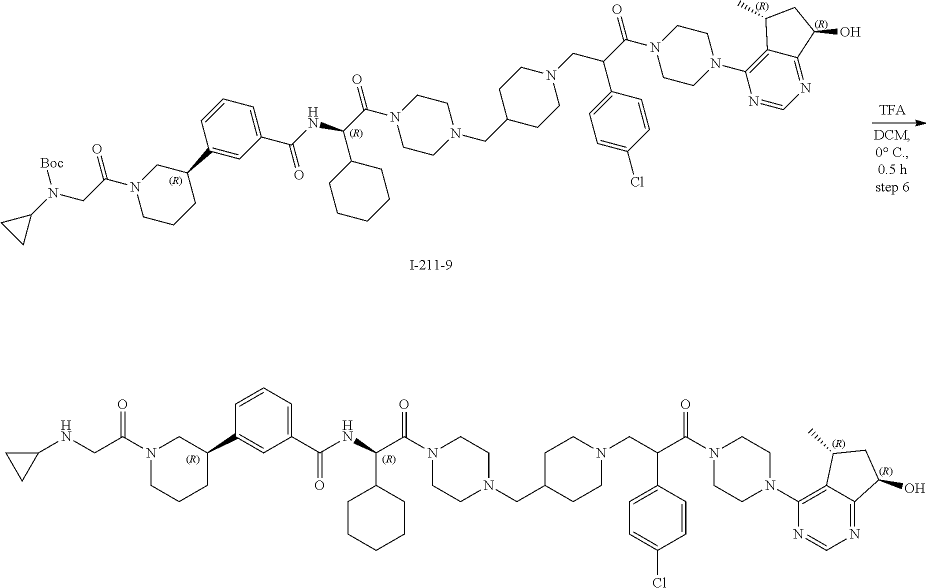

### Step 1: Synthesis of I-211-3

To a solution of I-211-2 (522.78 mg, 2.04 mmol, 1.1 eq, HCl) and I-211-1 (400 mg, 1.86 mmol, 1 eq) in DMF (5 mL) was added EDCI (1.07 g, 5.58 mmol, 3 eq), HOAt (252.94 mg, 1.86 mmol, 259.96 μL, 1 eq) and NMM (1.88 g, 18.58 mmol, 2.04 mL, 10 eq). The mixture was stirred at 25° C. for 1 hr. The reaction mixture was quenched by addition H₂O (10 mL), and then diluted with EA (10 mL) and extracted with EA (10 mL*3). The combined organic layers were washed with brine (10 mL*3), dried over anhydrous Na₂SO₄, filtered, and concentrated under reduced pressure to give a residue. The residue was purified by flash silica gel chromatography (ISCO®; 20 g SepaFlash® Silica Flash Column, Eluent of 0˜100% Ethyl acetate/Petroleum ether gradient (40 mL/min). The eluent was concentrated under reduced pressure to give a product. I-211-3 (650 mg, 1.54 mmol, 83.14% yield, 99% purity) was obtained as a white solid. LCMS: R^{t}=0.562 min, [M+H-Boc]⁺=317.2.

### Step 2: Synthesis of I-211-4

To a solution of I-211-3 (350 mg, 840.32 μmol, 1 eq) in THF (3.5 mL), H₂O (3.5 mL), MeOH (3.5 mL) was added LiOH·H₂O (105.79 mg, 2.52 mmol, 3 eq). The mixture was stirred at 25° C. for 2 hr. The reaction mixture was adjusted to a pH=6 with HCl (1M) and then extracted with EA 15 mL (15 mL*3). The combined organic layers were dried over anhydrous Na₂SO₄, filtered, and concentrated under reduced pressure to give a residue. The crude product was used into the next step without further purification. I-211-4 (330 mg, crude) was obtained as a white solid. LCMS: R^{t}=0.350 min, [M+H-Boc]⁺=303.2.

### Step 3: Synthesis of I-211-6

To a solution of I-211-4 (330 mg, 819.91 μmol, 1 eq), I-211-5 (425.63 mg, 819.91 μmol, 1 eq) in DMF (3 mL) was added EDCI (471.53 mg, 2.46 mmol, 3 eq) and HOAt (111.60 mg, 819.91 μmol, 114.70 μL, 1 eq), NMM (829.32 mg, 8.20 mmol, 901.43 μL, 10 eq). The mixture was stirred at 25° C. for 1 hr. The reaction mixture was quenched by addition water (5 mL) at 25° C., and extracted with EA (5 mL*3). The combined organic layers were washed with sat. NaCl (5 mL*3), dried over Na₂SO₄, filtered, and concentrated under reduced pressure to give a residue. The crude product was used into the next step without further purification. I-211-6 (520 mg, crude) was obtained as a white solid. LCMS: R^{t}=0.873 min, [M+H]⁺=905.6.

### Step 4: Synthesis of I-211-7

To a solution of I-211-6 (500 mg, 553.35 μmol, 1 eq) in H₂O (5 mL), THF (5 mL), MeOH (5 mL) was added LiOH·H₂O (23.22 mg, 553.35 μmol, 1 eq). The mixture was stirred at 25° C. for 1 hr. The reaction mixture was adjusted to a pH=6 with aq. HCl (1M) and then extracted with EA (15 mL*3). The combined organic layers were dried over anhydrous Na₂SO₄, filtered, and concentrated under reduced pressure to give a residue. The residue was purified by reversed phase (26.8*125 mm, 80 g of XB—C18, 20-40 μm, 120 Å) Mobile phase: A for H₂O (0.1% FA v/v) and B for acetonitrile; Gradient: B 30%-50% in 30 min: Flow rate: 60 ml/min; Column temperature: R.T. Wavelength: 220 nm/254 nm). The eluent was concentrated to remove organic solvents and then lyophilization. I-211-7 (380 mg, 401.55 μmol, 72.57% yield, 94% purity) was obtained as a white solid. LCMS: R^{t}=0.579 min, [M+H]⁺=889.6. SFC: R^{t}=0.716 min, 0.992 min.

### Step 5: Synthesis of I-211-9

To a solution of I-211-7 (370 mg, 415.94 μmol, 1 eq), I-211-8 (127.78 mg, 415.94 μmol, 1 eq, 2HCl) in DCM (2.5 mL) was added T₄P (299.69 mg, 831.87 μmol, 2 eq) and DIEA (161.27 mg, 1.25 mmol, 217.34 μL, 3 eq). The mixture was stirred at 25° C. for 1 hr. The reaction mixture was quenched by addition water (5 mL) at 25° C., and extracted with EA (5 mL*3). The combined organic layers were washed with brine (5 mL*3), dried over Na₂SO₄, filtered, and concentrated under reduced pressure to give a residue. The product was purified by prep-HPLC (column: Waters Xbridge Prep OBD C18 150*40 mm*10 um; mobile phase: [water (NH4HCO3)-ACN]; gradient: 53%-83% B over 15 min) and the eluent was concentrated to remove MeCN and then lyophilization. I-211-9 (70 mg, 63.30 μmol, 15.22% yield, 100% purity) was obtained as a white solid. LCMS: R^{t}=0.802 min, [M+H]⁺=1105.8. SFC: R^{t}=0.848 min, 1.247 min.

### Step 6: Synthesis of I-211

To a solution of I-211-9 (30 mg, 27.13 μmol, 1 eq) in DCM (0.5 mL) was added TFA (307.00 mg, 2.69 mmol, 0.2 mL, 99.25 eq). The mixture was stirred at 0° C. for 0.5 hr. The reaction mixture was concentrated under reduced pressure to give a residue. The residue was purified by prep-HPLC (column: Waters Xbridge Prep OBD C18 150*40 mm*10 um; mobile phase: [water (NH₄HCO₃)-ACN]; gradient: 62%-92% B over 15 min) and the eluent was concentrated to remove MeCN and then lyophilization. I-211 (23 mg, 20.54 μmol, 75.71% yield, 100% purity, TFA) was obtained as a white solid. LCMS: R^{t}=0.733 min, [M+H]⁺=1005.7. SFC: R^{t}=2.329 min, 3.497 min. ¹H NMR (400 MHZ, METHANOL-d4) δ=8.57 (d, J=2.8 Hz, 1H), 7.83-7.69 (m, 2H), 7.57-7.42 (m, 4H), 7.37 (m, 2H), 5.28 (m, 1H), 4.75-4.67 (m, 1H), 4.63-4.51 (m, 1H), 4.34-4.07 (m, 5H), 4.02-3.86 (m, 4H), 3.85-3.59 (m, 7H), 3.55-3.33 (m, 5H), 3.28-3.04 (m, 7H), 3.04-2.67 (m, 4H), 2.34-2.15 (m, 3H), 2.08 (m, 3H), 2.01-1.86 (m, 4H), 1.83-1.63 (m, 7H), 1.39-1.06 (m, 9H), 0.96-0.87 (m, 4H). ¹⁹F NMR (377 MHz, METHANOL-d4) δ=−76.94.

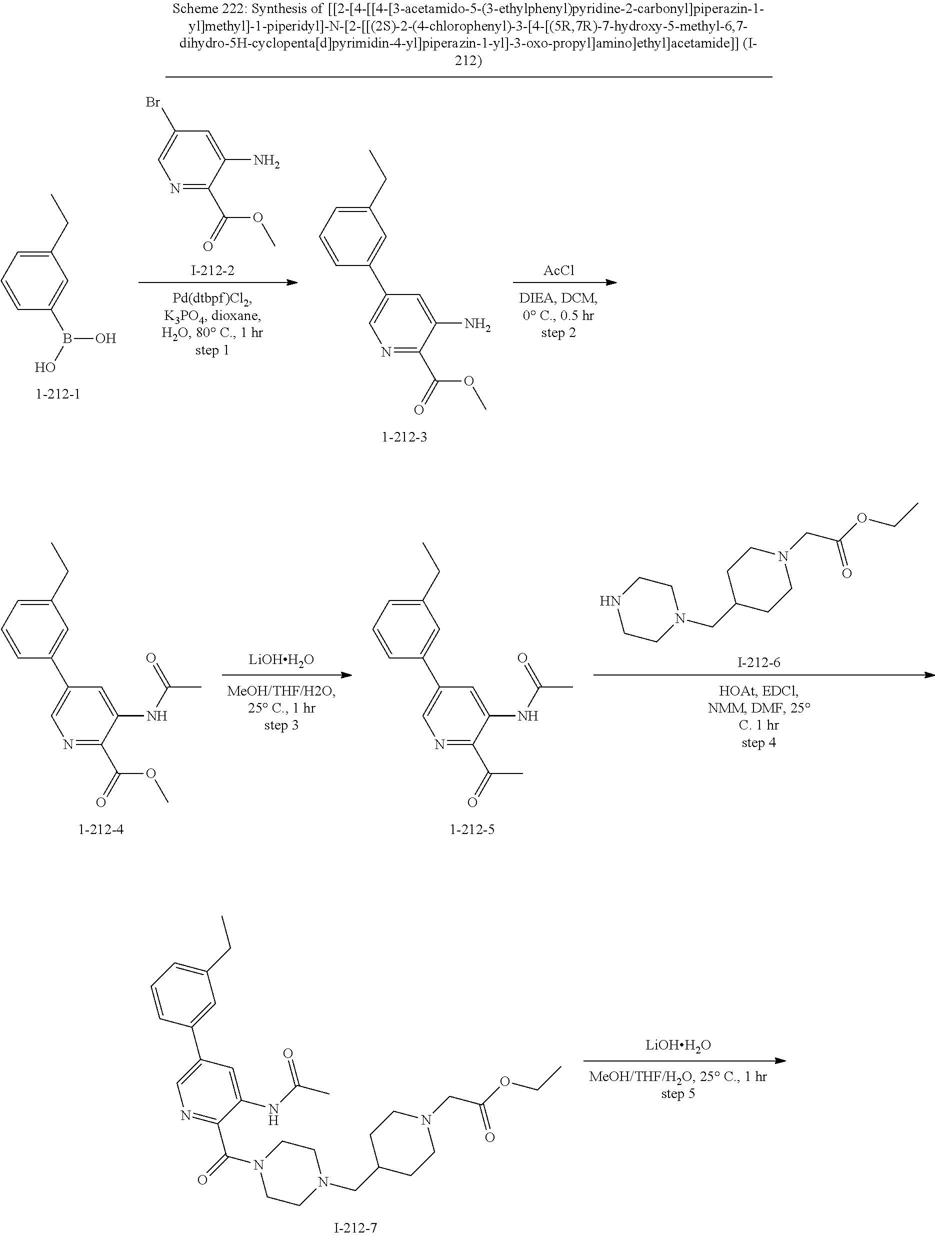

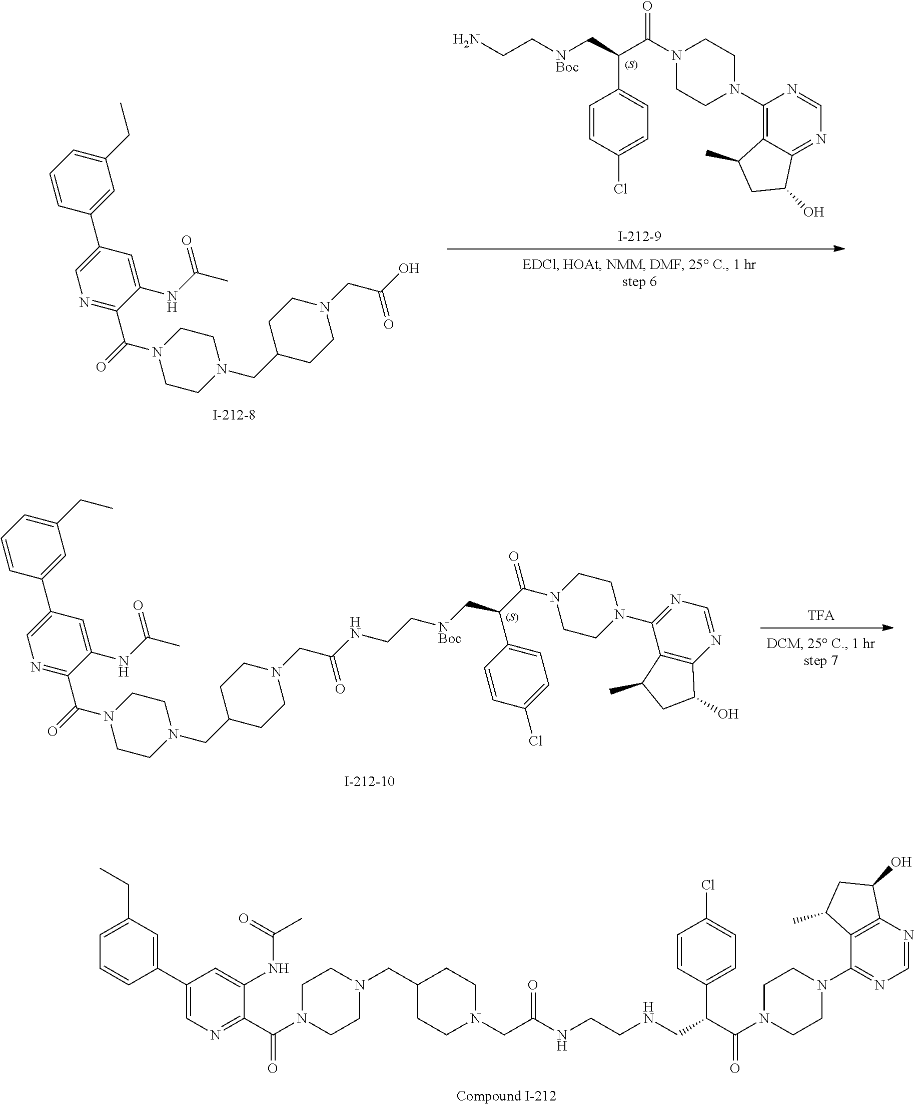

### Step 1: Synthesis of I-212-3

To a mixture of I-212-2 (10 g, 43.28 mmol, 1 eq) and I-212-1 (9.74 g, 64.92 mmol, 1.5 eq) in dioxane (100 mL) and H₂O (20 mL) was added K₃PO₄ (27.56 g, 129.84 mmol, 3 eq) and Pd(dtbpf)Cl₂ (2.82 g, 4.33 mmol, 0.1 eq). Then the mixture was stirred at 80° C. for 1 hour under N₂. The reaction mixture was diluted with water 200 mL and extracted with EA 120 mL (60 mL*2). The combined organic layers were concentrated under reduced pressure to give a residue. The residue was purified by flash silica gel chromatography ((ISCO®; 120 g SepaFlash® Silica Flash Column, Eluent of 0˜10% Dichloromethane: Methanol @ 60 mL/min)). Then The eluent was concentrated under reduced pressure to give the I-212-3 (12 g, 41.37 mmol, 95.58% yield, 88.351% purity) as a brown solid. LCMS (Method D): R^{t}=0.344 min, [M+H]⁺=257.0.

### Step 2: Synthesis of I-212-4

To a solution of I-212-3 (900 mg, 3.51 mmol, 1 eq) in DCM (9 mL) was added DIEA (1.36 g, 10.53 mmol, 1.83 mL, 3 eq). The solution was cooled to 0° C., and acetyl chloride (303.21 mg, 3.86 mmol, 274.65 μL, 1.1 eq) was added. The mixture was stirred at 0° C. for 0.5 hr. The reaction mixture was concentrated by reducing pressure to obtain the crude product. The crude product was used the next step without further purification. I-212-4 (1 g, crude) was obtained as a brown solid. LCMS (Method D): R^{t}=0.423 min, [M+H]⁺=299.2.

### Step 3: Synthesis of I-212-5

To a solution of I-212-4 (1 g, 3.35 mmol, 1 eq) in MeOH (3 mL), THF (3 mL) and H₂O (3 mL) was added LiOH·H₂O (421.98 mg, 10.06 mmol, 3 eq). The mixture was stirred at 25° C. for 2 hr. The mixture was adjusted to pH=5 with HCl (1 M) and then extracted with EA 15 mL (5 mL*3), dried over Na₂SO₄, filtered, and concentrated under reduced pressure to give a residue. The crude product was purified by flash silica gel chromatography (ISCO®; 20 g SepaFlash® Silica Flash Column, Eluent of 0˜100% Petroleum ether/Ethyl acetate @; 40 mL/min). The eluent was concentrated under reduced pressure to give I-212-5 (300 mg, 1.06 mmol, 31.48% yield) as a brown solid. LCMS (Method D): R^{t}=0.355 min, [M+H]⁺=285.1.

### Step 4: Synthesis of I-212-7

To a solution of I-212-5 (150 mg, 527.59 μmol, 1 eq) in DMF (1.5 mL) was added HOBt (71.29 mg, 527.59 μmol, 1 eq), EDCI (303.42 mg, 1.58 mmol, 3 eq), NMM (266.82 mg, 2.64 mmol, 290.03 μL, 5 eq) and I-212-6 (142.12 mg, 464.70 μmol, 8.81e-1 eq, HCl). The mixture was stirred at 25° C. for 1 hr. The reaction mixture was purified by reversed phase column (Neutral condition), which was concentrated and lyophilized to afford I-212-7 (150 mg, 280.02 μmol, 53.07% yield) as a yellow oil. LCMS (Method D): R^{t}=0.645 min, [M+H]⁺=536.3.

### Step 5: Synthesis of I-212-8

To a solution of I-212-7 (150 mg, 280.02 μmol, 1 eq) in MeOH (0.5 mL), THF (0.5 mL) and H₂O (0.5 mL) was added LiOH·H₂O (35.25 mg, 840.06 μmol, 3 eq). The mixture was stirred at 25° C. for 1 hr. The reaction mixture was concentrated under vacuum to remove MeOH and THF. Then the residue was diluted with H₂O (10 mL) and then lyophilized to afford the crude product. The crude product was used the next step without further purification. I-212-8 (120 mg, 236.40 μmol, 84.42% yield) was obtained as a white solid. LCMS (Method D): R^{t}=0.263 min, [M+H]⁺=508.3.

### Step 6: Synthesis of I-212-10

To a solution of I-212-8 (20 mg, 39.40 μmol, 1 eq) in DMF (0.2 mL) was added HOAt (5.36 mg, 39.40 μmol, 5.51 μL, 1 eq), EDCI (22.66 mg, 118.20 μmol, 3 eq), NMM (19.93 mg, 197.00 μmol, 21.66 μL, 5 eq) and I-212-9 (22.03 mg, 39.40 μmol, 1 eq). The mixture was stirred at 25° C. for 1 hr. The reaction liquid was concentrated by reducing pressure to obtain the crude product. The crude product was purified by reverse-phase column (0.1% FA condition), which was concentrated and lyophilized to afford I-212-10 (70 mg, crude, FA) as a yellow solid. LCMS (Method D): R^{t}=0.379 min, [M+H]⁺=1048.5. SFC: R^{t}=2.160 min.

### Step 7: Synthesis of I-212

To a solution of I-212-10 (60 mg, 54.81 μmol, 1 eq, FA) in DCM (0.4 mL) was added TFA (307.00 mg, 2.69 mmol, 0.2 mL, 49.12 eq). The mixture was stirred at 25° C. for 1 hr. The reaction liquid was concentrated by reducing pressure to obtain the crude product. The crude product was purified by reversed phase column (0.1% TFA condition) which was concentrated and lyophilized to afford I-212 (19.18 mg, 18.00 μmol, 32.85% yield, 99.747% purity, TFA) as a yellow solid. LCMS (Method D): R^{t}=0.280 min, [M+H]⁺=948.4. SFC: R^{t}=0.604 min. ¹H NMR (400 MHZ, METHANOL-d4) δ=8.66 (s, 1H), 8.57 (s, 1H), 8.02 (s, 1H), 7.55-7.49 (m, 2H), 7.48-7.41 (m, 3H), 7.39-7.31 (m, 3H), 5.29 (t, J=7.6 Hz, 1H), 4.64-4.56 (m, 1H), 4.22-4.14 (m, 1H), 4.00 (s, 3H), 3.91 (s, 3H), 3.83 (s, 3H), 3.70-3.64 (m, 5H), 3.62 (d, J=5.6 Hz, 3H), 3.46 (s, 5H), 3.26 (t, J=5.2 Hz, 3H), 3.21 (d, J=6.4 Hz, 4H), 2.78-2.72 (m, 2H), 2.35-2.24 (m, 2H), 2.23-2.08 (m, 6H), 1.83-1.67 (m, 2H), 1.52 (s, 1H), 1.29 (t, J=7.6 Hz, 3H), 1.22-1.14 (m, 3H). ¹⁹F NMR (377 MHz, METHANOL-d4), δ=−76.918 (TFA).

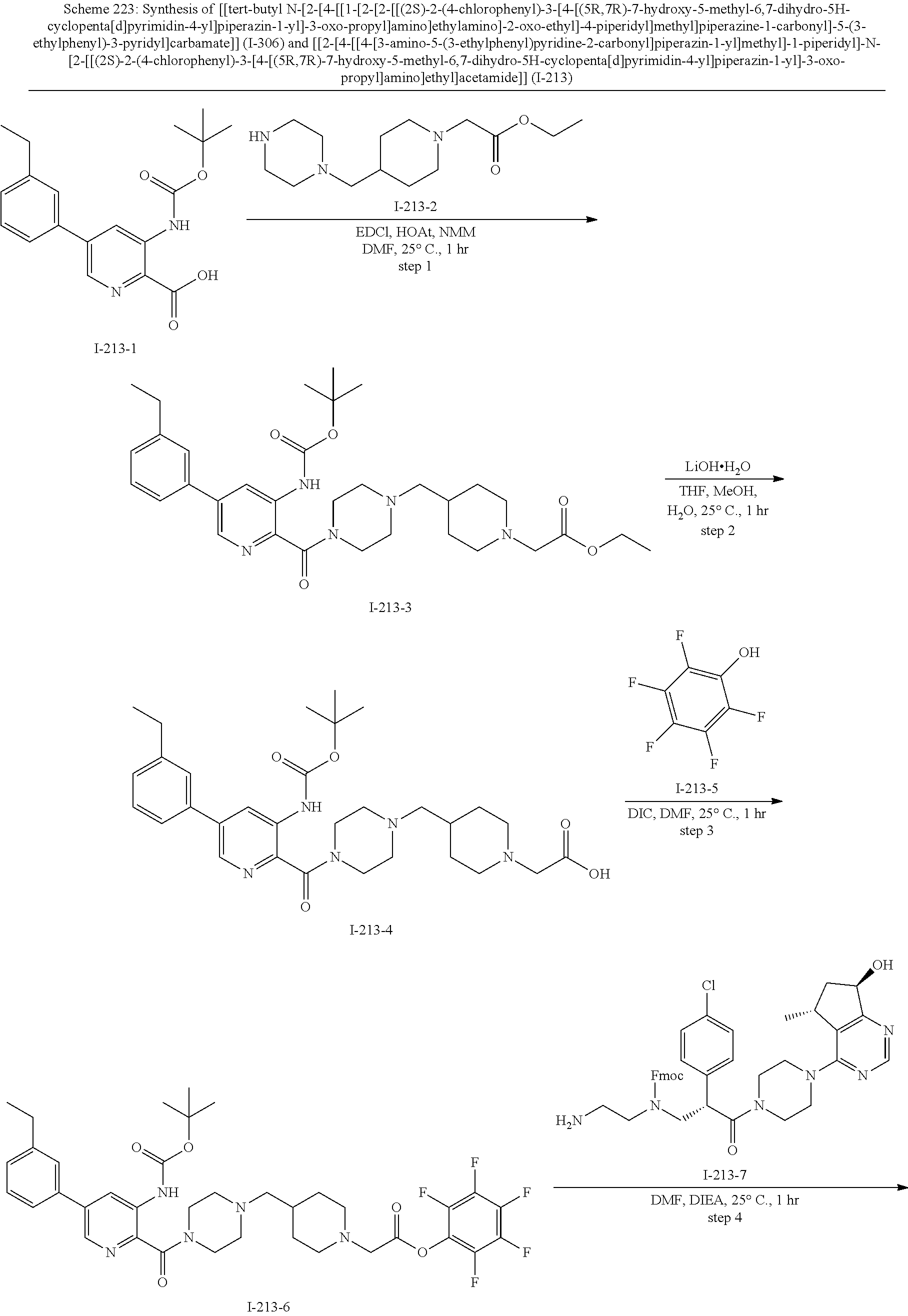

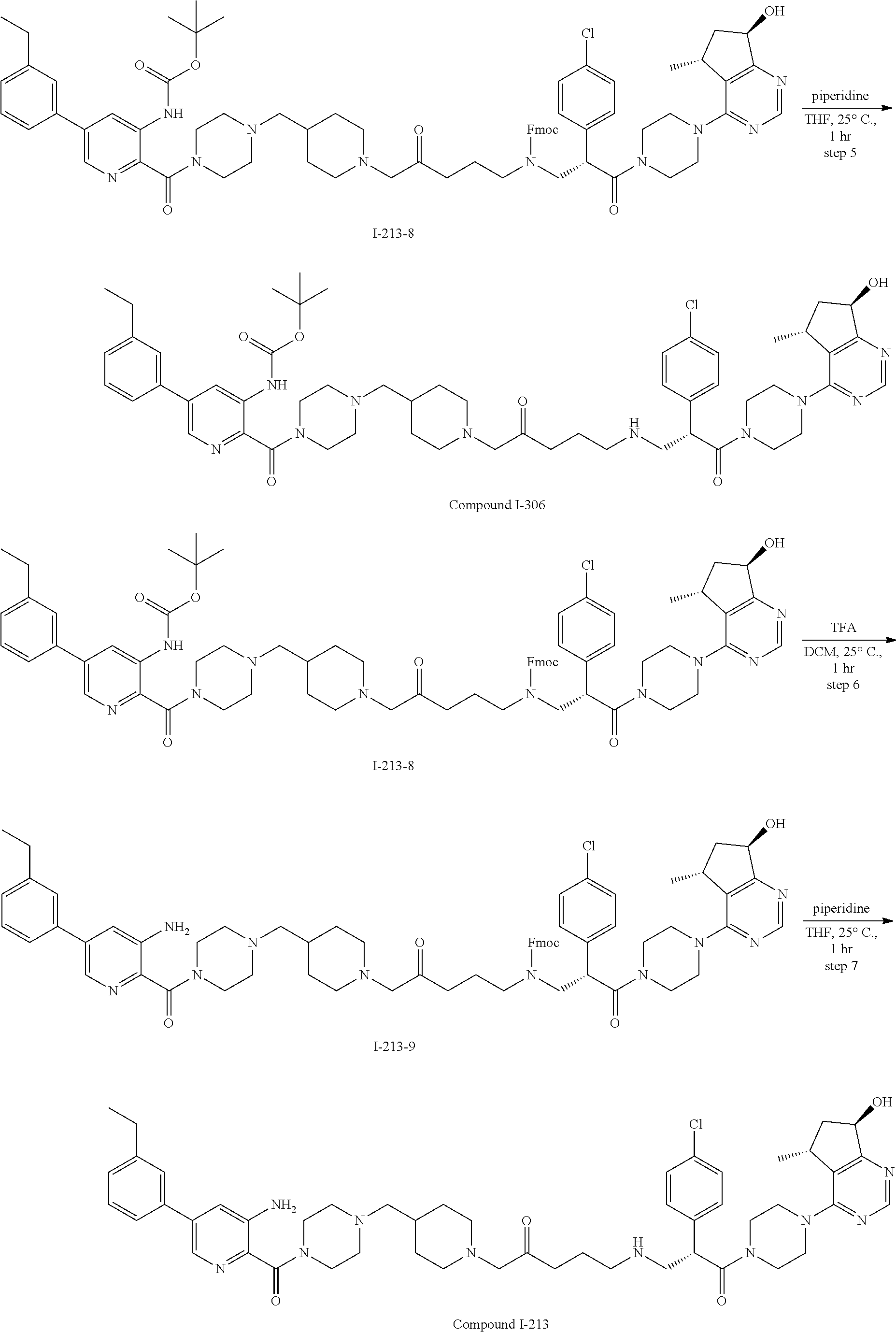

### Step 1: Synthesis of I-213-3

To a solution of I-213-1 (200 mg, 584.13 μmol, 1 eq) in DMF (2 mL) was added HOAt (79.51 mg, 584.13 μmol, 81.71μ, 1 eq), EDCI (335.94 mg, 1.75 mmol, 3 eq), NMM (295.42 mg, 2.92 mmol, 321.10 μL, 5 eq) and I-213-2 (178.65 mg, 584.13 μmol, 1 eq, HCl). The mixture was stirred at 25° C. for 1 hr. The residue was diluted with H₂O 5 mL and extracted with EA 15 mL (5 mL*3), dried over Na₂SO₄, filtered, and concentrated under reduced pressure to give a residue. The combined crude product was purified by flash silica gel chromatography (ISCO®; 20 g SepaFlash® Silica Flash Column, Eluent of 0˜100% Dichloromethane/Methanol (@; 60 mL/min). The eluent was concentrated under reduced pressure to afford I-213-3 (150 mg, 252.63 μmol, 43.25% yield) as a brown oil. LCMS (Method D): R^{t}=0.355 min, [M+H]⁺=599.4.

### Step 2: Synthesis of I-213-4

To a solution of I-213-3 (150 mg, 252.63 μmol, 1 eq) in MeOH (0.5 mL), THF (0.5 mL) and H: 0 (0.5 mL) was added LiOH·H₂O (31.80 mg, 757.89 μmol, 3 eq). The mixture was stirred at 25° C. for 1 hr. The reaction mixture was concentrated by reducing pressure to obtain the crude product. The crude product was purified by reversed phase column (Neutral condition), which was concentrated and lyophilized to afford I-213-4 (80 mg, 136.58 μmol, 54.06% yield, 96.577% purity) as a white solid. LCMS (Method D): R^{t}=0.366 min, [M+H]⁺=566.3.

### Step 3: Synthesis of I-213-6

To a solution of I-213-4 (50 mg, 88.39 μmol, 1 eq) in DMF (0.5 mL) was added I-213-5 (24.40 mg, 132.58 μmol, 1.5 eq) and DIC (16.73 mg, 132.58 μmol, 20.53 μL, 1.5 eq). The mixture was stirred at 25° C. for 1 hr. The residue was diluted with H₂O 5 mL and extracted with EA 15 mL (5 mL*3), dried over Na₂SO₄, filtered, and concentrated under reduced pressure to give a residue. The crude product was used the next step without further purification. I-213-6 (60 mg, crude) was obtained as a yellow oil. LCMS (Method D): R^{t}=0.389 min, [M+H]⁺=732.6.

### Step 4: Synthesis of I-213-8

To a solution of I-213-6 (60 mg, 82.00 μmol, 1 eq) in DMF (0.6 mL) was added DIEA (21.19 mg, 163.99 μmol, 28.56 μL, 2 eq) and I-213-7 (58.85 mg, 82.00 μmol, 1 eq, HCl). The mixture was stirred at 25° C. for 1 hr. The reaction liquid was concentrated by reducing pressure to obtain the crude product. The crude product was purified by reversed phase column (0.1% FA condition), which was concentrated and lyophilized to afford I-213-8 (60 mg, 47.06 μmol, 57.40% yield, FA) as a white solid. LCMS (Method D):

R^{t}=0.389 min, [M+H]⁺=1228.5.

### Step 5: Synthesis of I-306

To a solution of I-213-8 (20 mg, 15.69 μmol, 1 eq, FA) in THF (0.2 mL) was added piperidine (33.24 mg, 390.42 μmol, 38.56 μL, 24.89 eq). The mixture was stirred at 25° C. for 1 hr. The reaction liquid was concentrated by reducing pressure to obtain the crude product. The crude product was purified by reversed phase column (Neutral condition), which was concentrated and lyophilized to afford I-306 (7.89 mg, 7.84 μmol, 49.96% yield, 100.00% purity) as a white solid. LCMS (Method D): R^{t}=0.331 min, [M+H]⁺=1006.7. SFC: R^{t}=1.071 min. ¹H NMR (400 MHz, METHANOL-d4) δ=8.53 (s, 1H), 8.41 (s, 2H), 7.54-7.46 (m, 2H), 7.42 (t, J=7.6 Hz, 1H), 7.40-7.34 (m, 2H), 7.34-7.27 (m, 3H), 4.96 (t, J=6.8 Hz, 1H), 4.20-4.17 (m, 1H), 3.93-3.85 (m, 1H), 3.83-3.66 (m, 4H), 3.64-3.40 (m, 7H), 3.37-3.32 (m, 2H), 3.28-3.08 (m, 4H), 2.97 (s, 2H), 2.85 (d, J=5.6 Hz, 2H), 2.80-2.69 (m, 5H), 2.57 (s, 2H), 2.49 (s, 2H), 2.27 (d, J=6.8 Hz, 2H), 2.18-2.09 (m, 3H), 2.08-2.00 (m, 1H), 1.83-1.72 (m, 2H), 1.54 (s, 9H), 1.29 (t, J=7.6 Hz, 5H), 1.17-1.07 (m, 3H).

### Step 6: Synthesis of I-213-9

To a solution of I-213-8.1 (40 mg, 31.37 μmol, 1 eq, FA) in DCM (0.3 mL) was added TFA (153.50 mg, 1.35 mmol, 0.1 mL, 42.91 eq). The mixture was stirred at 25° C. for 1 hr. The reaction mixture was concentrated by reducing pressure to obtain the crude product. The crude product was used the next step without further purification. I-213-9 (36 mg, crude, TFA) was obtained as a yellow oil. LCMS (Method D): R^{t}=0.373 min, [M+H]⁺=1129.1.

### Step 7: Synthesis of I-213

To a solution of I-213-9 (36 mg, 28.97 μmol, 1 eq, TFA) in THF (0.4 mL) was added piperidine (68.98 mg, 810.08 μmol, 0.08 mL, 27.97 eq). The mixture was stirred at 25° C. for 1 hr. The reaction mixture was concentrated by reducing pressure to obtain the crude product. The crude product was purified by reversed phase column (Neutral condition), which was concentrated and lyophilized to afford I-213 (15.77 mg, 16.69 μmol, 57.63% yield, 95.964% purity) as a white solid. LCMS: R^{t}=0.296 min, [M+H]⁺=906.4. SFC: R^{t}=1.727 min. ¹H NMR (400 MHZ, METHANOL-d4) δ=8.41 (s, 1H), 8.08 (d, J=2.0 Hz, 1H), 7.45 (s, 1H), 7.44-7.40 (m, 2H), 7.39 (d, J=3.2 Hz, 1H), 7.38-7.35 (m, 2H), 7.34-7.30 (m, 2H), 7.27 (d, J=7.2 Hz, 1H), 4.96 (t, J=6.8 Hz, 1H), 4.20-4.17 (m, 1H), 3.93-3.85 (m, 1H), 3.80 (s, 2H), 3.77-3.66 (m, 2H), 3.66-3.58 (m, 2H), 3.57-3.42 (m, 5H), 3.38-3.33 (m, 2H), 3.28-3.22 (m, 2H), 2.97 (s, 2H), 2.89-2.80 (m, 2H), 2.79-2.69 (m, 5H), 2.55 (s, 2H), 2.44 (s, 2H), 2.25 (d, J=7.2 Hz, 2H), 2.18-2.09 (m, 3H), 2.08-2.00 (m, 1H), 1.77 (d, J=12.0 Hz, 2H), 1.63-1.51 (m, 1H), 1.33-1.24 (m, 5H), 1.11 (d, J=6.8 Hz, 3H).

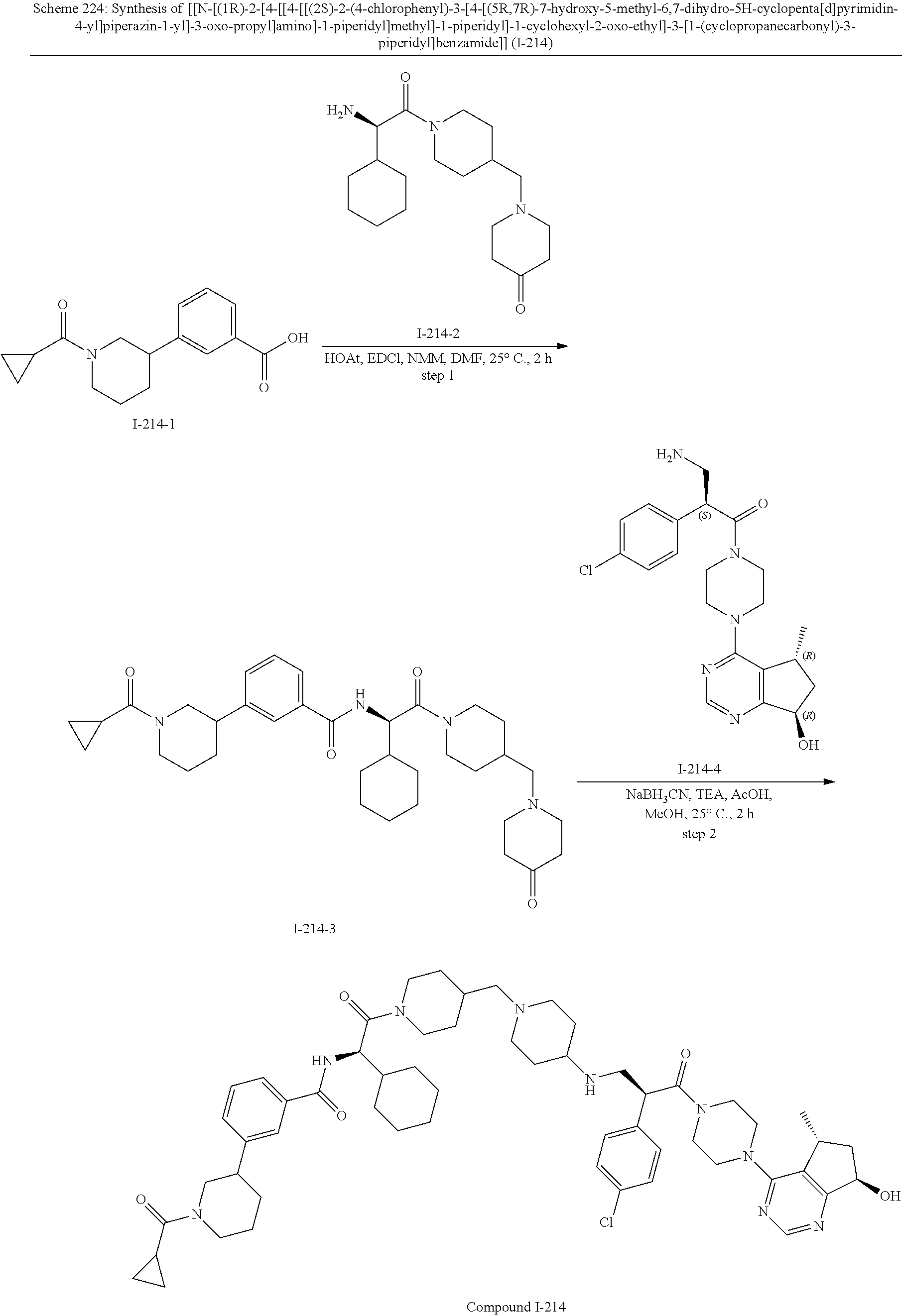

### Step 1: Synthesis of I-214-3

To a solution of I-214-1 (130 mg, 475.62 μmol, 1 eq) in DMF (1.5 mL) was added HOAt (129.47 mg, 951.24 μmol, 133.07 μL, 2 eq) NMM (481.08 mg, 4.76 mmol, 522.91 μL, 10 eq) and EDCI (455.89 mg, 2.38 mmol, 5 eq), then I-214-2 (191.48 mg, 570.75 μmol, 1.2 eq) was added in. The mixture was stirred at 25° C. for 2 hr. The residue was diluted with H₂O 5 mL and extracted with EA 15 mL (5 mL*3). The combined organic phase was dried over Na₂SO₄, filtered, and concentrated under reduced pressure to give a residue. The residue was purified by flash silica gel chromatography (ISCO®; 12 g SepaFlash® Silica Flash Column, Eluent of 0˜100% Dichloromethane/Methanol @ 60 mL/min). The eluent was concentrated under reduced pressure to give product. I-214-3 (200 mg, 338.53 μmol, 71.18% yield) was obtained as a white solid. LCMS (Method D): R^{t}=0.376 min, [M+H]⁺=591.3.

### Step 2: Synthesis of I-214

To a solution of I-214-4 (140.80 mg, 338.53 μmol, 1 eq) in MeOH (3 mL) was added TEA (205.53 mg, 2.03 mmol, 282.71 μL, 6 eq) stirred at 25° C. for 10 min, AcOH (121.98 mg, 2.03 mmol, 116.28 μL, 6 eq) and I-214-3 (200 mg, 338.53 μmol, 1 eq), then stirred at 25° C. for 10 min. Then NaBH₃CN (85.09 mg, 1.35 mmol, 4 eq) was added in. The mixture was stirred at 25° C. for 2 hr. The residue was diluted with H₂O 5 mL and extracted with EA 15 mL (5 mL*3) dried over Na₂SO₄, filtered, and concentrated under reduced pressure to give a residue. The crude product was purified by prep-HPLC (column: CD02-Waters Xbidge BEH C18 150*25*10 um; mobile phase: [water (NH₄HCO₃)-ACN]; gradient: 44%-74% B over 10 min). The eluent was lyophilized to give product which showed 88% purity. Then the product was repurified by reversed phase column (0.1% TFA condition). The eluent was concentrated under reduced pressure to remove ACN, then lyophilized to afford product. I-214 (30 mg, 27.03 μmol, 7.98% yield, 99.540% purity, TFA) was obtained as a white solid. LCMS (Method D): R^{t}=0.343 min, [M+H]⁺=990.4. ¹H NMR (400 MHz, METHANOL-d4) δ=8.64-8.55 (m, 1H), 7.85-7.65 (m, 2H), 7.55-7.41 (m, 4H), 7.37 (br d, J=8.4 Hz, 2H), 5.31 (t, J=8.0 Hz, 1H), 4.64-4.52 (m, 3H), 4.44-4.34 (m, 2H), 4.26-4.01 (m, 2H), 4.00-3.80 (m, 4H), 3.79-3.58 (m, 6H), 3.58-3.38 (m, 3H), 3.29-3.22 (m, 3H), 3.16-3.01 (m, 4H), 2.84-2.64 (m, 3H), 2.53-2.35 (m, 2H), 2.32-2.27 (m, 1H), 2.24-2.13 (m, 3H), 2.07 (br d, J=13.6 Hz, 2H), 1.88 (br d, J=13.6 Hz, 5H), 1.82-1.62 (m, 6H), 1.35-1.25 (m, 3H), 1.24-1.15 (m, 5H), 1.10 (br d, J=12.0 Hz, 2H), 0.89 (br d, J=4.4 Hz, 2H), 0.86-0.78 (m, 2H). ¹⁹F NMR (377 MHz, METHANOL-d4) δ=−77.142. SFC: R^{t}=3.533 min, 4.792 min.

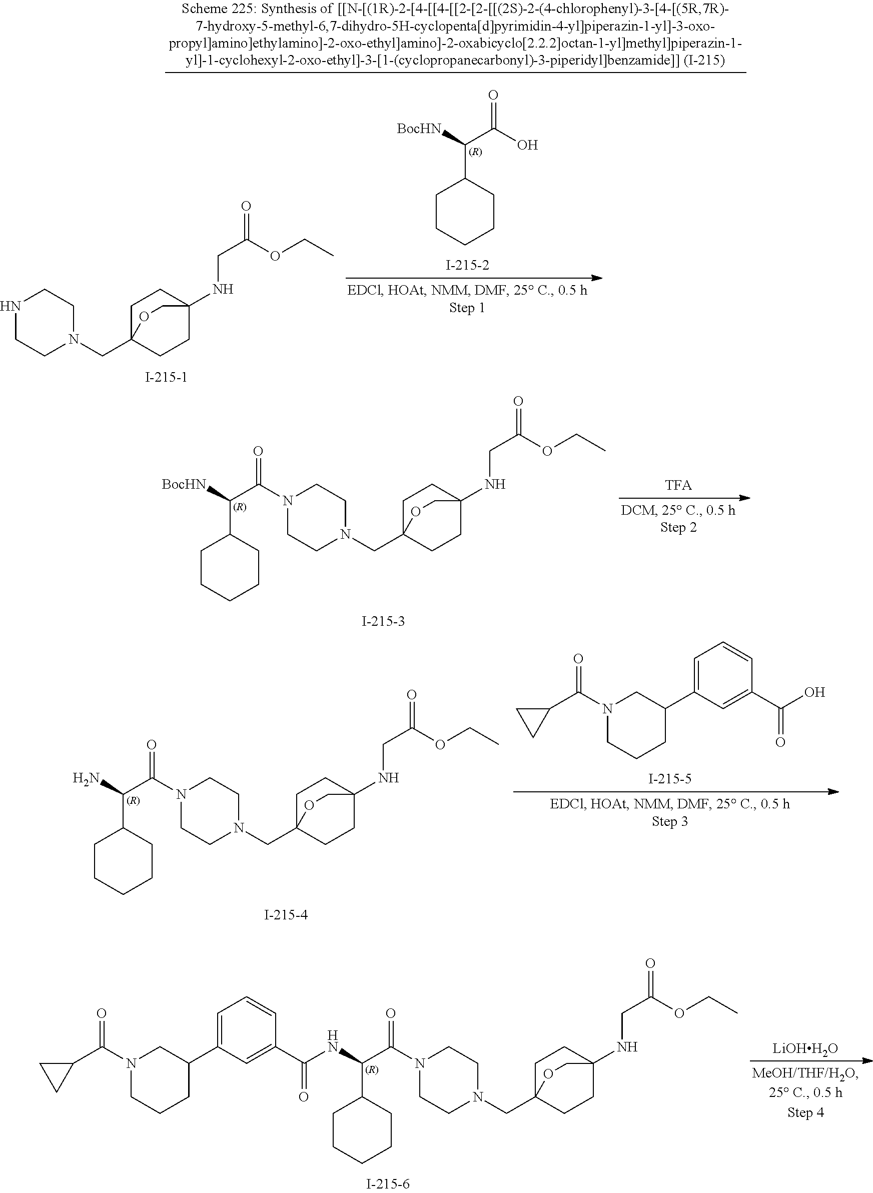

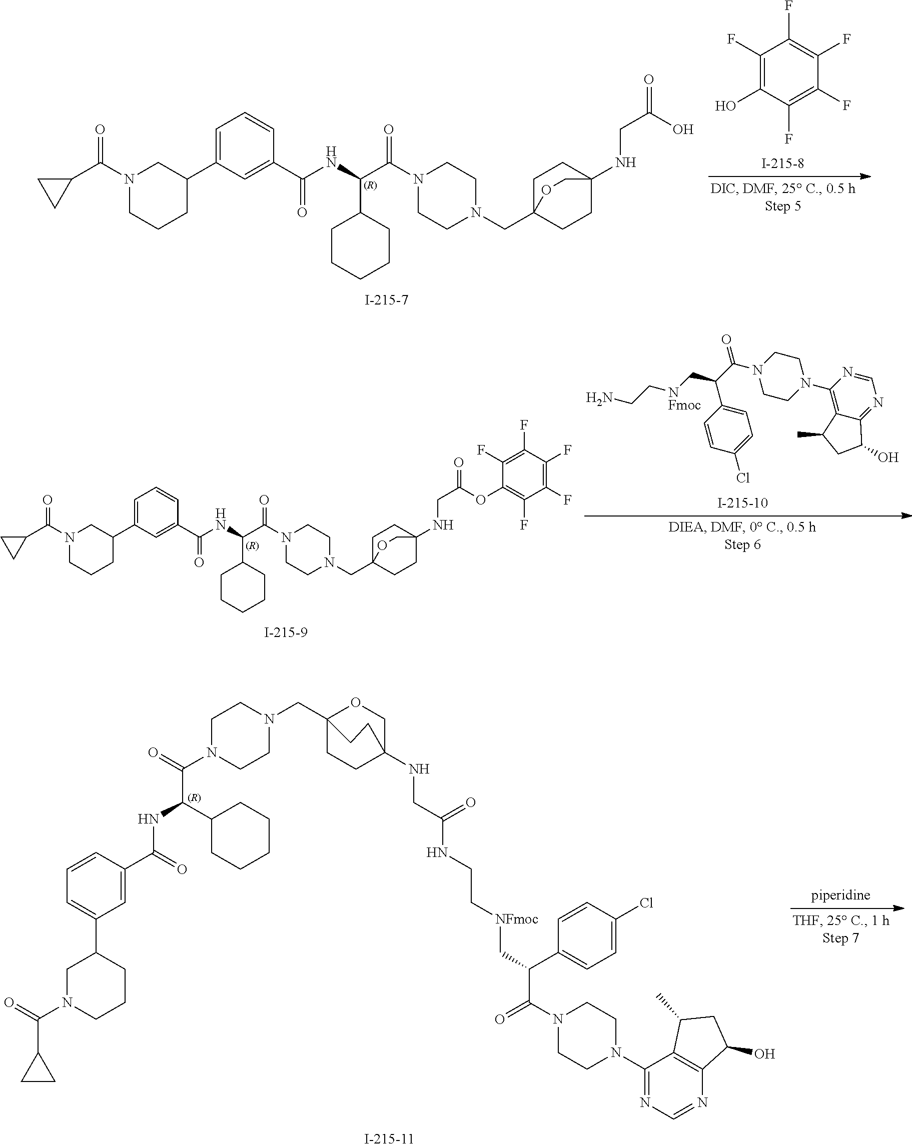

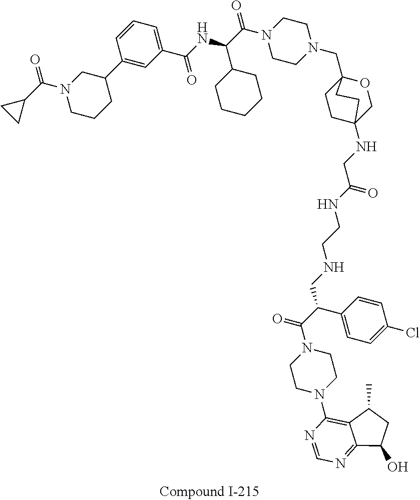 [SMILES: C[C@@H]1C[C@@H](O)c2ncnc(N3CCN(C(=O)[C@H](CNCCNC(=O)CNC45CCC(CN6CCN(C(=O)[C@H](NC(=O)c7cccc(C8CCCN(C(=O)C9CC9)C8)c7)C7CCCCC7)CC6)(CC4)OC5)c4ccc(Cl)cc4)CC3)c21]

### Step 1: Synthesis of I-215-3

To a solution of ethyl I-215-1 (200 mg, 642.22 μmol, 1 eq) and I-215-2 (198.31 mg, 770.67 μmol. 1.2 eq) in DMF (2 mL) was added EDCI (369.34 mg, 1.93 mmol, 3 eq), HOAt (87.41 mg, 642.22 μmol, 89.84 μL, 1 eq) and NMM (324.79 mg, 3.21 mmol, 353.04 μL, 5 eq). The mixture was stirred at 25° C. for 0.5 hr. The reaction mixture was concentrated under reduced pressure to give a residue. The residue was purified by reversed phase column (0.1% FA) and the eluent was concentrated to remove MeCN and then lyophilization. I-215-3 (300 mg, 544.73 μmol, 84.82% yield) was obtained as a yellow gum. LCMS: R^{t}=0.289 min, [M+H]⁺=551.4.

### Step 2: Synthesis of I-215-4

To a solution of I-215-3 (300 mg, 544.73 μmol, 1 eq) in DCM (3 mL) was added TFA (921.00 mg, 8.08 mmol, 0.6 mL, 14.83 eq). The mixture was stirred at 25° C. for 0.5 hr. The reaction mixture was concentrated under reduced pressure to give a product. I-215-4 (240 mg, crude, TFA) was obtained as a yellow gum. LCMS: R^{t}=0.258 min, [M+H]⁺=551.3.

### Step 3: Synthesis of I-215-6

To a solution of ethyl I-215-4 (220 mg, 488.22 μmol, 1 eq) and I-215-5 (133.44 mg, 488.22 μmol, 1 eq) in DMF (2 mL) was added EDCI (280.78 mg, 1.46 mmol, 3 eq), HOAt (66.45 mg, 488.22 μmol, 68.30 μL, 1 eq) and NMM (246.91 mg, 2.44 mmol, 268.38 μL, 5 eq). The mixture was stirred at 25° C. for 0.5 hr. The reaction mixture was poured into H₂O (5 mL), extract with EA (5 mL*3). The combined organic layers were dried over Na₂SO₄, filtered, and concentrated under reduced pressure to give a residue. The residue was purified by flash silica gel chromatography (ISCO®; 12 g SepaFlash® Silica Flash Column, Eluent of 0˜100% DCM/MeOH @ 36 mL/min, DCM/MeOH=10:1, Rf=0.3) and the eluent was concentrated under reduced pressure to give I-215-6 (230 mg, crude) as a yellow gum. LCMS (Method D): R^{t}=0.355 min, [M+H]⁺=706.5. SFC: R^{t}=1.203, 1.410 min.

### Step 4: Synthesis of I-215-7

To a solution of ethyl I-215-6 (230 mg, 325.81 μmol, 1 eq) in THF (1 mL), MeOH (1 mL) and H₂O (1 mL) was added LiOH·H₂O (41.01 mg, 977.44 μmol, 3 eq). The mixture was stirred at 25° C. for 0.5 hr. The reaction mixture was poured into H₂O (10 mL) at 25° C., the pH of the mixture was adjusted to 7 with 1M aqueous HCl, and then extracted with DCM 2 mL*3. The combined organic layers were dried over Na₂SO₄, filtered, and concentrated under reduced pressure to give a product. I-215-7 (200 mg, crude) was obtained as a yellow solid. LCMS: R^{t}==0.314 min, [M+H]⁺=678.3.

### Step 5: Synthesis of I-215-8

To a solution of I-215-7 (50 mg, 73.76 μmol, 1 eq) in DMF (0.5 mL) was added DIC (13.96 mg, 110.64 μmol, 17.13 μL, 1.5 eq) and I-215-8 (20.36 mg, 110.64 μmol, 1.5 eq). The mixture was stirred at 25° C. for 0.5 hr. The reaction mixture was concentrated under reduced pressure to give a product. I-215-9 (80 mg, crude) was obtained as a yellow gum. LCMS: R^{t}=0.358 min, [M+H]⁺=844.3.

### Step 6: Synthesis of I-215-11

To a solution of I-215-9 (70 mg, 82.95 μmol, 1 eq) and I-215-1 (59.53 mg, 87.39 μmol, 1.05 eq) in DMF (0.7 mL) was added DIEA (21.44 mg, 165.89 μmol, 28.90 μL, 2 eq). The mixture was stirred at 0° C. for 0.5 hr. The reaction mixture was concentrated under reduced pressure to give a residue. The residue was purified by reversed phase column (0.1% FA) and the eluent was concentrated to remove MeCN and then lyophilization. I-215-10 (40 mg, 29.83 μmol, 35.96% yield) was obtained as a yellow gum. LCMS: R^{t}=0.381 min, [M+H]⁺=671.3.

### Step 7: Synthesis of I-215

To a solution of I-215-11 (35 mg, 26.10 μmol, 1 eq) in THF (0.4 mL) was added PIPERIDINE (68.98 mg, 0.8 μmol, 0.08 mL, 31.039 eq). The mixture was stirred at 25° C. for 0.5 hr. The reaction mixture was concentrated under reduced pressure to give a residue. The residue was purified by reversed phase column (neutral condition). Then the eluent was concentrated to remove MeCN and then lyophilization. I-215 (11.65 mg, 10.41 μmol, 39.90% yield, 100% purity) was obtained as a white solid. Mass Found LCMS: R^{t}=0.301 min, [M+H]⁺=1118.5. SFC: R^{t}=3.380, 4.049 min. ¹H NMR (400 MHz, METHANOL-d4) δ=8.41 (s, 1H), 7.81-7.66 (m, 2H), 7.55-7.41 (m, 2H), 7.40-7.36 (m, 2H), 7.35-7.30 (m, 2H), 4.96 (t, J=6.8 Hz, 1H), 4.90 (s, 1H), 4.57 (d, J=10.8 Hz, 1H), 4.38 (d, J=5.4 Hz, 1H), 4.20-4.16 (m, 1H), 3.94-3.84 (m, 1H), 3.83-3.66 (m, 5H), 3.66-3.57 (m, 5H), 3.57-3.50 (m, 3H), 3.50-3.43 (m, 1H), 3.34 (s, 1H), 3.29-3.23 (m, 2H), 3.18 (s, 2H), 2.79-2.71 (m, 4H), 2.63-2.56 (m, 2H), 2.55-2.45 (m, 2H), 2.27 (s, 2H), 2.17-2.10 (m, 1H), 2.09-2.01 (m, 3H), 2.00-1.90 (m, 4H), 1.89-1.81 (m, 3H), 1.74 (d, J=11.2 Hz, 3H), 1.71-1.58 (m, 8H), 1.35-1.18 (m, 4H), 1.11 (d, J=6.8 Hz, 4H), 1.08 (s, 1H), 0.92-0.76 (m, 4H).

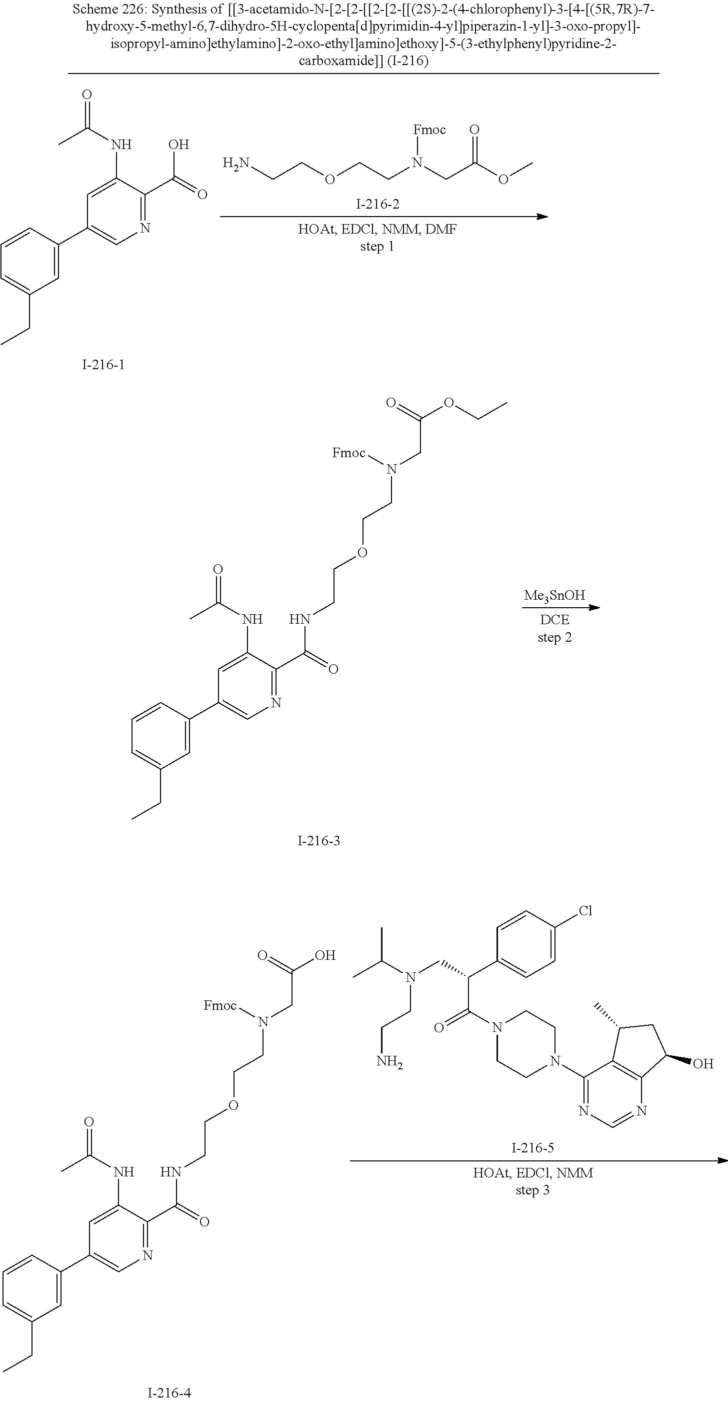

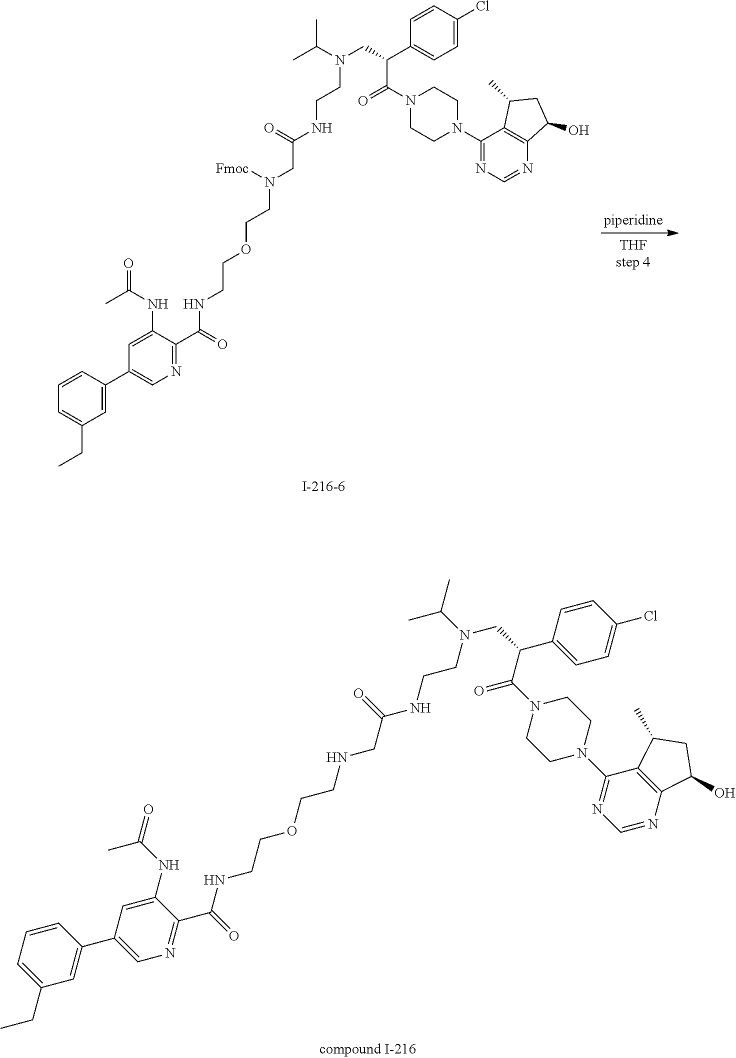

### Step 1: Synthesis of I-216-3.

To a solution of I-216-2 (201.59 mg, 439.67 μmol, 1.00 eq. FA) in DCM (3 mL) was added I-216-1 (125 mg, 439.66 μmol, 1 eq), HOAt (59.84 mg, 439.64 μmol, 61.50 μL, 1 eq), EDCI (252.86 mg, 1.32 mmol, 3.00 eq) and NMM (222.35 mg, 2.20 mmol, 241.68 μL, 5.00 eq). The mixture was stirred at 25° C. for 1 hr. The reaction mixture was diluted with H₂O (3 mL) and extracted with EA (3 mL*3). The combined organic layers were washed by brine (2 mL*3), dried over Na₂SO₄, filtered, and concentrated under reduced pressure to give a residue. The residue was purified by flash silica gel chromatography (ISCO®; 20 g SepaFlash® Silica Flash Column, Eluent of 35% EA/PE gradient (@, 50 mL/min). The eluent concentrated under reduced pressure to give I-216-3 (110 mg, 126.11 μmol, 28.68% yield, 77.817% purity) as a brown oil. LCMS (Method D): R^{t}=0.572 min, (M+H)=679.3. ¹H NMR (400 MHZ, CHLOROFORM-d) δ=7.71 (d, J=7.6 Hz, 1H), 7.65 (d, J=7.6 Hz, 1H), 7.61-7.48 (m, 2H), 7.48-7.37 (m, 4H), 7.36-7.17 (m, 6H), 4.44 (d, J=5.6 Hz, 1H), 4.36 (d, J=6.0 Hz, 1H), 4.19-4.09 (m, 2H), 4.08-4.00 (m, 3H), 3.96 (s, 1H), 3.63-3.55 (m, 3H), 3.54-3.47 (m, 3H), 2.82-2.53 (m, 3H), 2.26-2.18 (m, 3H), 1.29-1.25 (m, 3H), 1.22-1.14 (m, 3H).

### Step 2: Synthesis of I-216-4.

To a solution of I-216-3 (100 mg, 147.32 μmol, 1 eq) in DCE (2.5 mL) was added Me3SnOH (133.20 mg, 736.62 μmol, 5 eq). The mixture was stirred at 80° C. for 12 hr. The reaction mixture was diluted with H₂O (10 mL) and extracted with DCM (10 mL*3). The combined organic layers were dried over Na₂SO₄, filtered, and concentrated under reduced pressure to give a residue. The crude product was used for next step without further purification. I-216-4 (160 mg, crude) was obtained as a brown solid. LCMS (Method D): R^{t}=0.564 min, [M+H]′=651.2. ¹H NMR (400 MHZ, DMSO-d₆) δ=7.78 (d, J=7.6 Hz, 2H), 7.72 (d, J=7.6 Hz, 2H), 7.55-7.48 (m, 2H), 7.45-7.27 (m, 8H), 6.13 (s, 2H), 3.79 (s, 5H), 3.70-3.62 (m, 3H), 3.55 (d, J=5.2 Hz, 2H), 2.79-2.67 (m, 3H), 1.34-1.25 (m, 6H).

### Step 3: Synthesis of I-216-6.

To a solution of I-216-4 (120 mg, 184.41 μmol, 1 eq) in DMF (3 mL) was added HOAt (25.10 mg, 184.41 μmol, 25.80 μL, 1 eq), EDCI (106.06 mg, 553.23 μmol, 3 eq), NMM (93.26 mg, 922.06 μmol, 101.37 μL, 5 eq) and I-216-5 (113.43 mg, 184.41 μmol, 1.00 eq, TFA). The mixture was stirred at 25° C. for 1 hr. The reaction mixture was diluted with H₂O (10 mL) and extracted with EA (10 mL*3). The combined organic layers were washed by brine (10 mL*3), dried over Na₂SO₄, filtered, and concentrated under reduced pressure to give a residue. The residue was purified by flash silica gel chromatography (ISCO®; 20 g SepaFlash® Silica Flash Column, Eluent of 50% EA/PE gradient (@, 80 mL/min). The eluent concentrated under reduced pressure to give I-216-6 (72 mg, 59.28 μmol, 32.15% yield, 93.354% purity) as a white oil. LCMS (Method D): R^{t}=0.408 min, [M+H]⁺=1133.3. SFC: R^{t}=1.528 min. ¹H NMR (400 MHz, CHLOROFORM-d) δ=10.47-9.96 (m, 1H), 8.51-8.42 (m, 1H), 8.15 (s, 1H), 7.85-7.67 (m, 3H), 7.41-7.29 (m, 2H), 7.12-7.00 (m, 1H), 5.31 (d, J=8.0 Hz, 1H), 4.51-4.41 (m, 1H), 4.29 (s, 2H), 3.84-3.75 (m, 2H), 3.73-3.67 (m, 2H), 3.66-3.49 (m, 6H), 3.40-3.24 (m, 4H), 3.02-2.94 (m, 2H), 2.45 (d, J=16.8 Hz, 5H), 2.27-2.23 (m, 2H), 1.87-1.71 (m, 5H), 1.65-1.51 (m, 4H), 1.44 (s, 9H), 1.34-1.01 (m, 8H).

### Step 4: Synthesis of 1-216.

To a solution of I-216-6 (60 mg, 52.92 μmol, 1 eq) in THF (1 mL) was added piperidine (172.44 mg, 2.03 mmol, 0.2 mL, 38.27 eq). The mixture was stirred at 25° C. for 1 hr. The reaction mixture was concentrated under reduced pressure to give a residue. The residue was purified by reversed phase column (0.1% FA condition). The eluent was concentrated and lyophilized to give I-216 (19.96 mg, 20.08 μmol, 37.93% yield, 96.309% purity, FA) as a white solid. LCMS (Method D): R^{t}=0.377 min, [M+H]⁺=911.5. SFC: R^{t}=1.439 min. ¹H NMR (400 MHZ, CHLOROFORM-d) ¿=9.22 (s, 1H), 8.53 (s, 1H), 8.39 (s, 2H), 7.47 (d, J=13.6 Hz, 2H), 7.41-7.38 (m, 1H), 7.36-7.28 (m, 5H), 5.01-4.94 (m, 1H), 4.35 (s, 1H), 3.82-3.56 (m, 14H), 3.44 (d, J=6.8 Hz, 1H), 3.38-3.33 (m, 1H), 3.27-3.11 (m, 6H), 2.86-2.63 (m, 5H), 2.23 (s, 3H), 2.15-2.01 (m, 2H), 1.80-1.68 (m, 1H), 1.28-1.25 (m, 3H), 1.08 (s, 6H), 0.99 (d, J=6.0 Hz, 3H).

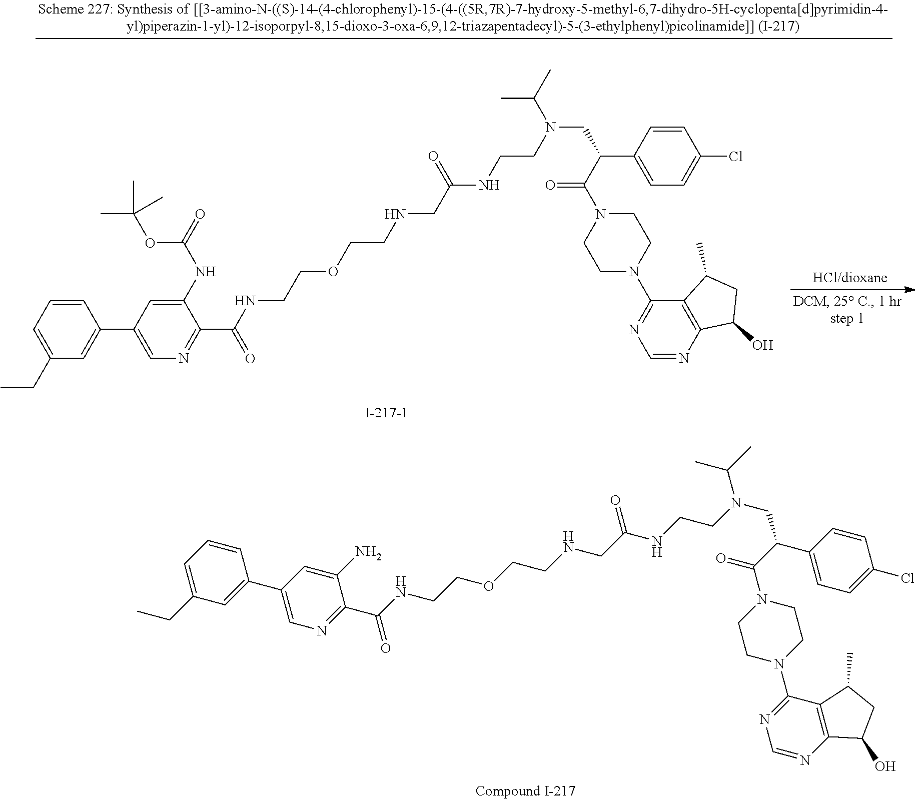 [SMILES: CCc1cccc(-c2cnc(C(=O)NCCOCCNCC(=O)NCCN(C[C@@H](C(=O)N3CCN(c4ncnc5c4[C@H](C)C[C@H]5O)CC3)c3ccc(Cl)cc3)C(C)C)c(NC(=O)OC(C)(C)C)c2)c1 ; CCc1cccc(-c2cnc(C(=O)NCCOCCNCC(=O)NCCN(C[C@@H](C(=O)N3CCN(c4ncnc5c4[C@H](C)C[C@H]5O)CC3)c3ccc(Cl)cc3)C(C)C)c(N)c2)c1]

### Step 1: Synthesis of I-217

To a solution of I-217-1 (40 mg, 41.25 μmol, 1 eq) in DCM (0.3 mL) was added HCl/dioxane (2 M, 0.3 mL). The mixture was stirred at 25° C. for 1 hr. The reaction mixture was concentrated in vacuo. The residue was purified by prep-HPLC (column: CD03-Welch Xtimate C18 150*25*5 um; mobile phase: [water (FA)-ACN]; gradient: 15%-45% B over 10 min), the eluent was concentrated under reduced pressure to remove ACN and lyophilized to give I-217 (29.88 mg, 32.47 μmol, 78.71% yield, 98.453% purity, HCl) was obtained as white solid. LCMS (Method D): R^{t}=0.311 min, (M+H)=869.4. SFC: R^{t}=1.738 min. ¹H NMR (400 MHZ, METHANOL-d4) δ=8.40 (s, 2H), 8.07 (d, J=1.6 Hz, 1H), 7.43 (s, 1H), 7.42-7.39 (m, 1H), 7.39-7.32 (m, 6H), 7.26 (d, J=7.2 Hz, 1H), 4.97 (m, 1H), 4.43 (m, 1H), 3.91-3.84 (m, 1H), 3.80 (m, 4H), 3.75-3.67 (m, 4H), 3.66-3.60 (m, 4H), 3.59 (s, 1H), 3.54-3.49 (m, 1H), 3.48-3.38 (m, 3H), 3.32 (s, 1H), 3.30-3.28 (m, 1H), 3.25 (d, J=4.4 Hz, 3H), 2.92-2.83 (m, 2H), 2.81-2.73 (m, 1H), 2.73-2.67 (m, 2H), 2.17-2.07 (m, 1H), 2.06-1.99 (m, 1H), 1.27 (m, 3H), 1.15-1.10 (m, 3H), 1.08 (d, J=6.8 Hz, 3H), 1.05 (d, J=6.8 Hz, 3H).

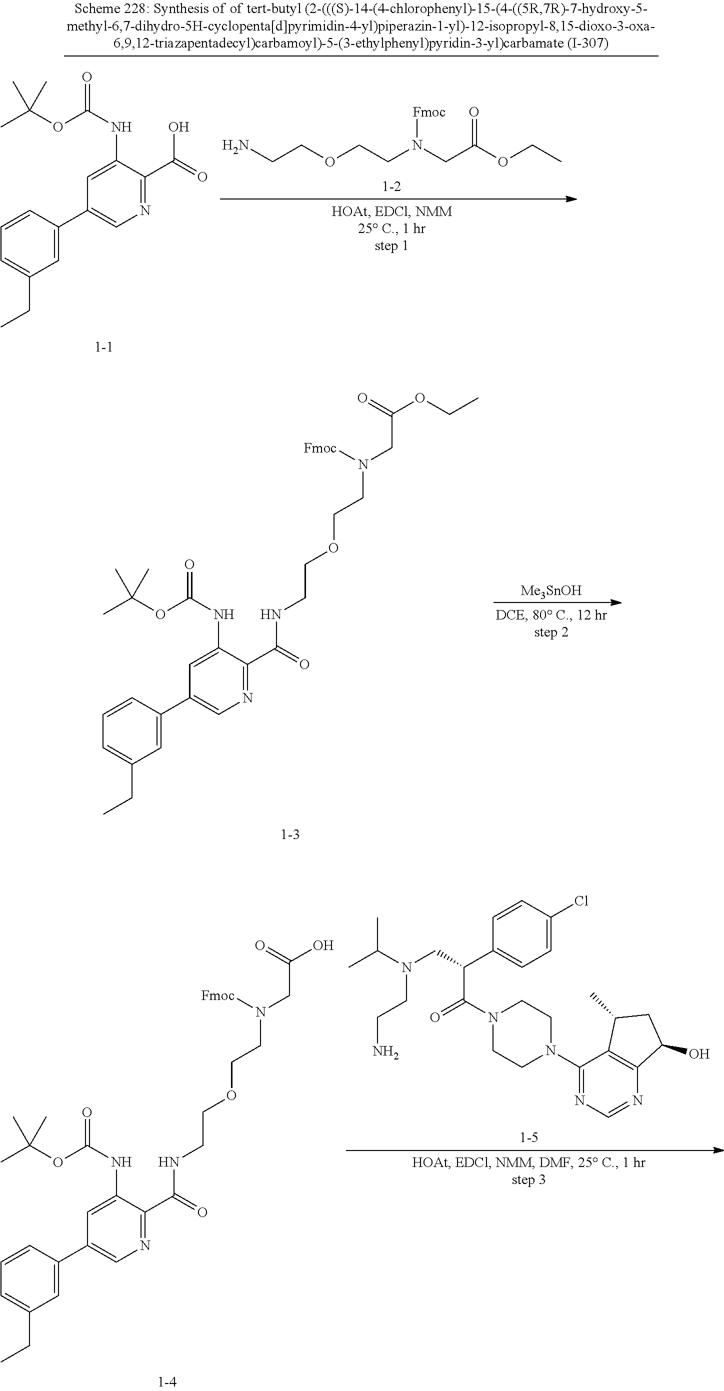

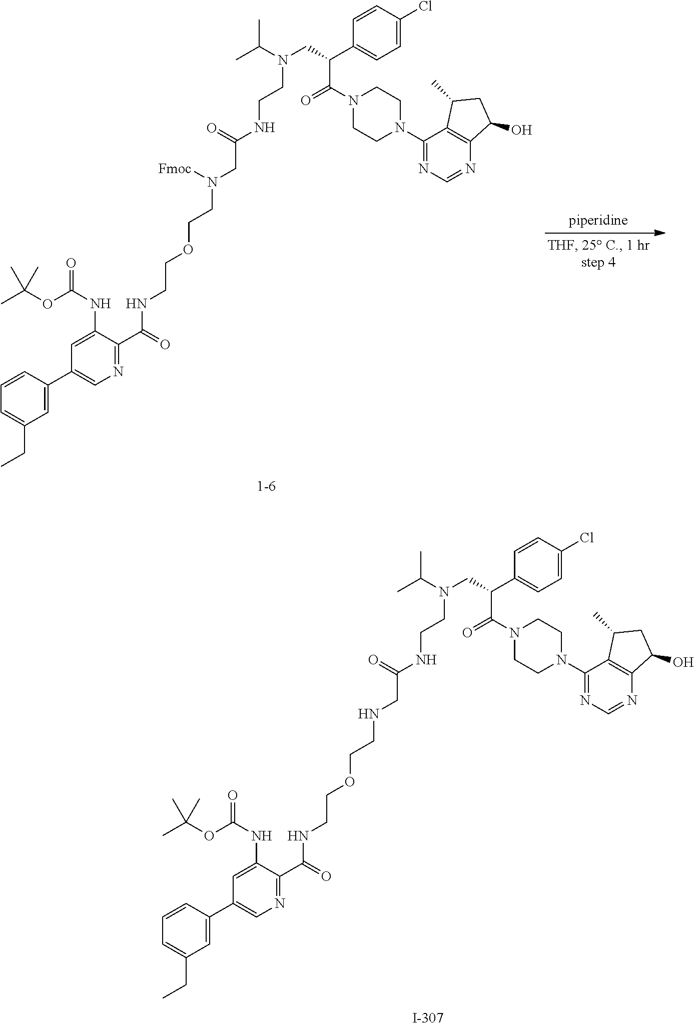

To a solution of 1-1 (150 mg, 438.10 μmol, 1 eq) in DMF (1.5 mL) was added HOAt (59.63 mg, 438.10 μmol, 61.28 μL, 1 eq), EDCI (251.95 mg, 1.31 mmol, 3 eq), NMM (221.56 mg, 2.19 mmol, 240.83 μL, 5 eq) and 1-2 (196.68 mg, 438.10 μmol, 1 eq, HCl). The mixture was stirred at 25° C. for 1 hr. The mixture was washed with water (1 mL) and extract with EA (2 mL*3). The combined organic layers dried over sodium sulfate, filtered, and concentrated under reduced pressure to give a residue. The residue was purified by reversed phase HPLC (FA condition), the eluent was concentrated under reduced pressure to remove ACN and lyophilized to give 1-3 (100 mg, 135.71 μmol, 30.98% yield, 100% purity) was obtained as white solid. LCMS (Method D): R^{t}=0.643 min, [M+H]⁺=737.4.

### Step 2: Synthesis of 1-4

To a solution of 1-3 (230 mg, 312.14 μmol, 1 eq) in DCE (2 mL) was added hydroxy (trimethyl) stannane (282.21 mg, 1.56 mmol, 5 eq). The mixture was stirred at 80° C. for 12 hr. The mixture was washed with aq. KF (10 mL) and diluted with water (5 mL). The mixture was stirred at 25° C. for 1 hr. Then the mixture was adjusted to pH=6˜7 with saturated citric acid aqueous solution. Then the reaction mixture was extracted with DCM (3 mL×3). The combined organic layers were washed with brine (3 mL×3) and dried over sodium sulfate, filtered, and concentrated under reduced pressure to give a residue. The residue was purified by flash silica gel chromatography (ISCO®; 12 g SepaFlash® Silica Flash Column, Eluent of 0˜50% EA/MeOH gradient @ 30 mL/min) and then the eluent was concentrated in vacuo to give 1-4 (100 mg, 139.82 μmol, 44.79% yield, 99.106% purity) was obtained as yellow oil. LCMS: R^{t}=0.597 min, [M+H]⁺=709.3.

### Step 3: Synthesis of 1-6

To a solution of 1-4 (80 mg, 112.87 μmol, 1 eq) in DMF (0.8 mL) was added HOAt (15.36 mg, 112.87 μmol, 15.79 μL, 1 eq), EDCI (64.91 mg, 338.60 μmol, 3 eq), NMM (57.08 mg, 564.34 μmol, 62.04 μL, 5 eq) and 1-5 (69.42 mg, 112.87 μmol, 1 eq, TFA). The mixture was stirred at 25° C. for 1 hr. The mixture was washed with water (1 mL) and extract with EA (2 mL*3). The combined organic layers dried over sodium sulfate, filtered, and concentrated under reduced pressure to give a residue. The residue was purified by reversed phase HPLC (FA condition), the eluent was concentrated under reduced pressure to remove ACN and lyophilized to give 1-6 (87 mg, 73.00 μmol, 64.67% yield, 100% purity) was obtained as white solid. LCMS (Method D): R^{t}=0.462 min, [M+H]⁺=1191.5. SFC: R^{t}=1.850 min.

### Step 4: Synthesis of I-307

To a solution of 1-6 (77 mg, 64.61 μmol, 1 eq) in THF (0.8 mL) was added piperidine (0.1 mL). The mixture was stirred at 25° C. for 1 hr. The reaction mixture was concentrated in vacuo. The residue was purified by reversed phase HPLC (FA condition), the eluent was concentrated under reduced pressure to remove ACN and lyophilized to give I-307 (35 mg, 36.10 μmol, 55.87% yield, 100% purity) was obtained as white solid. LCMS (Method D): R^{t}=0.380 min, [M+H]⁺=969.5. SFC: R^{t}: 1.388 min. ¹H NMR (400 MHz, METHANOL-d4) δ=9.01 (s, 1H), 8.47 (s, 2H), 8.39 (s, 1H), 7.53-7.47 (m, 2H), 7.41 (m, 1H), 7.33 (s, 5H), 4.95 (s, 1H), 4.23 (s, 1H), 3.91-3.82 (m, 1H), 3.69 (s, 6H), 3.64 (s, 6H), 3.52 (s, 3H), 3.46-3.40 (m, 1H), 3.23-3.09 (m, 3H), 3.01 (s, 3H), 2.73 (d, J=7.6 Hz, 3H), 2.58 (s, 1H), 2.49 (s, 1H), 2.15-2.00 (m, 2H), 1.54 (s, 9H), 1.28 (m, 3H), 1.09 (d, J=5.6 Hz, 3H), 0.99 (d, J=2.8 Hz, 3H), 0.91 (d, J=2.8 Hz, 3H).

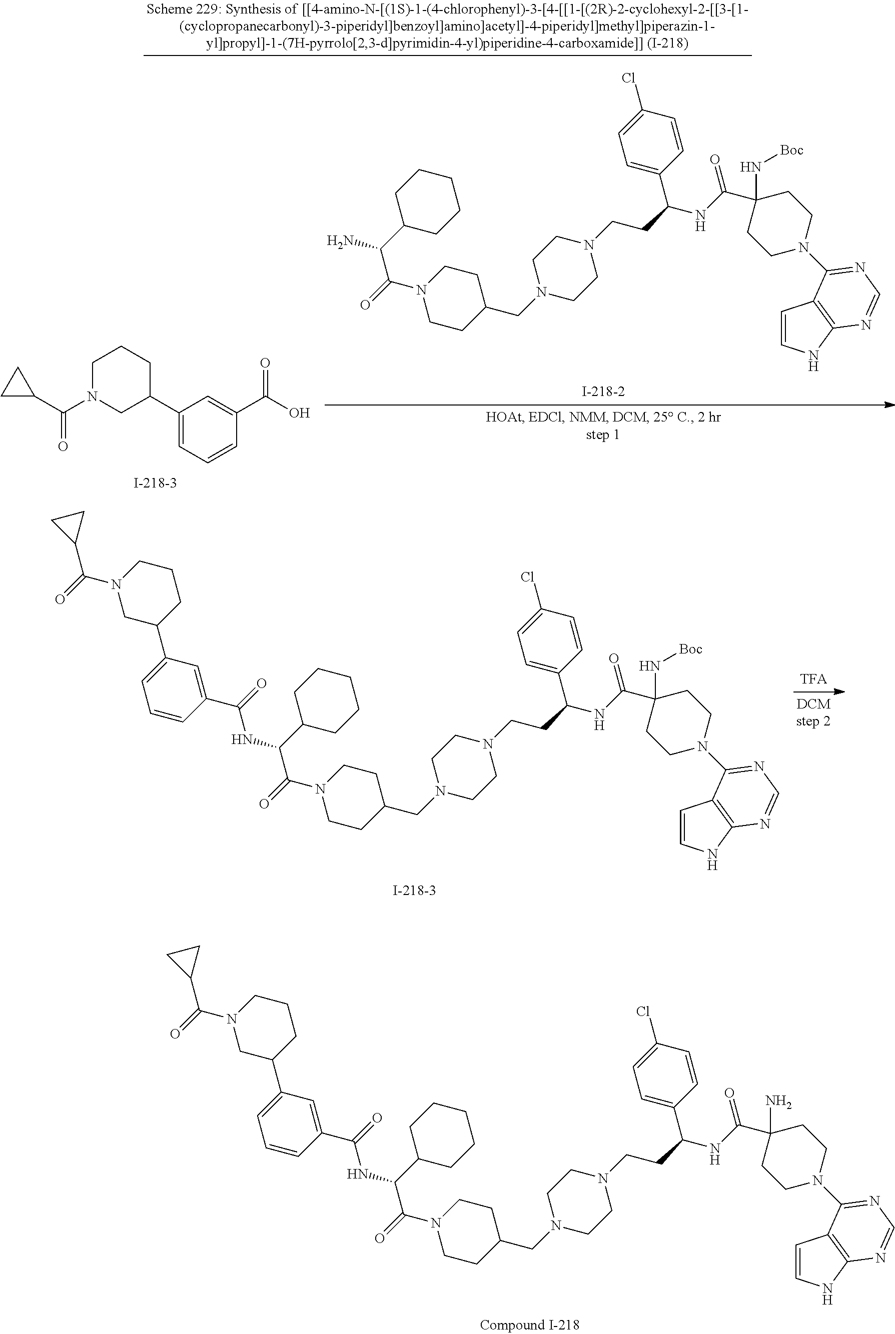 [SMILES: O=C(O)c1cccc(C2CCCN(C(=O)C3CC3)C2)c1 ; CC(C)(C)OC(=O)NC1(C(=O)N[C@@H](CCN2CCN(CC3CCN(C(=O)[C@H](N)C4CCCCC4)CC3)CC2)c2ccc(Cl)cc2)CCN(c2ncnc3[nH]ccc23)CC1 ; CC(C)(C)OC(=O)NC1(C(=O)N[C@@H](CCN2CCN(CC3CCN(C(=O)[C@H](NC(=O)c4cccc(C5CCCN(C(=O)C6CC6)C5)c4)C4CCCCC4)CC3)CC2)c2ccc(Cl)cc2)CCN(c2ncnc3[nH]ccc23)CC1 ; NC1(C(=O)N[C@@H](CCN2CCN(CC3CCN(C(=O)[C@H](NC(=O)c4cccc(C5CCCN(C(=O)C6CC6)C5)c4)C4CCCCC4)CC3)CC2)c2ccc(Cl)cc2)CCN(c2ncnc3[nH]ccc23)CC1]

### Step 1: Synthesis of I-218-3

To a solution of I-218-2 (11.48 mg, 41.99 μmol, 1 eq) in DCM (0.2 mL) was added EDCI (24.15 mg, 125.97 μmol, 3 eq), NMM (21.24 mg, 209.96 μmol, 23.08 μL, 5 eq) and HOAt (5.72 mg, 41.99 μmol, 5.87 μL, 1 eq), the mixture was stirred at 25° C. for 0.5 hr. And then I-218-1 (35 mg, 41.99 μmol, 1 eq) was added to the above mixture. The resulting mixture stirred at 25° C. for 0.5 hr. The mixture was concentrated under reduced pressure to give I-218-3 (40 mg, crude) as a yellow oil. LCMS: R^{t}=0.392 min, [M+H]⁺=1088.8.

### Step 2: Synthesis of I-218

To a solution of I-218-3 (30 mg, 27.55 μmol, 1 eq) in DCM (0.3 mL) was added TFA (60.00 μL). The mixture was stirred at 25° C. for 0.5 hr. The mixture was concentrated under reduced pressure to give a residue. The residue was purified by prep-HPLC (column: CD07-Daisogel SP-100-8-ODS-PK 150*25*10 um; mobile phase: [water (NH4HCO3)-ACN]; gradient: 43%-67% B over 12 min). Then the eluent was concentrated under reduced pressure to remove ACN and then lyophilized to give I-218 (5.16 mg, 5.09 μmol, 18.49% yield, 97.596% purity) as a white solid. LCMS (Method D): R^{t}=0.340 min, [M+H]⁺=988.4. ¹H NMR (400 MHZ, METHANOL-d4) δ=8.13 (s, 1H), 7.84-7.67 (m, 2H), 7.57-7.39 (m, 2H), 7.32 (s, 4H), 7.12 (s, 1H), 6.63 (s, 1H), 4.92 (s, 2H), 4.60-4.45 (m, 4H), 4.43-4.34 (m, 1H), 4.33-4.24 (m, 1H), 3.71-3.59 (m, 2H), 3.25-3.09 (m, 2H), 2.91-2.63 (m, 4H), 2.58-2.41 (m, 6H), 2.35 (d, J=4.8 Hz, 2H), 2.26-2.00 (m, 7H), 1.98-1.76 (m, 10H), 1.72-1.55 (m, 5H), 1.34-0.99 (m, 8H), 0.93-0.77 (m, 4H). SFC: R^{t}=0.960 min, 1.075 min.

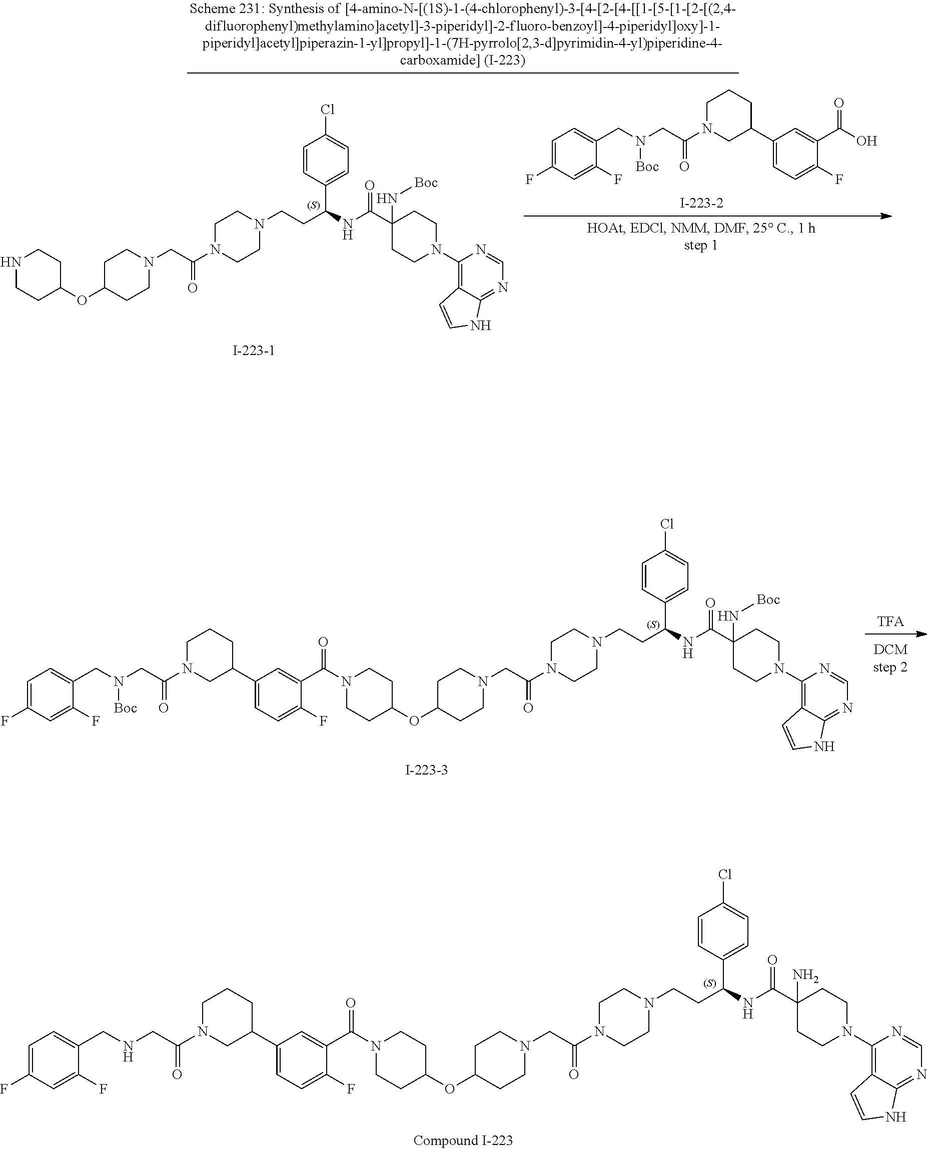

### Step 1: Synthesis of I-223-3

To a solution of I-223-2 (35 mg, 69.10 μmol, 1 eq) in DMF (0.5 mL) was added EDCI (39.74 mg, 207.30 μmol, 3 eq), HOAt (9.41 mg, 69.10 μmol, 9.67 μL, 1 eq), NMM (34.95 mg, 345.50 μmol, 37.99 μL, 5 eq) and I-223-1 (56.76 mg, 69.10 μmol, 1 eq). The mixture was stirred at 25° C. for 1 hr. The reaction was poured into water (5 mL) and extracted with EA (3 mL*3). The combined organic layers were dried over Na₂SO₄, filtered, and concentrated under reduced pressure to give a residue. The residue was purified by flash silica gel chromatography (ISCO®; 4 g SepaFlash® Silica Flash Column, Eluent of 0˜100% DCM/MeOH @ 18 mL/min, DCM/MeOH=10:1, Rf=0.2) and the eluent was concentrated under reduced pressure to give a residue. I-223-3 (60 mg, 45.80 μmol, 66.29% yield) was obtained as a yellow solid. LCMS (Method D): R^{t}=0.389 min, [M+23]⁺=1309.3.

### Step 2: Synthesis of 1-223

To a solution of I-223-3 (50 mg, 38.17 μmol, 1 eq) in DCM (0.5 mL) was added TFA (255.83 mg, 2.24 mmol, 166.67 μL, 58.78 eq). The mixture was stirred at 25° C. for 0.5 hr. The reaction mixture was combined with first batch EC3201-1155 for work-up, the resulting mixture was concentrated under reduced pressure to give a residue. The residue was purified by reversed phase column (0.1% TFA condition) and the eluent was lyophilized to give the product. I-223 (30.67 mg, 24.53 μmol, 64.26% yield, 97.862% purity, TFA) was obtained as a white solid. LCMS (Method D): R^{t}=0.264 min, [M+H]⁺=1109.4. ¹H NMR (400 MHz, METHANOL-d4) δ=8.35 (s, 1H), 7.64-7.58 (m, 1H), 7.48-7.41 (m, 1H), 7.37-7.34 (m, 5H), 7.31 (d, J=6.4 Hz, 1H), 7.17 (s, 1H), 7.15-7.07 (m, 2H), 6.88 (d, J=3.6 Hz, 1H), 5.03 (t, J=7.6 Hz, 1H), 4.69-4.49 (m, 4H), 4.37-4.27 (m, 5H), 4.27-4.20 (m, 1H), 4.18 (d, J=10.4 Hz, 1H), 4.09 (d, J=16.0 Hz, 1H), 3.96-3.90 (m, 1H), 3.81 (d, J=2.0 Hz, 5H), 3.71 (br s, 4H), 3.55-3.44 (m, 3H), 3.24 (d, J=1.6 Hz, 2H), 3.16-3.10 (m, 2H), 2.92-2.77 (m, 2H), 2.74 (s, 1H), 2.65-2.52 (m, 2H), 2.38-2.26 (m, 2H), 2.16 (d, J=14.8 Hz, 2H), 2.10-1.97 (m, 6H), 1.94-1.77 (m, 5H), 1.74-1.48 (m, 4H). ¹⁹F NMR (400 MHz, METHANOL-d4) δ=−76.286, −118.403, −112.891, −120.532. SFC: R^{t}=4.669, 5.787 min.

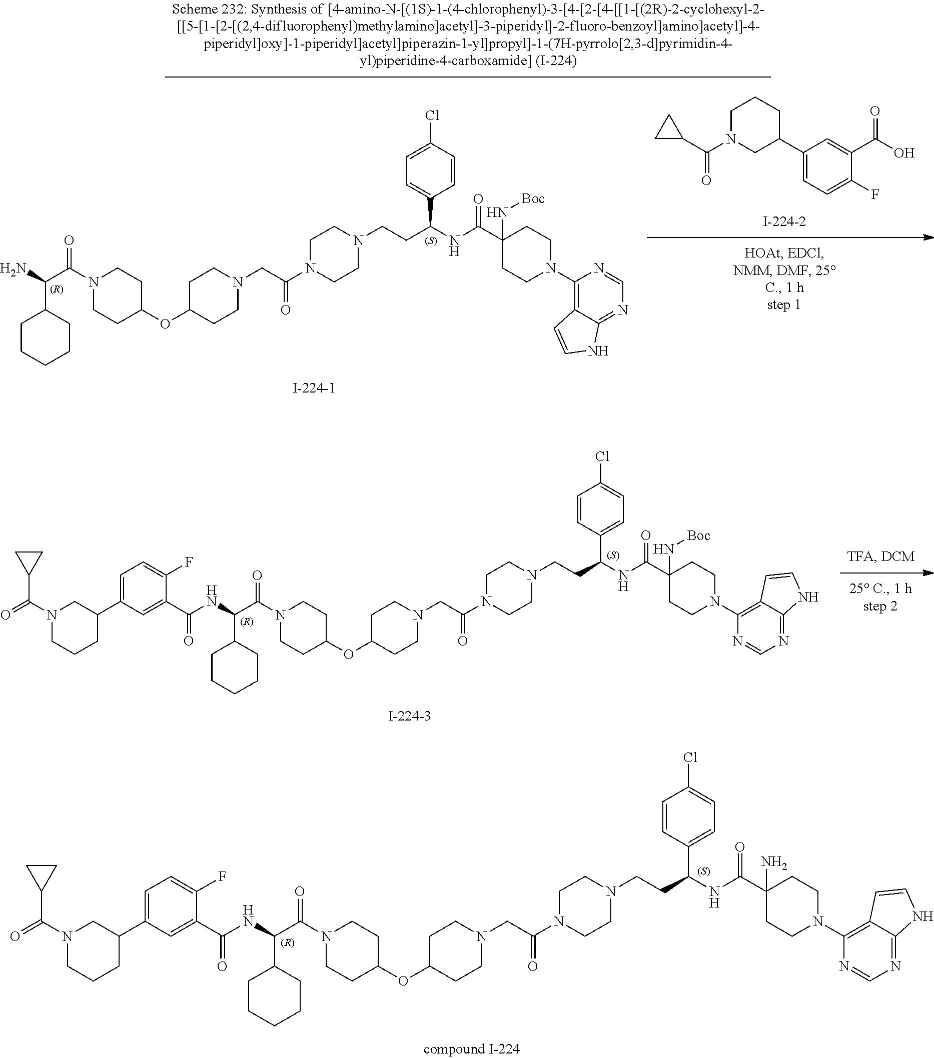

### Step 1: Synthesis of I-224-3

To a solution of I-224-2 (39.54 mg, 78.07 μmol, 1.5 eq) in DMF (0.5 mL) was added EDCI (29.93 mg, 156.15 μmol, 3 eq), HOAt (7.08 mg, 52.05 μmol, 7.28 μL, 1 eq), NMM (26.32 mg, 260.24 μmol, 28.61 μL, 5 eq) and I-224-1 (50 mg, 52.05 μmol, 1 eq). The mixture was stirred at 25° C. for 1 hr. The reaction was poured into water (5 mL) and extracted with EA (3 mL*3). The combined organic layers were dried over Na₂SO₄, filtered, and concentrated under reduced pressure to give a residue. The residue was purified by flash silica gel chromatography (ISCO®; 4 g SepaFlash® Silica Flash Column, Eluent of 0˜100% DCM/MeOH @ 18 mL/min, DCM/MeOH=10:1, Rf=0.2) and the eluent was concentrated under reduced pressure to give a residue. I-224-3 (60 mg, crude) was obtained as a yellow solid LCMS (Method D): R^{t}=0.355 min, [M+23]⁺=1233.5.

### Step 2: Synthesis of I-224

To a solution of I-224-3 (60 mg, 48.62 μmol, 1 eq) in DCM (0.6 mL) was added TFA (307.00 mg, 2.69 mmol, 0.2 mL, 55.37 eq). The mixture was stirred at 25° C. for 0.5 hr. The mixture was concentrated under reduced pressure to give a residue. The residue was purified by prep-HPLC (column: CD01-Phenomenex luna C18 150*25*10 um; mobile phase: [water (TFA)-ACN]; gradient: 20%-50% B over 10 min) and concentrated under reduced pressure to remove ACN, and then lyophilized to give product. I-224 (28.1 mg, 22.33 μmol, 45.93% yield, 99.178% purity, TFA) was obtained as a white solid. LCMS (Method D): R^{t}=0.337 min, [M+H]⁺=1133.4. ¹H NMR (400 MHZ, METHANOL-d4) δ=8.37 (s, 1H), 7.71-7.59 (m, 1H), 7.55-7.44 (m, 1H), 7.36 (s, 5H), 7.25-7.14 (m, 1H), 6.92 (d, J=3.6 Hz, 1H), 5.03 (t, J=6.8 Hz, 1H), 4.99-4.94 (m, 1H), 4.69-4.60 (m, 2H), 4.54 (d, J=9.6 Hz, 1H), 4.37 (s, 1H), 4.30 (d, J=7.6 Hz, 2H), 4.10-3.89 (m, 3H), 3.88-3.74 (m, 5H), 3.74-3.61 (m, 3H), 3.58-3.42 (m, 3H), 3.41-3.34 (m, 3H), 3.28-3.20 (m, 2H), 3.15-3.12 (m, 2H), 2.88-2.75 (m, 1H), 2.75-2.67 (m, 2H), 2.66-2.53 (m, 2H), 2.42-2.26 (m, 2H), 2.17 (d, J=14.4 Hz, 2H), 2.11-1.98 (m, 6H), 1.96 (s, 2H), 1.89-1.74 (m, 7H), 1.70 (d, J=10.8 Hz, 4H), 1.59-1.52 (m, 1H), 1.35-1.21 (m, 3H), 1.19-1.10 (m, 2H), 0.91-0.76 (m, 4H). ¹⁹F NMR (400 MHZ, METHANOL-d4) δ=−77.089, −118.603. SFC: R^{t}=2.438, 3.137 min.

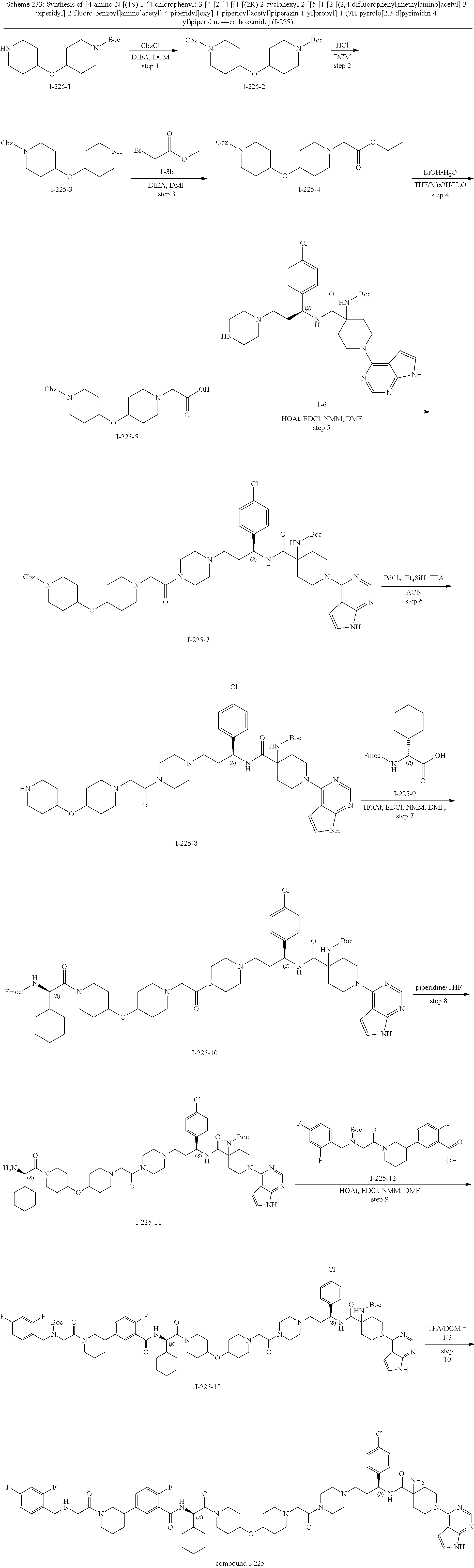

### Step 1: Synthesis of I-225-2

To a solution of I-225-1 (10 g, 35.16 mmol, 1 eq) and DIEA (9.09 g, 70.33 mmol, 12.25 mL, 2 eq) in DCM (100 mL) was added benzyl carbonochloridate (9.00 g, 52.74 mmol, 7.53 mL, 1.5 eq). The mixture was stirred at 25° C. for 1 hr. The mixture was concentrated under reduced pressure to give a residue. The residue was purified by flash silica gel chromatography (ISCO®; 120 g SepaFlash® Silica Flash Column, Eluent of 0˜100% PE/EA @ 100 mL/min, PE/EA=1:1, Rf=0.6) and the eluent was concentrated under reduced pressure to give a residue. I-225-2 (16 g, 30.58 mmol, 86.98% yield, 80% purity) was obtained as a yellow oil. LCMS: R^{t}=0.491 min, [M+Na⁺]=441.5. ¹H NMR (400 MHZ, METHANOL-d4) δ=7.39-7.32 (m, 5H), 5.12 (s, 2H), 3.85-3.65 (m, 6H), 3.26 (s, 2H), 3.20-3.08 (m, 2H), 1.85-1.81 (m, 4H), 1.54-1.40 (m, 13H).

### Step 2: Synthesis of I-225-3

To a solution of I-225-2 (14 g, 33.45 mmol, 1 eq) in DCM (20 mL) was added HCl/dioxane (2 M, 140 mL, 8.37 eq). The mixture was stirred at 25° C. for 0.5 hr. The mixture was concentrated under reduced pressure to give a residue. I-225-3 (14 g, crude, HCl) was obtained as a white solid. LCMS (Method D): R^{t}=0.315 min, [M+H]⁺=319.5.

### Step 3: Synthesis of I-225-4

To a solution of I-225-3 (12.5 g, 35.22 mmol, 1 eq, HCl) and I-225-3b (5.93 g, 38.75 mmol, 3.67 mL, 1.1 eq) in DMF (125 mL) was added DIEA (9.10 g, 70.45 mmol, 12.27 mL, 2 eq). The mixture was stirred at 25° C. for 1 hr. The reaction was poured into water (400 mL) and extracted with EA (200 mL*5). The combined organic layers were dried over Na₂SO₄, filtered, and concentrated under reduced pressure to give a residue. I-225-4 (15 g, crude) was obtained as a yellow oil. LCMS (Method D): R^{t}=0.295 min, [M+H]⁺=391.2.

### Step 4: Synthesis of I-225-5

To a solution of I-225-4 (2 g, 5.12 mmol, 1 eq) in THF (6 mL), MOH (6 mL) and H₂O (6 mL) was added LiOH·H₂O (644.81 mg, 15.37 mmol, 3 eq). The mixture was stirred at 25° C. for 0.5 hr. The reaction phase pH was adjusted to pH=7 with 1M HCl, The mixture was added H₂O (50 mL) and then extracted with CHCl₃/i-PrOH=3/1 (30 mL*5), dried by Na₂SO₄, filtered, and concentrated to give crude product. I-225-5 (2 g, crude) was obtained as a yellow oil. LCMS (Method D): R^{t}=0.158 min, [M+H]⁺=376.2.

### Step 5: Synthesis of I-225-7

To a solution of I-225-5 (2 g, 5.31 mmol, 1 eq) in DMF (30 mL) was added EDCI (3.06 g, 15.94 mmol, 3 eq), HOAt (723.14 mg, 5.31 mmol, 743.20 μL, 1 eq), NMM (2.69 g, 26.56 mmol, 2.92 mL, 5 eq) and I-225-6 (3.17 g, 5.31 mmol, 1 eq). The mixture was stirred at 25° C. for 1 hr. The reaction was poured into water (100 mL) and extracted with EA (60 mL*3). The combined organic layers were dried over Na₂SO₄, filtered, and concentrated under reduced pressure to give a residue. The residue was purified by flash silica gel chromatography (ISCO®; 40 g SepaFlash® Silica Flash Column, Eluent of 0˜100% DCM/MeOH @ 60 mL/min, DCM/MeOH=10:1, Rf=0.3) and the eluent was concentrated under reduced pressure to give a residue. I-225-7 (5 g. 5.23 mmol, 98.49% yield) was obtained as a white solid. LCMS (Method D): R^{t}=0.323 min, [M+H]⁺=955.5.

### Step 6: Synthesis of I-225-8

To a solution of I-225-7 (5 g, 5.23 mmol, 1 eq) in DCM (50 mL) was added PdCl2 (463.92 mg, 2.62 mmol, 0.5 eq), TEA (1.06 g, 10.46 mmol, 1.46 mL, 2 eq) and Et3SiH (4.87 g, 41.86 mmol, 6.69 mL, 8 eq). The mixture was stirred at 25° C. for 12 hr. The resulting reaction was adjusted to pH=8 with NH3·H₂O. The mixture was added H₂O (300 mL) and then extracted with DCM/MeOH=10/1 (200 mL*5), dried by Na₂SO₄, filtered, and concentrated to give crude product. I-225-8 (5 g, crude) was obtained as a yellow oil. LCMS (Method D): R^{t}=0.251 min, [M+H]⁺=821.5.

### Step 7: Synthesis of I-225-10

To a solution of I-225-9 (1.5 g, 3.95 mmol, 1.50 mL, 1 eq) in DMF (30 mL) was added EDCI (2.27 g. 11.86 mmol, 3 eq), HOAt (538.06 mg, 3.95 mmol, 552.99 μL, 1 eq), NMM (2.00 g, 19.77 mmol, 2.17 mL, 5 eq) and I-225-8 (3.25 g, 3.95 mmol, 1 eq). The mixture was stirred at 25° C. for 0.5 hr. The reaction was poured into water (100 mL) and extracted with EA (60 mL*3). The combined organic layers were dried over Na₂SO₄, filtered, and concentrated under reduced pressure to give a residue. The residue was purified by flash silica gel chromatography (ISCO®; 80 g SepaFlash® Silica Flash Column, Eluent of 0˜100% DCM/MeOH @ 100 mL/min, DCM/MeOH=10:1, Rf=0.2) and the eluent was concentrated under reduced pressure to give a residue. I-225-10 (3.5 g, 2.81 mmol, 71.11% yield, 95% purity) was obtained as a white solid. LCMS (Method D): R^{t}=0.357 min, [M+H]⁺=1182.5. SFC: R^{t}=2.083 min.

### Step 8: Synthesis of I-225-11

To a solution of I-225-10 (500 mg, 422.70 μmol, 1 eq) in THF (5 mL) was added PIPERIDINE (862.20 mg, 10.13 mmol, 1 mL, 23.96 eq). The mixture was stirred at 25° C. for 1 hr. Added PE (20 mL) to the reaction mixture, filtered, and the residue were used for purification. The residue was triturated with PE (3 mL) at 25° C. for 10 mins, the mixture was filtered, and the residue was concentrated under reduced pressure to give product. I-225-11 (400 mg, 383.08 μmol, 90.63% yield, 92% purity) was obtained as a white solid. LCMS (Method D): R^{t}=0.284 min, [M+H]⁺=960.5.

### Step 9: Synthesis of I-225-13

To a solution of I-225-12 (39.54 mg, 78.07 μmol, 1.5 eq) in DMF (0.5 mL) was added EDCI (29.93 mg, 156.15 μmol, 3 eq), HOAt (7.08 mg, 52.05 μmol, 7.28 μL, 1 eq), NMM (26.32 mg, 260.24 μmol, 28.61 μL, 5 eq) and I-225-11 (50 mg, 52.05 μmol, 1 eq). The mixture was stirred at 25° C. for 1 hr. The reaction was poured into water (5 mL) and extracted with EA (3 mL*3). The combined organic layers were dried over Na₂SO₄, filtered, and concentrated under reduced pressure to give a residue The residue was purified by flash silica gel chromatography (ISCO®; 4 g SepaFlash® Silica Flash Column, Eluent of 0˜100% DCM/MeOH @ 18 mL/min, DCM/MeOH=10:1, Rf=0.2) and the eluent was concentrated under reduced pressure to give a residue. I-225-13 (60 mg, 37.26 μmol, 71.59% yield) was obtained as a yellow solid. LCMS (Method D): R^{t}=0.403 min, [M+H]⁺=1148.6.

### Step 10: Synthesis of I-225

To a solution of I-225-13 (50 mg, 34.50 μmol, 1 eq) in DCM (0.5 mL) was added TFA (255.83 mg, 2.24 mmol, 166.67 μL, 65.03 eq). The mixture was stirred at 25° C. for 1 hr. The reaction mixture was combined with first batch EC3201-1156 for work-up, the resulting mixture was concentrated under reduced pressure to give a residue. The residue was purified by reversed phase column (TFA (0.1%) condition) and the eluent was lyophilized to give the product. I-225 (37.05 mg, 27.18 μmol, 78.79% yield, 100% purity, TFA) was obtained as a white solid. LCMS (Method D): R^{t}=0.317 min, [M+H]⁺=1248.4. ¹H NMR (400 MHz, METHANOL-d4) δ=8.40 (s, 1H), 7.73-7.59 (m, 2H), 7.57-7.47 (m, 1H), 7.41-7.35 (m, 5H), 7.28-7.20 (m, 1H), 7.19-7.08 (m, 2H), 6.95 (d, J=3.6 Hz, 1H), 5.07-4.99 (m, 2H), 4.69-4.62 (m, 2H), 4.59-4.49 (m, 1H), 4.36-4.31 (m, 4H), 4.27-4.21 (m, 1H), 4.20-4.13 (m, 1H), 4.06-3.96 (m, 2H), 3.92-3.79 (m, 5H), 3.77-3.65 (m, 4H), 3.56-3.53 (m, 1H), 3.52-3.39 (m, 4H), 3.25-3.13 (m, 3H), 3.06-2.70 (m, 3H), 2.70-2.54 (m, 2H), 2.44-2.29 (m, 2H), 2.20 (d, J=14.8 Hz, 2H), 2.07 (s, 6H), 1.97-1.77 (m, 9H), 1.77-1.44 (m, 6H), 1.39-1.07 (m, 6H). ¹⁹F NMR (377 MHz, METHANOL-d4) δ=−77.097, —108.403, −112.928, −118.182. SFC: R^{t}=2.999, 3.522 min.

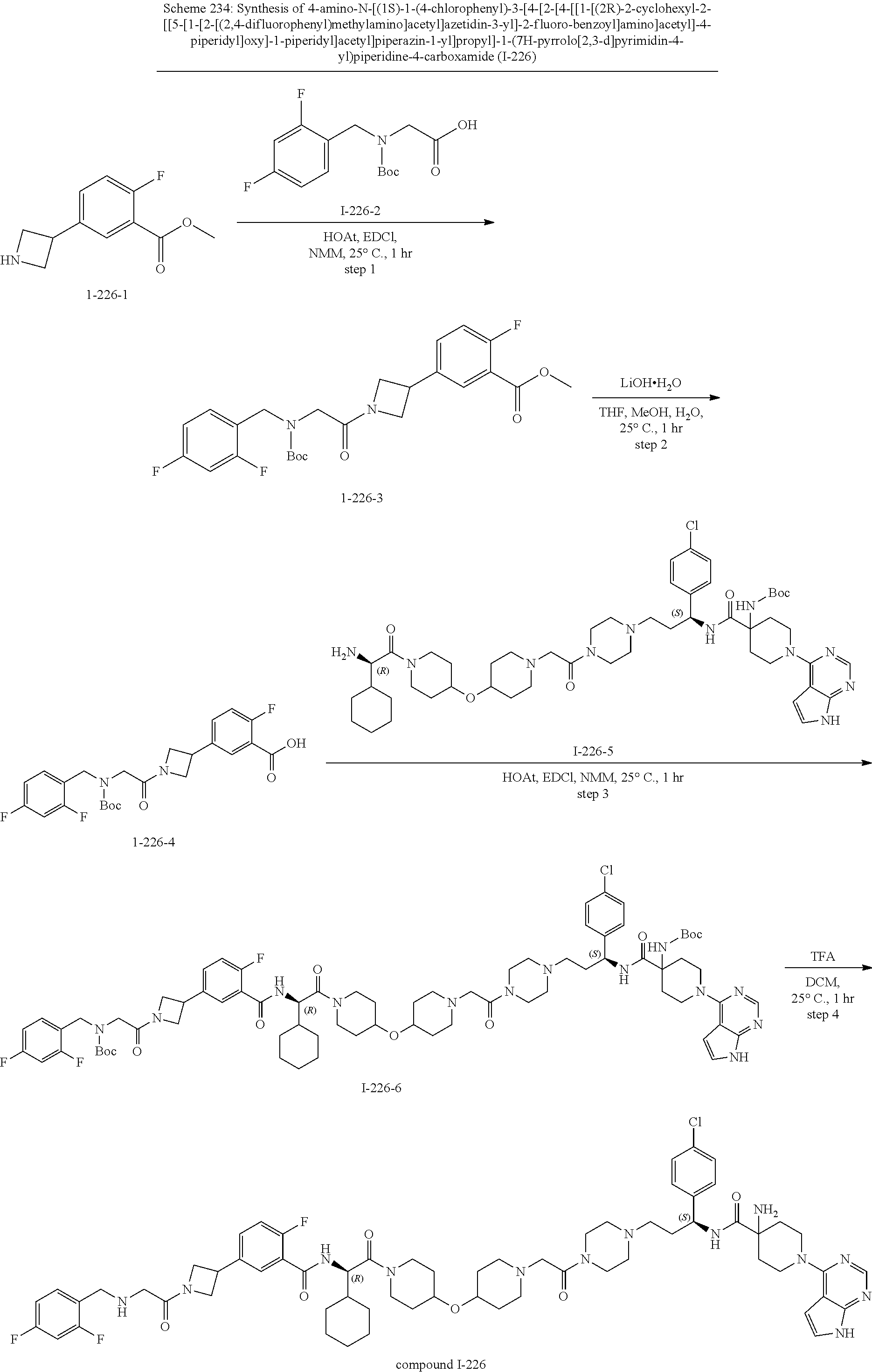

### Step 1: Synthesis of I-226-3

To a solution of I-226-1 (500 mg, 2.39 mmol, 1 eq) and I-226-2 (720.03 mg, 2.39 mmol, 1 eq) in DMF (5 mL) was added EDCI (1.37 g, 7.17 mmol, 3 eq), NMM (1.21 g, 11.95 mmol, 1.31 mL, 5 eq) and HOAt (325.29 mg, 2.39 mmol, 334.31 μL, 1 eq). The mixture was stirred at 25° C. for 1 hr. The reaction mixture was extracted with DCM (15 mL*3). The combined organic layers were dried over Na₂SO₄, filtered, and concentrated under reduced pressure to give a residue. The residue was purified by flash silica gel chromatography (ISCO®; 20 g SepaFlash® Silica Flash Column, Eluent of 0˜78% Ethylacetate/Petroleum ether gradient @ 36 mL/min) and concentrated under reduced pressure to give a residue. I-226-3 (1.2 g, crude) was obtained as a yellow oil. LCMS (Method D): R^{t}=0.465 min, [M+H]⁺=515.2.

### Step 2: Synthesis of I-226-4

To a solution of I-226-3 (1 g, 2.03 mmol, 1 eq) in MeOH (10 mL), THF (10 mL) and H₂O (5 mL) was added LiOH·H₂O (426.04 mg, 10.15 mmol, 5 eq). The mixture was stirred at 25° C. for 1 hr. The reaction mixture was concentrated under reduced pressure and adjusted pH=4 with HCl (1 M). Then the mixture was extracted with DCM (15 mL*3). The combined organic layers were dried over Na₂SO₄, filtered, and concentrated under reduced pressure to give product. I-226-4 (735 mg, 1.54 mmol, 75.65% yield) was obtained as a white solid. LCMS (Method D): R^{t}=0.435 min, [M+H]⁺=501.0.

### Step 3: Synthesis of I-226-6

To a solution of I-226-4 (40 mg, 83.60 μmol, 1.5 eq) and I-226-5 (53.54 mg, 55.73 μmol, 1 eq) in DMF (0.4 mL) was added EDCI (32.05 mg, 167.20 μmol, 3 eq), NMM (28.19 mg, 278.67 μmol, 30.64 μL, 5 eq) and HOAt (7.59 mg, 55.73 μmol, 7.80 μL, 1 eq). The mixture was stirred at 25° C. for 1 hr. The reaction mixture was extracted with DCM (5 mL*3). The combined organic layers were dried over Na₂SO₄, filtered, and concentrated under reduced pressure to give a residue. The residue was purified by flash silica gel chromatography (ISCO®; 4 g SepaFlash® Silica Flash Column, Eluent of 0˜65% Ethylacetate/MeOH @ 18 mL/min) and concentrated under reduced pressure to give a residue. I-226-6 (58 mg, 34.28 μmol, 61.51% yield, 84% purity) was obtained as a white solid. LCMS (Method D): R^{t}=0.407 min, [M+H]⁺=1421.4.

### Step 4: Synthesis of I-226

To a solution of I-226-6 (48 mg, 33.78 μmol, 1 eq) in DCM (5 mL) was added TFA (368.40 mg, 3.23 mmol, 240.00 μL, 95.65 eq). The mixture was stirred at 25° C. for 0.5 hr. The reaction mixture was filtered, and concentrated under reduced pressure to give a residue. The crude product was purified by reversed phase column (0.1% TFA condition) followed by lyophilization to give product. I-226 (25 mg, 18.73 μmol, 55.45% yield, 100% purity, TFA) was obtained as a white solid. LCMS (Method D): R^{t}=0.299 min, [M+H]⁺=1220.4. SFC Data: SFC: R^{t}=2.116 min. ¹H NMR (400 MHZ, METHANOL-d4) δ=8.32 (s, 1H), 7.79-7.71 (m, 1H), 7.65-7.54 (m, 2H), 7.39-7.33 (m, 4H), 7.31 (d, J=3.6 Hz, 1H), 7.29-7.23 (m, 1H), 7.19-7.07 (m, 2H), 6.84 (s, 1H), 5.05-4.97 (m, 2H), 4.62 (t, J=8.4 Hz, 3H), 4.54-4.46 (m, 1H), 4.34-4.24 (m, 5H), 4.14-3.96 (m, 4H), 3.94 (s, 3H), 3.80 (d, J=2.4 Hz, 5H), 3.74-3.57 (m, 4H), 3.55-3.41 (m, 3H), 3.27-3.17 (m, 3H), 3.15-3.01 (m, 2H), 2.64-2.49 (m, 2H), 2.38-2.24 (m, 2H), 2.19-1.92 (m, 7H), 1.91-1.74 (m, 6H), 1.69 (s, 3H), 1.61-1.51 (m, 1H), 1.38-1.05 (m, 6H). ¹⁹F NMR (400 MHZ, METHANOL-d4) δ=−76.956, −108.202, −112.912, −117.790.

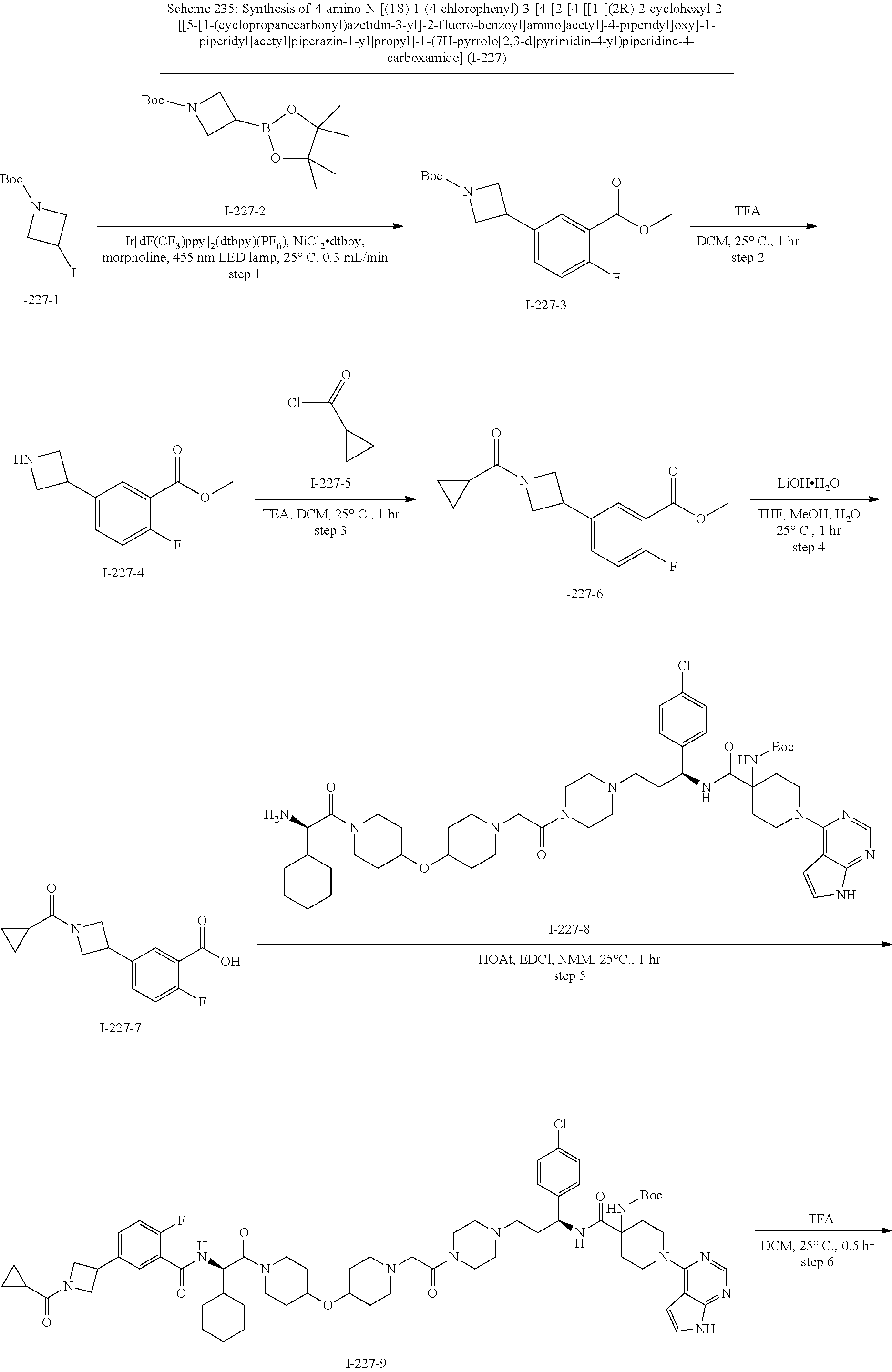

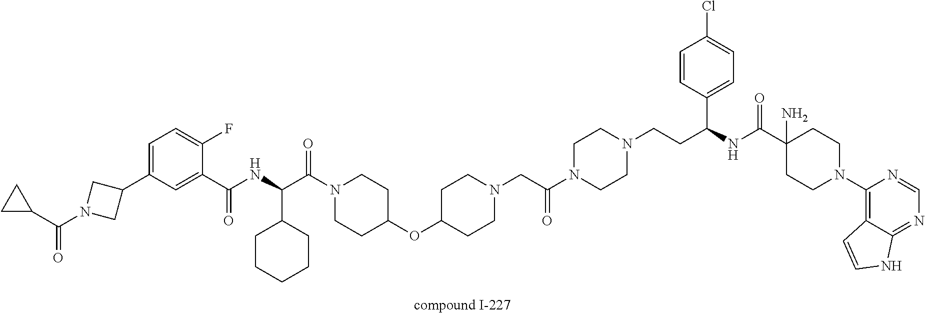 [SMILES: NC1(C(=O)N[C@@H](CCN2CCN(C(=O)CN3CCC(OC4CCN(C(=O)[C@H](NC(=O)c5cc(C6CN(C(=O)C7CC7)C6)ccc5F)C5CCCCC5)CC4)CC3)CC2)c2ccc(Cl)cc2)CCN(c2ncnc3[nH]ccc23)CC1]

### Step 1: Synthesis of I-227-3

To an 15 mL vial equipped with a stir bar was added methyl I-227-1 (1 g, 4.29 mmol, 1 eq), I-227-2 (1.22 g, 4.29 mmol, 1 eq) Ir[dF(CF3)ppy]2(dtbpy)(PF6) (48.14 mg, 42.91 μmol, 0.01 eq), NiCl2·tbpy (85.39 mg, 214.56 μmol, 0.05 eq), morpholine (560.78 mg, 6.44 mmol, 566.44 μL, 1.5 eq) in DMF (10 mL). The reaction solution was pumped through the reactor at a flow rate of 0.3 ml/min, irradiating with a 455 nm LED lamp with the flow wizard program, with cooling water to keep the reaction temperature at 25° C. for 1 hr. The reaction mixture was extracted with DCM (30 mL*3). The combined organic layers were dried over Na₂SO₄, filtered, and concentrated under reduced pressure to give a residue. The residue was purified by flash silica gel chromatography (ISCO®; 40 g SepaFlash® Silica Flash Column, Eluent of 0˜37% Ethylacetate/Petroleum ether gradient @ 60 mL/min) and concentrated under reduced pressure to give a residue. I-227-3 (1.14 g, 3.61 mmol, 84.16% yield, 98% purity) was obtained as a colorless oil. LCMS (Method D): R^{t}=0.425 min, [M+H]⁺=332.0. ¹H NMR (400 MHZ, METHANOL-d4) δ=7.87-7.86 (m, 1H), 7.60-7.58 (m, 1H), 7.22-7.18 (m, 1H), 4.35 (t, J=8.4 Hz, 2H), 4.03-3.96 (m, 1H), 3.91 (s, 3H), 3.88-3.81 (m, 2H), 1.47 (s, 9H). ¹⁹F NMR (377 MHz, METHANOL-d4), δ=−114.240 (s).

### Step 2: Synthesis of I-227-4

To a solution of I-227-3 (1.14 g, 3.69 mmol, 1 eq) in DCM (11 mL) was added TFA (8.75 g, 76.73 mmol, 5.70 mL, 20.82 eq). The mixture was stirred at 25° C. for 1 hr. The reaction mixture was filtered, and concentrated under reduced pressure to give product. I-227-4 (1.75 g, crude) was obtained as a red oil. LCMS (Method D): R^{t}=0.141 min, [M+H]⁺=210.2.

### Step 3: Synthesis of I-227-6

To a solution of I-227-4 (400 mg, 1.91 mmol, 1 eq) in DCM (4 mL) was added TEA (386.93 mg, 3.82 mmol, 532.22 μL, 2 eq), the mixture was added I-227-5 (219.85 mg, 2.10 mmol, 190.84 μL, 1.1 eq) at 0° C. Then mixture was stirred at 25° C. for 1 hr. The reaction mixture was extracted with DCM (10 ml*3). The combined organic layers were dried over Na₂SO₄, filtered, and concentrated under reduced pressure to give a residue. The crude product was purified by reversed phase column (0.1% FA condition) followed by lyophilization to give product. I-227-6 (280 mg, 868.40 μmol, 45.42% yield, 86% purity) was obtained as a yellow oil. LCMS (Method D): R^{t}=0.340 min, [M+H]⁺=278.2.

### Step 4: Synthesis of I-227-7

To a solution of I-227-6 (255 mg, 919.61 μmol, 1 eq) in THF (2.5 mL), MeOH (2.5 mL) and H₂O (1.25 mL) was added LiOH·H₂O (192.95 mg, 4.60 mmol, 5 eq). The mixture was stirred at 25° C. for 1 hr. The reaction mixture was concentrated under reduced pressure and adjusted pH=4 with HCl (1 M). Then the mixture was extracted with DCM (15 mL*3). The combined organic layers were dried over Na₂SO₄, filtered, and concentrated under reduced pressure to give a residue. The crude product was used into the next step without further purification. I-227-7 (145 mg, 550.78 μmol, 59.89% yield) was obtained as a colorless oil. LCMS (Method D): R^{t}=0.293 min, [M+H]⁺=264.0.

### Step 5: Synthesis of I-227-9

To a solution of I-227-7 (21 mg, 79.77 μmol, 1.5 eq) and I-227-8 (51.09 mg, 53.18 μmol, 1 eq) in DMF (0.2 mL) was added EDCI (30.58 mg, 159.54 μmol, 3 eq), NMM (26.89 mg, 265.89 μmol, 29.23 μL, 5 eq) and HOAt (7.24 mg, 53.18 μmol, 7.44 μL, 1 eq). The mixture was stirred at 25° C. for 1 hr. The reaction mixture was extracted with DCM (5 mL*3). The combined organic layers were dried over Na₂SO₄, filtered, and concentrated under reduced pressure to give a residue. The residue was purified by flash silica gel chromatography (ISCO®; 4 g SepaFlash® Silica Flash Column, Eluent of 0˜60% Ethylacetate/MeOH (@ 18 mL/min) and concentrated under reduced pressure to give a residue. I-227-9 (53 mg, 41.31 μmol, 77.69% yield, 94% purity) was obtained as a white solid. LCMS (Method D): R^{t}=0.363 min, [M+H]⁺=603.8.

### Step 6: Synthesis of I-227

To a solution of I-227-9 (43 mg, 35.66 μmol, 1 eq) in DCM (5 mL) was added TFA (330.03 mg, 2.89 mmol, 215.00 μL, 81.17 eq). The mixture was stirred at 25° C. for 0.5 hr. The reaction mixture was filtered, and concentrated under reduced pressure to give a residue. The crude product was purified by reverse-phase (0.1% TFA condition) followed by lyophilization to give product. I-227 (22.73 mg, 18.46 μmol, 51.77% yield, 99.058% purity, TFA) was obtained as a white solid. LCMS (Method D): R^{t}=0.322 min, [M+H]⁺=1105.4. ¹H NMR (400 MHZ, METHANOL-d4) δ=8.36 (s, 1H), 7.75 (t, J=6.4 Hz, 1H), 7.61-7.53 (m, 1H), 7.41-7.32 (m, 5H), 7.28-7.21 (m, 1H), 6.89 (d, J=2.0 Hz, 1H), 5.06-5.00 (m, 1H), 4.97 (d, J=7.6 Hz, 1H), 4.76 (t, J=8.4 Hz, 1H), 4.66-4.58 (m, 2H), 4.44-4.28 (m, 4H), 4.12-3.90 (m, 5H), 3.90-3.76 (m, 5H), 3.72 (s, 2H), 3.67-3.52 (m, 2H), 3.51-3.42 (m, 2H), 3.41-3.34 (m, 3H), 3.29-3.22 (m, 2H), 3.21-3.06 (m, 2H), 2.69-2.53 (m, 2H), 2.42-2.26 (m, 2H), 2.22-1.92 (m, 7H), 1.91-1.74 (m, 6H), 1.74-1.65 (m, 3H), 1.65-1.51 (m, 2H), 1.38-1.20 (m, 3H), 1.20-1.10 (m, 2H), 0.93-0.80 (m, 4H). ¹⁹F NMR (377 MHz, METHANOL-d4), δ=−76.964 (s), −118.181 (s).

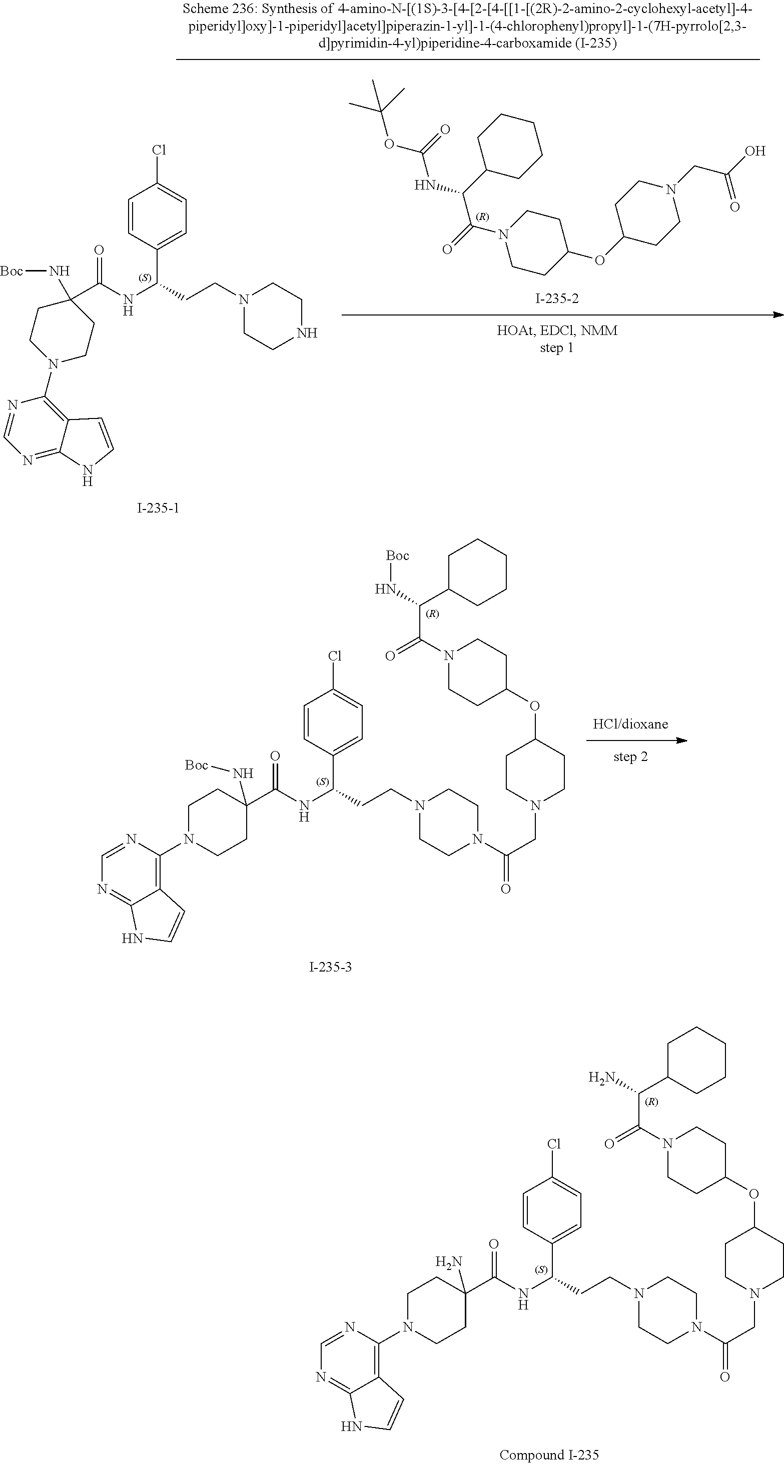

### Step 1: Synthesis of I-235-3

To a solution of I-235-1 (160 mg, 267.94 μmol, 1 eq), EDCI (102.73 mg, 535.88 μmol, 2 eq), HOAt (36.47 mg, 267.94 μmol, 37.48 μL, 1 eq) and NMM (135.51 mg, 1.34 mmol, 147.29 μL, 5 eq) in DMF (2 mL) was added I-235-2 (129.05 mg, 267.94 μmol, 1 eq). The mixture was stirred at 25° C. for 2 hr. The mixture was diluted with water (2 mL), extracted with EtOAc (2 mL×3). The organic layer was concentrated under vacuum. The residue was purified by Prep-HPLC (column: Waters Xbridge 150*25 mm*5 um; mobile phase: [water (ammonia hydroxide v/v)-ACN]; gradient: 45%-75% B over 12 min) and dried by lyophilization. I-235-3 (150 mg, 141.41 μmol, 52.78% yield) as yellow oil. LCMS: R^{t}=0.767 min, [M+H]⁺=1060.7.

### Step 2: Synthesis of I-235

To a solution of I-235-3 (150 mg, 141.41 μmol, 1 eq) in DCM (1 mL) was added HCl/dioxane (2 M, 4 mL, 56.57 eq). The mixture was stirred at 25° C. for 1 hr. The reaction mixture was concentrated under vacuum. The residue was dried by lyophilization without purification. I-235 (78.87 mg, 85.86 μmol, 60.72% yield, 97.65% purity, HCl) as an off-white solid. LCMS: R^{t}=0.360 min, [M+H]⁺=860.7. SFC: R^{t}=2.296 min, ee %=96.378%. ¹H NMR (400 MHz, METHANOL-da) δ=8.39 (s, 1H), 7.47 (br d, J=8.0 Hz, 2H), 7.42 (br d, J=4.0 Hz, 1H), 7.32 (br d, J=8.0 Hz, 2H), 7.02 (br s, 1H), 5.10 (br d, J=4.0 Hz, 1H), 4.54 (br s, 4H), 4.37 (br s, 2H), 4.26-4.08 (m, 3H), 4.02 (br s, 1H), 3.96 (br s, 1H), 3.90-3.79 (m, 3H), 3.65 (s, 6H), 3.50 (br d, J=12.0 Hz, 3H), 3.45-3.36 (m, 4H), 2.89-2.74 (m, 2H), 2.57 (br d, J=8.0 Hz, 1H), 2.38 (br d, J=12.0 Hz, 2H), 2.20 (br d, J=12.0 Hz, 2H), 2.08 (br s, 2H), 1.82 (br d, J=12.0 Hz, 6H), 1.76-1.63 (m, 4H), 1.33-1.15 (m, 6H).

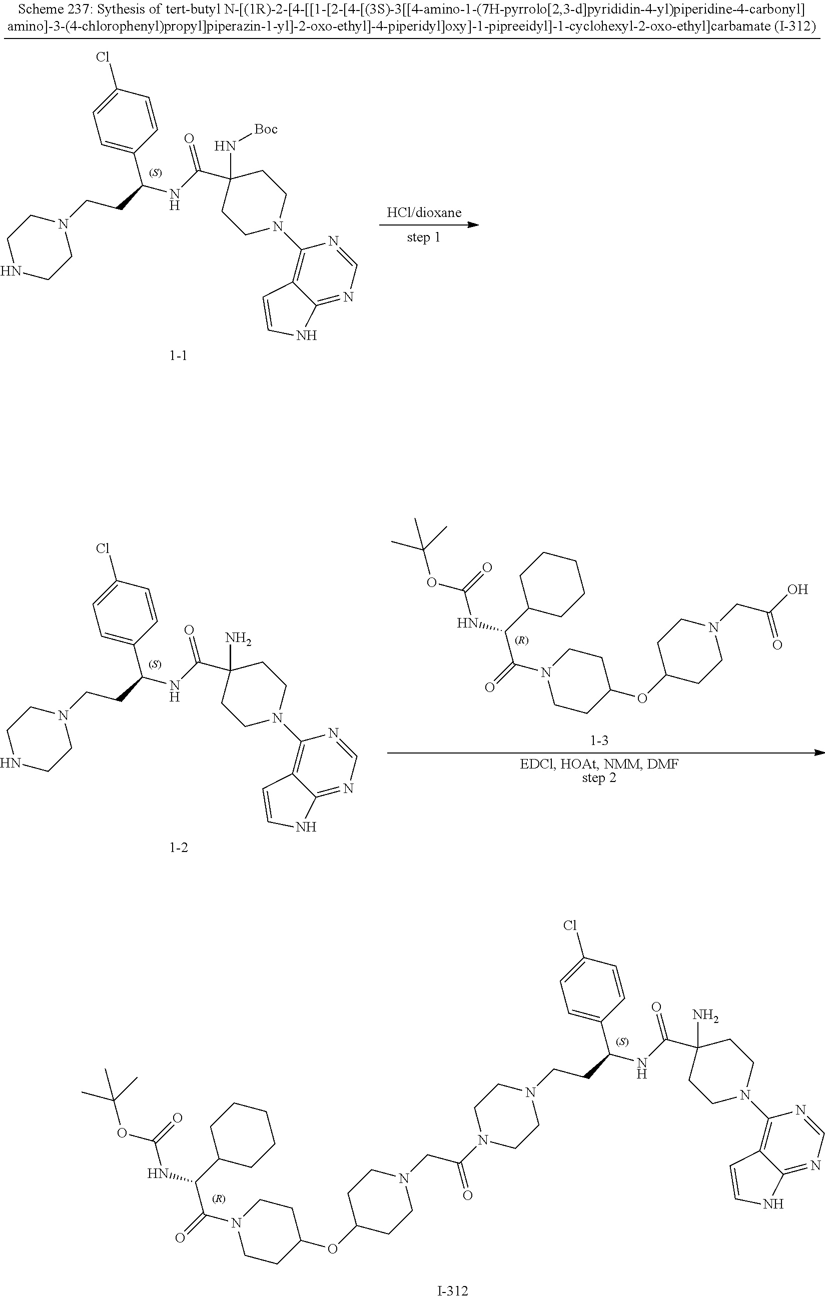

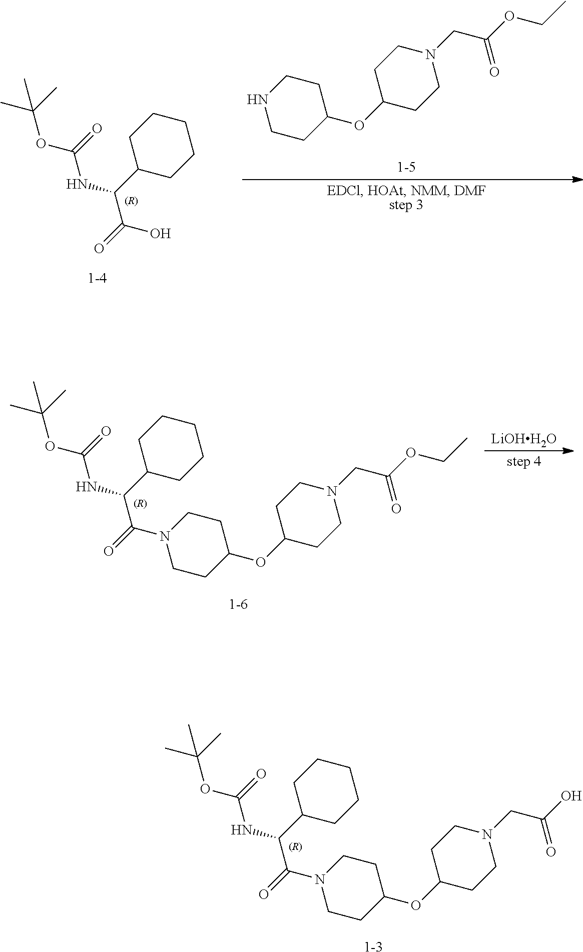

### Step 1: Synthesis of 1-2

To a solution of 1-1 (200 mg, 334.92 μmol, 1 eq) in DCM (2 mL) was added HCl/dioxane (2 M, 4.00 mL, 23.89 eq). The mixture was stirred at 25° C. for 1 hr. The reaction mixture was concentrated under vacuum. This reaction was used in next step without purification. 1-2 (160 mg, crude, HCl) as a white solid was obtained. LCMS: R^{t}=0.351 min, [M+H]⁺=497.4.

### Step 2: Synthesis of I-312

To a solution of 1-2 (160 mg, 299.91 μmol, 1 eq, HCl), EDCI (114.99 mg, 599.82 μmol, 2 eq), HOAt (40.82 mg, 299.91 μmol, 41.95 μL, 1 eq) and NMM (151.67 mg, 1.50 mmol, 164.86 μL, 5 eq) in DMF (2 mL) was added 1-3 (144.44 mg, 299.91 μmol, 1 eq). The mixture was stirred at 25° C. for 1.5 hr. The mixture was diluted with water (2 mL), extracted with EtOAc (2 mL×3). The organic layer was concentrated under vacuum. The residue was purified by Prep-HPLC (column: Waters Xbridge 150*25 mm*5 um; mobile phase: [water (ammonia hydroxide v/v)-ACN]; gradient: 36%-66% B over 12 min) and dried by lyophilization. I-312 (37.29 mg, 38.00 μmol, 12.67% yield, 97.90% purity) as a white solid was obtained. LCMS: R^{t}=0.700 min, [M+H]⁺=961.6. SFC: R^{t}=0.918 min, ee %=100%. ¹H NMR (400 MHz, METHANOL-d4) δ=8.12 (s, 1H), 7.32 (s, 4H), 7.12 (d, J=4.0 Hz, 1H), 6.61 (d, J=4.0 Hz, 1H), 4.99-4.95 (m, 1H), 4.54-4.45 (m, 2H), 4.37 (br d, J=8.0 Hz, 1H), 4.03-3.89 (m, 1H), 3.86-3.80 (m, 1H), 3.76-3.71 (m, 1H), 3.69-3.60 (m, 6H), 3.53-3.48 (m, 1H), 3.42-3.35 (m, 1H), 3.17 (s, 2H), 2.79-2.71 (m, 2H), 2.52-2.47 (m, 1H), 2.46-2.41 (m, 2H), 2.41-2.37 (m, 1H), 2.36-2.32 (m, 2H), 2.29-2.23 (m, 2H), 2.22-2.13 (m, 2H), 2.08-2.02 (m, 1H), 2.00-1.94 (m, 1H), 1.92-1.82 (m, 4H), 1.75 (br d, J=8.0 Hz, 4H), 1.68-1.55 (m, 8H), 1.54 (br d, J=4.0 Hz, 1H), 1.43 (s, 9H), 1.28-1.18 (m, 3H), 1.09-0.98 (m, 2H).

### Step 3: Synthesis of 1-6

To a solution of 1-4 (2.5 g, 9.72 mmol, 1 eq) in DMF (25 mL) was added 1-5 (2.98 g, 9.72 mmol, 1 eq, HCl), HOAt (1.32 g, 9.72 mmol, 1.36 mL, 1 eq), EDCI (3.72 g, 19.43 mmol, 2 eq) and NMM (4.91 g, 48.58 mmol, 5.34 mL, 5 eq). The mixture was stirred at 25° C. for 0.5 h. The reaction mixture was quenched by H₂O (40 mL), extracted with EA (30 mL*3). The combined organic layers were washed with brine (30 mL*3), dried over anhydrous Na₂SO₄, filtered, and concentrated under reduced pressure to give a residue. The residue was purified by reverse-phase HPLC (0.1% NH₃·H₂O), 1-6 (4 g, 7.58 mmol, 77.99% yield, 96.549% purity) was obtained as yellow oil. LCMS: R^{t}=0.653 min, [M+H]⁺=510.4.

### Step 4: Synthesis of 1-3

To a solution of 1-6 (260 mg, 510.13 μmol, 1 eq) in MeOH (1 mL), THF (1 mL) and H₂O (1 mL) was added LiOH·H₂O (42.81 mg, 1.02 mmol, 2 eq). The mixture was stirred at 25° C. for 2 hr. The mixture was concentrated under vacuum to except MeOH. The mixture was adjusted to pH=6 with aq. HCl and extracted with EA (2 ml*3) as well as Cl₃CH/IPA (2 ml*3, v/v=5:1). The organic layer was dried with anhydrous Na₂SO₄, filtered, and concentrated under vacuum. This residue was used in next step without purification 1-3 (290 mg, crude) was obtained as light yellow oil. LCMS: R^{t}=0.456 min, [M+H]⁺=482.4. ¹H NMR (400 MHZ, METHANOL-d4) δ=5.49 (s, 3H), 4.41-4.36 (m, 1H), 4.01-3.90 (m, 1H), 3.85 (br s, 1H), 3.82-3.76 (m, 2H), 3.60 (d, J=4.0 Hz, 2H), 3.56-3.49 (m, 1H), 3.45-3.38 (m, 2H), 3.29-3.21 (m, 1H), 2.07 (br s, 2H), 1.99 (s, 1H), 1.96-1.91 (m, 3H), 1.85-1.80 (m, 1H), 1.76 (br d, J=8.0 Hz, 3H), 1.70-1.56 (m, 5H), 1.55-1.49 (m, 1H), 1.43 (d, J=4.0 Hz, 9H), 1.09-0.99 (m, 2H).

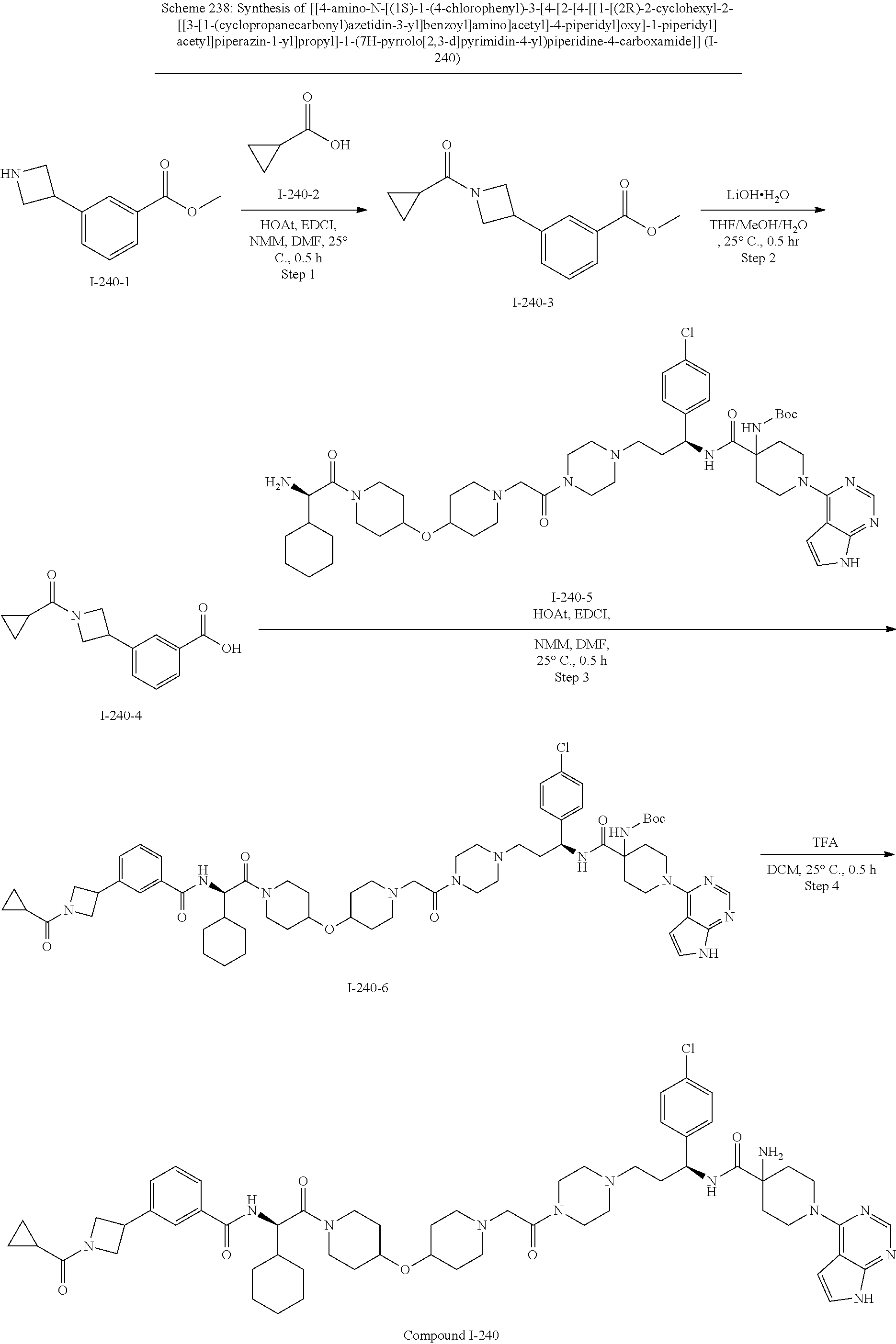

### Step 1: Synthesis of I-240-3

To a solution of methyl I-240-1 (104.82 mg, 343.38 μmol, 1 eq, TFA) and I-240-2 (35.47 mg, 412.06 μmol, 32.60 μL, 1.2 eq) in DMF (1 mL) was added EDCI (197.48 mg, 1.03 mmol, 3 eq), HOAt (46.74 mg, 343.38 μmol, 48.03 μL, 1 eq) and NMM (173.66 mg, 1.72 mmol, 188.76 μL, 5 eq). Then the mixture was stirred at 25° C. for 0.5 hr. The reaction mixture was poured into H₂O (2 mL), extract with EA (2 mL*3). The combined organic layers were dried over Na₂SO₄, filtered, and concentrated under reduced pressure to give a residue. The residue was purified by flash silica gel chromatography (ISCO®; 12 g SepaFlash® Silica Flash Column, Eluent of 0˜100% PE/EA @, 36 mL/min, PE/EA=1:1, Rf=0.3) and the eluent was concentrated under reduced pressure to give a product. I-240-3 (130 mg, crude) was obtained as a white gum. LCMS (Method D): R^{t}=0.356 min, [M+H]′=260.1.

### Step 2: Synthesis of I-240-4

To a solution of I-240-3 (70 mg, 269.96 μmol, 1 eq) in THF (0.2 mL), MeOH (0.2 mL) and H₂O (0.2 mL) was added LiOH·H₂O (33.99 mg, 809.87 μmol, 3 eq). The mixture was stirred at 25° C. for 0.5 hr. The reaction mixture was poured into H₂O (10 mL) at 25° C., and then adjusted to pH=7 with 1M aqueous HCl, and then extracted with DCM 2 mL*3. The combined organic layers were dried over Na₂SO₄, filtered, and concentrated under reduced pressure to give product. I-240-4 (50 mg, 203.85 μmol, 75.51% yield, 100% purity) was obtained as a white gum. LCMS (Method D): R^{t}=0.306 min, [M+H]′=246.1. 1H NMR (400 MHZ, CHLOROFORM-d) δ=8.14 (s, 1H), 7.99 (d, J=7.6 Hz, 1H), 7.53-7.36 (m, 2H), 4.69 (t, J=7.6 Hz, 1H), 4.44 (t, J=8.8 Hz, 1H), 4.30 (s, 1H), 4.18 (d, J=6.2 Hz, 1H), 3.96-3.78 (m, 1H), 1.45 (d, J=4.0 Hz, 1H), 1.04 (s, 2H), 0.8-0.78 (m, 2H).

### Step 3: Synthesis of I-240-6

To a solution of I-240-5 (50 mg, 52.05 μmol, 1 eq) and I-240-4 (19.15 mg, 78.07 μmol, 1.5 eq) in DMF (0.5 mL) was added EDCI (29.93 mg, 156.15 μmol, 3 eq), HOAt (7.08 mg, 52.05 μmol, 7.28 μL, 1 eq) and NMM (26.32 mg, 260.24 μmol, 28.61 μL, 5 eq). The mixture was stirred at 25° C. for 0.5 hr. The reaction mixture was poured into H₂O (2 mL), extracted with EA (2 mL*3). The combined organic layers were dried over Na₂SO₄, filtered, and concentrated under reduced pressure to give a residue. The residue was purified by flash silica gel chromatography (ISCO®; 4 g SepaFlash® Silica Flash Column, Eluent of 0˜100% DCM/MeOH @ 18 mL/min, DCM/MeOH=10:1, Rf=0.4) and the eluent was concentrated under reduced pressure to give a product. I-240-6 (25 mg, 21.05 μmol, 40.43% yield) was obtained as a yellow gum. LCMS (Method D): R^{t}=0.349 min, [M+H]′=1188.9.

### Step 4: Synthesis of I-240

To a solution of I-240-6 (25 mg, 21.05 μmol, 1 eq) in DCM (0.25 mL) was added TFA (76.75 mg, 673.11 μmol, 0.05 mL, 31.98 eq). The mixture was stirred at 25° C. for 0.5 hr. The reaction mixture was concentrated under reduced pressure to give a residue. The residue was purified by prep-HPLC (column: CD01-Phenomenex luna C18 150*25*10 um; mobile phase: [water (TFA)-ACN]; gradient: 16%-46% B over 10 min) and the eluent was concentrated to remove MeCN and then lyophilization to give product. I-240 (6.31 mg, 5.24 μmol, 24.89% yield, 99.77% purity, TFA) was obtained as a white solid. LCMS (Method D): R^{t}=0.306 min, [M+H]⁺=1087.6. SFC: R^{t}=. ¹H NMR (400 MHz, METHANOL-d4) δ=8.50-8.41 (m, 1H), 8.37 (s, 1H), 7.87 (s, 1H), 7.75 (d, J=7.6 Hz, 1H), 7.59-7.54 (m, 1H), 7.52-7.46 (m, 1H), 7.36 (s, 5H), 6.91 (s, 1H), 5.06-5.00 (m, 2H), 4.96-4.92 (m, 4H), 4.80-4.76 (m, 1H), 4.68-4.61 (m, 2H), 4.45-4.38 (m, 2H), 4.30 (d, J=9.6 Hz, 2H), 4.10-3.97 (m, 4H), 3.94 (br s, 1H), 3.82-3.78 (m, 5H), 3.74-3.65 (m, 3H), 3.51-3.40 (m, 4H), 3.22-3.03 (m, 3H), 2.68-2.54 (m, 2H), 2.39-2.26 (m, 2H), 2.22-2.11 (m, 2H), 2.04 (s, 4H), 1.98-1.84 (m, 5H), 1.79 (d, J=9.6 Hz, 2H), 1.72-1.63 (m, 4H), 1.34-1.23 (m, 3H), 1.17-1.08 (m, 2H), 0.91-0.82 (m, 4H). F NMR (400 MHZ, METHANOL-d4) δ=−77.061.

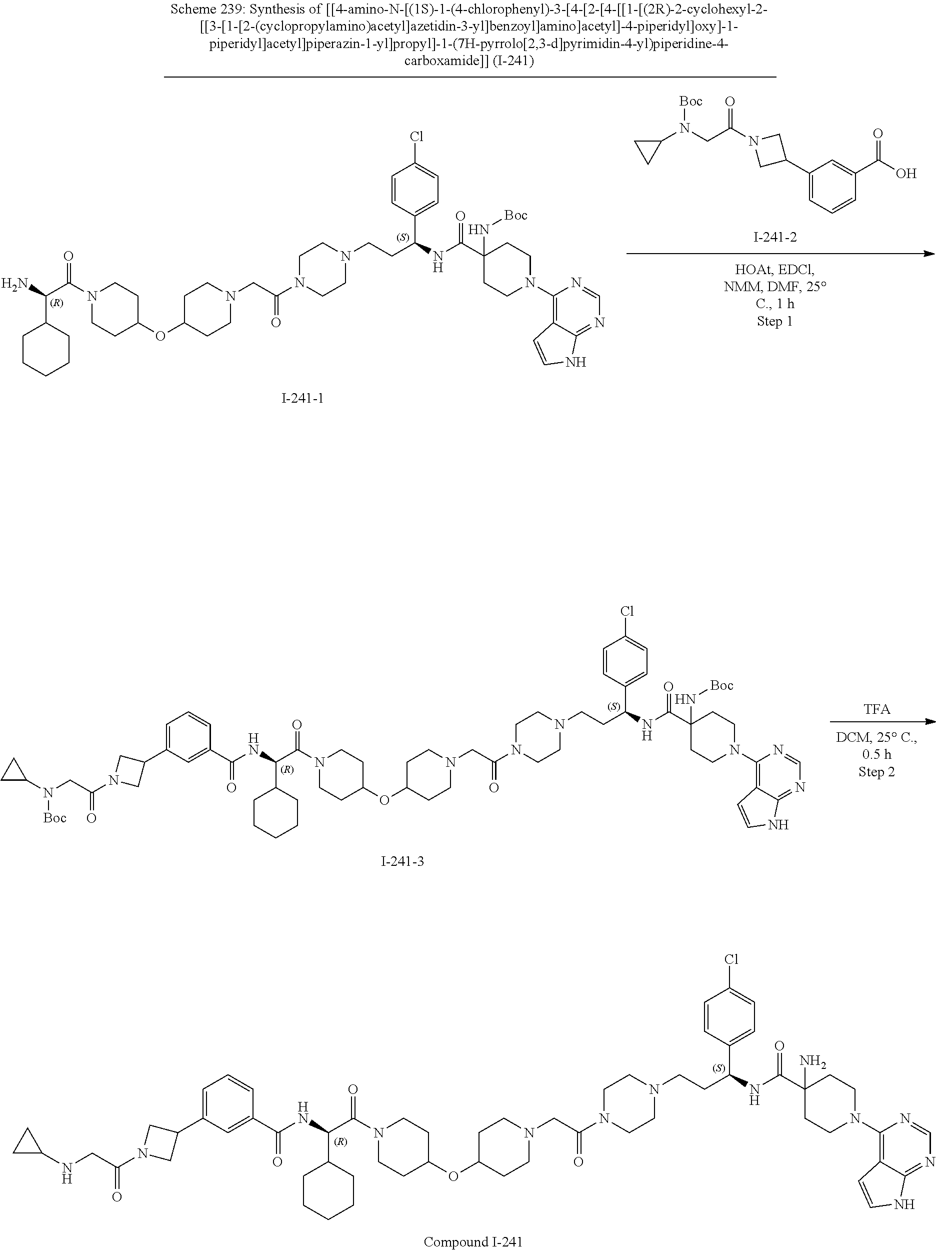

### Step 1: Synthesis of I-241-3

To a solution of I-241-1 (50 mg, 52.05 μmol, 1 eq) and I-241-2 (23.39 mg, 62.46 μmol, 1.2 eq) in DMF (0.5 mL) was added EDCI (29.93 mg, 156.15 μmol, 3 eq), HOAt (7.08 mg, 52.05 μmol, 7.28 μL, 1 eq) and NMM (26.32 mg, 260.24 μmol, 28.61 μL, 5 eq). The mixture was stirred at 25° C. for 1 hr. The reaction mixture was poured into H₂O (2 mL), extracted with EA (2 mL*3). The combined organic layers were dried over Na₂SO₄, filtered, and concentrated under reduced pressure to give a residue. The residue was purified by flash silica gel chromatography (ISCO®; 4 g SepaFlash® Silica Flash Column, Eluent of 0˜100% DCM/MeOH @ 18 mL/min, DCM/MeOH=10:1, Rf=0.3) and the eluent was concentrated under reduced pressure to give a product. I-241-3 (60 mg, 45.56 μmol, 87.53% yield) was obtained as a yellow gum. LCMS (Method D): R^{t}=0.388 min, [M+H]⁺=1316.3.

### Step 2: Synthesis of I-241

To a solution of I-241-3 (60 mg, 45.56 μmol, 1 eq) in DCM (0.6 mL) was added TFA (184.20 mg, 1.62 mmol, 0.12 mL, 35.46 eq). The mixture was stirred at 25° C. for 0.5 hr. The reaction mixture was concentrated under reduced pressure to give a residue. The residue was purified by prep-HPLC (column: CD02-Waters Xbidge BEH C18 150*25*10 um; mobile phase: [water (TFA)-ACN]; gradient: 10%-40% B over 10 min) and the eluent was concentrated to remove MeCN and then lyophilization to give product. I-241 (13.43 mg, 10.70 μmol, 23.48% yield, 98.05% purity, TFA) was obtained as a white solid. LCMS (Method D): R^{t}=0.272 min, [M+H]⁺=1116.4. SFC: R^{t}=0.894 min. ¹H NMR (400 MHZ, METHANOL-d4) δ=8.42-8.30 (m, 2H), 7.84 (d, J=5.6 Hz, 1H), 7.76 (d, J=8.0 Hz, 1H), 7.62-7.55 (m, 1H), 7.53-7.46 (m, 1H), 7.36 (s, 4H), 6.91 (s, 1H), 5.06-4.99 (m, 1H), 4.98-4.89 (m, 3H), 4.70-4.60 (m, 3H), 4.55-4.49 (m, 1H), 4.35-4.27 (m, 3H), 4.17-1.09 (m, 2H), 4.02 (s, 3H), 3.97-3.92 (m, 1H), 3.87-3.77 (m, 4H), 3.76-3.59 (m, 4H), 3.56-3.37 (m, 5H), 3.24-3.06 (m, 3H), 2.84-2.78 (m, 1H), 2.70-2.55 (m, 2H), 2.42-2.25 (m, 2H), 2.23-2.11 (m, 2H), 2.11-1.95 (m, 5H), 1.94-1.82 (m, 4H), 1.81-1.75 (m, 2H), 1.73-1.63 (m, 3H), 1.61-1.50 (m, 1H), 1.40-1.17 (m, 4H), 1.17-1.07 (m, 2H), 0.95-0.88 (m, 4H). ¹⁹F NMR (400 MHZ, METHANOL-d4) δ=−77.037.

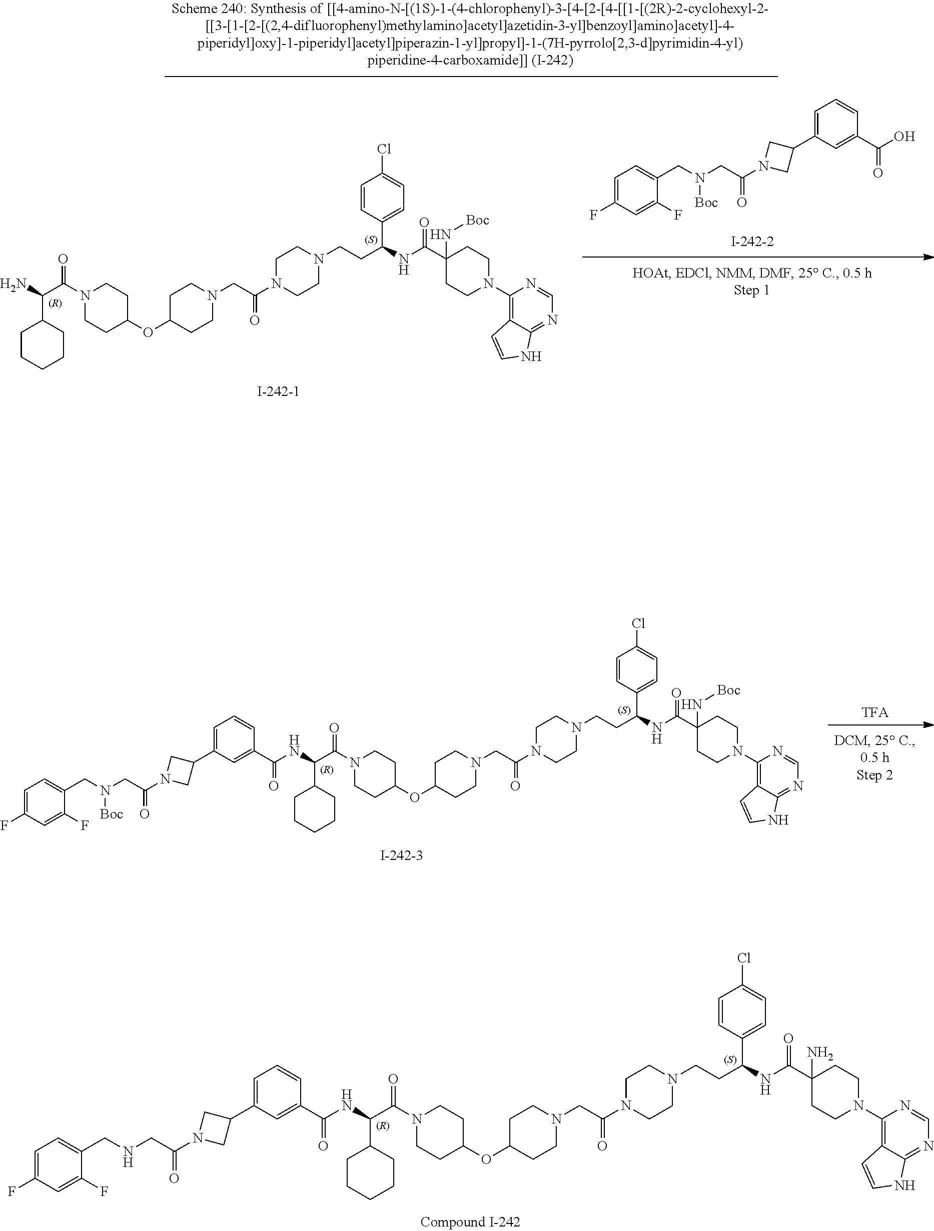

### Step 1: Synthesis of I-242-3

To a solution of I-242-1 (50 mg, 52.05 μmol, 1 eq) and I-242-2 (23.97 mg, 52.05 μmol, 1 eq) in DMF (0.5 mL) was added EDCI (29.93 mg, 156.15 μmol, 3 eq), HOAt (7.08 mg, 52.05 μmol, 7.28 μL, 1 eq) and NMM (26.32 mg, 260.24 μmol, 28.61 μL, 5 eq). The mixture was stirred at 25° C. for 0.5 hr. The reaction mixture was poured into H₂O (2 mL), extract with EA (2 mL*3). The combined organic layers were dried over Na₂SO₄, filtered, and concentrated under reduced pressure to give a residue. The residue was purified by flash silica gel chromatography (ISCO®; 4 g SepaFlash® Silica Flash Column, Eluent of 0˜100% DCM/MeOH @ 18 mL/min, DCM/MeOH=10:1, Rf=0.3) and the eluent was concentrated under reduced pressure to give a product. I-242-3 (60 mg, 42.76 μmol, 82.16% yield) was obtained as a yellow gum. LCMS (Method D): R^{t}=0.389 min, [M+H]⁺=1403.6.

### Step 2: Synthesis of I-242

To a solution of I-242-3 (55 mg, 39.20 μmol, 1 eq) in DCM (0.5 mL) was added TFA (153.50 mg, 1.35 mmol, 0.1 mL, 34.34 eq). The mixture was stirred at 25° C. for 0.5 hr. The reaction mixture was concentrated under reduced pressure to give a residue. The residue was purified by prep-HPLC (column: CD03-Welch Xtimate C18 150*25*5 um; mobile phase: [water (TFA)-ACN]; gradient: 10%-40% B over 10 min) and the eluent was concentrated to remove MeCN and then lyophilization to give product. I-242 (14.4 mg, 10.93 μmol, 27.90% yield, 100% purity, TFA) was obtained as a white solid. LCMS (Method D): R^{t}=0.292 min, [M+H]⁺=1202.3. SFC: R^{t}=1.017 min. ¹H NMR (400 MHz, METHANOL-d4) δ=8.46-8.23 (m, 2H), 7.84 (d, J=6.4 Hz, 1H), 7.77 (d, J=8.0 Hz, 1H), 7.65-7.54 (m, 2H), 7.53-7.47 (m, 1H), 7.40-7.32 (m, 5H), 7.20-7.06 (m, 2H), 6.94-6.82 (m, 1H), 5.03 (t, J=6.8 Hz, 1H), 4.98-4.89 (m, 2H), 4.68-4.58 (m, 3H), 4.55-4.49 (m, 1H), 4.41-4.20 (m, 6H), 4.19-3.98 (m, 4H), 3.95 (s, 3H), 3.88-3.75 (m, 5H), 3.74-3.58 (m, 4H), 3.57-3.38 (m, 4H), 3.21-3.04 (m, 3H), 2.69-2.53 (m, 2H), 2.41-2.26 (m, 2H), 2.21-2.09 (m, 2H), 2.09-1.93 (m, 5H), 1.93-1.82 (m, 4H), 1.81-1.75 (m, 2H), 1.72-1.64 (m, 3H), 1.59-1.50 (m, 1H), 1.33-1.21 (m, 3H), 1.17-1.07 (m, 2H). ¹⁹F NMR (400 MHZ, METHANOL-d4) δ=−77.052, −77.047, −108.230, −112.928.

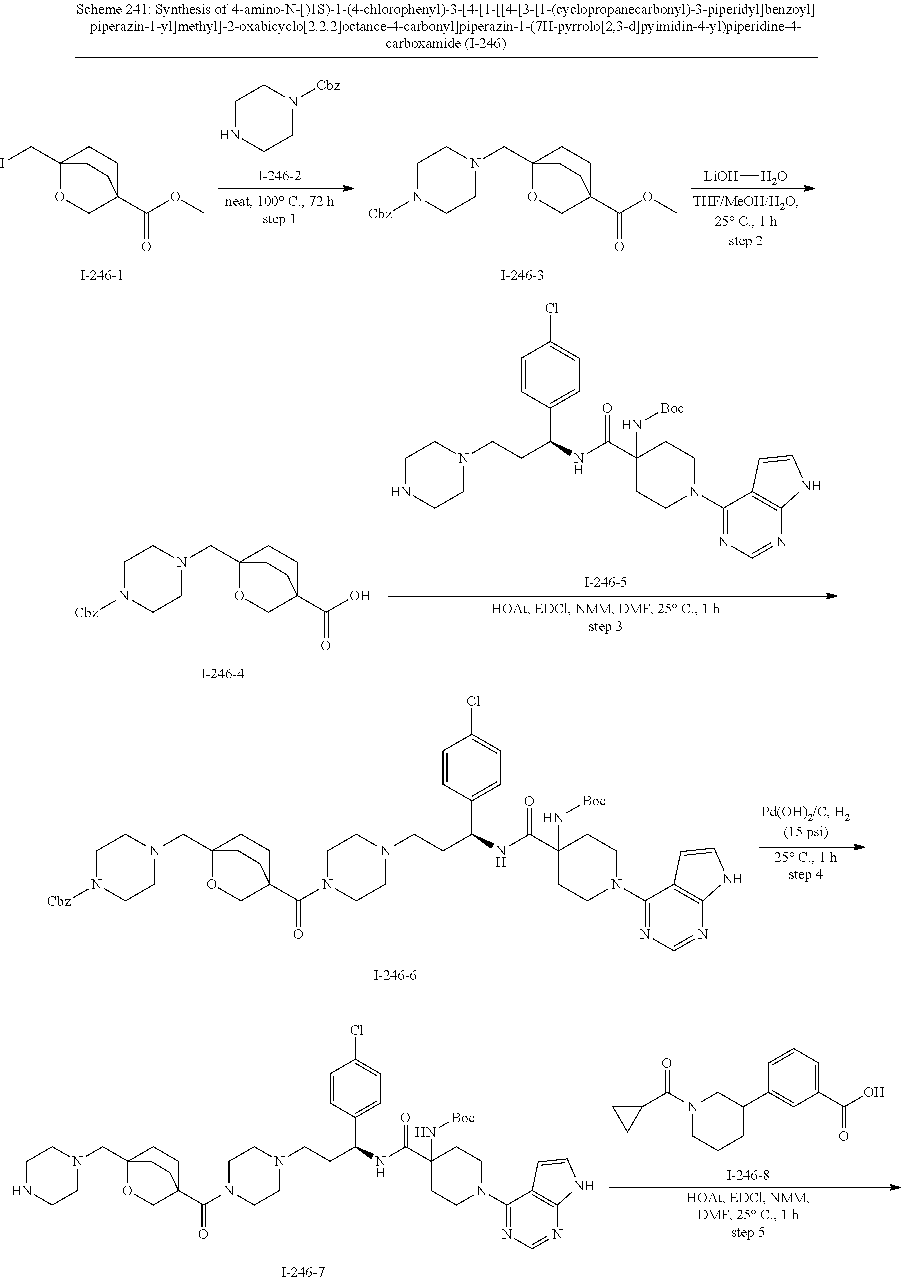

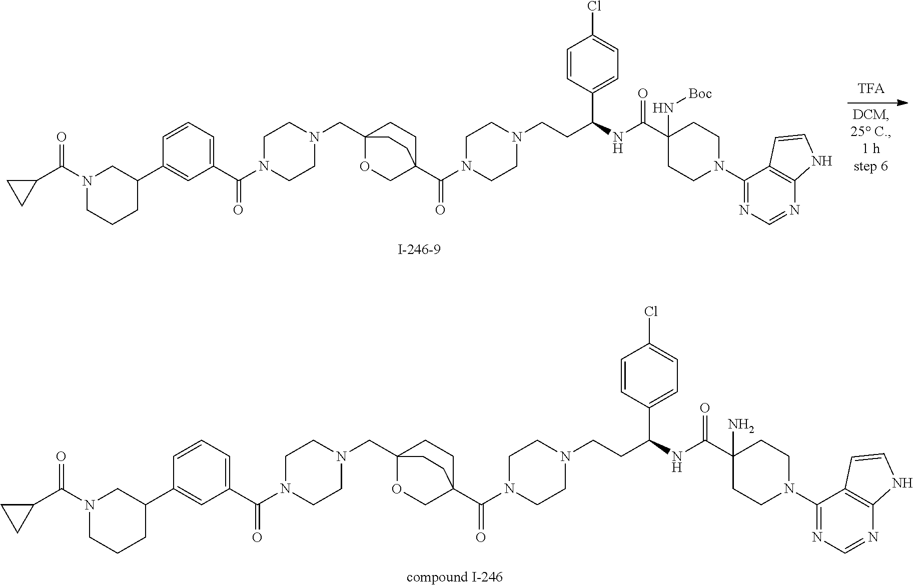 [SMILES: CC(C)(C)OC(=O)NC1(C(=O)N[C@@H](CCN2CCN(C(=O)C34CCC(CN5CCN(C(=O)c6cccc(C7CCCN(C(=O)C8CC8)C7)c6)CC5)(CC3)OC4)CC2)c2ccc(Cl)cc2)CCN(c2ncnc3[nH]ccc23)CC1 ; NC1(C(=O)N[C@@H](CCN2CCN(C(=O)C34CCC(CN5CCN(C(=O)c6cccc(C7CCCN(C(=O)C8CC8)C7)c6)CC5)(CC3)OC4)CC2)c2ccc(Cl)cc2)CCN(c2ncnc3[nH]ccc23)CC1]

### Step 1: Synthesis of I-246-3

A mixture of I-246-1 (9 g, 28.98 mmol, 1 eq) and I-246-2 (31.95 g, 145.08 mmol, 27.99 mL, 5 eq) was stirred at 100° C. for 72 hr. The mixture was purified by flash silica gel chromatography (ISCO®; 120 g SepaFlash® Silica Flash Column, Eluent of 0˜5% Ethyl acetate/Petroleum ether gradient @ 60 mL/min) and concentrated under reduced pressure to give a residue. I-246-3 (5.7 g, 14.16 mmol, 48.80% yield, 100% purity) was obtained as a white solid. LCMS (Method D): R^{t}=0.323 min, [M+H]⁺=403.6. ¹H NMR (400 MHz, METHANOL-d4) δ=7.41-7.24 (m, 5H), 5.10 (s, 2H), 3.89 (s, 2H), 3.64 (s, 3H), 3.46 (s, 4H), 2.53-2.44 (m, 4H), 2.26 (s, 2H), 1.97-1.90 (m, 6H), 1.68-1.55 (m, 2H).

### Step 2: Synthesis of I-246-4

To a solution of I-246-3 (5.2 g, 12.92 mmol, 1 eq) in THF (20 mL), MeOH (20 mL) and H₂O (10 mL) was added LiOH·H₂O (2.71 g, 64.60 mmol, 5 eq). The mixture was stirred at 25° C. for 1 hr. The mixture was concentrated under reduced pressure and adjusted to pH=7 with HCl (1 M). The mixture was extracted with solution (CHCl₃:IPA=3:1; 30 mL*3). The organic layers were dried over Na₂SO₄, filtered, and concentrated under reduced pressure to give a residue. I-246-4 (5.1 g, 12.08 mmol, 93.49% yield, 92% purity) was obtained as a light yellow solid. LCMS (Method D): R^{t}=0.229 min, [M+H]⁺=389.1. ¹H NMR (400 MHZ, METHANOL-d4) δ=7.39-7.26 (m, 5H), 5.11 (s, 2H), 3.90 (s, 2H), 3.53-3.42 (m, 4H), 2.57-2.47 (m, 4H), 2.29 (s, 2H), 1.96-1.88 (m, 6H), 1.66-1.54 (m, 2H).

### Step 3: Synthesis of I-246-6

To a solution of I-246-4 (350 mg, 901.00 μmol, 1 eq) and I-246-5 (538.03 mg, 901.00 μmol, 1 eq) in DMF (5 mL) was added EDCI (518.17 mg, 2.70 mmol, 3 eq), HOAT (122.64 mg, 901.00 μmol, 126.04 μL, 1 eq) and NMM (455.67 mg, 4.51 mmol, 495.29 μL, 5 eq). The mixture was stirred at 25° C. for 1 hr. The reaction mixture was concentrated under reduced pressure to give a residue. The residue was purified by flash silica gel chromatography (ISCO®; 20 g SepaFlash® Silica Flash Column, Eluent of 0˜60% Ethyl acetate/Methanol gradient (50 mL/min) and concentrated under reduced pressure to give a residue. I-246-6 (930 mg, 893.87 μmol, 99.21% yield, 93% purity) was obtained as a yellow oil. LCMS (Method D): R^{t}=0.323 min, [M+H]⁺=967.3. SFC: R^{t}=1.886 min.

### Step 4: Synthesis of I-246-7

A mixture of I-246-6 (760 mg, 785.46 μmol, 1 eq) in EtOH (8 mL) was added Pd(OH)₂/C (350.00 mg, 498.45 μmol, 20% purity; 6.35e-1 eq), degassed and purged with H₂ for 3 times, and then the mixture was stirred at 25° C. for 1 hr under H₂ (15 Psi). The reaction mixture was filtered, and concentrated under reduced pressure to give a residue. The crude product was purified by reversed phase column (Neutral condition) and lyophilization to give a product. I-246-7 (250 mg, 284.96 μmol, 36.28% yield, 95% purity) was obtained as a light yellow solid. LCMS (Method D): R^{t}=0.265 min, [M+H]⁺=833.9. SFC: R^{t}=1.810 min.

### Step 5: Synthesis of I-246-9

To a solution of I-246-7 (30 mg, 35.99 μmol, 1 eq) and I-246-8 (9.84 mg, 35.99 μmol, 1 eq) in DMF (0.3 mL) was added EDCI (20.70 mg, 107.98 μmol, 3 eq), HOAT (4.90 mg, 35.99 μmol, 5.04 μL, 1 eq) and NMM (18.20 mg, 179.97 μmol, 19.79 μL, 5 eq). The mixture was stirred at 25° C. for 1 hr. The reaction mixture was concentrated under reduced pressure to give a residue. The residue was purified by flash silica gel chromatography (ISCO®; 4 g SepaFlash® Silica Flash Column, Eluent of 0˜60% Ethyl acetate/Petroleum ether gradient @ 30 mL/min) and concentrated under reduced pressure to give a residue. I-246-9 (36 mg, 29.76 μmol, 82.67% yield, 90% purity) was obtained as a light yellow solid. LCMS: R^{t}=0.322 min, [M+H]⁺=1088.7.

### Step 6: Synthesis of I-246

To a solution of I-246-9 (36 mg, 33.06 μmol, 1 eq) in DCM (1 mL) was added TFA (0.4 mL). The mixture was stirred at 25° C. for 1 hr. The reaction mixture was concentrated under reduced pressure to give a residue. The crude product was purified by reversed phase column (Neutral condition) and lyophilized to give a residue. I-246 (4.09 mg, 4.10 μmol, 12.39% yield, 99.034% purity) was obtained as a white solid. LCMS: R^{t}=0.279 min, [M+H]⁺=988.3. SFC: R^{t}=2.023 min. ¹H NMR (400 MHZ, METHANOL-d4) 8=8.12 (s, 1H), 7.47-7.37 (m, 2H), 7.37-7.29 (m, 5H), 7.27 (t, J=6.4 Hz, 1H), 7.12 (d, J=3.6 Hz, 1H), 6.62 (d, J=3.6 Hz, 1H), 4.98 (t, J=6.4 Hz, 1H), 4.57-4.46 (m, 3H), 4.42-4.31 (m, 1H), 3.92 (s, 2H), 3.78-3.58 (m, 8H), 3.46-3.36 (m, 2H), 3.27-3.20 (m, 1H), 2.87-2.68 (m, 2H), 2.67-2.57 (m, 2H), 2.55-2.43 (m, 4H), 2.42-2.29 (m, 6H), 2.24-2.12 (m, 4H), 2.09-2.02 (m, 2H), 2.02-1.93 (m, 4H), 1.91-1.78 (m, 4H), 1.70-1.53 (m, 5H), 0.91-0.74 (m, 4H).

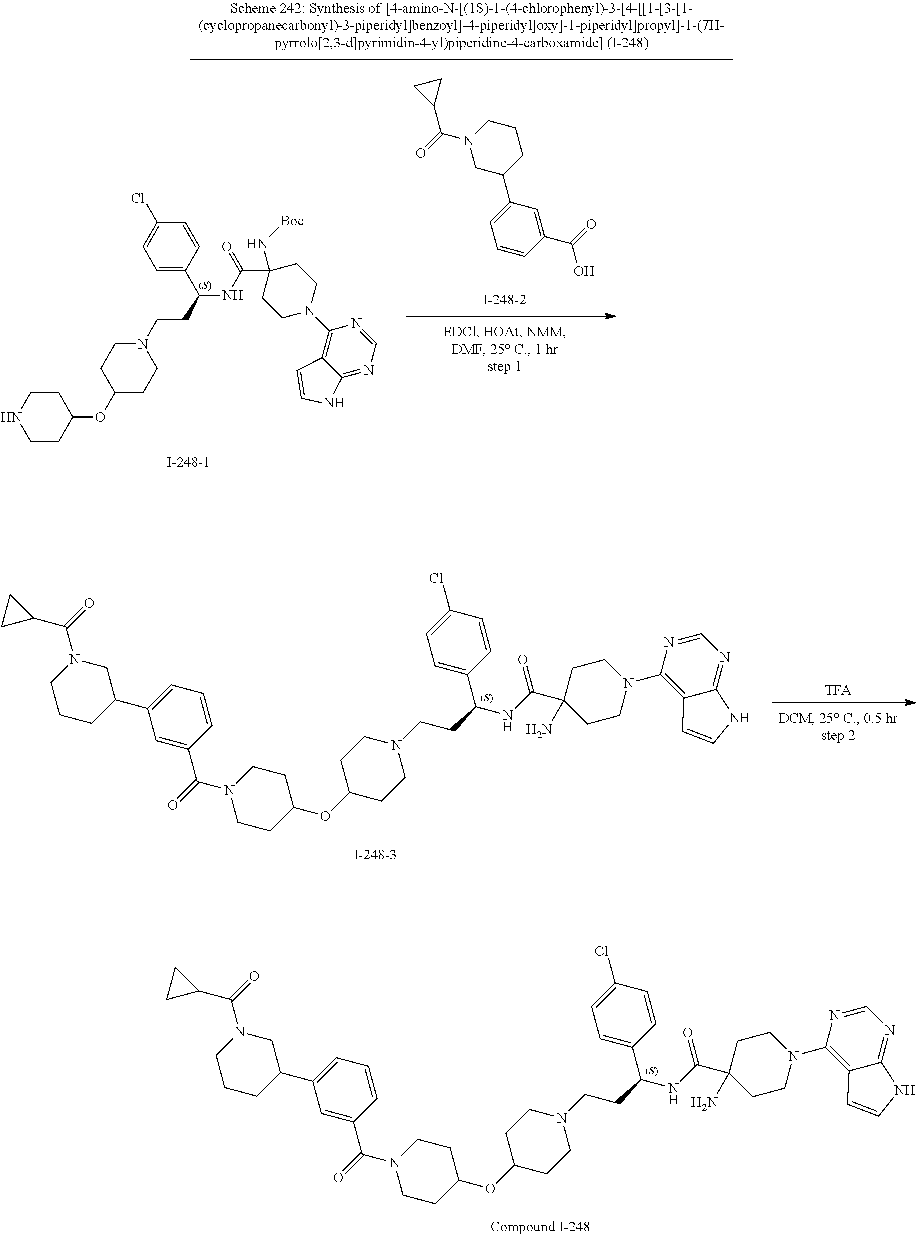

### Step 1: Synthesis of I-248-3.

To a solution of I-248-1 (60 mg, 86.29 μmol, 1 eq) and I-248-2 (34.73 mg, 86.29 μmol, 1 eq) in DMF (0.6 mL) was added EDCI (49.63 mg, 258.88 μmol, 3 eq), HOAt (11.75 mg, 86.29 μmol, 12.07 μL, 1 eq) and NMM (43.64 mg, 431.47 μmol, 47.44 μL, 5 eq). The mixture was stirred at 25° C. for 1 hr. The mixture was quenched by H₂O (0.6 mL) at 25° C., and then extracted with DCM 0.6 mL (0.2 mL*3), dried over Na₂SO₄, filtered, and concentrated under reduced pressure to give a crude product. The crude product was purified by reversed phase column (0.1% FA condition) and lyophilized to give the product. I-248-3 (30 mg, 28.27 μmol, 32.76% yield, 89.571% purity) was obtained as a white solid. LCMS: R^{t}=0.372 min, [M+H]⁺=950.3.

### Step 2: Synthesis of I-248.

To a solution of I-248-3 (20 mg, 21.04 μmol, 1 eq) in DCM (0.5 mL) was added TFA (0.1 mL). The mixture was stirred at 25° C. for 0.5 hr. The mixture was dried with N₂ to give a crude product. The crude product was purified by reversed phase column (0.1% TFA condition) and lyophilized to give the product. I-248 (11.77 mg, 11.41 μmol, 54.22% yield, 93.478% purity, TFA) was obtained as a white solid. LCMS: R^{t}=0.314 min, [M+H]⁺=850.4. SFC: R^{t}=1.607 min, 1.764 min. ¹H NMR (400 MHZ, METHANOL-d4) δ=8.36 (s, 1H), 7.47-7.24 (m, 9H), 6.91 (d, J=3.6 Hz, 1H), 5.02-4.98 (m, 1H), 4.65-4.50 (m, 3H), 4.40-4.33 (m, 1H), 4.07-3.94 (m, 1H), 3.93-3.74 (m, 4H), 3.57 (s, 3H), 3.37 (s, 1H), 3.24-3.19 (m, 3H), 3.12-2.91 (m, 2H), 2.90-2.77 (m, 1H), 2.76-2.65 (m, 2H), 2.65-2.56 (m, 1H), 2.40-2.28 (m, 1H), 2.27-2.08 (m, 3H), 2.07-1.79 (m, 10H), 1.71-1.42 (m, 4H), 0.90-0.74 (m, 4H). ¹⁹F NMR (376 MHz, METHANOL-d4) δ=−77.029.

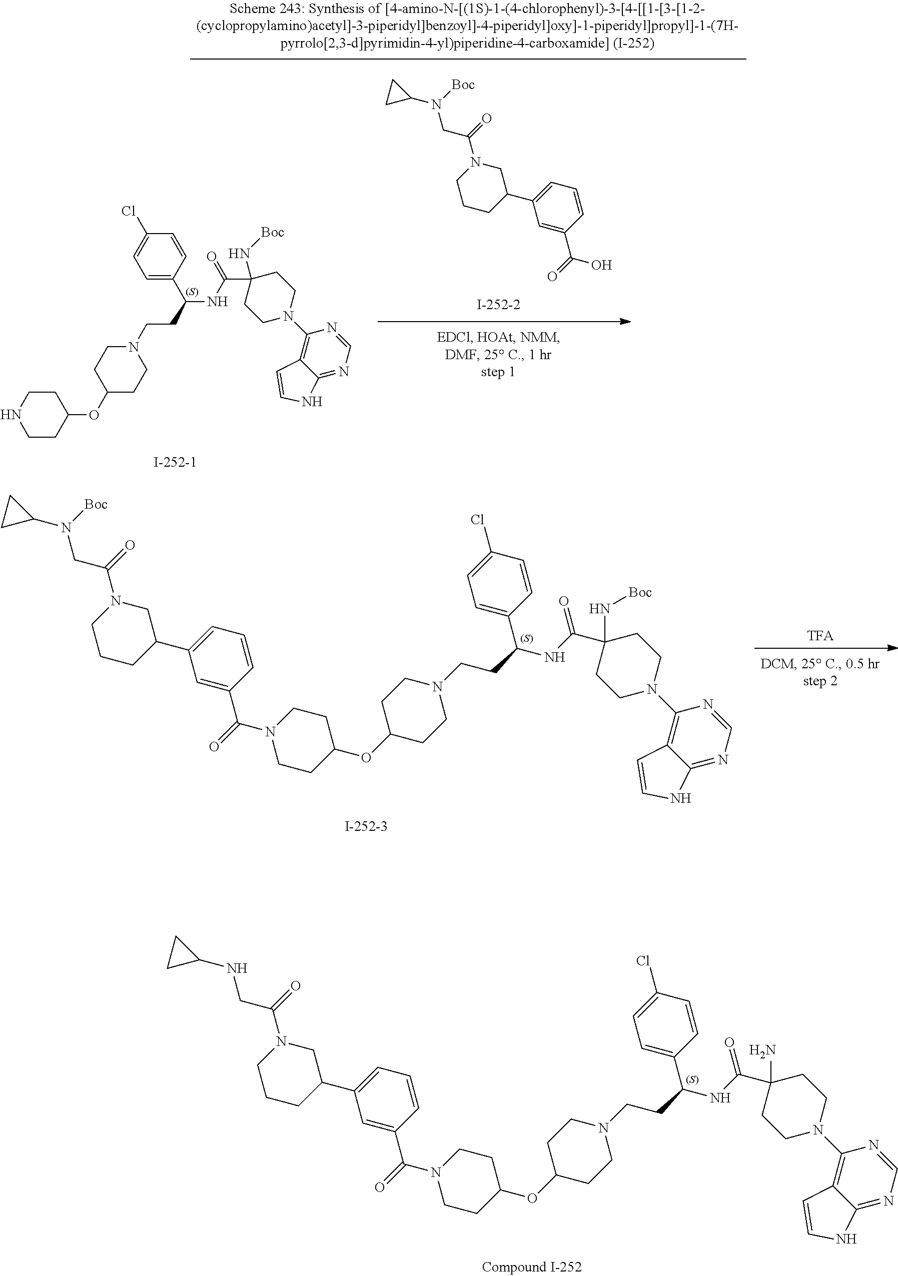

### Step 1: Synthesis of I-252-3.

To a solution of I-252-1 (60 mg, 86.29 μmol, 1 eq) and I-252-2 (34.73 mg, 86.29 μmol, 1 eq) in DMF (0.6 mL) was added EDCI (49.63 mg, 258.88 μmol, 3 eq), HOAt (11.75 mg, 86.29 μmol, 12.07 μL, 1 eq) and NMM (43.64 mg, 431.47 μmol, 47.44 μL, 5 eq). The mixture was stirred at 25° C. for 1 hr. The mixture was quenched by addition H₂O (0.6 mL) at 25° C., and extracted with DCM 0.6 mL (0.2 mL*3), dried over Na₂SO₄, filtered, and concentrated under reduced pressure to give a crude product. The crude product was purified by reversed phase column (0.1% FA condition) and lyophilized to give the product. I-252-3 (40 mg, 34.81 μmol, 40.34% yield, 93.974% purity) was obtained as a white solid. LCMS: R^{t}=0.450 min, [M+H]⁺=1079.4.

### Step 2: Synthesis of I-252.

To a solution of I-252-3 (30 mg, 27.78 μmol, 1 eq) in DCM (0.5 mL) was added TFA (153.50 mg, 1.35 mmol, 0.1 mL, 48.45 eq). The mixture was stirred at 25° C. for 0.5 hr. The mixture was dried with N₂, and then added 10 mL H₂O and lyophilized to give the product. I-252 (25.42 mg, 25.58 μmol, 92.09% yield, 100% purity, TFA) was obtained as a white solid. LCMS: R^{t}=0.270 min, [M+H]⁺=879.4. SFC: R^{t}=2.893 min, 3.514 min. ¹H NMR (400 MHZ, METHANOL-d4) δ=8.38 (s, 1H), 7.48-7.24 (m, 9H), 6.93 (d, J=3.6 Hz, 1H), 5.02-4.98 (m, 1H), 4.65-4.50 (m, 3H), 4.33-4.25 (m, 1H), 4.24-4.12 (m, 1H), 4.01 (s, 1H), 3.93-3.73 (m, 5H), 3.68-3.45 (m, 3H), 3.37 (s, 1H), 3.24-3.15 (m, 3H), 3.13-2.94 (m, 2H), 2.93-2.84 (m, 1H), 2.83-2.68 (m, 3H), 2.66-2.57 (m, 1H), 2.38-2.12 (m, 4H), 2.08-1.78 (m, 9H), 1.76-1.50 (m, 4H), 0.94-0.87 (m, 4H), 1′F NMR (376 MHz, METHANOL-d4) δ=−77.112.

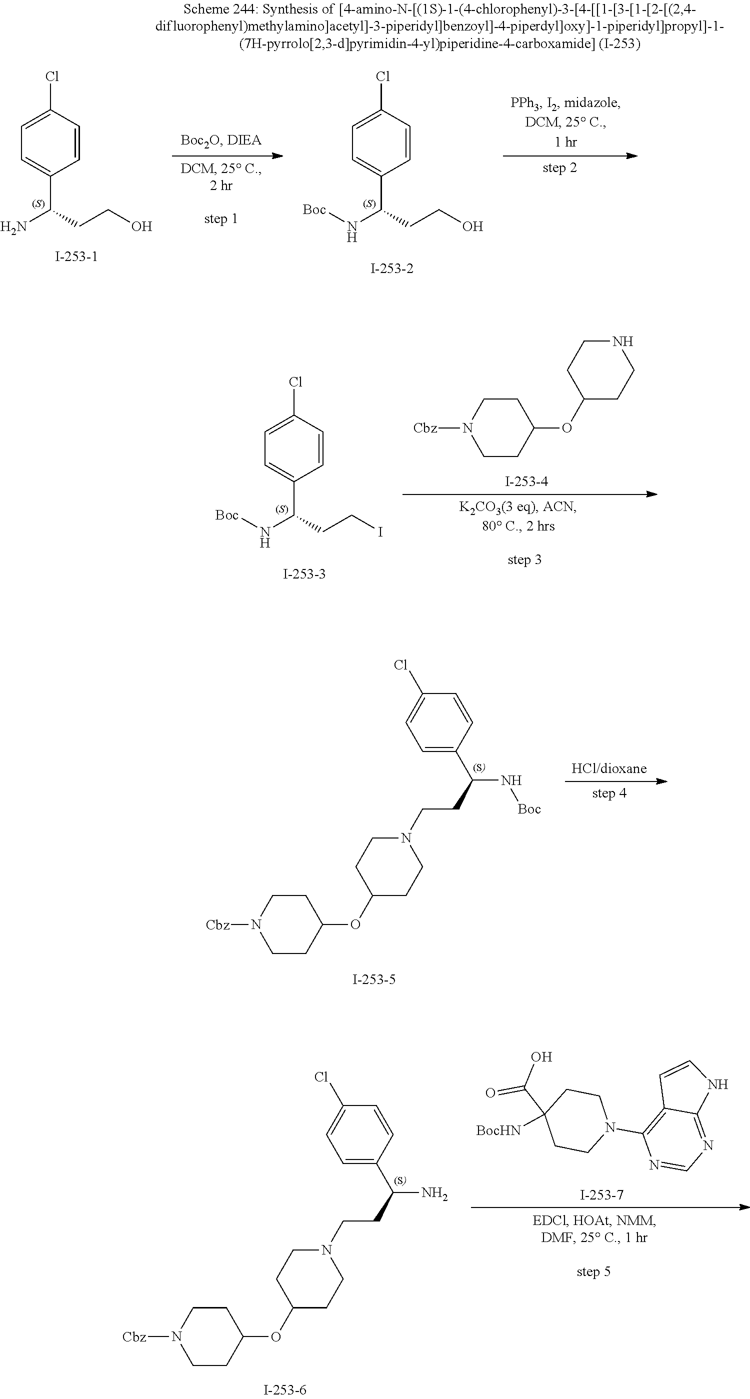

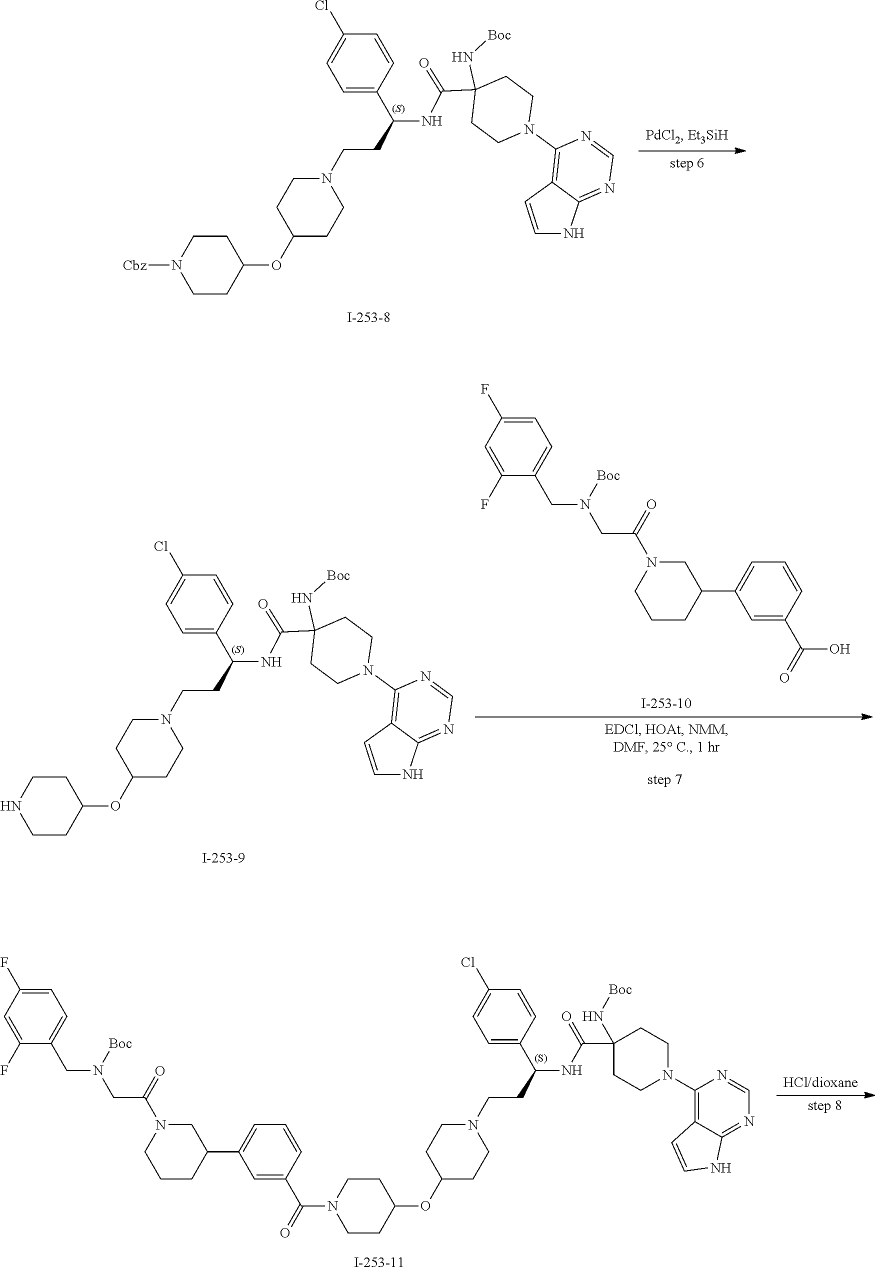

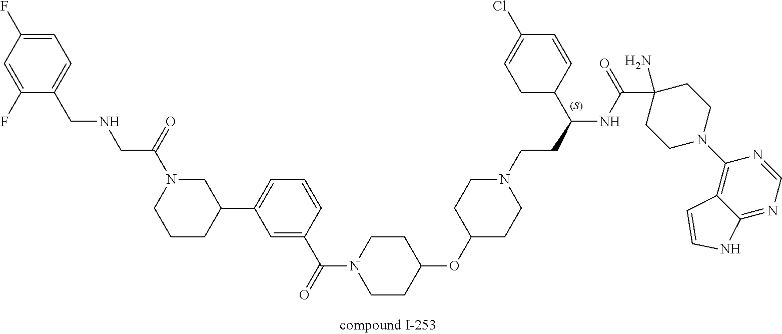

### Step 1: Synthesis of I-253-2

To a solution of I-253-1 (50 g, 269.32 mmol, 1 eq) in DCM (500 mL) was added (Boc) 20 (70.53 g, 323.19 mmol, 74.25 mL, 1.2 eq) and DIEA (104.42 g, 807.97 mmol, 140.73 mL, 3 eq). The mixture was stirred at 25° C. for 1 hr. The mixture was quenched with water (100 mL) and extracted with DCM (150 mL*3). The combined organic layers dried over sodium sulfate, filtered, and concentrated under reduced pressure to give a residue. The residue was triturated with PE (90 mL), the suspension was stirred at 25° C. for 0.5 hr and filtered to give product. I-253-2 (50 g, 174.97 mmol, 64.97% yield) was obtained as a white solid.

### Step 2: Synthesis of I-253-3

To a solution of PPh₃ (36.71 g, 139.97 mmol, 2 eq) and imidazole (19.06 g, 279.95 mmol, 4 eq) in DCM (400 mL) was added I2 (35.53 g, 139.97 mmol, 28.20 mL, 2 eq) at 0° C. The mixture was stirred at 0° C. for 0.5 hr. Then a mixture of I-253-2 (20 g, 69.99 mmol, 1 eq) in DCM (200 mL) was added into the reaction mixture. The reaction mixture was stirred at 25° C. for 11.5 hrs. The mixture was quenched with aq. Na₂CO₃ (1 L) and extracted with DCM (500 mL*3). The combined organic layers dried over sodium sulfate, filtered, and concentrated under reduced pressure to give a residue. The crude product was purified by column chromatography (SiO₂, PE/EA=1/0 to 0/1) and the eluent was concentrated under reduced pressure to give product. I-253-3 (20 g, 50.55 mmol, 72.22% yield) was obtained as a white solid.

### Step 3: Synthesis of I-253-5

To a solution of I-253-3 (1 g, 2.53 mmol, 1 eq) in ACN (10 mL) was added K₂CO₃ (1.05 g, 7.58 mmol, 3 eq) and I-253-4 (986.59 mg, 2.78 mmol, 1.1 eq, HCl). The mixture was stirred at 80° C. for 2 hrs. The reaction mixture was quenched with H₂O (20 mL) at 25° C., and then extracted with EA 60 ml (20 ml*3), and combined organic phase was dried with anhydrous sodium sulfate, filtered, and filtrate was concentrated to give crude product. The crude product was purified by column chromatography (SiO₂, EA/MeOH=1/0 to 0/1) and the eluent was concentrated under reduced pressure to give I-253-5 (1 g, 1.30 mmol, 51.30% yield, 76% purity), which was obtained as a yellow oil. LCMS: R^{t}=0.421 min, [M+H]⁺=586.3. SFC: R^{t}=1.616 min.

### Step 4: Synthesis of I-253-6

To a solution of I-253-5 (1 g, 1.71 mmol, 1 eq) in HCl/dioxane (2 M, 10.00 mL, 11.72 eq) was stirred at 25° C. for 0.5 h. Concentrated under reduced pressure to give I-253-6 (900 mg, crude, HCl), which was obtained as a yellow solid. LCMS: R^{t}=0.274 min, [M+H]⁺=486.2. SFC: R^{t}=1.860 min.

### Step 5: Synthesis of I-253-8

To a solution of I-253-6 (800 mg, 1.18 mmol, 1 eq, HCl) in DMF (8 mL) was added EDCI (678.01 mg, 3.54 mmol, 3 eq), NMM (596.23 mg, 5.89 mmol, 648.07 μL, 5 eq), I-253-7 (426.06 mg, 1.18 mmol, 1 eq) and HOAt (160.47 mg, 1.18 mmol, 164.92 μL, 1 eq). The mixture was stirred at 25° C. for 0.5 h. The reaction mixture was quenched with H₂O (50 mL) at 25° C., and then the mixture was extracted with EA 150 ml (50 ml*3), and combined organic phase was dried with anhydrous sodium sulfate, filtered, and filtrate was concentrated to give crude product. The crude product was purified by reversed phase column (0.1% FA condition) and the eluent was lyophilized to give I-253-8 (950 mg, 1.07 mmol, 90.35% yield, 93% purity), which was obtained as a white solid. LCMS: R^{t}=0.363 min, [M+H]⁺=829.2. SFC: R^{t}=2.293 min.

### Step 6: Synthesis of I-253-9

To a solution of I-253-8 (850 mg, 1.02 mmol, 1 eq) in DCM (8.5 mL) was added PdCl2 (90.86 mg, 512.40 μmol, 0.5 eq), Et3SiH (1.43 g, 12.30 mmol, 1.96 mL, 12 eq) and TEA (311.10 mg, 3.07 mmol, 427.92 μL, 3 eq). The mixture was stirred at 25° C. for 4 hrs. The mixture was filtered, and concentrated under reduced pressure to give a residue. The crude product was purified by reversed phase column (0.1% FA condition) and the eluent was lyophilized to give I-253-9 (340 mg, 479.22 μmol, 46.76% yield, 98% purity), which was obtained as a white solid. LCMS: R^{t}=0.269 min, [M+H]⁺=695.3. SFC: R^{t}=1.937 min.

### Step 7: Synthesis of I-253-11

To a solution of I-253-9 (70 mg, 100.68 μmol, 1 eq) in DMF (0.7 mL) was added EDCI (57.90 mg, 302.03 μmol, 3 eq), NMM (50.92 mg, 503.39 μmol, 55.34 μL, 5 eq) and I-253-10 (49.18 mg, 100.68 μmol, 1 eq) and HOAt (13.70 mg, 100.68 μmol, 14.08 μL, 1 eq). The mixture was stirred at 25° C. for 0.5 h. The mixture was purified by reversed phase column (0.1% FA condition) and the eluent was lyophilized to give I-253-11 (60 mg, 47.35 μmol, 47.03% yield, 92% purity), which was obtained as a white solid. LCMS: R^{t}=0.480 min, [M/2+H]⁺=583.8. SFC: R^{t}=1.530 min.

### Step 8: Synthesis of I-253

To a solution of I-253-11 (50 mg, 42.89 μmol, 1 eq) in HCl/dioxane (2 M, 500.00 μL, 23.32 eq) was stirred at 25° C. for 0.5 h. Concentrated under reduced pressure to give a residue. Then add 10 ml H₂O into the residue, and then was lyophilized to give I-253 (29.68 mg, 30.74 μmol, 71.67% yield, 100% purity), which was obtained as a white solid. LCMS: R^{t}=0.693 min, [M+H]⁺=1965.6. SFC: R^{t}=2.019 min, 2.284 min. ¹H NMR (400 MHZ, METHANOL-d4) δ=8.39 (s, 1H), 7.71-7.61 (m, 1H), 7.49-7.40 (m, 5H), 7.39-7.32 (m, 3H), 7.29 (t, J=5.6 Hz, 1H), 7.19-7.07 (m, 2H), 7.02 (d, J=2.8 Hz, 1H), 5.04-5.02 (m, 1H), 4.68-4.49 (m, 3H), 4.37-4.30 (m, 2H), 4.29-4.09 (m, 2H), 4.09-3.88 (m, 4H), 3.88-3.67 (m, 3H), 3.67-3.50 (m, 3H), 3.45-3.37 (m, 1H), 3.29-3.19 (m, 3H), 3.18-3.04 (m, 2H), 2.86 (t, J=12.0 Hz, 2H), 2.82-2.69 (m, 2H), 2.58-2.42 (m, 1H), 2.37-2.14 (m, 3H), 2.12-1.96 (m, 5H), 1.93-1.80 (m, 3H), 1.79-1.44 (m, 4H). ¹⁹F NMR (400 MHZ, METHANOL-d4) δ=−128, −132.

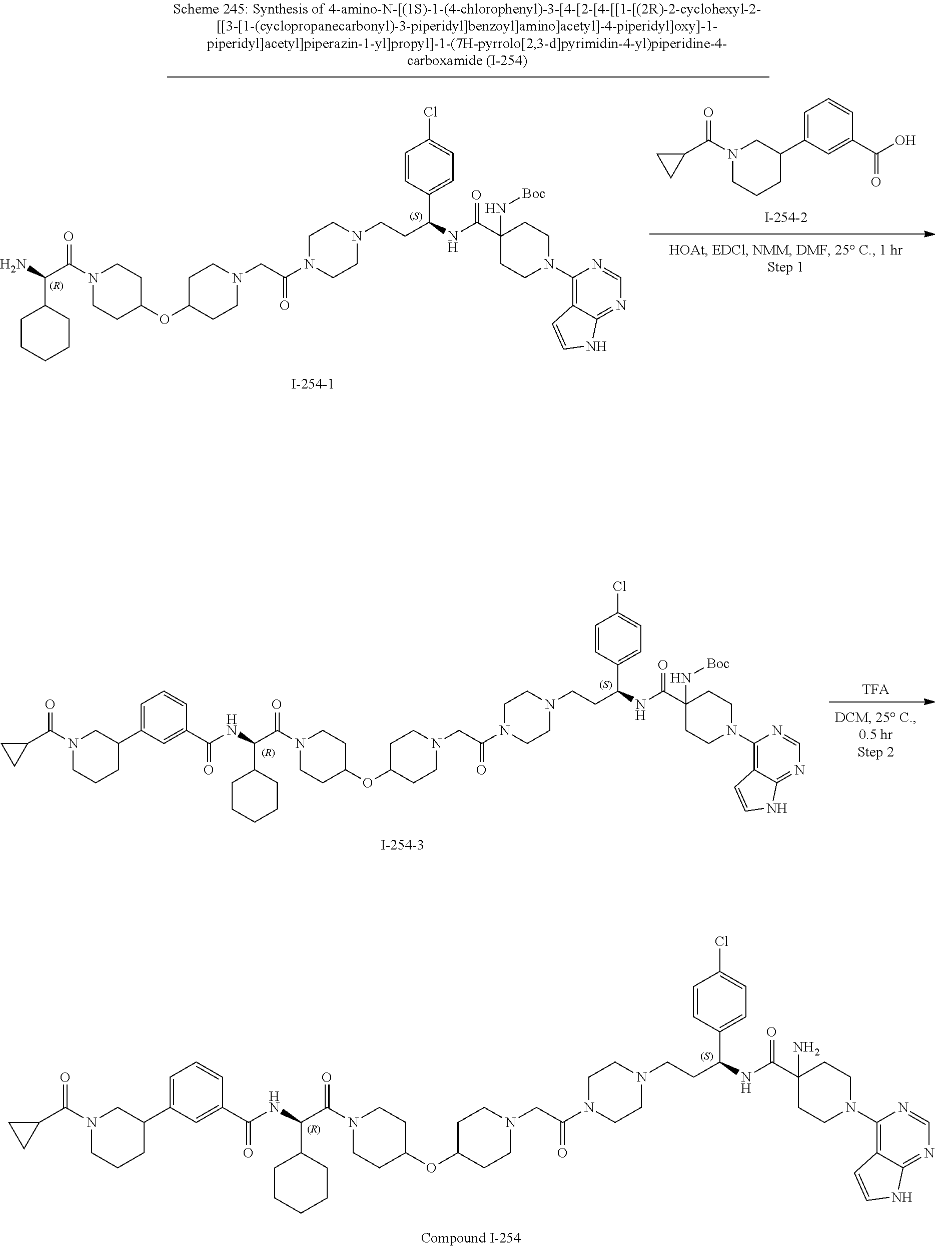

### Step 1: Synthesis of I-254-3

To a solution of I-254-1 (30 mg, 31.23 μmol, 1 eq) and I-254-2 (9.39 mg, 34.35 μmol, 1.1 eq) in DMF (0.5 mL) was added EDCI (17.96 mg, 93.69 μmol, 3 eq) and HOAt (4.25 mg, 31.23 μmol, 4.37 μL, 1 eq), NMM (15.79 mg, 156.15 μmol, 17.17 μL, 5 eq). The mixture was stirred at 25° C. for 1 hr. The mixture was diluted with water (5 mL) and extracted with DCM (5 mL*3). The combined organic layer was dried over Na₂SO₄, filtered, and concentrated in vacuo to give a residue. The residue was purified by prep-HPLC (column: CD02-Waters Xbidge BEH C18 150*25*10 um; mobile phase: [water (NH4HCO3)-ACN]; gradient: 44%-74% B over 10 min). Then eluent was concentrated in vacuo and lyophilized. I-254-3 (30 mg, 23.93 μmol, 76.63% yield, 97% purity) was obtained as a white solid. LCMS: R^{t}=0.379 min, [M+H]⁺=1215.3. R^{t}=7.227 min, 9.026 min.

### Step 2: Synthesis of I-254

To a solution of I-254-3 (30 mg, 24.67 μmol, 1 eq) in DCM (0.5 mL) was added TFA (153.50 mg, 1.35 mmol, 0.1 mL, 54.56 eq). The mixture was stirred at 25° C. for 0.5 hr. The reaction mixture was concentrated in vacuo to give a residue. The residue was purified by prep-HPLC (column: CD02-Waters Xbidge BEH C18 150*25*10 um; mobile phase: [water (NH₄HCO₃)-ACN]; gradient: 38%-68% B over 10 min). Then eluent was concentrated in vacuo and lyophilized. I-254 (6.48 mg, 5.23 μmol, 21.21% yield, 99.311% purity, TFA) was obtained as a white solid. LCMS: R^{t}=0.678 min, [M+H]⁺=1115.7. SFC: R^{t}=1.700 min, 2.017 min. ¹H NMR (400 MHZ, METHANOL-d4) δ=8.38 (s, 1H), 7.81-7.66 (m, 2H), 7.55-7.41 (m, 2H), 7.39-7.34 (m, 5H), 6.93 (d, J=3.6 Hz, 1H), 5.03 (t, J=6.4 Hz, 1H), 4.93 (s, 1H), 4.68-4.55 (m, 3H), 4.45-4.36 (m, 1H), 4.33-4.26 (m, 2H), 4.05-4.01 (m, 1H), 3.95 (s, 1H), 3.90-3.75 (m, 5H), 3.75-3.62 (m, 3H), 3.58-3.46 (m, 2H), 3.46-3.34 (m, 5H), 3.29-3.19 (m, 3H), 3.18-3.09 (m, 2H), 2.91-2.77 (m, 1H), 2.75-2.57 (m, 3H), 2.43-2.27 (m, 2H), 2.18 (d, J=14.4 Hz, 2H), 2.04 (d, J=12.4 Hz, 6H), 1.97-1.85 (m, 6H), 1.83-1.75 (m, 3H), 1.68 (d, J=10.4 Hz, 3H), 1.60-1.53 (m, 1H), 1.35-1.22 (m, 4H), 1.17-1.05 (m, 2H), 0.92-0.78 (m, 4H), 19 F NMR (400 MHZ, METHANOL-d4) δ=−77.142.

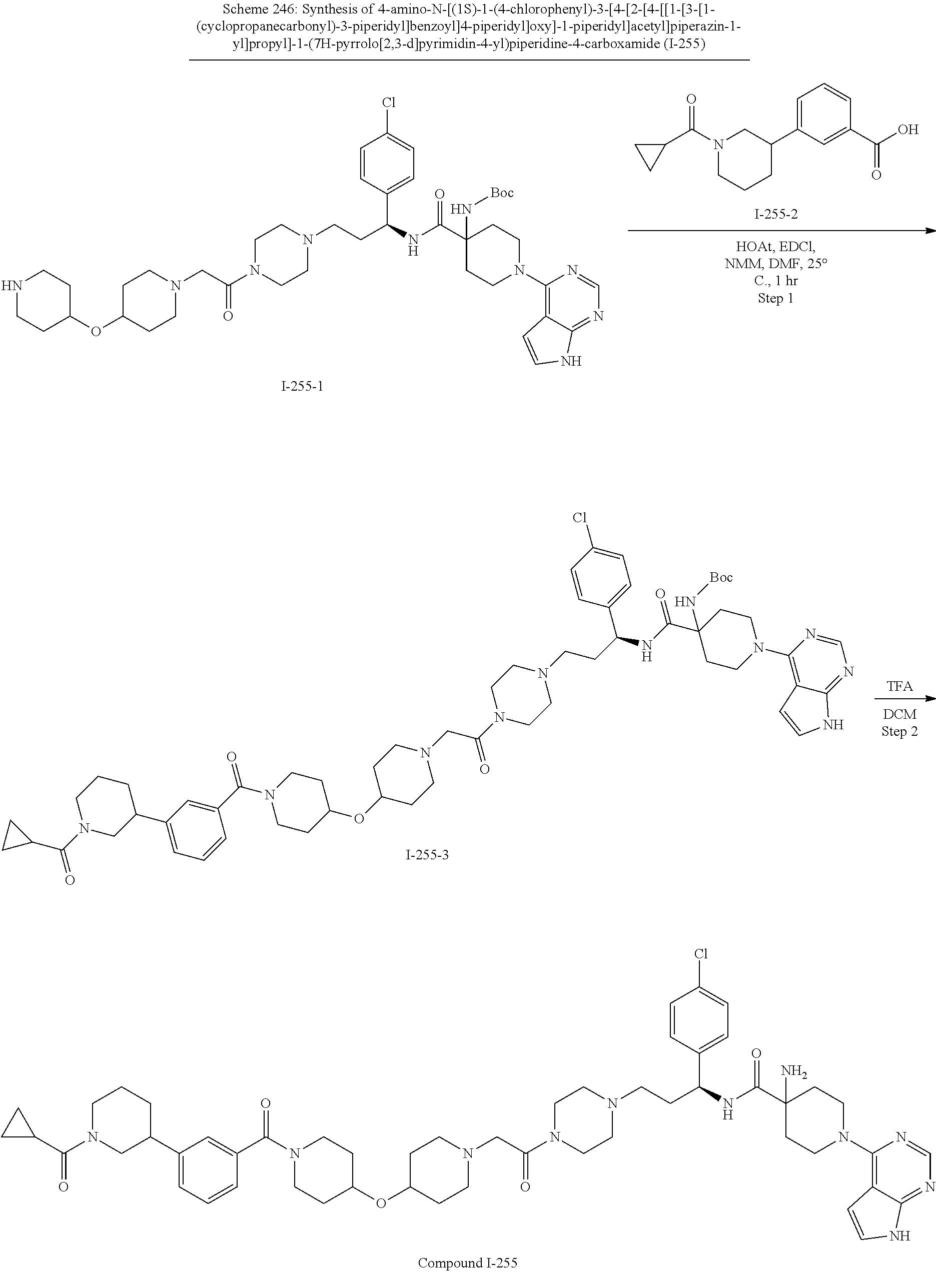 [SMILES: CC(C)(C)OC(=O)NC1(C(=O)N[C@@H](CCN2CCN(C(=O)CN3CCC(OC4CCNCC4)CC3)CC2)c2ccc(Cl)cc2)CCN(c2ncnc3[nH]ccc23)CC1 ; "TFA" ; O=C(O)c1cccc(C2CCCN(C(=O)C3CC3)C2)c1 ; CC(C)(C)OC(=O)NC1(C(=O)N[C@@H](CCN2CCN(C(=O)CN3CCC(OC4CCN(C(=O)c5cccc(C6CCCN(C(=O)C7CC7)C6)c5)CC4)CC3)CC2)c2ccc(Cl)cc2)CCN(c2ncnc3[nH]ccc23)CC1 ; NC1(C(=O)N[C@@H](CCN2CCN(C(=O)CN3CCC(OC4CCN(C(=O)c5cccc(C6CCCN(C(=O)C7CC7)C6)c5)CC4)CC3)CC2)c2ccc(Cl)cc2)CCN(c2ncnc3[nH]ccc23)CC1]

### Step 1: Synthesis of I-255-3

To a solution of I-255-2 (30 mg, 109.76 μmol, 1 eq) and I-255-1 (99.18 mg, 120.73 μmol, 1.1 eq) in DMF (0.5 mL) was added EDCI (63.12 mg, 329.28 μmol, 3 eq) and HOAt (14.94 mg, 109.76 μmol, 15.35 μL, 1 eq), NMM (55.51 mg, 548.79 μmol, 60.34 μL, 5 eq). The mixture was stirred at 25° C. for 1 hr.

The mixture was diluted with water (5 mL) and extracted with DCM (5 mL*3). The combined organic layer was dried over Na₂SO₄, filtered, and concentrated in vacuo to give a residue. The residue was purified by prep-HPLC (column: CD02-Waters Xbidge BEH C18 150*25*10 um; mobile phase: [water (NH₄HCO₃)-ACN]; gradient: 36%-66% B over 10 min). Then eluent was concentrated in vacuo and lyophilized. I-255-3 (70 mg, 64.36 μmol, 58.64% yield, 99% purity) was obtained as a white solid. LCMS: R^{t}=0.324 min, [M+H]⁺=1076.4. SFC: R^{t}=2.126 min, 3.959 min.

### Step 2: Synthesis of I-255

To a solution of I-255-3 (50 mg, 46.44 μmol, 1 eq) in DCM (0.5 mL) was added TFA (383.75 mg, 3.37 mmol, 250.00 μL, 72.48 eq). The mixture was stirred at 25° C. for 0.5 hr. The reaction mixture was concentrated in vacuo to give a residue. The residue was used into lyophilized directly without further purification. I-255 (49.84 mg, 44.01 μmol, 94.78% yield, 96.313% purity, TFA) was obtained as a white solid. R^{t}=0.287 min, [M+H]⁺=976.4. R^{t}=3.323 min, 5.082 min. ¹H NMR (400 MHZ, METHANOL-d4) δ=8.39 (s, 1H), 7.46-7.40 (m, 2H), 7.39 (d, J=3.6 Hz, 1H), 7.36 (d, J=3.6 Hz, 4H), 7.33 (d, J=6.4 Hz, 1H), 7.30-7.24 (m, 1H), 6.95 (d, J=3.6 Hz, 1H), 5.03-5.01 (m, 1H), 4.66-4.59 (m, 2H), 4.55 (d, J=10.4 Hz, 1H), 4.38 (d, J=11.6 Hz, 1H), 4.30 (s, 2H), 4.01-4.00 (m, 1H), 3.96-3.82 (m, 4H), 3.81-3.70 (m, 3H), 3.67-3.52 (m, 3H), 3.44 (s, 2H), 3.41-3.32 (m, 4H), 3.29-3.23 (m, 2H), 3.20-3.12 (m, 2H), 2.80 (s, 1H), 2.78-2.54 (m, 4H), 2.44-2.27 (m, 2H), 2.26-2.15 (m, 2H), 2.05 (d, J=11.6 Hz, 6H), 1.97-1.89 (m, 2H), 1.88-1.78 (m, 3H), 1.72-1.53 (m, 3H), 0.92-0.79 (m, 4H). ¹⁹F NMR (400 MHZ, METHANOL-d4) δ=−77.157.

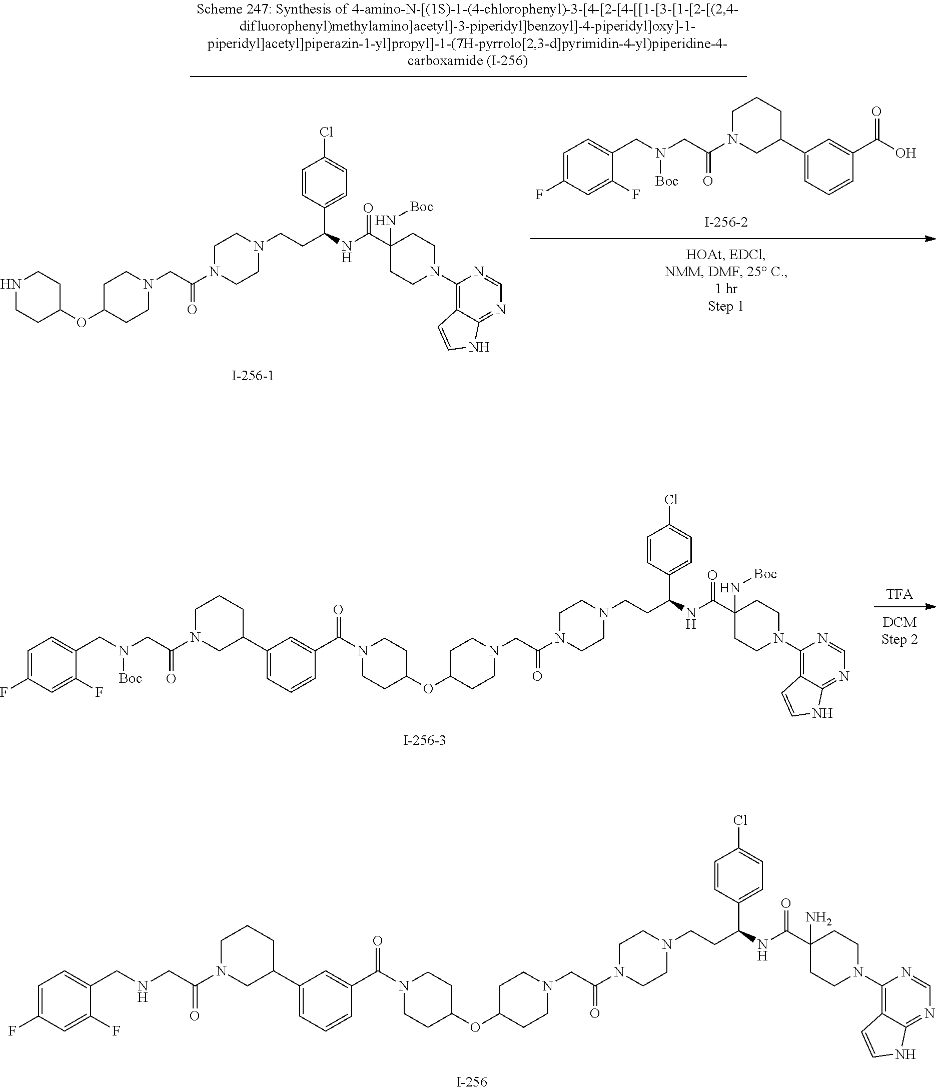 [SMILES: "TFA" ; CC(C)(C)OC(=O)NC1(C(=O)N[C@@H](CCN2CCN(C(=O)CN3CCC(OC4CCNCC4)CC3)CC2)c2ccc(Cl)cc2)CCN(c2ncnc3[nH]ccc23)CC1 ; CC(C)(C)OC(=O)N(CC(=O)N1CCCC(c2cccc(C(=O)O)c2)C1)Cc1ccc(F)cc1F ; CC(C)(C)OC(=O)NC1(C(=O)N[C@@H](CCN2CCN(C(=O)CN3CCC(OC4CCN(C(=O)c5cccc(C6CCCN(C(=O)CN(Cc7ccc(F)cc7F)C(=O)OC(C)(C)C)C6)c5)CC4)CC3)CC2)c2ccc(Cl)cc2)CCN(c2ncnc3[nH]ccc23)CC1 ; NC1(C(=O)N[C@@H](CCN2CCN(C(=O)CN3CCC(OC4CCN(C(=O)c5cccc(C6CCCN(C(=O)CNCc7ccc(F)cc7F)C6)c5)CC4)CC3)CC2)c2ccc(Cl)cc2)CCN(c2ncnc3[nH]ccc23)CC1]

### Step 1: Synthesis of I-256-3

To a solution of I-256-1 (50 mg, 60.87 μmol, 1 eq) and I-256-2 (35.68 mg, 73.04 μmol, 1.2 eq) in DMF (0.5 mL) was added EDCI (35.01 mg, 182.60 μmol, 3 eq) and HOAt (8.28 mg, 60.87 μmol, 8.51 μL, 1 eq), NMM (30.78 mg, 304.34 μmol, 33.46 μL, 5 eq). The mixture was stirred at 25° C. for 1 hr. The mixture was diluted with water (5 mL) and extracted with DCM (5 mL*3). The combined organic layer was dried over Na₂SO₄, filtered, and concentrated in vacuo to give a residue. The residue was purified by prep-HPLC (column: CD01-Phenomenex luna C18 150*25*10 um; mobile phase: [water (TFA)-ACN]; gradient: 20%-50% B over 4 min). Then eluent was concentrated in vacuo and lyophilized. I-256-3 (60 mg, 44.58 μmol, 73.25% yield, 96% purity) was obtained as a white solid. LCMS: R^{t}=0.426 min, [M/2+H]⁺=646.9. R^{t}=3.064 min.

### Step 2: Synthesis of I-256

To a solution of I-256-3 (40 mg, 30.96 μmol, 1 eq) in DCM (0.3 mL) was added TFA (153.50 mg, 1.35 mmol, 0.1 mL, 43.48 eq). The mixture was stirred at 25° C. for 0.5 hr. The reaction mixture was concentrated in vacuo to give a residue. The residue was used into lyophilized directly without further purification. I-256 (35.66 mg, 29.58 μmol, 95.52% yield, 100% purity, TFA) was obtained as a white solid. LCMS: R^{t}=0.267 min, [M+H]⁺=1091.3. SFC: R^{t}=4.488 min, 4.765 min. ¹H NMR (400 MHZ, METHANOL-d4) δ=8.39 (s, 1H), 7.66-7.58 (m, 1H), 7.46-7.41 (m, 2H), 7.38 (d, J=3.6 Hz, 1H), 7.36 (d, J=3.6 Hz, 4H), 7.33 (s, 1H), 7.32-7.28 (m, 1H), 7.17-7.07 (m, 2H), 6.94 (d, J=3.6 Hz, 1H), 5.03-5.01 (m, 1H), 4.65-4.59 (m, 2H), 4.57-4.50 (m, 1H), 4.31 (d, J=10.4 Hz, 4H), 4.28-4.22 (m, 1H), 4.18 (d, J=11.6 Hz, 1H), 4.14-4.06 (m, 1H), 4.03 (s, 1H), 3.95-3.91 (m, 1H), 3.87-3.85 (m, 2H), 3.84-3.77 (m, 2H), 3.72 (s, 3H), 3.67-3.54 (m, 3H), 3.50-3.43 (m, 2H), 3.39 (s, 2H), 3.34-3.31 (m, 1H), 3.30-3.26 (m, 2H), 3.25-3.09 (m, 4H), 2.88-2.82 (m, 1H), 2.78-2.70 (m, 1H), 2.70-2.57 (m, 2H), 2.43-2.34 (m, 1H), 2.31-2.25 (m, 1H), 2.23-2.14 (m, 2H), 2.10-2.01 (m, 5H), 1.92-1.80 (m, 4H), 1.74-1.62 (m, 2H), 1.59-1.52 (m, 1H). ¹⁹F NMR (400 MHZ, METHANOL-d4) δ=−77.082, −108.433, −112.876.

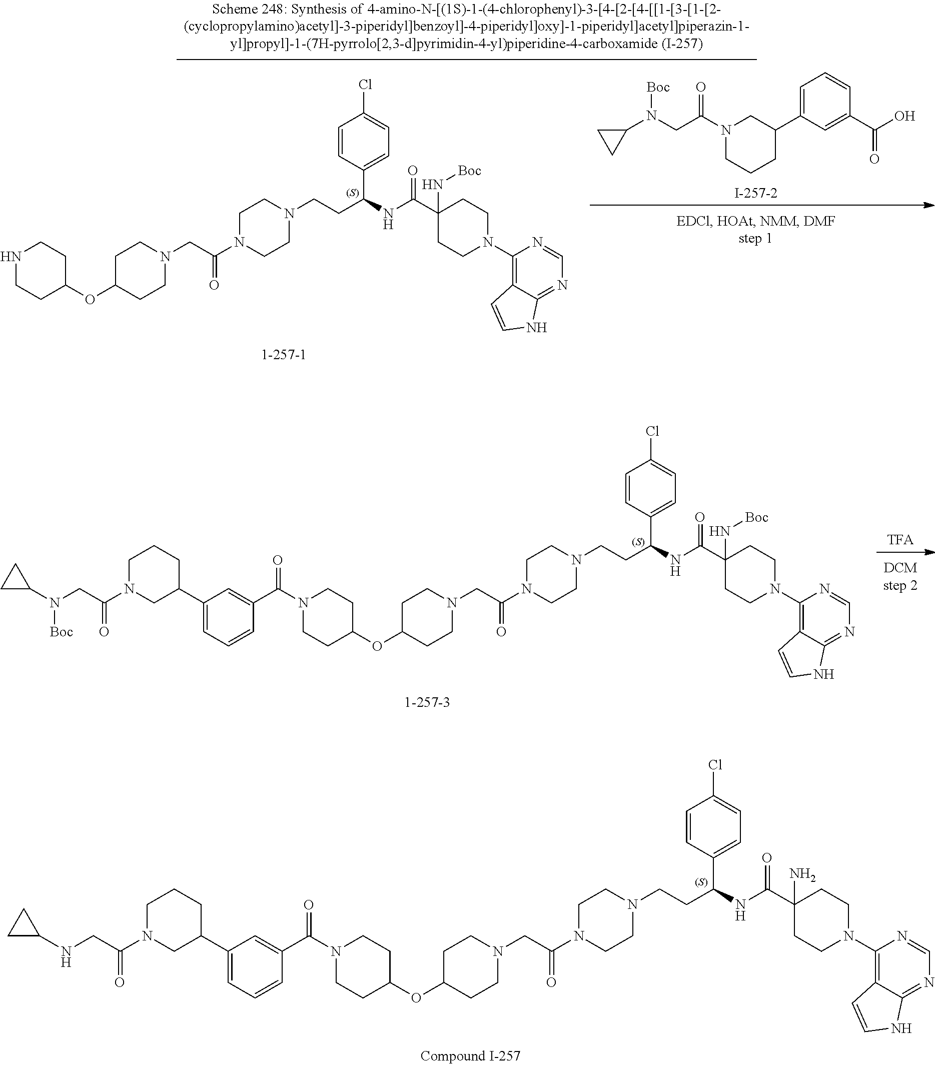

### Step 1: Synthesis of I-257-3

To a solution of I-257-1 (25 mg, 62.11 μmol, 1 eq) in DMF (0.5 mL) was added HOAt (8.45 mg, 62.11 μmol, 8.69 μL, 1 eq), EDCI (35.72 mg, 186.34 μmol, 3 eq), NMM (31.41 mg, 310.57 μmol, 34.15 μL, 5 eq) and I-257-2 (51.02 mg, 62.11 μmol, 1 eq). The mixture was stirred at 25° C. for 1 hr. The residue was diluted with H₂O 5 mL and extracted with EA 15 mL (5 mL*3), dried over Na₂SO₄, filtered, and concentrated under reduced pressure to give a residue. The residue was purified by flash silica gel chromatography (ISCO®; 4 g Silica SepaFlash® Flash Column, Eluent of 0˜100% Dichloromethane/Methanol @ 18 mL/min). The eluent was concentrated under reduced pressure to give product. I-257-3 (70 mg, 58.05 μmol, 93.45% yield) as a yellow oil. LCMS: R^{t}=0.354 min, [M+H]⁺=1205.3.

### Step 2: Synthesis of 1-257

To a solution of I-257-3 (70 mg, 58.05 μmol, 1 eq) in DCM (0.6 mL) was added TFA (307.00 mg, 2.69 mmol, 0.2 mL, 46.38 eq). The mixture was stirred at 25° C. for 3 hrs. The reaction liquid was concentrated by reducing pressure to obtain the crude product. The crude product was purified by reversed phase column (0.1% TFA condition), which was concentrated and lyophilizated to afford I-257 (17.17 mg, 15.21 μmol, 26.20% yield, 99.173% purity, TFA), which as a white solid. LCMS: R^{t}=0.243 min, [M+H]⁺=1005.4. SFC: R^{t}=5.252 min, 5.901 min. ¹H NMR (400 MHZ, METHANOL-d4) δ=8.21 (s, 1H), 7.50-7.39 (m, 2H), 7.38 (s, 1H), 7.36-7.24 (m, 5H), 7.21 (d, J=3.6 Hz, 1H), 6.70 (d, J=3.6 Hz, 1H), 5.07-4.99 (m, 1H), 4.67 (d, J=2.0 Hz, 2H), 4.60-4.50 (m, 1H), 4.35-4.25 (m, 1H), 4.22 (s, 2H), 4.20-4.11 (m, 1H), 4.09-3.97 (m, 1H), 3.90 (d, J=9.6 Hz, 1H), 3.85-3.73 (m, 3H), 3.72-3.56 (m, 5H), 3.50 (t, J=18.4 Hz, 3H), 3.43-3.34 (m, 2H), 3.30-3.12 (m, 3H), 2.97-2.56 (m, 9H), 2.51-2.38 (m, 2H), 2.21-2.03 (m, 6H), 2.02-1.76 (m, 7H), 1.73-1.50 (m, 3H), 0.98-0.86 (m, 4H). ¹⁹F NMR (400 MHZ, METHANOL-d4) δ=−76.866.

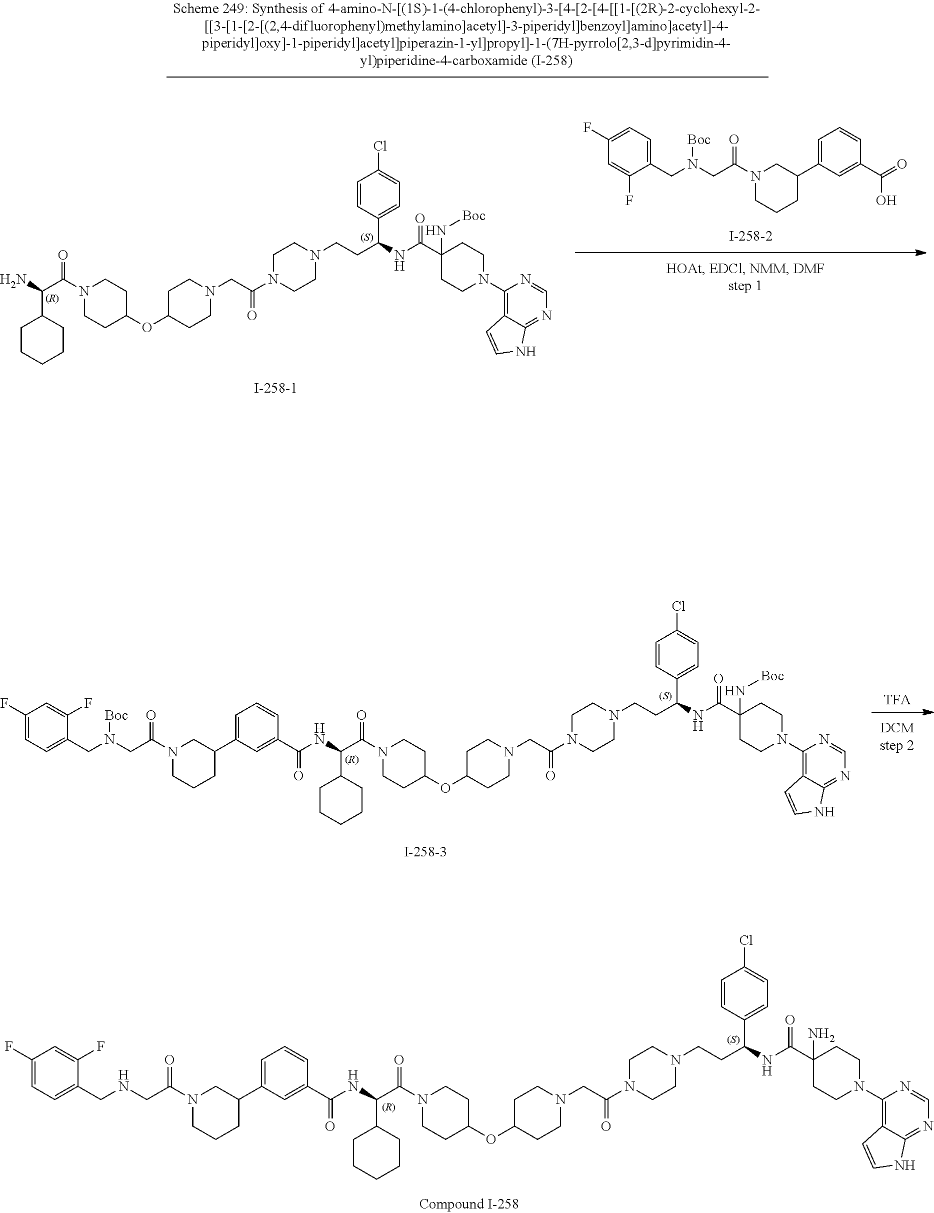

### Step 1: Synthesis of I-258-3

To a solution of I-258-2 (25 mg, 51.17 μmol, 1 eq) in DMF (0.5 mL) was added HOAt (6.97 mg, 51.17 μmol, 7.16 μL, 1 eq), EDCI (29.43 mg, 153.52 μmol, 3 eq), NMM (25.88 mg, 255.87 μmol, 28.13 μL, 5 eq) and I-258-1 (49.16 mg, 51.17 μmol, 1 eq). The mixture was stirred at 25° C. for 1 hr. The residue was diluted with H₂O 5 mL and extracted with EA 15 mL (5 mL*3), dried over Na₂SO₄, filtered, and concentrated under reduced pressure to give a residue. The residue was purified by flash silica gel chromatography (ISCO®; 4 g SepaFlash® Silica Flash Column, Eluent of 0˜100% Dichloromethane/Methanol @ 18 mL/min). The eluent was concentrated under reduced pressure to give product. I-258-3 (60 mg, 41.92 μmol, 81.92% yield) as a yellow oil. LCMS: R^{t}=0.423 min, [M+H]⁺=1430.8.

### Step 2: Synthesis of I-258

To a solution of I-258-3 (60 mg, 41.92 μmol, 1 eq) in DCM (0.6 mL) was added TFA (307.00 mg, 2.69 mmol, 0.2 mL, 64.22 eq). The mixture was stirred at 25° C. for 1 hr. The reaction liquid was concentrated by reducing pressure to obtain the crude product. The residue was purified by prep-HPLC (column: CD01-Phenomenex luna C18 150*25*10 um; mobile phase: [water (TFA)-ACN]; gradient: 15%-45% B over 10 min), which was concentrated and lyophilized to afford I-258 (32.8 mg, 23.98 μmol, 57.20% yield, 98.328% purity, TFA) as a white solid. LCMS: R^{t}=0.302 min, [M+H]⁺=1230.6. SFC: R^{t}=4.121 min, 6.876 min. ¹H NMR (400 MHZ, METHANOL-d4) δ=8.38 (s, 1H), 8.36-8.19 (m, 1H), 7.84-7.68 (m, 2H), 7.67-7.56 (m, 1H), 7.54-7.41 (m, 2H), 7.39-7.32 (m, 4H), 7.21-7.03 (m, 2H), 7.01-6.83 (m, 1H), 5.09-5.00 (m, 1H), 4.95 (d, J=3.6 Hz, 2H), 4.61 (d, J=11.6 Hz, 2H), 4.55-4.38 (m, 1H), 4.37-4.25 (m, 4H), 4.24-4.17 (m, 1H), 4.16-4.07 (m, 1H), 4.05-3.99 (m, 1H), 3.98-3.60 (m, 10H), 3.52 (d, J=12.4 Hz, 1H), 3.49-3.32 (m, 6H), 3.30-2.99 (m, 5H), 2.96-2.73 (m, 2H), 2.71-2.50 (m, 2H), 2.45-2.25 (m, 2H), 2.24-2.12 (m, 2H), 2.06 (s, 5H), 1.96-1.82 (m, 6H), 1.77 (s, 2H), 1.73-1.59 (m, 4H), 1.58-1.43 (m, 1H), 1.39-1.20 (m, 3H), 1.18-1.04 (m, 2H). ¹⁹F NMR (400 MHZ, METHANOL-d4) δ=−77.001, −108.424, −112.897.

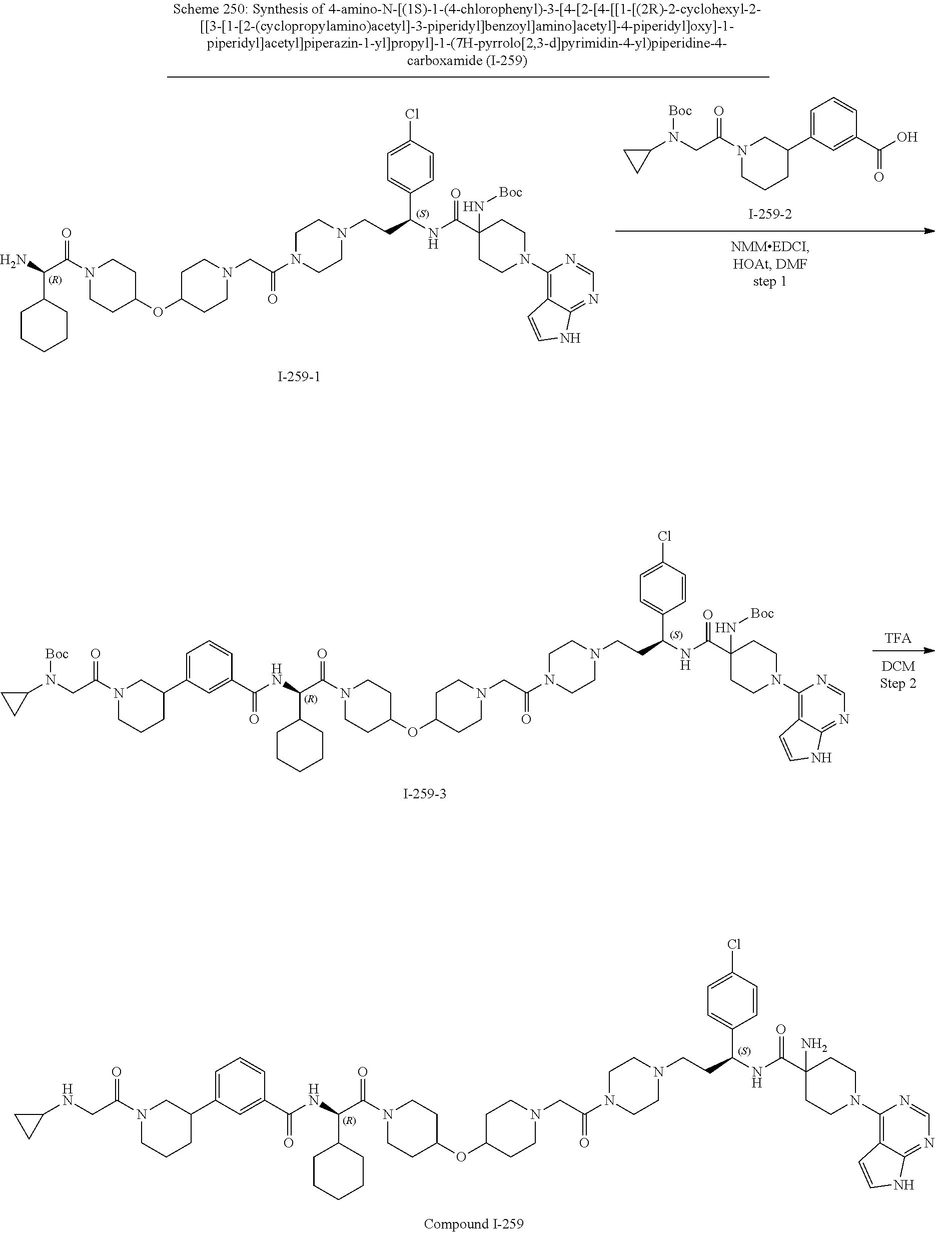

### Step 1: Synthesis of I-259-3

To a solution of I-259-2 (20 mg, 49.69 μmol, 1 eq) in DMF (0.5 mL) was added HOAt (6.76 mg, 49.69 μmol, 6.95 μL, 1 eq), EDCI (28.58 mg, 149.07 μmol, 3 eq), NMM (25.13 mg, 248.46 μmol, 27.32 μL, 5 eq) and I-259-1 (47.74 mg, 49.69 μmol, 1 eq). The mixture was stirred at 25° C. for 1 hr. The residue was diluted with H₂O 5 mL and extracted with EA 15 mL (5 mL*3), dried over Na₂SO₄, filtered, and concentrated under reduced pressure to give a residue. The residue was purified by flash silica gel chromatography (ISCO®; 4 g SepaFlash® Silica Flash Column, Eluent of 0˜100% Dichloromethane/Methanol @ 18 mL/min). The eluent was concentrated under reduced pressure to give product. I-259-3 (60 mg, 44.61 μmol, 89.77% yield) as a yellow oil. LCMS: R^{t}=0.400 min, [M+H]⁺=1344.3.

### Step 2: Synthesis of I-259

To a solution of I-259-3 (60 mg, 44.61 μmol, 1 eq) in DCM (0.6 mL) was added TFA (307.00 mg, 2.69 mmol, 0.2 mL, 60.36 eq). The mixture was stirred at 25° C. for 2 hrs. The reaction liquid was concentrated by reducing pressure to obtain the crude product. The residue was purified by prep-HPLC (column: CD01-Phenomenex luna C18 150*25*10 um; mobile phase: [water (TFA)-ACN]; gradient: 10%-40% B over 10 min) and lyophilizated to afford I-259 (26.42 mg, 20.99 μmol, 47.05% yield, 100.00% purity, TFA) as a white solid. LCMS: R^{t}=0.282 min, [M+H]⁺=1144.6. SFC: R^{t}=3.136 min, 5.172 min. 1H NMR (400 MHZ, METHANOL-d4) δ=8.38 (s, 1H), 8.35-8.26 (m, 1H), 7.84-7.68 (m, 2H), 7.59-7.40 (m, 2H), 7.38-7.28 (m, 4H), 6.96-6.89 (m, 1H), 5.09-5.01 (m, 1H), 5.00-4.91 (m, 3H), 4.61 (d, J=13.6 Hz, 2H), 4.57-4.42 (m, 1H), 4.40-4.08 (m, 5H), 4.02 (d, J=7.6 Hz, 1H), 3.98-3.62 (m, 10H), 3.57-3.35 (m, 6H), 3.27-2.98 (m, 4H), 2.95-2.83 (m, 1H), 2.83-2.73 (m, 2H), 2.72-2.53 (m, 2H), 2.50-2.25 (m, 2H), 2.24-2.13 (m, 2H), 2.12-1.98 (m, 5H), 1.97-1.82 (m, 6H), 1.77 (s, 2H), 1.73-1.61 (m, 4H), 1.61-1.44 (m, 1H), 1.41-1.19 (m, 3H), 1.18-1.04 (m, 2H), 1.01-0.84 (m, 4H). ¹⁹F NMR (400 MHZ, METHANOL-d4) δ=−77.001.

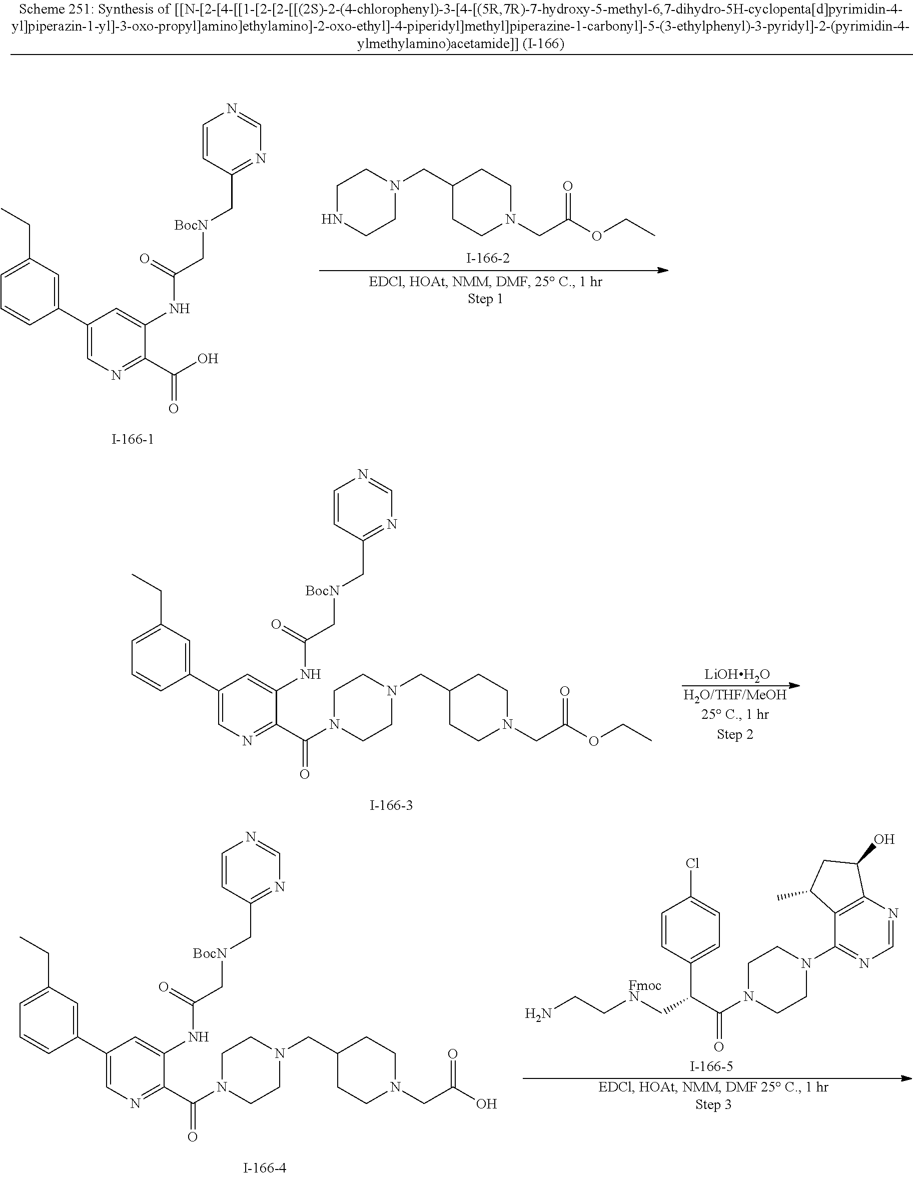

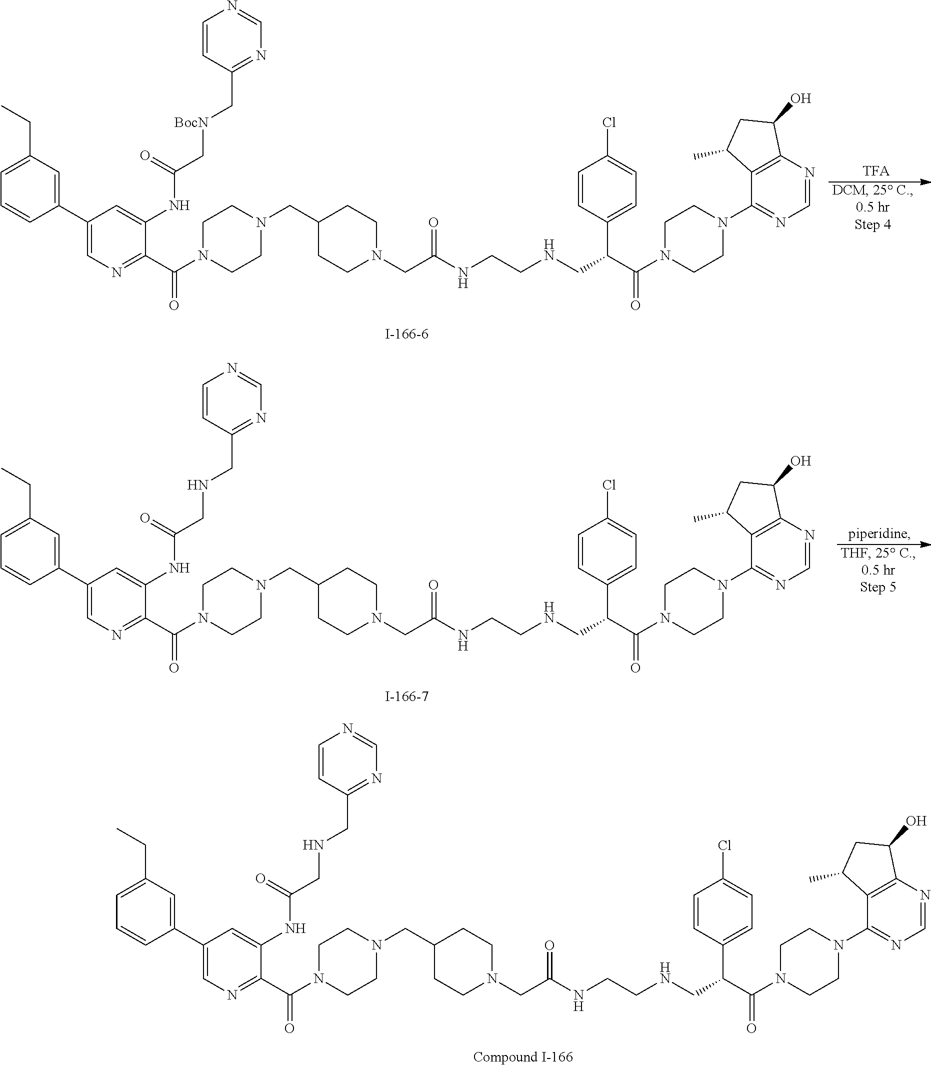

### Step 1: Synthesis of I-166-3.

To a solution of I-166-1 (500 mg, 1.02 mmol, 1 eq) in DMF (5 mL) was added EDCI (585.00 mg, 3.05 mmol, 3 eq), HOAt (138.45 mg, 1.02 mmol, 142.30 μL, 1 eq), NMM (514.44 mg, 5.09 mmol, 559.18 μL, 5 eq) and I-166-2 (373.33 mg, 1.22 mmol, 1.2 eq, HCl). The mixture was stirred at 25° C. for 1 hr. The mixture was concentrated to give crude product. The crude product was purified by reverse-phase column (0.1% FA condition), the eluent was concentrated to remove ACN and lyophilized to give the product. I-166-3 (0.48 g, 591.13 μmol, 58.11% yield, 91.49% purity) was obtained as a yellow solid. LCMS (Method D): R^{t}=0.323 min, [M+H]⁺=743.4.

### Step 2: Synthesis of I-166-4.

To a solution of I-166-3 (400 mg, 538.43 μmol, 1 eq) in H₂O (2 mL), THF (2 mL) and MeOH (2 mL) was added LiOH·H₂O (67.78 mg, 1.62 mmol, 3 eq). The mixture was stirred at 25° C. for 1 hr. The reaction mixture was quenched by H₂O (10 mL), and then extracted with DCM 30 mL (10 mL*3), The combined organic phase was dried over Na₂SO₄, filtered, and concentrated under reduced pressure to give a residue. The crude product was used into the next step without further purification. I-166-4 (340 mg, crude) was obtained as a yellow solid. LCMS (Method D): R^{t}=0.330 min, [M+H]⁺=715.3.

### Step 3: Synthesis of I-166-6.

To a solution of I-166-4 (80 mg, 111.91 μmol, 1 eq) and I-166-5 (96.38 mg, 134.29 μmol, 1.2 eq, HCl) in DMF (1 mL) was added EDCI (64.36 mg, 335.73 μmol, 3 eq), HOAt (15.23 mg, 111.91 μmol, 15.66 μL, 1 eq) and NMM (56.60 mg, 559.56 μmol, 61.52 μL, 5 eq). The mixture was stirred at 25° C. for 1 hr. The mixture was quenched by H₂O 1 mL and extracted with DCM 3 mL (1 mL*3). The combined organic phase was dried over Na₂SO₄, filtered, and concentrated under reduced pressure to give a crude product. The crude product was purified by reverse-phase column (0.1% FA condition) and lyophilized to give the product. I-166-6 (110 mg, 78.32 μmol, 69.99% yield, 98.122% purity) was obtained as a white solid. LCMS (Method D): R^{t}=0.388 min, [M+H]⁺=1378.3.

### Step 4: Synthesis of I-166-7.

To a solution of I-166-6 (90 mg, 65.31 μmol, 1 eq) in DCM (1 mL) was added TFA (307.00 mg, 2.69 mmol, 0.2 mL, 41.23 eq). The mixture was stirred at 25° C. for 0.5 hr. The mixture was concentrated under reduced pressure to give a crude product. The crude product was used into the next step without further purification. I-166-7 (90 mg, crude, TFA) was obtained as a yellow oil. LCMS (Method D): R^{t}=0.400 min, [M/2+H]⁺=639.8.

### Step 5: Synthesis of I-166

To a solution of I-166-7 (90 mg, 64.66 μmol, 1 eq, TFA) in THF (1 mL) was added piperidine (172.44 mg, 2.03 mmol, 0.2 mL). The mixture was stirred at 25° C. for 0.5 hr. The mixture was concentrated under reduced pressure to give a crude product. The crude product was purified by reverse-phase column (0.1% NH₃·H₂O condition), the eluent was concentrated to remove ACN and lyophilized to give the product. I-166 (32.13 mg, 30.01 μmol, 46.41% yield, 98.600% purity) was obtained as a white solid. LCMS (Method D): R^{t}=0.269 min, [M+H]⁺=1055.3. SFC: R^{t}=0.798 min. ¹H NMR (400 MHZ, METHANOL-d4) δ=9.10 (d, J=1.2 Hz, 1H), 8.81 (d, J=1.8 Hz, 1H), 8.77 (d, J=5.2 Hz, 1H), 8.61 (d, J=2.0 Hz, 1H), 8.41 (s, 1H), 7.79 (d, J=5.2 Hz, 1H), 7.54 (s, 1H), 7.51 (d, J=8.0 Hz, 1H), 7.43 (t, J=7.6 Hz, 1H), 7.40-7.35 (m, 2H), 7.35-7.28 (m, 3H), 4.96 (t, J=6.8 Hz, 1H), 4.20-4.16 (m, 1H), 4.01 (s, 2H), 3.91-3.85 (m, 1H), 3.81-3.67 (m, 4H), 3.65-3.57 (m, 2H), 3.57-3.44 (m, 7H), 3.36-3.32 (m, 2H), 3.29-3.23 (m, 2H), 2.97 (s, 2H), 2.84 (d, J=3.2 Hz, 2H), 2.80-2.71 (m, 5H), 2.45 (d, J=4.4 Hz. 4H), 2.23 (d, J=7.2 Hz, 2H). 2.17-2.08 (m, 3H), 2.07-2.01 (m, 1H), 1.76 (d, J=12.0 Hz, 2H), 1.61-1.50 (m, 1H), 1.35-1.25 (m, 5H). 1.10 (d, J=6.8 Hz, 3H).

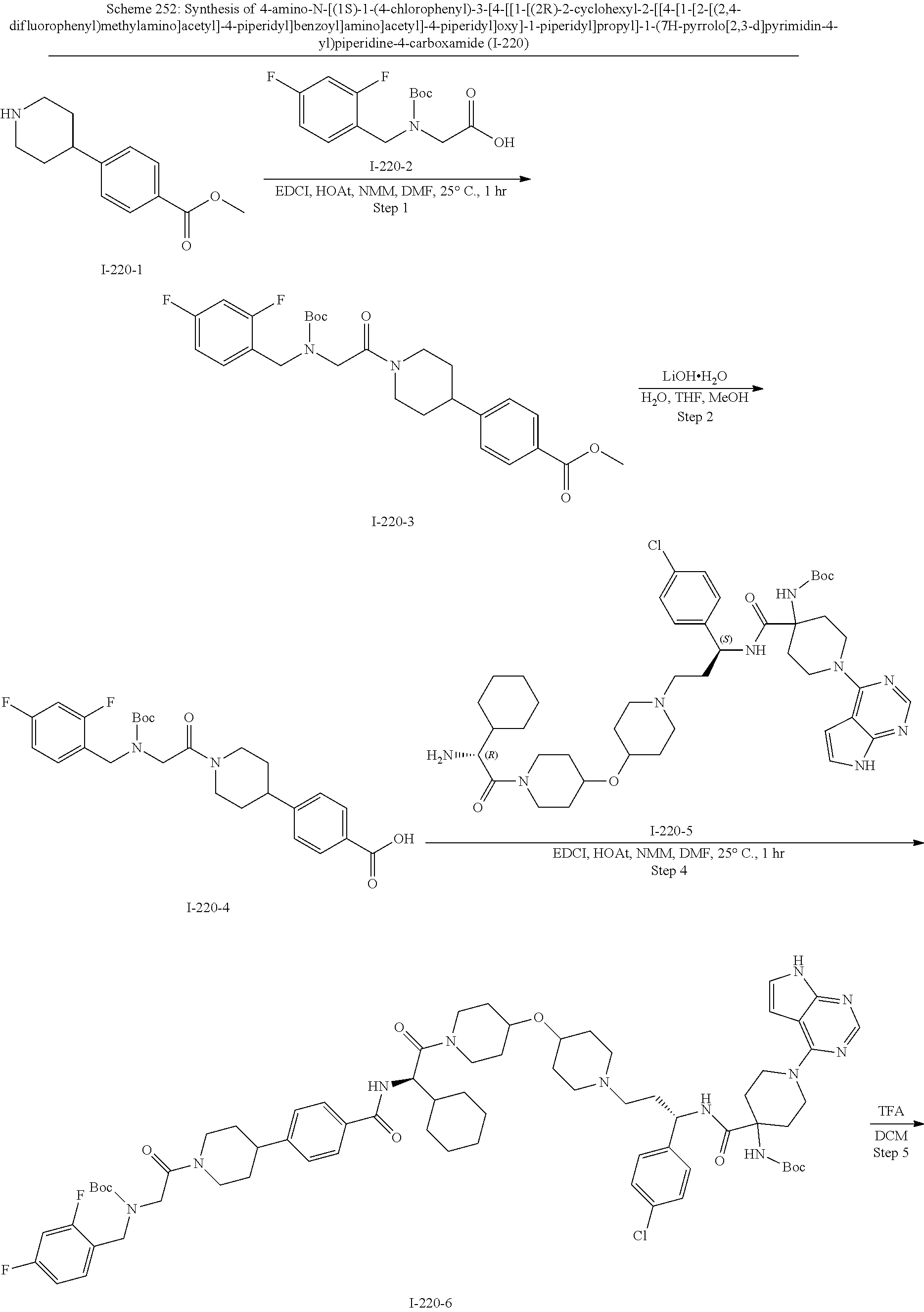

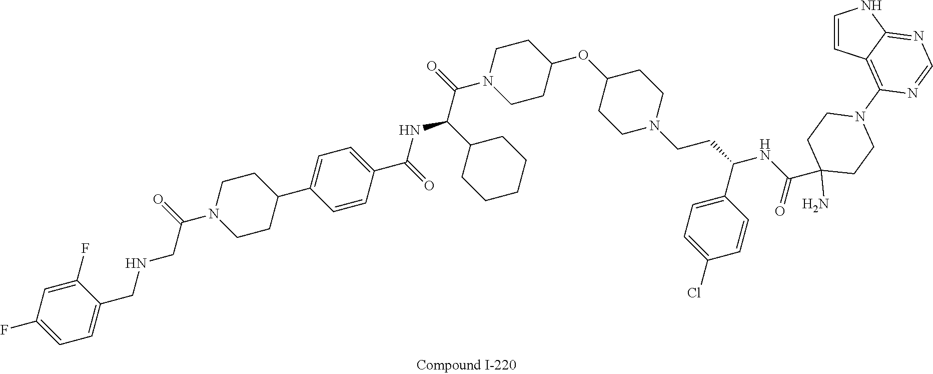 [SMILES: NC1(C(=O)N[C@@H](CCN2CCC(OC3CCN(C(=O)[C@H](NC(=O)c4ccc(C5CCN(C(=O)CNCc6ccc(F)cc6F)CC5)cc4)C4CCCCC4)CC3)CC2)c2ccc(Cl)cc2)CCN(c2ncnc3[nH]ccc23)CC1]

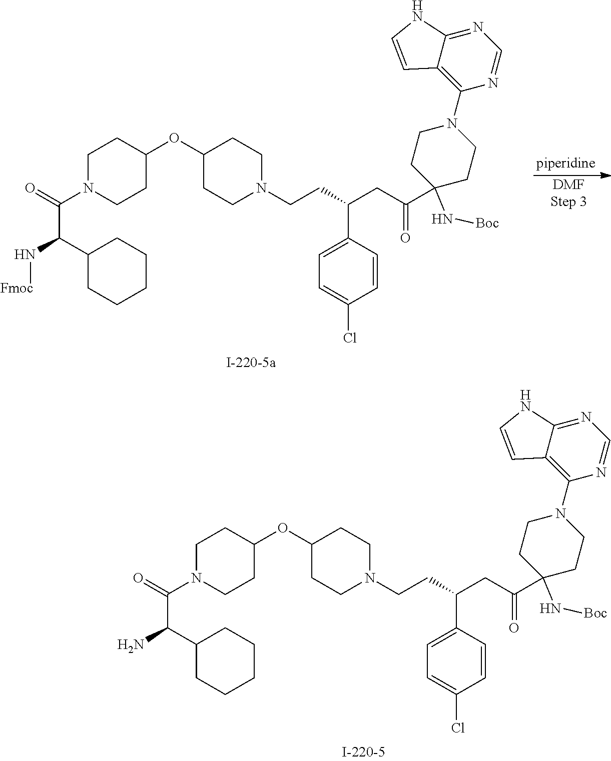

### Step 1: Synthesis of I-220-3.

To a solution of I-220-1 (500 mg, 1.96 mmol, 1 eq, HCl), I-220-2 (589.05 mg, 1.96 mmol, 1 eg) in DMF (5 mL) was added NMM (988.77 mg, 9.78 mmol, 1.07 mL, 5 eq), HOAt (532.22 mg, 3.91 mmol, 546.99 μL, 2 eq) and EDCI (1.87 g, 9.78 mmol, 5 eq). The mixture was stirred at 25° C. for 1 hr. The reaction mixture was diluted with water (5 mL) and extracted with EA (5 mL*3), the combined organic phase was dried by Na₂SO₄, concentrated under reduced pressure to give a residue. The residue was purified by flash silica gel chromatography (ISCO®; 20 g SepaFlash® Silica Flash Column, Eluent of 0˜50% Ethyl acetate/Petroleum ether @ 45 mL/min) and the eluent was concentrated to give product. I-220-3 (915 mg, 1.81 mmol, 92.34% yield, 99.156% purity) was obtained as a white solid. LCMS: R^{t}=0.678 min, [M+H]⁺=503.2.

### Step 2: Synthesis of I-220-4

To a solution of I-220-3 (915 mg, 1.82 mmol, 1 eq) in MeOH (3 mL), THF (3 mL), H₂O (3 mL) was added LiOH·H₂O (229.21 mg, 5.46 mmol, 3 eq). The mixture was stirred at 25° C. for 1 hr. The pH was adjusted to 5 with 1M HCl, then the mixture was extracted with DCM (4 mL*3), the combined organic phase was dried by Na₂SO₄, concentrated under reduced pressure to give a residue. The crude product was used into the next step without further purification. I-220-4 (750 mg, crude) was obtained as a white solid. LCMS: R^{t}=0.624 min, [M+H]⁺=489.1.

### Step 3: Synthesis of I-220-5

To a solution of I-220-5a (100 mg, 94.63 μmol, 1 eq) in DMF (1 mL) was added piperidine (16.12 mg, 189.26 μmol, 18.69 μL, 2 eq) at 25° C., the mixture was stirred at 25° C. for 1 h. The reaction mixture was diluted with water (3 mL) and extracted with DCM (3 mL*3), the combined organic phase was dried by Na₂SO₄ and concentrated under reduced pressure to give a residue. The crude product was used into the next step without further purification. I-220-5 (128 mg, crude) was obtained as a yellow solid. LCMS (Method D): R^{t}=0.285 min, [M+H]⁺=834.6.

### Step 4: Synthesis of I-220-6.

To a solution of I-220-4 (46.83 mg, 95.87 μmol, 1 eq), I-220-5 (80 mg, 95.87 μmol, 1 eq) in DMF (0.8 mL) was added NMM (48.48 mg, 479.34 μmol, 52.70 μL, 5 eq), EDCI (91.89 mg, 479.34 μmol, 5 eq) and HOAt (26.10 mg, 191.73 μmol, 26.82 μL, 2 eq) at 25° C., the mixture was stirred at 25° C. for 30 min. The reaction mixture was diluted with water (3 mL) and extracted with DCM (3 mL*3), the combined organic phase was dried by Na₂SO₄, concentrated under reduced pressure to give a residue. The residue was purified by flash silica gel chromatography (ISCO®; 4 g SepaFlash® Silica Flash Column, Eluent of 0˜50% Methanol/Dichloromethane @20 mL/min) and the eluent was concentrated to give product. I-220-6 (90 mg, 49.51 μmol, 51.64% yield, 71.790% purity) was obtained as a white solid. LCMS (Method D): R^{t}=0.416 min, [M+H]⁺=1304.9.

### Step 5: Synthesis of 1-220

To a solution of I-220-6 (70 mg, 53.64 μmol, 1 eq) in DCM (0.35 mL) was added TFA (537.25 mg, 4.71 mmol, 350.00 μL, 87.84 eq), the mixture was stirred at 25° C. for 30 min. The reaction mixture was concentrated under reduced pressure to give a residue. The crude product was purified by prep-HPLC (column: CD04-Welch Ultimate C18 150*25*7 um; mobile phase: [water (TFA)-ACN]; gradient: 15%-45% B over 18 min), the eluent was concentrated to remove ACN and lyophilized to get product. I-220 (5 mg, 4.10 μmol, 7.65% yield, 100% purity, TFA) was obtained as a white solid. LCMS (Method D): R^{t}=0.319 min, [M+H]⁺=1104.6. ¹H NMR (400 MHZ, METHANOL-d4) δ=8.33 (d, J=2.0 Hz, 1H), 8.22 (d, J=8.0 Hz, 1H), 7.84-7.74 (m, 2H), 7.68-7.58 (m, 1H), 7.41-7.29 (m, 7H), 7.19-7.06 (m, 2H), 6.85 (t, J=3.6 Hz, 1H), 5.04-4.97 (m, 1H), 4.95-4.90 (m, 1H), 4.69-4.58 (m, 3H), 4.33 (s, 2H), 4.25-4.11 (m, 2H), 4.05-3.98 (m, 1H), 3.95-3.88 (m, 1H), 3.86-3.71 (m, 5H), 3.68-3.57 (m, 1H), 3.54-3.47 (m, 1H), 3.41-3.35 (m, 1H), 3.27-3.16 (m, 3H), 3.09-3.01 (m, 1H), 2.99-2.90 (m, 1H), 2.88-2.80 (m, 1H), 2.67-2.51 (m, 2H), 2.41-2.30 (m, 1H), 2.30-2.09 (m, 3H), 2.09-1.90 (m, 7H), 1.85 (d, J=11.2 Hz, 3H), 1.76 (d, J=8.0 Hz, 3H), 1.72-1.58 (m, 5H), 1.53 (s, 1H), 1.42-0.99 (m, 6H). ¹⁹F NMR (377 MHz, METHANOL-d4), δ=−76.99 (s, 20F), −108.22-−108.55 (m, 1F), −112.92 (s, 1F).

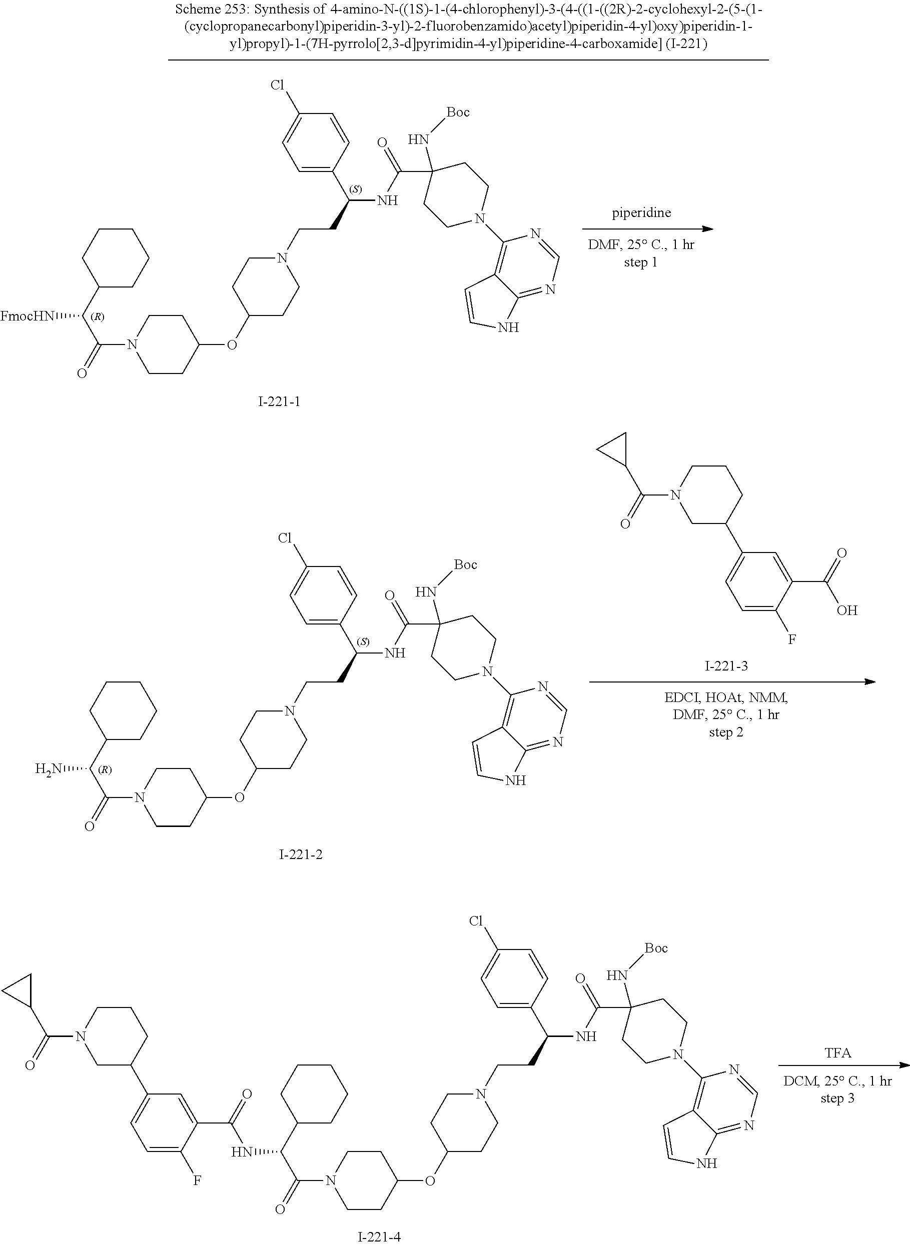

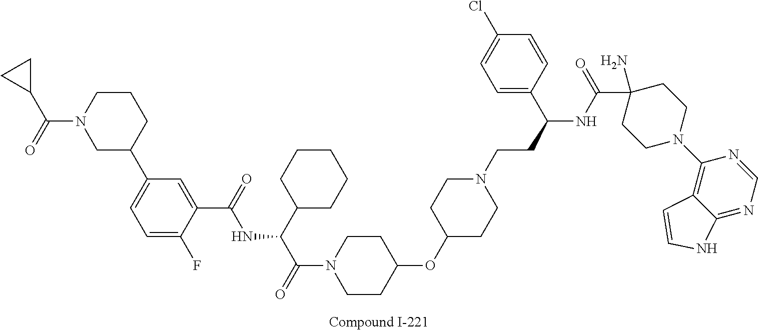 [SMILES: NC1(C(=O)N[C@@H](CCN2CCC(OC3CCN(C(=O)[C@H](NC(=O)c4cc(C5CCCN(C(=O)C6CC6)C5)ccc4F)C4CCCCC4)CC3)CC2)c2ccc(Cl)cc2)CCN(c2ncnc3[nH]ccc23)CC1]

### Step 1: Synthesis of I-221-2

To a solution of I-221-1 (100 mg, 94.63 μmol, 1 eq) in DMF (1 mL) was added piperidine (16.12 mg, 189.26 μmol, 18.69 μL, 2 eq). The mixture was stirred at 25° C. for 1 hr. The reaction mixture was added H₂O (2 mL) and then extracted with DCM (2 mL*3), the combined organic phase was washed with dried by Na₂SO₄, filtered, and concentrated. The crude product was used into next step directly. I-221-2 (80 mg, crude) was obtained as a white solid. LCMS (Method D): R^{t}=0.290 min, [M+H]⁺=330.9.

### Step 2: Synthesis of I-221-4

To a solution of I-221-2 (70 mg, 83.88 μmol, 1 eq), I-221-3 (24.44 mg, 83.88 μmol, 1 eq) in DMF (0.7 mL) was added EDCI (32.16 mg, 167.77 μmol, 2 eq) and HOAt (11.42 mg, 83.88 μmol, 11.73 μL, 1 eq) and NMM (42.42 mg, 419.42 μmol, 46.11 μL, 5 eq). The mixture was stirred at 25° C. for 1 hr. The reaction mixture was added H₂O (1 mL) and then extracted with DCM (1 mL*3), the combined organic phase was washed with dried by Na₂SO₄, filtered, and concentrated. The residue was purified by flash silica gel chromatography (ISCO®; 12 g SepaFlash® Silica Flash Column, Eluent of 0˜100% Ethyl acetate/Petroleum ether gradient (@12 mL/min) and the eluent was concentrated to give product. I-221-4 (90 mg, 81.24 μmol, 96.85% yield, N/A purity) was obtained as a white solid. LCMS (Method D): R^{t}=0.390 min, [M+H]⁺=1107.7. SFC: R^{t}=6.383 min, 8.443 min.

### Step 3: Synthesis of I-221.

To a solution of I-221-4 (80 mg, 72.22 μmol, 1 eq) in DCM (0.9 mL) was added TFA (8.23 mg, 72.22 μmol, 0.3 mL, 1 eq). The mixture was stirred at 25° C. for 1 hr. The reaction mixture was concentrated under reduced pressure to give a residue. The residue was purified by prep-HPLC (column: CD04-Welch Ultimate C18 150*25*7 um; mobile phase: [water (TFA)-ACN]; gradient: 20%-50% B over 11 min) the eluent was concentrated to remove ACN and lyophilized to get product. I-221 (20 mg, 17.68 μmol, 24.48% yield, 99.133% purity, TFA) was obtained as a white solid. LCMS (Method D): R^{t}=0.408 min, [M+H]⁺=1007.7. SFC: R^{t}=6.384 min, 7.767 min. ¹H NMR (400 MHZ, METHANOL-d4)=8.34 (d, J=1.2 Hz, 1H), 7.74-7.58 (m, 1H), 7.49 (d, J=18.0 Hz, 1H), 7.42-7.27 (m, 5H), 7.26-7.11 (m, 1H), 6.86 (t, J=3.6 Hz, 1H), 5.05-4.93 (m, 2H), 4.63 (s, 2H), 4.54 (d, J=9.2 Hz, 1H), 4.36 (s, 1H), 4.02 (s, 1H), 3.96-3.86 (m, 1H), 3.80 (s, 4H), 3.65-3.46 (m, 2H), 3.37 (d, J=2.4 Hz, 1H), 3.30-3.16 (m, 4H), 3.16-2.92 (m, 2H), 2.89-2.68 (m, 2H), 2.67-2.52 (m, 2H), 2.45-2.30 (m, 1H), 2.29-2.08 (m, 3H), 2.07-1.89 (m, 7H), 1.89-1.72 (m, 7H), 1.72-1.58 (m, 4H), 1.58-1.49 (m, 1H), 1.35-1.19 (m, 3H), 1.18-1.07 (m, 2H), 0.93-0.72 (m, 4H). ¹⁹F NMR 19 (377 MHz, METHANOL-d4) δ=−77.02.

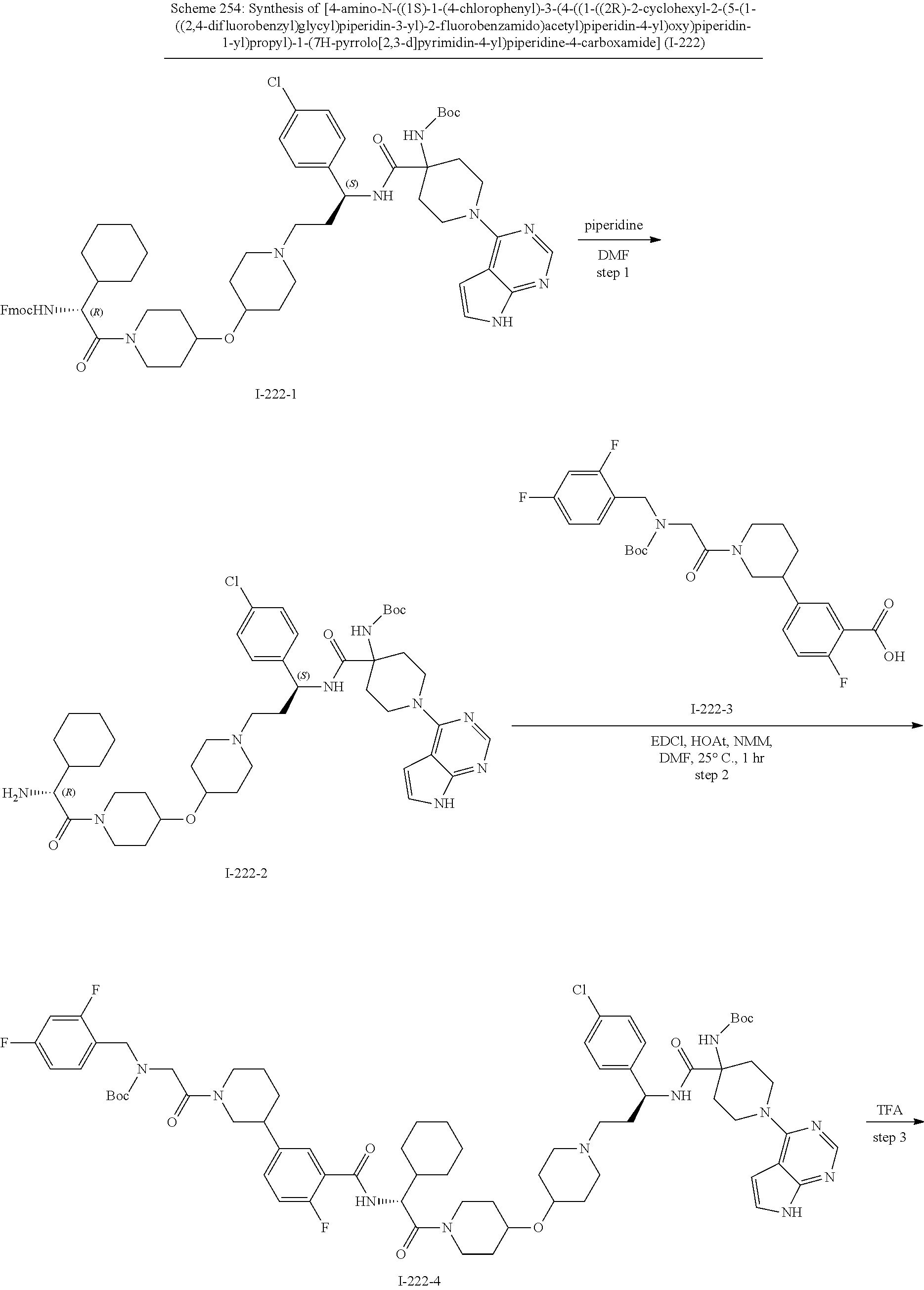

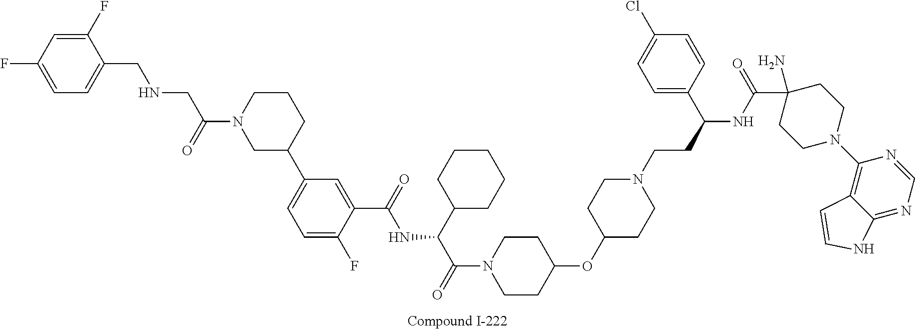 [SMILES: NC1(C(=O)N[C@@H](CCN2CCC(OC3CCN(C(=O)[C@H](NC(=O)c4cc(C5CCCN(C(=O)CNCc6ccc(F)cc6F)C5)ccc4F)C4CCCCC4)CC3)CC2)c2ccc(Cl)cc2)CCN(c2ncnc3[nH]ccc23)CC1]

### Step 1: Synthesis of I-222-2

To a solution of I-222-1 (80 mg, 75.71 μmol, 1 eq) in DMF (1 mL) was added piperidine (19.34 mg, 227.12 μmol, 22.43 μL, 3 eq). The mixture was stirred at 25° C. for 0.5 hr. The reaction mixture was diluted with H₂O 4 mL and extracted with DCM 15 mL (5 mL*3). The combined organic layers were washed with NaCl (aq) 2 mL, dried over Na₂SO₄, filtered, and concentrated under reduced pressure to give I-222-2 (60 mg, crude) was obtained as a white solid. LCMS: R^{t}=0.335 min, [M+H]⁺=834.6.

### Step 2: Synthesis of I-222-4

To a solution of I-222-2 (60 mg, 71.90 μmol, 1 eq), I-222-3 (36.42 mg, 71.90 μmol, 1 eq) in DMF (1 mL) was added EDCI (41.35 mg, 215.70 μmol, 3 eq) and HOAt (9.79 mg, 71.90 μmol, 10.06μ, 1 eq), NMM (36.36 mg, 359.50 μmol, 39.52 μL, 5 eq). The mixture was stirred at 25° C. for 0.5 hr. The reaction mixture was diluted with H₂O 2 mL and extracted with DCM 8 mL (4 mL*2). The combined organic layers were washed with NaCl (aq) 2 mL, dried over Na₂SO₄, filtered, and concentrated under reduced pressure to give a residue. The residue was purified by flash silica gel chromatography (ISCO®; 12 g SepaFlash® Silica Flash Column, Eluent of 0˜100% Ethyl acetate/Petroleum ether gradient @ 20 mL/min) was concentrated under reduced pressure to give I-222-4 (70 mg, 52.91 μmol, 73.59% yield) as a white solid. LCMS: R^{t}=0.415 min, [M+H]⁺=1322.8. SFC: R^{t}=3.687 min, 3.954 min.

### Step 3: Synthesis of I-222

To a solution of I-222-4 (60 mg, 45.35 μmol, 1 eq) in DCM (0.6 mL) was added TFA (184.20 mg, 1.62 mmol, 120.00 μL, 35.62 eq). The mixture was stirred at 25° C. for 0.5 hr. The reaction mixture was concentrated under reduced pressure to give a residue. The residue was purified by prep-HPLC (column: CD02-Waters Xbidge BEH C18 150*25*10 um; mobile phase: [water (NH₄HCO₃)-ACN]; gradient: 50%-80% B over 11 min) the eluent was concentrated to remove ACN and lyophilized to get I-222 (10.38 mg, 9.25 μmol, 20.39% yield, 100% purity) as a white solid. LCMS: R^{t}=0.332 min, [M+H]⁺=1122.3. SFC: R^{t}=5.528 min, 6.108 min. ¹H NMR (400 MHZ, METHANOL-d4) δ=8.12 (d, J=2.0 Hz, 1H), 7.65 (d, J=6.0 Hz, 1H), 7.50-7.41 (m, 2H), 7.32 (d, J=2.0 Hz, 4H), 7.23-7.15 (m, 1H), 7.13-7.11 (m, 1H), 6.99-6.89 (m, 2H), 6.63-6.61 (m, 1H), 5.00-4.97 (m, 1H), 4.93-4.88 (m, 1H), 4.57-4.43 (m, 3H), 4.08-3.95 (m, 1H), 3.85 (s, 1H), 3.81 (d, J=7.6 Hz, 3H), 3.77-3.71 (m, 1H), 3.69-3.60 (m, 2H), 3.58-3.48 (m, 3H), 3.48-3.41 (m, 1H), 3.29-3.20 (m, 1H), 3.14-3.00 (m, 1H), 2.87-2.65 (m, 4H), 2.37 (s, 2H), 2.31-2.10 (m, 4H), 2.07-1.97 (m, 3H), 1.96-1.73 (m, 11H), 1.68 (d, J=9.6 Hz, 2H), 1.61 (d, J=11.6 Hz, 5H), 1.52-1.42 (m, 1H), 1.35-1.20 (m, 3H), 1.19-1.08 (m, 2H). ¹⁹F NMR (376 MHz, METHANOL-d4) δ=−116.00-−116.44 (m, 1F), −118.46 (br d, J=19.8 Hz, 1F), −118.62-−118.81 (m, 1F).

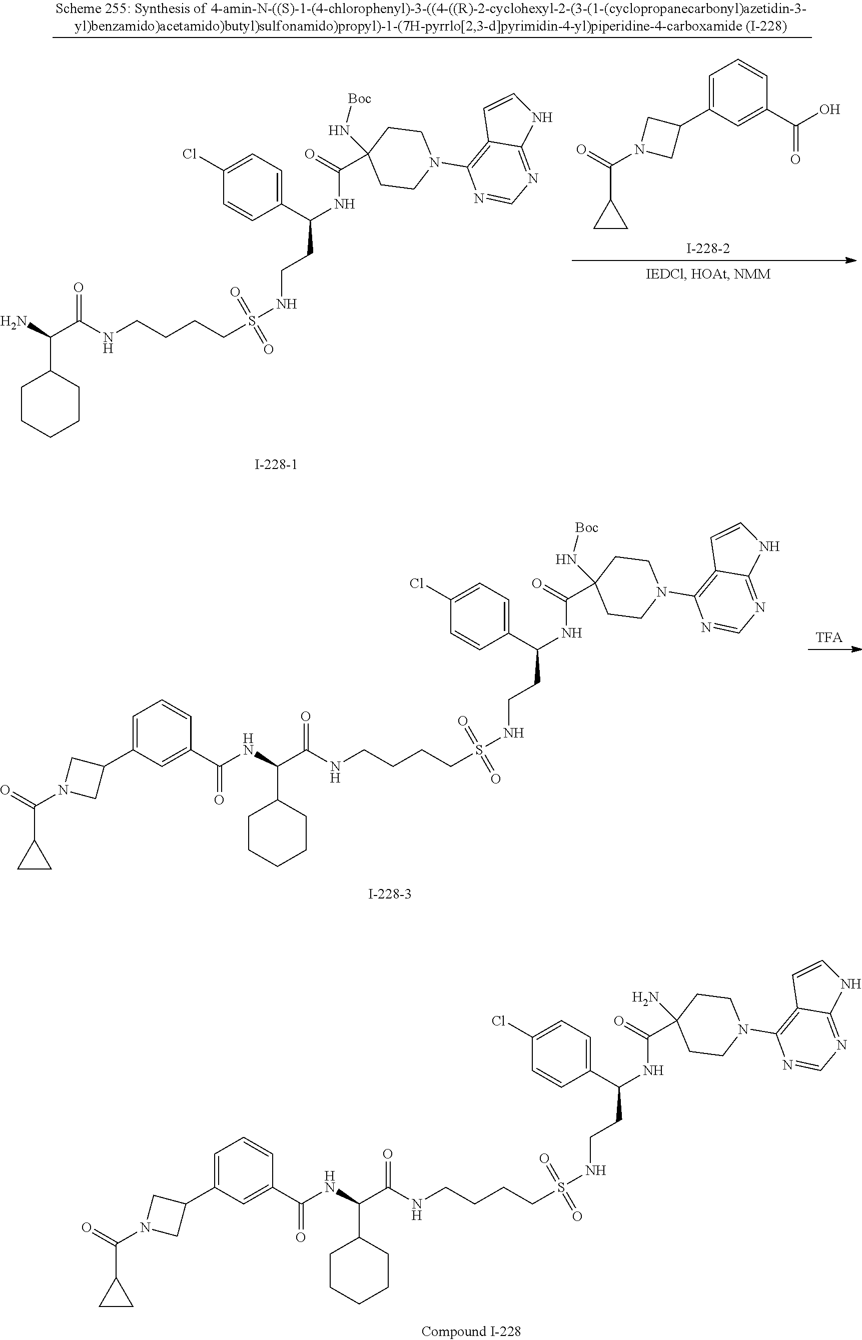 [SMILES: CC(C)(C)OC(=O)NC1(C(=O)N[C@@H](CCNS(=O)(=O)CCCCNC(=O)[C@H](N)C2CCCCC2)c2ccc(Cl)cc2)CCN(c2ncnc3[nH]ccc23)CC1 ; O=C(O)c1cccc(C2CN(C(=O)C3CC3)C2)c1 ; CC(C)(C)OC(=O)NC1(C(=O)N[C@@H](CCNS(=O)(=O)CCCCNC(=O)[C@H](NC(=O)c2cccc(C3CN(C(=O)C4CC4)C3)c2)C2CCCCC2)c2ccc(Cl)cc2)CCN(c2ncnc3[nH]ccc23)CC1 ; NC1(C(=O)N[C@@H](CCNS(=O)(=O)CCCCNC(=O)[C@H](NC(=O)c2cccc(C3CN(C(=O)C4CC4)C3)c2)C2CCCCC2)c2ccc(Cl)cc2)CCN(c2ncnc3[nH]ccc23)CC1]

### Step 1: Synthesis of I-228-3

A mixture of I-228-1 (60 mg, 74.77 μmol, 1 eq), I-228-2 (18.34 mg, 74.77 μmol, 1 eq) and HOAt (10.18 mg, 74.77 μmol, 10.46 μL, 1 eq) in DMF (1 mL) was added EDCI (28.67 mg, 149.55 μmol, 2 eq) and NMM (37.82 mg, 373.87 μmol, 41.11 μL, 5 eq). The mixture was stirred at 25° C. for 1 hrs. The mixture was poured into water (6 mL) and filtered. The filter cake was concentrated under vacuum. The residue was purified by Pre-HPLC (column: Waters Xbridge 150*25 mm*5 um; mobile phase: [water (NH₄HCO₃)-ACN]; gradient: 33%-63% B over 10 min) and concentrated under vacuum. I-228-3 (50 mg, 48.56 μmol, 64.94% yield) was obtained as white solid SFC(_F3). LCMS: R^{t}=1.886 min, [M−H]⁺=1029.7. SFC: R^{t}=1.084 min. F3.

### Step 2: Synthesis of I-228

To a solution of I-228-3 (50 mg, 48.56 μmol, 1 eq) in DCM (1 mL) was added TFA (153.50 mg, 1.35 mmol, 0.1 mL, 27.72 eq). The mixture was stirred at 25° C. for 2 hr. The mixture was adjusted to pH=8 with NH₃·H₂O and concentrated under vacuum. The residue was purified by Pre-HPLC (column: Waters Xbridge 150*25 mm*5 um; mobile phase: [water (NH4HCO3)-ACN]; gradient: 32%-62% B over 9 min) and dried by lyophilization. I-228 (27.5 mg, 26.22 μmol, 54.00% yield, 99.515% purity, TFA) was obtained as white solid. LCMS: R^{t}=0.458 min, [M+H]⁺=929.4. SFC: R^{t}=1.610 min. 1404. ¹H NMR (400 MHZ, METHANOL-d4) δ=8.13 (s, 1H), 7.86 (s, 1H), 7.74 (d, J=7.6 Hz, 1H), 7.55-7.50 (m, 1H), 7.49-7.42 (m, 1H), 7.31 (s, 4H), 7.13 (d, J=3.6 Hz, 1H), 6.62 (d, J=3.6 Hz, 1H), 5.01-5.00 (m, 1H), 4.78-4.69 (m, 1H), 4.56-4.45 (m, 2H), 4.44-4.33 (m, 2H), 4.29 (d, J=8.4 Hz, 1H), 4.06-3.92 (m, 2H), 3.69-3.56 (m, 2H), 3.24-3.15 (m, 1H), 3.10-3.01 (m, 4H), 2.31-2.15 (m, 2H), 2.05-1.92 (m, 2H), 1.90-1.73 (m, 6H), 1.71-1.55 (m, 7H), 1.36-1.00 (m, 6H), 0.91-0.86 (m, 2H), 0.85-0.80 (m, 2H). ¹⁹F NMR (377 MHz, METHANOL-d4) δ=−76.94 (s, 1F).

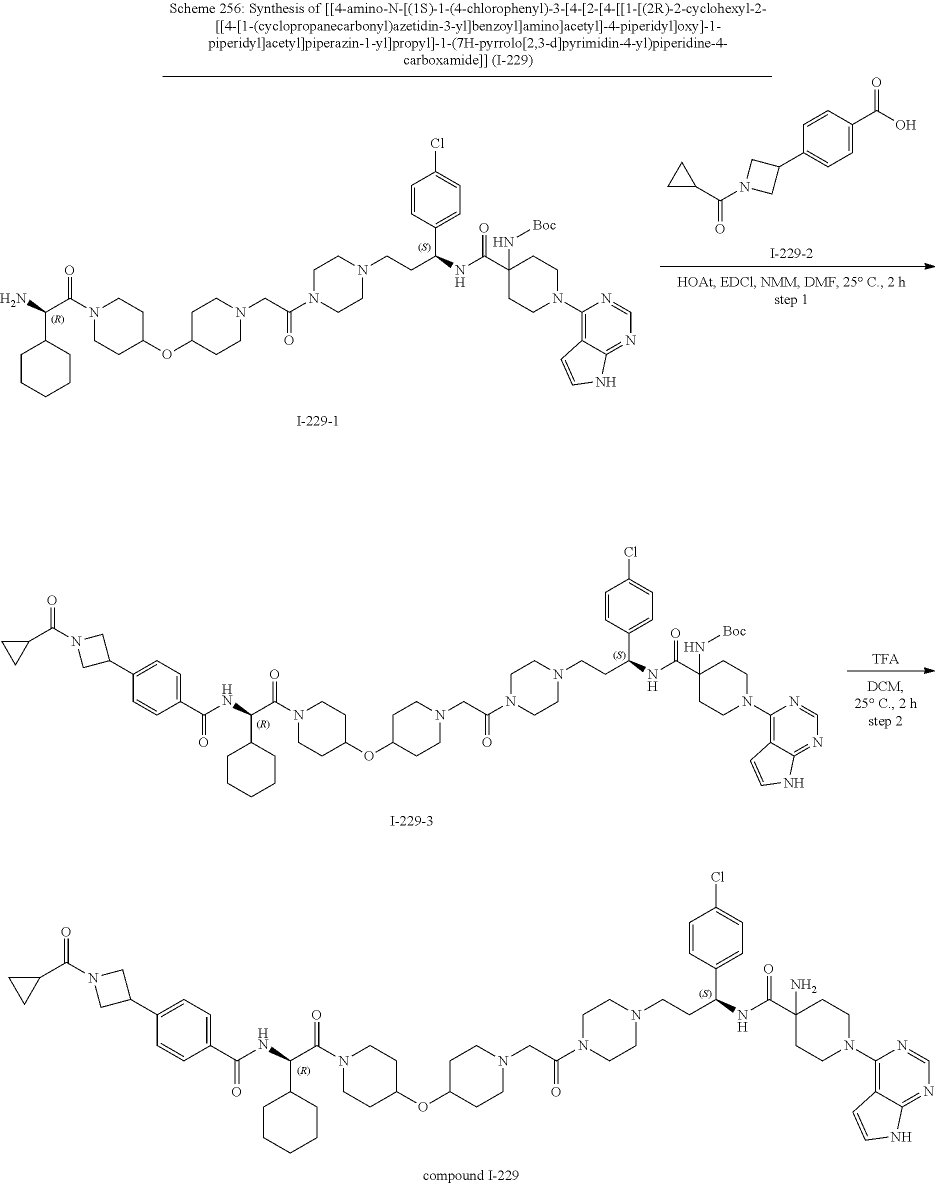

### Step 1: Synthesis of I-229-3.

To a solution of I-229-2 (38.30 mg, 156.15 μmol, 1.5 eq) in DMF (1 mL) was added EDCI (59.87 mg, 312.29 μmol, 3 eq), HOAt (14.17 mg, 104.10 μmol, 14.56 μL, 1 eq), NMM (52.65 mg, 520.48 μmol, 57.22 μL, 5 eq) and I-229-1 (100 mg, 104.10 μmol, 1 eq). The mixture was stirred at 25° C. for 1 hr. The reaction mixture was poured into water (5 mL) and extracted with EA (3 mL*3). The combined organic layers were dried over Na₂SO₄, filtered, and concentrated under reduced pressure to give a residue. The residue was purified by flash silica gel chromatography (ISCO®; 4 g SepaFlash® Silica Flash Column, Eluent of 0˜100% DCM/MeOH @ 18 mL/min, DCM/MeOH=10:1, Rf=0.2) and the eluent was concentrated under reduced pressure to give I-229-3 (110 mg, 92.60 μmol, 88.96% yield), which was obtained as a yellow solid. LCMS (Method D): R^{t}=0.348 min, [M+H]⁺=1187.4.

### Step 2: Synthesis of I-229

To a solution of I-229-3 (100 mg, 84.18 μmol, 1 eq) in DCM (1 mL) was added TFA (460.50 mg, 4.04 mmol, 300.00 μL, 47.98 eq). The mixture was stirred at 25° C. for 0.5 hr. The reaction mixture was concentrated under reduced pressure to give a residue. The residue was purified by reverse phase column (0.1% NH₄HCO₃ condition) and the eluent was lyophilized to give I-229 (34.39 mg, 31.61 μmol, 37.56% yield, 100% purity), which was obtained as a white solid. LCMS (Method D): R^{t}=0.311 min, [M+H]⁺=1087.3. SFC: R^{t}=1.523. ¹H NMR (400 MHZ, METHANOL-d4) δ=8.12 (d, J=1.6 Hz, 1H), 7.85 (d, J=8.4 Hz, 2H), 7.50-7.47 (m, 2H), 7.33 (s, 4H), 7.13-7.11 (m, 1H), 6.63-6.61 (m, 1H), 5.01-4.95 (m, 1H), 4.93 (d, J=8.4 Hz, 1H), 4.80-4.74 (m, 1H), 4.56-4.46 (m, 2H), 4.44-4.32 (m, 2H), 4.07-3.97 (m, 3H), 3.93-3.72 (m, 2H), 3.71-3.50 (m, 8H), 3.50-3.43 (m, 1H), 3.28-3.19 (m, 1H), 2.90-2.77 (m, 2H), 2.55-2.43 (m, 3H), 2.42-2.27 (m, 5H), 2.09 (s, 3H), 2.04-1.95 (m, 2H), 1.94-1.81 (m, 6H), 1.81-1.74 (m, 2H), 1.73-1.50 (m, 9H), 1.35-1.21 (m, 3H), 1.16-1.05 (m, 2H), 0.93-0.87 (m, 2H), 0.87-0.81 (m, 2H). ¹⁹F NMR (400 MHZ, METHANOL-d4) δ=−76.941.

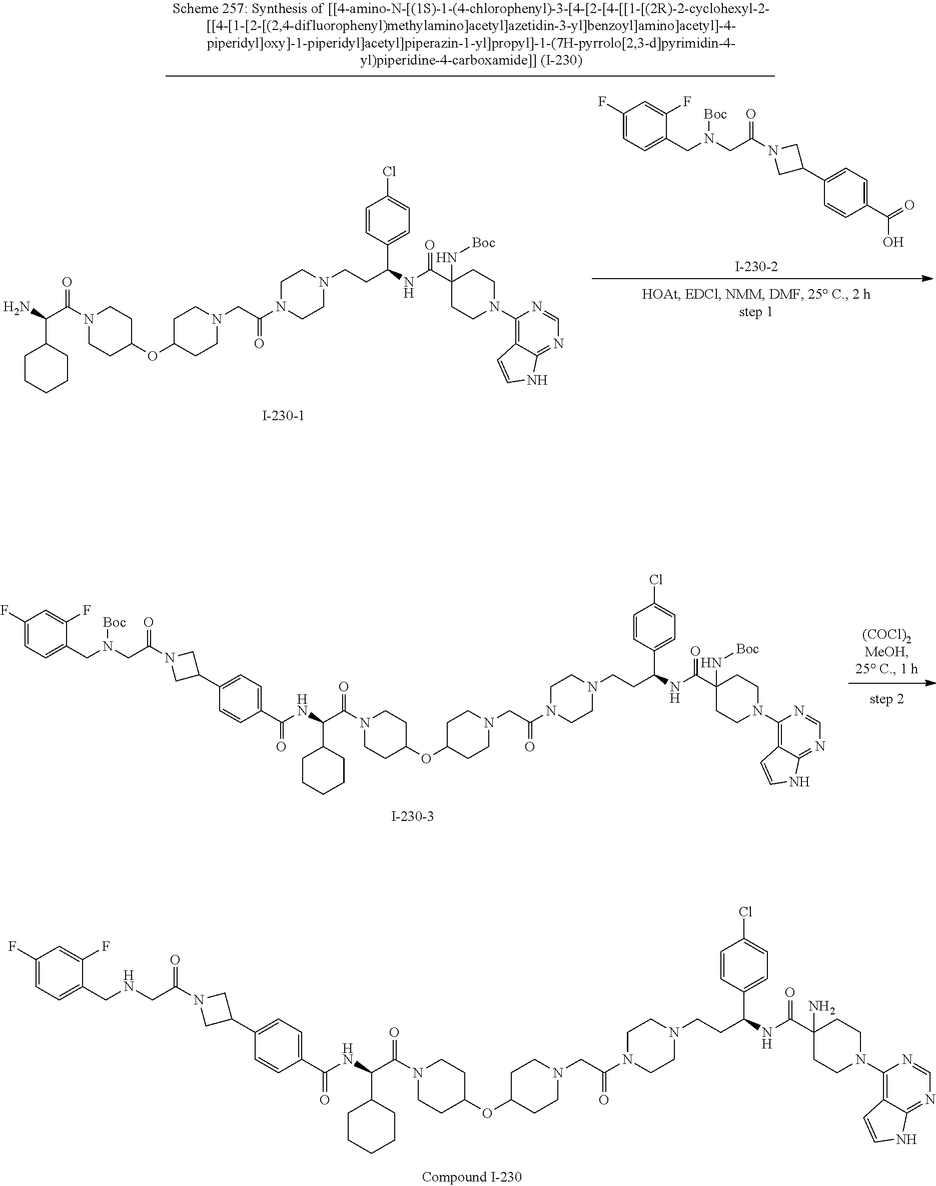

### Step 1: Synthesis of I-230-3

To a solution of I-230-2 (71.90 mg, 156.15 μmol, 1.5 eq) in DMF (1 mL) was added EDCI (99.78 mg, 520.48 μmol, 5 eq), NMM (105.29 mg, 1.04 mmol, 114.45 μL, 10 eq) and HOAt (28.34 mg, 208.19 μmol, 29.12 μL, 2 eq) followed by I-230-1 (100 mg, 104.10 μmol, 1 eq). The mixture was stirred at 25° C. for 1 hr. The residue was diluted with H₂O 5 mL and extracted with EA 15 mL (5 mL*3), dried over Na₂SO₄, filtered, and concentrated under reduced pressure to give a residue. The residue was purified by flash silica gel chromatography (ISCO®; 4 g SepaFlash® Silica Flash Column, Eluent of 0˜100% Dichloromethane/Methanol @ 60 mL/min). The eluent was concentrated under reduced pressure to give product. I-230-3 (130 mg, 87.09 μmol, 83.67% yield, 94% purity) was obtained as a yellow solid. LCMS (Method D): R^{t}=0.388 min, [M+H]⁺=1403.1.

### Step 2: Synthesis of I-230

To a solution of I-230-3 (130 mg, 92.65 μmol, 1 eq) in MeOH (1.3 mL) was added (COCl)₂ (35.28 mg, 277.96 μmol, 24.33 μL, 3 eq). The mixture was stirred at 25° C. for 10 hrs. The residue was concentrated under reduced pressure to give a residue. The residue was purified by prep-HPLC (column: CD02-Waters Xbidge BEH C18 150*25*10 um; mobile phase: [water (NH3H₂O)-ACN]; gradient: 40%-60% B over 10 min). The eluent was lyophilization to give product. I-230 (16 mg, 12.94 μmol, 13.97% yield, 97.298% purity) was obtained as a white solid. LCMS: R^{t}=0.685 min, [M+H]⁺=1202.6. LCMS: R^{t}=0.492 min, [M+H]⁺=1022.4. SFC: R^{t}=1.331 min. ¹H NMR (400 MHz, METHANOL-d4) δ=8.12 (s, 1H), 7.85 (d, J=8.0 Hz, 2H), 7.47-7.45 (m, 2H), 7.33 (s, 5H), 7.14-7.10 (m, 1H), 6.94 (t, J=8.8 Hz, 2H), 6.63-6.60 (m, 1H), 5.00-4.92 (m, 2H), 4.64-4.57 (m, 1H), 4.54-4.43 (m, 3H), 4.27-4.19 (m, 1H), 4.07-3.99 (m, 2H), 3.82 (s, 2H), 3.68-3.60 (m, 7H), 3.55-3.44 (m, 2H), 3.19-3.13 (m, 3H), 2.83-2.71 (m, 3H), 2.51-2.33 (m, 7H), 2.28-2.13 (m, 5H), 2.08-1.93 (m, 3H), 1.91-1.84 (m, 5H), 1.80-1.67 (m, 4H), 1.64-1.54 (m, 6H), 1.35-1.06 (m, 6H). ¹⁹F NMR (377 MHz, METHANOL-d4) δ=−113.37-−116.41.

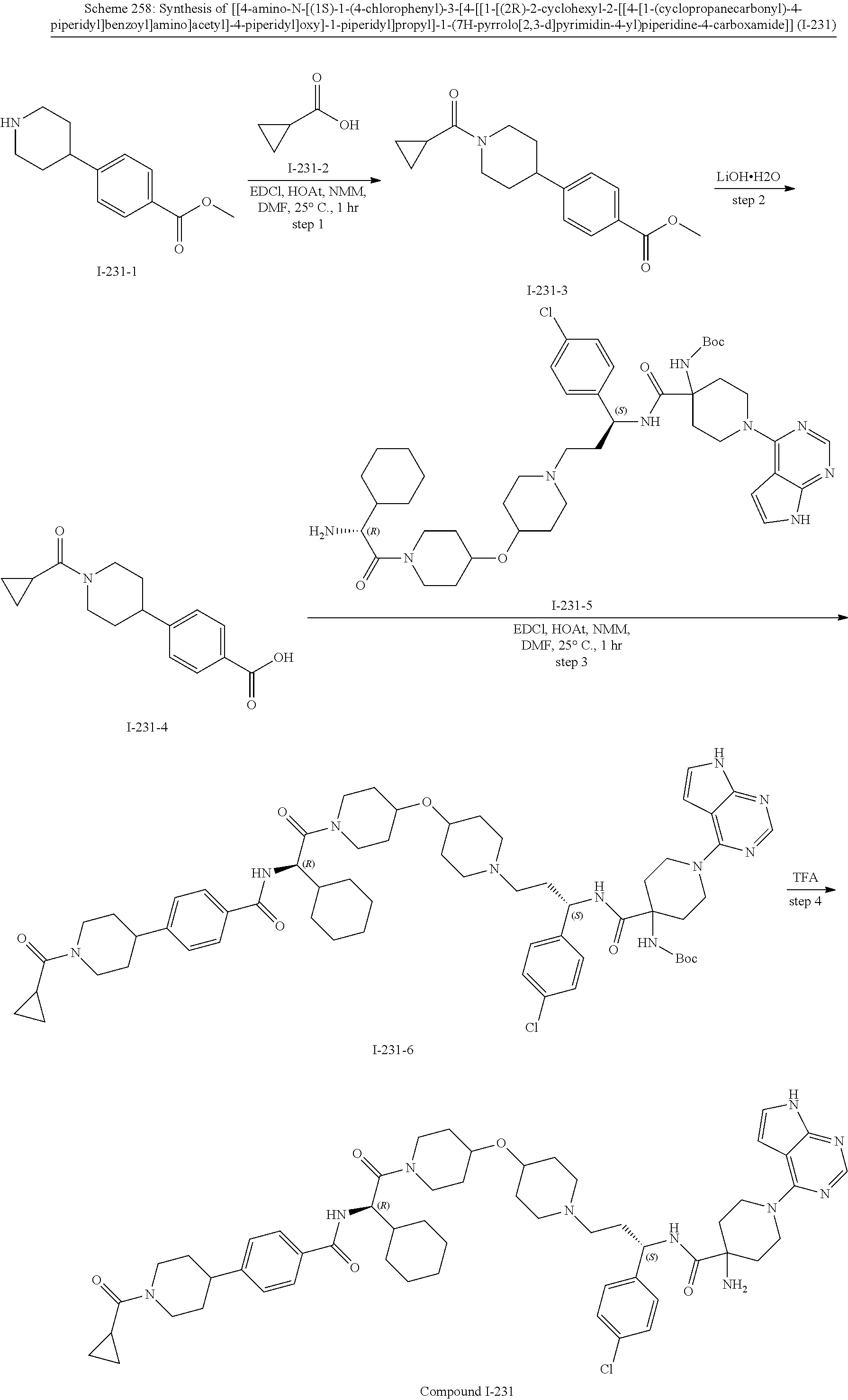

### Step 1: Synthesis of I-231-3

To a solution of I-231-1 (500 mg, 1.96 mmol, 1 eq, HCl), I-231-2 (168.31 mg, 1.96 mmol, 154.70 μL, 1 eq) in DMF (5 mL) was added NMM (988.77 mg, 9.78 mmol, 1.07 mL, 5 eq), HOAt (532.22 mg, 3.91 mmol, 546.99 μL, 2 eq) and EDCI (1.87 g. 9.78 mmol, 5 eq). The mixture was stirred at 25° C. for 1 hr. The reaction mixture was washed with water (5 mL) and extracted with EA (5 mL*3), the combined organic phase was dried by Na₂SO₄, concentrated under reduced pressure to give a residue. The residue was purified by flash silica gel chromatography (ISCO®; 20 g SepaFlash® Silica Flash Column, Eluent of 0˜50% Ethyl acetate/Petroleum ether @50 mL/min) and the eluent was concentrated to give product. I-231-3 (480 mg, 1.58 mmol, 81.01% yield, 94.816% purity) was obtained as a white solid. LCMS: R^{t}=0.515 min, [M+H]⁺=288.2.

### Step 2: Synthesis of I-231-4

To a solution of I-231-3 (380 mg, 1.32 mmol, 1 eq) in MeOH (1.2 mL), THF (1.2 mL), H₂O (1.2 mL) was added LiOH·H₂O (166.48 mg, 3.97 mmol, 3 eq). The mixture was stirred at 25° C. for 1 hr. The mixture was adjusted to a pH to 5 with 1M HCl, then the reaction mixture was extracted with DCM (4 mL*3), the combined organic phase was dried by Na₂SO₄, concentrated under reduced pressure to give product. I-231-4 (350 mg, crude) was obtained as a white solid. LCMS: R^{t}=0.448 min, [M+H]⁺=274.2.

### Step 3: Synthesis of I-231-6

To a solution of I-231-5 (29.48 mg, 107.85 μmol, 1 eq), I-231-4 (90 mg, 107.85 μmol, 1 eq) in DMF (0.9 mL) was added NMM (54.54 mg, 539.25 μmol, 59.29μ, 5 eq), EDCI (103.38 mg, 539.25μmol, 5 eq) and HOAt (29.36 mg, 215.70 μmol, 30.17 μL, 2 eq) at 25° C., the mixture was stirred at 25° C. for 30 mins. The reaction mixture was quenched with water (1 mL) and extracted with DCM (1 mL*3), the combined organic phase was dried by Na₂SO₄, concentrated under reduced pressure to give a residue. The residue was purified by flash silica gel chromatography (ISCO®; 4 g SepaFlash® Silica Flash Column, Eluent of 0˜50% Methanol/Dichloromethane @20 mL/min) and the eluent was concentrated to give product. I-231-6 (100 mg, 91.35 μmol, 84.70% yield, 99.550% purity) was obtained as a white solid. LCMS (Method D): R^{t}=0.365 min, [M+H]⁺=1089.7.

### Step 4: Synthesis of I-231

To a solution of I-231-6 (80 mg, 73.41 μmol, 1 eq) in DCM (0.8 mL) was added TFA (614.00 mg, 5.39 mmol, 400.00 μL, 73.36 eq), the mixture was stirred at 25° C. for 30 mins. The reaction mixture was filtered, and concentrated under reduced pressure to give a residue. The crude was purified by prep-HPLC (column: CD04-Welch Ultimate C18 150*25*7 um; mobile phase: [water (TFA)-ACN]; gradient: 18%-48% B over 18 min) the eluent was concentrated to remove ACN and lyophilized to get products. I-231 (18 mg, 16.15 μmol, 21.99% yield, 98.998% purity, TFA) was obtained as a white solid. LCMS (Method D): R^{t}=0.350 min, [M+H]⁺=989.4. ¹H NMR (400 MHZ, METHANOL-d4) δ=8.37 (s, 1H), 7.78 (d, J=8.4 Hz, 2H), 7.42-7.31 (m, 7H), 6.92 (t, J=3.2 Hz, 1H), 5.04-4.97 (m, 1H), 4.91 (d, J=8.8 Hz, 1H), 4.70-4.57 (m, 3H), 4.48 (d, J=13.6 Hz, 1H), 4.02 (d, J=6.4 Hz, 1H), 3.95-3.70 (m, 5H), 3.65-3.47 (m, 2H), 3.36 (s, 1H), 3.29-3.17 (m, 3H), 3.16-2.98 (m, 2H), 2.97-2.88 (m, 1H), 2.81-2.54 (m, 3H), 2.33 (d, J=10.0 Hz, 1H), 2.30-2.11 (m, 3H), 2.07-1.97 (m, 4H), 1.97-1.81 (m, 7H), 1.76 (s, 3H), 1.71-1.48 (m, 6H), 1.35-1.18 (m, 3H), 1.15-1.03 (m, 2H), 0.93-0.86 (m, 2H), 0.85-0.78 (m, 2H). ¹⁹F NMR (377 MHZ, METHANOL-d4) δ=−77.009.

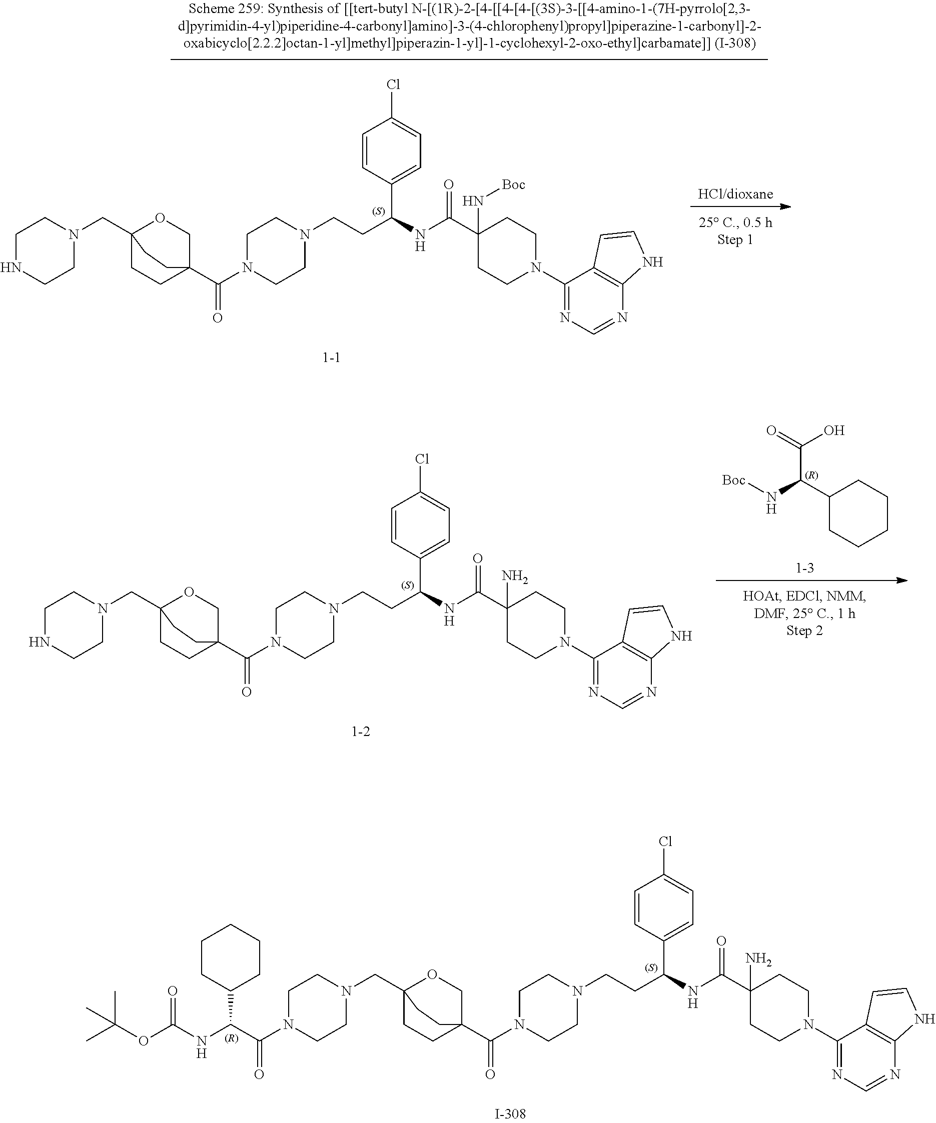

### Step 1: Synthesis of 1-2

A solution of 1-1 (20 mg, 24.00 μmol, 1 eq) in DCM (0.3 mL) was added HCl/dioxane (2 M, 0.2 mL, 16.67 eq). The mixture was stirred at 25° C. for 0.5 hr. The reaction mixture was concentrated under reduced pressure to give product. 1-2 (20 mg, crude, HCl) as a white solid. LCMS (Method D): R^{t}=0.197 min, [M+H]⁺=733.5.

### Step 2: Synthesis of I-308

To a solution of 1-2 (20 mg, 25.98 μmol, 1 eq, HCl) and 1-3 (6.69 mg, 25.98 μmol, 1 eq) in DMF (0.2 mL) was added EDCI (14.94 mg, 77.94 μmol, 3 eq), HOAt (3.54 mg, 25.98 μmol, 3.63 μL, 1 eq) and NMM (13.14 mg, 129.90 μmol, 14.28 μL, 5 eq). The mixture was stirred at 25° C. for 1 hr. showed 1-2 was consumed completely and desired mass. The reaction mixture was concentrated under reduced pressure to give a residue. The residue was purified by reversed phase column (Neutral condition) and lyophilized to give I-308 (6.44 mg, 6.62 μmol, 25.48% yield, 100% purity), which as a white solid. LCMS: R^{t}=0.290 min, [M+H]⁺=972.6. SFC: R^{t}=1.232 min. ¹H NMR (400 MHZ, METHANOL-d4) δ=8.12 (s, 1H), 7.32 (d, J=1.2 Hz, 4H), 7.12 (d, J=3.6 Hz, 1H), 6.62 (d, J=3.6 Hz, 1H), 4.98 (t, J=6.4 Hz, 1H), 4.56-4.44 (m, 2H), 4.34 (d, J=7.6 Hz, 1H), 3.94 (s, 2H), 3.70 (s, 4H), 3.67-3.59 (m, 4H), 3.56-3.48 (m, 1H), 2.57 (s, 2H), 2.53-2.43 (m, 4H), 2.43-2.31 (m, 4H), 2.29 (s, 2H), 2.26-2.13 (m, 4H), 2.13-1.99 (m, 2H), 1.98-1.93 (m, 2H), 1.92-1.83 (m, 2H), 1.75 (s, 3H), 1.69-1.54 (m, 7H), 1.43 (s, 9H), 1.36-1.10 (m, 4H), 1.10-0.96 (m, 2H).

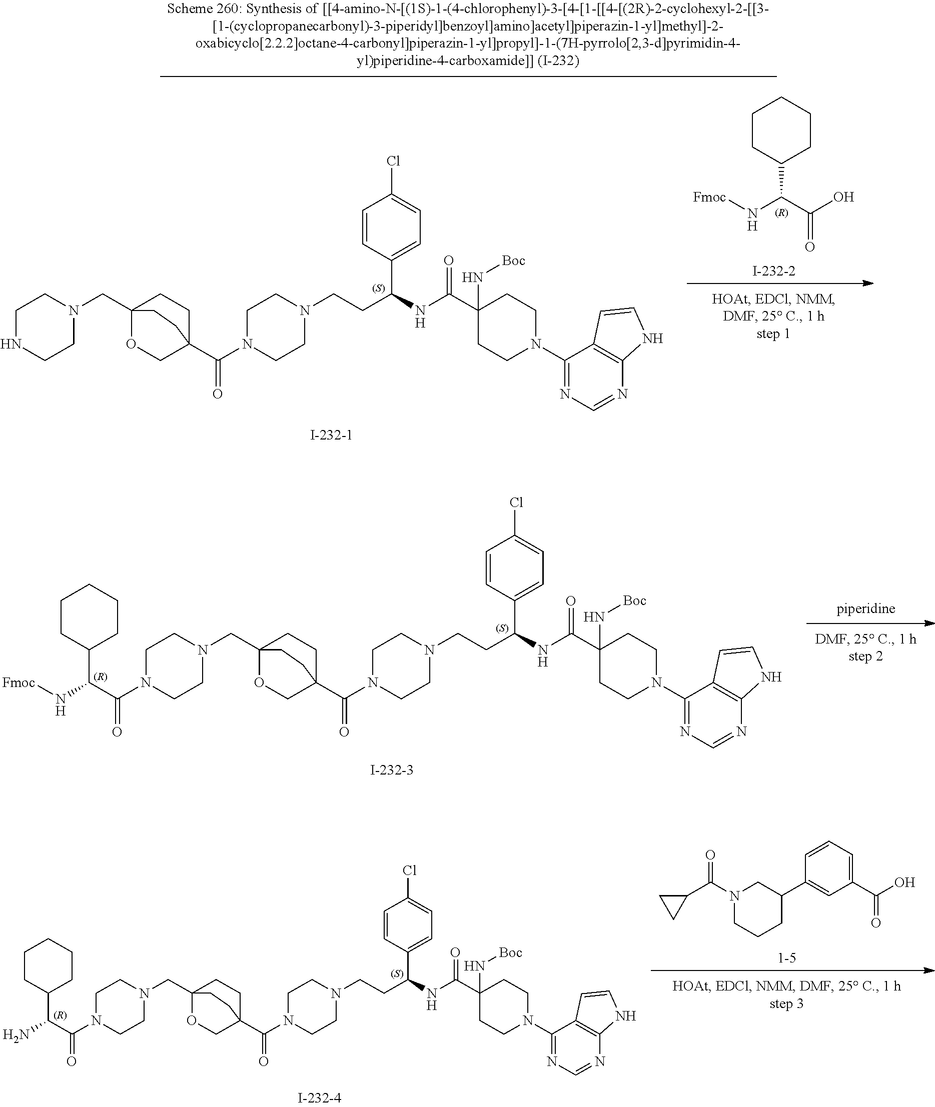

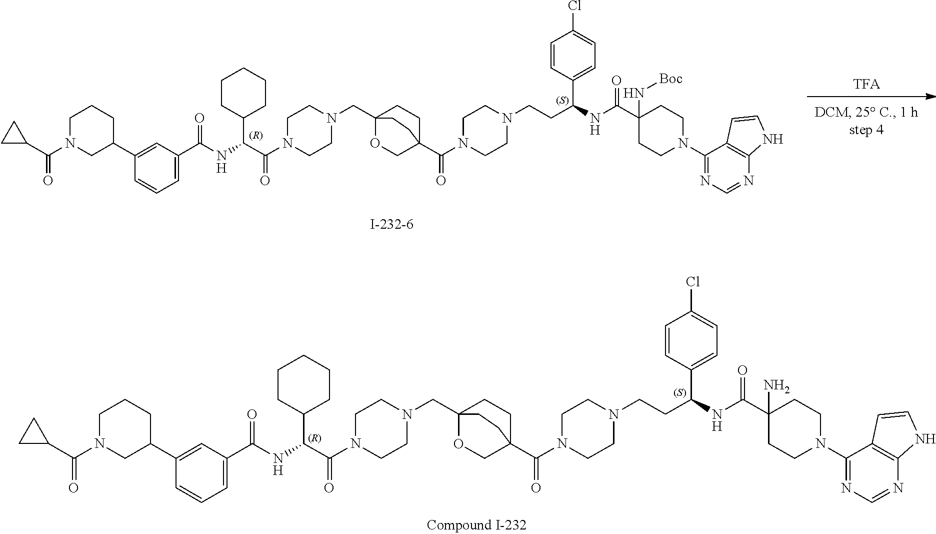

### Step 1: Synthesis of I-232-3

To a solution of I-232-1 (70 mg, 83.99 μmol, 1 eq) and I-232-2 (31.87 mg, 83.99 μmol, 31.87 μL, 1 eq) in DMF (1 mL) was added EDCI (48.30 mg, 251.96 μmol, 3 eq), HOAT (11.43 mg, 83.99 μmol, 11.75 μL, 1 eq) and NMM (42.48 mg, 419.94 μmol, 46.17 μL, 5 eq). The mixture was stirred at 25° C. for 1 hr. showed I-232-1 was consumed completely and 66% of desired mass. The reaction mixture was concentrated under reduced pressure to give a residue. The residue was purified by flash silica gel chromatography (ISCO®; 12 g SepaFlash® Silica Flash Column, Eluent of 0˜60% Ethyl acetate/Methanol gradient (@ 40 mL/min) and concentrated under reduced pressure to give product. I-232-3 (110 mg, 67.20 μmol, 80.02% yield, 73% purity) as a light yellow solid. LCMS (Method D): R^{t}=0.364 min, [M+H]⁺=1194.8.

### Step 2: Synthesis of I-232-4

To a solution of I-232-3 (90 mg, 75.32 μmol, 1 eq) in DMF (1 mL) was added piperidine (12.83 mg, 150.64 μmol, 14.88 μL, 2 eq). The mixture was stirred at 25° C. for 1 hr. showed I-232-3 was consumed completely and 41% of desired mass. The reaction mixture was concentrated under reduced pressure to give a residue. The residue was purified by reversed phase column (Neutral condition) and lyophilized to give product. I-232-4 (65 mg, 55.47 μmol, 73.64% yield, 83% purity) as a white solid. LCMS: R^{t}=0.665 min, [M+H]⁺=972.2.

### Step 3: Synthesis of I-232-6

To a solution of I-232-4 (55 mg, 56.55 μmol, 1 eq) and I-232-5 (15.46 mg, 56.55 μmol, 1 eq) in DMF (0.5 mL) was added EDCI (32.52 mg, 169.64 μmol, 3 eq), HOAT (7.70 mg, 56.55 μmol, 7.91 μL, 1 eq) and NMM (28.60 mg, 282.73 μmol, 31.08 μL, 5 eq). The mixture was stirred at 25° C. for 1 hr. showed I-232-4 was consumed completely and 79% of desired mass. The reaction mixture was concentrated under reduced pressure to give a residue. The residue was purified by flash silica gel chromatography (ISCO®; 12 g SepaFlash® Silica Flash Column, Eluent of 0˜65% Ethylacetate/Methanol gradient @ 40 mL/min) and concentrated under reduced pressure to give product. I-232-6 (67 mg, 50.20 μmol, 88.77% yield, 92% purity) as a light yellow solid. LCMS (Method D): R^{t}=0.355 min, [M+H]⁺=1227.9. SFC: R^{t}=1.820 min.

### Step 4: Synthesis of I-232

To a solution of I-232-6 (47 mg, 38.27 μmol, 1 eq) in DCM (0.5 mL) was added TFA (460.50 mg, 4.04 mmol, 0.3 mL, 105.52 eq). The mixture was stirred at 25° C. for 1 hr. showed I-232-6 was consumed completely and 72% of desired mass. The reaction mixture was concentrated in vacuum to give a residue. The residue was purified by reversed phase column (0.1% TFA) and lyophilized to give product. I-232 (20.5 mg, 16.51 μmol, 43.13% yield, 100% purity; TFA) as a white solid. LCMS (Method D): R^{t}=0.320 min, [M+H]⁺=1127.7. SFC: R^{t}=2.066 min. ¹H NMR (400 MHZ, METHANOL-d4) δ=8.37 (s, 1H), 7.82-7.75 (m, 1H), 7.75-7.68 (m, 1H), 7.55-7.40 (m, 2H), 7.40-7.31 (m, 5H), 6.92 (d, J=3.6 Hz, 1H), 5.04-4.99 (m, 1H), 4.79 (d, J=8.4 Hz, 1H), 4.67-4.50 (m, 4H), 4.45-4.33 (m, 2H), 4.04 (s, 2H), 3.94-3.82 (m, 3H), 3.72-3.37 (m, 6H), 3.29-3.20 (m, 5H), 3.19-3.09 (m, 2H), 2.91-2.54 (m, 5H), 2.44-2.13 (m, 6H), 2.11-1.90 (m, 11H), 1.90-1.75 (m, 6H), 1.73-1.58 (m, 3H), 1.41-1.18 (m, 4H), 1.16-1.06 (m, 2H), 0.90-0.73 (m, 4H). ¹⁹F NMR (377 MHz, METHANOL-d4) δ=−76.971.

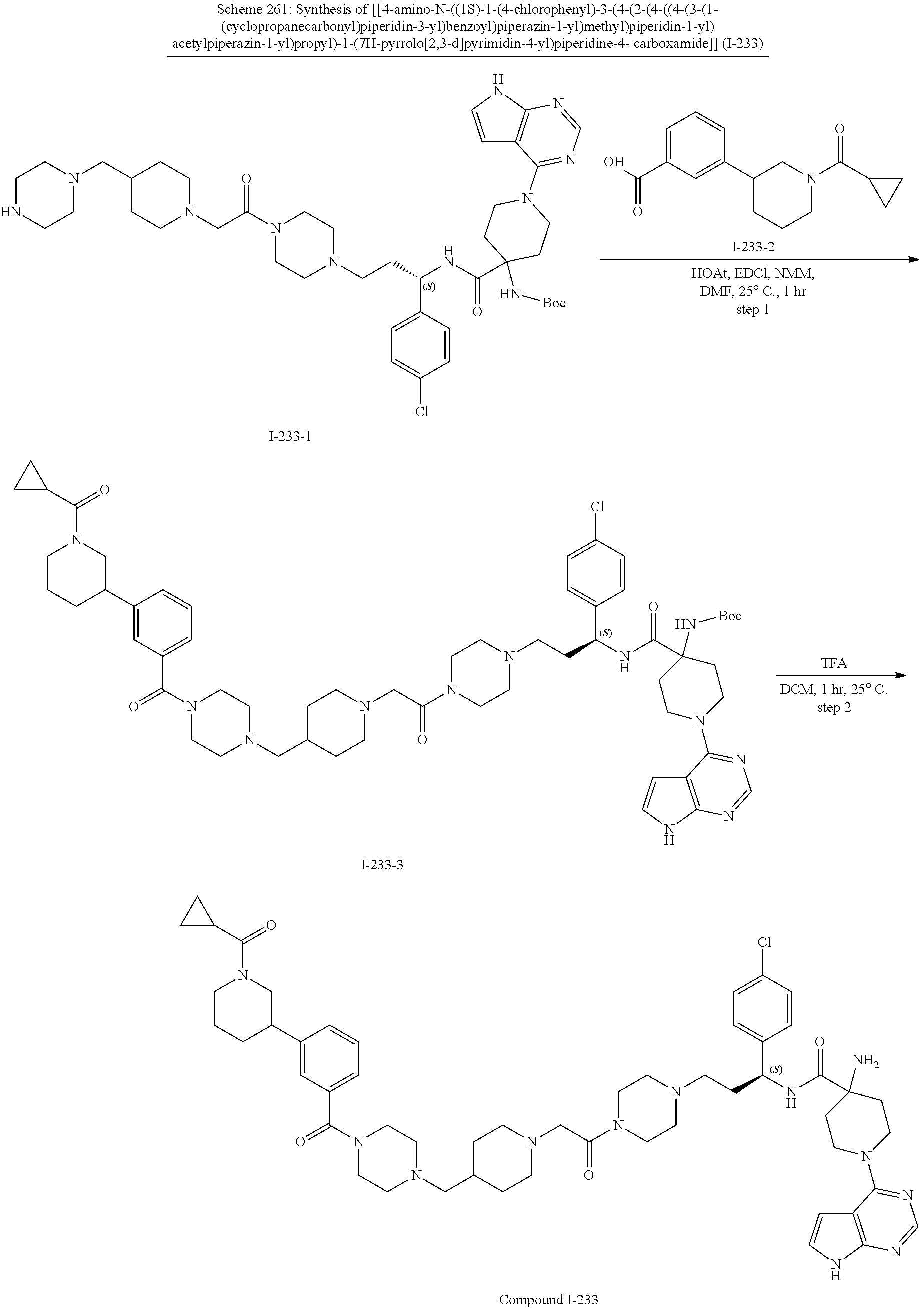

### Step 1: Synthesis of I-233-3

To a solution of I-233-2 (22.21 mg, 81.25 μmol, 1 eq) in DMF (1 mL) was added HOAt (11.06 mg, 81.25 μmol, 11.37 μL, 1 eq), EDCI (46.73 mg, 243.76 μmol, 3 eq), NMM (41.09 mg, 406.27 μmol, 44.67 μL, 5 eq) and I-233-1 (100 mg, 121.88 μmol, 1.5 eq). The mixture was stirred at 25° C. for 1 hr. The mixture was washed with water (1 mL) and extract with EA (2 mL*3). The combined organic layers dried over sodium sulfate, filtered, and concentrated under reduced pressure to give a residue. The residue was purified by reversed phase column (FA condition), the eluent was concentrated under reduced pressure to remove ACN and lyophilized to give product. I-233-3 (80 mg, 73.19 μmol, 90.07% yield, 98.414% purity) was obtained as white solid. LCMS (Method D): R^{t}=0.294 min, [M+H]⁺=1075.2. SFC: R^{t}: 3.785 min, 4.799 min.

### Step 2: Synthesis of I-233

To a solution of I-233-3 (70 mg, 65.07 μmol, 1 eq) in DCM (0.7 mL) was added TFA (0.14 mL). The mixture was stirred at 25° C. for 1 hr. The mixture was adjusted to a pH to 7˜8 with NaHCO₃, and the mixture was washed with water (1 mL) and extracted with DCM (1 mL*3). The combined organic layers dried over sodium sulfate, filtered, and concentrated under reduced pressure to give a residue. The residue was purified by prep-HPLC (column: CD02-Waters Xbidge BEH C18 150*25*10 um; mobile phase: [water (TFA)-ACN]; gradient: 5%-35% B over 10 min), the eluent was concentrated under reduced pressure to remove ACN and lyophilized to give product. The residue was further purified by prep-HPLC (column: CD07-Daisogel SP-100-8-ODS-PK 150*25*10 um; mobile phase: [water (NH4HCO3)-ACN];
gradient: 30%-60% B over 11 min), the eluent was concentrated under reduced pressure to remove ACN and lyophilized to give product. I-233 (23.74 mg, 24.33 μmol, 37.39% yield, 100% purity) was obtained as a white solid. LCMS (Method D): R^{t}=0.242 min, [M+H]⁺=975.7. ¹H NMR (400 MHZ, METHANOL-d4) δ=8.12 (s, 1H), 7.45-7.39 (m, 2H), 7.36 (s, 1H), 7.30-7.30 (m, 1H), 7.32 (s, 3H), 7.29-7.25 (m, 1H), 7.12 (d, J=3.6 Hz, 1H), 6.62 (d, J=3.6 Hz, 1H), 4.97 (m, 1H), 4.55-4.45 (m, 3H), 4.37 (d, J=9.2 Hz, 1H), 3.81-3.71 (m, 2H), 3.68-3.60 (m, 6H), 3.48-3.40 (m, 2H), 3.17 (s, 2H), 2.88 (d, J=11.2 Hz, 2H), 2.83-2.68 (m, 2H), 2.49 (d, J=3.2 Hz, 3H), 2.44 (d, J=4.4 Hz, 2H), 2.40-2.32 (m, 5H), 2.22 (d, J=7.2 Hz, 2H), 2.20-2.10 (m, 2H), 2.10-2.00 (m, 5H), 1.99-1.92 (m, 2H), 1.91-1.80 (m, 2H), 1.77 (s, 1H), 1.74 (s, 1H), 1.65-1.60 (m, 1H), 1.60-1.50 (m, 3H), 1.28-1.18 (m, 2H), 0.88 (d, J=4.4 Hz, 2H), 0.85-0.77 (m, 2H). SFC: R^{t}=3.124 min, 3.407 min.

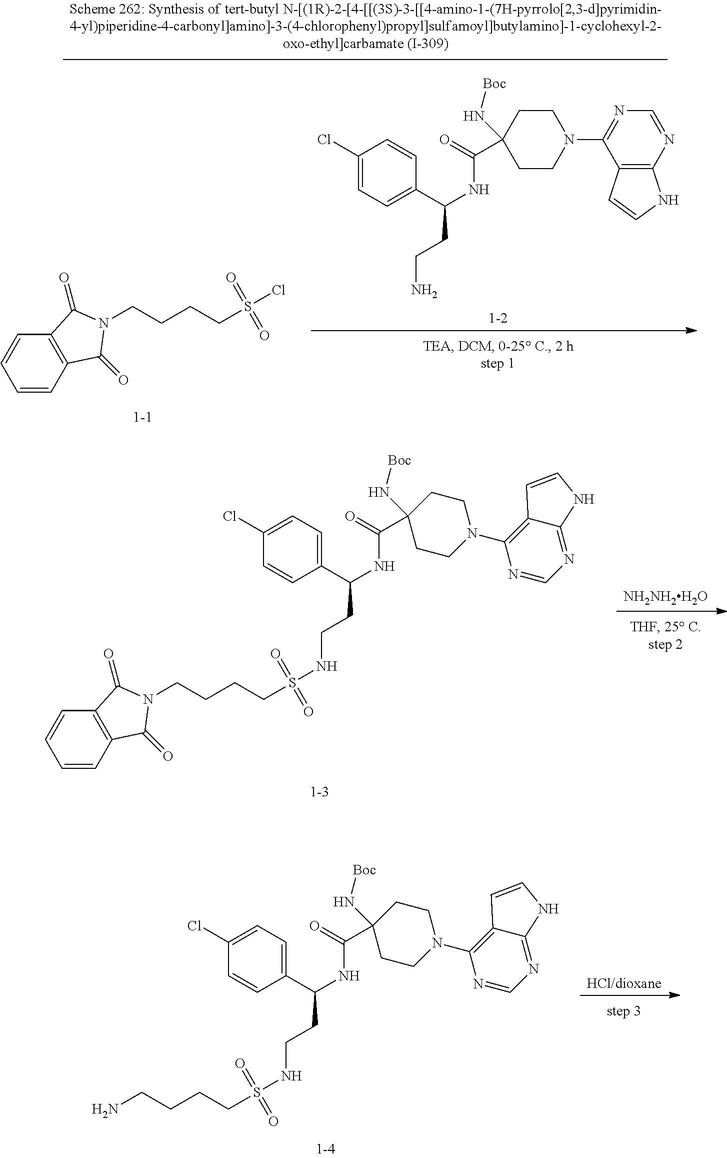

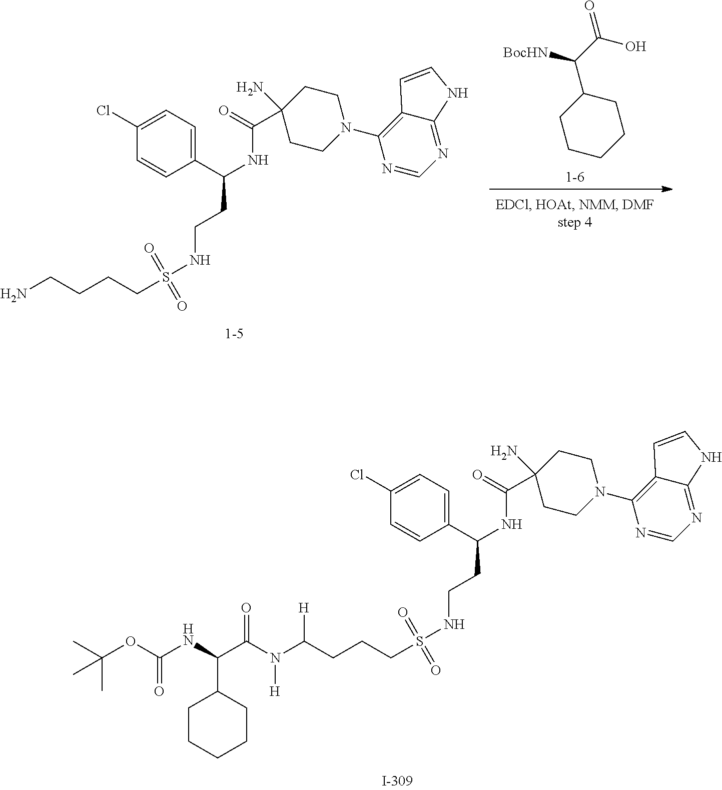

To a solution of 1-2 (0.9 g, 1.70 mmol, 1 eq) and TEA (517.40 mg, 5.11 mmol, 711.70 μL, 3 eq) in DCM (10 mL) was added 1-1 (617.15 mg, 2.05 mmol, 1.2 eq) at 0° C. Then the mixture was stirred at 25° C. for 2 hrs. The reaction mixture was adjusted to pH=5 by 1N HCl, extracted with EA (5 mL*3). The combined organic layers were washed with brine (5 mL*2), dried over anhydrous Na₂SO₄, filtered, and concentrated under reduced pressure to give a residue. The residue was purified by flash silica gel chromatography (ISCO®; 12 g SepaFlash® Silica Flash Column, Ethylacetate/Petroleum ether gradient @ 100 mL/min) and concentrated under vacuum. 1-3 (0.9 g, 1.03 mmol, 60.59% yield, 91.034% purity) was obtained as a green solid. LCMS: R^{t}=0.657 min, [M+H]⁺=793.4. SFC: R^{t}=1.915 min.

### Step 2: Synthesis of 1-4

To a solution of 1-3 (0.1 g, 126.05 μmol, 1 eq) in THF (1 mL) was added hydrazine: hydrate (44.17 mg, 882.36 μmol, 42.80 μL, 7 eq). The mixture was stirred at 25° C. for 16 hrs. as a major peak. The reaction mixture was quenched with H₂O (2 ml), extracted with EA (1 mL*3). The combined organic layers were washed with brine (5 mL*2), dried over anhydrous Na₂SO₄, filtered, and concentrated under reduced pressure to give the crude product. The aqueous phase was quenched by HCl solution (1 M). The crude product was triturated with 5 mL of PE:EA=50:1 at 25° C. for 30 mins, filtered, and the filter cake was concentrated under vacuum. 1-4 (50 mg, 75.39 μmol, 59.81% yield) was obtained as a white solid. LCMS: R^{t}=0.409 min, [M+H]⁺=663.2.

### Step 3: Synthesis of 1-5

To a solution of 1-4 (50 mg, 75.39 μmol, 1 eq) in DCM (0.2 mL) was added HCl/dioxane (4 M, 0.2 mL, 10.61 eq). The mixture was stirred at 25° C. for 1 h. The mixture was concentrated under vacuum to give a crude product. The crude was used for next step directly without purification. 1-5 (40 mg, crude, HCl) was obtained as a white solid. LCMS: R^{t}=0.342 min, [M+H]⁺=563.3.

### Step 4: Synthesis of I-309

To a solution of 1-5 (40 mg, 66.71 μmol, 1 eq, HCl) in DMF (0.5 mL) was added 1-6 (17.17 mg, 66.71 μmol, 1 eq), EDCI (25.58 mg, 133.43 μmol, 2 eq), NMM (33.74 mg, 333.57 μmol, 36.67 μL, 5 eq) and HOAt (9.08 mg, 66.71 μmol, 9.33 μL, 1 eq). The mixture was stirred at 25° C. for 1 hr. The mixture was diluted with water (2 mL), extracted with EtOAc (5 mL+3). The organic layer was concentrated under vacuum. The residue was purified by Prep-HPLC (column: Waters Xbridge 150*25 mm*5 um; mobile phase: [water (NH₄HCO3)-ACN]; gradient: 30%-60% B over 15 min) and dried by lyophilization to give I-309 (20 mg, 24.92 μmol, 37.36% yield) as white solid. LCMS: R^{t}=0.463 min, [M+H]⁺=802.4. SFC: R^{t}=1.893 min. ¹H NMR (400 MHZ, DMSO-d₆) δ=11.64 (br s, 1H), 8.38 (br d, J=8.4 Hz, 1H), 8.11 (s, 1H), 7.86 (br t, J=5.2 Hz, 1H), 7.38-7.33 (m, 4H), 7.17-7.12 (m, 1H), 7.06-7.01 (m, 1H), 6.60-6.50 (m, 2H), 4.84 (br d, J=6.4 Hz, 1H), 4.41-4.32 (m, 2H), 3.76-3.71 (m, 1H), 3.59-3.50 (m, 2H), 3.12-3.05 (m, 1H), 3.05-3.00 (m, 1H), 3.00-2.95 (m, 2H), 2.94-2.89 (m, 2H), 2.21-2.05 (m, 2H), 2.02-1.96 (m, 1H), 1.96-1.92 (m, 1H), 1.92-1.87 (m, 1H), 1.87-1.82 (m, 1H), 1.65-1.59 (m, 4H), 1.58-1.51 (m, 3H), 1.50-1.46 (m, 3H), 1.42 (br d, J=6.8 Hz, 2H), 1.36 (s, 9H), 1.08 (br s, 3H), 0.99-0.88 (m, 2H).

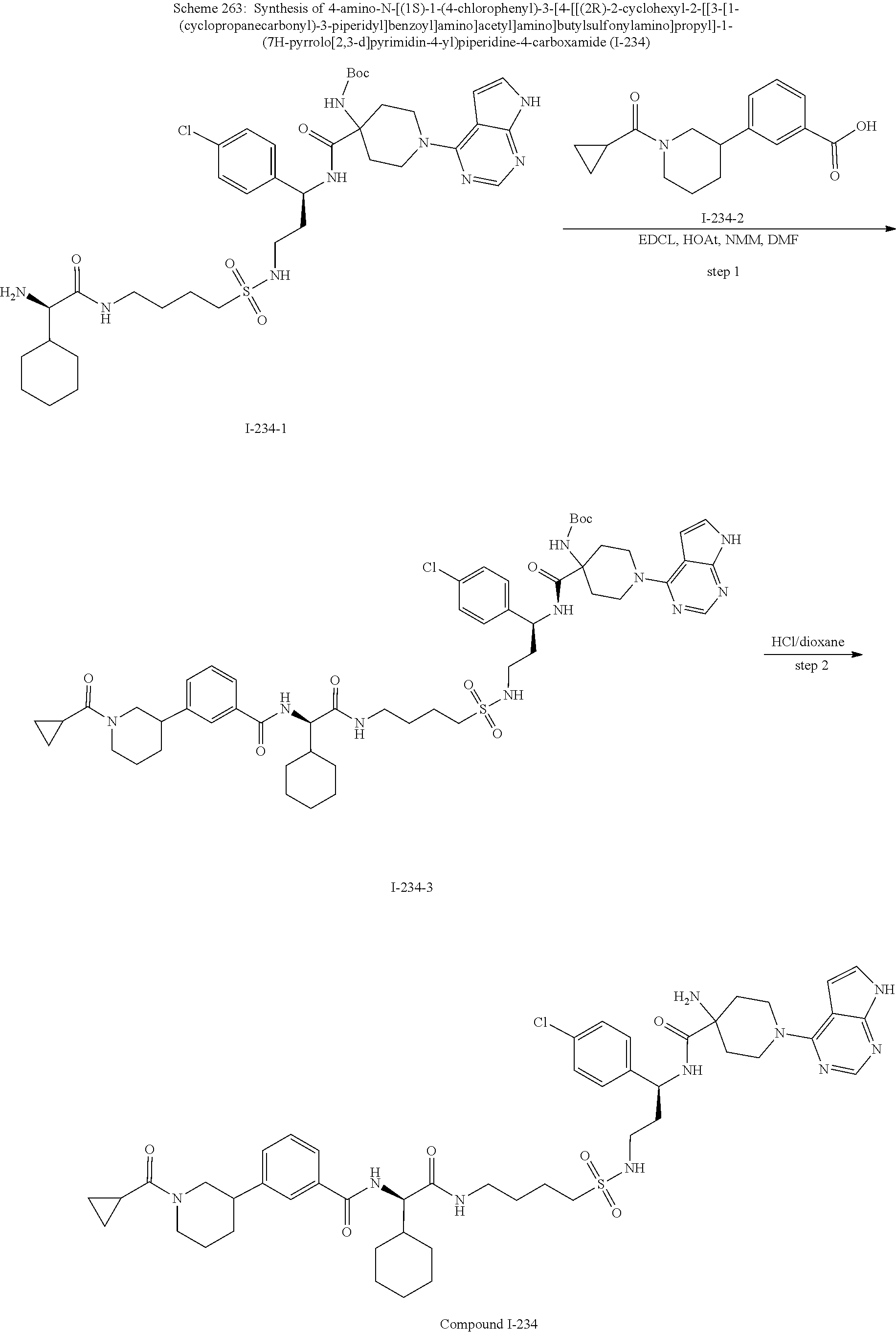

### Step 1: Synthesis of I-234-3

To a solution of I-234-1 (40 mg, 49.85 μmol, 1 eq) in DMF (0.4 mL) was added HOAt (6.78 mg, 49.85 μmol, 6.97 μL, 1 eq) and EDCI (19.11 mg, 99.70 μmol, 2 eq), NMM (25.21 mg, 249.24 μmol, 27.40 μL, 5 eq) and I-234-2 (13.63 mg, 49.85 μmol, 1 eq). The mixture was stirred at 25° C. for 1 hr. The reaction mixture was poured into water (1.5 ml), filtered, and the filter cake was washed with water (1 mL*3), the filter cake was dried under vacuum to give a residue. The crude product was purified by reversed phase column (0.1% NH₃·H₂O) and lyophilization to give product. I-234-3 (25 mg, 23.58 μmol, 47.30% yield, 99.76% purity) was obtained as a white solid. LCMS: R^{t}=0.702 min, [M+H]⁺=1057.8. SFC: R^{t}=1.139 min, 1.374 min.

### Step 2: Synthesis of I-234

To a solution of I-234-3 (25 mg, 23.64 μmol, 1 eq) in DCM (0.2 mL) was added HCl/dioxane (4 M, 0.05 mL, 8.46 eq). The mixture was stirred at 25° C. for 1 hr. as a major peak. The mixture was concentrated under vacuum to give a residue. The residue was diluted with MeOH (0.5 ml) and H₂O (20 ml) and freeze-dried to give product. I-234 (11.42 mg, 11.06 μmol, 46.79% yield, 96.271% purity, HCl) was obtained as a white solid. LCMS: R^{t}=0.640 min, [M+H]⁺=957.7. SFC: R^{t}=1.899 min, 2.278 min. ¹H NMR (400 MHZ, METHANOL-d4) δ=8.24 (s, 1H), 7.74-7.56 (m, 2H), 7.39-7.20 (m, 7H), 6.79 (br s, 1H), 4.97-4.99 (m, J, 1H), 4.62-4.51 (m, 2H), 4.43 (br d, J=8.8 Hz, 1H), 4.31-4.21 (m, 1H), 4.17 (br d, J=8.4 Hz, 1H), 3.78-3.64 (m, 2H), 3.03-2.91 (m, 4H), 2.74-2.46 (m, 4H), 2.10-1.90 (m, 5H), 1.86-1.53 (m, 15H), 1.25-0.96 (m, 7H), 0.81-0.66 (m, 4H).

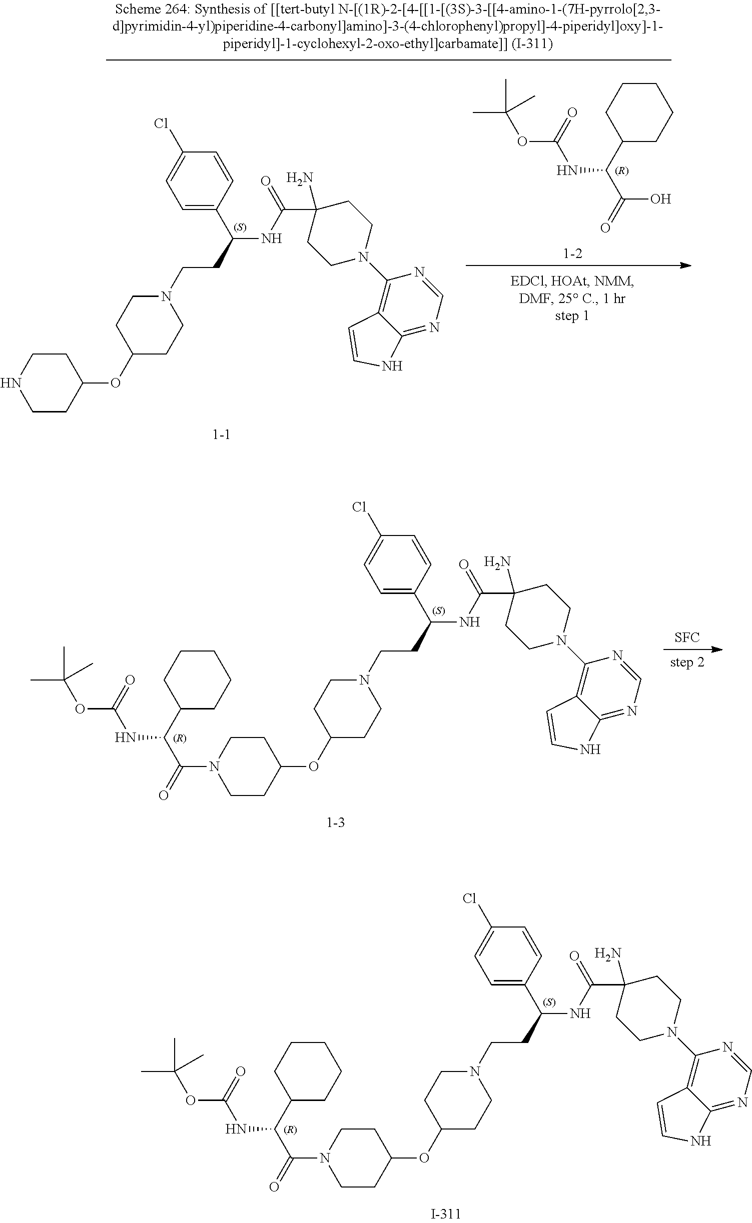

### Step 1: Synthesis of 1-3

To a solution of 1-1 (150 mg, 252.03 μmol, 1 eq) in DMF (1.5 mL) was added EDCI (144.94 mg, 756.08 μmol, 3 eq), NMM (127.46 mg, 1.26 mmol, 138.54μ, 5 eq) and 1-2 (64.85 mg, 252.03 μmol, 1 eq) and HOAt (34.30 mg, 252.03 μmol, 35.26 μL, 1 eq). The mixture was stirred at 25° C. for 0.5 hr. The crude product was purified by reversed phase column (0.1% FA condition) and the eluent was lyophilized to give the product. 1-3 (75.61 mg, 85.87 μmol, 34.07% yield, 100% purity, FA) was obtained as a white solid. LCMS (Method D): R^{t}=0.348 min, [M+H]⁺=834.4. SFC: R^{t}=1.263 min. ¹H NMR (400 MHZ, DMSO-d₆) δ=11.70 (s, 1H), 8.83-8.51 (m, 1H), 8.14 (d, J=1.6 Hz, 1H), 7.42-7.29 (m, 4H), 7.22-7.13 (m, 1H), 6.84-6.69 (m, 1H), 6.62 (d, J=1.6 Hz, 1H), 4.84 (t, J=6.4 Hz, 1H), 4.48 (d, J=9.2 Hz, 2H), 4.29-4.08 (m, 1H), 3.99-3.73 (m, 2H), 3.67-3.59 (m, 1H), 3.57-3.40 (m, 5H), 3.14-2.92 (m, 2H), 2.86-2.71 (m, 2H), 2.39 (s, 2H), 2.35-2.23 (m, 2H), 2.15-2.00 (m, 2H), 1.97-1.86 (m, 2H), 1.78 (s, 3H), 1.70-1.55 (m, 7H), 1.50 (d, J=10.4 Hz, 3H), 1.36 (s, 10H), 1.11 (s, 3H), 1.01-0.87 (m, 2H).

### Step 2: Synthesis of I-311

The 1-3 (75.61 mg, 85.87 μmol, 34.07% yield, 100% purity, FA) was purified by SFC silica gel chromatography (column: DAICEL CHIRALCEL OJ (250 mm*30 mm, 10 um); mobile phase: [CO₂-MeOH (0.1% NH3H₂O)]; B %: 15%, isocratic elution mode) and concentrated under reduced pressure to give crude product. The crude product was purified by reversed phase column (0.1% FA condition) and the eluent was lyophilized to give the product. I-311 (25.3 mg, 28.73 μmol, 39.96% yield, 100% purity, FA) was obtained as a white solid. LCMS (Method D): R^{t}=0.323 min, [M+H]⁺=834.5. SFC: R^{t}=1.263 min. ¹H NMR (400 MHz, METHANOL-d4) δ=8.51 (s, 1H), 8.13 (s, 1H), 7.35 (s, 4H), 7.13 (d, J=4.0 Hz, 1H), 6.62 (d, J=3.6 Hz, 1H), 4.93 (t, J=7.2 Hz, 1H), 4.56-4.44 (m, 2H), 4.37 (d, J=8.0 Hz, 1H), 4.03-3.87 (m, 1H), 3.86-3.71 (m, 2H), 3.70-3.60 (m, 3H), 3.55-3.33 (m, 2H), 3.28-3.18 (m, 1H), 3.12-2.97 (m, 2H), 2.84-2.62 (m, 4H), 2.26-2.10 (m, 4H), 1.94 (d, J=1.6 Hz, 3H), 1.76 (d, J=8.8 Hz, 6H), 1.69-1.64 (m, 2H), 1.59 (d, J=13.2 Hz, 4H), 1.43 (s, 9H), 1.31-1.15 (m, 3H), 1.11-0.96 (m, 2H).

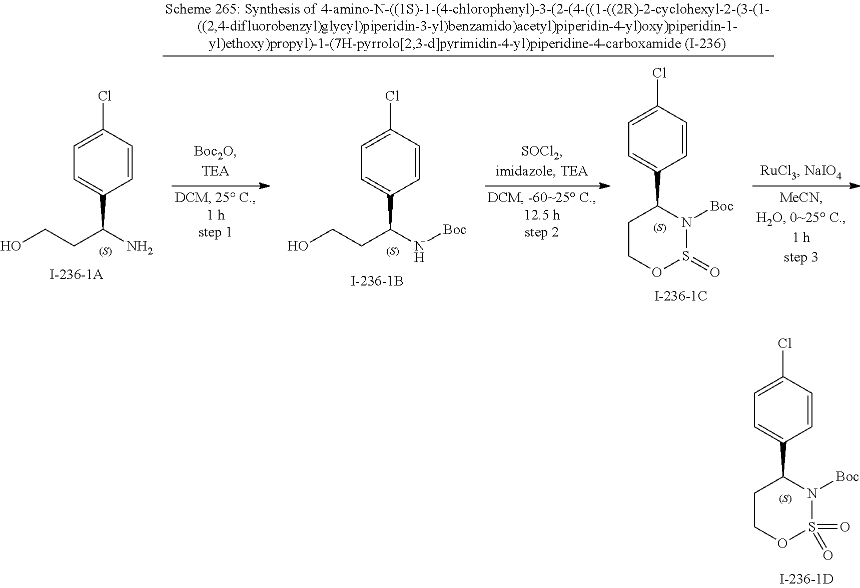

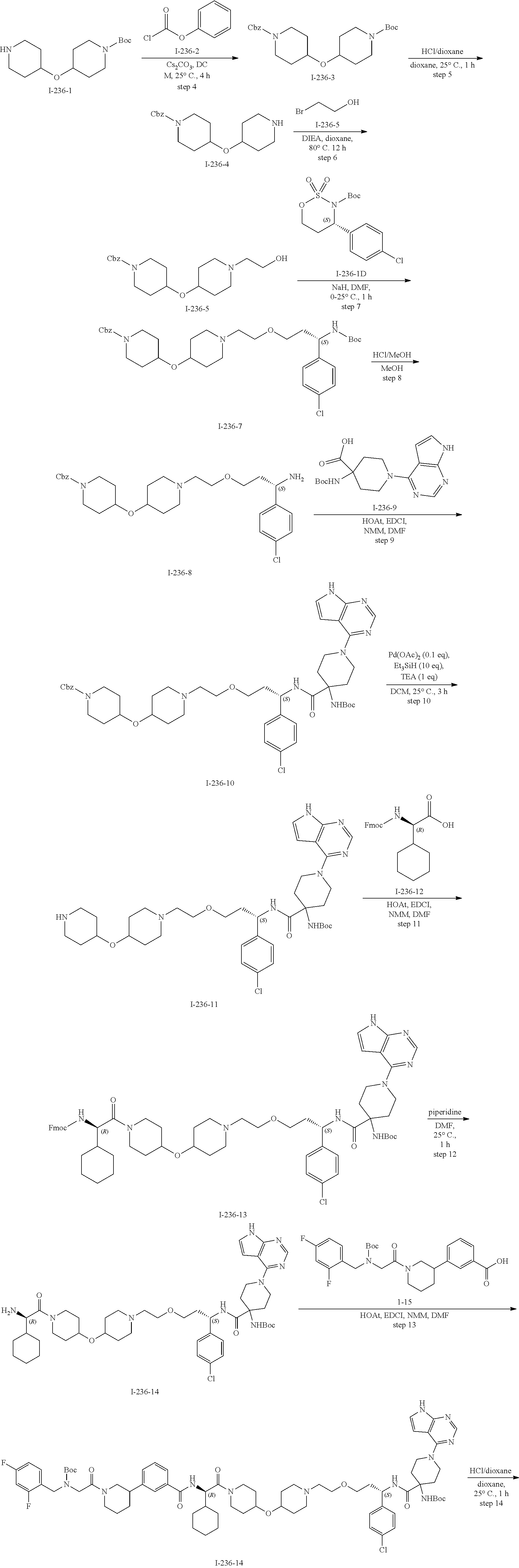

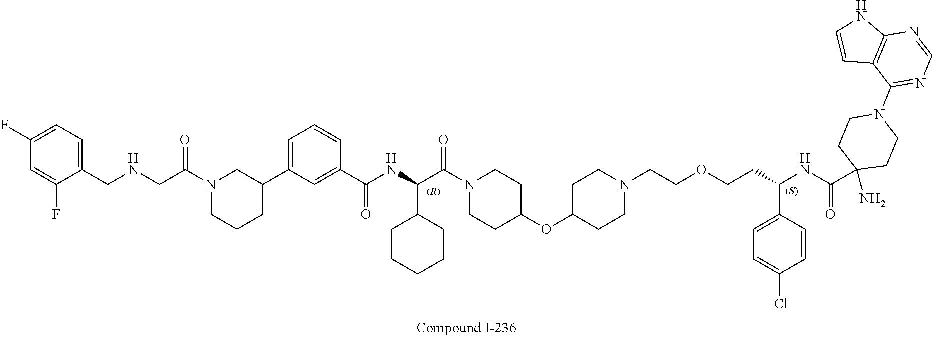

### Step 1: Synthesis of I-236-1B

To a solution of I-236-1A (15 g. 80.80 mmol, 1 eq) in DCM (150 mL) was added Boc₂O (21.16 g. 96.96 mmol, 22.27 mL, 1.2 eq) and TEA (24.53 g, 242.39 mmol, 33.74 mL, 3 eq). The mixture was stirred at 25° C. for 1 hr. The reaction was diluted with H₂O (50 mL), extracted with DCM 300 mL (100 mL*3), the organic phase was washed with saturated aqueous NaCl (50 mL). Then dried over [Na₂SO₄], filtered, and concentrated under reduced pressure to give a residue. The crude product was purified by re-crystallization from PE (100 mL) at 20° C. The filtered, and concentrated under reduced pressure to give a residue as white solid. I-236-1B (19.5 g. 64.35 mmol, 79.64% yield, 94.3% purity). LCMS: R^{t}=0.511 min, [M+Na]⁺=308.1.

### Step 2: Synthesis of I-236-1C

To a solution of imidazole (18.58 g, 272.95 mmol, 4 eq) and TEA (15.19 g, 150.12 mmol, 20.90 mL, 2.2 eq) in DCM (150 mL) was cooled to −60° C., then added SOCl₂ (9.74 g, 81.89 mmol, 5.95 mL, 1.2 eq). The mixture was stirred at −60° C. for 0.5 hr. Then a solution of I-236-1B (19.5 g, 68.24 mmol, 1 eq) in DCM (100 mL) was added. The reaction mixture was then stirred at 25° C. for 12 hr. The reaction mixture was poured into water (200 mL) and extracted with DCM (150 mL*2), the organic layer was washed with brine (100 mL) and dried over Na₂SO₄ and concentrated to give product. I-236-1C (22 g, 66.30 mmol, 97.16% yield) as off-white solid. 1HNMR (400 MHZ, DMSO-d₆) δ=7.44 (d, J=8.4 Hz, 2H), 7.31 (d, J=8.4 Hz, 2H), 5.32-5.25 (m, 1H), 4.44-4.29 (m, 1H), 3.82 (m, 1H), 3.03-2.83 (m, 1H), 2.32 (m, 1H), 1.37 (s, 9H).

### Step 3: Synthesis of I-236-1D

To a solution of I-236-1C (22 g, 66.30 mmol, 1 eq) in MeCN (200 mL) was added RuCl₃ (1.38 g, 6.63 mmol, 442.22 μL, 0.1 eq), H₂O (200 mL) and NaIO4 (15.60 g, 72.93 mmol, 4.04 mL, 1.1 eq) at 0° C. The mixture was stirred at 25° C. for 1 hr. The reaction was diluted with Sat. NaHCO₃ (100 mL), extracted with ethyl acetate 300 mL (100 mL+3), The organic phase was washed with saturated aqueous NaCl (50 mL). Then dried over (Na₂SO₄), filtered, and concentrated under reduced pressure to give a crude product. The aqueous phase was quenched by Sat. Na₂S203 (100 mL). The crude product was triturated with PE:EA=5:1 (100 mL) at 25° C. for 30 mins to give product. I-236-1D (18.255 g, 50.91 mmol, 76.79% yield, 97% purity) as off-white solid. LCMS: R^{t}=0.554 min, [2M+Na]⁺=717.1. ¹H NMR (400 MHZ, DMSO-d6) δ=7.54-7.45 (m, 2H), 7.43-7.36 (m, 2H), 5.65 (m, 1H), 4.78-4.64 (m, 1H), 4.41 (m, 1H), 2.80-2.67 (m, 1H), 2.65-2.55 (m, 1H), 1.41 (s, 9H).

### Step 4: Synthesis of I-236-3

To a solution of I-236-1 (20 g, 70.33 mmol, 1 eq) in DCM (200 mL) was added Cs₂CO₃ (45.83 g, 140.65 mmol, 2 eq) and I-236-2 (14.40 g, 84.39 mmol, 12.05 mL, 1.2 eq). The mixture was stirred at 25° C. for 4 hr. The reaction mixture was concentrated to get a residue. The residue was purified by flash silica gel chromatography (ISCO®; 220 g SepaFlash® Silica Flash Column, Eluent of 0˜60% Ethyl acetate/Petroleum ether gradient @ 100 mL/min) and the eluent was concentrated. I-236-3 (9 g, 21.50 mmol, 30.58% yield) as colorless oil. ¹H NMR (400 MHZ, CHLOROFORM-d) δ=7.41-7.35 (m, 5H), 5.13 (s, 2H), 3.89-3.69 (m, 4H), 3.65-3.48 (m, 2H), 3.31-3.17 (m, 2H), 3.10 (m, 2H), 1.89-1.70 (m, 4H), 1.51 (m, 4H), 1.46 (s, 9H).

### Step 5: Synthesis of I-236-4

To a solution of I-236-3 (9 g, 21.50 mmol, 1 eq) in dioxane (50 mL) was added HCl/dioxane (4 M, 50 mL, 9.30 eq). The mixture was stirred at 25° C. for 1 hr. The reaction mixture was concentrated to I-236-4 (6.6 g, 18.60 mmol, 86.49% yield, HCl) as white solid. ¹H NMR (400 MHZ, DMSO-d₆) δ=9.04 (br d, J=1.6 Hz, 2H), 7.44-7.25 (m, 5H), 5.06 (s, 2H), 3.80-3.56 (m, 4H), 3.11 (br s, 4H), 2.92 (br s, 2H), 2.00-1.83 (m, 2H), 1.82-1.72 (m, 2H), 1.71-1.58 (m, 2H), 1.42-1.27 (m, 2H).

### Step 6: Synthesis of I-236-6

To a solution of I-236-4 (2 g, 5.64 mmol, 1 eq, HCl) in dioxane (20 mL) was added DIEA (3.64 g, 28.18 mmol, 4.91 mL, 5 eq) and I-236-5 (1.06 g, 8.45 mmol, 599.22 μL, 1.5 eq). The mixture was stirred at 80° C. for 12 hr. The mixture was concentrated to get a residue. The residue was purified by reversed phase HPLC (ISCO®; 80 g SepaFlash® C18 Column, Eluent of 0˜90% (0.1% NH₃·H₂O) water/MeCN @ 90 mL/min) and lyophilization to give product. I-236-6 (1.6 g. 4.15 mmol, 73.62% yield, 94% purity) as yellow oil. LCMS: R^{t}=0.541 min, [M+H]⁺=363.3.

### Step 7: Synthesis of I-236-7

To a solution of I-236-6 (1 g. 2.76 mmol, 1 eq) in DMF (6 mL) was cooled to 0° C. under N₂ atmosphere, then added NaH (134.00 mg, 3.35 mmol, 60% purity; 1.21 eq) at 0° C. The mixture was stirred at 0° C. for 0.5 hr, the mixture of I-236-1D (959.59 mg, 2.76 mmol, 1 eq) in DMF (4 mL) was added into the reaction. The mixture was warm to 25° C., and stirred for 1 hr. The reaction mixture was quenched by water (1 mL) to get a mixture. The mixture was purified by reversed phase (26.8*125 mm, 120 g of XB—C18, 20-40 μm, 120 Å) Mobile phase: A for H₂O (0.1% HCl v/v) and B for acetonitrile; Gradient: B 5%-75% in 30 min: Flow rate: 60 mL/min; Column temperature: R.T. Wavelength: 220 nm/254 nm) then lyophilization to give product. I-236-7 (600 mg, 828.29 μmol, 30.02% yield, 87% purity) as white solid. LCMS: R^{t}=0.545 min, [M+H]⁺=630.3.

### Step 8: Synthesis of I-236-8

To a solution of intermediate 1-7 (600 mg, 952.06 μmol, 1 eq) in MeOH (6 mL) was added HCl/MeOH (4 M, 5.00 mL, 21.01 eq). The mixture was stirred at 25° C. for 1 hr. The reaction mixture was concentrated to give I-236-8 (540 mg, 924.53 μmol, 97.11% yield, 97% purity, HCl), which as gray oil. LCMS: R^{t}=0.425 min, [M+H]⁺=530.3. SFC: R^{t}=2.282 min.

### Step 9: Synthesis of I-236-10

To a solution of I-236-8 (540 mg, 953.12 μmol, 1 eq, HCl) and I-236-9 (344.45 mg, 953.12 μmol, 1 eq) in DMF (6 mL) was added HOAt (194.60 mg, 1.43 mmol, 200.00 μL, 1.5 eq), EDCI (365.43 mg, 1.91 mmol, 2 eq) and NMM (482.03 mg, 4.77 mmol, 523.94 μL, 5 eq). The mixture was stirred at 25° C. for 1 hr. The reaction mixture was quenched by water (1 mL) to get a mixture. The mixture was purified by reversed phase HPLC (ISCO®; 80 g SepaFlash® C18 Column, Eluent of 0˜90% (0.1% HCl) water/MeCN @ 100 mL/min) and lyophilization to give product. I-236-10 (590 mg, 668.71 μmol, 70.16% yield, 99% purity) as white solid. LCMS: R^{t}=0.477 min, [M+H]⁺=873.5. ¹H NMR (400 MHZ, CHLOROFORM-d) δ=10.04-9.83 (m, 1H), 8.50 (s, 1H), 8.27 (s, 1H), 7.86 (br d, J=6.8 Hz, 1H), 7.39-7.29 (m, 5H), 7.25-7.19 (m, 2H), 7.04 (d, J=3.2 Hz, 1H), 6.52 (d, J=3.6 Hz, 1H), 5.13-5.07 (m, 1H), 4.52-4.30 (m, 2H), 3.92-3.76 (m, 2H), 3.75-3.54 (m, 6H), 3.48-3.38 (m, 2H), 3.32-3.06 (m, 6H), 3.04-2.90 (m, 5H), 2.88-2.71 (m, 2H), 2.36-2.26 (m, 1H), 2.23 (br s, 2H), 2.20-2.10 (m, 2H), 2.10-1.93 (m, 3H), 1.87-1.69 (m, 4H), 1.60-1.49 (m, 2H), 1.47 (s, 9H).

### Step 10: Synthesis of I-236-11

To a solution of I-236-10 (170 mg, 194.62 μmol, 1 eq) in DCM (1.7 mL) was added Pd(OAc)₂ (4.37 mg, 19.46 μmol, 0.1 eq), TEA (29.54 mg, 291.94 μmol, 40.63 μL, 1.5 eq) and Et₃SiH (226.30 mg, 1.95 mmol, 310.86 μL, 10 eq) at 0° C. The mixture was stirred at 25° C. for 3 hr. The reaction mixture was concentrated to get a residue. The residue was purified by reversed phase HPLC (ISCO®; 40 g SepaFlash® C18 Column, Eluent of 0˜95% (0.1% NH3·H₂O) water/MeCN @ 60 mL/min) and lyophilization to give a white solid. Then the white solid was dissolved into 10% of NaHCO₃ aqueous solution (5 mL), extracted with (CHCl₃: i-PrOH=3:1) (5 mL*10), collected the organic phase, dried over Na₂SO₄ and concentrated to get product. I-236-11 (130 mg, 174.07 μmol, 89.44% yield, 99% purity) as a white solid. LCMS: R^{t}=0.412 min, [M+H]⁺=739.4. LCMS: R^{t}=0.399 min, [M+H]⁺=739.4.

### Step 11: Synthesis of I-236-13

To a solution of I-236-11 (130 mg, 175.83 μmol, 1 eq) and I-236-12 (66.72 mg, 175.83 μmol, 66.72 μL, 1 eq) in DMF (1.3 mL) was added HOAt (35.90 mg, 263.75 μmol, 36.90 μL, 1.5 eq), EDCI (67.41 mg, 351.66 μmol, 2 eq) and NMM (106.71 mg, 1.05 mmol, 115.99 μL, 6 eq). The mixture was stirred at 25° C. for 1 hr. The reaction was quenched by water (0.5 mL) to get a mixture. The mixture was purified by reversed phase HPLC (ISCO®; 12 g SepaFlash® C18 Column, Eluent of 0˜95% (0.1% FA) water/MeCN (@) 12 mL/min) and concentrated to give I-236-13 (150 mg, 132.18 μmol, 75.17% yield, 97% purity), which as white solid. LCMS: R^{t}=0.549 min, [M+H]⁺=1100.7. ¹H NMR (400 MHZ, CHLOROFORM-d) 8=10.24-10.01 (m, 1H), 8.26 (d, J=2.4 Hz, 1H), 7.90-7.80 (m, 1H), 7.77 (d, J=7.6 Hz, 2H), 7.64-7.56 (m, 2H), 7.44-7.36 (m, 2H), 7.35-7.28 (m, 3H), 7.24-7.19 (m, 2H), 7.05 (m, 1H), 6.52 (br s, 1H), 5.76-5.58 (m, 1H), 5.22-4.98 (m, 1H), 4.55 (m, 1H), 4.50-4.26 (m, 4H), 4.25-4.17 (m, 1H), 3.78-3.57 (m, 7H), 3.53-3.30 (m, 5H), 3.27-3.05 (m, 5H), 3.04-2.89 (m, 6H), 2.37-2.14 (m, 4H), 2.14-1.90 (m, 4H), 1.90-1.67 (m, 8H), 1.47 (s, 9H), 1.30-0.99 (m, 6H).

### Step 12: Synthesis of I-236-14

To a solution of I-236-13 (140 mg, 127.18 μmol, 1 eq) in DMF (1.4 mL) was added piperidine (32.49 mg, 381.55 μmol, 37.68 μL, 3 eq). The mixture was stirred at 25° C. for 1 hr. The reaction mixture was quenched by water (0.5 mL) to get a mixture. The mixture was purified by reversed phase HPLC (ISCO®; 30 g SepaFlash® C18 Column, Eluent of 0˜95% (0.1% FA) water/MeCN @ 16 mL/min) and lyophilization to give a solid. Then the solid was dissolved into 10% of NaHCO₃ aqueous solution (5 mL), extracted with (CHCl₃:i-PrOH=3:1) (5 mL*10), collected the organic phase, dried over Na₂SO₄ and concentrated to give I-236-14 (100 mg, 112.73 μmol, 88.64% yield, 99.04% purity), which as white solid. LCMS: R^{t}=0.438 min, [M+H]⁺=878.4.

### Step 13: Synthesis of I-236-16

To a solution of I-236-14 (100 mg, 113.83 μmol, 1 eq) and I-236-15 (55.61 mg, 113.83 μmol, 1 eq) in DMF (1 mL) was added HOAt (23.24 mg, 170.74 μmol, 23.88 μL, 1.5 eq), EDCI (43.64 mg, 227.65 μmol, 2 eq) and NMM (69.08 mg, 682.95 μmol, 75.09 μL, 6 eq). The mixture was stirred at 25° C. for 1 hr. The reaction mixture was quenched by water (0.5 mL) to get a mixture. The mixture was purified by reversed phase HPLC (ISCO®; 30 g SepaFlash® C18 Column, Eluent of 0˜95% (0.1% FA) water/MeCN @ 16 mL/min) and lyophilization to give I-236-16 (100 mg, 72.64 μmol, 63.82% yield, 98% purity), which as white solid. LCMS: R^{t}=0.568 min, [M+H]⁺=1348.9. ¹H NMR (400 MHZ, CHLOROFORM-d) 8=9.96-9.69 (m, 1H), 8.28 (d, J=6.4 Hz, 1H), 7.95-7.75 (m, 1H), 7.75-7.61 (m, 2H), 7.48-7.28 (m, 4H), 7.26-7.18 (m, 2H), 7.13-6.96 (m, 2H), 6.93-6.72 (m, 2H), 6.52 (br s, 1H), 5.22-5.00 (m, 2H), 4.76-4.66 (m, 1H), 4.65-4.45 (m, 2H), 4.44-4.33 (m, 2H), 4.23-4.06 (m, 1H), 4.06-3.91 (m, 1H), 3.91-3.76 (m, 3H), 3.75-3.54 (m, 7H), 3.53-3.32 (m, 4H), 3.21-2.43 (m, 15H), 2.38-1.90 (m, 10H), 1.90-1.79 (m, 4H), 1.70-1.51 (m, 5H), 1.51-1.40 (m, 18H), 1.31-1.06 (m, 5H).

### Step 14; Synthesis of I-236

To a solution of I-236-16 (90 mg, 66.71 μmol, 1 eq) in dioxane (1 mL) was added HCl/dioxane (4 M, 1 mL, 59.96 eq). The mixture was stirred at 25° C. for 1 hr. The reaction mixture was concentrated and quenched by NaHCO₃ (1 mL) and MeOH (2 mL) to get a mixture. The mixture was purified by reversed phase HPLC (ISCO®; 30 g SepaFlash® C18 Column, Eluent of 0˜95% (0.1% FA) water/MeCN @ 16 mL/min) and lyophilization to give product. I-236 (65 mg, 50.51 μmol, 75.71% yield, 100% purity, 3FA) as white solid. LCMS: R^{t}=0.445 min, [M+H]⁺=1148.7. SFC: R^{t}=4.416 min, 5.110 min. ¹H NMR (400 MHz, METHANOL-d4) δ=8.41 (s, 2H), 8.14 (d, J=2.0 Hz, 1H), 7.77 (br s, 1H), 7.74-7.66 (m, 1H), 7.58-7.40 (m, 3H), 7.35 (d, J=4.0 Hz, 4H), 7.14 (m, 1H), 7.08-6.93 (m, 2H), 6.63 (m, 1H), 5.15 (m, 1H), 4.99-4.91 (m, 1H), 4.64-4.43 (m, 3H), 4.10-3.96 (m, 3H), 3.96-3.86 (m, 1H), 3.85-3.57 (m, 10H), 3.57-3.43 (m, 3H), 3.42-3.33 (m, 2H), 3.29-3.05 (m, 5H), 2.93-2.68 (m, 2H), 2.28-2.16 (m, 2H), 2.13 (m, 1H), 2.09-1.97 (m, 4H), 1.96-1.74 (m, 10H), 1.73-1.55 (m, 6H), 1.55-1.40 (m, 1H), 1.38-1.18 (m, 3H), 1.17-1.02 (m, 2H). ¹⁹F NMR (377 MHz, METHANOL-d4) δ=−111.32 (s, 1F), −114.86 (s, 1F).

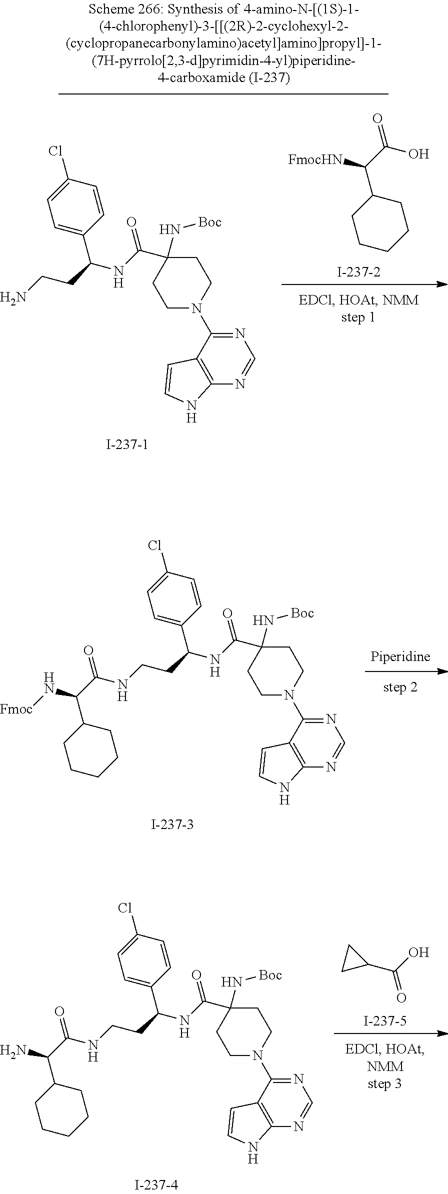

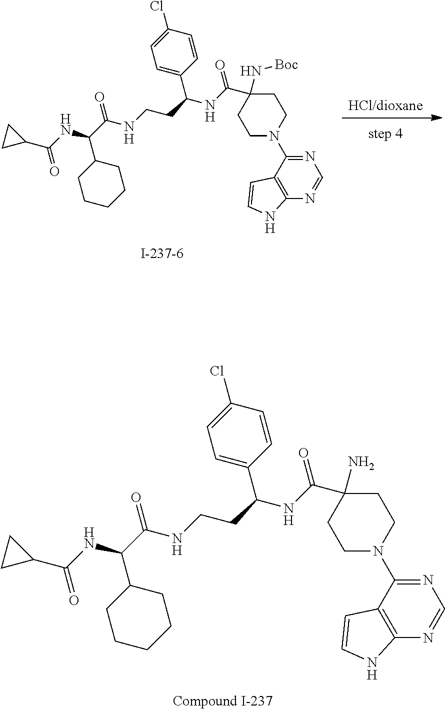 [SMILES: CC(C)(C)OC(=O)NC1(C(=O)N[C@@H](CCNC(=O)[C@H](NC(=O)C2CC2)C2CCCCC2)c2ccc(Cl)cc2)CCN(c2ncnc3[nH]ccc23)CC1 ; "HCl/dioxane" ; NC1(C(=O)N[C@@H](CCNC(=O)[C@H](NC(=O)C2CC2)C2CCCCC2)c2ccc(Cl)cc2)CCN(c2ncnc3[nH]ccc23)CC1]

### Step 1: Synthesis of I-237-3

To a solution of I-237-1 (50 mg, 94.69 μmol, 1 eq) in DMF (0.5 mL) was added I-237-2 (35.93 mg, 94.69 μmol, 35.93μ, 1 eq), EDCI (36.30 mg, 189.38 μmol, 2 eq), NMM (47.89 mg, 473.44 μmol, 52.05 ML, 5 eq) and HOAt (12.89 mg, 94.69 μmol, 13.25 ML, 1 eq). The mixture was stirred at 25° C. for 1 hr. The mixture was not worked up and used in next step. A solution of I-237-3 (80 mg, crude) in DMF (0.5 mL) as a yellow liquid. LCMS: R^{t}=0.782 min, [M+H]⁺=889.6.

### Step 2: Synthesis of I-237-4

To a solution of I-237-3 (80 mg, 89.94 μmol, 1 eq) in DMF (0.5 mL) was added PIPERIDINE (215.55 mg, 2.53 mmol, 0.25 mL, 28.15 eq). The mixture was stirred at 25° C. for 0.5 hr. The mixture was diluted with water (2 mL), extracted with EtOAc (5 mL*3). The organic layer concentrated under vacuum. The residue was purified by prep-TLC (SiO₂, DCM:MeOH=5:1). I-237-4 (40 mg, 59.95 μmol, 66.65% yield) as white solid was obtained. LCMS: R^{t}=0.615 min, [M+H]⁺=667.5. SFC: R^{t}=2.255 min.

### Step 3: Synthesis of I-237-6

To a solution of I-237-4 (40 mg, 59.95 μmol, 1 eq) in DMF (0.5 mL) was added I-237-5 (5.16 mg, 59.95 μmol, 4.74 μL, 1 eq), EDCI (22.98 mg, 119.90 μmol, 2 eq), NMM (30.32 mg, 299.74 μmol, 32.95 μL, 5 eq) and HOAt (8.16 mg, 59.95 μmol, 8.39 μL, 1 eq). The mixture was stirred at 25° C. for 1 hr. The mixture was diluted with water (2 mL), extracted with EtOAc (5 mL*3). The organic layer concentrated under vacuum. The residue was purified by prep-TLC (SiO₂, DCM:MeOH=5:1) and concentrated under vacuum. The residue was purified by Pre-HPLC (column: Waters Xbridge 150*25 mm*5 um; mobile phase: [water (NH₄HCO₃)-ACN]; gradient: 40%-70% B over 9 min) and dried by lyophilization. I-237-6 (13 mg, 17.68 μmol, 29.49% yield) as white solid was obtained. LCMS: R^{t}=0.636 min, [M+H]⁺=735.4. SFC: R^{t}=1.349 min, ee %=86.75%.

### Step 4: Synthesis of I-237

To a solution of I-237-6 (12 mg, 16.32 μmol, 1 eq) in DCM (50 μL) was added HCl/dioxane (2 M, 92.31 μL, 11.31 eq). The mixture was stirred at 25° C. for 1 hr. The mixture was concentrated under vacuum and dried by lyophilization to give I-237 (8 mg, 11.91 μmol, 72.98% yield, 100% purity, HCl) as white solid. LCMS: R^{t}=0.543 min, [M+H]⁺=635.5. SFC: R^{t}=1.928 min. NMR (400 MHZ, METHANOL-d4) δ=8.37 (s, 1H), 7.36 (d, J=3.6 Hz, 1H), 7.31 (s, 4H), 6.93 (d, J=3.6 Hz, 1H), 4.95-4.91 (m, 1H), 4.76-4.69 (m, 2H), 4.06 (d, J=7.6 Hz, 1H), 3.84-3.73 (m, 2H), 3.27-3.23 (m, 1H), 3.20-3.14 (m, 1H), 2.67-2.60 (m, 1H), 2.58-2.50 (m, 1H), 2.20-2.15 (m, 1H), 2.06-2.00 (m, 2H), 1.97-1.91 (m, 1H), 1.77 (br d, J=4.4 Hz, 3H), 1.74-1.71 (m, 1H), 1.71-1.68 (m, 1H), 1.68-1.60 (m, 2H), 1.34-1.28 (m, 2H), 1.25 (br d, J=10.4 Hz, 2H), 1.21-1.14 (m, 1H), 1.13-1.03 (m, 2H), 0.85-0.81 (m, 2H), 0.79-0.73 (m, 2H).

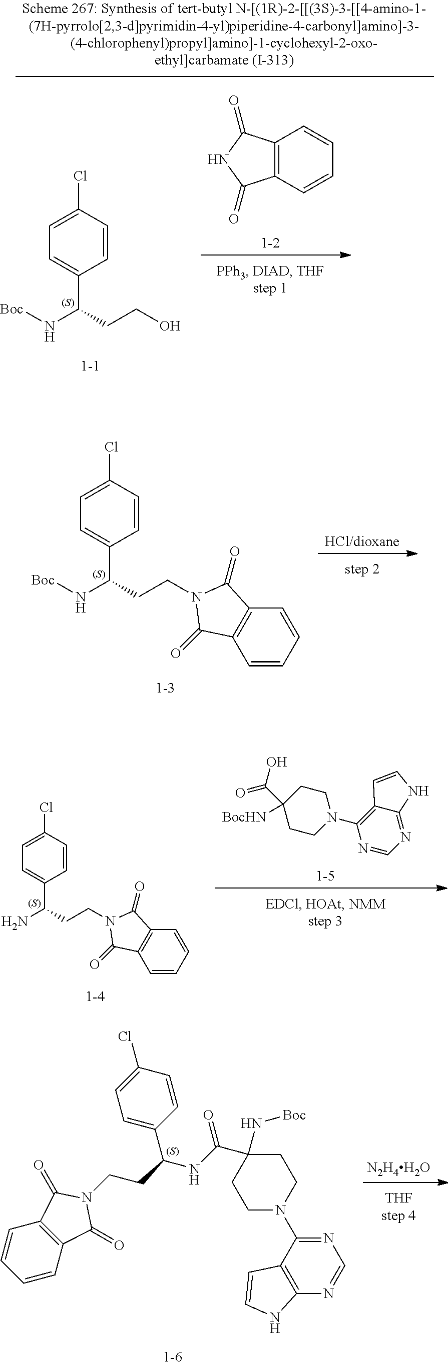

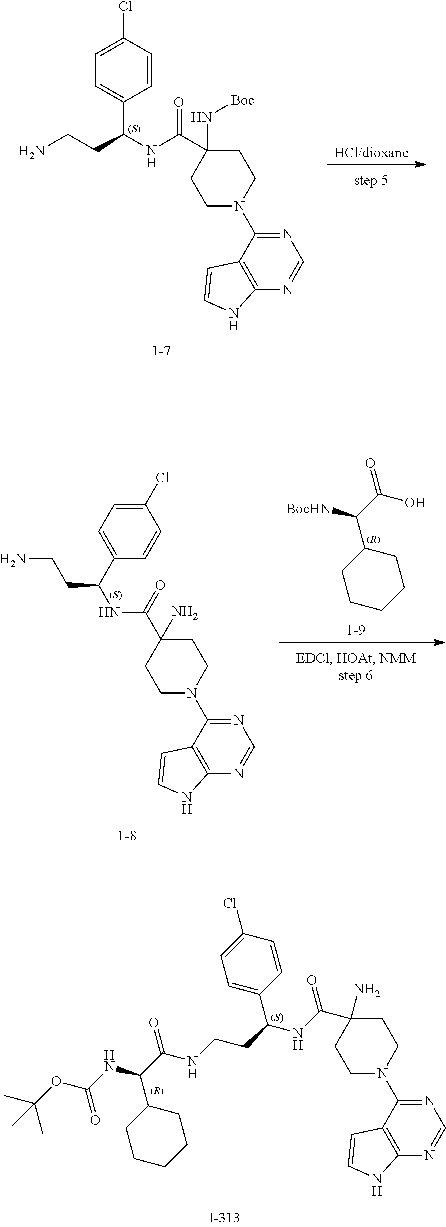

To a solution of 1-1 (1 g, 3.50 mmol, 1 eq), 1-2 (772.30 mg, 5.25 mmol, 1.5 eq) and PPh₃ (1.84 g, 7.00 mmol, 2 eq) in THF (10 mL) was added DIAD (1.42 g, 7.00 mmol, 1.36 mL, 2 eq) at 0° C. The mixture was stirred at 40° C. for 1 hr. The mixture was diluted with water (10 mL), extracted with EtOAc (10 mL*3) and washed with brine (5 mL). The organic layer was dried with anhydrous Na₂SO₄, filtered, and concentrated under vacuum. The residue was purified by column chromatography (SiO₂, Petroleum ether/Ethyl acetate=1/0 to 2/1) and concentrated under vacuum. 1-3 (1.6 g, crude) was obtained as a white solid. ¹H NMR (400 MHZ, DMSO-d₆) δ=11.33 (br s, 1H), 7.82 (s, 4H), 7.36-7.27 (m, 4H), 4.56-4.41 (m, 1H), 3.66-3.53 (m, 2H), 2.01-1.97 (m, 2H), 1.34 (s, 9H).

### Step 2: Synthesis of 1-4

To a solution of 1-3 (1.5 g, 3.62 mmol, 1 eq) in DCM (5 mL) was added HCl/dioxane (2 M, 10 mL, 5.53 eq). The mixture was stirred at 25° C. for 1 hr. The mixture was concentrated under vacuum to give 1-4 (1.2 g, crude, HCl) as white solid was obtained. LCMS: R^{t}=0.422 min, [M+H]⁺=314.9.

### Step 3: Synthesis of 1-6

To a solution of 1-4 (1.2 g, 3.42 mmol, 1 eq, HCl) in DMF (10 mL) was added 1-5 (1.23 g, 3.42 mmol, 1 eq), EDCI (1.31 g, 6.83 mmol, 2 eq), NMM (1.73 g, 17.08 mmol, 1.88 mL, 5 eq) and HOAt (465.03 mg, 3.42 mmol, 477.94 μL, 1 eq). The mixture was stirred at 25° C. for 2 hr. The reaction mixture was triturated with water (30 ml), filtered, and the filter cake was washed with water (10 mL*3), the filter cake was dried under vacuum. The filter cake which was dried under vacuum to give 1-6 (1.6 g, 2.43 mmol, 71.15% yield) as a white solid. LCMS: R^{t}=0.671 min, [M+H]⁺=658.4.

### Step 4: Synthesis of 1-7

To a solution of 1-6 (1.8 g, 2.73 mmol, 1 eq) in THF (18 mL) was added N₂H₄·H₂O (900.00 mg, 17.62 mmol, 872.09 μL, 98% purity, 6.44 eq). The mixture was stirred at 25° C. for 16 hr. The reaction mixture was added 10 mL H₂O at 25° C., extracted with EA 60 mL (20 mL*3), then filtered, and concentrated under reduced pressure to give a residue, aqueous phase was quenched by HCl solution (1 M). The residue was purified by prep-TLC (SiO₂, DCM/MeOH=3/1) and concentrated under vacuum. 1-7 (1.1 g. 2.08 mmol, 76.17% yield) as white oil. LCMS: R^{t}=0.659 min, [M+H]⁺=528.3. SFC: R^{t}=1.933 min.

### Step 5: Synthesis of 1-8

To a solution of 1-7 (100 mg, 189.38 μmol, 1 eq) in DCM (0.5 mL) was added HCl/dioxane (4 M, 0.5 mL, 10.56 eq). The mixture was stirred at 25° C. for 0.5 hr. The mixture was concentrated under vacuum to give 1-8 (80 mg, crude, HCl) as yellow oil. LCMS: R^{t}=0.318 min, [M+H]⁺=428.1.

### Step 6: Synthesis of I-313

To a solution of 1-8 (40 mg, 86.13 μmol, 1 eq, HCl) in DMF (0.5 mL) was added 1-9 (24.05 mg, 93.47 μmol, 1 eq), EDCI (35.84 mg, 186.95 μmol, 2 eq), NMM (47.27 mg, 467.37 μmol, 51.38 μL, 5 eq) and HOAt (12.72 mg, 93.47 μmol, 13.08 μL, 1 eq). The mixture was stirred at 25° C. for 1 hr. The mixture was diluted with water (2 mL), extracted with EtOAc (5 mL*3). The organic layer concentrated under vacuum. The residue was purified by Prep-HPLC (column: Waters Xbridge 150*25 mm*5 um; mobile phase: [water (NH₄HCO₃)-ACN]; gradient: 40%-70% B over 9 min) and dried by lyophilization. I-313 (20 mg, 29.05 μmol, 31.08% yield, 96.92% purity) was obtained as a white solid. LCMS: R^{t}=0.637 min, [M+H]⁺=667.4. SFC: R^{t}=1.947 min. ¹H NMR (400 MHZ, DMSO-d₆) δ=11.64 (br s, 1H), 8.34 (br d, J=8.0 Hz. 1H), 8.11 (s. 1H), 7.92-7.89 (m, 1H), 7.37-7.32 (m, 4H), 7.18-7.11 (m, 1H), 6.62-6.52 (m, 2H). 4.82-4.73 (m, 1H), 4.41-4.30 (m, 2H), 3.75-3.89 (m, 1H), 3.59-3.50 (m, 2H), 3.01 (br d, J=5.6 Hz. 2H), 2.25 (br s, 1H), 2.00-1.85 (m, 3H), 1.83-1.76 (m, 1H), 1.62 (br s, 3H), 1.56-1.39 (m, 5H), 1.35 (s. 10H), 1.14-0.92 (m, 5H).

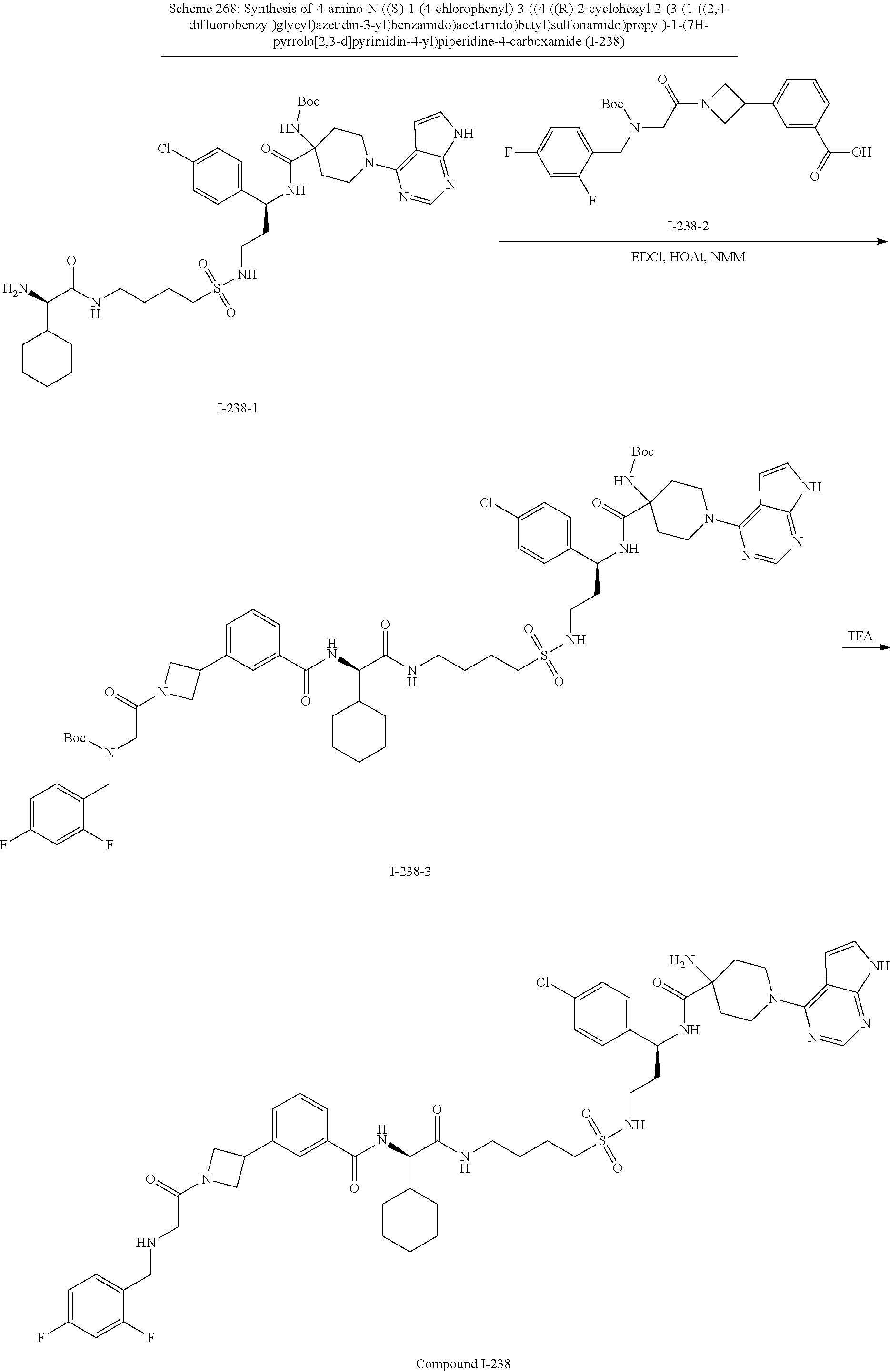 [SMILES: CC(C)(C)OC(=O)NC1(C(=O)N[C@@H](CCNS(=O)(=O)CCCCNC(=O)[C@H](N)C2CCCCC2)c2ccc(Cl)cc2)CCN(c2ncnc3[nH]ccc23)CC1 ; CC(C)(C)OC(=O)N(CC(=O)N1CC(c2cccc(C(=O)O)c2)C1)Cc1ccc(F)cc1F ; "TFA" ; CC(C)(C)OC(=O)NC1(C(=O)N[C@@H](CCNS(=O)(=O)CCCCNC(=O)[C@H](NC(=O)c2cccc(C3CN(C(=O)CN(Cc4ccc(F)cc4F)C(=O)OC(C)(C)C)C3)c2)C2CCCCC2)c2ccc(Cl)cc2)CCN(c2ncnc3[nH]ccc23)CC1 ; NC1(C(=O)N[C@@H](CCNS(=O)(=O)CCCCNC(=O)[C@H](NC(=O)c2cccc(C3CN(C(=O)CNCc4ccc(F)cc4F)C3)c2)C2CCCCC2)c2ccc(Cl)cc2)CCN(c2ncnc3[nH]ccc23)CC1]

### Step 1: Synthesis of I-238-3

A mixture of I-238-1 (50 mg, 62.31 μmol, 1 eq). I-238-2 (28.69 mg, 62.31 μmol, 1 eq) and HOAt (8.48 mg, 62.31 μmol, 8.72 μL, 1 eq) in DMF (1 mL) was added EDCI (23.89 mg, 124.62 μmol, 2 eq) and NMM (31.51 mg, 311.56 μmol, 34.25 μL, 5 eq). The mixture was stirred at 25° C. for 1 hrs. The mixture was poured into water (6 mL) and filtered. The filter cake was concentrated under vacuum. The residue was purified by Pre-HPLC (column: Waters Xbridge 150*25 mm*5 um; mobile phase: [water (NH4HCO3)-ACN]; gradient: 45%-75% B over 10 min) and concentrated under vacuum to give I-238-3 (40 mg, 32.13 μmol, 51.57% yield) as white solid. LCMS: R^{t}=2.214 min, [M−H]⁺=1244.9. SFC: R^{t}=1.009 min.

### Step 2: Synthesis of I-238

To a solution of I-238-3 (40 mg, 32.13 μmol, 1 eq) in DCM (1 mL) was added TFA (153.50 mg, 1.35 mmol, 0.1 mL, 41.90 eq). The mixture was stirred at 25° C. for 2 hr. The mixture was adjusted to pH=8 with NH₃·H₂O and concentrated under vacuum. The residue was purified by Pre-HPLC (column: Waters Xbridge 150*25 mm*5 um; mobile phase: [water (NH4HCO3)-ACN]; gradient: 35%-65% B over 9 min) and dried by lyophilization. I-238 (16.71 mg, 14.30 μmol, 44.49% yield, 99.129% purity, TFA) as white solid was obtained, and. LCMS: R^{t}=0.432 min, [M+H]⁺=1044.6. SFC: R^{t}=0.570, 1.327 min. ¹H NMR (400 MHZ, CHLOROFORM-d) δ=10.63 (br s, 1H), 8.37-8.18 (m, 2H), 7.77 (br d, J=2.8 Hz, 1H), 7.67 (br d, J=4.0 Hz, 1H), 7.53 (br s, 1H), 7.37 (br d, J=2.8 Hz, 3H), 7.31-7.27 (m, 2H), 7.25-7.18 (m, 2H), 7.07 (d, J=3.2 Hz, 1H), 6.89-6.72 (m, 2H), 6.47 (br s, 1H), 6.33 (br s, 1H), 5.11 (br t, J=8.0 Hz, 1H), 4.66-4.51 (m, 1H), 4.48-4.34 (m, 3H), 4.13 (br s, 1H), 4.07-3.97 (m, 1H), 3.86 (br d, J=5.4 Hz, 3H), 3.67-3.46 (m, 2H), 3.35-3.26 (m, 2H), 3.24-2.83 (m, 5H), 2.43-2.24 (m, 4H), 2.23-2.13 (m, 2H), 2.10-2.00 (m, 2H), 1.96-1.94 (m, 1H), 1.86 (br d, J=10.0 Hz, 2H), 1.79-1.78 (m, 2H), 1.71 (br d, J=9.6 Hz, 3H), 1.66-1.43 (m, 5H), 1.41-0.94 (m, 6H). ¹⁹F NMR (376 MHz, CHLOROFORM-d) δ=−75.48 (br s, 1F), −111.13-−111.60 (m, 1F), −114.59 (br d, J=26.9 Hz, 1F).

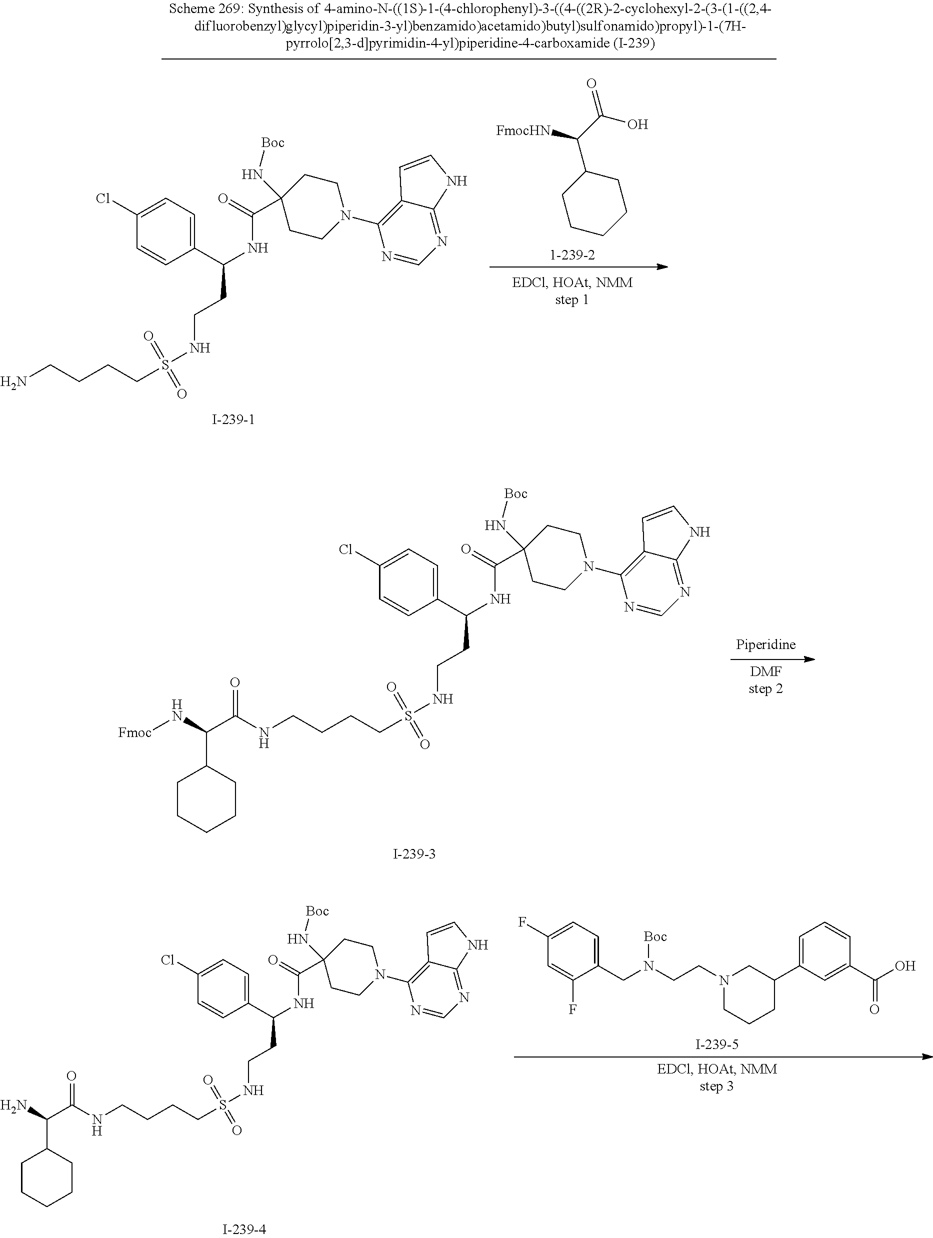

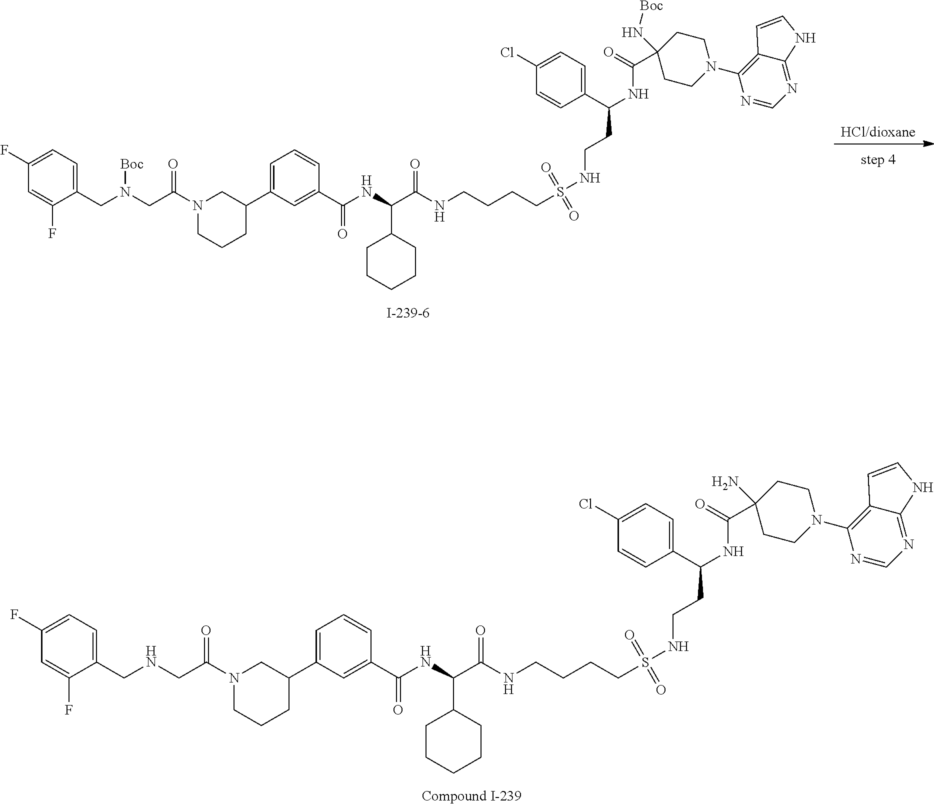 [SMILES: CC(C)(C)OC(=O)NC1(C(=O)N[C@@H](CCNS(=O)(=O)CCCCNC(=O)[C@H](NC(=O)c2cccc(C3CCCN(C(=O)CN(Cc4ccc(F)cc4F)C(=O)OC(C)(C)C)C3)c2)C2CCCCC2)c2ccc(Cl)cc2)CCN(c2ncnc3[nH]ccc23)CC1 ; "HCl/dioxane" ; NC1(C(=O)N[C@@H](CCNS(=O)(=O)CCCCNC(=O)[C@H](NC(=O)c2cccc(C3CCCN(C(=O)CNCc4ccc(F)cc4F)C3)c2)C2CCCCC2)c2ccc(Cl)cc2)CCN(c2ncnc3[nH]ccc23)CC1]

### Step 1: Synthesis of I-239-3

To a solution of I-239-2 (240.29 mg, 633.27 μmol, 240.29 μL, 1 eq) in DMF (4 mL) was added HOAt (86.19 mg, 633.27 μmol, 1 eq), EDCI (242.80 mg, 1.27 mmol, 2 eq), NMM (320.26 mg, 3.17 mmol, 348.11 μL, 5 eq) and I-239-1 (0.42 g, 633.27 μmol, 1 eq). The mixture was stirred at 25° C. for 1 h. as a major peak. The reaction mixture was poured onto water (30 ml), filtered, and the filter cake was washed with water (10 mL*3), the filter cake was dried under vacuum to give a residue. The crude product was purified by reverse-phase HPLC (0.1% NH₃·H₂O) to give I-239-3 (0.36 g, 351.34 μmol, 55.48% yield) as a white solid. LCMS: R^{t}=0.757 min, [M+H]⁺=1024.6. SFC: R^{t}=2.017 min. ¹H NMR (400 MHZ, DMSO-d6) δ=11.66 (br s, 1H), 8.12 (s, 1H), 8.06 (br d, J=8.3 Hz, 1H), 7.96 (br t, J=5.1 Hz, 1H), 7.88 (d, J=7.5 Hz, 2H), 7.73 (t, J=6.9 Hz, 2H), 7.43-7.38 (m, 2H), 7.35-7.28 (m, 7H), 7.19-7.11 (m, 1H), 7.00 (br d, J=5.5 Hz, 2H), 6.58 (br d, J=1.3 Hz, 1H), 4.94-4.85 (m, 1H), 4.30-4.17 (m, 5H), 3.79 (br t, J=8.3 Hz, 1H), 3.57 (br d, J=8.3 Hz, 2H), 3.10-2.87 (m, 6H), 1.99-1.77 (m, 6H), 1.67-1.48 (m, 10H), 1.39 (br s, 9H), 1.13-0.91 (m, 5H).

### Step 2: Synthesis of I-239-4

To a solution of I-239-3 (0.31 g, 302.54 μmol, 1 eq) in DMF (3 mL) was added Piperidine (257.60 mg, 3.03 mmol, 298.77 μL, 10 eq). The mixture was stirred at 25° C. for 20 min. The reaction mixture was quenched by H₂O (10 mL), extracted with EA (5 mL*3). The combined organic layers were washed with brine (5 mL*3), dried over anhydrous Na₂SO₄, filtered, and concentrated under reduced pressure to give a residue. The residue was purified by reverse-phase HPLC (0.1% NH₃·H₂O) to give I-239-4 (200 mg, 249.24 μmol, 82.38% yield) as a white solid. LCMS: R^{t}=0.619 min, [M+H]⁺=802.5. SFC: R^{t}=2.096 min.

### Step 3: Synthesis of I-239-6

To a solution of I-239-4 (40 mg, 49.85 μmol, 1 eq) in DMF (0.4 mL) was added HOAt (6.78 mg, 49.85 μmol, 1 eq), NMM (25.21 mg, 249.24 μmol, 27.40 μL, 5 eq), I-239-5 (24.35 mg, 49.85 μmol, 1 eq) and EDCI (19.11 mg, 99.70 μmol, 2 eq). The mixture was stirred at 25° C. for 1 hr. The reaction mixture was poured onto water (1.5 ml), filtered, and the filter cake was washed with water (1 mL*3), the filter cake was dried under vacuum to give a residue. The crude product was purified by reverse-phase HPLC (0.1% NH₃·H₂O) to give I-239-6 (35 mg, 27.21 μmol, 54.58% yield, 98.952% purity) as a white solid. LCMS: R^{t}=0.801 min, [M+H]⁺=1272.8. SFC: R^{t}=1.844 min.

### Step 4: Synthesis of I-239

To a solution of I-239-6 (35 mg, 27.50 μmol, 1 eq) in DCM (0.3 mL) was added HCl/dioxane (4 M, 0.07 mL, 10.18 eq). The mixture was stirred at 25° C. for 1 h. The mixture was concentrated under vacuum to give a residue. The residue was diluted with MeOH (0.5 ml) and H₂O (20 ml) and freeze-dried to give I-239 (12.37 mg, 10.70 μmol, 38.93% yield, 95.974% purity, HCl) as a white solid. LCMS: R^{t}=1.938 min, [M+H]⁺=1072.7. SFC: R^{t}=2.012 min, 2.213 min. ¹H NMR (400 MHz, METHANOL-d4) 8=8.34 (s, 1H), 7.87-7.76 (m, 1H), 7.76-7.70 (m, 1H), 7.69-7.60 (m, 1H), 7.53-7.40 (m, 2H), 7.38-7.29 (m, 5H), 7.16-7.06 (m, 2H), 6.91 (d, J=3.6 Hz, 1H), 5.08-5.10 (m, 1H), 4.66-4.41 (m, 3H), 4.36-4.14 (m, 5H), 3.85-3.7 (m, 2H), 3.73 (br d, J=13.2 Hz, 1H), 3.27-3.21 (m, 2H), 3.13-3.02 (m, 4H), 2.93-2.73 (m, 2H), 2.69-2.57 (m, 2H), 2.21-2.02 (m, 4H), 1.94-1.64 (m, 14H), 1.33-1.09 (m, 6H). ¹⁹F NMR (377 MHz, METHANOL-d4) δ=−108.42 (td, J=6.7, 13.5 Hz, 1F), −112.62-−112.88 (m, 1F).

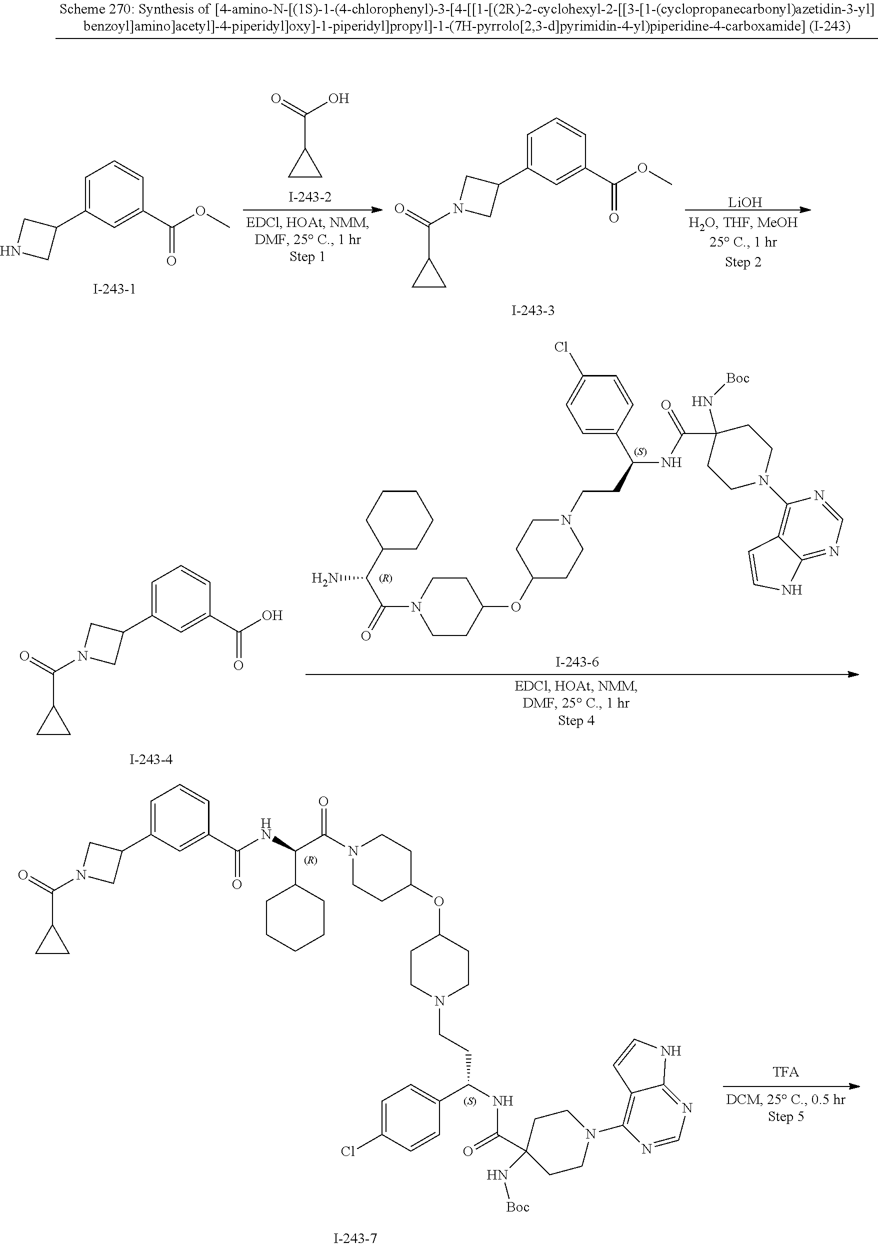

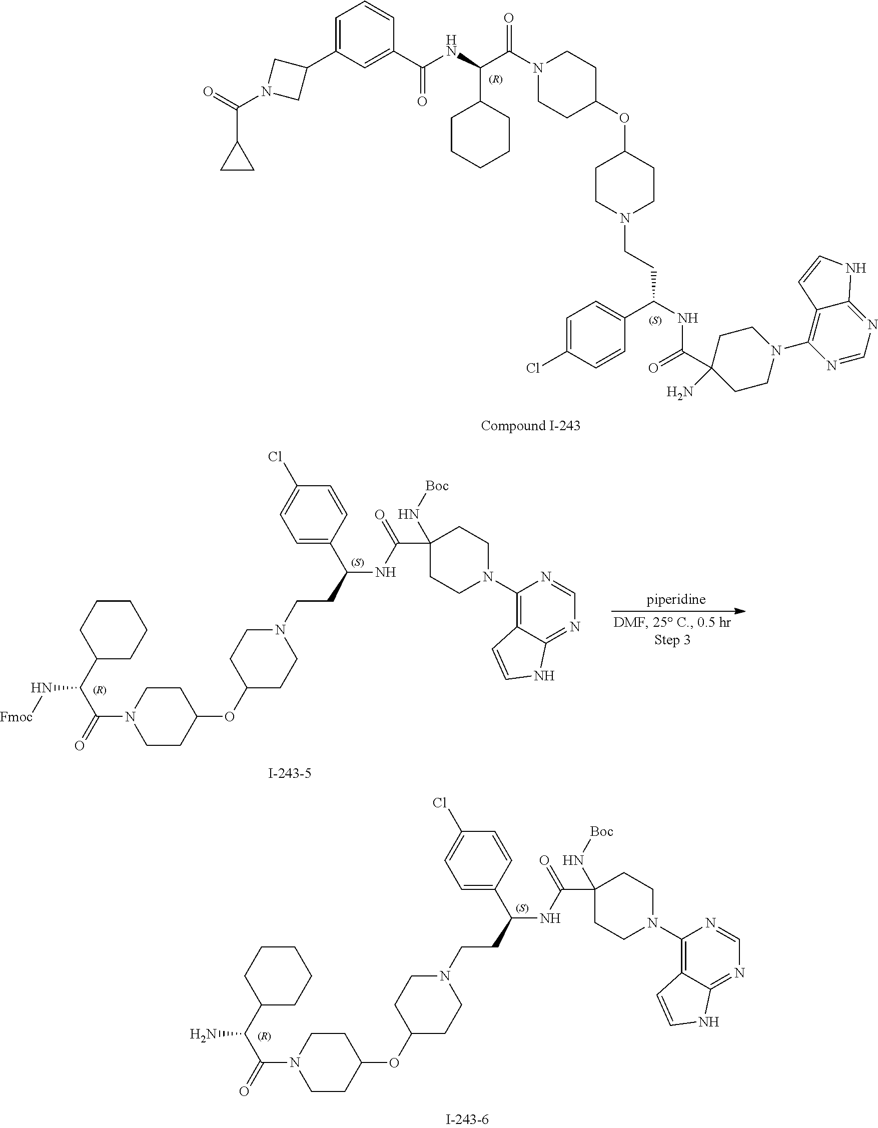

### Step 1: Synthesis of I-243-3.

To a solution of I-243-1 (200 mg, 655.20 μmol, 1 eq, TFA) in DMF (2 mL) was added NMM (331.36 mg, 3.28 mmol, 360.17 μL, 5 eq) at 25° C., the mixture was stirred at 25° C. for 10 min. Then I-243-2 (56.41 mg, 655.20 μmol, 51.84 μL, 1 eq), EDCI (628.01 mg, 3.28 mmol, 5 eq) and HOAt (178.36 mg, 1.31 mmol, 2 eq) were added at 25° C. The resulting mixture was stirred at 25° C. for 1 h. The reaction mixture was washed with water (2 mL) and extracted with DCM (2 mL*3), the combined organic phase was dried by Na₂SO₄, concentrated under reduced pressure to give a residue. The residue was purified by flash silica gel chromatography (ISCO®; 12 g SepaFlash® Silica Flash Column, Eluent of 0˜50% Ethyl acetate/Petroleum ether @30 mL/min) and the eluent was concentrated to give I-243-3 (200 mg, 325.29 μmol, 49.65% yield, 42.174% purity) as a white solid. LCMS (Method D): R^{t}=0.378 min, [M+H]⁺=260.0.

### Step 2: Synthesis of I-243-4.

To a solution of I-243-3 (150 mg, 578.48 μmol, 1 eq) in MeOH (0.5 mL), THF (0.5 mL), H₂O (0.5 mL) was added LiOH·H₂O (72.83 mg, 1.74 mmol, 3 eq). The mixture was stirred at 25° C. for 1 h. The pH was adjusted to 5 with 1M HCl, the reaction mixture was extracted with DCM (2 mL*3), the combined organic phase was dried by Na₂SO₄, concentrated under reduced pressure to give I-243-4 (145 mg, 571.20 μmol, 98.74% yield, 96.620% purity) as a colorless oil. LCMS (Method D): R^{t}=0.301 min, [M+H]⁺=246.1.

### Step 3: Synthesis of I-243-6.

To a mixture of I-243-5 (100 mg, 94.63 μmol, 1 eq) in DMF (1 mL) was added piperidine (16.12 mg, 189.26 μmol, 18.69 μL, 2 eq) at 25° C., the mixture was stirred at 25° C. for 0.5 h. The reaction mixture was diluted with H₂O (1 mL) and extracted with DCM (1 mL×3), the combined organic layers were dried over Na₂SO₄, filtered, and concentrated to give I-243-6 (80 mg, crude) as a yellow oil. LCMS (Method D):

R^{t}=0.284 min, [M+H]⁺=834.6.

### Step 4: Synthesis of I-243-7.

To a mixture of I-243-6 (72 mg, 86.28 μmol, 1 eq) in DMF (1 mL) was added I-243-4 (21.16 mg, 86.28 μmol, 1 eq), EDCI (49.62 mg, 258.84 μmol, 3 eq), HOAt (11.74 mg, 86.28 μmol, 12.07 μL, 1 eq) and NMM (43.64 mg, 431.40 μmol, 47.43 μL, 5 eq) at 25° C., the mixture was stirred at 25° C. for 1 h. The reaction mixture was diluted with H₂O (1 mL) and extracted with DCM (1 mL×3), the combined organic layers were dried over Na₂SO₄, filtered, and concentrated to give a residue. The residue was purified by column chromatography (SiO₂, EA/MeOH=1/0 to 1/1) and concentrated to give I-243-7 (70 mg, 65.93 μmol, 76.41% yield, N/A purity) as a yellow solid. LCMS (Method D): R^{t}=0.439 min, [M+H]⁺=1061.8.

### Step 5: Synthesis of I-243.

To a mixture of I-243-7 (60 mg, 56.51 μmol, 1 eq) in DCM (0.6 mL) was added TFA (307.00 mg, 2.69 mmol, 0.2 mL, 47.64 eq) at 25° C., the mixture was stirred at 25° C. for 0.5 h. The reaction mixture was concentrated to give a residue. The residue was purified by prep-HPLC (column: CD01-Phenomenex luna C18 150*25*10 um; mobile phase: [water (TFA)-ACN]; gradient: 20%-50% B over 10 min) and lyophilized to give I-243 (17.7 mg, 15.81 μmol, 27.97% yield, 96.058% purity, TFA) as a white solid. LCMS (Method D): R^{t}=0.383 min, [M+H]⁺=961.7. SFC: R^{t}=0.560 min. ¹H NMR (400 MHZ, METHANOL-d4) δ=8.32 (d, J=2.4 Hz, 1H), 7.86 (s, 1H), 7.75 (d, J=6.8 Hz, 1H), 7.59-7.54 (m, 1H), 7.52-7.45 (m, 1H), 7.36 (d, J=4.0 Hz, 4H), 7.31 (t, J=3.6 Hz, 1H), 6.84 (t, J=3.6 Hz, 1H), 5.04-4.96 (m, 1H), 4.96-4.88 (m, 2H), 4.77 (t, J=8.4 Hz, 1H), 4.73-4.60 (m, 2H), 4.48-4.36 (m, 2H), 4.11-3.97 (m, 3H), 3.96-3.88 (m, 1H), 3.83-3.66 (m, 4H), 3.64-3.47 (m, 2H), 3.36 (s, 1H), 3.25-3.13 (m, 3H), 3.13-2.92 (m, 2H), 2.66-2.47 (m, 2H), 2.41-2.29 (m, 1H), 2.28-2.19 (m, 1H), 2.11 (d, J=15.2 Hz, 1H), 2.07-1.98 (m, 2H), 1.98-1.85 (m, 5H), 1.82-1.74 (m, 2H), 1.74-1.58 (m, 5H), 1.57-1.46 (m, 1H), 1.34-1.22 (m, 3H), 1.17-1.04 (m, 2H), 0.93-0.80 (m, 4H). ¹⁹F NMR (376 MHz, METHANOL-d4) δ=−77.074 (br s, 1F), —F.

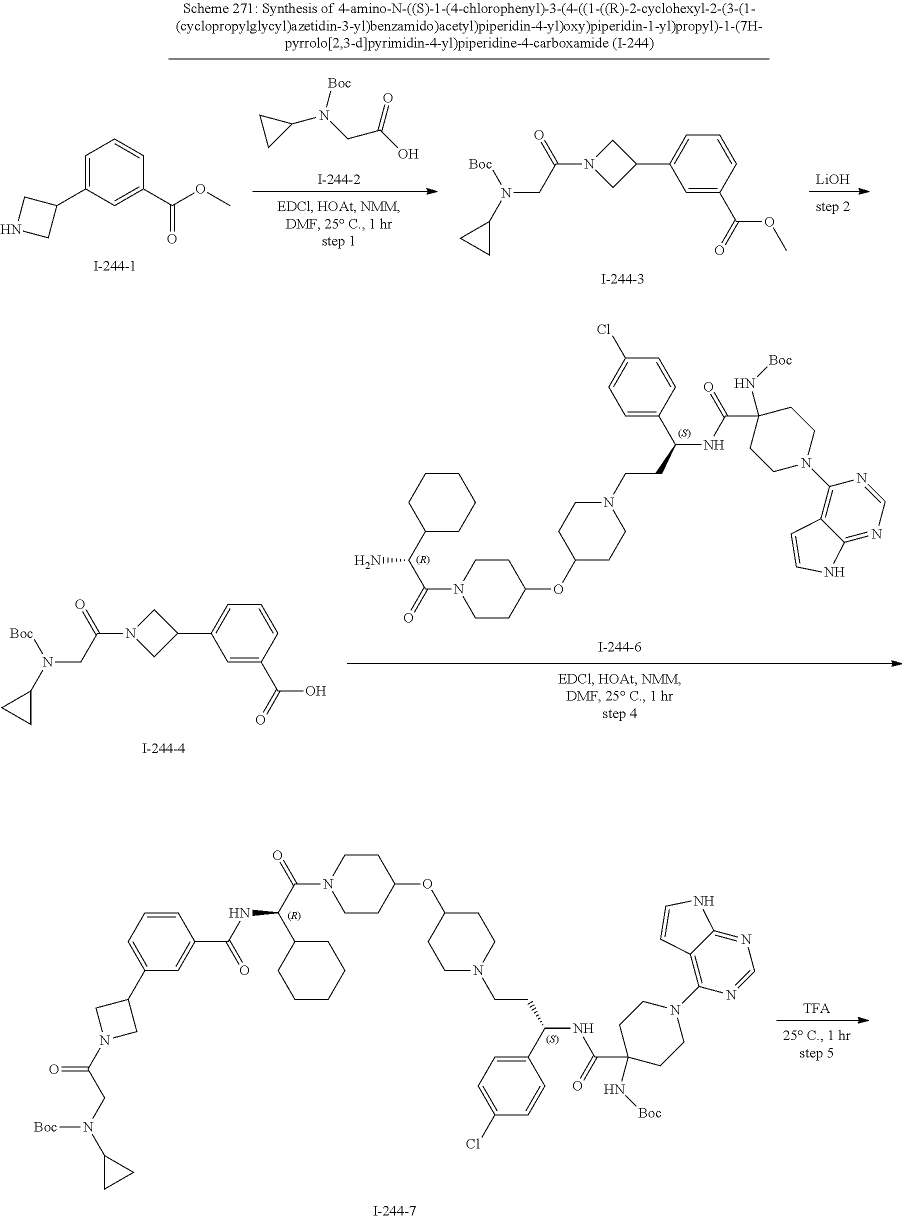

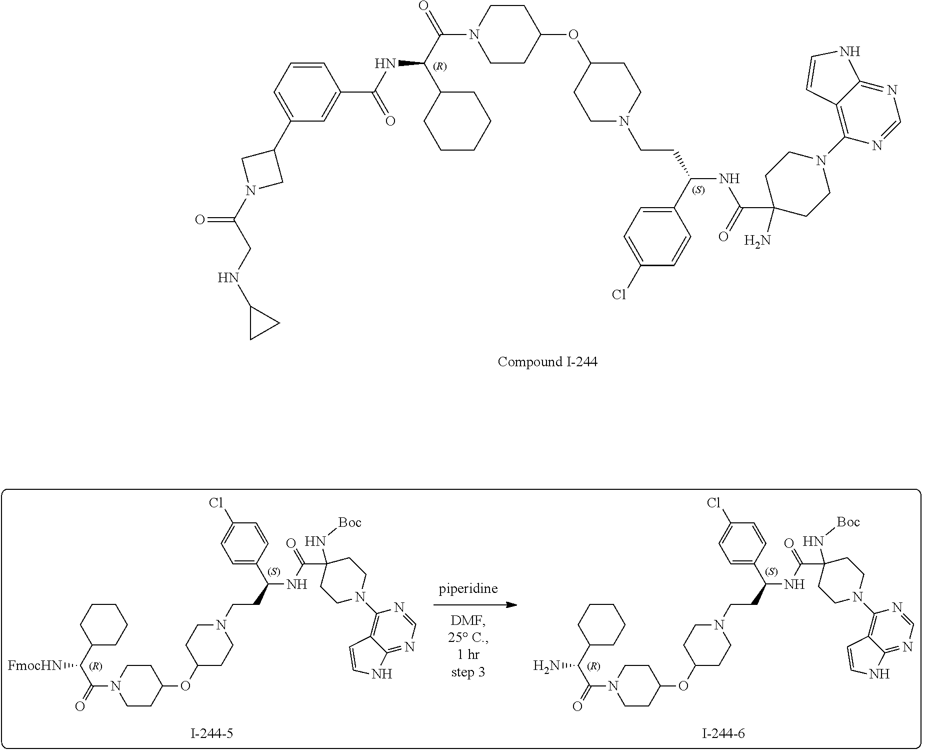

### Step 1: Synthesis of I-244-3.

To a solution of I-244-1 (200 mg, 655.20 μmol, 1 eq, TFA) in DMF (2 mL) was added NMM (331.36 mg, 3.28 mmol, 360.17 μL, 5 eq) at 25° C., the mixture was stirred at 25° C. for 10 min. Then I-244-2 (141.03 mg, 655.20 μmol, 1 eq), EDCI (628.01 mg, 3.28 mmol, 5 eq) and HOAt (178.36 mg, 1.31 mmol, 183.31 μL, 2 eq) were added at 25° C. The resulting mixture was stirred at 25° C. for 1 hr. There mixture was washed with water (4 mL) and extracted with DCM (4 mL*3), the combined organic phase was dried by Na₂SO₄, concentrated under reduced pressure to give a residue. The residue was purified by flash silica gel chromatography (ISCO®; 20 g SepaFlash® Silica Flash Column, Eluent of 0˜50% Ethyl acetate/Petroleum ether @40 mL/min) and the eluent was concentrated to give I-244-3 (405 mg, 871.29 μmol, 66.49% yield, 83.570% purity) as a white solid. LCMS (Method D): R^{t}=0.461 min, [M+Na]⁺=411.0.

### Step 2: Synthesis of I-244-4.

To a solution of I-244-3 (390 mg, 1.00 mmol, 1 eq) in MeOH (1.2 mL), THF (1.2 mL), H₂O (1.2 mL) was added LiOH·H₂O (126.39 mg, 3.01 mmol, 3 eq). The mixture was stirred at 25° C. for 1 hr. The pH was adjusted to 5 with 1M HCl, the reaction mixture was extracted with DCM (4 mL*3), the combined organic phase was dried by Na₂SO₄, concentrated under reduced pressure to give I-244-4 (400 mg, crude) as a colorless oil. LCMS (Method D): R^{t}=0.379 min, [M+H]⁺=375.1.

### Step 3: Synthesis of I-244-6.

To a solution of I-244-5 (100 mg, 94.63 μmol, 1 eq) in DMF (1 mL) was added piperidine (16.12 mg, 189.26 μmol, 18.69 μL, 2 eq). The mixture was stirred at 25° C. for 1 hr. The reaction mixture was added H₂O (2 mL) and then extracted with DCM (2 mL*3), the combined organic phase was washed with dried by Na₂SO₄, filtered, and concentrated to give I-244-6 (80 mg, crude) as a white solid. LCMS (Method D): R^{t}=0.297 min, [M+H]⁺=834.5.

### Step 4: Synthesis of I-244-7.

To a solution of I-244-4 (70 mg, 83.88 μmol, 1 eq), I-244-6 (31.41 mg, 83.88 μmol, 1 eq) in DMF (0.7 mL) was added EDCI (32.16 mg, 167.77 μmol, 2 eq) and HOAt (11.42 mg, 83.88 μmol, 1 eq) and NMM (42.42 mg, 419.42 μmol, 46.11 μL, 5 eq). The mixture was stirred at 25° C. for 1 hr. The reaction mixture was added H₂O (2 mL) and then extracted with DCM (2 mL*3), the combined organic phase was washed with dried by Na₂SO₄, filtered, and concentrated. The residue was purified by flash silica gel chromatography (ISCO®; 12 g SepaFlash® Silica Flash Column, Eluent of 0˜100% Ethyl acetate/Petroleum ether gradient @12 mL/min) and the eluent was concentrated to give I-244-7 (80 mg, 67.18 μmol, 80.08% yield, N/A purity) as a white solid. LCMS (Method D): R^{t}=0.396 min, [M+H]⁺=1190.8.

### Step 5: Synthesis of I-244.

To a solution of I-244-7 (70 mg, 58.78 μmol, 1 eq) in DCM (0.9 mL) was added TFA (460.50 mg, 4.04 mmol, 0.3 mL, 68.71 eq). The mixture was stirred at 25° C. for 1 hr. The reaction mixture was concentrated under reduced pressure to give a residue. The residue was purified by prep-HPLC (column: CD04-Welch Ultimate C18 150*25*7 um; mobile phase: [water (TFA)-ACN]; gradient: 8%-38% B over 11 min) the eluent was concentrated to remove ACN and lyophilized to get I-244 (7.91 mg, 7.16 μmol, 12.18% yield, 100% purity, TFA) as a white solid. LCMS (Method D): R^{t}=0.294 min, [M+H]⁺=990.6. SFC: R^{t}=1.739 min. ¹H NMR (400 MHZ, METHANOL-d4) δ=8.32 (d, J=2.0 Hz, 1H), 7.84 (d, J=5.2 Hz, 1H), 7.77 (d, J=7.6 Hz, 1H), 7.63-7.56 (m, 1H), 7.54-7.47 (m, 1H), 7.37 (d, J=3.6 Hz, 4H), 7.31 (t, J=3.2 Hz, 1H), 6.84 (t, J=3.6 Hz, 1H), 5.06-4.98 (m, 1H), 4.94 (d, J=8.4 Hz, 1H), 4.71-4.56 (m, 3H), 4.56-4.48 (m, 1H), 4.38-4.29 (m, 1H), 4.21-4.06 (m, 2H), 4.06-3.96 (m, 3H), 3.92 (s, 1H), 3.87-3.69 (m, 4H), 3.66-3.49 (m, 2H), 3.42-3.35 (m, 1H), 3.28-3.16 (m, 3H), 3.15-2.95 (m, 2H), 2.81 (s, 1H), 2.69-2.49 (m, 2H), 2.40-2.07 (m, 4H), 2.07-1.92 (m, 4H), 1.92-1.83 (m, 3H), 1.83-1.60 (m, 6H), 1.59-1.48 (m, 1H), 1.34-1.21 (m, 3H), 1.17-1.04 (m, 2H), 0.96-0.88 (m, 4H), ¹⁹F NMR (377 MHz, METHANOL-d4), δ=−76.986, —F.

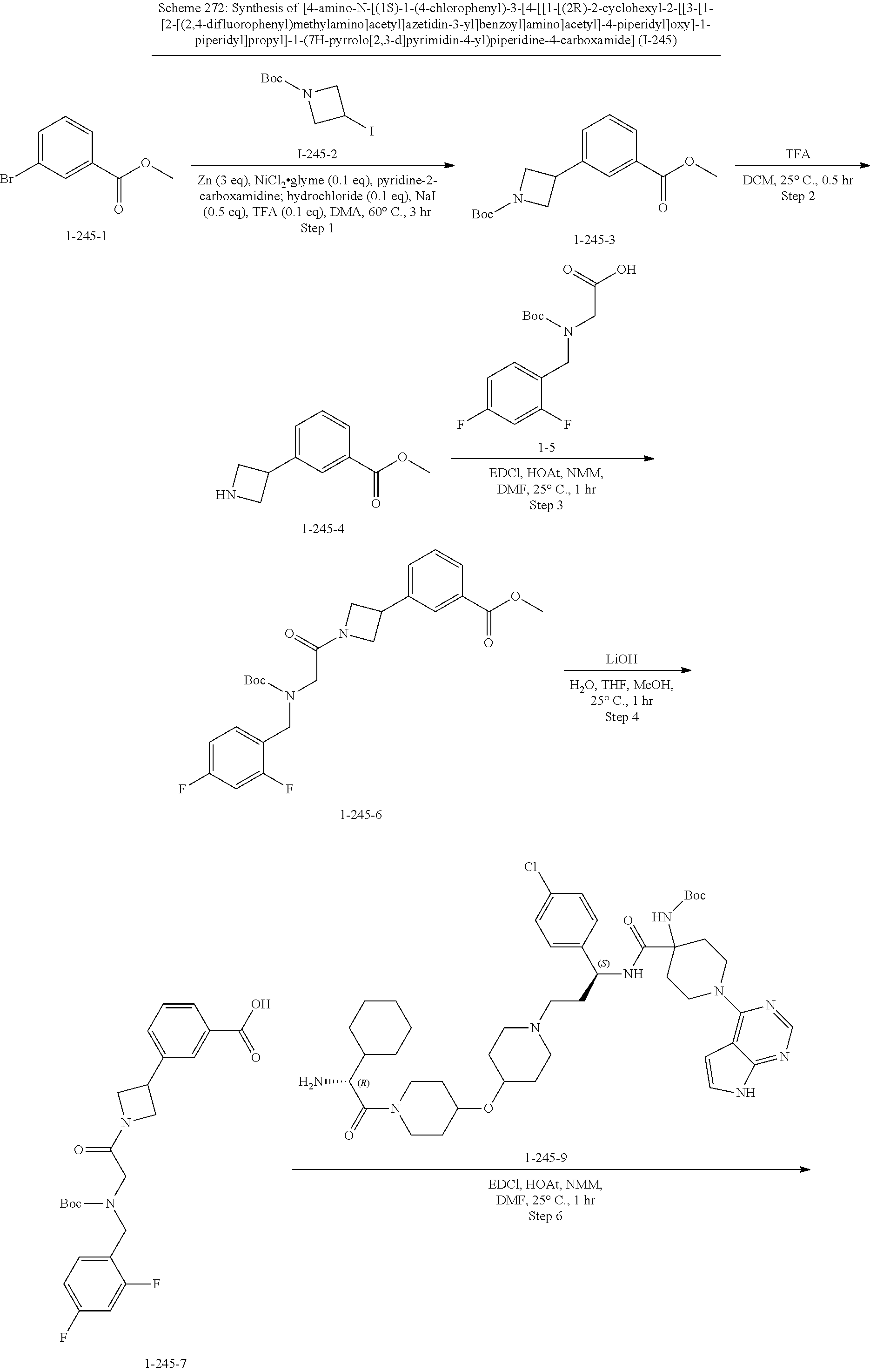

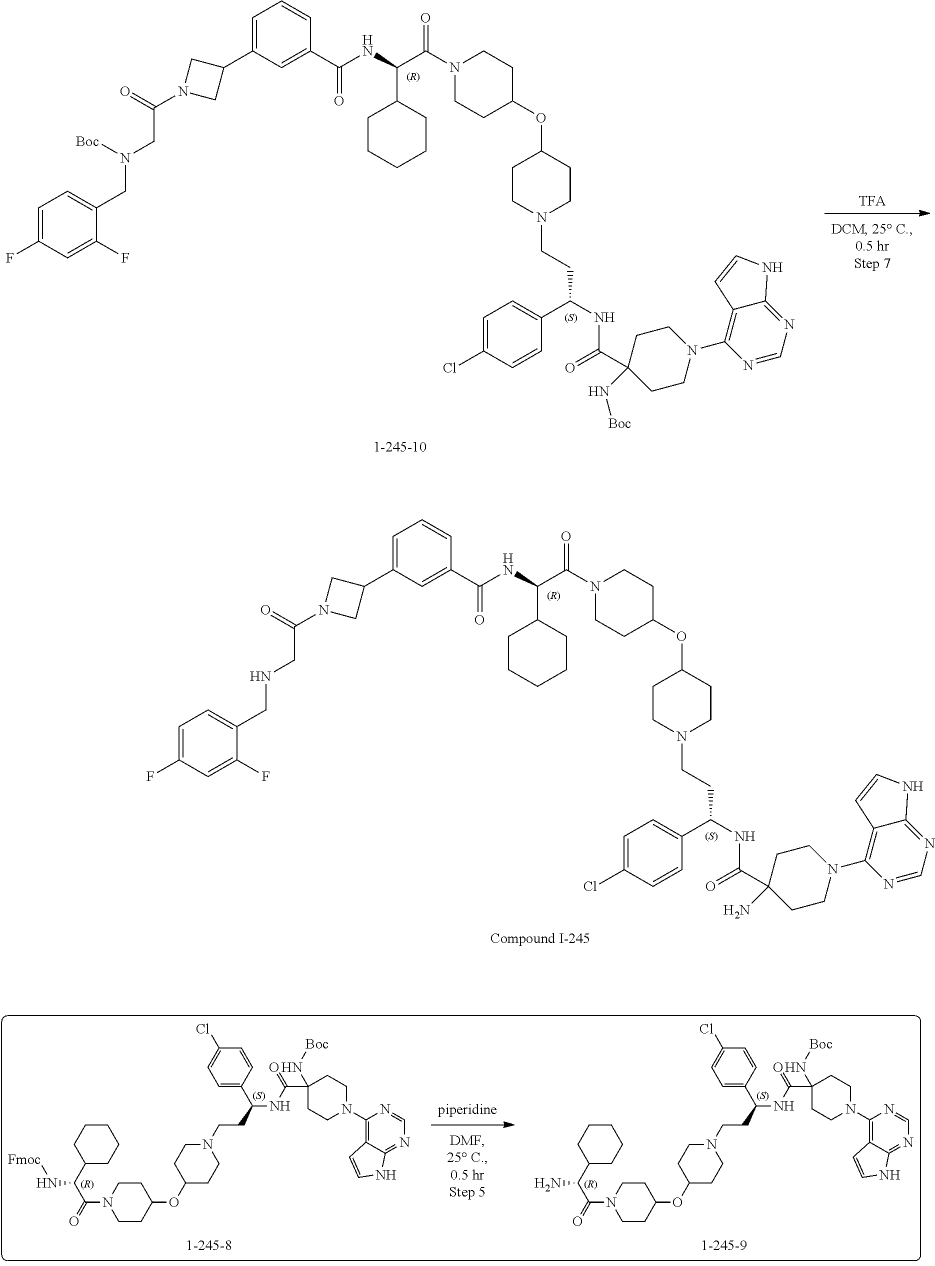

### Step 1: Synthesis of I-245-3.

A mixture of I-245-1 (500 mg, 2.33 mmol, 1 eq), I-245-2 (1.32 g, 4.65 mmol, 2 eq), NiCl₂·glyme (510.87 mg, 2.33 mmol, 1 eq), NaI (34.85 mg, 232.51 μmol, 0.1 eq), TFA (132.56 mg, 1.16 mmol, 86.36 μL, 0.5 eq) and pyridine-2-carboxamidine; hydrochloride (36.64 mg, 232.51 μmol, 0.1 eq) in DMA (5 mL) was degassed and purged with N₂ for 3 times, then Manganese (255.47 mg, 4.65 mmol, 253.19 μL, 2 eq) was added to the mixture and stirred at 60° C. for 2 h under N₂ atmosphere. The reaction mixture was filtered, and diluted with H₂O (10 mL), extracted with DCM (5 mL×3), the combined organic layers were dried over Na₂SO₄, filtered, and concentrated to give a residue. The residue was purified by column chromatography (SiO₂, PE/EA=1/0 to 8/2) and concentrated to give I-245-3 (120 mg, 411.89 μmol, 17.71% yield, N/A purity) as a yellow oil. LCMS (Method D): R^{t}=0.394 min, [M-55]⁺=236.1. ¹H NMR (400 MHz, METHANOL-d4) δ=8.00 (s, 1H), 7.92 (d, J=8.0 Hz, 1H), 7.60 (d, J=8.0 Hz, 1H), 7.52-7.45 (m, 1H), 4.37 (t, J=8.0 Hz, 2H), 3.97-3.93 (m, 2H), 3.92 (s, 3H), 3.90-3.84 (m, 1H), 1.47 (s, 9H).

### Step 2: Synthesis of I-245-4.

To a mixture of I-245-3 (100 mg, 343.24 μmol, 1 eq) in DCM (1 mL) was added TFA (307.00 mg, 2.69 mmol, 0.2 mL, 7.84 eq), the mixture was stirred at 25° C. for 0.5 h. The reaction mixture was concentrated to give I-245-4 (100 mg, crude, TFA) as a yellow gum. LCMS (Method D): R^{t}=0.125 min, [M+H]⁺=192.2.

### Step 3: Synthesis of I-245-6.

To a mixture of I-245-4 (100 mg, 327.60 μmol, 1 eq. TFA) in DMF (1 mL) was added I-245-5 (98.70 mg, 327.60 μmol, 1 eq), EDCI (188.40 mg, 982.80 μmol, 3 eq), HOAt (44.59 mg, 327.60 μmol, 1 eq) and NMM (165.68 mg, 1.64 mmol, 180.09 μL, 5 eq) at 25° C., the mixture was stirred at 25° C. for 1 h. The reaction mixture was diluted with H₂O (2 mL), extracted with DCM (2 mL×3), the combined organic layers were dried over Na₂SO₄, filtered, and concentrated to give a residue. The residue was purified by column chromatography (SiO₂, PE/EA=1/0 to 1/1) and concentrated to give I-245-6 (160 mg, 305.67 μmol, 93.31% yield, 90.649% purity) as a yellow gun. LCMS (Method D): R^{t}=0.428 min, [M+H]⁺=475.1.

### Step 4: Synthesis of I-245-7.

To a mixture of I-245-6 (140 mg, 295.05 μmol, 1 eq) in H₂O (0.5 mL), THF (0.5 mL) and MeOH (0.5 mL) was added LiOH·H₂O (37.14 mg, 885.15 μmol, 3 eq) at 25° C., the mixture was stirred at 25° C. for 1 h. The reaction mixture was diluted with H₂O (2 mL) and extracted with DCM (2 mL×3), the combined organic layers were dried over Na₂SO₄, filtered, and concentrated to give I-245-7 (150 mg, crude) as a yellow gum. LCMS (Method D): R^{t}=0.399 min, [M+Na]⁺=483.1.

### Step 5: Synthesis of I-245-9.

To a mixture of I-245-8 (100 mg, 94.63 μmol, 1 eq) in DMF (1 mL) was added piperidine (16.12 mg, 189.26 μmol, 18.69 μL, 2 eq) at 25° C., the mixture was stirred at 25° C. for 0.5 h. The reaction mixture was diluted with H₂O (1 mL) and extracted with DCM (1 mL×3), the combined organic layers were dried over Na₂SO₄, filtered, and concentrated to give I-245-9 (80 mg, crude) as a yellow oil. LCMS (Method D): R^{t}=0.286 min, [M+H]⁺=834.6.

### Step 6: Synthesis of I-245-10.

To a mixture of I-245-9 (72 mg, 86.28 μmol, 1 eq) in DMF (1 mL) was added I-245-7 (39.73 mg, 86.28 μmol, 1 eq), EDCI (49.62 mg, 258.84 μmol, 3 eq), HOAt (11.74 mg, 86.28 μmol, 1 eq) and NMM (43.64 mg, 431.40 μmol, 47.43 μL, 5 eq) at 25° C., the mixture was stirred at 25° C. for 1 h. The reaction mixture was diluted with H₂O (1 mL) and extracted with DCM (1 mL×3), the combined organic layers were dried over Na₂SO₄, filtered, and concentrated to give a residue. The residue was purified by column chromatography (SiO₂, EA/MeOH=1/0 to 1/1) and concentrated to give I-245-10 (70 mg, 54.82 μmol, 63.54% yield, N/A purity) as a yellow solid. LCMS (Method D): R^{t}=0.504 min, [M+H]⁺=1277.0.

### Step 7: Synthesis of I-245

To a mixture of I-245-10 (60 mg, 46.99 μmol, 1 eq) in DCM (0.6 mL) was added TFA (307.00 mg, 2.69 mmol, 0.2 mL, 57.30 eq) at 25° C., the mixture was stirred at 25° C. for 0.5 h. The reaction mixture was concentrated to give a residue. The residue was purified by prep-HPLC (column: CD01-Phenomenex luna C18 150*25*10 um; mobile phase: [water (TFA)-ACN]; gradient: 29%-59% B over 10 min) and lyophilized to give I-245 (9.93 mg, 8.34 μmol, 17.75% yield, 100.00% purity, TFA) as a white solid. LCMS (Method D): R^{t}=0.314 min, [M+H]⁺=1076.5. SFC: R^{t}=0.623 min. ¹H NMR (400 MHZ, METHANOL-d4) 8=8.30 (d, J=2.8 Hz, 1H), 7.89-7.82 (m, 1H), 7.77 (d, J=7.6 Hz, 1H), 7.65-7.54 (m, 2H), 7.54-7.47 (m, 1H), 7.37 (d, J=3.6 Hz, 4H), 7.29 (t, J=3.2 Hz, 1H), 7.22-7.03 (m, 2H), 6.80 (t, J=3.6 Hz, 1H), 5.05-4.98 (m, 1H), 4.94 (d, J=8.8 Hz, 1H), 4.70-4.59 (m, 3H), 4.57-4.48 (m, 1H), 4.33 (s, 2H), 4.32-4.25 (m, 1H), 4.20-4.06 (m, 2H), 4.01 (s, 1H), 3.96-3.88 (m, 3H), 3.87-3.79 (m, 1H), 3.78-3.68 (m, 3H), 3.67-3.57 (m, 1H), 3.56-3.44 (m, 2H), 3.42-3.36 (m, 1H), 3.26-3.12 (m, 3H), 3.12-2.95 (m, 2H), 2.64-2.46 (m, 2H), 2.37-2.37 (m, 1H), 2.37-2.20 (m, 2H), 2.11 (d, J=14.4 Hz, 1H), 2.05-2.01 (m, 1H), 2.00-1.92 (m, 3H), 1.91-1.82 (m, 3H), 1.81-1.75 (m, 2H), 1.74-1.58 (m, 4H), 1.58-1.46 (m, 1H), 1.33-1.23 (m, 3H), 1.21-1.08 (m, 2H). ¹⁹F NMR (376 MHz, METHANOL-d4) δ=−77.020 (br s, 1F), −108.207 (br s, 1F), −112.913 (br s, 1F), —F.

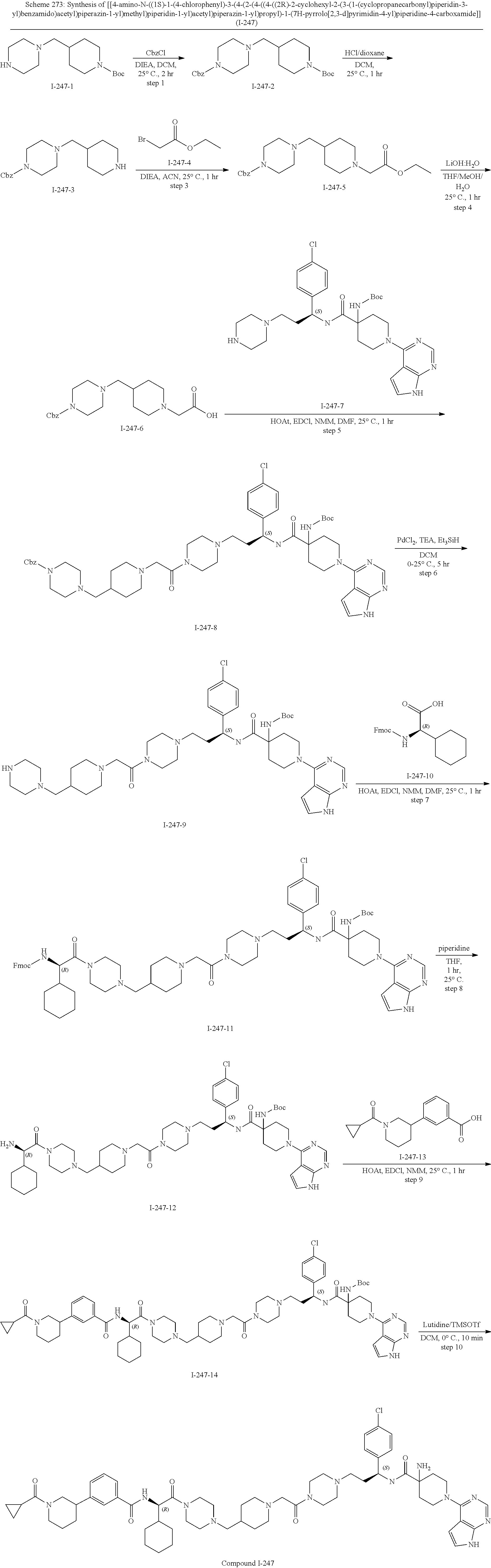

### Step 1: Synthesis of I-247-2

To a solution of I-247-1 (10 g, 35.28 mmol, 1 eq) in DCM (10 mL) was added CbzCl (9.03 g, 52.93 mmol, 7.56 mL, 1.5 eq) and DIEA (13.68 g, 105.85 mmol, 18.44 mL, 3 eq). The mixture was stirred at 25° C. for 2 hr. The mixture was concentrated under reduced pressure to give a residue. The residue was purified by flash silica gel chromatography (ISCO®; 120 g SepaFlash® Silica Flash Column, Eluent of 0˜100% PE/EA @ 100 mL/min, PE/EA=0:1, Rf=0.4) and the eluent was concentrated under reduced pressure to give I-247-2 (16 g, 30.66 mmol, 86.88% yield, 80% purity) as a yellow oil. LCMS (Method D): R^{t}=0.362 min, [M+H]⁺=418.1. ¹H NMR (400 MHZ, METHANOL-d4) δ=7.37-7.28 (m, 5H), 5.11 (s, 2H), 4.05 (d, J=13.2 Hz, 2H), 3.49 (s, 4H), 2.74 (s, 2H), 2.38 (s, 4H), 2.20 (d, J=6.8 Hz, 2H), 2.01 (s, 1H), 1.75 (d, J=10.8 Hz, 2H), 1.45 (s, 9H), 1.10-0.97 (m, 2H).

### Step 2: Synthesis of I-247-3

To a solution of I-247-2 (14 g, 33.53 mmol, 1 eq) in DCM (20 mL) was added HCl/dioxane (2 M, 140.00 mL, 8.35 eq. The mixture was stirred at 25° C. for 1 hr. The mixture was concentrated under reduced pressure to give I-247-3 (14 g, crude) as a white solid.

### Step 3: Synthesis of I-247-5

To a solution of I-247-3 (1 g, 2.83 mmol, 1 eq, HCl) in ACN (10 mL) was added DIEA (1.10 g, 8.48 mmol, 1.48 mL, 3 eq) and I-247-4 (566.29 mg, 3.39 mmol, 375.27 μL, 1.2 eq). The mixture was stirred at 25° C. for 1 hr. The mixture was washed with water (10 mL) and extract with EA (10 mL*3). The combined organic layers dried over sodium sulfate, filtered, and concentrated under reduced pressure to give I-247-5 (1.2 g, crude) as a yellow oil. LCMS (Method D): R^{t}=0.143 min, [M+H]⁺=404.3.

### Step 4: Synthesis of I-247-6

To a solution of I-247-5 (1.2 g, 2.97 mmol, 1 eq) in THF (4 mL), H₂O (4 mL), MeOH (4 mL) was added LiOH·H₂O (374.38 mg, 8.92 mmol, 3 eq). The mixture was stirred at 25° C. for 1 hr. The pH was acidified to 5˜6 with 1N HCl, and the mixture was washed with water (10 mL) and extract with (CH₃Cl/IPA=3:1) (8 mL*3). The combined organic layers dried over sodium sulfate, filtered, and concentrated under reduced pressure to give I-247-6 (1.1 g. 2.93 mmol, 98.52% yield) as a yellow oil. LCMS (Method D): R^{t}=0.206 min, [M+H]⁺=376.3.

### Step 5: Synthesis of I-247-8

To a solution of I-247-6 (471.57 mg, 1.26 mmol, 3.0 eq) in DMF (2.5 mL) was added HOAt (56.98 mg, 418.66 μmol, 1 eq), EDCI (240.77 mg, 1.26 mmol, 3 eq), NMM (211.73 mg, 2.09 mmol, 230.14 μL, 5 eq) and I-247-7 (250 mg, 418.66 μmol, 1 eq). The mixture was stirred at 25° C. for 1 hr. The mixture was washed with water (3 mL) and extract with EA (2 mL*3). The combined organic layers dried over sodium sulfate, filtered, and concentrated under reduced pressure to give a residue. The residue was purified by flash silica gel chromatography (ISCO®; 20 g SepaFlash® Silica Flash Column, Eluent of 0˜35% EA/MeOH gradient @ 50 mL/min) and then the eluent was concentrated in vacuo to give I-247-8 (360 mg, 371.96 μmol, 88.85% yield, 98.63% purity) as white solid. LCMS (Method D): R^{t}=0.264 min, [M+H]⁺=954.5. SFC: R^{t}=1.705 min.

### Step 6: Synthesis of I-247-9

To a solution of I-247-8 (360 mg, 377.12 μmol, 1 eq) in DCM (3 mL) was added PdCl2 (20.06 mg, 113.14 μmol, 0.3 eq) and TEA (76.32 mg, 754.25 μmol, 104.98 μL, 2 eq). The reaction mixture was degassed with N₂. Then a solution of Et₃SiH (219.25 mg, 1.89 mmol, 301.17 μL, 5 eq) in DCM (0.6 mL) was added dropwise at 10° C. reaction mixture was stirred at 10-25° C. for 5 hr. The mixture was filtered, and the filtrate was concentrated in vacuum. Then the mixture diluted with NaHCO₃ 5 mL and extracted with CH₂Cl₂ 10 mL (5 mL*2). The combined organic layers were dried over Na₂SO₄, filtered, and concentrated under reduced pressure to give I-247-9 (235 mg, 286.42 μmol, 75.95% yield) as white solid. LCMS (Method D): R^{t}=0.244 min, [M+H]⁺=820.3.

### Step 7: Synthesis of I-247-11

To a solution of I-247-10 (77.08 mg, 203.14 μmol, 77.08 μL, 1 eq) in DMF (2 mL) was added HOAt (27.65 mg, 203.14 μmol, 1 eq), EDCI (116.82 mg, 609.41 μmol, 3 eq), NMM (102.73 mg, 1.02 mmol, 111.67 μL, 5 eq) and I-247-9 (200 mg, 243.76 μmol, 1.2 eq). The mixture was stirred at 25° C. for 1 hr. The mixture was washed with water (3 mL) and extract with EA (2 mL+3). The combined organic layers dried over sodium sulfate, filtered, and concentrated under reduced pressure to give a residue. The residue was purified by reversed phase column (FA condition), the eluent was concentrated under reduced pressure to remove ACN and lyophilized to give I-247-11 (70 mg, 59.23 μmol, 29.16% yield) as white solid. LCMS (Method D): R^{t}=0.345 min, [M+H]⁺=1181.7.

### Step 8: Synthesis of I-247-12

To a solution of I-247-11 (70 mg, 59.23 μmol, 1 eq) in THF (0.7 mL) was added PIPERIDINE (0.09 mL). The mixture was stirred at 25° C. for 1 hr. The mixture was washed with water (1 mL) and extract with EA (2 mL*3). The combined organic layers dried over sodium sulfate, filtered, and concentrated under reduced pressure to give a residue. The residue was purified by reversed phase column (Neutral condition), the eluent was concentrated under reduced pressure to remove ACN and lyophilized to give I-247-12 (50 mg, 51.78 μmol, 87.43% yield, 99.39% purity) as white solid. LCMS (Method D): R^{t}=0.250 min, [M+H]⁺=595.6.

### Step 9: Synthesis of I-247-14

To a solution of I-247-13 (8.54 mg, 31.26 μmol, 1 eq) in DMF (0.3 mL) was added HOAt (4.25 mg, 31.26 μmol, 4.37 μL, 1 eq), EDCI (17.98 mg, 93.78 μmol, 3 eq), NMM (15.81 mg, 156.31 μmol, 17.18 μL, 5 eq) and I-247-12 (30 mg, 31.26 μmol, 1 eq). The mixture was stirred at 25° C. for 1 hr. The mixture was washed with water (1 mL) and extract with EA (2 mL*3). The combined organic layers dried over sodium sulfate, filtered, and concentrated under reduced pressure to give a residue. The residue was purified by reversed phase column (FA condition), the eluent was concentrated under reduced pressure to remove ACN and lyophilized to give I-247-14 (50 mg, crude) as a white solid. LCMS (Method D): R^{t}=0.324 min, [M+H]⁺=1214.6. SFC: R^{t}=4.825 min, 5.852 min.

### Step 10: Synthesis of I-247

To a solution of I-247-14 (40 mg, 32.92 μmol, 1 eq) in DCM (0.4 mL) was added 2,6-dimethylpyridine (1.06 mg, 9.88 μmol, 1.15 μL, 0.3 eq) and TMSOTf (10.98 mg, 49.38 μmol, 8.92 μL, 1.5 eq). The mixture was stirred at 0° C. for 10 min. The pH was basified to 7˜8 with NH₃·H₂O, the mixture was concentrated under reduced pressure to give a residue. The residue was purified by reversed phase column (NH₃·H₂O condition), The residue was purified by reversed phase column (FA condition), the eluent was concentrated under reduced pressure to remove ACN and lyophilized to give I-247 (10.57 mg, 8.88 μmol, 26.98% yield, 97.557% purity, FA) as a white solid. LCMS (Method D): R^{t}=0.300 min, [M+H]⁺=1114.4. SFC: R^{t}=2.802 min, 3.417 min. ¹H NMR (400 MHz, METHANOL-d4) δ=8.53 (s, 1H), 8.12 (s, 1H), 7.79-7.67 (m, 2H), 7.53-7.42 (m, 2H), 7.33 (s, 4H), 7.12 (d, J=3.6 Hz, 1H), 6.62 (d, J=3.6 Hz, 1H), 4.98 (m, 1H), 4.61-4.53 (m, 2H), 4.52-4.43 (m, 2H), 4.42-4.36 (m, 1H), 3.88-3.82 (m, 1H), 3.70 (s, 2H), 3.65-3.60 (m, 5H), 3.54-3.48 (m, 1H), 3.15-3.12 (m, 1H), 3.00-2.92 (m, 2H), 2.77-2.72 (m, 1H), 2.53-2.47 (m, 3H), 2.45 (d, J=5.2 Hz, 2H), 2.40-2.33 (m, 4H), 2.23 (s, 1H), 2.21 (s, 2H), 2.19-2.14 (m, 2H), 2.12 (d, J=4.4 Hz, 1H), 2.08-2.03 (m, 2H), 2.00-1.95 (m, 2H), 1.93-1.85 (m, 4H), 1.84-1.72 (m, 6H), 1.72-1.66 (m, 3H), 1.64-1.52 (m, 4H), 1.36-1.18 (m, 7H), 1.15-1.07 (m, 2H), 0.89 (d, J=4.8 Hz, 2H), 0.85-0.77 (m, 2H).

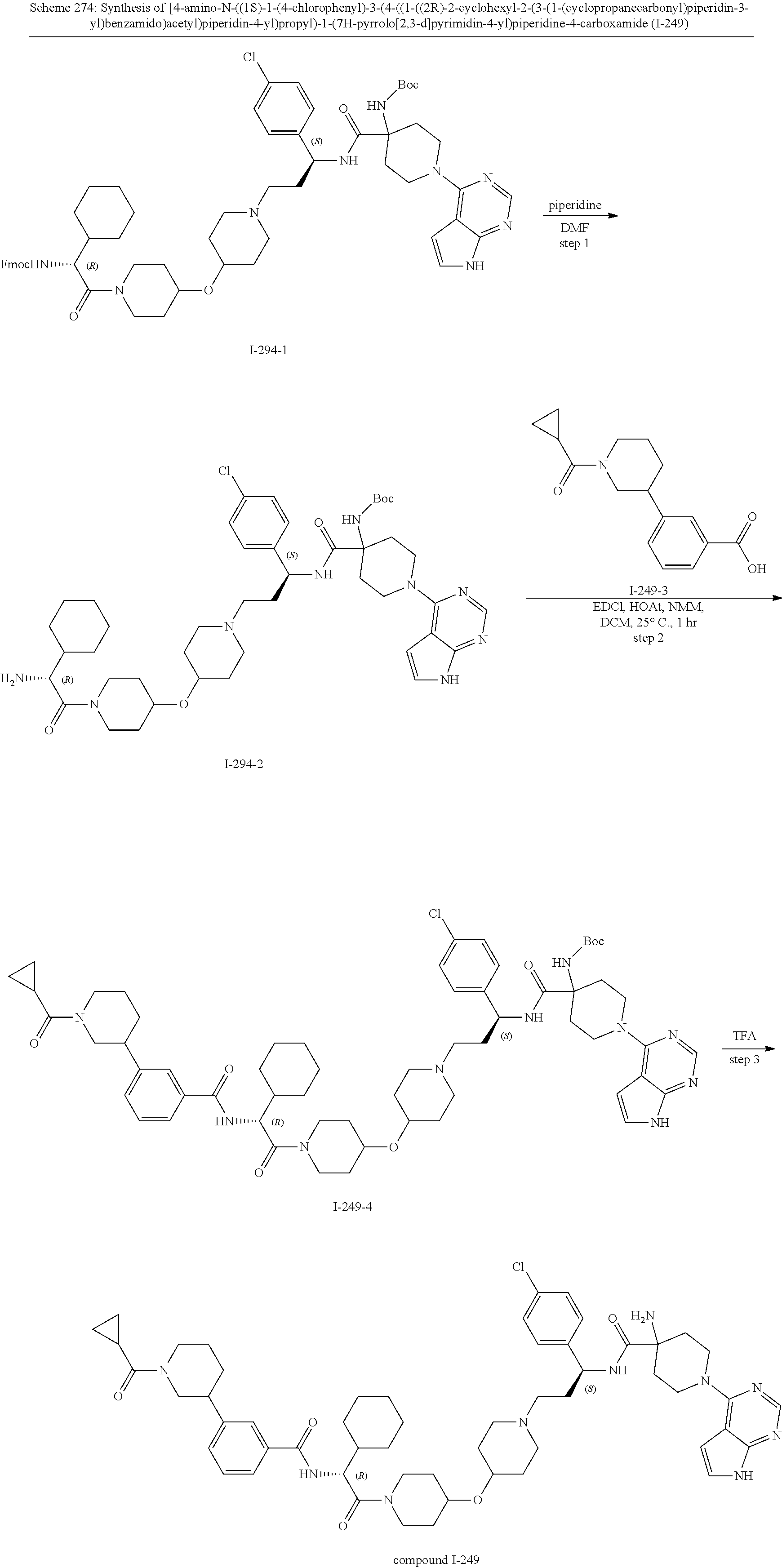

### Step 1: Synthesis of I-249-2

To a solution of I-249-1 (80 mg, 75.71 μmol, 1 eq) in DMF (1 mL) was added piperidine (19.34 mg, 227.12 μmol, 22.43 μL, 3 eq). The mixture was stirred at 25° C. for 0.5 hr. The reaction mixture was diluted with H₂O 4 mL and extracted with DCM 15 mL (5 mL*3). The combined organic layers were washed with NaCl (aq) 2 mL, dried over Na₂SO₄, filtered, and concentrated under reduced pressure to give I-249-2 (60 mg, crude) as a white solid. LCMS: R^{t}=0.331 min, (M+H)=834.6.

### Step 2: Synthesis of I-249-4

To a solution of I-249-2 (60 mg, 71.90 μmol, 1 eq), I-249-3 (19.65 mg, 71.90 μmol, 1 eq) in DMF (1 mL) was added EDCI (41.35 mg, 215.70 μmol, 3 eq) and HOAt (9.79 mg, 71.90 μmol, 1 eq), NMM (36.36 mg, 359.50 μmol, 39.52 μL, 5 eq). The mixture was stirred at 25° C. for 0.5 hr. The reaction mixture was diluted with H₂O 2 mL and extracted with DCM 8 mL (4 mL*2). The combined organic layers were washed with NaCl (aq) 2 mL, dried over Na₂SO₄, filtered, and concentrated under reduced pressure to give a residue. The residue was purified by flash silica gel chromatography (ISCO®; 12 g SepaFlash® Silica Flash Column, Eluent of 0˜100% Ethyl acetate/Petroleum ether gradient @ 20 mL/min) was concentrated under reduced pressure to give I-249-4 (70 mg, 64.23 μmol, 89.33% yield) as a white solid. LCMS: R^{t}=0.379 min, [M+H]⁺=1089.7. SFC: R^{t}=3.707 min, 4.755 min.

### Step 3: Synthesis of I-249

To a solution of I-249-4 (60 mg, 55.06 μmol, 1 eq) in DCM (0.6 mL) was added TFA (184.20 mg, 1.62 mmol, 120.00 μL, 29.34 eq). The mixture was stirred at 25° C. for 0.5 hr. The reaction mixture was concentrated under reduced pressure to give a residue. The residue was purified by prep-HPLC (column:

CD02-Waters Xbidge BEH C18 150*25*10 um; mobile phase: [water (NH3H₂O)-ACN]; gradient: 48%-78% B over 11 min) the eluent was concentrated to remove ACN and lyophilized to get I-249 (26.23 mg, 25.45 μmol, 46.23% yield, 96.027% purity) as a white solid. LCMS: R^{t}=0.346 min, [M+H]⁺=989.6. SFC: R^{t}=5.620 min, 6.956 min. ¹H NMR (400 MHZ, METHANOL-d4) δ=8.12 (d, J=1.6 Hz, 1H), 7.81-7.66 (m, 2H), 7.55-7.39 (m, 2H), 7.38-7.25 (m, 4H), 7.13-7.11 (m, 1H), 6.64-6.59 (m, 1H), 4.95-4.89 (m, 2H), 4.59-4.44 (m, 3H), 4.42-4.34 (m, 1H), 4.09-3.98 (m, 1H), 3.98-3.79 (m, 1H), 3.74-3.71 (m, 1H), 3.68-3.56 (m, 2H), 3.53-3.42 (m, 2H), 3.29-3.11 (m, 2H), 2.91-2.79 (m, 1H), 2.79-2.65 (m, 3H), 2.40-2.30 (m, 2H), 2.29-2.13 (m, 4H), 2.10-1.93 (m, 5H), 1.93-1.75 (m, 9H), 1.69 (d, J=14.4 Hz, 3H), 1.65-1.56 (m, 4H), 1.55-1.39 (m, 2H), 1.36-1.19 (m, 3H), 1.17-1.05 (m, 2H), 0.97-0.88 (m, 2H), 0.83 (s, 2H).

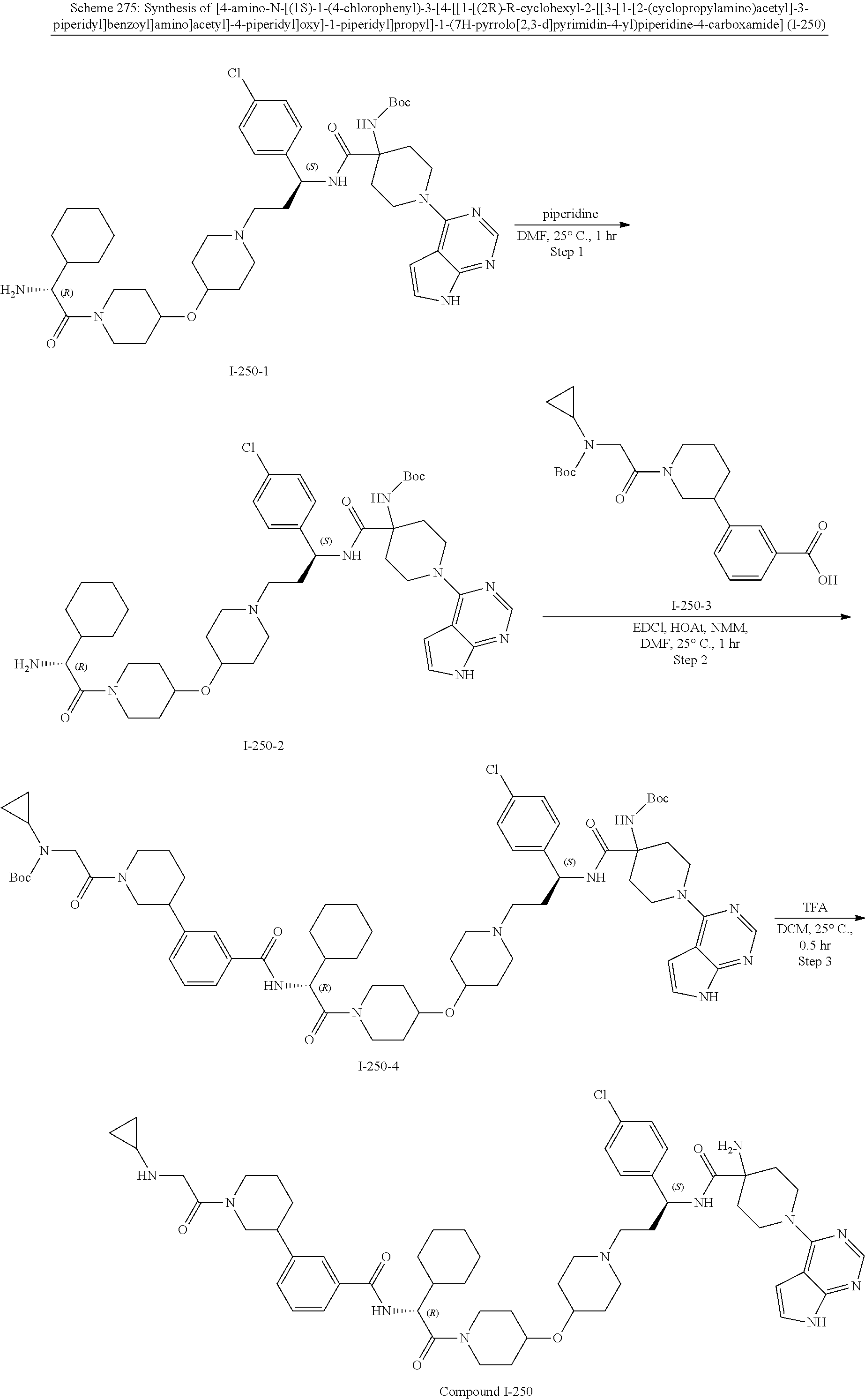

### Step 1: Synthesis of I-250-2.

To a solution of I-250-1 (90 mg, 85.17 μmol, 1 eq) in DMF (1 mL) was added piperidine (14.50 mg, 170.34 μmol, 16.82 μL, 2 eq). The mixture was stirred at 25° C. for 0.5 hr. The reaction mixture was diluted with H₂O 1 mL and extracted with DCM 3 mL (1 mL*3). The combined organic layers were dried over Na₂SO₄, filtered, and concentrated under reduced pressure to give I-250-2 (90 mg, crude) as a yellow oil. LCMS (Method D): R^{t}=0.296 min, [M+H]⁺=834.4.

### Step 2: Synthesis of I-250-4.

To a solution of I-250-2 (90 mg, 107.85 μmol, 1 eq) and I-250-3 (52.09 mg, 129.42 μmol, 1.2 eq) in DMF (1 mL) was added EDCI (62.03 mg, 323.55 μmol, 3 eq), HOAt (14.68 mg, 107.85 μmol, 1 eq) and NMM (54.54 mg, 539.25 μmol, 59.29 μL, 5 eq). The mixture was stirred at 25° C. for 1 hr. The mixture was quenched by addition H₂O 1.0 mL at 25° C., and extracted with DCM 3 mL (1 mL*3), the combined organic phase was dried over Na₂SO₄, filtered, and concentrated under reduced pressure to give a residue. The residue was purified by flash silica gel chromatography (ISCO®; 12 g SepaFlash® Silica Flash Column, Eluent of 0˜100% Dichlormethane: Methanol ether gradient @ 12 mL/min, DCM/MeOH=10/1, Rf=0.3) and the eluent was concentrated to give I-250-4 (100 mg, 81.85 μmol, 75.89% yield, 99.773% purity) as a white solid. LCMS (Method D): R^{t}=0.409 min, [M+H]⁺=1218.9. R^{t}=1.730 min, 2.061 min.

### Step 3: Synthesis of I-250

To a solution of I-250-4 (90 mg, 73.83 μmol, 1 eq) in DCM (1 mL) was added TFA (307.00 mg, 2.69 mmol, 0.2 mL). The mixture was stirred at 25° C. for 0.5 hr. The mixture was dried with N₂ to give a crude product. The crude product was purified by prep-HPLC (column: CD01-Phenomenex luna C18 150*25*10 um; mobile phase: [water (TFA)-ACN]; gradient: 13%-43% B over 10 min), the eluent was concentrated to remove ACN and lyophilized to give I-250 (50.85 mg, 44.89 μmol, 60.80% yield, 100% purity, TFA) as a white solid. LCMS (Method D): R^{t}=0.309 min, [M+H]⁺=1118.4. ¹H NMR (400 MHZ, METHANOL-d4) δ=8.35 (d, J=2.0 Hz, 1H), 8.33-8.24 (m, 1H), 7.80-7.69 (m, 2H), 7.56-7.41 (m, 2H), 7.40-7.33 (m, 5H), 6.89 (t, J=3.2 Hz, 1H), 5.03-4.97 (m, 1H), 4.97-4.91 (m, 1H), 4.65-4.60 (m, 2H), 4.58-4.42 (m, 1H), 4.33-4.26 (m, 1H), 4.25-4.13 (m, 1H), 4.06-3.97 (m, 1H), 3.92 (s, 1H), 3.87-3.72 (m, 5H), 3.61-3.47 (m, 2H), 3.22 (d, J=10.8 Hz, 2H), 3.19-3.12 (m, 1H), 3.11-2.98 (m, 2H), 2.94-2.83 (m, 1H), 2.83-2.74 (m, 2H), 2.69-2.54 (m, 2H), 2.35-2.32 (m, 1H), 2.29-2.12 (m, 3H), 2.11-1.97 (m, 4H), 1.96-1.82 (m, 7H), 1.77 (d, J=1.6 Hz, 2H), 1.73-1.58 (m, 5H), 1.57-1.48 (m, 1H), 1.37-1.04 (m, 6H), 0.95-0.88 (m, 4H). ¹⁹F NMR (376 MHz, METHANOL-d4) δ=−76.96 (s, 1F), −113.94 (br dd, J=5.7, 22.6 Hz, 2F), −120.75 (br d, J=28.3 Hz, 1F), —F.

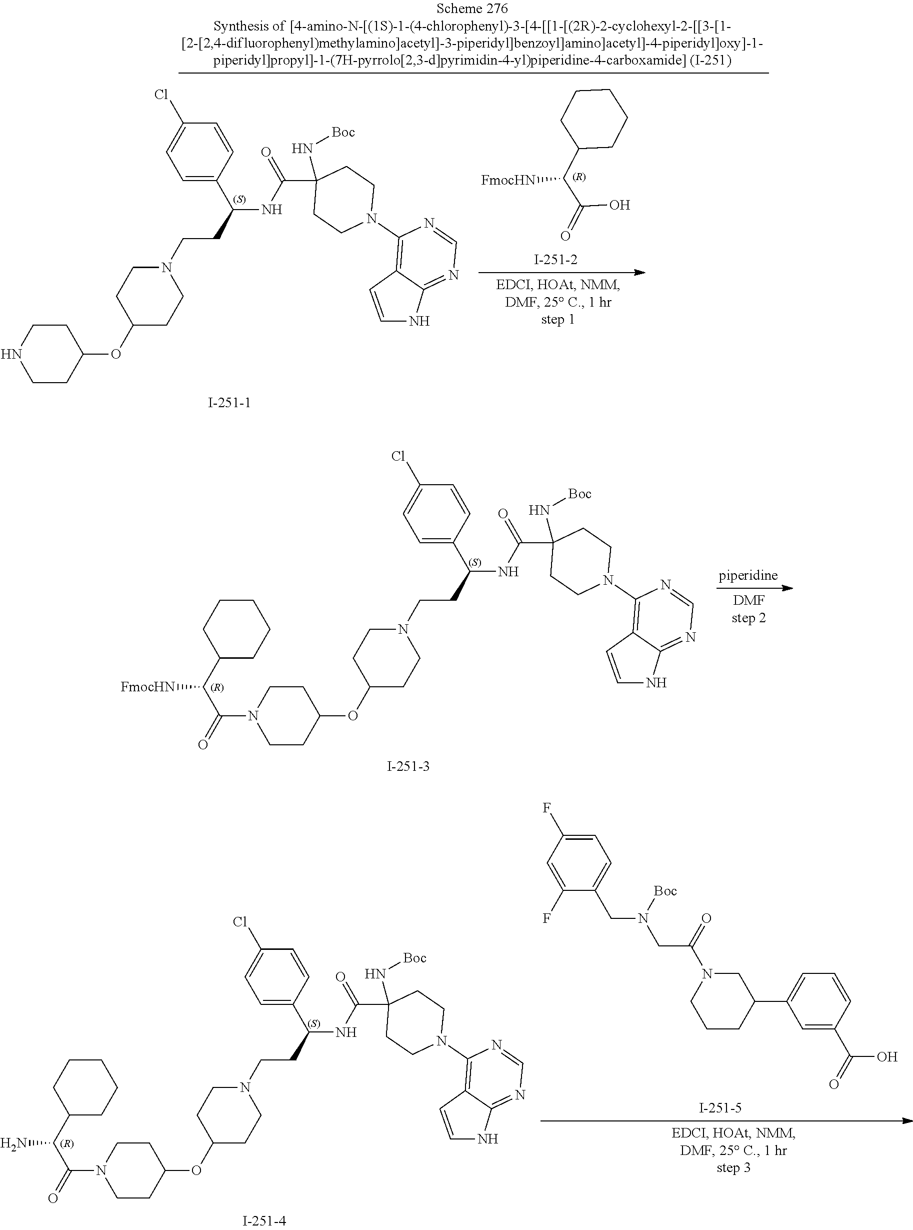

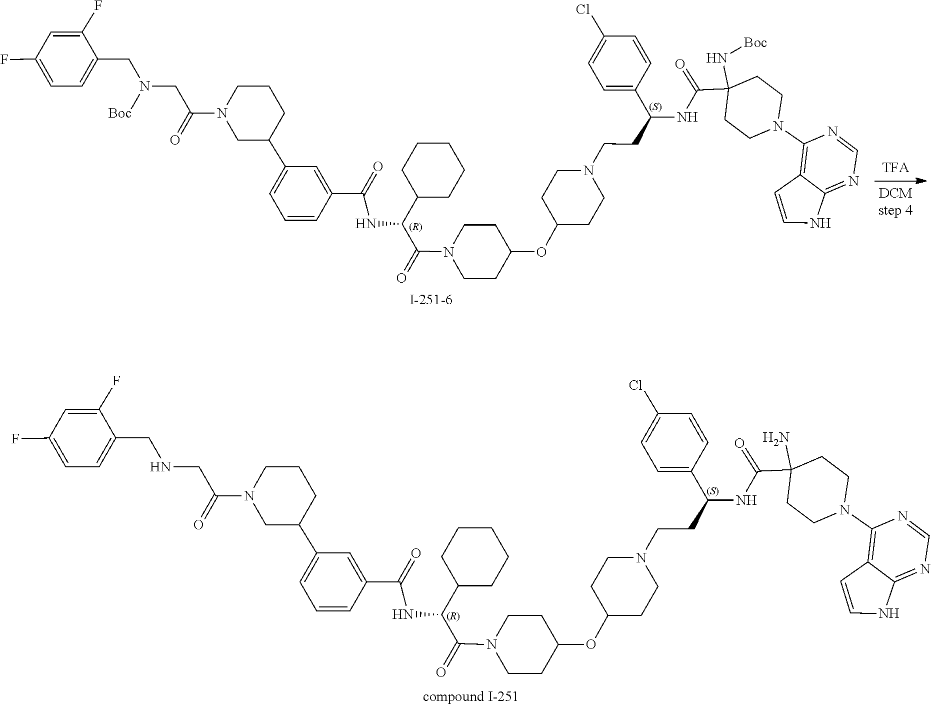

F.

### Step 1: Synthesis of I-251-3

To a solution of I-251-1 (1.8 g, 2.20 mmol, 1 eq) in DMF (18 mL) was added EDCI (1.27 g, 6.60 mmol, 3 eq), NMM (1.11 g, 11.00 mmol, 1.21 mL, 5 eq), I-251-2 (834.98 mg, 2.20 mmol, 834.98 UL, 1 eq) and HOAt (299.51 mg, 2.20 mmol, 307.83 μL, 1 eq). The mixture was stirred at 25° C. for 15 min. The crude product was purified by reversed phase column (0.1% FA condition) and the eluent was lyophilized to give I-251-3 (1.6 g, 1.48 mmol, 67.43% yield, 98% purity) as a white solid. SFC: R^{t}=1.769 min. LCMS (Method D): R^{t}=0.409 min, [M+H]⁺=1056.7.

### Step 2: Synthesis of I-251-4

To a solution of I-251-3 (100 mg, 94.63 μmol, 1 eq) in DMF (1 mL) was added piperidine (24.17 mg, 283.90 μmol, 28.04 μL, 3 eq). The mixture was stirred at 25° C. for 0.5 h. The crude product was purified by reversed phase column (neutral condition) and the eluent was lyophilized to give I-251-4 (80 mg, 81.49 μmol, 86.11% yield, 85% purity) as a yellow oil. LCMS (Method D): R^{t}=0.306 min, [M+H]⁺=834.4.

### Step 3: Synthesis of I-251-6

To a solution of I-251-4 (70 mg, 83.88 μmol, 1 eq) in DMF (0.7 mL) was added EDCI (48.24 mg, 251.65 μmol, 3 eq), NMM (42.42 mg, 419.42 μmol, 46.11 μL, 5 eq), I-251-5 (40.98 mg, 83.88 μmol, 1 eq) and HOAt (11.42 mg, 83.88 μmol, 1 eq). The mixture was stirred at 25° C. for 15 min. (SiO₂, by UV 254 nm, DCM:MeOH=10:1, Rf=0.6). The crude product was purified by column chromatography (SiO₂, EA/MeOH=1/0 to 0/1) and the eluent was concentrated under reduced pressure to give I-251-6 (60 mg, 45.52 μmol, 54.26% yield, 99% purity) as a white solid. SFC: R^{t}=2.129 min, 2.194 min. LCMS (Method D): R^{t}=0.430 min, [M/2+H]⁺=1304.8.

### Step 4: Synthesis of I-251

To a solution of I-251-6 (50 mg, 38.31 μmol, 1 eq) in DCM (0.6 mL) was added TFA (307.00 mg, 2.69 mmol, 0.2 mL, 70.27 eq). The mixture was stirred at 25° C. for 0.5 h. Concentrated under reduced pressure to give a residue. The crude product was purified by reversed phase column (0.1% TFA condition) and the eluent was lyophilized to give crude product. The crude product was second purified by prep-HPLC (column: CD04-Welch Ultimate C18 150*25*7 um; mobile phase: [water (TFA)-ACN]; gradient: 12%-42% B over 11 min) and the eluent was lyophilized to give I-251 (18.23 mg, 14.96 μmol, 39.04% yield, 100% purity, TFA) as a white solid. SFC: R^{t}=5.769 min, 6.272 min. LCMS (Method D): R^{t}=0.322 min, [M+H]⁺=1104.6. ¹H NMR (400 MHZ, METHANOL-d4) δ=8.33 (d, J=2.0 Hz, 1H), 7.82-7.69 (m, 2H), 7.66-7.57 (m, 1H), 7.53-7.42 (m, 2H), 7.40-7.31 (m, 5H), 7.20-7.04 (m, 2H), 6.86 (t, J=3.6 Hz, 1H), 5.05-4.93 (m, 2H), 4.64-4.42 (m, 3H), 4.32 (d, J=13.2 Hz, 2H), 4.27-4.10 (m, 2H), 4.08-3.98 (m, 1H), 3.92 (s, 1H), 3.87-3.44 (m, 8H), 3.36 (br d, J=2.0 Hz, 1H), 3.27-3.17 (m, 3H), 3.14-3.00 (m, 2H), 2.96-2.71 (m, 2H), 2.68-2.52 (m, 2H), 2.43-2.11 (m, 4H), 2.10-1.97 (m, 4H), 1.93 (d, J=1.2 Hz, 6H), 1.77 (s, 3H), 1.68 (s, 4H), 1.58-1.49 (m, 1H), 1.34-1.20 (m, 3H), 1.17-1.06 (m, 2H).

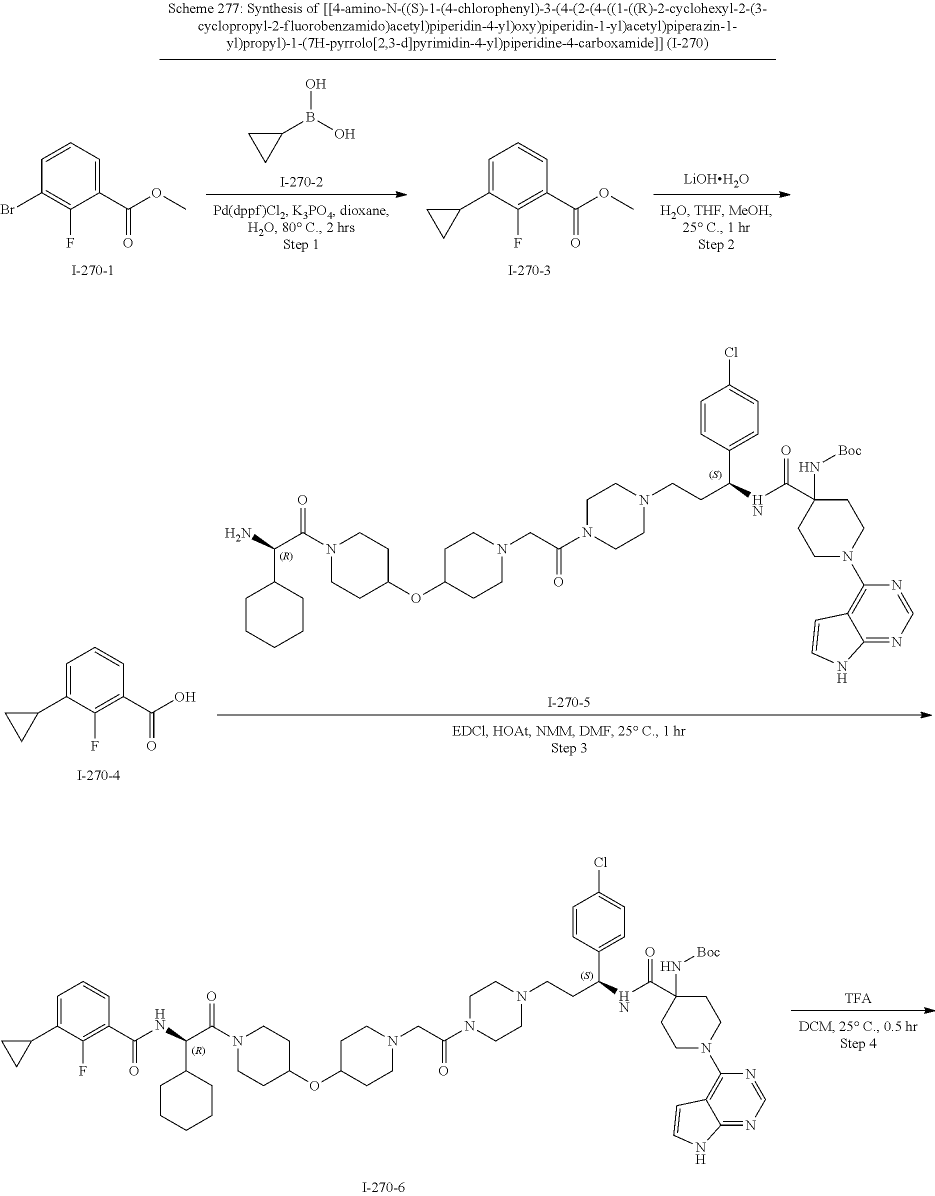

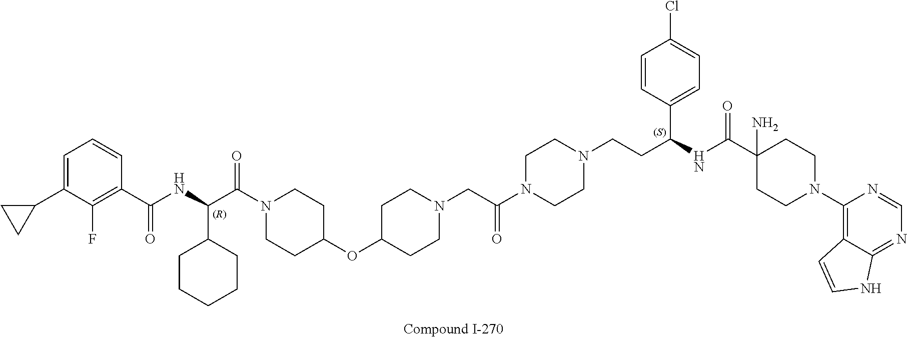

### Step 1: Synthesis of I-270-3

A mixture of I-270-1 (2 g, 8.58 mmol, 1 eq), I-270-2 (1.11 g. 12.87 mmol, 1.5 eq), Pd(dppf)Cl₂ (559.36 mg, 858.24 μmol, 0.1 eq), K₃PO₄ (5.47 g, 25.75 mmol, 3 eq) and in dioxane (20 mL) and H₂O (4 mL) was degassed and purged with N₂ for 3 times. Then the mixture was stirred at 80° C. for 2 hr under N₂ atmosphere. The reaction mixture was quenched by addition H₂O 20 mL, and extracted with DCM 15 mL (5 mL*3). The combined organic layers were dried over Na₂SO₄, filtered, and concentrated under reduced pressure to give a crude product. The residue was purified by flash silica gel chromatography (ISCO®; 40 g SepaFlash® Silica Flash Column, Eluent of 0˜100% Dichloromethane: Methanol gradient (@ 20 mL/min, PE/EA=3/1, R_{f}=0.5) and the eluent was concentrated to give product. I-270-3 (1.48 g, 7.29 mmol, 84.93% yield, 95.645% purity) was obtained as a red oil. LCMS (Method D): R^{t}=0.407 min, [M+H]⁺=195.1. ¹H NMR (400 MHZ, METHANOL-d4) δ=7.69-7.59 (m, 1H), 7.20-7.07 (m, 2H), 3.89 (s, 3H), 2.16-2.06 (m, 1H), 1.04-0.97 (m, 2H), 0.76-0.68 (m, 2H).

### Step 2: Synthesis of I-270-4

To a solution of I-270-3 (1.38 g, 7.11 mmol, 1 eq) in H₂O (5 mL), THF (5 mL) and MeOH (5 mL) was added LiOH·H₂O (894.58 mg, 21.32 mmol, 3 eq). The mixture was stirred at 25° C. for 1 hr. The mixture was adjusted the pH to acid with 1M HCl and quenched by addition H₂O 15 mL at 25° C., and extracted with DCM 15 mL (5 mL*3). The combined organic layers were dried over Na₂SO₄, filtered, and concentrated under reduced pressure to give a crude product. The crude product was used to next step. I-270-4 (0.68 g, 3.54 mmol, 49.79% yield, 93.740% purity) was obtained as a yellow solid. LCMS (Method D): R^{t}=0.337 min, [M+H]⁺=181.1.

### Step 3: Synthesis of I-270-6

To a solution of I-270-4 (0.45 g, 468.44 μmol, 1 eq), I-270-5 (101.28 mg, 562.12 μmol, 1.2 eq) in DMF (5 mL) was added EDCI (269.40 mg, 1.41 mmol, 3 eq) and HOAt (63.76 mg, 468.44 μmol, 65.53 μL, 1 eq), NMM (236.90 mg, 2.34 mmol, 257.51 μL, 5 eq). The mixture was stirred at 25° C. for 1 hr. The reaction mixture was diluted with H₂O 15 mL and extracted with DCM 50 mL (25 mL*2). The combined organic layers were washed with NaCl (aq) 20 mL, dried over Na₂SO₄, filtered, and concentrated under reduced pressure to give a residue. The residue was purified by prep-HPLC (column: CD07-Daisogel SP-100-8-ODS-PK 150*25*10 um; mobile phase: [water (NH₄HCO₃)-ACN]; gradient: 55%-75% B over 10 min) the eluent was concentrated to remove ACN and lyophilized to get I-270-6 (0.35 g, 311.72 μmol, 66.54% yield) as a white solid. SFC: R^{t}=1.718 min.

### Step 4: Synthesis of I-270

To a solution of I-270-6 (0.35 g, 311.72 μmol, 1 eq) in DCM (4 mL) was added TFA (1.23 g, 10.77 mmol, 0.8 mL, 34.55 eq). The mixture was stirred at 25° C. for 0.5 hr. The reaction mixture was concentrated under reduced pressure to give a residue. The residue was purified by reverse-phase column (0.1% TFA) the eluent was concentrated to remove ACN and lyophilized to get I-270 (98.73% purity, TFA), which was obtained as a white solid. Special LCMS: R^{t}=12.772 min, [M+H]⁺=1022.5. SFC: R^{t}=1.986 min. ¹H NMR (400 MHz, METHANOL-d4) δ=8.37 (s, 1H), 7.48-7.44 (m, 1H), 7.40-7.29 (m, 5H), 7.18-7.10 (m, 2H), 6.91 (d, J=3.6 Hz, 1H), 5.05-5.01 (m, 1H), 4.96 (d, J=7.8 Hz, 1H), 4.70-4.58 (m, 2H), 4.30 (d, J=7.6 Hz, 2H), 4.07-4.00 (m, 1H), 3.98-3.61 (m, 10H), 3.56-3.33 (m, 7H), 3.26-3.08 (m, 3H), 2.73-2.53 (m, 2H), 2.41-2.25 (m, 2H), 2.22-2.09 (m, 3H), 2.09-1.93 (m, 5H), 1.91-1.74 (m, 6H), 1.70 (d, J=11.2 Hz, 3H), 1.61-1.49 (m, 1H), 1.36-1.21 (m, 3H), 1.20-1.08 (m, 2H), 1.06-0.97 (m, 2H), 0.79-0.70 (m, 2H). ¹⁹F NMR (377 MHz, METHANOL-d4) δ=−77.12 (s, 10F), −123.32 (d, J=14.1 Hz, 1F).

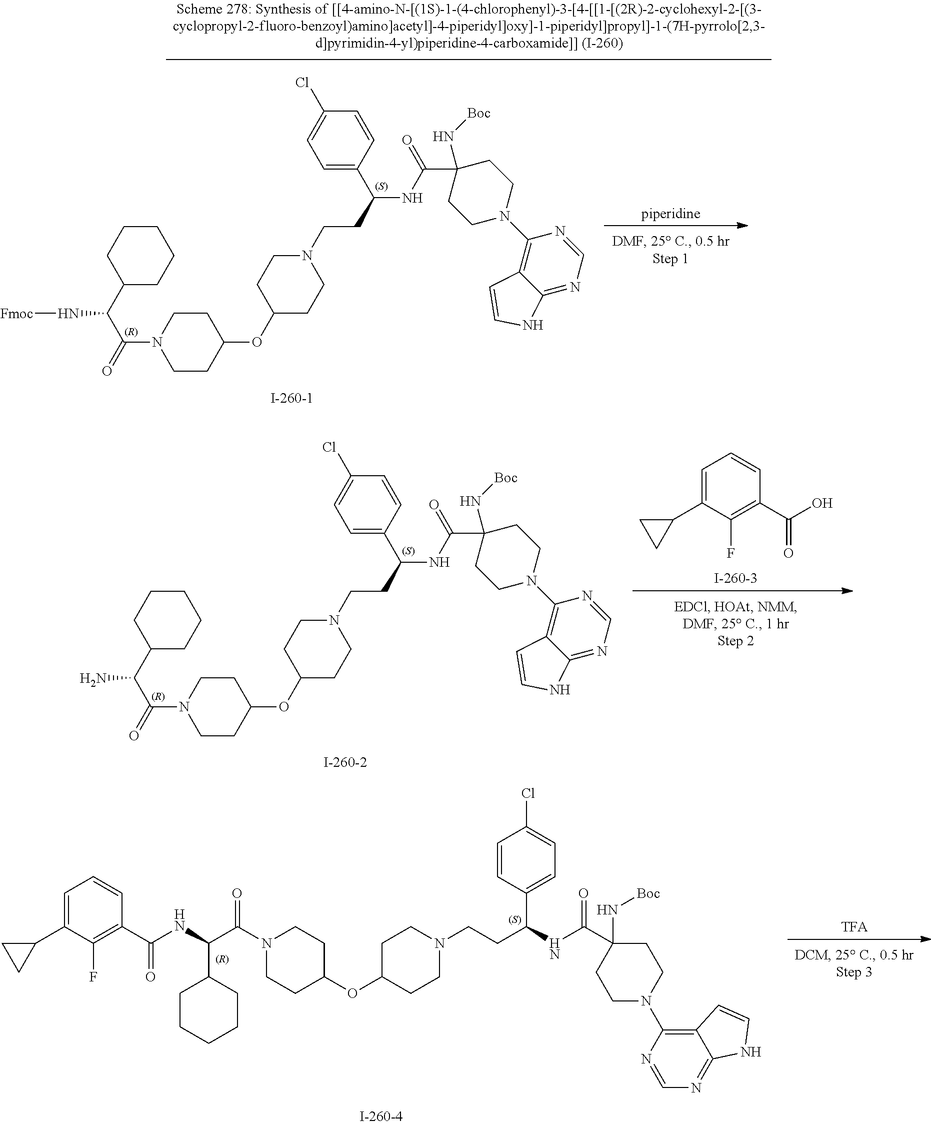

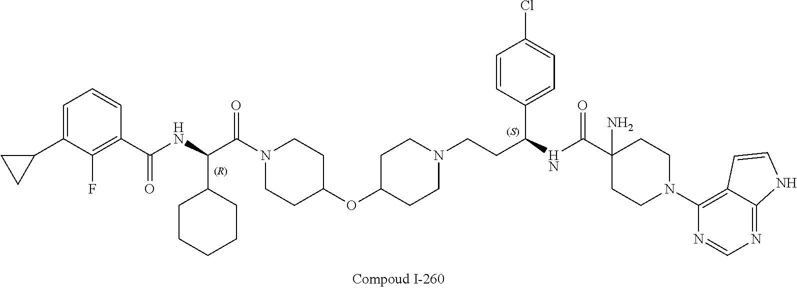

### Step 1: Synthesis of I-280-2.

To a solution of I-280-1 (360 mg, 340.68 μmol, 1 eq) in DMF (3.6 mL) was added piperidine (58.02 mg, 681.35 μmol, 67.29 μL, 2 eq). The mixture was stirred at 25° C. for 0.5 h. The mixture was quenched by addition H₂O 4 mL at 25° C., and extracted with DCM 12 mL (4 mL*3). The combined organic phase was dried over dried over Na₂SO₄, filtered, and concentrated under reduced pressure to give a crude product. The crude product was used into the next step without further purification. I-280-2 (360 mg, crude) was obtained as a yellow oil. LCMS (Method D): R^{t}=0.298 min, [M+H]⁺=834.3.

### Step 2: Synthesis of I-280-4.

To a solution of I-280-2 (360 mg, 431.40 μmol, 1 eq) and I-280-3 (85.50 mg, 474.54 μmol, 1.1 eq) in DMF (4 mL) was added EDCI (248.10 mg, 1.29 mmol, 3 eq), NMM (218.18 mg, 2.16 mmol, 237.15 μL, 5 eq) and HOAt (58.72 mg, 431.40 μmol, 60.35 μL, 1 eq). The mixture was stirred at 25° C. for 1 h. The mixture was quenched by addition H₂O 4 mL at 25° C., and extracted with DCM 12 mL (4 mL*3). The combined organic phase was dried over dried over Na₂SO₄, filtered, and concentrated under reduced pressure to give a crude product. The crude product was purified by prep-HPLC (column: CD06-Waters Xbidge C18 150*40*10 um; mobile phase: [water (TFA)-ACN]; gradient: 37%-67% B over 14 min), the eluent was concentrated to remove ACN and lyophilized to give the product. I-280-4 (340 mg, 339.47 μmol, 78.69% yield, 99.509% purity) was obtained as a white solid. LCMS (Method D): R^{t}=0.395 min, [M+H]⁺=996.5. SFC: R^{t}=0.727 min.

### Step 3: Synthesis of I-260

To a solution of I-280-4 (330 mg, 331.11 μmol, 1 eq) in DCM (3 mL) was added TFA (921.00 mg, 8.08 mmol, 0.6 mL). The mixture was stirred at 25° C. for 0.5 h. The mixture was dried with N₂ to give a crude product. The crude product was purified by reverse-phase column (0.1% TFA condition), the eluent was concentrated to remove ACN and lyophilized to give the product. I-260 (200.3 mg, 192.48 μmol, 52.29% yield, 97.11% purity, TFA) was obtained as a white solid. LCMS (Method D): R^{t}=0.356 min, [M+H]⁺=896.6. Special LCMS: R^{t}=14.085 min, [M+H]⁺=896.3. SFC: R^{t}=1.672 min. ¹H NMR (400 MHz, METHANOL-d4) δ=8.35 (d, J=2.4 Hz, 1H), 7.49-7.42 (m, 1H), 7.37 (d, J=3.6 Hz, 4H), 7.34 (t, J=3.6 Hz, 1H), 7.18-7.09 (m, 2H), 6.87 (t, J=3.6 Hz, 1H), 5.04-4.97 (m, 1H), 4.95 (d, J=8.0 Hz, 1H), 4.73-4.61 (m, 2H), 4.06-3.98 (m, 1H), 3.95-3.84 (m, 1H), 3.84-3.69 (m, 4H), 3.68-3.54 (m, 1H), 3.53-3.45 (m, 1H), 3.42-3.36 (m, 1H), 3.27-3.12 (m, 3H), 3.06 (s, 2H), 2.69-2.51 (m, 2H), 2.40-2.30 (m, 1H), 2.27-2.20 (m, 1H), 2.17-2.09 (m, 2H), 2.08-1.99 (m, 2H), 1.99-1.91 (m, 2H), 1.91-1.73 (m, 6H), 1.73-1.64 (m, 3H), 1.59-1.48 (m, 1H), 1.36-1.07 (m, 6H), 1.06-0.99 (m, 2H), 0.79-0.71 (m, 2H). ¹⁹F NMR (376 MHz, METHANOL-d4) δ=−77.12 (s, 9F), −123.32 (br d, J=17.0 Hz, 1F).

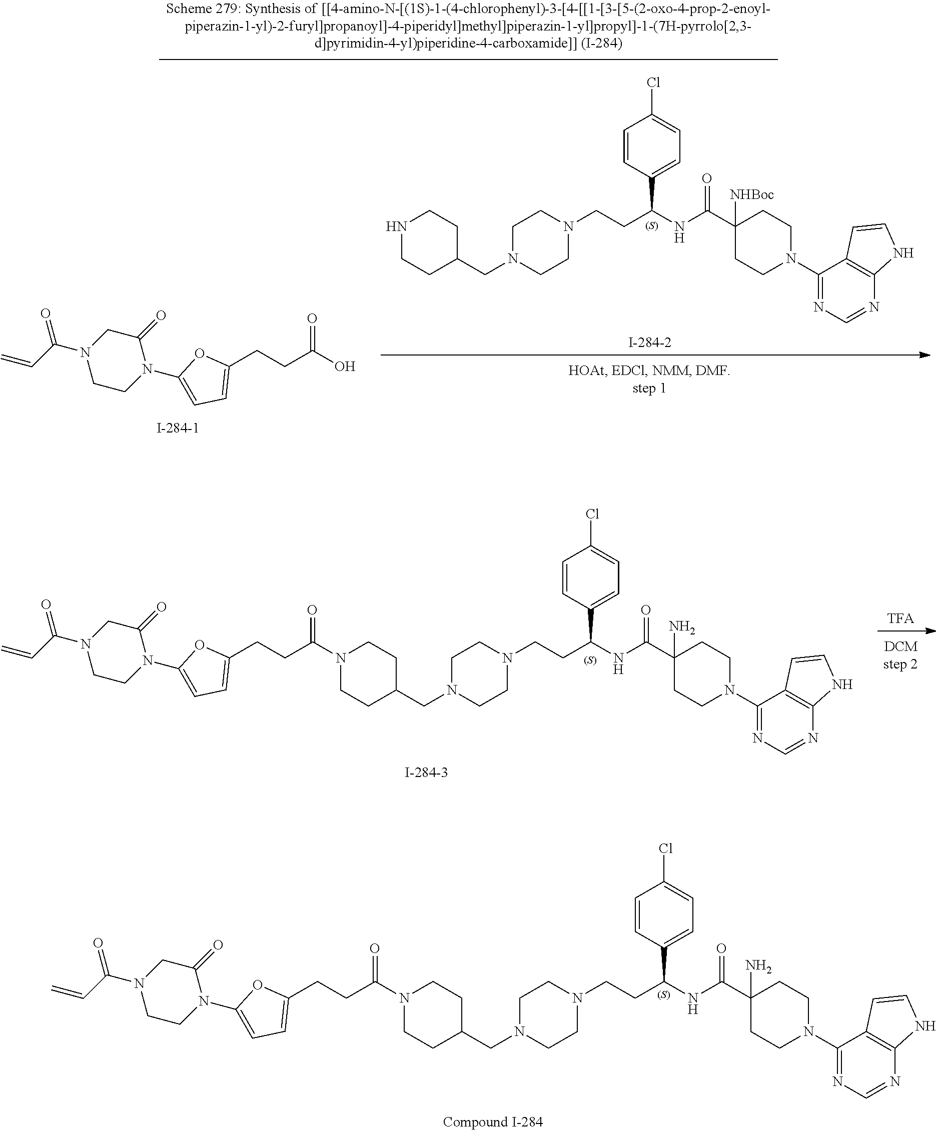

### Step 1: Synthesis of I-284-3

To a solution of I-284-1 (63.15 mg, 216.04 μmol, 1 eq) and I-284-2 (150 mg, 216.04 μmol, 1 eq) in DMF (2 mL) was added HOAt (29.41 mg, 216.04 μmol, 30.22 μL, 1 eq), EDCI (124.25 mg, 648.13 μmol, 3 eq) and NMM (109.26 mg, 1.08 mmol, 118.76 μL, 5 eq), then the mixture was stirred at 25° C. for 1 hour. The reaction mixture was diluted with water 20 mL and extracted with DCM 15 mL (5 mL*3). The combined organic layers were concentrated under reduced pressure to give a residue. The crude product was purified by reverse-phase column (0.1% FA condition). The eluent was concentrated under reduced pressure to remove ACN and then lyophilized to give the product. I-284-2 (100 mg, 85.69 μmol, 39.66% yield, 83% purity) was obtained as a white solid. LCMS (Method D): R^{t}=0.316 min, [M+H]⁺=968.4.

### Step 2: Synthesis of I-284

To a solution of I-284-3 (60 mg, 61.95 μmol, 1 eq) in DCM (1 mL) was added TFA (0.2 mL), then the mixture was stirred at 25° C. for 1 hour. The reaction mixture was added NaHCO₃ about 10 mL to adjust to a pH=7˜8, extracted with DCM 30 mL (10 mL*3). The combined organic layers were concentrated under reduced pressure to give a residue. The crude product was purified by reversed phase column (0.1% FA condition), then the eluent was concentrated to remove ACN and lyophilized to get the product. I-284 (23.92 mg, 26.10 μmol, 42.13% yield, 99.77% purity, FA) was obtained as a white solid. LCMS (Method D): R^{t}=0.268 min, [M+H]⁺=868.5. ¹H NMR (400 MHZ, METHANOL-d4) δ=8.54 (s, 1H), 8.13 (s, 1H), 7.32 (d, J=1.6 Hz, 4H), 7.12 (d, J=3.6 Hz, 1H), 6.87-6.66 (m, 1H), 6.62 (d, J=3.6 Hz, 1H), 6.32-6.27 (m, 1H), 6.24 (d, J=3.2 Hz, 1H), 6.11 (d, J=3.2 Hz, 1H), 5.84-5.80 (m, 1H), 4.94-4.91 (m, 1H), 4.55-4.35 (m, 5H), 4.04-3.79 (m, 5H), 3.69-3.58 (m, 2H), 3.17-2.80 (m, 4H), 2.74-2.67 (m, 2H), 2.64-2.55 (m, 2H), 2.46 (s, 5H), 2.34-2.25 (m, 2H), 2.25-2.13 (m, 2H), 2.08 (d, J=6.4 Hz, 2H), 2.05-1.92 (m, 2H), 1.80-1.65 (m, 4H), 1.65-1.55 (m, 2H), 1.04-0.89 (m, 2H).

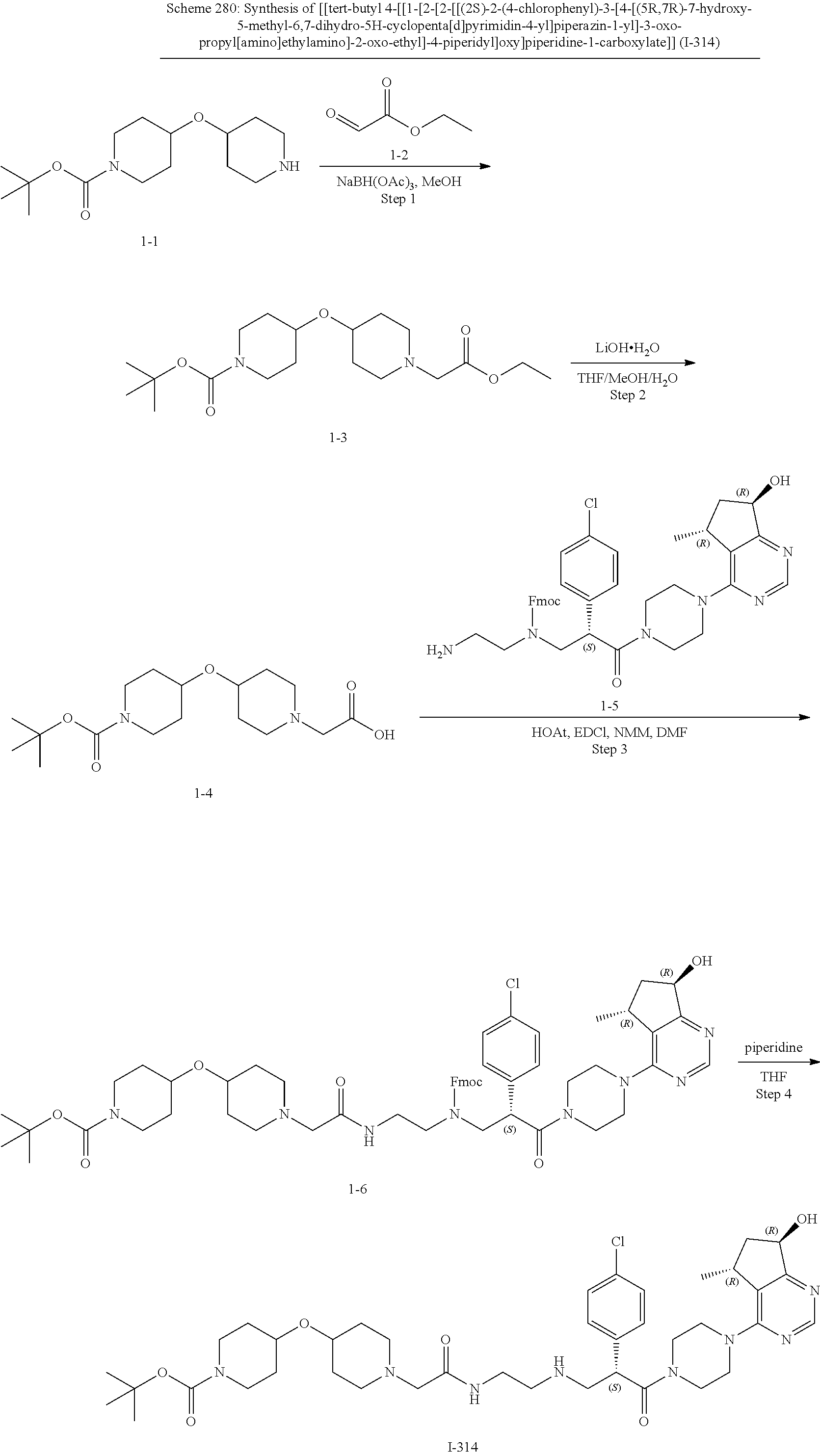

### Step 1: Synthesis of 1-3.

To a mixture of 1-1 (500 mg, 1.76 mmol, 1 eq) and 1-2 (179.48 mg, 1.76 mmol, 1 eq) in MeOH (6 mL) was added AcOH (10.56 mg, 175.81 μmol, 10.06 μL, 0.1 eq), stirred at 20° C. for 0.5 hour, then NaBH(OAc)₃ (1.86 g, 8.79 mmol, 5 eq) was added to the mixture and stirred at 20° C. for 0.5 hour. The reaction mixture was diluted with water 50 mL and extracted with DCM 30 mL (10 mL*3). The combined organic layers were concentrated under reduced pressure to give 1-3 (0.3 g, 809.76 μmol, 46.06% yield) as a yellow oil. LCMS: R^{t}=0.279 min, [M+H]⁺=371.2.

### Step 2: Synthesis of 1-4.

To a mixture of 1-3 (300 mg, 809.76 μmol, 1 eq) in THF (2 mL), MeOH (1 mL) and H₂O (0.5 mL) was added LiOH·H₂O (101.94 mg, 2.43 mmol, 3 eq), then the mixture was stirred at 20° C. for 1 hour. The reaction mixture was concentrated under reduced pressure to remove THF and MeOH, then added 1 N HCl about 2 mL to adjust to a pH=1˜2, extracted with DCM 10 mL (5 mL*2). The combined organic layers were concentrated under reduced pressure to give 1-4 (100 mg, 292.03 μmol, 36.06% yield) as a white solid. LCMS: R^{t}=0.280 min, [M+H]⁺=343.1.

### Step 3: Synthesis of 1-6.

To a mixture of 1-4 (90 mg, 262.83 μmol, 1 eq) and 1-5 (179.04 mg, 262.83 μmol, 1 eq) in DMF (1 mL) was added HOAt (35.77 mg, 262.83 μmol, 36.77 μL, 1 eq), EDCI (151.15 mg, 788.48 μmol, 3 eq) and NMM (132.92 mg, 1.31 mmol, 144.48 μL, 5 eq), then the mixture was stirred at 20° C. for 1 hour. The reaction mixture was diluted with water 30 mL and extracted with DCM 30 mL (10 mL*3). The combined organic layers were concentrated under reduced pressure to give 1-6 (100 mg, 99.44 μmol, 37.83% yield) as a yellow solid. LCMS (Method D): R^{t}=0.391 min, [M+H]⁺=1005.5.

### Step 4: Synthesis of I-314

To a solution of 1-6 (80 mg, 79.55 μmol, 1 eq) in THF (0.8 mL) was added PIPERIDINE (0.1 mL) stirred at 20° C. for 10 min. The reaction mixture was concentrated under reduced pressure to give a residue. The crude product was purified by reverse-phase column (0.1% NH₃·H₂O condition). The eluent was concentrated under reduced pressure to remove MeCN and then lyophilized to give I-314 (15.14 mg, 19.04 μmol, 23.93% yield, 98.50% purity) as a white solid. LCMS (Method D): R^{t}=0.286 min, [M+H]⁺=783.6. SFC: R^{t}=1.827 min. ¹H NMR (400 MHZ, METHANOL-d4) δ=8.41 (s, 1H), 7.41-7.35 (m, 2H), 7.35-7.30 (m, 2H), 4.99-4.94 (m, 1H), 4.21-4.17 (m, 1H), 3.95-3.90 (m, 13.0 Hz, 1H), 3.82-3.42 (m, 12H), 3.34 (d, J=2.0 Hz, 2H), 3.27-3.24 (m, 1H), 3.17-3.07 (m, 2H), 2.97 (s, 2H), 2.82-2.70 (m, 5H), 2.29-2.26 (m, 2H), 2.17-2.02 (m, 2H), 1.90-1.76 (m, 4H), 1.68-1.57 (m, 2H), 1.50-1.46 (m, 1H), 1.45 (s, 9H), 1.43-1.38 (m, 1H), 1.12 (d, J=6.8 Hz, 3H).

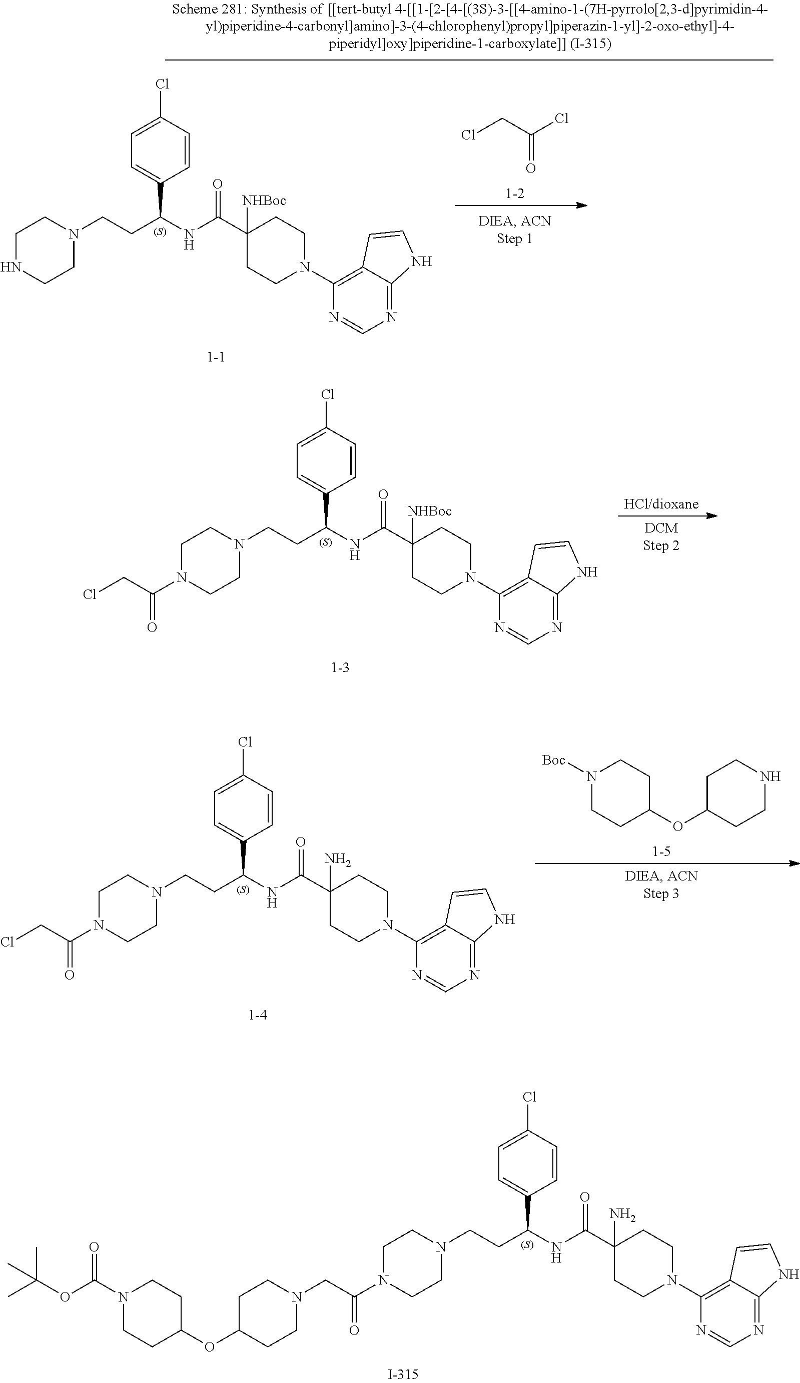

To a solution of 1-1 (100 mg, 167.46 μmol, 1 eq) in ACN (1 mL) was added DIEA (64.93 mg, 502.39 μmol, 87.51 μL, 3 eq) and 1-2 (28.37 mg, 251.19 μmol, 20.01 μL, 1.5 eq) at 0° C. The mixture was stirred at 0° C. for 1 hr. The reaction mixture diluted with H₂O (1.5 mL) and extracted with EA (2 mL*2), the combined organic layers were dried over Na₂SO₄, filtered, and concentrated under reduced pressure to give a residue. The filter liquor was purified by reverse-phase column (0.1% FA condition). The eluent was concentrated under reduced pressure to remove ACN and then lyophilized to give 1-3 (85 mg, 118.11 μmol, 70.53% yield, FA salt) as a yellow solid. LCMS (Method D): R^{t}=0.301 min, [M+H]⁺=673.2. ¹H NMR (400 MHZ, METHANOL-d4) δ=8.19 (s, 1H), 8.13 (s, 1H), 7.37-7.27 (m, 4H), 7.13 (d, J=3.6 Hz, 1H), 6.63 (d, J=3.6 Hz, 1H), 5.07-4.99 (m, 1H), 4.46-4.31 (m, 2H), 4.25 (s, 2H), 3.73-3.55 (m, 6H), 2.65-2.41 (m, 6H), 2.21-1.97 (m, 6H), 1.54-1.38 (m, 9H).

### Step 2: Synthesis of 1-4

To a solution of 1-3 (75 mg, 104.22 μmol, 1 eq, FA salt) in CH₂Cl₂ (0.6 mL) and HCl/dioxane (2 M, 0.6 mL, 11.51 eq) was stirred at 25° C. for 1 hr. The mixture was concentrated under reduced pressure to give 1-4 (65 mg, crude, HCl salt) as a yellow solid. LCMS: R^{t}=0.288 min, [M+H]⁺=573.0.

### Step 3: Synthesis of I-315

To a solution of 1-4 (55 mg, 90.17 μmol, 1 eq, HCl salt) in ACN (1 mL) was added DIEA (34.96 mg, 270.50 μmol, 47.12 μL, 3 eq) and 1-5 (25.64 mg, 90.17 μmol, 1 eq). The mixture was stirred at 40° C. for 1 hr. The mixture was filtered. The filtrate was purified by reverse-phase column (0.1% FA condition). The eluent was concentrated under reduced pressure to remove ACN and then lyophilized to give the crude. The crude was purified by prep-HPLC (column: CD01-Phenomenex luna C18 150*25*10 um; mobile phase: [water (FA)-ACN]; gradient: 25%-55% B over 10 min). The eluent was concentrated under reduced pressure to remove ACN and then lyophilized to give I-315 (11.47 mg, 13.22 μmol, 14.66% yield, 100% purity, FA salt) as a white solid. LCMS (Method D): R^{t}=0.272 min, [M+H]⁺=821.4. ¹H NMR (400 MHZ, METHANOL-d4) δ=8.43 (s, 1H), 8.16 (s, 1H), 7.38-7.25 (m, 4H), 7.16 (d, J=3.6 Hz, 1H), 6.65 (d, J=3.6 Hz, 1H), 5.05-4.96 (m, 1H), 4.64-4.51 (m, 2H), 3.97 (s, 2H), 3.79 (s, 1H), 3.76-3.65 (m, 4H), 3.65-3.59 (m, 3H), 3.44 (s, 2H), 3.29-3.25 (m, 1H), 3.19-3.06 (m, 4H), 2.53-2.41 (m, 4H), 2.40-2.21 (m, 4H), 2.08-1.97 (m, 4H), 1.91-1.74 (m, 6H), 1.52-1.41 (m, 13H). SFC: R^{t}=1.548 min.

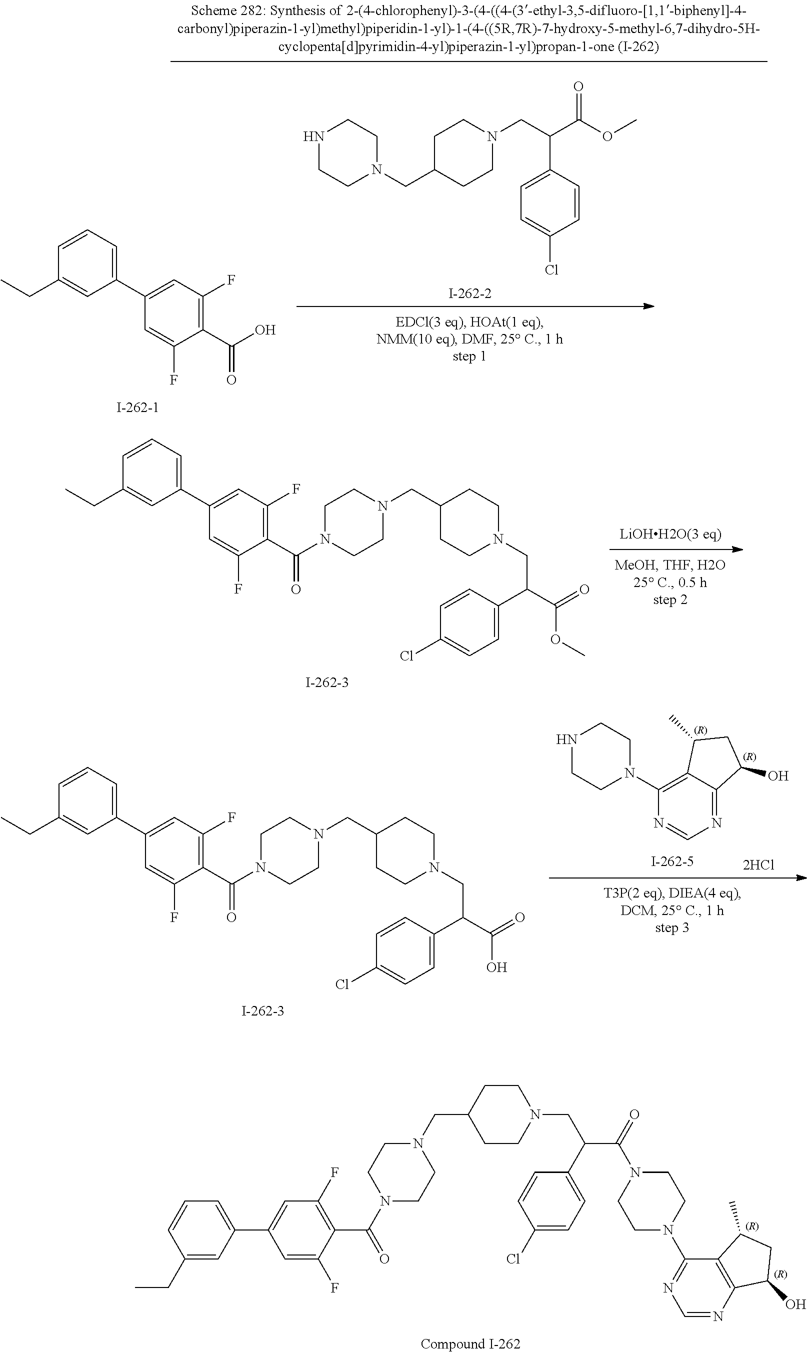

### Step 1: Synthesis of I-262-3

To a solution of I-262-1 (300 mg, 1.14 mmol, 1 eq) I-262-2 (476.32 mg, 1.14 mmol, 1 eq, HCl) in DMF (3 mL) was added EDCI (657.88 mg, 3.43 mmol, 3 eq), HOAt (155.70 mg, 1.14 mmol, 160.02 μL, 1 eq) and NMM (1.16 g, 11.44 mmol, 1.26 mL, 10 eq). The mixture was stirred at 25° C. for 1 h. The reaction mixture was quenched by addition H₂O 3 mL, and then diluted with EA 3 mL and extracted with EA 15 mL (5 mL*3). The combined organic layers were washed with brine 15 mL (5 mL*3), dried over anhydrous Na: SO₄, filtered, and concentrated under reduced pressure to give a residue. The residue was purified by flash silica gel chromatography (ISCO®; 20 g SepaFlash® Silica Flash Column, Eluent of 0˜100% Ethyl acetate/Petroleum ether gradient @ 45 mL/min). concentrated under reduced pressure to give I-262-3 (280 mg, 444.12 μmol, 38.82% yield, 99% purity) as a white solid. LCMS: R^{t}=0.474 min, [M+H]⁺=624.3. SFC: R^{t}=2.183 min, 2.324 min.

### Step 2: Synthesis of I-262-4

To a solution of I-262-3 (280 mg, 448.60 μmol, 1 eq) in MeOH (1.5 mL), THF (1.5 mL) and H₂O (0.5 mL) was added LiOH·H₂O (56.48 mg, 1.35 mmol, 3 eq). The mixture was stirred at 80° C. for 1 h. The reaction mixture was adjusted to a pH=6 with aq. HCl (1 M), and then diluted with EA 3 mL and extracted with EA 9 mL (3 mL*3). The combined organic layers were dried over anhydrous Na₂SO₄, concentrated under reduced pressure to give a residue which was used in next step without further purification. I-262-4 (250 mg, 385.16 μmol, 85.86% yield, 94% purity) was obtained as a white solid. LCMS: R^{t}=0.465 min,

[M+H]⁺=610.3.

### Step 3: Synthesis of I-262

To a solution of I-262-4 (100 mg, 163.90 μmol, 1 eq) and I-262-5 (60.42 mg, 196.68 μmol, 1.2 eq, 2HCl) in DCM (1 mL) was added T₄P (236.18 mg, 327.80 μmol, 50% purity, 2 eq) and DIEA (84.73 mg, 655.60 μmol, 114.19 μL, 4 eq). The mixture was stirred at 25° C. for 1 h. The reaction mixture was quenched by addition H₂O 1 mL, and then diluted with DCM 1 mL and extracted with DCM 6 mL (2 mL*3). The combined organic layers were dried over anhydrous Na₂SO₄, filtered, and concentrated under reduced pressure to give a residue. The residue was purified by prep-HPLC (column: Waters Xbridge 150*25 mm*5 um; mobile phase: [water (NH4HCO3)-ACN]; gradient: 50%-80% B over 10 min), then concentrated to remove organic solvents and lyophilized to give product. I-262 (65 mg, 78.65 μmol, 47.99% yield, 100% purity) was obtained as a white solid. LCMS: R^{t}=0.451 min, [M+H]⁺=826.5. ¹H NMR (400 MHZ, METHANOL-d4) δ=8.42 (d, J=3.6 Hz, 1H), 7.51-7.45 (m, 2H), 7.42-7.32 (m, 7H), 7.29 (d, J=7.6 Hz, 1H), 4.99-4.96 (m, 1H), 4.29-4.25 (m, 1H), 3.92-3.77 (m, 4H), 3.76-3.62 (m, 4H), 3.61-3.50 (m, 2H), 3.45-3.34 (m, 3H), 3.29-3.20 (m, 1H), 3.06-2.92 (m, 2H), 2.76-2.70 (m, 2H), 2.52-2.49 (m, 2H), 2.47-2.39 (m, 3H), 2.22 (d, J=7.2 Hz, 2H), 2.18-1.99 (m, 4H), 1.75 (d, J=11.8 Hz, 2H), 1.55 (d, J=8.0 Hz, 1H), 1.31-1.18 (m, 5H), 1.16-1.12 (m, 3H). ¹⁹F NMR (377 MHz, METHANOL-d4) δ=−115.17 (br s, 2F). SFC: R^{t}=1.270 min, 1.711 min. HPLC: R^{t}=9.118 min.

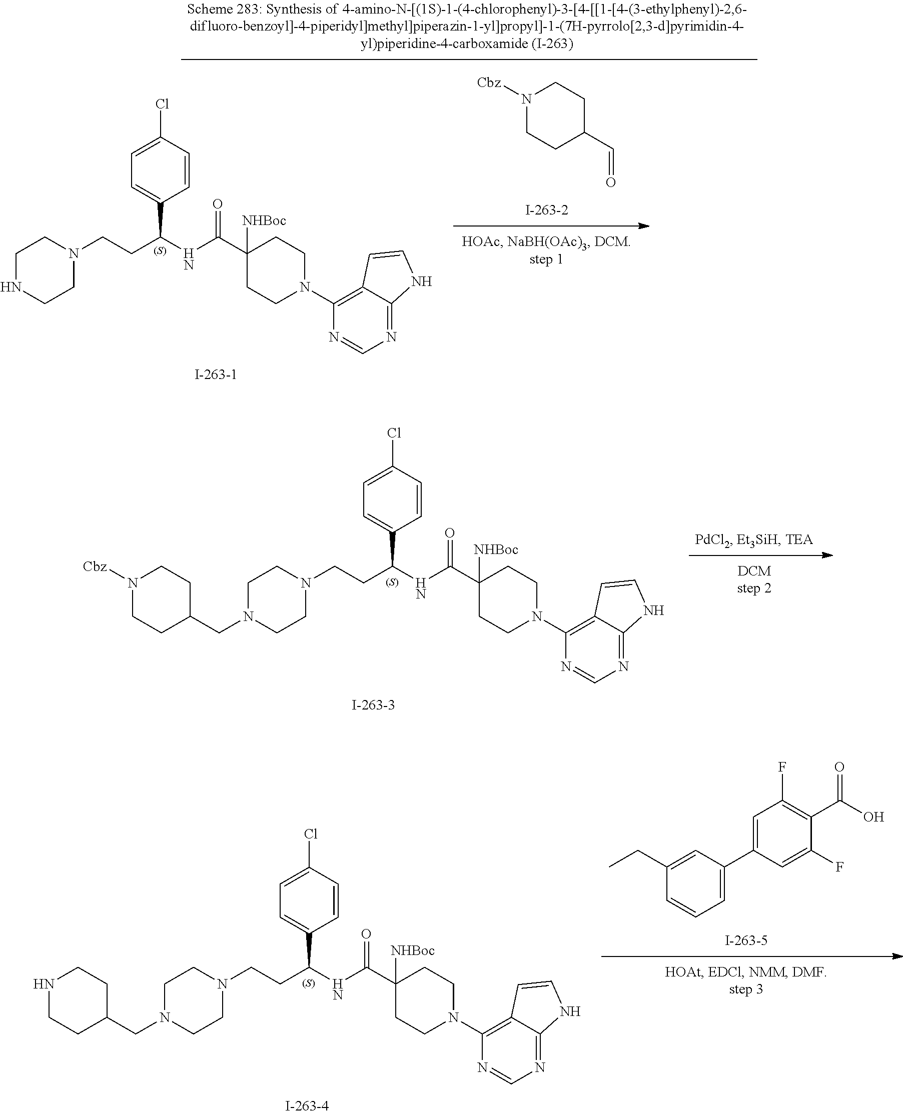

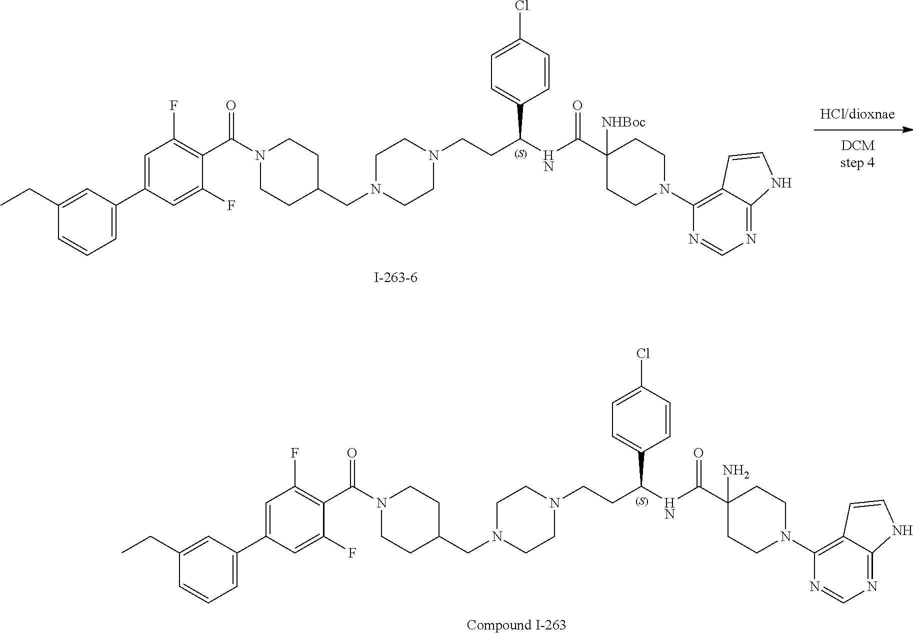

### Step 1: Synthesis of I-263-3

To a solution of I-263-1 (500 mg, 837.31 μmol, 1 eq) and I-263-2 (248.47 mg, 1.00 mmol, 1.2 eq) in DCM (6 mL) was added AcOH (100.56 mg, 1.67 mmol, 95.87 μL, 2 eq) and NaBH(OAc)₃ (887.30 mg, 4.19 mmol, 5 eq), then the mixture was stirred at 20° C. for 5 min. The reaction mixture was added NaHCO₃ about 30 mL to adjust to a pH=7˜8, extracted with DCM 30 mL (15 mL*2). The combined organic layers were concentrated under reduced pressure to give a residue. The residue was purified by flash silica gel chromatography (ISCO®; 20 g Sepa Flash® Silica Flash Column, Eluent of 0˜11% Dichloromethane/Methanol @ 60 mL/min). The eluent was concentrated under reduced pressure to give the product. I-263-3 (650 mg, 761.07 μmol, 90.89% yield, 97% purity) was obtained as a white solid.

LCMS (Method D): R^{t}=0.342 min, [M+H]⁺=828.3. SFC: R^{t}=1.201&1.573 min.

### Step 2: Synthesis of I-263-4

To a solution of I-263-3 (600 mg, 724.25 μmol, 1 eq) in DCM (6 mL) was added PdCl2 (25.69 mg, 144.85 μmol, 0.2 eq) and TEA (146.57 mg, 1.45 mmol, 201.61 μL, 2 eq). The reaction mixture was degassed with N₂. Then a mixture of Et3SiH (336.86 mg, 2.90 mmol, 462.71 μL, 4 eq) in DCM (1 mL) was added dropwise at 10° C., then the mixture was stirred at 25° C. for 2 hours under N₂ atmosphere. The mixture was stirred at 25° C. for 1 hr. The reaction mixture was concentrated under reduced pressure to give a residue. The crude product was purified by reverse-phase column (0.1% FA condition). The eluent was concentrated under reduced pressure to remove ACN and then lyophilized to give the product. I-263-4 (200 mg, 279.42 μmol, 38.58% yield, 97% purity) was obtained as a white solid. LCMS (Method D): R^{t}=0.258 min, [M+H]⁺=694.2. SFC: R^{t}=1.896 min.

### Step 3: Synthesis of I-263-6

To a solution of I-263-4 (50 mg, 67.54 μmol, 1 eq, FA) and I-263-5 (17.71 mg, 67.54 μmol, 1 eq) in DMF (1 mL) was added HOAt (9.19 mg, 67.54 μmol, 9.45 μL, 1 eq), EDCI (38.84 mg, 202.61 μmol, 3 eq) and NMM (34.16 mg, 337.69 μmol, 37.13 μL, 5 eq), then the mixture was stirred at 25° C. for 1 hour. The reaction mixture was diluted with water 40 mL and extracted with DCM 20 mL (10 mL*2). The combined organic layers were concentrated under reduced pressure to give a residue. I-263-6 (150 mg, crude) was obtained as a colorless solid. LCMS (Method D): R^{t}=0.399 min, [M+H]⁺=938.4.

### Step 4: Synthesis of I-263

To a solution of I-263-6 (130.00 mg, 138.51 μmol, 1 eq) in DCM (1.5 mL) was added HCl/dioxane (2 M, 346.28 μL, 5 eq), then the mixture was stirred at 20° C. for 0.5 hour. The reaction mixture was added NaHCO₃ about 20 mL to adjust to a pH=7˜8, extracted with DCM 30 mL (10 mL*3). The combined organic layers were concentrated under reduced pressure to give a residue. The crude product was purified by reversed phase column (0.1% FA condition), then the eluent was concentrated to remove ACN and lyophilized to get the product. I-263 (25.98 mg, 29.37 μmol, 21.21% yield, 100% purity, FA) was obtained as a white solid. LCMS (Method D): R^{t}=0.359 min, [M+H]⁺=838.4. SFC: R^{t}=1.867 min. ¹H NMR (400 MHz, METHANOL-d4) δ=8.44 (s, 1H), 8.15 (s, 1H), 7.52-7.44 (m, 2H), 7.42-7.27 (m, 8H), 7.14 (d, J=3.6 Hz, 1H), 6.64 (d, J=3.6 Hz, 1H), 4.98-4.94 (m, 1H), 4.68 (d, J=12.8 Hz, 1H), 4.60-4.49 (m, 2H), 3.67-3.56 (m, 3H), 3.22-3.13 (m, 1H), 2.94-2.55 (m, 13H), 2.32-2.17 (m, 4H), 2.15-1.99 (m, 2H), 1.88 (d, J=11.6 Hz, 2H), 1.79-1.65 (m, 3H), 1.30-1.26 (m, 3H), 1.23-1.08 (m, 2H), 19F NMR (377 MHz, METHANOL-d4). δ=−115.344, −115.396.

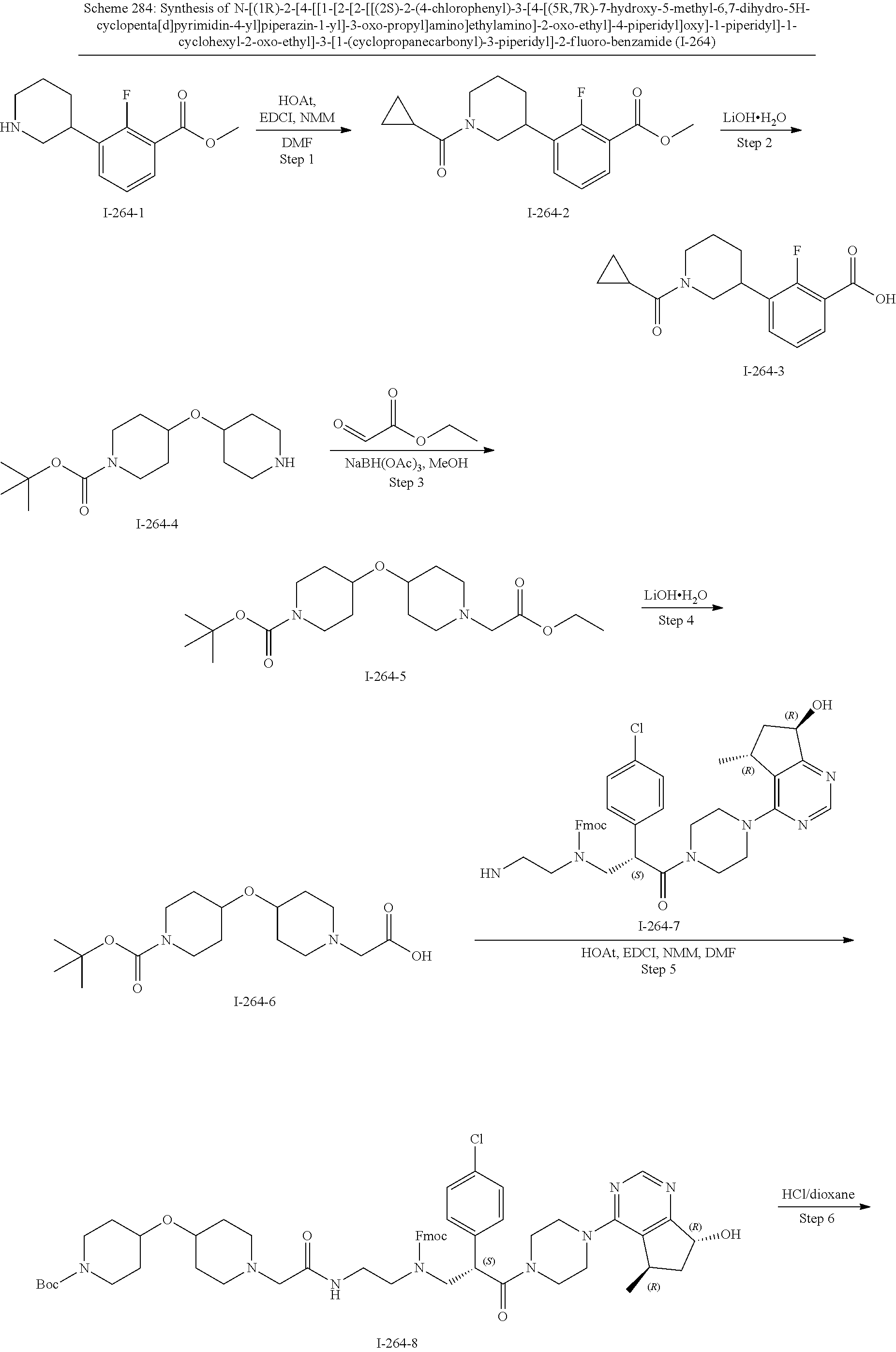

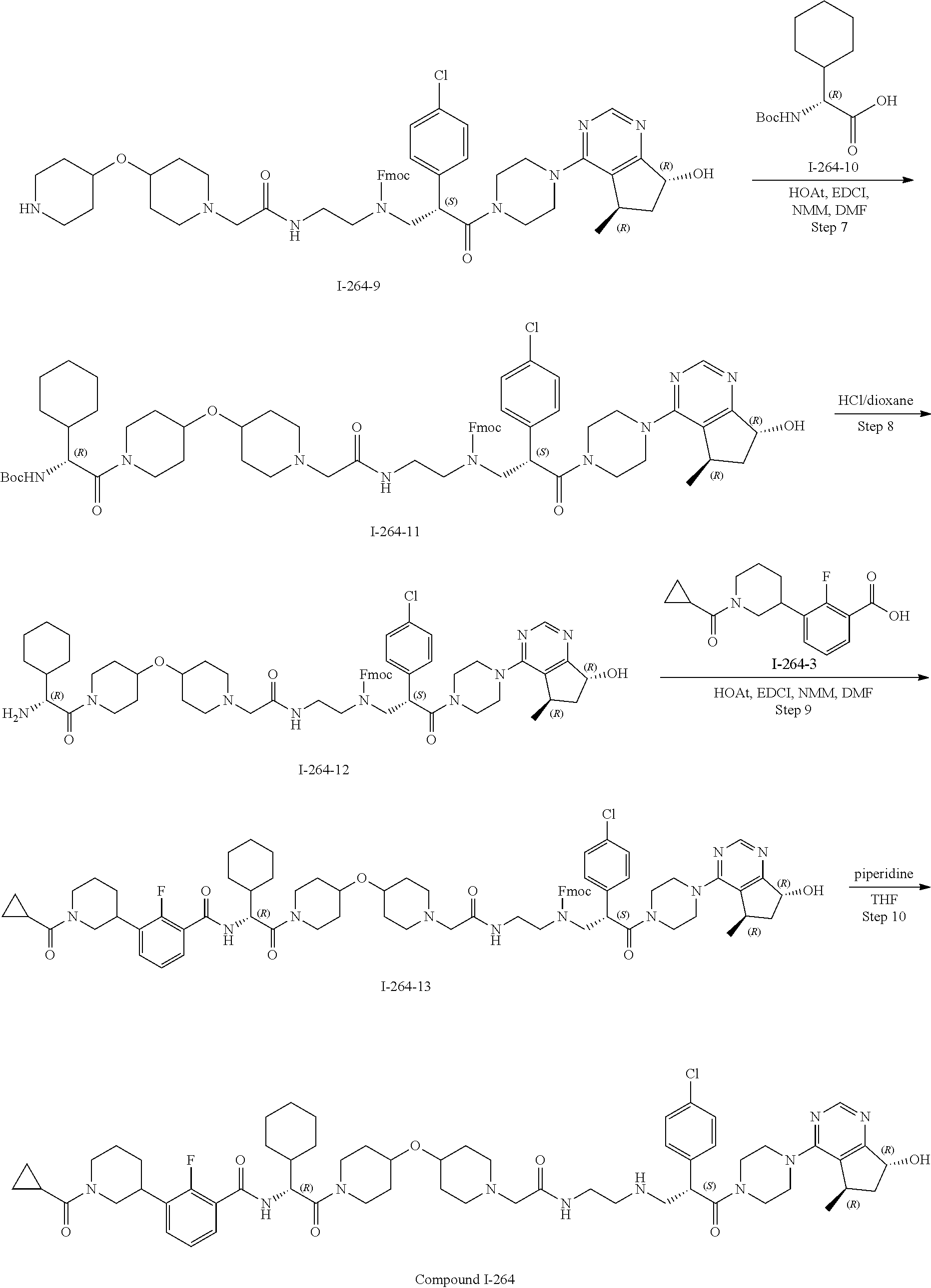

Step 1: Synthesis of I-264-2,

To a mixture of I-264-1 (6 g, 25.29 mmol, 1 eq) and cyclopropanecarboxylic acid (2.18 g, 25.29 mmol, 2.00 mL, 1 eq) in DCM (60 mL) was added HOAt (3.44 g, 25.29 mmol, 3.54 mL, 1 eq), EDCI (14.54 g, 75.86 mmol, 3 eq), NMM (12.79 g, 126.44 mmol, 13.90 mL, 5 eq), then the mixture was stirred at 20° C. for 1 hour. The reaction mixture was diluted with water 100 mL and extracted with EA 60 mL (30 mL*2). The combined organic layers were concentrated under reduced pressure to give a residue. The residue was purified by flash silica gel chromatography ((ISCO®; 40 g SepaFlash® Silica Flash Column, Eluent of 0˜30% Ethylacetate/Petroleum ether gradient (80 mL/min).). The eluent was concentrated under reduced pressure to give I-264-2 (6 g, 19.65 mmol, 77.71% yield) as a white solid. LCMS (Method D): R^{t}=0.391 min, [M+H]⁺=306.3.

### Step 2: Synthesis of I-264-3.

A mixture of I-264-2 (6 g, 19.65 mmol, 1 eq) in THF (40 mL), MeOH (20 mL) and H₂O (10 mL) was added LiOH·H₂O (2.47 g, 58.95 mmol, 3 eq), then the mixture was stirred at 40° C. for 0.5 hour. The reaction mixture was concentrated under reduced pressure to remove THF and MeOH then added 1 N HCl about 40 mL to adjust to a pH=1˜2, filtered, collected the solid, concentrated under reduced pressure to give I-264-3 (5 g, 17.16 mmol, 87.35% yield) as a white solid. LCMS (Method D): R^{t}=0.348 min, [M+H]⁺=291.9.

### Step 3: Synthesis of I-264-5.

To a mixture of I-264-4 (2 g, 7.03 mmol, 1 eq) and ethyl 2-oxoacetate (2.01 g, 9.85 mmol, 1.4 eq) in MeOH (20 mL) was added AcOH (42.23 mg, 703.25 μmol, 40.26 μL, 0.1 eq), stirred at 20° C. for 0.5 hour, then NaBH(OAc)₃ (7.45 g, 35.16 mmol, 5 eq) was added to the mixture and stirred at 20° C. for 0.5 hour. The reaction mixture was diluted with water 50 mL and extracted with DCM 30 mL (10 mL*3). The combined organic layers were concentrated under reduced pressure to give a residue. The residue was purified by flash silica gel chromatography ((ISCO®; 20 g SepaFlash® Silica Flash Column, Eluent of 0˜100% Ethylacetate/Petroleum ether gradient @@ 60 mL/min).). The eluent was concentrated under reduced pressure to give I-264-5 (1.5 g, 4.04 mmol, 57.40% yield, 99.7% purity) as a yellow oil. LCMS: R^{t}=0.273 min, [M+H]⁺=371.2.

### Step 4: Synthesis of I-264-6.

To a mixture of I-264-5 (1 g, 2.70 mmol, 1 eq) in THF (8 mL), MeOH (4 mL) and H₂O (2 mL) was added LiOH·H₂O (339.80 mg, 8.10 mmol, 3 eq), then the mixture was stirred at 20° C. for 1 hour. The reaction mixture was concentrated under reduced pressure to remove THF and MeOH then added 1 N HCl about 40 mL to adjust to a pH=1˜2, filtered, collected the solid, concentrated under reduced pressure to give I-264-6 (400 mg, 1.17 mmol, 43.28% yield) as a white solid. LCMS: R^{t}=0.266 min, [M+H]⁺=343.1.

### Step 5: Synthesis of I-264-8.

To a mixture of I-264-6 (300 mg, 876.09 μmol, 1 eq) and I-264-7 (596.81 mg, 876.09 μmol, 1 eq) in DMF (5 mL) was added HOAt (119.25 mg, 876.09 μmol, 122.55 μL, 1 eq), EDCI (503.84 mg, 2.63 mmol, 3 eq) and NMM (443.07 mg, 4.38 mmol, 481.60 μL, 5 eq), then the mixture was stirred at 20° C. for 1 hour. The reaction mixture was diluted with water 30 mL and extracted with DCM 30 mL (10 mL*3). The combined organic layers were concentrated under reduced pressure to give a residue. The crude product was purified by reverse-phase column (0.1% NH₃·H₂O condition). The eluent was concentrated under reduced pressure to remove MeCN and then lyophilized to give I-264-8 (300 mg, 298.32 μmol, 34.05% yield) as a white solid. LCMS (Method D): R^{t}=0.438 min, [M+H]⁺=1005.7. ¹H NMR (400 MHZ, METHANOL-d4) δ=8.50-8.37 (m, 1H), 7.90-7.79 (m, 2H), 7.72-7.56 (m, 2H), 7.45-7.20 (m, 7H), 6.85 (d, J=8.0 Hz, 1H), 5.00-4.98 (m, 1H), 4.70 (d, J=4.4 Hz, 1H), 4.59-4.50 (m, 1H), 4.25 (d, J=3.2 Hz, 1H), 3.86-3.41 (m, 14H), 3.28-3.20 (m, 3H), 3.16-3.08 (m, 4H), 2.89 (s, 3H), 2.69-2.58 (m, 2H), 2.24-2.03 (m, 4H), 1.83-1.71 (m, 4H), 1.61-1.50 (m, 2H), 1.46 (s, 9H), 1.40 (d, J=4.0 Hz, 2H), 1.17-1.08 (m, 3H).

### Step 6: Synthesis of I-264-9.

To a mixture of I-264-8 (110 mg, 105.56 μmol, 1 eq, HCl) in DCM (1 mL) was added HCl/dioxane (2 M, 1 mL, 18.95 eq), then the mixture was stirred at 20° C. for 10 min. The reaction mixture was concentrated under reduced pressure to give I-264-9 (100 mg, crude, HCl) as a white solid. LCMS (Method D): R^{t}=0.314 min, [M+H]⁺=905.2.

### Step 7: Synthesis of I-264-11.

To a mixture of I-264-9 (90 mg, 95.54 μmol, 1 eq, HCl) and I-264-10 (36.88 mg, 143.32 μmol, 1.5 eq) in DMF (1 mL) was added HOAt (13.00 mg, 95.54 μmol, 13.37 μL, 1 eq), EDCI (54.95 mg, 286.63 μmol, 3 eq) and NMM (48.32 mg, 477.72 μmol, 52.52 μL, 5 eq), then the mixture was stirred at 20° C. for 0.5 h. The reaction mixture was diluted with water 60 mL and extracted with EA 30 mL (10 mL*3). The combined organic layers were concentrated under reduced pressure to give I-264-11 (110 mg, crude) as a white solid. LCMS (Method D): R^{t}=0.415 min, [M+H]⁺=1144.2. SFC: R^{t}=1.397 min.

### Step 8: Synthesis of I-264-12.

To a mixture of I-264-11 (90 mg, 78.61 μmol, 1 eq) in DCM (1 mL) was added HCl/dioxane (2 M, 900.00 μL, 22.90 eq), then the mixture was stirred at 20° C. for 10 min. The reaction mixture was concentrated under reduced pressure to give I-264-12 (90 mg, crude, HCl) as a white solid. LCMS (Method D): R^{t}=0.347 min, [M+H]⁺=1044.3.

### Step 9: Synthesis of I-264-13.

To a mixture of I-264-12 (70 mg, 64.74 μmol, 1 eq, HCl) and I-264-3 (18.86 mg, 64.74 μmol, 1 eq) in DMF (1 mL) was added HOAt (8.81 mg, 64.74 μmol, 9.06 μL, 1 eq), EDCI (37.23 mg, 194.23 μmol, 3 eq) and NMM (32.74 mg, 323.72 μmol, 35.59 μL, 5 eq), then the mixture was stirred at 20° C. for 10 min. The reaction mixture was diluted with water 50 mL and extracted with EA 30 mL (10 mL*3). The combined organic layers were concentrated under reduced pressure to give I-264-13 (80 mg, 60.70 μmol, 93.75% yield) as a yellow solid. LCMS (Method D): R^{t}=0.427 min, [M+H]⁺=1318.2.

### Step 10: Synthesis of I-264.

A mixture of I-264-13 (70 mg, 53.11 μmol, 1 eq) in PIPERIDINE (0.1 mL) and THF (0.8 mL) was stirred at 20° C. for 10 min. The reaction mixture was concentrated under reduced pressure to give a residue. The crude product was purified by reverse-phase column (0.1% FA condition). The eluent was concentrated under reduced pressure to remove MeCN and then lyophilized to give I-264 (32.44 mg, 28.09 μmol, 52.90% yield, 98.88% purity, FA salt) as a white solid. LCMS (Method D): R^{t}=0.350 min, [M+H]⁺=1095.3. SFC: R^{t}=2.548 min, R^{t}=3.515 min. ¹H NMR (400 MHZ, METHANOL-d4), δ=8.42 (d, J=1.6 Hz, 2H), 7.63-7.47 (m, 2H), 7.47-7.41 (m, 2H), 7.39-7.32 (m, 2H), 7.29-7.24 (m, 1H), 5.02-4.91 (m, 2H), 4.64-4.52 (m, 1H), 4.50-4.37 (m, 2H), 4.06-4.03 (m, 1H), 3.94-3.69 (m, 5H), 3.67-3.37 (m, 11H), 3.29-3.11 (m, 8H), 3.10-2.97 (m, 1H), 2.90 (s, 2H), 2.84-2.68 (m, 1H), 2.50 (s, 2H), 2.18-2.09 (m, 1H), 2.09-2.02 (m, 2H), 1.98-1.76 (m, 10H), 1.75-1.61 (m, 6H), 1.57-1.49 (m, 1H), 1.35-1.20 (m, 3H), 1.18-1.06 (m, 5H), 0.95-0.72 (m, 4H). ¹⁹F NMR (376 MHz, METHANOL-d4), δ=−121.663, δ=−121.768.

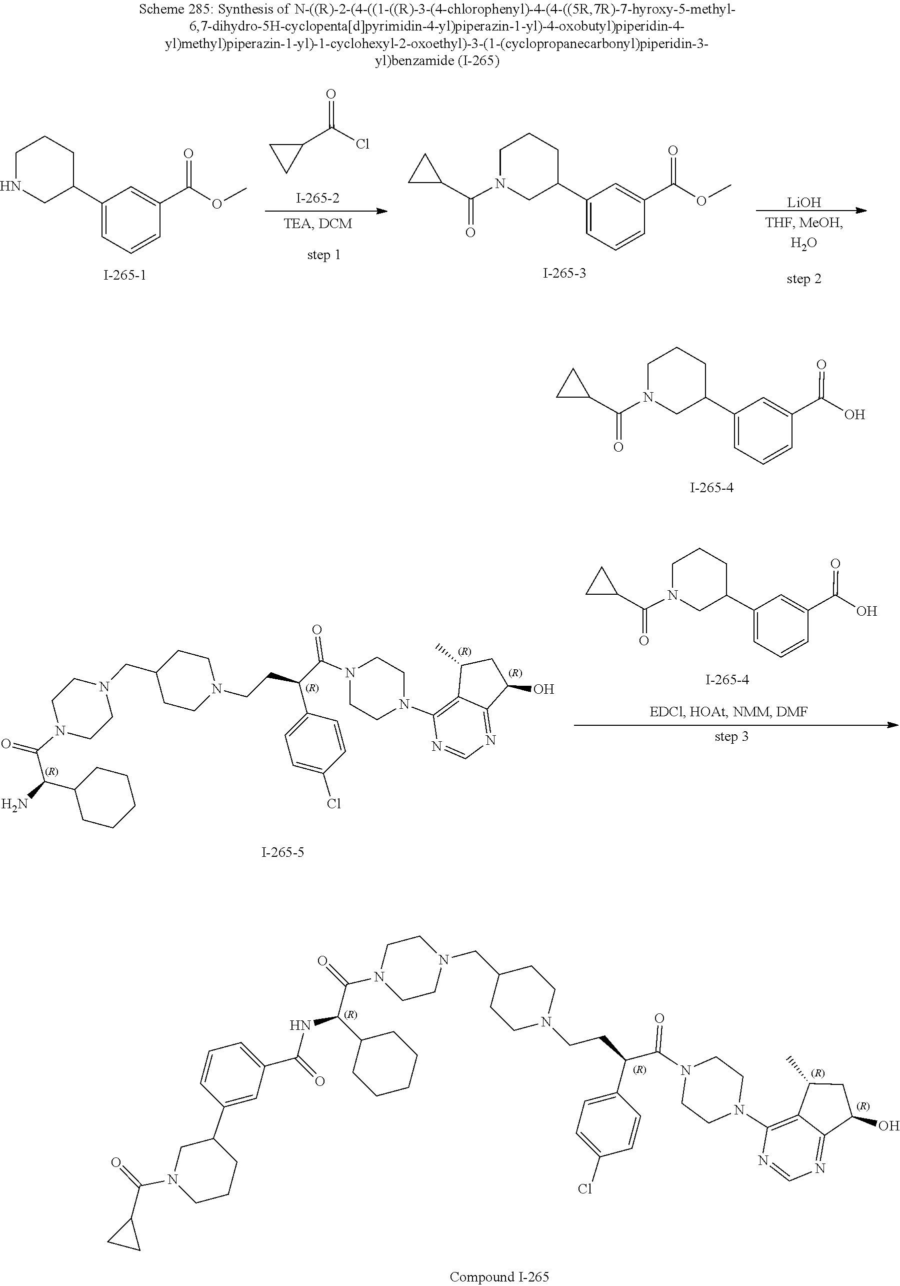

To a solution of 1-2 (190.69 mg, 1.82 mmol, 165.53 μL, 2 eq) and 1-1 (200 mg, 912.08 μmol, 1 eq) in DCM (2 mL) was added TEA (276.88 mg, 2.74 mmol, 380.85 μL, 3 eq). The mixture was stirred at 0° C. for 0.5 hr. The reaction was quenched with H₂O (5 mL), extracted with ethyl acetate 15 mL (5 mL*3). The organic phase was washed with saturated aqueous NaCl (15 mL). Then dried over Na₂SO₄, filtered, and concentrated under reduced pressure to give a residue. The crude product 1-3 (260 mg, 877.67 μmol, 96.23% yield, 97% purity) was used into the next step without further purification. 1-3 (260 mg, 877.67 μmol, 96.23% yield, 97% purity) was obtained as black oil. LCMS: R^{t}=0.517 min, [M+H]⁺=288.2.

### Step 2: Synthesis of 1-4

To a solution of 1-3 (260 mg, 904.81 μmol, 1 eq) in THF (2.6 mL) was added solution of LiOH H₂O (113.91 mg, 2.71 mmol, 3 eq) in H₂O (0.8 mL) and MeOH (2.6 mL). The mixture was stirred at 25° C. for 1 hr. The reaction was quenched with H₂O (15 mL), extracted with ethyl acetate 15 mL (5 mL*3). The organic phase was washed with saturated aqueous NaCl (10 mL). Then dried over Na₂SO₄, filtered, and concentrated under reduced pressure to give a residue. The 1-4 (200 mg, 709.77 μmol, 78.44% yield, 97% purity) was used into the next step without further purification. 1-4 (200 mg, 709.77 μmol, 78.44% yield, 97% purity) as white gum. LCMS: R^{t}=0.450 min, [M+H]⁺=274.2.

### Step 3: Synthesis of I-265

To a solution of 1-5 (70 mg, 90.69 μmol, 1 eq, HCl salt) and 1-4 (24.79 mg, 90.69 μmol, 1 eq) in DMF (1 mL) was added EDCI (52.16 mg, 272.07 μmol, 3 eq), HOAt (12.34 mg, 90.69 μmol, 12.69 μL, 1 eq) and NMM (91.73 mg, 906.90 μmol, 99.71 μL, 10 eq). The mixture was stirred at 25° C. for 1 hr. The reaction was quenched with H₂O (5 mL), extracted with ethyl acetate 9 mL (3 mL*3). The organic phase was washed with saturated aqueous NaCl (10 mL). Then dried over Na₂SO₄, filtered, and concentrated under reduced pressure to give a residue. The product was purified by prep-HPLC (column: Waters Xbridge 150*25 mm*5 um; mobile phase: [water (NH4HCO3)-ACN]; gradient: 45%-75% B over 10 min) and the eluent was concentrated to remove MeCN and then lyophilization to get product. I-265 (24.16 mg, 24.39 μmol, 26.89% yield) was obtained as a white solid. LCMS: R^{t}=0.803 min, [M+H]⁺=990.7. SFC: R^{t}=1.307 min and 1.604 min. ¹H NMR (400 MHZ, METHANOL-d4) δ=8.43 (s, 1H), 7.81-7.65 (m, 2H), 7.54-7.40 (m, 2H), 7.36-7.31 (m, 4H), 4.98-4.95 (m, 1H), 4.57 (d, J=10.4 Hz, 1H), 4.44-4.35 (m, 1H), 4.09-4.06 (m, 1H), 3.85-3.75 (m, 3H), 3.77-3.70 (m, 3H), 3.67 (d, J=5.2 Hz, 2H), 3.62-3.50 (m, 4H), 2.97-2.87 (m, 2H), 2.82-2.67 (m, 2H), 2.51-2.41 (m, 3H), 2.40-2.33 (m, 2H), 2.28 (d, J=8.4 Hz, 2H), 2.21 (d, J=7.2 Hz, 2H), 2.17-1.94 (m, 7H), 1.93-1.83 (m, 5H), 1.77 (br s, 4H), 1.68 (d, J=12.8 Hz, 3H), 1.64-1.50 (m, 2H), 1.32-1.19 (m, 5H), 1.16-1.07 (m, 5H), 0.95-0.71 (m, 5H).

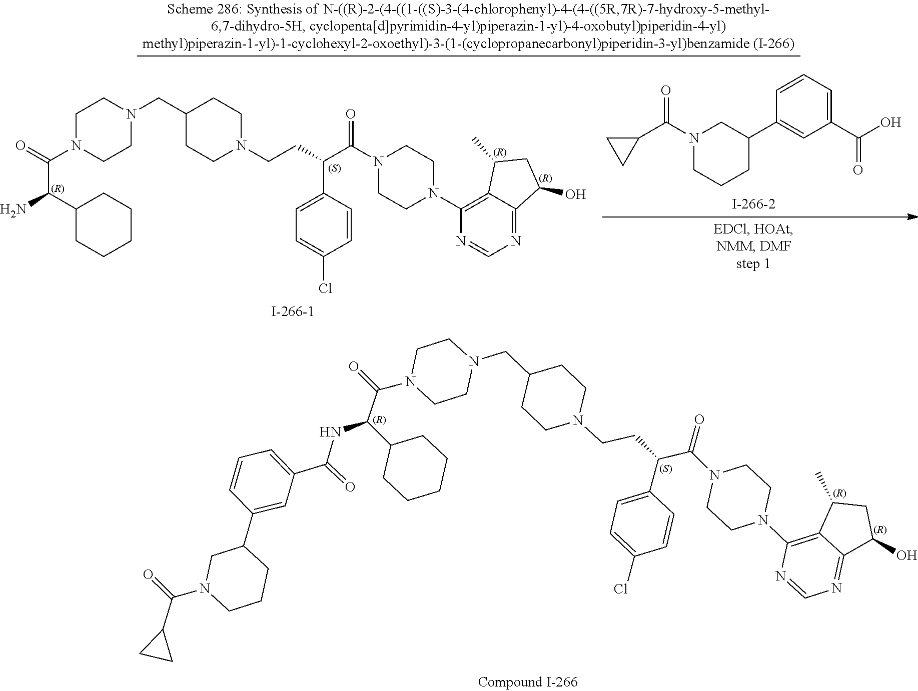

### Step 1: Synthesis of I-266

To a solution of I-266-1 (100 mg, 129.56 μmol, 1 eq, HCl salt) and I-266-2 (35.41 mg, 129.56 μmol, 1 eq) in DMF (1 mL) was added EDCI (74.51 mg, 388.67 μmol, 3 eq), HOAt (17.63 mg, 129.56 μmol, 18.12 μL, 1 eq) and NMM (131.04 mg, 1.30 mmol, 142.44 μL, 10 eq). The mixture was stirred at 25° C. for 1 hr. The reaction was quenched with H₂O (5 mL), extracted with ethyl acetate 9 mL (3 mL+3), The organic phase was washed with saturated aqueous NaCl (10 mL). Then dried over Na₂SO₄, filtered, and concentrated under reduced pressure to give a residue. The product was purified by prep-HPLC (column: Waters Xbridge 150*25 mm*5 um; mobile phase: [water (NH4HCO3)-ACN]; gradient: 50%-80% B over 10 min) and the eluent was concentrated to remove MeCN and then lyophilization to get crude product. I-266 (38.29 mg, 37.49 μmol, 28.94% yield, 97% purity) was obtained as a white solid. LCMS: R^{t}=0.795 min, [M+H]⁺=990.6. SFC: R^{t}=1.777 min and 2.200 min. ¹H NMR (400 MHZ, METHANOL-d4) δ=8.42 (s, 1H), 7.81-7.67 (m, 2H), 7.54-7.40 (m, 2H), 7.38-7.31 (m, 4H), 4.98-4.95 (m, 1H), 4.57 (d, J=11.6 Hz, 1H), 4.45-4.33 (m, 1H), 4.08-4.08 (m, 1H), 3.91-3.80 (m, 2H), 3.76-3.61 (m, 7H), 3.59-3.51 (m, 2H), 3.50-3.41 (m, 1H), 2.95-2.88 (m, 2H), 2.82-2.67 (m, 2H), 2.51-2.40 (m, 3H), 2.39-2.33 (m, 2H), 2.28 (d, J=7.2 Hz, 2H), 2.21 (d, J=6.8 Hz, 2H), 2.17-1.94 (m, 7H), 1.93-1.83 (m, 5H), 1.81-1.74 (m, 4H), 1.68 (d, J=12.8 Hz, 3H), 1.63-1.49 (m, 2H), 1.35-1.19 (m, 5H), 1.16-1.07 (m, 5H), 0.94-0.71 (m, 5H).

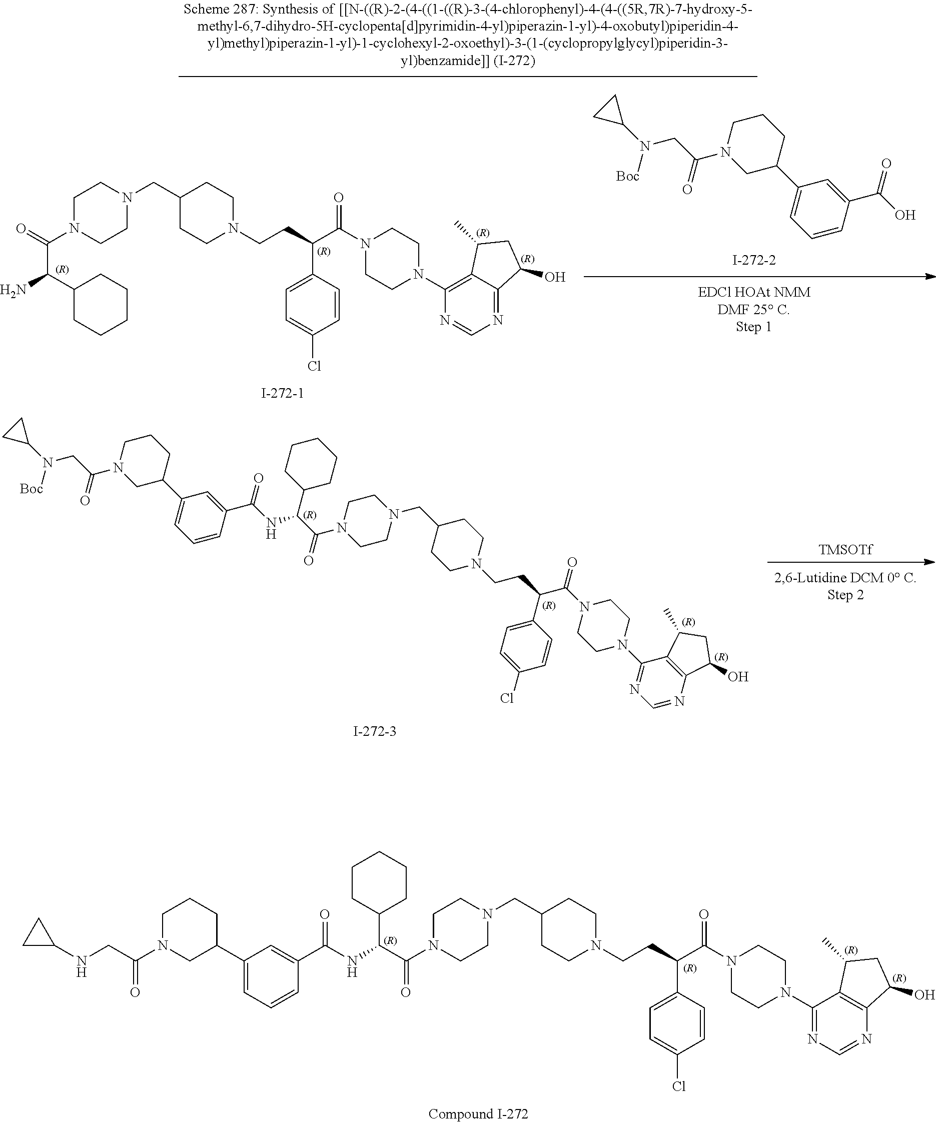

### Step 1: Synthesis of I-272-3

To a solution of I-272-1 (80 mg, 103.65 μmol, 1 eq, HCl), I-272-2 (41.72 mg, 103.65 μmol, 1 eq) in DMF (1 mL) was added EDCI (59.61 mg, 310.94 μmol, 3 eq) and HOAt (14.11 mg, 103.65 μmol, 14.50 μL, 1 eq), NMM (83.87 mg, 829.17 μmol, 91.16 μL, 8 eq). The mixture was stirred at 25° C. for 1 hr. The reaction mixture was quenched by addition water 5 mL at 25° C., and then extracted with EA 15 mL (5 mL*3). The combined organic layers were dried over Na₂SO₄, filtered and concentrated under reduced pressure to give a residue. The residue was purified by flash silica gel chromatography (ISCO®; 12 g SepaFlash® Silica Flash Column, Eluent of 0˜80% MeOH/Ethyl acetate @ 30 mL/min), concentrated under reduced pressure to give product. I-272-3 (100 mg, 87.21 μmol, 84.14% yield, 97.66% purity) was obtained as white solid. LCMS: R^{t}=0.458 min, [M+H]⁺=1119.6

### Step 2: Synthesis of I-272

To a solution of I-272-3 (80 mg, 71.44 μmol, 1 eq) in DCM (1 mL) was added 2,6-lutidine (7.65 mg, 71.44 μmol, 8.32 μL, 1 eq) and TMSOTf (39.69 mg, 178.59 μmol, 32.27 μL, 2.5 eq). The mixture was stirred at 0° C. for 0.5 hr. The reaction mixture was adjust pH=7 with aq. NaHCO₃, and then diluted with EA 3 mL and extracted with EA 3 mL (1 mL*3). The combined organic layers were dried over anhydrous Na₂SO₄, concentrated under reduced pressure to give a residue. The residue was purified by prep-HPLC (column: Phenomenex luna C18 150*25 mm*10 um; mobile phase: [water (FA)-ACN]; gradient: 5%-35% B over 9 min), the target peak was concentrated and lyophilized to afford desired product. I-272 (16 mg, 15.01 μmol, 21.02% yield, 100% purity, FA) was obtained as white solid. LCMS: R^{t}=0.404 min, [M+H]⁺=1019.9. SFC: R^{t}=3.462 min. ¹H NMR (400 MHZ, METHANOL-d4) δ=8.54 (s, 1H), 8.43 (s, 1H), 7.76 (s, 1H), 7.70 (d, J=6.0 Hz, 1H), 7.53-7.41 (m, 2H), 7.41-7.36 (m, 2H), 7.36-7.31 (m, 2H), 4.97 (m, 1H), 4.90 (d, J=8.4 Hz, 1H), 4.63-4.51 (m, 1H), 4.14 (m, 1H), 3.94-3.78 (m, 4H), 3.78-3.40 (m, 12H), 3.28-3.22 (m, 1H), 3.21-3.07 (m, 2H), 2.89-2.68 (m, 4H), 2.66-2.54 (m, 2H), 2.53-2.33 (m, 5H), 2.32-2.22 (m, 3H), 2.18-2.09 (m, 1H), 2.09-2.00 (m, 3H), 1.99-1.83 (m, 6H), 1.77 (s, 3H), 1.69 (d, J=12.4 Hz, 3H), 1.41-1.19 (m, 5H), 1.18-1.05 (m, 5H), 0.58-0.39 (m, 4H). ¹⁹F NMR (376 MHz, METHANOL-d4) δ=−76.85 (s, 1F).

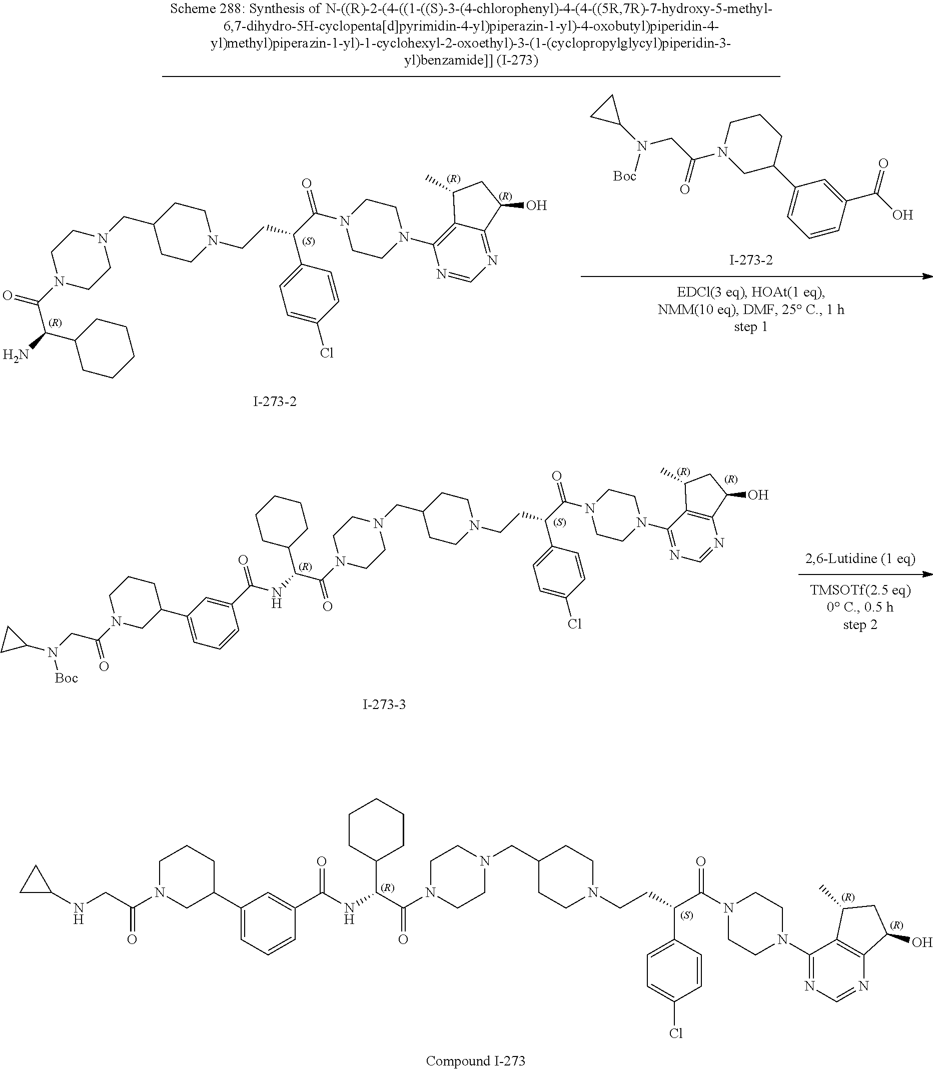

### Step 1: Synthesis of I-273-3

To a solution of I-273-1 (50 mg, 64.78 μmol, 1 eq, HCl salt) and I-273-2 (28.68 mg, 71.26 μmol, 1.1 eq) in DMF (1 mL) was added EDCI (37.25 mg, 194.34 μmol, 3 eq), HOAt (8.82 mg, 64.78 μmol, 9.06 μL, 1 eq) and NMM (65.52 mg, 647.79 μmol, 71.22 μL, 10 eq). The mixture was stirred at 25° C. for 1 h. The reaction mixture was quenched by addition H₂O (1 mL), and then diluted with EA (1 mL) and extracted with EA (2 mL*3). The combined organic layers were washed with brine (2 mL*3), dried over anhydrous Na₂SO₄, filtered and concentrated under reduced pressure to give a residue. The residue was purified by reversed phase (26.8*125 mm, of XB—C18, 20-40 μm, 40 Å) Mobile phase: A for H₂O (0.1% FA v/v) and B for acetonitrile; Gradient: B 0%-100% in 12 min; Flow rate: 60 mL/min; Column temperature: R.T. Wavelength: 220 nm/254 nm), then the eluent was concentrated to remove organic solvents and lyophilized to give I-273-3 (40 mg, 35.72 μmol, 55.14% yield) as a white solid. LCMS: R^{t}=0.485 min, [M+H]⁺=1119.7

### Step 2: Synthesis of I-273

To a solution of I-273-3 (40 mg, 35.72 μmol, 1 eq) in DCM (0.5 mL) was added 2,6-dimethylpyridine (3.83 mg, 35.72 μmol, 4.16 μL, 1 eq) and trimethylsilyl trifluoromethanesulfonate (19.85 mg, 89.30 μmol, 16.14 μL, 2.5 eq). The mixture was stirred at 0° C. for 0.5 h. The reaction mixture was quenched by addition H₂O (0.5 mL), and then diluted with DCM (0.5 mL) and extracted with DCM (1 mL*3). The combined organic layers were dried over anhydrous Na₂SO₄, filtered and concentrated under reduced pressure to give a residue. The residue was purified by prep-HPLC (column: Phenomenex luna C18 150*25 mm*10 um; mobile phase: [water (FA)-ACN]; gradient: 5%-35% B over 9 min), then concentrated to remove organic solvents and lyophilized to give desired product. The product was treated with saturated aq. NaHCO₃, extracted with DCM (1 mL*3). The combined organic layers were dried over anhydrous Na₂SO₄, filtered and concentrated under reduced pressure to give a residue, then the residue was purified by prep-HPLC (column: Phenomenex luna C18 150*25 mm*10 um; mobile phase: [water (FA)-ACN]; gradient: 5%-35% B over 9 min), then concentrated to remove organic solvents and lyophilized to give I-273 (5 mg, 4.90 μmol, 13.73% yield, 100% purity, FA salt) as a white solid. LCMS: R^{t}=0.399 min, [M+H]⁺=1019.7. ¹H NMR (400 MHZ, METHANOL-d4) δ=8.54 (s, 1H), 8.42 (s, 1H), 7.76 (s, 1H), 7.70 (d, J=5.2 Hz, 1H), 7.53-7.42 (m, 2H), 7.40-7.31 (m, 4H), 4.98-4.96 (m, 1H), 4.90 (d, J=8.8 Hz, 1H), 4.61-4.52 (m, 1H), 4.11-4.08 (m, 1H), 3.95-3.50 (m, 15H), 3.50-3.42 (m, 1H), 3.20-3.04 (m, 3H), 2.89-2.69 (m, 2H), 2.64-2.18 (m, 13H), 2.16-2.09 (m, 1H), 2.08-2.03 (m, 2H), 1.97-1.82 (m, 7H), 1.77 (d, J=4.4 Hz, 2H), 1.69 (d, J=12.0 Hz, 3H), 1.36-1.20 (m, 5H), 1.17-1.05 (m, 5H), 0.51-0.37 (m, 4H). SFC: R^{t}=1.909 min, 2.182 min.

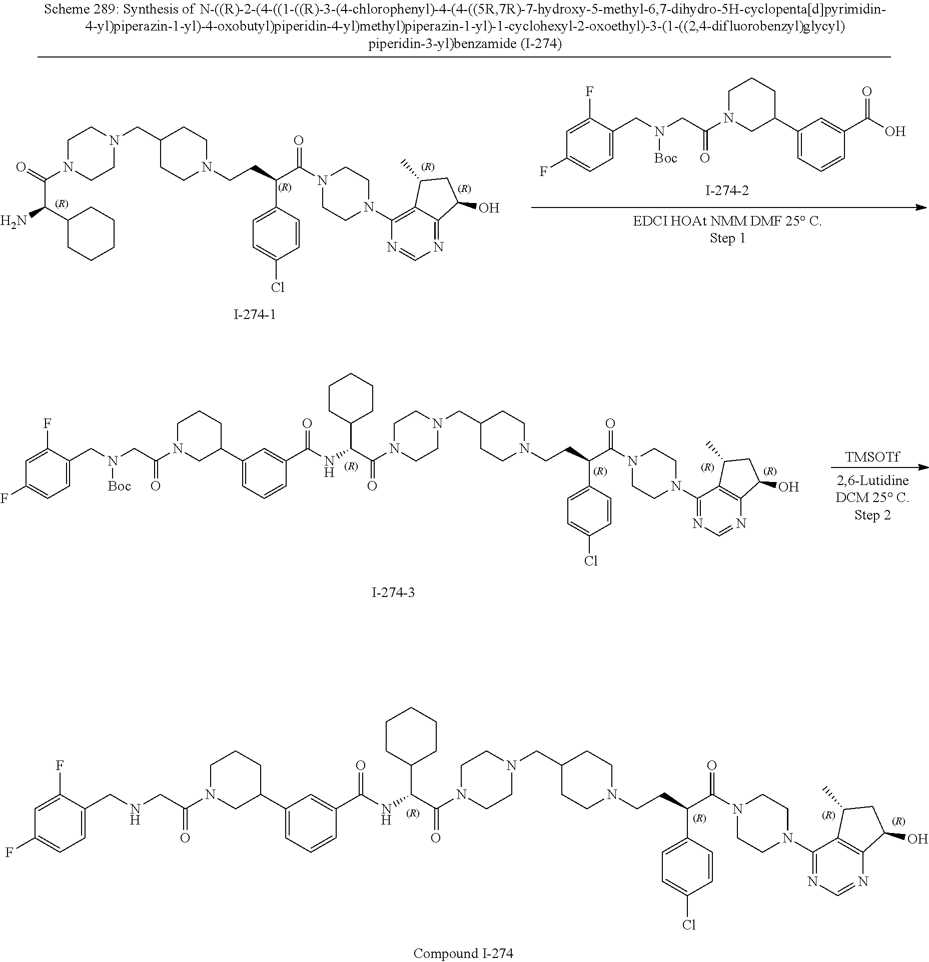

### Step 1: Synthesis of I-274-3

To a solution of I-274-1 (50 mg, 64.78 μmol, 1 eq, HCl salt), I-274-2 (31.65 mg, 64.78 μmol, 1 eq) in DMF (0.5 mL) was added EDCI (37.25 mg, 194.34 μmol, 3 eq) and HOAt (8.82 mg, 64.78 μmol, 9.06 μL, 1 eq), NMM (52.42 mg, 518.23 μmol, 56.98 μL, 8 eq). The mixture was stirred at 25° C. for 1 hr. The reaction mixture was quenched by addition water (5 mL) at 25° C., and then extracted with EA (5 mL*3). The combined organic layers were dried over Na₂SO₄, filtered and concentrated under reduced pressure to give a residue. The residue was purified by flash silica gel chromatography (ISCO®; 12 g SepaFlash® Silica Flash Column, Eluent of 0˜100% Ethyl acetate: Methanol @ 36 mL/min). concentrated under reduced pressure to give I-274-3 (60 mg, 48.43 μmol, 74.76% yield, 97.33% purity) as white solid. LCMS: R^{t}=0.475 min, [M+H]⁺=1205.7

### Step 2: Synthesis of I-274

To a solution of I-274-3 (50 mg, 41.46 μmol, 1 eq) in DCM (0.5 mL) was added 2,6-dimethylpyridine (4.44 mg, 41.46 μmol, 4.83 μL, 1 eq), trimethylsilyl trifluoromethanesulfonate (23.04 mg, 103.66 μmol, 18.73 μL, 2.5 eq). The mixture was stirred at 25° C. for 1 hr. The reaction mixture was adjust pH=7 with aq. NaHCO₃, and then diluted with EA (3 mL) and extracted with EA (1 mL*3). The combined organic layers were dried over anhydrous Na₂SO₄, concentrated under reduced pressure to give a residue. The residue was purified by prep-HPLC (column: Phenomenex Luna C18 150*25 mm*10 um; mobile phase: [water (FA)-ACN]; gradient: 5%-35% B over 9 min), the eluent was concentrated and lyophilized to afford I-274 (20 mg, 18.09 μmol, 43.62% yield, 100% purity, FA salt) as white solid. LCMS: R^{t}=0.429 min, [M+H]⁺=1105.9. SFC: R^{t}=1.095 min, 1.154 min. ¹H NMR (400 MHZ, METHANOL-d4) δ=8.54 (d, J=2.0 Hz, 1H), 8.43 (s, 1H), 7.78-7.67 (m, 2H), 7.51-7.40 (m, 3H), 7.40-7.35 (m, 2H), 7.35-7.29 (m, 2H), 6.99-6.88 (m, 2H), 4.97 (m, 1H), 4.94-4.87 (m, 1H), 4.59-4.50 (m, 1H), 4.17-4.09 (m, 1H), 3.83 (d, J=11.2 Hz, 6H), 3.77-3.64 (m, 5H), 3.63-3.39 (m, 7H), 3.27-3.18 (m, 2H), 3.17-3.07 (m, 2H), 2.84-2.63 (m, 4H), 2.58-2.42 (m, 5H), 2.37 (d, J=6.8 Hz, 2H), 2.24 (d, J=7.6 Hz, 2H), 2.17-2.09 (m, 1H), 2.09-1.98 (m, 3H), 1.96-1.82 (m, 6H), 1.76 (s, 3H), 1.69 (d, J=11.2 Hz, 2H), 1.39-1.21 (m, 5H), 1.17-1.05 (m, 5H). ¹⁹F NMR (376 MHz, METHANOL-d4) δ=−113.20 (d, J=6.8 Hz, 1F), −116.13 (s, 1F).

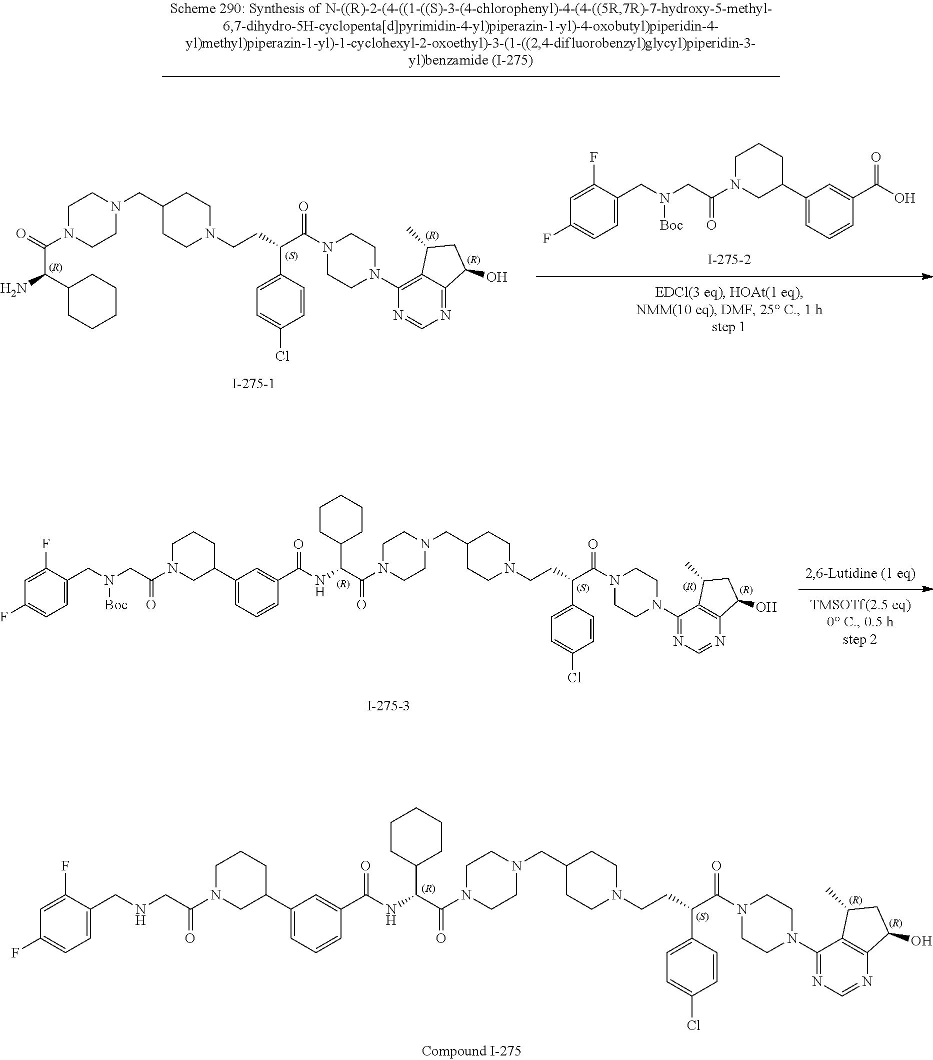

### Step 1: Synthesis of I-275-3

To a solution of I-275-1 (50 mg, 64.78 μmol, 1 eq, HCl salt) and I-275-2 (34.81 mg, 71.26 μmol, 1.1 eq) in DMF (1 mL) was added EDCI (37.25 mg, 194.34 μmol, 3 eq), HOAt (8.82 mg, 64.78 μmol, 9.06 μL, 1 eq) and NMM (65.52 mg, 647.79 μmol, 71.22 μL, 10 eq). The mixture was stirred at 25° C. for 1 h. The reaction mixture was quenched by addition H₂O (1 mL), and then diluted with EA (1 mL) and extracted with EA (2 mL*3). The combined organic layers were washed with brine (2 mL*3), dried over anhydrous Na₂SO₄, filtered and concentrated under reduced pressure to give a residue. The residue was purified by flash silica gel chromatography (ISCO®; 12 g SepaFlash® Silica Flash Column, Eluent of 0˜100% Ethyl acetate: Methanol @ 36 mL/min), then the eluent was concentrated under reduced pressure to give I-275-3 (55 mg, 45.61 μmol, 70.41% yield) as a white solid. LCMS: R^{t}=0.505 min, [M+H]⁺=1205.7

### Step 2: Synthesis of I-275

To a solution of I-275-3 (50 mg, 41.46 μmol, 1 eq) in DCM (0.5 mL) was added 2,6-dimethylpyridine (4.44 mg, 41.46 μmol, 4.83 μL, 1 eq) and trimethylsilyl trifluoromethanesulfonate (23.04 mg, 103.66 μmol, 18.73 μL, 2.5 eq). The mixture was stirred at 0° C. for 0.5 h. The reaction mixture was quenched by addition H₂O (0.5 mL), and then diluted with DCM (0.5 mL) and extracted with DCM (1 mL*3). The combined organic layers were dried over anhydrous Na₂SO₄, filtered and concentrated under reduced pressure to give a residue. The residue was purified by prep-HPLC (column: Phenomenex luna C18 150*25 mm*10 um; mobile phase: [water (FA)-ACN]; gradient: 5%-35% B over 9 min), then the eluent was concentrated to remove organic solvents and lyophilized to give product. The product was treated with saturated aq. NaHCO₃, extracted with DCM (1 mL*3). The combined organic layers were dried over anhydrous Na₂SO₄, filtered and concentrated under reduced pressure to give a residue, then the residue was purified by prep-HPLC (column: Phenomenex luna C18 150*25 mm*10 um; mobile phase: [water (FA)-ACN]; gradient: 8%-38% B over 9 min), then the eluent was concentrated to remove organic solvents and lyophilized to give I-275 (7 mg, 6.08 μmol, 14.66% yield, 100% purity; FA salt) as a white solid. LCMS: R^{t}=0.417 min, [M+H]⁺=1105.6. SFC: R^{t}=1.896 min, 2.097 min. ¹H NMR (400 MHZ, METHANOL-d4) δ=8.55-8.47 (m, 1H), 8.41 (s, 1H), 7.79-7.68 (m, 2H), 7.51-7.38 (m, 5H), 7.36-7.30 (m, 2H), 7.03-6.91 (m, 2H), 4.99-4.96 (m, 1H), 4.92-4.89 (mz, 1H), 4.59-4.48 (m, 1H), 4.14 (s, 1H), 3.94-3.77 (m, 6H), 3.75-3.39 (m, 14H), 3.24-3.11 (m, 2H), 2.99 (d, J=4.4 Hz, 1H), 2.92-2.70 (m, 5H), 2.54-2.37 (m, 5H), 2.26 (d, J=6.8 Hz, 2H), 2.15-1.97 (m, 6H), 1.94-1.81 (m, 5H), 1.78 (d, J=10.4 Hz, 2H), 1.69 (d, J=11.2 Hz, 2H), 1.46-1.35 (m, 2H), 1.33-1.20 (m, 3H), 1.16-1.05 (m, 5H). ¹⁹F NMR (377 MHz, METHANOL-d4) δ=−80.44 (m, 3F), −112.56 (s, 1F), −115.74 (s, 1F). ¹⁹F NMR (376 MHz, METHANOL-d4) δ=−112.47 (m, 1F), −115.65 (m, 1F).

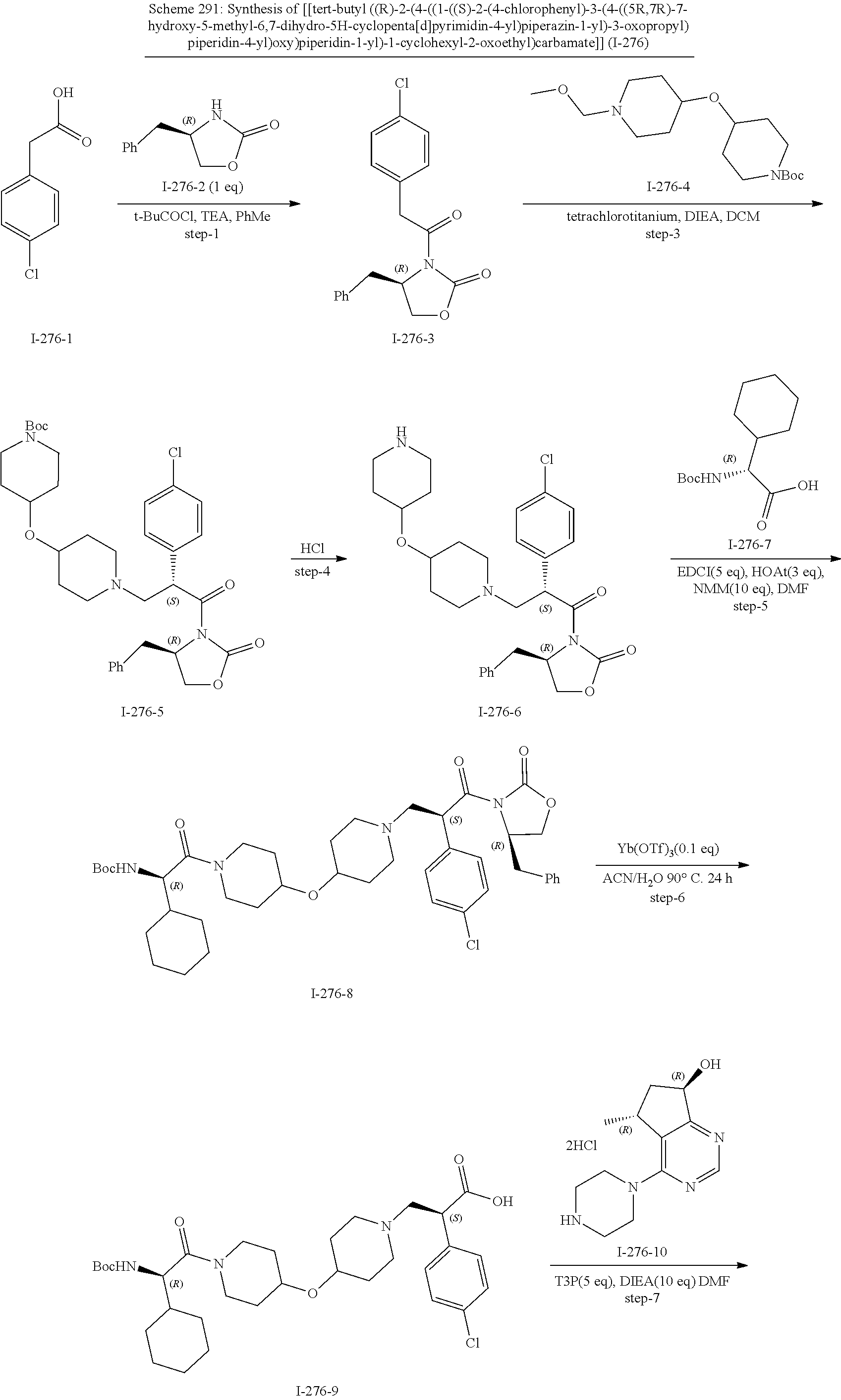

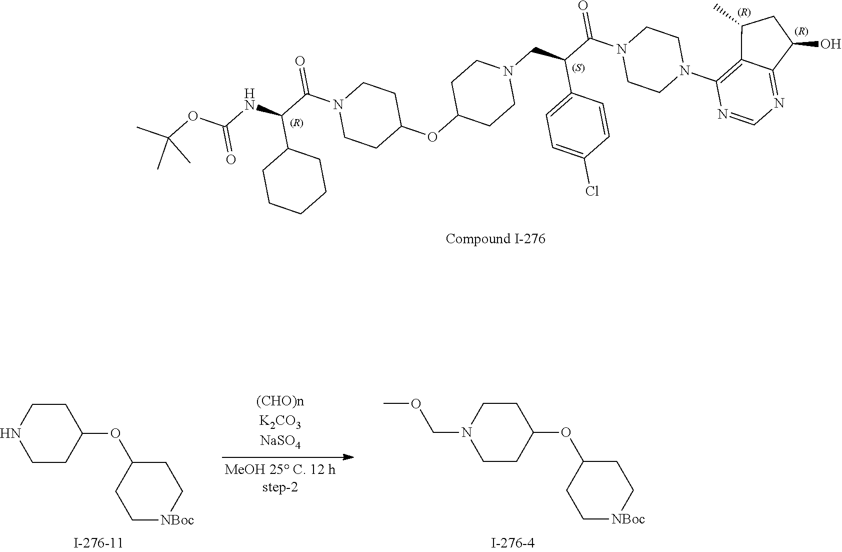

### Step 1: Synthesis of I-276-3

To a solution of I-276-1 (5 g, 29.31 mmol, 1 eq) and I-276-2 (2.60 g, 14.65 mmol, 0.5 eq) in toluene (50 mL) was added TEA (5.93 g, 58.62 mmol, 8.16 mL, 2 eq). The mixture was stirred at 80° C. Then 2, 2-dimethylpropanoyl chloride (3.53 g, 29.31 mmol, 3.61 mL, 1 eq) was added in. The mixture was stirred at 110° C. for 1 hr under N₂ atmosphere. The reaction mixture was filtered, the filter cake was washed by toluene (50 mL*3). The filtrate was combined and concentrated to afford a residue. The residue was purified by flash silica gel chromatography (ISCO®; 120 g SepaFlash® Silica Flash Column, Eluent of 0˜40% Ethylacetate/Petroleum ether gradient @ 100 mL/min). The eluent was concentrated to afford I-276-1 (9.0 g, 27.29 mmol, 93.11% yield) as a yellow solid.

### Step 2: Synthesis of I-276-4

To a solution of I-276-11 (527.89 mg, 17.58 mmol, 484.31 μL, 1 eq) in MeOH (50 mL) was added K₂CO₃ (4.86 g, 35.16 mmol, 2 eq) and Na₂SO₄ (4.99 g, 35.16 mmol, 3.57 mL, 2 eq). The mixture was stirred at 25° C. for 12 hr. The reaction mixture was filtered and concentrated under reduced pressure give I-276-4 (6.0 g, crude) as a white oil, and it was used into the next step without further purification. ¹H NMR (400 MHZ, METHANOL-d4) δ=4.00 (s, 2H), 3.80-3.70 (m, 2H), 3.68-3.62 (m, 1H), 3.53-3.47 (m, 1H), 3.35 (s, 3H), 3.18-3.05 (m, 2H), 2.93-2.82 (m, 2H), 2.61-2.48 (m, 2H), 1.92-1.74 (m, 4H), 1.62-1.50 (m, 2H), 1.45 (s, 10H), 1.44-1.35 (m, 1H).

### Step 3: Synthesis of I-276-5

To a solution of I-276-3 (1 g, 3.03 mmol, 1 eq) in DCM (25 mL) was added tetrachlorotitanium (1 M, 3.18 mL, 1.05 eq) and DIEA (431.10 mg, 3.34 mmol, 581.00 μL, 1.1 eq) under N₂. The mixture was stirred at −70° C. for 0.5 hours. Then I-276-4 (1.29 g, 3.94 mmol, 1.3 eq) was added in. The mixture was stirred at 25° C. for 1 hours. The reaction was diluted with Sat. NH₄Cl (50 mL), extracted with ethyl acetate (100 mL*3), the organic phase was washed with saturated aqueous NaCl (100 mL). Then dried over

[Na₂SO₄], filtered and concentrated under reduced pressure to give a residue. The residue was purified by flash silica gel chromatography (ISCO®; 40 g SepaFlash® Silica Flash Column, Eluent of 0˜100% Ethyl acetate/MeOH gradient @ 60 mL/min). The eluent was concentrated to afford I-276-5 (1.2 g, 1.92 mmol, 63.20% yield) as a white solid. LCMS: R^{t}=0.548 min, [M+H]⁺=626.3

### Step 4: Synthesis of I-276-6

To a solution of I-276-5 (2 g, 3.19 mmol, 1 eq) in DCM (20 mL) was added HCl/dioxane (2 M, 10.00 mL, 6.26 eq). The mixture was stirred at 25° C. for 0.5 hr. The reaction was filtered and concentrated under reduced pressure to give I-276-6 (2.0 g, crude, HCl salt) as a white solid, and it was used into the next step without further purification. LCMS: R^{t}=0.438 min, [M+H]⁺=526.3

### Step 5: Synthesis of I-276-8

To a solution of I-276-6 (2 g, 3.56 mmol, 1 eq, HCl salt) and I-276-7 (1.01 g, 3.91 mmol, 1.1 eq) in DCM (20 mL) was added EDCI (2.04 g, 10.67 mmol, 3 eq), NMM (1.80 g, 17.78 mmol, 1.95 mL, 5 eq) and HOAt (725.89 mg, 5.33 mmol, 746.03 μL, 1.5 eq). The mixture was stirred at 25° C. for 1 hr. The reaction was diluted with H₂O (20 mL), extracted with ethyl acetate (50 mL*3), the organic phase was washed with saturated aqueous NaCl (50 mL). Then dried over [Na₂SO₄], filtered and concentrated under reduced pressure to give a residue. The residue was purified by flash silica gel chromatography (ISCO®; 80 g SepaFlash® Silica Flash Column, Eluent of 0˜60% Ethyl acetate/MeOH gradient (100 mL/min). The eluent was concentrated to afford I-276-8 (2.0 g, 2.61 mmol, 73.50% yield) as a white solid. LCMS: R^{t}=0.897 min, [M+H]⁺=765.5. SFC: R^{t}=1.165 min. ¹H NMR (400 MHZ, CHLOROFORM-d) δ=7.43-7.36 (m, 2H), 7.35-7.31 (m, 2H), 7.25-7.16 (m, 3H), 6.98-6.97 (m, 2H), 5.43-5.33 (m, 1H), 5.30 (br s, 1H), 4.77 (br s, 1H), 4.51-4.43 (m, 1H), 4.24 (br t, J=8.8 Hz, 1H), 3.99-3.84 (m, 1H), 3.81-3.71 (m, 1H), 3.65-3.64 (m, 1H), 3.46-3.18 (m, 5H), 3.06-3.04 (m, 1H), 2.95-2.88 (m, 1H), 2.77-2.75 (m, 1H), 2.67-2.62 (m, 1H), 2.48 (br d, J=8.0 Hz, 1H), 2.31 (br t, J=8.8 Hz, 1H), 2.20-2.08 (m, 1H), 1.89-1.69 (m, 7H), 1.65 (br d, J=10.0 Hz, 3H), 1.53 (br d, J=8.4 Hz, 3H), 1.43 (s, 9H), 1.25-0.94 (m, 6H).

### Step 6: Synthesis of I-276-9

To a solution of I-276-8 (200 mg, 261.31 μmol, 1 eq) in ACN (2 mL) and H₂O (0.5 mL) was added Yb(OTf)₃ (16.21 mg, 26.13 μmol, 0.1 eq). The mixture was stirred at 65° C. for 2 hr. The reaction mixture was filtered give a residue. The crude product was purified by reversed-phase HPLC (0.1% FA condition). The eluent was concentrated under reduced pressure to remove ACN, then lyophilized to afford I-276-9 (80 mg, 131.97 μmol, 50.50% yield, 100% purity) as a white solid. LCMS: R^{t}=0.510 min, [M+H]⁺=606.4. SFC: R^{t}=0.766 min.

### Step 7: Synthesis of I-276

To a solution of I-276-9 (80 mg, 131.97 μmol, 1 eq) and I-276-10 (60.82 mg, 197.96 μmol, 1.5 eq, 2HCl salt) in DCM (1 mL) was added T₄P (475.44 mg, 659.86 μmol, 50% purity, 5 eq) and DIEA (170.56 mg, 1.32 mmol, 229.87 μL, 10 eq). The mixture was stirred at 25° C. for 1 hr. The reaction was diluted with H₂O (5 mL), extracted with DCM (5 mL*3), the organic phase was washed with saturated aqueous NaCl (5 mL). Then dried over [Na₂SO₄], filtered and concentrated under reduced pressure to give a residue. The residue was purified by prep-HPLC (column: Waters Xbridge 150*25 mm*5 um; mobile phase: [water (NH₄HCO₃)-ACN]; gradient: 48%-78% B over 10 min). The eluent was concentrated under reduced pressure to remove ACN, then lyophilized to afford product. The product was re-purified by prep-HPLC (column: Phenomenex luna C18 150*25 mm*10 um; mobile phase: [water (FA)-ACN]; gradient: 15%-45% B over 15 min). The eluent was concentrated under reduced pressure to remove ACN, then lyophilized to afford I-276 (12 mg, 13.82 μmol, 10.47% yield, 100% purity, FA salt) as a white solid. LCMS: R^{t}=0.871 min, [M+H]⁺=822.5. SFC: R^{t}=1.485 min. ¹H NMR (400 MHZ, METHANOL-d4) δ=8.55-8.49 (m, 1H), 8.43 (s, 1H), 7.39-7.25 (m, 4H), 4.99-4.95 (m, 1H), 4.40-4.33 (m, 1H), 4.30-4.27 (m, 1H), 4.02-3.73 (m, 6H), 3.72-3.66 (m, 2H), 3.65-3.50 (m, 4H), 3.43-3.34 (m, 3H), 3.27-3.15 (m, 1H), 2.97-2.79 (m, 2H), 2.56-2.47 (m, 1H), 2.46-2.28 (m, 2H), 2.18-2.04 (m, 2H), 1.97-1.82 (m, 4H), 1.81-1.69 (m, 3H), 1.69-1.53 (m, 6H), 1.43 (s, 9H), 1.31-1.18 (m, 3H), 1.16 (d, J=6.8 Hz, 3H), 1.09-0.96 (m, 2H).

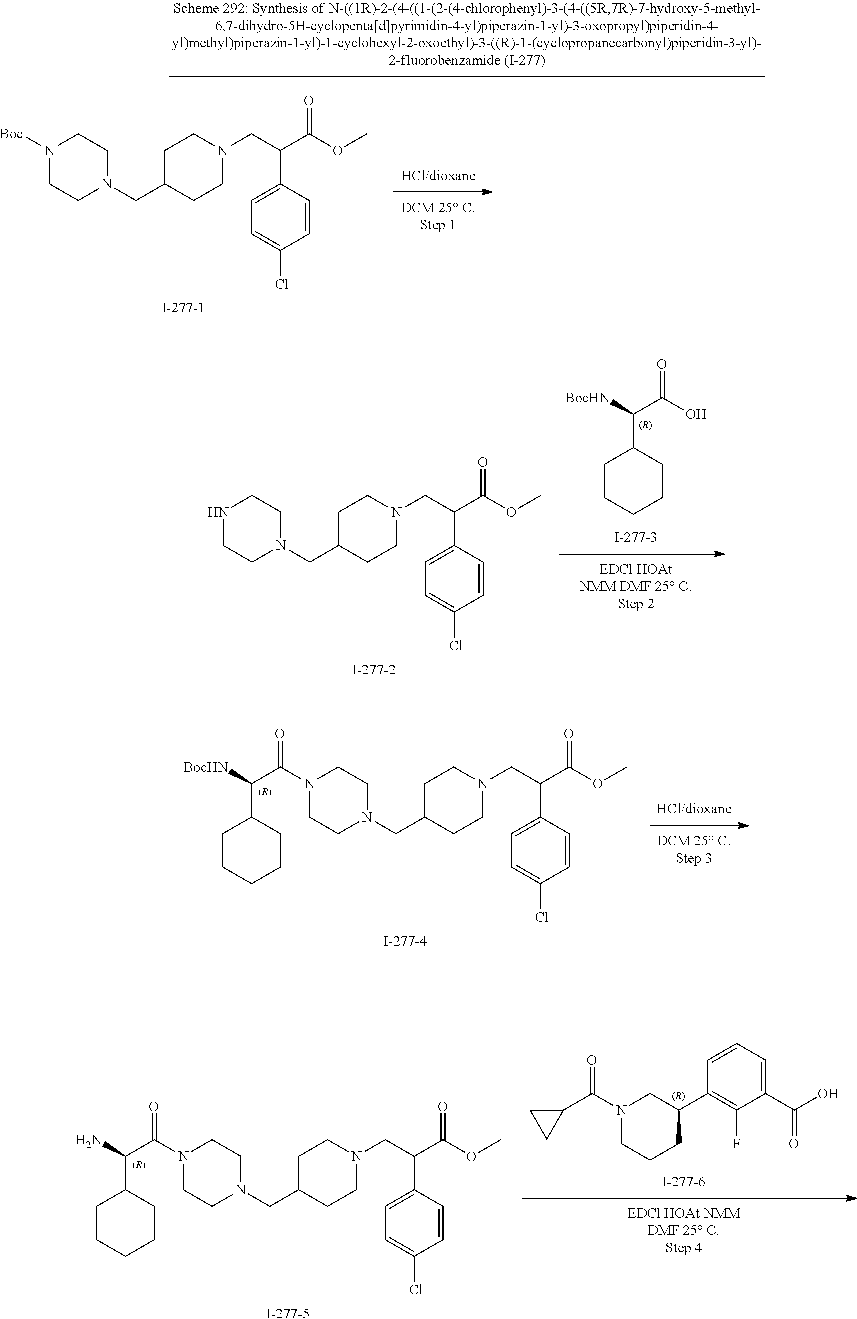

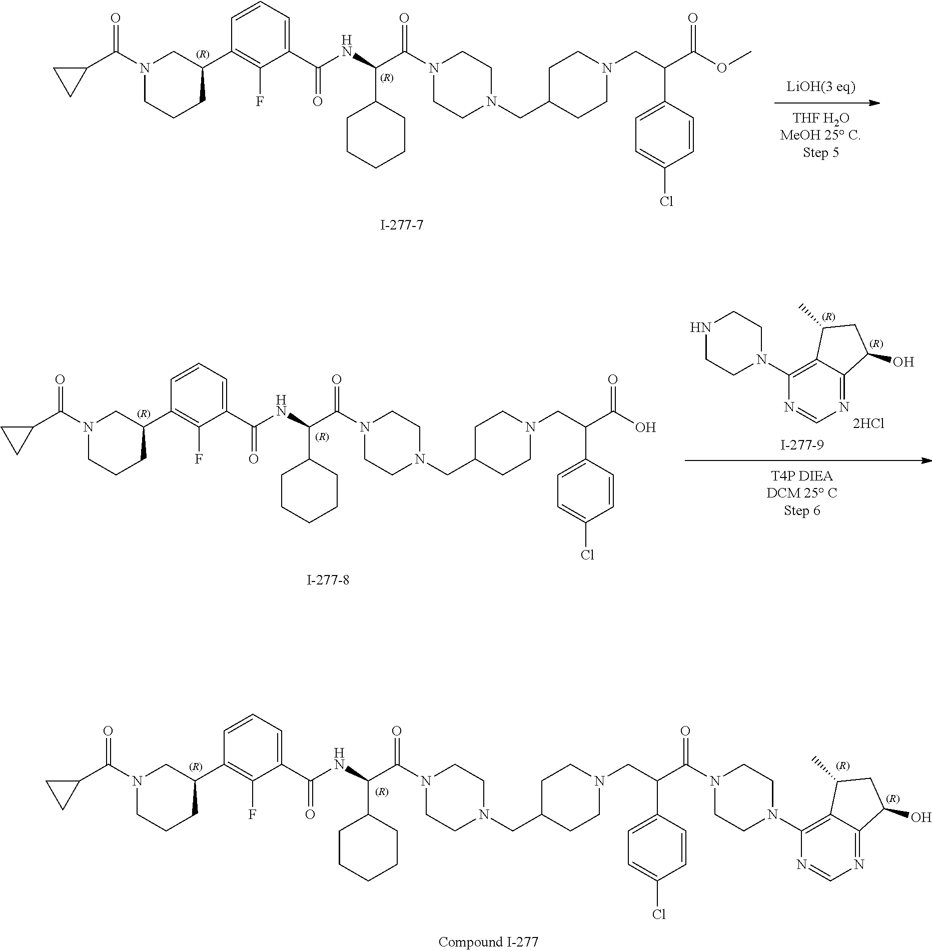

### Step 1: Synthesis of I-277-2

To a solution of I-277-1 (3 g, 6.25 mmol, 1 eq) in DCM (30 mL) was added HCl/dioxane (2 M, 30.00 mL, 9.60 eq). The mixture was stirred at 25° C. for 1 hr. The reaction was filtered and concentrated under reduced pressure to give I-277-2 (2.2 g, 4.85 mmol, 77.53% yield, 91.70% purity; HCl salt) as a white solid. LCMS: R^{t}=0.345 min, [M+H]⁺=380.2

### Step 2: Synthesis of I-277-4

To a solution of I-277-2 (2.2 g, 5.28 mmol, 1 eq, HCl salt) and I-277-3 (1.63 g, 6.34 mmol, 1.2 eq) in DMF (20 mL) was added EDCI (3.04 g, 15.85 mmol, 3 eq) and HOAt (719.15 mg, 5.28 mmol, 739.11 μL, 1 eq), NMM (4.28 g, 42.27 mmol, 4.65 mL, 8 eq). The mixture was stirred at 25° C. for 1 hr. The reaction mixture was quenched by addition water (2 mL) at 25° C., and then extracted with EA (2 mL*3). Then dried over Na₂SO₄, filtered and concentrated under reduced pressure to give a residue. The residue was purified by reversed phase (26.8*125 mm, 120 g of XB—C18, 20-40 μm, 120 Å) Mobile phase: A for H₂O (0.1% FV v/v) and B for acetonitrile; Gradient: B 30%-50% in 30 min: Flow rate: 60 ml/min; Column temperature: R.T. Wavelength: 220 nm/254 nm), then the eluent was concentrated to remove organic solvents and then lyophilization to get I-277-4 (2.1 g, 2.84 mmol, 53.83% yield, 83.86% purity) as a white solid. LCMS: R^{t}=0.897 min, [M+H]⁺=619.5

### Step 3: Synthesis of I-277-5

To a solution of I-277-4 (2 g, 3.23 mmol, 1 eq) in DCM (20 mL) was added HCl/dioxane (2 M, 20.00 mL, 12.38 eq). The mixture was stirred at 25° C. for 1 hr. The reaction mixture was concentrated under reduced pressure to give I-277-5 (1.6 g, 2.52 mmol, 77.97% yield, 87.44% purity, HCl salt) as a white solid. LCMS: R^{t}=0.717 min, [M+H]⁺=519.4

### Step 4: Synthesis of I-277-7

To a solution of I-277-5 (100 mg, 192.63 μmol, 1 eq) and I-277-6 (67.34 mg, 231.16 μmol, 1.2 eq) in DMF (1 mL) was added EDCI (110.78 mg, 577.90 μmol, 3 eq) and HOAt (26.22 mg, 192.63 μmol, 26.95 μL, 1 eq), NMM (155.88 mg, 1.54 mmol, 169.43 μL, 8 eq). The mixture was stirred at 25° C. for 1 hr. The reaction mixture was quenched by addition water (5 mL) at 25° C., and then extracted with EA (5 mL*3). The combined organic layers were dried over Na₂SO₄, filtered and concentrated under reduced pressure to give a residue. The residue was purified by reversed phase (26.8*125 mm, 80 g of XB—C18, 20-40 μm, 120 Å) Mobile phase: A for H₂O (0.1% FA v/v) and B for acetonitrile; Gradient: B 30%-50% in 30 min: Flow rate: 60 ml/min; Column temperature: R.T. Wavelength: 220 nm/254 nm), the eluent was concentrated and lyophilized to afford I-277-7 (100 mg, 109.16 μmol, 56.67% yield, 86.5% purity) as white solid. LCMS: R^{t}=0.483 min, [M+H]⁺=792.4.

### Step 5: Synthesis of I-277-8

To a solution of I-277-7 (100 mg, 126.20 μmol, 1 eq) in THF (0.4 mL), MeOH (0.2 mL) was added LiOH·H₂O (15.89 mg, 378.59 μmol, 3 eq) and H₂O (0.4 mL). The mixture was stirred at 25° C. for 1 hr. The reaction mixture was adjust pH=6 with aq. HCl (1 M), and then diluted with EA (10 mL) and extracted with EA (10 mL*3). The combined organic layers were dried over anhydrous Na₂SO₄, concentrated under reduced pressure to give I-277-8 (90 mg, 100.86 μmol, 79.92% yield, 87.23% purity) as colorless gum. LCMS: R^{t}=0.461 min, [M+H]⁺=778.4. SFC: R^{t}=1.308 min.

### Step 6: Synthesis of I-277

To a solution of I-277-8 (60 mg, 77.08 μmol, 1 eq) and I-277-9 (28.42 mg, 92.50 μmol, 1.2 eq, 2HCl salt) in DMF (1 mL) was added DIEA (29.89 mg, 231.25 μmol, 40.28 μL, 3 eq) and T₄P (83.31 mg, 231.25 μmol, 3 eq). The mixture was stirred at 25° C. for 1 hr. The reaction mixture was quenched by addition water (5 mL) at 25° C., and then extracted with EA (5 mL*3). The combined organic layers were dried over Na₂SO₄, filtered and concentrated under reduced pressure to give a residue. The residue was purified by prep-HPLC (column: Waters Xbridge 150*25 mm*5 um; mobile phase: [water (NH₄HCO₃)-ACN]; gradient: 50%-80% B over 10 min), the eluent was concentrated and lyophilized to afford I-277 (8 mg, 7.69 μmol, 9.97% yield, 95.57% purity) as white solid. LCMS: R^{t}=0.464 min, [M+H]⁺=994.7. SFC: R^{t}=1.787 min. ¹H NMR (400 MHZ, METHANOL-d4) δ=8.43 (d, J=3.6 Hz, 1H), 7.60-7.46 (m, 2H), 7.38-7.30 (m, 4H), 7.26 (m, 1H), 4.98 (m, 1H), 4.95-4.91 (m, 1H), 4.62-4.53 (m, 1H), 4.47-4.36 (m, 1H), 4.28 (m, 1H), 3.94-3.78 (m, 3H), 3.77-3.61 (m, 6H), 3.60-3.47 (m, 3H), 3.27-3.12 (m, 2H), 3.12-2.89 (m, 3H), 2.87-2.71 (m, 1H), 2.54-2.34 (m, 5H), 2.28-1.98 (m, 8H), 1.97-1.65 (m, 11H), 1.64-1.47 (m, 2H), 1.33-1.11 (m, 10H), 0.95-0.79 (m, 4H). ¹⁹F NMR (377 MHz, METHANOL-d4) δ=−121.70 (m, 1F).

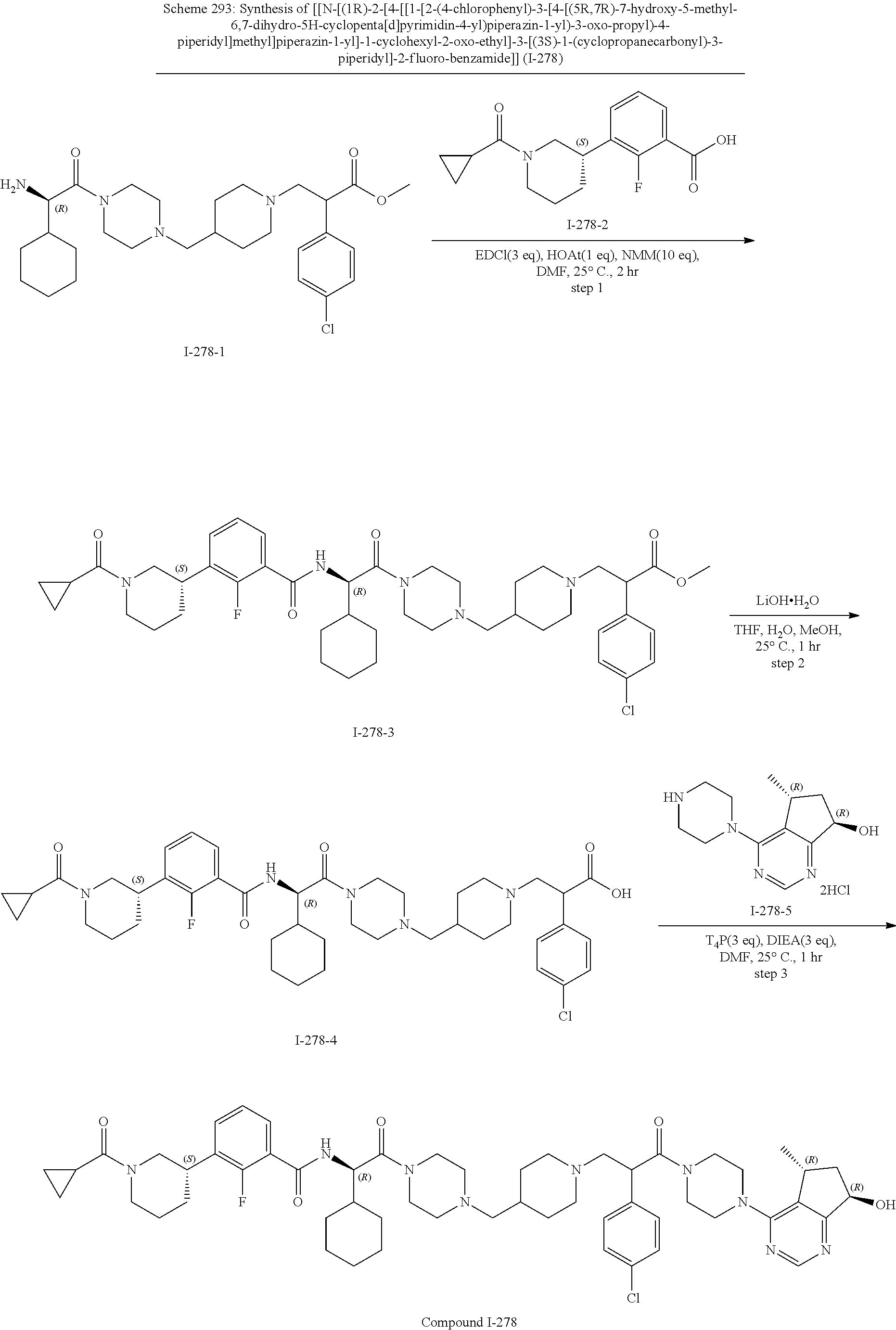

### Step 1: Synthesis of I-278-3

To a solution of I-278-1 (100 mg, 179.99 μmol, 1 eq, HCl), I-278-2 (78.65 mg, 269.99 μmol, 1.5 eq) in DMF (1 mL) was added EDCI (103.51 mg, 539.98 μmol, 3 eq) and HOAt (24.50 mg, 179.99 μmol, 25.18 μL, 1 eq), NMM (182.06 mg, 1.80 mmol, 197.89 μL, 10 eq). The mixture was stirred at 25° C. for 2 hr. The reaction mixture was quenched by addition water 5 mL at 25° C., and extracted with EA 15 mL (5 mL*3). The combined organic layers were washed with NaCl 15 mL (5 mL*3), dried over Na₂SO₄, filtered and concentrated under reduced pressure to give a residue. The residue was purified by reversed phase (26.8*125 mm, 40 g of XB—C18, 20-40 μm, 120 Å) Mobile phase: A for H₂O (0.1% NH₃·H₂O v/v) and B for acetonitrile; Gradient: B 30%-50% in 30 min; Flow rate: 60 ml/min; Column temperature: R.T. Wavelength: 220 nm/254 nm), after purification, then concentrated to remove organic solvents and then lyophilization. I-278-3 (100 mg, 112.53 μmol, 62.52% yield, 89.17% purity) was obtained as a white solid. LCMS: R^{t}=0.857 min, [M+H]⁺=792.4

### Step 2: Synthesis of I-278-4

To a solution of I-278-3 (100 mg, 126.20 μmol, 1 eq) in H₂O (1 mL) was added LiOH·H₂O (10.59 mg, 252.39 μmol, 2 eq), MeOH (1 mL), THF (1 mL). The mixture was stirred at 25° C. for 1 hr. The reaction mixture was concentrated under reduced pressure to give a residue. The residue was purified by reversed phase (26.8*125 mm, 40 g of XB—C18, 20-40 μm, 120 A) Mobile phase: A for H₂O (0.1% NH₃·H₂O v/v) and B for acetonitrile; Gradient: B 30%-50% in 30 min; Flow rate: 60 ml/min; Column temperature: R.T. Wavelength: 220 nm/254 nm). After purification, the eluent was concentrated to remove organic solvents and then lyophilized to afford the product. I-278-4 (70 mg, 87.09 μmol, 69.01% yield, 96.84% purity) was obtained as a white solid. LCMS: R^{t}=0.491 min, [M+H]⁺=778.4. SFC: R^{t}=1.783 min, 2.771 min.

### Step 3: Synthesis of I-278

To a solution of I-278-4 (60 mg, 77.08 μmol, 1 eq), I-278-5 (23.68 mg, 77.08 μmol, 1 eq, 2HCl) in DMF (0.5 mL) was added T₄P (83.31 mg, 231.25 μmol, 3 eq) and DIEA (29.89 mg, 231.25 μmol, 40.28 μL, 3 eq). The mixture was stirred at 25° C. for 1 hr. The reaction mixture was quenched by addition water 5 mL at 25° C., and extracted with EA 15 mL (5 mL*3). The combined organic layers were washed with NaCl 15 mL (5 mL*3), dried over [Na₂SO₄], filtered and concentrated under reduced pressure to give a residue. The product was purified by prep-HPLC (column: Waters Xbridge 150*25 mm*5 um; mobile phase: [water (NH₄HCO₃)-ACN]; gradient: 50%-80% B over 10 min) and the eluent was concentrated to remove MeCN and then lyophilized to afford the product. I-278 (24 mg, 24.03 μmol, 31.18% yield, 99.60% purity) was obtained as a white solid. LCMS: R^{t}=0.822 min [M+H]⁺=994.6. SFC: R^{t}=1.586 min, 1.677 min. ¹H NMR (400 MHZ, METHANOL-d4) δ=8.42 (m, 1H), 7.59-7.47 (m, 2H), 7.37-7.31 (m, 4H), 7.30-7.21 (m, 1H), 4.97 (m, 1H), 4.92 (m, 1H), 4.63-4.52 (m, 1H), 4.40 (m, 1H), 4.27 (m, 1H), 3.81 (m, 3H), 3.76-3.62 (m, 6H), 3.62-3.49 (m, 3H), 3.26 (m, 2H), 3.14-2.90 (m, 3H), 2.87-2.68 (m, 1H), 2.57-2.33 (m, 5H), 2.27-1.98 (m, 8H), 1.96-1.66 (m, 11H), 1.56 (m, 2H), 1.34-1.08 (m, 10H), 0.93-0.73 (m, 4H). ¹⁹F NMR (377 MHz, METHANOL-d4) δ=−121.66 (m, 1F).

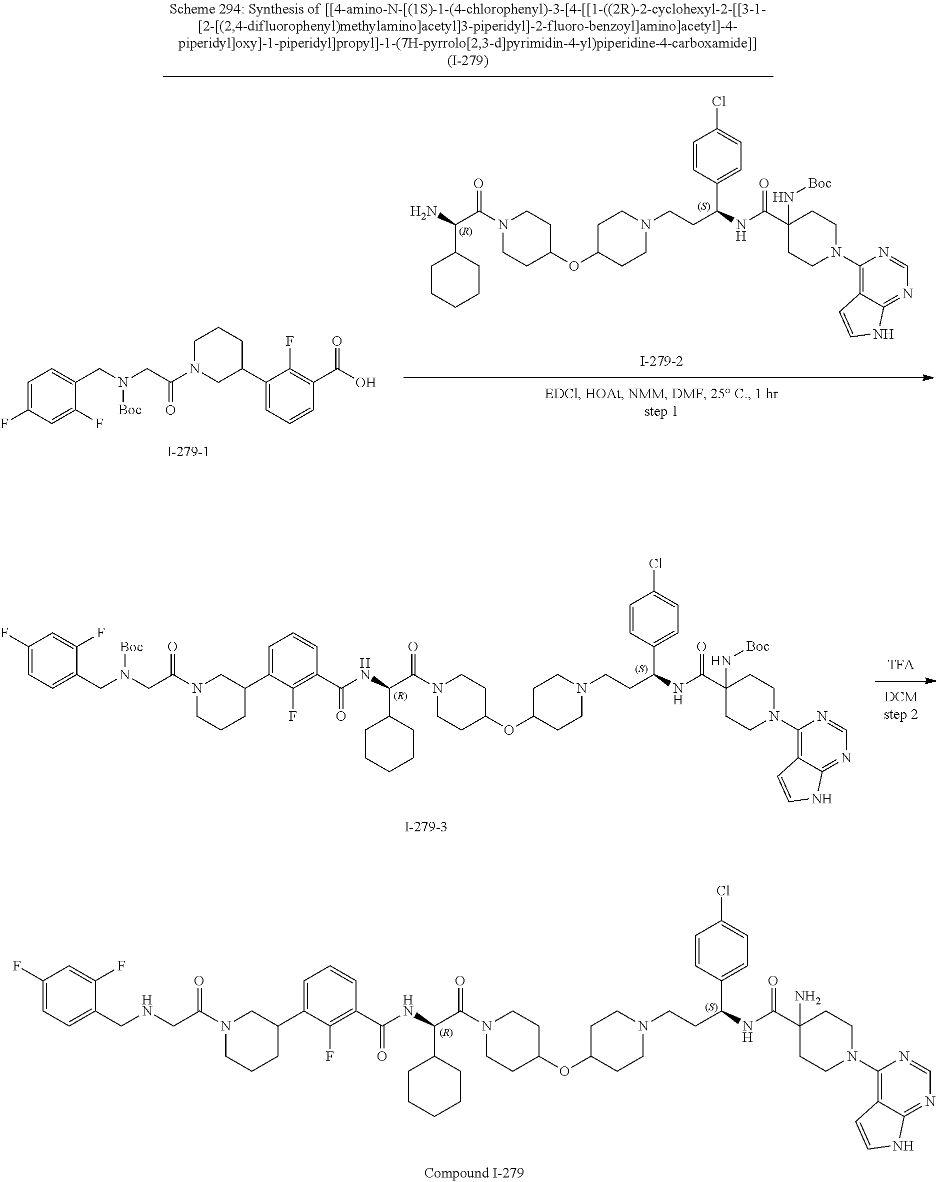

### Step 1: Synthesis of I-275-3

To a solution of I-275-1 (182.09 mg, 359.50 μmol, 1 eq) in DMF (3 mL) was added EDCI (206.75 mg, 1.08 mmol, 3 eq), NMM (181.81 mg, 1.80 mmol, 197.62 μL, 5 eq), I-275-2 (300 mg, 359.50 μmol, 1 eq) and HOAt (48.93 mg, 359.50 μmol, 50.29 UL, 1 eq). The mixture was stirred at 25° C. for 0.25 h. The reaction mixture was diluted with H₂O (10 mL) and extracted with EA 30 ml (10 ml*3). The combined organic phase was dried with anhydrous sodium sulfate, filtered and filtrate was concentrated to give crude product. The crude product was purified by prep-HPLC (column: CD02-Waters Xbidge BEH C18 150*25*10 um; mobile phase: [water (NH₄HCO₃)-ACN]; gradient: 60%-90% B over 10 min) and the eluent was lyophilized to give the product. I-275-3 (280 mg, 211.64 μmol, 58.87% yield, 100% purity) was obtained as a white solid. SFC: R^{t}=2.931 min, 3.435 min. LCMS: R^{t}=0.427 min, [M+H]⁺=1323.7

### Step 2: Synthesis of I-275

To a solution of I-275-3 (260 mg, 196.53 μmol, 1 eq) in DCM (2.5 mL) was added TFA (767.50 mg, 6.73 mmol, 0.5 mL). The mixture was stirred at 25° C. for 0.5 h. The reaction mixture was concentrated under reduced pressure to give a residue. The crude product was purified by reversed phase column (0.1% TFA condition) and the eluent was lyophilized to give the product. I-275 (222.63 mg, 176.64 μmol, 89.88% yield, 98.13% purity, TFA) was obtained as a white solid. SFC: R^{t}=2.496 min, 3.015 min. LCMS: R^{t}=0.312 min, 0.319 min, [M+H]⁺=1122.6. ¹H NMR (400 MHZ, METHANOL-d4) δ=8.35 (d, J=1.6 Hz, 1H), 7.67-7.48 (m, 3H), 7.41-7.32 (m, 5H), 7.29-7.26 (m, 1H), 7.18-7.06 (m, 2H), 6.88 (t, J=3.2 Hz, 1H), 5.04-4.94 (m, 2H), 4.62-4.57 (m, 3H), 4.33 (d, J=5.2 Hz, 2H), 4.26-4.12 (m, 2H), 4.06-3.96 (m, 1H), 3.95-3.79 (m, 4H), 3.76 (d, J=12.4 Hz, 2H), 3.65-3.46 (m, 2H), 3.37 (s, 1H), 3.29-3.14 (m, 4H), 3.12-3.01 (m, 2H), 2.91-2.74 (m, 1H), 2.71-2.54 (m, 2H), 2.42-2.30 (m, 1H), 2.29-2.10 (m, 3H), 2.01 (d, J=13.2 Hz, 4H), 1.98-1.88 (m, 4H), 1.88-1.73 (m, 6H), 1.73-1.63 (m, 4H), 1.59-1.49 (m, 1H), 1.35-1.20 (m, 3H), 1.19-1.09 (m, 2H). ¹⁹F NMR (377 MHz, METHANOL-d4) δ=−76.96 (s, 10F), —108.18-−108.47 (m, 1F), −112.67-−112.98 (m, 1F), −121.50-−122.02 (m, 1F).

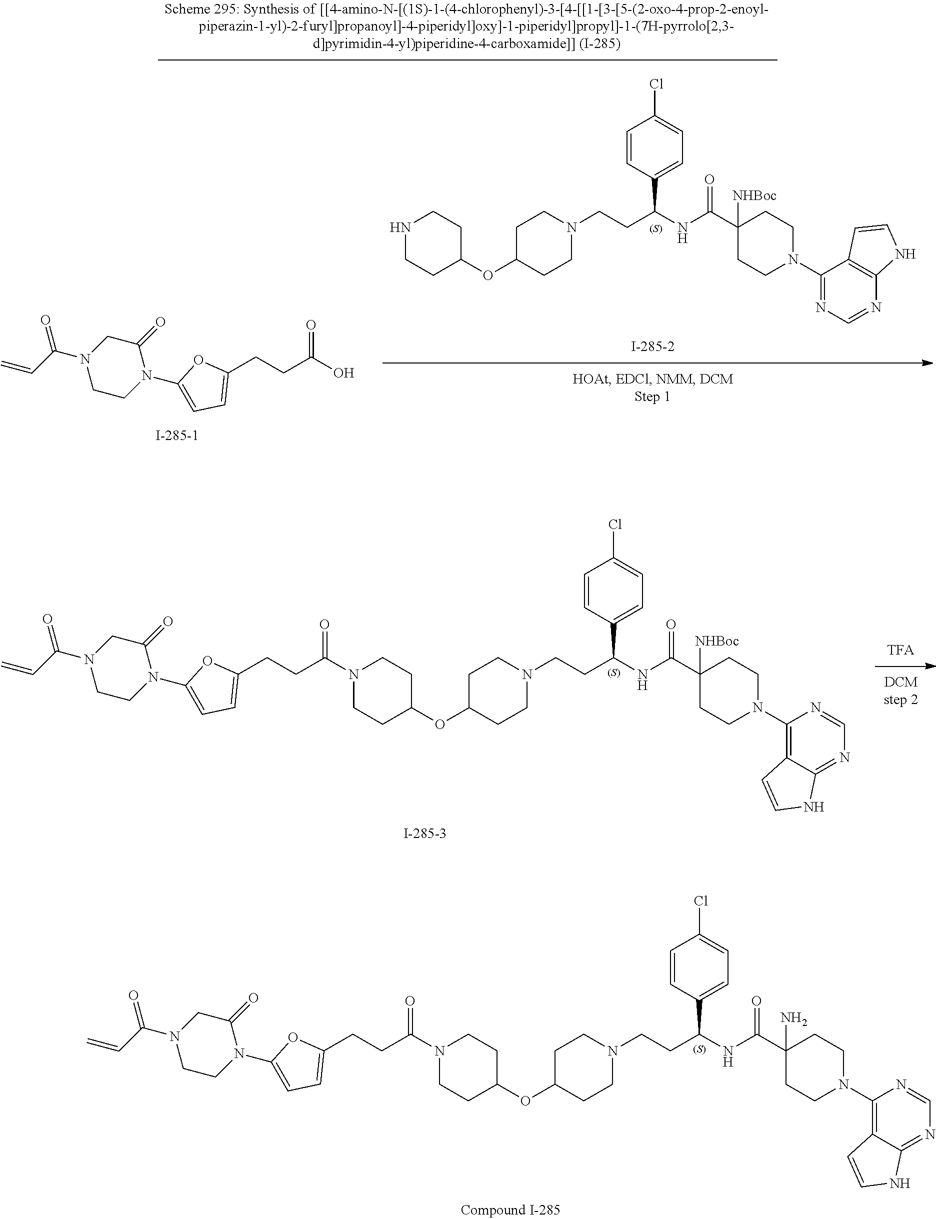

### Step 1: Synthesis of I-285-3

To a solution of I-285-2 (118.94 mg, 171.06 μmol, 1 eq) in DMF (2 mL) was added EDCI (98.38 mg, 513.19 μmol, 3 eq), HOAt (23.28 mg, 171.06 μmol, 23.93 μL, 1 eq), I-285-1 (50 mg, 171.06 μmol, 1 eq) and NMM (86.51 mg, 855.32 μmol, 94.04 μL, 5 eq). The mixture was stirred at 25° C. for 1 hr. The mixture was diluted with H₂O (5 mL) and extracted with EA 10 mL (5 mL*2). The combined organic layers were washed with brine (10 mL), dried over Na₂SO₄, filtered and concentrated under reduced pressure to give a residue. The residue was purified by flash silica gel chromatography (ISCO®; 12 g SepaFlash® Silica Flash Column, Eluent of 0˜20% MeOH/DCM gradient @ 60 mL/min). The eluent was concentrated under reduced pressure to give I-285-3 (80 mg, 78.06 μmol, 45.63% yield, 94.603% purity) as a yellow solid. LCMS: R^{t}=0.334 min, [M+H]⁺=969.5

### Step 2: Synthesis of I-285

To a solution of I-285-3 (80 mg, 82.51 μmol, 1 eq) in DCM (0.8 mL) and TFA (0.16 mL) was stirred at 25° C. for 1.5 hr. The mixture was concentrated in vacuum. The filtrate was purified by reversed-phase column (0.1% TFA condition). The eluent was concentrated under reduced pressure to remove ACN and then lyophilized to give I-285 (25.56 mg, 25.99 μmol, 31.50% yield, 100% purity, TFA) as a yellow solid. LCMS: R^{t}=0.275 min, [M+H]⁺=869.4. ¹H NMR (400 MHZ, METHANOL-d4) δ=8.39 (s, 1H), 7.42-7.30 (m, 5H), 6.94 (d, J=3.6 Hz, 1H), 6.86-6.67 (m, 1H), 6.34-6.25 (m, 1H), 6.23-6.10 (m, 1H), 5.88-5.78 (m, 1H), 5.04-4.97 (m, 2H), 4.67-4.57 (m, 2H), 4.51-4.25 (m, 2H), 4.06-3.64 (m, 10H), 3.64-3.48 (m, 1H), 3.37 (s, 2H), 3.29-3.15 (m, 3H), 3.15-2.95 (m, 2H), 2.95-2.57 (m, 6H), 2.41-2.13 (m, 4H), 2.09-1.86 (m, 4H), 1.85-1.67 (m, 2H), 1.62-1.40 (m, 2H). SFC: R^{t}=1.134 min. ¹⁹F NMR (377 MHz, METHANOL-d4) δ=−77.089.

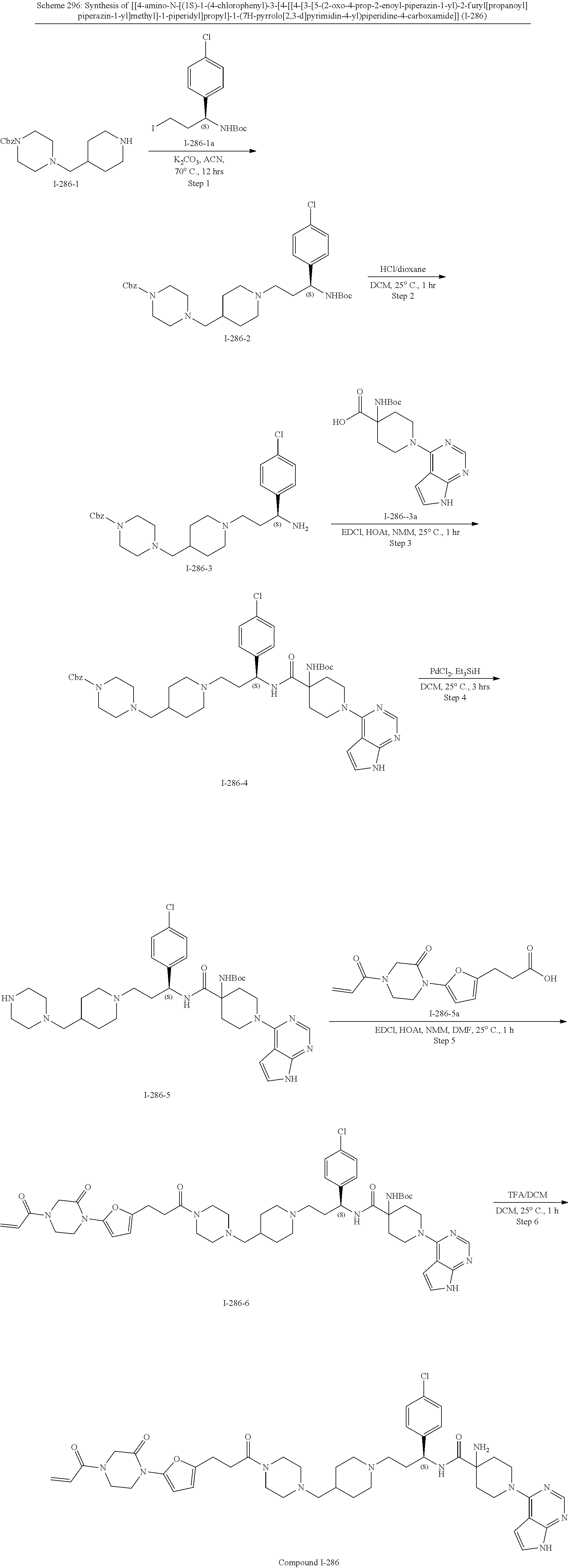

### Step 1: Synthesis of I-286-2

To a solution of I-286-1a (500 mg, 1.26 mmol, 1 eq) in ACN (5 mL) was added I-286-1 (401.13 mg, 1.26 mmol, 1 eq) and K₂CO₃ (523.95 mg, 3.79 mmol, 3 eq). The mixture was stirred at 70° C. for 12 hrs. The reaction mixture was diluted with H₂O 10 mL and extracted with EA 10 mL (5 mL*2). The combined organic layers were dried over Na₂SO₄, filtered and concentrated under reduced pressure to give a residue. The residue was purified by flash silica gel chromatography (ISCO®; 20 g SepaFlash® Silica Flash Column, Eluent of 0˜25% MeOH/EA gradient (60 mL/min). The eluent was concentrated under reduced pressure to give I-286-2 (670 mg, 1.13 mmol, 89.13% yield, 98.37% purity) was obtained as a yellow solid. LCMS: R^{t}=0.321 min, [M+H]⁺=585.4. SFC: R^{t}=1.560 min. ¹H NMR (400 MHZ, METHANOL-d4) δ=7.37-7.26 (m, 9H), 5.11 (s, 2H), 4.61-4.51 (m, 1H), 3.52-3.45 (m, 4H), 3.05-2.92 (m, 2H), 2.54-2.45 (m, 1H), 2.42-2.35 (m, 5H), 2.23-2.17 (m, 2H), 2.15-2.04 (m, 2H), 1.95-1.87 (m, 2H), 1.85-1.75 (m, 2H), 1.67-1.53 (m, 1H), 1.41 (s, 9H), 1.34-1.26 (m, 2H).

### Step 2: Synthesis of I-286-3

To a solution of I-286-2 (500 mg, 854.44 μmol, 1 eq) in DCM (2 mL) and HCl/dioxane (2 M, 5 mL) was stirred at 25° C. for 1 hr. The mixture was concentrated in vacuum to give I-286-3 (450 mg, crude, HCl) as a yellow solid was used into the next step without further purification. LCMS: R^{t}=0.220 min, [M+H]⁺=485.2

### Step 3: Synthesis of I-286-4

To a solution of I-286-3 (450 mg, 862.86 μmol, 1 eq, HCl) in DMF (5 mL) was added I-286-3a (311.83 mg, 862.86 μmol, 1 eq), EDCI (496.23 mg, 2.59 mmol, 3 eq), HOAt (117.44 mg, 862.86 μmol, 120.70 μL, 1 eq) and NMM (436.38 mg, 4.31 mmol, 474.33 μL, 5 eq). The mixture was stirred at 25° C. for 1 hr. The reaction mixture was diluted with H₂O 5 mL and extracted with EA 10 mL (5 mL*2). The combined organic layers were washed with brine 2 mL (2 mL*1), dried over Na₂SO₄, filtered and concentrated under reduced pressure to give a residue. The residue was purified by flash silica gel chromatography (ISCO®; 80 g SepaFlash® Silica Flash Column, Eluent of 0˜30% MeOH/EA gradient @ 60 mL/min). The eluent was concentrated under reduced pressure to give I-286-4 (420 mg, 506.98 μmol, 58.76% yield, 100% purity) was obtained as a yellow solid. LCMS: R^{t}=0.302 min, [M+H]⁺=828.5. SFC: R^{t}=1.352 min.

### Step 4: Synthesis of I-286-5

To a solution of I-286-4 (370 mg, 446.62 μmol, 1 eq) in DCM (2 mL) was added PdCl₂ (23.76 mg, 133.99 μmol, 0.3 eq) and TEA (90.39 mg, 893.25 μmol, 124.33 μL, 2 eq). The reaction mixture was degassed with N₂. Then a solution of Et₃SiH (207.73 mg, 1.79 mmol, 285.34 μL, 4 eq) in DCM (2 mL) was added dropwise at 10° C. The mixture was stirred at 25° C. for 3 hrs. The mixture was filtered and the filtrate was concentrated in vacuum. Then the mixture diluted with NaHCO₃ 5 mL and extracted with CH₂Cl, 5 mL (2.5 mL*2). The combined organic layers were dried over Na₂SO₄, filtered and concentrated under reduced pressure to give a residue. The crude product was purified by reversed-phase column (0.1% FA condition). The eluent was concentrated under reduced pressure to remove ACN and then lyophilized to give the product. The product was dissolved with DCM 2 mL and diluted with NH₃·H₂O 2 mL 10 mL, and extracted with DCM 4 mL (2 mL×2). The combined organic layers were dried over Na₂SO₄, filtered and concentrated under reduced pressure to remove DCM and then lyophilized to give I-286-5 (150 mg, 196.60 μmol, 44.02% yield, 91% purity) was obtained as a yellow solid. LCMS: R^{t}=0.238 min, [M+H]⁺=694.4

### Step 5: Synthesis of I-286-6

To a solution of I-286-5 (80 mg, 115.22 μmol, 1 eq) in DMF (1 mL) was added I-286-5a (33.68 mg, 115.22 μmol, 1 eq), EDCI (66.26 mg, 345.67 μmol, 3 eq), HOAt (15.68 mg, 115.22 μmol, 16.12 μL, 1 eq) and NMM (58.27 mg, 576.11 μmol, 63.34 μL, 5 eq). The mixture was stirred at 25° C. for 1 hr. The reaction mixture was diluted with H₂O 2 mL and extracted with EA 6 mL (3 mL*2). The combined organic layers were washed with brine 2 mL (2 mL*1), dried over Na₂SO₄, filtered and concentrated under reduced pressure to give a residue. The crude product was purified by reversed-phase column (0.1% NH₃·H₂O condition). The eluent was concentrated under reduced pressure to remove ACN and then lyophilized to give I-286-6 (90 mg, 71.55 μmol, 62.10% yield, 77% purity) was obtained as a yellow solid. LCMS: R^{t}=0.294 min, [M+H]⁺=968.8

### Step 6: Synthesis of I-286

To a solution of I-286-6 (80 mg, 82.60 μmol, 1 eq) in DCM (1 mL) and TFA (0.2 mL) was stirred at 25° C. for 1 hr. The reaction mixture was diluted with NaHCO₃ 3 mL to adjust pH=8 and extracted with DCM 6 mL (3 mL*2). The combined organic layers were dried over Na₂SO₄, filtered and concentrated under reduced pressure to give a residue. The crude product was purified by reversed-phase column (0.1% NH₃·H₂O condition). The eluent was concentrated under reduced pressure to remove ACN and then lyophilized to give I-286 (17.85 mg, 20.55 μmol, 24.88% yield, 100% purity) was obtained as a white solid. LCMS: R^{t}=0.245 min, [M+H]⁺=868.4. SFC: R^{t}=1.005 min. ¹H NMR (400 MHZ, METHANOL-d4) δ=8.54 (s, 1H), 8.13 (s, 1H), 7.33 (s, 3H), 7.12 (d, J=3.6 Hz, 1H), 6.87-6.66 (m, 1H), 6.62 (d, J=3.6 Hz, 1H), 6.41-6.18 (m, 2H), 6.11 (d, J=3.2 Hz, 1H), 5.91-5.73 (m, 1H), 4.92-4.87 (m, 2H), 4.59-4.34 (m, 4H), 4.12-3.74 (m, 4H), 3.70-3.60 (m, 2H), 3.60-3.42 (m, 4H), 3.14-2.84 (m, 4H), 2.78-2.62 (m, 2H), 2.36-2.29 (m, 4H), 2.52-2.27 (m, 2H), 2.25-1.97 (m, 8H), 1.84-1.73 (m, 2H), 1.66-1.50 (m, 3H), 1.33-1.20 (m, 2H). ¹⁹F NMR (377 MHz, MeOD-d4) δ=−76.909.

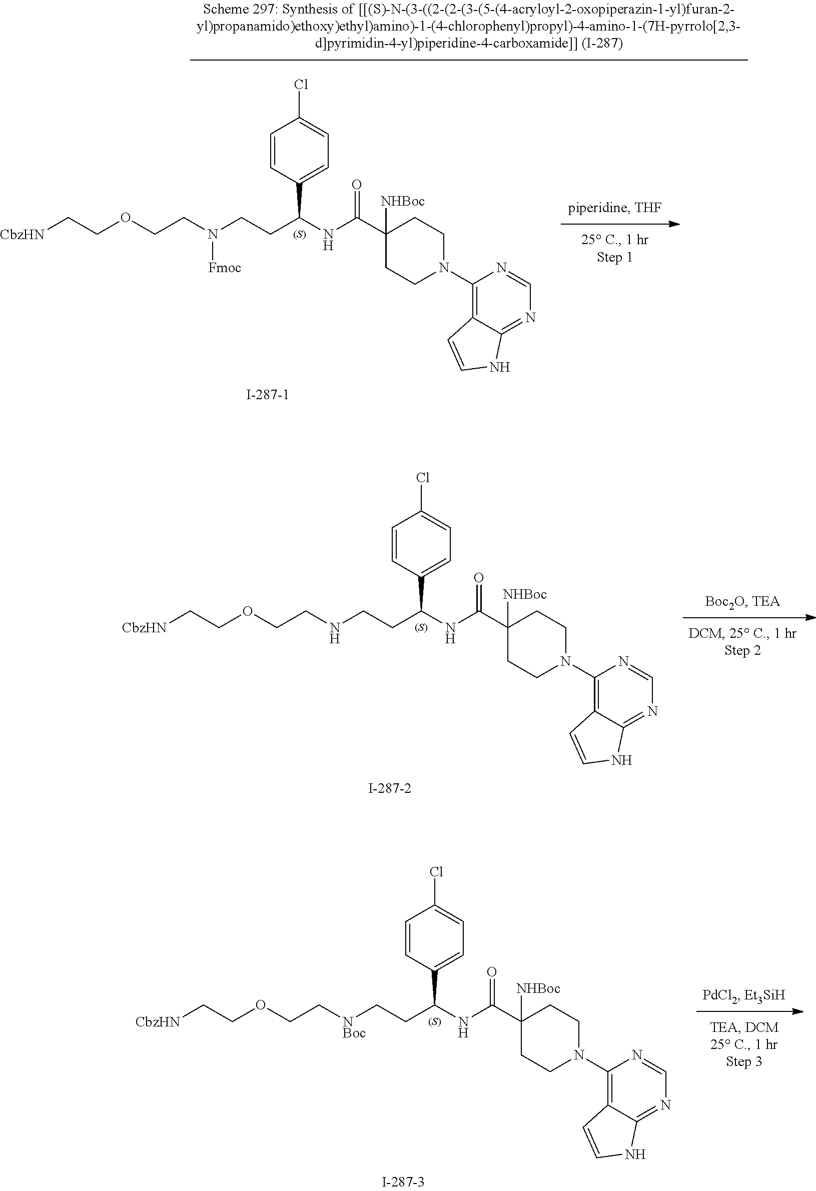

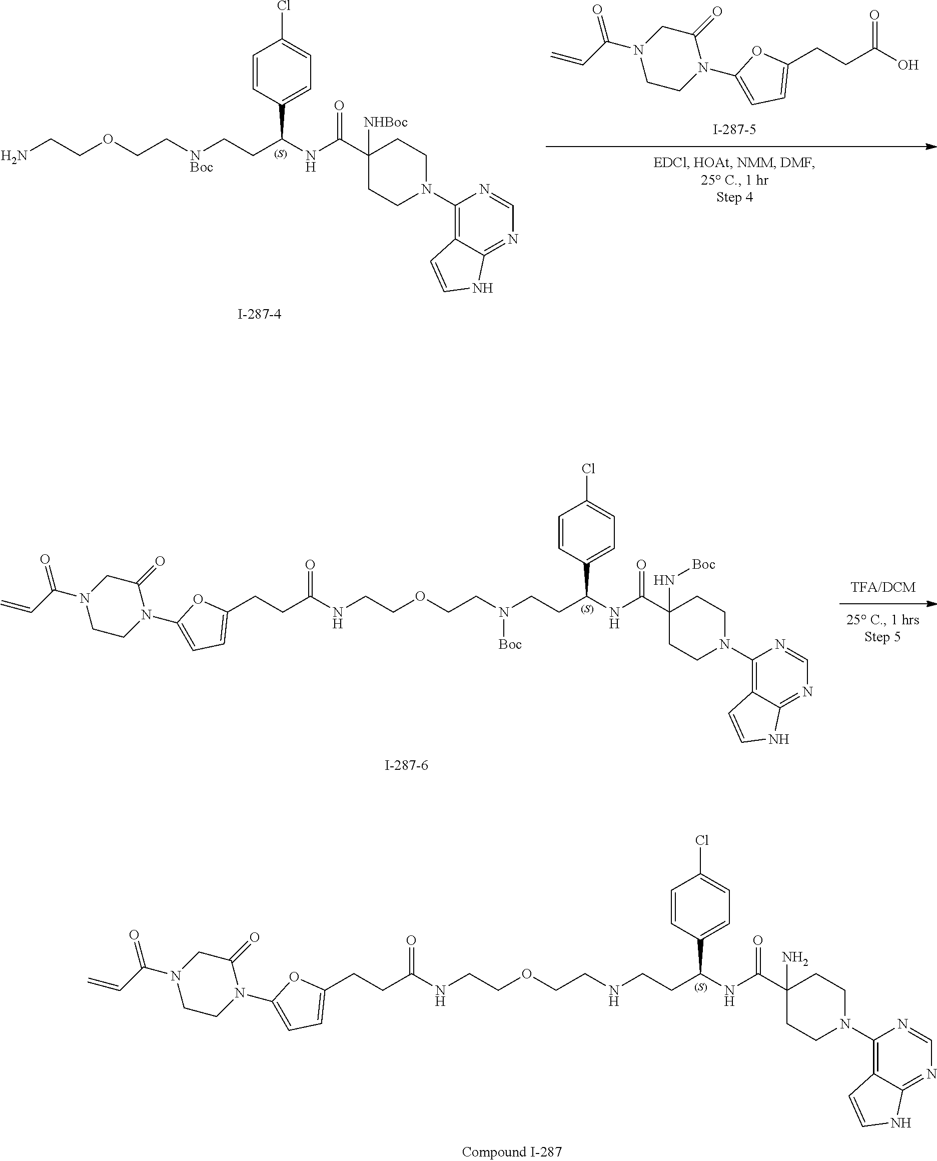

### Step 1: Synthesis of I-287-2.

To a solution of I-287-1 (1 g, 1.03 mmol, 1 eq) in THF (8 mL) was added PIPERIDINE (1 mL). The mixture was stirred at 25° C. for 1 hr. The reaction mixture was concentrated under reduced pressure to give a residue. The crude product was purified by reversed-phase column (0.1% NH₃·H₂O), the eluent was concentrated and lyophilized to give the desired product. I-287-2 (700 mg, 920.78 μmol, 89.46% yield, 98.563% purity) was obtained as a pale yellow solid. LCMS: R^{t}=0.341 min, [M+H]⁺=749.4. SFC: R^{t}=1.739 min. ¹H NMR (400 MHZ, METHANOL-d4) δ=8.12 (s, 1H), 7.34-7.24 (m, 9H), 7.11 (d, J=3.6 Hz, 1H), 6.59 (d, J=3.6 Hz, 1H), 5.05-4.98 (m, 3H), 4.46-4.34 (m, 2H), 3.64-3.40 (m, 7H), 3.30-3.18 (m, 2H), 2.76-2.61 (m, 4H), 2.18-2.06 (m, 4H), 2.03-1.89 (m, 2H), 1.43 (s, 9H).

### Step 2: Synthesis of I-287-3.

To a solution of I-287-2 (550 mg, 734.02 μmol, 1 eq) in DCM (6 mL) was added TEA (222.82 mg, 2.20 mmol, 306.50 μL, 3 eq) and Boc₂O (176.22 mg, 807.42 μmol, 185.49 μL, 1.1 eq). The mixture was stirred at 25° C. for 1 hr. The reaction mixture was concentrated under reduced pressure to give a residue. The residue was purified by flash silica gel chromatography (ISCO®; 20 g SepaFlash® Silica Flash Column, Eluent of 0˜100% Ethyl acetate/Petroleum ether gradient (@, 60 mL/min), the eluent was concentrated under reduced pressure to give a residue. I-287-3 (600 mg, 706.37 μmol, 96.23% yield, 100% purity) was obtained as a white solid. LCMS: R^{t}=0.442 min, [M+H]′=849.5. SFC: R^{t}=1.171 min. 1H NMR (400 MHZ, CHLOROFORM-d) δ=10.32-10.07 (m, 1H), 8.32 (s, 1H), 7.37-7.28 (m, 5H), 7.26-7.19 (m, 4H), 7.07 (d, J=3.6 Hz, 1H), 6.50 (d, J=2.4 Hz, 1H), 5.57-5.28 (m, 1H), 5.18-4.99 (m, 3H), 4.91 (s, 1H), 4.45-4.26 (m, 2H), 3.71-3.57 (m, 2H), 3.54-3.16 (m, 11H), 2.37-2.22 (m, 2H), 2.18-2.07 (m, 2H), 1.96 (s, 2H), 1.42 (s, 18H).

### Step 3: Synthesis of I-287-4.

To a solution of I-287-3 (250 mg, 294.32 μmol, 1 eq) in DCM (2 mL) was added PdCl₂ (5.22 mg, 29.43 μmol, 0.1 eq) and TEA (44.67 mg, 441.48 μmol, 61.45 μL, 1.5 eq) degassed and purged with N₂ for 3 times. Then Et₃SiH (102.67 mg, 882.96 μmol, 141.03 μL, 3 eq) in DCM (1 mL) was added into the system at 0° C. The mixture was stirred at 25° C. for 1 hr under N₂ atmosphere. The mixture was adjusted to pH=8 with NaHCO₃ and diluted with H₂O 5 mL and extracted with EA 15 mL (5 mL*3). The combined organic layers were dried over Na₂SO₄, filtered and concentrated under reduced pressure to give a residue. The crude product was purified by reversed-phase column (neutral condition), the eluent was concentrated and lyophilized to give the desired product. I-287-4 (150 mg, 199.82 μmol, 67.89% yield, 95.283% purity) was obtained as a white solid. LCMS: R^{t}=0.341 min, [M+H]′=715.3. SFC: R^{t}=1.492 min. ¹H NMR (400 MHz, METHANOL-d4) δ=8.12 (s, 1H), 7.32 (s, 4H), 7.12 (d, J=3.6 Hz, 1H), 6.62 (d, J=3.6 Hz, 1H), 4.47-4.31 (m, 2H), 3.62-3.37 (m, 9H), 2.79-2.72 (m, 2H), 2.19-2.04 (m, 6H), 1.47-1.40 (m, 18H), 1.29 (s, 2H).

### Step 4: Synthesis of I-287-6.

To a solution of I-287-5 (53.12 mg, 181.75 μmol, 1 eq) in DMF (1 mL) was added HOAt (24.74 mg, 181.75 μmol, 25.42 μL, 1 eq), EDCI (84.64 mg, 545.24 μmol, 96.51 μL, 3 eq) and NMM (91.92 mg, 908.73 μmol, 99.91 μL, 5 eq). Then I-287-4 (130 mg, 181.75 μmol, 1 eq) was added into the system. The mixture was stirred at 25° C. for 0.5 hr. The reaction mixture was diluted with H₂O 5 mL and extracted with EA 15 mL (5 mL*3). The combined organic layers were washed with brine 15 mL (5 mL*3), dried over Na₂SO₄, filtered and concentrated under reduced pressure to give a residue. The crude product was purified by reversed-phase column (0.1% FA condition), the eluent was concentrated and lyophilized to give the desired product. I-287-6 (80 mg, 77.25 μmol, 42.51% yield, FA) was obtained as a yellow solid.

LCMS: R^{t}=0.414 min, [M+H]⁺=989.4. ¹H NMR (400 MHZ, METHANOL-d4) δ=8.45 (s, 1H), 8.21-8.03 (m, 2H), 7.36-7.24 (m, 4H), 7.12 (d, J=3.6 Hz, 1H), 6.61 (d, J=3.6 Hz, 1H), 6.32-6.24 (m, 1H), 6.21 (d, J=3.2 Hz, 1H), 6.07 (d, J=3.2 Hz, 1H), 5.84-5.78 (m, 1H), 4.89 (s, 1H), 4.47-4.33 (m, 4H), 3.96 (d, J=1.6 Hz, 2H), 3.88-3.78 (m, 2H), 3.64-3.42 (m, 7H), 3.39-3.32 (m, 3H), 3.26-3.12 (m, 2H), 2.91-2.83 (m, 2H), 2.52-2.43 (m, 2H), 2.20-2.03 (m, 6H), 1.43 (s, 18H).

### Step 5: Synthesis of I-287.

To a solution of I-287-6 (50 mg, 48.28 μmol, 1 eq, FA) in DCM (0.5 mL) was added TFA (0.1 mL). The mixture was stirred at 25° C. for 0.5 hr. The reaction mixture was concentrated under reduced pressure to give a residue. The residue was purified by prep-HPLC (column: CD06-Waters Xbidge C18 150*40*10 um; mobile phase: [water (TFA)-ACN]; gradient: 4%-34% B over 10 min), the eluent was concentrated and lyophilized to give the desired product. I-287 (21.09 mg, 23.35 μmol, 48.35% yield, 100% purity; TFA) was obtained as a white solid. LCMS: R^{t}=0.261 min, [M+H]⁺=789.5. SFC: R^{t}=0.597 min. ¹H NMR (400 MHZ, METHANOL-d4) δ=8.35 (s, 1H), 7.35 (s, 4H), 7.34 (d, J=3.6 Hz, 1H), 6.88 (d, J=3.6 Hz, 1H), 6.83-6.66 (m, 1H), 6.30 (d, J=16.6 Hz, 1H), 6.21 (s, 1H), 6.08 (d, J=3.2 Hz, 1H), 5.87-5.78 (m, 1H), 5.08-4.99 (m, 1H), 4.71-4.60 (m, 2H), 4.46-4.31 (m, 2H), 4.00 (d, J=4.0 Hz, 2H), 3.86-3.74 (m, 4H), 3.66 (s, 2H), 3.54-3.48 (m, 2H), 3.41-3.34 (m, 1H), 3.23-3.05 (m, 4H), 2.96 (d, J=4.0 Hz, 1H), 2.9 I-287-2.85 (m, 2H), 2.69-2.56 (m, 2H), 2.54-2.47 (m, 2H), 2.33-2.19 (m, 2H), 2.15 (d, J=14.8 Hz, 1H), 2.02 (d, J=14.4 Hz, 1H). ¹⁹F NMR (376 MHz, METHANOL-d4) δ=−77.067.

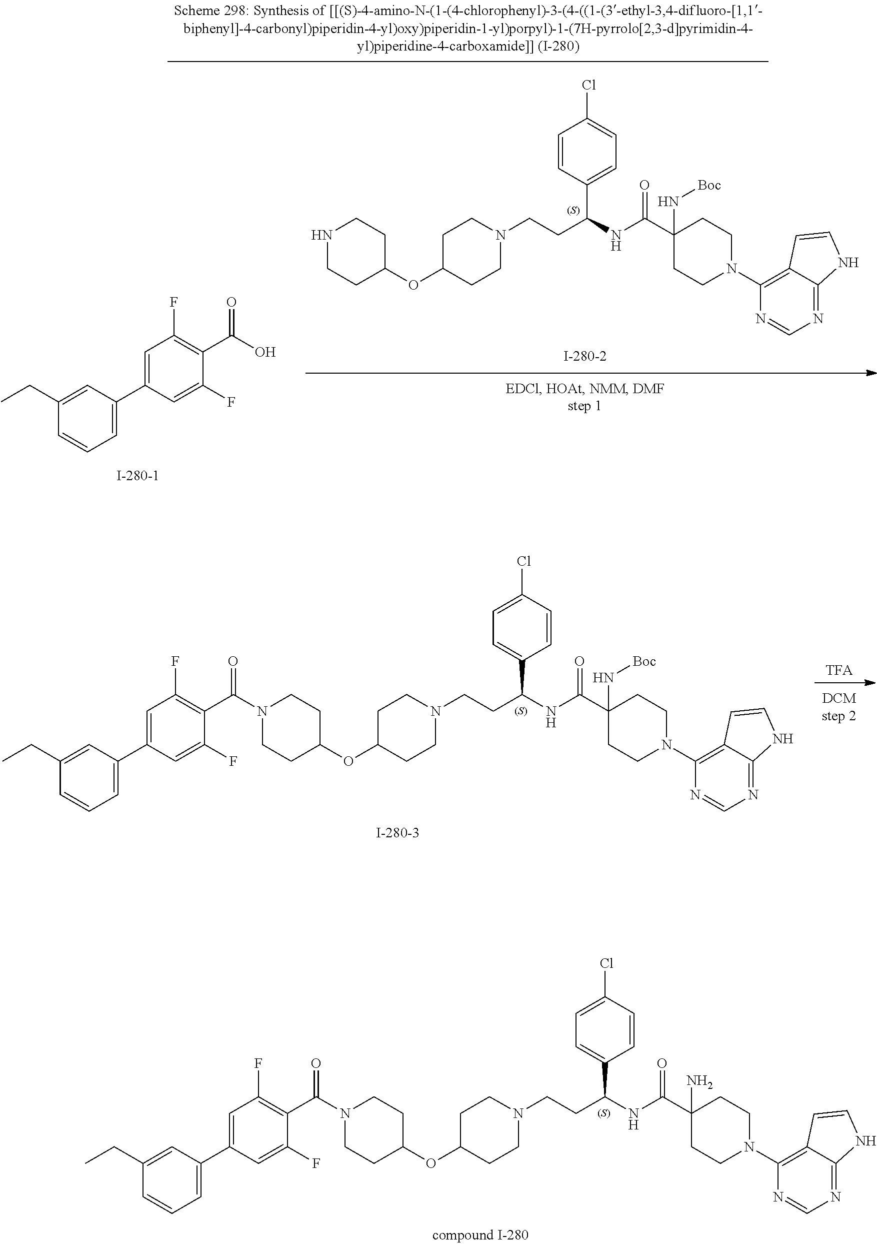

### Step 1: Synthesis of I-280-3

To a solution of I-280-1 (226.31 mg, 862.95 μmol, 1.2 eq), I-280-2 (0.5 g, 719.12 μmol, 1 eq) in DMF (5 mL) was added HOAt (97.88 mg, 719.12 μmol, 100.60 μL, 1 eq) and NMM (363.69 mg, 3.60 mmol, 395.31 μL, 5 eq), EDCI (413.57 mg, 2.16 mmol, 3 eq). The mixture was stirred at 25° C. for 0.5 hr. The reaction mixture was diluted with H₂O 5 mL and extracted with DCM 20 mL (10 mL*2). The combined organic layers were washed with NaCl (aq) 20 mL (10 mL*2), dried over Na₂SO₄, filtered and concentrated under reduced pressure to give a residue. The residue was purified by prep-HPLC (column: Phenomenex luna C18 150*40 mm*15 um; mobile phase: [water (FA)-ACN]; gradient: 21%-51% B over 10 min) the eluent was concentrated to remove ACN and lyophilized to get product. I-280-3 (0.35 g, 347.61 umol, 48.34% yield, 93.31% purity) was obtained as a yellow solid. LCMS: R^{t}=0.426 min, [M+H]⁺=939.4. SFC: R^{t}=1.541 min.

### Step 2: Synthesis of 1-280

To a solution of I-280-3 (0.33 g, 351.24 μmol, 1 eq) in DCM (4 mL) was added TFA (1.23 g, 10.77 mmol, 0.8 mL, 30.66 eq). The mixture was stirred at 25° C. for 0.5 hr. The reaction mixture was concentrated under reduced pressure to give a residue. The residue was purified by reversed phase (0.1% NH₃·H₂O) the eluent was concentrated to remove ACN and lyophilized to get product. I-280 (128.87 mg, 149.01 μmol, 42.42% yield, 97.06% purity) was obtained as a yellow solid. LCMS: R^{t}=0.418 min, [M+H]⁺=839.5. SFC: R^{t}=0.820 min. ¹H NMR (400 MHZ, METHANOL-d4) δ=8.12 (s, 1H), 7.52-7.45 (m, 2H), 7.42-7.35 (m, 3H), 7.35-7.26 (m, 5H), 7.12 (d, J=3.6 Hz, 1H), 6.62 (d, J=3.6 Hz, 1H), 4.92-4.89 (m, 1H), 4.54-4.43 (m, 2H), 4.11-4.00 (m, 1H), 3.79-3.76 (m, 1H), 3.70-3.55 (m, 4H), 3.53-3.46 (m, 1H), 3.29-3.24 (m, 1H), 2.76-2.70 (m, 3H), 2.39-2.31 (m, 2H), 2.28-2.10 (m, 4H), 2.08-1.92 (m, 3H), 1.92-1.79 (m, 4H), 1.63 (s, 3H), 1.61-1.52 (m, 3H), 1.30-1.26 (m, 3H). ¹⁹F NMR (376 MHz, METHANOL-d4) δ=−115.42 (br d, J=45.2 Hz, 2F).

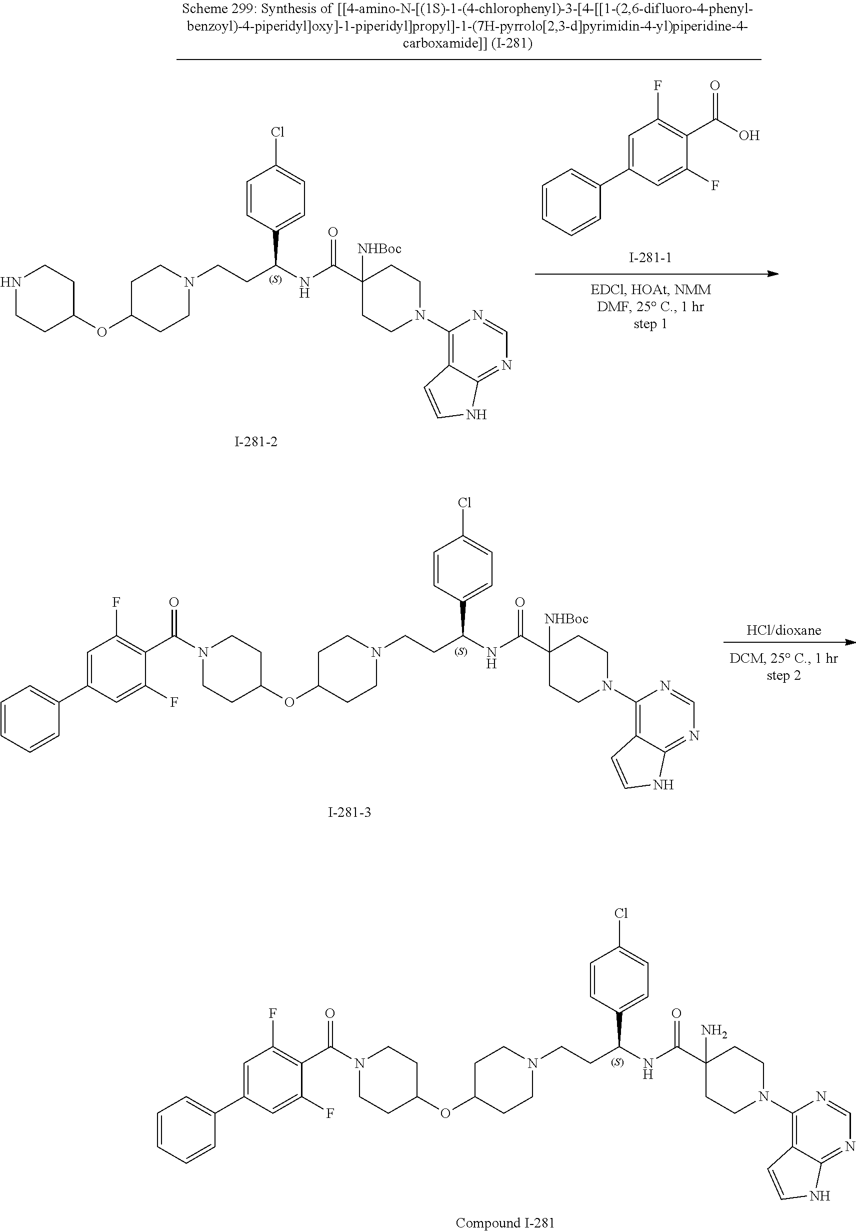

### Step 1: Synthesis of I-281-3

To a solution of I-281-1 (150 mg, 640.48 μmol, 1 eq) in DMF (5 mL) was added HOAt (87.18 mg, 640.48 μmol, 89.60 μL, 1 eq), EDCI (368.34 mg, 1.92 mmol, 3 eq), NMM (323.92 mg, 3.20 mmol, 352.08 μL, 5 eq) and I-281-2 (445.32 mg, 640.48 μmol, 1 eq). The mixture was stirred at 25° C. for 1 hr. The mixture was diluted with water (10 mL) and extracted with EA (5 mL+3). The combined organic layers dried over sodium sulfate, filtered and concentrated under reduced pressure to give a residue. The residue was purified by flash silica gel chromatography (ISCO®; 12 g SepaFlash® Silica Flash Column, Eluent of 0˜10% EA/MeOH gradient (40 mL/min) and then the eluent was concentrated to give product. I-281-3 (320 mg, 347.63 μmol, 54.28% yield, 99.017% purity) was obtained as a yellow gum. LCMS: R^{t}=0.311 min, [M+H]⁺=911.5. ¹H NMR (400 MHz, METHANOL-d4) δ=8.12 (s, 1H), 7.68-7.65 (m, 2H), 7.49-7.37 (m, 5H), 7.31 (m, 4H), 7.12 (d, J=3.6 Hz, 1H), 6.61 (d, J=3.6 Hz, 1H), 4.99-4.94 (m, 1H), 4.38 (s, 2H), 4.09-4.02 (m, 1H), 3.79 (m, 1H), 3.66-3.55 (m, 5H), 3.30-3.25 (m, 1H), 2.87 (d, J=7.6 Hz, 1H), 2.80 (d, J=2.0 Hz, 1H), 2.47 (s, 2H), 2.42-2.28 (m, 2H), 2.11 (s, 3H), 2.07 (d, J=6.8 Hz, 1H), 1.99 (d, J=7.2 Hz, 1H), 1.97-1.88 (m, 4H), 1.87-1.80 (m, 1H), 1.76-1.50 (m, 5H), 1.46 (s, 9H). ¹⁹F NMR (376 MHz, METHANOL-d4) δ=−115.20 (br s, 1F), −115.29 (s, 1F).

### Step 2: Synthesis of I-281

To a solution of I-281-3 (270 mg, 296.22 μmol, 1 eq) in DCM (2.7 mL) was added HCl/dioxane (2 M, 2.7 mL). The mixture was stirred at 25° C. for 1 hr. The mixture was adjust to pH=8 with aq. NaHCO₃ solution, and then extracted with DCM (3 mL*3). The combined organic layers dried over sodium sulfate, filtered and concentrated under reduced pressure to give a residue. The residue was purified by prep-HPLC (column: CD02-Waters Xbidge BEH C18 150*25*10 um; mobile phase: [water (NH₄HCO₃)-ACN]; gradient: 43%-73% B over 10 min), the eluent was concentrated under reduced pressure to remove ACN and lyophilized to give product. I-281 (150.68 mg, 174.46 μmol, 70.78% yield, 99.27% purity, FA) was obtained as a white solid. LCMS: R^{t}=0.729 min, [M+H]⁺=811.4. SFC: R^{t}=0.908 min. ¹H NMR (400 MHz, METHANOL-d4) δ=8.54 (s, 1H), 8.12 (s, 1H), 7.67 (d, J=7.6 Hz, 2H), 7.51-7.45 (m, 2H), 7.45-7.36 (m, 3H), 7.33 (s, 4H), 7.12 (d, J=3.6 Hz, 1H), 6.62 (d, J=3.6 Hz, 1H), 4.91-4.90 (m, 1H), 4.54-4.43 (m, 2H), 4.09-4.01 (m, 1H), 3.78-3.77 (m, 1H), 3.70-3.62 (m, 2H), 3.60-3.59 (m, 1H), 3.56-3.55 (m, 2H), 3.30-3.24 (m, 1H), 2.91-2.74 (m, 2H), 2.51-2.29 (m, 4H), 2.24-2.18 (m, 1H), 2.17-2.11 (m, 1H), 2.03-2.02 (m, 2H), 1.95-1.81 (m, 4H), 1.71-1.62 (m, 3H), 1.62-1.56 (m, 2H), 1.55 (s, 1H). ¹⁹F NMR (376 MHZ, METHANOL-d4) δ=−115.254, −115.359.

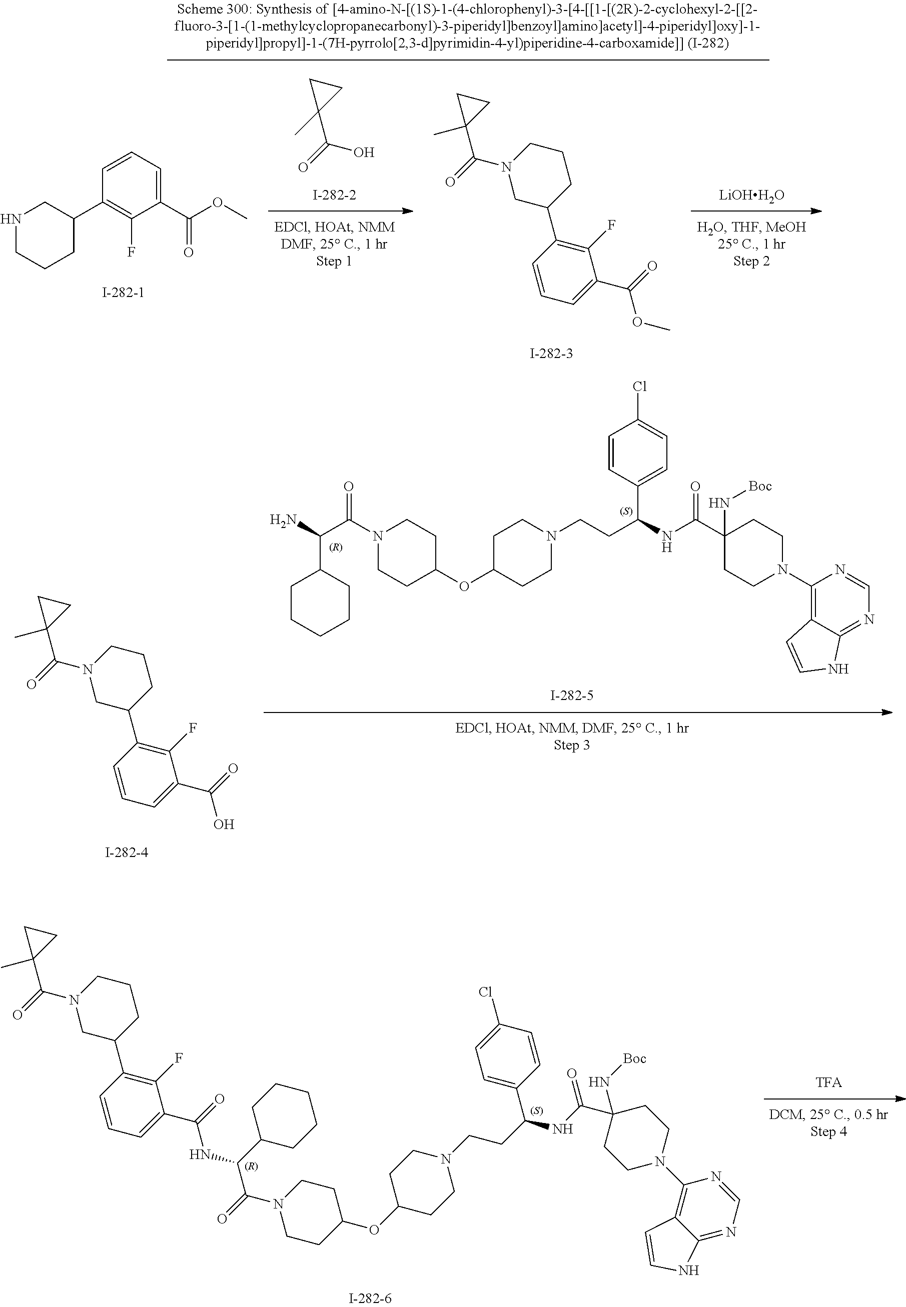

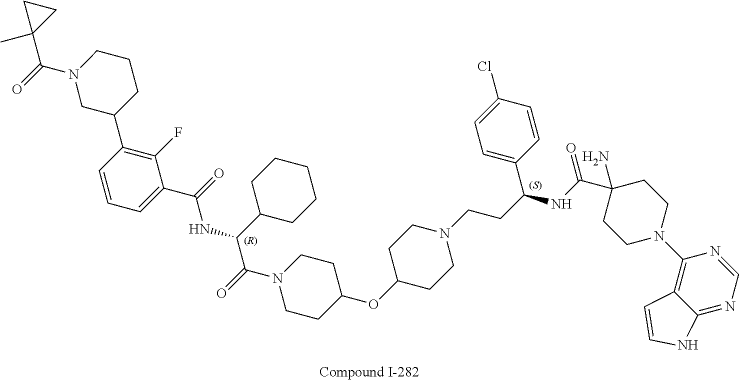

### Step 1: Synthesis of I-282-3.

To a mixture of I-282-1 (450 mg, 1.90 mmol, 1 eq) in DMF (4.5 mL) was added I-282-2 (189.88 mg, 1.90 mmol, 1 eq), EDCI (1.09 g, 5.69 mmol, 3 eq), HOAt (258.15 mg, 1.90 mmol, 265.31 μL, 1 eq) and NMM (959.17 mg, 9.48 mmol, 1.04 mL, 5 eq) at 25° C., the mixture was stirred at 25° C. for 1 hr. The reaction mixture was diluted with H₂O (5 mL) and extracted with DCM (5 mL×3), the combined organic layers were dried over Na₂SO₄, filtered, and concentrated to give a residue. The residue was purified by column chromatography (SiO₂, PE/EA=1/0 to 1/1) and concentrated to give the product. I-282-3 (650 mg, crude) was obtained as a yellow gum. LCMS: LCMS: R^{t}=0.365 min, [M+H]⁺=320.6.

### Step 2: Synthesis of I-282-4.

To a mixture of I-282-3 (600 mg, 1.88 mmol, 1 eq) in H₂O (2 mL), MeOH (2 mL) and THF (2 mL) was added LiOH·H₂O (236.51 mg, 5.64 mmol, 3 eq), the mixture was stirred at 25° C. for 1 hr. The reaction mixture was adjusted to pH=5 by 1M aq. HCl and extracted with DCM (5 mL×3), the combined organic layers were dried over Na₂SO₄, filtered, and concentrated to give product. I-282-4 (540 mg, crude) was obtained as a white solid. LCMS: LCMS: R^{t}=0.325 min, [M+H]⁺=306.1.

### Step 3: Synthesis of I-282-6.

To a solution of I-282-5 (315 mg, 377.48 μmol, 1 eq) in DMF (3.5 mL) was added I-282-4 (138.31 mg, 452.97 μmol, 1.2 eq), EDCI (217.09 mg, 1.13 mmol, 3 eq), HOAt (51.38 mg, 377.48 μmol, 52.80 μL, 1 eq) and NMM (190.90 mg, 1.89 mmol, 207.50 μL, 5 eq) at 25° C., the mixture was stirred at 25° C. for 1 hr. The reaction mixture was diluted with H₂O (5 mL) and extracted with DCM (5 mL×3), the combined organic layers were dried over Na₂SO₄, filtered and concentrated to give a residue. The residue was purified by reversed phase column (0.1% FA condition) and lyophilized to give the product. I-282-6 (320 mg, 277.79 umol, 73.59% yield, 97.384% purity) was obtained as a white solid. LCMS: R^{t}=0.380 min, [M+H]⁺=1122.1. SFC: R^{t}=1.862 min, 2.400 min.

### Step 4: Synthesis of 1-282.

To a mixture of I-282-6 (300 mg, 267.42 μmol, 1 eq) in DCM (3 mL) was added TFA (1 mL) at 25° C., the mixture was stirred at 25° C. for 0.5 hr. The reaction mixture was concentrated to give a residue. The residue was purified by reversed phase column (0.1% TFA condition) and lyophilized to give the product. I-282 (280.79 mg, 244.32 μmol, 91.36% yield, 98.82% purity, TFA) was obtained as a white solid. LCMS: LCMS: R^{t}=0.351 min, [M+H]⁺=1021.5. SFC: R^{t}=2.610 min, 3.225 min. ¹H NMR (400 MHZ, METHANOL-d4) δ=8.36 (d, J=2.0 Hz, 1H), 7.60-7.47 (m, 2H), 7.36 (d, J=2.0 Hz, 5H), 7.27 (t, J=7.6 Hz, 1H), 6.90 (t, J=3.6 Hz, 1H), 5.04-4.97 (m, 1H), 4.94 (d, J=7.6 Hz, 1H), 4.71-4.60 (m, 2H), 4.51 (d, J=13.6 Hz, 2H), 4.07-3.98 (m, 1H), 3.96-3.88 (m, 1H), 3.87-3.70 (m, 4H), 3.69-3.51 (m, 2H), 3.50-3.34 (m, 2H), 3.27-3.15 (m, 3H), 3.14-2.93 (m, 4H), 2.71-2.53 (m, 2H), 2.40-2.10 (m, 4H), 2.09-1.97 (m, 4H), 1.96-1.84 (m, 6H), 1.83-1.73 (m, 3H), 1.69 (d, J=13.2 Hz, 4H), 1.59-1.48 (m, 1H), 1.32 (s, 4H), 1.29-1.21 (m, 2H), 1.20-1.07 (m, 2H), 0.99-0.86 (m, 2H), 0.68-0.58 (m, 2H). ¹⁹F NMR (377 MHz, METHANOL-d4) δ=−77.11 (s, 7F), −121.66 (br d, J=19.8 Hz, 1F).

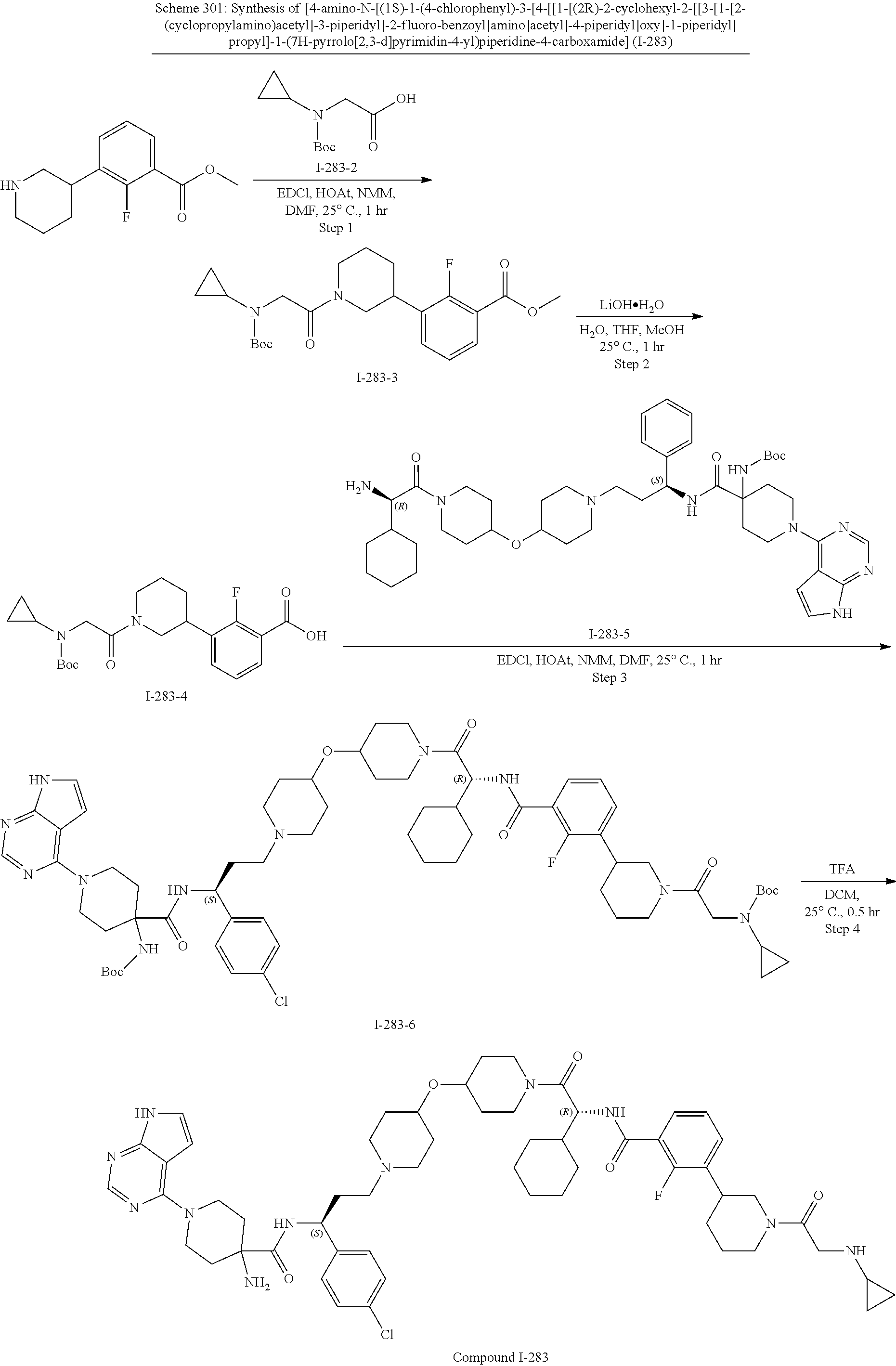

### Step 1: Synthesis of I-283-3.

To a solution of I-283-1 (480 mg, 1.75 mmol, 1 eq, HCl) in DMF (5 mL) was added I-283-2 (377.44 mg, 1.75 mmol, 1 eq), EDCI (1.01 g, 5.26 mmol, 3 eq), NMM (886.83 mg, 8.77 mmol, 963.95 μL, 5 eq) and HOAt (238.68 mg, 1.75 mmol, 245.30 μL, 1 eq). The mixture was stirred at 25° C. for 1 hr. The reaction mixture was diluted with H₂O (6 mL) and extracted with DCM (2 mL×3), the combined organic layers were dried over Na₂SO₄, filtered, and concentrated to give a residue. The residue was purified by flash silica gel chromatography (ISCO®; 20 g SepaFlash® Silica Flash Column, Eluent of 0˜60% Petroleum ether: Ethyl acetate @ 20 mL/min, PE/EA=0/1, Rf=0.6) and the eluent was concentrated to give the product. I-283-3 (754 mg, 1.71 mmol, 97.61% yield, 98.630% purity) was obtained as a yellow oil. LCMS: LCMS: R^{t}=0.454 min, [M+H]⁺=435.2.

### Step 2: Synthesis of I-283-4.

To a solution of I-283-3 (734 mg, 1.69 mmol, 1 eq) in H₂O (2.5 mL), THF (2.5 mL) and MeOH (2.5 mL) was added LiOH·H₂O (212.67 mg, 5.07 mmol, 3 eq). The mixture was stirred at 25° C. for 1 hr. The mixture was quenched by addition H₂O 8 mL at 25° C., and then adjusted to pH=5 with 1M aq. HCl, then extracted with DCM 9 mL (3 mL*3), dried over Na₂SO₄, filtered and concentrated under reduced pressure to give product. I-283-4 (510 mg, crude) was obtained as a white solid. LCMS: LCMS: R^{t}=0.419 min, [M-Boc+H]⁺=321.1.

### Step 3: Synthesis of I-283-6.

To a mixture of I-283-5 (315 mg, 377.48 μmol, 1 eq) in DMF (3.5 mL) was added I-283-4 (190.46 mg, 452.97 μmol, 1.2 eq), EDCI (217.09 mg, 1.13 mmol, 3 eq), HOAt (51.38 mg, 377.48 μmol, 52.80 μL, 1 eq) and NMM (190.90 mg, 1.89 mmol, 207.50 μL, 5 eq) at 25° C., the mixture was stirred at 25° C. for 1 hr. The reaction mixture was diluted with H₂O (5 mL) and extracted with DCM (5 mL×3), the combined organic layers were dried over Na₂SO₄, filtered, and concentrated to give a residue. The residue was purified by reversed phase column (neutral condition) and lyophilized to give the product. I-283-6 (280 mg, 222.51 μmol, 58.95% yield, 98.296% purity) was obtained as a white solid. LCMS: R^{t}=0.482 min, [M+H]/2+=619.3. SFC: R^{t}=1.772 min, 1.944 min.

### Step 4: Synthesis of I-283

To a mixture of I-283-6 (260 mg, 210.20 μmol, 1 eq) in DCM (3 mL) was added TFA (1 mL) at 25° C., the mixture was stirred at 25° C. for 0.5 hr. The reaction mixture was concentrated to give a residue. The residue was purified by reversed phase column (0.1% TFA condition) and lyophilized to give the product. I-283 (165.73 mg, 141.37 μmol, 67.26% yield, 98.16% purity; TFA) was obtained as a white solid. LCMS: R^{t}=0.302 min, 0.306 min, [M+H]⁺=1036.5. SFC: R^{t}=4.393 min, 5.430 min. ¹H NMR (400 MHz, METHANOL-d4) δ=8.36 (d, J=1.2 Hz, 1H), 7.65-7.48 (m, 2H), 7.42-7.31 (m, 5H), 7.31-7.23 (m, 1H), 6.93-6.87 (m, 1H), 5.04-4.93 (m, 2H), 4.67-4.54 (m, 3H), 4.33-4.18 (m, 2H), 4.05-3.95 (m, 1H), 3.95-3.89 (m, 1H), 3.89-3.72 (m, 5H), 3.69-3.42 (m, 3H), 3.38 (d, J=8.4 Hz, 1H), 3.28-3.13 (m, 4H), 3.12-2.98 (m, 2H), 2.92-2.74 (m, 2H), 2.72-2.54 (m, 2H), 2.41-2.30 (m, 1H), 2.30-2.11 (m, 3H), 2.10-1.98 (m, 4H), 1.97-1.90 (m, 3H), 1.89-1.74 (m, 6H), 1.70 (d, J=11.6 Hz, 4H), 1.60-1.49 (m, 1H), 1.36-1.21 (m, 3H), 1.20-1.08 (m, 2H), 0.96-0.87 (m, 4H). ¹⁹F NMR (376 MHz, METHANOL-d4) δ=−77.07 (s, 15F), −121.57-−122.04 (m, 1F).

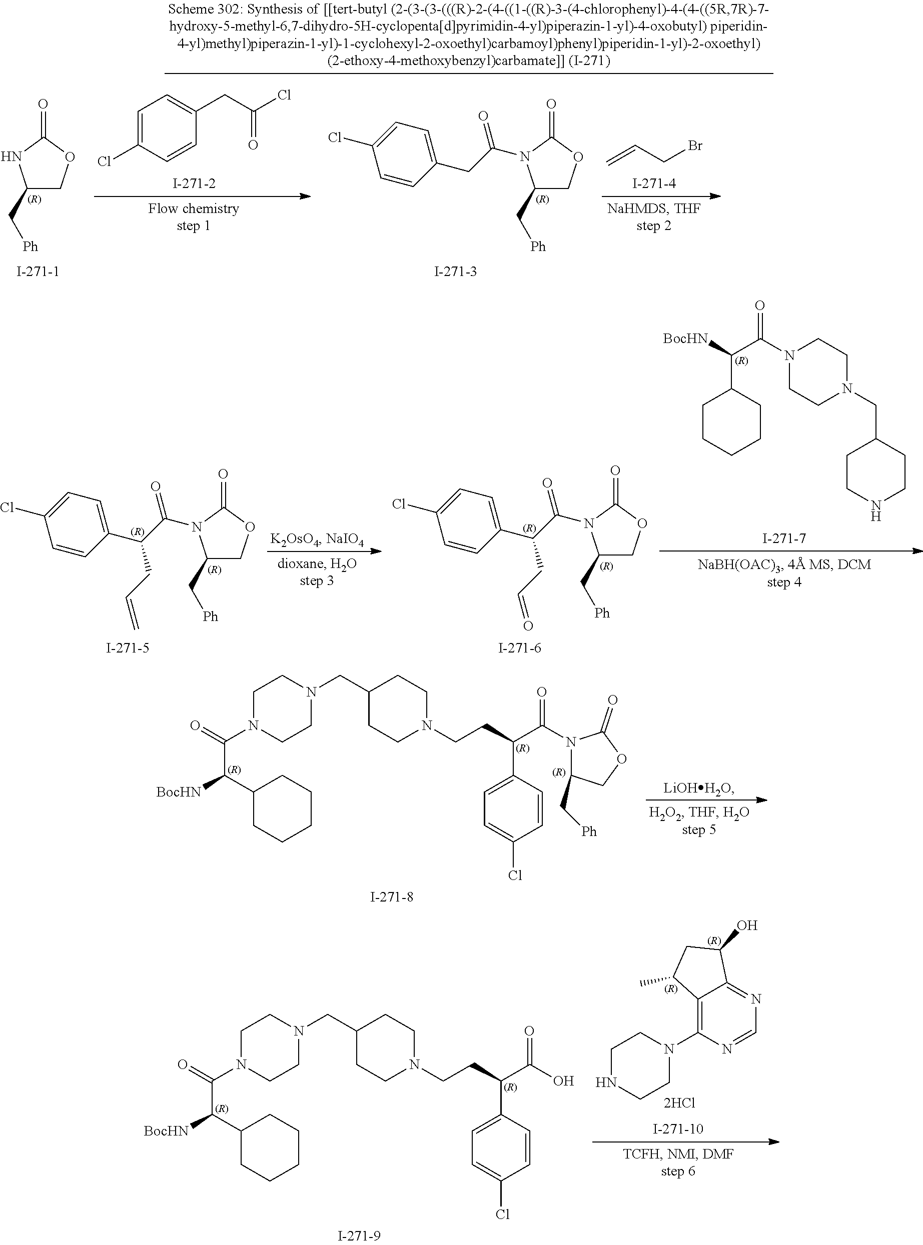

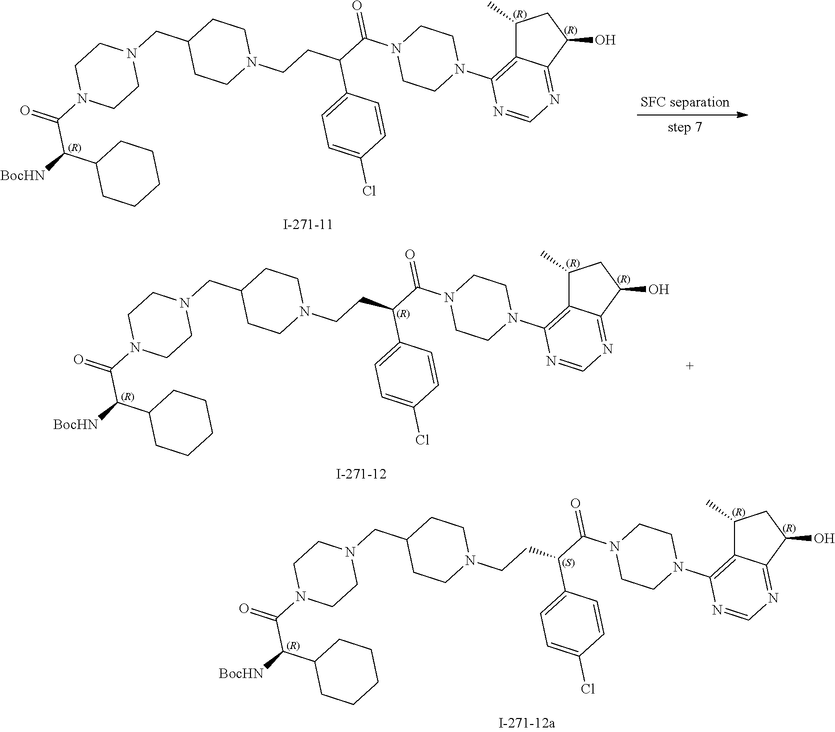

### Step 1: Synthesis of I-271-3

Solution 1: I-271-1, 1 eq, 16.4 g in THF, 328 mL. Solution 2: LiHMDS, 1.1 eq, 17.035 g. Solution 3: I-271-2, 1.3 eq, 22.744 g in THF, 328 mL. The volume of flow reactor 1FLR1, PFA, Coils reactor, 3.175 (1/8″) mm, 20.089 mL, 25° C. The volume of flow reactor 2FLR2, PFA, Coils reactor, 3.175 (⅛″) mm, 20.089 mL, 25° C. The residence time of flow reactor 1 was FLR1.1 min. The residence time of flow reactor 2 was FLR2, 0.559 min. Set the bath at {25° C.} for flow reactor 1. Set the bath at {25° C.} for flow reactor 2. The flow rate of Pump 1 was adjusted to S1, P1, 15.526 mL/min for solution 1. The flow rate of Pump 2 was adjusted to S2, P2, 4.563 mL/min for solution 2. The flow rate of Pump 3 was adjusted to S3, P3, 15.846 mL/min for solution 3. The mixture was collected with a bottle (contained 1000 mL aq. NH₄Cl (10%), 25° C.). The Pump 1 and Pump 2 was started at the same time. After 1 min, the Pump 3 was started. The reaction mixture was collected after running 0.599 mins. The reaction was diluted with aq. sat. NH₄Cl (50 mL), extracted with ethyl acetate 90 mL (30 mL*3), The organic phase was washed with saturated aqueous NaCl (50 mL). Then dried over [Na₂SO₄], filtered and concentrated under reduced pressure to give a residue. The mixture was purified by flash silica gel chromatography (ISCO®; 220 g SepaFlash® Silica Flash Column, Eluent of 0˜100% Ethyl acetate/Petroleum ether gradient @ 100 mL/min). The eluent was concentrated to afford desired product I-271-3 (12 g, 34.45 mmol, 94.66% purity) as white gum. LCMS: R^{t}=0.585 min, [M+H]⁺=330.1. ¹H NMR (400 MHZ, CHLOROFORM-d) δ=7.41-7.28 (m, 4H), 7.24 (s, 1H), 7.23 (br s, 4H), 4.70-4.62 (m, 1H), 4.33-4.11 (m, 4H), 3.24-3.20 (m, 1H), 2.75-2.65 (m, 1H).

### Step 2: Synthesis of I-271-5

To a solution of I-271-3 (9.8 g, 29.72 mmol, 142.86 μL, 1 eq) in THF (100 mL) was added NaHMDS (1 M, 35.66 mL, 1.2 eq) at −78° C., and was stirred at −78° C. for 0.5 hr under N₂. Then the reaction was added I-271-4 (14.38 g. 118.87 mmol, 10.27 mL, 4 eq) and was stirred at −78° C. for 0.5 h under N₂. Then the mixture was stirred at 25° C. for 11 hr under N₂. The reaction was diluted with aq. sat. NH₄Cl (100 mL), extracted with ethyl acetate 90 mL (30 mL*3), The organic phase was washed with saturated aqueous NaCl (100 mL). Then dried over [Na₂SO₄], filtered and concentrated under reduced pressure to give a residue. The mixture was purified by flash silica gel chromatography (ISCO®; 220 g SepaFlash® Silica Flash Column, Eluent of 0˜100% Ethyl acetate/Petroleum ether gradient @ 100 mL/min). The eluent was concentrated to afford desired product I-271-5 (9.25 g, 25.01 mmol, 84.16% yield, 100% purity) as white gum. LCMS: LCMS: R^{t}=0.635 min, [M+H]⁺=370.1.

### Step 3: Synthesis of I-271-6

To a solution of I-271-5 (9.25 g, 25.01 mmol, 1 eq) in dioxane (62 mL) and H₂O (31 mL) was added NaIO4 (16.05 g, 75.03 mmol, 4.16 mL, 3 eq) and K₂OsO₄ (921.53 mg, 2.50 mmol, 0.1 eq). The mixture was stirred at 25° C. for 1 hr. The reaction was poured into H₂O (50 mL). The mixture was extracted with DCM (25 mL*3). The combined organic layers were dried over Na₂SO₄, filtered and concentrated under reduced pressure to give a residue. The residue I-271-6 (9 g, 14.04 mmol, 56.13% yield, 58% purity) was obtained as a black oil and used into the next step without further purification. LCMS: LCMS: R^{t}=0.568 min, [M+H]⁺=372.1

### Step 4: Synthesis of I-271-8

To a solution of I-271-6 (4.4 g, 6.86 mmol, 1.2 eq) and I-271-7 (2.42 g, 5.72 mmol, 1 eq) in DCM (50 mL) was added 4 A MS (700 mg) and the mixture was stirred at 25° C. for 0.5 h, then NaBH(OAc)₃ (3.03 g. 14.30 mmol, 2.5 eq) was added to the mixture and the mixture was stirred at 25° C. for 0.5 hrs. The reaction mixture was filtered to remove solid. The filtrate was poured into 10% K₂CO₃ aqueous (10 mL), then extracted by DCM (10 mL×3). The combined organic layer was washed with brine (10 mL), dried over anhydrous sodium sulfate, filtered and concentrated to give residue. The residue was purified by reversed phase column (26.8*125 mm, of XB—C18, 20-40 μm, 80 Å) Mobile phase: A for H₂O (0.1% FA v/v) and B for acetonitrile; Gradient: B 0%-100% in 20 min: Flow rate: 50 mL/min; Column temperature: R.T. Wavelength: 220 nm/254 nm), then concentrated to remove organic solvents and lyophilized to give product. I-271-8 (3 g, 2.54 mmol, 44.47% yield, 66% purity) was obtained as a white solid. LCMS: R^{t}=0.492 min, [M+H]⁺=778.5.

### Step 5: Synthesis of I-271-9

To a solution of LiOH·H₂O (323.45 mg, 7.71 mmol, 2 eq) in THF (30 mL) and H₂O (10 mL) was added in H₂O₂ (1.31 g, 11.56 mmol, 1.11 mL, 30% purity; 3 eq) at 0° C. The mixture was stirred at 0° C. for 0.5 hr. Then I-271-8 (3 g, 3.85 mmol, 1 eq) in THF (30 mL) was added in and the mixture was stirred at 0° C. for 0.5 hr. Then the mixture was stirred at 25° C. for 11 hr. The reaction mixture was cooled to 0° C. again and treated with 1 M Na₂SO₃ (20 mL). The aqueous layer was acidified with solid KHCO₃ and extracted with DCM (2×20 mL). The combined organic layers were dried over Na₂SO₄, filtered and concentrated under reduced pressure to give a residue. The product was purified by prep-HPLC (column: Waters Xbridge 150*25 mm*5 um; mobile phase: [water (NH₄HCO₃)-ACN]; gradient: 28%-58% B over 9 min) and the eluent was concentrated to remove MeCN and then lyophilization to get crude product. Then crude product was purified by prep-HPLC (column: Welch Ultimate XB—CN 250*70*10 um; mobile phase: [Hexane-EtOH]; gradient: 20%-60% B over 15 min) and the eluent was concentrated to get product. I-271-9 (1 g, 1.57 mmol, 40.65% yield, 97% purity) was obtained as a black solid. LCMS: LCMS: R^{t}=0.442 min, [M+H]⁺=619.3. SFC: R^{t}=1.748 min, cc %=100%.

### Step 6: Synthesis of I-271-11

To a solution of I-271-9 (800 mg, 1.29 mmol, 1 eq) and I-271-10 (476.28 mg, 1.55 mmol, 1.2 eq, 2HCl) in DMF (8 mL) was added TCFH (1.09 g, 3.88 mmol, 3 eq) and NMI (530.33 mg, 6.46 mmol, 514.89 μL, 5 eq). The mixture was stirred at 25° C. for 1 hr. The reaction was diluted with H₂O (3 mL), extracted with ethyl acetate 9 mL (3 mL*3), The organic phase was washed with saturated aqueous NaCl (5 mL). Then dried over Na₂SO₄, filtered and concentrated under reduced pressure to give a residue. The mixture was purified by flash silica gel chromatography (ISCO®; 80 g SepaFlash® Silica Flash Column, Eluent of 0˜100% Ethyl acetate/MeOH @ 100 mL/min). The eluent was concentrated to afford desired product I-271-11 (800 mg, 894.20 μmol, 69.22% yield, 93.39% purity) as white gum. LCMS: LCMS: R^{t}=0.442 min, [M+H]⁺=835.7.

### Step 7: Synthesis of I-271-12 and I-271-12a

The mixture of I-271-11 was separated by SFC (column: (s,s) WHELK-01 (250 mm*30 mm, 10 um); mobile phase: [CO₂-ACN/i-PrOH (0.1% NH₃H₂O)]: B %: 50%, isocratic elution mode) and the eluent was concentrated to give the product. I-271-12 (350 mg, 418.90 μmol, 43.75% yield, 100% purity) was obtained as a yellow solid and I-271-12a (350 mg, 410.53 μmol, 42.87% yield, 98% purity) was obtained as a yellow solid. LCMS: R^{t}=0.418 min, [M+H]⁺=835.5. SFC (12): R^{t}=0.866 min, ee value>99%. SFC (12a): R^{t}=1.211 min, ee value=98.62%.

### Step 8: Synthesis of I-271-13

To a solution of I-271-12 (270 mg, 323.15 μmol, 1 eq) in DCM (3 mL) was added HCl/dioxane (2 M, 2.70 mL, 16.71 eq). The mixture was stirred at 25° C. for 1 hr. The reaction mixture was concentrated under reduced pressure to give a residue. The crude product I-271-13 (270 mg, crude, HCl) was obtained as yellow solid and used in the next step without further purification. LCMS: R^{t}=0.365 min, [M+H]⁺=735.5

### Step 9: Synthesis of I-271-15

To a solution of I-271-13 (100 mg, 129.56 μmol, 1 eq, HCl) and I-271-14 (68.23 mg, 129.56 μmol, 1 eq) in DMF (1 mL) was added EDCI (74.51 mg, 388.67 μmol, 3 eq), HOAt (17.63 mg, 129.56 μmol, 18.12 μL, 1 eq) and NMM (131.04 mg, 1.30 mmol, 142.44 μL, 10 eq). The mixture was stirred at 25° C. for 1 hr. The reaction was diluted with H₂O (5 mL), extracted with ethyl acetate 9 mL (3 mL*3), The organic phase was washed with saturated aqueous NaCl (5 mL). Then dried over Na₂SO₄, filtered and concentrated under reduced pressure to give a residue. The mixture was purified by flash silica gel chromatography (ISCO®; 12 g SepaFlash® Silica Flash Column, Eluent of 0˜100% Ethyl acetate/MeOH @ 20 mL/min). The eluent was concentrated to afford desired product. I-271-15 (100 mg, 75.56 μmol, 58.32% yield, 94% purity) was obtained as white solid. LCMS: R^{t}=0.500 min, [M+H]⁺=1243.6. SFC: R^{t}=1.417 min and 1.469 min.

### Step 10: Synthesis of I-271

To a solution of I-271-15 (100 mg, 80.39 μmol, 1 eq) in DCM (1 mL) was added 2,6-lutidine (4.31 mg, 40.19 μmol, 4.68 μL, 0.5 eq) and TMSOTf (26.80 mg, 120.58 μmol, 21.79 μL, 1.5 eq). The mixture was stirred at 0° C. for 0.5 hr. The reaction mixture was concentrated to give the crude product. The crude product was purified by prep-HPLC (column: YMC-Actus Triart C18 150*30 mm*7 um; mobile phase: [water (FA)-ACN]; gradient: 13%-43% B over 10 min) and the eluent was concentrated to remove MeCN and then lyophilization to get crude product. Then the crude product was purified by prep-HPLC (column: Phenomenex luna C18 150*25 mm*10 um; mobile phase: [water (FA)-ACN]; gradient: 8%-38% B over 11 min) and the eluent was concentrated to remove MeCN and then lyophilization to get product. I-271 (14.39 mg, 12.09 μmol, 15.04% yield, 100% purity, FA) was obtained as a white solid. LCMS: R^{t}=0.425 min, [M+H]⁺=1144.0. SFC: R^{t}=4.705 min and 4.940 min. ¹H NMR (400 MHZ, METHANOL-d4) δ=8.55 (s, 1H), 8.43 (s, 1H), 7.77-7.68 (m, 2H), 7.50-7.40 (m, 2H), 7.37-7.31 (m, 4H), 7.20-7.16 (m, 1H), 6.59-6.46 (m, 2H), 4.98-4.95 (m, 1H), 4.58-4.46 (m, 1H), 4.15-4.02 (m, 3H), 3.95-3.63 (m, 16H), 3.62-3.47 (m, 5H), 3.19-3.04 (m, 4H), 2.87-2.65 (m, 2H), 2.62-2.31 (m, 7H), 2.23 (d, J=7.2 Hz, 4H), 2.18-2.10 (m, 1H), 2.09-1.99 (m, 2H), 1.96-1.74 (m, 9H), 1.73-1.55 (m, 4H), 1.47-1.39 (m, 3H), 1.35-1.22 (m, 5H), 1.17-1.05 (m, 5H).

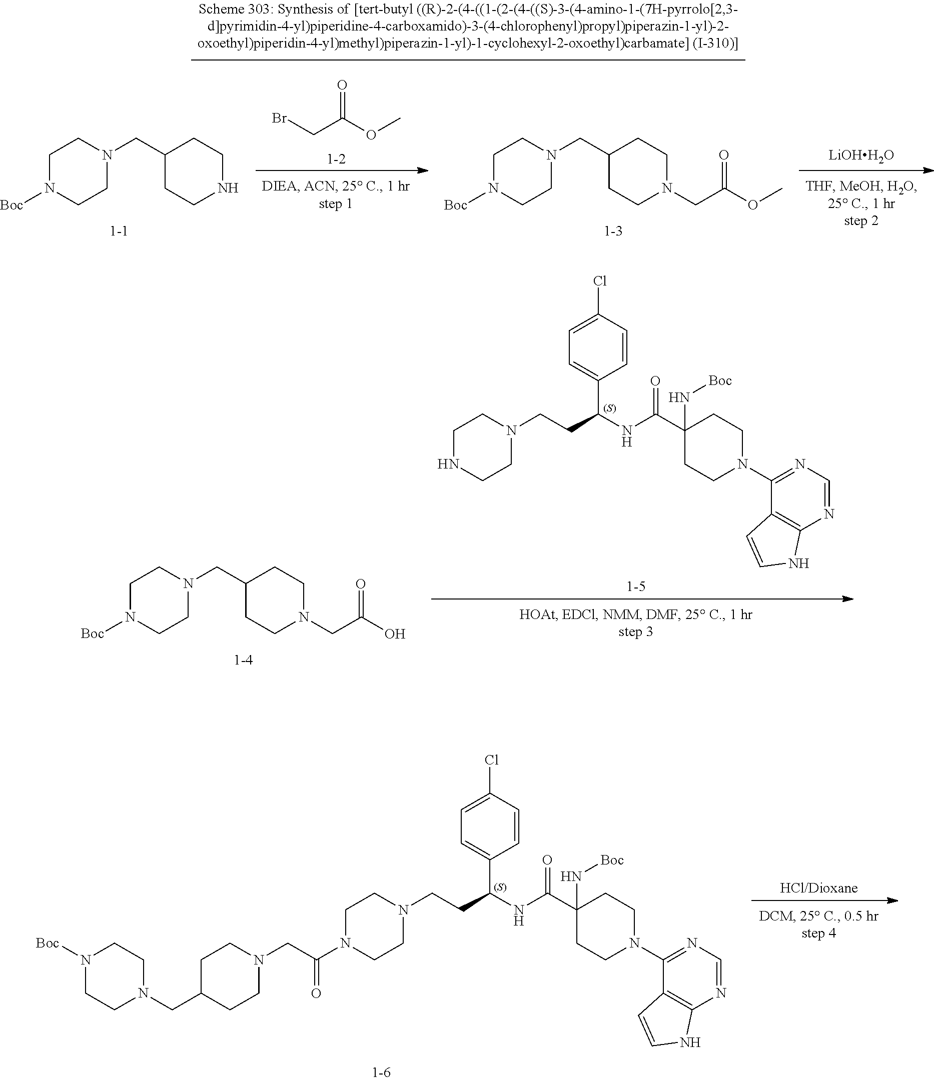

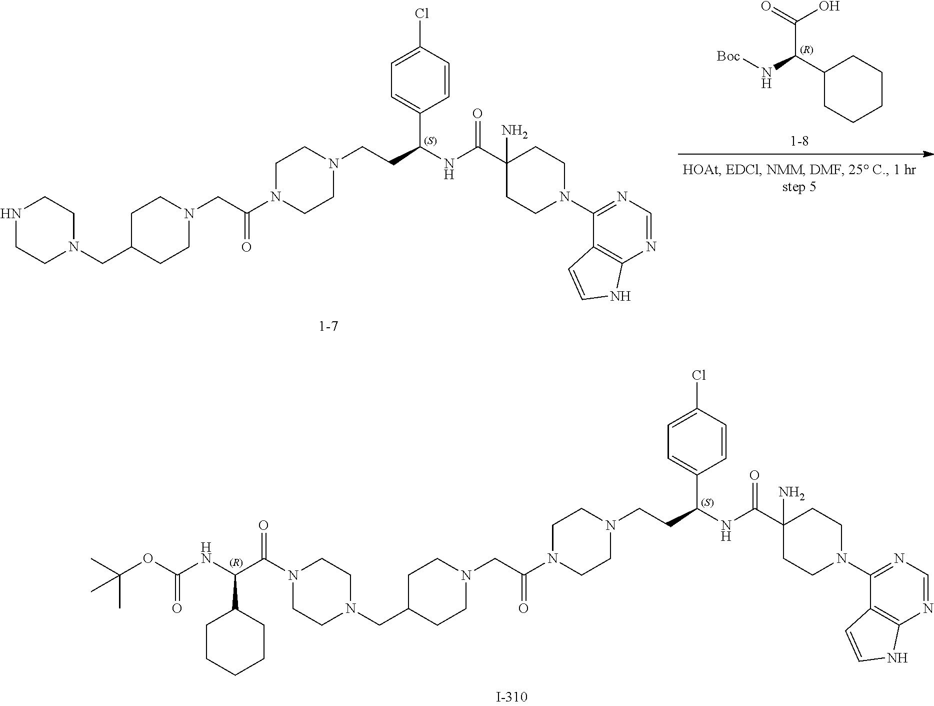

To a solution of 1-1 (1.5 g, 5.29 mmol, 1 eq) and 1-2 (809.65 mg, 5.29 mmol, 501.02 μL, 1 eq) in ACN (15 mL) was added DIEA (2.05 g, 15.88 mmol, 2.77 mL, 3 eq), then the mixture was stirred at 25° C. for 1 hr. The reaction mixture was diluted with water (50 mL) and extracted with EA 60 mL (20 mL*3). The combined organic layers were dried over Na₂SO₄, filtered and concentrated under reduced pressure to give a residue. 1-3 (2 g, crude) was obtained as a white solid. LCMS: Rt: 0.124 min, [M+H]⁺=356.3

### Step 2: Synthesis of 1-4

To a solution of 1-3 (1.3 g, 3.66 mmol, 1 eq) in THF (5 mL), MeOH (5 mL), H₂O (5 mL) was added LiOH·H₂O (460.40 mg, 10.97 mmol, 3 eq). The mixture was stirred at 20° C. for 1 hr. The mixture was adjusted to pH=7 with 1 N HCl and extracted with EA 120 mL (40 mL*3). The combined organic layers were lyophilized to give the product. 1-4 (1.4 g, crude) was obtained as a white solid. LCMS: Rt: 0.123 min, [M+H]⁺=342.2.

### Step 3: Synthesis of 1-6

To a solution of 1-5 (400 mg, 1.17 mmol, 1 eq) and 1-4 (699.56 mg, 1.17 mmol, 1 eq) in DMF (5 mL) was added HOAt (159.45 mg, 1.17 mmol, 163.88 μL, 1 eq), EDCI (673.73 mg, 3.51 mmol, 3 eq) and NMM (592.47 mg, 5.86 mmol, 643.98 μL, 5 eq). The mixture was stirred at 25° C. for 0.5 hr. The reaction mixture was diluted with H₂O 10 mL and extracted with EA 45 mL (15 mL*3). The combined organic layers were washed with brine 30 mL (10 mL*3), dried over [Na₂SO₄], filtered and concentrated under reduced pressure to give a residue. The residue was purified by flash silica gel chromatography (ISCO®; 20 g SepaFlash® Silica Flash Column, Eluent of 0˜15% Methanol/Dichloromethane gradient (a, 60 mL/min), the eluent was concentrated under reduced pressure to give a residue. 1-6 (800 mg, 829.32 μmol, 70.79% yield, 95.432% purity) was obtained as a yellow oil. LCMS: Rt: 0.272 min, [M+H]⁺=920.5 SFC: Retention time: 1.270 min. ¹H NMR (400 MHZ, METHANOL-d4) δ=8.13 (s, 1H), 7.37-7.24 (m, 4H), 7.12 (d, J=3.6 Hz, 1H), 6.62 (d, J=3.6 Hz, 1H), 5.06-4.98 (m, 1H), 4.37 (d, J=4.0 Hz, 2H), 3.63 (s, 6H), 3.45-3.36 (m, 6H), 3.07-2.89 (m, 2H), 2.54-2.30 (m, 11H), 2.21 (d, J=7.2 Hz, 3H), 2.17-1.92 (m, 6H), 1.80 (d, J=12.4 Hz, 2H), 1.50-1.38 (m, 18H), 1.35-1.21 (m, 3H).

### Step 4: Synthesis of 1-7

To a solution of 1-6 (600 mg, 651.76 μmol, 1 eq) in DCM (4 mL) was added HCl/dioxane (2 M, 4 mL, 12.27 eq). The mixture was stirred at 25° C. for 1 hr. The reaction mixture was concentrated under reduced pressure to give a residue. The crude product was used into the net step without further purification. 1-7 (550 mg, crude, HCl) was obtained as a white solid. LCMS: Rt: 0.194 min, [M+H]⁺=720.4

### Step 5: Synthesis of I-310

To a solution of 1-8 (150 mg, 582.92 μmol, 1 eq) and 1-7 (441.16 mg, 582.92 μmol, 1 eq, HCl) in DMF (5 mL) was added HOAt (79.34 mg, 582.92 μmol, 81.54 μL, 1 eq), EDCI (335.24 mg, 1.75 mmol, 3 eq) and NMM (294.80 mg, 2.91 mmol, 320.44 μL, 5 eq). The mixture was stirred at 25° C. for 0.5 hr. The reaction mixture was diluted with H₂O 10 mL and extracted with EA 45 mL (15 mL*3). The combined organic layers were washed with brine 45 mL (15 mL*3), dried over Na₂SO₄, filtered and concentrated under reduced pressure to give a residue. The crude product was purified by reversed-phase column (0.1% FA condition), the eluent was concentrated and lyophilized to give impure product. The product was further purified by Prep-HPLC (26.8*125 mm, 80 g of XB—C18, 20-40 μm, 120 Å) Mobile phase: A for H2O (0.1% NH₃·H2O v/v) and B for acetonitrile; Gradient: B 5%-95% in 15 min; Flow rate: 40 mL/min; Column temperature: R.T. Wavelength: 220 nm/254 nm), after purification, the target peak was concentrated and lyophilized to afford desired product. I-310 (75.37 mg, 77.65 μmol, 18.63% yield, 98.87% purity) was obtained as a white solid. LCMS: R^{t}=10.095 min, [M+H]⁺=959.6 ¹H NMR (400 MHZ, DMSO-d₆) δ=11.65 (s, 1H), 8.75 (d, J=8.0 Hz, 1H), 8.11 (s, 1H), 7.38-7.30 (m, 4H), 7.15 (s, 1H), 6.74 (d, J=8.8 Hz, 1H), 6.57 (s, 1H), 6.45-6.34 (m, 1H), 4.90-4.80 (m, 1H), 4.42-4.36 (m, 2H), 4.22-4.20 (m, 1H), 4.13-4.04 (m, 1H), 3.61-3.41 (m, 10H), 3.11-3.02 (m, 2H), 2.75 (d, J=10.0 Hz, 2H), 2.39-2.26 (m, 6H), 2.25-2.16 (m, 5H), 2.09 (d, J=6.6 Hz, 2H), 2.01-1.83 (m, 6H), 1.72-1.55 (m, 7H), 1.53-1.40 (m, 4H), 1.35 (s, 9H), 1.17-1.01 (m, 5H), 0.98-0.86 (m, 2H). SFC: R^{t}=1.598 min, (ee %=100%).

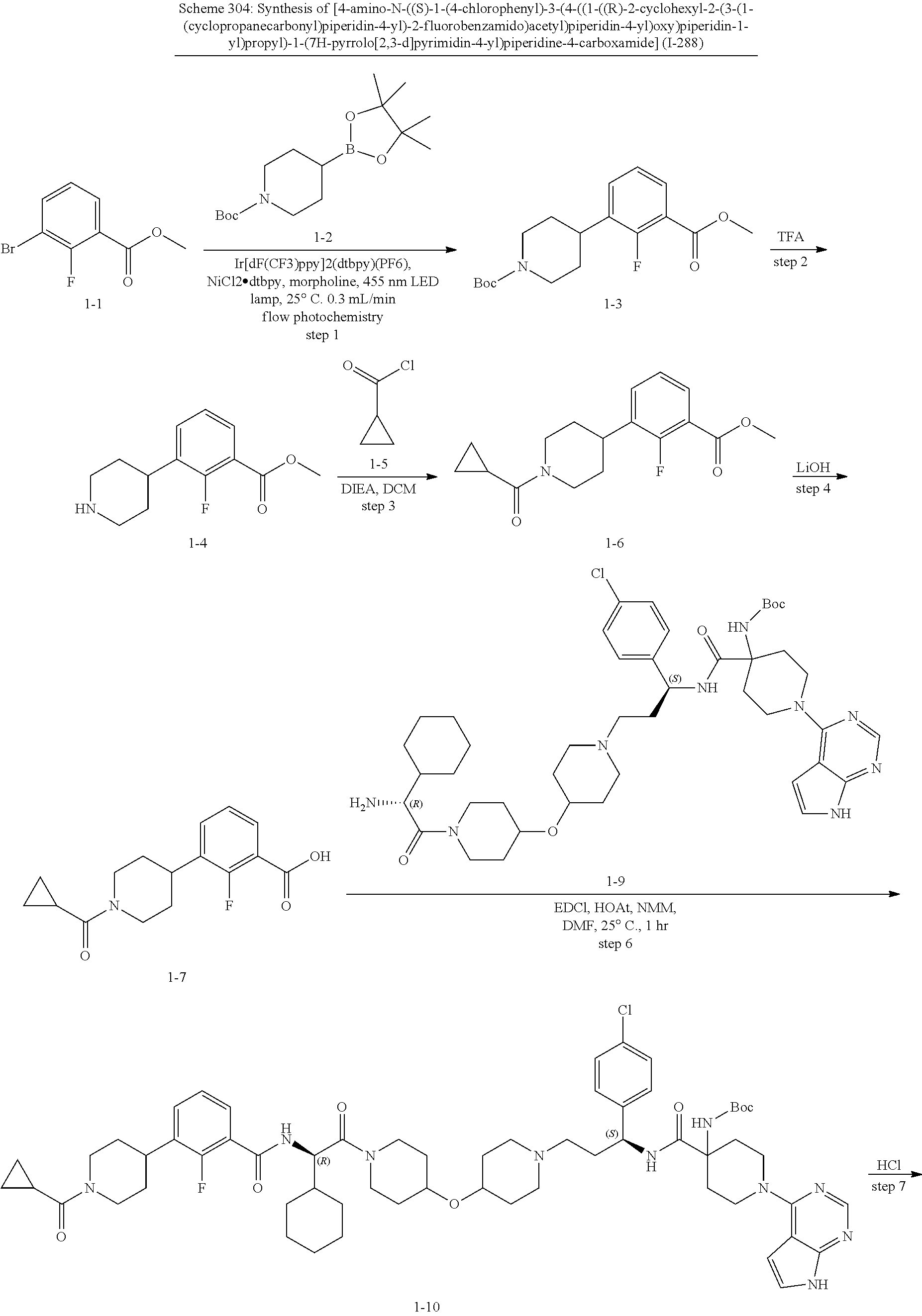

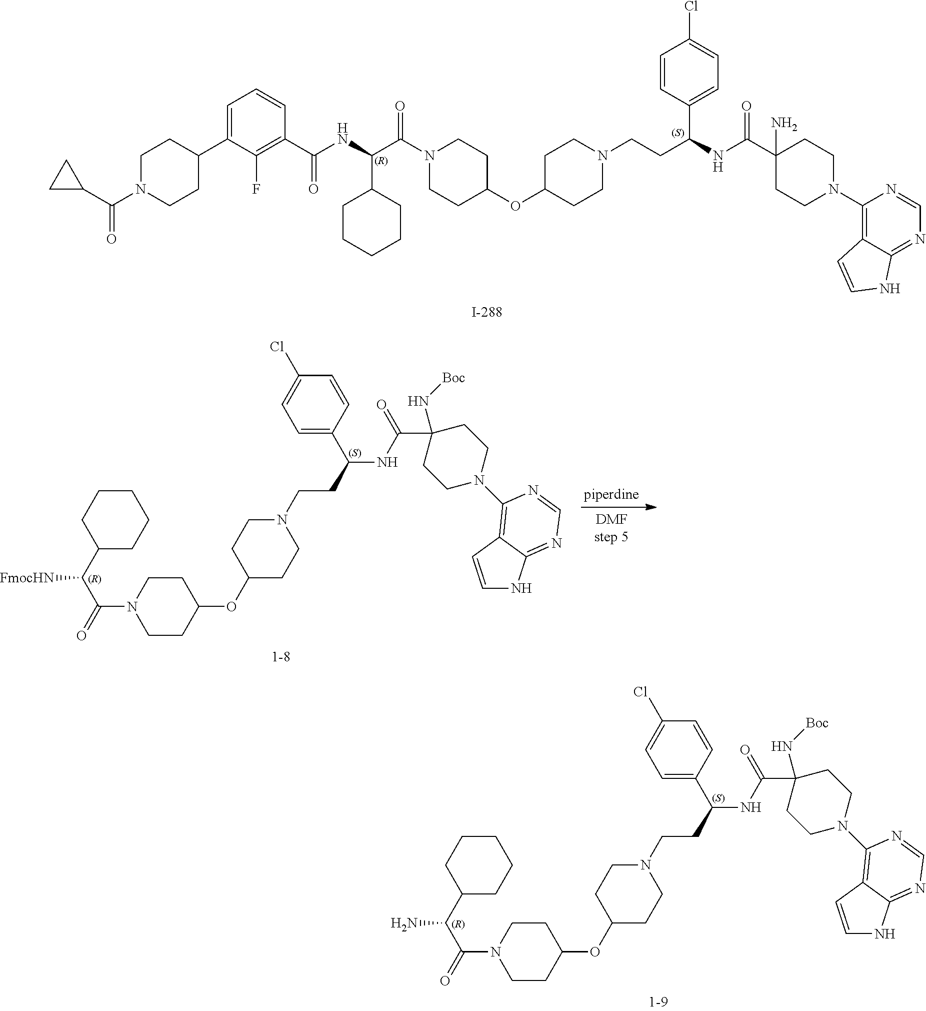

To an 15 mL vial equipped with a stir bar was added 1-1 (0.2 g, 858.24 μmol, 1 eq), 1-2 (534.21 mg, 1.72 mmol, 2 eq), bis [3,5-difluoro-2-[5-(trifluoromethyl)-2-pyridyl]phenyl]iridium (1+); 4-tert-butyl-2-(4-tert-butyl-2-pyridyl)pyridine; hexafluorophosphate (9.63 mg, 8.58 μmol, 0.01 eq), 4-tert-butyl-2-(4-tert-butyl-2-pyridyl)pyridine; dichloronickel (17.08 mg, 42.91 μmol, 0.05 eq), morpholine (112.16 mg, 1.29 mmol, 113.29 μL, 1.5 eq) in DMF (4 mL). The reaction solution was pumped through the reactor at a flow rate of 300 μL min⁻¹, irradiating with a 455 nm LED lamp with the flow wizard program, with cooling water to keep the reaction temperature at 25° C. for 0.5 h. The reaction mixture was filtered and concentrated under reduced pressure to give a residue. The residue was purified by reversed-phase column (0.1% FA) the eluent was concentrated to remove ACN and lyophilized to get product, Then the residue was purified by flash silica gel chromatography (ISCO®; 20 g SepaFlash® Silica Flash Column, Eluent of 0˜60% Ethyl acetate/Petroleum ether gradient (@ 30 mL/min). The eluent was concentrated to get 1-3 (100 mg, 296.40 μmol, 34.54% yield) as colorless oil. LCMS: Retention time: 0.468 min, [M+H]⁺=282.0. ¹H NMR (400 MHz, METHANOL-d4) δ=7.80-7.69 (m, 1H), 7.58-7.45 (m, 1H), 7.24-7.20 (m, 1H), 4.23 (d, J=13.6 Hz, 2H), 3.90 (s, 3H), 3.16-3.09 (m, 1H), 2.90 (s, 2H), 1.81 (d, J=12.8 Hz, 2H), 1.70-1.59 (m, 2H), 1.48 (s, 9H).

### Step 2: Synthesis of 1-4

A solution of 1-3 (0.1 g, 296.40 μmol, 1 eq) in HCl/dioxane (1 mL). The mixture was stirred at 25° C. for 0.5 hr. The reaction mixture was concentrated under reduced pressure to give 1-4 (0.1 g, crude, HCl) was obtained as a white solid. LCMS: Retention time: 0.259 min, [M+H]⁺=237.9.

### Step 3: Synthesis of 1-6

To a solution of 1-4 (0.1 g, 365.32 μmol, 1 eq, HCl) in DCM (1 mL) was added DIEA (141.65 mg, 1.10 mmol, 190.90 μL, 3 eq), then added 1-5 (45.83 mg, 438.39 μmol, 39.78 μL, 1.2 eq). The mixture was stirred at 25° C. for 1 hr. The reaction mixture was concentrated under reduced pressure to give a residue. The residue was purified by flash silica gel chromatography (ISCO®; 12 g SepaFlash® Silica Flash Column, Eluent of 0˜100% Ethyl acetate/Petroleum ether gradient @ 20 mL/min) was concentrated under reduced pressure to give 1-6 (0.1 g, 327.50 μmol, 89.65% yield) was obtained as a white solid. LCMS: Retention time: 0.431 min, [M+H]⁺=306.3.

### Step 4: Synthesis of 1-7

To a solution of 1-6 (0.1 g, 327.50 μmol, 1 eq) in H₂O (0.5 mL) was added LiOH·H₂O (41.23 mg, 982.50 μmol, 3 eq) MeOH (0.5 mL), THF (0.5 mL). The mixture was stirred at 25° C. for 0.5 hr. The reaction mixture was adjusted pH=6 by HCl (2M), and then extracted with DCM 10 mL (5 mL*2). The combined organic layers were washed with NaCl 5 mL, dried over Na₂SO₄, filtered and concentrated under reduced pressure to give 1-7 (0.1 g, crude) was obtained as a white solid. LCMS: Retention time: 0.357 min, (M+H)=291.9.

### Step 5: Synthesis of 1-9

To a solution of 1-8 (0.1 g, 94.63 μmol, 1 eq) in DMF (1 mL) was added piperidine (24.17 mg, 283.90 μmol, 28.04 μL, 3 eq). The mixture was stirred at 25° C. for 0.5 hr. The reaction mixture was diluted with H₂O 2 mL and extracted with DCM 10 mL (5 mL*2). The combined organic layers were washed with NaCl 10 mL (5 mL*2), dried over Na₂SO₄, filtered and concentrated under reduced pressure to give a residue. 1-9 (0.1 g, crude) was obtained as a yellow oil. LCMS: Retention time: 0.283 min, [M+H]⁺=834.5.

### Step 6: Synthesis of 1-10

To a solution of 1-7 (37.70 mg, 129.42 μmol, 1.2 eq), 1-9 (90 mg, 107.85 μmol, 1 eq) in DMF (1.5 mL) was added EDCI (62.03 mg, 323.55 μmol, 3 eq) and HOAt (14.68 mg, 107.85 μmol, 15.09 μL, 1 eq), NMM (54.54 mg, 539.25 μmol, 59.29 μL, 5 eq). The mixture was stirred at 25° C. for 0.5 hr. The reaction mixture was filtered and concentrated under reduced pressure to give a residue. The residue was purified by prep-HPLC (column: CD06-Waters Xbidge C18 150*40*10 um; mobile phase: [water (HCl)-ACN]; gradient: 31%-61% B over 45 min) the eluent was concentrated to remove ACN and lyophilized to get product. 1-10 (40 mg, 36.11 μmol, 33.48% yield) was obtained as a white solid. LCMS: Retention time: 0.845 min, [M+H]⁺=1107.8.

### Step 7: Synthesis of I-288

A solution of 1-10 (25 mg, 22.57 μmol, 1 eq) in HCl/dioxane (0.3 mL). The mixture was stirred at 25° C. for 0.15 hr. The reaction mixture was concentrated under reduced pressure to give a residue. The residue was lyophilized to get product. I-288 (20.91 mg, 19.38 μmol, 85.88% yield, 96.783% purity, HCl) was obtained as a white solid. LCMS: Retention time: 0.369 min, [M+H]⁺=1007.9. SFC: Retention time: 1.189 min. ¹H NMR (400 MHZ, METHANOL-d4) δ=8.40 (s, 1H), 7.56-7.52 (m, 1H), 7.50-7.45 (m, 1H), 7.45-7.39 (m, 3H), 7.38-7.33 (m, 2H), 7.27-7.21 (m, 1H), 7.03-6.99 (m, 1H), 5.04-4.99 (m, 1H), 4.97-4.94 (m, 1H), 4.73-4.59 (m, 3H), 4.50 (d, J=12.8 Hz, 1H), 4.06-3.98 (m, 1H), 3.97-3.85 (m, 3H), 3.83-3.72 (m, 2H), 3.68-3.52 (m, 2H), 3.51-3.39 (m, 2H), 3.29-3.20 (m, 4H), 3.16-3.02 (m, 2H), 2.90-2.69 (m, 3H), 2.56-2.41 (m, 1H), 2.34-2.15 (m, 3H), 2.10-1.92 (m, 7H), 1.90-1.82 (m, 4H), 1.77 (d, J=12.4 Hz, 3H), 1.73-1.63 (m, 4H), 1.38-1.21 (m, 4H), 1.18-1.06 (m, 2H), 0.93-0.87 (m, 2H), 0.83 (d, J=7.6 Hz, 2H). ¹⁹F NMR (377 MHz, METHANOL-d4) δ=−116.23-−127.61 (m, 1F).

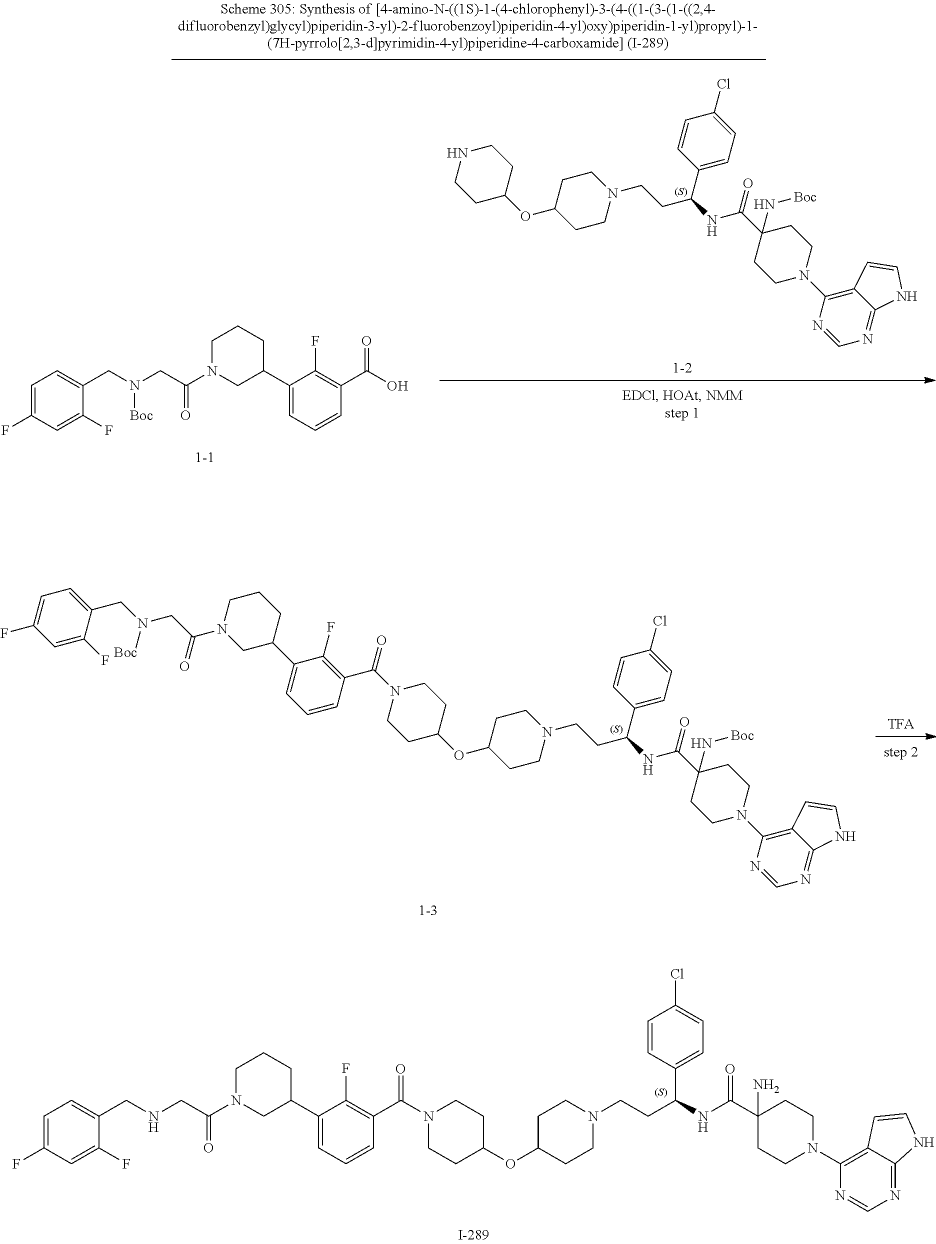

To a solution of 1-1 (43.71 mg, 86.29 μmol, 1.2 eq), 1-2 (50 mg, 71.91 μmol, 1 eq) in DMF (1 mL) was added HOAt (9.79 mg, 71.91 μmol, 10.06μ, 1 eq) and NMM (36.37 mg, 359.56 μmol, 39.53μ, 5 eq), EDCI (41.36 mg, 215.74 μmol, 3 eq). The mixture was stirred at 25° C. for 0.5 hr. The reaction mixture was diluted with H₂O 2 mL and extracted with DCM 10 mL (5 mL*2). The combined organic layers were washed with NaCl 10 mL (5 mL*2), dried over Na₂SO₄, filtered and concentrated under reduced pressure to give a residue. The residue was purified by reversed phase (0.1% NH3·H2O) the eluent was concentrated to remove ACN and lyophilized to get product. 1-3 (50 mg, 38.86 μmol, 54.04% yield, 92.012% purity) was obtained as a white solid. LCMS: Retention time: 2.913 min, [M/2+H]⁺=592.8. SFC: Retention time: 3.706 min, 4.197 min.

### Step 2: Synthesis of I-289

To a solution of 1-3 (50 mg, 42.24 μmol, 1 eq) in DCM (0.5 mL) was added TFA (153.50 mg, 1.35 mmol, 100.00 μL, 31.87 eq). The mixture was stirred at 25° C. for 0.5 hr. The reaction mixture was concentrated under reduced pressure to give a residue. The residue was purified by prep-HPLC (column: CD04-Welch Ultimate C18 150*25*7 um; mobile phase: [water (FA)-ACN]; gradient: 2%-32% B over 10 min) the eluent was concentrated to remove ACN and lyophilized to get product. I-289 (29.36 mg, 28.16 μmol, 66.68% yield, 98.766% purity, FA) was obtained as a white solid. LCMS: Retention time: 0.718 min, [M+H]⁺=983.4. SFC: Retention time: 1.350 min, 1.560 min. ¹H NMR (400 MHZ, METHANOL-d4) 8=8.32 (d, J=2.4 Hz, 1H), 7.66-7.57 (m, 1H), 7.51-7.43 (m, 1H), 7.36 (s, 4H), 7.33-7.24 (m, 3H), 7.18-7.07 (m, 2H), 6.86-6.82 (m, 1H), 5.02-4.98 (m, 1H), 4.66-4.54 (m, 3H), 4.34-4.30 (m, 2H), 4.19 (d, J=12.8 Hz, 1H), 4.15-4.08 (m, 1H), 4.06-3.88 (m, 2H), 3.84-3.69 (m, 5H), 3.59-3.47 (m, 2H), 3.24-3.13 (m, 5H), 3.11-2.97 (m, 3H), 2.90 (s, 1H), 2.87-2.72 (m, 1H), 2.67-2.50 (m, 2H), 2.34 (d, J=12.0 Hz, 1H), 2.27-2.19 (m, 1H), 2.12 (d, J=14.8 Hz, 1H), 2.07-1.99 (m, 3H), 1.98-1.88 (m, 5H), 1.85-1.79 (m, 1H), 1.72-1.63 (m, 2H), 1.58-1.49 (m, 1H). ¹⁹F NMR (376 MHz, METHANOL-d4) δ=−77.01 (br s, 15F), −108.36 (br s, 1F), −112.78-−112.92 (m, 1F), −122.14-−126.92 (m, 1F).

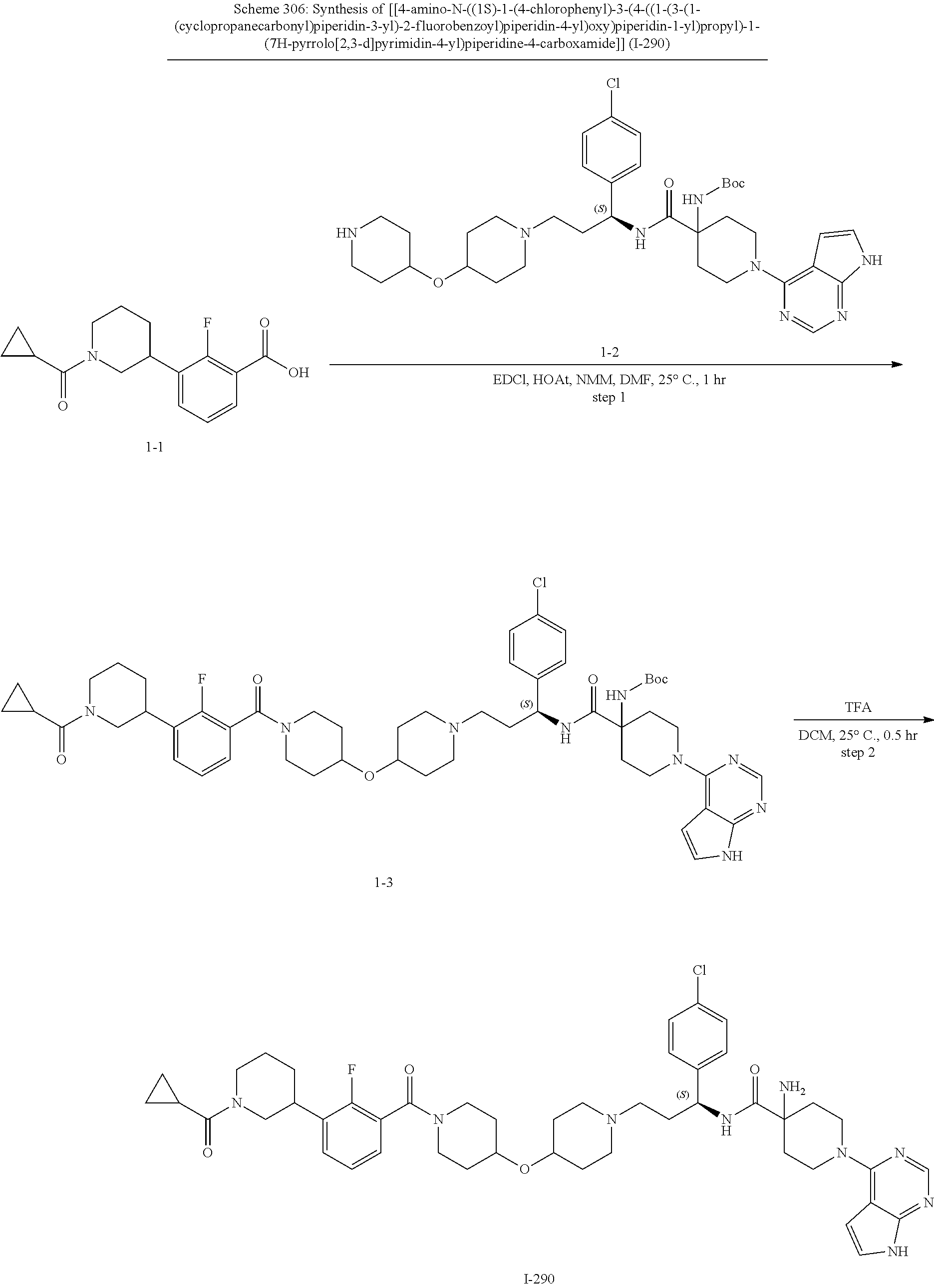

### Step 1: Synthesis of 1-3

To a mixture of 1-2 (80 mg, 115.06 μmol, 1 eq) in DMF (1 mL) was added 1-1, EDCI (66.17 mg, 345.18 μmol, 3 eq), HOAt (15.66 mg, 115.06 μmol, 16.10 μL, 1 eq) and NMM (58.19 mg, 575.30 μmol, 63.25 μL, 5 eq) at 25° C., the mixture was stirred at 25° C. for 1 hr. The reaction mixture was diluted with H2O (1 mL) and extracted with DCM (1 mL×3), the combined organic layers were dried over Na₂SO₄, filtered and concentrated to give a residue. The residue was purified by column chromatography (SiO₂, EA/MeOH=1/0 to 6/4) and concentrated to give the product. 1-3 (45 mg, 41.76 μmol, 36.29% yield, 89.883% purity) was obtained as a yellow solid. LCMS: R^{t}=0.338 min, [M+H]⁺=968.5.

### Step 2: Synthesis of I-290

To a mixture of 1-3 (35 mg, 36.13 μmol, 1 eq) in DCM (0.5 mL) was added TFA (153.50 mg, 1.35 mmol, 0.1 mL, 37.26 eq) at 25° C., the mixture was stirred at 25° C. for 0.5 hr. The reaction mixture was concentrated to give a residue. The residue was purified by reversed phase column (0.1% TFA condition) and lyophilized to give the product. I-290 (8.69 mg, 9.76 μmol, 27.01% yield, 97.536% purity) was obtained as a white solid. LCMS: R^{t}=0.308 min, [M+H]⁺=868.4. ¹H NMR (400 MHZ, METHANOL-d4) δ=8.12 (s, 1H), 7.53-7.41 (m, 1H), 7.37-7.29 (m, 4H), 7.29-7.20 (m, 2H), 7.12 (d, J=3.6 Hz, 1H), 6.62 (d, J=3.6 Hz, 1H), 4.93-4.89 (m, 1H), 4.58 (d, J=4.8 Hz, 1H), 4.53-4.43 (m, 2H), 4.39 (d, J=12.0 Hz, 1H), 4.09-3.98 (m, 1H), 3.75 (s, 1H), 3.70-3.60 (m, 2H), 3.57-3.44 (m, 3H), 3.25-2.95 (m, 3H), 2.88-2.65 (m, 3H), 2.39-2.29 (m, 2H), 2.27-2.19 (m, 2H), 2.19-2.06 (m, 2H), 2.06-1.98 (m, 3H), 1.96 (d, J=7.6 Hz, 1H), 1.95-1.90 (m, 2H), 1.87 (d, J=9.2 Hz, 3H), 1.81-1.74 (m, 1H), 1.69-1.56 (m, 5H), 1.56-1.42 (m, 2H), 0.92-0.76 (m, 4H). SFC: R^{t}=1.828/2.184 min. ¹⁹F NMR (376 MHz, METHANOL-d4) δ=−123.74 (br s, 1F).

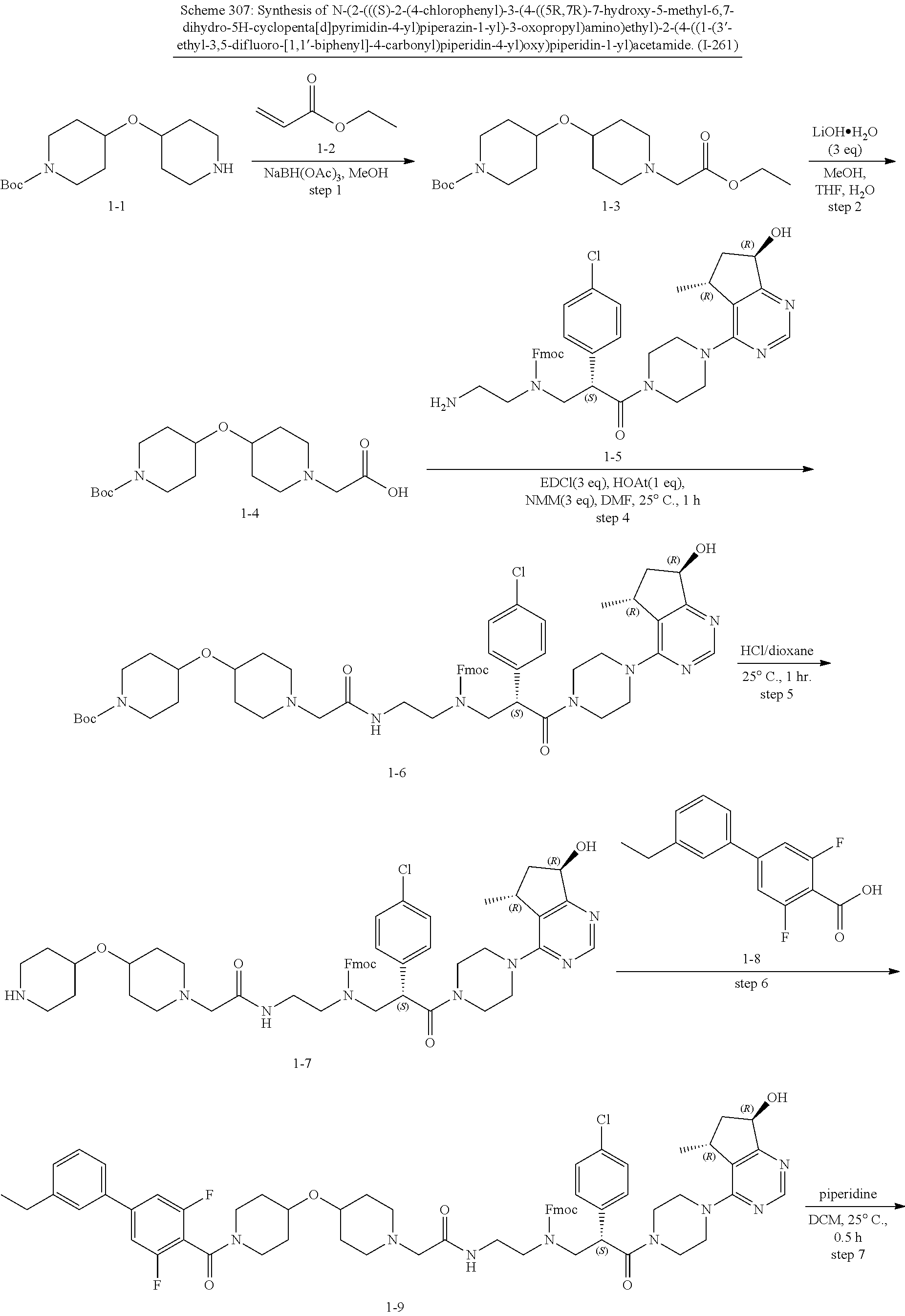

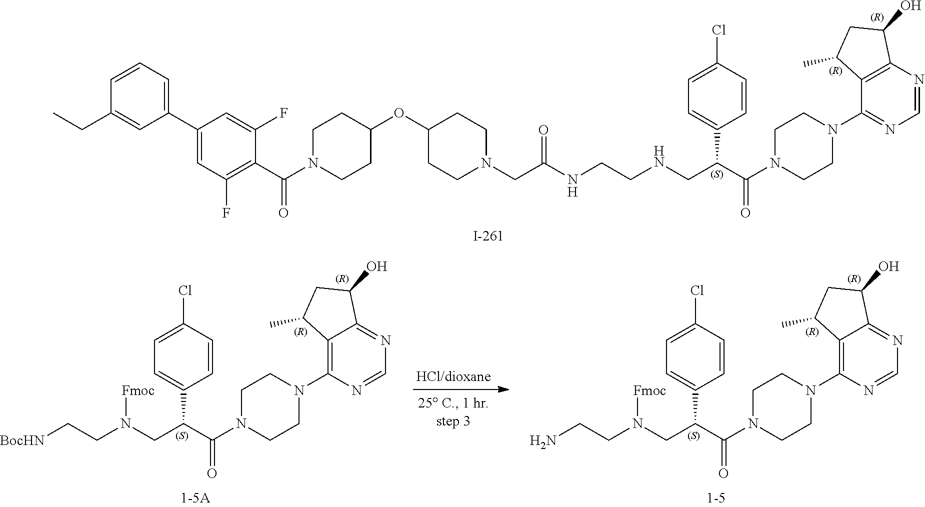

To a solution of 1-1 (4.5 g, 15.82 mmol, 1 eq) and 1-2 (4.85 g, 23.73 mmol, 1.5 eq) in DCM (50 mL) and the mixture was stirred at 25° C. for 0.5 h. Then NaBH(OAc)₃ (8.38 g, 39.56 mmol, 2.5 eq) was added to the mixture and the mixture was stirred at 25° C. for 1.5 h. The reaction mixture was quenched by addition H₂O 50 mL, and then diluted with DCM 50 mL and extracted with DCM 300 mL 100 mL*3). The combined organic layers were dried over anhydrous Na₂SO₄, filtered and concentrated under reduced pressure to give a residue. The residue was purified by flash silica gel chromatography (ISCO®; 120 g SepaFlash® Silica Flash Column, Eluent of 0˜100% Ethyl acetate: Methanol @ 80 mL/min). Concentrated under reduced pressure to give a product. 1-3 (4.5 g, 12.15 mmol, 76.76% yield) was obtained as yellow oil. ¹H NMR (400 MHZ, METHANOL-d4) δ=4.20-4.15 (m, 2H), 3.77-3.69 (m, 2H), 3.68-3.63 (m, 1H), 3.57-3.51 (m, 1H), 3.23 (s, 2H), 3.13 (s, 2H), 2.87-2.79 (m, 2H), 2.44-2.34 (m, 2H), 1.92-1.76 (m, 4H), 1.68-1.58 (m, 2H), 1.45 (s, 11H), 1.29-1.25 (m, 3H),

### Step 2: Synthesis of 1-4

To a solution of 1-3 (2 g, 5.40 mmol, 1 eq) in THF (8 mL), MeOH (8 mL) and H₂O (4 mL) was added LiOH·H₂O (679.60 mg, 16.20 mmol, 3 eq). The mixture was stirred at 25° C. for 1 hr. The reaction mixture was adjust pH=6 with aq. HCl (1 M), then diluted with EA 20 mL and extracted with EA 60 mL (20 mL*3). The combined organic layers were dried over anhydrous Na₂SO₄, filtered and concentrated under reduced pressure to give a residue. The residue was used in next step without further purification. 1-4 (1.8 g, 5.26 mmol, 97.37% yield) was obtained as a white solid. LCMS: [M+H]⁺=343.3.

### Step 3: Synthesis of 1-5

To a solution of 1-5A (1.5 g, 1.92 mmol, 1 eq) in DCM (15 mL) was added HCl/dioxane (2 M, 15.00 mL, 15.63 eq). The mixture was stirred at 25° C. for 1 hr. The reaction mixture was concentrated under reduced pressure to give a residue. The residue was used in next step without further purification. 1-5 (1.5 g, crude, HCl) was obtained as a white solid. LCMS: R^{t}=0.460 min, [M+H]⁺=681.3. SFC: R^{t}=0.971 min, ee value=100%.

### Step 4: Synthesis of 1-6

To a solution of 1-4 (550 mg, 1.61 mmol, 1 eq) and 1-5 (1.15 g, 1.61 mmol, 1 eq, HCl) in DMF (10 mL) was added EDCI (923.71 mg, 4.82 mmol, 3 eq), HOAt (218.62 mg, 1.61 mmol, 224.68 μL, 1 eq) and NMM (487.38 mg, 4.82 mmol, 529.76 μL, 3 eq). The mixture was stirred at 25° C. for 1 hr. The reaction mixture was quenched by addition H₂O 10 mL, and then diluted with EA 10 mL and extracted with EA 60 mL (20 mL*3). The combined organic layers were washed with brine 6 mL (10 mL*3), dried over anhydrous Na₂SO₄, filtered and concentrated under reduced pressure to give a residue. The residue was purified by flash silica gel chromatography (ISCO®; 40 g SepaFlash® Silica Flash Column, Eluent of 0˜100% Ethyl acetate/Petroleum ether gradient (@ XX mL/min). Concentrated under reduced pressure to give product. 1-6 (1.5 g. 1.37 mmol, 85.44% yield, 92% purity) was obtained as a yellow solid. LCMS: R^{t}=0.505 min, [M+H]⁺=1005.5. SFC: R^{t}=2.657 min.

### Step 5: Synthesis of 1-7

To a solution of 1-6 (500 mg, 497.20 μmol, 1 eq) in DCM (5 mL) was added HCl/dioxane (2 M, 5 mL, 20.11 eq). The mixture was stirred at 25° C. for 1 hr. The reaction mixture was concentrated under reduced pressure to give a residue. The residue was used in next step without further purification. 1-7 (450 mg, 477.72 μmol, 96.08% yield, HCl) was obtained as a yellow solid. LCMS: Rt: 0.427 min, [M+H]⁺=905.5.

### Step 6: Synthesis of 1-9

To a solution of 1-7 (350 mg, 371.56 μmol, 1 eq, HCl) and 1-8 (107.19 mg, 408.71 μmol, 1.1 eq) in DMF (3 mL) was added EDCI (213.68 mg, 1.11 mmol, 3 eq), HOAt (50.57 mg, 371.56 μmol, 51.98 μL, 1 eq) and NMM (112.75 mg, 1.11 mmol, 122.55 μL, 3 eq). The mixture was stirred at 25° C. for 1 hr. The reaction mixture was quenched by addition H₂O 3 mL, and then diluted with EA 3 mL and extracted with EA 9 mL (3 mL*3). The combined organic layers were washed with brine 9 mL (3 mL*3), dried over anhydrous Na₂SO₄, filtered and concentrated under reduced pressure to give a residue. The residue was purified by reversed phase (26.8*125 mm, of XB—C18, 20-40 μm, 80 Å) Mobile phase: A for H₂O (0.1% FA v/v) and B for acetonitrile; Gradient: B 0%-100% in 20 min; Flow rate: 60 mL/min; Column temperature: R.T. Wavelength: 220 nm/254 nm), then concentrated to remove organic solvents and lyophilized. 1-9 (330 mg, 278.41 μmol, 74.93% yield, 97% purity) was obtained as a white solid. LCMS: R^{t}=0.542 min, [M+H]⁺=1149.6. SFC: R^{t}=2.205 min, ee value=100%.

### Step 7: Synthesis of I-261

To a solution of 1-9 (300 mg, 260.93 μmol, 1 eq) in DCM (3 mL) was added piperidine (258.66 mg, 3.04 mmol, 300.00 μL, 11.64 eq). The mixture was stirred at 25° C. for 1 hr. The reaction mixture was quenched by addition H₂O 3 mL, and then diluted with DCM 3 mL and extracted with DCM 9 mL (3 mL*3). The combined organic layers were dried over anhydrous Na₂SO₄, filtered and concentrated under reduced pressure to give a residue. The residue was purified by prep-HPLC (column: Phenomenex luna C18 150*40 mm*15 um; mobile phase: [water (FA)-ACN]; gradient: 10%-40% B over 15 min), then concentrated to remove organic solvents and lyophilized to give product. The product was purified by prep-HPLC (column: Unisil 3-100 C18 Ultra 150*50 mm*3 um; mobile phase: [water (FA)-ACN]; gradient: 10%-40% B over 10 min), then concentrated to remove organic solvents and lyophilized to give product. I-261 (200 mg, 211.32 μmol, 80.99% yield, 98% purity) was obtained as a white solid. LCMS: R^{t}=1.104 min, [M+H]⁺=927.4. ¹H NMR (400 MHZ, METHANOL-d4) δ=8.44-8.25 (m, 2H), 7.50 (s, 1H), 7.49-7.43 (m, 3H), 7.42-7.33 (m, 5H), 7.29 (d, J=7.6 Hz, 1H), 4.99-4.96 (m, 1H), 4.49-4.43 (m, 1H), 4.12-4.03 (m, 1H), 3.94-3.90 (m, 1H), 3.84-3.80 (m, 2H), 3.76-3.70 (m, 1H), 3.68-3.46 (m, 10H), 3.45-3.38 (m, 1H), 3.29-3.20 (m, 4H), 3.18-3.13 (m, 2H), 2.96 (d, J=4.4 Hz, 2H), 2.76-2.70 (m, 2H), 2.59 (s, 2H), 2.17-2.02 (m, 2H), 2.01-1.91 (m, 3H), 1.90-1.86 (m, 1H), 1.82-1.55 (m, 5H), 1.30-1.26 (m, 3H), 1.10 (d, J=6.8 Hz, 3H). ¹⁹F NMR (377 MHz, METHANOL-d4) δ=−76.86 (br s, 1F), −115.40 (d, J=18.0 Hz, 2F). SFC: R^{t}=1.689 min, ee value=100%.

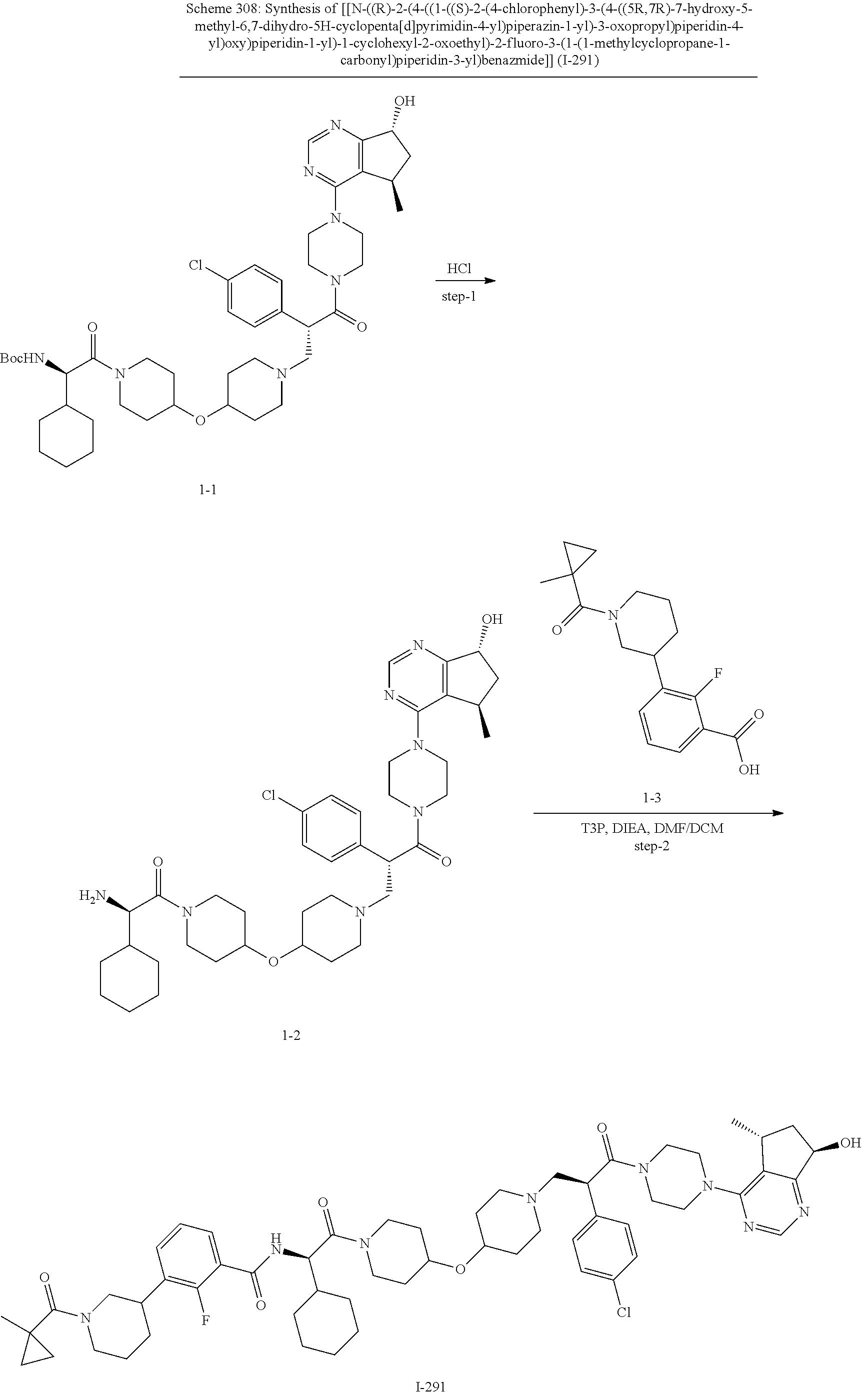 [SMILES: C[C@@H]1C[C@@H](O)c2ncnc(N3CCN(C(=O)[C@H](CN4CCC(OC5CCN(C(=O)[C@H](NC(=O)OC(C)(C)C)C6CCCCC6)CC5)CC4)c4ccc(Cl)cc4)CC3)c21 ; Cl ; C[C@@H]1C[C@@H](O)c2ncnc(N3CCN(C(=O)[C@H](CN4CCC(OC5CCN(C(=O)[C@H](N)C6CCCCC6)CC5)CC4)c4ccc(Cl)cc4)CC3)c21 ; CC1(C(=O)N2CCCC(c3cccc(C(=O)O)c3F)C2)CC1 ; C[C@@H]1C[C@@H](O)c2ncnc(N3CCN(C(=O)[C@H](CN4CCC(OC5CCN(C(=O)[C@H](NC(=O)c6cccc(C7CCCN(C(=O)C8(C)CC8)C7)c6F)C6CCCCC6)CC5)CC4)c4ccc(Cl)cc4)CC3)c21]

### Step 1: Synthesis of 1-2

The mixture of 1-1 (200 mg, 243.17 μmol, 1 eq) in DCM (2 mL) and HCl/dioxane (2 mL) was stirred at 25° C. for 1 hr. The reaction was concentrated under reduced pressure to give a residue. The crude product 1-2 (200 mg, crude, HCl salt) was a yellow solid. LCMS: Retention time: 0.757 min, [M+H]⁺=722.4.

### Step 2: Synthesis of I-291

To a solution of 1-2 (200 mg, 263.57 μmol, 1 eq, HCl salt) and 1-3 (96.57 mg, 316.28 μmol, 1.2 eq) in DMF (2 mL) and DCM (2 mL) was added T3P (569.72 mg, 790.70 μmol, 50% purity, 3 eq, CAS: 163755-62-2) and DIEA (340.64 mg, 2.64 mmol, 459.09 μL, 10 eq). The mixture was stirred at 25° C. for 1 hr. The reaction was cooled to room temperature, which was diluted with H₂O (5 mL), extracted with DCM 15 mL (5 mL*3), the organic phase was washed with saturated aqueous NaCl (5 mL). Then dried over Na₂SO₄, filtered and concentrated under reduced pressure to give a residue. The residue was purified by prep-HPLC (column: Waters Xbridge 150*25 mm*5 um; mobile phase: [water (NH4HCO3)-ACN]; gradient: 45%-75% B over 10 min). The eluent was concentrated under reduced pressure to remove ACN, then lyophilized to afford product. The residue was further purified by prep-HPLC (column: Phenomenex luna C18 150*25 mm*10 um; mobile phase: [water (FA)-ACN]; gradient: 23%-53% B over 11 min). The eluent was concentrated under reduced pressure to remove ACN, then lyophilized to afford product. I-291 (80 mg, 75.56 μmol, 28.67% yield, 99.71% purity, FA salt) was obtained as a white solid. LCMS: Retention time: 14.660 min, 14.839 min [M+H]⁺=1009.4. SFC: R^{t}=1.391 min, 1.821 min. ¹H NMR (400 MHZ, METHANOL-d4) δ=8.52 (br s, 1H), 8.43 (d, J=1.2 Hz, 1H), 7.61-7.48 (m, 2H), 7.42-7.30 (m, 4H), 7.26 (t, J=8.0 Hz, 1H), 5.00-4.93 (m, 2H), 4.56-4.44 (m, 2H), 4.38-4.25 (m, 1H), 4.05-4.00 (m, 1H), 3.95-3.74 (m, 5H), 3.72-3.51 (m, 7H), 3.49-3.34 (m, 3H), 3.27-3.16 (m, 2H), 3.11-3.03 (m, 1H), 2.99-2.86 (m, 2H), 2.64-2.34 (m, 3H), 2.18-1.99 (m, 3H), 1.97-1.73 (m, 10H), 1.73-1.57 (m, 6H), 1.56-1.44 (m, 1H), 1.40-1.20 (m, 6H), 1.20-1.03 (m, 5H), 1.02-0.82 (m, 2H), 0.72-0.52 (m, 2H). ¹⁹F NMR (377 MHz, METHANOL-d4) δ=−121.50.

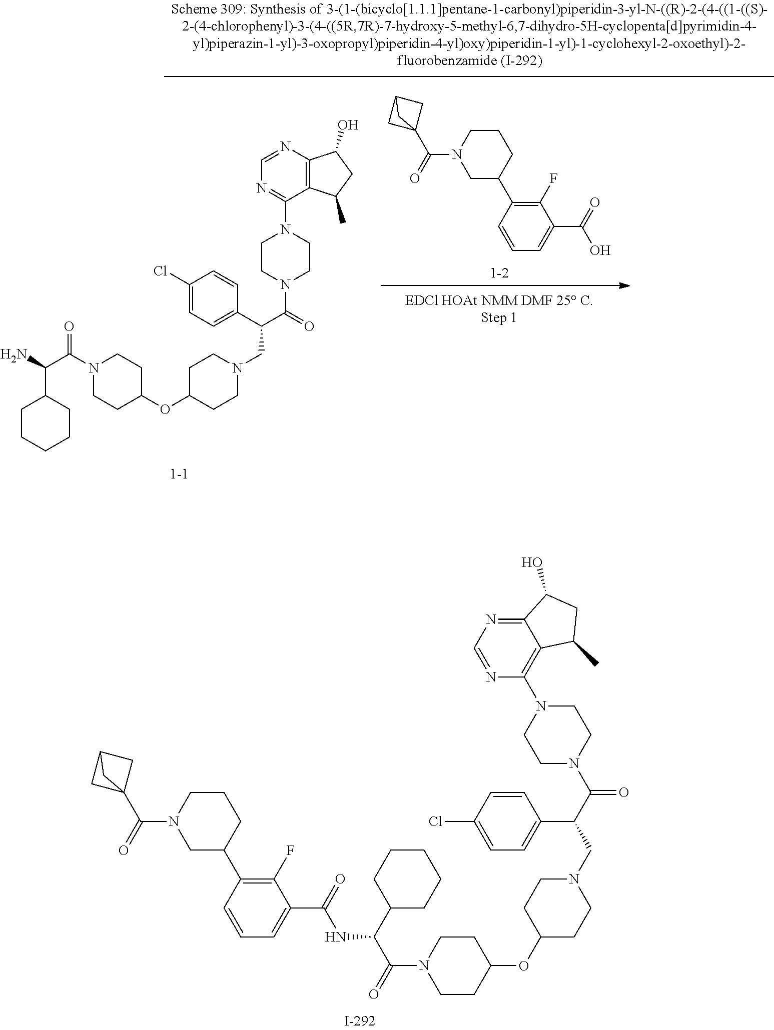

### Step 1: Synthesis of I-292

To a solution of 1-1 (150 mg, 197.68 μmol, 1 eq, HCl salt) and 1-2 (62.73 mg, 197.68 μmol, 1 eq) in DMF (2 mL) was added EDCI (113.68 mg, 593.03 μmol, 3 eq), HOAt (26.91 mg, 197.68 μmol, 27.65 μL, 1 eq) and NMM (159.95 mg, 1.58 mmol, 173.86 μL, 8 eq). The mixture was stirred at 25° C. for 1 hr. The reaction mixture was quenched by addition water 5 mL at 25° C., and then extracted with EA 15 mL (5 mL*3). The combined organic layers were dried over Na₂SO₄, filtered and concentrated under reduced pressure to give a residue. The residue was purified by prep-HPLC (column: Waters Xbridge 150*25 mm*5 um; mobile phase: [water (NH4HCO3)-ACN]; gradient: 48%-78% B over 10 min), and the target peak was concentrated and lyophilized to afford crude product. The product was further purified by prep-HPLC (column: Phenomenex luna C18 150*25 mm*10 um; mobile phase: [water (FA)-ACN]; gradient: 18%-48% B over 9 min), and the target peak was concentrated and lyophilized to afford desired product. I-292 (77 mg, 72.12 μmol, 36.48% yield, 100% purity, FA salt) was obtained as white solid. LCMS: R^{t}=1.194 min, [M+H]⁺=1021.8. SFC: R^{t}=1.573 min. ¹H NMR (400 MHZ, METHANOL-d4) δ=8.53 (s, 1H), 8.43 (d, J=1.2 Hz, 1H), 7.61-7.44 (m, 2H), 7.41-7.31 (m, 4H), 7.30-7.21 (m, 1H), 5.01-4.93 (m, 2H), 4.53 (d, J=12.4 Hz, 1H), 4.34 (d, J=10.0 Hz, 2H), 4.02 (m, 1H), 3.94-3.74 (m, 5H), 3.69 (s, 2H), 3.66-3.50 (m, 5H), 3.49-3.37 (m, 2H), 3.27-3.07 (m, 3H), 3.03-2.92 (m, 2H), 2.82-2.42 (m, 5H), 2.26-2.16 (m, 6H), 2.14-1.99 (m, 3H), 1.96-1.74 (m, 10H), 1.73-1.57 (m, 6H), 1.55-1.45 (m, 1H), 1.38-1.20 (m, 3H), 1.15 (m, 5H). ¹⁹F NMR (377 MHz, METHANOL-d4) δ=−121.45.

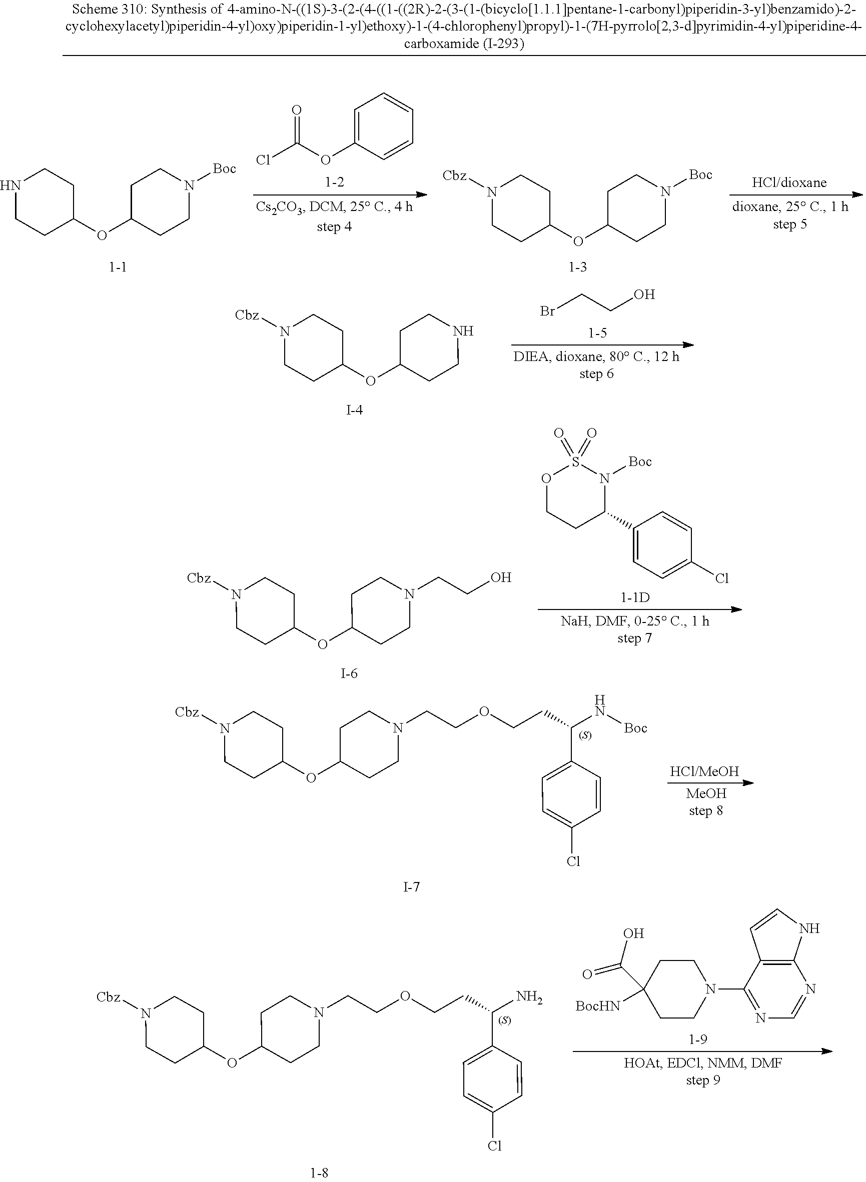

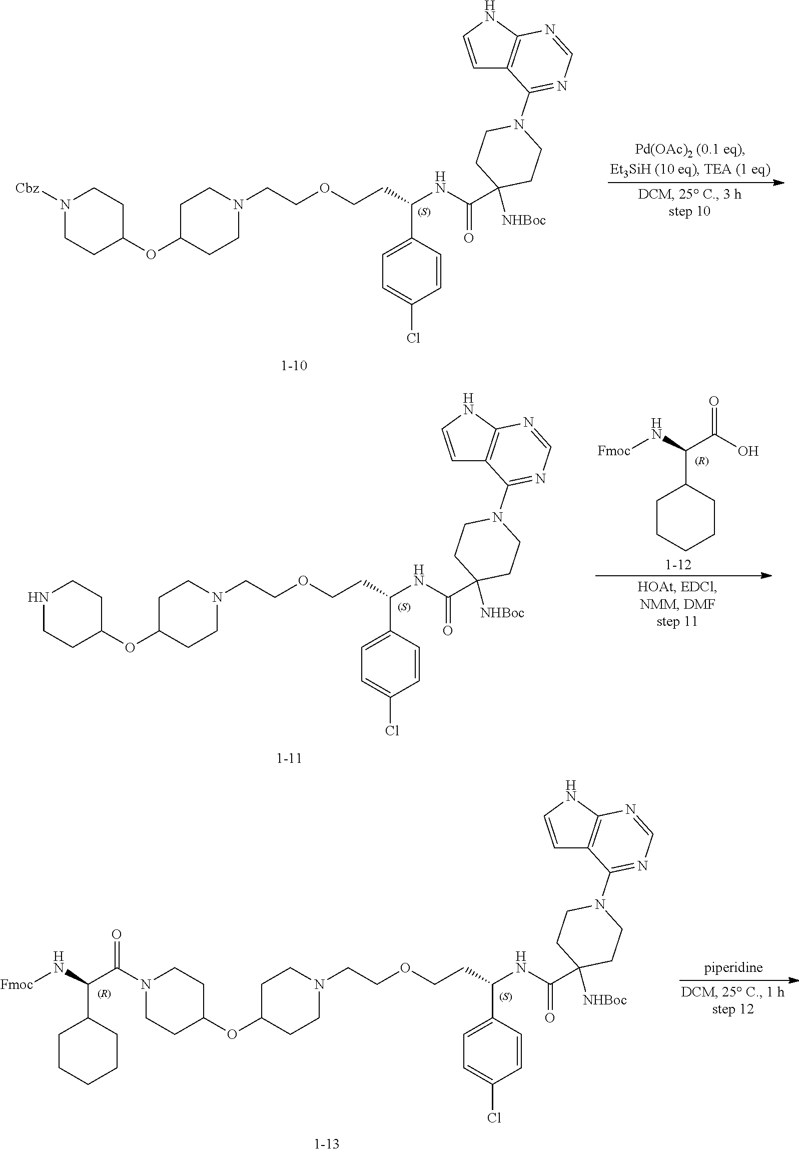

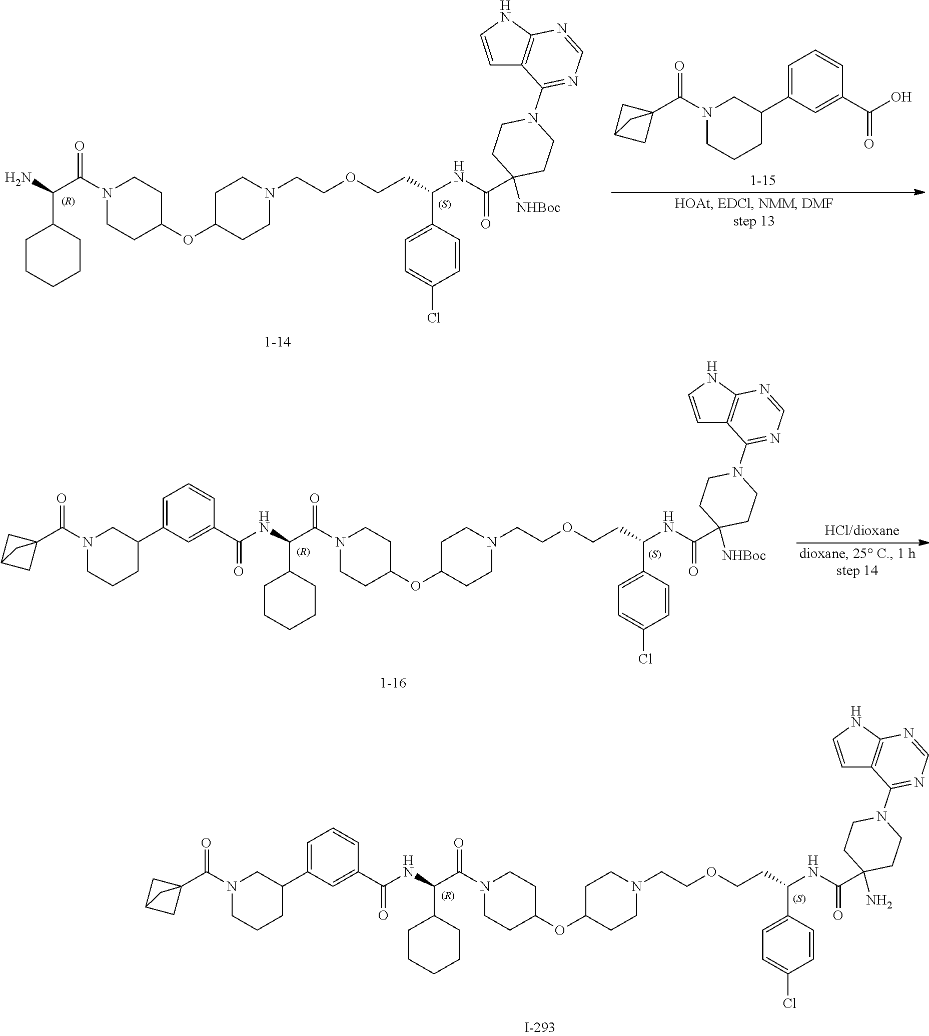

### Step 1: Synthesis of 1-1B

To a solution of 1-1A (15 g, 80.80 mmol, 1 eq) in DCM (150 mL) was added Boc2O (21.16 g, 96.96 mmol, 22.27 mL, 1.2 eq) and TEA (24.53 g, 242.39 mmol, 33.74 mL, 3 eq). The mixture was stirred at 25° C. for 1 hr. The reaction was diluted with H₂O (50 mL), extracted with DCM (100 mL*3), the organic phase was washed with saturated aqueous NaCl (50 mL). Then dried over [Na₂SO₄], filtered and concentrated under reduced pressure to give a residue. The crude product was purified by re-crystallization from PE (100 mL) at 20° C., then filtered and concentrated under reduced pressure to give 1-1B (19.5 g, 64.35 mmol, 79.64% yield, 94.3% purity) as white solid. LCMS: R^{t}=0.511 min, [M+Na]⁺=308.1.

### Step 2: Synthesis of 1-1C

To a solution of imidazole (18.58 g, 272.95 mmol, 4 eq) and TEA (15.19 g, 150.12 mmol, 20.90 mL, 2.2 eq) in DCM (150 mL) was cooled to −60° C., then added SOCl₂ (9.74 g, 81.89 mmol, 5.95 mL, 1.2 eq). The mixture was stirred at −60° C. for 0.5 hr. Then a solution of 1-1B (19.5 g, 68.24 mmol, 1 eq) in DCM (100 mL) was added. The reaction mixture was then stirred at 25° C. for 12 hr. The reaction mixture was poured into water (200 mL) and extracted with DCM (150 mL*2), the organic layer was washed with brine (100 mL) and dried over Na₂SO₄. Concentrated to get 1-1C (22 g, 66.30 mmol, 97.16% yield) as an off-white solid. HNMR Data (400 MHz, DMSO-d₆) δ=7.44 (d, J=8.4 Hz, 2H), 7.31 (d, J=8.4 Hz, 2H), 5.32-5.25 (m, 1H), 4.44-4.29 (m, 1H), 3.82 (m, 1H), 3.03-2.83 (m, 1H), 2.32 (m, 1H), 1.37 (s, 9H).

### Step 3: Synthesis of 1-1D

To a solution of 1-1C (22 g, 66.30 mmol, 1 eq) in MeCN (200 mL) was added RuCl₃ (1.38 g, 6.63 mmol, 442.22 μL, 0.1 eq), H₂O (200 mL) and NaIO₄ (15.60 g, 72.93 mmol, 4.04 mL, 1.1 eq) at 0° C. The mixture was stirred at 25° C. for 1 hr. The reaction was diluted with Sat. NaHCO₃ (100 mL), extracted with ethyl acetate (100 mL*3), the organic phase was washed with saturated aqueous NaCl (50 mL). Then dried over (Na₂SO₄), filtered and concentrated under reduced pressure to give a crude product. The aqueous phase was quenched by Sat. Na₂S203 (100 mL). The crude product was triturated with PE:EA=5:1 (100 mL) at 25° C. for 30 min to get 1-1D (18.255 g, 50.91 mmol, 76.79% yield, 97% purity) as an off-white solid. LCMS: R^{t}=0.554 min, [2M+Na]⁺=717.1. HNMR (400 MHZ, DMSO-d₆) δ=7.54-7.45 (m, 2H), 7.43-7.36 (m, 2H), 5.65 (m, 1H), 4.78-4.64 (m, 1H), 4.41 (m, 1H), 2.80-2.67 (m, 1H), 2.65-2.55 (m, 1H), 1.41 (s, 9H).

### Step 4: Synthesis of 1-3

To a solution of 1-1 (20 g, 70.33 mmol, 1 eq) in DCM (200 mL) was added Cs₂CO₃ (45.83 g, 140.65 mmol, 2 eq) and 1-2 (14.40 g, 84.39 mmol, 12.05 mL, 1.2 eq). The mixture was stirred at 25° C. for 4 hr. The reaction mixture was concentrated to get a residue. The residue was purified by flash silica gel chromatography (ISCO®; 220 g SepaFlash® Silica Flash Column, Eluent of 0˜60% Ethyl acetate/Petroleum ether gradient (a) 100 mL/min) and the eluent was concentrated to get 1-3 (9 g, 21.50 mmol, 30.58% yield) as colorless oil. ¹H NMR (400 MHZ, CHLOROFORM-d) δ=7.41-7.35 (m, 5H), 5.13 (s, 2H), 3.89-3.69 (m, 4H), 3.65-3.48 (m, 2H), 3.31-3.17 (m, 2H), 3.10 (m, 2H), 1.89-1.70 (m, 4H), 1.51 (m, 4H), 1.46 (s, 9H).

### Step 5: Synthesis of 1-4

To a solution of 1-3 (9 g, 21.50 mmol, 1 eq) in dioxane (50 mL) was added HCl/dioxane (4 M, 50 mL, 9.30 eq). The mixture was stirred at 25° C. for 1 hr. The reaction mixture was concentrated to get 1-4 (6.6 g, 18.60 mmol, 86.49% yield, HCl salt) as a white solid. ¹H NMR (400 MHZ, DMSO-d₆) δ=9.04 (br d, J=1.6 Hz, 2H), 7.44-7.25 (m, 5H), 5.06 (s, 2H), 3.80-3.56 (m, 4H), 3.11 (br s, 4H), 2.92 (br s, 2H), 2.00-1.83 (m, 2H), 1.82-1.72 (m, 2H), 1.71-1.58 (m, 2H), 1.42-1.27 (m, 2H).

### Step 6: Synthesis of 1-6

To a solution of 1-4 (2 g, 5.64 mmol, 1 eq, HCl) in dioxane (20 mL) was added DIEA (3.64 g, 28.18 mmol, 4.91 mL, 5 eq) and 1-5 (1.06 g, 8.45 mmol, 599.22 μL, 1.5 eq). The mixture was stirred at 80° C. for 12 hr. The mixture was concentrated to get a residue. The residue was purified by reversed phase HPLC (ISCO®; 80 g SepaFlash® C18 Column, Eluent of 0˜90% (0.1% NH₃·H₂O) water/MeCN @ 90 mL/min), the collected eluent was lyophilized to get 1-6 (1.7 g, 4.69 mmol, 83.22% yield, 100% purity) as yellow oil. LCMS: R^{t}=0.549 min, [M+H]⁺=363.2.

### Step 7: Synthesis of 1-7

To a solution of 1-6 (1 g, 2.76 mmol, 1 eq) in DMF (10 mL) was cooled to 0° C. under N₂ then added NaH (134.00 mg, 3.35 mmol, 60% purity, 1.21 eq) at 0° C. The mixture was stirred at 0° C. for 0.5 hr, then 1-1D (959.59 mg, 2.76 mmol, 1 eq) was added. The mixture was then warm to 25° C., and stirred for 1 hr. The reaction mixture was quenched by water (1 mL) to get a mixture. The mixture was purified by reversed phase (26.8*125 mm, 120 g of XB—C18, 20-40 μm, 120 Å) Mobile phase: A for H₂O (0.1% HCl v/v) and B for acetonitrile; Gradient: B 5%-75% in 30 min; Flow rate: 60 mL/min; Column temperature: R.T. Wavelength: 220 nm/254 nm), then the eluent was lyophilized to get 1-7 (500 mg, 767.20 μmol, 27.81% yield, 96.7% purity) as a white solid. LCMS: R^{t}=0.576 min, [M+H]⁺=630.4. ¹H NMR (400 MHZ, METHANOL-d4) δ=7.42 (br d, J=8.4 Hz, 2H), 7.37-7.33 (m, 4H), 7.33-7.26 (m, 3H), 5.71 (m, 1H), 5.11 (s, 2H), 3.92 (br s, 1H), 3.84-3.65 (m, 8H), 3.48 (m, 2H), 3.27-3.12 (m, 3H), 3.08-2.93 (m, 1H), 2.61-2.48 (m, 1H), 2.40-2.22 (m, 2H), 2.17-2.05 (m, 1H), 2.03-1.91 (m, 2H), 1.83 (br s, 2H), 1.52 (br d, J=8.0 Hz, 2H), 1.46-1.35 (m, 1H), 1.23 (s, 9H).

### Step 8: Synthesis of 1-8

To a solution of Intermediate 1-7 (500 mg, 793.38 μmol, 1 eq) in dioxane (2.5 mL) was added HCl/dioxane (4 M, 2.5 mL, 12.60 eq). The mixture was stirred at 25° C. for 0.5 hr. The reaction mixture was concentrated to get 1-8 (450 mg, 786.33 μmol, 99.11% yield, 99% purity, HCl salt) as a white solid. LCMS: R^{t}=0.417 min, [M+H]⁺=530.3. SFC: R^{t}=2.165 min.

### Step 9: Synthesis of 1-10

To a solution of 1-8 (450 mg, 794.27 μmol, 1 eq, HCl) and 1-9 (287.05 mg, 794.27 μmol, 1 eq) in DMF (5 mL) was added HOAt (162.16 mg, 1.19 mmol, 166.66 μL, 1.5 eq), EDCI (304.52 mg, 1.59 mmol, 2 eq) and NMM (401.69 mg, 3.97 mmol, 436.62 μL, 5 eq). The mixture was stirred at 25° C. for 1 hr. The reaction mixture was quenched by water (1 mL) to get a mixture. The mixture was purified by reversed phase HPLC (ISCO®; 80 g SepaFlash® C18 Column, Eluent of 0˜60% (0.1% FA) water/MeCN @ 60 mL/min). Collected eluent and lyophilized to get 1-10 (680 mg, 778.50 μmol, 98.01% yield, 100% purity) as a white solid. LCMS: R^{t}=0.477 min, [M+H]⁺=873.4. SFC: R^{t}=0.911 min.

### Step 10: Synthesis of 1-11

To a solution of 1-10 (680 mg, 778.5 μmol, 1 eq) in DCM (6.8 mL) was added Pd(OAc)₂ (17.48 mg, 77.84 μmol, 0.1 eq), TEA (118.16 mg, 1167.74 μmol, 162.54 μL, 1.5 eq) and Et₃SiH (905.22 mg, 7.78 mmol, 1243.42 μL, 10 eq) at 0° C. The mixture was stirred at 25° C. for 3 hr. The residue was purified by reversed phase HPLC (ISCO®; 40 g SepaFlash® C18 Column, Eluent of 0˜60% (0.1% FA) water/MeCN @60 mL/min). Collected the eluent and lyophilized to get a solid. Then the solid was dissolved in 10% of NaHCO₃ aqueous solution (10 mL), and extracted with (CHCl₃: i-PrOH=3:1) (10 mL*10), collected the organic phase, dried over Na₂SO₄ and concentrated to get 1-11 (280 mg, 256.56 μmol, 45.80% yield, 100% purity) as a white solid. LCMS: R^{t}=0.395 min, [M+H]⁺=739.5.

### Step 11: Synthesis of 1-13

To a solution of 1-11 (280 mg, 378.71 μmol, 1 eq) and 1-12 (143.70 mg, 378.71 μmol, 143.70 μL, 1 eq) in DMF (2.8 mL) was added HOAt (77.32 mg, 568.07 μmol, 79.47 μL, 1.5 eq), EDCI (145.20 mg, 757.43 μmol, 2 eq) and NMM (229.83 mg, 2.27 mmol, 249.82 μL, 6 eq). The mixture was stirred at 25° C. for 1 hr. The reaction was quenched by water (0.5 mL) to get a mixture. The mixture was purified by reversed phase HPLC (ISCO®; 45 g SepaFlash® C18 Column, Eluent of 0˜95% (0.1% FA) water/MeCN @ 30 mL/min). Collected the eluent and concentrated to get 1-13 (210 mg, 186.96 μmol, 49.37% yield, 98% purity) as a white solid. LCMS: R^{t}=0.568 min, [M+H]⁺=1100.7. SFC: R^{t}=1.776 min. ¹H NMR (400 MHz, CHLOROFORM-d) δ=9.60 (br d, J=12.8 Hz, 1H), 8.30 (d, J=1.8 Hz, 1H), 7.92-7.82 (m, 1H), 7.77 (d, J=7.6 Hz, 2H), 7.66-7.56 (m, 2H), 7.45-7.36 (m, 2H), 7.35-7.28 (m, 3H), 7.25-7.19 (m, 2H), 7.08-7.01 (m, 1H), 6.55-6.46 (m, 1H), 5.73 (m, 1H), 5.12 (br d, J=5.2 Hz, 1H), 4.55 (br dd, J=6.4, 9.1 Hz, 1H), 4.46-4.28 (m, 4H), 4.26-4.14 (m, 1H), 3.99-3.81 (m, 1H), 3.80-3.49 (m, 7H), 3.48-3.21 (m, 4H), 2.98-2.67 (m, 4H), 2.56-2.43 (m, 1H), 2.36-2.22 (m, 2H), 2.20-2.08 (m, 4H), 1.99-1.88 (m, 4H), 1.83-1.54 (m, 11H), 1.46 (s, 9H), 1.30-0.97 (m, 5H).

### Step 12: Synthesis of 1-14

To a solution of 1-13 (210 mg, 190.77 μmol, 1 eq) in DMF (2 mL) was added piperidine (48.73 mg, 572.32 μmol, 56.52 μL, 3 eq). The mixture was stirred at 25° C. for 1 hr. The reaction mixture was quenched by water (0.5 mL) to get a mixture. The mixture was purified by reversed phase HPLC (ISCO®; 30 g SepaFlash® C18 Column, Eluent of 0˜95% (0.1% FA) water/MeCN @ 20 mL/min). Collected the eluent and lyophilized to get a solid. Then the solid was dissolved into 10% of NaHCO₃ aqueous solution (5 mL), extracted with (CHCl₃: i-PrOH=3:1) (5 mL*10), collected the organic phase, dried over Na₂SO₄ and concentrated to get 1-14 (155 mg, 144.67 μmol, 75.83% yield, 82% purity) as a white solid. LCMS: R^{t}=0.419 min, [M+H]⁺=878.4.

### Step 13: Synthesis of 1-16

To a solution of 1-14 (50 mg, 56.91 μmol, 1 eq) and 1-15 (17.04 mg, 56.91 μmol, 1 eq) in DMF (0.5 mL) was added HOAt (11.62 mg, 85.37 μmol, 11.94 μL, 1.5 eq), EDCI (21.82 mg, 113.83 μmol, 2 eq) and NMM (28.78 mg, 284.56 μmol, 31.29 μL, 5 eq). The mixture was stirred at 25° C. for 1 hr. The reaction mixture was quenched by water (0.5 mL) to get a mixture. The mixture was purified by reversed phase HPLC (ISCO®; 30 g SepaFlash® C18 Column, Eluent of 0˜50% (0.1% FA) water/MeCN @ 20 mL/min). Collected the eluent and lyophilized to get 1-16 (45 mg, 38.02 μmol, 66.81% yield, 98% purity) as a white solid. LCMS: R^{t}=0.528 min, [M+H]⁺=1159.6. SFC: R^{t}=2.311 min & 2.772 min. ¹H NMR (400 MHZ, CHLOROFORM-d) δ=9.64-9.31 (m, 1H), 8.48 (s, 1H), 8.29 (d, J=6.0 Hz, 1H), 7.96-7.78 (m, 1H), 7.75-7.59 (m, 2H), 7.44-7.33 (m, 2H), 7.29 (br d, J=2.8 Hz, 1H), 7.25-7.19 (m, 2H), 7.13-6.95 (m, 2H), 6.52 (br s, 1H), 5.20-5.01 (m, 2H), 4.73-4.57 (m, 1H), 4.45-4.31 (m, 2H), 4.30-4.20 (m, 1H), 3.97-3.76 (m, 2H), 3.75-3.63 (m, 3H), 3.62-3.34 (m, 7H), 3.10-2.97 (m, 1H), 2.96-2.83 (m, 2H), 2.82-2.66 (m, 3H), 2.64-2.51 (m, 2H), 2.48-2.42 (m, 1H), 2.38-2.24 (m, 2H), 2.23-2.08 (m, 9H), 2.08-1.93 (m, 5H), 1.82-1.71 (m, 9H), 1.70-1.51 (m, 6H), 1.47 (s, 9H), 1.29-1.06 (m, 5H).

### Step 14: Synthesis of I-293

To a solution of 1-16 (45 mg, 38.80 μmol, 1 eq) in dioxane (1 mL) was added HCl/dioxane (4 M, 1 mL, 59.96 eq). The mixture was stirred at 25° C. for 0.5 hr. The reaction mixture was concentrated and quenched by NaHCO₃ (1 mL) and MeOH (2 mL) to get a mixture. The mixture was purified by reversed phase HPLC (ISCO®; 30 g SepaFlash® C18 Column, Eluent of 0˜40% (0.1% FA) water/MeCN @ 20 mL/min). Collected the eluent and lyophilized to I-293 (35 mg, 31.65 μmol, 81.58% yield, 100% purity, FA) as a white solid. LCMS: R^{t}=0.484 min, [M+H]⁺=1059.7. SFC: R^{t}=3.166 min, 3.996 min. ¹H NMR (400 MHZ, METHANOL-d4) δ=8.52 (s, 1H), 8.14 (s, 1H), 7.80-7.65 (m, 2H), 7.52-7.39 (m, 2H), 7.34 (d, J=2.8 Hz, 4H), 7.13 (m, 1H), 6.63 (m, 1H), 5.20-5.07 (m, 1H), 4.98-4.90 (m, 1H), 4.60-4.46 (m, 3H), 4.32 (br s, 1H), 4.08-3.97 (m, 1H), 3.96-3.78 (m, 1H), 3.72 (br s, 3H), 3.68-3.57 (m, 4H), 3.56-3.44 (m, 3H), 3.28-3.21 (m, 2H), 3.21-3.11 (m, 3H), 3.10-2.95 (m, 2H), 2.82-2.61 (m, 2H), 2.53-2.41 (m, 1H), 2.26-2.09 (m, 9H), 2.08-1.98 (m, 3H), 1.97-1.75 (m, 10H), 1.74-1.41 (m, 8H), 1.40-1.18 (m, 3H), 1.18-0.99 (m, 2H),

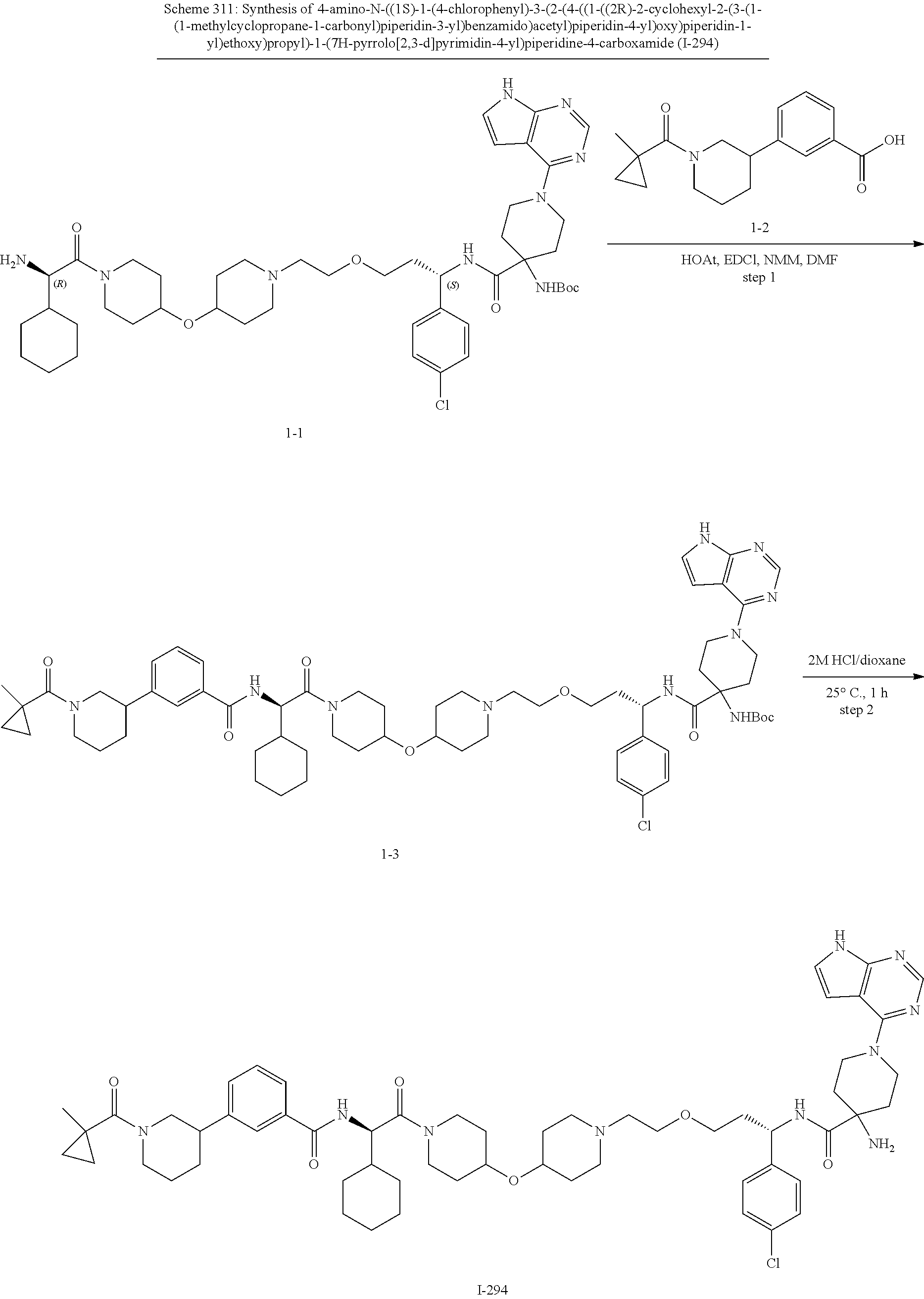

To a solution of 1-1 (50 mg, 56.91 μmol, 1 eq) and 2 (16.35 mg, 56.91 μmol, 1 eq) in DMF (0.5 mL) was added HOAt (11.62 mg, 85.37 μmol, 11.94 μL, 1.5 eq), EDCI (21.82 mg, 113.83 μmol, 2 eq) and NMM (28.78 mg, 284.56 μmol, 31.29 μL, 5 eq). The mixture was stirred at 25° C. for 1 hr. The reaction mixture was quenched by water (0.5 mL) to get a mixture. The mixture was purified by reversed phase HPLC (ISCO®; 30 g SepaFlash® C18 Column, Eluent of 0˜50% (0.1% FA) water/MeCN @ 20 mL/min). Collected the eluent and lyophilized to get 1-3 (50 mg, 42.69 μmol, 75.01% yield, 98% purity) as a white solid. LCMS: R^{t}=0.535 min, [M+H]⁺=1147.9. SFC: R^{t}=2.237 min & 2.720 min. ¹H NMR (400 MHZ, CHLOROFORM-d) δ=9.69-9.37 (m, 1H), 8.49 (s, 1H), 8.29 (d, J=5.6 Hz, 1H), 7.94-7.79 (m, 1H), 7.73 (s, 1H), 7.70-7.63 (m, 1H), 7.44-7.35 (m, 2H), 7.29 (br d, J=2.8 Hz, 1H), 7.25-7.20 (m, 2H), 7.13-7.01 (m, 2H), 6.52 (m, 1H), 5.56-5.25 (m, 1H), 5.19-5.02 (m, 2H), 4.64-4.44 (m, 2H), 4.43-4.30 (m, 2H), 4.00-3.76 (m, 2H), 3.73-3.52 (m, 6H), 3.51-3.36 (m, 4H), 3.02-2.66 (m, 7H), 2.63-2.41 (m, 2H), 2.38-2.02 (m, 7H), 2.01-1.90 (m, 4H), 1.73-1.52 (m, 8H), 1.47 (s, 9H), 1.32 (s, 3H), 1.30-1.05 (m, 6H), 1.02-0.91 (m, 2H), 0.62-0.53 (m, 2H).

### Step 2: Synthesis of I-294

To a solution of 1-3 (50 mg, 43.56 μmol, 1 eq) in dioxane (1 mL) was added HCl/dioxane (4 M, 1 mL, 59.96 eq). The mixture was stirred at 25° C. for 0.5 hr. The reaction mixture was concentrated and quenched by NaHCO₃ (1 mL) and MeOH (2 mL) to get a mixture. The mixture was purified by reversed phase HPLC (ISCO®; 30 g SepaFlash® C18 Column, Eluent of 0˜40% (0.1% FA) water/MeCN @ 20 mL/min). Collected the eluent and lyophilized to I-294 (50 mg, 42.16 μmol, 96.80% yield, 100% purity, FA salt) as a white solid. LCMS: R^{t}=0.479 min, [M+H]⁺=1047.7. SFC: R^{t}=2.548 min, 3.082 min. 1H NMR (400 MHZ, METHANOL-d4) δ=8.54 (s, 2H), 8.13 (d, J=1.6 Hz, 1H), 7.76 (s, 1H), 7.71 (d, J=7.6 Hz, 1H), 7.53-7.40 (m, 2H), 7.33 (d, J=2.0 Hz, 4H), 7.13 (m, 1H), 6.62 (m, 1H), 5.15-5.04 (m, 1H), 4.97-4.90 (m, 1H), 4.58-4.41 (m, 4H), 4.08-3.97 (m, 1H), 3.81 (br s, 1H), 3.76-3.55 (m, 7H), 3.54-3.42 (m, 3H), 3.29-3.21 (m, 1H), 3.13 (m, 3H), 3.02 (br s, 2H), 2.95-2.66 (m, 4H), 2.24-2.00 (m, 5H), 1.99-1.84 (m, 7H), 1.83-1.74 (m, 4H), 1.73-1.41 (m, 8H), 1.37-1.20 (m, 6H), 1.18-1.03 (m, 2H), 0.99-0.82 (m, 2H), 0.62 (br s, 2H).

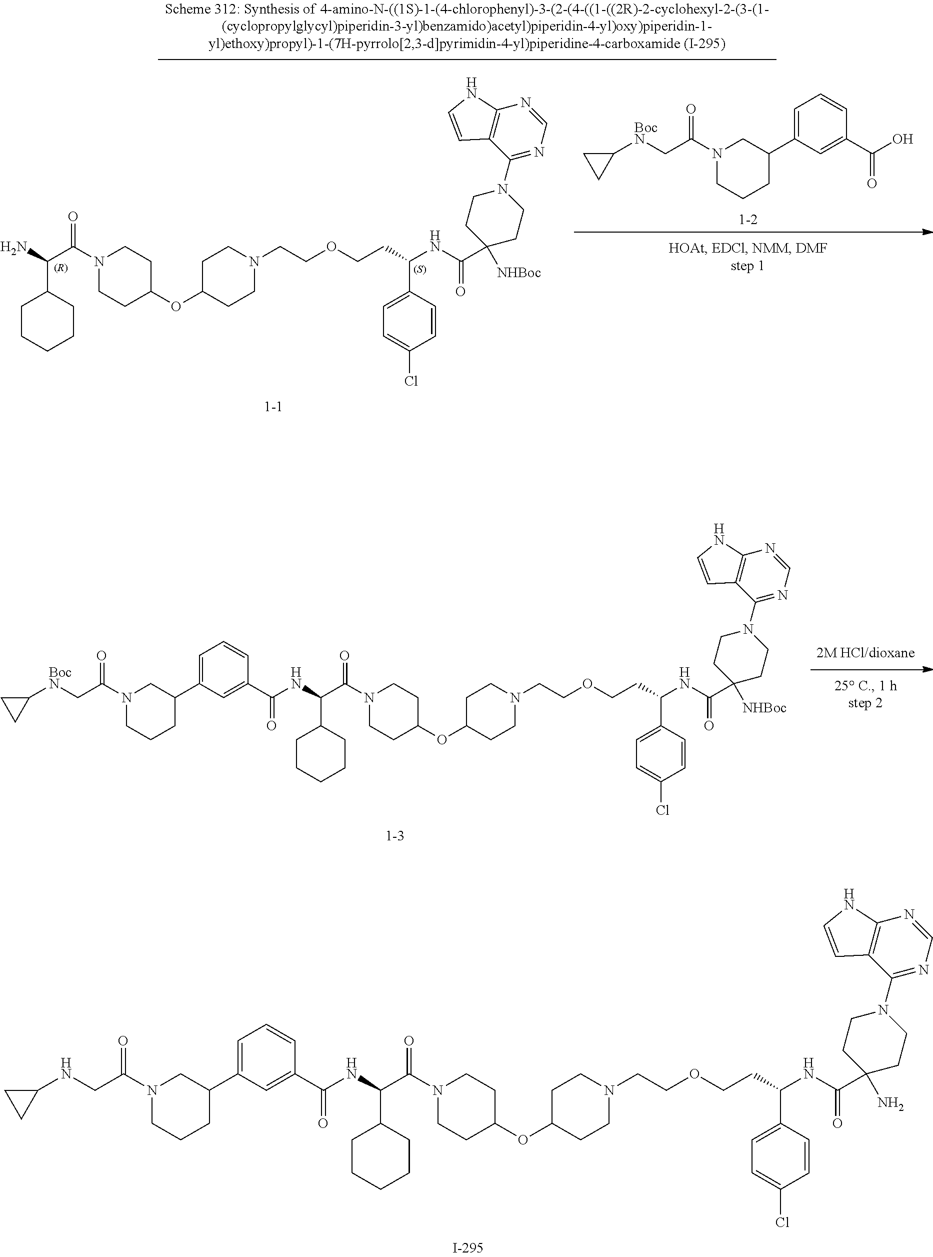

### Step 1: Synthesis of 3

To a solution of 1 (50 mg, 56.91 μmol, 1 eq) and 2 (22.91 mg, 56.91 μmol, 1 eq) in DMF (0.5 mL) was added HOAt (11.62 mg, 85.37 μmol, 11.94 μL, 1.5 eq), EDCI (21.82 mg, 113.83 μmol, 2 eq) and NMM (28.78 mg, 284.56 μmol, 31.29 μL, 5 eq). The mixture was stirred at 25° C. for 1 hr. The reaction mixture was quenched by water (0.5 mL) to get a mixture. The mixture was purified by reversed phase HPLC (ISCO®; 30 g SepaFlash® C18 Column, Eluent of 0˜50% (0.1% FA) water/MeCN @ 20 mL/min). Collected the eluent and lyophilized to get 3 (47 mg, 37.21 μmol, 65.39% yield) as a white solid. LCMS: R^{t}=0.556 min, [M+H]⁺=1263.0. SFC: R^{t}=0.911 min & 1.102 min. ¹H NMR (400 MHZ, CHLOROFORM-d) 8=9.87-9.49 (m, 1H), 8.51 (s, 1H), 8.28 (d, J=7.2 Hz, 1H), 7.85 (m, 1H), 7.76-7.60 (m, 2H), 7.43-7.32 (m, 2H), 7.28 (br d, J=2.0 Hz, 1H), 7.24-7.19 (m, 2H), 7.15-7.00 (m, 2H), 6.55-6.47 (m, 1H), 5.60-5.22 (m, 1H), 5.19-4.95 (m, 2H), 4.80-4.57 (m, 1H), 4.51-4.25 (m, 2H), 4.14-4.02 (m, 1H), 4.00-3.74 (m, 4H), 3.73-3.62 (m, 3H), 3.61-3.50 (m, 3H), 3.49-3.32 (m, 4H), 3.15-2.98 (m, 1H), 2.97-2.83 (m, 2H), 2.82-2.68 (m, 4H), 2.67-2.40 (m, 3H), 2.38-2.23 (m, 2H), 2.23-2.03 (m, 6H), 1.87-1.54 (m, 17H), 1.46 (s, 18H), 1.28-1.07 (m, 5H), 0.80-0.50 (m, 4H).

### Step 2: Synthesis of I-295

To a solution of 3 (47 mg, 37.21 μmol, 1 eq) in dioxane (1 mL) was added HCl/dioxane (4 M, 1 mL, 59.96 eq). The mixture was stirred at 25° C. for 0.5 hr. The reaction mixture was concentrated and quenched by NaHCO₃ (1 mL) and MeOH (2 mL) to get a mixture. The mixture was purified by reversed phase HPLC (ISCO®; 30 g SepaFlash® C18 Column, Eluent of 0˜40% (0.1% FA) water/MeCN @ 20 mL/min). Collected the eluent and lyophilized to I-295 (39 mg, 32.48 μmol, 87.27% yield, 100% purity, FA salt) as a white solid. LCMS: R^{t}=0.426 min, [M+H]⁺=1062.8. SFC: R^{t}=7.199 min, 8.004 min. 1H NMR (400 MHZ, METHANOL-d4) δ=8.58-8.50 (m, 5H), 8.13 (d, J=1.2 Hz, 1H), 7.77 (s, 1H), 7.74-7.68 (m, 1H), 7.53-7.38 (m, 2H), 7.32 (d, J=2.4 Hz, 4H), 7.12 (m, 1H), 6.62 (m, 1H), 5.11-5.02 (m, 1H), 4.96-4.91 (m, 1H), 4.63-4.43 (m, 3H), 4.08-3.98 (m, 1H), 3.94-3.85 (m, 1H), 3.85-3.68 (m, 2H), 3.68-3.53 (m, 7H), 3.51-3.42 (m, 3H), 3.28-3.07 (m, 2H), 3.00 (br s, 2H), 2.85 (br s, 2H), 2.82-2.67 (m, 2H), 2.67-2.53 (m, 2H), 2.24 (m, 1H), 2.21-2.11 (m, 2H), 2.11-1.99 (m, 3H), 1.98-1.83 (m, 7H), 1.82-1.48 (m, 12H), 1.37-1.19 (m, 3H), 1.18-1.02 (m, 2H), 0.54-0.31 (m, 4H).

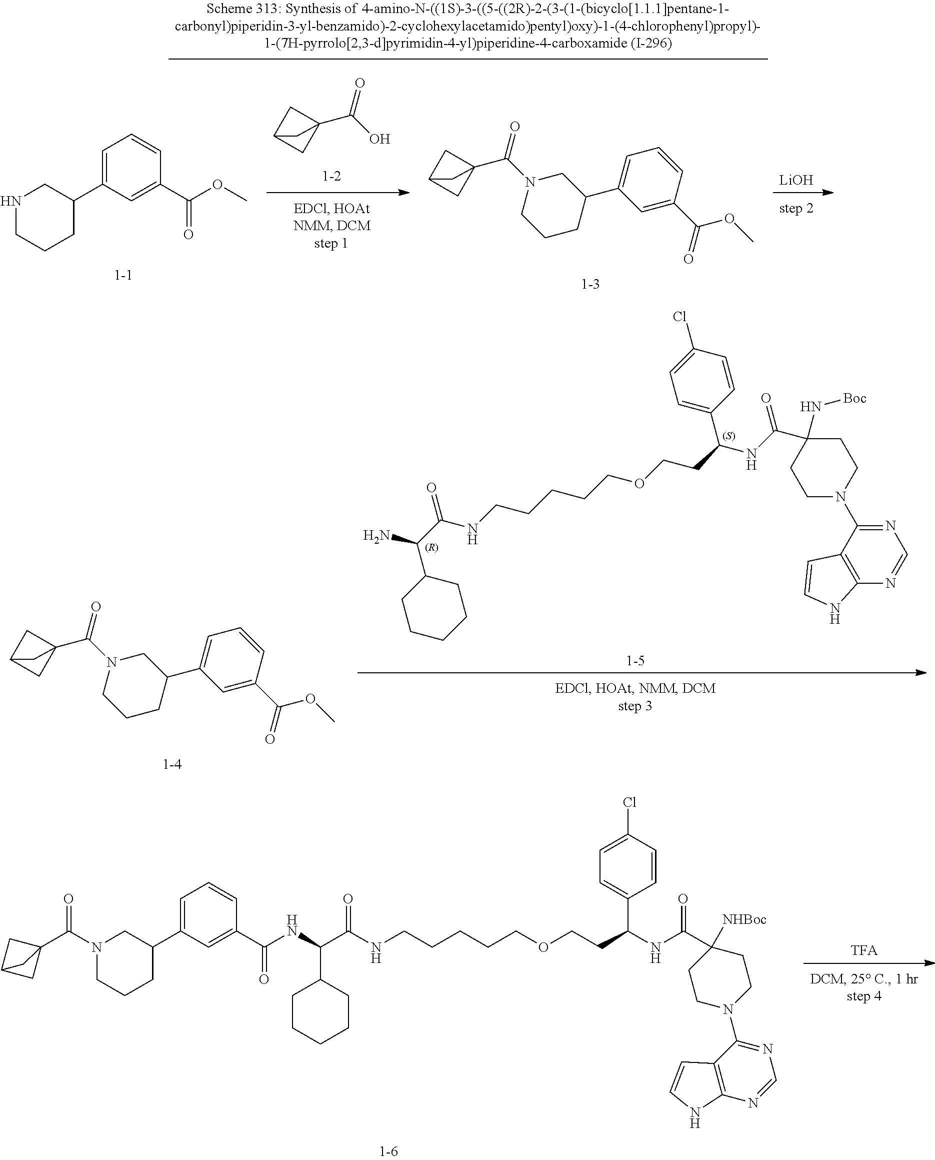

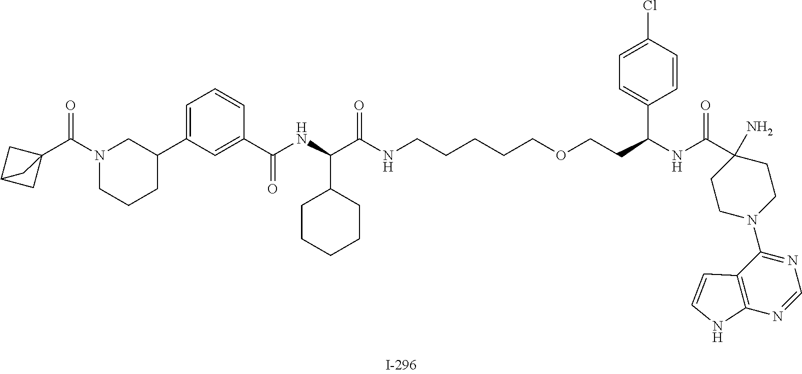 [SMILES: NC1(C(=O)N[C@@H](CCOCCCCCNC(=O)[C@H](NC(=O)c2cccc(C3CCCN(C(=O)C45CC(C4)C5)C3)c2)C2CCCCC2)c2ccc(Cl)cc2)CCN(c2ncnc3[nH]ccc23)CC1]

To a solution of 1-2 (241.14 mg, 2.15 mmol, 1.1 eq), EDCI (749.59 mg, 3.91 mmol, 2 eq), HOAt (133.06 mg, 977.56 μmol, 136.75 μL, 0.5 eq), NMM (988.80 mg, 9.78 mmol, 1.07 mL, 5 eq) in DCM (5 mL) was added 1-1 (500 mg, 1.96 mmol, 1 eq, HCl salt). Then the mixture was stirred at 25° C. for 1 hr. The reaction mixture was poured into water (20 mL) and extracted with DCM (20 mL*2), the organic layer was washed with brine (20 mL) and dried over Na₂SO₄. Then concentrated to get the crude product. The crude product was purified by flash silica gel column chromatography (SiO₂, PE:EA=10:1 to 3:1) to get 1-3 (600 mg, 1.91 mmol, 97.63% yield, 99.7% purity) as colorless oil. LCMS: R^{t}=0.526 min, [M+H]⁺=350.2.

### Step 2: Synthesis of 1-4

To a solution of 1-3 (580 mg, 1.85 mmol, 1 eq) in MeOH (3 mL) and H₂O (3 mL) was added LiOH·H₂O (388.28 mg, 9.25 mmol, 5 eq). Then the mixture was stirred at 25° C. for 1 hr. The reaction mixture was poured into water (20 mL) and adjust to pH=2 with 1N aq. HCl, and extracted with EA (20 mL*2). The organic layer was washed with brine (20 mL) and dried over Na₂SO₄, concentrated to get 1-4 ((500 mg, 1.63 mmol, 87.90% yield, 97.4% purity) as off-white solid. LCMS: R^{t}=0.472 min, [M+H]⁺=300.2.

### Step 3: Synthesis of 1-6

To a solution of 1-4 (59.60 mg, 199.11 μmol, 1.5 eq), EDCI (50.89 mg, 265.47 μmol, 2 eq), HOAt (9.03 mg, 66.37 μmol, 9.28 μL, 0.5 eq), NMM (67.13 mg, 663.68 μmol, 72.97 μL, 5 eq) in DCM (1 mL) was added 1-5 (100 mg, 132.74 μmol, 1 eq). Then the mixture was stirred at 25° C. for 1 hr. The reaction mixture was poured into water (20 mL) and extracted with DCM (20 mL*2), the organic layer was washed with brine (20 mL) and dried over Na₂SO₄, concentrated to get the crude product. The crude product was purified by flash silica gel column chromatography (SiO₂, PE:EA=10:1 to 3:1) to get 1-6 (40 mg, 34.71 μmol, 26.15% yield, 89.8% purity) as white solid. LCMS: R^{t}=0.580 min, [M+H]⁺=1034.6.

### Step 4: Synthesis of I-296

To a solution of 1-6 (40 mg, 38.66 μmol, 1 eq) in DCM (0.5 mL) was added TFA (0.1 mL). Then the mixture was stirred at 25° C. for 1 hr. The reaction mixture was concentrated to get the crude product. The crude product was purified by prep-HPLC (column: Phenomenex luna C18 150*25 mm*10 um; mobile phase: [water (TFA)-ACN]; gradient: 18%-48% B over 10 min), the eluent was concentrated and lyophilized to get I-296 (12.46 mg, 11.88 μmol, 30.74% yield, 100% purity, TFA salt) as a white solid by lyophilization. LCMS: R^{t}=0.511 min, [M+H]⁺=934.6. SFC: R^{t}=4.026 min, 5.066 min. ¹H NMR (400 MHZ, DMSO-d6) δ=12.06 (br s, 1H), 8.79 (br d, J=7.6 Hz, 1H), 8.48 (br s, 3H), 8.36-8.17 (m, 2H), 8.04 (br d, J=5.6 Hz, 1H), 7.87-7.70 (m, 2H), 7.50-7.36 (m, 4H), 7.34-7.27 (m, 3H), 6.79 (br d, J=1.6 Hz, 1H), 4.93 (br d, J=7.2 Hz, 1H), 4.71-4.57 (m, 2H), 4.43-4.10 (m, 4H), 3.56-3.51 (m, 2H), 3.33-3.21 (m, 4H), 3.18-3.01 (m, 3H), 2.70-2.59 (m, 2H), 2.40-2.32 (m, 2H), 2.18-2.05 (m, 5H), 2.04-1.88 (m, 5H), 1.84-1.67 (m, 7H), 1.58 (br d, J=8.8 Hz, 2H), 1.50-1.37 (m, 5H), 1.31-1.24 (m, 2H), 1.20-0.96 (m, 5H).

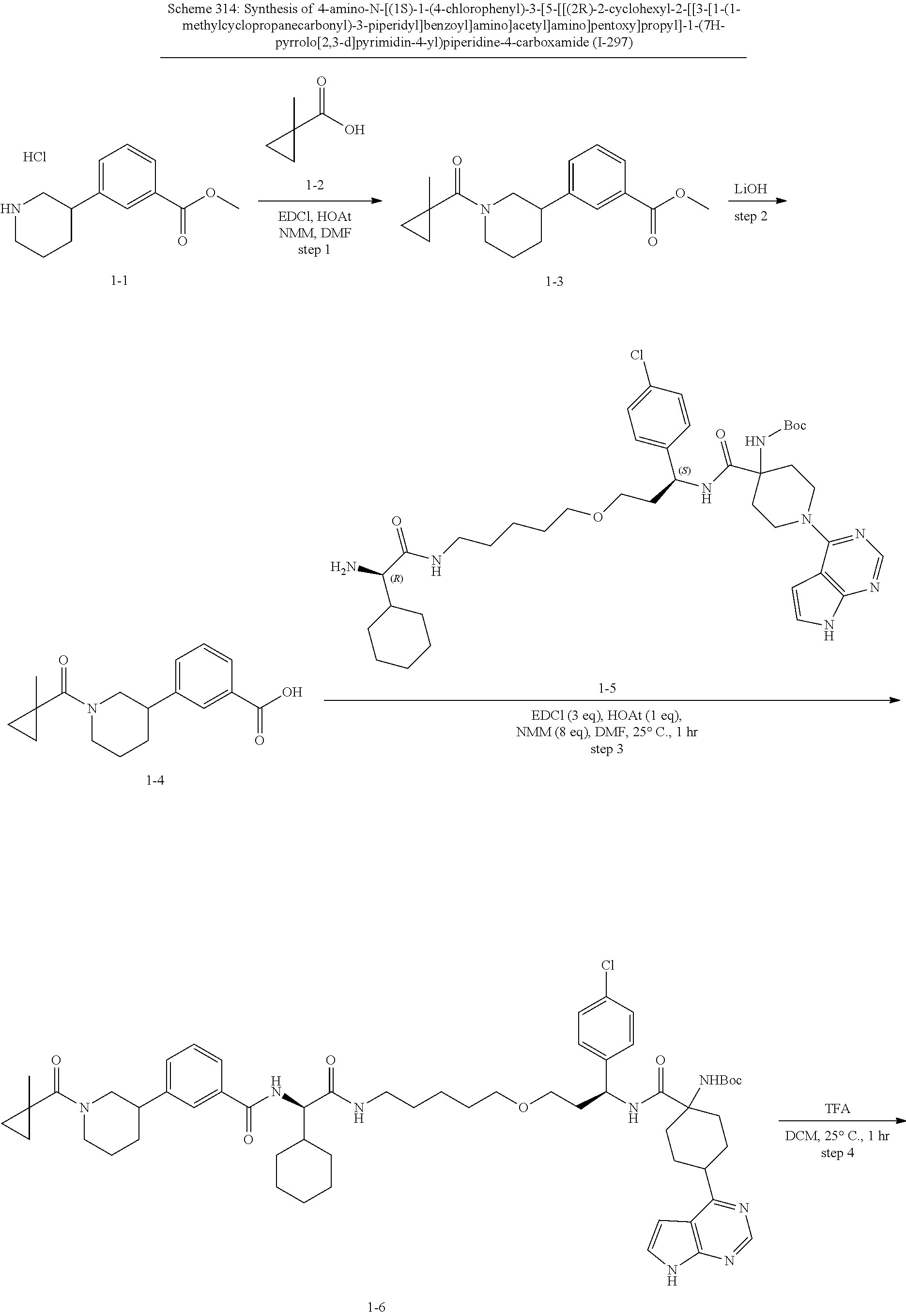

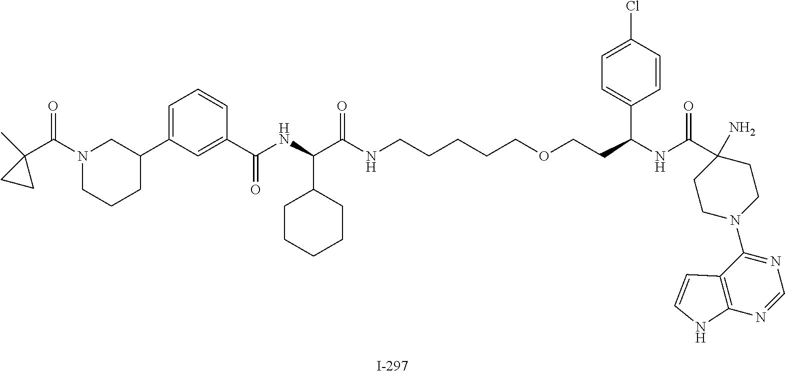 [SMILES: CC1(C(=O)N2CCCC(c3cccc(C(=O)N[C@@H](C(=O)NCCCCCOCC[C@H](NC(=O)C4(N)CCN(c5ncnc6[nH]ccc56)CC4)c4ccc(Cl)cc4)C4CCCCC4)c3)C2)CC1]

To a solution of 1-1 (600 mg, 2.35 mmol, 1 eq, HCl salt) and 1-2 (234.88 mg, 2.35 mmol, 1 eq) in DMF (6 mL) was added EDCI (899.51 mg, 4.69 mmol, 2 eq), NMM (1.19 g, 11.73 mmol, 1.29 mL, 5 eq) and HOAt (319.33 mg, 2.35 mmol, 328.20 μL, 1 eq). The mixture was stirred at 25° C. for 1 hr. The reaction mixture was extracted with EtOAc (3 mL*3). The combined organic layers were washed with brine (5 mL*2), dried over [Na₂SO₄], filtered and concentrated under reduced pressure to give 1-3 (700 mg, crude) as a white solid. LCMS: R^{t}=0.512 min, [M+H]⁺=302.1.

### Step 2: Synthesis of 1-4

To a solution of 1-3 (700 mg, 2.32 mmol, 1 eq) in THF (2 mL) was added LiOH·H₂O (292.40 mg, 6.97 mmol, 3 eq) in H₂O (2 mL) and MeOH (2 mL). The mixture was stirred at 25° C. for 1 hr. The reaction was acidified with 0.5M aq. HCl to pH<7, and extracted with EtOAc (5 mL*3). The combined organic layers were dried over [Na₂SO₄], filtered and concentrated under reduced pressure to give 1-4 (660 mg, crude) as a white solid. LCMS: R^{t}=0.448 min, [M+H]⁺=288.2. ¹H NMR (400 MHZ, DMSO-d₆) δ=13.43-12.21 (m, 1H), 7.88-7.78 (m, 2H), 7.55 (d, J=7.6 Hz, 1H), 7.50-7.41 (m, 1H), 4.40-4.18 (m, 2H), 2.99-2.87 (m, 1H), 2.85-2.62 (m, 2H), 1.97-1.89 (m, 1H), 1.83-1.69 (m, 2H), 1.57-1.39 (m, 1H), 1.23 (s, 3H), 0.89-0.75 (m, 2H), 0.60-0.44 (m, 2H).

### Step 3: Synthesis of 1-6

To a solution of 1-5 (100 mg, 132.74 μmol, 1 eq) and 1-4 (57.21 mg, 199.11 μmol, 1.5 eq) in DMF (1 mL) was added EDCI (76.34 mg, 398.21 μmol, 3 eq), NMM (107.41 mg, 1.06 mmol, 116.75 μL, 8 eq) and HOAt (18.07 mg, 132.74 μmol, 18.57 μL, 1 eq). The mixture was stirred at 25° C. for 1 hr. The reaction mixture was extracted with EtOAc (3 mL*3). The combined organic layers were washed with brine (5 ml*2), dried over [Na₂SO₄], filtered and concentrated under reduced pressure to give a residue. The residue was purified by flash silica gel chromatography (ISCO®; 12 g SepaFlash® Silica Flash Column, Eluent of 0˜50% Ethyl acetate/Petroleum ether gradient @ 20 mL/min). The eluent was concentrated to get 1-6 (80 mg, 78.22 μmol, 58.93% yield) as a white solid. LCMS: R^{t}=0.567 min, [M+H]⁺=1022.9.

### Step 4: Synthesis of I-297

To a solution of 1-6 (80 mg, 78.22 μmol, 1 eq) in DCM (1.5 mL) was added TFA (767.50 mg, 6.73 mmol, 0.5 mL, 86.05 eq). The mixture was stirred at 25° C. for 1 hr. The reaction mixture was concentrated under the pressure to give residue. The residue was purified by prep-HPLC (column: Phenomenex luna C18 150*25 mm*10 um; mobile phase: [water (TFA)-ACN]; gradient: 18%-48% B over 10 min). The eluent was concentrated and lyophilized to get I-297 (19 mg, 18.33 μmol, 23.43% yield, 100% purity; TFA salt) as a white solid. LCMS: R^{t}=0.501 min, [M+H]⁺=922.6. SFC: R^{t}=1.084, 1.310 min. ¹H NMR (400 MHz, DMSO-d₆) δ=12.33-11.83 (m, 1H), 8.87-8.76 (m, 1H), 8.50 (s, 2H), 8.28-8.20 (m, 2H), 8.06-8.03 (m, 1H), 7.81-7.70 (m, 2H), 7.46-7.36 (m, 4H), 7.32-7.27 (m, 3H), 6.78 (s, 1H), 4.98-4.88 (m, 1H), 4.68-4.51 (m, 2H), 4.35-4.24 (m, 3H), 3.34-3.19 (m, 5H), 3.12-3.07 (m, 1H), 3.04-2.99 (m, 1H), 2.91-2.81 (m, 1H), 2.67 (d, J=1.6 Hz, 1H), 2.39-2.32 (m, 2H), 1.98-1.84 (m, 4H), 1.82-1.73 (m, 5H), 1.70-1.64 (m, 2H), 1.62-1.55 (m, 2H), 1.50-1.24 (m, 8H), 1.23 (s, 3H), 1.19-0.89 (m, 6H), 0.87-0.75 (m, 3H), 0.53 (br s, 2H).

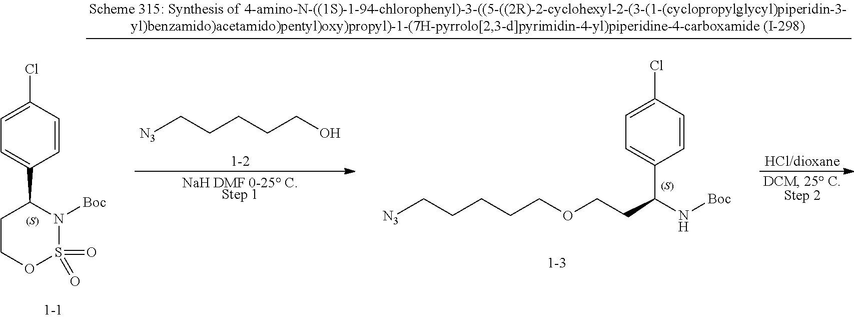

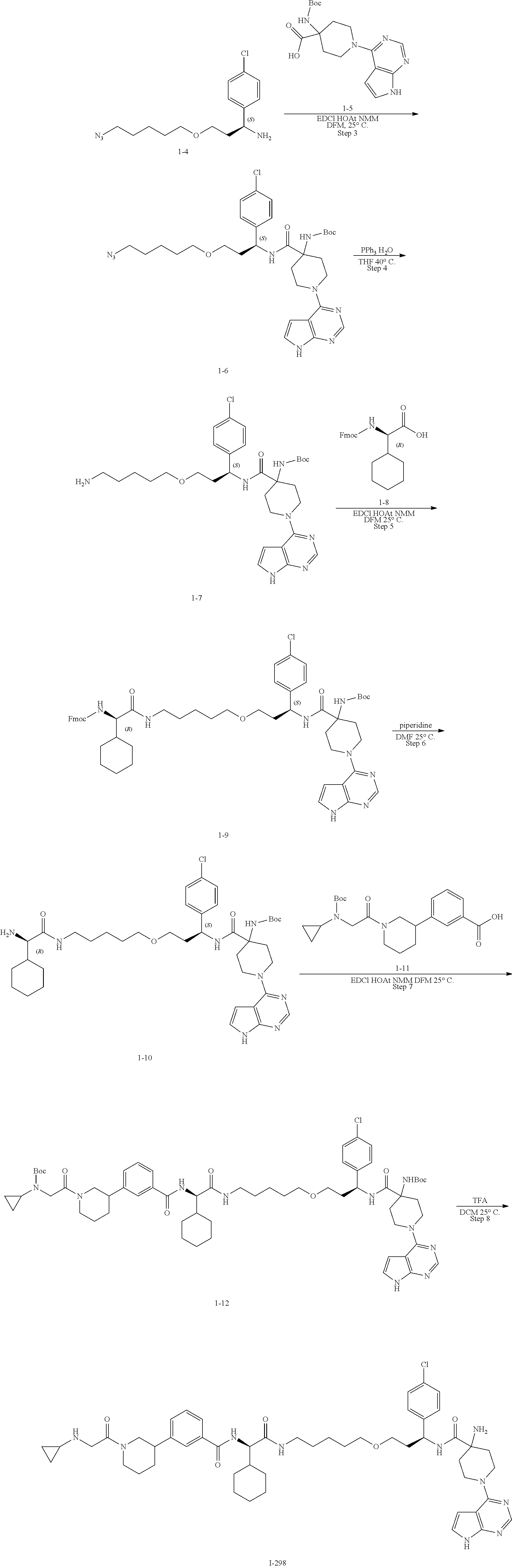

A solution of 1-2 (891.24 mg, 6.90 mmol, 1.2 eq) in DMF (20 mL) was degassed and purged with N₂ for 3 times. And then NaH (689.96 mg, 17.25 mmol, 60% purity, 3 eq) was added dropwise at 0° C., and purged with N₂ for 3 times. The mixture was stirred at 25° C. for 0.2 hr, then 1-1 (2 g, 5.75 mmol, 1 eq) was added dropwise at 0° C., and purged with N₂ for 3 times. The resulting mixture was stirred at 25° C. for 1.8 hr. The reaction was diluted with aq. sat. NH₄Cl (10 mL), extracted with ethyl acetate (10 mL*3), the organic phase was washed with saturated aqueous NaCl (10 mL). Then dried over [Na₂SO₄], filtered and concentrated under reduced pressure to give a residue. The residue was purified by reversed phase (26.8*125 mm, 120 g of XB—C18, 20-40 μm, 120 Å) Mobile phase: A for H₂O (0.1% NH₃·H₂O v/v) and B for acetonitrile; Gradient: B 30%-50% in 30 min: Flow rate: 60 ml/min; Column temperature: R.T. Wavelength: 220 nm/254 nm), after purification, then the eluent was concentrated to remove organic solvents and lyophilized to get 1-3 (520 mg, 1.19 mmol, 20.68% yield, 90.76% purity) as a white solid. LCMS: R^{t}=0.626 min [M+Na]⁺=419.1. SFC: R^{t}=0.985 min, ee %>99%.

### Step 2: Synthesis of 1-4

To a solution of 1-3 (500 mg, 1.26 mmol, 1 eq) in DCM (5 mL) was added HCl/dioxane (2 M, 5 mL, 7.94 eq). The mixture was stirred at 25° C. for 1 hr. The reaction mixture was concentrated under reduced pressure to give 1-4 (430 mg, crude, HCl salt) as white solid. LCMS: R^{t}=0.457 min, [M+H]⁺=297.1.

### Step 3: Synthesis of 1-6

To a solution of 1-4 (430 mg, 1.29 mmol, 1 eq, HCl salt), 1-5 (466.31 mg, 1.29 mmol, 1 eq) in DMF (5 mL) was added EDCI (742.06 mg, 3.87 mmol, 3 eq) and HOAt (175.62 mg, 1.29 mmol, 180.50 μL, 1 eq), NMM (1.04 g, 10.32 mmol, 1.13 mL, 8 eq). The mixture was stirred at 25° C. for 1 hr. The reaction mixture was quenched by addition water (5 mL) at 25° C., and then extracted with EA (5 mL*3). The combined organic layers were dried over Na₂SO₄, filtered and concentrated under reduced pressure to give a residue. The residue was purified by flash silica gel chromatography (ISCO®; 40 g SepaFlash R) Silica Flash Column, Eluent of 0˜100% Ethyl acetate/Petroleum ether gradient @ 60 mL/min). The eluent was concentrated to get 1-6 (650 mg, 941.53 μmol, 72.97% yield, 92.73% purity) as white solid. LCMS: R^{t}=0.573 min, [M+H]⁺=640.3. SFC: R^{t}=1.503 min, ee %>99%.

### Step 4: Synthesis of 1-7

To a solution of PPh₃ (270.41 mg, 1.03 mmol, 1.1 eq) in THF (6 mL) and H₂O (50.65 mg, 2.81 mmol, 50.65 μL, 3 eq) was added 1-6 (600 mg, 937.24 μmol, 1 eq) in THF (6 mL). The mixture was stirred at 40° C. for 1 hr. The reaction mixture was concentrated under reduced pressure to give a residue. The residue was purified by reversed phase (26.8*125 mm, 80 g of XB—C18, 20-40 μm, 120 Å) Mobile phase: A for H₂O (0.1% NH₃·H₂O v/v) and B for acetonitrile; Gradient: B 30%-50% in 30 min: Flow rate: 60 ml/min; Column temperature: R.T. Wavelength: 220 nm/254 nm), The eluent was concentrated and lyophilized to afford 1-7 (400 mg, 644.77 μmol, 68.79% yield, 99% purity) as white solid. LCMS: R^{t}=0.432 min, [M+H]⁺=614.3. SFC: R^{t}=1.875 min, ee %>99%.

### Step 5: Synthesis of 1-9

To a solution of 1-7 (400 mg, 651.28 μmol, 1 eq), 1-8 (247.13 mg, 651.28 μmol, 247.13 μL, 1 eq) in DMF (5 mL) was added EDCI (374.55 mg, 1.95 mmol, 3 eq) and HOAt (88.65 mg, 651.28 μmol, 91.11 μL, 1 eq), NMM (329.37 mg, 3.26 mmol, 358.02 μL, 5 eq). The mixture was stirred at 25° C. for 1 hr. The reaction mixture was quenched by addition water (5 mL) at 25° C., and then extracted with EA (5 mL*3). The combined organic layers were dried over Na₂SO₄, filtered and concentrated under reduced pressure to give 1-9 (500 mg, 491.33 μmol, 75.44% yield, 95.87% purity) as yellow solid. LCMS: R^{t}=0.618 min, [M+H]⁺=975.6.

### Step 6: Synthesis of 1-10

To a solution of 1-9 (500 mg, 512.50 μmol, 1 eq) in DCM (5 mL) was added PIPERIDINE (862.20 mg, 10.13 mmol, 1 mL, 19.76 eq). The mixture was stirred at 25° C. for 1 hr. The reaction mixture was quenched by addition water (5 mL) at 25° C., and then extracted with EA (5 mL*3). The combined organic layers were dried over Na₂SO₄, filtered and concentrated under reduced pressure to give a residue. The residue was purified by reversed phase (26.8*125 mm, 80 g of XB—C18, 20-40 μm, 120 Å) Mobile phase: A for H₂O (0.1% NH₃·H₂O v/v) and B for acetonitrile; Gradient: B 30%-50% in 30 min: Flow rate: 60 ml/min; Column temperature: R.T. Wavelength: 220 nm/254 nm), the eluent was concentrated and lyophilized to afford 1-10 (340 mg, 441.29 μmol, 86.10% yield, 97.78% purity) as white solid. LCMS: R^{t}=0.459 min, [M+H]⁺=753.6. SFC: R^{t}=1.436 min, ee %>99%.

### Step 7: Synthesis of 1-12

To a solution of 1-10 (60 mg, 79.64 μmol, 1 eq) and 1-11 (48.08 mg, 119.46 μmol, 1.5 eq) in DMF (0.5 mL) was added HOAt (10.84 mg, 79.64 μmol, 11.14 μL, 1 eq) and EDCI (45.80 mg, 238.93 μmol, 3 eq), NMM (64.44 mg, 637.14 μmol, 70.05 μL, 8 eq). The mixture was stirred at 25° C. for 1 hr. The reaction mixture was quenched by addition water (5 ml) at 25° C., and then extracted with EA (5 mL*3). The combined organic layers were dried over Na₂SO₄, filtered and concentrated under reduced pressure to give a residue. The residue was purified by flash silica gel chromatography (ISCO®; 20 g SepaFlash® Silica Flash Column, Eluent of 0˜100% Ethyl acetate/Petroleum ether gradient @ 40 mL/min). The eluent was concentrated to get 1-12 (70 mg, 55.28 μmol, 69.41% yield, 89.85% purity) as yellow solid. LCMS: R^{t}=0.611 min, [M+H]⁺=1137.7. SFC: R^{t}=2.009 min.

### Step 8: Synthesis of I-298

To a solution of 1-12 (60 mg, 52.73 μmol, 1 eq) in DCM (0.6 mL) was added TFA (2 M, 200.00 μL, 7.59 eq). The mixture was stirred at 25° C. for 1 hr. The reaction mixture was concentrated under reduced pressure to give a residue The combined crude product was purified by prep-HPLC (column: Phenomenex luna C18 150*25 mm*10 um; mobile phase: [water (TFA)-ACN]; gradient: 8%-38% B over 10 min), the eluent was concentrated and lyophilized to afford I-298 (65 mg, TFA salt) as white solid. LCMS: R^{t}=0.448 min, [M+H]⁺=937.6. SFC: R^{t}=3.955 min. ¹H NMR (400 MHZ, METHANOL-d4) δ=8.38 (s, 1H), 7.84-7.67 (m, 2H), 7.54-7.39 (m, 2H), 7.37 (d, J=3.6 Hz, 1H), 7.30 (s, 4H), 6.96-6.85 (m, 1H), 5.09 (m, 1H), 4.68-4.42 (m, 3H), 4.37-4.11 (m, 3H), 3.93-3.69 (m, 3H), 3.44-3.34 (m, 3H), 3.29-3.18 (m, 3H), 3.06-2.70 (m, 3H), 2.65-2.45 (m, 2H), 2.17 (d, J=15.2 Hz, 1H), 2.11-1.95 (m, 4H), 1.94-1.62 (m, 9H), 1.54 (m, 4H), 1.45-1.35 (m, 2H), 1.35-1.23 (m, 3H), 1.22-1.02 (m, 3H), 1.00-0.85 (m, 4H).

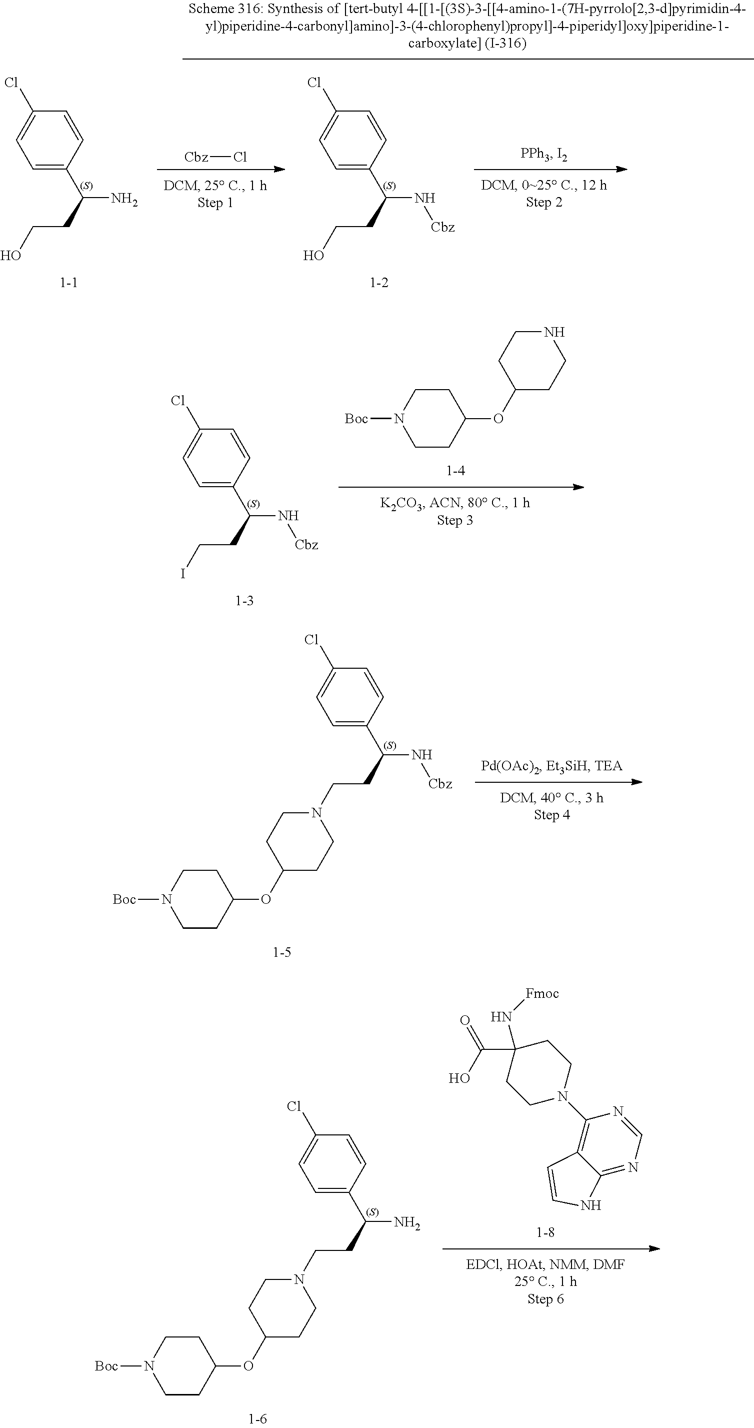

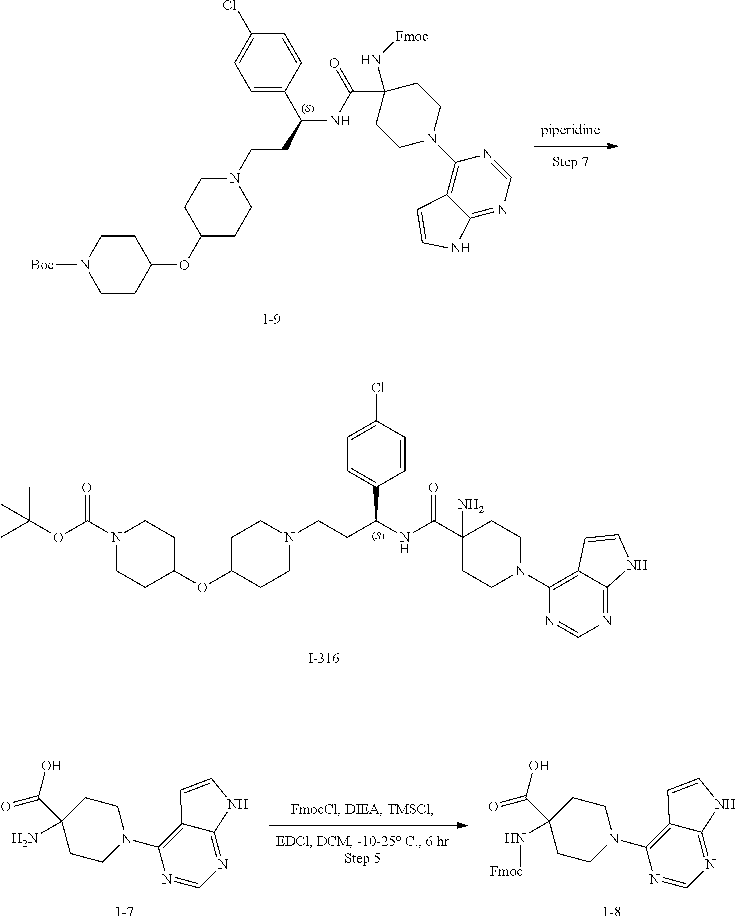

### Step 1: Synthesis of 1-2.

To a mixture of 1-1 (1 g, 5.39 mmol, 1 eq) in DCM (10 mL) was added CbzCl (1.10 g, 6.46 mmol, 922.74 μL, 1.2 eq) and TEA (817.58 mg, 8.08 mmol, 1.12 mL, 1.5 eq) at 0° C., the mixture was stirred at 25° C. for 1 h. The reaction mixture was diluted with H₂O (10 mL) and extracted with DCM (10 mL×3), the combined organic layers were dried over Na₂SO₄, filtered, and concentrated to give a residue. The residue was purified by column chromatography (SiO₂, PE/EA=1/0 to 1/1) and concentrated to give the product. 1-2 (370 mg, 1.16 mmol, 21.48% yield, N/A purity) was obtained as a white solid. LCMS (Method D): R^{t}=0.460 min, [M+H]⁺=320.0.

### Step 2: Synthesis of 1-3.

A solution of PPh₃ (524.93 mg, 2.00 mmol, 2 eq), I₂ (507.96 mg, 2.00 mmol, 403.14 μL, 2 eq) and imidazole (272.49 mg, 4.00 mmol, 4 eq) in DCM (3 mL) was stirred at 0° C. for 0.5 h, then the solution of 1-2 (320 mg, 1.00 mmol, 1 eq) in DCM (1 mL) was added and stirred at 25° C. for 11.5 hr. The reaction mixture was diluted with H₂O (5 mL) and extracted with DCM (5 mL×3), the combined organic layers were dried over Na₂SO₄, filtered, and concentrated to give a residue. The residue was purified by column chromatography (SiO₂, PE/EA=1/0 to 1/1) and concentrated to give the product. 1-3 (355 mg, 826.20 μmol, 82.56% yield, N/A purity) was obtained as a white solid. LCMS (Method D): R^{t}=0.447 min, [M+H]⁺=429.9.

### Step 3: Synthesis of 1-5.

To a mixture of 1-3 (300 mg, 698.20 μmol, 1 eq) in ACN (3 mL) was added 1-4 (218.42 mg, 768.02 μmol, 1.1 eq) and K₂CO₃ (289.48 mg, 2.09 mmol, 3 eq) at 25° C., the mixture was stirred at 80° C. for 1 h. The reaction mixture was diluted with H₂O (5 mL) and extracted with DCM (5 mL×3), the combined organic layers were dried over Na₂SO₄, filtered, and concentrated to give a residue. The residue was purified by column chromatography (SiO₂, EA/MeOH=1/0 to 1/1) and concentrated to give the product. 1-5 (450 mg, crude) was obtained as a white solid. LCMS (Method D): R^{t}=0.388 min, [M+H]⁺=586.3.

### Step 4: Synthesis of 1-6.

To a mixture of 1-5 (400 mg, 682.41 μmol, 1 eq) in DCM (4 mL) was added Pd(OAc)₂ (76.60 mg, 341.20 μmol, 0.5 eq), TEA (207.16 mg, 2.05 mmol, 284.95 μL, 3 eq) and Et₃SiH (952.18 mg, 8.19 mmol, 1.31 mL, 12 eq) at 25° C., the mixture was degassed and purged with N₂ for 3 times and stirred at 40° C. for 3 h. The reaction mixture was filtered, and the filtrate was diluted with H₂O (5 mL), extracted with EtOAc (5 mL×3), the combined organic layers were dried over Na₂SO₄, filtered, and concentrated to give a residue. The residue was purified by reverse-phase HPLC (0.1% FA condition) and lyophilized to give the product. 1-6 (185 mg, 409.27 μmol, 59.97% yield, N/A purity) was obtained as a white solid. LCMS (Method D): R^{t}=0.261 min, [M+H]⁺=452.0.

### Step 5: Synthesis of 1-8.

To a mixture of 1-7 (1 g, 3.36 mmol, 1 eq, HCl) in DCM (10 mL) was added DIEA (1.30 g, 10.08 mmol, 1.75 mL, 3 eq) at 25° C. for 0.5 h, then TMSCl (729.76 mg, 6.72 mmol, 852.53 μL, 2 eq) was added and stirred at 40° C. for 3 h, during which N₂ was bubbled for 30 seconds every 30 minutes to remove the hydrogen chloride gas generated by the reaction. The reaction mixture was cooled to −10° C., Fmoc-Cl (868.88 mg, 3.36 mmol, 1 eq) and EDCI (64.39 mg, 335.86 μmol, 0.1 eq) were added, and stirred at 25° C. for 3 h. During this period, N₂ was bubbled every 30 minutes for 30 seconds to remove the hydrogen chloride gas generated by the reaction. The reaction mixture was quenched by H₂O (10 mL) and filtered: the filtrate was concentrated to give a residue. The residue was purified by reversed phase column (0.1% FA condition) and lyophilized to give the product. 1-8 (75 mg, 155.11 μmol, 4.62% yield, N/A purity) was obtained as a white solid. LCMS (Method D): R^{t}=0.330 min, [M+H]′=484.2. ¹H NMR (400 MHZ, METHANOL-d4) δ=8.15 (s, 1H), 7.80 (d, J=7.6 Hz, 2H), 7.70 (d, J=7.2 Hz, 2H), 7.42-7.36 (m, 2H), 7.34-7.28 (m, 2H), 7.16 (d, J=3.6 Hz, 1H), 6.65 (d, J=3.6 Hz, 1H), 4.50-4.36 (m, 4H), 4.25 (t, J=6.8 Hz, 1H), 3.59-3.46 (m, 2H), 2.26-2.08 (m, 4H).

### Step 6: Synthesis of 1-9.

To a mixture of 1-6 (70 mg, 154.86 μmol, 1 eq) in DMF (1 mL) was added 1-8 (74.88 mg, 154.86 μmol, 1 eq), EDCI (89.06 mg, 464.57 μmol, 3 eq), HOAt (21.08 mg, 154.86 μmol, 21.66 μL, 1 eq) and NMM (78.32 mg, 774.29 μmol, 85.13 μL, 5 eq) at 25° C., the mixture was stirred at 25° C. for 1 h. The reaction mixture was diluted with H₂O (2 mL) and extracted with DCM (1 mL×3), the combined organic layers were dried over Na₂SO₄, filtered, and concentrated to give a residue. The residue was purified by column chromatography (SiO₂, EA/MeOH=1/0 to 6/4) and concentrated to give the product. 1-9 (80 mg, 87.19 μmol, 56.30% yield) was obtained as a yellow solid. LCMS: R^{t}=0.389 min, [M+H]⁺=917.6.

### Step 7: Synthesis of I-316.

To a mixture of 1-9 (70 mg, 76.29 μmol, 1 eq) in DMF (0.7 mL) was added piperidine (12.99 mg, 152.58 μmol, 15.07 μL, 2 eq) at 25° C., the mixture was stirred at 25° C. for 0.5 h. The reaction mixture was diluted with H₂O (1 mL) and extracted with DCM (1 mL×3), the combined organic layers were dried over Na₂SO₄, filtered, and concentrated to give a residue. The residue was purified by prep-HPLC (column: CD07-Daisogel SP-100-8-ODS-PK 150*25*10 um; mobile phase: [water (NH₄HCO₃)-ACN]; gradient: 36%-66% B over 11 min) and lyophilized to give the product. I-316 (16.25 mg, 22.66 μmol, 29.71% yield, 96.974% purity) was obtained as a white solid. LCMS: R^{t}=0.289 min, [M+H]′=695.5. SFC: R^{t}=1.261 min. ¹H NMR (400 MHZ, METHANOL-d4) δ=8.12 (s, 1H), 7.32 (d, J=1.2 Hz, 3H), 7.12 (d, J=3.6 Hz, 1H), 6.62 (d, J=3.6 Hz, 1H), 4.92-4.89 (m, 1H), 4.54-4.41 (m, 2H), 3.77-3.69 (m, 2H), 3.69-3.58 (m, 3H), 3.53-3.45 (m, 1H), 3.15-3.05 (m, 2H), 2.83-2.66 (m, 2H), 2.40-2.29 (m, 2H), 2.28-2.16 (m, 3H), 2.15-2.08 (m, 1H), 2.08-1.93 (m, 2H), 1.90-1.82 (m, 2H), 1.82-1.75 (m, 2H), 1.67-1.52 (m, 4H), 1.45 (s, 9H), 1.44-1.37 (m, 2H).

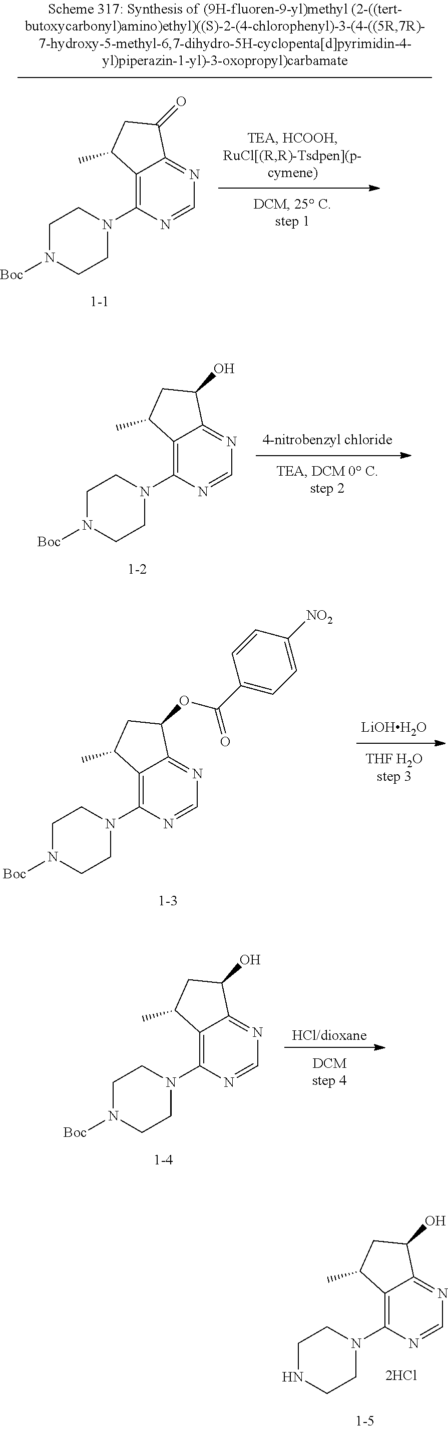

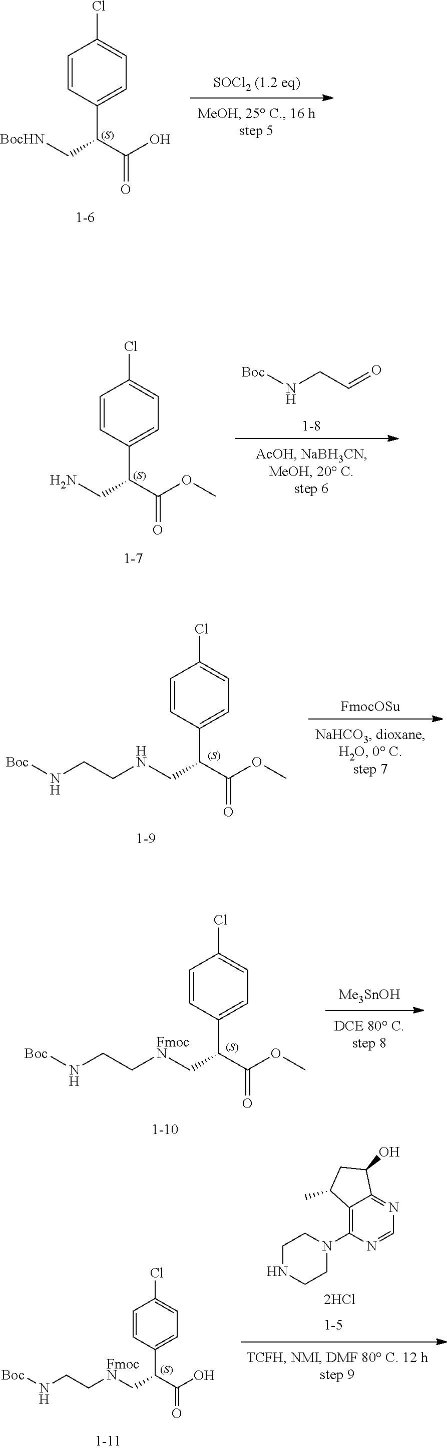

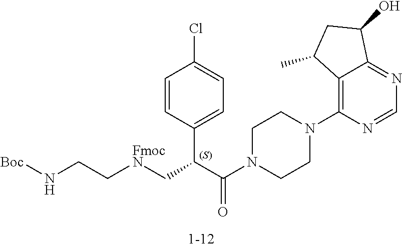

### Step 1: Synthesis of 1-2

A mixture of 1-1 (18 g, 54.15 mmol, 1 eq), TEA (5.81 g, 57.40 mmol, 7.99 mL, 1.06 eq), HCOOH (3.09 g, 64.33 mmol, 2.53 mL, 1.19 eq) in DCM (400 mL) was degassed and purged with N₂ for 3 times, and then the mixture was stirred at 25° C. for 15 min under N₂ atmosphere. Then chloro-[p-tolylsulfonyl-[rac-(1R,2R)-2-amino-1,2-diphenyl-ethyl]amino]ruthenium; 1-isopropyl-4-methyl-benzene (1.72 g, 2.71 mmol, 0.05 eq) was added in, the mixture was stirred at 25° C. for 75 min under N₂ atmosphere. The reaction was diluted with H₂O (200 mL), extracted with ethyl acetate 600 mL (300 mL*3). The organic phase was washed with saturated aqueous NaCl (100 mL), dried over [Na₂SO₄], filtered and concentrated under reduced pressure to give a residue. The crude product was no further purification and using for next step directly. 1-2 (18 g, crude) was obtained as a brown solid. LCMS: Rt=0.417 min, [M+H]⁺=335.2

### Step 2: Synthesis of 1-3

To a solution of 4-nitrobenzoyl chloride (29.96 g, 161.48 mmol, 3 eq) and 1-2 (18 g, 53.83 mmol, 1 eq) in DCM (200 mL) was added TEA (16.34 g, 161.48 mmol, 22.48 mL, 3 eq). The mixture was stirred at 0° C. for 1 hr. The reaction was diluted with H₂O (200 mL), extracted with ethyl acetate 600 mL (200 mL*3). The organic phase was washed with saturated aqueous NaCl (200 mL), dried over [Na₂SO₄], filtered and concentrated under reduced pressure to give a residue. The residue was purified by flash silica gel chromatography (ISCO®; 660 g SepaFlash® Silica Flash Column, Eluent of 0˜80% Ethyl acetate/Petroleum ether gradient (@; 40 mL/min). The eluent was concentrated to afford product. 1-3 (28 g, crude) was obtained as a brown oil. LCMS: Rt=0.515 min, [M+H]⁺=484.2. ¹H NMR (400 MHZ, CHLOROFORM-d) δ=8.64 (s, 1H), 8.23 (s, 4H), 6.39 (t, J=7.2 Hz, 1H), 3.92-3.77 (m, 2H), 3.75-3.58 (m, 5H), 3.56-3.45 (m, 2H), 2.48-2.26 (m, 2H), 1.50 (s, 9H), 1.29 (d, J=6.8 Hz, 3H)

### Step 3: Synthesis of 1-4

To a solution of 1-3 (23 g, 47.57 mmol, 1 eq) in THF (130 mL) and H₂O (100 mL) was added LiOH·H₂O (5.99 g, 142.70 mmol, 3 eq). The mixture was stirred at 25° C. for 1 hr. The reaction was concentrated under reduced pressure to give a residue. Then water phase was washed with saturated aqueous NaHCO₃ (20 mL), extracted with EA 60 mL (20 mL*3). The organic layers were dried over [Na₂SO₄], filtered and concentrated under reduced pressure to give a residue, which was the crude product. The crude product was no further purification and using for next step directly. 1-4 (18 g, crude) was obtained as brown oil. LCMS: Rt=0.412 min, [M+H]⁺=335.2. ¹H NMR (400 MHz, CHLOROFORM-d) δ=8.53 (s, 1H), 5.24-5.00 (m, 1H), 3.87-3.72 (m, 2H), 3.71-3.61 (m, 2H), 3.60-3.40 (m, 5H), 2.31-2.10 (m, 2H), 1.49 (s, 9H), 1.20 (br d, J=6.2 Hz, 3H)

### Step 4: Synthesis of 1-5

The mixture of 1-4 (18 g, 53.83 mmol, 1 eq) in 2M HCl/dioxane (90 mL) and DCM (90 mL) was stirred at 25° C. for 4 hr. The reaction was filtered and concentrated under reduced pressure to give a residue. The crude product was purified by re-crystallization from DCM (500 mL) at 25° C. Then filtered and concentrated under reduced pressure to give a residue. 1-5 (10.8 g, 34.03 mmol, 63.22% yield, 96.794% purity, 2HCl) was obtained as a gray solid. LCMS: Rt=0.331 min, [M+H]⁺=235.2. SFC: Rt=2.453 min. ¹H NMR (400 MHZ, DMSO-d₆) δ=10.10 (br s, 2H), 8.83 (s, 1H), 5.32 (t, J=8.0 Hz, 1H), 4.42-4.21 (m, 2H), 4.12-4.07 (m, 10.8 Hz, 2H), 3.69 (br t, J=6.8 Hz, 1H), 3.40-3.17 (m, 4H), 2.22-1.99 (m, 2H), 1.13 (d, J=6.8 Hz, 3H)

### Step 5: Synthesis of 1-7

To a solution of 1-6 (30 g, 100.08 mmol, 1 eq) in MeOH (300 mL) was added SOCl₂ (14.29 g, 120.10 mmol, 8.72 mL, 1.2 eq). The mixture was stirred at 25° C. for 16 hr. The reaction was filtered and concentrated under reduced pressure to give a residue. The crude product was no further purification and using for next step directly. 1-7 (20 g. 88.12 mmol, 88.05% yield, 94.14% purity) was obtained as a white solid. LCMS: Rt=0.385 min, [M+H]⁺=214.2. ¹H NMR (400 MHZ, METHANOL-d4) δ=7.45-7.39 (m, 2H), 7.36-7.30 (m, 2H), 4.10-4.06 (m, 1H), 3.73 (s, 3H), 3.62-3.57 (m, 1H), 3.26-3.23 (m, 1H)

### Step 6: Synthesis of 1-9

To a solution of 1-7 (18.6 g, 87.05 mmol, 1 eq) and 1-8 (16.63 g, 104.46 mmol, 1.2 eq) in MeOH (200 mL) and AcOH (5.23 g, 87.05 mmol, 4.98 mL, 1 eq). The mixture was stirred at 20° C. for 0.5 hr. Then NaBH₃CN (10.94 g, 174.11 mmol, 2 eq) was added in the mixture. The mixture was stirred at 20° C. for 1.5 hr. The reaction was diluted with H₂O (50 mL), extracted with DCM 300 mL (100 mL*3), the organic phase was washed with saturated aqueous NaCl (50 mL), dried over [Na₂SO₄], filtered and concentrated under reduced pressure to give a residue. It was used into the next step without further purification. 1-9 (40 g, crude) was obtained as a yellow oil. LCMS: Rt=0.458 min, [M+H]⁺=357.1. ¹H NMR (400 MHZ, METHANOL-d4) δ=7.43-7.38 (m, 2H), 7.33-7.31 (m, 2H), 3.72 (s, 3H), 3.15-3.02 (m, 4H), 2.73-2.60 (m, 1H), 2.58-2.46 (m, 1H), 1.45 (br s, 10H)

### Step 7: Synthesis of 1-10

To a solution of 1-9 (30 g, 84.07 mmol, 1 eq) in dioxane (60 mL) and H₂O (180 mL) was added NaHCO₃ (17.66 g, 210.18 mmol, 8.18 mL, 2.5 eq) at 0° C. Then a solution of (2,5-dioxopyrrolidin-1-yl) 9H-fluoren-9-ylmethyl carbonate (34.03 g. 100.88 mmol, 1.2 eq) in dioxane (60 mL) was added in the mixture. The mixture was stirred at 0° C. for 1 hr. The reaction was diluted with H₂O (200 mL), extracted with ethyl acetate 600 mL (200 mL*3). The organic phase was washed with saturated aqueous NaCl (5 mL), dried over [Na₂SO₄], filtered and concentrated under reduced pressure to give a residue. The residue was purified by flash silica gel chromatography (ISCO®; 660 g SepaFlash® Silica Flash Column, Eluent of 0˜100% Ethyl acetate/Petroleum ether gradient (a, 200 mL/min). The eluent was concentrated to afford product. 1-10 (28 g, 48.35 mmol, 57.51% yield, 100% purity) was obtained as yellow oil. LCMS: Rt=0.649 min, [M+H-Boc]=479.2. SFC: Rt=1.237 min. ¹H NMR (400 MHZ, METHANOL-d4) δ=7.84-7.74 (m, 2H), 7.69-7.50 (m, 2H), 7.40 (d, J=7.2 Hz, 2H), 7.34-7.26 (m, 3H), 7.20 (d, J=8.0 Hz, 2H), 6.73 (d, J=7.6 Hz, 1H), 4.48 (br s, 1H), 4.26-4.21 (m, 1H), 3.93-3.75 (m, 1H), 3.73-3.67 (m, 2H), 3.66-3.58 (m, 3H), 3.57-3.35 (m, 1H), 3.12-3.04 (m, 2H), 2.67-2.62 (m, 1H), 2.55-2.47 (m, 1H), 1.45 (s, 9H)

### Step 8: Synthesis of 1-11

To a solution of 1-10 (10 g, 17.27 mmol, 1 eq) in DCE (100 mL) was added hydroxy (trimethyl) stannane (9.73 g, 53.81 mmol, 3.12 eq). The mixture was stirred at 80° C. for 12 hr. The reaction was cooled to 0° C., which was filtered and concentrated under reduced pressure to give a residue. And the filter cake which was quenched with Sat, aq. KF solution (200 mL). The residue was purified by flash silica gel chromatography (ISCO®; 220 g SepaFlash® Silica Flash Column, Eluent of 0˜100% Ethyl acetate/Petroleum ether gradient @ 100 mL/min). The eluent was concentrated to afford product. 1-11 (5.5 g, 9.52 mmol, 55.14% yield, 97.818% purity) was obtained as yellow oil. LCMS: Rt=0.632 min, [M+H-Boc]⁺=465.2. SFC: Rt=1.560 min

### Step 9: Synthesis of 1-12

To a solution of 1-11 (5.5 g, 9.73 mmol, 1 eq) and 1-5 (3.59 g, 11.68 mmol, 1.2 eq, 2HCl) in DMF (55 mL) was added TCFH (5.46 g, 19.47 mmol, 2 eq) and NMI (4.00 g, 48.67 mmol, 3.88 mL, 5 eq). The mixture was stirred at 80° C. for 12 hr. The reaction was diluted with H₂O (50 mL), extracted with ethyl acetate 300 mL (100 mL*3). The organic phase was washed with saturated aqueous NaCl (50 mL), dried over [Na₂SO₄], filtered and concentrated under reduced pressure to give a residue. The residue was purified by flash silica gel chromatography (ISCO®; 120 g SepaFlash® Silica Flash Column, Eluent of 0˜60% EA/MeOH gradient @ 100 mL/min). The eluent was concentrated to afford product. 1-12 (5.5 g, 6.68 mmol, 68.59% yield, 94.85% purity) was obtained as a white solid. LCMS: Rt=0.539 min, [M+H]⁺=781.5. SFC: Rt=1.881 min, ee %=92.798%. ¹H NMR (400 MHZ, METHANOL-d4) δ=8.43 (br d, J=12.8 Hz, 1H), 7.88-7.76 (m, 2H), 7.70-7.52 (m, 2H), 7.48-7.18 (m, 7H), 6.91-6.76 (m, 1H), 5.03-4.92 (m, 1H), 4.74 (br d, J=2.0 Hz, 1H), 4.56-4.35 (m, 1H), 4.29-4.21 (m, 1H), 3.94-3.60 (m, 4H), 3.59-3.33 (m, 6H), 3.29-2.99 (m, 5H), 2.82-2.66 (m, 1H), 2.25-2.03 (m, 2H), 1.39 (br s, 9H), 1.19-1.05 (m, 3H)

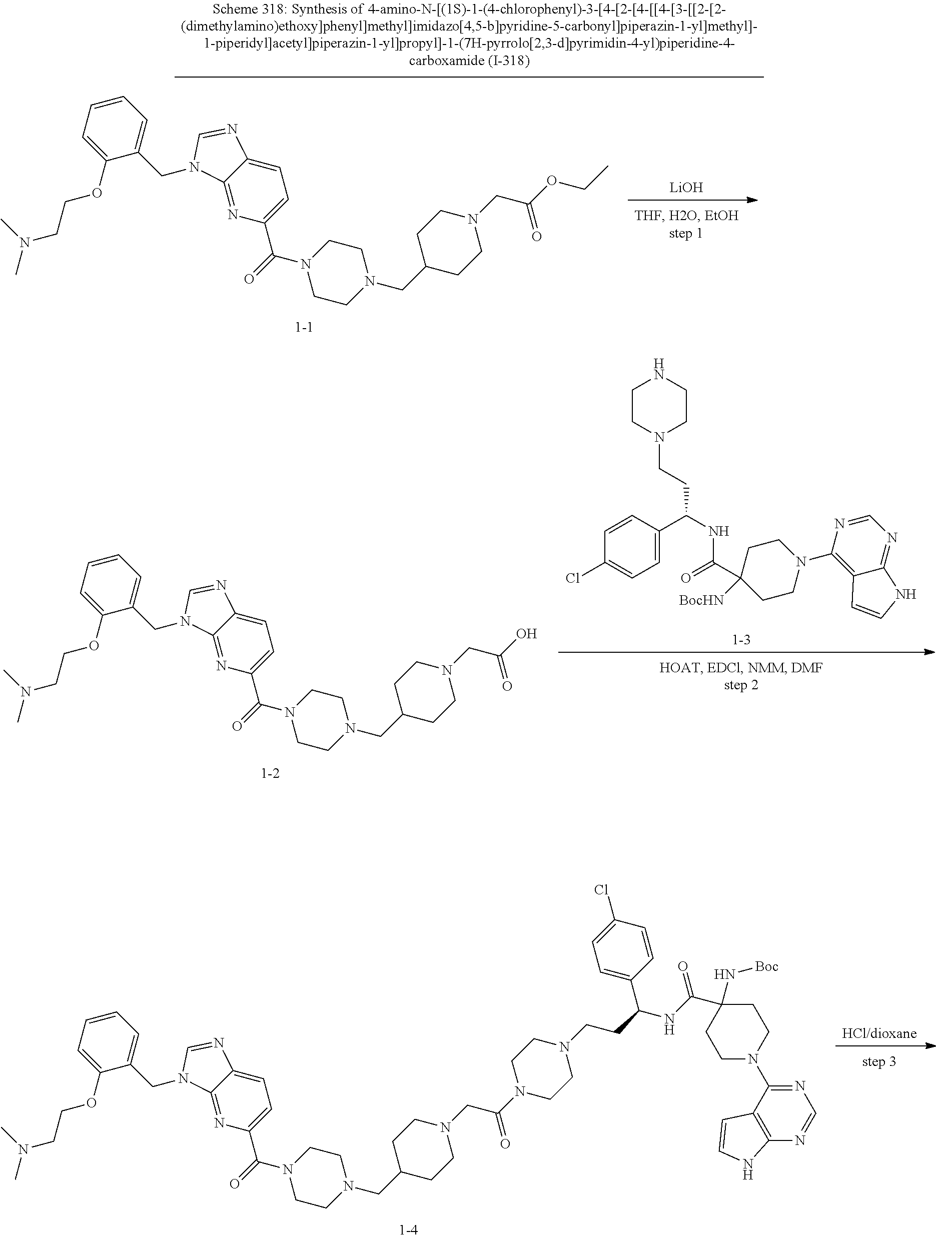

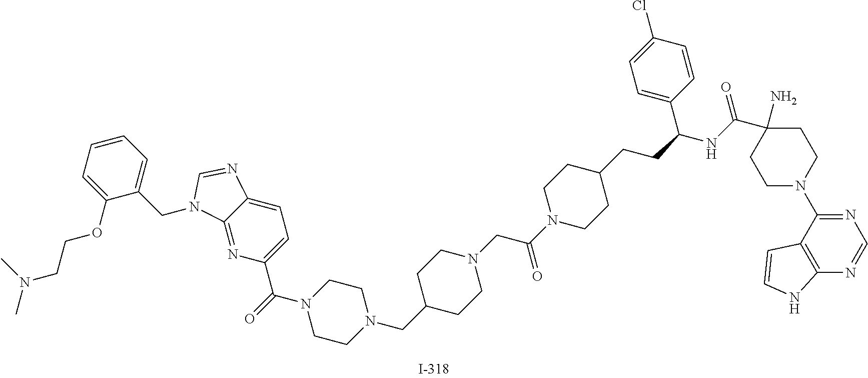 [SMILES: CN(C)CCOc1ccccc1Cn1cnc2ccc(C(=O)N3CCN(CC4CCN(CC(=O)N5CCC(CC[C@H](NC(=O)C6(N)CCN(c7ncnc8[nH]ccc78)CC6)c6ccc(Cl)cc6)CC5)CC4)CC3)nc21]

### Step 1: Synthesis of 1-2

Detailed Synthetic Procedure: To a solution of 1-1 (100 mg, 168.99 μmol, 1 eq) in THF (1 mL), H₂O (0.5 mL) and EtOH (0.5 mL) was added LiOH (8.09 mg, 337.98 μmol, 2 eq). The mixture was stirred at 25° C. for 1 hr. The mixture was poured into water 10 mL, then was added HCl (0.5 mol) to adjust pH≤7 and extracted with EA (10 mL*3). The combined organic layers were concentrated to give 1-2 (90 mg, 155.05 μmol, 91.75% yield, 97.11% purity) as a brown solid. LCMS: Rt=0.303 min, [M+H]⁺=564.4

### Step 2: Synthesis of 1-4

To a solution of 1-2 (80 mg, 141.92 μmol, 1 eq) and 1-3 (93.22 mg, 156.11 μmol, 1.1 eq) in DMF (1 mL) was added EDCI (81.62 mg, 425.77 μmol, 3 eq), HOAt (19.32 mg, 141.92 μmol, 19.85 μL, 1 eq) and NMM (143.55 mg, 1.42 mmol, 156.03 μL, 10 eq). The mixture was stirred at 25° C. for 1 hr. The mixture was poured into water 10 mL and extracted with EA (10 mL*3). The combined organic layers were concentrated to give a residue. The product was purified by prep-HPLC (column: Waters Xbridge 150*25 mm*5 um; mobile phase: [water (ammonia hydroxide v/v)-ACN]; gradient: 35%-65% B over min) and the eluent was concentrated to remove MeCN and then lyophilization. 1-4 (90 mg, 73.41 μmol, 51.72% yield, 93.21% purity) was obtained as a brown solid. LCMS: Rt=0.718 min, [M+H]⁺=1142.9

### Step 3: Synthesis of I-318

To a solution of 1-4 (80 mg, 70.00 μmol, 1 eq) in DCM (1 mL) was added HCl/dioxane (4 M, 1.33 mL, 76.19 eq). The mixture was stirred at 25° C. for 1 hr. The reaction mixture was concentrated under reduced pressure, then was added H₂O to lyophilization to give product. The product was without further purification. I-318 (68.53 mg, 63.50 μmol, 90.72% yield, 100% purity, HCl) was obtained as a yellow solid. LCMS: Rt=0.368 min, [M+H]⁺=1043.6. SFC: Rt=1.452 min, ee value=100%. ¹H NMR (400 MHZ, METHANOL-d4) δ=9.63-9.56 (m, 1H), 8.49-8.37 (m, 2H), 8.03 (d, J=8.4 Hz, 1H), 7.48-7.34 (m, 7H), 7.21 (d, J=8.4 Hz, 1H), 7.13-7.00 (m, 2H), 5.99-5.86 (m, 2H), 5.05-5.00 (m, 1H), 4.70-4.58 (m, 3H), 4.54-4.43 (m, 3H), 4.36-4.27 (m, 1H), 4.26-4.16 (m, 1H), 4.01-3.79 (m, 5H), 3.76-3.57 (m, 9H), 3.41-3.34 (m, 2H), 3.28-3.11 (m, 8H), 2.99 (s, 6H), 2.91-2.68 (m, 3H), 2.59-2.47 (m, 1H), 2.44-2.19 (m, 5H), 2.13-1.99 (m, 2H), 1.83-1.65 (m, 2H)

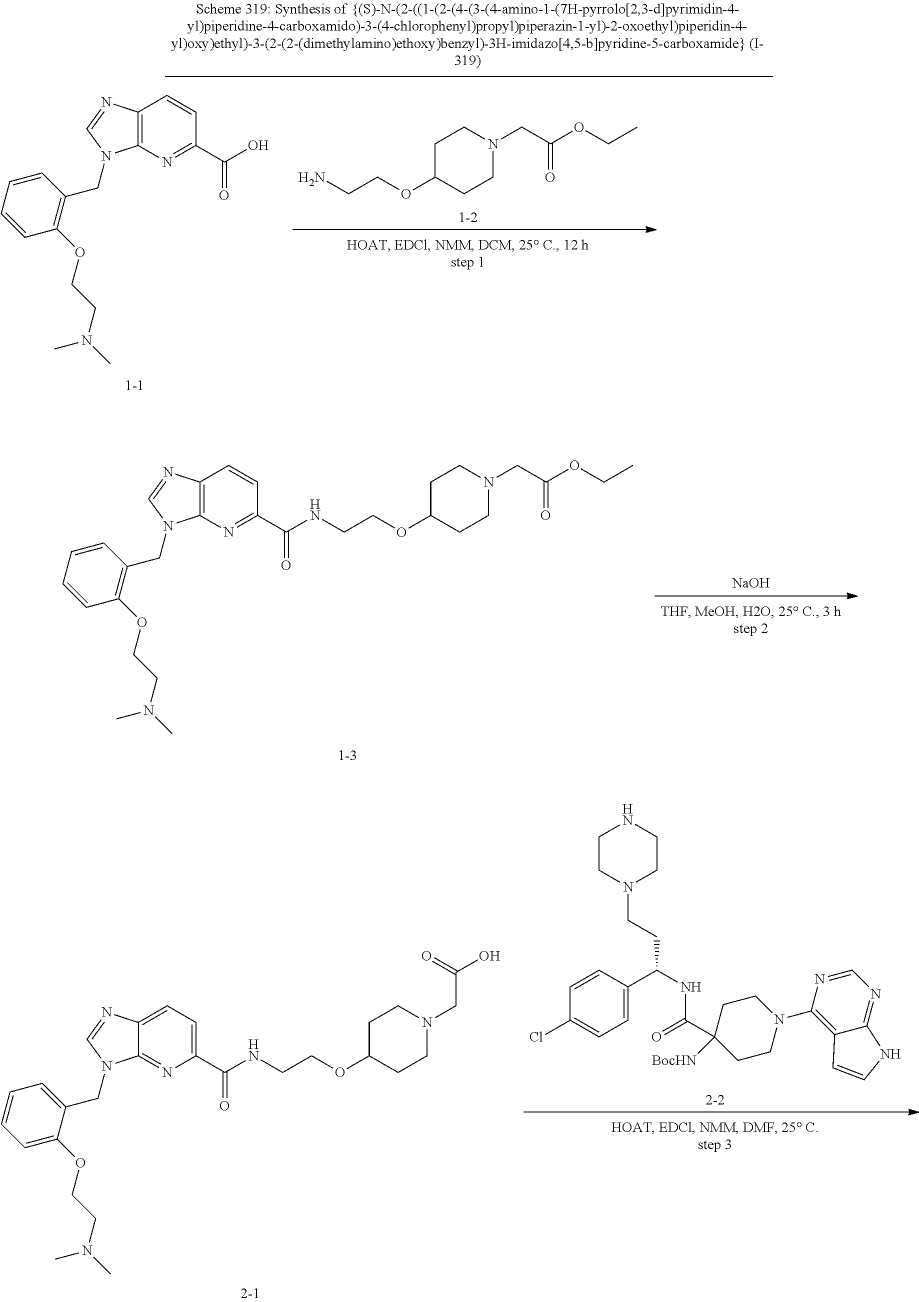

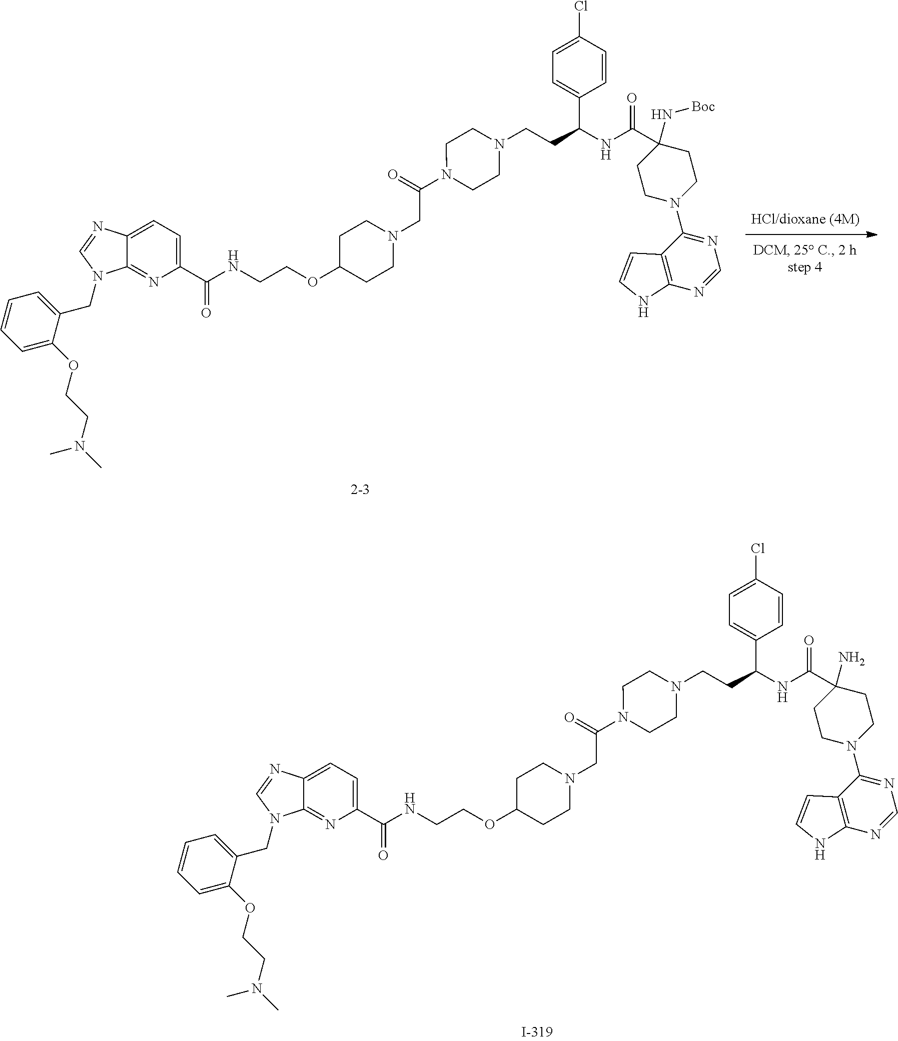 [SMILES: CN(C)CCOc1ccccc1Cn1cnc2ccc(C(=O)NCCOC3CCN(CC(=O)N4CCN(CC[C@H](NC(=O)C5(NC(=O)OC(C)(C)C)CCN(c6ncnc7[nH]ccc67)CC5)c5ccc(Cl)cc5)CC4)CC3)nc21 ; "HCl/dioxane (4M)" ; CN(C)CCOc1ccccc1Cn1cnc2ccc(C(=O)NCCOC3CCN(CC(=O)N4CCN(CC[C@H](NC(=O)C5(N)CCN(c6ncnc7[nH]ccc67)CC5)c5ccc(Cl)cc5)CC4)CC3)nc21]

To a mixture of 1-1 (1.1 g, 3.23 mmol, 1 eq) in DCM (11 mL) was added EDCI (1.24 g, 6.46 mmol, 2 eq), HOAt (439.87 mg, 3.23 mmol, 452.08 μL, 1 eq), NMM (2.29 g, 22.62 mmol, 2.49 mL, 7 eq) and 1-2 (1.03 g, 3.88 mmol, 1.2 eq, HCl). The mixture was stirred at 25° C. for 12 hr. The mixture was poured into water 20 mL and extracted with DCM (20 mL*3). The combined organic layers were dried over anhydrous Na₂SO₄, filtered and concentrated to give a residue. The residue was purified by flash silica gel chromatography (ISCO®; 40 g SepaFlash® Silica Flash Column, Eluent of 0˜10% Ethyl acetate/Petroleum ether gradient @ 40 mL/min) and the eluent was concentrated to give the product. 1-3 (1.06 g, 1.63 mmol, 50.45% yield, 85% purity) was obtained as yellow oil. 1-3 (30 mg, 46.14 μmol, 1 eq, 85% purity) was purified by prep-HPLC (column: YMC-Actus Triart C18 150*30 mm*7 um; mobile phase: [water (FA)-ACN]; gradient: 3%-33% B over 10 min) and the eluent was concentrated to remove MeCN and then lyophilization. 1-3 (20.18 mg, 33.03 μmol, 71.58% yield, 97.987% purity, FA) was obtained as colorless oil. LCMS: Retention time: 0.360 min, (M+H)+=553.4. ¹H NMR (400 MHZ, CHLOROFORM-d) δ=8.34 (s, 1H), 8.23 (d, J=8.4 Hz, 3H), 8.20-8.14 (m, 1H), 7.38-7.30 (m, 2H), 7.02-6.98 (m, 1H), 6.93 (d, J=8.4 Hz, 1H), 5.54 (s, 2H), 4.30-4.25 (m, 2H), 4.20-4.13 (m, 2H), 3.78-3.63 (m, 4H), 3.50-3.37 (m, 1H), 3.23 (s, 2H), 3.07-3.03 (m, 2H), 2.95-2.82 (m, 2H), 2.56 (s, 6H), 2.47-2.40 (m, 2H), 2.03-1.91 (m, 2H), 1.80-1.66 (m, 2H), 1.30-1.24 (m, 3H)

### Step 2: Synthesis of 2-1

To a solution of 1-3 (130 mg, 235.22 μmol, 1 eq, 85% purity) in THF (1 mL) and MeOH (0.5 mL) was added NaOH (37.63 mg, 940.90 μmol, 4 eq) in H2O (0.5 mL) and then the mixture was stirred at 25° C. for 3 h. The mixture was adjusted to pH=5˜6 with 1N HCl and then concentrated to give 2-1 (120 mg, crude) as a white solid. LCMS: Rt: 0.342 min, [M+H]⁺=525.3

### Step 3: Synthesis of 2-3

To a mixture of 2-1 (115.97 mg, 221.05 μmol, 1.1 eq), EDCI (115.57 mg, 602.86 μmol, 3 eq) and HOAt (27.35 mg, 200.95 μmol, 28.11 μL, 1 eq) in DCM (2 mL) was added NMM (101.63 mg, 1.00 mmol, 110.47 μL, 5 eq), the mixture was stirred at 25° C. for 10 min. Then 2-2 (120 mg, 200.95 μmol, 1 eq) was added. The mixture was stirred at 25° C. for 1 hr. The mixture was poured into water (5 mL) and extracted with DCM (5 mL*3). The combined organic layers were dried over anhydrous Na₂SO₄, filtered and concentrated to give 2-3 (200 mg, crude) as yellow solid. LCMS: Rt: 0.661 min, [1/2M+H]⁺=552.8

### Step 4: Synthesis of I-319

To a mixture of 2-3 (200 mg, 181.20 μmol, 1 eq) in DCM (2.5 mL) was added HCl/dioxane (4 M, 1 mL), the mixture was stirred at 25° C. for 2 hr. The reaction mixture was concentrated under reduced pressure to give the crude product. The residue was purified by prep-HPLC (column: Waters Xbridge 150*25 mm*5 um; mobile phase: [water (NH4HCO3)-ACN]; gradient: 20%-50% B over 9 min) and was lyophilized. I-319 (114 mg, 111.27 μmol, 61.40% yield, 97.956% purity) as yellow oil. LCMS: Rt: 0.604 min, [M+H]⁺=1003.7. SFC: Retention time: 0.954 min. ¹H NMR (400 MHZ, CHLOROFORM-d) δ=9.93 (br s, 1H), 8.61 (d, J=7.6 Hz, 1H), 8.56 (s, 1H), 8.38-8.31 (m, 1H), 8.30 (s, 1H), 8.22-8.09 (m, 2H), 7.44-7.38 (m, 1H), 7.34-7.28 (m, 3H), 7.19 (d, J=8.4 Hz, 2H), 7.05 (d, J=3.6 Hz, 1H), 6.96-6.85 (m, 2H), 6.49 (d, J=3.6 Hz, 1H), 5.47 (s, 2H), 5.06-5.00 (m, 1H), 4.62-4.40 (m, 2H), 4.11-4.07 (m, 2H), 3.78-3.46 (m, 10H), 3.45-3.35 (m, 1H), 3.10 (s, 2H), 2.82-2.73 (m, 2H), 2.72-2.68 (m, 2H), 2.41-2.15 (m, 16H), 2.04-1.85 (m, 4H), 1.68-1.47 (m, 6H)

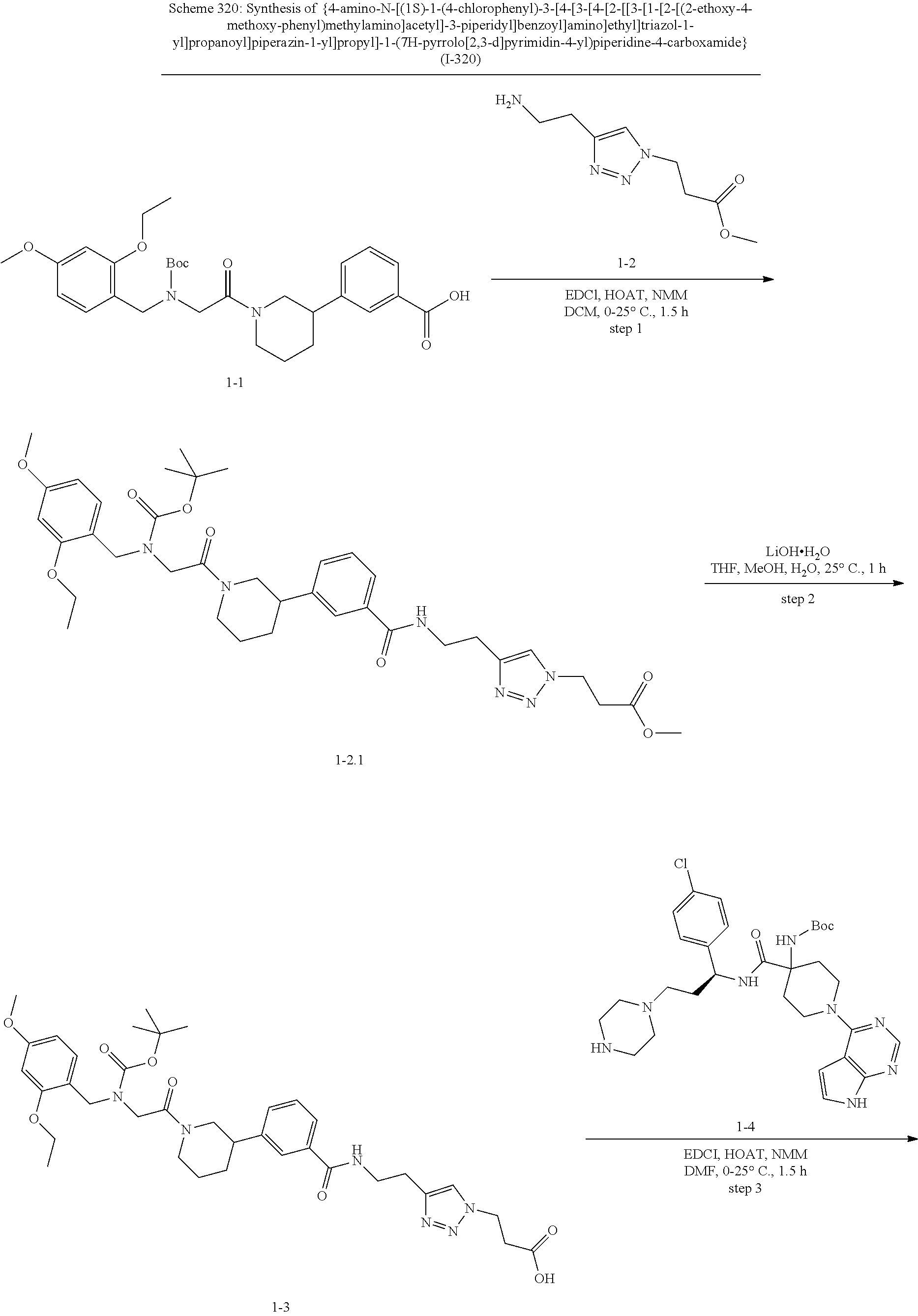 [SMILES: CCOc1cc(OC)ccc1CN(CC(=O)N1CCCC(c2cccc(C(=O)O)c2)C1)C(=O)OC(C)(C)C ; COC(=O)CCn1cc(CCN)nn1 ; CCOc1cc(OC)ccc1CN(CC(=O)N1CCCC(c2cccc(C(=O)NCCc3cn(CCC(=O)OC)nn3)c2)C1)C(=O)OC(C)(C)C ; CCOc1cc(OC)ccc1CN(CC(=O)N1CCCC(c2cccc(C(=O)NCCc3cn(CCC(=O)O)nn3)c2)C1)C(=O)OC(C)(C)C ; CC(C)(C)OC(=O)NC1(C(=O)N[C@@H](CCN2CCNCC2)c2ccc(Cl)cc2)CCN(c2ncnc3[nH]ccc23)CC1 ; "1-4"]

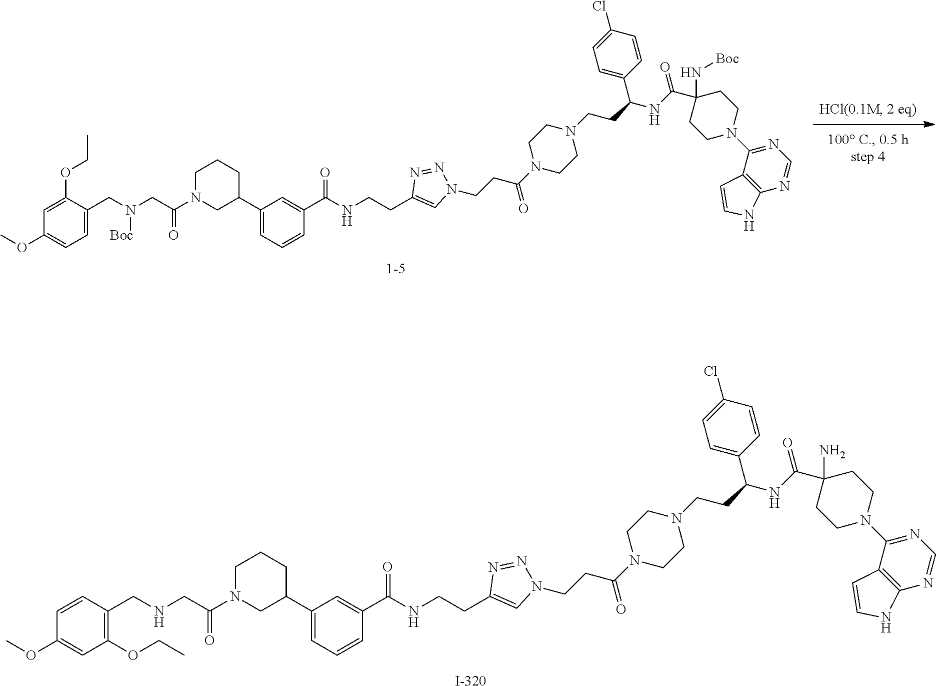 [SMILES: CCOc1cc(OC)ccc1CN(CC(=O)N1CCCC(c2cccc(C(=O)NCCc3cn(CCC(=O)N4CCN(CC[C@H](NC(=O)C5(NC(=O)OC(C)(C)C)CCN(c6ncnc7[nH]ccc67)CC5)c5ccc(Cl)cc5)CC4)nn3)c2)C1)C(=O)OC(C)(C)C ; "HCl(0.1M, 2 eq)" ; "1-5" ; CCOc1cc(OC)ccc1CNCC(=O)N1CCCC(c2cccc(C(=O)NCCc3cn(CCC(=O)N4CCN(CC[C@H](NC(=O)C5(N)CCN(c6ncnc7[nH]ccc67)CC5)c5ccc(Cl)cc5)CC4)nn3)c2)C1 ; "I-320"]

### Step 1: Synthesis of 1-2.1

To a solution of 1-1 (0.9 g, 1.71 mmol, 1 eq) in DCM (2 mL) was added HOBt (346.38 mg, 2.56 mmol, 1.5 eq), EDCI (982.85 mg, 5.13 mmol, 3 eq) and NMM (1.04 g, 10.25 mmol, 1.13 mL, 6 eq), then 1-2 (556.07 mg, 2.05 mmol, 1.2 eq, 2HCl) was added. The resulting solution was stirred at 20° C. for 1 hr under N₂. The reaction mixture was diluted with brine (20 mL), extracted with DCM (15 mL*2). The combined organic phase was washed with water (10 mL*2), dried over Na₂SO₄, filtered and concentrated under vacuum to give a residue. The residue was purified by reversed phase preparative HPLC (0.1% FA conditions) (300 g of XB—C18, 20-35 μm, 100 Å) Mobile phase: A for H2O (0.1% FA v/v) and B for acetonitrile; Gradient: B 0%-70% in 25 min: Flow rate: 100 ml/min; Column temperature: R.T. Wavelength: 220 nm/254 nm), after purification, and then concentrated to remove organic solvents. The residual aqueous solution was neutralized with NaHCO₃ to pH=8-9, extracted with EtOAc (30 mL*2). The combined organic phase was washed with brine (50 mL), dried over Na₂SO₄, filtered and concentrated to give 1-2.1 (0.75 g, 1.04 mmol, 60.75% yield, 97.850% purity) as a colorless gum. LCMS: Rt: 0.554 min, [M+H]⁺=707.4. 1H NMR (400 MHZ, DMSO-d₆) δ=8.64-8.52 (m, 1H), 7.96-7.87 (m, 1H), 7.79-7.63 (m, 2H), 7.50-7.35 (m, 2H), 7.14-7.01 (m, 1H), 6.62-6.42 (m, 2H), 4.60-4.52 (m, 2H), 4.51-4.40 (m, 1H), 4.38-4.17 (m, 2H), 4.07-3.97 (m, 3H), 3.86-3.68 (m, 4H), 3.63-3.56 (m, 3H), 3.56-3.46 (m, 2H), 3.15-3.00 (m, 1H), 2.99-2.93 (m, 2H), 2.92-2.84 (m, 2H), 2.77-2.55 (m, 2H), 2.01-1.89 (m, 1H), 1.85-1.68 (m, 2H), 1.57-1.26 (m, 14H).

### Step 2: Synthesis of 1-3

To a solution of Intermediated 1-2.1 (0.130 g, 183.92 μmol, 1 eq) in THF (1 mL), MeOH (1 mL) and H₂O (1 mL) was added LiOH·H2O (23.15 mg, 551.76 μmol, 3 eq). The resulting solution was stirred at 20° C. for 1 hr. The reaction mixture was neutralized by HCl (0.1M) to pH=7-8, concentrated under vacuum to remove THF and MeOH, then lyophilized. The crude product was used in next step according to theory amount without further purification. 1-3 (0.127 g, crude) was obtained as a white solid. LCMS: Rt: 0.527 min, [M+H]′=693.5

### Step 3: Synthesis of 1-5

To a solution of 1-4 (0.1 g, 167.46 μmol, 1 eq) and 1-3 (127 mg, 183.31 μmol, 1.09 eq) in DMF (3 mL) was added HOAt (34.19 mg, 251.19 μmol, 35.14 μL, 1.5 eq), EDCI (96.31 mg, 502.39 μmol, 3 eq) and NMM (101.63 mg, 1.00 mmol, 110.47 μL, 6 eq) at 20° C., and stirred for 1 hr under N₂. The reaction mixture was quenched by addition brine (10 mL) and saturated NaHCO₃ aqueous solution (2 mL), then extracted with EA 10 mL (5 mL*2). The combined organic layers were washed with brine (5 mL*4), dried over Na₂SO₄, filtered and concentrated under reduced pressure to give a residue. The residue was purified by reversed phase preparative HPLC (0.1% HCl conditions) (105 g of XB—C18, 20-35 μm, 100 Å) Mobile phase: A for H2O (0.1% HCl v/v) and B for acetonitrile; Gradient: B 5%-70% in 25 min: Flow rate: 60 ml/min; Column temperature: R.T. Wavelength: 220 nm/254 nm), after purification, and then concentrated to remove organic solvents then it was lyophilized. 1-5 (0.16 g, 93.82 μmol, 56.03% yield, 76.723% purity; HCl) was obtained as a white solid. LCMS: Rt: 0.541 min, [M+H]⁺=1271.7

### Step 4: Synthesis of I-320

A suspension of 1-5 (0.137 g, 104.71 μmol, 1 eq, HCl) in HCl (0.1 M, 2.51 mL, 2.4 eq) was stirred at 100° C. for 0.5 h. The reaction mixture was purified directly without workup. The reaction mixture was neutralized with NH₄OH (˜0.5%) to pH=8-9, dissolved in MeOH, purified by reversed phase preparative HPLC (0.1% NH₄OH conditions) (105 g of XB—C18, 20-35 μm, 100 Å) Mobile phase: A for H2O (0.1% NH₄OH v/v) and B for acetonitrile; Gradient: B 5%-70% in 25 min; Flow rate: 60 ml/min; Column temperature: R.T. Wavelength: 220 nm/254 nm), after purification, and then concentrated to remove organic solvents then it was lyophilized. I-320 (33.95 mg, 31.68 μmol, 30.25% yield, 100% purity) was obtained as a white solid. LCMS: Rt: 0.620 min, [M+H]⁺=1071.8. SFC: Rt: peak1 3.076 min, peak 2:3.878 min, IK-3-EtOH+ACN (DEA)-60-5 MIN-3ML-35T, Method details: “Column: Chiralpak IK-3 50*4.6 mm I.D., 3 um Mobile phase: Phase A for CO₂, and Phase B for EtOH+ACN (0.05% DEA): Gradient elution: 60% EtOH+ACN (0.05% DEA) in CO₂, Flow rate: 3 mL/min: Detector: PDA; Column Temp: 35 C; Back Pressure: 100 Bar”. ¹H NMR (400 MHZ, DMSO-d₆) δ=11.64 (br s, 1H), 8.73 (br d, J=8.0 Hz, 1H), 8.58 (br d, J=4.9 Hz, 1H), 8.11 (s, 1H), 7.90 (s, 1H), 7.78-7.65 (m, 2H), 7.40 (br d, J=6.4 Hz, 2H), 7.37-7.29 (m, 4H), 7.20-7.12 (m, 2H), 6.63-6.39 (m, 3H), 4.91-4.77 (m, 1H), 4.58-4.32 (m, 5H), 4.00 (qd, J=7.1, 14.4 Hz, 2H), 3.85-3.69 (m, 4H), 3.65 (br d, J=9.5 Hz, 2H), 3.59-3.38 (m, 10H), 3.12-2.97 (m, 2H), 2.97-2.82 (m, 5H), 2.79-2.54 (m, 3H), 2.35-2.15 (m, 6H), 2.03-1.81 (m, 5H), 1.80-1.65 (m, 2H), 1.42 (br t, J=13.8 Hz, 3H), 1.31 (td, J=6.9, 18.5 Hz, 3H)

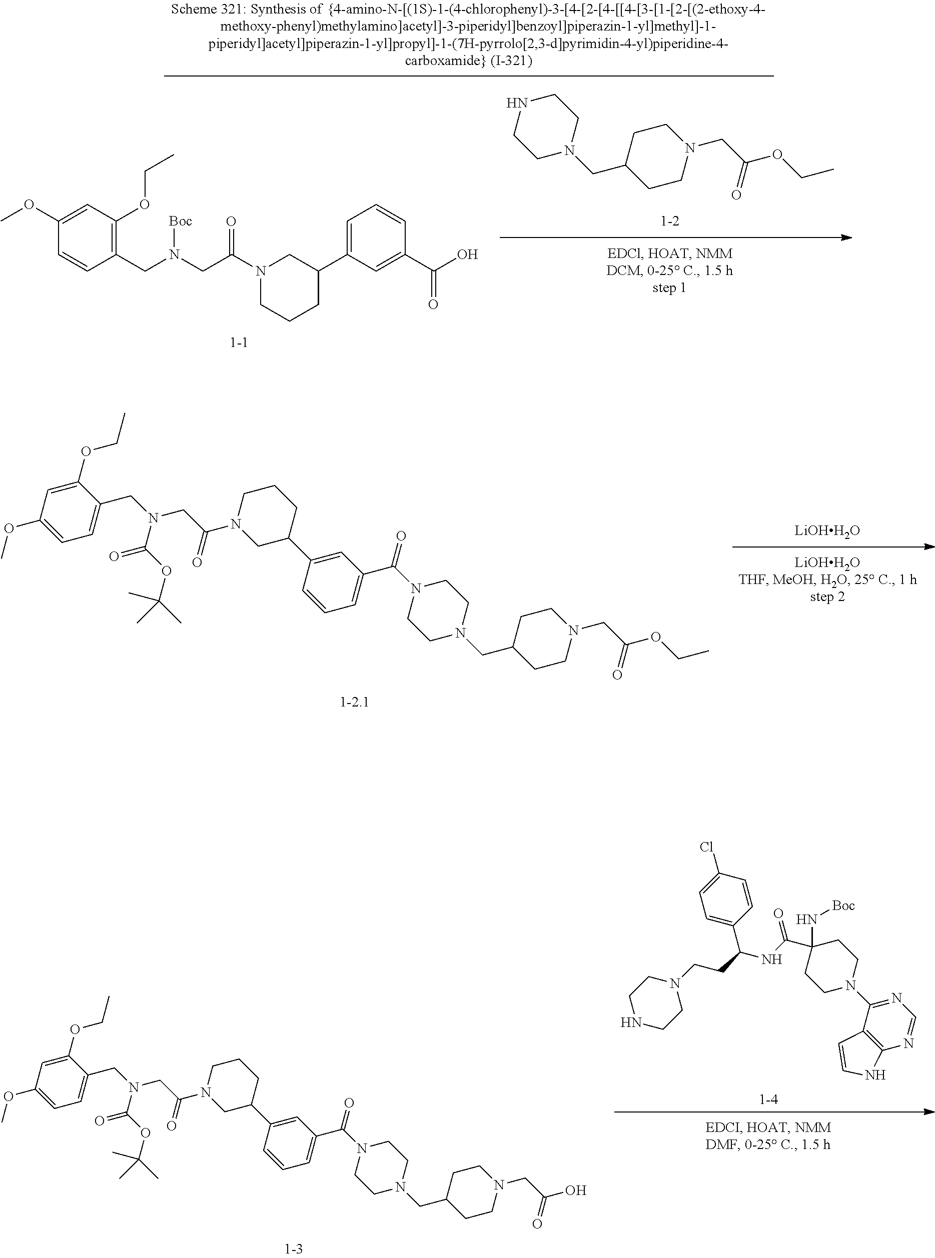

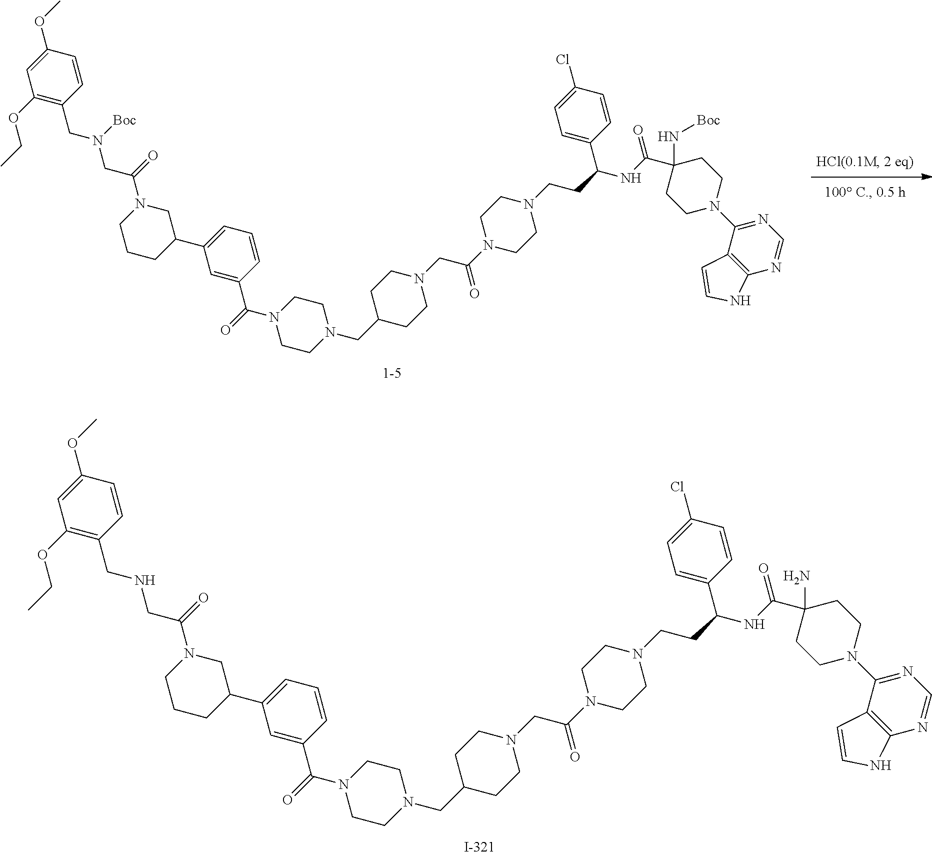 [SMILES: CCOc1cc(OC)ccc1CN(CC(=O)N1CCCC(c2cccc(C(=O)N3CCN(CC4CCN(CC(=O)N5CCN(CC[C@H](NC(=O)C6(NC(=O)OC(C)(C)C)CCN(c7ncnc8[nH]ccc78)CC6)c6ccc(Cl)cc6)CC5)CC4)CC3)c2)C1)C(=O)OC(C)(C)C ; "HCl(0.1M, 2 eq)" ; "1-5" ; CCOc1cc(OC)ccc1CNCC(=O)N1CCCC(c2cccc(C(=O)N3CCN(CC4CCN(CC(=O)N5CCN(CC[C@H](NC(=O)C6(N)CCN(c7ncnc8[nH]ccc78)CC6)c6ccc(Cl)cc6)CC5)CC4)CC3)c2)C1 ; "I-321"]

### Step 1: Synthesis of 1-2.1

To a solution of 1-1 (0.9 g, 1.71 mmol, 1 eq) in DCM (2 mL) was added HOBt (346.39 mg, 2.56 mmol, 1.5 eq), EDCI (982.86 mg, 5.13 mmol, 3 eq) and NMM (1.04 g, 10.25 mmol, 1.13 mL, 6 eq), then 1-2 (776.78 mg, 2.05 mmol, 1.2 eq, 3HCl) was added. The resulting solution was stirred at 20° C. for 1 h under N₂. The reaction mixture (together with page: ew37619-47) was diluted with brine (20 mL), extracted with DCM (15 mL*2). The combined organic phase was washed with water (10 mL*2), dried over Na₂SO₄, filtered and concentrated under vacuum to give a residue. The residue was purified by reversed phase preparative HPLC (0.1% FA conditions) (300 g of XB—C18, 20-35 μm, 100 Å) Mobile phase: A for H2O (0.1% FA v/v) and B for acetonitrile; Gradient: B 0%-70% in 25 min: Flow rate: 100 ml/min; Column temperature: R.T. Wavelength: 220 nm/254 nm), after purification, and then concentrated to remove organic solvents. The residual aqueous solution was neutralized with NaHCO₃ to pH=8-9, extracted with EtOAc (30 mL*2). The combined organic phase was washed with brine (50 mL), dried over Na₂SO₄, filtered and concentrated to give the product, then it was lyophilized. 1-2.1 (0.95 g, 1.19 mmol, 69.41% yield, 97.142% purity) was obtained as a pink gum. LCMS: Rt: 0.452 min, [M+H]′=778.6. ¹H NMR (400 MHZ, DMSO-d6) δ=7.44-7.31 (m, 2H), 7.30-7.20 (m, 2H), 7.12-7.01 (m, 1H), 6.57-6.43 (m, 2H), 4.49-4.34 (m, 1H), 4.34-4.16 (m, 2H), 4.14-3.85 (m, 6H), 3.84-3.50 (m, 6H), 3.32-3.24 (m, 2H), 3.21-3.14 (m, 2H), 3.12-2.93 (m, 1H), 2.84-2.76 (m, 2H), 2.74-2.55 (m, 2H), 2.47-2.23 (m, 4H), 2.19-2.04 (m, 4H), 1.99-1.84 (m, 1H), 1.83-1.69 (m, 2H), 1.68-1.58 (m, 2H), 1.53-1.27 (m, 14H), 1.22-1.15 (m, 3H), 1.15-1.02 (m, 2H).

### Step 2: Synthesis of 1-3

To a solution of 1-2.1 (0.143 g, 183.81 μmol, 1 eq) in THF (1 mL), MeOH (1 mL) and H₂O (1 mL) was added LiOH·H2O (23.14 mg, 551.42 μmol, 3 eq). The resulting solution was stirred at 20° C. for 1 hr. The reaction mixture was neutralized by HCl (0.1M) to pH=7-8, concentrated under vacuum to remove THF and MeOH, then lyophilized. The crude product was used in next step according to theory amount without further purification. 1-3 (0.138 g, crude) was obtained as a white solid. LCMS: Rt: 0.463 min, [M+H]⁺=750.6

### Step 3: Synthesis of 1-5

To a solution of 1-4 (0.1 g, 167.46 μmol, 1 eq) and 1-3 (0.138 g, 184.02 μmol, 1.1 eq) in DMF (3 mL) was added HOAt (34.19 mg, 251.19 μmol, 35.14 μL, 1.5 eq), EDCI (96.31 mg, 502.39 μmol, 3 eq) and NMM (101.63 mg, 1.00 mmol, 110.47 μL, 6 eq) at 20° C., then stirred for 16 hr under N₂. The reaction mixture was quenched by addition brine (10 mL) and saturated NaHCO₃ aqueous solution (2 mL), then extracted with EA 10 mL (5 mL*2). The combined organic layers were washed with brine (5 mL*4), dried over Na₂SO₄, filtered and concentrated under reduced pressure to give a residue. The residue was purified by reversed phase preparative HPLC (0.1% HCl conditions) (105 g of XB—C18, 20-35 μm, 100 Å) Mobile phase: A for H2O (0.1% HCl v/v) and B for acetonitrile; Gradient: B 5%-70% in 25 min: Flow rate: 60 ml/min; Column temperature: R.T. Wavelength: 220 nm/254 nm), after purification, and then concentrated to remove organic solvents then it was lyophilized. 1-5 (0.2 g, 120.86 μmol, 72.17% yield, 82.52% purity;
HCl) was obtained as a white solid. LCMS: Rt: 0.792 min, [M+H]⁺=1329.0

### Step 4: Synthesis of I-321

A solution of 1-5 (0.18 g, 154.47 μmol, 1 eq, HCl) in HCl (0.1 M, 3.09 mL, 2 eq) was stirred at 100° C. for 0.5 h under N₂. The reaction mixture was purified directly without workup. The reaction mixture was neutralized with NH₄OH (˜0.5%) to pH=8-9, dissolved in MeOH, purified by reversed phase preparative HPLC (0.1% NH₄OH conditions) (105 g of XB—C18, 20-35 μm, 100 Å) Mobile phase: A for H2O (0.1% NH₄OH v/v) and B for acetonitrile; Gradient: B 5%-70% in 25 min; Flow rate: 60 ml/min; Column temperature: R.T. Wavelength: 220 nm/254 nm), after purification, and then concentrated to remove organic solvents then it was lyophilized. I-321 (96.77 mg, 84.26 μmol, 54.55% yield, 98.296% purity) was obtained as a white solid. LCMS: Rt: 0.666 min, [M+H]⁺=1128.9. SFC: Rt: peak 1 4.252 min, peak 2:4.926 min, IK-3-MeOH+ACN (DEA)-60-9 MIN-3ML-35T, Method details: “Column: Chiralpak IK-3 50*4.6 mm I.D., 3 um Mobile phase: Phase A for CO₂, and Phase B for MeOH+ACN (0.05% DEA); Gradient elution: 60% MeOH+ACN (0.05% DEA)) in CO₂, Flow rate: 3 mL/min: Detector: PDA; Column Temp: 35 C; Back Pressure: 100 Bar”. ¹H NMR 400 MHZ, DMSO-d₆) δ=11.65 (br s, 1H), 8.73 (br d, J=8.0 Hz, 1H), 8.11 (s, 1H), 7.41-7.30 (m, 6H), 7.29-7.20 (m, 2H), 7.19-7.10 (m, 2H), 6.57 (dd, J=1.8, 3.5 Hz, 1H), 6.52-6.40 (m, 2H), 4.90-4.80 (m, 1H), 4.48-4.28 (m, 3H), 4.07-3.93 (m, 2H), 3.78 (br d, J=12.6 Hz, 1H), 3.72 (d, J=4.6 Hz, 3H), 3.66-3.48 (m, 8H), 3.48-3.36 (m, 4H), 3.31-3.27 (m, 2H), 3.13-2.93 (m, 3H), 2.83-2.53 (m, 5H), 2.43-2.08 (m, 14H), 2.03-1.79 (m, 7H), 1.78-1.60 (m, 4H), 1.53-1.37 (m, 4H), 1.31 (td, J=6.9, 16.0 Hz, 3H), 1.05 (br d, J=9.8 Hz, 2H).

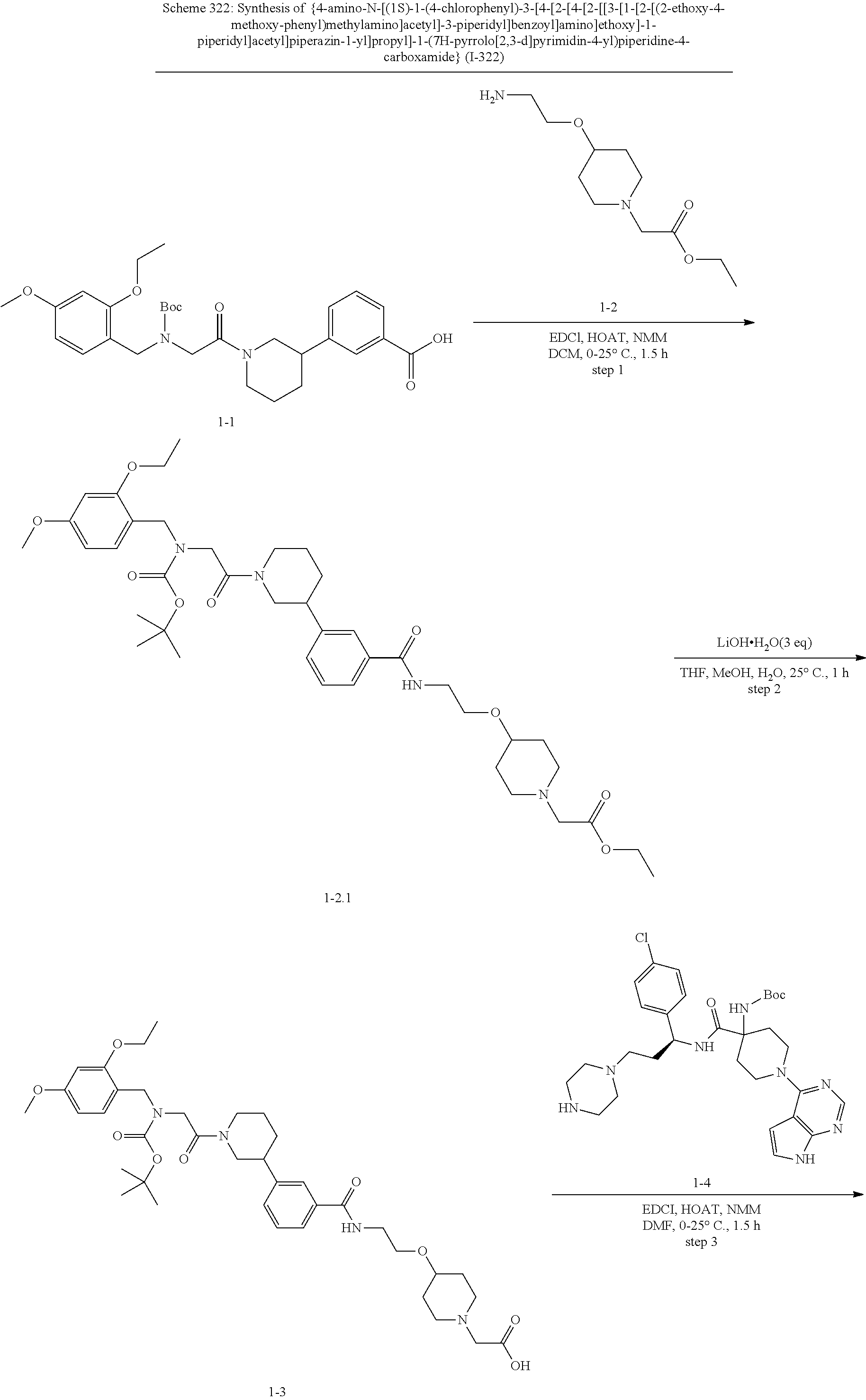

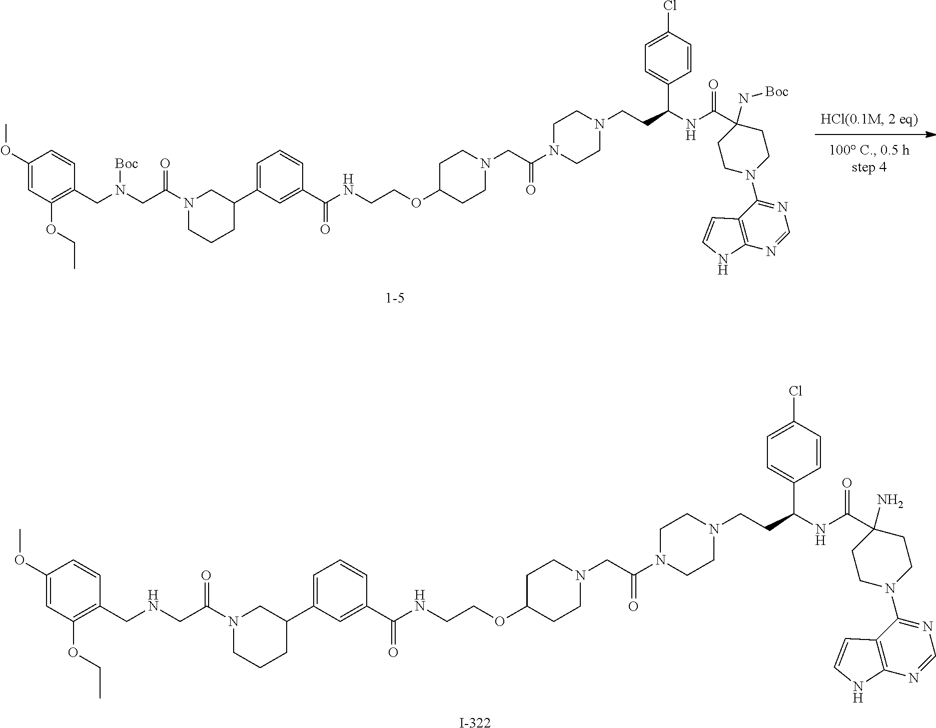 [SMILES: CCOc1cc(OC)ccc1CN(CC(=O)N1CCCC(c2cccc(C(=O)NCCOC3CCN(CC(=O)N4CCN(CC[C@H](NC(=O)C5(NC(=O)OC(C)(C)C)CCN(c6ncnc7[nH]ccc67)CC5)c5ccc(Cl)cc5)CC4)CC3)c2)C1)C(=O)OC(C)(C)C ; "HCl(0.1M, 2 eq)" ; CCOc1cc(OC)ccc1CNCC(=O)N1CCCC(c2cccc(C(=O)NCCOC3CCN(CC(=O)N4CCN(CC[C@H](NC(=O)C5(N)CCN(c6ncnc7[nH]ccc67)CC5)c5ccc(Cl)cc5)CC4)CC3)c2)C1 ; "I-322"]

### Step 1: Synthesis of 1-2.1

To a solution of 1-1 (0.9 g, 1.71 mmol, 1 eq) in DCM (2 mL) was added HOBt (346.38 mg, 2.56 mmol, 1.5 eq), EDCI (982.85 mg, 5.13 mmol, 3 eq) and NMM (1.04 g, 10.25 mmol, 1.13 mL, 6 eq). Then 1-2 (621.86 mg, 2.05 mmol, 1.2 eq, 2HCl) was added. The resulting solution was stirred at 20° C. for 1 h under N₂. The reaction mixture (together with page: ew37619-45) was diluted with brine (20 mL), extracted with DCM (15 mL*2). The combined organic phase was washed with water (10 mL*2), dried over Na₂SO₄, filtered and concentrated under vacuum to give a residue. The residue was purified by reversed phase preparative HPLC (0.1% FA conditions) (300 g of XB—C18, 20-35 μm, 100 Å) Mobile phase: A for H₂O (0.1% FA v/v) and B for acetonitrile; Gradient: B 0%-70% in 25 min: Flow rate: 100 ml/min; Column temperature: R.T. Wavelength: 220 nm/254 nm), after purification, and then concentrated to remove organic solvents. The residual aqueous solution was neutralized with NaHCO₃ to pH=8-9, extracted with EtOAc (30 mL*2). The combined organic phase was washed with brine (50 mL), dried over Na₂SO₄, filtered and concentrated to give the product, then it was lyophilized. 1-2.1 (1.07 g, 1.39 mmol, 81.43% yield, 96.107% purity) was obtained as an off white solid. LCMS: Rt: 0.520 min, [M+H]⁺=739.4. ¹H NMR (400 MHZ, DMSO-d₆) δ=8.59-8.39 (m, 1H), 7.81-7.64 (m, 2H), 7.49-7.34 (m, 2H), 7.18-6.97 (m, 1H), 6.60-6.42 (m, 2H), 4.53-4.40 (m, 1H), 4.38-4.17 (m, 2H), 4.14-3.91 (m, 6H), 3.85-3.67 (m, 4H), 3.58-3.49 (m, 2H), 3.44-3.36 (m, 2H), 3.31-3.25 (m, 1H), 3.20-3.14 (m, 2H), 3.13-2.94 (m, 1H), 2.79-2.58 (m, 4H), 2.31-2.20 (m, 2H), 2.00-1.89 (m, 1H), 1.87-1.69 (m, 4H), 1.47-1.27 (m, 15H), 1.21-1.14 (m, 3H).

### Step 2: Synthesis of 1-3

To a solution of 1-2.1 (0.136 g, 184.06 μmol, 1 eq) in THF (1 mL) and MeOH (1 mL) was added a solution of LiOH·H2O (23.17 mg, 552.17 μmol, 3 eq) in H2O (1 mL) dropwise at 20° C., and stirred at 20° C. for 1 hr under N₂. The reaction mixture was neutralized by HCl (0.1M) to pH=7-8, concentrated under vacuum to remove THF and MeOH, then lyophilized. The crude product was used in next step according to theory amount without further purification. 1-3 (0.130 g, crude) was obtained as a white solid. LCMS: Rt: 0.502 min, [M+H]⁺=711.5

### Step 3: Synthesis of 1-5

To a solution of 1-3 (0.130 g, 182.88 μmol, 1.09 eq) in DMF (3 mL) was added HOAt (45.59 mg, 334.92 μmol, 46.85 μL, 2 eq), EDCI (96.31 mg, 502.39 μmol, 3 eq) and NMM (101.63 mg, 1.00 mmol, 110.47 μL, 6 eq) under 20° C., then stirred at 20° C. for 1 h under N₂. The reaction mixture was quenched by addition brine (10 mL) and saturated NaHCO₃ aqueous solution (2 mL), then extracted with EA 10 mL (5 mL*2). The combined organic layers were washed with brine (5 mL*4), dried over Na₂SO₄, filtered and concentrated under reduced pressure to give a residue. The residue was purified by reversed phase preparative HPLC (0.1% HCl conditions) (105 g of XB—C18, 20-35 μm, 100 Å) Mobile phase: A for H2O (0.1% HCl v/v) and B for acetonitrile; Gradient: B 5%-70% in 25 min; Flow rate: 60 ml/min; Column temperature: R.T. Wavelength: 220 nm/254 nm), after purification, and then concentrated under vacuum to give a white solid. 1-5 (0.2 g, 150.78 μmol, 90.04% yield, 100% purity, HCl) was obtained as a white solid. LCMS: Rt: 0.753 min, [M+H]⁺=1290.0

### Step 4: Synthesis of I-322

To a solution of 1-5 (0.2 g, 146.74 μmol, 1 eq, 2HCl) in HCl (0.1 M, 2.93 mL, 2 eq) was stirred at 100° C. for 0.5 h under N₂. The reaction mixture was purified directly without workup. The reaction mixture was neutralized with NH₄OH (˜0.5%) to pH=8-9, dissolved in MeOH, purified by reversed phase preparative HPLC (0.1% NH₄OH conditions) (105 g of XB—C18, 20-35 μm, 100 Å) Mobile phase: A for H2O (0.1% NH4OHv/v) and B for acetonitrile; Gradient: B 5%-70% in 25 min; Flow rate: 60 ml/min; Column temperature: R.T. Wavelength: 220 nm/254 nm), after purification, and then concentrated to remove organic solvents then it was lyophilized. I-322 (104.90 mg, 95.96 μmol, 65.39% yield, 99.69% purity) was obtained as a white solid. LCMS: Rt: 0.655 min, [M+H]⁺=1089.8. SFC: Rt: peak 1 4.421 min, peak 2:5.794 min, IK-3-IPA+ACN (DEA)-60-7 MIN-3ML-35T, Method details: “Column: Chiralpak IK-3 50*4.6 mm I.D., 3 um Mobile phase: Phase A for CO₂, and Phase B for IPA+ACN (0.05% DEA); Gradient elution: 60% IPA+ACN (0.05% DEA) in CO₂, Flow rate: 3 mL/min; Detector: PDA; Column Temp: 35 C; Back Pressure: 100 Bar”. ¹H NMR 400 MHZ, DMSO-d₆) δ=11.66 (br s, 1H), 8.74 (br d, J=8.0 Hz, 1H), 8.55-8.42 (m, 1H), 8.12 (s, 1H), 7.81-7.65 (m, 2H), 7.46-7.30 (m, 6H), 7.22-7.08 (m, 2H), 6.63-6.38 (m, 3H), 4.92-4.80 (m, 1H), 4.52-4.33 (m, 3H), 4.00 (qd, J=7.0, 14.4 Hz, 2H), 3.86-3.75 (m, 1H), 3.73 (d, J=6.3 Hz, 3H), 3.67-3.49 (m, 8H), 3.48-3.35 (m, 7H), 3.12-2.96 (m, 3H), 2.79-2.55 (m, 5H), 2.44-2.15 (m, 7H), 2.10 (br t, J=9.6 Hz, 2H), 2.02-1.59 (m, 10H), 1.54-1.22 (m, 8H)

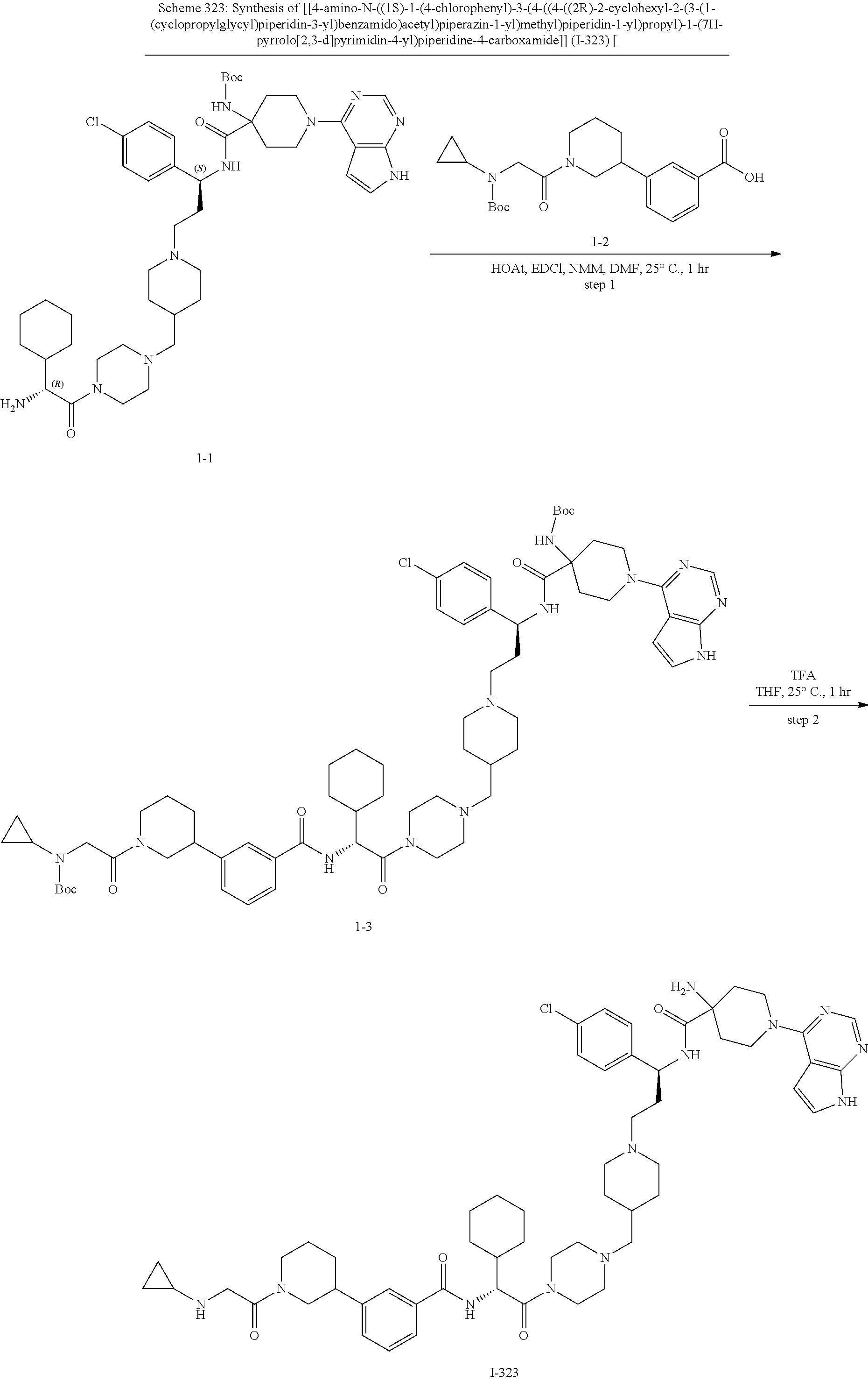

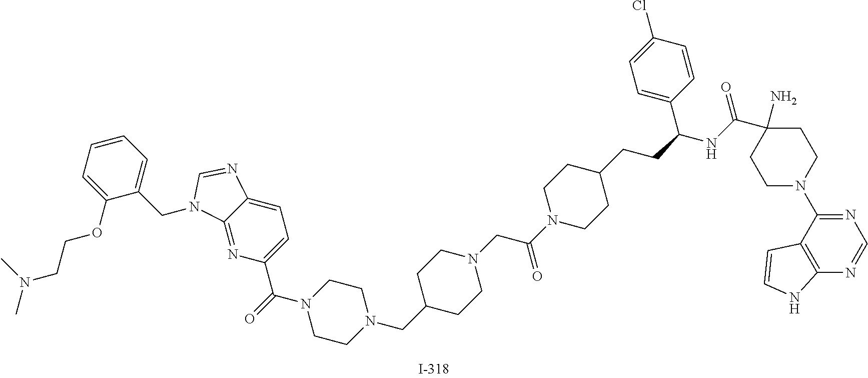 [SMILES: CN(C)CCOc1ccccc1Cn1cnc2ccc(C(=O)N3CCN(CC4CCN(CC(=O)N5CCC(CC[C@H](NC(=O)C6(N)CCN(c7ncnc8[nH]ccc78)CC6)c6ccc(Cl)cc6)CC5)CC4)CC3)nc21]

To a solution of tert-butyl 1-1 (50 mg, 59.99 μmol, 1.00 eq) in DMF (0.5 mL) was added HOAt (8.17 mg, 59.99 μmol, 8.39 μL, 1 eq), EDCI (34.50 mg, 179.96 μmol, 3 eq), NMM (30.34 mg, 299.94 μmol, 32.98 μL, 5 eq) and 1-2 (24.14 mg, 59.99 μmol, 1 eq). The mixture was stirred at 25° C. for 1 hr. The mixture was diluted with water (5 mL) and extract with EA (5 mL*3). The combined organic layers dried over sodium sulfate, filtered and concentrated under reduced pressure to give a residue. The residue was purified by reversed phase column (FA condition), the eluent was concentrated under reduced pressure to remove ACN and lyophilized to give 1-3 (50 mg, 38.77 μmol, 64.63% yield, 94.44% purity) as white solid. LCMS: Rt=0.399 min, [M/2+H]⁺=609.9. SFC: Rt=2.961 min

### Step 2: Synthesis of I-323

To a solution of 1-3 (40 mg, 32.84 μmol, 1 eq) in DCM (0.4 mL) was added TFA (0.08 mL). The mixture was stirred at 25° C. for 1 hr. The reaction mixture was concentrated under vacuo. The residue was purified by reversed phase column (TFA condition), the eluent was concentrated under reduced pressure to remove ACN and lyophilized to give I-323 (9.47 mg, 8.04 μmol, 24.48% yield, 96.071% purity, TFA) as a white solid. LCMS: Rt=0.276 min, [M+H]⁺=1017.5. SFC: Rt: 3.458 min, 4.435 min. 1H NMR (400 MHZ, METHANOL-d4) δ=8.13 (s, 1H), 7.79-7.66 (m, 2H), 7.51-7.42 (m, 2H), 7.37 (s, 4H), 7.13 (s, 1H), 6.62 (s, 1H), 4.94 (s, 1H), 4.57-4.45 (m, 3H), 3.83 (s, 2H), 3.79 (s, 2H), 3.71 (d, J=14.4 Hz, 2H), 3.65 (s, 2H), 3.56 (d, J=2.4 Hz, 1H), 3.44 (s, 1H), 3.28-3.11 (m, 2H), 2.94 (s, 1H), 2.90-2.81 (m, 2H), 2.73 (d, J=10.8 Hz, 3H), 2.49 (s, 2H), 2.41 (d, J=10.0 Hz, 2H), 2.26-2.16 (m, 6H), 2.08-1.85 (m, 8H), 1.79 (d, J=1.2 Hz, 2H), 1.73-1.56 (m, 6H), 1.45-1.36 (m, 2H), 1.33-1.20 (m, 4H), 1.16-1.06 (m, 2H), 0.63-0.52 (m, 4H). ¹⁹F NMR (376 MHz, METHANOL-d4) δ=−76.98

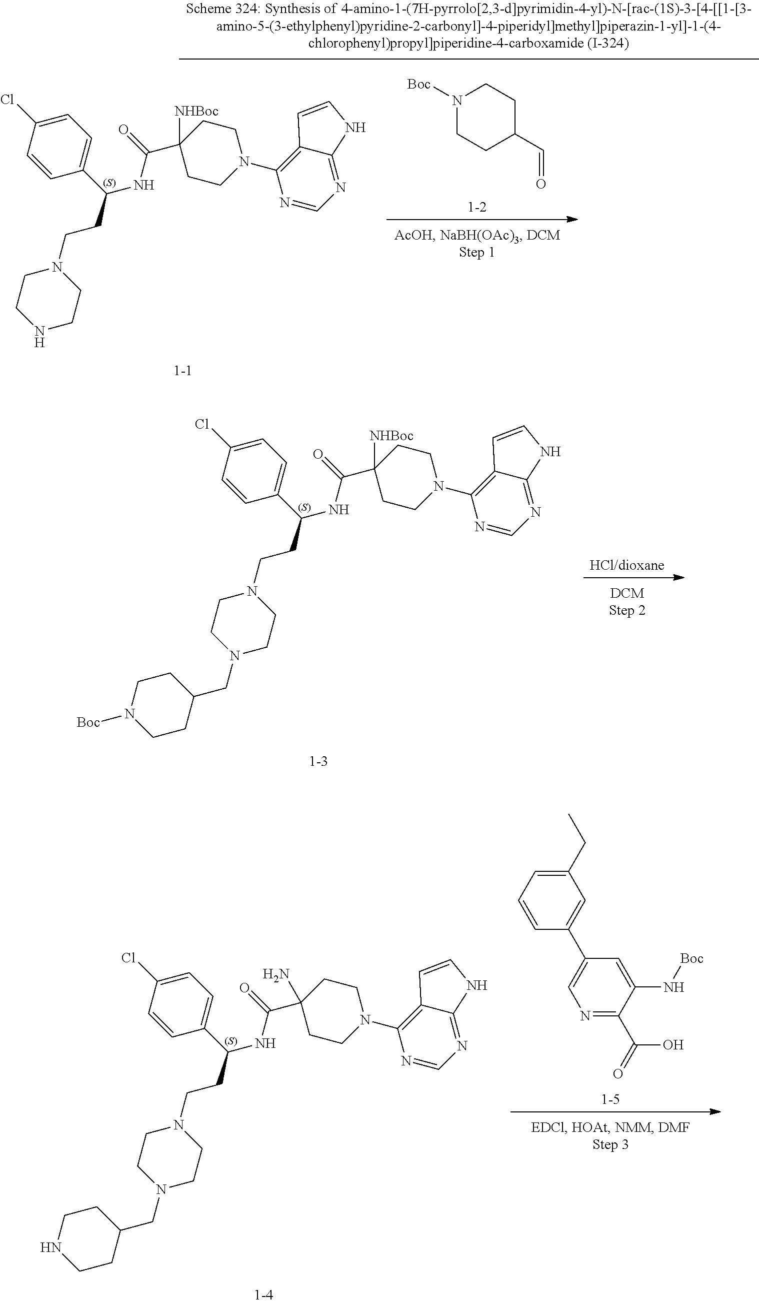

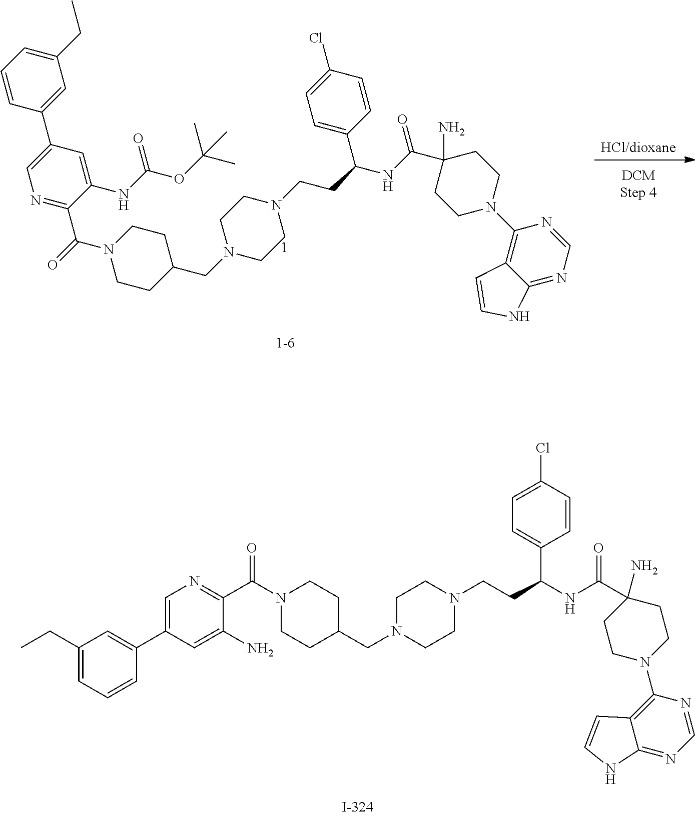 [SMILES: CCc1cccc(-c2cnc(C(=O)N3CCC(CN4CCN(CC[C@H](NC(=O)C5(N)CCN(c6ncnc7[nH]ccc67)CC5)c5ccc(Cl)cc5)CC4)CC3)c(NC(=O)OC(C)(C)C)c2)c1 ; "HCl/dioxane" ; CCc1cccc(-c2cnc(C(=O)N3CCC(CN4CCN(CC[C@H](NC(=O)C5(N)CCN(c6ncnc7[nH]ccc67)CC5)c5ccc(Cl)cc5)CC4)CC3)c(N)c2)c1]

### Step 1: Synthesis of 1-3.

A mixture of 1-1 (300 mg, 502.39 μmol, 1 eq), 1-2 (128.57 mg, 602.86 μmol, 1.2 eq) in DCM (3 mL) was added AcOH (60.34 mg, 1.00 mmol, 57.52 μL, 2 eq) and NaBH(OAc)₃ (532.38 mg, 2.51 mmol, 5 eq), then the mixture was stirred at 20° C. for 5 min. The reaction mixture was diluted with water (80 mL) and extracted with DCM (30 mL*2). The combined organic layers were concentrated under reduced pressure to give a residue. 1-3 (510 mg, crude) was obtained as a light yellow solid which was checked by SFC. LCMS: Retention time: 0.334 min, (M+H)=794.4. SFC: Retention time: 1.157 min

### Step 2: Synthesis of 1-4.

To a solution of 1-3 (100 mg, 125.88 μmol, 1 eq) in DCM (1 mL) was added HCl/dioxane (2 M, 314.69 μL, 5 eq). The mixture was stirred at 20° C. for 1 h. The reaction mixture was concentrated under reduced pressure to give a residue. The crude product was used into the next step without further purification. 1-4 (70 mg, 111.00 μmol, 88.18% yield, HCl) was obtained as a white solid which was checked by SFC. LCMS: Retention time: 0.201 min, (M+H)=594.4. SFC: Retention time: 1.797 min

### Step 3: Synthesis of 1-6.

A mixture of 1-4 (70 mg, 111.00 μmol, 1 eq, HCl), 1-5 (30.40 mg, 88.80 μmol, 0.8 eq) in DMF (1 mL) was added HOAt (15.11 mg, 111.00 μmol, 15.53 μL, 1 eq), EDCI (63.83 mg, 332.99 μmol, 3 eq) and NMM (56.13 mg, 554.98 μmol, 61.02 μL, 5 eq), then the mixture was stirred at 25° C. for 1 h. The reaction mixture was diluted with water 80 mL and extracted with DCM 60 mL (30 mL*2). The combined organic layers were concentrated under reduced pressure to give a residue. 1-6 (100 mg, 108.87 μmol, 98.08% yield) was obtained as a white solid. LCMS: Retention time: 0.370 min, (M+H)=918.5

### Step 4: Synthesis of I-324

To a solution of 1-6 (50 mg, 79.28 μmol, 1 eq, HCl) in DCM (1 mL) was added HCl/dioxane (2 M, 1 mL, 20.41 eq). Then the mixture was stirred at 20° C. for 1 hour. The reaction mixture was concentrated under reduced pressure to give a residue. The crude product was purified by prep-HPLC (column: Phenomenex luna C18 150*25 mm*10 um; mobile phase: [water (FA)-ACN]; gradient: 5%-35% B over 8 min). The eluent was concentrated under reduced pressure to remove ACN and then lyophilized to give the product. I-324 (14.24 mg, 16.47 μmol, 16.81% yield, 100% purity, FA) was obtained as a yellow solid. LCMS: Retention time: 0.306 min, (M+H)=818.5. SFC: Retention time: 0.799 min. ¹H NMR (400 MHz, DMSO-d₆) δ=11.66 (s, 1H), 8.67 (d, J=7.2 Hz, 1H), 8.21 (s, 1H), 8.12 (s, 1H), 8.07 (d, J=1.6 Hz, 1H), 7.47-7.23 (m, 8H), 7.16 (d, J=3.6 Hz, 1H), 6.58 (d, J=3.6 Hz, 1H), 5.49 (s, 1H), 4.82 (s, 1H), 4.55-4.34 (m, 3H), 3.78 (d, J=12.0 Hz, 1H), 3.63-3.44 (m, 2H), 3.07-2.94 (m, 1H), 2.84-2.62 (m, 3H), 2.36 (s, 6H), 2.20 (d, J=6.4 Hz, 2H), 2.16-1.68 (m, 9H), 1.61 (d, J=11.2 Hz, 1H), 1.52-1.44 (m, 2H), 1.25-1.20 (m, 3H), 1.17-0.99 (m, 2H)

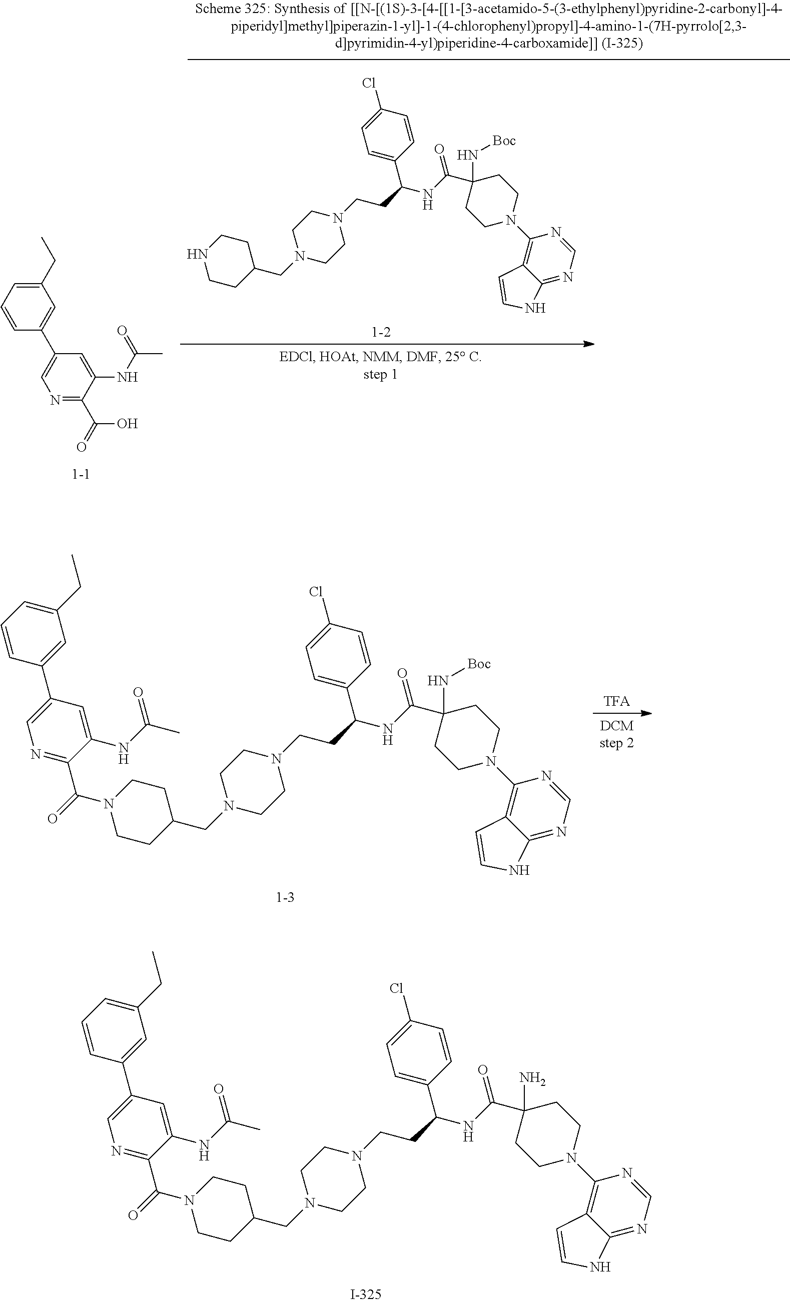

To a solution of 1-1 (360.35 mg, 1.27 mmol, 1.1 eq) in DMF (8 mL) was added EDCI (662.65 mg, 3.46 mmol, 3 eq), HOAt (156.83 mg, 1.15 mmol, 161.18 μL, 1 eq), NMM (582.74 mg, 5.76 mmol, 633.41 μL, 5 eq). The reaction mixture was stirred at 25° C. for 0.5 h, then 1-2 (800 mg, 1.15 mmol, 1 eq) was added to the reaction mixture and stirred at 25° C. for 12 h. The reaction mixture was purified by reverse phase column (330 g of XB—C18, 20-40 μm, 120 Å) Mobile phase: A for H₂O (0.1% FA v/v) and B for acetonitrile; Gradient: B 5%-100% in 15 min; Flow rate: 50 ml/min; Column temperature: R.T. Wavelength: 220 nm/254 nm). After purification, the eluent was concentrated to remove organic solvents and added a saturated solution of NaHCO₃ (20 mL) and then extracted with EA (20 mL×3). The combined organic layers were dried over sodium sulfate, filtered and concentrated under reduced pressure to give product. 1-3 (550 mg, 572.56 μmol, 49.69% yield) was obtained as a yellow solid. LCMS: Rt=0.479 min, [M+H]⁺=960.6. SFC: Rt=0.744 min. ¹H NMR (400 MHZ, CHLOROFORM-d) δ=9.71 (s, 1H), 8.96 (s, 1H), 8.55-8.41 (m, 2H), 8.32 (s, 1H), 7.49-7.37 (m, 3H), 7.29 (br s, 1H), 7.27-7.18 (m, 4H), 7.06 (br s, 1H), 6.53 (br s, 1H), 5.12-4.81 (m, 2H), 4.76-4.65 (m, 1H), 4.44-4.28 (m, 2H), 4.25-4.17 (m, 1H), 3.78-3.62 (m, 2H), 3.17-3.04 (m, 1H), 2.90-2.79 (m, 1H), 2.74 (q, J=7.6 Hz, 2H), 2.66-2.37 (m, 8H), 2.37-2.26 (m, 4H), 2.23 (s, 3H), 2.17 (d, J=3.6 Hz, 4H), 2.04-1.99 (m, 1H), 1.96-1.71 (m, 5H), 1.45 (s, 9H), 1.33-1.28 (m, 4H)

### Step 2: Synthesis of I-325

To a solution of 1-3 (550 mg, 572.56 μmol, 1 eq) in DCM (15 mL) was added TFA (5 mL). The reaction mixture was stirred at 20° C. for 0.5 h. The reaction mixture was concentrated to give crude product. The residue was dissolved with MeOH (1 mL) and adjust pH=7 by NH₃·H₂O (0.1%). The crude product was purified by reverse phase column (120 g of XB—C18, 20-40 μm, 120 Å) Mobile phase: A for H₂O (0.1% NH₃·H₂O v/v) and B for acetonitrile; Gradient: B 5%-100% in 30 min; Flow rate: 100 ml/min; Column temperature: R.T. Wavelength: 220 nm/254 nm). After purification, the eluent was concentrated to remove organic solvents and lyophilized to give product. I-325 (290 mg, 332.61 μmol, 58.09% yield, 98.691% purity) was obtained as a white solid. LCMS: Rt=1.886 min, [M+H]⁺=860.8. HPLC: Rt=7.745 min. SFC: Rt=0.924 min. ¹H NMR (400 MHZ, METHANOL-d4) δ=8.63 (d, J=1.8 Hz, 1H), 8.29 (d, J=1.8 Hz, 1H), 8.13 (s, 1H), 7.54-7.47 (m, 2H), 7.42 (t, J=7.6 Hz, 1H), 7.37-7.25 (m, 5H), 7.12 (d, J=3.6 Hz, 1H), 6.63 (d, J=3.6 Hz, 1H), 4.91-4.80 (m, 2H), 4.63-4.56 (m, 1H), 4.55-4.45 (m, 2H), 3.74-3.59 (m, 3H), 3.17-3.05 (m, 1H), 2.92-2.82 (m, 1H), 2.78-2.69 (m, 2H), 2.64-2.40 (m, 7H), 2.38-2.31 (m, 2H), 2.26-2.10 (m, 6H), 2.07-1.93 (m, 2H), 1.91-1.77 (m, 2H), 1.72-1.53 (m, 3H), 1.40-1.32 (m, 5H)

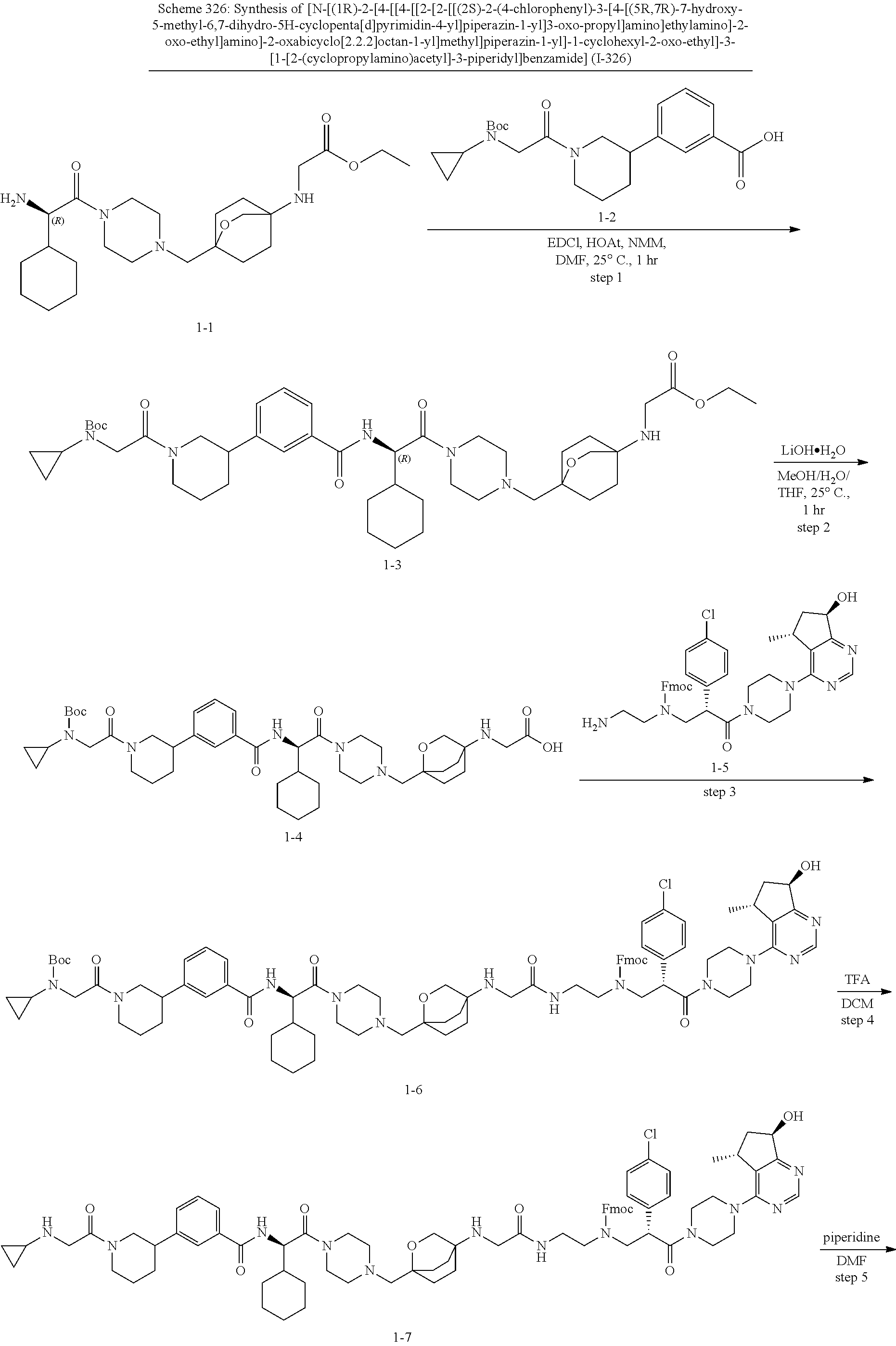

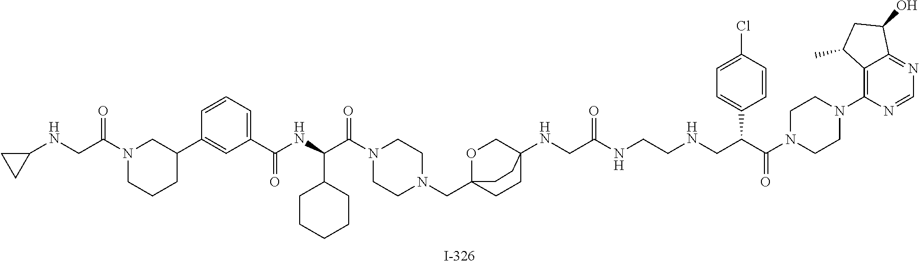 [SMILES: C[C@@H]1C[C@@H](O)c2ncnc(N3CCN(C(=O)[C@H](CNCCNC(=O)CNC45CCC(CN6CCN(C(=O)[C@H](NC(=O)c7cccc(C8CCCN(C(=O)CNC9CC9)C8)c7)C7CCCCC7)CC6)(CC4)OC5)c4ccc(Cl)cc4)CC3)c21]

To a solution of 1-1 (800 mg, 1.42 mmol, 1 eq, TFA) and 1-2 (570.25 mg, 1.42 mmol, 1 eq) in DMF (8 mL) was added EDCI (814.83 mg, 4.25 mmol, 3 eq), NMM (716.55 mg, 7.08 mmol, 778.85 μL, 5 eq) and HOAt (192.85 mg, 1.42 mmol, 198.20 μL, 1 eq). The mixture was stirred at 25° C. for 0.5 h. The reaction was washed with water and extracted with EA, then collected organic layer was concentrated to give crude product which was purified by prep-HPLC (0.1% FA condition) and the eluent was lyophilized to give 1-3 (800 mg, 938.83 μmol, 66.26% yield, 98% purity) was obtained as a white solid. SFC: Retention time: 2.057 min, 2.122 min. LCMS: Retention time: 0.373 min, [M+H]⁺=835.5

### Step 2: Synthesis of 1-4

To a solution of 1-3 (800 mg, 957.99 μmol, 1 eq) in MeOH (3 mL), THF (3 mL) and H₂O (3 mL) was added LiOH·H2O (120.60 mg, 2.87 mmol, 3 eq). The mixture was stirred at 25° C. for 0.5 h. Concentrated under reduced pressure to give a residue. The crude product was purified by prep-HPLC (0.1% FA condition) and the eluent was lyophilized to give 1-4 (530 mg, 656.73 μmol, 68.55% yield, 100% purity) was obtained as a white solid. LCMS: Retention time: 0.376 min, [M+H]⁺=807.5. LCMS: Retention time: 0.376 min, [M+H]⁺=807.5

### Step 3: Synthesis of 1-6

To a solution of 1-4 (53.36 mg, 74.35 μmol, 1.2 eq, HCl) in DMF (0.5 mL) was added EDCI (35.63 mg, 185.87 μmol, 3 eq), NMM (31.33 mg, 309.78 μmol, 34.06 μL, 5 eq), 1-5 (50 mg, 61.96 μmol, 1 eq) and HOAt (8.43 mg, 61.96 μmol, 8.67 μL, 1 eq). The mixture was stirred at 25° C. for 0.5 h. The reaction was washed with water and extracted with EA, then the collected organic layer was concentrated to give crude product. The crude product was purified by prep-HPLC (0.1% FA condition) to give 1-6 (120 mg, crude) as a white solid. LCMS: Retention time: 0.464 min, [M+H]⁺=1470.2

### Step 4: Synthesis of 1-7

To a solution of 1-6 (100 mg, 68.02 μmol, 1 eq) in DCM (1 mL) was added TFA (307.00 mg, 2.69 mmol, 200.00 μL, 39.59 eq). The mixture was stirred at 25° C. for 15 min. Concentrated under reduced pressure to give 1-7 (100 mg, crude, TFA) as a yellow oil. LCMS: Retention time: 0.392 min, (M+H)=1370.1

### Step 5: Synthesis of I-326

To a solution of 1-7 (100 mg, 67.38 μmol, 1 eq, TFA) in DMF (1 mL) was added piperidine (28.69 mg, 336.90 μmol, 33.27 μL, 5 eq). The mixture was stirred at 25° C. for 15 min. Without workup. The crude product was purified by prep-HPLC (neutral condition) and the eluent was lyophilized to give crude product. The crude product was second purified by prep-HPLC (column: CD02-Waters Xbidge BEH C18 150*25*10 um; mobile phase: [water (NH4HCO3)-ACN]; gradient: 31%-61% B over 10 min) and the eluent was lyophilized to give I-326 (20.34 mg, 17.39 μmol, 25.80% yield, 98.122% purity) was obtained as a white solid. SFC: Retention time: 2.683 min, 3.075 min. LCMS: Retention time: 0.648 min, [M/2+H]⁺=574.8. ¹H NMR (400 MHZ, METHANOL-d4) δ=8.42 (s, 1H), 7.76 (s, 1H), 7.74-7.67 (m, 1H), 7.53-7.41 (m, 2H), 7.41-7.36 (m, 2H), 7.36-7.30 (m, 2H), 4.97 (t, J=6.8 Hz, 1H), 4.90 (s, 2H), 4.63-4.53 (m, 1H), 4.22-4.18 (m, 1H), 3.96-3.85 (m, 2H), 3.83-3.67 (m, 5H), 3.63 (s, 3H), 3.60 (d, J=2.8 Hz, 2H), 3.58-3.44 (m, 5H), 3.37-3.33 (m, 2H), 3.30-3.22 (m, 2H), 3.21-3.09 (m, 3H), 2.88-2.68 (m, 5H). 2.65-2.46 (m, 4H), 2.31-2.22 (m, 3H), 2.17-2.09 (m, 1H), 2.08-2.02 (m, 2H), 2.01-1.93 (m, 2H), 1.93-1.82 (m, 4H), 1.75 (d, J=10.8 Hz, 3H), 1.69-1.59 (m, 6H), 1.40-1.02 (m, 9H), 0.49-0.47 (m 2H), 0.44-0.37 (m, 2H)

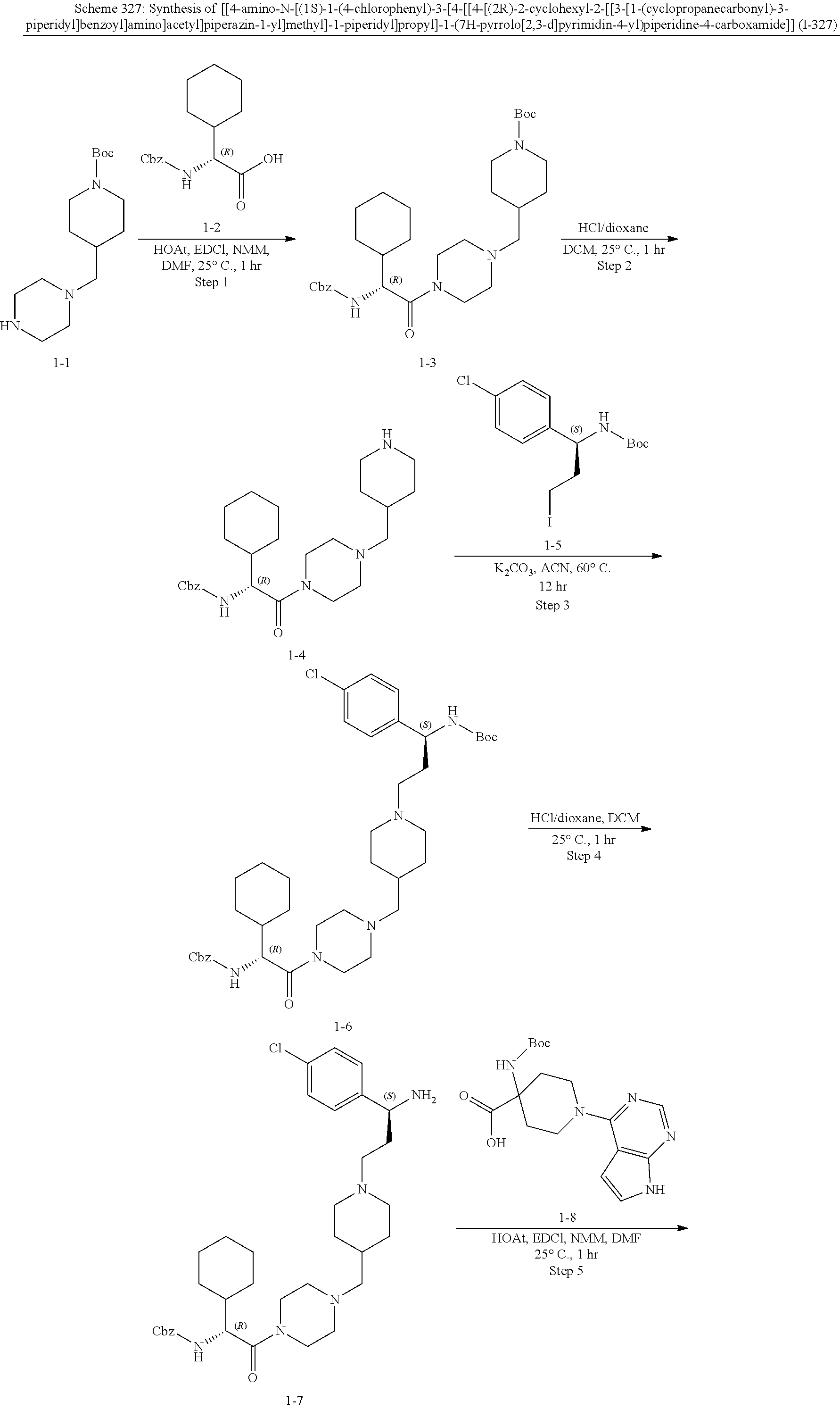

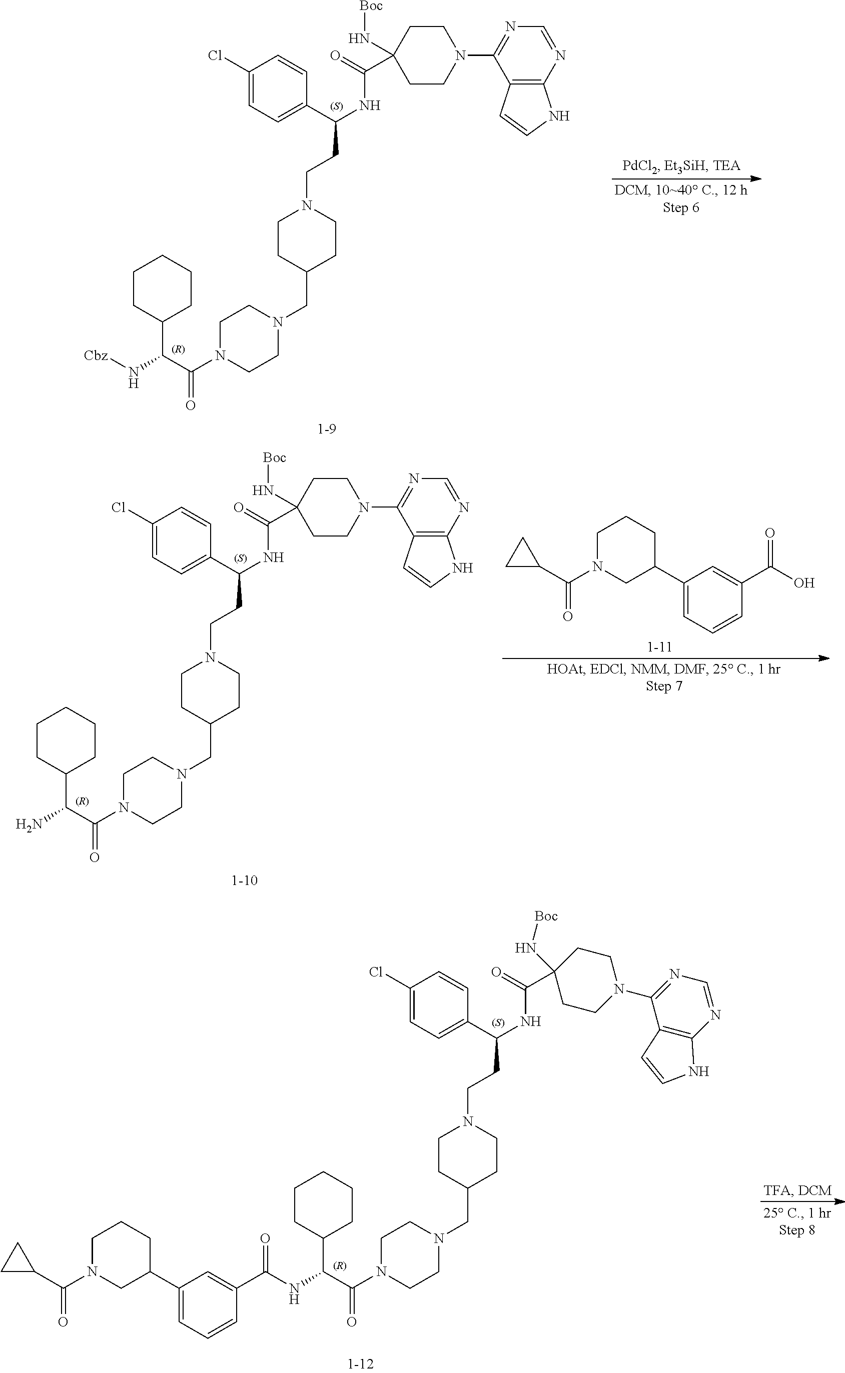

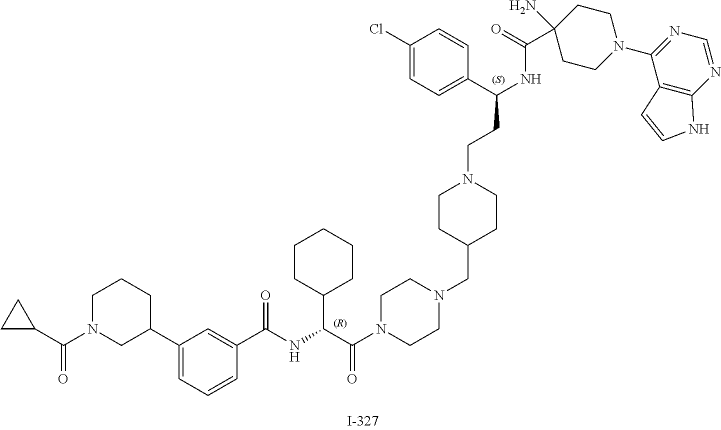

To a solution of 1-2 (257.00 mg, 882.12 μmol, 1 eq) in DMF (3 mL) was added EDCI (507.31 mg, 2.65 mmol, 3 eq), HOAt (120.07 mg, 882.12 μmol, 123.40 μL, 1 eq), NMM (446.12 mg, 4.41 mmol, 484.91 μL, 5 eq) and 1-1 (250 mg, 882.12 μmol, 1 eq). The mixture was stirred at 25° C. for 1 hr. The reaction mixture was diluted with H₂O (10 mL) and extracted with EA (30 mL, 10 mL*3). The combined organic layers were washed with brine (30 mL), dried over Na₂SO₄, filtered and concentrated under reduced pressure to give 1-3 (500 mg, crude) as yellow oil. LCMS: Retention time: 0.388 min, [M+H]⁺=557.3

### Step 2; Synthesis of 1-4

To a solution of 1-3 (500 mg, 898.09 μmol, 1 eq) in DCM (1 mL) was added HCl/dioxane (2 M, 5 mL). The mixture was stirred at 25° C. for 1 hr. The mixture was concentrated under reduced pressure to give 1-4 (450 mg, crude, HCl) as a yellow solid. LCMS: Retention time: 0.280 min, [M+H]⁺=457.1

### Step 3: Synthesis of 1-6

To a solution of 1-4 (450 mg, 912.63 μmol, 1 eq, HCl) in ACN (6 mL) was added 1-5 (361.09 mg, 912.63 μmol, 1 eq) and K₂CO₃ (378.39 mg, 2.74 mmol, 3 eq). The mixture was stirred at 60° C. for 12 hr. The mixture was filtered and concentrated under vacuum. The residue was purified by flash silica gel chromatography (ISCO®; 20 g SepaFlash® Silica Flash Column, Eluent of 0˜8% DCM/MeOH gradient (@ 50 mL/min). The eluent was concentrated under reduced pressure to give 1-6 (370 mg, 500.06 μmol, 54.79% yield, 97.9% purity) as a yellow solid. LCMS: Retention time: 0.373 min, [M+H]⁺=724.4

### Step 4: Synthesis of 1-7

To a solution of 1-6 (370 mg, 510.79 μmol, 1 eq) in DCM (1 mL) was added HCl/dioxane (2 M, 4 mL, 15.66 eq), the mixture was stirred at 25° C. for 1 hr. The mixture was concentrated under reduced pressure to give crude product. The crude product was purified by reversed phase column (0.1% HCl). The eluent was concentrated under reduced pressure to remove ACN and then lyophilized to give 1-7 (130 mg, 196.76 μmol, 38.52% yield, 100% purity; HCl) as a yellow solid. LCMS: Retention time: 0.289 min, [M+H]⁺=624.2

### Step 5: Synthesis of 1-9

To a solution of 1-7 (130 mg, 196.76 μmol, 1 eq, HCl) and 1-8 (71.11 mg, 196.76 μmol, 1 eq) in DMF (1.5 mL) was added EDCI (113.16 mg, 590.27 μmol, 3 eq), HOAt (26.78 mg, 196.76 μmol, 27.52 μL, 1 eq) and NMM (99.51 mg, 983.78 μmol, 108.16 μL, 5 eq). The mixture was stirred at 25° C. for 1 hr. The mixture was purified by reversed phase column (0.1% FA). The eluent was concentrated under reduced pressure to remove ACN and then lyophilized to give 1-9 (160 mg, 155.27 μmol, 78.91% yield, 93.9% purity) was obtained as a white solid. LCMS: Retention time: 0.347 min, [M+H]⁺=967.6. SFC: Retention time: 0.618 min

### Step 6: Synthesis of 1-10

To a solution of 1-9 (160 mg, 165.35 μmol, 1 eq) in DCM (2 mL) was added PdCl₂ (8.80 mg, 49.61 μmol, 0.3 eq) and TEA (33.46 mg, 330.70 μmol, 46.03 μL, 2 eq). The reaction mixture was degassed and purged with N₂ for 3 times. Then a solution of Et₃SiH (76.91 mg, 661.41 μmol, 105.64 μL, 4 eq) in DCM (1 mL) was added dropwise at 10° C. The resulting mixture was stirred at 25° C. for 2 hrs. The mixture was filtered and the filtrate was diluted with NaHCO₃ 5 mL and extracted with CH₂Cl₂ 10 mL (5 mL*2). The combined organic layers were dried over Na₂SO₄, filtered and concentrated under reduced pressure to give a residue. The residue was purified by reversed phase column (0.1% NH₃·H₂O). The eluent was concentrated under reduced pressure to remove ACN and then lyophilized to give 1-10 (60 mg, 71.99 μmol, 43.53% yield, 100% purity) as a white solid. LCMS: Retention time: 0.272 min, [M+H]⁺=833.7. SFC: Retention time: 1.343 min

### Step 7: Synthesis of 1-12

To a solution of 1-11 (21.64 mg, 79.18 μmol, 1.1 eq) in DMF (0.6 mL) was added EDCI (41.40 mg, 215.96 μmol, 3 eq), HOAt (9.80 mg, 71.99 μmol, 10.07 μL, 1 eq), 1-10 (60 mg, 71.99 μmol, 1 eq) and NMM (36.41 mg, 359.93 μmol, 39.57 μL, 5 eq). The mixture was stirred at 25° C. for 1 hr. The mixture was purified by reversed phase column (0.1% FA). The eluent was concentrated under reduced pressure to remove ACN and then lyophilized to give 1-12 (45 mg, 41.33 μmol, 57.41% yield) as a white solid. LCMS: Retention time: 0.357 min, [M+H]⁺=1088.4

### Step 8: Synthesis of I-327

To a solution of 1-12 (45 mg, 41.33 μmol, 1 eq) in DCM (0.5 mL) was added TFA (0.1 mL). The mixture was stirred at 25° C. for 1 hr. The reaction mixture was concentrated under reduced pressure to give a residue. The residue was purified by reversed phase column (0.1% TFA). The eluent was concentrated under reduced pressure to remove ACN and then lyophilized to give I-327 (6.63 mg, 6.01 μmol, 14.55% yield, 100% purity, TFA) as a white solid. LCMS: Retention time 0.323 min, [M+H]⁺=988.5. SFC: Retention time: 2.513 min, 2.894 min. HPLC: Retention time: 1.218 min. ¹H NMR (400 MHz, METHANOL-d4) δ=8.37 (s, 1H), 7.86-7.68 (m, 2H), 7.57-7.42 (m, 2H), 7.38 (s, 5H), 6.91 (s, 1H), 5.02 (s, 1H), 4.81 (s, 2H), 4.66-4.55 (m, 3H), 4.48-4.32 (m, 2H), 4.01-3.79 (m, 3H), 3.68-3.57 (m, 2H), 3.52-3.36 (m, 3H), 3.30-2.96 (m, 8H), 2.88-2.56 (m, 4H), 2.43-1.77 (m, 16H), 1.76-1.57 (m, 5H), 1.38-1.23 (m, 3H), 1.21-1.06 (m, 2H), 0.96-0.74 (m, 4H). ¹⁹F NMR (377 MHz, METHANOL-d4) δ=−77.007 (TFA)

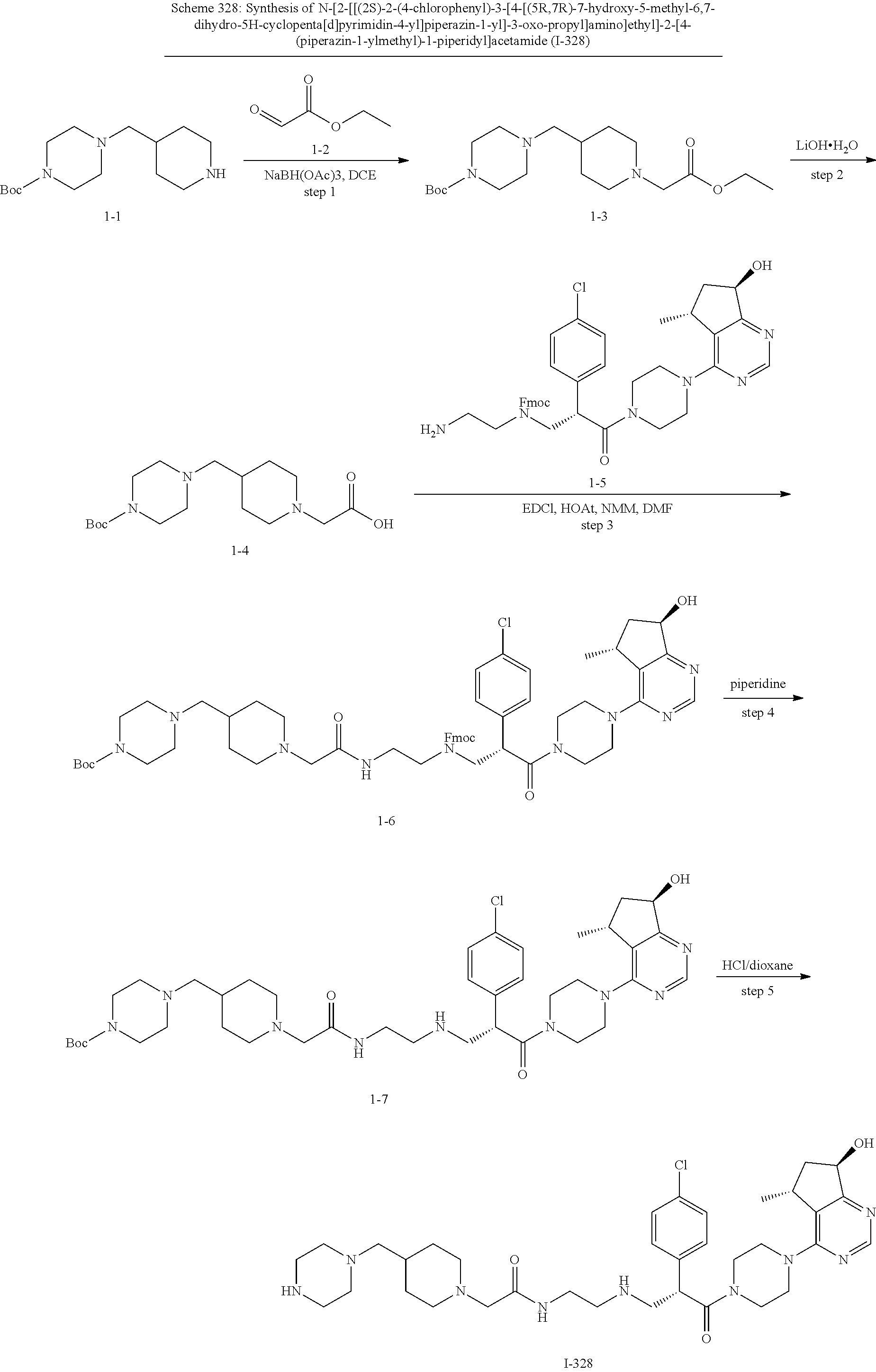

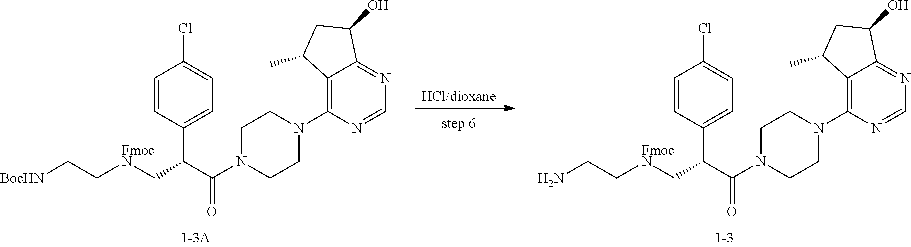

To a solution of 1-1 (30 g, 105.85 mmol, 1 eq) in DCE (300 mL) was added 4 A MS (3 g, 105.85 mmol, 1 eq). The mixture was stirred at 25° C. for 0.25 h. Then 1-2 (32.42 g, 158.78 mmol, 1.5 eq) was added. Then the mixture was stirred at 25° C. for 0.75 h. Then NaBH(OAc)₃ (22.43 g, 105.85 mmol, 1 eq) was added. The mixture was stirred at 25° C. for 2 h. The reaction mixture was quenched slowly by H₂O 600 mL, extracted with DCM 300 mL (100 mL*3). The combined organic layers were dried over Na₂SO₄, filtered and concentrated under reduced pressure to give a residue. The residue was purified by flash silica gel chromatography (ISCO®; 330 g SepaFlash® Silica Flash Column, Eluent of 0˜10% DCM/Methanol (@ 100 mL/min), 1-3 (28 g, 75.78 mmol, 71.59% yield) was obtained as a yellow oil. ¹H NMR (400 MHZ, CHLOROFORM-d) δ=4.25-4.02 (m, 3H), 3.40-3.30 (m, 4H), 3.15 (s, 2H), 2.88 (br d, J=11.3 Hz, 2H), 2.27 (br t, J=4.8 Hz, 4H), 2.18-2.01 (m, 4H), 1.68 (br d, J=12.8 Hz, 2H), 1.40 (s, 9H), 1.33-1.26 (m, 2H), 1.25-1.20 (m, 3H)

### Step 2: Synthesis of 1-4

To a solution of 1-3 (0.5 g, 1.35 mmol, 1 eq) in THF (3 mL), H₂O (1.5 mL) and MeOH (1.5 mL) was added LiOH·H₂O (113.56 mg, 2.71 mmol, 2 eq). The mixture was stirred at 25° C. for 2 hr. The mixture was adjust to pH=5 with 2N HCl and concentrated under vacuum. The residue was used in next step without purification. 1-4 (0.5 g, crude) as yellow solid was obtained. LCMS: Rt=0.356 min, [M−H]⁺=340.3

### Step 3: Synthesis of 1-6

A mixture of 1-5 (90 mg, 125.40 μmol, 1 eq, HCl), 1-4 (64.23 mg, 188.11 μmol, 1.5 eq) and HOAt (17.07 mg, 125.40 μmol, 17.54 μL, 1 eq) in DMF (1 mL) was added EDCI (48.08 mg, 250.81 μmol, 2 eq) and NMM (63.42 mg, 627.02 μmol, 68.94 μL, 5 eq). The mixture was stirred at 25° C. for 16 hrs. This reaction was used in next step without work up. This reaction was used in next step without purification. 1-6 (125 mg, 124.42 μmol, 99.22% yield) as yellow liquid in DMF was obtained. LCMS: Rt=0.793 min, [M+H]⁺=1004.7

### Step 4: Synthesis of 1-7

A mixture of 1-6 (125 mg, 124.42 μmol, 1 eq) in piperidine (862.20 mg, 10.13 mmol, 1 mL, 81.38 eq) was stirred at 25° C. for 0.5 hr. The mixture was diluted with water (2 mL), extracted with EtOAc (2 mL*3). The organic layer was concentrated under vacuum. The residue was purified by Prep-HPLC (0.1% NH₃·H₂O condition) and dried by lyophilization. 1-7 (40 mg, 51.12 μmol, 41.09% yield) as a yellow solid was obtained. LCMS: Rt=0.630 min, [M+H]⁺=782.6

### Step 5: Synthesis of I-328

A mixture of 1-7 (40 mg, 51.12 μmol, 1 eq) in DCM (1 mL) was added HCl/dioxane (2 M, 1.00 mL, 39.12 eq) and stirred at 25° C. for 0.5 hr. The mixture was concentrated under vacuum and dried by lyophilization. The mixture was adjust to pH=8 with Na₂CO₃ solution and extracted with DCM/MeOH=5/1 (6 mL*4). The organic layer was dried with anhydrous Na₂SO₄, filtered and concentrated under vacuum. I-328 (26.74 mg, 38.01 μmol, 74.35% yield, 96.989% purity) as a white solid was obtained. LCMS: Rt=0.342 min, [M+H]⁺=682.51. SFC: Rt=1.651 min. ¹H NMR (400 MHz, METHANOL-d4) δ=8.45-8.38 (m, 1H), 7.42-7.28 (m, 4H), 4.98-4.95 (m, 1H), 4.19-4.17 (m, 1H), 3.94-3.84 (m, 1H), 3.81-3.67 (m, 2H), 3.66-3.43 (m, 5H), 3.30-3.22 (m, 2H), 2.97 (s, 2H), 2.96-2.92 (m, 3H), 2.83 (br d, J=3.2 Hz, 2H), 2.77-2.72 (m, 2H), 2.47 (br s, 3H), 2.21 (d, J=7.2 Hz, 2H), 2.17-2.00 (m, 4H), 1.89 (s, 3H), 1.78-1.70 (m, 2H), 1.55-1.54 (m, 1H), 1.35-1.20 (m, 4H), 1.18-1.06 (m, 3H)

### Step 6: Synthesis of 1-3

A mixture of 1-3A (0.1 g, 127.99 μmol, 1 eq) in DCM (1 mL) was added HCl/dioxane (4 M, 0.5 mL, 15.63 eq) and stirred at 25° C. for 0.5 hr. The mixture was concentrated under vacuum and dried by lyophilization. This step without purification. 1-3 (90 mg, crude, HCl) as yellow oil was obtained. LCMS: Rt=0.778 min, [M+H]⁺=681.4

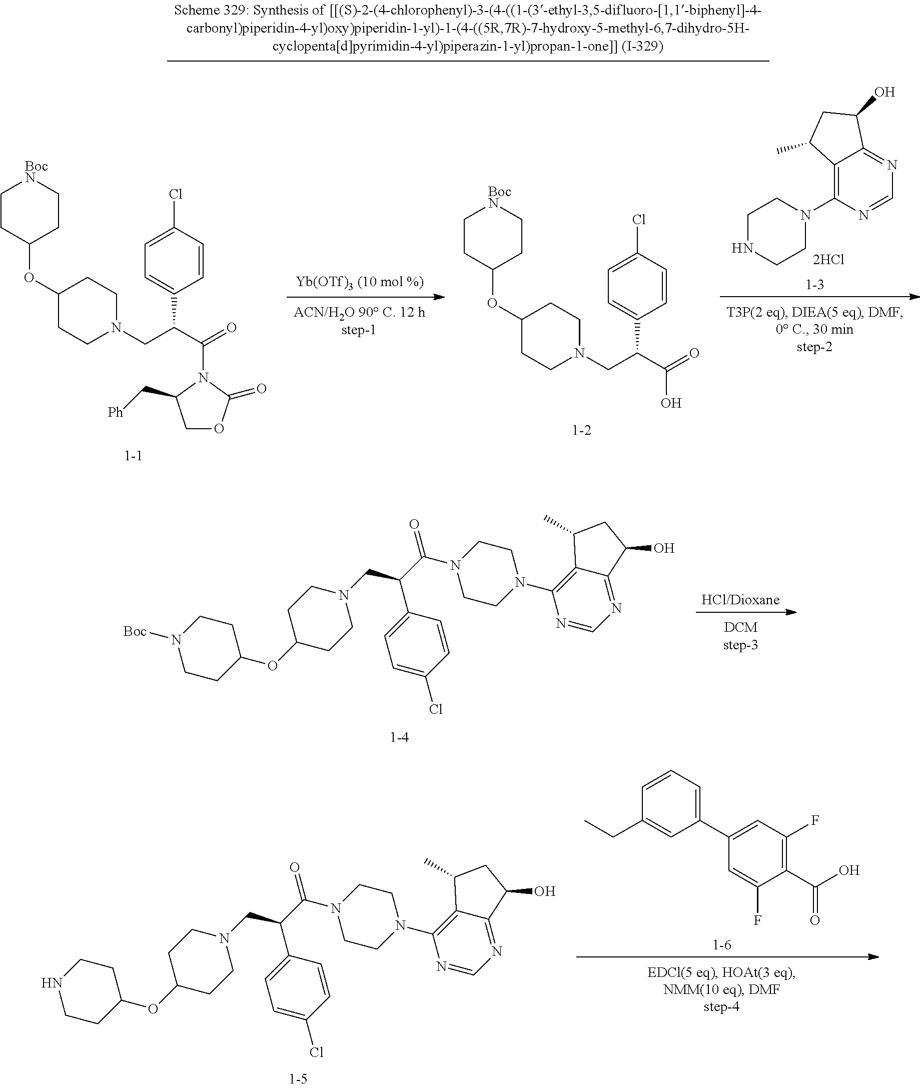

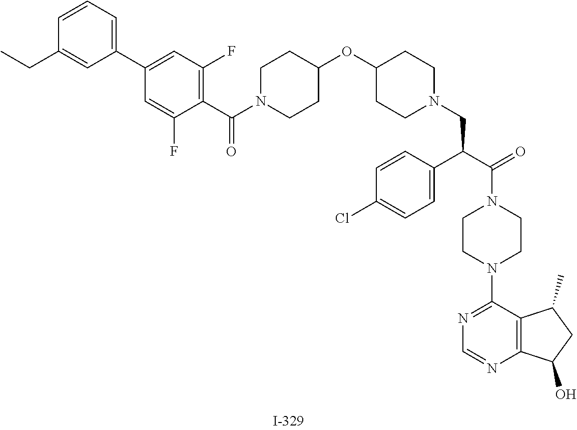 [SMILES: CCc1cccc(-c2cc(F)c(C(=O)N3CCC(OC4CCN(C[C@@H](C(=O)N5CCN(c6ncnc7c6[C@H](C)C[C@H]7O)CC5)c5ccc(Cl)cc5)CC4)CC3)c(F)c2)c1]

### Step 1: Synthesis of 1-2

To a solution of 1-1 (8 g, 12.78 mmol, 1 eq) in H2O (20 mL) and MeCN (80 mL) was added Yb(OTf)₃ (792.42 mg, 1.28 mmol, 0.1 eq). The mixture was stirred at 90° C. for 16 h. The mixture evaporated to give a residue. The residue was purified by Prep-HPLC (FA condition) to afford 1-2 (1.55 g, 3.28 mmol, 25.64% yield, 98.713% purity) as a white solid. LCMS: Rt=0.458 min, [M+H]⁺=467.3. ¹H NMR (400 MHz, DMSO-d₆) δ=7.40-7.35 (m, 2H), 7.34-7.29 (m, 2H), 3.85-3.81 (m, 1H), 3.64-3.55 (m, 5H), 3.07-2.98 (m, 3H), 2.81-2.74 (m, 1H), 2.44-2.35 (m, 1H), 2.31-2.21 (m, 1H), 1.85-1.68 (m, 4H), 1.48-1.23 (m, 14H).

### Step 2: Synthesis of 1-4

To a solution of 1-2 (800 mg, 1.71 mmol, 1 eq) and 1-3 (631.55 mg, 2.06 mmol, 1.2 eq, 2HCl) in DCM (10 mL) was added T4P (3.70 g, 5.14 mmol, 50% purity, 3 eq) and DIEA (1.11 g, 8.57 mmol, 1.49 mL, 5 eq). The mixture was stirred at 25° C. for 1 hr. The reaction mixture was filtered give a residue. The residue was purified by reverse phase (water (FA)-ACN). The eluent was concentrated under reduced pressure to remove ACN, then lyophilized to afford product. 1-4 (800 mg, 1.17 mmol, 68.35% yield) was obtained as a white solid. LCMS: Rt=0.441 min, [M+H]⁺=683.41.

### Step 3: Synthesis of 1-5

The mixture of 1-4 (800 mg, 1.17 mmol, 1 eq) in DCM (8 mL) and 2M HCl/dioxane (8 mL) was stirred at 25° C. for 1 hr. The reaction was concentrated under reduced pressure to give a residue. The crude product 1-5 (700 mg, 1.13 mmol, 96.49% yield, HCl) was a yellow solid, and it was used into the next step without further purification. LCMS: Retention time: 0.695 min, [M+H]⁺=583.3

### Step 4: Synthesis of I-329

To a solution of 1-5 (700 mg, 1.13 mmol, 1 eq, HCl) and 1-6 (237.02 mg, 903.77 μmol, 0.8 eq) in DCM (7 mL) was added NMM (571.35 mg, 5.65 mmol, 621.04 μL, 5 eq), HOAt (153.77 mg, 1.13 mmol, 158.03 μL, 1 eq) and EDCI (649.70 mg, 3.39 mmol, 3 eq). The mixture was stirred at 25° C. for 1 hr. The reaction was diluted with H₂O (5 mL), extracted with ethyl acetate 30 mL (10 mL*3), The organic phase was washed with saturated aqueous NaCl (5 mL). Then dried over Na₂SO₄, filtered and concentrated under reduced pressure to give a residue. The residue was purified by prep-HPLC (column: Phenomenex luna C18 150*25 mm*10 um; mobile phase: [water (FA)-ACN]; gradient: 15%-45% B over 15 min). The eluent was concentrated under reduced pressure to remove ACN, then lyophilized to afford product. I-329 (235 mg, 267.28 μmol, 23.66% yield, 99.34% purity, FA) was obtained as a white solid. LCMS: Retention time: 14.933 min, [M+H]⁺=827.2. SFC: Rt=1.112 min. ¹H NMR (400 MHZ, METHANOL-d4) δ=8.56-8.52 (m, 1H), 8.45 (d, J=1.2 Hz, 1H), 7.56-7.45 (m, 2H), 7.45-7.26 (m, 8H), 4.99 (t, J=6.8 Hz, 1H), 4.35-4.32 (m, 1H), 4.12-4.02 (m, 1H), 3.91-3.76 (m, 4H), 3.74-3.53 (m, 8H), 3.47-3.37 (m, 2H), 3.26-3.18 (m, 1H), 3.07-2.87 (m, 2H), 2.81-2.69 (m, 2H), 2.67-2.37 (m, 3H), 2.22-2.02 (m, 2H), 2.00-1.81 (m, 4H), 1.74-1.53 (m, 4H), 1.30 (t, J=7.6 Hz, 3H), 1.17 (d, J=6.8 Hz, 3H). ¹⁹F NMR (377 MHz, METHANOL-d4) δ=−115.41 (br d, J=42.6 Hz, 2F)

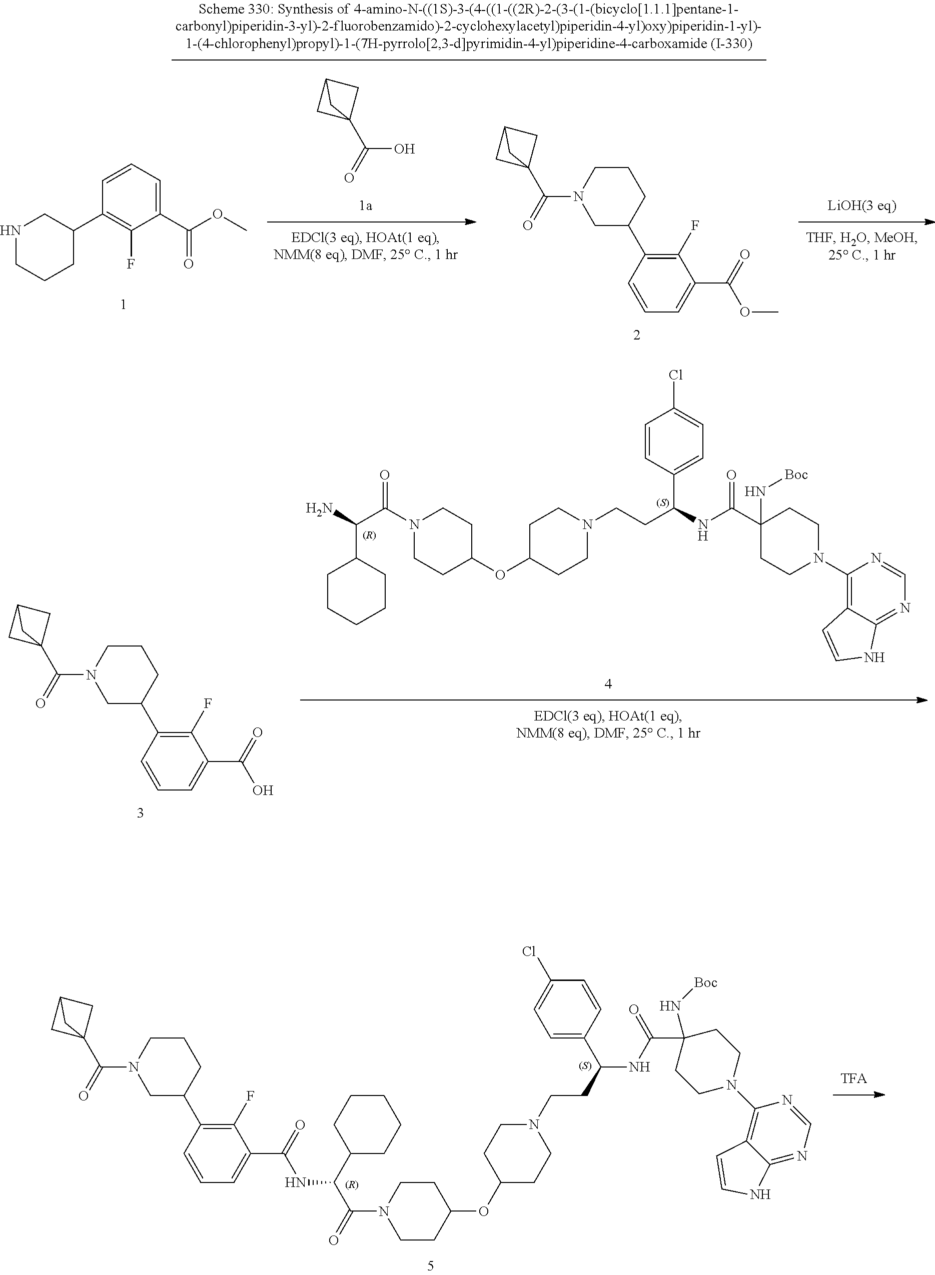

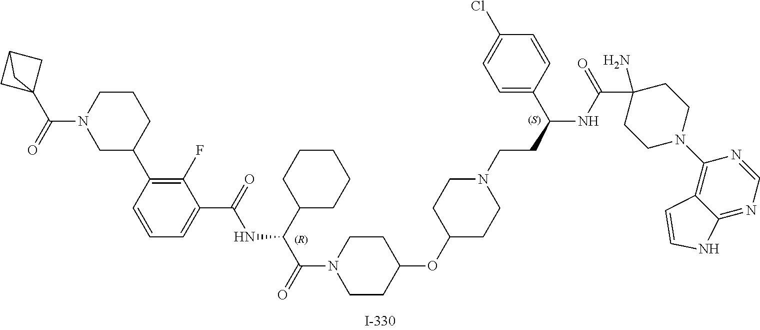

### Step 1: Synthesis of 2

To a solution of 1 (500 mg, 2.11 mmol, 1 eq) in DMF (5 mL) was added EDCI (1.21 g, 6.32 mmol, 3 eq), NMM (1.07 g, 10.54 mmol, 1.16 mL, 5 eq), 1a (259.91 mg, 2.32 mmol, 1.1 eq) and HOAt (286.83 mg, 2.11 mmol, 294.79 μL, 1 eq). The mixture was stirred at 25° C. for 0.5 hr. (SiO₂, by UV 254 nm, PE:EA=0:1, Rf=0.8). The reaction mixture was diluted with water (10 mL) at 25° C., and mixture was extracted with EA (10 mL*3) and combined organic phase was dried with anhydrous sodium sulfate, filtered and filtrate was concentrated to give crude product. The crude product was purified by column chromatography (SiO₂, PE/EA=1/0 to 0/1) and the eluent was concentrated under reduced pressure to give product. 2 (600 mg, 1.77 mmol, 84.20% yield, 98% purity) was obtained as a colorless oil. LCMS: Rt=0.473 min, [M+H]⁺=332.5.

### Step 2: Synthesis of 3

To a solution of 2 (580 mg, 1.75 mmol, 1 eq) in MeOH (2 mL), THF (2 mL) and H₂O (2 mL) was added LiOH·H₂O (220.34 mg, 5.25 mmol, 3 eq). The mixture was stirred at 25° C. for 0.5 hr. The mixture was added water (5 mL) and adjusted to pH=5˜6 with aq. HCl (1 mol/L), and the mixture was extracted with EA (5 ml*3), and combined organic phase was dried with anhydrous sodium sulfate, filtered and filtrate was concentrated to give product (450 mg, crude). No need further purification and the crude product was used in the next step directly. LCMS: Rt=0.380 min, [M+H]⁺=318.1.

### Step 3: Synthesis of 5

To a solution of 3 (140 mg, 441.15 μmol, 1 eq) in DMF (4.0 mL) was added EDCI (253.71 mg, 1.32 mmol, 3.0 eq) and HOAt (90.07 mg, 661.72 μmol, 92.57 μL, 1.5 eq) and NMM (223.10 mg, 2.21 mmol, 242.50 μL, 5.0 eq). Then 4 (368 mg, 441.15 μmol, 1 eq) was added, the mixture was stirred at 25° C. for 1 hr. The combined mixture was diluted with water (10 mL) and extracted with EA (10 mL*3). The organic layer was washed with brine (20 mL) and concentrated under vacuum. The crude product was purified by reverse phase (120 g of XB—C18, 20-40 μm, 120 Å) Mobile phase: A for H₂O (0.1% FA v/v) and B for acetonitrile; Gradient: B 5%-40% in 15 min; Flow rate: 80 ml/min; Column temperature: R.T. Wavelength: 220 nm/254 nm) and dried by lyophilization. 5 (420 mg, 368.77 μmol, 83.59% yield, 99.55% purity) was obtained as white solid. LCMS: Rt=0.476 min, [1/2M+H]=567.8. SFC: peak 1/peak=1/1, Method details: “Column: (S. S) Whelk-O1 50*4.6 mm I.D., 3.5 um, Mobile phase: Phase A for CO₂, and Phase B for MeOH+MeCN (0.05% DEA); Gradient elution: 60% MeOH+Me CN (0.05% DEA) in CO₂, Flow rate: 3 mL/min: Detector: PDA; Column Temp: 35° C.: Back Pressure: 100 Bar”

Step 4: Synthesis of I-330 To a solution of 5 (350 mg, 308.69 μmol, 1 eq) in DCM (6.0 mL) was added TFA (3.07 g, 26.92 mmol, 87.22 eq). The mixture was stirred at 25° C. for 0.5 hr. The combined reaction mixture was diluted with EA (30 mL) and washed with brine (10 mL). The organic layer was collected and concentrated under vacuum to give crude product. The crude product was purified by reverse phase (120 g of XB—C18, 20-40 μm, 120 Å) Mobile phase: A for H₂O (0.1% FA v/v) and B for acetonitrile; Gradient: B 5%-30% in 15 min: Flow rate: 80 ml/min; Column temperature: R.T. Wavelength: 220 nm/254 nm) and dried by lyophilization. I-330 (178.24 mg, 164.12 μmol, 53.17% yield, 99.42% purity, FA) was obtained as white solid. LCMS: Rt=0.460 min, [1/2M+H]⁺=517.8. Special LCMS: Rt₁=13.368 mins, peak 1:54.41%, Rt₂=13.561 mins, peak 2:45.01%. SFC: Method details: “Column: (S, S) Whelk-O1 50*4.6 mm I.D., 3.5 um, Mobile phase: Phase A for CO₂, and Phase B for EtOH+ACN (0.05% DEA); Gradient elution: 60% EtOH+ACN (0.05% DEA) in CO₂, Flow rate: 3 mL/min; Detector: PDA; Column Temp: 35 C; Back Pressure: 100 Bar”, peak 1:43.625%, peak 2:56.375%, ¹H NMR (400 MHZ, DMSO-d₆) δ=11.66 (s, 1H), 8.66 (s, 1H), 8.55-8.29 (m, 1H), 8.16 (s, 1H), 8.12 (s, 1H), 7.54-7.19 (m, 7H), 7.18-7.12 (m, 1H), 6.58 (br s, 1H), 4.89-4.70 (m, 2H), 4.48-4.31 (m, 3H), 4.28-4.11 (m, 1H), 4.06-3.78 (m, 3H), 3.70-3.56 (m, 3H), 3.18-2.80 (m, 5H), 2.75-2.58 (m, 3H), 2.48-2.38 (m, 2H), 2.20 (s, 2H), 2.15-1.96 (m, 9H), 1.92-1.64 (m, 14H), 1.61 (s, 2H), 1.54-1.34 (m, 6H), 1.32-1.22 (m, 1H), 1.20-0.93 (m, 5H), ¹⁹F NMR (377 MHz, DMSO-d₆) δ=−73.41 (s, 1F), −119.44-−120.61 (m, 1F),

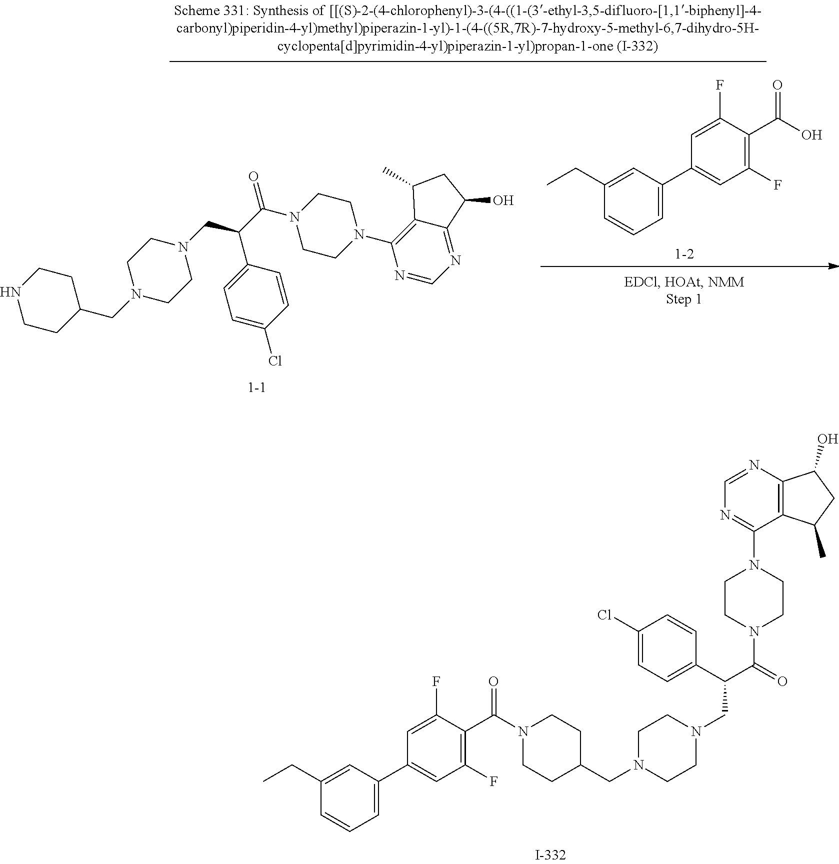

### Step 1: Synthesis of I-332

A mixture of 1-1 (320 mg, 517.26 μmol, 1 eq, HCl salt), 1-2 (320.00 mg, 1.22 mmol, 2.36 eq), EDCI (298 mg, 1.55 mmol, 3.01 eq), HOAt (106 mg, 778.78 μmol, 1.51 eq) and NMM (419 mg, 4.14 mmol, 455.43 μL, 8.01 eq) in DMF (4 mL) was degassed and purged with N₂ for 3 times, and then the mixture was stirred at 25° C. for 1 hr under N₂ atmosphere. The reaction mixture was diluted with water (50 mL) and extracted with EtOAc (25 mL*2). The combined organic layers were washed with brine (50 mL*2), dried over Na₂SO₄, filtered and concentrated under reduced pressure to give a residue. The crude product was purified by reversed-phase HPLC (0.1% NH₃·H₂O) and SFC separation (column: DAICEL CHIRALPAK AD (250 mm*30 mm, 10 um); mobile phase: [CO₂-ACN/i-PrOH (0.1% NH₃H₂O)]; B %: 50%, isocratic elution mode) to give I-332 (191.31 mg, 231.49 μmol, 83.18% yield, 100% purity) as a white solid. LCMS: Rt=1.153 min, [M+H]⁺=826.5. HPLC: Rt=11.453 min. ¹H NMR (400 MHz, DMSO-d₆) δ=8.43 (s, 1H), 7.63-7.52 (m, 4H), 7.43-7.33 (m, 5H), 7.29 (d, J=7.6 Hz, 1H), 5.38 (d, J=5.5 Hz, 1H), 4.83 (m, 1H), 4.48 (br d, J=12.8 Hz, 1H), 4.30 (br t, J=6.8 Hz, 1H), 3.73 (m, 1H), 3.69-3.55 (m, 3H), 3.54-3.36 (m, 7H), 3.31-3.26 (m, 1H), 3.09 (br t, J=11.6 Hz, 1H), 3.00 (m, 1H), 2.87-2.77 (m, 1H), 2.68 (m, 2H), 2.42 (m, 4H), 2.27 (m, 3H), 2.14-2.03 (m, 2H), 2.01-1.87 (m, 2H), 1.85-1.73 (m, 2H), 1.72-1.63 (m, 1H), 1.23 (t, J=7.6 Hz, 3H), 1.04 (d, J=6.8 Hz, 3H), 1.02-0.90 (m, 2H). ¹⁹F NMR (377 MHz, DMSO-d₆) δ=−114.04 (br d, J=4.5 Hz, 2F). SFC: Rt=0.773 min, de value=99%

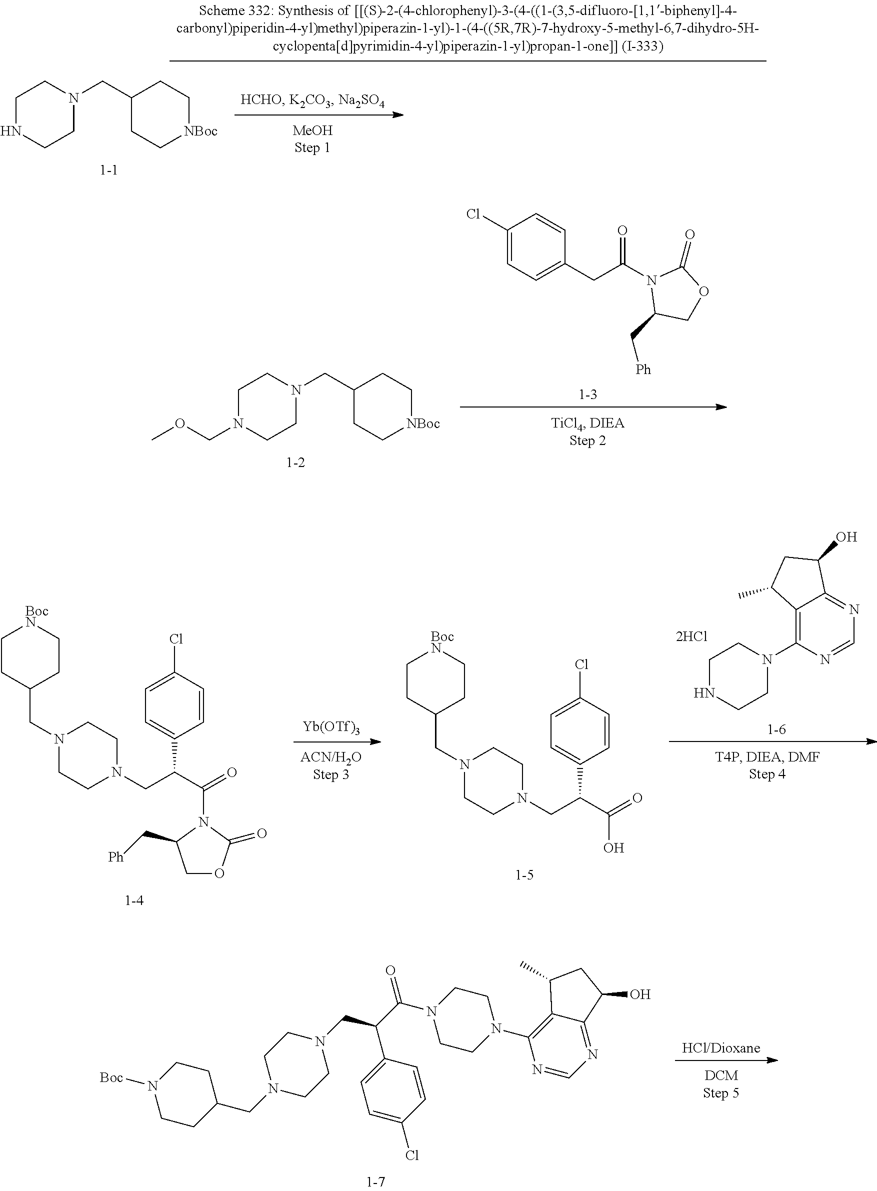

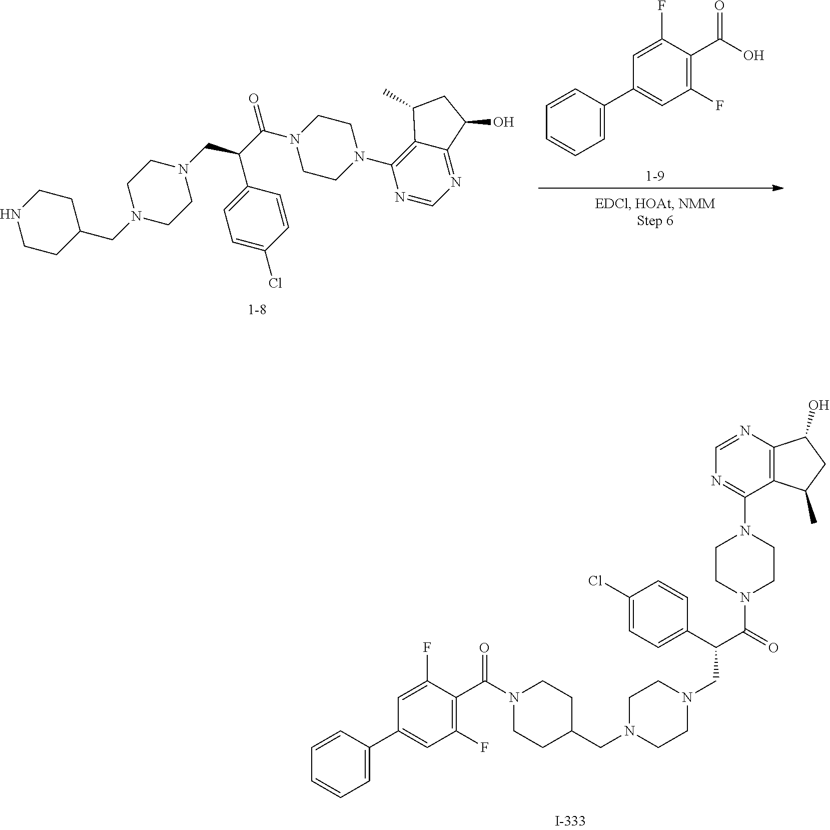 [SMILES: C[C@@H]1C[C@@H](O)c2ncnc(N3CCN(C(=O)[C@H](CN4CCN(CC5CCNCC5)CC4)c4ccc(Cl)cc4)CC3)c21 ; O=C(O)c1c(F)cc(-c2ccccc2)cc1F ; "1-9" ; C[C@@H]1C[C@@H](O)c2ncnc(N3CCN(C(=O)[C@H](CN4CCN(CC5CCN(C(=O)c6c(F)cc(-c7ccccc7)cc6F)CC5)CC4)c4ccc(Cl)cc4)CC3)c21]

### Step 1: Synthesis of 1-2

To a solution of 1-1 (5 g, 17.64 mmol, 1 eq) and formaldehyde (583.00 mg, 19.42 mmol, 534.86 μL, 1.1 cg) in MeOH (50 mL) was added Na₂SO₄ (5.01 g, 35.28 mmol, 3.58 mL, 2 eq) and K₂CO₃ (4.88 g, 35.28 mmol, 2 eq). Then the reaction mixture was stirred at 25° C. for 16 hr. The reaction mixture was filtered and concentrated to remove the solvent. Then the mixture was added DCM (50 mL) and stirred for 30 min. filtered and concentrated to afford 1-2 (5 g. crude) as a yellow solid which was used into the next step without further purification. ¹H NMR (400 MHZ, METHANOL-d4) δ=4.05 (br d, J=13.2 Hz, 2H). 4.00 (s, 2H), 3.35 (s, 1H), 3.32 (s, 2H), 2.71 (t. J=4.8 Hz. 5H), 2.59-2.36 (m, 4H), 2.20 (d, J=6.6 Hz. 2H), 1.80-1.68 (m, 3H), 1.45 (s. 9H), 1.13-0.96 (m, 2H).

### Step 2: Synthesis of 1-4

To a solution of 1-3 (5 g. 15.16 mmol, 1 eq) in DCM (160 mL) was added TiCl, (3.02 g. 15.92 mmol, 1.75 mL, 1.05 cg) and DIEA (2.16 g. 16.68 mmol, 2.90 mL, 1.1 cg) at −70° C. under N₂. The mixture was stirred at −70° C. for 0.5 hr. The reaction mixture color was turned to dark blue. Then 1-2 (4.96 g. 15.16 mmol, 1 cg) in DCM (40 mL) was added into the mixture. The reaction mixture was stirred at 25° C. for 2 hrs. Then the reaction mixture color was turned to yellow. The reaction mixture was poured into saturated solution of NH₄Cl (100 mL), a lot of yellow precipitates formed and the mixture color turned to yellow, the suspension was filtered, the filter liquid was extracted with DCM (3*100 mL). The combined organic phase was dried by Na₂SO₄, filtered and concentrated to give the residue. The residue was purified by flash silica gel chromatography (ISCO®; 120 g SepaFlash® Silica Flash Column, Eluent of 30˜60% Ethylacetate/(Petroleum/dichloromethane=3/1, contained 1% NH₃·H₂O) ether gradient (@, 100 mL/min) to give 1-4 (4.0 g. 5.95 mmol, 39.24% yield, 93% purity) as a white solid. LCMS: Rt=2.686 min. [M+H]⁺=625.4 LCMS: Rt=2.687 min, [M+H]⁺=625.4 HPLC: Rt=4.622 min ¹H NMR (400 MHZ, CHLOROFORM-d) δ=7.36 (m, 4H), 7.33-7.28 (m, 5H), 5.50 (dd, J=4.4, 11.0 Hz, 1H), 4.70-4.62 (m, 1H), 4.16-4.05 (m, 4H), 3.44-3.25 (m, 2H), 2.91-2.81 (m, 1H), 2.77-2.61 (m, 4H), 2.59-2.46 (m, 3H), 2.40 (br s, 4H), 2.14 (br d, J=5.2 Hz, 2H), 1.75-1.62 (m, 3H), 1.45 (s. 9H), 1.13-1.00 (m, 2H). SFC: Rt=0.837 min, de value=99.3% Step 3: Synthesis of 1-5

To a solution of 1-4 (2.6 g. 4.16 mmol, 1 eq) in MeCN (26 mL) and water (7 mL) was added tris(trifluoromethylsulfonyloxy) ytterbium (260.00 mg, 419.19 μmol, 0.10 eq). The reaction mixture was stirred at 105° C. for 3 hr. The reaction was concentrated to afford crude product. The crude product was purified by reversed-phase HPLC (0.1% FA condition), the eluent was lyophilized to give 1-5 (650 mg. 1.35 mmol, 32.42% yield. 96.66% purity) as a yellow solid. LCMS: Rt=0.574 min. [M+H]⁺=466.51H NMR (400 MHZ, CHLOROFORM-d) δ=7.32 (d, J=8.4 Hz, 2H), 7.22-7.16 (m, 2H), 4.14-3.99 (m, 2H), 3.80 (dd, J=4.2, 12.0 Hz, 1H), 3.33-3.19 (m, 1H), 3.14-2.92 (m, 2H), 2.92-2.78 (m, 2H), 2.77-2.52 (m, 7H), 2.31-2.19 (m, 2H), 1.78-1.58 (m, 3H), 1.46 (s. 9H), 1.14-0.99 (m, 2H). SFC: Rt=1.586 min. ee value=98.5%

### Step 4: Synthesis of 1-7

To a solution of 1-6 (396.00 mg, 1.29 mmol, 1.5 eq. 2HCl salt), 1-5 (400 mg, 858.35 μmol, 1 eg) in DMF (8 mL) was added TIP (1.86 g. 2.58 mmol, 50% purity in EA, CAS: 163755-62-2, 3 eq) and DIEA (560.00 mg, 4.33 mmol, 754.72 μL, 5.05 eq) at 25° C. The mixture was stirred at 25° C. for 1 hr. The reaction mixture was diluted with water (50 mL) and extracted with EtOAc (25 mL*2). The combined organic layers were washed with brine (50 mL*2), dried over Na₂SO₄, filtered and concentrated under reduced pressure to give a residue. The residue was purified by reversed-phase HPLC (0.1% FA condition), the eluent was lyophilized to give 1-7 (440 mg, 624.63 μmol, 72.77% yield, 96.86% purity) as a yellow solid. LCMS: Rt=0.446 min, [M+H]⁺=682.3. HPLC: Rt=0.871 min. ¹H NMR (400 MHZ, CHLOROFORM-d) δ=8.53 (s, 1H), 7.34-7.30 (m, 2H), 7.27-7.23 (m, 2H), 5.11 (t. J=7.1 Hz, 1H). 4.13-3.97 (m, 3H), 3.91-3.78 (m, 2H), 3.75-3.62 (m, 2H), 3.57 (br s, 2H), 3.54-3.43 (m, 2H), 3.33 (td, J=8.3, 12.6 Hz, 2H), 2.76-2.62 (m, 2H), 2.60-2.46 (m, 5H), 2.39 (br d, J=3.4 Hz, 4H), 2.21-2.12 (m, 4H), 1.72 (br s, 3H), 1.47 (s. 9H), 1.18 (d, J=6.9 Hz, 3H), 1.12-1.00 (m, 2H). SFC: Rt=1.850 min. de value=95% Step 5: Synthesis of 1-8

To a solution of 1-7 (400 mg, 586.26 μmol, 1 eg) in DCM (4 mL) was added HCl/dioxane (4 mL, 2M). The mixture was stirred at 25° C. for 1 hr. The reaction was concentrated to afford 1-8 (360 mg, crude. HCl salt) as a white solid which was used into the next step without further purification. LCMS: Rt=0.993 min. [M+H]⁺=582.3 Step 6: Synthesis of 1-333

A mixture of 1-8 (360 mg, 581.92 μmol, 1 eq. HCl salt), 1-9 (164 mg, 700.26 μmol, 1.2 eq). EDCI (340.00 mg, 1.77 mmol, 3.05 eq). HOAt (80.00 mg, 587.76 μmol, 82.22μ. 1.01 eq) in DMF (5 mL) was added NMM (480.00 mg, 4.75 mmol, 521.74 μL, 8.15 eq) dropwise, then the mixture was degassed and purged with N₂ for 3 times, and then the mixture was stirred at 25° C. for 1 hr under No atmosphere. The reaction mixture was diluted with water (50 mL) and extracted with EtOAc (25 mL*2). The combined organic layers were washed with brine (50 mL*2), dried over Na₂SO₄, filtered and concentrated under reduced pressure to give a residue. The residue was purified by reversed-phase HPLC (0.1% NH₂·H₂O), the eluent was lyophilized to give I-333 (214.88 mg, 269.15 μmol, 46.25% yield. 100% purity) as a white solid. LCMS: Rt=0.467 min, [M+H]⁺=798.3. HPLC: Rt=10.222 min. ¹H NMR (400 MHZ, DMSO-d₆) δ=8.43 (s, 1H), 7.81-7.74 (m, 2H), 7.60-7.54 (m, 2H), 7.52-7.42 (m, 3H), 7.36 (s. 4H), 5.38 (d, J=5.6 Hz. 1H), 4.83 (q. J=6.4 Hz. 1H), 4.48 (d, J=12.8 Hz, 1H), 4.30 (t. J=6.8 Hz, 1H), 3.77-3.70 (m, 1H). 3.68-3.55 (m, 3H), 3.49-3.40 (m, 7H), 3.32 (s, 1H), 3.13-3.05 (m, 1H), 3.04-2.97 (m, 1H), 2.88-2.77 (m, 1H), 2.46-2.38 (m, 4H), 2.27 (br s, 3H), 2.14-2.03 (m, 2H), 2.01-1.87 (m, 2H), 1.85-1.72 (m, 2H), 1.72-1.62 (m, 1H), 1.04 (d, J=6.8 Hz, 3H), 1.02-0.90 (m, 2H). ¹⁹F NMR (376 MHz, DMSO-d₆) δ=−113.91 (brs, 2F). SFC: Rt=2.335 min, de value=95.1% 2

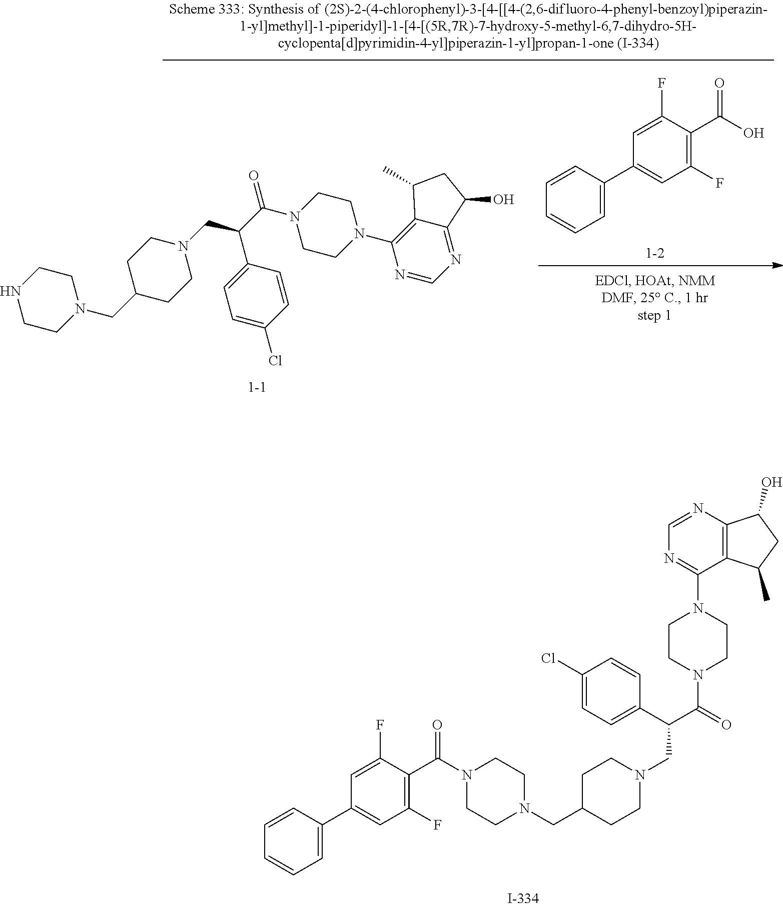 [SMILES: C[C@@H]1C[C@@H](O)c2ncnc(N3CCN(C(=O)[C@H](CN4CCC(CN5CCNCC5)CC4)c4ccc(Cl)cc4)CC3)c21 ; O=C(O)c1c(F)cc(-c2ccccc2)cc1F ; "1-2" ; C[C@@H]1C[C@@H](O)c2ncnc(N3CCN(C(=O)[C@H](CN4CCC(CN5CCN(C(=O)c6c(F)cc(-c7ccccc7)cc6F)CC5)CC4)c4ccc(Cl)cc4)CC3)c21]

### Step 1: Synthesis of I-334

A mixture of 1-1 (300 mg, 484.94 μmol, 1 eq, HCl salt) and 1-2 (113.57 mg, 484.94 μmol, 1 eq) in DMF (3 mL) were added EDCI (278.89 mg, 1.45 mmol, 3 eq) and HOAt (99.01 mg, 727.40 μmol, 101.75 μL, 1.5 eq). Then NMM (245.26 mg, 2.42 mmol, 266.58 μL, 5 eq) was added. The reaction solution was stirred at 25° C. for 1 hr. The reaction mixture was diluted with water 5 mL and extracted with EA (5 mL*3). The combined organic layers were washed with brine (5 mL*3), dried over [Na₂SO₄], filtered and concentrated under reduced pressure to give a residue. The residue was purified by reversed phase (ISCO®; 40 g SepaFlash® C18 Column, Eluent of 5˜50%˜95% (0.1% FA) water/MeCN (@ 40 mL/min), the major peak was collected and combined with another batch. The two batches were diluted with sat. aq. NaHCO₃ (10 mL) and extracted with EA (50*3 mL). The combined organic layers were dried over [Na₂SO₄], filtered and concentrated under reduced pressure to give a residue. The residue was repurified by reversed phase (ISCO®; 40 g SepaFlash® C18 Column, Eluent of 5˜70%˜95% (0.1% NH₃·H₂O) water/MeCN @ 40 mL/min), the eluent was collected and concentrated to remove MeCN, then lyophilized to I-334 (185.71 mg, 230.33 μmol, 47.50% yield, 99.02% purity) as a white solid. LCMS: Rt=0.452 min, [M+H]⁺=798.4. HPLC: Rt=7.765 min. SFC: Rt=1.904 min, de value=93.9%. ¹H NMR (400 MHZ, DMSO-d₆) δ=8.43 (s, 1H), 7.83-7.72 (m, 2H), 7.58 (d, J=8.8 Hz, 2H), 7.54-7.42 (m, 3H), 7.36 (s, 4H), 5.38 (d, J=5.6 Hz, 1H), 4.86-4.81 (m, 1H), 4.30 (t, J=6.8 Hz, 1H), 3.79-3.69 (m, 1H), 3.68-3.54 (m, 4H), 3.53-3.36 (m, 4H), 3.27 (d, J=4.0 Hz, 3H), 3.04-2.99 (m, 1H), 2.86 (d, J=1.2 Hz, 2H), 2.44-2.33 (m, 3H), 2.28 (br s, 2H), 2.10 (d, J=7.2 Hz, 2H), 2.03-1.83 (m, 4H), 1.61 (s, 2H), 1.43-1.41 (m, 1H), 1.05 (d, J=6.8 Hz, 5H). ¹⁹F NMR (377 MHz, DMSO-d₆) δ=−113.69 (s, 1F).

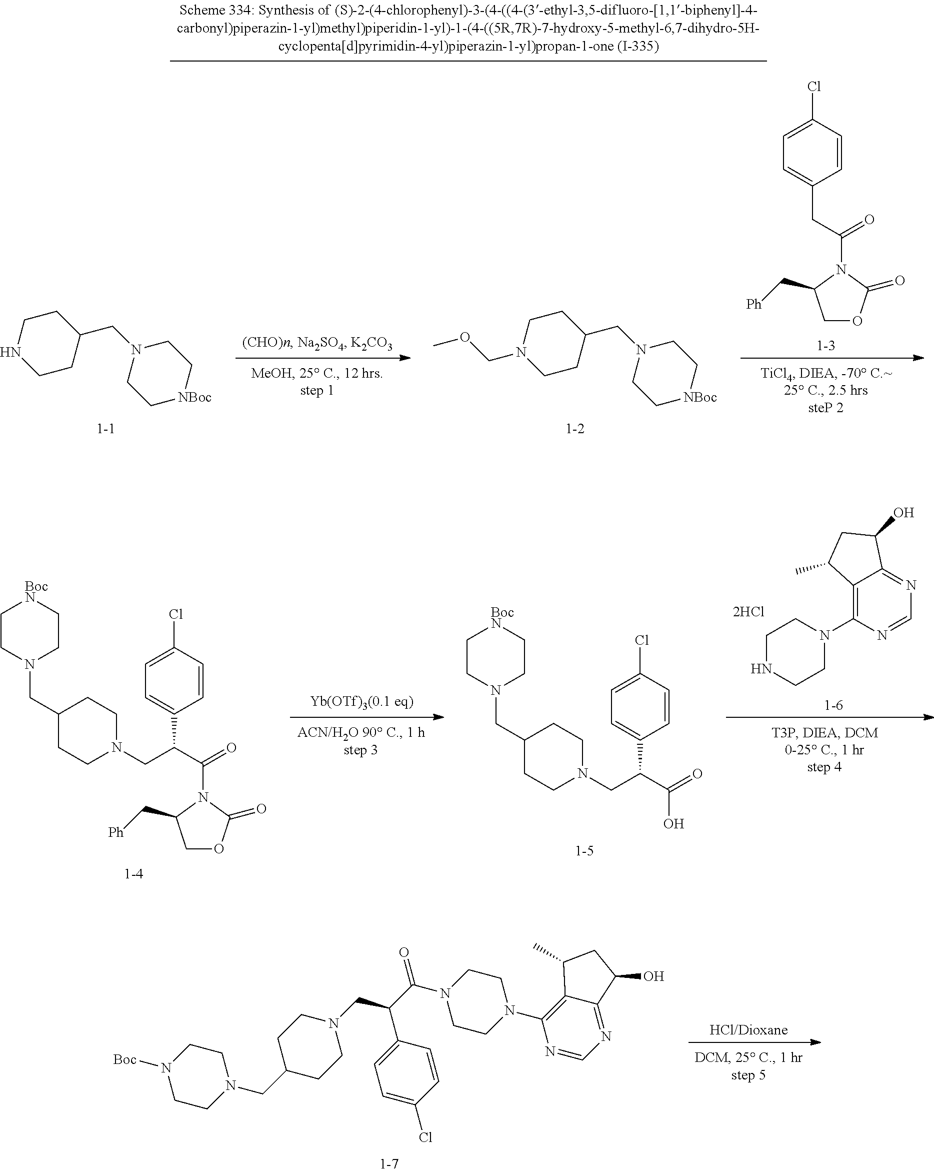

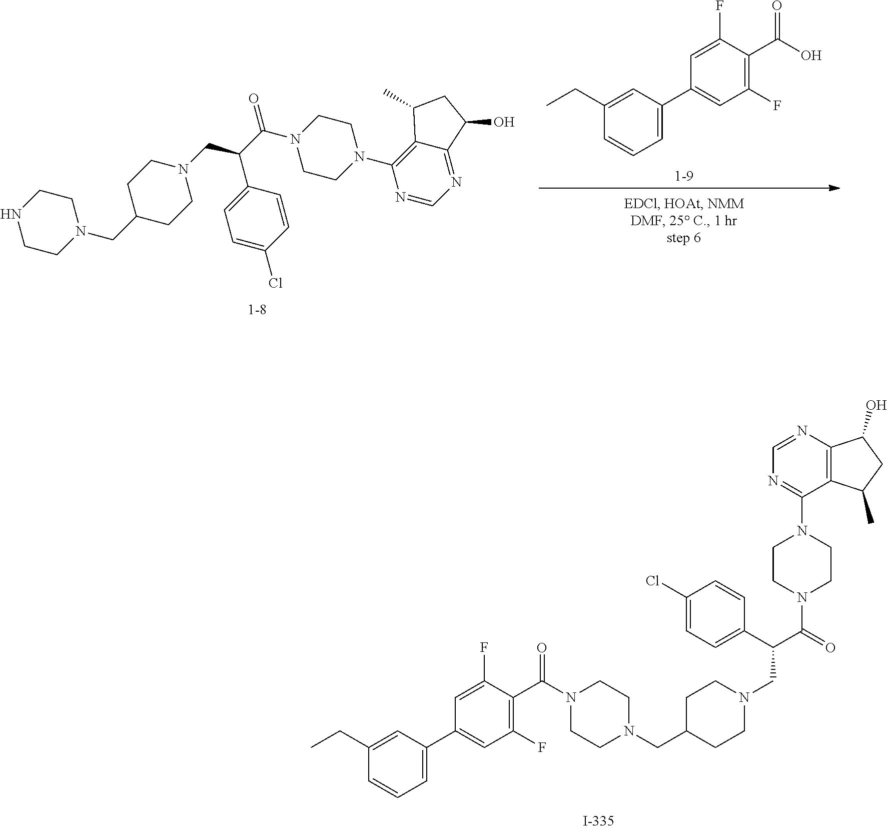

### Step 1: Synthesis of 1-2

To a solution of 1-1 (10 g. 35.28 mmol, 1 eq) and HCHO (1.06 g. 35.28 mmol, 971.98 μL, 1 eq) in MeOH (100 mL) was added Na₂SO₄ (10.02 g. 70.57 mmol, 7.16 mL, 2 eq) and K₂CO₃ (9.75 g. 70.57 mmol, 2 eq). The reaction mixture was stirred at 25° C. for 12 hrs. The reaction mixture concentrated to give the residue, the residue was diluted with DCM (100 mL), the suspension was filtered, the filter cake was washed with DCM (2*50 mL), the filter liquid was collected and concentrated to give 1-2 (11.5 g. 35.12 mmol, 99.53% yield) as colorless oil which was used into next step directly without any other purification. ¹H NMR (400 MHZ, METHANOL-d4) δ=4.01 (s, 2H), 3.41 (br s, 4H), 3.35 (s, 3H), 2.93-2.91 (m, 2H), 2.54-2.32 (m, 6H), 2.20 (d, J=7.2 Hz, 2H), 1.80-1.76 (m, 2H), 1.60-1.51 (m, 1H), 1.45 (s, 9H), 1.27-1.12 (m, 2H).

### Step 2: Synthesis of 1-4

To a solution of 1-3 (11.58 g, 35.12 mmol, 1 eq) in DCM (350 mL) was added TiCl₄ (6.99 g. 36.87 mmol, 4.05 mL, 1.05 eq) and TEA (4.99 g. 38.63 mmol, 6.73 mL, 1.1 eq) at −70° C. under N₂. The mixture was stirred at −70° C. for 0.5 hr. The reaction mixture color was turned to dark blue. Then 1-2 (11.5 g. 35.12 mmol, 1 eq) was added into the mixture. The reaction mixture was stirred at 25° C. for 1 hr. The reaction mixture was quenched with saturated solution of NH₄Cl (100 mL), a lot of white precipitates formed and the mixture color turned to yellow, the suspension was filtered, the filter liquid was extracted with DCM/MeOH (5/1, 5*100 mL). The combined organic phase was dried by Na₂SO₄, filtered and concentrated to give the residue. The residue was purified by flash silica gel chromatography (ISCO®; 330 g SepaFlash® Silica Flash Column, Eluent of 0˜40% DCM/(DCM/MeOH)=1/1, contained 1% NH₃·H₂O) ether gradient @ 100 mL/min) to give the two spots, but it contained some impurities. The spot 1 was re-purified by flash silica gel chromatography (ISCO®; 120 g SepaFlash® Silica Flash Column, Eluent of 40˜80% (DCM/PE=3/1, contained 1% NH₃·H₂O)/EA ether gradient @ 100 mL/min) to give the product 1 (1-4). The spot 2 was purified by flash silica gel chromatography (ISCO®; 120 g SepaFlash® Silica Flash Column, Eluent of 0˜20% DCM/(DCM/MeOH=1/1, contained 1% NH₃·H₂O) ether gradient @ 100 mL/min) to give the product 2 (4.6 g. 5.29 mmol, 15.06% yield. 71.9% purity), 1-4 (5.29 g. 7.52 mmol, 21.42% yield, 88.9% purity) was obtained as a white solid. LCMS: Rt=2.586 min, [M+H]⁺=625.5. ¹H NMR (400 MHZ, CHLOROFORM-d) ¿=7.39-7.27 (m, 11H), 7.21-7.17 (m, 2H), 5.52-5.48 (m, 1H), 4.92-4.91 (m, 1H), 4.72-4.60 (m, 1H), 4.50 (t. J=8.4 Hz, 1H), 4.23-4.05 (m, 4H), 3.48-3.27 (m, 5H), 3.16-3.12 (m, 1H), 2.99-2.79 (m, 4H), 2.53-2.48 (m, 1H), 2.31 (br s, 4H), 2.23-2.08 (m, 3H), 2.03-1.92 (m, 1H), 1.81-1.66 (m, 2H), 1.46 (s, 9H), 1.23-1.07 (m, 2H). ¹H NMR (400 MHZ, CHLOROFORM-d) δ=7.41-7.35 (m, 2H), 7.35-7.28 (m, 3H), 7.24-7.17 (m, 3H), 7.01-6.94 (m, 2H), 5.42-4.38 (m, 1H), 4.84-4.70 (m, 1H), 4.27-4.19 (m, 1H), 4.14-4.12 (m, 1H), 4.12-4.10 (m, 0.5H), 3.89-3.79 (m, 0.5H), 3.68 (s, 1.5H), 3.46-3.29 (m, 7H), 3.18-2.99 (m, 2.5H), 2.98-2.80 (m, 2H), 2.67-2.60 (m, 1H), 2.58-2.41 (m, 2H), 2.31 (brs, 6H), 2.20-2.06 (m, 5H), 2.02-1.88 (m, 2H), 1.75-1.57 (m, 10H), 1.22-1.05 (m, 3H)

### Step 3: Synthesis of 1-5

To a solution of 1-4 (5.29 g, 7.52 mmol, 1 eq) in mixture of MeCN (40 mL) and H₂O (20 mL) was added Yb(oTf)₃ (466.56 mg, 752.21 μmol, 0.1 eq). The reaction mixture was stirred at 90° C. for 1 hr under N₂ atmosphere. The reaction mixture was concentrated to give the residue. The residue was purified by reverse-phase (330 g of SepaFlash® Spherical C18, 20-45 μm, 100 Å; mobile phase: A for H₂O+0.1% FA and B for acetonitrile; gradient: B 5-15% in 45 min, flow rate: 200 mL/min; column temperature: RT, wavelength: 220 nm/254 nm), the eluent was concentrated to remove organic solvents, and lyophilized to get 1-5 (1.5 g. 2.93 mmol, 38.94% yield) as a white solid. LCMS: Rt=0.998 min, [M+H]⁺=466.4. SFC: Rt=1.113 min, 1.272 min, 94.3% ee value. ¹H NMR (400 MHZ, DMSO-d₆) δ=8.17 (s, 1H), 7.39-7.27 (m, 4H), 3.85-3.81 (m, 1H), 3.28 (br s, 4H), 3.20-3.15 (m, 1H), 3.10-3.04 (m, 1H), 3.00-2.96 (m, 1H), 2.63-2.58 (m, 1H), 2.34-2.08 (m, 8H), 1.73-1.69 (m, 2H), 1.61-1.48 (m, 1H), 1.38 (s, 9H), 1.18-1.01 (m, 2H)

### Step 4: Synthesis of 1-7

To a solution of 1-5 (500 mg, 1.02 mmol, 1 eq, 0.5 FA salt) and 1-6 (628.23 mg, 2.04 mmol, 2 eq, 2HCl salt) in DMF (5 mL) was added T₄P (1.47 g. 2.04 mmol, 50% purity in EA, 2 eq), followed by DIEA (660.72 mg, 5.11 mmol, 890.45 μL, 5 eq) was added at 0° C. The mixture was stirred at 25° C. for 1 hr. The reaction mixture was quenched by addition MeOH (0.5 mL) at 0° C., and then diluted with H₂O (10 mL) and extracted with DCM (10 mL*3). The combined organic layers were dried over [Na₂SO₄], filtered and concentrated under reduced pressure to give a residue. The residue was purified by reversed phase (ISCO®; 120 g SepaFlash® C18 Column, Eluent of 5˜20%˜95% (0.1% FA) water/MeCN @ 40 mL/min), the major peak was collected and lyophilized to get 1-7 (700 mg, 789.98 μmol, 77.26% yield, 77% purity) as a white solid. LCMS: Rt=0.392 min, [M+H]⁺=682.3. SFC: Rt=1.699 min, de value 85%, ¹H NMR (400 MHz, CHLOROFORM-d) δ=8.46 (s, 1H), 8.44 (br s, 1H), 7.35-7.27 (m, 4H), 5.16-5.06 (m, 2H), 4.95-4.63 (m, 4H), 3.94-3.79 (m, 2H), 3.78-3.63 (m, 3H), 3.57-3.26 (m, 9H), 2.94 (d, J=11.4 Hz, 1H), 2.72-2.48 (m, 2H), 2.42-2.27 (m, 4H), 2.26-2.08 (m, 4H), 1.89 (t, J=12.4 Hz, 2H), 1.73-1.49 (m, 3H), 1.46 (s, 9H), 1.12 (d, J=7.2 Hz, 2H).

### Step 5: Synthesis of 1-8

A solution of 1-7 (600 mg, 677.13 μmol, 1 eq) in DCM (6 mL) was added HCl/dioxane (4 M, 4.62 mL, 27.29 eq). The reaction solution was stirred at 25° C. for 1 hr. The reaction mixture was concentrated to get the residue. The residue was purified by reversed phase (ISCO®; 120 g SepaFlash R C18 Column, Eluent of 5˜20%˜95% (0.1% aq. HCl) water/MeCN @ 100 mL/min), the major peak was collected and lyophilized to get 1-8 (350 mg, 565.76 μmol, 83.55% yield, 100% purity; HCl salt) as a white solid. LCMS: Rt=0.359 min, [M+H]′=582.3. SFC: Rt=2.085 min. ¹H NMR (400 MHZ, DMSO-d₆) δ=12.16-11.30 (m, 1H), 10.98-10.48 (m, 1H), 10.20-9.46 (m, 2H), 8.75 (s, 1H), 7.52-7.46 (m, 2H), 7.45-7.38 (m, 2H), 5.28 (t, J=8.0 Hz, 1H), 4.98-4.79 (m, 1H), 4.13-4.04 (m, 2H), 3.94-3.84 (m, 6H), 3.42-3.19 (m, 7H), 3.15-3.02 (m, 3H), 3.00-2.85 (m, 2H), 2.20-2.08 (m, 3H), 2.07-1.99 (m, 2H), 1.83-1.63 (m, 2H), 1.51 (d, J=8.4 Hz, 1H), 1.48-1.38 (m, 3H), 1.38-1.27 (m, 2H), 1.13-1.06 (m, 3H)

### Step 6: Synthesis of I-335

A mixture of 1-8 (290 mg, 468.77 μmol, 1 eq, HCl salt) and 1-9 (135.23 mg, 515.65 μmol, 1.1 eq) in DMF (3 mL) were added EDCI (269.59 mg, 1.41 mmol, 3 eq) and HOAt (95.71 mg, 703.16 μmol, 98.36 μL, 1.5 eq). Then NMM (237.07 mg, 2.34 mmol, 257.69 μL, 5 eq) was added. The reaction solution was stirred at 25° C. for 1 hr. The reaction mixture was diluted with water (5 mL) and extracted with EA (5 mL*3). The combined organic layers were washed with brine (5 mL*3), dried over [Na₂SO₄], filtered and concentrated under reduced pressure to give a residue. The residue was purified by reversed phase (ISCO®; 40 g SepaFlash® C18 Column, Eluent of 5˜80%˜95% (0.1% NH₃·H₂O) water/MeCN @ 40 mL/min), the major peak was collected and lyophilized to get I-335 (152.09 mg, 178.96 μmol, 38.18% yield, 97.24% purity) as a white solid. LCMS: Rt=0.808 min, [M+H]′=826.6. HPLC: Rt=9.319 min. SFC: Rt=1.313 min, de value=93.5%, ¹H NMR (400 MHZ, DMSO-d₆) δ=8.43 (s, 1H), 7.64-7.52 (m, 4H), 7.44-7.33 (m, 5H), 7.29 (d, J=7.6 Hz, 1H), 5.38 (d, J=4.0 Hz, 1H), 4.97-4.73 (m, 1H), 4.30 (t, J=6.8 Hz, 1H), 3.78-3.69 (m, 1H), 3.68-3.58 (m, 4H), 3.54-3.44 (m, 3H), 3.29-3.22 (m, 4H), 3.03-2.97 (m, 1H), 2.86 (d, J=4.4 Hz, 2H), 2.69-2.67 (m, 2H), 2.45-2.32 (m, 4H), 2.28 (br s, 2H), 2.10 (br d, J=7.2 Hz, 2H), 2.02-1.84 (m, 4H), 1.68-1.56 (m, 2H), 1.51-1.35 (m, 1H), 1.23 (t, J=7.6 Hz, 3H), 1.11-0.94 (m, 5H). ¹⁹F NMR (377 MHz, DMSO-d₆) δ=−113.80 (s, 1F)

### Example 2: Biological Assays

### Biochemical IC₅₀ Determination and Kinase Profiling

Biochemical IC₅₀ determination for compounds against full-length AKT1, AKT2, and AKT3 was performed at Reaction Biology (Malvern, PA). Compounds were tested in a 10-dose mode with 3-fold serial dilutions starting at 1 μM in the presence of 10 μM ATP and the IC₅₀ was determined. Curve fits were performed where the enzyme activities at the highest concentration of compounds were less than 65% of the enzyme activity (relative to DMSO controls).

### Cell Culture Conditions

All cancer cell lines were grown in EMEM, DMEM or RPMI-1640 (Life Technologies Corporation, Carlsbad, CA) supplemented with 10% FBS. 293T and NIH 3T3 cells were cultured in DMEM with or without GlutaMAX, respectively, supplemented with 10% FBS. All cell cultures were maintained at 37° C. in a humidified atmosphere containing 5% CO₂.

### Expression DNA Constructs and Transfection

WT and E17K mutant forms of the human AKT1 cDNA were fused with green fluorescent protein (GFP) in the c-terminus and cloned into the pcDNA expression vector, generating pcDNAAKT1-WT-GFP and pcDNAAKT1-E17K-GFP.

Cells were transiently transfected with pcDNAAKT1-WT-GFP or pcDNAAKT1-E17K-GFP

### Western Blot Analyses

Cells (MDA-MB-453:1.5×10⁶: NCI-H1650: 1×10⁶: KU-19-19:0.7×10⁶) were plated into 6 well plates, left overnight, and then treated with full media containing different concentrations of Akt inhibitors. Cells were treated under designated conditions and lysates were extracted. Proteins were resolved from extracts using SDS-PAGE followed by immunoblotting. P-Akt (T308), p-Akt (S473), total Akt, p-PRAS40 (T246), total PRAS40, pGSK3b (S9), pAS160 (S318), pFOXO1 (T24)/3a (T32), pBAD (S136), pS6 (S235/S236), p4E-BP1 (S65), pERK (T202/Y204), and total ERK, were assessed using the corresponding primary antibodies.

Tumor tissues were ground in mortars with liquid nitrogen and lysed. Proteins extracted from tumor tissues were resolved using E-PAGE followed by immunoblotting. P-Akt (T308), p-Akt (S473), total Akt, p-PRAS40 (T246) and B-Actin were assessed with corresponding primary antibodies. Infrared dye conjugated secondary antibodies were used as secondary antibodies. The intensity of the bands was quantitated using the accompanying software. The data were expressed as percent inhibition in comparison to vehicle.

### Intrinsic Tryptophan Fluorescence Quench Assay

The binding of compounds to AKT1-WT and AKT1-E17K is followed by monitoring intrinsic tryptophan fluorescence quench. Inhibitors are serially diluted (two-fold) with buffer solution (25 mM Tris, pH 7.5, 0.1 M NaCl and 2% DMSO) in 384 well plates. AKT1 or AKT1-E17K (100 nM) is added to each well and diluted to a final volume of 50 μl with buffer. The resulting mixtures are allowed to equilibrate for 30 minutes at room temperature. The change in intrinsic emission is measured at an excitation wavelength of 280 nm and emission wavelength of 345 nm. The fluorescence at 345 nm is plotted versus the inhibitor concentration, and the data are fit to a quadratic equation using nonlinear regression. The following expression relating observed fluorescence (F) to inhibitor concentration [I₀] is used to obtain the binding constant for Akt inhibitors: (K_{D}^{app}), F=F₀+(ΔF/P₀)[(K_{D}^{app}+[P₀]+[I₀])−the square root of [(K_{D}^{app}+[P₀]+[I₀])²−4[P₀][I₀]]]/2, where P₀, is the initial protein concentration (constant), K_{D} is the dissociation constant, ΔF is the total change in fluorescence, and I₀ is the inhibitor concentration. Each titration experiment represents an average obtained from four independent experiments.

### AKT1 Plasma Membrane Translocation

Cells (15,000 cells per chamber) were plated in full media without antibiotics. Next, the cells were transfected with Akt-GFP plasmids using 1 μL Lipofectamine 2000 (0.81 μg of AKT1-E17K-GFP and 0.6 μg of AKT1-WT-GFP) in the presence of 30 μL Opti-MEM. After 48 hours, cells were incubated in serum free media for an additional 24 hours and then treated with or without inhibitor for 2 hours. Next, the cells were stimulated with PDGF at 50 ng/ml for 10 minutes. Cells were washed with PBS and then fixed with 200 μl paraformaldehyde fixation buffer (3% paraformaldehyde, 2% sucrose in PBS, pre-warmed at 60-70° C.). After washing with PBS, cells were blocked and permcabolized with 0.1% TX-100+1% BSA in PBS for 15 minutes at room temperature. Next, 200 μl DAPI (1 μg/ml in PBS) was used to counterstain the nuclei at room temperature in darkness for 15 minutes. Samples were then rinsed with cold PBS and stored at 4° C. until imaging. Images were captured using a microscope.

### OncoPanel Analysis

Anti-proliferative activity of compounds is assessed using a Panel such as 240 (Eurofins, St. Charles, MO). Cells are seeded into 384-well plates and incubated in a humidified atmosphere of 5% CO₂ at 37° C. Compounds are added 24 hours post cell seeding. At the same time, a time zero untreated cell plate is generated. Compounds are serially diluted and assayed over a range of concentrations in buffer containing 0.1% DMSO. After a 72 hour incubation period, cells are fixed and stained with fluorescently labeled antibodies and nuclear dye to determine relative cell number. Images are acquired and analyzed. GI₅₀ values are calculated.

### Mutation Analysis

Mutation analysis is performed based on information from the COSMIC database (http://cancer.sanger.ac.uk/cancergenome/projects/cosmic/). Cells with GI₅₀<1 μM are designated as sensitive while cells with GI₅₀>1 μM are designated as resistant. Mutation analysis is performed based on the Catalogue of Somatic Mutations in Cancer (COSMIC) v41. The Fisher's exact test is applied. A p<0.05 is considered statistically significant.

### In Vivo Studies

All experimental procedures are performed according to the Guide for the Care and Use of Laboratory Animals.

For acute pharmacodynamic studies, selected cells such as AN3CA cells (5×10⁶) or BT-474 cells (10×10⁶) are inoculated subcutaneously into female NCr nu/nu mice three weeks prior to compound administration. Compound is orally dosed at selected doses for AN3CA bearing mice (n=8) and 200 mg/kg for BT-474 bearing mice (n=4 for Vehicle: n=5 for ARQ 092). Tumor tissues are collected after 1 or 8 hours for AN3CA or 2 hours for BT-474.

For efficacy studies, AN3CA cells (5×10⁶) are inoculated into female NCr nu/nu mice. When tumors reached a size of 250 mm³, mice are randomly grouped into 5 groups. Vehicle (10% DMA in water), or compound are dosed orally daily or every other day. KPL-4 cells (1.5×10⁷) are inoculated subcutaneously into female nude mice (BALB/cAJc1-nu/nu). When the average size of the tumor reached over 100 mm³, mice are randomly divided into 6 groups: vehicle (0.5% w/v % methyl cellulose 400 cP solution) and compound at 10, 20, 40, 80, or 120 mg/kg. For each group, compound is orally dosed daily for 8 days. ZR-75-1 tumor fragments are inoculated into female NOD/scid mice. When tumors reached a size of 300 mm³, untreated control, or compound at 20 mg/kg or 40 mg/kg is orally dosed on a schedule of 5 days on, 2 days off and 4 days on.

The endometrial PDX tumor model (ST1061) is developed from a 43 year old African American female with chemotherapy naïve endometrial adenocarcinoma harboring an AKT1-E17K mutation. In this study; athymic nude mice (Charles River Labs) are subcutaneously implanted with tumor fragments and the study initiated seven days later at a mean tumor volume of approximately 200 mm³. Mice (n=10 per dose group) are treated with vehicle or drug. At these levels, animals are dosed once daily for 20 days on a schedule of 5 days of dosing followed by 2 days with no dosing.

A melanoma PDX tumor model (ST052C) that contained BRAF V600E and PIK3CA (H1047R) mutations and exhibited acquired vemurafenib-resistance (originating from a 60 year old Caucasian male) is used in this study. Athymic nude mice (Charles River Laboratories, Wilmington, MA) are subcutaneously implanted with tumor fragments and the study is initiated seven days later when the mean tumor volume is approximately 140 mm³. Mice (n=5 per dose group) are treated with vehicle or compound.

The tumor length and width (mm) are measured using a digital caliper. The estimated tumor volume (mm³) is calculated according to the following formula: 0.5×(tumor length)×(tumor width) 2, and reported as the mean±SEM. The tumor growth inhibition (TGI) is determined and the vehicle group is designated as 100% tumor growth.

### AKT Ubiquitination Assay

For each assay condition. 5.0×10⁶ KU-19-19 cells were seeded in 10 cm dish grown in RPMI (Invitrogen. Carlsbad. CA. USA) supplemented with 10% fetal bovine serum. Cells were incubated overnight at 37° C. in a humidified atmosphere containing 5% CO2. 5% CO2. Next day, media was removed and cells were rinsed with 0.1% FBS cell culture media. Then 10 mL of 0.1% FBS+RPMI containing 3 uM of compound was added to each plate and treated with compound for 6 hrs. Cells were washed 3× with PBS and placed on ice. 900 μL of lysis buffer (50 mM Tris-HCl pH 7.4, 150 mM NaCl. 1% NP-40, 1% Sodium deoxycholate. 2 mM EDTA, 10% Glycerol, 5 mM Iodoacetamide (dissolved in ddH2O, MCE, Cat #HY-34477), 1×HALT protease\phosphatase inhibitor (Pierce. Cat #78440), 1×PMSF (Sigma. Cat #93482-50ML-F), 5 mM 1.10 Phenanthroline (in DMSO, MCE, Cat #HY—W004544), 25 uM MG-132 (in DMSO) (MCE. Cat #HY-13259), 50 μM PR-619 (make it in DMSO) (MCE. Cat #HY-13814) and 1× Benzonase (Sigma. Cat #E8263) was added and incubated on ice for 3-5 min and then scraped and collected. Lysates were incubated on ice for 1 hr and vortexed every 15 minutes before spinning down at 14 000 g for 10 minutes. Supernatants were collected and flash frozen in Liquid nitrogen for 10 minutes and stored at −80 C until further used. Adjust volume of samples with lysis buffer to obtain 1 mg in total volume of 1 mL. Perform TUBES pulldown following manufacturer's procedure using TUBE1 (high capacity magnetic beads) (Cat #UM-0501M-1000. LifeSensors). Briefly, wash 150 μl beads per sample with 12× beads volume of PBST (0.1% Tweed-20) three times. Add 1 mL of protein lysate to beads and incubate for 4 hours rotating at 4 C. Wash 4× with 10× beads volume of lysis buffer and rotate for 3 minutes, then collect beads with magnet for 3 minutes before collecting buffer. Wash 1× with 5× beads volumes of PBS-T. Wash TUBEs with 100 μl wash buffer for 5 minutes on thermomixer set at 1400 rpm and 25 C. TUBE washing buffer/TUBE Elution buffer/TUBE Neutralization buffer (Cat #UM411. LifeSensors) Elute with 18 μl of TUBE elution buffer for 15 minutes on thermomixer set at 1400 rpm at 25 C, and then collect eluate and add 2 μl of neutralization buffer. Load 18 μl of pulldown sample and 10 μg of input samples onto gel for western blot using standard methods with following antibodies: AKT (pan) (C67E7) (CST. Cat #4691S). β-Actin antibody (Cat #A1978, sigma) (Dilution. 1:2000) Anti-rabbit IgG. HRP-linked Antibody (CST. Cat #7074S) (Dilution, 1:2000)

Cell Titer Glo (CTG) assay

| | | Seeding |
|---|---|---|
| | | Density |
| Cell Line | Culture Medium | (cells/well) |
| KU1919 | RPMI1640 + 10% FBS | 200 |
| MDA-MB-231 | RPMI1640 + 10% FBS | 800 |
| MDA-MB-468 | RPMI1630 + 10% FBS | 2000 |

Seed cells in 40 μL of cell culture media into each well of 384-well cell culture plate, spin them at room temperature at 1,000 RPM for 1 minute and then transfer the plates into 37° C. 5% CO₂ incubator overnight. Next day, treat cells with 40 nL of diluted compound and return to incubator. After compound treatment for 3 days, Add 30 μL of CellTiter-Glo reagent (Promega) into each well to be detected. Then place the plates at room temperature for 30 min followed by reading on EnVision. Inhibition activity calculated as % DMSO=100×(LumSample-Lum low control)/(Lum high control-Lum low control) where high control is from cells treated with 0.1% DMSO and Low control obtained from PBS only. Use XLFit (equation 201) to calculate IC50 (A+ ((B−A)/(1×((x/C)\hat{ }D)))) where A: Bottom, B: Top, C: IC50, D: Slope. Table 4, below, shows the results of the Cell Titer Glo (CTG) assay.

AKT plasma membrane translocation assay

Seed 1.5×10⁶ KU-19-19 cells in 10 cm dish. Transfect cells at 70% cell confluency with 5 ug AKTIE17K-SmBIT and 0.5 ug LCK1-LgBiT with Jet-Optimus transfection reagent. Incubate for 4-5 hours at 37c 5% CO₂ TC incubator. Split and seed 10K cells in 40 ul media per well in 384 well plate and incubate over night at 37° C. CO₂ free TC incubator. Add 10 ul of 5× Vivazine substrate (prepared in CO₂ independent medium) to cells and shake palte to mix for 30 seconds. Read Luminescence for 100 minutes until signal is stable (1 read per minute). Add 50 nl/well of 3 uM compound stock to cells. Read plate by Envision for up to 1 hr, Table 3, below, shows the results of the AKT plasma membrane translocation assay.

TABLE 3
Profiling of compounds in ubiquitination and
plasma membrane translocation assay

| | | Reduced AKT1 |
|---|---|---|
| | Reduced AKT | E17 plasma |
| | ubiquitination | membrane |
| | relative | translocation |
| I-# | to PBM | relative to PBM |
| I-17 | Yes | Yes |
| I-46 | Yes | Yes |
| I-312 | Yes | Yes |
| I-254 | Yes | Yes |
| I-242 | Yes | Yes |

Table 5 shows the results of the cell proliferation assay in KU1919.1, MDAMB231.1, and MDAMB468.1 cells. The letter codes for the cell proliferation assay in KU1919.1, MDAMB231.1, and MDAMB468.1 cells include: A (<500 nM), B (≥500-1000 nM), C (>1000-5000 nM), D (>5000-10000 nM), E (>10000 nM), and “-” is not tested.

TABLE 4
Proliferation Assay

| | AVE CTG | AVE CTG | AVE CTG |
|---|---|---|---|
| | Proliferation | Proliferation | Proliferation |
| | KU1919.1 | MDAMB231.1 | MDAMB468.1 |
| I-# | 72.0 hrs. | 72 hrs. | 72.0 hrs. |
| GDC-0068 | E | E | E |
| AZD-5363 | E | E | E |
| I-17 | C | C | C |
| I-171 | B | — | C |
| I-172 | D | — | D |
| I-173 | C | — | C |
| I-174 | C | — | C |
| I-214 | C | — | C |
| I-216 | C | — | C |
| I-217 | C | — | C |
| I-219 | B | C | C |
| I-328 | E | — | E |
| I-167 | E | — | E |
| I-197 | D | — | C |
| I-200 | C | — | C |
| I-175 | C | — | C |
| I-215 | E | — | E |
| I-201 | B | C | C |
| I-211 | B | C | C |
| I-196 | C | D | D |
| I-183 | D | — | D |
| I-194 | D | — | D |
| I-304 | C | — | C |
| I-301 | C | — | C |
| I-312 | C | C | C |
| I-235 | E | E | E |
| I-178 | C | — | — |
| I-204 | C | C | C |
| I-165 | E | — | E |
| I-184 | D | — | E |
| I-195 | E | — | E |
| I-305 | C | C | C |
| I-206 | E | E | E |
| I-207 | E | E | E |
| I-208 | C | C | C |
| I-209 | D | D | D |
| I-213 | C | C | C |
| I-306 | C | C | C |
| I-223 | C | — | C |
| I-225 | C | — | C |
| I-254 | C | C | C |
| I-255 | E | — | E |
| I-256 | D | — | D |
| I-257 | E | — | E |
| I-203 | D | — | D |
| I-205 | D | — | E |
| I-224 | C | C | C |
| I-240 | C | C | C |
| I-242 | B | C | C |
| I-248 | C | D | D |
| I-252 | D | E | D |
| I-253 | C | — | C |
| I-258 | B | C | C |
| I-259 | C | C | C |
| I-226 | B | C | C |
| I-227 | C | C | C |
| I-166 | C | — | — |
| I-241 | C | D | C |
| I-246 | C | — | — |
| I-309 | C | — | — |
| I-237 | D | — | — |
| I-313 | C | — | — |
| I-229 | C | — | — |
| I-232 | E | — | — |
| I-326 | D | — | — |
| I-220 | C | — | — |
| I-222 | C | — | — |
| I-231 | C | — | — |
| I-308 | C | — | — |
| I-233 | D | — | — |
| I-310 | C | D | C |
| I-311 | B | — | — |
| I-250 | C | — | — |
| I-314 | C | — | — |
| I-234 | E | — | — |
| I-239 | E | — | — |
| I-221 | C | — | — |
| I-243 | C | — | — |
| I-244 | C | — | — |
| I-245 | B | — | — |
| I-249 | C | — | — |
| I-251 | B | — | — |
| I-315 | C | E | C |
| I-230 | C | C | C |
| I-260 | C | — | — |
| I-263 | C | — | — |
| I-261 | C | C | C |
| I-264 | C | — | — |
| I-247 | C | — | — |
| I-270 | C | — | — |
| I-284 | C | — | — |
| I-228 | E | — | — |
| I-236 | B | — | — |
| I-238 | E | — | — |
| I-262 | B | C | C |
| I-265 | C | — | — |
| I-266 | C | — | — |
| I-316 | C | — | — |
| I-272 | C | — | — |
| I-276 | C | — | — |
| I-277 | A | A | A |
| I-278 | B | C | C |
| I-279 | B | C | C |
| I-286 | C | — | — |
| I-281 | C | A | C |
| I-273 | B | — | — |
| I-274 | B | — | — |
| I-275 | B | — | — |
| I-285 | E | — | — |
| I-287 | E | — | — |
| I-280 | C | — | — |
| I-282 | C | C | C |
| I-283 | C | C | C |
| I-291 | C | — | — |
| I-292 | C | — | — |
| I-293 | C | — | — |
| I-294 | C | — | — |
| I-295 | C | — | — |
| I-296 | E | — | — |
| I-297 | D | — | — |
| I-298 | C | — | — |
| I-288 | C | — | — |
| I-290 | C | — | — |
| I-289 | C | — | — |

1. A compound of formula I, 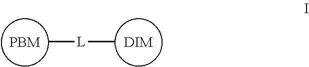
or a pharmaceutically acceptable salt thereof, wherein:
PBM is a protein binding moiety capable of binding Akt;
L is a bivalent moiety that connects PBM to DIM; and
DIM is a deubiquitination inducing moiety.

2. (canceled)

3. The compound of claim 1, wherein PBM is an Akt binding moiety thereby forming a compound of formula I-a′: 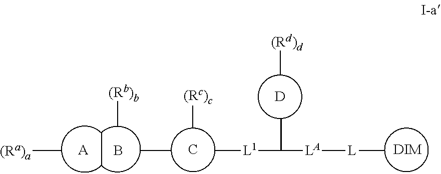
or a pharmaceutically acceptable salt thereof, wherein L and DIM are as defined above and described herein, and wherein:
Ring A and Ring B are independently fused rings selected from benzo, a 4-7 membered saturated or partially unsaturated carbocyclyl, a 4-7 membered saturated or partially unsaturated heterocyclyl with 1-3 heteroatoms independently selected from nitrogen, oxygen, and sulfur, and a 5-6 membered heteroaryl with 1-4 heteroatoms independently selected from nitrogen, oxygen and sulfur;
Ring C is a ring selected from phenylenyl, a 4-7 membered saturated or partially unsaturated carbocyclylenyl, a 8-10 membered saturated or partially unsaturated bridged bicyclic carbocyclylenyl, a 4-7 membered saturated or partially unsaturated heterocyclylenyl with 1-3 heteroatoms independently selected from nitrogen, oxygen, and sulfur, a 8-10 membered saturated or partially unsaturated bridged bicyclic heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
Ring D is a ring selected from phenyl, a 4-7 membered saturated or partially unsaturated carbocyclyl, a 4-7 membered saturated or partially unsaturated heterocyclyl with 1-3 heteroatoms independently selected from nitrogen, oxygen, and sulfur, and a 5-6 membered heteroaryl with 1-4 heteroatoms independently selected from nitrogen, oxygen and sulfur;
L¹ is a covalent bond or a bivalent, saturated or partially unsaturated, straight or branched C₁₋₅ hydrocarbon chain, wherein 0-3 methylene units of L¹ or L^{A} are independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, or —OC(O)NR—, —NRC(O)O—;
L^{A} is a covalent bond or a bivalent, saturated or partially unsaturated, straight or branched C₁₋₅ hydrocarbon chain, wherein 0-3 methylene units of L¹ or L^{A} are independently replaced by -Cy-, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, or —OC(O)NR—, —NRC(O)O—;
each -Cy- is independently an optionally substituted bivalent ring selected from phenylenyl, an 8-10 membered bicyclic arylenyl, a 4-7 membered saturated or partially unsaturated carbocyclylenyl, a 6-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 6-11 membered saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, and an 8-10 membered bicyclic heteroarylenyl having 1-5 heteroatoms independently selected from nitrogen, oxygen, and sulfur;
each R^{a}, R^{b}, R^{c}, and R^{d} is independently selected from hydrogen, R^{A}, halogen, —CN, —NO₂, —OR, —SR, —NR₂, —S(O)₂R, —S(O)₂NR₂, —S(O)R, —C(O)R, —C(O)OR, —C(O)NR₂, —C(O)NROR, —OC(O)R, —OC(O)NR₂, —NRC(O)OR, —NRC(O)R, —NRC(O)N(R)₂, and —NRS(O)₂R;
each R^{A} is independently an optionally substituted group selected from C₁₋₆ aliphatic, phenyl, a 3-12 membered saturated or partially unsaturated monocyclic, bicyclic, bridged bicyclic or spirocyclic carbocyclyl or heterocyclyl with 1-3 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or;
two R groups on the same nitrogen are optionally taken together with their intervening atoms to form a 4-7 membered saturated, partially unsaturated, or heteroaryl ring having 0-3 heteroatoms, in addition to the nitrogen atom to which they are attached, independently selected from nitrogen, oxygen, and sulfur;
a, b, c, and d are independently 0, 1, 2, 3, or 4;
L is a covalent bond or a bivalent, saturated or partially unsaturated, straight or branched C₁₋₅₀ hydrocarbon chain, wherein 0-6 methylene units of L are independently replaced by -Cy-, —O—, —NR—, —SiR₂—, —Si(OH)R—, —Si (OH)₂—, —P(O)OR—, —P(O)R—, —P(O)NR₂—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, —NRC(O)O—, 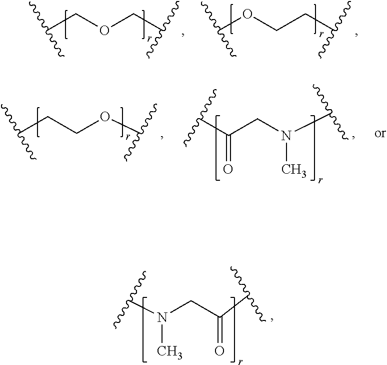
wherein:
each -Cy- is independently an optionally substituted bivalent ring selected from phenylenyl, an 8-10 membered bicyclic arylenyl, a 4-7 membered saturated or partially unsaturated carbocyclylenyl, a 6-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 6-11 membered saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, and an 8-10 membered bicyclic heteroarylenyl having 1-5 heteroatoms independently selected from nitrogen, oxygen, and sulfur;
r is 0, 1, 2, 3, 4, 5, 6, 7, 8, 9, or 10; and L is a covalent bond or a bivalent, saturated or partially unsaturated, straight or branched C₁₋₅₀ hydrocarbon chain, wherein 0-6 methylene units of L are independently replaced by -Cy-, —O—, —NR—, —SiR₂—, —Si(OH)R—, —Si (OH)₂—, —P(O)OR—, —P(O)R—, —P(O)NR₂—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, —NRC(O)O—, 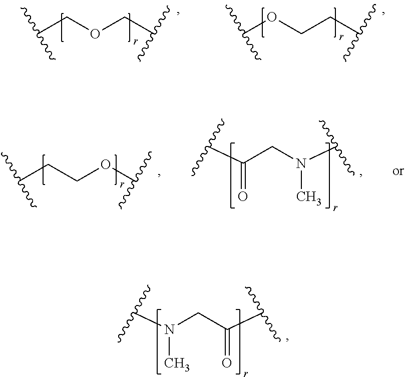
wherein:
each -Cy- is independently an optionally substituted bivalent ring selected from phenylenyl, an 8-10 membered bicyclic arylenyl, a 4-7 membered saturated or partially unsaturated carbocyclylenyl, a 6-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 6-11 membered saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, and an 8-10 membered bicyclic heteroarylenyl having 1-5 heteroatoms independently selected from nitrogen, oxygen, and sulfur; and
r is 0, 1, 2, 3, 4, 5, 6, 7, 8, 9, or 10.

4-7. (canceled)

8. The compound of claim 3, wherein ring D is a phenyl.

9-10. (canceled)

11. The compound of claim 3, wherein ring C is 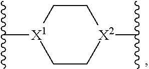
wherein X¹ and X² are independently carbon or nitrogen.

12-23. (canceled)

24. The compound of claim 3, wherein DIM is a deubiquitinase binding moiety (DBM), thereby forming:
a compound of formula I-bb: 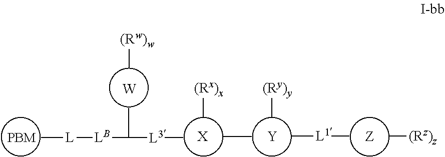
or a pharmaceutically acceptable salt thereof, wherein:
Ring Z is a ring selected from phenyl, a 4-7 membered saturated or partially unsaturated carbocyclic ring, a 4-7 membered saturated or partially unsaturated heterocyclic ring with 1-3 heteroatoms independently selected from nitrogen, oxygen, and sulfur, and a 5-6 membered monocyclic heteroaromatic ring with 1-4 heteroatoms independently selected from nitrogen, oxygen and sulfur;
Ring Y is a ring selected from phenylenyl, a 4-7 membered saturated or partially unsaturated carbocyclylenyl, a 4-7 membered saturated or partially unsaturated heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
Ring X is a ring selected from phenylenyl, a 4-7 membered saturated or partially unsaturated carbocyclylenyl, a 4-7 membered saturated or partially unsaturated heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
Ring W is a ring selected from phenylenyl, a 4-7 membered saturated or partially unsaturated carbocyclylenyl, a 4-7 membered saturated or partially unsaturated heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
each L^{B}, L^{1′}, and L^{3′} is independently a covalent bond or a bivalent, saturated or partially unsaturated, straight or branched C₁₋₅ hydrocarbon chain, wherein 0-3 methylene units of L^{B}, L^{1′}, or L^{3′} are independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, or —OC(O)NR—, —NRC(O)O—;
each R^{w}, R^{x}, R^{y}, and R^{z} is independently selected from hydrogen, R^{B}, halogen, —CN, —NO₂, —OR, —SR, —NR₂, —S(O)₂R, —S(O)₂NR₂, —S(O)R, —C(O)R, —C(O)OR, —C(O)NR₂, —C(O)NROR, —OC(O)R, —OC(O)NR₂, —NRC(O)OR, —NRC(O)R, —NRC(O)N(R)₂, and —NRS(O)₂R;
each R^{B} is independently an optionally substituted group selected from C₁₋₆ aliphatic, phenyl, a 3-12 membered saturated or partially unsaturated monocyclic, bicyclic, bridged bicyclic or spirocyclic carbocyclyl or heterocyclyl with 1-3 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or;
two R groups on the same nitrogen are optionally taken together with their intervening atoms to form a 4-7 membered saturated, partially unsaturated, or heteroaryl ring having 0-3 heteroatoms, in addition to the nitrogen atom to which they are attached, independently selected from nitrogen, oxygen, and sulfur; and
w, x, y, and z are independently 0, 1, 2, 3, or 4; or
a compound of formula I-bb′: 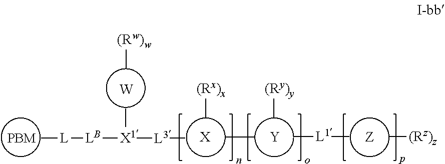
or a pharmaceutically acceptable salt thereof, wherein:
Ring Z is a ring selected from phenyl, a 3-7 membered saturated or partially unsaturated monocyclic, bicyclic, bridged bicyclic or spirocyclic carbocyclic ring, a 3-7 membered saturated or partially unsaturated monocyclic, bicyclic, bridged bicyclic or spirocyclic heterocyclic ring with 1-3 heteroatoms independently selected from nitrogen, oxygen, and sulfur, and a 5-6 membered heteroaromatic ring with 1-4 heteroatoms independently selected from nitrogen, oxygen and sulfur;
Ring Y is a ring selected from phenylenyl, a 4-8 membered saturated or partially unsaturated carbocyclylenyl, a 4-8 membered saturated or partially unsaturated monocyclic, bicyclic, bridged bicyclic or spirocyclic heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
Ring X is a ring selected from phenylenyl, a 4-7 membered saturated or partially unsaturated carbocyclylenyl, a 4-7 membered saturated or partially unsaturated heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
Ring W is a ring selected from phenylenyl, a 4-7 membered saturated or partially unsaturated monocyclic, bicyclic, bridged bicyclic or spirocyclic carbocyclylenyl, a 4-7 membered saturated or partially unsaturated monocyclic, bicyclic, bridged bicyclic or spirocyclic heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
each L^{B}, L^{1′}, and L^{3′} is independently a covalent bond or an optionally substituted bivalent, saturated or partially unsaturated, straight or branched C₁₋₅ hydrocarbon chain, wherein 0-3 methylene units of L^{B}, L^{1′}, or L^{3′} are independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, or —OC(O)NR—, —NRC(O)O—;
each R^{w}, R^{x}, R^{y}, and R^{z} is independently selected from hydrogen, R^{B}, halogen, —CN, —NO₂, —OR, —SR, —NR₂, —S(O)₂R, —S(O)₂NR₂, —S(O)R, —C(O)R, —C(O)OR, —C(O)NR₂, —C(O)NROR, —OC(O)R, —OC(O)NR₂, —NRC(O)OR, —NRC(O)R, —NRC(O)N(R)₂, and —NRS(O)₂R;
each R^{B} is independently an optionally substituted group selected from C₁₋₆ aliphatic, phenyl, a 3-12 membered saturated or partially unsaturated monocyclic, bicyclic, bridged bicyclic or spirocyclic carbocyclyl or heterocyclyl with 1-3 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or;
two R groups on the same nitrogen are optionally taken together with their intervening atoms to form a 4-7 membered saturated, partially unsaturated, or heteroaryl ring having 0-3 heteroatoms, in addition to the nitrogen atom to which they are attached, independently selected from nitrogen, oxygen, and sulfur;
X¹ is carbon or nitrogen;
m, n, o, and p are independently 0 or 1; and
w, x, y, and z are independently 0, 1, 2, 3, or 4; or
a compound of formula I-cc: 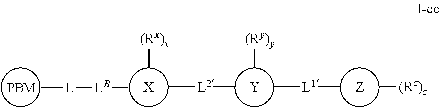
or a pharmaceutically acceptable salt thereof, wherein:
Ring Z is a ring selected from phenyl, a 4-7 membered saturated or partially unsaturated carbocyclic ring, a 4-7 membered saturated or partially unsaturated heterocyclic ring with 1-3 heteroatoms independently selected from nitrogen, oxygen, and sulfur, and a 5-6 membered heteroaromatic ring with 1-4 heteroatoms independently selected from nitrogen, oxygen and sulfur;
Ring Y is a ring selected from phenylenyl, a 4-7 membered saturated or partially unsaturated carbocyclylenyl, a 4-7 membered saturated or partially unsaturated heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
Ring X is a ring selected from phenylenyl, a 4-7 membered saturated or partially unsaturated carbocyclylenyl, a 4-7 membered saturated or partially unsaturated heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
each of L^{B}, L^{1′}, and L² is independently a covalent bond or a bivalent, saturated or partially unsaturated, straight or branched C₁₋₅ hydrocarbon chain, wherein 0-3 methylene units of L^{B}, L^{1′}, or L^{2′} are independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, or —OC(O)NR—, —NRC(O)O—;
each R^{x}, R^{y}, and R^{z} is independently selected from hydrogen, R^{B}, halogen, CN, —NO₂, —OR, —SR, —NR₂, —S(O)₂R, —S(O)₂NR₂, —S(O)R, —C(O)R, —C(O)OR, —C(O)NR₂, —C(O)NROR, —OC(O)R, —OC(O)NR₂, —NRC(O)OR, —NRC(O)R, —NRC(O)N(R)₂, and —NRS(O)₂R;
each R^{B} is independently an optionally substituted group selected from C₁₋₆ aliphatic, phenyl, a 3-12 membered saturated or partially unsaturated monocyclic, bicyclic, bridged bicyclic or spirocyclic carbocyclyl or heterocyclyl with 1-3 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or;
two R groups on the same nitrogen are optionally taken together with their intervening atoms to form a 4-7 membered saturated, partially unsaturated, or heteroaryl ring having 0-3 heteroatoms, in addition to the nitrogen atom to which they are attached, independently selected from nitrogen, oxygen, and sulfur; and
x, y, and z are independently 0, 1, 2, 3, or 4; or
a compound of formula I-cc′: 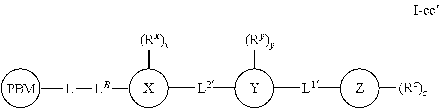
or a pharmaceutically acceptable salt thereof, wherein:
Ring Z is a ring selected from phenyl, a 3-7 membered saturated or partially unsaturated monocyclic, bicyclic, bridged bicyclic or spirocyclic carbocyclic ring, a 3-7 membered saturated or partially unsaturated monocyclic, bicyclic, bridged bicyclic or spirocyclic heterocyclic ring with 1-3 heteroatoms independently selected from nitrogen, oxygen, and sulfur, and a 5-6 membered heteroaromatic ring with 1-4 heteroatoms independently selected from nitrogen, oxygen and sulfur and an 8-10 membered bicyclic heteroaromatic ring having 1-5 heteroatoms independently selected from nitrogen, oxygen, or sulfur;
Ring Y is a ring selected from phenylenyl, a 4-7 membered saturated or partially unsaturated carbocyclylenyl, a 4-8 membered saturated or partially unsaturated monocyclic, bicyclic, bridged bicyclic or spirocyclic heterocyclylenyl with 1-3 heteroatoms independently selected from nitrogen, oxygen, and sulfur, a 5-6 membered heteroarylenyl with 1-4 heteroatoms independently selected from nitrogen, oxygen and sulfur and an 8-10 membered bicyclic heterocyclylenyl having 1-5 heteroatoms independently selected from nitrogen, oxygen, or sulfur;
Ring X is a ring selected from phenylenyl, a 4-7 membered saturated or partially unsaturated carbocyclylenyl, a 4-7 membered saturated or partially unsaturated heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
each of L^{B}, L^{1′} and L^{2′} is independently a covalent bond or an optionally substituted bivalent, saturated or partially unsaturated, straight or branched C₁₋₅ hydrocarbon chain, wherein 0-3 methylene units of L^{B}, L^{1′}, or L^{2′} are independently replaced by -Cy′-, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, or —OC(O)NR—, —NRC(O)O—;
each R^{x}, R^{y}, and R^{z} is independently selected from hydrogen, R^{B}, halogen, CN, —NO₂, —OR, —SR, —NR₂, —S(O)₂R, —S(O)₂NR₂, —S(O)R, —C(O)R, —C(O)OR, —C(O)NR₂, —C(O)NROR, —OC(O)R, —OC(O)NR₂, —NRC(O)OR, —NRC(O)R, —NRC(O)N(R)₂, and —NRS(O)₂R;
each R^{B} is independently an optionally substituted group selected from C₁₋₆ aliphatic, phenyl, a 3-12 membered saturated or partially unsaturated monocyclic, bicyclic, bridged bicyclic or spirocyclic carbocyclyl or heterocyclyl with 1-3 heteroatoms independently selected from nitrogen, oxygen, and sulfur, and a 5-6 membered heteroaryl ring having 1-4 heteroatoms independently selected from nitrogen, oxygen, and sulfur;
each -Cy′- is independently an optionally substituted bivalent ring selected from phenylenyl, an 8-10 membered bicyclic arylenyl, a 3-11 membered saturated or partially unsaturated monocyclic, bicyclic, bridged bicyclic or spirocyclic carbocyclylenyl, a 3-11 membered saturated or partially unsaturated monocyclic, bicyclic, bridged bicyclic or spirocyclic heterocyclylenyl having 1-3 heteroatoms independently selected from nitrogen, oxygen, and sulfur, a 5-6 membered heteroarylenyl having 1-4 heteroatoms independently selected from nitrogen, oxygen, and sulfur, and an 8-10 membered bicyclic heteroarylenyl having 1-5 heteroatoms independently selected from nitrogen, oxygen, and sulfur;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or;
two R groups on the same nitrogen are optionally taken together with their intervening atoms to form a 4-7 membered saturated, partially unsaturated, or heteroaryl ring having 0-3 heteroatoms, in addition to the nitrogen atom to which they are attached, independently selected from nitrogen, oxygen, and sulfur; and
x, y, and z are independently 0, 1, 2, 3, or 4; or
a compound of formula I-ff: 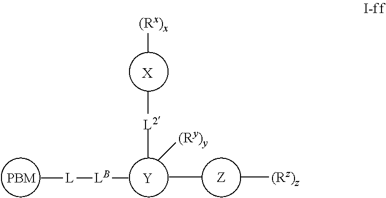
or a pharmaceutically acceptable salt thereof, wherein:
Ring Z is a ring selected from phenyl, a 4-7 membered saturated or partially unsaturated carbocyclic ring, a 4-7 membered saturated or partially unsaturated heterocyclic ring with 1-3 heteroatoms independently selected from nitrogen, oxygen, and sulfur, and a 5-6 membered heteroaromatic ring with 1-4 heteroatoms independently selected from nitrogen, oxygen and sulfur;
Ring Y is a ring selected from phenylenyl, a 4-7 membered saturated or partially unsaturated carbocyclylenyl, a 4-7 membered saturated or partially unsaturated heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
Ring X is a ring selected from phenylenyl, a 4-7 membered saturated or partially unsaturated carbocyclylenyl, a 4-7 membered saturated or partially unsaturated heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
each of L^{B} and L^{2′} is independently a covalent bond or a covalent bond or a bivalent, saturated or partially unsaturated, straight or branched C₁₋₅ hydrocarbon chain, wherein 0-3 methylene units of L^{B} or L^{2′} are independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, or —OC(O)NR—, —NRC(O)O—;
each R^{x}, R^{y}, and R^{z} is independently selected from hydrogen, R^{B}, halogen, —CN, —NO₂, —OR, —SR, —NR₂, —S(O)₂R, —S(O)₂NR₂, —S(O)R, —C(O)R, —C(O)OR, —C(O)NR₂, —C(O)NROR, —OC(O)R, —OC(O)NR₂, —NRC(O)OR, —NRC(O)R, —NRC(O)N(R)₂, and —NRS(O)₂R;
each R^{B} is independently an optionally substituted group selected from C₁₋₆ aliphatic, phenyl, a 3-12 membered saturated or partially unsaturated monocyclic, bicyclic, bridged bicyclic or spirocyclic carbocyclyl or heterocyclyl with 1-3 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or;
two R groups on the same nitrogen are optionally taken together with their intervening atoms to form a 4-7 membered saturated, partially unsaturated, or heteroaryl ring having 0-3 heteroatoms, in addition to the nitrogen atom to which they are attached, independently selected from nitrogen, oxygen, and sulfur; and
x, y, and z are independently 0, 1, 2, 3, or 4; or
a compound of formula I-ff′: 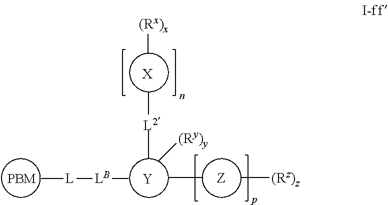
or a pharmaceutically acceptable salt thereof, wherein:
Ring Z is a ring selected from phenyl, a 4-9 membered saturated or partially unsaturated monocyclic, bicyclic, bridged bicyclic or spirocyclic carbocyclic ring, a 4-9 membered saturated or partially unsaturated monocyclic, bicyclic, bridged bicyclic or spirocyclic heterocyclic ring with 1-3 heteroatoms independently selected from nitrogen, oxygen, and sulfur, a 5-6 membered heteroaromatic ring with 1-4 heteroatoms independently selected from nitrogen, oxygen and sulfur and an 8-10 membered bicyclic heteroaromatic ring having 1-5 heteroatoms independently selected from nitrogen, oxygen, or sulfur;
Ring Y is a ring selected from phenylenyl, a 4-7 membered saturated or partially unsaturated carbocyclylenyl, a 4-7 membered saturated or partially unsaturated heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
Ring X is a ring selected from phenylenyl, a 3-9 membered saturated or partially unsaturated monocyclic, bicyclic, bridged bicyclic or spirocyclic carbocyclylenyl, a 3-9 membered saturated or partially unsaturated monocyclic, bicyclic, bridged bicyclic or spirocyclic heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
each of L^{B} and L^{2′} is independently a covalent bond or an optionally substituted bivalent, saturated or partially unsaturated, straight or branched C₁₋₅ hydrocarbon chain, wherein 0-3 methylene units of L^{B} or L^{2′} are independently replaced by -Cy′-, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, or —OC(O)NR—, —NRC(O)O—;
each R^{x}, R^{y}, and R^{z} is independently selected from hydrogen, R^{B}, halogen, —CN, —NO₂, —OR, —SR, —NR₂, —S(O)₂R, —S(O)₂NR₂, —S(O)R, —C(O)R, —C(O)OR, —C(O)NR₂, —C(O)NROR, —OC(O)R, —OC(O)NR₂, —NRC(O)OR, —NRC(O)R, —NRC(O)N(R)₂, and —NRS(O)₂R;
each R^{B} is independently an optionally substituted group selected from C₁₋₆ aliphatic, phenyl, a 3-12 membered saturated or partially unsaturated monocyclic, bicyclic, bridged bicyclic or spirocyclic carbocyclyl or heterocyclyl with 1-3 heteroatoms independently selected from nitrogen, oxygen, and sulfur, and a 5-6 membered heteroaryl ring having 1-4 heteroatoms independently selected from nitrogen, oxygen, and sulfur;
each -Cy′- is independently an optionally substituted bivalent ring selected from phenylenyl, an 8-10 membered bicyclic arylenyl, a 3-11 membered saturated or partially unsaturated monocyclic, bicyclic, bridged bicyclic or spirocyclic carbocyclylenyl, a 3-11 membered saturated or partially unsaturated monocyclic, bicyclic, bridged bicyclic or spirocyclic heterocyclylenyl having 1-3 heteroatoms independently selected from nitrogen, oxygen, and sulfur, a 5-6 membered heteroarylenyl having 1-4 heteroatoms independently selected from nitrogen, oxygen, and sulfur, and an 8-10 membered bicyclic heteroarylenyl having 1-5 heteroatoms independently selected from nitrogen, oxygen, and sulfur;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or;
two R groups on the same nitrogen are optionally taken together with their intervening atoms to form a 4-7 membered saturated, partially unsaturated, or heteroaryl ring having 0-3 heteroatoms, in addition to the nitrogen atom to which they are attached, independently selected from nitrogen, oxygen, and sulfur;
n and p are independently 0 or 1; and
x, y, and z are independently 0, 1, 2, 3, or 4; or
a compound of formula I-gg: 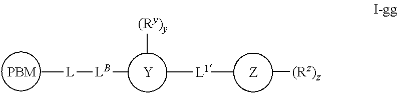
or a pharmaceutically acceptable salt thereof, wherein:
Ring Z is a ring selected from phenyl, a 4-7 membered saturated or partially unsaturated carbocyclic ring, a 4-7 membered saturated or partially unsaturated heterocyclic ring with 1-3 heteroatoms independently selected from nitrogen, oxygen, and sulfur, and a 5-6 membered heteroaromatic ring with 1-4 heteroatoms independently selected from nitrogen, oxygen and sulfur;
Ring Y is a ring selected from phenylenyl, a 4-7 membered saturated or partially unsaturated carbocyclylenyl, a 4-7 membered saturated or partially unsaturated heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
each of L^{B} and L^{1′} is independently a covalent bond or a covalent bond or a bivalent, saturated or partially unsaturated, straight or branched C₁₋₅ hydrocarbon chain, wherein 0-3 methylene units of L^{B} or L^{1′} are independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, or —OC(O)NR—, —NRC(O)O—;
each R^{y}, and R^{z} is independently selected from hydrogen, R^{B}, halogen, CN, —NO₂, —OR, —SR, —NR₂, —S(O)₂R, —S(O)₂NR₂, —S(O)R, —C(O)R, —C(O)OR, —C(O)NR₂, —C(O)NROR, —OC(O)R, —OC(O)NR₂, —NRC(O)OR, —NRC(O)R, —NRC(O)N(R)₂, and —NRS(O)₂R;
each R^{B} is independently an optionally substituted group selected from C₁₋₆ aliphatic, phenyl, a 3-12 membered saturated or partially unsaturated monocyclic, bicyclic, bridged bicyclic or spirocyclic carbocyclyl or heterocyclyl with 1-3 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or;
two R groups on the same nitrogen are optionally taken together with their intervening atoms to form a 4-7 membered saturated, partially unsaturated, or heteroaryl ring having 0-3 heteroatoms, in addition to the nitrogen atom to which they are attached, independently selected from nitrogen, oxygen, and sulfur; and
y and z are independently 0, 1, 2, 3, or 4; or
a compound of formula I-hh: 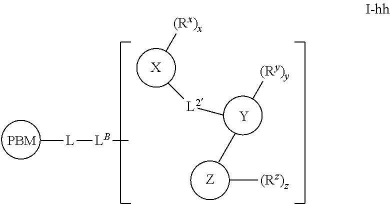
or a pharmaceutically acceptable salt thereof, wherein:
Ring Z is a ring selected from phenyl, a 4-7 membered saturated or partially unsaturated carbocyclic ring, a 4-7 membered saturated or partially unsaturated heterocyclic ring with 1-3 heteroatoms independently selected from nitrogen, oxygen, and sulfur, a 5-6 membered heteroaromatic ring with 1-4 heteroatoms independently selected from nitrogen, oxygen and sulfur, and an 8-10 membered bicyclic heteroaromatic ring having 1-5 heteroatoms independently selected from nitrogen, oxygen, or sulfur;
Ring Y is a ring selected from phenylenyl, a 4-7 membered saturated or partially unsaturated carbocyclylenyl, a 4-7 membered saturated or partially unsaturated heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
Ring X is a ring selected from phenylenyl, a 4-7 membered saturated or partially unsaturated carbocyclylenyl, a 4-7 membered saturated or partially unsaturated heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
each L^{B} and L² is independently a covalent bond or a bivalent, saturated or partially unsaturated, straight or branched C₁₋₅ hydrocarbon chain, wherein 0-3 methylene units of L^{B} or L^{2′} are independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, or —OC(O)NR—, —NRC(O)O—;
each R^{x}, R^{y}, and R^{z} is independently selected from hydrogen, R^{B}, halogen, CN, —NO₂, —OR, —SR, —NR₂, —S(O)₂R, —S(O)₂NR₂, —S(O)R, —C(O)R, —C(O)OR, —C(O)NR₂, —C(O)NROR, —OC(O)R, —OC(O)NR₂, —NRC(O)OR, —NRC(O)R, —NRC(O)N(R)₂, and —NRS(O)₂R;
each R^{B} is independently an optionally substituted group selected from C₁₋₆ aliphatic, phenyl, a 3-12 membered saturated or partially unsaturated monocyclic, bicyclic, bridged bicyclic or spirocyclic carbocyclyl or heterocyclyl with 1-3 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or;
two R groups on the same nitrogen are optionally taken together with their intervening atoms to form a 4-7 membered saturated, partially unsaturated, or heteroaryl ring having 0-3 heteroatoms, in addition to the nitrogen atom to which they are attached, independently selected from nitrogen, oxygen, and sulfur; and
x, y, and z are independently 0, 1, 2, 3, or 4; or
a compound of formula I-ii: 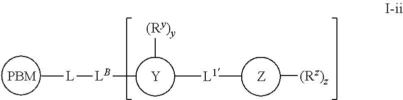
or a pharmaceutically acceptable salt thereof, wherein:
Ring Z is a ring selected from phenyl, a 4-7 membered saturated or partially unsaturated carbocyclic ring, a 4-7 membered saturated or partially unsaturated heterocyclic ring with 1-3 heteroatoms independently selected from nitrogen, oxygen, and sulfur, and a 5-6 membered heteroaromatic ring with 1-4 heteroatoms independently selected from nitrogen, oxygen and sulfur;
Ring Y is a ring selected from phenylenyl, a 4-7 membered saturated or partially unsaturated carbocyclylenyl, a 4-7 membered saturated or partially unsaturated heterocyclylenyl with 1-3 heteroatoms independently selected from nitrogen, oxygen, and sulfur, a 5-6 membered heteroarylenyl with 1-4 heteroatoms independently selected from nitrogen, oxygen and sulfur and an 8-10 membered bicyclic heterocyclylenyl having 1-5 heteroatoms independently selected from nitrogen, oxygen, or sulfur;
each L^{B} and L^{1′} is a covalent bond or a bivalent, saturated or partially unsaturated, straight or branched C₁₋₅ hydrocarbon chain, wherein 0-3 methylene units of L^{B} or L^{1′} are independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, or —OC(O)NR—, —NRC(O)O—;
each R^{y} and R^{z} is independently selected from hydrogen, R^{B}, halogen, CN, —NO₂, —OR, —SR, —NR₂, —S(O)₂R, —S(O)₂NR₂, —S(O)R, —C(O)R, —C(O)OR, —C(O)NR₂, —C(O)NROR, —OC(O)R, —OC(O)NR₂, —NRC(O)OR, —NRC(O)R, —NRC(O)N(R)₂, and —NRS(O)₂R;
each R^{B} is independently an optionally substituted group selected from C₁₋₆ aliphatic, phenyl, a 3-12 membered saturated or partially unsaturated monocyclic, bicyclic, bridged bicyclic or spirocyclic carbocyclyl or heterocyclyl with 1-3 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or;
two R groups on the same nitrogen are optionally taken together with their intervening atoms to form a 4-7 membered saturated, partially unsaturated, or heteroaryl ring having 0-3 heteroatoms, in addition to the nitrogen atom to which they are attached, independently selected from nitrogen, oxygen, and sulfur; and
y, and z are independently 0, 1, 2, 3, or 4; or
a compound of formula I-kk: 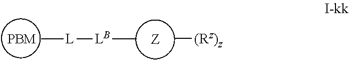
or a pharmaceutically acceptable salt thereof, wherein:
Ring Z is a ring selected from phenyl, a 4-7 membered saturated or partially unsaturated carbocyclic ring, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, and an 8-10 membered bicyclic heteroaromatic ring having 1-5 heteroatoms independently selected from nitrogen, oxygen, or sulfur;
each R^{z} is independently selected from hydrogen, R^{B}, halogen, CN, —NO₂, —OR, —SR, —NR₂, —S(O)₂R, —S(O)₂NR₂, —S(O)R, —C(O)R, —C(O)OR, —C(O)NR₂, —C(O)NROR, —OC(O)R, —OC(O)NR₂, —NRC(O)OR, —NRC(O)R, —NRC(O)N(R)₂, and —NRS(O)₂R;
L^{B} is a covalent bond or a bivalent, saturated or partially unsaturated, straight or branched C₁₋₅ hydrocarbon chain, wherein 0-3 methylene units of L^{B} are independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, or —OC(O)NR—, —NRC(O)O—;
each R^{B} is independently an optionally substituted group selected from C₁₋₆ aliphatic, phenyl, a 3-12 membered saturated or partially unsaturated monocyclic, bicyclic, bridged bicyclic or spirocyclic carbocyclyl or heterocyclyl with 1-3 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or;
two R groups on the same nitrogen are optionally taken together with their intervening atoms to form a 4-7 membered saturated, partially unsaturated, or heteroaryl ring having 0-3 heteroatoms, in addition to the nitrogen atom to which they are attached, independently selected from nitrogen, oxygen, and sulfur; and
z is 0, 1, 2, 3, or 4; or
a compound of formula I-II: 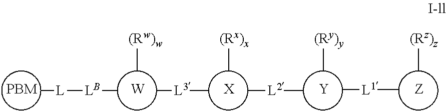
or a pharmaceutically acceptable salt thereof, wherein:
Ring Z is a ring selected from phenyl, a 4-7 membered saturated or partially unsaturated carbocyclic ring, a 4-7 membered saturated or partially unsaturated heterocyclic ring with 1-3 heteroatoms independently selected from nitrogen, oxygen, and sulfur, and a 5-6 membered heteroaromatic ring with 1-4 heteroatoms independently selected from nitrogen, oxygen and sulfur;
Ring Y is a ring selected from phenylenyl, a 4-7 membered saturated or partially unsaturated carbocyclylenyl, a 4-7 membered saturated or partially unsaturated heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
Ring X is a ring selected from phenylenyl, a 4-7 membered saturated or partially unsaturated carbocyclylenyl, a 4-7 membered saturated or partially unsaturated heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
Ring W is a ring selected from phenylenyl, a 4-7 membered saturated or partially unsaturated carbocyclylenyl, a 4-7 membered saturated or partially unsaturated heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
each L^{B}, L^{1′}, and L^{3′} is independently a covalent bond or a bivalent, saturated or partially unsaturated, straight or branched C₁₋₅ hydrocarbon chain, wherein 0-3 methylene units of L^{B}, L^{1′}, or L^{3′} are independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, or —OC(O)NR—, —NRC(O)O—;
each R^{w}, R^{x}, R^{y}, and R^{z} is independently selected from hydrogen, R^{B}, halogen, —CN, —NO₂, —OR, —SR, —NR₂, —S(O)₂R, —S(O)₂NR₂, —S(O)R, —C(O)R, —C(O)OR, —C(O)NR₂, —C(O)NROR, —OC(O)R, —OC(O)NR₂, —NRC(O)OR, —NRC(O)R, —NRC(O)N(R)₂, and —NRS(O)₂R;
each R^{B} is independently an optionally substituted group selected from C₁₋₆ aliphatic, phenyl, a 3-12 membered saturated or partially unsaturated monocyclic, bicyclic, bridged bicyclic or spirocyclic carbocyclyl or heterocyclyl with 1-3 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or;
two R groups on the same nitrogen are optionally taken together with their intervening atoms to form a 4-7 membered saturated, partially unsaturated, or heteroaryl ring having 0-3 heteroatoms, in addition to the nitrogen atom to which they are attached, independently selected from nitrogen, oxygen, and sulfur; and
w, x, y, and z are independently 0, 1, 2, 3, or 4; or
a compound of formula I-II′: 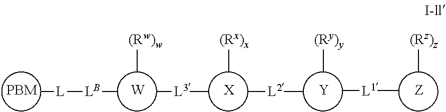
or a pharmaceutically acceptable salt thereof, wherein:
Ring Z is a ring selected from phenylenyl, a 3-7 membered saturated or partially unsaturated monocyclic, bicyclic, bridged bicyclic or spirocyclic carbocyclylenyl, a 3-7 membered saturated or partially unsaturated monocyclic, bicyclic, bridged bicyclic or spirocyclic heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
Ring Y is a ring selected from phenylenyl, a 4-7 membered saturated or partially unsaturated carbocyclylenyl, a 4-7 membered saturated or partially unsaturated heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
Ring X is a ring selected from phenylenyl, a 4-7 membered saturated or partially unsaturated carbocyclylenyl, a 4-7 membered saturated or partially unsaturated heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
Ring W is a ring selected from phenylenyl, a 4-7 membered saturated or partially unsaturated carbocyclylenyl, a 4-7 membered saturated or partially unsaturated heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
each L^{B}, L^{1′}, and L^{3′} is independently a covalent bond or an optionally substituted bivalent, saturated or partially unsaturated, straight or branched C₁₋₅ hydrocarbon chain, wherein 0-3 methylene units of L^{B}, L^{1′}, or L^{3′} are independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, or —OC(O)NR—, —NRC(O)O—;
each R^{w}, R^{x}, R^{y}, and R^{z} is independently selected from hydrogen, R^{B}, halogen, —CN, —NO₂, —OR, —SR, —NR₂, —S(O)₂R, —S(O)₂NR₂, —S(O)R, —C(O)R, —C(O)OR, —C(O)NR₂, —C(O)NROR, —OC(O)R, —OC(O)NR₂, —NRC(O)OR, —NRC(O)R, —NRC(O)N(R)₂, and —NRS(O)₂R;
each R^{B} is independently an optionally substituted group selected from C₁₋₆ aliphatic, phenyl, a 3-12 membered saturated or partially unsaturated monocyclic, bicyclic, bridged bicyclic or spirocyclic carbocyclyl or heterocyclyl with 1-3 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or;
two R groups on the same nitrogen are optionally taken together with their intervening atoms to form a 4-7 membered saturated, partially unsaturated, or heteroaryl ring having 0-3 heteroatoms, in addition to the nitrogen atom to which they are attached, independently selected from nitrogen, oxygen, and sulfur;
p is independently 0 or 1; and
w, x, y, and z are independently 0, 1, 2, 3, or 4.

25-34. (canceled)

35. The compound of claim 24, wherein ring W is cyclohexyl.

36. The compound of claim 24, wherein ring X is a phenylenyl.

37. The compound of claim 24, wherein ring Y is selected from the group consisting of phenylenyl,

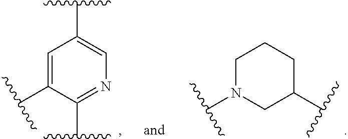 [SMILES: *c1cnc(*)c(*)c1 ; *C1CCCN(*)C1]

38-41. (canceled)

42. The compound of 24, wherein ring Z is a phenyl or cyclopropyl.

43-45. (canceled)

46. The compound of claim 24, wherein said compound is a compound of any of the following formulae:

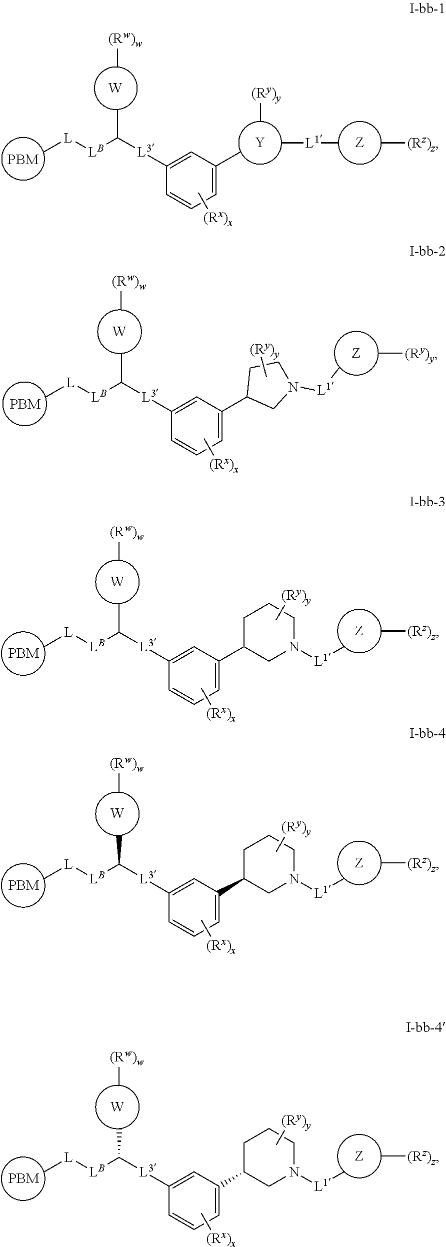

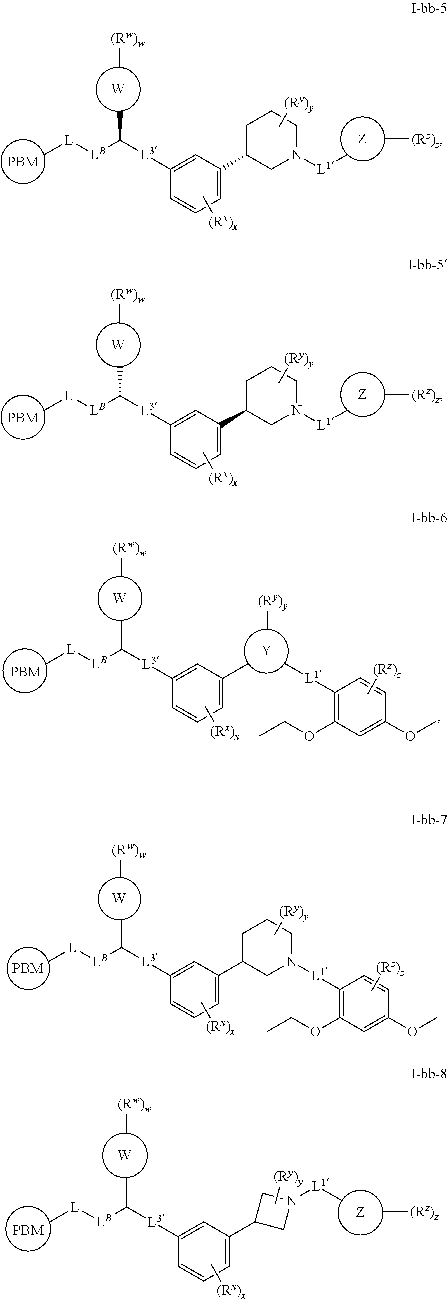

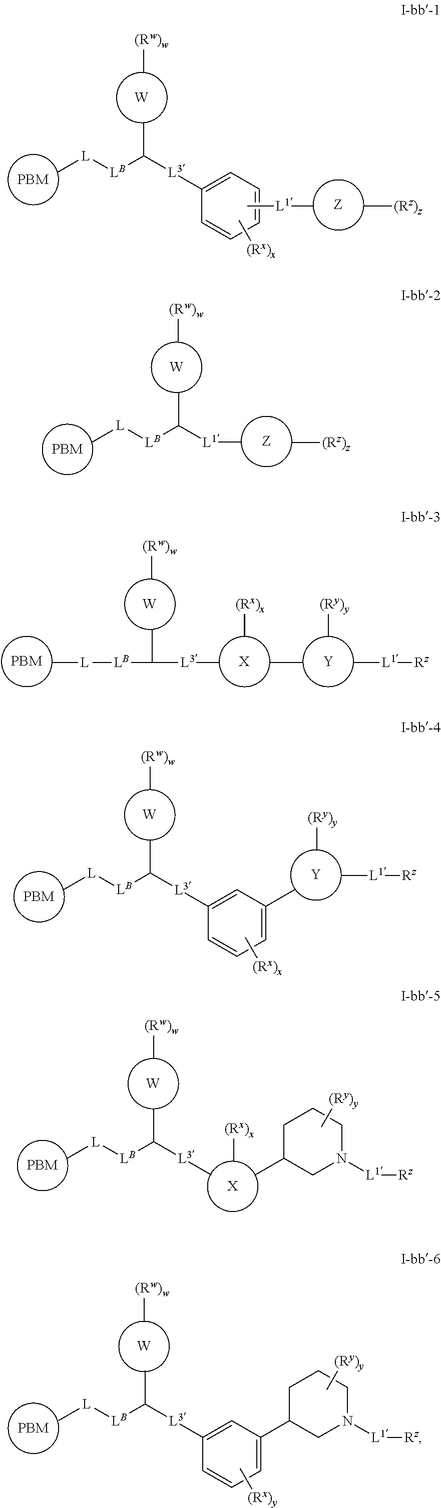

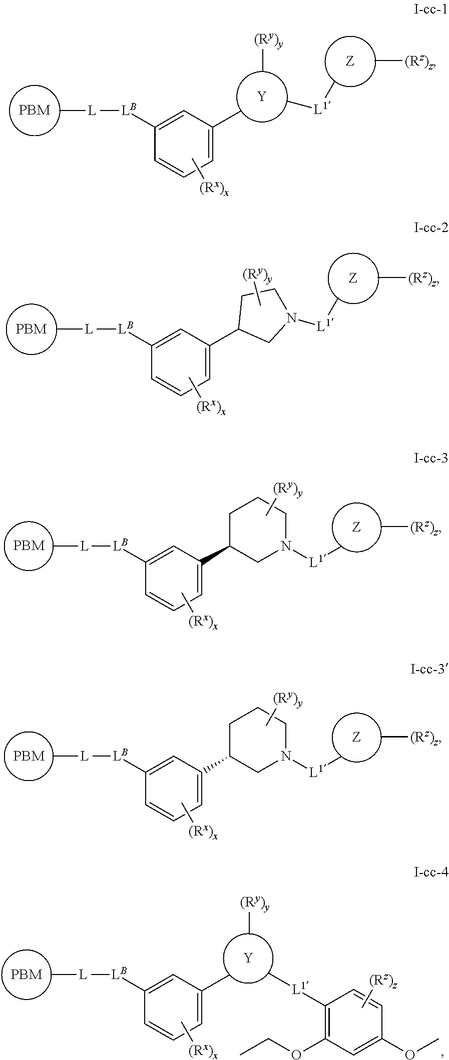

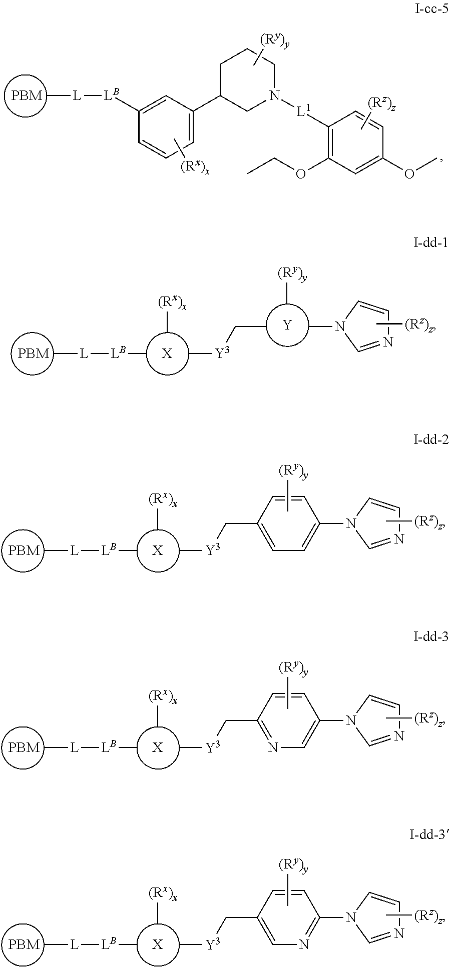

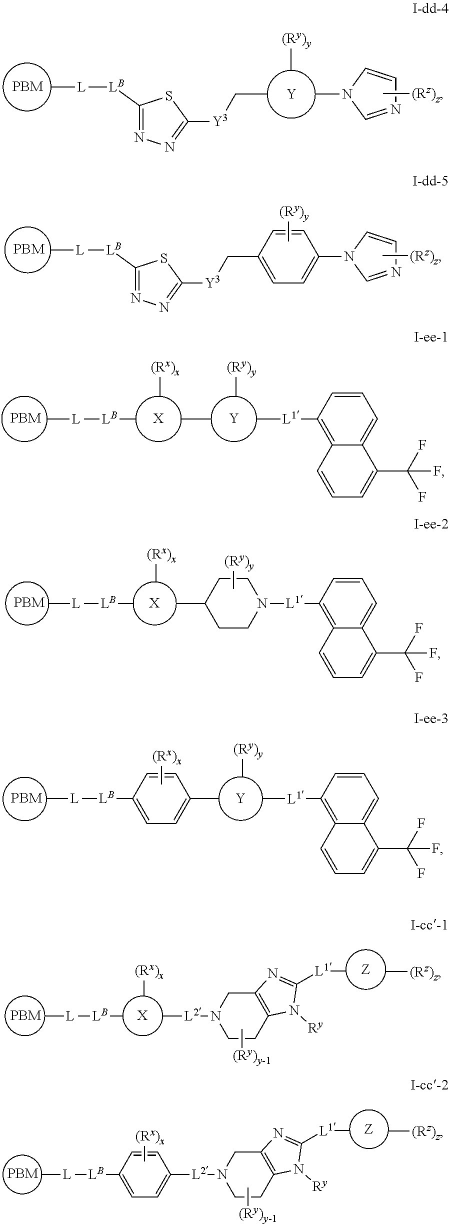

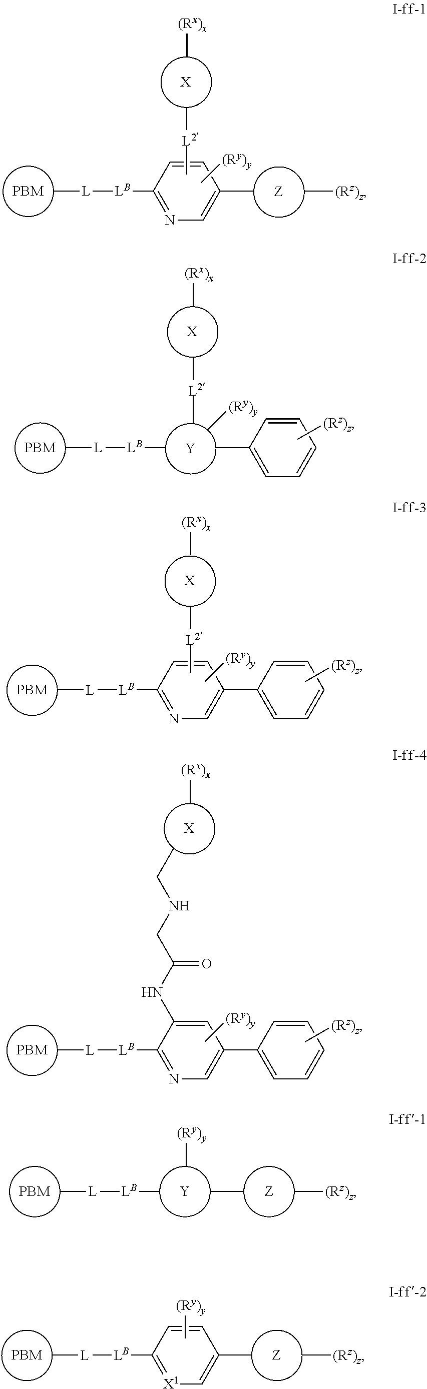

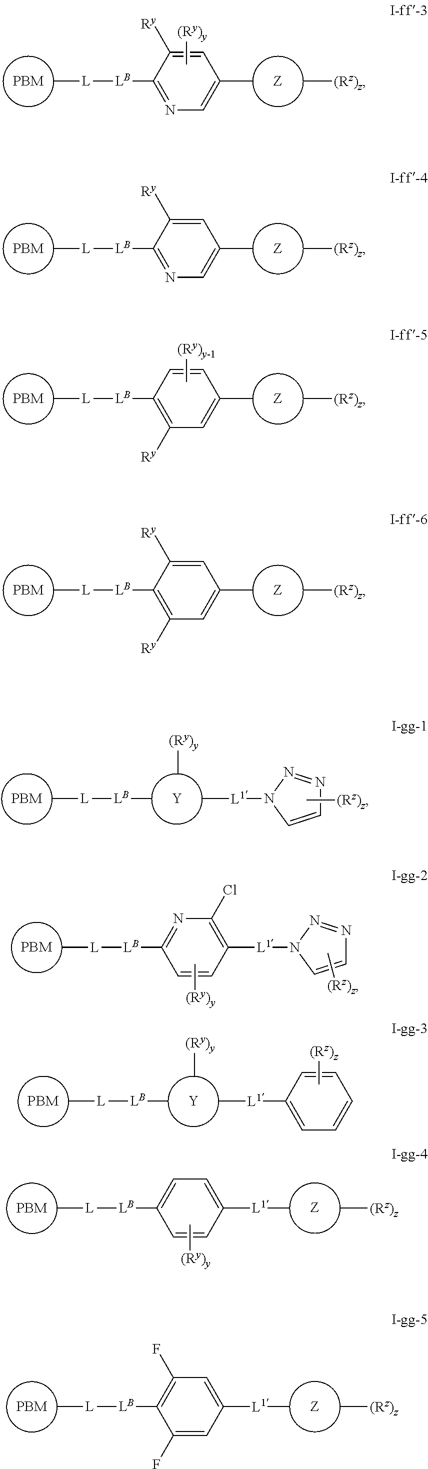

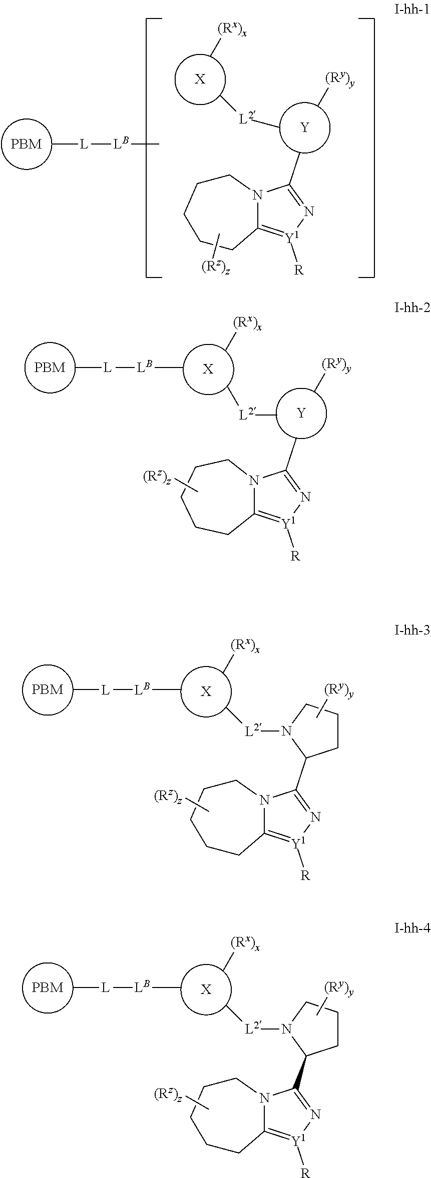

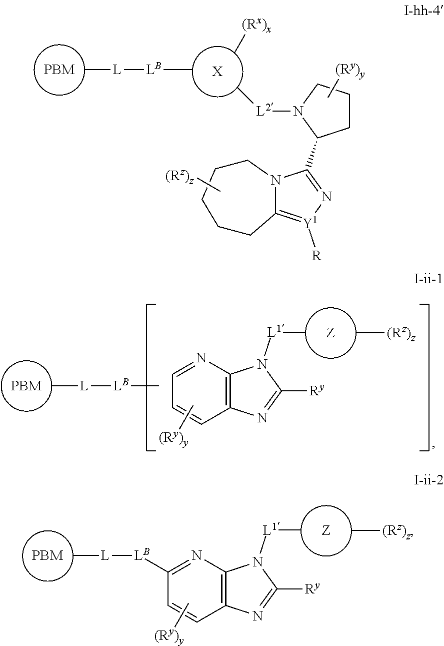

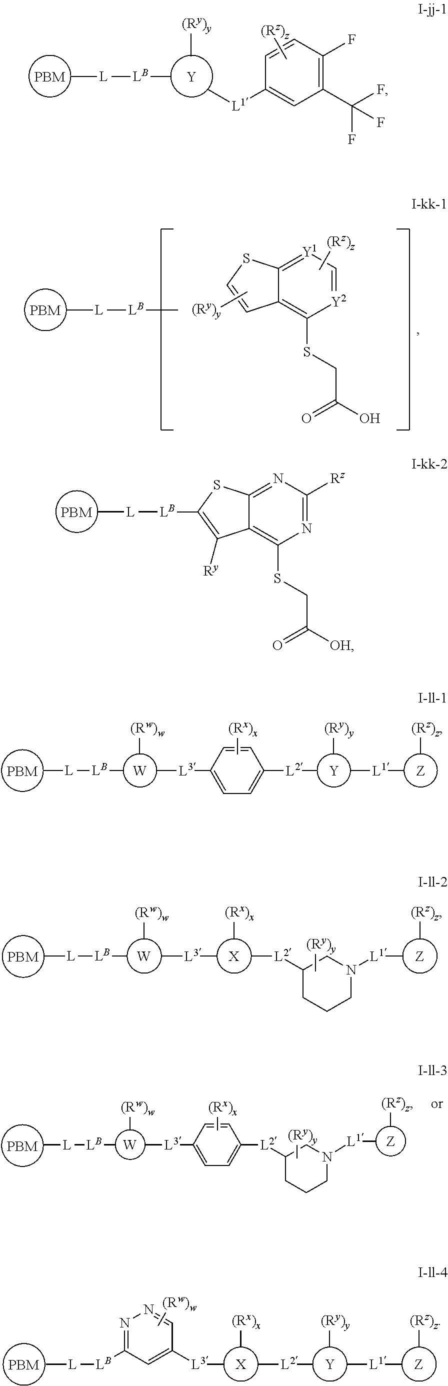

47-56. (canceled)

57. The compound of claim 3, wherein L is a covalent bond or a bivalent, saturated or partially unsaturated, straight or branched C₁₋₂₀ hydrocarbon chain, wherein 0-6 methylene units of L are independently replaced by -Cy-, —O—, —NR—, —S—, —OC(O)—, —C(O)O—, —C(O)—, —S(O)—, —S(O)₂—, —N(R)S(O)₂—, —S(O)₂N(R)—, —N(R) C(O)—, —C(O)N(R)—, —OC(O)N(R)—, —N(R)C(O)O—.

58. The compound of claim 57, wherein L is selected from —C(O)—CH₂-Cy-CH₂-Cy-, —C(O)—CH₂-Cy-O-Cy-, —C(O)—CH₂-Cy, -Cy-CH₂-Cy-, and -Cy-O-Cy-.

59-62. (canceled)

63. The compound of claim 24, wherein DBM is selected from those depicted in Table A.

64. (canceled)

65. The compound of claim 1, wherein said compound is selected from any one of the compounds depicted in Table 1, or a pharmaceutically acceptable salt thereof.

66. A pharmaceutical composition comprising a compound of claim 1, and a pharmaceutically acceptable carrier, adjuvant, or vehicle.

67. The pharmaceutical composition according to claim 66, further comprising an additional therapeutic agent.

68. A method of inhibiting AKT in a patient or biological sample comprising administering to said patient, or contacting said biological sample, with a compound according to claim 1, or a pharmaceutical composition thereof.

69. A method of treating a AKT-mediated disorder, disease, or condition in a patient comprising administering to said patient a compound according to of claim 1, or a pharmaceutical composition thereof.

70. The method of claim 69, wherein AKT-mediated disorder, disease, or condition is cancer.

71. The method of claim 70, wherein the cancer the cancer is characterized by amplification or overexpression of AKT.
